(19) United States
(12) Patent Application Publication
(10) Pub. No.: US 2024/0174683 A1
(43) Pub. Date: May 30, 2024

(54) MAP4K1 INHIBITORS

(71) Applicants: Bayer Aktiengesellschaft, Leverkusen (DE); Deutsches Krebsforschungszentrum, Heidelberg (DE)

(72) Inventors: Ulrich LÜCKING, Berlin (DE); Jeffrey Stuart MOWAT, Antwerp (BE); Ludwig ZORN, Berlin (DE); Lars WORTMANN, Biberbach an der Riß (DE); Steffen MÜLLER, Mülheim an der Ruhr (DE); Gabriele LEDER, Berlin (DE); Sandra BERNDT, Hohen Neuendorf (DE); Judith GÜNTHER, Berlin (DE); Lara KUHNKE, Berlin (DE); Antje Margret WENGNER, Berlin (DE); Rafael CARRETERO, Heidelberg (DE); Gerd WOHLFAHRT, Berlin (DE); Anders FRIBERG, Berlin (DE); Ulf BÖMER, Glienicke/Nordbahn (DE); Roland NEUHAUS, Berlin (DE); Maren OSMERS, Dallgow-Döberitz (DE); Hideki Miyatake ONDOZABAL, Berlin (DE); Martina SCHÄFER, Berlin (DE); Louise EAGLING, Berlin (DE); Julien LEFRANC, Darmstadt (DE); Katrin NOWAK-REPPEL, Berlin (DE); Rienk OFFRINGA, Heidelberg (DE); Peng CHEN, Xian City (CN); Xuewei WANG, Xian City (CN); Yuanyuan YAN, Xian City (CN); Kai THEDE, Berlin (DE); Nikolay SITNIKOV, Schulzendorf (DE); Berndt BUCHMANN, Hohen Neuendorf (DE); Dirk KOSEMUND, Berlin (DE); Detlef STOECKIGT, Potsdam (DE)

(73) Assignees: Bayer Aktiengesellschaft, Leverkusen (DE); Deutsches Krebsforschungszentrum, Heidelberg (DE)

(21) Appl. No.: 18/275,978

(22) PCT Filed: Feb. 4, 2022
(86) PCT No.: PCT/EP2022/052794

§ 371 Date: Aug. 4, 2023

(30) Foreign Application Priority Data
Feb. 5, 2021 (WO) PCT/CN2021/075566
Apr. 13, 2021 (WO) PCT/CN2021/086946

(51) Int. Cl. C07D 487/20; A61K 45/06; C07B 59/00; C07D 498/20

(52) U.S. Cl. CPC C07D 487/20; A61K 45/06; C07B 59/002; C07D 498/20; C07B 2200/13

(57) ABSTRACT

The present invention relates to Map4K1 inhibitors of formula (I) (I), wherein A, E, G, Q, R¹, R² and R⁴ have the same meaning as defined in the description, to pharmaceutical compositions and combinations comprising the compounds according to the invention, and to the prophylactic and therapeutic use of the inventive compounds, respectively to the use of said compounds for manufacturing pharmaceutical compositions for the treatment or prophylaxis of diseases, in particular for neoplastic disorders, respectively cancer or conditions with dysregulated immune responses or other disorders associated with aberrant MAP4K1 signaling, as a sole agent or in combination with other active ingredients. The present invention further relates to the use, respectively to the use of said compounds for manufacturing pharmaceutical compositions for the treatment or prophylaxis of protein inhibitors in benign hyperplasias, atherosclerotic disorders, sepsis, autoimmune disorders, vascular disorders, viral infections, in neurodegenerative disorders, in inflammatory disorders, in atherosclerotic disorders and in male fertility control.

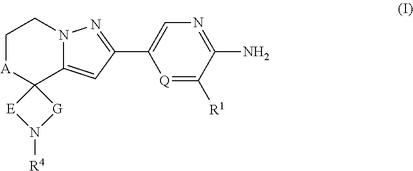

The present invention relates to MAP4K1 inhibitors, to pharmaceutical compositions and combinations comprising the compounds according to the invention, and to the prophylactic and therapeutic use of the inventive compounds, respectively to the use of said compounds for manufacturing pharmaceutical compositions for the treatment or prophylaxis of diseases, in particular for neoplastic disorders, respectively cancer or conditions with dysregulated immune responses or other disorders associated with aberrant MAP4K1 signaling, as a sole agent or in combination with other active ingredients.

The present invention further relates to the use, respectively to the use of said compounds for manufacturing pharmaceutical compositions for the treatment or prophylaxis of protein inhibitors in benign hyperplasias, atherosclerotic disorders, sepsis, autoimmune disorders, vascular disorders, viral infections, in neurodegenerative disorders, in inflammatory disorders, in atherosclerotic disorders and in male fertility control.

### BACKGROUND

Although cancer cell commonly can be recognized by the adaptive immune system, the response generated is evidently not capable of eliminating the tumor. A major reason for this is the presence of immunosuppressive mechanisms in the tumor microenvironment. In this respect, inhibitors of T-cell immune checkpoint such as CTLA-4, PD-1 or PD-L1 were recently shown to result in a remarkable clinical efficacy in subsets of cancer patients. Besides cell surface receptors that act as negative immune regulators, several mediators of intracellular signaling have been identified that also represent potential immunoevasive mechanisms utilized by the tumor.

One of these is MAP4K1, also known as hematopoietic progenitor kinase 1 (HPK1). MAP4K1 (GeneID11184) is a serine/threonine kinase and member of the Germinal Center Kinase family. In the adult organism MAP4K1 expression is restricted to hematopoietic cell types. The MAP4K1 protein consist of a N-terminal kinase domain, followed by a proline-rich domain that can interact with adaptor molecules through SH2 and SH3 domains, and a C-terminal citron homology domain of which the exact function remains to be identified. Through its proline-rich domain, MAP4K1 is capable of binding to a diversity of adaptors in hematopoietic cells, including those involved in T-cell receptor (TCR), B-cell receptor (BCR) and cytokine signaling (Hu et al., Genes Dev. 1996 Sep. 15; 10(18):2251-64, 2; Ling et al., J Biol Chem. 2001 Jun. 1; 276(22), Sauer et al., J Biol Chem. 2001 Nov. 30; 276(48):45207-16., Tsuji et al., J Exp Med. 2001 Aug. 20; 194(4):529-39, Boomer et al., J Cell Biochem. 2005 May 1; 95(1):34-44).

The function of MAP4K1 has been studied in greatest detail in the context of TCR signaling. Upon TCR stimulation, MAP4K1 is phosphorylated on tyrosine 381 (Y-381; Y-379 in mouse) (Di Bartolo et al., J Exp Med. 2007 Mar. 19; 204(3):681-91). Consequently, MAP4K1 is recruited to the TCR-signaling complex where it induces dissociation of this complex through its serine/threonine kinase function. In particular MAP4K1 phosphorylates the SLP-76 adaptor protein at Serine-376, resulting in downregulation of AP-1 and Erk2 pathways. As, such, MAPK1 acts as a negative feedback on TCR-signaling (Liou et al., Immunity. 2000 April; 12(4):399-408; Lasserre et al., J Cell Biol. 2011 Nov. 28; 195(5):839-53). Alternatively, MAP4K1 can be triggered to suppress T cell function by prostaglandin E2 (PGE2), and possibly also by transforming growth factor beta (TGF-beta), factors that are commonly found in the tumor microenvironment. Notably, MAP4K1 activation by these mediators involves protein kinase A (PKA)-dependent phosphorylation of Serine 171 (5-171; also in mouse) (Alzabin et al., Cancer Immunol Immunother. 2010 March; 59(3):419-29; Sawasdikosol et al., J Biol Chem. 2007 Nov. 30; 282(48):34693-9).

Further important insights into the function of MAP4K1 in the regulation of T cell immunity stem from in vivo and in vitro experiments respectively with MAP4K1 deficient mice produced by two laboratories and with immune cells isolated from these mice (Shui et al., Nat Immunol. 2007 January; 8(1):84-91; Alzabin et al., Cancer Immunol Immunother. 2010 March; 59(3):419-29). MAP4K1-deficient mice show an apparent normal phenotype, are fertile and exhibit normal lymphocyte development. These animals are prone to develop T-cell dependent autoimmune reactivity as indicated by development of a more severe disease score in the EAE (experimental autoimmune encephalomyelitis) model of multiple sclerosis (Shui et al., Nat Immunol. 2007 January; 8(1):84-91). In case of the second strain, a dysregulation of immune function was observed when, at the age of approximately 6 months, MAP4K1-deficient mice develop a spontaneous autoimmune phenotype (Alzabin et al., Cancer Immunol Immunother. 2010 March; 59(3):419-29). In vitro studies showed that MAP4K1−/− T-cells display hyper-responsiveness upon TCR-stimulation. These cells proliferate and secrete pro-inflammatory cytokines like IL-2 or IFNg to a significantly greater extent than their wild-type counterparts (Shui et al., Nat Immunol. 2007 January; 8(1):84-91). Furthermore, MAP4K1−/− T-cells are resistant to PGE2-mediated suppression of T cell proliferation, suppression of IL-2 production and induction of apoptosis (Alzabin et al., Cancer Immunol Immunother. 2010 March; 59(3):419-29). In the context of tumor immunology, in vivo experiments revealed that MAP4K1−/− mice are much more resistant to tumorigenesis by PGE2-producing Lewis lung carcinoma than wild type mice, which correlated with increased T-lymphocyte infiltration in the tumor areas. The crucial role of T-cells in tumor rejection was supported by experiments in which MAP4K1−/− T-cells adoptively transferred into T-cell-deficient mice were able to eradicate tumors more efficiently than wild-type T-cells (Alzabin et al., Cancer Immunol Immunother. 2010 March; 59(3):419-29). The important role of the kinase enzymatic activity was demonstrated by studies were only wild type MAP4K1, but not the MAP4K1 kinase-dead mutant, could mediate serine-phosphorylation of the TCR-signaling complex component SLP-76 and subsequent binding of SLP-76 to the negative regulator of TCR-signaling 14-3-3-t (Shui et al., Nat Immunol. 2007 January; 8(1):84-91). MAP4K1 also regulates the stimulation and activation of dendritic cells. MAP4K1 deficient Bone marrow derived cells (BMDC) express after maturation and stimulation higher level of costimulatory molecules and produce more proinflammatory cytokines. Also elimination of tumors was observed to be more efficient by MAP4K1−/− BMDC compared to their wildtype counterparts (Alzabin et al., J Immunol. 2009 May 15; 182(10):6187-94).

### PRIOR ART

In WO2019164846A1 HPK1 inhibitors and methods for their use in various forms of cancer are described. These compounds differ from the instant compounds in their chemical structure.

In US20190256500A1 HPK1 inhibitors and methods for their use in treating, preventing or ameliorating diseases or disorders associated with HPK1 such as cancer are described. These compounds differ from the instant compounds in their chemical structure.

In US20190256520A1 HPK1 inhibitors and methods for their use in treating, preventing or ameliorating diseases or disorders associated with HPK1 such as cancer are described. These compounds differ from the instant compounds in their chemical structure.

In CN109721620A HPK1 inhibitors and their uses are described. These compounds differ from the instant compounds in their chemical structure.

In WO2019090198A1, compounds used to modulate or inhibit the activity of HPK1 and methods for their use in treatment of viral infections and proliferative disorders, such as cancer are described. These compounds differ from the instant compounds in their chemical structure.

In WO 2018/215668, MAP4K1 (HPK1) inhibitors and methods for their use in diseases including hyperproliferative diseases, diseases of immune system dysfunction, inflammatory disorders, neurological diseases, and cardiovascular diseases are described. These compounds differ from the instant compounds in their chemical structure.

In WO 2018/049214, HPK1 modulators and methods for their use in cancer treatment are described. These compounds differ from the instant compounds in their chemical structure.

In WO 2018/049200, HPK1 modulators and methods for their use in cancer treatment are described. These compounds differ from the instant compounds in their chemical structure.

In WO 2018/049152, HPK1 modulators and methods for their use in cancer treatment are described. These compounds differ from the instant compounds in their chemical structure.

In WO 2018/049119, HPK1 modulators and methods for their use in cancer treatment are described. These compounds differ from the instant compounds in their chemical structure.

In WO 2018/102366, HPK1 inhibitors and methods for their use in the treatment of cancer are described. These compounds differ from the instant compounds in their chemical structure.

In WO 2018/183956, HPK1 inhibitors and use of such compounds in treating HPK1-dependent disorders and enhancing immune response are described. These compounds differ from the instant compounds in their chemical structure.

In WO 2018/183964, HPK1 inhibitors and use of such compounds in treating HPK1-dependent disorders and enhancing immune response are described. These compounds differ from the instant compounds in their chemical structure.

In WO 2018/167147, HPK1 inhibitors and use of such compounds in treating HPK1-dependent disorders and enhancing immune response are described. These compounds differ from the instant compounds in their chemical structure.

In WO2016/205942 HPK1, respectively inhibitors and methods of their use in cancer treatment are described. Specifically, the application concerns thieno-pyridinones that can be used in anti-cancer therapy. These compounds differ from the instant compounds in their chemical structure.

In WO 2016/195776 inhibitors and methods for leukemia, cancer and diabetes treatment dependent on inhibition the interaction of menin with of MLL1, MLL2 and MLL-fusion oncoproteins are described. These compounds differ from the instant compounds in their chemical structure.

In WO 2006/014325 C-MET modulators and their use in cancer treatment are described. These compounds differ from the instant compounds in their chemical structure.

In WO 2005/058891 Rho kinase inhibitors and their use in cardiovascular and cancer treatment are described. These compounds differ from the instant compounds in their chemical structure.

In WO 2015/089479 several inhibitors are described that show inhibition of several kinases (e.g., BTK, HCK, TAK1 and HPK1). These compounds differ from the instant compounds in their chemical structure.

In WO2016/004272 BTK inhibitors and methods of their use in cancer treatment are described. No specific example is disclosed which falls in the group of compounds as defined according to the present invention.

In WO 2011/090738 Type II RAF kinase inhibitors and their use in various diseases are described. No specific example is disclosed which falls in the group of compounds as defined according to the present invention.

In CN102086211 and WO2006116713 protein kinase inhibitors and their use in prophylaxis and treatment of diseases including cancer are described. No specific example is disclosed which falls in the group of compounds as defined according to the present invention.

In WO 2010/045095 protein tyrosin kinase modulators and their use in the treatment of hyperproliferative disorders are described. No specific example is disclosed which falls in the group of compounds as defined according to the present invention.

In WO 2008/089307 compounds and methods of their use in the treatment of pain, inflammation and cancer are described. No specific example is disclosed which falls in the group of compounds as defined according to the present invention.

In WO 2006/114180 kinase inhibitors for treating diseases, particularly tumors are described. No specific example is disclosed which falls in the group of compounds as defined according to the present invention.

In WO 2006/014325 c-Met modulators and their methods of use to treat kinase-dependent diseases and conditions are described. No specific example is disclosed which falls in the group of compounds as defined according to the present invention.

In US 2003/0055049 compounds for treating disorders with abnormal cell growth in mammals are described. No specific example is disclosed which falls in the group of compounds as defined according to the present invention.

In WO 2001/23389 antagonists of NPY receptors compositions and methods of the treatment of physiological disorders associated with an excess of neuropeptide Y are described. No specific example is disclosed which falls in the group of compounds as defined according to the present invention.

In WO 2019/149738 protein kinase MKK4 inhibitors for promoting liver regeneration or reducing or preventing hepatocyte death are described.

It would therefore be desirable to provide novel MAP4K1 inhibitors having prophylactic and therapeutic properties.

Accordingly, it is an object of the present invention to provide compounds and pharmaceutical compositions comprising these compounds used for prophylactic and therapeutic applications for hyperproliferative disorders, in particular for cancer, respectively tumour disorders, and conditions with dysregulated immune responses, as a sole agent or in combination with other active ingredients.

A further object of the present invention is to provide compounds and pharmaceutical compositions comprising these compounds for manufacturing pharmaceutical compositions for the treatment or prophylaxis of benign hyperplasias, atherosclerotic disorders, sepsis, autoimmune disorders, vascular disorders, viral infections, in neurodegenerative disorders, in inflammatory disorders, in atherosclerotic disorders and in male fertility control.

Surprisingly, the compounds according to the invention inhibit the MAP4K1 protein and thereby enhance tumor immunogenicity leading to inhibition of cancer cells growth by the immune response. Accordingly, they provide novel structures for the therapy of human and animal disorders, in particular of cancers.

The present invention relates to compound of formula (I) (if referred to embodiment 1, this group of compounds is meant)

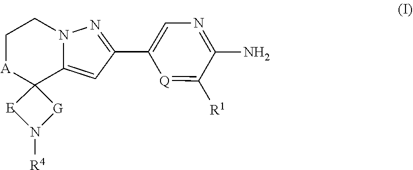

wherein
A is selected from a single bond, —CH₂— or —O—;
E is —(CH₂)ₙ— with n=1 or 2;
G is —(CH₂)— and with the condition, that the cyclic substructure comprising E-N-G is a 4 to 5 membered heterocycloalkyl;
Q is selected from N or C—R²;
R¹ is selected from
   —NO₂; —CN;
   C₁₋₄-alkyl optionally substituted by —OH or 1 to 3-F;
   cyclopropyl;
   4 to 6 membered heterocycloalkyl or heterocycloalkenyl, both with 1-2 nitrogen atoms, but always linked via a carbon atom to the phenyl group of compounds of formula (I) and both optionally substituted by C₁₋₄-alkyl;
   phenyl optionally substituted by halogen, —CN, C₁₋₄-alkyl, C₁₋₄-haloalkyl, C₃₋₆-cycloalkyl, O—C₁₋₄-alkyl, O—C₃₋₆-cycloalkyl, C₁₋₃-alkinyl, —C(O)—R_{d}, SR_{d}, SOR_{d}, SO₂R_{d}, SONR_{d}, SON(H)R_{d}, —SO₂—NH₂, —SO₂—N(H)—R_{d} or —SO₂—N(R_{d})₂, wherein R_{d} is selected from methyl, ethyl, cyclopropyl, isopropyl and tert·butyl;
   5 to 6 membered heteroaryl with 1, 2 or 3 heteroatoms selected from N, O or S and optionally substituted by 1 or 2 of the following substituents: halogen, —CN, C₁₋₄-alkyl, C₁₋₄-haloalkyl, C₃₋₆-cycloalkyl, O—C₁₋₄-alkyl, O—C₃₋₆-cycloalkyl, C₁₋₃-alkinyl, SR_{d}, SOR_{d}, SO₂R_{d}, SONR_{d} or SON(H)R_{d} wherein R_{d} is selected from methyl, ethyl, cyclopropyl, isopropyl and tert·butyl;
   —O—CH₃; —O—CH(CH₃)₂; —O—CHF₂; —O—CF₃;

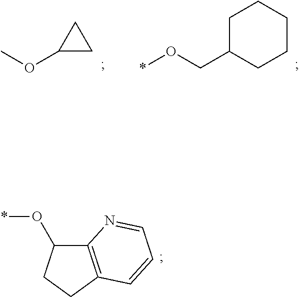

—O—CHRₐ-phenyl optionally substituted by one, two or three of the following substituents: halogen, —CN, C₁₋₄-alkyl optionally substituted by one or more —F, O—C₁₋₄-alkyl, C₁₋₆-alkinyl-OH or —SO₂—CH₃ and with Rₐ selected from —H, —F or —CH₃, —CF₃, —CH₂—CH₃, —CH(CH₃)₂ or -cyclopropyl;
—O—CHRₐ-6 membered heteroaryl optionally substituted one, two or three of the following substituents: halogen, —CN, C₁₋₄-alkyl optionally substituted by one or more —F or —SO₂—CH₃ and with Rₐ selected from —H, —F or —CH₃, —CF₃, —CH₂—CH₃, —CH(CH₃)₂ or -cyclopropyl;
—COORₐ with Rₐ selected from —H or —CH₃;
C(═O)-methyl, optionally substituted by 1 to 3-F;
—C(═O)—NR_{b}R_{c} wherein R_{b} is selected from —H, —CH₃ or —CH₂—CH₃ and wherein R_{c} is selected from —H, —CH₃, —CH₂—CH₃, cyclohexyl, phenyl or 5 to 6 membered heteroaryl, all optionally substituted by one or two halogen, —CN, —SO₂—CH₃ or C₁₋₄-alkyl, optionally substituted one or more times with halogen;
—S—CR_{f}R_{g}Rₕ, wherein R_{f} is selected from —H or —F, wherein R_{g} is selected from —H, —F or —CH₃ and wherein Rₕ is selected from H, F, —CH₃ or C₅₋₆-cycloalkyl or phenyl;

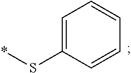 [SMILES: *Sc1ccccc1]

R² is selected from —H;
R⁴ is selected from
   C₁₋₆-alkyl, C₃₋₆-cycloalkyl or C₁₋₃-alkyl-C₃₋₆-cycloalkyl, all optionally substituted one or independently more times by
      —F, —Cl, —Br, —CN;
      —OH, —ORᵢ wherein Rᵢ is selected from C₁₋₄-alkyl or C₃₋₆-Cycloalkyl, both optionally substituted one or more times by halogen,
      —CF₃, —CHF₂, —CH₂F, or —CH₂—CF₃;
      —NRⱼRₖ wherein Rⱼ and Rₖ are independently selected from —H and C₁-C₄-alkyl,
      —CH₂—O—CH₃ or
      C₃₋₄ cycloalkyl or 4-6 membered heterocycloalkyl, both optionally substituted by (═O), or optionally substituted one or more times by F or CH₃;
      C₆₋₈-spiro-alkyl and C₅₋₇-bicyclo-alkyl
      phenyl or 5 to 6 membered heteroaryl, both optionally substituted one or more times by
         halogen, —CN,
         —CF₃, —CHF₂, —CH₂F,
         C₁₋₄-alkyl optionally substituted with one or more halogen;
         C₃₋₆-cycloalkyl;
         O—C₁₋₄-alkyl,
         O—C₃₋₆-cycloalky,
         C₁₋₃-alkinyl,
         —NRⱼRₖ wherein Rⱼ and Rₖ are independently selected from —H and C₁-C₄-alkyl
         SR_{d}, SOR_{d}, SO₂R_{d}, SONR_{d} or SON(H)R_{d} wherein R_{d} is selected from methyl, ethyl, cyclopropyl, isopropyl and tert·butyl; or
      phenyl is optionally substituted one or more times by —C(O)NRⱼRₖ wherein Rⱼ and Rₖ are independently selected from —H and C₁-C₄-alkyl or
      5 to 6 membered heteroaryl is optionally substituted one or more times by —OH;
   C(═O)—Rₗ in which Rₗ is selected from
      C₁₋₄-alkyl, C₃₋₆-cycloalkyl or C₃₋₆-heterocycloalkyl, both optionally substituted one or more times by
         —F, —Cl, —Br, —CN;
         —OH, —ORᵢ with Rᵢ selected from C₁₋₄-alkyl or C₃₋₆Cycloalkyl), —CF₃, —CHF₂, —CH₂F;
         —NRⱼRₖ wherein Rⱼ and Rₖ are independently selected from —H and C₁-C₄-alkyl
         phenyl or 5 to 6 membered heteroaryl, both optionally substituted one or more times by
         halogen, —CN,
         —CF₃, —CHF₂, —CH₂F,
         C₁₋₄-alkyl optionally substituted with one or more halogen;
         C₃₋₆-cycloalkyl;
         O—C₁₋₄-alkyl,
         O—C₃₋₆-cycloalky,
         C₁₋₃-alkinyl,
         —NRⱼRₖ wherein Rⱼ and Rₖ are independently selected from —H and C₁-C₄-alkyl
         SR_{d}, SOR_{d}, SO₂R_{d}, SONR_{d} or SON(H)R_{d} wherein R_{d} is selected from methyl, ethyl, cyclopropyl, isopropyl and tert·butyl;
      phenyl or 5 to 6 membered heteroaryl, both optionally substituted one or more times by
         halogen, —CN,
         —CF₃, —CHF₂, —CH₂F,
         C₁₋₄-alkyl optionally substituted with one or more halogen;
         C₃₋₆-cycloalkyl;
         O—C₁₋₄-alkyl,
         O—C₃₋₆-cycloalky,
         C₁₋₃-alkinyl,
         —NRⱼRₖ wherein Rⱼ and Rₖ are independently selected from —H and C₁-C₄-alkyl
         SR_{d}, SOR_{d}, SO₂R_{d}, SONR_{d} or SON(H)R_{d} wherein R_{d} is selected from methyl, ethyl, cyclopropyl, isopropyl and tert·butyl;
   C(═O)—NRₒRₚ wherein Rₒ and Rₚ are independently selected from
      H
      C₁₋₄-alkyl, C₃₋₆-cycloalkyl or C₁₋₃-alkyl-C₃₋₆-cycloalkyl, both optionally substituted one or more times by
         —F, —Cl, —Br, —CN;
         —OH, —ORᵢ with Rᵢ selected from C₁₋₄-alkyl or C₃₋₆-cycloalkyl, —CF₃, —CHF₂, —CH₂F;
         —NRⱼRₖ wherein Rⱼ and Rₖ are independently selected from —H and C₁-C₄-alkyl
         phenyl or 5 to 6 membered heteroaryl, both optionally substituted one or more times by
         halogen, —CN,
         —CF₃, —CHF₂, —CH₂F,
         C₁₋₄-alkyl optionally substituted with one or more halogen;
         C₃₋₆-cycloalkyl;
         O—C₁₋₄-alkyl,
         O—C₃₋₆-cycloalky,
         C₁₋₃-alkinyl,
         —NRⱼRₖ wherein Rⱼ and Rₖ are independently selected from —H and C₁-C₄-alkyl
         SR_{d}, SOR_{d}, SO₂R_{d}, SONR_{d} or SON(H)R_{d} wherein R_{d} is selected from methyl, ethyl, cyclopropyl, isopropyl and tert·butyl;
   phenyl or 5 to 6 membered heteroaryl, both optionally substituted one or more times by
      halogen, —CN,
      —CF₃, —CHF₂, —CH₂F,
      C₁₋₄-alkyl optionally substituted with one or more halogen;
      C₃₋₆-cycloalkyl;
      O—C₁₋₄-alkyl,
      O—C₃₋₆-cycloalky,
      C₁₋₃-alkinyl,
      NRⱼRₖ wherein Rⱼ and Rₖ are independently selected from —H and C₁-C₄-alkyl
      SR_{d}, SOR_{d}, SO₂R_{d}, SONR_{d} or SON(H)R_{d} wherein R_{d} is selected from methyl, ethyl, cyclopropyl, isopropyl and tert·butyl;

or a stereoisomer, a tautomer, an N-oxide, a hydrate, a solvate, or a salt thereof, or a mixture of same.

The compounds of formula (I) are particularly suitable for a large number of prophylactic and therapeutic applications, in particular for hyperproliferative disorders, for tumour disorders and as proteine inhibitors and further for viral infections, for neurodegenerative disorders, for inflammatory disorders, for atherosclerotic disorders and for male fertility control.

Further, it covers their use in combination with other anti cancer medications such as immunotherapeutics, targeted anti cancer agents, radiation or chemotherapy.

### DEFINITIONS

In case an asterix is used in a formula, like for instance in *-A-B or *-A-, this asterix indicates the bond towards the core of the compound.

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon or nitrogen or . . . atom. Commonly, it is possible for the number of optional substituents, when present, to be 1, 2, 3, 4 or 5, in particular 1, 2 or 3.

As used herein, the term “one or more”, e.g. in the definition of the substituents of the compounds of general formula (I) of the present invention, means “1, 2, 3, 4 or 5, particularly 1, 2, 3 or 4, more particularly 1, 2 or 3, even more particularly 1 or 2”.

When groups in the compounds according to the invention are substituted, it is possible for said groups to be mono-substituted or poly-substituted with substituent(s), unless otherwise specified. Within the scope of the present invention, the meanings of all groups which occur repeatedly are independent from one another. It is possible that groups in the compounds according to the invention are substituted with one, two or three identical or different substituents, particularly with one substituent.

As used herein, an oxo substituent represents an oxygen atom, which is bound to a carbon atom or to a sulfur atom via a double bond.

The term “ring substituent” means a substituent attached to an aromatic or nonaromatic ring which replaces an available hydrogen atom on the ring.

The term “comprising” when used in the specification includes “consisting of”.

If within the present text any item is referred to as “as mentioned herein”, it means that it may be mentioned anywhere in the present text.

The terms as mentioned in the present text have the following meanings:

The term “halogen atom” means a fluorine, chlorine, bromine or iodine atom, particularly a fluorine, chlorine or bromine atom.

The term “C₁-C₆-alkyl” means a linear or branched, saturated, monovalent hydrocarbon group having 1, 2, 3, 4, 5 or 6 carbon atoms, e.g. a methyl, ethyl, propyl, isopropyl, butyl, sec-butyl, isobutyl, tert-butyl, pentyl, isopentyl, 2-methylbutyl, 1-methylbutyl, 1-ethylpropyl, 1,2-dimethylpropyl, neo-pentyl, 1,1-dimethylpropyl, hexyl, 1-methylpentyl, 2-methylpentyl, 3-methylpentyl, 4-methylpentyl, 1-ethylbutyl, 2-ethylbutyl, 1,1-dimethylbutyl, 2,2-dimethylbutyl, 3,3-dimethylbutyl, 2,3-dimethylbutyl, 1,2-dimethylbutyl or 1,3-dimethylbutyl group, or an isomer thereof. Particularly, said group has 1, 2, 3 or 4 carbon atoms (“C₁-C₄-alkyl”), e.g. a methyl, ethyl, propyl, isopropyl, butyl, sec-butyl isobutyl, or tert-butyl group, more particularly 1, 2 or 3 carbon atoms (“C₁-C₃-alkyl”), e.g. a methyl, ethyl, n-propyl or isopropyl group.

The term “C₁-C₆-hydroxyalkyl” means a linear or branched, saturated, monovalent hydrocarbon group in which the term “C₁-C₆-alkyl” is defined supra, and in which 1, 2 or 3 hydrogen atoms are replaced with a hydroxy group, e.g. a hydroxymethyl, 1-hydroxyethyl, 2-hydroxyethyl, 1,2-dihydroxyethyl, 3-hydroxypropyl, 2-hydroxypropyl, 1-hydroxypropyl, 1-hydroxypropan-2-yl, 2-hydroxypropan-2-yl, 2,3-dihydroxypropyl, 1,3-dihydroxypropan-2-yl, 3-hydroxy-2-methyl-propyl, 2-hydroxy-2-methyl-propyl, 1-hydroxy-2-methyl-propyl group.

The term “C₁-C₆-haloalkyl” means a linear or branched, saturated, monovalent hydrocarbon group in which the term “C₁-C₆-alkyl” is as defined supra, and in which one or more of the hydrogen atoms are replaced, identically or differently, with a halogen atom. Particularly, said halogen atom is a fluorine atom. Said C₁-C₆-haloalkyl group is, for example, fluoromethyl, difluoromethyl, trifluoromethyl, 2-fluoroethyl, 2,2-difluoroethyl, 2,2,2-trifluoroethyl, pentafluoroethyl, 3,3,3-trifluoropropyl or 1,3-difluoropropan-2-yl.

Preference is given to perfluorinated alkyl radicals which are named as “perfluoro-C₁-Cₓ-alkyl-” wherein x is the maximum number of carbon atoms such as trifluoromethyl or 2,2,2-trifluoroethyl.

The term “C₁-C₆-cyanoalkyl” means a linear or branched, saturated, monovalent hydrocarbon group in which the term “C₁-C₆-alkyl” is as defined supra, and in which one or more of the hydrogen atoms are replaced, identically or differently, with a cyano group.

The term “C₁-C₆-alkoxy” means a linear or branched, saturated, monovalent group of formula (C₁-C₆-alkyl)-O—, in which the term “C₁-C₆-alkyl” is as defined supra, e.g. a methoxy, ethoxy, n-propoxy, isopropoxy, n-butoxy, sec-butoxy, isobutoxy, tert-butoxy, pentyloxy, isopentyloxy or n-hexyloxy group, or an isomer thereof.

The term “C₁-C₆-haloalkoxy” means a linear or branched, saturated, monovalent C₁-C₆-alkoxy group, as defined supra, in which one or more of the hydrogen atoms is replaced, identically or differently, with a halogen atom. Particularly, said halogen atom is a fluorine atom. Said C₁-C₆-haloalkoxy group is, for example, fluoromethoxy, difluoromethoxy, trifluoromethoxy or 2,2,2-trifluoroethoxy.

Preference is given to perfluorinated alkyl radicals which are named as “perfluoro-C₁-Cₓ-alkoxy-” wherein x is the maximum number of carbon atoms such as trifluoromethoxy and 2,2,2-trifluoroethoxy radicals.

The term “C₁-C₆-cyanoalkoxy” means a linear or branched, saturated, monovalent C₁-C₆-alkoxy group, as defined supra, in which one or more of the hydrogen atoms is replaced, identically or differently, with a cyano group.

Mono-(C₁-C₄)-alkylamino in the context of the invention means an amino group with one straight-chain or branched alkyl substituent which contains 1, 2, 3 or 4 carbon atoms, such as: methylamino, ethylamino, n-propylamino, isopropylamino, n-butylamino, and tert-butylamino, for example.

Di-(C₁-C₄)-alkylamino in the context of the invention means an amino group with two identical or different straight-chain or branched alkyl substituents which each contain 1, 2, 3 or 4 carbon atoms, such as: N,N-dimethylamino, N,N-diethylamino, N-ethyl-N-methylamino, N-methyl-N-n-propylamino, N-isopropyl-N-methylamino, N-isopropyl-N-n-propylamino, N,N-diisopropylamino, N-n-butyl-N-methylamino, and N-tert-butyl-N-methylamino, for example.

(C₁-C₄)-Alkylcarbonyl in the context of the invention means a straight-chain or branched alkyl group having 1, 2, 3 or 4 carbon atoms which is bound to the rest of the molecule via a carbonyl group [—C(═O)—], such as: acetyl, propionyl, n-butyryl, isobutyryl, n-pentanoyl, and pivaloyl, for example.

(C₁-C₄)-Alkylcarbonyloxy in the context of the invention means a straight-chain or branched alkyl group having 1, 2, 3 or 4 carbon atoms which is bound to the rest of the molecule via a carboxy group [—C(═O)—O—], such as: acetoxy (=acyloxy), propionyloxy, n-butyryloxy, isobutyryloxy, n-pentanoyloxy, and pivaloyloxy, for example.

Mono-(C₁-C₄)-alkylaminocarbonyl in the context of the invention means an amino group which is bound to the rest of the molecule via a carbonyl group [—C(═O)—] and which has one straight-chain or branched alkyl substituent having 1, 2, 3 or 4 carbon atoms, such as: methylaminocarbonyl, ethylaminocarbonyl, n-propylaminocarbonyl, isopropylaminocarbonyl, n-butylaminocarbonyl, and tert-butylaminocarbonyl, for example.

Di-(C₁-C₄)-alkylaminocarbonyl in the context of the invention means an amino group which is bound to the rest of the molecule via a carbonyl group [—C(═O)—] and which has two identical or different straight-chain or branched alkyl substituents having in each case 1, 2, 3 or 4 carbon atoms, such as: N,N-dimethylaminocarbonyl, N,N-diethylaminocarbonyl, N-ethyl-N-methylaminocarbonyl, N-methyl-N-n-propylaminocarbonyl, N-isopropyl-N-methylaminocarbonyl, N,N-diisopropylaminocarbonyl, N-n-butyl-N-methylaminocarbonyl, and N-tert-butyl-N-methylaminocarbonyl, for example.

Mono-(C₁-C₄)-alkylaminosulfonyl in the context of the invention means an amino group which is bound to the rest of the molecule via a sulfonyl group [—S(═O)₂-] and which has one straight-chain or branched alkyl substituent having 1, 2, 3 or 4 carbon atoms, such as: methylaminosulfonyl, ethylaminosulfonyl, n-propylaminosulfonyl, isopropylaminosulfonyl, n-butylamino sulfonyl, and tert-butylaminosulfonyl, for example.

Di-(C₁-C₄)-alkylaminosulfonyl in the context of the invention means an amino group which is bound to the rest of the molecule via a sulfonyl group [—S(═O)₂-] and which has two identical or different straight-chain or branched alkyl substituents having in each case 1, 2, 3 or 4 carbon atoms, such as: N,N-dimethylaminosulfonyl, N,N-diethylaminosulfonyl, N-ethyl-N-methylaminosulfonyl, N-methyl-N-n-propylaminosulfonyl, N-isopropyl-N-methylaminosulfonyl, N,N-diisopropylaminosulfonyl, N-n-butyl-N-methylaminosulfonyl, and N-tert-butyl-N-methylaminosulfonyl, for example.

The term “C₃-C₈-cycloalkyl” means a saturated, monovalent, mono- or bicyclic hydrocarbon ring which contains 3, 4, 5, 6, 7 or 8 carbon atoms (“C₃-C₅-cycloalkyl”). Said C₃-C₈-cycloalkyl group is for example, a monocyclic hydrocarbon ring, e.g. a cyclopropyl, cyclobutyl, cyclopentyl, cyclohexyl, cycloheptyl or cyclooctyl group, or a bicyclic hydrocarbon ring, e.g. a bicyclo[4.2.0]octyl or octahydropentalenyl.

The term “C₃-C₈-cycloalkoxy” means a saturated, monovalent, mono- or bicyclic group of formula (C₃-C₈-cycloalkyl)-O—, which contains 3, 4, 5, 6, 7 or 8 carbon atoms, in which the term “C₃-C₈-cycloalkyl” is defined supra, e.g. a cyclopropyloxy, cyclobutyloxy, cyclopentyloxy, cyclohexyloxy, cycloheptyloxy or cyclooctyloxy group.

The terms “4- to 7-membered heterocycloalkyl” and “4- to 6-membered heterocycloalkyl” mean a monocyclic, saturated or unsaturated heterocycle with 4, 5, 6 or 7 or, respectively, 4, 5 or 6 ring atoms in total, which contains one or two identical or different ring heteroatoms from the series N, O and S, it being possible for said heterocycloalkyl group to be attached to the rest of the molecule via any one of the carbon atoms or, if present, a nitrogen atom. A carbon atom may be substituted with an oxo group or the sulphur atom with one or two oxo groups to form a —C═O, —S(═O)— or —S(═O)₂-group in the ring.

Said heterocycloalkyl group, without being limited thereto, can be a 4-membered ring, such as azetidinyl, oxetanyl or thietanyl, for example; or a 5-membered ring, such as tetrahydrofuranyl, 1,3-dioxolanyl, thiolanyl, pyrrolidinyl, imidazolidinyl, pyrazolidinyl, 1,1-dioxidothiolanyl, 1,2-oxazolidinyl, 1,3-oxazolidinyl or 1,3-thiazolidinyl, for example; or a 6-membered ring, such as tetrahydropyranyl, tetrahydrothiopyranyl, piperidinyl, morpholinyl, dithianyl, thiomorpholinyl, piperazinyl, 1,3-dioxanyl, 1,4-dioxanyl or 1,2-oxazinanyl, for example, or a 7-membered ring, such as azepanyl, 1,4-diazepanyl or 1,4-oxazepanyl, for example.

Particularly, “4- to 6-membered heterocycloalkyl” means a 4- to 6-membered heterocycloalkyl as defined supra containing one ring nitrogen atom and optionally one further ring heteroatom from the series: N, O, S. More particularly, “5- or 6-membered heterocycloalkyl” means a monocyclic, saturated heterocycle with 5 or 6 ring atoms in total, containing one ring nitrogen atom and optionally one further ring heteroatom from the series: N, O.

The term “bridged heterocycloalkyl” means a bicyclic, saturated or unsaturated heterocycle with 7, 8, 9 or 10 ring atoms in total (=“bridged bicyclic 7- to 10-membered heterocycloalkyl”), in which the two rings share two common ring atoms which are not adjacent, which “bridged heterocycloalkyl” contains one or two identical or different ring heteroatoms from the series: N, O, S; it being possible for said bridged heterocycloalkyl group to be attached to the rest of the molecule via any one of the carbon atoms, except the spiro carbon atom, or, if present, a nitrogen atom. A carbon atom may be substituted with an oxo group or or the sulphur atom with one or two oxo groups to form a —C═O, —S(═O)— or —S(═O)₂-group in the ring.

Said bridged heterocycloalkyl group is, for example, azabicyclo[2.2.1]heptyl, oxazabicyclo[2.2.1]heptyl, thiazabicyclo[2.2.1]heptyl, diazabicyclo[2.2.1]heptyl, azabicyclo-[2.2.2]octyl, diazabicyclo[2.2.2]octyl, oxazabicyclo[2.2.2]octyl, thiazabicyclo[2.2.2]octyl, azabicyclo[3.2.1]octyl, diazabicyclo[3.2.1]octyl, oxazabicyclo[3.2.1]octyl, thiazabicyclo[3.2.1]octyl, azabicyclo[3.3.1]nonyl, diazabicyclo[3.3.1]nonyl, oxazabicyclo[3.3.1]nonyl, thiazabicyclo[3.3.1]nonyl, azabicyclo[4.2.1]nonyl, diazabicyclo[4.2.1]nonyl, oxazabicyclo[4.2.1]nonyl, thiazabicyclo[4.2.1]nonyl, azabicyclo[3.3.2]decyl, diazabicyclo[3.3.2]decyl, oxazabicyclo[3.3.2]decyl, thiazabicyclo[3.3.2]decyl or azabicyclo[4.2.2]decyl.

The term “heteroaryl” means a monovalent, monocyclic, bicyclic or tricyclic aromatic ring having 5, 6, 8, 9, 10, 11, 12, 13 or 14 ring atoms (a “5- to 14-membered heteroaryl” group), particularly 5, 6, 9 or 10 ring atoms, which contains at least one ring heteroatom and optionally one, two or three further ring heteroatoms from the series: N, O and/or S, and which is bound via a ring carbon atom or optionally via a ring nitrogen atom (if allowed by valency).

Said heteroaryl group can be a 5-membered heteroaryl group, such as, for example, thienyl, furanyl, pyrrolyl, oxazolyl, thiazolyl, imidazolyl, pyrazolyl, isoxazolyl, isothiazolyl, oxadiazolyl, triazolyl, thiadiazolyl or tetrazolyl; or a 6-membered heteroaryl group, such as, for example, pyridinyl, pyridazinyl, pyrimidinyl, pyrazinyl or triazinyl; or a tricyclic heteroaryl group, such as, for example, carbazolyl, acridinyl or phenazinyl; or a 9-membered heteroaryl group, such as, for example, benzofuranyl, benzothienyl, benzoxazolyl, benzisoxazolyl, benzimidazolyl, benzothiazolyl, benzotriazolyl, indazolyl, indolyl, isoindolyl, indolizinyl or purinyl; or a 10-membered heteroaryl group, such as, for example, quinolinyl, quinazolinyl, isoquinolinyl, cinnolinyl, phthalazinyl, quinoxalinyl or pteridinyl.

In general, and unless otherwise mentioned, the heteroaryl or heteroarylene groups include all possible isomeric forms thereof, e.g.: tautomers and positional isomers with respect to the point of linkage to the rest of the molecule. Thus, for some illustrative non-restricting examples, the term pyridinyl includes pyridin-2-yl, pyridin-3-yl and pyridin-4-yl; or the term thienyl includes thien-2-yl and thien-3-yl.

Particularly, the heteroaryl group is a 5-membered heteroaryl group, such as, for example, thienyl, furanyl, pyrrolyl, oxazolyl, thiazolyl, imidazolyl, pyrazolyl, isoxazolyl, isothiazolyl, oxadiazolyl, triazolyl, thiadiazolyl or tetrazolyl; or a 6-membered heteroaryl group group such as, for example, pyridinyl (=pyridyl), pyridazinyl, pyrimidinyl, pyrazinyl or triazinyl.

The term “C₁-C₆”, as used in the present text, e.g. in the context of the definition of “C₁-C₆-alkyl”, “C₁-C₆-haloalkyl”, “C₁-C₆-hydroxyalkyl”, “C₁-C₆-alkoxy” or “C₁-C₆-haloalkoxy” means an alkyl group having a finite number of carbon atoms of 1 to 6, i.e. 1, 2, 3, 4, 5 or 6 carbon atoms. Further, as used herein, the term “C₃-C₈”, as used in the present text, e.g. in the context of the definition of “C₃-C₅-cycloalkyl”, means a cycloalkyl group having a finite number of carbon atoms of 3 to 8, i.e. 3, 4, 5, 6, 7 or 8 carbon atoms.

When a range of values is given, said range encompasses each value and sub-range within said range.

For example:
“C₁-C₆” encompasses C₁, C₂, C₃, C₄, C₅, C₆, C₁-C₄, C₁-C₅, C₁-C₄, C₁-C₃, C₁-C₂, C₂- C₆, C₂-C₅, C₂-C₄, C₂-C₃, C₃-C₆, C₃-C₅, C₃-C₄, C₄-C₆, C₄-C₅, and C₅-C₆;
“C₂-C₆” encompasses C₂, C₃, C₄, C₅, C₆, C₂-C₆, C₂-C₅, C₂-C₄, C₂-C₃, C₃-C₆, C₃-C₅, C₃-C₄, C₄-C₆, C₄-C₅, and C₅-C₆;
“C₃-C₁₀” encompasses C₃, C₄, C₅, C₆, C₇, C₈, C₉, C₁₀, C₃-C₁₀, C₃-C₉, C₃-C₈, C₃-C₇, C₃-C₆, C₃-C₅, C₃-C₄, C₄-C₁₀, C₄-C₉, C₄-C₈, C₄-C₇, C₄-C₆, C₄-C₅, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉ and C₉-C₁₀;
“C₃-C₈” encompasses C₃, C₄, C₅, C₆, C₇, C₅, C₃-C₈, C₃-C₇, C₃-C₆, C₃-C₅, C₃-C₄, C₄- C₈, C₄-C₇, C₄-C₆, C₄-C₅, C₅-C₈, C₅-C₇, C₅-C₆, C₆-C₈, C₆-C₇ and C₇-C₈;
“C₃-C₆” encompasses C₃, C₄, C₅, C₆, C₃-C₆, C₃-C₅, C₃-C₄, C₄-C₆, C₄-C₅, and C₅-C₆;
“C₄-C₈” encompasses C₄, C₅, C₆, C₇, C₈, C₄-C₈, C₄-C₇, C₄-C₆, C₄-C₅, C₅-C₈, C₅-C₇, C₅-C₆, C₆-C₈, C₆-C₇ and C₇-C₈;
“C₄-C₇” encompasses C₄, C₅, C₆, C₇, C₄-C₇, C₄-C₆, C₄-C₅, C₅-C₇, C₅-C₆ and C₆-C₇;
“C₄-C₆” encompasses C₄, C₅, C₆, C₄-C₆, C₄-C₅ and C₅-C₆;
“C₅-C₁₀” encompasses C₅, C₆, C₇, C₈, C₉, C₁₀, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉ and C₉-C₁₀;
“C₆-C₁₀” encompasses C₆, C₇, C₈, C₉, C₁₀, C₆-C₁₀, C₆-C₉, C₆-C₈, C₆-C₇, C₇-C₁₀, C₇-C₉, C₇-C₈, C₈-C₁₀, C₈-C₉ and C₉-C₁₀.

As used herein, the term “leaving group” means an atom or a group of atoms that is displaced in a chemical reaction as stable species taking with it the bonding electrons. In particular, such a leaving group is selected from the group comprising: halide, in particular fluoride, chloride, bromide or iodide, (methylsulfonyl)oxy, [(trifluoromethyl)sulfonyl]oxy, [(nonafluorobutyl)sulfonyl]oxy, (phenylsulfonyl)oxy, [(4-methylphenyl)sulfonyl]oxy, [(4-bromophenyl)sulfonyl]oxy, [(4-nitrophenyl)sulfonyl]oxy, [(2-nitrophenyl)sulfonyl]oxy, [(4-isopropylphenyl)sulfonyl]oxy, [(2,4,6-triisopropylphenyl)sulfonyl]oxy, [(2,4,6-trimethylphenyl)sulfonyl]oxy, [(4-tert-butyl-phenyl)sulfonyl]oxy and [(4-methoxyphenyl)sulfonyl]oxy.

Where the plural form of the word compounds, salts, polymorphs, hydrates, solvates and the like, is used herein, this is taken to mean also a single compound, salt, polymorph, isomer, hydrate, solvate or the like.

By “stable compound’ or “stable structure” is meant a compound that is sufficiently robust to survive isolation to a useful degree of purity from a reaction mixture, and formulation into an efficacious therapeutic agent.

The compounds of the present invention optionally contain one or more asymmetric centres, depending upon the location and nature of the various substituents desired. It is possible that one or more asymmetric carbon atoms are present in the (R) or (S) configuration, which can result in racemic mixtures in the case of a single asymmetric centre, and in diastereomeric mixtures in the case of multiple asymmetric centres. In certain instances, it is possible that asymmetry also be present due to restricted rotation about a given bond, for example, the central bond adjoining two substituted aromatic rings of the specified compounds.

Preferred compounds are those which produce the more desirable biological activity. Separated, pure or partially purified isomers and stereoisomers or racemic or diastereomeric mixtures of the compounds of the present invention are also included within the scope of the present invention. The purification and the separation of such materials can be accomplished by standard techniques known in the art.

Preferred isomers are those which produce the more desirable biological activity. These separated, pure or partially purified isomers or racemic mixtures of the compounds of this invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or freactional crystallisation. The optically active bases or acids are then liberated from the separated diastereomeric salts. A different process for separation of optical isomers involves the use of chiral chromatography (e.g., HPLC columns using a chiral phase), with or without conventional derivatisation, optimally chosen to maximise the separation of the enantiomers. Suitable HPLC columns using a chiral phase are commercially available, such as those manufactured by Daicel, e.g., Chiracel OD and Chiracel OJ, for example, among many others, which are all routinely selectable. Enzymatic separations, with or without derivatisation, are also useful. The optically active compounds of the present invention can likewise be obtained by chiral syntheses utilizing optically active starting materials.

In order to distinguish different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. (R)- or (S)-isomers, in any ratio. Isolation of a single stereoisomer, e.g. a single enantiomer or a single diastereomer, of a compound of the present invention is achieved by any suitable state of the art method, such as chromatography, especially chiral chromatography, for example.

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

The present invention also covers useful forms of the compounds of the present invention, such as metabolites, hydrates, solvates, prodrugs, salts, in particular pharmaceutically acceptable salts, and/or co-precipitates.

The compounds of the present invention can exist as a hydrate, or as a solvate, wherein the compounds of the present invention contain polar solvents, in particular water, methanol or ethanol for example, as structural element of the crystal lattice of the compounds. It is possible for the amount of polar solvents, in particular water, to exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, it is possible for the compounds of the present invention to exist in free form, e.g. as a free base, or as a free acid, or as a zwitterion, or to exist in the form of a salt. Said salt may be any salt, either an organic or inorganic addition salt, particularly any pharmaceutically acceptable organic or inorganic addition salt, which is customarily used in pharmacy, or which is used, for example, for isolating or purifying the compounds of the present invention.

The term “pharmaceutically acceptable salt” refers to an inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, or “mineral acid”, such as hydrochloric, hydrobromic, hydroiodic, sulfuric, sulfamic, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, 3-phenylpropionic, pivalic, 2-hydroxyethanesulfonic, itaconic, trifluoromethanesulfonic, dodecylsulfuric, ethanesulfonic, benzenesulfonic, para-toluenesulfonic, methanesulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium, magnesium or strontium salt, or an aluminium or a zinc salt, or an ammonium salt derived from ammonia or from an organic primary, secondary or tertiary amine having 1 to 20 carbon atoms, such as ethylamine, diethylamine, triethylamine, ethyldiisopropylamine, monoethanolamine, diethanolamine, triethanolamine, dicyclohexylamine, dimethylaminoethanol, diethylaminoethanol, tris(hydroxymethyl)aminomethane, procaine, dibenzylamine, N-methylmorpholine, arginine, lysine, 1,2-ethylenediamine, N-methylpiperidine, N-methyl-glucamine, N,N-dimethyl-glucamine, N-ethyl-glucamine, 1,6-hexanediamine, glucosamine, sarcosine, serinol, 2-amino-1,3-propanediol, 3-amino-1,2-propanediol, 4-amino-1,2,3-butanetriol, or a salt with a quarternary ammonium ion having 1 to 20 carbon atoms, such as tetramethylammonium, tetraethylammonium, tetra(n-propyl)ammonium, tetra(n-butyl)ammonium, N-benzyl-N,N,N-trimethylammonium, choline or benzalkonium.

Those skilled in the art will further recognise that it is possible for acid addition salts of the claimed compounds to be prepared by reaction of the compounds with the appropriate inorganic or organic acid via any of a number of known methods. Alternatively, alkali and alkaline earth metal salts of acidic compounds of the present invention are prepared by reacting the compounds of the present invention with the appropriate base via a variety of known methods.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown. Unless specified otherwise, suffixes to chemical names or structural formulae relating to salts, such as “hydrochloride”, “trifluoroacetate”, “sodium salt”, or “x HCl”, “x CF₃COOH”, “x Na*”, for example, mean a salt form, the stoichiometry of which salt form not being specified.

This applies analogously to cases in which synthesis intermediates or example compounds or salts thereof have been obtained, by the preparation and/or purification processes described, as solvates, such as hydrates, with (if defined) unknown stoichiometric composition.

Furthermore, the present invention includes all possible crystalline forms, or polymorphs, of the compounds of the present invention, either as single polymorph, or as a mixture of more than one polymorph, in any ratio.

Moreover, the present invention also includes prodrugs of the compounds according to the invention. The term “prodrugs” here designates compounds which themselves can be biologically active or inactive, but are converted (for example metabolically or hydrolytically) into compounds according to the invention during their residence time in the body.

The invention further includes all possible crystallized and polymorphic forms of the inventive compounds, whereby the polymorphs are existing either as a single polymorph form or are existing as a mixture of several polymorphs in all concentrations.

The invention further includes all possible cyclodextrin clathrates, i.e alpha-, beta-, or gamma-cyclodextrins, hydroxypropyl-beta-cyclodextrins, methylbetacyclodextrins.

The invention also covers the following embodiments:

### Embodiment 2

### Compound of Formula (Ia)

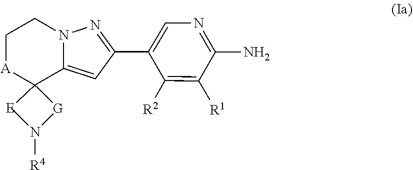 [0206] wherein
A is selected from a single bond, —CH₂— or —O—;
E is —(CH₂)ₙ— with n=1 or 2;
G is —(CH₂)— and with the condition, that the cyclic substructure comprising E-N-G is a 4 to 5 membered heterocycloalkyl;
R¹ is selected from
   —NO₂; —CN;
   C₁₋₄-alkyl optionally substituted by —OH or 1 to 3-F;
   cyclopropyl;
   4 to 6 membered heterocycloalkyl or heterocycloalkenyl, both with 1-2 nitrogen atoms, but always linked via a carbon atom to the phenyl group of compounds of formula (I) and both optionally substituted by C₁₋₄-alkyl;
   phenyl optionally substituted by halogen, —CN, C₁₋₄-alkyl, C₁₋₄-haloalkyl, C₃₋₆-cycloalkyl, O—C₁₋₄-alkyl, O—C₃₋₆-cycloalky, C₁₋₃-alkinyl, —C(O)—R_{d}, SR_{d}, SOR_{d}, SO₂R_{d}, SONR_{d}, SON(H)R_{d}, —SO₂—NH₂, —SO₂—N(H)—R_{d} or —SO₂—N(R_{d})₂, wherein R_{d} is selected from methyl, ethyl, cyclopropyl, isopropyl and tert·butyl;
   5 to 6 membered heteroaryl with 1 or 2 heteroatoms selected from N or S and optionally substituted by halogen, —CN, C₁₋₄-alkyl, C₁₋₄-haloalkyl, C₃₋₆-cycloalkyl, O—C₁₋₄-alkyl, O—C₃₋₆-cycloalkyl, C₁₋₃-alkinyl, SR_{d}, SOR_{d}, SO₂R_{d}, SONR_{d} or SON(H)R_{d} wherein R_{d} is selected from methyl, ethyl, cyclopropyl, isopropyl and tert·butyl;
   —O—CH₃; —O—CH(CH₃)₂; —O—CHF₂; —O—CF₃;

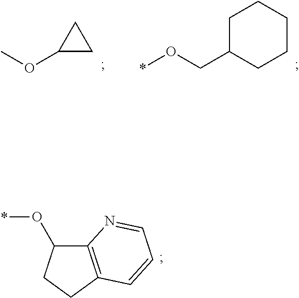

—O—CHRₐ-phenyl optionally substituted by one or two of the following substituents: halogen, —CN, C₁₋₄-alkyl optionally substituted by one or more —F or —SO₂—CH₃ and with Rₐ selected from —H, —F or —CH₃, —CF₃, —CH₂—CH₃, —CH(CH₃)₂ or -cyclopropyl;
—O—CHRₐ-6 membered heteroaryl optionally substituted one or two of the following substituents: halogen, —CN, C₁₋₄-alkyl optionally substituted by one or more —F or —SO₂—CH₃ and with Rₐ selected from —H, —F or —CH₃, —CF₃, —CH₂—CH₃, —CH(CH₃)₂ or -cyclopropyl;
COORₐ with Rₐ selected from —H or —CH₃;
C(═O)-methyl, optionally substituted by 1 to 3-F;
—C(═O)—NR_{b}R_{c} wherein R_{b} is selected from —H, —CH₃ or —CH₂—CH₃ and wherein R_{c} is selected from —H, —CH₃, —CH₂—CH₃, cyclohexyl, phenyl or 5 to 6 membered heteroaryl, all optionally substituted by one or two halogen, —CN, —SO₂—CH₃ or C₁₋₄-alkyl, optionally substituted one or more times with halogen;
S—CR_{f}R_{g}Rₕ, wherein R_{f} is selected from —H or —F, wherein R_{g} is selected from —H, —F or —CH₃ and wherein Rₕ is selected from H, F, —CH₃ or C₅₋₆-cycloalkyl or phenyl;

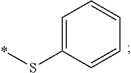 [SMILES: *Sc1ccccc1]

R² is selected from —H, or
R⁴ is selected from
   C₁₋₅-alkyl or C₃₋₆-cycloalkyl, both optionally substituted one or independently more times by
      —F, —Cl, —Br, —CN;
      —OH, —ORᵢ wherein Rᵢ is selected from C₁₋₄-alkyl or C₃₋₆Cycloalkyl),
      —CF₃, —CHF₂, —CH₂F, or —CH₂—CF₃;
      —NRⱼRₖ wherein Rⱼ and Rₖ are independently selected from —H and C₁-C₄-alkyl,
      —CH₂—O—CH₃ or
      C₃₋₄ cycloalkyl or 4 membered heterocycloalkyl, both optionally substituted by —F or —CH₃;
      phenyl or 5 to 6 membered heteroaryl, both optionally substituted one or more times by
         halogen, —CN,
         —CF₃, —CHF₂, —CH₂F,
         C₁₋₄-alkyl optionally substituted with one or more halogen;
         C₃₋₆-cycloalkyl;
         O—C₁₋₄-alkyl,
         O—C₃₋₆-cycloalky,
         C₁₋₃-alkinyl,
         —NRⱼRₖ wherein Rⱼ and Rₖ are independently selected from —H and C₁-C₄-alkyl
         SR_{d}, SOR_{d}, SO₂R_{d}, SONR_{d} or SON(H)R_{d} wherein R_{d} is selected from methyl, ethyl, cyclopropyl, isopropyl and tert·butyl;
   C(═O)—Rᵢ in which Rᵢ is selected from
      C₁₋₄-alkyl, C₃₋₆-cycloalkyl or C₃₋₆-heterocycloalkyl, both optionally substituted one or more times by
         —F, —Cl, —Br, —CN;
         —OH, —ORᵢ with Rᵢ selected from C₁₋₄-alkyl or C₃₋₆Cycloalkyl), —CF₃, —CHF₂, —CH₂F;
         —NRⱼRₖ wherein Rⱼ and Rₖ are independently selected from —H and C₁-C₄-alkyl
         phenyl or 5 to 6 membered heteroaryl, both optionally substituted one or more times by
         halogen, —CN,
         —CF₃, —CHF₂, —CH₂F,
         C₁₋₄-alkyl optionally substituted with one or more halogen;
         C₃₋₆-cycloalkyl;
         O—C₁₋₄-alkyl,
         O—C₃₋₆-cycloalky,
         C₁₋₃-alkinyl,
         —NRⱼRₖ wherein Rⱼ and Rₖ are independently selected from —H and C₁-C₄-alkyl
         SR_{d}, SOR_{d}, SO₂R_{d}, SONR_{d} or SON(H)R_{d} wherein R_{d} is selected from methyl, ethyl, cyclopropyl, isopropyl and tert·butyl;
      phenyl or 5 to 6 membered heteroaryl, both optionally substituted one or more times by
         halogen, —CN,
         —CF₃, —CHF₂, —CH₂F,
         C₁₋₄-alkyl optionally substituted with one or more halogen;
         C₃₋₆-cycloalkyl;
         O—C₁₋₄-alkyl,
         O—C₃₋₆-cycloalky,
         C₁₋₃-alkinyl,
         —NRⱼRₖ wherein Rⱼ and Rₖ are independently selected from —H and C₁-C₄-alkyl
         SR_{d}, SOR_{d}, SO₂R_{d}, SONR_{d} or SON(H)R_{d} wherein R_{d} is selected from methyl, ethyl, cyclopropyl, isopropyl and tert·butyl;
   C(═O)—NRₒRₚ wherein Rₒ and Rₚ are independently selected from
      H
      C₁₋₄-alkyl or C₃₋₆-cycloalkyl, both optionally substituted one or more times by
         —F, —Cl, —Br, —CN;
         —OH, —ORᵢ with Rᵢ selected from C₁₋₄-alkyl or C₃₋₆-cycloalkyl, —CF₃, —CHF₂, —CH₂F;
         —NRⱼRₖ wherein Rⱼ and Rₖ are independently selected from —H and C₁-C₄-alkyl
         phenyl or 5 to 6 membered heteroaryl, both optionally substituted one or more times by
         halogen, —CN,
         —CF₃, —CHF₂, —CH₂F,
         C₁₋₄-alkyl optionally substituted with one or more halogen;
         C₃₋₆-cycloalkyl;
         O—C₁₋₄-alkyl,
         O—C₃₋₆-cycloalky,
         C₁₋₃-alkinyl,
         —NRⱼRₖ wherein Rⱼ and Rₖ are independently selected from —H and C₁-C₄-alkyl
         SR_{d}, SOR_{d}, SO₂R_{d}, SONR_{d} or SON(H)R_{d} wherein R_{d} is selected from methyl, ethyl, cyclopropyl, isopropyl and tert·butyl;
      phenyl or 5 to 6 membered heteroaryl, both optionally substituted one or more times by
         halogen, —CN,
         —CF₃, —CHF₂, —CH₂F,
         C₁₋₄-alkyl optionally substituted with one or more halogen;
         C₃₋₆-cycloalkyl;
         O—C₁₋₄-alkyl,
         O—C₃₋₆-cycloalky,
         C₁₋₃-alkinyl,
         —NRⱼRₖ wherein Rⱼ and Rₖ are independently selected from —H and C₁-C₄-alkyl
         SR_{d}, SOR_{d}, SO₂R_{d}, SONR_{d} or SON(H)R_{d} wherein R_{d} is selected from methyl, ethyl, cyclopropyl, isopropyl and tert·butyl;

or a stereoisomer, a tautomer, an N-oxide, a hydrate, a solvate, or a salt thereof, or a mixture of same.

### Embodiment 3

Compounds of formula (I) according to embodiment 1, wherein A is selected from a single bond or —O— or a stereoisomer, a tautomer, an N-oxide, a hydrate, a solvate, or a salt thereof, or a mixture of same.

### Embodiment 4

Compounds of formula (I) according to embodiment 1, wherein E is —CH₂—CH₂— or a stereoisomer, a tautomer, an N-oxide, a hydrate, a solvate, or a salt thereof, or a mixture of same.

### Embodiment 5

Compounds of formula (I) according to embodiment 1, wherein Q is C—R² or a stereoisomer, a tautomer, an N-oxide, a hydrate, a solvate, or a salt thereof, or a mixture of same.

### Embodiment 6

Compounds of formula (I) according to embodiment 1, wherein Q is N or a stereoisomer, a tautomer, an N-oxide, a hydrate, a solvate, or a salt thereof, or a mixture of same.

### Embodiment 7

Compounds of formula (I) according to embodiment 1, wherein R₄ is

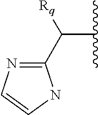

and R_{q} is selected from H, —CH₃, —CH₂—CH₃, —CH(CH₃)₂, —CH₂—O—CH₃, —CF₃, —CHF₂, —CH₂F, —CH₂—CF₃, cyclopropyl or cyclobutyl or a stereoisomer, a tautomer, an N-oxide, a hydrate, a solvate, or a salt thereof, or a mixture of same.

### Embodiment 8

Compounds of formula (I) according to embodiment 1, wherein R₄ is selected from C(═O)—NRₒRₚ wherein Rₒ and Rₚ are independently selected from
H
C₁₋₄-alkyl or C₃₋₆-cycloalkyl, both optionally substituted one or more times by
   —F, —Cl, —Br, —CN;
   —OH, —ORᵢ with Rᵢ selected from C₁₋₄-alkyl or C₃₋₆-cycloalkyl, —CF₃, —CHF₂, —CH₂F;
   —NRⱼRₖ wherein Rⱼ and Rₖ are independently selected from —H and C₁-C₄-alkyl
   phenyl or 5 to 6 membered heteroaryl, both optionally substituted one or more times by
      halogen, —CN,
      —CF₃, —CHF₂, —CH₂F,
      C₁₋₄-alkyl optionally substituted with one or more halogen;
      C₃₋₆-cycloalkyl;
      O—C₁₋₄-alkyl,
      O—C₃₋₆-cycloalky,
      C₁₋₃-alkinyl,
      —NRⱼRₖ wherein Rⱼ and Rₖ are independently selected from —H and C₁-C₄-alkyl
      SR_{d}, SOR_{d}, SO₂R_{d}, SONR_{d} or SON(H)R_{d} wherein R_{d} is selected from methyl, ethyl, cyclopropyl, isopropyl and tert·butyl;

or a stereoisomer, a tautomer, an N-oxide, a hydrate, a solvate, or a salt thereof, or a mixture of same.

### Embodiment 9

Compound according to embodiment 1, which is selected from:
(rac)-2′-[6-amino-5-(trifluoromethyl)pyridin-3-yl]-N-[1-(2-chlorophenyl)cyclobutyl]-5′,6′-dihydrospiro[pyrrolidine-3,4′-pyrrolo[1,2-b]pyrazole]-1-carboxamide
(rac)-2-[6-amino-5-(trifluoromethyl)pyridin-3-yl]-N-[2-(2-fluorophenyl)propan-2-yl]-6,7-dihydrospiro[pyrazolo[5,1-c][1,4]oxazine-4,3′-pyrrolidine]-1′-carboxamide
(rac)-2-[6-amino-5-(trifluoromethyl)pyridin-3-yl]-N-(1-phenylcyclobutyl)-6,7-dihydrospiro[pyrazolo[5,1-c][1,4]oxazine-4,3′-pyrrolidine]-1′-carboxamide
(rac)-2′-[6-amino-5-(trifluoromethyl)pyridin-3-yl]-N-[2-(4-fluorophenyl)propan-2-yl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[2-(2-chlorophenyl)propan-2-yl]-5′,6′-dihydrospiro[pyrrolidine-3,4′-pyrrolo[1,2-b]pyrazole]-1-carboxamide
(rac)-2-[6-amino-5-(trifluoromethyl)pyridin-3-yl]-N-[(1R)-1-(2-chloro-4-cyanophenyl)ethyl]-6,7-dihydrospiro[pyrazolo[5,1-c][1,4]oxazine-4,3′-pyrrolidine]-1′-carboxamide
(rac)-2′-[6-amino-5-(trifluoromethyl)pyridin-3-yl]-N-[(1R)-1-(2-chloro-4-cyanophenyl)ethyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[(1S)-1-(3-chloropyridin-4-yl)ethyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[2-(3-fluorophenyl)propan-2-yl]-5′,6′-dihydrospiro[pyrrolidine-3,4′-pyrrolo[1,2-b]pyrazole]-1-carboxamide
(rac)-2-[6-amino-5-(trifluoromethyl)pyridin-3-yl]-N-[1-(pyridin-4-yl)cyclobutyl]-6,7-dihydrospiro[pyrazolo[5,1-c][1,4]oxazine-4,3′-pyrrolidine]-1′-carboxamide
(rac)-2′-[6-amino-5-(trifluoromethyl)pyridin-3-yl]-N-[2-(3-chloropyridin-4-yl)propan-2-yl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-(1-phenylcyclobutyl)-5′,6′-dihydrospiro[pyrrolidine-3,4′-pyrrolo[1,2-b]pyrazole]-1-carboxamide
(rac)-2-[6-amino-5-(trifluoromethyl)pyridin-3-yl]-N-[2-(pyridin-2-yl)propan-2-yl]-6,7-dihydrospiro[pyrazolo[5,1-c][1,4]oxazine-4,3′-pyrrolidine]-1′-carboxamide
(rac)-2′-(5-acetyl-6-aminopyridin-3-yl)-N-(1-phenylcyclobutyl)-5′,6′-dihydrospiro[pyrrolidine-3,4′-pyrrolo[1,2-b]pyrazole]-1-carboxamide
(rac)-2-(6-amino-5-cyanopyridin-3-yl)-N-[1-(2-chlorophenyl)cyclobutyl]-6,7-dihydrospiro[pyrazolo[5,1-c][1,4]oxazine-4,3′-pyrrolidine]-1′-carboxamide
(rac)-2′-(6-amino-5-cyanopyridin-3-yl)-N-[1-(2-chlorophenyl)cyclobutyl]-5′,6′-dihydrospiro[pyrrolidine-3,4′-pyrrolo[1,2-b]pyrazole]-1-carboxamide
(rac)-2-[6-amino-5-(trifluoromethyl)pyridin-3-yl]-N-[2-(3-chloropyridin-4-yl)propan-2-yl]-6,7-dihydrospiro[pyrazolo[5,1-c][1,4]oxazine-4,3′-pyrrolidine]-1′-carboxamide
(rac)-2-(6-amino-5-cyanopyridin-3-yl)-N-[1-(pyridin-4-yl)cyclobutyl]-6,7-dihydrospiro[pyrazolo[5,1-c][1,4]oxazine-4,3′-pyrrolidine]-1′-carboxamide
(rac)-2-[6-amino-5-(trifluoromethyl)pyridin-3-yl]-N-[1-(2-fluorophenyl)cyclobutyl]-6,7-dihydrospiro[pyrazolo[5,1-c][1,4]oxazine-4,3′-pyrrolidine]-1′-carboxamide
(rac)-2′-(6-amino-5-cyanopyridin-3-yl)-N-(1-phenylcyclobutyl)-5′,6′-dihydrospiro[pyrrolidine-3,4′-pyrrolo[1,2-b]pyrazole]-1-carboxamide
(rac)-2-(6-amino-5-cyanopyridin-3-yl)-N-[1-(4-fluorophenyl)cyclobutyl]-6,7-dihydrospiro[pyrazolo[5,1-c][1,4]oxazine-4,3′-pyrrolidine]-1′-carboxamide
(rac)-2′-(6-amino-5-cyanopyridin-3-yl)-N-[1-(4-fluorophenyl)cyclobutyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[1-(2-fluorophenyl)cyclobutyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[1-(3-fluorophenyl)cyclobutyl]-5′,6′-dihydrospiro[pyrrolidine-3,4′-pyrrolo[1,2-b]pyrazole]-1-carboxamide
(rac)-2′-(6-amino-5-cyanopyridin-3-yl)-N-[2-(3-chloropyridin-4-yl)propan-2-yl]-5′,6′-dihydrospiro[pyrrolidine-3,4′-pyrrolo[1,2-b]pyrazole]-1-carboxamide
(rac)-2′-(6-amino-5-cyanopyridin-3-yl)-N-[1-(pyridin-4-yl)cyclobutyl]-5′,6′-dihydrospiro[pyrrolidine-3,4′-pyrrolo[1,2-b]pyrazole]-1-carboxamide
(rac)-2-(6-amino-5-cyanopyridin-3-yl)-N-(1-phenylcyclobutyl)-6,7-dihydrospiro[pyrazolo[5,1-c][1,4]oxazine-4,3′-pyrrolidine]-1′-carboxamide
(rac)-2-(6-amino-5-cyanopyridin-3-yl)-N-[2-(4-fluorophenyl)propan-2-yl]-6,7-dihydrospiro[pyrazolo[5,1-c][1,4]oxazine-4,3′-pyrrolidine]-1′-carboxamide
(rac)-2′-[6-amino-5-(trifluoromethyl)pyridin-3-yl]-N-[1-(pyridin-4-yl)cyclobutyl]-5′,6′-dihydrospiro[pyrrolidine-3,4′-pyrrolo[1,2-b]pyrazole]-1-carboxamide
(rac)-2-[6-amino-5-(trifluoromethyl)pyridin-3-yl]-N-[1-(3-fluorophenyl)cyclobutyl]-6,7-dihydrospiro[pyrazolo[5,1-c][1,4]oxazine-4,3′-pyrrolidine]-1′-carboxamide
2′-[6-amino-5-(trifluoromethyl)pyridin-3-yl]-N-{(1R)-1-[3-(trifluoromethyl)phenyl]ethyl}-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[2-(3-fluoropyridin-2-yl)propan-2-yl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[2-(pyridin-4-yl)propan-2-yl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[2-(2-fluorophenyl)propan-2-yl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1R)-1-(3-fluorophenyl)ethyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[2-(pyridin-3-yl)propan-2-yl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[1-(pyridin-3-yl)cyclobutyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1R)-1-(4-fluorophenyl)ethyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1R)-1-phenylpropyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-{(1S)-1-[3-(trifluoromethyl)phenyl]ethyl}-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1R)-1-phenylethyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[2-(pyridin-2-yl)propan-2-yl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1R)-1-(2-cyanophenyl)ethyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[1-(1,3,5-trimethyl-1H-pyrazol-4-yl)ethyl]-5′,6′-dihydrospiro[pyrrolidine-3,4′-pyrrolo[1,2-b]pyrazole]-1-carboxamide (diastereomeric mixture)
2′-[6-amino-5-(trifluoromethyl)pyridin-3-yl]-N-[(1R)-2,3-dihydro-1H-inden-1-yl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1R)-1-(2-cyanopyridin-4-yl)ethyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[1-(1-ethyl-3,5-dimethyl-1H-pyrazol-4-yl)ethyl]-5′,6′-dihydrospiro[pyrrolidine-3,4′-pyrrolo[1,2-b]pyrazole]-1-carboxamide (diastereometric mixture)
(rac)-2′-[6-amino-5-(trifluoromethyl)pyridin-3-yl]-N-[1-(pyridin-2-yl)cyclobutyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[1-(1,3,4-trimethyl-1H-pyrazol-5-yl)ethyl]-5′,6′-dihydrospiro[pyrrolidine-3,4′-pyrrolo[1,2-b]pyrazole]-1-carboxamide (diastereometric mixture)
2′-[6-amino-5-(trifluoromethyl)pyridin-3-yl]-N-[(1R)-1-(4-cyanopyridin-2-yl)ethyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1R)-1-(pyridin-3-yl)ethyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[2-(1-methyl-1H-1,2,3-triazol-4-yl)propan-2-yl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-{[3-(trifluoromethyl)phenyl]methyl}-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[1-(1-phenyl-1H-pyrazol-4-yl)ethyl]-5′,6′-dihydrospiro[pyrrolidine-3,4′-pyrrolo[1,2-b]pyrazole]-1-carboxamide (diastereometric mixture)
2′-[6-amino-5-(trifluoromethyl)pyridin-3-yl]-N-[(3S)-2,3-dihydro-1-benzofuran-3-yl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1R)-1-(6-cyanopyridin-3-yl)ethyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-{(1S)-1-[1-methyl-3-(trifluoromethyl)-1H-pyrazol-4-yl]ethyl}-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1R)-1-(pyridin-2-yl)ethyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-benzyl-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1S)-1-phenylethyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1S)-1-(1,2-oxazol-3-yl)ethyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1S)-1-(1,5-dimethyl-1H-pyrazol-4-yl)ethyl]-5′,6′-dihydrospiro[pyrrolidine-3,4′-pyrrolo[1,2-b]pyrazole]-1-carboxamide (diastereomeric mixture)
(rac)-2′-[6-amino-5-(trifluoromethyl)pyridin-3-yl]-N-[2-(oxan-4-yl)propan-2-yl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1R)-1-(pyridin-4-yl)ethyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1S)-1-(1-ethyl-5-methyl-1H-pyrazol-4-yl)ethyl]-5′,6′-dihydrospiro[pyrrolidine-3,4′-pyrrolo[1,2-b]pyrazole]-1-carboxamide (diastereomeric mixture)
(rac)-2′-[6-amino-5-(trifluoromethyl)pyridin-3-yl]-N-[(pyridin-3-yl)methyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1S)-2-methoxy-1-phenylethyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-{[3-(trifluoromethyl)bicyclo[1.1.1]pentan-1-yl]methyl}-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[(pyridin-4-yl)methyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1R)-1-(3-cyanopyridin-2-yl)ethyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1S)-1-(1-methyl-1H-benzimidazol-2-yl)ethyl]-5′,6′-dihydrospiro[pyrrolidine-3,4′-pyrrolo[1,2-b]pyrazole]-1-carboxamide (diastereomeric mixture)
2′-[6-amino-5-(trifluoromethyl)pyridin-3-yl]-N-[1-(4-fluorophenyl)-2-methoxyethyl]-5′,6′-dihydrospiro[pyrrolidine-3,4′-pyrrolo[1,2-b]pyrazole]-1-carboxamide (mixture of isomers)
(rac)-2′-[6-amino-5-(trifluoromethyl)pyridin-3-yl]-N-[(pyridin-2-yl)methyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1S)-1-(1-ethyl-1H-imidazol-2-yl)ethyl]-5′,6′-dihydrospiro[pyrrolidine-3,4′-pyrrolo[1,2-b]pyrazole]-1-carboxamide (diastereomeric mixture)
2′-[6-amino-5-(trifluoromethyl)pyridin-3-yl]-N-[(1S)-1-(1-methyl-1H-imidazol-2-yl)ethyl]-5′,6′-dihydrospiro[pyrrolidine-3,4′-pyrrolo[1,2-b]pyrazole]-1-carboxamide (diastereomeric mixture)
(rac)-2′-[6-amino-5-(trifluoromethyl)pyridin-3-yl]-N-[(pyrazin-2-yl)methyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1R)-2-methoxy-1-(pyridin-3-yl)ethyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[(pyridazin-4-yl)methyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[(1-methylpiperidin-4-yl)methyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[3-(methylsulfamoyl)pyridin-2-yl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[(1-methyl-2-oxabicyclo[2.1.1]hexan-4-yl)methyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[(1-methyl-2-oxopiperidin-4-yl)methyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-(2-cyclopropylpropan-2-yl)-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1R)-1-(4-cyanophenyl)ethyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[1-methyl-2,3-dihydro-1H-inden-1-yl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[cyclopropyl(phenyl)methyl]-5′,6′-dihydrospiro[pyrrolidine-3,4′-pyrrolo[1,2-b]pyrazole]-1-carboxamide (mixture of isomers)
2′-[6-amino-5-(trifluoromethyl)pyridin-3-yl]-N-[−2-methyl-1-phenylpropyl]-5′,6′-dihydrospiro[pyrrolidine-3,4′-pyrrolo[1,2-b]pyrazole]-1-carboxamide (mixture of isomers)
2′-[6-amino-5-(trifluoromethyl)pyridin-3-yl]-N-[(1S)-1-(pyridin-4-yl)ethyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[2,2,2-trifluoro-1-phenylethyl]-5′,6′-dihydrospiro[pyrrolidine-3,4′-pyrrolo[1,2-b]pyrazole]-1-carboxamide (mixture of isomers)
2′-[6-amino-5-(trifluoromethyl)pyridin-3-yl]-N-[1-(pyrazin-2-yl)ethyl]-5′,6′-dihydrospiro[pyrrolidine-3,4′-pyrrolo[1,2-b]pyrazole]-1-carboxamide (mixture of isomers)
2′-[6-amino-5-(trifluoromethyl)pyridin-3-yl]-N-[(1R)-2-methoxy-1-phenylethyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[(pyridazin-3-yl)methyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[1-(2-cyanophenyl)cyclobutyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1S)-2,3-dihydro-1H-inden-1-yl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1S)-1-(4-fluorophenyl)ethyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1R)-1-(2-cyanophenyl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(1R)-1-(6-cyanopyridin-3-yl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[1-(pyridin-2-yl)cyclobutyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(pyridin-4-yl)methyl]-5′,6′-dihydrospiro[azetidine-3,4′-pyrrolo[1,2-b]pyrazole]-1-carboxamide
(3′RS)-2-[6-amino-5-(trifluoromethyl)pyridin-3-yl]-N-[(1R)-1-(4-cyanophenyl)ethyl]-6,7-dihydrospiro[pyrazolo[5,1-c][1,4]oxazine-4,3′-pyrrolidine]-1′-carboxamide
2′-[6-amino-5-(trifluoromethyl)pyridin-3-yl]-N-[(1R)-1-(3-cyanopyridin-2-yl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(1R)-1-(4-cyanophenyl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(1R)-1-(2-cyanopyridin-4-yl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(1R)-1-(2-chloro-4-cyanophenyl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[2-(2-fluorophenyl)propan-2-yl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[1-(1-ethyl-3,5-dimethyl-1H-pyrazol-4-yl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[2-(pyridin-2-yl)propan-2-yl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[1-(1-methyl-1H-pyrazol-4-yl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[1-(1,3,4-trimethyl-1H-pyrazol-5-yl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-{1-[3-(difluoromethyl)-1-methyl-1H-pyrazol-4-yl]ethyl}-5′,6′-dihydrospiro[azetidine-3,4′-pyrrolo[1,2-b]pyrazole]-1-carboxamide
(rac)-2-[6-amino-5-(trifluoromethyl)pyridin-3-yl]-N-[2-(pyridin-4-yl)propan-2-yl]-6,7-dihydrospiro[pyrazolo[5,1-c][1,4]oxazine-4,3′-pyrrolidine]-1′-carboxamide
(rac)-2-[6-amino-5-(trifluoromethyl)pyridin-3-yl]-N-[1-(pyridin-3-yl)cyclobutyl]-6,7-dihydrospiro[pyrazolo[5,1-c][1,4]oxazine-4,3′-pyrrolidine]-1′-carboxamide
2′-[6-amino-5-(trifluoromethyl)pyridin-3-yl]-N-[1-(1-ethyl-5-methyl-1H-pyrazol-4-yl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[1-(1,5-dimethyl-1H-pyrazol-4-yl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[1-(1,3,5-trimethyl-1H-pyrazol-4-yl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(1R)-1-(pyridin-3-yl)ethyl]-5′,6′-dihydrospiro[azetidine-3,4′-pyrrolo[1,2-b]pyrazole]-1-carboxamide
2-[6-amino-5-(trifluoromethyl)pyridin-3-yl]-N-[(1R)-1-(2-cyanopyridin-4-yl)ethyl]-6,7-dihydrospiro[pyrazolo[5,1-c][1,4]oxazine-4,3′-pyrrolidine]-1′-carboxamide
2-[6-amino-5-(trifluoromethyl)pyridin-3-yl]-N-[(1R)-1-(3-cyanopyridin-2-yl)ethyl]-6,7-dihydrospiro[pyrazolo[5,1-c][1,4]oxazine-4,3′-pyrrolidine]-1′-carboxamide
2-[6-amino-5-(trifluoromethyl)pyridin-3-yl]-N-[(1R)-1-(6-cyanopyridin-3-yl)ethyl]-6,7-dihydrospiro[pyrazolo[5,1-c][1,4]oxazine-4,3′-pyrrolidine]-1′-carboxamide
2′-[6-amino-5-(trifluoromethyl)pyridin-3-yl]-N-[(pyridin-2-yl)methyl]-5′,6′-dihydrospiro[azetidine-3,4′-pyrrolo[1,2-b]pyrazole]-1-carboxamide
(rac)-2-[6-amino-5-(trifluoromethyl)pyridin-3-yl]-N-[(1R)-1-(2-cyanophenyl)ethyl]-6,7-dihydrospiro[pyrazolo[5,1-c][1,4]oxazine-4,3′-pyrrolidine]-1′-carboxamide
2′-[6-amino-5-(trifluoromethyl)pyridin-3-yl]-N-[(1R)-1-(4-fluorophenyl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[2-(4-fluorophenyl)propan-2-yl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[2-(pyridin-3-yl)propan-2-yl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(2-fluorophenyl)methyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[1-(pyridin-3-yl)cyclobutyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(4-fluorophenyl)methyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[2-(pyridin-4-yl)propan-2-yl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[2-(3-fluorophenyl)propan-2-yl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(1S)-1-(3-chloropyridin-4-yl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(1-methyl-2-oxabicyclo[2.1.1]hexan-4-yl)methyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-{[3-(trifluoromethyl)bicyclo[1.1.1]pentan-1-yl]methyl}-5′,6′-dihydrospiro[azetidine-3,4′-pyrrolo[1,2-b]pyrazole]-1-carboxamide
2′-[6-amino-5-(trifluoromethyl)pyridin-3-yl]-N-[2-(2-chlorophenyl)propan-2-yl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(1R)-1-(3-fluorophenyl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[2-(3-fluoropyridin-2-yl)propan-2-yl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[1-(2-chlorophenyl)cyclobutyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[1-(3-fluorophenyl)cyclobutyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(1R)-1-phenylpropyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(pyrimidin-4-yl)methyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(3-fluoropyridin-2-yl)methyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(pyrimidin-5-yl)methyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(3-chloropyridin-4-yl)methyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(3-fluorophenyl)methyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(pyridin-3-yl)methyl]-5′,6′-dihydrospiro[azetidine-3,4′-pyrrolo[1,2-b]pyrazole]-1-carboxamide
(rac)-2-[6-amino-5-(trifluoromethyl)pyridin-3-yl]-N-benzyl-6,7-dihydrospiro[pyrazolo[5,1-c][1,4]oxazine-4,3′-pyrrolidine]-1′-carboxamide
(rac)-2-[6-amino-5-(trifluoromethyl)pyridin-3-yl]-N-[(1R)-1-(2-methylpyrimidin-5-yl)ethyl]-6,7-dihydrospiro[pyrazolo[5,1-c][1,4]oxazine-4,3′-pyrrolidine]-1′-carboxamide
(rac)-2-[6-amino-5-(trifluoromethyl)pyridin-3-yl]-N-[(1R)-1-(pyridin-3-yl)ethyl]-6,7-dihydrospiro[pyrazolo[5,1-c][1,4]oxazine-4,3′-pyrrolidine]-1′-carboxamide
2′-[6-amino-5-(trifluoromethyl)pyridin-3-yl]-N-benzyl-5′,6′-dihydrospiro[azetidine-3,4′-pyrrolo[1,2-b]pyrazole]-1-carboxamide
2-[6-amino-5-(trifluoromethyl)pyridin-3-yl]-N-[2-(2-fluorophenyl)propan-2-yl]-6,7-dihydrospiro[pyrazolo[5,1-c][1,4]oxazine-4,3′-pyrrolidine]-1′-carboxamide (enantiomer 1)
2-[6-amino-5-(trifluoromethyl)pyridin-3-yl]-N-[2-(3-chloropyridin-4-yl)propan-2-yl]-6,7-dihydrospiro[pyrazolo[5,1-c][1,4]oxazine-4,3′-pyrrolidine]-1′-carboxamide (enantiomer 1)
2-[6-amino-5-(trifluoromethyl)pyridin-3-yl]-N-[2-(2-fluorophenyl)propan-2-yl]-6,7-dihydrospiro[pyrazolo[5,1-c][1,4]oxazine-4,3′-pyrrolidine]-1′-carboxamide (enantiomer 2)
2-[6-amino-5-(trifluoromethyl)pyridin-3-yl]-N-[2-(3-chloropyridin-4-yl)propan-2-yl]-6,7-dihydrospiro[pyrazolo[5,1-c][1,4]oxazine-4,3′-pyrrolidine]-1′-carboxamide (enantiomer 2)
2-[6-amino-5-(trifluoromethyl)pyridin-3-yl]-N-[(1R)-1-(4-cyanopyridin-2-yl)ethyl]-6,7-dihydrospiro[pyrazolo[5,1-c][1,4]oxazine-4,3′-pyrrolidine]-1′-carboxamide
(rac)-2-[6-amino-5-(trifluoromethyl)pyridin-3-yl]-N-[2-(1-methyl-1H-1,2,3-triazol-5-yl)propan-2-yl]-6,7-dihydrospiro[pyrazolo[5,1-c][1,4]oxazine-4,3′-pyrrolidine]-1′-carboxamide
(rac)-2-[6-amino-5-(trifluoromethyl)pyridin-3-yl]-N-[2-(pyridin-3-yl)propan-2-yl]-6,7-dihydrospiro[pyrazolo[5,1-c][1,4]oxazine-4,3′-pyrrolidine]-1′-carboxamide
2-[6-amino-5-(trifluoromethyl)pyridin-3-yl]-N-[(1R)-1-(4-fluorophenyl)ethyl]-6,7-dihydrospiro[pyrazolo[5,1-c][1,4]oxazine-4,3′-pyrrolidine]-1′-carboxamide
2-[6-amino-5-(trifluoromethyl)pyridin-3-yl]-N-[(1R)-2,3-dihydro-1H-inden-1-yl]-6,7-dihydrospiro[pyrazolo[5,1-c][1,4]oxazine-4,3′-pyrrolidine]-1′-carboxamide
2-[6-amino-5-(trifluoromethyl)pyridin-3-yl]-N-[(1R)-1-(5-fluoropyridin-3-yl)ethyl]-6,7-dihydrospiro[pyrazolo[5,1-c][1,4]oxazine-4,3′-pyrrolidine]-1′-carboxamide
2-[6-amino-5-(trifluoromethyl)pyridin-3-yl]-N-[(1R)-1-(3-fluorophenyl)ethyl]-6,7-dihydrospiro[pyrazolo[5,1-c][1,4]oxazine-4,3′-pyrrolidine]-1′-carboxamide
2-[6-amino-5-(trifluoromethyl)pyridin-3-yl]-N-[(1R)-1-phenylpropyl]-6,7-dihydrospiro[pyrazolo[5,1-c][1,4]oxazine-4,3′-pyrrolidine]-1′-carboxamide
(3′RS)-2-[6-amino-5-(trifluoromethyl)pyridin-3-yl]-N-[(3S)-2,3-dihydro-1-benzofuran-3-yl]-6,7-dihydrospiro[pyrazolo[5,1-c][1,4]oxazine-4,3′-pyrrolidine]-1′-carboxamide
(rac)-2-[6-amino-5-(trifluoromethyl)pyridin-3-yl]-N-[(bicyclo[1.1.1]pentan-1-yl)methyl]-6,7-dihydrospiro[pyrazolo[5,1-c][1,4]oxazine-4,3′-pyrrolidine]-1′-carboxamide
2-[6-amino-5-(trifluoromethyl)pyridin-3-yl]-N-[(1S)-2-methoxy-1-phenylethyl]-6,7-dihydrospiro[pyrazolo[5,1-c][1,4]oxazine-4,3′-pyrrolidine]-1′-carboxamide
2′-[6-amino-5-(trifluoromethyl)pyridin-3-yl]-N-[(1R)-1-(1,2-oxazol-3-yl)ethyl]-5′,6′-dihydrospiro[azetidine-3,4′-pyrrolo[1,2-b]pyrazole]-1-carboxamide
2-[6-amino-5-(trifluoromethyl)pyridin-3-yl]-N-[(1R)-1-(2-fluorophenyl)ethyl]-6,7-dihydrospiro[pyrazolo[5,1-c][1,4]oxazine-4,3′-pyrrolidine]-1′-carboxamide
(rac)-2-[6-amino-5-(trifluoromethyl)pyridin-3-yl]-N-[2-(3-fluorophenyl)propan-2-yl]-6,7-dihydrospiro[pyrazolo[5,1-c][1,4]oxazine-4,3′-pyrrolidine]-1′-carboxamide
2′-[6-amino-5-(trifluoromethyl)pyridin-3-yl]-N-[(1R)-1-(1-phenyl-1H-pyrazol-4-yl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(1S)-2-methoxy-1-phenylethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(3-fluorobicyclo[1.1.1]pentan-1-yl)methyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(bicyclo[1.1.1]pentan-1-yl)methyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(1S)-1-(pyridin-3-yl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(1R)-1-(2-methylpyrimidin-5-yl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[2-(1-methyl-1H-1,2,3-triazol-4-yl)propan-2-yl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[2-(3-chloropyridin-4-yl)propan-2-yl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[1-(pyridin-4-yl)cyclobutyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(1R)-1-phenylethyl]-5′,6′-dihydrospiro[azetidine-3,4′-pyrrolo[1,2-b]pyrazole]-1-carboxamide
2-[6-amino-5-(trifluoromethyl)pyridin-3-yl]-N-[(1S)-1-(pyridin-3-yl)ethyl]-6,7-dihydrospiro[pyrazolo[5,1-c][1,4]oxazine-4,3′-pyrrolidine]-1′-carboxamide
(rac)-2-[6-amino-5-(trifluoromethyl)pyridin-3-yl]-N-[(3-fluorobicyclo[1.1.1]pentan-1-yl)methyl]-6,7-dihydrospiro[pyrazolo[5,1-c][1,4]oxazine-4,3′-pyrrolidine]-1′-carboxamide
2-[6-amino-5-(trifluoromethyl)pyridin-3-yl]-N-[(1S)-1-(pyridin-4-yl)ethyl]-6,7-dihydrospiro[pyrazolo[5,1-c][1,4]oxazine-4,3′-pyrrolidine]-1′-carboxamide
2′-[6-amino-5-(trifluoromethyl)pyridin-3-yl]-N-(1-phenylcyclobutyl)-5′,6′-dihydrospiro[azetidine-3,4′-pyrrolo[1,2-b]pyrazole]-1-carboxamide
2′-(6-amino-5-cyanopyridin-3-yl)-N-[1-(pyridin-4-yl)cyclobutyl]-5′,6′-dihydrospiro[azetidine-3,4′-pyrrolo[1,2-b]pyrazole]-1-carboxamide
2′-(6-amino-5-cyanopyridin-3-yl)-N-[1-(3-fluorophenyl)cyclobutyl]-5′,6′-dihydrospiro[azetidine-3,4′-pyrrolo[1,2-b]pyrazole]-1-carboxamide
2′-(6-amino-5-cyanopyridin-3-yl)-N-[2-(3-chloropyridin-4-yl)propan-2-yl]-5′,6′-dihydrospiro[azetidine-3,4′-pyrrolo[1,2-b]pyrazole]-1-carboxamide
2′-(6-amino-5-cyanopyridin-3-yl)-N-[(1R)-1-(5-fluoropyridin-3-yl)ethyl]-5′,6′-dihydrospiro[azetidine-3,4′-pyrrolo[1,2-b]pyrazole]-1-carboxamide
2′-(6-amino-5-cyanopyridin-3-yl)-N-[(1R)-1-(4-fluorophenyl)ethyl]-5′,6′-dihydrospiro[azetidine-3,4′-pyrrolo[1,2-b]pyrazole]-1-carboxamide
2′-(6-amino-5-cyanopyridin-3-yl)-N-[2-(3-fluorophenyl)propan-2-yl]-5′,6′-dihydrospiro[azetidine-3,4′-pyrrolo[1,2-b]pyrazole]-1-carboxamide
2′-(6-amino-5-cyanopyridin-3-yl)-N-[(1S)-1-(3-chloropyridin-4-yl)ethyl]-5′,6′-dihydrospiro[azetidine-3,4′-pyrrolo[1,2-b]pyrazole]-1-carboxamide
2′-(6-amino-5-cyanopyridin-3-yl)-N-[(1-methyl-2-oxabicyclo[2.1.1]hexan-4-yl)methyl]-5′,6′-dihydrospiro[azetidine-3,4′-pyrrolo[1,2-b]pyrazole]-1-carboxamide
2′-(6-amino-5-cyanopyridin-3-yl)-N-[(1R)-1-(4-cyanopyridin-2-yl)ethyl]-5′,6′-dihydrospiro[azetidine-3,4′-pyrrolo[1,2-b]pyrazole]-1-carboxamide
2′-(6-amino-5-cyanopyridin-3-yl)-N-[(1R)-1-(2-cyanopyridin-4-yl)ethyl]-5′,6′-dihydrospiro[azetidine-3,4′-pyrrolo[1,2-b]pyrazole]-1-carboxamide
2′-(6-amino-5-cyanopyridin-3-yl)-N-[(1R)-1-(2-chloro-4-cyanophenyl)ethyl]-5′,6′-dihydrospiro[azetidine-3,4′-pyrrolo[1,2-b]pyrazole]-1-carboxamide
2′-(6-amino-5-cyanopyridin-3-yl)-N-[1-(2-fluorophenyl)cyclobutyl]-5′,6′-dihydrospiro[azetidine-3,4′-pyrrolo[1,2-b]pyrazole]-1-carboxamide
2′-(6-amino-5-cyanopyridin-3-yl)-N-[2-(4-fluorophenyl)propan-2-yl]-5′,6′-dihydrospiro[azetidine-3,4′-pyrrolo[1,2-b]pyrazole]-1-carboxamide
2′-(6-amino-5-cyanopyridin-3-yl)-N-[1-(2-chlorophenyl)cyclobutyl]-5′,6′-dihydrospiro[azetidine-3,4′-pyrrolo[1,2-b]pyrazole]-1-carboxamide
2′-(6-amino-5-cyanopyridin-3-yl)-N-[2-(1-methyl-1H-1,2,3-triazol-4-yl)propan-2-yl]-5′,6′-dihydrospiro[azetidine-3,4′-pyrrolo[1,2-b]pyrazole]-1-carboxamide
2′-(6-amino-5-cyanopyridin-3-yl)-N-[2-(pyridin-2-yl)propan-2-yl]-5′,6′-dihydrospiro[azetidine-3,4′-pyrrolo[1,2-b]pyrazole]-1-carboxamide
2′-(6-amino-5-cyanopyridin-3-yl)-N-[2-(pyridin-4-yl)propan-2-yl]-5′,6′-dihydrospiro[azetidine-3,4′-pyrrolo[1,2-b]pyrazole]-1-carboxamide
2′-(6-amino-5-cyanopyridin-3-yl)-N-[(1R)-1-phenylpropyl]-5′,6′-dihydrospiro[azetidine-3,4′-pyrrolo[1,2-b]pyrazole]-1-carboxamide
2′-(6-amino-5-cyanopyridin-3-yl)-N-[(1R)-1-(3-fluorophenyl)ethyl]-5′,6′-dihydrospiro[azetidine-3,4′-pyrrolo[1,2-b]pyrazole]-1-carboxamide
2′-(6-amino-5-cyanopyridin-3-yl)-N-[(1R)-1-(2-cyanophenyl)ethyl]-5′,6′-dihydrospiro[azetidine-3,4′-pyrrolo[1,2-b]pyrazole]-1-carboxamide
(rac)-2′-(6-amino-5-cyanopyridin-3-yl)-N-[2-(pyridin-4-yl)propan-2-yl]-5′,6′-dihydrospiro[pyrrolidine-3,4′-pyrrolo[1,2-b]pyrazole]-1-carboxamide
2′-(6-amino-5-cyanopyridin-3-yl)-N-[(1R)-1-(5-fluoropyridin-3-yl)ethyl]-5′,6′-dihydrospiro[pyrrolidine-3,4′-pyrrolo[1,2-b]pyrazole]-1-carboxamide
(rac)-2′-(6-amino-5-cyanopyridin-3-yl)-N-[2-(1-methyl-1H-1,2,3-triazol-4-yl)propan-2-yl]-5′,6′-dihydrospiro[pyrrolidine-3,4′-pyrrolo[1,2-b]pyrazole]-1-carboxamide
(rac)-2-(6-amino-5-cyanopyridin-3-yl)-N-[1-(pyridin-2-yl)cyclobutyl]-6,7-dihydrospiro[pyrazolo[5,1-c][1,4]oxazine-4,3′-pyrrolidine]-1′-carboxamide
(rac)-2′-(6-amino-5-cyanopyridin-3-yl)-N-[1-(pyridin-2-yl)cyclobutyl]-5′,6′-dihydrospiro[pyrrolidine-3,4′-pyrrolo[1,2-b]pyrazole]-1-carboxamide
(rac)-2′-(6-amino-5-cyanopyridin-3-yl)-N-[1-(2-chlorophenyl)cyclobutyl]-5′,6′-dihydrospiro[pyrrolidine-3,4′-pyrrolo[1,2-b]pyrazole]-1-carboxamide
2-(6-amino-5-cyanopyridin-3-yl)-N-[(1R)-1-phenylethyl]-6,7-dihydrospiro[pyrazolo[5,1-c][1,4]oxazine-4,3′-pyrrolidine]-1′-carboxamide
2′-(6-amino-5-cyanopyridin-3-yl)-N-[(1R)-1-phenylethyl]-5′,6′-dihydrospiro[pyrrolidine-3,4′-pyrrolo[1,2-b]pyrazole]-1-carboxamide
(rac)-2-(6-amino-5-cyanopyridin-3-yl)-N-[1-(2-chlorophenyl)cyclobutyl]-6,7-dihydrospiro[pyrazolo[5,1-c][1,4]oxazine-4,3′-pyrrolidine]-1′-carboxamide
(rac)-2-(6-amino-5-cyanopyridin-3-yl)-N-[1-(4-fluorophenyl)cyclobutyl]-6,7-dihydrospiro[pyrazolo[5,1-c][1,4]oxazine-4,3′-pyrrolidine]-1′-carboxamide
(rac)-2-(6-amino-5-cyanopyridin-3-yl)-N-[2-(4-fluorophenyl)propan-2-yl]-6,7-dihydrospiro[pyrazolo[5,1-c][1,4]oxazine-4,3′-pyrrolidine]-1′-carboxamide
(rac)-2-(6-amino-5-cyanopyridin-3-yl)-N-[2-(pyridin-2-yl)propan-2-yl]-6,7-dihydrospiro[pyrazolo[5,1-c][1,4]oxazine-4,3′-pyrrolidine]-1′-carboxamide
(rac)-2′-(6-amino-5-cyanopyridin-3-yl)-N-[2-(pyridin-2-yl)propan-2-yl]-5′,6′-dihydrospiro[pyrrolidine-3,4′-pyrrolo[1,2-b]pyrazole]-1-carboxamide
2-(6-amino-5-cyanopyridin-3-yl)-N-[1-(pyridin-4-yl)cyclobutyl]-6,7-dihydrospiro[pyrazolo[5,1-c][1,4]oxazine-4,3′-pyrrolidine]-1′-carboxamide (enantiomer 2)
2-(6-amino-5-cyanopyridin-3-yl)-N-[1-(2-chlorophenyl)cyclobutyl]-6,7-dihydrospiro[pyrazolo[5,1-c][1,4]oxazine-4,3′-pyrrolidine]-1′-carboxamide (enantiomer 2)
2-(6-amino-5-cyanopyridin-3-yl)-N-[2-(pyridin-2-yl)propan-2-yl]-6,7-dihydrospiro[pyrazolo[5,1-c][1,4]oxazine-4,3′-pyrrolidine]-1′-carboxamide (enantiomer 1)
2-(6-amino-5-cyanopyridin-3-yl)-N-[1-(2-chlorophenyl)cyclobutyl]-6,7-dihydrospiro[pyrazolo[5,1-c][1,4]oxazine-4,3′-pyrrolidine]-1′-carboxamide (enantiomer 1)
2′-(6-amino-5-cyanopyridin-3-yl)-N-[1-(pyridin-3-yl)cyclobutyl]-5′,6′-dihydrospiro[azetidine-3,4′-pyrrolo[1,2-b]pyrazole]-1-carboxamide
2′-(6-amino-5-cyanopyridin-3-yl)-N-[1-(pyridin-2-yl)cyclobutyl]-5′,6′-dihydrospiro[azetidine-3,4′-pyrrolo[1,2-b]pyrazole]-1-carboxamide
2-(6-amino-5-cyanopyridin-3-yl)-N-[1-(pyridin-4-yl)cyclobutyl]-6,7-dihydrospiro[pyrazolo[5,1-c][1,4]oxazine-4,3′-pyrrolidine]-1′-carboxamide (enantiomer 1)
2′-(6-amino-5-cyanopyridin-3-yl)-N-(1-phenylcyclobutyl)-5′,6′-dihydrospiro[azetidine-3,4′-pyrrolo[1,2-b]pyrazole]-1-carboxamide
2′-(6-amino-5-cyanopyridin-3-yl)-N-[(3S)-2,3-dihydro-1-benzofuran-3-yl]-5′,6′-dihydrospiro[pyrrolidine-3,4′-pyrrolo[1,2-b]pyrazole]-1-carboxamide
2-(6-amino-5-cyanopyridin-3-yl)-N-[(3S)-2,3-dihydro-1-benzofuran-3-yl]-6,7-dihydrospiro[pyrazolo[5,1-c][1,4]oxazine-4,3′-pyrrolidine]-1′-carboxamide
2-(6-amino-5-cyanopyridin-3-yl)-N-[(1R)-1-(2-fluorophenyl)ethyl]-6,7-dihydrospiro[pyrazolo[5,1-c][1,4]oxazine-4,3′-pyrrolidine]-1′-carboxamide
2′-(6-amino-5-cyanopyridin-3-yl)-N-[(1R)-1-(2-fluorophenyl)ethyl]-5′,6′-dihydrospiro[pyrrolidine-3,4′-pyrrolo[1,2-b]pyrazole]-1-carboxamide
(rac)-2′-(6-amino-5-cyanopyridin-3-yl)-N-[2-(4-fluorophenyl)propan-2-yl]-5′,6′-dihydrospiro[pyrrolidine-3,4′-pyrrolo[1,2-b]pyrazole]-1-carboxamide
(rac)-2-(6-amino-5-cyanopyridin-3-yl)-N-[2-(1-methyl-1H-1,2,3-triazol-4-yl)propan-2-yl]-6,7-dihydrospiro[pyrazolo[5,1-c][1,4]oxazine-4,3′-pyrrolidine]-1′-carboxamide
(rac)-2-(6-amino-5-cyanopyridin-3-yl)-N-[(1R)-1-(5-fluoropyridin-3-yl)ethyl]-6,7-dihydrospiro[pyrazolo[5,1-c][1,4]oxazine-4,3′-pyrrolidine]-1′-carboxamide
(rac)-2′-(6-amino-5-cyanopyridin-3-yl)-N-(1-phenylcyclobutyl)-5′,6′-dihydrospiro[pyrrolidine-3,4′-pyrrolo[1,2-b]pyrazole]-1-carboxamide
(rac)-2′-(6-amino-5-cyanopyridin-3-yl)-N-[1-(4-fluorophenyl)cyclobutyl]-5′,6′-dihydrospiro[pyrrolidine-3,4′-pyrrolo[1,2-b]pyrazole]-1-carboxamide
(rac)-2-(6-amino-5-cyanopyridin-3-yl)-N-[1-(pyridin-4-yl)cyclobutyl]-6,7-dihydrospiro[pyrazolo[5,1-c][1,4]oxazine-4,3′-pyrrolidine]-1′-carboxamide
(rac)-2′-(6-amino-5-cyanopyridin-3-yl)-N-[1-(pyridin-4-yl)cyclobutyl]-5′,6′-dihydrospiro[pyrrolidine-3,4′-pyrrolo[1,2-b]pyrazole]-1-carboxamide
(rac)-2-(6-amino-5-cyanopyridin-3-yl)-N-(1-phenylcyclobutyl)-6,7-dihydrospiro[pyrazolo[5,1-c][1,4]oxazine-4,3′-pyrrolidine]-1′-carboxamide
2′-(6-amino-5-cyanopyridin-3-yl)-N-[(1R)-1-(1,3,4-trimethyl-1H-pyrazol-5-yl)ethyl]-5′,6′-dihydrospiro[azetidine-3,4′-pyrrolo[1,2-b]pyrazole]-1-carboxamide
(rac)-2-(6-amino-5-cyanopyridin-3-yl)-N-[2-(pyridin-4-yl)propan-2-yl]-6,7-dihydrospiro[pyrazolo[5,1-c][1,4]oxazine-4,3′-pyrrolidine]-1′-carboxamide
2′-[6-amino-5-(difluoromethoxy)pyridin-3-yl]-N-[1-(pyridin-3-yl)cyclobutyl]-5′,6′-dihydrospiro[azetidine-3,4′-pyrrolo[1,2-b]pyrazole]-1-carboxamide
(rac)-2′-[6-amino-5-(trifluoromethoxy)pyridin-3-yl]-N-[2-(1-methyl-1H-1,2,3-triazol-4-yl)propan-2-yl]5′,6′-dihydrospiro[pyrrolidine-3,4′-pyrrolo[1,2-b]pyrazole]-1-carboxamide
2′-[6-amino-5-(trifluoromethoxy)pyridin-3-yl]-N-[(1R)-1-(2-methylpyrimidin-5-yl)ethyl]-5′,6′-dihydrospiro[pyrrolidine-3,4′-pyrrolo[1,2-b]pyrazole]-1-carboxamide
2′-[6-amino-5-(trifluoromethoxy)pyridin-3-yl]-N-[(1R)-1-(pyridin-2-yl)ethyl]-5′,6′-dihydrospiro[pyrrolidine-3,4′-pyrrolo[1,2-b]pyrazole]-1-carboxamide
2′-[6-amino-5-(trifluoromethoxy)pyridin-3-yl]-N-[(1R)-1-phenylethyl]-5′,6′-dihydrospiro[pyrrolidine-3,4′-pyrrolo[1,2-b]pyrazole]-1-carboxamide (mixture of stereoisomer)
2′-[6-amino-5-(trifluoromethoxy)pyridin-3-yl]-N-[(1R)-1-(pyridin-3-yl)ethyl]-5′,6′-dihydrospiro[pyrrolidine-3,4′-pyrrolo[1,2-b]pyrazole]-1-carboxamide
2′-[6-amino-5-(trifluoromethoxy)pyridin-3-yl]-N-[(1S)-1-(pyridin-4-yl)ethyl]-5′,6′-dihydrospiro[pyrrolidine-3,4′-pyrrolo[1,2-b]pyrazole]-1-carboxamide
(rac)-2′-[6-amino-5-(trifluoromethoxy)pyridin-3-yl]-N-[2-(4-fluorophenyl)propan-2-yl]-5′,6′-dihydrospiro[pyrrolidine-3,4′-pyrrolo[1,2-b]pyrazole]-1-carboxamide
2′-[6-amino-5-(trifluoromethoxy)pyridin-3-yl]-N-[(1R)-1-(4-fluorophenyl)ethyl]-5′,6′-dihydrospiro[pyrrolidine-3,4′-pyrrolo[1,2-b]pyrazole]-1-carboxamide
(rac)-2-[6-amino-5-(trifluoromethoxy)pyridin-3-yl]-N-[2-(pyridin-4-yl)propan-2-yl]-6,7-dihydrospiro[pyrazolo[5,1-c][1,4]oxazine-4,3′-pyrrolidine]-1′-carboxamide
(rac)-2-[6-amino-5-(trifluoromethoxy)pyridin-3-yl]-N-[1-(pyridin-4-yl)cyclobutyl]-6,7-dihydrospiro[pyrazolo[5,1-c][1,4]oxazine-4,3′-pyrrolidine]-1′-carboxamide
2′-[6-amino-5-(difluoromethoxy)pyridin-3-yl]-N-(1-phenylcyclobutyl)-5′,6′-dihydrospiro[azetidine-3,4′-pyrrolo[1,2-b]pyrazole]-1-carboxamide
(rac)-2′-[6-amino-5-(trifluoromethoxy)pyridin-3-yl]-N-[2-(pyridin-2-yl)propan-2-yl]-5′,6′-dihydrospiro[pyrrolidine-3,4′-pyrrolo[1,2-b]pyrazole]-1-carboxamide
(rac)-2′-[6-amino-5-(trifluoromethoxy)pyridin-3-yl]-N-[2-(3-chloropyridin-4-yl)propan-2-yl]-5′,6′-dihydrospiro[pyrrolidine-3,4′-pyrrolo[1,2-b]pyrazole]-1-carboxamide
2′-[6-amino-5-(difluoromethoxy)pyridin-3-yl]-N-[2-(2-fluorophenyl)propan-2-yl]-5′,6′-dihydrospiro[azetidine-3,4′-pyrrolo[1,2-b]pyrazole]-1-carboxamide
(rac)-2-[6-amino-5-(difluoromethoxy)pyridin-3-yl]-N-[2-(3-fluorophenyl)propan-2-yl]-6,7-dihydrospiro[pyrazolo[5,1-c][1,4]oxazine-4,3′-pyrrolidine]-1′-carboxamide
2-[6-amino-5-(difluoromethoxy)pyridin-3-yl]-N-[(1S)-1-(3-chloropyridin-4-yl)ethyl]-6,7-dihydrospiro[pyrazolo[5,1-c][1,4]oxazine-4,3′-pyrrolidine]-1′-carboxamide
2-[6-amino-5-(trifluoromethoxy)pyridin-3-yl]-N-[1-(1,3,5-trimethyl-1H-pyrazol-4-yl)ethyl]-6,7-dihydrospiro[pyrazolo[5,1-c][1,4]oxazine-4,3′-pyrrolidine]-1′-carboxamide
2-[6-amino-5-(trifluoromethoxy)pyridin-3-yl]-N-[1-(1-ethyl-3,5-dimethyl-1H-pyrazol-4-yl)ethyl]-6,7-dihydrospiro[pyrazolo[5,1-c][1,4]oxazine-4,3′-pyrrolidine]-1′-carboxamide
2-[6-amino-5-(trifluoromethoxy)pyridin-3-yl]-N-[(1R)-1-(2-cyanophenyl)ethyl]-6,7-dihydrospiro[pyrazolo[5,1-c][1,4]oxazine-4,3′-pyrrolidine]-1′-carboxamide
(rac)-2′-[6-amino-5-(difluoromethoxy)pyridin-3-yl]-N-[2-(pyridin-2-yl)propan-2-yl]-5′,6′-dihydrospiro[pyrrolidine-3,4′-pyrrolo[1,2-b]pyrazole]-1-carboxamide
2-[6-amino-5-(trifluoromethoxy)pyridin-3-yl]-N-[(1R)-1-(4-fluorophenyl)ethyl]-6,7-dihydrospiro[pyrazolo[5,1-c][1,4]oxazine-4,3′-pyrrolidine]-1′-carboxamide
2-[6-amino-5-(trifluoromethoxy)pyridin-3-yl]-N-[(1R)-1-phenylpropyl]-6,7-dihydrospiro[pyrazolo[5,1-c][1,4]oxazine-4,3′-pyrrolidine]-1′-carboxamide
2-[6-amino-5-(trifluoromethoxy)pyridin-3-yl]-N-[(1R)-1-phenylethyl]-6,7-dihydrospiro[pyrazolo[5,1-c][1,4]oxazine-4,3′-pyrrolidine]-1′-carboxamide
(rac)-2-[6-amino-5-(trifluoromethoxy)pyridin-3-yl]-N-[1-(pyridin-3-yl)cyclobutyl]-6,7-dihydrospiro[pyrazolo[5,1-c][1,4]oxazine-4,3′-pyrrolidine]-1′-carboxamide
2-[6-amino-5-(difluoromethoxy)pyridin-3-yl]-N-[(1R)-1-(2-chloro-4-cyanophenyl)ethyl]-6,7-dihydrospiro[pyrazolo[5,1-c][1,4]oxazine-4,3′-pyrrolidine]-1′-carboxamide
(rac)-2-[6-amino-5-(difluoromethoxy)pyridin-3-yl]-N-[2-(4-fluorophenyl)propan-2-yl]-6,7-dihydrospiro[pyrazolo[5,1-c][1,4]oxazine-4,3′-pyrrolidine]-1′-carboxamide
(rac)-2-[6-amino-5-(difluoromethoxy)pyridin-3-yl]-N-[1-(2-fluorophenyl)cyclobutyl]-6,7-dihydrospiro[pyrazolo[5,1-c][1,4]oxazine-4,3′-pyrrolidine]-1′-carboxamide
(rac)-2′-[6-amino-5-(difluoromethoxy)pyridin-3-yl]-N-[2-(pyridin-3-yl)propan-2-yl]-5′,6′-dihydrospiro[pyrrolidine-3,4′-pyrrolo[1,2-b]pyrazole]-1-carboxamide
2′-[6-amino-5-(difluoromethoxy)pyridin-3-yl]-N-[1-(2-chlorophenyl)cyclobutyl]-5′,6′-dihydrospiro[azetidine-3,4′-pyrrolo[1,2-b]pyrazole]-1-carboxamide
2′-[6-amino-5-(trifluoromethoxy)pyridin-3-yl]-N-[(1R)-1-(4-cyanophenyl)ethyl]-5′,6′-dihydrospiro[azetidine-3,4′-pyrrolo[1,2-b]pyrazole]-1-carboxamide
2′-[6-amino-5-(difluoromethoxy)pyridin-3-yl]-N-[(1R)-1-(1,3,4-trimethyl-1H-pyrazol-5-yl)ethyl]-5′,6′-dihydrospiro[azetidine-3,4′-pyrrolo[1,2-b]pyrazole]-1-carboxamide
(rac)-2′-[6-amino-5-(trifluoromethoxy)pyridin-3-yl]-N-[1-(2-chlorophenyl)cyclobutyl]-5′,6′-dihydrospiro[pyrrolidine-3,4′-pyrrolo[1,2-b]pyrazole]-1-carboxamide
2′-[6-amino-5-(difluoromethoxy)pyridin-3-yl]-N-[(1R)-1-(5-fluoropyridin-3-yl)ethyl]-5′,6′-dihydrospiro[azetidine-3,4′-pyrrolo[1,2-b]pyrazole]-1-carboxamide
2′-[6-amino-5-(trifluoromethoxy)pyridin-3-yl]-N-[(1R)-1-(4-fluorophenyl)ethyl]-5′,6′-dihydrospiro[azetidine-3,4′-pyrrolo[1,2-b]pyrazole]-1-carboxamide
2′-[6-amino-5-(trifluoromethoxy)pyridin-3-yl]-N-[(1R)-1-(2-chloro-4-cyanophenyl)ethyl]-5′,6′-dihydrospiro[pyrrolidine-3,4′-pyrrolo[1,2-b]pyrazole]-1-carboxamide
2′-[6-amino-5-(difluoromethoxy)pyridin-3-yl]-N-[(1R)-1-(2-cyanophenyl)ethyl]-5′,6′-dihydrospiro[azetidine-3,4′-pyrrolo[1,2-b]pyrazole]-1-carboxamide
2′-[6-amino-5-(difluoromethoxy)pyridin-3-yl]-N-[2-(pyridin-4-yl)propan-2-yl]-5′,6′-dihydrospiro[azetidine-3,4′-pyrrolo[1,2-b]pyrazole]-1-carboxamide
2′-[6-amino-5-(difluoromethoxy)pyridin-3-yl]-N-[(1R)-1-(4-fluorophenyl)ethyl]-5′,6′-dihydrospiro[azetidine-3,4′-pyrrolo[1,2-b]pyrazole]-1-carboxamide
2′-[6-amino-5-(difluoromethoxy)pyridin-3-yl]-N-[(1R)-1-(2-cyanopyridin-4-yl)ethyl]-5′,6′-dihydrospiro[azetidine-3,4′-pyrrolo[1,2-b]pyrazole]-1-carboxamide
(rac)-2′-[6-amino-5-(trifluoromethoxy)pyridin-3-yl]-N-[1-(2-fluorophenyl)cyclobutyl]-5′,6′-dihydrospiro[pyrrolidine-3,4′-pyrrolo[1,2-b]pyrazole]-1-carboxamide
(rac)-2-[6-amino-5-(trifluoromethoxy)pyridin-3-yl]-N-[2-(2-chlorophenyl)propan-2-yl]-6,7-dihydrospiro[pyrazolo[5,1-c][1,4]oxazine-4,3′-pyrrolidine]-1′-carboxamide
(rac)-2-[6-amino-5-(trifluoromethoxy)pyridin-3-yl]-N-[2-(4-fluorophenyl)propan-2-yl]-6,7-dihydrospiro[pyrazolo[5,1-c][1,4]oxazine-4,3′-pyrrolidine]-1′-carboxamide
(rac)-2′-[6-amino-5-(trifluoromethoxy)pyridin-3-yl]-N-[2-(3-fluorophenyl)propan-2-yl]-5′,6′-dihydrospiro[pyrrolidine-3,4′-pyrrolo[1,2-b]pyrazole]-1-carboxamide
2′-[6-amino-5-(trifluoromethoxy)pyridin-3-yl]-N-[(1S)-1-(3-chloropyridin-4-yl)ethyl]-5′,6′-dihydrospiro[pyrrolidine-3,4′-pyrrolo[1,2-b]pyrazole]-1-carboxamide
2′-[6-amino-5-(difluoromethoxy)pyridin-3-yl]-N-[2-(4-fluorophenyl)propan-2-yl]-5′,6′-dihydrospiro[azetidine-3,4′-pyrrolo[1,2-b]pyrazole]-1-carboxamide
2′-[6-amino-5-(difluoromethoxy)pyridin-3-yl]-N-[2-(2-chlorophenyl)propan-2-yl]-5′,6′-dihydrospiro[azetidine-3,4′-pyrrolo[1,2-b]pyrazole]-1-carboxamide
2-[6-amino-5-(trifluoromethoxy)pyridin-3-yl]-N-[(1R)-1-(3-fluorophenyl)ethyl]-6,7-dihydrospiro[pyrazolo[5,1-c][1,4]oxazine-4,3′-pyrrolidine]-1′-carboxamide
2′-[6-amino-5-(trifluoromethoxy)pyridin-3-yl]-N-(1-phenylcyclobutyl)-5′,6′-dihydrospiro[azetidine-3,4′-pyrrolo[1,2-b]pyrazole]-1-carboxamide
2′-[6-amino-5-(trifluoromethoxy)pyridin-3-yl]-N-benzyl-5′,6′-dihydrospiro[azetidine-3,4′-pyrrolo[1,2-b]pyrazole]-1-carboxamide
2′-[6-amino-5-(trifluoromethoxy)pyridin-3-yl]-N-[(1R)-1-(4-cyanopyridin-2-yl)ethyl]-5′,6′-dihydrospiro[azetidine-3,4′-pyrrolo[1,2-b]pyrazole]-1-carboxamide
2′-[6-amino-5-(trifluoromethoxy)pyridin-3-yl]-N-[(1R)-1-(2-cyanopyridin-4-yl)ethyl]-5′,6′-dihydrospiro[azetidine-3,4′-pyrrolo[1,2-b]pyrazole]-1-carboxamide
2′-[6-amino-5-(trifluoromethoxy)pyridin-3-yl]-N-[(1R)-1-(5-fluoropyridin-3-yl)ethyl]-5′,6′-dihydrospiro[pyrrolidine-3,4′-pyrrolo[1,2-b]pyrazole]-1-carboxamide
(rac)-2′-[6-amino-5-(trifluoromethoxy)pyridin-3-yl]-N-[2-(pyridin-4-yl)propan-2-yl]-5′,6′-dihydrospiro[pyrrolidine-3,4′-pyrrolo[1,2-b]pyrazole]-1-carboxamide
(rac)-2′-[6-amino-5-(trifluoromethoxy)pyridin-3-yl]-N-(1-phenylcyclobutyl)-5′,6′-dihydrospiro[pyrrolidine-3,4′-pyrrolo[1,2-b]pyrazole]-1-carboxamide
(rac)-2′-[6-amino-5-(trifluoromethoxy)pyridin-3-yl]-N-[1-(pyridin-4-yl)cyclobutyl]-5′,6′-dihydrospiro[pyrrolidine-3,4′-pyrrolo[1,2-b]pyrazole]-1-carboxamide
2′-[6-amino-5-(difluoromethoxy)pyridin-3-yl]-N-[2-(3-fluorophenyl)propan-2-yl]-5′,6′-dihydrospiro[azetidine-3,4′-pyrrolo[1,2-b]pyrazole]-1-carboxamide
2′-[6-amino-5-(difluoromethoxy)pyridin-3-yl]-N-[(1S)-1-(3-chloropyridin-4-yl)ethyl]-5′,6′-dihydrospiro[azetidine-3,4′-pyrrolo[1,2-b]pyrazole]-1-carboxamide
2′-[6-amino-5-(difluoromethoxy)pyridin-3-yl]-N-[2-(3-chloropyridin-4-yl)propan-2-yl]-5′,6′-dihydrospiro[azetidine-3,4′-pyrrolo[1,2-b]pyrazole]-1-carboxamide
2′-[6-amino-5-(difluoromethoxy)pyridin-3-yl]-N-[(1R)-1-(2-chloro-4-cyanophenyl)ethyl]-5′,6′-dihydrospiro[azetidine-3,4′-pyrrolo[1,2-b]pyrazole]-1-carboxamide
2′-[6-amino-5-(difluoromethoxy)pyridin-3-yl]-N-[1-(2-fluorophenyl)cyclobutyl]-5′,6′-dihydrospiro[azetidine-3,4′-pyrrolo[1,2-b]pyrazole]-1-carboxamide
(rac)-2′-[6-amino-5-(trifluoromethoxy)pyridin-3-yl]-N-[2-(2-chlorophenyl)propan-2-yl]-5′,6′-dihydrospiro[pyrrolidine-3,4′-pyrrolo[1,2-b]pyrazole]-1-carboxamide
2-[6-amino-5-(trifluoromethoxy)pyridin-3-yl]-N-[(1R)-1-(4-cyanopyridin-2-yl)ethyl]-6,7-dihydrospiro[pyrazolo[5,1-c][1,4]oxazine-4,3′-pyrrolidine]-1′-carboxamide
(rac)-2-[6-amino-5-(difluoromethoxy)pyridin-3-yl]-N-[1-(2-chlorophenyl)cyclobutyl]-6,7-dihydrospiro[pyrazolo[5,1-c][1,4]oxazine-4,3′-pyrrolidine]-1′-carboxamide
(rac)-2-[6-amino-5-(trifluoromethoxy)pyridin-3-yl]-N-[1-(pyridin-2-yl)cyclobutyl]-6,7-dihydrospiro[pyrazolo[5,1-c][1,4]oxazine-4,3′-pyrrolidine]-1′-carboxamide
2-[6-amino-5-(trifluoromethoxy)pyridin-3-yl]-N-[(1R)-1-(2-cyanopyridin-4-yl)ethyl]-6,7-dihydrospiro[pyrazolo[5,1-c][1,4]oxazine-4,3′-pyrrolidine]-1′-carboxamide
(rac)-2-[6-amino-5-(difluoromethoxy)pyridin-3-yl]-N-[2-(2-chlorophenyl)propan-2-yl]-6,7-dihydrospiro[pyrazolo[5,1-c][1,4]oxazine-4,3′-pyrrolidine]-1′-carboxamide
(rac)-2-[6-amino-5-(trifluoromethoxy)pyridin-3-yl]-N-[1-(2-chlorophenyl)cyclobutyl]-6,7-dihydrospiro[pyrazolo[5,1-c][1,4]oxazine-4,3′-pyrrolidine]-1′-carboxamide
(rac)-2-[6-amino-5-(trifluoromethoxy)pyridin-3-yl]-N-[1-(2-fluorophenyl)cyclobutyl]-6,7-dihydrospiro[pyrazolo[5,1-c][1,4]oxazine-4,3′-pyrrolidine]-1′-carboxamide
(rac)-2-[6-amino-5-(trifluoromethoxy)pyridin-3-yl]-N-[2-(3-chloropyridin-4-yl)propan-2-yl]-6,7-dihydrospiro[pyrazolo[5,1-c][1,4]oxazine-4,3′-pyrrolidine]-1′-carboxamide
(rac)-2′-[6-amino-5-(trifluoromethoxy)pyridin-3-yl]-N-[1-(pyridin-4-yl)cyclobutyl]-5′,6′-dihydro-1H-spiro[pyrrolidine-3,4′-pyrrolo[1,2-b]pyrazole]-1-carboxamide acetate (1:1)
(rac)-2-[6-amino-5-(trifluoromethoxy)pyridin-3-yl]-N-[(1-methyl-2-oxabicyclo[2.1.1]hexan-4-yl)methyl]-6,7-dihydrospiro[pyrazolo[5,1-c][1,4]oxazine-4,3′-pyrrolidine]-1′-carboxamide
(rac)-2-[6-amino-5-(trifluoromethoxy)pyridin-3-yl]-N-[2-(2-fluorophenyl)propan-2-yl]-6,7-dihydrospiro[pyrazolo[5,1-c][1,4]oxazine-4,3′-pyrrolidine]-1′-carboxamide
2-[6-amino-5-(trifluoromethoxy)pyridin-3-yl]-N-[(1R)-1-(2-chloro-4-cyanophenyl)ethyl]-6,7-dihydrospiro[pyrazolo[5,1-c][1,4]oxazine-4,3′-pyrrolidine]-1′-carboxamide
2′-[6-amino-5-(difluoromethoxy)pyridin-3-yl]-N-[2-(pyridin-2-yl)propan-2-yl]-5′,6′-dihydrospiro[azetidine-3,4′-pyrrolo[1,2-b]pyrazole]-1-carboxamide
2′-[6-amino-5-(difluoromethoxy)pyridin-3-yl]-N-[2-(1-methyl-1H-1,2,3-triazol-4-yl)propan-2-yl]-5′,6′-dihydrospiro[azetidine-3,4′-pyrrolo[1,2-b]pyrazole]-1-carboxamide
2-[6-amino-5-(trifluoromethoxy)pyridin-3-yl]-N-[(1S)-2-methoxy-1-phenylethyl]-6,7-dihydrospiro[pyrazolo[5,1-c][1,4]oxazine-4,3′-pyrrolidine]-1′-carboxamide
2-[6-amino-5-(trifluoromethoxy)pyridin-3-yl]-N-[(1S)-1-(3-chloropyridin-4-yl)ethyl]-6,7-dihydrospiro[pyrazolo[5,1-c][1,4]oxazine-4,3′-pyrrolidine]-1′-carboxamide
(rac)-2-[6-amino-5-(trifluoromethoxy)pyridin-3-yl]-N-[2-(3-fluorophenyl)propan-2-yl]-6,7-dihydrospiro[pyrazolo[5,1-c][1,4]oxazine-4,3′-pyrrolidine]-1′-carboxamide
(rac)-2′-[6-amino-5-(trifluoromethoxy)pyridin-3-yl]-N-[(1-methyl-2-oxabicyclo[2.1.1]hexan-4-yl)methyl]-5′,6′-dihydrospiro[pyrrolidine-3,4′-pyrrolo[1,2-b]pyrazole]-1-carboxamide
2′-[6-amino-5-(difluoromethoxy)pyridin-3-yl]-N-[(1R)-1-phenylpropyl]-5′,6′-dihydrospiro[azetidine-3,4′-pyrrolo[1,2-b]pyrazole]-1-carboxamide
2′-[6-amino-5-(trifluoromethoxy)pyridin-3-yl]-N-[(1S)-2-methoxy-1-phenylethyl]-5′,6′-dihydrospiro[pyrrolidine-3,4′-pyrrolo[1,2-b]pyrazole]-1-carboxamide
2′-[6-amino-5-(difluoromethoxy)pyridin-3-yl]-N-[(1R)-1-(3-fluorophenyl)ethyl]-5′,6′-dihydrospiro[azetidine-3,4′-pyrrolo[1,2-b]pyrazole]-1-carboxamide
2′-[6-amino-5-(trifluoromethoxy)pyridin-3-yl]-N-[(1S)-1-(pyridin-3-yl)ethyl]-5′,6′-dihydrospiro[pyrrolidine-3,4′-pyrrolo[1,2-b]pyrazole]-1-carboxamide
(rac)-2′-[6-amino-5-(trifluoromethoxy)pyridin-3-yl]-N-[1-(pyridin-3-yl)cyclobutyl]-5′,6′-dihydrospiro[pyrrolidine-3,4′-pyrrolo[1,2-b]pyrazole]-1-carboxamide
(rac)-2′-[6-amino-5-(trifluoromethoxy)pyridin-3-yl]-N-[1-(3-fluorophenyl)cyclobutyl]-5′,6′-dihydrospiro[pyrrolidine-3,4′-pyrrolo[1,2-b]pyrazole]-1-carboxamide
2′-[6-amino-5-(difluoromethoxy)pyridin-3-yl]-N-[1-(propan-2-yl)cyclobutyl]-5′,6′-dihydrospiro[azetidine-3,4′-pyrrolo[1,2-b]pyrazole]-1-carboxamide
2′-[6-amino-5-(difluoromethoxy)pyridin-3-yl]-N-[1-(pyridin-4-yl)cyclobutyl]-5′,6′-dihydrospiro[azetidine-3,4′-pyrrolo[1,2-b]pyrazole]-1-carboxamide
2′-[6-amino-5-(difluoromethoxy)pyridin-3-yl]-N-(bicyclo[2.2.1]heptan-1-yl)-5′,6′-dihydrospiro[azetidine-3,4′-pyrrolo[1,2-b]pyrazole]-1-carboxamide
2′-[6-amino-5-(difluoromethoxy)pyridin-3-yl]-N-(1-benzylcyclobutyl)-5′,6′-dihydrospiro[azetidine-3,4′-pyrrolo[1,2-b]pyrazole]-1-carboxamide
2′-[6-amino-5-(difluoromethoxy)pyridin-3-yl]-N-[1-(4-chlorophenyl)cyclobutyl]-5′,6′-dihydrospiro[azetidine-3,4′-pyrrolo[1,2-b]pyrazole]-1-carboxamide
2′-[6-amino-5-(difluoromethoxy)pyridin-3-yl]-N-(1-methylcyclobutyl)-5′,6′-dihydrospiro[azetidine-3,4′-pyrrolo[1,2-b]pyrazole]-1-carboxamide
(rac)-2-[6-amino-5-(trifluoromethoxy)pyridin-3-yl]-N-[1-(1,3,4-trimethyl-1H-pyrazol-5-yl)ethyl]-6,7-dihydrospiro[pyrazolo[5,1-c][1,4]oxazine-4,3′-pyrrolidine]-1′-carboxamide
2-[6-amino-5-(trifluoromethoxy)pyridin-3-yl]-N-[(1R)-2,3-dihydro-1H-inden-1-yl]-6,7-dihydrospiro[pyrazolo[5,1-c][1,4]oxazine-4,3′-pyrrolidine]-1′-carboxamide
2′-[6-amino-5-(difluoromethoxy)pyridin-3-yl]-N-[1-(pyridin-2-yl)cyclobutyl]-5′,6′-dihydrospiro[azetidine-3,4′-pyrrolo[1,2-b]pyrazole]-1-carboxamide
2′-[6-amino-5-(difluoromethoxy)pyridin-3-yl]-N-[1-(3-fluorophenyl)cyclobutyl]-5′,6′-dihydrospiro[azetidine-3,4′-pyrrolo[1,2-b]pyrazole]-1-carboxamide
(rac)-2′-[6-amino-5-(trifluoromethoxy)pyridin-3-yl]-N-[1-(pyridin-2-yl)cyclobutyl]-5′,6′-dihydrospiro[pyrrolidine-3,4′-pyrrolo[1,2-b]pyrazole]-1-carboxamide
2-[6-amino-5-(difluoromethoxy)pyridin-3-yl]-N-[(1S)-2-methoxy-1-phenylethyl]-6,7-dihydrospiro[pyrazolo[5,1-c][1,4]oxazine-4,3′-pyrrolidine]-1′-carboxamide
2′-[6-amino-5-(difluoromethoxy)pyridin-3-yl]-N-[(1R)-1-phenylethyl]-5′,6′-dihydrospiro[pyrrolidine-3,4′-pyrrolo[1,2-b]pyrazole]-1-carboxamide
(rac)-2′-[6-amino-5-(difluoromethoxy)pyridin-3-yl]-N-[1-(pyridin-4-yl)cyclobutyl]-5′,6′-dihydrospiro[pyrrolidine-3,4′-pyrrolo[1,2-b]pyrazole]-1-carboxamide
(rac)-2′-[6-amino-5-(difluoromethoxy)pyridin-3-yl]-N-(1-phenylcyclobutyl)-5′,6′-dihydrospiro[pyrrolidine-3,4′-pyrrolo[1,2-b]pyrazole]-1-carboxamide
(rac)-2′-[6-amino-5-(difluoromethoxy)pyridin-3-yl]-N-[2-(pyridin-4-yl)propan-2-yl]-5′,6′-dihydrospiro[pyrrolidine-3,4′-pyrrolo[1,2-b]pyrazole]-1-carboxamide
2′-[6-amino-5-(difluoromethoxy)pyridin-3-yl]-N-[(1R)-1-(4-fluorophenyl)ethyl]-5′,6′-dihydrospiro[pyrrolidine-3,4′-pyrrolo[1,2-b]pyrazole]-1-carboxamide
(rac)-2′-[6-amino-5-(difluoromethoxy)pyridin-3-yl]-N-[2-(4-fluorophenyl)propan-2-yl]-5′,6′-dihydrospiro[pyrrolidine-3,4′-pyrrolo[1,2-b]pyrazole]-1-carboxamide
2′-[6-amino-5-(difluoromethoxy)pyridin-3-yl]-N-[(1R)-1-(5-fluoropyridin-3-yl)ethyl]-5′,6′-dihydrospiro[pyrrolidine-3,4′-pyrrolo[1,2-b]pyrazole]-1-carboxamide
2′-{6-amino-5-[(1R)-1-(pyridin-2-yl)ethoxy]pyridin-3-yl}-N-[(1R)-1-phenylethyl]-5′,6′-dihydrospiro[pyrrolidine-3,4′-pyrrolo[1,2-b]pyrazole]-1-carboxamide
2′-{6-amino-5-[(1R)-1-(pyridin-2-yl)ethoxy]pyridin-3-yl}-N-[2-(pyridin-4-yl)propan-2-yl]-5′,6′-dihydrospiro[pyrrolidine-3,4′-pyrrolo[1,2-b]pyrazole]-1-carboxamide
2′-{6-amino-5-[(1S)-1-(pyridin-2-yl)ethoxy]pyridin-3-yl}-N-[(1R)-1-phenylethyl]-5′,6′-dihydrospiro[pyrrolidine-3,4′-pyrrolo[1,2-b]pyrazole]-1-carboxamide
2′-{6-amino-5-[(1S)-1-(pyridin-2-yl)ethoxy]pyridin-3-yl}-N-[2-(pyridin-4-yl)propan-2-yl]-5′,6′-dihydrospiro[pyrrolidine-3,4′-pyrrolo[1,2-b]pyrazole]-1-carboxamide
2′-{6-amino-5-[(trifluoromethyl)sulfanyl]pyridin-3-yl}-N-[(1R)-1-(3-fluorophenyl)ethyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(3R)-2,3-dihydro-1-benzofuran-3-yl]-5′,6′-dihydrospiro[pyrrolidine-3,4′-pyrrolo[1,2-b]pyrazole]-1-carboxamide
2′-{6-amino-5-[(1R)-1-(pyridin-3-yl)ethoxy]pyridin-3-yl}-N-[2-(pyridin-4-yl)propan-2-yl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[1-(1-methyl-1H-imidazol-5-yl)ethyl]-5′,6′-dihydrospiro[pyrrolidine-3,4′-pyrrolo[1,2-b]pyrazole]-1-carboxamide (diastereomeric mixture)
2′-{6-amino-5-[(1R)-1-(pyridin-3-yl)ethoxy]pyridin-3-yl}-N-cyclobutyl-5′,6′-dihydrospiro[pyrrolidine-3,4′-pyrrolo[1,2-b]pyrazole]-1-carboxamide
2′-{6-amino-5-[(1R)-1-(pyridin-3-yl)ethoxy]pyridin-3-yl}-N-[2-(1-methyl-1H-1,2,3-triazol-4-yl)propan-2-yl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[cyclopropyl(pyridin-2-yl)methyl]-5′,6′-dihydrospiro[pyrrolidine-3,4′-pyrrolo[1,2-b]pyrazole]-1-carboxamide (diastereomeric mixture)
2′-{6-amino-5-[(1S)-1-(pyridin-3-yl)ethoxy]pyridin-3-yl}-N-cyclobutyl-5′,6′-dihydrospiro[pyrrolidine-3,4′-pyrrolo[1,2-b]pyrazole]-1-carboxamide
2′-{6-amino-5-[(1S)-1-(pyridin-3-yl)ethoxy]pyridin-3-yl}-N-[2-(pyridin-4-yl)propan-2-yl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[2-(2-cyanophenyl)propan-2-yl]-5′,6′-dihydrospiro[pyrrolidine-3,4′-pyrrolo[1,2-b]pyrazole]-1-carboxamide
[2′-{6-amino-5-[(1R)-1-(pyridin-3-yl)ethoxy]pyridin-3-yl}-5′,6′-dihydrospiro[pyrrolidine-3,4′-pyrrolo[1,2-b]pyrazol]-1-yl](1H-imidazol-1-yl)methanone
(rac)-2′-[6-amino-5-(trifluoromethyl)pyridin-3-yl]-N-(1-methylcyclobutyl)-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-(3-methyloxetan-3-yl)-5′,6′-dihydrospiro[pyrrolidine-3,4′-pyrrolo[1,2-b]pyrazole]-1-carboxamide
(rac)-2-[6-amino-5-(trifluoromethoxy)pyridin-3-yl]-N-[2-(3-fluoropyridin-2-yl)propan-2-yl]-6,7-dihydrospiro[pyrazolo[5,1-c][1,4]oxazine-4,3′-pyrrolidine]-1′-carboxamide
2′-{6-amino-5-[(1S)-1-phenylethoxy]pyridin-3-yl}-N-[1-(pyridin-4-yl)cyclobutyl]-5′,6′-dihydrospiro[pyrrolidine-3,4′-pyrrolo[1,2-b]pyrazole]-1-carboxamide
2′-{6-amino-5-[(1S)-1-phenylethoxy]pyridin-3-yl}-N-[2-(3-chloropyridin-4-yl)propan-2-yl]-5′,6′-dihydrospiro[pyrrolidine-3,4′-pyrrolo[1,2-b]pyrazole]-1-carboxamide
2′-{6-amino-5-[(1R)-1-phenylethoxy]pyridin-3-yl}-N-[1-(pyridin-4-yl)cyclobutyl]-5′,6′-dihydrospiro[pyrrolidine-3,4′-pyrrolo[1,2-b]pyrazole]-1-carboxamide
2′-{6-amino-5-[(1R)-1-phenylethoxy]pyridin-3-yl}-N-[2-(3-chloropyridin-4-yl)propan-2-yl]-5′,6′-dihydrospiro[pyrrolidine-3,4′-pyrrolo[1,2-b]pyrazole]-1-carboxamide
(rac)-2-[6-amino-5-(trifluoromethoxy)pyridin-3-yl]-N-[2-(pyridin-2-yl)propan-2-yl]-6,7-dihydrospiro[pyrazolo[5,1-c][1,4]oxazine-4,3′-pyrrolidine]-1′-carboxamide
2′-{6-amino-5-[(1R)-1-(pyridin-2-yl)ethoxy]pyridin-3-yl}-N-[2-(3-chloropyridin-4-yl)propan-2-yl]-5′,6′-dihydrospiro[pyrrolidine-3,4′-pyrrolo[1,2-b]pyrazole]-1-carboxamide
2′-{6-amino-5-[(1R)-1-(pyridin-3-yl)ethoxy]pyridin-3-yl}-N-[2-(3-chloropyridin-4-yl)propan-2-yl]-5′,6′-dihydrospiro[pyrrolidine-3,4′-pyrrolo[1,2-b]pyrazole]-1-carboxamide
2′-{6-amino-5-[(1R)-1-(pyridin-4-yl)ethoxy]pyridin-3-yl}-N-[2-(3-chloropyridin-4-yl)propan-2-yl]-5′,6′-dihydrospiro[pyrrolidine-3,4′-pyrrolo[1,2-b]pyrazole]-1-carboxamide
2′-(6-amino-5-cyanopyridin-3-yl)-N-[(1R)-1-(pyridin-4-yl)ethyl]-5′,6′-dihydrospiro[pyrrolidine-3,4′-pyrrolo[1,2-b]pyrazole]-1-carboxamide (isomer 1)
2′-(6-amino-5-cyanopyridin-3-yl)-N-[(1R)-1-(pyridin-4-yl)ethyl]-5′,6′-dihydrospiro[pyrrolidine-3,4′-pyrrolo[1,2-b]pyrazole]-1-carboxamide (isomer 2)
2′-(6-amino-5-cyanopyridin-3-yl)-N-[1-(pyridin-4-yl)cyclobutyl]-5′,6′-dihydrospiro[pyrrolidine-3,4′-pyrrolo[1,2-b]pyrazole]-1-carboxamide (enantiomer 1)
(3R)-2′-[6-amino-5-(trifluoromethyl)pyridin-3-yl]-N-[(1S)-1-(pyridin-4-yl)ethyl]-5′,6′-dihydrospiro[pyrrolidine-3,4′-pyrrolo[1,2-b]pyrazole]-1-carboxamide
(3S)-2′-[6-amino-5-(trifluoromethyl)pyridin-3-yl]-N-[(1S)-1-(pyridin-4-yl)ethyl]-5′,6′-dihydrospiro[pyrrolidine-3,4′-pyrrolo[1,2-b]pyrazole]-1-carboxamide
2′-(6-amino-5-cyanopyridin-3-yl)-N-[(1S)-1-(pyridin-4-yl)ethyl]-5′,6′-dihydrospiro[pyrrolidine-3,4′-pyrrolo[1,2-b]pyrazole]-1-carboxamide (isomer 1)
2′-(6-amino-5-cyanopyridin-3-yl)-N-[(1S)-1-(pyridin-4-yl)ethyl]-5′,6′-dihydrospiro[pyrrolidine-3,4′-pyrrolo[1,2-b]pyrazole]-1-carboxamide (isomer 2)
2′-[6-amino-5-(trifluoromethyl)pyridin-3-yl]-N-[(1S)-1-(pyridin-4-yl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(1R)-1-(pyridin-4-yl)ethyl]-5′,6′-dihydrospiro[azetidine-3,4′-pyrrolo[1,2-b]pyrazole]-1-carboxamide
(rac)-2′-[6-amino-5-(trifluoromethyl)pyridin-3-yl]-N-[1-(2-chlorophenyl)cyclobutyl]-5′,6′-dihydrospiro[pyrrolidine-3,4′-pyrrolo[1,2-b]pyrazole]-1-carboxamide
(rac)-2-[6-amino-5-(trifluoromethyl)pyridin-3-yl]-N-[2-(2-fluorophenyl)propan-2-yl]-6,7-dihydrospiro[pyrazolo[5,1-c][1,4]oxazine-4,3′-pyrrolidine]-1′-carboxamide
(rac)-2-[6-amino-5-(trifluoromethyl)pyridin-3-yl]-N-(1-phenylcyclobutyl)-6,7-dihydrospiro[pyrazolo[5,1-c][1,4]oxazine-4,3′-pyrrolidine]-1′-carboxamide
(rac)-2′-[6-amino-5-(trifluoromethyl)pyridin-3-yl]-N-[2-(4-fluorophenyl)propan-2-yl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[2-(2-chlorophenyl)propan-2-yl]-5′,6′-dihydrospiro[pyrrolidine-3,4′-pyrrolo[1,2-b]pyrazole]-1-carboxamide
2-[6-amino-5-(trifluoromethyl)pyridin-3-yl]-N-[(1R)-1-(2-chloro-4-cyanophenyl)ethyl]-6,7-dihydrospiro[pyrazolo[5,1-c][1,4]oxazine-4,3′-pyrrolidine]-1′-carboxamide (mixture of stereoisomer)
2′-[6-amino-5-(trifluoromethyl)pyridin-3-yl]-N-[(1R)-1-(2-chloro-4-cyanophenyl)ethyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1S)-1-(3-chloropyridin-4-yl)ethyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[2-(3-fluorophenyl)propan-2-yl]-5′,6′-dihydrospiro[pyrrolidine-3,4′-pyrrolo[1,2-b]pyrazole]-1-carboxamide
(rac)-2-[6-amino-5-(trifluoromethyl)pyridin-3-yl]-N-[1-(pyridin-4-yl)cyclobutyl]-6,7-dihydrospiro[pyrazolo[5,1-c][1,4]oxazine-4,3′-pyrrolidine]-1′-carboxamide
(rac)-2′-[6-amino-5-(trifluoromethyl)pyridin-3-yl]-N-[2-(3-chloropyridin-4-yl)propan-2-yl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-(1-phenylcyclobutyl)-5′,6′-dihydrospiro[pyrrolidine-3,4′-pyrrolo[1,2-b]pyrazole]-1-carboxamide
(rac)-2-[6-amino-5-(trifluoromethyl)pyridin-3-yl]-N-[2-(pyridin-2-yl)propan-2-yl]-6,7-dihydrospiro[pyrazolo[5,1-c][1,4]oxazine-4,3′-pyrrolidine]-1′-carboxamide
(rac)-2-[6-amino-5-(trifluoromethyl)pyridin-3-yl]-N-[1-(2-fluorophenyl)cyclobutyl]-6,7-dihydrospiro[pyrazolo[5,1-c][1,4]oxazine-4,3′-pyrrolidine]-1′-carboxamide
(rac)-2′-[6-amino-5-(trifluoromethyl)pyridin-3-yl]-N-[1-(2-fluorophenyl)cyclobutyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[1-(3-fluorophenyl)cyclobutyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[1-(pyridin-4-yl)cyclobutyl]-5′,6′-dihydrospiro[pyrrolidine-3,4′-pyrrolo[1,2-b]pyrazole]-1-carboxamide
(rac)-2-[6-amino-5-(trifluoromethyl)pyridin-3-yl]-N-[1-(3-fluorophenyl)cyclobutyl]-6,7-dihydrospiro[pyrazolo[5,1-c][1,4]oxazine-4,3′-pyrrolidine]-1′-carboxamide
(3′R)-2-[6-amino-5-(trifluoromethoxy)pyridin-3-yl]-N-[2-(pyridin-2-yl)propan-2-yl]-6,7-dihydrospiro[pyrazolo[5,1-c][1,4]oxazine-4,3′-pyrrolidine]-1′-carboxamide
(3′R)-2-[6-amino-5-(trifluoromethyl)pyridin-3-yl]-N-[2-(pyridin-3-yl)propan-2-yl]-6,7-dihydrospiro[pyrazolo[5,1-c][1,4]oxazine-4,3′-pyrrolidine]-1′-carboxamide
(3R)-2′-[6-amino-5-(trifluoromethyl)pyridin-3-yl]-N-[2-(pyridin-4-yl)propan-2-yl]-5′,6′-dihydrospiro[pyrrolidine-3,4′-pyrrolo[1,2-b]pyrazole]-1-carboxamide
(3′R)-2-[6-amino-5-(trifluoromethyl)pyridin-3-yl]-N-[2-(pyridin-4-yl)propan-2-yl]-6,7-dihydrospiro[pyrazolo[5,1-c][1,4]oxazine-4,3′-pyrrolidine]-1′-carboxamide
(3R)-2′-[6-amino-5-(trifluoromethyl)pyridin-3-yl]-N-[2-(pyridin-3-yl)propan-2-yl]-5′,6′-dihydrospiro[pyrrolidine-3,4′-pyrrolo[1,2-b]pyrazole]-1-carboxamide
(3R)-2′-[6-amino-5-(difluoromethoxy)pyridin-3-yl]-N-[(1R)-1-phenylethyl]-5′,6′-dihydrospiro[pyrrolidine-3,4′-pyrrolo[1,2-b]pyrazole]-1-carboxamide
(3′R)-2-[6-amino-5-(difluoromethoxy)pyridin-3-yl]-N-[(1R)-1-phenylethyl]-6,7-dihydrospiro[pyrazolo[5,1-c][1,4]oxazine-4,3′-pyrrolidine]-1′-carboxamide
(3R)-2′-[6-amino-5-(trifluoromethyl)pyridin-3-yl]-N-[2-(pyridin-2-yl)propan-2-yl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1R)-1-phenylethyl]-5′,6′-dihydrospiro[pyrrolidine-3,4′-pyrrolo[1,2-b]pyrazole]-1-carboxamide (stereoisomer 1)
2′-[6-amino-5-(trifluoromethyl)pyridin-3-yl]-N-[(1R)-1-phenylethyl]-5′,6′-dihydrospiro[pyrrolidine-3,4′-pyrrolo[1,2-b]pyrazole]-1-carboxamide (stereoisomer 2)
(rac)-2′-[6-amino-5-(trifluoromethyl)pyridin-3-yl]-N-[2-(3,5-difluorophenyl)propan-2-yl]-5′,6′-dihydrospiro[pyrrolidine-3,4′-pyrrolo[1,2-b]pyrazole]-1-carboxamide
(rac)-1-{2′-[6-amino-5-(trifluoromethyl) pyridin-3-yl]-5′,6′-dihydrospiro[pyrrolidine-3,4′-pyrrolo[1,2-b]pyrazol]-1-yl}-2-(5-fluoropyridin-2-yl)-2-methylpropan-1-one
(rac)-2′-[6-amino-5-(trifluoromethyl)pyridin-3-yl]-N-[2-(6-methylpyridin-3-yl)propan-2-yl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-(1-phenylcyclohexyl)-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[2-(5-fluoropyridin-2-yl)propan-2-yl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1R)-2,2,2-trifluoro-1-methoxy-1-phenylethyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[2-(pyrimidin-4-yl)propan-2-yl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[2-(pyrimidin-5-yl)propan-2-yl]-5′,6′-dihydrospiro[pyrrolidine-3,4′-pyrrolo[1,2-b]pyrazole]-1-carboxamide
(rac)-{2′-[6-amino-5-(trifluoromethyl) pyridin-3-yl]-5′,6′-dihydrospiro[pyrrolidine-3,4′-pyrrolo[1,2-b]pyrazol]-1-yl}(3-phenyloxetan-3-yl)methanone
(rac)-1-{2′-[6-amino-5-(trifluoromethyl) pyridin-3-yl]-5′,6′-dihydrospiro[pyrrolidine-3,4′-pyrrolo[1,2-b]pyrazol]-1-yl}-2-(3,5-difluorophenyl)-2-methylpropan-1-one
(rac)-1-{2′-[6-amino-5-(trifluoromethyl) pyridin-3-yl]-5′,6′-dihydrospiro[pyrrolidine-3,4′-pyrrolo[1,2-b]pyrazol]-1-yl}-2-methyl-2-(6-methylpyridin-3-yl)propan-1-one
(rac)-2′-[6-amino-5-(trifluoromethyl)pyridin-3-yl]-N-(3-phenyloxetan-3-yl)-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-(propan-2-yl)-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-cyclobutyl-5′,6′-dihydrospiro[pyrrolidine-3,4′-pyrrolo[1,2-b]pyrazole]-1-carboxamide
(3R)-2′-[6-amino-5-(trifluoromethyl)pyridin-3-yl]-N-ethyl-5′,6′-dihydrospiro[pyrrolidine-3,4′-pyrrolo[1,2-b]pyrazole]-1-carboxamide
(3S)-2′-[6-amino-5-(trifluoromethyl)pyridin-3-yl]-N-ethyl-5′,6′-dihydrospiro[pyrrolidine-3,4′-pyrrolo[1,2-b]pyrazole]-1-carboxamide
(3R)-2′-[6-amino-5-(trifluoromethyl)pyridin-3-yl]-N-(propan-2-yl)-5′,6′-dihydrospiro[pyrrolidine-3,4′-pyrrolo[1,2-b]pyrazole]-1-carboxamide
(3S)-2′-[6-amino-5-(trifluoromethyl)pyridin-3-yl]-N-(propan-2-yl)-5′,6′-dihydrospiro[pyrrolidine-3,4′-pyrrolo[1,2-b]pyrazole]-1-carboxamide
(rac)-2-[6-amino-5-(trifluoromethyl)pyridin-3-yl]-N-cyclobutyl-6,7-dihydrospiro[pyrazolo[5,1-c][1,4]oxazine-4,3′-pyrrolidine]-1′-carboxamide
(rac)-2-[6-amino-5-(trifluoromethyl)pyridin-3-yl]-N-ethyl-6,7-dihydrospiro[pyrazolo[5,1-c][1,4]oxazine-4,3′-pyrrolidine]-1′-carboxamide
(rac)-2-[6-amino-5-(trifluoromethyl)pyridin-3-yl]-N-(propan-2-yl)-6,7-dihydrospiro[pyrazolo[5,1-c][1,4]oxazine-4,3′-pyrrolidine]-1′-carboxamide
2′-[6-amino-5-(trifluoromethyl)pyridin-3-yl]-N-cyclobutyl-5′,6′-dihydrospiro[azetidine-3,4′-pyrrolo[1,2-b]pyrazole]-1-carboxamide
2′-[6-amino-5-(trifluoromethyl)pyridin-3-yl]-N-ethyl-5′,6′-dihydrospiro[azetidine-3,4′-pyrrolo[1,2-b]pyrazole]-1-carboxamide
2′-[6-amino-5-(trifluoromethyl)pyridin-3-yl]-N-(propan-2-yl)-5′,6′-dihydrospiro[azetidine-3,4′-pyrrolo[1,2-b]pyrazole]-1-carboxamide
(rac)-2′-(6-amino-5-cyanopyridin-3-yl)-N-[2-(3-chloropyridin-4-yl)propan-2-yl]-5′,6′-dihydrospiro[pyrrolidine-3,4′-pyrrolo[1,2-b]pyrazole]-1-carboxamide
(rac)-2′-{6-amino-5-[(2-chlorophenyl)methoxy]pyridin-3-yl}-N-ethyl-5′,6′-dihydrospiro[pyrrolidine-3,4′-pyrrolo[1,2-b]pyrazole]-1-carboxamide
(rac)-2′-{6-amino-5-[(2-chlorophenyl)methoxy]pyridin-3-yl}-N-(propan-2-yl)-5′,6′-dihydrospiro[pyrrolidine-3,4′-pyrrolo[1,2-b]pyrazole]-1-carboxamide
(rac)-2′-{6-amino-5-[(2-chlorophenyl)methoxy]pyridin-3-yl}-N-cyclobutyl-5′,6′-dihydrospiro[pyrrolidine-3,4′-pyrrolo[1,2-b]pyrazole]-1-carboxamide
(rac)-2′-{6-amino-5-[(2-fluorophenyl)methoxy]pyridin-3-yl}-N-ethyl-5′,6′-dihydrospiro[pyrrolidine-3,4′-pyrrolo[1,2-b]pyrazole]-1-carboxamide
(rac)-2′-{6-amino-5-[(2-fluorophenyl)methoxy]pyridin-3-yl}-N-(propan-2-yl)-5′,6′-dihydrospiro[pyrrolidine-3,4′-pyrrolo[1,2-b]pyrazole]-1-carboxamide
(rac)-2′-{6-amino-5-[(2-fluorophenyl)methoxy]pyridin-3-yl}-N-cyclobutyl-5′,6′-dihydrospiro[pyrrolidine-3,4′-pyrrolo[1,2-b]pyrazole]-1-carboxamide
(rac)-2′-{6-amino-5-[(2-fluorophenyl)methoxy]pyridin-3-yl}-N-benzyl-5′,6′-dihydrospiro[pyrrolidine-3,4′-pyrrolo[1,2-b]pyrazole]-1-carboxamide
(rac)-2′-{6-amino-5-[(3-fluorophenyl)methoxy]pyridin-3-yl}-N-ethyl-5′,6′-dihydrospiro[pyrrolidine-3,4′-pyrrolo[1,2-b]pyrazole]-1-carboxamide
(rac)-2′-{6-amino-5-[(3-fluorophenyl)methoxy]pyridin-3-yl}-N-(propan-2-yl)-5′,6′-dihydrospiro[pyrrolidine-3,4′-pyrrolo[1,2-b]pyrazole]-1-carboxamide
(rac)-2′-{6-amino-5-[(3-fluorophenyl)methoxy]pyridin-3-yl}-N-cyclobutyl-5′,6′-dihydrospiro[pyrrolidine-3,4′-pyrrolo[1,2-b]pyrazole]-1-carboxamide
(rac)-2′-{6-amino-5-[(3-fluorophenyl)methoxy]pyridin-3-yl}-N-benzyl-5′,6′-dihydrospiro[pyrrolidine-3,4′-pyrrolo[1,2-b]pyrazole]-1-carboxamide
(rac)-2′-{6-amino-5-[(pyridin-2-yl)methoxy]pyridin-3-yl}-N-ethyl-5′,6′-dihydrospiro[pyrrolidine-3,4′-pyrrolo[1,2-b]pyrazole]-1-carboxamide
(rac)-2′-{6-amino-5-[(pyridin-2-yl)methoxy]pyridin-3-yl}-N-(propan-2-yl)-5′,6′-dihydrospiro[pyrrolidine-3,4′-pyrrolo[1,2-b]pyrazole]-1-carboxamide
(rac)-2′-{6-amino-5-[(pyridin-2-yl)methoxy]pyridin-3-yl}-N-cyclobutyl-5′,6′-dihydrospiro[pyrrolidine-3,4′-pyrrolo[1,2-b]pyrazole]-1-carboxamide
(rac)-2′-{6-amino-5-[(2-chloro-5-fluorophenyl)methoxy]pyridin-3-yl}-N-ethyl-5′,6′-dihydrospiro[pyrrolidine-3,4′-pyrrolo[1,2-b]pyrazole]-1-carboxamide
(rac)-2′-{6-amino-5-[(2-chloro-5-fluorophenyl)methoxy]pyridin-3-yl}-N-(propan-2-yl)-5′,6′-dihydrospiro[pyrrolidine-3,4′-pyrrolo[1,2-b]pyrazole]-1-carboxamide
2′-{6-amino-5-[(1R)-1-(pyridin-2-yl)ethoxy]pyridin-3-yl}-N-(propan-2-yl)-5′,6′-dihydrospiro[pyrrolidine-3,4′-pyrrolo[1,2-b]pyrazole]-1-carboxamide
2′-{6-amino-5-[(1R)-1-(pyridin-2-yl)ethoxy]pyridin-3-yl}-N-ethyl-5′,6′-dihydrospiro[pyrrolidine-3,4′-pyrrolo[1,2-b]pyrazole]-1-carboxamide
2′-{6-amino-5-[(1S)-1-(pyridin-2-yl)ethoxy]pyridin-3-yl}-N-(propan-2-yl)-5′,6′-dihydrospiro[pyrrolidine-3,4′-pyrrolo[1,2-b]pyrazole]-1-carboxamide
2′-{6-amino-5-[(1S)-1-(pyridin-2-yl)ethoxy]pyridin-3-yl}-N-ethyl-5′,6′-dihydrospiro[pyrrolidine-3,4′-pyrrolo[1,2-b]pyrazole]-1-carboxamide
2′-(6-amino-5-{[1-(5-chloro-3-fluoropyridin-2-yl)ethyl]oxy}pyridin-3-yl)-N-ethyl-5′,6′-dihydrospiro[pyrrolidine-3,4′-pyrrolo[1,2-b]pyrazole]-1-carboxamide (diastereomeric mixture)
(rac)-2′-[6-amino-5-(benzyloxy)pyridin-3-yl]-N-cyclobutyl-5′,6′-dihydrospiro[pyrrolidine-3,4′-pyrrolo[1,2-b]pyrazole]-1-carboxamide
2′-{6-amino-5-[(pyridazin-3-yl)methoxy]pyridin-3-yl}-N-ethyl-5′,6′-dihydrospiro[pyrrolidine-3,4′-pyrrolo[1,2-b]pyrazole]-1-carboxamide (stereoisomer 2) 2′-{6-amino-5-[(pyridazin-3-yl)methoxy]pyridin-3-yl}-N-ethyl-5′,6′-dihydrospiro[pyrrolidine-3,4′-pyrrolo[1,2-b]pyrazole]-1-carboxamide (stereoisomer 1)
(3S)-2′-{6-amino-5-[1-(pyrimidin-5-yl)ethoxy]pyridin-3-yl}-N-ethyl-5′,6′-dihydro-1H-spiro[pyrrolidine
3,4′-pyrrolo[1,2-b]pyrazole]-1-carboxamide (isomer 1)
(3S)-2′-{6-amino-5-[1-(pyrimidin-5-yl)ethoxy]pyridin-3-yl}-N-ethyl-5′,6′-dihydro-1H-spiro[pyrrolidine-3,4′-pyrrolo[1,2-b]pyrazole]-1-carboxamide (isomer 2)
(rac)-2′-(2-amino-1′-methyl-1′,2′,3′,6′-tetrahydro[3,4′-bipyridin]-5-yl)-N-ethyl-5′,6′-dihydrospiro[pyrrolidine-3,4′-pyrrolo[1,2-b]pyrazole]-1-carboxamide
2′-{6-amino-5-[(1S)-1-(pyridin-2-yl)ethoxy]pyridin-3-yl}-N-ethyl-5′,6′-dihydrospiro[azetidine-3,4′-pyrrolo[1,2-b]pyrazole]-1-carboxamide
2′-{6-amino-5-[(1S)-1-(pyridin-2-yl)ethoxy]pyridin-3-yl}-N-(propan-2-yl)-5′,6′-dihydrospiro[azetidine-3,4′-pyrrolo[1,2-b]pyrazole]-1-carboxamide
2′-{6-amino-5-[(1S)-1-phenylethoxy]pyridin-3-yl}-N-ethyl-5′,6′-dihydrospiro[azetidine-3,4′-pyrrolo[1,2-b]pyrazole]-1-carboxamide
2′-{6-amino-5-[(1R)-1-phenylethoxy]pyridin-3-yl}-N-ethyl-5′,6′-dihydrospiro[azetidine-3,4′-pyrrolo[1,2-b]pyrazole]-1-carboxamide
(rac)-2-[6-amino-5-(trifluoromethyl)pyridin-3-yl]-N-[2-(3-chloropyridin-4-yl)propan-2-yl]-6,7-dihydrospiro[pyrazolo[5,1-c][1,4]oxazine-4,3′-pyrrolidine]-1′-carboxamide
(rac)-2′-[6-amino-5-(trifluoromethyl)pyridin-3-yl]-N-ethyl-5′,6′-dihydrospiro[pyrrolidine-3,4′-pyrrolo[1,2-b]pyrazole]-1-carboxamide
(rac)-2-[6-amino-5-(trifluoromethyl)pyridin-3-yl]-N-ethyl-6,7-dihydro-5H-spiro[pyrazolo[1,5-a]pyridine-4,3′-pyrrolidine]-1′-carboxamide
(rac)-2′-(6-amino-5-cyanopyridin-3-yl)-N-ethyl-5′,6′-dihydrospiro[pyrrolidine-3,4′-pyrrolo[1,2-b]pyrazole]-1-carboxamide
(rac)-2′-[6-amino-5-(trifluoromethoxy)pyridin-3-yl]-N-ethyl-5′,6′-dihydrospiro[pyrrolidine-3,4′-pyrrolo[1,2-b]pyrazole]-1-carboxamide
(rac)-2′-[6-amino-5-(difluoromethoxy)pyridin-3-yl]-N-ethyl-5′,6′-dihydrospiro[pyrrolidine-3,4′-pyrrolo[1,2-b]pyrazole]-1-carboxamide
(rac)-2′-[6-amino-5-(trifluoroacetyl)pyridin-3-yl]-N-ethyl-5′,6′-dihydrospiro[pyrrolidine-3,4′-pyrrolo[1,2-b]pyrazole]-1-carboxamide
2′-(6-amino-5-{[1-phenylethyl]sulfanyl}pyridin-3-yl)-N-ethyl-5′,6′-dihydrospiro[pyrrolidine-3,4′-pyrrolo[1,2-b]pyrazole]-1-carboxamide (diastereomeric mixture)
(rac)-2′-{6-amino-5-[(cyclohexylmethyl)sulfanyl]pyridin-3-yl}-N-ethyl-5′,6′-dihydrospiro[pyrrolidine-3,4′-pyrrolo[1,2-b]pyrazole]-1-carboxamide
(rac)-2′-[6-amino-5-(benzylsulfanyl)pyridin-3-yl]-N-ethyl-5′,6′-dihydrospiro[pyrrolidine-3,4′-pyrrolo[1,2-b]pyrazole]-1-carboxamide
(rac)-2′-[6-amino-5-(2-hydroxypropan-2-yl)pyridin-3-yl]-N-ethyl-5′,6′-dihydrospiro[pyrrolidine-3,4′-pyrrolo[1,2-b]pyrazole]-1-carboxamide
2′-(6-amino-5-{[1-cyclohexylethyl]sulfanyl}pyridin-3-yl)-N-ethyl-5′,6′-dihydrospiro[pyrrolidine-3,4′-pyrrolo[1,2-b]pyrazole]-1-carboxamide (diastereomeric mixture)
2′-{6-amino-5-[(1R)-1-(pyridin-3-yl)ethoxy]pyridin-3-yl}-N-ethyl-5′,6′-dihydrospiro[pyrrolidine-3,4′-pyrrolo[1,2-b]pyrazole]-1-carboxamide
2′-{6-amino-5-[(1S)-1-(pyridin-3-yl)ethoxy]pyridin-3-yl}-N-ethyl-5′,6′-dihydrospiro[pyrrolidine-3,4′-pyrrolo[1,2-b]pyrazole]-1-carboxamide
(rac)-2′-{6-amino-5-[(cyclopentylmethyl)sulfanyl]pyridin-3-yl}-N-ethyl-5′,6′-dihydrospiro[pyrrolidine-3,4′-pyrrolo[1,2-b]pyrazole]-1-carboxamide
(rac)-2′-(6-amino-5-nitropyridin-3-yl)-N-ethyl-5′,6′-dihydrospiro[pyrrolidine-3,4′-pyrrolo[1,2-b]pyrazole]-1-carboxamide
(rac)-2′-[6-amino-5-(benzyloxy)pyridin-3-yl]-N-ethyl-5′,6′-dihydrospiro[pyrrolidine-3,4′-pyrrolo[1,2-b]pyrazole]-1-carboxamide
(rac)-2′-(6-amino-5-methylpyridin-3-yl)-N-ethyl-5′,6′-dihydrospiro[pyrrolidine-3,4′-pyrrolo[1,2-b]pyrazole]-1-carboxamide
(rac)-2-(6-amino-5-nitropyridin-3-yl)-N-ethyl-6,7-dihydro-5H-spiro[pyrazolo[1,5-a]pyridine-4,3′-pyrrolidine]-1′-carboxamide methyl 2-amino-5-[(rac)-1-(ethylcarbamoyl)-5′,6′-dihydrospiro[pyrrolidine-3,4′-pyrrolo[1,2-b]pyrazol]-2′-yl]pyridine-3-carboxylate
(rac)-2′-(6-amino-5-phenylpyridin-3-yl)-N-ethyl-5′,6′-dihydrospiro[pyrrolidine-3,4′-pyrrolo[1,2-b]pyrazole]-1-carboxamide
(rac)-2′-[6-amino-5-(methylsulfanyl)pyridin-3-yl]-N-ethyl-5′,6′-dihydrospiro[pyrrolidine-3,4′-pyrrolo[1,2-b]pyrazole]-1-carboxamide
(rac)-2′-[6-amino-5-(4-methylphenyl)pyridin-3-yl]-N-ethyl-5′,6′-dihydrospiro[pyrrolidine-3,4′-pyrrolo[1,2-b]pyrazole]-1-carboxamide
(rac)-2′-[6-amino-5-(2-methylphenyl)pyridin-3-yl]-N-ethyl-5′,6′-dihydrospiro[pyrrolidine-3,4′-pyrrolo[1,2-b]pyrazole]-1-carboxamide
(rac)-2′-[6-amino-5-(3-methylphenyl)pyridin-3-yl]-N-ethyl-5′,6′-dihydrospiro[pyrrolidine-3,4′-pyrrolo[1,2-b]pyrazole]-1-carboxamide
(rac)-2′-(6-amino-5-methoxypyridin-3-yl)-N-ethyl-5′,6′-dihydrospiro[pyrrolidine-3,4′-pyrrolo[1,2-b]pyrazole]-1-carboxamide
(rac)-2′-[6-amino-5-(3-fluorophenyl)pyridin-3-yl]-N-ethyl-5′,6′-dihydrospiro[pyrrolidine-3,4′-pyrrolo[1,2-b]pyrazole]-1-carboxamide
(rac)-2′-(2-amino[3,3′-bipyridin]-5-yl)-N-ethyl-5′,6′-dihydrospiro[pyrrolidine-3,4′-pyrrolo[1,2-b]pyrazole]-1-carboxamide
(rac)-2′-[6-amino-5-(2-fluorophenyl)pyridin-3-yl]-N-ethyl-5′,6′-dihydrospiro[pyrrolidine-3,4′-pyrrolo[1,2-b]pyrazole]-1-carboxamide
2′-{6-amino-5-[(1R)-1-(2,6-dichloro-3-fluorophenyl)ethoxy]pyridin-3-yl}-N-ethyl-5′,6′-dihydrospiro[pyrrolidine-3,4′-pyrrolo[1,2-b]pyrazole]-1-carboxamide (diastereomeric mixture)
(rac)-2-[6-amino-5-(2-fluorophenyl)pyridin-3-yl]-N-ethyl-6,7-dihydro-5H-spiro[pyrazolo[1,5-a]pyridine-4,3′-pyrrolidine]-1′-carboxamide
(rac)-2′-[6-amino-5-(2-methoxyphenyl)pyridin-3-yl]-N-ethyl-5′,6′-dihydrospiro[pyrrolidine-3,4′-pyrrolo[1,2-b]pyrazole]-1-carboxamide
(rac)-2′-(2-amino[3,4′-bipyridin]-5-yl)-N-ethyl-5′,6′-dihydrospiro[pyrrolidine-3,4′-pyrrolo[1,2-b]pyrazole]-1-carboxamide
(rac)-2′-{6-amino-5-[3-(methanesulfonyl)phenyl]pyridin-3-yl}-N-ethyl-5′,6′-dihydrospiro[pyrrolidine-3,4′-pyrrolo[1,2-b]pyrazole]-1-carboxamide
(rac)-2′-[6-amino-5-(phenylsulfanyl)pyridin-3-yl]-N-ethyl-5′,6′-dihydrospiro[pyrrolidine-3,4′-pyrrolo[1,2-b]pyrazole]-1-carboxamide
(rac)-2′-{6-amino-5-[4-(methanesulfonyl)phenyl]pyridin-3-yl}-N-ethyl-5′,6′-dihydrospiro[pyrrolidine-3,4′-pyrrolo[1,2-b]pyrazole]-1-carboxamide
(rac)-2′-[5-(4-acetylphenyl)-6-aminopyridin-3-yl]-N-ethyl-5′,6′-dihydrospiro[pyrrolidine-3,4′-pyrrolo[1,2-b]pyrazole]-1-carboxamide
(rac)-2′-{6-amino-5-[4-(methanesulfinyl)phenyl]pyridin-3-yl}-N-ethyl-5′,6′-dihydrospiro[pyrrolidine-3,4′-pyrrolo[1,2-b]pyrazole]-1-carboxamide
(rac)-2′-[6-amino-5-(3-methoxyphenyl)pyridin-3-yl]-N-ethyl-5′,6′-dihydrospiro[pyrrolidine-3,4′-pyrrolo[1,2-b]pyrazole]-1-carboxamide
2′-{6-amino-5-[(1R)-1-(2,6-dichloro-3-fluorophenyl)ethoxy]pyridin-3-yl}-N-ethyl-5′,6′-dihydrospiro[azetidine-3,4′-pyrrolo[1,2-b]pyrazole]-1-carboxamide
(rac)-2′-{6-amino-5-[4-(cyclopropanesulfonyl)phenyl]pyridin-3-yl}-N-ethyl-5′,6′-dihydrospiro[pyrrolidine-3,4′-pyrrolo[1,2-b]pyrazole]-1-carboxamide
(rac)-2′-[6-amino-5-(4-sulfamoylphenyl)pyridin-3-yl]-N-ethyl-5′,6′-dihydrospiro[pyrrolidine-3,4′-pyrrolo[1,2-b]pyrazole]-1-carboxamide
(rac)-2′-{6-amino-5-[4-(methylsulfamoyl)phenyl]pyridin-3-yl}-N-ethyl-5′,6′-dihydrospiro[pyrrolidine-3,4′-pyrrolo[1,2-b]pyrazole]-1-carboxamide
(rac)-2′-{6-amino-5-[4-(dimethylsulfamoyl)phenyl]pyridin-3-yl}-N-ethyl-5′,6′-dihydrospiro[pyrrolidine-3,4′-pyrrolo[1,2-b]pyrazole]-1-carboxamide
(rac)-2′-[6-amino-5-(cyclohexylmethoxy)pyridin-3-yl]-N-ethyl-5′,6′-dihydrospiro[pyrrolidine-3,4′-pyrrolo[1,2-b]pyrazole]-1-carboxamide
(rac)-2′-{6-amino-5-[(pyridin-3-yl)methoxy]pyridin-3-yl}-N-ethyl-5′,6′-dihydrospiro[pyrrolidine-3,4′-pyrrolo[1,2-b]pyrazole]-1-carboxamide
(rac)-2′-{6-amino-5-[(4-fluorophenyl)methoxy]pyridin-3-yl}-N-ethyl-5′,6′-dihydrospiro[pyrrolidine-3,4′-pyrrolo[1,2-b]pyrazole]-1-carboxamide
2′-(6-amino-5-{[1-phenylethyl]oxy}pyridin-3-yl)-N-ethyl-5′,6′-dihydrospiro[pyrrolidine-3,4′-pyrrolo[1,2-b]pyrazole]-1-carboxamide (diastereomeric mixture)
2-{6-amino-5-[(1R)-1-(2,6-dichloro-3-fluorophenyl)ethoxy]pyridin-3-yl}-N-ethyl-6,7-dihydrospiro[pyrazolo[5,1-c][1,4]oxazine-4,3′-pyrrolidine]-1′-carboxamide
(rac)-2′-[6-amino-5-(propan-2-yl)pyridin-3-yl]-N-ethyl-5′,6′-dihydrospiro[pyrrolidine-3,4′-pyrrolo[1,2-b]pyrazole]-1-carboxamide
(rac)-2-[6-amino-5-(methylcarbamoyl)pyridin-3-yl]-N-ethyl-6,7-dihydrospiro[pyrazolo[5,1-c][1,4]oxazine-4,3′-pyrrolidine]-1′-carboxamide
(rac)-2′-[6-amino-5-(methylcarbamoyl)pyridin-3-yl]-N-ethyl-5′,6′-dihydrospiro[pyrrolidine-3,4′-pyrrolo[1,2-b]pyrazole]-1-carboxamide
(rac)-2′-(6-amino-5-cyclopropylpyridin-3-yl)-N-ethyl-5′,6′-dihydrospiro[pyrrolidine-3,4′-pyrrolo[1,2-b]pyrazole]-1-carboxamide
(rac)-2′-[6-amino-5-(methylcarbamoyl)pyridin-3-yl]-N-(1-phenylcyclobutyl)-5′,6′-dihydrospiro[pyrrolidine-3,4′-pyrrolo[1,2-b]pyrazole]-1-carboxamide
(rac)-2′-[6-amino-5-(dimethylcarbamoyl)pyridin-3-yl]-N-(1-phenylcyclobutyl)-5′,6′-dihydrospiro[pyrrolidine-3,4′-pyrrolo[1,2-b]pyrazole]-1-carboxamide
2′-{6-amino-5-[(1R)-1-(pyridin-4-yl)ethoxy]pyridin-3-yl}-N-ethyl-5′,6′-dihydrospiro[pyrrolidine-3,4′-pyrrolo[1,2-b]pyrazole]-1-carboxamide
2′-{6-amino-5-[(1S)-1-(pyridin-4-yl)ethoxy]pyridin-3-yl}-N-ethyl-5′,6′-dihydrospiro[pyrrolidine-3,4′-pyrrolo[1,2-b]pyrazole]-1-carboxamide
(rac)-2′-[6-amino-5-(1-methyl-1H-pyrazol-3-yl)pyridin-3-yl]-N-ethyl-5′,6′-dihydrospiro[pyrrolidine-3,4′-pyrrolo[1,2-b]pyrazole]-1-carboxamide
(rac)-2′-[6-amino-5-(1,2-thiazol-5-yl)pyridin-3-yl]-N-ethyl-5′,6′-dihydrospiro[pyrrolidine-3,4′-pyrrolo[1,2-b]pyrazole]-1-carboxamide
(rac)-2′-[6-amino-5-(1,5-dimethyl-1H-pyrazol-4-yl)pyridin-3-yl]-N-ethyl-5′,6′-dihydrospiro[pyrrolidine-3,4′-pyrrolo[1,2-b]pyrazole]-1-carboxamide
(rac)-2′-{6-amino-5-[(trifluoromethyl)sulfanyl]pyridin-3-yl}-N-ethyl-5′,6′-dihydrospiro[pyrrolidine-3,4′-pyrrolo[1,2-b]pyrazole]-1-carboxamide
5-[1-methyl-5′,6′-dihydrospiro[pyrrolidine-3,4′-pyrrolo[1,2-b]pyrazol]-2′-yl]-3-(trifluoromethyl)pyridin-2-amine (stereoisomer 1)
5-[1-methyl-5′,6′-dihydrospiro[pyrrolidine-3,4′-pyrrolo[1,2-b]pyrazol]-2′-yl]-3-(trifluoromethyl)pyridin-2-amine (stereoisomer 2)
(3R)-2′-{6-amino-5-[(1R)-1-(2-chlorophenyl)ethoxy]pyridin-3-yl}-N-ethyl-5′,6′-dihydrospiro[pyrrolidine-3,4′-pyrrolo[1,2-b]pyrazole]-1-carboxamide
2′-{6-amino-5-[(1R)-1-(2-chlorophenyl)ethoxy]pyridin-3-yl}-N-ethyl-5′,6′-dihydrospiro[azetidine-3,4′-pyrrolo[1,2-b]pyrazole]-1-carboxamide
2′-{6-amino-5-[(1S)-1-(2-chlorophenyl)ethoxy]pyridin-3-yl}-N-ethyl-5′,6′-dihydrospiro[azetidine-3,4′-pyrrolo[1,2-b]pyrazole]-1-carboxamide
(3′R)-2-{6-amino-5-[(1R)-1-(2,6-dichloro-3-fluorophenyl)ethoxy]pyridin-3-yl}-N-ethyl-6,7-dihydrospiro[pyrazolo[5,1-c][1,4]oxazine-4,3′-pyrrolidine]-1′-carboxamide
(3R)-2′-{6-amino-5-[(1R)-1-(pyridin-2-yl)ethoxy]pyridin-3-yl}-N-ethyl-5′,6′-dihydrospiro[pyrrolidine-3,4′-pyrrolo[1,2-b]pyrazole]-1-carboxamide
2′-{6-amino-5-[(1R)-1-(pyridin-3-yl)ethoxy]pyridin-3-yl}-N-ethyl-5′,6′-dihydrospiro[azetidine-3,4′-pyrrolo[1,2-b]pyrazole]-1-carboxamide
(3R)-2′-{6-amino-5-[1-(pyrimidin-5-yl)ethoxy]pyridin-3-yl}-N-ethyl-5′,6′-dihydro-1H-spiro[pyrrolidine-3,4′-pyrrolo[1,2-b]pyrazole]-1-carboxamide (isomer 1)
((3R)-2′-{6-amino-5-[1-(pyrimidin-5-yl)ethoxy]pyridin-3-yl}-N-ethyl-5′,6′-dihydro-1H-spiro[pyrrolidine-3,4′-pyrrolo[1,2-b]pyrazole]-1-carboxamide (isomer 2)
(rac)-2′-[6-amino-5-(difluoromethyl)pyridin-3-yl]-N-ethyl-5′,6′-dihydrospiro[pyrrolidine-3,4′-pyrrolo[1,2-b]pyrazole]-1-carboxamide
2′-{6-amino-5-[(1S)-1-(2-chlorophenyl)ethoxy]pyridin-3-yl}-N-ethyl-5′,6′-dihydrospiro[pyrrolidine-3,4′-pyrrolo[1,2-b]pyrazole]-1-carboxamide
2′-{6-amino-5-[(1R)-1-(2-chlorophenyl)ethoxy]pyridin-3-yl}-N-ethyl-5′,6′-dihydrospiro[pyrrolidine-3,4′-pyrrolo[1,2-b]pyrazole]-1-carboxamide
(rac)-2′-{6-amino-5-[(3-methylphenyl)methoxy]pyridin-3-yl}-N-ethyl-5′,6′-dihydrospiro[pyrrolidine-3,4′-pyrrolo[1,2-b]pyrazole]-1-carboxamide
(rac)-2′-{6-amino-5-[(1-methyl-1H-pyrazol-5-yl)methoxy]pyridin-3-yl}-N-ethyl-5′,6′-dihydrospiro[pyrrolidine-3,4′-pyrrolo[1,2-b]pyrazole]-1-carboxamide
(rac)-2′-{6-amino-5-[(1-methyl-1H-pyrazol-4-yl)methoxy]pyridin-3-yl}-N-ethyl-5′,6′-dihydrospiro[pyrrolidine-3,4′-pyrrolo[1,2-b]pyrazole]-1-carboxamide
(rac)-2′-{6-amino-5-[(1,5-dimethyl-1H-pyrazol-3-yl)methoxy]pyridin-3-yl}-N-ethyl-5′,6′-dihydrospiro[pyrrolidine-3,4′-pyrrolo[1,2-b]pyrazole]-1-carboxamide
(rac)-2′-{6-amino-5-[(4-methylphenyl)methoxy]pyridin-3-yl}-N-ethyl-5′,6′-dihydrospiro[pyrrolidine-3,4′-pyrrolo[1,2-b]pyrazole]-1-carboxamide
2′-{6-amino-5-[(1R)-1-(3-methylphenyl)ethoxy]pyridin-3-yl}-N-ethyl-5′,6′-dihydrospiro[pyrrolidine-3,4′-pyrrolo[1,2-b]pyrazole]-1-carboxamide
(rac)-2′-{6-amino-5-[(propan-2-yl)oxy]pyridin-3-yl}-N-ethyl-5′,6′-dihydrospiro[pyrrolidine-3,4′-pyrrolo[1,2-b]pyrazole]-1-carboxamide
(3R)-2′-{6-amino-5-[(1S)-1-(1-methyl-1H-pyrazol-3-yl)ethoxy]pyridin-3-yl}-N-ethyl-5′,6′-dihydrospiro[pyrrolidine-3,4′-pyrrolo[1,2-b]pyrazole]-1-carboxamide
(3R)-2′-{6-amino-5-[(1R)-1-(1-methyl-1H-pyrazol-3-yl)ethoxy]pyridin-3-yl}-N-ethyl-5′,6′-dihydrospiro[pyrrolidine-3,4′-pyrrolo[1,2-b]pyrazole]-1-carboxamide
(3R)-2′-(6-amino-5-{[1-(3,5-difluoropyridin-2-yl)ethyl]oxy}pyridin-3-yl)-N-ethyl-5′,6′-dihydrospiro[pyrrolidine-3,4′-pyrrolo[1,2-b]pyrazole]-1-carboxamide (stereoisomer 1)
(3R)-2′-(6-amino-5-{[1-(3,5-difluoropyridin-2-yl)ethyl]oxy}pyridin-3-yl)-N-ethyl-5′,6′-dihydrospiro[pyrrolidine-3,4′-pyrrolo[1,2-b]pyrazole]-1-carboxamide (stereoisomer 2)
(3R)-2′-{6-amino-5-[(1R)-1-(2-fluorophenyl)ethoxy]pyridin-3-yl}-N-ethyl-5′,6′-dihydrospiro[pyrrolidine-3,4′-pyrrolo[1,2-b]pyrazole]-1-carboxamide
(3R)-2′-{6-amino-5-[(1S)-1-(2-fluorophenyl)ethoxy]pyridin-3-yl}-N-ethyl-5′,6′-dihydrospiro[pyrrolidine-3,4′-pyrrolo[1,2-b]pyrazole]-1-carboxamide
(rac)-2′-(5-acetyl-6-aminopyridin-3-yl)-N-(1-phenylcyclobutyl)-5′,6′-dihydrospiro[pyrrolidine-3,4′-pyrrolo[1,2-b]pyrazole]-1-carboxamide
(3R)-2′-[6-amino-5-(trifluoromethoxy)pyridin-3-yl]-N-[2-(pyridin-2-yl)propan-2-yl]-5′,6′-dihydrospiro[pyrrolidine-3,4′-pyrrolo[1,2-b]pyrazole]-1-carboxamide
(3′R)-2-[6-amino-5-(trifluoromethyl)pyridin-3-yl]-N-[2-(pyridin-2-yl)propan-2-yl]-6,7-dihydrospiro[pyrazolo[5,1-c][1,4]oxazine-4,3′-pyrrolidine]-1′-carboxamide
(3R)-2′-(6-amino-5-cyanopyridin-3-yl)-N-[2-(pyridin-2-yl)propan-2-yl]-5′,6′-dihydrospiro[pyrrolidine-3,4′-pyrrolo[1,2-b]pyrazole]-1-carboxamide
(3′R)-2-(6-amino-5-cyanopyridin-3-yl)-N-[2-(pyridin-2-yl)propan-2-yl]-6,7-dihydrospiro[pyrazolo[5,1-c][1,4]oxazine-4,3′-pyrrolidine]-1′-carboxamide
(rac)-2′-[6-amino-5-(phenylcarbamoyl)pyridin-3-yl]-N-ethyl-5′,6′-dihydrospiro[pyrrolidine-3,4′-pyrrolo[1,2-b]pyrazole]-1-carboxamide
(rac)-2′-[6-amino-5-(cyclohexylcarbamoyl)pyridin-3-yl]-N-ethyl-5′,6′-dihydrospiro[pyrrolidine-3,4′-pyrrolo[1,2-b]pyrazole]-1-carboxamide
(rac)-2′-[6-amino-5-(diethylcarbamoyl)pyridin-3-yl]-N-ethyl-5′,6′-dihydrospiro[pyrrolidine-3,4′-pyrrolo[1,2-b]pyrazole]-1-carboxamide
(rac)-2′-(5-acetyl-6-aminopyridin-3-yl)-N-ethyl-5′,6′-dihydrospiro[pyrrolidine-3,4′-pyrrolo[1,2-b]pyrazole]-1-carboxamide
5-{1-[1-(1H-imidazol-2-yl)ethyl]-5′,6′-dihydrospiro[pyrrolidine-3,4′-pyrrolo[1,2-b]pyrazol]-2′-yl}-3-(trifluoromethyl)pyridin-2-amine
5-{1-[1-(1H-pyrazol-3-yl)ethyl]-5′,6′-dihydrospiro[pyrrolidine-3,4′-pyrrolo[1,2-b]pyrazol]-2′-yl}-3-(trifluoromethyl)pyridin-2-amine
5-{(3R)-1-[1-(1H-pyrazol-3-yl)ethyl]-5′,6′-dihydrospiro[pyrrolidine-3,4′-pyrrolo[1,2-b]pyrazol]-2′-yl}-3-(trifluoromethyl)pyridin-2-amine
5-{(3R)-1-[1-(1H-imidazol-2-yl)ethyl]-5′,6′-dihydrospiro[pyrrolidine-3,4′-pyrrolo[1,2-b]pyrazol]-2′-yl}-3-(trifluoromethyl)pyridin-2-amine
5-{(3R)-1-[1-(1H-imidazol-5-yl)ethyl]-5′,6′-dihydrospiro[pyrrolidine-3,4′-pyrrolo[1,2-b]pyrazol]-2′-yl}-3-(trifluoromethyl)pyridin-2-amine
5-{(3R)-1-[1-(1-methyl-1H-pyrazol-3-yl)ethyl]-5′,6′-dihydrospiro[pyrrolidine-3,4′-pyrrolo[1,2-b]pyrazol]-2′-yl}-3-(trifluoromethyl)pyridin-2-amine
5-{(3R)-1-[1-(1H-pyrazol-4-yl)ethyl]-5′,6′-dihydrospiro[pyrrolidine-3,4′-pyrrolo[1,2-b]pyrazol]-2′-yl}-3-(trifluoromethyl)pyridin-2-amine
5-{(3R)-1-[1-(4-methyl-1H-imidazol-2-yl)ethyl]-5′,6′-dihydrospiro[pyrrolidine-3,4′-pyrrolo[1,2-b]pyrazol]-2′-yl}-3-(trifluoromethyl)pyridin-2-amine
5-{(3R)-1-[1-(3-methyl-1H-pyrazol-5′,6′-dihydrospiro[pyrrolidine-3,4′-pyrrolo[1,2-b]pyrazol]-2′-yl}-3-(trifluoromethyl)pyridin-2-amine
5-{(3R)-1-[1-(4-methyl-1H-pyrazol-5-yl)ethyl]-5′,6′-dihydrospiro[pyrrolidine-3,4′-pyrrolo[1,2-b]pyrazol]-2′-yl}-3-(trifluoromethyl)pyridin-2-amine
5-[(3S)-1-(pyridin-3-ylmethyl)-5′,6′-dihydrospiro[pyrrolidine-3,4′-pyrrolo[1,2-b]pyrazol]-2′-yl]-3-(trifluoromethyl)pyridin-2-amine
5-[(3R)-1-(pyridin-3-ylmethyl)-5′,6′-dihydrospiro[pyrrolidine-3,4′-pyrrolo[1,2-b]pyrazol]-2′-yl]-3-(trifluoromethyl)pyridin-2-amine
5-{(rac)-1-[(1H-pyrazol-3-yl)methyl]-5′,6′-dihydrospiro[pyrrolidine-3,4′-pyrrolo[1,2-b]pyrazol]-2′-yl}-3-(trifluoromethyl)pyridin-2-amine
5-[(3S)-1-(1H-imidazol-2-ylmethyl)-5′,6′-dihydrospiro[pyrrolidine-3,4′-pyrrolo[1,2-b]pyrazol]-2′-yl]-3-(trifluoromethyl)pyridin-2-amine
5-[(3R)-1-(1H-imidazol-2-ylmethyl)-5′,6′-dihydrospiro[pyrrolidine-3,4′-pyrrolo[1,2-b]pyrazol]-2′-yl]-3-(trifluoromethyl)pyridin-2-amine
5-{(rac)-1-[(4-methyl-1H-pyrazol-3-yl)methyl]-5′,6′-dihydrospiro[pyrrolidine-3,4′-pyrrolo[1,2-b]pyrazol]-2′-yl}-3-(trifluoromethyl)pyridin-2-amine
5-{(rac)-1-[(1H-pyrazol-4-yl)methyl]-5′,6′-dihydrospiro[pyrrolidine-3,4′-pyrrolo[1,2-b]pyrazol]-2′-yl}-3-(trifluoromethyl)pyridin-2-amine
5-{(rac)-1-[(1-methyl-1H-pyrazol-4-yl)methyl]-5′,6′-dihydrospiro[pyrrolidine-3,4′-pyrrolo[1,2-b]pyrazol]-2′-yl}-3-(trifluoromethyl)pyridin-2-amine
5-{(rac)-1-[(1H-imidazol-4-yl)methyl]-5′,6′-dihydrospiro[pyrrolidine-3,4′-pyrrolo[1,2-b]pyrazol]-2′-yl}-3-(trifluoromethyl)pyridin-2-amine
5-{(rac)-1-[(1-methyl-1H-imidazol-4-yl)methyl]-5′,6′-dihydrospiro[pyrrolidine-3,4′-pyrrolo[1,2-b]pyrazol]-2′-yl}-3-(trifluoromethyl)pyridin-2-amine
5-{(rac)-1-[(4-chloro-1H-pyrazol-5-yl)methyl]-5′,6′-dihydrospiro[pyrrolidine-3,4′-pyrrolo[1,2-b]pyrazol]-2′-yl}-3-(trifluoromethyl)pyridin-2-amine
3-[(2-fluorophenyl)methoxy]-5-{(rac)-1-[(1H-imidazol-2-yl)methyl]-5′,6′-dihydrospiro[pyrrolidine-3,4′-pyrrolo[1,2-b]pyrazol]-2′-yl}pyridin-2-amine
3-[(3-fluorophenyl)methoxy]-5-{(rac)-1-[(1H-imidazol-2-yl)methyl]-5′,6′-dihydrospiro[pyrrolidine-3,4′-pyrrolo[1,2-b]pyrazol]-2′-yl}pyridin-2-amine
5-{(rac)-1-[(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine
5-{1-[(1H-imidazol-2-yl)methyl]-5′,6′-dihydrospiro[azetidine-3,4′-pyrrolo[1,2-b]pyrazol]-2′-yl}-3-(trifluoromethyl)pyridin-2-amine
5-{(rac)-1′-[(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine
2-amino-5-{1-[(1H-imidazol-2-yl)methyl]-5′,6′-dihydrospiro[azetidine-3,4′-pyrrolo[1,2-b]pyrazol]-2′-yl}pyridine-3-carbonitrile
5-{(rac)-1-[(1-methyl-1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine
5-{(rac)-1-[(1-methyl-1H-pyrazol-3-yl)methyl]-5′,6′-dihydrospiro[pyrrolidine-3,4′-pyrrolo[1,2-b]pyrazol]-2′-yl}-3-(trifluoromethyl)pyridin-2-amine
2-amino-5-{(rac)-1-[(1H-imidazol-2-yl)methyl]-5′,6′-dihydrospiro[pyrrolidine-3,4′-pyrrolo[1,2-b]pyrazol]-2′-yl}pyridine-3-carbonitrile
5-{1-[(1-methyl-1H-imidazol-2-yl)methyl]-5′,6′-dihydrospiro[azetidine-3,4′-pyrrolo[1,2-b]pyrazol]-2′-yl}-3-(trifluoromethyl)pyridin-2-amine
5-{(rac)-1-[(5-methyl-1H-pyrazol-3-yl)methyl]-5′,6′-dihydrospiro[pyrrolidine-3,4′-pyrrolo[1,2-b]pyrazol]-2′-yl}-3-(trifluoromethyl)pyridin-2-amine
5-{(rac)-1-[(2,4-dimethyl-1H-imidazol-5-yl)methyl]-5′,6′-dihydrospiro[pyrrolidine-3,4′-pyrrolo[1,2-b]pyrazol]-2′-yl}-3-(trifluoromethyl)pyridin-2-amine
5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine
5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (stereoisomer 1)
5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (stereoisomer 2)
5-{(3R)-1-[(1-(1H-imidazol-2-yl)propyl]-5′,6′-dihydrospiro[pyrrolidine-3,4′-pyrrolo[1,2-b]pyrazol]-2′-yl}-3-(trifluoromethyl)pyridin-2-amine
5-{(3R)-1-[1-(1H-imidazol-2-yl)propyl]-5′,6′-dihydrospiro[pyrrolidine-3,4′-pyrrolo[1,2-b]pyrazol]-2′-yl}-3-(trifluoromethyl)pyridin-2-amine (stereoisomer 1)
5-{(3R)-1-[1-(1H-imidazol-2-yl)propyl]-5′,6′-dihydrospiro[pyrrolidine-3,4′-pyrrolo[1,2-b]pyrazol]-2′-yl}-3-(trifluoromethyl)pyridin-2-amine (stereoisomer 2)
5-{(3R)-1-[1-(1-methyl-1H-imidazol-2-yl)ethyl]-5′,6′-dihydrospiro[pyrrolidine-3,4′-pyrrolo[1,2-b]pyrazol]-2′-yl}-3-(trifluoromethyl)pyridin-2-amine
3-(trifluoromethyl)-5-[(3R)-1-{[4-(trifluoromethyl)-1H-imidazol-2-yl]methyl}-5′,6′-dihydrospiro[pyrrolidine-3,4′-pyrrolo[1,2-b]pyrazol]-2′-yl]pyridin-2-amine
2-amino-5-[(rac)-1-(ethylcarbamoyl)-5′,6′-dihydrospiro[pyrrolidine-3,4′-pyrrolo[1,2-b]pyrazol]-2′-yl]pyridine-3-carboxylic acid
5-[(3S)-1-(pyridin-2-ylmethyl)-5′,6′-dihydrospiro[pyrrolidine-3,4′-pyrrolo[1,2-b]pyrazol]-2′-yl]-3-(trifluoromethyl)pyridin-2-amine
5-[(3R)-1-(pyridin-2-ylmethyl)-5′,6′-dihydrospiro[pyrrolidine-3,4′-pyrrolo[1,2-b]pyrazol]-2′-yl]-3-(trifluoromethyl)pyridin-2-amine
5-[(3S)-1-(pyridin-4-ylmethyl)-5′,6′-dihydrospiro[pyrrolidine-3,4′-pyrrolo[1,2-b]pyrazol]-2′-yl]-3-(trifluoromethyl)pyridin-2-amine
5-[(3R)-1-(pyridin-4-ylmethyl)-5′,6′-dihydrospiro[pyrrolidine-3,4′-pyrrolo[1,2-b]pyrazol]-2′-yl]-3-(trifluoromethyl)pyridin-2-amine
5-{(3R)-1-[3-(dimethylamino)propyl]-5′,6′-dihydrospiro[pyrrolidine-3,4′-pyrrolo[1,2-b]pyrazol]-2′-yl}-3-(trifluoromethyl)pyridin-2-amine
5-{(3R)-1-[2-(dimethylamino)ethyl]-5′,6′-dihydrospiro[pyrrolidine-3,4′-pyrrolo[1,2-b]pyrazol]-2′-yl}-3-(trifluoromethyl)pyridin-2-amine
2-amino-1-{2′-[6-amino-5-(trifluoromethyl)pyridin-3-yl]-5′,6′-dihydrospiro[pyrrolidine-3,4′-pyrrolo[1,2-b]pyrazol]-1-yl}ethan-1-one (stereoisomer 2)
2-amino-1-{2′-[6-amino-5-(trifluoromethyl)pyridin-3-yl]-5′,6′-dihydrospiro[pyrrolidine-3,4′-pyrrolo[1,2-b]pyrazol]-1-yl}ethan-1-one (stereoisomer 1)
2-amino-1-{2-[6-amino-5-(trifluoromethyl)pyridin-3-yl]-6,7-dihydrospiro[pyrazolo[5,1-c][1,4]oxazine-4,3′-pyrrolidin]-1′-yl}ethan-1-one (stereoisomer 2)
2-amino-1-{2-[6-amino-5-(trifluoromethyl)pyridin-3-yl]-6,7-dihydrospiro[pyrazolo[5,1-c][1,4]oxazine-4,3′-pyrrolidin]-1′-yl}ethan-1-one (stereoisomer 1)
2-amino-1-{2′-[6-amino-5-(trifluoromethyl)pyridin-3-yl]-5′,6′-dihydrospiro[azetidine-3,4′-pyrrolo[1,2-b]pyrazol]-1-yl}ethan-1-one
(2S)-2-amino-1-{2′-[6-amino-5-(trifluoromethyl)pyridin-3-yl]-5′,6′-dihydrospiro[azetidine-3,4′-pyrrolo[1,2-b]pyrazol]-1-yl}propan-1-one
(2R)-2-amino-1-{2′-[6-amino-5-(trifluoromethyl) pyridin-3-yl]-5′,6′-dihydrospiro[azetidine-3,4′-pyrrolo[1,2-b]pyrazol]-1-yl}propan-1-one
(rac)-2′-[6-amino-5-(1-methyl piperidin-4-yl)pyridin-3-yl]-N-ethyl-5′,6′-dihydrospiro[pyrrolidine-3,4′-pyrrolo[1,2-b]pyrazole]-1-carboxamide
5-{(rac)-1-[(5-methyl-1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine
2′-{6-amino-5-[(1R)-1-(2,6-dichloro-3-fluorophenyl)ethoxy]pyridin-3-yl}-N-ethyl-5′,6′-dihydrospiro[pyrrolidine-3,4′-pyrrolo[1,2-b]pyrazole]-1-carboxamide (isomer 1)
2′-{6-amino-5-[(1R)-1-(2,6-dichloro-3-fluorophenyl)ethoxy]pyridin-3-yl}-N-ethyl-5′,6′-dihydrospiro[pyrrolidine-3,4′-pyrrolo[1,2-b]pyrazole]-1-carboxamide (isomer 2)
5-{(3′R)-1′-[(1S)-1-(1H-imidazol-2-yl)propyl]-6,7-dihydrospiro[pyrazolo[5,1-c][1,4]oxazine-4,3′-pyrrolidin]-2-yl}-3-(trifluoromethyl)pyridin-2-amine
5-{(3′R)-1′-[(1R)-1-(1H-imidazol-2-yl)propyl]-6,7-dihydrospiro[pyrazolo[5,1-c][1,4]oxazine-4,3′-pyrrolidin]-2-yl}-3-(trifluoromethyl)pyridin-2-amine
5-{(3′R)-1′-[(S)-cyclopropyl(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine
5-{(3′R)-1′-[(R)-cyclopropyl(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine
5-{(3R)-1-[(S)-cyclopropyl(1H-imidazol-2-yl)methyl]-5′,6′-dihydrospiro[pyrrolidine-3,4′-pyrrolo[1,2-b]pyrazol]-2′-yl}-3-(trifluoromethoxy)pyridin-2-amine
5-{(3R)-1-[(R)-cyclopropyl(1H-imidazol-2-yl)methyl]-5′,6′-dihydrospiro[pyrrolidine-3,4′-pyrrolo[1,2-b]pyrazol]-2′-yl}-3-(trifluoromethoxy)pyridin-2-amine
5-{(3R)-1-[(1S)-1-(1H-imidazol-2-yl)propyl]-5′,6′-dihydrospiro[pyrrolidine-3,4′-pyrrolo[1,2-b]pyrazol]2′-yl}-3-(trifluoromethoxy)pyridin-2-amin-2-amine
5-{(3R)-1-[(1R)-1-(1H-imidazol-2-yl)propyl]-5′,6′-dihydrospiro[pyrrolidine-3,4′-pyrrolo[1,2-b]pyrazol
2′-yl}-3-(trifluoromethoxy)pyridin-2-amine
5-{(3R)-1-[(R)-cyclopropyl(1H-imidazol-2-yl)methyl]-5′,6′-dihydrospiro[pyrrolidine-3,4′-pyrrolo[1,2-b]pyrazol]-2′-yl}-3-(difluoromethoxy)pyridin-2-amine
5-{(3R)-1-[(S)-cyclopropyl(1H-imidazol-2-yl)methyl]-5′,6′-dihydrospiro[pyrrolidine-3,4′-pyrrolo[1,2-b]pyrazol]-2′-yl}-3-(difluoromethoxy)pyridin-2-amine
3-(difluoromethoxy)-5-{(3R)-1-[(1R)-1-(1H-imidazol-2-yl)propyl]-5′,6′-dihydrospiro[pyrrolidine-3,4′-pyrrolo[1,2-b]pyrazol]-2′-yl}pyridin-2-amine
3-(difluoromethoxy)-5-{1(3R)-1-[(1S)-1-(1H-imidazol-2-yl)propyl]-5′,6′-dihydrospiro[pyrrolidine-3,4′-pyrrolo[1,2-b]pyrazol]-2′-yl}pyridin-2-amine
2-amino-5-{(3R)-1-[(R)-cyclopropyl(1H-imidazol-2-yl)methyl]-5′,6′-dihydrospiro[pyrrolidine-3,4′-pyrrolo[1,2-b]pyrazol]-2′-yl}nicotinonitrile
2-amino-5-{(3R)-1-[(S)-cyclopropyl(1H-imidazol-2-yl)methyl]-5′,6′-dihydrospiro[pyrrolidine-3,4′-pyrrolo[1,2-b]pyrazol]-2′-yl}nicotinonitrile
2-amino-5-{(3R)-1-[(1R)-1-(1H-imidazol-2-yl)propyl]-5′,6′-dihydrospiro[pyrrolidine-3,4′-pyrrolo[1,2-b]pyrazol]-2′-yl}nicotinonitrile
2-amino-5-{(3R)-1-[(1S)-1-(1H-imidazol-2-yl)propyl]-5′,6′-dihydrospiro[pyrrolidine-3,4′-pyrrolo[1,2-b]pyrazol]-2′-yl}nicotinonitrile
(3R)-2′-(6-amino-5-{[1-(6-methyl pyridin-2-yl)ethyl]oxy}pyridin-3-yl)-N-ethyl-5′,6′-dihydrospiro[pyrrolidine-3,4′-pyrrolo[1,2-b]pyrazole]-1-carboxamide (diastereomer 1)
(3R)-2′-(6-amino-5-{[1-(6-methylpyridin-2-yl)ethyl]oxy}pyridin-3-yl)-N-ethyl-5′,6′-dihydrospiro[pyrrolidine-3,4′-pyrrolo[1,2-b]pyrazole]-1-carboxamide (diastereomer 2)
(3R)-2′-(6-amino-5-{[1-(5-methyl pyridin-2-yl)ethyl]oxy}pyridin-3-yl)-N-ethyl-5′,6′-dihydrospiro[pyrrolidine-3,4′-pyrrolo[1,2-b]pyrazole]-1-carboxamide (diastereomer 1)
(3R)-2′-(6-amino-5-{[1-(5-methylpyridin-2-yl)ethyl]oxy}pyridin-3-yl)-N-ethyl-5′,6′-dihydrospiro[pyrrolidine-3,4′-pyrrolo[1,2-b]pyrazole]-1-carboxamide (diastereomer 2)
(3R)-2′-(6-amino-5-{[1-(4-methylpyridin-2-yl)ethyl]oxy}pyridin-3-yl)-N-ethyl-5′,6′-dihydrospiro[pyrrolidine-3,4′-pyrrolo[1,2-b]pyrazole]-1-carboxamide (diastereomer 1)
(3R)-2′-(6-amino-5-{[1-(4-methylpyridin-2-yl)ethyl]oxy}pyridin-3-yl)-N-ethyl-5′,6′-dihydrospiro[pyrrolidine-3,4′-pyrrolo[1,2-b]pyrazole]-1-carboxamide (diastereomer 2)
(3R)-2′-(6-amino-5-{[1-(3-methylpyridin-2-yl)ethyl]oxy}pyridin-3-yl)-N-ethyl-5′,6′-dihydrospiro[pyrrolidine-3,4′-pyrrolo[1,2-b]pyrazole]-1-carboxamide (diastereomer 1)
(3R)-2′-(6-amino-5-{[1-(3-methylpyridin-2-yl)ethyl]oxy}pyridin-3-yl)-N-ethyl-5′,6′-dihydrospiro[pyrrolidine-3,4′-pyrrolo[1,2-b]pyrazole]-1-carboxamide (diastereomer 2)
(3R)-2′-(6-amino-5-{[1-(1,4-dimethyl-1H-pyrazol-5-yl)ethyl]oxy}pyridin-3-yl)-N-ethyl-5′,6′-dihydrospiro[pyrrolidine-3,4′-pyrrolo[1,2-b]pyrazole]-1-carboxamide (diastereomer 1)
(3R)-2′-(6-amino-5-{[1-(1,4-dimethyl-1H-pyrazol-5-yl)ethyl]oxy}pyridin-3-yl)-N-ethyl-5′,6′-dihydrospiro[pyrrolidine-3,4′-pyrrolo[1,2-b]pyrazole]-1-carboxamide (diastereomer 2)
(3R)-2′-(6-amino-5-{[−1-(1,4-dimethyl-1H-pyrazol-5-yl)ethyl]oxy}pyridin-3-yl)-N-(propan-2-yl)-5′,6′-dihydrospiro[pyrrolidine-3,4′-pyrrolo[1,2-b]pyrazole]-1-carboxamide (diastereomer 1)
(3R)-2′-(6-amino-5-{[1-(1,4-dimethyl-1H-pyrazol-5-yl)ethyl]oxy}pyridin-3-yl)-N-(propan-2-yl)-5′,6′-dihydrospiro[pyrrolidine-3,4′-pyrrolo[1,2-b]pyrazole]-1-carboxamide (diastereomer 2)
(3R)-2′-{6-amino-5-[(1S)-1-(2,6-difluorophenyl)ethoxy]pyridin-3-yl}-N-ethyl-5′,6′-dihydrospiro[pyrrolidine-3,4′-pyrrolo[1,2-b]pyrazole]-1-carboxamide
(3R)-2′-{6-amino-5-[(1R)-1-(5-fluoropyridin-2-yl)ethoxy]pyridin-3-yl}-N-ethyl-5′,6′-dihydrospiro[pyrrolidine-3,4′-pyrrolo[1,2-b]pyrazole]-1-carboxamide
(3R)-2′-{6-amino-5-[(1R)-1-(3-fluoropyridin-2-yl)ethoxy]pyridin-3-yl}-N-ethyl-5′,6′-dihydrospiro[pyrrolidine-3,4′-pyrrolo[1,2-b]pyrazole]-1-carboxamide
(3R)-2′-{6-amino-5-[(1R)-1-(2,6-difluorophenyl)ethoxy]pyridin-3-yl}-N-ethyl-5′,6′-dihydrospiro[pyrrolidine-3,4′-pyrrolo[1,2-b]pyrazole]-1-carboxamide
(3R)-2′-{6-amino-5-[1-(2-cyanophenyl)ethoxy]pyridin-3-yl}-N-ethyl-5′,6′-dihydro-1H-spiro[pyrrolidine-3,4′-pyrrolo[1,2-b]pyrazole]-1-carboxamide (diastereomer 1 and diastereomer 2)
(3R)-2′-{6-amino-5-[1-(2-cyanophenyl)ethoxy]pyridin-3-yl}-N-ethyl-5′,6′-dihydro-1H-spiro[pyrrolidine-3,4′-pyrrolo[1,2-b]pyrazole]-1-carboxamide (diastereomer 1)
(3R)-2′-{6-amino-5-[1-(2-cyanophenyl)ethoxy]pyridin-3-yl}-N-ethyl-5′,6′-dihydro-1H-spiro[pyrrolidine-3,4′-pyrrolo[1,2-b]pyrazole]-1-carboxamide (diastereomer 2)
(3′R)-2-[6-amino-5-(trifluoromethyl)pyridin-3-yl]-N-[1-(1-ethyl-1H-pyrazol-3-yl)ethyl]-6,7-dihydro-1′H-spiro[pyrazolo[5,1-c][1,4]oxazine-4,3′-pyrrolidine]-1′-carboxamide (diastereomer 1 and diastereomer 2)
(3′R)-2-[6-amino-5-(trifluoromethyl)pyridin-3-yl]-N-[(1-methyl-1H-pyrazol-5-yl)methyl]-6,7-dihydrospiro[pyrazolo[5,1-c][1,4]oxazine-4,3′-pyrrolidine]-1′-carboxamide
(3′R)-2-[6-amino-5-(trifluoromethyl)pyridin-3-yl]-N-[(1-methyl-1H-pyrazol-4-yl)methyl]-6,7-dihydrospiro[pyrazolo[5,1-c][1,4]oxazine-4,3′-pyrrolidine]-1′-carboxamide
(3′R)-2-[6-amino-5-(trifluoromethyl)pyridin-3-yl]-N-[1-(1-methyl-1H-pyrazol-5-yl)ethyl]-6,7-dihydro-1′H-spiro[pyrazolo[5,1-c][1,4]oxazine-4,3′-pyrrolidine]-1′-carboxamide (diastereomer 1 and diastereomer 2)
(3′R)-2-[6-amino-5-(trifluoromethyl)pyridin-3-yl]-N-[−(1-methyl-1H-pyrazol-5-yl)ethyl]-6,7-dihydro-1′H-spiro[pyrazolo[5,1-c][1,4]oxazine-4,3′-pyrrolidine]-1′-carboxamide (diastereomer 1)
(3′R)-2-[6-amino-5-(trifluoromethyl)pyridin-3-yl]-N-[1-(1-methyl-1H-pyrazol-5-yl)ethyl]-6,7-dihydro-1′H-spiro[pyrazolo[5,1-c][1,4]oxazine-4,3′-pyrrolidine]-1′-carboxamide (diastereomer 2)
(3′R)-2-[6-amino-5-(trifluoromethyl)pyridin-3-yl]-N-[1-(1,3-dimethyl-1H-pyrazol-4-yl)ethyl]-6,7-dihydro-1′H-spiro[pyrazolo[5,1-c][1,4]oxazine-4,3′-pyrrolidine]-1′-carboxamide (diastereomer 1 and diastereomer 2)
(3′R)-2-[6-amino-5-(trifluoromethyl)pyridin-3-yl]-N-[1-(1,3-dimethyl-1H-pyrazol-4-yl)ethyl]-6,7-dihydro-1′H-spiro[pyrazolo[5,1-c][1,4]oxazine-4,3′-pyrrolidine]-1′-carboxamide (diastereomer 1)
(3′R)-2-[6-amino-5-(trifluoromethyl)pyridin-3-yl]-N-[1-(1,3-dimethyl-1H-pyrazol-4-yl)ethyl]-6,7-dihydro-1′H-spiro[pyrazolo[5,1-c][1,4]oxazine-4,3′-pyrrolidine]-1′-carboxamide (diastereomer 2)
(3R)-2′-{6-amino-5-[(1R)-1-(3-cyanophenyl)ethoxy]pyridin-3-yl}-N-ethyl-5′,6′-dihydrospiro[pyrrolidine-3,4′-pyrrolo[1,2-b]pyrazole]-1-carboxamide
(3R)-2′-{6-amino-5-[(1R)-1-(4-cyanophenyl)ethoxy]pyridin-3-yl}-N-ethyl-5′,6′-dihydrospiro[pyrrolidine-3,4′-pyrrolo[1,2-b]pyrazole]-1-carboxamide
(3R)-2′-{6-amino-5-[(1R)-1-(3,5-difluoropyridin-4-yl)ethoxy]pyridin-3-yl}-N-ethyl-5′,6′-dihydrospiro[pyrrolidine-3,4′-pyrrolo[1,2-b]pyrazole]-1-carboxamide
(3R)-2′-[6-amino-5-(1-phenylpropoxy)pyridin-3-yl]-N-ethyl-5′,6′-dihydro-1H-spiro[pyrrolidine-3,4′-pyrrolo[1,2-b]pyrazole]-1-carboxamide (diastereomer 1 and diastereomer 2)
(3R)-2′-[6-amino-5-(1-phenylpropoxy)pyridin-3-yl]-N-ethyl-5′,6′-dihydro-1H-spiro[pyrrolidine-3,4′-pyrrolo[1,2-b]pyrazole]-1-carboxamide (diastereomer 1)
(3R)-2′-[6-amino-5-(1-phenylpropoxy)pyridin-3-yl]-N-ethyl-5′,6′-dihydro-1H-spiro[pyrrolidine-3,4′-pyrrolo[1,2-b]pyrazole]-1-carboxamide (diastereomer 2)
(3R)-2′-{6-amino-5-[(1S)-1-(3-cyanophenyl)ethoxy]pyridin-3-yl}-N-ethyl-5′,6′-dihydrospiro[pyrrolidine-3,4′-pyrrolo[1,2-b]pyrazole]-1-carboxamide
(3R)-2′-{6-amino-5-[(1S)-1-(4-cyanophenyl)ethoxy]pyridin-3-yl}-N-ethyl-5′,6′-dihydrospiro[pyrrolidine-3,4′-pyrrolo[1,2-b]pyrazole]-1-carboxamide
(3R)-2′-{6-amino-5-[(1S)-1-(3,5-difluoropyridin-4-yl)ethoxy]pyridin-3-yl}-N-ethyl-5′,6′-dihydrospiro[pyrrolidine-3,4′-pyrrolo[1,2-b]pyrazole]-1-carboxamide
(3′R)-2-[6-amino-5-(trifluoromethyl)pyridin-3-yl]-N-[1-(1,3,4-trimethyl-1H-pyrazol-5-yl)ethyl]-6,7-dihydro-1′H-spiro[pyrazolo[5,1-c][1,4]oxazine-4,3′-pyrrolidine]-1′-carboxamide (diastereomer 1 and diastereomer 2)
(3′R)-2-[6-amino-5-(trifluoromethyl)pyridin-3-yl]-N-[1-(1,3,4-trimethyl-1H-pyrazol-5-yl)ethyl]-6,7-dihydro-1′H-spiro[pyrazolo[5,1-c][1,4]oxazine-4,3′-pyrrolidine]-1′-carboxamide (diastereomer 1)
(3′R)-2-[6-amino-5-(trifluoromethyl)pyridin-3-yl]-N-[1-(1,3,4-trimethyl-1H-pyrazol-5-yl)ethyl]-6,7-dihydro-1′H-spiro[pyrazolo[5,1-c][1,4]oxazine-4,3′-pyrrolidine]-1′-carboxamide (diastereomer 2)
(3′R)-2-[6-amino-5-(trifluoromethyl)pyridin-3-yl]-N-[1-(1,5-dimethyl-1H-pyrazol-4-yl)ethyl]-6,7-dihydro-1′H-spiro[pyrazolo[5,1-c][1,4]oxazine-4,3′-pyrrolidine]-1′-carboxamide (diastereomer 1 and diastereomer 2)
(3′R)-2-[6-amino-5-(trifluoromethyl)pyridin-3-yl]-N-[1-(1-methyl-1H-pyrazol-4-yl)ethyl]-6,7-dihydro-1′H-spiro[pyrazolo[5,1-c][1,4]oxazine-4,3′-pyrrolidine]-1′-carboxamide (diastereomer 1 and diastereomer 2)
(3R)-2′-{6-amino-5-[(1R)-1-(3-fluoropyridin-2-yl)ethoxy]pyridin-3-yl}-N-(propan-2-yl)-5′,6′-dihydrospiro[pyrrolidine-3,4′-pyrrolo[1,2-b]pyrazole]-1-carboxamide
(3R)-2′-{6-amino-5-[(1R)-1-(3-fluoropyridin-2-yl)ethoxy]pyridin-3-yl}-N-cyclobutyl-5′,6′-dihydrospiro[pyrrolidine-3,4′-pyrrolo[1,2-b]pyrazole]-1-carboxamide
(3R)-2′-{6-amino-5-[(1R)-1-(3,5-difluoropyridin-4-yl)ethoxy]pyridin-3-yl}-N-(propan-2-yl)-5′,6′-dihydrospiro[pyrrolidine-3,4′-pyrrolo[1,2-b]pyrazole]-1-carboxamide
(3R)-2′-{6-amino-5-[(1R)-1-(3,5-difluoropyridin-4-yl)ethoxy]pyridin-3-yl}-N-cyclobutyl-5′,6′-dihydrospiro[pyrrolidine-3,4′-pyrrolo[1,2-b]pyrazole]-1-carboxamide
(3R)-2′-{6-amino-5-[(1R)-1-phenylethoxy]pyridin-3-yl}-N-ethyl-5′,6′-dihydrospiro[pyrrolidine-3,4′-pyrrolo[1,2-b]pyrazole]-1-carboxamide
(3R)-2′-{6-amino-5-[1-(1-methyl-1H-pyrazol-5-yl)ethoxy]pyridin-3-yl}-N-isopropyl-5′,6′-dihydro-1H-spiro[pyrrolidine-3,4′-pyrrolo[1,2-b]pyrazole]-1-carboxamide (diastereomer 1)
(3R)-2′-{6-amino-5-[1-(1-methyl-1H-pyrazol-5-yl)ethoxy]pyridin-3-yl}-N-isopropyl-5′,6′-dihydro-1H-spiro[pyrrolidine-3,4′-pyrrolo[1,2-b]pyrazole]-1-carboxamide (diastereomer 2)
(3R)-2′-{6-amino-5-[1-(1-methyl-1H-pyrazol-5-yl)ethoxy]pyridin-3-yl}-N-cyclobutyl-5′,6′-dihydro-1H-spiro[pyrrolidine-3,4′-pyrrolo[1,2-b]pyrazole]-1-carboxamide (diastereomer 1)
(3R)-2′-{6-amino-5-[1-(1-methyl-1H-pyrazol-5-yl)ethoxy]pyridin-3-yl}-N-cyclobutyl-5′,6′-dihydro-1H-spiro[pyrrolidine-3,4′-pyrrolo[1,2-b]pyrazole]-1-carboxamide (diastereomer 2)
(3R)-2′-{6-amino-5-[(1R)-1-(2-fluorophenyl)ethoxy]pyridin-3-yl}-N-(propan-2-yl)-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1-methyl-1H-pyrazol-5-yl)ethyl]-5′,6′-dihydro-1H-spiro[azetidine-3,4′-pyrrolo[1,2-b]pyrazole]-1-carboxamide (enantiomer 1 and enantiomer 2)
2′-[6-amino-5-(trifluoromethyl)pyridin-3-yl]-N-[(1-methyl-1H-pyrazol-5-yl)ethyl]-5′,6′-dihydro-1H-spiro[azetidine-3,4′-pyrrolo[1,2-b]pyrazole]-1-carboxamide (enantiomer 1)
2′-[6-amino-5-(trifluoromethyl)pyridin-3-yl]-N-[(1-methyl-1H-pyrazol-5-yl)ethyl]-5′,6′-dihydro-1H-spiro[azetidine-3,4′-pyrrolo[1,2-b]pyrazole]-1-carboxamide (enantiomer 2)
2′-[6-amino-5-(trifluoromethyl)pyridin-3-yl]-N-[1-(1,3-dimethyl-1H-pyrazol-4-yl)ethyl]-5′,6′-dihydro-1H-spiro[azetidine-3,4′-pyrrolo[1,2-b]pyrazole]-1-carboxamide (enantiomer 1 and enantiomer 2)
2′-[6-amino-5-(trifluoromethyl)pyridin-3-yl]-N-[1-(1,3-dimethyl-1H-pyrazol-4-yl)ethyl]-5′,6′-dihydro-1H-spiro[azetidine-3,4′-pyrrolo[1,2-b]pyrazole]-1-carboxamide (enantiomer 1)
2′-[6-amino-5-(trifluoromethyl)pyridin-3-yl]-N-[1-(1,3-dimethyl-1H-pyrazol-4-yl)ethyl]-5′,6′-dihydro-1H-spiro[azetidine-3,4′-pyrrolo[1,2-b]pyrazole]-1-carboxamide (enantiomer 2)
(3S)-2′-[6-amino-5-(trifluoromethyl) pyridin-3-yl]-N-[1-(1-methyl-1H-pyrazol-5-yl)ethyl]-5′,6′-dihydro-1H-spiro[pyrrolidine-3,4′-pyrrolo[1,2-b]pyrazole]-1-carboxamide (diastereomer 1 and diastereomer
2)
(3S)-2′-[6-amino-5-(trifluoromethyl) pyridin-3-yl]-N-[1-(1,3-dimethyl-1H-pyrazol-4-yl)ethyl]-5′,6′-dihydro-1H-spiro[pyrrolidine-3,4′-pyrrolo[1,2-b]pyrazole]-1-carboxamide (diastereomer 1 and diastereomer 2)
(3R)-2′-{6-amino-5-[(1R)-1-(2,6-difluorophenyl)ethoxy]pyridin-3-yl}-N-(propan-2-yl)-5′,6′-dihydrospiro[pyrrolidine-3,4′-pyrrolo[1,2-b]pyrazole]-1-carboxamide
(3R)-2′-(6-amino-5-{methyl[(1R)-1-phenylethyl]amino}pyridin-3-yl)-N-ethyl-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[1-(1,3,5-trimethyl-1H-pyrazol-4-yl)ethyl]-5′,6′-dihydro-1H-spiro[azetidine-3,4′-pyrrolo[1,2-b]pyrazole]-1-carboxamide (diastereomer 1 and diastereomer 2)
2′-[6-amino-5-(trifluoromethyl)pyridin-3-yl]-N-[1-(1,3,5-trimethyl-1H-pyrazol-4-yl)ethyl]-5′,6′-dihydro-1H-spiro[azetidine-3,4′-pyrrolo[1,2-b]pyrazole]-1-carboxamide (diastereomer 1)
2′-[6-amino-5-(trifluoromethyl)pyridin-3-yl]-N-[1-(1,3,5-trimethyl-1H-pyrazol-4-yl)ethyl]-5′,6′-dihydro-1H-spiro[azetidine-3,4′-pyrrolo[1,2-b]pyrazole]-1-carboxamide (diastereomer 2)
(3R)-2′-{6-amino-5-[(1R)-1-(4-methylpyridin-2-yl)ethoxy]pyridin-3-yl}-N-(propan-2-yl)-5′,6′-dihydrospiro[pyrrolidine-3,4′-pyrrolo[1,2-b]pyrazole]-1-carboxamide
(3R)-2′-{6-amino-5-[(1R)-1-(4-methylpyridin-2-yl)ethoxy]pyridin-3-yl}-N-cyclobutyl-5′,6′-dihydrospiro[pyrrolidine-3,4′-pyrrolo[1,2-b]pyrazole]-1-carboxamide
(3R)-2′-{6-amino-5-[(1R)-1-(6-methylpyridin-2-yl)ethoxy]pyridin-3-yl}-N-(propan-2-yl)-5′,6′-dihydrospiro[pyrrolidine-3,4′-pyrrolo[1,2-b]pyrazole]-1-carboxamide
(3R)-2′-{6-amino-5-[(1R)-1-(6-methylpyridin-2-yl)ethoxy]pyridin-3-yl}-N-cyclobutyl-5′,6′-dihydrospiro[pyrrolidine-3,4′-pyrrolo[1,2-b]pyrazole]-1-carboxamide
(3S)-2′-[6-amino-5-(trifluoromethyl)pyridin-3-yl]-N-[2-(3-fluoropyridin-2-yl)propan-2-yl]-5′,6′-dihydrospiro[pyrrolidine-3,4′-pyrrolo[1,2-b]pyrazole]-1-carboxamide
(3S)-2′-[6-amino-5-(trifluoromethyl) pyridin-3-yl]-N-[1-(1,3,5-trimethyl-1H-pyrazol-4-yl)ethyl]-5′,6′-dihydro-1H-spiro[pyrrolidine-3,4′-pyrrolo[1,2-b]pyrazole]-1-carboxamide (diastereomer 1 and diastereomer 2)
(3R)-2′-[6-amino-5-(trifluoromethyl)pyridin-3-yl]-N-[1-(1,3,5-trimethyl-1H-pyrazol-4-yl)ethyl]-5′,6′-dihydro-1H-spiro[pyrrolidine-3,4′-pyrrolo[1,2-b]pyrazole]-1-carboxamide (diastereomer 1 and diastereomer 2)
(3R)-2′-[6-amino-5-(trifluoromethyl)pyridin-3-yl]-N-[1-(1,3,5-trimethyl-1H-pyrazol-4-yl)ethyl]-5′,6′-dihydro-1H-spiro[pyrrolidine-3,4′-pyrrolo[1,2-b]pyrazole]-1-carboxamide (diastereomer 1)
(3R)-2′-[6-amino-5-(trifluoromethyl)pyridin-3-yl]-N-[1-(1,3,5-trimethyl-1H-pyrazol-4-yl)ethyl]-5′,6′-dihydro-1H-spiro[pyrrolidine-3,4′-pyrrolo[1,2-b]pyrazole]-1-carboxamide (diastereomer 2)
(2′-{6-amino-5-[(1R)-1-phenylethoxy]pyridin-3-yl}-5′,6′-dihydrospiro[azetidine-3,4′-pyrrolo[1,2-b]pyrazol]-1-yl)(cyclopropyl)methanone
2′-[6-amino-5-(trifluoromethyl)pyridin-3-yl]-N-[1-(1-ethyl-1H-pyrazol-3-yl)ethyl]-5′,6′-dihydro-1H-spiro[azetidine-3,4′-pyrrolo[1,2-b]pyrazole]-1-carboxamide (enantiomer 1)
2′-[6-amino-5-(trifluoromethyl)pyridin-3-yl]-N-[1-(1-ethyl-1H-pyrazol-3-yl)ethyl]-5′,6′-dihydro-1H-spiro[azetidine-3,4′-pyrrolo[1,2-b]pyrazole]-1-carboxamide (enantiomer 2)
(3S)-2′-[6-amino-5-(trifluoromethyl)pyridin-3-yl]-N-[(1-ethyl-1H-pyrazol-3-yl)ethyl]-5′,6′-dihydro-1H-spiro[pyrrolidine-3,4′-pyrrolo[1,2-b]pyrazole]-1-carboxamide (diastereomer 1)
(3S)-2′-[6-amino-5-(trifluoromethyl)pyridin-3-yl]-N-[1-(1-ethyl-1H-pyrazol-3-yl)ethyl]-5′,6′-dihydro-1H-spiro[pyrrolidine-3,4′-pyrrolo[1,2-b]pyrazole]-1-carboxamide (diastereomer 2)
5-{(3′R)-1′-[cyclopropyl(1H-1,2,4-triazol-5-yl)methyl]-6,7-dihydrospiro[pyrazolo[5,1-c][1,4]oxazine-4,3′-pyrrolidin]-2-yl}-3-(trifluoromethyl)pyridin-2-amine (diastereomer 1 and diastereomer 2)
5-{(3′R)-1′-[cyclopropyl(1H-1,2,4-triazol-5-yl)methyl]-6,7-dihydrospiro[pyrazolo[5,1-c][1,4]oxazine-4,3′-pyrrolidin]-2-yl}-3-(trifluoromethyl)pyridin-2-amine (diastereomer 1)
5-{(3′R)-1′-[cyclopropyl(1H-1,2,4-triazol-5-yl)methyl]-6,7-dihydrospiro[pyrazolo[5,1-c][1,4]oxazine-4,3′-pyrrolidin]-2-yl}-3-(trifluoromethyl)pyridin-2-amine (diastereomer 2)
5-{(3R)-1-[1-(1H-1,2,4-triazol-5-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[1-(1H-1,2,4-triazol-5-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[1-(1H-1,2,4-triazol-5-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3R)-1-[cyclopropyl(1H-1,2,4-triazol-5-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3′R)-1′-[1-(1H-1,2,4-triazol-5-yl)propyl]-6,7-dihydrospiro[pyrazolo[5,1-c][1,4]oxazine-4,3′-pyrrolidin]-2-yl}-3-(trifluoromethyl)pyridin-2-amine (diastereomer 1 and diastereomer 2)
5-{(3′R)-1′-[1-(1H-1,2,4-triazol-5-yl)propyl]-6,7-dihydrospiro[pyrazolo[5,1-c][1,4]oxazine-4,3′-pyrrolidin]-2-yl}-3-(trifluoromethyl)pyridin-2-amine (diastereomer 1)
5-{(3′R)-1′-[1-(1H-1,2,4-Triazol-5-yl)propyl]-6,7-dihydrospiro[pyrazolo[5,1-c][1,4]oxazine-4,3′-pyrrolidin]-2-yl}-3-(trifluoromethyl)pyridin-2-amine (diastereomer 2)
5-{(3′R)-1′-[2,2-difluoro-1-(1H-imidazol-2-yl)ethyl]-6,7-dihydrospiro[pyrazolo[5,1-c][1,4]oxazine-4,3′-pyrrolidin]-2-yl}-3-(trifluoromethyl)pyridin-2-amine (diastereomer 2)
5-{(3′R)-1′-[2,2-difluoro-1-(1H-imidazol-2-yl)ethyl]-6,7-dihydrospiro[pyrazolo[5,1-c][1,4]oxazine-4,3′-pyrrolidin]-2-yl}-3-(trifluoromethyl)pyridin-2-amine (diastereomer 1)
5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}-3-(difluoromethyl)pyridin-2-amine (diastereomer 1 and diastereomer 2)
5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}-3-(difluoromethyl)pyridin-2-amine (diastereomer 1)
5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}-3-(difluoromethyl)pyridin-2-amine (diastereomer 2)
2-amino-5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}nicotinonitrile (diastereomer 1 and diastereomer 2)
2-amino-5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}nicotinonitrile (diastereomer 1)
2-amino-5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}nicotinonitrile (diastereomer 2)
5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}-3-(difluoromethoxy)pyridin-2-amine (diastereomer 1 and diastereomer 2)
5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}-3-(difluoromethoxy)pyridin-2-amine (diastereomer 1)
5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}-3-(difluoromethoxy)pyridin-2-amine (diastereomer 2)
5-{1-[1-(1H-imidazol-2-yl)propyl]-5′,6′-dihydrospiro[azetidine-3,4′-pyrrolo[1,2-b]pyrazol]-2′-yl}-3-(trifluoromethyl)pyridin-2-amine (enantiomer 1 and enantiomer 2)
5-{1-[1-(1H-imidazol-2-yl)propyl]-5′,6′-dihydrospiro[azetidine-3,4′-pyrrolo[1,2-b]pyrazol]-2′-yl}-3-(trifluoromethyl)pyridin-2-amine (enantiomer 1)
5-{1-[1-(1H-imidazol-2-yl)propyl]-5′,6′-dihydrospiro[azetidine-3,4′-pyrrolo[1,2-b]pyrazol]-2′-yl}-3-(trifluoromethyl)pyridin-2-amine (enantiomer 2)
5-{(3′R)-1′-[cyclobutyl(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine (diastereomer 1 and diastereomer 2)
5-{(3′R)-1′-[cyclobutyl(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine (diastereomer 1)
5-{(3′R)-1′-[cyclobutyl(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine (diastereomer 2)
5-{(3′R)-1′-[1-(1H-imidazol-2-yl)-2-methylpropyl]-6,7-dihydrospiro[pyrazolo[5,1-c][1,4]oxazine-4,3′-pyrrolidin]-2-yl}-3-(trifluoromethyl)pyridin-2-amine (diastereomer 1 and diastereomer 2)
5-{(3′R)-1′-[1-(1H-imidazol-2-yl)-2-methylpropyl]-6,7-dihydrospiro[pyrazolo[5,1-c][1,4]oxazine-4,3′-pyrrolidin]-2-yl}-3-(trifluoromethyl)pyridin-2-amine (diastereomer 1)
5-{(3′R)-1′-[1-(1H-imidazol-2-yl)-2-methylpropyl]-6,7-dihydrospiro[pyrazolo[5,1-c][1,4]oxazine-4,3′-pyrrolidin]-2-yl}-3-(trifluoromethyl)pyridin-2-amine (diastereomer 2)
5-{(3R)-1-[cyclobutyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[cyclobutyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[cyclobutyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3R)-1-[1-(1H-imidazol-2-yl)-2,2-dimethyl propyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{1-[cyclopropyl(1H-imidazol-2-yl)methyl]-5′,6′-dihydrospiro[azetidine-3,4′-pyrrolo[1,2-b]pyrazol]-2′-yl}-3-(trifluoromethyl)pyridin-2-amine (enantiomer 1 and enantiomer 2)
5-{1-[cyclopropyl(1H-imidazol-2-yl)methyl]-5′,6′-dihydrospiro[azetidine-3,4′-pyrrolo[1,2-b]pyrazol]-2′-yl}-3-(trifluoromethyl)pyridin-2-amine (enantiomer 1)
5-{1-[cyclopropyl(1H-imidazol-2-yl)methyl]-5′,6′-dihydrospiro[azetidine-3,4′-pyrrolo[1,2-b]pyrazol]-2′-yl}-3-(trifluoromethyl)pyridin-2-amine (enantiomer 2)
5-{(3R)-1-[2,2-difluoro-1-(1H-imidazol-2-yl)ethyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[2,2-difluoro-1-(1H-imidazol-2-yl)ethyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[2,2-difluoro-1-(1H-imidazol-2-yl)ethyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3S)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3S)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3S)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine (diastereomer 1 and diastereomer 2)
5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine (diastereomer 1)
5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine (diastereomer 2)
2-amino-5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}nicotinonitrile (diastereomer 1 and diastereomer 2)
2-amino-5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}nicotinonitrile (diastereomer 1)
2-amino-5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}nicotinonitrile (diastereomer 2)
5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(difluoromethoxy)pyridin-2-amine (diastereomer 1 and diastereomer 2)
5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(difluoromethoxy)pyridin-2-amine (diastereomer 1)
5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(difluoromethoxy)pyridin-2-amine (diastereomer 2)
2-amino-5-{(3R)-1-[1-(1H-imidazol-2-yl)propyl]-5′,6′-dihydrospiro[pyrrolidine-3,4′-pyrrolo[1,2-b]pyrazol]-2′-yl}nicotinonitrile (diastereomer 1 and diastereomer 2)
2-amino-5-{(3R)-1-[1-(1H-imidazol-2-yl)propyl]-5′,6′-dihydrospiro[pyrrolidine-3,4′-pyrrolo[1,2-b]pyrazol]-2′-yl}nicotinonitrile (diastereomer 1)
2-amino-5-{(3R)-1-[1-(1H-imidazol-2-yl)propyl]-5′,6′-dihydrospiro[pyrrolidine-3,4′-pyrrolo[1,2-b]pyrazol]-2′-yl}nicotinonitrile (diastereomer 2)
5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethoxy)pyridin-2-amine (diastereomer 1 and diastereomer 2)
5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethoxy)pyridin-2-amine (diastereomer 1)
5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethoxy)pyridin-2-amine (diastereomer 2)
3-(difluoromethoxy)-5-{(3R)-1-[1-(1H-imidazol-2-yl)propyl]-5′,6′-dihydrospiro[pyrrolidine-3,4′-pyrrolo[1,2-b]pyrazol]-2′-yl}pyridin-2-amine (diastereomer 1 and diastereomer 2)
3-(difluoromethoxy)-5-{(3R)-1-[1-(1H-imidazol-2-yl)propyl]-5′,6′-dihydrospiro[pyrrolidine-3,4′-pyrrolo[1,2-b]pyrazol]-2′-yl}pyridin-2-amine (diastereomer 1)
3-(difluoromethoxy)-5-{(3R)-1-[1-(1H-imidazol-2-yl)propyl]-5′,6′-dihydrospiro[pyrrolidine-3,4′-pyrrolo[1,2-b]pyrazol]-2′-yl}pyridin-2-amine (diastereomer 2)
5-{(3R)-1-[1-(1H-imidazol-2-yl)propyl]-5′,6′-dihydrospiro[pyrrolidine-3,4′-pyrrolo[1,2-b]pyrazol]-2′-yl}-3-(trifluoromethoxy)pyridin-2-amine (diastereomer 1 and diastereomer 2)
5-{(3R)-1-[1-(1H-imidazol-2-yl)propyl]-5′,6′-dihydrospiro[pyrrolidine-3,4′-pyrrolo[1,2-b]pyrazol]-2′-yl}-3-(trifluoromethoxy)pyridin-2-amine (diastereomer 1)
5-{(3R)-1-[1-(1H-imidazol-2-yl)propyl]-5′,6′-dihydrospiro[pyrrolidine-3,4′-pyrrolo[1,2-b]pyrazol]-2′-yl}-3-(trifluoromethoxy)pyridin-2-amine (diastereomer 2)
5-{(3R)-1-[1-(1H-imidazol-2-yl)-2-methylpropyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[1-(1H-imidazol-2-yl)-2-methylpropyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[1-(1H-imidazol-2-yl)-2-methyl propyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3S)-1-[1-(1H-imidazol-2-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3S)-1-[1-(1H-imidazol-2-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3S)-1-[1-(1H-imidazol-2-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3′R)-1′-[1-(1H-imidazol-2-yl)propyl]-6,7-dihydrospiro[pyrazolo[5,1-c][1,4]oxazine-4,3′-pyrrolidin]-2-yl}-3-(trifluoromethyl)pyridin-2-amine (diastereomer 1 and diastereomer 2)
5-{(3′R)-1′-[1-(1H-imidazol-2-yl)propyl]-6,7-dihydrospiro[pyrazolo[5,1-c][1,4]oxazine-4,3′-pyrrolidin]-2-yl}-3-(trifluoromethyl)pyridin-2-amine (diastereomer 1)
5-{(3′R)-1′-[1-(1H-imidazol-2-yl)propyl]-6,7-dihydrospiro[pyrazolo[5,1-c][1,4]oxazine-4,3′-pyrrolidin]-2-yl}-3-(trifluoromethyl)pyridin-2-amine (diastereomer 2)
{(3R)-2′-[6-amino-5-(trifluoromethyl)pyridin-3-yl]-5′,6′-dihydrospiro[pyrrolidine-3,4′-pyrrolo[1,2-b]pyrazol]-1-yl}(1H-imidazol-2-yl)methanone
{(3R)-2′-[6-amino-5-(trifluoromethyl)pyridin-3-yl]-5′,6′-dihydrospiro[pyrrolidine-3,4′-pyrrolo[1,2-b]pyrazol]-1-yl}(1-methyl-1H-imidazol-2-yl)methanone
3-[(1R)-1-(2,6-dichloro-3-fluorophenyl)ethoxy]-5-{(3R)-1-[(1H-imidazol-2-yl)methyl]-5′,6′-dihydrospiro[pyrrolidine-3,4′-pyrrolo[1,2-b]pyrazol]-2′-yl}pyridin-2-amine
5-{(3R)-1-[1-(1H-imidazol-2-yl)ethyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[1-(1H-imidazol-2-yl)ethyl]-5′,6′-dihydrospiro[pyrrolidine-3,4′-pyrrolo[1,2-b]pyrazol]-2′-yl}-3-(trifluoromethyl)pyridin-2-amine (diastereomer 2)
(3R)-2′-[6-amino-5-(trifluoromethyl)pyridin-3-yl]-N-[1-(3-chloropyridin-4-yl)cyclobutyl]-5′,6′-dihydrospiro[pyrrolidine-3,4′-pyrrolo[1,2-b]pyrazole]-1-carboxamide
(3R)-2′-[6-amino-5-(trifluoromethyl)pyridin-3-yl]-N-[2-(3-chloropyridin-4-yl)propan-2-yl]-5′,6′-dihydrospiro[pyrrolidine-3,4′-pyrrolo[1,2-b]pyrazole]-1-carboxamide
(3R)-2′-[6-amino-5-(trifluoromethyl)pyridin-3-yl]-N-[1-(pyridin-4-yl)cyclobutyl]-5′,6′-dihydrospiro[pyrrolidine-3,4′-pyrrolo[1,2-b]pyrazole]-1-carboxamide
(3R)-2′-[6-amino-5-(trifluoromethyl)pyridin-3-yl]-N-[1-(pyridin-3-yl)cyclobutyl]-5′,6′-dihydrospiro[pyrrolidine-3,4′-pyrrolo[1,2-b]pyrazole]-1-carboxamide
(3R)-2′-[6-amino-5-(trifluoromethyl)pyridin-3-yl]-N-[(1S)-1-(3-chloropyridin-4-yl)ethyl]-5′,6′-dihydrospiro[pyrrolidine-3,4′-pyrrolo[1,2-b]pyrazole]-1-carboxamide
2′-(6-amino-5-cyanopyridin-3-yl)-N-[1-(pyridin-4-yl)cyclobutyl]-5′,6′-dihydrospiro[pyrrolidine-3,4′-pyrrolo[1,2-b]pyrazole]-1-carboxamide (enantiomer 2)
(3R)-2′-[6-amino-5-(trifluoromethyl)pyridin-3-yl]-N-[1-(3-methylpyridin-4-yl)ethyl]-5′,6′-dihydro-1H-spiro[pyrrolidine-3,4′-pyrrolo[1,2-b]pyrazole]-1-carboxamide (diastereomer 1 and diastereomer 2)
(3R)-2′-[6-amino-5-(trifluoromethyl)pyridin-3-yl]-N-[cyclopropyl(phenyl)methyl]-5′,6′-dihydrospiro[pyrrolidine-3,4′-pyrrolo[1,2-b]pyrazole]-1-carboxamide (diastereomer 1)
(3R)-2′-[6-amino-5-(trifluoromethyl)pyridin-3-yl]-N-[cyclopropyl(phenyl)methyl]-5′,6′-dihydrospiro[pyrrolidine-3,4′-pyrrolo[1,2-b]pyrazole]-1-carboxamide (diastereomer 2)
(3R)-2′-[6-amino-5-(trifluoromethyl)pyridin-3-yl]-N-[1-(pyridin-2-yl)cyclobutyl]-5′,6′-dihydrospiro[pyrrolidine-3,4′-pyrrolo[1,2-b]pyrazole]-1-carboxamide
(3R)-2′-[6-amino-5-(trifluoromethyl)pyridin-3-yl]-N-[2-methyl-1-(pyridin-4-yl)propyl]-5′,6′-dihydro-1H-spiro[pyrrolidine-3,4′-pyrrolo[1,2-b]pyrazole]-1-carboxamide (diastereomer 1 and diastereomer
2)
(3R)-2′-[6-amino-5-(trifluoromethyl)pyridin-3-yl]-N-[2-methyl-1-(pyridin-4-yl)propyl]-5′,6′-dihydro-1H-spiro[pyrrolidine-3,4′-pyrrolo[1,2-b]pyrazole]-1-carboxamide (diastereomer 1)
(3R)-2′-[6-amino-5-(trifluoromethyl)pyridin-3-yl]-N-[2-methyl-1-(pyridin-4-yl)propyl]-5′,6′-dihydro-1H-spiro[pyrrolidine-3,4′-pyrrolo[1,2-b]pyrazole]-1-carboxamide (diastereomer 2)
(3R)-2′-[6-amino-5-(trifluoromethyl)pyridin-3-yl]-N-[1-(3-fluoropyridin-4-yl)ethyl]-5′,6′-dihydro-1H-spiro[pyrrolidine-3,4′-pyrrolo[1,2-b]pyrazole]-1-carboxamide (diastereomer 1 and diastereomer 2)
(3R)-2′-[6-amino-5-(trifluoromethyl)pyridin-3-yl]-N-[1-(3-fluoropyridin-4-yl)ethyl]-5′,6′-dihydro-1H-spiro[pyrrolidine-3,4′-pyrrolo[1,2-b]pyrazole]-1-carboxamide (diastereomer 1)
(3R)-2′-[6-amino-5-(trifluoromethyl)pyridin-3-yl]-N-[1-(3-fluoropyridin-4-yl)ethyl]-5′,6′-dihydro-1H-spiro[pyrrolidine-3,4′-pyrrolo[1,2-b]pyrazole]-1-carboxamide (diastereomer 2)
(3R)-2′-[6-amino-5-(trifluoromethyl)pyridin-3-yl]-N-[(1R)-1-(3-chloropyridin-4-yl)ethyl]-5′,6′-dihydrospiro[pyrrolidine-3,4′-pyrrolo[1,2-b]pyrazole]-1-carboxamide
(3R)-2′-[6-amino-5-(trifluoromethyl)pyridin-3-yl]-N-[1-(2-methylpyridin-4-yl)ethyl]-5′,6′-dihydro-1H-spiro[pyrrolidine-3,4′-pyrrolo[1,2-b]pyrazole]-1-carboxamide (diastereomer 1 and diastereomer 2)
(3R)-2′-[6-amino-5-(trifluoromethyl)pyridin-3-yl]-N-[1-(3-methoxypyridin-4-yl)ethyl]-5′,6′-dihydro-1H-spiro[pyrrolidine-3,4′-pyrrolo[1,2-b]pyrazole]-1-carboxamide (diastereomer 1 and diastereomer
2)
(3R)-2′-[6-amino-5-(trifluoromethyl)pyridin-3-yl]-N-[1-(pyridin-4-yl)cyclopropyl]-5′,6′-dihydrospiro[pyrrolidine-3,4′-pyrrolo[1,2-b]pyrazole]-1-carboxamide
(3R)-2′-{6-amino-5-[(1R)-1-(2-methoxyphenyl)ethoxy]pyridin-3-yl}-N-ethyl-5′,6′-dihydrospiro[pyrrolidine-3,4′-pyrrolo[1,2-b]pyrazole]-1-carboxamide
3-(1-{(3R)-2′-[6-amino-5-(trifluoromethyl)pyridin-3-yl]-5′,6′-dihydro-1H-spiro[pyrrolidine-3,4′-pyrrolo[1,2-b]pyrazol]-1-yl}propyl)benzonitrile (diastereomer 1 and diastereomer 2)
3-(1-{(3R)-2′-[6-amino-5-(trifluoromethyl)pyridin-3-yl]-5′,6′-dihydro-1H-spiro[pyrrolidine-3,4′-pyrrolo[1,2-b]pyrazol]-1-yl}propyl)benzonitrile (diastereomer 1)
3-(1-{(3R)-2′-[6-amino-5-(trifluoromethyl)pyridin-3-yl]-5′,6′-dihydro-1H-spiro[pyrrolidine-3,4′-pyrrolo[1,2-b]pyrazol]-1-yl}propyl)benzonitrile (diastereomer 2)
(3S)-2′-(6-amino-5-cyanopyridin-3-yl)-N-[2-(3-chloropyridin-4-yl)propan-2-yl]-5′,6′-dihydrospiro[pyrrolidine-3,4′-pyrrolo[1,2-b]pyrazole]-1-carboxamide (enantiomer 1)
(3R)-2′-(6-amino-5-cyanopyridin-3-yl)-N-[2-(3-chloropyridin-4-yl)propan-2-yl]-5′,6′-dihydrospiro[pyrrolidine-3,4′-pyrrolo[1,2-b]pyrazole]-1-carboxamide (enantiomer 2)
(3R)-2′-{6-amino-5-[(1R)-1-(3-chlorophenyl)ethoxy]pyridin-3-yl}-N-(propan-2-yl)-5′,6′-dihydrospiro[pyrrolidine-3,4′-pyrrolo[1,2-b]pyrazole]-1-carboxamide
(3R)-2′-{6-amino-5-[1-(2,6-dichlorophenyl)ethoxy]pyridin-3-yl}-N-ethyl-5′,6′-dihydro-1H-spiro[pyrrolidine-3,4′-pyrrolo[1,2-b]pyrazole]-1-carboxamide (diastereomer 1 and diastereomer 2)
(3R)-2′-{6-amino-5-[1-(2,6-dichlorophenyl)ethoxy]pyridin-3-yl}-N-ethyl-5′,6′-dihydro-1H-spiro[pyrrolidine-3,4′-pyrrolo[1,2-b]pyrazole]-1-carboxamide (diastereomer 1)
(3R)-2′-{6-amino-5-[1-(2,6-dichlorophenyl)ethoxy]pyridin-3-yl}-N-ethyl-5′,6′-dihydro-1H-spiro[pyrrolidine-3,4′-pyrrolo[1,2-b]pyrazole]-1-carboxamide (diastereomer 2)
5-{(3R)-1-[1-(1H-pyrrol-2-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
(3R)-2′-[6-amino-5-(trifluoromethyl)pyridin-3-yl]-N-[1-(2-methoxypyridin-4-yl)ethyl]-5′,6′-dihydro-1H-spiro[pyrrolidine-3,4′-pyrrolo[1,2-b]pyrazole]-1-carboxamide (diastereomer 1 and diastereomer
2)
(3R)-2′-[6-amino-5-(trifluoromethyl)pyridin-3-yl]-N-[1-(1-methyl-1H-pyrazol-5-yl)ethyl]-5′,6′-dihydrospiro[pyrrolidine-3,4′-pyrrolo[1,2-b]pyrazole]-1-carboxamide (diastereomer 1)
(3R)-2′-[6-amino-5-(trifluoromethyl)pyridin-3-yl]-N-[1-(1-methyl-1H-pyrazol-5-yl)ethyl]-5′,6′-dihydrospiro[pyrrolidine-3,4′-pyrrolo[1,2-b]pyrazole]-1-carboxamide (diastereomer 2)
3-(1-{(3R)-2′-[6-amino-5-(trifluoromethyl)pyridin-3-yl]-5′,6′-dihydro-1H-spiro[pyrrolidine-3,4′-pyrrolo[1,2-b]pyrazol]-1-yl}propyl)benzamide (diastereomer 1 and diastereomer 2)
3-(1-{(3R)-2′-[6-amino-5-(trifluoromethyl)pyridin-3-yl]-5′,6′-dihydrospiro[pyrrolidine-3,4′-pyrrolo[1,
b]pyrazol]-1-yl}propyl)benzamide (diastereomer 1)
3-(1-{(3R)-2′-[6-amino-5-(trifluoromethyl)pyridin-3-yl]-5′,6′-dihydrospiro[pyrrolidine-3,4′-pyrrolo[1,2-b]pyrazol]-1-yl}propyl)benzamide (diastereomer 2)
(3R)-2′-{6-amino-5-[(1R)-1-(1,3-thiazol-2-yl)ethoxy]pyridin-3-yl}-N-cyclobutyl-5′,6′-dihydrospiro[pyrrolidine-3,4′-pyrrolo[1,2-b]pyrazole]-1-carboxamide
(3R)-2′-{6-amino-5-[(1R)-1-(3-chlorophenyl)ethoxy]pyridin-3-yl}-N-ethyl-5′,6′-dihydrospiro[pyrrolidine-3,4′-pyrrolo[1,2-b]pyrazole]-1-carboxamide
(3R)-2′-[6-amino-5-(trifluoromethyl)pyridin-3-yl]-N-[1-(2-chloro-3-fluoropyridin-4-yl)ethyl]-5′,6′-dihydro-1H-spiro[pyrrolidine-3,4′-pyrrolo[1,2-b]pyrazole]-1-carboxamide (diastereomer 1 and diastereomer 2)
(3R)-2′-[6-amino-5-(trifluoromethyl)pyridin-3-yl]-N-{[3-(methylsulfanyl)pyridin-4-yl]methyl}-5′,6′-dihydrospiro[pyrrolidine-3,4′-pyrrolo[1,2-b]pyrazole]-1-carboxamide
(3R)-2′-{6-amino-5-[(1R)-1-(1,3-thiazol-2-yl)ethoxy]pyridin-3-yl}-N-ethyl-5′,6′-dihydrospiro[pyrrolidine-3,4′-pyrrolo[1,2-b]pyrazole]-1-carboxamide
(3R)-2′-[6-amino-5-(trifluoromethyl)pyridin-3-yl]-N-[1-(1,3-dimethyl-1H-pyrazol-4-yl)ethyl]-5′,6′-dihydrospiro[pyrrolidine-3,4′-pyrrolo[1,2-b]pyrazole]-1-carboxamide (diastereomer 1)
(3R)-2′-[6-amino-5-(trifluoromethyl)pyridin-3-yl]-N-[1-(1,3-dimethyl-1H-pyrazol-4-yl)ethyl]-5′,6′-dihydrospiro[pyrrolidine-3,4′-pyrrolo[1,2-b]pyrazole]-1-carboxamide (diastereomer 2)
(3R)-2′-{6-amino-5-[(1R)-1-(3-fluorophenyl)ethoxy]pyridin-3-yl}-N-(propan-2-yl)-5′,6′-dihydrospiro[pyrrolidine-3,4′-pyrrolo[1,2-b]pyrazole]-1-carboxamide
(3R)-2′-[6-amino-5-(trifluoromethyl)pyridin-3-yl]-N-[1-(pyridin-2-yl)cyclopropyl]-5′,6′-dihydrospiro[pyrrolidine-3,4′-pyrrolo[1,2-b]pyrazole]-1-carboxamide
5-{(3R)-1-[1-(pyrimidin-5-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[1-(pyrimidin-5-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[1-(pyrimidin-5-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 2)
(3R)-2′-{6-amino-5-[(1R)-1-(1,3-thiazol-2-yl)ethoxy]pyridin-3-yl}-N-(propan-2-yl)-5′,6′-dihydrospiro[pyrrolidine-3,4′-pyrrolo[1,2-b]pyrazole]-1-carboxamide
(3R)-2′-[6-amino-5-(trifluoromethyl)pyridin-3-yl]-N-[(3-methylpyridin-4-yl)methyl]-5′,6′-dihydrospiro[pyrrolidine-3,4′-pyrrolo[1,2-b]pyrazole]-1-carboxamide
5-{(3R)-1-[1-(6-methoxypyridin-2-yl)ethyl]-5′,6′-dihydrospiro[pyrrolidine-3,4′-pyrrolo[1,2-b]pyrazol]2′-yl}-3-(trifluoromethyl)pyridin-2-amine (diastereomer 1)
5-{(3R)-1-[1-(6-methoxypyridin-2-yl)ethyl]-5′,6′-dihydrospiro[pyrrolidine-3,4′-pyrrolo[1,2-b]pyrazol]-2′-yl}-3-(trifluoromethyl)pyridin-2-amine (diastereomer 2)
(3R)-2′-(6-amino-5-{1-[3-(3-hydroxy-3-methylbut-1-yn-1-yl)phenyl]ethoxy}pyridin-3-yl)-N-ethyl-5′,6′-dihydro-1H-spiro[pyrrolidine-3,4′-pyrrolo[1,2-b]pyrazole]-1-carboxamide (diastereomer 1 and diastereomer 2)
(3R)-2′-{6-amino-5-[(1R)-1-(pyridin-3-yl)ethoxy]pyridin-3-yl}-N-cyclobutyl-5′,6′-dihydrospiro[pyrrolidine-3,4′-pyrrolo[1,2-b]pyrazole]-1-carboxamide
(3R)-2′-{6-amino-5-[1-(1,3-oxazol-2-yl)ethoxy]pyridin-3-yl}-N-ethyl-5′,6′-dihydro-1H-spiro[pyrrolidine-3,4′-pyrrolo[1,2-b]pyrazole]-1-carboxamide (diastereomer 1 and diastereomer 2)
(3R)-2′-{6-amino-5-[(1R)-1-(3-fluorophenyl)ethoxy]pyridin-3-yl}-N-ethyl-5′,6′-dihydrospiro[pyrrolidine-3,4′-pyrrolo[1,2-b]pyrazole]-1-carboxamide
(3R)-2′-[6-amino-5-(6,7-dihydro-5H-cyclopenta[b]pyridin-7-yloxy)pyridin-3-yl]-N-ethyl-5′,6′-dihydro-1H-spiro[pyrrolidine-3,4′-pyrrolo[1,2-b]pyrazole]-1-carboxamide (diastereomer 2)
5-{(3R)-1-[cyclopropyl(5-fluoropyridin-3-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
(3R)-2′-{6-amino-5-[(1R)-1-(pyrimidin-2-yl)ethoxy]pyridin-3-yl}-N-(propan-2-yl)-5′,6′-dihydrospiro[pyrrolidine-3,4′-pyrrolo[1,2-b]pyrazole]-1-carboxamide
(3R)-2′-{6-amino-5-[(1R)-1-(4-fluorophenyl)ethoxy]pyridin-3-yl}-N-ethyl-5′,6′-dihydrospiro[pyrrolidine-3,4′-pyrrolo[1,2-b]pyrazole]-1-carboxamide
(3R)-2′-{6-amino-5-[(1R)-1-(4-fluorophenyl)ethoxy]pyridin-3-yl}-N-(propan-2-yl)-5′,6′-dihydrospiro[pyrrolidine-3,4′-pyrrolo[1,2-b]pyrazole]-1-carboxamide
5-{(3R)-1-[1-(1,3-thiazol-2-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[1-(1,3-thiazol-2-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[1-(1,3-thiazol-2-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 2)
(3R)-2′-{6-amino-5-[(1R)-1-(4-chlorophenyl)ethoxy]pyridin-3-yl}-N-ethyl-5′,6′-dihydrospiro[pyrrolidine-3,4′-pyrrolo[1,2-b]pyrazole]-1-carboxamide
5-{(3R)-1-[1-(dimethylamino)propan-2-yl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
(3R)-2′-{6-amino-5-[(1R)-1-(1,2-thiazol-5-yl)ethoxy]pyridin-3-yl}-N-ethyl-5′,6′-dihydrospiro[pyrrolidine-3,4′-pyrrolo[1,2-b]pyrazole]-1-carboxamide
6-({(3R)-2′-[6-amino-5-(trifluoromethyl) pyridin-3-yl]-5′,6′-dihydrospiro[pyrrolidine-3,4′-pyrrolo[1,2-b]pyrazol]-1-yl}methyl)pyridin-2(1H)-one
(3R)-2′-{6-amino-5-[(1R)-1-(pyridin-3-yl)ethoxy]pyridin-3-yl}-N-(propan-2-yl)-5′,6′-dihydrospiro[pyrrolidine-3,4′-pyrrolo[1,2-b]pyrazole]-1-carboxamide
2′-[6-amino-5-(6,7-dihydro-5H-cyclopenta[b]pyridin-7-yloxy)pyridin-3-yl]-N-ethyl-5′,6′-dihydro-1H-spiro[pyrrolidine-3,4′-pyrrolo[1,2-b]pyrazole]-1-carboxamide (4 isomers)
(3R)-2′-{6-amino-5-[1-(1,3-oxazol-4-yl)ethoxy]pyridin-3-yl}-N-isopropyl-5′,6′-dihydro-1H-spiro[pyrrolidine-3,4′-pyrrolo[1,2-b]pyrazole]-1-carboxamide (diastereomer 1 and diastereomer 2)
5-{(3R)-1-[(1,3-thiazol-2-yl)methyl]-5′,6′-dihydrospiro[pyrrolidine-3,4′-pyrrolo[1,2-b]pyrazol]-2′-yl}-3-(trifluoromethyl)pyridin-2-amine
6-({(3′R)-2-[6-amino-5-(trifluoromethyl)pyridin-3-yl]-6,7-dihydrospiro[pyrazolo[5,1-c][1,4]oxazine-4,3′-pyrrolidin]-1′-yl}methyl)pyridin-2(1H)-one
(3S)-2′-[6-amino-5-(trifluoromethyl)pyridin-3-yl]-N-[cyclopropyl(phenyl)methyl]-5′,6′-dihydrospiro[pyrrolidine-3,4′-pyrrolo[1,2-b]pyrazole]-1-carboxamide (diastereomer 1)
(3S)-2′-[6-amino-5-(trifluoromethyl)pyridin-3-yl]-N-[cyclopropyl(phenyl)methyl]-5′,6′-dihydrospiro[pyrrolidine-3,4′-pyrrolo[1,2-b]pyrazole]-1-carboxamide (diastereomer 2)
(3R)-2′-[6-amino-5-(6,7-dihydro-5H-cyclopenta[b]pyridin-7-yloxy)pyridin-3-yl]-N-ethyl-5′,6′-dihydro-1H-spiro[pyrrolidine-3,4′-pyrrolo[1,2-b]pyrazole]-1-carboxamide (diastereomer 1)
2′-{6-amino-5-[(1R)-1-(pyridin-2-yl)ethoxy]pyridin-3-yl}-N-ethyl-5′,6′-dihydrospiro[azetidine-3,4′-pyrrolo[1,2-b]pyrazole]-1-carboxamide
1-{(3R)-2′-[6-amino-5-(trifluoromethyl) pyridin-3-yl]-5′,6′-dihydrospiro[pyrrolidine-3,4′-pyrrolo[1,2-b]pyrazol]-1-yl}-2-(methylamino)ethan-1-one
(2R)-1-{(3R)-2′-[6-amino-5-(trifluoromethyl)pyridin-3-yl]-5′,6′-dihydrospiro[pyrrolidine-3,4′-pyrrolo[1,2-b]pyrazol]-1-yl}-2-(methylamino)propan-1-one
(5R)-5-({(3R)-2′-[6-amino-5-(trifluoromethyl)pyridin-3-yl]-5′,6′-dihydrospiro[pyrrolidine-3,4′-pyrrolo[1,2-b]pyrazol]-1-yl}methyl)pyrrolidin-2-one
5-{(3R)-1-[(1,3,4-thiadiazol-2-yl)methyl]-5′,6′-dihydrospiro[pyrrolidine-3,4′-pyrrolo[1,2-b]pyrazol]-2′-yl}-3-(trifluoromethyl)pyridin-2-amine
(5S)-5-({(3R)-2′-[6-amino-5-(trifluoromethyl)pyridin-3-yl]-5′,6′-dihydrospiro[pyrrolidine-3,4′-pyrrolo[1,2-b]pyrazol]-1-yl}methyl)pyrrolidin-2-one
5-{(3′R)-1′-[1-(6-methoxypyridin-2-yl)ethyl]-6,7-dihydrospiro[pyrazolo[5,1-c][1,4]oxazine-4,3′-pyrrolidin]-2-yl}-3-(trifluoromethyl)pyridin-2-amine (diastereomer 1 and diastereomer 2)
5-{(3R)-1-[cyclopropyl(6-methoxypyridin-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3′R)-1′-[cyclopropyl(6-methoxypyridin-2-yl)methyl]-6,7-dihydrospiro[pyrazolo[5,1-c][1,4]oxazine-4,3′-pyrrolidin]-2-yl}-3-(trifluoromethyl)pyridin-2-amine (diastereomer 1 and diastereomer 2)
5-{(3R)-1-[1-(3-fluoropyridin-2-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
3-(difluoromethoxy)-5-{(3′R)-1′-[1-(1H-imidazol-2-yl)propyl]-6,7-dihydrospiro[pyrazolo[5,1-c][1,4]oxazine-4,3′-pyrrolidin]-2-yl}pyridin-2-amine (diastereomer 1 and diastereomer 2)
3-(difluoromethoxy)-5-{(3′R)-1′-[1-(1H-imidazol-2-yl)propyl]-6,7-dihydrospiro[pyrazolo[5,1-c][1,4]oxazine-4,3′-pyrrolidin]-2-yl}pyridin-2-amine (diastereomer 1)
3-(difluoromethoxy)-5-{(3′R)-1′-[1-(1H-imidazol-2-yl)propyl]-6,7-dihydrospiro[pyrazolo[5,1-c][1,4]oxazine-4,3′-pyrrolidin]-2-yl}pyridin-2-amine (diastereomer 2)
2-amino-5-{(3′R)-1′-[1-(1H-imidazol-2-yl)propyl]-6,7-dihydrospiro[pyrazolo[5,1-c][1,4]oxazine-4,3′-pyrrolidin]-2-yl}pyridine-3-carbonitrile (diastereomer 1 and diastereomer 2)
2-amino-5-{(3′R)-1′-[1-(1H-imidazol-2-yl)propyl]-6,7-dihydrospiro[pyrazolo[5,1-c][1,4]oxazine-4,3′-pyrrolidin]-2-yl}pyridine-3-carbonitrile (diastereomer 1)
2-amino-5-{(3′R)-1′-[1-(1H-imidazol-2-yl)propyl]-6,7-dihydrospiro[pyrazolo[5,1-c][1,4]oxazine-4,3′-pyrrolidin]-2-yl}pyridine-3-carbonitrile (diastereomer 2)
3-(difluoromethyl)-5-{(3′R)-1′-[1-(1H-imidazol-2-yl)propyl]-6,7-dihydrospiro[pyrazolo[5,1-c][1,4]oxazine-4,3′-pyrrolidin]-2-yl}pyridin-2-amine (diastereomer 1 and diastereomer 2)
3-(difluoromethyl)-5-{(3′R)-1′-[1-(1H-imidazol-2-yl)propyl]-6,7-dihydrospiro[pyrazolo[5,1-c][1,4]oxazine-4,3′-pyrrolidin]-2-yl}pyridin-2-amine (diastereomer 1)
3-(difluoromethyl)-5-{(3′R)-1′-[1-(1H-imidazol-2-yl)propyl]-6,7-dihydrospiro[pyrazolo[5,1-c][1,4]oxazine-4,3′-pyrrolidin]-2-yl}pyridin-2-amine (diastereomer 2)
(3R)-2′-(6-amino-5-{[1-(1-propyl-1H-pyrazol-5-yl)ethyl]oxy}pyridin-3-yl)-N-ethyl-5′,6′-dihydrospiro[pyrrolidine-3,4′-pyrrolo[1,2-b]pyrazole]-1-carboxamide (diastereomer 1 and diastereomer 2)
(3R)-2′-(6-amino-5-{[1-(1,3-thiazol-4-yl)ethyl]oxy}pyridin-3-yl)-N-ethyl-5′,6′-dihydrospiro[pyrrolidine-3,4′-pyrrolo[1,2-b]pyrazole]-1-carboxamide (diastereomer 1 and diastereomer 2)
(3R)-2′-(6-amino-5-{[1-(pyrimidin-4-yl)ethyl]oxy}pyridin-3-yl)-N-(propan-2-yl)-5′,6′-dihydrospiro[pyrrolidine-3,4′-pyrrolo[1,2-b]pyrazole]-1-carboxamide (diastereomer 1 and diastereomer 2)
(3R)-2′-(6-amino-5-{[1-(2,4-dimethylpyrimidin-5-yl)ethyl]oxy}pyridin-3-yl)-N-(propan-2-yl)-5′,6′-dihydrospiro[pyrrolidine-3,4′-pyrrolo[1,2-b]pyrazole]-1-carboxamide (diastereomer 1)
(3R)-2′-(6-amino-5-{[1-(2,4-dimethylpyrimidin-5-yl)ethyl]oxy}pyridin-3-yl)-N-(propan-2-yl)-5′,6′-dihydrospiro[pyrrolidine-3,4′-pyrrolo[1,2-b]pyrazole]-1-carboxamide (diastereomer 2)
(3R)-2′-{6-amino-5-[(1R)-1-(5-methyl-1,3,4-oxadiazol-2-yl)ethoxy]pyridin-3-yl}-N-(propan-2-yl)-5′,6′-dihydrospiro[pyrrolidine-3,4′-pyrrolo[1,2-b]pyrazole]-1-carboxamide
(3R)-2′-{6-amino-5-[(1R)-1-(1-methyl-1H-1,2,3-triazol-5-yl)ethoxy]pyridin-3-yl}-N-(propan-2-yl)-5′,6′-dihydrospiro[pyrrolidine-3,4′-pyrrolo[1,2-b]pyrazole]-1-carboxamide
(3R)-2′-(6-amino-5-{[1-(1-methyl-1H-1,2,3-triazol-4-yl)ethyl]oxy}pyridin-3-yl)-N-(propan-2-yl)-5′,6′-dihydrospiro[pyrrolidine-3,4′-pyrrolo[1,2-b]pyrazole]-1-carboxamide (diastereomer 1 and diastereomer 2)
(5S)-5-{[(3R)-2′-{6-amino-5-[(1R)-1-phenylethoxy]pyridin-3-yl}-5′,6′-dihydrospiro[pyrrolidine-3,4′-pyrrolo[1,2-b]pyrazol]-1-yl]methyl}pyrrolidin-2-one
(5R)-5-{[(3R)-2′-{6-amino-5-[(1R)-1-phenylethoxy]pyridin-3-yl}-5′,6′-dihydrospiro[pyrrolidine-3,4′-pyrrolo[1,2-b]pyrazol]-1-yl]methyl}pyrrolidin-2-one
(3R)-2′-(6-amino-5-{1-[4-(methylsulfonyl)phenyl]ethoxy}pyridin-3-yl)-N-isopropyl-5′,6′-dihydro-1H-spiro[pyrrolidine-3,4′-pyrrolo[1,2-b]pyrazole]-1-carboxamide (diastereomer 1 and diastereomer 2)
(3R)-2′-{6-amino-5-[(1R)-1-(2-methyl-1,3-thiazol-4-yl)ethoxy]pyridin-3-yl}-N-(propan-2-yl)-5′,6′-dihydrospiro[pyrrolidine-3,4′-pyrrolo[1,2-b]pyrazole]-1-carboxamide
(3R)-2′-[6-amino-5-({1-[3-(methanesulfonyl)phenyl]ethyl}oxy)pyridin-3-yl]-N-(propan-2-yl)-5′,6′-dihydrospiro[pyrrolidine-3,4′-pyrrolo[1,2-b]pyrazole]-1-carboxamide (diastereomer 1 and diastereomer 2)
(3R)-2′-(6-amino-5-{[1-(4-methyl-1,3-thiazol-2-yl)ethyl]oxy}pyridin-3-yl)-N-ethyl-5′,6′-dihydrospiro[pyrrolidine-3,4′-pyrrolo[1,2-b]pyrazole]-1-carboxamide (diastereomer 1 and diastereomer 2)
(3R)-2′-(6-amino-5-{[1-(4,5-dimethyl-1,3-thiazol-2-yl)ethyl]oxy}pyridin-3-yl)-N-ethyl-5′,6′-dihydrospiro[pyrrolidine-3,4′-pyrrolo[1,2-b]pyrazole]-1-carboxamide (diastereomer 1 and diastereomer 2)
(3R)-2′-(6-amino-5-{[1-(5-methyl-1,3-thiazol-2-yl)ethyl]oxy}pyridin-3-yl)-N-ethyl-5′,6′-dihydrospiro[pyrrolidine-3,4′-pyrrolo[1,2-b]pyrazole]-1-carboxamide (diastereomer 1 and diastereomer 2)
(3R)-2′-{6-amino-5-[(1R)-1-(pyridin-4-yl)ethoxy]pyridin-3-yl}-N-(propan-2-yl)-5′,6′-dihydrospiro[pyrrolidine-3,4′-pyrrolo[1,2-b]pyrazole]-1-carboxamide
(3R)-2′-{6-amino-5-[(1R)-1-(pyridin-4-yl)ethoxy]pyridin-3-yl}-N-cyclobutyl-5′,6′-dihydrospiro[pyrrolidine-3,4′-pyrrolo[1,2-b]pyrazole]-1-carboxamide
6-(1-{(3′R)-2-[6-amino-5-(trifluoromethyl)pyridin-3-yl]-6,7-dihydrospiro[pyrazolo[5,1-c][1,4]oxazine-4,3′-pyrrolidin]-1′-yl}ethyl)pyridin-2-ol (diastereomer 1 and diastereomer 2)
6-(1-{(3′R)-2-[6-amino-5-(trifluoromethyl)pyridin-3-yl]-6,7-dihydrospiro[pyrazolo[5,1-c][1,4]oxazine-4,3′-pyrrolidin]-1′-yl}ethyl)pyridin-2-ol (diastereomer 1)
6-(1-{(3′R)-2-[6-amino-5-(trifluoromethyl)pyridin-3-yl]-6,7-dihydrospiro[pyrazolo[5,1-c][1,4]oxazine-4,3′-pyrrolidin]-1′-yl}ethyl)pyridin-2-ol (diastereomer 2)
6-({(3R)-2′-[6-amino-5-(trifluoromethyl) pyridin-3-yl]-5′,6′-dihydrospiro[pyrrolidine-3,4′-pyrrolo[1,2-b]pyrazol]-1-yl}(cyclopropyl)methyl)pyridin-2-ol (diastereomer 1 and diastereomer 2)
6-({(3′R)-2-[6-amino-5-(trifluoromethyl)pyridin-3-yl]-6,7-dihydrospiro[pyrazolo[5,1-c][1,4]oxazine-4,3′-pyrrolidin]-1′-yl}(cyclopropyl)methyl)pyridin-2-ol (diastereomer 1 and diastereomer 2)
5-{(3R)-1-[cyclopropyl(1H-1,2,4-triazol-5-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[cyclopropyl(1H-1,2,4-triazol-5-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3R)-1-[(3,3-difluorocyclobutyl)(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[(3,3-difluorocyclobutyl)(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[(3,3-difluorocyclobutyl)(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{1′-[(3,3-difluorocyclobutyl)(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine (isomer 1)
5-{1′-[(3,3-difluorocyclobutyl)(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine (isomer 2)
5-{1′-[(3,3-difluorocyclobutyl)(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine (isomer 3)
5-{1′-[(3,3-difluorocyclobutyl)(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine (isomer 4)
5-{(3R)-1-[1-(1H-pyrazol-3-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[(1H-imidazol-2-yl)(1-methylcyclopropyl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[(1H-imidazol-2-yl)(1-methylcyclopropyl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[(1H-imidazol-2-yl)(1-methylcyclopropyl)methyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3R)-1-[cyclobutyl(4H-1,2,4-triazol-3-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[cyclobutyl(4H-1,2,4-triazol-3-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[cyclobutyl(4H-1,2,4-triazol-3-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3′R)-1′-[cyclohexyl(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine (diastereomer 1 and diastereomer 2)
5-{(3R)-1-[cyclohexyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[1-(4-chloro-1H-imidazol-2-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[(3,3-difluorocyclobutyl)(4H-1,2,4-triazol-3-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}-3-(trifluoromethyl)pyrazin-2-amine (diastereomer 1 and diastereomer 2)
5-{(3R)-1-[(1H-imidazol-2-yl)(1-methylcyclobutyl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[1-(1H-pyrazol-3-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[1-(1H-pyrazol-3-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3R)-1-[cyclopropyl(1H-1,2,3-triazol-5-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[cyclopropyl(1H-1,2,4-triazol-5-yl)methyl]-5′,6′-dihydrospiro[pyrrolidine-3,4′-pyrrolo[1,2-b]pyrazol]-2′-yl}-3-[(1R)-1-(2,6-difluorophenyl)ethoxy]pyridin-2-amine (diastereomer 1 and diastereomer 2)
5-{(3R)-1-[(1-methylcyclobutyl)(4H-1,2,4-triazol-3-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[1-(4-chloro-1H-imidazol-2-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[1-(4-chloro-1H-imidazol-2-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3R)-1-[cyclopropyl(1H-1,2,4-triazol-5-yl)methyl]-5′,6′-dihydrospiro[pyrrolidine-3,4′-pyrrolo[1,2-b]pyrazol]-2′-yl}-3-[(1R)-1-(3-fluoropyridin-2-yl)ethoxy]pyridin-2-amine (diastereomer 1 and diastereomer 2)
5-{(3R)-1-[(3,3-difluorocyclobutyl)(4H-1,2,4-triazol-3-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[(3,3-difluorocyclobutyl)(4H-1,2,4-triazol-3-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3R)-1-[cyclopentyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3′R)-1′-[cyclopropyl(1H-1,2,3-triazol-5-yl)methyl]-6,7-dihydrospiro[pyrazolo[5,1-c][1,4]oxazine-4,3′-pyrrolidin]-2-yl}-3-(trifluoromethyl)pyridin-2-amine (diastereomer 1 and diastereomer 2)
5-{(3R)-1-[cyclopropyl(1-methyl-1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3R)-1-[1-(4H-1,2,4-triazol-3-yl)(1-²H)propyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[1-(4H-1,2,4-triazol-3-yl)(1-²H)propyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3R)-1-[1-(4H-1,2,4-triazol-3-yl)(1-²H)propyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3′R)-1′-[1-(4H-1,2,4-triazol-3-yl)(1-²H)propyl]-6,7-dihydrospiro[pyrazolo[5,1-c][1,4]oxazine-4,3′-pyrrolidin]-2-yl}-3-(trifluoromethyl)pyridin-2-amine (diastereomer 1 and diastereomer 2)
5-{(3′R)-1′-[cyclopropyl(1H-imidazol-2-yl)(²H)methyl]-6,7-dihydrospiro[pyrazolo[5,1-c][1,4]oxazine-4,3′-pyrrolidin]-2-yl}-3-(trifluoromethyl)pyridin-2-amine (diastereomer 1 and diastereomer 2)
5-{(3′R)-1′-[cyclopropyl(1H-imidazol-2-yl)(²H)methyl]-6,7-dihydrospiro[pyrazolo[5,1-c][1,4]oxazine-4,3′-pyrrolidin]-2-yl}-3-(trifluoromethyl)pyridin-2-amine (diastereomer 1)
5-{(3′R)-1′-[cyclopropyl(1H-imidazol-2-yl)(²H)methyl]-6,7-dihydrospiro[pyrazolo[5,1-c][1,4]oxazine-4,3′-pyrrolidin]-2-yl}-3-(trifluoromethyl)pyridin-2-amine (diastereomer 2)
5-{(3′R)-1′-[1-(1H-imidazol-2-yl)(1-²H)propyl]-6,7-dihydrospiro[pyrazolo[5,1-c][1,4]oxazine-4,3′-pyrrolidin]-2-yl}-3-(trifluoromethyl)pyridin-2-amine (diastereomer 1 and diastereomer 2)
5-{(3′R)-1′-[1-(1H-imidazol-2-yl)(1-²H)propyl]-6,7-dihydrospiro[pyrazolo[5,1-c][1,4]oxazine-4,3′-pyrrolidin]-2-yl}-3-(trifluoromethyl)pyridin-2-amine (diastereomer 1)
5-{(3′R)-1′-[1-(1H-imidazol-2-yl)(1-²H)propyl]-6,7-dihydrospiro[pyrazolo[5,1-c][1,4]oxazine-4,3′-pyrrolidin]-2-yl}-3-(trifluoromethyl)pyridin-2-amine (diastereomer 2)
5-{(3R)-1-[1-(1H-imidazol-2-yl)(1-²H)propyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[1-(1H-imidazol-2-yl)(1-²H)propyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[1-(1H-imidazol-2-yl)(1-²H)propyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3R)-1-[cyclopropyl(1H-imidazol-2-yl)(2H)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[cyclopropyl(1H-imidazol-2-yl)(2H)methyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[cyclopropyl(1H-imidazol-2-yl)(²H)methyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3R)-1-[cyclopropyl(2-methoxypyridin-3-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
3-[{(3R)-2′-[6-amino-5-(trifluoromethyl)pyridin-3-yl]-5′,6′-dihydro-1H-spiro[pyrrolidine-3,4′-pyrrolo[1,2-b]pyrazol]-1-yl}(cyclopropyl)methyl]pyridin-2(1H)-one (diastereomer 1 and diastereomer
2)
5-{(3R)-1-[(1-fluorocyclopropyl)(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[(1-fluorocyclopropyl)(1H-imidazol-2-yl)methyl]-5′,6′-dihydrospiro[pyrroidine-3,4′-pyrrolo[1,2-b]pyrazol]-2′-yl}-3-(trifluoromethyl)pyridin-2-amine (diastereomer 1)
5-{(3R)-1-[(1-fluorocyclopropyl)(1H-imidazol-2-yl)methyl]-5′,6′-dihydrospiro[pyrrolidine-3,4′-pyrrolo[1,2-b]pyrazol]-2′-yl}-3-(trifluoromethyl) pyridin-2-amine (diastereomer 2)
5-{(3R)-1-[cyclopropyl(4-methoxypyrimid in-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[cyclobutyl(1H-1,2,4-triazol-5-yl)methyl]-5′,6′-dihydrospiro[pyrrolidine-3,4′-pyrrolo[1,2-b]pyrazol]-2′-yl}-3-(trifluoromethoxy)pyridin-2-amine (diastereomer 1 and diastereomer 2)
5-{(3R)-1-[cyclopropyl(1H-1,2,4-triazol-5-yl)methyl]-5′,6′-dihydrospiro[pyrrolidine-3,4′-pyrrolo[1,2-b]pyrazol]-2′-yl}-3-(trifluoromethoxy)pyridin-2-amine (diastereomer 1 and diastereomer 2)
5-{(3′R)-1′-[cyclopropyl(1H-1,2,3-triazol-5-yl)methyl]-6,7-dihydrospiro[pyrazolo[5,1-c][1,4]oxazine-4,3′-pyrrolidin]-2-yl}-3-(trifluoromethyl)pyridin-2-amine (diastereomer 1)
5-{(3′R)-1′-[cyclopropyl(1H-1,2,3-triazol-5-yl)methyl]-6,7-dihydrospiro[pyrazolo[5,1-c][1,4]oxazine-4,3′-pyrrolidin]-2-yl}-3-(trifluoromethyl)pyridin-2-amine (diastereomer 2)
5-{(3R)-1-[(1-fluorocyclobutyl)(4H-1,2,4-triazol-3-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[cyclopropyl(pyrimidin-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[cyclopropyl(1H-1,2,4-triazol-5-yl)methyl]-5′,6′-dihydrospiro[pyrrolidine-3,4′-pyrrolo[1,2-b]pyrazol]-2′-yl}-3-[(1R)-1-(2,6-difluorophenyl)ethoxy]pyridin-2-amine (diastereomer 1)
5-{(3R)-1-[cyclopropyl(1H-1,2,4-triazol-5-yl)methyl]-5′,6′-dihydrospiro[pyrrolidine-3,4′-pyrrolo[1,2-b]pyrazol]-2′-yl}-3-[(1R)-1-(2,6-difluorophenyl)ethoxy]pyridin-2-amine (diastereomer 2)
5-{(3R)-1-[cyclopropyl(1H-1,2,4-triazol-5-yl)methyl]-5′,6′-dihydrospiro[pyrrolidine-3,4′-pyrrolo[1,2-b]pyrazol]-2′-yl}-3-[(1R)-1-(3-fluoropyridin-2-yl)ethoxy]pyridin-2-amine (diastereomer 1)
5-{(3R)-1-[cyclopropyl(1H-1,2,4-triazol-5-yl)methyl]-5′,6′-dihydrospiro[pyrrolidine-3,4′-pyrrolo[1,2-b]pyrazol]-2′-yl}-3-[(1R)-1-(3-fluoropyridin-2-yl)ethoxy]pyridin-2-amine (diastereomer 2)
5-{(3′R)-1′-[cyclobutyl(4H-1,2,4-triazol-3-yl)methyl]-6,7-dihydrospiro[pyrazolo[5,1-c][1,4]oxazine-4,3′-pyrrolidin]-2-yl}-3-(trifluoromethyl)pyridin-2-amine (diastereomer 1)
5-{(3R)-1-[cyclopentyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[cyclopentyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3R)-1-[(4,4-difluorocyclohexyl)(1H-1,2,4-triazol-5-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[(4,4-difluorocyclohexyl)(1H-1,2,4-triazol-5-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[(4,4-difluorocyclohexyl)(1H-1,2,4-triazol-5-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3R)-1-[2,2-difluoro-1-(1H-imidazol-2-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[cyclopropyl(1H-1,2,3-triazol-5-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[cyclopropyl(1H-1,2,3-triazol-5-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3′R)-1′-[cyclobutyl(4H-1,2,4-triazol-3-yl)methyl]-6,7-dihydrospiro[pyrazolo[5,1-c][1,4]oxazine-4,3′-pyrrolidin]-2-yl}-3-(trifluoromethyl)pyridin-2-amine (diastereomer 1 and diastereomer 2)
5-{(3R)-1-[cyclopropyl(1H-1,2,4-triazol-5-yl)methyl]-5′,6′-dihydrospiro[pyrrolidine-3,4′-pyrrolo[1,2-b]pyrazol]-2′-yl}-3-(trifluoromethoxy)pyridin-2-amine (diastereomer 1)
5-{(3R)-1-[cyclopropyl(1H-1,2,4-triazol-5-yl)methyl]-5′,6′-dihydrospiro[pyrrolidine-3,4′-pyrrolo[1,2-b]pyrazol]-2′-yl}-3-(trifluoromethoxy)pyridin-2-amine (diastereomer 2)
(3R)-2′-{5-amino-6-[(1R)-1-phenylethoxy]pyrazin-2-yl}-N-ethyl-5′,6′-dihydrospiro[pyrrolidine-3,4′-pyrrolo[1,2-b]pyrazole]-1-carboxamide
5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyrazin-2-amine (diastereomer 1 and diastereomer 2)
5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyrazin-2-amine (diastereomer 1)
5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyrazin-2-amine (diastereomer 2)

or a stereoisomer, a tautomer, an N-oxide, a hydrate, a solvate, or a salt thereof, or a mixture of same.

### Embodiment 10

A compound of general formula (I) according to any one of embodiments 1, 2, 3, 4, 5, 6, 7, 8 or 9 for the use as a medicament.

### Embodiment 11

A compound of general formula (I) according to any one of embodiments 1, 2, 3, 4, 5, 6, 7, 8 or 9 for use in the treatment or prophylaxis of a disease.

### Embodiment 12

A pharmaceutical composition comprising a compound of general formula (I) according to any one of embodiments 1, 2, 3, 4, 5, 6, 7, 8 or 9 and one or more pharmaceutically acceptable excipients.

### Embodiment 13

A pharmaceutical combination comprising:
one or more first active ingredients, in particular compounds of general formula (I) according to any one of embodiments 1, 2, 3, 4, 5, 6, 7, 8 or 9 and
one or more pharmaceutical active anti cancer compounds or
one or more pharmaceutical active immune checkpoint inhibitors.

### Embodiment 14

A pharmaceutical combination according to claim 10, characterized in that the pharmaceutical active immune checkpoint inhibitor is an antibody.

### Embodiment 15

Use of a compound of general formula (I) according to any one of embodiments 1, 2, 3, 4, 5, 6, 7, 8 or 9 for the treatment or prophylaxis of a disease.

### Embodiment 16

Use of a compound of general formula (I) according to any one of embodiments 1, 2, 3, 4, 5, 6, 7, 8 or 9 for the preparation of a medicament for the treatment or prophylaxis of a disease.

### Embodiment 17

Use according to embodiments 15 or 16, wherein the disease is cancer or conditions with dysregulated immune responses or other disorders associated with aberrant MAP4K1 signaling, such as liquid and solid tumours.

### Embodiment 18

Use according to embodiments 15 or 16, wherein the diseases, respectively the disorders are benign hyperplasias, atherosclerotic disorders, sepsis, autoimmune disorders, vascular disorders, viral infections, neurodegenerative disorders, in inflammatory disorders, and male fertility control.

This invention also covers the following embodiments:

### Embodiment A′

### Compounds of Formula (I′)

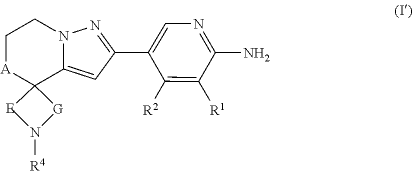 [1310] A is selected from a single bond, —CH₂— or —O—;
E is —(CH₂)ₙ— with n=1 or 2;
G is —(CH₂)ₘ— with m=1, 2 or 3 and with the condition, that the cyclic substructure comprising E-N-G is a 4 to 6 membered heterocycloalkyl;
R¹ is selected from
   —NO₂; —CN;
   C₁₋₄-alkyl optionally substituted by —OH or 1 to 3-F;
   3 to 6 membered heterocycloalkyl with 1-2 nitrogen atoms; phenyl optionally substituted by halogen, —CN, C₁₋₄-alkyl, C₁₋₄-haloalkyl, C₃₋₆-cycloalkyl, O—C₁₋₄-alkyl, O—C₃₋₆-cycloalky, C₁₋₃-alkinyl, SR_{d}, SOR_{d}, SO₂R_{d}, SONR_{d} or SON(H)R_{d} wherein R_{d} is selected from methyl, ethyl, cyclopropyl, isopropyl and tert·butyl;
   5 to 6 membered heteroaryl with 1 or 2 heteroatoms selected from N or S and optionally substituted by halogen, —CN, C₁₋₄-alkyl, C₁₋₄-haloalkyl, C₃₋₆-cycloalkyl, O—C₁₋₄-alkyl, O—C₃₋₆-cycloalky, C₁₋₃-alkinyl, SR_{d}, SOR_{d}, SO₂R_{d}, SONR_{d} or SON(H)R_{d} wherein R_{d} is selected from methyl, ethyl, cyclopropyl, isopropyl and tert·butyl; —O—CH₃; —O—CH(CH₃)₂; —O—CHF₂; —O—CF₃;

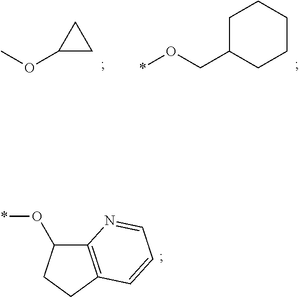

—O—CHRₐ-phenyl optionally substituted by one or two halogen, —CN, C₁₋₄-alkyl or —SO₂—CH₃ and with Rₐ selected from —H or —CH₃, —CH₂—CH₃, —CH(CH₃)₂ or -cyclopropyl;
—O—CHRₐ-6 membered heteroaryl optionally substituted one or two halogen, —CN, C₁₋₄-alkyl or —SO₂—CH₃ and with Rₐ selected from —H or —CH₃, —CH₂—CH₃, —CH(CH₃)₂ or -cyclopropyl;
COORₐ with Rₐ selected from —H or —CH₃;
C(═O)-methyl, optionally substituted by 1 to 3-F;
—C(═O)—NR_{b}R_{c} wherein R_{b} is selected from —H, —CH₃ or —CH₂—CH₃ and wherein R_{c} is selected from —H, —CH₃, —CH₂—CH₃;
S—CR_{f}R_{g}Rₕ, wherein R_{f} is selected from —H or —F, wherein R_{g} is selected from —H, —F or —CH₃ and wherein Rₕ is selected from C₅₋₆-cycloalkyl or phenyl;

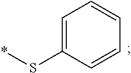 [SMILES: *Sc1ccccc1]

R² is selected from —H, —CH₃ or —F; or
R⁴ is selected from
C₁₋₄-alkyl or C₃₋₆-cycloalkyl, both optionally substituted one or more times by
   —F, —Cl, —Br, —CN;
   —OH, —ORᵢ wherein Rᵢ is selected from C₁₋₄-alkyl or C₃₋₆Cycloalkyl), —CFₓH_{y} with x and y=0, 1, 2 or 3 and x+y=3;
   —NRⱼRₖ wherein Rⱼ and Rₖ are independently selected from —H and C₁-C₄-alkyl or
   phenyl or 5 to 6 membered heteroaryl, both optionally substituted one or more times by
      halogen, —CN,
      —CFₓH_{y} with x and y=0, 1, 2 or 3 and x+y=3,
      C₁₋₄-alkyl optionally substituted with one or more halogen;
      C₃₋₆-cycloalkyl;
      O—C₁₋₄-alkyl,
      O—C₃₋₆-cycloalky,
      C₁₋₃-alkinyl,
      NRⱼRₖ wherein Rⱼ and Rₖ are independently selected from —H and C₁-C₄-alkyl
      SR_{d}, SOR_{d}, SO₂R_{d}, SONR_{d} or SON(H)R_{d} wherein R_{d} is selected from methyl, ethyl, cyclopropyl, isopropyl and tert·butyl;
   C(═O)—Rᵢ in which Rᵢ is selected from
      C₁₋₄-alkyl or C₃₋₆-cycloalkyl, both optionally substituted one or more times by
         —F, —Cl, —Br, —CN;
         —OH, —ORᵢ with Rᵢ selected from C₁₋₄-alkyl or C₃₋₆Cycloalkyl), —CFₓH_{y} with x and y=0, 1, 2 or 3 and x+y=3;
         NRⱼRₖ wherein Rⱼ and Rₖ are independently selected from —H and C₁-C₄-alkyl
         phenyl or 5 to 6 membered heteroaryl, both optionally substituted one or more times by
         halogen, —CN,
         —CFₓH_{y} with x and y=0, 1, 2 or 3 and x+y=3,
         C₁₋₄-alkyl optionally substituted with one or more halogen;
         C₃₋₆-cycloalkyl;
         O—C₁₋₄-alkyl,
         O—C₃₋₆-cycloalky,
         C₁₋₃-alkinyl,
         —NRⱼRₖ wherein Rⱼ and Rₖ are independently selected from —H and C₁-C₄-alkyl
         SR_{d}, SOR_{d}, SO₂R_{d}, SONR_{d} or SON(H)R_{d} wherein R_{d} is selected from methyl, ethyl, cyclopropyl, isopropyl and tert·butyl;
      phenyl or 5 to 6 membered heteroaryl, both optionally substituted one or more times by
         halogen, —CN,
         —CFₓH_{y} with x and y=0, 1, 2 or 3 and x+y=3,
         C₁₋₄-alkyl optionally substituted with one or more halogen;
         C₃₋₆-cycloalkyl;
         O—C₁₋₄-alkyl,
         O—C₃₋₆-cycloalky,
         C₁₋₃-alkinyl,
         NRⱼRₖ wherein Rⱼ and Rₖ are independently selected from —H and C₁-C₄-alkyl
         SR_{d}, SOR_{d}, SO₂R_{d}, SONR_{d} or SON(H)R_{d} wherein R_{d} is selected from methyl, ethyl, cyclopropyl, isopropyl and tert·butyl;
   C(═O)—NRₒRₚ wherein Rₒ and Rₚ are independently selected from
      H
      C₁₋₄-alkyl or C₃₋₆-cycloalkyl, both optionally substituted one or more times by
         —F, —Cl, —Br, —CN;
         —OH, —ORᵢ with Rᵢ selected from C₁₋₄-alkyl or C₃₋₆Cycloalkyl), —CFₓH_{y} with x and y=0, 1, 2 or 3 and x+y=3;
         —NRⱼRₖ wherein Rⱼ and Rₖ are independently selected from —H and C₁-C₄-alkyl
         phenyl or 5 to 6 membered heteroaryl, both optionally substituted one or more times by
         halogen, —CN,
         —CFₓH_{y} with x and y=0, 1, 2 or 3 and x+y=3,
         C₁₋₄-alkyl optionally substituted with one or more halogen;
         C₃₋₆-cycloalkyl;
         O—C₁₋₄-alkyl,
         O—C₃₋₆-cycloalky,
         C₁₋₃-alkinyl,
         —NRⱼRₖ wherein Rⱼ and Rₖ are independently selected from —H and C₁-C₄-alkyl
         SR_{d}, SOR_{d}, SO₂R_{d}, SONR_{d} or SON(H)R_{d} wherein R_{d} is selected from methyl, ethyl, cyclopropyl, isopropyl and tert·butyl;
      phenyl or 5 to 6 membered heteroaryl, both optionally substituted one or more times by
         halogen, —CN,
         —CFₓH_{y} with x and y=0, 1, 2 or 3 and x+y=3,
         C₁₋₄-alkyl optionally substituted with one or more halogen;
         C₃₋₆-cycloalkyl;
         O—C₁₋₄-alkyl,
         O—C₃₋₆-cycloalky,
         C₁₋₃-alkinyl,
         —NRⱼRₖ wherein Rⱼ and Rₖ are independently selected from —H and C₁-C₄-alkyl
         SR_{d}, SOR_{d}, SO₂R_{d}, SONR_{d} or SON(H)R_{d} wherein R_{d} is selected from methyl, ethyl, cyclopropyl, isopropyl and tert·butyl;

or a stereoisomer, a tautomer, an N-oxide, a hydrate, a solvate, or a salt thereof, or a mixture of same.

### Embodiment A″

### Compounds of Formula (I″)

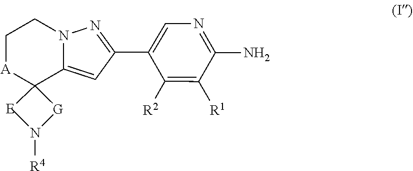 [1375] wherein
A is selected from a single bond, —CH₂— or —O—;
E is —(CH₂)ₙ— with n=1 or 2;
G is —(CH₂)— and with the condition, that the cyclic substructure comprising E-N-G is a 4 to 5 membered heterocycloalkyl;
R¹ is selected from
   —NO₂; —CN;
   C₁₋₄-alkyl optionally substituted by —OH or 1 to 3-F;
   cyclopropyl;
   4 to 6 membered heterocycloalkyl or heterocycloalkenyl, both with 1-2 nitrogen atoms, but always linked via a carbon atom to the phenyl group of compounds of formula (I) and both optionally substituted by C₁₋₄-alkyl;
   phenyl optionally substituted by halogen, —CN, C₁₋₄-alkyl, C₁₋₄-haloalkyl, C₃₋₆-cycloalkyl, O—C₁₋₄-alkyl, O—C₃₋₆-cycloalky, C₁₋₃-alkinyl, —C(O)—R_{d}, SR_{d}, SOR_{d}, SO₂R_{d}, SONR_{d}, SON(H)R_{d}, —SO₂—NH₂, —SO₂—N(H)—Rₐ or —SO₂—N(R_{d})₂, wherein R_{d} is selected from methyl, ethyl, cyclopropyl, isopropyl and tert·butyl;
   5 to 6 membered heteroaryl with 1 or 2 heteroatoms selected from N or S and optionally substituted by halogen, —CN, C₁₋₄-alkyl, C₁₋₄-haloalkyl, C₃₋₆-cycloalkyl, O—C₁₋₄-alkyl, O—C₃₋₆-cycloalkyl, C₁₋₃-alkinyl, SR_{d}, SOR_{d}, SO₂R_{d}, SONR_{d} or SON(H)R_{d} wherein R_{d} is selected from methyl, ethyl, cyclopropyl, isopropyl and tert·butyl;
   —O—CH₃; —O—CH(CH₃)₂; —O—CHF₂; —O—CF₃;

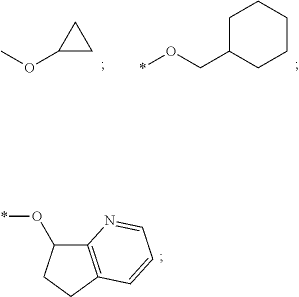

—O—CHRₐ-phenyl optionally substituted by one or two halogen, —CN, C₁₋₄-alkyl or —SO₂—CH₃ and with Rₐ selected from —H or —CH₃, —CH₂—CH₃, —CH(CH₃)₂ or -cyclopropyl;
—O—CHRₐ-6 membered heteroaryl optionally substituted one or two halogen, —CN, C₁₋₄-alkyl or —SO₂—CH₃ and with Rₐ selected from —H or —CH₃, —CH₂—CH₃, —CH(CH₃)₂ or -cyclopropyl;
—COORₐ with Rₐ selected from —H or —CH₃;
C(═O)-methyl, optionally substituted by 1 to 3-F;
—C(═O)—NR_{b}R_{c} wherein R_{b} is selected from —H, —CH₃ or —CH₂—CH₃ and wherein R_{c} is selected from —H, —CH₃, —CH₂—CH₃, cyclohexyl, phenyl or 5 to 6 membered heteroaryl, all optionally substituted by one or two halogen, —CN, —SO₂—CH₃ or C₁₋₄-alkyl, optionally substituted one or more times with halogen;
—S—CR_{f}R_{g}Rₕ, wherein R_{f} is selected from —H or —F, wherein R_{g} is selected from —H, —F or —CH₃ and wherein Rₕ is selected from H, F, —CH₃ or C₅₋₆-cycloalkyl or phenyl;

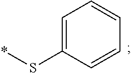 [SMILES: *Sc1ccccc1]

R² is selected from —H; or
R⁴ is selected from
   C₁₋₅-alkyl or C₃₋₆-cycloalkyl, both optionally substituted one or independently more times by
      —F, —Cl, —Br, —CN;
      —OH, —ORᵢ wherein Rᵢ is selected from C₁₋₄-alkyl or C₃₋₆Cycloalkyl),
      —CF₃, —CHF₂, —CH₂F, or —CH₂—CF₃;
      —NRⱼRₖ wherein Rⱼ and Rₖ are independently selected from —H and C₁-C₄-alkyl,
      —CH₂—O—CH₃ or
      C₃₋₄ cycloalkyl or 4 membered heterocycloalkyl, both optionally substituted by —F or —CH₃;
      phenyl or 5 to 6 membered heteroaryl, both optionally substituted one or more times by
         halogen, —CN,
         —CF₃, —CHF₂, —CH₂F,
         C₁₋₄-alkyl optionally substituted with one or more halogen;
         C₃₋₆-cycloalkyl;
         O—C₁₋₄-alkyl,
         O—C₃₋₆-cycloalky,
         C₁₋₃-alkinyl,
         —NRⱼRₖ wherein Rⱼ and Rₖ are independently selected from —H and C₁-C₄-alkyl
         SR_{d}, SOR_{d}, SO₂R_{d}, SONR_{d} or SON(H)R_{d} wherein R_{d} is selected from methyl, ethyl, cyclopropyl, isopropyl and tert·butyl;
   C(═O)—Rᵢ in which Rᵢ is selected from
      C₁₋₄-alkyl, C₃₋₆-cycloalkyl or C₃₋₆-heterocycloalkyl, both optionally substituted one or more times by
         —F, —Cl, —Br, —CN;
         —OH, —ORᵢ with Rᵢ selected from C₁₋₄-alkyl or C₃₋₆Cycloalkyl), —CF₃, —CHF₂, —CH₂F;
         —NRⱼRₖ wherein Rⱼ and Rₖ are independently selected from —H and C₁-C₄-alkyl
         phenyl or 5 to 6 membered heteroaryl, both optionally substituted one or more times by
         halogen, —CN,
         —CF₃, —CHF₂, —CH₂F,
         C₁₋₄-alkyl optionally substituted with one or more halogen;
         C₃₋₆-cycloalkyl;
         O—C₁₋₄-alkyl,
         O—C₃₋₆-cycloalky,
         C₁₋₃-alkinyl,
         —NRⱼRₖ wherein Rⱼ and Rₖ are independently selected from —H and C₁-C₄-alkyl
         SR_{d}, SOR_{d}, SO₂R_{d}, SONR_{d} or SON(H)R_{d} wherein R_{d} is selected from methyl, ethyl, cyclopropyl, isopropyl and tert·butyl;
      phenyl or 5 to 6 membered heteroaryl, both optionally substituted one or more times by
         halogen, —CN,
         —CF₃, —CHF₂, —CH₂F,
         C₁₋₄-alkyl optionally substituted with one or more halogen;
         C₃₋₆-cycloalkyl;
         O—C₁₋₄-alkyl,
         O—C₃₋₆-cycloalky,
         C₁₋₃-alkinyl,
         —NRⱼRₖ wherein Rⱼ and Rₖ are independently selected from —H and C₁-C₄-alkyl
         SR_{d}, SOR_{d}, SO₂R_{d}, SONR_{d} or SON(H)R_{d} wherein R_{d} is selected from methyl, ethyl, cyclopropyl, isopropyl and tert·butyl;
   C(═O)—NRₒRₚ wherein Rₒ and Rₚ are independently selected from
      H
      C₁₋₄-alkyl or C₃₋₆-cycloalkyl, both optionally substituted one or more times by
         —F, —Cl, —Br, —CN;
         —OH, —ORᵢ with Rᵢ selected from C₁₋₄-alkyl or C₃₋₆-cycloalkyl, —CF₃, —CHF₂, —CH₂F;
         —NRⱼRₖ wherein Rⱼ and Rₖ are independently selected from —H and C₁-C₄-alkyl
         phenyl or 5 to 6 membered heteroaryl, both optionally substituted one or more times by
         halogen, —CN,
         —CF₃, —CHF₂, —CH₂F,
         C₁₋₄-alkyl optionally substituted with one or more halogen;
         C₃₋₆-cycloalkyl;
         O—C₁₋₄-alkyl,
         O—C₃₋₆-cycloalky,
         C₁₋₃-alkinyl,
         —NRⱼRₖ wherein Rⱼ and Rₖ are independently selected from —H and C₁-C₄-alkyl
         SR_{d}, SOR_{d}, SO₂R_{d}, SONR_{d} or SON(H)R_{d} wherein R_{d} is selected from methyl, ethyl, cyclopropyl, isopropyl and tert·butyl;
      phenyl or 5 to 6 membered heteroaryl, both optionally substituted one or more times by
         halogen, —CN,
         —CF₃, —CHF₂, —CH₂F,
         C₁₋₄-alkyl optionally substituted with one or more halogen;
         C₃₋₆-cycloalkyl;
         O—C₁₋₄-alkyl,
         O—C₃₋₆-cycloalky,
         C₁₋₃-alkinyl,
         —NRⱼRₖ wherein Rⱼ and Rₖ are independently selected from —H and C₁-C₄-alkyl
         SR_{d}, SOR_{d}, SO₂R_{d}, SONR_{d} or SON(H)R_{d} wherein R_{d} is selected from methyl, ethyl, cyclopropyl, isopropyl and tert·butyl;

or a stereoisomer, a tautomer, an N-oxide, a hydrate, a solvate, or a salt thereof, or a mixture of same.

### Embodiment B

Of selected interest are compounds of formulas (I), (Ia), (I′) or (I″) (see embodiments 1, A, A′ and A″), wherein R₄ is

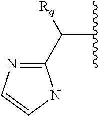

and R_{q} is selected from H, —CH₃, —CH₂—CH₃, —CH(CH₃)₂, —CH₂—O—CH₃, —CF₃, —CHF₂, —CH₂F, —CH₂—CF₃, cyclopropyl or cyclobutyl or a stereoisomer, a tautomer, an N-oxide, a hydrate, a solvate, or a salt thereof, or a mixture of same.

### Embodiment C

Of selected interest are compounds of formulas (I), (Ia), (I′) or (I″), wherein R₄ is selected from C(═O)—NRₒRₚ wherein Rₒ and Rₚ are independently selected from
H
C₁₋₄-alkyl or C₃₋₆-cycloalkyl, both optionally substituted one or more times by
—F, —Cl, —Br, —CN;
—OH, —ORᵢ with Rᵢ selected from C₁₋₄-alkyl or C₃₋₆-cycloalkyl, —CF₃, —CHF₂, —CH₂F;
—NRⱼRₖ wherein Rⱼ and Rₖ are independently selected from —H and C₁-C₄-alkyl
phenyl or 5 to 6 membered heteroaryl, both optionally substituted one or more times by
   halogen, —CN,
   —CF₃, —CHF₂, —CH₂F,
   C₁₋₄-alkyl optionally substituted with one or more halogen;
   C₃₋₆-cycloalkyl;
   O—C₁₋₄-alkyl,
   O—C₃₋₆-cycloalky,
   C₁₋₃-alkinyl,
   —NRⱼRₖ wherein Rⱼ and Rₖ are independently selected from —H and C₁-C₄-alkyl
   SR_{d}, SOR_{d}, SO₂R_{d}, SONR_{d} or SON(H)R_{d} wherein R_{d} is selected from methyl, ethyl, cyclopropyl, isopropyl and tert·butyl;

or a stereoisomer, a tautomer, an N-oxide, a hydrate, a solvate, or a salt thereof, or a mixture of same.

### Embodiment C

Of selected interest are compounds of formulas (I), (Ia), (I′) or (I″) (see embodiments 1, A, A′ and A″), wherein A is selected from a single bond or —O— or a stereoisomer, a tautomer, an N-oxide, a hydrate, a solvate, or a salt thereof, or a mixture of same.

### Embodiment D

Of selected interest are compounds of formulas (I), (Ia), (I′) or (I″) (see embodiments 1, A, A′ and A″), wherein E is —CH₂—CH₂— or a stereoisomer, a tautomer, an N-oxide, a hydrate, a solvate, or a salt thereof, or a mixture of same.

Of selected interest are also the following groups of compounds:

### Embodiment E

The compound of formula (I-1)

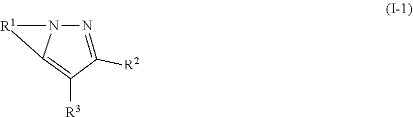

wherein
R¹ is selected from the group consisting of

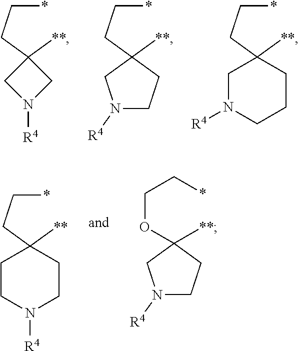

R² is selected from the group consisting of 4-amino-1H-imidazo[4,5-c]pyridin-7-yl, 6-amino-5-cyclohexylmethanesulfonyl-1-oxidopyridin-1-ium-3-yl, and

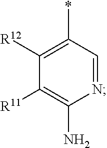

R³ is selected from the group consisting of —H, —Cl, and —Br;
R⁴ is selected from the group consisting of
   C₁₋₄-alkyl or C₃₋₆-cycloalkyl, both optionally substituted one or more times by
      —F, —Cl, —Br, —CN;
      —OH, —ORᵢ wherein Rᵢ is selected from C₁₋₄-alkyl or C₃₋₆Cycloalkyl), —CFₓH_{y} with x and y=0, 1, 2 or 3 and x+y=3;
      —NRⱼRₖ wherein Rⱼ and Rₖ are independently selected from —H and C₁-C₄-alkyl or
      phenyl or 5 to 6 membered heteroaryl, both optionally substituted one or more times by
         halogen, —CN,
         —CFₓH_{y} with x and y=0, 1, 2 or 3 and x+y=3,
         C₁₋₄-alkyl optionally substituted with one or more halogen;
         C₃₋₆-cycloalkyl;
         O—C₁₋₄-alkyl,
         O—C₃₋₆-cycloalky,
         C₁₋₃-alkinyl,
         —NRⱼRₖ wherein Rⱼ and Rₖ are independently selected from —H and C₁-C₄-alkyl
         SR_{d}, SOR_{d}, SO₂R_{d}, SONR_{d} or SON(H)R_{d} wherein R_{d} is selected from methyl, ethyl, cyclopropyl, isopropyl and tert·butyl;
   C(═O)—Rᵢ in which Rᵢ is selected from
      C₁₋₄-alkyl or C₃₋₆-cycloalkyl, both optionally substituted one or more times by
         —F, —Cl, —Br, —CN;
         —OH, —ORᵢ with Rᵢ selected from C₁₋₄-alkyl or C₃₋₆Cycloalkyl), —CFₓH_{y} with x and y=0, 1, 2 or 3 and x+y=3;
         —NRⱼRₖ wherein Rⱼ and Rₖ are independently selected from —H and C₁-C₄-alkyl
         phenyl or 5 to 6 membered heteroaryl, both optionally substituted one or more times by
         halogen, —CN,
         —CFₓH_{y} with x and y=0, 1, 2 or 3 and x+y=3,
         C₁₋₄-alkyl optionally substituted with one or more halogen;
         C₃₋₆-cycloalkyl;
         O—C₁₋₄-alkyl,
         O—C₃₋₆-cycloalky,
         C₁₋₃-alkinyl,
         —NRⱼRₖ wherein Rⱼ and Rₖ are independently selected from —H and C₁-C₄-alkyl
         SR_{d}, SOR_{d}, SO₂R_{d}, SONR_{d} or SON(H)R_{d} wherein R_{d} is selected from methyl, ethyl, cyclopropyl, isopropyl and tert·butyl;
      phenyl or 5 to 6 membered heteroaryl, both optionally substituted one or more times by
         halogen, —CN,
         —CFₓH_{y} with x and y=0, 1, 2 or 3 and x+y=3,
         C₁₋₄-alkyl optionally substituted with one or more halogen;
         C₃₋₆-cycloalkyl;
         O—C₁₋₄-alkyl,
         O—C₃₋₆-cycloalky,
         C₁₋₃-alkinyl,
         —NRⱼRₖ wherein Rⱼ and Rₖ are independently selected from —H and C₁-C₄-alkyl
         SR_{d}, SOR_{d}, SO₂R_{d}, SONR_{d} or SON(H)R_{d} wherein R_{d} is selected from methyl, ethyl, cyclopropyl, isopropyl and tert·butyl;
   C(═O)—NRₒRₚ wherein Rₒ and Rₚ are independently selected from
      H
      C₁₋₄-alkyl or C₃₋₆-cycloalkyl, both optionally substituted one or more times by
         —F, —Cl, —Br, —CN;
         —OH, —ORᵢ with Rᵢ selected from C₁₋₄-alkyl or C₃₋₆Cycloalkyl), —CFₓH_{y} with x and y=0, 1, 2 or 3 and x+y=3;
         —NRⱼRₖ wherein Rⱼ and Rₖ are independently selected from —H and C₁-C₄-alkyl
         phenyl or 5 to 6 membered heteroaryl, both optionally substituted one or more times by
         halogen, —CN,
         —CFₓH_{y} with x and y=0, 1, 2 or 3 and x+y=3,
         C₁₋₄-alkyl optionally substituted with one or more halogen;
         C₃₋₆-cycloalkyl;
         O—C₁₋₄-alkyl,
         O—C₃₋₆-cycloalky,
         C₁₋₃-alkinyl,
         —NRⱼRₖ wherein Rⱼ and Rₖ are independently selected from —H and C₁-C₄-alkyl
         SR_{d}, SOR_{d}, SO₂R_{d}, SONR_{d} or SON(H)R_{d} wherein R_{d} is selected from methyl, ethyl, cyclopropyl, isopropyl and tert·butyl;
      phenyl or 5 to 6 membered heteroaryl, both optionally substituted one or more times by
         halogen, —CN,
         —CFₓH_{y} with x and y=0, 1, 2 or 3 and x+y=3,
         C₁₋₄-alkyl optionally substituted with one or more halogen;
         C₃₋₆-cycloalkyl;
         O—C₁₋₄-alkyl,
         O—C₃₋₆-cycloalky,
         C₁₋₃-alkinyl,
         —NRⱼRₖ wherein Rⱼ and Rₖ are independently selected from —H and C₁-C₄-alkyl
         SR_{d}, SOR_{d}, SO₂R_{d}, SONR_{d} or SON(H)R_{d} wherein R_{d} is selected from methyl, ethyl, cyclopropyl, isopropyl and tert·butyl;
R¹¹ is selected from the group consisting of —H, —CH₃, —F, —Cl, —Br, —CN, —OCH₃, —CH₂OH, —SCH₃, cyclopropyl, —NO₂, —NHCCH₃O, —P(CH₃)₂O, —C(CH₃)₂R³⁷, —COOR¹⁸, —CF₂R³⁸, —SO₂R³⁹, —SC(CH₃)₃, 1,2-thiazol-5-yl, —SCF₃, —OCF₂R²⁷, pyridin-4-yl, 1-methyl-1H-pyrazol-3-yl, —CON(R¹⁶)CH₂R¹⁸, —COC(R²⁷)₃, 1-methylpiperidin-4-yl, 1-methyl-1,2,3,6-tetrahydropyridin-4-yl, 1,5-dimethyl-1H-pyrazol-4-yl, (cyclopentylmethyl)sulfanyl, 1-(pyridin-4-yl)ethoxy, 5H,6H,7H-cyclopenta[b]pyridin-7-yloxy,

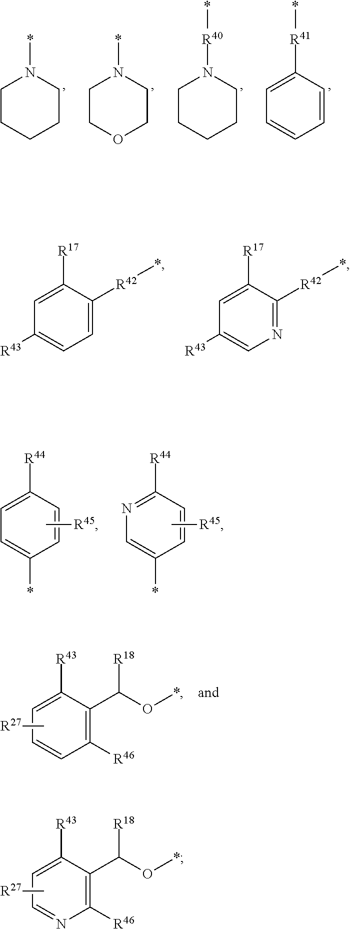

R¹² is selected from the group consisting of —H, —CH₃, —F, and —CF₃;
R¹⁶ is selected from the group consisting of —H, —CH₃, and —CH₂CH₃;
R¹⁸ is selected from the group consisting of —H and —CH₃;
R²⁷ is selected from the group consisting of —H and —F;
R³⁷ is selected from the group consisting of —H and —OH;
R³⁸ is selected from the group consisting of —H, —CH₃, and —F;
R³⁹ is selected from the group consisting of —CH₃, —NH₂, —NHCH₃, —N(CH₃)₂, morpholin-4-yl, and 4-methylpiperazin-1-yl;
R⁴⁰ is selected from the group consisting of —NHCO—, —CHCH₃S—, —CH₂SO—, —CH₂SO₂—, and —N—CH₂—, —O—CH₂—, and —S—CH₂—;
R⁴¹ is selected from the group consisting of —CH₂S—, —SO—, —NHCO—, —CHCH₃S—, —O—, and —S—;
R⁴² is selected from the group consisting of —CH₂O—, —CHCH₃O—, and —CHCH₃NHCO—;
R⁴³ is selected from the group consisting of —H and —Cl;
R⁴⁴ is selected from the group consisting of —H, —CH₃, —CCH₃O, —SCH₃O, —SO₂R⁶⁴, and dimethoxyphosphoryl;
R⁴⁵ is selected from the group consisting of —H, —CH₃, —F, —OCH₃, and —SCH₃O₂;
R⁴⁶ is selected from the group consisting of —H, —F, and —Cl;
R⁶⁴ is selected from the group consisting of —CH₃, —NH₂, —NHCH₃, cyclopropyl, and —N(CH₃)₂;
or a stereoisomer, a tautomer, an N-oxide, a hydrate, a solvate, or a salt thereof, or a mixture of same.

### Embodiment F

Compound according to embodiment E wherein
R¹ is selected from the group consisting of

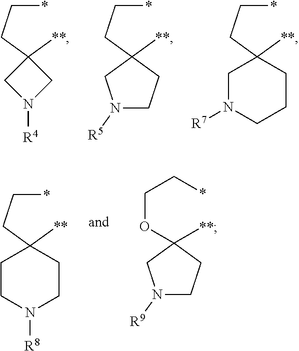

R² is selected from the group consisting of 4-amino-1H-imidazo[4,5-c]pyridin-7-yl, 6-amino-5-cyclohexylmethanesulfonyl-1-oxidopyridin-1-ium-3-yl, and

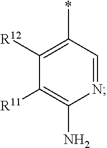

R³ is selected from the group consisting of —H, —Cl, and —Br;
R⁴ is selected from the group consisting of —CONHR¹³, (1H-imidazol-2-yl)methyl, and —COOC(CH₃)₃;
R⁵ is selected from the group consisting of —COOC(R¹⁷)(R¹⁸)₂, —C(═R²¹)NHC(R¹⁸)(R¹⁹)R²⁰,

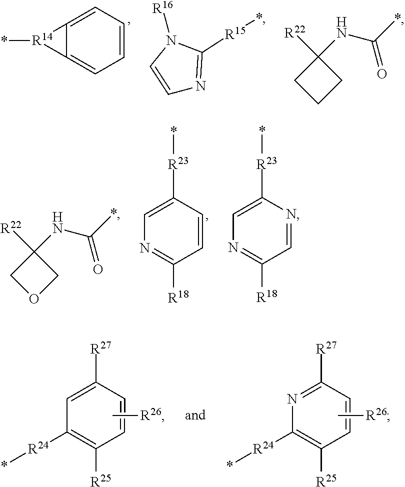

R⁷ is selected from the group consisting of —CONHR²⁸ and —COOC(CH₃)₃;
R⁸ is selected from the group consisting of —CONHR²⁹, (1H-imidazol-2-yl)methyl, and —COOC(CH₃)₃;
R⁹ is selected from the group consisting of —CONHR³⁰, (1H-imidazol-2-yl)methyl, and —COOC(CH₃)₃;
R¹¹ is selected from the group consisting of —H, —CH₃, —F, —Cl, —Br, —CN, —OCH₃, —CH₂OH, —SCH₃, cyclopropyl, —NO₂, —NHCCH₃O, —P(CH₃)₂O, —C(CH₃)₂R³⁷, —COOR¹⁸, —CF₂R³⁸, —SO₂R³⁹, —SC(CH₃)₃, 1,2-thiazol-5-yl, —SCF₃, —OCF₂R²⁷, pyridin-4-yl, 1-methyl-1H-pyrazol-3-yl, —CON(R¹⁶)CH₂R¹⁸, —COC(R²⁷)₃, 1-methylpiperidin-4-yl, 1-methyl-1,2,3,6-tetrahydropyridin-4-yl, 1,5-dimethyl-1H-pyrazol-4-yl, (cyclopentylmethyl)sulfanyl, 1-(pyridin-4-yl)ethoxy, 5H,6H,7H-cyclopenta[b]pyridin-7-yloxy,

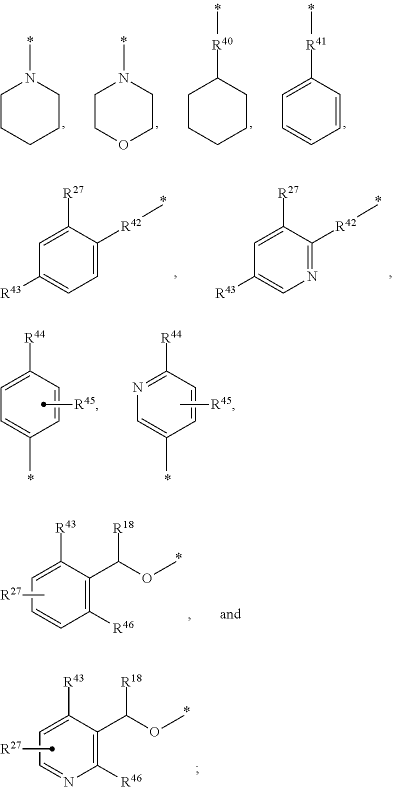

R¹² is selected from the group consisting of —H, —CH₃, —F, and —CF₃;
R¹³ is selected from the group consisting of —CH₂CH₃, —C(CH₃)₂R⁴⁷, bicyclo[2.2.1]heptan-1-yl, (pyrimidin-4-yl)methyl, 1-(1,2-oxazol-3-yl)ethyl, 1-(5-fluoropyridin-3-yl)ethyl, 1-(1,3,4-trimethyl-1H-pyrazol-5-yl)ethyl,

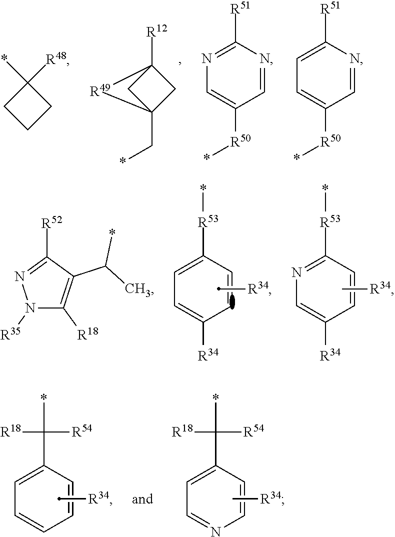

R¹⁴ is selected from the group consisting of

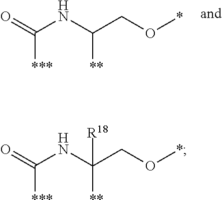

R¹⁵ is selected from the group consisting of —CH₂— and —CHCH₃NHCO—;
R¹⁶ is selected from the group consisting of —H, —CH₃, and —CH₂CH₃;
R¹⁷ is selected from the group consisting of —CH₃ and phenyl;
R¹⁸ is selected from the group consisting of —H and —CH₃;
R¹⁹ is selected from the group consisting of —CH₃, cyclopropyl, 1,2-oxazol-3-yl, pyridazin-3-yl, pyridazin-4-yl, oxan-4-yl, 1-methyl-1H-1,2,3-triazol-4-yl, 1-methylpiperidin-4-yl, 1,3,4-trimethyl-1H-pyrazol-5-yl, 1-methyl-2-oxopiperidin-4-yl, 1-methyl-1H-1,3-benzodiazol-2-yl,

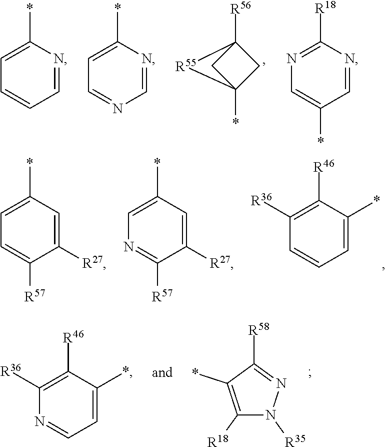

R²⁰ is selected from the group consisting of —H, —CH₃, —CH₂CH₃, —CH(CH₃)₂, and —CH₂OCH₃;
R²¹ is selected from the group consisting of ═O and ═NCN;
R²² is selected from the group consisting of —H, pyridin-4-yl, pyridin-3-yl,

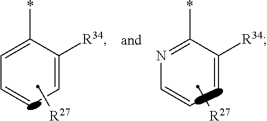

R²³ is selected from the group consisting of —C(CH₃)₂CO— and —CH(R¹⁸)NHCO—;
R²⁴ is selected from the group consisting of —NHCO—, —C(CH₃)₂CO—, —C(R⁵⁹)(R⁶⁰)NHCO—,

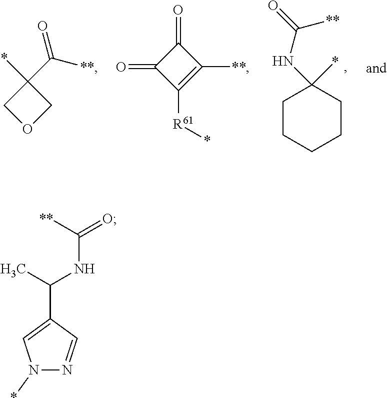

R²⁵ is selected from the group consisting of —H, —F, —Cl, —CN, and —SO₂NHCH₃;
R²⁶ is selected from the group consisting of —H, —F, —CN, and —CF₃;
R²⁷ is selected from the group consisting of —H and —F;
R²⁸ is selected from the group consisting of —CH₂CH₃, —CH(CH₃)₂, and cyclobutyl;
R²⁹ is selected from the group consisting of —CH₂CH₃, —CH(CH₃)₂, cyclobutyl, and

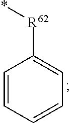

R³⁰ is selected from the group consisting of —CH₂CH₃, cyclobutyl, —C(CH₃)₂R⁶³, 1-(5-fluoropyridin-3-yl)ethyl,

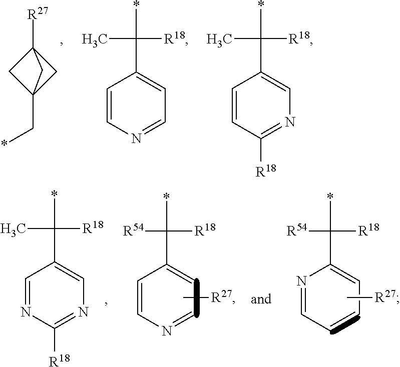

R³⁴ is selected from the group consisting of —H, —F, —Cl, and —CN;
R³⁵ is selected from the group consisting of —CH₃ and —CH₂CH₃;
R³⁶ is selected from the group consisting of —H, —F, and —CN;
R³⁷ is selected from the group consisting of —H and —OH;
R³⁸ is selected from the group consisting of —H, —CH₃, and —F;
R³⁹ is selected from the group consisting of —CH₃, —NH₂, —NHCH₃, —N(CH₃)₂, morpholin-4-yl, and 4-methylpiperazin-1-yl;
R⁴⁰ is selected from the group consisting of —NHCO—, —CHCH₃S—, —CH₂SO—, —CH₂SO₂—, and —N—CH₂—, —O—CH₂—, and —S—CH₂—;
R⁴¹ is selected from the group consisting of —CH₂S—, —SO—, —NHCO—, —CHCH₃S—, —O—, and —S—;
R⁴² is selected from the group consisting of —CH₂O—, —CHCH₃O—, and —CHCH₃NHCO—;
R⁴³ is selected from the group consisting of —H and —Cl;
R⁴⁴ is selected from the group consisting of —H, —CH₃, —CCH₃O, —SCH₃O, —SO₂R⁶⁴, and dimethoxyphosphoryl;
R⁴⁵ is selected from the group consisting of —H, —CH₃, —F, —OCH₃, and —SCH₃O₂;
R⁴⁶ is selected from the group consisting of —H, —F, and —Cl;
R⁴⁷ is selected from the group consisting of —H, 1-methyl-1H-1,2,3-triazol-4-yl,

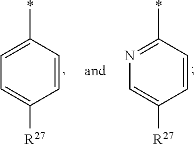

R⁴⁸ is selected from the group consisting of —H, —CH₃, —CH(CH₃)₂, and pyridin-4-yl;
R⁴⁹ is selected from the group consisting of —CH₂— and —CH₂O—;
R⁵⁰ is selected from the group consisting of —CH₂—, —CHCH₃—, —C(CH₃)₂—, and

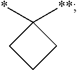

R⁵¹ is selected from the group consisting of —H, —CH₃, and —CN;
R⁵² is selected from the group consisting of —H, —CH₃, and —CHF₂;
R⁵³ is selected from the group consisting of —CH₂—, —CHCH₃—, —C(CH₃)₂—,

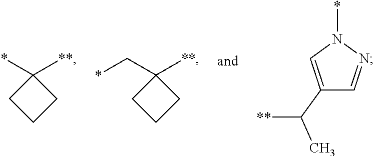

R⁵⁴ is selected from the group consisting of —H, —CH₃, —CH₂CH₃, and —CH₂OCH₃;
R⁵⁵ is selected from the group consisting of —CH₂— and —OCH₂—;
R⁵⁶ is selected from the group consisting of —CH₃ and —CF₃;
R⁵⁷ is selected from the group consisting of —H, —CH₃, —F, and —CN;
R⁵⁸ is selected from the group consisting of —H, —CH₃, and —CF₃;
R⁵⁹ is selected from the group consisting of —H, —CH₃, cyclopropyl, —CH₂OCH₃, and —CF₃;
R⁶⁰ is selected from the group consisting of —H, —CH₃, and —OCH₃;
R⁶¹ is selected from the group consisting of —NH— and —NHCH₂—;
R⁶² is selected from the group consisting of —CH₂— and

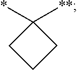

R⁶³ is selected from the group consisting of —H, 1-methyl-1H-1,2,3-triazol-4-yl, and 3-chloropyridin-4-yl;
R⁶⁴ is selected from the group consisting of —CH₃, —NH₂, —NHCH₃, cyclopropyl, and —N(CH₃)₂;
or a stereoisomer, a tautomer, an N-oxide, a hydrate, a solvate, or a salt thereof, or a mixture of same.

### Embodiment G

The compound according to embodiment E wherein
R¹ is selected from

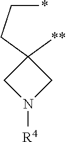

or a stereoisomer, a tautomer, an N-oxide, a hydrate, a solvate, or a salt thereof, or a mixture of same.

### Embodiment H

The compound according to embodiment E wherein
R¹ is selected from

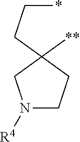

or a stereoisomer, a tautomer, an N-oxide, a hydrate, a solvate, or a salt thereof, or a mixture of same.

### Embodiment I

The compound according to embodiment E wherein
R¹ is selected from the group consisting of

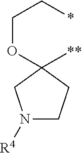

or a stereoisomer, a tautomer, an N-oxide, a hydrate, a solvate, or a salt thereof, or a mixture of same.

### Embodiment J

The compound according to embodiment F wherein
R¹ is selected from

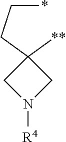

or a stereoisomer, a tautomer, an N-oxide, a hydrate, a solvate, or a salt thereof, or a mixture of same.

### Embodiment K

The compound according to embodiment F wherein
R¹ is selected from

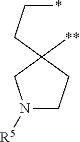

or a stereoisomer, a tautomer, an N-oxide, a hydrate, a solvate, or a salt thereof, or a mixture of same.

### Embodiment L

The compound according to embodiment F wherein
R¹ is selected from

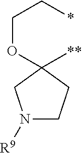

or a stereoisomer, a tautomer, an N-oxide, a hydrate, a solvate, or a salt thereof, or a mixture of same.

and their polymorphs, enantiomers, diastereomers, racemates, tautomers, solvates, physiologically acceptable salts and solvates of these salts.

### Embodiment M

### Compound of Formula (I-2)

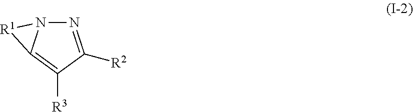 [1618] wherein
R¹ is selected from the group consisting of

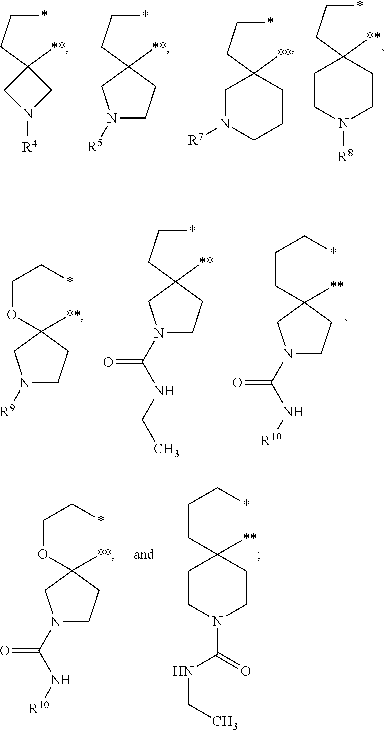

R² is selected from the group consisting of 4-amino-1H-imidazo[4,5-c]pyridin-7-yl, 6-amino-5-cyclohexylmethanesulfonyl-1-oxidopyridin-1-ium-3-yl, and

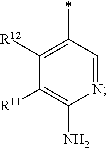

R³ is selected from the group consisting of —H, —Cl, and —Br;
R⁴ is selected from the group consisting of —CONHR¹³, (1H-imidazol-2-yl)methyl, and —COOC(CH₃)₃;
R⁵ is selected from the group consisting of —COOC(R¹⁷)(R¹⁸)₂, —C(═R²¹)NHC(R¹⁸)(R¹⁹)R²⁰,

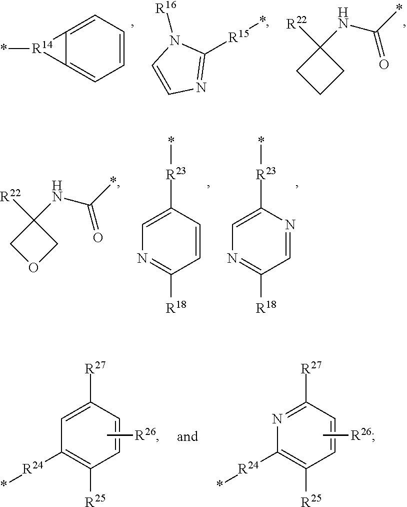

R⁷ is selected from the group consisting of —CONHR²⁸ and —COOC(CH₃)₃;
R⁸ is selected from the group consisting of —CONHR²⁹, (1H-imidazol-2-yl)methyl, and —COOC(CH₃)₃;
R⁹ is selected from the group consisting of —CONHR³⁰, (1H-imidazol-2-yl)methyl, and —COOC(CH₃)₃;
R¹⁰ is selected from the group consisting of —CH₂CH₃, {1-methyl-2-oxabicyclo[2.1.1]hexan-4-yl}methyl, 1-(1,3,4-trimethyl-1H-pyrazol-5-yl)ethyl,

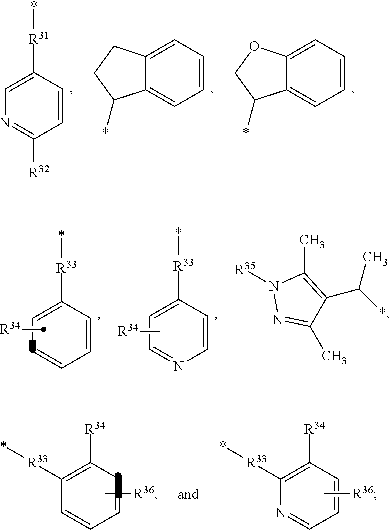

R¹¹ is selected from the group consisting of —H, —CH₃, —F, —Cl, —Br, —CN, —OCH₃, —CH₂OH, —SCH₃, cyclopropyl, —NO₂, —NHCCH₃O, —P(CH₃)₂O, —C(CH₃)₂R³⁷, —COOR¹⁸, —CF₂R³⁸, —SO₂R³⁹, —SC(CH₃)₃, 1,2-thiazol-5-yl, —SCF₃, —OCF₂R²⁷, pyridin-4-yl, 1-methyl-1H-pyrazol-3-yl, —CON(R¹⁶)CH₂R¹⁸, —COC(R²⁷)₃, 1-methylpiperidin-4-yl, 1-methyl-1,2,3,6-tetrahydropyridin-4-yl, 1,5-dimethyl-1H-pyrazol-4-yl, (cyclopentylmethyl)sulfanyl, 1-(pyridin-4-yl)ethoxy, 5H,6H,7H-cyclopenta[b]pyridin-7-yloxy,

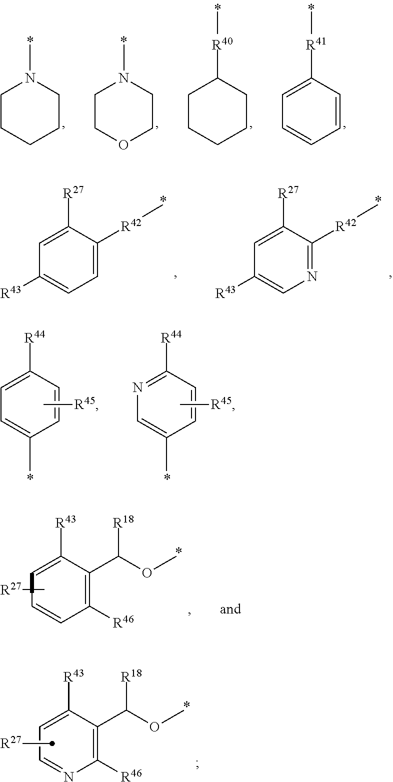

R¹² is selected from the group consisting of —H, —CH₃, —F, and —CF₃;
R¹³ is selected from the group consisting of —CH₂CH₃, —C(CH₃)₂R⁴⁷, bicyclo[2.2.1]heptan-1-yl, (pyrimidin-4-yl)methyl, 1-(1,2-oxazol-3-yl)ethyl, 1-(5-fluoropyridin-3-yl)ethyl, 1-(1,3,4-trimethyl-1H-pyrazol-5-yl)ethyl,

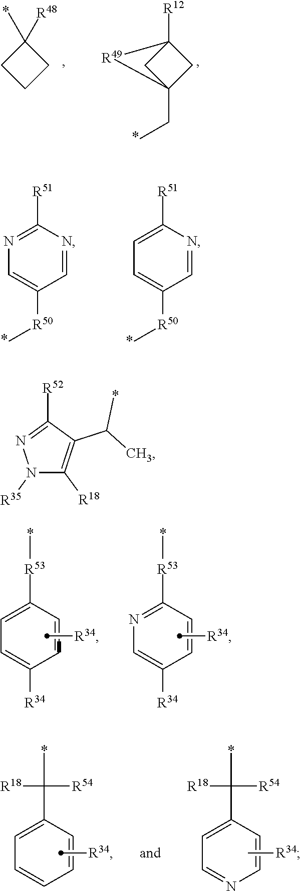

R¹⁴ is selected from the group consisting of

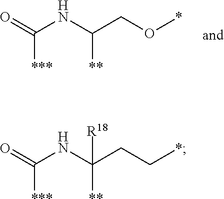

R¹⁵ is selected from the group consisting of —CH₂— and —CHCH₃NHCO—;
R¹⁶ is selected from the group consisting of —H, —CH₃, and —CH₂CH₃;
R¹⁷ is selected from the group consisting of —CH₃ and phenyl;
R¹⁸ is selected from the group consisting of —H and —CH₃;
R¹⁹ is selected from the group consisting of —CH₃, cyclopropyl, 1,2-oxazol-3-yl, pyridazin-3-yl, pyridazin-4-yl, oxan-4-yl, 1-methyl-1H-1,2,3-triazol-4-yl, 1-methylpiperidin-4-yl, 1,3,4-trimethyl-1H-pyrazol-5-yl, 1-methyl-2-oxopiperidin-4-yl, 1-methyl-1H-1,3-benzodiazol-2-yl,

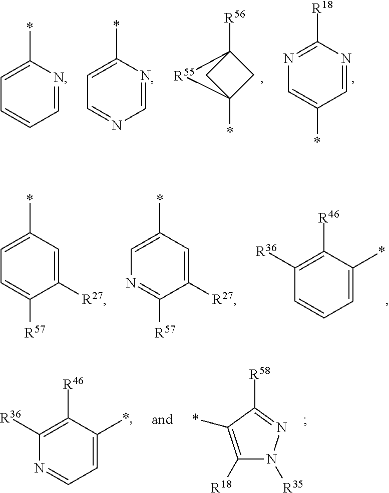

R²⁰ is selected from the group consisting of —H, —CH₃, —CH₂CH₃, —CH(CH₃)₂, and —CH₂OCH₃;
R²¹ is selected from the group consisting of ═O and ═NCN;
R²² is selected from the group consisting of —H, pyridin-4-yl, pyridin-3-yl,

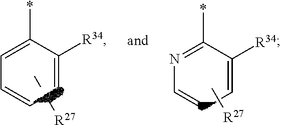

R²³ is selected from the group consisting of —C(CH₃)₂CO— and —CH(R¹⁸)NHCO—;
R²⁴ is selected from the group consisting of —NHCO—, —C(CH₃)₂CO—, —C(R⁵⁹)(R⁶⁰)NHCO—,

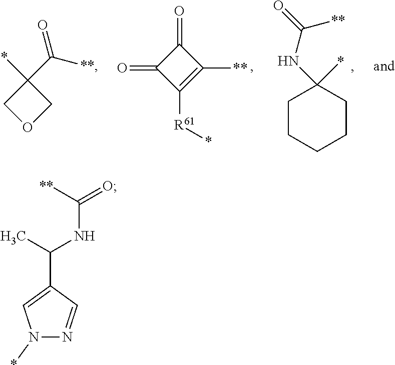

R²⁵ is selected from the group consisting of —H, —F, —Cl, —CN, and —SO₂NHCH₃;
R²⁶ is selected from the group consisting of —H, —F, —CN, and —CF₃;
R²⁷ is selected from the group consisting of —H and —F;
R²⁸ is selected from the group consisting of —CH₂CH₃, —CH(CH₃)₂, and cyclobutyl;
R²⁹ is selected from the group consisting of —CH₂CH₃, —CH(CH₃)₂, cyclobutyl, and

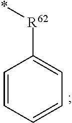

R³⁰ is selected from the group consisting of —CH₂CH₃, cyclobutyl, —C(CH₃)₂R⁶³, 1-(5-fluoropyridin-3-yl)ethyl,

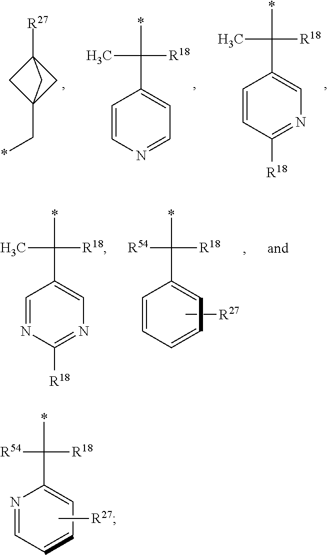

R³¹ is selected from the group consisting of —CHCH₃— and

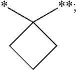

R³² is selected from the group consisting of —H and —CN;
R³³ is selected from the group consisting of —CHCH₃—, —C(CH₃)₂—, and

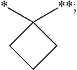

R³⁴ is selected from the group consisting of —H, —F, —Cl, and —CN;
R³⁵ is selected from the group consisting of —CH₃ and —CH₂CH₃;
R³⁶ is selected from the group consisting of —H, —F, and —CN;
R³⁷ is selected from the group consisting of —H and —OH;
R³⁸ is selected from the group consisting of —H, —CH₃, and —F;
R³⁹ is selected from the group consisting of —CH₃, —NH₂, —NHCH₃, —N(CH₃)₂, morpholin-4-yl, and 4-methylpiperazin-1-yl;
R⁴⁰ is selected from the group consisting of —NHCO—, —CHCH₃S—, —CH₂SO—, —CH₂SO₂—, and **—N—CH₂—*, **—O—CH₂—*, and **—S—CH₂—*;
R⁴¹ is selected from the group consisting of —CH₂S—, —SO—, —NHCO—, —CHCH₃S—, —O—, and —S—;
R⁴² is selected from the group consisting of —CH₂O—, —CHCH₃O—, and —CHCH₃NHCO—;
R⁴³ is selected from the group consisting of —H and —Cl;
R⁴⁴ is selected from the group consisting of —H, —CH₃, —CCH₃O, —SCH₃O, —SO₂R⁶⁴, and dimethoxyphosphoryl;
R⁴⁵ is selected from the group consisting of —H, —CH₃, —F, —OCH₃, and —SCH₃O₂;
R⁴⁶ is selected from the group consisting of —H, —F, and —Cl;
R⁴⁷ is selected from the group consisting of —H, 1-methyl-1H-1,2,3-triazol-4-yl,

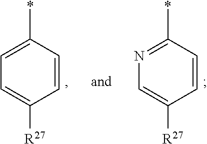

R⁴⁸ is selected from the group consisting of —H, —CH₃, —CH(CH₃)₂, and pyridin-4-yl;
R⁴⁹ is selected from the group consisting of —CH₂— and —CH₂O—;
R⁵⁰ is selected from the group consisting of —CH₂—, —CHCH₃—, —C(CH₃)₂—, and

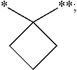

R⁵¹ is selected from the group consisting of —H, —CH₃, and —CN;
R⁵² is selected from the group consisting of —H, —CH₃, and —CHF₂;
R⁵³ is selected from the group consisting of —CH₂—, —CHCH₃—, —C(CH₃)₂—,

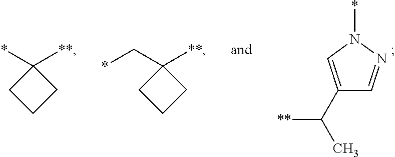

R⁵⁴ is selected from the group consisting of —H, —CH₃, —CH₂CH₃, and —CH₂OCH₃;
R⁵⁵ is selected from the group consisting of —CH₂— and —OCH₂—;
R⁵⁶ is selected from the group consisting of —CH₃ and —CF₃;
R⁵⁷ is selected from the group consisting of —H, —CH₃, —F, and —CN;
R⁵⁸ is selected from the group consisting of —H, —CH₃, and —CF₃;
R⁵⁹ is selected from the group consisting of —H, —CH₃, cyclopropyl, —CH₂OCH₃, and —CF₃;
R⁶⁰ is selected from the group consisting of —H, —CH₃, and —OCH₃;
R⁶¹ is selected from the group consisting of —NH— and —NHCH₂—;
R⁶² is selected from the group consisting of —CH₂— and

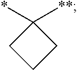

R⁶³ is selected from the group consisting of —H, 1-methyl-1H-1,2,3-triazol-4-yl, and 3-chloropyridin-4-yl;
R⁶⁴ is selected from the group consisting of —CH₃, —NH₂, —NHCH₃, cyclopropyl, and —N(CH₃)₂; or a stereoisomer, a tautomer, an N-oxide, a hydrate, a solvate, or a salt thereof, or a mixture of same.

### Embodiment N

A compound according to embodiment M of formula (I-3)

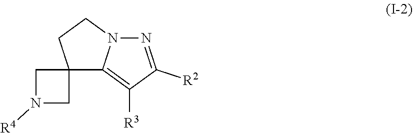

wherein
R² is selected from the group consisting of 4-amino-1H-imidazo[4,5-c]pyridin-7-yl, 6-amino-5-cyclohexylmethanesulfonyl-1-oxidopyridin-1-ium-3-yl, and

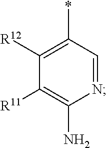

R³ is selected from the group consisting of —H, —Cl, and —Br;
R⁴ is selected from the group consisting of —CONHR¹³, (1H-imidazol-2-yl)methyl, and —COOC(CH₃)₃;
R¹¹ is selected from the group consisting of —H, —CH₃, —F, —Cl, —Br, —CN, —OCH₃, —CH₂OH, —SCH₃, cyclopropyl, —NO₂, —NHCCH₃O, —P(CH₃)₂O, —C(CH₃)₂R³⁷, —COOR¹⁸, —CF₂R³⁸, —SO₂R³⁹, —SC(CH₃)₃, 1,2-thiazol-5-yl, —SCF₃, —OCF₂R²⁷, pyridin-4-yl, 1-methyl-1H-pyrazol-3-yl, —CON(R¹⁶)CH₂R¹⁸, —COC(R²⁷)₃, 1-methylpiperidin-4-yl, 1-methyl-1,2,3,6-tetrahydropyridin-4-yl, 1,5-dimethyl-1H-pyrazol-4-yl, (cyclopentylmethyl)sulfanyl, 1-(pyridin-4-yl)ethoxy, 5H,6H,7H-cyclopenta[b]pyridin-7-yloxy,

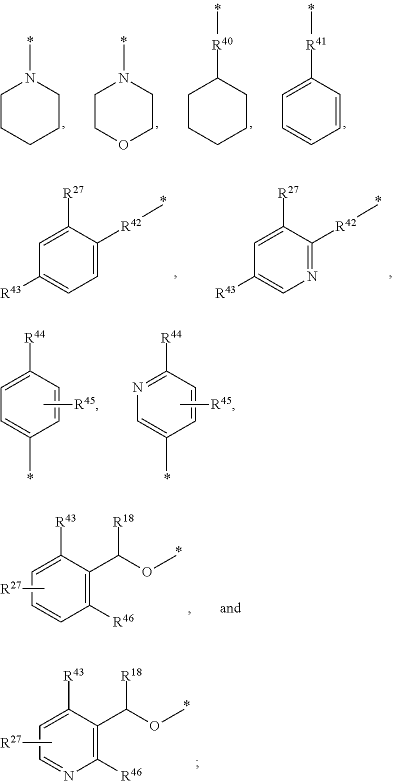

R¹² is selected from the group consisting of —H, —CH₃, —F, and —CF₃;
R¹³ is selected from the group consisting of —CH₂CH₃, —C(CH₃)₂R⁴⁷, bicyclo[2.2.1]heptan-1-yl, (pyrimidin-4-yl)methyl, 1-(1,2-oxazol-3-yl)ethyl, 1-(5-fluoropyridin-3-yl)ethyl, 1-(1,3,4-trimethyl-1H-pyrazol-5-yl)ethyl,

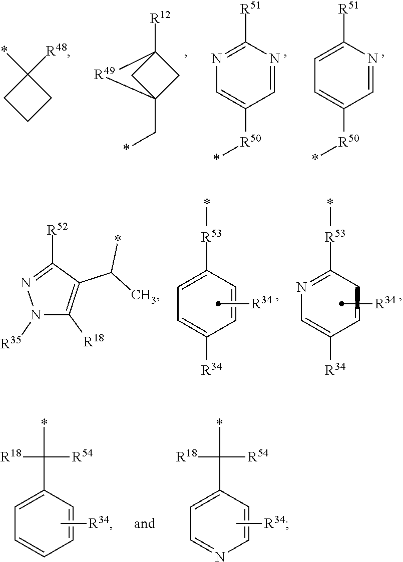

R¹⁶ is selected from the group consisting of —H, —CH₃, and —CH₂CH₃;
R¹⁸ is selected from the group consisting of —H and —CH₃;
R²⁷ is selected from the group consisting of —H and —F;
R³⁴ is selected from the group consisting of —H, —F, —Cl, and —CN;
R³⁵ is selected from the group consisting of —CH₃ and —CH₂CH₃;
R³⁷ is selected from the group consisting of —H and —OH;
R³⁸ is selected from the group consisting of —H, —CH₃, and —F;
R³⁹ is selected from the group consisting of —CH₃, —NH₂, —NHCH₃, —N(CH₃)₂, morpholin-4-yl, and 4-methylpiperazin-1-yl;
R⁴⁰ is selected from the group consisting of —NHCO—, —CHCH₃S—, —CH₂SO—, —CH₂SO₂—, and **—N—CH₂—*, **—O—CH₂—*, and **—S—CH₂—*;
R⁴¹ is selected from the group consisting of —CH₂S—, —SO—, —NHCO—, —CHCH₃S—, —O—, and —S—;
R⁴² is selected from the group consisting of —CH₂O—, —CHCH₃O—, and —CHCH₃NHCO—;
R⁴³ is selected from the group consisting of —H and —Cl;
R⁴⁴ is selected from the group consisting of —H, —CH₃, —CCH₃O, —SCH₃O, —SO₂R⁶⁴, and dimethoxyphosphoryl;
R⁴⁵ is selected from the group consisting of —H, —CH₃, —F, —OCH₃, and —SCH₃O₂;
R⁴⁶ is selected from the group consisting of —H, —F, and —Cl;
R⁴⁷ is selected from the group consisting of —H, 1-methyl-1H-1,2,3-triazol-4-yl,

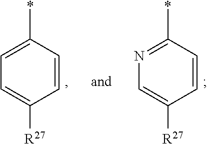

R⁴⁸ is selected from the group consisting of —H, —CH₃, —CH(CH₃)₂, and pyridin-4-yl;
R⁴⁹ is selected from the group consisting of —CH₂— and —CH₂O—;
R⁵⁰ is selected from the group consisting of —CH₂—, —CHCH₃—, —C(CH₃)₂—, and

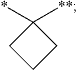

R⁵¹ is selected from the group consisting of —H, —CH₃, and —CN;
R⁵² is selected from the group consisting of —H, —CH₃, and —CHF₂;
R⁵³ is selected from the group consisting of —CH₂—, —CHCH₃—, —C(CH₃)₂—,

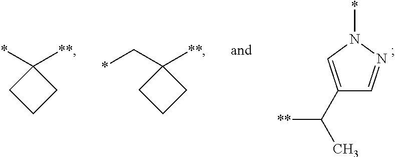

R⁵⁴ is selected from the group consisting of —H, —CH₃, —CH₂CH₃, and —CH₂OCH₃;
R⁶⁴ is selected from the group consisting of —CH₃, —NH₂, —NHCH₃, cyclopropyl, and —N(CH₃)₂;
or a stereoisomer, a tautomer, an N-oxide, a hydrate, a solvate, or a salt thereof, or a mixture of same.

### Embodiment O

Compound according to embodiment M of formula (I-4)

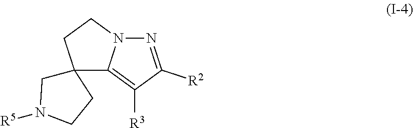

wherein
R² is selected from the group consisting of 4-amino-1H-imidazo[4,5-c]pyridin-7-yl, 6-amino-5-cyclohexylmethanesulfonyl-1-oxidopyridin-1-ium-3-yl, and

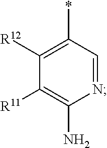

R³ is selected from the group consisting of —H, —Cl, and —Br;
R⁵ is selected from the group consisting of —COOC(R¹⁷)(R¹⁸)₂, —C(═R²¹)NHC(R¹⁸)(R¹⁹)R²⁰,

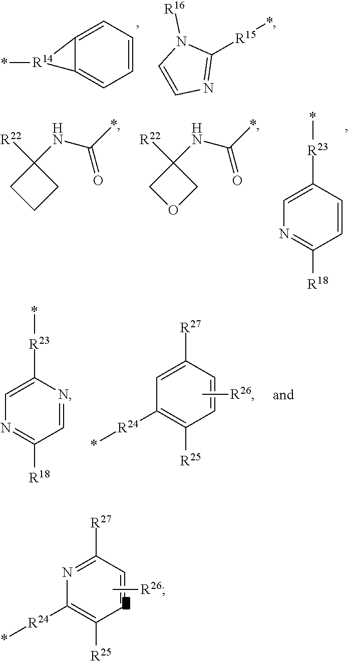

R¹¹ is selected from the group consisting of —H, —CH₃, —F, —Cl, —Br, —CN, —OCH₃, —CH₂OH, —SCH₃, cyclopropyl, —NO₂, —NHCCH₃O, —P(CH₃)₂O, —C(CH₃)₂R³⁷, —COOR¹⁸, —CF₂R³⁸, —SO₂R³⁹, —SC(CH₃)₃, 1,2-thiazol-5-yl, —SCF₃, —OCF₂R²⁷, pyridin-4-yl, 1-methyl-1H-pyrazol-3-yl, —CON(R¹⁶)CH₂R¹⁸, —COC(R²⁷)₃, 1-methylpiperidin-4-yl, 1-methyl-1,2,3,6-tetrahydropyridin-4-yl, 1,5-dimethyl-1H-pyrazol-4-yl, (cyclopentylmethyl)sulfanyl, 1-(pyridin-4-yl)ethoxy, 5H,6H,7H-cyclopenta[b]pyridin-7-yloxy,

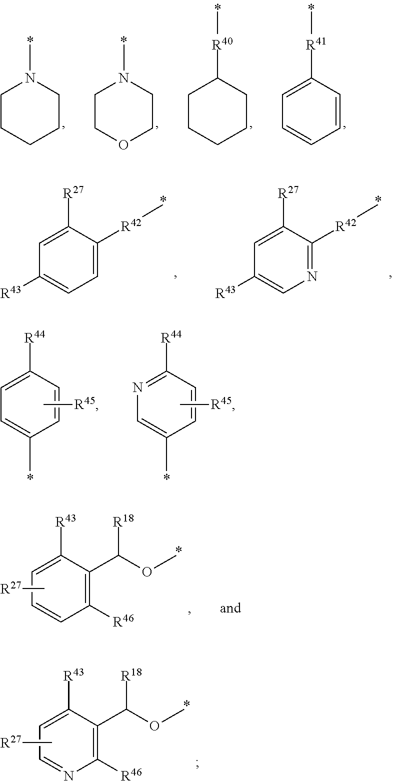

R¹² is selected from the group consisting of —H, —CH₃, —F, and —CF₃;
R¹⁴ is selected from the group consisting of

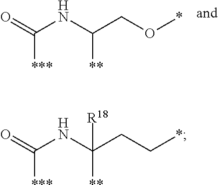

R¹⁵ is selected from the group consisting of —CH₂— and —CHCH₃NHCO—;
R¹⁶ is selected from the group consisting of —H, —CH₃, and —CH₂CH₃;
R¹⁷ is selected from the group consisting of —CH₃ and phenyl;
R¹⁸ is selected from the group consisting of —H and —CH₃;
R¹⁹ is selected from the group consisting of —CH₃, cyclopropyl, 1,2-oxazol-3-yl, pyridazin-3-yl, pyridazin-4-yl, oxan-4-yl, 1-methyl-1H-1,2,3-triazol-4-yl, 1-methylpiperidin-4-yl, 1,3,4-trimethyl-1H-pyrazol-5-yl, 1-methyl-2-oxopiperidin-4-yl, 1-methyl-1H-1,3-benzodiazol-2-yl,

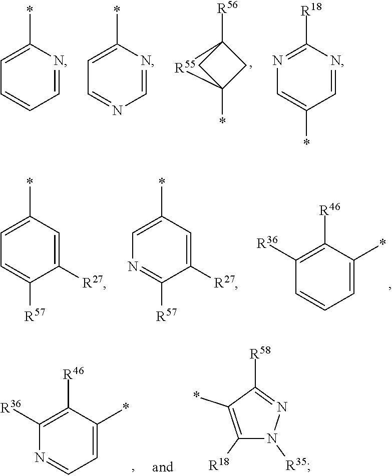

R²⁰ is selected from the group consisting of —H, —CH₃, —CH₂CH₃, —CH(CH₃)₂, and —CH₂OCH₃;
R²¹ is selected from the group consisting of ═O and ═NCN;
R²² is selected from the group consisting of —H, pyridin-4-yl, pyridin-3-yl,

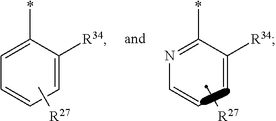

R²³ is selected from the group consisting of —C(CH₃)₂CO— and —CH(R¹⁸)NHCO—;
R²⁴ is selected from the group consisting of —NHCO—, —C(CH₃)₂CO—, —C(R⁵⁹)(R⁶⁰)NHCO—,

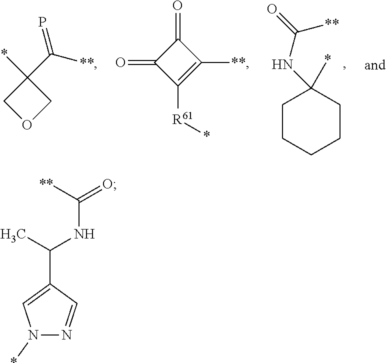

R²⁵ is selected from the group consisting of —H, —F, —Cl, —CN, and —SO₂NHCH₃;
R²⁶ is selected from the group consisting of —H, —F, —CN, and —CF₃;
R²⁷ is selected from the group consisting of —H and —F;
R³⁴ is selected from the group consisting of —H, —F, —Cl, and —CN;
R³⁵ is selected from the group consisting of —CH₃ and —CH₂CH₃;
R³⁶ is selected from the group consisting of —H, —F, and —CN;
R³⁷ is selected from the group consisting of —H and —OH;
R³⁸ is selected from the group consisting of —H, —CH₃, and —F;
R³⁹ is selected from the group consisting of —CH₃, —NH₂, —NHCH₃, —N(CH₃)₂, morpholin-4-yl, and 4-methylpiperazin-1-yl;
R⁴⁰ is selected from the group consisting of —NHCO—, —CHCH₃S—, —CH₂SO—, —CH₂SO₂—, and **—N—CH₂—*, **—O—CH₂—*, and **—S—CH₂—*;
R⁴¹ is selected from the group consisting of —CH₂S—, —SO—, —NHCO—, —CHCH₃S—, —O—, and —S—;
R⁴² is selected from the group consisting of —CH₂O—, —CHCH₃O—, and —CHCH₃NHCO—;
R⁴³ is selected from the group consisting of —H and —Cl;
R⁴⁴ is selected from the group consisting of —H, —CH₃, —CCH₃O, —SCH₃O, —SO₂R⁶⁴, and dimethoxyphosphoryl;
R⁴⁵ is selected from the group consisting of —H, —CH₃, —F, —OCH₃, and —SCH₃O₂;
R⁴⁶ is selected from the group consisting of —H, —F, and —Cl;
R⁵⁵ is selected from the group consisting of —CH₂— and —OCH₂—;
R⁵⁶ is selected from the group consisting of —CH₃ and —CF₃;
R¹⁷ is selected from the group consisting of —H, —CH₃, —F, and —CN;
R⁵⁸ is selected from the group consisting of —H, —CH₃, and —CF₃;
R⁵⁹ is selected from the group consisting of —H, —CH₃, cyclopropyl, —CH₂OCH₃, and —CF₃;
R⁶⁰ is selected from the group consisting of —H, —CH₃, and —OCH₃;
R⁶¹ is selected from the group consisting of —NH— and —NHCH₂—;
R⁶⁴ is selected from the group consisting of —CH₃, —NH₂, —NHCH₃, cyclopropyl, and —N(CH₃)₂;
or a stereoisomer, a tautomer, an N-oxide, a hydrate, a solvate, or a salt thereof, or a mixture of same.

### Embodiment P

A compound according to embodiment M of formula (I-5)

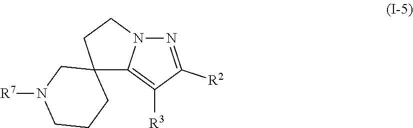

wherein
R² is selected from the group consisting of 4-amino-1H-imidazo[4,5-c]pyridin-7-yl, 6-amino-5-cyclohexylmethanesulfonyl-1-oxidopyridin-1-ium-3-yl, and

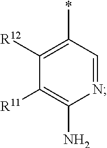

R³ is selected from the group consisting of —H, —Cl, and —Br;
R⁷ is selected from the group consisting of —CONHR²⁸ and —COOC(CH₃)₃; R¹¹ is selected from the group consisting of —H, —CH₃, —F, —Cl, —Br, —CN, —OCH₃, —CH₂OH, —SCH₃, cyclopropyl, —NO₂, —NHCCH₃O, —P(CH₃)₂O, —C(CH₃)₂R³⁷, —COOR¹⁸, —CF₂R³⁸, —SO₂R³⁹, —SC(CH₃)₃, 1,2-thiazol-5-yl, —SCF₃, —OCF₂R²⁷, pyridin-4-yl, 1-methyl-1H-pyrazol-3-yl, —CON(R¹⁶)CH₂R¹⁸, —COC(R²⁷)₃, 1-methylpiperidin-4-yl, 1-methyl-1,2,3,6-tetrahydropyridin-4-yl, 1,5-dimethyl-1H-pyrazol-4-yl, (cyclopentylmethyl)sulfanyl, 1-(pyridin-4-yl)ethoxy, 5H,6H,7H-cyclopenta[b]pyridin-7-yloxy,

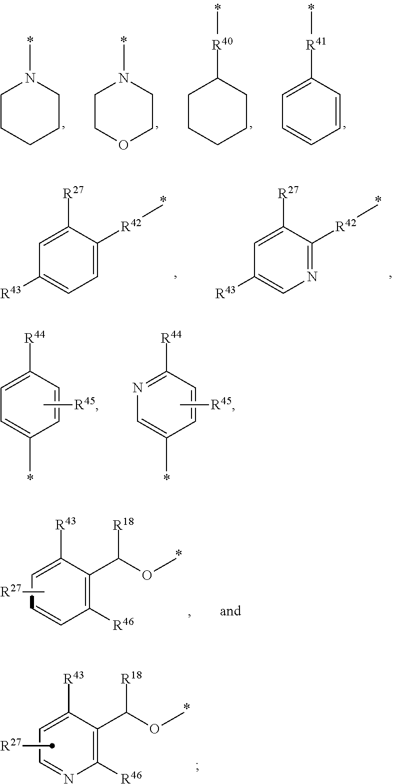

R¹² is selected from the group consisting of —H, —CH₃, —F, and —CF₃;
R¹⁶ is selected from the group consisting of —H, —CH₃, and —CH₂CH₃;
R¹⁸ is selected from the group consisting of —H and —CH₃;
R²⁷ is selected from the group consisting of —H and —F;
R²⁸ is selected from the group consisting of —CH₂CH₃, —CH(CH₃)₂, and cyclobutyl;
R³⁷ is selected from the group consisting of —H and —OH;
R³⁸ is selected from the group consisting of —H, —CH₃, and —F;
R³⁹ is selected from the group consisting of —CH₃, —NH₂, —NHCH₃, —N(CH₃)₂, morpholin-4-yl, and 4-methylpiperazin-1-yl;
R⁴⁰ is selected from the group consisting of —NHCO—, —CHCH₃S—, —CH₂SO—, —CH₂SO₂—, and **—N—CH₂—*, **—O—CH₂—*, and **—S—CH₂—*;
R⁴¹ is selected from the group consisting of —CH₂S—, —SO—, —NHCO—, —CHCH₃S—, —O—, and —S—;
R⁴² is selected from the group consisting of —CH₂O—, —CHCH₃O—, and —CHCH₃NHCO—;
R⁴³ is selected from the group consisting of —H and —Cl;
R⁴⁴ is selected from the group consisting of —H, —CH₃, —CCH₃O, —SCH₃O, —SO₂R⁶⁴, and dimethoxyphosphoryl;
R⁴⁵ is selected from the group consisting of —H, —CH₃, —F, —OCH₃, and —SCH₃O₂;
R⁴⁶ is selected from the group consisting of —H, —F, and —Cl;
R⁶⁴ is selected from the group consisting of —CH₃, —NH₂, —NHCH₃, cyclopropyl, and —N(CH₃)₂;
or a stereoisomer, a tautomer, an N-oxide, a hydrate, a solvate, or a salt thereof, or a mixture of same.

### Embodiment Q

A compound according to embodiment M of formula (I-6)

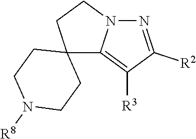

R² is selected from the group consisting of 4-amino-1H-imidazo[4,5-c]pyridin-7-yl, 6-amino-5-cyclohexylmethanesulfonyl-1-oxidopyridin-1-ium-3-yl, and

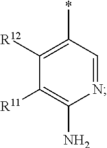

R³ is selected from the group consisting of —H, —Cl, and —Br;
R⁸ is selected from the group consisting of —CONHR²⁹, (1H-imidazol-2-yl)methyl, and —COOC(CH₃)₃;
R¹¹ is selected from the group consisting of —H, —CH₃, —F, —Cl, —Br, —CN, —OCH₃, —CH₂OH, —SCH₃, cyclopropyl, —NO₂, —NHCCH₃O, —P(CH₃)₂O, —C(CH₃)₂R³⁷, —COOR¹⁸, —CF₂R³⁸, —SO₂R³⁹, —SC(CH₃)₃, 1,2-thiazol-5-yl, —SCF₃, —OCF₂R²⁷, pyridin-4-yl, 1-methyl-1H-pyrazol-3-yl, —CON(R¹⁶)CH₂R¹⁸, —COC(R²⁷)₃, 1-methylpiperidin-4-yl, 1-methyl-1,2,3,6-tetrahydropyridin-4-yl, 1,5-dimethyl-1H-pyrazol-4-yl, (cyclopentylmethyl)sulfanyl, 1-(pyridin-4-yl)ethoxy, 5H,6H,7H-cyclopenta[b]pyridin-7-yloxy,

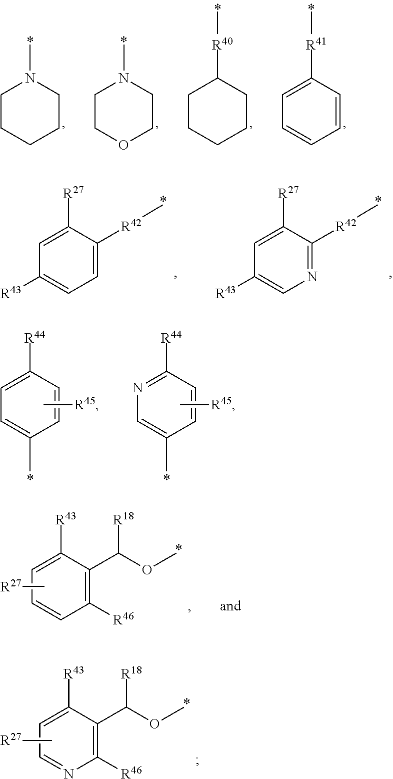

R¹² is selected from the group consisting of —H, —CH₃, —F, and —CF₃;
R¹⁶ is selected from the group consisting of —H, —CH₃, and —CH₂CH₃;
R¹⁸ is selected from the group consisting of —H and —CH₃;
R²⁷ is selected from the group consisting of —H and —F;
R²⁹ is selected from the group consisting of —CH₂CH₃, —CH(CH₃)₂, cyclobutyl, and

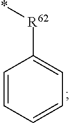

R³⁷ is selected from the group consisting of —H and —OH;
R³⁸ is selected from the group consisting of —H, —CH₃, and —F;
R³⁹ is selected from the group consisting of —CH₃, —NH₂, —NHCH₃, —N(CH₃)₂, morpholin-4-yl, and 4-methylpiperazin-1-yl;
R⁴⁰ is selected from the group consisting of —NHCO—, —CHCH₃S—, —CH₂SO—, —CH₂SO₂—, and **—N—CH₂—*, **—O—CH₂—*, and **—S—CH₂—*;
R⁴¹ is selected from the group consisting of —CH₂S—, —SO—, —NHCO—, —CHCH₃S—, —O—, and —S—;
R⁴² is selected from the group consisting of —CH₂O—, —CHCH₃O—, and —CHCH₃NHCO—;
R⁴³ is selected from the group consisting of —H and —Cl;
R⁴⁴ is selected from the group consisting of —H, —CH₃, —CCH₃O, —SCH₃O, —SO₂R⁶⁴, and dimethoxyphosphoryl;
R⁴⁵ is selected from the group consisting of —H, —CH₃, —F, —OCH₃, and —SCH₃O₂;
R⁴⁶ is selected from the group consisting of —H, —F, and —Cl;
R⁶² is selected from the group consisting of —CH₂— and

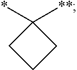

R⁶⁴ is selected from the group consisting of —CH₃, —NH₂, —NHCH₃, cyclopropyl, and —N(CH₃)₂;
or a stereoisomer, a tautomer, an N-oxide, a hydrate, a solvate, or a salt thereof, or a mixture of same.

### Embodiment R

A compound according to embodiment M of formula (I-7)

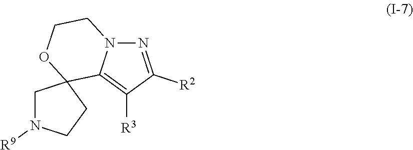

wherein
R² is selected from the group consisting of 4-amino-1H-imidazo[4,5-c]pyridin-7-yl, 6-amino-5-cyclohexylmethanesulfonyl-1-oxidopyridin-1-ium-3-yl, and

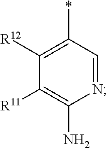

R³ is selected from the group consisting of —H, —Cl, and —Br;
R⁹ is selected from the group consisting of —CONHR³⁰, (1H-imidazol-2-yl)methyl, and —COOC(CH₃)₃;
R¹¹ is selected from the group consisting of —H, —CH₃, —F, —Cl, —Br, —CN, —OCH₃, —CH₂OH, —SCH₃, cyclopropyl, —NO₂, —NHCCH₃O, —P(CH₃)₂O, —C(CH₃)₂R³⁷, —COOR¹⁸, —CF₂R³⁸, —SO₂R³⁹, —SC(CH₃)₃, 1,2-thiazol-5-yl, —SCF₃, —OCF₂R²⁷, pyridin-4-yl, 1-methyl-1H-pyrazol-3-yl, —CON(R¹⁶)CH₂R¹⁸, —COC(R²⁷)₃, 1-methylpiperidin-4-yl, 1-methyl-1,2,3,6-tetrahydropyridin-4-yl, 1,5-dimethyl-1H-pyrazol-4-yl, (cyclopentylmethyl)sulfanyl, 1-(pyridin-4-yl)ethoxy, 5H,6H,7H-cyclopenta[b]pyridin-7-yloxy,

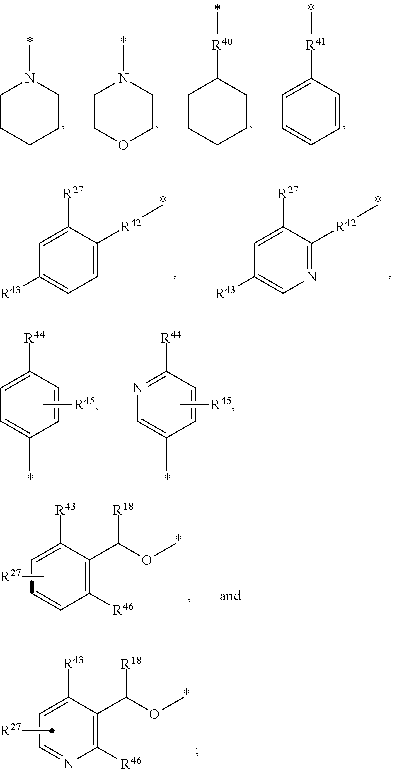

R¹² is selected from the group consisting of —H, —CH₃, —F, and —CF₃;
R¹⁶ is selected from the group consisting of —H, —CH₃, and —CH₂CH₃;
R¹⁸ is selected from the group consisting of —H and —CH₃;
R²⁷ is selected from the group consisting of —H and —F;
R³⁰ is selected from the group consisting of —CH₂CH₃, cyclobutyl, —C(CH₃)₂R⁶³, 1-(5-fluoropyridin-3-yl)ethyl,

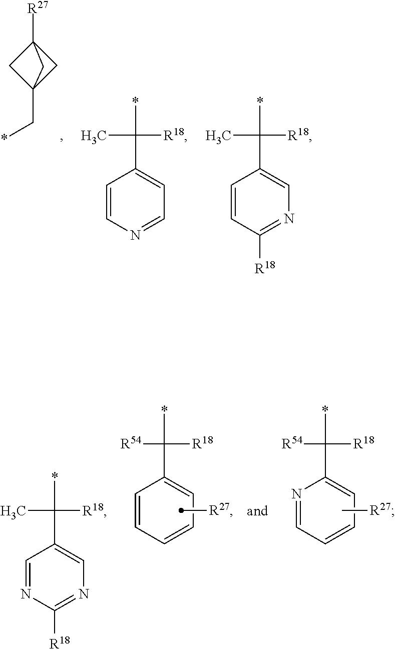

R³⁷ is selected from the group consisting of —H and —OH;
R³⁸ is selected from the group consisting of —H, —CH₃, and —F;
R³⁹ is selected from the group consisting of —CH₃, —NH₂, —NHCH₃, —N(CH₃)₂, morpholin-4-yl, and 4-methylpiperazin-1-yl;
R⁴⁰ is selected from the group consisting of —NHCO—, —CHCH₃S—, —CH₂SO—, —CH₂SO₂—, and **—N—CH₂—*, **—O—CH₂—*, and **—S—CH₂—*;
R⁴¹ is selected from the group consisting of —CH₂S—, —SO—, —NHCO—, —CHCH₃S—, —O—, and —S—;
R⁴² is selected from the group consisting of —CH₂O—, —CHCH₃O—, and —CHCH₃NHCO—;
R⁴³ is selected from the group consisting of —H and —Cl;
R⁴⁴ is selected from the group consisting of —H, —CH₃, —CCH₃O, —SCH₃O, —SO₂R⁶⁴, and dimethoxyphosphoryl;
R⁴⁵ is selected from the group consisting of —H, —CH₃, —F, —OCH₃, and —SCH₃O₂;
R⁴⁶ is selected from the group consisting of —H, —F, and —Cl;
R⁵⁴ is selected from the group consisting of —H, —CH₃, —CH₂CH₃, and —CH₂OCH₃;
R⁶³ is selected from the group consisting of —H, 1-methyl-1H-1,2,3-triazol-4-yl, and 3-chloropyridin-4-yl;
R⁶⁴ is selected from the group consisting of —CH₃, —NH₂, —NHCH₃, cyclopropyl, and —N(CH₃)₂;
or a stereoisomer, a tautomer, an N-oxide, a hydrate, a solvate, or a salt thereof, or a mixture of same.

### Embodiment 5

A compound according to embodiment M of formula (I-8)

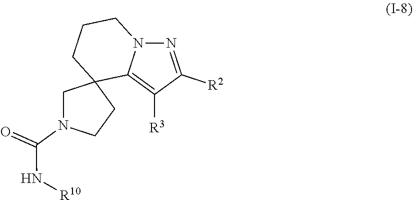

wherein
R² is selected from the group consisting of 4-amino-1H-imidazo[4,5-c]pyridin-7-yl, 6-amino-5-cyclohexylmethanesulfonyl-1-oxidopyridin-1-ium-3-yl, and

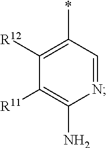

R³ is selected from the group consisting of —H, —Cl, and —Br;
R¹⁰ is selected from the group consisting of —CH₂CH₃, {1-methyl-2-oxabicyclo[2.1.1]hexan-4-yl}methyl, 1-(1,3,4-trimethyl-1H-pyrazol-5-yl)ethyl,

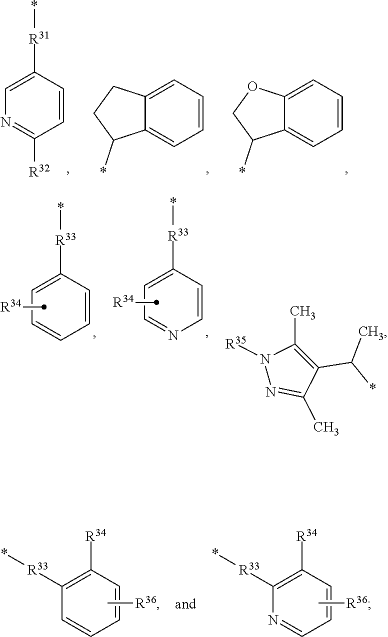

R¹¹ is selected from the group consisting of —H, —CH₃, —F, —Cl, —Br, —CN, —OCH₃, —CH₂OH, —SCH₃, cyclopropyl, —NO₂, —NHCCH₃O, —P(CH₃)₂O, —C(CH₃)₂R³⁷, —COOR¹⁸, —CF₂R³⁸, —SO₂R³⁹, —SC(CH₃)₃, 1,2-thiazol-5-yl, —SCF₃, —OCF₂R²⁷, pyridin-4-yl, 1-methyl-1H-pyrazol-3-yl, —CON(R¹⁶)CH₂R¹⁸, —COC(R²⁷)₃, 1-methylpiperidin-4-yl, 1-methyl-1,2,3,6-tetrahydropyridin-4-yl, 1,5-dimethyl-1H-pyrazol-4-yl, (cyclopentylmethyl)sulfanyl, 1-(pyridin-4-yl)ethoxy, 5H,6H,7H-cyclopenta[b]pyridin-7-yloxy,

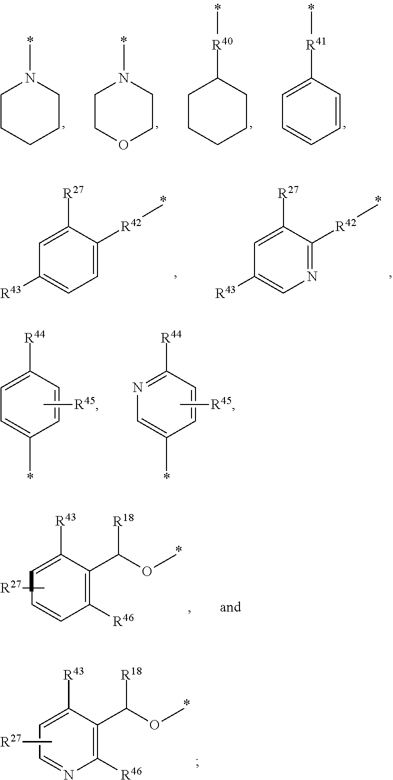

R¹² is selected from the group consisting of —H, —CH₃, —F, and —CF₃;
R¹⁶ is selected from the group consisting of —H, —CH₃, and —CH₂CH₃;
R¹⁸ is selected from the group consisting of —H and —CH₃;
R²⁷ is selected from the group consisting of —H and —F;
R³¹ is selected from the group consisting of —CHCH₃— and

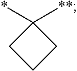

R³² is selected from the group consisting of —H and —CN;
R³³ is selected from the group consisting of —CHCH₃—, —C(CH₃)₂—, and

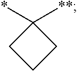

R³⁴ is selected from the group consisting of —H, —F, —Cl, and —CN;
R³⁵ is selected from the group consisting of —CH₃ and —CH₂CH₃;
R³⁶ is selected from the group consisting of —H, —F, and —CN;
R³⁷ is selected from the group consisting of —H and —OH;
R³⁸ is selected from the group consisting of —H, —CH₃, and —F;
R³⁹ is selected from the group consisting of —CH₃, —NH₂, —NHCH₃, —N(CH₃)₂, morpholin-4-yl, and 4-methylpiperazin-1-yl;
R⁴⁰ is selected from the group consisting of —NHCO—, —CHCH₃S—, —CH₂SO—, —CH₂SO₂—, and **—N—CH₂—*, **—O—CH₂—*, and **—S—CH₂—*;
R⁴¹ is selected from the group consisting of —CH₂S—, —SO—, —NHCO—, —CHCH₃S—, —O—, and —S—;
R⁴² is selected from the group consisting of —CH₂O—, —CHCH₃O—, and —CHCH₃NHCO—;
R⁴³ is selected from the group consisting of —H and —Cl;
R⁴⁴ is selected from the group consisting of —H, —CH₃, —CCH₃O, —SCH₃O, —SO₂R⁶⁴, and dimethoxyphosphoryl;
R⁴⁵ is selected from the group consisting of —H, —CH₃, —F, —OCH₃, and —SCH₃O₂;
R⁴⁶ is selected from the group consisting of —H, —F, and —Cl;
R⁶⁴ is selected from the group consisting of —CH₃, —NH₂, —NHCH₃, cyclopropyl, and —N(CH₃)₂;
or a stereoisomer, a tautomer, an N-oxide, a hydrate, a solvate, or a salt thereof, or a mixture of same.

### Embodiment T

A compound according to embodiment M of formula (I-9)

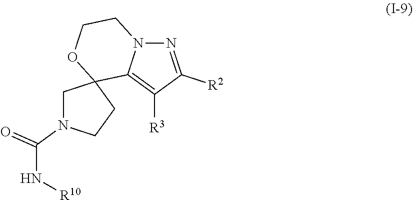

wherein
R² is selected from the group consisting of 4-amino-1H-imidazo[4,5-c]pyridin-7-yl, 6-amino-5-cyclohexylmethanesulfonyl-1-oxidopyridin-1-ium-3-yl, and

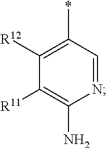

R³ is selected from the group consisting of —H, —Cl, and —Br;
R¹⁰ is selected from the group consisting of —CH₂CH₃, {1-methyl-2-oxabicyclo[2.1.1]hexan-4-yl}methyl, 1-(1,3,4-trimethyl-1H-pyrazol-5-yl)ethyl,

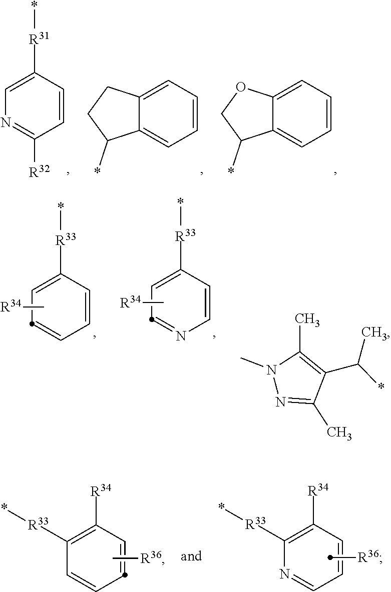

R¹¹ is selected from the group consisting of —H, —CH₃, —F, —Cl, —Br, —CN, —OCH₃, —CH₂OH, —SCH₃, cyclopropyl, —NO₂, —NHCCH₃O, —P(CH₃)₂O, —C(CH₃)₂R³⁷, —COOR¹⁸, —CF₂R³⁸, —SO₂R³⁹, —SC(CH₃)₃, 1,2-thiazol-5-yl, —SCF₃, —OCF₂R²⁷, pyridin-4-yl, 1-methyl-1H-pyrazol-3-yl, —CON(R¹⁶)CH₂R¹⁸, —COC(R²⁷)₃, 1-methylpiperidin-4-yl, 1-methyl-1,2,3,6-tetrahydropyridin-4-yl, 1,5-dimethyl-1H-pyrazol-4-yl, (cyclopentylmethyl)sulfanyl, 1-(pyridin-4-yl)ethoxy, 5H,6H,7H-cyclopenta[b]pyridin-7-yloxy,

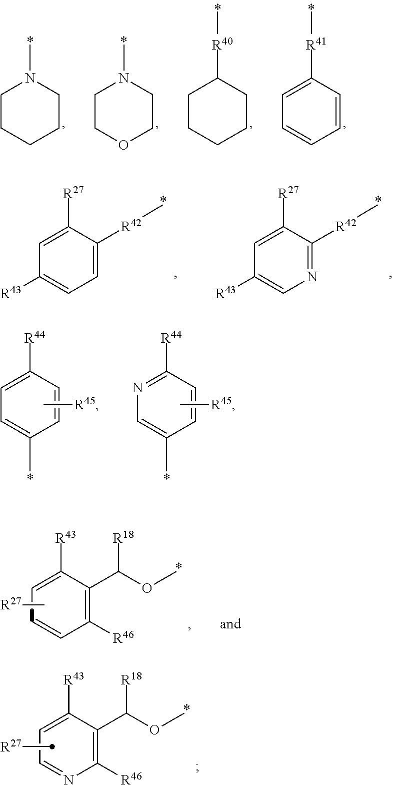

R¹² is selected from the group consisting of —H, —CH₃, —F, and —CF₃;
R¹⁶ is selected from the group consisting of —H, —CH₃, and —CH₂CH₃;
R¹⁸ is selected from the group consisting of —H and —CH₃;
R²⁷ is selected from the group consisting of —H and —F;
R³¹ is selected from the group consisting of —CHCH₃— and

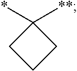

R³² is selected from the group consisting of —H and —CN;
R³³ is selected from the group consisting of —CHCH₃—, —C(CH₃)₂—, and

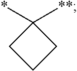

R³⁴ is selected from the group consisting of —H, —F, —Cl, and —CN;
R³⁵ is selected from the group consisting of —CH₃ and —CH₂CH₃;
R³⁶ is selected from the group consisting of —H, —F, and —CN;
R³⁷ is selected from the group consisting of —H and —OH;
R³⁸ is selected from the group consisting of —H, —CH₃, and —F;
R³⁹ is selected from the group consisting of —CH₃, —NH₂, —NHCH₃, —N(CH₃)₂, morpholin-4-yl, and 4-methylpiperazin-1-yl;
R⁴⁰ is selected from the group consisting of —NHCO—, —CHCH₃S—, —CH₂SO—, —CH₂SO₂—, and **—N—CH₂—*, **—O—CH₂—*, and **—S—CH₂—*;
R⁴¹ is selected from the group consisting of —CH₂S—, —SO—, —NHCO—, —CHCH₃S—, —O—, and —S—;
R⁴² is selected from the group consisting of —CH₂O—, —CHCH₃O—, and —CHCH₃NHCO—;
R⁴³ is selected from the group consisting of —H and —Cl;
R⁴⁴ is selected from the group consisting of —H, —CH₃, —CCH₃O, —SCH₃O, —SO₂R⁶⁴, and dimethoxyphosphoryl;
R⁴⁵ is selected from the group consisting of —H, —CH₃, —F, —OCH₃, and —SCH₃O₂;
R⁴⁶ is selected from the group consisting of —H, —F, and —Cl;
R⁶⁴ is selected from the group consisting of —CH₃, —NH₂, —NHCH₃, cyclopropyl, and —N(CH₃)₂;
or a stereoisomer, a tautomer, an N-oxide, a hydrate, a solvate, or a salt thereof, or a mixture of same.

### Embodiment U

A compound according to embodiment M of formula (I-10)

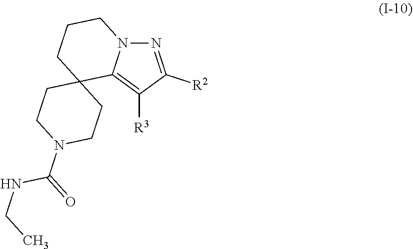

wherein
R² is selected from the group consisting of 4-amino-1H-imidazo[4,5-c]pyridin-7-yl, 6-amino-5-cyclohexylmethanesulfonyl-1-oxidopyridin-1-ium-3-yl, and

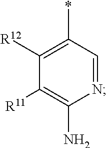

R³ is selected from the group consisting of —H, —Cl, and —Br;
R¹¹ is selected from the group consisting of —H, —CH₃, —F, —Cl, —Br, —CN, —OCH₃, —CH₂OH, —SCH₃, cyclopropyl, —NO₂, —NHCCH₃O, —P(CH₃)₂O, —C(CH₃)₂R³⁷, —COOR¹⁸, —CF₂R³⁸, —SO₂R³⁹, —SC(CH₃)₃, 1,2-thiazol-5-yl, —SCF₃, —OCF₂R²⁷, pyridin-4-yl, 1-methyl-1H-pyrazol-3-yl, —CON(R¹⁶)CH₂R¹⁸, —COC(R²⁷)₃, 1-methylpiperidin-4-yl, 1-methyl-1,2,3,6-tetrahydropyridin-4-yl, 1,5-dimethyl-1H-pyrazol-4-yl, (cyclopentylmethyl)sulfanyl, 1-(pyridin-4-yl)ethoxy, 5H,6H,7H-cyclopenta[b]pyridin-7-yloxy,

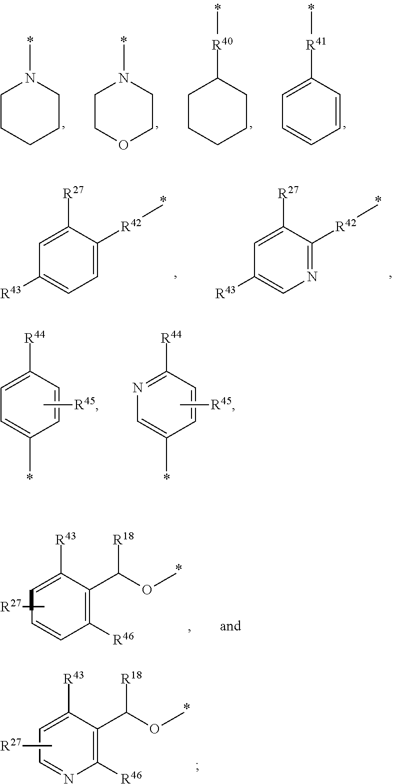

R¹² is selected from the group consisting of —H, —CH₃, —F, and —CF₃;
R¹⁶ is selected from the group consisting of —H, —CH₃, and —CH₂CH₃;
R¹⁸ is selected from the group consisting of —H and —CH₃;
R²⁷ is selected from the group consisting of —H and —F;
R³⁷ is selected from the group consisting of —H and —OH;
R³⁸ is selected from the group consisting of —H, —CH₃, and —F;
R³⁹ is selected from the group consisting of —CH₃, —NH₂, —NHCH₃, —N(CH₃)₂, morpholin-4-yl, and 4-methylpiperazin-1-yl;
R⁴⁰ is selected from the group consisting of —NHCO—, —CHCH₃S—, —CH₂SO—, —CH₂SO₂—, and **—N—CH₂—*, **—O—CH₂—*, and **—S—CH₂—*;
R⁴¹ is selected from the group consisting of —CH₂S—, —SO—, —NHCO—, —CHCH₃S—, —O—, and —S—;
R⁴² is selected from the group consisting of —CH₂O—, —CHCH₃O—, and —CHCH₃NHCO—;
R⁴³ is selected from the group consisting of —H and —Cl;
R⁴⁴ is selected from the group consisting of —H, —CH₃, —CCH₃O, —SCH₃O, —SO₂R⁶⁴, and dimethoxyphosphoryl;
R⁴⁵ is selected from the group consisting of —H, —CH₃, —F, —OCH₃, and —SCH₃O₂;
R⁴⁶ is selected from the group consisting of —H, —F, and —Cl;
R⁶⁴ is selected from the group consisting of —CH₃, —NH₂, —NHCH₃, cyclopropyl, and —N(CH₃)₂;
or a stereoisomer, a tautomer, an N-oxide, a hydrate, a solvate, or a salt thereof, or a mixture of same.

The compounds of general formula (I) of the present invention can be converted to any salt, preferably pharmaceutically acceptable salts, as described herein, by any method which is known to the person skilled in the art. Similarly, any salt of a compound of general formula (I) of the present invention can be converted into the free compound, by any method which is known to the person skilled in the art.

Compounds of general formula (I) of the present invention demonstrate a valuable pharmacological spectrum of action, which could not have been predicted. Compounds of the present invention have surprisingly been found to effectively inhibit MAP4K1 and it is possible therefore that said compounds be used for the treatment or prophylaxis of diseases, preferably cancer or conditions with dysregulated immune responses or other disorders associated with aberrant MAP4K1 signaling, in humans and animals.

Disorders and conditions particularly suitable for treatment with an MAP4K1 inhibitor of the present invention are liquid and solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias.

Examples of breast cancers include, but are not limited to, triple negative breast cancer, invasive ductal carcinoma, invasive lobular carcinoma, ductal carcinoma in situ, and lobular carcinoma in situ. Examples of cancers of the respiratory tract include, but are not limited to, small-cell and non-small-cell lung carcinoma, as well as bronchial adenoma and pleuropulmonary blastoma.

Examples of brain cancers include, but are not limited to, brain stem and hypophtalmic glioma, cerebellar and cerebral astrocytoma, glioblastoma, medulloblastoma, ependymoma, as well as neuroectodermal and pineal tumour.

Tumours of the male reproductive organs include, but are not limited to, prostate and testicular cancer.

Tumours of the female reproductive organs include, but are not limited to, endometrial, cervical, ovarian, vaginal, and vulvar cancer, as well as sarcoma of the uterus.

Examples of ovarian cancer include, but are not limited to serous tumour, endometrioid tumour, mucinous cystadenocarcinoma, granulosa cell tumour, Sertoli-Leydig cell tumour and arrhenoblastoma.

Examples of cervical cancer include, but are not limited to squamous cell carcinoma, adenocarcinoma, adenosquamous carcinoma, small cell carcinoma, neuroendocrine tumour, glassy cell carcinoma and villoglandular adenocarcinoma.

Tumours of the digestive tract include, but are not limited to, anal, colon, colorectal, esophageal, gallbladder, gastric, pancreatic, rectal, small-intestine, and salivary gland cancers.

Examples of esophageal cancer include, but are not limited to esophageal cell carcinomas and adenocarcinomas, as well as squamous cell carcinomas, leiomyosarcoma, malignant melanoma, rhabdomyosarcoma and lymphoma.

Examples of gastric cancer include, but are not limited to intestinal type and diffuse type gastric adenocarcinoma.

Examples of pancreatic cancer include, but are not limited to ductal adenocarcinoma, adenosquamous carcinomas and pancreatic endocrine tumours.

Tumours of the urinary tract include, but are not limited to, bladder, penile, kidney, renal pelvis, ureter, urethral and human papillary renal cancers.

Examples of kidney cancer include, but are not limited to renal cell carcinoma, urothelial cell carcinoma, juxtaglomerular cell tumour (reninoma), angiomyolipoma, renal oncocytoma, Bellini duct carcinoma, clear-cell sarcoma of the kidney, mesoblastic nephroma and Wilms' tumour.

Examples of bladder cancer include, but are not limited to transitional cell carcinoma, squamous cell carcinoma, adenocarcinoma, sarcoma and small cell carcinoma.

Eye cancers include, but are not limited to, intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to, squamous cell carcinoma, Kaposi's sarcoma, malignant melanoma, Merkel cell skin cancer, and non-melanoma skin cancer.

Head-and-neck cancers include, but are not limited to, squamous cell cancer of the head and neck, laryngeal, hypopharyngeal, nasopharyngeal, oropharyngeal cancer, salivary gland cancer, lip and oral cavity cancer and squamous cell.

Lymphomas include, but are not limited to, AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to, sarcoma of the soft tissue, osteosarcoma, malignant fibrous histiocytoma, lymphosarcoma, and rhabdomyosarcoma.

Leukemias include, but are not limited to, acute myeloid leukemia, acute lymphoblastic leukemia, chronic lymphocytic leukemia, chronic myelogenous leukemia, and hairy cell leukemia.

The term “treating” or “treatment” as stated throughout this document is used conventionally, for example the management or care of a subject for the purpose of combating, alleviating, reducing, relieving, improving the condition of a disease or disorder, such as a carcinoma.

The compounds of the present invention can be used in particular in therapy and prevention, i.e. prophylaxis, of tumour growth and metastases, especially in solid tumours of all indications and stages with or without pre-treatment of the tumour growth.

Generally, the use of chemotherapeutic agents and/or anti-cancer agents in combination with a compound or pharmaceutical composition of the present invention will serve to:
1. yield better efficacy in reducing the growth of a tumour or even eliminate the tumour as compared to administration of either agent alone,
2. provide for the administration of lesser amounts of the administered chemotherapeutic agents,
3. provide for a chemotherapeutic treatment that is well tolerated in the patient with fewer deleterious pharmacological complications than observed with single agent chemotherapies and certain other combined therapies,
4. provide for treating a broader spectrum of different cancer types in mammals, especially humans,
5. provide for a higher response rate among treated patients,
6. provide for a longer survival time among treated patients compared to standard chemotherapy treatments,
7. provide a longer time for tumour progression, and/or
8. yield efficacy and tolerability results at least as good as those of the agents used alone, compared to known instances where other cancer agent combinations produce antagonistic effects.

In addition, the compounds of general formula (I) of the present invention can also be used in combination with radiotherapy and/or surgical intervention.

In a further embodiment of the present invention, the compounds of general formula (I) of the present invention are used in combination with radiation: i.e. radiation treatment sensitizes cancers to anti-tumor immune responses by induction of tumor cell death and subsequent presentation of tumor neoantigens to tumor-reactive Tcells. As MAP4K1 inhibition is enhancing the antigen specific activation of T cells, the overall effect results in a much stronger cancer cell attack as compared to irradiation treatment alone.

Thus, the present invention also provides a method of killing a tumor, wherein conventional radiation therapy is employed previous to administering one or more of the compounds of the present invention.

The compounds of the present invention can be administered as the sole pharmaceutical agent or in combination with one or more other pharmaceutically active ingredients where the combination causes no unacceptable adverse effects. The present invention also covers such pharmaceutical combinations. For example, the compounds of the present invention can be combined with: ¹³¹I-chTNT, abarelix, abiraterone, aclarubicin, adalimumab, ado-trastuzumab emtansine, afatinib, aflibercept, aldesleukin, alectinib, alemtuzumab, alendronic acid, alitretinoin, alpharain, altretamine, amifostine, aminoglutethimide, hexyl aminolevulinate, amrubicin, amsacrine, anastrozole, ancestim, anethole dithiolethione, anetumab ravtansine, angiotensin II, antithrombin Ill, aprepitant, arcitumomab, arglabin, arsenic trioxide, asparaginase, atezolizumab, axitinib, azacitidine, basiliximab, belotecan, bendamustine, besilesomab, belinostat, bevacizumab, bexarotene, bicalutamide, bisantrene, bleomycin, blinatumomab, bortezomib, buserelin, bosutinib, brentuximab vedotin, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, cemiplimab, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, darolutamide, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, eculizumab, edrecolomab, elliptinium acetate, elotuzumab, eltrombopag, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, 1-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, interferon alfa, interferon beta, interferon gamma, iobitridol, iobenguane (¹²³I), iomeprol, ipilimumab, irinotecan, Itraconazole, ixabepilone, ixazomib, lanreotide, lansoprazole, lapatinib, larotrectinib, lasocholine, lenalidomide, lenvatinib, lenograstim, lentinan, letrozole, leuprorelin, levamisole, levonorgestrel, levothyroxine sodium, lisuride, lobaplatin, lomustine, lonidamine, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, nabilone, nabiximols, nafarelin, naloxone+pentazocine, naltrexone, nartograstim, necitumumab, nedaplatin, nelarabine, neridronic acid, netupitant/palonosetron, nivolumab, pentetreotide, nilotinib, nilutamide, nimorazole, nimotuzumab, nimustine, nintedanib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pembrolizumab, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, risedronic acid, rhenium-186 etidronate, rituximab, rogaratinib, rolapitant, romidepsin, romiplostim, romurtide, roniciclib, samarium (153Sm) lexidronam, sargramostim, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (^{99m}Tc) nofetumomab merpentan, ^{99m}Tc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tisagenlecleucel, tislelizumab, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

The compounds of the invention can further be combined with other reagents targeting the immune system, such as immune checkpoint inhibitors, e.g. aPD-1/-L1 axis antagonists. PD-1, along with its ligands PD-L1 and PD-L2, function as negative regulators of T cell activation. MAP4K1 suppresses immune cell function. PD-L1 is overexpressed in many cancers and overexpression of PD-1 often occurs concomitantly in tumor infiltrating T cells. Thus results in attenuation of T cell activation and evasion of immune surveillance, which contributes to impaired antitumor immune responses. (Keir M E et al. (2008) Annu. Rev. Immunol. 26:677).

In accordance with a further aspect, the present invention covers combinations comprising one or more of the compounds of general formula (I), as described supra, or stereoisomers, tautomers, N-oxides, hydrates, solvates, and salts thereof, particularly pharmaceutically acceptable salts thereof, or mixtures of same, and one or more immune checkpoint inhibitors.

In a further embodiment the immune checkpoint inhibitor is a aPD-1/-L1 axis antagonist.

A further use of the compounds of the invention is the combination with chimeric antigen receptor T cells (CAR-T cells) such as Axicabtagen-Ciloleucel or Tisagenlecleucel. The activity of CAR-T cells can be suppressed by the tumor micro environment (TME), which supposedly can be overcome by MAP4K1 inhibition.

In accordance with a further aspect, the present invention covers compounds of general formula (I), as described herein, or stereoisomers, tautomers, N-oxides, hydrates, solvates, and salts thereof, particularly pharmaceutically acceptable salts thereof, or mixtures of same, for use in the expansion of T cells including CAR-T cells, CAR-NKT cells or CAR-NK cells and tumor infiltrated lymphocytes ex-vivo.

Hence, the present invention also relates to the use of the compounds according to the invention for the expansion of T cells, including CAR-T cell, CAR-NKT cells or CAR-NK cells and tumor infiltrated lymphocytes, ex-vivo.

The present invention also comprises an ex-vivo method for the expansion of T cells, including CAR-T cells, CAR-NKT cells or CAR-NK cells and tumor infiltrated lymphocytes, contacting said T cells with compounds according to the invention.

In addition, the inventive compounds can also be used as a therapeutic in a variety of other disorders wherein MAP4K1 is involved such as, cardiovascular and lung diseases. Accordingly, the compounds according to the invention are suitable for the treatment and/or prophylaxis in particular of cardiovascular, inflammatory and fibrotic disorders and of renal disorders, in particular of acute and chronic renal insufficiency, and also of acute and chronic renal failure.

Accordingly, the compounds according to the invention can be used in medicaments for the treatment and/or prophylaxis of cardiovascular, inflammatory and fibrotic disorders, renal disorders, in particular of acute and chronic renal insufficiency, and also of acute and chronic renal failure.

For the purpose of the present invention the term renal insufficiency comprises both acute and chronic manifestations of renal insufficiency, and also underlying or related renal disorders such as diabetic and non-diabetic nephropathies, hypertensive nephropathies, ischaemic renal disorders, renal hypoperfusion, intradialytic hypotension, obstructive uropathy, renal stenoses, glomerulopathies, glomerulonephritis (such as, for example, primary glomerulonephritides; minimal change glomerulonephritis (lipoidnephrosis); membranous glomerulonephritis; focal segmental glomerulosclerosis (FSGS); membrane-proliferative glomerulonephritis; crescentic glomerulonephritis; mesangioproliferative glomerulonephritis (IgA nephritis, Berger's disease); post-infectious glomerulonephritis; secondary glomerulonephritides: diabetes mellitus, lupus erythematosus, amyloidosis, Goodpasture syndrome, Wegener granulomatosis, Henoch-Schönlein purpura, microscopic polyangiitis, acute glomerulonephritis, pyelonephritis (for example as a result of: urolithiasis, benign prostate hyperplasia, diabetes, malformations, abuse of analgesics, Crohn's disease), glomerulosclerosis, arteriolonecrose of the kidney, tubulointerstitial diseases, nephropathic disorders such as primary and congenital or acquired renal disorder, Alport syndrome, nephritis, immunological kidney disorders such as kidney transplant rejection and immunocomplex-induced renal disorders, nephropathy induced by toxic substances, nephropathy induced by contrast agents, diabetic and non-diabetic nephropathy, renal cysts, nephrosclerosis, hypertensive nephrosclerosis and nephrotic syndrome which can be characterized diagnostically, for example by abnormally reduced creatinine and/or water excretion, abnormally elevated blood concentrations of urea, nitrogen, potassium and/or creatinine, altered activity of renal enzymes, for example glutamyl synthetase, altered urine osmolarity or urine volume, elevated microalbuminuria, macroalbuminuria, lesions on glomerulae and arterioles, tubular dilatation, hyperphosphataemia and/or the need for dialysis. The present invention also comprises the use of the compounds according to the invention for the treatment and/or prophylaxis of sequelae of renal insufficiency, for example pulmonary oedema, heart failure, uremia, anemia, electrolyte disturbances (for example hypercalemia, hyponatremia) and disturbances in bone and carbohydrate metabolism.

The present invention also comprises the use of the compounds according to the invention for the treatment and/or prevention of sequelae of renal insufficiency, for example pulmonary oedema, heart failure, uraemia, anaemia, electrolyte disturbances (for example hyperkalaemia, hyponatraemia) and disturbances in bone and carbohydrate metabolism.

The compounds according to the invention are further suitable for the treatment and/or prevention of polycystic kidney disease (PCKD) and of the syndrome of inappropriate ADH secretion (SIADH).

Furthermore, the compounds according to the invention are also suitable for the treatment and/or prophylaxis of metabolic syndrome, hypertension, resistant hypertension, acute and chronic heart failure, coronary heart disease, stable and unstable angina pectoris, peripheral and cardiac vascular disorders, arrhythmias, atrial and ventricular arrhythmias and impaired conduction, for example atrioventricular blocks degrees I-Ill (AB block I-Ill), supraventricular tachyarrhythmia, atrial fibrillation, atrial flutter, ventricular fibrillation, ventricular flutter, ventricular tachyarrhythmia, Torsade de pointes tachycardia, atrial and ventricular extrasystoles, AV-junctional extrasystoles, sick sinus syndrome, syncopes, AV-nodal re-entry tachycardia, Wolff-Parkinson-White syndrome, of acute coronary syndrome (ACS), autoimmune cardiac disorders (pericarditis, endocarditis, valvolitis, aortitis, cardiomyopathies), shock such as cardiogenic shock, septic shock and anaphylactic shock, aneurysms, boxer cardiomyopathy (premature ventricular contreaction (PVC)), for treatment and/or prophylaxis of thromboembolic disorders and ischaemias such as myocardial ischaemia, myocardial infarction, stroke, cardiac hypertrophy, transient and ischaemic attacks, preeclampsia, inflammatory cardiovascular disorders, spasms of the coronary arteries and peripheral arteries, oedema formation, for example pulmonary oedema, cerebral oedema, renal oedema or oedema caused by heart failure, peripheral circulatory disturbances, reperfusion damage, arterial and venous thromboses, myocardial insufficiency, endothelial dysfunction, to prevent restenoses, for example after thrombolysis therapies, percutaneous transluminal angioplasties (PTA), transluminal coronary angioplasties (PTCA), heart transplants and bypass operations, and also micro- and macrovascular damage (vasculitis), increased levels of fibrinogen and of low-density lipoprotein (LDL) and increased concentrations of plasminogen activator inhibitor 1 (PAI-1), and also for treatment and/or prophylaxis of erectile dysfunction and female sexual dysfunction.

In addition, the compounds according to the invention are also suitable for treatment and/or prophylaxis of asthmatic disorders, pulmonary arterial hypertension (PAH) and other forms of pulmonary hypertension (PH) including left-heart disease, HIV, sickle cell anaemia, thromboembolisms (CTEPH), sarcoidosis, COPD or pulmonary fibrosis-associated pulmonary hypertension, chronic-obstructive pulmonary disease (COPD), acute respiratory distress syndrome (ARDS), acute lung injury (ALI), alpha-1-antitrypsin deficiency (AATD), pulmonary fibrosis, pulmonary emphysema (for example pulmonary emphysema induced by cigarette smoke) and cystic fibrosis (CF).

The compounds described in the present invention are also active compounds for control of central nervous system disorders characterized by disturbances of the NO/cGMP system. They are suitable in particular for improving perception, concentration, learning or memory after cognitive impairments like those occurring in particular in association with situations/diseases/syndromes such as mild cognitive impairment, age-associated learning and memory impairments, age-associated memory losses, vascular dementia, craniocerebral trauma, stroke, dementia occurring after strokes (post stroke dementia), post-traumatic craniocerebral trauma, general concentration impairments, concentration impairments in children with learning and memory problems, Alzheimer's disease, Lewy body dementia, dementia with degeneration of the frontal lobes including Pick's syndrome, Parkinson's disease, progressive dementia with corticobasal degeneration, amyolateral sclerosis (ALS), Huntington's disease, demyelinization, multiple sclerosis, thalamic degeneration, Creutzfeld-Jacob dementia, HIV dementia, schizophrenia with dementia or Korsakoff's psychosis. They are also suitable for treatment and/or prophylaxis of central nervous system disorders such as states of anxiety, tension and depression, CNS-related sexual dysfunctions and sleep disturbances, and for controlling pathological disturbances of the intake of food, stimulants and addictive substances.

The compounds according to the invention are furthermore also suitable for controlling cerebral blood flow and thus represent effective agents for controlling migraines. They are also suitable for the prophylaxis and control of sequelae of cerebral infarction (cerebral apoplexy) such as stroke, cerebral ischaemia and craniocerebral trauma. The compounds according to the invention can likewise be used for controlling states of pain and tinnitus.

In addition, the compounds according to the invention have anti-inflammatory action and can therefore be used as anti-inflammatory agents for treatment and/or prophylaxis of sepsis (SIRS), multiple organ failure (MODS, MOF), inflammatory disorders of the kidney, chronic intestinal inflammations (IBD, Crohn's disease, UC), pancreatitis, peritonitis, rheumatoid disorders, inflammatory skin disorders and inflammatory eye disorders.

Furthermore, the compounds according to the invention can also be used for treatment and/or prophylaxis of autoimmune diseases.

The compounds according to the invention are also suitable for treatment and/or prophylaxis of fibrotic disorders of the internal organs, for example the lung, the heart, the kidney, the bone marrow and in particular the liver, and also dermatological fibroses and fibrotic eye disorders. In the context of the present invention, the term fibrotic disorders includes in particular the following terms: hepatic fibrosis, cirrhosis of the liver, pulmonary fibrosis, endomyocardial fibrosis, nephropathy, glomerulonephritis, interstitial renal fibrosis, fibrotic damage resulting from diabetes, bone marrow fibrosis and similar fibrotic disorders, scleroderma, morphea, keloids, hypertrophic scarring (also following surgical procedures), naevi, diabetic retinopathy, proliferative vitroretinopathy and disorders of the connective tissue (for example sarcoidosis).

The compounds according to the invention are also suitable for controlling postoperative scarring, for example as a result of glaucoma operations.

The compounds according to the invention can also be used cosmetically for ageing and keratinized skin.

Moreover, the compounds according to the invention are suitable for treatment and/or prophylaxis of hepatitis, neoplasms, osteoporosis, glaucoma and gastroparesis.

The present invention further provides the use of the compounds according to the invention for treatment and/or prophylaxis of disorders, especially the disorders mentioned above.

The present invention further provides the use of the compounds according to the invention for the treatment and/or prophylaxis of chronic renal disorders, acute and chronic renal insufficiency, diabetic, inflammatory or hypertensive nephropaties, fibrotic disorders, cardiac insufficiency, angina pectoris, hypertension, pulmonary hypertension, ischemias, vascular disorders, thromboembolic disorders, arteriosclerosis, sickle cell anemia, erectile dysfunction, benign prostate hyperplasia, dysuria associated with benign prostate hyperplasia, Huntington, dementia, Alzheimer and Creutzfeld-Jakob.

The present invention further provides a method for treatment and/or prophylaxis of disorders, in particular the disorders mentioned above, using an effective amount of at least one of the compounds according to the invention.

The present invention further provides a method for the treatment and/or prophylaxis of chronic renal disorders, acute and chronic renal insufficiency, diabetic, inflammatory or hypertensive nephropathies, fibrotic disorders, cardiac insufficiency, angina pectoris, hypertension, pulmonary hypertension, ischemias, vascular disorders, thromboembolic disorders, arteriosclerosis, sickle cell anemia, erectile dysfunction, benign prostate hyperplasia, dysuria associated with benign prostate hyperplasia, Huntington, dementia, Alzheimer and Creutzfeld-Jakob.

In another embodiment, the inventive compounds can also be used to treat or to prevent uterine fibroids (uterine leiomyoma or uterine myoma) in women.

Compounds of the present invention can be utilized to inhibit, block, reduce or decrease MAP4K1 activation by exogenous and/or endogenous ligands for the reduction of tumour growth and the modulation of dysregulated immune responses e.g. to block immunosuppression and increase immune cell activation and infiltration in the context of cancer and cancer immunotherapy; This method comprises administering to a mammal in need thereof, including a human, an amount of a compound of this invention, or a pharmaceutically acceptable salt, isomer, polymorph, metabolite, hydrate, solvate or ester thereof; which is effective to treat the disorder.

The present invention also provides methods of treating a variety of other disorders wherein MAP4K1 is involved such as, but not limited to, disorders with dysregulated immune responses, inflammation, vaccination for infection & cancer, viral infections, obesity and diet-induced obesity, adiposity, metabolic disorders, hepatic steatosis and uterine fibroids.

These disorders have been well characterized in humans, but also exist with a similar etiology in other mammals, and can be treated by administering pharmaceutical compositions of the present invention.

The term “treating” or “treatment” as used in the present text is used conventionally, e.g., the management or care of a subject for the purpose of combating, alleviating, reducing, relieving, improving the condition of a disease or disorder, such as liquid and solid tumours.

In accordance with a further aspect, the present invention covers compounds of general formula (I), as described supra, or stereoisomers, tautomers, N-oxides, hydrates, solvates, and salts thereof, particularly pharmaceutically acceptable salts thereof, or mixtures of same, for use in the treatment or prophylaxis of diseases, in particular cancer or conditions with dysregulated immune responses or other disorders associated with aberrant MAP4K1 signaling.

The pharmaceutical activity of the compounds according to the invention can be explained by their activity as MAP4K1 inhibitors.

In accordance with a further aspect, the present invention covers the use of compounds of general formula (I), as described supra, or stereoisomers, tautomers, N-oxides, hydrates, solvates, and salts thereof, particularly pharmaceutically acceptable salts thereof, or mixtures of same, for the treatment or prophylaxis of diseases, in particular cancer or conditions with dysregulated immune responses or other disorders associated with aberrant MAP4K1 signaling, particularly liquid and solid tumours.

In accordance with a further aspect, the present invention covers the compounds of general formula (I), as described supra, or stereoisomers, tautomers, N-oxides, hydrates, solvates, and salts thereof, particularly pharmaceutically acceptable salts thereof, or mixtures of same, for the use of treatment or prophylaxis of diseases, in particular cancer or conditions with dysregulated immune responses or other disorders associated with aberrant MAP4K1 signaling, particularly liquid and solid tumours.

In accordance with a further aspect, the present invention covers the use of compounds of general formula (I), as described supra, or stereoisomers, tautomers, N-oxides, hydrates, solvates, and salts thereof, particularly pharmaceutically acceptable salts thereof, or mixtures of same, in a method of treatment or prophylaxis of diseases, in particular cancer or conditions with dysregulated immune responses or other disorders associated with aberrant MAP4K1 signaling, particularly liquid and solid tumours.

In accordance with a further aspect, the present invention covers use of a compound of general formula (I), as described supra, or stereoisomers, tautomers, N-oxides, hydrates, solvates, and salts thereof, particularly pharmaceutically acceptable salts thereof, or mixtures of same, for the preparation of a pharmaceutical composition, preferably a medicament, for the prophylaxis or treatment of diseases, in particular cancer or conditions with dysregulated immune responses or other disorders associated with aberrant MAP4K1 signaling, particularly liquid and solid tumours.

In accordance with a further aspect, the present invention covers a method of treatment or prophylaxis of diseases, in particular cancer or conditions with dysregulated immune responses or other disorders associated with aberrant MAP4K1 signaling, particularly liquid and solid tumours, using an effective amount of a compound of general formula (I), as described supra, or stereoisomers, tautomers, N-oxides, hydrates, solvates, and salts thereof, particularly pharmaceutically acceptable salts thereof, or mixtures of same.

In accordance with a further aspect, the present invention covers pharmaceutical compositions, in particular a medicament, comprising a compound of general formula (I), as described supra, or a stereoisomer, a tautomer, an N-oxide, a hydrate, a solvate, a salt thereof, particularly a pharmaceutically acceptable salt, or a mixture of same, and one or more excipients), in particular one or more pharmaceutically acceptable excipient(s). Conventional procedures for preparing such pharmaceutical compositions in appropriate dosage forms can be utilized.

The present invention furthermore covers pharmaceutical compositions, in particular medicaments, which comprise at least one compound according to the invention, conventionally together with one or more pharmaceutically suitable excipients, and to their use for the above mentioned purposes.

It is possible for the compounds according to the invention to have systemic and/or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compounds according to the invention in crystalline and/or amorphised and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixturae agitandae), lipophilic suspensions, emulsions, ointments, creams, transdermal therapeutic systems (such as, for example, patches), milk, pastes, foams, dusting powders, implants or stents.

The compounds according to the invention can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropylcellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to a pharmaceutical composition which comprise at least one compound according to the invention, conventionally together with one or more pharmaceutically suitable excipient(s), and to their use according to the present invention. In accordance with another aspect, the present invention covers pharmaceutical combinations, in particular medicaments, comprising at least one compound of general formula (I) of the present invention and at least one or more further active ingredients, in particular for the treatment and/or prophylaxis of cancer or conditions with dysregulated immune responses or other disorders associated with aberrant MAP4K1 signaling generic name disorders, particularly liquid and solid tumours.

The term “combination” in the present invention is used as known to persons skilled in the art, it being possible for said combination to be a fixed combination, a non-fixed combination or a kit-of-parts.

A “fixed combination” in the present invention is used as known to persons skilled in the art and is defined as a combination wherein, for example, a first active ingredient, such as one or more compounds of general formula (I) of the present invention, and a further active ingredient are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

A non-fixed combination or “kit-of-parts” in the present invention is used as known to persons skilled in the art and is defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

Based upon standard laboratory techniques known to evaluate compounds useful for the treatment of cancer or conditions with dysregulated immune responses or other disorders associated with aberrant MAP4K1 signaling, by standard toxicity tests and by standard pharmacological assays for the determination of treatment of the conditions identified above in mammals, and by comparison of these results with the results of known active ingredients or medicaments that are used to treat these conditions, the effective dosage of the compounds of the present invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, it is possible for “drug holidays”, in which a patient is not dosed with a drug for a certain period of time, to be beneficial to the overall balance between pharmacological effect and tolerability. It is possible for a unit dosage to contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

### EXPERIMENTAL SECTION

NMR peak forms are stated as they appear in the spectra, possible higher order effects have not been considered. The multiplicities are stated according to the signal form which appears in the spectrum, NMR-spectroscopic effects of a higher order were not taken into consideration. Multiplicity of the NMR signals: s=singlet, d=doublet, t=triplet, q=quartet, quin=quintet, br=broad signal, m=multiplet. NMR signals: shift in [ppm]. Combinations of multiplicity could be e.g. dd=doublet from doublet.

In some cases not all H atoms are found as a signal in the NMR because the signal could overlays with a solvent signal or it is a very broad signal dependent on the NMR solvent used.

The ¹H-NMR data of selected examples/intermediates are listed in the form of ¹H-NMR peaklists. For each signal peak the δ value in ppm is given, followed by the signal intensity, reported in round brackets. The δ value-signal intensity pairs from different peaks are separated by commas. Therefore, a peaklist is described by the general form: δ₁ (intensity₁), δ₂ (intensity2), . . . , δᵢ (intensityᵢ), . . . , δₙ (intensityₙ).

The intensity of a sharp signal correlates with the height (in cm) of the signal in a printed NMR spectrum. When compared with other signals, this data can be correlated to the real ratios of the signal intensities. In the case of broad signals, more than one peak, or the center of the signal along with their relative intensity, compared to the most intense signal displayed in the spectrum, are shown. A ¹H-NMR peaklist is similar to a classical ¹H-NMR readout, and thus usually contains all the peaks listed in a classical NMR interpretation. Moreover, similar to classical ¹H-NMR printouts, peaklists can show solvent signals, signals derived from stereoisomers of title compounds (also the subject of the invention), and/or peaks of impurities. The peaks of stereoisomers, and/or peaks of impurities are typically displayed with a lower intensity compared to the peaks of the title compounds (e.g., with a purity of >90%). Such stereoisomers and/or impurities may be typical for the particular manufacturing process, and therefore their peaks may help to identify the reproduction of our manufacturing process on the basis of “by-product fingerprints”. An expert who calculates the peaks of the title compounds by known methods (MestReC, ACD simulation, or by use of empirically evaluated expectation values), can isolate the peaks of title compounds as required, optionally using additional intensity filters. Such an operation would be similar to peak-picking in classical ¹H-NMR interpretation. A detailed description of the reporting of NMR data in the form of peaklists can be found in the publication “Citation of NMR Peaklist Data within Patent Applications” (cf. Research Disclosure Database Number 605005, 2014, 1 Aug. 2014, or http://www.researchdisclosure.com/searching-disclosures). In the peak picking routine, as described in the Research Disclosure Database Number 605005, the parameter “MinimumHeight” can be adjusted between 1% and 4%. Depending on the chemical structure and/or depending on the concentration of the measured compound it may be reasonable to set the parameter “MinimumHeight”<1%.

Chemical names were generated using the ACD/Name software from ACD/Labs. In some cases generally accepted names of commercially available reagents were used in place of ACD/Name generated names.

Table 1 lists the abbreviations used in this paragraph and in the Examples section as far as they are not explained within the text body. Other abbreviations have their meanings customary per se to the skilled person.

The various aspects of the invention described in this application are illustrated by the following examples which are not meant to limit the invention in any way.

The example testing experiments described herein serve to illustrate the present invention and the invention is not limited to the examples given.

### Experimental Section—General Part

All reagents, for which the synthesis is not described in the experimental part, are either commercially available, or are known compounds or may be formed from known compounds by known methods by a person skilled in the art.

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using for example prepacked silica gel cartridges, e.g. Biotage SNAP cartidges KP-Sil® or KP-NH® in combination with a Biotage autopurifier system (SP4® or Isolera Four®) and eluents such as gradients of hexane/ethyl acetate, DCM/methanol, or DCM/ethanol. In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and acetonitrile which may contain additives such as trifluoroacetic acid, formic acid or aqueous ammonia.

In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art, or be used as salts in subsequent biological assays. It is to be understood that the specific form (e.g. salt, free base etc.) of a compound of the present invention as isolated and as described herein is not necessarily the only form in which said compound can be applied to a biological assay in order to quantify the specific biological activity.

### Experimental Section—General Synthesis

The following paragraphs outline a variety of synthetic approaches suitable to prepare compounds of the general formula (I), and intermediates useful for their synthesis.

In addition to the routes described below, also other routes may be used to synthesize the title compounds, in accordance with common general knowledge of a person skilled in the art of organic synthesis. The order of transformations exemplified in the following schemes is therefore not intended to be limiting, and suitable synthesis steps from various schemes can be combined to form additional synthesis sequences. In addition, interconversion of any of the substituents, in particular R¹, R² and R⁴, which are as defined in formula (I) supra, can be achieved before and/or after the exemplified transformations. These modifications can be, for example, the introduction of protective groups, cleavage of protective groups, reduction or oxidation of functional groups, halogenation, metallation, metal catalysed coupling reactions, exemplified by but not limited to e.g. Buchwald, Suzuki, Sonogashira and Ullmann coupling, ester saponifications, amide coupling reactions, and/or substitution or other reactions known to a person skilled in the art. These transformations include those which introduce a functionality allowing for further interconversion of substituents. Appropriate protective groups and their introduction and cleavage are well-known to a person skilled in the art (see for example T. W. Greene and P. G. M. Wuts in Protective Groups in Organic Synthesis, 4^{th} edition, Wiley 2006).

Further, it is possible that two or more successive steps may be performed without work-up being performed between said steps, e.g. a “one-pot” reaction, as it is well-known to a person skilled in the art.

The syntheses of the derivatives according to the present invention are preferably carried out according to the general synthetic sequence, shown in schemes 1-3.

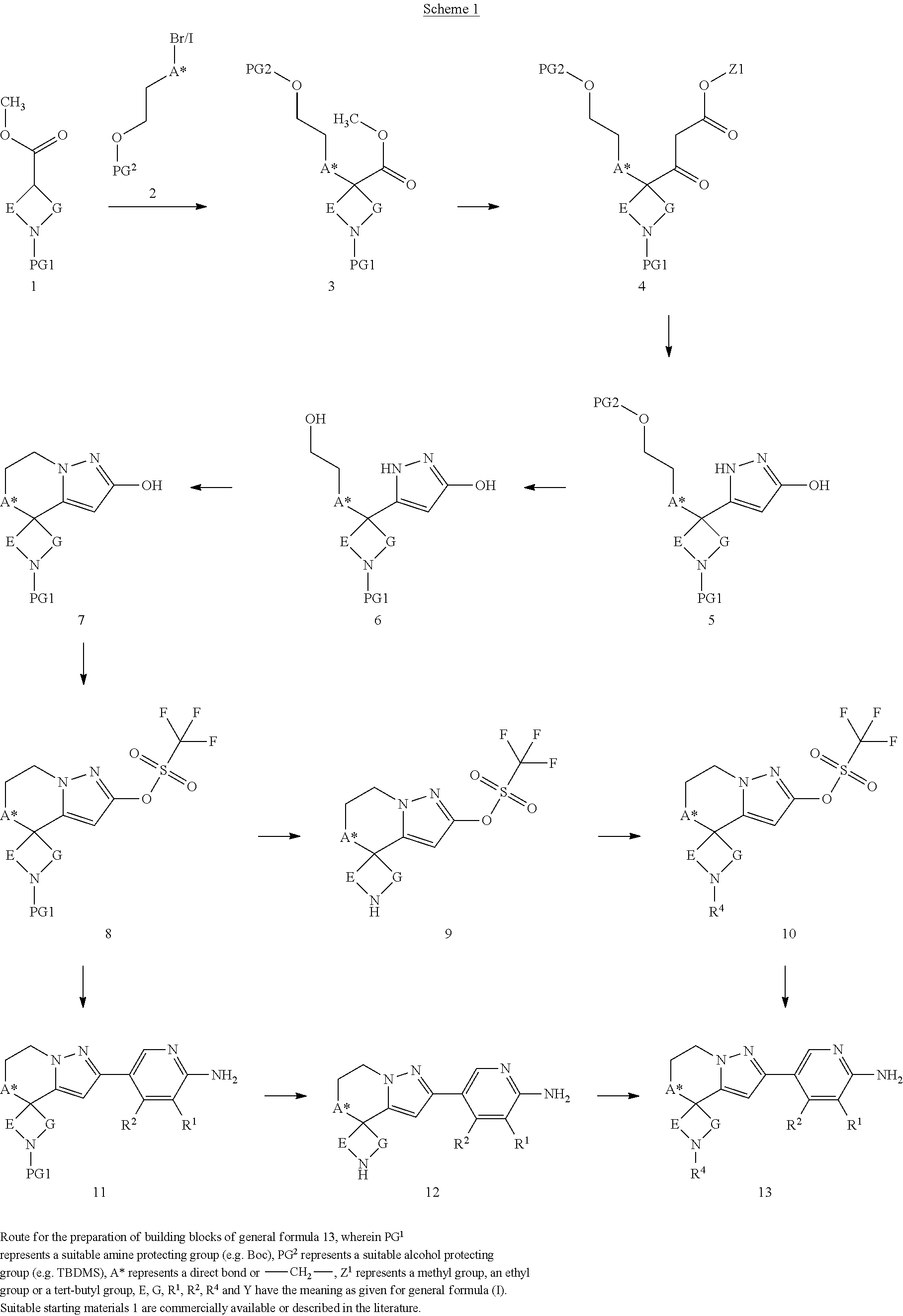

### Step 1→3 (Scheme 1)

Alkylation

In the first step (scheme 1), ester derivative 1 can be alkylated using an alkylbromide or alkyliodide of formula 2 to give the desired product 3.

For example ester 1 can be alkylated using (2-bromoethoxy)(tert-butyl)dimethylsilane 2 in an organic solvent such as THF in the presence of a base such as LiHMDS or LDA.

### Step 3→4 (Scheme 1)

Beta-Keto Ester Formation

Methylester 3 is reacted with a methyl acetate or tert-butyl acetate to give beta-keto esters of the general formula 4. Typically the reaction is performed in the presence of a base like LiHMDS or LDA in an organic solvent like THF at a temperature range between −78° C. and room temperature.

### Step 4→5 (Scheme 1)

Pyrazol Formation

beta-Keto esters of formula 4 can be converted with hydrazine to the corresponding pyrazole derivatives of formula 5. Typically the reaction is performed in an organic solvent like ethanol at a temperature between −20° C. and the boiling point of the selected solvent.

### Step 5→6 (Scheme 1)

Deprotection of PG²

The protecting group PG² of pyrazoles of formula 5 can be cleaved to give an alcohol of formula 6. The cleavage of suitable alcohol protecting groups is well-known to the person skilled in the art (see for example P. G. M. Wuts and T. W. Greene in “Protective Groups in Organic Synthesis”, 4^{th} edition, Wiley 2006). For example, when PG2 in compounds of formula 5 is TBDMS, cleavage can be achieved using e.g. HCl in an organic solvent such as methanol or TBAF in an organic solvent such as THF.

### Step 6→7 (Scheme 1)

Ring Closure

Alcohols of formula 6 can be converted to spiro compounds of formula 7 by ring closing reactions. For example, alcohols of formula 6 can be reacted with mesylchloride and DIEA in an organic solvent like DCM to give the corresponding mesylate, which is then reacted to give spiro compounds of formula 7, e.g. in the presence of a base like NaOH using a solvent mixture like methanol/water. Moreover, ring closure can be achieved using Mitsunobu conditions known to the skilled person. For example, DEAD (diethyl azodicarboxylate) or DIAD (diisopropyl azodicarboxylate), triphenylphosphine in an organic solvent such as for example THF can be used.

### Step 7→8 (Scheme 1)

Triflate Formation

Spiro compounds of formula 7 can be converted to triflates of formula 8. Typically the reaction is performed using Tf₂O in the presence of a base like DIEA in an organic solvent like DCM at a temperature range between −78° C. and room temperature. Alternatively the reaction is performed using N,N-bis-(trifluormethansulfonyl)-aniline in the presence of a base like DIEA in an organic solvent like THF at a temperature range between room temperature and the boiling point of the selected solvent.

### Step 8→9 (Scheme 1)

Deprotection of PG¹

The protecting group PG¹ of spiro compounds of formula 8 can be cleaved to give amines of formula 9. The cleavage of suitable amine protecting groups is well-known to the person skilled in the art (see for example P. G. M. Wuts and T. W. Greene in “Protective Groups in Organic Synthesis”, 4^{th} edition, Wiley 2006). For example, when PG¹ in compounds of formula 8 is BOC, cleavage can be achieved using e.g. TFA in an organic solvent such as DCM.

### Step 9→10 (Scheme 1)

Amine Decoration

Amines of formula 9 can be functionalized with a broad variety of substituents to give compounds of formula 10. For examples, secondary amines of formula 9 can be reacted to give for example tertiary amines, amides, ureas, carbamates or sulphonamides of formula 10. All these transformations are known to the skilled person.

### Step 10→13 (Scheme 1)

C—C Cross Coupling Reaction

Compounds of general formula 10 can be reacted with a boronic acid derivative

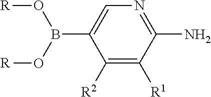

to give a compound of formula 13. The boronic acid derivative may be a boronic acid (R=—H) or an ester of the boronic acid, e.g. its isopropyl ester (R=—CH(CH₃)₂), preferably an ester derived from pinacol in which the boronic acid intermediate forms a 4,4,5,5-tetramethyl-1,3,2-dioxaborolane (R—R=—C(CH₃)₂—C(CH₃)₂—). The coupling reaction is catalyzed by palladium catalysts, e.g. by Pd(0) catalysts like tetrakis(triphenylphosphine)palladium(0) [Pd(PPh₃)₄], tris(dibenzylideneacetone)di-palladium(0) [Pd₂(dba)₃], or by Pd(II) catalysts like dichlorobis(triphenylphosphine)-palladium (II) [Pd(PPh₃)₂Cl₂], palladium (II) acetate and triphenylphosphine, [1,1′-bis(diphenylphosphino)ferrocene] palladium dichloride or by second generation XPhos Pd (Chloro(2-dicyclohexylphosphino-2′,4′,6′-triisopropyl-1,1′-biphenyl)[2-(2′-amino-1,1′-biphenyl)]palladium(II), X-Phos aminobiphenyl palladium chloride precatalyst). The reaction is preferably carried out in a mixture of a solvent like 1,2-dimethoxyethane, dioxane, DMF, DME, THF, or isopropanol with water and in the presence of a base like potassium carbonate, sodium bicarbonate or potassium phosphate. (review: D. G. Hall, Boronic Acids, 2005 WILEY-VCH Verlag GmbH & Co. KGaA, Weinheim, ISBN 3-527-30991-8 and references cited therein). The reaction is performed at temperatures ranging from room temperature to the boiling point of the solvent. Further on, the reaction can be performed at temperatures above the boiling point under pressure. The reaction is preferably completed after 1 to 36 hours.

### Step 8→11 (Scheme 1)

C—C Cross Coupling Reaction

Compounds of general formula 8 can be reacted with a boronic acid derivative

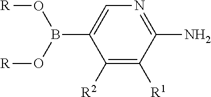

to give a compound of formula 11. The boronic acid derivative may be a boronic acid (R=—H) or an ester of the boronic acid, e.g. its isopropyl ester (R=—CH(CH₃)₂), preferably an ester derived from pinacol in which the boronic acid intermediate forms a 4,4,5,5-tetramethyl-1,3,2-dioxaborolane (R—R=—C(CH₃)₂—C(CH₃)₂—). The coupling reaction is catalyzed by palladium catalysts, e.g. by Pd(0) catalysts like tetrakis(triphenylphosphine)palladium(0) [Pd(PPh₃)₄], tris(dibenzylideneacetone)di-palladium(0) [Pd₂(dba)₃], or by Pd(II) catalysts like dichlorobis(triphenylphosphine)-palladium (II) [Pd(PPh₃)₂Cl₂], palladium (II) acetate and triphenylphosphine, [1,1′-bis(diphenylphosphino)ferrocene]palladium dichloride or by second generation XPhos Pd (Chloro(2-dicyclohexylphosphino-2′,4′,6′-triisopropyl-1,1′-biphenyl)[2-(2′-amino-1,1′-biphenyl)]palladium(II), X-Phos aminobiphenyl palladium chloride precatalyst). The reaction is preferably carried out in a mixture of a solvent like 1,2-dimethoxyethane, dioxane, DMF, DME, THF, or isopropanol with water and in the presence of a base like potassium carbonate, sodium bicarbonate or potassium phosphate. (review: D. G. Hall, Boronic Acids, 2005 WILEY-VCH Verlag GmbH & Co. KGaA, Weinheim, ISBN 3-527-30991-8 and references cited therein). The reaction is performed at temperatures ranging from room temperature to the boiling point of the solvent. Further on, the reaction can be performed at temperatures above the boiling point under pressure. The reaction is preferably completed after 1 to 36 hours.

### Step 11→12 (Scheme 1)

Deprotection of PG¹

The protecting group PG¹ of spiro compounds of formula 11 can be cleaved to give amines of formula 12. The cleavage of suitable amine protecting groups is well-known to the person skilled in the art (see for example P. G. M. Wuts and T. W. Greene in “Protective Groups in Organic Synthesis”, 4^{th} edition, Wiley 2006). For example, when PG¹ in compounds of formula 12 is BOC, cleavage can be achieved using e.g. TFA in an organic solvent such as DCM.

### Step 12→13 (Scheme 1)

Amine Decoration

Amines of formula 12 can be functionalized with a broad variety of substituents to give compounds of formula 13. For examples, secondary amines of formula 13 can be reacted to give tertiary amines, amides, ureas, carbamates or sulphonamides of formula 13. All these transformations are known to the skilled person.

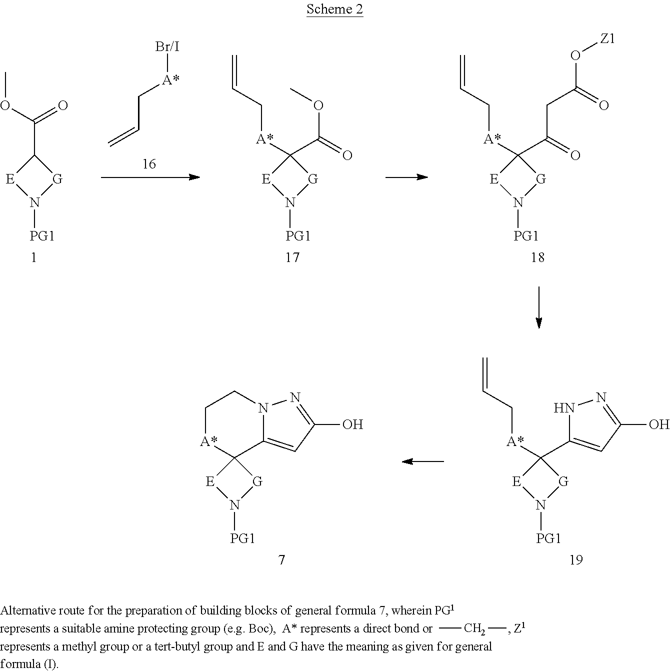

### Step 1→17 (Scheme 2)

Alkylation

Compounds of the general formula 1 can be converted to compounds of the general formula 17 by alkylation. Typically the reaction is performed with an alkylating agent such as for example 16, a base such as LiHMDS or LDA in an organic solvent such as THF.

### Step 17→18 (Scheme 2)

Beta-Keto Ester Formation

Methylester 17 is reacted with a methyl acetate or tert-butyl acetate to give beta-keto esters of the general formula 18. Typically the reaction is performed in the presence of a base like LiHMDS or LDA in an organic solvent like THF at a temperature range between −78° C. and room temperature.

### Step 18→19 (Scheme 2)

Pyrazol Formation

beta-Keto esters of formula 18 can be converted with hydrazine to the corresponding pyrazole derivatives of formula 19. Typically the reaction is performed in an organic solvent like ethanol at a temperature between −20° C. and the boiling point of the selected solvent.

### Step 19→7 (Scheme 2)

Cyclization

Compounds of the general formula 19 can be converted to compounds of formula 7. Reaction conditions are known to the skilled person. Typically the olefin part is converted to a diol followed by cleavage of the diol forming two aldehydes. The so called Lemieux-Johnson reaction is performed with osmium tetroxide and sodium metaperiodate in a mixture of water and an organic solvent for example dioxane at 5-40° C., and preferably at room temperature. The osmium tetroxide can be generated in situ from an osmium(VI) salt (e.g. K₂OsO₄ dihydrate) easily oxidizable to osmium(VIII) by sodium metaperiodate. In another preferred procedure using the Jin-protocol 2,6-lutidine was added to the reaction mixture.

The aldehyde is reduced to the alcohol with e.g. sodium boronhydride in an organic solvent, for example an alcohol (e.g. methanol) at temperature starting from −10° C. to 10° C. (preferably at 0° C.) and finally increasing to elevated temperature e.g. room temperature to 50° C.

Then, the alcohols can be converted to spiro compounds of formula 7 by ring closing reactions. For example, the alcohols can be reacted with mesylchloride and DIEA in an organic solvent like DCM to give the corresponding mesylate, which is then reacted to give spiro compounds of formula 7, e.g. in the presence of a base like NaOH using a solvent mixture like methanol/water. Moreover, ring closure can be achieved using Mitsunobu conditions known to the skilled person. For example, DEAD (diethyl azodicarboxylate) or DIAD (diisopropyl azodicarboxylate), triphenylphosphine in an organic solvent such as for example THF can be used.

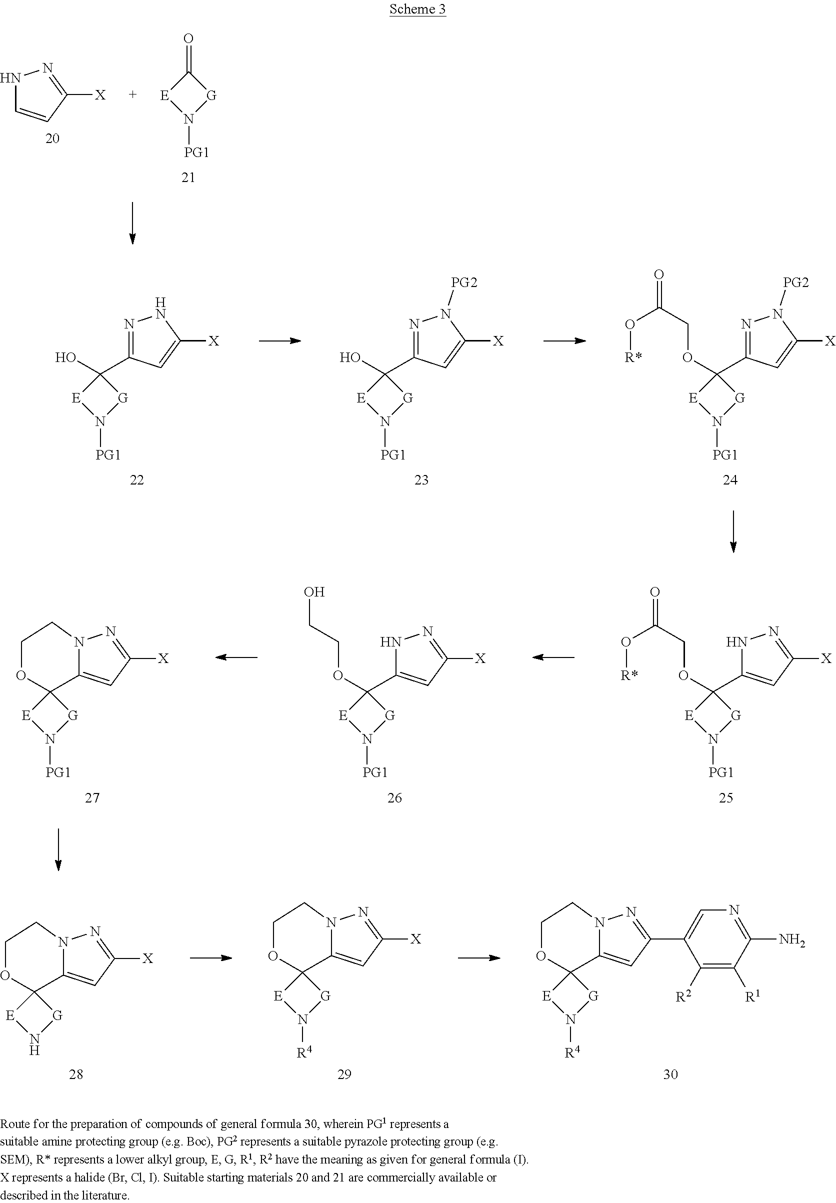

### Step 20+21→22 (Scheme 3)

Pyrazole Addition to the Carbonyl Group

Ketones of the general formula 21 and pyrazoles of the general formula 20 can be converted to compounds of the general formula 22. The conversion is known to the person skilled in the art. For example, the conversion can be carried out in analogy to a literature procedure described in Tetrahedron, 1983, 39, 2023-2029.

### Step 22→23 (Scheme 3)

Protection with PG²

Compounds of the general formula 22 can be converted to compounds of the general formula 23 using a suitable protecting group to protect the pyrazole NH. Protecting groups for pyrazoles are known to the skilled person (see for example P. G. M. Wuts and T. W. Greene in “Protective Groups in Organic Synthesis”, 4^{th} edition, Wiley 2006). For example, when PG2 in compounds of formula 23 is SEM, then 2-(trimethylsilyl)ethoxymethylchloride, a base such as sodium hydride in an organic solvent such as THF can be used.

### Step 23→24 (Scheme 3)

Alkylation of the Alcohol

Alcohols of the general formula 23 can be converted to compounds of the general formula 24 in an alkylation reaction known to the skilled person. For example, bromo ethyl acetate, a base, such as sodium hydride in an organic solvent such as dioxane at elevated temperature can be used.

### Step 24→25 (Scheme 3)

Deprotection of PG²

The protecting group PG² of pyrazole compounds of general formula 24 can be cleaved to give compounds of formula 25. The cleavage of suitable pyrazol protecting groups is well-known to the person skilled in the art (see for example P. G. M. Wuts and T. W. Greene in “Protective Groups in Organic Synthesis”, 4^{th} edition, Wiley 2006). For example, when PG2 in compounds of formula 24 is SEM, cleavage can be achieved using e.g. TFA or TBAF in an organic solvent such as DCM.

### Step 25→26 (Scheme 3)

Reduction of the Ester

Esters of the general formula 25 (R*=lower alkyl group) can be converted to the corresponding alcohols of the general formula 26 with hydride reducing agents known to the skilled person. For example lithium borohydride in an organic solvent, such as THF can be used.

### Step 26→27 (Scheme 3)

Mitsunobu Reaction

Compounds of the general formula 26 can be converted to the corresponding morpholine derivatives of the general formula 27 using Mitsunobu conditions known to the skilled person. For example, DEAD (diethyl azodicarboxylate) or DIAD (diisopropyl azodicarboxylate), triphenylphosphine in an organic solvent such as for example THF can be used.

### Step 27→28 (Scheme 3)

Deprotection of PG¹

The protecting group PG¹ of spiro compounds of formula 27 can be cleaved to give amines of formula 28. The cleavage of suitable amine protecting groups is well-known to the person skilled in the art (see for example P. G. M. Wuts and T. W. Greene in “Protective Groups in Organic Synthesis”, 4^{th} edition, Wiley 2006). For example, when PG2 in compounds of formula 27 is BOC, cleavage can be achieved using e.g. TFA in an organic solvent such as DCM.

### Step 28→29 (Scheme 3)

Amine Decoration

Amines of formula 28 can be functionalized with a broad variety of substituents to give compounds of formula 29. For examples, secondary amines of formula 29 can be reacted to give tertiary amines, amides, ureas, carbamates or sulphonamides of formula 29. All these transformations are known to the skilled person.

### Step 29→30 (Scheme 3)

C—C Cross Coupling Reaction

Halogen compounds of general formula 29 can be reacted with a boronic acid derivative

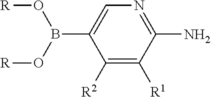

to give a compound of formula 30. The boronic acid derivative may be a boronic acid (R=—H) or an ester of the boronic acid, e.g. its isopropyl ester (R=—CH(CH₃)₂), preferably an ester derived from pinacol in which the boronic acid intermediate forms a 4,4,5,5-tetramethyl-1,3,2-dioxaborolane (R—R=—C(CH₃)₂—C(CH₃)₂—). The coupling reaction is catalyzed by palladium catalysts, e.g. by Pd(0) catalysts like tetrakis(triphenylphosphine)palladium(0) [Pd(PPh₃)₄], tris(dibenzylideneacetone)di-palladium(0) [Pd₂(dba)₃], or by Pd(II) catalysts like dichlorobis(triphenylphosphine)-palladium (II) [Pd(PPh₃)₂Cl₂], palladium (II) acetate and triphenylphosphine, [1,1′-bis(diphenylphosphino)ferrocene] palladium dichloride or by second generation XPhos Pd (Chloro(2-dicyclohexylphosphino-2′,4′,6′-triisopropyl-1,1′-biphenyl)[2-(2′-amino-1,1′-biphenyl)]palladium(II), X-Phos aminobiphenyl palladium chloride precatalyst). The reaction is preferably carried out in a mixture of a solvent like 1,2-dimethoxyethane, dioxane, DMF, DME, THF, or isopropanol with water and in the presence of a base like potassium carbonate, sodium bicarbonate or potassium phosphate. (review: D. G. Hall, Boronic Acids, 2005 WILEY-VCH Verlag GmbH & Co. KGaA, Weinheim, ISBN 3-527-30991-8 and references cited therein). The reaction is performed at temperatures ranging from room temperature to the boiling point of the solvent. Further on, the reaction can be performed at temperatures above the boiling point under pressure. The reaction is preferably completed after 1 to 36 hours.

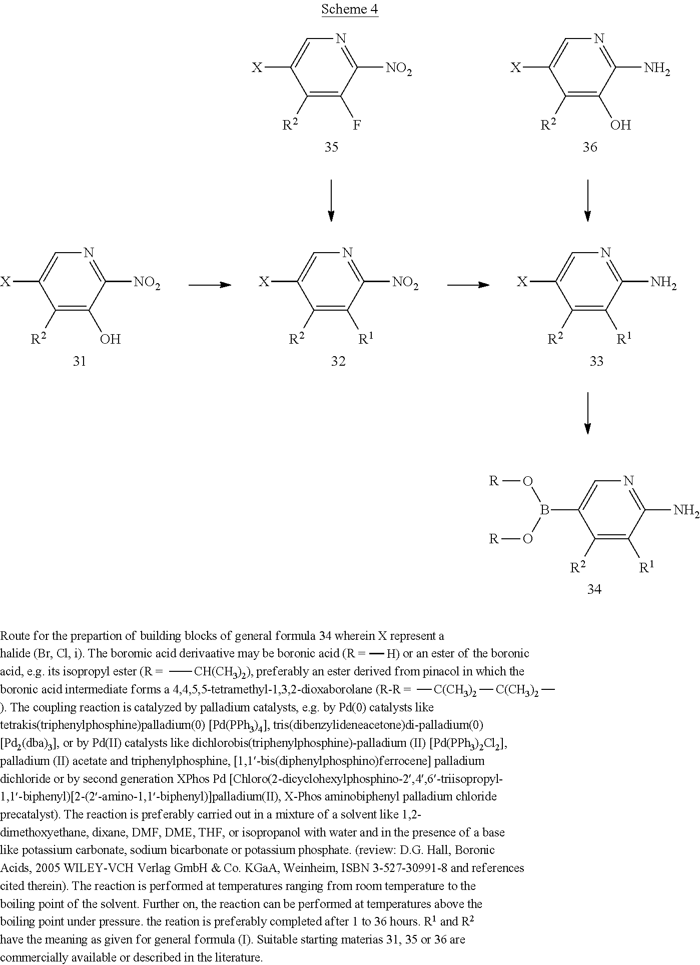

### Step 31→32 (Scheme 4)

Hydroxy Group Transformation

Nitropyridine 31 can be converted to nitropyridine 32 using Mitsunobu conditions known to the skilled person. For example, DEAD (diethyl azodicarboxylate) or DIAD (diisopropyl azodicarboxylate), triphenylphosphine or tributylphosphine in an organic solvent such as for example THF, dioxane, DCM, acetonitrile or toluene can be used.

Alternatively, pyridine 31 can be alkylated using alkyl halides or sulfonates (tosylates, mesylates, triflates or nonaflates) and a base such as NaH, Cs₂CO3, K₂CO₃, NaOH in organic solvents such as THF or DMF.

### Step 35→32 (Scheme 4)

Nucleophilic Aromatic Substitution

Fluoropyridine 35 can be converted to nitropyridine 32 using a nucleophilic aromatic substitution. For example, by using a nucleophile in the presence of a base such as NaH, Cs2CO3, K2CO3, NaOH in an organic solvent like THF, dioxane, DMA or acetonitrile.

### Step 32→33 (Scheme 4)

Reduction

Pyridine 32 can be converted to aminopyridine 33 by reduction of the nitro group. For example, by using a metal such as Fe or Zn and acetic acid or hydrochloric acid or ammonium chloride. Alternatively, this reduction can be performed with hydrogen using a catalyst such as Pt/C, Pd/C or Raney Nickel.

### Step 36→33 (Scheme 4)

Hydroxy Group Alkylation

Aminopyridine 36 can be converted to aminopyridine 33 using Mitsunobu conditions known to the skilled person. For example, DEAD (diethyl azodicarboxylate) or DIAD (diisopropyl azodicarboxylate), triphenylphosphine in an organic solvent such as for example THF can be used.

Alternatively, aminopyridine 36 can be alkylated using alkyl halides or alkylmesylates and a base such as sodium hydride, cesium carbonate, potassium carbonate or sodium hydroxide in organic solvents such as THF, DMF or ethanol.

### Step 33→34 (Scheme 4)

Borylation

Pyridine halide 33 can be converted to the corresponding boronic acid or ester 34 using Miyaura borylation conditions know from the person skilled in the art. For example, using bis-pinacol borane, a palladium catalyst such as Pd(dppf)Cl2, and a base for example potassium acetate in organic solvent such as dioxane or DMSO.

The steps for the synthesis sequence giving rise to spiro compounds of formula 7, 15 or 30 may be also interchanged using similar reaction conditions for each step as described above.

### Abbreviations

TABLE 1
Abbreviations

| Abbreviation | Meaning |
|---|---|
| CDI | carbonyldiimidazole |
| DCM | dichloromethane |
| DIAD | diisopropyl azodicarboxylate |
| DMF | N,N-dimethylformamide |
| DMSO | dimethylsulfoxide |
| h | hour(s) |
| HATU | (1-[bis(dimethylamino)methylene]-1H-1,2,3-triazolo[4,5- |
| | b]pyridinium 3-oxide hexafluorophosphate |
| LDA | Lithium diisopropylamide |
| LiHMDS | Lithium bis(trimethylsilyl)amide |
| MeOH | methanol |
| min | minute(s) |
| MTBE | methyl tert-butyl ether |
| NaOH | sodium hydroxide |
| Pd(dppf)Cl₂ | [1,1′-bis(diphenylphosphino)ferrocene]dichloropalladium(II) |
| rt | room temperature |
| Rₜ | retention time |
| TBAF | tetrabutylammonium fluoride |
| TFA | trifluoro acetic acid |
| THF | tetrahydrofuran |
| TLC | thin layer chromatography |
| XPhos Pd G2 | (2′-aminobiphenyl-2-yl)(chloro)palladium-dicyclohexyl |
| | [2′,4′,6′-tri(propan-2-yl)biphenyl-3-yl]phosphane (1:1) |

### Experimental Section—Methods

Analytical LC-MS methods:

Method 1:

Instrument: Waters Acquity UPLCMS SingleQuad; Column: Acquity UPLC BEH C18 1.7 μm, 50×2.1 mm; eluent A: water+0.2 vol % aq. ammonia (32%), eluent B: acetonitrile; gradient: 0-1.6 min 1-99% B, 1.6-2.0 min 99% B; flow 0.8 mL/min; temperature: 60° C.; DAD scan: 210-400 nm.

Method 2:

Instrument: Waters Acquity UPLCMS SingleQuad; Column: Acquity UPLC BEH C18 1.7 μm, 50×2.1 mm; eluent A: water+0.1 vol % formic acid (99%), eluent B: acetonitrile; gradient: 0-1.6 min 1-99% B, 1.6-2.0 min 99% B; flow 0.8 mL/min; temperature: 60° C.; DAD scan: 210-400 nm. It would be similar in the retention time

Method 5:

Instrument: Thermo Scientific FT-MS UHPLC+: Thermo Scientific UltiMate 3000; column: Waters, HSST3, 2.1×75 mm, C18 1.8 μm; eluent A: water+0.01% formic acid; eluent B: acetonitrile+0.01% formic acid; gradient: 0.0 min 10% B→2.5 min 95% B→3.5 min 95% B; temperature: 50° C.; flow: 0.90 mL/min; UV-detection: 210 nm/optimum integration path 210-300 nm.

Method 6:

Instrument: Waters Acquity UPLCMS SingleQuad; Column: Acquity UPLC BEH C18 1.7 μm, 50×2.1 mm; eluent A: water+0.1 vol % formic acid (99%), eluent B: acetonitrile; gradient: 0-1.7 min 1-45% B, 1.7-1.72 min 45-99% B, 1.72-2.0 min 99% B; flow 0.8 mL/min; temperature: 60° C.; DAD scan: 210-400 nm.

Method 7:

Instrument: Waters Acquity UPLCMS SingleQuad; Column: Acquity UPLC BEH C18 1.7μ, 50×2.1 mm; eluent A: water+0.1 vol % formic acid; eluent B: acetonitrile; gradient: 0-1.6 min 1-99% B, 1.6-2.0 min 99% B; flow: 0.8 mL/min; temperature: 60° C.; DAD scan: 210-400 nm.

Method 8:

Instrument: Waters Acquity UPLCMS SingleQuad; Column: Acquity UPLC BEH C18 1.7 μm, 50×2.1 mm; Eluent A: water+0.2 vol-% ammonia (32%), Eluent B: acetonitrile; gradient: 0-1.6 min 1-99% B, 2.6-3.0 min 99% B; flow: 0.8 mL/min; temperature: 60° C.; DAD scan: 210-400 nm.

### Intermediate 1

### 5-(4,4,5,5-tetramethyl-1,3,2-dioxaborolan-2-yl)-3-(trifluoromethyl)pyridin-2-amine

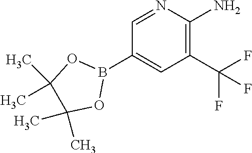 [SMILES: CC1(C)OB(c2cnc(N)c(C(F)(F)F)c2)OC1(C)C]

5-Bromo-3-(trifluoromethyl)pyridin-2-amine (2.00 g, 8.30 mmol) and 4,4,4′,4′,5,5,5′,5′-octamethyl-2,2′-bi-1,3,2-dioxaborolane (2.53 g, 9.96 mmol) were solubilised in dioxane (43 mL), potassium acetate (2.44 g, 24.9 mmol) and Pd(dppf)Cl₂ (607 mg, 830 μmol) were added and the mixture was stirred overnight at 100° C. The mixture was diluted with dichloromethane, filtered and evaporated. The residue was purified by flash chromatography to give 2.70 g of the target compound.

1H-NMR (400 MHz, DMSO-d6) δ[ppm]: 1.052 (0.88), 1.065 (16.00), 1.069 (1.16), 1.145 (0.90), 1.154 (8.40), 1.163 (1.61), 1.176 (0.70), 1.269 (11.09), 1.290 (1.05), 3.332 (0.74), 3.936 (2.59), 7.799 (0.47), 7.803 (0.48), 7.939 (0.72), 8.375 (0.46), 8.377 (0.46).

### Intermediate 2

### 2-amino-5-(4,4,5,5-tetramethyl-1,3,2-dioxaborolan-2-yl)pyridine-3-carbonitrile

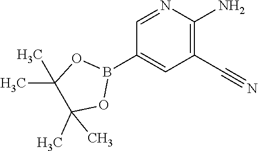 [SMILES: CC1(C)OB(c2cnc(N)c(C#N)c2)OC1(C)C]

2-Amino-5-bromopyridine-3-carbonitrile (1.40 g, 7.07 mmol), 4,4,4′,4′,5,5,5′,5′-octamethyl-2,2′-bi-1,3,2-dioxaborolane (1.97 g, 7.78 mmol), Pd(dppf)Cl₂ (289 mg, 353 μmol) and potassium acetate (2.08 g, 21.2 mmol) were stirred in degassed dioxane (35 mL) at 100° C. overnight. The reaction mixture was filtered and used without further work up or purification in the next step.

¹H NMR (400 MHz, DMSO-d₆) δ ppm 1.27 (s, 12H) 7.33 (s, 2H) 7.92 (d, 1H) 8.37 (d, 1H).

### Intermediate 3

### ethyl 2-(3-chloropyridin-4-yl)-2-methylpropanoate

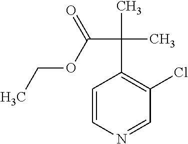

3-Chloropyridine-4-carbonitrile (500 mg, 3.61 mmol) in THF (12 mL) was cooled to −78° C. under nitrogen atmosphere then ethyl 2-methylpropanoate (419 mg, 3.61 mmol) was added and potassium bis(trimethylsilyl)amide (7.9 mL, 0.50 M in toluene, 4.0 mmol) was added dropwise, the mixture was stirred 10 minutes before removing the cooling bath and stirred 1 h at rt. The reaction was quenched with saturated ammonium chloride solution and extracted three times with dichloromethane. The combined organic phases were dried over sodium sulfate and concentrated. The residue was purified by flash chromatography to afford 300 mg (36% yield) of the title compound.

### Intermediate 4

### 2-(3-chloropyridin-4-yl)-2-methylpropanoic acid

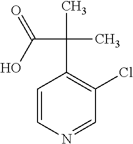

To a stirred solution of ethyl 2-(3-chloropyridin-4-yl)-2-methylpropanoate (300 mg, 1.32 mmol) in methanol (8.5 mL) and THF (26 mL) was added lithium hydroxide (5.3 mL, 1.0 M, 5.3 mmol) and stirred overnight at rt. Lithium hydroxide (2.8 mL, 1.0 M, 2.8 mmol) was added to the mixture and stirred overnight at rt, then the mixture was heated for 4 h at 40° C. and then overnight at 50° C. Potassium hydroxide (1.3 mL, 1.0 M, 1.3 mmol) was added and stirred 4 h at 50° C. and then 72 h at rt. Potassium hydroxide (1.3 mL, 1.0 M, 1.3 mmol) was added again and stirred 2 days at 50° C. The reaction mixture was divided in two portions and stirred in the microwave at 100° C. for 1 h. The reaction mixture was diluted with water, evaporated from organic solvents and extracted three times with dichloromethane. The aqueous phase was acidified to pH 2-3 using hydrochloric acid (9.5 mL, 1M) and extracted with dichloromethane (+10% ethanol). The combined organic phases were concentrated to give 170 mg (64% yield) of the title compound.

### Intermediate 5

### 3-chloro-4-(2-isocyanatopropan-2-yl)pyridine

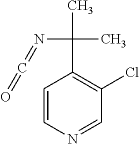

To a stirred solution of 2-(3-chloropyridin-4-yl)-2-methylpropanoic acid (170 mg, 852 μmol) in dioxane (2.8 mL) and triethylamine (150 μL) was added diphenyl phosphorazidate (240 μL, 1.1 mmol) and stirred at rt overnight. The reaction mixture was used in the next step without purification.

### Intermediate 6

### (rac)-1-tert-butyl-3-methyl-3-(2-{[tert-butyl(dimethyl)silyl]oxy}ethyl)pyrrolidine-1,3-dicarboxylate

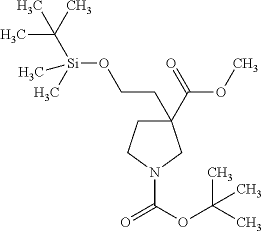 [SMILES: COC(=O)C1(CCO[Si](C)(C)C(C)(C)C)CCN(C(=O)OC(C)(C)C)C1]

1-Tert-butyl 3-methyl pyrrolidine-1,3-dicarboxylate (9.59 g, 41.83 mmol) was dissolved in THF (96 mL) under argon atmosphere. It was cooled down to −78° C. and LiHMDS (83.7 mL, 83.7 mmol, 1 M in THF) was added. It was stirred 1 h at −78° C. and then (2-bromoethoxy)(tert-butyl)dimethylsilane (20.01 g, 83.7 mmol) in THF (53 mL) were added dropwise. It was stirred overnight and allowed to reach rt. On an ice bath saturated ammonium chloride solution (160 mL) was added and stirred for 1 h. It was extracted with ethyl acetate/THF, dried through a silicone filter and concentrated. The residue was purified by flash chromatography yielding 9.19 g (57%) of the title compound.

¹H NMR (400 MHz, DMSO-d₆) δ ppm: 0.01 (s, 6H), 0.85 (s, 9H), 1.38 (s, 9H), 1.75-1.97 (m, 3H), 2.14-2.25 (m, 1H), 3.02-3.17 (m, 2H), 3.24-3.30 (m, 1H), 3.46-3.64 (m, 5H), 3.67-3.82 (m, 1H).

### Intermediate 7

### (rac)-tert-butyl-3-(3-tert-butoxy-3-oxopropanoyl)-3-(2-{[tert-butyl(dimethyl)silyl]oxy}ethyl)pyrrolidine-1-carboxylate

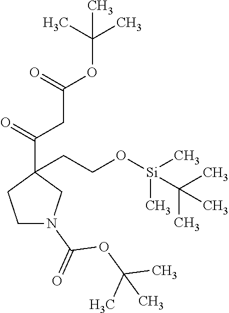 [SMILES: CC(C)(C)OC(=O)CC(=O)C1(CCO[Si](C)(C)C(C)(C)C)CCN(C(=O)OC(C)(C)C)C1]

LiHMDS (6.5 mL, 1.0 M in THF, 6.5 mmol) was diluted in THF (10 mL) under argon and cooled down to −78° C. then tert-butyl acetate (870 μL, 6.5 mmol) was added dropwise and stirred for 1 h at −78° C. Then (rac)-1-tert-butyl-3-methyl-3-(2-{[tert-butyl(dimethyl)silyl]oxy}ethyl)pyrrolidine-1,3-dicarboxylate (500 mg, 1.29 mmol) dissolved in THF (10 mL) was added dropwise. The reaction mixture was allowed to warm up to rt and stirred overnight at rt. The mixture was poured into saturated ammonium chloride solution and extracted twice with ethyl acetate, dried over sodium sulfate and concentrated to afford 500 mg (82% yield) of the title compound.

¹H-NMR (400 MHz, DMSO-d6) δ[ppm]: −0.029 (0.99), −0.011 (0.72), −0.003 (16.00), 0.009 (14.92), 0.017 (0.48), 0.057 (1.56), 0.069 (1.62), 0.079 (0.51), 0.168 (0.89), 0.840 (7.12), 1.345 (0.44), 1.381 (10.57), 1.443 (0.60), 1.987 (0.53), 2.518 (0.98), 2.522 (0.66), 3.593 (0.43), 3.655 (0.76).

### Intermediate 8

### (rac)-tert-butyl-3-(2-{[tert-butyl(dimethyl)silyl]oxy}ethyl)-3-(5-hydroxy-1H-pyrazol-3-yl)pyrrolidine-1-carboxylate

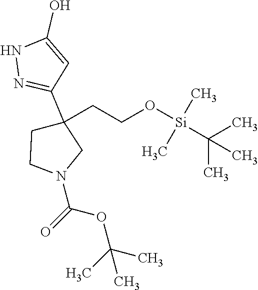 [SMILES: CC(C)(C)OC(=O)N1CCC(CCO[Si](C)(C)C(C)(C)C)(c2cc(O)[nH]n2)C1]

(Rac)-tert-butyl-3-(3-tert-butoxy-3-oxopropanoyl)-3-(2-{[tert-butyl(dimethyl)silyl]oxy}ethyl)pyrrolidine-1-carboxylate (8.60 g, 18.2 mmol) and hydrazine hydrate (3.5 mL, 73 mmol) were dissolved in ethanol (180 mL) under argon and stirred for 6 h at 80° C. The reaction mixture was concentrated to give 10.5 g of the title compound.

¹H-NMR (400 MHz, DMSO-d6) δ[ppm]: −0.185 (0.47), −0.149 (0.52), −0.035 (13.08), −0.030 (1.92), 0.685 (0.59), 0.831 (11.97), 0.843 (16.00), 1.039 (6.72), 1.056 (14.62), 1.074 (6.60), 1.111 (10.59), 1.228 (0.50), 1.234 (0.50), 1.272 (0.68), 1.356 (3.62), 1.367 (9.35), 1.387 (13.19), 1.853 (0.46), 1.886 (0.43), 2.081 (11.55), 3.195 (0.87), 3.243 (0.41), 3.256 (0.46), 3.269 (0.46), 3.281 (0.46), 3.415 (2.96), 3.432 (8.65), 3.449 (6.98), 3.467 (2.59), 3.692 (0.46), 3.719 (0.43), 5.203 (2.88), 5.230 (1.05), 5.253 (0.66).

### Intermediate 9

### (rac)-tert-butyl-3-(2-hydroxyethyl)-3-(3-hydroxy-1H-pyrazol-5-yl)pyrrolidine-1-carboxylate

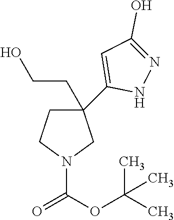 [SMILES: CC(C)(C)OC(=O)N1CCC(CCO)(c2cc(O)n[nH]2)C1]

### (rac)-tert-butyl-3-(2-{[tert-butyl(dimethyl)silyl]oxy}ethyl)-3-(5-hydroxy-1H-pyrazol-3-yl)pyrrolidine-1-carboxylate

(243 mg, 590 μmol) was solubilised in methanol (1.1 mL), hydrochloride (440 μL, 4.0 M in dioxane, 1.8 mmol) was added and the mixture was stirred for 2 h at rt under argon. Potassium carbonate (490 mg, 3.54 mmol) and di-tert-butyl dicarbonate (200 μL, 890 μmol) were added and the mixture was stirred for 1 h at rt under argon. The mixture was filtered and evaporated to give the title compound.

### Intermediate 10

### (rac)-tert-butyl-2′-hydroxy-5′,6′-dihydrospiro[pyrrolidine-3,4′-pyrrolo[1,2-b]pyrazole]-1-carboxylate

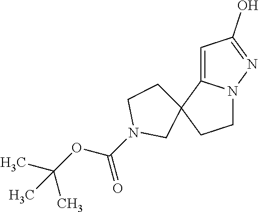 [SMILES: CC(C)(C)OC(=O)N1CCC2(CCn3nc(O)cc32)C1]

(Rac)-tert-butyl-3-(2-hydroxyethyl)-3-(3-hydroxy-1H-pyrazol-5-yl)pyrrolidine-1-carboxylate (5.24 g, 17.6 mmol) and triphenylphosphine (11.09 g, 42.3 mmol) were solubilised in THF (82 mL) under argon, DIAD (8.0 mL) was added dropwise and the mixture was stirred for 4 h at rt. The mixture was evaporated and purified by flash chromatography to give 2.83 g (95% purity, 55% yield) of the target compound.

¹H-NMR (400 MHz, DMSO-d6) δ[ppm]: 1.055 (0.45), 1.391 (16.00), 1.418 (13.84), 1.936 (0.55), 1.950 (0.82), 1.964 (0.84), 1.982 (0.88), 2.008 (0.64), 2.325 (0.40), 2.329 (0.55), 2.334 (0.43), 2.347 (1.09), 2.363 (1.82), 2.381 (1.13), 2.521 (1.64), 2.525 (1.12), 2.671 (0.43), 3.365 (0.83), 3.426 (0.74), 3.434 (0.63), 3.441 (0.65), 3.454 (0.50), 3.913 (1.22), 3.918 (1.44), 3.931 (2.38), 3.935 (2.82), 3.947 (1.19), 3.952 (1.45), 5.208 (10.10), 9.584 (1.93).

### Intermediate 11

### (rac)-tert-butyl-2′-[(trifluoromethanesulfonyl)oxy]-5′,6′-dihydrospiro[pyrrolidine-3,4′-pyrrolo[1,2-b]pyrazole]-1-carboxylate

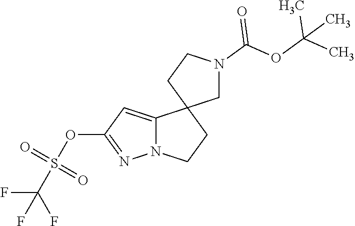 [SMILES: CC(C)(C)OC(=O)N1CCC2(CCn3nc(OS(=O)(=O)C(F)(F)F)cc32)C1]

(Rac)-tert-butyl-2′-hydroxy-5′,6′-dihydrospiro[pyrrolidine-3,4′-pyrrolo[1,2-b]pyrazole]-1-carboxylate (3.30 g, 11.8 mmol, 22% purity) was dissolved in THF (100 mL) under argon and treated with N,N-diisopropylethylamine (10 mL, 59 mmol) then N,N-bis(trifluoromethanesulfonyl)aniline (6.33 g, 17.7 mmol) was added and stirred at 60° C. for 1 h. The reaction mixture was concentrated and the residue was purified by flash chromatography to afford 600 mg (56% yield) of the title compound.

1H-NMR (400 MHz, DMSO-d6) δ[ppm]: 0.000 (12.78), 1.156 (1.57), 1.173 (5.35), 1.188 (5.10), 1.192 (2.37), 1.231 (0.58), 1.235 (0.48), 1.248 (0.83), 1.265 (0.42), 1.394 (16.00), 1.422 (12.77), 1.989 (4.21), 2.032 (0.58), 2.048 (0.79), 2.060 (0.85), 2.077 (1.07), 2.097 (0.81), 2.108 (0.44), 2.121 (0.86), 2.471 (1.67), 2.520 (1.72), 2.525 (1.11), 3.371 (0.69), 3.379 (0.88), 3.397 (0.97), 3.428 (4.90), 3.455 (0.50), 3.462 (0.81), 3.474 (0.88), 3.482 (1.12), 3.489 (0.76), 3.494 (0.94), 3.501 (0.68), 3.509 (0.66), 3.521 (0.49), 4.020 (0.97), 4.037 (0.96), 4.191 (0.42), 4.201 (1.17), 4.212 (1.51), 4.217 (1.63), 4.220 (1.58), 4.229 (1.66), 4.232 (1.87), 4.237 (1.32), 4.248 (1.33), 4.258 (0.51), 4.768 (0.45), 6.255 (2.91), 8.883 (0.91).

### Intermediate 12

### (rac)-tert-butyl-3-[3-bromo-1-(2-hydroxyethyl)-1H-pyrazol-5-yl]-3-hydroxypyrrolidine-1-carboxylate

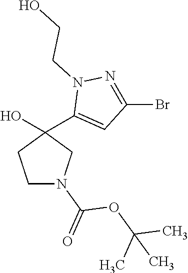 [SMILES: CC(C)(C)OC(=O)N1CCC(O)(c2cc(Br)nn2CCO)C1]

2-(3-Bromo-1H-pyrazol-1-yl)ethan-1-ol (1000 mg, 5.23 mmol) was solubilised in THF (25 mL), cooled to −78° C. and LDA (5.2 mL, 2.0 M in THF/heptane/ethylbenzene, 10 mmol) was added dropwise. The mixture was allowed to warm up to −20° C. and stirred at −20° C. for 45 min. The mixture was cooled to −78° C., tert-butyl 3-oxopyrrolidine-1-carboxylate (808 mg, 4.36 mmol) solubilised in THF (10 mL) was added dropwise and the mixture was stirred for 1 h at −78° C. The mixture was allowed to warm up to rt overnight. The mixture was quenched with saturated sodium bicarbonate solution, extracted with ethyl acetate, washed with brine, dried and concentrated. The residue was purified by flash chromatography to give 622 mg (38% yield) of the target compound.

¹H-NMR (400 MHz, DMSO-d6) δ[ppm]: 1.398 (13.16), 1.408 (16.00), 2.173 (0.49), 2.196 (0.95), 2.219 (0.73), 2.518 (1.49), 2.523 (0.95), 3.375 (1.23), 3.388 (1.14), 3.396 (1.15), 3.409 (0.63), 3.424 (0.69), 3.454 (0.83), 3.481 (0.44), 3.509 (0.74), 3.574 (0.63), 3.623 (0.63), 3.651 (0.48), 3.728 (0.61), 3.742 (1.78), 3.757 (1.87), 3.771 (0.70), 4.288 (1.23), 4.303 (1.39), 4.320 (0.55), 4.944 (0.81), 4.953 (0.98), 4.966 (0.41), 5.759 (0.96), 5.871 (1.43), 5.885 (1.12), 6.315 (4.60).

### Intermediate 13

### (rac)-tert-butyl-2-bromo-6,7-dihydrospiro[pyrazolo[5,1-c][1,4]oxazine-4,3′-pyrrolidine]-1′-carboxylate

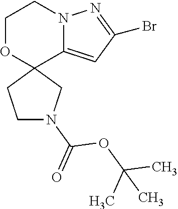 [SMILES: CC(C)(C)OC(=O)N1CCC2(C1)OCCn1nc(Br)cc12]

(Rac)-tert-butyl-3-[3-bromo-1-(2-hydroxyethyl)-1H-pyrazol-5-yl]-3-hydroxypyrrolidine-1-carboxylate (100 mg, 266 μmol) and triphenylphosphine (97.6 mg, 372 μmol) were solubilised in THF (1.5 mL), DIAD (68 μL) was added dropwise and the mixture was stirred at rt overnight. The mixture was evaporated and purified by flash chromatography to give 84.0 mg (88% yield) of the target compound.

¹H-NMR (400 MHz, DMSO-d6) δ[ppm]: 1.170 (2.41), 1.186 (2.37), 1.392 (16.00), 1.411 (14.84), 2.255 (0.63), 2.266 (0.56), 2.322 (0.56), 2.326 (0.71), 2.331 (0.52), 2.518 (2.60), 2.522 (1.67), 2.664 (0.52), 2.669 (0.72), 2.673 (0.52), 3.312 (0.96), 3.355 (0.78), 3.375 (0.84), 3.405 (0.89), 3.426 (0.76), 3.455 (0.93), 3.471 (0.65), 3.495 (1.07), 3.518 (0.51), 3.664 (0.60), 3.691 (0.98), 3.719 (0.46), 4.071 (11.76), 5.759 (0.96), 6.480 (2.27), 6.492 (2.37), 8.882 (0.44).

### Intermediate 14

### (rac)-tert-butyl-2-(6-amino-5-cyanopyridin-3-yl)-6,7-dihydrospiro[pyrazolo[5,1-c][1,4]oxazine-4,3′-pyrrolidine]-1′-carboxylate

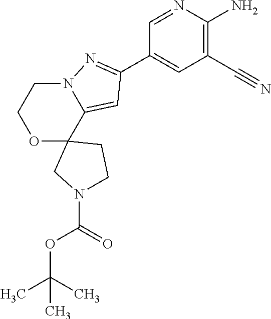 [SMILES: CC(C)(C)OC(=O)N1CCC2(C1)OCCn1nc(-c3cnc(N)c(C#N)c3)cc12]

(Rac)-tert-butyl-2-bromo-6,7-dihydrospiro[pyrazolo[5,1-c][1,4]oxazine-4,3′-pyrrolidine]-1′-carboxylate (621 mg, 1.73 mmol), 2-amino-5-(4,4,5,5-tetramethyl-1,3,2-dioxaborolan-2-yl)pyridine-3-carbonitrile (850 mg, 3.47 mmol), XPhos Pd G2 (68.2 mg, 86.7 μmol) and potassium phosphate (10 mL, 0.50 M, 5.2 mmol) were stirred in degassed dioxane (25 mL) for 2 h at 100° C. The reaction mixture was diluted with ethyl acetate and water. The phases were separated and the aqueous phase was extracted twice with ethyl acetate. The combined organic phases were dried over sodium sulfate and concentrated. The residue was purified by flash chromatography to afford 736 mg of the title compound.

¹H-NMR (400 MHz, DMSO-d6) δ[ppm]: 1.065 (4.11), 1.265 (0.50), 1.399 (7.96), 1.424 (6.94), 2.518 (0.73), 2.523 (0.49), 3.159 (16.00), 3.172 (15.27), 3.366 (0.44), 3.375 (0.41), 3.397 (0.49), 3.428 (0.42), 3.483 (0.44), 3.551 (0.65), 3.726 (0.52), 3.941 (0.69), 4.087 (1.54), 4.100 (4.02), 4.113 (6.91), 4.126 (1.93), 6.699 (0.95), 6.717 (1.09), 7.046 (2.34), 8.114 (1.17), 8.119 (1.15), 8.591 (2.11), 8.598 (2.12).

### Intermediate 15

### (rac)-tert-butyl-2′-(6-amino-5-cyanopyridin-3-yl)-5′,6′-dihydrospiro[pyrrolidine-3,4′-pyrrolo[1,2-b]pyrazole]-1-carboxylate

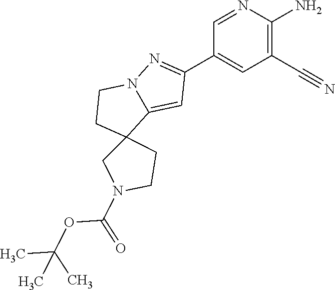 [SMILES: CC(C)(C)OC(=O)N1CCC2(CCn3nc(-c4cnc(N)c(C#N)c4)cc32)C1]

(Rac)-tert-butyl-2′-[(trifluoromethanesulfonyl)oxy]-5′,6′-dihydrospiro[pyrrolidine-3,4′-pyrrolo[1,2-b]pyrazole]-1-carboxylate (713 mg, 1.73 mmol), 2-amino-5-(4,4,5,5-tetramethyl-1,3,2-dioxaborolan-2-yl)pyridine-3-carbonitrile (850 mg, 3.47 mmol), XPhos Pd G2 (68.2 mg, 86.7 μmol) and potassium phosphate (10 mL, 0.50 M, 5.2 mmol) were dissolved in degassed dioxane (25 mL) under argon and stirred at 100° C. for 2 h. The mixture was diluted with ethyl acetate and water, the layers were separated and the aqueous phase was extracted twice with ethyl acetate. The combined organic phases were dried over sodium sulfate and concentrated. Purified by flash chromatography to afford 730 mg of the title compound.

¹H-NMR (400 MHz, DMSO-d6) δ[ppm]: 1.065 (2.09), 1.396 (7.48), 1.432 (6.06), 2.029 (0.79), 2.047 (0.64), 2.518 (0.66), 2.523 (0.65), 2.534 (0.65), 2.542 (0.75), 2.560 (0.48), 3.159 (16.00), 3.172 (15.80), 3.386 (0.65), 3.407 (2.03), 3.434 (0.62), 3.496 (0.64), 4.087 (1.45), 4.101 (3.63), 4.113 (3.70), 4.127 (1.34), 4.154 (0.51), 4.163 (0.62), 4.172 (0.92), 4.181 (0.90), 4.189 (0.62), 4.198 (0.49), 6.454 (0.82), 6.479 (1.01), 6.985 (2.41), 8.151 (1.16), 8.156 (1.10), 8.611 (1.97), 8.618 (1.97).

### Intermediate 16

### (rac)-tert-butyl-2′-[6-amino-5-(trifluoromethyl)pyridin-3-yl]-5′,6′-dihydrospiro[pyrrolidine-3,4′-pyrrolo[1,2-b]pyrazole]-1-carboxylate

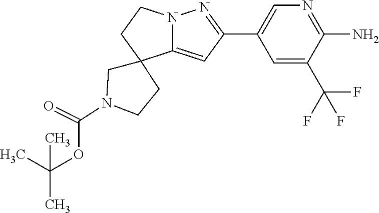 [SMILES: CC(C)(C)OC(=O)N1CCC2(CCn3nc(-c4cnc(N)c(C(F)(F)F)c4)cc32)C1]

(Rac)-tert-butyl-2′-[(trifluoromethanesulfonyl)oxy]-5′,6′-dihydrospiro[pyrrolidine-3,4′-pyrrolo[1,2-b]pyrazole]-1-carboxylate (2.50 g, 6.08 mmol) was solubilised in dioxane (51 mL) under nitrogen, 5-(4,4,5,5-tetramethyl-1,3,2-dioxaborolan-2-yl)-3-(trifluoromethyl)pyridin-2-amine (2.63 g, 9.12 mmol), potassium phosphate solution (36 mL, 0.50 M, 18 mmol) and XPhos Pd G2 (239 mg, 304 μmol) were added and the mixture was stirred overnight at 100° C. The mixture was diluted with ethyl acetate, washed with saturated sodium chloride solution, dried and concentrated under reduced pressure. The residue was purified by flash chromatography to give 2.70 g of the target compound.

¹H-NMR (400 MHz, DMSO-d6) δ[ppm]: 1.066 (16.00), 1.172 (0.51), 1.397 (3.81), 1.416 (0.58), 1.433 (2.99), 1.988 (1.01), 2.518 (0.54), 2.523 (0.45), 3.413 (1.28), 3.938 (2.53), 4.176 (0.46), 4.184 (0.45), 5.758 (1.10), 6.507 (0.50), 6.538 (1.08), 8.016 (0.55), 8.588 (0.61), 8.592 (0.59).

### Intermediate 17

### (rac)-tert-butyl-2-[6-amino-5-(trifluoromethyl)pyridin-3-yl]-6,7-dihydrospiro[pyrazolo[5,1-c][1,4]oxazine-4,3′-pyrrolidine]-1′-carboxylate

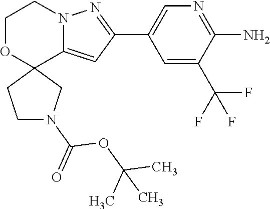 [SMILES: CC(C)(C)OC(=O)N1CCC2(C1)OCCn1nc(-c3cnc(N)c(C(F)(F)F)c3)cc12]

Intermediate 17 was prepared in analogy to intermediate (rac)-tert-butyl-2′-[6-amino-5-(trifluoromethyl)pyridin-3-yl]-5′,6′-dihydrospiro[pyrrolidine-3,4′-pyrrolo[1,2-b]pyrazole]-1-carboxylate using (rac)-tert-butyl-2-bromo-6,7-dihydrospiro[pyrazolo[5,1-c][1,4]oxazine-4,3′-pyrrolidine]-1′-carboxylate and 5-(4,4,5,5-tetramethyl-1,3,2-dioxaborolan-2-yl)-3-(trifluoromethyl)pyridin-2-amine.

### Intermediate 18

### (rac)-5-[5′,6′-dihydrospiro[pyrrolidine-3,4′-pyrrolo[1,2-b]pyrazol]-2′-yl]-3-(trifluoromethyl)pyridin-2-amine-hydrochloride

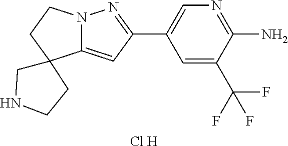 [SMILES: Nc1ncc(-c2cc3n(n2)CCC32CCNC2)cc1C(F)(F)F ; "Cl H"]

(Rac)-tert-butyl-2′-[6-amino-5-(trifluoromethyl)pyridin-3-yl]-5′,6′-dihydrospiro[pyrrolidine-3,4′-pyrrolo[1,2-b]pyrazole]-1-carboxylate (2.70 g, 6.38 mmol) was solubilised in dioxane (56 mL), hydrochloric acid (16 mL, 4.0 M in dioxane, 64 mmol) was added and the mixture was stirred overnight at rt. The mixture was evaporated to give 3.30 g of the target compound.

1H-NMR (400 MHz, DMSO-d6) δ[ppm]: 1.065 (16.00), 1.156 (0.82), 2.164 (0.59), 2.172 (0.41), 2.332 (0.74), 2.518 (3.21), 2.522 (1.93), 2.542 (0.80), 2.546 (0.76), 2.673 (1.08), 3.565 (3.33), 3.911 (3.01), 4.182 (0.43), 4.197 (0.56), 6.624 (2.53), 8.023 (0.74), 8.028 (0.72), 8.576 (0.74).

### Intermediate 19

### (rac)-5-[6,7-dihydrospiro[pyrazolo[5,1-c][1,4]oxazine-4,3′-pyrrolidin]-2-yl]-3-(trifluoromethyl)pyridin-2-amine-hydrochloride

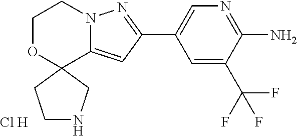

This intermediate was prepared in analogy to intermediate (rac)-5-[5′,6′-dihydrospiro[pyrrolidine-3,4′-pyrrolo[1,2-b]pyrazol]-2′-yl]-3-(trifluoromethyl)pyridin-2-amine-hydrochloride using (rac)-tert-butyl-2-[6-amino-5-(trifluoromethyl)pyridin-3-yl]-6,7-dihydrospiro[pyrazolo[5,1-c][1,4]oxazine-4,3′-pyrrolidine]-1′-carboxylate.

### Intermediate 20

### (rac)-2-amino-5-[6,7-dihydrospiro[pyrazolo[5,1-c][1,4]oxazine-4,3′-pyrrolidin]-2-yl]pyridine-3-carbonitrile hydrochloride

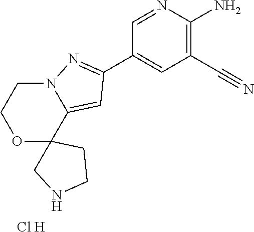

This intermediate was prepared in analogy to Intermediate (rac)-5-[5′,6′-dihydrospiro[pyrrolidine-3,4′-pyrrolo[1,2-b]pyrazol]-2′-yl]-3-(trifluoromethyl)pyridin-2-amine-hydrochloride using (rac)-tert-butyl-2-(6-amino-5-cyanopyridin-3-yl)-6,7-dihydrospiro[pyrazolo[5,1-c][1,4]oxazine-4,3′-pyrrolidine]-1′-carboxylate.

¹H-NMR (400 MHz, DMSO-d6) δ[ppm]: 1.064 (3.61), 1.268 (1.74), 2.212 (0.53), 2.235 (1.04), 2.245 (0.99), 2.272 (1.27), 2.297 (0.74), 2.518 (6.21), 2.523 (3.83), 3.164 (8.69), 3.250 (0.63), 3.269 (1.35), 3.282 (1.11), 3.301 (1.82), 3.319 (1.23), 3.338 (0.95), 3.488 (0.80), 3.498 (1.02), 3.565 (2.56), 3.684 (1.03), 3.698 (1.41), 3.713 (1.14), 3.729 (1.01), 3.989 (9.14), 4.088 (2.10), 4.152 (16.00), 6.768 (14.71), 8.166 (9.01), 8.172 (8.69), 8.602 (8.75), 8.607 (8.64), 9.839 (0.80).

### Intermediate 21

### (rac)-2-amino-5-[5′,6′-dihydrospiro[pyrrolidine-3,4′-pyrrolo[1,2-b]pyrazol]-2′-yl]pyridine-3-carbonitrile hydrochloride

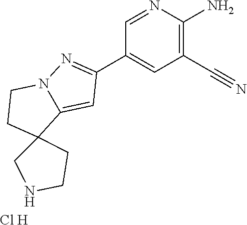

This intermediate was prepared in analogy to Intermediate (rac)-5-[5′,6′-dihydrospiro[pyrrolidine-3,4′-pyrrolo[1,2-b]pyrazol]-2′-yl]-3-(trifluoromethyl)pyridin-2-amine-hydrochloride using (rac)-tert-butyl-2′-(6-amino-5-cyanopyridin-3-yl)-5′,6′-dihydrospiro[pyrrolidine-3,4′-pyrrolo[1,2-b]pyrazole]-1-carboxylate.

¹H-NMR (400 MHz, DMSO-d6) δ[ppm]: 1.062 (1.01), 2.133 (0.79), 2.153 (1.92), 2.164 (1.30), 2.173 (1.17), 2.181 (1.08), 2.523 (0.88), 2.532 (0.52), 2.548 (0.68), 2.566 (0.94), 2.580 (1.01), 2.599 (0.58), 2.695 (0.56), 2.714 (1.00), 2.729 (0.90), 2.743 (0.54), 2.747 (0.63), 2.762 (0.41), 3.161 (16.00), 3.228 (0.66), 3.242 (0.71), 3.275 (0.47), 3.311 (0.41), 3.321 (0.47), 3.332 (0.64), 3.340 (0.54), 3.361 (0.40), 3.382 (0.90), 3.388 (0.66), 3.404 (1.08), 3.415 (1.30), 3.427 (1.04), 3.433 (1.00), 4.178 (1.12), 4.187 (1.19), 4.194 (1.57), 4.197 (1.63), 4.201 (1.66), 4.206 (1.51), 4.213 (1.19), 4.221 (0.99), 6.149 (0.46), 6.351 (0.50), 6.639 (8.60), 8.264 (4.17), 8.269 (4.21), 8.592 (4.68), 8.598 (4.48), 9.824 (0.61).

### Intermediate 22

### (rac)-5′,6′-dihydrospiro[pyrrolidine-3,4′-pyrrolo[1,2-b]pyrazol]-2′-yl trifluoromethanesulfonate-hydrogen chloride

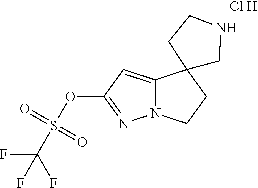

Tert-butyl 2′-{[(trifluoromethyl)sulfonyl]oxy}-5′,6′-dihydro-1H-spiro[pyrrolidine-3,4′-pyrrolo[1,2-b]pyrazole]-1-carboxylate (1.00 g, 2.43 mmol) was dissolved in dioxane (21 mL) and cooled to 0° C. Hydrochloric acid (3.04 mL, 12.15 mmol, 4M in dioxane) was added and the reaction mixture was stirred at rt overnight. The rection mixture was concentrated to afford 1.11 g of the title compound.

¹H-NMR (400 MHz, DMSO-d6) δ[ppm]: 1.391 (1.27), 1.418 (1.03), 2.161 (0.45), 2.164 (0.43), 2.180 (1.26), 2.199 (0.82), 2.518 (0.50), 2.524 (0.64), 2.539 (0.48), 2.652 (0.41), 3.307 (0.49), 3.390 (0.49), 3.400 (1.01), 3.429 (0.61), 3.564 (16.00), 4.210 (0.56), 4.218 (0.61), 4.226 (0.78), 4.228 (0.78), 4.232 (0.67), 4.238 (0.61), 4.245 (0.62), 4.252 (0.47), 6.444 (2.92), 9.626 (0.44).

### Intermediate 23

### (rac)-1-[(1-phenylcyclobutyl)carbamoyl]-5′,6′-dihydrospiro[pyrrolidine-3,4′-pyrrolo[1,2-b]pyrazol]-2′-yl trifluoromethanesulfonate

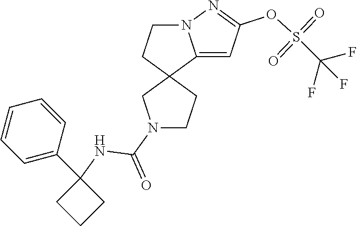 [SMILES: O=C(NC1(c2ccccc2)CCC1)N1CCC2(CCn3nc(OS(=O)(=O)C(F)(F)F)cc32)C1]

1-Phenylcyclobutan-1-amine (429 mg, 2.92 mmol), N,N′-carbonyldiimidazole (473 mg, 2.92 mmol) and N,N-diisopropylethylamine (2.1 ml, 12 mmol) were dissolved in DMF (25 mL). It was stirred for 1 h at rt. Then (rac)-5′,6′-dihydrospiro[pyrrolidine-3,4′-pyrrolo[1,2-b]pyrazol]-2′-yl trifluoromethanesulfonate-hydrogen chloride (845 mg, 2.43 mmol) in DMF (10 mL) was added and stirred at 60° C. for 3 h. Half concentrated sodium chloride solution was added and the mixture was extracted three times with ethyl acetate. The combined organic phases were dried and concentrated obtaining 1.55 g of the title compound.

¹H-NMR (400 MHz, DMSO-d6) δ[ppm]: 1.392 (2.75), 1.420 (2.20), 1.739 (0.41), 1.762 (0.63), 1.780 (0.60), 1.785 (0.59), 1.950 (0.50), 1.957 (0.51), 1.972 (0.74), 1.978 (0.60), 1.988 (0.44), 1.995 (0.57), 2.000 (0.58), 2.011 (0.42), 2.023 (0.47), 2.042 (0.73), 2.055 (0.69), 2.060 (0.67), 2.076 (0.66), 2.097 (1.02), 2.117 (0.52), 2.128 (0.47), 2.327 (0.81), 2.332 (0.60), 2.344 (1.28), 2.349 (1.21), 2.353 (1.12), 2.358 (1.13), 2.366 (1.44), 2.380 (1.43), 2.397 (1.02), 2.419 (0.67), 2.438 (0.70), 2.453 (1.77), 2.466 (2.62), 2.472 (2.45), 2.518 (1.05), 2.523 (0.72), 2.728 (13.33), 2.730 (13.09), 2.888 (16.00), 3.382 (0.83), 3.404 (0.54), 3.430 (2.61), 3.436 (2.45), 3.461 (0.46), 3.475 (0.46), 3.480 (0.44), 3.487 (0.56), 3.495 (0.60), 3.501 (0.54), 3.507 (0.60), 3.513 (0.42), 4.207 (1.69), 4.214 (0.70), 4.225 (2.94), 4.235 (0.64), 4.243 (1.59), 6.186 (4.87), 6.253 (0.51), 6.410 (0.71), 6.713 (2.26), 7.012 (1.62), 7.138 (0.73), 7.142 (0.46), 7.147 (0.47), 7.152 (0.57), 7.156 (1.71), 7.164 (1.03), 7.171 (0.78), 7.174 (1.27), 7.178 (0.78), 7.183 (0.81), 7.186 (0.44), 7.254 (1.09), 7.257 (0.73), 7.263 (2.01), 7.267 (0.98), 7.270 (1.03), 7.273 (2.14), 7.282 (3.26), 7.291 (1.47), 7.296 (0.90), 7.300 (1.88), 7.336 (1.74), 7.339 (1.95), 7.352 (0.44), 7.357 (1.32), 7.361 (0.94), 7.406 (2.77), 7.409 (2.96), 7.422 (0.74), 7.427 (2.42), 7.430 (1.93), 7.639 (1.02), 7.951 (1.94).

### Intermediate 24

### tert-butyl (3S)-2′-[(trifluoromethanesulfonyl)oxy]-5′,6′-dihydrospiro[pyrrolidine-3,4′-pyrrolo[1,2-b]pyrazole]-1-carboxylate

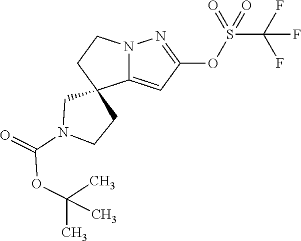 [SMILES: CC(C)(C)OC(=O)N1CC[C@]2(CCn3nc(OS(=O)(=O)C(F)(F)F)cc32)C1]

The title compound was prepared by chiral separation of the corresponding racemic mixture.

Chiral Separation Protocol:

Preparative

Instrument: Sepiatec: Prep SFC100; Column: Chiralpak IG 5μ 250×30 mm; eluent A: CO2; eluent B: ethanol+0.2 vol % aqueous ammonia (32%); isocratic: 5% B; flow: 100 mL/min; temperature: 40° C.; BPR: 150 bar; UV: 210 nm

Analytical

Instrument: Agilent: 1260, Aurora SFC-Modul; Column: Chiralpak IG 5μ 100×4.6 mm; eluent A: CO2; eluent B: ethanol+0.2 vol % aqueous ammonia (32%); isocratic: 5% B; flow: 4 mL/min; temperature: 37.5° C.; BPR: 100 bar; UV: 210 nm

Retention time preparative method: 6.0-7.5 min

Retention Time analytical method: 1.60 min

[α]²⁰_{D}: −9.6° (c=1.00, DMSO)

### Intermediate 25

### tert-butyl (3R)-2′-[(trifluoromethanesulfonyl)oxy]-5′,6′-dihydrospiro[pyrrolidine-3,4′-pyrrolo[1,2-b]pyrazole]-1-carboxylate

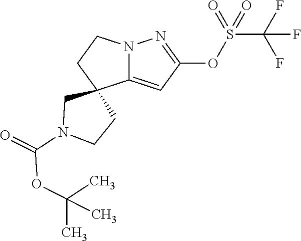 [SMILES: CC(C)(C)OC(=O)N1CC[C@@]2(CCn3nc(OS(=O)(=O)C(F)(F)F)cc32)C1]

The title compound was prepared by chiral separation of the corresponding racemic mixture.

Chiral Separation Protocol:

Preparative

Instrument: Sepiatec: Prep SFC100; Column: Chiralpak IG 5μ 250×30 mm; eluent A: CO2; eluent B: ethanol+0.2 vol % aqueous ammonia (32%); isocratic: 5% B; flow: 100 mL/min; temperature: 40° C.; BPR: 150 bar; UV: 210 nm

Analytical

Instrument: Agilent: 1260, Aurora SFC-Modul; Column: Chiralpak IG 5μ 100×4.6 mm; eluent A: CO2; eluent B: ethanol+0.2 vol % aqueous ammonia (32%); isocratic: 5% B; flow: 4 mL/min; temperature: 37.5° C.; BPR: 100 bar; UV: 210 nm

Retention time preparative method: 3.8-4.7 min

Retention Time analytical method: 1.05 min

[α]²⁰_{D}: +10.4° (c=1.00, DMSO)

### Intermediate 26

### (3R)-5′,6′-dihydrospiro[pyrrolidine-3,4′-pyrrolo[1,2-b]pyrazol]-2′-yl trifluoromethanesulfonate-hydrochloride salt

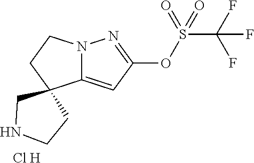

tert-butyl (3R)-2′-[(trifluoromethanesulfonyl)oxy]-5′,6′-dihydrospiro[pyrrolidine-3,4′-pyrrolo[1,2-b]pyrazole]-1-carboxylate (5.00 g, 12.2 mmol) was solubilised in 1,4-dioxane (200 mL) and treated with HCl in dioxane (30 mL, 4.0 M, 120 mmol). The reaction mixture was stirred for 8 h at 40° C. The reaction was then cooled to rt and concentrated under reduced pressure to give 4.69 g (90% purity, 100% yield) of the title compound that was used without further purification.

LC-MS (method 1): Rₜ=1.00 min; MS (ESIpos): m/z=312 [M+H]⁺

### Intermediate 27

### (rac)-1-(ethylcarbamoyl)-5′,6′-dihydrospiro[pyrrolidine-3,4′-pyrrolo[1,2-b]pyrazol]-2′-yl trifluoromethanesulfonate

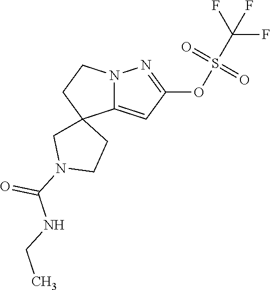 [SMILES: CCNC(=O)N1CCC2(CCn3nc(OS(=O)(=O)C(F)(F)F)cc32)C1]

(rac)-5′,6′-dihydrospiro[pyrrolidine-3,4′-pyrrolo[1,2-b]pyrazol]-2′-yl trifluoromethanesulfonate (3.00 g, 9.64 mmol) was solubilised in dichloromethane (110 mL), N,N-diisopropylethylamine (17 mL, 96 mmol) was added, cooled to 0° C. and isocyanatoethane (790 μL, 9.6 mmol) was added. The mixture was stirred for 2 h at rt. It was concentrated under reduced pressure and purified by flash chromatography to give 2.80 g (76% yield) of the title compound.

LC-MS (method 1): Rₜ=1.04 min; MS (ESIpos): m/z=383 [M+H]⁺

1H-NMR (400 MHz, DMSO-d6): δ [ppm]=1.01 (t, 3H), 2.00-2.16 (m, 2H), 2.43-2.48 (m, 2H), 2.99-3.09 (m, 2H), 3.35-3.51 (m, 4H), 4.22 (t, 2H), 6.16 (t, 1H), 6.24 (s, 1H).

### Intermediate 28

### (3R)-1-(ethylcarbamoyl)-5′,6′-dihydrospiro[pyrrolidine-3,4′-pyrrolo[1,2-b]pyrazol]-2′-yl trifluoromethanesulfonate

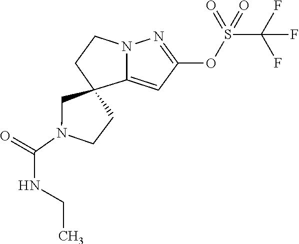 [SMILES: CCNC(=O)N1CC[C@@]2(CCn3nc(OS(=O)(=O)C(F)(F)F)cc32)C1]

(3R)-5′,6′-dihydrospiro[pyrrolidine-3,4′-pyrrolo[1,2-b]pyrazol]-2′-yl trifluoromethanesulfonate-hydrochloride salt (4.49 g, 90% purity, 11.6 mmol) was solubilised in dichloromethane (200 mL), N,N-diisopropylethylamine (20 mL, 120 mmol) was added and the mixture was cooled to 0° C. Ethylisocyanate (1.1 mL, 14 mmol) was added and the mixture was allowed to warm up to rt for 2 h. The mixture was concentrated under reduced pressure and the residue was purified by flash chromatography to give 4.12 g (98% purity, 91% yield) of the title compound.

1H-NMR (400 MHz, CHLOROFORM-d): δ [ppm]=1.16 (t, 3H), 2.09 (ddd, 1H), 2.18-2.28 (m, 1H), 2.45-2.54 (m, 1H), 2.54-2.64 (m, 1H), 3.30 (qd, 2H), 3.47-3.63 (m, 4H), 4.17 (br t, 1H), 4.23 (t, 2H), 5.86 (s, 1H).

[α]²⁰_{D}: +19.4° (c=1.00, MeOH)

LC-MS (method 1): Rₜ=1.02 min; MS (ESIpos): m/z=383 [M+H]⁺

### Intermediate 29

### (3R)-1-[(propan-2-yl)carbamoyl]-5′,6′-dihydrospiro[pyrrolidine-3,4′-pyrrolo[1,2-b]pyrazol]-2′-yl trifluoromethanesulfonate

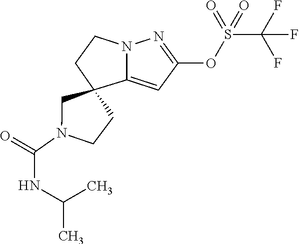 [SMILES: CC(C)NC(=O)N1CC[C@@]2(CCn3nc(OS(=O)(=O)C(F)(F)F)cc32)C1]

(3R)-5′,6′-dihydrospiro[pyrrolidine-3,4′-pyrrolo[1,2-b]pyrazol]-2′-yl trifluoromethanesulfonate hydrogen chloride salt (200 mg, 80% purity, 460 μmol) was solubilised in dichloromethane (7.9 mL), N,N-diisopropylethylamine (800 μl, 4.6 mmol) was added and the mixture was cooled to 0° C. isopropylisocyanate (54 μl, 550 μmol) was added slowly and the mixture allowed to warm up to r.t. After 2 h isopropylisocyanate (54 μl, 550 μmol) was added again and the reaction was stirred at rt for 18 h. The reaction mixture was then concentrated under reduced pressure and purified by flash column chromatography to give 186 mg (95% purity, 97% yield) of the title compound.

LC-MS (method 1): Rₜ=1.10 min; MS (ESIpos): m/z=397 [M+H]⁺

1H-NMR (400 MHz, CHLOROFORM-d): δ [ppm]=1.15-1.21 (m, 6H), 2.09 (ddd, 1H), 2.23 (dt, 1H), 2.45-2.54 (m, 1H), 2.55-2.63 (m, 1H), 3.46-3.54 (m, 3H), 3.57 (dd, 1H), 3.98 (br d, 2H), 4.24 (t, 2H), 5.86 (s, 1H).

### Intermediate 30

### (3R)-1-(cyclobutylcarbamoyl)-5′,6′-dihydrospiro[pyrrolidine-3,4′-pyrrolo[1,2-b]pyrazol]-2′-yl trifluoromethanesulfonate

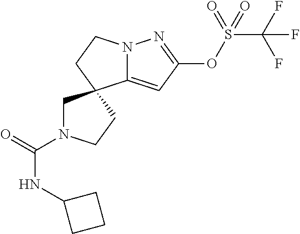 [SMILES: O=C(NC1CCC1)N1CC[C@@]2(CCn3nc(OS(=O)(=O)C(F)(F)F)cc32)C1]

(3R)-5′,6′-dihydrospiro[pyrrolidine-3,4′-pyrrolo[1,2-b]pyrazol]-2′-yl trifluoromethanesulfonate hydrogen chloride salt (430 mg, 80% purity, 989 μmol) was suspended in dichloromethane (17 mL), N,N-diisopropylethylamine (1.7 mL, 9.9 mmol) was added and the mixture was cooled to 0° C. Cyclobutylisocyanate (110 μl, 1.2 mmol) was added and the mixture allowed to warm up to r.t. After 2 h cyclobutylisocyanate (65 μl, 0.6 mmol) was added and the reaction was stirred 18 h at r.t. The reaction mixture was concentrated under reduced pressure and purified by dlash column chromatography to give 461 mg (90% purity, 103% yield) of the title compound.

LC-MS (method 1): Rₜ=1.12 min; MS (ESIpos): m/z=409 [M+H]⁺

1H-NMR (400 MHz, CHLOROFORM-d): δ [ppm]=1.63-1.73 (m, 2H), 1.76-1.89 (m, 2H), 2.09 (s, 1H), 2.18-2.27 (m, 1H), 2.31-2.41 (m, 2H), 2.50 (dd, 1H), 2.54-2.64 (m, 1H), 3.47-3.63 (m, 4H), 4.23 (t, 2H), 4.28-4.38 (m, 2H), 5.86 (s, 1H).

### Intermediate 31

### 1-tert-butyl 3-methyl 3-(prop-2-en-1-yl)azetidine-1,3-dicarboxylate

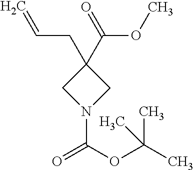 [SMILES: C=CCC1(C(=O)OC)CN(C(=O)OC(C)(C)C)C1]

To a solution of 1-tert-butyl 3-methyl azetidine-1,3-dicarboxylate (940 g, 4.37 mol, 1.00 equiv, CAS-RN [610791-05-4]) in dry THF (9 L) was added LiHMDS (8.8 L, 8.74 mol, 2.00 equiv) at −78° C. under nitrogen atmosphere, and the reaction mixture was stirred at this temperature for 30 min. Then a solution of 3-bromoprop-1-ene (1058 g, 8.74 mol, 2.00 equiv) in dry THF (5 L) was added and the mixture was left to warm to ambient temperature and stirred overnight (monitored by TLC). Sat. aq. solution of NH₄Cl (15 L) was added and the mixture was extracted with 3×10 L of EtOAc, and the combined organic layer was washed with 3×15 L of brine, dried over anhydrous Na₂SO₄, and concentrated under vacuum to yield a crude product which was directly purified by silica gel column (EtOAc/petroleum ether) to give 832 g (75%) of the title compound as yellow oil.

### Intermediate 32

### tert-butyl 3-(3-methoxy-3-oxopropanoyl)-3-(prop-2-en-1-yl)azetidine-1-carboxylate

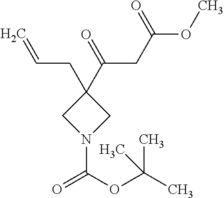 [SMILES: C=CCC1(C(=O)CC(=O)OC)CN(C(=O)OC(C)(C)C)C1]

To a solution of methyl acetate (482 g, 6.52 mol, 2.00 equiv) in dry THF (9 L) was added LiHMDS (6.6 L, 6.52 mol, 2.00 equiv) at −78° C. under nitrogen atmosphere, and the reaction mixture was stirred at this temperature for 1 h. Then a solution of 1-tert-butyl 3-methyl 3-(prop-2-en-1-yl)azetidine-1,3-dicarboxylate (832 g, 3.26 mol, 1.00 equiv) in dry THF (2 L) was added and the mixture was left to warm to rt and stirred for another 2 h (monitored by TLC). Sat. aq. NH4Cl (10 L) was added and the mixture was extracted with 3×10 L of EtOAc, and the combined organic layer was washed with 3×10 L of brine, dried over anhydrous Na2SO4, and concentrated under vacuum to yield a crude product which was directly purified by silica gel column (EtOAc/petroleum ether) to give 810 g (84%) of the title compound as yellow oil.

### Intermediate 33

### tert-butyl 3-(3-hydroxy-1H-pyrazol-5-yl)-3-(prop-2-en-1-yl)azetidine-1-carboxylate

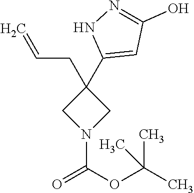 [SMILES: C=CCC1(c2cc(O)n[nH]2)CN(C(=O)OC(C)(C)C)C1]

To a stirred solution of tert-butyl 3-(3-methoxy-3-oxopropanoyl)-3-(prop-2-en-1-yl)azetidine-1-carboxylate (810 g, 2.73 mol, 1.00 equiv) in anhydrous EtOH (3 L) was added hydrazine-water (1:1) (280 g, 8.75 mol, 3.00 equiv). This mixture was stirred at an oil bath. The temperature was warmed to 80° C. and stirred for another 1 h (monitored by LCMS). The solvent was removed in vacuum and the residue was dissolved in 2 L EtOAc and washed with 2×2 L of aq. HCl (1 M), 1×2 L of brine, dried over anhydrous Na2SO4 to afford 779 g (96%) of the title compound as white solid. LCMS: (ES, m/z): 280 [M+H]⁺

### Intermediate 34

### tert-butyl 3-(prop-2-en-1-yl)-3-[3-(trifluoromethanesulfonyloxy)-1H-pyrazol-5-yl]azetidine-1-carboxylate

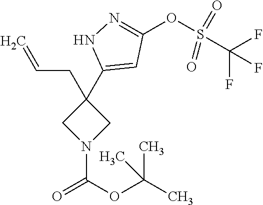 [SMILES: C=CCC1(c2cc(OS(=O)(=O)C(F)(F)F)n[nH]2)CN(C(=O)OC(C)(C)C)C1]

To a stirred solution of tert-butyl 3-(3-hydroxy-1H-pyrazol-5-yl)-3-(prop-2-en-1-yl)azetidine-1-carboxylate (779 g, 2.79 mol, 1.00 equiv) in anhydrous THF (10 L) was added DIPEA (1079 g, 8.37 mol, 3.00 equiv) followed by 1,1,1-trifluoro-N-phenyl-N-trifluoromethanesulfonylmethanesulfonamide (1095 g, 3.07 mol, 1.10 equiv). This mixture was stirred at 60° C. in an oil bath for 2 h, the solvent was removed under vacuum and the residue was dissolved in 2 L EtOAc and washed with 3×2 L of aq. HCl (1 M) and 3×2 L of water, dried over anhydrous Na2SO4, concentrated under vacuum and recrystallization from MTBE to afford 810 g (71%) of the title compound as white solid. LCMS: (ES, m/z): 412 [M+H]⁺

### Intermediate 35

### tert-butyl 3-(2-hydroxyethyl)-3-[3-(trifluoromethanesulfonyloxy)-1H-pyrazol-5-yl]azetidine-1-carboxylate

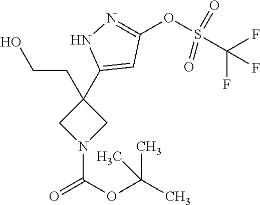 [SMILES: CC(C)(C)OC(=O)N1CC(CCO)(c2cc(OS(=O)(=O)C(F)(F)F)n[nH]2)C1]

To a stirred solution of tert-butyl 3-(prop-2-en-1-yl)-3-[3-(trifluoromethanesulfonyloxy)-1H-pyrazol-5-yl]azetidine-1-carboxylate (400 g, 0.97 mol, 1.00 equiv) in dioxane/H₂O (2 L, 3/2, v/v) was added 2,6-Lutidine (207 g, 1.94 mol, 2.00 equiv) followed by K20sO4*2H2O (7.87 g, 0.02 mol, 0.02 equiv). This mixture was stirred at rt for 40 min before NalO4 (888 g, 4.15 mol, 4.00 equiv) was added in batches. The final mixture was stirred for another 2 h at rt. The reaction mixture was filtrated and the filtrate cake was washed with EtOAc (4 L), the filtrate was separated. The organic layer was concentrated under vacuum. The residue was dissolved in 2 L MeOH and stirred at 0° C. in an ice-water bath, then NaBH4 (19.0 g, 0.51 mol, 0.50 equiv) was added in small portions. The pH value of the reaction mixture was adjusted to 7 by 0.1 mol/L HCl aq. solution when the addition was completed. The resulting mixture was extracted with 3×5 L of DCM. The organic phase was combined, washed with 3×5 L of brine, dried over anhydrous Na2SO4, concentrated under vacuum yield a crude product which was directly purified by silica gel column (EtOAc/petroleum ether) to give 360 g (89.4%) of the title compound as yellow oil. LCMS: (ES, m/z): 416 [M+H]+

### Intermediate 36

### tert-butyl 2′-(trifluoromethanesulfonyloxy)-5′,6′-dihydrospiro[azetidine-3,4′-pyrrolo[1,2-b]pyrazole]-1-carboxylate

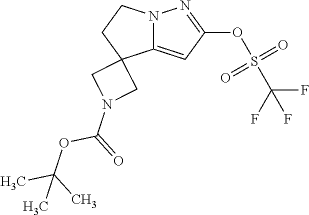 [SMILES: CC(C)(C)OC(=O)N1CC2(CCn3nc(OS(=O)(=O)C(F)(F)F)cc32)C1]

To a solution of tert-butyl 3-(2-hydroxyethyl)-3-[3-(trifluoromethanesulfonyloxy)-1H-pyrazol-5-yl]azetidine-1-carboxylate (360 g, 0.87 mol, 1.00 equiv) and PPh3 (320 g, 1.22 mol, 1.40 equiv) in dry THF (4 L) was added DIAD (246 g, 1.4 mol, 1.30 equiv) dropwise at rt under nitrogen atmosphere. The reaction mixture was stirred at this temperature for 16 h. The solvent was removed under vacuum and the residue was dissolved in MTBE (2 L) and stirred overnight until white solid was precipitated. The solid was removed by filtration. The filtrate was concentrated under vacuum and the crude product was re-crystallized from MeOH to give 240 g (69.5%) of the title compound as white solid. LCMS: (ES, m/z): 398 [M+H]+, 1H-NMR (400 MHz, DMSO-d6) δ[ppm]: 1.399 (16.00), 2.787 (0.67), 2.805 (1.02), 2.822 (0.71), 4.044 (1.70), 4.129 (0.72), 4.148 (1.05), 4.165 (0.67), 6.536 (2.35).

### Intermediate 37

### 5′,6′-dihydrospiro[azetidine-3,4′-pyrrolo[1,2-b]pyrazol]-2′-yl trifluoromethanesulfonate TFA salt

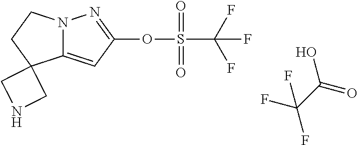

tert-butyl 2′-[(trifluoromethanesulfonyl)oxy]-5′,6′-dihydrospiro[azetidine-3,4′-pyrrolo[1,2-b]pyrazole]-1-carboxylate (1.00 g, 2.52 mmol) was solubilised in dichloromethane (60 mL) under nitrogen, TFA (6.3 mL, 82 mmol) was added and the mixture was stirred for 2 h at rt. The mixture was concentrated under reduced pressure to give 1.23 g of the title compound, which was used without further purification.

LC-MS (method 1): Rt=0.96 min; MS (ESIpos): m/z=298 [M+H]⁺

### Intermediate 38

### 1-(ethylcarbamoyl)-5′,6′-dihydrospiro[azetidine-3,4′-pyrrolo[1,2-b]pyrazol]-2′-yl trifluoromethanesulfonate

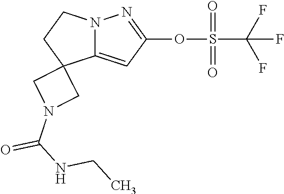 [SMILES: CCNC(=O)N1CC2(CCn3nc(OS(=O)(=O)C(F)(F)F)cc32)C1]

trifluoroacetate-5′,6′-dihydrospiro[azetidine-3,4′-pyrrolo[1,2-b]pyrazol]-2′-yl trifluoromethanesulfonate (1.27 g, 81% purity, 2.50 mmol) was solubilised in dichloromethane (60 mL) under nitrogen, N,N-diisopropylethylamine (4.4 mL, 25 mmol) and isocyanatoethane (990 μL, 13 mmol) were added and the mixture was stirred overnight at rt. The mixture was evaporated and the residue was purified by flash chromatography to give 970 mg of the title compound.

LC-MS (method 1): Rt=0.99 min; MS (ESIpos): m/z=369 [M+H]⁺

¹H-NMR (400 MHz, DMSO-d6) δ[ppm]: 0.984 (6.75), 1.001 (16.00), 1.019 (7.19), 1.035 (0.68), 1.052 (1.23), 1.070 (0.55), 1.137 (1.30), 1.154 (1.27), 2.331 (0.64), 2.518 (3.40), 2.522 (2.19), 2.673 (0.64), 2.771 (2.59), 2.789 (3.93), 2.806 (2.78), 2.986 (0.92), 3.003 (2.90), 3.018 (3.13), 3.022 (3.09), 3.036 (2.93), 3.053 (0.92), 3.934 (1.95), 3.955 (10.37), 3.963 (11.08), 3.983 (2.04), 4.140 (3.03), 4.149 (0.87), 4.158 (4.09), 4.166 (0.83), 4.175 (2.79), 6.443 (0.99), 6.457 (1.98), 6.471 (0.98), 6.498 (10.15).

### Intermediate 39

### (rac)-1-tert-butyl 3-methyl-3-(3-{[tert-butyl(dimethyl)silyl]oxy}propyl)pyrrolidine-1,3-dicarboxylate

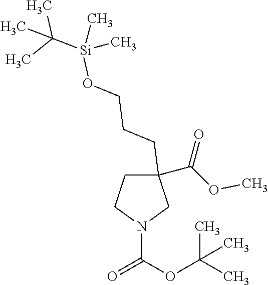 [SMILES: COC(=O)C1(CCCO[Si](C)(C)C(C)(C)C)CCN(C(=O)OC(C)(C)C)C1]

(rac)-1-tert-butyl 3-methyl-pyrrolidine-1,3-dicarboxylate (5.00 g, 21.8 mmol) was solubilised in THF (50 mL), cooled to −78° C. and LiHMDS (44 mL, 1.0 M in THF, 43.6 mmol) was added dropwise. The mixture was stirred at −78° C. for 1 h and (3-bromopropoxy)(tert-butyl)dimethylsilane (10 mL, 44 mmol), solubilised in THF (28 mL), was added dropwise. It was allowed to warm up to rt overnight. The mixture was diluted with sat. NH4Cl solution, extracted with ethyl acetate/THF and the organic layer was dried with a silicone filter and evaporated. The residue was purified by flash chromatography to give 5.65 g (65% yield) of the title compound.

1H-NMR (400 MHz, DMSO-d6): δ [ppm]=−0.04-0.06 (m, 6H), 0.79-0.89 (m, 9H), 1.21-1.35 (m, 2H), 1.38 (s, 9H), 1.60-1.69 (m, 2H), 1.72-1.85 (m, 1H), 2.15-2.26 (m, 1H), 3.08 (dd, 1H), 3.12-3.22 (m, 1H), 3.25-3.31 (m, 1H), 3.53 (t, 2H), 3.60-3.70 (m, 4H).

### Intermediate 40

### (rac)-tert-butyl-3-(3-{[tert-butyl(dimethyl)silyl]oxy}propyl)-3-(3-methoxy-3-oxopropanoyl)pyrrolidine-1-carboxylate

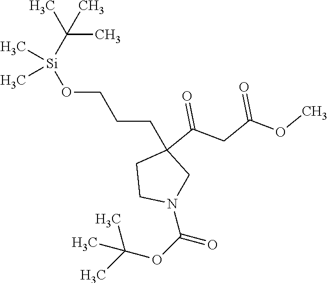

methyl acetate (790 μL, 10 mmol) was solubilised in THF (13 mL), cooled to −78° C. and LiHMDS (10 mL, 1.0 M in THF, 10 mmol) was added dropwise. The mixture was stirred at −78° C. for 1 h and (rac)-1-tert-butyl 3-methyl-3-(3-{[tert-butyl(dimethyl)silyl]oxy}propyl)pyrrolidine-1,3-dicarboxylate (2.00 g, 4.98 mmol), solubilised in THF (2.9 mL), was added dropwise. It was allowed to warm up to rt overnight. The mixture was diluted with sat. NH4Cl solution, extracted with ethyl acetate/THF and the organic layer was dried with a silicone filter and evaporated. The residue was purified by flash chromatography to give 950 mg (43% yield) of the title compound.

1H-NMR (400 MHz, DMSO-d6): δ [ppm]=0.02 (s, 6H), 0.79-0.90 (m, 9H), 1.14-1.31 (m, 2H), 1.38 (br d, 9H), 1.59-1.85 (m, 3H), 2.18-2.25 (m, 1H), 2.99-3.17 (m, 2H), 3.19-3.30 (m, 1H), 3.52 (t, 2H), 3.58-3.86 (m, 6H).

### Intermediate 41

### (rac)-tert-butyl-3-(3-{[tert-butyl(dimethyl)silyl]oxy}propyl)-3-(3-hydroxy-1H-pyrazol-5-yl)pyrrolidine-1-carboxylate

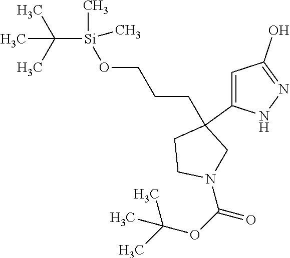 [SMILES: CC(C)(C)OC(=O)N1CCC(CCCO[Si](C)(C)C(C)(C)C)(c2cc(O)n[nH]2)C1]

(rac)-tert-butyl-3-(3-{[tert-butyl(dimethyl)silyl]oxy}propyl)-3-(3-methoxy-3-oxopropanoyl)pyrrolidine-1-carboxylate (950 mg, 2.14 mmol) was solubilised in ethanol (910 μL), hydrazine hydrate (313 μL, 6.42 mmol) was added and the mixture was stirred 1 h at 80° C. It was evaporated and the residue was purified by flash chromatography to give 680 mg (75% yield) of the title compound.

LC-MS (method 1): Rt=1.09 min; MS (ESIpos): m/z=426 [M+H]⁺

1H-NMR (400 MHz, DMSO-d6): δ [ppm]=−0.02 (s, 6H), 0.81-0.86 (m, 9H), 1.10-1.32 (m, 2H), 1.38 (d, 9H), 1.60 (br t, 2H), 1.78-1.92 (m, 1H), 2.10-2.19 (m, 1H), 3.05-3.16 (m, 1H), 3.16-3.23 (m, 1H), 3.25-3.31 (m, 1H), 3.43-3.49 (m, 2H), 3.51-3.59 (m, 1H), 5.28 (s, 1H).

### Intermediate 42

### (rac)-tert-butyl-3-(3-hydroxypropyl)-3-(3-hydroxy-1H-pyrazol-5-yl)pyrrolidine-1-carboxylate

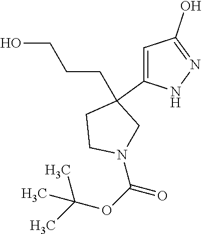 [SMILES: CC(C)(C)OC(=O)N1CCC(CCCO)(c2cc(O)n[nH]2)C1]

(rac)-tert-butyl-3-(3-{[tert-butyl(dimethyl)silyl]oxy}propyl)-3-(3-hydroxy-1H-pyrazol-5-yl)pyrrolidine-1-carboxylate (650 mg, 1.53 mmol) was solubilised in THF (3.3 mL) under nitrogen, cooled to 0° C., TBAF (1.8 mL, 1.0 M in THF, 1.8 mmol) was added and the mixture was stirred for 5 h at 0° C. and was allowed to warm up to rt overnight. It was evaporated and the residue was purified by flash chromatography to give 490 mg of the title compound.

LC-MS (method 1): Rt=0.54 min; MS (ESIpos): m/z=312 [M+H]⁺

1H-NMR (400 MHz, DMSO-d6) δ[ppm]: 0.000 (0.28), 0.849 (0.41), 0.931 (0.76), 0.950 (2.08), 0.968 (0.93), 1.130 (0.31), 1.141 (0.40), 1.150 (0.44), 1.166 (0.50), 1.183 (0.44), 1.201 (0.42), 1.219 (0.56), 1.243 (0.49), 1.255 (0.39), 1.275 (0.24), 1.294 (0.29), 1.313 (0.42), 1.331 (0.43), 1.349 (0.32), 1.384 (16.00), 1.403 (15.99), 1.540 (0.18), 1.586 (1.14), 1.607 (1.50), 1.628 (0.80), 1.642 (0.32), 1.856 (0.44), 1.878 (0.29), 2.154 (0.44), 2.165 (0.42), 2.177 (0.40), 2.535 (1.46), 2.539 (0.95), 3.105 (0.35), 3.113 (0.38), 3.132 (0.67), 3.154 (0.71), 3.161 (0.73), 3.180 (0.90), 3.196 (0.84), 3.272 (1.25), 3.289 (2.31), 3.301 (1.67), 3.571 (0.92), 3.598 (0.82), 4.373 (0.51), 5.288 (0.93), 5.774 (4.98).

### Intermediate 43

### (rac)-tert-butyl-2-hydroxy-6,7-dihydro-5H-spiro[pyrazolo[1,5-a]pyridine-4,3′-pyrrolidine]-1′-carboxylate

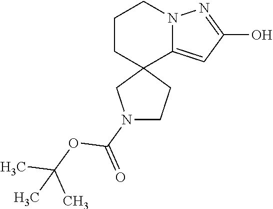 [SMILES: CC(C)(C)OC(=O)N1CCC2(CCCn3nc(O)cc32)C1]

(rac)-tert-butyl-3-(3-hydroxypropyl)-3-(3-hydroxy-1H-pyrazol-5-yl)pyrrolidine-1-carboxylate (490 mg, 1.57 mmol) was solubilised in THF (10 mL), PPh3 (578 mg, 2.20 mmol) was added, DIAD (400 μL, 2.05 mmol) was added dropwise and the mixture was stirred for 2 h at rt. It was evaporated and purified by flash chromatography to give 300 mg (65% yield) of the title compound.

LC-MS (method 1): Rt=0.68 min; MS (ESIpos): m/z=294 [M+H]⁺

1H-NMR (400 MHz, DMSO-d6) δ[ppm]: 1.394 (16.00), 1.414 (15.04), 1.696 (1.15), 1.708 (1.09), 1.727 (0.83), 1.907 (1.01), 1.931 (1.49), 1.944 (1.25), 2.012 (0.51), 2.040 (0.59), 2.337 (0.48), 2.518 (5.97), 2.523 (3.92), 3.262 (0.88), 3.289 (4.19), 3.312 (1.71), 3.364 (1.47), 3.378 (1.09), 3.770 (1.68), 3.785 (2.21), 3.800 (1.07), 3.815 (0.45), 5.246 (2.53), 5.254 (2.37), 5.759 (3.39), 9.471 (4.45).

### Intermediate 44

### (rac)-tert-butyl-2-[(trifluoromethanesulfonyl)oxy]-6,7-dihydro-5H-spiro[pyrazolo[1,5-a]pyridine-4,3′-pyrrolidine]-1′-carboxylate

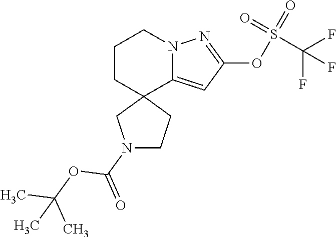 [SMILES: CC(C)(C)OC(=O)N1CCC2(CCCn3nc(OS(=O)(=O)C(F)(F)F)cc32)C1]

(rac)-tert-butyl-2-hydroxy-6,7-dihydro-5H-spiro[pyrazolo[1,5-a]pyridine-4,3′-pyrrolidine]-1′-carboxylate (300 mg, 1.02 mmol), 1,1,1-trifluoro-N-phenyl-N-[(trifluoromethyl)sulfonyl]methanesulfonamide (427 mg, 1.2 mmol) and N,N-diisopropylethylamine (530 μL, 3.1 mmol) were solubilised in THF (6.3 mL) and the mixture was stirred for 3 h at 60° C. It was diluted with brine, extracted 3× with ethyl acetate and the combined organic layers were dried with a silicone filter and evaporated. The residue was purified by preparative HPLC to give 310 mg (71% yield) of the title compound.

LC-MS (method 1): Rt=1.40 min; MS (ESIpos): m/z=426 [M+H]⁺

1H-NMR (400 MHz, DMSO-d6): δ [ppm]=1.40 (d, 9H), 1.71-1.88 (m, 2H), 1.94-2.06 (m, 3H), 2.09-2.19 (m, 1H), 3.35-3.49 (m, 4H), 4.02 (t, 2H), 6.28 (s, 1H).

### Intermediate 45

### (rac)-6,7-dihydro-5H-spiro[pyrazolo[1,5-a]pyridine-4,3′-pyrrolidin]-2-yl trifluoromethanesulfonate

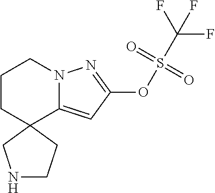 [SMILES: O=S(=O)(Oc1cc2n(n1)CCCC21CCNC1)C(F)(F)F]

(rac)-tert-butyl-2-[(trifluoromethanesulfonyl)oxy]-6,7-dihydro-5H-spiro[pyrazolo[1,5-a]pyridine-4,3′-pyrrolidine]-1′-carboxylate (2.00 g, 4.70 mmol) was solubilised in dichloromethane (80 mL) and TFA (12 mL, 150 mmol) was added. The mixture was stirred for 1 h at rt. The mixture was concentrated under reduced pressure to give 3.20 g of the title compound, which was used without further purification.

LC-MS (method 1): Rt=1.20 min; MS (ESIpos): m/z=326 [M+H]⁺

### Intermediate 46

### (rac)-1′-(ethylcarbamoyl)-6,7-dihydro-5H-spiro[pyrazolo[1,5-a]pyridine-4,3′-pyrrolidin]-2-yl trifluoromethanesulfonate

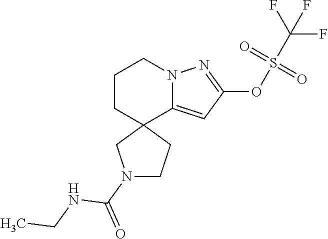 [SMILES: CCNC(=O)N1CCC2(CCCn3nc(OS(=O)(=O)C(F)(F)F)cc32)C1]

(rac)-6,7-dihydro-5H-spiro[pyrazolo[1,5-a]pyridine-4,3′-pyrrolidin]-2-yl trifluoromethanesulfonate (4.20 g, 12.9 mmol) and N,N-diisopropylethylamine (22 mL, 130 mmol) were solubilised in dichloromethane (310 mL), cooled to 0° C., isocyanatoethane (1.0 mL, 13 mmol) was added and the mixture was stirred overnight at rt. It was evaporated and the residue was purified by flash chromatography to give 3.05 g (60% yield) of the title compound.

LC-MS (method 1): Rt=1.08 min; MS (ESIpos): m/z=397 [M+H]⁺

1H-NMR (400 MHz, DMSO-d6) δ[ppm]: 0.902 (0.46), 0.988 (4.24), 1.006 (9.82), 1.024 (4.54), 1.035 (1.52), 1.052 (3.03), 1.070 (1.39), 1.755 (0.65), 1.767 (1.23), 1.773 (1.15), 1.778 (1.14), 1.786 (1.29), 1.797 (0.79), 1.956 (0.41), 1.968 (0.58), 1.974 (0.67), 1.986 (1.27), 1.998 (1.26), 2.005 (1.16), 2.016 (1.24), 2.029 (0.66), 2.035 (0.63), 2.046 (0.47), 2.065 (0.86), 2.099 (0.47), 2.119 (0.96), 2.130 (0.42), 2.138 (0.56), 2.150 (0.66), 2.518 (0.50), 3.006 (0.59), 3.024 (1.87), 3.038 (2.03), 3.041 (2.01), 3.055 (1.82), 3.073 (0.54), 3.322 (1.52), 3.326 (1.39), 3.333 (16.00), 3.347 (3.28), 3.374 (2.59), 3.400 (1.22), 3.405 (0.81), 3.407 (0.74), 3.417 (1.02), 3.422 (1.24), 3.429 (0.70), 3.435 (1.11), 3.440 (0.93), 3.452 (0.94), 3.457 (0.40), 4.018 (1.54), 4.034 (3.27), 4.049 (1.48), 4.344 (0.50), 4.357 (0.97), 4.370 (0.47), 5.758 (12.91), 6.147 (0.68), 6.161 (1.32), 6.174 (0.66), 6.256 (5.96).

### Intermediate 47

### tert-butyl (3'S)-2-bromo-6,7-dihydro-1′H-spiro[pyrazolo[5,1-c][1,4]oxazine-4,3′-pyrrolidine]-1′-carboxylate

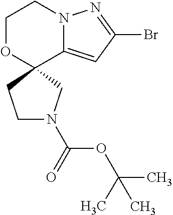 [SMILES: CC(C)(C)OC(=O)N1CC[C@@]2(C1)OCCn1nc(Br)cc12]

The racemate (rac)-tert-butyl-2-bromo-6,7-dihydrospiro[pyrazolo[5,1-c][1,4]oxazine-4,3′-pyrrolidine]-1′-carboxylate (5.00 g, 14.0 mmol) was separated by chiral HPLC to give 1.87 g (96% purity, 37% yield) of the target compound.

Preparative Method:

Instrument: Sepiatec: Prep SFC100; Column: Chiralpak IC 5μ 250×30 mm; eluent A: CO2; eluent B: ethanol+0.2 vol % aqueous ammonia (32%); isocratic: 10% B; flow: 100 mL/min; temperature: 40° C.; BPR: 150 bar; UV: 210 nm

Retention time: 9.5-13.0 min;

Analytical Method:

Instrument: Agilent: 1260, Aurora SFC-Modul; Column: Chiralpak IC 5μ 100×4.6 mm; eluent A: CO2; eluent B: ethanol+0.2 vol % aqueous ammonia (32%); isocratic: 10% B; flow: 4 mL/min; temperature: 37.5° C.; BPR: 100 bar; UV: 210 nm

Retention time: 2.98 min

[α]²⁰_{D}: −29.5° (c=1.00, DMSO)

### Intermediate 48

### tert-butyl (3′R)-2-bromo-6,7-dihydro-1′H-spiro[pyrazolo[5,1-c][1,4]oxazine-4,3′-pyrrolidine]-1′-carboxylate

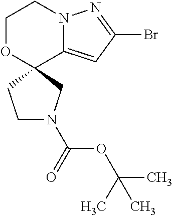 [SMILES: CC(C)(C)OC(=O)N1CC[C@]2(C1)OCCn1nc(Br)cc12]

The racemate (rac)-tert-butyl-2-bromo-6,7-dihydrospiro[pyrazolo[5,1-c][1,4]oxazine-4,3′-pyrrolidine]-1′-carboxylate (5.00 g, 14.0 mmol) was separated by chiral HPLC to give 2.00 g (83% purity, 40% yield) of the target compound.

Preparative Method:

Instrument: Sepiatec: Prep SFC100; Column: Chiralpak IC 5μ 250×30 mm; eluent A: CO2; eluent B: ethanol+0.2 vol % aqueous ammonia (32%); isocratic: 10% B; flow: 100 mL/min; temperature: 40° C.; BPR: 150 bar; UV: 210 nm

Retention time: 15.5-22.0 min;

Analytcal Method:

Instrument: Agilent: 1260, Aurora SFC-Modul; Column: Chiralpak IC 5μ 100×4.6 mm; eluent A: CO2; eluent B: ethanol+0.2 vol % aqueous ammonia (32%); isocratic: 10% B; flow: 4 mL/min; temperature: 37.5° C.; BPR: 100 bar; UV: 210 nm

Retention time: 4.84 min

[α]²⁰_{D}: +27.1° (c=1.00, DMSO)

### Intermediate 49

### (rac)-2-bromo-6,7-dihydrospiro[pyrazolo[5,1-c][1,4]oxazine-4,3′-pyrrolidine]-hydrochloride salt

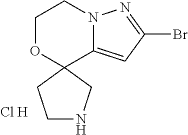

(rac)-tert-butyl-2-bromo-6,7-dihydrospiro[pyrazolo[5,1-c][1,4]oxazine-4,3′-pyrrolidine]-1′-carboxylate (2.00 g, 5.58 mmol) was solubilised in 1,4-dioxane (49 mL) and HCl (28 mL, 4.0 M in 1,4-dioxane, 112 mmol) was added. The mixture was stirred overnight at rt. It was concentrated under reduced pressure to give 1.70 g of the title compound.

LC-MS (method 1): Rt=0.72 min; MS (ESIpos): m/z=258 [M+H]⁺

### Intermediate 50

### (3'S)-2-bromo-6,7-dihydrospiro[pyrazolo[5,1-c][1,4]oxazine-4,3′-pyrrolidine] hydrochloride salt

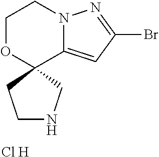

tert-butyl (3'S)-2-bromo-6,7-dihydrospiro[pyrazolo[5,1-c][1,4]oxazine-4,3′-pyrrolidine]-1′-carboxylate (300 mg, 837 μmol) was solubilised in 1,4-dioxane (10 mL) and treated with HCl in dioxane (4.2 mL, 4.0 M, 17 mmol). The reaction mixture was stirred for 18 h at rt and then concentrated under reduced pressure to give 274 mg of the title compound that was used without further purification.

LC-MS (method 1): Rₜ=0.70 min; MS (ESIpos): m/z=260 [M+H]⁺

¹H-NMR (400 MHz, DMSO-d6) δ[ppm]: 2.249 (0.63), 2.260 (0.62), 2.277 (0.50), 2.287 (0.84), 2.311 (0.57), 2.418 (0.60), 2.437 (0.68), 2.453 (0.54), 2.471 (0.67), 2.518 (4.19), 2.523 (2.85), 3.294 (0.94), 3.442 (0.43), 3.468 (0.67), 3.565 (2.74), 3.644 (0.81), 3.674 (0.75), 4.110 (16.00), 6.553 (10.71).

### Intermediate 51

### (3′R)-2-bromo-6,7-dihydrospiro[pyrazolo[5,1-c][1,4]oxazine-4,3′-pyrrolidine]

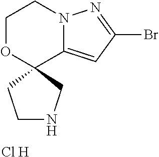

tert-butyl (3′R)-2-bromo-6,7-dihydrospiro[pyrazolo[5,1-c][1,4]oxazine-4,3′-pyrrolidine]-1′-carboxylate (300 mg, 837 μmol) was solubilised in 1,4-dioxane (10 mL) and treated with HCl in dioxane 1,4-dioxane (10 mL). The reaction mixture was stirred at rt for 18 h and concentrated under reduced pressure to give 267 mg of the title compound that was used without further purification

LC-MS (method 1): Rₜ=0.70 min; MS (ESIpos): m/z=260 [M+H]⁺

¹H-NMR (400 MHz, DMSO-d6) δ[ppm]: 2.250 (0.59), 2.260 (0.57), 2.276 (0.45), 2.287 (0.80), 2.311 (0.54), 2.420 (0.56), 2.433 (0.44), 2.438 (0.63), 2.455 (0.47), 2.460 (0.43), 2.518 (3.13), 2.523 (2.12), 3.292 (0.79), 3.309 (0.85), 3.323 (0.99), 3.468 (0.49), 3.565 (3.49), 3.651 (0.53), 3.675 (0.46), 4.111 (16.00), 6.551 (9.85).

### Intermediate 52

### (rac)-2-bromo-N-ethyl-6,7-dihydrospiro[pyrazolo[5,1-c][1,4]oxazine-4,3′-pyrrolidine]-1′-carboxamide

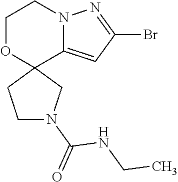 [SMILES: CCNC(=O)N1CCC2(C1)OCCn1nc(Br)cc12]

(rac)-2-bromo-6,7-dihydrospiro[pyrazolo[5,1-c][1,4]oxazine-4,3′-pyrrolidine] (1.70 g, 6.59 mmol) was solubilised in dichloromethane (160 mL), N,N-diisopropylethylamine (11 mL, 66 mmol) was added and the mixture was cooled to 0° C. isocyanatoethane (520 μL, 6.6 mmol) was added and it was stirred overnight under argon at rt. The mixture was evaporated and the residue was purified by flash chromatography to give 2.2 g of the title compound.

LC-MS (method 1): Rt=0.80 min; MS (ESIpos): m/z=329 [M+H]⁺

1H-NMR (400 MHz, DMSO-d6) δ[ppm]: 0.902 (0.84), 0.920 (0.42), 0.990 (4.71), 1.008 (10.94), 1.026 (5.00), 1.035 (8.72), 1.053 (16.00), 1.070 (8.50), 1.138 (0.53), 1.154 (0.52), 2.066 (1.11), 2.184 (0.40), 2.190 (0.56), 2.213 (0.72), 2.216 (0.74), 2.239 (0.53), 2.264 (0.67), 2.281 (0.76), 2.518 (1.19), 2.523 (0.77), 3.004 (0.60), 3.022 (1.97), 3.036 (2.10), 3.039 (2.10), 3.053 (1.94), 3.071 (0.58), 3.275 (0.41), 3.292 (0.60), 3.300 (0.86), 3.316 (0.93), 3.342 (0.66), 3.358 (2.07), 3.386 (2.37), 3.404 (1.21), 3.417 (1.24), 3.422 (4.11), 3.435 (4.20), 3.440 (3.64), 3.452 (3.84), 3.457 (1.41), 3.469 (1.73), 3.488 (1.02), 3.510 (0.50), 3.715 (1.17), 3.741 (0.97), 3.744 (0.98), 4.023 (0.81), 4.040 (1.17), 4.064 (2.93), 4.069 (1.92), 4.075 (4.13), 4.100 (0.83), 4.343 (2.56), 4.355 (5.05), 4.368 (2.49), 5.758 (14.96), 6.150 (0.64), 6.163 (1.27), 6.176 (0.63), 6.465 (9.62).

### Intermediate 53

### (3'S)-2-bromo-N-ethyl-6,7-dihydro-1′H-spiro[pyrazolo[5,1-c][1,4]oxazine-4,3′-pyrrolidine]-1′-carboxamide

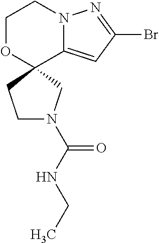 [SMILES: CCNC(=O)N1CC[C@@]2(C1)OCCn1nc(Br)cc12]

N,N-diisopropylethylamine (1.4 mL, 8.3 mmol) was added to a suspension of to a suspension (3'S)-2-bromo-6,7-dihydrospiro[pyrazolo[5,1-c][1,4]oxazine-4,3′-pyrrolidine]hydrochloride salt) (245 mg, 832 μmol) in dichloromethane (10 mL). The reaction mixture was cooled to 0° C. and ethylisocyanate (66 μl, 830 μmol) was added and the reaction was stirred for 18 h. The reaction mixture was then concentrated under reduced pressure and purified by flash column chromatography to give 233 mg (95% purity, 81% yield)

LC-MS (method 1): Rₜ=0.79 min; MS (ESIpos): m/z=331 [M+H]⁺

¹H-NMR (400 MHz, DMSO-d6) δ[ppm]: 0.990 (6.48), 1.008 (16.00), 1.026 (6.98), 2.180 (0.55), 2.184 (0.54), 2.190 (0.78), 2.207 (0.52), 2.213 (0.98), 2.217 (1.01), 2.239 (0.71), 2.265 (0.92), 2.281 (1.04), 2.297 (0.53), 2.314 (0.46), 2.518 (1.42), 2.523 (1.05), 3.004 (0.83), 3.022 (2.70), 3.036 (2.90), 3.039 (2.84), 3.053 (2.65), 3.071 (0.78), 3.159 (4.45), 3.172 (5.03), 3.275 (0.55), 3.292 (0.82), 3.300 (1.16), 3.317 (1.28), 3.343 (0.81), 3.358 (2.84), 3.386 (3.21), 3.468 (0.90), 3.488 (1.36), 3.509 (0.66), 3.712 (1.55), 3.715 (1.55), 3.741 (1.33), 3.744 (1.31), 4.008 (0.53), 4.023 (1.12), 4.040 (1.63), 4.060 (2.63), 4.064 (4.03), 4.069 (2.66), 4.076 (5.70), 4.083 (2.67), 4.095 (1.61), 4.100 (1.16), 4.109 (1.36), 4.122 (0.42), 6.150 (0.88), 6.164 (1.73), 6.177 (0.86), 6.466 (13.96).

### Intermediate 54

### (3′R)-2-bromo-N-ethyl-6,7-dihydro-1′H-spiro[pyrazolo[5,1-c][1,4]oxazine-4,3′-pyrrolidine]-1′-carboxamide

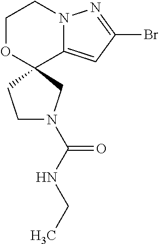 [SMILES: CCNC(=O)N1CC[C@]2(C1)OCCn1nc(Br)cc12]

N,N-diisopropylethylamine (1.4 mL, 8.3 mmol) was added to a suspension of (3′R)-2-bromo-6,7-dihydrospiro[pyrazolo[5,1-c][1,4]oxazine-4,3′-pyrrolidine] hydrochloride salt (enantiomer 2) (245 mg, 832 μmol) in dichloromethane (10 mL) under argon. The reaction was cooled to 0° C. and ethylisocyanate (66 μl, 830 μmol) was added. The reaction mixture was stirred at rt for 18 h, concentrated under reduced pressure and purified by flash column chromatography to give 214 mg (96% purity, 75% yield) of the title compound.

LC-MS (method 1): Rₜ=0.79 min; MS (ESIpos): m/z=331 [M+H]⁺

¹H-NMR (400 MHz, DMSO-d6) δ[ppm]: 0.990 (6.47), 1.008 (16.00), 1.026 (7.01), 2.179 (0.55), 2.184 (0.56), 2.190 (0.77), 2.206 (0.52), 2.213 (0.98), 2.217 (1.02), 2.239 (0.72), 2.264 (0.92), 2.281 (1.05), 2.297 (0.53), 2.314 (0.49), 2.518 (2.18), 2.523 (1.67), 3.004 (0.84), 3.022 (2.73), 3.036 (2.91), 3.039 (2.87), 3.053 (2.68), 3.071 (0.78), 3.159 (3.54), 3.172 (3.71), 3.275 (0.58), 3.292 (0.87), 3.300 (1.18), 3.317 (1.34), 3.342 (0.83), 3.358 (2.86), 3.386 (3.29), 3.467 (0.90), 3.487 (1.36), 3.509 (0.64), 3.712 (1.55), 3.715 (1.55), 3.740 (1.35), 3.744 (1.31), 4.008 (0.54), 4.023 (1.14), 4.040 (1.64), 4.060 (2.64), 4.064 (4.07), 4.069 (2.63), 4.076 (5.69), 4.095 (1.16), 4.100 (1.14), 4.108 (0.93), 6.149 (0.89), 6.163 (1.74), 6.177 (0.85), 6.466 (13.75).

### Intermediate 55

### (3R)-1-{[2-(pyridin-2-yl)propan-2-yl]carbamoyl}-5′,6′-dihydrospiro[pyrrolidine-3,4′-pyrrolo[1,2-b]pyrazol]-2′-yl trifluoromethanesulfonate

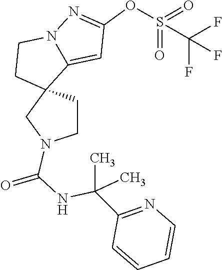 [SMILES: CC(C)(NC(=O)N1CC[C@@]2(CCn3nc(OS(=O)(=O)C(F)(F)F)cc32)C1)c1ccccn1]

A solution of 2-(pyridin-2-yl)propan-2-amine (121 mg, 889 μmol), CDI (157 mg, 970 μmol) and DIPEA (560 μl, 3.2 mmol) in DMF (6 mL) was stirred at RT for 1 h. (3R)-5′,6′-dihydrospiro[pyrrolidine-3,4′-pyrrolo[1,2-b]pyrazol]-2′-yl trifluoromethanesulfonate hydrogen chloride (281 mg, 808 μmol) in DMF (2 mL) was added and the mixture was stirred at RT overnight. The mixture was diluted with water and then extracted with Ethyl acetate 2×. The combined organics were washed with Brine, dried over Sodium sulfate, filtered and concentrated in vacuo to give 484 mg of the title compound.

LC-MS (Method 1): Rₜ=1.19 min; MS (ESIpos): m/z=475 [M+H]⁺

¹H-NMR (400 MHz, DMSO-d6) δ[ppm]: 1.493 (2.66), 1.573 (2.70), 1.578 (2.64), 2.482 (0.67), 2.523 (0.40), 2.728 (13.70), 2.888 (16.00), 3.478 (1.16), 4.241 (0.56), 6.258 (1.58), 6.424 (0.63), 7.436 (0.43), 7.456 (0.48), 7.634 (0.50), 7.951 (2.07), 8.448 (0.43), 8.460 (0.43).

### Intermediate 56

### (3′R)-2-bromo-N-[2-(pyridin-2-yl)propan-2-yl]-6,7-dihydrospiro[pyrazolo[5,1-c][1,4]oxazine-4,3′-pyrrolidine]-1′-carboxamide

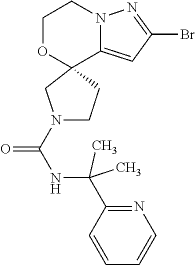 [SMILES: CC(C)(NC(=O)N1CC[C@]2(C1)OCCn1nc(Br)cc12)c1ccccn1]

A solution of 2-(pyridin-2-yl)propan-2-amine (157 mg, 1.15 mmol), CDI (204 mg, 1.26 mmol) and DIPEA (730 μl, 4.2 mmol) in DMF (8 mL) was stirred at RT for 1 h. (3′R)-2-bromo-6,7-dihydrospiro[pyrazolo[5,1-c][1,4]oxazine-4,3′-pyrrolidine] hydrogen chloride (309 mg, 1.05 mmol) in DMF (3 mL) was added and the mixture was stirred at RT overnight. The mixture was diluted with water and then extracted with Ethyl acetate 2×. The combined organics were washed with Brine, dried over Sodium sulfate, filtered and concentrated. The crude was purified by column chromatography to give 282 mg (64% yield) of the title compound.

LC-MS (Method 1): Rₜ=0.98 min; MS (ESIpos): m/z=421 [M+H]⁺

¹H-NMR (400 MHz, DMSO-d6) δ[ppm]: 1.154 (3.10), 1.172 (5.84), 1.190 (3.02), 1.492 (16.00), 1.569 (10.95), 1.576 (10.63), 1.988 (11.74), 2.228 (0.46), 2.253 (0.69), 2.276 (0.58), 2.287 (0.68), 2.303 (0.77), 2.318 (0.92), 2.322 (1.50), 2.327 (1.88), 2.332 (1.49), 2.336 (0.83), 2.518 (6.55), 2.523 (4.43), 2.660 (0.54), 2.664 (1.13), 2.669 (1.63), 2.673 (1.17), 2.678 (0.52), 2.729 (9.88), 2.889 (11.18), 3.357 (0.41), 3.384 (0.60), 3.401 (0.63), 3.438 (0.90), 3.466 (1.03), 3.551 (0.52), 3.570 (0.83), 3.759 (1.25), 3.786 (1.11), 4.000 (0.98), 4.017 (2.83), 4.035 (2.85), 4.053 (1.15), 4.062 (0.72), 4.087 (7.24), 4.114 (0.81), 6.411 (2.62), 6.496 (8.35), 6.504 (0.53), 6.575 (1.73), 7.035 (0.63), 7.136 (0.73), 7.139 (0.81), 7.148 (0.75), 7.151 (0.87), 7.155 (0.85), 7.157 (0.78), 7.167 (0.89), 7.169 (0.91), 7.172 (1.20), 7.175 (1.14), 7.184 (1.05), 7.187 (1.22), 7.191 (1.20), 7.193 (1.15), 7.203 (1.14), 7.206 (1.19), 7.393 (0.76), 7.396 (1.37), 7.399 (0.83), 7.414 (0.93), 7.416 (1.58), 7.419 (0.95), 7.426 (1.16), 7.428 (2.04), 7.431 (1.24), 7.446 (1.30), 7.449 (2.20), 7.648 (0.80), 7.652 (0.89), 7.666 (0.94), 7.670 (0.92), 7.686 (0.65), 7.691 (0.67), 7.701 (1.19), 7.705 (1.14), 7.721 (1.34), 7.725 (1.37), 7.739 (0.93), 7.744 (0.85), 7.951 (1.39), 8.442 (0.89), 8.445 (1.05), 8.448 (1.84), 8.450 (1.95), 8.452 (1.73), 8.454 (2.04), 8.460 (1.85), 8.462 (1.89), 8.464 (1.44), 8.467 (1.17).

### Intermediate 57

### ethyl 2-(3-chloropyridin-4-yl)-2-methylpropanoate

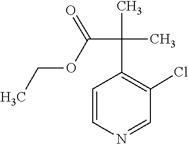

3-Chloropyridine-4-carbonitrile (500 mg, 3.61 mmol) in THF (12 mL) was cooled to −78° C. under nitrogen atmosphere then ethyl 2-methylpropanoate (419 mg, 3.61 mmol) was added and potassium bis(trimethylsilyl)amide (7.9 mL, 0.50 M in toluene, 4.0 mmol) was added dropwise, the mixture was stirred 10 minutes before removing the cooling bath and stirred 1 h at rt. The reaction was quenched with saturated ammonium chloride solution and extracted three times with dichloromethane. The combined organic phases were dried over sodium sulfate and concentrated. The residue was purified by flash chromatography to afford 300 mg (36% yield) of the title compound.

### Intermediate 58

### 2-(3-chloropyridin-4-yl)-2-methylpropanoic acid

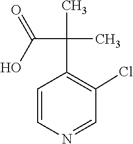

To a stirred solution of ethyl 2-(3-chloropyridin-4-yl)-2-methylpropanoate (300 mg, 1.32 mmol) in methanol (8.5 mL) and THF (26 mL) was added lithium hydroxide (5.3 mL, 1.0 M, 5.3 mmol) and stirred overnight at rt. Lithium hydroxide (2.8 mL, 1.0 M, 2.8 mmol) was added to the mixture and stirred overnight at rt, then the mixture was heated for 4 h at 40° C. and then overnight at 50° C. Potassium hydroxide (1.3 mL, 1.0 M, 1.3 mmol) was added and stirred 4 h at 50° C. and then 72 h at rt. Potassium hydroxide (1.3 mL, 1.0 M, 1.3 mmol) was added again and stirred 2 days at 50° C. The reaction mixture was divided in two portions and stirred in the microwave at 100° C. for 1 h. The reaction mixture was diluted with water, evaporated from organic solvents and extracted three times with dichloromethane. The aqueous phase was acidified to pH 2-3 using hydrochloric acid (9.5 mL, 1M) and extracted with dichloromethane (+10% ethanol). The combined organic phases were concentrated to give 170 mg (64% yield) of the title compound.

### Intermediate 59

### 3-chloro-4-(2-isocyanatopropan-2-yl)pyridine

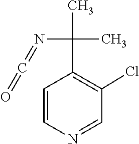

To a stirred solution of 2-(3-chloropyridin-4-yl)-2-methylpropanoic acid (170 mg, 852 μmol) in dioxane (2.8 mL) and triethylamine (150 μL) was added diphenyl phosphorazidate (240 μL, 1.1 mmol) and stirred at rt overnight. The reaction mixture was used in the next step without purification.

### Intermediate 60

### 5-fluoro-2-(2-isocyanatopropan-2-yl)pyridine

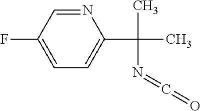

2-(5-fluoropyridin-2-yl)-2-methylpropanoic acid (126 mg, 686 μmol) was solubilised in 1,4-dioxane (2.3 mL), triethylamine (120 μL) and diphenyl phosphorazidate (190 μL, 890 μmol) were added and the mixture was stirred overnight at rt. The reaction mixture was used without further purification.

LC-MS (method 1): Rt=0.75 min; MS (ESIpos): m/z=181 [M+H]⁺

### Intermediate 61

### 1,3-difluoro-5-(2-isocyanatopropan-2-yl)benzene

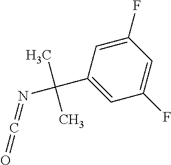

Intermediate 61 was prepared in analogy to Intermediate 60 using 2-(3,5-difluorophenyl)-2-methylpropanoic acid (100 mg, 500 μmol).

### Intermediate 62

### 5-(2-isocyanatopropan-2-yl)-2-methylpyridine

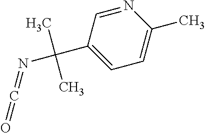

Intermediate 62 was prepared in analogy to Intermediate 60 using 2-methyl-2-(6-methylpyridin-3-yl)propanoic acid (100 mg, 558 μmol).

LC-MS (method 1): Rₜ=1.01 min; MS (ESIpos): m/z=177 [M+H]⁺

### Intermediate 63

### (1-isocyanatocyclohexyl)benzene

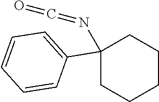 [SMILES: O=C=NC1(c2ccccc2)CCCCC1]

Intermediate 63 was prepared in analogy to Intermediate 60 using 1-phenylcyclohexane-1-carboxylic acid (100 mg, 490 μmol).

### Intermediate 64

### [(1S)-2,2,2-trifluoro-1-isocyanato-1-methoxyethyl]benzene

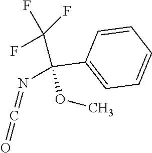 [SMILES: CO[C@@](N=C=O)(c1ccccc1)C(F)(F)F]

Intermediate 64 was prepared in analogy to Intermediate 60 using (2R)-3,3,3-trifluoro-2-methoxy-2-phenylpropanoic acid (120 mg, 512 μmol).

LC-MS (method 1): Rt=0.96 min; MS (ESIpos): m/z=234 [M+H]⁺

### Intermediate 65

### 4-(2-isocyanatopropan-2-yl)pyrimidine

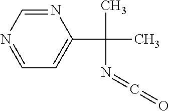

Intermediate 65 was prepared in analogy to Intermediate 60 using 2-methyl-2-(pyrimidin-4-yl)propanoic acid (114 mg, 686 μmol).

LC-MS (method 1): Rt=0.85 min; MS (ESIpos): m/z=164 [M+H]⁺

### Intermediate 66

### 5-(2-isocyanatopropan-2-yl)pyrimidine

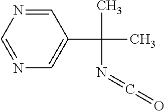

Intermediate 66 was prepared in analogy to Intermediate 60 using 2-methyl-2-(pyrimidin-5-yl)propanoic acid (120 mg, 722 μmol).

LC-MS (method 1): Rt=0.80 min; MS (ESIpos): m/z=164 [M+H]⁺

### Intermediate 67

### 3-isocyanato-3-phenyloxetane

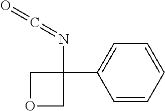 [SMILES: O=C=NC1(c2ccccc2)COC1]

Intermediate 67 was prepared in analogy to Intermediate 60 using 3-phenyloxetane-3-carboxylic acid (100 mg, 561 μmol).

LC-MS (method 1): Rt=1.04 min; MS (ESIpos): m/z=176 [M+H]⁺

### Intermediate 68

### tert-butyl [3-(trifluoroacetyl)pyridin-2-yl]carbamate

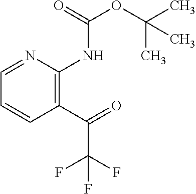 [SMILES: CC(C)(C)OC(=O)Nc1ncccc1C(=O)C(F)(F)F]

tert-butyl pyridin-2-ylcarbamate (3.00 g, 15.4 mmol) and N,N,N′,N′-Tetramethylethylenediamine (5.8 mL, 39 mmol) were solubilised in THF (150 mL) under argon, cooled to −78° C., BuLi (24 mL, 1.6 M, 39 mmol) was added dropwise and the mixture was stirred for 30 min at 0° C. The mixture was cooled to −78° C., ethyl trifluoroacetate (2.5 mL, 23 mmol) was added and it was stirred for 1 h at 0° C. It was diluted with sat. NH4Cl solution and extracted 3× with ethyl acetate. The combined organic layers were dried and evaporated. The residue was stirred in hexane, filtered and the solid was dried under reduced pressure at 40° C. to give 2.40 g (54% yield) of the title compound.

LC-MS (method 1): Rₜ=0.64 min; MS (ESIneg): m/z=289 [M−H]⁻

¹H-NMR (400 MHz, DMSO-d6) δ[ppm]: 1.406 (0.41), 1.426 (16.00), 1.433 (2.41), 1.466 (0.56), 1.494 (0.53), 2.445 (0.50), 7.289 (0.63), 7.301 (0.65), 7.308 (0.70), 7.320 (0.70), 7.921 (0.49), 7.940 (0.43), 8.546 (0.68), 8.551 (0.72), 8.558 (0.71), 8.563 (0.68), 10.635 (0.62).

### Intermediate 69

### 1-(2-aminopyridin-3-yl)-2,2,2-trifluoroethan-1-one-hydrogen chloride (1/1)

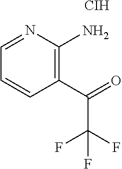

tert-butyl [3-(trifluoroacetyl)pyridin-2-yl]carbamate (2.40 g, 80% purity, 6.62 mmol) was solubilised in 1,4-dioxane (25 mL), HCl (17 mL, 4.0 M in dioxane, 66 mmol) was added and the mixture was stirred over night at rt. It was concentrated under reduced pressure to give 1.37 g (91% yield) of the title compound, which was used without further purification.

LC-MS (method 1): Rₜ=0.84 min; MS (ESIneg): m/z=189 [M−H]⁻

¹H-NMR (400 MHz, DMSO-d6) δ[ppm]: 0.884 (0.98), 0.899 (1.36), 0.917 (2.66), 0.936 (1.13), 1.154 (0.69), 1.172 (1.38), 1.190 (0.81), 1.325 (0.53), 1.343 (0.61), 1.362 (0.56), 1.499 (8.43), 1.517 (0.87), 1.709 (0.45), 1.987 (2.50), 2.336 (1.33), 2.518 (16.00), 2.523 (10.31), 2.678 (1.41), 2.831 (10.97), 3.483 (3.32), 3.565 (3.19), 3.677 (0.42), 4.017 (0.59), 4.034 (0.59), 6.741 (14.18), 6.752 (13.60), 6.761 (13.96), 6.772 (14.85), 6.946 (3.11), 6.961 (4.04), 6.964 (3.88), 6.980 (3.58), 6.994 (0.63), 7.307 (0.53), 7.328 (0.52), 7.407 (0.40), 7.649 (1.47), 7.924 (0.48), 7.940 (0.53), 8.021 (1.68), 8.026 (4.85), 8.031 (7.30), 8.037 (5.67), 8.041 (3.61), 8.047 (6.17), 8.052 (9.02), 8.057 (8.79), 8.068 (7.57), 8.090 (5.72), 8.095 (7.38), 8.106 (5.18), 8.110 (6.30), 8.116 (4.14), 8.135 (3.43), 8.408 (10.48), 8.413 (11.25), 8.419 (11.04), 8.424 (10.49), 8.536 (0.70), 8.550 (0.64), 8.610 (9.61), 8.758 (0.43).

### Intermediate 70

### (rac)-2-nitro-3-{[1-phenylethyl]sulfanyl}pyridine

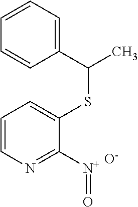

3-fluoro-2-nitropyridine (50.0 mg, 352 μmol), (rac)-1-phenylethane-1-thiol (50.1 mg, 362 μmol) and Cs2CO3 (126 mg, 387 μmol) were stirred in DMSO (500 μL) for 1 h at 65° C. The mixture was diluted with water and filtered. The precipitate was washed with water and dried under reduced pressure at 50° C. to give 61.0 mg (86% purity, 57% yield) of the title compound, which was used without further purification.

¹H-NMR (400 MHz, DMSO-d6): δ [ppm]=1.59 (d, 3H), 4.92 (q, 1H), 7.21-7.27 (m, 1H), 7.29-7.34 (m, 2H), 7.41-7.46 (m, 2H), 7.71 (dd, 1H), 8.29 (dd, 1H), 8.36 (dd, 1H).

LC-MS (method 1): Rt=1.27 min; MS (ESIpos): m/z=261 [M+H]⁺

### Intermediate 71

### 5-bromo-3-[(cyclohexylmethyl)sulfanyl]-2-nitropyridine

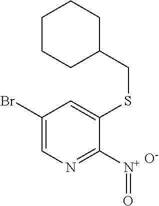

5-bromo-3-fluoro-2-nitropyridine (800 mg, 3.62 mmol), cyclohexylmethanethiol (486 mg, 3.73 mmol) and Na2CO3 (422 mg, 3.98 mmol) were stirred in DMSO (5.1 mL) for 1 h at 65° C. The mixture was diluted with water, stirred for 30 min at rt and extracted 2× with DCM. The combined organic layers were washed with water, dried over a silicone filter and concentrated under reduced pressure to give 1.32 g (84% purity, 92% yield) of the title compound, which was used without further purification.

¹H NMR (400 MHz, DMSO-d6) δ ppm 0.97-1.28 (m, 8H) 1.49-1.72 (m, 6H) 1.75-1.85 (m, 3H) 3.06 (d, 2H) 8.39 (d, 1H) 8.52 (d, 1H)

LC-MS (method 1): Rt=1.56 min; MS (ESIpos): m/z=331 [M+H]⁺

### Intermediate 72

### 3-(benzylsulfanyl)-5-bromo-2-nitropyridine

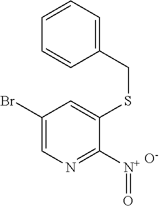

5-bromo-3-fluoro-2-nitropyridine (100 mg, 453 μmol), phenylmethanethiol (57.9 mg, 466 μmol) and CsHCO3 (96.5 mg, 498 μmol) were stirred in DMSO (640 μL) for 2 h at 65° C. The mixture was diluted with water and filtered. The precipitate was washed with water and dried under reduced pressure at 50° C. to give 110 mg (93% purity, 70% yield) of the title compound, which was used without further purification.

¹H-NMR (500 MHz, DMSO-d6): δ [ppm]=4.49 (s, 2H), 7.27-7.32 (m, 1H), 7.33-7.38 (m, 2H), 7.41-7.45 (m, 2H), 8.50 (d, 1H), 8.54 (d, 1H).

LC-MS (method 1): Rt=1.35 min; MS (ESIpos): m/z=325 [M+H]⁺

### Intermediate 73

### (rac)-5-bromo-3-{[1-cyclohexylethyl]sulfanyl}-2-nitropyridine

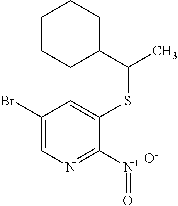

Intermediate 73 was prepared in analogy to Intermediate 71 using 5-bromo-3-fluoro-2-nitropyridine (300 mg, 1.36 mmol) and (rac)-1-cyclohexylethane-1-thiol (202 mg, 1.40 mmol).

LC-MS (method 1): Rt=1.62 min; MS (ESIpos): m/z=345 [M+H]⁺

¹H-NMR (400 MHz, DMSO-d6) δ[ppm]: 1.091 (0.42), 1.107 (0.42), 1.121 (0.47), 1.129 (0.59), 1.157 (0.71), 1.189 (0.48), 1.202 (0.45), 1.206 (0.44), 1.215 (3.06), 1.232 (3.03), 1.302 (0.46), 1.319 (0.46), 1.705 (0.58), 1.729 (0.60), 2.518 (0.45), 2.540 (16.00), 8.528 (0.67), 8.533 (1.74), 8.538 (2.17), 8.543 (0.89).

### Intermediate 74

### 5-bromo-3-[(cyclopentylmethyl)sulfanyl]-2-nitropyridine

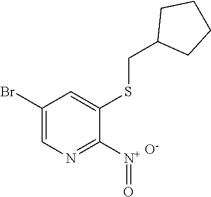

Intermediate 74 was prepared in analogy to Intermediate 71 using 5-bromo-3-fluoro-2-nitropyridine (500 mg, 2.26 mmol) and cyclopentylmethanethiol (271 mg, 2.33 mmol).

LC-MS (method 1): Rt=1.49 min; MS (ESIpos): m/z=317 [M+H]⁺

1H-NMR (400 MHz, DMSO-d6) δ[ppm]: 1.176 (0.83), 1.193 (1.95), 1.207 (2.14), 1.211 (2.35), 1.223 (2.93), 1.225 (2.97), 1.228 (3.23), 1.242 (2.67), 1.255 (2.21), 1.266 (1.60), 1.275 (2.94), 1.285 (2.78), 1.293 (3.28), 1.305 (3.58), 1.324 (3.05), 1.342 (1.37), 1.453 (0.57), 1.473 (1.59), 1.483 (2.72), 1.491 (3.54), 1.492 (3.60), 1.502 (5.52), 1.510 (5.66), 1.521 (6.97), 1.529 (4.30), 1.541 (4.72), 1.549 (3.20), 1.563 (4.25), 1.568 (3.28), 1.574 (2.74), 1.579 (3.64), 1.584 (3.80), 1.592 (3.27), 1.601 (3.66), 1.608 (4.54), 1.613 (3.40), 1.625 (4.47), 1.637 (3.28), 1.646 (2.22), 1.665 (0.79), 1.675 (0.50), 1.716 (1.14), 1.722 (1.11), 1.725 (1.32), 1.732 (2.17), 1.735 (1.94), 1.746 (3.20), 1.749 (2.70), 1.765 (3.03), 1.776 (3.40), 1.794 (3.54), 1.807 (3.59), 1.826 (3.07), 1.838 (2.22), 1.854 (1.04), 2.041 (1.16), 2.060 (2.64), 2.079 (3.47), 2.098 (2.96), 2.119 (3.39), 2.139 (3.87), 2.157 (2.77), 2.176 (1.16), 2.518 (2.48), 2.523 (1.61), 2.539 (13.08), 2.710 (16.00), 2.729 (15.06), 3.116 (1.92), 3.134 (2.13), 3.143 (11.40), 3.161 (11.09), 3.194 (2.76), 3.212 (2.81), 3.221 (2.23), 3.239 (2.15), 3.245 (0.65), 3.264 (0.50), 3.572 (0.40), 7.853 (1.18), 7.858 (1.22), 8.056 (0.93), 8.061 (0.91), 8.079 (0.91), 8.085 (0.96), 8.234 (0.43), 8.240 (0.42), 8.251 (1.64), 8.256 (1.64), 8.405 (7.33), 8.410 (8.22), 8.492 (1.01), 8.494 (1.13), 8.496 (1.15), 8.499 (1.00), 8.519 (9.61), 8.523 (8.30).

### Intermediate 75

### 5-bromo-3-(methylsulfanyl)pyridin-2-amine

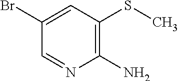

5-bromo-3-iodopyridin-2-amine (2.00 g, 6.69 mmol), sodium methanethiolate (2.81 g, 40.14 mmol) CuI (762 mg, 4.01 mmol) and ethylene glycol (660 μL, 12.06 mmol) were stirred in 2-propanol (16 mL) at 100° C. for 3 d. The mixture was evaporated and the residue was purified by flash chromatography to give 680 mg (46% yield) of the title compound.

LC-MS (method 1): Rt=0.99 min; MS (ESIpos): m/z=219 [M+H]⁺

¹H-NMR (400 MHz, DMSO-d₆): δ [ppm]=2.42 (s, 3H), 6.13 (s, 2H), 7.53 (d, 1H), 7.86 (d, 1H).

### Intermediate 76

### 5-bromo-3-(phenylsulfanyl)pyridin-2-amine

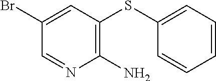 [SMILES: Nc1ncc(Br)cc1Sc1ccccc1]

Intermediate 76 was prepared in analogy to Intermediate 75 using 5-bromo-3-iodopyridin-2-amine (2.00 g, 6.69 mmol) and sodium benzenethiolate (1.77 g, 13.4 mmol).

LC-MS (method 1): Rₜ=1.29 min; MS (ESIpos): m/z=281 [M+H]⁺

1H-NMR (400 MHz, DMSO-d6) δ[ppm]: 2.021 (0.98), 2.518 (0.94), 2.522 (0.61), 4.188 (0.57), 6.423 (11.24), 7.188 (1.18), 7.193 (8.74), 7.196 (11.55), 7.209 (3.30), 7.214 (16.00), 7.217 (12.99), 7.225 (3.34), 7.228 (1.85), 7.238 (2.46), 7.243 (7.65), 7.248 (2.13), 7.258 (3.91), 7.261 (5.77), 7.265 (2.69), 7.317 (2.11), 7.322 (11.43), 7.326 (4.45), 7.339 (7.78), 7.342 (14.16), 7.346 (3.36), 7.356 (2.37), 7.360 (5.68), 7.365 (1.20), 7.769 (13.05), 7.774 (13.64), 7.967 (0.49), 8.014 (0.52), 8.019 (0.44), 8.090 (14.79), 8.096 (14.91).

### Intermediate 77

### 5-bromo-2-nitro-3-[(1R)-1-(pyridin-3-yl)ethoxy]pyridine

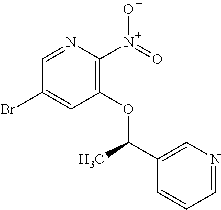 [SMILES: C[C@@H](Oc1cc(Br)cnc1[N+](=O)[O-])c1cccnc1]

5-bromo-2-nitropyridin-3-ol (150 mg, 685 μmol), (1S)-1-(pyridin-3-yl)ethan-1-ol (84.4 mg, 685 μmol) and PPh3 (234 mg, 890 μmol) were stirred in THF (6.0 mL) for 1 h at rt. The mixture was cooled to 0° C., DIAD (180 μL, 890 μmol) was added and the mixture was stirred overnight at rt. It was evaporated and purified by preparative HPLC to give 70.0 mg (92% purity, 29% yield) of the title compound.

1H-NMR (400 MHz, DMSO-d6): δ [ppm]=1.62 (d, 3H), 6.05 (q, 1H), 7.44 (ddd, 1H), 7.83 (dt, 1H), 8.26 (d, 1H), 8.36 (d, 1H), 8.54 (dd, 1H), 8.67 (d, 1H).

LC-MS (method 2): Rt=0.89 min; MS (ESIpos): m/z=326 [M+H]⁺

### Intermediate 78

### 5-bromo-2-nitro-3-[(1S)-1-(pyridin-3-yl)ethoxy]pyridine

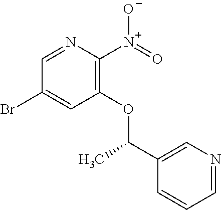 [SMILES: C[C@H](Oc1cc(Br)cnc1[N+](=O)[O-])c1cccnc1]

Intermediate 78 was prepared in analogy to Intermediate 77 using 5-bromo-2-nitropyridin-3-ol (250 mg, 1.14 mmol) and (1R)-1-(pyridin-3-yl)ethan-1-ol (155 mg, 1.26 mmol).

LC-MS (method 1): Rt=0.92 min; MS (ESIpos): m/z=326 [M+H]⁺

### Intermediate 79

### 5-bromo-2-nitro-3-[(1R)-1-(pyridin-4-yl)ethoxy]pyridine

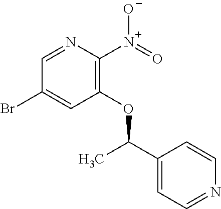 [SMILES: C[C@@H](Oc1cc(Br)cnc1[N+](=O)[O-])c1ccncc1]

Intermediate 79 was prepared in analogy to Intermediate 77 using 5-bromo-2-nitropyridin-3-ol (75.0 mg, 342 μmol) and (1R)-1-(pyridin-4-yl)ethan-1-ol (42.2 mg, 342 μmol).

LC-MS (method 2): Rt=0.88 min; MS (ESIpos): m/z=326 [M+H]⁺

### Intermediate 80

### 5-bromo-2-nitro-3-[(1S)-1-(pyridin-4-yl)ethoxy]pyridine

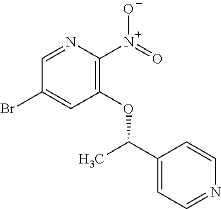 [SMILES: C[C@H](Oc1cc(Br)cnc1[N+](=O)[O-])c1ccncc1]

Intermediate 80 was prepared in analogy to Intermediate 77 using 5-bromo-2-nitropyridin-3-ol (500 mg, 2.28 mmol) and (1S)-1-(pyridin-4-yl)ethan-1-ol (281 mg, 2.28 mmol).

LC-MS (method 2): Rt=0.87 min; MS (ESIpos): m/z=326 [M+H]⁺

### Intermediate 81

### (rac)-5-{1-[(5-bromo-2-nitropyridin-3-yl)oxy]ethyl}pyrimidine

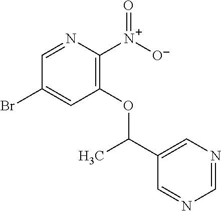 [SMILES: CC(Oc1cc(Br)cnc1[N+](=O)[O-])c1cncnc1]

Intermediate 81 was prepared in analogy to Intermediate 77 using 5-bromo-2-nitropyridin-3-ol (826 mg, 3.77 mmol) and (rac)-1-(pyrimidin-5-yl)ethan-1-ol (468 mg, 3.77 mmol).

LC-MS (method 1): Rt=0.97 min; MS (ESIpos): m/z=327 [M+H]⁺

¹H-NMR (400 MHz, DMSO-d₆): δ [ppm]=1.67 (d, 3H), 6.07 (q, 1H), 8.30 (d, 1H), 8.44 (d, 1H), 8.90 (s, 2H), 9.18 (s, 1H).

### Intermediate 82

### 3-[(2,6-dichlorophenyl)methoxy]-2-nitropyridine

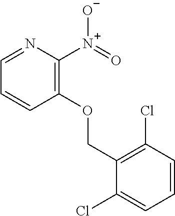 [SMILES: O=[N+]([O-])c1ncccc1OCc1c(Cl)cccc1Cl]

Intermediate 82 was prepared in analogy to Intermediate 77 using (2,6-dichlorophenyl)methanol (500 mg, 2.82 mmol) and 2-nitropyridin-3-ol (435 mg, 3.11 mmol).

LC-MS (method 1): Rt=1.25 min; MS (ESIpos): m/z=299 [M+H]⁺

¹H-NMR (400 MHz, DMSO-d₆): δ [ppm]=5.49 (s, 2H), 7.47-7.54 (m, 1H), 7.55-7.62 (m, 2H), 7.85 (dd, 1H), 8.19 (dd, 1H), 8.25 (dd, 1H).

### Intermediate 83

### 3-[(2-fluorophenyl)methoxy]-2-nitropyridine

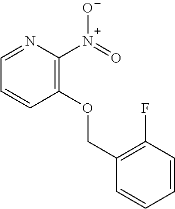 [SMILES: O=[N+]([O-])c1ncccc1OCc1ccccc1F]

Intermediate 83 was prepared in analogy to Intermediate 77 using (2-fluorophenyl)methanol (430 mg, 3.41 mmol) and 2-nitropyridin-3-ol (478 mg, 3.41 mmol).

LC-MS (method 1): Rt=1.15 min; MS (ESIpos): m/z=249 [M+H]⁺

¹H-NMR (400 MHz, DMSO-d₆): δ [ppm]=5.42 (s, 2H), 7.23-7.32 (m, 2H), 7.42-7.49 (m, 1H), 7.54 (td, 1H), 7.80 (dd, 1H), 8.11-8.16 (m, 2H).

### Intermediate 84

### 3-[(3-fluorophenyl)methoxy]-2-nitropyridine

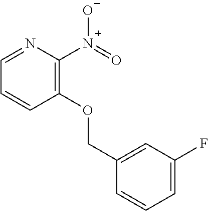 [SMILES: O=[N+]([O-])c1ncccc1OCc1cccc(F)c1]

Intermediate 84 was prepared in analogy to Intermediate 77 using (3-fluorophenyl)methanol (430 mg, 3.41 mmol) and 2-nitropyridin-3-ol (478 mg, 3.41 mmol).

LC-MS (method 1): Rt=1.16 min; MS (ESIpos): m/z=249 [M+H]⁺

¹H-NMR (400 MHz, DMSO-d₆): δ [ppm]=5.40 (s, 2H), 7.17-7.23 (m, 1H), 7.24-7.30 (m, 2H), 7.47 (td, 1H), 7.79 (dd, 1H), 8.04 (dd, 1H), 8.14 (dd, 1H).

### Intermediate 85

### 3-[(2-chlorophenyl)methoxy]-2-nitropyridine

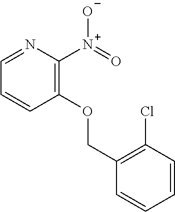 [SMILES: O=[N+]([O-])c1ncccc1OCc1ccccc1Cl]

Intermediate 85 was prepared in analogy to Intermediate 77 using (2-chlorophenyl)methanol (450 mg, 3.16 mmol) and 2-nitropyridin-3-ol (486 mg, 3.47 mmol).

LC-MS (method 1): Rt=1.23 min; MS (ESIpos): m/z=265 [M+H]⁺

¹H-NMR (400 MHz, DMSO-d₆): δ [ppm]=5.42 (s, 2H), 7.41-7.44 (m, 2H), 7.52-7.55 (m, 1H), 7.57-7.61 (m, 1H), 7.81 (dd, 1H), 8.12 (dd, 1H), 8.16 (dd, 1H).

### Intermediate 86

### 2-nitro-3-[(pyridin-2-yl)methoxy]pyridine

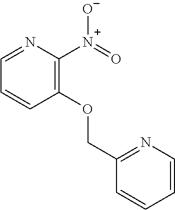 [SMILES: O=[N+]([O-])c1ncccc1OCc1ccccn1]

Intermediate 86 was prepared in analogy to Intermediate 77 using (pyridin-2-yl)methanol (400 mg, 3.67 mmol) and 2-nitropyridin-3-ol (514 mg, 3.67 mmol).

LC-MS (method 1): Rt=0.90 min; MS (ESIpos): m/z=233 [M+H]⁺

¹H-NMR (400 MHz, DMSO-d₆): δ [ppm]=5.45 (s, 2H), 7.38 (ddd, 1H), 7.49 (d, 1H), 7.78 (dd, 1H), 7.88 (td, 1H), 8.07 (dd, 1H), 8.14 (dd, 1H), 8.56-8.61 (m, 1H).

### Intermediate 87

### 3-[(2-chloro-5-fluorophenyl)methoxy]-2-nitropyridine

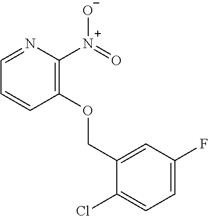 [SMILES: O=[N+]([O-])c1ncccc1OCc1cc(F)ccc1Cl]

Intermediate 87 was prepared in analogy to Intermediate 77 using (2-chloro-5-fluorophenyl)methanol (589 mg, 3.67 mmol) and 2-nitropyridin-3-ol (514 mg, 3.67 mmol).

LC-MS (method 1): Rt=1.24 min; MS (ESIpos): m/z=283 [M+H]⁺

¹H-NMR (400 MHz, DMSO-d₆): δ [ppm]=5.41 (s, 2H), 7.32 (td, 1H), 7.45 (dd, 1H), 7.60 (dd, 1H), 7.82 (dd, 1H), 8.13 (dd, 1H), 8.18 (dd, 1H).

### Intermediate 88

### 5-bromo-2-nitro-3-[(1S)-1-(pyridin-2-yl)ethoxy]pyridine

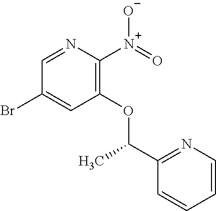 [SMILES: C[C@H](Oc1cc(Br)cnc1[N+](=O)[O-])c1ccccn1]

Intermediate 88 was prepared in analogy to Intermediate 77 using 5-bromo-2-nitropyridin-3-ol (889 mg, 4.06 mmol) and (1R)-1-(pyridin-2-yl)ethan-1-ol (500 mg, 4.06 mmol).

LC-MS (method 1): Rₜ=1.17 min; MS (ESIpos): m/z=326 [M+H]⁺

¹H-NMR (400 MHz, DMSO-d6) δ[ppm]: 1.133 (1.06), 1.153 (1.88), 1.170 (16.00), 1.185 (15.55), 1.615 (2.04), 1.631 (2.09), 1.986 (1.73), 2.518 (0.46), 4.734 (0.44), 4.750 (1.09), 4.766 (1.44), 4.781 (1.08), 4.797 (0.44), 5.933 (0.43), 5.949 (0.43), 7.476 (0.48), 7.496 (0.54), 7.859 (0.47), 7.864 (0.48), 8.241 (0.69), 8.245 (1.05), 8.259 (1.49), 8.264 (0.93), 8.882 (3.03).

### Intermediate 89

### 5-bromo-3-[(pyridazin-3-yl)methoxy]pyridin-2-amine

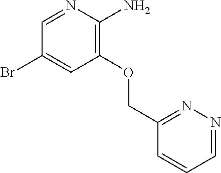 [SMILES: Nc1ncc(Br)cc1OCc1cccnn1]

Intermediate 89 was prepared in analogy to Intermediate 77 using 2-amino-5-bromopyridin-3-ol (100 mg, 529 μmol) and (pyridazin-3-yl)methanol (58.3 mg, 529 μmol).

LC-MS (method 1): Rt=0.74 min; MS (ESIpos): m/z=283 [M+H]⁺

¹H-NMR (400 MHz, DMSO-d₆): δ [ppm]=5.44 (s, 2H), 6.12 (s, 2H), 7.40 (d, 1H), 7.63 (d, 1H), 7.77 (dd, 1H), 8.01 (dd, 1H), 9.22 (dd, 1H).

### Intermediate 90

### (rac)-2-{1-[(5-bromo-2-nitropyridin-3-yl)oxy]ethyl}-5-chloro-3-fluoropyridine

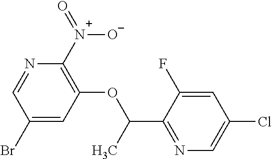 [SMILES: CC(Oc1cc(Br)cnc1[N+](=O)[O-])c1ncc(Cl)cc1F]

Intermediate 90 was prepared in analogy to Intermediate 77 using 5-bromo-2-nitropyridin-3-ol (200 mg, 913 μmol) and (rac)-1-(5-chloro-3-fluoropyridin-2-yl)ethan-1-ol (160 mg, 913 μmol).

LC-MS (method 1): Rt=1.33 min; MS (ESIpos): m/z=375 [M+H]⁺

¹H-NMR (400 MHz, DMSO-d₆): δ [ppm]=1.67 (d, 3H), 6.17 (q, 1H), 8.19 (dd, 1H), 8.29 (d, 1H), 8.36 (d, 1H), 8.54 (dd, 1H).

### Intermediate 91

### 5-bromo-3-[(1R)-1-(2-chlorophenyl)ethoxy]-2-nitropyridine

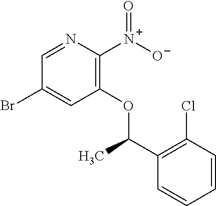 [SMILES: C[C@@H](Oc1cc(Br)cnc1[N+](=O)[O-])c1ccccc1Cl]

(1R)-1-(2-chlorophenyl)ethan-1-ol (521 mg, 3.33 mmol) was solubilised in THF (32 mL) under argon, cooled to 0° C., NaH (380 mg, 60% purity, 9.50 mmol) was added and the mixture was stirred for 30 min at 0° C. 5-bromo-3-fluoro-2-nitropyridine (700 mg, 3.17 mmol) was added and it was stirred for 1 h at 0° C. The mixture was diluted with sat. NH4Cl solution, extracted 2× with ethyl acetate and the combined organic layers were washed with brine, dried over Na2SO4 and concentrated under reduced pressure to give 1.54 g of the title compound, which was used without further purification.

LC-MS (method 1): Rₜ=1.44 min; MS (ESIpos): m/z=357 [M+H]⁺

### Intermediate 92

### 5-bromo-3-[(1S)-1-(2-chlorophenyl)ethoxy]-2-nitropyridine

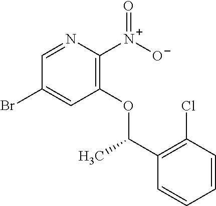 [SMILES: C[C@H](Oc1cc(Br)cnc1[N+](=O)[O-])c1ccccc1Cl]

Intermediate 92 was prepared in analogy to Intermediate 91 using 5-bromo-3-fluoro-2-nitropyridine (700 mg, 3.17 mmol) and (1S)-1-(2-chlorophenyl)ethan-1-ol (521 mg, 3.33 mmol).

LC-MS (method 1): Rₜ=1.44 min; MS (ESIpos): m/z=357 [M+H]⁺

### Intermediate 93

### 5-bromo-2-nitro-3-[(1R)-1-(pyridin-2-yl)ethoxy]pyridine

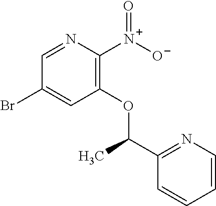 [SMILES: C[C@@H](Oc1cc(Br)cnc1[N+](=O)[O-])c1ccccn1]

Intermediate 93 was prepared in analogy to Intermediate 91 using 5-bromo-3-fluoro-2-nitropyridine (300 mg, 1.36 mmol) and (1R)-1-(pyridin-2-yl)ethan-1-ol (176 mg, 1.43 mmol).

LC-MS (method 1): Rₜ=1.14 min; MS (ESIpos): m/z=326 [M+H]⁺

### Intermediate 94

### 5-bromo-3-[(1S)-1-(pyridin-2-yl)ethoxy]pyridin-2-amine

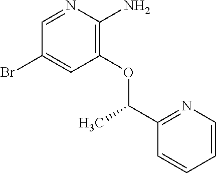 [SMILES: C[C@H](Oc1cc(Br)cnc1N)c1ccccn1]

Intermediate 94 was prepared in analogy to Intermediate 91 using 5-bromo-3-fluoropyridin-2-amine (500 mg, 2.62 mmol) and (1S)-1-(pyridin-2-yl)ethan-1-ol (339 mg, 2.75 mmol).

¹H-NMR (400 MHz, DMSO-d6): δ [ppm]=1.60 (d, 3H), 5.51 (q, 1H), 6.06 (s, 2H), 7.02 (d, 1H), 7.31 (ddd, 1H), 7.47-7.57 (m, 2H), 7.81 (td, 1H), 8.52-8.59 (m, 1H).

LC-MS (method 1): Rₜ=0.95 min; MS (ESIpos): m/z=294 [M+H]⁺

¹H-NMR (400 MHz, DMSO-d6) δ[ppm]: 1.342 (1.35), 1.358 (1.37), 1.591 (15.49), 1.598 (2.65), 1.606 (16.00), 1.616 (1.52), 2.420 (0.79), 2.440 (0.48), 2.518 (2.24), 2.523 (1.60), 2.824 (0.45), 2.836 (0.44), 2.995 (2.46), 5.332 (0.50), 5.344 (0.43), 5.482 (0.95), 5.497 (3.35), 5.514 (3.36), 5.530 (1.00), 5.758 (1.85), 6.065 (6.33), 6.471 (0.63), 7.014 (5.76), 7.019 (5.80), 7.297 (2.15), 7.300 (2.22), 7.309 (2.22), 7.312 (2.35), 7.316 (2.38), 7.319 (2.40), 7.328 (2.34), 7.331 (2.41), 7.502 (4.02), 7.521 (12.34), 7.526 (9.41), 7.572 (1.12), 7.664 (0.44), 7.669 (0.46), 7.691 (0.44), 7.696 (0.46), 7.786 (2.25), 7.791 (2.32), 7.806 (3.41), 7.810 (3.49), 7.825 (1.82), 7.829 (1.82), 7.844 (0.61), 7.846 (0.64), 7.849 (0.59), 7.851 (0.56), 8.544 (2.33), 8.546 (2.83), 8.548 (2.81), 8.551 (2.63), 8.556 (2.46), 8.558 (2.91), 8.561 (2.65), 8.563 (2.45).

### Intermediate 95

### 5-bromo-3-[(3-methylphenyl)methoxy]pyridin-2-amine

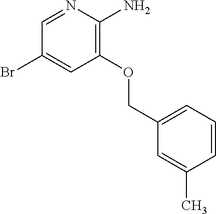 [SMILES: Cc1cccc(COc2cc(Br)cnc2N)c1]

(3-methylphenyl)methanol (20.1 mg, 165 μmol) was solubilised in DMSO (2 mL) under argon, NaH (219 mg, 60% purity, 314 μmol) was added and the mixture was stirred for 1 h at rt. 5-bromo-3-fluoropyridin-2-amine (30.0 mg, 157 μmol) was added and it was stirred for 20 h at 100° C. The mixture was diluted with water, extracted 3× with DCM and the combined organic layers were dried over MgSO₄, filtered and concentrated under reduced pressure. The residue was purified by flash chromatography to give 30 mg (65% yield) of the title compound

LC-MS (method 1): Rₜ=1.27 min; MS (ESIpos): m/z=295 [M+H]⁺

¹H-NMR (400 MHz, DMSO-d6) δ[ppm]: 1.156 (1.67), 1.165 (1.40), 1.233 (0.97), 2.251 (0.45), 2.289 (1.46), 2.323 (16.00), 2.331 (1.33), 2.336 (0.55), 2.518 (5.50), 2.523 (3.79), 2.540 (8.63), 2.664 (2.06), 2.669 (1.32), 2.673 (0.88), 2.994 (4.51), 5.107 (8.32), 5.960 (3.17), 7.134 (0.80), 7.144 (1.24), 7.146 (1.13), 7.152 (0.88), 7.195 (0.40), 7.263 (4.07), 7.266 (6.38), 7.268 (8.07), 7.274 (1.76), 7.279 (2.50), 7.282 (2.51), 7.306 (2.34), 7.577 (5.05), 7.582 (4.69).

### Intermediate 96

### 5-bromo-3-[(1-methyl-1H-pyrazol-5-yl)methoxy]pyridin-2-amine

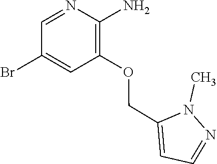 [SMILES: Cn1nccc1COc1cc(Br)cnc1N]

Intermediate 96 was prepared in analogy to Intermediate 95 using 5-bromo-3-fluoropyridin-2-amine (30.0 mg, 157 μmol) and (1-methyl-1H-pyrazol-5-yl)methanol (18.5 mg, 165 μmol).

LC-MS (method 1): Rₜ=0.92 min; MS (ESIpos): m/z=283 [M+H]⁺

¹H-NMR (400 MHz, DMSO-d₆): δ [ppm]=3.85 (s, 3H), 5.20 (s, 2H), 5.98 (s, 2H), 6.43 (d, 1H), 7.38 (dd, 2H), 7.60 (d, 1H).

### Intermediate 97

### 5-bromo-3-[(1S)-1-(1-methyl-1H-pyrazol-3-yl)ethoxy]-2-nitropyridine

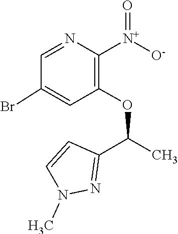 [SMILES: C[C@H](Oc1cc(Br)cnc1[N+](=O)[O-])c1ccn(C)n1]

Intermediate 97 was prepared in analogy to Intermediate 91 using 5-bromo-3-fluoro-2-nitropyridine (150 mg, 679 μmol) and (1S)-1-(1-methyl-1H-pyrazol-3-yl)ethan-1-ol (85.6 mg, 679 μmol).

LC-MS (method 1): Rt=1.10 min; MS (ESIpos): m/z=327 [M+H]⁺

¹H-NMR (400 MHz, CHLOROFORM-d): δ [ppm]=1.74 (d, 3H), 3.89 (s, 3H), 5.54 (d, 1H), 6.28 (d, 1H), 7.32 (d, 1H), 7.91 (d, 1H), 8.05 (d, 1H).

### Intermediate 98

### 5-bromo-3-[(1R)-1-(1-methyl-1H-pyrazol-3-yl)ethoxy]-2-nitropyridine

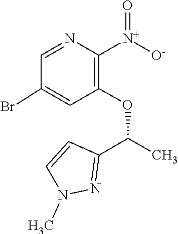 [SMILES: C[C@@H](Oc1cc(Br)cnc1[N+](=O)[O-])c1ccn(C)n1]

Intermediate 98 was prepared in analogy to Intermediate 91 using 5-bromo-3-fluoro-2-nitropyridine (150 mg, 679 μmol) and (1R)-1-(1-methyl-1H-pyrazol-3-yl)ethan-1-ol (85.6 mg, 679 μmol).

LC-MS (method 1): Rt=1.11 min; MS (ESIpos): m/z=327 [M+H]⁺

### Intermediate 99

### 5-bromo-3-[(1S)-1-phenylethoxy]pyridin-2-amine

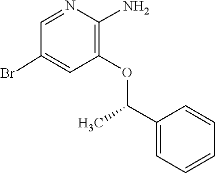 [SMILES: C[C@H](Oc1cc(Br)cnc1N)c1ccccc1]

Intermediate 99 was prepared in analogy to Intermediate 95 using 5-bromo-3-fluoropyridin-2-amine (298 mg, 1.56 mmol) and (1S)-1-phenylethan-1-ol (200 mg, 1.64 mmol).

LC-MS (method 1): Rt=1.22 min; MS (ESIpos): m/z=293 [M+H]⁺

1H NMR (400 MHz, DMSO-d6) δ ppm 1.55 (d, 3H) 5.57 (q, 1H) 6.03 (s, 2H) 7.05 (d, 1H) 7.22-7.29 (m, 1H) 7.31-7.39 (m, 2H) 7.42-7.47 (m, 2H) 7.49 (d, 1H)

### Intermediate 100

### 5-bromo-3-[(1R)-1-phenylethoxy]pyridin-2-amine

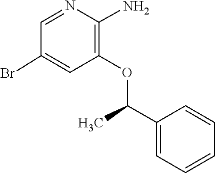 [SMILES: C[C@@H](Oc1cc(Br)cnc1N)c1ccccc1]

Intermediate 100 was prepared in analogy to Intermediate 95 using 5-bromo-3-fluoropyridin-2-amine (300 mg, 1.57 mmol) and (1R)-1-phenylethan-1-ol (200 μL, 1.6 mmol).

LC-MS (method 1): Rt=1.19 min; MS (ESIpos): m/z=293 [M+H]⁺

1H NMR (400 MHz, DMSO-d6) δ ppm 1.55 (d, 3H) 5.57 (q, 1H) 6.04 (s, 2H) 7.05 (d, 1H) 7.24-7.30 (m, 1H) 7.32-7.39 (m, 2H) 7.41-7.47 (m, 2H) 7.49 (d, 1H)

### Intermediate 101

### 3-(benzyloxy)-5-bromo-2-nitropyridine

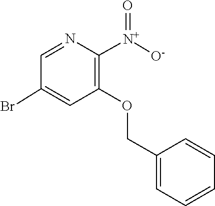 [SMILES: O=[N+]([O-])c1ncc(Br)cc1OCc1ccccc1]

5-bromo-2-nitropyridin-3-ol (2.00 g, 9.13 mmol) was solubilised in DMF (70 mL), Cs2CO3 (2.98 g, 9.13 mmol) was added and the mixture was stirred for 30 min at rt. (bromomethyl)benzene (1.1 mL, 9.1 mmol) was added and it was stirred for 2.5 h at 50° C. The mixture was diluted with water, stirred for 10 min, filtered and the precipitate was washed with water. It was dried under reduced pressure at 60° C. to give 2.70 g (98% purity, 94% yield) of the title compound, which was used without further purification.

¹H-NMR (400 MHz, DMSO-d6): δ [ppm]=5.40 (s, 2H), 7.35-7.41 (m, 1H), 7.43 (s, 2H), 7.44 (s, 2H), 8.31 (d, 1H), 8.43 (d, 1H).

LC-MS (method 1): Rₜ=1.29 min; MS (ESIpos): m/z=308 [M−H]⁺

### Intermediate 102

### (rac)-3-{[1-phenylethyl]sulfanyl}pyridin-2-amine

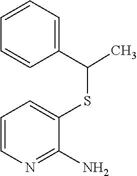 [SMILES: CC(Sc1cccnc1N)c1ccccc1]

(rac)-2-nitro-3-{[1-phenylethyl]sulfanyl}pyridine (716 mg, 2.75 mmol) and Fe (307 mg, 5.50 mmol) were stirred in methanol (5.4 mL)/acetic acid (5.4 mL) overnight at 85° C. The mixture was diluted with ethyl acetate, filtered and the precipitate was washed with ethyl acetate and dried under reduced pressure. The precipitate was solubilized in ethyl acetate, washed with sat. NaHCO3 solution and brine, dried over Na2SO4, filtered and concentrated under reduced pressure to give 627 mg (88% purity, 87% yield) of the title compound, which was used without further purification.

LC-MS (method 1): Rt=1.15 min; MS (ESIpos): m/z=232 [M+H]⁺

¹H-NMR (400 MHz, DMSO-d6): δ [ppm]=1.50 (d, 3H), 4.38 (q, 1H), 6.08 (s, 2H), 6.42 (dd, 1H), 7.17-7.23 (m, 1H), 7.24-7.32 (m, 5H), 7.87 (dd, 1H).

### Intermediate 103

### 5-bromo-3-[(cyclohexylmethyl)sulfanyl]pyridin-2-amine

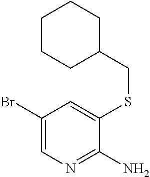 [SMILES: Nc1ncc(Br)cc1SCC1CCCCC1]

Intermediate 103 was prepared in analogy to Intermediate 102 using 5-bromo-3-[(cyclohexylmethyl)sulfanyl]-2-nitropyridine (1.00 g, 91% purity, 2.75 mmol).

¹H-NMR (400 MHz, DMSO-d6): δ [ppm]=0.91-1.04 (m, 2H), 1.08-1.24 (m, 3H), 1.34-1.46 (m, 1H), 1.55-1.63 (m, 1H), 1.63-1.71 (m, 2H), 1.77-1.84 (m, 2H), 2.76 (d, 2H), 6.20 (s, 2H), 7.61 (d, 1H), 7.90 (d, 1H).

LC-MS (method 1): Rt=1.52 min; MS (ESIpos): m/z=301 [M+H]⁺

### Intermediate 104

### 3-(benzylsulfanyl)-5-bromopyridin-2-amine

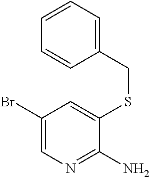 [SMILES: Nc1ncc(Br)cc1SCc1ccccc1]

Intermediate 104 was prepared in analogy to Intermediate 102 using 3-(benzylsulfanyl)-5-bromo-2-nitropyridine (224 mg, 689 μmol).

¹H-NMR (400 MHz, DMSO-d6): δ [ppm]=4.10 (s, 2H), 6.28 (s, 2H), 7.21-7.32 (m, 5H), 7.46 (d, 1H), 7.90 (d, 1H).

LC-MS (method 1): Rt=1.29 min; MS (ESIpos): m/z=296 [M+H]⁺

### Intermediate 105

### (rac)-5-bromo-3-{[1-cyclohexylethyl]sulfanyl}pyridin-2-amine

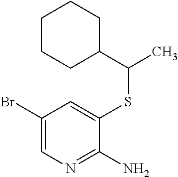 [SMILES: CC(Sc1cc(Br)cnc1N)C1CCCCC1]

Intermediate 105 was prepared in analogy to Intermediate 102 using (rac)-5-bromo-3-{[1-cyclohexylethyl]sulfanyl}-2-nitropyridine (455 mg, 1.32 mmol).

¹H-NMR (400 MHz, DMSO-d6): δ [ppm]=1.03-1.24 (m, 8H), 1.37-1.47 (m, 1H), 1.57-1.65 (m, 1H), 1.67-1.83 (m, 4H), 3.12-3.21 (m, 1H), 6.25 (s, 2H), 7.67 (d, 1H), 7.96 (d, 1H).

LC-MS (method 1): Rt=1.60 min; MS (ESIpos): m/z=315 [M+H]⁺

### Intermediate 106

### 5-bromo-3-[(1R)-1-(pyridin-3-yl)ethoxy]pyridin-2-amine

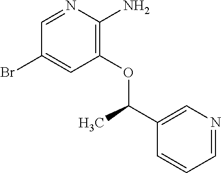 [SMILES: C[C@@H](Oc1cc(Br)cnc1N)c1cccnc1]

Intermediate 106 was prepared in analogy to Intermediate 102 using 5-bromo-2-nitro-3-[(1R)-1-(pyridin-3-yl)ethoxy]pyridine (760 mg, 32% purity, 750 μmol).

¹H-NMR (400 MHz, DMSO-d6): δ [ppm]=1.58 (d, 3H), 5.68 (q, 1H), 6.08 (s, 2H), 7.20 (d, 1H), 7.38 (dd, 1H), 7.52 (d, 1H), 7.90 (dt, 1H), 8.49 (dd, 1H), 8.69 (d, 1H).

LC-MS (method 1): Rt=0.89 min; MS (ESIpos): m/z=294 [M+H]⁺

### Intermediate 107

### 5-bromo-3-[(1S)-1-(pyridin-3-yl)ethoxy]pyridin-2-amine

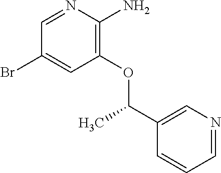 [SMILES: C[C@H](Oc1cc(Br)cnc1N)c1cccnc1]

Intermediate 107 was prepared in analogy to Intermediate 102 using 5-bromo-2-nitro-3-[(1S)-1-(pyridin-3-yl)ethoxy]pyridine (500 mg, 27% purity, 416 μmol).

¹H-NMR (400 MHz, DMSO-d6): δ [ppm]=1.58 (d, 3H), 5.68 (q, 1H), 6.08 (s, 2H), 7.20 (d, 1H), 7.38 (dd, 1H), 7.52 (d, 1H), 7.90 (dd, 1H), 8.49 (dd, 1H), 8.69 (d, 1H).

LC-MS (method 1): Rt=0.92 min; MS (ESIpos): m/z=294 [M+H]⁺

### Intermediate 108

### 5-bromo-3-[(cyclopentylmethyl)sulfanyl]pyridin-2-amine

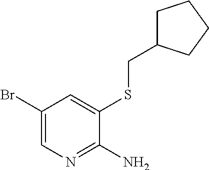 [SMILES: Nc1ncc(Br)cc1SCC1CCCC1]

Intermediate 108 was prepared in analogy to Intermediate 102 using 5-bromo-3-[(cyclopentylmethyl)sulfanyl]-2-nitropyridine (503 mg, 1.59 mmol).

¹H-NMR (400 MHz, DMSO-d6): δ [ppm]=1.19-1.30 (m, 2H), 1.42-1.65 (m, 4H), 1.71-1.81 (m, 2H), 1.96 (spt, 1H), 2.85 (d, 2H), 6.21 (s, 2H), 7.64 (d, 1H), 7.90 (d, 1H).

LC-MS (method 1): Rt=1.45 min; MS (ESIpos): m/z=287 [M+H]⁺

### Intermediate 109

### 3-(benzyloxy)-5-bromopyridin-2-amine

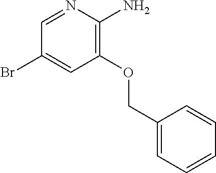 [SMILES: Nc1ncc(Br)cc1OCc1ccccc1]

Intermediate 109 was prepared in analogy to Intermediate 102 using 3-(benzyloxy)-5-bromo-2-nitropyridine (2.70 g, 8.73 mmol).

1H-NMR (400 MHz, DMSO-d6): δ [ppm]=5.16 (s, 2H), 5.97 (s, 2H), 7.28 (d, 1H), 7.30-7.42 (m, 5H), 7.58 (d, 1H).

LC-MS (method 1): Rₜ=1.19 min; MS (ESIneg): m/z=277 [M−H]⁻

### Intermediate 110

### 5-bromo-3-[(1R)-1-(pyridin-4-yl)ethoxy]pyridin-2-amine

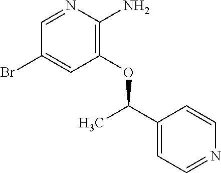 [SMILES: C[C@@H](Oc1cc(Br)cnc1N)c1ccncc1]

Intermediate 110 was prepared in analogy to Intermediate 102 using 5-bromo-2-nitro-3-[(1R)-1-(pyridin-4-yl)ethoxy]pyridine (1.60 g, 4.94 mmol).

LC-MS (method 2): Rt=0.57 min; MS (ESIpos): m/z=294 [M+H]⁺

¹H-NMR (400 MHz, DMSO-d₆): δ [ppm]=1.54 (d, 3H), 5.65 (q, 1H), 6.14 (br s, 2H), 7.11 (d, 1H), 7.48 (d, 2H), 7.53 (d, 1H), 8.56 (br d, 2H).

### Intermediate 111

### 5-bromo-3-[(1S)-1-(pyridin-4-yl)ethoxy]pyridin-2-amine

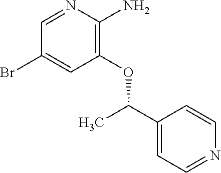 [SMILES: C[C@H](Oc1cc(Br)cnc1N)c1ccncc1]

Intermediate 111 was prepared in analogy to Intermediate 102 using 5-bromo-2-nitro-3-[(1S)-1-(pyridin-4-yl)ethoxy]pyridine (1.50 g, 4.63 mmol).

LC-MS (method 2): Rt=0.56 min; MS (ESIpos): m/z=294 [M+H]⁺

¹H-NMR (400 MHz, DMSO-d₆): δ [ppm]=1.54 (d, 3H), 5.64 (q, 1H), 6.12 (s, 2H), 7.10 (d, 1H), 7.42-7.48 (m, 2H), 7.53 (d, 1H), 8.51-8.58 (m, 2H).

### Intermediate 112

### 5-bromo-3-[(1R)-1-(2-chlorophenyl)ethoxy]pyridin-2-amine

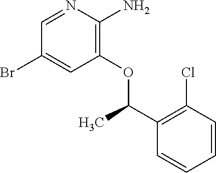 [SMILES: C[C@@H](Oc1cc(Br)cnc1N)c1ccccc1Cl]

Intermediate 112 was prepared in analogy to Intermediate 102 using 5-bromo-3-[(1R)-1-(2-chlorophenyl)ethoxy]-2-nitropyridine (1.00 g, 2.80 mmol).

LC-MS (method 1): Rₜ=1.31 min; MS (ESIpos): m/z=329 [M+H]⁺

H-NMR-Data ¹H NMR (DMSO-d₆) δ: 7.57 (m, 1H), 7.52 (d, 1H), 7.44-7.49 (m, 1H), 7.33 (quind, 2H), 6.80 (d, 1H), 6.13 (s, 2H), 5.73 (m, 1H), 1.58 (d, 3H)

### Intermediate 113

### 5-bromo-3-[(1S)-1-(2-chlorophenyl)ethoxy]pyridin-2-amine

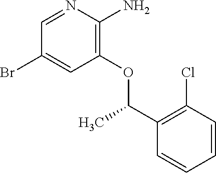 [SMILES: C[C@H](Oc1cc(Br)cnc1N)c1ccccc1Cl]

Intermediate 113 was prepared in analogy to Intermediate 102 using 5-bromo-3-[(1S)-1-(2-chlorophenyl)ethoxy]-2-nitropyridine (1.00 g, 2.80 mmol).

LC-MS (method 1): Rₜ=1.31 min; MS (ESIpos): m/z=329 [M+H]⁺

H-NMR-Data ¹H NMR (DMSO-d₆) δ: 7.57 (m, 1H), 7.52 (d, 1H), 7.46-7.49 (m, 1H), 7.34 (quind, 2H), 6.80 (d, 1H), 6.13 (s, 2H), 5.74 (m, 1H), 1.58 (d, 3H)

### Intermediate 114

### 5-bromo-3-[(1R)-1-(pyridin-2-yl)ethoxy]pyridin-2-amine

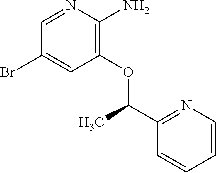 [SMILES: C[C@@H](Oc1cc(Br)cnc1N)c1ccccn1]

5-bromo-2-nitro-3-[(1R)-1-(pyridin-2-yl)ethoxy]pyridine (440 mg, 1.36 mmol) was solubilised in a degassed mixture of methanol (6.5 mL), water (13 mL) and THF (6.5 mL) under argon, Fe (379 mg, 6.79 mmol) and NH4Cl (363 mg, 6.79 mmol) were added and the mixture was stirred overnight at 80° C. (379 mg, 6.79 mmol) and (363 mg, 6.79 mmol) were added and the mixture was stirred for 5 h at 80° C. It was filtered and the filtrate was extracted 2× with ethyl acetate. The combined organic layers were washed with brine, dried over Na₂SO₄ and concentrated under reduced pressure. The residue was purified by flash chromatography to give 188 mg (47% yield) of the title compound.

1H NMR (DMSO-d6, 400 MHz) δ 8.5-8.6 (m, 1H), 7.81 (dt, 1H), 7.5-7.6 (m, 2H), 7.31 (ddd, 1H), 7.02 (d, 1H), 6.06 (s, 2H), 5.51 (q, 1H), 1.60 (d, 3H)

LC-MS (method 1): Rₜ=0.94 min; MS (ESIpos): m/z=296 [M+H]⁺

### Intermediate 115

### 3-[(2,6-dichlorophenyl)methoxy]pyridin-2-amine

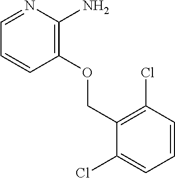 [SMILES: Nc1ncccc1OCc1c(Cl)cccc1Cl]

Intermediate 115 was prepared in analogy to Intermediate 114 using 3-[(2,6-dichlorophenyl)methoxy]-2-nitropyridine (600 mg, 2.01 mmol).

LC-MS (method 1): Rt=1.15 min; MS (ESIpos): m/z=269 [M+H]⁺

¹H-NMR (400 MHz, DMSO-d₆): δ [ppm]=5.21 (s, 2H), 5.50 (s, 2H), 6.53 (dd, 1H), 7.24 (dd, 1H), 7.43-7.51 (m, 1H), 7.53-7.59 (m, 3H).

### Intermediate 116

### 3-[(2-fluorophenyl)methoxy]pyridin-2-amine

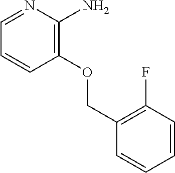 [SMILES: Nc1ncccc1OCc1ccccc1F]

Intermediate 116 was prepared in analogy to Intermediate 114 using 3-[(2-fluorophenyl)methoxy]-2-nitropyridine (830 mg, 3.34 mmol).

LC-MS (method 1): Rt=1.02 min; MS (ESIpos): m/z=219 [M+H]⁺

¹H-NMR (400 MHz, DMSO-d₆): δ [ppm]=5.15 (s, 2H), 5.71 (br s, 2H), 6.51 (dd, 1H), 7.15 (dd, 1H), 7.21-7.29 (m, 2H), 7.38-7.46 (m, 1H), 7.53 (dd, 1H), 7.64 (td, 1H).

### Intermediate 117

### 3-[(3-fluorophenyl)methoxy]pyridin-2-amine

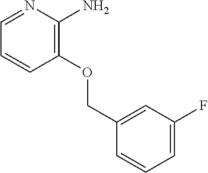 [SMILES: Nc1ncccc1OCc1cccc(F)c1]

Intermediate 117 was prepared in analogy to Intermediate 114 using 3-[(3-fluorophenyl)methoxy]-2-nitropyridine (757 mg, 3.05 mmol).

LC-MS (method 1): Rt=0.99 min; MS (ESIpos): m/z=219 [M+H]⁺

¹H-NMR (400 MHz, DMSO-d₆): δ [ppm]=5.14 (s, 2H), 5.83 (br s, 2H), 6.49 (dd, 1H), 7.09 (dd, 1H), 7.11-7.18 (m, 1H), 7.32 (d, 1H), 7.35-7.47 (m, 2H), 7.51 (dd, 1H).

### Intermediate 118

### 3-[(2-chlorophenyl)methoxy]pyridin-2-amine

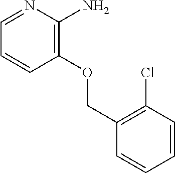 [SMILES: Nc1ncccc1OCc1ccccc1Cl]

Intermediate 118 was prepared in analogy to Intermediate 114 using 3-[(2-chlorophenyl)methoxy]-2-nitropyridine (600 mg, 2.27 mmol).

LC-MS (method 1): Rt=1.10 min; MS (ESIpos): m/z=235 [M+H]⁺

¹H-NMR (400 MHz, DMSO-d₆): δ [ppm]=5.16 (s, 2H), 5.68 (s, 2H), 6.46-6.53 (m, 1H), 7.11 (dd, 1H), 7.36-7.42 (m, 2H), 7.48-7.55 (m, 2H), 7.67-7.73 (m, 1H).

### Intermediate 119

### 3-[(pyridin-2-yl)methoxy]pyridin-2-amine

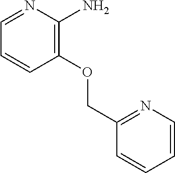 [SMILES: Nc1ncccc1OCc1ccccn1]

Intermediate 119 was prepared in analogy to Intermediate 114 using 2-nitro-3-[(pyridin-2-yl)methoxy]pyridine (865 mg, 3.74 mmol).

LC-MS (method 1): Rt=0.72 min; MS (ESIpos): m/z=202 [M+H]⁺

¹H-NMR (400 MHz, DMSO-d₆): δ [ppm]=5.17 (s, 2H), 5.77 (br s, 2H), 6.47 (dd, 1H), 7.07 (d, 1H), 7.34 (dd, 1H), 7.52 (d, 1H), 7.65 (d, 1H), 7.84 (td, 1H), 8.57 (d, 1H).

### Intermediate 120

### 3-[(2-chloro-5-fluorophenyl)methoxy]pyridin-2-amine

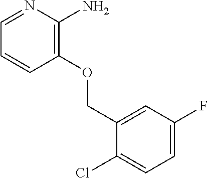 [SMILES: Nc1ncccc1OCc1cc(F)ccc1Cl]

Intermediate 120 was prepared in analogy to Intermediate 114 using 3-[(2-chloro-5-fluorophenyl)methoxy]-2-nitropyridine (1.01 g, 3.57 mmol).

LC-MS (method 1): Rt=1.09 min; MS (ESIpos): m/z=253 [M+H]⁺

¹H-NMR (400 MHz, DMSO-d₆): δ [ppm]=5.13 (s, 2H), 5.82 (s, 2H), 6.50 (dd, 1H), 7.12 (dd, 1H), 7.25 (td, 1H), 7.51-7.59 (m, 2H), 7.66 (dd, 1H).

### Intermediate 121

### 5-bromo-3-[(1S)-1-(pyridin-2-yl)ethoxy]pyridin-2-amine

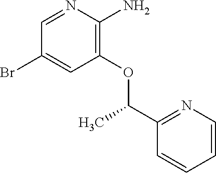 [SMILES: C[C@H](Oc1cc(Br)cnc1N)c1ccccn1]

Intermediate 121 was prepared in analogy to Intermediate 102 using 5-bromo-2-nitro-3-[(1S)-1-(pyridin-2-yl)ethoxy]pyridine (2.57 g, 20% purity, 1.59 mmol).

LC-MS (method 1): Rₜ=0.97 min; MS (ESIpos): m/z=296 [M+H]⁺

¹H-NMR (400 MHz, DMSO-d6) δ[ppm]: 1.590 (15.68), 1.606 (16.00), 1.895 (0.53), 1.986 (0.42), 2.518 (0.81), 2.523 (0.54), 3.159 (3.76), 3.173 (3.68), 4.099 (0.72), 4.112 (0.69), 5.482 (0.95), 5.497 (3.39), 5.514 (3.34), 5.530 (0.97), 6.066 (6.44), 7.014 (5.77), 7.019 (5.88), 7.297 (2.05), 7.299 (2.26), 7.309 (2.15), 7.311 (2.39), 7.315 (2.44), 7.318 (2.34), 7.327 (2.32), 7.330 (2.37), 7.501 (3.81), 7.521 (12.14), 7.526 (8.53), 7.785 (2.16), 7.790 (2.25), 7.804 (3.50), 7.809 (3.56), 7.824 (1.76), 7.829 (1.75), 8.544 (2.47), 8.546 (2.76), 8.548 (2.93), 8.551 (2.49), 8.556 (2.57), 8.558 (2.89), 8.560 (2.77), 8.563 (2.36).

### Intermediate 122

### (rac)-5-bromo-3-[1-(5-chloro-3-fluoropyridin-2-yl)ethoxy]pyridin-2-amine

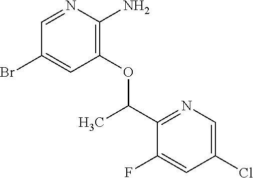 [SMILES: CC(Oc1cc(Br)cnc1N)c1ncc(Cl)cc1F]

(rac)-2-{1-[(5-bromo-2-nitropyridin-3-yl)oxy]ethyl}-5-chloro-3-fluoropyridine (215 mg, 571 μmol) and Fe (191 mg, 3.43 mmol) were stirred in acetic acid (1.5 mL)/ethanol (5.0 mL) for 4 h at 80° C. The mixture was evaporated, diluted with DCM/EtOH (9:1), washed with water and neutralized with K2CO3 solution (2M). The organic layer was dried and concentrated under reduced pressure. The residue was purified by flash chromatography to give 176 mg (90% purity, 80% yield) of the title compound.

LC-MS (method 1): Rt=1.19 min; MS (ESIpos): m/z=346 [M+H]⁺

¹H-NMR (400 MHz, DMSO-d₆): δ [ppm]=1.66 (d, 3H), 5.73-5.81 (m, 1H), 5.88 (s, 2H), 7.13 (d, 1H), 7.56 (d, 1H), 8.15 (dd, 1H), 8.56 (dd, 1H).

### Intermediate 123

### (rac)-5-bromo-3-[1-(pyrimidin-5-yl)ethoxy]pyridin-2-amine

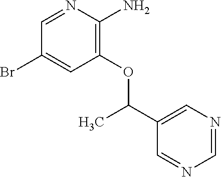 [SMILES: CC(Oc1cc(Br)cnc1N)c1cncnc1]

Intermediate 123 was prepared in analogy to Intermediate 122 using (rac)-5-{1-[(5-bromo-2-nitropyridin-3-yl)oxy]ethyl}pyrimidine (820 mg, 2.52 mmol).

LC-MS (method 1): Rt=0.80 min; MS (ESIpos): m/z=295 [M+H]⁺

¹H-NMR (400 MHz, DMSO-d₆): δ [ppm]=1.60 (d, 3H), 5.74 (q, 1H), 6.14 (s, 2H), 7.33 (d, 1H), 7.56 (d, 1H), 8.96 (s, 2H), 9.13 (s, 1H).

### Intermediate 124

### 5-bromo-3-{[1-(pyrimidin-5-yl)ethyl]oxy}pyridin-2-amine (Stereoisomer 1)

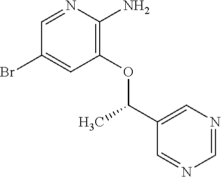 [SMILES: C[C@H](Oc1cc(Br)cnc1N)c1cncnc1]

The racemate (rac)-5-bromo-3-{[1-(pyrimidin-5-yl)ethyl]oxy}pyridin-2-amine (671 mg, 2.27 mmol) was separated by chiral HPLC to give 253.7 g (99% purity, 38% yield) of the target compound.

PrepCon Labomatic HPLC-4; Column: YMC Amylose SA 10μ, 250×50; eluent A: hexane+0.1 vol % diethylamine; eluent B: ethanol; isocratic: 60% A+40% B; flow: 100 mL/min; temperature: 25° C.; UV: 254 nm;

Retention time: 9.8-12.2 min

[α]20_{D}: −46.2° (c=1.00, DMSO) Instrument: Thermo Fisher UltiMate 3000; Column: YMC Amylose SA 3μ, 100×4.6; eluent A: hexane+0.1 vol % diethylamine; eluent B: ethanol; isocratic: 60% A+40% B; flow: 1.4 mL/min; temperature: 25° C.; UV: 254 nm;

¹H-NMR (400 MHz, DMSO-d₆): δ [ppm]=1.60 (d, 3H), 5.74 (q, 1H), 6.14 (s, 2H), 7.33 (d, 1H), 7.56 (d, 1H), 8.96 (s, 2H), 9.13 (s, 1H).

### Intermediate 125

### 5-bromo-3-{[1-(pyrimidin-5-yl)ethyl]oxy}pyridin-2-amine (Stereoisomer 2)

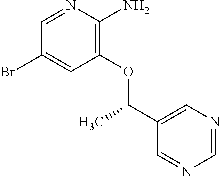 [SMILES: C[C@H](Oc1cc(Br)cnc1N)c1cncnc1]

The racemate (rac)-5-bromo-3-{[1-(pyrimidin-5-yl)ethyl]oxy}pyridin-2-amine (671 mg, 2.27 mmol) was separated by chiral HPLC to give 184.8 g (99% purity, 28% yield) of the target compound.

PrepCon Labomatic HPLC-4; Column: YMC Amylose SA 10μ, 250×50; eluent A: hexane+0.1 vol % diethylamine; eluent B: ethanol; isocratic: 60% A+40% B; flow: 100 mL/min; temperature: 25° C.; UV: 254 nm;

Retention time: 21.9-30.0 min;

[α]²⁰_{D}: +44.4° (c=1.00, DMSO) Instrument: Thermo Fisher UltiMate 3000; Column: YMC Amylose SA 3μ, 100×4.6; eluent A: hexane+0.1 vol % diethylamine; eluent B: ethanol; isocratic: 60% A+40% B; flow: 1.4 mL/min; temperature: 25° C.; UV: 254 nm;

¹H-NMR (400 MHz, DMSO-d₆): δ [ppm]=1.60 (d, 3H), 5.74 (q, 1H), 6.14 (s, 2H), 7.33 (d, 1H), 7.56 (d, 1H), 8.96 (s, 2H), 9.13 (s, 1H).

### Intermediate 126

### 5-bromo-3-[(1S)-1-(1-methyl-1H-pyrazol-3-yl)ethoxy]pyridin-2-amine

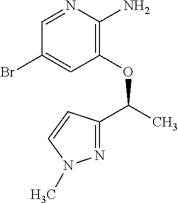 [SMILES: C[C@H](Oc1cc(Br)cnc1N)c1ccn(C)n1]

Intermediate 126 was prepared in analogy to Intermediate 122 using 5-bromo-3-[(1S)-1-(1-methyl-1H-pyrazol-3-yl)ethoxy]-2-nitropyridine (210 mg, 642 μmol).

LC-MS (method 1): Rₜ=0.92 min; MS (ESIpos): m/z=299 [M+H]⁺

### Intermediate 127

### 5-bromo-3-[(1R)-1-(1-methyl-1H-pyrazol-3-yl)ethoxy]pyridin-2-amine

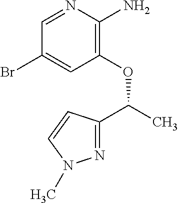 [SMILES: C[C@@H](Oc1cc(Br)cnc1N)c1ccn(C)n1]

Intermediate 127 was prepared in analogy to Intermediate 122 using 5-bromo-3-[(1R)-1-(1-methyl-1H-pyrazol-3-yl)ethoxy]-2-nitropyridine (211 mg, 645 μmol).

LC-MS (method 1): Rₜ=0.92 min; MS (ESIpos): m/z=299 [M+H]⁺

### Intermediate 128

### (rac)-5-bromo-3-{[1-phenylethyl]sulfanyl}pyridin-2-amine

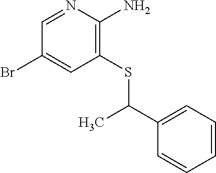 [SMILES: CC(Sc1cc(Br)cnc1N)c1ccccc1]

(rac)-3-{[1-phenylethyl]sulfanyl}pyridin-2-amine (600 mg, 2.60 mmol) was solubilised in acetonitrile (7.9 mL) under argon, cooled to −20° C., 1-bromopyrrolidine-2,5-dione (487 mg, 2.74 mmol), solubilised in acetonitrile (3.4 mL) was added and the mixture was stirred for 3 h h at rt. The mixture was diluted with DCM, washed with sat. Na2S2O3 solution and the aq. layer was extracted with DCM, dried over Na2SO4 and concentrated under reduced pressure. The residue was purified by preparative HPLC to give 336 mg (100% purity, 42% yield) of the title compound.

1H-NMR (400 MHz, DMSO-d6): δ [ppm]=1.52 (d, 3H), 4.48 (q, 1H), 6.35 (s, 2H), 7.20-7.25 (m, 1H), 7.26-7.33 (m, 4H), 7.38 (d, 1H), 7.92 (d, 1H).

LC-MS (method 1): Rt=1.34 min; MS (ESIpos): m/z=309 [M+H]⁺

### Intermediate 129

### 1-(2-amino-5-bromopyridin-3-yl)-2,2,2-trifluoroethan-1-one

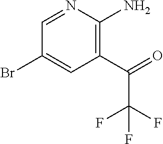 [SMILES: Nc1ncc(Br)cc1C(=O)C(F)(F)F]

Intermediate 129 was prepared in analogy to Intermediate 128 using 1-(2-aminopyridin-3-yl)-2,2,2-trifluoroethan-1-one-hydrogen chloride (1/1) (1.37 g, 6.05 mmol).

LC-MS (method 1): Rₜ=0.83 min; MS (ESIneg): m/z=269 [M−H]⁻

¹H-NMR (400 MHz, DMSO-d6) δ[ppm]: 2.518 (8.51), 2.522 (5.28), 3.313 (0.85), 6.326 (1.42), 6.471 (0.76), 7.656 (0.99), 7.662 (1.07), 7.964 (1.26), 7.969 (1.44), 7.988 (1.89), 7.993 (6.15), 7.998 (8.71), 8.003 (6.17), 8.014 (1.80), 8.019 (1.50), 8.043 (1.81), 8.048 (1.72), 8.157 (1.87), 8.233 (4.53), 8.515 (15.86), 8.521 (16.00).

### Intermediate 130

### 5-bromo-3-(cyclohexylmethoxy)pyridin-2-amine

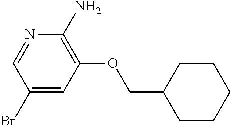 [SMILES: Nc1ncc(Br)cc1OCC1CCCCC1]

Intermediate 130 was prepared in analogy to Intermediate 128 using 3-(cyclohexylmethoxy)pyridin-2-amine (50.0 mg, 242 μmol).

LC-MS (method 1): Rt=1.42 min; MS (ESIpos): m/z=285 [M+H]⁺

1H-NMR (400 MHz, DMSO-d6) δ[ppm]: 1.008 (1.53), 1.026 (1.74), 1.054 (1.60), 1.084 (0.83), 1.155 (0.63), 1.209 (2.09), 1.234 (2.02), 1.266 (1.18), 1.658 (0.97), 1.691 (1.81), 1.725 (1.67), 1.804 (1.60), 1.835 (1.53), 1.988 (0.83), 2.332 (2.71), 2.518 (16.00), 2.522 (10.16), 2.673 (2.85), 3.305 (1.46), 3.405 (0.70), 3.770 (5.91), 3.786 (5.57), 4.221 (0.49), 5.869 (3.97), 6.239 (0.90), 7.136 (4.03), 7.141 (3.97), 7.542 (5.77), 7.547 (5.43).

### Intermediate 131

### 5-bromo-3-[(pyridin-3-yl)methoxy]pyridin-2-amine

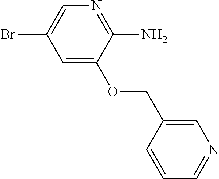 [SMILES: Nc1ncc(Br)cc1OCc1cccnc1]

Intermediate 131 was prepared in analogy to Intermediate 128 using 3-[(pyridin-3-yl)methoxy]pyridin-2-amine (500 mg, 2.48 mmol).

LC-MS (method 1): Rt=0.88 min; MS (ESIpos): m/z=280 [M+H]⁺

¹H-NMR (400 MHz, DMSO-d₆): δ [ppm]=5.20 (s, 2H), 6.03 (s, 2H), 7.34 (d, 1H), 7.43 (ddd, 1H), 7.60 (d, 1H), 7.94 (dt, 1H), 8.55 (dd, 1H), 8.70-8.73 (m, 1H).

### Intermediate 132

### 5-bromo-3-[(4-fluorophenyl)methoxy]pyridin-2-amine

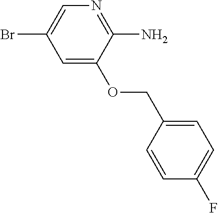 [SMILES: Nc1ncc(Br)cc1OCc1ccc(F)cc1]

Intermediate 132 was prepared in analogy to Intermediate 128 using 3-[(4-fluorophenyl)methoxy]pyridin-2-amine (160 mg, 733 μmol).

LC-MS (method 1): Rt=1.19 min; MS (ESIpos): m/z=297 [M+H]⁺

1H-NMR (400 MHz, DMSO-d6) δ[ppm]: 0.987 (0.77), 1.005 (1.78), 1.023 (0.81), 2.073 (0.65), 2.462 (0.47), 2.466 (0.50), 2.518 (0.96), 2.522 (0.61), 2.539 (16.00), 3.027 (0.52), 3.045 (0.51), 3.404 (0.84), 3.408 (0.78), 4.201 (0.51), 4.219 (0.72), 4.237 (0.49), 5.264 (8.18), 6.235 (1.20), 7.199 (4.05), 7.222 (0.47), 7.229 (3.01), 7.235 (1.10), 7.245 (1.28), 7.251 (5.68), 7.257 (1.21), 7.268 (1.06), 7.274 (3.32), 7.325 (4.89), 7.452 (3.99), 7.553 (0.42), 7.560 (2.78), 7.566 (1.20), 7.574 (3.06), 7.582 (2.73), 7.591 (1.04), 7.596 (2.34), 7.675 (3.57), 7.680 (3.75), 7.820 (4.86), 7.825 (4.44).

### Intermediate 133

### (rac)-5-bromo-3-[1-phenylethoxy]pyridin-2-amine

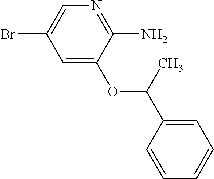 [SMILES: CC(Oc1cc(Br)cnc1N)c1ccccc1]

Intermediate 133 was prepared in analogy to Intermediate 128 using (rac)-3-(1-phenylethoxy)pyridin-2-amine (500 mg, 2.33 mmol).

LC-MS (method 1): Rt=1.23 min; MS (ESIpos): m/z=293 [M+H]⁺

¹H-NMR (400 MHz, DMSO-d₆): δ [ppm]=1.55 (d, 3H), 5.57 (q, 1H), 6.03 (s, 2H), 7.05 (d, 1H), 7.24-7.30 (m, 1H), 7.32-7.38 (m, 2H), 7.42-7.47 (m, 2H), 7.49 (d, 1H).

### Intermediate 134

### 5-bromo-3-[(trifluoromethyl)sulfanyl]pyridin-2-amine

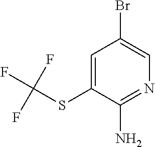 [SMILES: Nc1ncc(Br)cc1SC(F)(F)F]

Intermediate 134 was prepared in analogy to Intermediate 128 using 3-[(trifluoromethyl)sulfanyl]pyridin-2-amine (935 mg, 4.82 mmol).

LC-MS (method 1): Rt=1.19 min; MS (ESIpos): m/z=273 [M+H]⁺

¹H-NMR (400 MHz, DMSO-d₆): δ [ppm]=6.91 (s, 2H), 7.96 (d, 1H), 8.21 (d, 1H).

### Intermediate 135

### 5-bromo-3-(difluoromethoxy)pyridin-2-amine

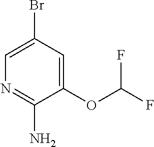 [SMILES: Nc1ncc(Br)cc1OC(F)F]

Intermediate 135 was prepared in analogy to Intermediate 128 using 3-(difluoromethoxy)pyridin-2-amine (3.00 g, 18.7 mmol).

LC-MS (method 1): Rt=0.95 min; MS (ESIpos): m/z=239 [M+H]⁺

¹H-NMR (400 MHz, DMSO-d₆): δ [ppm]=6.36 (s, 2H), 7.16 (br t, 1H), 7.51 (d, 1H), 7.89 (d, 1H).

### Intermediate 136

### 5-bromo-3-(trifluoromethoxy)pyridin-2-amine

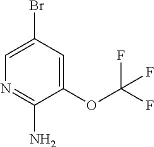 [SMILES: Nc1ncc(Br)cc1OC(F)(F)F]

Intermediate 136 was prepared in analogy to Intermediate 128 using 3-(trifluoromethoxy)pyridin-2-amine (500 mg, 2.81 mmol).

LC-MS (method 1): Rₜ=1.09 min; MS (ESIneg): m/z=255 [M−H]⁻

¹H-NMR (400 MHz, DMSO-d6) δ[ppm]: 2.518 (2.65), 2.523 (1.85), 2.669 (0.41), 6.694 (4.67), 7.748 (2.45), 7.752 (6.02), 7.755 (7.26), 7.757 (7.93), 7.760 (6.21), 8.031 (16.00), 8.036 (15.58).

### Intermediate 137

### 5-bromo-3-[(2,6-dichlorophenyl)methoxy]pyridin-2-amine

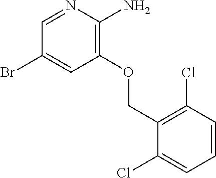 [SMILES: Nc1ncc(Br)cc1OCc1c(Cl)cccc1Cl]

Intermediate 137 was prepared in analogy to Intermediate 128 using 3-[(2,6-dichlorophenyl)methoxy]pyridin-2-amine (411 mg, 1.53 mmol).

LC-MS (method 1): Rt=1.30 min; MS (ESIpos): m/z=349 [M+H]⁺

¹H-NMR (400 MHz, DMSO-d₆): δ [ppm]=5.24 (s, 2H), 5.82 (s, 2H), 7.43-7.52 (m, 2H), 7.55-7.59 (m, 2H), 7.63 (d, 1H).

### Intermediate 138

### 5-bromo-3-[(2-fluorophenyl)methoxy]pyridin-2-amine

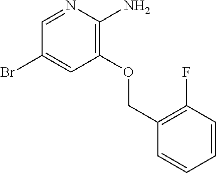 [SMILES: Nc1ncc(Br)cc1OCc1ccccc1F]

Intermediate 138 was prepared in analogy to Intermediate 128 using 3-[(2-fluorophenyl)methoxy]pyridin-2-amine (690 mg, 3.16 mmol).

LC-MS (method 1): Rt=1.19 min; MS (ESIpos): m/z=297 [M+H]⁺

¹H-NMR (400 MHz, DMSO-d₆): δ [ppm]=5.18 (s, 2H), 5.94 (s, 2H), 7.21-7.30 (m, 2H), 7.36 (d, 1H), 7.39-7.47 (m, 1H), 7.61 (d, 1H), 7.65 (td, 1H).

### Intermediate 139

### 5-bromo-3-[(3-fluorophenyl)methoxy]pyridin-2-amine

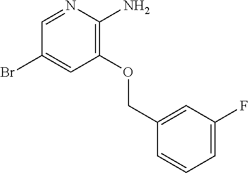 [SMILES: Nc1ncc(Br)cc1OCc1cccc(F)c1]

Intermediate 139 was prepared in analogy to Intermediate 128 using 3-[(3-fluorophenyl)methoxy]pyridin-2-amine (728 mg, 3.34 mmol).

LC-MS (method 1): Rt=1.18 min; MS (ESIpos): m/z=297 [M+H]⁺

¹H-NMR (400 MHz, DMSO-d₆): δ [ppm]=5.18 (s, 2H), 6.05 (s, 2H), 7.12-7.20 (m, 1H), 7.28 (d, 1H), 7.32 (d, 1H), 7.37-7.48 (m, 2H), 7.59 (d, 1H).

### Intermediate 140

### 5-bromo-3-[(2-chlorophenyl)methoxy]pyridin-2-amine

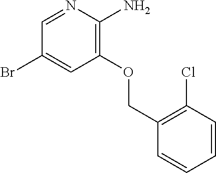 [SMILES: Nc1ncc(Br)cc1OCc1ccccc1Cl]

Intermediate 140 was prepared in analogy to Intermediate 128 using 3-[(2-chlorophenyl)methoxy]pyridin-2-amine (510 mg, 2.17 mmol).

LC-MS (method 1): Rt=1.27 min; MS (ESIpos): m/z=313 [M+H]⁺

¹H-NMR (400 MHz, DMSO-d₆): δ [ppm]=5.19 (s, 2H), 5.97 (s, 2H), 7.34 (d, 1H), 7.37-7.44 (m, 2H), 7.48-7.55 (m, 1H), 7.62 (d, 1H), 7.68-7.75 (m, 1H).

### Intermediate 141

### 5-bromo-3-[(pyridin-2-yl)methoxy]pyridin-2-amine

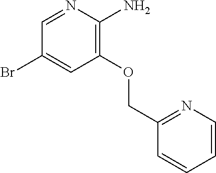 [SMILES: Nc1ncc(Br)cc1OCc1ccccn1]

Intermediate 141 was prepared in analogy to Intermediate 128 using 3-[(pyridin-2-yl)methoxy]pyridin-2-amine (594 mg, 2.95 mmol).

LC-MS (method 1): Rt=0.92 min; MS (ESIpos): m/z=280 [M+H]⁺

¹H-NMR (400 MHz, DMSO-d₆): δ [ppm]=5.21 (s, 2H), 6.06 (s, 2H), 7.29 (d, 1H), 7.35 (ddd, 1H), 7.60 (d, 1H), 7.67 (d, 1H), 7.85 (td, 1H), 8.55-8.60 (m, 1H).

### Intermediate 142

### 5-bromo-3-[(2-chloro-5-fluorophenyl)methoxy]pyridin-2-amine

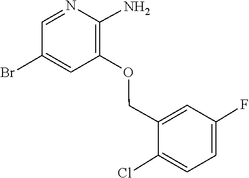 [SMILES: Nc1ncc(Br)cc1OCc1cc(F)ccc1Cl]

Intermediate 142 was prepared in analogy to Intermediate 128 using 3-[(2-chloro-5-fluorophenyl)methoxy]pyridin-2-amine (900 mg, 3.56 mmol).

LC-MS (method 1): Rt=1.23 min; MS (ESIpos): m/z=331 [M+H]⁺

¹H-NMR (400 MHz, DMSO-d₆): δ [ppm]=5.17 (s, 2H), 6.10 (s, 2H), 7.27 (td, 1H), 7.36 (d, 1H), 7.56 (dd, 1H), 7.63 (d, 1H), 7.70 (dd, 1H).

### Intermediate 143

### 5-bromo-3-(difluoromethyl)pyridin-2-amine

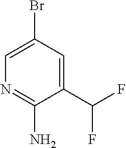 [SMILES: Nc1ncc(Br)cc1C(F)F]

Intermediate 143 was prepared in analogy to Intermediate 128 using 3-(difluoromethyl)pyridin-2-amine (500 mg, 3.47 mmol).

### Intermediate 144

### 5-bromo-3-phenylpyridin-2-amine

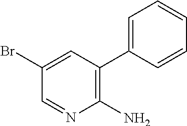 [SMILES: Nc1ncc(Br)cc1-c1ccccc1]

5-bromo-3-iodopyridin-2-amine (250 mg, 836 μmol) was solubilised in acetonitrile (2.5 mL), phenylboronic acid (120 mg, 987 μmol), Pd(PPh3)2Cl2 (29.4 mg, 41.8 μmol) and Na2CO3 (2.5 mL, 2.0 M, 5.0 mmol) were added and the mixture was stirred for 1 h at 60° C. The mixture was diluted with ethyl acetate/THF (1:1), washed with brine and the organic layer was dried over a silicone filter and evaporated. The residue was purified by flash chromatography to give 175 mg (84% yield) of the title compound.

LC-MS (method 2): Rt=1.02 min; MS (ESIpos): m/z=249 [M+H]⁺

1H-NMR (400 MHz, DMSO-d6): δ [ppm]=5.84 (s, 2H), 7.35-7.43 (m, 1H), 7.43-7.52 (m, 5H), 8.02 (d, 1H).

### Intermediate 145

### 5-bromo-3-(4-methylphenyl)pyridin-2-amine

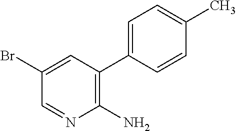

Intermediate 145 was prepared in analogy to Intermediate 144 using 5-bromo-3-iodopyridin-2-amine (250 mg, 836 μmol) and (4-methylphenyl)boronic acid (134 mg, 987 μmol).

LC-MS (method 1): Rt=1.26 min; MS (ESIpos): m/z=265 [M+H]⁺

1H-NMR (400 MHz, DMSO-d6): δ [ppm]=2.35 (s, 3H), 5.80 (s, 2H), 7.24-7.30 (m, 2H), 7.31-7.37 (m, 2H), 7.43 (d, 1H), 8.00 (d, 1H).

### Intermediate 146

### 5-bromo-3-(2-methylphenyl)pyridin-2-amine

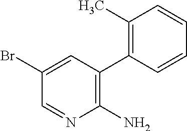

Intermediate 146 was prepared in analogy to Intermediate 144 using 5-bromo-3-iodopyridin-2-amine (250 mg, 836 μmol) and (2-methylphenyl)boronic acid (134 mg, 987 μmol).

LC-MS (method 1): Rt=1.22 min; MS (ESIpos): m/z=263 [M+H]⁺

¹H-NMR (400 MHz, DMSO-d₆): δ [ppm]=2.11 (s, 3H), 5.55 (s, 2H), 7.14 (d, 1H), 7.23-7.30 (m, 1H), 7.31-7.33 (m, 2H), 7.36 (d, 1H), 8.03 (d, 1H).

### Intermediate 147

### 5-bromo-3-(3-methylphenyl)pyridin-2-amine

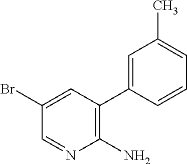

Intermediate 147 was prepared in analogy to Intermediate 144 using 5-bromo-3-iodopyridin-2-amine (250 mg, 836 μmol) and (3-methylphenyl)boronic acid (134 mg, 987 μmol).

LC-MS (method 1): Rt=1.26 min; MS (ESIpos): m/z=265 [M+H]⁺

¹H-NMR (400 MHz, DMSO-d₆): δ [ppm]=2.35 (s, 3H), 5.83 (s, 2H), 7.17-7.28 (m, 3H), 7.32-7.38 (m, 1H), 7.44 (d, 1H), 8.01 (d, 1H).

### Intermediate 148

### 5-bromo-3-(3-fluorophenyl)pyridin-2-amine

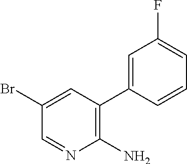 [SMILES: Nc1ncc(Br)cc1-c1cccc(F)c1]

Intermediate 148 was prepared in analogy to Intermediate 144 using 5-bromo-3-iodopyridin-2-amine (250 mg, 836 μmol) and (3-fluorophenyl)boronic acid (138 mg, 987 μmol).

LC-MS (method 2): Rt=1.11 min; MS (ESIpos): m/z=267 [M+H]⁺

¹H-NMR (400 MHz, DMSO-d₆): δ [ppm]=5.97 (s, 2H), 7.18-7.25 (m, 1H), 7.27-7.32 (m, 2H), 7.46-7.54 (m, 2H), 7.54-7.66 (m, 1H), 8.03 (d, 1H).

### Intermediate 149

### 5-bromo[3,3′-bipyridin]-2-amine

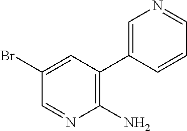 [SMILES: Nc1ncc(Br)cc1-c1cccnc1]

5-bromo-3-iodopyridin-2-amine (250 mg, 836 μmol) was solubilised in acetonitrile (2.5 mL), pyridin-3-ylboronic acid (121 mg, 987 μmol), Pd(PPh3)2Cl2 (29.4 mg, 41.8 μmol) and Na2CO3 (2.5 mL, 2.0 M, 5.0 mmol) were added and the mixture was stirred for 1 h at 60° C. pyridin-3-ylboronic acid (121 mg, 987 μmol), Pd(PPh3)2Cl2 (29.4 mg, 41.8 μmol) and Cs2CO3 (273 mg, 836 μmol) were added and the mixture was stirred for 1 h at 60° C. The mixture was diluted with ethyl acetate/THF (1:1), washed with brine and the organic layer was dried over a silicone filter and evaporated. The residue was purified by flash chromatography to give 225 mg of the title compound.

LC-MS (method 1): Rt=0.87 min; MS (ESIpos): m/z=252 [M+H]⁺

¹H-NMR (400 MHz, DMSO-d₆): δ [ppm]=6.02 (s, 2H), 7.47 (ddd, 1H), 7.54 (d, 1H), 7.87 (dt, 1H), 8.05 (d, 1H), 8.58 (dd, 1H), 8.62 (dd, 1H).

### Intermediate 150

### 5-bromo-3-(2-fluorophenyl)pyridin-2-amine

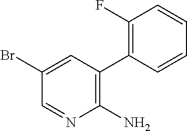 [SMILES: Nc1ncc(Br)cc1-c1ccccc1F]

Intermediate 150 was prepared in analogy to Intermediate 144 using 5-bromo-3-iodopyridin-2-amine (250 mg, 836 μmol) and (2-fluorophenyl)boronic acid (138 mg, 987 μmol).

LC-MS (method 1): Rt=1.16 min; MS (ESIpos): m/z=267 [M+H]⁺

¹H-NMR (400 MHz, DMSO-d₆): δ [ppm]=5.86 (s, 2H), 7.25-7.34 (m, 2H), 7.39 (td, 1H), 7.43-7.51 (m, 2H), 8.06 (d, 1H).

### Intermediate 151

### 5-bromo[3,4′-bipyridin]-2-amine

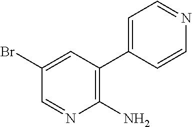 [SMILES: Nc1ncc(Br)cc1-c1ccncc1]

Intermediate 151 was prepared in analogy to Intermediate 149 using 5-bromo-3-iodopyridin-2-amine (250 mg, 836 μmol) and pyridin-4-ylboronic acid (121 mg, 987 μmol).

LC-MS (method 1): Rt=0.88 min; MS (ESIpos): m/z=250 [M+H]⁺

¹H-NMR (400 MHz, DMSO-d₆): δ [ppm]=6.11 (s, 2H), 7.46-7.51 (m, 2H), 7.58 (d, 1H), 8.08 (d, 1H), 8.60-8.66 (m, 2H).

### Intermediate 152

### 5-bromo-3-[3-(methanesulfonyl)phenyl]pyridin-2-amine

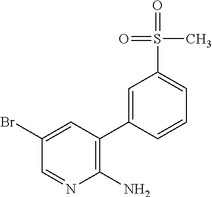 [SMILES: CS(=O)(=O)c1cccc(-c2cc(Br)cnc2N)c1]

Intermediate 152 was prepared in analogy to Intermediate 144 using 5-bromo-3-iodopyridin-2-amine (250 mg, 836 μmol) and [3-(methanesulfonyl)phenyl]boronic acid (197 mg, 987 μmol).

LC-MS (method 1): Rₜ=0.96 min; MS (ESIpos): m/z=327 [M+H]⁺

¹H-NMR (400 MHz, DMSO-d₆): δ [ppm]=3.27 (s, 3H), 6.08 (s, 2H), 7.60 (d, 1H), 7.70-7.76 (m, 1H), 7.78-7.84 (m, 1H), 7.92 (dt, 1H), 7.94-7.96 (m, 1H), 8.07 (d, 1H).

### Intermediate 153

### 5-bromo-3-[4-(methanesulfonyl)phenyl]pyridin-2-amine

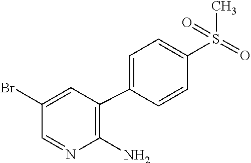 [SMILES: CS(=O)(=O)c1ccc(-c2cc(Br)cnc2N)cc1]

Intermediate 153 was prepared in analogy to Intermediate 144 using 5-bromo-3-iodopyridin-2-amine (250 mg, 836 μmol) and [4-(methanesulfonyl)phenyl]boronic acid (197 mg, 987 μmol).

LC-MS (method 1): Rt=0.96 min; MS (ESIpos): m/z=327 [M+H]⁺

¹H-NMR (400 MHz, DMSO-d₆): δ [ppm]=3.25 (s, 3H), 6.06 (s, 2H), 7.56 (d, 1H), 7.71-7.73 (m, 1H), 7.73-7.75 (m, 1H), 7.97-7.99 (m, 1H), 7.99-8.01 (m, 1H), 8.07 (d, 1H).

### Intermediate 154

### 1-[4-(2-amino-5-bromopyridin-3-yl)phenyl]ethan-1-one

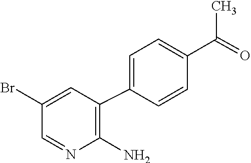 [SMILES: CC(=O)c1ccc(-c2cc(Br)cnc2N)cc1]

Intermediate 154 was prepared in analogy to Intermediate 144 using 5-bromo-3-iodopyridin-2-amine (250 mg, 836 μmol) and (4-acetylphenyl)boronic acid (162 mg, 987 μmol).

LC-MS (method 1): Rt=1.09 min; MS (ESIpos): m/z=293 [M+H]⁺

### Intermediate 155

### 5-bromo-3-[4-(methanesulfinyl)phenyl]pyridin-2-amine

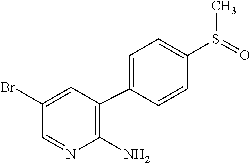 [SMILES: CS(=O)c1ccc(-c2cc(Br)cnc2N)cc1]

Intermediate 155 was prepared in analogy to Intermediate 144 using 5-bromo-3-iodopyridin-2-amine (250 mg, 836 μmol) and [4-(methanesulfinyl)phenyl]boronic acid (182 mg, 987 μmol).

LC-MS (method 1): Rt=0.88 min; MS (ESIpos): m/z=311 [M+H]⁺

### Intermediate 156

### 5-bromo-3-(3-methoxyphenyl)pyridin-2-amine

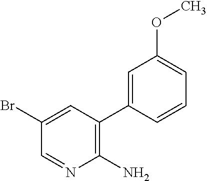 [SMILES: COc1cccc(-c2cc(Br)cnc2N)c1]

Intermediate 156 was prepared in analogy to Intermediate 144 using 5-bromo-3-iodopyridin-2-amine (250 mg, 836 μmol) and (3-methoxyphenyl)boronic acid (150 mg, 987 μmol).

LC-MS (method 1): Rt=1.18 min; MS (ESIpos): m/z=281 [M+H]⁺

¹H-NMR (400 MHz, DMSO-d₆): δ [ppm]=3.80 (s, 3H), 5.86 (s, 2H), 6.92-7.03 (m, 3H), 7.38 (t, 1H), 7.48 (d, 1H), 8.01 (d, 1H).

### Intermediate 157

### 5-bromo-3-[4-(cyclopropanesulfonyl)phenyl]pyridin-2-amine

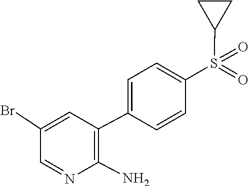 [SMILES: Nc1ncc(Br)cc1-c1ccc(S(=O)(=O)C2CC2)cc1]

Intermediate 157 was prepared in analogy to Intermediate 144 using 5-bromo-3-iodopyridin-2-amine (250 mg, 836 μmol) and [4-(cyclopropanesulfonyl)phenyl]boronic acid (223 mg, 987 μmol).

LC-MS (method 1): Rt=1.07 min; MS (ESIpos): m/z=355 [M+H]⁺

¹H-NMR (400 MHz, DMSO-d₆): δ [ppm]=1.03-1.11 (m, 2H), 1.12-1.20 (m, 2H), 2.89 (tt, 1H), 6.08 (s, 2H), 7.58 (d, 1H), 7.71-7.76 (m, 2H), 7.93-7.97 (m, 2H), 8.07 (d, 1H).

### Intermediate 158

### 4-(2-amino-5-bromopyridin-3-yl)benzene-1-sulfonamide

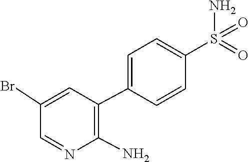 [SMILES: Nc1ncc(Br)cc1-c1ccc(S(N)(=O)=O)cc1]

Intermediate 158 was prepared in analogy to Intermediate 144 using 5-bromo-3-iodopyridin-2-amine (250 mg, 836 μmol) and (4-sulfamoylphenyl)boronic acid (198 mg, 987 μmol).

LC-MS (method 1): Rt=0.87 min; MS (ESIpos): m/z=280 [M+H]⁺

¹H-NMR (400 MHz, DMSO-d₆): δ [ppm]=6.02 (s, 2H), 7.44 (s, 2H), 7.53 (d, 1H), 7.63-7.68 (m, 2H), 7.86-7.90 (m, 2H), 8.06 (d, 1H).

### Intermediate 159

### 4-(2-amino-5-bromopyridin-3-yl)-N-methylbenzene-1-sulfonamide

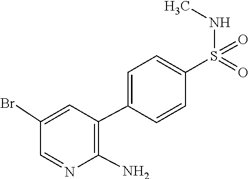 [SMILES: CNS(=O)(=O)c1ccc(-c2cc(Br)cnc2N)cc1]

Intermediate 159 was prepared in analogy to Intermediate 144 using 5-bromo-3-iodopyridin-2-amine (250 mg, 836 μmol) and [4-(methylsulfamoyl)phenyl]boronic acid (212 mg, 987 μmol).

LC-MS (method 1): Rt=0.98 min; MS (ESIpos): m/z=342 [M+H]⁺

### Intermediate 160

### 4-(2-amino-5-bromopyridin-3-yl)-N, N-dimethylbenzene-1-sulfonamide

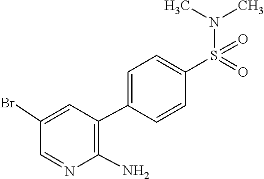 [SMILES: CN(C)S(=O)(=O)c1ccc(-c2cc(Br)cnc2N)cc1]

Intermediate 160 was prepared in analogy to Intermediate 144 using 5-bromo-3-iodopyridin-2-amine (250 mg, 836 μmol) and [4-(dimethylsulfamoyl)phenyl]boronic acid (226 mg, 987 μmol).

LC-MS (method 1): Rt=1.11 min; MS (ESIpos): m/z=358 [M+H]⁺

¹H-NMR (400 MHz, DMSO-d₆): δ [ppm]=2.62-2.69 (m, 6H), 6.08 (s, 2H), 7.56 (d, 1H), 7.68-7.73 (m, 2H), 7.77-7.82 (m, 2H), 8.07 (d, 1H).

### Intermediate 161

### 5-bromo-3-(1-methyl-1H-pyrazol-3-yl)pyridin-2-amine

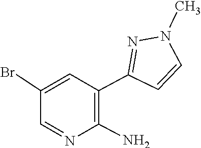

Intermediate 161 was prepared in analogy to Intermediate 144 using 5-bromo-3-iodopyridin-2-amine (250 mg, 836 μmol) and 1-methyl-3-(4,4,5,5-tetramethyl-1,3,2-dioxaborolan-2-yl)-1H-pyrazole (205 mg, 987 μmol).

LC-MS (method 1): Rt=1.00 min; MS (ESIpos): m/z=255 [M+H]⁺

¹H-NMR (400 MHz, DMSO-d₆): δ [ppm]=3.91 (s, 3H), 6.91 (d, 1H), 7.22 (br s, 2H), 7.80 (d, 1H), 7.96 (d, 1H), 8.05 (d, 1H).

### Intermediate 162

### 5-bromo-3-(1,2-thiazol-5-yl)pyridin-2-amine

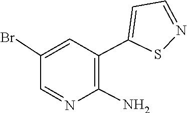 [SMILES: Nc1ncc(Br)cc1-c1ccns1]

Intermediate 162 was prepared in analogy to Intermediate 144 using 5-bromo-3-iodopyridin-2-amine (250 mg, 836 μmol) and 5-(4,4,5,5-tetramethyl-1,3,2-dioxaborolan-2-yl)-1,2-thiazole (208 mg, 987 μmol).

LC-MS (method 1): Rt=0.98 min; MS (ESIpos): m/z=258 [M+H]⁺

¹H-NMR (400 MHz, DMSO-d₆): δ [ppm]=6.28 (s, 2H), 7.72 (d, 1H), 7.83 (d, 1H), 8.13 (d, 1H), 8.63 (d, 1H).

### Intermediate 163

### 5-bromo-3-(1,5-dimethyl-1H-pyrazol-4-yl)pyridin-2-amine

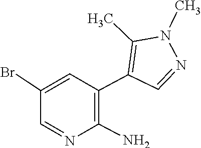

Intermediate 163 was prepared in analogy to Intermediate 144 using 5-bromo-3-iodopyridin-2-amine (250 mg, 836 μmol) and (1,5-dimethyl-1H-pyrazol-4-yl)boronic acid (138 mg, 987 μmol).

LC-MS (method 1): Rt=0.90 min; MS (ESIpos): m/z=269 [M+H]⁺

¹H-NMR (400 MHz, DMSO-d₆): δ [ppm]=2.15 (s, 3H), 3.76 (s, 3H), 5.76 (s, 2H), 7.34 (d, 1H), 7.41 (s, 1H), 7.95 (d, 1H).

### Intermediate 164

### 5-bromo-1′-methyl-1′,2′,3′,6′-tetrahydro[3,4′-bipyridin]-2-amine

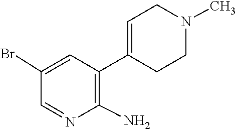

Intermediate 164 was prepared in analogy to Intermediate 144 using 5-bromo-3-iodopyridin-2-amine (250 mg, 836 μmol) and 1-methyl-4-(4,4,5,5-tetramethyl-1,3,2-dioxaborolan-2-yl)-1,2,3,6-tetrahydropyridine (224 mg, 1.00 mmol).

1H-NMR (400 MHz, CHLOROFORM-d): δ [ppm]=2.34-2.48 (m, 5H), 2.65 (t, 2H), 3.08 (q, 2H), 4.59 (br s, 2H), 5.86 (tt, 1H), 7.33 (d, 1H), 8.00 (d, 1H).

LC-MS (method 1): Rₜ=0.97 min; MS (ESIpos): m/z=270 [M+H]⁺

### Intermediate 165

### 5-(4,4,5,5-tetramethyl-1,3,2-dioxaborolan-2-yl)-3-(trifluoromethyl)pyridin-2-amine

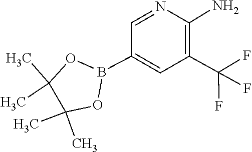 [SMILES: CC1(C)OB(c2cnc(N)c(C(F)(F)F)c2)OC1(C)C]

5-Bromo-3-(trifluoromethyl)pyridin-2-amine (2.00 g, 8.30 mmol) and 4,4,4′,4′,5,5,5′,5′-octamethyl-2,2′-bi-1,3,2-dioxaborolane (2.53 g, 9.96 mmol) were solubilised in dioxane (43 mL), potassium acetate (2.44 g, 24.9 mmol) and Pd(dppf)Cl₂ (607 mg, 830 μmol) were added and the mixture was stirred overnight at 100° C. The mixture was diluted with dichloromethane, filtered and evaporated. The residue was purified by flash chromatography to give 2.70 g of the target compound.

1H-NMR (400 MHz, DMSO-d6) δ[ppm]: 1.052 (0.88), 1.065 (16.00), 1.069 (1.16), 1.145 (0.90), 1.154 (8.40), 1.163 (1.61), 1.176 (0.70), 1.269 (11.09), 1.290 (1.05), 3.332 (0.74), 3.936 (2.59), 7.799 (0.47), 7.803 (0.48), 7.939 (0.72), 8.375 (0.46), 8.377 (0.46).

### Intermediate 166

### 2-amino-5-(4,4,5,5-tetramethyl-1,3,2-dioxaborolan-2-yl)pyridine-3-carbonitrile

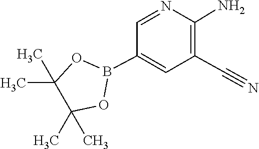 [SMILES: CC1(C)OB(c2cnc(N)c(C#N)c2)OC1(C)C]

2-Amino-5-bromopyridine-3-carbonitrile (1.40 g, 7.07 mmol), 4,4,4′,4′,5,5,5′,5′-octamethyl-2,2′-bi-1,3,2-dioxaborolane (1.97 g, 7.78 mmol), Pd(dppf)Cl₂ (289 mg, 353 μmol) and potassium acetate (2.08 g, 21.2 mmol) were stirred in degassed dioxane (35 mL) at 100° C. overnight. The reaction mixture was filtered and used without further work up or purification in the next step.

¹H NMR (400 MHz, DMSO-d₆) δ ppm 1.27 (s, 12H) 7.33 (s, 2H) 7.92 (d, 1H) 8.37 (d, 1H).

### Intermediate 167

### 3-fluoro-5-(4,4,5,5-tetramethyl-1,3,2-dioxaborolan-2-yl)pyridin-2-amine

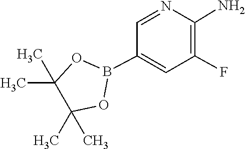 [SMILES: CC1(C)OB(c2cnc(N)c(F)c2)OC1(C)C]

5-bromo-3-fluoropyridin-2-amine (75.2 mg, 394 μmol), 4,4,4′,4′,5,5,5′,5′-octamethyl-2,2′-bi-1,3,2-dioxaborolane (110 mg, 433 μmol), Pd(dppf)Cl₂ (16.1 mg, 19.7 μmol), dppf (10.9 mg, 19.7 μmol) and KOAc (116 mg, 1.18 mmol) were stirred in degassed 1,4-dioxane (2.0 mL) overnight at 100° C. The mixture was filtered and the filtrate was concentrated under reduced pressure to give 124 mg of the title compound, which was used without further purification.

LC-MS (method 1): Rt=0.54 min; MS (ESIpos): m/z=239 [M+H]⁺

¹H-NMR (400 MHz, DMSO-d6) δ[ppm]: 1.066 (16.00), 1.260 (3.09).

### Intermediate 168

### 1-[2-amino-5-(4,4,5,5-tetramethyl-1,3,2-dioxaborolan-2-yl)pyridin-3-yl]-2,2,2-trifluoroethan-1-one

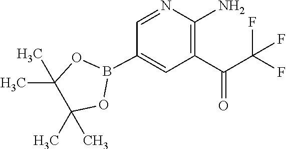 [SMILES: CC1(C)OB(c2cnc(N)c(C(=O)C(F)(F)F)c2)OC1(C)C]

Intermediate 168 was prepared in analogy to Intermediate 167 using 1-(2-amino-5-bromopyridin-3-yl)-2,2,2-trifluoroethan-1-one (37.0 mg, 138 μmol).

LC-MS (method 1): Rt=0.72 min; MS (ESIpos): m/z=317 [M+H]⁺

### Intermediate 169

### (rac)-(6-amino-5-{[1-phenylethyl]sulfanyl}pyridin-3-yl)boronic acid

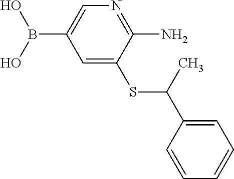 [SMILES: CC(Sc1cc(B(O)O)cnc1N)c1ccccc1]

Intermediate 169 was prepared in analogy to Intermediate 167 using (rac)-5-bromo-3-{[1-phenylethyl]sulfanyl}pyridin-2-amine (50.0 mg, 162 μmol).

LC-MS (method 1): Rt=0.79 min; MS (ESIpos): m/z=275 [M+H]⁺

### Intermediate 170

### 3-[(1R)-1-(2,6-dichloro-3-fluorophenyl)ethoxy]-5-(4,4,5,5-tetramethyl-1,3,2-dioxaborolan-2-yl)pyridin-2-amine

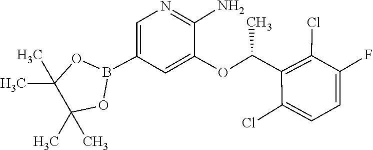 [SMILES: C[C@@H](Oc1cc(B2OC(C)(C)C(C)(C)O2)cnc1N)c1c(Cl)ccc(F)c1Cl]

5-bromo-3-[(1R)-1-(2,6-dichloro-3-fluorophenyl)ethoxy]pyridin-2-amine (500 mg, 1.32 mmol), 4,4,4′,4′,5,5,5′,5′-octamethyl-2,2′-bi-1,3,2-dioxaborolane (368 mg, 1.45 mmol), Pd(dppf)Cl2 (53.7 mg, 65.8 μmol) and KOAc (387 mg, 3.9 mmol) were stirred in degassed 1,4-dioxane (13 mL) for 5 h at 100° C. The mixture was filtered and the filtrate was concentrated under reduced pressure to give 560 mg (100% yield) of the title compound, which was used without further purification.

LC-MS (method 1): Rₜ=1.38 min; MS (ESIpos): m/z=427 [M+H]⁺

### Intermediate 171

### [6-amino-5-(2-hydroxypropan-2-yl)pyridin-3-yl]boronic acid

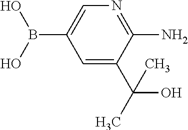

Intermediate 171 was prepared in analogy to Intermediate 170 using 2-(2-amino-5-bromopyridin-3-yl)propan-2-ol (300 mg, 1.30 mmol).

LC-MS (method 1): Rt=0.74 min; MS (ESIpos): m/z=197 [M+H]⁺

¹H-NMR (400 MHz, DMSO-d6) δ[ppm]: 1.066 (8.83), 1.156 (16.00), 1.164 (1.43), 1.255 (6.11), 1.276 (1.08), 1.292 (1.21), 1.466 (2.93), 1.492 (0.50), 3.566 (6.26), 3.941 (1.54), 5.409 (0.67), 6.469 (0.43), 6.548 (0.50), 7.943 (1.30).

### Intermediate 172

### {6-amino-5-[(cyclopentylmethyl)sulfanyl]pyridin-3-yl}boronic acid

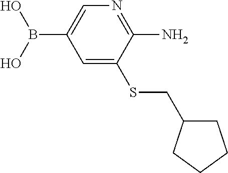 [SMILES: Nc1ncc(B(O)O)cc1SCC1CCCC1]

Intermediate 172 was prepared in analogy to Intermediate 170 using 5-bromo-3-[(cyclopentylmethyl)sulfanyl]pyridin-2-amine (79.0 mg, 275 μmol).

LC-MS (method 1): Rt=0.82 min; MS (ESIpos): m/z=253 [M+H]⁺

### Intermediate 173

### 3-(benzyloxy)-5-(4,4,5,5-tetramethyl-1,3,2-dioxaborolan-2-yl)pyridin-2-amine

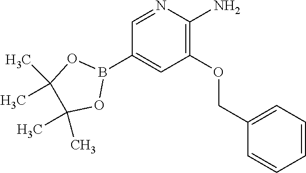 [SMILES: CC1(C)OB(c2cnc(N)c(OCc3ccccc3)c2)OC1(C)C]

Intermediate 173 was prepared in analogy to Intermediate 170 using 3-(benzyloxy)-5-bromopyridin-2-amine (1.92 g, 6.88 mmol).

### Intermediate 174

### methyl 2-amino-5-(4,4,5,5-tetramethyl-1,3,2-dioxaborolan-2-yl)pyridine-3-carboxylate

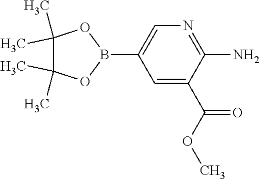 [SMILES: COC(=O)c1cc(B2OC(C)(C)C(C)(C)O2)cnc1N]

Intermediate 174 was prepared in analogy to Intermediate 170 using methyl 2-amino-5-bromopyridine-3-carboxylate (2.00 g, 8.66 mmol).

1H-NMR (400 MHz, DMSO-d6) δ[ppm]: 1.066 (14.18), 1.156 (16.00), 1.164 (1.99), 1.274 (7.97), 1.292 (0.30), 1.898 (0.69), 2.332 (0.23), 2.518 (1.32), 2.523 (0.79), 2.673 (0.23), 3.566 (0.16), 3.810 (0.62), 3.822 (3.20), 3.938 (1.56), 7.524 (0.55), 7.941 (0.63), 8.274 (0.48), 8.279 (0.55), 8.377 (0.58), 8.381 (0.56).

### Intermediate 175

### [6-amino-5-(methylsulfanyl)pyridin-3-yl]boronic acid

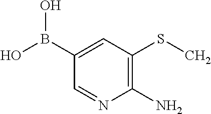

Intermediate 175 was prepared in analogy to Intermediate 170 using 5-bromo-3-(methylsulfanyl)pyridin-2-amine (680 mg, 3.10 mmol).

1H-NMR (400 MHz, DMSO-d6) δ[ppm]: 1.035 (0.65), 1.052 (1.36), 1.065 (12.03), 1.070 (1.06), 1.156 (16.00), 1.164 (15.62), 1.179 (0.19), 1.261 (3.77), 1.274 (0.23), 1.292 (0.84), 1.310 (0.30), 1.907 (0.31), 2.344 (1.86), 2.354 (0.33), 2.518 (0.44), 2.523 (0.29), 3.422 (0.27), 3.435 (0.28), 3.439 (0.27), 3.452 (0.28), 3.938 (1.80), 4.343 (0.19), 4.356 (0.37), 4.368 (0.18), 6.423 (0.21), 7.578 (0.24), 7.582 (0.24), 7.940 (1.14), 8.086 (0.23), 8.090 (0.22).

### Intermediate 176

### [6-amino-5-(3-fluorophenyl)pyridin-3-yl]boronic acid

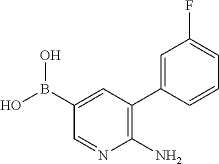 [SMILES: Nc1ncc(B(O)O)cc1-c1cccc(F)c1]

Intermediate 176 was prepared in analogy to Intermediate 170 using 5-bromo-3-(3-fluorophenyl)pyridin-2-amine (119 mg, 446 μmol).

LC-MS (method 2): Rt=0.57 min; MS (ESIpos): m/z=233 [M+H]⁺

### Intermediate 177

### [6-amino-5-(2-fluorophenyl)pyridin-3-yl]boronic acid

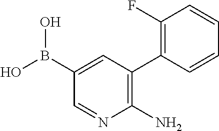 [SMILES: Nc1ncc(B(O)O)cc1-c1ccccc1F]

Intermediate 177 was prepared in analogy to Intermediate 170 using 5-bromo-3-(2-fluorophenyl)pyridin-2-amine (227 mg, 850 μmol).

LC-MS (method 2): Rt=0.55 min; MS (ESIpos): m/z=233 [M+H]⁺

### Intermediate 178

### [6-amino-5-(2-methoxyphenyl)pyridin-3-yl]boronic acid

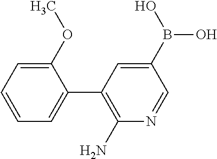 [SMILES: COc1ccccc1-c1cc(B(O)O)cnc1N]

Intermediate 178 was prepared in analogy to Intermediate 170 using 5-bromo-3-(2-methoxyphenyl)pyridin-2-amine (85.0 mg, 305 μmol).

LC-MS (method 2): Rt=0.58 min; MS (ESIpos): m/z=245 [M+H]⁺

### Intermediate 179

### {6-amino-5-[3-(methanesulfonyl)phenyl]pyridin-3-yl}boronic acid

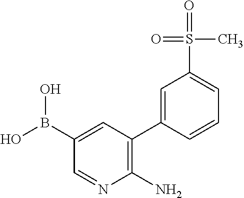 [SMILES: CS(=O)(=O)c1cccc(-c2cc(B(O)O)cnc2N)c1]

Intermediate 179 was prepared in analogy to Intermediate 170 using 5-bromo-3-[3-(methanesulfonyl)phenyl]pyridin-2-amine (100 mg, 306 μmol).

LC-MS (method 2): Rt=0.46 min; MS (ESIpos): m/z=293 [M+H]⁺

### Intermediate 180

### [6-amino-5-(phenylsulfanyl)pyridin-3-yl]boronic acid

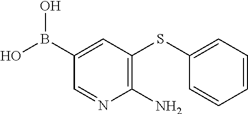 [SMILES: Nc1ncc(B(O)O)cc1Sc1ccccc1]

Intermediate 180 was prepared in analogy to Intermediate 170 using 5-bromo-3-(phenylsulfanyl)pyridin-2-amine (1.40 g, 4.98 mmol).

LC-MS (method 1): Rt=0.59 min; MS (ESIpos): m/z=247 [M+H]⁺

### Intermediate 181

### {6-amino-5-[4-(methanesulfonyl)phenyl]pyridin-3-yl}boronic acid

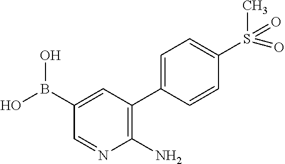 [SMILES: CS(=O)(=O)c1ccc(-c2cc(B(O)O)cnc2N)cc1]

Intermediate 181 was prepared in analogy to Intermediate 170 using 5-bromo-3-[4-(methanesulfonyl)phenyl]pyridin-2-amine (200 mg, 611 μmol).

LC-MS (method 2): Rt=0.44 min; MS (ESIpos): m/z=293 [M+H]⁺

### Intermediate 182

### [5-(4-acetylphenyl)-6-aminopyridin-3-yl]boronic acid

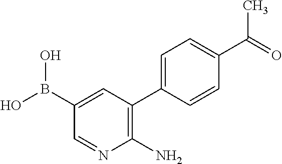 [SMILES: CC(=O)c1ccc(-c2cc(B(O)O)cnc2N)cc1]

Intermediate 182 was prepared in analogy to Intermediate 170 using 1-[4-(2-amino-5-bromopyridin-3-yl)phenyl]ethan-1-one (240 mg, 824 μmol).

LC-MS (method 2): Rt=0.52 min; MS (ESIpos): m/z=257 [M+H]⁺

### Intermediate 183

### {6-amino-5-[4-(methanesulfinyl)phenyl]pyridin-3-yl}boronic acid

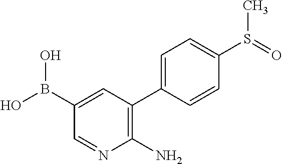 [SMILES: CS(=O)c1ccc(-c2cc(B(O)O)cnc2N)cc1]

Intermediate 183 was prepared in analogy to Intermediate 170 using 5-bromo-3-[4-(methanesulfinyl)phenyl]pyridin-2-amine (240 mg, 771 μmol).

LC-MS (method 2): Rt=0.43 min; MS (ESIpos): m/z=277 [M+H]⁺

### Intermediate 184

### [6-amino-5-(3-methoxyphenyl)pyridin-3-yl]boronic acid

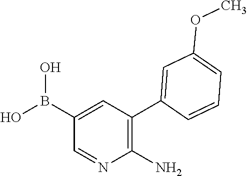 [SMILES: COc1cccc(-c2cc(B(O)O)cnc2N)c1]

Intermediate 184 was prepared in analogy to Intermediate 170 using 5-bromo-3-(3-methoxyphenyl)pyridin-2-amine (200 mg, 716 μmol).

LC-MS (method 2): Rt=0.61 min; MS (ESIpos): m/z=245 [M+H]⁺

### Intermediate 185

### {6-amino-5-[4-(cyclopropanesulfonyl)phenyl]pyridin-3-yl}boronic acid

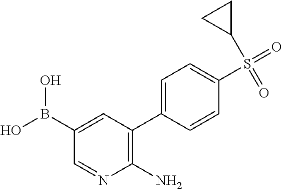 [SMILES: Nc1ncc(B(O)O)cc1-c1ccc(S(=O)(=O)C2CC2)cc1]

Intermediate 185 was prepared in analogy to Intermediate 170 using 5-bromo-3-[4-(cyclopropanesulfonyl)phenyl]pyridin-2-amine (220 mg, 623 μmol).

LC-MS (method 2): Rt=0.55 min; MS (ESIpos): m/z=319 [M+H]⁺

### Intermediate 186

### [6-amino-5-(4-sulfamoylphenyl)pyridin-3-yl]boronic acid

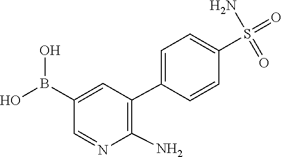 [SMILES: Nc1ncc(B(O)O)cc1-c1ccc(S(N)(=O)=O)cc1]

Intermediate 186 was prepared in analogy to Intermediate 170 using 4-(2-amino-5-bromopyridin-3-yl)benzene-1-sulfonamide (150 mg, 457 μmol).

LC-MS (method 2): Rt=0.46 min; MS (ESIpos): m/z=294 [M+H]⁺

### Intermediate 187

### {6-amino-5-[4-(methylsulfamoyl)phenyl]pyridin-3-yl}boronic acid

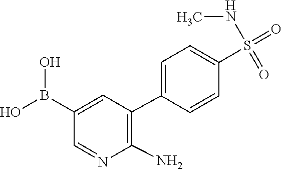

Intermediate 187 was prepared in analogy to Intermediate 170 using 4-(2-amino-5-bromopyridin-3-yl)-N,N-dimethylbenzene-1-sulfonamide (220 mg, 618 μmol).

LC-MS (method 2): Rt=0.47 min; MS (ESIpos): m/z=308 [M+H]⁺

### Intermediate 188

### {6-amino-5-[4-(dimethylsulfamoyl)phenyl]pyridin-3-yl}boronic acid

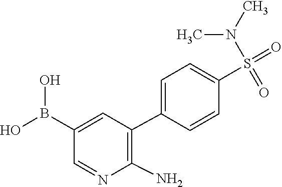 [SMILES: CN(C)S(=O)(=O)c1ccc(-c2cc(B(O)O)cnc2N)cc1]

Intermediate 188 was prepared in analogy to Intermediate 170 using 4-(2-amino-5-bromopyridin-3-yl)-N,N-dimethylbenzene-1-sulfonamide (150 mg, 421 μmol).

LC-MS (method 2): Rt=0.57 min; MS (ESIpos): m/z=322 [M+H]⁺

### Intermediate 189

### [6-amino-5-(cyclohexylmethoxy)pyridin-3-yl]boronic acid

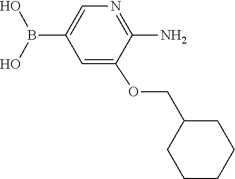 [SMILES: Nc1ncc(B(O)O)cc1OCC1CCCCC1]

Intermediate 189 was prepared in analogy to Intermediate 170 using 5-bromo-3-(cyclohexylmethoxy)pyridin-2-amine (45.0 mg, 158 μmol).

LC-MS (method 2): Rt=0.80 min; MS (ESIpos): m/z=251 [M+H]⁺

### Intermediate 190

### {6-amino-5-[(pyridin-3-yl)methoxy]pyridin-3-yl}boronic acid

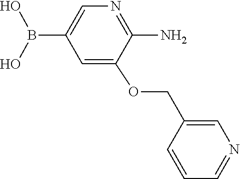 [SMILES: Nc1ncc(B(O)O)cc1OCc1cccnc1]

Intermediate 190 was prepared in analogy to Intermediate 170 using 5-bromo-3-[(pyridin-3-yl)methoxy]pyridin-2-amine (100 mg, 357 μmol).

LC-MS (method 1): Rt=0.63 min; MS (ESIpos): m/z=246 [M+H]⁺

### Intermediate 191

### {6-amino-5-[(4-fluorophenyl)methoxy]pyridin-3-yl}boronic acid

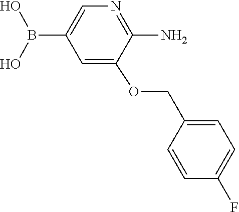 [SMILES: Nc1ncc(B(O)O)cc1OCc1ccc(F)cc1]

Intermediate 191 was prepared in analogy to Intermediate 170 using 5-bromo-3-[(4-fluorophenyl)methoxy]pyridin-2-amine (30.0 mg, 101 μmol).

LC-MS (method 1): Rt=0.65 min; MS (ESIpos): m/z=263 [M+H]⁺

### Intermediate 192

### (rac)-[6-amino-5-(1-phenylethoxy)pyridin-3-yl]boronic acid

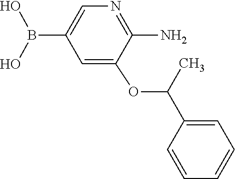 [SMILES: CC(Oc1cc(B(O)O)cnc1N)c1ccccc1]

Intermediate 192 was prepared in analogy to Intermediate 170 using (rac)-5-bromo-3-(1-phenylethoxy)pyridin-2-amine (95.0 mg, 324 μmol).

LC-MS (method 1): Rt=0.74 min; MS (ESIpos): m/z=259 [M+H]⁺

### Intermediate 193

### [6-amino-5-(piperidin-1-yl)pyridin-3-yl]boronic acid

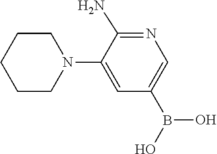 [SMILES: Nc1ncc(B(O)O)cc1N1CCCCC1]

Intermediate 193 was prepared in analogy to Intermediate 170 using 5-bromo-3-(piperidin-1-yl)pyridin-2-amine (125 mg, 488 μmol).

LC-MS (method 1): Rt=0.64 min; MS (ESIpos): m/z=222 [M+H]⁺

### Intermediate 194

### {6-amino-5-[(1R)-1-(pyridin-4-yl)ethoxy]pyridin-3-yl}boronic acid

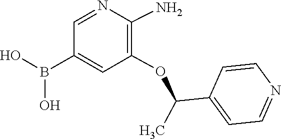 [SMILES: C[C@@H](Oc1cc(B(O)O)cnc1N)c1ccncc1]

Intermediate 194 was prepared in analogy to Intermediate 170 using 5-bromo-3-[(1R)-1-(pyridin-4-yl)ethoxy]pyridin-2-amine (60.0 mg, 204 μmol).

LC-MS (method 1): Rt=0.47 min; MS (ESIpos): m/z=260 [M+H]⁺

### Intermediate 195

### {6-amino-5-[(1S)-1-(pyridin-4-yl)ethoxy]pyridin-3-yl}boronic acid

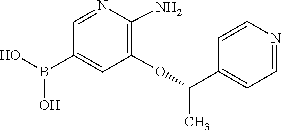 [SMILES: C[C@H](Oc1cc(B(O)O)cnc1N)c1ccncc1]

Intermediate 195 was prepared in analogy to Intermediate 170 using 5-bromo-3-[(1S)-1-(pyridin-4-yl)ethoxy]pyridin-2-amine (100 mg, 340 μmol).

LC-MS (method 1): Rt=0.44 min; MS (ESIpos): m/z=260 [M+H]⁺

### Intermediate 196

### [6-amino-5-(1-methyl-1H-pyrazol-3-yl)pyridin-3-yl]boronic acid

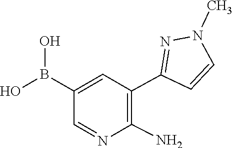 [SMILES: Cn1ccc(-c2cc(B(O)O)cnc2N)n1]

Intermediate 196 was prepared in analogy to Intermediate 170 using 5-bromo-3-(1-methyl-1H-pyrazol-3-yl)pyridin-2-amine (150 mg, 593 μmol).

LC-MS (method 2): Rt=0.49 min; MS (ESIpos): m/z=219 [M+H]⁺

### Intermediate 197

### [6-amino-5-(1,2-thiazol-5-yl)pyridin-3-yl]boronic acid

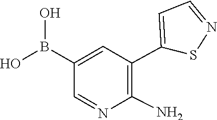 [SMILES: Nc1ncc(B(O)O)cc1-c1ccns1]

Intermediate 197 was prepared in analogy to Intermediate 170 using 5-bromo-3-(1,2-thiazol-5-yl)pyridin-2-amine (120 mg, 469 μmol).

LC-MS (method 2): Rt=0.44 min; MS (ESIpos): m/z=222 [M+H]⁺

### Intermediate 198

### [6-amino-5-(1,5-dimethyl-1H-pyrazol-4-yl)pyridin-3-yl]boronic acid

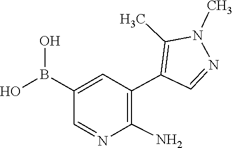 [SMILES: Cc1c(-c2cc(B(O)O)cnc2N)cnn1C]

Intermediate 198 was prepared in analogy to Intermediate 170 using 5-bromo-3-(1,5-dimethyl-1H-pyrazol-4-yl)pyridin-2-amine (100 mg, 374 μmol).

LC-MS (method 2): Rt=0.45 min; MS (ESIpos): m/z=233 [M+H]⁺

### Intermediate 199

### {6-amino-5-[(trifluoromethyl)sulfanyl]pyridin-3-yl}boronic acid

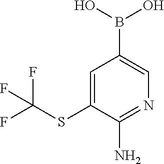 [SMILES: Nc1ncc(B(O)O)cc1SC(F)(F)F]

Intermediate 199 was prepared in analogy to Intermediate 170 using 5-bromo-3-[(trifluoromethyl)sulfanyl]pyridin-2-amine (1.25 g, 4.58 mmol).

LC-MS (method 2): Rt=0.57 min; MS (ESIpos): m/z=239 [M+H]⁺

### Intermediate 200

### 3-[(1R)-1-(2-chlorophenyl)ethoxy]-5-(4,4,5,5-tetramethyl-1,3,2-dioxaborolan-2-yl)pyridin-2-amine

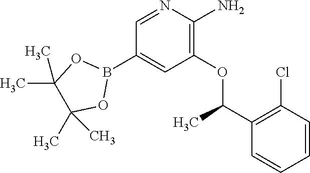 [SMILES: C[C@@H](Oc1cc(B2OC(C)(C)C(C)(C)O2)cnc1N)c1ccccc1Cl]

Intermediate 200 was prepared in analogy to Intermediate 170 using 5-bromo-3-[(1R)-1-(2-chlorophenyl)ethoxy]pyridin-2-amine (154 mg, 470 μmol).

LC-MS (method 1): Rt=1.32 min; MS (ESIpos): m/z=375 [M+H]⁺

### Intermediate 201

### 3-[(1S)-1-(2-chlorophenyl)ethoxy]-5-(4,4,5,5-tetramethyl-1,3,2-dioxaborolan-2-yl)pyridin-2-amine

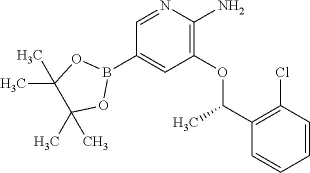 [SMILES: C[C@H](Oc1cc(B2OC(C)(C)C(C)(C)O2)cnc1N)c1ccccc1Cl]

Intermediate 201 was prepared in analogy to Intermediate 170 using 5-bromo-3-[(1S)-1-(2-chlorophenyl)ethoxy]pyridin-2-amine (87.0 mg, 266 μmol).

LC-MS (method 1): Rt=1.33 min; MS (ESIpos): m/z=375 [M+H]⁺

### Intermediate 202

### 3-[(1R)-1-(pyridin-2-yl)ethoxy]-5-(4,4,5,5-tetramethyl-1,3,2-dioxaborolan-2-yl)pyridin-2-amine

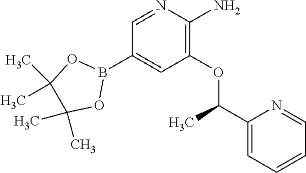 [SMILES: C[C@@H](Oc1cc(B2OC(C)(C)C(C)(C)O2)cnc1N)c1ccccn1]

Intermediate 202 was prepared in analogy to Intermediate 170 using 5-bromo-3-[(1R)-1-(pyridin-2-yl)ethoxy]pyridin-2-amine (250 mg, 90% purity, 765 μmol).

LC-MS (method 1): Rt=0.87 min; MS (ESIpos): m/z=342 [M+H]⁺

### Intermediate 203

### 3-{[1-(pyrimidin-5-yl)ethyl]oxy}-5-(4,4,5,5-tetramethyl-1,3,2-dioxaborolan-2-yl)pyridin-2-amine (Stereoisomer 1)

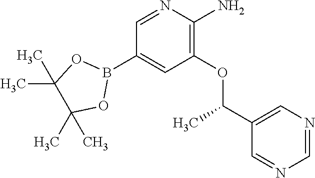 [SMILES: C[C@H](Oc1cc(B2OC(C)(C)C(C)(C)O2)cnc1N)c1cncnc1]

Intermediate 203 was prepared in analogy to Intermediate 170 using 5-bromo-3-{[1-(pyrimidin-5-yl)ethyl]oxy}pyridin-2-amine (100 mg, 339 μmol, stereoisomer 1).

LC-MS (method 1): Rt=0.63 min; MS (ESIpos): m/z=343 [M+H]⁺

### Intermediate 204

### 3-{[1-(pyrimidin-5-yl)ethyl]oxy}-5-(4,4,5,5-tetramethyl-1,3,2-dioxaborolan-2-yl)pyridin-2-amine (Stereoisomer 2)

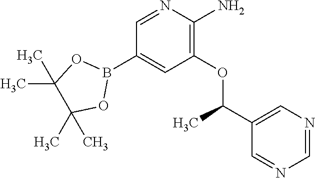 [SMILES: C[C@@H](Oc1cc(B2OC(C)(C)C(C)(C)O2)cnc1N)c1cncnc1]

Intermediate 204 was prepared in analogy to Intermediate 170 using 5-bromo-3-{[1-(pyrimidin-5-yl)ethyl]oxy}pyridin-2-amine (100 mg, 339 μmol, stereoisomer 2).

LC-MS (method 1): Rt=0.63 min; MS (ESIpos): m/z=343 [M+H]⁺

### Intermediate 205

### [6-amino-5-(difluoromethoxy)pyridin-3-yl]boronic acid

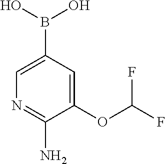 [SMILES: Nc1ncc(B(O)O)cc1OC(F)F]

Intermediate 205 was prepared in analogy to Intermediate 170 using 5-bromo-3-(difluoromethoxy)pyridin-2-amine (3.00 g, 12.6 mmol).

LC-MS (method 2): Rt=0.46 min; MS (ESIpos): m/z=205 [M+H]⁺

### Intermediate 206

### [6-amino-5-(trifluoromethoxy)pyridin-3-yl]boronic acid

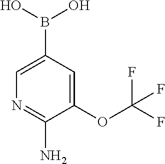 [SMILES: Nc1ncc(B(O)O)cc1OC(F)(F)F]

Intermediate 206 was prepared in analogy to Intermediate 170 using 5-bromo-3-(trifluoromethoxy)pyridin-2-amine (3.60 g, 14.0 mmol).

LC-MS (method 2): Rt=0.50 min; MS (ESIpos): m/z=223 [M+H]⁺

### Intermediate 207

### 3-[(2,6-dichlorophenyl)methoxy]-5-(4,4,5,5-tetramethyl-1,3,2-dioxaborolan-2-yl)pyridin-2-amine

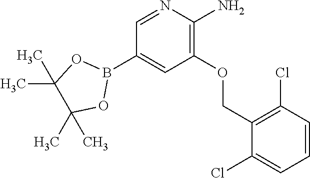 [SMILES: CC1(C)OB(c2cnc(N)c(OCc3c(Cl)cccc3Cl)c2)OC1(C)C]

Intermediate 207 was prepared in analogy to Intermediate 170 using 5-bromo-3-[(2,6-dichlorophenyl)methoxy]pyridin-2-amine (393 mg, 1.13 mmol).

LC-MS (method 1): Rt=1.32 min; MS (ESIpos): m/z=395 [M+H]⁺

### Intermediate 208

### 3-[(2-fluorophenyl)methoxy]-5-(4,4,5,5-tetramethyl-1,3,2-dioxaborolan-2-yl)pyridin-2-amine

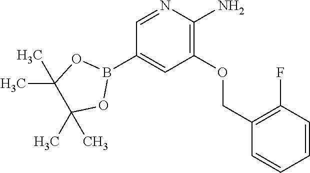 [SMILES: CC1(C)OB(c2cnc(N)c(OCc3ccccc3F)c2)OC1(C)C]

Intermediate 208 was prepared in analogy to Intermediate 170 using 5-bromo-3-[(2-fluorophenyl)methoxy]pyridin-2-amine (373 mg, 1.26 mmol).

LC-MS (method 1): Rt=1.18 min; MS (ESIpos): m/z=345 [M+H]⁺

### Intermediate 209

### 3-[(3-fluorophenyl)methoxy]-5-(4,4,5,5-tetramethyl-1,3,2-dioxaborolan-2-yl)pyridin-2-amine

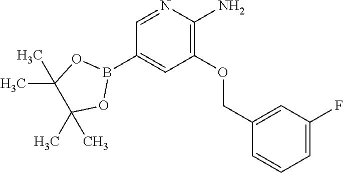 [SMILES: CC1(C)OB(c2cnc(N)c(OCc3cccc(F)c3)c2)OC1(C)C]

Intermediate 209 was prepared in analogy to Intermediate 170 using 5-bromo-3-[(3-fluorophenyl)methoxy]pyridin-2-amine (345 mg, 1.16 mmol).

LC-MS (method 1): Rt=1.15 min; MS (ESIpos): m/z=345 [M+H]⁺

### Intermediate 210

### 3-[(2-chlorophenyl)methoxy]-5-(4,4,5,5-tetramethyl-1,3,2-dioxaborolan-2-yl)pyridin-2-amine

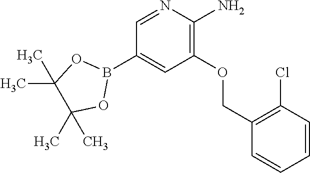 [SMILES: CC1(C)OB(c2cnc(N)c(OCc3ccccc3Cl)c2)OC1(C)C]

Intermediate 210 was prepared in analogy to Intermediate 170 using 5-bromo-3-[(2-chlorophenyl)methoxy]pyridin-2-amine (270 mg, 861 μmol).

LC-MS (method 1): Rt=1.29 min; MS (ESIpos): m/z=361 [M+H]⁺

### Intermediate 211

### 3-[(pyridin-2-yl)methoxy]-5-(4,4,5,5-tetramethyl-1,3,2-dioxaborolan-2-yl)pyridin-2-amine

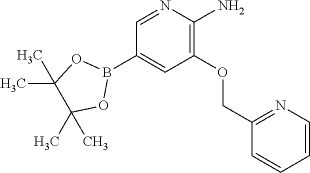 [SMILES: CC1(C)OB(c2cnc(N)c(OCc3ccccn3)c2)OC1(C)C]

Intermediate 211 was prepared in analogy to Intermediate 170 using 5-bromo-3-[(pyridin-2-yl)methoxy]pyridin-2-amine (438 mg, 1.56 mmol).

LC-MS (method 1): Rt=0.79 min; MS (ESIpos): m/z=328 [M+H]⁺

### Intermediate 212

### 3-[(2-chloro-5-fluorophenyl)methoxy]-5-(4,4,5,5-tetramethyl-1,3,2-dioxaborolan-2-yl)pyridin-2-amine

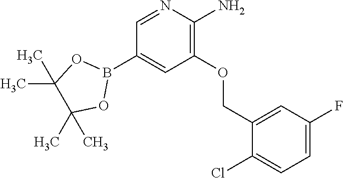 [SMILES: CC1(C)OB(c2cnc(N)c(OCc3cc(F)ccc3Cl)c2)OC1(C)C]

Intermediate 212 was prepared in analogy to Intermediate 170 using 5-bromo-3-[(2-chloro-5-fluorophenyl)methoxy]pyridin-2-amine (480 mg, 1.45 mmol).

LC-MS (method 1): Rt=1.26 min; MS (ESIpos): m/z=379 [M+H]⁺

### Intermediate 213

### 3-[(pyridazin-3-yl)methoxy]-5-(4,4,5,5-tetramethyl-1,3,2-dioxaborolan-2-yl)pyridin-2-amine

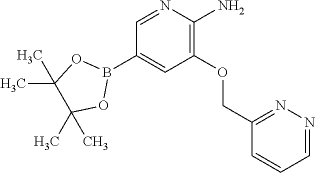 [SMILES: CC1(C)OB(c2cnc(N)c(OCc3cccnn3)c2)OC1(C)C]

Intermediate 213 was prepared in analogy to Intermediate 170 using 5-bromo-3-[(pyridazin-3-yl)methoxy]pyridin-2-amine (180 mg, 640 μmol).

LC-MS (method 1): Rt=0.54 min; MS (ESIpos): m/z=329 [M+H]⁺

### Intermediate 214

### 3-[(1S)-1-(pyridin-2-yl)ethoxy]-5-(4,4,5,5-tetramethyl-1,3,2-dioxaborolan-2-yl)pyridin-2-amine

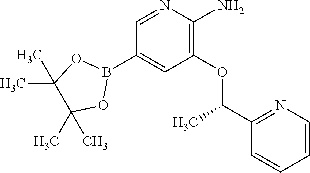 [SMILES: C[C@H](Oc1cc(B2OC(C)(C)C(C)(C)O2)cnc1N)c1ccccn1]

Intermediate 214 was prepared in analogy to Intermediate 170 using 5-bromo-3-[(1S)-1-(pyridin-2-yl)ethoxy]pyridin-2-amine (457 mg, 79% purity, 1.23 mmol).

LC-MS (method 1): Rₜ=0.86 min; MS (ESIpos): m/z=342 [M+H]⁺

### Intermediate 215

### {6-amino-5-[(3-methylphenyl)methoxy]pyridin-3-yl}boronic acid

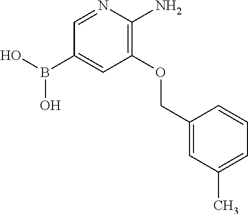 [SMILES: Cc1cccc(COc2cc(B(O)O)cnc2N)c1]

Intermediate 215 was prepared in analogy to Intermediate 170 using 5-bromo-3-[(3-methylphenyl)methoxy]pyridin-2-amine (30.0 mg, 102 μmol).

LC-MS (method 1): Rₜ=0.74 min; MS (ESIpos): m/z=259 [M+H]⁺

¹H-NMR (400 MHz, DMSO-d6) δ[ppm]: 1.066 (0.74), 1.156 (9.51), 1.165 (16.00), 1.261 (1.16), 1.293 (0.46), 3.566 (6.30).

### Intermediate 216

### 3-[(1-methyl-1H-pyrazol-5-yl)methoxy]-5-(4,4,5,5-tetramethyl-1,3,2-dioxaborolan-2-yl)pyridin-2-amine

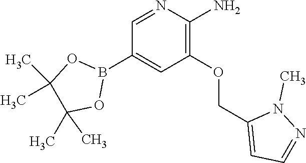 [SMILES: Cn1nccc1COc1cc(B2OC(C)(C)C(C)(C)O2)cnc1N]

Intermediate 216 was prepared in analogy to Intermediate 170 using 5-bromo-3-[(1-methyl-1H-pyrazol-5-yl)methoxy]pyridin-2-amine (230 mg, 812 μmol).

LC-MS (method 1): Rₜ=0.76 min; MS (ESIpos): m/z=331 [M+H]⁺

¹H-NMR (400 MHz, DMSO-d6) δ[ppm]: 1.066 (5.65), 1.156 (2.20), 1.165 (16.00), 1.172 (0.46), 1.272 (1.68), 3.566 (6.36), 3.846 (1.83), 3.939 (0.86).

### Intermediate 217

### 3-[(1S)-1-(1-methyl-1H-pyrazol-3-yl)ethoxy]-5-(4,4,5,5-tetramethyl-1,3,2-dioxaborolan-2-yl)pyridin-2-amine

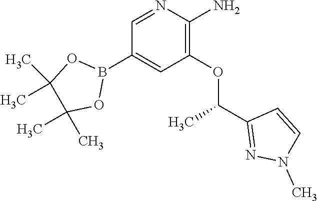 [SMILES: C[C@H](Oc1cc(B2OC(C)(C)C(C)(C)O2)cnc1N)c1ccn(C)n1]

Intermediate 217 was prepared in analogy to Intermediate 170 using 5-bromo-3-[(1S)-1-(1-methyl-1H-pyrazol-3-yl)ethoxy]pyridin-2-amine (149 mg, 501 μmol).

LC-MS (method 1): Rt=0.84 min; MS (ESIpos): m/z=345 [M+H]⁺

### Intermediate 218

### 3-[(1R)-1-(1-methyl-1H-pyrazol-3-yl)ethoxy]-5-(4,4,5,5-tetramethyl-1,3,2-dioxaborolan-2-yl)pyridin-2-amine

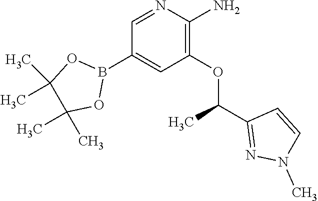 [SMILES: C[C@@H](Oc1cc(B2OC(C)(C)C(C)(C)O2)cnc1N)c1ccn(C)n1]

Intermediate 218 was prepared in analogy to Intermediate 170 using 5-bromo-3-[(1R)-1-(1-methyl-1H-pyrazol-3-yl)ethoxy]pyridin-2-amine (136 mg, 458 μmol).

LC-MS (method 1): Rt=0.85 min; MS (ESIpos): m/z=345 [M+H]⁺

### Intermediate 219

### 1′-methyl-5-(4,4,5,5-tetramethyl-1,3,2-dioxaborolan-2-yl)-1′,2′,3′,6′-tetrahydro[3,4′-bipyridin]-2-amine

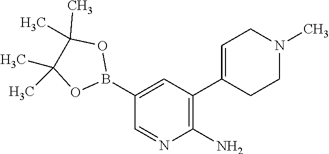

Intermediate 219 was prepared in analogy to Intermediate 170 using 5-bromo-1′-methyl-1′,2′,3′,6′-tetrahydro[3,4′-bipyridin]-2-amine (168 mg, 626 μmol).

LC-MS (method 1): Rₜ=0.95 min; MS (ESIpos): m/z=316 [M+H]⁺

### Intermediate 220

### 3-[(1S)-1-phenylethoxy]-5-(4,4,5,5-tetramethyl-1,3,2-dioxaborolan-2-yl)pyridin-2-amine

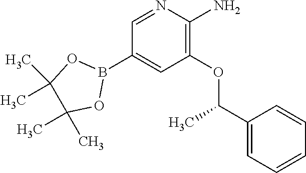 [SMILES: C[C@H](Oc1cc(B2OC(C)(C)C(C)(C)O2)cnc1N)c1ccccc1]

Intermediate 220 was prepared in analogy to Intermediate 170 using 5-bromo-3-[(1S)-1-phenylethoxy]pyridin-2-amine (160 mg, 90% purity, 491 μmol).

LC-MS (method 1): Rt=1.22 min; MS (ESIpos): m/z=341 [M+H]⁺

### Intermediate 221

### 3-[(1R)-1-phenylethoxy]-5-(4,4,5,5-tetramethyl-1,3,2-dioxaborolan-2-yl)pyridin-2-amine

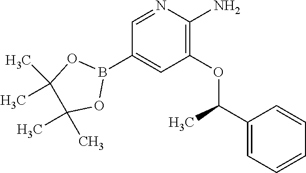 [SMILES: C[C@@H](Oc1cc(B2OC(C)(C)C(C)(C)O2)cnc1N)c1ccccc1]

Intermediate 221 was prepared in analogy to Intermediate 170 using 5-bromo-3-[(1R)-1-phenylethoxy]pyridin-2-amine (160 mg, 90% purity, 491 μmol).

1H-NMR (400 MHz, DMSO-d6) δ[ppm]: 1.066 (16.00), 1.156 (4.55), 1.164 (1.32), 1.172 (0.27), 1.213 (1.79), 1.217 (1.81), 1.275 (0.59), 1.292 (0.76), 1.520 (0.51), 1.536 (0.52), 1.548 (0.19), 1.564 (0.18), 1.987 (0.36), 2.518 (0.35), 2.523 (0.24), 3.566 (6.41), 3.940 (1.44), 6.960 (0.20), 6.963 (0.20), 7.252 (0.17), 7.319 (0.19), 7.334 (0.19), 7.338 (0.32), 7.356 (0.18), 7.436 (0.32), 7.454 (0.21).

### Intermediate 222

### [6-amino-5-(difluoromethyl)pyridin-3-yl]boronic acid

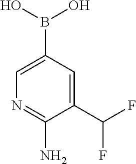 [SMILES: Nc1ncc(B(O)O)cc1C(F)F]

Intermediate 222 was prepared in analogy to Intermediate 170 using 5-bromo-3-(difluoromethyl)pyridin-2-amine (545 mg, 2.44 mmol).

1H-NMR (400 MHz, DMSO-d6) δ[ppm]: 1.065 (1.00), 1.156 (16.00), 1.164 (9.80), 1.267 (6.85), 1.292 (0.78), 1.899 (0.52), 3.565 (3.03), 6.674 (0.44), 7.008 (0.29), 7.782 (0.30), 7.943 (0.33), 8.289 (0.28).

### Intermediate 223

### {6-amino-5-[(cyclohexylmethyl)sulfanyl]pyridin-3-yl}boronic acid

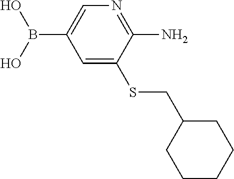 [SMILES: Nc1ncc(B(O)O)cc1SCC1CCCCC1]

5-bromo-3-[(cyclohexylmethyl)sulfanyl]pyridin-2-amine (324 mg, 1.08 mmol), 4,4,4′,4′,5,5,5′,5′-octamethyl-2,2′-bi-1,3,2-dioxaborolane (300 mg, 1.18 mmol), Pd(dppf)Cl2*DCM (317 mg, 3.23 mmol) and KOAc (317 mg, 3.2 mmol) were stirred in degassed 1,4-dioxane (5.5 mL) for 2 h at 100° C. The mixture was filtered and the filtrate was concentrated under reduced pressure to give 731 mg of the title compound, which was used without further purification.

¹H-NMR (400 MHz, DMSO-d6): δ [ppm]=0.90-1.03 (m, 2H), 1.08-1.20 (m, 15H), 1.31-1.40 (m, 2H), 1.54-1.62 (m, 1H), 1.62-1.70 (m, 2H), 1.76-1.84 (m, 2H), 2.65 (d, 2H), 6.47 (s, 2H), 7.63 (d, 1H), 7.94 (s, 1H), 8.11 (d, 1H).

LC-MS (method 1): Rt=0.97 min; MS (ESIpos): m/z=267 [M+H]⁺

### Intermediate 224

### 3-(benzylsulfanyl)-5-(4,4,5,5-tetramethyl-1,3,2-dioxaborolan-2-yl)pyridin-2-amine

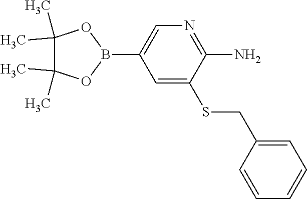 [SMILES: CC1(C)OB(c2cnc(N)c(SCc3ccccc3)c2)OC1(C)C]

Intermediate 224 was prepared in analogy to Intermediate 223 using 3-(benzylsulfanyl)-5-bromopyridin-2-amine (112 mg, 379 μmol).

1H-NMR (400 MHz, DMSO-d6): δ [ppm]=1.24 (s, 12H), 3.98 (s, 2H), 6.55 (s, 2H), 7.18-7.29 (m, 5H), 7.48 (d, 1H), 7.94 (s, 1H), 8.11 (d, 1H).

LC-MS (method 1): Rt=1.25 min; MS (ESIpos): m/z=343 [M+H]⁺

### Intermediate 225

### (rac)-(6-amino-5-{[1-cyclohexylethyl]sulfanyl}pyridin-3-yl)boronic acid

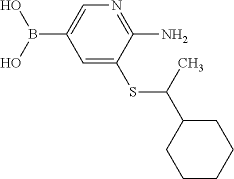 [SMILES: CC(Sc1cc(B(O)O)cnc1N)C1CCCCC1]

Intermediate 225 was prepared in analogy to Intermediate 223 using (rac)-5-bromo-3-{[1-cyclohexylethyl]sulfanyl}pyridin-2-amine (64.0 mg, 203 μmol).

LC-MS (method 1): Rt=1.06 min; MS (ESIpos): m/z=281 [M+H]⁺

¹H-NMR (400 MHz, DMSO-d6) δ[ppm]: 1.066 (1.86), 1.077 (0.98), 1.094 (1.09), 1.114 (0.48), 1.133 (0.42), 1.156 (16.00), 1.258 (4.86), 3.565 (12.80), 7.657 (0.44), 7.661 (0.46), 7.941 (1.28), 8.146 (0.43), 8.150 (0.43).

### Intermediate 226

### {6-amino-5-[(1R)-1-(pyridin-3-yl)ethoxy]pyridin-3-yl}boronic acid

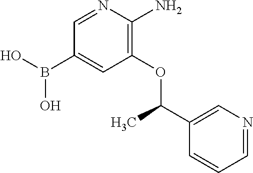 [SMILES: C[C@@H](Oc1cc(B(O)O)cnc1N)c1cccnc1]

Intermediate 226 was prepared in analogy to Intermediate 223 using 5-bromo-3-[(1R)-1-(pyridin-3-yl)ethoxy]pyridin-2-amine (76.0 mg, 258 μmol).

¹H-NMR (400 MHz, DMSO-d6): δ [ppm]=1.23 (s, 12H), 1.55 (d, 3H), 5.66 (q, 1H), 6.27 (s, 2H), 7.05 (d, 1H), 7.38 (dd, 1H), 7.78 (d, 1H), 7.91 (dt, 1H), 8.48 (dd, 1H), 8.69 (d, 1H).

LC-MS (method 1): Rt=0.49 min; MS (ESIpos): m/z=260 [M+H]⁺

### Intermediate 227

### {6-amino-5-[(1S)-1-(pyridin-3-yl)ethoxy]pyridin-3-yl}boronic acid

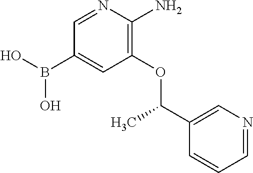 [SMILES: C[C@H](Oc1cc(B(O)O)cnc1N)c1cccnc1]

Intermediate 227 was prepared in analogy to Intermediate 223 using 5-bromo-3-[(1S)-1-(pyridin-3-yl)ethoxy]pyridin-2-amine (110 mg, 374 μmol).

¹H-NMR (400 MHz, DMSO-d6): δ [ppm]=1.23 (s, 12H), 1.55 (d, 3H), 5.66 (q, 1H), 6.27 (s, 2H), 7.05 (d, 1H), 7.35-7.41 (m, 1H), 7.78 (d, 1H), 7.91 (dt, 1H), 8.48 (dd, 1H), 8.69 (d, 1H).

LC-MS (method 1): Rt=0.49 min; MS (ESIpos): m/z=260 [M+H]⁺

### Intermediate 228

### (6-amino-5-phenylpyridin-3-yl)boronic acid

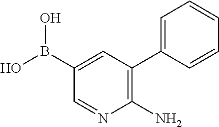 [SMILES: Nc1ncc(B(O)O)cc1-c1ccccc1]

Intermediate 228 was prepared in analogy to Intermediate 223 using 5-bromo-3-phenylpyridin-2-amine (175 mg, 702 μmol).

LC-MS (method 2): Rt=0.52 min; MS (ESIpos): m/z=215 [M+H]⁺

### Intermediate 229

### [6-amino-5-(4-methylphenyl)pyridin-3-yl]boronic acid

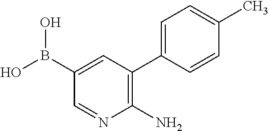 [SMILES: Cc1ccc(-c2cc(B(O)O)cnc2N)cc1]

Intermediate 229 was prepared in analogy to Intermediate 223 using 5-bromo-3-(4-methylphenyl)pyridin-2-amine (190 mg, 722 μmol).

LC-MS (method 1): Rt=0.65 min; MS (ESIpos): m/z=229 [M+H]⁺

### Intermediate 230

### [6-amino-5-(2-methylphenyl)pyridin-3-yl]boronic acid

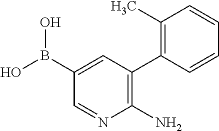 [SMILES: Cc1ccccc1-c1cc(B(O)O)cnc1N]

Intermediate 230 was prepared in analogy to Intermediate 223 using 5-bromo-3-(2-methylphenyl)pyridin-2-amine (191 mg, 726 μmol).

LC-MS (method 2): Rt=0.61 min; MS (ESIpos): m/z=229 [M+H]⁺

### Intermediate 231

### [6-amino-5-(3-methylphenyl)pyridin-3-yl]boronic acid

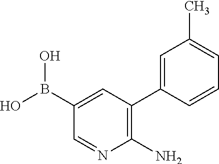 [SMILES: Cc1cccc(-c2cc(B(O)O)cnc2N)c1]

Intermediate 231 was prepared in analogy to Intermediate 223 using 5-bromo-3-(3-methylphenyl)pyridin-2-amine (187 mg, 711 μmol).

LC-MS (method 2): Rt=0.66 min; MS (ESIpos): m/z=229 [M+H]⁺

### Intermediate 232

### 5-(4,4,5,5-tetramethyl-1,3,2-dioxaborolan-2-yl)[3,3′-bipyridin]-2-amine

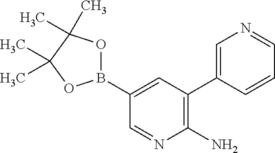 [SMILES: CC1(C)OB(c2cnc(N)c(-c3cccnc3)c2)OC1(C)C]

Intermediate 232 was prepared in analogy to Intermediate 223 using 5-bromo[3,3′-bipyridin]-2-amine (225 mg, 900 μmol).

1H-NMR (400 MHz, DMSO-d6) δ[ppm]: 1.066 (3.47), 1.156 (15.42), 1.164 (11.69), 1.263 (4.98), 1.292 (0.70), 1.311 (0.22), 1.905 (0.78), 2.518 (0.75), 2.523 (0.52), 3.566 (16.00), 3.938 (0.45), 6.224 (0.34), 7.425 (0.35), 7.429 (0.36), 7.939 (0.73), 8.228 (0.38), 8.233 (0.39), 8.557 (0.17), 8.561 (0.19), 8.569 (0.18), 8.573 (0.17), 8.586 (0.22), 8.590 (0.21).

### Intermediate 233

### (2-amino[3,4′-bipyridin]-5-yl)boronic acid

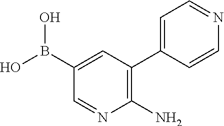 [SMILES: Nc1ncc(B(O)O)cc1-c1ccncc1]

Intermediate 233 was prepared in analogy to Intermediate 223 using 5-bromo[3,4′-bipyridin]-2-amine (190 mg, 760 μmol).

LC-MS (method 1): Rt=0.67 min; MS (ESIpos): m/z=216 [M+H]⁺

### Intermediate 234

### (rac)-3-[1-(5-chloro-3-fluoropyridin-2-yl)ethoxy]-5-(4,4,5,5-tetramethyl-1,3,2-dioxaborolan-2-yl)pyridin-2-amine

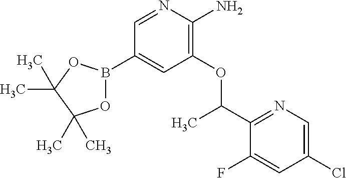 [SMILES: CC(Oc1cc(B2OC(C)(C)C(C)(C)O2)cnc1N)c1ncc(Cl)cc1F]

Intermediate 234 was prepared in analogy to Intermediate 223 using (rac)-5-bromo-3-[1-(5-chloro-3-fluoropyridin-2-yl)ethoxy]pyridin-2-amine (300 mg, 866 μmol).

LC-MS (method 1): Rt=1.12 min; MS (ESIpos): m/z=394 [M+H]⁺

### Intermediate 235

### (rac)-tert-butyl-2′-{6-amino-5-[(trifluoromethyl)sulfanyl]pyridin-3-yl}-5′,6′-dihydrospiro[pyrrolidine-3,4′-pyrrolo[1,2-b]pyrazole]-1-carboxylate

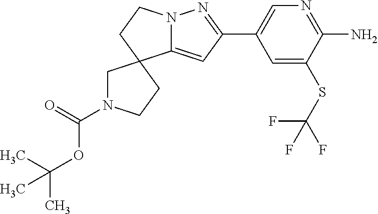 [SMILES: CC(C)(C)OC(=O)N1CCC2(CCn3nc(-c4cnc(N)c(SC(F)(F)F)c4)cc32)C1]

(rac)-tert-butyl-2′-[(trifluoromethanesulfonyl)oxy]-5′,6′-dihydrospiro[pyrrolidine-3,4′-pyrrolo[1,2-b]pyrazole]-1-carboxylate (1.61 g, 3.92 mmol) was solubilised in 1,4-dioxane (33 mL) under nitrogen, {6-amino-5-[(trifluoromethyl)sulfanyl]pyridin-3-yl}boronic acid (1.40 g, 5.88 mmol), K3PO4 (23.5 mL, 0.50 M, 11.76 mmol) and XPhos Pd G2 (309 mg, 392 μmol) were added and the mixture was stirred overnight at 100° C. The mixture was diluted with ethyl acetate and washed with brine. The organic layer was dried over a silicone filter and evaporated. The residue was purified by flash chromatography and preparative HPLC to give 1.20 g (67% yield) of the title compound.

LC-MS (method 1): Rₜ=1.27 min; MS (ESIpos): m/z=456 [M+H]⁺

¹H-NMR (400 MHz, DMSO-d6) δ[ppm]: 1.395 (16.00), 1.433 (13.07), 2.034 (1.64), 2.047 (1.52), 2.074 (1.50), 2.084 (1.38), 3.283 (0.48), 3.408 (6.28), 3.493 (0.69), 3.511 (1.27), 3.537 (0.80), 4.090 (2.05), 4.173 (2.19), 4.180 (2.13), 5.766 (0.49), 6.432 (1.68), 6.457 (2.12), 6.737 (5.20), 7.538 (0.74), 7.645 (0.51), 8.050 (2.87), 8.523 (3.46), 8.529 (3.50).

### Intermediate 236

### (rac)-tert-butyl-2′-[6-amino-5-(trifluoromethoxy)pyridin-3-yl]-5′,6′-dihydrospiro[pyrrolidine-3,4′-pyrrolo[1,2-b]pyrazole]-1-carboxylate

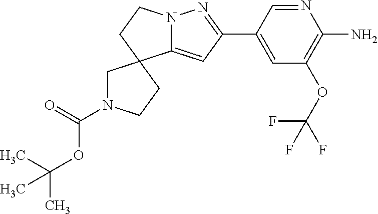 [SMILES: CC(C)(C)OC(=O)N1CCC2(CCn3nc(-c4cnc(N)c(OC(F)(F)F)c4)cc32)C1]

Intermediate 236 was prepared in analogy to Intermediate 235 using (rac)-tert-butyl-2′-[(trifluoromethanesulfonyl)oxy]-5′,6′-dihydrospiro[pyrrolidine-3,4′-pyrrolo[1,2-b]pyrazole]-1-carboxylate (890 mg, 2.16 mmol) and [6-amino-5-(trifluoromethoxy)pyridin-3-yl]boronic acid (1.20 g, 60% purity, 3.24 mmol).

LC-MS (method 1): Rt=1.21 min; MS (ESIpos): m/z=440 [M+H]⁺

1H-NMR (400 MHz, DMSO-d6) δ[ppm]: 1.066 (16.00), 1.154 (0.40), 1.173 (0.82), 1.190 (0.43), 1.395 (0.82), 1.432 (0.65), 1.988 (1.55), 2.518 (0.22), 2.523 (0.18), 3.408 (0.32), 3.937 (2.91), 4.017 (0.36), 4.035 (0.35), 6.495 (0.28), 8.330 (0.26), 8.334 (0.25).

### Intermediate 237

### (rac)-tert-butyl-2′-[6-amino-5-(difluoromethoxy)pyridin-3-yl]-5′,6′-dihydrospiro[pyrrolidine-3,4′-pyrrolo[1,2-b]pyrazole]-1-carboxylate

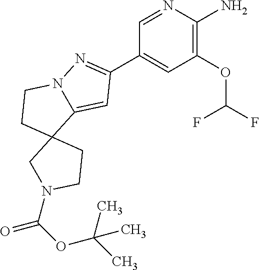 [SMILES: CC(C)(C)OC(=O)N1CCC2(CCn3nc(-c4cnc(N)c(OC(F)F)c4)cc32)C1]

Intermediate 237 was prepared in analogy to Intermediate 235 using (rac)-tert-butyl-2′-[(trifluoromethanesulfonyl)oxy]-5′,6′-dihydrospiro[pyrrolidine-3,4′-pyrrolo[1,2-b]pyrazole]-1-carboxylate (1.50 g, 3.64 mmol) and 3-(difluoromethoxy)-5-(4,4,5,5-tetramethyl-1,3,2-dioxaborolan-2-yl)pyridin-2-amine (2.50 g, 48% purity, 4.18 mmol).

LC-MS (method 1): Rt=1.12 min; MS (ESIpos): m/z=422 [M+H]⁺

1H-NMR (400 MHz, DMSO-d6): δ [ppm]=1.41 (d, 9H), 1.95-2.12 (m, 3H), 2.53-2.59 (m, 2H), 3.37-3.46 (m, 3H), 3.47-3.60 (m, 1H), 4.12-4.24 (m, 2H), 6.16 (s, 2H), 6.39 (d, 1H), 7.18 (t, 1H), 7.62 (s, 1H), 8.20 (s, 1H).

### Intermediate 238

### tert-butyl-2′-{6-amino-5-[(1R)-1-(pyridin-2-yl)ethoxy]pyridin-3-yl}-5′,6′-dihydrospiro[pyrrolidine-3,4′-pyrrolo[1,2-b]pyrazole]-1-carboxylate

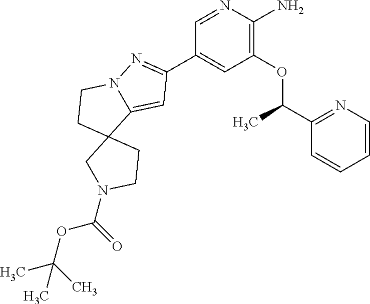 [SMILES: C[C@@H](Oc1cc(-c2cc3n(n2)CCC32CCN(C(=O)OC(C)(C)C)C2)cnc1N)c1ccccn1]

Intermediate 238 was prepared in analogy to Intermediate 235 using 3-[(1R)-1-(pyridin-2-yl)ethoxy]-5-(4,4,5,5-tetramethyl-1,3,2-dioxaborolan-2-yl)pyridin-2-amine (312 mg, 914 μmol) and (rac)-tert-butyl-2′-[(trifluoromethanesulfonyl)oxy]-5′,6′-dihydrospiro[pyrrolidine-3,4′-pyrrolo[1,2-b]pyrazole]-1-carboxylate (451 mg, 1.10 mmol).

LC-MS (method 1): Rₜ=1.11 min; MS (ESIpos): m/z=478 [M+H]⁺

¹H-NMR (400 MHz, DMSO-d6) δ[ppm]: 0.798 (0.54), 0.815 (0.54), 0.822 (0.57), 0.905 (0.72), 1.035 (5.63), 1.053 (12.08), 1.066 (13.92), 1.070 (5.69), 1.388 (13.53), 1.392 (13.86), 1.430 (16.00), 1.589 (0.99), 1.609 (9.82), 1.625 (9.97), 2.016 (1.42), 2.322 (1.11), 2.327 (1.66), 2.332 (1.24), 2.518 (7.50), 2.523 (5.03), 2.660 (0.54), 2.665 (1.21), 2.669 (1.72), 2.673 (1.21), 3.374 (5.15), 3.380 (4.61), 3.405 (1.60), 3.418 (1.30), 3.422 (2.47), 3.435 (2.32), 3.440 (2.23), 3.452 (2.20), 3.457 (0.81), 3.463 (0.72), 3.469 (0.96), 3.481 (1.21), 3.497 (0.84), 3.508 (0.75), 3.525 (0.45), 3.938 (1.96), 4.100 (1.18), 4.109 (1.48), 4.119 (2.20), 4.125 (2.26), 4.135 (1.48), 4.143 (1.33), 4.343 (1.21), 4.355 (2.38), 4.368 (1.15), 5.507 (1.21), 5.522 (1.24), 5.903 (3.59), 6.224 (2.02), 6.236 (1.72), 6.242 (1.48), 7.208 (2.86), 7.275 (1.18), 7.278 (1.02), 7.290 (1.69), 7.305 (1.36), 7.493 (2.23), 7.511 (2.56), 7.767 (1.15), 7.787 (2.05), 7.806 (0.93), 7.864 (3.95), 7.868 (4.01), 8.539 (1.81), 8.546 (1.84), 8.551 (1.75).

### Intermediate 239

### tert-butyl-2′-{6-amino-5-[(1S)-1-(pyridin-2-yl)ethoxy]pyridin-3-yl}-5′,6′-dihydrospiro[pyrrolidine-3,4′-pyrrolo[1,2-b]pyrazole]-1-carboxylate

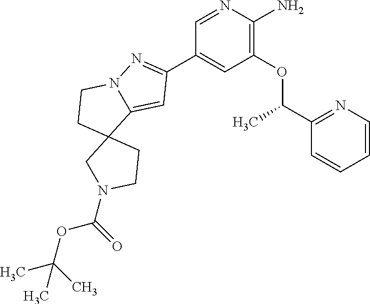 [SMILES: C[C@H](Oc1cc(-c2cc3n(n2)CCC32CCN(C(=O)OC(C)(C)C)C2)cnc1N)c1ccccn1]

Intermediate 239 was prepared in analogy to Intermediate 235 using 3-[(1S)-1-(pyridin-2-yl)ethoxy]-5-(4,4,5,5-tetramethyl-1,3,2-dioxaborolan-2-yl)pyridin-2-amine (360 mg, 1.06 mmol) and (rac)-tert-butyl-2′-[(trifluoromethanesulfonyl)oxy]-5′,6′-dihydrospiro[pyrrolidine-3,4′-pyrrolo[1,2-b]pyrazole]-1-carboxylate (521 mg, 1.27 mmol).

LC-MS (method 1): Rₜ=1.11 min; MS (ESIpos): m/z=478 [M+H]⁺

¹H-NMR (400 MHz, DMSO-d6) δ[ppm]: 1.035 (7.56), 1.053 (16.00), 1.066 (1.32), 1.070 (7.02), 1.388 (10.77), 1.392 (11.11), 1.430 (12.71), 1.594 (1.78), 1.611 (8.85), 1.627 (7.76), 2.005 (1.09), 2.015 (1.14), 2.322 (0.58), 2.327 (0.86), 2.332 (0.63), 2.518 (4.60), 2.523 (3.03), 2.665 (0.61), 2.669 (0.89), 2.673 (0.65), 3.375 (4.17), 3.381 (3.77), 3.405 (1.34), 3.417 (1.05), 3.423 (1.80), 3.435 (1.69), 3.440 (1.58), 3.452 (1.51), 3.457 (0.74), 3.463 (0.69), 3.469 (0.74), 3.480 (0.98), 3.496 (0.71), 3.508 (0.61), 4.102 (0.97), 4.110 (1.18), 4.121 (1.72), 4.126 (1.83), 4.136 (1.18), 4.144 (1.09), 4.343 (0.66), 4.356 (1.26), 4.369 (0.66), 5.514 (1.00), 5.530 (1.03), 5.955 (1.77), 6.228 (1.63), 6.242 (1.38), 6.247 (1.20), 7.218 (2.31), 7.276 (0.91), 7.278 (0.92), 7.290 (1.49), 7.297 (1.05), 7.306 (1.05), 7.309 (0.97), 7.492 (1.75), 7.494 (1.75), 7.512 (2.04), 7.514 (2.00), 7.766 (1.03), 7.786 (1.81), 7.805 (0.91), 7.863 (3.32), 7.867 (3.37), 8.541 (1.54), 8.546 (1.61), 8.551 (1.46).

### Intermediate 240

### tert-butyl-2′-{6-amino-5-[(1S)-1-(pyridin-3-yl)ethoxy]pyridin-3-yl}-5′,6′-dihydrospiro[pyrrolidine-3,4′-pyrrolo[1,2-b]pyrazole]-1-carboxylate

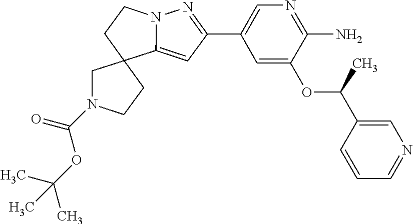 [SMILES: C[C@H](Oc1cc(-c2cc3n(n2)CCC32CCN(C(=O)OC(C)(C)C)C2)cnc1N)c1cccnc1]

Intermediate 240 was prepared in analogy to Intermediate 235 using (rac)-tert-butyl-2′-[(trifluoromethanesulfonyl)oxy]-5′,6′-dihydrospiro[pyrrolidine-3,4′-pyrrolo[1,2-b]pyrazole]-1-carboxylate (540 mg, 1.31 mmol) and 3-[(1S)-1-(pyridin-3-yl)ethoxy]-5-(4,4,5,5-tetramethyl-1,3,2-dioxaborolan-2-yl)pyridin-2-amine (1.60 g, 31% purity, 1.44 mmol).

¹H-NMR (400 MHz, DMSO-d6): δ [ppm]=1.37-1.46 (m, 9H), 1.60 (d, 3H), 1.95-2.09 (m, 2H), 3.36-3.45 (m, 3H), 3.47-3.55 (m, 1H), 4.08-4.19 (m, 2H), 5.70 (q, 1H), 5.90 (s, 2H), 6.28 (dd, 1H), 7.32 (s, 1H), 7.35-7.40 (m, 1H), 7.87 (d, 1H), 7.89-7.94 (m, 1H), 8.47 (dd, 1H), 8.70 (s, 1H).

LC-MS (method 1): Rt=1.08 min; MS (ESIpos): m/z=478 [M+H]⁺

### Intermediate 241

### (rac)-tert-butyl-2′-{6-amino-5-[(2-fluorophenyl)methoxy]pyridin-3-yl}-5′,6′-dihydrospiro[pyrrolidine-3,4′-pyrrolo[1,2-b]pyrazole]-1-carboxylate

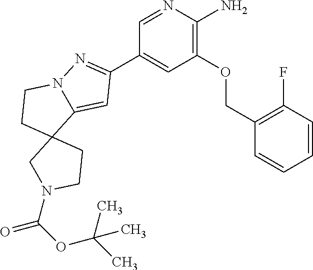 [SMILES: CC(C)(C)OC(=O)N1CCC2(CCn3nc(-c4cnc(N)c(OCc5ccccc5F)c4)cc32)C1]

Intermediate 241 was prepared in analogy to Intermediate 235 using (rac)-tert-butyl-2′-[(trifluoromethanesulfonyl)oxy]-5′,6′-dihydrospiro[pyrrolidine-3,4′-pyrrolo[1,2-b]pyrazole]-1-carboxylate (60.0 mg, 146 μmol) and 3-[(2-fluorophenyl)methoxy]-5-(4,4,5,5-tetramethyl-1,3,2-dioxaborolan-2-yl)pyridin-2-amine (122 mg, 47% purity, 168 μmol).

LC-MS (method 1): Rt=1.25 min; MS (ESIpos): m/z=480 [M+H]⁺

1H-NMR (400 MHz, DMSO-d6): δ [ppm]=1.42 (d, 9H), 1.96-2.13 (m, 2H), 3.37-3.46 (m, 3H), 3.53 (dt, 1H), 4.17 (td, 2H), 5.21 (s, 2H), 5.77 (s, 2H), 6.37 (d, 1H), 7.21-7.31 (m, 2H), 7.38-7.46 (m, 1H), 7.48 (s, 1H), 7.67 (td, 1H), 7.96 (d, 1H).

### Intermediate 242

### (rac)-tert-butyl-2′-{6-amino-5-[(3-fluorophenyl)methoxy]pyridin-3-yl}-5′,6′-dihydrospiro[pyrrolidine-3,4′-pyrrolo[1,2-b]pyrazole]-1-carboxylate

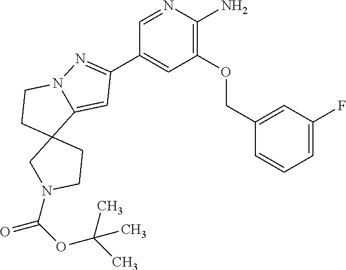 [SMILES: CC(C)(C)OC(=O)N1CCC2(CCn3nc(-c4cnc(N)c(OCc5cccc(F)c5)c4)cc32)C1]

Intermediate 242 was prepared in analogy to Intermediate 235 using (rac)-tert-butyl-2′-[(trifluoromethanesulfonyl)oxy]-5′,6′-dihydrospiro[pyrrolidine-3,4′-pyrrolo[1,2-b]pyrazole]-1-carboxylate (415 mg, 1.01 mmol) and 3-[(3-fluorophenyl)methoxy]-5-(4,4,5,5-tetramethyl-1,3,2-dioxaborolan-2-yl)pyridin-2-amine (938 mg, 43% purity, 1.16 mmol).

LC-MS (method 1): Rt=1.24 min; MS (ESIpos): m/z=480 [M+H]⁺

1H-NMR (400 MHz, DMSO-d6) δ[ppm]: 1.066 (1.99), 1.154 (4.82), 1.172 (9.61), 1.190 (4.87), 1.398 (10.46), 1.435 (8.48), 1.988 (16.00), 2.039 (0.88), 2.047 (0.89), 2.068 (0.62), 2.327 (0.44), 2.669 (0.47), 3.369 (0.40), 3.410 (3.92), 3.426 (0.89), 3.509 (0.73), 3.524 (0.50), 3.536 (0.47), 3.999 (1.24), 4.017 (3.69), 4.035 (3.62), 4.053 (1.22), 4.138 (0.69), 4.147 (0.84), 4.156 (1.31), 4.163 (1.38), 4.173 (0.95), 4.181 (0.82), 5.204 (4.87), 5.251 (0.96), 5.808 (0.59), 5.876 (2.95), 6.341 (1.07), 6.364 (1.42), 7.132 (0.47), 7.153 (0.98), 7.169 (0.52), 7.173 (0.58), 7.350 (1.04), 7.369 (1.73), 7.410 (1.46), 7.426 (3.52), 7.430 (3.89), 7.445 (1.44), 7.450 (1.01), 7.466 (0.59), 7.773 (0.46), 7.778 (0.49), 7.939 (2.34), 7.943 (2.52).

### Intermediate 243

### (rac)-tert-butyl-2′-{6-amino-5-[(2,6-dichlorophenyl)methoxy]pyridin-3-yl}-5′,6′-dihydrospiro[pyrrolidine-3,4′-pyrrolo[1,2-b]pyrazole]-1-carboxylate

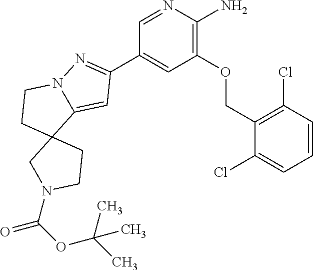 [SMILES: CC(C)(C)OC(=O)N1CCC2(CCn3nc(-c4cnc(N)c(OCc5c(Cl)cccc5Cl)c4)cc32)C1]

Intermediate 243 was prepared in analogy to Intermediate 235 using (rac)-tert-butyl-2′-[(trifluoromethanesulfonyl)oxy]-5′,6′-dihydrospiro[pyrrolidine-3,4′-pyrrolo[1,2-b]pyrazole]-1-carboxylate (404 mg, 981 μmol) and 3-[(2,6-dichlorophenyl)methoxy]-5-(4,4,5,5-tetramethyl-1,3,2-dioxaborolan-2-yl)pyridin-2-amine (1.33 g, 33% purity, 1.13 mmol).

LC-MS (method 1): Rt=1.34 min; MS (ESIpos): m/z=530 [M+H]⁺

### Intermediate 244

### (rac)-tert-butyl-2′-{6-amino-5-[(2-chlorophenyl)methoxy]pyridin-3-yl}-5′,6′-dihydrospiro[pyrrolidine-3,4′-pyrrolo[1,2-b]pyrazole]-1-carboxylate

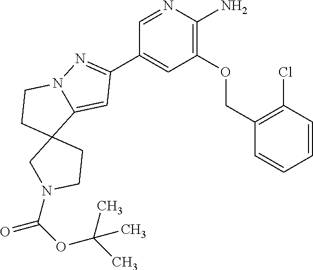 [SMILES: CC(C)(C)OC(=O)N1CCC2(CCn3nc(-c4cnc(N)c(OCc5ccccc5Cl)c4)cc32)C1]

Intermediate 244 was prepared in analogy to Intermediate 235 using (rac)-tert-butyl-2′-[(trifluoromethanesulfonyl)oxy]-5′,6′-dihydrospiro[pyrrolidine-3,4′-pyrrolo[1,2-b]pyrazole]-1-carboxylate (307 mg, 747 μmol) and 3-[(2-chlorophenyl)methoxy]-5-(4,4,5,5-tetramethyl-1,3,2-dioxaborolan-2-yl)pyridin-2-amine (632 mg, 49% purity, 859 μmol).

LC-MS (method 1): Rt=1.29 min; MS (ESIpos): m/z=497 [M+H]⁺

### Intermediate 245

### (rac)-tert-butyl-2′-{6-amino-5-[(pyridin-2-yl)methoxy]pyridin-3-yl}-5′,6′-dihydrospiro[pyrrolidine-3,4′-pyrrolo[1,2-b]pyrazole]-1-carboxylate

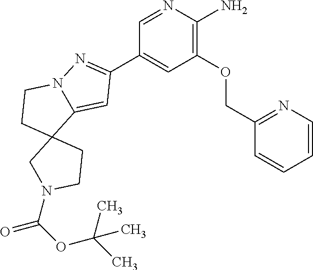 [SMILES: CC(C)(C)OC(=O)N1CCC2(CCn3nc(-c4cnc(N)c(OCc5ccccn5)c4)cc32)C1]

Intermediate 245 was prepared in analogy to Intermediate 235 using (rac)-tert-butyl-2′-[(trifluoromethanesulfonyl)oxy]-5′,6′-dihydrospiro[pyrrolidine-3,4′-pyrrolo[1,2-b]pyrazole]-1-carboxylate (559 mg, 1.36 mmol) and 3-[(pyridin-2-yl)methoxy]-5-(4,4,5,5-tetramethyl-1,3,2-dioxaborolan-2-yl)pyridin-2-amine (950 mg, 54% purity, 1.56 mmol).

LC-MS (method 1): Rt=1.07 min; MS (ESIpos): m/z=463 [M+H]⁺

### Intermediate 246

### (rac)-tert-butyl-2′-{6-amino-5-[(2-chloro-5-fluorophenyl)methoxy]pyridin-3-yl}-5′,6′-dihydrospiro[pyrrolidine-3,4′-pyrrolo[1,2-b]pyrazole]-1-carboxylate

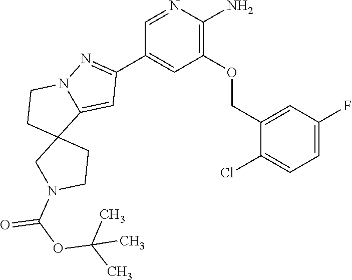 [SMILES: CC(C)(C)OC(=O)N1CCC2(CCn3nc(-c4cnc(N)c(OCc5cc(F)ccc5Cl)c4)cc32)C1]

Intermediate 246 was prepared in analogy to Intermediate 235 using (rac)-tert-butyl-2′-[(trifluoromethanesulfonyl)oxy]-5′,6′-dihydrospiro[pyrrolidine-3,4′-pyrrolo[1,2-b]pyrazole]-1-carboxylate (515 mg, 1.25 mmol) and 3-[(2-chloro-5-fluorophenyl)methoxy]-5-(4,4,5,5-tetramethyl-1,3,2-dioxaborolan-2-yl)pyridin-2-amine (940 mg, 58% purity, 1.44 mmol).

LC-MS (method 1): Rt=1.30 min; MS (ESIpos): m/z=514 [M+H]⁺

### Intermediate 247

### tert-butyl-2′-{6-amino-5-[(1R)-1-(pyridin-3-yl)ethoxy]pyridin-3-yl}-5′,6′-dihydrospiro[pyrrolidine-3,4′-pyrrolo[1,2-b]pyrazole]-1-carboxylate

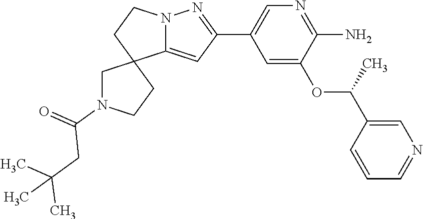 [SMILES: C[C@@H](Oc1cc(-c2cc3n(n2)CCC32CCN(C(=O)CC(C)(C)C)C2)cnc1N)c1cccnc1]

Intermediate 247 was prepared in analogy to Intermediate 235 using (rac)-tert-butyl-2′-[(trifluoromethanesulfonyl)oxy]-5′,6′-dihydrospiro[pyrrolidine-3,4′-pyrrolo[1,2-b]pyrazole]-1-carboxylate (581 mg, 1.41 mmol) and 3-[(1R)-1-(pyridin-3-yl)ethoxy]-5-(4,4,5,5-tetramethyl-1,3,2-dioxaborolan-2-yl)pyridin-2-amine (1.00 g, 53% purity, 1.55 mmol).

1H-NMR (400 MHz, DMSO-d6): δ [ppm]=1.37-1.45 (m, 9H), 1.60 (d, 3H), 1.95-2.09 (m, 2H), 3.36-3.45 (m, 3H), 3.46-3.55 (m, 1H), 4.07-4.19 (m, 2H), 5.70 (q, 1H), 5.90 (s, 2H), 6.28 (dd, 1H), 7.32 (s, 1H), 7.34-7.40 (m, 1H), 7.87 (d, 1H), 7.91 (br d, 1H), 8.46 and 8.47 (2d, 1H), 8.70 (s, 1H).

LC-MS (method 1): Rt=1.05 min; MS (ESIpos): m/z=477 [M+H]⁺

### Intermediate 248

### (rac)-tert-butyl-2′-(2-amino-1′-methyl-1′,2′,3′,6′-tetrahydro[3,4′-bipyridin]-5-yl)-5′,6′-dihydrospiro[pyrrolidine-3,4′-pyrrolo[1,2-b]pyrazole]-1-carboxylate

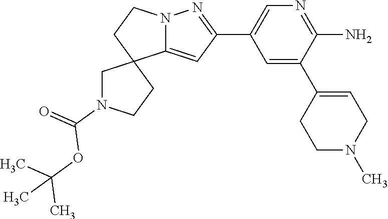 [SMILES: CN1CC=C(c2cc(-c3cc4n(n3)CCC43CCN(C(=O)OC(C)(C)C)C3)cnc2N)CC1]

Intermediate 248 was prepared in analogy to Intermediate 235 using (rac)-tert-butyl-2′-[(trifluoromethanesulfonyl)oxy]-5′,6′-dihydrospiro[pyrrolidine-3,4′-pyrrolo[1,2-b]pyrazole]-1-carboxylate (159 mg, 386 μmol) and 1′-methyl-5-(4,4,5,5-tetramethyl-1,3,2-dioxaborolan-2-yl)-1′,2′,3′,6′-tetrahydro[3,4′-bipyridin]-2-amine (434 mg, 42% purity, 578 μmol).

LC-MS (method 1): Rₜ=1.09 min; MS (ESIpos): m/z=452 [M+H]⁺

¹H-NMR (400 MHz, CHLOROFORM-d) 5 [ppm]: 1.250 (16.00), 1.329 (0.65), 1.474 (1.26), 1.509 (0.86), 2.427 (1.53), 5.309 (0.76).

### Intermediate 249

### tert-butyl-2′-{6-amino-5-[(1R)-1-(pyridin-4-yl)ethoxy]pyridin-3-yl}-5′,6′-dihydrospiro[pyrrolidine-3,4′-pyrrolo[1,2-b]pyrazole]-1-carboxylate

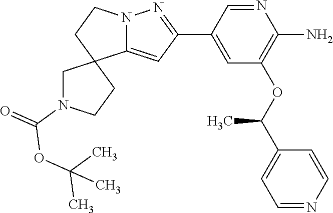 [SMILES: C[C@@H](Oc1cc(-c2cc3n(n2)CCC32CCN(C(=O)OC(C)(C)C)C2)cnc1N)c1ccncc1]

Intermediate 249 was prepared in analogy to Intermediate 235 using (rac)-tert-butyl-2′-[(trifluoromethanesulfonyl)oxy]-5′,6′-dihydrospiro[pyrrolidine-3,4′-pyrrolo[1,2-b]pyrazole]-1-carboxylate (180 mg, 437 μmol) and {6-amino-5-[(1R)-1-(pyridin-4-yl)ethoxy]pyridin-3-yl}boronic acid (170 mg, 656 μmol).

LC-MS (method 1): Rₜ=1.08 min; MS (ESIpos): m/z=477 [M+H]⁺

### Intermediate 250

### tert-butyl-2′-{6-amino-5-[(1S)-1-phenylethoxy]pyridin-3-yl}-5′,6′-dihydrospiro[pyrrolidine-3,4′-pyrrolo[1,2-b]pyrazole]-1-carboxylate

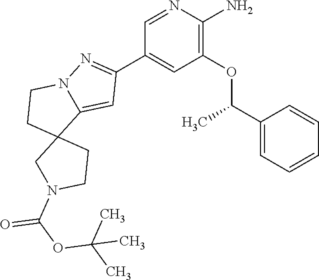 [SMILES: C[C@H](Oc1cc(-c2cc3n(n2)CCC32CCN(C(=O)OC(C)(C)C)C2)cnc1N)c1ccccc1]

Intermediate 250 was prepared in analogy to Intermediate 235 using 3-[(1S)-1-phenylethoxy]-5-(4,4,5,5-tetramethyl-1,3,2-dioxaborolan-2-yl)pyridin-2-amine (200 mg, 60% purity, 353 μmol) and (rac)-tert-butyl-2′-[(trifluoromethanesulfonyl)oxy]-5′,6′-dihydrospiro[pyrrolidine-3,4′-pyrrolo[1,2-b]pyrazole]-1-carboxylate (160 mg, 388 μmol).

LC-MS (method 1): Rt=1.26 min; MS (ESIpos): m/z=476 [M+H]⁺

1H-NMR (400 MHz, DMSO-d6) δ[ppm]: 1.066 (16.00), 1.173 (0.21), 1.389 (2.77), 1.398 (2.89), 1.433 (3.22), 1.563 (2.22), 1.579 (2.19), 1.988 (0.54), 2.017 (0.38), 2.323 (0.26), 2.327 (0.36), 2.332 (0.28), 2.523 (1.32), 2.665 (0.26), 2.669 (0.38), 2.673 (0.27), 3.379 (1.37), 3.405 (0.38), 3.468 (0.17), 3.486 (0.32), 3.500 (0.22), 3.939 (2.84), 4.090 (0.36), 4.102 (0.32), 4.111 (0.39), 4.121 (0.58), 4.127 (0.58), 4.137 (0.38), 4.144 (0.34), 5.568 (0.30), 5.583 (0.30), 5.831 (1.44), 6.222 (0.40), 6.237 (0.39), 6.245 (0.36), 7.230 (0.90), 7.241 (0.51), 7.245 (0.47), 7.259 (0.36), 7.314 (0.47), 7.319 (0.53), 7.333 (0.91), 7.338 (0.89), 7.352 (0.47), 7.357 (0.43), 7.452 (1.21), 7.471 (0.88), 7.837 (1.22), 7.842 (1.22).

### Intermediate 251

### tert-butyl-2′-{6-amino-5-[(1R)-1-phenylethoxy]pyridin-3-yl}-5′,6′-dihydrospiro[pyrrolidine-3,4′-pyrrolo[1,2-b]pyrazole]-1-carboxylate

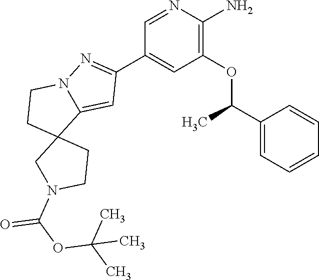 [SMILES: C[C@@H](Oc1cc(-c2cc3n(n2)CCC32CCN(C(=O)OC(C)(C)C)C2)cnc1N)c1ccccc1]

Intermediate 251 was prepared in analogy to Intermediate 235 using 3-[(1R)-1-phenylethoxy]-5-(4,4,5,5-tetramethyl-1,3,2-dioxaborolan-2-yl)pyridin-2-amine (270 mg, 75% purity, 595 μmol) and (rac)-tert-butyl-2′-[(trifluoromethanesulfonyl)oxy]-5′,6′-dihydrospiro[pyrrolidine-3,4′-pyrrolo[1,2-b]pyrazole]-1-carboxylate (269 mg, 655 μmol).

LC-MS (method 1): Rt=1.26 min; MS (ESIpos): m/z=476 [M+H]⁺

¹H-NMR (400 MHz, DMSO-d6) δ[ppm]: 1.066 (16.00), 1.155 (0.95), 1.173 (2.06), 1.190 (1.04), 1.390 (2.91), 1.398 (3.03), 1.433 (3.31), 1.563 (2.54), 1.579 (2.49), 1.988 (3.70), 2.518 (1.08), 2.523 (0.83), 3.380 (1.36), 3.938 (2.63), 4.017 (0.73), 4.036 (0.70), 4.121 (0.59), 4.127 (0.59), 5.759 (5.03), 5.831 (1.48), 6.223 (0.41), 6.237 (0.42), 7.231 (0.92), 7.240 (0.58), 7.245 (0.51), 7.259 (0.42), 7.314 (0.58), 7.318 (0.60), 7.333 (1.06), 7.338 (0.98), 7.351 (0.53), 7.357 (0.47), 7.452 (1.30), 7.471 (0.92), 7.838 (1.50), 7.842 (1.43).

### Intermediate 252

### tert-butyl-2′-(6-amino-5-{[1-(5-chloro-3-fluoropyridin-2-yl)ethyl]oxy}pyridin-3-yl)-5′,6′-dihydrospiro[pyrrolidine-3,4′-pyrrolo[1,2-b]pyrazole]-1-carboxylate (Mixture of Isomers)

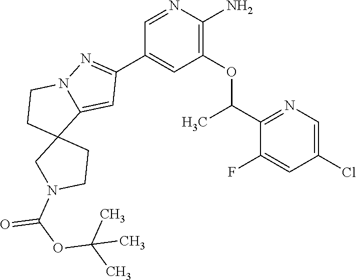 [SMILES: CC(Oc1cc(-c2cc3n(n2)CCC32CCN(C(=O)OC(C)(C)C)C2)cnc1N)c1ncc(Cl)cc1F]

Intermediate 252 was prepared in analogy to Intermediate 235 using (rac)-tert-butyl-2′-[(trifluoromethanesulfonyl)oxy]-5′,6′-dihydrospiro[pyrrolidine-3,4′-pyrrolo[1,2-b]pyrazole]-1-carboxylate (310 mg, 753 μmol) and (rac)-3-{[−1-(5-chloro-3-fluoropyridin-2-yl)ethyl]oxy}-5-(4,4,5,5-tetramethyl-1,3,2-dioxaborolan-2-yl)pyridin-2-amine (620 mg, 55% purity, 866 μmol).

LC-MS (method 2): Rt=1.26 min; MS (ESIpos): m/z=529 [M+H]⁺

### Intermediate 253

### (rac)-tert-butyl-2′-{6-amino-5-[(2-fluorophenyl)methoxy]pyridin-3-yl}-5′,6′-dihydrospiro[pyrrolidine-3,4′-pyrrolo[1,2-b]pyrazole]-1-carboxylate

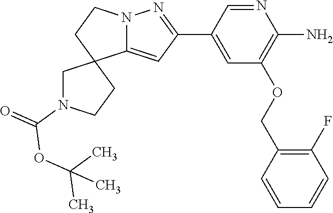 [SMILES: CC(C)(C)OC(=O)N1CCC2(CCn3nc(-c4cnc(N)c(OCc5ccccc5F)c4)cc32)C1]

(rac)-tert-butyl-2′-[(trifluoromethanesulfonyl)oxy]-5′,6′-dihydrospiro[pyrrolidine-3,4′-pyrrolo[1,2-b]pyrazole]-1-carboxylate (86.7 mg, 211 μmol), 3-[(2-fluorophenyl)methoxy]-5-(4,4,5,5-tetramethyl-1,3,2-dioxaborolan-2-yl)pyridin-2-amine (139 mg, 60% purity, 242 μmol), K3PO4 (1.3 mL, 0.50 M, 630 μmol), dicyclohexyl[2′,4′,6′-tri(propan-2-yl)[1,1′-biphenyl]-2-yl]phosphane (10.0 mg, 21.1 μmol) and XPhos Pd G2 (8.29 mg, 10.5 μmol) were stirred in degassed 1,4-dioxane (3.5 mL) for 1 h at 100° C. The mixture was diluted with water and extracted 3× with DCM. The combined organic layers were dried and evaporated. The residue was purified by flash chromatography to give 36.0 mg (36% yield) of the title compound.

LC-MS (method 1): Rt=1.23 min; MS (ESIpos): m/z=480 [M+H]⁺

### Intermediate 254

### (rac)-tert-butyl-2′-[6-amino-5-(benzyloxy)pyridin-3-yl]-5′,6′-dihydrospiro[pyrrolidine-3,4′-pyrrolo[1,2-b]pyrazole]-1-carboxylate

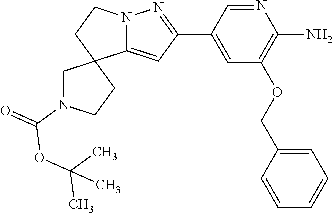 [SMILES: CC(C)(C)OC(=O)N1CCC2(CCn3nc(-c4cnc(N)c(OCc5ccccc5)c4)cc32)C1]

(rac)-tert-butyl-2′-[(trifluoromethanesulfonyl)oxy]-5′,6′-dihydrospiro[pyrrolidine-3,4′-pyrrolo[1,2-b]pyrazole]-1-carboxylate (80.1 mg, 195 μmol), 3-(benzyloxy)-5-(4,4,5,5-tetramethyl-1,3,2-dioxaborolan-2-yl)pyridin-2-amine (116 mg, 63% purity, 224 μmol), K3PO4 (1.2 mL, 0.50 M, 580 μmol), dicyclohexyl[2′,4′,6′-tri(propan-2-yl)[1,1′-biphenyl]-2-yl]phosphane (9.29 mg, 19.5 μmol) and XPhos Pd G2 (7.66 mg, 9.74 μmol) were stirred in degassed 1,4-dioxane (3.2 mL) for 1 h at 100° C. The mixture was diluted with water and extracted 3× with DCM. The combined organic layers were dried and evaporated. The residue was purified by flash chromatography to give 78.0 mg (87% yield) of the title compound.

LC-MS (method 1): Rt=1.23 min; MS (ESIpos): m/z=462 [M+H]⁺

1H-NMR (400 MHz, DMSO-d6): δ [ppm]=1.42 (d, 9H), 2.00-2.10 (m, 2H), 2.51-2.59 (m, 2H), 3.36-3.46 (m, 3H), 3.48-3.57 (m, 1H), 4.16 (td, 2H), 5.18 (s, 2H), 5.79 (s, 2H), 6.35 (d, 1H), 7.28-7.35 (m, 1H), 7.36-7.45 (m, 3H), 7.48-7.55 (m, 2H), 7.93 (d, 1H).

### Intermediate 255

### tert-butyl (3S)-2′-[6-amino-5-(pyridazin-3-ylmethoxy)pyridin-3-yl]-5′,6′-dihydro-1H-spiro[pyrrolidine-3,4′-pyrrolo[1,2-b]pyrazole]-1-carboxylate

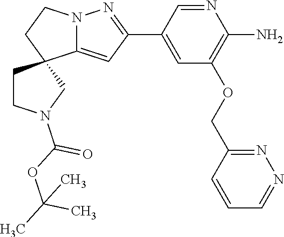 [SMILES: CC(C)(C)OC(=O)N1CC[C@]2(CCn3nc(-c4cnc(N)c(OCc5cccnn5)c4)cc32)C1]

Intermediate 255 was prepared in analogy to Intermediate 235 using tert-butyl (3S)-2′-[(trifluoromethanesulfonyl)oxy]-5′,6′-dihydrospiro[pyrrolidine-3,4′-pyrrolo[1,2-b]pyrazole]-1-carboxylate (100 mg, 244 μmol) and 3-[(pyridazin-3-yl)methoxy]-5-(4,4,5,5-tetramethyl-1,3,2-dioxaborolan-2-yl)pyridin-2-amine (206 mg, 51% purity, 317 μmol).

[α]²⁰_{D}: −39.2° (c=1.00, DMSO)

LC-MS (method 1): Rt=0.96 min; MS (ESIpos): m/z=464 [M+H]⁺

¹H NMR (400 MHz, DMSO-d6, 22° C.): δ=9.22 (dd, 1H), 8.01 (dd, 1H), 7.97 (d, 1H), 7.74-7.80 (m, 1H), 7.47-7.55 (m, 1H), 6.38 (d, 1H), 5.95 (s, 2H), 5.76 (s, 1H), 5.47 (s, 2H), 4.17 (td, 2H), 3.37-3.55 (m, 4H), 2.52-2.57 (m, 2H), 1.98-2.11 (m, 2H), 1.37-1.46 (m, 9H), 1.03-1.09 (m, 1H)

### Intermediate 256

### tert-butyl (3R)-2′-[6-amino-5-(pyridazin-3-ylmethoxy)pyridin-3-yl]-5′,6′-dihydro-1H-spiro[pyrrolidine-3,4′-pyrrolo[1,2-b]pyrazole]-1-carboxylate

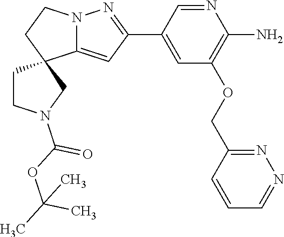 [SMILES: CC(C)(C)OC(=O)N1CC[C@@]2(CCn3nc(-c4cnc(N)c(OCc5cccnn5)c4)cc32)C1]

Intermediate 256 was prepared in analogy to Intermediate 235 using tert-butyl-(3R)-2′-[(trifluoromethanesulfonyl)oxy]-5′,6′-dihydrospiro[pyrrolidine-3,4′-pyrrolo[1,2-b]pyrazole]-1-carboxylate (100 mg, 244 μmol) and 3-[(pyridazin-3-yl)methoxy]-5-(4,4,5,5-tetramethyl-1,3,2-dioxaborolan-2-yl)pyridin-2-amine (206 mg, 51% purity, 317 μmol).

[α]²⁰_{D}: +37.1° (c=1.00, DMSO)

LC-MS (method 1): Rt=0.95 min; MS (ESIpos): m/z=464 [M+H]⁺

¹H NMR (400 MHz, DMSO-d6, 22° C.): δ=9.21 (d, J=4.8 Hz, 1H), 8.01 (dd, J=8.5, 1.6 Hz, 1H), 7.97 (d, J=1.8 Hz, 1H), 7.73-7.81 (m, 1H), 7.48-7.55 (m, 1H), 6.38 (d, J=9.9 Hz, 1H), 5.96 (s, 2H), 5.76 (s, 1H), 5.47 (s, 2H), 4.11-4.22 (m, 2H), 3.46-3.57 (m, 1H), 3.37-3.46 (m, 3H), 2.52-2.56 (m, 2H), 1.99-2.11 (m, 2H), 1.37-1.46 ppm (m, 8H)

### Intermediate 257

### tert-butyl (3S)-2′-{6-amino-5-[1-(pyrimidin-5-yl)ethoxy]pyridin-3-yl}-5′,6′-dihydro-1H-spiro[pyrrolidine-3,4′-pyrrolo[1,2-b]pyrazole]-1-carboxylate (Isomer 1)

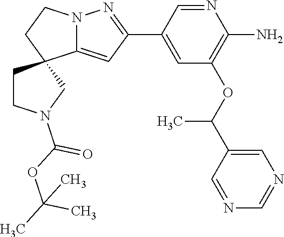 [SMILES: CC(Oc1cc(-c2cc3n(n2)CC[C@]32CCN(C(=O)OC(C)(C)C)C2)cnc1N)c1cncnc1]

Intermediate 257 was prepared in analogy to Intermediate 235 using tert-butyl-(3S)-2′-[(trifluoromethanesulfonyl)oxy]-5′,6′-dihydrospiro[pyrrolidine-3,4′-pyrrolo[1,2-b]pyrazole]-1-carboxylate (103 mg, 251 μmol, stereoisomer 1) and 3-{[1-(pyrimidin-5-yl)ethyl]oxy}-5-(4,4,5,5-tetramethyl-1,3,2-dioxaborolan-2-yl)pyridin-2-amine (186 mg, 60% purity, 326 μmol, stereoisomer 1).

[α]²⁰_{D}: −118.6° (c=1.00, DMSO)

LC-MS (method 1): Rt=0.99 min; MS (ESIpos): m/z=478 [M+H]⁺

¹H NMR (400 MHz, DMSO-d6, 22° C.): δ=9.11 (s, 1H), 8.98 (s, 2H), 7.91 (d, J=1.8 Hz, 1H), 7.41 (s, 1H), 6.32 (d, J=9.4 Hz, 1H), 5.96 (s, 2H), 5.78 (q, J=6.4 Hz, 1H), 4.09-4.21 (m, 2H), 3.35-3.54 (m, 4H), 1.98-2.11 (m, 2H), 1.62 (d, J=6.6 Hz, 3H), 1.36-1.46 ppm (m, 9H)

### Intermediate 258

### tert-butyl (3S)-2′-{6-amino-5-[1-(pyrimidin-5-yl)ethoxy]pyridin-3-yl}-5′,6′-dihydro-1H-spiro[pyrrolidine-3,4′-pyrrolo[1,2-b]pyrazole]-1-carboxylate (Isomer 1)

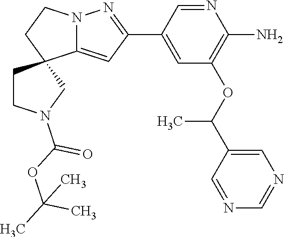 [SMILES: CC(Oc1cc(-c2cc3n(n2)CC[C@]32CCN(C(=O)OC(C)(C)C)C2)cnc1N)c1cncnc1]

Intermediate 258 was prepared in analogy to Intermediate 235 using tert-butyl (3S)-2′-[(trifluoromethanesulfonyl)oxy]-5′,6′-dihydrospiro[pyrrolidine-3,4′-pyrrolo[1,2-b]pyrazole]-1-carboxylate (96.5 mg, 235 μmol, stereoisomer 1) and 3-{[1-(pyrimidin-5-yl)ethyl]oxy}-5-(4,4,5,5-tetramethyl-1,3,2-dioxaborolan-2-yl)pyridin-2-amine (174 mg, 60% purity, 305 μmol, stereoisomer 2).

[α]²⁰_{D}: +37.6° (c=1.00, DMSO)

LC-MS (method 1): Rt=1.00 min; MS (ESIpos): m/z=478 [M+H]⁺

¹H NMR (400 MHz, DMSO-d6, 22° C.): δ=9.11 (s, 1H), 8.98 (s, 2H), 7.90 (s, 1H), 7.41 (s, 1H), 6.31 (d, J=9.6 Hz, 1H), 5.96 (s, 2H), 5.79 (q, J=6.2 Hz, 1H), 4.15 (td, J=6.9, 2.2 Hz, 2H), 3.45-3.56 (m, 1H), 3.36-3.44 (m, 3H), 2.52-2.56 (m, 2H), 1.98-2.10 (m, 2H), 1.62 (d, J=6.3 Hz, 3H), 1.37-1.46 ppm (m, 9H)

### Intermediate 259

### tert-butyl 2′-[6-amino-5-(trifluoromethyl)pyridin-3-yl]-5′,6′-dihydrospiro[azetidine-3,4′-pyrrolo[1,2-b]pyrazole]-1-carboxylate

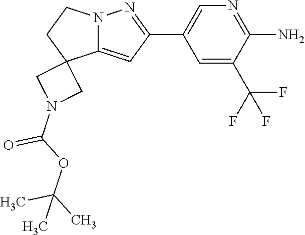 [SMILES: CC(C)(C)OC(=O)N1CC2(CCn3nc(-c4cnc(N)c(C(F)(F)F)c4)cc32)C1]

tert-butyl 2′-[(trifluoromethanesulfonyl)oxy]-5′,6′-dihydrospiro[azetidine-3,4′-pyrrolo[1,2-b]pyrazole]-1-carboxylate (2.00 g, 5.03 mmol) was solubilised in 1,4-dioxane (42 mL) under nitrogen, 5-(4,4,5,5-tetramethyl-1,3,2-dioxaborolan-2-yl)-3-(trifluoromethyl)pyridin-2-amine (2.17 g, 7.55 mmol), K3PO4 (30 mL, 0.50 M, 15 mmol) and XPhos Pd G2 (198 mg, 252 pmol) were added and the mixture was stirred overnight at 100° C. It was diluted with ethyl acetate, washed with brine, dried with a silicone filter and evaporated. The residue was purified by flash chromatography to give 2.10 g of the title compound.

LC-MS (method 1): Rt=1.19 min; MS (ESIpos): m/z=410 [M+H]⁺

1H-NMR (400 MHz, DMSO-d6): δ [ppm]=1.41 (s, 9H), 2.85 (t, 2H), 3.99-4.15 (m, 6H), 6.55 (s, 2H), 6.76 (s, 1H), 8.03 (d, 1H), 8.61 (d, 1H).

### Intermediate 260

### tert-butyl 2′-(6-amino-5-cyanopyridin-3-yl)-5′,6′-dihydrospiro[azetidine-3,4′-pyrrolo[1,2-b]pyrazole]-1-carboxylate

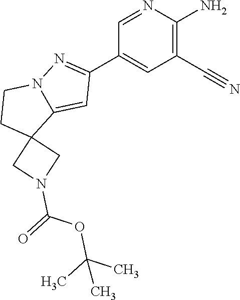 [SMILES: CC(C)(C)OC(=O)N1CC2(CCn3nc(-c4cnc(N)c(C#N)c4)cc32)C1]

Intermediate 260 was prepared in analogy to Intermediate 259 using tert-butyl 2′-[(trifluoromethanesulfonyl)oxy]-5′,6′-dihydrospiro[azetidine-3,4′-pyrrolo[1,2-b]pyrazole]-1-carboxylate (2.05 g, 5.17 mmol) and 2-amino-5-(4,4,5,5-tetramethyl-1,3,2-dioxaborolan-2-yl)pyridine-3-carbonitrile (1.90 g, 7.75 mmol).

LC-MS (method 1): Rₜ=1.06 min; MS (ESIpos): m/z=367 [M+H]⁺

¹H-NMR (400 MHz, DMSO-d6) δ[ppm]: 1.052 (0.57), 1.065 (2.31), 1.156 (0.63), 1.172 (0.51), 1.410 (16.00), 1.988 (0.83), 2.518 (2.02), 2.523 (1.39), 2.828 (0.58), 2.846 (0.95), 2.863 (0.64), 3.566 (2.05), 3.940 (0.40), 4.017 (0.56), 4.024 (0.61), 4.035 (0.42), 4.072 (0.63), 4.092 (1.03), 4.110 (1.07), 4.127 (0.64), 5.760 (4.70), 6.711 (2.96), 7.002 (1.50), 8.148 (1.31), 8.154 (1.43), 8.628 (1.42), 8.634 (1.51).

### Intermediate 261

### tert-butyl 2′-[6-amino-5-(difluoromethoxy)pyridin-3-yl]-5′,6′-dihydrospiro[azetidine-3,4′-pyrrolo[1,2-b]pyrazole]-1-carboxylate

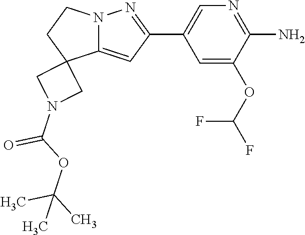 [SMILES: CC(C)(C)OC(=O)N1CC2(CCn3nc(-c4cnc(N)c(OC(F)F)c4)cc32)C1]

Intermediate 261 was prepared in analogy to Intermediate 259 using tert-butyl 2′-[(trifluoromethanesulfonyl)oxy]-5′,6′-dihydrospiro[azetidine-3,4′-pyrrolo[1,2-b]pyrazole]-1-carboxylate (2.41 g, 6.06 mmol) and 3-(difluoromethoxy)-5-(4,4,5,5-tetramethyl-1,3,2-dioxaborolan-2-yl)pyridin-2-amine (2.60 g, 9.09 mmol).

LC-MS (method 1): Rt=1.11 min; MS (ESIpos): m/z=408 [M+H]⁺

1H-NMR (400 MHz, DMSO-d6) δ[ppm]: 1.066 (16.00), 1.155 (0.75), 1.173 (1.59), 1.190 (0.80), 1.409 (4.36), 1.988 (2.82), 2.518 (0.39), 2.523 (0.26), 2.562 (0.27), 2.843 (0.25), 2.860 (0.16), 2.888 (0.17), 3.937 (2.60), 4.000 (0.22), 4.017 (0.69), 4.035 (0.72), 4.053 (0.29), 4.065 (0.19), 4.085 (0.26), 4.103 (0.28), 4.120 (0.17), 6.165 (0.40), 6.656 (0.79), 6.996 (0.17), 7.181 (0.35), 7.634 (0.22), 7.636 (0.22), 8.228 (0.40), 8.232 (0.36).

### Intermediate 262

### tert-butyl 2′-[6-amino-5-(trifluoromethoxy)pyridin-3-yl]-5′,6′-dihydrospiro[azetidine-3,4′-pyrrolo[1,2-b]pyrazole]-1-carboxylate

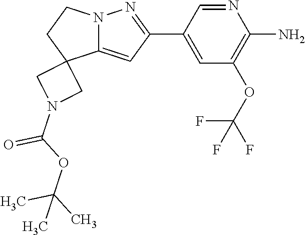 [SMILES: CC(C)(C)OC(=O)N1CC2(CCn3nc(-c4cnc(N)c(OC(F)(F)F)c4)cc32)C1]

Intermediate 262 was prepared in analogy to Intermediate 259 using tert-butyl 2′-[(trifluoromethanesulfonyl)oxy]-5′,6′-dihydrospiro[azetidine-3,4′-pyrrolo[1,2-b]pyrazole]-1-carboxylate (816 mg, 2.05 mmol) and [6-amino-5-(trifluoromethoxy)pyridin-3-yl]boronic acid (1.14 g, 60% purity, 3.08 mmol).

LC-MS (method 1): Rₜ=1.20 min; MS (ESIpos): m/z=426 [M+H]⁺

¹H-NMR (400 MHz, DMSO-d6) δ[ppm]: 1.066 (16.00), 1.173 (0.54), 1.409 (2.71), 1.988 (1.03), 3.938 (2.70), 6.701 (0.52).

### Intermediate 263

### tert-butyl 2′-{6-amino-5-[(1S)-1-(pyridin-2-yl)ethoxy]pyridin-3-yl}-5′,6′-dihydrospiro[azetidine-3,4′-pyrrolo[1,2-b]pyrazole]-1-carboxylate

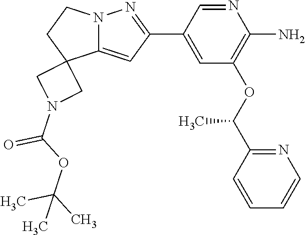 [SMILES: C[C@H](Oc1cc(-c2cc3n(n2)CCC32CN(C(=O)OC(C)(C)C)C2)cnc1N)c1ccccn1]

Intermediate 263 was prepared in analogy to Intermediate 259 using tert-butyl 2′-[(trifluoromethanesulfonyl)oxy]-5′,6′-dihydrospiro[azetidine-3,4′-pyrrolo[1,2-b]pyrazole]-1-carboxylate (122 mg, 307 μmol) and 3-[(1S)-1-(pyridin-2-yl)ethoxy]-5-(4,4,5,5-tetramethyl-1,3,2-dioxaborolan-2-yl)pyridin-2-amine (105 mg, 307 μmol).

¹H-NMR (400 MHz, DMSO-d6): δ [ppm]=1.41 (s, 9H), 1.62 (br d, 3H), 2.81 (br t, 2H), 3.97-4.15 (m, 6H), 5.53 (q, 1H), 5.90 (s, 2H), 6.52 (s, 1H), 7.24 (s, 1H), 7.26-7.33 (m, 1H), 7.51 (br d, 1H), 7.79 (br t, 1H), 7.90 (s, 1H), 8.55 (br d, 1H).

LC-MS (method 1): Rₜ=1.10 min; MS (ESIpos): m/z=463 [M+H]⁺

### Intermediate 264

### tert-butyl 2′-{6-amino-5-[(1R)-1-(pyridin-2-yl)ethoxy]pyridin-3-yl}-5′,6′-dihydrospiro[azetidine-3,4′-pyrrolo[1,2-b]pyrazole]-1-carboxylate

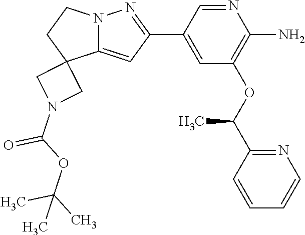 [SMILES: C[C@@H](Oc1cc(-c2cc3n(n2)CCC32CN(C(=O)OC(C)(C)C)C2)cnc1N)c1ccccn1]

Intermediate 264 was prepared in analogy to Intermediate 259 using tert-butyl 2′-[(trifluoromethanesulfonyl)oxy]-5′,6′-dihydrospiro[azetidine-3,4′-pyrrolo[1,2-b]pyrazole]-1-carboxylate (175 mg, 441 μmol) and 3-[(1R)-1-(pyridin-2-yl)ethoxy]-5-(4,4,5,5-tetramethyl-1,3,2-dioxaborolan-2-yl)pyridin-2-amine (150 mg, 441 μmol).

LC-MS (method 1): Rₜ=1.11 min; MS (ESIpos): m/z=463 [M+H]⁺

### Intermediate 265

### tert-butyl 2′-{6-amino-5-[(1R)-1-phenylethoxy]pyridin-3-yl}-5′,6′-dihydrospiro[azetidine-3,4′-pyrrolo[1,2-b]pyrazole]-1-carboxylate

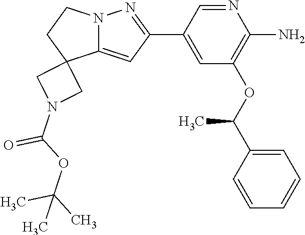 [SMILES: C[C@@H](Oc1cc(-c2cc3n(n2)CCC32CN(C(=O)OC(C)(C)C)C2)cnc1N)c1ccccc1]

Intermediate 265 was prepared in analogy to Intermediate 259 using tert-butyl 2′-[(trifluoromethanesulfonyl)oxy]-5′,6′-dihydrospiro[azetidine-3,4′-pyrrolo[1,2-b]pyrazole]-1-carboxylate (175 mg, 441 μmol) and 3-[(1R)-1-phenylethoxy]-5-(4,4,5,5-tetramethyl-1,3,2-dioxaborolan-2-yl)pyridin-2-amine (150 mg, 441 μmol).

LC-MS (method 1): Rₜ=1.25 min; MS (ESIpos): m/z=462 [M+H]⁺

### Intermediate 266

### tert-butyl 2′-{6-amino-5-[(1S)-1-phenylethoxy]pyridin-3-yl}-5′,6′-dihydrospiro[azetidine-3,4′-pyrrolo[1,2-b]pyrazole]-1-carboxylate

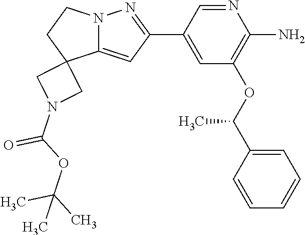 [SMILES: C[C@H](Oc1cc(-c2cc3n(n2)CCC32CN(C(=O)OC(C)(C)C)C2)cnc1N)c1ccccc1]

Intermediate 266 was prepared in analogy to Intermediate 259 using tert-butyl 2′-[(trifluoromethanesulfonyl)oxy]-5′,6′-dihydrospiro[azetidine-3,4′-pyrrolo[1,2-b]pyrazole]-1-carboxylate (175 mg, 441 μmol) and 3-[(1S)-1-phenylethoxy]-5-(4,4,5,5-tetramethyl-1,3,2-dioxaborolan-2-yl)pyridin-2-amine (150 mg, 441 μmol).

LC-MS (method 1): Rₜ=1.27 min; MS (ESIpos): m/z=462 [M+H]⁺

### Intermediate 267

### (rac)-tert-butyl-2-(6-amino-5-cyanopyridin-3-yl)-6,7-dihydrospiro[pyrazolo[5,1-c][1,4]oxazine-4,3′-pyrrolidine]-1′-carboxylate

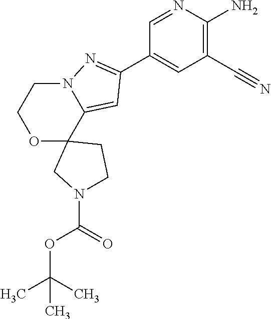 [SMILES: CC(C)(C)OC(=O)N1CCC2(C1)OCCn1nc(-c3cnc(N)c(C#N)c3)cc12]

(Rac)-tert-butyl-2-bromo-6,7-dihydrospiro[pyrazolo[5,1-c][1,4]oxazine-4,3′-pyrrolidine]-1′-carboxylate (621 mg, 1.73 mmol), 2-amino-5-(4,4,5,5-tetramethyl-1,3,2-dioxaborolan-2-yl)pyridine-3-carbonitrile (850 mg, 3.47 mmol), XPhos Pd G2 (68.2 mg, 86.7 μmol) and potassium phosphate (10 mL, 0.50 M, 5.2 mmol) were stirred in degassed dioxane (25 mL) for 2 h at 100° C. The reaction mixture was diluted with ethyl acetate and water. The phases were separated and the aqueous phase was extracted twice with ethyl acetate. The combined organic phases were dried over sodium sulfate and concentrated. The residue was purified by flash chromatography to afford 736 mg of the title compound.

¹H-NMR (400 MHz, DMSO-d6) δ[ppm]: 1.065 (4.11), 1.265 (0.50), 1.399 (7.96), 1.424 (6.94), 2.518 (0.73), 2.523 (0.49), 3.159 (16.00), 3.172 (15.27), 3.366 (0.44), 3.375 (0.41), 3.397 (0.49), 3.428 (0.42), 3.483 (0.44), 3.551 (0.65), 3.726 (0.52), 3.941 (0.69), 4.087 (1.54), 4.100 (4.02), 4.113 (6.91), 4.126 (1.93), 6.699 (0.95), 6.717 (1.09), 7.046 (2.34), 8.114 (1.17), 8.119 (1.15), 8.591 (2.11), 8.598 (2.12).

### Intermediate 268

### (rac)-tert-butyl-2-[6-amino-5-(trifluoromethoxy)pyridin-3-yl]-6,7-dihydrospiro[pyrazolo[5,1-c][1,4]oxazine-4,3′-pyrrolidine]-1′-carboxylate

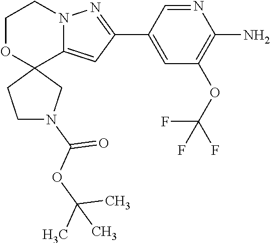 [SMILES: CC(C)(C)OC(=O)N1CCC2(C1)OCCn1nc(-c3cnc(N)c(OC(F)(F)F)c3)cc12]

Intermediate 268 was prepared in analogy to Intermediate 235 using (rac)-tert-butyl-2-bromo-6,7-dihydrospiro[pyrazolo[5,1-c][1,4]oxazine-4,3′-pyrrolidine]-1′-carboxylate (1.13 g, 3.15 mmol) and [6-amino-5-(trifluoromethoxy)pyridin-3-yl]boronic acid (1.75 g, 60% purity, 4.73 mmol).

LC-MS (method 1): Rt=1.18 min; MS (ESIpos): m/z=456 [M+H]⁺

### Intermediate 269

### (rac)-tert-butyl-2-[6-amino-5-(difluoromethoxy)pyridin-3-yl]-6,7-dihydrospiro[pyrazolo[5,1-c][1,4]oxazine-4,3′-pyrrolidine]-1′-carboxylate

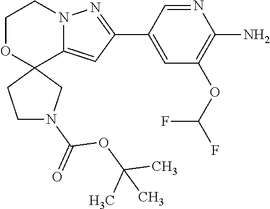 [SMILES: CC(C)(C)OC(=O)N1CCC2(C1)OCCn1nc(-c3cnc(N)c(OC(F)F)c3)cc12]

(rac)-tert-butyl-2-bromo-6,7-dihydrospiro[pyrazolo[5,1-c][1,4]oxazine-4,3′-pyrrolidine]-1′-carboxylate (1.05 g, 2.94 mmol) was solubilized in 1,4-dioxane (29 mL), [6-amino-5-(difluoromethoxy)pyridin-3-yl]boronic acid (1.50 g, 60% purity, 4.41 mmol), K3PO4 (18 mL, 0.50 M, 8.8 mmol) and XPhos Pd G2 (347 mg, 441 μmol) were added and the mixture was stirred overnight at 100° C. It was diluted with water and extracted 2× with ethyl acetate. The combined organic layers were dried over a silicone filter and evaporated. The residue was purified by flash chromatography to give 1.39 g of the title compound.

LC-MS (method 1): Rt=1.11 min; MS (ESIpos): m/z=438 [M+H]⁺

¹H-NMR (400 MHz, DMSO-d6) δ[ppm]: 1.066 (16.00), 1.401 (3.24), 1.425 (2.79), 2.518 (1.46), 2.523 (1.02), 3.939 (2.63), 4.110 (1.50), 6.218 (0.87), 6.676 (0.43), 7.179 (0.72), 7.605 (0.48), 8.188 (0.56), 8.192 (0.54).

### Intermediate 270

### tert-butyl (3′R)-2-[6-amino-5-(trifluoromethoxy)pyridin-3-yl]-6,7-dihydrospiro[pyrazolo[5,1-c][1,4]oxazine-4,3′-pyrrolidine]-1′-carboxylate

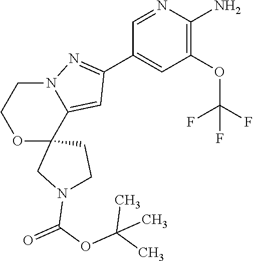 [SMILES: CC(C)(C)OC(=O)N1CC[C@]2(C1)OCCn1nc(-c3cnc(N)c(OC(F)(F)F)c3)cc12]

To a solution of 5-(4,4,5,5-tetramethyl-1,3,2-dioxaborolan-2-yl)-3-(trifluoromethoxy)pyridin-2-amine (357 mg, 1.17 mmol) in Dioxane (12 mL) under Argon, tert-butyl (3′R)-2-bromo-6,7-dihydrospiro[pyrazolo[5,1-c][1,4]oxazine-4,3′-pyrrolidine]-1′-carboxylate (442 mg, 1.23 mmol) and Potassium phosphate (7.0 ml, 0.50 M in water, 3.5 mmol) were added. The mixture was degassed and XPhos Pd G2 (46.2 mg, 58.7 μmol) was added and stirred at 100° C. under Argon for 1 h. The mixture was diluted with water and then extracted with Ethyl acetate 2×. The combined organics were washed with Brine, dried over Sodium sulfate, filtered and concentrated to give 602 mg of the title compound.

LC-MS (Method 1): Rₜ=1.20 min; MS (ESIpos): m/z=456 [M+H]⁺

¹H-NMR (400 MHz, DMSO-d6) δ[ppm]: 1.066 (16.00), 1.156 (1.47), 1.400 (1.23), 1.424 (1.01), 3.565 (15.63), 3.938 (2.49), 4.112 (0.41).

### Intermediate 271

### tert-butyl (3R)-2′-[6-amino-5-(trifluoromethyl)pyridin-3-yl]-5′,6′-dihydrospiro[pyrrolidine-3,4′-pyrrolo[1,2-b]pyrazole]-1-carboxylate

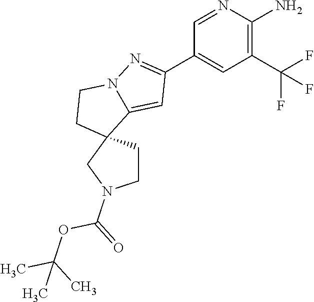 [SMILES: CC(C)(C)OC(=O)N1CC[C@@]2(CCn3nc(-c4cnc(N)c(C(F)(F)F)c4)cc32)C1]

To a crude solution of 5-(4,4,5,5-tetramethyl-1,3,2-dioxaborolan-2-yl)-3-(trifluoromethyl)pyridin-2-amine (2.00 g, 6.94 mmol), tert-butyl (3R)-2′-[(trifluoromethanesulfonyl)oxy]-5′,6′-dihydrospiro[pyrrolidine-3,4′-pyrrolo[1,2-b]pyrazole]-1-carboxylate (3.00 g, 7.29 mmol) and Potassium phosphate (42 ml, 0.50 M in water, 21 mmol) were added. The mixture was degassed and XPhos Pd G2 (273 mg, 347 μmol) was added and stirred at 100° C. under Argon for 2 h. The mixture was diluted with water and then extracted with Ethyl acetate 2×. The combined organics were washed with Brine, dried over Sodium sulfate, filtered and concentrated. The crude was purified by column chromatography to give 3.50 g of the title compound.

LC-MS (Method 1): Rₜ=1.20 min; MS (ESIpos): m/z=425 [M+H]⁺

### Intermediate 272

### tert-butyl (3′R)-2-[6-amino-5-(difluoromethoxy)pyridin-3-yl]-6,7-dihydrospiro[pyrazolo[5,1-c][1,4]oxazine-4,3′-pyrrolidine]-1′-carboxylate

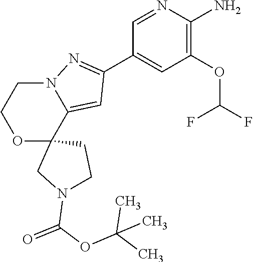 [SMILES: CC(C)(C)OC(=O)N1CC[C@]2(C1)OCCn1nc(-c3cnc(N)c(OC(F)F)c3)cc12]

To a solution of 3-(difluoromethoxy)-5-(4,4,5,5-tetramethyl-1,3,2-dioxaborolan-2-yl)pyridin-2-amine (250 mg, 874 μmol) in Dioxane (9 mL), tert-butyl (3′R)-2-bromo-6,7-dihydrospiro[pyrazolo[5,1-c][1,4]oxazine-4,3′-pyrrolidine]-1′-carboxylate (344 mg, 961 μmol) and Potassium phosphate (5.2 ml, 0.50 M in water, 2.6 mmol) were added. The mixture was degassed and XPhos Pd G2 (34.4 mg, 43.7 μmol) was added. The mixture was stirred at 100° C. under Argon for 1 h. The mixture was diluted with water and then extracted with Ethyl acetate 2×. The combined organics were washed with Brine, dried over Sodium sulfate, filtered and concentrated in vacuo to give 423 mg of the title compound.

H-NMR-Data-1H NMR (DMSO-d6, 400 MHz) δ 8.19 (d, 1H), 7.61 (s, 1H), 7.0-7.4 (m, 1H), 6.67 (d, 1H), 6.22 (s, 2H), 4.11 (s, 4H), 3.7-3.8 (m, 1H), 3.4-3.6 (m, 3H), 2.2-2.3 (m, 1H), 1.07 (s, 9H)

LC-MS (Method 1): Rₜ=1.11 min; MS (ESIpos): m/z=439 [M+H]⁺

¹H-NMR (400 MHz, DMSO-d6) δ[ppm]: 1.066 (16.00), 1.401 (2.51), 1.425 (2.11), 2.518 (0.45), 3.333 (13.40), 3.939 (2.32), 4.110 (1.11), 6.217 (0.68), 7.178 (0.64), 8.189 (0.44), 8.193 (0.43).

### Intermediate 273

### tert-butyl (3R)-2′-[6-amino-5-(difluoromethoxy)pyridin-3-yl]-5′,6′-dihydrospiro[pyrrolidine-3,4′-pyrrolo[1,2-b]pyrazole]-1-carboxylate

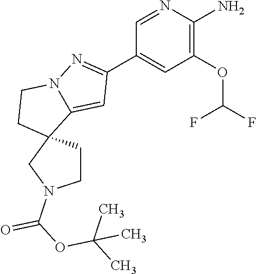 [SMILES: CC(C)(C)OC(=O)N1CC[C@@]2(CCn3nc(-c4cnc(N)c(OC(F)F)c4)cc32)C1]

To a solution of 3-(difluoromethoxy)-5-(4,4,5,5-tetramethyl-1,3,2-dioxaborolan-2-yl)pyridin-2-amine (250 mg, 874 μmol) in Dioxane (9 mL), tert-butyl (3R)-2′-[(trifluoromethanesulfonyl)oxy]-5′,6′-dihydrospiro[pyrrolidine-3,4′-pyrrolo[1,2-b]pyrazole]-1-carboxylate (395 mg, 961 μmol) and Potassium phosphate (5.2 ml, 0.50 M in water, 2.6 mmol) were added. The mixture was degassed and XPhos Pd G2 (34.4 mg, 43.7 μmol) was added and stirred at 100° C. under Argon for 1 h. The mixture was diluted with water and then extracted with Ethyl acetate 2×. The combined organics were washed with Brine, dried over Sodium sulfate, filtered and concentrated to give 401 mg of the title compound.

1H NMR (DMSO-d6, 400 MHz) δ 8.20 (d, 1H), 7.62 (s, 1H), 7.0-7.4 (m, 1H), 6.39 (d, 1H), 6.16 (s, 2H), 4.17 (dt, 2H), 3.51 (br d, 1H), 3.4-3.4 (m, 3H), 2.0-2.1 (m, 2H), 1.07 (s, 10H)

LC-MS (Method 1): Rₜ=1.12 min; MS (ESIpos): m/z=423 [M+H]⁺

¹H-NMR (400 MHz, DMSO-d6) δ[ppm]: 1.066 (16.00), 1.156 (0.45), 1.396 (2.13), 1.433 (1.66), 2.518 (0.44), 3.333 (6.64), 3.409 (0.77), 3.940 (1.29), 6.158 (0.60), 7.179 (0.60), 8.201 (0.58), 8.205 (0.61).

### Intermediate 274

### tert-butyl (3′R)-2-[6-amino-5-(trifluoromethyl)pyridin-3-yl]-6,7-dihydrospiro[pyrazolo[5,1-c][1,4]oxazine-4,3′-pyrrolidine]-1′-carboxylate

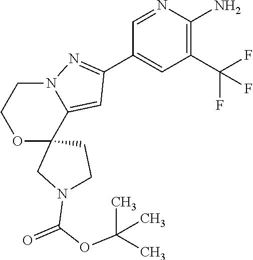 [SMILES: CC(C)(C)OC(=O)N1CC[C@]2(C1)OCCn1nc(-c3cnc(N)c(C(F)(F)F)c3)cc12]

To a solution of 5-(4,4,5,5-tetramethyl-1,3,2-dioxaborolan-2-yl)-3-(trifluoromethyl)pyridin-2-amine (500 mg, 1.74 mmol) in Dioxane (18 mL), tert-butyl (3′R)-2-bromo-6,7-dihydrospiro[pyrazolo[5,1-c][1,4]oxazine-4,3′-pyrrolidine]-1′-carboxylate (933 mg, 2.60 mmol) and Potassium phosphate (10 ml, 0.50 M in water, 5.2 mmol) were added. The mixture was degassed and XPhos Pd G2 (68.3 mg, 86.8 μmol) was added and stirred at 100° C. under Argon for 1 h. The mixture was diluted with water and then extracted with Ethyl acetate 2×. The combined organics were washed with Brine, dried over Sodium sulfate and concentrated in vacuo. The crude was purified by column chromatography to give 448 mg (59% yield) of the title compound.

1H NMR (DMSO-d6, 400 MHz) δ 8.57 (d, 1H), 8.01 (s, 1H), 6.75 (d, 1H), 6.60 (s, 2H), 4.1-4.2 (m, 4H), 3.7-3.8 (m, 1H), 3.5-3.6 (m, 1H), 3.4-3.5 (m, 1H), 3.4-3.4 (m, 1H), 2.1-2.3 (m, 1H), 1.4-1.4 (m, 9H)

LC-MS (Method 1): Rₜ=1.21 min; MS (ESIpos): m/z=440 [M+H]⁺

¹H-NMR (400 MHz, DMSO-d6) δ[ppm]: 1.066 (1.70), 1.401 (16.00), 1.415 (4.28), 1.425 (13.53), 2.318 (0.88), 2.322 (1.31), 2.326 (1.49), 2.332 (1.23), 2.336 (0.77), 2.518 (5.12), 2.523 (3.38), 2.664 (0.86), 2.669 (1.14), 2.673 (0.86), 3.159 (0.70), 3.171 (0.71), 3.345 (1.03), 3.364 (0.66), 3.376 (0.67), 3.392 (0.56), 3.421 (0.88), 3.451 (0.88), 3.476 (0.78), 3.505 (1.02), 3.527 (0.80), 3.550 (1.24), 3.573 (0.54), 3.695 (0.68), 3.727 (1.00), 3.757 (0.45), 4.084 (1.08), 4.095 (0.88), 4.119 (5.84), 6.598 (4.08), 6.744 (1.86), 6.765 (2.21), 8.005 (2.10), 8.572 (2.18), 8.576 (2.14).

### Intermediate 275

### tert-butyl (3'S)-2-[6-amino-5-(trifluoromethyl)pyridin-3-yl]-6,7-dihydro-1′H-spiro[pyrazolo[5,1-c][1,4]oxazine-4,3′-pyrrolidine]-1′-carboxylate

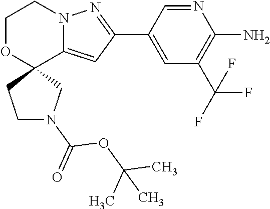 [SMILES: CC(C)(C)OC(=O)N1CC[C@@]2(C1)OCCn1nc(-c3cnc(N)c(C(F)(F)F)c3)cc12]

Intermediate 275 was prepared in analogy to Intermediate 270 using tert-butyl-(3'S)-2-bromo-6,7-dihydrospiro[pyrazolo[5,1-c][1,4]oxazine-4,3′-pyrrolidine]-1′-carboxylate (150 mg, 419 μmol, stereoisomer 1) and 5-(4,4,5,5-tetramethyl-1,3,2-dioxaborolan-2-yl)-3-(trifluoromethyl)pyridin-2-amine (181 mg, 628 μmol).

LC-MS (method 1): Rₜ=1.20 min; MS (ESIpos): m/z=440 [M+H]⁺

¹H-NMR (400 MHz, DMSO-d6) δ[ppm]: 1.066 (3.40), 1.156 (1.42), 1.232 (0.40), 1.401 (13.73), 1.425 (11.60), 2.085 (7.46), 2.323 (1.19), 2.327 (1.36), 2.331 (1.10), 2.518 (3.51), 2.523 (2.25), 2.665 (0.70), 2.669 (0.97), 2.673 (0.67), 3.365 (0.53), 3.375 (0.52), 3.391 (0.45), 3.421 (0.64), 3.451 (0.66), 3.476 (0.62), 3.506 (0.78), 3.528 (0.62), 3.551 (1.08), 3.573 (0.47), 3.697 (0.56), 3.726 (0.88), 3.939 (0.56), 4.119 (5.22), 5.759 (16.00), 6.600 (3.62), 6.746 (1.63), 6.766 (1.92), 8.006 (1.94), 8.572 (1.92), 8.577 (1.88).

### Intermediate 276

### tert-butyl (3'S)-2-(6-amino-5-cyanopyridin-3-yl)-6,7-dihydro-1′H-spiro[pyrazolo[5,1-c][1,4]oxazine-4,3′-pyrrolidine]-1′-carboxylate

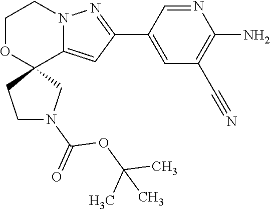 [SMILES: CC(C)(C)OC(=O)N1CC[C@@]2(C1)OCCn1nc(-c3cnc(N)c(C#N)c3)cc12]

Intermediate 276 was prepared in analogy to Intermediate 270 using tert-butyl-(3'S)-2-bromo-6,7-dihydrospiro[pyrazolo[5,1-c][1,4]oxazine-4,3′-pyrrolidine]-1′-carboxylate (250 mg, 698 μmol, stereoisomer 1) and 2-amino-5-(4,4,5,5-tetramethyl-1,3,2-dioxaborolan-2-yl)pyridine-3-carbonitrile (257 mg, 1.05 mmol).

LC-MS (method 1): Rt=1.08 min; MS (ESIpos): m/z=397 [M+H]⁺

1H-NMR (400 MHz, DMSO-d6) δ[ppm]: 1.066 (2.44), 1.154 (1.13), 1.172 (2.22), 1.190 (1.08), 1.401 (16.00), 1.425 (14.03), 1.988 (3.85), 2.178 (0.42), 2.205 (0.45), 2.322 (1.05), 2.327 (1.22), 2.332 (1.02), 2.518 (2.92), 2.523 (1.80), 2.665 (0.50), 2.669 (0.73), 2.673 (0.51), 3.366 (0.71), 3.377 (0.67), 3.397 (0.97), 3.428 (0.82), 3.454 (0.75), 3.483 (0.88), 3.529 (0.76), 3.551 (1.31), 3.575 (0.57), 3.696 (0.67), 3.727 (1.06), 3.758 (0.49), 3.938 (0.46), 4.017 (0.92), 4.035 (0.92), 4.115 (7.84), 6.699 (1.92), 6.718 (2.22), 7.045 (4.84), 8.114 (2.41), 8.119 (2.39), 8.591 (4.30), 8.597 (4.25).

### Intermediate 277

### tert-butyl (3′R)-2-(6-amino-5-cyanopyridin-3-yl)-6,7-dihydro-1′H-spiro[pyrazolo[5,1-c][1,4]oxazine-4,3′-pyrrolidine]-1′-carboxylate

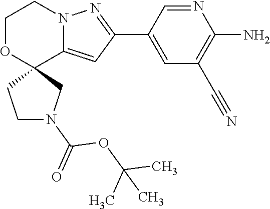 [SMILES: CC(C)(C)OC(=O)N1CC[C@]2(C1)OCCn1nc(-c3cnc(N)c(C#N)c3)cc12]

Intermediate 277 was prepared in analogy to Intermediate 270 using tert-butyl-(3′R)-2-bromo-6,7-dihydrospiro[pyrazolo[5,1-c][1,4]oxazine-4,3′-pyrrolidine]-1′-carboxylate (300 mg, 837 μmol, stereoisomer 2) and 2-amino-5-(4,4,5,5-tetramethyl-1,3,2-dioxaborolan-2-yl)pyridine-3-carbonitrile (308 mg, 1.26 mmol).

LC-MS (method 1): Rₜ=1.06 min; MS (ESIneg): m/z=395 [M−H]⁻

¹H-NMR (400 MHz, DMSO-d6) δ[ppm]: 1.066 (16.00), 1.154 (0.66), 1.172 (1.22), 1.190 (0.61), 1.401 (1.85), 1.425 (1.55), 1.988 (2.25), 2.518 (0.85), 2.523 (0.59), 3.938 (2.92), 4.017 (0.52), 4.035 (0.52), 4.115 (0.86), 5.758 (2.26), 7.045 (0.53), 8.591 (0.56), 8.597 (0.50).

### Intermediate 278

### (rac)-tert-butyl-2′-(6-amino-5-cyanopyridin-3-yl)-5′,6′-dihydrospiro[pyrrolidine-3,4′-pyrrolo[1,2-b]pyrazole]-1-carboxylate

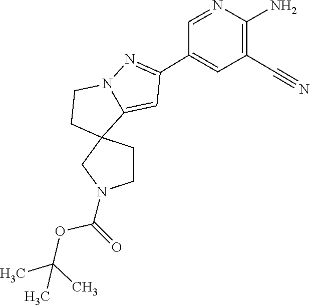 [SMILES: CC(C)(C)OC(=O)N1CCC2(CCn3nc(-c4cnc(N)c(C#N)c4)cc32)C1]

(Rac)-tert-butyl-2′-[(trifluoromethanesulfonyl)oxy]-5′,6′-dihydrospiro[pyrrolidine-3,4′-pyrrolo[1,2-b]pyrazole]-1-carboxylate (713 mg, 1.73 mmol), 2-amino-5-(4,4,5,5-tetramethyl-1,3,2-dioxaborolan-2-yl)pyridine-3-carbonitrile (850 mg, 3.47 mmol), XPhos Pd G2 (68.2 mg, 86.7 μmol) and potassium phosphate (10 mL, 0.50 M, 5.2 mmol) were dissolved in degassed dioxane (25 mL) under argon and stirred at 100° C. for 2 h. The mixture was diluted with ethyl acetate and water, the layers were separated and the aqueous phase was extracted twice with ethyl acetate. The combined organic phases were dried over sodium sulfate and concentrated. Purified by flash chromatography to afford 730 mg of the title compound.

¹H-NMR (400 MHz, DMSO-d6) δ[ppm]: 1.065 (2.09), 1.396 (7.48), 1.432 (6.06), 2.029 (0.79), 2.047 (0.64), 2.518 (0.66), 2.523 (0.65), 2.534 (0.65), 2.542 (0.75), 2.560 (0.48), 3.159 (16.00), 3.172 (15.80), 3.386 (0.65), 3.407 (2.03), 3.434 (0.62), 3.496 (0.64), 4.087 (1.45), 4.101 (3.63), 4.113 (3.70), 4.127 (1.34), 4.154 (0.51), 4.163 (0.62), 4.172 (0.92), 4.181 (0.90), 4.189 (0.62), 4.198 (0.49), 6.454 (0.82), 6.479 (1.01), 6.985 (2.41), 8.151 (1.16), 8.156 (1.10), 8.611 (1.97), 8.618 (1.97).

### Intermediate 279

### (rac)-5-[5′,6′-dihydrospiro[pyrrolidine-3,4′-pyrrolo[1,2-b]pyrazol]-2′-yl]-3-(trifluoromethoxy)pyridin-2-amine

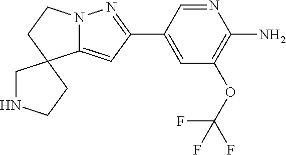 [SMILES: Nc1ncc(-c2cc3n(n2)CCC32CCNC2)cc1OC(F)(F)F]

(rac)-tert-butyl-2′-[6-amino-5-(trifluoromethoxy)pyridin-3-yl]-5′,6′-dihydrospiro[pyrrolidine-3,4′-pyrrolo[1,2-b]pyrazole]-1-carboxylate (590 mg, 1.34 mmol) was solubilised in 1,4-dioxane (12 mL), HCl (3.4 mL, 4.0 M in 1,4-dioxane, 13 mmol) was added and the mixture was stirred overnight at rt. HCl (1.7 mL, 4.0 M, 6.7 mmol) was added and the mixture was stirred for 1 h at 50° C. The mixture was concentrated under reduced pressure to give 590 mg of the title compound.

LC-MS (method 1): Rt=0.85 min; MS (ESIpos): m/z=340 [M+H]⁺

### Intermediate 280

### (rac)-3-(difluoromethoxy)-5-[5′,6′-dihydrospiro[pyrrolidine-3,4′-pyrrolo[1,2-b]pyrazol]-2′-yl]pyridin-2-amine-hydrochloride salt

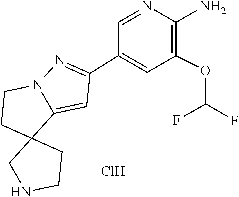 [SMILES: Nc1ncc(-c2cc3n(n2)CCC32CCNC2)cc1OC(F)F ; Cl]

Intermediate 280 was prepared in analogy to Intermediate 279 using (rac)-tert-butyl-2′-[6-amino-5-(difluoromethoxy)pyridin-3-yl]-5′,6′-dihydrospiro[pyrrolidine-3,4′-pyrrolo[1,2-b]pyrazole]-1-carboxylate (1.36 g, 3.23 mmol).

LC-MS (method 1): Rt=0.77 min; MS (ESIpos): m/z=323 [M+H]⁺

### Intermediate 281

### 5-[5′,6′-dihydrospiro[pyrrolidine-3,4′-pyrrolo[1,2-b]pyrazol]-2′-yl]-3-[(1R)-1-(pyridin-3-yl)ethoxy]pyridin-2-amine-hydrochloride salt

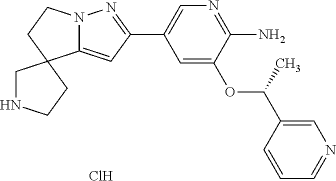 [SMILES: C[C@@H](Oc1cc(-c2cc3n(n2)CCC32CCNC2)cnc1N)c1cccnc1 ; Cl]

Intermediate 281 was prepared in analogy to Intermediate 279 using (rac)-tert-butyl-2′-{6-amino-5-[(1R)-1-(pyridin-3-yl)ethoxy]pyridin-3-yl}-5′,6′-dihydrospiro[pyrrolidine-3,4′-pyrrolo[1,2-b]pyrazole]-1-carboxylate (55.0 mg, 115 μmol).

LC-MS (method 1): Rt=0.77 min; MS (ESIpos): m/z=377 [M+H]⁺

¹H-NMR (400 MHz, DMSO-d6): δ [ppm]=1.68 (d, 3H), 2.07-2.24 (m, 2H), 2.55-2.62 (m, 1H), 2.69-2.77 (m, 1H), 3.20-3.50 (m, 4H), 4.17-4.25 (m, 2H), 6.19 (q, 1H), 6.70 (2s, 1H), 7.84 (s, 1H), 7.87 (s, 1H), 7.93 (br dd, 1H), 8.32 (br d, 2H), 8.56 (br s, 1H), 8.80 (d, 1H), 9.12 (s, 1H), 9.71-9.92 (m, 2H).

### Intermediate 282

### 5-[5′,6′-dihydrospiro[pyrrolidine-3,4′-pyrrolo[1,2-b]pyrazol]-2′-yl]-3-[(1S)-1-(pyridin-3-yl)ethoxy]pyridin-2-amine-hydrochloride salt

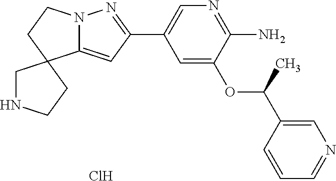 [SMILES: C[C@H](Oc1cc(-c2cc3n(n2)CCC32CCNC2)cnc1N)c1cccnc1 ; Cl]

Intermediate 282 was prepared in analogy to Intermediate 279 using (rac)-tert-butyl-2′-{6-amino-5-[(1S)-1-(pyridin-3-yl)ethoxy]pyridin-3-yl}-5′,6′-dihydrospiro[pyrrolidine-3,4′-pyrrolo[1,2-b]pyrazole]-1-carboxylate (60.0 mg, 126 μmol).

LC-MS (method 1): Rt=0.77 min; MS (ESIpos): m/z=377 [M+H]⁺

¹H-NMR (400 MHz, DMSO-d6): δ [ppm]=1.67 (d, 3H), 2.09-2.25 (m, 2H), 2.53-2.62 (m, 1H), 2.67-2.77 (m, 1H), 3.19-3.48 (m, 4H), 4.14-4.27 (m, 2H), 6.13 (q, 1H), 6.68 (2s, 1H), 7.75-7.83 (m, 2H), 7.85 (s, 1H), 8.26 (br s, 2H), 8.40 (br d, 1H), 8.72 (br d, 1H), 9.02 (s, 1H), 9.63-9.84 (m, 2H).

### Intermediate 283

### 5-[5′,6′-dihydrospiro[pyrrolidine-3,4′-pyrrolo[1,2-b]pyrazol]-2′-yl]-3-[(1R)-1-(pyridin-2-yl)ethoxy]pyridin-2-amine-hydrochloride salt

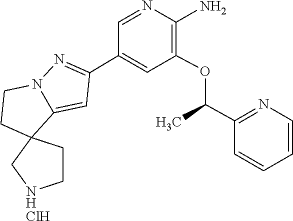 [SMILES: C[C@@H](Oc1cc(-c2cc3n(n2)CCC32CCNC2)cnc1N)c1ccccn1 ; Cl]

Intermediate 283 was prepared in analogy to Intermediate 279 using (rac)-tert-butyl-2′-{6-amino-5-[(1R)-1-(pyridin-2-yl)ethoxy]pyridin-3-yl}-5′,6′-dihydrospiro[pyrrolidine-3,4′-pyrrolo[1,2-b]pyrazole]-1-carboxylate (238 mg, 499 μmol).

LC-MS (method 1): Rₜ=0.79 min; MS (ESIpos): m/z=378 [M+H]⁺

¹H-NMR (400 MHz, DMSO-d6) δ[ppm]: 1.065 (0.61), 1.593 (16.00), 1.688 (2.46), 1.705 (2.44), 2.144 (0.41), 2.323 (0.43), 2.327 (0.63), 2.332 (0.47), 2.518 (2.08), 2.523 (1.47), 2.665 (0.50), 2.669 (0.72), 2.673 (0.67), 3.384 (0.72), 3.396 (0.48), 3.551 (0.52), 3.941 (2.69), 4.184 (0.57), 6.604 (2.46), 7.618 (0.50), 7.641 (1.19), 7.826 (1.15), 7.922 (0.43), 8.604 (0.54), 8.616 (0.49).

### Intermediate 284

### 5-[5′,6′-dihydrospiro[pyrrolidine-3,4′-pyrrolo[1,2-b]pyrazol]-2′-yl]-3-[(1S)-1-(pyridin-2-yl)ethoxy]pyridin-2-amine-hydrochloride salt

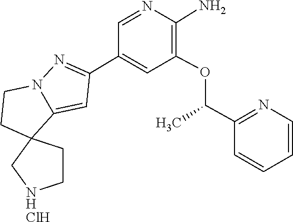 [SMILES: C[C@H](Oc1cc(-c2cc3n(n2)CCC32CCNC2)cnc1N)c1ccccn1 ; Cl]

Intermediate 284 was prepared in analogy to Intermediate 279 using (rac)-tert-butyl-2′-{6-amino-5-[(1S)-1-(pyridin-2-yl)ethoxy]pyridin-3-yl}-5′,6′-dihydrospiro[pyrrolidine-3,4′-pyrrolo[1,2-b]pyrazole]-1-carboxylate (335 mg, 93% purity, 654 μmol).

LC-MS (method 1): Rₜ=0.79 min; MS (ESIpos): m/z=378 [M+H]⁺

¹H-NMR (400 MHz, DMSO-d6) δ[ppm]: 1.593 (16.00), 1.663 (0.65), 1.679 (0.90), 1.688 (3.30), 1.703 (3.30), 2.133 (0.49), 2.143 (0.64), 2.153 (0.58), 2.323 (0.74), 2.327 (1.04), 2.331 (0.78), 2.518 (6.25), 2.523 (4.40), 2.558 (0.58), 2.574 (0.51), 2.665 (0.81), 2.669 (1.27), 3.384 (0.81), 3.396 (0.72), 3.730 (0.41), 3.914 (4.94), 4.184 (0.85), 5.841 (0.58), 5.857 (0.57), 6.600 (3.18), 7.384 (0.48), 7.400 (0.60), 7.415 (0.55), 7.608 (0.72), 7.627 (0.92), 7.638 (1.45), 7.823 (1.57), 7.910 (0.67), 8.599 (0.85), 8.611 (0.76).

### Intermediate 285

### (rac)-5-[5′,6′-dihydrospiro[pyrrolidine-3,4′-pyrrolo[1,2-b]pyrazol]-2′-yl]-3-[(trifluoromethyl)sulfanyl]pyridin-2-amine-hydrochloride salt

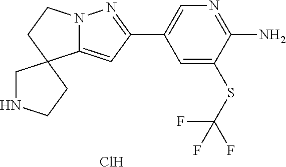 [SMILES: Nc1ncc(-c2cc3n(n2)CCC32CCNC2)cc1SC(F)(F)F ; Cl]

Intermediate 285 was prepared in analogy to Intermediate 279 using (rac)-tert-butyl-2′-{6-amino-5-[(trifluoromethyl)sulfanyl]pyridin-3-yl}-5′,6′-dihydrospiro[pyrrolidine-3,4′-pyrrolo[1,2-b]pyrazole]-1-carboxylate (100 mg, 220 μmol).

LC-MS (method 1): Rt=0.93 min; MS (ESIpos): m/z=356 [M+H]⁺

### Intermediate 286

### (rac)-5-[5′,6′-dihydrospiro[pyrrolidine-3,4′-pyrrolo[1,2-b]pyrazol]-2′-yl]-3-[(2-fluorophenyl)methoxy]pyridin-2-amine-hydrochloride salt

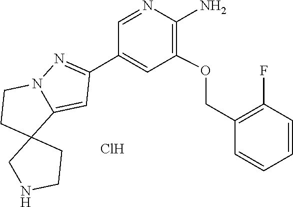 [SMILES: Nc1ncc(-c2cc3n(n2)CCC32CCNC2)cc1OCc1ccccc1F ; Cl]

Intermediate 286 was prepared in analogy to Intermediate 279 using (rac)-tert-butyl-2′-{6-amino-5-[(2-fluorophenyl)methoxy]pyridin-3-yl}-5′,6′-dihydrospiro[pyrrolidine-3,4′-pyrrolo[1,2-b]pyrazole]-1-carboxylate (488 mg, 1.02 mmol).

LC-MS (method 1): Rt=0.95 min; MS (ESIpos): m/z=380 [M+H]⁺

### Intermediate 287

### (rac)-5-[5′,6′-dihydrospiro[pyrrolidine-3,4′-pyrrolo[1,2-b]pyrazol]-2′-yl]-3-[(3-fluorophenyl)methoxy]pyridin-2-amine-hydrochloride salt

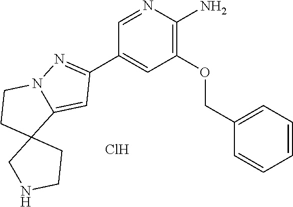 [SMILES: Nc1ncc(-c2cc3n(n2)CCC32CCNC2)cc1OCc1ccccc1 ; Cl]

Intermediate 287 was prepared in analogy to Intermediate 279 using (rac)-tert-butyl-2′-{6-amino-5-[(3-fluorophenyl)methoxy]pyridin-3-yl}-5′,6′-dihydrospiro[pyrrolidine-3,4′-pyrrolo[1,2-b]pyrazole]-1-carboxylate (440 mg, 918 μmol).

LC-MS (method 1): Rt=0.95 min; MS (ESIpos): m/z=380 [M+H]⁺

### Intermediate 288

### (rac)-3-[(2,6-dichlorophenyl)methoxy]-5-[5′,6′-dihydrospiro[pyrrolidine-3,4′-pyrrolo[1,2-b]pyrazol]-2′-yl]pyridin-2-amine-hydrochloride salt

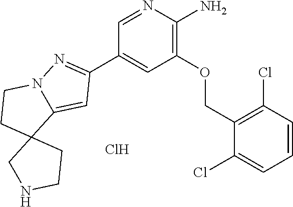 [SMILES: Nc1ncc(-c2cc3n(n2)CCC32CCNC2)cc1OCc1c(Cl)cccc1Cl ; Cl]

Intermediate 288 was prepared in analogy to Intermediate 279 using (rac)-tert-butyl-2′-{6-amino-5-[(2,6-dichlorophenyl)methoxy]pyridin-3-yl}-5′,6′-dihydrospiro[pyrrolidine-3,4′-pyrrolo[1,2-b]pyrazole]-1-carboxylate (70.0 mg, 132 μmol).

LC-MS (method 1): Rt=1.04 min; MS (ESIpos): m/z=430 [M+H]⁺

### Intermediate 289

### (rac)-3-[(2-chlorophenyl)methoxy]-5-[5′,6′-dihydrospiro[pyrrolidine-3,4′-pyrrolo[1,2-b]pyrazol]-2′-yl]pyridin-2-amine-hydrochloride salt

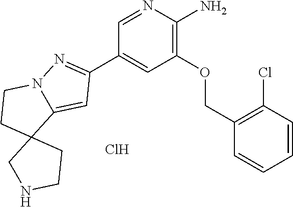 [SMILES: Nc1ncc(-c2cc3n(n2)CCC32CCNC2)cc1OCc1ccccc1Cl ; Cl]

Intermediate 289 was prepared in analogy to Intermediate 279 using (rac)-tert-butyl-2′-{6-amino-5-[(2-chlorophenyl)methoxy]pyridin-3-yl}-5′,6′-dihydrospiro[pyrrolidine-3,4′-pyrrolo[1,2-b]pyrazole]-1-carboxylate (188 mg, 379 μmol).

LC-MS (method 1): Rt=0.99 min; MS (ESIpos): m/z=396 [M+H]⁺

### Intermediate 290

### (rac)-5-[5′,6′-dihydrospiro[pyrrolidine-3,4′-pyrrolo[1,2-b]pyrazol]-2′-yl]-3-[(pyridin-2-yl)methoxy]pyridin-2-amine-hydrochloride salt

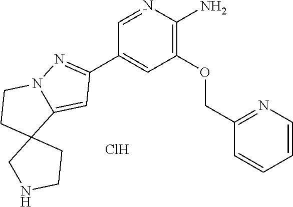 [SMILES: Nc1ncc(-c2cc3n(n2)CCC32CCNC2)cc1OCc1ccccn1 ; Cl]

Intermediate 290 was prepared in analogy to Intermediate 279 using (rac)-tert-butyl-2′-{6-amino-5-[(pyridin-2-yl)methoxy]pyridin-3-yl}-5′,6′-dihydrospiro[pyrrolidine-3,4′-pyrrolo[1,2-b]pyrazole]-1-carboxylate (310 mg, 670 μmol).

LC-MS (method 1): Rt=0.76 min; MS (ESIpos): m/z=363 [M+H]⁺

### Intermediate 291

### (rac)-3-[(2-chloro-5-fluorophenyl)methoxy]-5-[5′,6′-dihydrospiro[pyrrolidine-3,4′-pyrrolo[1,2-b]pyrazol]-2′-yl]pyridin-2-amine-hydrochloride salt

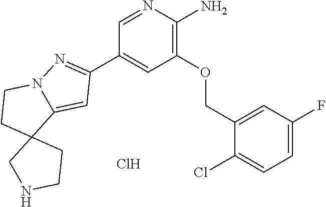 [SMILES: Nc1ncc(-c2cc3n(n2)CCC32CCNC2)cc1OCc1cc(F)ccc1Cl ; Cl]

Intermediate 291 was prepared in analogy to Intermediate 279 using (rac)-tert-butyl-2′-{6-amino-5-[(2-chloro-5-fluorophenyl)methoxy]pyridin-3-yl}-5′,6′-dihydrospiro[pyrrolidine-3,4′-pyrrolo[1,2-b]pyrazole]-1-carboxylate (130 mg, 253 μmol).

LC-MS (method 1): Rt=1.01 min; MS (ESIpos): m/z=414 [M+H]⁺

### Intermediate 292

### (rac)-3-(benzyloxy)-5-[5′,6′-dihydrospiro[pyrrolidine-3,4′-pyrrolo[1,2-b]pyrazol]-2′-yl]pyridin-2-amine-hydrochloride salt

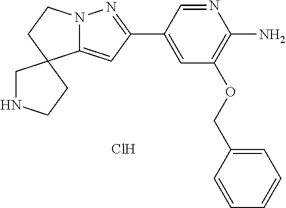 [SMILES: Nc1ncc(-c2cc3n(n2)CCC32CCNC2)cc1OCc1ccccc1 ; Cl]

Intermediate 292 was prepared in analogy to Intermediate 279 using (rac)-tert-butyl-2′-[6-amino-5-(benzyloxy)pyridin-3-yl]-5′,6′-dihydrospiro[pyrrolidine-3,4′-pyrrolo[1,2-b]pyrazole]-1-carboxylate (78.0 mg, 169 μmol).

LC-MS (method 1): Rt=0.95 min; MS (ESIpos): m/z=362 [M+H]⁺

### Intermediate 293

### (rac)-5-[5′,6′-dihydrospiro[pyrrolidine-3,4′-pyrrolo[1,2-b]pyrazol]-2′-yl]-1′-methyl-1′,2′,3′,6′-tetrahydro[3,4′-bipyridin]-2-amine-hydrochloride salt

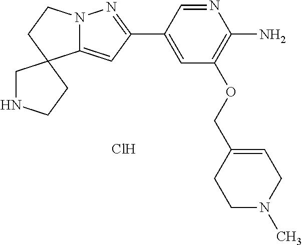 [SMILES: CN1CC=C(COc2cc(-c3cc4n(n3)CCC43CCNC3)cnc2N)CC1 ; Cl]

Intermediate 293 was prepared in analogy to Intermediate 279 using (rac)-tert-butyl-2′-(2-amino-1′-methyl-1′,2′,3′,6′-tetrahydro[3,4′-bipyridin]-5-yl)-5′,6′-dihydrospiro[pyrrolidine-3,4′-pyrrolo[1,2-b]pyrazole]-1-carboxylate (80.0 mg, 80% purity, 142 μmol).

LC-MS (method 1): Rₜ=0.70 min; MS (ESIpos): m/z=352 [M+H]⁺

¹H-NMR (400 MHz, DMSO-d6) δ[ppm]: 1.064 (16.00), 1.154 (0.48), 1.291 (1.14), 2.158 (0.55), 2.178 (0.60), 2.522 (2.97), 2.879 (2.00), 2.891 (1.80), 3.255 (0.42), 3.269 (0.51), 3.284 (0.63), 3.383 (2.49), 3.564 (1.08), 4.212 (0.54), 4.223 (0.49), 5.944 (0.48), 6.687 (2.26), 8.070 (0.93), 8.075 (1.01), 8.259 (1.08), 8.264 (1.01).

### Intermediate 294

### 5-[5′,6′-dihydrospiro[pyrrolidine-3,4′-pyrrolo[1,2-b]pyrazol]-2′-yl]-3-[(1R)-1-(pyridin-4-yl)ethoxy]pyridin-2-amine

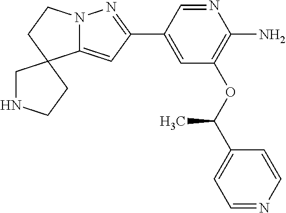 [SMILES: C[C@@H](Oc1cc(-c2cc3n(n2)CCC32CCNC2)cnc1N)c1ccncc1]

Intermediate 294 was prepared in analogy to Intermediate 279 using (rac)-tert-butyl-2′-{6-amino-5-[(1R)-1-(pyridin-4-yl)ethoxy]pyridin-3-yl}-5′,6′-dihydrospiro[pyrrolidine-3,4′-pyrrolo[1,2-b]pyrazole]-1-carboxylate (220 mg, 462 μmol).

LC-MS (method 1): Rₜ=0.71 min; MS (ESIpos): m/z=377 [M+H]⁺

¹H-NMR (400 MHz, DMSO-d6) δ[ppm]: 1.064 (16.00), 1.383 (0.68), 1.399 (0.68), 1.592 (11.43), 1.639 (0.70), 1.648 (1.00), 1.655 (0.81), 1.663 (0.96), 2.518 (2.55), 2.522 (1.59), 3.383 (0.48), 6.658 (0.86), 6.660 (0.81), 7.728 (0.47), 7.879 (0.58), 8.853 (0.55), 8.869 (0.89), 8.882 (0.45).

### Intermediate 295

### 5-[5′,6′-dihydrospiro[pyrrolidine-3,4′-pyrrolo[1,2-b]pyrazol]-2′-yl]-3-[(1S)-1-phenylethoxy]pyridin-2-amine-hydrochloride salt

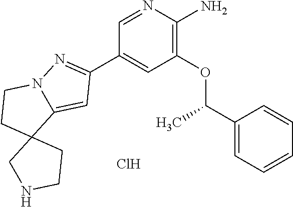 [SMILES: C[C@H](Oc1cc(-c2cc3n(n2)CCC32CCNC2)cnc1N)c1ccccc1 ; Cl]

Intermediate 295 was prepared in analogy to Intermediate 279 using (rac)-tert-butyl-2′-{6-amino-5-[(1S)-1-phenylethoxy]pyridin-3-yl}-5′,6′-dihydrospiro[pyrrolidine-3,4′-pyrrolo[1,2-b]pyrazole]-1-carboxylate (80.0 mg, 168 μmol).

LC-MS (method 1): Rₜ=0.96 min; MS (ESIpos): m/z=376 [M+H]⁺

### Intermediate 296

### 5-[5′,6′-dihydrospiro[pyrrolidine-3,4′-pyrrolo[1,2-b]pyrazol]-2′-yl]-3-[(1R)-1-phenylethoxy]pyridin-2-amine-hydrochloride salt

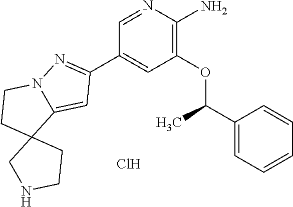 [SMILES: C[C@@H](Oc1cc(-c2cc3n(n2)CCC32CCNC2)cnc1N)c1ccccc1 ; Cl]

Intermediate 296 was prepared in analogy to Intermediate 279 using (rac)-tert-butyl-2′-{6-amino-5-[(1R)-1-phenylethoxy]pyridin-3-yl}-5′,6′-dihydrospiro[pyrrolidine-3,4′-pyrrolo[1,2-b]pyrazole]-1-carboxylate (140 mg, 294 μmol).

LC-MS (method 1): Rt=0.95 min; MS (ESIpos): m/z=376 [M+H]⁺

¹H-NMR (400 MHz, DMSO-d6) δ[ppm]: 1.065 (1.41), 1.638 (0.50), 1.654 (0.48), 2.327 (0.48), 2.518 (1.92), 2.523 (1.23), 2.665 (0.45), 2.669 (0.56), 3.566 (16.00), 3.580 (3.69), 6.597 (0.84).

### Intermediate 297

### 5-[(3S)-5′,6′-dihydrospiro[pyrrolidine-3,4′-pyrrolo[1,2-b]pyrazol]-2′-yl]-3-(trifluoromethyl)pyridin-2-amine

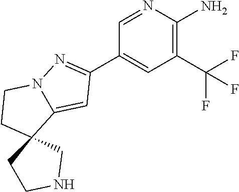 [SMILES: Nc1ncc(-c2cc3n(n2)CC[C@]32CCNC2)cc1C(F)(F)F]

tert-butyl-(3S)-2′-[6-amino-5-(trifluoromethyl)pyridin-3-yl]-5′,6′-dihydrospiro[pyrrolidine-3,4′-pyrrolo[1,2-b]pyrazole]-1-carboxylate (290 mg, 685 μmol) was stirred in TFE (7.0 mL, 96 mmol) at 130° C. in the microwave for 16 h. The mixture was evaporated and purified by preparative HPLC to give 55.0 mg (95% purity, 24% yield) of the title compound.

1H NMR (DMSO-d6) δ: 8.57 (d, 1H), 8.00 (d, 1H), 6.52 (s, 2H), 6.46 (s, 1H), 4.09-4.16 (m, 2H), 2.94-3.00 (m, 2H), 2.78-2.90 (m, 2H), 2.39-2.49 (m, 2H), 1.84-2.00 (m, 2H)

[α]²⁰_{D}: −11.1° (c=1.00, MeOH)

LC-MS (method 1): Rₜ=0.85 min; MS (ESIpos): m/z=324 [M+H]⁺

### Intermediate 298

### 5-[(3R)-5′,6′-dihydrospiro[pyrrolidine-3,4′-pyrrolo[1,2-b]pyrazol]-2′-yl]-3-(trifluoromethyl)pyridin-2-amine

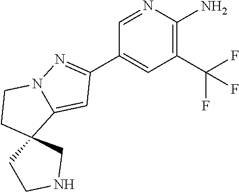 [SMILES: Nc1ncc(-c2cc3n(n2)CC[C@@]32CCNC2)cc1C(F)(F)F]

Intermediate 298 was prepared in analogy to Intermediate 297 using tert-butyl-(3R)-2′-[6-amino-5-(trifluoromethyl)pyridin-3-yl]-5′,6′-dihydrospiro[pyrrolidine-3,4′-pyrrolo[1,2-b]pyrazole]-1-carboxylate (100 mg, 236 μmol).

1H NMR (DMSO-d6) δ: 8.57 (d, 1H), 8.00 (d, 1H), 6.52 (s, 2H), 6.46 (s, 1H), 4.06-4.23 (m, 2H), 2.91-3.03 (m, 2H), 2.77-2.90 (m, 2H), 1.84-2.00 (m, 2H)

[α]²⁰_{D}: +14.3° (c=1.00, MeOH)

LC-MS (method 1): Rₜ=0.83 min; MS (ESIpos): m/z=324 [M+H]⁺

### Intermediate 299

### 5-[(3S)-5′,6′-dihydrospiro[pyrrolidine-3,4′-pyrrolo[1,2-b]pyrazol]-2′-yl]-3-(trifluoromethyl)pyridin-2-amine-hydrochloride salt

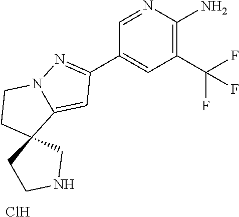 [SMILES: Nc1ncc(-c2cc3n(n2)CC[C@]32CCNC2)cc1C(F)(F)F ; Cl]

Intermediate 299 was prepared in analogy to Intermediate 279 using tert-butyl-(3S)-2′-[6-amino-5-(trifluoromethyl)pyridin-3-yl]-5′,6′-dihydrospiro[pyrrolidine-3,4′-pyrrolo[1,2-b]pyrazole]-1-carboxylate (7.96 g, 18.8 mmol, stereoisomer 2).

LC-MS (method 1): Rₜ=0.84 min; MS (ESIpos): m/z=325 [M+H]⁺

### Intermediate 300

### 5-[(3S)-5′,6′-dihydrospiro[pyrrolidine-3,4′-pyrrolo[1,2-b]pyrazol]-2′-yl]-3-(pyridazin-3-ylmethoxy)pyridin-2-amine-hydrochloride salt

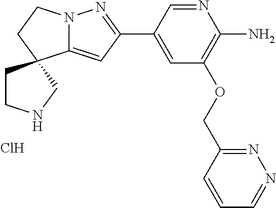 [SMILES: Cl ; Nc1ncc(-c2cc3n(n2)CC[C@]32CCNC2)cc1OCc1cccnn1]

Intermediate 300 was prepared in analogy to Intermediate 279 using tert-butyl-(3S)-2′-{6-amino-5-[(pyridazin-3-yl)methoxy]pyridin-3-yl}-5′,6′-dihydrospiro[pyrrolidine-3,4′-pyrrolo[1,2-b]pyrazole]-1-carboxylate (98.0 mg, 211 μmol, stereoisomer 1).

LC-MS (method 1): Rt=0.64 min; MS (ESIpos): m/z=364 [M+H]⁺

### Intermediate 301

### 5-[(3R)-5′,6′-dihydrospiro[pyrrolidine-3,4′-pyrrolo[1,2-b]pyrazol]-2′-yl]-3-(pyridazin-3-ylmethoxy)pyridin-2-amine-hydrochloride salt

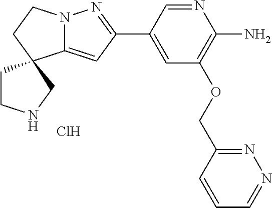 [SMILES: Nc1ncc(-c2cc3n(n2)CC[C@@]32CCNC2)cc1OCc1cccnn1 ; Cl]

Intermediate 301 was prepared in analogy to Intermediate 279 using tert-butyl-(3R)-2′-{6-amino-5-[(pyridazin-3-yl)methoxy]pyridin-3-yl}-5′,6′-dihydrospiro[pyrrolidine-3,4′-pyrrolo[1,2-b]pyrazole]-1-carboxylate (105 mg, 227 μmol, stereoisomer 2).

LC-MS (method 1): Rt=0.64 min; MS (ESIpos): m/z=364 [M+H]⁺

### Intermediate 302

### 5-[(3S)-5′,6′-dihydrospiro[pyrrolidine-3,4′-pyrrolo[1,2-b]pyrazol]-2′-yl]-3-[1-(pyrimidin-5-yl)ethoxy]pyridin-2-amine hydrochloride (Isomer 1)

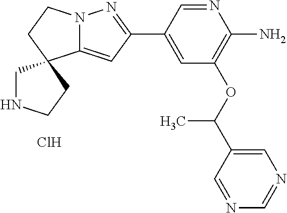 [SMILES: CC(Oc1cc(-c2cc3n(n2)CC[C@]32CCNC2)cnc1N)c1cncnc1 ; Cl]

Intermediate 302 was prepared in analogy to Intermediate 279 using tert-butyl-2′-(6-amino-5-{[1-(pyrimidin-5-yl)ethyl]oxy}pyridin-3-yl)-5′,6′-dihydrospiro[pyrrolidine-3,4′-pyrrolo[1,2-b]pyrazole]-1-carboxylate (84.0 mg, 176 μmol, isomer 1).

LC-MS (method 1): Rt=0.69 min; MS (ESIpos): m/z=378 [M+H]⁺

### Intermediate 303

### 5-[(S)-5′,6′-dihydrospiro[pyrrolidine-3,4′-pyrrolo[1,2-b]pyrazol]-2′-yl]-3-{[1-(pyrimidin-5-yl)ethyl]oxy}pyridin-2-amine-hydrochloride salt (Isomer 2)

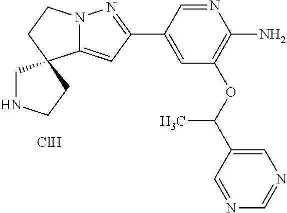 [SMILES: CC(Oc1cc(-c2cc3n(n2)CC[C@]32CCNC2)cnc1N)c1cncnc1 ; Cl]

Intermediate 303 was prepared in analogy to Intermediate 279 using tert-butyl-2′-(6-amino-5-{[1-(pyrimidin-5-yl)ethyl]oxy}pyridin-3-yl)-5′,6′-dihydrospiro[pyrrolidine-3,4′-pyrrolo[1,2-b]pyrazole]-1-carboxylate (104 mg, 218 μmol, isomer 3).

[α]²⁰_{D}: +5.7° (c=1.00, DMSO)

LC-MS (method 1): Rt=0.69 min; MS (ESIpos): m/z=378 [M+H]⁺

### Intermediate 304

### 3-{[1-(5-chloro-3-fluoropyridin-2-yl)ethyl]oxy}-5-[5′,6′-dihydrospiro[pyrrolidine-3,4′-pyrrolo[1,2-b]pyrazol]-2′-yl]pyridin-2-amine-hydrochloride salt (Diastereomeric Mixture)

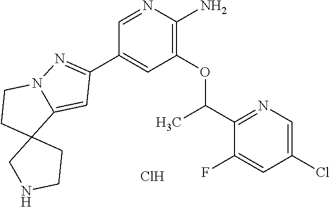 [SMILES: CC(Oc1cc(-c2cc3n(n2)CCC32CCNC2)cnc1N)c1ncc(Cl)cc1F ; Cl]

Intermediate 304 was prepared in analogy to Intermediate 279 using tert-butyl-2′-(6-amino-5-{[1-(5-chloro-3-fluoropyridin-2-yl)ethyl]oxy}pyridin-3-yl)-5′,6′-dihydrospiro[pyrrolidine-3,4′-pyrrolo[1,2-b]pyrazole]-1-carboxylate (diastereomeric mixture) (65.0 mg, 123 μmol).

LC-MS (method 1): Rt=0.96 min; MS (ESIpos): m/z=429 [M+H]⁺

### Intermediate 305

### 5-(5′,6′-dihydrospiro[azetidine-3,4′-pyrrolo[1,2-b]pyrazol]-2′-yl)-3-(trifluoromethyl)pyridin-2-amine

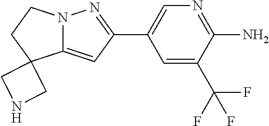 [SMILES: Nc1ncc(-c2cc3n(n2)CCC32CNC2)cc1C(F)(F)F]

tert-butyl 2′-[6-amino-5-(trifluoromethyl)pyridin-3-yl]-5′,6′-dihydrospiro[azetidine-3,4′-pyrrolo[1,2-b]pyrazole]-1-carboxylate (2.10 g, 5.13 mmol) was solubilised in 1,4-dioxane (45 mL) and HCl (13 mL, 4.0 M in 1,4-dioxane, 51 mmol) was added. The mixture was stirred overnight at rt. It was concentrated under reduced pressure to give 2.2 g of the title compound.

LC-MS (method 1): Rt=0.81 min; MS (ESIpos): m/z=310 [M+H]⁺

### Intermediate 306

### 2-amino-5-(5′,6′-dihydrospiro[azetidine-3,4′-pyrrolo[1,2-b]pyrazol]-2′-yl)pyridine-3-carbonitrile-hydrochloride salt

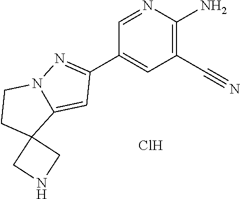 [SMILES: N#Cc1cc(-c2cc3n(n2)CCC32CNC2)cnc1N ; Cl]

Intermediate 306 was prepared in analogy to Intermediate 305 using tert-butyl 2′-(6-amino-5-cyanopyridin-3-yl)-5′,6′-dihydrospiro[azetidine-3,4′-pyrrolo[1,2-b]pyrazole]-1-carboxylate (2.60 g, 7.10 mmol).

LC-MS (method 1): Rt=0.66 min; MS (ESIpos): m/z=267 [M+H]⁺

¹H-NMR (400 MHz, DMSO-d6) δ[ppm]: 1.064 (9.31), 1.154 (7.68), 1.230 (0.76), 1.248 (0.74), 1.269 (2.13), 1.351 (0.40), 2.297 (0.91), 2.523 (7.54), 2.934 (3.74), 2.952 (6.03), 2.968 (4.00), 3.247 (0.64), 3.383 (0.92), 3.676 (0.46), 3.700 (0.47), 4.032 (2.51), 4.088 (2.88), 4.106 (3.92), 4.123 (6.34), 4.140 (4.27), 4.157 (5.70), 4.174 (10.27), 4.188 (5.13), 4.403 (0.45), 4.785 (3.36), 6.625 (2.98), 6.780 (16.00), 7.526 (0.82), 7.553 (0.84), 8.137 (0.46), 8.185 (7.99), 8.191 (7.86), 8.201 (1.88), 8.207 (1.84), 8.245 (0.97), 8.603 (8.32), 8.609 (8.25), 8.617 (2.05), 8.622 (1.85), 9.343 (1.15).

### Intermediate 307

### 3-(difluoromethoxy)-5-(5′,6′-dihydrospiro[azetidine-3,4′-pyrrolo[1,2-b]pyrazol]-2′-yl)pyridin-2-amine-hydrochloride salt

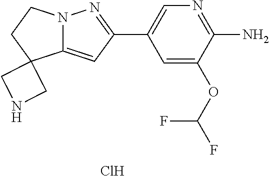 [SMILES: Nc1ncc(-c2cc3n(n2)CCC32CNC2)cc1OC(F)F ; Cl]

Intermediate 307 was prepared in analogy to Intermediate 305 using tert-butyl 2′-[6-amino-5-(difluoromethoxy)pyridin-3-yl]-5′,6′-dihydrospiro[azetidine-3,4′-pyrrolo[1,2-b]pyrazole]-1-carboxylate (3.40 g, 8.35 mmol).

LC-MS (method 1): Rt=0.72 min; MS (ESIpos): m/z=308 [M+H]⁺

### Intermediate 308

### 5-(5′,6′-dihydrospiro[azetidine-3,4′-pyrrolo[1,2-b]pyrazol]-2′-yl)-3-(trifluoromethoxy)pyridin-2-amine-hydrochloride salt

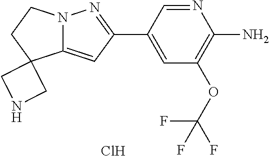 [SMILES: Nc1ncc(-c2cc3n(n2)CCC32CNC2)cc1OC(F)(F)F ; Cl]

Intermediate 308 was prepared in analogy to Intermediate 305 using tert-butyl 2′-[6-amino-5-(trifluoromethoxy)pyridin-3-yl]-5′,6′-dihydrospiro[azetidine-3,4′-pyrrolo[1,2-b]pyrazole]-1-carboxylate (1.40 g, 3.29 mmol).

LC-MS (method 1): Rt=0.82 min; MS (ESIpos): m/z=326 [M+H]⁺

¹H-NMR (400 MHz, DMSO-d6) δ[ppm]: 1.064 (16.00), 1.118 (0.84), 1.123 (0.86), 2.518 (10.94), 2.523 (7.50), 2.931 (1.49), 2.948 (2.41), 2.966 (1.62), 3.384 (1.06), 4.030 (0.92), 4.112 (1.58), 4.130 (2.52), 4.147 (1.63), 4.165 (2.49), 4.181 (4.28), 4.197 (2.45), 5.003 (0.61), 6.647 (0.85), 6.811 (4.35), 7.917 (1.40), 8.342 (3.33), 8.347 (3.44), 8.353 (0.77), 9.326 (0.79).

### Intermediate 309

### 5-(5′,6′-dihydrospiro[azetidine-3,4′-pyrrolo[1,2-b]pyrazol]-2′-yl)-3-[(1S)-1-(pyridin-2-yl)ethoxy]pyridin-2-amine-hydrochloride salt

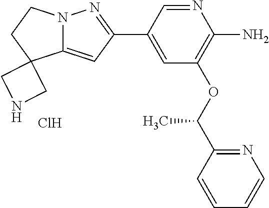 [SMILES: C[C@H](Oc1cc(-c2cc3n(n2)CCC32CNC2)cnc1N)c1ccccn1 ; Cl]

Intermediate 309 was prepared in analogy to Intermediate 305 using tert-butyl 2′-{6-amino-5-[(1S)-1-(pyridin-2-yl)ethoxy]pyridin-3-yl}-5′,6′-dihydrospiro[azetidine-3,4′-pyrrolo[1,2-b]pyrazole]-1-carboxylate (31.6 mg, 68.3 μmol).

¹H-NMR (400 MHz, DMSO-d6): δ [ppm]=1.70 (d, 3H), 2.94 (t, 2H), 5.90 (q, 1H), 6.79 (s, 1H), 7.39-7.50 (m, 1H), 7.62-7.70 (m, 2H), 7.87 (d, 1H), 7.96 (br t, 1H), 8.14-8.49 (m, 2H), 8.64 (d, 1H), 9.38-9.66 (m, 2H), 13.40-14.82 (m, 1H).

LC-MS (method 1): Rₜ=0.78 min; MS (ESIpos): m/z=363 [M+H]⁺

### Intermediate 310

### 5-(5′,6′-dihydrospiro[azetidine-3,4′-pyrrolo[1,2-b]pyrazol]-2′-yl)-3-[(1R)-1-(pyridin-2-yl)ethoxy]pyridin-2-amine-hydrochloride salt

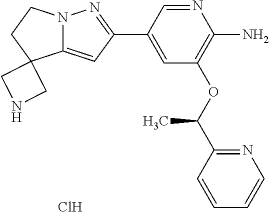 [SMILES: C[C@@H](Oc1cc(-c2cc3n(n2)CCC32CNC2)cnc1N)c1ccccn1 ; Cl]

Intermediate 310 was prepared in analogy to Intermediate 305 using tert-butyl 2′-{6-amino-5-[(1R)-1-(pyridin-2-yl)ethoxy]pyridin-3-yl}-5′,6′-dihydrospiro[azetidine-3,4′-pyrrolo[1,2-b]pyrazole]-1-carboxylate (100 mg, 70% purity, 151 μmol).

LC-MS (method 1): Rₜ=0.78 min; MS (ESIpos): m/z=363 [M+H]⁺

¹H-NMR (400 MHz, DMSO-d6) δ[ppm]: 1.065 (16.00), 1.238 (2.15), 1.251 (2.00), 1.648 (0.67), 1.664 (0.68), 1.687 (1.04), 1.703 (1.01), 1.907 (0.89), 2.331 (2.28), 2.336 (1.00), 2.518 (14.04), 2.523 (9.58), 2.673 (2.29), 2.678 (1.04), 2.928 (0.46), 3.385 (0.62), 3.566 (6.08), 4.109 (0.49), 4.156 (0.50), 4.168 (0.58), 6.718 (0.86), 7.568 (0.49), 7.832 (0.69), 7.849 (0.46), 8.571 (0.42), 8.580 (0.42).

### Intermediate 311

### 5-(5′,6′-dihydrospiro[azetidine-3,4′-pyrrolo[1,2-b]pyrazol]-2′-yl)-3-[(1R)-1-phenylethoxy]pyridin-2-amine-hydrochloride salt

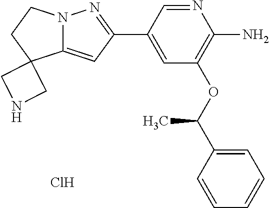 [SMILES: C[C@@H](Oc1cc(-c2cc3n(n2)CCC32CNC2)cnc1N)c1ccccc1 ; Cl]

Intermediate 311 was prepared in analogy to Intermediate 305 using tert-butyl 2′-{6-amino-5-[(1R)-1-phenylethoxy]pyridin-3-yl}-5′,6′-dihydrospiro[azetidine-3,4′-pyrrolo[1,2-b]pyrazole]-1-carboxylate (300 mg, 70% purity, 455 μmol).

LC-MS (method 1): Rₜ=0.96 min; MS (ESIpos): m/z=362 [M+H]⁺

¹H-NMR (400 MHz, DMSO-d6) δ[ppm]: 1.065 (0.79), 1.156 (10.27), 1.232 (0.88), 1.256 (0.46), 1.642 (1.09), 1.657 (1.05), 1.780 (3.25), 1.797 (3.19), 1.907 (0.70), 2.327 (3.04), 2.331 (2.21), 2.518 (16.00), 2.523 (10.08), 2.669 (3.04), 2.673 (2.19), 2.939 (0.92), 3.252 (0.47), 3.385 (0.96), 3.457 (0.82), 3.470 (1.04), 3.487 (1.23), 3.499 (1.38), 3.663 (1.07), 3.667 (0.94), 3.677 (1.03), 3.699 (0.84), 3.709 (0.64), 3.714 (0.71), 3.725 (0.43), 3.729 (0.42), 4.005 (0.62), 4.025 (0.68), 4.094 (0.64), 4.113 (0.99), 4.137 (0.90), 4.153 (0.93), 4.174 (1.20), 4.189 (1.83), 4.205 (1.17), 5.336 (0.52), 5.353 (0.50), 5.760 (2.62), 6.602 (0.83), 6.652 (1.21), 6.656 (0.40), 6.744 (0.98), 6.784 (1.45), 7.286 (0.49), 7.303 (0.53), 7.321 (0.58), 7.339 (0.57), 7.355 (0.65), 7.362 (0.77), 7.375 (1.08), 7.381 (1.23), 7.393 (0.64), 7.398 (0.59), 7.467 (1.09), 7.486 (1.40), 7.489 (1.46), 7.507 (0.75), 7.619 (0.87), 7.775 (0.65), 7.803 (0.51), 7.958 (0.52), 8.134 (0.61), 11.749 (0.45).

### Intermediate 312

### 5-(5′,6′-dihydrospiro[azetidine-3,4′-pyrrolo[1,2-b]pyrazol]-2′-yl)-3-[(1S)-1-phenylethoxy]pyridin-2-amine-hydrochloride salt

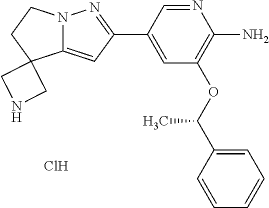 [SMILES: C[C@H](Oc1cc(-c2cc3n(n2)CCC32CNC2)cnc1N)c1ccccc1 ; Cl]

Intermediate 312 was prepared in analogy to Intermediate 305 using tert-butyl 2′-{6-amino-5-[(1S)-1-phenylethoxy]pyridin-3-yl}-5′,6′-dihydrospiro[azetidine-3,4′-pyrrolo[1,2-b]pyrazole]-1-carboxylate (110 mg, 80% purity, 191 μmol).

LC-MS (method 1): Rₜ=1.02 min; MS (ESIpos): m/z=398 [M+H]⁺

### Intermediate 313

### (rac)-5-[6,7-dihydrospiro[pyrazolo[5,1-c][1,4]oxazine-4,3′-pyrrolidin]-2-yl]-3-(trifluoromethoxy)pyridin-2-amine-hydrochloride salt

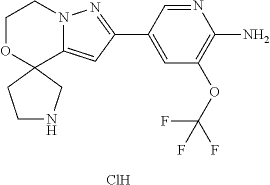 [SMILES: Nc1ncc(-c2cc3n(n2)CCOC32CCNC2)cc1OC(F)(F)F ; Cl]

(rac)-tert-butyl-2-[6-amino-5-(trifluoromethoxy)pyridin-3-yl]-6,7-dihydrospiro[pyrazolo[5,1-c][1,4]oxazine-4,3′-pyrrolidine]-1′-carboxylate (2.00 g, 4.39 mmol) was solubilised in 1,4-dioxane (39 mL) and HCl (16.5 mL, 4.0 M in 1,4-dioxane, 66 mmol) was added. The mixture was stirred overnight at 50° C. It was concentrated under reduced pressure to give 1.20 g (70% yield) of the title compound.

LC-MS (method 1): Rt=0.82 min; MS (ESIpos): m/z=356 [M+H]⁺

¹H-NMR (400 MHz, DMSO-d6) δ[ppm]: 1.064 (16.00), 1.281 (0.58), 3.564 (0.41), 4.155 (0.44), 4.162 (0.47), 6.822 (0.83), 8.339 (0.48), 8.344 (0.47).

### Intermediate 314

### (rac)-3-(difluoromethoxy)-5-[6,7-dihydrospiro[pyrazolo[5,1-c][1,4]oxazine-4,3′-pyrrolidin]-2-yl]pyridin-2-amine-hydrochloride salt

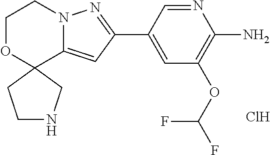 [SMILES: Nc1ncc(-c2cc3n(n2)CCOC32CCNC2)cc1OC(F)F ; Cl]

(rac)-tert-butyl-2-[6-amino-5-(difluoromethoxy)pyridin-3-yl]-6,7-dihydrospiro[pyrazolo[5,1-c][1,4]oxazine-4,3′-pyrrolidine]-1′-carboxylate (1.39 g, 3.18 mmol) was solubilised in 1,4-dioxane (28 mL) and HCl (11.9 mL, 4.0 M in 1,4-dioxane, 48 mmol) was added. The mixture was stirred overnight at 50° C. It was concentrated under reduced pressure to give 1.30 g of the title compound.

LC-MS (method 2): Rt=0.50 min; MS (ESIpos): m/z=338 [M+H]⁺

¹H-NMR (400 MHz, DMSO-d6) δ[ppm]: 1.063 (16.00), 2.518 (2.43), 2.523 (1.66), 3.314 (0.47), 3.383 (0.53), 4.157 (0.88), 4.164 (1.10), 4.178 (1.12), 6.875 (2.57), 7.221 (0.56), 7.402 (1.19), 7.582 (0.52), 8.009 (1.03), 8.207 (1.54), 8.211 (1.48).

### Intermediate 315

### 5-[(3′R)-6,7-dihydrospiro[pyrazolo[5,1-c][1,4]oxazine-4,3′-pyrrolidin]-2-yl]-3-(trifluoromethoxy)pyridin-2-amine hydrogen chloride

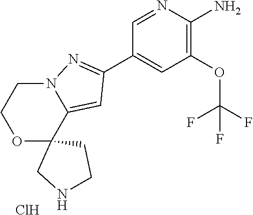 [SMILES: Nc1ncc(-c2cc3n(n2)CCO[C@@]32CCNC2)cc1OC(F)(F)F ; Cl]

To a mixture of tert-butyl (3′R)-2-[6-amino-5-(trifluoromethoxy)pyridin-3-yl]-6,7-dihydrospiro[pyrazolo[5,1-c][1,4]oxazine-4,3′-pyrrolidine]-1′-carboxylate (534 mg, 1.17 mmol) in Dioxane (5.8 mL), HCl (5.9 ml, 4.0 M in dioxane, 23 mmol) was added and the solution was stirred at RT overnight. The mixture was concentrated in vacuo to give 760 mg of the title compound.

LC-MS (Method 1): Rₜ=0.83 min; MS (ESIpos): m/z=356 [M+H]⁺

¹H-NMR (400 MHz, DMSO-d6) δ[ppm]: 1.064 (16.00), 1.154 (2.10), 2.518 (0.58), 4.148 (0.68), 4.154 (0.79), 4.161 (0.81), 6.818 (1.06), 8.338 (0.61), 8.343 (0.57).

### Intermediate 316

### 5-[(3R)-5′,6′-dihydrospiro[pyrrolidine-3,4′-pyrrolo[1,2-b]pyrazol]-2′-yl]-3-(trifluoromethyl)pyridin-2-amine hydrogen chloride

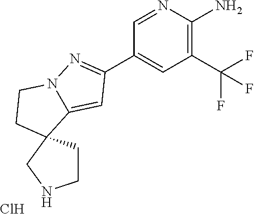 [SMILES: Nc1ncc(-c2cc3n(n2)CC[C@@]32CCNC2)cc1C(F)(F)F ; Cl]

To a mixture of tert-butyl (3R)-2′-[6-amino-5-(trifluoromethyl)pyridin-3-yl]-5′,6′-dihydrospiro[pyrrolidine-3,4′-pyrrolo[1,2-b]pyrazole]-1-carboxylate (2.90 g, 6.85 mmol) in Dioxane (34 mL), HCl (34 ml, 4.0 M in dioxane, 140 mmol) was added. The solution was stirred at RT overnight. The resultant mixture was concentrated in vacuo to give 3.10 g of the title compound.

LC-MS (Method 1): Rₜ=0.83 min; MS (ESIpos): m/z=324 [M+H]⁺

¹H-NMR (400 MHz, DMSO-d6) δ[ppm]: 1.064 (16.00), 1.122 (0.62), 1.154 (0.46), 2.144 (0.45), 2.156 (0.50), 2.163 (1.04), 2.171 (0.76), 2.183 (0.64), 2.188 (0.62), 2.522 (0.93), 2.580 (0.52), 2.594 (0.53), 2.678 (0.45), 2.696 (0.54), 2.712 (0.48), 3.287 (0.49), 3.402 (0.44), 3.409 (0.46), 3.419 (0.60), 3.436 (0.56), 3.448 (0.54), 3.564 (1.08), 4.184 (0.64), 4.192 (0.67), 4.203 (0.95), 4.211 (0.84), 4.219 (0.68), 4.226 (0.60), 6.666 (3.69), 8.107 (1.24), 8.112 (1.27), 8.584 (1.30), 8.589 (1.26).

### Intermediate 317

### 3-(difluoromethoxy)-5-[(3′R)-6,7-dihydrospiro[pyrazolo[5,1-c][1,4]oxazine-4,3′-pyrrolidin]-2-yl]pyridin-2-amine hydrogen chloride

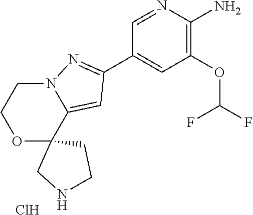 [SMILES: Nc1ncc(-c2cc3n(n2)CCO[C@@]32CCNC2)cc1OC(F)F ; Cl]

ICDE-245-1->to a mixture of tert-butyl (3′R)-2-[6-amino-5-(difluoromethoxy)pyridin-3-yl]-6,7-dihydrospiro[pyrazolo[5,1-c][1,4]oxazine-4,3′-pyrrolidine]-1′-carboxylate (382 mg, 873 μmol) in Dioxane, HCl (4 mol/L in Dioxane) was added->the solution was stirred at RT overnight, UPLC: ok->the mixture was concentrated in vacuo->the residue was stirred in Diethylether for 30 min., then filtered and dried-> to give 311 mg (95% yield) 311 mg as a beige solid in a ˜95% yield

LC-MS (Method 1): Rₜ=0.74 min; MS (ESIpos): m/z=338 [M+H]⁺

¹H-NMR (400 MHz, DMSO-d6) δ[ppm]: 1.064 (16.00), 1.069 (3.64), 1.087 (7.71), 1.104 (3.65), 1.399 (0.79), 1.424 (0.68), 2.518 (1.50), 2.523 (1.02), 3.288 (0.53), 3.305 (0.54), 3.319 (0.73), 3.336 (0.49), 3.352 (1.47), 3.370 (3.39), 3.384 (0.79), 3.387 (3.66), 3.405 (1.19), 3.698 (0.48), 4.105 (0.48), 4.119 (0.51), 4.155 (1.46), 4.162 (1.89), 4.174 (1.81), 6.852 (3.96), 7.188 (0.81), 7.369 (1.73), 7.550 (0.75), 7.949 (1.34), 8.205 (2.34), 8.210 (2.24).

### Intermediate 318

### 3-(difluoromethoxy)-5-[(3R)-5′,6′-dihydrospiro[pyrrolidine-3,4′-pyrrolo[1,2-b]pyrazol]-2′-yl]pyridin-2-amine hydrogen chloride

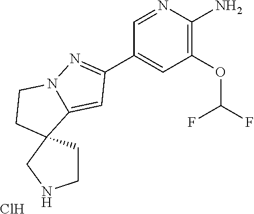 [SMILES: Nc1ncc(-c2cc3n(n2)CC[C@@]32CCNC2)cc1OC(F)F ; Cl]

To a mixture of tert-butyl (3R)-2′-[6-amino-5-(difluoromethoxy)pyridin-3-yl]-5′,6′-dihydrospiro[pyrrolidine-3,4′-pyrrolo[1,2-b]pyrazole]-1-carboxylate (368 mg, 873 μmol) in Dioxane (4.4 mL), HCl (4.4 ml, 4.0 M in dioxane, 17 mmol) was added. The solution was stirred at RT overnight. The mixture was concentrated and the residue was stirred in Diethylether for 30 min, then filtered and concentrated to give 340 mg of the title compound.

LC-MS (Method 1): Rₜ=0.76 min; MS (ESIpos): m/z=322 [M+H]⁺

¹H-NMR (400 MHz, DMSO-d6) δ[ppm]: 1.064 (16.00), 1.070 (1.51), 1.087 (2.15), 1.105 (1.09), 1.108 (0.63), 1.154 (0.71), 2.144 (0.52), 2.163 (1.14), 2.178 (0.74), 2.183 (0.66), 2.192 (0.61), 2.518 (2.11), 2.523 (1.47), 2.583 (0.54), 2.598 (0.56), 2.712 (0.56), 2.728 (0.50), 3.254 (0.47), 3.268 (0.42), 3.283 (0.64), 3.343 (0.42), 3.353 (0.67), 3.371 (1.27), 3.384 (0.98), 3.388 (1.19), 3.405 (0.69), 3.412 (0.80), 3.426 (0.76), 3.442 (0.78), 3.455 (0.47), 4.198 (0.72), 4.207 (0.80), 4.213 (1.03), 4.222 (0.92), 4.227 (0.96), 4.232 (0.78), 4.241 (0.68), 6.681 (3.85), 7.189 (0.77), 7.370 (1.54), 7.551 (0.70), 7.955 (1.31), 8.178 (2.13), 8.182 (2.10), 9.688 (0.61).

### Intermediate 319

### 5-[(3′R)-6,7-dihydrospiro[pyrazolo[5,1-c][1,4]oxazine-4,3′-pyrrolidin]-2-yl]-3-(trifluoromethyl)pyridin-2-amine hydrogen chloride

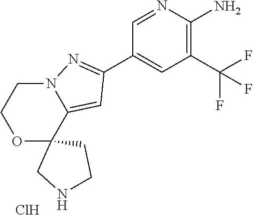 [SMILES: Nc1ncc(-c2cc3n(n2)CCO[C@@]32CCNC2)cc1C(F)(F)F ; Cl]

To a mixture of tert-butyl (3′R)-2-[6-amino-5-(trifluoromethyl)pyridin-3-yl]-6,7-dihydrospiro[pyrazolo[5,1-c][1,4]oxazine-4,3′-pyrrolidine]-1′-carboxylate (448 mg, 1.02 mmol) in Dioxane (5 mL), HCl (5.1 ml, 4.0 M in dioxane, 20 mmol) was added. The solution was stirred at RT overnight. The mixture was concentrated to give 480 mg of the title compound.

LC-MS (Method 1): Rₜ=0.81 min; MS (ESIpos): m/z=341 [M+H]⁺

¹H-NMR (400 MHz, DMSO-d6) δ[ppm]: 2.238 (0.58), 2.262 (1.07), 2.272 (1.06), 2.289 (0.90), 2.299 (1.39), 2.323 (2.56), 2.327 (2.83), 2.331 (1.88), 2.336 (0.83), 2.518 (10.46), 2.523 (7.21), 2.660 (0.80), 2.665 (1.78), 2.669 (2.52), 2.673 (1.73), 2.678 (0.76), 3.280 (0.71), 3.294 (1.80), 3.312 (1.91), 3.325 (2.35), 3.343 (1.51), 3.359 (0.83), 3.384 (2.87), 3.457 (0.47), 3.469 (0.82), 3.478 (0.95), 3.486 (1.55), 3.488 (1.51), 3.496 (1.44), 3.498 (1.43), 3.516 (1.08), 3.547 (0.51), 3.587 (0.66), 3.598 (0.53), 3.605 (0.40), 3.662 (0.55), 3.666 (0.64), 3.685 (1.17), 3.699 (1.50), 3.700 (1.51), 3.713 (1.20), 3.731 (1.04), 3.818 (0.79), 3.827 (0.51), 3.829 (0.91), 3.832 (0.49), 3.841 (1.07), 4.018 (1.26), 4.102 (0.45), 4.114 (1.23), 4.122 (2.19), 4.131 (1.27), 4.161 (9.94), 4.181 (1.52), 4.203 (0.55), 4.276 (0.53), 4.313 (0.90), 4.319 (0.79), 4.329 (3.90), 4.411 (2.64), 4.420 (2.35), 4.423 (2.84), 4.426 (2.49), 4.428 (2.39), 4.434 (3.13), 4.499 (4.03), 4.636 (1.25), 6.071 (0.51), 6.342 (1.37), 6.347 (1.56), 6.836 (16.00), 7.479 (1.31), 7.485 (1.16), 8.096 (4.36), 8.101 (4.41), 8.136 (2.26), 8.598 (4.41), 8.601 (4.34), 9.441 (0.80), 9.863 (0.82).

### Intermediate 320

### 5-[(3'S)-6,7-dihydrospiro[pyrazolo[5,1-c][1,4]oxazine-4,3′-pyrrolidin]-2-yl]-3-(trifluoromethyl)pyridin-2-amine-hydrochloride salt

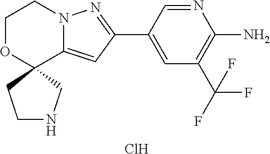 [SMILES: Nc1ncc(-c2cc3n(n2)CCO[C@]32CCNC2)cc1C(F)(F)F ; Cl]

Intermediate 320 was prepared in analogy to Intermediate 319 using tert-butyl-(3'S)2-[6-amino-5-(trifluoromethyl)pyridin-3-yl]-6,7-dihydrospiro[pyrazolo[5,1-c][1,4]oxazine-4,3′-pyrrolidine]-1′-carboxylate (203 mg, 462 μmol).

LC-MS (method 2): Rt=0.62 min; MS (ESIpos): m/z=340 [M+H]⁺

¹H-NMR (400 MHz, DMSO-d6) δ[ppm]: 1.154 (1.60), 1.230 (0.58), 2.233 (0.53), 2.257 (1.07), 2.267 (0.98), 2.284 (0.88), 2.294 (1.35), 2.318 (1.11), 2.322 (1.06), 2.327 (1.14), 2.332 (0.78), 2.463 (1.34), 2.518 (3.84), 2.523 (2.52), 2.665 (0.76), 2.669 (1.08), 2.673 (0.76), 3.273 (0.61), 3.292 (1.63), 3.309 (1.53), 3.323 (2.29), 3.341 (1.35), 3.354 (0.75), 3.366 (0.47), 3.384 (0.59), 3.469 (0.50), 3.485 (0.81), 3.487 (0.78), 3.498 (1.09), 3.514 (0.93), 3.681 (0.98), 3.698 (1.27), 3.713 (1.11), 3.728 (0.90), 4.158 (8.69), 4.162 (9.17), 5.418 (1.21), 6.851 (16.00), 8.134 (4.43), 8.139 (4.37), 8.604 (4.60), 8.607 (4.38), 9.486 (0.79), 9.977 (0.81).

### Intermediate 321

### 2-amino-5-[(3'S)-6,7-dihydrospiro[pyrazolo[5,1-c][1,4]oxazine-4,3′-pyrrolidin]-2-yl]nicotinonitrile-hydrochloride salt

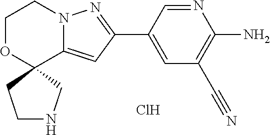 [SMILES: N#Cc1cc(-c2cc3n(n2)CCO[C@]32CCNC2)cnc1N ; Cl]

Intermediate 321 was prepared in analogy to Intermediate 319 using tert-butyl-(3'S)-2-(6-amino-5-cyanopyridin-3-yl)-6,7-dihydrospiro[pyrazolo[5,1-c][1,4]oxazine-4,3′-pyrrolidine]-1′-carboxylate (320 mg, 807 μmol).

[α]²⁰_{D}: −17.3° (c=1.00, DMSO)

LC-MS (method 1): Rt=0.66 min; MS (ESIpos): m/z=297 [M+H]⁺

### Intermediate 322

### 2-amino-5-[(3′R)-6,7-dihydrospiro[pyrazolo[5,1-c][1,4]oxazine-4,3′-pyrrolidin]-2-yl]nicotinonitrile hydrochloride salt

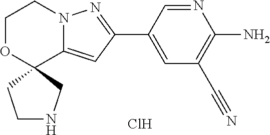 [SMILES: N#Cc1cc(-c2cc3n(n2)CCO[C@@]32CCNC2)cnc1N ; Cl]

Intermediate 322 was prepared in analogy to Intermediate 319 using tert-butyl-2-(6-amino-5-cyanopyridin-3-yl)-6,7-dihydrospiro[pyrazolo[5,1-c][1,4]oxazine-4,3′-pyrrolidine]-1′-carboxylate (450 mg, 1.14 mmol, stereoisomer 2).

[α]²⁰_{D}: +6.2° (c=1.00, DMSO)

LC-MS (method 1): Rₜ=0.66 min; MS (ESIpos): m/z=297 [M+H]⁺

¹H-NMR (400 MHz, DMSO-d6) δ[ppm]: 1.064 (16.00), 2.518 (1.23), 2.523 (0.81), 3.299 (0.42), 4.152 (3.52), 4.872 (0.56), 6.771 (3.16), 8.177 (1.60), 8.183 (1.56), 8.602 (2.01), 8.607 (2.06).

### Intermediate 323

### (rac)-methyl 2-amino-5-[1-(ethylcarbamoyl)-5′,6′-dihydrospiro[pyrrolidine-3,4′-pyrrolo[1,2-b]pyrazol]-2′-yl]pyridine-3-carboxylate

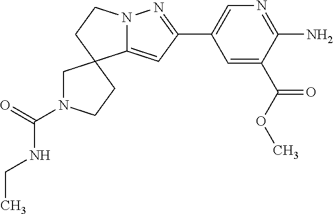 [SMILES: CCNC(=O)N1CCC2(CCn3nc(-c4cnc(N)c(C(=O)OC)c4)cc32)C1]

(rac)-1-(ethylcarbamoyl)-5′,6′-dihydrospiro[pyrrolidine-3,4′-pyrrolo[1,2-b]pyrazol]-2′-yl trifluoromethanesulfonate (917 mg, 2.40 mmol) was solubilised in 1,4-dioxane (20 mL) under nitrogen, methyl 2-amino-5-(4,4,5,5-tetramethyl-1,3,2-dioxaborolan-2-yl)pyridine-3-carboxylate (1.00 g, 3.60 mmol), K3PO4 (14 mL, 0.50 M, 7.2 mmol) and XPhos Pd G2 (94.3 mg, 120 μmol) were added and the mixture was stirred overnight at 100° C. The mixture was diluted with ethyl acetate and washed with brine. The organic layer was dried over s silicone filter and evaporated. The residue was purified by flash chromatography to give 510 mg (55% yield) of the title compound.

LC-MS (method 1): Rt=0.82 min; MS (ESIpos): m/z=385 [M+H]⁺

1H-NMR (400 MHz, DMSO-d6) δ[ppm]: 0.990 (0.17), 1.003 (0.60), 1.021 (1.11), 1.035 (0.42), 1.038 (0.49), 1.053 (0.66), 1.066 (16.00), 1.070 (0.88), 1.156 (2.03), 2.066 (0.17), 2.323 (0.29), 2.327 (0.40), 2.332 (0.28), 2.518 (1.15), 2.523 (0.98), 2.541 (0.21), 2.665 (0.26), 2.669 (0.37), 2.673 (0.27), 3.038 (0.26), 3.052 (0.24), 3.055 (0.24), 3.069 (0.18), 3.404 (0.63), 3.844 (1.90), 3.938 (1.36), 4.162 (0.17), 4.179 (0.25), 6.415 (0.67), 7.241 (0.31), 8.383 (0.34), 8.389 (0.34), 8.609 (0.39), 8.615 (0.37).

### Intermediate 324

### (rac)-1-(1,4-dimethyl-1H-pyrazol-5-yl)ethan-1-ol

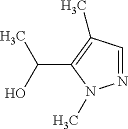

1-(1,4-Dimethyl-1H-pyrazol-5-yl)ethan-1-one (300 mg, 2.17 mmol) was solubilized in methanol (7.5 mL) and the reaction was cooled to 0° C. Sodium borohydride (32.9 mg, 868 μmol) was added and the mixture was stirred at rt for 1 h. The reaction mixture was diluted with dichloromethane, washed with water and sat. sodium chloride solution, dried over sodium sulfate, filtered and concentrated under reduced pressure to give 193 mg (95% purity, 60% yield) of the title compound that was used without further purification in the next step.

LC-MS (Method 1): Rₜ=0.59 min; MS (ESIpos): m/z=141 [M+H]⁺

¹H-NMR (400 MHz, CHLOROFORM-d): δ [ppm]=1.64 (d, 3H), 2.16 (s, 3H), 2.26 (d, 1H), 4.00 (s, 3H), 5.18 (dd, 1H), 7.36 (s, 1H).

SnAr NO2

### Intermediate 325

### (rac)-5-bromo-3-{[1-(6-methylpyridin-2-yl)ethyl]oxy}-2-nitropyridine

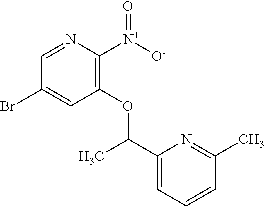 [SMILES: Cc1cccc(C(C)Oc2cc(Br)cnc2[N+](=O)[O-])n1]

(Rac)-1-(6-Methylpyridin-2-yl)ethan-1-ol (200 mg, 1.46 mmol) was solubilised in THF (7.0 mL) and the reaction mixture was cooled to 0° C. Sodium hydride (79.5 mg, 60% purity, 1.99 mmol) was then slowly added and the reaction mixture was stirred at 0° C. for 30 min. A solution of 5-bromo-3-fluoro-2-nitropyridine (293 mg, 1.33 mmol) in THF (7.0 mL) was added slowly at 0° C. and the reaction mixture was stirred for 1 h at 0° C. The reaction mixture was then slowly quenched with water and the organic solvent was removed under reduced pressure. The aqueous phase was extracted with dichloromethane and the organic phase was dried over sodium sulfate, filtered and concentrated under reduced pressure to give 440 mg (65% purity, 63% yield) of the title compound which was used without further purification in the next step.

LC-MS (Method 1): Rₜ=1.25 min; MS (ESIpos): m/z=338 [M+H]⁺

### Intermediate 326

### (rac)-5-bromo-3-{[1-(5-methylpyridin-2-yl)ethyl]oxy}-2-nitropyridine

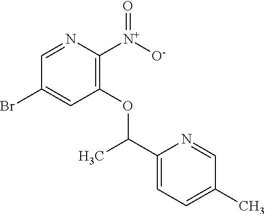 [SMILES: Cc1ccc(C(C)Oc2cc(Br)cnc2[N+](=O)[O-])nc1]

The compound was prepared similarly to Intermediate 325 starting from (rac)-1-(5-methylpyridin-2-yl)ethan-1-ol (215 mg, 1.57 mmol) to give 490 mg (70% purity, 71% yield) of the title compound.

LC-MS (Method 1): Rₜ=1.24 min; MS (ESIpos): m/z=338 [M+H]⁺

### Intermediate 327

### (rac)-5-bromo-3-{[1-(4-methylpyridin-2-yl)ethyl]oxy}-2-nitropyridine

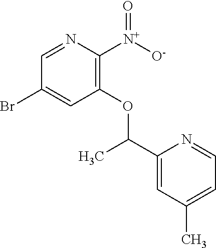 [SMILES: Cc1ccnc(C(C)Oc2cc(Br)cnc2[N+](=O)[O-])c1]

The compound was prepared similarly to Intermediate 325 starting from (rac)-1-(4-methylpyridin-2-yl)ethan-1-ol (206 mg, 1.50 mmol) to give 466 mg (57% purity, 57% yield) of the title compound.

LC-MS (Method 1): Rₜ=1.23 min; MS (ESIpos): m/z=338 [M+H]⁺

### Intermediate 328

### (rac)-5-bromo-3-{[1-(3-methylpyridin-2-yl)ethyl]oxy}-2-nitropyridine

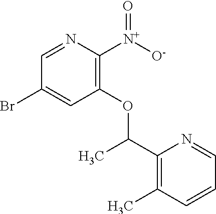 [SMILES: Cc1cccnc1C(C)Oc1cc(Br)cnc1[N+](=O)[O-]]

The compound was prepared similarly to Intermediate 325 starting from (rac)-1-(3-methylpyridin-2-yl)ethan-1-ol (90.0 mg, 656 μmol) to give 240 mg (74% purity, 88% yield) of the title compound.

LC-MS (Method 1): Rₜ=1.21 min; MS (ESIpos): m/z=338 [M+H]⁺

### Intermediate 329

### (rac)-5-bromo-3-[1-(1,4-dimethyl-1H-pyrazol-5-yl)ethoxy]-2-nitropyridine

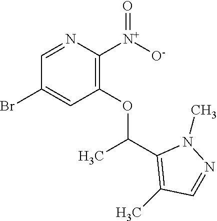 [SMILES: Cc1cnn(C)c1C(C)Oc1cc(Br)cnc1[N+](=O)[O-]]

The compound was prepared similarly to Intermediate 325 starting from (rac)-1-(1,4-dimethyl-1H-pyrazol-5-yl)ethan-1-ol (190 mg, 1.36 mmol) to give 452.7 mg (60% purity, 58% yield) of the title compound.

LC-MS (Method 1): Rₜ=1.13 min; MS (ESIpos): m/z=343 [M+H]⁺

### Intermediate 330

### 5-bromo-3-[(1S)-1-(2,6-difluorophenyl)ethoxy]-2-nitropyridine

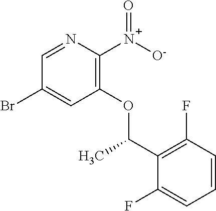 [SMILES: C[C@H](Oc1cc(Br)cnc1[N+](=O)[O-])c1c(F)cccc1F]

(1S)-1-(2,6-Difluorophenyl)ethan-1-ol (150 mg, 948 μmol) was dissolved in THF (4 mL) and cooled to 0° C. Sodium hydride (41.7 mg, 60% purity, 1.04 mmol) was added slowly and the mixture was stirred for 30 min at 0° C. A solution of 5-bromo-3-fluoro-2-nitropyridine (210 mg, 948 μmol) in THF (4 mL) was added slowly and the mixture was stirred for 1 h at 0° C. The reaction mixture was quenched with water and extracted with ethyl acetate. The combined organic layers were dried over sodium sulfate, filtered and concentrated to give 364 mg (80% purity, 85% yield) of the title compound which was used without further purification in the next step.

### Intermediate 331

### 5-bromo-3-[(1R)-1-(5-fluoropyridin-2-yl)ethoxy]-2-nitropyridine

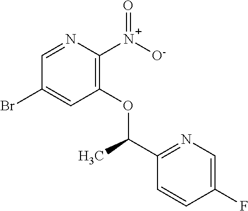 [SMILES: C[C@@H](Oc1cc(Br)cnc1[N+](=O)[O-])c1ccc(F)cn1]

The compound was prepared similarly to Intermediate 330 starting from (1R)-1-(5-fluoropyridin-2-yl)ethan-1-ol (150 mg, 1.06 mmol) to give 370 mg (75% purity, 76% yield) of the title compound.

LC-MS (Method 1): Rₜ=1.23 min; MS (ESIpos): m/z=342 [M+H]⁺

### Intermediate 332

### 5-bromo-3-[(1R)-1-(3-fluoropyridin-2-yl)ethoxy]-2-nitropyridine

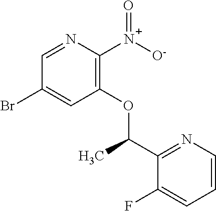 [SMILES: C[C@@H](Oc1cc(Br)cnc1[N+](=O)[O-])c1ncccc1F]

The compound was prepared similarly to Intermediate 330 starting from (1R)-1-(3-fluoropyridin-2-yl)ethan-1-ol (150 mg, 1.06 mmol) to give 370 mg (65% purity, 66% yield) of the title compound.

LC-MS (Method 1): Rₜ=1.18 min; MS (ESIpos): m/z=342 [M+H]⁺

### Intermediate 333

### 5-bromo-3-[(1R)-1-(2,6-difluorophenyl)ethoxy]-2-nitropyridine

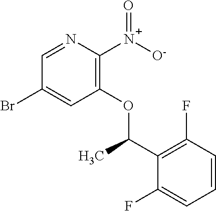 [SMILES: C[C@@H](Oc1cc(Br)cnc1[N+](=O)[O-])c1c(F)cccc1F]

The compound was prepared similarly to Intermediate 330 starting from (1R)-1-(2,6-difluorophenyl)ethan-1-ol (150 mg, 948 μmol) to give 322 mg (80% purity, 76% yield) of the title compound.

LC-MS (Method 1): Rₜ=1.35 min; MS (ESIpos): m/z=359 [M+H]⁺

¹H NMR (400 MHz, CHLOROFORM-d, 22° C.) δ ppm 1.85 (d, 3H), 5.82 (q, 1H), 6.93 (t, 2H), 7.31 (tt, 1H), 7.58 (d, 1H), 8.08 (d, 1H).

### Intermediate 334

### (rac)-2-{1-[(5-bromo-2-nitropyridin-3-yl)oxy]ethyl}benzonitrile

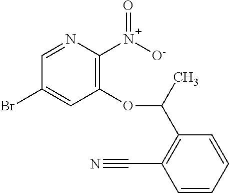 [SMILES: CC(Oc1cc(Br)cnc1[N+](=O)[O-])c1ccccc1C#N]

THF (6 mL) was initially introduced and cooled to 0° C. Then, (rac)-2-[1-hydroxyethyl]benzonitrile (140 mg, 950 μmol) and sodium hydride (45.2 mg, 60% purity, 1.13 mmol) were added and the mixture was stirred for 30 min at 0° C. A solution of 5-bromo-3-fluoro-2-nitropyridine (200 mg, 905 μmol) in THF (4 mL) was added dropwise and the mixture was stirred for 2 h at 0° C. The reaction mixture was quenched with water and extracted with ethyl acetate. The combined organic layers were dried over a hydrophobic filter and concentrated to give 333 mg (83% purity, 88% yield) of the title compound.

LC-MS (Method 1): Rₜ=1.25 min; MS (ESIpos): m/z=348 [M+H]⁺

¹H-NMR (400 MHz, DMSO-d6) δ[ppm]: 0.806 (0.40), 0.822 (0.51), 0.831 (0.60), 0.850 (0.73), 1.171 (0.41), 1.232 (1.95), 1.352 (2.14), 1.485 (0.44), 1.502 (0.43), 1.552 (4.42), 1.568 (4.58), 1.683 (15.91), 1.699 (16.00), 1.839 (0.43), 2.523 (1.96), 5.698 (0.66), 5.715 (0.63), 6.107 (1.02), 6.124 (3.35), 6.140 (3.33), 6.155 (0.97), 7.542 (2.05), 7.545 (2.13), 7.561 (4.29), 7.563 (4.34), 7.580 (2.97), 7.582 (2.75), 7.597 (1.05), 7.616 (0.67), 7.668 (3.09), 7.687 (5.07), 7.705 (1.05), 7.706 (1.02), 7.759 (2.63), 7.762 (2.83), 7.770 (0.92), 7.773 (1.12), 7.778 (3.64), 7.781 (3.71), 7.791 (1.09), 7.797 (1.57), 7.801 (1.57), 7.807 (0.47), 7.822 (0.89), 7.841 (0.76), 7.900 (3.88), 7.903 (3.82), 7.920 (3.53), 7.922 (3.34), 8.066 (0.40), 8.303 (6.06), 8.307 (10.67), 8.317 (9.08), 8.321 (5.28).

### Intermediate 335

### 3-{(1R)-1-[(5-bromo-2-nitropyridin-3-yl)oxy]ethyl}benzonitrile

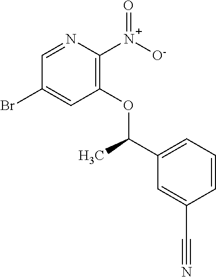 [SMILES: C[C@@H](Oc1cc(Br)cnc1[N+](=O)[O-])c1cccc(C#N)c1]

3-[(1R)-1-Hydroxyethyl]benzonitrile (150 mg, 1.02 mmol) was dissolved in THF (5 mL) and cooled to 0° C. Sodium hydride (44.8 mg, 60% purity, 1.12 mmol) was added slowly and the reaction mixture was stirred for 30 min at 0° C. A solution of 5-bromo-3-fluoro-2-nitropyridine 225 mg, 1.02 mmol) in THF (4 mL) was added slowly and the mixture was stirred for 1 h at 0° C. The reaction mixture was quenched with water and extracted with ethyl acetate. The combined organic layers were dried over sodium sulfate, filtered and concentrated to give 371 mg (50% purity, 52% yield) of the title compound.

LC-MS (Method 1): Rₜ=1.25 min; MS (ESIpos): m/z=365 [M+NH₄]⁺

### Intermediate 336

### 4-{(1R)-1-[(5-bromo-2-nitropyridin-3-yl)oxy]ethyl}benzonitrile

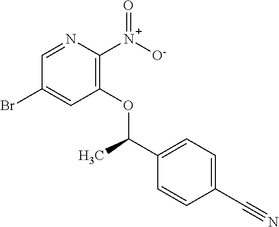 [SMILES: C[C@@H](Oc1cc(Br)cnc1[N+](=O)[O-])c1ccc(C#N)cc1]

The compound was prepared similarly to Intermediate 335 starting from 4-[(1R)-1-hydroxyethyl]benzonitrile (150 mg, 1.02 mmol) to give 363 mg (45% purity, 46% yield) of the title compound.

LC-MS (Method 1): Rₜ=1.26 min; MS (ESIpos): m/z=365 [M+NH₄]+

### Intermediate 337

### 5-bromo-3-[(1R)-1-(3,5-difluoropyridin-4-yl)ethoxy]-2-nitropyridine

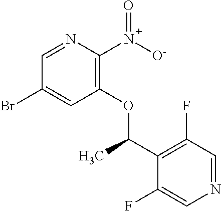 [SMILES: C[C@@H](Oc1cc(Br)cnc1[N+](=O)[O-])c1c(F)cncc1F]

The compound was prepared similarly to Intermediate 335 starting from (1R)-1-(3,5-difluoropyridin-4-yl)ethan-1-ol (150 mg, 943 μmol) to give 332 mg (60% purity, 59% yield) of the title compound.

LC-MS (Method 1): Rₜ=1.17 min; MS (ESIpos): m/z=360 [M+H]⁺

### Intermediate 338

### (rac)-5-bromo-2-nitro-3-(1-phenylpropoxy)pyridine

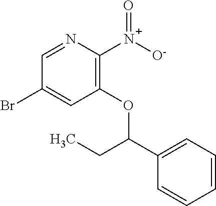 [SMILES: CCC(Oc1cc(Br)cnc1[N+](=O)[O-])c1ccccc1]

The compound was prepared similarly to Intermediate 335 starting from (rac)-1-phenylpropan-1-ol (240 μL, 67% purity, 1.2 mmol) to give 324 mg (75% purity, 64% yield) of the title compound which was used without further purification in the next step.

LC-MS (Method 1): Rₜ=1.44 min; MS (ESIneg): m/z=335 [M−H]⁻

### Intermediate 339

### 3-{(1S)-1-[(5-bromo-2-nitropyridin-3-yl)oxy]ethyl}benzonitrile

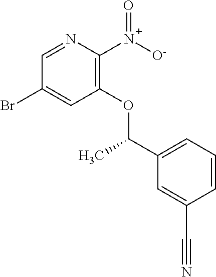 [SMILES: C[C@H](Oc1cc(Br)cnc1[N+](=O)[O-])c1cccc(C#N)c1]

The compound was prepared similarly to Intermediate 335 starting from 3-[(1S)-1-hydroxyethyl]benzonitrile (150 mg, 1.02 mmol) to give 374 mg (55% purity, 58% yield) of the title compound.

LC-MS (Method 1): Rₜ=1.25 min; MS (ESIpos): m/z=365 [M+NH₄]+

### Intermediate 340

### 4-{(1S)-1-[(5-bromo-2-nitropyridin-3-yl)oxy]ethyl}benzonitrile

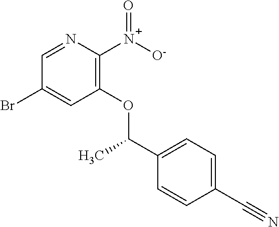 [SMILES: C[C@H](Oc1cc(Br)cnc1[N+](=O)[O-])c1ccc(C#N)cc1]

The compound was prepared similarly to Intermediate 335 starting from 4-[(1S)-1-hydroxyethyl]benzonitrile (150 mg, 1.02 mmol) to give 366 mg (50% purity, 52% yield) of the title compound.

LC-MS (Method 1): Rₜ=1.26 min; MS (ESIpos): m/z=348 [M+N]+

### Intermediate 341

### 5-bromo-3-[(1S)-1-(3,5-difluoropyridin-4-yl)ethoxy]-2-nitropyridine

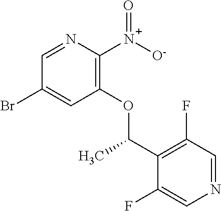 [SMILES: C[C@H](Oc1cc(Br)cnc1[N+](=O)[O-])c1c(F)cncc1F]

The compound was prepared similarly to Intermediate 335 starting from (1S)-1-(3,5-difluoropyridin-4-yl)ethan-1-ol (162 mg, 1.02 mmol) to give 337 mg (68% purity, 62% yield) of the title compound.

LC-MS (Method 1): Rₜ=1.18 min; MS (ESIpos): m/z=360 [M+H]⁺

### Intermediate 342

### 5-bromo-2-nitro-3-[(1R)-1-phenylethoxy]pyridine

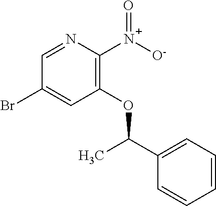 [SMILES: C[C@@H](Oc1cc(Br)cnc1[N+](=O)[O-])c1ccccc1]

The compound was prepared similarly to Intermediate 335 starting from (1R)-1-phenylethan-1-ol (230 μL, 63% purity, 1.2 mmol) to give 349 mg (81% purity, 77% yield) of the title compound.

LC-MS (Method 1): Rₜ=1.36 min; MS (ESIpos): m/z=323 [M+H]⁺

### Intermediate 343

### (rac)-5-bromo-3-[1-(1-methyl-1H-pyrazol-5-yl)ethoxy]-2-nitropyridine

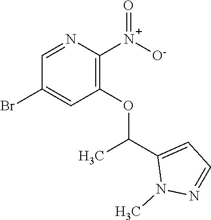 [SMILES: CC(Oc1cc(Br)cnc1[N+](=O)[O-])c1ccnn1C]

The compound was prepared similarly to Intermediate 335 starting from 1-(1-methyl-1H-pyrazol-5-yl)ethan-1-ol (200 mg, 1.59 mmol) to give 379 mg (98% purity, 72% yield) of the title compound.

LC-MS (Method 1): Rₜ=1.06 min; MS (ESIpos): m/z=327 [M+H]⁺

¹H-NMR (400 MHz, CHLOROFORM-d) 5 [ppm]: 1.791 (6.34), 1.807 (6.55), 3.946 (16.00), 5.595 (1.14), 5.612 (1.13), 6.324 (2.12), 6.328 (2.15), 7.459 (1.97), 7.463 (1.97), 7.546 (2.11), 7.550 (2.13), 8.151 (2.49), 8.157 (2.40).

### Intermediate 344

### 5-bromo-3-[(1R)-1-(2-fluorophenyl)ethoxy]-2-nitropyridine

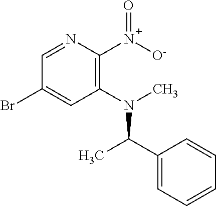 [SMILES: C[C@H](c1ccccc1)N(C)c1cc(Br)cnc1[N+](=O)[O-]]

The compound was prepared similarly to Intermediate 335 starting from (1R)-1-(2-fluorophenyl)ethan-1-ol (250 mg, 1.78 mmol) to give 495 mg (90% purity, 73% yield) of the title compound.

LC-MS (Method 1): Rₜ=1.36 min; MS (ESIpos): m/z=341 [M+H]⁺

¹H-NMR (400 MHz, CHLOROFORM-d) 5 [ppm]: 1.682 (1.18), 1.698 (0.95), 1.708 (0.56), 1.718 (16.00), 1.734 (15.84), 5.776 (0.82), 5.792 (2.57), 5.807 (2.56), 5.823 (0.80), 7.092 (1.44), 7.095 (1.48), 7.112 (1.79), 7.116 (2.02), 7.117 (1.70), 7.121 (1.68), 7.138 (1.76), 7.141 (1.81), 7.173 (1.26), 7.175 (1.24), 7.192 (2.91), 7.194 (2.71), 7.211 (1.87), 7.213 (1.69), 7.307 (1.01), 7.312 (1.13), 7.321 (1.02), 7.326 (1.87), 7.328 (1.14), 7.330 (1.05), 7.333 (1.12), 7.340 (1.02), 7.341 (1.11), 7.344 (1.15), 7.346 (1.52), 7.351 (0.76), 7.360 (0.68), 7.364 (0.65), 7.446 (1.32), 7.450 (1.26), 7.465 (2.37), 7.469 (2.22), 7.484 (1.33), 7.489 (1.24), 7.502 (5.59), 7.507 (5.64), 7.921 (0.45), 7.927 (0.42), 8.082 (7.50), 8.087 (7.61).

### Intermediate 345

### 5-bromo-N-methyl-2-nitro-N-[(1R)-1-phenylethyl]pyridin-3-amine

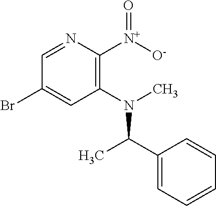 [SMILES: C[C@H](c1ccccc1)N(C)c1cc(Br)cnc1[N+](=O)[O-]]

5-Bromo-3-fluoro-2-nitropyridine (200 mg, 905 μmol), (1R)—N-methyl-1-phenylethan-1-amine (150 μL, 1000 μmol) and triethylamine (130 μL, 950 μmol) were dissolved in THF (4.0 mL) and stirred over night at rt. The mixture was stirred for 2 h at 60° C. and concentrated. The residue was purified by flash column chromatography (silica gel, hexane/ethyla acetate (0-20%) gradient) to give 184 mg (95% purity, 57% yield) of the title compound.

LC-MS (Method 1): Rₜ=1.42 min; MS (ESIpos): m/z=336 [M+H]⁺

¹H-NMR (400 MHz, CHLOROFORM-d) 5 [ppm]: 1.612 (7.58), 1.629 (7.65), 2.582 (16.00), 4.730 (0.93), 4.748 (0.91), 7.230 (1.72), 7.247 (2.24), 7.249 (2.19), 7.252 (1.78), 7.260 (14.60), 7.307 (1.31), 7.322 (0.65), 7.324 (1.01), 7.328 (0.48), 7.357 (2.25), 7.361 (0.87), 7.372 (1.44), 7.376 (2.66), 7.380 (0.53), 7.393 (0.95), 7.396 (0.52), 7.570 (2.55), 7.575 (2.62), 7.978 (3.05), 7.982 (3.01), 7.986 (0.42).

### Intermediate 346

### 5-bromo-3-[(1R)-1-(4-methylpyridin-2-yl)ethoxy]-2-nitropyridine

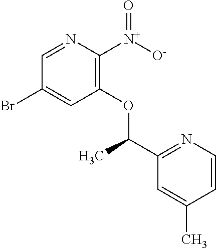 [SMILES: Cc1ccnc([C@@H](C)Oc2cc(Br)cnc2[N+](=O)[O-])c1]

The compound was prepared similarly to Intermediate 335 starting from (1R)-1-(4-methylpyridin-2-yl)ethan-1-ol (155 mg, 1.13 mmol) to give 290 mg (97% purity, 74% yield) of the title compound.

LC-MS (Method 1): Rₜ=1.23 min; MS (ESIpos): m/z=338 [M+H]⁺

¹H-NMR (400 MHz, CHLOROFORM-d) 5 [ppm]: 1.240 (0.48), 1.258 (0.86), 1.276 (0.42), 1.718 (9.75), 1.734 (10.33), 2.045 (1.67), 2.357 (13.58), 5.462 (0.55), 5.477 (1.85), 5.494 (1.85), 5.510 (0.56), 7.067 (0.97), 7.069 (1.21), 7.071 (1.23), 7.082 (1.26), 7.084 (1.26), 7.260 (16.00), 7.272 (2.20), 7.274 (2.40), 7.618 (3.29), 7.622 (3.43), 8.060 (4.12), 8.065 (4.31), 8.426 (2.05), 8.439 (2.04).

### Intermediate 347

### 5-bromo-3-[(1R)-1-(6-methylpyridin-2-yl)ethoxy]-2-nitropyridine

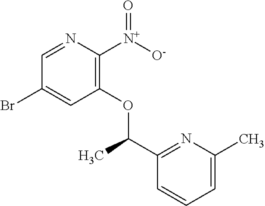 [SMILES: Cc1cccc([C@@H](C)Oc2cc(Br)cnc2[N+](=O)[O-])n1]

(1R)-1-(6-Methylpyridin-2-yl)ethan-1-ol (155 mg, 1.13 mmol) was dissolved in THF (6 mL) and cooled to 0° C. Sodium hydride (49.8 mg, 60% purity, 1.24 mmol) was added and the mixture was stirred for 30 min at 0° C. A solution of 5-bromo-3-fluoro-2-nitropyridine (250 mg, 1.13 mmol) in THF (4 mL) was added slowly at 0° C. and the mixture was stirred for 1 h at 0° C. and over night at rt. The reaction mixture was stirred for 6 h at 70° C. Sodium hydride (50 mg, 60% purity, 1.24 mmol) was added at rt and stirred over night at rt. Sodium hydride (100 mg, 60% purity, 2.48 mmol) was added again and the mixture was stirred over night at rt. The reaction mixture was diluted with water and extracted 2 times with ethyl acetate. The combined organic layers were dried over sodium sulfate, filtered and concentrated. The residue was purified by flash column chromatography (silica gel, hexane/ethyla acetate (0-45%) gradient) to give 158 mg (95% purity, 39% yield) of the title compound.

LC-MS (Method 1): Rₜ=1.26 min; MS (ESIpos): m/z=338 [M+H]⁺

¹H-NMR (400 MHz, CHLOROFORM-d) 5 [ppm]: 1.268 (0.75), 1.407 (0.43), 1.524 (3.09), 1.732 (11.25), 1.749 (10.85), 2.055 (1.11), 2.560 (0.80), 2.591 (16.00), 5.465 (0.55), 5.480 (1.77), 5.497 (1.79), 5.513 (0.56), 7.111 (1.78), 7.130 (2.00), 7.263 (1.71), 7.282 (2.08), 7.602 (1.68), 7.622 (3.10), 7.641 (1.40), 7.656 (3.39), 7.660 (3.55), 8.064 (5.31), 8.069 (5.30).

### Intermediate 348

### 5-bromo-3-[(1R)-1-(3-chlorophenyl)ethoxy]-2-nitropyridine

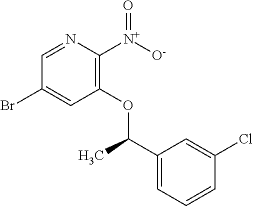 [SMILES: C[C@@H](Oc1cc(Br)cnc1[N+](=O)[O-])c1cccc(Cl)c1]

(1R)-1-(3-Chlorophenyl)ethan-1-ol (464 mg, 2.96 mmol) was dissolved in THF (5 mL), cooled to 0° C., sodium hydride (158 mg, 60% purity, 3.95 mmol) was added and the mixture was stirred for 30 min at 0° C. 5-Bromo-3-fluoro-2-nitropyridine (450 mg, 97% purity, 1.98 mmol) was dissolved in THF (5 mL) and added dropwise at 0° C. The mixture was stirred for 1 h at 0° C. and 30 min at room temperature. The reaction mixture was poured into water. THF was distilled off and the aqueous phase was extracted with dichloromethane. The organic phase was dried over a hydrophobic filter and concentrated. The residue was purified by column chromatography (silica gel, hexane/ethyl acetate (2-20%) gradient) to give 273 mg (98% purity, 38% yield) of the title compound.

[α]²⁰_{D}: +109.5° (c=1.00, methanol)

LC-MS (Method 2): Rₜ=1.44 min; MS (ESIpos): m/z=357 [M+H]⁺

¹H-NMR (400 MHz, DMSO-d6) δ[ppm]: 1.153 (0.96), 1.165 (0.66), 1.170 (1.94), 1.188 (0.91), 1.566 (16.00), 1.582 (15.88), 1.986 (3.61), 2.518 (1.01), 2.523 (0.67), 4.016 (0.80), 4.034 (0.80), 5.948 (1.01), 5.963 (3.44), 5.979 (3.40), 5.995 (0.96), 7.372 (1.93), 7.376 (2.90), 7.380 (3.85), 7.385 (2.98), 7.394 (6.03), 7.396 (9.16), 7.402 (7.07), 7.405 (1.32), 7.420 (6.42), 7.433 (2.80), 7.438 (2.01), 7.443 (0.95), 7.458 (1.56), 7.508 (3.80), 7.512 (6.19), 7.517 (3.44), 8.254 (8.65), 8.259 (11.49), 8.283 (9.00), 8.288 (6.84).

### Intermediate 349

### 5-bromo-3-[(1R)-1-(2-methoxyphenyl)ethoxy]-2-nitropyridine

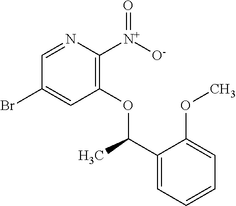 [SMILES: COc1ccccc1[C@@H](C)Oc1cc(Br)cnc1[N+](=O)[O-]]

(1R)-1-(2-Methoxyphenyl)ethan-1-ol (390 μl, 2.7 mmol) was dissolved in THF (5 mL), cooled to 0° C., sodium hydride (145 mg, 60% purity, 3.62 mmol) was added and the mixture was stirred for 30 min at 0° C. 5-Bromo-3-fluoro-2-nitropyridine (400 mg, 1.81 mmol) was dissolved in THF (4 mL) and added dropwise at 0° C. The mixture was stirred for 1 h at 0° C. and 1 h at rt. The reaction mixture was poured into water. THF was distilled off and the aqueous phase was extracted with dichloromethane. The organic phase was dried over a hydrophobic filter and concentrated. The residue was purified by column chromatography (silica gel, hexane/ethyl acetate (0-15%) gradient) to give 494 mg (97% purity, 75% yield) of the title compound.

[α]²⁰_{D}: +10.4° (c=1.00, methanol)

LC-MS (Method 2): Rₜ=1.42 min; MS (ESIpos): m/z=353 [M+H]⁺

¹H NMR (400 MHz, DMSO-d₆, 22° C.) δ ppm 1.57 (d, 3H), 3.87 (s, 3H), 6.05 (q, 1H), 6.97 (td, 1H), 7.06-7.09 (m, 1H), 7.29-7.34 (m, 2H), 7.97 (d, 1H), 8.22 (d, 1H).

### Intermediate 350

### 5-bromo-2-nitro-3-[(1R)-1-(pyridin-3-yl)ethoxy]pyridine

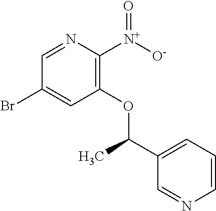 [SMILES: C[C@@H](Oc1cc(Br)cnc1[N+](=O)[O-])c1cccnc1]

(1R)-1-(Pyridin-3-yl)ethan-1-ol (111 mg, 905 μmol) was dissolved in THF (1.8 mL), cooled to 0° C., sodium hydride (39.8 mg, 60% purity, 996 μmol) was added and the mixture was stirred for 30 min at 0° C. 5-Bromo-3-fluoro-2-nitropyridine (200 mg, 905 μmol) was dissolved in THF (3 mL) and added dropwise. The mixture was stirred for 2 h at 0° C. Sodium hydride (39.8 mg, 60% purity, 996 μmol) was added again and stirred over night at room temperature. The reaction mixture was diluted with water and extracted with ethyl acetate. The combined organic layers were dried and concentrated to give 268 mg (85% purity, 78% yield) of the title compound.

LC-MS (Method 1): Rₜ=1.08 min; MS (ESIpos): m/z=324 [M+H]⁺

¹H-NMR (400 MHz, DMSO-d6) δ[ppm]: 0.822 (0.44), 0.831 (0.54), 0.851 (0.73), 1.232 (1.97), 1.600 (1.51), 1.609 (16.00), 1.625 (15.61), 1.640 (2.90), 1.657 (2.79), 2.518 (5.06), 2.523 (3.76), 2.632 (2.33), 2.679 (0.41), 6.022 (0.90), 6.038 (3.06), 6.054 (3.04), 6.070 (0.88), 6.220 (0.50), 6.236 (0.51), 7.393 (0.47), 7.405 (0.46), 7.421 (2.40), 7.423 (2.42), 7.433 (2.50), 7.435 (2.53), 7.441 (2.50), 7.444 (2.50), 7.453 (2.56), 7.455 (2.57), 7.574 (0.40), 7.814 (1.80), 7.819 (3.01), 7.823 (1.99), 7.829 (0.56), 7.834 (2.24), 7.839 (3.02), 7.844 (1.75), 7.848 (0.47), 7.854 (0.53), 8.081 (1.33), 8.086 (1.98), 8.098 (1.00), 8.104 (0.48), 8.123 (0.88), 8.128 (0.64), 8.263 (9.65), 8.267 (10.78), 8.356 (7.52), 8.360 (6.86), 8.490 (0.60), 8.494 (0.61), 8.502 (0.60), 8.506 (0.73), 8.532 (3.84), 8.536 (4.25), 8.544 (4.00), 8.548 (3.92), 8.651 (0.81), 8.656 (0.99), 8.669 (4.32), 8.673 (4.32).

### Intermediate 351

### 5-bromo-2-nitro-3-[(1R)-1-(1,3-thiazol-2-yl)ethoxy]pyridine

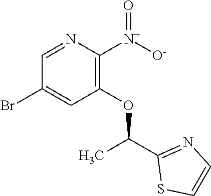 [SMILES: C[C@@H](Oc1cc(Br)cnc1[N+](=O)[O-])c1nccs1]

The compound was prepared similarly to Intermediate 335 starting from (1R)-1-(1,3-thiazol-2-yl)ethanol (421 mg, 3.2 mmol) to give 343.6 mg (100% purity, 33% yield) of the title compound.

LC-MS (Method 2): Rₜ=1.17 min; MS (ESIpos): m/z=330 [M+H]⁺

¹H-NMR (400 MHz, DMSO-d₆): δ [ppm]=1.72 (d, 3H), 6.32 (q, 1H), 7.80 (d, 1H), 7.84 (d, 1H), 8.33 (d, 1H), 8.51 (d, 1H).

### Intermediate 352

### 5-bromo-3-[(1R)-1-(3-fluorophenyl)ethoxy]-2-nitropyridine

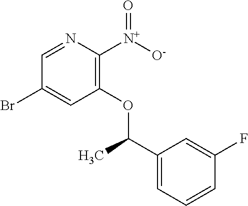 [SMILES: C[C@@H](Oc1cc(Br)cnc1[N+](=O)[O-])c1cccc(F)c1]

The compound was prepared similarly to Intermediate 335 starting from (1R)-1-(3-fluorophenyl)ethan-1-ol (360 μL, 98% purity, 3.3 mmol) to give 346 mg (100% purity, 37% yield) of the title compound.

[α]²⁰_{D}: +108.6° (c=1.00, methanol)

LC-MS (Method 1): Rₜ=1.35 min; MS (ESIpos): m/z=341 [M+H]⁺

¹H-NMR (400 MHz, DMSO-d6) δ[ppm]: 1.569 (15.99), 1.585 (16.00), 1.986 (0.51), 2.518 (0.71), 2.523 (0.47), 5.955 (0.94), 5.971 (3.16), 5.987 (3.12), 6.003 (0.91), 7.132 (0.99), 7.134 (1.08), 7.138 (1.20), 7.141 (1.23), 7.155 (2.17), 7.161 (2.41), 7.175 (1.26), 7.180 (1.35), 7.184 (1.40), 7.250 (2.46), 7.256 (4.23), 7.271 (1.91), 7.275 (5.52), 7.280 (5.67), 7.423 (1.90), 7.438 (2.29), 7.443 (2.86), 7.453 (0.53), 7.458 (2.94), 7.462 (1.63), 7.476 (0.75), 7.479 (1.07), 8.249 (7.61), 8.253 (12.59), 8.266 (9.55), 8.270 (5.84).

### Intermediate 353

### (rac)-5-bromo-2-nitro-3-[1-(1,3-oxazol-4-yl)ethoxy]pyridine (Enantiomer 1 and Enantiomer 2)

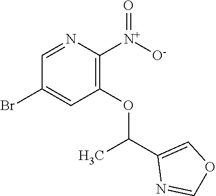 [SMILES: CC(Oc1cc(Br)cnc1[N+](=O)[O-])c1cocn1]

The compound was prepared similarly to Intermediate 335 starting from (rac)-1-(1,3-oxazol-4-yl)ethanol (270 mg, 1.2 mmol) to give 194 mg (100% purity, 51% yield) of the title compound.

LC-MS (Method 2): Rₜ=1.06 min; MS (ESIpos): m/z=314 [M+H]⁺

¹H-NMR (400 MHz, DMSO-d₆): δ [ppm]=1.61 (d, 3H), 5.93 (q, 1H), 8.23 (s, 1H), 8.28 (d, 1H), 8.39 (d, 1H), 8.50 (d, 1H).

### Intermediate 354

### (rac)-5-bromo-2-nitro-3-[1-(1,3-oxazol-2-yl)ethoxy]pyridine (Enantiomer 1 and Enantiomer 2)

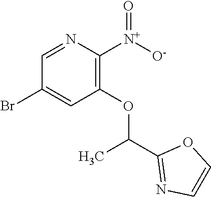 [SMILES: CC(Oc1cc(Br)cnc1[N+](=O)[O-])c1ncco1]

5-Bromo-3-fluoro-2-nitropyridine (100 mg, 453 μmol) and (rac)-1-(1,3-oxazol-2-yl)ethan-1-ol (52.7 mg, 466 μmol) were dissolved in THF (3 mL). Cesium carbonate (162 mg, 498 μmol) was added and the mixture was stirred for 4.5 h at 65° C. The reaction mixture was diluted with water and extracted with dichloromethane. The combined organic layers were dried over magnesium sulfate, filtered and concentrated to give 101 mg (85% purity, 60% yield) of the title compound.

LC-MS (Method 2): Rₜ=1.07 min; MS (ESIpos): m/z=314 [M+H]⁺

¹H-NMR (400 MHz, DMSO-d₆): δ [ppm]=1.70 (d, 3H), 6.12 (q, 1H), 7.27 (d, 1H), 8.19 (d, 1H), 8.34 (d, 1H), 8.53 (d, 1H).

### Intermediate 355

### 2-{(1R)-1-[(5-bromo-2-nitropyridin-3-yl)oxy]ethyl}pyrimidine

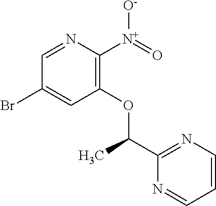 [SMILES: C[C@@H](Oc1cc(Br)cnc1[N+](=O)[O-])c1ncccn1]

The compound was prepared similarly to Intermediate 335 starting from (1R)-1-(pyrimidin-2-yl)ethan-1-ol (295 mg, 2.38 mmol) to give 688 mg (94% yield) of the title compound.

[α]²⁰_{D}: +25.1° (c=1.00, methanol)

LC-MS (Method 1): Rₜ=1.03 min; MS (ESIpos): m/z=325 [M+H]⁺

¹H NMR (DMSO-d6, 400 MHz): δ (ppm) 8.83 (d, 2H), 8.26 (d, 1H), 8.22 (d, 1H), 7.48 (t, 1H), 6.00 (q, 1H), 1.69 (d, 3H).

### Intermediate 356

### 5-bromo-2-nitro-3-[(1R)-1-(pyridin-3-yl)ethoxy]pyridine

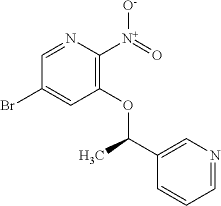 [SMILES: C[C@@H](Oc1cc(Br)cnc1[N+](=O)[O-])c1cccnc1]

5-Bromo-2-nitropyridin-3-ol (1.14 g, 5.20 mmol) was dissolved in THF (46 mL) under argon. (1S)-1-(pyridin-3-yl)ethan-1-ol (646 mg, 5.25 mmol) and triphenylphosphine (1.77 g, 6.76 mmol) were added and stirred for 1 h at room temperature. The reaction mixture was cooled to 0 to 4° C. and diisopropylazodicarboxylate (1.3 ml, 6.8 mmol) was added. The mixture was stirred over night at room temperature. The reaction mixture was concentrated to give 5.07 g (301% yield) of the title compound.

LC-MS (Method 2): Rₜ=0.89 min; MS (ESIpos): m/z=324 [M+H]⁺

¹H-NMR (400 MHz, DMSO-d6): δ [ppm]=1.62 (d, 3H), 6.05 (q, 1H), 7.41-7.47 (m, 1H), 7.83 (dt, 1H), 8.26 (d, 1H), 8.36 (d, 1H), 8.54 (dd, 1H), 8.67 (d, 1H).

### Intermediate 357

### 5-bromo-2-nitro-3-[(1R)-1-(1,2-thiazol-5-yl)ethoxy]pyridine

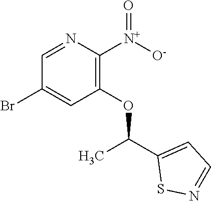 [SMILES: C[C@@H](Oc1cc(Br)cnc1[N+](=O)[O-])c1ccns1]

5-Bromo-3-fluoro-2-nitropyridine (230.0 mg, 1041 μmol) and (1R)-1-(1,2-thiazol-5-yl)ethan-1-ol (138.5 mg, 1072 μmol) were dissolved in THF (6.9 mL). Cesium carbonate (373.0 mg, 1145 μmol) was added and the mixture was stirred for 20 h at 65° C. The reaction mixture was allowed to cool down, diluted with water and extracted with dichloromethane. The combined organic layers were dried over magnesium sulfate, filtered and concentrated to give 262.0 mg (68% purity, 52% yield) of the title compound.

LC-MS (Method 2): Rₜ=1.19 min; MS (ESIpos): m/z=330 [M+H]⁺

¹H-NMR (400 MHz, DMSO-d₆): δ [ppm]=1.70 (d, 3H), 6.45 (q, 1H), 7.47 (dd, 1H), 8.32 (d, 1H), 8.47 (d, 1H), 8.53 (d, 1H).

### Intermediate 358

### (rac)-5-bromo-3-[1-(2,6-dichlorophenyl)ethoxy]-2-nitropyridine

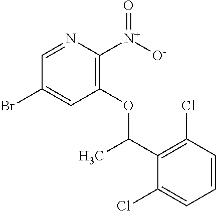 [SMILES: CC(Oc1cc(Br)cnc1[N+](=O)[O-])c1c(Cl)cccc1Cl]

(Rac)1-(2,6-Dichlorophenyl)ethan-1-ol (500 mg, 2.62 mmol) was dissolved in THF (24.5 mL) and DMF, cooled to 0° C., sodium hydride (110 mg, 60% purity, 2.74 mmol) was added and stirred for 30 min at 0° C. A solution of 5-bromo-3-fluoro-2-nitropyridine (551 mg, 2.49 mmol) in THF (0.5 mL) was added and the mixture was stirred for 2 h at 0° C. and over night at room temperature. The reaction mixture was quenched with saturated ammonium chloride solution and extracted with ethyl acetate. The combined organic layers were washed with brine, dried over sodium sulfate, filtered and concentrated to give 1.04 g (107% yield) of the title compound.

LC-MS (Method 1): Rₜ=1.45 min; MS (ESIpos): m/z=393 [M+H]⁺

¹H-NMR (400 MHz, DMSO-d6) δ[ppm]: 1.152 (1.57), 1.170 (3.38), 1.188 (1.75), 1.226 (0.69), 1.350 (1.86), 1.447 (2.88), 1.464 (2.94), 1.722 (1.46), 1.739 (2.47), 1.744 (16.00), 1.754 (1.44), 1.761 (15.95), 1.986 (5.94), 2.518 (1.19), 2.522 (0.78), 3.998 (0.41), 4.015 (1.27), 4.033 (1.26), 4.051 (0.40), 6.353 (0.95), 6.370 (3.55), 6.386 (3.52), 6.403 (0.94), 7.235 (0.46), 7.253 (0.63), 7.256 (0.60), 7.275 (0.84), 7.319 (0.49), 7.370 (3.50), 7.385 (2.06), 7.389 (3.54), 7.392 (3.82), 7.404 (1.24), 7.411 (5.96), 7.419 (0.97), 7.439 (0.56), 7.508 (12.09), 7.528 (7.13), 7.770 (0.54), 7.779 (6.13), 7.784 (6.22), 7.979 (0.73), 7.984 (0.74), 8.077 (0.48), 8.262 (8.00), 8.267 (7.80).

### Intermediate 359

### 5-bromo-3-{[1-(3-{3-methyl-3-[(-oxan-2-yl)oxy]but-1-yn-1-yl}phenyl)ethyl]oxy}-2-nitropyridine (Mixture of 4 Isomers)

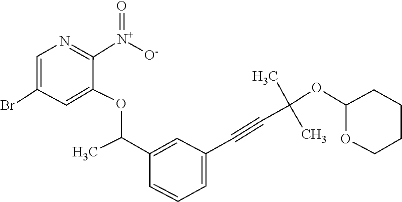 [SMILES: CC(Oc1cc(Br)cnc1[N+](=O)[O-])c1cccc(C#CC(C)(C)OC2CCCCO2)c1]

Sodium hydride (106 mg, 60% purity, 2.65 mmol) was suspended in THF (5 mL) and cooled to 0° C. 1-(3-{3-Methyl-3-[(oxan-2-yl)oxy]but-1-yn-1-yl}phenyl)ethan-1-ol (267 mg, 926 μmol) was dissolved in THF (1.5 mL) and added. The mixture was stirred for 30 min at 0° C. A solution of 5-bromo-3-fluoro-2-nitropyridine (195 mg, 882 μmol) in THF (1.5 mL) was added and stirred for 3 h at 0° C. The reaction mixture was quenched with saturated ammonium chloride solution and extracted with ethyl acetate. The combined organic layers were dried and concentrated. The residue was purified by column chromatography (silica gel, hexane/ethyl acetate (0-20%) gradient) followed by column chromatography (silica gel NH2, hexane/ethyl acetate (0-20%) gradient) to give 2.08 g (74% yield) of the title compound.

LC-MS (Method 1): Rₜ=1.60 min; MS (ESIpos): m/z=489 [M+H]⁺

¹H-NMR (400 MHz, DMSO-d6) δ[ppm]: 1.084 (0.40), 1.154 (2.45), 1.172 (4.88), 1.190 (2.38), 1.232 (0.50), 1.259 (0.57), 1.406 (0.57), 1.425 (1.21), 1.435 (1.76), 1.448 (1.98), 1.464 (1.62), 1.475 (1.36), 1.485 (1.10), 1.513 (16.00), 1.545 (12.17), 1.558 (7.79), 1.574 (7.43), 1.606 (0.45), 1.615 (0.52), 1.628 (0.55), 1.642 (0.86), 1.659 (0.40), 1.671 (0.62), 1.710 (1.02), 1.720 (0.57), 1.729 (0.74), 1.739 (0.74), 1.749 (0.62), 1.759 (0.55), 1.987 (8.40), 2.318 (0.43), 2.518 (5.45), 2.523 (3.40), 2.660 (0.40), 3.419 (0.64), 3.435 (0.86), 3.447 (0.93), 3.463 (0.64), 3.826 (0.69), 3.834 (0.64), 3.843 (0.93), 3.853 (0.93), 3.862 (0.57), 3.872 (0.60), 4.000 (0.62), 4.017 (1.93), 4.035 (1.93), 4.053 (0.62), 5.074 (0.86), 5.080 (1.33), 5.085 (1.52), 5.092 (1.14), 5.098 (0.81), 5.940 (0.50), 5.956 (1.76), 5.972 (1.74), 5.988 (0.48), 7.347 (0.74), 7.352 (0.88), 7.354 (0.98), 7.357 (0.95), 7.364 (1.31), 7.370 (1.93), 7.374 (1.76), 7.389 (1.17), 7.407 (3.14), 7.417 (1.98), 7.423 (4.19), 7.440 (0.50), 7.487 (2.93), 8.248 (4.36), 8.252 (6.55), 8.268 (3.40).

### Intermediate 360

### (rac)-7-[(5-bromo-2-nitropyridin-3-yl)oxy]-6,7-dihydro-5H-cyclopenta[b]pyridine

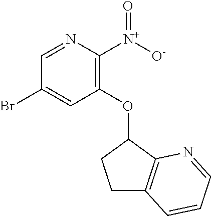 [SMILES: O=[N+]([O-])c1ncc(Br)cc1OC1CCc2cccnc21]

5-Bromo-2-nitropyridin-3-ol (100 mg, 457 μmol) was dissolved in THF (4.2 mL) under argon and (rac)-6,7-dihydro-5H-cyclopenta[b]pyridin-7-ol (61.7 mg, 457 μmol) and triphenylphosphine (934 mg, 3.5 mmol) were added and stirred for 1 h at room temperature. The reaction mixture was cooled to 0 to 4° C., diisopropyl azodicarboxylate (120 μL, 590 μmol) was added and stirred for 1.5 h at 4° C. and over night at room temperature. The mixture was diluted with aqueous potassium carbonate solution and extracted with ethyl acetate. The organic phase was dried and concentrated. The residue was purified by column chromatography (silica gel, hexane/ethyl acetate (0-40%) gradient). The combined reactions were concentrated and the residue was treated with MTBE. The crude product was filtered and the filtrate was concentrated to give 120 mg (78% yield) of the title compound.

LC-MS (Method 1): Rₜ=1.18 min; MS (ESIpos): m/z=336 [M+H]⁺

¹H NMR (DMSO-d₆, 400 MHz): δ (ppm) 8.78 (d, 1H), 8.43-8.47 (m, 1H), 8.32 (d, 1H), 7.81 (dd, 1H), 7.36 (dd, 1H), 6.21 (dd, 1H), 3.01-3.12 (m, 1H), 2.87-2.96 (m, 1H), 2.56-2.69 (m, 1H), 2.17 (dddd, 1H).

### Intermediate 361

### (rac)-5-bromo-3-[1-(4,5-dimethyl-1,3-thiazol-2-yl)ethoxy]-2-nitropyridine

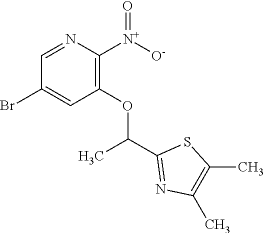 [SMILES: Cc1nc(C(C)Oc2cc(Br)cnc2[N+](=O)[O-])sc1C]

Sodium hydride (10.9 mg, 60% purity, 272 μmol) was added to (rac)-1-(4,5-dimethyl-1,3-thiazol-2-yl)ethan-1-ol (36.6 mg, 233 μmol) in THF (2 mL) under argon at 0° C. The mixture was stirred for 1 h at 0° C. A solution of 5-bromo-3-fluoro-2-nitropyridine (50.0 mg, 226 μmol) in THF was added dropwise at 0° C. and stirred for 1.5 h at 0° C. The reaction mixture was diluted with water and extracted with dichloromethane. The organic phase was dried over magnesium sulfate, filtered and concentrated. The residue was purified by preparative HPLC to give 42.0 mg (92% purity, 48% yield) of the title compound.

LC-MS (Method 1): Rₜ=1.34 min; MS (ESIpos): m/z=358 [M+H]⁺

¹H-NMR (400 MHz, DMSO-d₆): δ [ppm]=1.66 (d, 3H), 2.24 (s, 3H), 2.31 (s, 3H), 6.16 (q, 1H), 8.32 (d, 1H), 8.48 (d, 1H).

### Intermediate 362

### (rac)-5-bromo-3-[1-(5-methyl-1,3-thiazol-2-yl)ethoxy]-2-nitropyridine

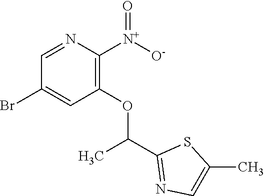 [SMILES: Cc1cnc(C(C)Oc2cc(Br)cnc2[N+](=O)[O-])s1]

(Rac)-1-(5-Methyl-1,3-thiazol-2-yl)ethan-1-ol (167 mg, 1.17 mmol) was dissolved in THF (5 mL), cooled to 0° C. and sodium hydride (54.3 mg, 60% purity, 1.36 mmol) was added. The mixture was stirred for 1 h at 0° C. A solution of 5-bromo-3-fluoro-2-nitropyridine (250 mg, 1.13 mmol) in THF (5 mL) was added dropwise and stirred for 3 h at 0° C. The reaction mixture was diluted with water and extracted with dichloromethane. The combined organic layers were dried over magnesium sulfate, filtered and concentrated. The residue was purified by preparative HPLC to give 225 mg (100% purity, 58% yield) of the title compound.

LC-MS (Method 2): Rₜ=1.27 min; MS (ESIpos): m/z=344 [M+H]⁺

¹H-NMR (400 MHz, DMSO-d₆): δ [ppm]=1.68 (d, 3H), 2.43 (d, 3H), 6.21 (q, 1H), 7.50 (q, 1H), 8.32 (d, 1H), 8.48 (d, 1H).

### Intermediate 363

### (rac)-5-bromo-2-nitro-3-{[1-(1,3-thiazol-4-yl)ethyl]oxy}pyridine

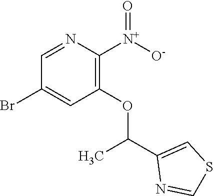 [SMILES: CC(Oc1cc(Br)cnc1[N+](=O)[O-])c1cscn1]

Sodium hydride (39.1 mg, 1.63 mmol, 60% in oil) was added to (rac)-1-(1,3-thiazol-4-yl)ethan-1-ol (181 mg, 1.40 mmol) in THF (4.5 mL) at 0° C. The mixture was stirred for 1 h at 0° C. A solution of 5-bromo-3-fluoro-2-nitropyridine (300 mg, 1.36 mmol) in THF (4.5 mL) was added dropwise at 0° C. and stirred over night and allowed to warm up to rt. (Rac)-1-(1,3-thiazol-4-yl)ethan-1-ol (90 mg, 0.70 mmol) was dissolved in THF, cooled to 0° C., sodium hydride (19.5 mg, 0.81 mmol, 60% in oil) was added and stirred for 1 h at 0° C. This reaction mixture was added to the first reaction mixture at 0° C. and stirred for 1 h at 0° C. The reaction mixture was diluted with water and extracted with dichloromethane. The combined organic layers were washed with brine, dried over a hydrophobic filter and concentrated. The residue was purified by preparative HPLC to give 239 mg (100% purity, 53% yield) of the title compound.

LC-MS (Method 2): Rₜ=1.13 min; MS (ESIpos): m/z=330 [M+H]⁺

¹H-NMR (400 MHz, DMSO-d₆): δ [ppm]=1.67 (d, 3H), 6.10 (q, 1H), 7.80 (d, 1H), 8.27 (d, 1H), 8.44 (d, 1H), 9.12 (d, 1H).

### Intermediate 364

### 5-bromo-3-[(1R)-1-(5-methyl-1,3,4-oxadiazol-2-yl)ethoxy]-2-nitropyridine

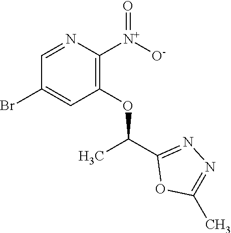 [SMILES: Cc1nnc([C@@H](C)Oc2cc(Br)cnc2[N+](=O)[O-])o1]

(1R)-1-(5-Methyl-1,3,4-oxadiazol-2-yl)ethan-1-ol (133 mg, 1.04 mmol) was dissolved in THF (4 mL), cooled to 0° C. and sodium hydride (27.5 mg, 1.15 mmol, 60% in oil) was added. The mixture was stirred for 1 h at 0° C. A solution of 5-bromo-3-fluoro-2-nitropyridine (200 mg, 905 μmol) in THF (4 mL) was added dropwise at 0° C. and stirred for 17 h at 0° C. The reaction mixture was diluted with water and extracted with dichloromethane. The combined organic layers were dried over magnesium sulfate, filtered and concentrated. The residue was purified by preparative HPLC to give 167 mg (86% purity, 48% yield) of the title compound.

LC-MS (Method 2): Rₜ=0.98 min; MS (ESIpos): m/z=329 [M+H]⁺

¹H-NMR (400 MHz, DMSO-d₆): δ [ppm]=1.71 (d, 3H), 6.21 (q, 1H), 8.37 (d, 1H), 8.56 (d, 1H).

### Intermediate 365

### 5-bromo-3-[(1R)-1-(2-methyl-1,3-thiazol-4-yl)ethoxy]-2-nitropyridine

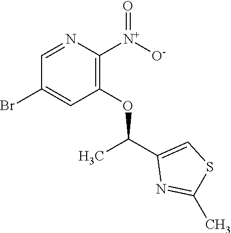 [SMILES: Cc1nc([C@@H](C)Oc2cc(Br)cnc2[N+](=O)[O-])cs1]

(1R)-1-(2-Methyl-1,3-thiazol-4-yl)ethan-1-ol (171 mg, 1.19 mmol) was dissolved in THF (7 mL), cooled to 0° C. and sodium hydride (47.8 mg, 1.99 mmol, 60% in oil) was added. The mixture was stirred for 1 h at 0° C. A solution of 5-bromo-3-fluoro-2-nitropyridine (220 mg, 996 μmol) in THF (7 mL) was added dropwise at 0° C. and stirred over night to reach slowly rt. The reaction mixture was diluted with water and extracted with dichloromethane. The combined organic layers were dried over a hydrophobic filter and concentrated. The residue was purified by preparative HPLC to give 183 mg (98% purity, 52% yield) of the title compound.

LC-MS (Method 2): Rₜ=1.23 min; MS (ESIpos): m/z=344 [M+H]⁺

¹H-NMR (400 MHz, DMSO-d₆): δ [ppm]=1.63 (d, 3H), 2.63 (s, 3H), 5.98 (q, 1H), 7.55 (s, 1H), 8.27 (d, 1H), 8.43 (d, 1H).

### Intermediate 366

### (rac)-5-bromo-3-[1-(4-methyl-1,3-thiazol-2-yl)ethoxy]-2-nitropyridine

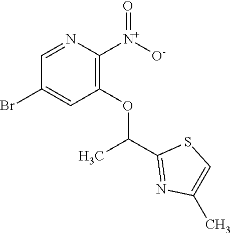 [SMILES: Cc1csc(C(C)Oc2cc(Br)cnc2[N+](=O)[O-])n1]

The compound was prepared similarly to Intermediate 335 starting from (rac)-1-(4-methyl-1,3-thiazol-2-yl)ethan-1-ol (33.4 mg, 233 μmol) to give 48.0 mg (100% purity, 62% yield) of the title compound.

LC-MS (Method 2): Rₜ=1.24 min; MS (ESIpos): m/z=344 [M+H]⁺

¹H-NMR (400 MHz, DMSO-d₆): δ [ppm]=1.69 (d, 3H), 2.35 (d, 3H), 6.24 (q, 1H), 7.32 (q, 1H), 8.33 (d, 1H), 8.50 (d, 1H).

### Intermediate 367

### (rac)-5-bromo-3-{1-[3-(methanesulfonyl)phenyl]ethoxy}-2-nitropyridine

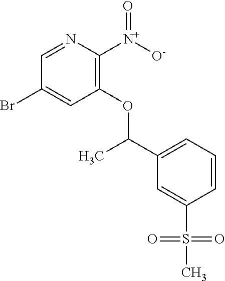 [SMILES: CC(Oc1cc(Br)cnc1[N+](=O)[O-])c1cccc(S(C)(=O)=O)c1]

The compound was prepared similarly to Intermediate 335 starting from (rac)-1-[3-(methanesulfonyl)phenyl]ethan-1-ol (800 mg, 3.99 mmol) to give 2.22 g (145% yield) of the title compound.

LC-MS (Method 1): Rₜ=1.13 min; MS (ESIneg): m/z=399 [M−H]⁻

¹H-NMR (400 MHz, DMSO-d6) δ[ppm]: 1.150 (1.67), 1.168 (3.16), 1.187 (1.50), 1.344 (1.86), 1.350 (1.28), 1.361 (1.86), 1.612 (4.86), 1.627 (4.84), 1.639 (0.55), 1.656 (0.45), 1.735 (0.43), 1.751 (1.23), 1.985 (6.29), 2.153 (0.53), 3.204 (4.57), 3.222 (16.00), 3.228 (1.91), 3.239 (0.43), 3.594 (0.94), 3.611 (0.43), 3.996 (0.41), 4.014 (1.28), 4.032 (1.28), 4.050 (0.41), 6.102 (1.07), 6.118 (1.06), 7.594 (0.55), 7.679 (1.01), 7.698 (2.29), 7.718 (1.59), 7.768 (0.85), 7.771 (1.59), 7.774 (1.25), 7.787 (0.61), 7.791 (1.03), 7.794 (0.83), 7.883 (0.90), 7.886 (1.12), 7.888 (1.14), 7.891 (0.92), 7.903 (1.08), 7.907 (1.50), 7.910 (1.00), 8.033 (1.28), 8.038 (2.17), 8.042 (1.13), 8.079 (0.40), 8.260 (3.41), 8.265 (3.74), 8.357 (2.73), 8.362 (2.51), 8.678 (0.46).

### Intermediate 368

### (rac)-5-bromo-2-nitro-3-[1-(1,3-oxazol-4-yl)ethoxy]pyridine

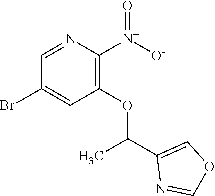 [SMILES: CC(Oc1cc(Br)cnc1[N+](=O)[O-])c1cocn1]

The compound was prepared similarly to Intermediate 335 starting from (rac)-1-(1,3-oxazol-4-yl)ethan-1-ol (142 mg, 1.26 mmol) to give 194 mg (100% purity, 51% yield) of the title compound.

LC-MS (Method 2): Rₜ=1.06 min; MS (ESIpos): m/z=314 [M+H]⁺

¹H-NMR (400 MHz, DMSO-d₆): δ [ppm]=1.61 (d, 3H), 5.93 (q, 1H), 8.23 (s, 1H), 8.28 (d, 1H), 8.39 (d, 1H), 8.50 (d, 1H).

### Intermediate 369

### (rac)-5-bromo-2-nitro-3-[1-(1-propyl-1H-pyrazol-5-yl)ethoxy]pyridine

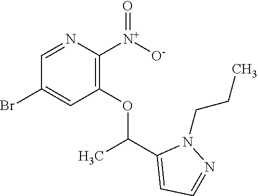 [SMILES: CCCn1nccc1C(C)Oc1cc(Br)cnc1[N+](=O)[O-]]

The compound was prepared similarly to Intermediate 335 starting from (rac)-1-(1-propyl-1H-pyrazol-5-yl)ethan-1-ol (355 mg, 87% purity, 2.00 mmol) to give 697 mg (103% yield) of the title compound.

LC-MS (Method 1): Rₜ=1.21 min; MS (ESIpos): m/z=355 [M+H]⁺

¹H-NMR (400 MHz, DMSO-d6) δ[ppm]: 0.744 (2.16), 0.762 (5.00), 0.769 (6.73), 0.781 (2.91), 0.788 (16.00), 0.806 (7.55), 0.825 (3.23), 0.831 (0.68), 0.843 (6.31), 0.851 (0.82), 0.862 (3.23), 1.233 (1.60), 1.399 (5.68), 1.414 (6.24), 1.619 (11.29), 1.635 (11.27), 1.657 (3.68), 1.672 (3.57), 1.683 (0.75), 1.702 (2.05), 1.720 (3.20), 1.738 (3.05), 1.752 (2.66), 1.756 (1.71), 1.770 (2.20), 1.789 (1.03), 2.518 (3.43), 2.523 (2.56), 3.990 (0.57), 3.999 (0.46), 4.009 (0.71), 4.019 (3.77), 4.025 (1.25), 4.038 (5.33), 4.056 (4.90), 4.075 (0.82), 4.090 (0.43), 4.784 (0.51), 4.799 (0.74), 4.815 (0.52), 5.231 (1.60), 5.246 (1.52), 5.758 (1.63), 6.110 (1.63), 6.114 (1.65), 6.152 (0.65), 6.167 (2.23), 6.183 (2.22), 6.199 (0.65), 6.364 (5.57), 6.368 (6.01), 6.382 (2.51), 6.386 (1.85), 7.293 (1.71), 7.297 (1.71), 7.395 (1.51), 7.400 (1.42), 7.428 (5.34), 7.433 (5.44), 8.123 (0.92), 8.129 (1.08), 8.148 (0.77), 8.153 (1.42), 8.163 (2.99), 8.169 (1.88), 8.314 (7.08), 8.318 (7.38), 8.478 (4.99), 8.482 (4.74), 8.678 (1.00), 8.682 (1.17), 8.737 (0.95), 8.742 (0.79), 8.763 (0.83), 8.767 (0.85).

### Intermediate 370

### (rac)-4-{1-[(5-bromo-2-nitropyridin-3-yl)oxy]ethyl}pyrimidine

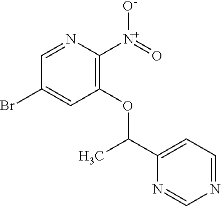 [SMILES: CC(Oc1cc(Br)cnc1[N+](=O)[O-])c1ccncn1]

The compound was prepared similarly to Intermediate 335 starting from (rac)-1-(pyrimidin-4-yl)ethan-1-ol (590 mg, 4.75 mmol) to give 1.13 g (77% yield) of the title compound.

LC-MS (Method 1): Rₜ=1.01 min; MS (ESIpos): m/z=325 [M+H]⁺

¹H NMR (400 MHz, DMSO-d₆) δ ppm 1.63 (d, 3H) 5.97 (q, 1H) 7.60 (dd, 1H) 8.32 (d, 1H), 8.34 (d, 1H), 8.88 (d, 1H) 9.20 (d, 1H).

### Intermediate 371

### (rac)-5-{1-[(5-bromo-2-nitropyridin-3-yl)oxy]ethyl}-2,4-dimethylpyrimidine

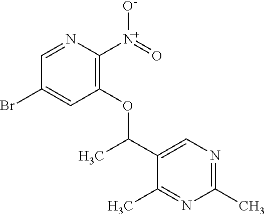 [SMILES: Cc1ncc(C(C)Oc2cc(Br)cnc2[N+](=O)[O-])c(C)n1]

The compound was prepared similarly to Intermediate 335 starting from (rac)-1-(2,4-dimethylpyrimidin-5-yl)ethan-1-ol (434 mg, 2.85 mmol)) to give 860 mg (90% yield) of the title compound.

LC-MS (Method 1): Rₜ=1.04 min; MS (ESIpos): m/z=353 [M+H]⁺

¹H NMR (DMSO-de, 400 MHz): δ (ppm) 8.60 (s, 1H), 8.41 (d, 1H), 8.31 (d, 1H), 6.12 (q, 1H), 2.56 (s, 3H), 2.52 (s, 3H), 1.60 (d, 3H).

### Intermediate 372 and Intermediate 373

### 5-{1-[(5-bromo-2-nitropyridin-3-yl)oxy]ethyl}-2,4-dimethylpyrimidine (Enantiomer 1 and Enantiomer 2)

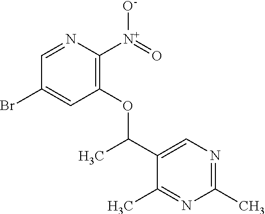 [SMILES: Cc1ncc(C(C)Oc2cc(Br)cnc2[N+](=O)[O-])c(C)n1]

The title compound from Intermediate 371 was separated into enantiomers by preparative chiral HPLC to give 426.8 mg (99% purity, 42% yield) of the title compound (enantiomer 1) and 413.3 mg (99% purity, 41% yield) of the title compound (enantiomer 2).

Preparative Chiral HPLC Method:

Instrument: Sepiatec: Prep SFC100; Column: Chiralpak IG 5μ, 250×50; eluent A: CO2; eluent B: 2-propanol+0.4 vol % diethylamine; isocratic: 10% B; flow: 100 mL/min; temperature: 40° C.; BPR: 150 bar; UV: 254 nm

Analytical Chiral HPLC Method:

Instrument: Agilent: 1260, Aurora SFC-Modul; Column: Chiralpak IG 5μ, 100×4.6; eluent A: CO2; eluent B: 2-propanol+0.4 vol % diethylamine; isocratic: 10% B; flow: 4 mL/min; temperature: 37.5° C.; BPR: 100 bar; UV: 254 nm

Enantiomer 1:

Retention time preparative method: 9.25-11.50 min

Retention time analytical method: 3.07 min

[α]²⁰_{D}: −104.2° (c=1.00, methanol)

¹H NMR (DMSO-d₆, 400 MHz): δ (ppm) 8.60 (s, 1H), 8.41 (d, 1H), 8.31 (d, 1H), 6.12 (q, 1H), 2.55 (s, 3H), 2.52 (s, 3H), 1.60 (d, 3H).

Enantiomer 2:

Retention time preparative method: 12.75-17.00 min

Retention time analytical method: 4.30 min

[α]²⁰_{D}: +94.5° (c=1.00, methanol)

¹H NMR (DMSO-d₆, 400 MHz): δ (ppm) 8.60 (s, 1H), 8.41 (d, 1H), 8.31 (d, 1H), 6.12 (q, 1H), 2.55 (s, 3H), 2.52 (s, 3H), 1.60 (d, 3H).

### Intermediate 374

### (rac)-5-bromo-3-{1-[4-(methanesulfonyl)phenyl]ethoxy}-2-nitropyridine

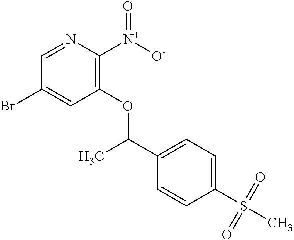 [SMILES: CC(Oc1cc(Br)cnc1[N+](=O)[O-])c1ccc(S(C)(=O)=O)cc1]

The compound was prepared similarly to Intermediate 335 starting from (rac)-1-[4-(methanesulfonyl)phenyl]ethan-1-ol (237.8 mg, 1.18 mmol) to give 503 mg (110% yield) of the title compound.

LC-MS (Method 1): Rₜ=1.13 min; MS (ESIneg): m/z=399 [M−H]⁻

¹H-NMR (400 MHz, DMSO-d6) δ[ppm]: 1.153 (0.46), 1.171 (1.02), 1.189 (0.57), 1.229 (0.68), 1.299 (5.02), 1.315 (5.26), 1.352 (3.07), 1.565 (16.00), 1.581 (15.95), 1.595 (3.88), 1.611 (3.83), 1.986 (1.70), 2.518 (1.53), 2.523 (1.07), 5.936 (0.95), 5.952 (3.24), 5.968 (3.21), 5.984 (0.91), 6.183 (0.69), 6.199 (0.68), 7.200 (0.56), 7.217 (0.50), 7.274 (0.79), 7.279 (0.78), 7.288 (0.56), 7.292 (1.17), 7.295 (2.03), 7.299 (2.42), 7.305 (1.07), 7.312 (3.09), 7.318 (2.52), 7.320 (1.92), 7.325 (3.26), 7.329 (3.97), 7.334 (2.23), 7.340 (0.67), 7.346 (1.06), 7.350 (1.85), 7.363 (0.43), 7.368 (1.59), 7.372 (3.25), 7.376 (1.59), 7.388 (2.70), 7.391 (8.17), 7.404 (2.96), 7.409 (9.17), 7.414 (8.49), 7.418 (10.51), 7.429 (1.29), 7.435 (3.01), 7.440 (2.06), 8.066 (1.33), 8.071 (2.64), 8.075 (1.65), 8.080 (0.51), 8.100 (1.02), 8.105 (0.89), 8.222 (5.12), 8.226 (14.87), 8.230 (11.55), 8.235 (3.96), 8.240 (0.44).

### Intermediate 375

### (rac)-5-bromo-3-[1-(1-methyl-1H-1,2,3-triazol-4-yl)ethoxy]-2-nitropyridine

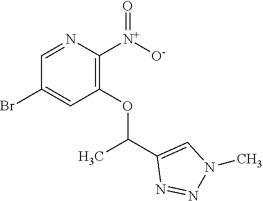 [SMILES: CC(Oc1cc(Br)cnc1[N+](=O)[O-])c1cn(C)nn1]

The compound was prepared similarly to Intermediate 335 starting from (rac)-1-(1-methyl-1H-1,2,3-triazol-4-yl)ethan-1-ol (138 mg, 1.09 mmol) to yield 319 mg (107% yield) of the title product which was used without further purification in the next step.

LC-MS (Method 2): Rₜ=0.99 min; MS (ESIpos): m/z=328 [M+H]⁺

¹H-NMR (400 MHz, DMSO-d6): δ [ppm]=1.66 (d, 3H), 4.03 (s, 3H), 6.08 (q, 1H), 8.20 (s, 1H), 8.27 (d, 1H), 8.52 (d, 1H).

Reduction+Chiral Sep

### Intermediate 376 and Intermediate 377

### 5-bromo-3-[1-(6-methylpyridin-2-yl)ethoxy]pyridin-2-amine (Enantiomer 1 and Enantiomer 2)

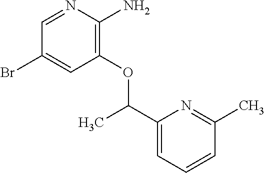 [SMILES: Cc1cccc(C(C)Oc2cc(Br)cnc2N)n1]

To a suspension of (rac)-5-bromo-3-{[−1-(6-methylpyridin-2-yl)ethyl]oxy}-2-nitropyridine (Intermediate 325, 440 mg, 65% purity, 846 μmol) in ethanol (4.5 mL) under argon were added iron (189 mg, 3.38 mmol) and acetic acid (2.3 mL). The mixture was stirred for 3 h at 80° C. The reaction mixture was then cooled to rt, diluted with dichloromethane and filtered. The filtrate was washed with 2M hydrochloric acid. The aqueous layer was extracted with dichloromethane, basified with 2M sodium hydroxide solution and extracted with ethyl acetate. The organic layer was washed with sat. sodium chloride solution, dried over sodium sulfate, filtered and concentrated under reduced pressure. The crude material was purified by flash column chromatography (silica gel, hexane/ethyl acetate (0-80%) gradient) and chiral HPLC to give the title compounds.

LC-MS (Method 1): Rₜ=1.05 min; MS (ESIpos): m/z=308 [M+H]⁺

¹H-NMR (400 MHz, CHLOROFORM-d): δ [ppm]=1.70 (d, 3H), 2.58 (s, 3H), 4.82 (br s, 2H), 5.30 (q, 1H), 6.90 (d, 1H), 7.09 (dd, 2H), 7.56 (t, 1H), 7.66 (d, 1H).

Chiral Separation:

Preparative

Instrument: Sepiatec: Prep SFC100; Column: Chiralpak IG 5μ 250×30 mm; eluent A: CO2; eluent B: methanol+0.2 vol % aqueous ammonia (32%); isocratic: 10% B; flow: 100 mL/min; temperature: 40° C.; BPR: 150 bar; UV: 254 nm

Analytical

Instrument: Waters Acquity UPLCMS SingleQuad; Column: Acquity UPLC BEH C18 1.7μ, 50×2.1 mm; eluent A: water+0.2 vol % aqueous ammonia (32%); eluent B: acetonitrile; gradient: 0-1.6 min 1-99% B, 1.6-2.0 min 99% B; flow: 0.8 mL/min; temperature: 60° C.; DAD scan: 210-400 nm

Enantiomer 1:

98 mg (37% yield)

Retention time preparative method: 7.5-10.5 min

Retention time analytical method: 2.35 min

[α]²⁰_{D}: +38.9° (c=1.00, MeOH)

Enantiomer 2:

106 mg (38% yield)

Retention time preparative method: 11.5-15.0 min

Retention time analytical method: 3.26 min

[α]²⁰_{D}: −32.0° (c=1.00, MeOH)

### Intermediate 378 and Intermediate 379

### 5-bromo-3-[1-(5-methylpyridin-2-yl)ethoxy]pyridin-2-amine (Enantiomer 1 and Enantiomer 2)

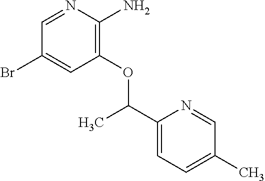 [SMILES: Cc1ccc(C(C)Oc2cc(Br)cnc2N)nc1]

The title compounds were prepared similarly to Intermediate 376 and Intermediate 377 starting from (rac)-5-bromo-3-{[1-(5-methylpyridin-2-yl)ethyl]oxy}-2-nitropyridine (490 mg, 70% purity, 1.01 mmol).

LC-MS (Method 1): Rₜ=1.05 min; MS (ESIpos): m/z=308 [M+H]⁺

¹H-NMR (400 MHz, CHLOROFORM-d): δ [ppm]=1.69 (d, 3H), 2.33 (s, 3H), 4.80 (br s, 2H), 5.32 (q, 1H), 6.88 (d, 1H), 7.21 (d, 1H), 7.48 (dd, 1H), 7.65 (d, 1H), 8.40-8.42 (m, 1H).

Chiral Separation:

Preparative

Instrument: PrepCon Labomatic HPLC-1; Column: YMC Cellulose SB 5μ, 250×30; eluent A: hexane+0.1 vol % diethylamine; eluent B: ethanol+0.1 vol % diethylamine; isocratic: 95% A+5% B; flow: 50 mL/min; temperature: 25° C.; UV: 254 nm

Analytical

Instrument: Thermo Fisher UltiMate 3000; Column: YMC Cellulose SB 3μ, 100×4.6; eluent A: hexane+0.1 vol % diethylamine; eluent B: ethanol; isocratic: 95% A+5% B; flow: 1.4 mL/min; temperature: 25° C.; UV: 254 nm

Enantiomer 1:

75.9 mg (24% yield)

Retention time preparative method: 8.3-9.0 min

Retention time analytical method: 2.42 min

[α]²⁰_{D}: −37.1° (c=1.00, MeOH)

Enantiomer 2:

80.2 mg (25% yield)

Retention time preparative method: 9.4-10.3 min

Retention time analytical method: 2.75 min

[α]²⁰_{D}: +40.5° (c=1.00, MeOH)

### Intermediate 380 and Intermediate 381

### 5-bromo-3-[1-(4-methylpyridin-2-yl)ethoxy]pyridin-2-amine (Enantiomer 1 and Enantiomer 2)

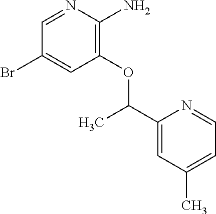 [SMILES: Cc1ccnc(C(C)Oc2cc(Br)cnc2N)c1]

The title compounds were prepared similarly to Intermediate 376 and Intermediate 377 starting from (rac)-5-bromo-3-{[1-(4-methylpyridin-2-yl)ethyl]oxy}-2-nitropyridine (466 mg, 57% purity, 785 μmol).

LC-MS (Method 1): Rₜ=1.03 min; MS (ESIpos): m/z=308 [M+H]⁺

¹H-NMR (400 MHz, CHLOROFORM-d): δ [ppm]=1.69 (d, 3H), 2.33 (s, 3H), 4.81 (br s, 2H), 5.30 (q, 1H), 6.88 (d, 1H), 7.03-7.06 (m, 1H), 7.13 (s, 1H), 7.65-7.69 (m, 1H), 8.44 (d, 1H).

Chiral Separation:

Preparative

Instrument: PrepCon Labomatic HPLC-1; Column: YMC Cellulose SB 5μ, 250×30; eluent A: hexane+0.1 vol % diethylamine; eluent B: ethanol+0.1 vol % diethylamine; isocratic: 95% A+5% B; flow: 50 mL/min; temperature: 25° C.; UV: 254 nm

Analytical

Instrument: Thermo Fisher UltiMate 3000; Column: YMC Cellulose SB 3μ, 100×4.6; eluent A: hexane+0.1 vol % diethylamine; eluent B: ethanol; isocratic: 95% A+5% B; flow: 1.4 mL/min; temperature: 25° C.; UV: 254 nm

Enantiomer 1:

54.7 mg (22% yield)

(Retention time preparative method: 8.5-9.3 min

Retention time analytical method: 2.46 min

[α]²⁰_{D}: −34.1° (c=1.00, MeOH)

Enantiomer 2:

(53.6 mg (21% yield)

Retention time preparative method: 9.5-10.2 min

Retention time analytical method: 2.76 min

[α]²⁰_{D}: +35.7° (c=1.00, MeOH)

### Intermediate 382 and Intermediate 383

### 5-bromo-3-[1-(3-methylpyridin-2-yl)ethoxy]pyridin-2-amine (Enantiomer 1 and Enantiomer 2)

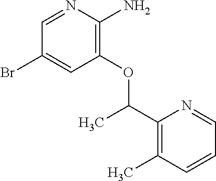 [SMILES: Cc1cccnc1C(C)Oc1cc(Br)cnc1N]

The title compounds were prepared similarly to Intermediate 376 and Intermediate 377 starting from (rac)-bromo-3-{[1-(3-methylpyridin-2-yl)ethyl]oxy}-2-nitropyridine (240 mg, 74% purity, 525 μmol)

LC-MS (Method 1): Rₜ=1.02 min; MS (ESIpos): m/z=308 [M+H]⁺

¹H-NMR (400 MHz, CHLOROFORM-d): δ [ppm]=1.74 (d, 3H), 2.38 (s, 3H), 4.79 (br s, 2H), 5.51 (q, 1H), 6.86 (d, 1H), 7.16 (dd, 1H), 7.45-7.49 (m, 1H), 7.66 (d, 1H), 8.46 (dd, 1H).

Chiral Separation:

Preparative

Instrument: PrepCon Labomatic HPLC-1; Column: YMC Cellulose SB 5μ, 250×30; eluent A: hexane+0.1 vol % diethylamine; eluent B: ethanol+0.1 vol % diethylamine; isocratic: 95% A+5% B; flow: 50 mL/min; temperature: 25° C.; UV: 254 nm

Analytical

Instrument: Thermo Fisher UltiMate 3000; Column: YMC Cellulose SB 3μ, 100×4.6; eluent A: hexane+0.1 vol % diethylamine; eluent B: ethanol; isocratic: 95% A+5% B; flow: 1.4 mL/min; temperature: 25° C.; UV: 254 nm

Enantiomer 1:

34.4 mg (21% yield)

Retention time preparative method: 9.0-10.2 min

Retention time analytical method: 2.72 min

[α]²⁰_{D}: +4.3° (c=1.00, MeOH)

Enantiomer 2:

(34.9 mg (21% yield)

Retention time preparative method: 10.4-11.6 min

Retention time analytical method: 3.18 min

[α]²⁰_{D}: −3.4° (c=1.00, MeOH)

### Intermediate 384 and Intermediate 385

### 5-bromo-3-[1-(1,4-dimethyl-1H-pyrazol-5-yl)ethoxy]pyridin-2-amine (Enantiomer 1 and Enantiomer 2)

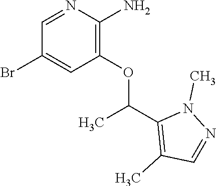 [SMILES: Cc1cnn(C)c1C(C)Oc1cc(Br)cnc1N]

The title compounds were prepared similarly to Intermediate 376 and Intermediate 377 starting from (rac)-5-bromo-3-[1-(1,4-dimethyl-1H-pyrazol-5-yl)ethoxy]-2-nitropyridine (452 mg, 1.32 mmol)

LC-MS (Method 1): Rₜ=0.97 min; MS (ESIneg): m/z=309 [M−H]⁻

¹H-NMR (400 MHz, CHLOROFORM-d): δ [ppm]=1.73 (d, 3H), 2.11 (s, 3H), 3.89 (s, 3H), 4.68 (br s, 2H), 5.38 (d, 1H), 6.77 (d, 1H), 7.25 (s, 1H), 7.70 (d, 1H).

Chiral Separation:

Preparative

Instrument: Sepiatec: Prep SFC100; Column: Chiralpak IG 5μ 250×30 mm; eluent A: CO2; eluent B: ethanol+0.2 vol % aqueous ammonia (32%); isocratic: 10% B; flow: 100 mL/min; temperature: 40° C.; BPR: 150 bar; UV: 254 nm;

Analytical

Instrument: Agilent: 1260, Aurora SFC-Modul; Column: Chiralpak IG 5μ 100×4.6 mm; eluent A: CO2; eluent B: ethanol+0.2 vol % aqueous ammonia (32%); isocratic: 10% B; flow: 4 mL/min; temperature: 37.5° C.; BPR: 100 bar; UV: 254 nm;

Enantiomer 1:

71.7 mg (32% yield)

Retention time preparative method: 11.00-14.50 min

Retention time analytical method: 3.55 min

[α]²⁰_{D}: +16.8° (c=1.00, MeOH)

Enantiomer 2:

64.8 mg (29%, yield)

Retention time preparative method: 14.50-19.50 min

Retention time analytical method: 4.52 min

[α]²⁰_{D}: −17.1° (c=1.00, MeOH)

### Intermediate 386

### 5-bromo-3-[(1R)-1-(3-fluoropyridin-2-yl)ethoxy]pyridin-2-amine

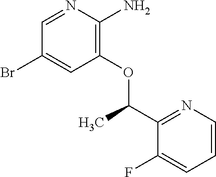 [SMILES: C[C@@H](Oc1cc(Br)cnc1N)c1ncccc1F]

To a suspension of 5-bromo-3-[(1R)-1-(3-fluoropyridin-2-yl)ethoxy]-2-nitropyridine (Intermediate 332, 370 mg, 65% purity, 703 μmol) in ethanol (3.8 mL) under argon were added iron (157 mg, 2.81 mmol) and acetic acid (1.9 mL). The mixture was stirred for 3 h at 80° C. The cold reaction mixture was diluted with dichloromethane, filtered and washed with dichloromethane. The filtrate was extracted twice with 2M NaOH-solution and filtered over celite. The organic layer was washed with brine, dried over sodium sulfate, filtered and concentrated under reduced pressure. The residue was purified by column chromatography (silica gel, hexane/ethyl acetate (0-90%) gradient) to give 197 mg (95% purity, 85% yield) of the title compound.

LC-MS (Method 1): Rₜ=1.01 min; MS (ESIpos): m/z=312 [M+H]⁺

¹H-NMR (400 MHz, CHLOROFORM-d): δ [ppm]=1.77 (d, 3H), 4.80 (br s, 2H), 5.63 (q, 1H), 7.02 (d, 1H), 7.29 (dt, 1H), 7.39-7.46 (m, 1H), 7.67 (d, 1H), 8.43 (dt, 1H).

### Intermediate 387

### 5-bromo-3-[(1R)-1-(5-fluoropyridin-2-yl)ethoxy]pyridin-2-amine

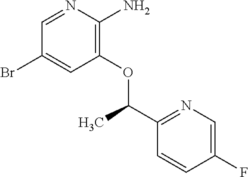 [SMILES: C[C@@H](Oc1cc(Br)cnc1N)c1ccc(F)cn1]

The compound was prepared similarly to Intermediate 386 starting from 5-bromo-3-[(1R)-1-(5-fluoropyridin-2-yl)ethoxy]-2-nitropyridine (Intermediate 331, 370 mg, 75% purity, 811 μmol) to give 200 mg (95% purity, 75% yield) of the title compound.

LC-MS (Method 1): Rₜ=1.04 min; MS (ESIpos): m/z=312 [M+H]⁺

¹H-NMR (400 MHz, CHLOROFORM-d): δ [ppm]=1.70 (d, 3H), 4.78 (br s, 2H), 5.36 (q, 1H), 6.86 (d, 1H), 7.31-7.46 (m, 2H), 7.68 (d, 1H), 8.45 (d, 1H).

### Intermediate 388

### 5-bromo-3-[(1S)-1-(2,6-difluorophenyl)ethoxy]pyridin-2-amine

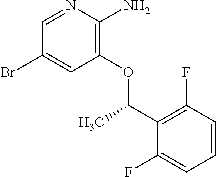 [SMILES: C[C@H](Oc1cc(Br)cnc1N)c1c(F)cccc1F]

To a suspension of 5-bromo-3-[(1S)-1-(2,6-difluorophenyl)ethoxy]-2-nitropyridine (Intermediate 330, 364 mg, 80% purity, 811 μmol) in ethanol (4.3 mL) under argon were added iron (181 mg, 3.24 mmol]) and acetic acid (2.2 mL). The mixture was stirred for 3 h at 80° C. The cold reaction mixture was diluted with dichloromethane, filtered and washed with dichloromethane. The filtrate was extracted twice with 2M NaOH-solution and filtered over celite. The organic layer was washed with brine, dried over sodium sulfate, filtered and concentrated under reduced pressure. The residue was purified by column chromatography (silica gel, hexane/ethyl acetate (0-50%) gradient) to give 233 mg (95% purity, 83% yield) of the title compound.

LC-MS (Method 1): Rₜ=1.23 min; MS (ESIpos): m/z=329 [M+H]⁺

¹H-NMR (400 MHz, CHLOROFORM-d) 5 [ppm]: 0.008 (0.50), 1.269 (0.51), 1.635 (0.51), 1.651 (0.46), 1.799 (15.80), 1.816 (16.00), 2.056 (0.71), 4.744 (2.92), 5.698 (1.00), 5.715 (3.06), 5.731 (3.02), 5.748 (0.96), 6.872 (0.45), 6.875 (0.64), 6.884 (4.85), 6.892 (0.62), 6.896 (0.94), 6.904 (9.74), 6.914 (0.75), 6.917 (0.67), 6.926 (5.49), 6.934 (0.65), 6.938 (0.44), 7.017 (5.91), 7.021 (6.01), 7.235 (1.18), 7.250 (2.39), 7.255 (1.71), 7.266 (1.96), 7.276 (1.49), 7.287 (1.63), 7.292 (2.09), 7.308 (1.03), 7.672 (8.09), 7.677 (8.36).

### Intermediate 389

### 5-bromo-3-[(1R)-1-(2,6-difluorophenyl)ethoxy]pyridin-2-amine

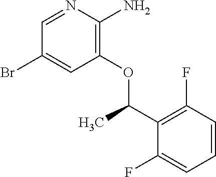 [SMILES: C[C@@H](Oc1cc(Br)cnc1N)c1c(F)cccc1F]

The compound was prepared similarly to Intermediate 386 starting from 5-bromo-3-[(1R)-1-(2,6-difluorophenyl)ethoxy]-2-nitropyridine (Intermediate 333, 322 mg, 80% purity, 717 μmol) to give 208 mg (88% yield) of the title compound.

LC-MS (Method 1): Rₜ=1.22 min; MS (ESIpos): m/z=329 [M+H]⁺

¹H-NMR (400 MHz, CHLOROFORM-d) 5 [ppm]: 1.269 (0.49), 1.799 (15.91), 1.815 (16.00), 2.055 (0.80), 4.744 (2.96), 5.698 (0.99), 5.715 (3.05), 5.731 (3.03), 5.748 (0.97), 6.875 (0.59), 6.884 (4.52), 6.892 (0.61), 6.896 (0.83), 6.904 (9.91), 6.913 (0.74), 6.917 (0.62), 6.925 (5.13), 6.934 (0.66), 6.938 (0.44), 7.017 (5.98), 7.021 (5.98), 7.234 (1.16), 7.250 (2.39), 7.255 (1.64), 7.266 (1.72), 7.276 (1.27), 7.287 (1.67), 7.292 (1.83), 7.308 (0.90), 7.672 (8.53), 7.677 (7.95).

### Intermediate 390

### (rac)-2-{1-[(2-amino-5-bromopyridin-3-yl)oxy]ethyl}benzonitrile

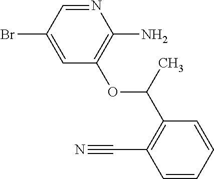 [SMILES: CC(Oc1cc(Br)cnc1N)c1ccccc1C#N]

(Rac)-2-{1-[(5-Bromo-2-nitropyridin-3-yl)oxy]ethyl}benzonitrile (Intermediate 334, 333 mg, 83% purity, 793 μmol) was dissolved in ethanol (2.3 mL). Acetic acid (1.6 mL) and iron (122 mg, 2.18 mmol) were added and the mixture was stirred for 2 h at 85° C. The reaction mixture was diluted with ethyl acetate, filtered through celite, basified with 2M sodium hydroxide solution and extracted 2 times with ethyl acetate. The combined organic layers were dried over a hydrophobic filter and concentrated. The residue was purified by column chromatography (silica gel, dichlormethane/methanol (0-5%) gradient) to give 142 mg (95% purity, 53% yield) of the title compound.

LC-MS (Method 1): Rₜ=1.10 min; MS (ESIpos): m/z=318 [M+H]⁺

¹H-NMR (400 MHz, DMSO-d6) δ[ppm]: 0.967 (0.52), 0.984 (1.18), 1.002 (0.57), 1.232 (1.14), 1.633 (2.36), 1.641 (15.85), 1.657 (16.00), 2.518 (2.20), 2.523 (1.42), 3.159 (0.52), 3.172 (0.42), 5.735 (0.98), 5.751 (3.59), 5.767 (3.56), 5.782 (0.97), 6.083 (6.74), 7.077 (6.13), 7.081 (6.22), 7.496 (1.96), 7.502 (2.12), 7.513 (2.36), 7.516 (2.66), 7.518 (2.77), 7.521 (2.43), 7.532 (2.27), 7.537 (2.62), 7.558 (10.37), 7.563 (10.02), 7.623 (0.41), 7.627 (0.42), 7.714 (1.15), 7.717 (1.25), 7.733 (3.62), 7.737 (3.92), 7.750 (8.08), 7.754 (8.19), 7.769 (1.23), 7.774 (0.52), 7.830 (1.09), 7.868 (3.66), 7.870 (4.03), 7.887 (2.90), 7.889 (3.99).

### Intermediate 391

### 3-{(1R)-1-[(2-amino-5-bromopyridin-3-yl)oxy]ethyl}benzonitrile

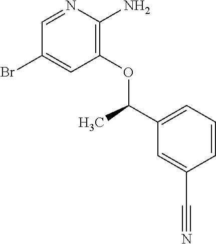 [SMILES: C[C@@H](Oc1cc(Br)cnc1N)c1cccc(C#N)c1]

To a suspension of 3-{(1R)-1-[(5-bromo-2-nitropyridin-3-yl)oxy]ethyl}benzonitrile (Intermediate 335, 371 mg, 50% purity, 533 μmol) in ethanol (2.8 mL) under argon were added iron (119 mg, 2.13 mmol) and acetic acid (1.4 mL). The mixture was stirred for 3 h at 80° C. The cold reaction mixture was diluted with dichloromethane, filtered and washed with dichloromethane. The filtrate was extracted twice with 2M NaOH-solution and filtered over celite. The organic layer was washed with brine, dried over sodium sulfate, filtered and concentrated under reduced pressure. The residue was purified by column chromatography (silica gel, hexane/ethyl acetate (0-90%) gradient) to give 135 mg (95% purity, 75% yield) of the title compound.

LC-MS (Method 1): Rₜ=1.10 min; MS (ESIpos): m/z=318 [M+H]⁺

¹H-NMR (400 MHz, CHLOROFORM-d) 5 [ppm]: 1.250 (2.23), 1.268 (4.18), 1.286 (1.96), 1.676 (16.00), 1.692 (15.62), 2.055 (8.07), 4.103 (0.55), 4.121 (1.62), 4.138 (1.52), 4.157 (0.53), 4.766 (2.60), 5.283 (0.77), 5.299 (2.44), 5.315 (2.39), 5.331 (0.75), 6.771 (4.42), 6.775 (4.41), 7.494 (1.24), 7.513 (3.54), 7.531 (2.89), 7.571 (1.45), 7.575 (3.02), 7.578 (2.03), 7.591 (0.96), 7.594 (1.75), 7.616 (1.83), 7.620 (3.54), 7.623 (1.95), 7.634 (0.96), 7.638 (2.93), 7.642 (4.30), 7.647 (4.00), 7.651 (1.73), 7.708 (6.10), 7.712 (5.73).

### Intermediate 392

### 4-{(1R)-1-[(2-amino-5-bromopyridin-3-yl)oxy]ethyl}benzonitrile

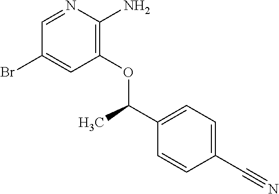 [SMILES: C[C@@H](Oc1cc(Br)cnc1N)c1ccc(C#N)cc1]

To a suspension of 4-{(1R)-1-[(5-bromo-2-nitropyridin-3-yl)oxy]ethyl}benzonitrile (Intermediate 336, 363 mg, 45% purity, 469 μmol) in ethanol (2.5 mL) under argon were added iron (105 mg, 1.88 mmol) and acetic acid (1.2 mL). The mixture was stirred for 3 h at 80° C. The cold reaction mixture was diluted with dichloromethane, filtered and washed with dichloromethane. The filtrate was extracted twice with 2M NaOH-solution and filtered over celite. The organic layer was washed with brine, dried over sodium sulfate, filtered and concentrated under reduced pressure. The residue was purified by column chromatography (silica gel, hexane/ethyl acetate (0-90%) gradient) to give 120 mg (95% purity, 76% yield) of the title compound.

LC-MS (Method 1): Rₜ=1.09 min; MS (ESIpos): m/z=318 [M+H]⁺

¹H-NMR (400 MHz, CHLOROFORM-d) 5 [ppm]: 1.251 (2.27), 1.268 (4.58), 1.286 (2.13), 1.676 (16.00), 1.693 (15.06), 2.055 (8.01), 4.104 (0.56), 4.121 (1.73), 4.139 (1.69), 4.157 (0.53), 4.760 (2.79), 5.297 (0.79), 5.314 (2.43), 5.330 (2.44), 5.345 (0.73), 6.748 (4.52), 6.753 (4.59), 7.447 (6.20), 7.450 (1.99), 7.464 (2.21), 7.468 (7.44), 7.676 (1.33), 7.680 (8.26), 7.684 (2.55), 7.696 (2.78), 7.700 (10.69), 7.705 (6.74).

### Intermediate 393

### 5-bromo-3-[(1R)-1-(3,5-difluoropyridin-4-yl)ethoxy]pyridin-2-amine

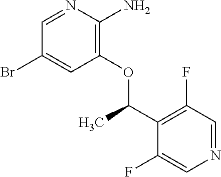 [SMILES: C[C@@H](Oc1cc(Br)cnc1N)c1c(F)cncc1F]

To a suspension of 5-bromo-3-[(1R)-1-(3,5-difluoropyridin-4-yl)ethoxy]-2-nitropyridine (Intermediate 337, 366 mg, 60% purity, 610 μmol) in ethanol (3.3 mL) under argon were added iron (136 mg, 2.44 mmol) and acetic acid (1.6 mL). The mixture was stirred for 3 h at 80° C. The cold reaction mixture was diluted with dichloromethane, filtered and washed with dichloromethane. The filtrate was extracted twice with 2M NaOH-solution and filtered over celite. The organic layer was washed with brine, dried over sodium sulfate, filtered and concentrated under reduced pressure. The residue was purified by column chromatography (silica gel, hexane/ethyl acetate (0-90%) gradient) to give 166 mg (95% purity, 78% yield) of the title compound.

LC-MS (Method 1): Rₜ=1.04 min; MS (ESIpos): m/z=330 [M+H]⁺

¹H-NMR (400 MHz, CHLOROFORM-d): δ [ppm]=1.81 (d, 3H), 4.74 (br s, 2H), 5.67-5.78 (m, 1H), 6.97 (d, 1H), 7.71 (d, 1H), 8.36 (s, 2H).

### Intermediate 394

### (rac)-5-bromo-3-(1-phenylpropoxy)pyridin-2-amine (Enantiomer 1 and Enantiomer 2)

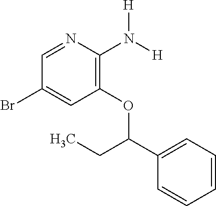 [SMILES: CCC(Oc1cc(Br)cnc1N)c1ccccc1]

(Rac)-5-bromo-2-nitro-3-(1-phenylpropoxy)pyridine (Intermediate 338, 324 mg, 962 μmol) was dissolved in ethanol (2.8 mL). Iron (148 mg, 2.64 mmol) and acetic acid (1.9 mL) were added and the mixture was stirred for 1 h at 85° C. The reaction mixture was tempered to rt, basified with 2M NaOH-solution, diluted with ethyl acetate and filtered over celite. The aqueous layer was extracted 2 times with ethyl acetate. The combined organic layers were washed with brine, dried over a hydrophobic filter and concentrated to give 320 mg (90% purity, 97% yield) of the title compound.

LC-MS (Method 1): Rₜ=1.28 min; MS (ESIpos): m/z=307 [M+H]⁺

¹H-NMR (400 MHz, DMSO-d6) δ[ppm]: 0.807 (0.44), 0.851 (0.61), 0.863 (1.19), 0.882 (2.72), 0.889 (6.62), 0.901 (2.12), 0.907 (16.00), 0.926 (7.02), 1.233 (1.62), 1.352 (0.68), 1.761 (0.80), 1.775 (1.07), 1.780 (0.98), 1.795 (1.72), 1.810 (1.28), 1.814 (1.33), 1.828 (1.07), 1.933 (1.34), 1.952 (2.19), 1.970 (1.77), 1.986 (1.84), 2.004 (1.01), 2.332 (1.18), 2.336 (0.52), 2.518 (5.77), 2.522 (3.76), 2.673 (1.19), 2.678 (0.54), 5.309 (1.78), 5.326 (2.49), 5.341 (1.74), 5.990 (0.44), 6.006 (0.43), 6.035 (6.70), 6.993 (5.79), 6.998 (5.80), 7.241 (0.73), 7.244 (1.37), 7.247 (0.86), 7.256 (1.01), 7.262 (3.85), 7.268 (1.32), 7.277 (1.82), 7.280 (3.42), 7.284 (1.76), 7.324 (3.99), 7.328 (1.98), 7.335 (0.80), 7.340 (4.07), 7.344 (8.11), 7.357 (1.94), 7.362 (4.67), 7.369 (1.28), 7.386 (0.51), 7.409 (7.12), 7.426 (4.78), 7.430 (3.36), 7.469 (8.92), 7.475 (8.56), 8.036 (1.10), 8.041 (1.46), 8.069 (0.60), 8.074 (0.48), 8.093 (0.57), 8.098 (0.53).

### Intermediate 395

### 3-{(1S)-1-[(2-amino-5-bromopyridin-3-yl)oxy]ethyl}benzonitrile

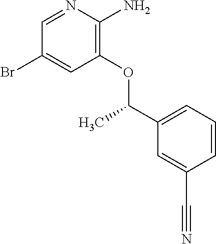 [SMILES: C[C@H](Oc1cc(Br)cnc1N)c1cccc(C#N)c1]

The compound was prepared similarly to Intermediate 393 starting from 3-{(1S)-1-[(5-bromo-2-nitropyridin-3-yl)oxy]ethyl}benzonitrile (Intermediate 339, 374 mg, 55% purity, 591 μmol) to give 167 mg (95% purity, 84% yield) of the title compound.

LC-MS (Method 1): Rₜ=1.09 min; MS (ESIneg): m/z=316 [M−H]⁻

¹H-NMR (400 MHz, CHLOROFORM-d) 5 [ppm]: 0.008 (0.44), 1.251 (4.15), 1.269 (9.14), 1.286 (4.37), 1.677 (15.50), 1.693 (16.00), 2.056 (15.38), 4.103 (1.01), 4.122 (2.90), 4.140 (3.03), 4.157 (1.01), 4.761 (2.53), 5.284 (0.75), 5.299 (2.35), 5.316 (2.35), 5.332 (0.73), 6.771 (4.27), 6.775 (4.36), 7.494 (1.18), 7.495 (1.18), 7.514 (3.38), 7.533 (2.88), 7.572 (1.38), 7.575 (2.97), 7.578 (2.04), 7.592 (0.94), 7.595 (1.71), 7.599 (0.99), 7.617 (1.80), 7.621 (3.45), 7.624 (2.03), 7.635 (0.94), 7.639 (3.03), 7.643 (4.79), 7.645 (3.19), 7.647 (3.95), 7.651 (1.75), 7.709 (5.80), 7.713 (6.32).

### Intermediate 396

### 4-{(1S)-1-[(2-amino-5-bromopyridin-3-yl)oxy]ethyl}benzonitrile

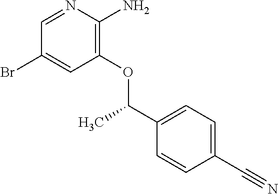 [SMILES: C[C@H](Oc1cc(Br)cnc1N)c1ccc(C#N)cc1]

The compound was prepared similarly to Intermediate 393 starting from 4-{(1S)-1-[(5-bromo-2-nitropyridin-3-yl)oxy]ethyl}benzonitrile (Intermediate 340, 366 mg, 50% purity, 526 μmol) to give 131 mg (95% purity, 74% yield) of the title compound.

LC-MS (Method 1): Rₜ=1.09 min; MS (ESIpos): m/z=318 [M+H]⁺

¹H-NMR (400 MHz, CHLOROFORM-d) 5 [ppm]: 0.007 (0.42), 1.250 (1.92), 1.268 (4.09), 1.286 (1.91), 1.676 (16.00), 1.692 (14.82), 2.055 (6.92), 4.103 (0.44), 4.121 (1.32), 4.139 (1.36), 4.157 (0.45), 4.759 (2.79), 5.297 (0.76), 5.313 (2.40), 5.329 (2.40), 5.345 (0.74), 6.747 (4.48), 6.751 (4.48), 7.447 (6.05), 7.450 (2.03), 7.463 (2.23), 7.467 (7.48), 7.675 (1.36), 7.680 (8.20), 7.684 (2.56), 7.696 (2.90), 7.699 (10.51), 7.704 (6.71).

### Intermediate 397

### 5-bromo-3-[(1R)-1-(2-fluorophenyl)ethoxy]pyridin-2-amine

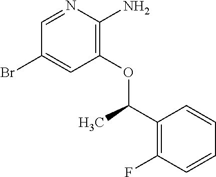 [SMILES: C[C@@H](Oc1cc(Br)cnc1N)c1ccccc1F]

5-Bromo-3-[(1R)-1-(2-fluorophenyl)ethoxy]-2-nitropyridine (Intermediate 344, 492 mg, 1.44 mmol) was dissolved in ethanol (7.7 mL). Iron (322 mg, 5.77 mmol) and acetic acid (3.8 mL) were added under argon and the mixture was stirred for 3 h at 80° C. The reaction mixture was allowed to cool down, diluted with dichloromethane and filtered. The filtrate was extracted with 2M NaOH. The organic phase was washed with brine, dried over sodium sulfate, filtered and concentrated to give 457 mg (85% purity, 87% yield) of the title compound.

LC-MS (Method 1): Rₜ=1.24 min; MS (ESIpos): m/z=311 [M+H]⁺

### Intermediate 398

### 5-bromo-3-[(1S)-1-(3,5-difluoropyridin-4-yl)ethoxy]pyridin-2-amine

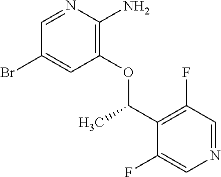 [SMILES: C[C@H](Oc1cc(Br)cnc1N)c1c(F)cncc1F]

The compound was prepared similarly to Intermediate 393 starting from 5-bromo-3-[(1S)-1-(3,5-difluoropyridin-4-yl)ethoxy]-2-nitropyridine (Intermediate 341, 366 mg, 68% purity, 691 μmol) to give 153 mg (95% purity, 64% yield) of the title compound.

LC-MS (Method 1): Rₜ=1.04 min; MS (ESIpos): m/z=330 [M+H]⁺

¹H-NMR (400 MHz, CHLOROFORM-d): δ [ppm]=1.81 (d, 3H), 4.74 (br s, 2H), 5.72 (d, 1H), 6.97 (d, 1H), 7.71 (d, 1H), 8.36 (s, 2H).

### Intermediate 399

### (rac)-5-bromo-3-[1-(1-methyl-1H-pyrazol-5-yl)ethoxy]pyridin-2-amine

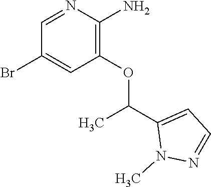 [SMILES: CC(Oc1cc(Br)cnc1N)c1ccnn1C]

To a suspension of (rac)-5-bromo-3-[1-(1-methyl-1H-pyrazol-5-yl)ethoxy]-2-nitropyridine (Intermediate 343, 375 mg, 1.15 mmol) in ethanol (6.1 mL) under argon were added iron (256 mg, 4.59 mmol) and acetic acid (3.1 mL) and the mixture was stirred for 3 h at 80° C. The reaction mixture was allowed to cool down, diluted with dichloromethane and filtered. The filtrate was extracted with 2M NaOH and filtered over celite. The organic layer was washed with brine, dried over sodium sulfate, filtered and concentrated to give 318 mg (96% purity, 90% yield) of the title compound.

LC-MS (Method 1): Rₜ=0.90 min; MS (ESIneg): m/z=295 [M−H]⁻

¹H-NMR (400 MHz, CHLOROFORM-d): δ [ppm]=1.75 (d, 3H), 3.85 (s, 3H), 4.66 (br s, 2H), 5.40 (q, 1H), 6.30 (d, 1H), 7.00 (d, 1H), 7.45 (d, 1H), 7.74 (d, 1H).

### Intermediate 400

### 5-bromo-N³-methyl-N³-[(1R)-1-phenylethyl]pyridine-2,3-diamine

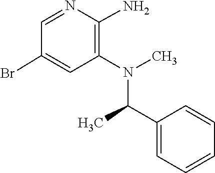 [SMILES: C[C@H](c1ccccc1)N(C)c1cc(Br)cnc1N]

5-Bromo-N-methyl-2-nitro-N-[(1R)-1-phenylethyl]pyridin-3-amine (Intermediate 345, 182 mg, 541 μmol) was suspended in ethanol (2.9 mL). Iron (121 mg, 2.17 mmol) and acetic acid (1.4 mL) were added and the mixture was stirred for 3 h at 80° C. The cold reaction mixture was diluted with dichloromethane and filtered. The filtrate was extracted with 2M NaOH and filtered over celite. The organic phase was washed with brine, dried over sodium sulfate, filtered and concentrated to give 103 mg (90% purity, 56% yield) of the title compound.

LC-MS (Method 1): Rₜ=1.31 min; MS (ESIpos): m/z=306 [M+H]⁺

¹H-NMR (400 MHz, CHLOROFORM-d): δ [ppm]=1.38 (br d, 3H), 2.43 (s, 3H), 4.25 (br q, 1H), 4.88 (br s, 2H), 7.17 (s, 1H), 7.27-7.40 (m, 5H), 7.87 (s, 1H).

### Intermediate 401

### 5-bromo-3-[(1R)-1-(4-methylpyridin-2-yl)ethoxy]pyridin-2-amine

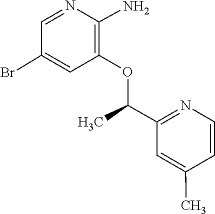 [SMILES: Cc1ccnc([C@@H](C)Oc2cc(Br)cnc2N)c1]

5-Bromo-3-[(1R)-1-(4-methylpyridin-2-yl)ethoxy]-2-nitropyridine (Intermediate 346, 187 mg, 553 μmol) was suspended in ethanol (3.0 mL). Iron (124 mg, 2.21 mmol) and acetic acid (1.5 mL) were added and the mixture was stirred for 3 h at 80° C. The cold reaction mixture was diluted with dichloromethane and filtered. The filtrate was extracted with 2M NaOH and filtered over celite. The organic phase was washed with brine, dried over sodium sulfate, filtered and concentrated. The residue was purified by flash column chromatography (silica gel, hexane/ethyla acetate (0-100%) gradient) to give 185 mg (90% purity, 98% yield) of the title compound.

LC-MS (Method 1): Rₜ=1.03 min; MS (ESIpos): m/z=308 [M+H]⁺

¹H NMR (400 MHz, CHLOROFORM-d, 22° C.): δ=8.44 (d, 1H), 7.67 (d, 1H), 7.13 (s, 1H), 7.04 (dd, 1H), 6.88 (d, 1H), 5.30 (q, 1H), 4.80 (br s, 2H), 2.33 (s, 3H), 1.69 ppm (d, 3H).

### Intermediate 402

### 5-bromo-3-[(1R)-1-(6-methylpyridin-2-yl)ethoxy]pyridin-2-amine

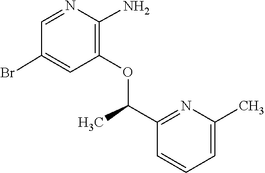 [SMILES: Cc1cccc([C@@H](C)Oc2cc(Br)cnc2N)n1]

To a suspension of 5-bromo-3-[(1R)-1-(6-methylpyridin-2-yl)ethoxy]-2-nitropyridine (Intermediate 347, 156 mg, 461 μmol) in ethanol (2.5 mL) were added iron (103 mg, 1.85 mmol) and acetic acid (1.2 mL). The mixture was stirred for 3 h at 80° C. The cold reaction mixture was diluted with dichloromethane and filtered. The filtrate was extracted with 2M NaOH and filtered over celite. The organic phase was washed with brine, dried over sodium sulfate, filtered and concentrated. The residue was purified by flash column chromatography (silica gel, hexane/ethyla acetate (0-100%) gradient) to give 123 mg (98% purity, 85% yield) of the title compound.

¹H NMR (400 MHz, CHLOROFORM-d, 22° C.): δ=7.66 (d, 1H), 7.56 (t, 1H), 7.09 (dd, 2H), 6.90 (d, 1H), 5.30 (q, 1H), 4.81 (br s, 2H), 2.58 (s, 3H), 1.70 ppm (d, 3H).

### Intermediate 403

### 5-bromo-3-[(1R)-1-(3-chlorophenyl)ethoxy]pyridin-2-amine

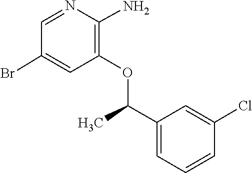 [SMILES: C[C@@H](Oc1cc(Br)cnc1N)c1cccc(Cl)c1]

5-Bromo-3-[(1R)-1-(3-chlorophenyl)ethoxy]-2-nitropyridine (495 mg, 98% purity, 1.36 mmol) was dissolved in ethanol (20 mL). Acetic acid (3.6 mL) was added dropwise. Iron (303 mg, 5.43 mmol) was added and the mixture was stirred for 30 min at 80° C. The reaction mixture was allowed to reach rt, diluted with dichloromethane and filtered over celite. The organic phase was washed with 2M hydrochloric acid. The aqueous phase was extracted with dichloromethane. The combined organic layers were washed with saturated sodium bicarbonate solution, dried over a hydrophobic filter and concentrated. The residue was purified by column chromatography (silica gel, hexane/ethyl acetate (0-100%) gradient) to give 380 mg (98% purity, 84% yield) of the title compound.

LC-MS (Method 1): Rₜ=1.29 min; MS (ESIpos): m/z=327 [M+H]⁺

### Intermediate 404

### 5-bromo-3-[(1R)-1-(2-methoxyphenyl)ethoxy]pyridin-2-amine

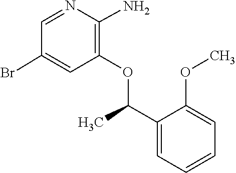 [SMILES: COc1ccccc1[C@@H](C)Oc1cc(Br)cnc1N]

5-Bromo-3-[(1R)-1-(2-methoxyphenyl)ethoxy]-2-nitropyridine (470 mg, 97% purity, 1.29 mmol) was dissolved in ethanol (10 mL). Acetic acid (3.4 mL) was added dropwise. Iron (288 mg, 5.16 mmol) was added and the mixture was stirred for 30 min at 80° C. The reaction mixture was allowed to cool down, diluted with dichloromethane and filtered over celite. The organic phase was washed with 2M hydrochloric acid. The aqueous phase was extracted with dichloromethane. The combined organic layers were washed with saturated sodium bicarbonate solution, dried over a hydrophobic filter and concentrated. The residue was purified by column chromatography (silica gel, hexane/ethyl acetate (0-40%) gradient) to give 391 mg (100% purity, 94% yield) of the title compound.

[α]²⁰_{D}: −65.9° (c=1.00, methanol)

LC-MS (Method 2): Rₜ=1.17 min; MS (ESIpos): m/z=323 [M+H]⁺

¹H-NMR (400 MHz, DMSO-d6) δ[ppm]: 1.533 (5.88), 1.548 (5.88), 2.518 (1.02), 2.523 (0.65), 3.882 (16.00), 5.670 (1.26), 5.686 (1.26), 6.020 (2.76), 6.797 (2.37), 6.803 (2.39), 6.912 (0.70), 6.914 (0.74), 6.930 (1.53), 6.932 (1.56), 6.949 (0.86), 6.951 (0.86), 7.034 (1.37), 7.036 (1.39), 7.055 (1.74), 7.056 (1.60), 7.245 (0.86), 7.248 (0.98), 7.263 (0.97), 7.265 (1.04), 7.267 (1.11), 7.269 (1.00), 7.283 (0.68), 7.287 (0.70), 7.342 (1.41), 7.346 (1.33), 7.361 (1.32), 7.365 (1.18), 7.481 (3.34), 7.486 (3.32).

### Intermediate 405

### 5-bromo-3-[(1R)-1-(pyridin-3-yl)ethoxy]pyridin-2-amine

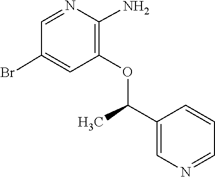 [SMILES: C[C@@H](Oc1cc(Br)cnc1N)c1cccnc1]

5-Bromo-2-nitro-3-[(1R)-1-(pyridin-3-yl)ethoxy]pyridine (203 mg, 82% purity, 512 μmol) was dissolved in ethanol (2.7 mL). Iron (114 mg, 2.05 mmol) and acetic acid (1.3 mL) were added and the mixture was stirred for 3 h at 80° C. The cold reaction mixture was diluted with dichloromethane and filtered. The filtrate was extracted with 2 M sodium hydroxide solution. The organic phase was washed with water and brine, dried and concentrated. The residue was purified by column chromatography (silica gel, hexane/ethyl acetate (0-100%) gradient) to give 88.3 mg (89% purity, 52% yield) of the title compound.

LC-MS (Method 1): Rₜ=0.90 min; MS (ESIneg): m/z=292 [M−H]⁻

¹H-NMR (400 MHz, DMSO-d6) δ[ppm]: 1.570 (16.00), 1.586 (15.99), 2.518 (3.41), 2.523 (2.20), 5.658 (1.02), 5.673 (3.48), 5.689 (3.45), 5.705 (1.01), 5.758 (6.68), 6.084 (8.13), 7.194 (6.55), 7.199 (6.76), 7.369 (2.60), 7.381 (2.75), 7.388 (2.85), 7.400 (2.86), 7.515 (8.98), 7.520 (8.71), 7.881 (2.07), 7.886 (3.37), 7.891 (2.23), 7.901 (2.01), 7.905 (3.07), 7.910 (1.98), 8.480 (4.54), 8.484 (4.51), 8.492 (4.57), 8.496 (4.28), 8.687 (5.36), 8.692 (5.37).

### Intermediate 406

### 5-bromo-3-[(1R)-1-(1,3-thiazol-2-yl)ethoxy]pyridin-2-amine

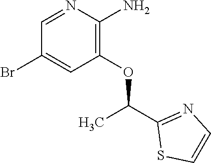 [SMILES: C[C@@H](Oc1cc(Br)cnc1N)c1nccs1]

5-Bromo-2-nitro-3-[(1R)-1-(1,3-thiazol-2-yl)ethoxy]pyridine (200 mg, 606 μmol) was dissolved in ethanol (3.2 mL). Acetic acid (1.6 mL) and iron (135 mg, 2.42 mmol) were added and the mixture was stirred for 1.5 h at 80° C. The reaction mixture was allowed to cool down, diluted with ethyl acetate and filtered. The filtrate was concentrated and diluted with ethyl acetate again. The organic phase was washed with saturated sodium bicarbonate solution and brine, dried over a hydrophobic filter and concentrated to give 157 mg (97% purity, 84% yield) of the title compound.

[α]²⁰_{D}: +26.3° (c=1.00, methanol)

LC-MS (Method 1): Rₜ=0.95 min; MS (ESIpos): m/z=300 [M+H]⁺

¹H-NMR (400 MHz, DMSO-d₆): δ [ppm]=1.70 (d, 3H), 5.90 (q, 1H), 6.02 (s, 2H), 7.28 (d, 1H), 7.59 (d, 1H), 7.74 (d, 1H), 7.81 (d, 1H).

### Intermediate 407

### 5-bromo-3-[(1R)-1-(3-fluorophenyl)ethoxy]pyridin-2-amine

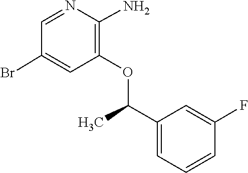 [SMILES: C[C@@H](Oc1cc(Br)cnc1N)c1cccc(F)c1]

5-Bromo-3-[(1R)-1-(3-fluorophenyl)ethoxy]-2-nitropyridine (343 mg, 100% purity, 1.01 mmol) was dissolved in ethanol (12 mL). Acetic acid (2.7 mL) and iron (225 mg, 4.02 mmol) were added and the mixture was stirred for 30 min at 80° C. The reaction mixture was allowed to cool down, diluted with dichloromethane and filtered over celite. The organic phase was washed with 2M hydrochloric acid. The aqueous phase was extracted with dichloromethane. The combined organic layers were washed with saturated sodium bicarbonate solution, dried over a hydrophobic filter and concentrated. The residue was purified by column chromatography (silica gel, hexane/ethyl acetate (0-80%) gradient) to give 270 mg (97% purity, 84% yield) of the title compound.

[α]²⁰_{D}: +17.3° (c=1.00, methanol)

LC-MS (Method 1): Rₜ=1.21 min; MS (ESIpos): m/z=311 [M+H]⁺

¹H-NMR (400 MHz, DMSO-d6) δ[ppm]: 1.153 (1.82), 1.171 (3.65), 1.189 (1.76), 1.535 (15.95), 1.551 (16.00), 1.986 (6.83), 2.518 (1.65), 2.523 (0.99), 3.998 (0.50), 4.016 (1.48), 4.034 (1.43), 4.052 (0.46), 5.592 (0.97), 5.608 (3.27), 5.623 (3.24), 5.639 (0.95), 6.084 (8.35), 7.069 (1.09), 7.072 (1.20), 7.076 (1.27), 7.079 (1.26), 7.092 (2.41), 7.097 (2.18), 7.099 (2.60), 7.119 (8.15), 7.124 (7.32), 7.284 (3.00), 7.303 (4.42), 7.328 (2.12), 7.335 (2.39), 7.338 (1.83), 7.354 (2.10), 7.360 (2.35), 7.364 (2.09), 7.367 (2.58), 7.382 (2.50), 7.387 (3.16), 7.402 (3.07), 7.407 (1.68), 7.422 (1.44), 7.506 (9.38), 7.511 (9.00).

### Intermediate 408

### (rac)-5-bromo-3-[(1-(1,3-oxazol-4-yl)ethoxy]pyridin-2-amine

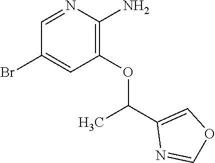 [SMILES: CC(Oc1cc(Br)cnc1N)c1cocn1]

5-Bromo-2-nitro-3-[1-(1,3-oxazol-4-yl)ethoxy]pyridine (185 mg, 589 μmol) was dissolved in ethanol (3.1 mL). Acetic acid (1.6 mL) and iron (98.7 mg, 1.77 mmol) were added and the mixture was stirred for 1.5 h at 80° C. The reaction mixture was allowed to cool down, diluted with ethyl acetate and filtered. The filtrate was concentrated and diluted with ethyl acetate again. The organic phase was washed with saturated sodium bicarbonate solution and brine, dried over a hydrophobic filter and concentrated to give 140 mg (100% purity, 84% yield) of the title compound.

LC-MS (Method 2): Rₜ=0.71 min; MS (ESIpos): m/z=284 [M+H]⁺

¹H-NMR (400 MHz, DMSO-d₆): δ [ppm]=1.57 (br d, 3H), 5.46-5.54 (m, 1H), 5.91 (br s, 2H), 7.34 (br s, 1H), 7.58 (br s, 1H), 8.21 (br s, 1H), 8.38 (br s, 1H).

### Intermediate 409

### (rac)-5-bromo-3-[1-(1,3-oxazol-2-yl)ethoxy]pyridin-2-amine

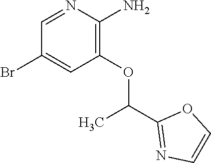 [SMILES: CC(Oc1cc(Br)cnc1N)c1ncco1]

(Rac)-5-bromo-2-nitro-3-[1-(1,3-oxazol-2-yl)ethoxy]pyridine (155 mg, 493 μmol) was dissolved in ethanol (2.6 mL). Acetic acid (1.3 mL) and iron (82.7 mg, 1.48 mmol) were added and the mixture was stirred for 2 h at 80° C. The reaction mixture was allowed to cool down, diluted with ethyl acetate and filtered. The filtrate was concentrated and diluted with ethyl acetate again. The organic phase was washed with saturated sodium bicarbonate solution and brine, dried over a hydrophobic filter and concentrated to give 107 mg (76% purity, 58% yield) of the title compound.

LC-MS (Method 1): Rₜ=0.89 min; MS (ESIpos): m/z=284 [M+H]⁺

¹H-NMR (400 MHz, DMSO-d₆): δ [ppm]=1.68 (d, 3H), 5.69 (q, 1H), 5.96 (s, 2H), 7.25 (s, 1H), 7.31 (d, 1H), 7.60 (d, 1H), 8.15 (s, 1H).

### Intermediate 410

### 5-bromo-3-[(1R)-1-(pyrimidin-2-yl)ethoxy]pyridin-2-amine

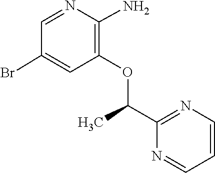 [SMILES: C[C@@H](Oc1cc(Br)cnc1N)c1ncccn1]

2-{(1R)-1-[(5-bromo-2-nitropyridin-3-yl)oxy]ethyl}pyrimidine (679 mg, 2.09 mmol) was dissolved in ethanol (18 mL) under argon. Acetic acid (5.6 mL) and iron (700 mg, 12.5 mmol) were added and the mixture was stirred for 4 h at 80° C. The reaction mixture was allowed to cool down, diluted with ethyl acetate and filtered. The filtrate was concentrated and diluted with ethyl acetate again. The organic phase was washed with saturated sodium bicarbonate solution, dried and concentrated. The residue was purified by column chromatography (silica gel, dichloromethane/ethanol (0-10%) gradient) to give 600 mg (97% yield) of the title compound.

LC-MS (Method 1): Rₜ=0.85 min; MS (ESIpos): m/z=295 [M+H]⁺

¹H NMR (DMSO-de, 400 MHz): δ (ppm) 8.84 (d, 2H), 7.53 (d, 1H), 7.46 (t, 1H), 6.99 (d, 1H), 5.96 (s, 2H), 5.52 (q, 1H), 1.67 (d, 3H).

### Intermediate 411

### 5-bromo-3-[(1R)-1-(pyridin-3-yl)ethoxy]pyridin-2-amine

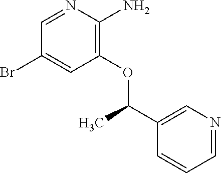 [SMILES: C[C@@H](Oc1cc(Br)cnc1N)c1cccnc1]

5-Bromo-2-nitro-3-[(1R)-1-(pyridin-3-yl)ethoxy]pyridine (4.95 g, 33% purity, 5.07 mmol) was dissolved in acetic acid (9.9 mL) and methanol (9.9 mL). Iron (849 mg, 15 mmol) was added and the mixture was stirred for 1.5 h at 85° C. The reaction mixture was allowed to cool down, diluted with ethyl acetate and filtered. The filtrate was concentrated and diluted with dichloromethane. The organic phase was washed with saturated sodium bicarbonate solution and brine, dried over a hydrophobic filter and concentrated. The residue was purified by column chromatography (silica gel, hexane/ethanol (0-30%) gradient) to give 950 mg (63% yield) of the title compound.

LC-MS (Method 1): Rₜ=0.89 min; MS (ESIpos): m/z=294 [M+H]⁺

¹H-NMR (400 MHz, DMSO-d₆): δ [ppm]=1.58 (d, 3H), 5.68 (q, 1H), 6.09 (s, 2H), 7.20 (d, 1H), 7.38 (dd, 1H), 7.52 (d, 1H), 7.90 (dt, 1H), 8.49 (dd, 1H), 8.69 (d, 1H).

### Intermediate 412

### 5-bromo-3-[(1R)-1-(1,2-thiazol-5-yl)ethoxy]pyridin-2-amine

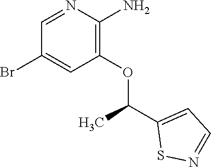 [SMILES: C[C@@H](Oc1cc(Br)cnc1N)c1ccns1]

5-Bromo-2-nitro-3-[(1R)-1-(1,2-thiazol-5-yl)ethoxy]pyridine (157 mg, 476 μmol) was dissolved in ethanol (2.5 mL). Acetic acid (1.3 mL) and iron (79.7 mg, 1.43 mmol) were added and the mixture was stirred for 1.5 h at 80° C. The reaction mixture was allowed to cool down, diluted with ethyl acetate and filtered. The filtrate was concentrated and diluted with ethyl acetate again. The organic phase was washed with saturated sodium bicarbonate solution and brine, dried over a hydrophobic filter and concentrated to give 125 mg (100% purity, 88% yield) of the title compound.

LC-MS (Method 1): Rₜ=0.97 min; MS (ESIpos): m/z=300 [M+H]⁺

¹H-NMR (400 MHz, DMSO-d₆): δ [ppm]=1.67 (d, 3H), 6.03 (s, 2H), 6.09 (q, 1H), 7.31 (d, 1H), 7.47 (dd, 1H), 7.58 (d, 1H), 8.50 (d, 1H).

### Intermediate 413

### (rac)-5-bromo-3-[1-(2,6-dichlorophenyl)ethoxy]pyridin-2-amine

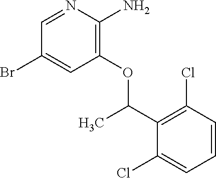 [SMILES: CC(Oc1cc(Br)cnc1N)c1c(Cl)cccc1Cl]

(Rac)-5-Bromo-3-[1-(2,6-dichlorophenyl)ethoxy]-2-nitropyridine (977 mg, 2.49 mmol) was dissolved in methanol (13 mL) and acetic acid (13 mL). Iron (418 mg, 7.48 mmol) was added and the mixture was stirred for 1 h at 85° C. The reaction mixture was allowed to cool down, filtered and the filtrate was concentrated. The residue was purified by column chromatography (silica gel, hexane/ethyl acetate (0-70%) gradient) to give 950 mg (63% yield) of the title compound.

LC-MS (Method 1): Rₜ=1.39 min; MS (ESIpos): m/z=361 [M+H]⁺

¹H NMR (DMSO-de, 400 MHz): δ (ppm) 7.53 (d, 1H), 7.47-7.51 (m, 2H), 7.33-7.39 (m, 1H), 6.73 (d, 1H), 5.94-6.03 (m, 3H), 1.76 (d, 3H).

### Intermediate 414

### (rac)-4-(3-{1-[(2-amino-5-bromopyridin-3-yl)oxy]ethyl}phenyl)-2-methylbut-3-yn-2-ol

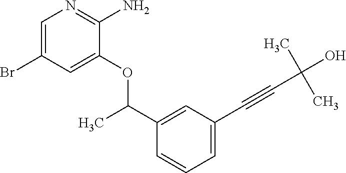 [SMILES: CC(Oc1cc(Br)cnc1N)c1cccc(C#CC(C)(C)O)c1]

5-Bromo-3-{[1-(3-{3-methyl-3-[(-oxan-2-yl)oxy]but-1-yn-1-yl}phenyl)ethyl]oxy}-2-nitropyridine (214 mg, 437 μmol) was suspended in ethanol (2.3 mL). Acetic acid (1.2 mL) and iron (97.7 mg, 1.75 mmol) were added and the mixture was stirred for 3 h at 80° C. and over night at room temperature. The reaction mixture was heated to 80° C., after 3 h hydrochloric acid in 1,4-dioxane (30 μL, 4M) was added and stirred for 3 h at 80° C. again. The reaction mixture was concentrated and diluted with dichloromethane/ethanol 9:1 and filtered. The filtrate was dried and concentrated. The residue was purified by column chromatography (silica gel, dichloromethane/methanol (0-5%) gradient) to give 161 mg (98% yield) of the title compound.

LC-MS (Method 1): Rₜ=1.16 min; MS (ESIpos): m/z=375 [M+H]⁺

¹H NMR (DMSO-d₆, 400 MHz): δ (ppm) 7.50 (d, 1H), 7.43-7.47 (m, 2H), 7.32-7.37 (m, 1H), 7.25-7.30 (m, 1H), 7.05 (d, 1H), 6.09 (s, 2H), 5.58 (q, 1H), 5.47 (s, 1H), 1.54 (d, 3H), 1.45 (s, 6H).

### Intermediate 415

### 5-bromo-3-[(6,7-dihydro-5H-cyclopenta[b]pyridin-7-yl)oxy]pyridin-2-amine (Enantiomer 1 and Enantiomer 2)

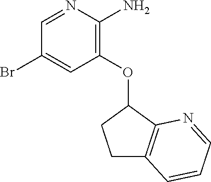 [SMILES: Nc1ncc(Br)cc1OC1CCc2cccnc21]

(Rac)-7-[(5-Bromo-2-nitropyridin-3-yl)oxy]-6,7-dihydro-5H-cyclopenta[b]pyridine (935 mg, 2.78 mmol) was suspended in ethanol (15 mL). Iron (621 mg, 11.1 mmol) and acetic acid (7.4 mL) were added and the mixture was stirred for 3 h at 80° C. The reaction mixture was allowed to cool down, diluted with dichloromethane and filtered. The filtrate was concentrated, diluted with saturated sodium bicarbonate solution and ethyl acetate/methanol 95:5 and filtered over celite. The layers were separated. The aqueous phase was extracted with ethyl acetate/methanol 95:5. The combined organic layers were dried and concentrated. The crude product was purified by column chromatography (silica gel, dichloromethane/ethanol (0-5%) gradient) to give 879 mg (103% yield) of the title compound.

LC-MS (Method 1): Rₜ=1.03 min; MS (ESIpos): m/z=306 [M+H]⁺

¹H NMR (DMSO-de, 400 MHz): δ (ppm) 8.43-8.47 (m, 1H), 7.78 (dd, 1H), 7.68 (d, 1H), 7.61 (d, 1H), 7.32 (dd, 1H), 5.89 (s, 2H), 5.73 (dd, 1H), 3.08-3.18 (m, 1H), 2.89 (ddd, 1H), 2.52-2.60 (m, 1H), 2.21 (dddd, 1H).

### Intermediate 416 and Intermediate 417

### 5-bromo-3-[(6,7-dihydro-5H-cyclopenta[b]pyridin-7-yl)oxy]pyridin-2-amine (Enantiomer 1 and Enantiomer 2)

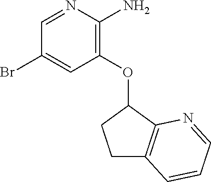 [SMILES: Nc1ncc(Br)cc1OC1CCc2cccnc21]

The compound from Intermediate 415 was separated into enantiomers by preparative chiral HPLC.

Preparative

Instrument: PrepCon Labomatic HPLC-4; Column: Reprosil NR 8μ, 250×50; eluent A: hexane+0.1 vol % diethylamine; eluent B: ethanol; isocratic: 40% B; flow: 100 mL/min; temperature: 25° C.; UV: 325 nm

Analytical

Instrument: Thermo Fisher UltiMate 3000; Column: Reprosil NR 5μ, 100×4.6; eluent A: hexane+0.1 vol % diethylamine; eluent B: ethanol; isocratic: 40% B; flow: 1.4 mL/min; temperature: 25° C.; UV: 325 nm

Enantiomer 1:

127.9 mg (27% yield)

Retention time preparative method: 8.89-11.57 min

Retention time analytical method: 2.55 min

LC-MS (Method 1): Rₜ=1.00 min; MS (ESIpos): m/z=306 [M+H]⁺

¹H-NMR (400 MHz, CHLOROFORM-d): δ [ppm]=2.30 (dddd, 1H), 2.62 (dddd, 1H), 2.95 (ddd, 1H), 3.11-3.23 (m, 1H), 4.89 (br s, 2H), 5.56 (dd, 1H), 7.21-7.26 (m, 1H), 7.46 (d, 1H), 7.65 (dd, 1H), 7.77 (d, 1H), 8.45-8.59 (m, 1H).

Enantiomer 2:

123.1 mg (26% yield)

Retention time preparative method: 14.52-18.00 min

Retention time analytical method: 5.27 min

LC-MS (Method 1): Rₜ=1.00 min; MS (ESIpos): m/z=306 [M+H]⁺

¹H-NMR (400 MHz, CHLOROFORM-d): δ [ppm]=2.25-2.35 (m, 1H), 2.57-2.68 (m, 1H), 2.97 (br dd, 1H), 3.12-3.23 (m, 1H), 4.76-5.04 (m, 2H), 5.54-5.59 (m, 1H), 7.22-7.26 (m, 1H), 7.46 (d, 1H), 7.65 (dd, 1H), 7.77 (d, 1H), 8.46-8.58 (m, 1H).

### Intermediate 418

### (rac)-5-bromo-3-[1-(4,5-dimethyl-1,3-thiazol-2-yl)ethoxy]pyridin-2-amine

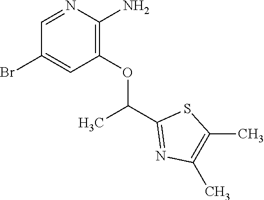 [SMILES: Cc1nc(C(C)Oc2cc(Br)cnc2N)sc1C]

(Rac)-5-Bromo-3-[1-(4,5-dimethyl-1,3-thiazol-2-yl)ethoxy]-2-nitropyridine (269 mg, 751 μmol) was dissolved in ethanol (4 mL) under argon. Acetic acid (2 mL) and iron (126 mg, 2.25 mmol) were added and the mixture was stirred for 1.5 h at 80° C. The reaction mixture was allowed to cool down, diluted with ethyl acetate and filtered. The filtrate was concentrated and diluted in ethyl acetate. The organic phase was washed with saturated sodium bicarbonate solution and brine, dried over a hydrophobic filter and concentrated to give 194 mg (96% purity, 76% yield) of the title compound.

LC-MS (Method 1): Rₜ=1.13 min; MS (ESIpos): m/z=328 [M+H]⁺

¹H-NMR (400 MHz, DMSO-d₆): δ [ppm]=1.64 (d, 3H), 2.24 (s, 3H), 2.29 (s, 3H), 5.70-5.77 (m, 1H), 5.99 (s, 2H), 7.26 (d, 1H), 7.58 (d, 1H).

### Intermediate 419

### (rac)-5-bromo-3-[1-(5-methyl-1,3-thiazol-2-yl)ethoxy]pyridin-2-amine

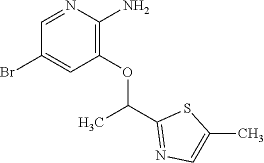 [SMILES: Cc1cnc(C(C)Oc2cc(Br)cnc2N)s1]

(Rac)-5-Bromo-3-[1-(5-methyl-1,3-thiazol-2-yl)ethoxy]-2-nitropyridine (257 mg, 747 μmol) was dissolved in ethanol (4 mL) under argon. Acetic acid (2 mL) and iron (125 mg, 2.24 mmol) were added and the mixture was stirred for 1.5 h at 80° C. The reaction mixture was allowed to reach rt, diluted with ethyl acetate and filtered. The filtrate was concentrated and diluted in ethyl acetate again. The organic phase was washed with saturated sodium bicarbonate solution and brine, dried over a hydrophobic filter and concentrated to give 212 mg (96% purity, 87% yield) of the title compound.

LC-MS (Method 1): Rₜ=1.06 min; MS (ESIpos): m/z=314 [M+H]⁺

¹H-NMR (400 MHz, DMSO-d₆): δ [ppm]=1.66 (d, 3H), 2.41 (d, 3H), 5.79 (q, 1H), 6.00 (s, 2H), 7.26 (d, 1H), 7.46 (d, 1H), 7.58 (d, 1H).

### Intermediate 420

### (rac)-5-bromo-3-[1-(1,3-thiazol-4-yl)ethoxy]pyridin-2-amine

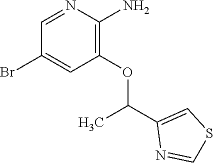 [SMILES: CC(Oc1cc(Br)cnc1N)c1cscn1]

(Rac)-5-Bromo-2-nitro-3-[1-(1,3-thiazol-4-yl)ethoxy]pyridine (237 mg, 718 μmol) was dissolved in ethanol (3.8 mL). Acetic acid (1.9 mL) and iron (120 mg, 2.15 mmol) were added and the mixture was stirred for 1.5 h at 80° C. The reaction mixture was allowed to cool down, diluted with ethyl acetate and filtered. The filtrate was concentrated and diluted in ethyl acetate again. The organic phase was washed with saturated sodium bicarbonate solution and brine, dried over a hydrophobic filter and concentrated to give 185 mg (100% purity, 86% yield) of the title compound.

LC-MS (Method 1): Rₜ=0.96 min; MS (ESIpos): m/z=300 [M+H]⁺

¹H-NMR (400 MHz, DMSO-d₆): δ [ppm]=1.63 (d, 3H), 5.70 (q, 1H), 5.96 (s, 2H), 7.25 (d, 1H), 7.56 (d, 1H), 7.78 (d, 1H), 9.10 (d, 1H).

### Intermediate 421

### 5-bromo-3-[(1R)-1-(5-methyl-1,3,4-oxadiazol-2-yl)ethoxy]pyridin-2-amine

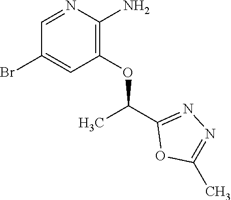 [SMILES: Cc1nnc([C@@H](C)Oc2cc(Br)cnc2N)o1]

5-Bromo-3-[(1R)-1-(5-methyl-1,3,4-oxadiazol-2-yl)ethoxy]-2-nitropyridine (160 mg, 486 μmol) was dissolved in ethanol (2.6 mL). Acetic acid (1.3 mL) and iron (81.4 mg, 1.46 mmol) were added and the mixture was stirred for 1.5 h at 80° C. The reaction mixture was allowed to cool down, diluted with ethyl acetate and filtered. The filtrate was concentrated and diluted in ethyl acetate again. The organic phase was washed with saturated sodium bicarbonate solution and brine, dried over a hydrophobic filter and concentrated to give 101 mg (93% purity, 65% yield) of the title compound.

LC-MS (Method 1): Rₜ=0.80 min; MS (ESIpos): m/z=299 [M+H]⁺

¹H-NMR (400 MHz, DMSO-d₆): δ [ppm]=1.70 (d, 3H), 5.83 (q, 1H), 6.01 (s, 2H), 7.39 (d, 1H), 7.63 (d, 1H).

### Intermediate 422

### 5-bromo-3-[(1R)-1-(2-methyl-1,3-thiazol-4-yl)ethoxy]pyridin-2-amine

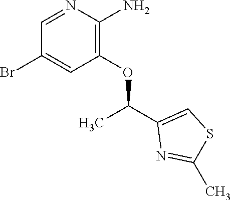 [SMILES: Cc1nc([C@@H](C)Oc2cc(Br)cnc2N)cs1]

5-Bromo-3-[(1R)-1-(2-methyl-1,3-thiazol-4-yl)ethoxy]-2-nitropyridine (183 mg, 531 μmol) was dissolved in ethanol (2.8 mL). Acetic acid (1.4 mL) and iron (119 mg, 2.12 mmol) were added and the mixture was stirred for 2 h at 80° C. The reaction mixture was allowed to cool down, diluted with ethyl acetate and filtered. The filtrate was concentrated and diluted in ethyl acetate again. The organic phase was washed with saturated sodium bicarbonate solution and brine, dried over a hydrophobic filter and concentrated to give 158 mg (95% purity, 90% yield) of the title compound.

LC-MS (Method 1): Rₜ=1.02 min; MS (ESIpos): m/z=314 [M+H]⁺

¹H-NMR (400 MHz, DMSO-d₆): δ [ppm]=1.59 (d, 3H), 2.64 (s, 3H), 5.58 (q, 1H), 5.95 (s, 2H), 7.25 (d, 1H), 7.52 (s, 1H), 7.56 (d, 1H).

### Intermediate 423

### (rac)-5-bromo-3-[1-(4-methyl-1,3-thiazol-2-yl)ethoxy]pyridin-2-amine

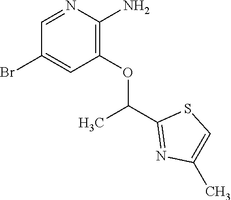 [SMILES: Cc1csc(C(C)Oc2cc(Br)cnc2N)n1]

(Rac)-5-Bromo-3-[1-(4-methyl-1,3-thiazol-2-yl)ethoxy]-2-nitropyridine (186 mg, 540 μmol) was dissolved in ethanol (2.9 mL). Acetic acid (1.4 mL) and iron (121 mg, 2.16 mmol) were added and the mixture was stirred for 1.5 h at 80° C. The reaction mixture was allowed to cool down, diluted with ethyl acetate and filtered. The filtrate was concentrated and diluted in ethyl acetate again. The organic phase was washed with saturated sodium bicarbonate solution and brine, dried over a hydrophobic filter and concentrated to give 129 mg (91% purity, 69% yield) of the title compound.

LC-MS (Method 1): Rₜ=1.04 min; MS (ESIpos): m/z=314 [M+H]⁺

¹H-NMR (400 MHz, DMSO-d₆): δ [ppm]=1.67 (d, 3H), 2.35 (d, 3H), 5.83 (q, 1H), 6.01 (s, 2H), 7.26 (d, 1H), 7.28 (d, 1H), 7.59 (d, 1H).

### Intermediate 424

### (rac)-bromo-3-[1-(1-methyl-1H-1,2,3-triazol-4-yl)ethoxy]pyridin-2-amine

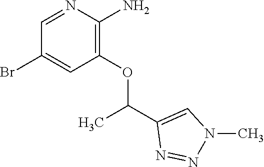 [SMILES: CC(Oc1cc(Br)cnc1N)c1cn(C)nn1]

(Rac)-5-Bromo-3-[1-(1-methyl-1H-1,2,3-triazol-4-yl)ethoxy]-2-nitropyridine (319 mg, 972 μmol) was dissolved in ethanol (5.2 mL). Acetic acid (2.6 mL) and iron (217 mg, 3.89 mmol) were added and the mixture was stirred for 3 h at 80° C. The reaction mixture was allowed to cool down, diluted with ethyl acetate and filtered. The filtrate was concentrated and diluted in ethyl acetate again. The organic phase was washed with saturated sodium bicarbonate solution and brine, dried over a hydrophobic filter and concentrated to give 281 mg (75% purity, 73% yield) of the title compound.

LC-MS (Method 1): Rₜ=0.77 min; MS (ESIpos): m/z=298 [M+H]⁺

¹H-NMR (400 MHz, DMSO-d₆): δ [ppm]=1.62 (d, 3H), 4.02 (s, 3H), 5.66 (q, 1H), 5.90 (s, 2H), 7.35 (d, 1H), 7.55 (d, 1H), 8.15 (s, 1H).

### Intermediate 425

### (rac)-5-bromo-3-{1-[3-(methanesulfonyl)phenyl]ethoxy}pyridin-2-amine

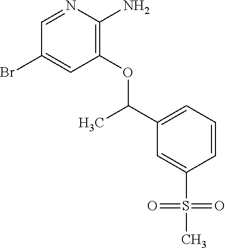 [SMILES: CC(Oc1cc(Br)cnc1N)c1cccc(S(C)(=O)=O)c1]

(Rac)-5-Bromo-3-{1-[3-(methanesulfonyl)phenyl]ethoxy}-2-nitropyridine (1.50 g, 3.74 mmol) was dissolved in methanol (19 mL) and acetic acid (19 mL). Iron (626 mg, 11.2 mmol) was added and the mixture was stirred for 1 h at 85° C. The reaction mixture was filtered and concentrated. The residue was purified by column chromatography (silica gel, hexane/ethyl acetate (0-90%) gradient) to give 989 mg (71% yield) of the title compound.

LC-MS (Method 1): Rₜ=0.97 min; MS (ESIpos): m/z=371 [M+H]⁺

¹H-NMR (400 MHz, DMSO-d6) δ[ppm]: 1.345 (1.59), 1.352 (0.48), 1.361 (1.64), 1.572 (2.31), 1.588 (2.28), 1.907 (16.00), 3.144 (0.98), 3.166 (9.45), 3.191 (1.04), 3.204 (4.02), 3.215 (7.57), 3.228 (0.62), 3.338 (0.63), 5.739 (0.54), 5.755 (0.58), 6.117 (1.35), 7.196 (1.07), 7.202 (1.09), 7.516 (1.83), 7.521 (1.80), 7.595 (0.52), 7.627 (0.50), 7.647 (0.97), 7.666 (0.77), 7.835 (1.08), 7.839 (1.32), 7.854 (1.05), 7.859 (1.18), 7.906 (0.45), 8.061 (0.64), 8.065 (1.07), 8.070 (0.58).

### Intermediate 426

### (rac)-5-bromo-3-[1-(1,3-oxazol-4-yl)ethoxy]pyridin-2-amine

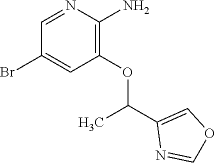 [SMILES: CC(Oc1cc(Br)cnc1N)c1cocn1]

(Rac)-5-Bromo-2-nitro-3-[1-(1,3-oxazol-4-yl)ethoxy]pyridine (185 mg, 589 μmol) was dissolved in ethanol (3.1 mL). Acetic acid (1.6 mL) and iron (98.7 mg, 1.77 mmol) were added and the mixture was stirred for 1.5 h at 80° C. The reaction mixture was allowed to cool down, diluted with ethyl acetate and filtered. The filtrate was concentrated and diluted in ethyl acetate again. The organic phase was washed with saturated sodium bicarbonate solution and brine, dried over a hydrophobic filter and concentrated to give 140 mg (100% purity, 84% yield) of the title compound.

LC-MS (Method 2): Rₜ=0.71 min; MS (ESIpos): m/z=284 [M+H]⁺

¹H-NMR (400 MHz, DMSO-d₆): δ [ppm]=1.57 (br d, 3H), 5.46-5.54 (m, 1H), 5.91 (br s, 2H), 7.34 (br s, 1H), 7.58 (br s, 1H), 8.21 (br s, 1H), 8.38 (br s, 1H).

### Intermediate 427

### (rac)-5-bromo-3-[1-(1-propyl-1H-pyrazol-5-yl)ethoxy]pyridin-2-amine (Enantiomer 1 and Enantiomer 2)

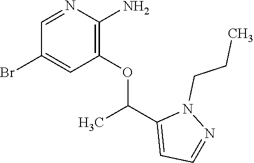 [SMILES: CCCn1nccc1C(C)Oc1cc(Br)cnc1N]

(Rac)-5-Bromo-2-nitro-3-[1-(1-propyl-1H-pyrazol-5-yl)ethoxy]pyridine (675 mg, 1.90 mmol) was dissolved in methanol (9 mL), THF (9 mL) and water (18 mL). Iron (1.06 g, 19.0 mmol) and ammonium chloride (1.01 g, 19 mmol) were added and the mixture was stirred for 3 h at 80° C. The reaction mixture was allowed to cool down, filtered and the filtrate was extracted with ethyl acetate. The combined organic layers were washed with brine, dried over sodium sulfate, filtered and concentrated. The crude product was purified by column chromatography (silica gel, hexane/ethyl acetate (0-50%) gradient) followed by a second column chromatography (silica gel NH2, dichloromethane (100%) isocratic) to give 159 mg (26% yield) of the title compound.

LC-MS (Method 1): Rₜ=1.05 min; MS (ESIpos): m/z=325 [M+H]⁺

¹H-NMR (400 MHz, DMSO-d6) δ[ppm]: 0.781 (5.44), 0.799 (13.02), 0.818 (6.09), 0.825 (7.33), 0.844 (16.00), 0.862 (7.49), 1.172 (0.46), 1.399 (14.58), 1.415 (15.23), 1.579 (9.34), 1.595 (9.43), 1.670 (0.41), 1.688 (1.53), 1.707 (2.68), 1.715 (0.95), 1.725 (2.71), 1.734 (3.07), 1.743 (1.63), 1.752 (5.58), 1.762 (0.57), 1.771 (5.24), 1.789 (2.54), 1.808 (0.51), 1.988 (0.78), 2.518 (2.89), 2.523 (1.96), 3.982 (0.54), 3.999 (1.09), 4.016 (2.28), 4.021 (3.13), 4.034 (4.60), 4.038 (5.18), 4.052 (2.35), 4.057 (5.88), 4.075 (1.88), 4.091 (1.06), 4.109 (0.50), 4.769 (0.41), 4.785 (1.54), 4.800 (2.22), 4.816 (1.59), 4.832 (0.42), 5.234 (6.68), 5.248 (6.16), 5.759 (9.80), 5.777 (0.56), 5.793 (1.97), 5.809 (1.95), 5.825 (0.57), 5.961 (4.35), 6.110 (4.33), 6.114 (4.37), 6.383 (4.69), 6.388 (4.79), 7.294 (4.69), 7.299 (5.91), 7.305 (3.88), 7.383 (4.81), 7.388 (4.72), 7.564 (5.55), 7.569 (5.20).

### Intermediate 428

### (rac)-5-bromo-3-[1-(pyrimidin-4-yl)ethoxy]pyridin-2-amine

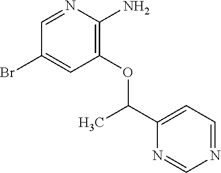 [SMILES: CC(Oc1cc(Br)cnc1N)c1ccncn1]

(Rac)-4-{1-[(5-Bromo-2-nitropyridin-3-yl)oxy]ethyl}pyrimidine (1.13 g, 3.47 mmol) was dissolved in ethanol (30 mL). Acetic acid (9.2 mL) and iron (1.16 g, 20.8 mmol]) were added and stirred for 4 h at 80° C. The reaction mixture was allowed to cool down, diluted with ethyl acetate and filtered. The filtrate was concentrated and diluted in ethyl acetate again. The organic phase was washed with saturated sodium bicarbonate solution and water, dried and concentrated. The residue was purified by column chromatography (silica gel, dichloromethane/ethanol (0-10%) gradient) followed by column chromatography (silica gel, ethyl acetate/ethanol (0-10%) gradient) to give 757 mg (74% yield) of the title compound.

LC-MS (Method 1): Rₜ=0.84 min; MS (ESIpos): m/z=295 [M+H]⁺

¹H-NMR (400 MHz, DMSO-d6) δ[ppm]: 1.154 (3.26), 1.172 (6.62), 1.189 (3.20), 1.581 (15.99), 1.597 (16.00), 1.987 (12.12), 2.470 (0.56), 2.518 (2.29), 2.523 (1.48), 3.999 (0.87), 4.017 (2.67), 4.035 (2.68), 4.053 (0.87), 5.540 (0.96), 5.556 (3.27), 5.572 (3.26), 5.589 (0.93), 6.147 (6.32), 7.138 (5.61), 7.143 (5.68), 7.570 (8.18), 7.575 (7.98), 7.679 (3.51), 7.683 (3.57), 7.692 (3.67), 7.695 (3.61), 8.819 (7.19), 8.832 (6.97), 9.182 (6.99), 9.186 (6.86).

### Intermediate 429

### 5-bromo-3-{[1-(2,4-dimethylpyrimidin-5-yl)ethyl]oxy}pyridin-2-amine (Enantiomer 2)

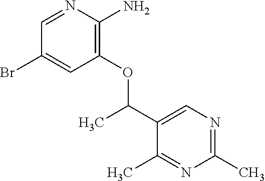 [SMILES: Cc1ncc(C(C)Oc2cc(Br)cnc2N)c(C)n1]

5-{1-[(5-Bromo-2-nitropyridin-3-yl)oxy]ethyl}-2,4-dimethylpyrimidine (enantiomer 2) (406 mg, 1.15 mmol) was dissolved in ethanol (10 mL). Acetic acid (3.1 mL) and iron (385 mg, 6.90 mmol) were added and the mixture was stirred for 3 h at 80° C. The reaction mixture was allowed to cool down, diluted with ethyl acetate and filtered. The filtrate was concentrated and diluted in ethyl acetate again. The organic phase was washed with saturated sodium bicarbonate solution and water, dried and concentrated. The residue was purified by column chromatography (silica gel, dichloromethane/ethanol (0-10%) gradient) to give 363 mg (98% yield) of the title compound.

[α]²⁰_{D}: −3.3° (c=1.00, methanol)

LC-MS (Method 1): Rₜ=0.89 min; MS (ESIpos): m/z=323 [M+H]⁺

¹H NMR (400 MHz, DMSO-d₆) δ ppm=1.56 (d, 3H), 2.53 (s, 3H), 5.78 (q, 1H), 6.13 (s, 2H), 7.22 (d, 1H), 7.55 (d, 1H), 8.73 (s, 1H).

### Intermediate 430

### 5-bromo-3-{[1-(2,4-dimethylpyrimidin-5-yl)ethyl]oxy}pyridin-2-amine (Enantiomer 1)

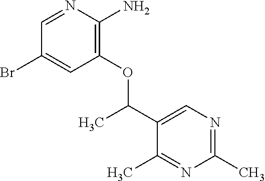 [SMILES: Cc1ncc(C(C)Oc2cc(Br)cnc2N)c(C)n1]

5-{1-[(5-Bromo-2-nitropyridin-3-yl)oxy]ethyl}-2,4-dimethylpyrimidine (enantiomer 1) (414 mg, 1.17 mmol) was dissolved in ethanol (10 mL). Acetic acid (3.1 mL) and iron (393 mg, 7.03 mmol) were added and the mixture was stirred for 3 h at 80° C. The reaction mixture was allowed to cool down, diluted with ethyl acetate and filtered. The filtrate was concentrated and diluted in ethyl acetate again. The organic phase was washed with saturated sodium bicarbonate solution and water, dried and concentrated. The residue was purified by column chromatography (silica gel, dichloromethane/ethanol (0-10%) gradient) to give 383 mg (101% yield) of the title compound.

[α]²⁰_{D}: +4.1° (c=1.00, methanol)

LC-MS (Method 1): Rₜ=0.89 min; MS (ESIpos): m/z=323 [M+H]⁺

¹H NMR (400 MHz, DMSO-d₆) δ ppm=1.56 (d, 3H), 2.53 (s, 3H), 5.78 (q, 1H), 6.13 (s, 2H), 7.22 (d, 1H), 7.55 (d, 1H), 8.73 (s, 1H).

### Intermediate 431

### (rac)-5-bromo-3-{1-[4-(methanesulfonyl)phenyl]ethoxy}pyridin-2-amine

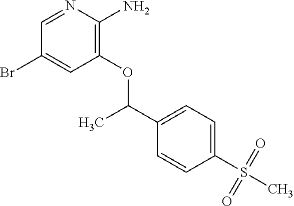 [SMILES: CC(Oc1cc(Br)cnc1N)c1ccc(S(C)(=O)=O)cc1]

(Rac)-5-Bromo-3-{1-[4-(methanesulfonyl)phenyl]ethoxy}-2-nitropyridine (450 mg, 1.12 mmol) was dissolved in methanol (5.5 mL) and acetic acid (5.5 mL). Iron (188 mg, 3.36 mmol) was added and the mixture was stirred for 1 h at 85° C. The reaction mixture was allowed to cool down, filtered and concentrated. The residue was purified by column chromatography (silica gel, hexane/ethyl acetate (0-100%) gradient) to give 298 mg (72% yield) of the title compound.

LC-MS (Method 1): Rₜ=0.98 min; MS (ESIpos): m/z=371 [M+H]⁺

¹H-NMR (400 MHz, DMSO-d6) δ[ppm]: 1.154 (1.42), 1.172 (3.08), 1.190 (1.51), 1.326 (0.51), 1.343 (0.53), 1.553 (4.75), 1.569 (4.77), 1.987 (4.26), 2.518 (2.30), 2.523 (1.55), 3.188 (1.34), 3.214 (16.00), 4.017 (0.96), 4.035 (0.98), 5.736 (1.03), 5.751 (1.04), 6.125 (2.81), 7.137 (2.23), 7.142 (2.28), 7.516 (3.93), 7.521 (3.87), 7.735 (2.88), 7.740 (0.97), 7.752 (1.07), 7.756 (3.74), 7.902 (0.59), 7.906 (4.29), 7.912 (1.20), 7.924 (1.04), 7.928 (3.40), 7.932 (0.44). Borylation

### Intermediate 432

### {6-amino-5-[1-(6-methylpyridin-2-yl)ethoxy]pyridin-3-yl}boronic acid (Enantiomer 1)

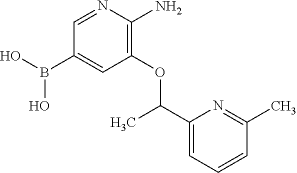 [SMILES: Cc1cccc(C(C)Oc2cc(B(O)O)cnc2N)n1]

5-Bromo-3-[1-(6-methylpyridin-2-yl)ethoxy]pyridin-2-amine (enantiomer 1) (Intermediate 376 and Intermediate 377, 96.0 mg, 312 μmol), 4,4,4′,4′,5,5,5′,5′-octamethyl-2,2′-bi-1,3,2-dioxaborolane (94.9 mg, 374 μmol) and potassium acetate (91.7 mg, 935 μmol) were suspended in 1,4-dioxane (1.9 mL) and the reaction mixture was degassed. Pd(dppf)Cl₂ (11.4 mg, 15.6 μmol) was added and the mixture was stirred under argon for 2 h at 100° C. The reaction mixture was cooled to rt, diluted with ethyl acetate and filtered. The filtrate was washed with water and brine, dried over sodium sulfate, filtered and concentrated under reduced pressure to give 182 mg (22% purity, 47% yield) of crude material that was used without further purification in the next step.

LC-MS (Method 1): Rₜ=0.56 min; MS (ESIpos): m/z=274 [M+H]⁺

### Intermediate 433

### {6-amino-5-[1-(6-methylpyridin-2-yl)ethoxy]pyridin-3-yl}boronic acid (Enantiomer 2)

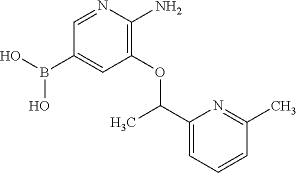 [SMILES: Cc1cccc(C(C)Oc2cc(B(O)O)cnc2N)n1]

The compound was prepared similarly to Intermediate 432 starting from 5-bromo-3-[1-(6-methylpyridin-2-yl)ethoxy]pyridin-2-amine (enantiomer 2) (Intermediate 376 and Intermediate 377, 104 mg, 337 μmol) to give 158 mg (35% purity, 60% yield) of the title compound.

LC-MS (Method 1): Rₜ=0.56 min; MS (ESIpos): m/z=274 [M+H]⁺

### Intermediate 434

### {6-amino-5-[1-(5-methylpyridin-2-yl)ethoxy]pyridin-3-yl}boronic acid (Enantiomer 1)

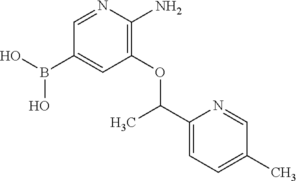 [SMILES: Cc1ccc(C(C)Oc2cc(B(O)O)cnc2N)nc1]

5-Bromo-3-[1-(5-methylpyridin-2-yl)ethoxy]pyridin-2-amine (enantiomer 1) (Intermediate 378 and Intermediate 379, 76.0 mg, 247 μmol), 4,4,4′,4′,5,5,5′,5′-octamethyl-2,2′-bi-1,3,2-dioxaborolane (75 mg, 295 μmol) and potassium acetate (72.6 mg, 739 μmol) were suspended in 1,4-dioxane (1.3 mL) and the reaction mixture was degassed. Pd(dppf)Cl₂ (9 mg, 12 μmol) was added and the mixture was stirred under argon for 2 h at 100° C. 4,4,4′,4′,5,5,5′,5′-Octamethyl-2,2′-bi-1,3,2-dioxaborolane (25 mg, 73.7 μmol) and Pd(dppf)Cl₂ (5 mg, 6.6 μmol) were added again and the mixture was stirred for 3 h at 100° C. The reaction mixture was allowed to cool down, diluted with ethyl acetate and filtered. The filtrate was washed with water, brine, dried over sodium sulfate, filtered and concentrated under reduced pressure to give 161 mg (25% purity, 60% yield) of the title compound which was used without further purification in the next step.

LC-MS (Method 1): Rₜ=0.60 min; MS (ESIpos): m/z=274 [M+H]⁺

### Intermediate 435

### {6-amino-5-[1-(5-methylpyridin-2-yl)ethoxy]pyridin-3-yl}boronic acid (Enantiomer 2)

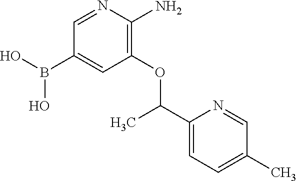 [SMILES: Cc1ccc(C(C)Oc2cc(B(O)O)cnc2N)nc1]

The compound was prepared similarly to Intermediate 434 starting from 5-bromo-3-[1-(5-methylpyridin-2-yl)ethoxy]pyridin-2-amine (enantiomer 2) (Intermediate 378 and Intermediate 379, 80.0 mg, 260 μmol) to give 178.6 mg (25% purity, 63% yield) of the title compound.

LC-MS (Method 1): Rₜ=0.60 min; MS (ESIpos): m/z=274 [M+H]⁺

### Intermediate 436

### {6-amino-5-[1-(4-methylpyridin-2-yl)ethoxy]pyridin-3-yl}boronic acid (Enantiomer 1)

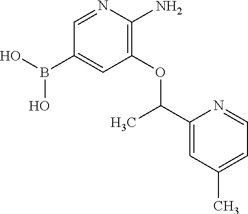 [SMILES: Cc1ccnc(C(C)Oc2cc(B(O)O)cnc2N)c1]

5-Bromo-3-[1-(4-methylpyridin-2-yl)ethoxy]pyridin-2-amine (enantiomer 1) (Intermediate 380 and Intermediate 381, 55.0 mg, 178 μmol), 4,4,4′,4′,5,5,5′,5′-octamethyl-2,2′-bi-1,3,2-dioxaborolane (54 mg, 214 μmol) and potassium acetate (52.5 mg, 535 μmol) were suspended in 1,4-dioxane (1 mL) and the reaction mixture was degassed. Pd(dppf)Cl₂ (6.5 mg, 8.9 μmol) was added and the mixture was stirred under argon for 2 h at 100° C. 4,4,4′,4′,5,5,5′,5′-Octamethyl-2,2′-bi-1,3,2-dioxaborolane (54 mg, 214 μmol) and Pd(dppf)Cl₂ (6.5 mg, 8.9 μmol) were added again and the mixture was stirred for 3 h at 100° C. The reaction mixture was allowed to cool down, diluted with ethyl acetate and filtered. The filtrate was washed with water and brine, dried over sodium sulfate, filtered and concentrated under reduced pressure to give 142.7 mg (20% purity, 58% yield) of the title compound.

LC-MS (Method 1): Rₜ=0.58 min; MS (ESIpos): m/z=274 [M+H]⁺

### Intermediate 437

### {6-amino-5-[1-(4-methylpyridin-2-yl)ethoxy]pyridin-3-yl}boronic acid (Enantiomer 2)

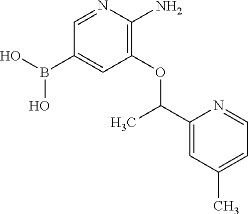 [SMILES: Cc1ccnc(C(C)Oc2cc(B(O)O)cnc2N)c1]

The compound was prepared similarly to Intermediate 436 starting from 5-bromo-3-[1-(4-methylpyridin-2-yl)ethoxy]pyridin-2-amine (enantiomer 2) (Intermediate 380 and Intermediate 381, 54.0 mg, 175 μmol) to give 143.5 mg (20% purity, 60% yield) of the title compound.

LC-MS (Method 1): Rₜ=0.58 min; MS (ESIpos): m/z=274 [M+H]⁺

### Intermediate 438

### {6-amino-5-[1-(3-methylpyridin-2-yl)ethoxy]pyridin-3-yl}boronic acid (Enantiomer 1)

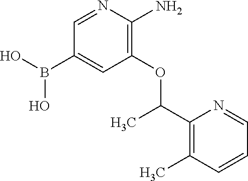 [SMILES: Cc1cccnc1C(C)Oc1cc(B(O)O)cnc1N]

5-Bromo-3-[1-(3-methylpyridin-2-yl)ethoxy]pyridin-2-amine (enantiomer 1) (Intermediate 382 and Intermediate 383, 34.0 mg, 110 μmol), 4,4,4′,4′,5,5,5′,5′-octamethyl-2,2′-bi-1,3,2-dioxaborolane (34 mg, 132 μmol) and potassium acetate (32.4 mg, 331 μmol) were suspended in 1,4-dioxane (0.5 mL) and the reaction mixture was degassed. Pd(dppf)Cl₂ (4 mg, 5.5 μmol) was added and the mixture was stirred under argon for 2 h at 100° C. 4,4,4′,4′,5,5,5′,5′-Octamethyl-2,2′-bi-1,3,2-dioxaborolane (34 mg, 110 μmol) and Pd(dppf)Cl₂ (4 mg, 5.5 μmol) were added again and the mixture was stirred for 3 h at 100° C. The reaction mixture was allowed to cool down, diluted with ethyl acetate and filtered. The filtrate was washed with water, brine, dried over sodium sulfate, filtered and concentrated under reduced pressure to give 87.6 mg (15% purity, 43% yield) of the title compound.

LC-MS (Method 1): Rₜ=0.56 min; MS (ESIpos): m/z=274 [M+H]⁺

### Intermediate 439

### {6-amino-5-[1-(3-methylpyridin-2-yl)ethoxy]pyridin-3-yl}boronic acid (Enantiomer 2)

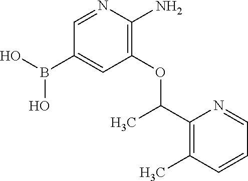 [SMILES: Cc1cccnc1C(C)Oc1cc(B(O)O)cnc1N]

The compound was prepared similarly to Intermediate 438 starting from 5-bromo-3-[1-(3-methylpyridin-2-yl)ethoxy]pyridin-2-amine (enantiomer 2) (Intermediate 382 and Intermediate 383, 34.0 mg, 110 μmol) to give 83 mg (20% purity, 55% yield) of the title compound.

LC-MS (Method 1): Rₜ=0.55 min; MS (ESIpos): m/z=274 [M+H]⁺

### Intermediate 440

### 3-[1-(1,4-dimethyl-1H-pyrazol-5-yl)ethoxy]-5-(4,4,5,5-tetramethyl-1,3,2-dioxaborolan-2-yl)pyridin-2-amine (Enantiomer 1)

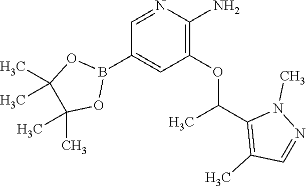 [SMILES: Cc1cnn(C)c1C(C)Oc1cc(B2OC(C)(C)C(C)(C)O2)cnc1N]

5-Bromo-3-[1-(1,4-dimethyl-1H-pyrazol-5-yl)ethoxy]pyridin-2-amine (enantiomer 1) (Intermediate 384 and Intermediate 385, 88.0 mg, 283 μmol), 4,4,4′,4′,5,5,5′,5′-octamethyl-2,2′-bi-1,3,2-dioxaborolane (86 mg, 339 μmol) and potassium acetate (83 mg, 848 μmol) were suspended in 1,4-dioxane (1.9 mL) and the reaction mixture was degassed. Pd(dppf)Cl₂ (10 mg, 14 μmol) was added and the mixture was stirred under argon for 2 h at 100° C. The reaction mixture was allowed to cool down, diluted with ethyl acetate and filtered. The filtrate was washed with water, brine, dried over sodium sulfate, filtered and concentrated under reduced pressure to give 135 mg (50% purity, 66% yield) of the title compound.

LC-MS (Method 1): Rₜ=0.92 min; MS (ESIpos): m/z=359 [M+H]⁺

### Intermediate 441

### {6-amino-5-[1-(1,4-dimethyl-1H-pyrazol-5-yl)ethoxy]pyridin-3-yl}boronic acid (Enantiomer 1)

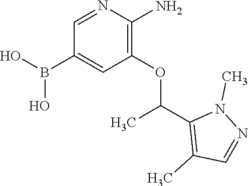 [SMILES: Cc1cnn(C)c1C(C)Oc1cc(B(O)O)cnc1N]

5-Bromo-3-[1-(1,4-dimethyl-1H-pyrazol-5-yl)ethoxy]pyridin-2-amine (enantiomer 2) (Intermediate 384 and Intermediate 385, 72.0 mg, 231 μmol), 4,4,4′,4′,5,5,5′,5′-octamethyl-2,2′-bi-1,3,2-dioxaborolane (70.5 mg, 278 μmol) and potassium acetate (68.1 mg, 694 μmol) were suspended in 1,4-dioxane (1.6 mL) and the reaction mixture was degassed. Pd(dppf)Cl₂ (8.46 mg, 11.6 μmol) was added and the mixture was stirred under argon for 2 h at 100° C. 4,4,4′,4′,5,5,5′,5′-octamethyl-2,2′-bi-1,3,2-dioxaborolane (35 mg, 139 μmol) and Pd(dppf)Cl₂ (5 mg, 6.8 μmol) were added again and the mixture was stirred for 1 h at 100° C. The reaction mixture was allowed to cool down, diluted with ethyl acetate and filtered. The filtrate was washed with water, brine, dried over sodium sulfate, filtered and concentrated under reduced pressure to give 167 mg (20% purity, 52% yield) of the title compound.

LC-MS (Method 1): Rₜ=0.53 min; MS (ESIpos): m/z=277 [M+H]⁺

### Intermediate 442

### 3-[1-(1,4-dimethyl-1H-pyrazol-5-yl)ethoxy]-5-(4,4,5,5-tetramethyl-1,3,2-dioxaborolan-2-yl)pyridin-2-amine (Enantiomer 2)

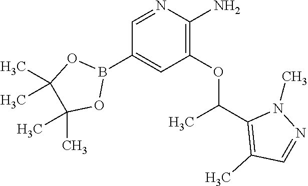 [SMILES: Cc1cnn(C)c1C(C)Oc1cc(B2OC(C)(C)C(C)(C)O2)cnc1N]

The compound was prepared similarly to Intermediate 440 starting from 5-bromo-3-[1-(1,4-dimethyl-1H-pyrazol-5-yl)ethoxy]pyridin-2-amine (enantiomer 2) (Intermediate 384 and Intermediate 385, 69.0 mg, 222 μmol) to give 110 mg (50% purity, 69% yield) of the title compound.

LC-MS (Method 1): Rₜ=0.92 min; MS (ESIpos): m/z=359 [M+H]⁺

### Intermediate 443

### {6-amino-5-[1-(1,4-dimethyl-1H-pyrazol-5-yl)ethoxy]pyridin-3-yl}boronic acid (Enantiomer 2)

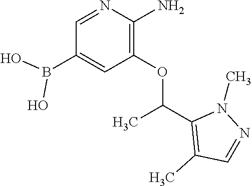 [SMILES: Cc1cnn(C)c1C(C)Oc1cc(B(O)O)cnc1N]

The compound was prepared similarly to Intermediate 440 starting from 5-bromo-3-[1-(1,4-dimethyl-1H-pyrazol-5-yl)ethoxy]pyridin-2-amine (enantiomer 2) (Intermediate 384 and Intermediate 385, 69.0 mg, 222 μmol) to give 115 mg (25% purity, 50% yield) of the title compound.

LC-MS (Method 1): Rₜ=0.54 min; MS (ESIpos): m/z=277 [M+H]⁺

### Intermediate 444

### {6-amino-5-[(1S)-1-(2,6-difluorophenyl)ethoxy]pyridin-3-yl}boronic acid

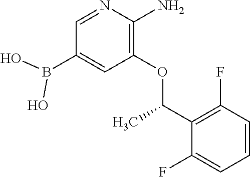 [SMILES: C[C@H](Oc1cc(B(O)O)cnc1N)c1c(F)cccc1F]

5-Bromo-3-[(1S)-1-(2,6-difluorophenyl)ethoxy]pyridin-2-amine (230 mg, 699 μmol), 4,4,4′,4′,5,5,5′,5′-octamethyl-2,2′-bi-1,3,2-dioxaborolane (213 mg, 839 μmol) and potassium acetate (206 mg, 2.10 mmol) were suspended in 1,4-dioxane (4.9 mL) and the reaction mixture was degassed. Pd(dppf)Cl₂ (25.6 mg, 34.9 μmol) was added and the mixture was stirred under argon for 2 h at 100° C. The reaction mixture was allowed to cool down, diluted with ethyl acetate and filtered. The filtrate was washed with water and brine, dried over sodium sulfate, filtered and concentrated under reduced pressure to give 464 mg (30% purity, 67% yield) of the title compound.

LC-MS (Method 1): Rₜ=0.78 min; MS (ESIpos): m/z=295 [M+H]⁺

### Intermediate 445

### {6-amino-5-[(1R)-1-(5-fluoropyridin-2-yl)ethoxy]pyridin-3-yl}boronic acid

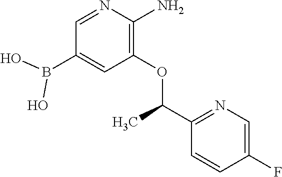 [SMILES: C[C@@H](Oc1cc(B(O)O)cnc1N)c1ccc(F)cn1]

5-Bromo-3-[(1R)-1-(5-fluoropyridin-2-yl)ethoxy]pyridin-2-amine (198 mg, 634 μmol), 4,4,4′,4′,5,5,5′,5′-octamethyl-2,2′-bi-1,3,2-dioxaborolane (193 mg, 761 μmol) and potassium acetate (187 mg, 1.90 mmol) were suspended in 1,4-dioxane (4.4 mL) and the reaction mixture was degassed. Pd(dppf)Cl₂ (23.2 mg, 31.7 μmol) was added and the mixture was stirred under argon for 2 h at 100° C. The reaction mixture was allowed to cool down, diluted with ethyl acetate and filtered. The filtrate was washed with water and brine, dried over sodium sulfate, filtered and concentrated under reduced pressure to give 423 mg (33% purity, 79% yield) of the title compound.

LC-MS (Method 1): Rₜ=0.55 min; MS (ESIpos): m/z=278 [M+H]⁺

### Intermediate 446

### {6-amino-5-[(1R)-1-(3-fluoropyridin-2-yl)ethoxy]pyridin-3-yl}boronic acid

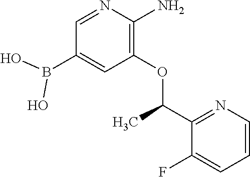 [SMILES: C[C@@H](Oc1cc(B(O)O)cnc1N)c1ncccc1F]

The compound was prepared similarly to Intermediate 445 starting from 5-bromo-3-[(1R)-1-(3-fluoropyridin-2-yl)ethoxy]pyridin-2-amine (193 mg, 618 μmol) to give 383 mg (35% purity, 78% yield) of the title compound.

LC-MS (Method 1): Rₜ=0.53 min; MS (ESIpos): m/z=278 [M+H]⁺

### Intermediate 447

### {6-amino-5-[(1R)-1-(2,6-difluorophenyl)ethoxy]pyridin-3-yl}boronic acid

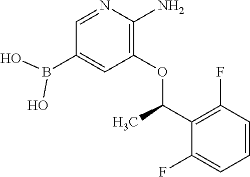 [SMILES: C[C@@H](Oc1cc(B(O)O)cnc1N)c1c(F)cccc1F]

The compound was prepared similarly to Intermediate 445 starting from 5-bromo-3-[(1R)-1-(2,6-difluorophenyl)ethoxy]pyridin-2-amine (204 mg, 620 μmol) to give 154 mg (32% purity, 68% yield) of the title compound.

LC-MS (Method 1): Rₜ=0.78 min; MS (ESIpos): m/z=295 [M+H]⁺

### Intermediate 448

### (rac)-2-(1-{[2-amino-5-(4,4,5,5-tetramethyl-1,3,2-dioxaborolan-2-yl)pyridin-3-yl]oxy}ethyl)benzonitrile

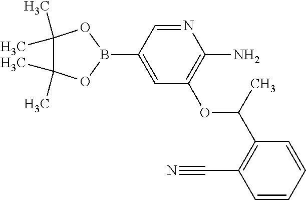 [SMILES: CC(Oc1cc(B2OC(C)(C)C(C)(C)O2)cnc1N)c1ccccc1C#N]

(Rac)-2-{1-[(2-Amino-5-bromopyridin-3-yl)oxy]ethyl}benzonitrile (142 mg, 446 μmol), 4,4,4′,4′,5,5,5′,5′-octamethyl-2,2′-bi-1,3,2-dioxaborolane (125 mg, 491 μmol) and potassium acetate (131 mg, 1.34 mmol) were suspended in 1,4-dioxane (7 mL) and the reaction mixture was degassed. Pd(dppf)Cl₂ (32.7 mg, 44.6 μmol) was added and the mixture was stirred under argon for 2 h at 100° C. The cold reaction mixture was allowed to cool down, diluted with ethyl acetate and water. The aqueous phase was extracted 3 times with ethyl acetate. The combined organic layers were dried over a hydrophobic filter and concentrated to give 321 mg (40% purity, 79% yield) of the title compound.

LC-MS (Method 1): Rₜ=0.65 min; MS (ESIpos): m/z=284 [M+H]⁺

¹H-NMR (400 MHz, DMSO-d6) δ[ppm]: 1.066 (3.03), 1.156 (16.00), 1.164 (8.36), 1.221 (1.61), 1.229 (1.66), 1.292 (0.42), 1.610 (0.49), 1.626 (0.50), 1.642 (0.34), 1.657 (0.32), 1.906 (0.59), 2.518 (0.27), 2.523 (0.16), 3.565 (3.34), 3.940 (0.32), 6.279 (0.19), 7.015 (0.22), 7.017 (0.22), 7.502 (0.21), 7.505 (0.20), 7.521 (0.18), 7.524 (0.17), 7.559 (0.18), 7.564 (0.17), 7.728 (0.20), 7.788 (0.39), 7.791 (0.29), 7.943 (0.63).

### Intermediate 449

### {6-amino-5-[(1R)-1-(3-cyanophenyl)ethoxy]pyridin-3-yl}boronic acid

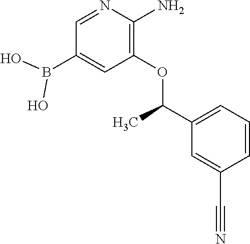 [SMILES: C[C@@H](Oc1cc(B(O)O)cnc1N)c1cccc(C#N)c1]

3-{(1R)-1-[(2-Amino-5-bromopyridin-3-yl)oxy]ethyl}benzonitrile (133 mg, 418 μmol), 4,4,4′,4′,5,5,5′,5′-octamethyl-2,2′-bi-1,3,2-dioxaborolane (127 mg, 502 μmol) and potassium acetate (123 mg, 1.25 mmol) were suspended in 1,4-dioxane (2.9 mL) and the reaction mixture was degassed. Pd(dppf)Cl₂ (15.3 mg, 20.9 μmol) was added and the mixture was stirred under argon for 2 h at 100° C. The reaction mixture was allowed to cool down, diluted with ethyl acetate and filtered. The filtrate was washed with water and brine, dried over sodium sulfate, filtered and concentrated under reduced pressure to give 223 mg (38% purity, 72% yield) of the title compound.

LC-MS (Method 1): Rₜ=0.68 min; MS (ESIpos): m/z=284 [M+H]⁺

### Intermediate 450

### {6-amino-5-[(1R)-1-(4-cyanophenyl)ethoxy]pyridin-3-yl}boronic acid

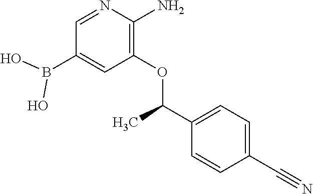 [SMILES: C[C@@H](Oc1cc(B(O)O)cnc1N)c1ccc(C#N)cc1]

The compound was prepared similarly to Intermediate 449 starting from 4-{(1R)-1-[(2-amino-5-bromopyridin-3-yl)oxy]ethyl}benzonitrile (117 mg, 368 μmol) to give 176 mg (45% purity, 76% yield) of the title compound.

LC-MS (Method 1): Rₜ=0.64 min; MS (ESIpos): m/z=284 [M+H]⁺

### Intermediate 451

### {6-amino-5-[(1R)-1-(3,5-difluoropyridin-4-yl)ethoxy]pyridin-3-yl}boronic acid

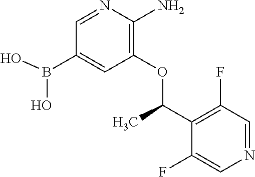 [SMILES: C[C@@H](Oc1cc(B(O)O)cnc1N)c1c(F)cncc1F]

The compound was prepared similarly to Intermediate 449 starting from 5-bromo-3-[(1R)-1-(3,5-difluoropyridin-4-yl)ethoxy]pyridin-2-amine (163 mg, 494 μmol) to give 256 mg (43% purity, 75% yield) of the title compound.

LC-MS (Method 1): Rₜ=0.55 min; MS (ESIpos): m/z=296 [M+H]⁺

### Intermediate 452

### (rac)-[6-amino-5-(1-phenylpropoxy)pyridin-3-yl]boronic acid

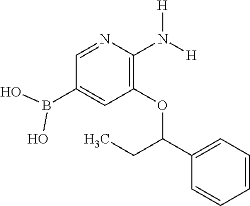 [SMILES: CCC(Oc1cc(B(O)O)cnc1N)c1ccccc1]

(Rac)-5-Bromo-3-(1-phenylpropoxy)pyridin-2-amine (320 mg, 1.04 mmol), 4,4,4′,4′,5,5,5′,5′-octamethyl-2,2′-bi-1,3,2-dioxaborolane (317 mg, 1.25 mmol) and potassium acetate (307 mg, 3.12 mmol) were suspended in 1,4-dioxane (9.7 mL) and the reaction mixture was degassed. Pd(dppf)Cl₂ (76.2 mg, 104 μmol) was added and the mixture was stirred under argon for 2 h at 100° C. The reaction mixture was allowed to cool down, diluted with ethyl acetate and water. The aqueous phase was extracted 3 times with ethyl acetate. The combined organic layers were dried over a hydrophobic filter and concentrated to give 551.1 mg (55% purity, 106% yield) of the title compound.

LC-MS (Method 1): Rₜ=0.84 min; MS (ESIpos): m/z=273 [M+H]⁺

### Intermediate 453

### {6-amino-5-[(1S)-1-(3-cyanophenyl)ethoxy]pyridin-3-yl}boronic acid

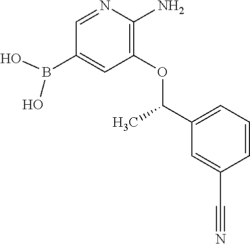 [SMILES: C[C@H](Oc1cc(B(O)O)cnc1N)c1cccc(C#N)c1]

3-{(1S)-1-[(2-Amino-5-bromopyridin-3-yl)oxy]ethyl}benzonitrile (164 mg, 515 μmol), 4,4,4′,4′,5,5,5′,5′-octamethyl-2,2′-bi-1,3,2-dioxaborolane (157 mg, 619 μmol) and potassium acetate (152 mg, 1.55 mmol) were suspended in 1,4-dioxane (3.6 mL) and the reaction mixture was degassed. Pd(dppf)Cl₂ (18.9 mg, 25.8 μmol) was added and the mixture was stirred under argon for 2 h at 100° C. The reaction mixture was allowed to cool down, diluted with ethyl acetate and filtered. The filtrate was washed with water and brine. The organic phase was dried over sodium sulfate, filtered and concentrated to give 260 mg (41% purity, 73% yield) of the title compound.

LC-MS (Method 1): Rₜ=0.65 min; MS (ESIpos): m/z=284 [M+H]⁺

### Intermediate 454

### {6-amino-5-[(1S)-1-(4-cyanophenyl)ethoxy]pyridin-3-yl}boronic acid

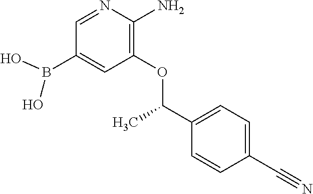 [SMILES: C[C@H](Oc1cc(B(O)O)cnc1N)c1ccc(C#N)cc1]

The compound was prepared similarly to Intermediate 453 starting from 4-{(1S)-1-[(2-amino-5-bromopyridin-3-yl)oxy]ethyl}benzonitrile (128 mg, 402 μmol) to give 210 mg (40% purity, 73% yield) of the title compound.

LC-MS (Method 1): Rₜ=0.64 min; MS (ESIpos): m/z=284 [M+H]⁺

### Intermediate 455

### {6-amino-5-[(1S)-1-(3,5-difluoropyridin-4-yl)ethoxy]pyridin-3-yl}boronic acid

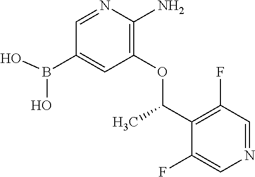 [SMILES: C[C@H](Oc1cc(B(O)O)cnc1N)c1c(F)cncc1F]

The compound was prepared similarly to Intermediate 453 starting from 5-bromo-3-[(1S)-1-(3,5-difluoropyridin-4-yl)ethoxy]pyridin-2-amine (150 mg, 454 μmol) to give 241 mg (41% purity, 74% yield) of the title compound.

LC-MS (Method 1): Rₜ=0.55 min; MS (ESIpos): m/z=296 [M+H]⁺

### Intermediate 456

### {6-amino-5-[(1R)-1-phenylethoxy]pyridin-3-yl}boronic acid

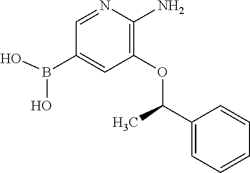 [SMILES: C[C@@H](Oc1cc(B(O)O)cnc1N)c1ccccc1]

5-Bromo-3-[(1R)-1-phenylethoxy]pyridin-2-amine (219 mg, 748 μmol), 4,4,4′,4′,5,5,5′,5′-octamethyl-2,2′-bi-1,3,2-dioxaborolane (228 mg, 897 μmol) and potassium acetate (220 mg, 2.24 mmol) were suspended in degassed 1,4-dioxane (7 mL) under nitrogen. Pd(dppf)Cl₂ (52.5 mg, 74.8 μmol) was added and the mixture was stirred for 2 h at 100° C. The reaction mixture was allowed to cool down, diluted with ethyl acetate and water. The aqueous phase was extracted 3 times with ethyl acetate. The combined organic layers were dried over a hydrophobic filter and concentrated to give 396.9 mg (45% purity, 92% yield) of the title compound.

LC-MS (Method 1): Rₜ=0.71 min; MS (ESIpos): m/z=259 [M+H]⁺

### Intermediate 457

### (rac)-{6-amino-5-[1-(1-methyl-1H-pyrazol-5-yl)ethoxy]pyridin-3-yl}boronic acid

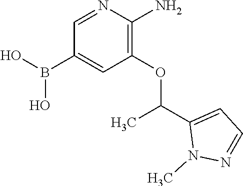 [SMILES: CC(Oc1cc(B(O)O)cnc1N)c1ccnn1C]

(Rac)-5-Bromo-3-[1-(1-methyl-1H-pyrazol-5-yl)ethoxy]pyridin-2-amine (315 mg, 1.06 mmol), 4,4,4′,4′,5,5,5′,5′-octamethyl-2,2′-bi-1,3,2-dioxaborolane (323 mg, 1.27 mmol) and potassium acetate (312 mg, 3.18 mmol) were suspended in degassed 1,4-dioxane (7 mL). Pd(dppf)Cl₂ (38.8 mg, 53.0 μmol) was added and the mixture was stirred for 2 h at 100° C. The reaction mixture was allowed to cool down, diluted with ethyl acetate and filtered. The filtrate was washed with water and brine, dried over sodium sulfate, filtered and concentrated to give 454 mg (45% purity, 73% yield) of the title compound.

LC-MS (Method 1): Rₜ=0.54 min; MS (ESIpos): m/z=263 [M+H]⁺

### Intermediate 458

### {6-amino-5-[(1R)-1-(2-fluorophenyl)ethoxy]pyridin-3-yl}boronic acid

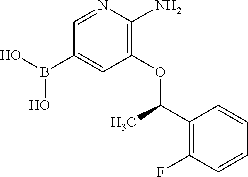 [SMILES: C[C@@H](Oc1cc(B(O)O)cnc1N)c1ccccc1F]

5-Bromo-3-[(1R)-1-(2-fluorophenyl)ethoxy]pyridin-2-amine (457 mg, 85% purity, 1.25 mmol), 4,4,4′,4′,5,5,5′,5′-octamethyl-2,2′-bi-1,3,2-dioxaborolane (380 mg, 1.50 mmol) and potassium acetate (368 mg, 3.75 mmol) were suspended in degassed 1,4-dioxane (5.9 mL). Pd(dppf)Cl₂ (38.8 mg, 53.0 μmol) was added and the mixture was stirred for 2 h at 100° C. The reaction mixture was allowed to cool down, diluted with ethyl acetate and filtered. The filtrate was washed with water and brine, dried over sodium sulfate, filtered and concentrated to give 825 mg (25% purity, 60% yield) of the title compound.

LC-MS (Method 1): Rₜ=0.75 min; MS (ESIpos): m/z=277 [M+H]⁺

### Intermediate 459

### (6-amino-5-{methyl[(1R)-1-phenylethyl]amino}pyridin-3-yl)boronic acid

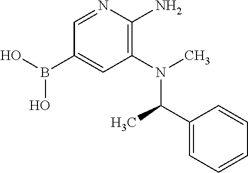 [SMILES: C[C@H](c1ccccc1)N(C)c1cc(B(O)O)cnc1N]

5-Bromo-N³-methyl-N³-[(1R)-1-phenylethyl]pyridine-2,3-diamine (100 mg, 90% purity, 294 μmol), 4,4,4′,4′,5,5,5′,5′-octamethyl-2,2′-bi-1,3,2-dioxaborolane (89.6 mg, 353 μmol) and potassium acetate (86.5 mg, 882 μmol) were suspended in 1,4-dioxane (2.1 mL) and degassed. Pd(dppf)Cl₂ (10.8 mg, 14.7 μmol) was added and the mixture was stirred for 2 h at 100° C. The reaction mixture was allowed to cool down, diluted with ethyl acetate and filtered. The filtrate was washed with water and brine, dried over sodium sulfate, filtered and concentrated to give 258 mg (17% purity, 55% yield) of the title compound.

LC-MS (Method 1): Rₜ=0.85 min; MS (ESIpos): m/z=272 [M+H]⁺

### Intermediate 460

### {6-amino-5-[(1R)-1-(4-methylpyridin-2-yl)ethoxy]pyridin-3-yl}boronic acid

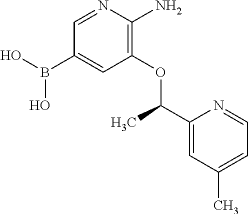 [SMILES: Cc1ccnc([C@@H](C)Oc2cc(B(O)O)cnc2N)c1]

5-Bromo-3-[(1R)-1-(4-methylpyridin-2-yl)ethoxy]pyridin-2-amine (183 mg, 594 μmol), 4,4,4′,4′,5,5,5′,5′-octamethyl-2,2′-bi-1,3,2-dioxaborolane (181 mg, 713 μmol) and potassium acetate (175 mg, 1.78 μmol) were suspended in 1,4-dioxane (2.8 mL) and degassed. Pd(dppf)Cl₂ (20.8 mg, 29.7 μmol) was added and the mixture was stirred for 2 h at 100° C. The reaction mixture was allowed to cool down, diluted with ethyl acetate and filtered. The filtrate was washed with water and brine, dried over sodium sulfate, filtered and concentrated to give 375 mg (34% purity, 78% yield) of the title compound.

LC-MS (Method 1): Rₜ=0.62 min; MS (ESIpos): m/z=274 [M+H]⁺

### Intermediate 461

### {6-amino-5-[(1R)-1-(6-methylpyridin-2-yl)ethoxy]pyridin-3-yl}boronic acid

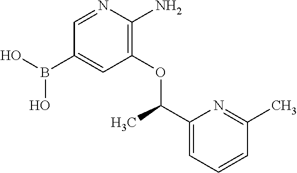 [SMILES: Cc1cccc([C@@H](C)Oc2cc(B(O)O)cnc2N)n1]

5-Bromo-3-[(1R)-1-(6-methylpyridin-2-yl)ethoxy]pyridin-2-amine (120 mg, 389 μmol), 4,4,4′,4′,5,5,5′,5′-octamethyl-2,2′-bi-1,3,2-dioxaborolane (119 mg, 467 μmol) and potassium acetate (115 mg, 1.17 mmol) were suspended in 1,4-dioxane (1.8 mL) and degassed. Pd(dppf)Cl₂ (13.7 mg, 19.5 μmol) was added and the mixture was stirred for 2 h at 100° C. The reaction mixture was allowed to cool down, diluted with ethyl acetate and filtered. The filtrate was washed with water and brine, dried over sodium sulfate, filtered and concentrated to give 219 mg (33% purity, 68% yield) of the title compound.

LC-MS (Method 1): Rₜ=0.59 min; MS (ESIpos): m/z=274 [M+H]⁺

### Intermediate 462

### (rac)-3-[1-(1,3-oxazol-2-yl)ethoxy]-5-(4,4,5,5-tetramethyl-1,3,2-dioxaborolan-2-yl)pyridin-2-amine

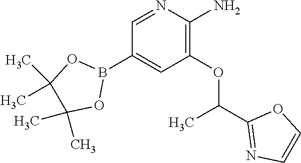 [SMILES: CC(Oc1cc(B2OC(C)(C)C(C)(C)O2)cnc1N)c1ncco1]

(Rac)-5-Bromo-3-[1-(1,3-oxazol-2-yl)ethoxy]pyridin-2-amine (105 mg, 370 μmol) and 4,4,4′,4′,5,5,5′,5′-octamethyl-2,2′-bi-1,3,2-dioxaborolane (113 mg, 443 μmol) were dissolved in 1,4-dioxane (1.9 mL) and degassed. Potassium acetate (109 mg, 1.11 mmol) and Pd(dppf)Cl₂ (13.5 mg, 18.5 μmol) were added and the mixture was stirred for 3 h at 100° C. The reaction mixture was allowed to cool down, diluted with ethyl acetate and filtered. The filtrate was washed with water and brine, dried over magnesium sulfate, filtered and concentrated to give 182 mg (48% purity, 71% yield) of the title compound.

LC-MS (Method 1): Rₜ=0.81 min; MS (ESIpos): m/z=332 [M+H]⁺

¹H-NMR (400 MHz, DMSO-d₆): δ [ppm]=1.25 (s, 12H), 1.66 (d, 3H), 5.56-5.63 (m, 1H), 6.14 (br s, 2H), 7.17 (s, 1H), 7.22 (s, 1H), 7.83 (d, 1H), 8.12 (s, 1H).

### Intermediate 463

### {6-amino-5-[(1R)-1-(3-chlorophenyl)ethoxy]pyridin-3-yl}boronic acid

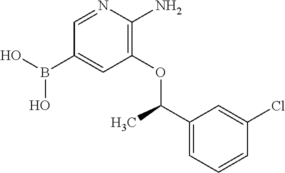 [SMILES: C[C@@H](Oc1cc(B(O)O)cnc1N)c1cccc(Cl)c1]

5-Bromo-3-[(1R)-1-(3-chlorophenyl)ethoxy]pyridin-2-amine (245 mg, 94% purity, 703 μmol), 4,4,4′,4′,5,5,5′,5′-octamethyl-2,2′-bi-1,3,2-dioxaborolane (234 mg, 99% purity, 914 μmol) and potassium acetate (207 mg, 2.11 mmol) were dissolved in 1,4-dioxane (15 mL) under argon. Pd(dppf)Cl₂dichloromethane complex (57.4 mg, 70.3 μmol) was added and stirred for 4 h at 100° C. The reaction mixture was allowed to cool down, concentrated, diluted with ethyl acetate and filtered over celite. The organic phase was washed with water and brine, dried over a hydrophobic filter and concentrated. The residue was purified by preparative HPLC to give 79.9 mg (88% purity, 27% yield) of the title compound.

LC-MS (Method 1): Rₜ=0.86 min; MS (ESIpos): m/z=293 [M+H]⁺

### Intermediate 464

### {6-amino-5-[(1R)-1-(2-methoxyphenyl)ethoxy]pyridin-3-yl}boronic acid

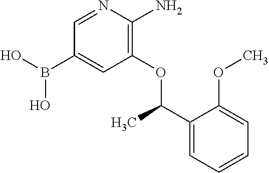 [SMILES: COc1ccccc1[C@@H](C)Oc1cc(B(O)O)cnc1N]

5-Bromo-3-[(1R)-1-(2-methoxyphenyl)ethoxy]pyridin-2-amine (380 mg, 1.18 mmol), 4,4,4′,4′,5,5,5′,5′-octamethyl-2,2′-bi-1,3,2-dioxaborolane (392 mg, 99% purity, 1.53 mmol) and potassium acetate (346 mg, 3.53 mmol) were dissolved in 1,4-dioxane (25 mL) under nitrogen. Pd(dppf)Cl₂ dichloromethane complex (96.0 mg, 118 μmol) was added and the mixture was stirred for 23 h at 100° C. The reaction mixture was allowed to cool down, concentrated, diluted with ethyl acetate and filtered over celite. The organic phase was washed with water and brine, dried over a hydrophobic filter and concentrated. The residue was purified by preparative HPLC to give 142 mg (63% purity, 20% yield) of the title compound.

LC-MS (Method 1): Rₜ=0.81 min; MS (ESIpos): m/z=289 [M+H]⁺

### Intermediate 465

### {6-amino-5-[(1R)-1-(pyridin-3-yl)ethoxy]pyridin-3-yl}boronic acid

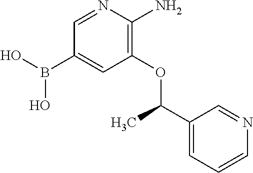 [SMILES: C[C@@H](Oc1cc(B(O)O)cnc1N)c1cccnc1]

5-Bromo-3-[(1R)-1-(pyridin-3-yl)ethoxy]pyridin-2-amine (120 mg, 408 μmol), 4,4,4′,4′,5,5,5′,5′-octamethyl-2,2′-bi-1,3,2-dioxaborolane (124 mg, 490 μmol) and potassium acetate (120 mg, 1.22 mmol) were dissolved in degassed 1,4-dioxane (1.9 mL). Pd(dppf)Cl₂ (14.3 mg, 20.4 μmol) was added and the mixture was stirred for 3 h at 100° C. The reaction mixture was allowed to cool down, diluted with ethyl acetate and filtered. The filtrate was washed with water and brine, dried and concentrated to give 252 mg (13% purity, 23% yield) of the title compound.

LC-MS (Method 1): Rₜ=0.48 min; MS (ESIpos): m/z=260 [M+H]⁺

¹H-NMR (400 MHz, DMSO-d₆) 5 [ppm]: 1.066 (3.27), 1.156 (16.00), 1.164 (0.87), 1.173 (0.21), 1.229 (1.93), 1.292 (0.38), 1.541 (0.39), 1.557 (0.39), 1.907 (0.45), 1.988 (0.35), 2.518 (0.61), 2.523 (0.46), 3.566 (9.98), 3.938 (0.52), 6.266 (0.17), 7.043 (0.16), 7.046 (0.16), 7.773 (0.23), 7.776 (0.23), 7.939 (1.14).

### Intermediate 466

### 5-(4,4,5,5-tetramethyl-1,3,2-dioxaborolan-2-yl)-3-[(1R)-1-(1,3-thiazol-2-yl)ethoxy]pyridin-2-amine

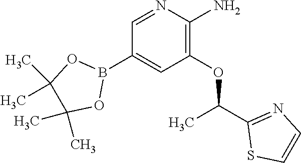 [SMILES: C[C@@H](Oc1cc(B2OC(C)(C)C(C)(C)O2)cnc1N)c1nccs1]

5-Bromo-3-[(1R)-1-(1,3-thiazol-2-yl)ethoxy]pyridin-2-amine (140 mg, 466 μmol) and 4,4,4′,4′,5,5,5′,5′-octamethyl-2,2′-bi-1,3,2-dioxaborolane (142 mg, 560 μmol) were dissolved in degassed 1,4-dioxane (2.4 mL). Potassium acetate (137 mg, 1.40 mmol) and Pd(dppf)Cl₂ (17.1 mg, 23.3 μmol) were added and the mixture was stirred for 3 h at 100° C. The reaction mixture was allowed to cool down, diluted with ethyl acetate and filtered. The filtrate was washed with water and brine, dried over magnesium sulfate, filtered and concentrated to give 254 mg (49% purity, 76% yield) of the title compound.

¹H-NMR (400 MHz, DMSO-d₆): δ [ppm]=1.23 (s, 12H), 1.68 (d, 3H), 5.82 (q, 1H), 6.22 (s, 2H), 7.11 (d, 1H), 7.71 (d, 1H), 7.79 (d, 1H), 7.82 (d, 1H).

### Intermediate 467

### 3-[(1R)-1-(3-fluorophenyl)ethoxy]-5-(4,4,5,5-tetramethyl-1,3,2-dioxaborolan-2-yl)pyridin-2-amine

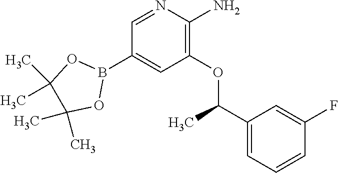 [SMILES: C[C@@H](Oc1cc(B2OC(C)(C)C(C)(C)O2)cnc1N)c1cccc(F)c1]

5-Bromo-3-[(1R)-1-(3-fluorophenyl)ethoxy]pyridin-2-amine (265 mg, 97% purity, 826 μmol), 4,4,4′,4′,5,5,5′,5′-octamethyl-2,2′-bi-1,3,2-dioxaborolane (275 mg, 99% purity, 1.07 mmol) and potassium acetate (243 mg, 2.48 mmol) were dissolved in 1,4-dioxane (25 mL) under argon. Pd(dppf)Cl₂ (60.4 mg, 82.6 μmol) was added and the mixture was stirred for 3 h at 100° C. The reaction mixture was allowed to cool down, concentrated, diluted with ethyl acetate and filtered over celite. The organic phase was washed with water and brine, dried over a hydrophobic filter and concentrated. The crude product was purified by preparative HPLC to give 104 mg (71% purity, 25% yield) of the title compound.

### Intermediate 468

### 3-[1-(1,3-oxazol-4-yl)ethoxy]-5-(4,4,5,5-tetramethyl-1,3,2-dioxaborolan-2-yl)pyridin-2-amine (Enantiomer 1 and enantiomer 2)

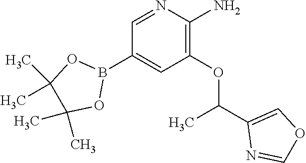 [SMILES: CC(Oc1cc(B2OC(C)(C)C(C)(C)O2)cnc1N)c1cocn1]

5-Bromo-3-[1-(1,3-oxazol-4-yl)ethoxy]pyridin-2-amine (140 mg, 493 μmol) and 4,4,4′,4′,5,5,5′,5′-octamethyl-2,2′-bi-1,3,2-dioxaborolane (150 mg, 591 μmol) were dissolved in 1,4-dioxane (2.5 mL) and degassed. Potassium acetate (145 mg, 1.48 mmol) and Pd(dppf)Cl₂ (36.1 mg, 49.3 μmol) were added and the mixture was stirred for 3 h at 100° C. The reaction mixture was allowed to cool down, diluted with ethyl acetate and filtered. The filtrate was washed with water and brine, dried over magnesium sulfate, filtered and concentrated to give 279 mg (51% purity, 87% yield) of the title compound.

LC-MS (Method 1): Rₜ=0.78 min; MS (ESIpos): m/z=332 [M+H]⁺

¹H-NMR (400 MHz, DMSO-d₆): δ [ppm]=1.25 (s, 12H), 1.56 (d, 3H), 5.39 (q, 1H), 6.12 (br s, 2H), 7.15 (s, 1H), 7.83 (s, 1H), 8.13 (s, 1H), 8.38 (d, 1H).

### Intermediate 469

### 3-[(1R)-1-(pyrimidin-2-yl)ethoxy]-5-(4,4,5,5-tetramethyl-1,3,2-dioxaborolan-2-yl)pyridin-2-amine

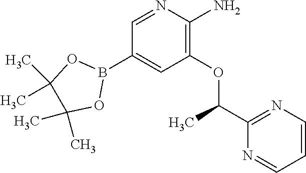 [SMILES: C[C@@H](Oc1cc(B2OC(C)(C)C(C)(C)O2)cnc1N)c1ncccn1]

5-Bromo-3-[(1R)-1-(pyrimidin-2-yl)ethoxy]pyridin-2-amine (100 mg, 339 μmol) was dissolved in degassed 1,4-dioxane (3 mL). 4,4,4′,4′,5,5,5′,5′-octamethyl-2,2′-bi-1,3,2-dioxaborolane (103 mg, 407 μmol), potassium acetate (99.7 mg, 1.0 mmol) and Pd(dppf)Cl₂ (24.7 mg, 34 μmol) were added and the mixture was stirred over night at 100° C. The reaction mixture was allowed to cool down, filtered and used without purification in the next step.

### Intermediate 470

### 3-[(1R)-1-(pyridin-3-yl)ethoxy]-5-(4,4,5,5-tetramethyl-1,3,2-dioxaborolan-2-yl)pyridin-2-amine

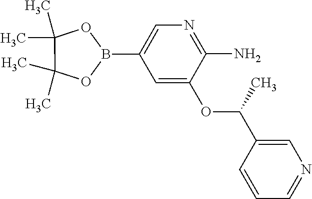 [SMILES: C[C@@H](Oc1cc(B2OC(C)(C)C(C)(C)O2)cnc1N)c1cccnc1]

5-Bromo-3-[(1R)-1-(pyridin-3-yl)ethoxy]pyridin-2-amine (940 mg, 3.20 mmol) and 4,4,4′,4′,5,5,5′,5′-octamethyl-2,2′-bi-1,3,2-dioxaborolane (893 mg, 3.52 mmol) were dissolved in 1,4-dioxane (28 mL) under argon. Potassium acetate (941 mg, 9.59 mmol) and Pd(dppf)Cl₂ complex with dichloromethane (261 mg, 320 μmol) were added and the mixture was stirred for 4.5 h at 100° C. The reaction mixture was allowed to cool down, diluted with dichloromethane, filtered and concentrated to give 2.05 g (33% purity, 62% yield) of the title compound.

¹H-NMR (400 MHz, DMSO-d6) δ[ppm]: 1.065 (0.48), 1.155 (16.00), 1.228 (4.79), 1.256 (0.51), 1.291 (0.68), 1.541 (0.73), 1.556 (0.74), 1.900 (0.50), 3.565 (6.87), 6.267 (0.47), 7.044 (0.41), 7.774 (0.41), 7.945 (0.45).

### Intermediate 471

### 5-(4,4,5,5-tetramethyl-1,3,2-dioxaborolan-2-yl)-3-[(1R)-1-(1,2-thiazol-5-yl)ethoxy]pyridin-2-amine

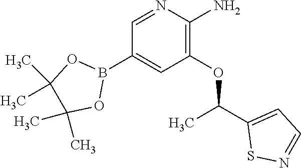 [SMILES: C[C@@H](Oc1cc(B2OC(C)(C)C(C)(C)O2)cnc1N)c1ccns1]

5-Bromo-3-[(1R)-1-(1,2-thiazol-5-yl)ethoxy]pyridin-2-amine (123 mg, 410 μmol) and 4,4,4′,4′,5,5,5′,5′-octamethyl-2,2′-bi-1,3,2-dioxaborolane (125 mg, 492 μmol) were dissolved in degassed 1,4-dioxane (2.1 mL). Potassium acetate (121 mg, 1.23 mmol) and Pd(dppf)Cl₂ (15.0 mg, 20.5 μmol) were added and the mixture was stirred for 4.5 h at 100° C. The reaction mixture was allowed to cool down, diluted with ethyl acetate and filtered. The filtrate was washed with water and brine, dried over magnesium sulfate, filtered and concentrated to give 197 mg (62% purity, 86% yield) of the title compound.

LC-MS (Method 1): Rₜ=0.91 min; MS (ESIpos): m/z=348 [M+H]⁺

¹H-NMR (400 MHz, DMSO-d₆): δ [ppm]=1.24 (s, 12H), 1.64 (d, 3H), 6.06 (q, 1H), 6.22 (s, 2H), 7.13 (s, 1H), 7.43 (d, 1H), 7.83 (d, 1H), 8.49 (d, 1H).

### Intermediate 472

### (rac)-3-[1-(2,6-dichlorophenyl)ethoxy]-5-(4,4,5,5-tetramethyl-1,3,2-dioxaborolan-2-yl)pyridin-2-amine

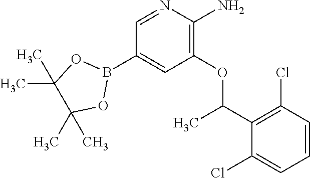 [SMILES: CC(Oc1cc(B2OC(C)(C)C(C)(C)O2)cnc1N)c1c(Cl)cccc1Cl]

(Rac)-5-Bromo-3-[1-(2,6-dichlorophenyl)ethoxy]pyridin-2-amine (337 mg, 931 μmol), 4,4,4′,4′,5,5,5′,5′-octamethyl-2,2′-bi-1,3,2-dioxaborolane (260 mg, 1.02 mmol) and potassium acetate (274 mg, 2.7 mmol) were dissolved in degassed 1,4-dioxane (9 mL) under argon. Pd(dppf)Cl₂ (38.0 mg, 46.5 μmol) was added and the mixture was stirred for 3 h at 100° C. The reaction mixture was allowed to cool down, filtered and the filtrate was used in the next step without further work-up or purification.

### Intermediate 473

### (rac)-4-[3-(1-{[2-amino-5-(4,4,5,5-tetramethyl-1,3,2-dioxaborolan-2-yl)pyridin-3-yl]oxy}ethyl)phenyl]-2-methylbut-3-yn-2-ol

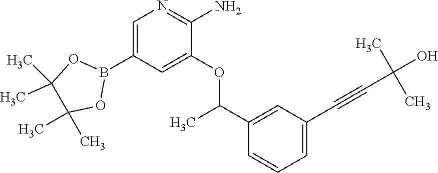 [SMILES: CC(Oc1cc(B2OC(C)(C)C(C)(C)O2)cnc1N)c1cccc(C#CC(C)(C)O)c1]

(Rac)-4-(3-{1-[(2-Amino-5-bromopyridin-3-yl)oxy]ethyl}phenyl)-2-methylbut-3-yn-2-ol (155 mg, 413 μmol) was dissolved in degassed 1,4-dioxane (3.7 mL). 4,4,4′,4′,5,5,5′,5′-Octamethyl-2,2′-bi-1,3,2-dioxaborolane (126 mg, 496 μmol), potassium acetate (121.6 mg, 1.2 mmol) and Pd(dppf)Cl₂ (30.2 mg, 41 μmol) were added and the mixture was stirred over night at 100° C. The reaction mixture was allowed to cool down, filtered and the filtrate was used in the next step without further work-up or purification

### Intermediate 474

### 3-[(6,7-dihydro-5H-cyclopenta[b]pyridin-7-yl)oxy]-5-(4,4,5,5-tetramethyl-1,3,2-dioxaborolan-2-yl)pyridin-2-amine (Enantiomer 2)

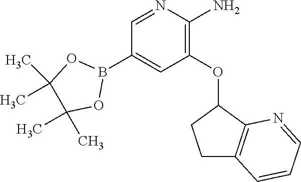 [SMILES: CC1(C)OB(c2cnc(N)c(OC3CCc4cccnc43)c2)OC1(C)C]

5-Bromo-3-[(6,7-dihydro-5H-cyclopenta[b]pyridin-7-yl)oxy]pyridin-2-amine (enantiomer 2) (120 mg, 392 μmol), 4,4,4′,4′,5,5,5′,5′-octamethyl-2,2′-bi-1,3,2-dioxaborolane (119 mg, 470 μmol) and potassium acetate (115 mg, 1.18 mmol) were suspended in degassed 1,4-dioxane (2 mL). Pd(dppf)Cl₂ (14.3 mg, 19.6 μmol) was added and the mixture was stirred for 2 h at 100° C. The reaction mixture was allowed to cool down, diluted with ethyl acetate and filtered. The filtrate was washed with water and brine, dried over sodium sulfate, filtered and concentrated to give 223 mg (40% purity, 83% yield) of the title compound.

LC-MS (Method 1): Rₜ=0.82 min; MS (ESIpos): m/z=354 [M+H]⁺

### Intermediate 475

### (rac)-3-[(6,7-dihydro-5H-cyclopenta[b]pyridin-7-yl)oxy]-5-(4,4,5,5-tetramethyl-1,3,2-dioxaborolan-2-yl)pyridin-2-amine

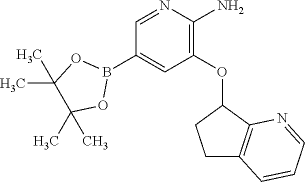 [SMILES: CC1(C)OB(c2cnc(N)c(OC3CCc4cccnc43)c2)OC1(C)C]

(Rac)-5-Bromo-3-[(6,7-dihydro-5H-cyclopenta[b]pyridin-7-yl)oxy]pyridin-2-amine (300 mg, 980 μmol) was dissolved in degassed 1,4-dioxane (8.7 mL). 4,4,4′,4′,5,5,5′,5′-octamethyl-2,2′-bi-1,3,2-dioxaborolane (299 mg, 1.18 mmol), potassium acetate (288 mg, 2.94 mmol) and Pd(dppf)Cl₂ (71.7 mg, 98.0 μmol) were added and the mixture was stirred over night at 100° C. The reaction mixture was allowed to cool down, filtered and the filtrate was used in the next step without further work-up or purification.

### Intermediate 476

### 3-(difluoromethyl)-5-(4,4,5,5-tetramethyl-1,3,2-dioxaborolan-2-yl)pyridin-2-amine

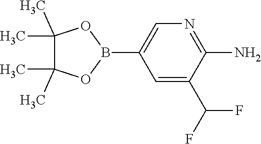 [SMILES: CC1(C)OB(c2cnc(N)c(C(F)F)c2)OC1(C)C]

5-Bromo-3-(difluoromethyl)pyridin-2-amine (350 mg, 1.57 mmol) and 4,4,4′,4′,5,5,5′,5′-octamethyl-2,2′-bi-1,3,2-dioxaborolane (438 mg, 1.73 mmol) were dissolved in 1,4-dioxane (14 mL). Potassium acetate (462 mg, 4.71 mmol) was added and the mixture was degassed. Pd(dppf)Cl₂ (64.1 mg, 78.5 μmol) was added and stirred for 2 h at 100° C. The reaction mixture was allowed to cool down, diluted with water and extracted with ethyl acetate. The combined organic layers were washed with brine, dried over a hydrophobic filter and concentrated to give 970 mg (43% purity, 98% yield) of the title compound.

LC-MS (Method 1): Rₜ=0.72 min; MS (ESIpos): m/z=271.5 [M+H]⁺

### Intermediate 477

### 5-(4,4,5,5-tetramethyl-1,3,2-dioxaborolan-2-yl)-3-(trifluoromethyl)pyridin-2-amine

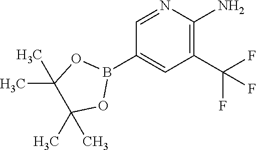 [SMILES: CC1(C)OB(c2cnc(N)c(C(F)(F)F)c2)OC1(C)C]

5-Bromo-3-(trifluoromethyl)pyridin-2-amine (5.40 g, 22.4 mmol), 4,4,4′,4′,5,5,5′,5′-octamethyl-2,2′-bi-1,3,2-dioxaborolane (6.26 g, 24.6 mmol) and potassium acetate (6.6 g, 67.2 mmol) were dissolved in dry 1,4-dioxane (230 mL). The mixture was degassed and Pd(dppf)Cl₂ (915 mg, 1.12 mmol) was added and stirred for 3 h at 100° C. The reaction mixture was allowed to cool down, diluted with water and extracted with ethyl acetate. The combined organic layers were dried over sodium sulfate, filtered and concentrated to give 7.30 g (113% yield) of the title compound.

LC-MS (Method 1): Rₜ=0.76 min; MS (ESIpos): m/z=289 [M+H]⁺

¹H-NMR (400 MHz, DMSO-d6) δ[ppm]: 1.065 (0.73), 1.154 (16.00), 1.163 (0.81), 1.269 (14.15), 1.284 (0.53), 1.291 (0.84), 6.922 (0.52), 7.800 (0.62), 7.802 (0.63), 8.384 (0.58).

### Intermediate 478

### 3-[(1R)-1-(2,6-dichloro-3-fluorophenyl)ethoxy]-5-(4,4,5,5-tetramethyl-1,3,2-dioxaborolan-2-yl)pyridin-2-amine

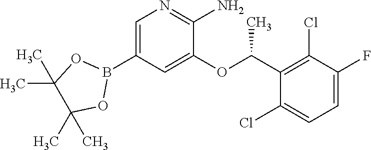 [SMILES: C[C@@H](Oc1cc(B2OC(C)(C)C(C)(C)O2)cnc1N)c1c(Cl)ccc(F)c1Cl]

5-Bromo-3-[(1R)-1-(2,6-dichloro-3-fluorophenyl)ethoxy]pyridin-2-amine (500 mg, 1.32 mmol), 4,4,4′,4′,5,5,5′,5′-octamethyl-2,2′-bi-1,3,2-dioxaborolane (368 mg, 1.45 mmol) and potassium acetate (387 mg, 3.9 mmol) were dissolved in degassed 1,4-dioxane (13 mL). Pd(dppf)Cl₂ (53.7 mg, 65.8 μmol) was added and the mixture was stirred for 5 h at 100° C. The reaction mixture was allowed to cool down, filtered and the filtrate was used without further purification in the next step.

LC-MS (Method 1): Rₜ=1.38 min; MS (ESIpos): m/z=427 [M+H]⁺

### Intermediate 479

### (rac)-3-[1-(4,5-dimethyl-1,3-thiazol-2-yl)ethoxy]-5-(4,4,5,5-tetramethyl-1,3,2-dioxaborolan-2-yl)pyridin-2-amine

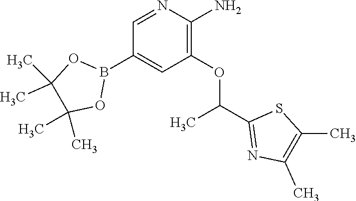

(Rac)-5-Bromo-3-[1-(4,5-dimethyl-1,3-thiazol-2-yl)ethoxy]pyridin-2-amine (186 mg, 567 μmol) and 4,4,4′,4′,5,5,5′,5′-octamethyl-2,2′-bi-1,3,2-dioxaborolane (187 mg, 737 μmol) were dissolved in degassed 1,4-dioxane (2.9 mL) under argon. Potassium acetate (167 mg, 1.70 mmol) and Pd(dppf)Cl₂ (41.5 mg, 56.7 μmol) were added and the mixture was stirred for 3 h at 100° C. The reaction mixture was allowed to cool down, diluted with ethyl acetate and filtered. The filtrate was washed with water and brine, dried over magnesium sulfate, filtered and concentrated to give 356 mg (39% purity, 65% yield) of the title compound.

LC-MS (Method 1): Rₜ=1.10 min; MS (ESIpos): m/z=376 [M+H]⁺

¹H-NMR (400 MHz, DMSO-d6) δ[ppm]: 1.065 (1.42), 1.156 (16.00), 1.163 (8.28), 1.239 (6.27), 1.292 (0.71), 1.621 (0.99), 1.628 (0.49), 1.637 (1.00), 2.228 (2.16), 2.281 (2.00), 3.565 (3.37), 6.180 (0.47), 7.123 (0.51), 7.802 (0.52), 7.805 (0.52), 7.943 (1.34).

### Intermediate 480

### 3-[(1R)-1-(pyridin-4-yl)ethoxy]-5-(4,4,5,5-tetramethyl-1,3,2-dioxaborolan-2-yl)pyridin-2-amine

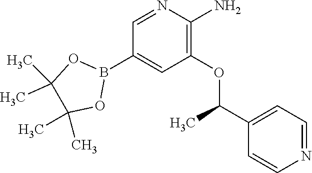 [SMILES: C[C@@H](Oc1cc(B2OC(C)(C)C(C)(C)O2)cnc1N)c1ccncc1]

5-Bromo-3-[(1R)-1-(pyridin-4-yl)ethoxy]pyridin-2-amine (489 mg, 75% purity, 1.25 mmol), 4,4,4′,4′,5,5,5′,5′-octamethyl-2,2′-bi-1,3,2-dioxaborolane (380 mg, 1.50 mmol) and potassium acetate (367 mg, 3.74 mmol) were dissolved in 1,4-dioxane (5.9 mL) under argon. Pd(dppf)Cl₂ (43.8 mg, 62.3 μmol; CAS-RN:[13965-03-2]) was added and the mixture was stirred for 1 h at 100° C. 4,4,4′,4′,5,5,5′,5′-octamethyl-2,2′-bi-1,3,2-dioxaborolane (100 mg, 0.39 mmol) was added again and stirred for 1 h at 100° C. The reaction mixture was allowed to cool down, diluted with ethyl acetate and filtered. The filtrate was washed with water and brine, dried and concentrated to give 833 mg (26% purity, 51% yield) of the title compound.

LC-MS (Method 1): Rₜ=0.74 min; MS (ESIpos): m/z=342 [M+H]⁺

¹H-NMR (400 MHz, DMSO-d6) δ[ppm]: 1.066 (8.27), 1.156 (16.00), 1.164 (9.04), 1.171 (0.46), 1.189 (0.18), 1.217 (1.73), 1.219 (1.75), 1.232 (0.33), 1.239 (0.24), 1.274 (0.51), 1.292 (0.27), 1.511 (0.46), 1.527 (0.46), 1.542 (0.32), 1.558 (0.31), 1.907 (0.27), 1.987 (0.40), 2.518 (0.28), 2.522 (0.19), 3.330 (3.03), 3.937 (1.12), 6.300 (0.20), 6.963 (0.19), 6.965 (0.19), 7.468 (0.24), 7.471 (0.17), 7.479 (0.17), 7.483 (0.24), 7.782 (0.22), 7.785 (0.23), 7.940 (1.02), 8.532 (0.30), 8.536 (0.26), 8.547 (0.22).

### Intermediate 481

### (rac)-3-[1-(5-methyl-1,3-thiazol-2-yl)ethoxy]-5-(4,4,5,5-tetramethyl-1,3,2-dioxaborolan-2-yl)pyridin-2-amine

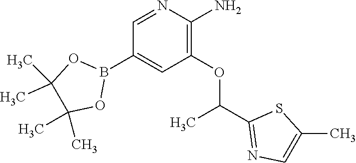

(Rac)-5-Bromo-3-[1-(5-methyl-1,3-thiazol-2-yl)ethoxy]pyridin-2-amine (204 mg, 649 μmol) and 4,4,4′,4′,5,5,5′,5′-octamethyl-2,2′-bi-1,3,2-dioxaborolane (214 mg, 844 μmol) were dissolved in degassed 1,4-dioxane (3.3 mL). Potassium acetate (191 mg, 1.95 mmol) and Pd(dppf)Cl₂ (47.5 mg, 64.9 μmol) were added and the mixture was stirred for 3 h at 100° C. The reaction mixture was allowed to cool down, diluted with ethyl acetate and filtered. The filtrate was washed with water and brine, dried over magnesium sulfate, filtered and concentrated to give 335 mg (38% purity, 54% yield) of the title compound which was used in the next step without further purification.

LC-MS (Method 1): Rₜ=0.97 min; MS (ESIpos): m/z=362 [M+H]⁺

¹H-NMR (400 MHz, DMSO-d6) δ[ppm]: 1.066 (1.69), 1.156 (16.00), 1.164 (1.41), 1.237 (3.24), 1.637 (0.51), 1.652 (0.73), 2.394 (0.85), 2.397 (1.09), 2.401 (0.50), 3.566 (4.65), 7.943 (1.40).

### Intermediate 482

### (rac)-5-(4,4,5,5-tetramethyl-1,3,2-dioxaborolan-2-yl)-3-[1-(1,3-thiazol-4-yl)ethoxy]pyridin-2-amine

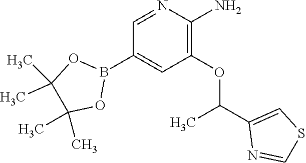 [SMILES: CC(Oc1cc(B2OC(C)(C)C(C)(C)O2)cnc1N)c1cscn1]

(Rac)-5-Bromo-3-[1-(1,3-thiazol-4-yl)ethoxy]pyridin-2-amine (180 mg, 600 μmol) and 4,4,4′,4′,5,5,5′,5′-octamethyl-2,2′-bi-1,3,2-dioxaborolane (183 mg, 720 μmol) were dissolved in degassed 1,4-dioxane (3.1 mL). Potassium acetate (177 mg, 1.80 mmol) and Pd(dppf)Cl₂ (21.9 mg, 30.0 μmol) were added and the mixture was stirred for 1.5 h at 65° C. and 2.5 h at 100° C. The reaction mixture was allowed to cool down, diluted with ethyl acetate and filtered. The filtrate was washed with water and brine, dried over magnesium sulfate, filtered and concentrated to give 288 mg (36% purity, 50% yield) of the title compound which was used without further purification in the next step.

LC-MS (Method 1): Rₜ=0.88 min; MS (ESIpos): m/z=348 [M+H]⁺

¹H-NMR (400 MHz, DMSO-d₆): δ [ppm]=1.24 (s, 12H), 1.61 (d, 3H), 5.57-5.65 (m, 1H), 6.19 (s, 2H), 7.08 (d, 1H), 7.68 (d, 1H), 7.81 (d, 1H), 9.10 (d, 1H).

### Intermediate 483

### 3-[(1R)-1-(5-methyl-1,3,4-oxadiazol-2-yl)ethoxy]-5-(4,4,5,5-tetramethyl-1,3,2-dioxaborolan-2-yl)pyridin-2-amine

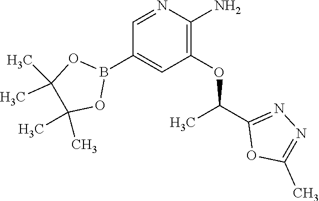 [SMILES: Cc1nnc([C@@H](C)Oc2cc(B3OC(C)(C)C(C)(C)O3)cnc2N)o1]

5-Bromo-3-[(1R)-1-(5-methyl-1,3,4-oxadiazol-2-yl)ethoxy]pyridin-2-amine (100 mg, 334 μmol) and 4,4,4′,4′,5,5,5′,5′-octamethyl-2,2′-bi-1,3,2-dioxaborolane (102 mg, 401 μmol) were dissolved in degassed 1,4-dioxane (1.7 mL). Potassium acetate (98.4 mg, 1.00 mmol) and Pd(dppf)Cl₂ (12.2 mg, 16.7 μmol) were added and the mixture was stirred for 3 h at 100° C. The reaction mixture was allowed to react, diluted with ethyl acetate and filtered. The filtrate was washed with water and brine, dried over magnesium sulfate, filtered and concentrated to give 100 mg (86% yield) which was used without further purification in the next step.

### Intermediate 484

### 3-[(1R)-1-(2-methyl-1,3-thiazol-4-yl)ethoxy]-5-(4,4,5,5-tetramethyl-1,3,2-dioxaborolan-2-yl)pyridin-2-amine

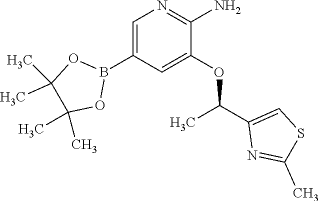 [SMILES: Cc1nc([C@@H](C)Oc2cc(B3OC(C)(C)C(C)(C)O3)cnc2N)cs1]

5-Bromo-3-[(1R)-1-(2-methyl-1,3-thiazol-4-yl)ethoxy]pyridin-2-amine (158 mg, 503 μmol) and 4,4,4′,4′,5,5,5′,5′-octamethyl-2,2′-bi-1,3,2-dioxaborolane (166 mg, 653 μmol) were dissolved in degassed 1,4-dioxane (4.5 mL). Potassium acetate (148 mg, 1.51 mmol) and Pd(dppf)Cl₂ (36.8 mg, 50.3 μmol) were added and the mixture was stirred for 2 h at 100° C. The reaction mixture was allowed to cool down, diluted with ethyl acetate and filtered. The filtrate was washed with water and brine, dried over a hydrophobic filter and concentrated to give 304 mg (40% purity, 67% yield) of the title compound.

LC-MS (Method 1): Rₜ=1.00 min; MS (ESIpos): m/z=362 [M+H]⁺

¹H-NMR (400 MHz, DMSO-d6) δ[ppm]: 1.065 (3.58), 1.156 (16.00), 1.164 (6.12), 1.242 (3.70), 1.574 (0.70), 1.590 (0.71), 2.518 (0.69), 2.523 (0.47), 2.645 (2.18), 3.565 (5.87), 3.940 (0.58), 7.416 (0.53), 7.942 (1.26).

### Intermediate 485

### (rac)-3-[1-(4-methyl-1,3-thiazol-2-yl)ethoxy]-5-(4,4,5,5-tetramethyl-1,3,2-dioxaborolan-2-yl)pyridin-2-amine

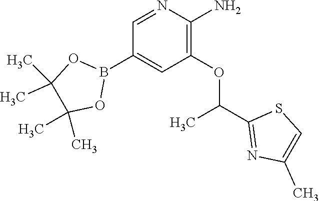 [SMILES: Cc1csc(C(C)Oc2cc(B3OC(C)(C)C(C)(C)O3)cnc2N)n1]

(Rac)-5-Bromo-3-[1-(4-methyl-1,3-thiazol-2-yl)ethoxy]pyridin-2-amine (126 mg, 401 μmol) and 4,4,4′,4′,5,5,5′,5′-octamethyl-2,2′-bi-1,3,2-dioxaborolane (132 mg, 521 μmol) were dissolved in degassed 1,4-dioxane (2.1 mL). Potassium acetate (118 mg, 1.20 mmol) and Pd(dppf)Cl₂ (29.3 mg, 40.1 μmol) were added and the mixture was stirred for 3 h at 100° C. The reaction mixture was allowed to reach rt, diluted with ethyl acetate and filtered. The filtrate was washed with water and brine, dried over a magnesium sulfate, filtered and concentrated to give 222 mg (44% purity, 67% yield) of the title compound.

¹H-NMR (400 MHz, DMSO-d6) δ[ppm]: 1.066 (2.89), 1.156 (16.00), 1.164 (8.17), 1.237 (5.44), 1.292 (0.92), 1.648 (1.18), 1.664 (1.18), 2.347 (2.41), 2.349 (2.47), 2.518 (0.63), 2.523 (0.43), 3.566 (7.03), 3.938 (0.49), 6.203 (0.43), 7.117 (0.50), 7.120 (0.50), 7.230 (0.63), 7.232 (0.66), 7.811 (0.59), 7.814 (0.57), 7.940 (1.36).

### Intermediate 486

### (rac)-3-[1-(1-methyl-1H-1,2,3-triazol-4-yl)ethoxy]-5-(4,4,5,5-tetramethyl-1,3,2-dioxaborolan-2-yl)pyridin-2-amine

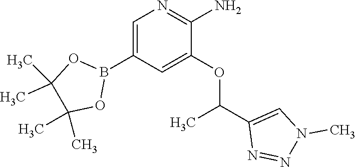

(Rac)-5-Bromo-3-[1-(1-methyl-1H-1,2,3-triazol-4-yl)ethoxy]pyridin-2-amine (281 mg, 941 μmol) and 4,4,4′,4′,5,5,5′,5′-octamethyl-2,2′-bi-1,3,2-dioxaborolane (311 mg, 1.22 mmol) were dissolved in degassed 1,4-dioxane (8.4 mL). Potassium acetate ((277 mg, 2.82 mmol) and Pd(dppf)Cl₂ (68.9 mg, 94.1 μmol) were added and the mixture was stirred for 2 h at 100° C. The reaction mixture was allowed to cool down, diluted with ethyl acetate and filtered. The filtrate was concentrated to give 701 mg (24% purity, 52% yield) of the title compound.

LC-MS (Method 1): Rₜ=0.71 min; MS (ESIpos): m/z=346 [M+H]⁺

¹H-NMR (400 MHz, DMSO-d₆): δ [ppm]=1.25 (s, 12H), 1.60 (d, 3H), 4.01 (s, 3H), 5.57 (q, 1H), 6.10 (s, 2H), 7.17 (d, 1H), 7.80 (d, 1H), 8.09 (s, 1H).

### Intermediate 487

### (rac)-3-{1-[3-(methanesulfonyl)phenyl]ethoxy}-5-(4,4,5,5-tetramethyl-1,3,2-dioxaborolan-2-yl)pyridin-2-amine

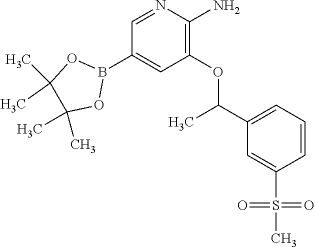 [SMILES: CC(Oc1cc(B2OC(C)(C)C(C)(C)O2)cnc1N)c1cccc(S(C)(=O)=O)c1]

(Rac)-5-Bromo-3-{1-[3-(methanesulfonyl)phenyl]ethoxy}pyridin-2-amine (989 mg, 2.66 mmol), 4,4,4′,4′,5,5,5′,5′-octamethyl-2,2′-bi-1,3,2-dioxaborolane (744 mg, 2.93 mmol) and potassium acetate (784 mg, 7.99 mmol]) were dissolved in degassed 1,4-dioxane (27 mL) under argon. Pd(dppf)Cl₂ (109 mg, 133 μmol) was added and the mixture was stirred over night at 100° C. The reaction mixture was allowed to cool down, filtered and the filtrate was diluted with ethyl acetate and. The organic phase was washed with water. The aqueous phase was extracted with ethyl acetate. The combined organic layers were dried over sodium sulfate, filtered and concentrated to give 1.50 g (135% yield) of the title compound.

LC-MS (Method 1): Rₜ=0.90 min; MS (ESIpos): m/z=419 [M+H]⁺

¹H-NMR (400 MHz, DMSO-d6) δ[ppm]: 1.066 (9.70), 1.156 (15.59), 1.164 (16.00), 1.214 (2.26), 1.222 (2.46), 1.260 (0.80), 1.274 (0.48), 1.292 (1.16), 1.344 (0.51), 1.360 (0.48), 1.550 (0.60), 1.566 (0.60), 1.904 (4.50), 3.203 (1.25), 3.210 (2.09), 3.334 (0.62), 7.766 (0.41), 7.770 (0.44).

### Intermediate 488

### 3-[(1R)-1-(1-methyl-1H-1,2,3-triazol-5-yl)ethoxy]-5-(4,4,5,5-tetramethyl-1,3,2-dioxaborolan-2-yl)pyridin-2-amine

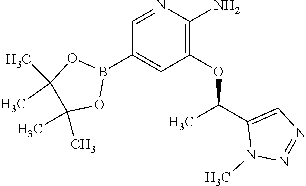 [SMILES: C[C@@H](Oc1cc(B2OC(C)(C)C(C)(C)O2)cnc1N)c1cnnn1C]

5-Bromo-3-[(1R)-1-(1-methyl-1H-1,2,3-triazol-5-yl)ethoxy]pyridin-2-amine (131 mg, 439 μmol) and 4,4,4′,4′,5,5,5′,5′-octamethyl-2,2′-bi-1,3,2-dioxaborolane (134 mg, 527 μmol) were dissolved in degassed 1,4-dioxane (2.3 mL). Potassium acetate (129 mg, 1.32 mmol) and Pd(dppf)Cl₂ (32.1 mg, 43.9 μmol) were added and the mixture was stirred for 4 h at 100° C. The reaction mixture was allowed to cool down, diluted with ethyl acetate and filtered. The filtrate was washed with water and brine, dried over magnesium sulfate, filtered and concentrated to give 226 mg (31% purity, 46% yield) of the title compound.

LC-MS (Method 1): Rₜ=0.67 min; MS (ESIpos): m/z=346 [M+H]⁺

¹H-NMR (400 MHz, DMSO-d₆): δ [ppm]=1.26 (s, 12H), 1.60 (d, 3H), 4.00 (s, 3H), 5.87 (q, 1H), 6.22 (s, 2H), 7.19 (d, 1H), 7.85 (s, 1H), 7.94 (s, 1H).

### Intermediate 489

### (rac)-3-[1-(1-propyl-1H-pyrazol-5-yl)ethoxy]-5-(4,4,5,5-tetramethyl-1,3,2-dioxaborolan-2-yl)pyridin-2-amine

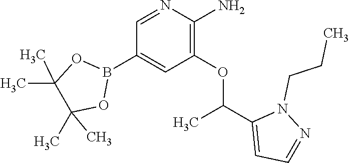 [SMILES: CCCn1nccc1C(C)Oc1cc(B2OC(C)(C)C(C)(C)O2)cnc1N]

(Rac)-5-Bromo-3-[1-(1-propyl-1H-pyrazol-5-yl)ethoxy]pyridin-2-amine (159 mg, 50% purity, 244 μmol), 4,4,4′,4′,5,5,5′,5′-octamethyl-2,2′-bi-1,3,2-dioxaborolane (93.1 mg, 367 μmol) and potassium acetate (96 mg, 978 μmol) were dissolved in degassed 1,4-dioxane (2.5 mL) under argon. Pd(dppf)Cl₂ (9.98 mg, 12.2 μmol) was added and the mixture was stirred for 3 h at 100° C. The reaction mixture was allowed to cool down, filtered and the filtrate was used in the next step without further work-up or purification.

LC-MS (Method 1): Rₜ=1.00 min; MS (ESIpos): m/z=373 [M+H]⁺

### Intermediate 490

### (rac)-3-[1-(pyrimidin-4-yl)ethoxy]-5-(4,4,5,5-tetramethyl-1,3,2-dioxaborolan-2-yl)pyridin-2-amine

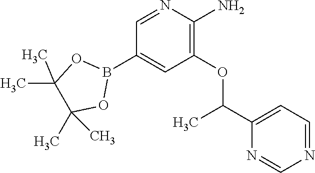 [SMILES: CC(Oc1cc(B2OC(C)(C)C(C)(C)O2)cnc1N)c1ccncn1]

(Rac)-5-Bromo-3-[1-(pyrimidin-4-yl)ethoxy]pyridin-2-amine (120 mg, 407 μmol) was dissolved in degasse 1,4-dioxane (3.6 mL). 4,4,4′,4′,5,5,5′,5′-Octamethyl-2,2′-bi-1,3,2-dioxaborolane (124 mg, 488 μmol), potassium acetate (119.7 mg, 1.2 mmol) and Pd(dppf)Cl₂ (29.7 mg, 41 μmol) were added and the mixture was stirred over night at 100° C. The reaction mixture was allowed to cool down, filtered and the filtrate was used in the next step without further work-up or purification.

### Intermediate 491

### 3-{[1-(2,4-dimethylpyrimidin-5-yl)ethyl]oxy}-5-(4,4,5,5-tetramethyl-1,3,2-dioxaborolan-2-yl)pyridin-2-amine (Enantiomer 2)

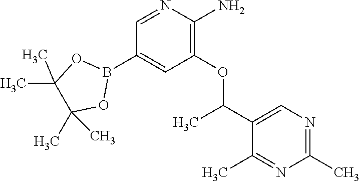

5-Bromo-3-{[1-(2,4-dimethylpyrimidin-5-yl)ethyl]oxy}pyridin-2-amine (enantiomer 2) (80.0 mg, 248 μmol) was dissolved In degassed 1,4-dioxane (2.2 mL). 4,4,4′,4′,5,5,5′,5′-Octamethyl-2,2′-bi-1,3,2-dioxaborolane (75.4 mg, 297 μmol), potassium acetate (73 mg, 0.7 mmol) and Pd(dppf)Cl₂ (18.1 mg, 25 μmol) were added and the mixture was stirred over night at 100° C. The reaction mixture was allowed to cool down, filtered and the filtrate was used in the next step without further work-up or purification.

LC-MS (Method 1): Rₜ=0.72 min; MS (ESIpos): m/z=371 [M+H]⁺

¹H-NMR (400 MHz, DMSO-d6) δ[ppm]: 1.066 (0.38), 1.156 (16.00), 1.164 (4.38), 1.231 (2.48), 1.236 (2.45), 1.293 (0.46), 1.550 (0.67), 1.565 (0.69), 1.586 (0.21), 2.467 (0.18), 2.523 (2.11), 3.566 (9.17), 6.287 (0.36), 7.036 (0.31), 7.039 (0.30), 7.777 (0.41), 7.781 (0.40), 8.733 (0.57).

### Intermediate 492

### 3-{[1-(2,4-dimethylpyrimidin-5-yl)ethyl]oxy}-5-(4,4,5,5-tetramethyl-1,3,2-dioxaborolan-2-yl)pyridin-2-amine (Enantiomer 1)

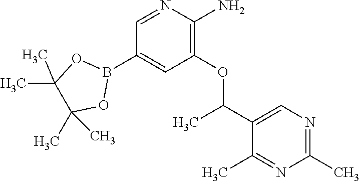

5-Bromo-3-{[1-(2,4-dimethylpyrimidin-5-yl)ethyl]oxy}pyridin-2-amine (enantiomer 1) (80.0 mg, 248 μmol) was dissolved in degassed 1,4-dioxane (2.2 mL). 4,4,4′,4′,5,5,5′,5′-Octamethyl-2,2′-bi-1,3,2-dioxaborolane (75.4 mg, 297 μmol), potassium acetate (73 mg, 0.7 mmol) and Pd(dppf)Cl₂ (18.1 mg, 25 μmol) were added and the mixture was stirred over night at 100° C. The reaction mixture was allowed to cool down, filtered and the filtrate was used in the next step without further work-up or purification.

LC-MS (Method 1): Rₜ=0.72 min; MS (ESIpos): m/z=371 [M+H]⁺

### Intermediate 493

### [6-amino-5-(6,7-dihydro-5H-cyclopenta[b]pyridin-7-yloxy)pyridin-3-yl]boronic acid (Enantiomer 1)

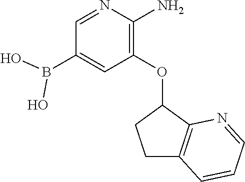 [SMILES: Nc1ncc(B(O)O)cc1OC1CCc2cccnc21]

5-Bromo-3-[(6,7-dihydro-5H-cyclopenta[b]pyridin-7-yl)oxy]pyridin-2-amine (enantiomer 1) (125 mg, 408 μmol), 4,4,4′,4′,5,5,5′,5′-octamethyl-2,2′-bi-1,3,2-dioxaborolane (124 mg, 490 μmol) and potassium acetate (120 mg, 1.22 mmol) were suspended in degassed 1,4-dioxane. Pd(dppf)Cl₂ (14.9 mg, 20.4 μmol) was added and the mixture was stirred for 2 h at 100° C. The reaction mixture was allowed to cool down, diluted with ethyl acetate and filtered. The filtrate was washed with water and brine, dried over sodium sulfate, filtered and concentrated to give 212 mg (76% yield, 40% purity) of the title compound which was used without further purification in the next step.

LC-MS (Method 1): Rₜ=0.53 min; MS (ESIpos): m/z=272 [M+H]⁺

### Intermediate 494

### 3-{1-[4-(methanesulfonyl)phenyl]ethoxy}-5-(4,4,5,5-tetramethyl-1,3,2-dioxaborolan-2-yl)pyridin-2-amine (Enantiomer 1 and enantiomer 2)

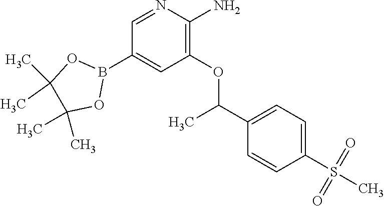

5-Bromo-3-{1-[4-(methanesulfonyl)phenyl]ethoxy}pyridin-2-amine (298 mg, 803 μmol), 4,4,4′,4′,5,5,5′,5′-octamethyl-2,2′-bi-1,3,2-dioxaborolane (224 mg, 883 μmol) and potassium acetate (236 mg, 2.41 mmol) were dissolved in degassed 1,4-dioxane (8 mL). Pd(dppf)Cl₂ (32.8 mg, 40.1 μmol) was added and the mixture was stirred over night at 100° C. The reaction mixture was allowed to cool down and filtered. The filtrate was diluted with ethyl acetate and washed with water. The aqueous phase was extracted with ethyl acetate. The combined organic layers were dried over sodium sulfate, filtered and concentrated to give 429 mg (128% yield) of the title compound.

LC-MS (Method 1): Rₜ=0.81 min; MS (ESIpos): m/z=419 [M+H]⁺

¹H-NMR (400 MHz, DMSO-d6) δ[ppm]: 1.065 (4.14), 1.156 (16.00), 1.164 (8.03), 1.219 (1.60), 1.292 (1.12), 1.901 (1.04), 2.518 (0.41), 3.202 (0.47), 3.206 (1.16), 3.566 (14.17).

### Intermediate 495

### di-tert-butyl [5-(4,4,5,5-tetramethyl-1,3,2-dioxaborolan-2-yl)-3-(trifluoromethyl)pyrazin-2-yl]-2-imidodicarbonate

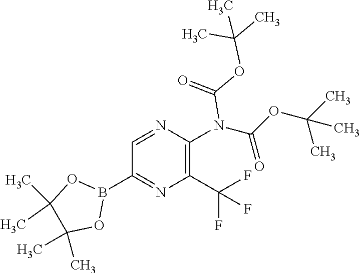 [SMILES: CC(C)(C)OC(=O)N(C(=O)OC(C)(C)C)c1ncc(B2OC(C)(C)C(C)(C)O2)nc1C(F)(F)F]

Di-tert-butyl [5-bromo-3-(trifluoromethyl)pyrazin-2-yl]-2-imidodicarbonate (200 mg, 452 μmol, prepared from commercially available 2-amino-5-bromo-3-trifluoromethylpyrazine according to WO2014096423 see the preparation of bis-tert-butoxycarbonyl derivative of 2-amino-5-bromo-3-methylpyrazine), 4,4,4′,4′,5,5,5′,5′-octamethyl-2,2′-bi-1,3,2-dioxaborolane (253 mg, 995 μmol) and potassium acetate (133 mg, 1.3 mmol) were dissolved in 1,4-dioxane (4.5 mL) under argon. Pd(dppf)Cl₂ (18.5 mg, 22.6 μmol) was added and the mixture was stirred for 2 h at 100° C. The reaction mixture was allowed to cool down, diluted with water (and a little bit of brine) and extracted with ethyl acetate. The combined organic layers were dried over a hydrophobic filter and concentrated. The residue was used in the next step without further purification.

### Intermediate 496

### 3-[(1R)-1-(2,6-difluorophenyl)ethoxy]-5-(4,4,5,5-tetramethyl-1,3,2-dioxaborolan-2-yl)pyridin-2-amine

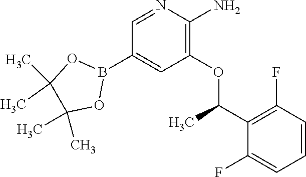 [SMILES: C[C@@H](Oc1cc(B2OC(C)(C)C(C)(C)O2)cnc1N)c1c(F)cccc1F]

5-Bromo-3-[(1R)-1-(2,6-difluorophenyl)ethoxy]pyridin-2-amine (196 mg, 595 μmol) and 4,4,4′,4′,5,5,5′,5′-octamethyl-2,2′-bi-1,3,2-dioxaborolane (181 mg, 715 μmol) were dissolved in degassed 1,4-dioxane und argon. Potassium acetate (175 mg, 1.79 mmol) and Pd(dppf)Cl₂ (21.8 mg, 29.8 μmol) were added and the mixture was stirred for 3.5 h at 100° C. The reaction mixture was allowed to cool down, diluted with ethyl acetate and filtered. The filtrate was washed with water and brine, dried over magnesium sulfate, filtered and concentrated to give 330 mg (49% purity, 72% yield) of the title compound which was used without further purification in the next step.

LC-MS (Method 1): Rₜ=1.26 min; MS (ESIpos): m/z=377 [M+H]⁺

¹H NMR (DMSO-d₆) δ: 7.76 (d, 1H), 7.36-7.45 (m, 1H), 7.05-7.13 (m, 2H), 7.04 (d, 1H), 6.02 (br s, 2H), 5.70-5.78 (m, 1H), 1.72 (m, 3H), 1.16 (s, 12H).

### Intermediate 497

### 3-[(1R)-1-phenylethoxy]-5-(4,4,5,5-tetramethyl-1,3,2-dioxaborolan-2-yl)pyrazin-2-amine

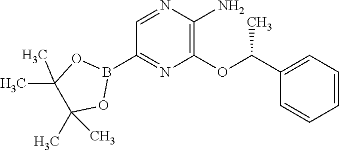 [SMILES: C[C@@H](Oc1nc(B2OC(C)(C)C(C)(C)O2)cnc1N)c1ccccc1]

5-Bromo-3-[(1R)-1-phenylethoxy]pyrazin-2-amine (37.0 mg, 126 μmol), 4,4,4′,4′,5,5,5′,5′-octamethyl-2,2′-bi-1,3,2-dioxaborolane (35.1 mg, 138 μmol) and potassium acetate (37.0 mg, 377 μmol) were combined in degassed 1,4-dioxane (1 mL). Pd(dppf)Cl₂ was added and the mixture was stirred for 4 h at 100° C. The reaction mixture was allowed to cool down, filtered and used without further work-up or purification in the next step.

LC-MS (Method 2): Rₜ=0.73 min; MS (ESIneg): m/z=339 [M−H]⁻

Suzuki

### Intermediate 498

### tert-butyl (3R)-2′-{6-amino-5-[(1R)-1-(3-fluoropyridin-2-yl)ethoxy]pyridin-3-yl}-5′,6′-dihydrospiro[pyrrolidine-3,4′-pyrrolo[1,2-b]pyrazole]-1-carboxylate

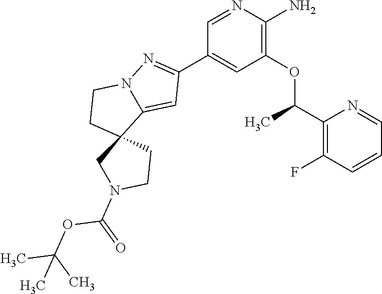 [SMILES: C[C@@H](Oc1cc(-c2cc3n(n2)CC[C@@]32CCN(C(=O)OC(C)(C)C)C2)cnc1N)c1ncccc1F]

Tert-butyl (3R)-2′-[(trifluoromethanesulfonyl)oxy]-5′,6′-dihydrospiro[pyrrolidine-3,4′-pyrrolo[1,2-b]pyrazole]-1-carboxylate (75.0 mg, 182 μmol), {6-amino-5-[(1R)-1-(3-fluoropyridin-2-yl)ethoxy]pyridin-3-yl}boronic acid (202 mg, 35% purity, 255 μmol) and potassium phosphate (1.1 mL, 0.50 M, 550 μmol) were dissolved in degassed 1,4-dioxane (3.0 mL) under argon. XPhos Pd G2 (7.17 mg, 9.11 μmol) was added and the mixture was stirred for 2 h at 100° C. The reaction mixture was allowed to cool down, diluted with ethyl acetate and filtered. The filtrate was washed with water and brine. The organic phase was dried over sodium sulfate, filtered and concentrated to give a residue. The crude product was purified by flash column chromatography (silica gel, dichloromethane/methanol (0-20%) gradient) to give 94.9 mg (60% purity, 63% yield) of the title compound.

LC-MS (Method 1): Rₜ=1.16 min; MS (ESIpos): m/z=496 [M+H]⁺

¹H-NMR (400 MHz, CHLOROFORM-d) δ [ppm]: 1.249 (16.00), 1.310 (0.41), 1.317 (0.41), 1.476 (1.41), 1.775 (0.35), 1.781 (0.80), 1.791 (0.37), 1.797 (0.79), 3.497 (0.38), 4.249 (0.21), 4.875 (0.39), 6.104 (0.20), 7.399 (0.17), 8.041 (0.18), 8.433 (0.20).

### Intermediate 499

### tert-butyl (3R)-2′-{6-amino-5-[(1R)-1-(3,5-difluoropyridin-4-yl)ethoxy]pyridin-3-yl}-5′,6′-dihydrospiro[pyrrolidine-3,4′-pyrrolo[1,2-b]pyrazole]-1-carboxylate

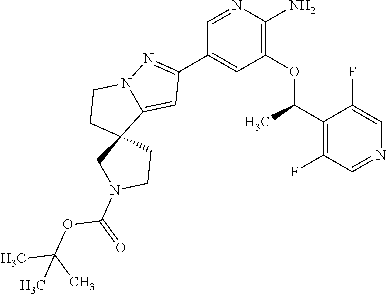 [SMILES: C[C@@H](Oc1cc(-c2cc3n(n2)CC[C@@]32CCN(C(=O)OC(C)(C)C)C2)cnc1N)c1c(F)cncc1F]

Tert-butyl (3R)-2′-[(trifluoromethanesulfonyl)oxy]-5′,6′-dihydrospiro[pyrrolidine-3,4′-pyrrolo[1,2-b]pyrazole]-1-carboxylate (61.2 mg, 149 μmol), {6-amino-5-[(1R)-1-(3,5-difluoropyridin-4-yl)ethoxy]pyridin-3-yl}boronic acid (143 mg, 43% purity, 208 μmol) and potassium phosphate (890 μL, 0.50 M, 450 μmol) were dissolved in degassed 1,4-dioxane (2.4 mL) under argon. XPhos Pd G2 (5.86 mg, 7.44 μmol) was added and the mixture was stirred for 2 h at 100° C. The reaction mixture was allowed to cool down, diluted with ethyl acetate and filtered. The filtrate was washed with water and brine. The organic phase was dried over sodium sulfate, filtered and concentrated to give a residue. The crude product was purified by flash column chromatography (silica gel, dichloromethane/methanol (0-20%) gradient) to give 108 mg (60% purity, 85% yield) of the title compound.

LC-MS (Method 1): Rₜ=1.18 min; MS (ESIpos): m/z=514 [M+H]⁺

### Intermediate 500

### tert-butyl (3R)-2′-{6-amino-5-[1-(1-methyl-1H-pyrazol-5-yl)ethoxy]pyridin-3-yl}-5′,6′-dihydro-1H-spiro[pyrrolidine-3,4′-pyrrolo[1,2-b]pyrazole]-1-carboxylate (Diastereomer 1 and Diastereomer 2)

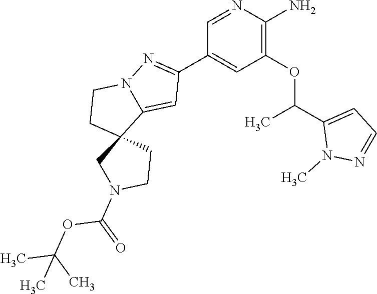 [SMILES: CC(Oc1cc(-c2cc3n(n2)CC[C@@]32CCN(C(=O)OC(C)(C)C)C2)cnc1N)c1ccnn1C]

Tert-butyl (3R)-2′-[(trifluoromethanesulfonyl)oxy]-5′,6′-dihydrospiro[pyrrolidine-3,4′-pyrrolo[1,2-b]pyrazole]-1-carboxylate (229 mg, 557 μmol), {6-amino-5-[1-(1-methyl-1H-pyrazol-5-yl)ethoxy]pyridin-3-yl}boronic acid (454 mg, 45% purity, 780 μmol) and potassium phosphate (3.3 mL, 0.50 M, 1.7 mmol) were dissolved in degassed 1,4-dioxane (9.2 mL). XPhos Pd G2 (21.9 mg, 27.8 μmol) was added and the reaction mixture was stirred for 2 h at 100° C. The reaction mixture was allowed to cool down, diluted with ethyl acetate and filtered. The filtrate was washed with water and brine. The organic phase was dried over sodium sulfate, filtered and concentrated. The residue was purified by column chromatography (silica gel, dichloromethane/methanol (0-15%) gradient) to give 152 mg (80% purity, 46% yield) of the title compound.

LC-MS (Method 1): Rₜ=1.06 min; MS (ESIpos): m/z=480 [M+H]⁺

¹H-NMR (400 MHz, DMSO-d6) δ[ppm]: 1.066 (1.11), 1.398 (12.26), 1.415 (3.26), 1.434 (11.74), 1.591 (1.85), 1.613 (6.70), 1.629 (6.51), 2.026 (1.27), 2.037 (1.44), 2.084 (0.60), 3.159 (6.69), 3.172 (6.77), 3.405 (5.59), 3.486 (0.62), 3.504 (1.14), 3.519 (0.79), 3.530 (0.68), 3.788 (3.93), 3.813 (16.00), 4.083 (0.64), 4.097 (1.74), 4.109 (1.82), 4.123 (0.84), 4.136 (1.11), 4.145 (1.36), 4.154 (2.01), 4.161 (2.00), 4.169 (1.35), 4.178 (1.09), 5.206 (0.43), 5.655 (0.86), 5.666 (0.64), 5.682 (0.43), 5.768 (4.77), 5.786 (1.67), 5.802 (1.39), 5.817 (0.51), 6.299 (1.40), 6.321 (1.53), 6.336 (0.85), 6.340 (0.83), 6.373 (3.32), 7.329 (2.62), 7.333 (3.52), 7.407 (2.59), 7.494 (0.50), 7.507 (0.52), 7.921 (3.20), 7.924 (3.21).

### Intermediate 501

### tert-butyl (3S)-2′-[6-amino-5-(trifluoromethyl)pyridin-3-yl]-5′,6′-dihydro-1H-spiro[pyrrolidine-3,4′-pyrrolo[1,2-b]pyrazole]-1-carboxylate

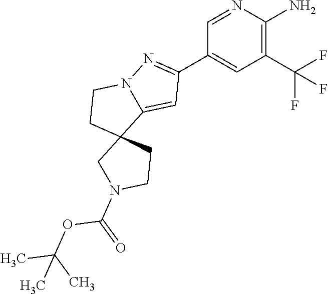 [SMILES: CC(C)(C)OC(=O)N1CC[C@]2(CCn3nc(-c4cnc(N)c(C(F)(F)F)c4)cc32)C1]

5-(4,4,5,5-Tetramethyl-1,3,2-dioxaborolan-2-yl)-3-(trifluoromethyl)pyridin-2-amine (1.50 g, 5.21 mmol) was dissolved in 1,4-dioxane. Tert-butyl (3S)-2′-[(trifluoromethanesulfonyl)oxy]-5′,6′-dihydrospiro[pyrrolidine-3,4′-pyrrolo[1,2-b]pyrazole]-1-carboxylate (2.36 g, 5.73 mmol) and potassium phosphate (31 mL, 0.50 M, 16 mmol) were added and the mixture was degassed with argon. XPhos Pd G2 (205 mg, 260 μmol) was added and the mixture was stirred for 2 h at 100° C. The reaction mixture was allowed to cool down, diluted with water and extracted 2 times with ethyl acetate. The combined organic layers were washed with brine, dried over sodium sulfate, filtered and concentrated to give a crude product. The residue was purified by column chromatography (silica gel, hexane/ethyl acetate (0-100%) gradient) to give 2.30 g (98% purity, 102% yield) of the title compound.

LC-MS (Method 1): Rₜ=1.20 min; MS (ESIpos): m/z=424 [M+H]⁺

¹H-NMR (400 MHz, DMSO-d6) δ[ppm]: 1.066 (8.94), 1.154 (0.49), 1.172 (0.96), 1.189 (0.46), 1.396 (16.00), 1.433 (12.64), 1.987 (1.68), 2.019 (0.73), 2.036 (1.54), 2.052 (1.25), 2.065 (0.83), 2.518 (1.64), 2.523 (1.43), 2.536 (1.28), 2.544 (1.64), 2.561 (0.93), 3.159 (7.02), 3.172 (5.86), 3.371 (0.45), 3.378 (0.42), 3.398 (1.58), 3.412 (5.51), 3.427 (0.95), 3.439 (0.81), 3.494 (0.60), 3.512 (1.23), 3.521 (0.59), 3.528 (0.71), 3.539 (0.74), 3.940 (1.49), 4.084 (0.51), 4.096 (1.49), 4.110 (1.55), 4.123 (0.56), 4.146 (0.41), 4.158 (1.15), 4.167 (1.28), 4.175 (2.02), 4.184 (1.99), 4.193 (1.26), 4.201 (1.06), 6.480 (1.73), 6.508 (2.23), 6.539 (4.73), 8.016 (2.36), 8.588 (2.64), 8.592 (2.56).

### Intermediate 502

### tert-butyl (3R)-2′-{6-amino-5-[(1R)-1-(pyridin-3-yl)ethoxy]pyridin-3-yl}-5′,6′-dihydrospiro[pyrrolidine-3,4′-pyrrolo[1,2-b]pyrazole]-1-carboxylate

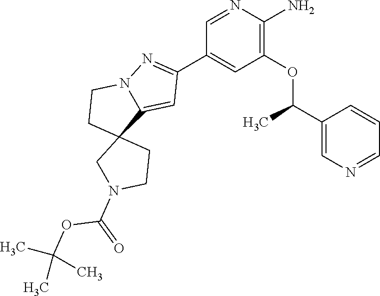 [SMILES: C[C@@H](Oc1cc(-c2cc3n(n2)CC[C@@]32CCN(C(=O)OC(C)(C)C)C2)cnc1N)c1cccnc1]

Tert-Butyl (3R)-2′-[(trifluoromethanesulfonyl)oxy]-5′,6′-dihydrospiro[pyrrolidine-3,4′-pyrrolo[1,2-b]pyrazole]-1-carboxylate (37.8 mg, 92.0 μmol), {6-amino-5-[(1R)-1-(pyridin-3-yl)ethoxy]pyridin-3-yl}boronic acid (220 mg, 13% purity, 110 μmol) and potassium phosphate (550 μl, 0.50 M, 280 μmol) were dissolved in degassed 1,4-dioxane (2.3 mL). XPhos Pd G2 (3.62 mg, 4.60 μmol) was added and the mixture was stirred for 3 h at 100° C. The reaction mixture was allowed to cool down, diluted with dichloromethane and filtered. The organic phase was washed with water and brine, dried and concentrated. The residue was purified by column chromatography (silica gel, dichloromethane/methanol (0-10%) gradient) to give 31.4 mg (72% yield) of the title compound.

LC-MS (Method 1): Rₜ=1.07 min; MS (ESIpos): m/z=477 [M+H]⁺

¹H-NMR (400 MHz, CHLOROFORM-d) 5 [ppm]: 1.174 (0.77), 1.183 (0.45), 1.388 (7.90), 1.427 (5.03), 1.596 (0.64), 1.614 (1.11), 1.629 (5.38), 1.645 (4.91), 1.936 (0.57), 1.950 (0.48), 2.513 (0.41), 2.530 (0.40), 3.360 (0.51), 3.419 (0.71), 3.444 (0.94), 3.452 (0.75), 3.678 (0.87), 4.136 (0.67), 4.152 (1.18), 4.171 (0.60), 4.697 (2.08), 5.233 (6.62), 5.447 (0.60), 5.463 (0.58), 5.999 (0.61), 6.024 (1.00), 7.194 (16.00), 7.205 (0.92), 7.216 (2.66), 7.220 (2.19), 7.236 (0.81), 7.634 (0.63), 7.653 (0.55), 7.922 (2.30), 7.926 (2.28), 8.463 (1.20), 8.467 (1.26), 8.475 (1.23), 8.479 (1.21), 8.601 (1.44), 8.606 (1.44).

### Intermediate 503

### tert-butyl (3R)-2′-{6-amino-5-[(1R)-1-(pyridin-3-yl)ethoxy]pyridin-3-yl}-5′,6′-dihydrospiro[pyrrolidine-3,4′-pyrrolo[1,2-b]pyrazole]-1-carboxylate

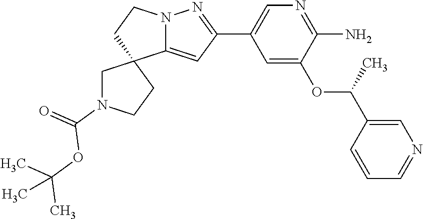 [SMILES: C[C@@H](Oc1cc(-c2cc3n(n2)CC[C@@]32CCN(C(=O)OC(C)(C)C)C2)cnc1N)c1cccnc1]

3-[(1R)-1-(Pyridin-3-yl)ethoxy]-5-(4,4,5,5-tetramethyl-1,3,2-dioxaborolan-2-yl)pyridin-2-amine (350 mg, 33% purity, 338 μmol) was dissolved in 1,4-dioxane (2 mL) under argon. Tert-Butyl (3R)-2′-[(trifluoromethanesulfonyl)oxy]-5′,6′-dihydrospiro[pyrrolidine-3,4′-pyrrolo[1,2-b]pyrazole]-1-carboxylate (127 mg, 308 μmol), potassium phosphate (1.8 ml, 0.50 M, 920 μmol) and XPhos Pd G2 (12.1 mg, 15.4 μmol) were added and the mixture was stirred for 1 h at 100° C. The reaction mixture was allowed to cool down and diluted with dichloromethane. The phases were separated, the organic phase was washed with brine, dried over a hydrophobic filter and concentrated. The residue was purified by preparative HPLC to give 73.0 mg (100% purity, 50% yield) of the title compound.

LC-MS (Method 1): Rₜ=1.04 min; MS (ESIpos): m/z=477 [M+H]⁺

¹H-NMR (400 MHz, DMSO-d₆): δ [ppm]=1.39-1.43 (m, 9H), 1.60 (d, 3H), 1.96-2.11 (m, 2H), 3.36-3.45 (m, 3H), 3.46-3.55 (m, 1H), 4.08-4.19 (m, 2H), 5.70 (q, 1H), 5.90 (s, 2H), 6.25-6.31 (m, 1H), 7.32 (s, 1H), 7.37 (dd, 1H), 7.87 (d, 1H), 7.89-7.94 (m, 1H), 8.47 (dd, 1H), 8.70 (d, 1H).

### Intermediate 504

### tert-butyl 2′-{6-amino-5-[(6,7-dihydro-5H-cyclopenta[b]pyridin-7-yl)oxy]pyridin-3-yl}-5′,6′-dihydrospiro[pyrrolidine-3,4′-pyrrolo[1,2-b]pyrazole]-1-carboxylate (4 Isomers)

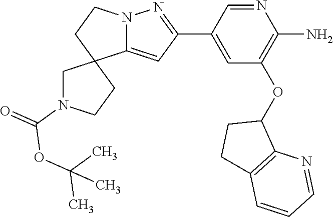 [SMILES: CC(C)(C)OC(=O)N1CCC2(CCn3nc(-c4cnc(N)c(OC5CCc6cccnc65)c4)cc32)C1]

Tert-Butyl 2′-[(trifluoromethanesulfonyl)oxy]-5′,6′-dihydrospiro[pyrrolidine-3,4′-pyrrolo[1,2-b]pyrazole]-1-carboxylate (249 mg, 605 μmol), 3-[(6,7-dihydro-5H-cyclopenta[b]pyridin-7-yl)oxy]-5-(4,4,5,5-tetramethyl-1,3,2-dioxaborolan-2-yl)pyridin-2-amine (346 mg, 71% purity, 695 μmol), dicyclohexyl[2′,4′,6′-tri(propan-2-yl)[1,1′-biphenyl]-2-yl]phosphane (28.8 mg, 60.5 μmol), XPhos Pd G2 (23.8 mg, 30.2 μmol) and potassium phosphate (3.6 ml, 0.50 M, 1.8 mmol) were combined in degassed 1,4-dioxane (10 mL) and stirred for 1 h at 100° C. The reaction mixture was allowed to cool down and diluted with dichloromethane and water. The phases were separated and the aqueous phase was extracted with dichloromethane/ethanol 9:1. The combined organic layers were dried and concentrated. The residue was purified by column chromatography (silica gel, ethyl acetate/ethanol (0-10%) gradient) to give 258 mg (87% yield) of the title compound.

LC-MS (Method 1): Rₜ=1.13 min; MS (ESIpos): m/z=489 [M+H]⁺

### Intermediate 505

### tert-butyl (3′R)-2-[6-amino-5-(difluoromethyl)pyridin-3-yl]-6,7-dihydrospiro[pyrazolo[5,1-c][1,4]oxazine-4,3′-pyrrolidine]-1′-carboxylate

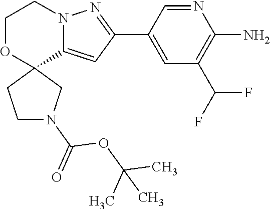 [SMILES: CC(C)(C)OC(=O)N1CC[C@]2(C1)OCCn1nc(-c3cnc(N)c(C(F)F)c3)cc12]

Tert-Butyl (3′R)-2-bromo-6,7-dihydrospiro[pyrazolo[5,1-c][1,4]oxazine-4,3′-pyrrolidine]-1′-carboxylate (527 mg, 1.47 mmol) and 3-(difluoromethyl)-5-(4,4,5,5-tetramethyl-1,3,2-dioxaborolan-2-yl)pyridin-2-amine (970 mg, 43% purity, 1.54 mmol, Intermediate 476) were dissolved in 1,4-dioxane. Potassium phosphate (937 mg, 4.41 mmol) was added and the mixture was degassed. XPhos Pd G2 (57.9 mg, 73.5 μmol) was added and the reaction mixture was stirred for 2 h at 100° C. The reaction mixture was allowed to cool down, diluted with ethyl acetate and filtered over celite. The organic phase was washed with water and brine, dried over a hydrophobic filter and concentrated. The residue was purified by column chromatography (silica gel, dichloromethane/methanol (0-4%) gradient) to give 490 mg (79% yield) of the title compound.

LC-MS (Method 1): Rₜ=1.10 min; MS (ESIpos): m/z=422 [M+H]⁺

¹H NMR (400 MHz, DMSO-d₆): δ [ppm]=1.41 (d, 9H), 2.14-2.31 (m, 2H), 3.35-3.59 (m, 3H), 3.68-3.77 (m, 1H), 3.94 (s, 1H), 4.11 (s, 4H), 6.38 (s, 2H), 6.70 (d, 1H), 6.91-7.22 (m, 1H), 7.95 (s, 1H), 8.43-8.51 (m, 1H).

### Intermediate 506

### tert-butyl (3′R)-2-(6-amino-5-cyanopyridin-3-yl)-6,7-dihydrospiro[pyrazolo[5,1-c][1,4]oxazine-4,3′-pyrrolidine]-1′-carboxylate

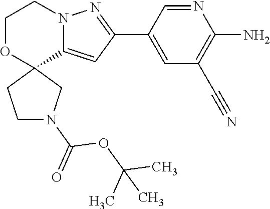 [SMILES: CC(C)(C)OC(=O)N1CC[C@]2(C1)OCCn1nc(-c3cnc(N)c(C#N)c3)cc12]

Tert-Butyl (3′R)-2-bromo-6,7-dihydrospiro[pyrazolo[5,1-c][1,4]oxazine-4,3′-pyrrolidine]-1′-carboxylate (300 mg, 837 μmol) and 2-amino-5-(4,4,5,5-tetramethyl-1,3,2-dioxaborolan-2-yl)pyridine-3-carbonitrile (216 mg, 879 μmol, CAS 1246372-66-6, Intermediate 2) were dissolved in 1,4-dioxane. Potassium phosphate (533 mg, 2.51 mmol) was added and the mixture was degassed. XPhos Pd G2 (32.9 mg, 41.9 μmol) was added and stirred for 2 h at 100° C. The reaction mixture was allowed to cool down, diluted with ethyl acetate and filtered over celite. The organic phase was washed with water and brine, dried over a hydrophobic filter and concentrated. The residue was purified by column chromatography (silica gel, dichloromethane/methanol (0-4%) gradient) to give 411 mg (80% purity, 99% yield) of the title compound.

LC-MS (Method 1): Rₜ=1.05 min; MS (ESIpos): m/z=397 [M+H]⁺

¹H-NMR (400 MHz, DMSO-d6) δ[ppm]: 1.066 (16.00), 1.156 (0.57), 1.401 (2.65), 1.426 (2.26), 2.085 (2.56), 2.518 (0.95), 2.523 (0.70), 3.938 (2.60), 4.115 (1.25), 7.045 (0.76), 8.592 (0.80), 8.597 (0.81).

### Intermediate 507

### tert-butyl (3′R)-2-[6-amino-5-(difluoromethoxy)pyridin-3-yl]-6,7-dihydrospiro[pyrazolo[5,1-c][1,4]oxazine-4,3′-pyrrolidine]-1′-carboxylate

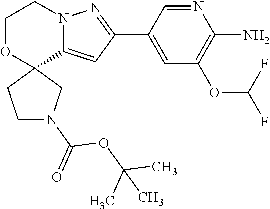 [SMILES: CC(C)(C)OC(=O)N1CC[C@]2(C1)OCCn1nc(-c3cnc(N)c(OC(F)F)c3)cc12]

Tert-Butyl (3′R)-2-bromo-6,7-dihydrospiro[pyrazolo[5,1-c][1,4]oxazine-4,3′-pyrrolidine]-1′-carboxylate (300 mg, 837 μmol) and 3-(difluoromethoxy)-5-(4,4,5,5-tetramethyl-1,3,2-dioxaborolan-2-yl)pyridin-2-amine (252 mg, 879 μmol, CAS 1188302-00-2) were dissolved in 1,4-dioxane. Potassium phosphate (533 mg, 2.51 mmol) was added and the mixture was degassed. XPhos Pd G2 (32.9 mg, 41.9 μmol) was added and stirred for 2 h at 100° C. The reaction mixture was allowed to cool down, diluted with ethyl acetate and filtered over celite. The organic phase was washed with water and brine, dried over a hydrophobic filter and concentrated. The residue was purified by column chromatography (silica gel, dichloromethane/methanol (0-4%) gradient) to give 292 mg (80% yield) of the title compound.

LC-MS (Method 1): Rₜ=1.11 min; MS (ESIpos): m/z=438 [M+H]⁺

¹H-NMR (400 MHz, DMSO-d6) δ[ppm]: 0.852 (0.50), 1.066 (3.19), 1.156 (1.91), 1.173 (0.48), 1.233 (0.92), 1.401 (16.00), 1.425 (13.52), 1.988 (0.93), 2.233 (0.41), 2.318 (1.01), 2.323 (1.68), 2.327 (2.19), 2.332 (1.52), 2.337 (0.78), 2.518 (6.10), 2.523 (4.43), 2.660 (0.54), 2.665 (1.24), 2.669 (1.73), 2.673 (1.17), 2.679 (0.50), 3.362 (0.58), 3.372 (0.57), 3.390 (0.49), 3.420 (0.71), 3.450 (0.75), 3.475 (0.68), 3.505 (0.91), 3.521 (0.68), 3.544 (1.17), 3.566 (0.58), 3.693 (0.63), 3.722 (1.00), 3.751 (0.44), 3.938 (0.55), 4.111 (7.31), 5.759 (11.94), 6.216 (4.46), 6.657 (1.90), 6.676 (2.24), 6.994 (1.95), 7.178 (3.89), 7.362 (1.69), 7.606 (2.39), 8.188 (2.88), 8.193 (2.82).

### Intermediate 508

### tert-butyl (3R)-2′-[6-amino-5-(trifluoromethoxy)pyridin-3-yl]-5′,6′-dihydrospiro[pyrrolidine-3,4′-pyrrolo[1,2-b]pyrazole]-1-carboxylate

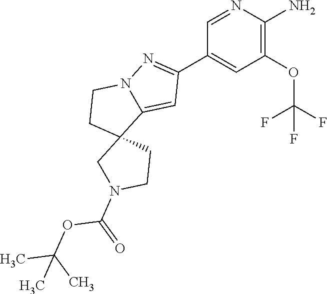 [SMILES: CC(C)(C)OC(=O)N1CC[C@@]2(CCn3nc(-c4cnc(N)c(OC(F)(F)F)c4)cc32)C1]

Tert-Butyl (3R)-2′-[(trifluoromethanesulfonyl)oxy]-5′,6′-dihydrospiro[pyrrolidine-3,4′-pyrrolo[1,2-b]pyrazole]-1-carboxylate (1.00 g, 2.43 mmol), [6-amino-5-(trifluoromethoxy)pyridin-3-yl]boronic acid (1.35 g, 40% purity, 2.43 mmol, intermediate 206) and potassium phosphate (15 mL, 0.50 M, 7.3 mmol) were dissolved in 1,4-dioxane. The mixture was degassed, XPhos Pd G2 (95.6 mg, 122 μmol) was added and the reaction mixture was stirred for 3 h at 100° C. The reaction mixture was allowed to cool down, diluted with water and extracted with ethyl acetate. The combined organic layers were dried over a hydrophobic filter and concentrated. The residue was purified by column chromatography (silica gel, hexane/ethyl acetate (0-100%) gradient) to give 932 mg (97% purity, 85% yield) of the title compound.

LC-MS (Method 1): Rₜ=1.21 min; MS (ESIpos): m/z=440 [M+H]⁺

¹H-NMR (400 MHz, DMSO-d6) δ[ppm]: 1.154 (5.17), 1.172 (10.41), 1.190 (4.91), 1.395 (16.00), 1.432 (12.57), 1.987 (15.97), 2.016 (0.72), 2.034 (1.29), 2.047 (1.19), 2.064 (0.84), 2.518 (3.08), 2.523 (2.64), 3.378 (0.56), 3.396 (1.84), 3.407 (6.01), 3.421 (0.94), 3.433 (0.73), 3.490 (0.65), 3.508 (1.18), 3.517 (0.61), 3.523 (0.72), 3.535 (0.70), 4.000 (1.19), 4.017 (3.69), 4.035 (3.69), 4.053 (1.19), 4.141 (0.40), 4.152 (1.12), 4.160 (1.25), 4.169 (2.01), 4.177 (2.01), 4.187 (1.21), 4.195 (1.05), 6.424 (1.71), 6.448 (2.18), 6.496 (4.96), 7.753 (2.30), 8.330 (5.16), 8.335 (4.75).

### Intermediate 509

### tert-butyl (3R)-2′-(6-amino-5-cyanopyridin-3-yl)-5′,6′-dihydrospiro[pyrrolidine-3,4′-pyrrolo[1,2-b]pyrazole]-1-carboxylate

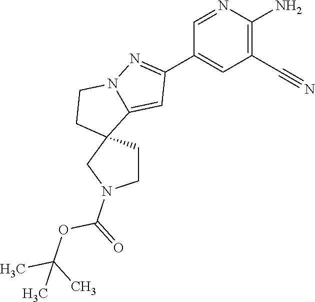 [SMILES: CC(C)(C)OC(=O)N1CC[C@@]2(CCn3nc(-c4cnc(N)c(C#N)c4)cc32)C1]

Tert-Butyl (3R)-2′-[(trifluoromethanesulfonyl)oxy]-5′,6′-dihydrospiro[pyrrolidine-3,4′-pyrrolo[1,2-b]pyrazole]-1-carboxylate (1.00 g, 2.43 mmol), 2-amino-5-(4,4,5,5-tetramethyl-1,3,2-dioxaborolan-2-yl)pyridine-3-carbonitrile (655 mg, 2.67 mmol, CAS 1246372-66-6) and potassium phosphate (15 mL, 0.50 M, 7.3 mmol) were dissolved in 1,4-dioxane (35 mL). The mixture was degassed. XPhos Pd G2 (95.6 mg, 122 μmol) was added and stirred for 3 h at 100° C. The reaction mixture was allowed to cool down, diluted with water and extracted with ethyl acetate. The combined organic layers were dried over a hydrophobic filter and concentrated. The residue was purified by column chromatography (silica gel, hexane/ethyl acetate (0-100%) gradient) to give 915 mg (100% purity, 99% yield) of the title compound.

LC-MS (Method 1): Rₜ=1.07 min; MS (ESIpos): m/z=381 [M+H]⁺

¹H NMR (DMSO-de, 400 MHz): δ (ppm) 8.61 (d, 1H), 8.15 (d, 1H), 6.99 (s, 2H), 6.46 and 6.48 (2s, 1H), 4.12-4.23 (m, 2H), 3.47-3.55 (m, 1H), 3.35-3.46 (m, 3H), 2.52-2.59 (m, 2H), 2.00-2.09 (m, 2H), 1.40 and 1.46 (2s, 9H).

### Intermediate 510

### tert-butyl (3R)-2′-[6-amino-5-(difluoromethoxy)pyridin-3-yl]-5′,6′-dihydrospiro[pyrrolidine-3,4′-pyrrolo[1,2-b]pyrazole]-1-carboxylate

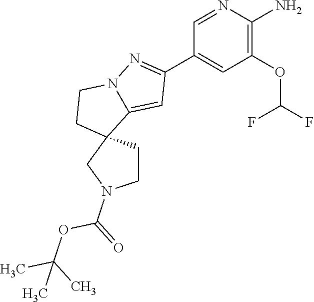 [SMILES: CC(C)(C)OC(=O)N1CC[C@@]2(CCn3nc(-c4cnc(N)c(OC(F)F)c4)cc32)C1]

Tert-Butyl (3R)-2′-[(trifluoromethanesulfonyl)oxy]-5′,6′-dihydrospiro[pyrrolidine-3,4′-pyrrolo[1,2-b]pyrazole]-1-carboxylate (1.00 g, 2.43 mmol), 3-(difluoromethoxy)-5-(4,4,5,5-tetramethyl-1,3,2-dioxaborolan-2-yl)pyridin-2-amine (765 mg, 2.67 mmol, CAS 1188302-00-2) and potassium phosphate (15 mL, 0.50 M, 7.3 mmol) were dissolved in 1,4-dioxane (35 mL). The mixture was degassed. XPhos Pd G2 (95.6 mg, 122 μmol) was added and stirred for 3 h at 100° C. The reaction mixture was allowed to cool down, diluted with water and extracted with ethyl acetate. The combined organic layers were dried over a hydrophobic filter and concentrated. The residue was purified by column chromatography (silica gel, hexane/ethyl acetate (0-100%) gradient) to give 996 mg (99% purity, 96% yield) of the title compound.

LC-MS (Method 1): Rₜ=1.12 min; MS (ESIpos): m/z=422 [M+H]⁺

¹H-NMR (400 MHz, DMSO-d6) δ[ppm]: 1.066 (0.70), 1.154 (1.84), 1.172 (3.62), 1.190 (1.69), 1.397 (16.00), 1.433 (12.83), 1.988 (6.38), 2.031 (1.09), 2.040 (1.13), 2.047 (1.15), 2.068 (0.78), 2.332 (0.49), 2.518 (3.16), 2.523 (2.57), 2.673 (0.48), 3.409 (6.16), 3.421 (1.00), 3.431 (0.72), 3.488 (0.63), 3.506 (1.16), 3.515 (0.61), 3.521 (0.76), 3.533 (0.68), 4.000 (0.48), 4.017 (1.45), 4.035 (1.44), 4.053 (0.46), 4.149 (1.12), 4.157 (1.28), 4.166 (2.05), 4.174 (2.10), 4.183 (1.26), 4.192 (1.12), 6.157 (5.25), 6.376 (1.77), 6.396 (2.25), 6.994 (2.43), 7.178 (4.89), 7.363 (2.10), 7.617 (2.81), 8.200 (5.35), 8.204 (5.02).

### Intermediate 511

### tert-butyl (3R)-2′-{6-amino-5-[(1R)-1-(2,6-dichloro-3-fluorophenyl)ethoxy]pyridin-3-yl}-5′,6′-dihydrospiro[pyrrolidine-3,4′-pyrrolo[1,2-b]pyrazole]-1-carboxylate

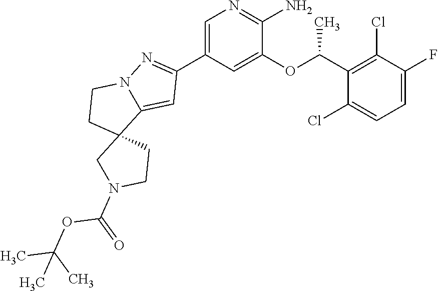 [SMILES: C[C@@H](Oc1cc(-c2cc3n(n2)CC[C@@]32CCN(C(=O)OC(C)(C)C)C2)cnc1N)c1c(Cl)ccc(F)c1Cl]

3-[(1R)-1-(2,6-Dichloro-3-fluorophenyl)ethoxy]-5-(4,4,5,5-tetramethyl-1,3,2-dioxaborolan-2-yl)pyridin-2-amine (560 mg, 1.31 mmol), tert-butyl (3R)-2′-[(trifluoromethanesulfonyl)oxy]-5′,6′-dihydrospiro[pyrrolidine-3,4′-pyrrolo[1,2-b]pyrazole]-1-carboxylate (593 mg, 1.44 mmol) and potassium phosphate (7.9 ml, 0.50 M, 3.9 mmol) were dissolved in 1,4-dioxane and the mixture was degassed. XPhos Pd G2 (51.5 mg, 65.6 μmol) was added and stirred for 2 h at 100° C. The reaction mixture was allowed to cool down, diluted with water and extracted with ethyl acetate. The combined organic layers were washed with brine, dried over sodium sulfate, filtered and concentrated. The residue was purified by column chromatography (silica gel, hexane/ethyl acetate (0-90%) and dichloromethane/methanol (0-10%) gradient) to give 445 mg (69% purity, 42% yield) of the title compound.

LC-MS (Method 1): Rₜ=1.38 min; MS (ESIpos): m/z=562 [M+H]⁺

¹H-NMR (400 MHz, DMSO-d6) δ[ppm]: 1.053 (0.66), 1.066 (9.51), 1.070 (0.72), 1.154 (2.72), 1.172 (5.70), 1.190 (2.97), 1.352 (0.53), 1.389 (13.38), 1.415 (8.44), 1.437 (7.44), 1.776 (4.21), 1.793 (4.21), 1.948 (0.49), 1.963 (0.54), 1.987 (11.53), 2.008 (0.96), 2.024 (0.95), 2.036 (0.84), 2.323 (0.46), 2.327 (0.65), 2.332 (0.50), 2.345 (0.62), 2.362 (1.00), 2.379 (0.62), 2.518 (2.43), 2.523 (1.59), 2.530 (0.79), 2.669 (0.53), 3.159 (15.51), 3.171 (16.00), 3.384 (2.74), 3.404 (0.99), 3.423 (0.99), 3.455 (0.52), 3.473 (0.65), 3.489 (0.42), 3.916 (0.68), 3.933 (1.30), 3.939 (1.03), 3.950 (0.68), 3.999 (0.84), 4.017 (2.44), 4.034 (2.40), 4.052 (0.80), 4.089 (1.47), 4.098 (2.75), 4.112 (3.12), 4.124 (2.02), 4.142 (0.73), 4.148 (0.72), 5.206 (3.17), 5.787 (1.71), 6.035 (0.41), 6.051 (0.44), 6.133 (0.89), 6.157 (1.04), 7.066 (0.61), 7.081 (0.69), 7.417 (0.78), 7.438 (1.59), 7.460 (1.12), 7.530 (0.71), 7.542 (0.77), 7.552 (0.61), 7.565 (0.52), 7.882 (0.88), 9.578 (0.63).

### Intermediate 512

### tert-butyl (3R)-2′-{6-amino-5-[(1R)-1-(pyridin-4-yl)ethoxy]pyridin-3-yl}-5′,6′-dihydrospiro[pyrrolidine-3,4′-pyrrolo[1,2-b]pyrazole]-1-carboxylate

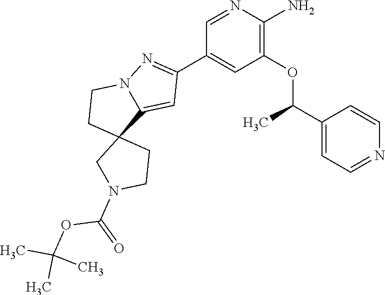 [SMILES: C[C@@H](Oc1cc(-c2cc3n(n2)CC[C@@]32CCN(C(=O)OC(C)(C)C)C2)cnc1N)c1ccncc1]

Tert-Butyl (3R)-2′-[(trifluoromethanesulfonyl)oxy]-5′,6′-dihydrospiro[pyrrolidine-3,4′-pyrrolo[1,2-b]pyrazole]-1-carboxylate (188 mg, 456 μmol), {6-amino-5-[(1R)-1-(pyridin-4-yl)ethoxy]pyridin-3-yl}boronic acid (500 mg, 26% purity, 502 μmol, Intermediate 480) and potassium phosphate (2.2 ml, 1.4 mmol) were dissolved in degassed 1,4-dioxane (2.3 mL). XPhos Pd G2 (17.9 mg, 22.8 μmol) was added and the mixture was stirred for 2 h at 100° C. {6-Amino-5-[(1R)-1-(pyridin-4-yl)ethoxy]pyridin-3-yl}boronic acid (152 mg, 26% purity, 139 μmol), potassium phosphate (0.7 ml, 0.4 mmol) and XPhos Pd G2 (10 mg, 12.7 μmol) were added again and stirred for 1 h at 100° C. The reaction mixture was allowed to cool down, diluted with ethyl acetate and filtered. The filtrate was washed with water and brine. The organic phase was dried and concentrated. The residue was purified by column chromatography (silica gel, dichloromethane/methanol (0-10%) gradient) to give 54.0 mg (25% yield) of the title compound.

LC-MS (Method 1): Rₜ=1.27 min; MS (ESIpos): m/z=477 [M+H]⁺

### Intermediate 513

### tert-butyl (3R)-2′-{6-amino-5-[(1R)-1-phenylethoxy]pyridin-3-yl}-5′,6′-dihydrospiro[pyrrolidine-3,4′-pyrrolo[1,2-b]pyrazole]-1-carboxylate

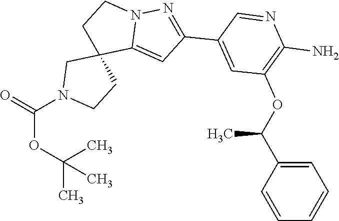 [SMILES: C[C@@H](Oc1cc(-c2cc3n(n2)CC[C@@]32CCN(C(=O)OC(C)(C)C)C2)cnc1N)c1ccccc1]

3-[(1R)-1-Phenylethoxy]-5-(4,4,5,5-tetramethyl-1,3,2-dioxaborolan-2-yl)pyridin-2-amine (476 mg, 1.40 mmol), tert-butyl (3R)-2′-[(trifluoromethanesulfonyl)oxy]-5′,6′-dihydrospiro[pyrrolidine-3,4′-pyrrolo[1,2-b]pyrazole]-1-carboxylate (634 mg, 1.54 mmol) and potassium phosphate (8.4 ml, 0.50 M, 4.2 mmol) were dissolved in degassed 1,4-dioxane (15 mL). XPhos Pd G2 (55.1 mg, 70.0 μmol) was added and the mixture was stirred for 1 h at 100° C. The reaction mixture was allowed to reach rt, diluted with water and extracted with ethyl acetate. The combined organic layers were washed with brine, dried over sodium sulfate, filtered and concentrated. The residue was purified by column chromatography (silica gel, hexane/ethyl acetate (20-100%) and ethyl acetate/methanol (5-10%) gradient) to give 377 mg (57% yield) of the title compound.

LC-MS (Method 1): Rₜ=1.27 min; MS (ESIpos): m/z=476 [M+H]⁺

¹H-NMR (400 MHz, DMSO-d6) δ[ppm]: 1.154 (4.32), 1.172 (9.21), 1.190 (4.57), 1.389 (16.00), 1.432 (12.28), 1.563 (7.30), 1.579 (7.26), 1.988 (15.51), 2.005 (1.13), 2.017 (1.26), 2.108 (0.45), 2.518 (3.24), 2.523 (2.41), 3.364 (1.51), 3.378 (5.02), 3.405 (1.43), 3.468 (0.50), 3.487 (0.91), 3.498 (0.64), 3.513 (0.56), 3.999 (1.14), 4.017 (3.42), 4.035 (3.29), 4.053 (1.05), 4.102 (1.05), 4.110 (1.10), 4.120 (1.92), 4.126 (1.80), 4.137 (1.16), 4.144 (1.04), 5.552 (0.41), 5.568 (1.36), 5.584 (1.38), 5.599 (0.42), 5.833 (4.23), 6.192 (0.52), 6.224 (1.59), 6.245 (2.01), 7.231 (2.78), 7.245 (2.16), 7.263 (1.52), 7.319 (2.56), 7.338 (4.69), 7.352 (0.88), 7.357 (2.32), 7.453 (4.00), 7.471 (3.17), 7.475 (2.28), 7.838 (3.72), 7.842 (3.63).

### Intermediate 514

### tert-butyl (3′R)-2-{5-[bis(tert-butoxycarbonyl)amino]-6-(trifluoromethyl)pyrazin-2-yl}-6,7-dihydrospiro[pyrazolo[5,1-c][1,4]oxazine-4,3′-pyrrolidine]-1′-carboxylate

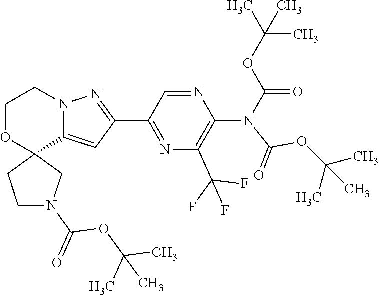 [SMILES: CC(C)(C)OC(=O)N1CC[C@]2(C1)OCCn1nc(-c3cnc(N(C(=O)OC(C)(C)C)C(=O)OC(C)(C)C)c(C(F)(F)F)n3)cc12]

{5-[Bis(tert-butoxycarbonyl)amino]-6-(trifluoromethyl)pyrazin-2-yl}boronic acid (184 mg, 452 μmol, Intermediate 495), tert-butyl (3′R)-2-bromo-6,7-dihydrospiro[pyrazolo[5,1-c][1,4]oxazine-4,3′-pyrrolidine]-1′-carboxylate (154 mg, 430 μmol) and potassium phosphate (2.6 mL, 0.50 M, 1.3 mmol) were suspended in 1,4-dioxane (4.3 mL) under argon. XPhos Pd G2 (16.9 mg, 21.5 μmol) was added and the mixture was stirred for 1.5 h at 100° C. The reaction mixture was allowed to cool down, diluted with water and extracted with ethyl acetate. The combined organic layers were dried over a hydrophobic filter and concentrated. The residue was purified by column chromatography (silica gel NH2, hexane/ethyl acetate (0-100%) and ethyl acetate/ethanol (0-65%) gradient) to give 168 mg (80% purity, 49% yield) of the title compound.

LC-MS (Method 1): Rₜ=1.58 min; MS (ESIpos): m/z=542 [M-Boc+2H]⁺

### Intermediate 515

### tert-butyl (3R)-2′-{5-[bis(tert-butoxycarbonyl)amino]-6-(trifluoromethyl)pyrazin-2-yl}-5′,6′-dihydrospiro[pyrrolidine-3,4′-pyrrolo[1,2-b]pyrazole]-1-carboxylate

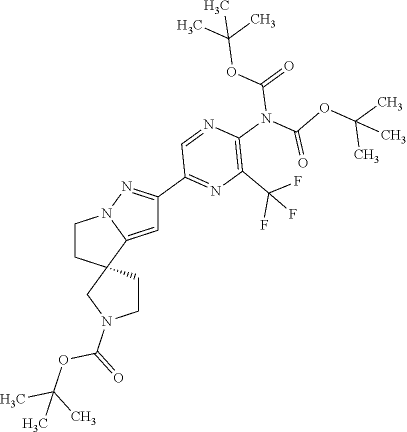 [SMILES: CC(C)(C)OC(=O)N1CC[C@@]2(CCn3nc(-c4cnc(N(C(=O)OC(C)(C)C)C(=O)OC(C)(C)C)c(C(F)(F)F)n4)cc32)C1]

{5-[Bis(tert-butoxycarbonyl)amino]-6-(trifluoromethyl)pyrazin-2-yl}boronic acid (92.0 mg, 226 μmol, Intermediate 495), tert-butyl (3R)-2′-[(trifluoromethanesulfonyl)oxy]-5′,6′-dihydrospiro[pyrrolidine-3,4′-pyrrolo[1,2-b]pyrazole]-1-carboxylate (88.5 mg, 215 μmol) and potassium phosphate (5.2 mL, 0.50 M, 2.58 mmol) were dissolved in degassed 1,4-dioxane (2.2 mL) under argon. XPhos Pd G2 (8.47 mg, 10.8 μmol) was added and the mixture was stirred for 1.5 h at 100° C. The reaction mixture was allowed to cool down, diluted with water and extracted with ethyl acetate. The combined organic layers were dried over a hydrophobic filter and concentrated. The residue was purified by column chromatography (silica gel NH2, hexane/ethyl acetate (0-100%) and ethyl acetate/ethanol (0-65%) gradient) to give 54 mg (38% yield) of the title compound.

LC-MS (Method 1): Rₜ=1.59 min; MS (ESIpos): m/z=626 [M+H]⁺

### Intermediate 516

### tert-butyl (3R)-2′-{6-amino-5-[(1R)-1-(2,6-difluorophenyl)ethoxy]pyridin-3-yl}-5′,6′-dihydrospiro[pyrrolidine-3,4′-pyrrolo[1,2-b]pyrazole]-1-carboxylate

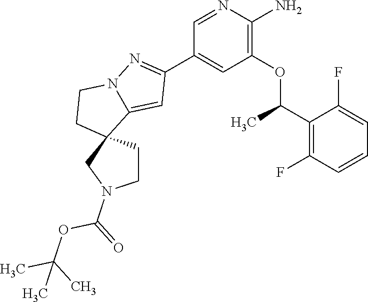 [SMILES: C[C@@H](Oc1cc(-c2cc3n(n2)CC[C@@]32CCN(C(=O)OC(C)(C)C)C2)cnc1N)c1c(F)cccc1F]

3-[(1R)-1-(2,6-difluorophenyl)ethoxy]-5-(4,4,5,5-tetramethyl-1,3,2-dioxaborolan-2-yl)pyridin-2-amine (327 mg, 49% purity, 426 μmol) was dissolved in degassed 1,4-dioxane (7 mL). Tert-Butyl (3R)-2′-[(trifluoromethanesulfonyl)oxy]-5′,6′-dihydrospiro[pyrrolidine-3,4′-pyrrolo[1,2-b]pyrazole]-1-carboxylate (175 mg, 426 μmol), potassium phosphate (2.6 mL, 0.50 M, 1.3 mmol) and XPhos Pd G2 (16.8 mg, 21.3 μmol) were added and the mixture was stirred for 2 h at 100° C. The reaction mixture was allowed to cool down, diluted with dichloromethane and washed with water and brine. The organic phase was dried over a hydrophobic filter and concentrated. The residue was purified by preparative HPLC to give 111 mg (91% purity, 46% yield) of the title compound.

LC-MS (Method 1): Rₜ=1.26 min; MS (ESIpos): m/z=512 [M+H]⁺

¹H NMR (DMSO-de, 400 MHz): δ (ppm) 7.89 (s, 1H), 7.36-7.44 (m, 1H), 7.30 (d, 1H), 7.07-7.13 (m, 2H), 6.21 and 6.22 (2s, 1H), 5.81 (q, 1H), 5.67 (s, 2H), 4.09-4.20 (m, 2H), 3.46-3.54 (m, 1H), 3.37-3.45 (m, 3H), 2.12 (s, 2H), 1.75 (d, 3H), 1.40 and 1.44 (2s, 9H).

BOC-Abspaltung

### Intermediate 517

### 5-[(3R)-5′,6′-dihydrospiro[pyrrolidine-3,4′-pyrrolo[1,2-b]pyrazol]-2′-yl]-3-[(1R)-1-(3-fluoropyridin-2-yl)ethoxy]pyridin-2-amine-hydrochloride

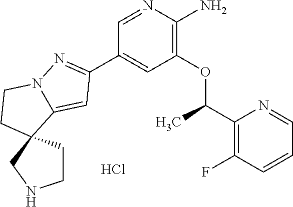 [SMILES: C[C@@H](Oc1cc(-c2cc3n(n2)CC[C@@]32CCNC2)cnc1N)c1ncccc1F ; Cl]

Tert-butyl (3R)-2′-{6-amino-5-[(1R)-1-(3-fluoropyridin-2-yl)ethoxy]pyridin-3-yl}-5′,6′-dihydrospiro[pyrrolidine-3,4′-pyrrolo[1,2-b]pyrazole]-1-carboxylate (92.0 mg, 60% purity, 112 μmol) was dissolved in 1,4-dioxane (1.8 mL). Hydrochloric acid in 1,4-dioxane (280 μL, 4.0 M, 1.1 mmol) was added and the mixture was stirred for 1 h at 40° C. The reaction mixture was concentrated to dryness to give 142 mg (35% purity, 103% yield) of the title compound.

LC-MS (Method 1): Rₜ=0.81 min; MS (ESIpos): m/z=396 [M+H]⁺

### Intermediate 518

### 3-[(1R)-1-(3,5-difluoropyridin-4-yl)ethoxy]-5-[(3R)-5′,6′-dihydrospiro[pyrrolidine-3,4′-pyrrolo[1,2-b]pyrazol]-2′-yl]pyridin-2-amine-hydrochloride

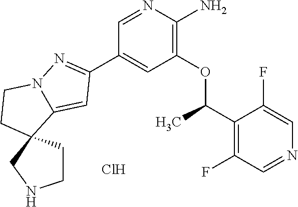 [SMILES: C[C@@H](Oc1cc(-c2cc3n(n2)CC[C@@]32CCNC2)cnc1N)c1c(F)cncc1F ; Cl]

Tert-butyl (3R)-2′-{6-amino-5-[(1R)-1-(3,5-difluoropyridin-4-yl)ethoxy]pyridin-3-yl}-5′,6′-dihydrospiro[pyrrolidine-3,4′-pyrrolo[1,2-b]pyrazole]-1-carboxylate (108 mg, 60% purity, 126 μmol) was dissolved in 1,4-dioxane (2.1 mL). Hydrochloric acid in 1,4-dioxane (320 μL, 4.0 M, 1.3 mmol) was added and the mixture was stirred for 1 h at 40° C. The reaction mixture was concentrated to dryness to give 139 mg (40% purity, 98% yield) of the title compound.

LC-MS (Method 1): Rₜ=0.84 min; MS (ESIpos): m/z=414 [M+H]⁺

### Intermediate 519

### 5-[(3R)-5′,6′-dihydrospiro[pyrrolidine-3,4′-pyrrolo[1,2-b]pyrazol]-2′-yl]-3-[1-(1-methyl-1H-pyrazol-5-yl)ethoxy]pyridin-2-amine-hydrochloride (Diastereomer 1 and Diastereomer 2)

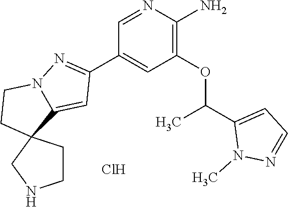 [SMILES: CC(Oc1cc(-c2cc3n(n2)CC[C@@]32CCNC2)cnc1N)c1ccnn1C ; Cl]

Tert-butyl (3R)-2′-{6-amino-5-[1-(1-methyl-1H-pyrazol-5-yl)ethoxy]pyridin-3-yl}-5′,6′-dihydro-1H-spiro[pyrrolidine-3,4′-pyrrolo[1,2-b]pyrazole]-1-carboxylate (150 mg, 80% purity, 250 μmol) was dissolved in 1,4-dioxane (4.1 mL). Hydrochloric acid in 1,4-dioxane (630 μL, 4.0 M, 2.5 mmol) was added and the reaction mixture was stirred for 1 h at 40° C. The reaction mixture was concentrated to give 120 mg (80% purity, 92% yield) of the title compound which was used without further purification in the next step.

LC-MS (Method 1): Rₜ=0.75 min; MS (ESIpos): m/z=381 [M+H]⁺

### Intermediate 520

### trifluoroacetic acid-5-(5′,6′-dihydrospiro[azetidine-3,4′-pyrrolo[1,2-b]pyrazol]-2′-yl)-3-[(1R)-1-phenylethoxy]pyridin-2-amine

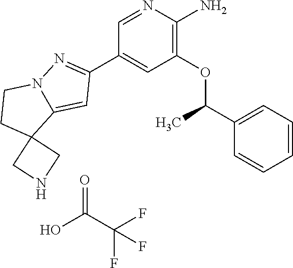

Tert-butyl 2′-{6-amino-5-[(1R)-1-phenylethoxy]pyridin-3-yl}-5′,6′-dihydrospiro[azetidine-3,4′-pyrrolo[1,2-b]pyrazole]-1-carboxylate (2.39 g, 5.18 mmol) was dissolved in dichloromethane (25 mL). Trifluoroacetic acid (25 mL, 320 mmol) was added and the mixture was stirred for 1 h at rt. The reaction mixture was concentrated to give 5.1 g (207% yield) of the title compound.

LC-MS (Method 2): Rₜ=0.59 min; MS (ESIpos): m/z=362 [M+H]⁺

¹H NMR (400 MHz, DMSO-d₆, 22° C.) δ ppm 1.61 (d, 3H), 2.91 (t, 2H), 4.09 (t, 2H), 4.18 (br s, 4H), 5.72 (q, 1H), 6.63 (s, 1H), 7.23-7.29 (m, 1H), 7.35 (t, 2H), 7.42 (s, 1H), 7.46-7.50 (m, 2H), 7.84 (d, 1H), 9.04 (br s, 2H).

### Intermediate 521

### 5-[(3R)-5′,6′-dihydrospiro[pyrrolidine-3,4′-pyrrolo[1,2-b]pyrazol]-2′-yl]-3-[(1R)-1-(pyridin-3-yl)ethoxy]pyridin-2-amine hydrogen chloride (1/1)

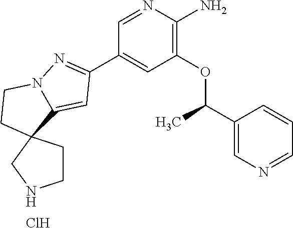 [SMILES: C[C@@H](Oc1cc(-c2cc3n(n2)CC[C@@]32CCNC2)cnc1N)c1cccnc1 ; Cl]

Tert-Butyl (3R)-2′-{6-amino-5-[(1R)-1-(pyridin-3-yl)ethoxy]pyridin-3-yl}-5′,6′-dihydrospiro[pyrrolidine-3,4′-pyrrolo[1,2-b]pyrazole]-1-carboxylate (31.0 mg, 65.0 μmol) was dissolved in 1,4-dioxane (0.8 mL). Hydrochloric acid (110 μL, 4M in 1,4-dioxane) was added and the mixture was stirred for 1 h at room temperature. The reaction mixture was concentrated to give 36.0 mg (134% yield) of the title compound.

LC-MS (Method 1): Rₜ=0.76 min; MS (ESIpos): m/z=377 [M+H]⁺

¹H-NMR (400 MHz, DMSO-d6) δ[ppm]: 1.107 (0.50), 1.232 (0.54), 1.593 (16.00), 1.664 (2.10), 1.680 (2.28), 1.987 (0.54), 2.153 (0.47), 2.174 (0.50), 2.327 (1.06), 2.332 (0.77), 2.518 (5.23), 2.523 (3.40), 2.669 (1.18), 2.673 (0.89), 3.384 (0.70), 3.429 (0.42), 3.990 (0.45), 4.185 (0.60), 4.200 (0.76), 4.214 (0.77), 4.230 (0.56), 6.658 (2.30), 7.797 (0.91), 7.841 (1.11), 7.844 (0.96), 8.677 (0.43), 8.690 (0.46), 8.960 (0.53).

### Intermediate 522

### 3-(6,7-dihydro-5H-cyclopenta[b]pyridin-7-yloxy)-5-(5′,6′-dihydrospiro[pyrrolidine-3,4′-pyrrolo[1,2-b]pyrazol]-2′-yl)pyridin-2-amine dihydrochloride (4 Isomers)

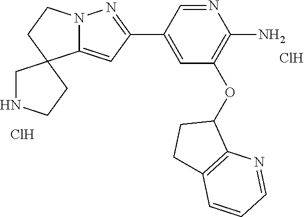 [SMILES: Cl ; Nc1ncc(-c2cc3n(n2)CCC32CCNC2)cc1OC1CCc2cccnc21 ; Cl]

Tert-Butyl 2′-{6-amino-5-[(6,7-dihydro-5H-cyclopenta[b]pyridin-7-yl)oxy]pyridin-3-yl}-5′,6′-dihydrospiro[pyrrolidine-3,4′-pyrrolo[1,2-b]pyrazole]-1-carboxylate (308 mg, 630 μmol) was dissolved in 1,4-dioxane (16 mL) and methanol (1.6 mL). Hydrochloric acid in 1,4-dioxane (630 μL, 4.0 M, 2.5 mmol) was added and the mixture was stirred over the weekend at rt. The reaction mixture was concentrated, diluted with toluene and evaporated again to give 446 mg (153% yield) of the title compound.

LC-MS (Method 1): Rₜ=0.82 min; MS (ESIpos): m/z=389 [M+H]⁺

### Intermediate 523

### 3-(difluoromethyl)-5-[(3′R)-6,7-dihydrospiro[pyrazolo[5,1-c][1,4]oxazine-4,3′-pyrrolidin]-2-yl]pyridin-2-amine

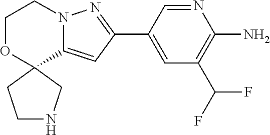 [SMILES: Nc1ncc(-c2cc3n(n2)CCO[C@@]32CCNC2)cc1C(F)F]

Tert-butyl (3′R)-2-[6-amino-5-(difluoromethyl)pyridin-3-yl]-6,7-dihydrospiro[pyrazolo[5,1-c][1,4]oxazine-4,3′-pyrrolidine]-1′-carboxylate (490 mg, 1.16 mmol, Intermediate 505) was dissolved in 1,4-dioxane. Hydrochloric acid in 1,4-dioxane (2.9 mL, 4.0 M, 12 mmol) was added and the mixture was stirred for 1 h at room temperature. Hydrochloric acid in 1,4-dioxane (2.9 mL, 4.0 M, 12 mmol) was added again and the reaction mixture was stirred for 2 h at room temperature. The reaction mixture was concentrated. The residue was diluted with ethyl acetate and extracted with 2 M sodium hydroxide solution. The aqueous phase was extracted with ethyl acetate. The combined organic layers were dried over a hydrophobic filter and concentrated to give 275 mg (95% purity, 70% yield) of the title compound.

LC-MS (Method 1): Rₜ=0.72 min; MS (ESIpos): m/z=322 [M+H]⁺

### Intermediate 524

### 2-amino-5-[(3′R)-6,7-dihydrospiro[pyrazolo[5,1-c][1,4]oxazine-4,3′-pyrrolidin]-2-yl]pyridine-3-carbonitrile

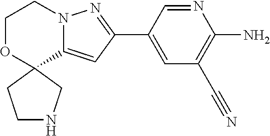 [SMILES: N#Cc1cc(-c2cc3n(n2)CCO[C@@]32CCNC2)cnc1N]

Tert-Butyl (3′R)-2-(6-amino-5-cyanopyridin-3-yl)-6,7-dihydrospiro[pyrazolo[5,1-c][1,4]oxazine-4,3′-pyrrolidine]-1′-carboxylate (411 mg, 80% purity, 829 μmol, Intermediate 506) was dissolved in 1,4-dioxane. Hydrochloric acid in 1,4-dioxane (2.1 mL, 4.0 M, 8.3 mmol) was added and the mixture was stirred for 1 h at room temperature. The reaction mixture was concentrated. The residue was dissolved in ethyl acetate and extracted with 2 M sodium hydroxide solution. The aqueous phase was extracted with ethyl acetate. The combined organic layers were dried over a hydrophobic filter and concentrated to give 215 mg (100% purity, 87% yield) of the title compound.

LC-MS (Method 1): Rₜ=0.67 min; MS (ESIpos): m/z=297 [M+H]⁺

### Intermediate 525

### 3-(difluoromethoxy)-5-[(3′R)-6,7-dihydrospiro[pyrazolo[5,1-c][1,4]oxazine-4,3′-pyrrolidin]-2-yl]pyridin-2-amine

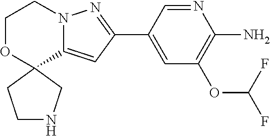 [SMILES: Nc1ncc(-c2cc3n(n2)CCO[C@@]32CCNC2)cc1OC(F)F]

Tert-Butyl (3′R)-2-[6-amino-5-(difluoromethoxy)pyridin-3-yl]-6,7-dihydrospiro[pyrazolo[5,1-c][1,4]oxazine-4,3′-pyrrolidine]-1′-carboxylate (292 mg, 668 μmol, Intermediate 507) was dissolved in 1,4-dioxane. Hydrochloric acid in 1,4-dioxane (1.7 mL, 4.0 M, 6.7 mmol) was added and the mixture was stirred for 1 h at room temperature. Hydrochloric acid in 1,4-dioxane (1.7 mL, 4.0 M, 6.7 mmol) was added again and the resulting mixture was stirred for 2 h at room temperature. The reaction mixture was concentrated. The residue was dissolved in ethyl acetate and extracted with 2 M sodium hydroxide solution. The aqueous phase was extracted with ethyl acetate. The combined organic layers were dried over a hydrophobic filter and concentrated to give 208 mg (92% purity, 85% yield) of the title compound.

LC-MS (Method 1): Rₜ=0.73 min; MS (ESIpos): m/z=338 [M+H]⁺

### Intermediate 526

### 5-(5′,6′-dihydrospiro[azetidine-3,4′-pyrrolo[1,2-b]pyrazol]-2′-yl)-3-(trifluoromethyl)pyridin-2-amine

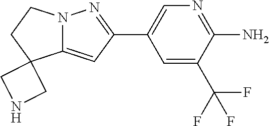 [SMILES: Nc1ncc(-c2cc3n(n2)CCC32CNC2)cc1C(F)(F)F]

5-(5′,6′-Dihydrospiro[azetidine-3,4′-pyrrolo[1,2-b]pyrazol]-2′-yl)-3-(trifluoromethyl)pyridin-2-amine hydrogen chloride (1/1) (830 mg, 2.40 mmol) was suspended in ethyl acetate and extracted with 2 M sodium hydroxide solution. The aqueous phase was extracted with ethyl acetate. The combined organic layers were dried and concentrated to give 583 mg (79% yield) of the title compound.

LC-MS (Method 1): Rₜ=0.81 min; MS (ESIpos): m/z=310 [M+H]⁺

¹H NMR (DMSO-d₆, 400 MHz): δ (ppm) 8.61-8.63 (m, 1H), 8.03 (d, 1H), 6.67 (s, 1H), 6.53 (s, 1H), 4.05-4.11 (m, 2H), 3.73 (d, 2H), 3.58 (d, 2H), 2.78-2.83 (m, 2H).

### Intermediate 527

### 5-[(3R)-5′,6′-dihydrospiro[pyrrolidine-3,4′-pyrrolo[1,2-b]pyrazol]-2′-yl]-3-(trifluoromethoxy)pyridin-2-amine hydrogen chloride (1/1)

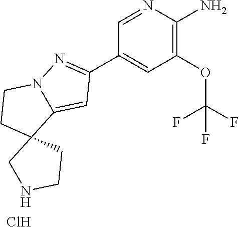 [SMILES: Nc1ncc(-c2cc3n(n2)CC[C@@]32CCNC2)cc1OC(F)(F)F ; Cl]

Tert-Butyl (3R)-2′-[6-amino-5-(trifluoromethoxy)pyridin-3-yl]-5′,6′-dihydrospiro[pyrrolidine-3,4′-pyrrolo[1,2-b]pyrazole]-1-carboxylate (929 mg, 2.11 mmol, Intermediate 508) was dissolved in 1,4-dioxane (9 mL). Hydrochloric acid in 1,4-dioxane (5.3 mL, 4.0 M, 21 mmol) was added dropwise and stirred for 1.5 h at room temperature. The reaction mixture was concentrated to give 888 mg (98% purity, 110% yield) of the title compound.

LC-MS (Method 1): Rₜ=0.85 min; MS (ESIpos): m/z=340 [M+H]⁺

¹H NMR (400 MHz, DMSO-d₆, 22° C.) δ ppm=2.09-2.22 (m, 2H), 2.53-2.62 (m, 1H), 2.68-2.77 (m, 1H), 3.22-3.49 (m, 4H), 4.17-4.25 (m, 2H), 6.68 (s, 1H), 8.03 (s, 1H), 8.33 (d, 1H), 9.67-9.79 (m, 2H).

### Intermediate 528

### 2-amino-5-[(3R)-5′,6′-dihydrospiro[pyrrolidine-3,4′-pyrrolo[1,2-b]pyrazol]-2′-yl]pyridine-3-carbonitrile hydrogen chloride (1/1)

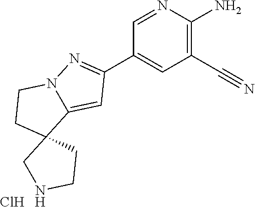 [SMILES: N#Cc1cc(-c2cc3n(n2)CC[C@@]32CCNC2)cnc1N ; Cl]

Tert-Butyl (3R)-2′-(6-amino-5-cyanopyridin-3-yl)-5′,6′-dihydrospiro[pyrrolidine-3,4′-pyrrolo[1,2-b]pyrazole]-1-carboxylate (915 mg, 2.41 mmol, Intermediate 509) was dissolved in 1,4-dioxane (10 mL). Hydrochloric acid in 1,4-dioxane (6.0 ml, 4.0 M, 24 mmol) was added dropwise and the mixture was stirred over night at room temperature. The reaction mixture was concentrated to give 852 mg (112% yield) of the title compound.

LC-MS (Method 1): Rₜ=0.69 min; MS (ESIpos): m/z=281 [M+H]⁺

¹H NMR (400 MHz, DMSO-d₆) δ ppm=2.10-2.22 (m, 2H), 2.53-2.61 (m, 1H), 2.68-2.76 (m, 1H), 3.21-3.48 (m, 4H), 4.14-4.25 (m, 2H), 6.63 (s, 1H), 8.23 (d, 1H), 8.59 (d, 1H), 9.67-9.87 (m, 2H).

### Intermediate 529

### 3-(difluoromethoxy)-5-[(3R)-5′,6′-dihydrospiro[pyrrolidine-3,4′-pyrrolo[1,2-b]pyrazol]-2′-yl]pyridin-2-amine hydrogen chloride (1/1)

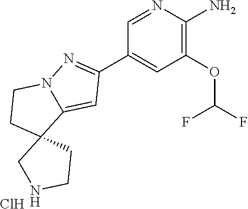 [SMILES: Nc1ncc(-c2cc3n(n2)CC[C@@]32CCNC2)cc1OC(F)F ; Cl]

Tert-Butyl (3R)-2′-[6-amino-5-(difluoromethoxy)pyridin-3-yl]-5′,6′-dihydrospiro[pyrrolidine-3,4′-pyrrolo[1,2-b]pyrazole]-1-carboxylate (996 mg, 2.36 mmol, Intermediate 510) was dissolved in 1,4-dioxane (10 mL). Hydrochloric acid in 1,4-dioxane (5.9 ml, 4.0 M, 24 mmol) was added dropwise and the mixture was stirred over night at room temperature. The reaction mixture was concentrated to give 996 mg (100% purity, 118% yield) of the title compound.

LC-MS (Method 1): Rₜ=0.76 min; MS (ESIpos): m/z=322 [M+H]⁺

¹H NMR (400 MHz, DMSO-d₆) δ ppm=2.10-2.23 (m, 2H), 2.54-2.62 (m, 1H), 2.70-2.78 (m, 1H), 3.22-3.47 (m, 4H), 4.17-4.28 (m, 2H), 6.72 (s, 1H), 7.42 (t, 1H), 8.03-8.05 (m, 1H), 8.19 (d, 1H), 9.85 (br s, 2H).

### Intermediate 530

### 5-[(3S)-5′,6′-dihydrospiro[pyrrolidine-3,4′-pyrrolo[1,2-b]pyrazol]-2′-yl]-3-(trifluoromethyl)pyridin-2-amine hydrogen chloride (1/1)

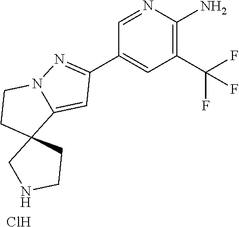 [SMILES: Nc1ncc(-c2cc3n(n2)CC[C@]32CCNC2)cc1C(F)(F)F ; Cl]

Tert-Butyl (3S)-2′-[6-amino-5-(trifluoromethyl)pyridin-3-yl]-5′,6′-dihydrospiro[pyrrolidine-3,4′-pyrrolo[1,2-b]pyrazole]-1-carboxylate (1.19 g, 2.81 mmol) was dissolved in 1,4-dioxane (14 mL). Hydrochloric acid in 1,4-dioxane (14 ml, 4.0 M, 56 mmol) was added and the mixture was stirred over night at room temperature. The reaction mixture was concentrated to give 1.17 g (116% yield) of the title compound.

LC-MS (Method 1): Rₜ=0.84 min; MS (ESIpos): m/z=324 [M+H]⁺

¹H-NMR (400 MHz, DMSO-d6) δ[ppm]: 1.064 (4.59), 2.083 (1.74), 2.131 (0.53), 2.144 (1.48), 2.156 (1.54), 2.163 (3.38), 2.170 (2.26), 2.183 (1.94), 2.188 (1.87), 2.518 (5.37), 2.523 (4.15), 2.562 (1.04), 2.566 (0.88), 2.579 (1.60), 2.594 (1.80), 2.613 (1.04), 2.679 (1.46), 2.698 (1.85), 2.714 (1.46), 2.727 (0.87), 2.731 (1.03), 2.746 (0.68), 3.242 (0.57), 3.257 (1.00), 3.271 (1.17), 3.286 (1.52), 3.304 (0.94), 3.319 (0.48), 3.330 (0.68), 3.347 (1.06), 3.367 (0.90), 3.384 (2.71), 3.392 (1.00), 3.402 (1.22), 3.408 (1.31), 3.420 (1.72), 3.437 (1.52), 3.447 (1.52), 3.467 (0.87), 3.486 (0.52), 4.173 (0.54), 4.185 (1.91), 4.192 (2.00), 4.200 (2.78), 4.204 (2.70), 4.207 (2.52), 4.212 (2.54), 4.219 (2.01), 4.227 (1.80), 4.239 (0.48), 5.079 (1.26), 6.668 (16.00), 8.109 (3.93), 8.113 (3.88), 8.137 (0.68), 8.585 (4.13), 8.588 (3.93), 9.579 (0.93).

### Intermediate 531

### 3-[(1R)-1-(2,6-dichloro-3-fluorophenyl)ethoxy]-5-[(3R)-5′,6′-dihydrospiro[pyrrolidine-3,4′-pyrrolo[1,2-b]pyrazol]-2′-yl]pyridin-2-amine hydrogen chloride (1/1)

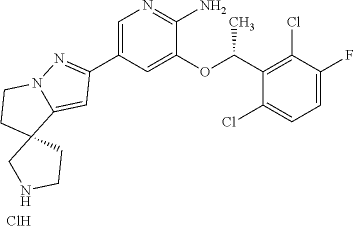 [SMILES: C[C@@H](Oc1cc(-c2cc3n(n2)CC[C@@]32CCNC2)cnc1N)c1c(Cl)ccc(F)c1Cl ; Cl]

Tert-Butyl (3R)-2′-{6-amino-5-[(1R)-1-(2,6-dichloro-3-fluorophenyl)ethoxy]pyridin-3-yl}-5′,6′-dihydrospiro[pyrrolidine-3,4′-pyrrolo[1,2-b]pyrazole]-1-carboxylate (445 mg, 69% purity, 546 μmol) was dissolved in 1,4-dioxane (2.7 mL). Hydrochloric acid in 1,4-dioxane ((2.7 ml, 4.0 M, 11 mmol) was added and the mixture was stirred over night at room temperature. The reaction mixture was concentrated to give 299 mg (110% yield) of the title compound.

¹H-NMR (400 MHz, DMSO-d6) δ[ppm]: 1.064 (3.89), 1.230 (0.59), 1.247 (0.59), 1.592 (2.74), 1.839 (5.89), 1.855 (5.78), 1.906 (0.52), 2.068 (2.00), 2.085 (3.81), 2.104 (2.30), 2.118 (0.63), 2.138 (1.63), 2.158 (1.19), 2.167 (1.04), 2.322 (0.81), 2.326 (1.15), 2.332 (0.78), 2.384 (0.56), 2.389 (0.52), 2.402 (1.11), 2.417 (1.00), 2.435 (0.67), 2.518 (5.22), 2.522 (4.07), 2.541 (1.44), 2.547 (1.19), 2.558 (1.07), 2.563 (1.22), 2.573 (0.78), 2.581 (0.63), 2.664 (1.26), 2.669 (1.37), 2.673 (1.04), 2.682 (1.11), 2.699 (0.78), 2.715 (0.67), 3.205 (0.85), 3.220 (1.22), 3.233 (1.59), 3.250 (1.44), 3.266 (0.70), 3.299 (1.33), 3.307 (1.67), 3.316 (1.70), 3.328 (1.59), 3.345 (1.78), 3.363 (1.48), 3.384 (4.33), 3.395 (1.85), 3.406 (1.22), 3.549 (2.15), 3.666 (16.00), 3.751 (2.41), 3.929 (1.33), 3.936 (1.37), 3.947 (1.96), 3.956 (1.63), 3.963 (1.33), 3.970 (1.26), 4.152 (1.33), 4.168 (2.30), 4.187 (1.44), 5.435 (9.33), 6.228 (1.37), 6.245 (1.37), 6.526 (7.07), 7.353 (2.59), 7.356 (2.67), 7.461 (1.26), 7.483 (2.44), 7.504 (1.96), 7.553 (0.44), 7.572 (1.48), 7.584 (1.56), 7.594 (1.15), 7.607 (0.96), 7.827 (2.81), 7.831 (2.81), 8.181 (0.78), 9.502 (0.52), 9.690 (0.56).

### Intermediate 532

### 5-[(3R)-5′,6′-dihydrospiro[pyrrolidine-3,4′-pyrrolo[1,2-b]pyrazol]-2′-yl]-3-[(1R)-1-(pyridin-4-yl)ethoxy]pyridin-2-amine hydrogen chloride (1/1)

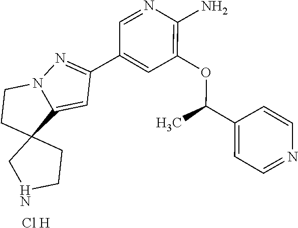

Tert-Butyl (3R)-2′-{6-amino-5-[(1R)-1-(pyridin-4-yl)ethoxy]pyridin-3-yl}-5′,6′-dihydrospiro[pyrrolidine-3,4′-pyrrolo[1,2-b]pyrazole]-1-carboxylate (54.0 mg, 113 μmol, Intermediate 512) was dissolved in 1,4-dioxane (1.5 mL). Hydrochloric acid in 1,4-dioxane (170 μL, 680 μmol, 4M) was added slowly and the mixture was stirred for 1 h at room temperature. The reaction mixture was concentrated to give 80.0 mg (58% purity, 99% yield) of the title compound.

LC-MS (Method 1): Rₜ=0.77 min; MS (ESIpos): m/z=377 [M+H]⁺

¹H-NMR (400 MHz, DMSO-d6) δ[ppm]: 0.850 (0.41), 1.063 (1.61), 1.105 (0.76), 1.154 (0.74), 1.230 (2.97), 1.247 (1.51), 1.388 (0.69), 1.428 (0.56), 1.591 (16.00), 1.618 (0.42), 1.648 (8.27), 1.665 (8.22), 1.906 (0.87), 2.083 (0.65), 2.109 (0.59), 2.121 (0.94), 2.142 (2.00), 2.160 (2.39), 2.178 (1.26), 2.562 (1.73), 2.577 (1.60), 2.595 (0.84), 2.685 (0.76), 2.705 (1.33), 2.720 (1.19), 2.736 (0.84), 2.753 (0.49), 3.190 (0.45), 3.208 (0.92), 3.221 (1.01), 3.237 (1.16), 3.252 (0.65), 3.326 (0.89), 3.337 (0.89), 3.384 (2.61), 3.414 (1.87), 3.423 (1.69), 3.456 (0.72), 3.467 (0.61), 3.487 (0.54), 3.587 (0.56), 3.675 (0.45), 3.699 (0.45), 3.712 (0.40), 4.017 (0.45), 4.088 (0.43), 4.102 (0.46), 4.112 (0.45), 4.180 (2.09), 4.193 (2.91), 4.206 (2.07), 4.318 (0.69), 4.327 (0.54), 4.652 (0.40), 5.758 (3.85), 6.219 (1.21), 6.235 (1.23), 6.668 (7.60), 7.742 (3.35), 7.887 (4.27), 7.890 (4.20), 8.111 (2.61), 8.124 (2.72), 8.137 (1.05), 8.361 (1.53), 8.896 (3.49), 8.910 (3.30), 9.789 (0.75), 9.872 (0.74).

### Intermediate 533

### 5-[(3R)-5′,6′-dihydrospiro[pyrrolidine-3,4′-pyrrolo[1,2-b]pyrazol]-2′-yl]-3-[(1R)-1-phenylethoxy]pyridin-2-amine

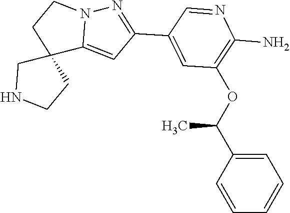 [SMILES: C[C@@H](Oc1cc(-c2cc3n(n2)CC[C@@]32CCNC2)cnc1N)c1ccccc1]

Tert-Butyl (3R)-2′-{6-amino-5-[(1R)-1-phenylethoxy]pyridin-3-yl}-5′,6′-dihydrospiro[pyrrolidine-3,4′-pyrrolo[1,2-b]pyrazole]-1-carboxylate (377 mg, 793 μmol) was dissolved in 1,4-dioxane (4 mL). Hydrochloric acid in 1,4-dioxane (4.0 ml, 4.0 M, 16 mmol) was added and the mixture was stirred for 1 h at room temperature. The reaction mixture was concentrated. The residue was purified by preparative HPLC to give 140 mg (47% yield) of the title compound.

LC-MS (Method 1): Rₜ=0.96 min; MS (ESIpos): m/z=376 [M+H]⁺

¹H NMR (400 MHz, DMSO-d₆) δ ppm=1.57 (d, 3H), 1.82-1.97 (m, 2H), 2.36-2.44 (m, 1H), 2.75-2.80 (m, 1H), 2.83-2.88 (m, 1H), 2.92-3.02 (m, 2H), 4.01-4.12 (m, 2H), 5.58 (q, 1H), 5.81 (s, 2H), 6.23 (s, 1H), 7.22-7.27 (m, 2H), 7.31-7.36 (m, 2H), 7.44-7.49 (m, 2H), 7.83 (d, 1H).

### Intermediate 534

### 5-[(3′R)-6,7-dihydrospiro[pyrazolo[5,1-c][1,4]oxazine-4,3′-pyrrolidin]-2-yl]-3-(trifluoromethyl)pyrazin-2-amine

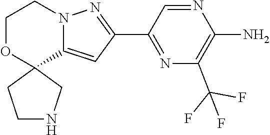 [SMILES: Nc1ncc(-c2cc3n(n2)CCO[C@@]32CCNC2)nc1C(F)(F)F]

Tert-Butyl (3′R)-2-{5-[bis(tert-butoxycarbonyl)amino]-6-(trifluoromethyl)pyrazin-2-yl}-6,7-dihydrospiro[pyrazolo[5,1-c][1,4]oxazine-4,3′-pyrrolidine]-1′-carboxylate (168 mg, 262 μmol, Intermediate 514) was dissolved in 1,4-dioxane (2.6 mL). Hydrochloric acid in 1,4-dioxane (1.3 mL, 4.0 M, 5.2 mmol) was added and the mixture was stirred for 16 h at room temperature. The reaction mixture was concentrated to give 100 mg (90% purity, 101% yield) of the title compound.

LC-MS (Method 1): Rₜ=0.79 min; MS (ESIpos): m/z=342 [M+H]⁺

¹H-NMR (400 MHz, DMSO-d6) δ[ppm]: 1.134 (16.00), 1.300 (0.46), 1.317 (0.26), 1.387 (0.36), 1.500 (0.39), 2.392 (0.26), 2.396 (0.38), 2.401 (0.35), 2.426 (0.20), 2.435 (0.25), 2.450 (0.18), 2.461 (0.36), 2.485 (0.28), 2.515 (0.32), 2.587 (1.54), 2.592 (0.99), 2.734 (0.25), 2.738 (0.33), 2.743 (0.24), 3.350 (0.19), 3.369 (0.28), 3.381 (0.39), 3.399 (0.35), 3.453 (0.24), 3.492 (0.33), 3.505 (0.29), 3.524 (0.51), 3.543 (0.39), 3.555 (0.36), 3.567 (0.32), 3.634 (1.00), 3.718 (0.35), 3.733 (0.41), 3.749 (0.33), 3.986 (2.06), 4.191 (0.99), 4.225 (0.98), 4.236 (1.14), 4.260 (1.16), 4.268 (0.88), 4.273 (0.93), 6.411 (0.59), 6.416 (0.62), 6.871 (3.55), 7.079 (0.18), 7.548 (0.53), 7.553 (0.51), 8.206 (0.19), 8.824 (1.75), 9.491 (0.22), 9.678 (0.19).

### Intermediate 535

### 5-[(3R)-5′,6′-dihydrospiro[pyrrolidine-3,4′-pyrrolo[1,2-b]pyrazol]-2′-yl]-3-(trifluoromethyl)pyrazin-2-amine

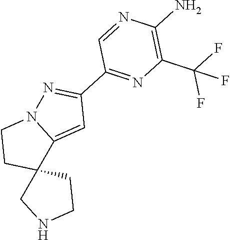 [SMILES: Nc1ncc(-c2cc3n(n2)CC[C@@]32CCNC2)nc1C(F)(F)F]

Tert-Butyl (3R)-2′-{5-[bis(tert-butoxycarbonyl)amino]-6-(trifluoromethyl)pyrazin-2-yl}-5′,6′-dihydrospiro[pyrrolidine-3,4′-pyrrolo[1,2-b]pyrazole]-1-carboxylate (399 mg, 639 μmol, Intermediate 515) was dissolved in 1,4-dioxane (6.3 mL). Hydrochloric acid in 1,4-dioxane (3.2 mL, 4.0 M, 13 mmol) was added and the mixture was stirred for 16 h at room temperature. The reaction mixture was concentrated to give 243 mg (90% purity, 106% yield) of the title compound.

LC-MS (Method 1): Rₜ=0.81 min; MS (ESIpos): m/z=326 [M+H]⁺

¹H-NMR (400 MHz, DMSO-d6) δ[ppm]: 1.064 (16.00), 1.231 (0.55), 2.171 (0.69), 2.190 (0.77), 2.518 (1.21), 2.523 (0.69), 2.726 (0.41), 3.353 (0.56), 3.367 (0.82), 3.372 (0.76), 3.383 (0.90), 3.564 (6.04), 4.210 (0.42), 4.215 (0.41), 4.227 (0.75), 4.235 (0.54), 4.245 (0.42), 4.250 (0.42), 4.528 (0.93), 6.634 (3.33), 8.736 (1.43), 8.738 (1.44).

### Intermediate 536

### 3-[(1R)-1-(2,6-difluorophenyl)ethoxy]-5-[(3R)-5′,6′-dihydrospiro[pyrrolidine-3,4′-pyrrolo[1,2-b]pyrazol]-2′-yl]pyridin-2-amine-hydrogen chloride (1/1)

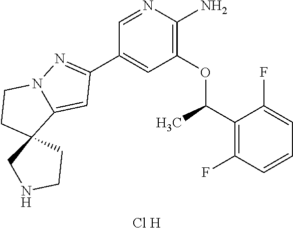 [SMILES: C[C@@H](Oc1cc(-c2cc3n(n2)CC[C@@]32CCNC2)cnc1N)c1c(F)cccc1F ; "Cl H"]

Tert-Butyl (3R)-2′-{6-amino-5-[(1R)-1-(2,6-difluorophenyl)ethoxy]pyridin-3-yl}-5′,6′-dihydrospiro[pyrrolidine-3,4′-pyrrolo[1,2-b]pyrazole]-1-carboxylate (110 mg, 215 μmol) was dissolved in 1,4-dioxane (0.9 mL). Hydrochloric acid in 1,4-dioxane (540 μL, 4.0 M, 2.2 mmol) was added and the mixture was stirred for 20 h at 40° C. The reaction mixture allowed to cool down and concentrated to give 120 mg (76% purity, 94% yield) of the title compound.

LC-MS (Method 1): Rₜ=0.96 min; MS (ESIpos): m/z=412 [M+H]⁺

¹H NMR (DMSO-de, 400 MHz): δ (ppm) 9.56-9.70 (m, 2H), 7.89 (br s, 2H), 7.83 (d, 1H), 7.60 (d, 1H), 7.40-7.49 (m, 1H), 7.09-7.18 (m, 2H), 6.55 (s, 1H), 6.04 (q, 1H), 4.15-4.26 (m, 2H), 3.21-3.29 (m, 1H), 2.65-2.74 (m, 1H), 2.53-2.61 (m, 1H), 2.09-2.23 (m, 2H), 1.81 (d, 3H).

Neutralisation

### Intermediate 537

### 5-[(3′R)-6,7-dihydrospiro[pyrazolo[5,1-c][1,4]oxazine-4,3′-pyrrolidin]-2-yl]-3-(trifluoromethyl)pyridin-2-amine

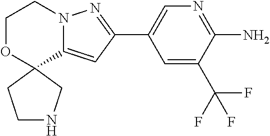 [SMILES: Nc1ncc(-c2cc3n(n2)CCO[C@@]32CCNC2)cc1C(F)(F)F]

(3′R)-2-[6-Amino-5-(trifluoromethyl)pyridin-3-yl]-6,7-dihydrospiro[pyrazolo[5,1-c][1,4]oxazine-4,3′-pyrrolidin[1]ium] chloride (100 mg, 266 μmol, intermediate 319) was suspended in ethyl acetate and washed with 2 M sodium hydroxide solution. The organic phase was washed with water and brine, dried over sodium sulfate, filtered and concentrated to give 71.7 mg (100% purity, 79% yield) of the title compound.

LC-MS (Method 1): Rₜ=0.81 min; MS (ESIpos): m/z=340 [M+H]⁺

Other

### Intermediate 538

### 2-[(2-methylbut-3-yn-2-yl)oxy]oxane (Enantiomer 1 and Enantiomer 2)

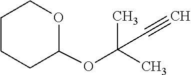

2-Methylbut-3-yn-2-ol (2.00 g, 23.8 mmol) was dissolved in dichloromethane (24 mL) and cooled to 0° C. 3,4-Dihydro-2H-pyran (2.8 mL, 30 mmol) and toluene-4-sulfonic acid monohydrate (12.3 mg, 71.3 μmol) were added and the mixture was stirred over night at room temperature. The reaction mixture was diluted with saturated sodium bicarbonate solution and the phases were separated. The aqueous phase was extracted with dichloromethane. The combined organic layers were dried and concentrated to give 3.96 g (99% yield) of the title compound.

¹H NMR (400 MHz, DMSO-d₆) δ ppm=1.35-1.54 (m, 10H), 1.55-1.65 (m, 1H), 1.66-1.77 (m, 1H), 3.38-3.45 (m, 1H), 3.45 (s, 1H), 3.80 (ddd, 1H), 5.01 (dd, 1H).

### Intermediate 539

### 1-(3-{3-methyl-3-[(-oxan-2-yl)oxy]but-1-yn-1-yl}phenyl)ethan-1-ol (Mixture of 4 Isomers)

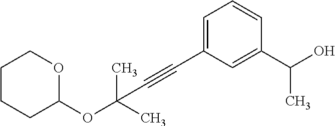 [SMILES: CC(O)c1cccc(C#CC(C)(C)OC2CCCCO2)c1]

Triethylamine (500 μL, 3.6 mmol) was dissolved in degassed 1,4-dioxane. 2-[(2-Methylbut-3-yn-2-yl)oxy]oxane (182 mg, 1.08 mmol), 1-(3-iodophenyl)ethan-1-ol (295 mg, 1.19 mmol), dichloridopalladium-triphenylphosphane (1/2) (75.9 mg, 108 μmol) and copper (1) iodide (61.8 mg, 324 μmol) were added and the mixture was stirred over night at room temperature. The reaction mixture was concentrated. The crude product was purified by column chromatography (silica gel, dichloromethane/methanol (0-5%) gradient) to give 267 mg (85% yield) of the title compound.

LC-MS (Method 1): Rₜ=1.28 min; MS (ESIneg): m/z=287 [M−H]⁻

### Intermediate 540

### (3R)-2′-[6-amino-5-(trifluoromethyl)pyridin-3-yl]-N-[cyclopropyl(phenyl)methyl]-5′,6′-dihydrospiro[pyrrolidine-3,4′-pyrrolo[1,2-b]pyrazole]-1-carboxamide (Diastereomer 1 and Diastereomer 2)

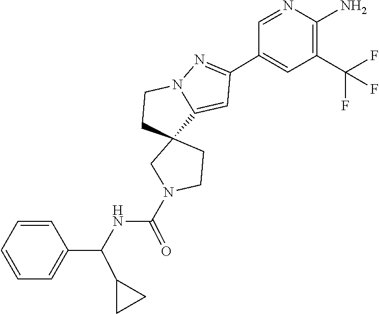 [SMILES: Nc1ncc(-c2cc3n(n2)CC[C@]32CCN(C(=O)NC(c3ccccc3)C3CC3)C2)cc1C(F)(F)F]

1-Cyclopropyl-1-phenylmethanamine (98.2 mg, 667 μmol) was dissolved in DMF (10 mL) under argon. CDI (108 mg, 667 μmol) and N,N-diisopropylethylamine (390 μL, 2.2 mmol) were added and stirred for 1 h at rt. 5-[(3R)-5′,6′-Dihydrospiro[pyrrolidine-3,4′-pyrrolo[1,2-b]pyrazol]-2′-yl]-3-(trifluoromethyl)pyridin-2-amine-hydrogen chloride (1/1) (200 mg, 556 μmol) was added and the mixture was stirred for 2 h at room temperature. The reaction mixture was concentrated, diluted with water and extracted with dichloromethane. The combined organic phases were concentrated. The residue was purified by column chromatography (silica gel, ethyl acetate/methanol (0-10%) gradient) to give 222 mg (80% yield) of the title compound.

LC-MS (Method 1): Rₜ=1.13 min; MS (ESIpos): m/z=497 [M+H]⁺

¹H NMR (DMSO-de, 400 MHz): δ (ppm) 8.55-8.59 (m, 1H), 8.00 (s, 1H), 7.36-7.44 (m, 2H), 7.25-7.33 (m, 2H), 7.16-7.23 (m, 1H), 6.65 (d, 1H), 6.55 (s, 2H), 6.43 (s, 1H), 4.19 (t, 2H), 4.06 (t, 1H), 3.52-3.62 (m, 1H), 3.40-3.51 (m, 3H), 2.52-2.57 (m, 2H), 2.01-2.14 (m, 2H), 1.14-1.27 (m, 1H), 0.42-0.54 (m, 2H), 0.27-0.40 (m, 2H).

### Intermediate 541

### (3S)-2′-[6-amino-5-(trifluoromethyl)pyridin-3-yl]-N-[cyclopropyl(phenyl)methyl]-5′,6′-dihydrospiro[pyrrolidine-3,4′-pyrrolo[1,2-b]pyrazole]-1-carboxamide (Diastereomer 1 and Diastereomer 2)

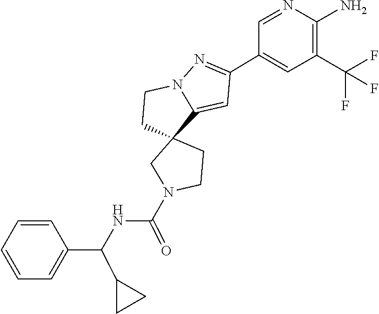 [SMILES: Nc1ncc(-c2cc3n(n2)CC[C@@]32CCN(C(=O)NC(c3ccccc3)C3CC3)C2)cc1C(F)(F)F]

1-Cyclopropyl-1-phenylmethanamine (98.2 mg, 667 μmol) was dissolved in DMF (10 mL). CDI (108 mg, 667 μmol) and N,N-diisopropylethylamine (390 μL, 2.2 mmol) were added and stirred for 1 h at room temperature. 5-[(3S)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (200 mg, 556 μmol) was added and the mixture was stirred for 2 h at room temperature. The reaction mixture was concentrated, diluted with water and extracted with dichloromethane. The combined organic layers were concentrated. The residue was purified by column chromatography (silica gel, ethyl acetate/methanol (0-10%) gradient) to give 227 mg (82.24% yield) of the title compound.

LC-MS (Method 1): Rₜ=1.13 min; MS (ESIpos): m/z=497 [M+H]⁺

¹H NMR (DMSO-d₆, 400 MHz): δ (ppm) 8.57 (s, 1H), 8.00 (s, 1H), 7.37-7.43 (m, 2H), 7.25-7.32 (m, 2H), 7.15-7.23 (m, 1H), 6.65 (d, 1H), 6.55 (s, 2H), 6.43 (s, 1H), 4.19 (t, 2H), 4.03-4.09 (m, 1H), 3.52-3.61 (m, 1H), 3.40-3.50 (m, 3H), 2.52-2.57 (m, 2H), 2.01-2.13 (m, 2H), 1.15-1.26 (m, 1H), 0.43-0.54 (m, 2H), 0.28-0.40 (m, 2H).

Oxidation

### Intermediate 542

### 1-(1H-imidazol-2-yl)-2-methylpropan-1-one

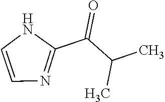

1-(1H-Imidazol-2-yl)-2-methylpropan-1-ol hydrogen chloride (1/1) (200 mg, 1.13 mmol) was dissolved in THF (11 mL). Mangan(IV)oxide (1.48 g, 17.0 mmol) was added and the mixture was stirred for 16 h at room temperature. The reaction mixture was filtered over celite and washed with ethyl acetate. The filtrate was concentrated to give 153 mg (98% purity, 96% yield) of the title compound.

LC-MS (Method 1): Rₜ=0.62 min; MS (ESIpos): m/z=139 [M+H]⁺

¹H-NMR (400 MHz, DMSO-d6) δ[ppm]: 1.109 (0.56), 1.114 (16.00), 1.126 (0.62), 1.131 (15.23), 2.518 (0.43), 3.700 (1.04), 3.717 (1.44), 3.734 (0.99), 7.171 (1.86), 7.174 (1.24), 7.412 (1.11), 7.414 (1.19), 7.418 (1.20), 7.420 (1.11).

### Intermediate 543

### 2-(1-chloro-2,2-dimethylpropyl)-1H-imidazol-3-ium chloride

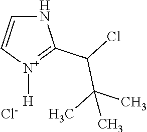

1-(1H-Imidazol-2-yl)-2,2-dimethylpropan-1-ol (60.0 mg, 389 μmol) was dissolved in dichloromethane (5 mL) and cooled to 0° C. Thionylchloride (57 μL, 780 μmol) was added and the mixture was stirred for 2 h at room temperature. The reaction mixture was concentrated to give 85.0 mg (104% yield) of the title compound which was used without further purification in the next step.

Acid Chloride

### Intermediate 544

### 3,3-difluorocyclobutane-1-carbonyl chloride

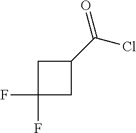

3,3-Difluorocyclobutane-1-carboxylic acid (1.00 g, 7.35 mmol) was dissolved in dichloromethane (20 mL) and DMF (20 μL, 0.26 mmol). Ethanedioyl dichloride (640 μL, 7.35 mmol) was added dropwise and the mixture was stirred for 1 h at room temperature. The reaction mixture was concentrated under reduce pressure (800 mbar) and used in the next step without further purification.

SEM-Protection

### Intermediate 545

### 1-{[2-(trimethylsilyl)ethoxy]methyl}-1H-imidazole

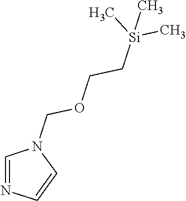

1H-Imidazole (3.00 g, 44.1 mmol) was dissolved in DMF (30 mL), cooled to 0° C. and sodium hydride (2.64 g, 60% purity, 66.1 mmol) was added. The mixture was stirred for 1 h at 0° C. and [2-(chloromethoxy)ethyl](trimethyl)silane (8.08 g, 48.5 mmol) was added at 0° C. and stirred over night to reach rt. The reaction mixture was diluted with water and extracted with ethyl acetate. The combined organic layers were washed with brine, dried over a hydrophobic filter and concentrated. The residue was purified by column chromatography (silica gel, hexane/ethyl acetate (0-100%) and ethyl acetate/ethanol (0-100%) gradient) to give 7.10 g (96% purity, 78% yield) of the title compound.

LC-MS (Method 1): Rₜ=1.10 min; MS (ESIpos): m/z=199 [M+H]⁺

¹H-NMR (400 MHz, DMSO-d6) δ[ppm]: −0.150 (0.42), 0.016 (0.54), 0.035 (1.86), 0.147 (0.41), 0.853 (4.10), 0.859 (0.53), 0.861 (0.44), 0.872 (4.30), 0.883 (0.40), 0.885 (0.54), 0.892 (4.27), 2.566 (0.45), 2.776 (1.13), 2.778 (1.08), 2.936 (1.33), 3.472 (4.55), 3.479 (0.49), 3.491 (3.46), 3.493 (4.64), 3.506 (0.47), 3.513 (4.55), 5.362 (16.00), 6.972 (2.70), 6.974 (4.35), 6.977 (2.71), 7.302 (3.02), 7.305 (5.56), 7.308 (2.88), 7.813 (2.59), 7.816 (4.08), 7.818 (2.52).

### Intermediate 546

### 1-{[2-(trimethylsilyl)ethoxy]methyl}-1H-1,2,4-triazole

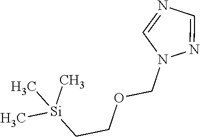

4H-1,2,4-Triazole (500 mg, 7.24 mmol) was provided in acetonitrile (32 mL). N,N-Diisopropylethylamine (1.9 mL, 10.9 mmol) and [2-(chloromethoxy)ethyl](trimethyl)silane (1.4 mL, 7.96 mmol) were added and the mixture was stirred for 19 h at room temperature. The reaction mixture was poured into water and extracted with ethyl acetate. The combined organic layers were washed with half concentrated aqueous sodium chloride solution, dried over a hydrophobic filter and concentrated. The residue was purified by column chromatography (silica gel, hexane/ethyl acetate gradient) to give 1230 mg (85% yield) of the title compound.

¹H-NMR (400 MHz, DMSO-d6) δ[ppm]: −0.150 (0.42), 0.010 (1.99), 0.037 (1.19), 0.147 (0.42), 0.866 (4.06), 0.874 (0.57), 0.887 (4.33), 0.900 (0.55), 0.907 (4.40), 2.571 (0.68), 2.575 (0.47), 3.588 (4.55), 3.595 (0.56), 3.606 (3.44), 3.609 (4.53), 3.621 (0.53), 3.628 (4.66), 5.558 (16.00), 8.095 (6.25), 8.766 (7.30).

### Intermediate 547

### 1-(1-{[2-(trimethylsilyl)ethoxy]methyl}-1H-imidazol-2-yl)propan-1-one

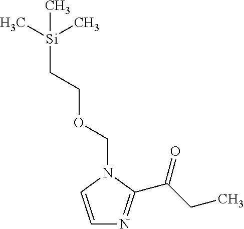 [SMILES: CCC(=O)c1nccn1COCC[Si](C)(C)C]

2-Propanoyl-1H-imidazol-1-ium chloride (300 mg, 1.87 mmol, CAS 1314916-37-4), [2-(chloromethoxy)ethyl](trimethyl)silane (330 μL, 1.9 mmol) and N,N-diisopropylethylamine (810 μL, 4.7 mmol) were dissolved in dichloromethane (4.8 mL) and stirred overnight at room temperature. The reaction mixture was diluted with water and extracted with dichloromethane. The combined organic layers were washed with brine, dried over sodium sulfate, filtered and concentrated. The residue was purified by column chromatography (silica gel, hexane/ethyl acetate (0-35%) gradient) to give 339 mg (98% purity, 70% yield) of the title compound.

LC-MS (Method 1): Rₜ=1.28 min; MS (ESIpos): m/z=255 [M+H]⁺

¹H-NMR (400 MHz, CHLOROFORM-d) 5 [ppm]: 0.025 (0.49), 0.026 (1.37), 0.041 (0.95), 0.045 (1.77), 0.929 (3.98), 0.936 (0.53), 0.947 (2.49), 0.949 (3.30), 0.952 (2.35), 0.963 (0.56), 0.970 (4.18), 1.201 (7.06), 1.219 (15.87), 1.238 (7.72), 3.156 (2.49), 3.175 (7.90), 3.193 (7.69), 3.211 (2.28), 3.565 (4.65), 3.572 (0.56), 3.583 (2.66), 3.586 (3.46), 3.588 (2.68), 3.599 (0.56), 3.606 (4.52), 5.811 (16.00), 7.205 (4.61), 7.207 (4.68), 7.286 (6.23), 7.309 (4.53), 7.311 (4.46).

### Intermediate 548

### 1-{[2-(trimethylsilyl)ethoxy]methyl}-1H-1,2,4-triazole

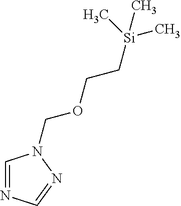

4H-1,2,4-Triazole (3.00 g, 43.4 mmol) was dissolved in DMF (20 mL), cooled to 0° C. and sodium hydride (2.61 g, 60% purity, 65.2 mmol) was added. The mixture was stirred for 1 h at 0° C., [2-(chloromethoxy)ethyl](trimethyl)silane (8.69 g, 52.1 mmol) was added and stirred over night at room temperature. The reaction mixture was diluted with water and extracted with ethyl acetate. The combined organic layers were washed with brine, dried over a hydrophobic filter and concentrated. The crude product was purified by column chromatography (silica gel, hexane/ethyl acetate (0-100%) and ethyl acetate/ethanol (0-100%) gradient) to give 5.30 g (61% yield) of the title compound.

LC-MS (Method 1): Rₜ=1.03 min; MS (ESIpos): m/z=200 [M+H]⁺

¹H-NMR (400 MHz, DMSO-d6) δ[ppm]: −0.183 (0.44), −0.054 (0.47), −0.047 (0.46), −0.041 (3.46), −0.025 (3.91), −0.018 (0.56), −0.011 (0.47), −0.008 (0.55), 0.008 (0.56), 0.013 (0.57), 0.018 (0.46), 0.113 (0.43), 0.834 (4.27), 0.841 (0.58), 0.854 (4.42), 0.866 (0.60), 0.874 (4.21), 3.555 (4.73), 3.562 (0.65), 3.576 (4.65), 3.588 (0.65), 3.595 (4.41), 5.525 (16.00), 5.535 (0.46), 8.061 (6.13), 8.732 (7.46).

### Intermediate 549

### (rac)-1-(1-propyl-1H-pyrazol-5-yl)ethan-1-ol

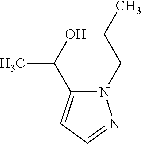

1-Propyl-1H-pyrazole-5-carbaldehyde (300 mg, 2.17 mmol) was dissolved in diethylether (20 mL) and cooled to 0° C. Methyl magnesium chloride (1.4 ml, 3.0 M, 4.3 mmol) was added and the mixture was stirred for 1 h at 0° C. A few drops of a saturated ammonium chloride solution were added and stirred for 10 min. The mixture was dried over sodium sulfate, filtered and concentrated to give 355 mg (87% purity, 92% yield) of the title compound.

LC-MS (Method 1): Rₜ=0.64 min; MS (ESIpos): m/z=155 [M+H]⁺

¹H-NMR (400 MHz, DMSO-d6) δ[ppm]: 0.798 (0.44), 0.826 (7.33), 0.844 (16.00), 0.863 (7.43), 1.072 (0.86), 1.089 (2.02), 1.107 (0.98), 1.399 (15.76), 1.415 (14.54), 1.716 (0.56), 1.734 (2.79), 1.741 (1.14), 1.753 (5.87), 1.758 (3.31), 1.762 (0.74), 1.767 (1.26), 1.771 (5.31), 1.789 (2.49), 1.808 (0.51), 3.372 (1.00), 3.389 (0.83), 3.583 (1.03), 3.585 (0.56), 3.589 (0.53), 3.593 (0.73), 3.599 (2.33), 3.606 (0.72), 3.608 (0.43), 3.610 (0.49), 3.614 (0.57), 3.616 (0.89), 3.982 (0.51), 4.000 (0.99), 4.016 (1.97), 4.034 (3.97), 4.038 (1.99), 4.052 (1.92), 4.057 (3.83), 4.076 (1.79), 4.091 (1.04), 4.109 (0.49), 4.786 (1.52), 4.800 (2.16), 4.817 (1.56), 4.832 (0.41), 5.234 (7.16), 5.248 (6.28), 6.110 (4.11), 6.115 (4.29), 7.293 (4.38), 7.298 (4.45).

### Intermediate 550

### tert-butyl (2-{(3R)-2′-[6-amino-5-(trifluoromethyl)pyridin-3-yl]-5′,6′-dihydrospiro[pyrrolidine-3,4′-pyrrolo[1,2-b]pyrazol]-1-yl}-2-oxoethyl)methylcarbamate

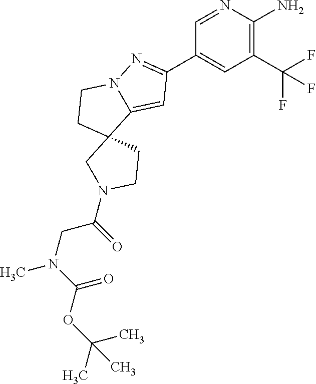 [SMILES: CN(CC(=O)N1CC[C@@]2(CCn3nc(-c4cnc(N)c(C(F)(F)F)c4)cc32)C1)C(=O)OC(C)(C)C]

5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (100 mg, 278 μmol) and N-(tert-butoxycarbonyl)-N-methylglycine (52.6 mg, 278 μmol, CAS 13734-36-6) were dissolved in DMF (1 mL). N,N-diisopropylethylamine (290 μL, 1.7 mmol) and propane phosphonic anhydride (560 μL, 50% purity, 830 μmol) were added and the mixture was stirred for 1 h at room temperature. The reaction mixture was diluted with water and extracted with ethyl acetate. The combined organic layers were dried over a hydrophobic filter and concentrated. The residue was purified by preparative HPLC to give 43.5 mg (100% purity, 32% yield) of the title compound.

LC-MS (Method 1): Rₜ=1.08 min; MS (ESIpos): m/z=495 [M+H]⁺

¹H-NMR (400 MHz, DMSO-d6) δ[ppm]: 1.318 (13.35), 1.338 (15.27), 1.392 (16.00), 1.404 (15.99), 2.048 (1.29), 2.056 (1.71), 2.075 (10.89), 2.084 (4.83), 2.131 (1.18), 2.148 (2.54), 2.165 (1.22), 2.518 (3.99), 2.523 (3.03), 2.563 (3.70), 2.577 (2.12), 2.778 (4.75), 2.801 (5.94), 2.812 (5.97), 2.841 (5.64), 3.480 (0.49), 3.509 (3.36), 3.519 (1.98), 3.544 (1.32), 3.566 (1.18), 3.575 (1.20), 3.598 (1.11), 3.616 (1.76), 3.642 (0.80), 3.665 (0.42), 3.684 (0.81), 3.708 (0.55), 3.859 (0.43), 3.893 (1.80), 3.947 (1.00), 3.981 (1.22), 4.022 (1.04), 4.048 (1.93), 4.090 (0.77), 4.172 (1.01), 4.185 (2.54), 4.189 (2.64), 4.202 (4.00), 4.219 (2.12), 6.458 (2.11), 6.474 (1.87), 6.521 (2.65), 6.528 (2.72), 6.546 (6.20), 7.979 (1.08), 8.003 (1.60), 8.021 (1.16), 8.567 (1.05), 8.587 (2.04).

### Intermediate 551

### tert-butyl [(2R)-1-{(3R)-2′-[6-amino-5-(trifluoromethyl)pyridin-3-yl]-5′,6′-dihydrospiro[pyrrolidine-3,4′-pyrrolo[1,2-b]pyrazol]-1-yl}-1-oxopropan-2-yl]methylcarbamate

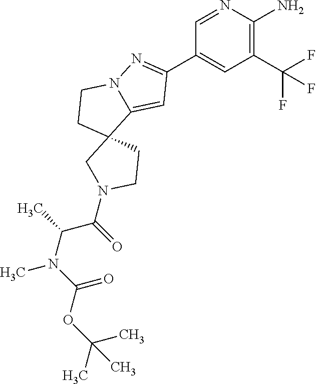 [SMILES: C[C@H](C(=O)N1CC[C@@]2(CCn3nc(-c4cnc(N)c(C(F)(F)F)c4)cc32)C1)N(C)C(=O)OC(C)(C)C]

5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (100 mg, 278 μmol) and N-(tert-butoxycarbonyl)-N-methyl-D-alanine (56.5 mg, 278 μmol, CAS 19914-38-6) were dissolved in DMF. N,N-Diisopropylethylamine (290 μL, 1.7 mmol) and propane phosphonic anhydride (265 mg, 834 μmol) were added and the mixture was stirred for 1 h at rt. The reaction mixture was diluted with water and extracted with ethyl acetate. The combined organic layers were dried over a hydrophobic filter and concentrated. The residue was purified by preparative HPLC to give 29.3 mg (100% purity, 21% yield) of the title compound.

LC-MS (Method 1): Rₜ=1.13 min; MS (ESIpos): m/z=509 [M+H]⁺

¹H-NMR (400 MHz, DMSO-d6) δ[ppm]: 1.122 (2.30), 1.139 (2.48), 1.181 (2.08), 1.196 (3.14), 1.227 (16.00), 1.258 (8.16), 1.409 (10.61), 1.420 (11.99), 1.751 (0.82), 2.045 (1.21), 2.062 (2.21), 2.075 (2.65), 2.084 (1.46), 2.151 (1.27), 2.318 (0.61), 2.323 (1.35), 2.327 (1.94), 2.332 (1.40), 2.336 (0.62), 2.518 (8.82), 2.523 (6.68), 2.576 (1.50), 2.641 (4.91), 2.660 (1.10), 2.665 (1.78), 2.669 (2.33), 2.673 (1.73), 2.678 (0.96), 2.706 (5.26), 2.726 (3.02), 3.476 (2.46), 3.505 (4.87), 3.555 (1.08), 3.572 (1.00), 3.590 (1.55), 3.606 (1.00), 3.620 (0.89), 3.637 (0.63), 3.748 (0.57), 4.126 (0.58), 4.184 (2.53), 4.200 (2.24), 4.801 (0.63), 4.818 (0.62), 4.896 (0.40), 6.409 (1.68), 6.495 (0.97), 6.540 (10.61), 7.992 (1.51), 8.009 (3.71), 8.013 (3.75), 8.568 (1.48), 8.594 (2.93), 8.599 (2.73).

### Intermediate 552

### (rac)-1-(pyrimidin-4-yl)ethan-1-ol

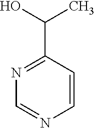

1-(Pyrimidin-4-yl)ethan-1-one (466 mg, 3.82 mmol) was dissolved in methanol (9.3 mL) and cooled to 0° C. Sodium borohydride (289 mg, 7.63 mmol) was added and allowed to reach rt within 2 h. The reaction mixture was diluted with brine and extracted with ethyl acetate and ethyl acetate/methanol 9:1. The combined organic layers were dried and concentrated. The residue was purified by column chromatography (silica gel, dichloromethane/methanol (0-10%) gradient) to give 500 mg (105% yield) of the title compound.

¹H NMR (400 MHz, DMSO-d) δ ppm=1.36 (d, 3H), 4.67 (qd, 1H), 5.62 (d, 1H), 7.59-7.62 (m, 1H), 8.77 (d, 1H), 9.07 (d, 1H).

Acylation

### Intermediate 553

### cyclobutyl(1-{[2-(trimethylsilyl)ethoxy]methyl}-1H-imidazol-2-yl)methanone

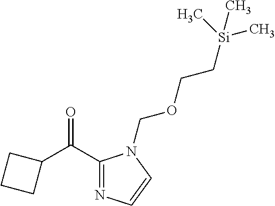 [SMILES: C[Si](C)(C)CCOCn1ccnc1C(=O)C1CCC1]

1-{[2-(Trimethylsilyl)ethoxy]methyl}-1H-imidazole (750 mg, 3.78 mmol, Intermediate 545) was dissolved in THF (38 mL) and cooled to −78° C. N-Butyllithium (1.8 mL, 2.5 M, 4.5 mmol) was added and stirred for 1 h at −78° C. A solution of cyclobutanecarbonyl chloride (470 μL, 4.2 mmol) in THF (2 mL) was added at −78° C. and the reaction mixture was stirred and allowed to reach rt over night. The reaction mixture was diluted with saturated sodium bicarbonate solution and extracted with ethyl acetate. The organic phase was washed with a saturated sodium bicarbonate solution, water and brine, dried over a hydrophobic filter and concentrated. The residue was purified by column chromatography (silica gel, hexane/ethyl acetate (0-100%) and ethyl acetate/ethanol (0-25%) gradient) to give 292 mg (90% purity, 25% yield) of the title compound.

LC-MS (Method 1): Rₜ=1.42 min; MS (ESIpos): m/z=281 [M+H]⁺

¹H-NMR (400 MHz, DMSO-d6) δ[ppm]: −0.150 (0.46), 0.016 (0.87), 0.019 (0.55), 0.028 (2.69), 0.035 (15.44), 0.040 (1.74), 0.045 (11.20), 0.051 (4.28), 0.054 (1.40), 0.058 (1.16), 0.061 (1.25), 0.066 (0.68), 0.068 (0.45), 0.146 (0.47), 0.878 (4.31), 0.885 (0.92), 0.897 (5.16), 0.905 (0.92), 0.911 (1.00), 0.918 (4.66), 0.925 (0.91), 0.931 (0.67), 0.945 (0.69), 1.313 (0.64), 1.339 (1.00), 1.818 (0.40), 1.833 (0.67), 1.837 (0.62), 1.840 (0.99), 1.843 (1.06), 1.851 (0.87), 1.854 (1.01), 1.857 (0.96), 1.859 (0.92), 1.862 (0.89), 1.867 (1.20), 1.869 (1.12), 1.873 (0.60), 1.876 (0.75), 1.879 (0.94), 1.882 (0.78), 1.889 (0.74), 1.892 (0.72), 1.900 (0.44), 1.902 (0.42), 2.021 (0.45), 2.035 (0.41), 2.044 (1.13), 2.065 (1.90), 2.071 (0.85), 2.088 (1.33), 2.091 (1.30), 2.108 (0.83), 2.115 (1.01), 2.135 (0.68), 2.159 (0.79), 2.168 (0.48), 2.180 (0.91), 2.184 (0.72), 2.190 (0.79), 2.196 (0.65), 2.202 (0.96), 2.204 (0.99), 2.207 (0.74), 2.214 (0.96), 2.218 (1.33), 2.222 (1.65), 2.226 (1.55), 2.229 (1.49), 2.232 (2.44), 2.239 (2.48), 2.243 (2.21), 2.249 (1.96), 2.254 (3.66), 2.256 (3.84), 2.264 (1.63), 2.271 (1.27), 2.277 (3.62), 2.281 (2.58), 2.284 (1.36), 2.298 (1.32), 2.301 (1.22), 2.305 (1.10), 2.308 (1.06), 2.328 (0.47), 2.598 (1.55), 2.603 (1.05), 2.745 (0.40), 2.749 (0.49), 3.484 (0.40), 3.548 (4.68), 3.555 (0.59), 3.568 (5.07), 3.577 (0.60), 3.581 (0.64), 3.588 (4.28), 3.595 (0.42), 3.597 (0.47), 4.316 (1.21), 4.319 (1.24), 4.338 (1.77), 4.341 (1.80), 4.359 (1.12), 4.362 (1.16), 4.789 (0.42), 4.989 (1.24), 5.365 (0.70), 5.800 (16.00), 6.959 (0.55), 6.961 (0.53), 6.964 (0.50), 6.968 (0.58), 7.026 (0.62), 7.034 (0.44), 7.218 (5.94), 7.220 (5.91), 7.340 (0.78), 7.344 (0.69), 7.350 (0.56), 7.353 (0.54), 7.686 (0.75), 7.690 (0.80), 7.725 (6.88), 7.727 (6.81).

### Intermediate 554

### cyclopropyl(6-methoxypyridin-2-yl)methanone

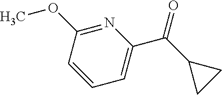

2-Bromo-6-methoxypyridine (910 mg, 4.84 mmol) was dissolved in diethylether (9.1 mL) and cooled to −70° C. N-Butyllithium (2.0 ml, 2.5 M, 5.1 mmol) was added dropwise over 30 min. The mixture was stirred for 1 h at −70° C. This solution was added dropwise to a solution of cyclopropanecarbonyl chloride (900 μL, 9.9 mmol) in THF (1.8 mL) over 10 min at −70° C. The reaction mixture was stirred for 4 h at −70° C. and allowed to reach rt over night. The reaction mixture was diluted with water and extracted twice with ethyl acetate. The combined organic layers were dried over sodium sulfate, filtered and concentrated. The residue was purified by column chromatography (silica gel, hexane/ethyl acetate (0-15%) gradient) to give 389 mg (70% purity, 32% yield) of the title compound.

LC-MS (Method 1): Rₜ=1.14 min; MS (ESIpos): m/z=178 [M+H]⁺

¹H-NMR (400 MHz, CHLOROFORM-d) 5 [ppm]: 0.915 (0.75), 0.926 (2.41), 0.934 (3.68), 0.944 (2.48), 0.946 (2.68), 0.954 (3.87), 0.962 (1.09), 0.964 (0.98), 1.053 (1.01), 1.055 (1.16), 1.063 (3.80), 1.071 (3.14), 1.073 (2.92), 1.074 (4.46), 1.082 (4.22), 1.091 (0.99), 1.093 (1.59), 1.102 (1.36), 1.110 (0.46), 1.219 (0.47), 1.226 (1.34), 1.230 (0.56), 1.238 (1.56), 1.246 (0.90), 1.583 (0.68), 1.594 (1.22), 1.603 (1.27), 1.606 (0.75), 1.615 (2.54), 1.623 (0.76), 1.626 (1.21), 1.635 (1.08), 1.646 (0.55), 3.487 (0.51), 3.495 (0.52), 3.506 (1.10), 3.518 (0.52), 3.526 (0.50), 3.899 (0.62), 3.932 (1.74), 3.954 (0.75), 4.043 (16.00), 6.943 (1.33), 6.946 (1.35), 6.963 (1.40), 6.966 (1.38), 7.637 (0.96), 7.639 (1.06), 7.655 (1.76), 7.658 (1.58), 7.694 (1.66), 7.712 (0.96), 7.714 (1.55), 7.732 (0.83).

### Intermediate 555

### (3,3-difluorocyclobutyl)(1-{[2-(trimethylsilyl)ethoxy]methyl}-1H-1,2,4-triazol-5-yl)methanone

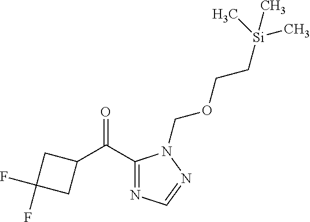 [SMILES: C[Si](C)(C)CCOCn1ncnc1C(=O)C1CC(F)(F)C1]

1-{[2-(Trimethylsilyl)ethoxy]methyl}-1H-1,2,4-triazole (1.23 g, 6.17 mmol, Intermediate 546) was provided in THF (20 mL) and cooled to −78° C. N-Butyllithium (4.2 mL, 1.6 M in hexane, 6.8 mmol) was added dropwise at −78° C. A solution of 3,3-difluorocyclobutane-1-carbonyl chloride (1.05 g, 6.79 mmol, Intermediate 544) in THF (20 mL) was added dropwise and the mixture was stirred over night at room temperature. The reaction mixture was diluted with a saturated aqueous sodium bicarbonate solution and extracted with ethyl acetate. The combined organic layers were washed with brine, dried over a hydrophobic filter and concentrated. The residue was purified by column chromatography (silica gel, hexane/ethyl acetate gradient) to give 285 mg (12% yield) of the title compound.

LC-MS (Method 1): Rₜ=1.39 min; MS (ESIpos): m/z=318 [M+H]⁺

¹H-NMR (400 MHz, DMSO-d6) δ[ppm]: −0.226 (0.07), −0.115 (0.03), −0.100 (0.04), −0.085 (0.61), −0.076 (16.00), −0.068 (0.64), −0.062 (0.32), −0.057 (1.65), −0.051 (0.19), −0.049 (0.19), −0.036 (0.07), −0.032 (0.08), −0.022 (0.08), −0.007 (0.02), 0.070 (0.07), 0.749 (0.05), 0.771 (0.04), 0.792 (0.05), 0.813 (0.56), 0.821 (0.09), 0.833 (0.67), 0.846 (0.09), 0.853 (0.59), 1.232 (0.03), 1.353 (0.01), 2.331 (0.05), 2.518 (0.26), 2.523 (0.17), 2.673 (0.06), 2.806 (0.03), 2.829 (0.03), 2.839 (0.14), 2.846 (0.09), 2.858 (0.23), 2.868 (0.16), 2.875 (0.28), 2.880 (0.35), 2.887 (0.23), 2.892 (0.26), 2.897 (0.33), 2.906 (0.23), 2.915 (0.32), 2.927 (0.15), 2.931 (0.17), 2.934 (0.23), 2.948 (0.05), 2.962 (0.06), 2.970 (0.02), 3.150 (0.04), 3.412 (0.03), 3.441 (0.10), 3.462 (0.05), 3.483 (0.05), 3.594 (0.63), 3.601 (0.08), 3.615 (0.71), 3.627 (0.08), 3.634 (0.61), 3.648 (0.03), 3.677 (0.05), 3.706 (0.02), 3.978 (0.03), 3.984 (0.03), 3.999 (0.09), 4.006 (0.10), 4.020 (0.12), 4.027 (0.13), 4.042 (0.08), 4.049 (0.09), 4.065 (0.02), 4.070 (0.02), 5.550 (0.15), 5.743 (0.03), 5.782 (2.10), 5.822 (0.01), 8.061 (0.17), 8.267 (1.54).

### Intermediate 556

### (1-methylcyclobutyl)(1-{[2-(trimethylsilyl)ethoxy]methyl}-1H-imidazol-2-yl)methanone

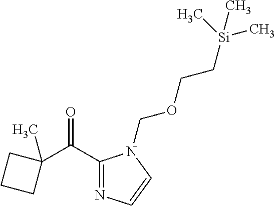 [SMILES: CC1(C(=O)c2nccn2COCC[Si](C)(C)C)CCC1]

1-{[2-(Trimethylsilyl)ethoxy]methyl}-1H-imidazole (596 mg, 3.01 mmol, Intermediate 545,) was dissolved in THF (9 mL) and cooled to −78° C. Butyllithium in hexane (1.4 mL, 2.5 M, 3.6 mmol) was added and stirred for 1 h at −78° C. A solution of N-methoxy-N,1-dimethylcyclobutane-1-carboxamide (520 mg, 3.31 mmol, Intermediate 574) in THF (2 mL) was added, the reaction mixture was allowed to reach rt and stirred over the weekend at rt. The reaction mixture was diluted with a saturated sodium bicarbonate solution and extracted three times with ethyl acetate. The combined organic layers were washed with water and brine, dried over a hydrophobic filter and concentrated. The residue was purified by column chromatography (silica gel, hexane/ethyl acetate (0-10%) gradient) to give 83.0 mg (94% purity, 9% yield) of the title compound.

LC-MS (Method 1): Rₜ=1.30 min; MS (ESIpos): m/z=296 [M+H]⁺

¹H-NMR (400 MHz, DMSO-d6) δ[ppm]: −0.064 (0.56), −0.056 (16.00), −0.047 (0.53), 0.822 (0.59), 0.842 (0.76), 0.862 (0.61), 1.196 (0.96), 1.208 (0.77), 1.618 (3.12), 3.082 (0.63), 3.491 (0.66), 3.510 (0.77), 3.530 (0.63), 3.643 (0.58), 5.733 (2.31), 7.179 (1.00), 7.182 (1.00), 7.631 (0.99), 7.634 (0.96).

### Intermediate 557

### cyclohexyl(1-{[2-(trimethylsilyl)ethoxy]methyl}-1H-imidazol-2-yl)methanone

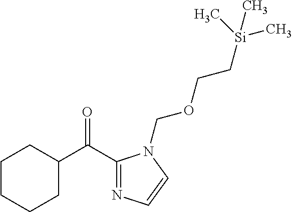 [SMILES: C[Si](C)(C)CCOCn1ccnc1C(=O)C1CCCCC1]

1-{[2-(Trimethylsilyl)ethoxy]methyl}-1H-imidazole (1.50 g, 7.56 mmol, Intermediate 545) was dissolved in THF (70 mL) and cooled to −78° C. N-Butyllithium in (3.6 mL, 2.5 M in hexane, 9.1 mmol) was added and stirred for 1 h at −78° C. A solution of cyclohexanecarbonyl chloride (1.22 g, 8.32 mmol) in THF (5 mL) was added at −78° C., the reaction mixture was allowed to warm up to rt and stirred over night at room temperature. The reaction mixture was washed with saturated sodium bicarbonate solution, water and brine, dried over a hydrophobic filter and concentrated. The residue was purified by column chromatography (silica gel, hexane/ethyl acetate (0-80%) gradient) to give 540 mg (90% purity, 21% yield) of the title compound.

LC-MS (Method 1): Rₜ=1.54 min; MS (ESIpos): m/z=310 [M+H]⁺

¹H-NMR (400 MHz, DMSO-d6) δ[ppm]: −0.077 (0.49), −0.069 (16.00), −0.061 (0.54), −0.009 (1.39), 0.805 (0.50), 0.824 (0.63), 0.845 (0.54), 1.194 (0.47), 1.211 (0.47), 1.243 (0.46), 1.280 (0.59), 1.317 (0.64), 1.345 (0.73), 1.666 (0.41), 1.800 (0.48), 1.813 (0.42), 1.828 (0.46), 2.008 (0.43), 3.352 (0.47), 3.465 (0.58), 3.486 (0.66), 3.505 (0.54), 5.715 (2.08), 7.181 (0.84), 7.184 (0.84), 7.671 (0.84), 7.674 (0.84).

### Intermediate 558

### cyclobutyl(1-{[2-(trimethylsilyl)ethoxy]methyl}-4H-1,2,4-triazol-5-yl)methanone

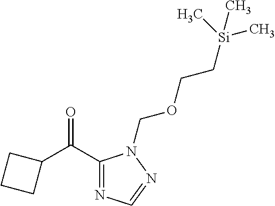 [SMILES: C[Si](C)(C)CCOCn1ncnc1C(=O)C1CCC1]

1-{[2-(Trimethylsilyl)ethoxy]methyl}-1H-1,2,4-triazole (750 mg, 3.76 mmol, Intermediate 548) was dissolved in THF (27 mL) and cooled to −78° C. N-butyllithium (1.8 mL, 2.5 M in hexane, 4.5 mmol) was added at −78° C. and stirred for 1 h. A solution of cyclobutanecarbonyl chloride (470 μL, 4.1 mmol) in THF (10 mL) was added, the mixture was allowed to warm up and stirred over night at room temperature. The reaction mixture was washed with saturated sodium bicarbonate solution, water and brine, dried over a hydrophobic filter and concentrated. The residue was purified by column chromatography (silica gel, hexane/ethyl acetate (0-100%) gradient) to give 435 mg (41% yield) of the title compound.

LC-MS (Method 1): Rₜ=1.43 min; MS (ESIpos): m/z=283 [M+H]⁺

¹H-NMR (400 MHz, DMSO-d6) δ[ppm]: −0.071 (0.50), −0.063 (13.09), −0.054 (0.50), −0.040 (0.70), −0.032 (16.00), −0.024 (1.85), −0.020 (1.14), −0.016 (1.84), 0.780 (0.72), 0.801 (0.94), 0.822 (1.10), 0.842 (0.59), 0.862 (0.54), 0.885 (0.56), 1.874 (0.53), 1.895 (0.76), 1.918 (0.70), 1.922 (0.42), 1.939 (0.49), 1.945 (0.55), 2.096 (0.45), 2.099 (1.57), 2.101 (0.44), 2.110 (0.44), 2.118 (1.10), 2.120 (1.95), 2.124 (0.99), 2.129 (0.64), 2.132 (0.58), 2.135 (0.43), 2.139 (0.77), 2.142 (1.32), 2.144 (1.19), 2.150 (0.43), 2.165 (0.44), 2.195 (0.41), 2.205 (0.74), 2.215 (0.54), 2.227 (1.28), 2.237 (0.53), 2.240 (0.52), 2.248 (1.26), 2.261 (0.48), 2.265 (0.59), 2.269 (0.71), 3.042 (0.46), 3.061 (0.54), 3.064 (0.54), 3.353 (1.56), 3.365 (1.14), 3.390 (0.47), 3.467 (0.44), 3.487 (0.82), 3.508 (1.07), 3.530 (0.48), 3.588 (0.54), 3.608 (0.57), 3.628 (0.47), 5.499 (1.27), 5.812 (1.64), 7.902 (0.67), 7.938 (0.42), 8.002 (1.32), 8.008 (2.91), 8.237 (1.07).

### Intermediate 559

### (1-methylcyclopropyl)(1-{[2-(trimethylsilyl)ethoxy]methyl}-1H-imidazol-2-yl)methanone

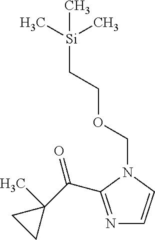 [SMILES: CC1(C(=O)c2nccn2COCC[Si](C)(C)C)CC1]

1-Methylcyclopropane-1-carboxylic acid (1.00 g, 9.99 mmol) was dissolved in dichloromethane (30 mL) and DMF (7 drops). Ethanedioyl dichloride (870 μL, 9.99 mmol) was added dropwise and the mixture was stirred for 2 h at room temperature. The reaction mixture was concentrated under reduce pressure (800 mbar) and used without further purification. 1-{[2-(Trimethylsilyl)ethoxy]methyl}-1H-imidazole (1.97 g, 9.95 mmol, Intermediate 545) was dissolved in THF (40 mL) and cooled to −75° C. N-Butyllithium (4.2 mL, 2.5 M in hexane, 10 mmol) was added and stirred for 1 h at −75° C. 1-Methylcyclopropane-1-carbonyl chloride (1.18 g, 9.95 mmol) was added dropwise at −75° C. and the mixture was stirred over the weekend at room temperature. The reaction mixture was diluted with a saturated aqueous sodium bicarbonate solution and extracted with ethyl acetate. The combined organic layers were washed with brine, dried over a hydrophobic filter and concentrated. The residue was purified by column chromatography (silica gel, dichloromethane/ethyl acetate gradient) to give 293 mg (10% yield) of the title compound.

¹H-NMR (400 MHz, DMSO-d6) δ[ppm]: −0.071 (0.57), −0.063 (16.00), −0.054 (0.56), 0.795 (0.62), 0.815 (0.76), 0.835 (0.63), 0.912 (0.97), 0.919 (0.99), 1.404 (2.99), 1.790 (0.67), 1.798 (0.66), 3.437 (0.68), 3.457 (0.77), 3.477 (0.64), 5.625 (2.38), 7.081 (0.98), 7.083 (0.94), 7.567 (0.99), 7.570 (0.95).

### Intermediate 560

### (3,3-difluorocyclobutyl)(1-{[2-(trimethylsilyl)ethoxy]methyl}-1H-imidazol-2-yl)methanone

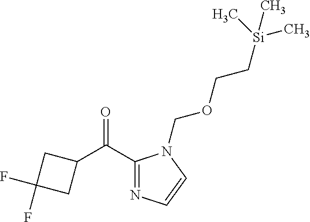 [SMILES: C[Si](C)(C)CCOCn1ccnc1C(=O)C1CC(F)(F)C1]

1-{[2-(Trimethylsilyl)ethoxy]methyl}-1H-imidazole (1.32 g, 6.68 mmol, Intermediate 545) was dissolved in THF (40 mL) and cooled to −78° C. N-Butyllithium (4.6 mL, 1.6 M in hexane, 7.3 mmol) was added and the mixture was stirred for 20 min at −78° C. 3,3-Difluorocyclobutane-1-carbonyl chloride (1.14 g, 7.34 mmol, Intermediate 544) was added dropwise at −78° C. and stirred for 1 h at −78° C. The reaction mixture was allowed to warm up to room temperature, quenched with a saturated sodium bicarbonate solution and extracted with ethyl acetate. The combined organic layers were washed with brine, dried over a hydrophobic filter and concentrated. The residue was purified by column chromatography (silica gel, hexane/ethyl acetate gradient) to give 285 mg (13% yield) of the title compound.

¹H-NMR (400 MHz, DMSO-d6) δ[ppm]: −0.062 (0.45), −0.054 (12.29), −0.045 (0.49), −0.037 (1.09), −0.026 (16.00), −0.023 (3.43), −0.018 (0.71), −0.007 (0.33), 0.011 (0.23), 0.709 (0.17), 0.724 (0.17), 0.735 (0.33), 0.751 (0.31), 0.761 (0.19), 0.777 (0.17), 0.831 (0.49), 0.851 (0.62), 0.871 (0.49), 2.823 (0.21), 2.842 (0.23), 2.853 (0.17), 2.858 (0.17), 2.882 (0.19), 3.156 (0.17), 3.171 (0.17), 3.196 (0.28), 3.222 (0.17), 3.237 (0.17), 3.515 (0.50), 3.535 (0.61), 3.555 (0.47), 5.015 (0.23), 5.041 (0.50), 5.072 (0.54), 5.098 (0.26), 5.339 (0.38), 5.740 (1.56), 6.240 (0.57), 6.904 (0.78), 6.907 (0.80), 7.234 (0.68), 7.237 (0.69), 7.264 (0.76), 7.267 (0.76), 7.752 (0.62), 7.754 (0.66).

### Intermediate 561

### cyclopentyl(1-{[2-(trimethylsilyl)ethoxy]methyl}-1H-imidazol-2-yl)methanone

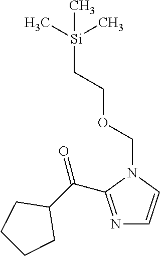 [SMILES: C[Si](C)(C)CCOCn1ccnc1C(=O)C1CCCC1]

1-{[2-(Trimethylsilyl)ethoxy]methyl}-1H-imidazole (1.00 g, 5.04 mmol, Intermediate 545) was dissolved in THF (10 mL) and cooled to −70° C. N-Butyllithium (2.2 mL, 2.5 M in hexane, 5.5 mmol) was added dropwise and stirred for 1 h at −70° C. Cyclopentanecarbonyl chloride (670 μL, 5.5 mmol) was added dropwise and the reaction mixture was allowed to reach room temperature and stirred for 17.5 h at room temperature. The reaction mixture was diluted with saturated aqueous sodium bicarbonate solution and extracted with ethyl acetate. The organic phase was washed with brine, dried over a hydrophobic filter and concentrated. The crude product was purified by column chromatography (silica gel, dichloromethane/ethanol gradient) followed by a second column chromatography (silica gel, hexane/ethyl acetate gradient) to give a first batch of 177 mg (10% yield) and a second batch of 65.0 mg (4% yield) of the title compound.

LC-MS (Method 1): Rₜ=1.49 min; MS (ESIpos): m/z=295 [M+H]⁺

¹H-NMR (400 MHz, DMSO-d6) δ[ppm]: −0.150 (0.48), 0.019 (0.49), 0.027 (4.31), 0.033 (0.48), 0.059 (5.72), 0.146 (0.48), 0.876 (4.26), 0.883 (1.31), 0.895 (5.12), 0.902 (0.85), 0.907 (0.78), 0.916 (4.53), 0.921 (0.61), 0.925 (0.65), 0.932 (0.42), 0.939 (0.58), 0.951 (0.58), 1.094 (0.67), 1.123 (0.66), 1.173 (1.55), 1.321 (1.31), 1.348 (2.40), 1.595 (0.48), 1.604 (0.50), 1.612 (0.69), 1.627 (0.54), 1.632 (0.60), 1.637 (0.53), 1.642 (0.49), 1.659 (0.96), 1.669 (1.44), 1.678 (2.52), 1.686 (4.02), 1.690 (3.82), 1.698 (3.76), 1.701 (3.97), 1.708 (3.10), 1.713 (2.65), 1.715 (2.45), 1.727 (1.22), 1.735 (1.03), 1.741 (1.42), 1.748 (1.08), 1.758 (1.32), 1.762 (1.24), 1.771 (1.48), 1.777 (1.40), 1.790 (1.83), 1.795 (1.08), 1.805 (1.09), 1.809 (1.41), 1.864 (0.44), 1.883 (0.41), 1.907 (0.74), 1.916 (1.10), 1.920 (1.19), 1.928 (1.17), 1.938 (1.48), 1.942 (1.44), 1.946 (1.36), 1.951 (1.18), 1.952 (1.13), 1.958 (1.07), 1.964 (0.83), 1.968 (1.02), 1.980 (0.46), 1.984 (0.40), 2.607 (1.09), 2.612 (0.72), 3.083 (0.66), 3.541 (4.90), 3.548 (0.66), 3.561 (5.42), 3.574 (0.59), 3.581 (4.68), 4.066 (0.57), 4.085 (1.26), 4.089 (1.15), 4.106 (1.91), 4.124 (1.11), 4.129 (0.86), 4.146 (0.44), 5.019 (0.77), 5.741 (0.55), 5.797 (16.00), 7.251 (6.53), 7.254 (6.53), 7.271 (0.54), 7.274 (0.48), 7.579 (0.43), 7.582 (0.50), 7.737 (6.96), 7.739 (6.95).

### Intermediate 562

### (1-methylcyclobutyl)(4-{[2-(trimethylsilyl)ethoxy]methyl}-1H-1,2,4-triazol-5-yl)methanone

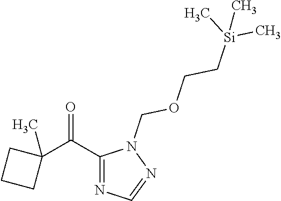 [SMILES: CC1(C(=O)c2ncnn2COCC[Si](C)(C)C)CCC1]

1-{[2-(Trimethylsilyl)ethoxy]methyl}-1H-1,2,4-triazole (596 mg, 2.99 mmol, Intermediate 548) was dissolved in THF (9 mL) and cooled to −78° C. N-Butyllithium (1.4 mL, 2.5 M in hexane, 3.6 mmol) was added and stirred for 1 h at −78° C. A solution of N-methoxy-N,1-dimethylcyclobutane-1-carboxamide (517 mg, 3.29 mmol, Intermediate 574) in THF (2 mL) was added dropwise at −78° C. and the reaction mixture was allowed to reach rt and stirred for 1 h at room temperature. The reaction mixture was diluted with saturated sodium bicarbonate solution and extracted three times with ethyl acetate. The combined organic phase was washed with water and brine, dried over a hydrophobic filter and concentrated. The crude product was purified by column chromatography (silica gel, hexane/ethyl acetate (0-10%) gradient) to give 364 mg (95% purity, 39% yield) of the title compound

LC-MS (Method 1): Rₜ=296.70 min; MS (ESIpos): m/z=2 [M+H]⁺

¹H-NMR (400 MHz, DMSO-d6) δ[ppm]: −0.073 (0.60), −0.065 (16.00), −0.057 (0.54), 0.820 (0.59), 0.840 (0.83), 0.859 (0.60), 1.616 (3.27), 3.580 (0.64), 3.600 (0.84), 3.619 (0.63), 5.808 (2.35), 8.262 (1.49).

SEM-Cleavage

### Intermediate 563

### cyclobutyl(1H-imidazol-2-yl)methanone

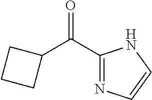 [SMILES: O=C(c1ncc[nH]1)C1CCC1]

Cyclobutyl(1-{[2-(trimethylsilyl)ethoxy]methyl}-1H-imidazol-2-yl)methanone (292 mg, 1.04 mmol, Intermediate 553) was dissolved in dichloromethane (2 mL). Trifluoroacetic acid (1.0 ml, 13 mmol) was added and the mixture was stirred over night at room temperature. The reaction mixture was concentrated and diluted with dichloromethane. The organic phase was extracted with saturated sodium bicarbonate solution, water and brine, dried and concentrated. The residue was purified by column chromatography (silica gel, hexane/ethyl acetate (0-100%) and ethyl acetate/ethanol (0-100%) gradient) to give 64.0 mg (100% purity, 41% yield) of the title compound.

LC-MS (Method 1): Rₜ=0.68 min; MS (ESIpos): m/z=151 [M+H]⁺

¹H-NMR (400 MHz, DMSO-d6) δ[ppm]: −0.081 (0.40), −0.045 (1.29), 0.818 (1.01), 0.836 (2.29), 0.854 (1.28), 1.154 (0.55), 1.172 (1.02), 1.190 (0.56), 1.232 (1.92), 1.264 (0.91), 1.282 (0.57), 1.749 (0.66), 1.753 (0.66), 1.760 (1.34), 1.763 (1.26), 1.772 (1.77), 1.774 (1.90), 1.779 (1.32), 1.782 (2.81), 1.786 (3.13), 1.790 (2.03), 1.794 (2.31), 1.798 (2.74), 1.801 (2.44), 1.805 (2.28), 1.809 (3.57), 1.812 (3.49), 1.815 (1.40), 1.819 (1.89), 1.822 (2.73), 1.824 (1.94), 1.832 (2.13), 1.834 (2.04), 1.842 (1.07), 1.844 (1.08), 1.899 (0.53), 1.921 (0.45), 1.957 (1.12), 1.980 (3.48), 1.984 (1.16), 1.988 (2.04), 2.001 (6.91), 2.007 (2.75), 2.024 (4.87), 2.028 (5.10), 2.045 (2.82), 2.051 (3.62), 2.071 (1.75), 2.106 (0.49), 2.113 (0.49), 2.126 (0.87), 2.130 (1.72), 2.133 (1.99), 2.135 (1.69), 2.140 (1.94), 2.142 (1.88), 2.146 (2.13), 2.152 (2.92), 2.157 (4.20), 2.159 (4.57), 2.160 (5.80), 2.166 (4.62), 2.170 (5.38), 2.174 (3.77), 2.177 (7.11), 2.180 (8.95), 2.182 (10.36), 2.187 (7.28), 2.192 (7.08), 2.204 (13.14), 2.212 (3.77), 2.214 (3.80), 2.224 (10.99), 2.228 (7.92), 2.245 (3.82), 2.249 (3.95), 2.252 (3.53), 2.254 (3.39), 2.272 (0.77), 2.276 (1.59), 2.337 (0.85), 2.518 (10.58), 2.523 (7.73), 2.679 (0.84), 4.141 (1.41), 4.143 (1.37), 4.162 (5.11), 4.165 (5.40), 4.184 (7.29), 4.187 (7.32), 4.205 (5.00), 4.207 (4.95), 4.226 (1.25), 4.228 (1.10), 5.725 (0.61), 6.243 (0.41), 7.136 (11.68), 7.139 (16.00), 7.142 (11.37), 7.401 (11.09), 7.404 (11.61), 7.407 (11.77), 7.410 (10.67), 13.200 (1.33).

### Intermediate 564

### (1H-imidazol-2-yl)(1-methylcyclobutyl)methanone

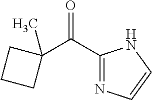

(1-Methylcyclobutyl)(1-{[2-(trimethylsilyl)ethoxy]methyl}-1H-imidazol-2-yl)methanone (83.0 mg, 282 μmol, Intermediate 556) was dissolved in dichloromethane (1.9 mL). Trifluoroacetic acid (720 μL, 9.3 mmol) was added and the mixture was stirred over night at room temperature. The reaction mixture was concentrated. The residue was diluted with dichloromethane and extracted with a saturated sodium bicarbonate solution and water. The organic phase was washed with brine, dried and concentrated. The crude product was purified by column chromatography (silica gel NH2, hexane/ethyl acetate (0-100%) and ethyl acetate/ethanol (0-100%) gradient) to give 34.0 mg (100% purity, 73% yield) of the title compound.

LC-MS (Method 1): Rₜ=0.79 min; MS (ESIneg): m/z=163 [M−H]⁻

¹H-NMR (400 MHz, DMSO-d6) δ[ppm]: 0.851 (0.19), 1.178 (1.79), 1.234 (1.13), 1.259 (0.18), 1.334 (0.42), 1.339 (0.54), 1.375 (0.29), 1.601 (16.00), 1.637 (0.19), 1.645 (0.27), 1.655 (0.25), 1.658 (0.40), 1.661 (0.31), 1.668 (0.95), 1.672 (0.35), 1.678 (0.49), 1.681 (0.51), 1.684 (0.45), 1.691 (0.56), 1.694 (0.82), 1.701 (0.34), 1.705 (0.53), 1.708 (0.31), 1.719 (0.36), 1.729 (0.28), 1.888 (0.72), 1.898 (0.58), 1.900 (0.45), 1.907 (0.48), 1.912 (1.05), 1.917 (1.15), 1.921 (1.43), 1.926 (0.82), 1.931 (0.37), 1.937 (0.77), 1.942 (0.96), 1.946 (0.99), 1.949 (0.87), 1.970 (0.43), 1.988 (0.66), 1.993 (0.35), 2.008 (0.68), 2.010 (0.63), 2.014 (0.70), 2.019 (0.21), 2.027 (0.30), 2.031 (0.52), 2.036 (0.70), 2.052 (0.21), 2.055 (0.29), 2.058 (0.43), 2.318 (0.16), 2.323 (0.34), 2.327 (0.49), 2.331 (0.37), 2.336 (0.17), 2.350 (0.35), 2.518 (2.99), 2.523 (1.62), 2.561 (0.45), 2.660 (0.16), 2.665 (0.35), 2.669 (0.49), 2.673 (0.35), 2.678 (0.16), 3.052 (0.91), 3.492 (0.50), 3.613 (0.82), 5.759 (0.23), 7.151 (2.24), 7.154 (3.08), 7.157 (2.22), 7.353 (2.08), 7.355 (2.23), 7.358 (2.24), 7.361 (2.13), 13.122 (0.27).

### Intermediate 565

### (3,3-difluorocyclobutyl)(1H-1,2,4-triazol-5-yl)methanone

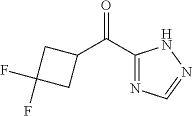 [SMILES: O=C(c1ncn[nH]1)C1CC(F)(F)C1]

(3,3-Difluorocyclobutyl)(1-{[2-(trimethylsilyl)ethoxy]methyl}-1H-1,2,4-triazol-5-yl)methanone (285 mg, 898 μmol, Intermediate 555) was provided in dichloromethane (20 mL). Trifluoroacetic acid (460 μL, 6.0 mmol) was added and the mixture was stirred for 2 h at room temperature. The reaction mixture was concentrated. The residue was purified by column chromatography (silica gel, dichloromethane/ethanol gradient) to give 136 mg (81% yield) of the title compound.

¹H-NMR (400 MHz, DMSO-d6) δ[ppm]: 1.035 (1.11), 1.052 (2.49), 1.070 (1.00), 2.331 (1.42), 2.337 (0.62), 2.518 (7.05), 2.523 (4.67), 2.673 (1.49), 2.678 (0.66), 2.819 (4.32), 2.844 (12.34), 2.857 (8.47), 2.865 (10.37), 2.882 (15.17), 2.903 (11.44), 3.929 (1.56), 3.937 (2.21), 3.949 (2.94), 3.959 (3.18), 3.970 (2.94), 3.980 (2.04), 3.991 (1.62), 5.546 (8.22), 5.565 (8.12), 5.759 (0.59), 7.256 (1.90), 7.275 (4.08), 7.294 (1.80), 8.203 (0.93), 8.783 (16.00), 8.812 (13.41), 14.735 (2.04).

### Intermediate 566

### 4-chloro-2-propanoyl-1H-imidazol-1-ium chloride

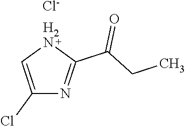

1-(4-Chloro-1-{[2-(trimethylsilyl)ethoxy]methyl}-1H-imidazol-2-yl)propan-1-one (85.0 mg, 294 μmol, Intermediate 576) was dissolved in THF (6.8 mL) and methanol (6.8 mL). Hydrochloric acid in 1,4-dioxane (3.4 mL, 4.0 M, 14 mmol) was added and the mixture was stirred over night at room temperature and 2 h at 50° C. The reaction mixture was allowed to cool down, concentrated to give 63.8 mg (75% purity, 83% yield) of the title compound which was used without further purification in the next step.

¹H-NMR (400 MHz, DMSO-d6) δ[ppm]: 1.046 (7.04), 1.064 (16.00), 1.083 (7.25), 1.347 (0.92), 1.496 (0.41), 1.513 (0.47), 1.533 (0.47), 1.723 (0.47), 1.743 (0.55), 1.760 (0.47), 2.518 (1.10), 2.523 (0.74), 2.923 (2.09), 2.941 (7.06), 2.959 (6.87), 2.977 (1.92), 3.201 (0.41), 3.336 (0.82), 3.620 (1.03), 3.636 (2.09), 3.654 (1.00), 7.550 (8.23).

### Intermediate 567

### cyclohexyl(1H-imidazol-2-yl)methanone

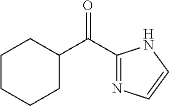 [SMILES: O=C(c1ncc[nH]1)C1CCCCC1]

Cyclohexyl(1-{[2-(trimethylsilyl)ethoxy]methyl}-1H-imidazol-2-yl)methanone (540 mg, 1.75 mmol, Intermediate 557) was dissolved in 1,4-dioxane. Hydrochloric acid in 1,4-dioxane (2.2 mL, 4.0 M, 8.8 mmol) was added and the mixture was stirred for 3 days at room temperature. The reaction mixture was concentrated and diluted with dichloromethane. The organic phase was washed with a saturated sodium bicarbonate solution, water and brine, dried and concentrated. The crude product was purified by column chromatography (silica gel, hexane/ethyl acetate (0-100%) and ethyl acetate/ethanol (0-100%) gradient) to give 96.0 mg (90% purity, 28% yield) of the title compound.

LC-MS (Method 1): Rₜ=0.88 min; MS (ESIpos): m/z=179 [M+H]⁺

1H-NMR (400 MHz, DMSO-d6) δ[ppm]: −0.078 (1.88), −0.055 (1.01), −0.045 (4.91), −0.040 (1.21), −0.038 (1.08), −0.029 (16.00), −0.021 (0.67), −0.010 (0.47), 0.015 (0.67), 0.032 (0.67), 0.039 (0.40), 0.864 (0.47), 1.167 (1.48), 1.178 (1.48), 1.185 (2.89), 1.195 (2.02), 1.203 (2.35), 1.209 (2.22), 1.218 (2.69), 1.226 (2.08), 1.245 (2.69), 1.293 (1.55), 1.299 (1.88), 1.306 (1.82), 1.332 (9.01), 1.344 (5.31), 1.355 (15.46), 1.384 (8.47), 1.416 (1.68), 1.669 (3.36), 1.697 (2.76), 1.701 (2.96), 1.752 (4.71), 1.760 (5.92), 1.766 (5.38), 1.773 (6.12), 1.780 (4.77), 1.838 (6.52), 1.857 (5.18), 2.000 (2.42), 2.349 (1.21), 2.690 (1.21), 3.464 (0.87), 3.472 (1.61), 3.481 (1.61), 3.493 (2.55), 3.501 (3.23), 3.509 (2.22), 3.520 (1.68), 3.529 (1.48), 4.030 (0.54), 4.047 (0.54), 5.202 (0.47), 5.228 (0.54), 5.699 (1.55), 6.813 (0.47), 6.866 (0.94), 6.869 (0.94), 7.171 (6.79), 7.206 (1.01), 7.210 (1.01), 7.419 (6.18), 13.179 (1.61).

### Intermediate 568

### cyclobutyl(4H-1,2,4-triazol-3-yl)methanone

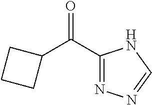 [SMILES: O=C(c1nnc[nH]1)C1CCC1]

Cyclobutyl(1-{[2-(trimethylsilyl)ethoxy]methyl}-4H-1,2,4-triazol-5-yl)methanone (435 mg, 1.55 mmol, Intermediate 558) was dissolved in dichloromethane (3.3 mL). Trifluoroacetic acid (1.5 mL, 19 mmol) was added and the mixture was stirred over night at room temperature. The reaction mixture was concentrated and diluted with dichloromethane. The organic phase was washed with saturated sodium bicarbonate, water and brine, dried and concentrated. The crude product was purified by column chromatography (silica gel, hexane/ethyl acetate (0-100%) and ethyl acetate/ethanol (0-100%) gradient) to give 40.7 mg (93% purity, 16% yield) of the title compound.

¹H-NMR (400 MHz, DMSO-d6) δ[ppm]: 0.855 (1.00), 0.873 (1.94), 0.878 (0.80), 0.891 (0.87), 1.154 (2.54), 1.173 (5.22), 1.190 (2.88), 1.232 (1.27), 1.282 (0.47), 1.352 (0.47), 1.784 (0.67), 1.799 (0.80), 1.816 (0.94), 1.825 (1.00), 1.827 (1.00), 1.840 (1.34), 1.843 (1.27), 1.847 (1.61), 1.850 (1.47), 1.852 (1.61), 1.855 (1.67), 1.858 (1.67), 1.863 (1.87), 1.874 (2.34), 1.877 (2.41), 1.886 (1.74), 1.888 (1.81), 1.896 (1.54), 1.900 (1.54), 1.908 (0.94), 1.912 (1.14), 1.916 (1.34), 1.938 (3.01), 1.943 (0.80), 1.960 (5.96), 1.966 (1.87), 1.982 (3.95), 1.988 (13.12), 2.003 (3.35), 2.009 (3.01), 2.022 (5.49), 2.030 (2.48), 2.042 (7.70), 2.061 (5.69), 2.081 (1.94), 2.088 (1.61), 2.097 (0.60), 2.105 (2.21), 2.109 (1.94), 2.126 (2.48), 2.130 (1.54), 2.146 (1.34), 2.164 (0.94), 2.183 (1.27), 2.192 (1.34), 2.205 (2.08), 2.215 (3.28), 2.218 (3.88), 2.222 (3.68), 2.231 (5.89), 2.237 (6.03), 2.240 (5.62), 2.252 (9.24), 2.255 (9.04), 2.261 (5.09), 2.276 (8.97), 2.279 (5.22), 2.296 (2.54), 2.300 (2.21), 2.306 (1.87), 2.318 (1.47), 2.323 (3.15), 2.327 (4.55), 2.332 (3.01), 2.336 (1.34), 2.518 (16.00), 2.523 (11.18), 2.665 (5.49), 2.669 (6.96), 2.673 (5.76), 2.687 (6.69), 2.704 (2.81), 2.711 (2.61), 3.049 (2.95), 3.053 (3.01), 3.057 (2.74), 3.069 (5.36), 3.072 (5.22), 3.088 (2.81), 3.092 (2.61), 3.164 (0.47), 3.185 (0.67), 3.209 (0.40), 3.362 (3.48), 3.385 (1.81), 3.405 (0.60), 4.000 (0.74), 4.017 (2.28), 4.035 (2.21), 4.053 (0.67), 4.128 (0.47), 4.241 (0.40), 5.388 (2.41), 5.406 (2.54), 5.759 (10.64), 6.186 (3.88), 6.960 (0.40), 6.978 (0.80), 6.998 (0.47), 8.303 (0.87), 8.518 (8.10), 8.524 (0.74), 8.752 (0.60), 8.915 (2.68), 13.935 (0.87).

### Intermediate 569

### (1H-imidazol-2-yl)(1-methylcyclopropyl)methanone

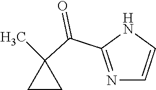

(1-Methylcyclopropyl)(1-{[2-(trimethylsilyl)ethoxy]methyl}-1H-imidazol-2-yl)methanone (293 mg, 1.04 mmol, Intermediate 559) was dissolved in dichloromethane (20 mL). Trifluoroacetic acid (1.1 mL, 14 mmol) was added and the mixture was stirred for 50 h at room temperature. Trifluoroacetic acid (1.1 mL, 14 mmol) was added and stirred for 23 h at 50° C. The reaction mixture was concentrated and the residue was diluted with ethyl acetate. The organic phase was washed with saturated aqueous sodium bicarbonate solution and brine, dried over a hydrophobic filter and concentrated to give 162 mg (98% yield) of the title compound which was used without further purification in the next step.

LC-MS (Method 1): Rₜ=0.71 min; MS (ESIpos): m/z=151 [M+H]⁺

¹H-NMR (400 MHz, DMSO-d6) δ[ppm]: −0.034 (0.59), 0.944 (1.22), 0.946 (1.35), 0.953 (4.93), 0.961 (5.16), 0.968 (1.33), 0.971 (1.25), 0.985 (0.31), 1.237 (0.16), 1.246 (0.16), 1.280 (0.33), 1.397 (16.00), 1.954 (1.17), 1.962 (3.65), 1.970 (3.57), 1.978 (1.09), 5.280 (0.17), 7.211 (0.21).

### Intermediate 570

### (3,3-difluorocyclobutyl)(1H-imidazol-2-yl)methanone

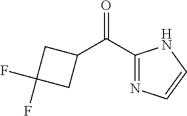 [SMILES: O=C(c1ncc[nH]1)C1CC(F)(F)C1]

(3,3-Difluorocyclobutyl)(1-{[2-(trimethylsilyl)ethoxy]methyl}-1H-imidazol-2-yl)methanone (892 mg, 2.82 mmol, Intermediate 560) was dissolved in dichloromethane (30 mL). Trifluoroacetic acid (0.8 mL, 10 mmol) was added and the mixture was stirred over night at room temperature. Trifluoroacetic acid (2 mL, 26 mmol) was added and the mixture was stirred for 72 h at room temperature. The reaction mixture was concentrated to give a first batch of crude product. (3,3-Difluorocyclobutyl)(1-{[2-(trimethylsilyl)ethoxy]methyl}-1H-imidazol-2-yl)methanone (218 mg, 0.69 mmol, Intermediate 560) was dissolved in dichloromethane (20 mL). Trifluoroacetic acid (0.5 mL, 6 mmol) was added and the mixture was stirred over night at room temperature. Trifluoroacetic acid (2 mL, 26 mmol) was added and the mixture was stirred for 72 h at room temperature. The reaction mixture was concentrated to give a second batch of crude product. The two batches were purified together by column chromatography (silica gel, dichloromethane/ethyl acetate gradient) to give 372 mg (57% yield) of the title compound.

LC-MS (Method 1): Rₜ=0.69 min; MS (ESIpos): m/z=187 [M+H]⁺

¹H-NMR (400 MHz, DMSO-d6) δ[ppm]: 1.154 (0.52), 1.172 (1.04), 1.190 (0.57), 1.232 (0.57), 1.907 (0.65), 1.987 (2.11), 2.331 (1.09), 2.518 (7.40), 2.523 (4.42), 2.673 (1.17), 2.794 (0.65), 2.809 (3.48), 2.834 (10.01), 2.848 (6.26), 2.855 (8.10), 2.872 (12.60), 2.894 (9.57), 3.967 (0.67), 3.973 (0.72), 3.988 (2.06), 3.995 (2.06), 4.010 (2.83), 4.017 (3.20), 4.031 (1.96), 4.039 (1.96), 4.053 (0.82), 4.060 (0.67), 4.592 (0.87), 4.673 (0.47), 5.682 (1.47), 7.179 (0.57), 7.181 (0.55), 7.384 (16.00), 7.650 (0.57), 7.653 (0.60).

### Intermediate 571

### cyclopentyl(1H-imidazol-2-yl)methanone

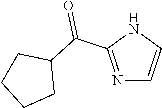 [SMILES: O=C(c1ncc[nH]1)C1CCCC1]

Cyclopentyl(1-{[2-(trimethylsilyl)ethoxy]methyl}-1H-imidazol-2-yl)methanone (242 mg, 678 μmol, Intermediate 561) was dissolved in dichloromethane (7.1 mL). Trifluoroacetic acid (710 μL, 9.3 mmol) was added and the mixture was stirred for 22 h at room temperature. The reaction mixture was concentrated. The residue was dissolved In ethyl acetate and washed with saturated aqueous sodium bicarbonate solution and brine. The organic phase was dried over a hydrophobic filter and concentrated. The crude product was purified by column chromatography (silica gel, hexane/ethyl acetate gradient) to give 54.8 mg (48% yield) of the title compound.

LC-MS (Method 1): Rₜ=0.80 min; MS (ESIpos): m/z=165 [M+H]⁺

¹H-NMR (400 MHz, DMSO-d6) δ[ppm]: 1.230 (0.84), 1.258 (0.67), 1.559 (1.01), 1.565 (1.18), 1.576 (2.36), 1.579 (2.86), 1.590 (5.05), 1.598 (7.07), 1.607 (11.45), 1.611 (14.15), 1.614 (13.98), 1.627 (15.33), 1.637 (7.92), 1.642 (8.93), 1.661 (4.88), 1.673 (4.55), 1.691 (4.38), 1.694 (4.21), 1.703 (5.56), 1.710 (6.06), 1.722 (7.75), 1.728 (5.39), 1.734 (4.21), 1.741 (5.89), 1.757 (1.85), 1.842 (2.53), 1.852 (4.55), 1.856 (5.22), 1.864 (5.22), 1.870 (6.06), 1.873 (6.91), 1.878 (6.57), 1.886 (5.05), 1.888 (5.05), 1.893 (5.05), 1.904 (4.55), 1.916 (1.85), 1.920 (1.52), 2.518 (4.88), 2.523 (3.20), 3.399 (1.01), 3.863 (2.53), 3.882 (5.73), 3.885 (5.39), 3.893 (0.84), 3.903 (8.76), 3.921 (5.05), 3.924 (4.21), 3.942 (2.02), 7.163 (16.00), 7.408 (14.99), 13.179 (1.85).

### Intermediate 572

### (1-methylcyclobutyl)(4H-1,2,4-triazol-3-yl)methanone

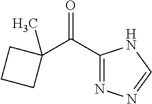

(1-Methylcyclobutyl)(1-{[2-(trimethylsilyl)ethoxy]methyl}-4H-1,2,4-triazol-5-yl)methanone (364 mg, 1.23 mmol, Intermediate 562) was dissolved in dichloromethane (8.4 mL). Trifluoroacetic acid (3.1 mL, 41 mmol) was added and the mixture was stirred over night at room temperature. The reaction mixture was concentrated and the residue was dissolved in dichloromethane. The organic phase was washed with saturated sodium bicarbonate solution, water and brine, dried and concentrated. The crude product was purified by column chromatography (silica gel, hexane/ethyl acetate (0-100%) and ethyl acetate/ethanol (0-100%) gradient) to give 67.0 mg (100% purity, 33% yield) of the title compound.

LC-MS (Method 2): Rₜ=0.75 min; MS (ESIneg): m/z=164 [M−H]⁻

¹H-NMR (400 MHz, DMSO-d6) δ[ppm]: 1.640 (16.00), 1.740 (0.86), 1.750 (0.45), 1.753 (0.53), 1.757 (0.41), 1.764 (0.52), 1.767 (0.83), 1.778 (0.47), 1.958 (0.74), 1.967 (0.57), 1.970 (0.45), 1.976 (0.43), 1.981 (1.02), 1.987 (1.06), 1.991 (1.43), 1.996 (0.91), 2.003 (0.57), 2.006 (0.70), 2.012 (0.96), 2.016 (0.88), 2.020 (0.81), 2.070 (0.66), 2.092 (0.70), 2.097 (0.70), 2.115 (0.53), 2.120 (0.74), 2.141 (0.43), 2.586 (2.79), 2.591 (1.95), 2.593 (1.78), 2.616 (0.49), 8.617 (0.57).

### Intermediate 573

### 1-(1H-pyrazol-3-yl)propan-1-one

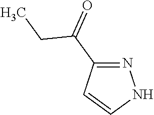

1-[1-(Oxan-2-yl)-1H-pyrazol-3-yl]propan-1-one (314 mg, 75% purity, 1.13 mmol, Intermediate 577) was dissolved in methanol. Hydrochloric acid (840 μL, 36% purity, 11 mmol) was added dropwise and the mixture was stirred for 3 h at room temperature. The reaction mixture was concentrated. The residue was diluted with a saturated sodium bicarbonate solution and extracted with ethyl acetate. The combined organic layers were dried over a hydrophobic filter and concentrated. The crude product was purified by column chromatography (silica gel, hexane/ethyl acetate (0-100%) gradient) to give 58.0 mg (41% yield) of the title compound.

¹H NMR (400 MHz, CHLOROFORM-d) δ ppm 1.23 (t, 3H), 2.99 (q, 2H), 6.84 (d, H), 7.64 (d, 1H), 10.94 (br d, 1H).

Weinreb Amide

### Intermediate 574

### N-methoxy-N,1-dimethylcyclobutane-1-carboxamide

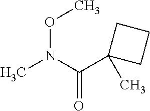

1-Methylcyclobutane-1-carboxylic acid (490 mg, 4.29 mmol) and N-methoxymethanamine hydrogen chloride (1/1) (461 mg, 4.72 mmol) were dissolved in DMF (4.5 mL). Triethylamine (1.8 mL, 13 mmol) and HATU (1.96 g, 5.15 mmol) were added and the mixture was stirred over night at room temperature. The reaction mixture was diluted with ethyl acetate and extracted with saturated sodium bicarbonate solution and water. The organic phase was washed with brine, dried and concentrated. The crude product was purified by column chromatography (silica gel, hexane/ethyl acetate (0-100%) and ethyl acetate/ethanol (0-95%) gradient) to give 520 mg (82% purity, 64% yield) of the title compound.

LC-MS (Method 1): Rₜ=0.83 min; MS (ESIpos): m/z=158 [M+H]⁺

¹H-NMR (400 MHz, DMSO-d6) δ[ppm]: 1.155 (0.19), 1.173 (0.41), 1.190 (0.20), 1.339 (7.87), 1.572 (0.19), 1.589 (0.19), 1.596 (0.45), 1.600 (0.23), 1.603 (0.25), 1.607 (0.19), 1.616 (0.21), 1.619 (0.28), 1.623 (0.49), 1.627 (0.19), 1.631 (0.28), 1.653 (0.18), 1.678 (0.50), 1.684 (0.45), 1.691 (0.25), 1.695 (0.21), 1.700 (0.53), 1.702 (0.47), 1.707 (0.78), 1.713 (0.50), 1.716 (0.30), 1.723 (0.44), 1.730 (0.70), 1.733 (0.28), 1.737 (0.46), 1.890 (0.34), 1.894 (0.16), 1.912 (0.45), 1.914 (0.37), 1.917 (0.30), 1.934 (0.32), 1.940 (0.43), 1.941 (0.25), 1.962 (0.24), 1.987 (0.61), 2.311 (0.35), 2.317 (0.23), 2.335 (0.87), 2.341 (0.66), 2.358 (0.65), 2.364 (0.80), 2.382 (0.18), 2.389 (0.25), 2.687 (0.35), 2.728 (0.59), 2.730 (0.67), 2.889 (0.75), 3.052 (16.00), 3.328 (1.82).

### Intermediate 575

### N-methoxy-N-methyl-1H-pyrazole-3-carboxamide

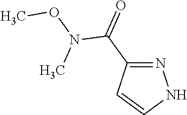

1H-Pyrazole-3-carboxylic acid (500 mg, 4.46 mmol) and N-methoxymethanamine (300 mg, 4.91 mmol) were dissolved DMF (7.5 mL). Triethylamine (1.9 mL, 13 mmol) and HATU (2.04 g, 5.35 mmol) were added and stirred for 2 h at room temperature. The reaction mixture was diluted with ethyl acetate. The organic phase was washed with sodium bicarbonate solution, water and brine, dried over a hydrophobic filter and concentrated. The crude product was purified by column chromatography (silica gel). The residue was dissolved in ethyl acetate and washed with ammonium chloride solution, water and brine. The organic phase was dried over a hydrophobic filter and concentrated to give 194 mg (28% yield) of the title compound.

LC-MS (Method 1): Rₜ=0.53 min; MS (ESIpos): m/z=156 [M+H]⁺

¹H-NMR (400 MHz, DMSO-d6) δ[ppm]: 1.153 (4.50), 1.171 (10.10), 1.189 (4.65), 2.518 (2.70), 2.523 (1.77), 2.687 (16.00), 2.728 (2.42), 2.888 (3.05), 3.065 (0.40), 3.084 (1.24), 3.095 (1.13), 3.102 (1.31), 3.112 (1.06), 3.119 (0.52), 3.276 (1.06), 3.355 (0.52), 3.697 (1.29), 3.725 (1.16).

### Intermediate 576

### 1-(4-chloro-1-{[2-(trimethylsilyl)ethoxy]methyl}-1H-imidazol-2-yl)propan-1-one

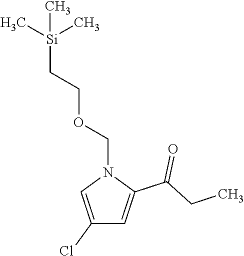 [SMILES: CCC(=O)c1cc(Cl)cn1COCC[Si](C)(C)C]

1-(1-{[2-(Trimethylsilyl)ethoxy]methyl}-1H-imidazol-2-yl)propan-1-one (150 mg, 590 μmol, Intermediate 547) was dissolved in acetonitrile (1.9 mL). N-Chlorsuccinimid (78.7 mg, 590 μmol) was added and the mixture was stirred for 1 h at room temperature and for 4 h at 60° C. The reaction mixture was allowed to cool down, diluted with a saturated sodium carbonate solution and extracted with ethyl acetate. The combined organic layers were dried over sodium sulfate, filtered and concentrated. The residue was purified by column chromatography (silica gel, hexane/ethyl acetate (0-25%) gradient) to give 87.8 mg (52% yield) of the title compound as a mixture of isomers of 4:1.

LC-MS (Method 1): Rₜ=1.47 min; MS (ESIpos): m/z=289 [M+H]⁺

¹H-NMR (400 MHz, CHLOROFORM-d) 5 [ppm]: −0.037 (0.53), −0.028 (16.00), −0.019 (0.94), −0.008 (2.28), 0.008 (2.28), 0.012 (0.53), 0.894 (0.72), 0.912 (0.47), 0.914 (0.63), 0.917 (0.53), 0.926 (2.85), 0.935 (0.91), 0.945 (1.67), 0.947 (2.15), 0.951 (1.56), 0.968 (2.87), 1.163 (5.38), 1.179 (1.95), 1.182 (11.92), 1.197 (3.24), 1.200 (5.63), 1.215 (1.39), 1.567 (7.15), 3.095 (1.67), 3.102 (0.46), 3.113 (5.53), 3.121 (1.36), 3.132 (5.40), 3.139 (1.36), 3.150 (1.56), 3.158 (0.43), 3.565 (3.33), 3.574 (0.90), 3.583 (1.84), 3.586 (2.31), 3.589 (1.96), 3.591 (0.70), 3.595 (0.74), 3.596 (0.58), 3.600 (0.48), 3.607 (3.23), 3.615 (0.85), 5.755 (12.09), 5.851 (2.86), 7.107 (1.53), 7.210 (6.08).

### Intermediate 577

### 1-[1-(oxan-2-yl)-1H-pyrazol-3-yl]propan-1-one

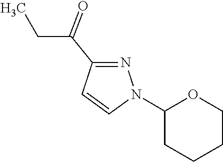

N-Methoxy-N-methyl-1-(oxan-2-yl)-1H-pyrazole-3-carboxamide (400 mg, 78% purity, 1.30 mmol, Intermediate 578) was dissolved in dry THF (4.4 mL) and cooled to 0° C. Ethyl magnesium bromide (530 μL, 3.2 M in THF, 1.7 mmol) was added slowly and the mixture was stirred for 1 h at 0° C. The reaction mixture was diluted with saturated ammonium chloride solution and extracted with ethyl acetate. The combined organic layers were concentrated to give 132 mg (48% yield) of the title compound.

LC-MS (Method 1): Rₜ=1.00 min; MS (ESIpos): m/z=209 [M+H]⁺

¹H-NMR (400 MHz, DMSO-d6) δ[ppm]: 1.045 (7.25), 1.063 (16.00), 1.081 (7.54), 1.165 (0.49), 1.232 (0.49), 1.352 (1.03), 1.434 (0.96), 1.519 (0.75), 1.529 (1.15), 1.542 (1.71), 1.551 (2.27), 1.560 (1.76), 1.572 (1.25), 1.582 (0.78), 1.605 (0.45), 1.637 (0.55), 1.660 (0.78), 1.666 (0.77), 1.677 (0.81), 1.683 (0.82), 1.689 (0.90), 1.697 (0.68), 1.707 (0.74), 1.713 (0.75), 1.729 (0.52), 1.738 (0.44), 1.917 (0.92), 1.926 (1.45), 1.931 (1.06), 1.950 (1.67), 1.957 (1.23), 1.967 (0.62), 2.053 (0.42), 2.062 (0.62), 2.078 (0.62), 2.083 (0.67), 2.087 (0.83), 2.092 (0.78), 2.109 (0.72), 2.114 (0.69), 2.116 (0.76), 2.139 (0.44), 2.518 (1.59), 2.523 (1.13), 2.687 (13.23), 2.926 (2.20), 2.945 (7.05), 2.963 (6.34), 2.981 (1.73), 3.625 (0.88), 3.639 (0.66), 3.643 (0.62), 3.653 (1.15), 3.659 (0.94), 3.667 (0.92), 3.674 (1.00), 3.688 (1.09), 3.700 (2.56), 3.925 (0.92), 3.953 (0.70), 3.956 (0.70), 5.492 (1.34), 5.498 (1.56), 5.518 (1.51), 5.523 (1.30), 6.732 (5.30), 6.739 (5.38), 8.011 (5.24), 8.017 (5.23).

### Intermediate 578

### N-methoxy-N-methyl-1-(oxan-2-yl)-1H-pyrazole-3-carboxamide

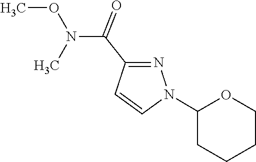 [SMILES: CON(C)C(=O)c1ccn(C2CCCCO2)n1]

N-Methoxy-N-methyl-1H-pyrazole-3-carboxamide (6.17 g, 67% purity, 26.6 mmol) was dissolved in 3,4-dihydro-2H-pyran (15 mL, 160 mmol). Trifluoro acetic acid (100 μL, 1.3 mmol) was added and the mixture was stirred for 5 h at 95° C. The reaction mixture was allowed to cool down and diluted with THF. Sodium bicarbonate solution and ethyl acetate were added the organic phase was washed twice with sodium bicarbonate solution, water and brine, dried over a hydrophobic filter and concentrated. The crude product was purified by column chromatography (silica gel, dichloromethane/methanol (0-7%) gradient) followed by a second column chromatography (silica gel, hexane/ethyl acetate (0-100%) gradient) to give 1.76 g (28% yield) of the title compound.

LC-MS (Method 1): Rₜ=0.80 min; MS (ESIpos): m/z=240 [M+H]⁺

¹H-NMR (400 MHz, DMSO-d6) δ[ppm]: 1.173 (0.66), 1.532 (0.49), 1.542 (0.68), 1.551 (0.60), 1.560 (0.52), 1.921 (0.49), 1.945 (0.53), 1.988 (1.15), 2.518 (0.48), 2.687 (16.00), 3.700 (13.11), 3.720 (0.78), 5.457 (0.45), 5.463 (0.53), 5.482 (0.52), 5.487 (0.44), 6.660 (1.81), 6.667 (1.76), 7.957 (1.69), 7.964 (1.73).

### Intermediate 579

### di-tert-butyl [5-bromo-3-(trifluoromethyl)pyrazin-2-yl]-2-imidodicarbonate

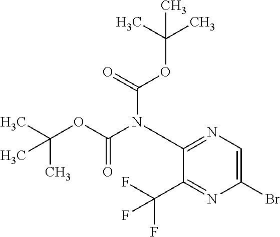 [SMILES: CC(C)(C)OC(=O)N(C(=O)OC(C)(C)C)c1ncc(Br)nc1C(F)(F)F]

5-Bromo-3-(trifluoromethyl)pyrazin-2-amine (5.00 g, 20.7 mmol) and di-tert-butyl dicarbonate (12 mL, 52 mmol) were dissolved in dichloromethane (150 mL). Triethylamine (2.9 mL, 21 mmol) and 4-dimethylaminopyridine (50.5 mg, 413 μmol) were added and the mixture was stirred 4 h at room temperature. The reaction mixture was concentrated. The residue was diluted with hexane. The precipitate was filtered off, washed and dried to give a first batch with 4.9 g (100% purity, 54% yield) of the title compound.

The filtrate was purified by column chromatography (silica gel, cyclohexane/ethyl acetate (0-20%) gradient) to give a second batch with 4.23 g (90% purity, 42% yield) of the title compound.

LC-MS (Method 5): Rₜ=2.42 min; MS (ESIpos): m/z=464 [M+Na]+⁺

¹H-NMR (400 MHz, DMSO-d6) δ[ppm]: 1.346 (16.00), 9.293 (0.80)

### Intermediate 580

### cyclopropyl(1H-1,2,3-triazol-5-yl)methanone

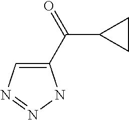

Sodium azide (212 mg, 3.27 mmol) was suspended in DMF (5 mL) and heated to 60° C. A solution of 1-cyclopropylprop-2-yn-1-one (500 mg, 41% purity, 2.18 mmol) in DMF (1.5 mL) was added dropwise and stirred for 1 h at 60° C. The reaction mixture was allowed to cool down, diluted with water and extracted three times with dichloromethane. The aqueous phase was acidified with 2 N hydrochloric acid and extracted twice with ethyl acetate. The organic phase was washed with brine, dried over a hydrophobic filter and concentrated to give 289 mg (96% yield) of the title compound.

LC-MS (Method 6): Rₜ=0.75 min; MS (ESIneg): m/z=136 [M−H]⁻

¹H NMR (DMSO-de, 400 MHz): δ (ppm) 15.75 (br s, 1H), 8.54 (br s, 1H), 2.99 (br s, 1H), 1.03-1.10 (m, 4H).

### Intermediate 581

### 1-cyclopropylprop-2-yn-1-one

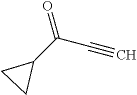

Cyclopropanecarbonyl chloride (1.00 g, 9.57 mmol) was dissolved in dichloromethane (7.2 mL). Ethynyl(trimethyl)silane (1.03 g, 10.5 mmol) was added, cooled to 0° C. and aluminum trichloride (3.83 g, 28.7 mmol) was added and stirred for 30 min at 0° C. and for 30 min at room temperature. The reaction mixture was diluted with 2 N hydrochloric acid and extracted with dichloromethane. The organic phase was washed with saturated sodium bicarbonate solution and brine, dried over a hydrophobic filter and concentrated to give 2.17 g (241% yield) of the title compound which was used without further purification in the next step.

### Intermediate 582

### 5-bromo-3-[(1R)-1-phenylethoxy]pyrazin-2-amine

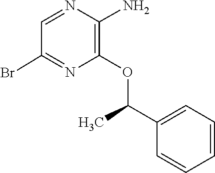 [SMILES: C[C@@H](Oc1nc(Br)cnc1N)c1ccccc1]

Sodium hydride (23.7 mg, 60% purity, 593 μmol) was dissolved in THF (1 mL) and cooled to 0° C. (1R)-1-Phenylethan-1-ol (72.5 mg, 593 μmol) was added slowly and stirred for 30 min at 0° C. A solution of 3,5-dibromopyrazin-2-amine (100 mg, 395 μmol) in THF was added and the mixture was stirred over night at reflux. The reaction mixture was allowed to cool down, diluted with water and extracted with ethyl acetate. The combined organic layers were washed with brine, dried over sodium sulfate, filtered and concentrated. The crude product was purified by column chromatography (silica gel, hexane/ethyl acetate gradient) followed by preparative HPLC to give 54.0 mg (46% yield) of the title compound.

LC-MS (Method 1): Rₜ=1.26 min; MS (ESIpos): m/z=294 [M+H]⁺

¹H-NMR (500 MHz, DMSO-d6) δ[ppm]: 1.582 (15.72), 1.595 (15.97), 2.699 (1.56), 3.301 (0.50), 3.315 (0.40), 5.756 (4.40), 6.056 (0.99), 6.069 (3.32), 6.082 (3.36), 6.095 (0.95), 6.593 (5.28), 7.265 (0.71), 7.268 (1.36), 7.270 (0.80), 7.278 (0.98), 7.282 (3.88), 7.287 (1.13), 7.294 (1.58), 7.297 (2.83), 7.299 (1.51), 7.343 (4.73), 7.358 (8.08), 7.369 (1.31), 7.372 (3.82), 7.492 (6.63), 7.507 (5.84), 7.509 (4.05), 7.552 (16.00).

### EXPERIMENTAL SECTION—EXAMPLES

### Example 1

### (rac)-2′-[6-amino-5-(trifluoromethyl)pyridin-3-yl]-N-[1-(2-chlorophenyl)cyclobutyl]-5′,6′-dihydrospiro[pyrrolidine-3,4′-pyrrolo[1,2-b]pyrazole]-1-carboxamide

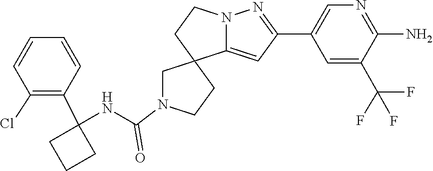 [SMILES: Nc1ncc(-c2cc3n(n2)CCC32CCN(C(=O)NC3(c4ccccc4Cl)CCC3)C2)cc1C(F)(F)F]

1-(2-Chlorophenyl)cyclobutan-1-amine-hydrochloride (50.0 mg, 229 μmol), N,N′-carbonyldiimidazole (40.6 mg, 250 μmol) and N,N-diisopropylethylamine (360 μL, 2.1 mmol) were solubilised in DMF (3.0 mL) and the mixture was stirred for 1 h at rt. (Rac)-5-[5′,6′-dihydrospiro[pyrrolidine-3,4′-pyrrolo[1,2-b]pyrazol]-2′-yl]-3-(trifluoromethyl)pyridin-2-amine-hydrochloride (75.0 mg, 208 μmol) solubilised in DMF was added dropwise and the mixture was stirred overnight at 60° C. The mixture was purified by preparative HPLC to give 5.90 mg (99% purity, 5% yield) of the target compound.

1H-NMR (400 MHz, DMSO-d6) δ[ppm]: 1.649 (0.74), 1.659 (1.29), 1.670 (1.83), 1.682 (1.83), 1.686 (1.89), 1.697 (2.04), 1.708 (1.54), 1.719 (0.94), 1.730 (0.52), 2.004 (2.70), 2.017 (4.47), 2.030 (5.91), 2.047 (4.86), 2.069 (2.58), 2.074 (2.81), 2.095 (1.61), 2.116 (0.45), 2.322 (0.67), 2.326 (0.95), 2.332 (0.64), 2.434 (0.42), 2.452 (1.89), 2.467 (4.34), 2.476 (6.63), 2.518 (6.13), 2.522 (5.23), 2.539 (2.60), 2.548 (3.18), 2.569 (1.41), 2.597 (1.96), 2.608 (2.31), 2.621 (3.72), 2.632 (3.25), 2.639 (2.61), 2.644 (2.93), 2.655 (2.90), 2.664 (1.86), 2.668 (2.43), 2.678 (1.42), 3.355 (2.66), 3.365 (3.07), 3.382 (7.91), 3.393 (9.26), 3.420 (2.19), 3.451 (1.46), 3.469 (2.70), 3.484 (1.96), 3.493 (1.76), 3.509 (0.87), 4.143 (5.61), 4.161 (11.19), 4.178 (5.36), 6.344 (12.15), 6.520 (10.84), 6.554 (16.00), 7.155 (2.53), 7.159 (2.66), 7.173 (6.08), 7.178 (6.23), 7.192 (5.65), 7.197 (5.41), 7.224 (4.29), 7.228 (5.50), 7.243 (6.22), 7.247 (7.62), 7.261 (3.30), 7.265 (3.30), 7.273 (9.75), 7.276 (8.03), 7.291 (6.50), 7.295 (5.85), 7.528 (6.30), 7.532 (6.33), 7.547 (5.70), 7.551 (5.41), 7.973 (8.88), 7.978 (8.93), 8.544 (8.28), 8.548 (8.09).

### Example 2

### (rac)-2-[6-amino-5-(trifluoromethyl)pyridin-3-yl]-N-[2-(2-fluorophenyl)propan-2-yl]-6,7-dihydrospiro[pyrazolo[5,1-c][1,4]oxazine-4,3′-pyrrolidine]-1′-carboxamide

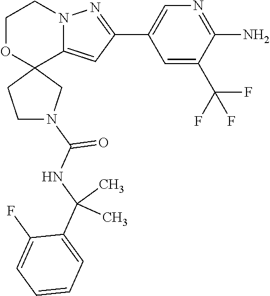 [SMILES: CC(C)(NC(=O)N1CCC2(C1)OCCn1nc(-c3cnc(N)c(C(F)(F)F)c3)cc12)c1ccccc1F]

Example 2 was prepared in analogy to Example 1 using (rac)-5-[6,7-dihydrospiro[pyrazolo[5,1-c][1,4]oxazine-4,3′-pyrrolidin]-2-yl]-3-(trifluoromethyl)pyridin-2-amine-hydrochloride and 2-(2-fluorophenyl)propan-2-amine-hydrochloride.

1H-NMR (400 MHz, DMSO-d6) δ[ppm]: 1.066 (0.63), 1.629 (15.95), 1.635 (16.00), 2.212 (0.78), 2.221 (0.87), 2.247 (1.23), 2.270 (0.66), 2.315 (1.04), 2.327 (1.43), 2.346 (0.72), 2.362 (0.61), 2.522 (1.29), 3.363 (0.72), 3.389 (1.23), 3.406 (1.24), 3.431 (0.57), 3.471 (1.84), 3.501 (2.15), 3.570 (0.94), 3.591 (1.66), 3.613 (0.75), 3.779 (1.95), 3.808 (1.66), 4.069 (0.59), 4.084 (1.25), 4.101 (1.78), 4.119 (2.96), 4.130 (5.04), 4.142 (5.33), 6.114 (5.49), 6.600 (6.89), 6.760 (10.59), 7.030 (1.27), 7.033 (1.42), 7.050 (1.64), 7.053 (1.85), 7.062 (1.32), 7.065 (1.58), 7.074 (1.64), 7.078 (1.68), 7.084 (2.16), 7.093 (3.41), 7.096 (2.79), 7.112 (2.30), 7.115 (1.95), 7.190 (0.85), 7.194 (1.02), 7.202 (1.01), 7.207 (1.42), 7.214 (1.35), 7.221 (1.32), 7.226 (1.32), 7.233 (0.70), 7.240 (0.61), 7.245 (0.55), 7.300 (1.39), 7.303 (1.38), 7.321 (2.22), 7.341 (1.22), 7.345 (1.07), 8.014 (4.23), 8.019 (4.31), 8.583 (4.00), 8.587 (3.98).

### Example 3

### (rac)-2-[6-amino-5-(trifluoromethyl)pyridin-3-yl]-N-(1-phenylcyclobutyl)-6,7-dihydrospiro[pyrazolo[5,1-c][1,4]oxazine-4,3′-pyrrolidine]-1′-carboxamide

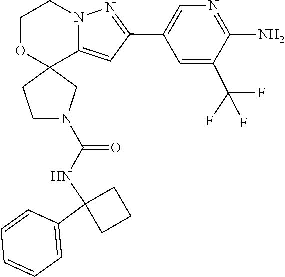 [SMILES: Nc1ncc(-c2cc3n(n2)CCOC32CCN(C(=O)NC3(c4ccccc4)CCC3)C2)cc1C(F)(F)F]

Example 3 was prepared in analogy to Example 1 using (rac)-5-[6,7-dihydrospiro[pyrazolo[5,1-c][1,4]oxazine-4,3′-pyrrolidin]-2-yl]-3-(trifluoromethyl)pyridin-2-amine and 1-phenylcyclobutan-1-amine-hydrochloride.

1H-NMR (400 MHz, DMSO-d6) δ[ppm]: 0.999 (0.75), 1.016 (0.86), 1.042 (0.43), 1.742 (0.89), 1.759 (1.68), 1.764 (1.79), 1.780 (1.79), 1.786 (2.00), 1.803 (1.25), 1.825 (0.50), 1.964 (0.86), 1.970 (1.07), 1.986 (1.90), 2.009 (1.75), 2.013 (1.68), 2.030 (1.04), 2.036 (1.00), 2.052 (0.64), 2.074 (0.64), 2.176 (0.61), 2.207 (1.36), 2.233 (1.90), 2.257 (1.00), 2.318 (2.04), 2.322 (2.86), 2.326 (3.94), 2.331 (3.15), 2.351 (1.72), 2.368 (2.29), 2.374 (2.43), 2.398 (3.65), 2.419 (2.90), 2.441 (3.54), 2.464 (4.94), 2.518 (8.88), 2.522 (5.69), 2.664 (1.54), 2.669 (2.08), 2.673 (1.54), 3.379 (2.47), 3.449 (2.40), 3.478 (2.72), 3.558 (1.61), 3.580 (2.76), 3.603 (1.29), 3.757 (3.29), 3.785 (2.83), 4.051 (1.11), 4.066 (2.04), 4.081 (2.79), 4.099 (3.22), 4.111 (3.54), 4.119 (5.87), 4.133 (10.49), 4.184 (0.79), 6.418 (1.22), 6.555 (0.97), 6.599 (12.78), 6.702 (8.20), 6.748 (16.00), 7.138 (1.54), 7.141 (2.76), 7.144 (1.83), 7.159 (6.80), 7.164 (2.68), 7.174 (2.68), 7.178 (4.65), 7.181 (2.58), 7.256 (0.61), 7.270 (7.80), 7.290 (12.42), 7.303 (2.54), 7.309 (7.19), 7.413 (10.99), 7.416 (12.74), 7.433 (9.91), 7.437 (8.09), 8.004 (7.59), 8.009 (7.55), 8.572 (7.55), 8.576 (7.41).

### Example 4

### (rac)-2′-[6-amino-5-(trifluoromethyl)pyridin-3-yl]-N-[2-(4-fluorophenyl)propan-2-yl]-5′,6′-dihydrospiro[pyrrolidine-3,4′-pyrrolo[1,2-b]pyrazole]-1-carboxamide

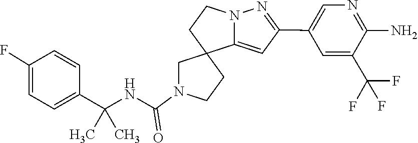 [SMILES: CC(C)(NC(=O)N1CCC2(CCn3nc(-c4cnc(N)c(C(F)(F)F)c4)cc32)C1)c1ccc(F)cc1]

Example 4 was prepared in analogy to Example 1 using (rac)-5-[5′,6′-dihydrospiro[pyrrolidine-3,4′-pyrrolo[1,2-b]pyrazol]-2′-yl]-3-(trifluoromethyl)pyridin-2-amine-hydrochloride and 2-(4-fluorophenyl)propan-2-amine.

1H-NMR (400 MHz, DMSO-d6) δ[ppm]: 1.547 (15.16), 1.556 (16.00), 2.024 (0.40), 2.038 (1.09), 2.052 (2.16), 2.069 (2.59), 2.073 (3.02), 2.086 (1.07), 2.099 (0.53), 2.518 (3.81), 2.534 (3.73), 2.539 (3.86), 2.553 (2.37), 3.394 (0.53), 3.413 (1.09), 3.420 (1.32), 3.449 (5.63), 3.476 (0.57), 3.532 (0.59), 3.549 (1.12), 3.564 (0.86), 3.575 (0.82), 3.590 (0.43), 4.175 (2.65), 4.193 (4.91), 4.210 (2.63), 6.071 (5.18), 6.456 (11.78), 6.558 (6.90), 7.020 (3.48), 7.025 (1.21), 7.042 (7.50), 7.059 (1.29), 7.064 (3.99), 7.072 (0.49), 7.351 (3.60), 7.357 (1.63), 7.365 (4.02), 7.374 (3.80), 7.382 (1.47), 7.388 (3.30), 8.008 (3.90), 8.013 (3.95), 8.148 (0.63), 8.591 (3.68), 8.596 (3.63).

### Example 5

### (rac)-2′-[6-amino-5-(trifluoromethyl)pyridin-3-yl]-N-[2-(2-chlorophenyl)propan-2-yl]-5′,6′-dihydrospiro[pyrrolidine-3,4′-pyrrolo[1,2-b]pyrazole]-1-carboxamide

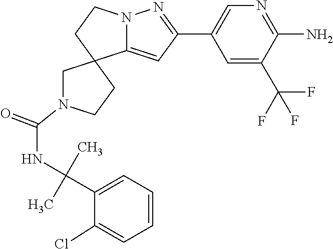 [SMILES: CC(C)(NC(=O)N1CCC2(CCn3nc(-c4cnc(N)c(C(F)(F)F)c4)cc32)C1)c1ccccc1Cl]

Example 5 was prepared in analogy to Example 1 using (rac)-5-[5′,6′-dihydrospiro[pyrrolidine-3,4′-pyrrolo[1,2-b]pyrazol]-2′-yl]-3-(trifluoromethyl)pyridin-2-amine-hydrochloride and 2-(2-chlorophenyl)propan-2-amine-hydrochloride.

¹H-NMR (400 MHz, DMSO-d6) δ[ppm]: 1.231 (0.74), 1.683 (16.00), 1.692 (15.08), 2.033 (1.15), 2.047 (2.21), 2.064 (2.62), 2.081 (1.09), 2.094 (0.51), 2.336 (0.60), 2.518 (11.25), 2.522 (6.20), 2.529 (2.70), 2.678 (0.65), 3.382 (0.71), 3.416 (8.52), 3.443 (0.79), 3.488 (0.68), 3.506 (1.27), 3.521 (0.92), 3.530 (0.82), 3.546 (0.42), 4.162 (2.58), 4.180 (5.48), 4.198 (2.51), 5.759 (8.09), 6.121 (5.58), 6.432 (11.74), 6.559 (7.21), 7.157 (1.11), 7.161 (1.12), 7.176 (2.60), 7.180 (2.73), 7.195 (2.50), 7.199 (2.38), 7.227 (1.68), 7.231 (2.24), 7.247 (2.35), 7.250 (2.99), 7.265 (1.51), 7.269 (1.51), 7.275 (4.51), 7.279 (3.76), 7.294 (3.07), 7.298 (2.67), 7.457 (2.72), 7.461 (2.81), 7.477 (2.35), 7.481 (2.26), 7.995 (4.07), 8.000 (4.11), 8.204 (0.64), 8.575 (3.81), 8.579 (3.77).

### Example 6

### (rac)-2-[6-amino-5-(trifluoromethyl)pyridin-3-yl]-N-[(1R)-1-(2-chloro-4-cyanophenyl)ethyl]-6,7-dihydrospiro[pyrazolo[5,1-c][1,4]oxazine-4,3′-pyrrolidine]-1′-carboxamide

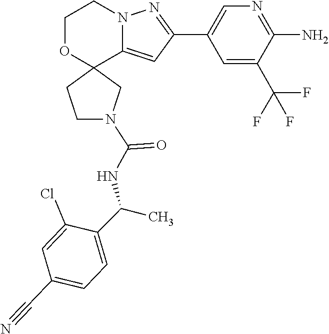 [SMILES: C[C@@H](NC(=O)N1CCC2(C1)OCCn1nc(-c3cnc(N)c(C(F)(F)F)c3)cc12)c1ccc(C#N)cc1Cl]

Example 6 was prepared in analogy to Example 1 using (rac)-tert-butyl-2-[6-amino-5-(trifluoromethyl)pyridin-3-yl]-6,7-dihydrospiro[pyrazolo[5,1-c][1,4]oxazine-4,3′-pyrrolidine]-1′-carboxylate and 4-[(1R)-1-aminoethyl]-3-chlorobenzonitrile.

1H-NMR (400 MHz, DMSO-d6) δ[ppm]: 0.829 (0.56), 1.052 (0.68), 1.329 (15.85), 1.347 (16.00), 2.073 (5.58), 2.202 (0.49), 2.226 (1.48), 2.254 (1.78), 2.278 (0.82), 2.322 (0.51), 2.327 (0.74), 2.332 (0.85), 2.354 (1.81), 2.518 (1.97), 2.522 (1.27), 2.669 (0.51), 3.387 (1.42), 3.404 (1.60), 3.430 (1.68), 3.449 (2.11), 3.478 (1.55), 3.514 (1.26), 3.544 (1.53), 3.559 (1.05), 3.583 (1.54), 3.606 (0.68), 3.644 (0.88), 3.666 (1.57), 3.689 (0.72), 3.773 (1.93), 3.802 (1.60), 3.850 (1.98), 3.877 (1.71), 4.052 (0.76), 4.066 (1.61), 4.082 (2.54), 4.100 (2.81), 4.112 (3.46), 4.123 (4.58), 4.132 (6.17), 4.138 (8.08), 4.144 (9.33), 5.108 (0.48), 5.120 (1.82), 5.126 (2.02), 5.138 (2.90), 5.144 (3.04), 5.156 (1.95), 5.162 (1.92), 5.172 (0.49), 6.600 (12.74), 6.760 (8.29), 6.766 (9.95), 6.784 (2.39), 6.803 (2.28), 6.822 (2.24), 6.841 (2.09), 7.661 (4.45), 7.681 (7.00), 7.700 (5.28), 7.819 (3.39), 7.823 (3.51), 7.832 (3.83), 7.836 (4.19), 7.839 (3.00), 7.843 (2.97), 7.851 (2.84), 7.856 (3.00), 7.988 (10.66), 7.991 (10.30), 8.008 (4.39), 8.014 (6.71), 8.021 (4.68), 8.136 (3.18), 8.576 (4.07), 8.581 (4.19), 8.587 (4.49), 8.592 (4.20).

### Example 7

### (rac)-2′-[6-amino-5-(trifluoromethyl)pyridin-3-yl]-N-[(1R)-1-(2-chloro-4-cyanophenyl)ethyl]-5′,6′-dihydrospiro[pyrrolidine-3,4′-pyrrolo[1,2-b]pyrazole]-1-carboxamide

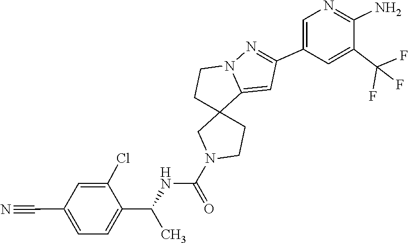 [SMILES: C[C@@H](NC(=O)N1CCC2(CCn3nc(-c4cnc(N)c(C(F)(F)F)c4)cc32)C1)c1ccc(C#N)cc1Cl]

Example 7 was prepared in analogy to Example 1 using (rac)-5-[5′,6′-dihydrospiro[pyrrolidine-3,4′-pyrrolo[1,2-b]pyrazol]-2′-yl]-3-(trifluoromethyl)pyridin-2-amine-hydrochloride and 4-[(1R)-1-aminoethyl]-3-chlorobenzonitrile.

1H-NMR (400 MHz, DMSO-d6) δ[ppm]: 1.323 (9.01), 1.330 (9.90), 1.341 (9.38), 1.347 (9.52), 2.074 (3.85), 2.083 (3.94), 2.518 (4.04), 2.523 (5.54), 2.539 (15.01), 2.558 (3.80), 3.382 (0.47), 3.429 (2.86), 3.453 (5.44), 3.483 (3.00), 3.493 (3.75), 3.518 (2.25), 3.530 (1.50), 3.546 (0.94), 3.556 (0.75), 3.574 (0.70), 3.594 (0.94), 4.172 (3.05), 4.177 (3.28), 4.190 (5.35), 4.194 (5.96), 4.207 (3.19), 4.212 (2.82), 5.111 (0.56), 5.128 (2.53), 5.146 (3.94), 5.164 (2.53), 5.181 (0.56), 6.472 (16.00), 6.544 (12.57), 6.773 (2.21), 6.789 (3.66), 6.807 (2.16), 7.642 (3.05), 7.663 (3.94), 7.705 (2.96), 7.725 (4.08), 7.775 (2.39), 7.778 (2.39), 7.795 (1.83), 7.799 (1.92), 7.808 (3.24), 7.812 (3.28), 7.828 (2.11), 7.832 (2.25), 7.980 (6.71), 7.984 (8.73), 7.989 (6.10), 8.014 (6.57), 8.133 (0.70), 8.590 (6.57), 8.595 (6.43).

### Example 8

(rac)-2′-[6-amino-5-(trifluoromethyl)pyridin-3-yl]-N-[(1S)-1-(3-chloropyridin-4-yl)ethyl]-5′,6′-dihydrospiro[pyrrolidine-3,4′-pyrrolo[1,2-b]pyrazole]-1-carboxamide

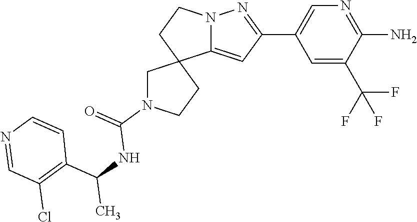 [SMILES: C[C@H](NC(=O)N1CCC2(CCn3nc(-c4cnc(N)c(C(F)(F)F)c4)cc32)C1)c1ccncc1Cl]

Example 8 was prepared in analogy to Example 1 using (rac)-5-[5′,6′-dihydrospiro[pyrrolidine-3,4′-pyrrolo[1,2-b]pyrazol]-2′-yl]-3-(trifluoromethyl)pyridin-2-amine-hydrochloride and (1S)-1-(3-chloropyridin-4-yl)ethan-1-amine-hydrochloride.

¹H-NMR (400 MHz, DMSO-d6) δ[ppm]: 1.232 (0.89), 1.332 (12.11), 1.336 (12.56), 1.350 (12.44), 1.353 (12.48), 1.902 (3.12), 2.085 (4.66), 2.323 (1.66), 2.327 (2.35), 2.332 (1.70), 2.518 (10.21), 2.523 (6.97), 2.530 (5.51), 2.548 (7.90), 2.566 (4.82), 2.660 (0.81), 2.665 (1.74), 2.669 (2.43), 2.673 (1.74), 3.420 (0.81), 3.438 (3.40), 3.464 (6.64), 3.491 (4.62), 3.520 (2.55), 3.539 (1.78), 3.555 (1.09), 3.565 (0.89), 3.583 (0.81), 3.603 (1.13), 3.627 (0.85), 4.179 (4.17), 4.195 (7.37), 4.211 (3.93), 5.036 (0.57), 5.054 (2.35), 5.072 (3.61), 5.088 (2.39), 5.106 (0.61), 6.469 (16.00), 6.552 (13.73), 6.762 (2.63), 6.779 (3.93), 6.795 (2.43), 7.454 (3.48), 7.466 (3.56), 7.532 (3.77), 7.544 (3.85), 8.014 (7.53), 8.019 (7.74), 8.375 (0.41), 8.435 (4.09), 8.448 (3.89), 8.477 (5.87), 8.490 (5.63), 8.525 (11.50), 8.528 (11.42), 8.588 (7.53), 8.593 (7.45).

### Example 9

### (rac)-2′-[6-amino-5-(trifluoromethyl)pyridin-3-yl]-N-[2-(3-fluorophenyl)propan-2-yl]-5′,6′-dihydrospiro[pyrrolidine-3,4′-pyrrolo[1,2-b]pyrazole]-1-carboxamide

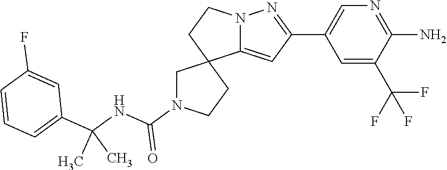 [SMILES: CC(C)(NC(=O)N1CCC2(CCn3nc(-c4cnc(N)c(C(F)(F)F)c4)cc32)C1)c1cccc(F)c1]

Example 9 was prepared in analogy to Example 1 using (rac)-5-[5′,6′-dihydrospiro[pyrrolidine-3,4′-pyrrolo[1,2-b]pyrazol]-2′-yl]-3-(trifluoromethyl)pyridin-2-amine-hydrochloride and 2-(3-fluorophenyl)propan-2-amine.

1H-NMR (400 MHz, DMSO-d6) δ[ppm]: 1.550 (14.22), 1.556 (16.00), 2.047 (1.03), 2.062 (1.97), 2.076 (2.35), 2.092 (1.00), 2.327 (0.78), 2.523 (4.68), 2.544 (3.82), 2.561 (2.43), 2.669 (0.78), 3.426 (1.55), 3.451 (3.61), 3.466 (3.18), 3.492 (0.78), 3.542 (0.54), 3.559 (1.01), 4.178 (2.35), 4.196 (4.13), 4.213 (2.35), 6.121 (4.44), 6.444 (8.64), 6.556 (6.03), 6.925 (0.79), 6.931 (0.92), 6.946 (1.50), 6.951 (1.89), 6.967 (0.88), 6.974 (0.93), 7.112 (1.64), 7.117 (1.33), 7.140 (1.63), 7.175 (1.73), 7.196 (2.52), 7.257 (1.22), 7.276 (2.11), 7.292 (1.90), 7.312 (0.76), 8.003 (3.38), 8.009 (3.60), 8.584 (3.25), 8.588 (3.36).

### Example 10

### (rac)-2-[6-amino-5-(trifluoromethyl)pyridin-3-yl]-N-[1-(pyridin-4-yl)cyclobutyl]-6,7-dihydrospiro[pyrazolo[5,1-c][1,4]oxazine-4,3′-pyrrolidine]-1′-carboxamide

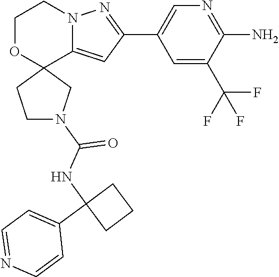 [SMILES: Nc1ncc(-c2cc3n(n2)CCOC32CCN(C(=O)NC3(c4ccncc4)CCC3)C2)cc1C(F)(F)F]

Example 10 was prepared in analogy to Example 1 using (rac)-5-[6,7-dihydrospiro[pyrazolo[5,1-c][1,4]oxazine-4,3′-pyrrolidin]-2-yl]-3-(trifluoromethyl)pyridin-2-amine-hydrochloride and 1-(pyridin-4-yl)cyclobutan-1-amine.

¹H-NMR (400 MHz, DMSO-d6) δ[ppm]: 1.009 (0.86), 1.026 (0.86), 1.231 (1.35), 1.822 (2.02), 1.843 (2.40), 1.861 (1.59), 2.007 (2.35), 2.030 (2.16), 2.074 (6.87), 2.221 (1.87), 2.245 (2.40), 2.269 (1.30), 2.327 (5.33), 2.344 (3.84), 2.387 (5.24), 2.411 (7.78), 2.430 (7.35), 2.451 (5.24), 2.669 (2.88), 3.471 (2.45), 3.498 (2.74), 3.602 (3.17), 3.770 (3.75), 3.797 (3.36), 4.062 (1.44), 4.078 (2.93), 4.093 (3.56), 4.129 (10.38), 4.141 (14.32), 6.604 (16.00), 6.765 (14.75), 6.884 (8.84), 7.039 (0.82), 7.386 (15.23), 7.390 (10.19), 7.402 (14.75), 7.638 (0.48), 8.010 (9.61), 8.014 (9.90), 8.140 (14.94), 8.205 (0.58), 8.476 (15.57), 8.480 (10.09), 8.491 (14.32), 8.577 (8.98).

### Example 11

### (rac)-2′-[6-amino-5-(trifluoromethyl)pyridin-3-yl]-N-[2-(3-chloropyridin-4-yl)propan-2-yl]-5′,6′-dihydrospiro[pyrrolidine-3,4′-pyrrolo[1,2-b]pyrazole]-1-carboxamide

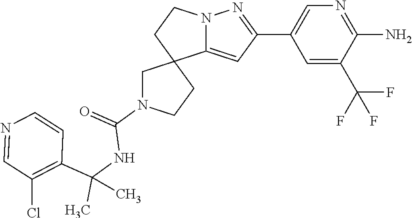 [SMILES: CC(C)(NC(=O)N1CCC2(CCn3nc(-c4cnc(N)c(C(F)(F)F)c4)cc32)C1)c1ccncc1Cl]

Example 11 was prepared in analogy to Example 1 using (rac)-5-[5′,6′-dihydrospiro[pyrrolidine-3,4′-pyrrolo[1,2-b]pyrazol]-2′-yl]-3-(trifluoromethyl)pyridin-2-amine-hydrochloride and 2-(3-chloropyridin-4-yl)propan-2-amine.

¹H-NMR (400 MHz, DMSO-d6) δ[ppm]: 1.164 (0.49), 1.232 (0.79), 1.247 (0.70), 1.257 (0.82), 1.332 (0.78), 1.560 (0.62), 1.654 (16.00), 2.036 (0.59), 2.051 (1.09), 2.067 (1.30), 2.084 (0.56), 2.322 (0.47), 2.327 (0.65), 2.332 (0.48), 2.518 (3.72), 2.522 (2.95), 2.537 (0.96), 2.664 (0.46), 2.669 (0.66), 2.673 (0.48), 2.728 (2.37), 2.888 (3.01), 3.417 (4.16), 3.440 (0.54), 3.505 (0.64), 3.521 (0.48), 3.528 (0.43), 4.165 (1.43), 4.183 (3.04), 4.200 (1.41), 5.758 (1.02), 6.332 (2.86), 6.443 (5.96), 6.558 (3.76), 7.427 (2.41), 7.440 (2.44), 8.001 (2.08), 8.006 (2.16), 8.147 (3.29), 8.389 (3.42), 8.402 (3.34), 8.408 (6.45), 8.578 (1.96), 8.581 (1.98).

### Example 12

### (rac)-2′-[6-amino-5-(trifluoromethyl)pyridin-3-yl]-N-(1-phenylcyclobutyl)-5′,6′-dihydrospiro[pyrrolidine-3,4′-pyrrolo[1,2-b]pyrazole]-1-carboxamide

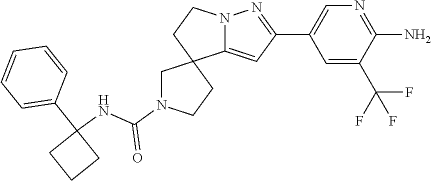 [SMILES: Nc1ncc(-c2cc3n(n2)CCC32CCN(C(=O)NC3(c4ccccc4)CCC3)C2)cc1C(F)(F)F]

Example 12 was prepared in analogy to Example 1 using (rac)-5-[5′,6′-dihydrospiro[pyrrolidine-3,4′-pyrrolo[1,2-b]pyrazol]-2′-yl]-3-(trifluoromethyl)pyridin-2-amine-hydrochloride and 1-phenylcyclobutan-1-amine-hydrochloride.

1H-NMR (400 MHz, DMSO-d6) δ[ppm]: 0.999 (0.66), 1.015 (0.69), 1.042 (0.42), 1.056 (1.13), 1.066 (0.98), 1.072 (1.13), 1.153 (3.41), 1.171 (6.70), 1.189 (3.35), 1.230 (2.11), 1.243 (2.86), 1.248 (1.82), 1.259 (3.21), 1.262 (3.13), 1.279 (2.49), 1.745 (0.76), 1.762 (1.46), 1.768 (1.56), 1.784 (1.62), 1.790 (1.80), 1.807 (1.14), 1.829 (0.44), 1.957 (0.79), 1.963 (0.94), 1.979 (1.86), 1.986 (13.20), 2.002 (1.85), 2.021 (1.54), 2.033 (2.34), 2.048 (3.61), 2.061 (4.05), 2.078 (1.98), 2.326 (0.65), 2.354 (1.36), 2.376 (2.75), 2.385 (3.45), 2.401 (4.11), 2.407 (3.48), 2.422 (2.50), 2.447 (2.01), 2.470 (3.84), 2.526 (7.40), 2.530 (6.83), 2.546 (4.36), 2.668 (0.61), 3.127 (0.43), 3.383 (0.98), 3.401 (3.15), 3.426 (8.82), 3.436 (6.98), 3.462 (1.72), 3.509 (1.57), 3.526 (2.71), 3.542 (2.34), 3.552 (2.35), 3.569 (1.92), 3.662 (3.47), 3.999 (1.02), 4.016 (2.82), 4.034 (2.80), 4.052 (1.00), 4.141 (0.47), 4.167 (4.66), 4.184 (8.94), 4.201 (4.69), 6.424 (16.00), 6.597 (1.82), 6.702 (7.56), 6.745 (0.79), 7.011 (0.86), 7.127 (2.25), 7.146 (5.62), 7.164 (3.82), 7.255 (6.46), 7.275 (10.79), 7.294 (6.17), 7.419 (9.98), 7.437 (8.42), 7.440 (6.54), 7.700 (0.55), 8.009 (6.99), 8.014 (7.10), 8.574 (6.54), 8.579 (6.46).

### Example 13

### (rac)-2-[6-amino-5-(trifluoromethyl)pyridin-3-yl]-N-[2-(pyridin-2-yl)propan-2-yl]-6,7-dihydrospiro[pyrazolo[5,1-c][1,4]oxazine-4,3′-pyrrolidine]-1′-carboxamide

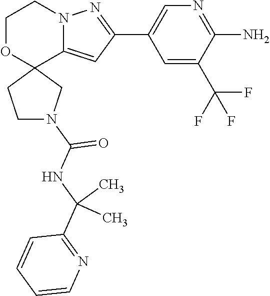 [SMILES: CC(C)(NC(=O)N1CCC2(C1)OCCn1nc(-c3cnc(N)c(C(F)(F)F)c3)cc12)c1ccccn1]

Example 13 was prepared in analogy to Example 1 using (rac)-5-[6,7-dihydrospiro[pyrazolo[5,1-c][1,4]oxazine-4,3′-pyrrolidin]-2-yl]-3-(trifluoromethyl)pyridin-2-amine-hydrochloride and 2-(pyridin-2-yl)propan-2-amine.

1H-NMR (400 MHz, DMSO-d6) δ[ppm]: 1.177 (0.43), 1.194 (0.45), 1.232 (0.58), 1.579 (16.00), 1.585 (15.80), 2.075 (1.24), 2.240 (0.63), 2.265 (0.86), 2.290 (0.45), 2.332 (1.59), 2.337 (1.21), 2.352 (0.88), 2.368 (0.48), 2.385 (0.43), 2.518 (5.38), 2.523 (3.58), 2.540 (0.66), 2.674 (1.09), 2.679 (0.48), 3.387 (0.68), 3.411 (0.96), 3.429 (0.91), 3.454 (0.45), 3.501 (1.19), 3.530 (1.41), 3.605 (0.73), 3.626 (1.31), 3.648 (0.61), 3.801 (1.59), 3.829 (1.34), 4.097 (0.86), 4.115 (1.56), 4.136 (5.15), 4.146 (3.89), 6.425 (4.06), 6.601 (5.70), 6.776 (9.44), 7.171 (1.62), 7.174 (1.72), 7.184 (1.64), 7.186 (1.89), 7.190 (1.89), 7.193 (1.74), 7.202 (1.84), 7.205 (1.84), 7.438 (1.74), 7.440 (3.08), 7.458 (2.07), 7.461 (3.33), 7.698 (1.79), 7.702 (1.74), 7.717 (2.07), 7.721 (2.12), 7.736 (1.41), 7.740 (1.31), 8.017 (3.41), 8.022 (3.46), 8.163 (2.22), 8.449 (1.84), 8.451 (2.15), 8.453 (2.25), 8.455 (1.92), 8.461 (1.92), 8.463 (2.22), 8.465 (2.07), 8.467 (1.77), 8.587 (3.18), 8.591 (3.18).

### Example 14

### (rac)-2′-(5-acetyl-6-aminopyridin-3-yl)-N-(1-phenylcyclobutyl)-5′,6′-dihydrospiro[pyrrolidine-3,4′-pyrrolo[1,2-b]pyrazole]-1-carboxamide

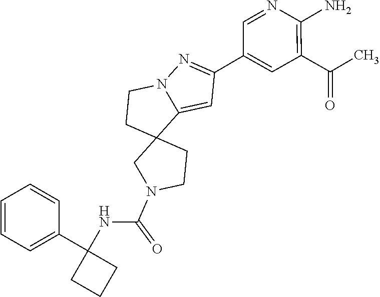 [SMILES: CC(=O)c1cc(-c2cc3n(n2)CCC32CCN(C(=O)NC3(c4ccccc4)CCC3)C2)cnc1N]

(Rac)-1-[(1-phenylcyclobutyl)carbamoyl]-5′,6′-dihydrospiro[pyrrolidine-3,4′-pyrrolo[1,2-b]pyrazol]-2′-yl trifluoromethanesulfonate (194 mg, 400 μmol), (5-acetyl-6-aminopyridin-3-yl)boronic acid (82.8 mg, 460 μmol), XPhos Pd G2 (47.2 mg, 60.0 μmol) and potassium phosphate (2.4 mL, 0.50 M, 1.2 mmol) were solubilised in dioxane (4.0 mL) and the mixture was stirred for 1 h at 100° C. The mixture was diluted with water and extracted three times with ethyl acetate. The combined organic layers were dried and evaporated. The residue was purified by preparative HPLC to give 27.5 mg (97% purity, 14% yield) of the target compound.

1H-NMR (400 MHz, DMSO-d6) δ[ppm]: 1.036 (1.47), 1.053 (2.85), 1.071 (1.56), 1.764 (0.45), 1.769 (0.47), 1.786 (0.49), 1.791 (0.56), 1.982 (0.53), 1.997 (0.41), 2.005 (0.50), 2.009 (0.51), 2.026 (0.41), 2.032 (0.44), 2.045 (0.71), 2.059 (1.09), 2.072 (1.24), 2.089 (0.56), 2.359 (0.40), 2.382 (0.86), 2.390 (1.00), 2.397 (0.83), 2.405 (1.22), 2.411 (0.97), 2.427 (0.71), 2.452 (0.70), 2.475 (1.30), 2.518 (2.01), 2.523 (1.96), 2.537 (1.90), 2.542 (1.85), 2.558 (1.18), 2.596 (16.00), 3.160 (3.62), 3.172 (3.72), 3.418 (0.85), 3.423 (1.03), 3.447 (3.80), 3.535 (0.65), 3.551 (0.49), 3.560 (0.45), 4.101 (0.67), 4.114 (0.66), 4.181 (1.46), 4.198 (2.81), 4.215 (1.43), 6.463 (4.93), 6.704 (2.38), 7.134 (0.71), 7.137 (0.43), 7.152 (1.77), 7.167 (0.72), 7.170 (1.19), 7.173 (0.69), 7.262 (1.98), 7.282 (3.33), 7.296 (0.73), 7.301 (1.92), 7.421 (2.89), 7.423 (3.12), 7.441 (2.60), 7.445 (2.01), 8.381 (2.64), 8.387 (2.78), 8.597

### Example 15

### (rac)-2-(6-amino-5-cyanopyridin-3-yl)-N-[1-(2-chlorophenyl)cyclobutyl]-6,7-dihydrospiro[pyrazolo[5,1-c][1,4]oxazine-4,3′-pyrrolidine]-1′-carboxamide

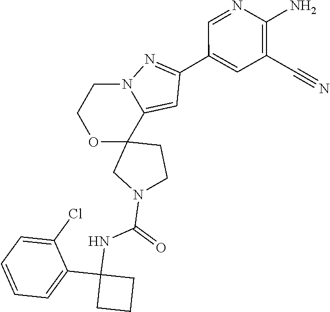 [SMILES: N#Cc1cc(-c2cc3n(n2)CCOC32CCN(C(=O)NC3(c4ccccc4Cl)CCC3)C2)cnc1N]

1-(2-Chlorophenyl)cyclobutan-1-amine hydrochloride (55.8 mg, 256 μmol), 1,1′-carbonyldiimidazole (41.5 mg, 256 μmol) and N,N-diisopropylethylamine (150 μL, 850 μmol) were stirred in DMF (1.0 mL) for 1 h at rt, then (rac)-2-amino-5-[6,7-dihydrospiro[pyrazolo[5,1-c][1,4]oxazine-4,3′-pyrrolidin]-2-yl]pyridine-3-carbonitrile hydrochloride (71.0 mg, 213 μmol) in DMF (1.0 mL) was added and stirred at rt overnight. Water was added to the mixture and purified by HPLC to afford 26.0 mg (95% purity, 23% yield) of the title compound.

¹H-NMR (400 MHz, DMSO-d6) δ[ppm]: 0.931 (2.90), 0.948 (2.92), 1.652 (0.65), 1.664 (1.10), 1.675 (1.60), 1.690 (1.67), 1.702 (1.78), 1.712 (1.33), 1.723 (0.77), 1.734 (0.43), 2.029 (1.32), 2.050 (2.36), 2.074 (6.68), 2.098 (1.44), 2.119 (0.53), 2.131 (0.65), 2.164 (1.40), 2.188 (1.86), 2.213 (0.94), 2.281 (1.65), 2.297 (1.94), 2.318 (1.68), 2.322 (2.22), 2.326 (2.88), 2.332 (2.43), 2.451 (0.99), 2.518 (8.72), 2.522 (6.66), 2.544 (2.14), 2.565 (0.94), 2.646 (3.19), 2.660 (2.48), 2.664 (3.05), 2.668 (3.22), 2.673 (2.43), 3.365 (0.98), 3.392 (2.48), 3.420 (2.83), 3.502 (1.48), 3.525 (2.65), 3.549 (1.19), 3.720 (2.76), 3.750 (2.36), 4.022 (1.15), 4.037 (1.94), 4.053 (2.92), 4.072 (2.92), 4.085 (3.48), 4.093 (4.09), 4.112 (9.21), 6.539 (9.98), 6.663 (16.00), 7.044 (14.11), 7.176 (2.05), 7.181 (2.20), 7.195 (5.77), 7.199 (5.53), 7.214 (5.23), 7.218 (5.24), 7.230 (4.31), 7.234 (4.92), 7.249 (5.67), 7.252 (6.78), 7.267 (2.85), 7.271 (2.46), 7.298 (8.25), 7.302 (7.98), 7.317 (5.47), 7.321 (5.39), 7.522 (5.56), 7.527 (5.44), 7.541 (5.20), 7.546 (4.68), 8.084 (10.87), 8.090 (11.42), 8.575 (12.43), 8.580 (12.81).

### Example 16

### (rac)-2′-(6-amino-5-cyanopyridin-3-yl)-N-[1-(2-chlorophenyl)cyclobutyl]-5′,6′-dihydrospiro[pyrrolidine-3,4′-pyrrolo[1,2-b]pyrazole]-1-carboxamide

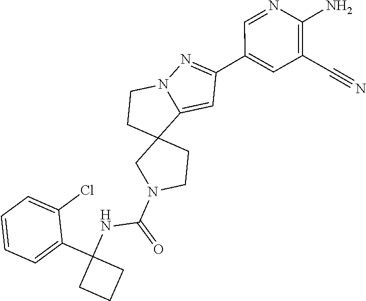 [SMILES: N#Cc1cc(-c2cc3n(n2)CCC32CCN(C(=O)NC3(c4ccccc4Cl)CCC3)C2)cnc1N]

1-(2-Chlorophenyl)cyclobutan-1-amine hydrochloride (63.6 mg, 292 μmol), 1,1′-carbonyldiimidazole (47.3 mg, 292 μmol) and N,N-diisopropylethylamine (170 μL, 970 μmol) were stirred in DMF (1 mL) for 1 h at rt, then (rac)-2-amino-5-[5′,6′-dihydrospiro[pyrrolidine-3,4′-pyrrolo[1,2-b]pyrazol]-2′-yl]pyridine-3-carbonitrile hydrochloride (77.0 mg, 243 μmol) in DMF (1.0 mL) was added and stirred at rt overnight. Water was added to the mixture and purified by HPLC to afford 50.0 mg (95% purity, 40% yield) of the title compound.

¹H-NMR (400 MHz, DMSO-d6) δ[ppm]: 0.932 (2.38), 0.948 (2.33), 1.648 (1.13), 1.670 (2.19), 1.687 (1.88), 1.697 (2.02), 1.708 (1.46), 1.719 (0.81), 2.002 (3.39), 2.020 (5.99), 2.047 (3.39), 2.068 (2.62), 2.073 (2.69), 2.094 (1.72), 2.117 (0.92), 2.124 (0.81), 2.131 (0.68), 2.146 (1.73), 2.157 (0.47), 2.174 (0.42), 2.199 (0.41), 2.322 (1.69), 2.326 (2.27), 2.332 (1.62), 2.386 (0.47), 2.407 (0.82), 2.413 (0.96), 2.435 (1.16), 2.454 (2.06), 2.466 (4.40), 2.518 (10.61), 2.522 (8.42), 2.548 (2.78), 2.570 (1.24), 2.600 (1.85), 2.611 (2.21), 2.623 (3.54), 2.634 (3.07), 2.646 (2.77), 2.659 (3.18), 2.664 (2.91), 2.669 (3.53), 2.673 (2.51), 2.678 (1.57), 2.720 (4.16), 3.289 (0.40), 3.378 (8.40), 3.387 (9.66), 3.411 (2.46), 3.438 (1.47), 3.456 (2.75), 3.471 (1.82), 3.480 (1.75), 3.496 (0.81), 4.141 (5.33), 4.159 (10.80), 4.176 (5.06), 6.310 (11.34), 6.524 (10.14), 7.000 (16.00), 7.168 (2.30), 7.173 (2.61), 7.187 (6.10), 7.192 (5.86), 7.206 (5.69), 7.210 (5.84), 7.223 (0.96), 7.230 (5.17), 7.233 (5.54), 7.248 (5.97), 7.252 (6.98), 7.267 (3.32), 7.271 (4.33), 7.275 (2.11), 7.288 (9.52), 7.291 (8.38), 7.307 (5.83), 7.310 (5.44), 7.341 (0.98), 7.362 (0.67), 7.530 (6.16), 7.535 (5.99), 7.549 (5.48), 7.554 (5.04), 8.074 (11.61), 8.079 (12.11), 8.571 (14.30), 8.576 (14.19).

### Example 17

### (rac)-2-[6-amino-5-(trifluoromethyl)pyridin-3-yl]-N-[2-(3-chloropyridin-4-yl)propan-2-yl]-6,7-dihydrospiro[pyrazolo[5,1-c][1,4]oxazine-4,3′-pyrrolidine]-1′-carboxamide

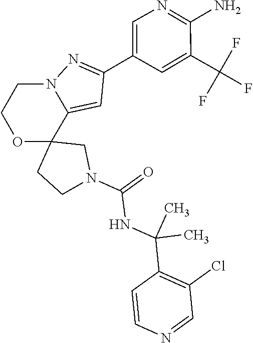 [SMILES: CC(C)(NC(=O)N1CCC2(C1)OCCn1nc(-c3cnc(N)c(C(F)(F)F)c3)cc12)c1ccncc1Cl]

To half of the reaction mixture of 3-chloro-4-(2-isocyanatopropan-2-yl)pyridine (80.0 mg, 407 μmol) was added N,N-diisopropylethylamine (180 μL, 1.0 mmol) and (rac)-5-[6,7-dihydrospiro[pyrazolo[5,1-c][1,4]oxazine-4,3′-pyrrolidin]-2-yl]-3-(trifluoromethyl)pyridin-2-amine hydrochloride (127 mg, 339 μmol). The mixture was stirred at rt overnight under nitrogen atmosphere and then 5 h at 60° C. Saturated potassium carbonate solution was added and stirred for 5 min. The layers were separated and the aqueous phase was extracted three times with dichloromethane (+10% methanol). The combined organic phases were concentrated to give 240 mg of a residue which was purified by flash chromatography to afford 82.0 mg (90% purity, 41% yield) of the title compound.

¹H NMR (DMSO-d₆) δ: 8.58 (d, 1H), 8.37-8.45 (m, 2H), 8.02 (d, 1H), 7.43 (d, 1H), 6.75 (s, 1H), 6.60 (s, 2H), 6.36 (s, 1H), 4.04-4.19 (m, 4H), 3.76 (br d, 1H), 3.56 (br m, 1H), 3.46 (br d, 1H), 3.38 (br d, 1H), 2.29-2.34 (m, 1H), 2.17-2.27 (m, 1H), 1.66 (d, 6H)

### Example 18

### (rac)-2-(6-amino-5-cyanopyridin-3-yl)-N-[1-(pyridin-4-yl)cyclobutyl]-6,7-dihydrospiro[pyrazolo[5,1-c][1,4]oxazine-4,3′-pyrrolidine]-1′-carboxamide

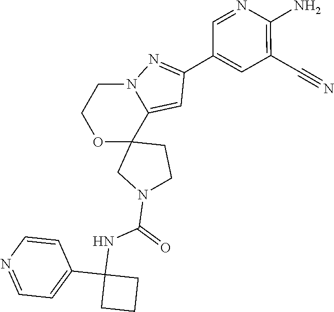 [SMILES: N#Cc1cc(-c2cc3n(n2)CCOC32CCN(C(=O)NC3(c4ccncc4)CCC3)C2)cnc1N]

1-(Pyridin-4-yl)cyclobutan-1-amine (37.9 mg, 256 μmol), 1,1′-carbonyldiimidazole (41.5 mg, 256 μmol) and N,N-diisopropylethylamine (150 μL, 850 μmol) was stirred in DMF (1.0 mL) for 1 h at rt, then (rac)-2-amino-5-[6,7-dihydrospiro[pyrazolo[5,1-c][1,4]oxazine-4,3′-pyrrolidin]-2-yl]pyridine-3-carbonitrile hydrochloride (71.0 mg, 213 μmol) in DMF (1.0 mL) was added and stirred at rt overnight. Water was added to the mixture and purified by HPLC to afford 37.0 mg (95% purity, 35% yield) of the title compound.

¹H-NMR (400 MHz, DMSO-d6) δ[ppm]: 1.821 (0.98), 1.843 (1.22), 1.861 (0.77), 2.006 (1.09), 2.029 (1.01), 2.194 (0.83), 2.220 (1.05), 2.323 (2.36), 2.327 (3.38), 2.332 (2.71), 2.387 (2.34), 2.411 (3.31), 2.430 (3.08), 2.452 (2.02), 2.518 (16.00), 2.523 (11.28), 2.665 (1.91), 2.669 (2.52), 2.673 (1.87), 3.295 (0.66), 3.304 (0.82), 3.385 (1.61), 3.445 (1.12), 3.476 (1.19), 3.602 (1.39), 3.768 (1.68), 3.797 (1.49), 4.060 (0.63), 4.075 (1.28), 4.092 (1.65), 4.125 (4.56), 4.136 (5.32), 6.714 (7.99), 6.890 (4.36), 7.051 (7.95), 7.386 (8.34), 7.390 (5.17), 7.398 (5.32), 7.402 (8.36), 8.115 (6.22), 8.121 (6.24), 8.476 (9.25), 8.481 (5.32), 8.488 (5.40), 8.491 (8.53), 8.597 (6.86), 8.603 (6.74).

### Example 19

### (rac)-2-[6-amino-5-(trifluoromethyl)pyridin-3-yl]-N-[1-(2-fluorophenyl)cyclobutyl]-6,7-dihydrospiro[pyrazolo[5,1-c][1,4]oxazine-4,3′-pyrrolidine]-1′-carboxamide

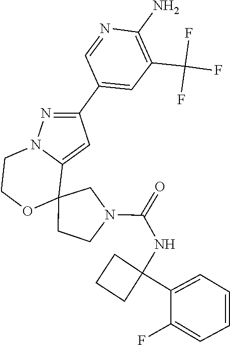 [SMILES: Nc1ncc(-c2cc3n(n2)CCOC32CCN(C(=O)NC3(c4ccccc4F)CCC3)C2)cc1C(F)(F)F]

1-(2-Fluorophenyl)cyclobutan-1-amine (52.8 mg, 319 μmol), 1,1′-carbonyldiimidazole (51.8 mg, 319 μmol) and N,N-diisopropylethylamine (280 μL, 1.6 mmol) were stirred in DMF (3 mL) for 1 h at rt, then (rac)-5-[6,7-dihydrospiro[pyrazolo[5,1-c][1,4]oxazine-4,3′-pyrrolidin]-2-yl]-3-(trifluoromethyl)pyridin-2-amine hydrochloride (100 mg, 266 μmol) in DMF (3 mL) was added and stirred at 60° C. overnight. Water was added to the mixture and purified by HPLC to afford 30.2 mg (95% purity, 20% yield) of the title compound.

¹H-NMR (400 MHz, DMSO-d6) δ[ppm]: 0.917 (0.45), 0.932 (3.79), 0.934 (1.29), 0.948 (3.92), 0.952 (0.66), 1.401 (1.31), 1.425 (1.11), 1.707 (0.51), 1.714 (0.62), 1.719 (0.76), 1.727 (1.17), 1.733 (1.19), 1.741 (1.29), 1.748 (1.09), 1.755 (1.37), 1.762 (0.80), 1.768 (0.86), 1.775 (0.60), 2.014 (0.90), 2.033 (1.54), 2.055 (1.42), 2.060 (1.40), 2.074 (4.20), 2.080 (0.88), 2.156 (0.45), 2.187 (1.05), 2.213 (1.44), 2.238 (0.76), 2.292 (1.31), 2.308 (1.60), 2.323 (1.68), 2.327 (2.01), 2.332 (1.46), 2.336 (1.05), 2.405 (0.45), 2.423 (0.45), 2.454 (0.78), 2.518 (7.24), 2.523 (6.95), 2.665 (0.80), 2.669 (1.13), 2.673 (0.82), 3.370 (0.86), 3.417 (2.05), 3.445 (2.40), 3.520 (1.27), 3.542 (2.30), 3.565 (1.03), 3.732 (2.58), 3.761 (2.20), 4.030 (0.90), 4.045 (1.44), 4.061 (2.22), 4.080 (2.15), 4.092 (2.71), 4.102 (2.99), 4.122 (7.51), 6.595 (16.00), 6.729 (11.84), 7.041 (1.81), 7.044 (1.99), 7.061 (2.40), 7.064 (2.71), 7.072 (5.03), 7.091 (8.70), 7.110 (3.32), 7.113 (2.60), 7.197 (1.21), 7.201 (1.37), 7.209 (1.40), 7.214 (2.13), 7.221 (1.83), 7.228 (1.74), 7.233 (1.99), 7.239 (0.96), 7.248 (0.86), 7.252 (0.80), 7.404 (1.85), 7.409 (1.87), 7.425 (3.04), 7.444 (1.74), 7.995 (5.76), 8.000 (5.93), 8.560 (5.46), 8.564 (5.39).

### Example 20

### (rac)-2′-(6-amino-5-cyanopyridin-3-yl)-N-(1-phenylcyclobutyl)-5′,6′-dihydrospiro[pyrrolidine-3,4′-pyrrolo[1,2-b]pyrazole]-1-carboxamide

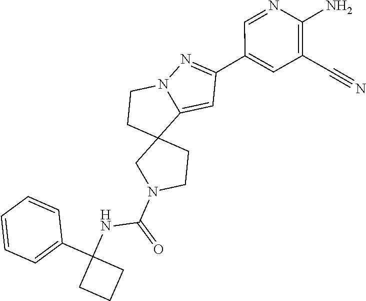 [SMILES: N#Cc1cc(-c2cc3n(n2)CCC32CCN(C(=O)NC3(c4ccccc4)CCC3)C2)cnc1N]

1-Phenylcyclobutan-1-amine hydrochloride (53.6 mg, 292 μmol), 1,1′-carbonyldiimidazole (47.3 mg, 292 μmol) and N,N-diisopropylethylamine (170 μL, 970 μmol) were stirred in DMF (1.0 mL) for 1 h at rt, then (rac)-2-amino-5-[5′,6′-dihydrospiro[pyrrolidine-3,4′-pyrrolo[1,2-b]pyrazol]-2′-yl]pyridine-3-carbonitrile hydrochloride (77.0 mg, 243 μmol) in DMF (1.0 mL) was added and stirred at rt overnight. Water was added to the mixture and purified by HPLC to afford 25.0 mg (98% purity, 22% yield) of the title compound.

¹H-NMR (400 MHz, DMSO-d6) δ[ppm]: 1.741 (0.74), 1.758 (1.41), 1.763 (1.52), 1.780 (1.55), 1.785 (1.74), 1.802 (1.06), 1.824 (0.42), 1.969 (0.89), 1.985 (1.66), 2.007 (1.53), 2.012 (1.46), 2.024 (0.83), 2.151 (0.58), 2.175 (1.27), 2.209 (1.69), 2.233 (0.87), 2.318 (2.18), 2.322 (3.46), 2.326 (4.74), 2.331 (3.60), 2.368 (1.92), 2.375 (1.89), 2.397 (2.95), 2.412 (2.43), 2.418 (2.57), 2.441 (3.19), 2.463 (4.84), 2.518 (12.23), 2.522 (7.48), 2.539 (0.56), 2.660 (0.96), 2.664 (2.19), 2.668 (2.95), 2.673 (2.19), 2.678 (0.96), 3.289 (0.44), 3.356 (1.76), 3.364 (1.83), 3.381 (1.70), 3.408 (0.92), 3.427 (2.11), 3.457 (2.40), 3.558 (1.35), 3.579 (2.43), 3.603 (1.10), 3.753 (3.06), 3.782 (2.60), 4.048 (1.03), 4.064 (1.94), 4.079 (2.31), 4.096 (3.09), 4.103 (3.16), 4.116 (6.34), 4.128 (9.60), 6.696 (16.00), 6.707 (7.85), 6.948 (0.42), 7.046 (12.25), 7.076 (0.60), 7.140 (1.43), 7.143 (2.44), 7.161 (5.83), 7.177 (2.32), 7.180 (4.03), 7.183 (2.23), 7.271 (6.82), 7.291 (10.85), 7.305 (2.21), 7.310 (6.44), 7.413 (9.83), 7.416 (10.71), 7.433 (8.48), 7.437 (6.30), 8.105 (10.31), 8.111 (10.94), 8.592 (12.03), 8.597 (11.75).

### Example 21

### (rac)-2-(6-amino-5-cyanopyridin-3-yl)-N-[1-(4-fluorophenyl)cyclobutyl]-6,7-dihydrospiro[pyrazolo[5,1-c][1,4]oxazine-4,3′-pyrrolidine]-1′-carboxamide

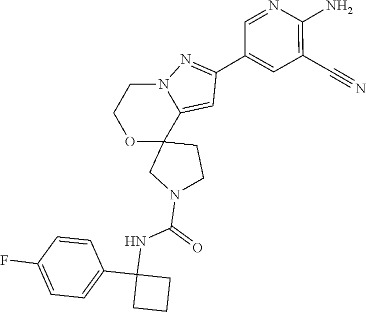 [SMILES: N#Cc1cc(-c2cc3n(n2)CCOC32CCN(C(=O)NC3(c4ccc(F)cc4)CCC3)C2)cnc1N]

1-(4-Fluorophenyl)cyclobutan-1-amine hydrochloride (51.6 mg, 256 μmol), 1,1′-carbonyldiimidazole (41.5 mg, 256 μmol) and N,N-diisopropylethylamine (150 μL, 850 μmol) were stirred in DMF (1.0 mL) for 1 h at rt, then (rac)-2-amino-5-[6,7-dihydrospiro[pyrazolo[5,1-c][1,4]oxazine-4,3′-pyrrolidin]-2-yl]pyridine-3-carbonitrile hydrochloride (71.0 mg, 213 μmol) in DMF (1.0 mL) was added and stirred at rt overnight. Water was added to the mixture and purified by HPLC to afford 52.0 mg (95% purity, 47% yield) of the title compound.

¹H-NMR (400 MHz, DMSO-d6) δ[ppm]: 1.252 (0.69), 1.723 (0.92), 1.739 (1.72), 1.745 (1.84), 1.761 (1.88), 1.767 (2.06), 1.784 (1.26), 1.805 (0.48), 1.960 (1.07), 1.976 (1.96), 1.998 (1.88), 2.026 (0.92), 2.042 (0.45), 2.074 (2.87), 2.144 (0.67), 2.177 (1.56), 2.203 (2.00), 2.227 (1.02), 2.326 (4.41), 2.331 (3.73), 2.361 (3.58), 2.370 (3.65), 2.386 (3.77), 2.402 (3.09), 2.418 (1.64), 2.432 (3.37), 2.453 (5.34), 2.522 (6.66), 2.665 (1.66), 2.669 (2.21), 2.673 (1.67), 3.354 (2.51), 3.372 (2.18), 3.397 (1.25), 3.418 (2.53), 3.447 (2.85), 3.550 (1.78), 3.572 (3.15), 3.596 (1.49), 3.748 (3.77), 3.777 (3.22), 4.045 (1.18), 4.060 (2.33), 4.076 (2.92), 4.093 (3.79), 4.113 (7.29), 4.126 (11.64), 6.701 (16.00), 6.723 (8.64), 7.046 (14.65), 7.078 (7.60), 7.083 (2.97), 7.100 (15.38), 7.117 (2.90), 7.122 (8.20), 7.422 (7.75), 7.427 (3.57), 7.436 (8.69), 7.444 (7.80), 7.452 (3.17), 7.458 (6.65), 8.108 (10.92), 8.114 (11.21), 8.592 (11.80), 8.598 (11.44).

### Example 22

### (rac)-2′-(6-amino-5-cyanopyridin-3-yl)-N-[1-(4-fluorophenyl)cyclobutyl]-5′,6′-dihydrospiro[pyrrolidine-3,4′-pyrrolo[1,2-b]pyrazole]-1-carboxamide

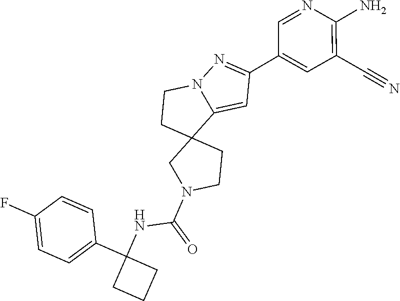 [SMILES: N#Cc1cc(-c2cc3n(n2)CCC32CCN(C(=O)NC3(c4ccc(F)cc4)CCC3)C2)cnc1N]

1-(4-Fluorophenyl)cyclobutan-1-amine hydrochloride (58.8 mg, 292 μmol), 1,1′-carbonyldiimidazole (47.3 mg, 292 μmol) and N,N-diisopropylethylamine (170 μL, 970 μmol) were stirred in DMF (1.0 mL) for 1 h at rt, then (rac)-2-amino-5-[5′,6′-dihydrospiro[pyrrolidine-3,4′-pyrrolo[1,2-b]pyrazol]-2′-yl]pyridine-3-carbonitrile hydrochloride (77.0 mg, 243 μmol) in DMF (1.0 mL) was added and stirred at rt overnight. Water was added to the mixture and purified by HPLC to afford 53.0 mg (99% purity, 46% yield) of the title compound.

¹H-NMR (400 MHz, DMSO-d6) δ[ppm]: 1.725 (0.63), 1.730 (0.56), 1.742 (1.22), 1.747 (1.30), 1.763 (1.31), 1.768 (1.48), 1.786 (0.91), 1.946 (0.62), 1.953 (0.80), 1.968 (1.43), 1.984 (1.10), 1.991 (1.35), 1.995 (1.39), 2.011 (1.15), 2.028 (2.29), 2.047 (3.72), 2.074 (0.74), 2.339 (1.28), 2.355 (1.58), 2.361 (2.12), 2.370 (2.82), 2.377 (2.09), 2.386 (3.28), 2.392 (2.61), 2.408 (1.94), 2.434 (1.24), 2.441 (1.56), 2.450 (2.13), 2.457 (2.85), 2.463 (2.93), 2.473 (3.18), 2.518 (7.89), 2.523 (6.69), 2.536 (2.73), 2.539 (2.87), 2.555 (0.56), 2.754 (0.90), 3.375 (0.83), 3.393 (1.86), 3.415 (11.30), 3.435 (1.18), 3.488 (0.92), 3.506 (1.81), 3.514 (1.18), 3.520 (1.27), 3.531 (1.23), 3.547 (0.59), 4.161 (3.96), 4.178 (7.82), 4.195 (3.80), 6.417 (16.00), 6.712 (6.60), 6.994 (11.00), 7.055 (0.67), 7.063 (5.90), 7.068 (1.95), 7.079 (2.56), 7.085 (12.56), 7.090 (2.24), 7.102 (2.08), 7.107 (6.63), 7.116 (0.64), 7.418 (0.77), 7.426 (6.04), 7.431 (2.51), 7.440 (6.62), 7.448 (6.08), 7.456 (2.26), 7.462 (5.28), 7.469 (0.50), 8.126 (9.78), 8.132 (9.36), 8.601 (10.42), 8.607 (10.75).

### Example 23

### (rac)-2′-[6-amino-5-(trifluoromethyl)pyridin-3-yl]-N-[1-(2-fluorophenyl)cyclobutyl]-5′,6′-dihydrospiro[pyrrolidine-3,4′-pyrrolo[1,2-b]pyrazole]-1-carboxamide

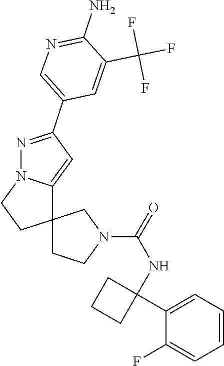 [SMILES: Nc1ncc(-c2cc3n(n2)CCC32CCN(C(=O)NC3(c4ccccc4F)CCC3)C2)cc1C(F)(F)F]

1-(2-Fluorophenyl)cyclobutan-1-amine (27.6 mg, 167 μmol), 1,1′-carbonyldiimidazole (27.0 mg, 167 μmol) and N,N-diisopropylethylamine (150 μL, 830 μmol) were stirred in DMF (1 mL) for 1 h at rt, then (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (50.0 mg, 139 μmol) in DMF (1.5 mL) was added and stirred at rt overnight. Water was added to the mixture and purified by HPLC to afford 6.00 mg (95% purity, 8% yield) of the title compound.

¹H-NMR (400 MHz, DMSO-d6) δ[ppm]: 0.949 (2.09), 0.965 (1.93), 1.704 (0.68), 1.712 (0.78), 1.717 (0.95), 1.725 (1.50), 1.731 (1.53), 1.739 (1.61), 1.745 (1.40), 1.753 (1.94), 1.766 (1.09), 1.774 (0.79), 1.787 (0.42), 2.009 (3.17), 2.026 (5.12), 2.036 (5.21), 2.049 (3.24), 2.054 (3.63), 2.068 (1.57), 2.084 (0.62), 2.094 (0.46), 2.323 (0.56), 2.327 (0.78), 2.332 (0.58), 2.457 (1.19), 2.518 (8.72), 2.524 (10.01), 2.547 (3.28), 2.576 (0.82), 2.665 (0.60), 2.669 (0.82), 2.673 (0.60), 3.368 (3.24), 3.397 (10.10), 3.403 (9.09), 3.429 (1.35), 3.470 (1.27), 3.488 (2.33), 3.496 (1.54), 3.502 (1.70), 3.514 (1.54), 3.529 (0.83), 4.151 (5.04), 4.169 (10.49), 4.186 (4.86), 6.389 (16.00), 6.549 (13.29), 6.582 (8.78), 7.025 (2.30), 7.027 (2.59), 7.045 (2.99), 7.048 (3.41), 7.054 (2.45), 7.057 (2.82), 7.064 (3.24), 7.067 (2.99), 7.077 (3.83), 7.083 (7.41), 7.086 (5.55), 7.102 (4.27), 7.105 (3.41), 7.182 (1.58), 7.186 (1.81), 7.195 (1.84), 7.200 (2.73), 7.206 (2.30), 7.214 (2.33), 7.219 (2.42), 7.225 (1.21), 7.233 (1.12), 7.238 (1.02), 7.407 (2.45), 7.412 (2.47), 7.428 (3.84), 7.431 (3.65), 7.448 (2.22), 7.452 (1.94), 7.984 (7.45), 7.989 (7.57), 8.556 (6.98), 8.561 (6.86).

### Example 24

### (rac)-2′-[6-amino-5-(trifluoromethyl)pyridin-3-yl]-N-[1-(3-fluorophenyl)cyclobutyl]-5′,6′-dihydrospiro[pyrrolidine-3,4′-pyrrolo[1,2-b]pyrazole]-1-carboxamide

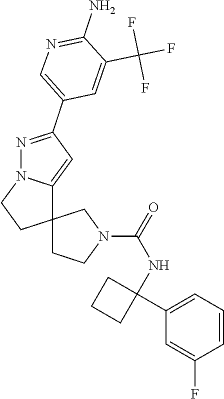 [SMILES: Nc1ncc(-c2cc3n(n2)CCC32CCN(C(=O)NC3(c4cccc(F)c4)CCC3)C2)cc1C(F)(F)F]

1-(3-Fluorophenyl)cyclobutan-1-amine hydrochloride (33.6 mg, 167 μmol), 1,1′-carbonyldiimidazole (27.0 mg, 167 μmol) and N,N-diisopropylethylamine (150 μL, 830 μmol) were stirred in DMF (1 mL) for 1 h at rt, then (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (50.0 mg, 139 μmol) in DMF (1 mL) was added and stirred 1.5 h at 60° C. Water was added to the mixture and purified by HPLC to afford 4.60 mg (96% purity, 6% yield) of the title compound.

¹H-NMR (400 MHz, DMSO-d6) δ[ppm]: 0.945 (2.14), 0.961 (2.04), 1.753 (0.63), 1.760 (0.62), 1.776 (1.24), 1.781 (1.28), 1.798 (1.32), 1.803 (1.53), 1.820 (0.93), 1.959 (0.60), 1.966 (0.77), 1.981 (1.45), 1.997 (1.07), 2.004 (1.38), 2.009 (1.43), 2.024 (1.18), 2.032 (1.18), 2.041 (1.77), 2.055 (2.85), 2.068 (3.17), 2.084 (1.64), 2.097 (0.59), 2.323 (0.58), 2.327 (0.81), 2.332 (0.60), 2.348 (0.98), 2.372 (2.15), 2.380 (2.68), 2.388 (2.09), 2.396 (3.24), 2.402 (2.73), 2.418 (2.12), 2.430 (1.87), 2.453 (3.13), 2.460 (2.26), 2.470 (2.50), 2.476 (2.49), 2.518 (5.46), 2.523 (3.29), 2.532 (5.37), 2.536 (5.37), 2.551 (3.64), 2.665 (0.60), 2.669 (0.84), 2.674 (0.58), 3.389 (0.88), 3.406 (2.63), 3.431 (6.90), 3.442 (5.07), 3.468 (1.18), 3.516 (0.88), 3.533 (1.69), 3.548 (1.22), 3.558 (1.21), 3.574 (0.60), 4.169 (4.12), 4.187 (7.49), 4.204 (3.99), 6.421 (16.00), 6.551 (10.69), 6.763 (7.02), 6.949 (1.18), 6.952 (1.28), 6.955 (1.49), 6.958 (1.47), 6.971 (2.56), 6.975 (2.46), 6.978 (2.78), 6.991 (1.39), 6.993 (1.49), 6.998 (1.62), 7.000 (1.55), 7.163 (2.04), 7.169 (2.63), 7.173 (2.23), 7.190 (2.12), 7.195 (2.67), 7.200 (2.16), 7.259 (2.58), 7.279 (5.25), 7.298 (3.05), 7.313 (3.16), 7.317 (3.96), 7.333 (3.82), 7.337 (1.62), 7.352 (1.45), 7.994 (6.07), 7.999 (6.17), 8.570 (5.65), 8.575 (5.62).

### Example 25

### (rac)-2′-(6-amino-5-cyanopyridin-3-yl)-N-[2-(3-chloropyridin-4-yl)propan-2-yl]-5′,6′-dihydrospiro[pyrrolidine-3,4′-pyrrolo[1,2-b]pyrazole]-1-carboxamide

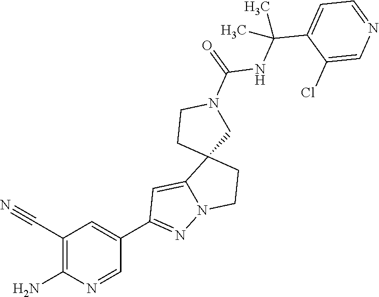 [SMILES: CC(C)(NC(=O)N1CC[C@]2(CCn3nc(-c4cnc(N)c(C#N)c4)cc32)C1)c1ccncc1Cl]

To half of the reaction mixture of 3-chloro-4-(2-isocyanatopropan-2-yl)pyridine (80.0 mg, 407 μmol) was added N,N-diisopropylethylamine (180 μL, 1.0 mmol) and (rac)-2-amino-5-[5′,6′-dihydrospiro[pyrrolidine-3,4′-pyrrolo[1,2-b]pyrazol]-2′-yl]pyridine-3-carbonitrile hydrochloride (107 mg, 339 μmol). The mixture was stirred at rt overnight under nitrogen atmosphere and then 5 h at 60° C. Saturated potassium carbonate solution was added and stirred for 5 min. The layers were separated and the aqueous phase was extracted three times with dichloromethane (+10% methanol). The combined organic phases were concentrated to give 190 mg of crude material which was purified by flash chromatography to afford 78.0 mg (90% purity, 43% yield) of the title compound.

1H-NMR (400 MHz, DMSO-d6) δ[ppm]: 1.231 (0.29), 1.653 (16.00), 2.056 (1.38), 2.074 (0.69), 2.457 (0.16), 2.518 (4.74), 2.522 (2.99), 2.534 (1.35), 2.549 (0.46), 2.567 (0.22), 3.381 (0.64), 3.407 (2.04), 3.421 (2.58), 3.446 (0.67), 3.478 (0.37), 3.495 (0.66), 3.511 (0.50), 3.520 (0.44), 3.537 (0.24), 4.162 (1.48), 4.180 (3.14), 4.198 (1.50), 6.331 (2.87), 6.431 (5.40), 7.003 (4.16), 7.426 (2.40), 7.439 (2.44), 8.127 (3.11), 8.132 (3.18), 8.390 (3.32), 8.403 (3.24), 8.419 (6.35), 8.600 (3.47), 8.606 (3.44).

### Example 26

### (rac)-2′-(6-amino-5-cyanopyridin-3-yl)-N-[1-(pyridin-4-yl)cyclobutyl]-5′,6′-dihydrospiro[pyrrolidine-3,4′-pyrrolo[1,2-b]pyrazole]-1-carboxamide

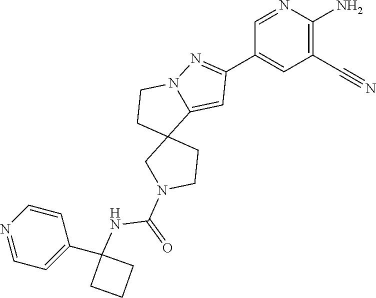 [SMILES: N#Cc1cc(-c2cc3n(n2)CCC32CCN(C(=O)NC3(c4ccncc4)CCC3)C2)cnc1N]

1-(Pyridin-4-yl)cyclobutan-1-amine (43.2 mg, 292 μmol), 1,1′-carbonyldiimidazole (47.3 mg, 292 μmol) and N,N-diisopropylethylamine (170 μL, 970 μmol) were stirred in DMF (1.0 mL) for 1 h at rt, then (rac)-2-amino-5-[5′,6′-dihydrospiro[pyrrolidine-3,4′-pyrrolo[1,2-b]pyrazol]-2′-yl]pyridine-3-carbonitrile hydrochloride (77.0 mg, 243 μmol) in DMF (1.0 mL) was added and stirred 3 h at 60° C. Water was added to the mixture and purified by HPLC to afford 39.0 mg (99% purity, 35% yield) of the title compound.

¹H-NMR (400 MHz, DMSO-d6) δ[ppm]: 1.803 (0.71), 1.826 (1.42), 1.849 (1.66), 1.867 (1.06), 1.889 (0.42), 2.001 (1.56), 2.015 (1.51), 2.028 (1.69), 2.051 (2.92), 2.067 (4.33), 2.074 (5.11), 2.347 (1.19), 2.377 (3.02), 2.393 (3.50), 2.415 (3.32), 2.434 (2.60), 2.452 (3.19), 2.518 (16.00), 2.523 (11.32), 2.535 (7.13), 2.555 (4.01), 3.440 (9.76), 3.534 (1.75), 4.170 (4.20), 4.188 (8.19), 4.205 (4.19), 6.440 (14.09), 6.884 (6.89), 7.002 (11.81), 7.411 (10.35), 7.415 (6.86), 7.422 (7.07), 7.426 (10.58), 8.138 (9.27), 8.144 (9.33), 8.472 (12.42), 8.475 (7.27), 8.483 (7.57), 8.487 (11.45), 8.608 (10.05), 8.614 (10.13).

### Example 27

### (rac)-2-(6-amino-5-cyanopyridin-3-yl)-N-(1-phenylcyclobutyl)-6,7-dihydrospiro[pyrazolo[5,1-c][1,4]oxazine-4,3′-pyrrolidine]-1′-carboxamide

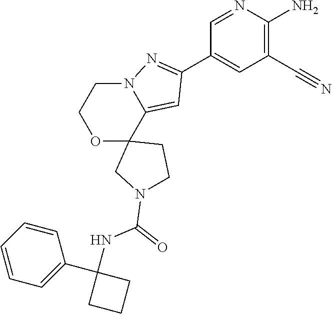 [SMILES: N#Cc1cc(-c2cc3n(n2)CCOC32CCN(C(=O)NC3(c4ccccc4)CCC3)C2)cnc1N]

1-Phenylcyclobutan-1-amine hydrochloride (47.0 mg, 256 μmol), 1,1′-carbonyldiimidazole (41.5 mg, 256 μmol) and N,N-diisopropylethylamine (150 μL, 850 μmol) were stirred in DMF (1.0 mL) for 1 h at rt, then (rac)-2-amino-5-[6,7-dihydrospiro[pyrazolo[5,1-c][1,4]oxazine-4,3′-pyrrolidin]-2-yl]pyridine-3-carbonitrile hydrochloride (71.0 mg, 213 μmol) in DMF (1.0 mL) was added and stirred overnight at rt. Water was added to the mixture and purified by HPLC to afford 26.0 mg (98% purity, 25% yield) of the title compound.

¹H-NMR (400 MHz, DMSO-d6) δ[ppm]: 1.760 (0.92), 1.765 (0.93), 1.782 (0.97), 1.788 (1.09), 1.805 (0.72), 1.978 (1.07), 2.000 (1.04), 2.028 (1.81), 2.050 (2.81), 2.064 (1.46), 2.336 (1.31), 2.356 (0.74), 2.385 (1.72), 2.401 (2.06), 2.423 (1.31), 2.518 (16.00), 2.522 (12.73), 2.543 (2.70), 2.678 (1.24), 2.762 (1.55), 3.290 (0.53), 3.348 (1.59), 3.370 (0.48), 3.422 (6.56), 3.494 (0.64), 3.510 (1.30), 3.535 (0.89), 4.162 (2.84), 4.180 (5.32), 4.197 (2.79), 6.392 (10.48), 6.697 (4.90), 6.999 (7.83), 7.138 (1.50), 7.156 (3.69), 7.171 (1.47), 7.174 (2.58), 7.178 (1.43), 7.263 (4.15), 7.283 (6.78), 7.301 (3.86), 7.418 (5.87), 7.421 (6.34), 7.439 (5.09), 7.442 (3.90), 8.111 (6.98), 8.117 (6.72), 8.596 (7.41), 8.602 (7.88).

### Example 28

### (rac)-2-(6-amino-5-cyanopyridin-3-yl)-N-[2-(4-fluorophenyl)propan-2-yl]-6,7-dihydrospiro[pyrazolo[5,1-c][1,4]oxazine-4,3′-pyrrolidine]-1′-carboxamide

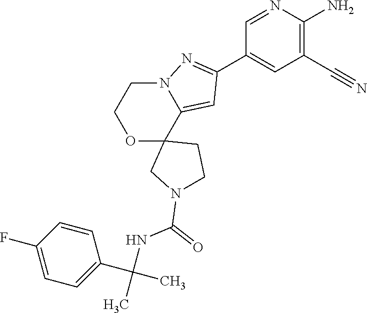 [SMILES: CC(C)(NC(=O)N1CCC2(C1)OCCn1nc(-c3cnc(N)c(C#N)c3)cc12)c1ccc(F)cc1]

2-(4-Fluorophenyl)propan-2-amine hydrochloride (48.6 mg, 256 μmol), 1,1′-carbonyldiimidazole (41.5 mg, 256 μmol) and N,N-diisopropylethylamine (150 μL, 850 μmol) were stirred in DMF (1.0 mL) for 1 h at rt, then (rac)-2-amino-5-[6,7-dihydrospiro[pyrazolo[5,1-c][1,4]oxazine-4,3′-pyrrolidin]-2-yl]pyridine-3-carbonitrile hydrochloride (71.0 mg, 213 μmol) in DMF (1.0 mL) was added and stirred overnight at rt. Water was added to the mixture and purified by HPLC to afford 35.0 mg (98% purity, 34% yield) of the title compound.

¹H-NMR (400 MHz, DMSO-d6) δ[ppm]: 1.549 (16.00), 1.555 (15.81), 2.074 (1.15), 2.178 (0.68), 2.186 (0.71), 2.212 (0.97), 2.236 (0.50), 2.311 (0.80), 2.322 (1.36), 2.326 (1.77), 2.331 (1.22), 2.336 (0.69), 2.342 (0.62), 2.359 (0.53), 2.518 (3.21), 2.522 (2.07), 2.664 (0.62), 2.668 (0.84), 2.673 (0.62), 3.361 (0.46), 3.385 (0.91), 3.403 (0.92), 3.429 (0.49), 3.449 (1.29), 3.478 (1.48), 3.578 (0.73), 3.599 (1.31), 3.622 (0.60), 3.781 (1.58), 3.809 (1.35), 4.068 (0.47), 4.082 (1.01), 4.099 (1.56), 4.124 (4.56), 4.134 (4.16), 4.150 (1.22), 6.102 (4.92), 6.714 (10.77), 7.046 (9.22), 7.063 (1.84), 7.069 (8.08), 7.074 (1.56), 7.086 (1.29), 7.091 (4.09), 7.099 (0.45), 7.343 (0.42), 7.351 (3.77), 7.357 (1.62), 7.365 (4.14), 7.373 (3.77), 7.382 (1.37), 7.387 (3.39), 8.118 (6.29), 8.124 (6.05), 8.602 (7.06), 8.607 (7.02).

### Example 29

### (rac)-2′-[6-amino-5-(trifluoromethyl)pyridin-3-yl]-N-[1-(pyridin-4-yl)cyclobutyl]-5′,6′-dihydrospiro[pyrrolidine-3,4′-pyrrolo[1,2-b]pyrazole]-1-carboxamide

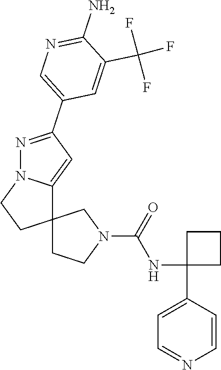 [SMILES: Nc1ncc(-c2cc3n(n2)CCC32CCN(C(=O)NC3(c4ccncc4)CCC3)C2)cc1C(F)(F)F]

1-(Pyridin-4-yl)cyclobutan-1-amine hydrochloride (36.9 mg, 167 μmol), 1,1′-carbonyldiimidazole (27.0 mg, 167 μmol) and N,N-diisopropylethylamine (150 μL, 830 μmol) were stirred in DMF (1.5 mL) for 1 h at rt, then (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (50.0 mg, 139 μmol) in DMF (1.5 mL) was added and stirred 1.5 h at 60° C. Water was added to the mixture and purified by HPLC to afford 19.4 mg (95% purity, 27% yield) of the title compound.

¹H-NMR (400 MHz, DMSO-d6) δ[ppm]: 0.840 (0.44), 0.858 (0.44), 0.919 (0.61), 0.934 (4.85), 0.950 (4.81), 1.237 (0.44), 1.803 (0.78), 1.825 (1.59), 1.848 (2.00), 1.866 (1.22), 1.888 (0.54), 2.000 (1.76), 2.023 (1.80), 2.050 (2.81), 2.065 (3.93), 2.078 (4.07), 2.323 (1.46), 2.327 (2.07), 2.332 (1.49), 2.344 (1.36), 2.375 (3.56), 2.391 (4.34), 2.413 (4.00), 2.444 (3.76), 2.518 (10.68), 2.523 (9.05), 2.537 (6.64), 2.543 (6.54), 2.558 (4.00), 2.665 (1.49), 2.669 (2.03), 2.673 (1.46), 3.445 (10.37), 3.549 (2.00), 4.173 (4.92), 4.190 (9.39), 4.208 (4.75), 6.458 (16.00), 6.556 (13.08), 6.872 (8.03), 7.390 (12.75), 7.395 (8.24), 7.402 (8.41), 7.406 (12.61), 8.009 (7.36), 8.014 (7.32), 8.458 (14.58), 8.461 (8.71), 8.469 (8.61), 8.473 (13.69), 8.584 (6.98), 8.588 (6.78).

### Example 30

### (rac)-2-[6-amino-5-(trifluoromethyl)pyridin-3-yl]-N-[1-(3-fluorophenyl)cyclobutyl]-6,7-dihydrospiro[pyrazolo[5,1-c][1,4]oxazine-4,3′-pyrrolidine]-1′-carboxamide

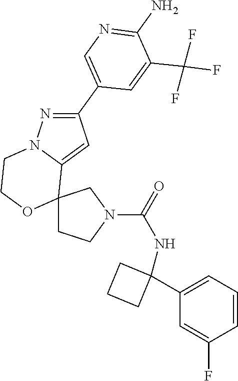 [SMILES: Nc1ncc(-c2cc3n(n2)CCOC32CCN(C(=O)NC3(c4cccc(F)c4)CCC3)C2)cc1C(F)(F)F]

1-(3-Fluorophenyl)cyclobutan-1-amine hydrochloride (64.4 mg, 319 μmol), 1,1′-carbonyldiimidazole (51.8 mg, 319 μmol) and N,N-diisopropylethylamine (280 μL, 1.6 mmol) were stirred in DMF (1 mL) for 1 h at rt, then (rac)-5-[6,7-dihydrospiro[pyrazolo[5,1-c][1,4]oxazine-4,3′-pyrrolidin]-2-yl]-3-(trifluoromethyl)pyridin-2-amine hydrochloride (100 mg, 266 μmol) in DMF (1 mL) was added and stirred overnight at rt. Water was added to the mixture and purified by HPLC to afford 73.6 mg (90% purity, 47% yield) of the title compound.

¹H-NMR (400 MHz, DMSO-d6) δ[ppm]: 0.917 (1.89), 0.931 (16.00), 0.934 (5.08), 0.948 (15.70), 0.952 (2.62), 1.004 (0.61), 1.021 (0.61), 1.401 (1.10), 1.425 (0.91), 1.753 (0.85), 1.759 (0.67), 1.770 (1.43), 1.776 (1.55), 1.787 (1.13), 1.793 (1.55), 1.798 (1.79), 1.809 (0.76), 1.815 (1.16), 1.836 (0.46), 1.966 (0.73), 1.971 (0.94), 1.988 (1.67), 1.993 (1.34), 2.004 (1.16), 2.010 (1.55), 2.015 (1.49), 2.026 (0.82), 2.031 (0.79), 2.038 (0.79), 2.182 (0.55), 2.214 (1.25), 2.240 (1.67), 2.264 (0.88), 2.322 (2.34), 2.327 (2.46), 2.332 (2.46), 2.336 (2.31), 2.347 (1.73), 2.358 (1.83), 2.364 (2.28), 2.370 (2.65), 2.380 (2.89), 2.387 (3.25), 2.396 (3.22), 2.404 (4.59), 2.410 (2.83), 2.422 (5.05), 2.440 (4.08), 2.446 (4.29), 2.463 (3.25), 2.471 (2.68), 2.478 (2.68), 2.518 (5.08), 2.523 (3.22), 2.665 (0.85), 2.669 (1.19), 2.673 (0.88), 2.940 (1.00), 2.957 (1.31), 2.973 (0.97), 3.370 (1.73), 3.389 (1.64), 3.413 (0.79), 3.456 (1.79), 3.485 (2.07), 3.562 (1.37), 3.584 (2.40), 3.608 (1.10), 3.762 (2.98), 3.791 (2.49), 4.056 (0.94), 4.070 (1.86), 4.086 (2.68), 4.112 (3.68), 4.121 (5.78), 4.135 (9.46), 6.598 (12.14), 6.753 (14.14), 6.769 (7.39), 6.965 (1.46), 6.967 (1.61), 6.971 (1.86), 6.974 (1.79), 6.987 (3.10), 6.993 (3.44), 7.007 (1.67), 7.009 (1.73), 7.014 (1.92), 7.015 (1.76), 7.164 (2.43), 7.169 (3.13), 7.174 (2.68), 7.191 (2.46), 7.195 (3.25), 7.201 (2.52), 7.253 (3.44), 7.273 (5.54), 7.311 (3.29), 7.326 (3.47), 7.331 (4.65), 7.347 (4.41), 7.350 (2.13), 7.366 (1.83), 8.004 (7.03), 8.009 (7.15), 8.543 (0.49), 8.572 (6.66), 8.576 (6.57).

### Example 31

### 2′-[6-amino-5-(trifluoromethyl)pyridin-3-yl]-N-{(1R)-1-[3-(trifluoromethyl)phenyl]ethyl}-5′,6′-dihydrospiro[pyrrolidine-3,4′-pyrrolo[1,2-b]pyrazole]-1-carboxamide (Mixture of Stereoisomers)

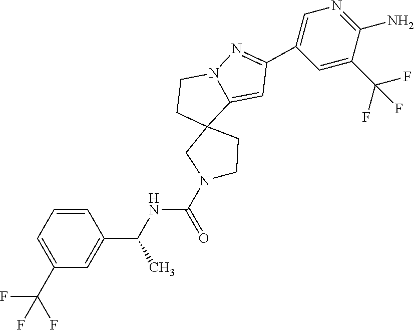 [SMILES: C[C@@H](NC(=O)N1CCC2(CCn3nc(-c4cnc(N)c(C(F)(F)F)c4)cc32)C1)c1cccc(C(F)(F)F)c1]

(1R)-1-[3-(trifluoromethyl)phenyl]ethan-1-amine (56.8 mg, 300 μmol), 1,1′-carbonyldiimidazole (48.7 mg, 300 μmol) and N,N-diisopropylethylamine (170 μL, 1.0 mmol) were stirred in DMF (5.0 mL) for 1 h at rt. (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (100 mg, 90% purity, 250 μmol) was added and the mixture was stirred for 2 h at 60° C. Water was added and the mixture was purified by preparative HPLC to give 38.0 mg (98% purity, 28% yield) of the title compound.

LC-MS (method 1): Rₜ=1.19 min; MS (ESIpos): m/z=539 [M+H]⁺

¹H-NMR (400 MHz, DMSO-d₆): δ [ppm]=1.39 (dd, 3H), 2.08 (br d, 2H), 2.52-2.57 (m, 2H), 3.41-3.49 (m, 3H), 3.50-3.62 (m, 1H), 4.19 (t, 2H), 4.92 (br t, 1H), 6.40-6.47 (m, 1H), 6.52-6.58 (m, 2H), 6.63 (dd, 1H), 7.49-7.59 (m, 2H), 7.61-7.76 (m, 2H), 8.00 (s, 1H), 8.56-8.60 (m, 1H).

### Example 32

### (rac)-2′-[6-amino-5-(trifluoromethyl)pyridin-3-yl]-N-[2-(3-fluoropyridin-2-yl)propan-2-yl]-5′,6′-dihydrospiro[pyrrolidine-3,4′-pyrrolo[1,2-b]pyrazole]-1-carboxamide

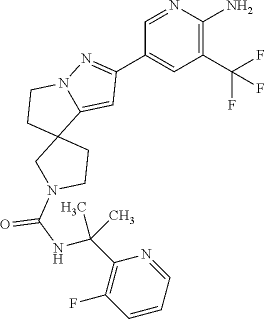 [SMILES: CC(C)(NC(=O)N1CCC2(CCn3nc(-c4cnc(N)c(C(F)(F)F)c4)cc32)C1)c1ncccc1F]

Example 32 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and 2-(3-fluoropyridin-2-yl)propan-2-amine (48.2 mg, 313 μmol).

LC-MS (method 1): Rₜ=1.09 min; MS (ESIpos): m/z=504 [M+H]⁺

¹H-NMR (400 MHz, DMSO-d6): δ [ppm]=1.63 (s, 6H), 2.01-2.14 (m, 2H), 2.52-2.57 (m, 2H), 3.38-3.47 (m, 3H), 3.49-3.58 (m, 1H), 4.19 (t, 2H), 6.45-6.50 (m, 2H), 6.55 (s, 2H), 7.33 (ddd, 1H), 7.55 (ddd, 1H), 8.01 (d, 1H), 8.31 (dt, 1H), 8.58 (d, 1H).

### Example 33

### (rac)-2′-[6-amino-5-(trifluoromethyl) pyridin-3-yl]-N-[2-(pyridin-4-yl)propan-2-yl]-5′,6′-dihydrospiro[pyrrolidine-3,4′-pyrrolo[1,2-b]pyrazole]-1-carboxamide

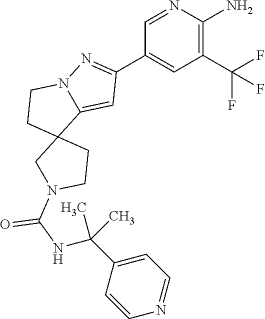 [SMILES: CC(C)(NC(=O)N1CCC2(CCn3nc(-c4cnc(N)c(C(F)(F)F)c4)cc32)C1)c1ccncc1]

Example 33 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and 2-(pyridin-4-yl)propan-2-amine (42.6 mg, 313 μmol).

LC-MS (method 1): Rₜ=0.96 min; MS (ESIpos): m/z=486 [M+H]⁺

¹H-NMR (400 MHz, DMSO-d6): δ [ppm]=1.53 (s, 3H), 1.54 (s, 3H), 2.00-2.14 (m, 2H), 2.52-2.58 (m, 2H), 3.39-3.50 (m, 3H), 3.53-3.62 (m, 1H), 4.20 (t, 2H), 6.22 (s, 1H), 6.47 (s, 1H), 6.56 (s, 2H), 7.30-7.33 (m, 2H), 8.02 (d, 1H), 8.40-8.44 (m, 2H), 8.60 (d, 1H).

### Example 34

### (rac)-2′-[6-amino-5-(trifluoromethyl)pyridin-3-yl]-N-[2-(2-fluorophenyl)propan-2-yl]-5′,6′-dihydrospiro[pyrrolidine-3,4′-pyrrolo[1,2-b]pyrazole]-1-carboxamide

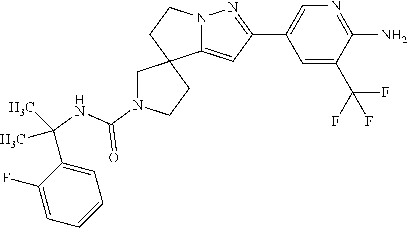 [SMILES: CC(C)(NC(=O)N1CCC2(CCn3nc(-c4cnc(N)c(C(F)(F)F)c4)cc32)C1)c1ccccc1F]

Example 34 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and 2-(2-fluorophenyl)propan-2-amine hydrochloride (43.5 mg, 229 μmol).

LC-MS (method 2): Rₜ=1.14 min; MS (ESIpos): m/z=504 [M+H]⁺

¹H-NMR (400 MHz, DMSO-d₆): δ [ppm]=1.63 (s, 6H), 1.98-2.16 (m, 2H), 2.52-2.55 (m, 2H), 3.44 (br d, 2H), 3.51-3.58 (m, 1H), 4.19 (t, 2H), 6.09 (s, 1H), 6.44 (s, 1H), 6.53-6.58 (m, 2H), 6.95-7.11 (m, 3H), 7.17-7.25 (m, 1H), 7.29-7.35 (m, 1H), 8.01 (d, 1H), 8.59 (d, 1H).

### Example 35

### 2′-[6-amino-5-(trifluoromethyl)pyridin-3-yl]-N-[(1R)-1-(3-fluorophenyl)ethyl]-5′,6′-dihydrospiro[pyrrolidine-3,4′-pyrrolo[1,2-b]pyrazole]-1-carboxamide (Mixture of Stereoisomers)

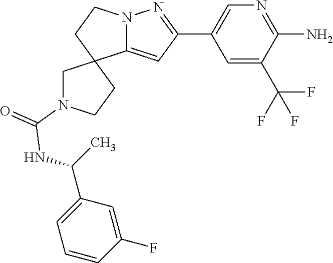 [SMILES: C[C@@H](NC(=O)N1CCC2(CCn3nc(-c4cnc(N)c(C(F)(F)F)c4)cc32)C1)c1cccc(F)c1]

Example 35 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and (1R)-1-(3-fluorophenyl)ethan-1-amine (31.9 mg, 229 μmol).

LC-MS (method 2): Rₜ=1.08 min; MS (ESIpos): m/z=489 [M+H]⁺

¹H NMR (400 MHz, DMSO-d₆) δ ppm 1.36 (dd, 3H) 2.02-2.12 (m, 2H) 2.52-2.60 (m, 2H) 3.42-3.50 (m, 3H) 3.51-3.63 (m, 1H) 4.19 (t, 2H) 4.85 (quin, 1H) 6.43-6.47 (m, 1H) 6.49-6.58 (m, 3H) 6.97-7.04 (m, 1H) 7.13-7.20 (m, 2H) 7.28-7.36 (m, 1H) 8.01 (d, 1H) 8.58 (s, 1H)

### Example 36

### (rac)-2′-[6-amino-5-(trifluoromethyl)pyridin-3-yl]-N-[2-(pyridin-3-yl)propan-2-yl]-5′,6′-dihydrospiro[pyrrolidine-3,4′-pyrrolo[1,2-b]pyrazole]-1-carboxamide

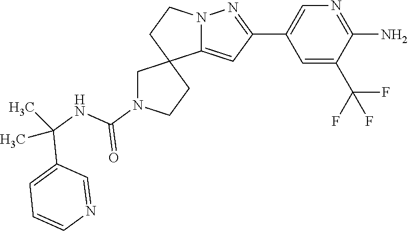 [SMILES: CC(C)(NC(=O)N1CCC2(CCn3nc(-c4cnc(N)c(C(F)(F)F)c4)cc32)C1)c1cccnc1]

Example 36 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and 2-(pyridin-3-yl)propan-2-amine (31.2 mg, 229 μmol).

LC-MS (Method 2): Rt=0.73 min; MS (ESIpos): m/z=486 [M+H]⁺

¹H NMR (400 MHz, DMSO-d₆) δ ppm 1.56-1.61 (m, 5H) 2.00-2.14 (m, 2H) 2.54 (t, 2H) 3.24-3.41 (m, 1H) 3.45-3.51 (m, 2H) 3.53-3.65 (m, 1H) 4.19 (t, 2H) 6.19 (s, 1H) 6.45 (s, 1H) 6.56 (s, 2H) 7.27 (dd, 1H) 7.68-7.74 (m, 1H) 8.03 (d, 1H) 8.15 (s, 1H) 8.35 (br d, 1H) 8.57-8.62 (m, 2H)

### Example 37

### (rac)-2′-[6-amino-5-(trifluoromethyl)pyridin-3-yl]-N-[1-(pyridin-3-yl)cyclobutyl]-5′,6′-dihydrospiro[pyrrolidine-3,4′-pyrrolo[1,2-b]pyrazole]-1-carboxamide

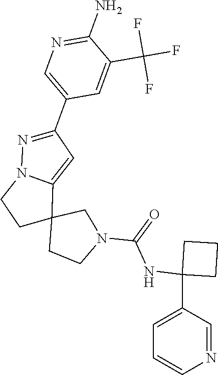 [SMILES: Nc1ncc(-c2cc3n(n2)CCC32CCN(C(=O)NC3(c4cccnc4)CCC3)C2)cc1C(F)(F)F]

Example 37 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (50.0 mg, 139 μmol) and 1-(pyridin-3-yl)cyclobutan-1-amine (24.7 mg, 167 μmol).

LC-MS (method 1): Rₜ=0.97 min; MS (ESIpos): m/z=499 [M+H]⁺

¹H NMR (400 MHz, DMSO-d₆) δ ppm 1.74-1.86 (m, 1H) 1.96-2.11 (m, 3H) 2.40-2.47 (m, 3H) 2.52-2.58 (m, 3H) 3.38-3.49 (m, 3H) 3.54 (dt, 1H) 4.18 (t, 2H) 6.43 (s, 1H) 6.55 (s, 2H) 6.83 (s, 1H) 7.28-7.33 (m, 1H) 7.80 (dt, 1H) 8.01 (d, 1H) 8.37 (dd, 1H) 8.58 (d, 1H) 8.64 (d, 1H)

### Example 38

### 2′-[6-amino-5-(trifluoromethyl)pyridin-3-yl]-N-[(1R)-1-(4-fluorophenyl)ethyl]-5′,6′-dihydrospiro[pyrrolidine-3,4′-pyrrolo[1,2-b]pyrazole]-1-carboxamide (Mixture of Stereoisomers)

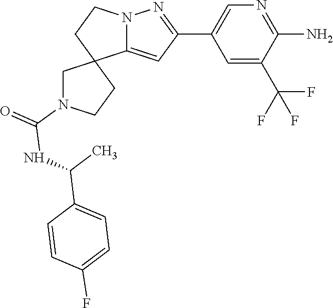 [SMILES: C[C@@H](NC(=O)N1CCC2(CCn3nc(-c4cnc(N)c(C(F)(F)F)c4)cc32)C1)c1ccc(F)cc1]

Example 38 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and (1R)-1-(4-fluorophenyl)ethan-1-amine (31.9 mg, 229 μmol).

LC-MS (method 2): Rₜ=1.08 min; MS (ESIpos): m/z=489 [M+H]⁺

¹H NMR (400 MHz, DMSO-d₆) δ ppm 1.35 (dd, 3H) 2.02-2.12 (m, 2H) 2.52-2.57 (m, 2H) 3.41-3.50 (m, 3H) 3.50-3.61 (m, 1H) 4.18 (t, 2H) 4.84 (br t, 1H) 6.44-6.51 (m, 2H) 6.54 (s, 2H) 7.06-7.14 (m, 2H) 7.32-7.41 (m, 2H) 8.01 (d, 1H) 8.59 (s, 1H)

### Example 39

### 2′-[6-amino-5-(trifluoromethyl)pyridin-3-yl]-N-[(1R)-1-phenylpropyl]-5′,6′-dihydrospiro[pyrrolidine-3,4′-pyrrolo[1,2-b]pyrazole]-1-carboxamide (Mixture of Stereoisomers)

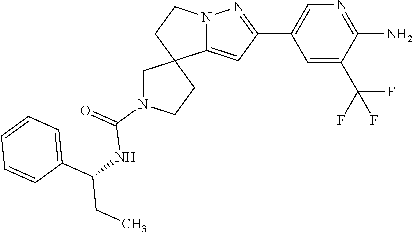 [SMILES: CC[C@@H](NC(=O)N1CCC2(CCn3nc(-c4cnc(N)c(C(F)(F)F)c4)cc32)C1)c1ccccc1]

Example 39 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and (1R)-1-phenylpropan-1-amine (31.0 mg, 229 μmol).

LC-MS (Method 2): Rₜ=1.12 min; MS (ESIpos): m/z=485 [M+H]⁺

¹H NMR (400 MHz, DMSO-d₆) δ ppm 0.80-0.92 (m, 3H) 1.62-1.79 (m, 2H) 2.01-2.12 (m, 2H) 2.52-2.57 (m, 2H) 3.38-3.49 (m, 3H) 3.50-3.65 (m, 1H) 4.18 (t, 2H) 4.54-4.61 (m, 1H) 6.37-6.48 (m, 2H) 6.55 (s, 2H) 7.13-7.23 (m, 1H) 7.24-7.36 (m, 4H) 7.98-8.01 (m, 1H) 8.57 (s, 1H)

### Example 40

### 2′-[6-amino-5-(trifluoromethyl)pyridin-3-yl]-N-{(1S)-1-[3-(trifluoromethyl)phenyl]ethyl}-5′,6′-dihydrospiro[pyrrolidine-3,4′-pyrrolo[1,2-b]pyrazole]-1-carboxamide (Mixture of Stereoisomers)

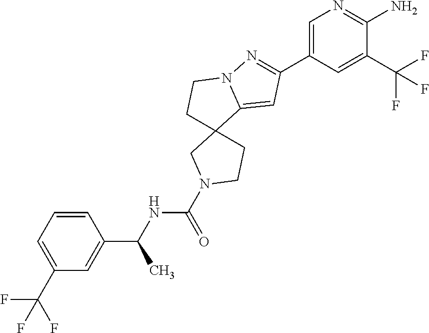 [SMILES: C[C@H](NC(=O)N1CCC2(CCn3nc(-c4cnc(N)c(C(F)(F)F)c4)cc32)C1)c1cccc(C(F)(F)F)c1]

Example 40 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (100 mg, 90% purity, 250 μmol) and (1S)-1-[3-(trifluoromethyl)phenyl]ethan-1-amine (56.8 mg, 300 μmol).

LC-MS (method 1): Rₜ=1.19 min; MS (ESIpos): m/z=539 [M+H]⁺

¹H NMR (400 MHz, DMSO-d₆) δ ppm 1.39 (dd, 3H) 2.08 (br d, 2H) 2.52-2.59 (m, 2H) 3.41-3.51 (m, 3H) 3.51-3.63 (m, 1H) 4.19 (t, 2H) 4.88-4.96 (m, 1H) 6.41-6.46 (m, 1H) 6.55 (s, 2H) 6.63 (dd, 1H) 7.50-7.59 (m, 2H) 7.62-7.73 (m, 2H) 8.00 (s, 1H) 8.55-8.62 (m, 1H)

### Example 41

### 2′-[6-amino-5-(trifluoromethyl)pyridin-3-yl]-N-[(1R)-1-phenylethyl]-5′,6′-dihydrospiro[pyrrolidine-3,4′-pyrrolo[1,2-b]pyrazole]-1-carboxamide (Mixture of Stereoisomers)

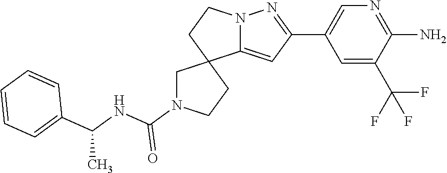 [SMILES: C[C@@H](NC(=O)N1CCC2(CCn3nc(-c4cnc(N)c(C(F)(F)F)c4)cc32)C1)c1ccccc1]

Example 41 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and (1R)-1-phenylethan-1-amine (27.8 mg, 229 μmol).

LC-MS (Method 2): Rt=1.06 min; MS (ESIpos): m/z=471 [M+H]⁺

¹H NMR (400 MHz, DMSO-d₆) δ ppm 1.37 (dd, 3H) 2.02-2.12 (m, 2H) 2.53-2.57 (m, 2H) 3.41-3.51 (m, 3H) 3.52-3.65 (m, 1H) 4.18 (t, 2H) 4.84 (br t, 1H) 6.43-6.50 (m, 2H) 6.55 (s, 2H) 7.14-7.21 (m, 1H) 7.24-7.40 (m, 4H) 8.01 (d, 1H) 8.58 (br s, 1H)

### Example 42

### (rac)-2′-[6-amino-5-(trifluoromethyl) pyridin-3-yl]-N-[2-(pyridin-2-yl)propan-2-yl]-5′,6′-dihydrospiro[pyrrolidine-3,4′-pyrrolo[1,2-b]pyrazole]-1-carboxamide

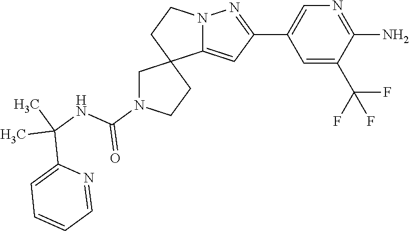 [SMILES: CC(C)(NC(=O)N1CCC2(CCn3nc(-c4cnc(N)c(C(F)(F)F)c4)cc32)C1)c1ccccn1]

Example 42 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and 2-(pyridin-2-yl)propan-2-amine (31.2 mg, 229 μmol).

LC-MS (method 2): Rt=0.72 min; MS (ESIpos): m/z=486 [M+H]⁺

¹H NMR (400 MHz, DMSO-d₆) δ ppm 1.59 (s, 6H) 2.03-2.15 (m, 2H) 2.52-2.59 (m, 2H) 3.42-3.50 (m, 3H) 3.52-3.66 (m, 1H) 4.20 (t, 2H) 6.43 (s, 1H) 6.49 (s, 1H) 6.55 (s, 2H) 7.18 (ddd, 1H) 7.46 (d, 1H) 7.70 (td, 1H) 8.02 (d, 1H) 8.46 (d, 1H) 8.60 (d, 1H)

### Example 43

### 2′-[6-amino-5-(trifluoromethyl)pyridin-3-yl]-N-[(1R)-1-(2-cyanophenyl)ethyl]-5′,6′-dihydrospiro[pyrrolidine-3,4′-pyrrolo[1,2-b]pyrazole]-1-carboxamide (Mixture of Stereoisomers)

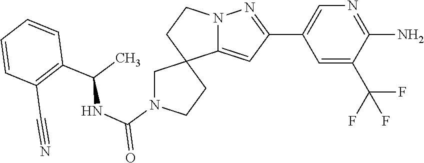 [SMILES: C[C@@H](NC(=O)N1CCC2(CCn3nc(-c4cnc(N)c(C(F)(F)F)c4)cc32)C1)c1ccccc1C#N]

Example 43 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and 2-[(1R)-1-aminoethyl]benzonitrile (33.5 mg, 229 μmol).

LC-MS (Method 2): Rt=1.01 min; MS (ESIpos): m/z=496 [M+H]⁺

¹H NMR (400 MHz, DMSO-d₆) δ ppm 1.32-1.44 (m, 3H) 2.01-2.14 (m, 2H) 3.39-3.51 (m, 3H) 3.52-3.65 (m, 1H) 4.19 (t, 2H) 5.02-5.10 (m, 1H) 6.42-6.49 (m, 1H) 6.56 (br s, 2H) 6.76 (dd, 1H) 7.34-7.43 (m, 1H) 7.59-7.77 (m, 3H) 8.01 (t, 1H) 8.58 (s, 1H)

### Example 44

### 2′-[6-amino-5-(trifluoromethyl)pyridin-3-yl]-N-[1-(1,3,5-trimethyl-1H-pyrazol-4-yl)ethyl]-5′,6′-dihydrospiro[pyrrolidine-3,4′-pyrrolo[1,2-b]pyrazole]-1-carboxamide (Diastereomeric Mixture)

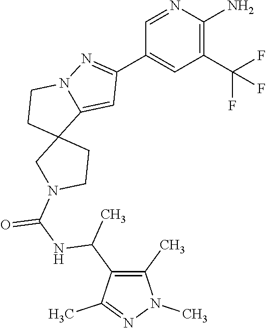 [SMILES: Cc1nn(C)c(C)c1C(C)NC(=O)N1CCC2(CCn3nc(-c4cnc(N)c(C(F)(F)F)c4)cc32)C1]

Example 44 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and (rac)-1-(1,3,5-trimethyl-1H-pyrazol-4-yl)ethan-1-amine (47.9 mg, 313 μmol).

LC-MS (method 1): Rₜ=0.97 min; MS (ESIpos): m/z=503 [M+H]⁺

¹H-NMR (400 MHz, DMSO-d6): δ [ppm]=1.33 and 1.34 (2d, 3H), 1.99-2.08 (m, 2H), 2.11 and 2.13 (2s, 3H), 2.18 and 2.19 (2s, 3H), 3.35-3.59 (m, 7H), 4.17 (t, 2H), 4.70 (quin, 1H), 6.08 and 6.08 (2d, 1H), 6.44 and 6.44 (2s, 1H), 6.54 (s, 2H), 8.00 (s, 1H), 8.57 and 8.58 (2d, 1H).

### Example 45

### 2′-[6-amino-5-(trifluoromethyl)pyridin-3-yl]-N-[(1R)-2,3-dihydro-1H-inden-1-yl]-5′,6′-dihydrospiro[pyrrolidine-3,4′-pyrrolo[1,2-b]pyrazole]-1-carboxamide (Mixture of Stereoisomers)

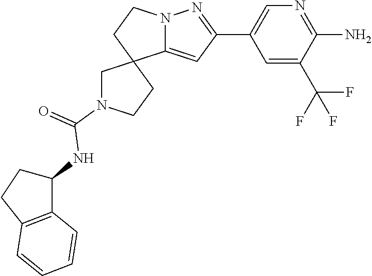 [SMILES: Nc1ncc(-c2cc3n(n2)CCC32CCN(C(=O)N[C@@H]3CCc4ccccc43)C2)cc1C(F)(F)F]

Example 45 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and (1R)-2,3-dihydro-1H-inden-1-amine (30.5 mg, 229 μmol).

LC-MS (method 2): Rₜ=1.10 min; MS (ESIpos): m/z=484 [M+H]⁺

¹H NMR (400 MHz, DMSO-d₆) δ ppm 1.79-1.91 (m, 1H) 1.98-2.13 (m, 2H) 2.33-2.45 (m, 1H) 2.52-2.58 (m, 2H) 2.74-2.82 (m, 1H) 3.42-3.53 (m, 3H) 3.53-3.65 (m, 1H) 4.19 (t, 2H) 5.22 (q, 1H) 6.45-6.58 (m, 4H) 7.12-7.27 (m, 4H) 7.95 (s, 1H) 8.00-8.04 (m, 1H) 8.60 (s, 1H)

### Example 46

### 2′-[6-amino-5-(trifluoromethyl)pyridin-3-yl]-N-[(1R)-1-(2-cyanopyridin-4-yl)ethyl]-5′,6′-dihydrospiro[pyrrolidine-3,4′-pyrrolo[1,2-b]pyrazole]-1-carboxamide (Mixture of Stereoisomers)

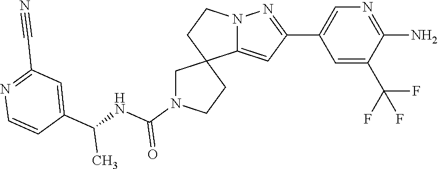 [SMILES: C[C@@H](NC(=O)N1CCC2(CCn3nc(-c4cnc(N)c(C(F)(F)F)c4)cc32)C1)c1ccnc(C#N)c1]

Example 46 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and 4-[(1R)-1-aminoethyl]pyridine-2-carbonitrile (33.7 mg, 229 μmol).

LC-MS (Method 2): Rt=0.94 min; MS (ESIpos): m/z=497 [M+H]⁺

¹H NMR (400 MHz, DMSO-d₆) δ ppm 1.02-1.08 (m, 1H) 1.31-1.44 (m, 3H) 2.02-2.14 (m, 2H) 2.52-2.58 (m, 2H) 3.36-3.52 (m, 3H) 3.53 (br d, 1H) 4.17-4.22 (m, 1H) 4.82-4.92 (m, 1H) 6.49 (d, 1H) 6.54 (s, 2H) 6.68 (dd, 1H) 7.62-7.72 (m, 1H) 7.99-8.03 (m, 2H) 8.59 (d, 1H) 8.64-8.70 (m, 1H)

### Example 47

### 2′-[6-amino-5-(trifluoromethyl)pyridin-3-yl]-N-[1-(1-ethyl-3,5-dimethyl-1H-pyrazol-4-yl)ethyl]-5′,6′-dihydrospiro[pyrrolidine-3,4′-pyrrolo[1,2-b]pyrazole]-1-carboxamide (Diastereometric Mixture)

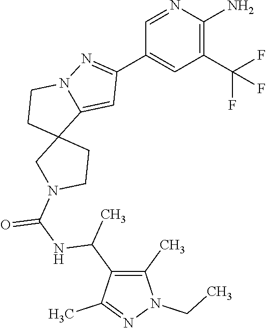 [SMILES: CCn1nc(C)c(C(C)NC(=O)N2CCC3(CCn4nc(-c5cnc(N)c(C(F)(F)F)c5)cc43)C2)c1C]

Example 47 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and (rac)-1-(1-ethyl-3,5-dimethyl-1H-pyrazol-4-yl)ethan-1-amine (52.3 mg, 313 μmol).

LC-MS (method 1): Rₜ=0.99 min; MS (ESIpos): m/z=517 [M+H]⁺

¹H-NMR (400 MHz, DMSO-d6) δ[ppm]: 1.194 (3.80), 1.204 (4.08), 1.212 (9.50), 1.223 (9.14), 1.230 (4.12), 1.240 (3.95), 1.324 (5.19), 1.334 (5.68), 1.342 (5.58), 1.352 (5.45), 2.017 (1.28), 2.031 (1.98), 2.042 (2.29), 2.059 (1.01), 2.124 (15.13), 2.142 (15.21), 2.190 (16.00), 2.206 (15.73), 2.322 (0.44), 2.327 (0.61), 2.332 (0.45), 2.523 (2.49), 2.532 (2.39), 2.664 (0.42), 2.669 (0.62), 2.673 (0.45), 3.354 (0.64), 3.366 (1.46), 3.372 (0.96), 3.378 (1.38), 3.391 (2.62), 3.403 (2.64), 3.423 (2.62), 3.431 (2.30), 3.447 (1.04), 3.457 (1.11), 3.472 (0.42), 3.490 (0.71), 3.504 (0.81), 3.521 (0.81), 3.529 (0.62), 3.546 (0.50), 3.855 (0.94), 3.872 (3.28), 3.886 (3.50), 3.890 (3.46), 3.903 (3.21), 3.922 (0.91), 4.155 (2.59), 4.173 (4.99), 4.190 (2.52), 4.698 (1.56), 4.715 (2.34), 4.732 (1.53), 6.063 (1.80), 6.076 (1.70), 6.080 (1.68), 6.440 (4.08), 6.447 (7.13), 6.540 (6.24), 8.003 (3.43), 8.576 (3.51).

### Example 48

### (rac)-2′-[6-amino-5-(trifluoromethyl)pyridin-3-yl]-N-[1-(pyridin-2-yl)cyclobutyl]-5′,6′-dihydrospiro[pyrrolidine-3,4′-pyrrolo[1,2-b]pyrazole]-1-carboxamide

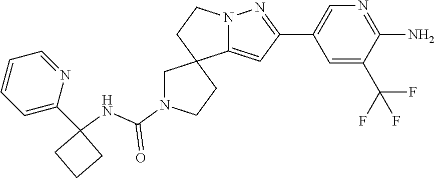 [SMILES: Nc1ncc(-c2cc3n(n2)CCC32CCN(C(=O)NC3(c4ccccn4)CCC3)C2)cc1C(F)(F)F]

Example 48 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and 1-(pyridin-2-yl)cyclobutan-1-amine (34.0 mg, 229 μmol).

LC-MS (Method 2): Rt=0.77 min; MS (ESIpos): m/z=498 [M+H]⁺

¹H NMR (400 MHz, DMSO-d₆) δ ppm 1.23 (s, 1H) 1.85-2.15 (m, 4H) 2.52-2.61 (m, 5H) 3.43-3.52 (m, 3H) 3.53-3.67 (m, 1H) 4.20 (t, 2H) 6.48 (s, 1H) 6.56 (s, 2H) 6.79 (s, 1H) 7.17 (ddd, 1H) 7.37 (d, 1H) 7.67 (td, 1H) 8.01 (d, 1H) 8.53 (d, 1H) 8.59 (d, 1H)

### Example 49

### 2′-[6-amino-5-(trifluoromethyl)pyridin-3-yl]-N-[1-(1,3,4-trimethyl-1H-pyrazol-5-yl)ethyl]-5′,6′-dihydrospiro[pyrrolidine-3,4′-pyrrolo[1,2-b]pyrazole]-1-carboxamide (Diastereometric Mixture)

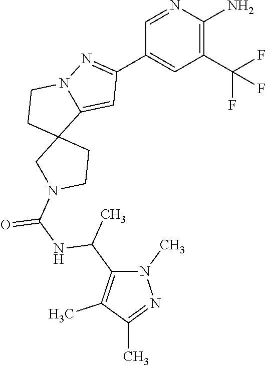 [SMILES: Cc1nn(C)c(C(C)NC(=O)N2CCC3(CCn4nc(-c5cnc(N)c(C(F)(F)F)c5)cc43)C2)c1C]

Example 49 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (33.0 mg, 91.8 μmol) and (rac)-1-(1,3,4-trimethyl-1H-pyrazol-5-yl)ethan-1-amine (21.1 mg, 138 μmol).

LC-MS (method 1): Rₜ=0.98 min; MS (ESIpos): m/z=503 [M+H]⁺

¹H-NMR (400 MHz, DMSO-d6) δ[ppm]: 1.383 (4.24), 1.389 (3.94), 1.401 (4.53), 1.408 (3.75), 1.919 (6.99), 1.956 (16.00), 1.972 (9.50), 2.049 (2.01), 2.327 (0.85), 2.539 (2.84), 2.669 (0.91), 3.372 (2.06), 3.398 (2.16), 3.404 (2.49), 3.424 (0.80), 3.452 (2.99), 3.477 (1.62), 3.538 (0.58), 3.564 (0.43), 3.695 (7.90), 3.708 (9.89), 4.160 (1.82), 4.177 (3.49), 4.194 (1.82), 4.892 (0.78), 4.909 (1.11), 4.926 (0.78), 6.434 (3.72), 6.440 (4.68), 6.452 (1.75), 6.545 (4.85), 8.003 (2.94), 8.575 (2.56).

### Example 50

### 2′-[6-amino-5-(trifluoromethyl)pyridin-3-yl]-N-[(1R)-1-(4-cyanopyridin-2-yl)ethyl]-5′,6′-dihydrospiro[pyrrolidine-3,4′-pyrrolo[1,2-b]pyrazole]-1-carboxamide (Mixture of Stereoisomers)

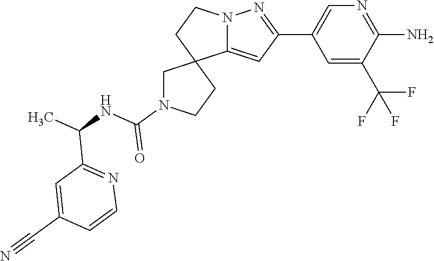 [SMILES: C[C@@H](NC(=O)N1CCC2(CCn3nc(-c4cnc(N)c(C(F)(F)F)c4)cc32)C1)c1cc(C#N)ccn1]

Example 50 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and 2-[(1R)-1-aminoethyl]pyridine-4-carbonitrile hydrochloride (42.1 mg, 229 μmol).

LC-MS (Method 2): Rt=0.92 min; MS (ESIpos): m/z=497 [M+H]⁺

¹H NMR (400 MHz, DMSO-d₆) δ ppm 1.41 (dd, 3H) 2.02-2.13 (m, 2H) 2.52-2.57 (m, 2H) 3.37-3.51 (m, 3H) 3.52-3.65 (m, 1H) 4.19 (t, 2H) 4.88-4.97 (m, 1H) 6.47 (s, 1H) 6.54 (s, 2H) 6.63 (dd, 1H) 8.01 (t, 1H) 8.21-8.29 (m, 1H) 8.58 (s, 1H) 8.79-8.88 (m, 2H)

### Example 51

### 2′-[6-amino-5-(trifluoromethyl)pyridin-3-yl]-N-[(1R)-1-(pyridin-3-yl)ethyl]-5′,6′-dihydrospiro[pyrrolidine-3,4′-pyrrolo[1,2-b]pyrazole]-1-carboxamide (Mixture of Stereoisomers)

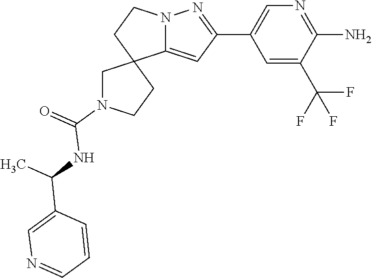 [SMILES: C[C@@H](NC(=O)N1CCC2(CCn3nc(-c4cnc(N)c(C(F)(F)F)c4)cc32)C1)c1cccnc1]

Example 51 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and (1R)-1-(pyridin-3-yl)ethan-1-amine hydrochloride (61.0 mg, 313 μmol).

LC-MS (method 2): Rₜ=0.71 min; MS (ESIpos): m/z=472 [M+H]⁺

¹H NMR (400 MHz, DMSO-d₆) δ ppm 1.40 (dd, 3H) 2.01-2.12 (m, 2H) 2.53-2.57 (m, 2H) 3.41-3.49 (m, 3H) 3.50-3.62 (m, 1H) 4.16-4.22 (m, 2H) 4.83-4.91 (m, 1H) 6.43-6.48 (m, 1H) 6.51-6.60 (m, 3H) 7.32 (td, 1H) 7.71-7.77 (m, 1H) 8.01 (s, 1H) 8.38-8.43 (m, 1H) 8.53-8.60 (m, 2H)

### Example 52

### (rac)-2′-[6-amino-5-(trifluoromethyl)pyridin-3-yl]-N-[2-(1-methyl-1H-1,2,3-triazol-4-yl)propan-2-yl]-5′,6′-dihydrospiro[pyrrolidine-3,4′-pyrrolo[1,2-b]pyrazole]-1-carboxamide

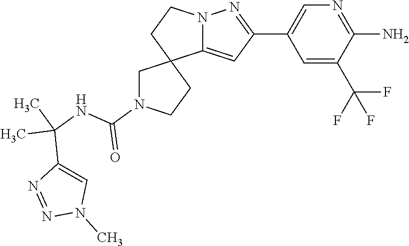 [SMILES: Cn1cc(C(C)(C)NC(=O)N2CCC3(CCn4nc(-c5cnc(N)c(C(F)(F)F)c5)cc43)C2)nn1]

Example 52 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and 2-(1-methyl-1H-1,2,3-triazol-4-yl)propan-2-amine hydrochloride (48.9 mg, 229 μmol).

LC-MS (Method 2): Rt=0.85 min; MS (ESIpos): m/z=490 [M+H]⁺

¹H NMR (400 MHz, DMSO-d₆) δ ppm 1.60 (d, 6H) 2.00-2.11 (m, 2H) 2.53-2.56 (m, 2H) 3.36-3.46 (m, 3H) 3.48-3.58 (m, 1H) 3.97 (s, 3H) 4.18 (t, 2H) 5.92 (s, 1H) 6.48 (s, 1H) 6.54 (s, 2H) 7.80 (s, 1H) 8.02 (d, 1H) 8.60 (d, 1H)

### Example 53

### (rac)-2′-[6-amino-5-(trifluoromethyl)pyridin-3-yl]-N-{[3-(trifluoromethyl)phenyl]methyl}-5′,6′-dihydrospiro[pyrrolidine-3,4′-pyrrolo[1,2-b]pyrazole]-1-carboxamide

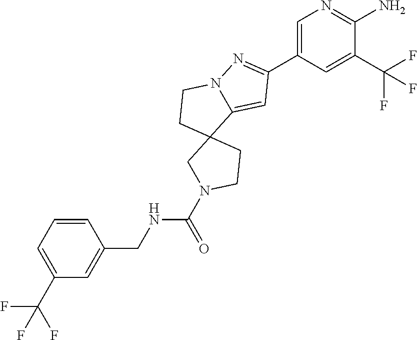 [SMILES: Nc1ncc(-c2cc3n(n2)CCC32CCN(C(=O)NCc3cccc(C(F)(F)F)c3)C2)cc1C(F)(F)F]

Example 53 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (100 mg, 90% purity, 250 μmol) and 1-[3-(trifluoromethyl)phenyl]methanamine (52.6 mg, 300 μmol).

LC-MS (method 1): Rₜ=1.15 min; MS (ESIpos): m/z=525 [M+H]⁺

¹H NMR (400 MHz, DMSO-d₆) δ ppm 2.05-2.14 (m, 2H) 2.53-2.59 (m, 2H) 3.42-3.51 (m, 3H) 3.52-3.62 (m, 1H) 4.19 (t, 2H) 4.33 (d, 2H) 6.45 (s, 1H) 6.55 (s, 2H) 6.93 (t, 1H) 7.52-7.63 (m, 4H) 8.01 (d, 1H) 8.59 (d, 1H)

### Example 54

### 2′-[6-amino-5-(trifluoromethyl)pyridin-3-yl]-N-[1-(1-phenyl-1H-pyrazol-4-yl)ethyl]-5′,6′-dihydrospiro[pyrrolidine-3,4′-pyrrolo[1,2-b]pyrazole]-1-carboxamide (Diastereometric Mixture)

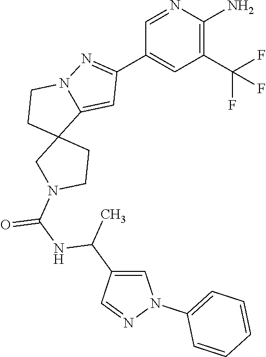 [SMILES: CC(NC(=O)N1CCC2(CCn3nc(-c4cnc(N)c(C(F)(F)F)c4)cc32)C1)c1cnn(-c2ccccc2)c1]

Example 54 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and (rac)-1-(1-phenyl-1H-pyrazol-4-yl)ethan-1-amine (58.5 mg, 313 μmol).

LC-MS (method 1): Rₜ=1.10 min; MS (ESIpos): m/z=537 [M+H]⁺

¹H-NMR (400 MHz, DMSO-d6): δ [ppm]=1.43 and 1.44 (2d, 3H), 2.01-2.13 (m, 2H), 2.52-2.57 (m, 2H), 3.40-3.51 (m, 3H), 3.53-3.61 (m, 1H), 4.19 (t, 2H), 4.91 (quin, 1H), 6.34 and 6.36 (2s, 1H), 6.48 and 6.49 (2s, 1H), 6.54 (s, 2H), 7.24-7.30 (m, 1H), 7.43-7.51 (m, 2H), 7.66 and 7.69 (2s, 1H), 7.76-7.82 (m, 2H), 8.02 (s, 1H), 8.32 and 8.34 (2s, 1H), 8.60 (d, 1H).

### Example 55

### 2′-[6-amino-5-(trifluoromethyl)pyridin-3-yl]-N-[(3S)-2,3-dihydro-1-benzofuran-3-yl]-5′,6′-dihydrospiro[pyrrolidine-3,4′-pyrrolo[1,2-b]pyrazole]-1-carboxamide (Mixture of Stereoisomers)

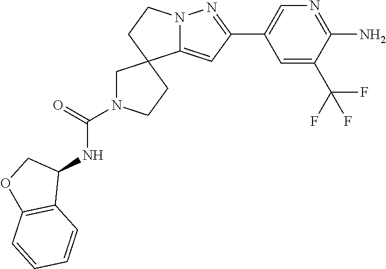 [SMILES: Nc1ncc(-c2cc3n(n2)CCC32CCN(C(=O)N[C@@H]3COc4ccccc43)C2)cc1C(F)(F)F]

Example 55 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and (3S)-2,3-dihydro-1-benzofuran-3-amine (31.0 mg, 229 μmol).

LC-MS (Method 2): Rt=1.02 min; MS (ESIpos): m/z=485 [M+H]⁺

¹H NMR (400 MHz, DMSO-d₆) δ ppm 2.01-2.10 (m, 2H) 2.52-2.56 (m, 2H) 3.40-3.50 (m, 3H) 3.50-3.62 (m, 1H) 4.17 (t, 2H) 4.24 (dt, 1H) 4.66 (td, 1H) 5.43-5.50 (m, 1H) 6.47 (d, 1H) 6.54 (s, 2H) 6.73-6.78 (m, 1H) 6.80 (d, 1H) 6.84-6.91 (m, 1H) 7.18 (tdd, 1H) 7.33 (t, 1H) 8.01 (s, 1H) 8.59 (d, 1H)

### Example 56

### 2′-[6-amino-5-(trifluoromethyl)pyridin-3-yl]-N-[(1R)-1-(6-cyanopyridin-3-yl)ethyl]-5′,6′-dihydrospiro[pyrrolidine-3,4′-pyrrolo[1,2-b]pyrazole]-1-carboxamide (Mixture of Stereoisomers)

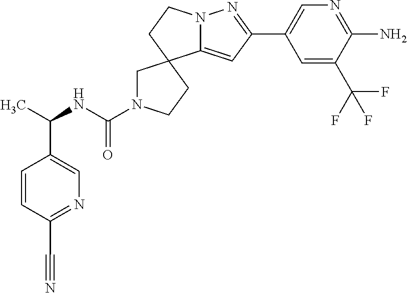 [SMILES: C[C@@H](NC(=O)N1CCC2(CCn3nc(-c4cnc(N)c(C(F)(F)F)c4)cc32)C1)c1ccc(C#N)nc1]

Example 56 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and 5-[(1R)-1-aminoethyl]pyridine-2-carbonitrile (33.7 mg, 229 μmol).

LC-MS (Method 2): Rt=0.94 min; MS (ESIpos): m/z=497 [M+H]⁺

¹H NMR (400 MHz, DMSO-d₆) δ ppm 1.41 (dd, 3H) 1.99-2.15 (m, 2H) 2.52-2.57 (m, 2H) 3.47-3.54 (m, 3H) 3.55-3.63 (m, 1H) 4.18 (br t, 2H) 4.92 (quin, 1H) 6.47 (s, 1H) 6.53 (s, 2H) 6.72 (dd, 1H) 7.95-7.99 (m, 2H) 8.01 (s, 1H) 8.58 (d, 1H) 8.73 (d, 1H)

### Example 57

### 2′-[6-amino-5-(trifluoromethyl)pyridin-3-yl]-N-{(1S)-1-[1-methyl-3-(trifluoromethyl)-1H-pyrazol-4-yl]ethyl}-5′,6′-dihydrospiro[pyrrolidine-3,4′-pyrrolo[1,2-b]pyrazole]-1-carboxamide (Mixture of Stereoisomers)

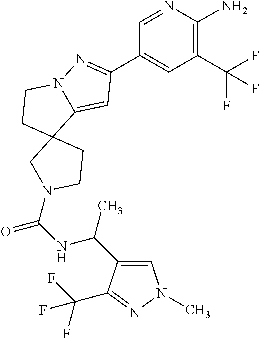 [SMILES: CC(NC(=O)N1CCC2(CCn3nc(-c4cnc(N)c(C(F)(F)F)c4)cc32)C1)c1cn(C)nc1C(F)(F)F]

Example 57 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (31.3 mg, 87.1 μmol) and 1): 1-[1-methyl-3-(trifluoromethyl)-1H-pyrazol-4-yl]ethan-1-amine hydrochloride (30.0 mg, 131 μmol).

¹H-NMR (400 MHz, DMSO-d6): δ [ppm]=1.34 and 1.34 (2d, 3H), 2.01-2.11 (m, 2H), 3.36-3.58 (m, 5H), 3.85-3.88 (m, 4H), 4.18 (t, 2H), 4.89-5.01 (m, 1H), 6.36 and 6.38 (2s, 1H), 6.42-6.47 (m, 1H), 6.55 (br d, 2H), 7.80 and 7.85 (2s, 1H), 7.99-8.02 (m, 1H), 8.57-8.61 (m, 1H).

LC-MS (method 1): Rₜ=1.04 min; MS (ESIneg): m/z=541 [M−H]⁻

### Example 58

### 2′-[6-amino-5-(trifluoromethyl)pyridin-3-yl]-N-[(1R)-1-(pyridin-2-yl)ethyl]-5′,6′-dihydrospiro[pyrrolidine-3,4′-pyrrolo[1,2-b]pyrazole]-1-carboxamide (Mixture of Stereoisomers)

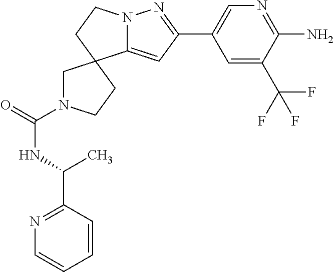 [SMILES: C[C@@H](NC(=O)N1CCC2(CCn3nc(-c4cnc(N)c(C(F)(F)F)c4)cc32)C1)c1ccccn1]

Example 58 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and (1R)-1-(pyridin-2-yl)ethan-1-amine (28.0 mg, 229 μmol).

LC-MS (method 2): Rt=0.75 min; MS (ESIpos): m/z=472 [M+H]⁺

¹H NMR (400 MHz, DMSO-d₆) δ ppm 1.39 (dd, 3H) 2.00-2.14 (m, 2H) 2.54-2.59 (m, 2H) 3.44-3.51 (m, 3H) 3.51-3.64 (m, 1H) 4.15-4.23 (m, 2H) 4.84-4.92 (m, 1H) 6.47-6.49 (m, 1H) 6.55 (s, 2H) 7.19-7.25 (m, 1H) 7.39 (dd, 1H) 7.73 (dt, 1H) 7.73 (dt, 1H) 8.01 (d, 1H) 8.48 (ddt, 1H) 8.59 (s, 1H)

### Example 59

### (rac)-2′-[6-amino-5-(trifluoromethyl)pyridin-3-yl]-N-benzyl-5′,6′-dihydrospiro[pyrrolidine-3,4′-pyrrolo[1,2-b]pyrazole]-1-carboxamide

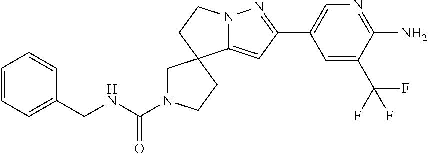 [SMILES: Nc1ncc(-c2cc3n(n2)CCC32CCN(C(=O)NCc3ccccc3)C2)cc1C(F)(F)F]

Example 59 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and 1-phenylmethanamine (27 μL, 250 μmol).

LC-MS (Method 2): Rt=1.01 min; MS (ESIpos): m/z=457 [M+H]⁺

¹H NMR (400 MHz, DMSO-d₆) δ ppm 2.03-2.14 (m, 2H) 3.46 (s, 3H) 3.52-3.60 (m, 1H) 4.19 (t, 2H) 4.25 (d, 2H) 6.47 (s, 1H) 6.55 (s, 2H) 6.80 (t, 1H) 7.20 (s, 1H) 7.24-7.32 (m, 4H) 8.02 (d, 1H) 8.59 (d, 1H)

### Example 60

### 2′-[6-amino-5-(trifluoromethyl)pyridin-3-yl]-N-[(1S)-1-phenylethyl]-5′,6′-dihydrospiro[pyrrolidine-3,4′-pyrrolo[1,2-b]pyrazole]-1-carboxamide (Mixture of Stereoisomers)

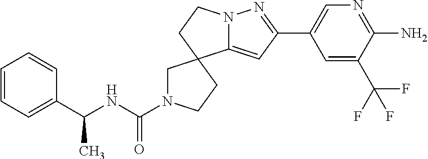 [SMILES: C[C@H](NC(=O)N1CCC2(CCn3nc(-c4cnc(N)c(C(F)(F)F)c4)cc32)C1)c1ccccc1]

Example 60 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and (1S)-1-phenylethan-1-amine (27.8 mg, 229 μmol).

LC-MS (Method 1): Rt=1.08 min; MS (ESIpos): m/z=471 [M+H]⁺

¹H NMR (400 MHz, DMSO-d₆) δ ppm 1.37 (dd, 3H) 1.77 (s, 1H) 2.00-2.13 (m, 2H) 2.52-2.56 (m, 2H) 3.56 (br s, 3H) 4.18 (t, 2H) 4.84 (quin, 1H) 6.43-6.49 (m, 2H) 6.55 (s, 2H) 7.14-7.23 (m, 1H) 7.24-7.38 (m, 4H) 8.01 (d, 1H) 8.58 (t, 1H)

### Example 61

### 2′-[6-amino-5-(trifluoromethyl)pyridin-3-yl]-N-[(1S)-1-(1,2-oxazol-3-yl)ethyl]-5′,6′-dihydrospiro[pyrrolidine-3,4′-pyrrolo[1,2-b]pyrazole]-1-carboxamide (Mixture of Stereoisomers)

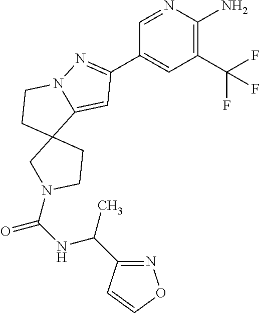 [SMILES: CC(NC(=O)N1CCC2(CCn3nc(-c4cnc(N)c(C(F)(F)F)c4)cc32)C1)c1ccon1]

Example 61 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and 1): 1-(1,2-oxazol-3-yl)ethan-1-amine hydrochloride (46.5 mg, 313 μmol).

¹H-NMR (400 MHz, DMSO-d6): δ [ppm]=1.42 and 1.43 (2d, 3H), 2.01-2.13 (m, 2H), 2.52-2.57 (m, 2H), 3.40-3.50 (m, 3H), 3.52-3.60 (m, 1H), 4.19 (t, 2H), 5.01 (quin, 1H), 6.48 (d, 1H), 6.50-6.56 (m, 3H), 6.58 and 6.60 (2d, 1H), 8.02 (d, 1H), 8.59 (d, 1H), 8.76 and 8.77 (2d, 1H).

LC-MS (method 1): Rₜ=0.93 min; MS (ESIpos): m/z=462 [M+H]⁺

### Example 62

### 2′-[6-amino-5-(trifluoromethyl)pyridin-3-yl]-N-[(1S)-1-(1,5-dimethyl-1H-pyrazol-4-yl)ethyl]-5′,6′-dihydrospiro[pyrrolidine-3,4′-pyrrolo[1,2-b]pyrazole]-1-carboxamide (Diastereomeric Mixture)

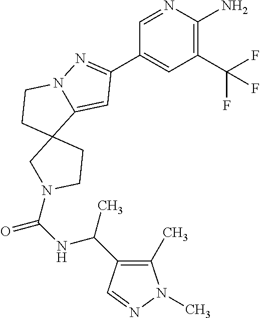 [SMILES: Cc1c(C(C)NC(=O)N2CCC3(CCn4nc(-c5cnc(N)c(C(F)(F)F)c5)cc43)C2)cnn1C]

Example 62 was prepared in analogy to Example 31 using (rac)-5-[−5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and 1-(1,5-dimethyl-1H-pyrazol-4-yl)ethan-1-amine (43.5 mg, 313 μmol).

¹H-NMR (400 MHz, DMSO-d6): δ [ppm]=1.32 and 1.34 (2d, 3H), 1.98-2.10 (m, 2H), 2.18 and 2.19 (2s, 3H), 3.35-3.45 (m, 3H), 3.46-3.56 (m, 1H), 3.65 and 3.66 (2s, 3H), 4.17 (t, 2H), 4.71-4.80 (m, 1H), 6.14 and 6.16 (2d, 1H), 6.44 and 6.45 (2s, 1H), 6.54 (s, 2H), 7.28 and 7.30 (2s, 1H), 8.01 (d, 1H), 8.58 (d, 1H).

LC-MS (method 1): Rₜ=0.92 min; MS (ESIpos): m/z=489 [M+H]⁺

### Example 63

### (rac)-2′-[6-amino-5-(trifluoromethyl)pyridin-3-yl]-N-[2-(oxan-4-yl)propan-2-yl]-5′,6′-dihydrospiro[pyrrolidine-3,4′-pyrrolo[1,2-b]pyrazole]-1-carboxamide

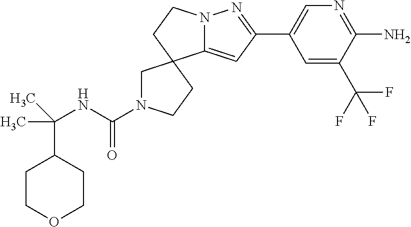 [SMILES: CC(C)(NC(=O)N1CCC2(CCn3nc(-c4cnc(N)c(C(F)(F)F)c4)cc32)C1)C1CCOCC1]

Example 63 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and 2-(oxan-4-yl)propan-2-amine hydrochloride (41.2 mg, 229 μmol).

LC-MS (Method 2): Rₜ=1.00 min; MS (ESIpos): m/z=494 [M+H]⁺

¹H NMR (400 MHz, DMSO-d₆) δ ppm 1.18 (d, 6H) 1.20-1.32 (m, 2H) 1.46 (br s, 2H) 1.98-2.10 (m, 2H) 2.22-2.32 (m, 1H) 2.53-2.58 (m, 1H) 3.20 (br t, 2H) 3.35-3.43 (m, 3H) 3.46-3.56 (m, 1H) 3.83-3.90 (m, 2H) 4.18 (t, 2H) 5.14 (s, 1H) 6.43 (s, 1H) 6.55 (s, 2H) 8.00 (d, 1H) 8.57 (d, 1H)

### Example 64

### 2′-[6-amino-5-(trifluoromethyl)pyridin-3-yl]-N-[(1R)-1-(pyridin-4-yl)ethyl]-5′,6′-dihydrospiro[pyrrolidine-3,4′-pyrrolo[1,2-b]pyrazole]-1-carboxamide (Mixture of Stereoisomers)

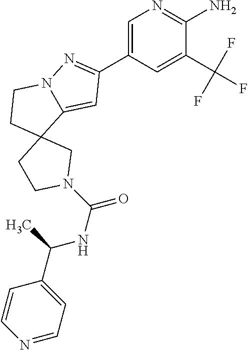 [SMILES: C[C@@H](NC(=O)N1CCC2(CCn3nc(-c4cnc(N)c(C(F)(F)F)c4)cc32)C1)c1ccncc1]

Example 64 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (50.0 mg, 139 μmol) and (1R)-1-(pyridin-4-yl)ethan-1-amine hydrochloride (32.5 mg, 167 μmol).

¹H NMR (400 MHz, DMSO-d₆) δ ppm 1.37 (dd, 3H) 2.02-2.14 (m, 2H) 2.52-2.58 (m, 2H) 3.44-3.52 (m, 3H) 3.54-3.65 (m, 1H) 4.19 (t, 2H) 4.77-4.86 (m, 1H) 6.45-6.51 (m, 1H) 6.51-6.62 (m, 3H) 7.26-7.36 (m, 2H) 8.02 (d, 1H) 8.44-8.49 (m, 2H) 8.59 (s, 1H)

LC-MS (method 1): Rₜ=0.89 min; MS (ESIneg): m/z=470 [M−H]⁻

### Example 65

### 2′-[6-amino-5-(trifluoromethyl)pyridin-3-yl]-N-[(1S)-1-(1-ethyl-5-methyl-1H-pyrazol-4-yl)ethyl]-5′,6′-dihydrospiro[pyrrolidine-3,4′-pyrrolo[1,2-b]pyrazole]-1-carboxamide (Diastereomeric Mixture)

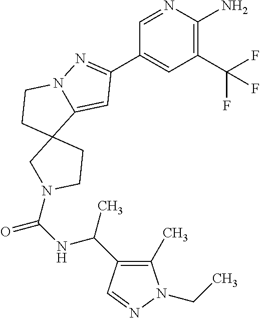 [SMILES: CCn1ncc(C(C)NC(=O)N2CCC3(CCn4nc(-c5cnc(N)c(C(F)(F)F)c5)cc43)C2)c1C]

Example 65 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and 1-(1-ethyl-5-methyl-1H-pyrazol-4-yl)ethan-1-amine (47.9 mg, 313 μmol).

¹H-NMR (400 MHz, DMSO-d6): δ [ppm]=1.22-1.28 (m, 3H), 1.33 and 1.34 (2d, 3H), 1.98-2.10 (m, 2H), 2.19 and 2.20 (2s, 3H), 3.36-3.45 (m, 3H), 3.46-3.56 (m, 1H), 3.93-4.03 (m, 2H), 4.17 (t, 2H), 4.77 (quin, 1H), 6.15 and 6.17 (2d, 1H), 6.45 (d, 1H), 6.54 (s, 2H), 7.32 (d, 1H), 8.01 (d, 1H), 8.58 (d, 1H).

LC-MS (method 1): Rₜ=0.97 min; MS (ESIpos): m/z=503 [M+H]⁺

### Example 66

### (rac)-2′-[6-amino-5-(trifluoromethyl)pyridin-3-yl]-N-[(pyridin-3-yl)methyl]-5′,6′-dihydrospiro[pyrrolidine-3,4′-pyrrolo[1,2-b]pyrazole]-1-carboxamide

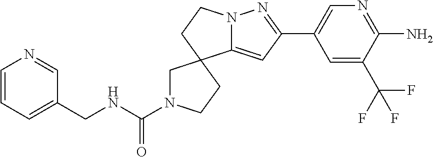 [SMILES: Nc1ncc(-c2cc3n(n2)CCC32CCN(C(=O)NCc3cccnc3)C2)cc1C(F)(F)F]

Example 66 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and 1-(pyridin-3-yl)methanamine (24.8 mg, 229 μmol).

LC-MS (Method 2): Rt=0.86 min; MS (ESIpos): m/z=458 [M+H]⁺

¹H NMR (400 MHz, DMSO-d₆) δ ppm 2.02-2.14 (m, 2H) 2.53-2.59 (m, 2H) 3.36-3.58 (m, 4H) 4.19 (t, 2H) 4.26 (d, 2H) 6.47 (s, 1H) 6.54 (s, 2H) 6.87 (t, 1H) 7.32 (ddd, 1H) 7.68 (dt, 1H) 8.02 (d, 1H) 8.42 (dd, 1H) 8.50 (d, 1H) 8.59 (d, 1H)

### Example 67

### 2′-[6-amino-5-(trifluoromethyl)pyridin-3-yl]-N-[(1S)-2-methoxy-1-phenylethyl]-5′,6′-dihydrospiro[pyrrolidine-3,4′-pyrrolo[1,2-b]pyrazole]-1-carboxamide (Mixture of Stereoisomers)

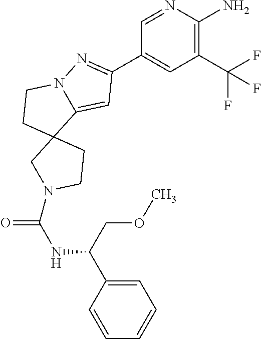 [SMILES: COC[C@@H](NC(=O)N1CCC2(CCn3nc(-c4cnc(N)c(C(F)(F)F)c4)cc32)C1)c1ccccc1]

Example 67 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and (1S)-2-methoxy-1-phenylethan-1-amine (47.3 mg, 313 μmol).

LC-MS (Method 2): Rt=1.03 min; MS (ESIpos): m/z=501 [M+H]⁺ 1H-NMR (400 MHz, DMSO-d6): δ [ppm]=2.03-2.11 (m, 2H), 2.54-2.58 (m, 2H), 3.24 and 3.26 (2d, 3H), 3.41-3.63 (m, 6H), 4.19 (t, 2H), 4.91-4.99 (m, 1H), 6.42-6.50 (m, 2H), 6.55 (s, 2H), 7.17-7.24 (m, 1H), 7.25-7.39 (m, 4H), 8.00 (s, 1H), 8.58 (s, 1H).

### Example 68

### (rac)-2′-[6-amino-5-(trifluoromethyl)pyridin-3-yl]-N-{[3-(trifluoromethyl)bicyclo[1.1.1]pentan-1-yl]methyl}-5′,6′-dihydrospiro[pyrrolidine-3,4′-pyrrolo[1,2-b]pyrazole]-1-carboxamide

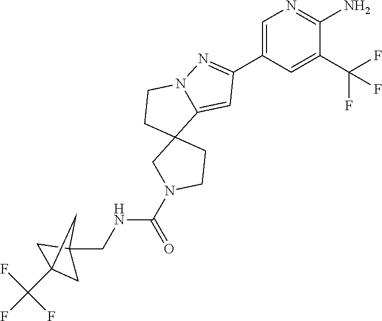 [SMILES: Nc1ncc(-c2cc3n(n2)CCC32CCN(C(=O)NCC34CC(C(F)(F)F)(C3)C4)C2)cc1C(F)(F)F]

Example 68 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and 1-[3-(trifluoromethyl)bicyclo[1.1.1]pentan-1-yl]methanamine hydrochloride (50.4 mg, 250 μmol).

LC-MS (Method 1): Rt=1.14 min; MS (ESIpos): m/z=515 [M+H]⁺

¹H NMR (400 MHz, DMSO-d₆) δ ppm 1.82 (s, 6H) 2.01-2.12 (m, 2H) 3.15-3.22 (m, 2H) 3.40-3.45 (m, 3H) 3.52 (dt, 1H) 4.19 (t, 2H) 6.26 (t, 1H) 6.44 (s, 1H) 6.54 (s, 2H) 8.00 (d, 1H) 8.58 (d, 1H)

### Example 69

### (rac)-2′-[6-amino-5-(trifluoromethyl)pyridin-3-yl]-N-[(pyridin-4-yl)methyl]-5′,6′-dihydrospiro[pyrrolidine-3,4′-pyrrolo[1,2-b]pyrazole]-1-carboxamide

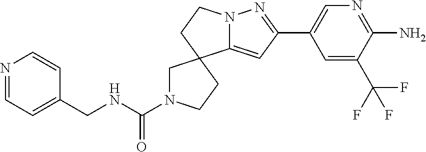 [SMILES: Nc1ncc(-c2cc3n(n2)CCC32CCN(C(=O)NCc3ccncc3)C2)cc1C(F)(F)F]

Example 69 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and 1-(pyridin-4-yl)methanamine (24.8 mg, 229 μmol).

LC-MS (Method 1): Rt=0.85 min; MS (ESIpos): m/z=458 [M+H]⁺

¹H NMR (400 MHz, DMSO-d₆) δ ppm 1.85-2.02 (m, 1H) 2.02-2.14 (m, 2H) 2.56 (t, 2H) 3.44-3.51 (m, 3H) 3.57 (s, 1H) 4.19 (t, 2H) 4.27 (d, 2H) 6.49 (s, 1H) 6.55 (s, 1H) 6.91 (t, 1H) 7.27 (d, 2H) 8.03 (d, 1H) 8.47 (d, 2H) 8.60 (d, 1H)

### Example 70

### 2′-[6-amino-5-(trifluoromethyl)pyridin-3-yl]-N-[(1R)-1-(3-cyanopyridin-2-yl)ethyl]-5′,6′-dihydrospiro[pyrrolidine-3,4′-pyrrolo[1,2-b]pyrazole]-1-carboxamide (Mixture of Stereoisomers)

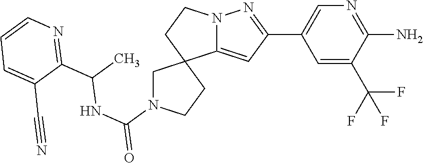 [SMILES: CC(NC(=O)N1CCC2(CCn3nc(-c4cnc(N)c(C(F)(F)F)c4)cc32)C1)c1ncccc1C#N]

Example 70 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and 2-[(1R)-1-aminoethyl]pyridine-3-carbonitrile hydrochloride (42.1 mg, 229 μmol).

LC-MS (Method 2): Rt=0.93 min; MS (ESIpos): m/z=497 [M+H]⁺

¹H NMR (400 MHz, DMSO-d₆) δ ppm 0.98-1.08 (m, 1H) 1.37-1.47 (m, 3H) 1.95-2.17 (m, 2H) 3.41-3.51 (m, 3H) 3.55 (br s, 1H) 4.11-4.26 (m, 2H) 5.05-5.15 (m, 1H) 6.46 (s, 1H) 6.50-6.58 (m, 2H) 6.66 (dd, 1H) 7.43-7.52 (m, 1H) 8.01 (d, 1H) 8.25 (d, 1H) 8.58 (t, 1H) 8.73-8.84 (m, 1H)

### Example 71

### 2′-[6-amino-5-(trifluoromethyl)pyridin-3-yl]-N-[(1S)-1-(1-methyl-1H-benzimidazol-2-yl)ethyl]-5′,6′-dihydrospiro[pyrrolidine-3,4′-pyrrolo[1,2-b]pyrazole]-1-carboxamide (Diastereomeric Mixture)

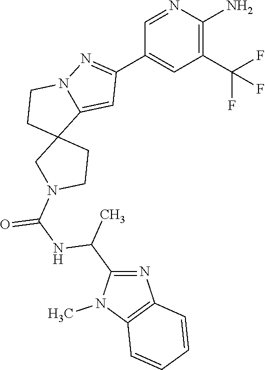 [SMILES: CC(NC(=O)N1CCC2(CCn3nc(-c4cnc(N)c(C(F)(F)F)c4)cc32)C1)c1nc2ccccc2n1C]

Example 71 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and 1-(1-methyl-1H-benzimidazol-2-yl)ethan-1-amine (54.8 mg, 313 μmol).

LC-MS (method 1): Rₜ=1.02 min; MS (ESIpos): m/z=525 [M+H]⁺

¹H-NMR (400 MHz, DMSO-d6): δ [ppm]=1.55 and 1.56 (2d, 3H), 2.00-2.12 (m, 2H), 2.51-2.55 (m, 2H), 3.39-3.50 (m, 3H), 3.51-3.61 (m, 1H), 3.77 and 3.78 (2s, 3H), 4.13-4.21 (m, 2H), 5.19-5.30 (m, 1H), 6.46 (s, 1H), 6.54 (s, 2H), 6.68 and 6.69 (2d, 1H), 7.13-7.26 (m, 2H), 7.47-7.52 (m, 1H), 7.55-7.61 (m, 1H), 8.00 (d, 1H), 8.58 (d, 1H).

### Example 72

### 2′-[6-amino-5-(trifluoromethyl)pyridin-3-yl]-N-[1-(4-fluorophenyl)-2-methoxyethyl]-5′,6′-dihydrospiro[pyrrolidine-3,4′-pyrrolo[1,2-b]pyrazole]-1-carboxamide (Mixture of Isomers)

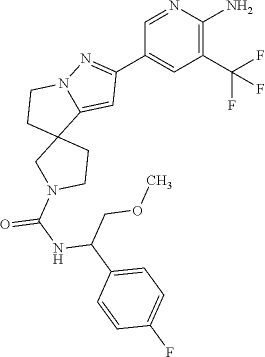 [SMILES: COCC(NC(=O)N1CCC2(CCn3nc(-c4cnc(N)c(C(F)(F)F)c4)cc32)C1)c1ccc(F)cc1]

Example 72 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (70.0 mg, 195 μmol) and 1-(4-fluorophenyl)-2-methoxyethan-1-amine (39.5 mg, 233 μmol).

LC-MS (method 1): Rt=1.04 min; MS (ESIpos): m/z=519 [M+H]⁺

1H-NMR (400 MHz, DMSO-d6): δ [ppm]=2.01-2.13 (m, 2H), 2.52-2.57 (m, 2H), 3.24 and 3.25 (2s, 3H), 3.40-3.50 (m, 4H), 3.51-3.62 (m, 2H), 4.19 (t, 2H), 4.91-5.00 (m, 1H), 6.43-6.51 (m, 2H), 6.54 (s, 2H), 7.07-7.16 (m, 2H), 7.34-7.43 (m, 2H), 8.00 and 8.00 (2d, 1H), 8.58 (d, 1H).

### Example 73

### (rac)-2′-[6-amino-5-(trifluoromethyl)pyridin-3-yl]-N-[(pyridin-2-yl)methyl]-5′,6′-dihydrospiro[pyrrolidine-3,4′-pyrrolo[1,2-b]pyrazole]-1-carboxamide

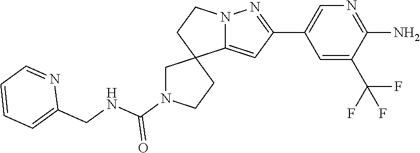 [SMILES: Nc1ncc(-c2cc3n(n2)CCC32CCN(C(=O)NCc3ccccn3)C2)cc1C(F)(F)F]

Example 73 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and 1-(pyridin-2-yl)methanamine (24.8 mg, 229 μmol).

LC-MS (Method 1): Rt=0.88 min; MS (ESIneg): m/z=456 [M−H]⁻

¹H NMR (400 MHz, DMSO-d₆) δ ppm 2.03-2.18 (m, 2H) 2.54-2.59 (m, 2H) 3.49 (s, 3H) 3.56-3.64 (m, 1H) 4.20 (t, 2H) 4.34 (d, 2H) 6.50 (s, 1H) 6.55 (s, 2H) 6.88 (t, 1H) 7.23 (dd, 1H) 7.32 (d, 1H) 7.73 (td, 1H) 8.02 (d, 1H) 8.47 (d, 1H) 8.60 (d, 1H)

### Example 74

### 2′-[6-amino-5-(trifluoromethyl)pyridin-3-yl]-N-[(1S)-1-(1-ethyl-1H-imidazol-2-yl)ethyl]-5′,6′-dihydrospiro[pyrrolidine-3,4′-pyrrolo[1,2-b]pyrazole]-1-carboxamide (Diastereomeric Mixture)

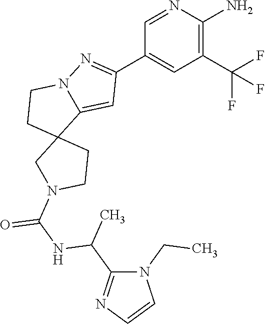 [SMILES: CCn1ccnc1C(C)NC(=O)N1CCC2(CCn3nc(-c4cnc(N)c(C(F)(F)F)c4)cc32)C1]

Example 74 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and 1-(1-ethyl-1H-imidazol-2-yl)ethan-1-amine hydrochloride (66.3 mg, 313 μmol).

¹H-NMR (400 MHz, DMSO-d6): δ [ppm]=1.25 and 1.27 (2t, 3H), 1.43 and 1.444 (2d, 3H), 1.99-2.11 (m, 2H), 3.36-3.60 (m, 4H), 3.86-4.05 (m, 2H), 4.17 (t, 2H), 4.99-5.08 (m, 1H), 6.41-6.49 (m, 2H), 6.54 (s, 2H), 6.78 and 6.78 (2d, 1H), 7.09 and 7.09 (2d, 1H), 7.98-8.02 (m, 1H), 8.56-8.59 (m, 1H).

LC-MS (method 1): Rₜ=0.92 min; MS (ESIpos): m/z=489 [M+H]⁺

### Example 75

### 2′-[6-amino-5-(trifluoromethyl)pyridin-3-yl]-N-[(1S)-1-(1-methyl-1H-imidazol-2-yl)ethyl]-5′,6′-dihydrospiro[pyrrolidine-3,4′-pyrrolo[1,2-b]pyrazole]-1-carboxamide (Diastereomeric Mixture)

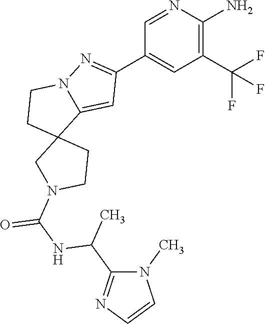 [SMILES: CC(NC(=O)N1CCC2(CCn3nc(-c4cnc(N)c(C(F)(F)F)c4)cc32)C1)c1nccn1C]

Example 75 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and 1-(1-methyl-1H-imidazol-2-yl)ethan-1-amine hydrochloride (61.9 mg, 313 μmol).

¹H-NMR (400 MHz, DMSO-d6): δ [ppm]=1.42 and 1.43 (2d, 3H), 1.99-2.11 (m, 2H), 3.36-3.56 (m, 4H), 3.58 and 3.60 (2s, 3H), 4.17 (t, 2H), 4.97-5.06 (m, 1H), 6.39-6.46 (m, 2H), 6.54 (s, 2H), 6.76 and 6.76 (2d, 1H), 7.02 and 7.02 (2d, 1H), 8.01 (d, 1H), 8.58 (d, 1H).

LC-MS (method 1): Rₜ=0.87 min; MS (ESIpos): m/z=475 [M+H]⁺

### Example 76

### (rac)-2′-[6-amino-5-(trifluoromethyl)pyridin-3-yl]-N-[(pyrazin-2-yl)methyl]-5′,6′-dihydrospiro[pyrrolidine-3,4′-pyrrolo[1,2-b]pyrazole]-1-carboxamide

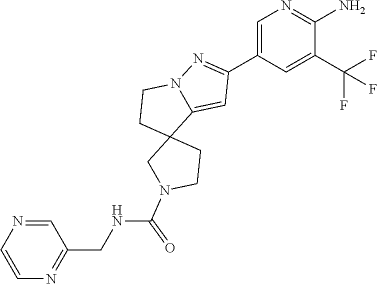 [SMILES: Nc1ncc(-c2cc3n(n2)CCC32CCN(C(=O)NCc3cnccn3)C2)cc1C(F)(F)F]

Example 76 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (100 mg, 90% purity, 250 μmol) and 1-(pyrazin-2-yl)methanamine (32.8 mg, 300 μmol).

¹H NMR (400 MHz, DMSO-d₆) δ ppm 2.04-2.16 (m, 2H) 2.52-2.58 (m, 2H) 3.44-3.52 (m, 3H) 3.53-3.63 (m, 1H) 4.19 (t, 2H) 4.39 (d, 2H) 6.49 (s, 1H) 6.54 (s, 2H) 6.98 (t, 1H) 8.02 (d, 1H) 8.50 (d, 1H) 8.56 (dd, 1H) 8.60 (dt, 2H)

LC-MS (method 1): Rₜ=0.83 min; MS (ESIneg): m/z=457 [M−H]⁻

### Example 77

### 2′-[6-amino-5-(trifluoromethyl)pyridin-3-yl]-N-[(1R)-2-methoxy-1-(pyridin-3-yl)ethyl]-5′,6′-dihydrospiro[pyrrolidine-3,4′-pyrrolo[1,2-b]pyrazole]-1-carboxamide (Mixture of Stereoisomers)

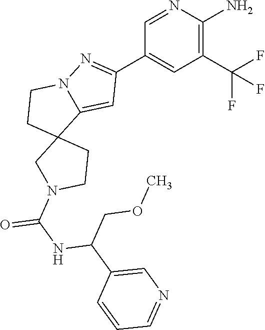 [SMILES: COCC(NC(=O)N1CCC2(CCn3nc(-c4cnc(N)c(C(F)(F)F)c4)cc32)C1)c1cccnc1]

Example 77 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (70.0 mg, 195 μmol) and (1R)-2-methoxy-1-(pyridin-3-yl)ethan-1-amine hydrochloride (52.6 mg, 233 μmol).

LC-MS (method 1): Rt=0.86 min; MS (ESIpos): m/z=502 [M+H]⁺

1H-NMR (400 MHz, DMSO-d6): δ [ppm]=2.01-2.14 (m, 2H), 2.52-2.57 (m, 2H), 3.25 and 3.26 (2s, 3H), 3.40-3.66 (m, 6H), 4.19 (t, 2H), 4.98 (q, 1H), 6.43-6.48 (m, 1H), 6.51-6.60 (m, 3H), 7.33 (td, 1H), 7.73-7.81 (m, 1H), 8.01 (t, 1H), 8.43 (td, 1H), 8.54-8.60 (m, 2H).

### Example 78

### (rac)-2′-[6-amino-5-(trifluoromethyl)pyridin-3-yl]-N-[(pyridazin-4-yl)methyl]-5′,6′-dihydrospiro[pyrrolidine-3,4′-pyrrolo[1,2-b]pyrazole]-1-carboxamide

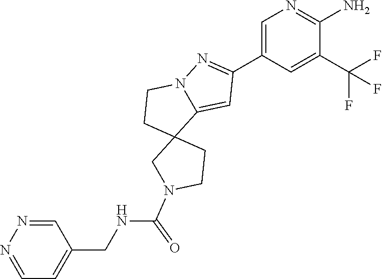 [SMILES: Nc1ncc(-c2cc3n(n2)CCC32CCN(C(=O)NCc3ccnnc3)C2)cc1C(F)(F)F]

Example 78 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (100 mg, 90% purity, 250 μmol) and 1-(pyridazin-4-yl)methanamine (32.8 mg, 300 μmol).

¹H NMR (400 MHz, DMSO-d₆) δ ppm 2.04-2.16 (m, 2H) 2.53-2.59 (m, 2H) 3.44-3.53 (m, 3H) 3.54-3.63 (m, 1H) 4.19 (t, 2H) 4.30 (d, 2H) 6.50 (s, 1H) 6.55 (s, 2H) 6.97 (t, 1H) 7.54 (dd, 1H) 8.03 (d, 1H) 8.60 (d, 1H) 9.12 (dd, 1H) 9.16 (dd, 1H)

LC-MS (method 1): Rₜ=0.79 min; MS (ESIpos): m/z=459 [M+H]⁺

### Example 79

### (rac)-2′-[6-amino-5-(trifluoromethyl)pyridin-3-yl]-N-[(1-methylpiperidin-4-yl)methyl]-5′,6′-dihydrospiro[pyrrolidine-3,4′-pyrrolo[1,2-b]pyrazole]-1-carboxamide

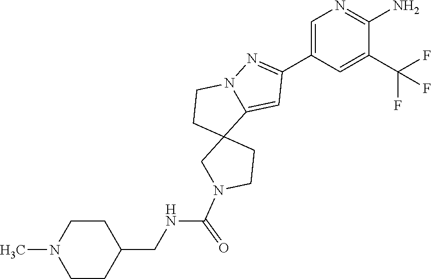 [SMILES: CN1CCC(CNC(=O)N2CCC3(CCn4nc(-c5cnc(N)c(C(F)(F)F)c5)cc43)C2)CC1]

Example 79 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (100 mg, 90% purity, 250 μmol) and 1-(1-methylpiperidin-4-yl)methanamine (38.5 mg, 300 μmol).

¹H NMR (400 MHz, DMSO-d₆) δ ppm 1.02-1.14 (m, 2H) 1.35 (ddd, 1H) 1.58 (br d, 2H) 1.75 (br t, 2H) 2.05 (br d, 2H) 2.11 (s, 3H) 2.52-2.58 (m, 2H) 2.66-2.75 (m, 2H) 2.90 (t, 2H) 3.37-3.43 (m, 3H) 3.51 (dt, 1H) 4.18 (t, 2H) 6.16 (t, 1H) 6.44 (s, 1H) 6.54 (s, 2H) 8.00 (d, 1H) 8.58 (d, 1H)

LC-MS (method 1): Rₜ=0.93 min; MS (ESIpos): m/z=479 [M+H]⁺

### Example 80

### (rac)-2′-[6-amino-5-(trifluoromethyl)pyridin-3-yl]-N-[3-(methylsulfamoyl)pyridin-2-yl]-5′,6′-dihydrospiro[pyrrolidine-3,4′-pyrrolo[1,2-b]pyrazole]-1-carboxamide

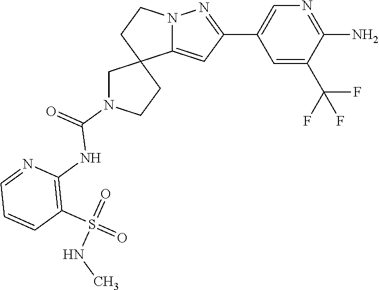 [SMILES: CNS(=O)(=O)c1cccnc1NC(=O)N1CCC2(CCn3nc(-c4cnc(N)c(C(F)(F)F)c4)cc32)C1]

Example 80 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and 2-amino-N-methylpyridine-3-sulfonamide (42.9 mg, 229 μmol).

LC-MS (method 2): Rt=0.86 min; MS (ESIneg): m/z=535 [M−H]⁻

¹H NMR (400 MHz, DMSO-d₆) δ ppm 2.16 (br t, 2H) 2.33-2.34 (m, 1H) 2.52-2.64 (m, 4H) 3.54-3.63 (m, 3H) 3.67 (br d, 1H) 4.21 (t, 2H) 6.56 (d, 3H) 7.27 (dd, 1H) 7.79 (br d, 1H) 8.02 (d, 1H) 8.12 (dd, 1H) 8.55 (br d, 1H) 8.60 (d, 1H) 8.72 (br d, 1H)

### Example 81

### (rac)-2′-[6-amino-5-(trifluoromethyl)pyridin-3-yl]-N-[(1-methyl-2-oxabicyclo[2.1.1]hexan-4-yl)methyl]-5′,6′-dihydrospiro[pyrrolidine-3,4′-pyrrolo[1,2-b]pyrazole]-1-carboxamide

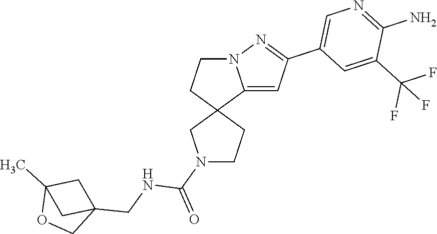 [SMILES: CC12CC(CNC(=O)N3CCC4(CCn5nc(-c6cnc(N)c(C(F)(F)F)c6)cc54)C3)(CO1)C2]

Example 81 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and 1-(1-methyl-2-oxabicyclo[2.1.1]hexan-4-yl)methanamine (31.8 mg, 250 μmol).

¹H NMR (400 MHz, DMSO-d₆) δ ppm 1.15 (d, 1H) 1.29 (s, 3H) 1.34 (dd, 2H) 1.49 (t, 2H) 2.08 (s, 2H) 2.52-2.57 (m, 2H) 3.35 (br s, 1H) 3.42 (s, 3H) 3.46 (s, 2H) 3.48-3.56 (m, 1H) 4.18 (t, 2H) 6.26 (t, 1H) 6.44 (s, 1H) 6.54 (s, 2H) 8.01 (d, 1H) 8.58 (d, 1H)

LC-MS (method 1): Rₜ=0.90 min; MS (ESIneg): m/z=475 [M−H]⁻

### Example 82

### (rac)-2′-[6-amino-5-(trifluoromethyl)pyridin-3-yl]-N-[(1-methyl-2-oxopiperidin-4-yl)methyl]-5′,6′-dihydrospiro[pyrrolidine-3,4′-pyrrolo[1,2-b]pyrazole]-1-carboxamide

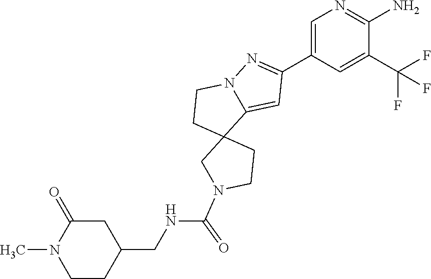 [SMILES: CN1CCC(CNC(=O)N2CCC3(CCn4nc(-c5cnc(N)c(C(F)(F)F)c5)cc43)C2)CC1=O]

Example 82 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (100 mg, 90% purity, 250 μmol) and 4-(aminomethyl)-1-methylpiperidin-2-one (42.7 mg, 300 μmol).

¹H NMR (400 MHz, DMSO-d₆) δ ppm 1.33-1.44 (m, 1H) 1.79-1.97 (m, 3H) 2.01-2.12 (m, 2H) 2.19-2.29 (m, 1H) 2.52-2.56 (m, 2H) 2.78 (s, 3H) 2.92-3.02 (m, 2H) 3.17-3.31 (m, 2H) 3.42 (s, 3H) 3.47-3.58 (m, 1H) 4.18 (t, 2H) 6.26 (t, 1H) 6.47 (s, 1H) 6.54 (s, 2H) 8.02 (d, 1H) 8.59 (d, 1H)

LC-MS (method 1): Rₜ=0.83 min; MS (ESIneg): m/z=490 [M−H]⁻

### Example 83

### (rac)-2′-[6-amino-5-(trifluoromethyl)pyridin-3-yl]-N-(2-cyclopropylpropan-2-yl)-5′,6′-dihydrospiro[pyrrolidine-3,4′-pyrrolo[1,2-b]pyrazole]-1-carboxamide

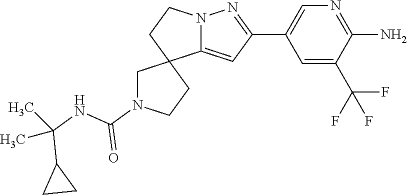 [SMILES: CC(C)(NC(=O)N1CCC2(CCn3nc(-c4cnc(N)c(C(F)(F)F)c4)cc32)C1)C1CC1]

Example 83 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and 2-cyclopropylpropan-2-amine hydrochloride (31.1 mg, 229 μmol).

LC-MS (Method 1): Rt=1.12 min; MS (ESIpos): m/z=449 [M+H]⁺

¹H NMR (400 MHz, DMSO-d₆) δ ppm 0.25-0.31 (m, 4H) 1.15 (s, 6H) 1.28-1.44 (m, 1H) 1.96-2.13 (m, 2H) 2.52-2.55 (m, 2H) 3.38-3.44 (m, 3H) 3.46-3.59 (m, 1H) 4.18 (t, 2H) 5.15 (s, 1H) 6.47 (s, 1H) 6.54 (s, 2H) 8.01 (d, 1H) 8.58 (d, 1H)

### Example 84

### 2′-[6-amino-5-(trifluoromethyl)pyridin-3-yl]-N-[(1R)-1-(4-cyanophenyl)ethyl]-5′,6′-dihydrospiro[pyrrolidine-3,4′-pyrrolo[1,2-b]pyrazole]-1-carboxamide (Mixture of Stereoisomers)

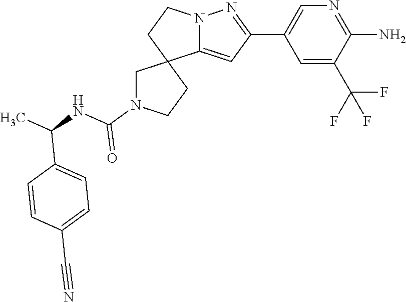 [SMILES: C[C@@H](NC(=O)N1CCC2(CCn3nc(-c4cnc(N)c(C(F)(F)F)c4)cc32)C1)c1ccc(C#N)cc1]

Example 84 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and 4-[(1R)-1-aminoethyl]benzonitrile (33.5 mg, 229 μmol).

LC-MS (Method 2): Rt=0.98 min; MS (ESIpos): m/z=496 [M+H]⁺

### Example 85

### 2′-[6-amino-5-(trifluoromethyl)pyridin-3-yl]-N-[1-methyl-2,3-dihydro-1H-inden-1-yl]-5′,6′-dihydrospiro[pyrrolidine-3,4′-pyrrolo[1,2-b]pyrazole]-1-carboxamide

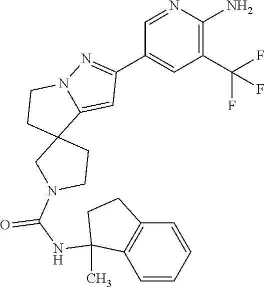 [SMILES: CC1(NC(=O)N2CCC3(CCn4nc(-c5cnc(N)c(C(F)(F)F)c5)cc43)C2)CCc2ccccc21]

Example 85 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and 1-methyl-2,3-dihydro-1H-inden-1-amine hydrochloride (45.9 mg, 250 μmol).

1H-NMR (400 MHz, DMSO-d6): δ [ppm]=1.48 (d, 3H), 1.95-2.13 (m, 3H), 2.54-2.63 (m, 2H), 2.74-2.84 (m, 1H), 2.87-2.97 (m, 1H), 3.38-3.46 (m, 3H), 3.48-3.58 (m, 1H), 4.17 (br t, 2H), 5.81 and 5.84 (2s, 1H), 6.43 and 6.46 (2s, 1H), 6.55 (s, 2H), 7.08-7.20 (m, 3H), 7.28-7.37 (m, 1H), 7.99 and 8.00 (2d, 1H), 8.58 (br s, 1H).

LC-MS (Method 1): Rₜ=1.17 min; MS (ESIPos): m/z=497 [M+H]⁺

### Example 86

### 2′-[6-amino-5-(trifluoromethyl)pyridin-3-yl]-N-[cyclopropyl(phenyl)methyl]-5′,6′-dihydrospiro[pyrrolidine-3,4′-pyrrolo[1,2-b]pyrazole]-1-carboxamide (Mixture of Isomers)

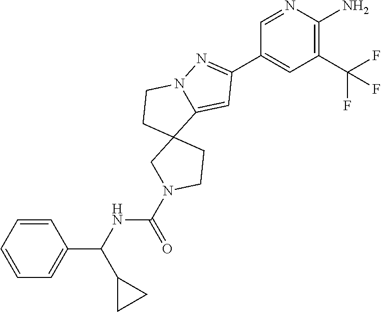 [SMILES: Nc1ncc(-c2cc3n(n2)CCC32CCN(C(=O)NC(c3ccccc3)C3CC3)C2)cc1C(F)(F)F]

Example 86 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (100 mg, 278 μmol) and (RS)-1-cyclopropyl-1-phenylmethanamine (49.1 mg, 334 μmol).

¹H-NMR (400 MHz, DMSO-d₆): δ [ppm]=0.28-0.41 (m, 2H), 0.42-0.54 (m, 2H), 1.20 (dt, 1H), 2.07 (br s, 2H), 2.53-2.59 (m, 2H), 3.40-3.51 (m, 3H), 3.52-3.62 (m, 1H), 4.06 (t, 1H), 4.19 (t, 2H), 6.43 (s, 1H), 6.55 (s, 2H), 6.65 (br d, 1H), 7.15-7.22 (m, 1H), 7.24-7.33 (m, 2H), 7.36-7.46 (m, 2H), 8.00 (s, 1H), 8.58 (s, 1H).

LC-MS (method 1): Rₜ=1.14 min; MS (ESIneg): m/z=495 [M−H]⁻

### Example 87

### 2′-[6-amino-5-(trifluoromethyl)pyridin-3-yl]-N-[−2-methyl-1-phenylpropyl]-5′,6′-dihydrospiro[pyrrolidine-3,4′-pyrrolo[1,2-b]pyrazole]-1-carboxamide (Mixture of Isomers)

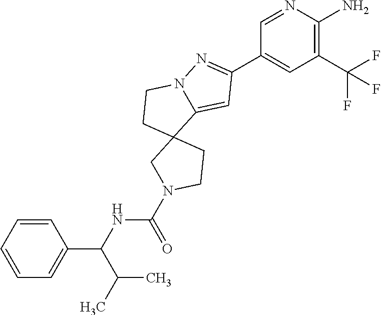 [SMILES: CC(C)C(NC(=O)N1CCC2(CCn3nc(-c4cnc(N)c(C(F)(F)F)c4)cc32)C1)c1ccccc1]

Example 87 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (100 mg, 278 μmol) and 2-methyl-1-phenylpropan-1-amine (49.8 mg, 334 μmol).

¹H-NMR (400 MHz, DMSO-d₆): δ [ppm]=0.66 (dd, 3H), 0.97 (dd, 3H), 1.94-2.11 (m, 3H), 2.52-2.55 (m, 2H), 3.37-3.63 (m, 4H), 4.18 (t, 2H), 4.34 (td, 1H), 6.33 (dd, 1H), 6.37-6.44 (m, 1H), 6.55 (s, 2H), 7.14-7.22 (m, 1H), 7.23-7.37 (m, 4H), 7.96-8.00 (m, 1H), 8.54-8.58 (m, 1H).

LC-MS (method 1): Rₜ=1.19 min; MS (ESIpos): m/z=499 [M+H]⁺

### Example 88

### 2′-[6-amino-5-(trifluoromethyl)pyridin-3-yl]-N-[(1S)-1-(pyridin-4-yl)ethyl]-5′,6′-dihydrospiro[pyrrolidine-3,4′-pyrrolo[1,2-b]pyrazole]-1-carboxamide (Mixture of Stereoisomers)

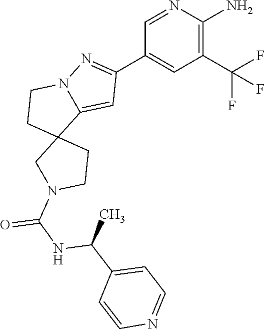 [SMILES: C[C@H](NC(=O)N1CCC2(CCn3nc(-c4cnc(N)c(C(F)(F)F)c4)cc32)C1)c1ccncc1]

Example 88 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (70.0 mg, 195 μmol) and (1S)-1-(pyridin-4-yl)ethan-1-amine (28.5 mg, 233 μmol).

¹H-NMR (400 MHz, DMSO-d6): δ [ppm]=1.36 and 1.37 (2d, 3H), 2.01-2.15 (m, 2H), 2.53-2.58 (m, 2H), 3.41-3.64 (m, 4H), 4.19 (br t, 2H), 4.77-4.86 (m, 1H), 6.48 (s, 1H), 6.55 (s, 2H), 6.60 (br dd, 1H), 7.29-7.37 (m, 2H), 8.02 (d, 1H), 8.43-8.50 (m, 2H), 8.59 (s, 1H).

LC-MS (Method 1): Rₜ=0.90 min; MS (ESIpos): m/z=472 [M+H]⁺

### Example 89

### 2′-[6-amino-5-(trifluoromethyl)pyridin-3-yl]-N-[2,2,2-trifluoro-1-phenylethyl]-5′,6′-dihydrospiro[pyrrolidine-3,4′-pyrrolo[1,2-b]pyrazole]-1-carboxamide (Mixture of Isomers)

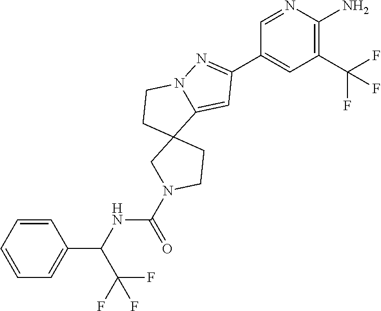 [SMILES: Nc1ncc(-c2cc3n(n2)CCC32CCN(C(=O)NC(c3ccccc3)C(F)(F)F)C2)cc1C(F)(F)F]

Example 89 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (100 mg, 278 μmol) and 2,2,2-trifluoro-1-phenylethan-1-amine hydrochloride (70.6 mg, 334 μmol).

1H-NMR (400 MHz, DMSO-d6): δ [ppm]=2.09 (br s, 2H), 2.53-2.59 (m, 2H), 3.46-3.69 (m, 4H), 4.19 (t, 2H), 5.71 (quin, 1H), 6.42-6.50 (m, 1H), 6.54 (s, 2H), 7.15 (br d, 1H), 7.34-7.50 (m, 3H), 7.57-7.65 (m, 2H), 8.00 (br s, 1H), 8.57 (s, 1H).

LC-MS (method 1): Rₜ=1.16 min; MS (ESIpos): m/z=525 [M+H]⁺

### Example 90

### 2′-[6-amino-5-(trifluoromethyl)pyridin-3-yl]-N-[1-(pyrazin-2-yl)ethyl]-5′,6′-dihydrospiro[pyrrolidine-3,4′-pyrrolo[1,2-b]pyrazole]-1-carboxamide (Mixture of Isomers)

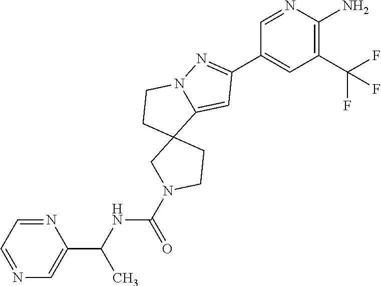 [SMILES: CC(NC(=O)N1CCC2(CCn3nc(-c4cnc(N)c(C(F)(F)F)c4)cc32)C1)c1cnccn1]

Example 90 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (100 mg, 278 μmol) and 1-(pyrazin-2-yl)ethan-1-amine (41.1 mg, 334 μmol).

¹H-NMR (400 MHz, DMSO-d₆): δ [ppm]=1.43 (dd, 3H), 2.03-2.11 (m, 2H), 2.52-2.58 (m, 2H), 3.44-3.53 (m, 3H), 3.53-3.64 (m, 1H), 4.19 (t, 2H), 4.89-4.97 (m, 1H), 6.47 (s, 1H), 6.55 (s, 2H), 6.62 (dd, 1H), 8.02 (d, 1H), 8.49 (dd, 1H), 8.56 (ddd, 1H), 8.59 (d, 1H), 8.67 (dd, 1H).

LC-MS (method 1): Rₜ=0.88 min; MS (ESIneg): m/z=471 [M−H]⁻

### Example 91

### 2′-[6-amino-5-(trifluoromethyl)pyridin-3-yl]-N-[(1R)-2-methoxy-1-phenylethyl]-5′,6′-dihydrospiro[pyrrolidine-3,4′-pyrrolo[1,2-b]pyrazole]-1-carboxamide (Mixture of Stereoisomers)

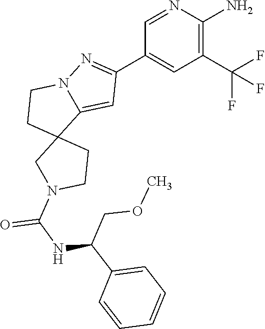 [SMILES: COC[C@H](NC(=O)N1CCC2(CCn3nc(-c4cnc(N)c(C(F)(F)F)c4)cc32)C1)c1ccccc1]

Example 91 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (70.0 mg, 195 μmol) and (1R)-2-methoxy-1-phenylethan-1-amine (35.3 mg, 233 μmol).

1H-NMR (400 MHz, DMSO-d6): δ [ppm]=2.01-2.15 (m, 2H), 2.52-2.57 (m, 2H), 3.24 and 3.26 (2s, 3H), 3.41-3.62 (m, 6H), 4.19 (t, 2H), 4.92-5.00 (m, 1H), 6.42-6.49 (m, 2H), 6.55 (s, 2H), 7.17-7.25 (m, 1H), 7.26-7.40 (m, 4H), 8.00 (s, 1H), 8.58 (s, 1H).

LC-MS (Method 1): Rₜ=1.03 min; MS (ESIpos): m/z=501 [M+H]⁺

### Example 92

### (rac)-2′-[6-amino-5-(trifluoromethyl)pyridin-3-yl]-N-[(pyridazin-3-yl)methyl]-5′,6′-dihydrospiro[pyrrolidine-3,4′-pyrrolo[1,2-b]pyrazole]-1-carboxamide

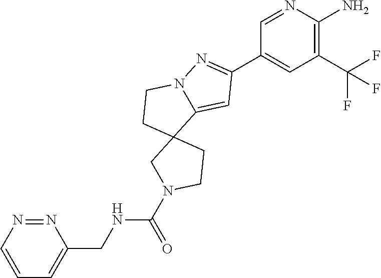 [SMILES: Nc1ncc(-c2cc3n(n2)CCC32CCN(C(=O)NCc3cccnn3)C2)cc1C(F)(F)F]

Example 92 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (100 mg, 278 μmol) and 1-(pyridazin-3-yl)methanamine (36.4 mg, 334 μmol).

¹H-NMR (400 MHz, DMSO-d₆): δ [ppm]=2.04-2.15 (m, 2H), 2.56 (t, 2H), 3.45-3.52 (m, 3H), 3.57 (s, 1H), 4.19 (t, 2H), 4.54 (d, 2H), 6.48-6.52 (m, 1H), 6.54 (s, 2H), 7.02 (t, 1H), 7.59-7.67 (m, 2H), 8.03 (d, 1H), 8.60 (d, 1H), 9.11 (dd, 1H).

LC-MS (method 1): Rₜ=0.80 min; MS (ESIpos): m/z=459 [M+H]⁺

### Example 93

### (rac)-2′-[6-amino-5-(trifluoromethyl) pyridin-3-yl]-N-[1-(2-cyanophenyl)cyclobutyl]-5′,6′-dihydrospiro[pyrrolidine-3,4′-pyrrolo[1,2-b]pyrazole]-1-carboxamide

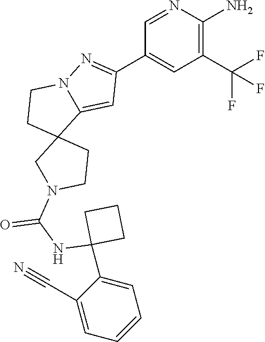 [SMILES: N#Cc1ccccc1C1(NC(=O)N2CCC3(CCn4nc(-c5cnc(N)c(C(F)(F)F)c5)cc43)C2)CCC1]

Example 93 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and 2-(1-aminocyclobutyl)benzonitrile (43.1 mg, 250 μmol).

1H-NMR (400 MHz, DMSO-d6): δ [ppm]=1.90-2.02 (m, 1H), 2.06-2.25 (m, 3H), 2.27-2.36 (m, 2H), 2.55-2.65 (m, 2H), 2.77-2.89 (m, 2H), 3.50-3.70 (m, 2H), 3.74-3.91 (m, 2H), 4.15-4.26 (m, 2H), 6.50-6.54 (m, 3H), 7.41-7.52 (m, 1H), 7.60-7.77 (m, 2H), 7.89 and 7.92 (2d, 1H), 8.03 (t, 1H), 8.58-8.62 (m, 1H), 10.13 and 10.16 (2s, 1H).

LC-MS (Method 1): Rₜ=1.27 min; MS (ESIpos): m/z=522 [M+H]⁺

### Example 94

### 2′-[6-amino-5-(trifluoromethyl)pyridin-3-yl]-N-[(1S)-2,3-dihydro-1H-inden-1-yl]-5′,6′-dihydrospiro[pyrrolidine-3,4′-pyrrolo[1,2-b]pyrazole]-1-carboxamide (Mixture of Stereoisomers)

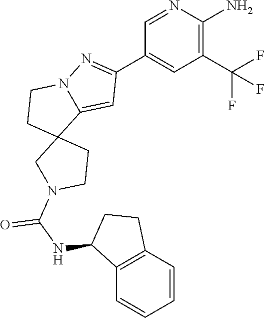 [SMILES: Nc1ncc(-c2cc3n(n2)CCC32CCN(C(=O)N[C@H]3CCc4ccccc43)C2)cc1C(F)(F)F]

Example 94 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and (1S)-2,3-dihydro-1H-inden-1-amine (44.4 mg, 75% purity, 250 μmol).

1H-NMR (400 MHz, DMSO-d6): δ [ppm]=1.81-1.91 (m, 1H), 2.02-2.14 (m, 2H), 2.31-2.42 (m, 1H), 2.52-2.58 (m, 2H), 2.71-2.82 (m, 1H), 2.86-2.94 (m, 1H), 3.41-3.52 (m, 3H), 3.53-3.64 (m, 1H), 4.19 (t, 2H), 5.17-5.27 (m, 1H), 6.42-6.50 (m, 2H), 6.55 (s, 2H), 7.11-7.28 (m, 4H), 8.02 and 8.03 (2d, 1H), 8.60 (2s, 1H).

LC-MS (Method 1): Rₜ=1.12 min; MS (ESIpos): m/z=483 [M+H]⁺

### Example 95

### 2′-[6-amino-5-(trifluoromethyl)pyridin-3-yl]-N-[(1S)-1-(4-fluorophenyl)ethyl]-5′,6′-dihydrospiro[pyrrolidine-3,4′-pyrrolo[1,2-b]pyrazole]-1-carboxamide (Mixture of Stereoisomers)

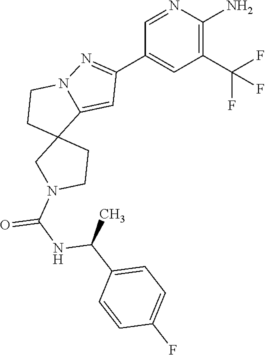 [SMILES: C[C@H](NC(=O)N1CCC2(CCn3nc(-c4cnc(N)c(C(F)(F)F)c4)cc32)C1)c1ccc(F)cc1]

Example 95 was prepared in analogy to Example 31 using (rac)-5-(5′,6′-dihydrospiro[pyrrolidine-3,4′-pyrrolo[1,2-b]pyrazol]-2′-yl)-3-(trifluoromethyl)pyridin-2-amine hydrochloride (70.0 mg, 195 μmol) and (1S)-1-(4-fluorophenyl)ethan-1-amine (32.5 mg, 233 μmol).

1H-NMR (400 MHz, DMSO-d6): δ [ppm]=1.35 and 1.36 (2d, 3H), 2.00-2.13 (m, 2H), 2.52-2.57 (m, 2H), 3.40-3.49 (m, 3H), 3.50-3.61 (m, 1H), 4.18 (t, 2H), 4.79-4.88 (m, 1H), 6.46 (s, 1H), 6.47 and 6.49 (2d, 1H), 6.54 (s, 2H), 7.05-7.15 (m, 2H), 7.32-7.41 (m, 2H), 8.01 (2s, 1H), 8.59 (s, 1H).

LC-MS (method 2): Rₜ=1.04 min; MS (ESIpos): m/z=489 [M+H]⁺

### Example 96

### 2′-[6-amino-5-(trifluoromethyl)pyridin-3-yl]-N-[(1R)-1-(2-cyanophenyl)ethyl]-5′,6′-dihydrospiro[azetidine-3,4′-pyrrolo[1,2-b]pyrazole]-1-carboxamide

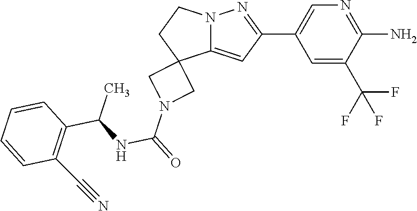 [SMILES: C[C@@H](NC(=O)N1CC2(CCn3nc(-c4cnc(N)c(C(F)(F)F)c4)cc32)C1)c1ccccc1C#N]

Example 96 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine-hydrochloride salt (75.0 mg, 217 μmol) and 2-[(1R)-1-aminoethyl]benzonitrile (34.9 mg, 239 μmol).

LC-MS (method 1): Rt=0.99 min; MS (ESIpos): m/z=482 [M+H]⁺

¹H NMR (400 MHz, DMSO-d₆) δ ppm 1.39 (d, 3H) 2.81-2.93 (m, 2H) 3.99-4.09 (m, 3H) 4.13 (t, 2H) 4.99-5.11 (m, 1H) 6.55 (s, 2H) 6.72 (s, 1H) 7.19 (d, 1H) 7.42 (td, 1H) 7.62 (d, 2H) 7.68-7.82 (m, 2H) 8.04 (d, 1H) 8.62 (d, 1H)

### Example 97

### 2′-[6-amino-5-(trifluoromethyl)pyridin-3-yl]-N-[(1R)-1-(6-cyanopyridin-3-yl)ethyl]-5′,6′-dihydrospiro[azetidine-3,4′-pyrrolo[1,2-b]pyrazole]-1-carboxamide

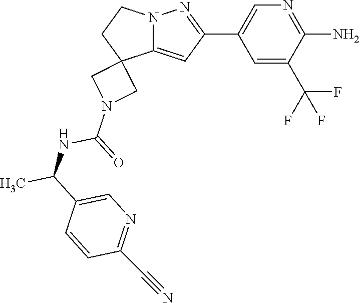 [SMILES: C[C@@H](NC(=O)N1CC2(CCn3nc(-c4cnc(N)c(C(F)(F)F)c4)cc32)C1)c1ccc(C#N)nc1]

Example 97 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine-hydrochloride salt (75.0 mg, 217 μmol) and 5-[(1R)-1-aminoethyl]pyridine-2-carbonitrile (35.1 mg, 239 μmol).

LC-MS (method 1): Rt=0.90 min; MS (ESIpos): m/z=483 [M+H]⁺

¹H NMR (400 MHz, DMSO-d₆) δ ppm 1.40 (d, 3H) 2.85 (t, 2H) 3.99-4.09 (m, 4H) 4.13 (t, 2H) 4.91 (quin, 1H) 6.55 (s, 2H) 6.73 (s, 1H) 7.08 (d, 1H) 7.94-8.07 (m, 3H) 8.62 (d, 1H) 8.74 (d, 1H)

### Example 98

### 2′-[6-amino-5-(trifluoromethyl)pyridin-3-yl]-N-[1-(pyridin-2-yl)cyclobutyl]-5′,6′-dihydrospiro[azetidine-3,4′-pyrrolo[1,2-b]pyrazole]-1-carboxamide

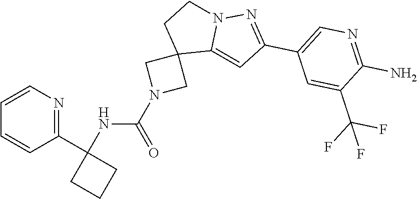 [SMILES: Nc1ncc(-c2cc3n(n2)CCC32CN(C(=O)NC3(c4ccccn4)CCC3)C2)cc1C(F)(F)F]

Example 98 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 1-(pyridin-2-yl)cyclobutan-1-amine (43.1 mg, 291 μmol).

LC-MS (method 2): Rt=0.71 min; MS (ESIpos): m/z=484 [M+H]⁺

¹H NMR (400 MHz, DMSO-d₆) δ ppm 1.81-1.96 (m, 1H) 1.96-2.09 (m, 1H) 2.37 (ddd, 2H) 2.55-2.64 (m, 2H) 2.87 (t, 2H) 4.02-4.09 (m, 4H) 4.14 (t, 2H) 6.55 (s, 2H) 6.75 (s, 1H) 7.15-7.24 (m, 2H) 7.38 (dt, 1H) 7.73 (td, 1H) 8.06 (d, 1H) 8.51-8.58 (m, 1H) 8.64 (d, 1H)

### Example 99

### 2′-[6-amino-5-(trifluoromethyl)pyridin-3-yl]-N-[(pyridin-4-yl)methyl]-5′,6′-dihydrospiro[azetidine-3,4′-pyrrolo[1,2-b]pyrazole]-1-carboxamide

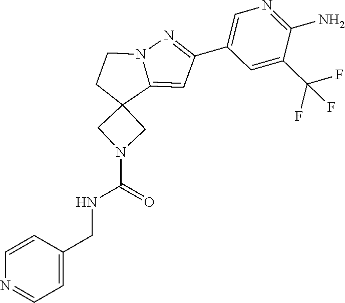 [SMILES: Nc1ncc(-c2cc3n(n2)CCC32CN(C(=O)NCc3ccncc3)C2)cc1C(F)(F)F]

Example 99 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 1-(pyridin-4-yl)methanamine (31.5 mg, 291 μmol).

LC-MS (Method 1): Rt=0.82 min; MS (ESIpos): m/z=444 [M+H]⁺

¹H NMR (400 MHz, DMSO-d₆) δ ppm 2.87 (t, 2H) 4.03-4.10 (m, 4H) 4.14 (t, 2H) 4.25 (d, 2H) 6.55 (s, 2H) 6.75 (s, 1H) 7.18 (t, 1H) 7.26-7.30 (m, 2H) 8.05 (d, 1H) 8.48-8.52 (m, 2H) 8.63 (d, 1H)

### Example 100

### (3′RS)-2-[6-amino-5-(trifluoromethyl)pyridin-3-yl]-N-[(1R)-1-(4-cyanophenyl)ethyl]-6,7-dihydrospiro[pyrazolo[5,1-c][1,4]oxazine-4,3′-pyrrolidine]-1′-carboxamide (Mixture of Stereoisomers)

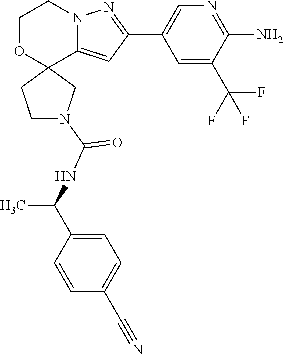 [SMILES: C[C@@H](NC(=O)N1CCC2(C1)OCCn1nc(-c3cnc(N)c(C(F)(F)F)c3)cc12)c1ccc(C#N)cc1]

Example 100 was prepared in analogy to Example 31 using 5-[6,7-dihydrospiro[pyrazolo[5,1-c][1,4]oxazine-4,3′-pyrrolidin]-2-yl]-3-(trifluoromethyl)pyridin-2-amine-hydrochloride salt (75 mg, 200 μmol) and 4-[(1R)-1-aminoethyl]benzonitrile (29.9 mg, 205 μmol).

LC-MS (Method 2): Rt=1.02 min; MS (ESIpos): m/z=512 [M+H]⁺

¹H NMR (400 MHz, DMSO-d₆) δ ppm 1.37 (d, 3H) 2.19-2.29 (m, 1H) 2.35 (br s, 1H) 3.38-3.54 (m, 2H) 3.55-3.68 (m, 1H) 3.75-3.89 (m, 1H) 4.04-4.20 (m, 4H) 4.88 (td, 1H) 6.58-6.70 (m, 3H) 6.76 (d, 1H) 7.53 (dd, 2H) 7.78 (dd, 2H) 8.01 (s, 1H) 8.58 (br s, 1H)

### Example 101

### 2′-[6-amino-5-(trifluoromethyl)pyridin-3-yl]-N-[(1R)-1-(3-cyanopyridin-2-yl)ethyl]-5′,6′-dihydrospiro[azetidine-3,4′-pyrrolo[1,2-b]pyrazole]-1-carboxamide

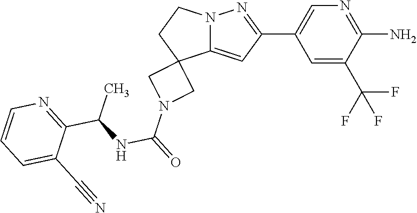 [SMILES: C[C@@H](NC(=O)N1CC2(CCn3nc(-c4cnc(N)c(C(F)(F)F)c4)cc32)C1)c1ncccc1C#N]

Example 101 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine-hydrochloride salt (75.0 mg, 217 μmol) and 2-[(1R)-1-aminoethyl]pyridine-4-carbonitrile-hydrochloride salt (43.8 mg, 239 μmol).

LC-MS (method 1): Rt=0.88 min; MS (ESIpos): m/z=483 [M+H]⁺

¹H NMR (400 MHz, DMSO-d₆) δ ppm 1.41 (d, 3H) 2.85 (t, 2H) 3.99-4.06 (m, 3H) 4.06-4.10 (m, 1H) 4.13 (t, 2H) 4.90 (quin, 1H) 6.55 (s, 2H) 6.73 (s, 1H) 7.01 (d, 1H) 8.04 (d, 1H) 8.26 (t, 1H) 8.62 (d, 1H) 8.85 (d, 1H) 8.90 (d, 1H)

### Example 102

### 2′-[6-amino-5-(trifluoromethyl)pyridin-3-yl]-N-[(1R)-1-(4-cyanophenyl)ethyl]-5′,6′-dihydrospiro[azetidine-3,4′-pyrrolo[1,2-b]pyrazole]-1-carboxamide

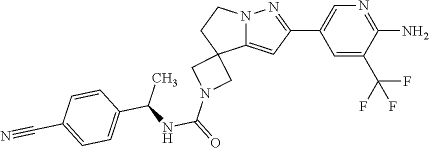 [SMILES: C[C@@H](NC(=O)N1CC2(CCn3nc(-c4cnc(N)c(C(F)(F)F)c4)cc32)C1)c1ccc(C#N)cc1]

Example 102 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine-hydrochloride salt (75.0 mg, 217 μmol) and 4-[(1R)-1-aminoethyl]benzonitrile (34.9 mg, 239 μmol).

LC-MS (Method 1): Rt=1.01 min; MS (ESIpos): m/z=482 [M+H]⁺

¹H NMR (400 MHz, DMSO-d₆) δ ppm 1.36 (d, 3H) 2.85 (t, 2H) 3.98-4.09 (m, 4H) 4.13 (t, 2H) 4.85 (quin, 1H) 6.55 (s, 2H) 6.73 (s, 1H) 7.02 (d, 1H) 7.53 (d, 2H) 7.80 (d, 2H) 8.04 (d, 1H) 8.62 (d, 1H)

### Example 103

### 2′-[6-amino-5-(trifluoromethyl)pyridin-3-yl]-N-[(1R)-1-(2-cyanopyridin-4-yl)ethyl]-5′,6′-dihydrospiro[azetidine-3,4′-pyrrolo[1,2-b]pyrazole]-1-carboxamide

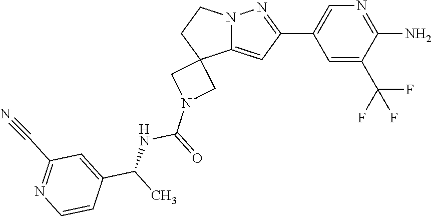 [SMILES: C[C@@H](NC(=O)N1CC2(CCn3nc(-c4cnc(N)c(C(F)(F)F)c4)cc32)C1)c1ccnc(C#N)c1]

Example 103 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine-hydrochloride salt (75.0 mg, 217 μmol) and 4-[(1R)-1-aminoethyl]pyridine-2-carbonitrile (35.1 mg, 239 μmol).

LC-MS (Method 1): Rt=0.93 min; MS (ESIpos): m/z=483 [M+H]⁺

¹H NMR (400 MHz, DMSO-d₆) δ ppm 1.38 (d, 3H) 2.86 (t, 2H) 4.01-4.07 (m, 3H) 4.07-4.18 (m, 3H) 4.85 (quin, 1H) 6.55 (s, 2H) 6.73 (s, 1H) 7.07 (d, 1H) 7.69 (dd, 1H) 7.99-8.01 (m, 1H) 8.04 (d, 1H) 8.62 (d, 1H) 8.70 (d, 1H)

### Example 104

### 2′-[6-amino-5-(trifluoromethyl)pyridin-3-yl]-N-[(1R)-1-(2-chloro-4-cyanophenyl)ethyl]-5′,6′-dihydrospiro[azetidine-3,4′-pyrrolo[1,2-b]pyrazole]-1-carboxamide

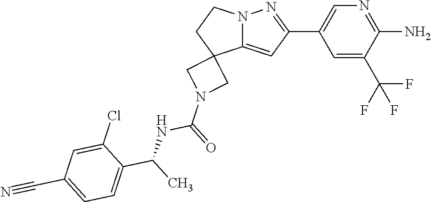 [SMILES: C[C@@H](NC(=O)N1CC2(CCn3nc(-c4cnc(N)c(C(F)(F)F)c4)cc32)C1)c1ccc(C#N)cc1Cl]

Example 104 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine-hydrochloride salt (75.0 mg, 217 μmol) and 4-[(1R)-1-aminoethyl]-3-chlorobenzonitrile (43.1 mg, 239 μmol).

LC-MS (method 1): Rt=1.06 min; MS (ESIpos): m/z=516 [M+H]⁺

¹H NMR (400 MHz, DMSO-d₆) δ ppm 1.33 (d, 3H) 2.86 (t, 2H) 4.00-4.06 (m, 3H) 4.06-4.10 (m, 1H) 4.13 (t, 2H) 5.11 (quin, 1H) 6.55 (s, 2H) 6.73 (s, 1H) 7.20 (d, 1H) 7.67 (d, 1H) 7.87 (dd, 1H) 8.01 (d, 1H) 8.04 (d, 1H) 8.62 (d, 1H)

### Example 105

### 2′-[6-amino-5-(trifluoromethyl)pyridin-3-yl]-N-[2-(2-fluorophenyl)propan-2-yl]-5′,6′-dihydrospiro[azetidine-3,4′-pyrrolo[1,2-b]pyrazole]-1-carboxamide

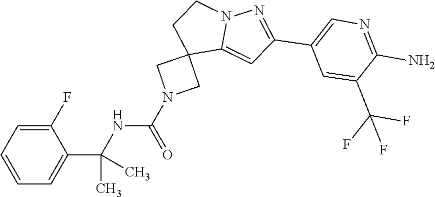 [SMILES: CC(C)(NC(=O)N1CC2(CCn3nc(-c4cnc(N)c(C(F)(F)F)c4)cc32)C1)c1ccccc1F]

Example 105 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 2-(2-fluorophenyl)propan-2-amine-hydrochloride salt (55.2 mg, 291 μmol).

### Example 106

2′-[6-amino-5-(trifluoromethyl)pyridin-3-yl]-N-[1-(1-ethyl-3,5-dimethyl-1H-pyrazol-4-yl)ethyl]-5′,6′-dihydrospiro[azetidine-3,4′-pyrrolo[1,2-b]pyrazole]-1-carboxamide

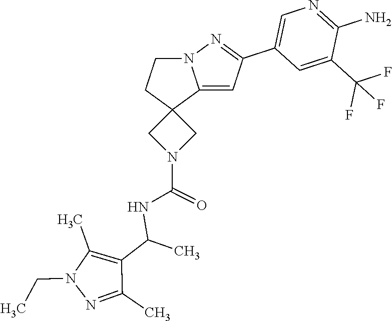 [SMILES: CCn1nc(C)c(C(C)NC(=O)N2CC3(CCn4nc(-c5cnc(N)c(C(F)(F)F)c5)cc43)C2)c1C]

Example 106 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 1-(1-ethyl-3,5-dimethyl-1H-pyrazol-4-yl)ethan-1-amine (48.7 mg, 291 μmol).

LC-MS (method 2): Rt=0.85 min; MS (ESIpos): m/z=503 [M+H]⁺

¹H NMR (400 MHz, DMSO-d₆) δ ppm 1.20-1.26 (m, 3H) 1.34 (d, 3H) 2.14 (s, 3H) 2.21 (s, 3H) 2.79-2.86 (m, 2H) 3.87-4.02 (m, 6H) 4.11 (t, 2H) 4.62-4.73 (m, 1H) 6.50-6.60 (m, 3H) 6.68-6.72 (m, 1H) 8.03 (d, 1H) 8.61 (d, 1H)

### Example 107

### 2′-[6-amino-5-(trifluoromethyl)pyridin-3-yl]-N-[2-(pyridin-2-yl)propan-2-yl]-5′,6′-dihydrospiro[azetidine-3,4′-pyrrolo[1,2-b]pyrazole]-1-carboxamide

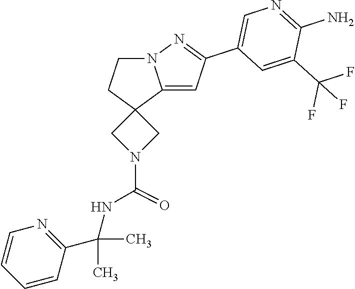 [SMILES: CC(C)(NC(=O)N1CC2(CCn3nc(-c4cnc(N)c(C(F)(F)F)c4)cc32)C1)c1ccccn1]

Example 107 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 2-(pyridin-2-yl)propan-2-amine (39.6 mg, 291 μmol).

LC-MS (Method 2): Rt=0.75 min; MS (ESIpos): m/z=472 [M+H]⁺

¹H NMR (400 MHz, DMSO-d₆) δ ppm 1.57 (s, 6H) 2.86 (t, 2H) 4.00-4.08 (m, 4H) 4.14 (t, 2H) 6.55 (s, 2H) 6.71-6.76 (m, 2H) 7.20 (ddd, 1H) 7.45 (d, 1H) 7.74 (td, 1H) 8.05 (d, 1H) 8.45-8.49 (m, 1H) 8.64 (d, 1H)

### Example 108

### 2′-[6-amino-5-(trifluoromethyl)pyridin-3-yl]-N-[1-(1-methyl-1H-pyrazol-4-yl)ethyl]-5′,6′-dihydrospiro[azetidine-3,4′-pyrrolo[1,2-b]pyrazole]-1-carboxamide

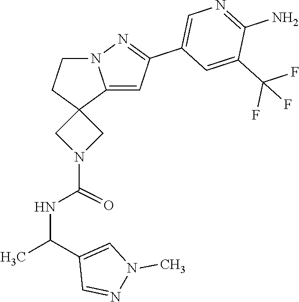 [SMILES: CC(NC(=O)N1CC2(CCn3nc(-c4cnc(N)c(C(F)(F)F)c4)cc32)C1)c1cnn(C)c1]

Example 108 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 1-(1-methyl-1H-pyrazol-4-yl)ethan-1-amine (36.4 mg, 291 μmol).

LC-MS (Method 2): Rt=0.88 min; MS (ESIpos): m/z=461 [M+H]⁺

¹H NMR (400 MHz, DMSO-d₆) δ ppm 1.33 (d, 3H) 2.84 (t, 2H) 3.74-3.82 (m, 3H) 3.95-4.04 (m, 4H) 4.12 (t, 2H) 4.70-4.81 (m, 1H) 6.54 (s, 2H) 6.63 (d, 1H) 6.72 (s, 1H) 7.31 (s, 1H) 7.53 (s, 1H) 8.04 (d, 1H) 8.62 (d, 1H)

### Example 109

### 2′-[6-amino-5-(trifluoromethyl)pyridin-3-yl]-N-[1-(1,3,4-trimethyl-1H-pyrazol-5-yl)ethyl]-5′,6′-dihydrospiro[azetidine-3,4′-pyrrolo[1,2-b]pyrazole]-1-carboxamide

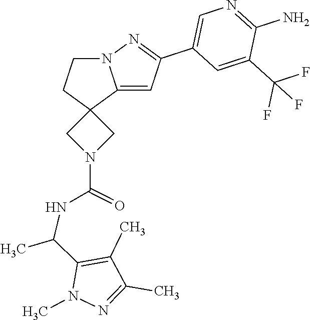 [SMILES: Cc1nn(C)c(C(C)NC(=O)N2CC3(CCn4nc(-c5cnc(N)c(C(F)(F)F)c5)cc43)C2)c1C]

Example 109 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (50.5 mg, 163 μmol) and 1-(1,3,4-trimethyl-1H-pyrazol-5-yl)ethan-1-amine (30.0 mg, 196 μmol).

LC-MS (Method 2): Rt=0.89 min; MS (ESIpos): m/z=489 [M+H]⁺

¹H NMR (400 MHz, DMSO-d₆) δ ppm 1.40 (d, 3H) 1.97 (d, 6H) 2.83 (t, 2H) 3.71 (s, 3H) 3.93-4.05 (m, 4H) 4.12 (t, 2H) 4.88 (quin, 1H) 6.54 (s, 2H) 6.70 (s, 1H) 6.91 (d, 1H) 8.03 (d, 1H) 8.61 (d, 1H)

### Example 110

### 2′-[6-amino-5-(trifluoromethyl)pyridin-3-yl]-N-{1-[3-(difluoromethyl)-1-methyl-1H-pyrazol-4-yl]ethyl}-5′,6′-dihydrospiro[azetidine-3,4′-pyrrolo[1,2-b]pyrazole]-1-carboxamide

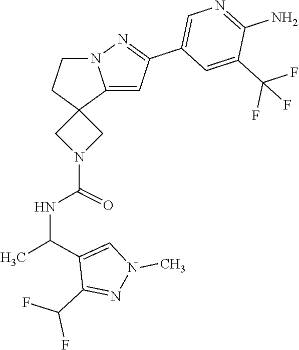 [SMILES: CC(NC(=O)N1CC2(CCn3nc(-c4cnc(N)c(C(F)(F)F)c4)cc32)C1)c1cn(C)nc1C(F)F]

Example 110 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 1-[3-(difluoromethyl)-1-methyl-1H-pyrazol-4-yl]ethan-1-amine (51.0 mg, 291 μmol).

LC-MS (Method 2): Rt=0.93 min; MS (ESIpos): m/z=511 [M+H]⁺

¹H NMR (400 MHz, DMSO-d₆) δ ppm 1.35 (d, 3H) 2.83 (t, 2H) 3.84 (s, 3H) 3.95-4.04 (m, 4H) 4.12 (t, 2H) 4.90 (quin, 1H) 6.55 (s, 2H) 6.68-6.75 (m, 2H) 6.98 (s, 1H) 7.70 (s, 1H) 8.04 (d, 1H) 8.61 (d, 1H)

### Example 111

### (rac)-2-[6-amino-5-(trifluoromethyl)pyridin-3-yl]-N-[2-(pyridin-4-yl)propan-2-yl]-6,7-dihydrospiro[pyrazolo[5,1-c][1,4]oxazine-4,3′-pyrrolidine]-1′-carboxamide

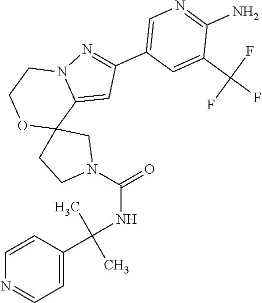 [SMILES: CC(C)(NC(=O)N1CCC2(C1)OCCn1nc(-c3cnc(N)c(C(F)(F)F)c3)cc12)c1ccncc1]

Example 111 was prepared in analogy to Example 31 using 5-[6,7-dihydrospiro[pyrazolo[5,1-c][1,4]oxazine-4,3′-pyrrolidin]-2-yl]-3-(trifluoromethyl)pyridin-2-amine-hydrochloride salt (100 mg, 266 μmol) and 2-(pyridin-4-yl)propan-2-amine (43.5 mg, 319 μmol).

¹H NMR (400 MHz, DMSO-d₆) δ ppm 1.54 (s, 6H) 2.18-2.30 (m, 1H) 2.31-2.38 (m, 1H) 3.36-3.53 (m, 2H) 3.61 (br t, 1H) 3.80 (br d, 1H) 4.07-4.18 (m, 4H) 6.25 (s, 1H) 6.60 (s, 2H) 6.77 (s, 1H) 7.30-7.33 (m, 2H) 8.02 (d, 1H) 8.41-8.48 (m, 2H) 8.54-8.60 (m, 1H)

LC-MS (Method 1): Rt=0.94 min; MS (ESIpos): m/z=502 [M+H]⁺

### Example 112

### (rac)-2-[6-amino-5-(trifluoromethyl)pyridin-3-yl]-N-[1-(pyridin-3-yl)cyclobutyl]-6,7-dihydrospiro[pyrazolo[5,1-c][1,4]oxazine-4,3′-pyrrolidine]-1′-carboxamide

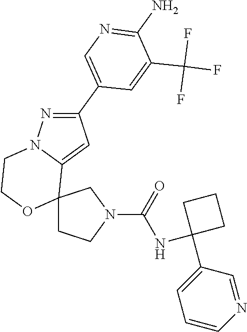 [SMILES: Nc1ncc(-c2cc3n(n2)CCOC32CCN(C(=O)NC3(c4cccnc4)CCC3)C2)cc1C(F)(F)F]

Example 112 was prepared in analogy to Example 31 using 5-[6,7-dihydrospiro[pyrazolo[5,1-c][1,4]oxazine-4,3′-pyrrolidin]-2-yl]-3-(trifluoromethyl)pyridin-2-amine-hydrochloride salt (100 mg, 266 μmol) and 1-(pyridin-3-yl)cyclobutan-1-amine (47.3 mg, 319 μmol).

LC-MS (Method 1): Rt=0.94 min; MS (ESIpos): m/z=514 [M+H]⁺

¹H NMR (400 MHz, DMSO-d₆) δ ppm 1.74-1.86 (m, 1H) 1.97-2.09 (m, 1H) 2.22 (br d, 1H) 2.30-2.37 (m, 1H) 2.38-2.48 (m, 4H) 3.36-3.43 (m, 1H) 3.47 (br d, 1H) 3.58 (br t, 1H) 3.77 (br d, 1H) 4.05-4.17 (m, 4H) 6.60 (s, 2H) 6.75 (s, 1H) 6.83 (s, 1H) 7.28-7.36 (m, 1H) 7.76-7.82 (m, 1H) 8.01 (d, 1H) 8.38 (dd, 1H) 8.57 (d, 1H) 8.64 (dd, 1H)

### Example 113

### 2′-[6-amino-5-(trifluoromethyl)pyridin-3-yl]-N-[1-(1-ethyl-5-methyl-1H-pyrazol-4-yl)ethyl]-5′,6′-dihydrospiro[azetidine-3,4′-pyrrolo[1,2-b]pyrazole]-1-carboxamide

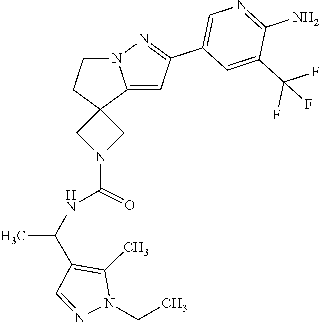 [SMILES: CCn1ncc(C(C)NC(=O)N2CC3(CCn4nc(-c5cnc(N)c(C(F)(F)F)c5)cc43)C2)c1C]

Example 113 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 1-(1-ethyl-5-methyl-1H-pyrazol-4-yl)ethan-1-amine (44.6 mg, 291 μmol).

LC-MS (Method 2): Rt=0.89 min; MS (ESIpos): m/z=489 [M+H]⁺

¹H NMR (400 MHz, DMSO-d₆) δ ppm 1.26 (t, 3H) 1.33 (d, 3H) 2.21 (s, 3H) 2.82 (t, 2H) 3.93-4.04 (m, 6H) 4.12 (t, 2H) 4.68-4.78 (m, 1H) 6.54 (s, 2H) 6.60 (d, 1H) 6.70 (s, 1H) 7.31 (s, 1H) 8.04 (d, 1H) 8.61 (d, 1H)

### Example 114

### 2′-[6-amino-5-(trifluoromethyl)pyridin-3-yl]-N-[1-(1,5-dimethyl-1H-pyrazol-4-yl)ethyl]-5′,6′-dihydrospiro[azetidine-3,4′-pyrrolo[1,2-b]pyrazole]-1-carboxamide

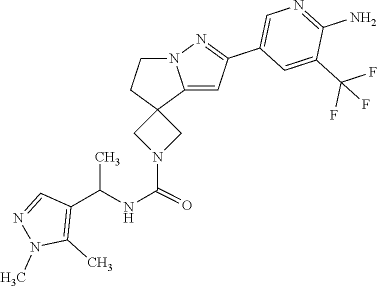 [SMILES: Cc1c(C(C)NC(=O)N2CC3(CCn4nc(-c5cnc(N)c(C(F)(F)F)c5)cc43)C2)cnn1C]

Example 114 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 1-(1,5-dimethyl-1H-pyrazol-4-yl)ethan-1-amine (40.5 mg, 291 μmol).

LC-MS (Method 2): Rt=0.85 min; MS (ESIpos): m/z=475 [M+H]⁺

¹H NMR (400 MHz, DMSO-d₆) δ ppm 1.33 (d, 3H) 2.20 (s, 3H) 2.82 (t, 2H) 3.67 (s, 3H) 3.92-4.02 (m, 4H) 4.12 (t, 2H) 4.67-4.77 (m, 1H) 6.54 (s, 2H) 6.59 (d, 1H) 6.70 (s, 1H) 7.28 (s, 1H) 8.04 (d, 1H) 8.62 (d, 1H)

### Example 115

### 2′-[6-amino-5-(trifluoromethyl)pyridin-3-yl]-N-[1-(1,3,5-trimethyl-1H-pyrazol-4-yl)ethyl]-5′,6′-dihydrospiro[azetidine-3,4′-pyrrolo[1,2-b]pyrazole]-1-carboxamide

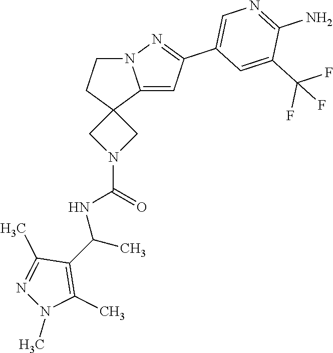 [SMILES: Cc1nn(C)c(C)c1C(C)NC(=O)N1CC2(CCn3nc(-c4cnc(N)c(C(F)(F)F)c4)cc32)C1]

Example 115 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 1-(1,3,5-trimethyl-1H-pyrazol-4-yl)ethan-1-amine (44.6 mg, 291 μmol).

LC-MS (Method 2): Rt=0.86 min; MS (ESIpos): m/z=489 [M+H]⁺

¹H NMR (500 MHz, DMSO-d₆) δ ppm 1.34 (d, 3H) 2.13 (s, 3H) 2.20 (s, 3H) 2.82 (t, 2H) 3.57 (s, 3H) 3.92-4.01 (m, 4H) 4.12 (t, 2H) 4.66 (quin, 1H) 6.53 (s, 2H) 6.58 (d, 1H) 6.68 (s, 1H) 8.03 (d, 1H) 8.61 (d, 1H)

### Example 116

### 2′-[6-amino-5-(trifluoromethyl)pyridin-3-yl]-N-[(1R)-1-(pyridin-3-yl)ethyl]-5′,6′-dihydrospiro[azetidine-3,4′-pyrrolo[1,2-b]pyrazole]-1-carboxamide

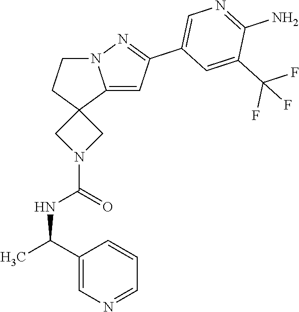 [SMILES: C[C@@H](NC(=O)N1CC2(CCn3nc(-c4cnc(N)c(C(F)(F)F)c4)cc32)C1)c1cccnc1]

Example 116 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and (1R)-1-(pyridin-3-yl)ethan-1-amine-hydrochloride salt (56.8 mg, 291 μmol).

LC-MS (Method 2): Rt=0.68 min; MS (ESIpos): m/z=458 [M+H]⁺

¹H NMR (400 MHz, DMSO-d₆) δ ppm 1.39 (d, 3H) 2.85 (t, 2H) 3.99-4.07 (m, 4H) 4.13 (t, 2H) 4.85 (t, 1H) 6.49-6.61 (m, 2H) 6.72 (s, 1H) 6.95-7.04 (m, 1H) 7.32-7.38 (m, 1H) 7.74 (dt, 1H) 8.04 (d, 1H) 8.43 (dd, 1H) 8.56 (d, 1H) 8.62 (d, 1H)

### Example 117

### 2-[6-amino-5-(trifluoromethyl)pyridin-3-yl]-N-[(1R)-1-(2-cyanopyridin-4-yl)ethyl]-6,7-dihydrospiro[pyrazolo[5,1-c][1,4]oxazine-4,3′-pyrrolidine]-1′-carboxamide (Mixture of Stereoisomers)

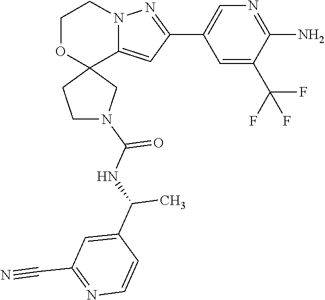 [SMILES: C[C@@H](NC(=O)N1CCC2(C1)OCCn1nc(-c3cnc(N)c(C(F)(F)F)c3)cc12)c1ccnc(C#N)c1]

Example 117 was prepared in analogy to Example 31 using 5-[6,7-dihydrospiro[pyrazolo[5,1-c][1,4]oxazine-4,3′-pyrrolidin]-2-yl]-3-(trifluoromethyl)pyridin-2-amine-hydrochloride salt (75 mg, 200 μmol) and 4-[(1R)-1-aminoethyl]pyridine-2-carbonitrile (30.1 mg, 205 μmol).

LC-MS (Method 2): Rt=0.94 min; MS (ESIpos): m/z=513 [M+H]⁺

¹H NMR (400 MHz, DMSO-d₆) δ ppm 1.38 (d, 3H) 2.19-2.32 (m, 1H) 2.34-2.41 (m, 1H) 3.38-3.71 (m, 3H) 3.76-3.89 (m, 1H) 4.01-4.18 (m, 4H) 4.80-4.93 (m, 1H) 6.60 (s, 2H) 6.66-6.74 (m, 1H) 6.76 (d, 1H) 7.69 (ddd, 1H) 7.99-8.03 (m, 2H) 8.58 (s, 1H) 8.66-8.70 (m, 1H)

### Example 118

### 2-[6-amino-5-(trifluoromethyl)pyridin-3-yl]-N-[(1R)-1-(3-cyanopyridin-2-yl)ethyl]-6,7-dihydrospiro[pyrazolo[5,1-c][1,4]oxazine-4,3′-pyrrolidine]-1′-carboxamide (Mixture of Stereoisomers)

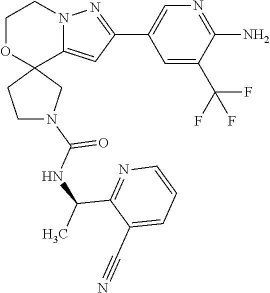 [SMILES: C[C@@H](NC(=O)N1CCC2(C1)OCCn1nc(-c3cnc(N)c(C(F)(F)F)c3)cc12)c1ncccc1C#N]

Example 118 was prepared in analogy to Example 31 using 5-[6,7-dihydrospiro[pyrazolo[5,1-c][1,4]oxazine-4,3′-pyrrolidin]-2-yl]-3-(trifluoromethyl)pyridin-2-amine-hydrochloride salt (75 mg, 200 μmol) and 2-[(1R)-1-aminoethyl]pyridine-3-carbonitrile-hydrochloride salt (37.6 mg, 205 μmol).

LC-MS (Method 2): Rt=0.94 min; MS (ESIpos): m/z=513 [M+H]⁺

¹H NMR (400 MHz, DMSO-d₆) δ ppm 1.42 (dd, 3H) 2.17-2.28 (m, 1H) 2.30-2.39 (m, 1H) 3.37-3.52 (m, 2H) 3.61 (dt, 1H) 3.81 (br dd, 1H) 4.00-4.18 (m, 4H) 5.03-5.18 (m, 1H) 6.60 (s, 2H) 6.69 (dd, 1H) 6.72-6.78 (m, 1H) 7.47 (ddd, 1H) 8.01 (d, 1H) 8.26 (dd, 1H) 8.58 (s, 1H) 8.80 (dt, 1H)

### Example 119

### 2-[6-amino-5-(trifluoromethyl)pyridin-3-yl]-N-[(1R)-1-(6-cyanopyridin-3-yl)ethyl]-6,7-dihydrospiro[pyrazolo[5,1-c][1,4]oxazine-4,3′-pyrrolidine]-1′-carboxamide (Mixture of Stereoisomers)

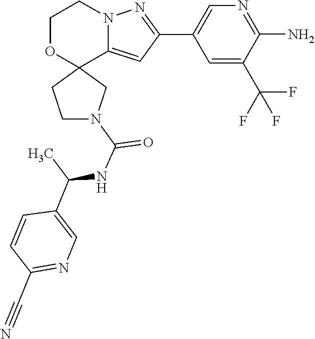 [SMILES: C[C@@H](NC(=O)N1CCC2(C1)OCCn1nc(-c3cnc(N)c(C(F)(F)F)c3)cc12)c1ccc(C#N)nc1]

Example 119 was prepared in analogy to Example 31 using 5-[6,7-dihydrospiro[pyrazolo[5,1-c][1,4]oxazine-4,3′-pyrrolidin]-2-yl]-3-(trifluoromethyl)pyridin-2-amine-hydrochloride salt (75 mg, 200 μmol) and 5-[(1R)-1-aminoethyl]pyridine-2-carbonitrile (30.1 mg, 205 μmol).

LC-MS (Method 2): Rt=0.94 min; MS (ESIpos): m/z=513 [M+H]⁺

¹H NMR (400 MHz, DMSO-d₆) δ ppm 1.41 (d, 3H) 2.17-2.29 (m, 1H) 2.31-2.41 (m, 1H) 3.37-3.67 (m, 3H) 3.75-3.87 (m, 1H) 4.04-4.18 (m, 4H) 4.88-4.97 (m, 1H) 6.60 (s, 2H) 6.66-6.74 (m, 1H) 6.76 (d, 1H) 7.95-8.04 (m, 3H) 8.58 (t, 1H) 8.74 (d, 1H)

### Example 120

### 2′-[6-amino-5-(trifluoromethyl)pyridin-3-yl]-N-[(pyridin-2-yl)methyl]-5′,6′-dihydrospiro[azetidine-3,4′-pyrrolo[1,2-b]pyrazole]-1-carboxamide

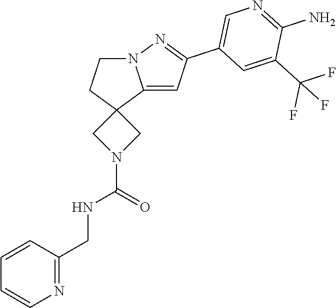 [SMILES: Nc1ncc(-c2cc3n(n2)CCC32CN(C(=O)NCc3ccccn3)C2)cc1C(F)(F)F]

Example 120 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 1-(pyridin-2-yl)methanamine (31.5 mg, 291 μmol).

LC-MS (OAo1b02): Rt=0.86 min; MS (ESIpos): m/z=444 [M+H]⁺

¹H NMR (400 MHz, DMSO-d₆) δ ppm 2.87 (t, 2H) 4.03-4.10 (m, 4H) 4.14 (t, 2H) 4.32 (d, 2H) 6.55 (s, 2H) 6.75 (s, 1H) 7.16 (t, 1H) 7.25 (ddd, 1H) 7.32 (d, 1H) 7.77 (td, 1H) 8.05 (d, 1H) 8.46-8.52 (m, 1H) 8.63 (d, 1H)

### Example 121

### (rac)-2-[6-amino-5-(trifluoromethyl)pyridin-3-yl]-N-[(1R)-1-(2-cyanophenyl)ethyl]-6,7-dihydrospiro[pyrazolo[5,1-c][1,4]oxazine-4,3′-pyrrolidine]-1′-carboxamide

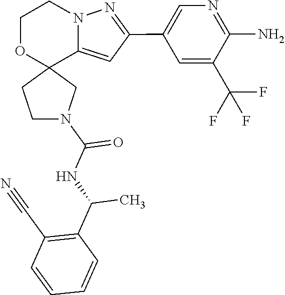 [SMILES: C[C@@H](NC(=O)N1CCC2(C1)OCCn1nc(-c3cnc(N)c(C(F)(F)F)c3)cc12)c1ccccc1C#N]

Example 121 was prepared in analogy to Example 31 using 5-[6,7-dihydrospiro[pyrazolo[5,1-c][1,4]oxazine-4,3′-pyrrolidin]-2-yl]-3-(trifluoromethyl)pyridin-2-amine-hydrochloride salt (75 mg, 200 μmol) and 2-[(1R)-1-aminoethyl]benzonitrile (29.9 mg, 205 μmol).

LC-MS (Method 2): Rt=1.01 min; MS (ESIpos): m/z=512 [M+H]⁺

¹H NMR (400 MHz, DMSO-d₆) δ ppm 1.40 (d, 3H) 2.19-2.29 (m, 1H) 2.31-2.41 (m, 1H) 3.39-3.69 (m, 3H) 3.82 (dd, 1H) 4.04-4.20 (m, 4H) 5.05 (quin, 1H) 6.60 (s, 2H) 6.73-6.84 (m, 2H) 7.40 (tt, 1H) 7.61-7.66 (m, 1H) 7.68 (ddd, 1H) 7.76 (dd, 1H) 8.01 (t, 1H) 8.56-8.60 (m, 1H)

### Example 122

### 2′-[6-amino-5-(trifluoromethyl)pyridin-3-yl]-N-[(1R)-1-(4-fluorophenyl)ethyl]-5′,6′-dihydrospiro[azetidine-3,4′-pyrrolo[1,2-b]pyrazole]-1-carboxamide

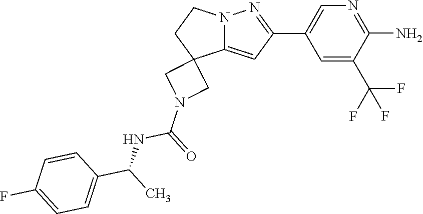 [SMILES: C[C@@H](NC(=O)N1CC2(CCn3nc(-c4cnc(N)c(C(F)(F)F)c4)cc32)C1)c1ccc(F)cc1]

Example 122 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and (1R)-1-(4-fluorophenyl)ethan-1-amine (40.5 mg, 291 μmol).

LC-MS (Method 2): Rt=1.05 min; MS (ESIpos): m/z=475 [M+H]⁺

¹H NMR (400 MHz, DMSO-d₆) δ ppm 1.35 (d, 3H) 2.84 (t, 2H) 3.97-4.07 (m, 4H) 4.13 (t, 2H) 4.81 (quin, 1H) 6.57 (br s, 2H) 6.72 (s, 1H) 6.90 (d, 1H) 7.14 (t, 2H) 7.37 (dd, 2H) 8.05 (d, 1H) 8.62 (d, 1H)

### Example 123

### 2′-[6-amino-5-(trifluoromethyl)pyridin-3-yl]-N-[2-(4-fluorophenyl)propan-2-yl]-5′,6′-dihydrospiro[azetidine-3,4′-pyrrolo[1,2-b]pyrazole]-1-carboxamide

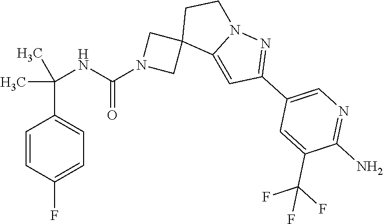 [SMILES: CC(C)(NC(=O)N1CC2(CCn3nc(-c4cnc(N)c(C(F)(F)F)c4)cc32)C1)c1ccc(F)cc1]

Example 123 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 2-(4-fluorophenyl)propan-2-amine-hydrochloride salt (55.2 mg, 291 μmol).

LC-MS (Method 2): Rt=1.11 min; MS (ESIpos): m/z=489 [M+H]⁺

¹H NMR (400 MHz, DMSO-d₆) δ ppm 1.55 (s, 6H) 2.80-2.89 (m, 2H) 3.94-4.08 (m, 4H) 4.13 (t, 2H) 6.50-6.61 (m, 3H) 6.71 (s, 1H) 7.06-7.13 (m, 2H) 7.34-7.41 (m, 2H) 8.05 (d, 1H) 8.63 (d, 1H)

### Example 124

### 2′-[6-amino-5-(trifluoromethyl)pyridin-3-yl]-N-[2-(pyridin-3-yl)propan-2-yl]-5′,6′-dihydrospiro[azetidine-3,4′-pyrrolo[1,2-b]pyrazole]-1-carboxamide

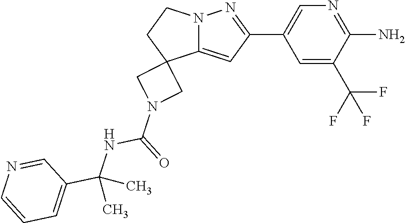 [SMILES: CC(C)(NC(=O)N1CC2(CCn3nc(-c4cnc(N)c(C(F)(F)F)c4)cc32)C1)c1cccnc1]

Example 124 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 2-(pyridin-3-yl)propan-2-amine (39.6 mg, 291 μmol).

LC-MS (Method 2): Rt=0.70 min; MS (ESIpos): m/z=472 [M+H]⁺

¹H NMR (400 MHz, DMSO-d₆) δ ppm 1.58 (s, 6H) 2.85 (t, 2H) 3.99-4.08 (m, 4H) 4.13 (t, 2H) 6.55 (s, 2H) 6.68 (s, 1H) 6.72 (s, 1H) 7.29-7.35 (m, 1H) 7.70-7.75 (m, 1H) 8.06 (d, 1H) 8.38 (dd, 1H) 8.60 (d, 1H) 8.64 (d, 1H)

### Example 125

### 2′-[6-amino-5-(trifluoromethyl)pyridin-3-yl]-N-[(2-fluorophenyl)methyl]-5′,6′-dihydrospiro[azetidine-3,4′-pyrrolo[1,2-b]pyrazole]-1-carboxamide

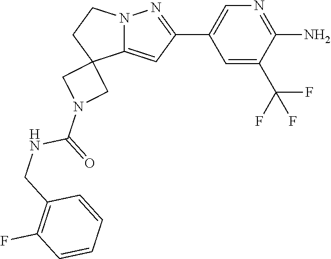 [SMILES: Nc1ncc(-c2cc3n(n2)CCC32CN(C(=O)NCc3ccccc3F)C2)cc1C(F)(F)F]

Example 125 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 1-(2-fluorophenyl)methanamine (36.4 mg, 291 μmol).

LC-MS (Method 1): Rt=1.03 min; MS (ESIpos): m/z=461 [M+H]⁺

¹H NMR (400 MHz, DMSO-d₆) δ ppm 2.86 (t, 2H) 4.00-4.09 (m, 4H) 4.21-4.31 (m, 4H) 6.55 (s, 2H) 6.74 (s, 1H) 7.07 (t, 1H) 7.24-7.33 (m, 3H) 7.37 (td, 1H) 8.04 (d, 1H) 8.62 (d, 1H)

### Example 126

### 2′-[6-amino-5-(trifluoromethyl)pyridin-3-yl]-N-[1-(pyridin-3-yl)cyclobutyl]-5′,6′-dihydrospiro[azetidine-3,4′-pyrrolo[1,2-b]pyrazole]-1-carboxamide

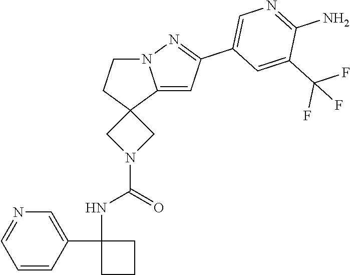 [SMILES: Nc1ncc(-c2cc3n(n2)CCC32CN(C(=O)NC3(c4cccnc4)CCC3)C2)cc1C(F)(F)F]

Example 126 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 1-(pyridin-3-yl)cyclobutan-1-amine-hydrochloride salt (64.3 mg, 291 μmol).

LC-MS (method 2): Rₜ=0.75 min; MS (ESIpos): m/z=484 [M+H]⁺

¹H NMR (400 MHz, DMSO-d₆) δ ppm 1.13 (d, 2H) 1.75-1.90 (m, 1H) 1.96-2.11 (m, 1H) 2.38-2.46 (m, 2H) 2.84 (t, 2H) 3.97-4.06 (m, 4H) 4.12 (t, 2H) 6.55 (s, 2H) 6.71 (s, 1H) 7.20 (s, 1H) 7.34 (ddd, 1H) 7.75-7.83 (m, 1H) 8.04 (d, 1H) 8.40 (dd, 1H) 8.61-8.67 (m, 2H)

### Example 127

### 2′-[6-amino-5-(trifluoromethyl)pyridin-3-yl]-N-[(4-fluorophenyl)methyl]-5′,6′-dihydrospiro[azetidine-3,4′-pyrrolo[1,2-b]pyrazole]-1-carboxamide

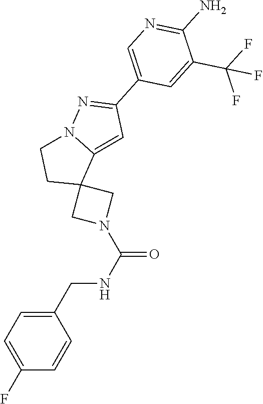 [SMILES: Nc1ncc(-c2cc3n(n2)CCC32CN(C(=O)NCc3ccc(F)cc3)C2)cc1C(F)(F)F]

Example 127 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 1-(4-fluorophenyl)methanamine (36.4 mg, 291 μmol).

LC-MS (Method 1): Rt=1.02 min; MS (ESIpos): m/z=461 [M+H]⁺

¹H NMR (400 MHz, DMSO-d₆) δ ppm 2.85 (t, 2H) 4.00-4.08 (m, 4H) 4.13 (t, 2H) 4.20 (d, 2H) 6.55 (s, 2H) 6.73 (s, 1H) 7.07-7.11 (m, 1H) 7.14 (t, 2H) 7.31 (dd, 2H) 8.04 (d, 1H) 8.62 (d, 1H)

### Example 128

### 2′-[6-amino-5-(trifluoromethyl)pyridin-3-yl]-N-[2-(pyridin-4-yl)propan-2-yl]-5′,6′-dihydrospiro[azetidine-3,4′-pyrrolo[1,2-b]pyrazole]-1-carboxamide

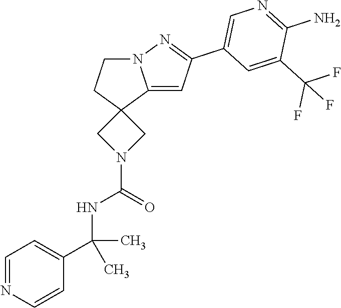 [SMILES: CC(C)(NC(=O)N1CC2(CCn3nc(-c4cnc(N)c(C(F)(F)F)c4)cc32)C1)c1ccncc1]

Example 128 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 2-(pyridin-4-yl)propan-2-amine (39.6 mg, 291 μmol).

LC-MS (Method 1): Rt=0.93 min; MS (ESIpos): m/z=472 [M+H]⁺

¹H NMR (400 MHz, DMSO-d₆) δ ppm 1.53 (s, 6H) 2.85 (t, 2H) 3.99-4.07 (m, 4H) 4.13 (t, 2H) 6.55 (s, 2H) 6.69-6.75 (m, 2H) 7.31-7.34 (m, 2H) 8.05 (d, 1H) 8.41-8.49 (m, 2H) 8.64 (d, 1H)

### Example 129

### 2′-[6-amino-5-(trifluoromethyl)pyridin-3-yl]-N-[2-(3-fluorophenyl)propan-2-yl]-5′,6′-dihydrospiro[azetidine-3,4′-pyrrolo[1,2-b]pyrazole]-1-carboxamide

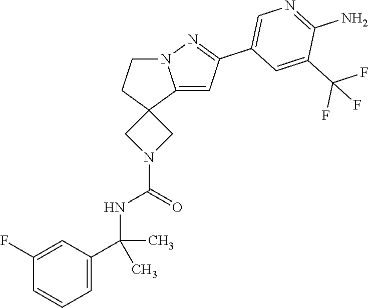 [SMILES: CC(C)(NC(=O)N1CC2(CCn3nc(-c4cnc(N)c(C(F)(F)F)c4)cc32)C1)c1cccc(F)c1]

Example 129 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 2-(3-fluorophenyl)propan-2-amine (44.6 mg, 291 μmol).

LC-MS (Method 1): Rt=1.15 min; MS (ESIpos): m/z=489 [M+H]⁺

¹H NMR (400 MHz, DMSO-d₆) δ ppm 1.55 (s, 6H) 2.85 (t, 2H) 3.99-4.08 (m, 4H) 4.13 (t, 2H) 6.55 (s, 2H) 6.63 (s, 1H) 6.71 (s, 1H) 6.99 (td, 1H) 7.13 (dt, 1H) 7.19 (d, 1H) 7.30-7.36 (m, 1H) 8.05 (d, 1H) 8.63 (d, 1H)

### Example 130

### 2′-[6-amino-5-(trifluoromethyl)pyridin-3-yl]-N-[(1S)-1-(3-chloropyridin-4-yl)ethyl]-5′,6′-dihydrospiro[azetidine-3,4′-pyrrolo[1,2-b]pyrazole]-1-carboxamide

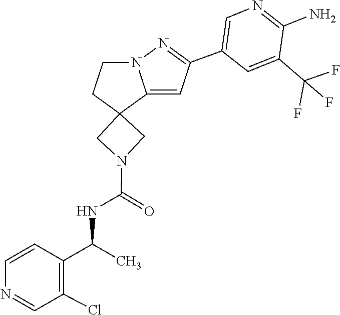 [SMILES: C[C@H](NC(=O)N1CC2(CCn3nc(-c4cnc(N)c(C(F)(F)F)c4)cc32)C1)c1ccncc1Cl]

Example 130 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and (1S)-1-(3-chloropyridin-4-yl)ethan-1-amine-hydrochloride salt (56.2 mg, 291 μmol).

¹H-NMR (400 MHz, DMSO-d₆): δ [ppm]=1.34 (d, 3H), 2.86 (t, 2H), 4.00-4.17 (m, 6H), 5.04 (quin, 1H), 6.55 (s, 2H), 6.73 (s, 1H), 7.19 (d, 1H), 7.49 (d, 1H), 8.04 (d, 1H), 8.52 (d, 1H), 8.55 (s, 1H), 8.62 (d, 1H).

### Example 131

### 2′-[6-amino-5-(trifluoromethyl)pyridin-3-yl]-N-[(1-methyl-2-oxabicyclo[2.1.1]hexan-4-yl)methyl]-5′,6′-dihydrospiro[azetidine-3,4′-pyrrolo[1,2-b]pyrazole]-1-carboxamide

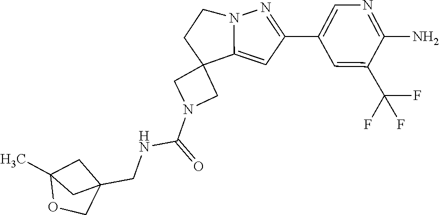 [SMILES: CC12CC(CNC(=O)N3CC4(CCn5nc(-c6cnc(N)c(C(F)(F)F)c6)cc54)C3)(CO1)C2]

Example 131 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 1-(1-methyl-2-oxabicyclo[2.1.1]hexan-4-yl)methanamine (37.0 mg, 291 μmol).

LC-MS (method 1): Rₜ=0.88 min; MS (ESIpos): m/z=463 [M+H]⁺

¹H-NMR (400 MHz, DMSO-d6) δ[ppm]: 1.139 (0.70), 1.155 (0.78), 1.288 (0.51), 1.312 (16.00), 1.327 (0.47), 1.350 (2.46), 1.354 (2.52), 1.361 (2.81), 1.365 (2.87), 1.489 (2.66), 1.493 (2.51), 1.500 (2.25), 1.504 (2.31), 2.518 (1.78), 2.523 (1.21), 2.828 (1.43), 2.846 (2.36), 2.863 (1.55), 3.310 (3.16), 3.325 (4.00), 3.469 (8.56), 3.970 (0.73), 3.990 (8.50), 4.013 (0.75), 4.108 (1.56), 4.125 (2.46), 4.143 (1.53), 6.546 (3.75), 6.594 (0.77), 6.609 (1.61), 6.624 (0.76), 6.718 (6.91), 8.037 (2.16), 8.042 (2.21), 8.618 (2.03), 8.622 (2.04).

### Example 132

### 2′-[6-amino-5-(trifluoromethyl)pyridin-3-yl]-N-{[3-(trifluoromethyl)bicyclo[1.1.1]pentan-1-yl]methyl}-5′,6′-dihydrospiro[azetidine-3,4′-pyrrolo[1,2-b]pyrazole]-1-carboxamide

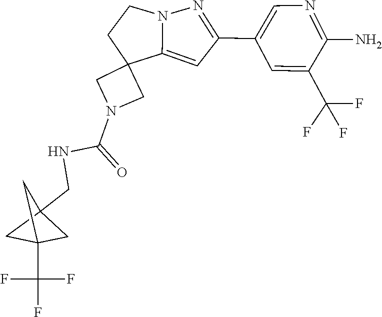 [SMILES: Nc1ncc(-c2cc3n(n2)CCC32CN(C(=O)NCC34CC(C(F)(F)F)(C3)C4)C2)cc1C(F)(F)F]

Example 132 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 1-[3-(trifluoromethyl)bicyclo[1.1.1]pentan-1-yl]methanamine-hydrochloride salt (58.7 mg, 291 μmol).

LC-MS (method 1): Rₜ=1.13 min; MS (ESIpos): m/z=501 [M+H]⁺

¹H-NMR (400 MHz, DMSO-d6) δ[ppm]: 1.833 (16.00), 2.074 (1.07), 2.518 (0.74), 2.523 (0.49), 2.831 (0.93), 2.848 (1.53), 2.865 (1.00), 3.163 (1.90), 3.178 (1.90), 3.976 (0.60), 3.996 (4.60), 4.000 (4.46), 4.021 (0.58), 4.109 (1.02), 4.127 (1.60), 4.143 (0.98), 6.546 (2.40), 6.596 (0.51), 6.610 (1.08), 6.625 (0.49), 6.713 (4.80), 8.034 (1.40), 8.040 (1.44), 8.616 (1.32), 8.620 (1.32).

### Example 133

### 2′-[6-amino-5-(trifluoromethyl)pyridin-3-yl]-N-[2-(2-chlorophenyl)propan-2-yl]-5′,6′-dihydrospiro[azetidine-3,4′-pyrrolo[1,2-b]pyrazole]-1-carboxamide

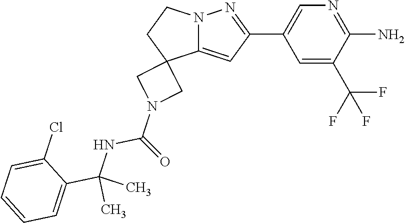 [SMILES: CC(C)(NC(=O)N1CC2(CCn3nc(-c4cnc(N)c(C(F)(F)F)c4)cc32)C1)c1ccccc1Cl]

Example 133 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 2-(2-chlorophenyl)propan-2-amine-hydrochloride salt (60.0 mg, 291 μmol).

¹H-NMR (400 MHz, DMSO-d₆): δ [ppm]=1.68 (s, 6H), 2.83 (t, 2H), 4.00 (s, 4H), 4.12 (t, 2H), 6.55 (s, 2H), 6.63-6.69 (m, 2H), 7.18-7.23 (m, 1H), 7.27 (td, 1H), 7.34 (dd, 1H), 7.47 (dd, 1H), 8.05 (d, 1H), 8.63 (d, 1H).

### Example 134

### 2′-[6-amino-5-(trifluoromethyl)pyridin-3-yl]-N-[(1R)-1-(3-fluorophenyl)ethyl]-5′,6′-dihydrospiro[azetidine-3,4′-pyrrolo[1,2-b]pyrazole]-1-carboxamide

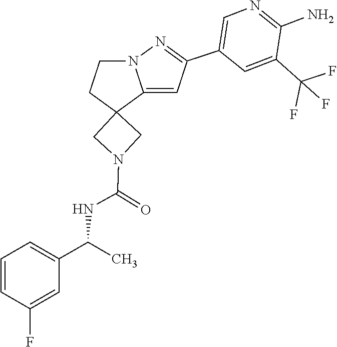 [SMILES: C[C@@H](NC(=O)N1CC2(CCn3nc(-c4cnc(N)c(C(F)(F)F)c4)cc32)C1)c1cccc(F)c1]

Example 134 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and (1R)-1-(3-fluorophenyl)ethan-1-amine (40.5 mg, 291 μmol).

¹H-NMR (400 MHz, DMSO-d₆): δ [ppm]=1.36 (d, 3H), 2.85 (t, 2H), 3.98-4.09 (m, 4H), 4.13 (t, 2H), 4.76-4.87 (m, 1H), 6.55 (s, 2H), 6.72 (s, 1H), 6.93 (d, 1H), 7.00-7.07 (m, 1H), 7.13-7.21 (m, 2H), 7.29-7.40 (m, 1H), 8.04 (d, 1H), 8.62 (d, 1H).

### Example 135

### 2′-[6-amino-5-(trifluoromethyl)pyridin-3-yl]-N-[2-(3-fluoropyridin-2-yl)propan-2-yl]-5′,6′-dihydrospiro[azetidine-3,4′-pyrrolo[1,2-b]pyrazole]-1-carboxamide

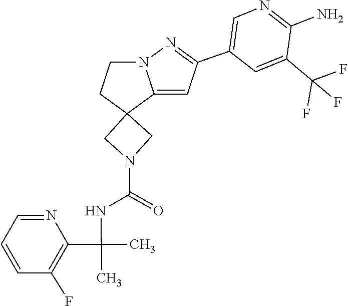 [SMILES: CC(C)(NC(=O)N1CC2(CCn3nc(-c4cnc(N)c(C(F)(F)F)c4)cc32)C1)c1ncccc1F]

Example 135 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 2-(3-fluoropyridin-2-yl)propan-2-amine (44.9 mg, 291 μmol).

LC-MS (method 1): Rₜ=1.05 min; MS (ESIneg): m/z=488 [M−H]⁻

¹H-NMR (400 MHz, DMSO-d6) δ[ppm]: 1.612 (16.00), 2.518 (0.79), 2.523 (0.49), 2.823 (1.40), 2.840 (2.28), 2.858 (1.49), 3.970 (0.61), 3.992 (8.57), 4.014 (0.61), 4.111 (1.54), 4.128 (2.41), 4.145 (1.46), 5.758 (11.42), 6.545 (3.70), 6.701 (7.18), 6.802 (3.32), 7.316 (0.74), 7.325 (0.87), 7.328 (0.84), 7.336 (1.58), 7.346 (0.98), 7.348 (1.08), 7.357 (0.86), 7.561 (0.91), 7.564 (0.92), 7.582 (0.81), 7.585 (0.81), 7.592 (0.90), 7.595 (0.91), 7.613 (0.78), 7.616 (0.77), 8.048 (2.15), 8.053 (2.17), 8.307 (0.92), 8.311 (1.49), 8.315 (1.07), 8.318 (1.01), 8.322 (1.44), 8.326 (0.90), 8.628 (2.00), 8.633 (1.98).

### Example 136

### 2′-[6-amino-5-(trifluoromethyl)pyridin-3-yl]-N-[1-(2-chlorophenyl)cyclobutyl]-5′,6′-dihydrospiro[azetidine-3,4′-pyrrolo[1,2-b]pyrazole]-1-carboxamide

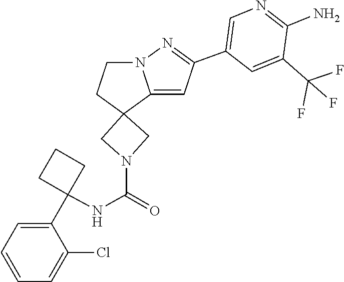 [SMILES: Nc1ncc(-c2cc3n(n2)CCC32CN(C(=O)NC3(c4ccccc4Cl)CCC3)C2)cc1C(F)(F)F]

Example 136 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 1-(2-chlorophenyl)cyclobutan-1-amine-hydrochloride salt (63.5 mg, 291 μmol).

¹H-NMR (400 MHz, DMSO-d₆): δ [ppm]=1.63-1.76 (m, 1H), 1.99-2.14 (m, 1H), 2.57-2.69 (m, 2H), 2.80 (t, 2H), 3.91-3.99 (m, 4H), 4.10 (t, 2H), 6.54 (s, 2H), 6.64 (s, 1H), 7.02 (s, 1H), 7.17-7.29 (m, 2H), 7.33 (dd, 1H), 7.54 (dd, 1H), 8.03 (d, 1H), 8.60 (d, 1H).

### Example 137

### 2′-[6-amino-5-(trifluoromethyl)pyridin-3-yl]-N-[1-(3-fluorophenyl)cyclobutyl]-5′,6′-dihydrospiro[azetidine-3,4′-pyrrolo[1,2-b]pyrazole]-1-carboxamide

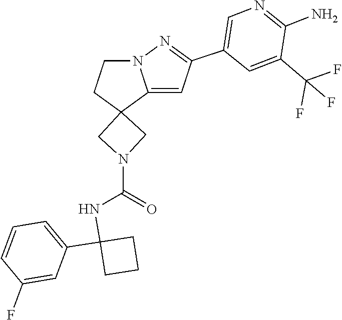 [SMILES: Nc1ncc(-c2cc3n(n2)CCC32CN(C(=O)NC3(c4cccc(F)c4)CCC3)C2)cc1C(F)(F)F]

Example 137 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 1-(3-fluorophenyl)cyclobutan-1-amine-hydrochloride salt (58.7 mg, 291 μmol).

¹H-NMR (400 MHz, DMSO-d₆): δ [ppm]=1.75 (dddd, 1H), 1.98-2.12 (m, 3H), 2.81 (t, 2H), 3.90-4.02 (m, 4H), 4.11 (t, 2H), 6.54 (s, 2H), 6.67 (s, 1H), 7.04-7.15 (m, 3H), 7.24 (tdd, 1H), 7.43 (td, 1H), 8.03 (d, 1H), 8.61 (d, 1H).

### Example 138

### 2′-[6-amino-5-(trifluoromethyl)pyridin-3-yl]-N-[(1R)-1-phenylpropyl]-5′,6′-dihydrospiro[azetidine-3,4′-pyrrolo[1,2-b]pyrazole]-1-carboxamide

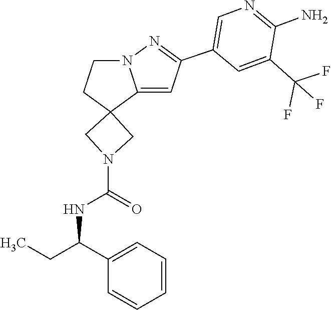 [SMILES: CC[C@@H](NC(=O)N1CC2(CCn3nc(-c4cnc(N)c(C(F)(F)F)c4)cc32)C1)c1ccccc1]

Example 138 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and (1R)-1-phenylpropan-1-amine (39.3 mg, 291 μmol).

¹H-NMR (400 MHz, DMSO-d₆): δ [ppm]=0.86 (t, 3H), 1.61-1.75 (m, 2H), 2.84 (t, 2H), 3.97-4.06 (m, 4H), 4.12 (t, 2H), 4.49-4.59 (m, 1H), 6.54 (s, 2H), 6.71 (s, 1H), 6.83 (d, 1H), 7.16-7.25 (m, 1H), 7.27-7.36 (m, 4H), 8.04 (d, 1H), 8.61 (d, 1H).

### Example 139

### 2′-[6-amino-5-(trifluoromethyl)pyridin-3-yl]-N-[(pyrimidin-4-yl)methyl]-5′,6′-dihydrospiro[azetidine-3,4′-pyrrolo[1,2-b]pyrazole]-1-carboxamide

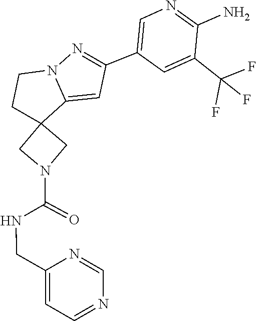 [SMILES: Nc1ncc(-c2cc3n(n2)CCC32CN(C(=O)NCc3ccncn3)C2)cc1C(F)(F)F]

Example 139 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 1-(pyrimidin-4-yl)methanamine (31.8 mg, 291 μmol).

¹H-NMR (400 MHz, DMSO-d₆): δ [ppm]=2.88 (t, 2H), 4.05-4.20 (m, 6H), 4.30 (d, 2H), 6.56 (s, 2H), 6.76 (s, 1H), 7.26 (t, 1H), 7.44 (dd, 1H), 8.05 (d, 1H), 8.63 (d, 1H), 8.76 (d, 1H), 9.09 (d, 1H).

### Example 140

### 2′-[6-amino-5-(trifluoromethyl)pyridin-3-yl]-N-[(3-fluoropyridin-2-yl)methyl]-5′,6′-dihydrospiro[azetidine-3,4′-pyrrolo[1,2-b]pyrazole]-1-carboxamide

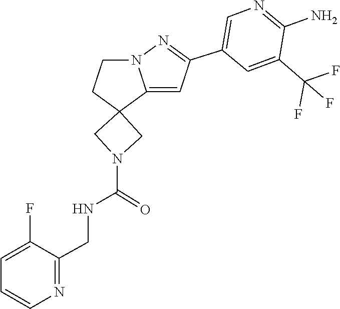 [SMILES: Nc1ncc(-c2cc3n(n2)CCC32CN(C(=O)NCc3ncccc3F)C2)cc1C(F)(F)F]

Example 140 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 1-(3-fluoropyridin-2-yl)methanamine (36.7 mg, 291 μmol).

¹H-NMR (400 MHz, DMSO-d₆): δ [ppm]=4.04 (s, 4H), 4.13 (t, 2H), 4.40 (dd, 2H), 6.55 (s, 2H), 6.72 (s, 1H), 7.00 (t, 1H), 7.39 (dt, 1H), 7.67 (ddd, 1H), 8.04 (d, 1H), 8.39 (dt, 1H), 8.62 (d, 1H).

### Example 141

### 2′-[6-amino-5-(trifluoromethyl)pyridin-3-yl]-N-[(pyrimidin-5-yl)methyl]-5′,6′-dihydrospiro[azetidine-3,4′-pyrrolo[1,2-b]pyrazole]-1-carboxamide

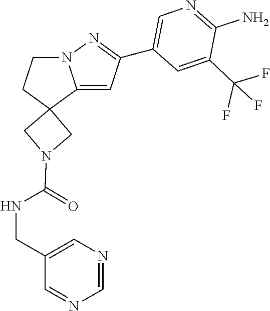 [SMILES: Nc1ncc(-c2cc3n(n2)CCC32CN(C(=O)NCc3cncnc3)C2)cc1C(F)(F)F]

Example 141 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 1-(pyrimidin-5-yl)methanamine (31.8 mg, 291 μmol).

¹H-NMR (400 MHz, DMSO-d₆): δ [ppm]=2.86 (t, 2H), 3.99-4.08 (m, 4H), 4.13 (t, 2H), 4.25 (d, 2H), 6.55 (s, 2H), 6.73 (s, 1H), 7.17 (t, 1H), 8.04 (d, 1H), 8.61 (d, 1H), 8.73 (s, 2H), 9.08 (s, 1H).

### Example 142

### 2′-[6-amino-5-(trifluoromethyl)pyridin-3-yl]-N-[(3-chloropyridin-4-yl)methyl]-5′,6′-dihydrospiro[azetidine-3,4′-pyrrolo[1,2-b]pyrazole]-1-carboxamide

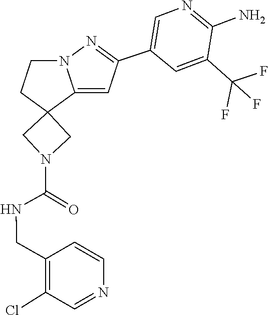 [SMILES: Nc1ncc(-c2cc3n(n2)CCC32CN(C(=O)NCc3ccncc3Cl)C2)cc1C(F)(F)F]

Example 142 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 1-(3-chloropyridin-4-yl)methanamine-hydrochloride salt (62.7 mg, 291 μmol).

¹H-NMR (400 MHz, DMSO-d₆): δ [ppm]=2.89 (t, 2H), 4.04-4.18 (m, 6H), 4.31 (d, 2H), 6.56 (s, 2H), 6.76 (s, 1H), 7.22 (t, 1H), 7.38 (d, 1H), 8.05 (d, 1H), 8.52 (d, 1H), 8.57 (s, 1H), 8.63 (d, 1H).

### Example 143

### 2′-[6-amino-5-(trifluoromethyl)pyridin-3-yl]-N-[(3-fluorophenyl)methyl]-5′,6′-dihydrospiro[azetidine-3,4′-pyrrolo[1,2-b]pyrazole]-1-carboxamide

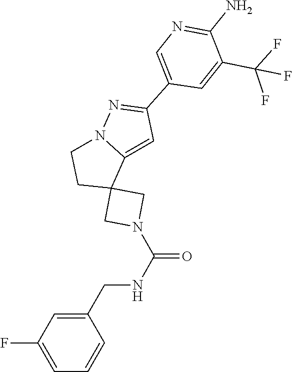 [SMILES: Nc1ncc(-c2cc3n(n2)CCC32CN(C(=O)NCc3cccc(F)c3)C2)cc1C(F)(F)F]

Example 143 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 1-(3-fluorophenyl)methanamine (36.4 mg, 291 μmol).

¹H-NMR (400 MHz, DMSO-d₆): δ [ppm]=2.86 (t, 2H), 4.01-4.08 (m, 4H), 4.13 (t, 2H), 4.24 (d, 2H), 6.55 (s, 2H), 6.73 (s, 1H), 6.99-7.17 (m, 4H), 7.33-7.41 (m, 1H), 8.04 (d, 1H), 8.62 (d, 1H).

### Example 144

### 2′-[6-amino-5-(trifluoromethyl)pyridin-3-yl]-N-[(pyridin-3-yl)methyl]-5′,6′-dihydrospiro[azetidine-3,4′-pyrrolo[1,2-b]pyrazole]-1-carboxamide

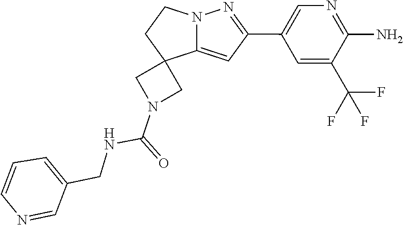 [SMILES: Nc1ncc(-c2cc3n(n2)CCC32CN(C(=O)NCc3cccnc3)C2)cc1C(F)(F)F]

Example 144 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 1-(pyridin-3-yl)methanamine (31.5 mg, 291 μmol).

¹H-NMR (400 MHz, DMSO-d₆): δ [ppm]=2.86 (t, 2H), 3.99-4.08 (m, 4H), 4.13 (t, 2H), 4.25 (d, 2H), 6.55 (s, 2H), 6.73 (s, 1H), 7.14 (t, 1H), 7.34-7.39 (m, 1H), 7.69 (dt, 1H), 8.04 (d, 1H), 8.45 (dd, 1H), 8.50 (d, 1H), 8.62 (d, 1H).

### Example 145

### (rac)-2-[6-amino-5-(trifluoromethyl)pyridin-3-yl]-N-benzyl-6,7-dihydrospiro[pyrazolo[5,1-c][1,4]oxazine-4,3′-pyrrolidine]-1′-carboxamide

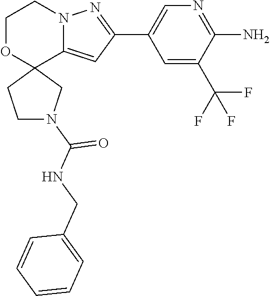 [SMILES: Nc1ncc(-c2cc3n(n2)CCOC32CCN(C(=O)NCc3ccccc3)C2)cc1C(F)(F)F]

Example 145 was prepared in analogy to Example 31 using (rac)-5-[6,7-dihydrospiro[pyrazolo[5,1-c][1,4]oxazine-4,3′-pyrrolidin]-2-yl]-3-(trifluoromethyl)pyridin-2-amine (75.0 mg, 221 μmol) and 1-phenylmethanamine (29 μL, 270 μmol).

1H-NMR (400 MHz, DMSO-d6) δ[ppm]: 1.066 (0.44), 2.261 (0.54), 2.336 (0.55), 2.358 (0.52), 2.518 (3.87), 2.522 (2.44), 3.394 (0.52), 3.403 (0.75), 3.419 (0.78), 3.429 (0.60), 3.445 (0.50), 3.468 (1.49), 3.497 (1.90), 3.578 (2.70), 3.601 (2.27), 3.625 (1.19), 3.807 (0.99), 3.835 (0.86), 4.068 (0.58), 4.084 (0.68), 4.102 (0.78), 4.118 (0.97), 4.125 (1.61), 4.138 (2.83), 4.221 (9.96), 4.235 (10.31), 4.258 (2.32), 6.433 (2.58), 6.620 (0.70), 6.758 (5.12), 6.807 (0.52), 6.821 (1.01), 6.836 (0.50), 7.200 (2.37), 7.204 (1.71), 7.212 (2.06), 7.218 (6.45), 7.223 (2.75), 7.236 (10.46), 7.238 (11.01), 7.255 (14.73), 7.259 (9.99), 7.283 (4.86), 7.291 (16.00), 7.297 (4.71), 7.308 (11.40), 7.310 (13.14), 7.324 (2.34), 7.328 (5.33), 8.019 (1.97), 8.024 (1.98), 8.581 (1.79), 8.585 (1.79).

### Example 146

### (rac)-2-[6-amino-5-(trifluoromethyl)pyridin-3-yl]-N-[(1R)-1-(2-methylpyrimidin-5-yl)ethyl]-6,7-dihydrospiro[pyrazolo[5,1-c][1,4]oxazine-4,3′-pyrrolidine]-1′-carboxamide (Mixture of Stereoisomers)

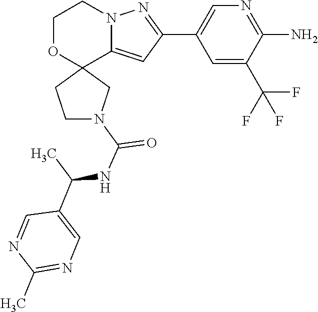 [SMILES: Cc1ncc([C@@H](C)NC(=O)N2CCC3(C2)OCCn2nc(-c4cnc(N)c(C(F)(F)F)c4)cc23)cn1]

Example 146 was prepared in analogy to Example 31 using (rac)-5-[6,7-dihydrospiro[pyrazolo[5,1-c][1,4]oxazine-4,3′-pyrrolidin]-2-yl]-3-(trifluoromethyl)pyridin-2-amine-hydrochloride salt (75.0 mg, 200 μmol) and (1R)-1-(2-methylpyrimidin-5-yl)ethan-1-amine (30.1 mg, 220 μmol).

¹H-NMR (400 MHz, DMSO-d6) δ[ppm]: 1.232 (0.66), 1.407 (7.43), 1.425 (7.45), 2.236 (0.61), 2.323 (1.38), 2.327 (1.97), 2.331 (1.60), 2.518 (5.63), 2.523 (3.77), 2.540 (4.58), 2.579 (16.00), 2.665 (0.96), 2.669 (1.32), 2.674 (0.90), 3.386 (1.53), 3.405 (0.94), 3.443 (0.92), 3.471 (0.99), 3.487 (0.88), 3.516 (0.94), 3.551 (0.50), 3.573 (0.83), 3.595 (0.79), 3.617 (0.83), 3.754 (0.94), 3.785 (0.77), 3.810 (0.90), 3.837 (0.79), 4.077 (0.99), 4.083 (1.01), 4.106 (1.62), 4.118 (1.93), 4.132 (4.30), 4.804 (0.64), 4.813 (0.75), 4.822 (1.03), 4.831 (1.03), 4.840 (0.75), 4.849 (0.66), 6.600 (6.31), 6.617 (2.26), 6.637 (1.05), 6.747 (3.59), 6.754 (4.56), 8.007 (3.24), 8.171 (2.41), 8.575 (3.20), 8.640 (9.38), 8.646 (9.14).

### Example 147

### (rac)-2-[6-amino-5-(trifluoromethyl)pyridin-3-yl]-N-[(1R)-1-(pyridin-3-yl)ethyl]-6,7-dihydrospiro[pyrazolo[5,1-c][1,4]oxazine-4,3′-pyrrolidine]-1′-carboxamide (Mixture of Stereoisomers)

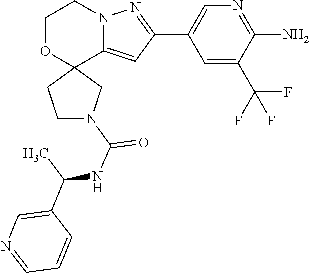 [SMILES: C[C@@H](NC(=O)N1CCC2(C1)OCCn1nc(-c3cnc(N)c(C(F)(F)F)c3)cc12)c1cccnc1]

Example 147 was prepared in analogy to Example 31 using (rac)-5-[6,7-dihydrospiro[pyrazolo[5,1-c][1,4]oxazine-4,3′-pyrrolidin]-2-yl]-3-(trifluoromethyl)pyridin-2-amine-hydrochloride salt (75.0 mg, 200 μmol) and (1R)-1-(pyridin-3-yl)ethan-1-amine-hydrochloride salt (42.8 mg, 220 μmol).

¹H-NMR (400 MHz, DMSO-d₆): δ [ppm]=1.40 (d, 3H), 2.16-2.29 (m, 1H), 2.30-2.40 (m, 1H), 3.49 (br t, 2H), 3.54-3.67 (m, 1H), 3.81 (dd, 1H), 4.01-4.19 (m, 4H), 4.80-4.93 (m, 1H), 6.55-6.64 (m, 3H), 6.75 (d, 1H), 7.29-7.37 (m, 1H), 7.74 (dd, 1H), 8.01 (s, 1H), 8.41 (d, 1H), 8.52-8.60 (m, 2H).

### Example 148

### 2′-[6-amino-5-(trifluoromethyl)pyridin-3-yl]-N-benzyl-5′,6′-dihydrospiro[azetidine-3,4′-pyrrolo[1,2-b]pyrazole]-1-carboxamide

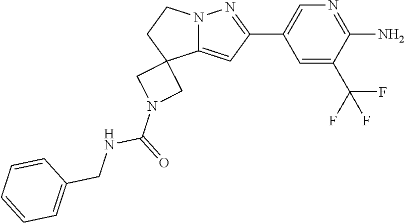 [SMILES: Nc1ncc(-c2cc3n(n2)CCC32CN(C(=O)NCc3ccccc3)C2)cc1C(F)(F)F]

Example 148 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 1-phenylmethanamine (32 μL, 290 μmol).

1H-NMR (400 MHz, DMSO-d6) δ[ppm]: 2.518 (4.48), 2.522 (2.90), 2.840 (2.15), 2.857 (3.37), 2.874 (2.25), 4.014 (1.40), 4.034 (9.82), 4.039 (9.51), 4.060 (1.40), 4.112 (2.31), 4.130 (3.59), 4.147 (2.19), 4.221 (15.88), 4.236 (16.00), 6.419 (1.85), 6.434 (3.43), 6.449 (1.78), 6.546 (5.54), 6.735 (10.71), 7.063 (1.14), 7.078 (2.45), 7.093 (1.09), 7.200 (1.89), 7.204 (1.26), 7.211 (1.58), 7.218 (5.37), 7.223 (2.51), 7.227 (2.94), 7.236 (8.98), 7.238 (9.57), 7.249 (3.73), 7.254 (12.47), 7.259 (8.50), 7.271 (2.60), 7.288 (8.44), 7.291 (14.52), 7.297 (4.14), 7.307 (12.53), 7.310 (11.42), 7.312 (9.04), 7.324 (6.06), 7.328 (6.10), 7.340 (0.77), 7.343 (1.62), 8.039 (3.24), 8.044 (3.27), 8.618 (2.98), 8.622 (2.94).

### Example 149

2-[6-amino-5-(trifluoromethyl)pyridin-3-yl]-N-[2-(2-fluorophenyl)propan-2-yl]-6,7-dihydrospiro[pyrazolo[5,1-c][1,4]oxazine-4,3′-pyrrolidine]-1′-carboxamide (Enantiomer 1)

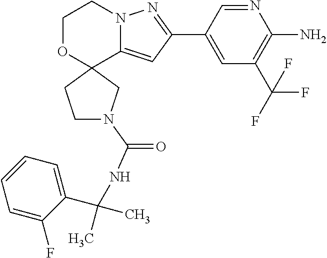 [SMILES: CC(C)(NC(=O)N1CCC2(C1)OCCn1nc(-c3cnc(N)c(C(F)(F)F)c3)cc12)c1ccccc1F]

Example 149 was prepared in analogy to Example 31 using 5-[6,7-dihydrospiro[pyrazolo[5,1-c][1,4]oxazine-4,3′-pyrrolidin]-2-yl]-3-(trifluoromethyl)pyridin-2-amine-hydrochloride salt (86.0 mg, 229 μmol) and 2-(2-fluorophenyl)propan-2-amine (42.1 mg, 275 μmol).

[α]²⁰_{D}: −53.3° (c=1.00, DMSO)

¹H-NMR (400 MHz, DMSO-d6) δ[ppm]: 1.137 (0.43), 1.232 (1.06), 1.629 (16.00), 1.635 (15.74), 2.085 (11.75), 2.221 (0.85), 2.247 (1.20), 2.269 (0.64), 2.318 (1.38), 2.323 (1.87), 2.327 (2.70), 2.332 (2.27), 2.346 (0.67), 2.362 (0.56), 2.518 (5.33), 2.523 (3.85), 2.660 (0.54), 2.665 (1.21), 2.669 (1.73), 2.673 (1.20), 2.679 (0.53), 3.363 (0.56), 3.388 (1.18), 3.404 (1.28), 3.430 (0.55), 3.471 (1.87), 3.500 (2.20), 3.570 (0.92), 3.591 (1.64), 3.613 (0.73), 3.778 (1.95), 3.807 (1.66), 4.069 (0.62), 4.084 (1.26), 4.101 (1.81), 4.119 (2.91), 4.130 (5.03), 4.142 (5.18), 4.155 (1.63), 5.759 (11.02), 6.114 (5.82), 6.600 (7.28), 6.760 (12.67), 7.031 (1.40), 7.034 (1.56), 7.051 (1.76), 7.054 (1.95), 7.062 (1.34), 7.065 (1.66), 7.075 (1.89), 7.078 (1.85), 7.083 (1.67), 7.086 (2.25), 7.093 (3.68), 7.097 (2.89), 7.112 (2.51), 7.116 (2.14), 7.191 (0.94), 7.195 (1.08), 7.203 (1.07), 7.207 (1.44), 7.215 (1.38), 7.222 (1.33), 7.227 (1.35), 7.233 (0.72), 7.242 (0.63), 7.246 (0.60), 7.299 (1.48), 7.303 (1.45), 7.321 (2.26), 7.341 (1.28), 7.345 (1.11), 8.013 (4.42), 8.018 (4.44), 8.582 (4.14), 8.586 (4.04).

### Example 150

### 2-[6-amino-5-(trifluoromethyl)pyridin-3-yl]-N-[2-(3-chloropyridin-4-yl)propan-2-yl]-6,7-dihydrospiro[pyrazolo[5,1-c][1,4]oxazine-4,3′-pyrrolidine]-1′-carboxamide (Enantiomer 1)

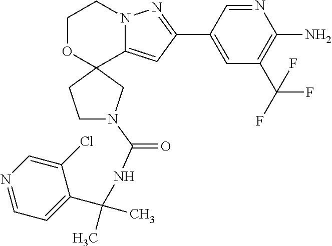 [SMILES: CC(C)(NC(=O)N1CCC2(C1)OCCn1nc(-c3cnc(N)c(C(F)(F)F)c3)cc12)c1ccncc1Cl]

Example 150 was prepared in analogy to Example 31 using 5-[6,7-dihydrospiro[pyrazolo[5,1-c][1,4]oxazine-4,3′-pyrrolidin]-2-yl]-3-(trifluoromethyl)pyridin-2-amine-hydrochloride salt (86.0 mg, 229 μmol) and 2-(3-chloropyridin-4-yl)propan-2-amine (46.9 mg, 275 μmol).

[α]²⁰_{D}: −59.8° (c=1.00, DMSO)

LC-MS (method 1): Rₜ=1.01 min; MS (ESIpos): m/z=536 [M+H]⁺

¹H-NMR (400 MHz, DMSO-d6) δ[ppm]: 1.232 (0.48), 1.627 (0.50), 1.649 (7.71), 1.664 (7.17), 2.085 (2.63), 2.228 (0.59), 2.306 (0.47), 2.323 (1.34), 2.327 (1.53), 2.332 (1.07), 2.337 (0.65), 2.518 (3.65), 2.523 (2.59), 2.665 (0.86), 2.669 (1.20), 2.673 (0.83), 3.375 (0.52), 3.392 (0.52), 3.444 (0.83), 3.472 (0.97), 3.562 (0.68), 3.746 (0.82), 3.776 (0.68), 4.071 (0.60), 4.085 (0.71), 4.102 (0.94), 4.108 (1.05), 4.121 (2.05), 4.133 (2.98), 5.759 (16.00), 6.354 (2.84), 6.599 (3.52), 6.748 (6.20), 7.423 (2.45), 7.436 (2.48), 8.012 (2.13), 8.017 (2.13), 8.397 (3.95), 8.410 (3.78), 8.428 (7.25), 8.582 (1.98), 8.586 (1.95).

### Example 151

### 2-[6-amino-5-(trifluoromethyl)pyridin-3-yl]-N-[2-(2-fluorophenyl)propan-2-yl]-6,7-dihydrospiro[pyrazolo[5,1-c][1,4]oxazine-4,3′-pyrrolidine]-1′-carboxamide (Enantiomer 2)

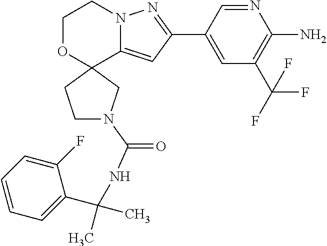 [SMILES: CC(C)(NC(=O)N1CCC2(C1)OCCn1nc(-c3cnc(N)c(C(F)(F)F)c3)cc12)c1ccccc1F]

Example 151 was prepared in analogy to Example 31 using 5-[6,7-dihydrospiro[pyrazolo[5,1-c][1,4]oxazine-4,3′-pyrrolidin]-2-yl]-3-(trifluoromethyl)pyridin-2-amine-hydrochloride salt (48.0 mg, 128 μmol) and 2-(2-fluorophenyl)propan-2-amine (23.5 mg, 153 μmol).

[α]²⁰_{D}: +46.8° (c=1.00, DMSO)

LC-MS (method 1): Rₜ=1.15 min; MS (ESIpos): m/z=519 [M+H]⁺

¹H-NMR (400 MHz, DMSO-d6) δ[ppm]: 1.232 (1.41), 1.629 (16.00), 1.635 (15.62), 2.220 (0.84), 2.246 (1.20), 2.269 (0.62), 2.318 (1.80), 2.323 (2.86), 2.327 (4.01), 2.332 (3.17), 2.363 (0.55), 2.518 (9.47), 2.523 (6.66), 2.660 (0.99), 2.665 (2.17), 2.669 (2.99), 2.673 (2.08), 2.678 (0.92), 3.363 (0.61), 3.387 (1.18), 3.405 (1.19), 3.430 (0.55), 3.471 (1.86), 3.499 (2.15), 3.569 (0.94), 3.591 (1.64), 3.614 (0.72), 3.778 (1.99), 3.807 (1.65), 4.069 (0.63), 4.084 (1.30), 4.101 (1.82), 4.119 (3.00), 4.130 (5.10), 4.142 (5.22), 5.759 (9.17), 6.114 (5.71), 6.600 (7.40), 6.760 (11.84), 7.031 (1.36), 7.034 (1.48), 7.051 (1.75), 7.054 (1.89), 7.065 (1.61), 7.075 (1.82), 7.078 (1.72), 7.086 (2.15), 7.094 (3.55), 7.097 (2.80), 7.113 (2.42), 7.116 (2.02), 7.191 (0.92), 7.195 (1.06), 7.203 (1.08), 7.208 (1.46), 7.215 (1.33), 7.222 (1.32), 7.227 (1.30), 7.233 (0.67), 7.242 (0.60), 7.246 (0.57), 7.299 (1.46), 7.303 (1.39), 7.321 (2.22), 7.341 (1.23), 7.345 (1.04), 8.013 (4.44), 8.018 (4.40), 8.581 (4.17), 8.586 (4.04).

### Example 152

### 2-[6-amino-5-(trifluoromethyl)pyridin-3-yl]-N-[2-(3-chloropyridin-4-yl)propan-2-yl]-6,7-dihydrospiro[pyrazolo[5,1-c][1,4]oxazine-4,3′-pyrrolidine]-1′-carboxamide (Enantiomer 2)

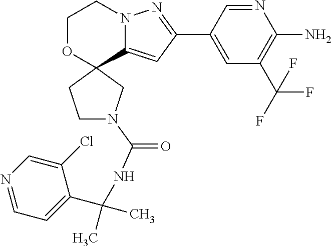 [SMILES: CC(C)(NC(=O)N1CC[C@@]2(C1)OCCn1nc(-c3cnc(N)c(C(F)(F)F)c3)cc12)c1ccncc1Cl]

Example 152 was prepared in analogy to Example 31 using 5-[6,7-dihydrospiro[pyrazolo[5,1-c][1,4]oxazine-4,3′-pyrrolidin]-2-yl]-3-(trifluoromethyl)pyridin-2-amine-hydrochloride salt (48.0 mg, 128 μmol) and 2-(3-chloropyridin-4-yl)propan-2-amine (26.2 mg, 153 μmol).

[α]²⁰_{D}: +59.6° (c=1.00, DMSO)

LC-MS (method 1): Rₜ=1.01 min; MS (ESIpos): m/z=536 [M+H]⁺

¹H-NMR (400 MHz, DMSO-d6) δ[ppm]: 0.852 (0.41), 1.232 (1.54), 1.649 (16.00), 1.664 (14.91), 2.195 (0.81), 2.202 (0.84), 2.228 (1.21), 2.252 (0.62), 2.306 (0.95), 2.323 (3.25), 2.327 (3.82), 2.332 (2.72), 2.336 (1.59), 2.518 (10.89), 2.523 (7.64), 2.660 (1.00), 2.665 (2.23), 2.669 (3.12), 2.673 (2.20), 2.678 (0.95), 3.374 (1.22), 3.392 (1.15), 3.418 (0.59), 3.444 (1.75), 3.472 (2.03), 3.541 (0.85), 3.563 (1.45), 3.585 (0.69), 3.746 (1.72), 3.776 (1.43), 4.055 (0.67), 4.069 (1.22), 4.086 (1.51), 4.108 (2.19), 4.121 (4.28), 4.133 (6.24), 5.759 (12.16), 6.354 (5.81), 6.599 (7.30), 6.748 (12.34), 7.423 (4.91), 7.436 (5.06), 8.012 (4.36), 8.017 (4.45), 8.397 (7.68), 8.410 (7.57), 8.428 (14.72), 8.581 (4.06), 8.586 (4.09).

### Example 153

### 2-[6-amino-5-(trifluoromethyl)pyridin-3-yl]-N-[(1R)-1-(4-cyanopyridin-2-yl)ethyl]-6,7-dihydrospiro[pyrazolo[5,1-c][1,4]oxazine-4,3′-pyrrolidine]-1′-carboxamide (Mixture of Stereoisomers)

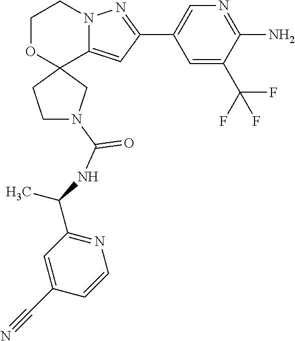 [SMILES: C[C@@H](NC(=O)N1CCC2(C1)OCCn1nc(-c3cnc(N)c(C(F)(F)F)c3)cc12)c1cc(C#N)ccn1]

Example 153 was prepared in analogy to Example 31 using (rac)-5-[6,7-dihydrospiro[pyrazolo[5,1-c][1,4]oxazine-4,3′-pyrrolidin]-2-yl]-3-(trifluoromethyl)pyridin-2-amine-hydrochloride salt (75 mg, 200 μmol) and 2-[(1R)-1-aminoethyl]pyridine-4-carbonitrile-hydrochloride salt (37.6 mg, 205 μmol).

¹H-NMR (400 MHz, DMSO-d₆): δ [ppm]=1.42 (d, 3H), 2.17-2.29 (m, 1H), 2.31-2.41 (m, 1H), 3.36-3.69 (m, 3H), 3.74-3.88 (m, 1H), 4.03-4.19 (m, 4H), 4.85-4.97 (m, 1H), 6.60 (s, 2H), 6.65 (dd, 1H), 6.76 (d, 1H), 8.01 (s, 1H), 8.26 (q, 1H), 8.58 (s, 1H), 8.87 (dt, 2H).

### Example 154

### (rac)-2-[6-amino-5-(trifluoromethyl)pyridin-3-yl]-N-[2-(1-methyl-1H-1,2,3-triazol-5-yl)propan-2-yl]-6,7-dihydrospiro[pyrazolo[5,1-c][1,4]oxazine-4,3′-pyrrolidine]-1′-carboxamide

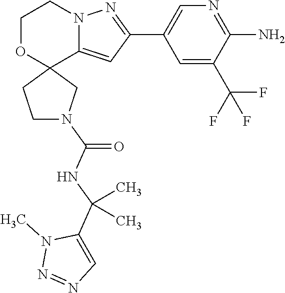 [SMILES: Cn1nncc1C(C)(C)NC(=O)N1CCC2(C1)OCCn1nc(-c3cnc(N)c(C(F)(F)F)c3)cc12]

Example 154 was prepared in analogy to Example 31 using (rac)-5-[6,7-dihydrospiro[pyrazolo[5,1-c][1,4]oxazine-4,3′-pyrrolidin]-2-yl]-3-(trifluoromethyl)pyridin-2-amine-hydrochloride salt (80.0 mg, 213 μmol) and 2-(1-methyl-1H-1,2,3-triazol-4-yl)propan-2-amine-hydrochloride salt (49.9 mg, 234 μmol).

LC-MS (method 2): Rₜ=0.82 min; MS (ESIpos): m/z=506 [M+H]⁺

¹H-NMR (400 MHz, DMSO-d6) δ[ppm]: 1.595 (9.55), 1.600 (9.66), 2.226 (0.52), 2.301 (0.45), 2.318 (0.71), 2.323 (0.76), 2.327 (0.88), 2.332 (0.76), 2.518 (2.20), 2.523 (1.48), 2.665 (0.47), 2.669 (0.65), 2.673 (0.45), 3.351 (0.56), 3.359 (0.64), 3.377 (0.60), 3.385 (0.42), 3.444 (1.00), 3.473 (1.14), 3.558 (0.47), 3.580 (0.86), 3.764 (0.99), 3.791 (0.84), 3.981 (16.00), 4.072 (0.54), 4.090 (0.76), 4.108 (1.33), 4.119 (1.83), 4.132 (2.89), 5.951 (2.63), 6.597 (3.32), 6.746 (5.53), 7.791 (6.40), 8.011 (2.02), 8.016 (2.05), 8.580 (1.89), 8.585 (1.87).

### Example 155

### (rac)-2-[6-amino-5-(trifluoromethyl)pyridin-3-yl]-N-[2-(pyridin-3-yl)propan-2-yl]-6,7-dihydrospiro[pyrazolo[5,1-c][1,4]oxazine-4,3′-pyrrolidine]-1′-carboxamide

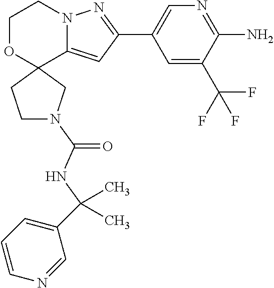 [SMILES: CC(C)(NC(=O)N1CCC2(C1)OCCn1nc(-c3cnc(N)c(C(F)(F)F)c3)cc12)c1cccnc1]

Example 155 was prepared in analogy to Example 31 using (rac)-5-[6,7-dihydrospiro[pyrazolo[5,1-c][1,4]oxazine-4,3′-pyrrolidin]-2-yl]-3-(trifluoromethyl)pyridin-2-amine-hydrochloride salt (80.0 mg, 213 μmol) and 2-(pyridin-3-yl)propan-2-amine (31.9 mg, 234 μmol).

¹H-NMR (400 MHz, DMSO-d6) δ[ppm]: 1.583 (16.00), 2.075 (0.43), 2.221 (0.55), 2.245 (0.77), 2.269 (0.41), 2.318 (1.11), 2.323 (1.59), 2.327 (1.97), 2.332 (1.75), 2.349 (0.46), 2.518 (4.88), 2.523 (3.29), 2.660 (0.43), 2.665 (1.06), 2.669 (1.49), 2.673 (1.01), 2.679 (0.46), 3.391 (0.79), 3.410 (0.74), 3.485 (0.89), 3.513 (1.03), 3.587 (0.58), 3.610 (0.96), 3.632 (0.46), 3.788 (1.15), 3.817 (0.98), 4.088 (0.82), 4.106 (1.32), 4.124 (2.64), 4.131 (3.96), 4.142 (3.53), 6.207 (3.89), 6.600 (5.02), 6.767 (7.93), 7.270 (1.63), 7.271 (1.66), 7.282 (1.59), 7.290 (1.66), 7.301 (1.75), 7.303 (1.75), 7.697 (1.35), 7.701 (1.78), 7.703 (1.66), 7.707 (1.47), 7.717 (1.30), 7.721 (1.47), 7.723 (1.56), 7.727 (1.27), 8.014 (3.00), 8.019 (3.05), 8.150 (3.48), 8.350 (3.05), 8.354 (3.10), 8.361 (2.81), 8.366 (2.71), 8.580 (4.44), 8.583 (5.86), 8.585 (5.43).

### Example 156

### 2-[6-amino-5-(trifluoromethyl)pyridin-3-yl]-N-[(1R)-1-(4-fluorophenyl)ethyl]-6,7-dihydrospiro[pyrazolo[5,1-c][1,4]oxazine-4,3′-pyrrolidine]-1′-carboxamide (Mixture of Stereoisomers)

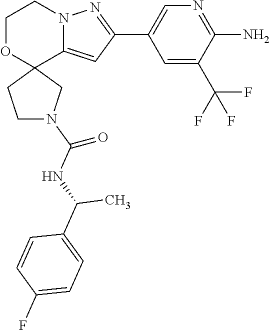 [SMILES: C[C@@H](NC(=O)N1CCC2(C1)OCCn1nc(-c3cnc(N)c(C(F)(F)F)c3)cc12)c1ccc(F)cc1]

Example 156 was prepared in analogy to Example 31 using (rac)-5-[6,7-dihydrospiro[pyrazolo[5,1-c][1,4]oxazine-4,3′-pyrrolidin]-2-yl]-3-(trifluoromethyl)pyridin-2-amine-hydrochloride salt (75.0 mg, 200 μmol) and (1R)-1-(4-fluorophenyl)ethan-1-amine (30.6 mg, 220 μmol).

1H-NMR (400 MHz, DMSO-d6) δ[ppm]: 1.003 (12.03), 1.019 (11.16), 1.349 (14.43), 1.366 (14.41), 2.074 (1.26), 2.210 (1.45), 2.236 (1.62), 2.327 (3.19), 2.523 (6.74), 2.669 (1.59), 3.105 (0.70), 3.400 (3.58), 3.449 (2.18), 3.472 (2.80), 3.500 (2.55), 3.560 (1.09), 3.582 (1.90), 3.614 (1.85), 3.638 (0.84), 3.777 (1.93), 3.804 (3.27), 3.832 (1.62), 4.073 (2.07), 4.120 (4.22), 4.134 (9.09), 4.814 (1.73), 4.833 (2.77), 4.851 (1.85), 6.494 (2.32), 6.504 (2.41), 6.514 (2.43), 6.524 (2.32), 6.599 (11.72), 6.751 (16.00), 7.090 (3.44), 7.096 (3.83), 7.112 (7.78), 7.119 (7.69), 7.134 (4.62), 7.141 (4.06), 7.351 (4.31), 7.365 (5.59), 7.369 (5.26), 7.383 (3.89), 8.010 (6.80), 8.202 (1.03), 8.580 (6.38).

### Example 157

### 2-[6-amino-5-(trifluoromethyl)pyridin-3-yl]-N-[(1R)-2,3-dihydro-1H-inden-1-yl]-6,7-dihydrospiro[pyrazolo[5,1-c][1,4]oxazine-4,3′-pyrrolidine]-1′-carboxamide (Mixture of Stereoisomers)

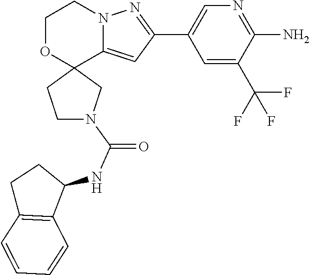 [SMILES: Nc1ncc(-c2cc3n(n2)CCOC32CCN(C(=O)N[C@@H]3CCc4ccccc43)C2)cc1C(F)(F)F]

Example 157 was prepared in analogy to Example 31 using (rac)-5-[6,7-dihydrospiro[pyrazolo[5,1-c][1,4]oxazine-4,3′-pyrrolidin]-2-yl]-3-(trifluoromethyl)pyridin-2-amine-hydrochloride salt (75.0 mg, 200 μmol) and (1R)-2,3-dihydro-1H-inden-1-amine (29.2 mg, 220 μmol).

1H-NMR (400 MHz, DMSO-d6) δ[ppm]: 1.816 (1.01), 1.840 (1.32), 1.864 (1.07), 2.074 (1.43), 2.084 (16.00), 2.232 (1.04), 2.259 (1.32), 2.282 (0.61), 2.331 (1.90), 2.347 (2.00), 2.366 (2.09), 2.378 (1.79), 2.410 (0.70), 2.669 (0.80), 2.723 (0.65), 2.745 (0.96), 2.763 (1.39), 2.784 (1.65), 2.806 (0.82), 2.875 (1.27), 2.893 (1.34), 2.909 (0.82), 2.933 (0.74), 3.388 (1.31), 3.413 (1.79), 3.431 (1.11), 3.439 (1.01), 3.472 (1.82), 3.492 (1.96), 3.501 (2.15), 3.521 (2.05), 3.589 (0.63), 3.611 (1.25), 3.640 (1.25), 3.666 (0.53), 3.819 (1.21), 3.841 (1.50), 3.868 (1.09), 4.060 (0.82), 4.074 (1.54), 4.090 (1.47), 4.108 (1.92), 4.132 (5.01), 4.143 (6.26), 5.180 (0.82), 5.201 (2.32), 5.220 (2.28), 5.241 (0.82), 6.492 (1.79), 6.499 (1.92), 6.513 (1.81), 6.520 (1.78), 6.604 (5.30), 6.760 (10.04), 7.169 (2.94), 7.174 (3.70), 7.182 (4.67), 7.191 (4.40), 7.199 (1.74), 7.214 (3.75), 7.226 (4.22), 7.234 (3.21), 8.015 (4.95), 8.020 (5.06), 8.584 (4.67).

### Example 158

### 2-[6-amino-5-(trifluoromethyl)pyridin-3-yl]-N-[(1R)-1-(5-fluoropyridin-3-yl)ethyl]-6,7-dihydrospiro[pyrazolo[5,1-c][1,4]oxazine-4,3′-pyrrolidine]-1′-carboxamide (Mixture of Stereoisomers)

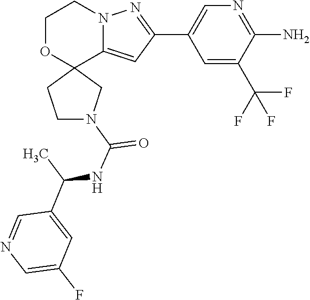 [SMILES: C[C@@H](NC(=O)N1CCC2(C1)OCCn1nc(-c3cnc(N)c(C(F)(F)F)c3)cc12)c1cncc(F)c1]

Example 158 was prepared in analogy to Example 31 using (rac)-5-[6,7-dihydrospiro[pyrazolo[5,1-c][1,4]oxazine-4,3′-pyrrolidin]-2-yl]-3-(trifluoromethyl)pyridin-2-amine-hydrochloride salt (75.0 mg, 200 μmol) and (1R)-1-(5-fluoropyridin-3-yl)ethan-1-amine (30.8 mg, 220 μmol).

¹H-NMR (400 MHz, DMSO-d₆): δ [ppm]=1.41 (d, 3H), 2.19-2.30 (m, 1H), 2.31-2.41 (m, 1H), 3.36-3.68 (m, 3H), 3.75-3.87 (m, 1H), 4.03-4.17 (m, 4H), 4.91 (td, 1H), 6.57-6.67 (m, 3H), 6.76 (d, 1H), 7.63-7.71 (m, 1H), 8.01 (s, 1H), 8.40-8.46 (m, 2H), 8.58 (s, 1H).

### Example 159

### 2-[6-amino-5-(trifluoromethyl)pyridin-3-yl]-N-[(1R)-1-(3-fluorophenyl)ethyl]-6,7-dihydrospiro[pyrazolo[5,1-c][1,4]oxazine-4,3′-pyrrolidine]-1′-carboxamide (Mixture of Stereoisomers)

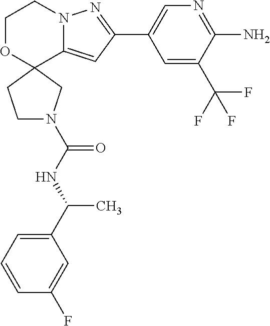 [SMILES: C[C@@H](NC(=O)N1CCC2(C1)OCCn1nc(-c3cnc(N)c(C(F)(F)F)c3)cc12)c1cccc(F)c1]

Example 159 was prepared in analogy to Example 31 using (rac)-5-[6,7-dihydrospiro[pyrazolo[5,1-c][1,4]oxazine-4,3′-pyrrolidin]-2-yl]-3-(trifluoromethyl)pyridin-2-amine-hydrochloride salt (75.0 mg, 200 μmol) and (1R)-1-(3-fluorophenyl)ethan-1-amine (30.6 mg, 220 μmol).

¹H-NMR (400 MHz, DMSO-d₆): δ [ppm]=1.36 (d, 3H), 2.17-2.29 (m, 1H), 2.31-2.40 (m, 1H), 3.36-3.53 (m, 2H), 3.55-3.67 (m, 1H), 3.77-3.87 (m, 1H), 4.01-4.18 (m, 4H), 4.79-4.90 (m, 1H), 6.54 (t, 1H), 6.60 (s, 2H), 6.76 (d, 1H), 6.97-7.05 (m, 1H), 7.12-7.20 (m, 2H), 7.34 (tdd, 1H), 8.01 (s, 1H), 8.58 (t, 1H).

### Example 160

### 2-[6-amino-5-(trifluoromethyl)pyridin-3-yl]-N-[(1R)-1-phenylpropyl]-6,7-dihydrospiro[pyrazolo[5,1-c][1,4]oxazine-4,3′-pyrrolidine]-1′-carboxamide (Mixture of Stereoisomers)

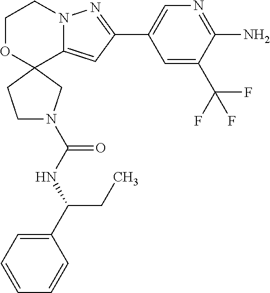 [SMILES: CC[C@@H](NC(=O)N1CCC2(C1)OCCn1nc(-c3cnc(N)c(C(F)(F)F)c3)cc12)c1ccccc1]

Example 160 was prepared in analogy to Example 31 using (rac)-5-[6,7-dihydrospiro[pyrazolo[5,1-c][1,4]oxazine-4,3′-pyrrolidin]-2-yl]-3-(trifluoromethyl)pyridin-2-amine-hydrochloride salt (75.0 mg, 200 μmol) and (1R)-1-phenylpropan-1-amine (29.7 mg, 220 μmol).

1H-NMR (400 MHz, DMSO-d6) δ[ppm]: 0.832 (5.98), 0.836 (6.36), 0.851 (14.07), 0.854 (14.27), 0.869 (7.06), 0.873 (6.82), 1.134 (0.81), 1.153 (0.50), 1.245 (0.66), 1.267 (0.69), 1.283 (0.58), 1.624 (0.89), 1.642 (1.73), 1.659 (3.05), 1.677 (3.28), 1.694 (2.58), 1.719 (2.51), 1.736 (1.93), 1.752 (1.23), 2.158 (1.08), 2.178 (2.70), 2.184 (2.39), 2.204 (2.27), 2.242 (2.39), 2.265 (1.27), 2.327 (4.20), 2.331 (3.28), 2.518 (14.77), 2.523 (9.64), 2.534 (4.74), 2.548 (2.78), 2.563 (1.20), 2.606 (0.89), 2.627 (1.16), 2.643 (0.96), 2.659 (1.31), 2.665 (1.77), 2.669 (2.27), 2.673 (1.89), 3.319 (1.23), 3.348 (2.31), 3.371 (2.47), 3.388 (2.85), 3.398 (3.32), 3.413 (3.78), 3.440 (1.81), 3.455 (2.78), 3.486 (5.24), 3.515 (4.67), 3.578 (9.91), 3.623 (3.97), 3.647 (2.04), 3.771 (2.47), 3.799 (2.27), 3.809 (2.47), 3.838 (1.93), 4.073 (2.47), 4.115 (4.32), 4.132 (10.06), 4.213 (1.31), 4.222 (1.47), 4.232 (1.93), 4.242 (1.54), 4.249 (1.50), 4.257 (1.27), 4.538 (1.39), 4.559 (3.20), 4.576 (2.97), 4.596 (1.43), 6.416 (10.56), 6.438 (3.01), 6.647 (1.89), 6.754 (14.69), 7.010 (2.04), 7.138 (2.39), 7.168 (1.70), 7.172 (1.73), 7.188 (4.32), 7.203 (3.08), 7.206 (3.20), 7.266 (4.51), 7.270 (3.82), 7.286 (8.56), 7.290 (8.02), 7.304 (9.29), 7.309 (16.00), 7.312 (15.96), 7.330 (4.36), 8.020 (7.71), 8.574 (7.75), 9.330 (0.89).

### Example 161

### (3′RS)-2-[6-amino-5-(trifluoromethyl)pyridin-3-yl]-N-[(3S)-2,3-dihydro-1-benzofuran-3-yl]-6,7-dihydrospiro[pyrazolo[5,1-c][1,4]oxazine-4,3′-pyrrolidine]-1′-carboxamide (Mixture of Stereoisomers)

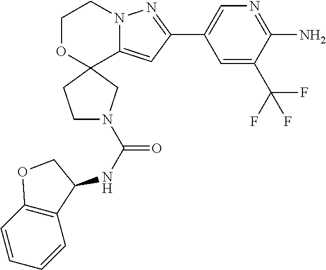 [SMILES: Nc1ncc(-c2cc3n(n2)CCOC32CCN(C(=O)N[C@@H]3COc4ccccc43)C2)cc1C(F)(F)F]

Example 161 was prepared in analogy to Example 31 using (rac)-5-[6,7-dihydrospiro[pyrazolo[5,1-c][1,4]oxazine-4,3′-pyrrolidin]-2-yl]-3-(trifluoromethyl)pyridin-2-amine-hydrochloride salt (75.0 mg, 200 μmol) and (3S)-2,3-dihydro-1-benzofuran-3-amine (29.7 mg, 220 μmol).

LC-MS (method 2): Rt=1.02 min; MS (ESIpos): m/z=501 [M+H]⁺

¹H-NMR (400 MHz, DMSO-d₆): δ [ppm]=2.15-2.28 (m, 1H), 2.30-2.39 (m, 1H), 3.38-3.42 (m, 1H), 3.47 (t, 1H), 3.54-3.66 (m, 1H), 3.82 (br t, 1H), 4.01-4.17 (m, 4H), 4.19-4.27 (m, 1H), 4.66 (t, 1H), 5.41-5.50 (m, 1H), 6.61 (br s, 2H), 6.74 (d, 1H), 6.77-6.84 (m, 2H), 6.88 (tdd, 1H), 7.15-7.22 (m, 1H), 7.32 (d, 1H), 8.01 (d, 1H), 8.58 (s, 1H).

### Example 162

### (rac)-2-[6-amino-5-(trifluoromethyl)pyridin-3-yl]-N-[(bicyclo[1.1.1]pentan-1-yl)methyl]-6,7-dihydrospiro[pyrazolo[5,1-c][1,4]oxazine-4,3′-pyrrolidine]-1′-carboxamide

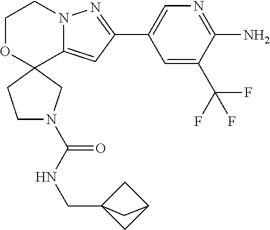 [SMILES: Nc1ncc(-c2cc3n(n2)CCOC32CCN(C(=O)NCC34CC(C3)C4)C2)cc1C(F)(F)F]

Example 162 was prepared in analogy to Example 31 using (rac)-5-[6,7-dihydrospiro[pyrazolo[5,1-c][1,4]oxazine-4,3′-pyrrolidin]-2-yl]-3-(trifluoromethyl)pyridin-2-amine-hydrochloride salt (70.0 mg, 186 μmol) and 1-(bicyclo[1.1.1]pentan-1-yl)methanamine-hydrochloride salt (27.4 mg, 205 μmol).

LC-MS (method 2): Rₜ=1.02 min; MS (ESIpos): m/z=463 [M+H]⁺

¹H-NMR (400 MHz, DMSO-d6) δ[ppm]: 1.623 (16.00), 2.435 (2.60), 2.518 (0.57), 3.057 (0.78), 3.061 (0.74), 3.075 (0.81), 3.431 (0.63), 3.460 (0.72), 3.766 (0.45), 4.117 (0.57), 4.132 (1.14), 6.113 (0.54), 6.599 (1.47), 6.741 (2.67), 8.009 (0.88), 8.014 (0.89), 8.580 (0.83), 8.585 (0.80).

### Example 163

### 2-[6-amino-5-(trifluoromethyl)pyridin-3-yl]-N-[(1S)-2-methoxy-1-phenylethyl]-6,7-dihydrospiro[pyrazolo[5,1-c][1,4]oxazine-4,3′-pyrrolidine]-1′-carboxamide (Mixture of Stereoisomers)

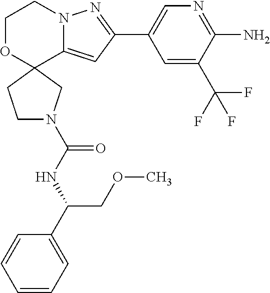 [SMILES: COC[C@@H](NC(=O)N1CCC2(C1)OCCn1nc(-c3cnc(N)c(C(F)(F)F)c3)cc12)c1ccccc1]

Example 163 was prepared in analogy to Example 31 using (rac)-5-[6,7-dihydrospiro[pyrazolo[5,1-c][1,4]oxazine-4,3′-pyrrolidin]-2-yl]-3-(trifluoromethyl)pyridin-2-amine-hydrochloride salt (70.0 mg, 186 μmol) and (1S)-2-methoxy-1-phenylethan-1-amine (31.0 mg, 205 μmol).

LC-MS (method 2): Rₜ=1.01 min; MS (ESIpos): m/z=517 [M+H]⁺

¹H-NMR (400 MHz, DMSO-d6) δ[ppm]: 2.323 (0.83), 2.327 (1.25), 2.332 (1.06), 2.337 (0.72), 2.350 (0.42), 2.518 (3.55), 2.523 (2.42), 2.665 (0.81), 2.669 (1.11), 2.674 (0.78), 3.257 (16.00), 3.291 (0.57), 3.400 (0.48), 3.429 (0.55), 3.438 (0.51), 3.443 (0.62), 3.453 (0.99), 3.463 (1.06), 3.468 (0.72), 3.478 (0.60), 3.491 (0.85), 3.520 (0.51), 3.546 (0.95), 3.567 (1.25), 3.571 (1.06), 3.592 (1.06), 3.627 (0.41), 3.786 (0.46), 3.815 (0.79), 4.074 (0.55), 4.094 (0.53), 4.111 (0.81), 4.119 (0.92), 4.124 (0.99), 4.136 (1.94), 4.945 (0.58), 4.960 (0.58), 6.495 (0.78), 6.515 (0.72), 6.599 (2.61), 6.754 (2.72), 6.758 (1.77), 7.221 (1.02), 7.237 (0.76), 7.240 (0.76), 7.285 (0.79), 7.289 (0.94), 7.300 (0.69), 7.303 (1.91), 7.308 (1.78), 7.321 (1.22), 7.326 (1.13), 7.346 (2.42), 7.362 (0.97), 7.367 (1.22), 8.009 (1.55), 8.576 (1.55).

### Example 164

### 2′-[6-amino-5-(trifluoromethyl)pyridin-3-yl]-N-[(1R)-1-(1,2-oxazol-3-yl)ethyl]-5′,6′-dihydrospiro[azetidine-3,4′-pyrrolo[1,2-b]pyrazole]-1-carboxamide

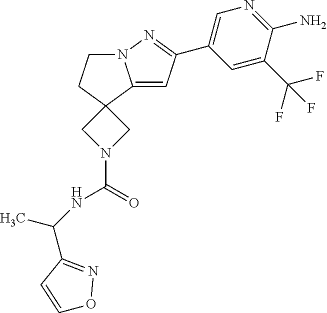 [SMILES: CC(NC(=O)N1CC2(CCn3nc(-c4cnc(N)c(C(F)(F)F)c4)cc32)C1)c1ccon1]

Example 164 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 1:1-(1,2-oxazol-3-yl)ethan-1-amine-hydrochloride salt (43.2 mg, 291 μmol).

LC-MS (method 2): Rt=0.87 min; MS (ESIpos): m/z=448 [M+H]⁺

¹H-NMR (400 MHz, DMSO-d₆): δ [ppm]=1.41 (d, 3H), 2.85 (t, 2H), 3.98-4.07 (m, 4H), 4.13 (t, 2H), 4.91-5.03 (m, 1H), 6.49-6.58 (m, 3H), 6.74 (s, 1H), 6.98 (d, 1H), 8.04 (d, 1H), 8.62 (d, 1H), 8.81 (d, 1H).

### Example 165

### 2-[6-amino-5-(trifluoromethyl)pyridin-3-yl]-N-[(1R)-1-(2-fluorophenyl)ethyl]-6,7-dihydrospiro[pyrazolo[5,1-c][1,4]oxazine-4,3′-pyrrolidine]-1′-carboxamide (Mixture of Stereoisomers)

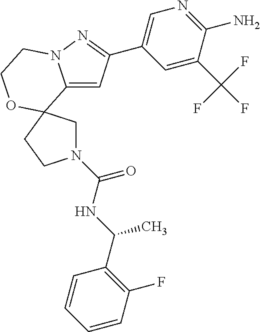 [SMILES: C[C@@H](NC(=O)N1CCC2(C1)OCCn1nc(-c3cnc(N)c(C(F)(F)F)c3)cc12)c1ccccc1F]

Example 165 was prepared in analogy to Example 31 using (rac)-5-[6,7-dihydrospiro[pyrazolo[5,1-c][1,4]oxazine-4,3′-pyrrolidin]-2-yl]-3-(trifluoromethyl)pyridin-2-amine-hydrochloride salt (100 mg, 266 μmol) and (1R)-1-(2-fluorophenyl)ethan-1-amine (44.4 mg, 319 μmol).

LC-MS (method 1): Rₜ=1.08 min; MS (ESIpos): m/z=505 [M+H]⁺

¹H-NMR (400 MHz, DMSO-d6) δ[ppm]: 0.934 (1.09), 0.951 (1.09), 1.028 (0.57), 1.035 (0.45), 1.046 (0.67), 1.237 (2.54), 1.253 (2.52), 1.345 (15.87), 1.363 (16.00), 2.198 (0.45), 2.222 (1.36), 2.247 (1.61), 2.272 (0.78), 2.327 (1.67), 2.331 (1.87), 2.349 (1.73), 2.522 (3.58), 2.664 (0.58), 2.668 (0.76), 2.673 (0.54), 3.383 (1.38), 3.394 (1.45), 3.408 (2.05), 3.423 (1.53), 3.433 (1.25), 3.449 (0.72), 3.466 (1.46), 3.494 (3.04), 3.522 (1.90), 3.577 (0.82), 3.599 (1.49), 3.620 (1.37), 3.641 (1.65), 3.664 (0.74), 3.794 (1.79), 3.822 (3.32), 3.849 (1.63), 4.049 (0.65), 4.063 (1.38), 4.079 (2.16), 4.098 (2.41), 4.115 (3.66), 4.122 (4.36), 4.128 (5.16), 4.140 (8.53), 4.239 (0.47), 4.256 (0.47), 5.068 (0.46), 5.088 (1.95), 5.106 (2.98), 5.124 (2.01), 5.142 (0.54), 6.564 (2.34), 6.576 (2.75), 6.584 (3.11), 6.602 (11.55), 6.758 (11.76), 7.089 (2.45), 7.111 (3.25), 7.137 (3.82), 7.151 (1.54), 7.156 (3.02), 7.160 (2.87), 7.169 (3.21), 7.172 (3.13), 7.178 (2.12), 7.188 (2.13), 7.191 (1.78), 7.221 (1.20), 7.226 (2.08), 7.230 (1.42), 7.234 (1.36), 7.239 (2.65), 7.244 (2.74), 7.259 (2.38), 7.263 (1.91), 7.277 (0.95), 7.422 (1.07), 7.426 (1.13), 7.441 (2.67), 7.445 (2.15), 7.454 (2.27), 7.459 (2.59), 7.474 (1.12), 7.561 (0.41), 8.008 (3.99), 8.013 (7.02), 8.018 (4.08), 8.575 (3.49), 8.582 (5.16), 8.587 (3.56).

### Example 166

### (rac)-2-[6-amino-5-(trifluoromethyl)pyridin-3-yl]-N-[2-(3-fluorophenyl)propan-2-yl]-6,7-dihydrospiro[pyrazolo[5,1-c][1,4]oxazine-4,3′-pyrrolidine]-1′-carboxamide

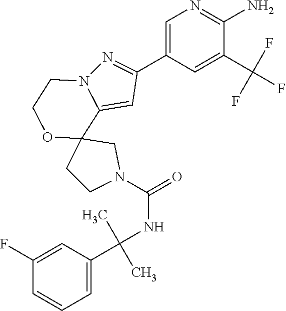 [SMILES: CC(C)(NC(=O)N1CCC2(C1)OCCn1nc(-c3cnc(N)c(C(F)(F)F)c3)cc12)c1cccc(F)c1]

Example 166 was prepared in analogy to Example 31 using (rac)-5-[6,7-dihydrospiro[pyrazolo[5,1-c][1,4]oxazine-4,3′-pyrrolidin]-2-yl]-3-(trifluoromethyl)pyridin-2-amine-hydrochloride salt (100 mg, 266 μmol) and 2-(3-fluorophenyl)propan-2-amine (48.9 mg, 319 μmol).

LC-MS (method 1): Rₜ=1.16 min; MS (ESIpos): m/z=520 [M+H]⁺

¹H-NMR (400 MHz, DMSO-d6) δ[ppm]: 0.934 (2.46), 0.951 (2.43), 1.401 (0.63), 1.425 (0.53), 1.549 (16.00), 1.555 (15.67), 2.221 (0.70), 2.247 (0.99), 2.271 (0.53), 2.318 (0.99), 2.322 (1.01), 2.327 (1.21), 2.332 (1.32), 2.348 (0.58), 2.365 (0.51), 2.518 (1.97), 2.523 (1.23), 2.669 (0.48), 3.370 (0.60), 3.394 (0.91), 3.412 (0.88), 3.437 (0.43), 3.484 (1.12), 3.513 (1.32), 3.587 (0.70), 3.608 (1.20), 3.631 (0.56), 3.790 (1.47), 3.819 (1.25), 4.076 (0.41), 4.090 (0.97), 4.107 (1.72), 4.123 (3.63), 4.131 (4.88), 4.143 (4.62), 6.152 (4.56), 6.599 (6.18), 6.765 (9.03), 6.938 (0.78), 6.943 (0.89), 6.958 (1.57), 6.965 (1.75), 6.981 (0.90), 6.985 (0.99), 7.105 (1.20), 7.110 (1.65), 7.115 (1.30), 7.133 (1.20), 7.138 (1.67), 7.143 (1.31), 7.176 (1.87), 7.197 (2.47), 7.275 (1.37), 7.290 (1.60), 7.294 (2.24), 7.310 (2.14), 7.314 (1.18), 7.330 (0.89), 8.013 (3.59), 8.019 (3.63), 8.583 (3.42), 8.587 (3.37).

### Example 167

### 2′-[6-amino-5-(trifluoromethyl)pyridin-3-yl]-N-[(1R)-1-(1-phenyl-1H-pyrazol-4-yl)ethyl]-5′,6′-dihydrospiro[azetidine-3,4′-pyrrolo[1,2-b]pyrazole]-1-carboxamide

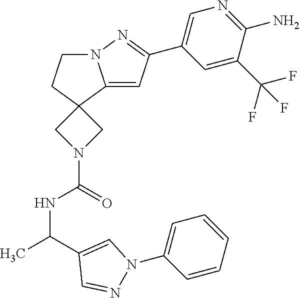 [SMILES: CC(NC(=O)N1CC2(CCn3nc(-c4cnc(N)c(C(F)(F)F)c4)cc32)C1)c1cnn(-c2ccccc2)c1]

Example 167 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 1-(1-phenyl-1H-pyrazol-4-yl)ethan-1-amine (54.5 mg, 291 μmol).

LC-MS (method 2): Rt=1.06 min; MS (ESIpos): m/z=523 [M+H]⁺

¹H-NMR (400 MHz, DMSO-d₆): δ [ppm]=1.43 (d, 3H), 2.85 (t, 2H), 3.99-4.07 (m, 4H), 4.08-4.18 (m, 2H), 4.88 (quin, 1H), 6.55 (s, 2H), 6.72-6.79 (m, 2H), 7.25-7.32 (m, 1H), 7.43-7.53 (m, 2H), 7.68 (s, 1H), 7.78-7.84 (m, 2H), 8.04 (d, 1H), 8.35 (s, 1H), 8.62 (d, 1H).

### Example 168

### 2′-[6-amino-5-(trifluoromethyl)pyridin-3-yl]-N-[(1S)-2-methoxy-1-phenylethyl]-5′,6′-dihydrospiro[azetidine-3,4′-pyrrolo[1,2-b]pyrazole]-1-carboxamide

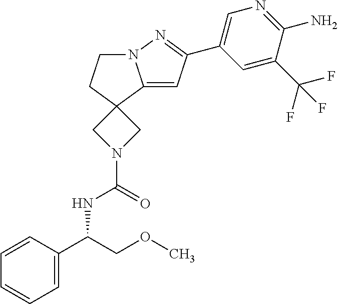 [SMILES: COC[C@@H](NC(=O)N1CC2(CCn3nc(-c4cnc(N)c(C(F)(F)F)c4)cc32)C1)c1ccccc1]

Example 168 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and (1S)-2-methoxy-1-phenylethan-1-amine (44.0 mg, 291 μmol).

LC-MS (method 2): Rt=1.03 min; MS (ESIpos): m/z=487 [M+H]⁺

¹H-NMR (400 MHz, DMSO-d₆): δ [ppm]=2.85 (t, 2H), 3.27 (s, 3H), 3.41-3.48 (m, 1H), 3.51-3.58 (m, 1H), 3.97-4.07 (m, 4H), 4.08-4.18 (m, 2H), 4.92 (td, 1H), 6.55 (s, 2H), 6.72 (s, 1H), 6.91 (d, 1H), 7.20-7.27 (m, 1H), 7.29-7.41 (m, 4H), 8.04 (d, 1H), 8.62 (d, 1H).

### Example 169

### 2′-[6-amino-5-(trifluoromethyl)pyridin-3-yl]-N-[(3-fluorobicyclo[1.1.1]pentan-1-yl)methyl]-5′,6′-dihydrospiro[azetidine-3,4′-pyrrolo[1,2-b]pyrazole]-1-carboxamide

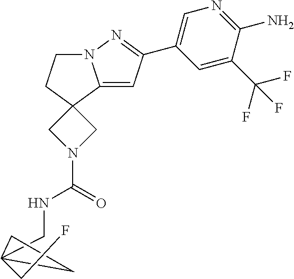 [SMILES: Nc1ncc(-c2cc3n(n2)CCC32CN(C(=O)NCC3(CF)CC3)C2)cc1C(F)(F)F]

Example 169 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 1-(3-fluorobicyclo[1.1.1]pentan-1-yl)methanamine-hydrochloride salt (44.1 mg, 291 μmol).

LC-MS (method 2): Rt=0.97 min; MS (ESIpos): m/z=451 [M+H]⁺

¹H-NMR (400 MHz, DMSO-d₆): δ [ppm]=1.86-1.94 (m, 8H), 2.85 (t, 2H), 3.95-4.03 (m, 4H), 4.12 (t, 2H), 6.55 (s, 2H), 6.62 (t, 1H), 6.72 (s, 1H), 8.04 (d, 1H), 8.62 (d, 1H).

### Example 170

### 2′-[6-amino-5-(trifluoromethyl)pyridin-3-yl]-N-[(bicyclo[1.1.1]pentan-1-yl)methyl]-5′,6′-dihydrospiro[azetidine-3,4′-pyrrolo[1,2-b]pyrazole]-1-carboxamide

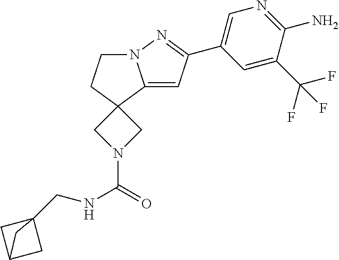 [SMILES: Nc1ncc(-c2cc3n(n2)CCC32CN(C(=O)NCC34CC(C3)C4)C2)cc1C(F)(F)F]

Example 170 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 1-(bicyclo[1.1.1]pentan-1-yl)methanamine-hydrochloride salt (38.9 mg, 291 μmol).

LC-MS (method 2): Rt=1.03 min; MS (ESIpos): m/z=433 [M+H]⁺

¹H-NMR (400 MHz, DMSO-d₆): δ [ppm]=1.63 (s, 6H), 2.45 (s, 1H), 2.84 (t, 2H), 3.04 (d, 2H), 3.94-4.02 (m, 4H), 4.12 (t, 2H), 6.47 (t, 1H), 6.54 (s, 2H), 6.72 (s, 1H), 8.04 (d, 1H), 8.62 (d, 1H).

### Example 171

### 2′-[6-amino-5-(trifluoromethyl)pyridin-3-yl]-N-[(1S)-1-(pyridin-3-yl)ethyl]-5′,6′-dihydrospiro[azetidine-3,4′-pyrrolo[1,2-b]pyrazole]-1-carboxamide

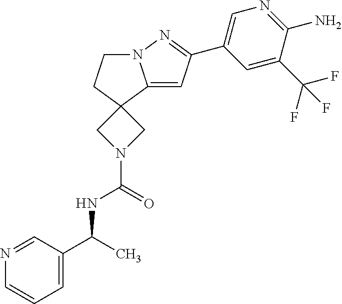 [SMILES: C[C@H](NC(=O)N1CC2(CCn3nc(-c4cnc(N)c(C(F)(F)F)c4)cc32)C1)c1cccnc1]

Example 171 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and (1S)-1-(pyridin-3-yl)ethan-1-amine-hydrochloride salt (56.8 mg, 291 μmol).

LC-MS (method 2): Rt=0.69 min; MS (ESIpos): m/z=458 [M+H]⁺

¹H-NMR (400 MHz, DMSO-d₆): δ [ppm]=1.39 (d, 3H), 2.85 (t, 2H), 3.99-4.08 (m, 4H), 4.13 (t, 2H), 4.84 (quin, 1H), 6.55 (s, 2H), 6.73 (s, 1H), 6.97 (d, 1H), 7.35 (dd, 1H), 7.74 (dt, 1H), 8.04 (d, 1H), 8.43 (dd, 1H), 8.56 (d, 1H), 8.62 (d, 1H).

### Example 172

### 2′-[6-amino-5-(trifluoromethyl)pyridin-3-yl]-N-[(1R)-1-(2-methylpyrimidin-5-yl)ethyl]-5′,6′-dihydrospiro[azetidine-3,4′-pyrrolo[1,2-b]pyrazole]-1-carboxamide

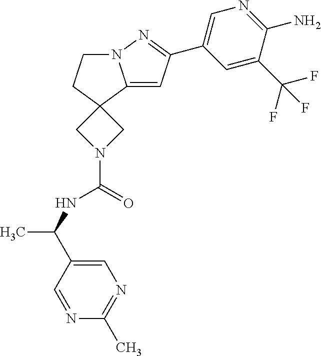 [SMILES: Cc1ncc([C@@H](C)NC(=O)N2CC3(CCn4nc(-c5cnc(N)c(C(F)(F)F)c5)cc43)C2)cn1]

Example 172 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and (1R)-1-(2-methylpyrimidin-5-yl)ethan-1-amine (39.9 mg, 291 μmol).

LC-MS (method 2): Rt=0.82 min; MS (ESIpos): m/z=473 [M+H]⁺

¹H-NMR (400 MHz, DMSO-d₆): δ [ppm]=1.41 (d, 3H), 2.59 (s, 3H), 2.85 (t, 2H), 3.97-4.07 (m, 4H), 4.09-4.16 (m, 2H), 4.81 (quin, 1H), 6.55 (s, 2H), 6.72 (s, 1H), 6.98 (d, 1H), 8.04 (d, 1H), 8.62 (d, 1H), 8.65 (s, 1H).

### Example 173

### 2′-[6-amino-5-(trifluoromethyl)pyridin-3-yl]-N-[2-(1-methyl-1H-1,2,3-triazol-4-yl)propan-2-yl]-5′,6′-dihydrospiro[azetidine-3,4′-pyrrolo[1,2-b]pyrazole]-1-carboxamide

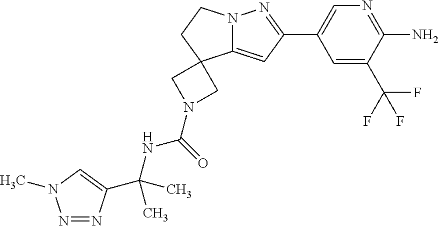 [SMILES: Cn1cc(C(C)(C)NC(=O)N2CC3(CCn4nc(-c5cnc(N)c(C(F)(F)F)c5)cc43)C2)nn1]

Example 173 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 2-(1-methyl-1H-1,2,3-triazol-4-yl)propan-2-amine-hydrochloride salt (62.0 mg, 291 μmol).

LC-MS (method 2): Rt=0.83 min; MS (ESIpos): m/z=476 [M+H]⁺

¹H-NMR (400 MHz, DMSO-d₆): δ [ppm]=1.59 (s, 6H), 2.83 (t, 2H), 3.94-4.03 (m, 7H), 4.12 (t, 2H), 6.41 (s, 1H), 6.55 (s, 2H), 6.71 (s, 1H), 7.80 (s, 1H), 8.04 (d, 1H), 8.62 (d, 1H).

### Example 174

### 2′-[6-amino-5-(trifluoromethyl)pyridin-3-yl]-N-[2-(3-chloropyridin-4-yl)propan-2-yl]-5′,6′-dihydrospiro[azetidine-3,4′-pyrrolo[1,2-b]pyrazole]-1-carboxamide

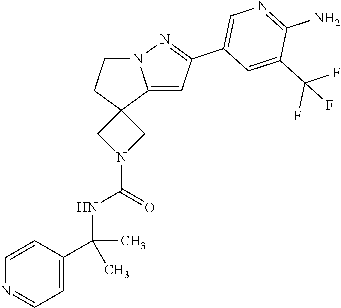 [SMILES: CC(C)(NC(=O)N1CC2(CCn3nc(-c4cnc(N)c(C(F)(F)F)c4)cc32)C1)c1ccncc1]

Example 174 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 2-(3-chloropyridin-4-yl)propan-2-amine (49.7 mg, 291 μmol).

LC-MS (method 2): Rt=0.94 min; MS (ESIpos): m/z=506 [M+H]⁺

¹H-NMR (400 MHz, DMSO-d₆): δ [ppm]=1.65 (s, 6H), 2.83 (t, 2H), 4.00 (s, 4H), 4.12 (t, 2H), 6.55 (s, 2H), 6.68 (s, 1H), 6.86 (s, 1H), 7.44 (d, 1H), 8.05 (d, 1H), 8.42 (d, 1H), 8.47 (s, 1H), 8.63 (d, 1H).

### Example 175

### 2′-[6-amino-5-(trifluoromethyl)pyridin-3-yl]-N-[1-(pyridin-4-yl)cyclobutyl]-5′,6′-dihydrospiro[azetidine-3,4′-pyrrolo[1,2-b]pyrazole]-1-carboxamide

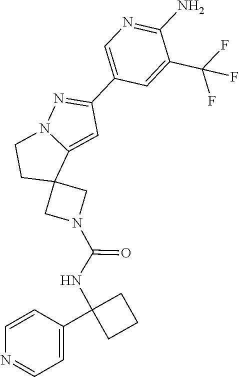 [SMILES: Nc1ncc(-c2cc3n(n2)CCC32CN(C(=O)NC3(c4ccncc4)CCC3)C2)cc1C(F)(F)F]

Example 175 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 1-(pyridin-4-yl)cyclobutan-1-amine (43.1 mg, 291 μmol).

LC-MS (method 2): Rt=0.67 min; MS (ESIpos): m/z=484 [M+H]⁺

¹H-NMR (400 MHz, DMSO-d₆): δ [ppm]=1.78-1.91 (m, 1H), 1.97-2.11 (m, 1H), 2.34-2.48 (m, 3H), 2.85 (t, 2H), 3.97-4.07 (m, 4H), 4.13 (t, 2H), 6.55 (s, 2H), 6.73 (s, 1H), 7.25 (s, 1H), 7.37-7.43 (m, 2H), 8.05 (d, 1H), 8.16 (s, 1H), 8.51 (d, 2H), 8.63 (d, 1H).

### Example 176

### 2′-[6-amino-5-(trifluoromethyl)pyridin-3-yl]-N-[(1R)-1-phenylethyl]-5′,6′-dihydrospiro[azetidine-3,4′-pyrrolo[1,2-b]pyrazole]-1-carboxamide

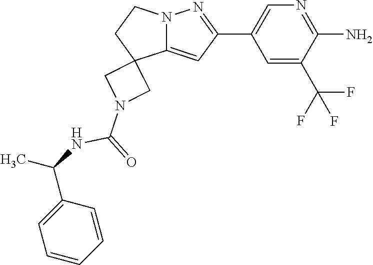 [SMILES: C[C@@H](NC(=O)N1CC2(CCn3nc(-c4cnc(N)c(C(F)(F)F)c4)cc32)C1)c1ccccc1]

Example 176 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and (1R)-1-phenylethan-1-amine (35.3 mg, 291 μmol).

LC-MS (method 2): Rt=1.04 min; MS (ESIpos): m/z=457 [M+H]⁺

¹H-NMR (400 MHz, DMSO-d₆): δ [ppm]=1.36 (d, 3H), 2.85 (t, 2H), 3.97-4.07 (m, 4H), 4.13 (t, 2H), 4.76-4.86 (m, 1H), 6.55 (s, 2H), 6.72 (s, 1H), 6.89 (d, 1H), 7.17-7.24 (m, 1H), 7.28-7.38 (m, 4H), 8.04 (d, 1H), 8.62 (d, 1H).

### Example 177

### 2-[6-amino-5-(trifluoromethyl)pyridin-3-yl]-N-[(1S)-1-(pyridin-3-yl)ethyl]-6,7-dihydrospiro[pyrazolo[5,1-c][1,4]oxazine-4,3′-pyrrolidine]-1′-carboxamide

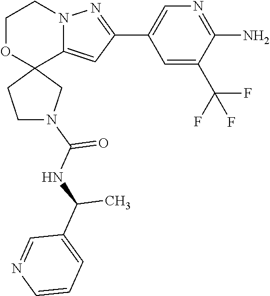 [SMILES: C[C@H](NC(=O)N1CCC2(C1)OCCn1nc(-c3cnc(N)c(C(F)(F)F)c3)cc12)c1cccnc1]

Example 177 was prepared in analogy to Example 31 using (rac)-5-[6,7-dihydrospiro[pyrazolo[5,1-c][1,4]oxazine-4,3′-pyrrolidin]-2-yl]-3-(trifluoromethyl)pyridin-2-amine-hydrochloride salt (70.0 mg, 186 μmol) and (1S)-1-(pyridin-3-yl)ethan-1-amine-hydrochloride salt (40.0 mg, 205 μmol).

LC-MS (method 2): Rₜ=0.73 min; MS (ESIpos): m/z=488 [M+H]⁺

¹H-NMR (400 MHz, DMSO-d6) δ[ppm]: 1.232 (1.55), 1.393 (16.00), 1.411 (16.00), 2.085 (0.40), 2.214 (1.26), 2.241 (1.43), 2.263 (0.63), 2.273 (0.63), 2.323 (3.10), 2.327 (4.53), 2.332 (3.73), 2.337 (2.35), 2.518 (11.35), 2.523 (7.68), 2.540 (0.63), 2.660 (1.09), 2.665 (2.52), 2.669 (3.50), 2.674 (2.47), 2.679 (1.09), 3.378 (2.92), 3.396 (2.92), 3.411 (1.84), 3.422 (1.66), 3.437 (0.92), 3.454 (1.95), 3.485 (3.15), 3.516 (1.89), 3.563 (0.86), 3.585 (1.43), 3.606 (1.43), 3.625 (1.78), 3.649 (0.80), 3.776 (1.66), 3.806 (1.55), 3.812 (2.12), 3.842 (1.66), 4.047 (0.69), 4.070 (1.61), 4.077 (1.89), 4.085 (1.89), 4.109 (3.21), 4.122 (4.42), 4.135 (8.66), 4.832 (0.40), 4.851 (1.66), 4.869 (2.29), 4.888 (1.55), 6.577 (2.81), 6.597 (13.71), 6.612 (2.75), 6.751 (8.89), 6.756 (9.35), 7.315 (1.66), 7.319 (2.12), 7.321 (2.12), 7.327 (1.84), 7.333 (3.67), 7.338 (2.35), 7.340 (2.29), 7.347 (1.95), 7.350 (2.24), 7.352 (2.24), 7.723 (1.20), 7.728 (3.27), 7.733 (3.56), 7.738 (1.72), 7.743 (1.32), 7.747 (2.92), 7.753 (3.15), 7.758 (1.38), 8.005 (4.24), 8.010 (6.94), 8.194 (4.24), 8.406 (3.10), 8.409 (5.16), 8.413 (3.90), 8.418 (3.21), 8.421 (5.05), 8.425 (3.50), 8.548 (6.65), 8.554 (6.59), 8.572 (3.73), 8.577 (6.02).

### Example 178

### (rac)-2-[6-amino-5-(trifluoromethyl)pyridin-3-yl]-N-[(3-fluorobicyclo[1.1.1]pentan-1-yl)methyl]-6,7-dihydrospiro[pyrazolo[5,1-c][1,4]oxazine-4,3′-pyrrolidine]-1′-carboxamide

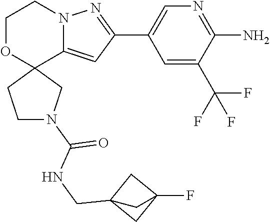 [SMILES: Nc1ncc(-c2cc3n(n2)CCOC32CCN(C(=O)NCC34CC(F)(C3)C4)C2)cc1C(F)(F)F]

Example 178 was prepared in analogy to Example 31 using (rac)-5-[6,7-dihydrospiro[pyrazolo[5,1-c][1,4]oxazine-4,3′-pyrrolidin]-2-yl]-3-(trifluoromethyl)pyridin-2-amine-hydrochloride salt (70.0 mg, 186 μmol) and 1-(3-fluorobicyclo[1.1.1]pentan-1-yl)methanamine-hydrochloride salt (31.1 mg, 205 μmol).

LC-MS (method 2): Rₜ=0.96 min; MS (ESIpos): m/z=481 [M+H]⁺

¹H-NMR (400 MHz, DMSO-d6) δ[ppm]: 1.886 (16.00), 1.892 (15.84), 2.074 (1.51), 2.214 (0.43), 2.241 (0.58), 2.322 (0.62), 2.326 (0.55), 2.337 (0.63), 2.518 (1.10), 2.522 (0.74), 3.334 (12.35), 3.355 (2.11), 3.360 (2.34), 3.379 (0.85), 3.388 (0.57), 3.431 (1.41), 3.459 (1.63), 3.528 (0.51), 3.550 (0.89), 3.761 (0.98), 3.789 (0.84), 4.061 (0.54), 4.077 (0.81), 4.096 (0.81), 4.109 (0.89), 4.117 (1.34), 4.132 (2.67), 6.266 (0.57), 6.281 (1.15), 6.295 (0.57), 6.600 (3.39), 6.746 (5.91), 8.009 (2.05), 8.014 (2.05), 8.133 (1.48), 8.580 (1.93), 8.585 (1.86).

### Example 179

### 2-[6-amino-5-(trifluoromethyl)pyridin-3-yl]-N-[(1S)-1-(pyridin-4-yl)ethyl]-6,7-dihydrospiro[pyrazolo[5,1-c][1,4]oxazine-4,3′-pyrrolidine]-1′-carboxamide (Mixture of Stereoisomers)

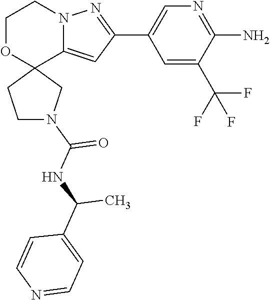 [SMILES: C[C@H](NC(=O)N1CCC2(C1)OCCn1nc(-c3cnc(N)c(C(F)(F)F)c3)cc12)c1ccncc1]

Example 179 was prepared in analogy to Example 31 using (rac)-5-[6,7-dihydrospiro[pyrazolo[5,1-c][1,4]oxazine-4,3′-pyrrolidin]-2-yl]-3-(trifluoromethyl)pyridin-2-amine-hydrochloride salt (70.0 mg, 186 μmol) and (1S)-1-(pyridin-4-yl)ethan-1-amine (25.0 mg, 205 μmol).

LC-MS (method 2): Rₜ=0.66 min; MS (ESIpos): m/z=488 [M+H]⁺

¹H-NMR (400 MHz, DMSO-d6) δ[ppm]: 0.852 (0.41), 1.232 (1.50), 1.359 (15.45), 1.378 (15.66), 2.225 (1.16), 2.252 (1.44), 2.273 (0.68), 2.318 (1.37), 2.323 (3.15), 2.327 (4.58), 2.332 (3.62), 2.337 (2.46), 2.352 (1.44), 2.386 (0.82), 2.518 (16.00), 2.523 (10.32), 2.540 (0.96), 2.660 (1.30), 2.665 (3.08), 2.669 (4.24), 2.673 (2.94), 2.679 (1.37), 3.388 (0.96), 3.414 (1.85), 3.431 (1.50), 3.466 (0.96), 3.504 (2.05), 3.534 (2.32), 3.576 (1.23), 3.596 (2.05), 3.622 (1.16), 3.649 (0.89), 3.672 (0.41), 3.788 (2.39), 3.814 (2.05), 3.828 (1.09), 3.858 (0.89), 4.053 (0.82), 4.067 (1.37), 4.084 (1.98), 4.102 (2.19), 4.118 (3.15), 4.126 (4.65), 4.131 (4.38), 4.139 (7.93), 4.789 (1.64), 4.808 (2.39), 4.826 (1.64), 6.603 (10.87), 6.625 (3.83), 6.645 (2.60), 6.760 (4.92), 6.767 (11.83), 7.321 (7.59), 7.326 (6.70), 7.332 (6.29), 7.337 (8.82), 7.343 (3.56), 8.015 (6.36), 8.021 (4.79), 8.217 (0.48), 8.472 (9.57), 8.477 (8.27), 8.483 (6.22), 8.487 (10.39), 8.492 (4.17), 8.583 (5.54), 8.589 (4.38).

### Example 180

### 2′-[6-amino-5-(trifluoromethyl)pyridin-3-yl]-N-(1-phenylcyclobutyl)-5′,6′-dihydrospiro[azetidine-3,4′-pyrrolo[1,2-b]pyrazole]-1-carboxamide

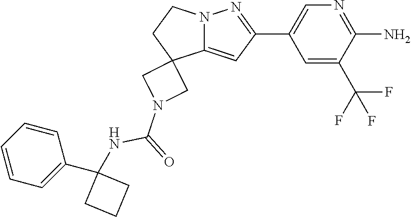 [SMILES: Nc1ncc(-c2cc3n(n2)CCC32CN(C(=O)NC3(c4ccccc4)CCC3)C2)cc1C(F)(F)F]

Example 180 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 1-phenylcyclobutan-1-amine-hydrochloride salt (49.0 mg, 267 μmol).

LC-MS (method 1): Rt=1.17 min; MS (ESIpos): m/z=483 [M+H]⁺

¹H-NMR (400 MHz, DMSO-d₆): δ [ppm]=1.78 (ddt, 1H), 1.96-2.07 (m, 1H), 2.35-2.48 (m, 4H), 2.84 (t, 2H), 3.95-4.03 (m, 4H), 4.12 (t, 2H), 6.55 (s, 2H), 6.70 (s, 1H), 7.08 (s, 1H), 7.15-7.22 (m, 1H), 7.28-7.35 (m, 2H), 7.40-7.46 (m, 2H), 8.04 (d, 1H), 8.62 (d, 1H).

### Example 181

### 2′-(6-amino-5-cyanopyridin-3-yl)-N-[1-(pyridin-4-yl)cyclobutyl]-5′,6′-dihydrospiro[azetidine-3,4′-pyrrolo[1,2-b]pyrazole]-1-carboxamide

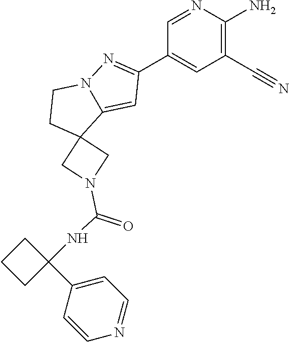 [SMILES: N#Cc1cc(-c2cc3n(n2)CCC32CN(C(=O)NC3(c4ccncc4)CCC3)C2)cnc1N]

Example 181 was prepared in analogy to Example 31 using 2-amino-5-(5′,6′-dihydrospiro[azetidine-3,4′-pyrrolo[1,2-b]pyrazol]-2′-yl)pyridine-3-carbonitrile hydrochloride (75.0 mg, 248 μmol) and 1-(pyridin-4-yl)cyclobutan-1-amine (44.1 mg, 297 μmol).

¹H NMR (400 MHz, DMSO-d₆) δ ppm 1.79-1.90 (m, 1H) 1.98-2.09 (m, 1H) 2.36-2.47 (m, 4H) 2.82-2.89 (m, 2H) 4.02 (q, 4H) 4.13 (t, 2H) 6.69 (s, 1H) 7.00 (s, 2H) 7.26 (s, 1H) 7.41 (d, 2H) 8.17 (d, 1H) 8.52 (d, 2H) 8.65 (d, 1H)

LC-MS (method 1): Rₜ=0.81 min; MS (ESIneg): m/z=439 [M−H]⁻

### Example 182

### 2′-(6-amino-5-cyanopyridin-3-yl)-N-[1-(3-fluorophenyl)cyclobutyl]-5′,6′-dihydrospiro[azetidine-3,4′-pyrrolo[1,2-b]pyrazole]-1-carboxamide

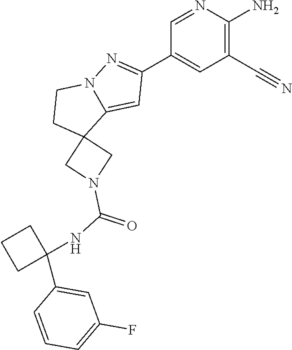 [SMILES: N#Cc1cc(-c2cc3n(n2)CCC32CN(C(=O)NC3(c4cccc(F)c4)CCC3)C2)cnc1N]

Example 182 was prepared in analogy to Example 31 using 2-amino-5-(5′,6′-dihydrospiro[azetidine-3,4′-pyrrolo[1,2-b]pyrazol]-2′-yl)pyridine-3-carbonitrile hydrochloride (75.0 mg, 248 μmol) and 1-(3-fluorophenyl)cyclobutan-1-amine hydrochloride (59.9 mg, 297 μmol).

¹H NMR (400 MHz, DMSO-d₆) δ ppm 1.74-1.85 (m, 1H) 1.96-2.09 (m, 1H) 2.36-2.48 (m, 4H) 2.84 (t, 2H) 4.00 (q, 4H) 4.12 (t, 2H) 6.67 (s, 1H) 6.96-7.05 (m, 3H) 7.12-7.16 (m, 1H) 7.19 (dt, 1H) 7.26 (d, 1H) 7.36 (td, 1H) 8.16 (d, 1H) 8.64 (d, 1H)

LC-MS (method 1): Rₜ=1.04 min; MS (ESIneg): m/z=456 [M−H]⁻

### Example 183

### 2′-(6-amino-5-cyanopyridin-3-yl)-N-[2-(3-chloropyridin-4-yl)propan-2-yl]-5′,6′-dihydrospiro[azetidine-3,4′-pyrrolo[1,2-b]pyrazole]-1-carboxamide

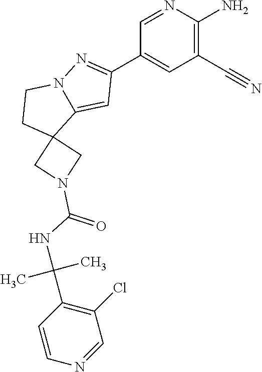 [SMILES: CC(C)(NC(=O)N1CC2(CCn3nc(-c4cnc(N)c(C#N)c4)cc32)C1)c1ccncc1Cl]

Example 183 was prepared in analogy to Example 31 using 2-amino-5-(5′,6′-dihydrospiro[azetidine-3,4′-pyrrolo[1,2-b]pyrazol]-2′-yl)pyridine-3-carbonitrile (75.0 mg, 282 μmol) and 2-(3-chloropyridin-4-yl)propan-2-amine (57.7 mg, 338 μmol).

¹H NMR (400 MHz, DMSO-d₆) δ ppm 1.64 (s, 5H) 2.52-2.56 (m, 1H) 2.78-2.89 (m, 2H) 3.99 (d, 3H) 4.07-4.21 (m, 2H) 6.60-6.72 (m, 1H) 6.87 (s, 1H) 6.94-7.09 (m, 2H) 7.44 (d, 1H) 8.18 (d, 1H) 8.43 (d, 1H) 8.48 (s, 1H) 8.65 (d, 1H)

LC-MS (method 1): Rₜ=0.88 min; MS (ESIpos): m/z=463 [M+H]⁺

### Example 184

### 2′-(6-amino-5-cyanopyridin-3-yl)-N-[(1R)-1-(5-fluoropyridin-3-yl)ethyl]-5′,6′-dihydrospiro[azetidine-3,4′-pyrrolo[1,2-b]pyrazole]-1-carboxamide

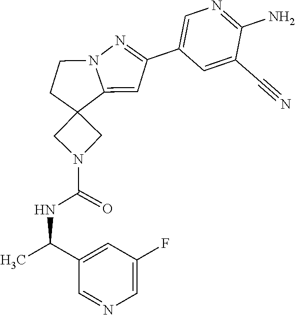 [SMILES: C[C@@H](NC(=O)N1CC2(CCn3nc(-c4cnc(N)c(C#N)c4)cc32)C1)c1cncc(F)c1]

Example 184 was prepared in analogy to Example 31 using 2-amino-5-(5′,6′-dihydrospiro[azetidine-3,4′-pyrrolo[1,2-b]pyrazol]-2′-yl)pyridine-3-carbonitrile (75.0 mg, 282 μmol) and (1R)-1-(5-fluoropyridin-3-yl)ethan-1-amine (47.4 mg, 338 μmol).

¹H NMR (400 MHz, DMSO-d₆) δ ppm 1.40 (d, 3H) 2.85 (t, 2H) 3.98-4.04 (m, 3H) 4.05-4.09 (m, 1H) 4.12 (t, 2H) 4.89 (quin, 1H) 6.68 (s, 1H) 6.99-7.01 (m, 2H) 7.66 (s, 1H) 7.68-7.70 (m, 1H) 8.15 (d, 1H) 8.43-8.47 (m, 2H) 8.63 (d, 1H)

LC-MS (method 1): Rₜ=0.81 min; MS (ESIpos): m/z=433 [M+H]⁺

### Example 185

### 2′-(6-amino-5-cyanopyridin-3-yl)-N-[(1R)-1-(4-fluorophenyl)ethyl]-5′,6′-dihydrospiro[azetidine-3,4′-pyrrolo[1,2-b]pyrazole]-1-carboxamide

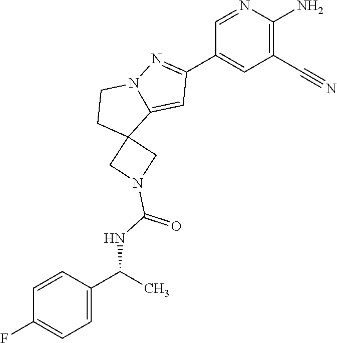 [SMILES: C[C@@H](NC(=O)N1CC2(CCn3nc(-c4cnc(N)c(C#N)c4)cc32)C1)c1ccc(F)cc1]

Example 185 was prepared in analogy to Example 31 using 2-amino-5-(5′,6′-dihydrospiro[azetidine-3,4′-pyrrolo[1,2-b]pyrazol]-2′-yl)pyridine-3-carbonitrile (75.0 mg, 282 μmol) and (1R)-1-(4-fluorophenyl)ethan-1-amine (47.0 mg, 338 μmol).

¹H NMR (400 MHz, DMSO-d₆) δ ppm 1.35 (d, 3H) 2.84 (t, 2H) 3.96-4.06 (m, 4H) 4.12 (t, 2H) 4.81 (quin, 1H) 6.68 (s, 1H) 6.91 (d, 1H) 6.95-7.07 (m, 2H) 7.13 (t, 2H) 7.36 (t, 2H) 8.15 (d, 1H) 8.64 (d, 1H)

LC-MS (method 1): Rₜ=0.97 min; MS (ESIneg): m/z=430 [M−H]⁻

### Example 186

### 2′-(6-amino-5-cyanopyridin-3-yl)-N-[2-(3-fluorophenyl)propan-2-yl]-5′,6′-dihydrospiro[azetidine-3,4′-pyrrolo[1,2-b]pyrazole]-1-carboxamide

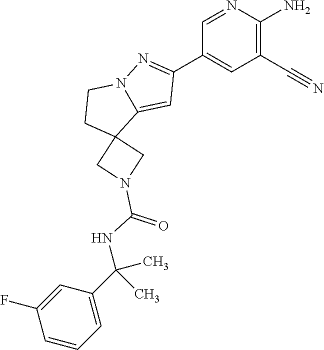 [SMILES: CC(C)(NC(=O)N1CC2(CCn3nc(-c4cnc(N)c(C#N)c4)cc32)C1)c1cccc(F)c1]

Example 186 was prepared in analogy to Example 31 using 2-amino-5-(5′,6′-dihydrospiro[azetidine-3,4′-pyrrolo[1,2-b]pyrazol]-2′-yl)pyridine-3-carbonitrile (75.0 mg, 282 μmol) and 2-(3-fluorophenyl)propan-2-amine (51.8 mg, 338 μmol).

¹H NMR (400 MHz, DMSO-d₆) δ ppm 1.55 (s, 6H) 2.81-2.89 (m, 2H) 3.97-4.07 (m, 4H) 4.13 (t, 2H) 6.63 (s, 1H) 6.67 (s, 1H) 6.95-7.04 (m, 3H) 7.13 (dt, 1H) 7.20 (d, 1H) 7.33 (td, 1H) 8.17 (d, 1H) 8.65 (d, 1H)

LC-MS (method 1): Rₜ=1.04 min; MS (ESIpos): m/z=446 [M+H]⁺

### Example 187

### 2′-(6-amino-5-cyanopyridin-3-yl)-N-[(1S)-1-(3-chloropyridin-4-yl)ethyl]-5′,6′-dihydrospiro[azetidine-3,4′-pyrrolo[1,2-b]pyrazole]-1-carboxamide

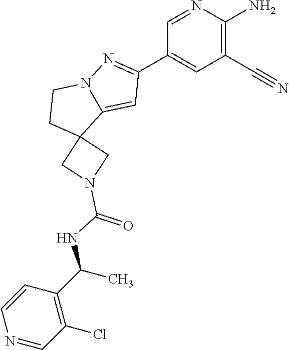 [SMILES: C[C@H](NC(=O)N1CC2(CCn3nc(-c4cnc(N)c(C#N)c4)cc32)C1)c1ccncc1Cl]

Example 187 was prepared in analogy to Example 31 using 2-amino-5-(5′,6′-dihydrospiro[azetidine-3,4′-pyrrolo[1,2-b]pyrazol]-2′-yl)pyridine-3-carbonitrile (75.0 mg, 282 μmol) and (1S)-1-(3-chloropyridin-4-yl)ethan-1-amine hydrochloride (65.3 mg, 338 μmol).

1H NMR (400 MHz, DMSO-d6) δ ppm 2.86 (t, 2H) 4.00-4.06 (m, 3H) 4.07-4.16 (m, 3H) 5.04 (quin, 1H) 6.69 (s, 1H) 6.77 (d, 1H) 7.00 (s, 2H) 7.20 (d, 1H) 7.39 (d, 1H) 7.49 (d, 1H) 8.16 (d, 1H) 8.50-8.54 (m, 2H) 8.55 (s, 1H) 8.64 (d, 1H)

LC-MS (method 1): Rₜ=0.85 min; MS (ESIpos): m/z=449 [M+H]⁺

### Example 188

### 2′-(6-amino-5-cyanopyridin-3-yl)-N-[(1-methyl-2-oxabicyclo[2.1.1]hexan-4-yl)methyl]-5′,6′-dihydrospiro[azetidine-3,4′-pyrrolo[1,2-b]pyrazole]-1-carboxamide

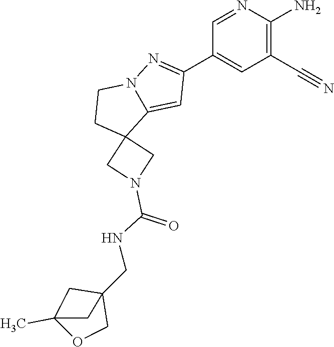 [SMILES: CC12CC(CNC(=O)N3CC4(CCn5nc(-c6cnc(N)c(C#N)c6)cc54)C3)(CO1)C2]

Example 188 was prepared in analogy to Example 31 using 2-amino-5-(5′,6′-dihydrospiro[azetidine-3,4′-pyrrolo[1,2-b]pyrazol]-2′-yl)pyridine-3-carbonitrile hydrochloride (75.0 mg, 248 μmol) and 1-(1-methyl-2-oxabicyclo[2.1.1]hexan-4-yl)methanamine (37.8 mg, 297 μmol).

¹H NMR (400 MHz, DMSO-d₆) δ ppm 1.23-1.29 (m, 3H) 1.31 (s, 3H) 1.36 (dd, 2H) 1.44-1.55 (m, 2H) 2.80-2.89 (m, 2H) 3.47 (s, 2H) 3.95-4.01 (m, 3H) 4.12 (t, 2H) 6.62 (s, 1H) 6.67 (s, 1H) 6.92-7.08 (m, 2H) 8.16 (d, 1H) 8.64 (d, 1H)

LC-MS (method 1): Rₜ=0.75 min; MS (ESIpos): m/z=420 [M+H]⁺

### Example 189

### 2′-(6-amino-5-cyanopyridin-3-yl)-N-[(1R)-1-(4-cyanopyridin-2-yl)ethyl]-5′,6′-dihydrospiro[azetidine-3,4′-pyrrolo[1,2-b]pyrazole]-1-carboxamide

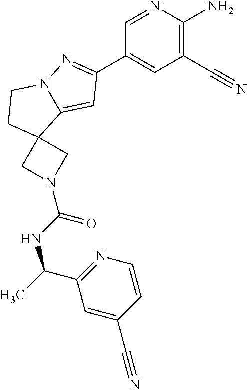 [SMILES: C[C@@H](NC(=O)N1CC2(CCn3nc(-c4cnc(N)c(C#N)c4)cc32)C1)c1cc(C#N)ccn1]

Example 189 was prepared in analogy to Example 31 using 2-amino-5-(5′,6′-dihydrospiro[azetidine-3,4′-pyrrolo[1,2-b]pyrazol]-2′-yl)pyridine-3-carbonitrile (75.0 mg, 282 μmol) and 2-[(1R)-1-aminoethyl]pyridine-4-carbonitrile hydrochloride (62.1 mg, 338 μmol).

¹H NMR (400 MHz, DMSO-d₆) δ ppm 1.41 (d, 3H) 2.85 (t, 2H) 3.98-4.06 (m, 3H) 4.06-4.16 (m, 3H) 4.90 (quin, 1H) 6.68 (s, 1H) 6.96-7.05 (m, 3H) 8.15 (d, 1H) 8.26 (t, 1H) 8.63 (d, 1H) 8.85 (d, 1H) 8.91 (d, 1H)

LC-MS (method 1): Rₜ=0.79 min; MS (ESIpos): m/z=440 [M+H]⁺

### Example 190

### 2′-(6-amino-5-cyanopyridin-3-yl)-N-[(1R)-1-(2-cyanopyridin-4-yl)ethyl]-5′,6′-dihydrospiro[azetidine-3,4′-pyrrolo[1,2-b]pyrazole]-1-carboxamide

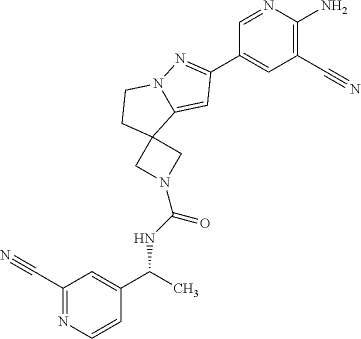 [SMILES: C[C@@H](NC(=O)N1CC2(CCn3nc(-c4cnc(N)c(C#N)c4)cc32)C1)c1ccnc(C#N)c1]

Example 190 was prepared in analogy to Example 31 using 2-amino-5-(5′,6′-dihydrospiro[azetidine-3,4′-pyrrolo[1,2-b]pyrazol]-2′-yl)pyridine-3-carbonitrile (75.0 mg, 282 μmol) and 4-[(1R)-1-aminoethyl]pyridine-2-carbonitrile (49.7 mg, 338 μmol).

¹H NMR (400 MHz, DMSO-d₆) δ ppm 1.41 (d, 3H) 2.84 (t, 2H) 3.98-4.06 (m, 3H) 4.06-4.17 (m, 3H) 4.89 (quin, 1H) 6.68 (s, 1H) 7.00 (s, 2H) 7.15 (d, 1H) 8.15 (d, 1H) 8.29 (t, 1H) 8.63 (d, 1H) 8.86 (d, 1H) 8.90 (d, 1H)

LC-MS (Method 1): Rt=0.79 min; MS (ESIpos): m/z=440 [M+H]⁺

### Example 191

### 2′-(6-amino-5-cyanopyridin-3-yl)-N-[(1R)-1-(2-chloro-4-cyanophenyl)ethyl]-5′,6′-dihydrospiro[azetidine-3,4′-pyrrolo[1,2-b]pyrazole]-1-carboxamide

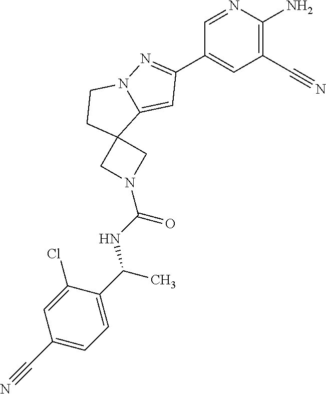 [SMILES: C[C@@H](NC(=O)N1CC2(CCn3nc(-c4cnc(N)c(C#N)c4)cc32)C1)c1ccc(C#N)cc1Cl]

Example 191 was prepared in analogy to Example 31 using 2-amino-5-(5′,6′-dihydrospiro[azetidine-3,4′-pyrrolo[1,2-b]pyrazol]-2′-yl)pyridine-3-carbonitrile (75.0 mg, 282 μmol) and 4-[(1R)-1-aminoethyl]-3-chlorobenzonitrile (61.0 mg, 338 μmol).

¹H NMR (400 MHz, DMSO-d₆) δ ppm 1.33 (d, 3H) 2.85 (t, 2H) 3.99-4.05 (m, 2H) 4.05-4.19 (m, 3H) 5.11 (quin, 1H) 6.54 (s, 1H) 6.69 (s, 1H) 7.00 (s, 2H) 7.20 (d, 1H) 7.66 (d, 1H) 7.87 (dd, 1H) 7.94-8.03 (m, 1H) 8.13-8.19 (m, 1H) 8.61-8.67 (m, 1H)

LC-MS (method 1): Rₜ=1.00 min; MS (ESIpos): m/z=473 [M+H]⁺

### Example 192

### 2′-(6-amino-5-cyanopyridin-3-yl)-N-[1-(2-fluorophenyl)cyclobutyl]-5′,6′-dihydrospiro[azetidine-3,4′-pyrrolo[1,2-b]pyrazole]-1-carboxamide

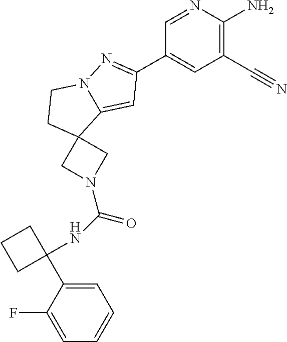 [SMILES: N#Cc1cc(-c2cc3n(n2)CCC32CN(C(=O)NC3(c4ccccc4F)CCC3)C2)cnc1N]

Example 192 was prepared in analogy to Example 31 using 2-amino-5-(5′,6′-dihydrospiro[azetidine-3,4′-pyrrolo[1,2-b]pyrazol]-2′-yl)pyridine-3-carbonitrile (75.0 mg, 282 μmol) and 1-(2-fluorophenyl)cyclobutan-1-amine hydrochloride (68.2 mg, 338 μmol).

¹H NMR (400 MHz, DMSO-d₆) δ ppm 1.75 (dt, 1H) 1.99-2.11 (m, 1H) 2.81 (t, 2H) 3.89-4.02 (m, 4H) 4.10 (t, 2H) 6.63 (s, 1H) 6.99 (s, 2H) 7.04-7.14 (m, 3H) 7.18-7.31 (m, 1H) 7.39-7.47 (m, 1H) 8.14 (d, 1H) 8.63 (d, 1H)

LC-MS (method 1): Rₜ=1.06 min; MS (ESIneg): m/z=456 [M−H]⁻

### Example 193

### 2′-(6-amino-5-cyanopyridin-3-yl)-N-[2-(4-fluorophenyl)propan-2-yl]-5′,6′-dihydrospiro[azetidine-3,4′-pyrrolo[1,2-b]pyrazole]-1-carboxamide

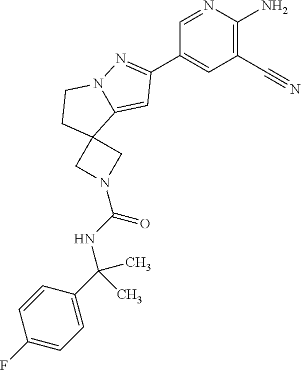 [SMILES: CC(C)(NC(=O)N1CC2(CCn3nc(-c4cnc(N)c(C#N)c4)cc32)C1)c1ccc(F)cc1]

Example 193 was prepared in analogy to Example 31 using 2-amino-5-(5′,6′-dihydrospiro[azetidine-3,4′-pyrrolo[1,2-b]pyrazol]-2′-yl)pyridine-3-carbonitrile (75.0 mg, 282 μmol) and 2-(4-fluorophenyl)propan-2-amine (51.8 mg, 338 μmol).

¹H NMR (400 MHz, DMSO-d₆) δ ppm 1.49-1.59 (m, 6H) 2.80-2.89 (m, 2H) 3.94-4.05 (m, 4H) 4.13 (t, 2H) 6.58 (s, 1H) 6.67 (s, 1H) 6.98-7.13 (m, 4H) 7.28-7.40 (m, 2H) 8.17 (d, 1H) 8.65 (d, 1H)

LC-MS (method 1): Rₜ=1.03 min; MS (ESIneg): m/z=444 [M−H]⁻

### Example 194

### 2′-(6-amino-5-cyanopyridin-3-yl)-N-[1-(2-chlorophenyl)cyclobutyl]-5′,6′-dihydrospiro[azetidine-3,4′-pyrrolo[1,2-b]pyrazole]-1-carboxamide

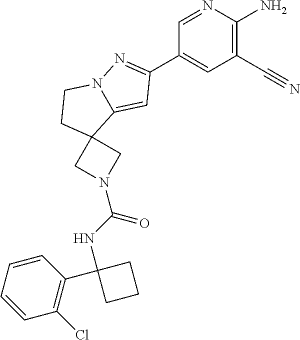 [SMILES: N#Cc1cc(-c2cc3n(n2)CCC32CN(C(=O)NC3(c4ccccc4Cl)CCC3)C2)cnc1N]

Example 194 was prepared in analogy to Example 31 using 2-amino-5-(5′,6′-dihydrospiro[azetidine-3,4′-pyrrolo[1,2-b]pyrazol]-2′-yl)pyridine-3-carbonitrile (75.0 mg, 282 μmol) and 1-(2-chlorophenyl)cyclobutan-1-amine hydrochloride (73.7 mg, 338 μmol).

¹H NMR (400 MHz, DMSO-d₆) δ ppm 1.64-1.75 (m, 1H) 2.00-2.12 (m, 1H) 2.52-2.56 (m, 1H) 2.57-2.68 (m, 2H) 2.79 (t, 2H) 3.90-4.00 (m, 4H) 4.10 (t, 2H) 6.60 (s, 1H) 6.99 (s, 2H) 7.03 (s, 1H) 7.19-7.29 (m, 2H) 7.33 (dd, 1H) 7.54 (dd, 1H) 8.14 (d, 1H) 8.62 (d, 1H)

LC-MS (method 1): Rₜ=1.12 min; MS (ESIneg): m/z=472 [M−H]⁻

### Example 195

### 2′-(6-amino-5-cyanopyridin-3-yl)-N-[2-(1-methyl-1H-1,2,3-triazol-4-yl)propan-2-yl]-5′,6′-dihydrospiro[azetidine-3,4′-pyrrolo[1,2-b]pyrazole]-1-carboxamide

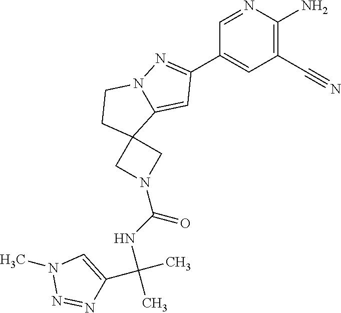 [SMILES: Cn1cc(C(C)(C)NC(=O)N2CC3(CCn4nc(-c5cnc(N)c(C#N)c5)cc43)C2)nn1]

Example 195 was prepared in analogy to Example 31 using 2-amino-5-(5′,6′-dihydrospiro[azetidine-3,4′-pyrrolo[1,2-b]pyrazol]-2′-yl)pyridine-3-carbonitrile (75.0 mg, 282 μmol) and 2-(1-methyl-1H-1,2,3-triazol-4-yl)propan-2-amine hydrochloride (72.0 mg, 338 μmol).

¹H NMR (400 MHz, DMSO-d₆) δ ppm 1.59 (s, 6H) 2.82 (t, 2H) 3.95-4.03 (m, 7H) 4.12 (t, 2H) 6.42 (s, 1H) 6.67 (s, 1H) 7.00 (s, 2H) 7.80 (s, 1H) 8.16 (d, 1H) 8.64 (d, 1H)

LC-MS (method 1): Rₜ=0.73 min; MS (ESIpos): m/z=433 [M+H]⁺

### Example 196

### 2′-(6-amino-5-cyanopyridin-3-yl)-N-[2-(pyridin-2-yl)propan-2-yl]-5′,6′-dihydrospiro[azetidine-3,4′-pyrrolo[1,2-b]pyrazole]-1-carboxamide

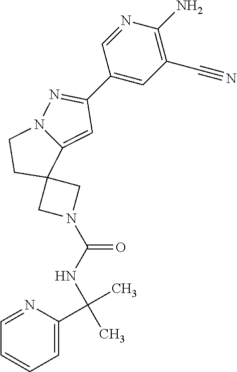 [SMILES: CC(C)(NC(=O)N1CC2(CCn3nc(-c4cnc(N)c(C#N)c4)cc32)C1)c1ccccn1]

Example 196 was prepared in analogy to Example 31 using 2-amino-5-(5′,6′-dihydrospiro[azetidine-3,4′-pyrrolo[1,2-b]pyrazol]-2′-yl)pyridine-3-carbonitrile (75.0 mg, 282 μmol) and 2-(pyridin-2-yl)propan-2-amine (46.0 mg, 338 μmol).

¹H NMR (400 MHz, DMSO-d₆) δ ppm 1.57 (s, 6H) 2.86 (t, 2H) 3.99-4.08 (m, 4H) 4.13 (t, 2H) 6.70 (s, 1H) 6.73 (s, 1H) 7.00 (s, 2H) 7.20 (ddd, 1H) 7.45 (d, 1H) 7.74 (td, 1H) 8.18 (d, 1H) 8.47 (d, 1H) 8.65 (d, 1H)

LC-MS (method 1): Rₜ=0.86 min; MS (ESIpos): m/z=429 [M+H]⁺

### Example 197

### 2′-(6-amino-5-cyanopyridin-3-yl)-N-[2-(pyridin-4-yl)propan-2-yl]-5′,6′-dihydrospiro[azetidine-3,4′-pyrrolo[1,2-b]pyrazole]-1-carboxamide

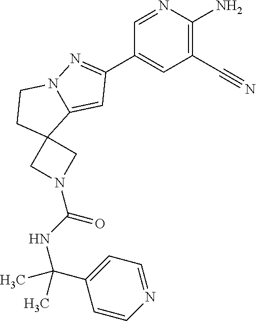 [SMILES: CC(C)(NC(=O)N1CC2(CCn3nc(-c4cnc(N)c(C#N)c4)cc32)C1)c1ccncc1]

Example 197 was prepared in analogy to Example 31 using 2-amino-5-(5′,6′-dihydrospiro[azetidine-3,4′-pyrrolo[1,2-b]pyrazol]-2′-yl)pyridine-3-carbonitrile (75.0 mg, 282 μmol) and 2-(pyridin-4-yl)propan-2-amine (46.0 mg, 338 μmol).

¹H NMR (400 MHz, DMSO-d₆) δ ppm 1.53 (s, 6H) 2.80-2.89 (m, 2H) 3.98-4.07 (m, 4H) 4.13 (t, 2H) 6.68 (s, 1H) 6.73 (s, 1H) 7.00 (s, 2H) 7.29-7.34 (m, 2H) 8.18 (d, 1H) 8.44-8.52 (m, 2H) 8.65 (d, 1H)

LC-MS (method 1): Rₜ=0.81 min; MS (ESIpos): m/z=429 [M+H]⁺

### Example 198

### 2′-(6-amino-5-cyanopyridin-3-yl)-N-[(1R)-1-phenylpropyl]-5′,6′-dihydrospiro[azetidine-3,4′-pyrrolo[1,2-b]pyrazole]-1-carboxamide

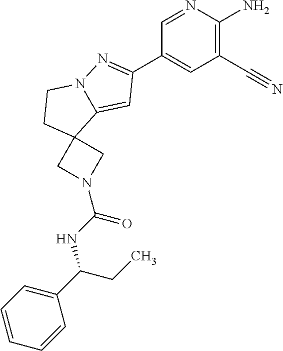 [SMILES: CC[C@@H](NC(=O)N1CC2(CCn3nc(-c4cnc(N)c(C#N)c4)cc32)C1)c1ccccc1]

Example 198 was prepared in analogy to Example 31 using 2-amino-5-(5′,6′-dihydrospiro[azetidine-3,4′-pyrrolo[1,2-b]pyrazol]-2′-yl)pyridine-3-carbonitrile (75.0 mg, 282 μmol) and (1R)-1-phenylpropan-1-amine (45.7 mg, 338 μmol).

¹H NMR (400 MHz, DMSO-d₆) δ ppm 0.86 (t, 3H) 1.58-1.75 (m, 2H) 2.84 (t, 2H) 3.96-4.06 (m, 4H) 4.12 (t, 2H) 4.48-4.58 (m, 1H) 6.67 (s, 1H) 6.83 (d, 1H) 6.99 (s, 2H) 7.17-7.25 (m, 1H) 7.28-7.35 (m, 4H) 8.15 (d, 1H) 8.63 (d, 1H)

LC-MS (method 1): Rₜ=1.02 min; MS (ESIpos): m/z=428 [M+H]⁺

### Example 199

### 2′-(6-amino-5-cyanopyridin-3-yl)-N-[(1R)-1-(3-fluorophenyl)ethyl]-5′,6′-dihydrospiro[azetidine-3,4′-pyrrolo[1,2-b]pyrazole]-1-carboxamide

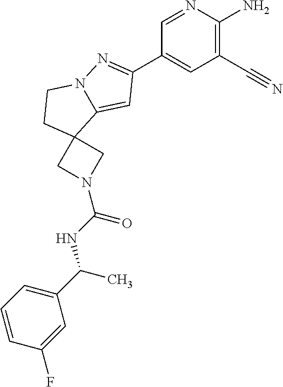 [SMILES: C[C@@H](NC(=O)N1CC2(CCn3nc(-c4cnc(N)c(C#N)c4)cc32)C1)c1cccc(F)c1]

Example 199 was prepared in analogy to Example 31 using 2-amino-5-(5′,6′-dihydrospiro[azetidine-3,4′-pyrrolo[1,2-b]pyrazol]-2′-yl)pyridine-3-carbonitrile (75.0 mg, 282 μmol) and (1R)-1-(3-fluorophenyl)ethan-1-amine (47.0 mg, 338 μmol).

¹H NMR (400 MHz, DMSO-d₆) δ ppm 1.36 (d, 3H) 2.85 (t, 2H) 3.98-4.08 (m, 4H) 4.12 (t, 2H) 4.78-4.86 (m, 1H) 6.68 (s, 1H) 6.93 (d, 1H) 7.00 (s, 2H) 7.01-7.07 (m, 1H) 7.13-7.20 (m, 2H) 7.31-7.39 (m, 1H) 8.15 (d, 1H) 8.64 (d, 1H)

LC-MS (method 1): Rₜ=0.98 min; MS (ESIpos): m/z=432 [M+H]⁺

### Example 200

### 2′-(6-amino-5-cyanopyridin-3-yl)-N-[(1R)-1-(2-cyanophenyl)ethyl]-5′,6′-dihydrospiro[azetidine-3,4′-pyrrolo[1,2-b]pyrazole]-1-carboxamide

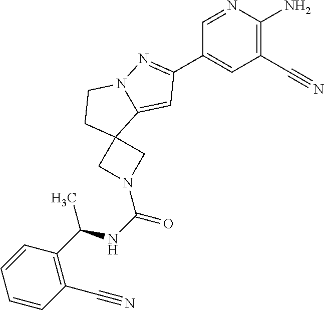 [SMILES: C[C@@H](NC(=O)N1CC2(CCn3nc(-c4cnc(N)c(C#N)c4)cc32)C1)c1ccccc1C#N]

Example 200 was prepared in analogy to Example 31 using 2-amino-5-(5′,6′-dihydrospiro[azetidine-3,4′-pyrrolo[1,2-b]pyrazol]-2′-yl)pyridine-3-carbonitrile (75.0 mg, 282 μmol) and 2-[(1R)-1-aminoethyl]benzonitrile (49.4 mg, 338 μmol).

¹H NMR (400 MHz, DMSO-d₆) δ ppm 1.39 (d, 3H) 2.81-2.89 (m, 2H) 3.99-4.10 (m, 4H) 4.13 (t, 2H) 5.03 (quin, 1H) 6.67 (s, 1H) 7.00 (s, 2H) 7.19 (d, 1H) 7.42 (td, 1H) 7.62 (d, 1H) 7.69-7.79 (m, 2H) 8.15 (d, 1H) 8.64 (d, 1H)

LC-MS (method 1): Rₜ=0.91 min; MS (ESIpos): m/z=439 [M+H]⁺

### Example 201

### (rac)-2′-(6-amino-5-cyanopyridin-3-yl)-N-[2-(pyridin-4-yl)propan-2-yl]-5′,6′-dihydrospiro[pyrrolidine-3,4′-pyrrolo[1,2-b]pyrazole]-1-carboxamide

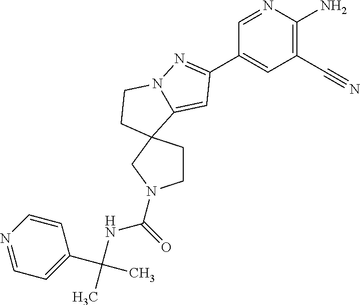 [SMILES: CC(C)(NC(=O)N1CCC2(CCn3nc(-c4cnc(N)c(C#N)c4)cc32)C1)c1ccncc1]

Example 201 was prepared in analogy to Example 31 using (rac)-2-amino-5-[5′,6′-dihydrospiro[pyrrolidine-3,4′-pyrrolo[1,2-b]pyrazol]-2′-yl]pyridine-3-carbonitrile hydrochloride (125 mg, 395 μmol) and 2-(pyridin-4-yl)propan-2-amine (64.5 mg, 473 μmol).

¹H NMR (400 MHz, DMSO-d₆) δ ppm 1.55 (s, 6H) 1.98-2.15 (m, 2H) 2.52-2.59 (m, 2H) 3.45 (br s, 2H) 3.49-3.60 (m, 2H) 4.19 (t, 2H) 6.33 (s, 1H) 6.46 (s, 1H) 7.01 (s, 2H) 7.49 (d, 2H) 8.15 (d, 1H) 8.52 (d, 2H) 8.62 (d, 1H)

LC-MS (method 1): Rₜ=0.83 min; MS (ESIpos): m/z=444 [M+H]⁺

### Example 202

### 2′-(6-amino-5-cyanopyridin-3-yl)-N-[(1R)-1-(5-fluoropyridin-3-yl)ethyl]-5′,6′-dihydrospiro[pyrrolidine-3,4′-pyrrolo[1,2-b]pyrazole]-1-carboxamide (Mixture of Stereoisomers)

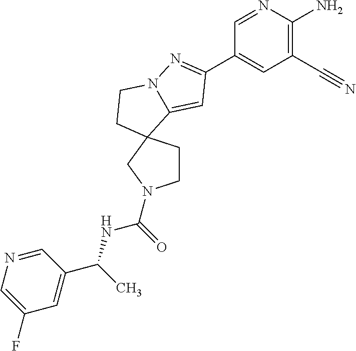 [SMILES: C[C@@H](NC(=O)N1CCC2(CCn3nc(-c4cnc(N)c(C#N)c4)cc32)C1)c1cncc(F)c1]

Example 202 was prepared in analogy to Example 31 using (rac)-2-amino-5-[5′,6′-dihydrospiro[pyrrolidine-3,4′-pyrrolo[1,2-b]pyrazol]-2′-yl]pyridine-3-carbonitrile hydrochloride (125 mg, 395 μmol) and (1R)-1-(5-fluoropyridin-3-yl)ethan-1-amine (66.4 mg, 473 μmol).

¹H NMR (400 MHz, DMSO-d₆) δ ppm 1.41 (dd, 3H) 2.01-2.12 (m, 2H) 2.52-2.57 (m, 2H) 3.46 (br dd, 3H) 3.50-3.63 (m, 1H) 4.15-4.22 (m, 2H) 4.92 (td, 1H) 6.40-6.45 (m, 1H) 6.61 (dd, 1H) 6.99 (s, 2H) 7.64-7.72 (m, 1H) 8.14 (dd, 1H) 8.40-8.47 (m, 2H) 8.61 (d, 1H)

LC-MS (method 1): Rₜ=0.83 min; MS (ESIpos): m/z=448 [M+H]⁺

### Example 203

### (rac)-2′-(6-amino-5-cyanopyridin-3-yl)-N-[2-(1-methyl-1H-1,2,3-triazol-4-yl)propan-2-yl]-5′,6′-dihydrospiro[pyrrolidine-3,4′-pyrrolo[1,2-b]pyrazole]-1-carboxamide

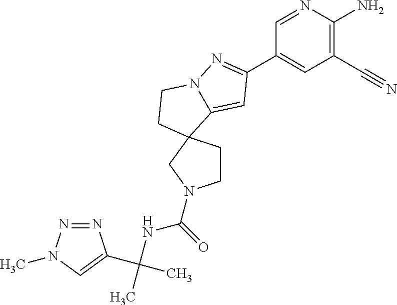 [SMILES: Cn1cc(C(C)(C)NC(=O)N2CCC3(CCn4nc(-c5cnc(N)c(C#N)c5)cc43)C2)nn1]

Example 203 was prepared in analogy to Example 31 using (rac)-2-amino-5-[5′,6′-dihydrospiro[pyrrolidine-3,4′-pyrrolo[1,2-b]pyrazol]-2′-yl]pyridine-3-carbonitrile hydrochloride (125 mg, 395 μmol) and 2-(1-methyl-1H-1,2,3-triazol-4-yl)propan-2-amine hydrochloride (101 mg, 473 μmol).

¹H NMR (400 MHz, DMSO-d₆) δ ppm 1.60 (d, 6H) 2.00-2.10 (m, 2H) 2.52-2.56 (m, 2H) 3.44 (br d, 3H) 3.51 (br s, 1H) 3.98 (s, 3H) 4.18 (t, 2H) 5.93 (s, 1H) 6.46 (s, 1H) 6.99 (s, 2H) 7.80 (s, 1H) 8.14-8.19 (m, 1H) 8.62 (d, 1H)

LC-MS (method 1): Rₜ=0.74 min; MS (ESIpos): m/z=448 [M+H]⁺

### Example 204

### (rac)-2-(6-amino-5-cyanopyridin-3-yl)-N-[1-(pyridin-2-yl)cyclobutyl]-6,7-dihydrospiro[pyrazolo[5,1-c][1,4]oxazine-4,3′-pyrrolidine]-1′-carboxamide

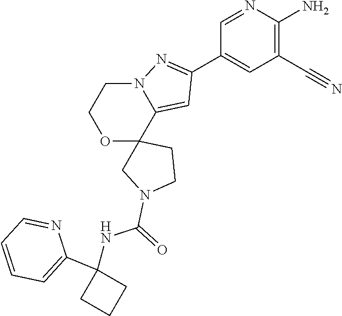 [SMILES: N#Cc1cc(-c2cc3n(n2)CCOC32CCN(C(=O)NC3(c4ccccn4)CCC3)C2)cnc1N]

Example 204 was prepared in analogy to Example 31 using (rac)-2-amino-5-[6,7-dihydrospiro[pyrazolo[5,1-c][1,4]oxazine-4,3′-pyrrolidin]-2-yl]pyridine-3-carbonitrile hydrochloride (71.0 mg, 213 μmol) and 1-(pyridin-2-yl)cyclobutan-1-amine (37.9 mg, 256 μmol).

¹H NMR (400 MHz, DMSO-d₆) δ ppm 1.83-2.08 (m, 2H) 2.18-2.31 (m, 1H) 2.32-2.43 (m, 3H) 2.53-2.65 (m, 2H) 3.35-3.53 (m, 2H) 3.64 (br t, 1H) 3.83 (br d, 1H) 4.06-4.18 (m, 4H) 6.73 (s, 1H) 6.82 (s, 1H) 7.05 (s, 2H) 7.19 (ddd, 1H) 7.36 (d, 1H) 7.66-7.72 (m, 1H) 8.12 (d, 1H) 8.53 (d, 1H) 8.61 (d, 1H)

LC-MS (method 1): Rₜ=0.90 min; MS (ESIpos): m/z=471 [M+H]⁺

### Example 205

### (rac)-2′-(6-amino-5-cyanopyridin-3-yl)-N-[1-(pyridin-2-yl)cyclobutyl]-5′,6′-dihydrospiro[pyrrolidine-3,4′-pyrrolo[1,2-b]pyrazole]-1-carboxamide

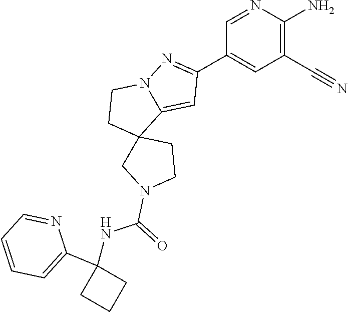 [SMILES: N#Cc1cc(-c2cc3n(n2)CCC32CCN(C(=O)NC3(c4ccccn4)CCC3)C2)cnc1N]

Example 205 was prepared in analogy to Example 31 using (rac)-2-amino-5-[5′,6′-dihydrospiro[pyrrolidine-3,4′-pyrrolo[1,2-b]pyrazol]-2′-yl]pyridine-3-carbonitrile hydrochloride (77.0 mg, 243 μmol) and 1-(pyridin-2-yl)cyclobutan-1-amine (43.2 mg, 292 μmol).

¹H NMR (400 MHz, DMSO-d₆) δ ppm 1.83-2.04 (m, 2H) 2.05-2.14 (m, 2H) 2.53-2.63 (m, 4H) 3.43-3.51 (m, 3H) 3.52-3.64 (m, 1H) 4.20 (t, 3H) 6.46 (s, 1H) 6.79 (s, 1H) 7.00 (s, 3H) 7.18 (ddd, 1H) 7.37 (d, 1H) 7.68 (td, 1H) 8.14 (d, 1H) 8.53 (d, 1H) 8.62 (d, 1H)

LC-MS (method 1): Rₜ=0.90 min; MS (ESIpos): m/z=455 [M+H]⁺

### Example 206

### (rac)-2′-(6-amino-5-cyanopyridin-3-yl)-N-[1-(2-chlorophenyl)cyclobutyl]-5′,6′-dihydrospiro[pyrrolidine-3,4′-pyrrolo[1,2-b]pyrazole]-1-carboxamide

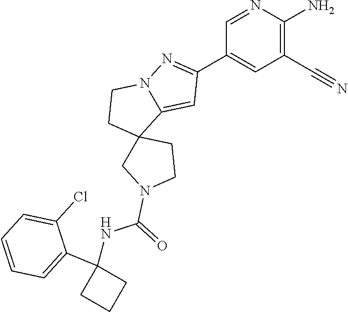 [SMILES: N#Cc1cc(-c2cc3n(n2)CCC32CCN(C(=O)NC3(c4ccccc4Cl)CCC3)C2)cnc1N]

Example 206 was prepared in analogy to Example 31 using (rac)-2-amino-5-[5′,6′-dihydrospiro[pyrrolidine-3,4′-pyrrolo[1,2-b]pyrazol]-2′-yl]pyridine-3-carbonitrile hydrochloride (77.0 mg, 243 μmol) and 1-(2-chlorophenyl)cyclobutan-1-amine hydrochloride (63.6 mg, 292 μmol).

¹H NMR (400 MHz, DMSO-d₆) δ ppm 1.63-1.73 (m, 1H) 1.98-2.14 (m, 3H) 2.38-2.47 (m, 1H) 2.52-2.70 (m, 4H) 3.38 (br d, 3H) 3.43-3.51 (m, 1H) 4.16 (t, 2H) 6.31 (s, 1H) 6.52 (s, 1H) 7.00 (s, 2H) 7.17-7.32 (m, 4H) 7.54 (dd, 1H) 8.08 (d, 1H) 8.57 (d, 1H)

LC-MS (method 1): Rₜ=1.14 min; MS (ESIpos): m/z=488 [M+H]⁺

### Example 207

### 2-(6-amino-5-cyanopyridin-3-yl)-N-[(1R)-1-phenylethyl]-6,7-dihydrospiro[pyrazolo[5,1-c][1,4]oxazine-4,3′-pyrrolidine]-1′-carboxamide (Mixture of Stereoisomers)

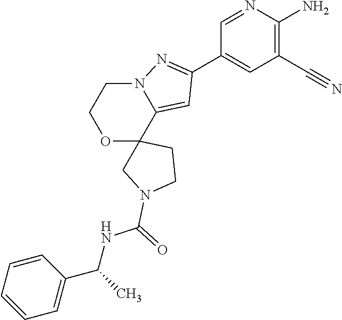 [SMILES: C[C@@H](NC(=O)N1CCC2(C1)OCCn1nc(-c3cnc(N)c(C#N)c3)cc12)c1ccccc1]

Example 207 was prepared in analogy to Example 31 using (rac)-2-amino-5-[6,7-dihydrospiro[pyrazolo[5,1-c][1,4]oxazine-4,3′-pyrrolidin]-2-yl]pyridine-3-carbonitrile hydrochloride (71.0 mg, 213 μmol) and (1R)-1-phenylethan-1-amine (31.0 mg, 256 μmol).

¹H NMR (400 MHz, DMSO-d₆) δ ppm 1.37 (d, 3H) 2.15-2.25 (m, 1H) 2.31-2.38 (m, 1H) 3.35-3.49 (m, 2H) 3.56-3.65 (m, 1H) 3.81 (dd, 1H) 4.04-4.16 (m, 4H) 4.84 (quin, 1H) 6.50 (dd, 1H) 6.70 (s, 1H) 7.05 (s, 2H) 7.17-7.22 (m, 1H) 7.27-7.35 (m, 4H) 8.12 (t, 1H) 8.60 (t, 1H)

LC-MS (method 1): Rₜ=0.98 min; MS (ESIpos): m/z=444 [M+H]⁺

### Example 208

### 2′-(6-amino-5-cyanopyridin-3-yl)-N-[(1R)-1-phenylethyl]-5′,6′-dihydrospiro[pyrrolidine-3,4′-pyrrolo[1,2-b]pyrazole]-1-carboxamide (Mixture of Stereoisomers)

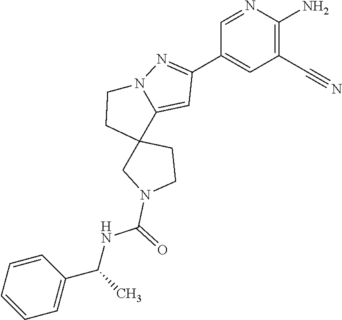 [SMILES: C[C@@H](NC(=O)N1CCC2(CCn3nc(-c4cnc(N)c(C#N)c4)cc32)C1)c1ccccc1]

Example 208 was prepared in analogy to Example 31 using (rac)-2-amino-5-[5′,6′-dihydrospiro[pyrrolidine-3,4′-pyrrolo[1,2-b]pyrazol]-2′-yl]pyridine-3-carbonitrile hydrochloride (77.0 mg, 243 μmol) and (1R)-1-phenylethan-1-amine (35.3 mg, 292 μmol).

¹H NMR (400 MHz, DMSO-d₆) δ ppm 1.37 (dd, 3H) 2.01-2.10 (m, 2H) 2.52-2.58 (m, 2H) 3.41-3.49 (m, 3H) 3.50-3.59 (m, 1H) 4.18 (t, 2H) 4.80-4.88 (m, 1H) 6.40-6.44 (m, 1H) 6.47 (dd, 1H) 6.99 (s, 2H) 7.15-7.22 (m, 1H) 7.24-7.37 (m, 4H) 8.13 (dd, 1H) 8.61 (t, 1H)

LC-MS (method 1): Rₜ=0.97 min; MS (ESIpos): m/z=429 [M+H]⁺

### Example 209

### (rac)-2-(6-amino-5-cyanopyridin-3-yl)-N-[1-(2-chlorophenyl)cyclobutyl]-6,7-dihydrospiro[pyrazolo[5,1-c][1,4]oxazine-4,3′-pyrrolidine]-1′-carboxamide

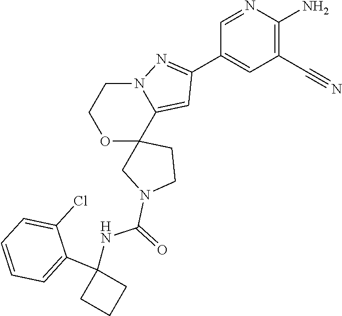 [SMILES: N#Cc1cc(-c2cc3n(n2)CCOC32CCN(C(=O)NC3(c4ccccc4Cl)CCC3)C2)cnc1N]

Example 209 was prepared in analogy to Example 31 using (rac)-2-amino-5-[6,7-dihydrospiro[pyrazolo[5,1-c][1,4]oxazine-4,3′-pyrrolidin]-2-yl]pyridine-3-carbonitrile hydrochloride (71.0 mg, 213 μmol) and 1-(2-chlorophenyl)cyclobutan-1-amine hydrochloride (55.8 mg, 256 μmol).

¹H NMR (400 MHz, DMSO-d₆) δ ppm 1.64-1.74 (m, 1H) 2.01-2.22 (m, 2H) 2.26-2.35 (m, 1H) 2.52-2.57 (m, 1H) 2.60-2.68 (m, 2H) 3.36-3.44 (m, 1H) 3.53 (br t, 1H) 3.73 (br d, 1H) 4.01-4.15 (m, 4H) 6.54 (s, 1H) 6.66 (s, 1H) 7.04 (s, 2H) 7.18-7.32 (m, 3H) 7.53 (dd, 1H) 8.09 (d, 1H) 8.58 (d, 1H)

LC-MS (method 1): Rₜ=1.14 min; MS (ESIpos): m/z=505 [M+H]⁺

### Example 210

### (rac)-2-(6-amino-5-cyanopyridin-3-yl)-N-[1-(4-fluorophenyl)cyclobutyl]-6,7-dihydrospiro[pyrazolo[5,1-c][1,4]oxazine-4,3′-pyrrolidine]-1′-carboxamide

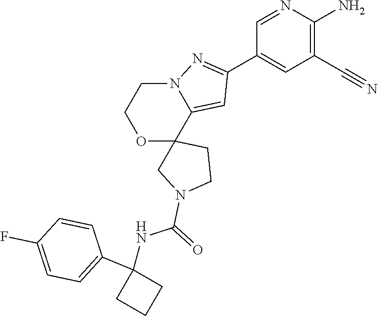 [SMILES: N#Cc1cc(-c2cc3n(n2)CCOC32CCN(C(=O)NC3(c4ccc(F)cc4)CCC3)C2)cnc1N]

Example 210 was prepared in analogy to Example 31 using (rac)-2-amino-5-[6,7-dihydrospiro[pyrazolo[5,1-c][1,4]oxazine-4,3′-pyrrolidin]-2-yl]pyridine-3-carbonitrile hydrochloride (71.0 mg, 213 μmol) and 1-(4-fluorophenyl)cyclobutan-1-amine hydrochloride (51.6 mg, 256 μmol).

¹H NMR (400 MHz, DMSO-d₆) δ ppm 1.70-1.81 (m, 1H) 1.94-2.08 (m, 1H) 2.13-2.25 (m, 1H) 2.31-2.46 (m, 5H) 3.35-3.47 (m, 2H) 3.57 (br t, 1H) 3.76 (br d, 1H) 4.04-4.16 (m, 4H) 6.67-6.75 (m, 2H) 7.01-7.14 (m, 4H) 7.41-7.47 (m, 2H) 8.11 (d, 1H) 8.59 (d, 1H)

LC-MS (method 1): Rₜ=1.07 min; MS (ESIpos): m/z=489 [M+H]⁺

### Example 211

### (rac)-2-(6-amino-5-cyanopyridin-3-yl)-N-[2-(4-fluorophenyl)propan-2-yl]-6,7-dihydrospiro[pyrazolo[5,1-c][1,4]oxazine-4,3′-pyrrolidine]-1′-carboxamide

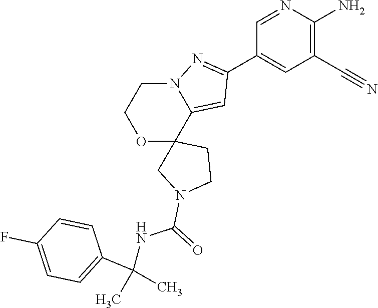 [SMILES: CC(C)(NC(=O)N1CCC2(C1)OCCn1nc(-c3cnc(N)c(C#N)c3)cc12)c1ccc(F)cc1]

Example 211 was prepared in analogy to Example 31 using (rac)-2-amino-5-[6,7-dihydrospiro[pyrazolo[5,1-c][1,4]oxazine-4,3′-pyrrolidin]-2-yl]pyridine-3-carbonitrile hydrochloride (71.0 mg, 213 μmol) and 2-(4-fluorophenyl)propan-2-amine hydrochloride (48.6 mg, 256 μmol).

¹H NMR (400 MHz, DMSO-d₆) δ ppm 1.55 (d, 6H) 2.15-2.25 (m, 1H) 2.30-2.37 (m, 1H) 3.34-3.50 (m, 2H) 3.60 (br t, 1H) 3.79 (br d, 1H) 4.05-4.17 (m, 4H) 6.10 (s, 1H) 6.71 (s, 1H) 7.01-7.11 (m, 4H) 7.35-7.37 (m, 1H) 7.38 (s, 1H) 8.12 (d, 1H) 8.60 (d, 1H)

LC-MS (method 1): Rₜ=1.05 min; MS (ESIpos): m/z=477 [M+H]⁺

### Example 212

### (rac)-2-(6-amino-5-cyanopyridin-3-yl)-N-[2-(pyridin-2-yl)propan-2-yl]-6,7-dihydrospiro[pyrazolo[5,1-c][1,4]oxazine-4,3′-pyrrolidine]-1′-carboxamide

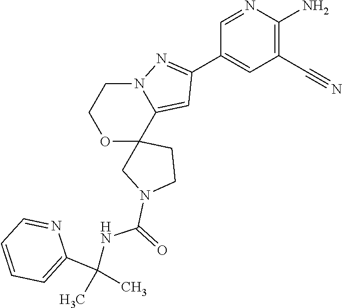 [SMILES: CC(C)(NC(=O)N1CCC2(C1)OCCn1nc(-c3cnc(N)c(C#N)c3)cc12)c1ccccn1]

Example 212 was prepared in analogy to Example 31 using (rac)-2-amino-5-[6,7-dihydrospiro[pyrazolo[5,1-c][1,4]oxazine-4,3′-pyrrolidin]-2-yl]pyridine-3-carbonitrile hydrochloride (71.0 mg, 213 μmol) and 2-(pyridin-2-yl)propan-2-amine (34.9 mg, 256 μmol).

¹H NMR (400 MHz, DMSO-d₆) δ ppm 1.58 (d, 6H) 2.18-2.27 (m, 1H) 2.52-2.54 (m, 1H) 3.36-3.52 (m, 2H) 3.63 (br t, 1H) 3.81 (d, 1H) 4.08-4.18 (m, 4H) 6.43 (s, 1H) 6.73 (s, 1H) 7.05 (s, 2H) 7.14-7.21 (m, 1H) 7.44 (d, 1H) 7.71 (t, 1H) 8.13 (d, 1H) 8.46 (d, 1H) 8.61 (d, 1H)

LC-MS (method 1): Rₜ=0.90 min; MS (ESIpos): m/z=459 [M+H]⁺

### Example 213

### (rac)-2′-(6-amino-5-cyanopyridin-3-yl)-N-[2-(pyridin-2-yl)propan-2-yl]-5′,6′-dihydrospiro[pyrrolidine-3,4′-pyrrolo[1,2-b]pyrazole]-1-carboxamide

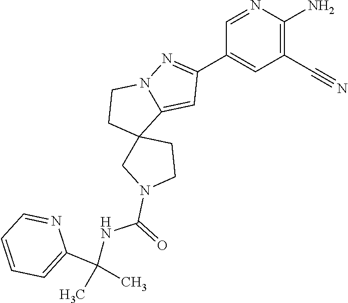 [SMILES: CC(C)(NC(=O)N1CCC2(CCn3nc(-c4cnc(N)c(C#N)c4)cc32)C1)c1ccccn1]

Example 213 was prepared in analogy to Example 31 using (rac)-2-amino-5-[5′,6′-dihydrospiro[pyrrolidine-3,4′-pyrrolo[1,2-b]pyrazol]-2′-yl]pyridine-3-carbonitrile hydrochloride (77.0 mg, 243 μmol) and 2-(pyridin-2-yl)propan-2-amine (39.7 mg, 292 μmol).

¹H NMR (400 MHz, DMSO-d₆) δ ppm 1.59 (s, 6H) 2.04-2.14 (m, 2H) 2.56 (t, 2H) 3.42-3.50 (m, 3H) 3.52-3.62 (m, 1H) 4.20 (t, 2H) 6.43 (s, 1H) 6.47 (s, 1H) 7.00 (s, 2H) 7.19 (ddd, 1H) 7.47 (d, 1H) 7.71 (td, 1H) 8.15 (d, 1H) 8.46 (d, 1H) 8.62 (d, 1H)

LC-MS (method 1): Rₜ=0.91 min; MS (ESIpos): m/z=443 [M+H]⁺

### Example 214

### 2-(6-amino-5-cyanopyridin-3-yl)-N-[1-(pyridin-4-yl)cyclobutyl]-6,7-dihydrospiro[pyrazolo[5,1-c][1,4]oxazine-4,3′-pyrrolidine]-1′-carboxamide (Enantiomer 2)

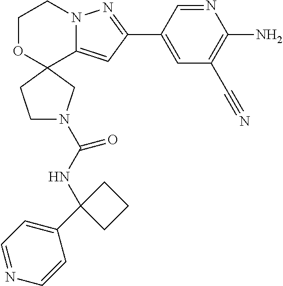 [SMILES: N#Cc1cc(-c2cc3n(n2)CCOC32CCN(C(=O)NC3(c4ccncc4)CCC3)C2)cnc1N]

Example 214 was prepared in analogy to Example 31 using 2-amino-5-[6,7-dihydrospiro[pyrazolo[5,1-c][1,4]oxazine-4,3′-pyrrolidin]-2-yl]pyridine-3-carbonitrile hydrochloride (75.0 mg, 225 μmol) and 1-(pyridin-4-yl)cyclobutan-1-amine (40.1 mg, 270 μmol).

¹H NMR (400 MHz, DMSO-d₆) δ ppm 1.77-1.88 (m, 1H) 1.96-2.08 (m, 1H) 2.15-2.28 (m, 1H) 2.32-2.47 (m, 5H) 3.37-3.51 (m, 2H) 3.60 (br t, 1H) 3.78 (br d, 1H) 4.04-4.20 (m, 4H) 6.71 (s, 1H) 6.89 (s, 1H) 7.05 (s, 2H) 7.39 (d, 2H) 8.12 (d, 1H) 8.48 (d, 2H) 8.60 (d, 1H)

[α]²⁰_{D}: +69.4° (c=1.00, DMSO)

LC-MS (method 1): Rₜ=0.84 min; MS (ESIneg): m/z=469 [M−H]⁻

### Example 215

### 2-(6-amino-5-cyanopyridin-3-yl)-N-[1-(2-chlorophenyl)cyclobutyl]-6,7-dihydrospiro[pyrazolo[5,1-c][1,4]oxazine-4,3′-pyrrolidine]-1′-carboxamide (Enantiomer 2)

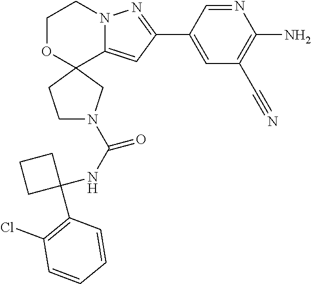 [SMILES: N#Cc1cc(-c2cc3n(n2)CCOC32CCN(C(=O)NC3(c4ccccc4Cl)CCC3)C2)cnc1N]

Example 215 was prepared in analogy to Example 31 using 2-amino-5-[6,7-dihydrospiro[pyrazolo[5,1-c][1,4]oxazine-4,3′-pyrrolidin]-2-yl]pyridine-3-carbonitrile hydrochloride (75.0 mg, 225 μmol) and 1-(2-chlorophenyl)cyclobutan-1-amine hydrochloride (54.1 mg, 248 μmol).

¹H NMR (400 MHz, DMSO-d₆) δ ppm 1.19-1.28 (m, 1H) 1.64-1.74 (m, 1H) 2.01-2.13 (m, 1H) 2.13-2.23 (m, 1H) 2.26-2.36 (m, 1H) 2.52-2.60 (m, 2H) 2.60-2.68 (m, 2H) 3.36-3.45 (m, 1H) 3.47-3.63 (m, 1H) 3.73 (br d, 1H) 4.01-4.15 (m, 4H) 6.54 (s, 1H) 6.66 (s, 1H) 7.04 (s, 2H) 7.17-7.33 (m, 3H) 7.53 (dd, 1H) 8.09 (d, 1H) 8.58 (d, 1H)

[α]²⁰_{D}: +43.5° (c=1.00, DMSO)

LC-MS (method 1): Rₜ=1.13 min; MS (ESIpos): m/z=504 [M+H]⁺

### Example 216

### 2-(6-amino-5-cyanopyridin-3-yl)-N-[2-(pyridin-2-yl)propan-2-yl]-6,7-dihydrospiro[pyrazolo[5,1-c][1,4]oxazine-4,3′-pyrrolidine]-1′-carboxamide (Enantiomer 1)

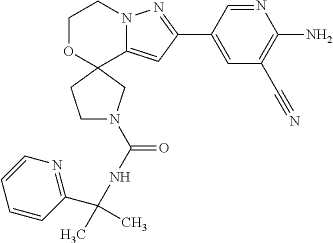 [SMILES: CC(C)(NC(=O)N1CCC2(C1)OCCn1nc(-c3cnc(N)c(C#N)c3)cc12)c1ccccn1]

Example 216 was prepared in analogy to Example 31 using 2-amino-5-[6,7-dihydrospiro[pyrazolo[5,1-c][1,4]oxazine-4,3′-pyrrolidin]-2-yl]pyridine-3-carbonitrile hydrochloride (75.0 mg, 225 μmol) and 2-(pyridin-2-yl)propan-2-amine (36.8 mg, 270 μmol).

¹H NMR (400 MHz, DMSO-d₆) δ ppm 1.05 (t, 1H) 1.58 (d, 5H) 2.15-2.30 (m, 1H) 2.31-2.41 (m, 1H) 3.42-3.54 (m, 2H) 3.62 (br t, 1H) 3.81 (br d, 1H) 4.05-4.18 (m, 4H) 6.44 (s, 1H) 6.72 (s, 1H) 7.04 (s, 2H) 7.19 (ddd, 1H) 7.45 (d, 1H) 7.72 (td, 1H) 8.13 (d, 1H) 8.46 (d, 1H) 8.61 (d, 1H)

[α]²⁰_{D}: −60.4° (c=1.00, DMSO)

LC-MS (method 1): Rₜ=0.88 min; MS (ESIneg): m/z=457 [M−H]⁻

### Example 217

### 2-(6-amino-5-cyanopyridin-3-yl)-N-[1-(2-chlorophenyl)cyclobutyl]-6,7-dihydrospiro[pyrazolo[5,1-c][1,4]oxazine-4,3′-pyrrolidine]-1′-carboxamide (Enantiomer 1)

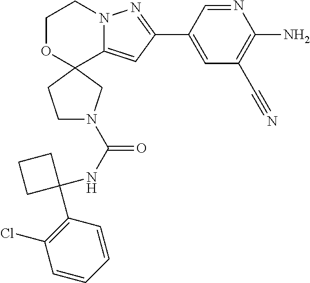 [SMILES: N#Cc1cc(-c2cc3n(n2)CCOC32CCN(C(=O)NC3(c4ccccc4Cl)CCC3)C2)cnc1N]

Example 217 was prepared in analogy to Example 31 using 2-amino-5-[6,7-dihydrospiro[pyrazolo[5,1-c][1,4]oxazine-4,3′-pyrrolidin]-2-yl]pyridine-3-carbonitrile hydrochloride (75.0 mg, 225 μmol) and 1-(2-chlorophenyl)cyclobutan-1-amine hydrochloride (54.1 mg, 248 μmol).

¹H NMR (400 MHz, DMSO-d₆) δ ppm 1.64-1.74 (m, 1H) 2.01-2.23 (m, 2H) 2.25-2.35 (m, 1H) 2.52-2.60 (m, 1H) 2.61-2.68 (m, 2H) 3.36-3.45 (m, 1H) 3.53 (br t, 1H) 3.74 (br d, 1H) 4.01-4.17 (m, 4H) 6.54 (s, 1H) 6.66 (s, 1H) 7.04 (s, 2H) 7.17-7.34 (m, 3H) 7.53 (dd, 1H) 8.09 (d, 1H) 8.58 (d, 1H)

[α]²⁰_{D}: −69.8° (c=1.00, DMSO)

LC-MS (method 1): Rₜ=1.13 min; MS (ESIpos): m/z=504 [M+H]⁺

### Example 218

### 2′-(6-amino-5-cyanopyridin-3-yl)-N-[1-(pyridin-3-yl)cyclobutyl]-5′,6′-dihydrospiro[azetidine-3,4′-pyrrolo[1,2-b]pyrazole]-1-carboxamide

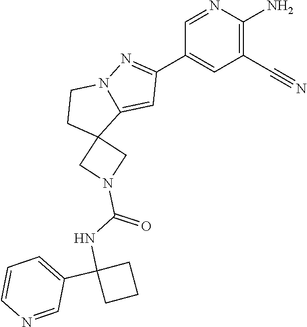 [SMILES: N#Cc1cc(-c2cc3n(n2)CCC32CN(C(=O)NC3(c4cccnc4)CCC3)C2)cnc1N]

Example 218 was prepared in analogy to Example 31 using 2-amino-5-(5′,6′-dihydrospiro[azetidine-3,4′-pyrrolo[1,2-b]pyrazol]-2′-yl)pyridine-3-carbonitrile hydrochloride (75.0 mg, 248 μmol) and 1-(pyridin-3-yl)cyclobutan-1-amine (44.1 mg, 297 μmol).

¹H NMR (500 MHz, DMSO-d₆) δ ppm 1.05 (t, 1H) 1.76-1.86 (m, 1H) 1.96-2.11 (m, 1H) 2.41-2.48 (m, 3H) 2.83 (t, 2H) 3.95-4.05 (m, 4H) 4.12 (t, 2H) 6.67 (s, 1H) 7.00 (s, 2H) 7.21 (s, 1H) 7.33-7.36 (m, 1H) 7.79 (dt, 1H) 8.16 (d, 1H) 8.41 (dd, 1H) 8.64 (d, 2H)

LC-MS (method 1): Rₜ=0.84 min; MS (ESIneg): m/z=439 [M−H]⁻

### Example 219

### 2′-(6-amino-5-cyanopyridin-3-yl)-N-[1-(pyridin-2-yl)cyclobutyl]-5′,6′-dihydrospiro[azetidine-3,4′-pyrrolo[1,2-b]pyrazole]-1-carboxamide

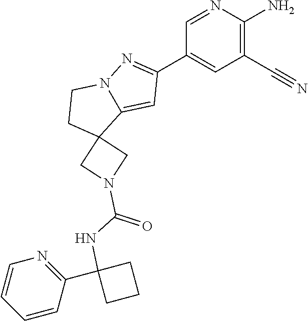 [SMILES: N#Cc1cc(-c2cc3n(n2)CCC32CN(C(=O)NC3(c4ccccn4)CCC3)C2)cnc1N]

Example 219 was prepared in analogy to Example 31 using 2-amino-5-(5′,6′-dihydrospiro[azetidine-3,4′-pyrrolo[1,2-b]pyrazol]-2′-yl)pyridine-3-carbonitrile hydrochloride (75.0 mg, 248 μmol) and 1-(pyridin-2-yl)cyclobutan-1-amine (44.1 mg, 297 μmol).

¹H NMR (500 MHz, DMSO-d₆) δ ppm 1.83-2.06 (m, 2H) 2.34-2.41 (m, 2H) 2.83-2.90 (m, 2H) 3.99-4.10 (m, 5H) 4.14 (t, 3H) 6.71 (s, 1H) 7.00 (s, 2H) 7.17-7.23 (m, 2H) 7.38 (d, 1H) 7.73 (td, 1H) 8.18 (d, 1H) 8.55 (d, 1H) 8.65 (d, 1H)

LC-MS (method 1): Rₜ=0.85 min; MS (ESIpos): m/z=441 [M+H]⁺

### Example 220

### 2-(6-amino-5-cyanopyridin-3-yl)-N-[1-(pyridin-4-yl)cyclobutyl]-6,7-dihydrospiro[pyrazolo[5,1-c][1,4]oxazine-4,3′-pyrrolidine]-1′-carboxamide (Enantiomer 1)

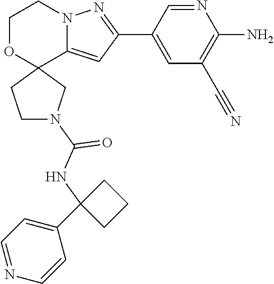 [SMILES: N#Cc1cc(-c2cc3n(n2)CCOC32CCN(C(=O)NC3(c4ccncc4)CCC3)C2)cnc1N]

Example 220 was prepared in analogy to Example 31 using 2-amino-5-[6,7-dihydrospiro[pyrazolo[5,1-c][1,4]oxazine-4,3′-pyrrolidin]-2-yl]pyridine-3-carbonitrile hydrochloride (75.0 mg, 225 μmol) and 1-(pyridin-4-yl)cyclobutan-1-amine (40.1 mg, 270 μmol).

¹H NMR (400 MHz, DMSO-d₆) δ ppm 1.77-1.91 (m, 1H) 1.97-2.10 (m, 1H) 2.16-2.29 (m, 1H) 2.30-2.47 (m, 5H) 3.36-3.46 (m, 2H) 3.58-3.64 (m, 1H) 3.76-3.81 (m, 1H) 4.04-4.20 (m, 4H) 6.69-6.74 (m, 1H) 6.96 (s, 1H) 7.01-7.09 (m, 2H) 7.49-7.54 (m, 2H) 8.12 (d, 1H) 8.52-8.57 (m, 2H) 8.60 (d, 1H)

[α]²⁰_{D}: −155.6° (c=1.00, DMSO)

LC-MS (method 1): Rₜ=0.83 min; MS (ESIneg): m/z=469 [M−H]⁻

### Example 221

### 2′-(6-amino-5-cyanopyridin-3-yl)-N-(1-phenylcyclobutyl)-5′,6′-dihydrospiro[azetidine-3,4′-pyrrolo[1,2-b]pyrazole]-1-carboxamide

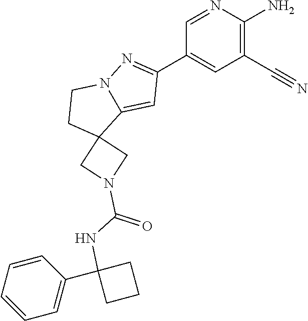 [SMILES: N#Cc1cc(-c2cc3n(n2)CCC32CN(C(=O)NC3(c4ccccc4)CCC3)C2)cnc1N]

Example 221 was prepared in analogy to Example 31 using 2-amino-5-(5′,6′-dihydrospiro[azetidine-3,4′-pyrrolo[1,2-b]pyrazol]-2′-yl)pyridine-3-carbonitrile hydrochloride (75.0 mg, 248 μmol) and 1-phenylcyclobutan-1-amine hydrochloride (54.6 mg, 297 μmol).

¹H NMR (400 MHz, DMSO-d₆) δ ppm 1.73-1.84 (m, 1H) 1.95-2.10 (m, 1H) 2.34-2.48 (m, 4H) 2.83 (t, 2H) 3.93-4.04 (m, 4H) 4.12 (t, 2H) 6.66 (s, 1H) 7.00 (s, 2H) 7.08 (s, 1H) 7.14-7.22 (m, 1H) 7.32 (t, 2H) 7.40-7.45 (m, 2H) 8.16 (d, 1H) 8.64 (d, 1H)

LC-MS (method 1): Rₜ=1.01 min; MS (ESIneg): m/z=438 [M−H]⁻

### Example 222

### 2′-(6-amino-5-cyanopyridin-3-yl)-N-[(3S)-2,3-dihydro-1-benzofuran-3-yl]-5′,6′-dihydrospiro[pyrrolidine-3,4′-pyrrolo[1,2-b]pyrazole]-1-carboxamide (Mixture of Stereoisomers)

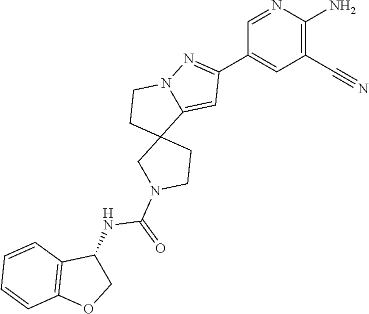 [SMILES: N#Cc1cc(-c2cc3n(n2)CCC32CCN(C(=O)N[C@@H]3COc4ccccc43)C2)cnc1N]

Example 222 was prepared in analogy to Example 31 using (rac)-2-amino-5-[5′,6′-dihydrospiro[pyrrolidine-3,4′-pyrrolo[1,2-b]pyrazol]-2′-yl]pyridine-3-carbonitrile hydrochloride (100 mg, 316 μmol) and (3S)-2,3-dihydro-1-benzofuran-3-amine (51.2 mg, 379 μmol).

¹H NMR (400 MHz, DMSO-d₆) δ ppm 2.05 (t, 2H) 2.52-2.56 (m, 2H) 3.35-3.57 (m, 4H) 4.17 (t, 2H) 4.24 (dt, 1H) 4.66 (td, 1H) 5.43-5.50 (m, 1H) 6.44 (d, 1H) 6.73-6.90 (m, 3H) 6.99 (s, 2H) 7.18 (tdd, 1H) 7.33 (dd, 1H) 8.14 (dd, 1H) 8.61 (d, 1H)

LC-MS (method 1): Rₜ=0.92 min; MS (ESIneg): m/z=440 [M−H]⁻

### Example 223

### 2-(6-amino-5-cyanopyridin-3-yl)-N-[(3S)-2,3-dihydro-1-benzofuran-3-yl]-6,7-dihydrospiro[pyrazolo[5,1-c][1,4]oxazine-4,3′-pyrrolidine]-1′-carboxamide (Mixture of Stereoisomers)

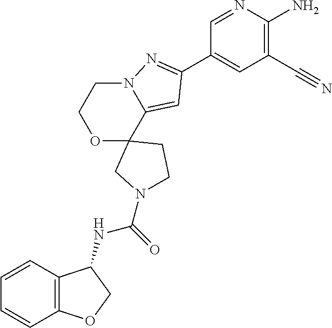 [SMILES: N#Cc1cc(-c2cc3n(n2)CCOC32CCN(C(=O)N[C@@H]3COc4ccccc43)C2)cnc1N]

Example 223 was prepared in analogy to Example 31 using (rac)-2-amino-5-[6,7-dihydrospiro[pyrazolo[5,1-c][1,4]oxazine-4,3′-pyrrolidin]-2-yl]pyridine-3-carbonitrile hydrochloride (95.0 mg, 285 μmol) and (3S)-2,3-dihydro-1-benzofuran-3-amine (46.3 mg, 343 μmol).

¹H NMR (400 MHz, DMSO-d₆) δ ppm 2.14-2.26 (m, 1H) 2.29-2.40 (m, 1H) 3.35-3.49 (m, 2H) 3.60 (dt, 1H) 3.82 (br t, 1H) 4.03-4.16 (m, 4H) 4.23 (ddd, 1H) 4.66 (t, 1H) 5.42-5.49 (m, 1H) 6.69 (d, 1H) 6.81 (d, 2H) 6.88 (tdd, 1H) 7.05 (s, 2H) 7.19 (t, 1H) 7.32 (d, 1H) 8.12 (dd, 1H) 8.60 (dd, 1H)

LC-MS (method 1): Rₜ=0.92 min; MS (ESIpos): m/z=458 [M+H]⁺

### Example 224

### 2-(6-amino-5-cyanopyridin-3-yl)-N-[(1R)-1-(2-fluorophenyl)ethyl]-6,7-dihydrospiro[pyrazolo[5,1-c][1,4]oxazine-4,3′-pyrrolidine]-1′-carboxamide (Mixture of Stereoisomers)

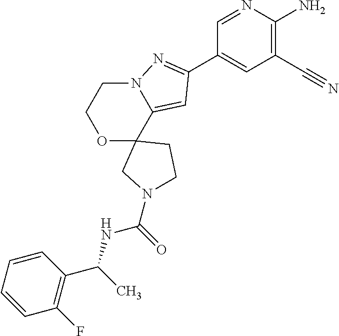 [SMILES: C[C@@H](NC(=O)N1CCC2(C1)OCCn1nc(-c3cnc(N)c(C#N)c3)cc12)c1ccccc1F]

Example 224 was prepared in analogy to Example 31 using (rac)-2-amino-5-[6,7-dihydrospiro[pyrazolo[5,1-c][1,4]oxazine-4,3′-pyrrolidin]-2-yl]pyridine-3-carbonitrile hydrochloride (95.0 mg, 285 μmol) and (1R)-1-(2-fluorophenyl)ethan-1-amine (47.7 mg, 343 μmol).

¹H NMR (400 MHz, DMSO-d₆) δ ppm 1.35 (d, 3H) 2.16-2.27 (m, 1H) 2.31-2.40 (m, 1H) 3.36-3.51 (m, 2H) 3.57-3.67 (m, 1H) 3.82 (t, 1H) 4.04-4.17 (m, 4H) 5.07-5.14 (m, 1H) 6.59 (dd, 1H) 6.70 (s, 1H) 7.05 (s, 2H) 7.09-7.20 (m, 2H) 7.21-7.29 (m, 1H) 7.42-7.48 (m, 1H) 8.12 (dd, 1H) 8.59-8.62 (m, 1H)

LC-MS (method 1): Rₜ=0.98 min; MS (ESIneg): m/z=460 [M−H]⁻

### Example 225

### 2′-(6-amino-5-cyanopyridin-3-yl)-N-[(1R)-1-(2-fluorophenyl)ethyl]-5′,6′-dihydrospiro[pyrrolidine-3,4′-pyrrolo[1,2-b]pyrazole]-1-carboxamide (Mixture of Stereoisomers)

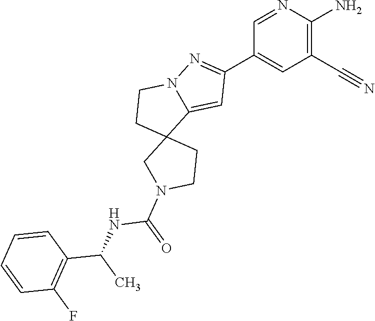 [SMILES: C[C@@H](NC(=O)N1CCC2(CCn3nc(-c4cnc(N)c(C#N)c4)cc32)C1)c1ccccc1F]

Example 225 was prepared in analogy to Example 31 using (rac)-2-amino-5-[5′,6′-dihydrospiro[pyrrolidine-3,4′-pyrrolo[1,2-b]pyrazol]-2′-yl]pyridine-3-carbonitrile hydrochloride (100 mg, 316 μmol) and (1R)-1-(2-fluorophenyl)ethan-1-amine (52.7 mg, 379 μmol).

¹H NMR (400 MHz, DMSO-d₆) δ ppm 1.35 (dd, 3H) 2.07 (br t, 2H) 2.52-2.57 (m, 2H) 3.42-3.61 (m, 4H) 4.19 (t, 2H) 5.07-5.15 (m, 1H) 6.40-6.45 (m, 1H) 6.56 (dd, 1H) 6.99 (s, 2H) 7.07-7.20 (m, 2H) 7.20-7.30 (m, 1H) 7.40-7.51 (m, 1H) 8.13 (dd, 1H) 8.61 (t, 1H)

LC-MS (method 1): Rₜ=0.98 min; MS (ESIneg): m/z=444 [M−H]⁻

### Example 226

### (rac)-2′-(6-amino-5-cyanopyridin-3-yl)-N-[2-(4-fluorophenyl)propan-2-yl]-5′,6′-dihydrospiro[pyrrolidine-3,4′-pyrrolo[1,2-b]pyrazole]-1-carboxamide

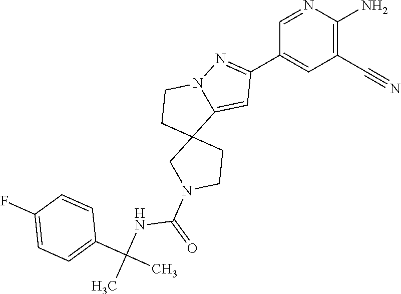 [SMILES: CC(C)(NC(=O)N1CCC2(CCn3nc(-c4cnc(N)c(C#N)c4)cc32)C1)c1ccc(F)cc1]

Example 226 was prepared in analogy to Example 31 using (rac)-2-amino-5-[5′,6′-dihydrospiro[pyrrolidine-3,4′-pyrrolo[1,2-b]pyrazol]-2′-yl]pyridine-3-carbonitrile hydrochloride (100 mg, 316 μmol) and 2-(4-fluorophenyl)propan-2-amine (58.0 mg, 379 μmol).

¹H NMR (400 MHz, DMSO-d₆) δ ppm 1.52-1.58 (m, 6H) 2.00-2.11 (m, 2H) 2.52-2.58 (m, 2H) 3.35-3.47 (m, 3H) 3.48-3.60 (m, 1H) 4.19 (t, 2H) 6.07 (s, 1H) 6.44 (s, 1H) 6.98-7.02 (m, 2H) 7.05 (t, 2H) 7.35-7.37 (m, 1H) 7.38 (s, 1H) 8.14 (d, 1H) 8.62 (d, 1H)

LC-MS (method 1): Rₜ=1.04 min; MS (ESIneg): m/z=458 [M−H]⁻

### Example 227

### (rac)-2-(6-amino-5-cyanopyridin-3-yl)-N-[2-(1-methyl-1H-1,2,3-triazol-4-yl)propan-2-yl]-6,7-dihydrospiro[pyrazolo[5,1-c][1,4]oxazine-4,3′-pyrrolidine]-1′-carboxamide

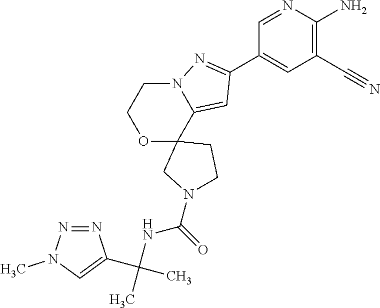 [SMILES: Cn1cc(C(C)(C)NC(=O)N2CCC3(C2)OCCn2nc(-c4cnc(N)c(C#N)c4)cc23)nn1]

Example 227 was prepared in analogy to Example 31 using (rac)-2-amino-5-[6,7-dihydrospiro[pyrazolo[5,1-c][1,4]oxazine-4,3′-pyrrolidin]-2-yl]pyridine-3-carbonitrile hydrochloride (95.0 mg, 285 μmol) and 2-(1-methyl-1H-1,2,3-triazol-4-yl)propan-2-amine hydrochloride (73.0 mg, 343 μmol).

¹H NMR (400 MHz, DMSO-d₆) δ ppm 1.60 (d, 6H) 2.13-2.24 (m, 1H) 2.28-2.36 (m, 1H) 3.35-3.47 (m, 2H) 3.58 (br t, 1H) 3.72-3.82 (m, 1H) 3.98 (s, 3H) 4.04-4.17 (m, 4H) 5.96 (s, 1H) 6.70 (s, 1H) 7.04 (s, 2H) 7.79 (s, 1H) 8.13 (d, 1H) 8.61 (d, 1H)

LC-MS (method 1): Rₜ=0.75 min; MS (ESIpos): m/z=464 [M+H]⁺

### Example 228

### (rac)-2-(6-amino-5-cyanopyridin-3-yl)-N-[(1R)-1-(5-fluoropyridin-3-yl)ethyl]-6,7-dihydrospiro[pyrazolo[5,1-c][1,4]oxazine-4,3′-pyrrolidine]-1′-carboxamide

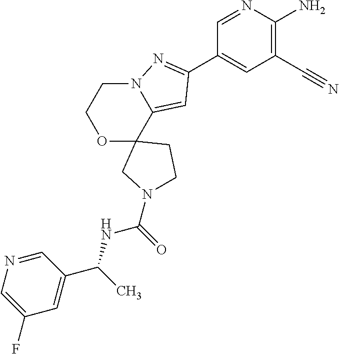 [SMILES: C[C@@H](NC(=O)N1CCC2(C1)OCCn1nc(-c3cnc(N)c(C#N)c3)cc12)c1cncc(F)c1]

Example 228 was prepared in analogy to Example 31 using (rac)-2-amino-5-[6,7-dihydrospiro[pyrazolo[5,1-c][1,4]oxazine-4,3′-pyrrolidin]-2-yl]pyridine-3-carbonitrile hydrochloride (93.0 mg, 279 μmol) and (1R)-1-(5-fluoropyridin-3-yl)ethan-1-amine (47.0 mg, 335 μmol).

¹H NMR (400 MHz, DMSO-d₆) δ ppm 1.41 (d, 3H) 2.16-2.27 (m, 1H) 2.31-2.40 (m, 1H) 3.35-3.52 (m, 2H) 3.55-3.67 (m, 1H) 3.74-3.87 (m, 1H) 4.05-4.17 (m, 4H) 4.87-4.96 (m, 1H) 6.63 (dd, 1H) 6.70 (d, 1H) 7.05 (s, 2H) 7.68 (ddt, 1H) 8.12 (t, 1H) 8.41-8.46 (m, 2H) 8.60 (t, 1H)

LC-MS (method 1): Rₜ=0.82 min; MS (ESIpos): m/z=463 [M+H]⁺

### Example 229

### (rac)-2′-(6-amino-5-cyanopyridin-3-yl)-N-(1-phenylcyclobutyl)-5′,6′-dihydrospiro[pyrrolidine-3,4′-pyrrolo[1,2-b]pyrazole]-1-carboxamide

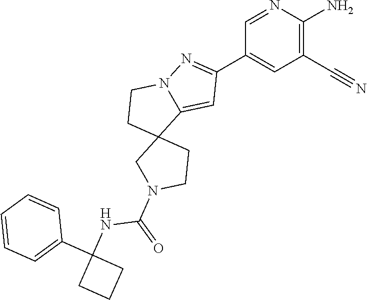 [SMILES: N#Cc1cc(-c2cc3n(n2)CCC32CCN(C(=O)NC3(c4ccccc4)CCC3)C2)cnc1N]

Example 229 was prepared in analogy to Example 31 using (rac)-2-amino-5-[5′,6′-dihydrospiro[pyrrolidine-3,4′-pyrrolo[1,2-b]pyrazol]-2′-yl]pyridine-3-carbonitrile hydrochloride (77.0 mg, 243 μmol) and 1-phenylcyclobutan-1-amine hydrochloride (53.6 mg, 292 μmol).

¹H NMR (400 MHz, DMSO-d₆) δ ppm 1.72-1.83 (m, 1H) 1.95-2.05 (m, 1H) 2.14-2.25 (m, 1H) 2.31-2.47 (m, 5H) 3.35-3.47 (m, 2H) 3.58 (br t, 1H) 3.77 (br d, 1H) 4.04-4.16 (m, 4H) 6.67-6.73 (m, 2H) 7.05 (s, 2H) 7.13-7.20 (m, 1H) 7.29 (t, 2H) 7.42 (d, 2H) 8.11 (d, 1H) 8.59 (d, 1H)

LC-MS (method 1): Rₜ=1.06 min; MS (ESIpos): m/z=454 [M+H]⁺

### Example 230

### (rac)-2′-(6-amino-5-cyanopyridin-3-yl)-N-[1-(4-fluorophenyl)cyclobutyl]-5′,6′-dihydrospiro[pyrrolidine-3,4′-pyrrolo[1,2-b]pyrazole]-1-carboxamide

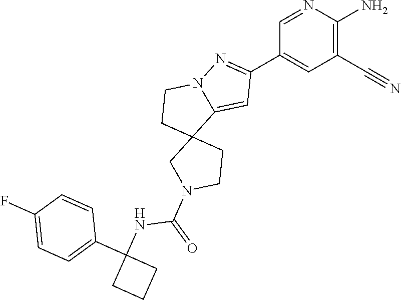 [SMILES: N#Cc1cc(-c2cc3n(n2)CCC32CCN(C(=O)NC3(c4ccc(F)cc4)CCC3)C2)cnc1N]

Example 230 was prepared in analogy to Example 31 using (rac)-2-amino-5-[5′,6′-dihydrospiro[pyrrolidine-3,4′-pyrrolo[1,2-b]pyrazol]-2′-yl]pyridine-3-carbonitrile hydrochloride (77.0 mg, 243 μmol) and 1-(4-fluorophenyl)cyclobutan-1-amine hydrochloride (58.8 mg, 292 μmol).

¹H NMR (400 MHz, DMSO-d₆) δ ppm 1.70-1.81 (m, 1H) 1.93-2.10 (m, 3H) 2.34-2.47 (m, 4H) 2.52-2.57 (m, 2H) 3.35-3.44 (m, 3H) 3.46-3.56 (m, 1H) 4.18 (t, 2H) 6.42 (s, 1H) 6.71 (s, 1H) 6.99 (s, 2H) 7.05-7.12 (m, 2H) 7.41-7.47 (m, 2H) 8.13 (d, 1H) 8.60 (d, 1H)

LC-MS (method 1): Rₜ=1.07 min; MS (ESIpos): m/z=473 [M+H]⁺

### Example 231

### (rac)-2-(6-amino-5-cyanopyridin-3-yl)-N-[1-(pyridin-4-yl)cyclobutyl]-6,7-dihydrospiro[pyrazolo[5,1-c][1,4]oxazine-4,3′-pyrrolidine]-1′-carboxamide

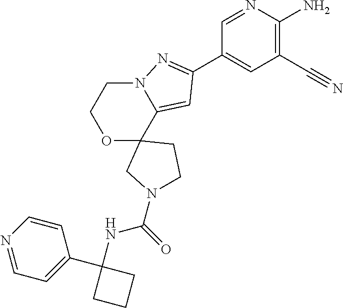 [SMILES: N#Cc1cc(-c2cc3n(n2)CCOC32CCN(C(=O)NC3(c4ccncc4)CCC3)C2)cnc1N]

Example 231 was prepared in analogy to Example 31 using (rac)-2-amino-5-[6,7-dihydrospiro[pyrazolo[5,1-c][1,4]oxazine-4,3′-pyrrolidin]-2-yl]pyridine-3-carbonitrile hydrochloride (71.0 mg, 213 μmol) and 1-(pyridin-4-yl)cyclobutan-1-amine (37.9 mg, 256 μmol).

¹H NMR (400 MHz, DMSO-d₆) δ ppm 1.79-1.88 (m, 1H) 1.97-2.07 (m, 1H) 2.21 (br d, 1H) 2.35-2.47 (m, 5H) 3.36-3.50 (m, 2H) 3.60 (br t, 1H) 3.78 (br d, 1H) 4.05-4.17 (m, 4H) 6.71 (s, 1H) 6.89 (s, 1H) 7.05 (s, 2H) 7.39 (d, 2H) 8.12 (d, 1H) 8.48 (d, 2H) 8.60 (d, 1H)

LC-MS (method 1): Rₜ=0.86 min; MS (ESIpos): m/z=471 [M+H]⁺

### Example 232

### (rac)-2′-(6-amino-5-cyanopyridin-3-yl)-N-[1-(pyridin-4-yl)cyclobutyl]-5′,6′-dihydrospiro[pyrrolidine-3,4′-pyrrolo[1,2-b]pyrazole]-1-carboxamide

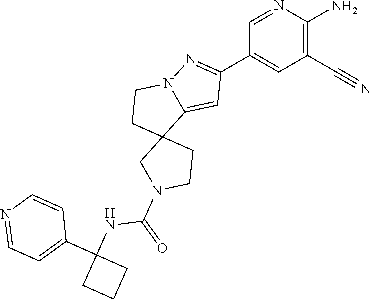 [SMILES: N#Cc1cc(-c2cc3n(n2)CCC32CCN(C(=O)NC3(c4ccncc4)CCC3)C2)cnc1N]

Example 232 was prepared in analogy to Example 31 using (rac)-2-amino-5-[5′,6′-dihydrospiro[pyrrolidine-3,4′-pyrrolo[1,2-b]pyrazol]-2′-yl]pyridine-3-carbonitrile hydrochloride (77.0 mg, 243 μmol) and 1-(pyridin-4-yl)cyclobutan-1-amine (43.2 mg, 292 μmol).

¹H NMR (400 MHz, DMSO-d₆) δ ppm 1.79-1.89 (m, 1H) 1.97-2.11 (m, 3H) 2.35-2.48 (m, 4H) 2.53-2.57 (m, 2H) 3.44 (s, 3H) 3.55 (br d, 1H) 4.19 (t, 2H) 6.44 (s, 1H) 6.88 (s, 1H) 7.00 (s, 2H) 7.42 (d, 2H) 8.14 (d, 1H) 8.48 (d, 2H) 8.61 (d, 1H)

LC-MS (method 1): Rₜ=0.86 min; MS (ESIpos): m/z=455 [M+H]⁺

### Example 233

### (rac)-2-(6-amino-5-cyanopyridin-3-yl)-N-(1-phenylcyclobutyl)-6,7-dihydrospiro[pyrazolo[5,1-c][1,4]oxazine-4,3′-pyrrolidine]-1′-carboxamide

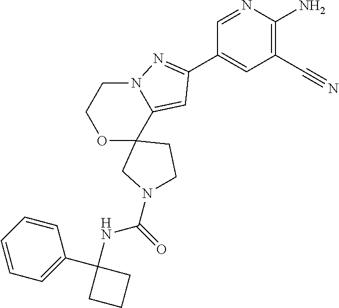 [SMILES: N#Cc1cc(-c2cc3n(n2)CCOC32CCN(C(=O)NC3(c4ccccc4)CCC3)C2)cnc1N]

Example 233 was prepared in analogy to Example 31 using (rac)-2-amino-5-[6,7-dihydrospiro[pyrazolo[5,1-c][1,4]oxazine-4,3′-pyrrolidin]-2-yl]pyridine-3-carbonitrile hydrochloride (71.0 mg, 213 μmol) and 1-phenylcyclobutan-1-amine hydrochloride (47.0 mg, 256 μmol).

¹H NMR (400 MHz, DMSO-d₆) δ ppm 1.77 (td, 1H) 1.94-2.09 (m, 3H) 2.33-2.46 (m, 3H) 2.52-2.55 (m, 3H) 3.35-3.45 (m, 3H) 3.46-3.56 (m, 1H) 4.18 (t, 2H) 6.39 (s, 1H) 6.70 (s, 1H) 7.00 (s, 2H) 7.13-7.18 (m, 1H) 7.28 (t, 2H) 7.43 (d, 2H) 8.11 (d, 1H) 8.60 (d, 1H)

LC-MS (method 1): Rₜ=1.06 min; MS (ESIpos): m/z=470 [M+H]⁺

### Example 234

### 2′-(6-amino-5-cyanopyridin-3-yl)-N-[(1R)-1-(1,3,4-trimethyl-1H-pyrazol-5-yl)ethyl]-5′,6′-dihydrospiro[azetidine-3,4′-pyrrolo[1,2-b]pyrazole]-1-carboxamide

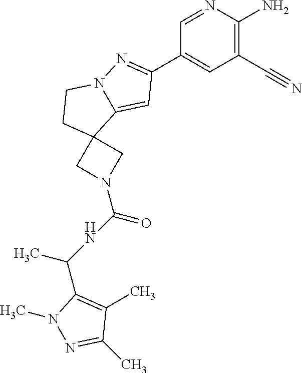 [SMILES: Cc1nn(C)c(C(C)NC(=O)N2CC3(CCn4nc(-c5cnc(N)c(C#N)c5)cc43)C2)c1C]

Example 234 was prepared in analogy to Example 31 using 2-amino-5-(5′,6′-dihydrospiro[azetidine-3,4′-pyrrolo[1,2-b]pyrazol]-2′-yl)pyridine-3-carbonitrile (75.0 mg, 282 μmol) and 1-(1,3,4-trimethyl-1H-pyrazol-5-yl)ethan-1-amine (51.8 mg, 338 μmol).

¹H NMR (400 MHz, DMSO-d₆) δ ppm 1.40 (d, 3H) 1.96 (s, 3H) 1.99 (s, 3H) 2.83 (t, 2H) 3.71 (s, 3H) 3.93-4.05 (m, 4H) 4.11 (t, 2H) 4.88 (t, 1H) 6.65 (s, 1H) 6.92 (d, 1H) 7.00 (s, 2H) 8.15 (d, 1H) 8.63 (d, 1H)

LC-MS (Method 1): Rt=0.83 min; MS (ESIpos): m/z=446 [M+H]⁺

### Example 235

### (rac)-2-(6-amino-5-cyanopyridin-3-yl)-N-[2-(pyridin-4-yl) propan-2-yl]-6,7-dihydrospiro[pyrazolo[5,1-c][1,4]oxazine-4,3′-pyrrolidine]-1′-carboxamide

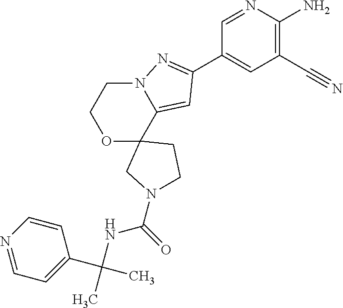 [SMILES: CC(C)(NC(=O)N1CCC2(C1)OCCn1nc(-c3cnc(N)c(C#N)c3)cc12)c1ccncc1]

Example 235 was prepared in analogy to Example 31 using (rac)-2-amino-5-[6,7-dihydrospiro[pyrazolo[5,1-c][1,4]oxazine-4,3′-pyrrolidin]-2-yl]pyridine-3-carbonitrile hydrochloride (93.0 mg, 279 μmol) and 2-(pyridin-4-yl)propan-2-amine (45.7 mg, 335 μmol).

¹H NMR (400 MHz, DMSO-d₆) δ ppm 1.54 (s, 6H) 2.15-2.27 (m, 1H) 2.29-2.40 (m, 1H) 3.35-3.51 (m, 2H) 3.61 (br t, 1H) 3.80 (br d, 1H) 4.07-4.18 (m, 4H) 6.25 (s, 1H) 6.72 (s, 1H) 7.05 (s, 2H) 7.32 (d, 2H) 8.13 (d, 1H) 8.44 (d, 2H) 8.61 (d, 1H)

LC-MS (method 1): Rₜ=0.82 min; MS (ESIneg): m/z=457 [M−H]⁻

### Example 236

### 2′-[6-amino-5-(difluoromethoxy)pyridin-3-yl]-N-[1-(pyridin-3-yl)cyclobutyl]-5′,6′-dihydrospiro[azetidine-3,4′-pyrrolo[1,2-b]pyrazole]-1-carboxamide

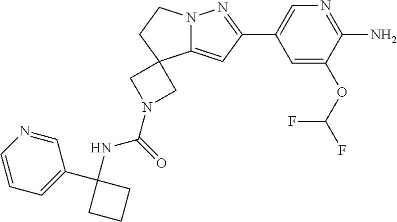 [SMILES: Nc1ncc(-c2cc3n(n2)CCC32CN(C(=O)NC3(c4cccnc4)CCC3)C2)cc1OC(F)F]

Example 236 was prepared in analogy to Example 31 using 3-(difluoromethoxy)-5-(5′,6′-dihydrospiro[azetidine-3,4′-pyrrolo[1,2-b]pyrazol]-2′-yl)pyridin-2-amine-hydrochloride salt (75.0 mg, 218 μmol) and 1-(pyridin-3-yl)cyclobutan-1-amine (35.6 mg, 240 μmol).

LC-MS (method 1): Rₜ=0.88 min; MS (ESIpos): m/z=482 [M+H]⁺

¹H-NMR (400 MHz, DMSO-d6) δ[ppm]: 1.066 (3.99), 1.235 (0.40), 1.245 (1.29), 1.254 (0.89), 1.261 (1.42), 1.267 (1.40), 1.284 (1.24), 1.779 (0.54), 1.785 (0.47), 1.796 (1.09), 1.803 (1.07), 1.813 (0.86), 1.819 (1.04), 1.824 (1.35), 1.834 (0.53), 1.841 (0.89), 2.008 (0.71), 2.024 (1.22), 2.031 (1.03), 2.036 (0.80), 2.042 (0.82), 2.047 (1.18), 2.052 (1.09), 2.063 (0.60), 2.069 (0.58), 2.073 (0.72), 2.413 (0.71), 2.430 (1.13), 2.442 (2.62), 2.458 (5.56), 2.479 (6.00), 2.518 (2.60), 2.522 (1.93), 2.727 (3.16), 2.815 (3.09), 2.833 (5.23), 2.850 (3.40), 2.887 (3.90), 3.979 (2.66), 4.000 (10.91), 4.009 (11.07), 4.029 (2.72), 4.095 (3.45), 4.113 (5.53), 4.130 (3.39), 6.207 (3.15), 6.616 (16.00), 7.005 (3.56), 7.023 (0.46), 7.150 (0.41), 7.190 (7.34), 7.219 (7.44), 7.374 (4.95), 7.386 (2.50), 7.388 (2.55), 7.396 (2.65), 7.408 (2.64), 7.652 (5.00), 7.654 (5.21), 7.656 (5.19), 7.831 (2.05), 7.836 (2.71), 7.841 (2.23), 7.851 (1.93), 7.857 (2.52), 7.861 (1.96), 7.950 (0.50), 8.236 (8.81), 8.241 (8.52), 8.423 (4.19), 8.427 (4.26), 8.435 (4.31), 8.439 (3.99), 8.656 (5.05), 8.661 (5.22), 8.663 (4.93).

### Example 237

### (rac)-2′-[6-amino-5-(trifluoromethoxy)pyridin-3-yl]-N-[2-(1-methyl-1H-1,2,3-triazol-4-yl)propan-2-yl]-5′,6′-dihydrospiro[pyrrolidine-3,4′-pyrrolo[1,2-b]pyrazole]-1-carboxamide

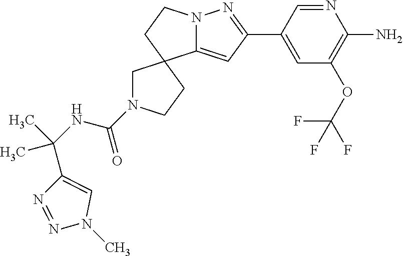 [SMILES: Cn1cc(C(C)(C)NC(=O)N2CCC3(CCn4nc(-c5cnc(N)c(OC(F)(F)F)c5)cc43)C2)nn1]

Example 237 was prepared in analogy to Example 31 using (rac)-5-[5′,6′-dihydrospiro[pyrrolidine-3,4′-pyrrolo[1,2-b]pyrazol]-2′-yl]-3-(trifluoromethoxy)pyridin-2-amine (75.0 mg, 221 μmol) and 2-(1-methyl-1H-1,2,3-triazol-4-yl)propan-2-amine-hydrochloride salt (56.5 mg, 265 μmol).

LC-MS (method 2): Rt=0.85 min; MS (ESIpos): m/z=506 [M+H]⁺

1H-NMR (400 MHz, DMSO-d6) δ[ppm]: 0.969 (2.09), 0.984 (1.89), 1.510 (0.99), 1.593 (8.69), 1.601 (8.54), 2.023 (0.61), 2.038 (1.12), 2.055 (1.35), 2.074 (0.97), 2.084 (2.27), 2.336 (0.46), 2.518 (6.88), 2.522 (5.45), 2.539 (1.81), 3.399 (1.40), 3.420 (4.08), 3.443 (0.74), 3.502 (0.51), 3.521 (0.74), 3.535 (0.56), 3.542 (0.48), 3.975 (16.00), 4.136 (0.43), 4.159 (1.48), 4.176 (2.19), 4.194 (1.38), 5.923 (2.55), 6.428 (5.76), 6.499 (3.95), 7.756 (1.45), 7.760 (2.06), 7.795 (5.86), 8.312 (0.41), 8.317 (0.43), 8.336 (2.73), 8.341 (2.73).

### Example 238

### 2′-[6-amino-5-(trifluoromethoxy)pyridin-3-yl]-N-[(1R)-1-(2-methylpyrimidin-5-yl)ethyl]-5′,6′-dihydrospiro[pyrrolidine-3,4′-pyrrolo[1,2-b]pyrazole]-1-carboxamide (Mixture of Stereoisomers)

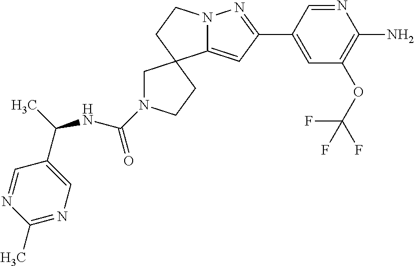 [SMILES: Cc1ncc([C@@H](C)NC(=O)N2CCC3(CCn4nc(-c5cnc(N)c(OC(F)(F)F)c5)cc43)C2)cn1]

Example 238 was prepared in analogy to Example 31 using (rac)-5-[5′,6′-dihydrospiro[pyrrolidine-3,4′-pyrrolo[1,2-b]pyrazol]-2′-yl]-3-(trifluoromethoxy)pyridin-2-amine (75.0 mg, 221 μmol) and (1R)-1-(2-methylpyrimidin-5-yl)ethan-1-amine (36.4 mg, 265 μmol).

LC-MS (method 1): Rt=0.87 min; MS (ESIpos): m/z=503 [M+H]⁺

1H-NMR (400 MHz, DMSO-d6) δ[ppm]: 1.065 (0.44), 1.122 (0.96), 1.138 (0.96), 1.161 (0.91), 1.179 (0.91), 1.241 (0.62), 1.256 (0.93), 1.381 (0.69), 1.401 (5.88), 1.408 (5.97), 1.419 (6.00), 1.425 (5.55), 2.060 (2.41), 2.074 (2.52), 2.154 (0.78), 2.327 (1.05), 2.523 (6.08), 2.544 (3.36), 2.569 (16.00), 2.579 (15.06), 2.622 (0.58), 2.669 (1.18), 3.007 (0.40), 3.174 (0.51), 3.409 (2.23), 3.434 (4.19), 3.440 (3.95), 3.447 (3.66), 3.473 (1.05), 3.483 (0.93), 3.513 (0.93), 3.537 (0.93), 3.553 (0.98), 3.840 (0.45), 4.158 (2.94), 4.176 (5.17), 4.193 (3.48), 4.818 (1.02), 4.837 (1.41), 4.847 (0.94), 6.390 (3.10), 6.401 (6.64), 6.504 (6.44), 6.587 (1.60), 6.599 (1.76), 6.606 (1.76), 6.618 (1.54), 7.051 (0.63), 7.752 (4.14), 7.756 (4.59), 8.324 (3.81), 8.328 (6.17), 8.333 (3.79), 8.351 (1.16), 8.580 (0.74), 8.631 (10.58), 8.656 (10.72).

### Example 239

### 2′-[6-amino-5-(trifluoromethoxy)pyridin-3-yl]-N-[(1R)-1-(pyridin-2-yl)ethyl]-5′,6′-dihydrospiro[pyrrolidine-3,4′-pyrrolo[1,2-b]pyrazole]-1-carboxamide (Mixture of Stereoisomers)

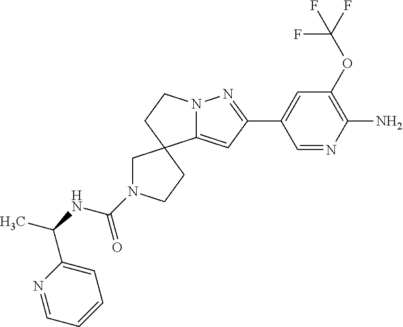 [SMILES: C[C@@H](NC(=O)N1CCC2(CCn3nc(-c4cnc(N)c(OC(F)(F)F)c4)cc32)C1)c1ccccn1]

Example 239 was prepared in analogy to Example 31 using (rac)-5-[5′,6′-dihydrospiro[pyrrolidine-3,4′-pyrrolo[1,2-b]pyrazol]-2′-yl]-3-(trifluoromethoxy)pyridin-2-amine (75.0 mg, 221 μmol) and (1R)-1-(pyridin-2-yl)ethan-1-amine (32.4 mg, 265 μmol).

LC-MS (method 1): Rt=0.95 min; MS (ESIpos): m/z=488 [M+H]⁺

¹H-NMR (400 MHz, DMSO-d₆): δ [ppm]=1.39 (dd, 3H), 2.02-2.16 (m, 2H), 2.54 (br t, 2H), 3.43-3.52 (m, 3H), 3.53-3.64 (m, 1H), 4.19 (t, 2H), 4.83-4.94 (m, 1H), 6.39-6.45 (m, 1H), 6.51 (s, 3H), 7.17-7.26 (m, 1H), 7.40 (dd, 1H), 7.69-7.79 (m, 2H), 8.33 (t, 1H), 8.46-8.52 (m, 1H).

### Example 240

### 2′-[6-amino-5-(trifluoromethoxy)pyridin-3-yl]-N-[(1R)-1-phenylethyl]-5′,6′-dihydrospiro[pyrrolidine-3,4′-pyrrolo[1,2-b]pyrazole]-1-carboxamide (Mixture of Stereoisomer)

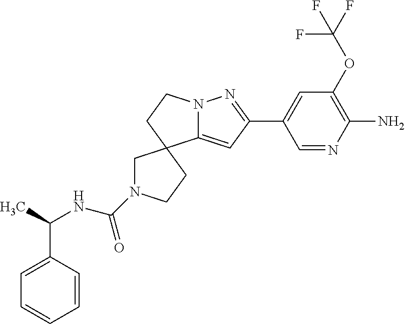 [SMILES: C[C@@H](NC(=O)N1CCC2(CCn3nc(-c4cnc(N)c(OC(F)(F)F)c4)cc32)C1)c1ccccc1]

Example 240 was prepared in analogy to Example 31 using (rac)-5-[5′,6′-dihydrospiro[pyrrolidine-3,4′-pyrrolo[1,2-b]pyrazol]-2′-yl]-3-(trifluoromethoxy)pyridin-2-amine (75.0 mg, 221 μmol) and (1R)-1-phenylethan-1-amine (32.1 mg, 265 μmol).

LC-MS (method 1): Rt=1.09 min; MS (ESIpos): m/z=487 [M+H]⁺

1H-NMR (400 MHz, DMSO-d6) δ[ppm]: 1.018 (0.65), 1.036 (0.88), 1.354 (10.31), 1.360 (11.19), 1.372 (10.68), 1.378 (10.77), 2.061 (4.16), 2.084 (15.58), 2.327 (1.76), 2.523 (10.08), 2.527 (10.17), 2.546 (6.24), 2.669 (1.85), 3.363 (1.43), 3.379 (1.06), 3.427 (3.05), 3.453 (16.00), 3.533 (1.62), 3.556 (1.94), 4.162 (4.90), 4.179 (8.60), 4.196 (4.62), 4.824 (1.85), 4.840 (2.77), 4.858 (2.03), 6.195 (0.46), 6.390 (5.69), 6.407 (11.38), 6.464 (3.14), 6.485 (3.33), 6.505 (12.53), 7.155 (1.16), 7.172 (3.56), 7.189 (4.53), 7.207 (2.13), 7.253 (2.77), 7.273 (6.52), 7.292 (6.24), 7.296 (6.66), 7.315 (10.45), 7.334 (4.67), 7.339 (6.80), 7.360 (3.19), 7.747 (5.27), 8.315 (5.18), 8.320 (6.20), 8.323 (7.12), 8.328 (6.06).

### Example 241

### 2′-[6-amino-5-(trifluoromethoxy)pyridin-3-yl]-N-[(1R)-1-(pyridin-3-yl)ethyl]-5′,6′-dihydrospiro[pyrrolidine-3,4′-pyrrolo[1,2-b]pyrazole]-1-carboxamide (Mixture of Stereoisomers)

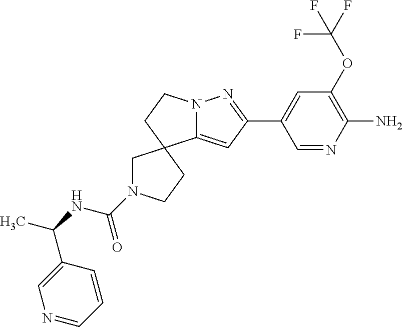 [SMILES: C[C@@H](NC(=O)N1CCC2(CCn3nc(-c4cnc(N)c(OC(F)(F)F)c4)cc32)C1)c1cccnc1]

Example 241 was prepared in analogy to Example 31 using (rac)-5-[5′,6′-dihydrospiro[pyrrolidine-3,4′-pyrrolo[1,2-b]pyrazol]-2′-yl]-3-(trifluoromethoxy)pyridin-2-amine (75.0 mg, 221 μmol) and (1R)-1-(pyridin-3-yl)ethan-1-amine-hydrochloride salt (51.7 mg, 265 μmol).

LC-MS (method 1): Rt=0.91 min; MS (ESIpos): m/z=488 [M+H]⁺

1H-NMR (400 MHz, DMSO-d6) δ[ppm]: 1.101 (1.56), 1.126 (1.38), 1.142 (1.37), 1.165 (1.34), 1.181 (1.35), 1.365 (0.86), 1.388 (12.18), 1.394 (13.05), 1.406 (12.86), 1.412 (12.55), 2.056 (4.78), 2.063 (4.81), 2.071 (4.71), 2.326 (0.58), 2.664 (0.50), 2.668 (0.63), 2.727 (3.61), 2.886 (4.48), 3.404 (2.95), 3.422 (5.39), 3.428 (4.29), 3.448 (11.34), 3.454 (13.21), 3.462 (10.60), 3.481 (3.11), 3.488 (3.13), 3.507 (2.14), 3.525 (2.84), 3.541 (2.64), 3.561 (2.59), 3.576 (1.90), 3.583 (1.73), 3.602 (1.17), 3.724 (0.46), 4.160 (5.68), 4.177 (10.04), 4.194 (5.62), 4.852 (2.02), 4.857 (2.12), 4.870 (3.08), 4.875 (3.03), 4.888 (2.19), 6.395 (6.88), 6.412 (14.46), 6.506 (16.00), 6.569 (3.17), 6.579 (3.61), 6.588 (3.43), 6.599 (3.19), 7.272 (9.12), 7.274 (8.92), 7.291 (2.34), 7.305 (2.55), 7.312 (4.80), 7.324 (4.81), 7.332 (2.75), 7.344 (2.53), 7.714 (1.79), 7.719 (2.85), 7.723 (1.87), 7.734 (1.92), 7.754 (9.07), 7.771 (1.89), 7.950 (0.60), 8.192 (3.36), 8.323 (8.36), 8.325 (10.67), 8.330 (8.50), 8.392 (4.08), 8.396 (4.08), 8.404 (6.73), 8.408 (6.46), 8.417 (3.97), 8.421 (3.59), 8.541 (4.90), 8.547 (4.90), 8.561 (4.80), 8.567 (4.63).

### Example 242

### 2′-[6-amino-5-(trifluoromethoxy)pyridin-3-yl]-N-[(1S)-1-(pyridin-4-yl)ethyl]-5′,6′-dihydrospiro[pyrrolidine-3,4′-pyrrolo[1,2-b]pyrazole]-1-carboxamide (Mixture of Stereoisomers)

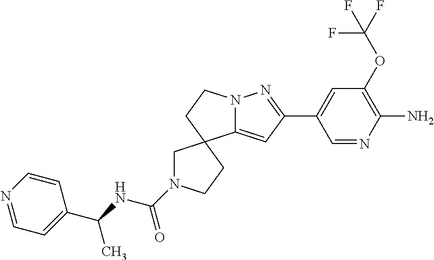 [SMILES: C[C@H](NC(=O)N1CCC2(CCn3nc(-c4cnc(N)c(OC(F)(F)F)c4)cc32)C1)c1ccncc1]

Example 242 was prepared in analogy to Example 31 using (rac)-5-[5′,6′-dihydrospiro[pyrrolidine-3,4′-pyrrolo[1,2-b]pyrazol]-2′-yl]-3-(trifluoromethoxy)pyridin-2-amine (75.0 mg, 221 μmol) and (1S)-1-(pyridin-4-yl)ethan-1-amine (32.4 mg, 265 μmol).

LC-MS (method 2): Rt=0.65 min; MS (ESIpos): m/z=488 [M+H]⁺

1H-NMR (400 MHz, DMSO-d6) δ[ppm]: 1.067 (5.09), 1.141 (1.95), 1.154 (5.30), 1.168 (2.06), 1.185 (1.79), 1.245 (1.65), 1.261 (2.10), 1.267 (2.06), 1.284 (1.56), 1.364 (15.54), 1.378 (15.40), 2.080 (7.06), 2.539 (12.20), 2.556 (7.28), 2.625 (0.68), 3.464 (14.42), 3.478 (13.70), 3.502 (5.24), 3.525 (3.07), 3.542 (3.65), 3.566 (3.53), 3.584 (3.55), 4.169 (6.54), 4.185 (11.09), 4.202 (6.41), 4.810 (3.13), 4.827 (4.46), 4.844 (3.05), 6.423 (8.52), 6.430 (10.27), 6.513 (16.00), 6.614 (3.83), 6.623 (4.48), 6.632 (4.45), 6.641 (3.76), 7.353 (6.70), 7.366 (7.17), 7.384 (6.93), 7.396 (6.72), 7.761 (9.06), 8.137 (1.39), 8.336 (11.55), 8.494 (8.05).

### Example 243

### (rac)-2′-[6-amino-5-(trifluoromethoxy)pyridin-3-yl]-N-[2-(4-fluorophenyl)propan-2-yl]-5′,6′-dihydrospiro[pyrrolidine-3,4′-pyrrolo[1,2-b]pyrazole]-1-carboxamide

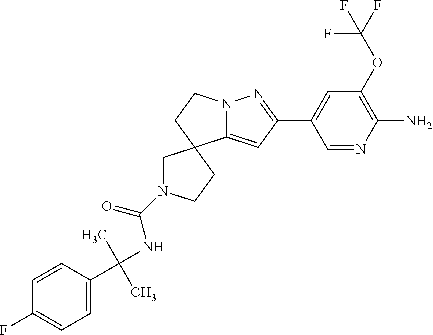 [SMILES: CC(C)(NC(=O)N1CCC2(CCn3nc(-c4cnc(N)c(OC(F)(F)F)c4)cc32)C1)c1ccc(F)cc1]

Example 243 was prepared in analogy to Example 31 using (rac)-5-[5′,6′-dihydrospiro[pyrrolidine-3,4′-pyrrolo[1,2-b]pyrazol]-2′-yl]-3-(trifluoromethoxy)pyridin-2-amine (75.0 mg, 221 μmol) and 2-(4-fluorophenyl)propan-2-amine (40.6 mg, 265 μmol).

LC-MS (method 2): Rt=1.12 min; MS (ESIpos): m/z=519 [M+H]⁺

¹H-NMR (400 MHz, DMSO-d₆): δ [ppm]=1.55 (d, 6H), 1.99-2.13 (m, 2H), 2.52-2.56 (m, 2H), 3.38-3.48 (m, 3H), 3.51-3.61 (m, 1H), 4.19 (t, 2H), 6.07 (s, 1H), 6.41 (s, 1H), 6.51 (s, 2H), 6.99-7.09 (m, 2H), 7.33-7.41 (m, 2H), 7.75 (t, 1H), 8.33 (d, 1H).

### Example 244

### 2′-[6-amino-5-(trifluoromethoxy)pyridin-3-yl]-N-[(1R)-1-(4-fluorophenyl)ethyl]-5′,6′-dihydrospiro[pyrrolidine-3,4′-pyrrolo[1,2-b]pyrazole]-1-carboxamide (Mixture of Stereoisomers)

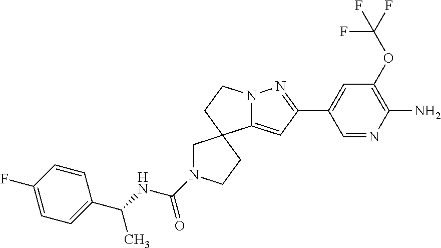 [SMILES: C[C@@H](NC(=O)N1CCC2(CCn3nc(-c4cnc(N)c(OC(F)(F)F)c4)cc32)C1)c1ccc(F)cc1]

Example 244 was prepared in analogy to Example 31 using (rac)-5-[5′,6′-dihydrospiro[pyrrolidine-3,4′-pyrrolo[1,2-b]pyrazol]-2′-yl]-3-(trifluoromethoxy)pyridin-2-amine (75.0 mg, 221 μmol) and (1R)-1-(4-fluorophenyl)ethan-1-amine (36.9 mg, 265 μmol).

LC-MS (method 2): Rt=1.06 min; MS (ESIpos): m/z=505 [M+H]⁺

1H-NMR (400 MHz, DMSO-d6) δ[ppm]: 1.012 (1.72), 1.028 (2.12), 1.044 (0.54), 1.064 (1.39), 1.154 (1.13), 1.228 (0.70), 1.342 (12.14), 1.351 (13.32), 1.360 (13.24), 1.368 (11.59), 1.539 (0.53), 1.556 (0.51), 2.043 (4.14), 2.059 (7.76), 2.073 (4.41), 2.327 (0.85), 2.539 (9.93), 2.555 (6.13), 2.587 (0.53), 2.669 (0.88), 3.385 (1.95), 3.421 (3.00), 3.430 (2.91), 3.447 (14.80), 3.480 (2.78), 3.502 (1.47), 3.519 (2.65), 3.527 (2.22), 3.543 (3.27), 3.559 (2.00), 3.568 (1.85), 3.588 (1.80), 3.679 (0.91), 3.701 (0.99), 3.713 (0.99), 4.007 (3.07), 4.177 (6.72), 4.194 (10.83), 4.211 (6.42), 4.314 (1.04), 4.642 (0.54), 4.820 (2.40), 4.837 (3.53), 4.855 (2.44), 6.494 (16.00), 6.644 (0.77), 7.066 (3.83), 7.089 (11.40), 7.111 (12.69), 7.133 (4.87), 7.159 (0.53), 7.181 (0.69), 7.203 (0.53), 7.231 (1.66), 7.271 (0.54), 7.311 (0.80), 7.337 (4.49), 7.352 (5.64), 7.359 (7.01), 7.376 (6.31), 7.384 (4.97), 7.398 (3.94), 7.506 (0.67), 7.517 (0.64), 7.530 (0.72), 7.551 (0.70), 7.637 (0.46), 7.695 (1.09), 7.995 (5.80), 8.321 (0.51), 8.326 (0.57), 8.345 (10.63), 8.350 (10.32).

### Example 245

### (rac)-2-[6-amino-5-(trifluoromethoxy)pyridin-3-yl]-N-[2-(pyridin-4-yl)propan-2-yl]-6,7-dihydrospiro[pyrazolo[5,1-c][1,4]oxazine-4,3′-pyrrolidine]-1′-carboxamide

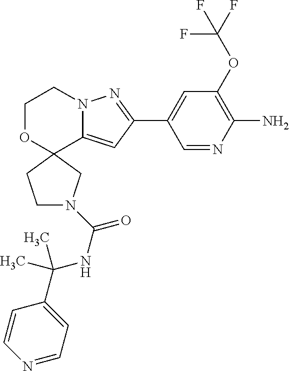 [SMILES: CC(C)(NC(=O)N1CCC2(C1)OCCn1nc(-c3cnc(N)c(OC(F)(F)F)c3)cc12)c1ccncc1]

Example 245 was prepared in analogy to Example 31 using (rac)-5-[6,7-dihydrospiro[pyrazolo[5,1-c][1,4]oxazine-4,3′-pyrrolidin]-2-yl]-3-(trifluoromethoxy)pyridin-2-amine (75.0 mg, 211 μmol) and 2-(pyridin-4-yl)propan-2-amine (34.5 mg, 253 μmol).

LC-MS (method 1): Rₜ=0.94 min; MS (ESIneg): m/z=516 [M−H]⁻

¹H-NMR (400 MHz, DMSO-d6) δ[ppm]: 1.536 (3.38), 2.727 (13.33), 2.886 (16.00), 3.160 (0.98), 3.172 (1.02), 4.104 (0.42), 4.118 (0.73), 4.127 (0.81), 4.137 (0.74), 5.756 (1.44), 6.254 (0.74), 6.561 (1.11), 6.723 (1.27), 7.308 (1.00), 7.313 (0.68), 7.320 (0.69), 7.324 (1.03), 7.749 (0.43), 7.752 (0.58), 7.950 (2.28), 8.330 (0.81), 8.335 (0.84), 8.434 (1.04), 8.438 (0.67), 8.446 (0.67), 8.449 (1.01).

### Example 246

### (rac)-2-[6-amino-5-(trifluoromethoxy)pyridin-3-yl]-N-[1-(pyridin-4-yl)cyclobutyl]-6,7-dihydrospiro[pyrazolo[5,1-c][1,4]oxazine-4,3′-pyrrolidine]-1′-carboxamide

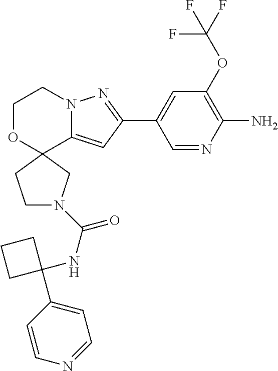 [SMILES: Nc1ncc(-c2cc3n(n2)CCOC32CCN(C(=O)NC3(c4ccncc4)CCC3)C2)cc1OC(F)(F)F]

Example 246 was prepared in analogy to Example 31 using (rac)-5-[6,7-dihydrospiro[pyrazolo[5,1-c][1,4]oxazine-4,3′-pyrrolidin]-2-yl]-3-(trifluoromethoxy)pyridin-2-amine (75.0 mg, 211 μmol) and 1-(pyridin-4-yl)cyclobutan-1-amine (37.5 mg, 253 μmol).

LC-MS (method 1): Rₜ=0.96 min; MS (ESIneg): m/z=528 [M−H]⁻

¹H-NMR (400 MHz, DMSO-d6) δ[ppm]: 1.035 (0.61), 1.052 (1.47), 1.065 (13.73), 1.070 (1.50), 1.170 (0.65), 1.185 (0.67), 1.199 (0.79), 1.215 (1.34), 1.231 (2.12), 1.252 (11.23), 1.269 (16.00), 1.282 (10.47), 1.286 (4.86), 1.298 (10.00), 1.816 (0.43), 1.821 (0.46), 1.838 (0.48), 1.843 (0.54), 1.907 (0.62), 2.009 (0.51), 2.031 (0.47), 2.036 (0.45), 2.244 (0.51), 2.323 (0.68), 2.327 (0.71), 2.332 (0.67), 2.337 (0.73), 2.360 (0.72), 2.373 (0.89), 2.389 (1.01), 2.416 (1.39), 2.434 (1.41), 2.456 (1.00), 2.523 (1.39), 2.540 (0.57), 3.087 (0.43), 3.098 (0.48), 3.105 (1.36), 3.115 (1.35), 3.124 (1.39), 3.134 (1.32), 3.142 (0.52), 3.153 (0.54), 3.164 (1.01), 3.426 (0.48), 3.468 (0.54), 3.496 (0.56), 3.565 (7.41), 3.577 (1.20), 3.587 (1.16), 3.594 (1.59), 3.603 (1.58), 3.610 (1.11), 3.620 (1.09), 3.770 (0.72), 3.799 (0.62), 4.071 (0.60), 4.089 (0.73), 4.121 (2.15), 4.134 (2.87), 6.561 (3.59), 6.718 (3.36), 6.917 (2.02), 7.412 (3.55), 7.417 (2.32), 7.424 (2.38), 7.428 (3.54), 7.739 (1.51), 7.743 (2.01), 8.320 (3.09), 8.324 (2.97), 8.489 (3.68), 8.493 (2.35), 8.501 (2.32), 8.505 (3.50).

### Example 247

### 2′-[6-amino-5-(difluoromethoxy)pyridin-3-yl]-N-(1-phenylcyclobutyl)-5′,6′-dihydrospiro[azetidine-3,4′-pyrrolo[1,2-b]pyrazole]-1-carboxamide

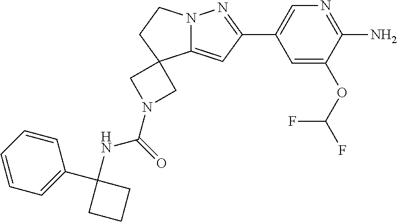 [SMILES: Nc1ncc(-c2cc3n(n2)CCC32CN(C(=O)NC3(c4ccccc4)CCC3)C2)cc1OC(F)F]

Example 247 was prepared in analogy to Example 31 using 3-(difluoromethoxy)-5-(5′,6′-dihydrospiro[azetidine-3,4′-pyrrolo[1,2-b]pyrazol]-2′-yl)pyridin-2-amine-hydrochloride salt (75.0 mg, 218 μmol) and 1-phenylcyclobutan-1-amine-hydrochloride salt (44.1 mg, 240 μmol).

LC-MS (method 1): Rₜ=1.07 min; MS (ESIpos): m/z=480 [M+H]⁺

¹H-NMR (400 MHz, DMSO-d6) δ[ppm]: 1.053 (0.43), 1.066 (6.06), 1.753 (0.53), 1.769 (1.05), 1.775 (1.08), 1.791 (1.11), 1.797 (1.29), 1.813 (0.81), 1.982 (0.65), 1.998 (1.21), 2.021 (1.14), 2.025 (1.08), 2.074 (0.78), 2.084 (0.48), 2.364 (1.02), 2.395 (2.50), 2.411 (2.98), 2.416 (2.55), 2.433 (1.85), 2.448 (2.13), 2.465 (2.54), 2.471 (3.08), 2.523 (4.41), 2.728 (1.44), 2.812 (3.06), 2.829 (5.13), 2.847 (3.37), 2.888 (1.74), 3.315 (1.05), 3.319 (1.25), 3.363 (1.62), 3.942 (1.05), 3.968 (2.23), 3.989 (12.81), 3.994 (12.52), 4.015 (2.27), 4.095 (3.42), 4.113 (5.34), 4.130 (3.31), 5.770 (0.43), 6.164 (8.96), 6.603 (16.00), 6.999 (3.76), 7.070 (7.38), 7.164 (1.03), 7.167 (1.76), 7.170 (1.08), 7.184 (10.45), 7.201 (1.80), 7.204 (2.90), 7.207 (1.60), 7.296 (4.69), 7.315 (7.77), 7.329 (1.78), 7.334 (4.77), 7.368 (3.36), 7.415 (7.24), 7.418 (7.87), 7.436 (6.06), 7.439 (4.64), 7.643 (5.10), 8.236 (8.83), 8.241 (7.98).

### Example 248

### (rac)-2′-[6-amino-5-(trifluoromethoxy)pyridin-3-yl]-N-[2-(pyridin-2-yl)propan-2-yl]-5′,6′-dihydrospiro[pyrrolidine-3,4′-pyrrolo[1,2-b]pyrazole]-1-carboxamide

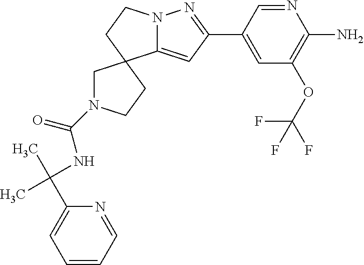 [SMILES: CC(C)(NC(=O)N1CCC2(CCn3nc(-c4cnc(N)c(OC(F)(F)F)c4)cc32)C1)c1ccccn1]

Example 248 was prepared in analogy to Example 31 using (rac)-5-[5′,6′-dihydrospiro[pyrrolidine-3,4′-pyrrolo[1,2-b]pyrazol]-2′-yl]-3-(trifluoromethoxy)pyridin-2-amine (75.0 mg, 221 μmol) and 2-(pyridin-2-yl)propan-2-amine (36.1 mg, 265 μmol).

LC-MS (method 1): Rt=1.02 min; MS (ESIpos): m/z=502 [M+H]⁺

¹H-NMR (400 MHz, DMSO-d₆): δ [ppm]=1.59 (s, 6H), 2.02-2.16 (m, 2H), 2.55 (br t, 2H), 3.41-3.51 (m, 3H), 3.54-3.64 (m, 1H), 4.19 (t, 2H), 6.40-6.46 (m, 2H), 6.51 (s, 2H), 7.19 (dd, 1H), 7.47 (d, 1H), 7.71 (td, 1H), 7.74-7.78 (m, 1H), 8.34 (d, 1H), 8.43-8.49 (m, 1H).

### Example 249

### (rac)-2′-[6-amino-5-(trifluoromethoxy)pyridin-3-yl]-N-[2-(3-chloropyridin-4-yl)propan-2-yl]-5′,6′-dihydrospiro[pyrrolidine-3,4′-pyrrolo[1,2-b]pyrazole]-1-carboxamide

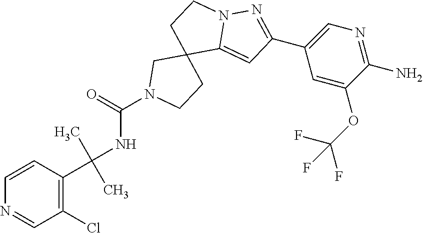 [SMILES: CC(C)(NC(=O)N1CCC2(CCn3nc(-c4cnc(N)c(OC(F)(F)F)c4)cc32)C1)c1ccncc1Cl]

Example 249 was prepared in analogy to Example 31 using (rac)-5-[5′,6′-dihydrospiro[pyrrolidine-3,4′-pyrrolo[1,2-b]pyrazol]-2′-yl]-3-(trifluoromethoxy)pyridin-2-amine (75.0 mg, 221 μmol) and 2-(3-chloropyridin-4-yl)propan-2-amine (45.3 mg, 265 μmol).

LC-MS (method 1): Rₜ=1.02 min; MS (ESIpos): m/z=536 [M+H]⁺

¹H-NMR (400 MHz, DMSO-d6) δ[ppm]: 0.997 (0.75), 1.014 (0.75), 1.627 (1.43), 1.653 (16.00), 2.032 (0.66), 2.046 (1.23), 2.064 (1.43), 2.081 (0.59), 2.518 (3.42), 2.522 (2.58), 3.296 (0.47), 3.348 (1.89), 3.363 (0.41), 3.376 (0.48), 3.380 (0.41), 3.384 (0.40), 3.413 (4.36), 3.500 (0.65), 4.158 (1.56), 4.176 (3.31), 4.194 (1.52), 6.332 (3.14), 6.391 (6.61), 6.514 (4.24), 7.426 (2.66), 7.439 (2.70), 7.735 (1.72), 7.739 (2.33), 7.743 (1.63), 8.319 (3.80), 8.323 (3.93), 8.388 (3.84), 8.402 (3.59), 8.409 (7.15).

### Example 250

### 2′-[6-amino-5-(difluoromethoxy)pyridin-3-yl]-N-[2-(2-fluorophenyl)propan-2-yl]-5′,6′-dihydrospiro[azetidine-3,4′-pyrrolo[1,2-b]pyrazole]-1-carboxamide

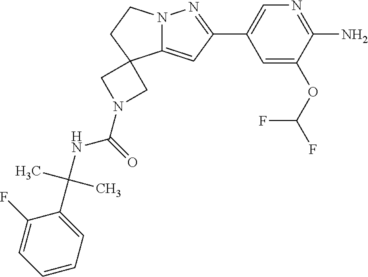 [SMILES: CC(C)(NC(=O)N1CC2(CCn3nc(-c4cnc(N)c(OC(F)F)c4)cc32)C1)c1ccccc1F]

Example 250 was prepared in analogy to Example 31 using 3-(difluoromethoxy)-5-(5′,6′-dihydrospiro[azetidine-3,4′-pyrrolo[1,2-b]pyrazol]-2′-yl)pyridin-2-amine-hydrochloride salt (75.0 mg, 218 μmol) and 2-(2-fluorophenyl)propan-2-amine-hydrochloride salt (45.5 mg, 240 μmol).

LC-MS (method 1): Rₜ=1.08 min; MS (ESIneg): m/z=485 [M−H]⁻

¹H-NMR (400 MHz, DMSO-d6) δ[ppm]: 1.066 (16.00), 1.625 (8.69), 1.665 (2.88), 2.522 (0.41), 2.727 (2.33), 2.819 (0.84), 2.837 (1.39), 2.854 (0.91), 2.887 (2.85), 3.944 (2.27), 4.009 (5.89), 4.102 (0.92), 4.120 (1.48), 4.137 (0.91), 6.167 (2.44), 6.606 (4.65), 6.611 (2.24), 7.007 (0.93), 7.058 (0.41), 7.062 (0.45), 7.079 (0.52), 7.082 (0.57), 7.093 (0.47), 7.104 (0.48), 7.107 (0.52), 7.114 (0.65), 7.122 (1.05), 7.126 (0.85), 7.142 (0.73), 7.145 (0.61), 7.191 (1.92), 7.231 (0.45), 7.238 (0.42), 7.245 (0.41), 7.250 (0.42), 7.309 (0.68), 7.313 (0.47), 7.331 (0.80), 7.375 (0.83), 7.652 (1.37), 8.248 (2.15), 8.253 (2.06).

### Example 251

### (rac)-2-[6-amino-5-(difluoromethoxy)pyridin-3-yl]-N-[2-(3-fluorophenyl)propan-2-yl]-6,7-dihydrospiro[pyrazolo[5,1-c][1,4]oxazine-4,3′-pyrrolidine]-1′-carboxamide

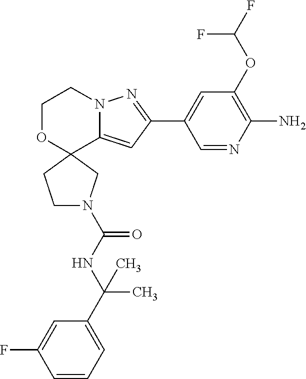 [SMILES: CC(C)(NC(=O)N1CCC2(C1)OCCn1nc(-c3cnc(N)c(OC(F)F)c3)cc12)c1cccc(F)c1]

Example 251 was prepared in analogy to Example 31 using (rac)-3-(difluoromethoxy)-5-[6,7-dihydrospiro[pyrazolo[5,1-c][1,4]oxazine-4,3′-pyrrolidin]-2-yl]pyridin-2-amine-hydrochloride salt (75.0 mg, 201 μmol) and 2-(3-fluorophenyl)propan-2-amine (36.9 mg, 241 μmol).

LC-MS (method 1): Rₜ=1.08 min; MS (ESIpos): m/z=517 [M+H]⁺

¹H-NMR (400 MHz, DMSO-d6) δ[ppm]: 1.137 (0.72), 1.232 (0.86), 1.494 (1.28), 1.549 (16.00), 1.555 (15.49), 2.226 (0.70), 2.251 (0.96), 2.275 (0.53), 2.309 (0.85), 2.323 (2.30), 2.327 (2.94), 2.332 (2.02), 2.336 (1.06), 2.357 (0.45), 2.518 (6.15), 2.523 (4.30), 2.660 (0.65), 2.665 (1.44), 2.669 (2.07), 2.673 (1.46), 2.678 (0.61), 3.366 (0.42), 3.391 (0.82), 3.408 (0.84), 3.482 (1.08), 3.511 (1.28), 3.575 (0.67), 3.597 (1.17), 3.621 (0.54), 3.788 (1.48), 3.816 (1.27), 4.070 (0.43), 4.084 (0.94), 4.101 (1.63), 4.123 (5.31), 4.132 (4.10), 4.149 (1.06), 6.148 (4.92), 6.217 (6.77), 6.680 (10.42), 6.941 (0.87), 6.945 (0.95), 6.947 (0.97), 6.960 (1.65), 6.967 (1.80), 6.982 (0.97), 6.987 (1.09), 6.989 (1.08), 6.994 (3.11), 7.105 (1.33), 7.111 (1.71), 7.116 (1.40), 7.133 (1.27), 7.138 (1.74), 7.144 (1.39), 7.179 (8.00), 7.197 (2.53), 7.276 (1.58), 7.292 (1.78), 7.297 (2.48), 7.312 (2.37), 7.316 (1.25), 7.332 (1.02), 7.363 (2.58), 7.617 (3.76), 7.619 (3.60), 8.198 (6.84), 8.203 (6.16).

### Example 252

### 2-[6-amino-5-(difluoromethoxy)pyridin-3-yl]-N-[(1S)-1-(3-chloropyridin-4-yl)ethyl]-6,7-dihydrospiro[pyrazolo[5,1-c][1,4]oxazine-4,3′-pyrrolidine]-1′-carboxamide (Mixture of Stereoisomers)

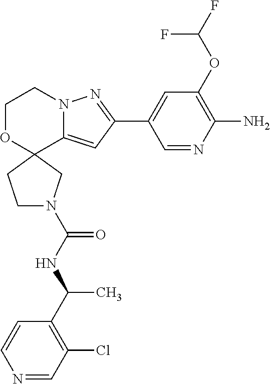 [SMILES: C[C@H](NC(=O)N1CCC2(C1)OCCn1nc(-c3cnc(N)c(OC(F)F)c3)cc12)c1ccncc1Cl]

Example 252 was prepared in analogy to Example 31 using (rac)-3-(difluoromethoxy)-5-[6,7-dihydrospiro[pyrazolo[5,1-c][1,4]oxazine-4,3′-pyrrolidin]-2-yl]pyridin-2-amine-hydrochloride salt (75.0 mg, 201 μmol) and (1S)-1-(3-chloropyridin-4-yl)ethan-1-amine-hydrochloride salt (46.5 mg, 241 μmol).

LC-MS (method 1): Rₜ=0.90 min; MS (ESIpos): m/z=520 [M+H]⁺

¹H-NMR (400 MHz, DMSO-d6) δ[ppm]: 1.041 (0.44), 1.060 (0.48), 1.137 (0.67), 1.232 (1.01), 1.335 (8.93), 1.337 (9.20), 1.352 (9.24), 1.355 (9.12), 2.084 (16.00), 2.235 (0.82), 2.262 (0.99), 2.332 (2.50), 2.336 (1.65), 2.348 (1.11), 2.518 (8.14), 2.523 (5.73), 2.673 (1.91), 2.678 (0.83), 3.370 (0.49), 3.393 (0.74), 3.410 (0.91), 3.432 (0.89), 3.458 (1.10), 3.489 (0.85), 3.527 (0.72), 3.556 (1.14), 3.585 (0.95), 3.610 (0.43), 3.646 (0.54), 3.668 (0.92), 3.690 (0.43), 3.776 (1.23), 3.803 (1.02), 3.842 (1.22), 3.870 (1.06), 4.050 (0.52), 4.064 (1.08), 4.080 (1.55), 4.097 (2.10), 4.121 (4.37), 4.132 (5.74), 5.046 (1.10), 5.055 (1.30), 5.064 (1.77), 5.073 (1.88), 5.082 (1.26), 5.090 (1.18), 6.221 (7.84), 6.673 (5.21), 6.681 (6.04), 6.774 (1.45), 6.793 (1.46), 6.810 (1.43), 6.828 (1.34), 6.993 (2.08), 6.998 (2.23), 7.178 (4.52), 7.183 (4.64), 7.362 (1.95), 7.367 (1.91), 7.483 (3.12), 7.495 (3.61), 7.499 (3.50), 7.511 (3.04), 7.615 (2.93), 7.620 (2.92), 8.193 (4.55), 8.197 (4.47), 8.203 (5.02), 8.208 (4.85), 8.483 (4.11), 8.494 (5.27), 8.506 (4.10), 8.534 (14.65).

### Example 253

### 2-[6-amino-5-(trifluoromethoxy)pyridin-3-yl]-N-[1-(1,3,5-trimethyl-1H-pyrazol-4-yl)ethyl]-6,7-dihydrospiro[pyrazolo[5,1-c][1,4]oxazine-4,3′-pyrrolidine]-1′-carboxamide (Mixture of Stereoisomers)

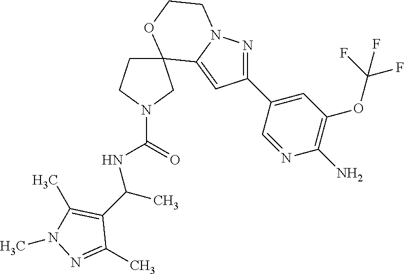 [SMILES: Cc1nn(C)c(C)c1C(C)NC(=O)N1CCC2(C1)OCCn1nc(-c3cnc(N)c(OC(F)(F)F)c3)cc12]

Example 253 was prepared in analogy to Example 31 using (rac)-5-[6,7-dihydrospiro[pyrazolo[5,1-c][1,4]oxazine-4,3′-pyrrolidin]-2-yl]-3-(trifluoromethoxy)pyridin-2-amine (75.0 mg, 211 μmol) and 1-(1,3,5-trimethyl-1H-pyrazol-4-yl)ethan-1-amine (38.8 mg, 253 μmol).

LC-MS (method 1): Rₜ=0.92 min; MS (ESIneg): m/z=533 [M−H]⁻

¹H-NMR (400 MHz, DMSO-d6) δ[ppm]: 1.066 (4.69), 1.109 (0.88), 1.125 (0.91), 1.137 (0.88), 1.154 (0.89), 1.285 (0.45), 1.304 (0.51), 1.327 (3.63), 1.333 (3.82), 1.345 (3.75), 1.351 (3.72), 2.096 (1.22), 2.120 (13.83), 2.169 (1.47), 2.191 (16.00), 2.213 (0.80), 2.238 (0.42), 2.288 (0.62), 2.304 (0.74), 2.322 (0.56), 2.327 (0.49), 2.332 (0.44), 2.336 (0.44), 2.518 (0.69), 2.523 (0.45), 2.728 (0.74), 2.888 (0.84), 3.318 (0.63), 3.357 (0.55), 3.374 (0.47), 3.384 (1.18), 3.413 (1.02), 3.435 (0.70), 3.464 (0.81), 3.523 (0.62), 3.563 (15.13), 3.578 (0.78), 3.724 (0.67), 3.751 (0.54), 3.797 (0.65), 3.824 (0.58), 3.940 (0.80), 4.052 (0.68), 4.059 (0.75), 4.077 (0.76), 4.090 (1.11), 4.098 (1.28), 4.118 (3.14), 4.682 (0.71), 4.686 (0.72), 4.700 (1.01), 4.703 (1.04), 4.717 (0.70), 4.720 (0.68), 6.086 (0.82), 6.103 (0.80), 6.134 (0.87), 6.150 (0.84), 6.554 (4.73), 6.677 (3.88), 6.694 (3.09), 7.734 (1.29), 7.738 (2.26), 7.742 (2.19), 8.312 (2.16), 8.317 (3.94), 8.321 (2.11).

### Example 254

### 2-[6-amino-5-(trifluoromethoxy)pyridin-3-yl]-N-[1-(1-ethyl-3,5-dimethyl-1H-pyrazol-4-yl)ethyl]-6,7-dihydrospiro[pyrazolo[5,1-c][1,4]oxazine-4,3′-pyrrolidine]-1′-carboxamide (Mixture of Stereoisomers)

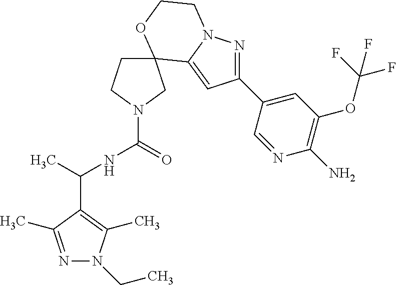 [SMILES: CCn1nc(C)c(C(C)NC(=O)N2CCC3(C2)OCCn2nc(-c4cnc(N)c(OC(F)(F)F)c4)cc23)c1C]

Example 254 was prepared in analogy to Example 31 using (rac)-5-[6,7-dihydrospiro[pyrazolo[5,1-c][1,4]oxazine-4,3′-pyrrolidin]-2-yl]-3-(trifluoromethoxy)pyridin-2-amine (75.0 mg, 211 μmol) and 1-(1-ethyl-3,5-dimethyl-1H-pyrazol-4-yl)ethan-1-amine (42.4 mg, 253 μmol).

LC-MS (method 1): Rₜ=0.99 min; MS (ESIpos): m/z=549 [M+H]⁺

¹H-NMR (400 MHz, DMSO-d6) δ[ppm]: 1.209 (4.01), 1.227 (8.63), 1.244 (4.19), 1.331 (3.79), 1.337 (4.16), 1.349 (3.94), 1.355 (3.96), 2.143 (16.00), 2.179 (0.73), 2.186 (0.89), 2.207 (15.83), 2.238 (0.47), 2.291 (0.69), 2.306 (0.83), 2.323 (0.65), 2.327 (0.62), 2.337 (0.47), 2.523 (0.60), 2.540 (0.91), 3.317 (0.62), 3.326 (0.87), 3.421 (2.24), 3.438 (1.28), 3.468 (1.08), 3.509 (0.46), 3.532 (0.72), 3.559 (0.54), 3.584 (0.69), 3.731 (0.72), 3.760 (0.60), 3.798 (0.75), 3.827 (0.66), 3.874 (1.16), 3.892 (3.55), 3.910 (3.48), 3.928 (1.11), 4.043 (0.61), 4.054 (0.80), 4.060 (0.89), 4.078 (0.91), 4.091 (1.22), 4.100 (1.42), 4.119 (3.56), 4.700 (0.67), 4.706 (0.74), 4.717 (1.00), 4.723 (1.05), 4.735 (0.70), 4.741 (0.69), 6.090 (0.84), 6.107 (0.83), 6.139 (0.88), 6.156 (0.85), 6.576 (1.44), 6.681 (3.96), 6.698 (3.09), 7.740 (1.42), 7.744 (2.47), 7.747 (2.47), 8.311 (2.27), 8.316 (4.41), 8.321 (2.50).

### Example 255

### 2-[6-amino-5-(trifluoromethoxy)pyridin-3-yl]-N-[(1R)-1-(2-cyanophenyl)ethyl]-6,7-dihydrospiro[pyrazolo[5,1-c][1,4]oxazine-4,3′-pyrrolidine]-1′-carboxamide (Mixture of Stereoisomers)

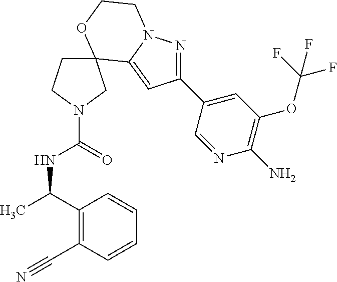 [SMILES: C[C@@H](NC(=O)N1CCC2(C1)OCCn1nc(-c3cnc(N)c(OC(F)(F)F)c3)cc12)c1ccccc1C#N]

Example 255 was prepared in analogy to Example 31 using (rac)-5-[6,7-dihydrospiro[pyrazolo[5,1-c][1,4]oxazine-4,3′-pyrrolidin]-2-yl]-3-(trifluoromethoxy)pyridin-2-amine (75.0 mg, 211 μmol) and 2-[(1R)-1-aminoethyl]benzonitrile (37.0 mg, 253 μmol).

LC-MS (method 1): Rₜ=1.04 min; MS (ESIpos): m/z=528 [M+H]⁺

¹H-NMR (400 MHz, DMSO-d6) δ[ppm]: 1.067 (0.89), 1.170 (0.74), 1.185 (0.75), 1.199 (0.85), 1.215 (1.08), 1.227 (1.64), 1.392 (15.91), 1.410 (16.00), 2.197 (0.49), 2.221 (1.48), 2.247 (1.75), 2.272 (0.80), 2.332 (1.84), 2.346 (1.89), 2.518 (1.04), 2.523 (0.73), 2.539 (0.85), 2.996 (0.44), 3.167 (0.98), 3.392 (1.48), 3.410 (1.70), 3.419 (1.79), 3.436 (1.60), 3.462 (1.66), 3.492 (1.53), 3.502 (1.73), 3.532 (1.70), 3.565 (2.19), 3.587 (1.48), 3.613 (0.77), 3.626 (0.99), 3.648 (1.65), 3.672 (0.76), 3.779 (1.88), 3.807 (1.64), 3.826 (2.09), 3.856 (1.75), 4.044 (0.72), 4.058 (1.54), 4.074 (2.46), 4.093 (2.91), 4.105 (3.70), 4.112 (4.33), 4.124 (5.97), 4.131 (7.71), 4.136 (9.05), 5.021 (0.66), 5.039 (2.91), 5.057 (4.59), 5.074 (3.01), 5.092 (0.67), 6.559 (12.87), 6.711 (12.86), 6.767 (2.48), 6.784 (2.67), 6.793 (2.61), 6.810 (2.24), 7.379 (1.76), 7.383 (3.11), 7.386 (1.94), 7.398 (3.38), 7.402 (5.83), 7.405 (3.50), 7.416 (2.21), 7.420 (3.63), 7.424 (2.21), 7.621 (2.07), 7.629 (2.15), 7.638 (4.06), 7.646 (4.11), 7.663 (2.39), 7.667 (2.59), 7.673 (2.64), 7.676 (2.88), 7.685 (2.87), 7.691 (2.76), 7.694 (2.96), 7.701 (1.18), 7.705 (1.18), 7.711 (1.12), 7.714 (1.16), 7.744 (11.31), 7.754 (5.39), 7.758 (3.71), 7.762 (6.79), 8.319 (6.49), 8.324 (6.54), 8.329 (7.65), 8.334 (6.98).

### Example 256

### (rac)-2′-[6-amino-5-(difluoromethoxy)pyridin-3-yl]-N-[2-(pyridin-2-yl)propan-2-yl]-5′,6′-dihydrospiro[pyrrolidine-3,4′-pyrrolo[1,2-b]pyrazole]-1-carboxamide

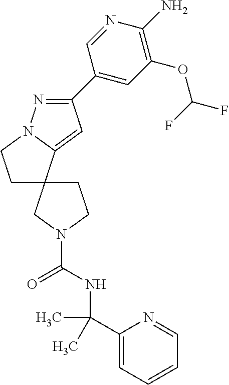 [SMILES: CC(C)(NC(=O)N1CCC2(CCn3nc(-c4cnc(N)c(OC(F)F)c4)cc32)C1)c1ccccn1]

Example 256 was prepared in analogy to Example 31 using (rac)-3-(difluoromethoxy)-5-[5′,6′-dihydrospiro[pyrrolidine-3,4′-pyrrolo[1,2-b]pyrazol]-2′-yl]pyridin-2-amine-hydrochloride salt (130 mg, 330 μmol) and 2-(pyridin-2-yl)propan-2-amine (53.9 mg, 396 μmol).

LC-MS (method 1): Rt=0.95 min; MS (ESIpos): m/z=484 [M+H]⁺

¹H-NMR (400 MHz, DMSO-d₆): δ [ppm]=1.60 (s, 6H), 2.01-2.16 (m, 2H), 2.53-2.59 (m, 2H), 3.43-3.50 (m, 4H), 3.54-3.63 (m, 1H), 4.19 (t, 2H), 6.33 (br d, 1H), 6.41 (s, 1H), 6.47 (s, 1H), 7.00-7.40 (m, 1H), 7.28 (br d, 1H), 7.54 (br d, 1H), 7.66 (s, 1H), 7.74-7.87 (m, 1H), 8.20 (d, 1H), 8.49 (d, 1H).

### Example 257

### 2-[6-amino-5-(trifluoromethoxy)pyridin-3-yl]-N-[(1R)-1-(4-fluorophenyl)ethyl]-6,7-dihydrospiro[pyrazolo[5,1-c][1,4]oxazine-4,3′-pyrrolidine]-1′-carboxamide (Mixture of Stereoisomers)

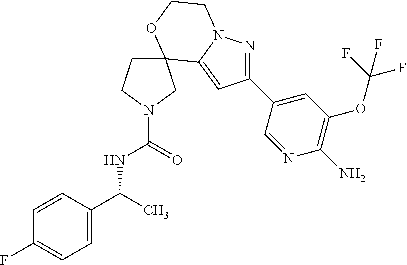 [SMILES: C[C@@H](NC(=O)N1CCC2(C1)OCCn1nc(-c3cnc(N)c(OC(F)(F)F)c3)cc12)c1ccc(F)cc1]

Example 257 was prepared in analogy to Example 31 using (rac)-5-[6,7-dihydrospiro[pyrazolo[5,1-c][1,4]oxazine-4,3′-pyrrolidin]-2-yl]-3-(trifluoromethoxy)pyridin-2-amine (75.0 mg, 211 μmol) and (1R)-1-(4-fluorophenyl)ethan-1-amine (35.3 mg, 253 μmol).

LC-MS (method 1): Rₜ=1.10 min; MS (ESIpos): m/z=521 [M+H]⁺

¹H-NMR (400 MHz, DMSO-d6) δ[ppm]: 1.243 (0.62), 1.248 (0.41), 1.260 (0.74), 1.278 (0.52), 1.348 (3.32), 1.366 (3.26), 1.472 (0.62), 1.489 (0.64), 2.084 (16.00), 2.322 (0.61), 2.326 (0.71), 2.332 (0.64), 2.518 (1.59), 2.522 (0.99), 2.669 (0.40), 3.372 (0.60), 3.388 (0.63), 3.397 (0.79), 3.577 (0.67), 3.610 (0.57), 3.772 (0.43), 3.798 (0.74), 4.068 (0.43), 4.075 (0.42), 4.114 (0.84), 4.128 (1.89), 4.832 (0.55), 6.495 (0.50), 6.506 (0.53), 6.515 (0.54), 6.526 (0.47), 6.712 (2.82), 6.991 (0.48), 7.090 (0.75), 7.096 (0.95), 7.112 (1.75), 7.118 (2.35), 7.135 (1.08), 7.140 (0.92), 7.246 (0.48), 7.288 (0.44), 7.349 (1.14), 7.363 (1.34), 7.370 (1.20), 7.385 (0.96), 7.763 (1.13), 7.768 (1.09), 8.314 (1.37), 8.319 (2.36), 8.324 (1.37).

### Example 258

### 2-[6-amino-5-(trifluoromethoxy)pyridin-3-yl]-N-[(1R)-1-phenylpropyl]-6,7-dihydrospiro[pyrazolo[5,1-c][1,4]oxazine-4,3′-pyrrolidine]-1′-carboxamide (Mixture of Stereoisomers)

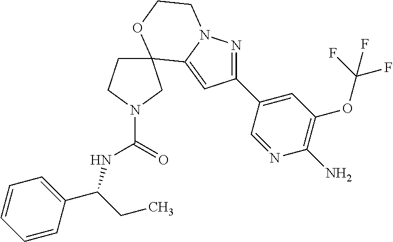 [SMILES: CC[C@@H](NC(=O)N1CCC2(C1)OCCn1nc(-c3cnc(N)c(OC(F)(F)F)c3)cc12)c1ccccc1]

Example 258 was prepared in analogy to Example 31 using (rac)-5-[6,7-dihydrospiro[pyrazolo[5,1-c][1,4]oxazine-4,3′-pyrrolidin]-2-yl]-3-(trifluoromethoxy)pyridin-2-amine (75.0 mg, 211 μmol) and (1R)-1-phenylpropan-1-amine (34.2 mg, 253 μmol).

LC-MS (method 1): Rₜ=1.15 min; MS (ESIpos): m/z=517 [M+H]⁺

¹H-NMR (400 MHz, DMSO-d6) δ[ppm]: 0.742 (1.15), 0.761 (2.66), 0.779 (1.28), 0.836 (5.54), 0.851 (12.66), 0.854 (11.95), 0.869 (6.27), 1.066 (1.85), 1.239 (1.24), 1.255 (1.13), 1.625 (0.80), 1.642 (1.46), 1.658 (2.52), 1.675 (2.76), 1.694 (2.33), 1.717 (2.22), 1.736 (1.69), 1.752 (1.21), 2.213 (1.49), 2.240 (1.99), 2.263 (1.12), 2.327 (3.67), 2.331 (3.64), 2.523 (8.62), 2.665 (1.61), 2.669 (2.18), 3.236 (0.55), 3.277 (0.74), 3.290 (1.35), 3.302 (1.25), 3.381 (2.87), 3.392 (3.09), 3.413 (1.65), 3.435 (1.01), 3.454 (1.97), 3.484 (3.87), 3.513 (2.20), 3.552 (0.87), 3.575 (1.70), 3.597 (1.55), 3.617 (1.64), 3.764 (1.94), 3.798 (2.31), 3.830 (1.55), 4.125 (8.42), 4.536 (1.01), 4.557 (2.47), 4.577 (2.37), 4.596 (1.11), 6.414 (2.68), 6.436 (2.76), 6.556 (10.52), 6.699 (7.45), 6.706 (9.77), 7.170 (1.33), 7.188 (3.58), 7.205 (2.68), 7.271 (3.03), 7.287 (7.46), 7.291 (7.47), 7.309 (16.00), 7.330 (3.90), 7.443 (5.61), 7.737 (6.17), 7.741 (5.93), 8.229 (0.55), 8.309 (5.61), 8.315 (10.71), 8.320 (6.12).

### Example 259

### 2-[6-amino-5-(trifluoromethoxy)pyridin-3-yl]-N-[(1R)-1-phenylethyl]-6,7-dihydrospiro[pyrazolo[5,1-c][1,4]oxazine-4,3′-pyrrolidine]-1′-carboxamide (Mixture of Stereoisomers)

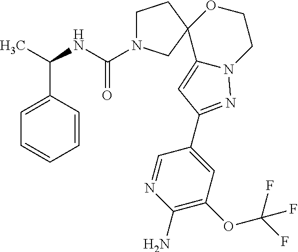 [SMILES: C[C@@H](NC(=O)N1CCC2(C1)OCCn1nc(-c3cnc(N)c(OC(F)(F)F)c3)cc12)c1ccccc1]

Example 259 was prepared in analogy to Example 31 using (rac)-5-[6,7-dihydrospiro[pyrazolo[5,1-c][1,4]oxazine-4,3′-pyrrolidin]-2-yl]-3-(trifluoromethoxy)pyridin-2-amine (75.0 mg, 211 μmol) and (1R)-1-phenylethan-1-amine (30.7 mg, 253 μmol).

LC-MS (method 1): Rₜ=1.10 min; MS (ESIpos): m/z=503 [M+H]⁺

¹H-NMR (400 MHz, DMSO-d6) δ[ppm]: 0.760 (0.44), 1.019 (0.63), 1.036 (0.81), 1.066 (2.30), 1.225 (1.51), 1.241 (5.98), 1.257 (10.08), 1.274 (5.13), 1.358 (15.82), 1.376 (16.00), 1.479 (1.11), 1.496 (1.11), 2.084 (1.05), 2.211 (1.32), 2.237 (1.64), 2.323 (2.89), 2.327 (3.52), 2.331 (3.14), 2.518 (14.44), 2.523 (10.15), 2.665 (1.62), 2.669 (2.11), 2.673 (1.64), 2.729 (0.42), 3.122 (0.69), 3.133 (0.70), 3.141 (0.71), 3.152 (0.77), 3.397 (3.16), 3.457 (2.00), 3.472 (2.32), 3.487 (2.28), 3.501 (2.56), 3.566 (0.98), 3.590 (2.31), 3.614 (2.32), 3.636 (1.04), 3.787 (1.94), 3.800 (2.03), 3.813 (1.78), 3.828 (1.72), 4.068 (2.00), 4.103 (3.23), 4.115 (4.56), 4.129 (9.05), 4.818 (1.93), 4.837 (3.00), 4.856 (2.05), 6.489 (2.99), 6.508 (3.11), 6.567 (5.47), 6.704 (8.70), 6.708 (10.99), 6.730 (0.59), 6.969 (0.48), 7.097 (0.58), 7.173 (1.54), 7.191 (4.08), 7.208 (2.90), 7.224 (0.56), 7.273 (2.78), 7.278 (3.46), 7.293 (7.48), 7.298 (7.70), 7.311 (6.03), 7.316 (5.87), 7.329 (11.77), 7.347 (4.87), 7.445 (1.40), 7.469 (0.66), 7.745 (5.16), 7.781 (0.55), 8.222 (1.14), 8.228 (1.24), 8.315 (6.28), 8.321 (8.35), 8.326 (6.81).

### Example 260

### (rac)-2-[6-amino-5-(trifluoromethoxy)pyridin-3-yl]-N-[1-(pyridin-3-yl)cyclobutyl]-6,7-dihydrospiro[pyrazolo[5,1-c][1,4]oxazine-4,3′-pyrrolidine]-1′-carboxamide

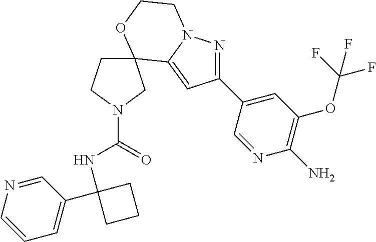 [SMILES: Nc1ncc(-c2cc3n(n2)CCOC32CCN(C(=O)NC3(c4cccnc4)CCC3)C2)cc1OC(F)(F)F]

Example 260 was prepared in analogy to Example 31 using (rac)-5-[6,7-dihydrospiro[pyrazolo[5,1-c][1,4]oxazine-4,3′-pyrrolidin]-2-yl]-3-(trifluoromethoxy)pyridin-2-amine (75.0 mg, 211 μmol) and 1-(pyridin-3-yl)cyclobutan-1-amine (37.5 mg, 253 μmol).

LC-MS (method 1): Rt=0.98 min; MS (ESIpos): m/z=530 [M+H]⁺

¹H-NMR (400 MHz, DMSO-d6) δ[ppm]: 0.851 (0.92), 1.035 (4.80), 1.052 (10.51), 1.065 (6.04), 1.070 (4.99), 1.114 (1.05), 1.130 (1.39), 1.170 (4.69), 1.185 (4.93), 1.199 (5.53), 1.215 (7.04), 1.231 (7.57), 1.333 (0.62), 1.788 (1.92), 1.810 (2.33), 1.828 (1.56), 2.011 (2.12), 2.034 (2.05), 2.208 (1.64), 2.234 (2.10), 2.256 (1.18), 2.327 (3.89), 2.449 (7.70), 2.471 (8.16), 2.518 (11.76), 2.523 (7.86), 2.540 (3.49), 2.669 (1.73), 2.729 (1.12), 2.888 (1.34), 3.159 (4.11), 3.172 (4.22), 3.236 (0.56), 3.256 (0.62), 3.273 (0.45), 3.280 (0.60), 3.292 (1.49), 3.319 (3.49), 3.382 (7.05), 3.405 (2.17), 3.410 (2.16), 3.417 (1.92), 3.422 (2.53), 3.429 (1.89), 3.435 (2.67), 3.440 (2.81), 3.452 (3.80), 3.481 (2.60), 3.514 (0.68), 3.557 (1.64), 3.565 (1.63), 3.578 (2.88), 3.600 (1.37), 3.751 (3.44), 3.780 (2.87), 3.946 (0.70), 4.046 (1.15), 4.061 (2.29), 4.076 (3.39), 4.111 (6.70), 4.125 (11.62), 4.353 (0.83), 4.366 (1.72), 4.379 (0.92), 4.780 (0.56), 4.860 (0.58), 4.876 (0.82), 4.891 (0.63), 6.555 (16.00), 6.706 (14.55), 6.836 (8.58), 7.305 (4.40), 7.306 (4.55), 7.319 (4.58), 7.326 (4.73), 7.336 (4.91), 7.338 (4.75), 7.735 (6.15), 7.738 (8.29), 7.776 (3.91), 7.781 (5.15), 7.787 (4.09), 7.797 (3.59), 7.802 (4.64), 7.807 (3.55), 8.311 (12.50), 8.316 (12.99), 8.373 (7.75), 8.377 (7.81), 8.384 (7.34), 8.389 (7.31), 8.634 (8.86), 8.638 (8.82), 8.883 (0.67), 9.732 (0.90).

### Example 261

### 2-[6-amino-5-(difluoromethoxy)pyridin-3-yl]-N-[(1R)-1-(2-chloro-4-cyanophenyl)ethyl]-6,7-dihydrospiro[pyrazolo[5,1-c][1,4]oxazine-4,3′-pyrrolidine]-1′-carboxamide (Mixture of Stereoisomers)

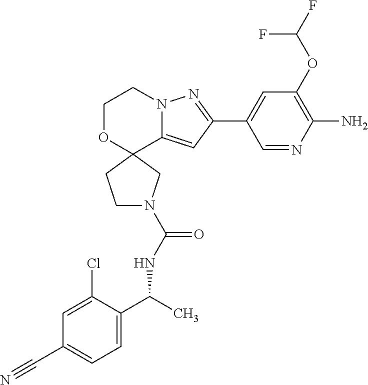 [SMILES: C[C@@H](NC(=O)N1CCC2(C1)OCCn1nc(-c3cnc(N)c(OC(F)F)c3)cc12)c1ccc(C#N)cc1Cl]

Example 261 was prepared in analogy to Example 31 using (rac)-3-(difluoromethoxy)-5-[6,7-dihydrospiro[pyrazolo[5,1-c][1,4]oxazine-4,3′-pyrrolidin]-2-yl]pyridin-2-amine-hydrochloride salt (75.0 mg, 201 μmol) and 4-[(1R)-1-aminoethyl]-3-chlorobenzonitrile (43.5 mg, 241 μmol).

LC-MS (method 1): Rt=1.03 min; MS (ESIpos): m/z=544 [M+H]⁺

¹H-NMR (400 MHz, DMSO-d6) δ[ppm]: 1.232 (1.01), 1.256 (0.54), 1.329 (6.29), 1.346 (6.22), 2.084 (0.41), 2.231 (0.59), 2.258 (0.76), 2.518 (16.00), 2.522 (11.23), 3.381 (0.53), 3.398 (0.61), 3.424 (0.61), 3.446 (0.76), 3.474 (0.58), 3.511 (0.50), 3.542 (0.69), 3.573 (0.64), 3.659 (0.64), 3.768 (0.74), 3.795 (0.66), 3.843 (0.82), 3.872 (0.73), 4.060 (0.67), 4.075 (1.00), 4.092 (1.22), 4.129 (3.69), 5.116 (0.70), 5.125 (0.81), 5.135 (1.15), 5.143 (1.23), 5.153 (0.80), 5.161 (0.79), 5.759 (7.07), 6.221 (5.45), 6.675 (3.28), 6.682 (3.53), 6.781 (0.92), 6.799 (0.91), 6.820 (0.89), 6.839 (0.82), 6.993 (1.29), 6.997 (1.36), 7.177 (2.74), 7.182 (2.83), 7.361 (1.20), 7.366 (1.18), 7.618 (2.05), 7.664 (1.83), 7.677 (1.86), 7.684 (2.26), 7.697 (2.13), 7.825 (1.37), 7.829 (1.46), 7.834 (1.59), 7.838 (1.61), 7.845 (1.20), 7.849 (1.21), 7.855 (1.20), 7.859 (1.21), 7.992 (4.47), 7.996 (4.22), 8.190 (2.76), 8.195 (2.72), 8.201 (3.00), 8.206 (3.02).

### Example 262

### (rac)-2-[6-amino-5-(difluoromethoxy)pyridin-3-yl]-N-[2-(4-fluorophenyl)propan-2-yl]-6,7-dihydrospiro[pyrazolo[5,1-c][1,4]oxazine-4,3′-pyrrolidine]-1′-carboxamide

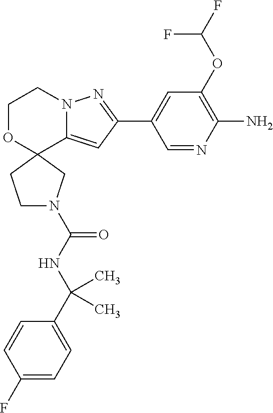 [SMILES: CC(C)(NC(=O)N1CCC2(C1)OCCn1nc(-c3cnc(N)c(OC(F)F)c3)cc12)c1ccc(F)cc1]

Example 262 was prepared in analogy to Example 31 using (rac)-3-(difluoromethoxy)-5-[6,7-dihydrospiro[pyrazolo[5,1-c][1,4]oxazine-4,3′-pyrrolidin]-2-yl]pyridin-2-amine-hydrochloride salt (75.0 mg, 201 μmol) and 2-(4-fluorophenyl)propan-2-amine (36.9 mg, 241 μmol).

LC-MS (method 1): Rt=1.09 min; MS (ESIpos): m/z=517 [M+H]⁺

¹H-NMR (400 MHz, DMSO-d6) δ[ppm]: 1.066 (1.28), 1.232 (0.59), 1.550 (16.00), 1.556 (15.55), 2.074 (3.12), 2.084 (1.43), 2.215 (0.74), 2.240 (1.07), 2.265 (0.56), 2.306 (0.93), 2.323 (2.20), 2.327 (2.31), 2.331 (1.76), 2.337 (1.21), 2.352 (0.56), 2.518 (11.08), 2.523 (8.12), 2.665 (1.18), 2.669 (1.66), 2.673 (1.16), 2.728 (0.54), 2.889 (0.66), 3.381 (1.69), 3.398 (1.87), 3.424 (1.63), 3.499 (2.36), 3.571 (1.15), 3.592 (1.65), 3.615 (0.87), 3.778 (1.68), 3.807 (1.44), 4.067 (0.52), 4.081 (1.08), 4.099 (1.63), 4.124 (4.85), 4.134 (4.44), 6.099 (4.87), 6.385 (0.81), 6.689 (9.59), 7.015 (2.45), 7.049 (3.58), 7.054 (1.21), 7.071 (7.73), 7.088 (1.35), 7.093 (4.05), 7.101 (0.48), 7.198 (5.06), 7.352 (3.77), 7.358 (1.71), 7.366 (4.23), 7.374 (3.93), 7.383 (3.62), 7.388 (3.59), 7.653 (3.50), 8.192 (6.26), 8.197 (6.56).

### Example 263

(rac)-2-[6-amino-5-(difluoromethoxy)pyridin-3-yl]-N-[1-(2-fluorophenyl)cyclobutyl]-6,7-dihydrospiro[pyrazolo[5,1-c][1,4]oxazine-4,3′-pyrrolidine]-1′-carboxamide

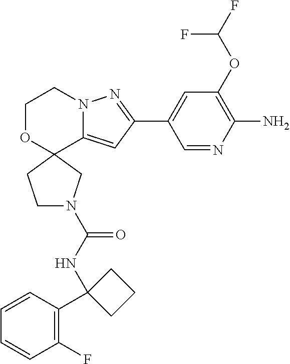 [SMILES: Nc1ncc(-c2cc3n(n2)CCOC32CCN(C(=O)NC3(c4ccccc4F)CCC3)C2)cc1OC(F)F]

Example 263 was prepared in analogy to Example 31 using (rac)-3-(difluoromethoxy)-5-[6,7-dihydrospiro[pyrazolo[5,1-c][1,4]oxazine-4,3′-pyrrolidin]-2-yl]pyridin-2-amine-hydrochloride salt (75.0 mg, 201 μmol) and 1-(2-fluorophenyl)cyclobutan-1-amine-hydrochloride salt (48.6 mg, 241 μmol).

LC-MS (method 1): Rₜ=1.11 min; MS (ESIpos): m/z=529 [M+H]⁺

¹H-NMR (400 MHz, DMSO-d6) δ[ppm]: 1.232 (0.48), 1.728 (0.42), 1.734 (0.43), 1.741 (0.45), 1.755 (0.47), 2.033 (0.53), 2.055 (0.51), 2.060 (0.50), 2.085 (16.00), 2.218 (0.48), 2.285 (0.45), 2.300 (0.54), 2.318 (0.76), 2.323 (1.22), 2.327 (1.61), 2.332 (1.26), 2.336 (0.65), 2.518 (6.63), 2.523 (5.43), 2.660 (0.46), 2.665 (1.03), 2.669 (1.45), 2.673 (1.01), 2.678 (0.43), 3.416 (0.67), 3.445 (0.79), 3.513 (0.43), 3.534 (0.78), 3.727 (0.87), 3.755 (0.75), 4.039 (0.52), 4.055 (0.79), 4.074 (0.79), 4.087 (0.90), 4.095 (1.16), 4.112 (2.54), 6.213 (3.75), 6.592 (2.33), 6.644 (4.04), 6.990 (1.59), 7.043 (0.63), 7.046 (0.72), 7.063 (0.85), 7.066 (0.98), 7.074 (1.75), 7.093 (3.00), 7.111 (1.24), 7.114 (0.94), 7.174 (3.36), 7.198 (0.45), 7.202 (0.51), 7.211 (0.52), 7.216 (0.76), 7.222 (0.64), 7.235 (0.70), 7.359 (1.38), 7.404 (0.68), 7.409 (0.69), 7.428 (1.08), 7.445 (0.61), 7.601 (2.05), 8.174 (3.24), 8.178 (3.35).

### Example 264

### (rac)-2′-[6-amino-5-(difluoromethoxy)pyridin-3-yl]-N-[2-(pyridin-3-yl)propan-2-yl]-5′,6′-dihydrospiro[pyrrolidine-3,4′-pyrrolo[1,2-b]pyrazole]-1-carboxamide

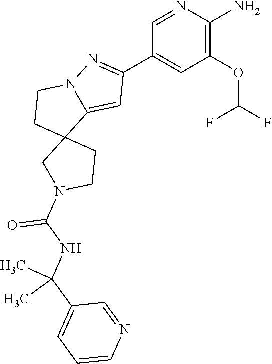 [SMILES: CC(C)(NC(=O)N1CCC2(CCn3nc(-c4cnc(N)c(OC(F)F)c4)cc32)C1)c1cccnc1]

Example 264 was prepared in analogy to Example 31 using (rac)-3-(difluoromethoxy)-5-[5′,6′-dihydrospiro[pyrrolidine-3,4′-pyrrolo[1,2-b]pyrazol]-2′-yl]pyridin-2-amine-hydrochloride salt (130 mg, 330 μmol) and 2-(pyridin-3-yl)propan-2-amine (53.9 mg, 396 μmol).

LC-MS (method 1): Rt=0.89 min; MS (ESIpos): m/z=484 [M+H]⁺

¹H-NMR (400 MHz, DMSO-d₆): δ [ppm]=1.61 (s, 6H), 1.99-2.15 (m, 2H), 2.52-2.57 (m, 2H), 3.40-3.49 (m, 4H), 3.51-3.60 (m, 1H), 4.19 (t, 2H), 6.31-6.42 (m, 2H), 6.42-6.56 (m, 1H), 7.01-7.43 (m, 1H), 7.63-7.71 (m, 2H), 8.15-8.23 (m, 2H), 8.55-8.60 (m, 1H), 8.72 (d, 1H).

### Example 265

### 2′-[6-amino-5-(difluoromethoxy)pyridin-3-yl]-N-[1-(2-chlorophenyl)cyclobutyl]-5′,6′-dihydrospiro[azetidine-3,4′-pyrrolo[1,2-b]pyrazole]-1-carboxamide

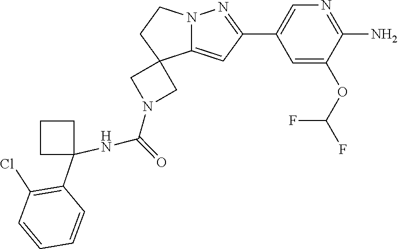 [SMILES: Nc1ncc(-c2cc3n(n2)CCC32CN(C(=O)NC3(c4ccccc4Cl)CCC3)C2)cc1OC(F)F]

Example 265 was prepared in analogy to Example 31 using 3-(difluoromethoxy)-5-(5′,6′-dihydrospiro[azetidine-3,4′-pyrrolo[1,2-b]pyrazol]-2′-yl)pyridin-2-amine (75.0 mg, 244 μmol) and 1-(2-chlorophenyl)cyclobutan-1-amine-hydrochloride salt (63.9 mg, 293 μmol).

LC-MS (method 1): Rₜ=1.17 min; MS (ESIpos): m/z=515 [M+H]⁺

¹H-NMR (400 MHz, DMSO-d6) δ[ppm]: 1.208 (2.07), 1.216 (0.50), 1.224 (2.19), 1.241 (0.90), 1.248 (1.61), 1.259 (2.04), 1.265 (1.95), 1.273 (1.80), 1.277 (1.37), 1.289 (1.50), 1.661 (0.40), 1.672 (0.69), 1.683 (1.03), 1.694 (1.00), 1.698 (1.04), 1.710 (1.16), 1.720 (0.85), 1.731 (0.49), 2.030 (0.83), 2.051 (1.51), 2.073 (1.45), 2.077 (1.47), 2.099 (0.88), 2.322 (0.43), 2.327 (0.62), 2.332 (0.48), 2.470 (1.19), 2.518 (6.02), 2.522 (5.30), 2.543 (1.44), 2.596 (1.66), 2.606 (1.95), 2.618 (2.22), 2.628 (2.83), 2.649 (1.18), 2.660 (1.12), 2.669 (0.81), 2.673 (0.56), 2.727 (0.54), 2.774 (3.23), 2.791 (5.24), 2.808 (3.38), 2.887 (0.57), 3.928 (1.70), 3.951 (16.00), 3.972 (1.67), 4.019 (0.56), 4.074 (3.37), 4.092 (5.42), 4.109 (3.32), 6.160 (8.39), 6.537 (15.50), 6.543 (2.13), 6.994 (3.62), 7.017 (7.32), 7.046 (0.41), 7.179 (7.56), 7.193 (1.42), 7.197 (1.46), 7.211 (3.39), 7.216 (3.29), 7.230 (3.19), 7.235 (3.22), 7.241 (2.63), 7.245 (3.15), 7.260 (3.41), 7.264 (3.86), 7.278 (1.71), 7.282 (1.48), 7.317 (4.96), 7.321 (4.86), 7.336 (3.21), 7.340 (3.24), 7.363 (3.06), 7.453 (0.62), 7.462 (0.43), 7.524 (3.39), 7.528 (3.29), 7.543 (3.10), 7.547 (2.84), 7.629 (5.09), 7.631 (4.98), 8.214 (7.65), 8.219 (7.93).

### Example 266

### 2′-[6-amino-5-(trifluoromethoxy)pyridin-3-yl]-N-[(1R)-1-(4-cyanophenyl)ethyl]-5′,6′-dihydrospiro[azetidine-3,4′-pyrrolo[1,2-b]pyrazole]-1-carboxamide

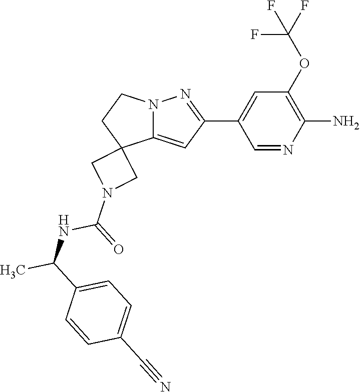 [SMILES: C[C@@H](NC(=O)N1CC2(CCn3nc(-c4cnc(N)c(OC(F)(F)F)c4)cc32)C1)c1ccc(C#N)cc1]

Example 266 was prepared in analogy to Example 31 using 5-(5′,6′-dihydrospiro[azetidine-3,4′-pyrrolo[1,2-b]pyrazol]-2′-yl)-3-(trifluoromethoxy)pyridin-2-amine-hydrochloride salt (75.0 mg, 207 μmol) and 4-[(1R)-1-aminoethyl]benzonitrile (36.4 mg, 249 μmol).

LC-MS (method 1): Rₜ=1.03 min; MS (ESIpos): m/z=498 [M+H]⁺

¹H-NMR (400 MHz, DMSO-d6) δ[ppm]: 1.035 (1.35), 1.052 (3.02), 1.070 (1.39), 1.224 (0.64), 1.354 (10.97), 1.371 (11.03), 1.824 (0.92), 2.518 (0.87), 2.523 (0.60), 2.827 (2.97), 2.845 (4.93), 2.862 (3.19), 2.884 (0.45), 3.167 (11.97), 3.430 (1.13), 3.447 (0.88), 3.464 (0.42), 3.563 (0.51), 4.003 (2.66), 4.029 (10.49), 4.050 (5.45), 4.071 (2.53), 4.104 (3.44), 4.122 (5.37), 4.139 (3.30), 4.835 (1.61), 4.853 (2.32), 4.872 (1.58), 6.504 (8.86), 6.675 (16.00), 7.018 (3.65), 7.038 (3.53), 7.522 (7.74), 7.542 (9.03), 7.770 (3.53), 7.775 (5.01), 7.779 (4.00), 7.785 (11.00), 7.790 (3.32), 7.802 (2.97), 7.806 (8.61), 8.366 (7.98), 8.371 (7.64).

### Example 267

### 2′-[6-amino-5-(difluoromethoxy)pyridin-3-yl]-N-[(1R)-1-(1,3,4-trimethyl-1H-pyrazol-5-yl)ethyl]-5′,6′-dihydrospiro[azetidine-3,4′-pyrrolo[1,2-b]pyrazole]-1-carboxamide

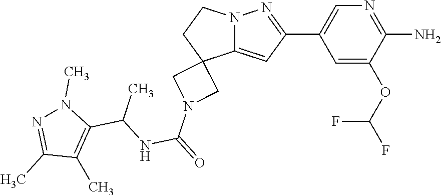 [SMILES: Cc1nn(C)c(C(C)NC(=O)N2CC3(CCn4nc(-c5cnc(N)c(OC(F)F)c5)cc43)C2)c1C]

Example 267 was prepared in analogy to Example 31 using 3-(difluoromethoxy)-5-(5′,6′-dihydrospiro[azetidine-3,4′-pyrrolo[1,2-b]pyrazol]-2′-yl)pyridin-2-amine-hydrochloride salt (75.0 mg, 218 μmol) and 1-(1,3,4-trimethyl-1H-pyrazol-5-yl)ethan-1-amine (40.1 mg, 262 μmol).

LC-MS (method 1): Rₜ=0.89 min; MS (ESIpos): m/z=487 [M+H]⁺

¹H-NMR (400 MHz, DMSO-d6) δ[ppm]: 1.066 (8.53), 1.156 (2.86), 1.387 (5.86), 1.406 (5.87), 1.795 (0.42), 1.926 (0.44), 1.957 (15.99), 1.969 (0.73), 1.986 (14.44), 2.518 (2.28), 2.523 (1.51), 2.807 (1.46), 2.824 (2.48), 2.842 (1.56), 3.682 (0.42), 3.713 (16.00), 3.939 (1.35), 3.951 (1.47), 3.972 (2.74), 3.989 (2.69), 4.003 (2.88), 4.012 (2.45), 4.024 (1.12), 4.033 (1.37), 4.091 (1.63), 4.109 (2.62), 4.125 (1.55), 4.860 (1.00), 4.878 (1.44), 4.896 (1.00), 6.162 (4.33), 6.596 (7.27), 6.895 (1.79), 6.911 (1.70), 6.995 (1.59), 7.180 (3.32), 7.364 (1.45), 7.634 (2.45), 8.226 (3.71), 8.231 (3.70).

### Example 268

### (rac)-2′-[6-amino-5-(trifluoromethoxy)pyridin-3-yl]-N-[1-(2-chlorophenyl)cyclobutyl]-5′,6′-dihydrospiro[pyrrolidine-3,4′-pyrrolo[1,2-b]pyrazole]-1-carboxamide

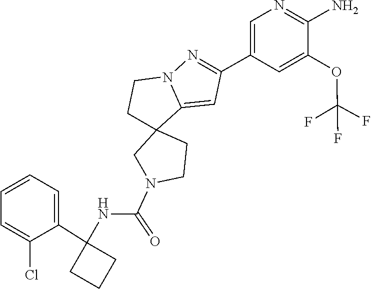 [SMILES: Nc1ncc(-c2cc3n(n2)CCC32CCN(C(=O)NC3(c4ccccc4Cl)CCC3)C2)cc1OC(F)(F)F]

Example 268 was prepared in analogy to Example 31 using (rac)-5-[5′,6′-dihydrospiro[pyrrolidine-3,4′-pyrrolo[1,2-b]pyrazol]-2′-yl]-3-(trifluoromethoxy)pyridin-2-amine-hydrochloride salt (75.0 mg, 200 μmol) and 1-(2-chlorophenyl)cyclobutan-1-amine (43.5 mg, 239 μmol).

LC-MS (method 1): Rt=1.26 min; MS (ESIpos): m/z=547 [M+H]⁺

¹H-NMR (400 MHz, DMSO-d6) δ [ppm]: 0.917 (0.99), 0.932 (8.27), 0.934 (2.71), 0.948 (8.54), 0.952 (1.44), 1.232 (0.95), 1.649 (0.70), 1.660 (1.13), 1.671 (1.70), 1.686 (1.66), 1.698 (1.78), 1.708 (1.32), 1.720 (0.81), 1.998 (2.15), 2.012 (3.86), 2.028 (5.66), 2.048 (3.74), 2.074 (2.65), 2.084 (0.92), 2.096 (1.40), 2.116 (0.42), 2.322 (1.09), 2.327 (1.57), 2.332 (1.11), 2.404 (0.82), 2.423 (0.93), 2.459 (3.22), 2.476 (8.12), 2.518 (7.91), 2.523 (6.64), 2.551 (2.81), 2.572 (1.29), 2.597 (1.70), 2.608 (1.99), 2.620 (3.02), 2.628 (2.76), 2.649 (2.45), 2.660 (2.67), 2.664 (2.15), 2.669 (2.95), 2.673 (1.93), 2.679 (1.44), 2.941 (0.47), 2.957 (0.65), 2.974 (0.49), 3.345 (3.18), 3.347 (2.93), 3.374 (6.12), 3.392 (7.16), 3.418 (2.24), 3.445 (1.34), 3.464 (2.33), 3.478 (1.67), 3.488 (1.51), 3.504 (0.81), 4.137 (4.88), 4.155 (9.90), 4.173 (4.72), 6.295 (9.98), 6.511 (16.00), 6.521 (11.11), 7.160 (2.08), 7.164 (2.38), 7.178 (5.52), 7.183 (5.56), 7.197 (5.27), 7.202 (4.98), 7.226 (3.93), 7.230 (5.11), 7.245 (5.50), 7.249 (6.72), 7.263 (2.99), 7.267 (2.81), 7.280 (8.52), 7.283 (7.26), 7.298 (5.82), 7.302 (5.30), 7.528 (5.50), 7.532 (5.61), 7.547 (5.12), 7.552 (4.85), 7.711 (5.47), 7.715 (7.61), 7.719 (5.22), 8.281 (12.38), 8.286 (11.77).

### Example 269

### 2′-[6-amino-5-(difluoromethoxy)pyridin-3-yl]-N-[(1R)-1-(5-fluoropyridin-3-yl)ethyl]-5′,6′-dihydrospiro[azetidine-3,4′-pyrrolo[1,2-b]pyrazole]-1-carboxamide

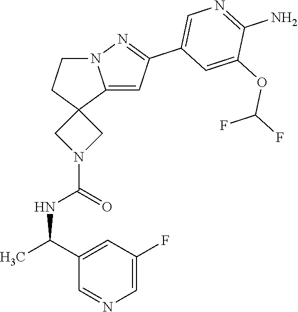 [SMILES: C[C@@H](NC(=O)N1CC2(CCn3nc(-c4cnc(N)c(OC(F)F)c4)cc32)C1)c1cncc(F)c1]

Example 269 was prepared in analogy to Example 31 using 3-(difluoromethoxy)-5-(5′,6′-dihydrospiro[azetidine-3,4′-pyrrolo[1,2-b]pyrazol]-2′-yl)pyridin-2-amine (75.0 mg, 244 μmol) and (1R)-1-(5-fluoropyridin-3-yl)ethan-1-amine (41.0 mg, 293 μmol).

LC-MS (method 1): Rₜ=0.87 min; MS (ESIpos): m/z=474 [M+H]⁺

¹H-NMR (400 MHz, DMSO-d6) δ [ppm]: 0.798 (0.54), 0.815 (0.58), 0.822 (0.58), 0.905 (0.67), 1.035 (8.33), 1.053 (16.00), 1.070 (8.13), 1.390 (3.11), 1.408 (3.12), 2.518 (1.89), 2.523 (1.22), 2.829 (0.78), 2.846 (1.27), 2.864 (0.85), 3.404 (1.05), 3.418 (1.13), 3.422 (3.58), 3.435 (3.66), 3.440 (3.33), 3.452 (3.39), 3.457 (1.19), 3.469 (1.13), 4.002 (0.74), 4.022 (1.40), 4.035 (3.20), 4.058 (1.30), 4.078 (0.75), 4.100 (0.91), 4.117 (1.44), 4.135 (0.86), 4.343 (2.34), 4.356 (4.53), 4.369 (2.18), 4.876 (0.40), 4.894 (0.58), 4.914 (0.41), 5.759 (0.90), 6.164 (2.32), 6.630 (4.50), 6.974 (0.97), 6.996 (1.50), 7.181 (2.06), 7.365 (0.84), 7.636 (1.42), 7.641 (1.31), 7.656 (0.44), 7.662 (0.59), 7.667 (0.47), 7.682 (0.43), 7.687 (0.57), 7.693 (0.45), 8.230 (2.16), 8.234 (2.23), 8.439 (1.73), 8.446 (2.34), 8.451 (1.70), 8.456 (0.97).

### Example 270

### 2′-[6-amino-5-(trifluoromethoxy)pyridin-3-yl]-N-[(1R)-1-(4-fluorophenyl)ethyl]-5′,6′-dihydrospiro[azetidine-3,4′-pyrrolo[1,2-b]pyrazole]-1-carboxamide

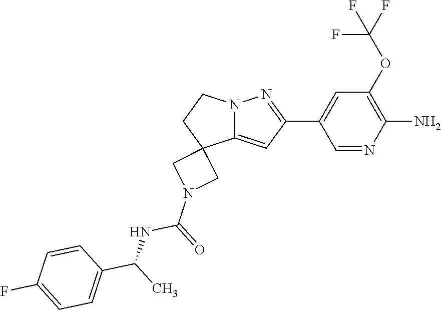 [SMILES: C[C@@H](NC(=O)N1CC2(CCn3nc(-c4cnc(N)c(OC(F)(F)F)c4)cc32)C1)c1ccc(F)cc1]

Example 270 was prepared in analogy to Example 31 using 5-(5′,6′-dihydrospiro[azetidine-3,4′-pyrrolo[1,2-b]pyrazol]-2′-yl)-3-(trifluoromethoxy)pyridin-2-amine-hydrochloride salt (75.0 mg, 207 μmol) and (1R)-1-(4-fluorophenyl)ethan-1-amine (34.6 mg, 249 μmol).

LC-MS (method 1): Rₜ=1.10 min; MS (ESIpos): m/z=491 [M+H]⁺

¹H-NMR (400 MHz, DMSO-d6) δ [ppm]: 0.833 (0.44), 0.850 (0.58), 1.153 (0.85), 1.171 (1.61), 1.189 (0.88), 1.231 (3.15), 1.339 (11.89), 1.357 (11.97), 1.986 (3.01), 2.083 (0.97), 2.518 (11.96), 2.523 (8.33), 2.819 (3.10), 2.836 (5.17), 2.853 (3.43), 3.159 (0.78), 3.171 (0.93), 3.295 (0.73), 3.300 (0.86), 3.306 (0.99), 3.310 (0.82), 3.329 (3.13), 3.391 (2.84), 3.401 (1.52), 3.413 (1.29), 3.421 (0.92), 3.433 (0.62), 3.443 (0.57), 3.458 (0.42), 3.985 (2.38), 4.008 (14.82), 4.023 (6.04), 4.034 (1.32), 4.044 (2.31), 4.100 (3.40), 4.118 (5.45), 4.134 (3.36), 4.784 (1.53), 4.802 (2.12), 4.821 (1.54), 6.502 (9.14), 6.664 (16.00), 6.884 (3.81), 6.905 (3.63), 7.112 (4.71), 7.117 (1.64), 7.129 (2.03), 7.134 (9.60), 7.140 (2.08), 7.151 (1.81), 7.157 (5.43), 7.164 (0.71), 7.348 (4.63), 7.353 (2.01), 7.362 (5.19), 7.369 (4.55), 7.377 (1.78), 7.383 (3.93), 7.764 (3.60), 7.769 (4.87), 7.773 (3.43), 8.359 (8.11), 8.363 (7.84).

### Example 271

### 2′-[6-amino-5-(trifluoromethoxy)pyridin-3-yl]-N-[(1R)-1-(2-chloro-4-cyanophenyl)ethyl]-5′,6′-dihydrospiro[pyrrolidine-3,4′-pyrrolo[1,2-b]pyrazole]-1-carboxamide (Mixture of Stereoisomers)

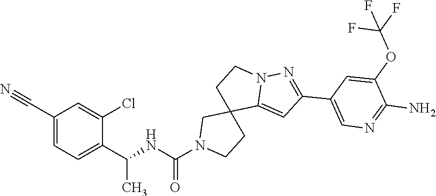 [SMILES: C[C@@H](NC(=O)N1CCC2(CCn3nc(-c4cnc(N)c(OC(F)(F)F)c4)cc32)C1)c1ccc(C#N)cc1Cl]

Example 271 was prepared in analogy to Example 31 using (rac)-5-[5′,6′-dihydrospiro[pyrrolidine-3,4′-pyrrolo[1,2-b]pyrazol]-2′-yl]-3-(trifluoromethoxy)pyridin-2-amine (75.0 mg, 221 μmol) and 4-[(1R)-1-aminoethyl]-3-chlorobenzonitrile (47.9 mg, 265 μmol).

LC-MS (method 1): Rₜ=1.11 min; MS (ESIpos): m/z=546 [M+H]⁺

¹H-NMR (400 MHz, DMSO-d6) δ [ppm]: 1.066 (0.59), 1.323 (8.06), 1.330 (8.78), 1.341 (8.65), 1.347 (8.71), 1.465 (2.43), 2.069 (3.06), 2.073 (3.09), 2.081 (3.35), 2.523 (3.91), 2.532 (6.25), 2.550 (3.71), 2.729 (0.52), 2.886 (0.59), 3.403 (0.58), 3.427 (2.72), 3.452 (4.57), 3.492 (3.77), 3.509 (2.16), 3.516 (2.11), 3.542 (0.94), 3.571 (0.60), 3.590 (0.85), 4.165 (2.67), 4.171 (3.00), 4.184 (4.85), 4.188 (5.35), 4.200 (3.03), 4.205 (2.64), 5.111 (0.49), 5.128 (2.15), 5.146 (3.38), 5.164 (2.24), 5.181 (0.54), 6.425 (16.00), 6.505 (11.92), 6.775 (1.98), 6.792 (3.37), 6.809 (1.96), 7.647 (2.69), 7.667 (3.47), 7.703 (2.68), 7.723 (3.72), 7.757 (5.84), 7.779 (2.32), 7.782 (2.30), 7.799 (1.76), 7.803 (1.92), 7.808 (3.05), 7.812 (2.96), 7.828 (1.92), 7.832 (2.03), 7.979 (5.78), 7.983 (8.82), 7.988 (5.23), 8.334 (8.94), 8.339 (8.59).

### Example 272

### 2′-[6-amino-5-(difluoromethoxy)pyridin-3-yl]-N-[(1R)-1-(2-cyanophenyl)ethyl]-5′,6′-dihydrospiro[azetidine-3,4′-pyrrolo[1,2-b]pyrazole]-1-carboxamide

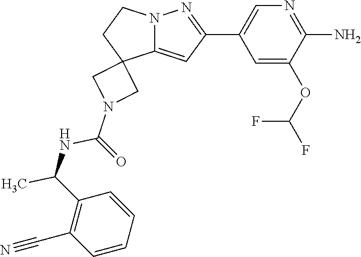 [SMILES: C[C@@H](NC(=O)N1CC2(CCn3nc(-c4cnc(N)c(OC(F)F)c4)cc32)C1)c1ccccc1C#N]

Example 272 was prepared in analogy to Example 31 using 3-(difluoromethoxy)-5-(5′,6′-dihydrospiro[azetidine-3,4′-pyrrolo[1,2-b]pyrazol]-2′-yl)pyridin-2-amine (75.0 mg, 244 μmol) and 2-[(1R)-1-aminoethyl]benzonitrile (42.8 mg, 293 μmol).

LC-MS (method 1): Rₜ=0.96 min; MS (ESIpos): m/z=480 [M+H]⁺

¹H-NMR (400 MHz, DMSO-d6) δ [ppm]: 1.014 (0.47), 1.031 (0.96), 1.047 (0.58), 1.050 (0.57), 1.325 (0.50), 1.342 (0.53), 1.383 (11.62), 1.401 (11.64), 2.074 (1.88), 2.518 (3.41), 2.523 (2.17), 2.827 (2.96), 2.845 (4.89), 2.862 (3.19), 3.361 (0.41), 3.810 (0.42), 4.014 (2.75), 4.036 (8.12), 4.042 (7.29), 4.062 (5.72), 4.083 (2.53), 4.102 (3.40), 4.120 (5.31), 4.137 (3.26), 4.992 (0.41), 5.010 (1.87), 5.028 (2.88), 5.045 (1.86), 5.063 (0.42), 5.768 (1.53), 6.147 (1.02), 6.167 (8.94), 6.424 (0.56), 6.618 (16.00), 6.998 (3.71), 7.165 (3.69), 7.183 (11.18), 7.200 (0.74), 7.367 (3.26), 7.385 (0.51), 7.405 (2.00), 7.408 (2.01), 7.424 (3.86), 7.426 (4.00), 7.442 (2.39), 7.445 (2.44), 7.556 (0.41), 7.609 (2.89), 7.629 (4.57), 7.639 (5.13), 7.641 (5.06), 7.698 (2.24), 7.701 (2.52), 7.717 (3.04), 7.720 (3.36), 7.736 (1.38), 7.739 (1.50), 7.763 (3.94), 7.765 (3.78), 7.782 (3.55), 7.785 (3.16), 8.079 (0.60), 8.084 (0.62), 8.231 (8.24), 8.235 (8.56).

### Example 273

### 2′-[6-amino-5-(difluoromethoxy)pyridin-3-yl]-N-[2-(pyridin-4-yl)propan-2-yl]-5′,6′-dihydrospiro[azetidine-3,4′-pyrrolo[1,2-b]pyrazole]-1-carboxamide

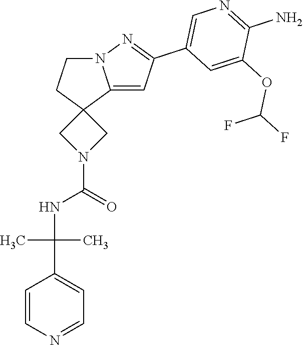 [SMILES: CC(C)(NC(=O)N1CC2(CCn3nc(-c4cnc(N)c(OC(F)F)c4)cc32)C1)c1ccncc1]

Example 273 was prepared in analogy to Example 31 using 3-(difluoromethoxy)-5-(5′,6′-dihydrospiro[azetidine-3,4′-pyrrolo[1,2-b]pyrazol]-2′-yl)pyridin-2-amine (75.0 mg, 244 μmol) and 2-(pyridin-4-yl)propan-2-amine (39.9 mg, 293 μmol).

LC-MS (method 1): Rₜ=0.87 min; MS (ESIpos): m/z=470 [M+H]⁺

¹H-NMR (400 MHz, DMSO-d6) δ [ppm]: 1.230 (0.40), 1.531 (16.00), 1.871 (0.57), 2.518 (0.81), 2.522 (0.52), 2.814 (0.65), 2.829 (1.26), 2.846 (2.08), 2.863 (1.35), 3.166 (1.55), 3.910 (0.56), 4.005 (0.66), 4.027 (6.19), 4.049 (0.73), 4.106 (1.46), 4.124 (2.34), 4.141 (1.44), 6.150 (0.47), 6.175 (3.61), 6.440 (0.66), 6.631 (6.54), 6.713 (2.96), 7.008 (1.48), 7.193 (3.10), 7.320 (3.75), 7.324 (2.40), 7.331 (2.45), 7.336 (3.79), 7.377 (1.38), 7.655 (2.01), 7.657 (1.98), 8.253 (3.33), 8.258 (3.24), 8.463 (4.01), 8.468 (2.32), 8.475 (2.36), 8.479 (3.70).

### Example 274

### 2′-[6-amino-5-(difluoromethoxy)pyridin-3-yl]-N-[(1R)-1-(4-fluorophenyl)ethyl]-5′,6′-dihydrospiro[azetidine-3,4′-pyrrolo[1,2-b]pyrazole]-1-carboxamide

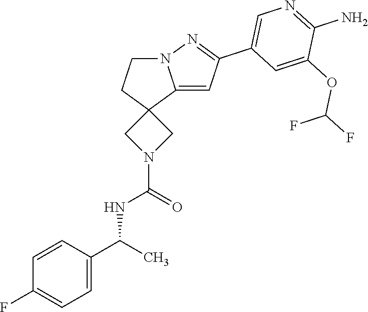 [SMILES: C[C@@H](NC(=O)N1CC2(CCn3nc(-c4cnc(N)c(OC(F)F)c4)cc32)C1)c1ccc(F)cc1]

Example 274 was prepared in analogy to Example 31 using 3-(difluoromethoxy)-5-(5′,6′-dihydrospiro[azetidine-3,4′-pyrrolo[1,2-b]pyrazol]-2′-yl)pyridin-2-amine (75.0 mg, 244 μmol) and (1R)-1-(4-fluorophenyl)ethan-1-amine (40.8 mg, 293 μmol).

LC-MS (method 1): Rₜ=102.00 min; MS (ESIpos): m/z=473 [M+H]⁺

¹H-NMR (400 MHz, DMSO-d6) δ [ppm]: 1.233 (0.50), 1.341 (11.77), 1.358 (11.82), 1.463 (0.44), 1.480 (0.41), 2.518 (7.97), 2.523 (4.73), 2.819 (3.16), 2.837 (5.23), 2.854 (3.39), 3.987 (2.42), 4.010 (15.90), 4.024 (5.90), 4.045 (2.23), 4.097 (3.56), 4.115 (5.61), 4.132 (3.38), 4.786 (1.60), 4.805 (2.13), 4.823 (1.52), 6.162 (9.18), 6.622 (16.00), 6.876 (3.82), 6.897 (3.67), 6.995 (3.46), 7.114 (4.68), 7.119 (1.60), 7.130 (2.00), 7.136 (9.47), 7.142 (1.96), 7.153 (1.78), 7.159 (5.41), 7.180 (7.08), 7.349 (4.66), 7.354 (2.16), 7.364 (7.47), 7.370 (4.78), 7.380 (1.74), 7.384 (3.81), 7.639 (5.15), 8.231 (8.28), 8.235 (7.89).

### Example 275

### 2′-[6-amino-5-(difluoromethoxy)pyridin-3-yl]-N-[(1R)-1-(2-cyanopyridin-4-yl)ethyl]-5′,6′-dihydrospiro[azetidine-3,4′-pyrrolo[1,2-b]pyrazole]-1-carboxamide

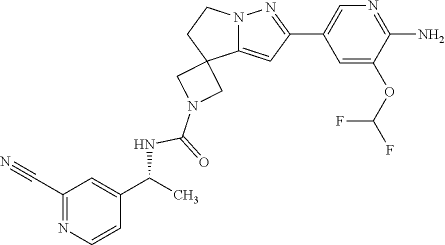 [SMILES: C[C@@H](NC(=O)N1CC2(CCn3nc(-c4cnc(N)c(OC(F)F)c4)cc32)C1)c1ccnc(C#N)c1]

Example 275 was prepared in analogy to Example 31 using 3-(difluoromethoxy)-5-(5′,6′-dihydrospiro[azetidine-3,4′-pyrrolo[1,2-b]pyrazol]-2′-yl)pyridin-2-amine (75.0 mg, 244 μmol) and 4-[(1R)-1-aminoethyl]pyridine-2-carbonitrile (43.1 mg, 293 μmol).

LC-MS (method 1): Rₜ=0.88 min; MS (ESIpos): m/z=481 [M+H]⁺

¹H-NMR (400 MHz, DMSO-d6) δ [ppm]: 1.044 (0.55), 1.367 (11.57), 1.385 (11.84), 1.402 (0.43), 2.073 (0.67), 2.518 (0.93), 2.523 (0.57), 2.841 (2.94), 2.858 (4.78), 2.876 (3.17), 4.022 (2.90), 4.033 (1.72), 4.043 (4.83), 4.053 (5.82), 4.060 (5.90), 4.081 (1.86), 4.089 (4.35), 4.109 (4.74), 4.124 (5.53), 4.142 (3.24), 4.833 (1.64), 4.851 (2.44), 4.870 (1.63), 6.148 (1.05), 6.170 (8.70), 6.630 (0.48), 6.641 (16.00), 6.999 (3.64), 7.052 (3.62), 7.071 (3.48), 7.183 (7.57), 7.202 (0.70), 7.367 (3.21), 7.559 (0.40), 7.642 (4.77), 7.644 (4.89), 7.646 (4.79), 7.684 (2.96), 7.687 (3.07), 7.697 (3.14), 7.700 (3.08), 8.000 (5.53), 8.002 (5.51), 8.004 (5.46), 8.080 (0.67), 8.085 (0.64), 8.238 (8.33), 8.243 (8.12), 8.694 (5.24), 8.707 (4.90).

### Example 276

### (rac)-2′-[6-amino-5-(trifluoromethoxy)pyridin-3-yl]-N-[1-(2-fluorophenyl)cyclobutyl]-5′,6′-dihydrospiro[pyrrolidine-3,4′-pyrrolo[1,2-b]pyrazole]-1-carboxamide

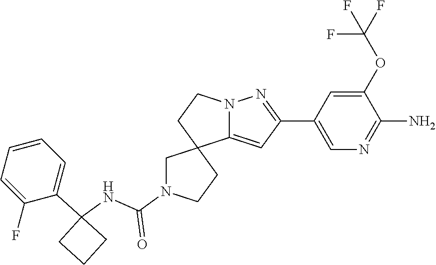 [SMILES: Nc1ncc(-c2cc3n(n2)CCC32CCN(C(=O)NC3(c4ccccc4F)CCC3)C2)cc1OC(F)(F)F]

Example 276 was prepared in analogy to Example 31 using (rac)-5-[5′,6′-dihydrospiro[pyrrolidine-3,4′-pyrrolo[1,2-b]pyrazol]-2′-yl]-3-(trifluoromethoxy)pyridin-2-amine (75.0 mg, 221 μmol) and 1-(2-fluorophenyl)cyclobutan-1-amine-hydrochloride salt (53.5 mg, 265 μmol).

LC-MS (method 1): Rₜ=1.20 min; MS (ESIpos): m/z=531 [M+H]⁺

¹H-NMR (400 MHz, DMSO-d6) δ [ppm]: 0.847 (0.56), 1.033 (0.92), 1.051 (1.69), 1.064 (0.50), 1.068 (1.06), 1.227 (3.09), 1.701 (0.59), 1.714 (0.82), 1.723 (1.27), 1.729 (1.32), 1.736 (1.39), 1.743 (1.27), 1.750 (1.48), 1.764 (1.01), 2.003 (2.63), 2.022 (3.69), 2.031 (4.75), 2.050 (3.10), 2.064 (1.42), 2.091 (0.46), 2.331 (1.04), 2.459 (0.97), 2.518 (16.00), 2.522 (12.70), 3.271 (0.54), 3.298 (0.48), 3.305 (1.54), 3.313 (1.41), 3.323 (0.93), 3.331 (1.58), 3.338 (1.04), 3.345 (1.80), 3.352 (2.47), 3.361 (9.53), 3.414 (11.00), 3.417 (10.12), 3.438 (3.18), 3.449 (2.92), 3.457 (1.67), 3.466 (1.88), 3.478 (3.52), 3.488 (2.18), 3.506 (1.79), 3.522 (1.06), 3.531 (0.49), 3.535 (0.48), 4.141 (4.05), 4.158 (8.48), 4.176 (4.14), 5.750 (0.90), 6.335 (12.48), 6.501 (12.12), 6.587 (7.06), 7.025 (1.92), 7.028 (2.07), 7.046 (2.53), 7.048 (2.80), 7.055 (2.12), 7.057 (2.41), 7.063 (2.84), 7.067 (2.44), 7.077 (3.46), 7.082 (6.15), 7.086 (4.47), 7.101 (3.55), 7.104 (2.89), 7.183 (1.29), 7.188 (1.47), 7.196 (1.58), 7.201 (2.32), 7.207 (1.92), 7.215 (2.05), 7.221 (2.05), 7.226 (1.17), 7.234 (0.98), 7.239 (0.89), 7.404 (2.00), 7.408 (2.02), 7.425 (3.26), 7.444 (1.92), 7.448 (1.62), 7.721 (4.56), 7.725 (6.14), 7.729 (4.28), 8.290 (9.93), 8.296 (9.26).

### Example 277

### (rac)-2-[6-amino-5-(trifluoromethoxy)pyridin-3-yl]-N-[2-(2-chlorophenyl)propan-2-yl]-6,7-dihydrospiro[pyrazolo[5,1-c][1,4]oxazine-4,3′-pyrrolidine]-1′-carboxamide

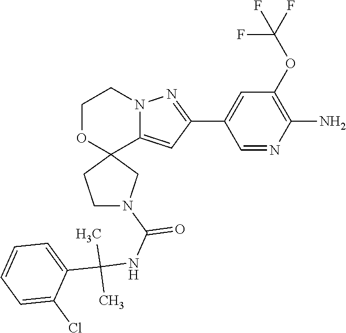 [SMILES: CC(C)(NC(=O)N1CCC2(C1)OCCn1nc(-c3cnc(N)c(OC(F)(F)F)c3)cc12)c1ccccc1Cl]

Example 277 was prepared in analogy to Example 31 using (rac)-5-[6,7-dihydrospiro[pyrazolo[5,1-c][1,4]oxazine-4,3′-pyrrolidin]-2-yl]-3-(trifluoromethoxy)pyridin-2-amine (75.0 mg, 211 μmol) and 2-(2-chlorophenyl)propan-2-amine-hydrochloride salt (52.2 mg, 253 μmol).

LC-MS (method 1): Rₜ=1.20 min; MS (ESIpos): m/z=551 [M+H]⁺

¹H-NMR (400 MHz, DMSO-d6) δ [ppm]: 1.238 (0.47), 1.254 (0.48), 1.678 (16.00), 1.701 (15.31), 1.765 (0.62), 2.171 (0.41), 2.202 (1.01), 2.228 (1.49), 2.251 (0.78), 2.291 (1.20), 2.306 (1.42), 2.323 (1.25), 2.327 (1.25), 2.539 (1.30), 2.669 (0.55), 2.728 (0.64), 2.888 (0.71), 3.391 (1.62), 3.416 (0.77), 3.443 (2.19), 3.471 (2.54), 3.537 (1.03), 3.558 (1.80), 3.580 (0.86), 3.744 (2.06), 3.773 (1.77), 4.049 (0.66), 4.064 (1.40), 4.081 (1.69), 4.101 (2.84), 4.113 (4.92), 4.126 (6.79), 6.152 (6.01), 6.559 (7.44), 6.692 (9.92), 7.166 (1.04), 7.170 (1.11), 7.184 (2.76), 7.188 (2.83), 7.203 (2.41), 7.207 (2.35), 7.234 (1.77), 7.238 (2.09), 7.256 (3.06), 7.272 (1.44), 7.275 (1.37), 7.295 (4.04), 7.299 (3.71), 7.315 (2.99), 7.318 (2.70), 7.454 (2.98), 7.458 (3.06), 7.474 (2.56), 7.478 (2.48), 7.745 (4.55), 8.325 (6.02), 8.330 (6.04).

### Example 278

### (rac)-2-[6-amino-5-(trifluoromethoxy)pyridin-3-yl]-N-[2-(4-fluorophenyl)propan-2-yl]-6,7-dihydrospiro[pyrazolo[5,1-c][1,4]oxazine-4,3′-pyrrolidine]-1′-carboxamide

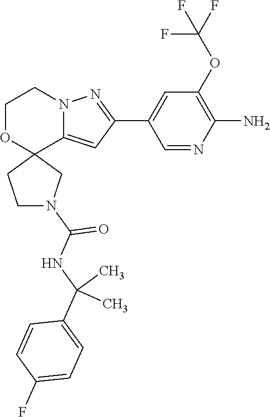 [SMILES: CC(C)(NC(=O)N1CCC2(C1)OCCn1nc(-c3cnc(N)c(OC(F)(F)F)c3)cc12)c1ccc(F)cc1]

Example 278 was prepared in analogy to Example 31 using (rac)-5-[6,7-dihydrospiro[pyrazolo[5,1-c][1,4]oxazine-4,3′-pyrrolidin]-2-yl]-3-(trifluoromethoxy)pyridin-2-amine (75.0 mg, 211 μmol) and 2-(4-fluorophenyl)propan-2-amine (38.8 mg, 253 μmol).

LC-MS (method 1): Rₜ=1.17 min; MS (ESIpos): m/z=535 [M+H]⁺

¹H-NMR (400 MHz, DMSO-d6) δ [ppm]: 0.833 (0.71), 0.852 (1.03), 1.232 (4.66), 1.295 (0.56), 1.333 (0.69), 1.348 (0.59), 1.549 (10.08), 1.554 (9.85), 2.074 (16.00), 2.210 (0.52), 2.235 (0.69), 2.322 (2.58), 2.327 (3.22), 2.332 (2.41), 2.518 (12.78), 2.523 (8.56), 2.539 (0.51), 2.665 (2.04), 2.669 (2.88), 2.673 (2.07), 3.159 (2.43), 3.171 (2.46), 3.369 (0.85), 3.392 (0.68), 3.470 (0.82), 3.504 (1.00), 3.570 (0.47), 3.591 (0.83), 3.774 (1.03), 3.803 (0.83), 4.079 (0.66), 4.096 (1.36), 4.111 (2.01), 4.123 (3.05), 4.135 (2.92), 5.758 (0.44), 6.099 (3.02), 6.556 (4.39), 6.715 (6.04), 7.046 (2.13), 7.068 (4.50), 7.091 (2.52), 7.351 (2.19), 7.365 (2.49), 7.373 (2.35), 7.387 (2.07), 7.745 (2.30), 8.325 (3.84), 8.330 (3.68).

### Example 279

### (rac)-2′-[6-amino-5-(trifluoromethoxy)pyridin-3-yl]-N-[2-(3-fluorophenyl)propan-2-yl]-5′,6′-dihydrospiro[pyrrolidine-3,4′-pyrrolo[1,2-b]pyrazole]-1-carboxamide

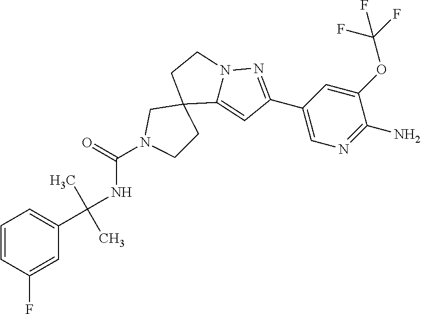 [SMILES: CC(C)(NC(=O)N1CCC2(CCn3nc(-c4cnc(N)c(OC(F)(F)F)c4)cc32)C1)c1cccc(F)c1]

Example 279 was prepared in analogy to Example 31 using (rac)-5-[5′,6′-dihydrospiro[pyrrolidine-3,4′-pyrrolo[1,2-b]pyrazol]-2′-yl]-3-(trifluoromethoxy)pyridin-2-amine (75.0 mg, 221 μmol) and 2-(3-fluorophenyl)propan-2-amine (40.6 mg, 265 μmol).

LC-MS (method 1): Rₜ=1.17 min; MS (ESIpos): m/z=519 [M+H]⁺

¹H-NMR (400 MHz, DMSO-d6) δ [ppm]: 1.036 (3.70), 1.054 (7.76), 1.071 (4.11), 1.230 (0.50), 1.555 (16.00), 2.041 (0.81), 2.056 (1.64), 2.073 (2.40), 2.083 (0.72), 2.091 (0.76), 2.518 (2.60), 2.522 (1.27), 2.535 (2.94), 2.553 (2.02), 3.161 (0.65), 3.173 (0.67), 3.406 (0.61), 3.424 (2.19), 3.437 (1.74), 3.442 (2.14), 3.449 (3.09), 3.453 (2.49), 3.464 (2.58), 3.490 (0.68), 3.537 (0.42), 3.555 (0.81), 3.570 (0.61), 3.578 (0.59), 4.171 (2.08), 4.189 (3.41), 4.206 (2.01), 4.349 (0.47), 4.361 (0.90), 4.373 (0.45), 6.121 (4.15), 6.399 (10.02), 6.513 (5.92), 6.925 (0.61), 6.927 (0.66), 6.931 (0.74), 6.934 (0.76), 6.947 (1.33), 6.953 (1.46), 6.967 (0.73), 6.969 (0.75), 6.974 (0.84), 6.975 (0.77), 7.108 (1.00), 7.114 (1.36), 7.119 (1.12), 7.136 (1.01), 7.141 (1.40), 7.147 (1.08), 7.176 (1.54), 7.196 (2.13), 7.258 (1.28), 7.274 (1.42), 7.278 (1.99), 7.293 (1.91), 7.297 (0.95), 7.314 (0.79), 7.744 (2.26), 7.748 (3.07), 7.752 (2.12), 8.328 (5.19), 8.333 (5.26).

### Example 280

### 2′-[6-amino-5-(trifluoromethoxy)pyridin-3-yl]-N-[(1S)-1-(3-chloropyridin-4-yl)ethyl]-5′,6′-dihydrospiro[pyrrolidine-3,4′-pyrrolo[1,2-b]pyrazole]-1-carboxamide (Mixture of Stereoisomers)

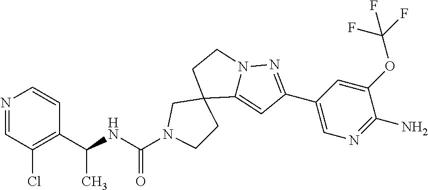 [SMILES: C[C@H](NC(=O)N1CCC2(CCn3nc(-c4cnc(N)c(OC(F)(F)F)c4)cc32)C1)c1ccncc1Cl]

Example 280 was prepared in analogy to Example 31 using (rac)-5-[5′,6′-dihydrospiro[pyrrolidine-3,4′-pyrrolo[1,2-b]pyrazol]-2′-yl]-3-(trifluoromethoxy)pyridin-2-amine (75.0 mg, 221 μmol) and (1S)-1-(3-chloropyridin-4-yl)ethan-1-amine-hydrochloride salt (51.2 mg, 265 μmol).

LC-MS (method 1): Rₜ=0.99 min; MS (ESIpos): m/z=522 [M+H]⁺

¹H-NMR (400 MHz, DMSO-d6) δ [ppm]: 1.274 (0.47), 1.291 (0.49), 1.332 (12.00), 1.335 (11.94), 1.349 (12.65), 1.353 (11.99), 2.074 (5.14), 2.083 (4.03), 2.518 (6.46), 2.523 (7.26), 2.540 (7.70), 2.557 (4.48), 3.318 (0.57), 3.368 (0.85), 3.386 (0.42), 3.433 (2.83), 3.459 (5.41), 3.487 (3.97), 3.495 (3.55), 3.512 (2.29), 3.534 (1.69), 3.549 (1.04), 3.582 (0.70), 3.600 (1.04), 4.172 (3.86), 4.188 (6.86), 4.203 (3.73), 5.037 (0.52), 5.055 (2.05), 5.069 (2.98), 5.073 (3.06), 5.087 (2.03), 5.104 (0.55), 6.421 (16.00), 6.511 (13.40), 6.764 (2.54), 6.780 (3.40), 6.796 (2.31), 7.456 (3.34), 7.469 (3.38), 7.529 (3.63), 7.541 (3.75), 7.756 (6.50), 7.761 (6.21), 8.330 (11.58), 8.335 (10.86), 8.438 (3.81), 8.450 (3.71), 8.477 (5.60), 8.490 (5.43), 8.525 (10.94), 8.528 (10.79), 8.571 (0.40).

### Example 281

### 2′-[6-amino-5-(difluoromethoxy)pyridin-3-yl]-N-[2-(4-fluorophenyl)propan-2-yl]-5′,6′-dihydrospiro[azetidine-3,4′-pyrrolo[1,2-b]pyrazole]-1-carboxamide

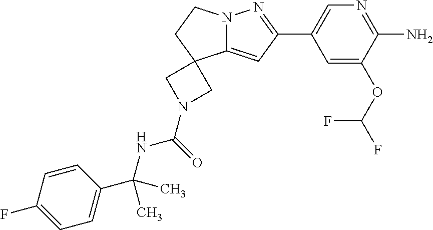 [SMILES: CC(C)(NC(=O)N1CC2(CCn3nc(-c4cnc(N)c(OC(F)F)c4)cc32)C1)c1ccc(F)cc1]

Example 281 was prepared in analogy to Example 31 using 3-(difluoromethoxy)-5-(5′,6′-dihydrospiro[azetidine-3,4′-pyrrolo[1,2-b]pyrazol]-2′-yl)pyridin-2-amine (75.0 mg, 244 μmol) and 2-(4-fluorophenyl)propan-2-amine (44.9 mg, 293 μmol).

LC-MS (method 1): Rₜ=1.09 min; MS (ESIpos): m/z=487 [M+H]⁺

¹H-NMR (400 MHz, DMSO-d6) δ [ppm]: 1.066 (1.49), 1.548 (16.00), 2.518 (1.40), 2.523 (0.94), 2.820 (1.28), 2.837 (2.08), 2.855 (1.36), 3.987 (0.52), 4.008 (8.06), 4.029 (0.51), 4.103 (1.41), 4.121 (2.21), 4.138 (1.34), 6.191 (2.01), 6.568 (3.09), 6.618 (6.86), 7.007 (1.48), 7.074 (1.91), 7.079 (0.61), 7.091 (0.74), 7.097 (4.03), 7.114 (0.64), 7.119 (2.11), 7.192 (3.07), 7.359 (2.04), 7.365 (0.89), 7.373 (2.52), 7.376 (2.46), 7.381 (2.10), 7.390 (0.73), 7.395 (1.82), 7.654 (1.99), 7.656 (2.03), 7.658 (2.02), 8.247 (3.54), 8.252 (3.56).

### Example 282

### 2′-[6-amino-5-(difluoromethoxy)pyridin-3-yl]-N-[2-(2-chlorophenyl)propan-2-yl]-5′,6′-dihydrospiro[azetidine-3,4′-pyrrolo[1,2-b]pyrazole]-1-carboxamide

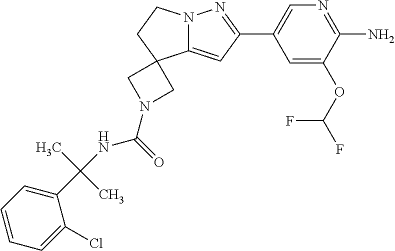 [SMILES: CC(C)(NC(=O)N1CC2(CCn3nc(-c4cnc(N)c(OC(F)F)c4)cc32)C1)c1ccccc1Cl]

Example 282 was prepared in analogy to Example 31 using 3-(difluoromethoxy)-5-(5′,6′-dihydrospiro[azetidine-3,4′-pyrrolo[1,2-b]pyrazol]-2′-yl)pyridin-2-amine (75.0 mg, 244 μmol) and 2-(2-chlorophenyl)propan-2-amine (49.7 mg, 293 μmol).

LC-MS (method 1): Rₜ=1.11 min; MS (ESIpos): m/z=503 [M+H]⁺

¹H-NMR (400 MHz, DMSO-d6) δ [ppm]: 1.066 (0.81), 1.156 (2.64), 1.634 (1.91), 1.677 (16.00), 2.074 (1.73), 2.518 (3.86), 2.523 (2.59), 2.728 (0.60), 2.801 (1.32), 2.819 (2.17), 2.836 (1.44), 2.888 (0.77), 3.993 (10.15), 4.095 (1.45), 4.112 (2.31), 4.130 (1.40), 6.167 (3.90), 6.541 (1.48), 6.567 (7.25), 6.646 (3.25), 7.005 (1.58), 7.190 (3.78), 7.204 (1.35), 7.208 (1.37), 7.223 (1.23), 7.227 (1.22), 7.252 (0.92), 7.256 (1.07), 7.272 (1.27), 7.276 (1.47), 7.290 (0.75), 7.294 (0.69), 7.332 (2.19), 7.336 (2.16), 7.351 (1.61), 7.355 (1.52), 7.374 (1.49), 7.459 (1.45), 7.463 (1.49), 7.479 (1.24), 7.483 (1.16), 7.646 (2.16), 8.241 (3.52), 8.245 (3.68).

### Example 283

### 2-[6-amino-5-(trifluoromethoxy)pyridin-3-yl]-N-[(1R)-1-(3-fluorophenyl)ethyl]-6,7-dihydrospiro[pyrazolo[5,1-c][1,4]oxazine-4,3′-pyrrolidine]-1′-carboxamide (Mixture of Stereoisomers)

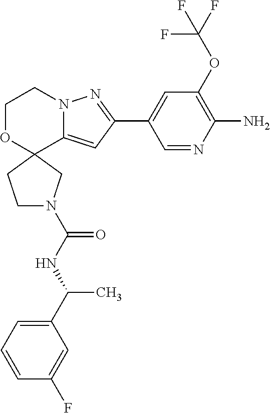 [SMILES: C[C@@H](NC(=O)N1CCC2(C1)OCCn1nc(-c3cnc(N)c(OC(F)(F)F)c3)cc12)c1cccc(F)c1]

Example 283 was prepared in analogy to Example 31 using (rac)-5-[6,7-dihydrospiro[pyrazolo[5,1-c][1,4]oxazine-4,3′-pyrrolidin]-2-yl]-3-(trifluoromethoxy)pyridin-2-amine (75.0 mg, 211 μmol) and (1R)-1-(3-fluorophenyl)ethan-1-amine (35.3 mg, 253 μmol).

LC-MS (method 1): Rₜ=1.10 min; MS (ESIpos): m/z=521 [M+H]⁺

¹H-NMR (400 MHz, DMSO-d6) δ [ppm]: 1.066 (0.58), 1.240 (0.65), 1.258 (1.00), 1.275 (0.56), 1.356 (15.96), 1.373 (16.00), 2.073 (0.51), 2.217 (1.34), 2.229 (1.15), 2.244 (1.58), 2.266 (0.79), 2.277 (0.64), 2.327 (1.96), 2.342 (1.91), 2.358 (1.19), 2.374 (1.02), 2.728 (1.09), 2.886 (1.31), 3.391 (2.00), 3.399 (2.79), 3.417 (1.73), 3.425 (1.56), 3.442 (0.87), 3.457 (1.79), 3.488 (2.97), 3.520 (2.11), 3.560 (1.00), 3.582 (1.75), 3.606 (1.64), 3.630 (1.77), 3.652 (0.78), 3.777 (2.03), 3.807 (1.90), 3.817 (2.12), 3.846 (1.70), 4.043 (0.74), 4.059 (1.32), 4.074 (2.16), 4.092 (2.55), 4.105 (3.89), 4.119 (4.92), 4.131 (9.29), 4.830 (1.67), 4.848 (2.33), 4.862 (1.64), 4.879 (0.48), 6.530 (2.66), 6.550 (5.67), 6.566 (8.45), 6.708 (7.29), 6.714 (8.27), 6.989 (1.52), 6.992 (1.57), 6.995 (1.68), 7.015 (3.21), 7.034 (1.75), 7.038 (1.76), 7.150 (3.41), 7.161 (4.67), 7.180 (8.14), 7.307 (1.40), 7.312 (1.52), 7.322 (1.73), 7.327 (3.44), 7.332 (2.45), 7.342 (2.37), 7.347 (3.08), 7.363 (1.05), 7.368 (0.88), 7.747 (5.37), 8.317 (5.29), 8.324 (7.27), 8.329 (5.48).

### Example 284

### 2′-[6-amino-5-(trifluoromethoxy)pyridin-3-yl]-N-(1-phenylcyclobutyl)-5′,6′-dihydrospiro[azetidine-3,4′-pyrrolo[1,2-b]pyrazole]-1-carboxamide

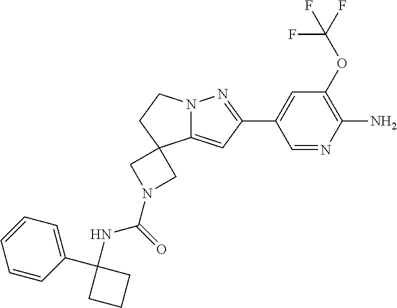 [SMILES: Nc1ncc(-c2cc3n(n2)CCC32CN(C(=O)NC3(c4ccccc4)CCC3)C2)cc1OC(F)(F)F]

Example 284 was prepared in analogy to Example 31 using 5-(5′,6′-dihydrospiro[azetidine-3,4′-pyrrolo[1,2-b]pyrazol]-2′-yl)-3-(trifluoromethoxy)pyridin-2-amine-hydrochloride salt (75.0 mg, 207 μmol) and 1-phenylcyclobutan-1-amine-hydrochloride salt (45.7 mg, 249 μmol).

LC-MS (method 1): Rₜ=1.15 min; MS (ESIneg): m/z=497 [M−H]⁻

¹H-NMR (400 MHz, DMSO-d6) δ [ppm]: 1.156 (0.50), 1.242 (0.90), 1.246 (0.65), 1.258 (1.47), 1.276 (0.80), 1.747 (0.44), 1.752 (0.47), 1.757 (0.41), 1.768 (0.94), 1.774 (0.97), 1.780 (0.68), 1.785 (0.70), 1.791 (0.99), 1.796 (1.15), 1.812 (0.68), 1.818 (0.56), 1.976 (0.46), 1.981 (0.59), 1.998 (1.10), 2.003 (0.82), 2.014 (0.72), 2.020 (0.98), 2.025 (0.96), 2.036 (0.51), 2.041 (0.51), 2.048 (0.50), 2.074 (0.98), 2.084 (11.36), 2.365 (0.91), 2.387 (1.57), 2.395 (2.15), 2.411 (2.61), 2.416 (2.23), 2.432 (1.62), 2.449 (1.82), 2.466 (2.29), 2.472 (2.77), 2.479 (2.01), 2.518 (3.38), 2.523 (2.04), 2.729 (1.80), 2.812 (2.81), 2.829 (4.60), 2.846 (3.00), 2.888 (2.25), 3.967 (1.96), 3.988 (11.55), 3.993 (11.40), 4.014 (1.94), 4.098 (3.12), 4.116 (4.94), 4.133 (3.03), 6.504 (7.58), 6.542 (0.69), 6.564 (0.41), 6.651 (16.00), 6.718 (0.57), 7.073 (6.79), 7.163 (0.81), 7.166 (1.53), 7.169 (0.92), 7.180 (1.14), 7.184 (3.91), 7.188 (1.38), 7.199 (1.50), 7.202 (2.72), 7.206 (1.50), 7.295 (4.28), 7.298 (1.77), 7.314 (7.20), 7.328 (1.45), 7.333 (4.40), 7.415 (6.53), 7.418 (7.37), 7.431 (1.61), 7.435 (5.57), 7.438 (4.23), 7.770 (3.23), 7.775 (4.58), 7.778 (3.12), 8.366 (7.44), 8.370 (7.65).

### Example 285

### 2′-[6-amino-5-(trifluoromethoxy)pyridin-3-yl]-N-benzyl-5′,6′-dihydrospiro[azetidine-3,4′-pyrrolo[1,2-b]pyrazole]-1-carboxamide

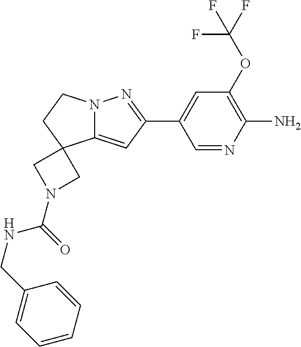 [SMILES: Nc1ncc(-c2cc3n(n2)CCC32CN(C(=O)NCc3ccccc3)C2)cc1OC(F)(F)F]

Example 285 was prepared in analogy to Example 31 using 5-(5′,6′-dihydrospiro[azetidine-3,4′-pyrrolo[1,2-b]pyrazol]-2′-yl)-3-(trifluoromethoxy)pyridin-2-amine-hydrochloride salt (75.0 mg, 207 μmol) and 1-phenylmethanamine (26.7 mg, 249 μmol).

LC-MS (method 1): Rₜ=1.03 min; MS (ESIpos): m/z=459 [M+H]⁺

¹H-NMR (400 MHz, DMSO-d6) δ [ppm]: 1.040 (0.88), 1.057 (1.11), 1.067 (1.22), 1.156 (2.22), 2.074 (1.60), 2.084 (0.90), 2.518 (1.82), 2.523 (1.24), 2.729 (6.30), 2.833 (2.79), 2.850 (4.52), 2.867 (2.99), 2.887 (7.60), 4.014 (1.58), 4.034 (14.65), 4.037 (14.32), 4.058 (1.55), 4.105 (3.06), 4.124 (4.76), 4.140 (2.93), 4.223 (12.89), 4.238 (13.13), 6.422 (1.12), 6.436 (2.12), 6.452 (1.11), 6.505 (8.06), 6.545 (0.70), 6.682 (16.00), 7.062 (1.55), 7.078 (3.38), 7.093 (1.54), 7.196 (0.57), 7.200 (1.19), 7.204 (0.94), 7.210 (1.35), 7.218 (3.48), 7.227 (3.42), 7.239 (6.61), 7.244 (3.44), 7.249 (2.42), 7.256 (7.69), 7.260 (5.32), 7.271 (3.46), 7.288 (9.73), 7.292 (12.67), 7.297 (2.95), 7.307 (11.09), 7.310 (7.58), 7.312 (6.52), 7.324 (7.03), 7.328 (4.78), 7.339 (0.94), 7.343 (2.13), 7.345 (1.31), 7.771 (3.05), 7.775 (4.33), 7.779 (2.93), 7.951 (0.92), 8.366 (7.23), 8.371 (7.43).

### Example 286

### 2′-[6-amino-5-(trifluoromethoxy)pyridin-3-yl]-N-[(1R)-1-(4-cyanopyridin-2-yl)ethyl]-5′,6′-dihydrospiro[azetidine-3,4′-pyrrolo[1,2-b]pyrazole]-1-carboxamide

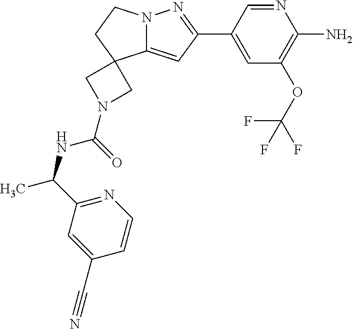 [SMILES: C[C@@H](NC(=O)N1CC2(CCn3nc(-c4cnc(N)c(OC(F)(F)F)c4)cc32)C1)c1cc(C#N)ccn1]

Example 286 was prepared in analogy to Example 31 using 5-(5′,6′-dihydrospiro[azetidine-3,4′-pyrrolo[1,2-b]pyrazol]-2′-yl)-3-(trifluoromethoxy)pyridin-2-amine-hydrochloride salt (75.0 mg, 207 μmol) and 2-[(1R)-1-aminoethyl]pyridine-4-carbonitrile (36.6 mg, 249 μmol).

LC-MS (method 1): Rₜ=0.92 min; MS (ESIpos): m/z=499 [M+H]⁺

¹H-NMR (400 MHz, DMSO-d6) δ [ppm]: 1.066 (2.05), 1.397 (11.00), 1.415 (11.00), 2.083 (1.88), 2.460 (0.45), 2.465 (0.55), 2.518 (1.84), 2.523 (1.21), 2.729 (0.51), 2.823 (2.88), 2.830 (3.15), 2.848 (4.77), 2.865 (3.20), 2.887 (0.70), 3.908 (1.96), 3.911 (1.96), 4.003 (2.95), 4.024 (4.83), 4.041 (8.08), 4.063 (1.49), 4.070 (4.45), 4.091 (2.99), 4.104 (3.53), 4.122 (5.57), 4.130 (1.82), 4.138 (3.38), 4.149 (0.83), 4.882 (1.65), 4.900 (2.38), 4.919 (1.61), 6.484 (4.28), 6.488 (2.41), 6.504 (8.96), 6.677 (16.00), 6.997 (3.72), 7.017 (3.57), 7.754 (0.79), 7.765 (3.76), 7.770 (4.88), 7.773 (3.30), 8.252 (3.42), 8.257 (6.27), 8.262 (3.38), 8.337 (1.52), 8.342 (1.46), 8.361 (7.99), 8.366 (7.83), 8.848 (6.60), 8.854 (6.39), 8.901 (7.37), 8.906 (7.37).

### Example 287

### 2′-[6-amino-5-(trifluoromethoxy)pyridin-3-yl]-N-[(1R)-1-(2-cyanopyridin-4-yl)ethyl]-5′,6′-dihydrospiro[azetidine-3,4′-pyrrolo[1,2-b]pyrazole]-1-carboxamide

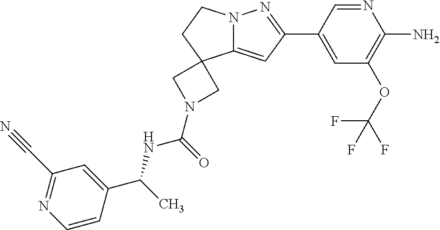 [SMILES: C[C@@H](NC(=O)N1CC2(CCn3nc(-c4cnc(N)c(OC(F)(F)F)c4)cc32)C1)c1ccnc(C#N)c1]

Example 287 was prepared in analogy to Example 31 using 5-(5′,6′-dihydrospiro[azetidine-3,4′-pyrrolo[1,2-b]pyrazol]-2′-yl)-3-(trifluoromethoxy)pyridin-2-amine-hydrochloride salt (75.0 mg, 207 μmol) and 4-[(1R)-1-aminoethyl]pyridine-2-carbonitrile (36.6 mg, 249 μmol).

LC-MS (method 1): Rₜ=0.94 min; MS (ESIneg): m/z=497 [M−H]⁻

¹H-NMR (400 MHz, DMSO-d6) δ [ppm]: 1.240 (1.04), 1.255 (1.17), 1.270 (0.73), 1.366 (10.98), 1.383 (11.13), 2.074 (2.53), 2.083 (1.46), 2.518 (3.40), 2.523 (2.40), 2.539 (9.58), 2.727 (0.95), 2.840 (2.81), 2.857 (4.54), 2.875 (3.04), 2.888 (1.29), 4.018 (2.82), 4.031 (1.54), 4.038 (4.54), 4.051 (5.99), 4.057 (6.16), 4.078 (1.44), 4.086 (4.10), 4.109 (4.58), 4.128 (5.22), 4.145 (3.08), 4.832 (1.57), 4.850 (2.35), 4.869 (1.57), 6.509 (6.94), 6.683 (16.00), 7.048 (3.57), 7.067 (3.43), 7.683 (2.91), 7.686 (2.95), 7.696 (3.11), 7.700 (3.02), 7.768 (3.33), 7.772 (4.72), 7.777 (3.20), 7.998 (5.21), 8.001 (5.26), 8.003 (5.42), 8.364 (8.08), 8.369 (7.96), 8.694 (5.12), 8.696 (5.14), 8.707 (4.88), 8.709 (4.88).

### Example 288

### 2′-[6-amino-5-(trifluoromethoxy)pyridin-3-yl]-N-[(1R)-1-(5-fluoropyridin-3-yl)ethyl]-5′,6′-dihydrospiro[pyrrolidine-3,4′-pyrrolo[1,2-b]pyrazole]-1-carboxamide (Mixture of Stereoisomers)

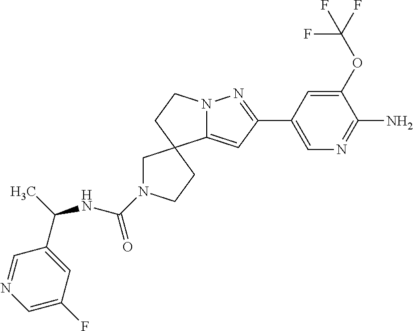 [SMILES: C[C@@H](NC(=O)N1CCC2(CCn3nc(-c4cnc(N)c(OC(F)(F)F)c4)cc32)C1)c1cncc(F)c1]

Example 288 was prepared in analogy to Example 31 using (rac)-5-[5′,6′-dihydrospiro[pyrrolidine-3,4′-pyrrolo[1,2-b]pyrazol]-2′-yl]-3-(trifluoromethoxy)pyridin-2-amine (75.0 mg, 221 μmol) and (1R)-1-(5-fluoropyridin-3-yl)ethan-1-amine (37.2 mg, 265 μmol).

LC-MS (method 1): Rₜ=0.95 min; MS (ESIneg): m/z=504 [M−H]⁻

¹H-NMR (400 MHz, DMSO-d6) δ [ppm]: 1.067 (7.42), 1.154 (0.90), 1.395 (12.36), 1.401 (13.25), 1.413 (12.84), 1.418 (12.87), 2.050 (2.89), 2.062 (4.44), 2.069 (4.61), 2.074 (4.42), 2.077 (4.20), 2.108 (0.68), 2.518 (7.56), 2.536 (9.89), 2.553 (6.30), 2.727 (0.40), 2.886 (0.49), 3.406 (0.75), 3.423 (3.74), 3.431 (2.94), 3.448 (7.92), 3.456 (7.12), 3.466 (6.87), 3.481 (4.40), 3.491 (2.40), 3.507 (2.60), 3.527 (1.93), 3.534 (1.08), 3.541 (1.30), 3.552 (1.16), 3.560 (1.00), 3.567 (0.92), 3.578 (1.53), 3.586 (1.03), 3.593 (1.10), 3.604 (1.06), 3.619 (0.58), 3.943 (1.24), 4.164 (5.85), 4.181 (9.82), 4.198 (5.67), 4.889 (0.53), 4.901 (1.92), 4.907 (2.05), 4.920 (2.99), 4.924 (2.97), 4.939 (2.08), 4.956 (0.54), 5.779 (0.95), 6.399 (6.74), 6.411 (16.00), 6.504 (13.68), 6.590 (3.08), 6.604 (3.73), 6.609 (3.64), 6.623 (3.02), 7.641 (1.59), 7.647 (2.14), 7.652 (1.68), 7.667 (1.66), 7.672 (2.28), 7.677 (3.14), 7.682 (2.27), 7.687 (1.69), 7.703 (1.58), 7.708 (2.07), 7.713 (1.60), 7.754 (8.52), 8.325 (13.55), 8.330 (13.14), 8.398 (6.79), 8.405 (6.86), 8.411 (6.88), 8.418 (6.55), 8.433 (3.45), 8.437 (6.29), 8.442 (3.57), 8.452 (3.35), 8.457 (5.95), 8.461 (3.29).

### Example 289

### (rac)-2′-[6-amino-5-(trifluoromethoxy)pyridin-3-yl]-N-[2-(pyridin-4-yl)propan-2-yl]-5′,6′-dihydrospiro[pyrrolidine-3,4′-pyrrolo[1,2-b]pyrazole]-1-carboxamide

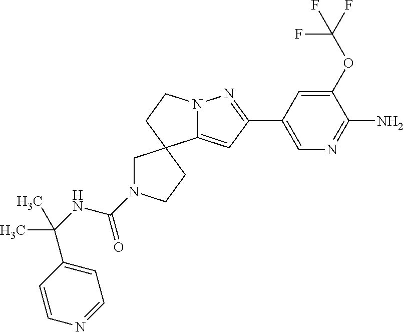 [SMILES: CC(C)(NC(=O)N1CCC2(CCn3nc(-c4cnc(N)c(OC(F)(F)F)c4)cc32)C1)c1ccncc1]

Example 289 was prepared in analogy to Example 31 using (rac)-5-[5′,6′-dihydrospiro[pyrrolidine-3,4′-pyrrolo[1,2-b]pyrazol]-2′-yl]-3-(trifluoromethoxy)pyridin-2-amine (75.0 mg, 221 μmol) and 2-(pyridin-4-yl)propan-2-amine (36.1 mg, 265 μmol).

LC-MS (method 1): Rₜ=0.95 min; MS (ESIneg): m/z=500 [M−H]⁻

¹H-NMR (400 MHz, DMSO-d6) δ [ppm]: 1.066 (0.71), 1.486 (3.82), 1.537 (16.00), 1.907 (0.60), 2.043 (0.87), 2.059 (1.69), 2.076 (1.87), 2.083 (0.92), 2.095 (0.79), 2.107 (0.43), 2.518 (3.97), 2.536 (2.89), 2.539 (3.12), 2.555 (1.92), 2.729 (0.59), 2.888 (0.73), 3.403 (0.49), 3.423 (1.27), 3.449 (3.35), 3.459 (2.83), 3.485 (0.60), 3.539 (0.46), 3.557 (0.83), 3.572 (0.67), 4.172 (2.09), 4.189 (3.83), 4.207 (2.02), 6.221 (4.04), 6.423 (8.42), 6.517 (6.11), 7.310 (5.40), 7.315 (3.51), 7.322 (3.55), 7.326 (5.40), 7.502 (0.68), 7.506 (0.48), 7.513 (0.49), 7.518 (0.74), 7.753 (2.27), 7.758 (3.06), 8.336 (4.67), 8.341 (4.61), 8.414 (5.84), 8.419 (3.60), 8.426 (3.49), 8.430 (5.52), 8.558 (0.63), 8.562 (0.43), 8.570 (0.41), 8.573 (0.58).

### Example 290

### (rac)-2′-[6-amino-5-(trifluoromethoxy)pyridin-3-yl]-N-(1-phenylcyclobutyl)-5′,6′-dihydrospiro[pyrrolidine-3,4′-pyrrolo[1,2-b]pyrazole]-1-carboxamide

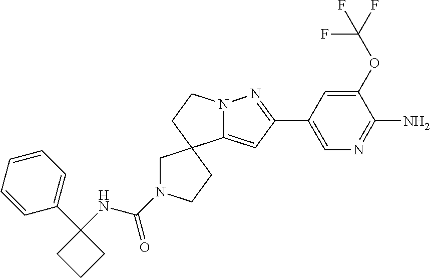 [SMILES: Nc1ncc(-c2cc3n(n2)CCC32CCN(C(=O)NC3(c4ccccc4)CCC3)C2)cc1OC(F)(F)F]

Example 290 was prepared in analogy to Example 31 using (rac)-5-[5′,6′-dihydrospiro[pyrrolidine-3,4′-pyrrolo[1,2-b]pyrazol]-2′-yl]-3-(trifluoromethoxy)pyridin-2-amine (75.0 mg, 221 μmol) and 1-phenylcyclobutan-1-amine-hydrochloride salt (48.7 mg, 265 μmol).

LC-MS (method 1): Rₜ=1.17 min; MS (ESIneg): m/z=511 [M−H]⁻

¹H-NMR (400 MHz, DMSO-d6) δ [ppm]: 1.000 (1.59), 1.016 (1.58), 1.043 (0.80), 1.066 (6.87), 1.156 (9.86), 1.239 (1.19), 1.254 (1.39), 1.271 (0.67), 1.746 (0.74), 1.763 (1.46), 1.768 (1.55), 1.785 (1.63), 1.790 (1.78), 1.807 (1.10), 1.829 (0.41), 1.964 (0.96), 1.979 (1.70), 2.002 (1.70), 2.007 (1.75), 2.030 (2.59), 2.043 (3.72), 2.059 (4.11), 2.075 (1.77), 2.088 (0.85), 2.356 (1.40), 2.385 (3.17), 2.401 (3.74), 2.423 (2.29), 2.449 (2.16), 2.470 (4.14), 2.518 (13.18), 2.523 (10.55), 2.539 (4.06), 2.555 (0.67), 2.728 (2.71), 2.888 (3.31), 3.367 (0.52), 3.375 (1.02), 3.396 (3.20), 3.421 (8.07), 3.433 (6.04), 3.459 (1.45), 3.504 (1.09), 3.521 (1.99), 3.537 (1.57), 3.546 (1.42), 3.563 (0.77), 3.939 (1.13), 4.159 (4.64), 4.177 (8.96), 4.194 (4.49), 6.371 (16.00), 6.511 (13.43), 6.541 (1.39), 6.696 (8.02), 7.131 (2.32), 7.149 (5.73), 7.167 (3.75), 7.258 (6.65), 7.278 (10.79), 7.297 (6.22), 7.383 (0.43), 7.417 (9.38), 7.419 (10.12), 7.437 (8.20), 7.440 (6.16), 7.733 (4.96), 7.738 (6.71), 7.741 (4.65), 7.942 (1.00), 7.951 (0.46), 8.312 (10.59), 8.316 (10.48).

### Example 291

### (rac)-2′-[6-amino-5-(trifluoromethoxy)pyridin-3-yl]-N-[1-(pyridin-4-yl)cyclobutyl]-5′,6′-dihydrospiro[pyrrolidine-3,4′-pyrrolo[1,2-b]pyrazole]-1-carboxamide

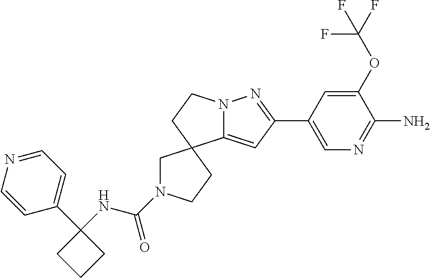 [SMILES: Nc1ncc(-c2cc3n(n2)CCC32CCN(C(=O)NC3(c4ccncc4)CCC3)C2)cc1OC(F)(F)F]

Example 291 was prepared in analogy to Example 31 using (rac)-5-[5′,6′-dihydrospiro[pyrrolidine-3,4′-pyrrolo[1,2-b]pyrazol]-2′-yl]-3-(trifluoromethoxy)pyridin-2-amine (75.0 mg, 221 μmol) and 1-(pyridin-4-yl)cyclobutan-1-amine (39.3 mg, 265 μmol).

LC-MS (method 1): Rₜ=0.97 min; MS (ESIneg): m/z=512 [M−H]⁻

¹H-NMR (400 MHz, DMSO-d6) δ [ppm]: 1.017 (0.84), 1.033 (0.85), 1.058 (0.48), 1.066 (2.98), 1.123 (0.48), 1.136 (1.35), 1.153 (1.46), 1.204 (0.42), 1.236 (1.78), 1.246 (4.77), 1.254 (3.26), 1.262 (5.19), 1.268 (4.93), 1.285 (4.48), 1.812 (0.84), 1.829 (1.60), 1.835 (1.74), 1.857 (2.04), 1.876 (1.32), 1.897 (0.52), 1.972 (0.42), 1.988 (0.84), 1.995 (1.09), 2.011 (2.04), 2.033 (2.41), 2.038 (2.38), 2.047 (2.96), 2.061 (4.32), 2.077 (4.42), 2.094 (1.79), 2.126 (0.48), 2.362 (1.47), 2.392 (3.60), 2.408 (4.16), 2.413 (4.08), 2.429 (4.25), 2.454 (3.74), 2.518 (10.29), 2.523 (6.57), 2.530 (6.62), 2.535 (6.58), 2.539 (6.75), 2.551 (4.04), 2.565 (1.47), 2.585 (0.76), 2.784 (0.52), 3.114 (0.96), 3.124 (1.00), 3.133 (1.09), 3.143 (1.08), 3.162 (0.75), 3.418 (6.35), 3.445 (12.32), 3.486 (2.46), 3.546 (2.45), 3.585 (1.49), 3.601 (1.02), 3.611 (0.93), 3.628 (0.76), 4.019 (1.85), 4.168 (4.96), 4.185 (9.53), 4.202 (4.76), 6.412 (16.00), 6.518 (9.77), 6.942 (7.52), 7.429 (0.42), 7.509 (9.82), 7.524 (9.42), 7.553 (0.68), 7.557 (0.46), 7.564 (0.44), 7.568 (0.66), 7.684 (0.61), 7.748 (5.44), 7.752 (7.39), 7.756 (5.24), 8.326 (11.59), 8.331 (11.50), 8.497 (0.49), 8.523 (11.55), 8.527 (7.63), 8.539 (11.12), 8.673 (0.67), 8.677 (0.47), 8.688 (0.60).

### Example 292

### 2′-[6-amino-5-(difluoromethoxy)pyridin-3-yl]-N-[2-(3-fluorophenyl)propan-2-yl]-5′,6′-dihydrospiro[azetidine-3,4′-pyrrolo[1,2-b]pyrazole]-1-carboxamide

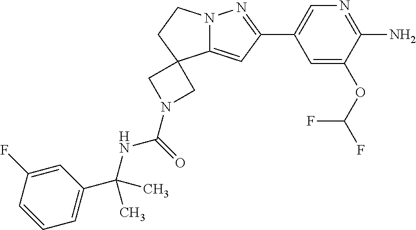 [SMILES: CC(C)(NC(=O)N1CC2(CCn3nc(-c4cnc(N)c(OC(F)F)c4)cc32)C1)c1cccc(F)c1]

Example 292 was prepared in analogy to Example 31 using 3-(difluoromethoxy)-5-(5′,6′-dihydrospiro[azetidine-3,4′-pyrrolo[1,2-b]pyrazol]-2′-yl)pyridin-2-amine (75.0 mg, 244 μmol) and 2-(3-fluorophenyl)propan-2-amine (44.9 mg, 293 μmol).

LC-MS (method 1): Rₜ=1.08 min; MS (ESIpos): m/z=487 [M+H]⁺

¹H-NMR (400 MHz, DMSO-d6) δ [ppm]: 1.065 (12.56), 1.155 (0.52), 1.239 (1.75), 1.247 (5.98), 1.258 (4.08), 1.263 (6.52), 1.271 (6.16), 1.276 (2.65), 1.288 (5.71), 1.547 (16.00), 1.624 (0.91), 2.518 (4.58), 2.523 (3.29), 2.827 (1.26), 2.844 (2.08), 2.862 (1.36), 3.112 (0.78), 3.123 (0.82), 3.131 (0.81), 3.141 (0.79), 3.583 (0.49), 3.594 (0.50), 3.600 (0.65), 3.610 (0.65), 3.616 (0.50), 3.627 (0.47), 3.999 (0.75), 4.020 (5.91), 4.024 (5.76), 4.046 (0.82), 4.106 (1.40), 4.124 (2.23), 4.141 (1.40), 6.263 (0.83), 6.622 (7.38), 6.965 (0.43), 6.972 (0.49), 6.985 (0.88), 6.992 (1.00), 7.008 (0.56), 7.016 (1.46), 7.036 (0.99), 7.112 (0.70), 7.118 (0.93), 7.122 (0.76), 7.140 (0.69), 7.144 (0.96), 7.151 (0.78), 7.163 (1.16), 7.185 (1.10), 7.201 (3.17), 7.290 (0.95), 7.304 (0.81), 7.320 (0.92), 7.324 (1.27), 7.339 (1.21), 7.344 (0.71), 7.360 (0.55), 7.385 (1.15), 7.671 (1.94), 8.244 (3.62), 8.248 (3.46).

### Example 293

### 2′-[6-amino-5-(difluoromethoxy)pyridin-3-yl]-N-[(1S)-1-(3-chloropyridin-4-yl)ethyl]-5′,6′-dihydrospiro[azetidine-3,4′-pyrrolo[1,2-b]pyrazole]-1-carboxamide

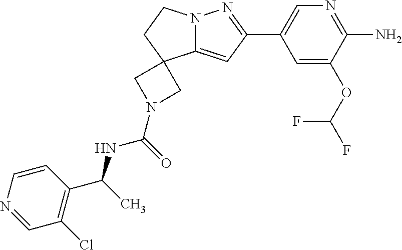 [SMILES: C[C@H](NC(=O)N1CC2(CCn3nc(-c4cnc(N)c(OC(F)F)c4)cc32)C1)c1ccncc1Cl]

Example 293 was prepared in analogy to Example 31 using 3-(difluoromethoxy)-5-(5′,6′-dihydrospiro[azetidine-3,4′-pyrrolo[1,2-b]pyrazol]-2′-yl)pyridin-2-amine (75.0 mg, 244 μmol) and (1S)-1-(3-chloropyridin-4-yl)ethan-1-amine-hydrochloride salt (56.5 mg, 293 μmol).

LC-MS (method 1): Rₜ=0.90 min; MS (ESIneg): m/z=488 [M−H]⁻

¹H-NMR (400 MHz, DMSO-d6) δ [ppm]: 1.212 (0.40), 1.231 (1.83), 1.263 (1.15), 1.281 (1.19), 1.328 (13.28), 1.346 (13.41), 2.518 (7.29), 2.522 (5.21), 2.838 (3.18), 2.855 (5.34), 2.872 (3.43), 3.824 (0.68), 4.022 (3.02), 4.047 (7.49), 4.054 (6.92), 4.081 (4.88), 4.106 (4.37), 4.124 (5.83), 4.141 (3.56), 5.005 (0.44), 5.023 (2.00), 5.042 (3.05), 5.059 (2.01), 5.078 (0.47), 6.171 (9.51), 6.431 (0.88), 6.635 (16.00), 7.000 (3.61), 7.184 (9.11), 7.200 (3.83), 7.369 (3.16), 7.483 (5.70), 7.496 (5.89), 7.643 (5.20), 8.213 (0.54), 8.218 (0.54), 8.238 (8.54), 8.243 (7.96), 8.418 (0.40), 8.517 (7.88), 8.530 (7.19), 8.548 (13.03).

### Example 294

### 2′-[6-amino-5-(difluoromethoxy)pyridin-3-yl]-N-[2-(3-chloropyridin-4-yl)propan-2-yl]-5′,6′-dihydrospiro[azetidine-3,4′-pyrrolo[1,2-b]pyrazole]-1-carboxamide

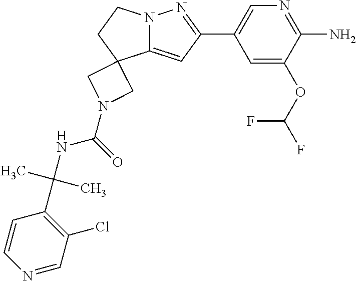 [SMILES: CC(C)(NC(=O)N1CC2(CCn3nc(-c4cnc(N)c(OC(F)F)c4)cc32)C1)c1ccncc1Cl]

Example 294 was prepared in analogy to Example 31 using 3-(difluoromethoxy)-5-(5′,6′-dihydrospiro[azetidine-3,4′-pyrrolo[1,2-b]pyrazol]-2′-yl)pyridin-2-amine (75.0 mg, 244 μmol) and 2-(3-chloropyridin-4-yl)propan-2-amine (50.0 mg, 293 μmol).

LC-MS (method 1): Rₜ=0.93 min; MS (ESIpos): m/z=504 [M+H]⁺

¹H-NMR (400 MHz, DMSO-d6) δ [ppm]: 1.066 (2.58), 1.232 (1.82), 1.498 (0.53), 1.560 (2.88), 1.645 (16.00), 2.518 (6.33), 2.523 (4.35), 2.728 (8.84), 2.808 (1.30), 2.825 (2.10), 2.842 (1.39), 2.888 (10.66), 3.980 (0.47), 4.000 (9.00), 4.096 (1.42), 4.114 (2.24), 4.130 (1.39), 6.169 (3.79), 6.577 (7.72), 6.857 (3.23), 7.006 (1.62), 7.190 (3.45), 7.374 (1.42), 7.433 (2.36), 7.446 (2.37), 7.647 (2.08), 7.951 (1.29), 8.243 (3.83), 8.248 (3.53), 8.316 (0.57), 8.330 (0.53), 8.417 (3.94), 8.425 (1.17), 8.430 (3.51), 8.469 (7.07).

### Example 295

### 2′-[6-amino-5-(difluoromethoxy)pyridin-3-yl]-N-[(1R)-1-(2-chloro-4-cyanophenyl)ethyl]-5′,6′-dihydrospiro[azetidine-3,4′-pyrrolo[1,2-b]pyrazole]-1-carboxamide

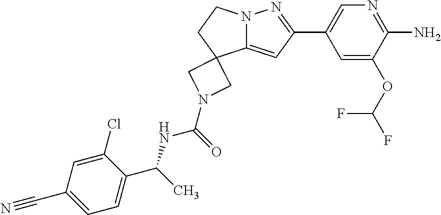 [SMILES: C[C@@H](NC(=O)N1CC2(CCn3nc(-c4cnc(N)c(OC(F)F)c4)cc32)C1)c1ccc(C#N)cc1Cl]

Example 295 was prepared in analogy to Example 31 using 3-(difluoromethoxy)-5-(5′,6′-dihydrospiro[azetidine-3,4′-pyrrolo[1,2-b]pyrazol]-2′-yl)pyridin-2-amine (75.0 mg, 244 μmol) and 4-[(1R)-1-aminoethyl]-3-chlorobenzonitrile (52.9 mg, 293 μmol).

LC-MS (method 1): Rₜ=1.04 min; MS (ESIneg): m/z=512 [M−H]⁻

¹H-NMR (400 MHz, DMSO-d6) δ [ppm]: 1.066 (2.20), 1.259 (0.98), 1.277 (0.91), 1.321 (15.42), 1.339 (15.48), 2.074 (0.68), 2.539 (1.98), 2.834 (4.36), 2.852 (7.61), 2.869 (4.62), 3.814 (0.75), 4.015 (4.90), 4.036 (12.64), 4.045 (9.39), 4.074 (6.45), 4.095 (4.18), 4.106 (5.20), 4.123 (8.06), 4.140 (4.72), 5.078 (0.60), 5.096 (2.60), 5.114 (3.91), 5.132 (2.59), 5.150 (0.62), 6.228 (5.08), 6.433 (0.70), 6.639 (16.00), 7.006 (3.69), 7.190 (9.81), 7.205 (5.02), 7.374 (3.46), 7.625 (0.44), 7.658 (13.85), 7.678 (7.67), 7.857 (5.01), 7.861 (5.26), 7.877 (4.08), 7.881 (4.35), 7.978 (0.43), 8.005 (9.38), 8.009 (9.26), 8.214 (0.56), 8.237 (9.23), 8.241 (9.45).

### Example 296

### 2′-[6-amino-5-(difluoromethoxy)pyridin-3-yl]-N-[1-(2-fluorophenyl)cyclobutyl]-5′,6′-dihydrospiro[azetidine-3,4′-pyrrolo[1,2-b]pyrazole]-1-carboxamide

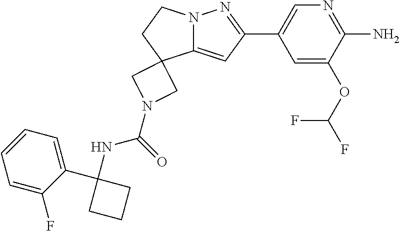 [SMILES: Nc1ncc(-c2cc3n(n2)CCC32CN(C(=O)NC3(c4ccccc4F)CCC3)C2)cc1OC(F)F]

Example 296 was prepared in analogy to Example 31 using 3-(difluoromethoxy)-5-(5′,6′-dihydrospiro[azetidine-3,4′-pyrrolo[1,2-b]pyrazol]-2′-yl)pyridin-2-amine (75.0 mg, 244 μmol) and 1-(2-fluorophenyl)cyclobutan-1-amine-hydrochloride salt (59.1 mg, 293 μmol).

LC-MS (method 1): Rₜ=1.11 min; MS (ESIpos): m/z=499 [M+H]⁺

¹H-NMR (400 MHz, DMSO-d6) δ [ppm]: 1.714 (0.44), 1.720 (0.51), 1.726 (0.62), 1.734 (0.95), 1.740 (1.00), 1.748 (1.04), 1.755 (0.92), 1.761 (1.14), 1.775 (0.69), 1.782 (0.48), 2.019 (0.75), 2.039 (1.27), 2.061 (1.16), 2.066 (1.17), 2.074 (3.69), 2.087 (0.70), 2.458 (0.71), 2.518 (4.76), 2.523 (4.99), 2.540 (2.12), 2.571 (0.45), 2.786 (2.95), 2.803 (4.89), 2.821 (3.14), 3.936 (2.08), 3.956 (12.51), 3.962 (12.24), 3.983 (2.03), 4.081 (3.25), 4.098 (5.13), 4.116 (3.11), 6.159 (8.56), 6.575 (16.00), 6.994 (3.53), 7.051 (6.99), 7.065 (1.66), 7.068 (1.76), 7.086 (2.02), 7.089 (2.55), 7.091 (2.43), 7.094 (3.83), 7.110 (3.88), 7.113 (4.24), 7.129 (2.82), 7.132 (1.99), 7.179 (7.27), 7.219 (0.96), 7.223 (1.11), 7.232 (1.15), 7.237 (1.72), 7.244 (1.45), 7.250 (1.08), 7.256 (1.60), 7.262 (0.76), 7.271 (0.70), 7.275 (0.65), 7.363 (3.00), 7.411 (1.49), 7.415 (1.51), 7.431 (2.52), 7.435 (2.51), 7.451 (1.45), 7.456 (1.16), 7.634 (4.80), 7.636 (4.70), 8.222 (8.17), 8.227 (7.57).

### Example 297

### (rac)-2′-[6-amino-5-(trifluoromethoxy)pyridin-3-yl]-N-[2-(2-chlorophenyl)propan-2-yl]-5′,6′-dihydrospiro[pyrrolidine-3,4′-pyrrolo[1,2-b]pyrazole]-1-carboxamide

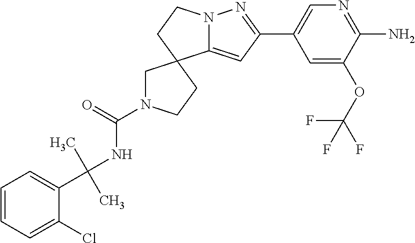 [SMILES: CC(C)(NC(=O)N1CCC2(CCn3nc(-c4cnc(N)c(OC(F)(F)F)c4)cc32)C1)c1ccccc1Cl]

Example 297 was prepared in analogy to Example 31 using (rac)-5-[5′,6′-dihydrospiro[pyrrolidine-3,4′-pyrrolo[1,2-b]pyrazol]-2′-yl]-3-(trifluoromethoxy)pyridin-2-amine (75.0 mg, 221 μmol) and 2-(2-chlorophenyl)propan-2-amine-hydrochloride salt (54.7 mg, 265 μmol).

LC-MS (method 1): Rₜ=1.20 min; MS (ESIneg): m/z=533 [M−H]⁻

¹H-NMR (400 MHz, DMSO-d6) δ [ppm]: 1.520 (0.85), 1.681 (16.00), 1.693 (15.12), 1.902 (1.48), 2.014 (0.46), 2.028 (1.19), 2.043 (2.38), 2.061 (2.76), 2.073 (2.52), 2.078 (1.16), 2.091 (0.58), 2.518 (3.35), 2.523 (3.57), 2.542 (0.47), 2.849 (0.45), 2.887 (0.46), 2.900 (0.41), 3.413 (8.38), 3.440 (1.06), 3.483 (0.76), 3.502 (1.35), 3.516 (1.00), 3.526 (0.88), 3.542 (0.47), 4.121 (0.41), 4.145 (0.43), 4.155 (2.76), 4.173 (5.77), 4.191 (2.68), 6.121 (5.87), 6.382 (12.45), 6.418 (1.35), 6.478 (0.89), 6.516 (8.31), 7.157 (1.11), 7.162 (1.18), 7.176 (2.70), 7.180 (2.85), 7.195 (2.52), 7.199 (2.48), 7.227 (1.80), 7.231 (2.32), 7.247 (2.43), 7.250 (3.08), 7.265 (1.50), 7.269 (1.46), 7.278 (4.70), 7.282 (4.03), 7.297 (3.16), 7.301 (2.75), 7.457 (2.84), 7.461 (2.96), 7.476 (2.48), 7.480 (2.36), 7.732 (3.30), 7.735 (4.60), 7.739 (3.20), 8.317 (7.48), 8.322 (6.90).

### Example 298

### 2-[6-amino-5-(trifluoromethoxy)pyridin-3-yl]-N-[(1R)-1-(4-cyanopyridin-2-yl)ethyl]-6,7-dihydrospiro[pyrazolo[5,1-c][1,4]oxazine-4,3′-pyrrolidine]-1′-carboxamide (Mixture of Stereoisomers)

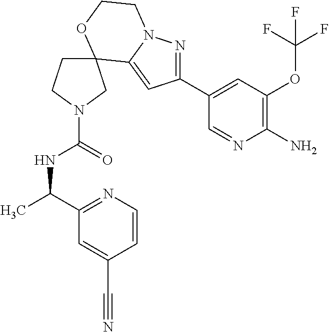 [SMILES: C[C@@H](NC(=O)N1CCC2(C1)OCCn1nc(-c3cnc(N)c(OC(F)(F)F)c3)cc12)c1cc(C#N)ccn1]

Example 298 was prepared in analogy to Example 31 using (rac)-5-[6,7-dihydrospiro[pyrazolo[5,1-c][1,4]oxazine-4,3′-pyrrolidin]-2-yl]-3-(trifluoromethoxy)pyridin-2-amine (75.0 mg, 211 μmol) and 2-[(1R)-1-aminoethyl]pyridine-4-carbonitrile (37.3 mg, 253 μmol).

LC-MS (method 1): Rₜ=0.93 min; MS (ESIpos): m/z=529 [M+H]⁺

¹H-NMR (400 MHz, DMSO-d6) δ [ppm]: 1.066 (0.87), 1.126 (0.44), 1.144 (0.47), 1.156 (0.46), 1.167 (0.44), 1.183 (0.41), 1.195 (0.51), 1.211 (0.52), 1.226 (1.52), 1.241 (5.73), 1.258 (10.11), 1.274 (4.94), 1.406 (13.87), 1.424 (13.88), 1.907 (1.05), 2.084 (10.64), 2.242 (1.12), 2.323 (1.87), 2.327 (2.77), 2.332 (2.44), 2.336 (1.85), 2.518 (6.12), 2.523 (4.14), 2.539 (16.00), 2.665 (1.31), 2.669 (1.84), 2.673 (1.32), 3.122 (0.65), 3.133 (0.66), 3.141 (0.63), 3.152 (0.62), 3.183 (0.80), 3.376 (1.75), 3.393 (1.43), 3.424 (1.13), 3.450 (1.54), 3.479 (1.33), 3.508 (1.14), 3.538 (1.42), 3.571 (1.46), 3.593 (1.01), 3.610 (0.75), 3.620 (1.41), 3.642 (1.48), 3.668 (0.65), 3.758 (1.70), 3.784 (1.40), 3.826 (1.65), 3.855 (1.39), 4.078 (1.80), 4.112 (3.96), 4.118 (4.23), 4.131 (7.42), 4.889 (1.16), 4.907 (1.93), 4.920 (2.00), 4.938 (1.21), 6.559 (7.78), 6.628 (1.77), 6.648 (2.03), 6.654 (2.12), 6.674 (1.73), 6.705 (5.95), 6.717 (7.50), 7.741 (5.41), 7.745 (5.24), 8.257 (2.55), 8.262 (6.61), 8.267 (6.54), 8.272 (2.50), 8.313 (5.59), 8.319 (9.96), 8.324 (6.22), 8.841 (5.12), 8.846 (7.55), 8.850 (4.86), 8.882 (5.67), 8.886 (7.48), 8.889 (5.49).

### Example 299

### (rac)-2-[6-amino-5-(difluoromethoxy)pyridin-3-yl]-N-[1-(2-chlorophenyl)cyclobutyl]-6,7-dihydrospiro[pyrazolo[5,1-c][1,4]oxazine-4,3′-pyrrolidine]-1′-carboxamide

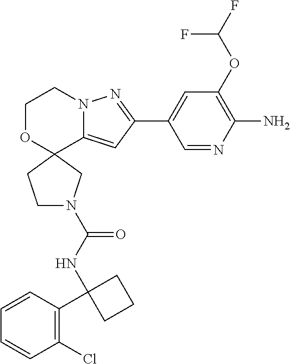 [SMILES: Nc1ncc(-c2cc3n(n2)CCOC32CCN(C(=O)NC3(c4ccccc4Cl)CCC3)C2)cc1OC(F)F]

Example 299 was prepared in analogy to Example 31 using (rac)-3-(difluoromethoxy)-5-[6,7-dihydrospiro[pyrazolo[5,1-c][1,4]oxazine-4,3′-pyrrolidin]-2-yl]pyridin-2-amine-hydrochloride salt (75.0 mg, 201 μmol) and 1-(2-chlorophenyl)cyclobutan-1-amine-hydrochloride salt (52.5 mg, 241 μmol).

¹H-NMR (400 MHz, DMSO-d6) δ [ppm]: 1.137 (1.02), 1.232 (1.23), 1.676 (0.80), 1.691 (0.84), 1.702 (0.92), 1.713 (0.65), 1.724 (0.41), 2.030 (0.64), 2.051 (1.18), 2.078 (1.24), 2.084 (16.00), 2.099 (0.70), 2.116 (0.54), 2.194 (0.67), 2.219 (0.93), 2.243 (0.54), 2.275 (0.85), 2.291 (1.01), 2.318 (1.29), 2.322 (2.57), 2.327 (3.40), 2.332 (2.38), 2.336 (1.01), 2.518 (10.90), 2.523 (7.42), 2.550 (1.09), 2.570 (0.50), 2.609 (0.83), 2.619 (1.16), 2.629 (1.62), 2.639 (1.65), 2.649 (1.61), 2.660 (2.22), 2.665 (2.79), 2.669 (3.79), 2.673 (2.63), 2.678 (1.43), 2.720 (0.42), 3.411 (1.16), 3.440 (1.31), 3.497 (0.74), 3.520 (1.28), 3.542 (0.56), 3.713 (1.35), 3.742 (1.13), 4.019 (0.55), 4.033 (0.97), 4.050 (1.41), 4.068 (1.45), 4.080 (1.74), 4.089 (2.05), 4.108 (4.48), 6.214 (7.08), 6.532 (4.94), 6.632 (7.08), 6.989 (2.96), 7.173 (6.53), 7.180 (1.42), 7.193 (2.90), 7.199 (2.80), 7.212 (2.85), 7.217 (2.53), 7.229 (2.13), 7.233 (2.66), 7.247 (2.85), 7.252 (3.36), 7.266 (1.46), 7.270 (1.31), 7.298 (4.37), 7.302 (4.14), 7.317 (2.82), 7.321 (2.70), 7.357 (2.71), 7.521 (2.78), 7.526 (2.75), 7.540 (2.58), 7.545 (2.36), 7.596 (3.87), 8.166 (5.90), 8.170 (6.03).

### Example 300

### (rac)-2-[6-amino-5-(trifluoromethoxy)pyridin-3-yl]-N-[1-(pyridin-2-yl)cyclobutyl]-6,7-dihydrospiro[pyrazolo[5,1-c][1,4]oxazine-4,3′-pyrrolidine]-1′-carboxamide

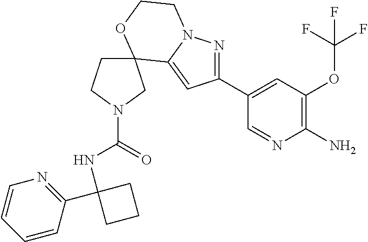 [SMILES: Nc1ncc(-c2cc3n(n2)CCOC32CCN(C(=O)NC3(c4ccccn4)CCC3)C2)cc1OC(F)(F)F]

Example 300 was prepared in analogy to Example 31 using (rac)-5-[6,7-dihydrospiro[pyrazolo[5,1-c][1,4]oxazine-4,3′-pyrrolidin]-2-yl]-3-(trifluoromethoxy)pyridin-2-amine (75.0 mg, 211 μmol) and 1-(pyridin-2-yl)cyclobutan-1-amine (37.5 mg, 253 μmol).

LC-MS (method 1): Rₜ=1.00 min; MS (ESIneg): m/z=528 [M−H]⁻

¹H-NMR (400 MHz, DMSO-d6) δ [ppm]: 1.066 (0.92), 1.898 (1.99), 1.983 (1.89), 2.007 (1.64), 2.075 (4.06), 2.085 (1.06), 2.231 (1.73), 2.264 (2.05), 2.323 (3.95), 2.327 (5.92), 2.332 (5.66), 2.336 (5.12), 2.358 (4.73), 2.366 (4.46), 2.382 (4.77), 2.406 (2.13), 2.454 (1.41), 2.458 (2.01), 2.463 (2.41), 2.518 (15.20), 2.523 (10.36), 2.535 (1.56), 2.539 (1.93), 2.545 (1.50), 2.568 (2.66), 2.590 (3.16), 2.613 (2.11), 2.665 (3.72), 2.669 (5.28), 2.673 (3.62), 2.728 (0.50), 2.888 (0.60), 2.942 (0.65), 2.971 (0.73), 3.098 (0.83), 3.289 (1.60), 3.413 (1.70), 3.499 (2.10), 3.528 (2.23), 3.634 (2.49), 3.810 (2.84), 3.838 (2.45), 4.057 (1.73), 4.068 (2.18), 4.086 (2.48), 4.105 (4.14), 4.131 (8.01), 4.143 (8.35), 4.161 (2.65), 6.544 (3.79), 6.559 (15.00), 6.623 (4.16), 6.729 (16.00), 6.810 (8.18), 7.169 (3.79), 7.173 (3.98), 7.181 (3.80), 7.185 (4.24), 7.188 (4.28), 7.191 (4.17), 7.200 (3.98), 7.203 (4.08), 7.352 (6.72), 7.372 (7.46), 7.677 (4.16), 7.682 (4.42), 7.696 (5.14), 7.701 (5.14), 7.716 (3.19), 7.720 (3.37), 7.744 (7.32), 7.749 (8.68), 8.177 (1.00), 8.326 (13.50), 8.331 (13.46), 8.523 (4.37), 8.526 (4.96), 8.528 (4.96), 8.530 (4.56), 8.535 (4.39), 8.538 (5.15), 8.541 (4.54).

### Example 301

### 2-[6-amino-5-(trifluoromethoxy)pyridin-3-yl]-N-[(1R)-1-(2-cyanopyridin-4-yl)ethyl]-6,7-dihydrospiro[pyrazolo[5,1-c][1,4]oxazine-4,3′-pyrrolidine]-1′-carboxamide (Mixture of Stereoisomers)

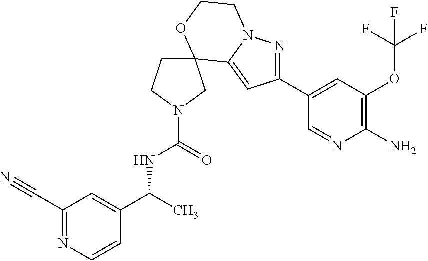 [SMILES: C[C@@H](NC(=O)N1CCC2(C1)OCCn1nc(-c3cnc(N)c(OC(F)(F)F)c3)cc12)c1ccnc(C#N)c1]

Example 301 was prepared in analogy to Example 31 using (rac)-5-[−6,7-dihydrospiro[pyrazolo[5,1-c][1,4]oxazine-4,3′-pyrrolidin]-2-yl]-3-(trifluoromethoxy)pyridin-2-amine (75.0 mg, 211 μmol) and 4-[(1R)-1-aminoethyl]pyridine-2-carbonitrile (37.3 mg, 253 μmol).

LC-MS (method 1): Rₜ=0.95 min; MS (ESIneg): m/z=527 [M−H]⁻

¹H-NMR (400 MHz, DMSO-d6) δ [ppm]: 1.065 (1.20), 1.140 (0.43), 1.157 (0.43), 1.169 (0.42), 1.186 (0.45), 1.231 (0.64), 1.374 (15.97), 1.392 (16.00), 2.074 (2.57), 2.083 (1.10), 2.228 (1.19), 2.253 (1.42), 2.322 (1.01), 2.327 (1.54), 2.332 (1.51), 2.337 (1.36), 2.351 (1.64), 2.518 (3.29), 2.523 (2.11), 2.665 (0.83), 2.669 (1.15), 2.673 (0.78), 2.727 (1.88), 2.888 (2.29), 3.355 (1.03), 3.378 (0.88), 3.391 (1.08), 3.408 (1.36), 3.417 (1.21), 3.427 (1.12), 3.444 (1.08), 3.461 (1.38), 3.491 (1.39), 3.521 (1.07), 3.551 (1.39), 3.584 (1.55), 3.606 (0.66), 3.641 (0.77), 3.663 (1.42), 3.686 (0.66), 3.772 (1.88), 3.800 (1.50), 3.841 (1.76), 3.868 (1.50), 4.053 (0.62), 4.066 (1.31), 4.083 (2.04), 4.098 (2.68), 4.119 (4.84), 4.126 (5.93), 4.138 (8.18), 4.840 (1.37), 4.858 (2.28), 4.872 (2.37), 4.889 (1.41), 6.558 (12.93), 6.673 (1.99), 6.692 (2.13), 6.712 (7.85), 6.722 (9.17), 7.675 (2.51), 7.680 (2.83), 7.682 (2.92), 7.687 (4.68), 7.693 (3.13), 7.695 (2.77), 7.700 (2.50), 7.744 (5.39), 7.748 (5.25), 8.003 (7.10), 8.320 (6.18), 8.325 (10.27), 8.331 (6.44), 8.672 (4.38), 8.674 (4.60), 8.677 (4.73), 8.678 (4.41), 8.685 (4.16), 8.687 (4.41), 8.689 (4.48).

### Example 302

### (rac)-2-[6-amino-5-(difluoromethoxy)pyridin-3-yl]-N-[2-(2-chlorophenyl)propan-2-yl]-6,7-dihydrospiro[pyrazolo[5,1-c][1,4]oxazine-4,3′-pyrrolidine]-1′-carboxamide

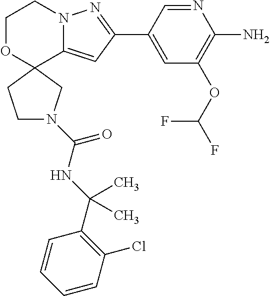 [SMILES: CC(C)(NC(=O)N1CCC2(C1)OCCn1nc(-c3cnc(N)c(OC(F)F)c3)cc12)c1ccccc1Cl]

Example 302 was prepared in analogy to Example 31 using (rac)-3-(difluoromethoxy)-5-[6,7-dihydrospiro[pyrazolo[5,1-c][1,4]oxazine-4,3′-pyrrolidin]-2-yl]pyridin-2-amine-hydrochloride salt (75.0 mg, 201 μmol) and 2-(2-chlorophenyl)propan-2-amine-hydrochloride salt (49.6 mg, 241 μmol).

LC-MS (method 1): Rt=1.12 min; MS (ESIpos): m/z=533 [M+H]⁺

¹H-NMR (400 MHz, DMSO-d6) δ [ppm]: 1.065 (1.27), 1.225 (1.56), 1.241 (5.97), 1.257 (9.69), 1.274 (4.86), 1.679 (8.91), 1.701 (8.39), 2.206 (0.56), 2.230 (0.80), 2.253 (0.45), 2.295 (0.63), 2.318 (1.11), 2.322 (1.86), 2.327 (2.56), 2.331 (1.93), 2.518 (16.00), 2.523 (11.15), 2.659 (0.73), 2.665 (1.53), 2.669 (2.18), 2.673 (1.62), 2.678 (0.77), 2.729 (0.95), 2.888 (1.17), 3.124 (0.68), 3.134 (0.66), 3.142 (0.69), 3.153 (0.67), 3.377 (0.80), 3.395 (0.83), 3.420 (0.52), 3.440 (1.36), 3.469 (1.59), 3.540 (0.80), 3.561 (1.25), 3.586 (0.91), 3.594 (0.87), 3.603 (0.81), 3.610 (0.91), 3.620 (0.92), 3.627 (0.81), 3.636 (0.79), 3.750 (1.52), 4.054 (0.48), 4.069 (0.85), 4.087 (0.98), 4.118 (2.76), 4.130 (3.58), 6.152 (3.43), 6.683 (6.02), 6.969 (1.24), 7.039 (1.32), 7.097 (1.48), 7.167 (0.67), 7.171 (0.78), 7.186 (1.65), 7.189 (1.65), 7.205 (1.60), 7.208 (1.55), 7.223 (3.38), 7.235 (1.27), 7.239 (1.47), 7.255 (1.56), 7.258 (1.83), 7.273 (0.92), 7.277 (0.85), 7.298 (2.67), 7.303 (2.38), 7.318 (2.02), 7.322 (1.80), 7.407 (1.21), 7.455 (1.72), 7.459 (1.78), 7.474 (1.49), 7.478 (1.38), 7.697 (2.00), 8.183 (4.23), 8.187 (3.97).

### Example 303

### (rac)-2-[6-amino-5-(trifluoromethoxy)pyridin-3-yl]-N-[1-(2-chlorophenyl)cyclobutyl]-6,7-dihydrospiro[pyrazolo[5,1-c][1,4]oxazine-4,3′-pyrrolidine]-1′-carboxamide

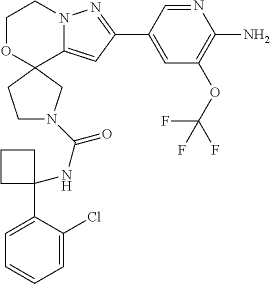 [SMILES: Nc1ncc(-c2cc3n(n2)CCOC32CCN(C(=O)NC3(c4ccccc4Cl)CCC3)C2)cc1OC(F)(F)F]

Example 303 was prepared in analogy to Example 31 using (rac)-5-[6,7-dihydrospiro[pyrazolo[5,1-c][1,4]oxazine-4,3′-pyrrolidin]-2-yl]-3-(trifluoromethoxy)pyridin-2-amine (75.0 mg, 211 μmol) and 1-(2-chlorophenyl)cyclobutan-1-amine-hydrochloride salt (55.2 mg, 253 μmol).

LC-MS (method 1): Rₜ=1.26 min; MS (ESIpos): m/z=563 [M+H]⁺

¹H-NMR (400 MHz, DMSO-d6) δ [ppm]: 1.036 (8.66), 1.054 (16.00), 1.071 (8.66), 1.230 (0.91), 1.653 (0.48), 1.674 (1.53), 1.686 (1.12), 1.690 (1.16), 1.702 (1.24), 1.712 (0.91), 1.723 (0.53), 2.030 (0.92), 2.052 (1.64), 2.073 (2.44), 2.078 (1.57), 2.099 (0.97), 2.155 (0.40), 2.186 (0.97), 2.212 (1.32), 2.236 (0.72), 2.277 (1.23), 2.292 (1.44), 2.309 (0.84), 2.323 (0.96), 2.327 (1.10), 2.444 (0.57), 2.467 (1.33), 2.474 (1.58), 2.518 (3.55), 2.522 (2.52), 2.540 (1.07), 2.550 (1.50), 2.571 (0.68), 2.609 (1.15), 2.620 (1.64), 2.630 (2.31), 2.639 (2.36), 2.649 (2.29), 2.660 (1.95), 2.669 (1.49), 2.679 (0.83), 2.729 (0.58), 2.887 (0.74), 3.166 (0.60), 3.412 (2.61), 3.430 (3.24), 3.447 (3.44), 3.464 (1.05), 3.501 (1.07), 3.522 (1.86), 3.545 (0.84), 3.714 (1.90), 3.743 (1.62), 4.018 (0.80), 4.033 (1.31), 4.049 (2.13), 4.056 (1.40), 4.069 (1.83), 4.080 (2.65), 4.089 (2.62), 4.111 (6.58), 6.536 (7.31), 6.554 (10.29), 6.671 (9.18), 7.173 (1.35), 7.178 (1.48), 7.192 (3.78), 7.196 (3.67), 7.211 (3.53), 7.215 (3.40), 7.228 (2.64), 7.231 (3.40), 7.246 (3.78), 7.250 (4.25), 7.265 (1.83), 7.269 (1.57), 7.295 (5.65), 7.299 (5.27), 7.314 (3.74), 7.318 (3.49), 7.522 (3.71), 7.527 (3.68), 7.541 (3.40), 7.546 (3.13), 7.720 (3.80), 7.725 (5.12), 7.728 (3.62), 8.295 (7.79), 8.300 (7.49).

### Example 304

### (rac)-2-[6-amino-5-(trifluoromethoxy)pyridin-3-yl]-N-[1-(2-fluorophenyl)cyclobutyl]-6,7-dihydrospiro[pyrazolo[5,1-c][1,4]oxazine-4,3′-pyrrolidine]-1′-carboxamide

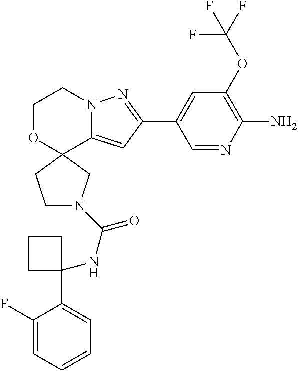 [SMILES: Nc1ncc(-c2cc3n(n2)CCOC32CCN(C(=O)NC3(c4ccccc4F)CCC3)C2)cc1OC(F)(F)F]

Example 304 was prepared in analogy to Example 31 using (rac)-5-[6,7-dihydrospiro[pyrazolo[5,1-c][1,4]oxazine-4,3′-pyrrolidin]-2-yl]-3-(trifluoromethoxy)pyridin-2-amine (75.0 mg, 211 μmol) and 1-(2-fluorophenyl)cyclobutan-1-amine-hydrochloride salt (51.1 mg, 253 μmol).

LC-MS (method 1): Rₜ=1.20 min; MS (ESIpos): m/z=547 [M+H]⁺

¹H-NMR (400 MHz, DMSO-d6) δ [ppm]: 1.678 (1.45), 1.700 (1.42), 1.726 (0.46), 1.732 (0.46), 1.739 (0.48), 1.746 (0.42), 1.753 (0.49), 2.032 (0.55), 2.055 (0.56), 2.059 (0.69), 2.072 (16.00), 2.211 (0.55), 2.287 (0.56), 2.302 (0.67), 2.322 (0.53), 2.326 (0.50), 2.331 (0.50), 2.518 (2.84), 2.522 (2.60), 2.539 (2.61), 3.414 (1.28), 3.443 (1.25), 3.471 (0.43), 3.515 (0.53), 3.537 (0.94), 3.559 (0.56), 3.726 (0.91), 3.754 (0.81), 4.037 (0.52), 4.054 (0.81), 4.073 (0.84), 4.084 (1.08), 4.094 (1.22), 4.114 (2.88), 6.153 (0.56), 6.554 (4.41), 6.600 (2.36), 6.683 (3.84), 6.690 (1.46), 7.041 (0.65), 7.043 (0.72), 7.061 (1.91), 7.072 (1.78), 7.091 (2.96), 7.109 (1.18), 7.112 (0.92), 7.196 (0.46), 7.200 (0.57), 7.208 (0.60), 7.214 (0.78), 7.220 (0.65), 7.228 (0.62), 7.233 (0.88), 7.252 (0.52), 7.294 (0.46), 7.404 (0.65), 7.408 (0.66), 7.427 (1.06), 7.445 (0.64), 7.727 (1.51), 7.731 (2.06), 7.735 (1.64), 8.301 (2.95), 8.306 (3.02), 8.325 (0.72), 8.330 (0.70).

### Example 305

### (rac)-2-[6-amino-5-(trifluoromethoxy)pyridin-3-yl]-N-[2-(3-chloropyridin-4-yl)propan-2-yl]-6,7-dihydrospiro[pyrazolo[5,1-c][1,4]oxazine-4,3′-pyrrolidine]-1′-carboxamide

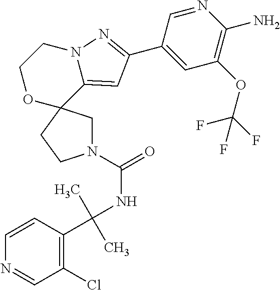 [SMILES: CC(C)(NC(=O)N1CCC2(C1)OCCn1nc(-c3cnc(N)c(OC(F)(F)F)c3)cc12)c1ccncc1Cl]

Example 305 was prepared in analogy to Example 31 using (rac)-5-[6,7-dihydrospiro[pyrazolo[5,1-c][1,4]oxazine-4,3′-pyrrolidin]-2-yl]-3-(trifluoromethoxy)pyridin-2-amine (75.0 mg, 211 μmol) and 2-(3-chloropyridin-4-yl)propan-2-amine (43.2 mg, 253 μmol).

LC-MS (method 1): Rₜ=1.03 min; MS (ESIpos): m/z=552 [M+H]⁺

¹H-NMR (400 MHz, DMSO-d6) δ [ppm]: 1.066 (1.27), 1.115 (0.44), 1.130 (0.58), 1.170 (2.00), 1.186 (2.09), 1.199 (2.26), 1.215 (2.79), 1.229 (2.54), 1.648 (16.00), 1.664 (15.30), 2.200 (0.95), 2.225 (1.32), 2.249 (0.70), 2.300 (1.06), 2.317 (1.51), 2.323 (1.55), 2.327 (1.79), 2.331 (1.56), 2.518 (9.13), 2.523 (6.29), 2.539 (1.91), 2.665 (0.87), 2.669 (1.12), 2.673 (0.83), 2.728 (0.68), 2.888 (0.82), 3.388 (1.32), 3.412 (0.71), 3.442 (1.74), 3.471 (2.05), 3.537 (0.90), 3.556 (1.55), 3.579 (0.73), 3.741 (1.76), 3.770 (1.50), 4.049 (0.65), 4.064 (1.30), 4.079 (1.61), 4.101 (2.42), 4.114 (4.46), 4.127 (6.52), 6.357 (5.88), 6.557 (8.40), 6.701 (10.85), 7.422 (4.73), 7.435 (4.82), 7.742 (3.34), 7.745 (4.39), 8.325 (6.79), 8.330 (6.65), 8.397 (6.78), 8.410 (6.60), 8.429 (13.24).

### Example 306

### (rac)-2′-[6-amino-5-(trifluoromethoxy)pyridin-3-yl]-N-[1-(pyridin-4-yl)cyclobutyl]-5′,6′-dihydro-1H-spiro[pyrrolidine-3,4′-pyrrolo[1,2-b]pyrazole]-1-carboxamide acetate (1:1)

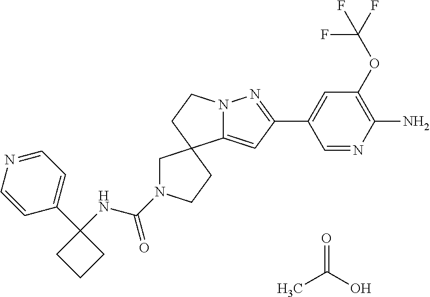

Example 306 was prepared in analogy to Example 31 using (rac)-5-[5′,6′-dihydrospiro[pyrrolidine-3,4′-pyrrolo[1,2-b]pyrazol]-2′-yl]-3-(trifluoromethoxy)pyridin-2-amine (75.0 mg, 221 μmol) and 1-(pyridin-4-yl)cyclobutan-1-amine (39.3 mg, 265 μmol).

LC-MS (method 2): Rt=0.70 min; MS (ESIpos): m/z=574 [M+H]⁺1H-NMR (400 MHz, DMSO-d6) δ [ppm]: 1.136 (0.77), 1.153 (0.78), 1.820 (0.44), 1.825 (0.47), 1.842 (0.50), 1.847 (0.57), 1.999 (0.55), 2.014 (0.62), 2.027 (0.66), 2.043 (0.83), 2.058 (1.16), 2.075 (1.28), 2.083 (0.71), 2.092 (0.58), 2.344 (0.44), 2.375 (1.07), 2.390 (1.20), 2.412 (1.13), 2.442 (1.08), 2.463 (0.92), 2.522 (1.21), 2.530 (1.88), 2.534 (1.86), 2.550 (1.19), 3.415 (1.02), 3.527 (0.48), 3.544 (0.73), 4.166 (1.43), 4.183 (2.70), 4.201 (1.46), 4.287 (0.61), 5.756 (16.00), 6.407 (4.57), 6.513 (4.45), 6.872 (2.23), 7.392 (2.26), 7.407 (2.38), 7.747 (1.57), 7.751 (2.20), 7.755 (1.69), 8.143 (2.56), 8.326 (2.98), 8.331 (3.02), 8.463 (1.48), 8.473 (1.47).

### Example 307

### (rac)-2-[6-amino-5-(trifluoromethoxy)pyridin-3-yl]-N-[(1-methyl-2-oxabicyclo[2.1.1]hexan-4-yl)methyl]-6,7-dihydrospiro[pyrazolo[5,1-c][1,4]oxazine-4,3′-pyrrolidine]-1′-carboxamide

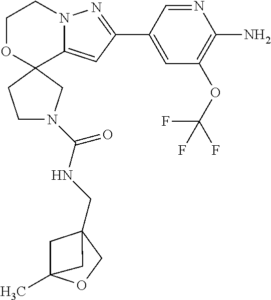 [SMILES: CC12CC(CNC(=O)N3CCC4(C3)OCCn3nc(-c5cnc(N)c(OC(F)(F)F)c5)cc34)(CO1)C2]

Example 307 was prepared in analogy to Example 31 using (rac)-5-[6,7-dihydrospiro[pyrazolo[5,1-c][1,4]oxazine-4,3′-pyrrolidin]-2-yl]-3-(trifluoromethoxy)pyridin-2-amine-hydrochloride salt (75.0 mg, 191 μmol) and 1-(1-methyl-2-oxabicyclo[2.1.1]hexan-4-yl)methanamine (29.2 mg, 230 μmol).

LC-MS (method 1): Rₜ=0.91 min; MS (ESIneg): m/z=507 [M−H]⁻

¹H-NMR (400 MHz, DMSO-d6) δ [ppm]: 1.139 (0.68), 1.155 (0.69), 1.243 (0.66), 1.247 (0.45), 1.260 (0.97), 1.277 (0.69), 1.298 (16.00), 1.316 (0.49), 1.338 (2.67), 1.342 (2.72), 1.349 (3.07), 1.353 (3.10), 1.375 (0.40), 1.482 (2.70), 1.487 (2.29), 1.493 (2.42), 2.213 (0.51), 2.238 (0.71), 2.314 (0.61), 2.328 (0.87), 2.346 (0.45), 3.294 (0.42), 3.405 (0.88), 3.431 (1.79), 3.462 (10.58), 3.528 (0.65), 3.549 (1.12), 3.571 (0.50), 3.759 (1.20), 3.787 (1.03), 4.056 (0.66), 4.072 (0.94), 4.090 (0.99), 4.102 (1.11), 4.111 (1.52), 4.127 (3.14), 6.258 (0.68), 6.272 (1.35), 6.287 (0.67), 6.577 (1.32), 6.699 (5.78), 7.747 (1.85), 7.750 (2.45), 7.754 (1.72), 8.321 (3.77), 8.326 (3.67).

### Example 308

### (rac)-2-[6-amino-5-(trifluoromethoxy)pyridin-3-yl]-N-[2-(2-fluorophenyl)propan-2-yl]-6,7-dihydrospiro[pyrazolo[5,1-c][1,4]oxazine-4,3′-pyrrolidine]-1′-carboxamide

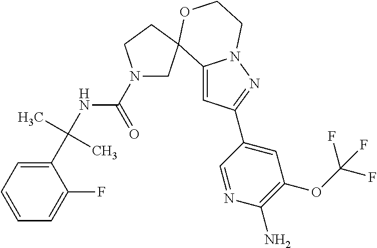 [SMILES: CC(C)(NC(=O)N1CCC2(C1)OCCn1nc(-c3cnc(N)c(OC(F)(F)F)c3)cc12)c1ccccc1F]

Example 308 was prepared in analogy to Example 31 using (rac)-5-[6,7-dihydrospiro[pyrazolo[5,1-c][1,4]oxazine-4,3′-pyrrolidin]-2-yl]-3-(trifluoromethoxy)pyridin-2-amine (75.0 mg, 211 μmol) and 2-(2-fluorophenyl)propan-2-amine-hydrochloride salt (48.0 mg, 253 μmol).

LC-MS (method 1): Rₜ=1.16 min; MS (ESIpos): m/z=535 [M+H]⁺

¹H-NMR (400 MHz, DMSO-d6) δ [ppm]: 1.240 (1.25), 1.255 (1.46), 1.270 (0.91), 1.628 (16.00), 1.635 (15.80), 2.074 (0.56), 2.084 (1.25), 2.219 (0.91), 2.245 (1.27), 2.268 (0.67), 2.323 (2.61), 2.327 (2.80), 2.523 (9.40), 2.665 (1.45), 2.669 (1.98), 2.728 (2.01), 2.888 (2.41), 3.401 (1.66), 3.468 (1.88), 3.497 (2.21), 3.563 (1.04), 3.585 (1.80), 3.771 (2.04), 3.801 (1.71), 4.063 (0.68), 4.077 (1.33), 4.094 (1.83), 4.124 (5.29), 4.135 (5.53), 6.117 (5.67), 6.561 (5.93), 6.714 (11.13), 7.035 (1.52), 7.055 (1.89), 7.067 (1.58), 7.075 (1.76), 7.079 (1.77), 7.087 (2.32), 7.094 (3.69), 7.113 (2.43), 7.116 (2.00), 7.195 (1.06), 7.209 (1.78), 7.228 (1.43), 7.246 (0.68), 7.299 (1.48), 7.320 (2.34), 7.340 (1.30), 7.743 (3.27), 7.747 (4.36), 8.324 (7.16), 8.328 (6.72).

### Example 309

### 2-[6-amino-5-(trifluoromethoxy)pyridin-3-yl]-N-[(1R)-1-(2-chloro-4-cyanophenyl)ethyl]-6,7-dihydrospiro[pyrazolo[5,1-c][1,4]oxazine-4,3′-pyrrolidine]-1′-carboxamide (Mixture of Stereoisomers)

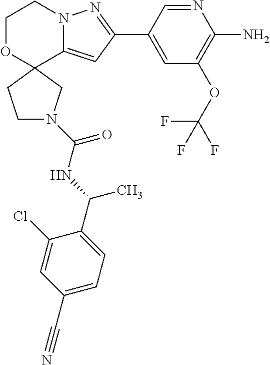 [SMILES: C[C@@H](NC(=O)N1CCC2(C1)OCCn1nc(-c3cnc(N)c(OC(F)(F)F)c3)cc12)c1ccc(C#N)cc1Cl]

Example 309 was prepared in analogy to Example 31 using (rac)-5-[6,7-dihydrospiro[pyrazolo[5,1-c][1,4]oxazine-4,3′-pyrrolidin]-2-yl]-3-(trifluoromethoxy)pyridin-2-amine (75.0 mg, 211 μmol) and 4-[(1R)-1-aminoethyl]-3-chlorobenzonitrile (45.8 mg, 253 μmol).

LC-MS (method 1): Rₜ=1.12 min; MS (ESIpos): m/z=562 [M+H]⁺

¹H-NMR (400 MHz, DMSO-d6) δ [ppm]: 1.229 (0.72), 1.328 (15.71), 1.346 (16.00), 2.074 (1.81), 2.083 (1.79), 2.250 (1.97), 2.327 (3.37), 2.331 (3.11), 2.522 (14.82), 2.669 (2.81), 3.264 (0.54), 3.286 (0.95), 3.292 (1.06), 3.308 (2.04), 3.370 (2.52), 3.381 (2.00), 3.398 (2.22), 3.423 (1.92), 3.447 (1.93), 3.472 (1.67), 3.511 (1.44), 3.542 (1.66), 3.575 (1.63), 3.659 (1.58), 3.766 (1.92), 3.794 (1.70), 3.840 (2.10), 3.871 (1.82), 4.075 (2.82), 4.092 (3.04), 4.137 (9.91), 5.116 (1.88), 5.125 (2.12), 5.134 (2.86), 5.142 (3.05), 5.152 (1.98), 5.159 (1.99), 6.559 (14.75), 6.715 (8.19), 6.723 (8.72), 6.785 (2.33), 6.803 (2.35), 6.823 (2.36), 6.842 (2.17), 7.662 (4.48), 7.676 (4.70), 7.682 (5.83), 7.696 (5.37), 7.746 (5.09), 7.750 (4.93), 7.823 (3.56), 7.826 (3.78), 7.833 (4.03), 7.837 (4.05), 7.842 (2.91), 7.846 (3.02), 7.853 (2.98), 7.858 (2.89), 7.991 (11.46), 7.995 (10.64), 8.316 (6.42), 8.321 (6.50), 8.328 (7.05), 8.333 (6.68).

### Example 310

### 2′-[6-amino-5-(difluoromethoxy)pyridin-3-yl]-N-[2-(pyridin-2-yl)propan-2-yl]-5′,6′-dihydrospiro[azetidine-3,4′-pyrrolo[1,2-b]pyrazole]-1-carboxamide

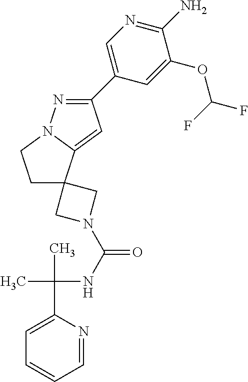 [SMILES: CC(C)(NC(=O)N1CC2(CCn3nc(-c4cnc(N)c(OC(F)F)c4)cc32)C1)c1ccccn1]

Example 310 was prepared in analogy to Example 31 using 3-(difluoromethoxy)-5-(5′,6′-dihydrospiro[azetidine-3,4′-pyrrolo[1,2-b]pyrazol]-2′-yl)pyridin-2-amine (75.0 mg, 244 μmol) and 2-(pyridin-2-yl)propan-2-amine (39.9 mg, 293 μmol).

LC-MS (method 1): Rₜ=0.93 min; MS (ESIpos): m/z=470 [M+H]⁺

¹H-NMR (400 MHz, DMSO-d6) δ [ppm]: 1.054 (0.50), 1.570 (16.00), 2.836 (1.12), 2.853 (1.86), 2.871 (1.22), 3.167 (0.55), 4.017 (0.72), 4.038 (5.03), 4.042 (5.02), 4.063 (0.73), 4.110 (1.26), 4.128 (1.99), 4.145 (1.21), 6.171 (3.30), 6.637 (6.27), 6.721 (2.81), 7.007 (1.32), 7.180 (0.88), 7.183 (0.95), 7.191 (3.21), 7.195 (1.27), 7.199 (1.06), 7.201 (0.97), 7.211 (0.94), 7.213 (0.94), 7.375 (1.20), 7.442 (1.57), 7.444 (0.96), 7.462 (1.76), 7.656 (1.80), 7.658 (1.80), 7.717 (0.89), 7.721 (0.90), 7.736 (1.06), 7.740 (1.11), 7.756 (0.72), 7.760 (0.70), 8.252 (3.11), 8.256 (2.95), 8.458 (0.96), 8.461 (1.08), 8.463 (1.13), 8.465 (1.01), 8.470 (1.01), 8.473 (1.13), 8.475 (1.07), 8.477 (0.94).

### Example 311

### 2′-[6-amino-5-(difluoromethoxy)pyridin-3-yl]-N-[2-(1-methyl-1H-1,2,3-triazol-4-yl)propan-2-yl]-5′,6′-dihydrospiro[azetidine-3,4′-pyrrolo[1,2-b]pyrazole]-1-carboxamide

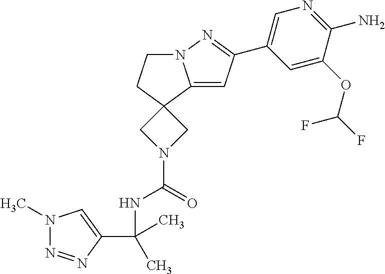 [SMILES: Cn1cc(C(C)(C)NC(=O)N2CC3(CCn4nc(-c5cnc(N)c(OC(F)F)c5)cc43)C2)nn1]

Example 311 was prepared in analogy to Example 31 using 3-(difluoromethoxy)-5-(5′,6′-dihydrospiro[azetidine-3,4′-pyrrolo[1,2-b]pyrazol]-2′-yl)pyridin-2-amine (75.0 mg, 244 μmol) and 2-(1-methyl-1H-1,2,3-triazol-4-yl)propan-2-amine-hydrochloride salt (62.4 mg, 293 μmol).

LC-MS (method 1): Rₜ=0.79 min; MS (ESIneg): m/z=472 [M−H]⁻

¹H-NMR (400 MHz, DMSO-d6) δ [ppm]: 1.243 (0.45), 1.261 (0.57), 1.587 (15.68), 2.563 (0.69), 2.728 (1.30), 2.805 (1.38), 2.822 (2.43), 2.839 (1.50), 2.888 (1.44), 3.993 (16.00), 4.097 (1.51), 4.115 (2.50), 4.131 (1.49), 6.254 (1.27), 6.404 (2.75), 6.622 (3.96), 7.012 (0.96), 7.195 (1.92), 7.380 (0.90), 7.665 (2.37), 7.803 (4.05), 8.236 (2.46), 8.240 (2.59).

### Example 312

### 2-[6-amino-5-(trifluoromethoxy)pyridin-3-yl]-N-[(1S)-2-methoxy-1-phenylethyl]-6,7-dihydrospiro[pyrazolo[5,1-c][1,4]oxazine-4,3′-pyrrolidine]-1′-carboxamide (Mixture of Stereoisomers)

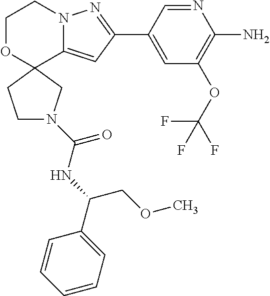 [SMILES: COC[C@@H](NC(=O)N1CCC2(C1)OCCn1nc(-c3cnc(N)c(OC(F)(F)F)c3)cc12)c1ccccc1]

Example 312 was prepared in analogy to Example 31 using (rac)-5-[6,7-dihydrospiro[pyrazolo[5,1-c][1,4]oxazine-4,3′-pyrrolidin]-2-yl]-3-(trifluoromethoxy)pyridin-2-amine (75.0 mg, 211 μmol) and (1S)-2-methoxy-1-phenylethan-1-amine (38.3 mg, 253 μmol).

LC-MS (method 1): Rₜ=1.08 min; MS (ESIpos): m/z=533 [M+H]⁺

¹H-NMR (400 MHz, DMSO-d6) δ [ppm]: 2.522 (0.95), 3.204 (16.00), 3.247 (0.72), 3.257 (6.57), 3.443 (0.52), 3.454 (2.98), 3.469 (2.70), 3.490 (0.49), 3.547 (0.48), 3.567 (0.65), 3.592 (0.49), 4.103 (0.41), 4.129 (0.99), 4.774 (0.78), 4.781 (0.48), 4.788 (0.49), 4.795 (0.80), 6.501 (0.42), 6.558 (1.47), 6.584 (1.21), 6.604 (1.17), 6.709 (1.41), 7.212 (0.51), 7.222 (0.79), 7.229 (1.38), 7.234 (0.72), 7.241 (0.75), 7.246 (0.88), 7.250 (0.67), 7.269 (1.27), 7.286 (3.73), 7.303 (3.24), 7.308 (1.59), 7.320 (2.60), 7.338 (0.97), 7.346 (1.36), 7.367 (0.61), 7.742 (0.68), 7.745 (0.68), 8.315 (0.60), 8.320 (1.14), 8.325 (0.66).

### Example 313

### 2-[6-amino-5-(trifluoromethoxy)pyridin-3-yl]-N-[(1S)-1-(3-chloropyridin-4-yl)ethyl]-6,7-dihydrospiro[pyrazolo[5,1-c][1,4]oxazine-4,3′-pyrrolidine]-1′-carboxamide (Mixture of Stereoisomers)

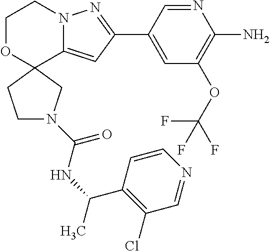 [SMILES: C[C@H](NC(=O)N1CCC2(C1)OCCn1nc(-c3cnc(N)c(OC(F)(F)F)c3)cc12)c1ccncc1Cl]

Example 313 was prepared in analogy to Example 31 using (rac)-5-[6,7-dihydrospiro[pyrazolo[5,1-c][1,4]oxazine-4,3′-pyrrolidin]-2-yl]-3-(trifluoromethoxy)pyridin-2-amine (75.0 mg, 211 μmol) and (1S)-1-(3-chloropyridin-4-yl)ethan-1-amine-hydrochloride salt (48.9 mg, 253 μmol).

LC-MS (method 1): Rₜ=0.99 min; MS (ESIpos): m/z=538 [M+H]⁺

¹H-NMR (400 MHz, DMSO-d6) δ [ppm]: 1.035 (4.87), 1.052 (10.54), 1.065 (2.30), 1.070 (5.47), 1.130 (0.44), 1.154 (0.95), 1.170 (1.58), 1.185 (1.63), 1.199 (1.62), 1.215 (2.07), 1.227 (2.21), 1.336 (10.41), 1.351 (10.41), 1.354 (10.45), 1.646 (0.76), 1.752 (0.47), 2.228 (1.09), 2.254 (1.26), 2.322 (0.68), 2.326 (0.99), 2.331 (1.13), 2.349 (1.41), 2.518 (4.83), 2.522 (3.40), 2.539 (1.54), 2.664 (0.51), 2.668 (0.66), 2.673 (0.51), 3.165 (1.17), 3.393 (1.16), 3.410 (1.64), 3.429 (2.22), 3.447 (2.20), 3.461 (1.76), 3.489 (1.12), 3.526 (0.88), 3.564 (15.07), 3.587 (1.21), 3.611 (0.55), 3.649 (0.64), 3.671 (1.14), 3.694 (0.54), 3.775 (1.45), 3.804 (1.20), 3.842 (1.44), 3.871 (1.25), 4.049 (0.56), 4.063 (1.27), 4.080 (2.01), 4.098 (2.39), 4.120 (3.97), 4.134 (6.31), 4.140 (6.76), 4.365 (0.42), 5.046 (1.29), 5.054 (1.48), 5.064 (2.03), 5.072 (2.18), 5.081 (1.41), 5.090 (1.38), 5.758 (0.47), 6.563 (9.81), 6.715 (5.55), 6.723 (6.38), 6.785 (1.73), 6.803 (1.73), 6.821 (1.67), 6.839 (1.54), 7.483 (3.32), 7.497 (3.83), 7.500 (3.70), 7.513 (3.36), 7.743 (3.36), 7.749 (3.34), 7.754 (3.33), 8.320 (4.68), 8.324 (4.57), 8.330 (4.93), 8.335 (4.85), 8.481 (4.12), 8.492 (6.67), 8.504 (4.50), 8.533 (16.00).

### Example 314

### (rac)-2-[6-amino-5-(trifluoromethoxy)pyridin-3-yl]-N-[2-(3-fluorophenyl)propan-2-yl]-6,7-dihydrospiro[pyrazolo[5,1-c][1,4]oxazine-4,3′-pyrrolidine]-1′-carboxamide

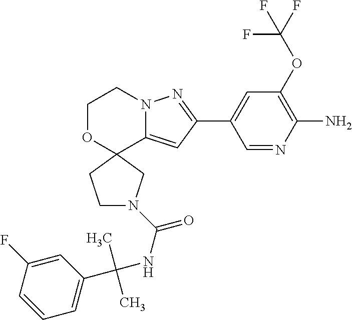 [SMILES: CC(C)(NC(=O)N1CCC2(C1)OCCn1nc(-c3cnc(N)c(OC(F)(F)F)c3)cc12)c1cccc(F)c1]

Example 314 was prepared in analogy to Example 31 using (rac)-5-[6,7-dihydrospiro[pyrazolo[5,1-c][1,4]oxazine-4,3′-pyrrolidin]-2-yl]-3-(trifluoromethoxy)pyridin-2-amine (75.0 mg, 211 μmol) and 2-(3-fluorophenyl)propan-2-amine (38.8 mg, 253 μmol).

LC-MS (method 1): Rₜ=1.17 min; MS (ESIneg): m/z=533 [M−H]⁻

¹H-NMR (400 MHz, DMSO-d6) δ [ppm]: 1.066 (5.27), 1.156 (3.40), 1.380 (0.51), 1.409 (0.72), 1.427 (0.70), 1.548 (16.00), 1.554 (15.66), 2.084 (0.92), 2.219 (0.77), 2.245 (1.03), 2.268 (0.58), 2.310 (0.90), 2.323 (1.56), 2.327 (1.93), 2.359 (0.52), 2.518 (3.63), 2.523 (2.51), 2.665 (0.66), 2.669 (0.91), 2.673 (0.64), 3.365 (0.62), 3.392 (0.90), 3.409 (0.90), 3.434 (0.45), 3.482 (1.14), 3.510 (1.33), 3.578 (0.76), 3.601 (1.25), 3.623 (0.59), 3.785 (1.52), 3.814 (1.29), 4.069 (0.45), 4.083 (1.01), 4.100 (1.73), 4.117 (3.56), 4.124 (4.93), 4.136 (4.76), 6.153 (4.85), 6.541 (1.32), 6.555 (6.83), 6.720 (9.24), 6.939 (0.85), 6.943 (0.94), 6.959 (1.67), 6.965 (1.83), 6.981 (0.90), 6.986 (1.00), 7.105 (1.27), 7.109 (1.71), 7.115 (1.36), 7.133 (1.24), 7.137 (1.73), 7.143 (1.34), 7.176 (1.93), 7.196 (2.53), 7.275 (1.52), 7.291 (1.71), 7.295 (2.37), 7.310 (2.26), 7.315 (1.18), 7.331 (0.97), 7.742 (2.72), 7.746 (3.74), 7.750 (2.55), 8.324 (5.97), 8.330 (5.94).

### Example 315

### (rac)-2′-[6-amino-5-(trifluoromethoxy)pyridin-3-yl]-N-[(1-methyl-2-oxabicyclo[2.1.1]hexan-4-yl)methyl]-5′,6′-dihydrospiro[pyrrolidine-3,4′-pyrrolo[1,2-b]pyrazole]-1-carboxamide

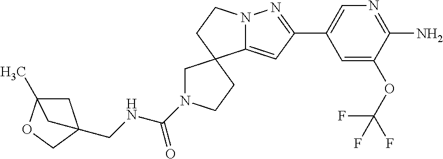 [SMILES: CC12CC(CNC(=O)N3CCC4(CCn5nc(-c6cnc(N)c(OC(F)(F)F)c6)cc54)C3)(CO1)C2]

Example 315 was prepared in analogy to Example 31 using (rac)-5-[5′,6′-dihydrospiro[pyrrolidine-3,4′-pyrrolo[1,2-b]pyrazol]-2′-yl]-3-(trifluoromethoxy)pyridin-2-amine-hydrochloride salt (75.0 mg, 200 μmol) and 1-(1-methyl-2-oxabicyclo[2.1.1]hexan-4-yl)methanamine (30.5 mg, 239 μmol).

LC-MS (method 1): Rₜ=0.91 min; MS (ESIneg): m/z=491 [M−H]⁻

¹H-NMR (400 MHz, DMSO-d6) δ [ppm]: 1.138 (0.52), 1.155 (0.52), 1.289 (9.42), 1.337 (1.48), 1.341 (1.50), 1.347 (1.68), 1.351 (1.73), 1.475 (0.86), 1.486 (1.42), 1.497 (0.77), 2.041 (0.43), 2.055 (0.70), 2.067 (0.85), 2.083 (1.12), 2.518 (1.67), 2.522 (1.51), 2.537 (0.86), 2.727 (1.14), 2.888 (1.40), 3.340 (16.00), 3.348 (2.04), 3.390 (0.49), 3.398 (0.43), 3.433 (0.42), 3.464 (5.17), 3.501 (0.50), 4.160 (0.92), 4.178 (1.67), 4.195 (0.91), 6.257 (0.78), 6.388 (3.62), 6.502 (2.57), 7.742 (0.98), 7.746 (1.32), 7.750 (0.92), 8.322 (2.08), 8.327 (2.09).

### Example 316

### 2′-[6-amino-5-(difluoromethoxy)pyridin-3-yl]-N-[(1R)-1-phenylpropyl]-5′,6′-dihydrospiro[azetidine-3,4′-pyrrolo[1,2-b]pyrazole]-1-carboxamide

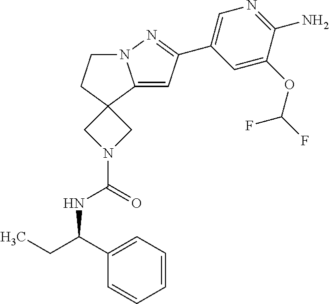 [SMILES: CC[C@@H](NC(=O)N1CC2(CCn3nc(-c4cnc(N)c(OC(F)F)c4)cc32)C1)c1ccccc1]

Example 316 was prepared in analogy to Example 31 using 3-(difluoromethoxy)-5-(5′,6′-dihydrospiro[azetidine-3,4′-pyrrolo[1,2-b]pyrazol]-2′-yl)pyridin-2-amine (75.0 mg, 244 μmol) and (1R)-1-phenylpropan-1-amine (39.6 mg, 293 μmol).

LC-MS (method 1): Rₜ=1.07 min; MS (ESIpos): m/z=469 [M+H]⁺

¹H-NMR (400 MHz, DMSO-d6) δ [ppm]: 0.846 (4.06), 0.864 (9.69), 0.882 (4.38), 1.644 (0.63), 1.662 (1.48), 1.672 (1.31), 1.679 (1.90), 1.693 (1.73), 1.711 (1.22), 1.727 (0.42), 2.729 (0.60), 2.817 (2.17), 2.835 (3.69), 2.852 (2.33), 2.886 (0.75), 3.986 (1.69), 4.007 (4.58), 4.015 (9.02), 4.027 (4.19), 4.048 (1.69), 4.097 (2.39), 4.114 (3.86), 4.131 (2.30), 4.512 (0.67), 4.534 (1.61), 4.550 (1.42), 4.572 (0.65), 6.162 (6.45), 6.609 (10.00), 6.814 (2.55), 6.836 (2.46), 6.997 (2.25), 7.181 (4.73), 7.192 (0.90), 7.199 (1.13), 7.206 (1.62), 7.214 (1.42), 7.221 (1.41), 7.228 (1.01), 7.286 (1.01), 7.291 (0.61), 7.307 (6.85), 7.312 (7.39), 7.320 (16.00), 7.333 (0.94), 7.366 (2.10), 7.642 (3.68), 8.232 (5.13), 8.236 (5.17).

### Example 317

### 2′-[6-amino-5-(trifluoromethoxy)pyridin-3-yl]-N-[(1S)-2-methoxy-1-phenylethyl]-5′,6′-dihydrospiro[pyrrolidine-3,4′-pyrrolo[1,2-b]pyrazole]-1-carboxamide (Mixture of Stereoisomers)

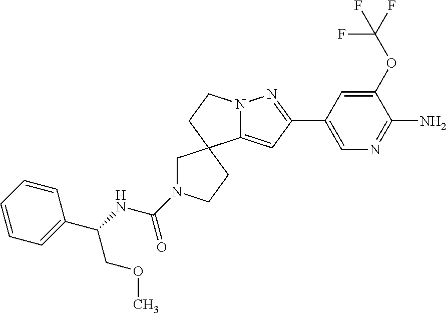 [SMILES: COC[C@@H](NC(=O)N1CCC2(CCn3nc(-c4cnc(N)c(OC(F)(F)F)c4)cc32)C1)c1ccccc1]

Example 317 was prepared in analogy to Example 31 using (rac)-5-[5′,6′-dihydrospiro[pyrrolidine-3,4′-pyrrolo[1,2-b]pyrazol]-2′-yl]-3-(trifluoromethoxy)pyridin-2-amine-hydrochloride salt (75.0 mg, 200 μmol) and (1S)-2-methoxy-1-phenylethan-1-amine (36.2 mg, 239 μmol).

LC-MS (method 1): Rₜ=1.06 min; MS (ESIpos): m/z=517 [M+H]⁺

¹H-NMR (400 MHz, DMSO-d6) δ [ppm]: 1.017 (0.62), 1.029 (0.88), 1.033 (0.85), 1.047 (0.89), 2.062 (1.86), 2.073 (2.85), 2.083 (4.54), 2.534 (3.74), 2.551 (2.36), 3.204 (5.09), 3.223 (0.48), 3.245 (15.36), 3.259 (16.00), 3.425 (1.51), 3.433 (1.48), 3.450 (3.65), 3.461 (5.26), 3.468 (4.00), 3.479 (2.63), 3.504 (0.87), 3.517 (0.43), 3.535 (0.75), 3.547 (1.53), 3.556 (1.55), 3.567 (1.89), 3.571 (1.76), 3.576 (1.88), 3.592 (1.21), 3.601 (0.88), 4.163 (2.08), 4.181 (3.69), 4.198 (2.05), 4.930 (0.62), 4.950 (1.42), 4.964 (1.44), 4.985 (0.67), 6.395 (7.26), 6.459 (1.17), 6.469 (1.36), 6.480 (1.33), 6.490 (1.52), 6.507 (6.48), 7.186 (0.50), 7.203 (1.69), 7.221 (2.02), 7.225 (1.25), 7.234 (0.82), 7.238 (1.09), 7.241 (0.76), 7.266 (1.63), 7.286 (4.07), 7.303 (3.42), 7.307 (3.59), 7.320 (1.68), 7.325 (2.08), 7.337 (3.24), 7.359 (3.52), 7.379 (1.59), 7.743 (2.80), 7.747 (2.90), 8.315 (2.67), 8.318 (4.06), 8.322 (3.05).

### Example 318

### 2′-[6-amino-5-(difluoromethoxy)pyridin-3-yl]-N-[(1R)-1-(3-fluorophenyl)ethyl]-5′,6′-dihydrospiro[azetidine-3,4′-pyrrolo[1,2-b]pyrazole]-1-carboxamide

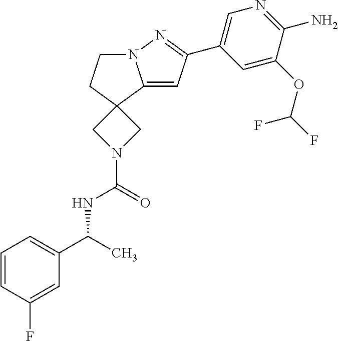 [SMILES: C[C@@H](NC(=O)N1CC2(CCn3nc(-c4cnc(N)c(OC(F)F)c4)cc32)C1)c1cccc(F)c1]

Example 318 was prepared in analogy to Example 31 using 3-(difluoromethoxy)-5-(5′,6′-dihydrospiro[azetidine-3,4′-pyrrolo[1,2-b]pyrazol]-2′-yl)pyridin-2-amine (75.0 mg, 244 μmol) and (1R)-1-(3-fluorophenyl)ethan-1-amine (40.8 mg, 293 μmol).

LC-MS (method 1): Rₜ=1.03 min; MS (ESIpos): m/z=473 [M+H]⁺

¹H-NMR (400 MHz, DMSO-d6) δ [ppm]: 1.234 (0.70), 1.245 (1.50), 1.253 (0.98), 1.261 (1.60), 1.266 (1.57), 1.271 (0.84), 1.283 (1.78), 1.302 (0.59), 1.347 (13.14), 1.365 (12.92), 2.074 (1.13), 2.518 (3.12), 2.523 (2.00), 2.534 (0.76), 2.539 (0.52), 2.727 (0.68), 2.828 (3.28), 2.846 (5.39), 2.863 (3.57), 2.888 (0.91), 3.824 (0.66), 3.998 (2.89), 4.019 (6.84), 4.027 (13.50), 4.046 (5.58), 4.066 (2.83), 4.103 (3.60), 4.121 (5.81), 4.137 (3.47), 4.782 (0.43), 4.800 (1.69), 4.820 (2.31), 4.838 (1.68), 4.856 (0.43), 6.285 (1.77), 6.436 (0.48), 6.637 (16.00), 6.680 (0.57), 6.918 (3.90), 6.939 (3.80), 7.011 (6.21), 7.031 (2.30), 7.038 (2.60), 7.054 (1.33), 7.058 (1.50), 7.060 (1.42), 7.138 (1.96), 7.148 (2.36), 7.154 (2.11), 7.164 (3.37), 7.175 (2.69), 7.181 (5.39), 7.195 (7.31), 7.204 (0.53), 7.266 (1.64), 7.330 (2.19), 7.345 (2.33), 7.350 (3.10), 7.366 (3.52), 7.369 (1.85), 7.379 (3.34), 7.386 (1.70), 7.667 (4.96), 7.695 (0.47), 7.698 (0.45), 8.228 (8.68), 8.233 (8.72).

### Example 319

### 2′-[6-amino-5-(trifluoromethoxy)pyridin-3-yl]-N-[(1S)-1-(pyridin-3-yl)ethyl]-5′,6′-dihydrospiro[pyrrolidine-3,4′-pyrrolo[1,2-b]pyrazole]-1-carboxamide (Mixture of Stereoisomers)

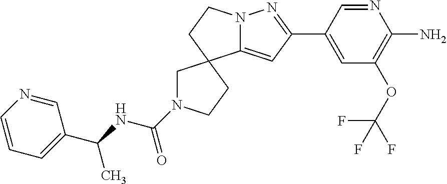 [SMILES: C[C@H](NC(=O)N1CCC2(CCn3nc(-c4cnc(N)c(OC(F)(F)F)c4)cc32)C1)c1cccnc1]

Example 319 was prepared in analogy to Example 31 using (rac)-5-[5′,6′-dihydrospiro[pyrrolidine-3,4′-pyrrolo[1,2-b]pyrazol]-2′-yl]-3-(trifluoromethoxy)pyridin-2-amine (75.0 mg, 221 μmol) and (1S)-1-(pyridin-3-yl)ethan-1-amine-hydrochloride salt (51.7 mg, 265 μmol).

LC-MS (method 1): Rₜ=0.61 min; MS (ESIpos): m/z=488 [M+H]⁺

¹H-NMR (400 MHz, DMSO-d6) δ [ppm]: 1.166 (0.50), 1.232 (1.15), 1.388 (12.62), 1.395 (12.76), 1.406 (13.14), 1.413 (12.34), 2.045 (2.96), 2.064 (4.60), 2.072 (4.54), 2.518 (8.38), 2.523 (7.14), 2.529 (9.66), 2.539 (2.61), 2.547 (6.32), 3.403 (1.66), 3.422 (3.89), 3.428 (2.60), 3.447 (9.62), 3.454 (12.05), 3.461 (8.77), 3.480 (1.70), 3.487 (1.84), 3.506 (1.13), 3.525 (1.87), 3.541 (1.86), 3.560 (1.92), 3.574 (1.28), 3.586 (1.16), 3.601 (0.65), 4.161 (5.68), 4.178 (9.95), 4.195 (5.41), 4.851 (1.86), 4.856 (2.09), 4.875 (2.94), 4.888 (2.04), 4.893 (1.89), 6.395 (7.43), 6.411 (14.61), 6.502 (16.00), 6.563 (3.27), 6.573 (3.40), 6.582 (3.38), 6.593 (2.98), 7.293 (2.14), 7.305 (2.32), 7.312 (4.65), 7.324 (4.74), 7.332 (2.67), 7.344 (2.54), 7.712 (1.71), 7.717 (2.83), 7.721 (1.85), 7.732 (1.75), 7.753 (8.44), 7.770 (1.74), 8.321 (8.40), 8.324 (10.53), 8.329 (8.74), 8.392 (3.69), 8.396 (3.99), 8.404 (6.53), 8.408 (6.26), 8.417 (3.90), 8.421 (3.66), 8.540 (4.53), 8.546 (4.61), 8.560 (4.68), 8.565 (4.64).

### Example 320

### (rac)-2′-[6-amino-5-(trifluoromethoxy)pyridin-3-yl]-N-[1-(pyridin-3-yl)cyclobutyl]-5′,6′-dihydrospiro[pyrrolidine-3,4′-pyrrolo[1,2-b]pyrazole]-1-carboxamide

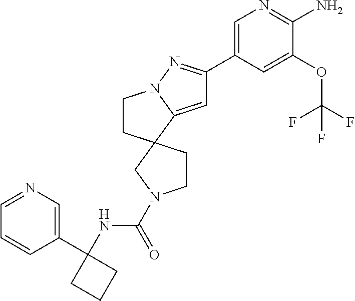 [SMILES: Nc1ncc(-c2cc3n(n2)CCC32CCN(C(=O)NC3(c4cccnc4)CCC3)C2)cc1OC(F)(F)F]

Example 320 was prepared in analogy to Example 31 using (rac)-5-[5′,6′-dihydrospiro[pyrrolidine-3,4′-pyrrolo[1,2-b]pyrazol]-2′-yl]-3-(trifluoromethoxy)pyridin-2-amine-hydrochloride salt (75.0 mg, 200 μmol) and 1-(pyridin-3-yl)cyclobutan-1-amine (35.5 mg, 239 μmol).

LC-MS (method 1): Rt=0.99 min; MS (ESIpos): m/z=514 [M+H]⁺

¹H-NMR (400 MHz, DMSO-d6) δ [ppm]: 0.852 (0.50), 0.917 (0.50), 0.932 (4.03), 0.935 (1.31), 0.948 (4.08), 0.953 (0.68), 1.232 (1.89), 1.765 (0.67), 1.787 (1.32), 1.793 (1.33), 1.815 (1.80), 1.832 (1.08), 1.854 (0.41), 1.967 (0.45), 1.989 (1.03), 2.004 (1.96), 2.011 (1.50), 2.020 (1.72), 2.032 (2.92), 2.047 (3.56), 2.062 (3.52), 2.078 (1.48), 2.318 (1.22), 2.397 (0.98), 2.405 (0.89), 2.426 (3.03), 2.447 (4.76), 2.465 (5.11), 2.518 (16.00), 2.523 (13.98), 2.539 (4.27), 2.660 (1.24), 3.358 (0.69), 3.399 (2.47), 3.425 (6.69), 3.435 (4.99), 3.461 (1.08), 3.505 (0.96), 3.523 (1.62), 3.564 (0.60), 4.159 (4.18), 4.177 (7.44), 4.194 (4.01), 6.376 (13.65), 6.507 (12.00), 6.828 (6.88), 7.291 (3.22), 7.293 (3.23), 7.302 (3.46), 7.304 (3.41), 7.310 (3.59), 7.312 (3.51), 7.322 (3.48), 7.324 (3.41), 7.746 (4.59), 7.750 (6.21), 7.754 (4.22), 7.782 (2.69), 7.785 (3.53), 7.792 (2.77), 7.801 (2.61), 7.807 (3.16), 7.811 (2.52), 8.319 (10.62), 8.324 (10.00), 8.364 (6.28), 8.368 (6.01), 8.376 (5.93), 8.380 (5.66), 8.640 (6.22), 8.642 (6.52), 8.646 (6.44).

### Example 321

### (rac)-2′-[6-amino-5-(trifluoromethoxy)pyridin-3-yl]-N-[1-(3-fluorophenyl)cyclobutyl]-5′,6′-dihydrospiro[pyrrolidine-3,4′-pyrrolo[1,2-b]pyrazole]-1-carboxamide

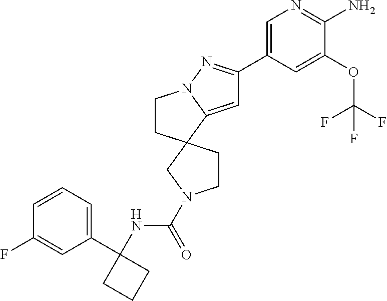 [SMILES: Nc1ncc(-c2cc3n(n2)CCC32CCN(C(=O)NC3(c4cccc(F)c4)CCC3)C2)cc1OC(F)(F)F]

Example 321 was prepared in analogy to Example 31 using (rac)-5-[5′,6′-dihydrospiro[pyrrolidine-3,4′-pyrrolo[1,2-b]pyrazol]-2′-yl]-3-(trifluoromethoxy)pyridin-2-amine-hydrochloride salt (75.0 mg, 200 μmol) and 1-(3-fluorophenyl)cyclobutan-1-amine (39.6 mg, 239 μmol).

LC-MS (method 1): Rₜ=1.18 min; MS (ESIpos): m/z=531 [M+H]⁺

¹H-NMR (400 MHz, DMSO-d6) δ [ppm]: 0.937 (0.83), 0.953 (0.81), 1.759 (0.71), 1.776 (1.37), 1.781 (1.47), 1.798 (1.49), 1.803 (1.67), 1.820 (1.04), 1.842 (0.40), 1.966 (0.92), 1.981 (1.66), 1.997 (1.29), 2.004 (1.73), 2.009 (1.63), 2.020 (1.42), 2.033 (2.06), 2.049 (3.37), 2.065 (3.67), 2.084 (1.94), 2.337 (0.91), 2.349 (1.33), 2.380 (3.18), 2.395 (3.79), 2.418 (2.71), 2.432 (2.17), 2.449 (3.62), 2.518 (10.18), 2.523 (10.77), 2.543 (4.01), 2.674 (1.66), 2.678 (0.75), 2.728 (0.55), 2.889 (0.65), 3.383 (0.81), 3.402 (2.86), 3.427 (7.30), 3.439 (5.27), 3.464 (1.34), 3.511 (1.00), 3.528 (1.81), 3.542 (1.39), 3.551 (1.30), 3.570 (0.66), 4.162 (4.49), 4.180 (8.11), 4.198 (4.30), 6.374 (16.00), 6.510 (12.97), 6.763 (7.58), 6.952 (1.39), 6.956 (1.48), 6.959 (1.70), 6.962 (1.70), 6.975 (2.89), 6.978 (2.66), 6.982 (3.15), 6.995 (1.60), 6.997 (1.62), 7.001 (1.84), 7.004 (1.74), 7.164 (2.38), 7.169 (2.91), 7.174 (2.50), 7.192 (2.36), 7.196 (3.04), 7.202 (2.43), 7.259 (3.03), 7.279 (5.76), 7.300 (3.36), 7.315 (3.48), 7.320 (4.42), 7.335 (4.34), 7.339 (1.80), 7.355 (1.66), 7.731 (4.97), 7.736 (6.64), 7.740 (4.59), 8.221 (0.44), 8.227 (0.47), 8.310 (11.25), 8.315 (11.23).

### Example 322

### 2′-[6-amino-5-(difluoromethoxy)pyridin-3-yl]-N-[1-(propan-2-yl)cyclobutyl]-5′,6′-dihydrospiro[azetidine-3,4′-pyrrolo[1,2-b]pyrazole]-1-carboxamide

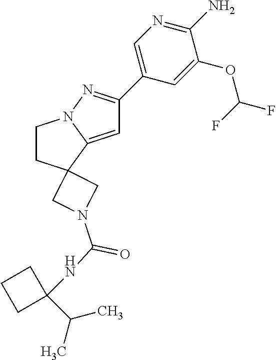 [SMILES: CC(C)C1(NC(=O)N2CC3(CCn4nc(-c5cnc(N)c(OC(F)F)c5)cc43)C2)CCC1]

Example 322 was prepared in analogy to Example 31 using 3-(difluoromethoxy)-5-(5′,6′-dihydrospiro[azetidine-3,4′-pyrrolo[1,2-b]pyrazol]-2′-yl)pyridin-2-amine-hydrochloride salt (75.0 mg, 218 μmol) and 1-(propan-2-yl)cyclobutan-1-amine (29.6 mg, 262 μmol).

LC-MS (method 1): Rₜ=1.06 min; MS (ESIneg): m/z=445 [M−H]⁻

¹H-NMR (400 MHz, DMSO-d6) δ [ppm]: 0.843 (15.63), 0.860 (16.00), 1.066 (0.63), 1.363 (0.59), 1.587 (0.67), 1.611 (0.76), 1.630 (0.49), 1.805 (0.73), 1.820 (0.67), 1.830 (0.70), 1.844 (0.56), 2.014 (0.62), 2.045 (1.57), 2.059 (2.02), 2.068 (1.56), 2.076 (1.95), 2.084 (2.17), 2.092 (2.13), 2.109 (2.88), 2.128 (1.60), 2.159 (0.48), 2.523 (2.41), 2.810 (1.99), 2.827 (3.32), 2.845 (2.10), 2.888 (0.46), 3.299 (0.52), 3.965 (14.44), 3.986 (0.75), 4.097 (2.24), 4.114 (3.57), 4.131 (2.14), 5.767 (0.42), 6.159 (5.79), 6.186 (0.50), 6.209 (4.12), 6.490 (0.41), 6.623 (9.10), 7.000 (2.17), 7.184 (4.25), 7.368 (1.87), 7.547 (0.47), 7.565 (0.52), 7.573 (0.44), 7.596 (0.68), 7.615 (0.63), 7.622 (0.83), 7.649 (3.27), 8.243 (4.54), 8.248 (4.56).

### Example 323

### 2′-[6-amino-5-(difluoromethoxy)pyridin-3-yl]-N-[1-(pyridin-4-yl)cyclobutyl]-5′,6′-dihydrospiro[azetidine-3,4′-pyrrolo[1,2-b]pyrazole]-1-carboxamide

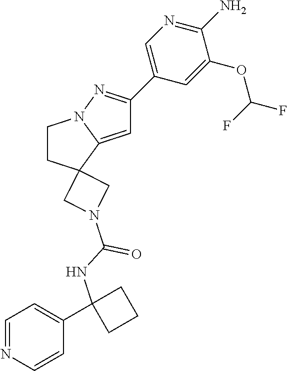 [SMILES: Nc1ncc(-c2cc3n(n2)CCC32CN(C(=O)NC3(c4ccncc4)CCC3)C2)cc1OC(F)F]

Example 323 was prepared in analogy to Example 31 using 3-(difluoromethoxy)-5-(5′,6′-dihydrospiro[azetidine-3,4′-pyrrolo[1,2-b]pyrazol]-2′-yl)pyridin-2-amine-hydrochloride salt (75.0 mg, 218 μmol) and 1-(pyridin-4-yl)cyclobutan-1-amine (38.8 mg, 262 μmol).

LC-MS (method 1): Rₜ=0.86 min; MS (ESIpos): m/z=482 [M+H]⁺

¹H-NMR (400 MHz, DMSO-d6) δ [ppm]: 0.852 (0.58), 0.922 (0.46), 0.937 (3.45), 0.953 (3.33), 1.014 (2.27), 1.032 (2.28), 1.056 (1.05), 1.073 (0.44), 1.232 (2.04), 1.812 (0.50), 1.829 (0.88), 1.835 (0.96), 1.858 (1.10), 1.875 (0.68), 1.985 (0.53), 2.004 (0.91), 2.020 (1.12), 2.033 (0.72), 2.043 (0.90), 2.048 (0.86), 2.059 (0.50), 2.071 (0.46), 2.337 (1.68), 2.357 (0.95), 2.387 (2.17), 2.403 (2.60), 2.409 (2.72), 2.416 (2.78), 2.425 (2.51), 2.434 (2.98), 2.439 (3.08), 2.457 (2.87), 2.518 (16.00), 2.523 (10.44), 2.678 (1.47), 2.829 (2.34), 2.846 (3.80), 2.863 (2.44), 3.821 (1.14), 3.996 (1.75), 4.016 (8.37), 4.023 (8.20), 4.044 (1.76), 4.102 (2.68), 4.120 (4.15), 4.137 (2.67), 6.169 (6.80), 6.421 (1.54), 6.628 (13.09), 6.863 (0.61), 7.003 (2.91), 7.010 (0.45), 7.188 (6.13), 7.194 (0.98), 7.244 (5.61), 7.312 (1.00), 7.315 (0.67), 7.323 (0.63), 7.327 (1.08), 7.340 (0.73), 7.344 (0.46), 7.351 (0.46), 7.355 (0.77), 7.372 (2.64), 7.379 (0.55), 7.393 (7.92), 7.396 (4.89), 7.403 (4.71), 7.408 (8.09), 7.646 (3.71), 7.649 (3.69), 8.215 (0.82), 8.220 (0.80), 8.244 (6.62), 8.248 (6.66), 8.432 (1.13), 8.437 (0.71), 8.444 (0.63), 8.448 (1.02), 8.455 (0.75), 8.460 (0.49), 8.467 (0.50), 8.470 (0.76), 8.501 (8.04), 8.506 (5.02), 8.513 (4.82), 8.517 (7.74).

### Example 324

### 2′-[6-amino-5-(difluoromethoxy)pyridin-3-yl]-N-(bicyclo[2.2.1]heptan-1-yl)-5′,6′-dihydrospiro[azetidine-3,4′-pyrrolo[1,2-b]pyrazole]-1-carboxamide

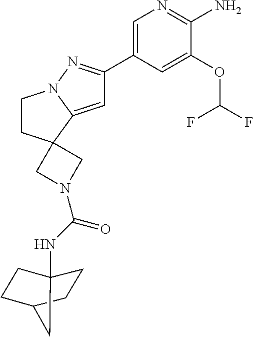 [SMILES: Nc1ncc(-c2cc3n(n2)CCC32CN(C(=O)NC34CCC(CC3)C4)C2)cc1OC(F)F]

Example 324 was prepared in analogy to Example 31 using 3-(difluoromethoxy)-5-(5′,6′-dihydrospiro[azetidine-3,4′-pyrrolo[1,2-b]pyrazol]-2′-yl)pyridin-2-amine-hydrochloride salt (75.0 mg, 218 μmol) and bicyclo[2.2.1]heptan-1-amine-hydrochloride salt (38.7 mg, 262 μmol).

LC-MS (method 1): Rₜ=1.03 min; MS (ESIneg): m/z=443 [M−H]⁻

¹H-NMR (400 MHz, DMSO-d6) δ [ppm]: 0.936 (0.50), 0.953 (0.48), 1.278 (0.91), 1.300 (1.82), 1.319 (1.01), 1.329 (0.97), 1.544 (6.46), 1.588 (1.41), 1.600 (0.94), 1.638 (1.50), 1.664 (1.38), 1.683 (1.54), 1.696 (1.10), 1.716 (1.41), 1.737 (0.50), 2.057 (1.77), 2.084 (3.29), 2.518 (2.03), 2.522 (1.33), 2.802 (2.22), 2.820 (3.58), 2.837 (2.39), 3.950 (0.82), 3.970 (16.00), 3.992 (0.81), 4.093 (2.42), 4.111 (3.75), 4.128 (2.32), 5.770 (0.62), 6.159 (6.18), 6.438 (5.14), 6.623 (12.99), 6.996 (2.73), 7.180 (5.78), 7.364 (2.40), 7.638 (3.32), 7.640 (3.39), 7.642 (3.34), 8.233 (5.98), 8.237 (6.25).

### Example 325

### 2′-[6-amino-5-(difluoromethoxy)pyridin-3-yl]-N-(1-benzylcyclobutyl)-5′,6′-dihydrospiro[azetidine-3,4′-pyrrolo[1,2-b]pyrazole]-1-carboxamide

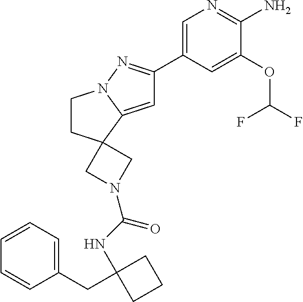 [SMILES: Nc1ncc(-c2cc3n(n2)CCC32CN(C(=O)NC3(Cc4ccccc4)CCC3)C2)cc1OC(F)F]

Example 325 was prepared in analogy to Example 31 using 3-(difluoromethoxy)-5-(5′,6′-dihydrospiro[azetidine-3,4′-pyrrolo[1,2-b]pyrazol]-2′-yl)pyridin-2-amine-hydrochloride salt (75.0 mg, 218 μmol) and 1-benzylcyclobutan-1-amine-hydrochloride salt (51.8 mg, 262 μmol).

LC-MS (method 1): Rt=1.11 min; MS (ESIpos): m/z=495 [M+H]⁺

¹H-NMR (400 MHz, DMSO-d6) δ [ppm]: 1.232 (1.07), 1.753 (1.09), 1.774 (2.36), 1.793 (2.34), 1.809 (1.35), 1.827 (0.56), 2.042 (5.80), 2.059 (8.35), 2.080 (4.23), 2.084 (5.27), 2.332 (1.74), 2.336 (0.79), 2.518 (9.48), 2.522 (5.91), 2.673 (1.83), 2.678 (0.84), 2.817 (2.64), 2.834 (4.28), 2.852 (2.79), 3.079 (9.51), 3.910 (1.26), 3.933 (16.00), 3.954 (1.28), 4.103 (2.87), 4.121 (4.53), 4.138 (2.77), 6.172 (7.77), 6.274 (6.19), 6.617 (15.13), 7.004 (3.18), 7.161 (4.73), 7.178 (5.94), 7.181 (5.47), 7.188 (7.88), 7.202 (1.14), 7.207 (3.38), 7.223 (1.61), 7.226 (2.24), 7.291 (4.90), 7.310 (6.65), 7.328 (2.59), 7.373 (2.84), 7.656 (4.26), 7.658 (4.25), 8.259 (7.24), 8.264 (7.69).

### Example 326

### 2′-[6-amino-5-(difluoromethoxy)pyridin-3-yl]-N-[1-(4-chlorophenyl)cyclobutyl]-5′,6′-dihydrospiro[azetidine-3,4′-pyrrolo[1,2-b]pyrazole]-1-carboxamide

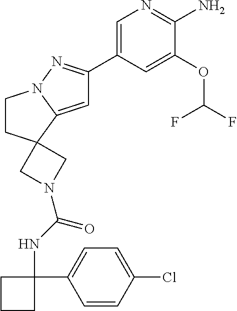 [SMILES: Nc1ncc(-c2cc3n(n2)CCC32CN(C(=O)NC3(c4ccc(Cl)cc4)CCC3)C2)cc1OC(F)F]

Example 326 was prepared in analogy to Example 31 using 3-(difluoromethoxy)-5-(5′,6′-dihydrospiro[azetidine-3,4′-pyrrolo[1,2-b]pyrazol]-2′-yl)pyridin-2-amine-hydrochloride salt (75.0 mg, 218 μmol) and 1-(4-chlorophenyl)cyclobutan-1-amine (47.6 mg, 262 μmol).

LC-MS (method 1): Rₜ=1.14 min; MS (ESIpos): m/z=515 [M+H]⁺

¹H-NMR (400 MHz, DMSO-d6) δ [ppm]: 1.137 (1.41), 1.232 (0.79), 1.743 (0.54), 1.760 (1.02), 1.765 (1.09), 1.782 (1.09), 1.787 (1.24), 1.804 (0.76), 1.978 (0.67), 1.993 (1.22), 2.005 (0.89), 2.016 (1.12), 2.021 (1.10), 2.043 (0.53), 2.084 (7.02), 2.116 (0.62), 2.344 (1.02), 2.366 (1.76), 2.373 (2.33), 2.390 (2.81), 2.396 (2.45), 2.411 (1.80), 2.428 (2.04), 2.444 (2.59), 2.450 (3.03), 2.457 (2.19), 2.467 (3.01), 2.518 (6.34), 2.523 (3.97), 2.812 (3.00), 2.830 (5.02), 2.847 (3.16), 3.964 (2.11), 3.984 (12.26), 3.990 (12.20), 4.011 (2.05), 4.095 (3.32), 4.112 (5.30), 4.130 (3.19), 5.768 (1.01), 6.164 (8.92), 6.612 (16.00), 6.998 (3.48), 7.122 (7.21), 7.183 (7.27), 7.356 (6.83), 7.362 (2.72), 7.367 (3.67), 7.373 (3.60), 7.378 (13.64), 7.384 (2.10), 7.415 (2.04), 7.421 (13.44), 7.426 (3.48), 7.437 (2.51), 7.443 (6.35), 7.643 (5.02), 8.238 (7.68), 8.242 (7.87).

### Example 327

### 2′-[6-amino-5-(difluoromethoxy)pyridin-3-yl]-N-(1-methylcyclobutyl)-5′,6′-dihydrospiro[azetidine-3,4′-pyrrolo[1,2-b]pyrazole]-1-carboxamide

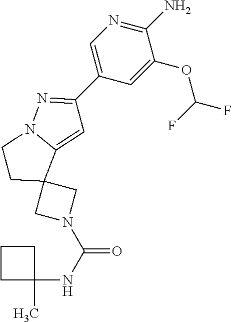 [SMILES: CC1(NC(=O)N2CC3(CCn4nc(-c5cnc(N)c(OC(F)F)c5)cc43)C2)CCC1]

Example 327 was prepared in analogy to Example 31 using 3-(difluoromethoxy)-5-(5′,6′-dihydrospiro[azetidine-3,4′-pyrrolo[1,2-b]pyrazol]-2′-yl)pyridin-2-amine-hydrochloride salt (75.0 mg, 218 μmol) and 1-methylcyclobutan-1-amine-hydrochloride salt (31.8 mg, 262 μmol).

LC-MS (method 1): Rₜ=0.93 min; MS (ESIneg): m/z=417 [M−H]⁻

¹H-NMR (400 MHz, DMSO-d6) δ [ppm]: 1.066 (3.40), 1.297 (6.75), 1.363 (14.56), 1.661 (0.44), 1.680 (1.10), 1.691 (0.79), 1.702 (1.44), 1.708 (1.21), 1.725 (1.87), 1.729 (1.78), 1.735 (1.58), 1.748 (2.62), 1.756 (1.33), 1.762 (0.99), 1.770 (1.50), 1.786 (0.78), 1.794 (0.55), 1.812 (1.09), 1.817 (1.06), 1.822 (1.18), 1.830 (1.29), 1.837 (1.43), 1.841 (1.72), 1.854 (1.51), 1.860 (1.14), 1.873 (0.67), 2.209 (1.01), 2.231 (2.71), 2.254 (2.25), 2.260 (1.89), 2.276 (0.64), 2.283 (0.74), 2.523 (0.66), 2.729 (0.44), 2.805 (2.12), 2.823 (3.54), 2.840 (2.36), 2.888 (0.57), 3.939 (1.33), 3.959 (16.00), 3.980 (0.87), 4.094 (2.31), 4.112 (3.68), 4.129 (2.26), 5.702 (1.07), 5.770 (0.57), 6.159 (5.89), 6.382 (4.70), 6.627 (10.15), 6.998 (2.50), 7.182 (4.60), 7.366 (2.00), 7.644 (3.26), 7.646 (3.19), 8.237 (5.00), 8.242 (5.03).

### Example 328

### (rac)-2-[6-amino-5-(trifluoromethoxy)pyridin-3-yl]-N-[1-(1,3,4-trimethyl-1H-pyrazol-5-yl)ethyl]-6,7-dihydrospiro[pyrazolo[5,1-c][1,4]oxazine-4,3′-pyrrolidine]-1′-carboxamide

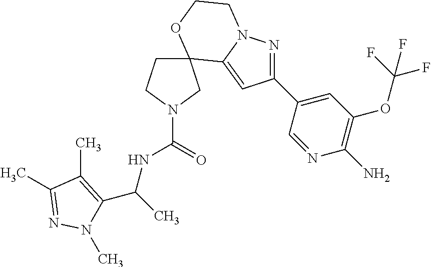 [SMILES: Cc1nn(C)c(C(C)NC(=O)N2CCC3(C2)OCCn2nc(-c4cnc(N)c(OC(F)(F)F)c4)cc23)c1C]

Example 328 was prepared in analogy to Example 31 using (rac)-5-[6,7-dihydrospiro[pyrazolo[5,1-c][1,4]oxazine-4,3′-pyrrolidin]-2-yl]-3-(trifluoromethoxy)pyridin-2-amine (75.0 mg, 211 μmol) and 1-(1,3,4-trimethyl-1H-pyrazol-5-yl)ethan-1-amine (38.8 mg, 253 μmol).

LC-MS (method 1): Rt=0.96 min; MS (ESIpos): m/z=535 [M+H]⁺

¹H-NMR (400 MHz, DMSO-d6) δ [ppm]: 1.389 (3.36), 1.394 (3.61), 1.408 (3.52), 1.413 (3.55), 1.926 (0.44), 1.941 (8.44), 1.947 (8.58), 1.974 (10.98), 2.084 (2.24), 2.202 (0.48), 2.228 (0.64), 2.307 (0.51), 2.323 (0.75), 2.518 (0.92), 2.523 (0.58), 3.359 (0.64), 3.376 (0.52), 3.384 (0.43), 3.397 (1.03), 3.426 (1.03), 3.466 (0.51), 3.495 (0.64), 3.509 (0.42), 3.532 (0.65), 3.606 (0.52), 3.708 (16.00), 3.720 (0.83), 3.751 (0.52), 3.827 (0.61), 3.853 (0.53), 4.043 (0.62), 4.059 (0.78), 4.078 (0.70), 4.096 (0.97), 4.104 (1.23), 4.122 (3.00), 4.897 (0.72), 4.915 (1.09), 4.934 (0.71), 6.444 (0.69), 6.460 (0.68), 6.487 (0.76), 6.503 (0.73), 6.554 (4.17), 6.686 (3.29), 6.699 (2.36), 7.736 (1.50), 7.740 (1.71), 7.744 (1.52), 8.309 (1.84), 8.314 (1.90), 8.318 (2.10), 8.323 (1.93).

### Example 329

### 2-[6-amino-5-(trifluoromethoxy)pyridin-3-yl]-N-[(1R)-2,3-dihydro-1H-inden-1-yl]-6,7-dihydrospiro[pyrazolo[5,1-c][1,4]oxazine-4,3′-pyrrolidine]-1′-carboxamide (Mixture of Stereoisomers)

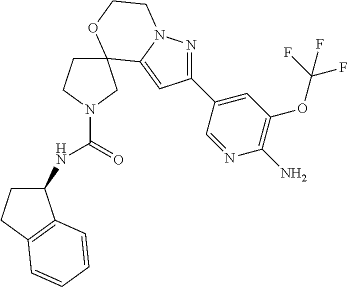 [SMILES: Nc1ncc(-c2cc3n(n2)CCOC32CCN(C(=O)N[C@@H]3CCc4ccccc43)C2)cc1OC(F)(F)F]

Example 329 was prepared in analogy to Example 31 using (rac)-5-[6,7-dihydrospiro[pyrazolo[5,1-c][1,4]oxazine-4,3′-pyrrolidin]-2-yl]-3-(trifluoromethoxy)pyridin-2-amine (75.0 mg, 211 μmol) and (1R)-2,3-dihydro-1H-inden-1-amine (33.7 mg, 253 μmol).

LC-MS (method 1): Rₜ=1.09 min; MS (ESIpos): m/z=515 [M+H]⁺

¹H-NMR (400 MHz, DMSO-d6) δ [ppm]: 1.066 (1.06), 1.788 (0.40), 1.815 (1.31), 1.837 (1.62), 1.864 (1.41), 1.890 (0.50), 2.074 (0.47), 2.230 (1.29), 2.257 (1.62), 2.280 (0.82), 2.327 (2.89), 2.332 (2.23), 2.340 (2.52), 2.358 (2.50), 2.376 (2.32), 2.395 (1.29), 2.408 (0.72), 2.518 (4.51), 2.523 (3.05), 2.563 (1.42), 2.665 (0.94), 2.669 (1.30), 2.673 (0.89), 2.728 (2.24), 2.744 (1.27), 2.762 (1.77), 2.783 (2.17), 2.805 (1.15), 2.876 (1.69), 2.888 (3.39), 2.909 (1.07), 2.932 (0.93), 3.295 (0.53), 3.368 (1.07), 3.385 (1.32), 3.393 (1.31), 3.411 (2.22), 3.428 (1.34), 3.437 (1.20), 3.454 (0.63), 3.469 (2.47), 3.489 (2.55), 3.498 (2.90), 3.518 (2.76), 3.585 (0.78), 3.606 (1.58), 3.637 (1.57), 3.662 (0.65), 3.813 (1.57), 3.833 (1.84), 3.860 (1.39), 4.052 (1.13), 4.067 (2.03), 4.083 (1.97), 4.101 (2.64), 4.125 (6.55), 4.137 (7.99), 4.150 (2.46), 5.178 (1.06), 5.199 (3.08), 5.219 (3.08), 5.240 (1.10), 6.492 (2.53), 6.498 (2.56), 6.513 (2.52), 6.519 (2.47), 6.556 (12.53), 6.711 (16.00), 7.164 (2.67), 7.169 (3.70), 7.173 (4.76), 7.181 (6.46), 7.186 (5.40), 7.191 (5.99), 7.199 (2.13), 7.213 (4.86), 7.226 (5.85), 7.234 (4.14), 7.748 (6.40), 8.326 (7.87), 8.330 (7.79).

### Example 330

### 2′-[6-amino-5-(difluoromethoxy)pyridin-3-yl]-N-[1-(pyridin-2-yl)cyclobutyl]-5′,6′-dihydrospiro[azetidine-3,4′-pyrrolo[1,2-b]pyrazole]-1-carboxamide

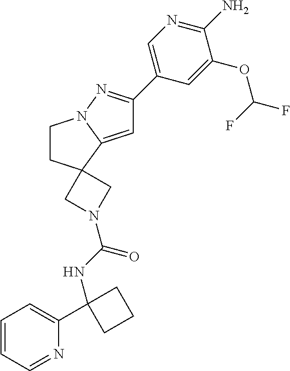 [SMILES: Nc1ncc(-c2cc3n(n2)CCC32CN(C(=O)NC3(c4ccccn4)CCC3)C2)cc1OC(F)F]

Example 330 was prepared in analogy to Example 31 using 3-(difluoromethoxy)-5-(5′,6′-dihydrospiro[azetidine-3,4′-pyrrolo[1,2-b]pyrazol]-2′-yl)pyridin-2-amine-hydrochloride salt (75.0 mg, 218 μmol) and 1-(pyridin-2-yl)cyclobutan-1-amine (38.8 mg, 262 μmol).

LC-MS (method 1): Rt=0.91 min; MS (ESIpos): m/z=482 [M+H]⁺

¹H-NMR (400 MHz, DMSO-d6) δ [ppm]: 1.232 (1.03), 1.812 (0.51), 1.828 (1.17), 1.836 (1.49), 1.839 (1.41), 1.851 (2.96), 1.855 (2.62), 1.863 (2.72), 1.870 (2.46), 1.878 (4.21), 1.886 (2.41), 1.893 (2.82), 1.901 (2.53), 1.908 (1.44), 1.916 (1.38), 1.924 (0.74), 1.931 (0.64), 1.953 (0.80), 1.970 (1.88), 1.977 (2.07), 1.993 (4.14), 2.016 (3.63), 2.019 (3.71), 2.035 (1.95), 2.043 (1.76), 2.060 (0.87), 2.075 (0.48), 2.085 (0.59), 2.224 (3.73), 2.239 (4.14), 2.247 (6.00), 2.254 (6.16), 2.262 (5.04), 2.269 (6.83), 2.277 (4.56), 2.292 (3.41), 2.318 (0.85), 2.337 (1.61), 2.361 (1.94), 2.368 (1.99), 2.377 (1.63), 2.385 (2.22), 2.391 (1.60), 2.408 (1.21), 2.456 (0.53), 2.461 (0.96), 2.518 (9.66), 2.523 (6.86), 2.531 (5.81), 2.537 (8.03), 2.544 (5.94), 2.555 (6.15), 2.561 (7.96), 2.567 (5.03), 2.584 (5.41), 2.601 (1.82), 2.609 (1.94), 2.632 (0.94), 2.845 (2.71), 2.862 (4.39), 2.879 (2.87), 3.295 (0.43), 3.597 (0.59), 4.027 (1.65), 4.048 (11.31), 4.053 (11.15), 4.074 (1.61), 4.113 (3.09), 4.131 (4.69), 4.148 (2.86), 6.170 (7.92), 6.647 (16.00), 6.760 (11.52), 7.007 (3.19), 7.169 (8.98), 7.171 (13.21), 7.181 (7.17), 7.183 (8.15), 7.187 (8.52), 7.190 (13.26), 7.196 (3.48), 7.199 (8.00), 7.202 (7.54), 7.205 (3.06), 7.207 (2.67), 7.211 (2.46), 7.214 (2.40), 7.223 (2.32), 7.226 (2.40), 7.327 (9.18), 7.347 (10.09), 7.375 (6.64), 7.393 (2.65), 7.395 (4.42), 7.398 (2.51), 7.658 (10.31), 7.662 (7.29), 7.677 (8.54), 7.681 (8.58), 7.696 (5.37), 7.701 (5.35), 7.712 (2.29), 7.717 (2.40), 7.731 (2.90), 7.737 (2.82), 7.751 (1.87), 7.756 (1.79), 8.252 (7.42), 8.257 (7.79), 8.513 (7.34), 8.515 (8.71), 8.518 (9.00), 8.520 (7.56), 8.525 (7.52), 8.527 (9.03), 8.530 (8.23), 8.532 (7.15), 8.539 (2.69), 8.541 (2.96), 8.544 (2.82), 8.546 (2.63), 8.551 (2.56), 8.553 (2.87), 8.555 (2.52), 8.558 (2.33).

### Example 331

### 2′-[6-amino-5-(difluoromethoxy)pyridin-3-yl]-N-[1-(3-fluorophenyl)cyclobutyl]-5′,6′-dihydrospiro[azetidine-3,4′-pyrrolo[1,2-b]pyrazole]-1-carboxamide

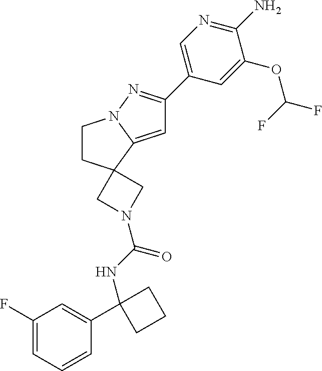 [SMILES: Nc1ncc(-c2cc3n(n2)CCC32CN(C(=O)NC3(c4cccc(F)c4)CCC3)C2)cc1OC(F)F]

Example 331 was prepared in analogy to Example 31 using 3-(difluoromethoxy)-5-(5′,6′-dihydrospiro[azetidine-3,4′-pyrrolo[1,2-b]pyrazol]-2′-yl)pyridin-2-amine-hydrochloride salt (75.0 mg, 218 μmol) and 1-(3-fluorophenyl)cyclobutan-1-amine-hydrochloride salt (52.8 mg, 262 μmol).

LC-MS (method 1): Rₜ=1.09 min; MS (ESIpos): m/z=499 [M+H]⁺

¹H-NMR (400 MHz, DMSO-d6) δ [ppm]: 1.232 (0.49), 1.760 (0.41), 1.765 (0.44), 1.781 (0.89), 1.787 (0.96), 1.798 (0.67), 1.803 (0.96), 1.809 (1.10), 1.819 (0.44), 1.825 (0.68), 1.977 (0.48), 1.983 (0.62), 1.999 (1.09), 2.005 (0.88), 2.016 (0.69), 2.022 (0.99), 2.026 (0.92), 2.038 (0.50), 2.043 (0.47), 2.049 (0.46), 2.318 (0.43), 2.361 (0.80), 2.390 (2.08), 2.407 (2.63), 2.412 (2.35), 2.430 (3.04), 2.447 (2.40), 2.453 (2.66), 2.470 (2.26), 2.518 (6.00), 2.523 (4.12), 2.660 (0.44), 2.819 (2.69), 2.836 (4.43), 2.853 (2.87), 3.979 (2.17), 4.000 (10.04), 4.007 (9.89), 4.028 (2.09), 4.097 (2.98), 4.115 (4.70), 4.131 (2.84), 6.164 (7.89), 6.608 (16.00), 6.992 (0.98), 6.994 (1.14), 6.999 (4.37), 7.014 (1.91), 7.021 (2.13), 7.034 (1.01), 7.036 (1.06), 7.041 (1.20), 7.043 (1.17), 7.129 (6.55), 7.167 (1.58), 7.171 (2.01), 7.173 (2.02), 7.177 (1.90), 7.183 (7.29), 7.194 (1.62), 7.198 (2.05), 7.204 (1.68), 7.253 (2.26), 7.256 (1.65), 7.273 (3.15), 7.336 (1.88), 7.352 (2.10), 7.356 (2.91), 7.367 (3.35), 7.372 (2.97), 7.376 (1.50), 7.392 (1.23), 7.640 (4.25), 7.642 (4.36), 7.644 (4.31), 8.234 (7.76), 8.240 (7.71).

### Example 332

### (rac)-2′-[6-amino-5-(trifluoromethoxy)pyridin-3-yl]-N-[1-(pyridin-2-yl)cyclobutyl]-5′,6′-dihydrospiro[pyrrolidine-3,4′-pyrrolo[1,2-b]pyrazole]-1-carboxamide

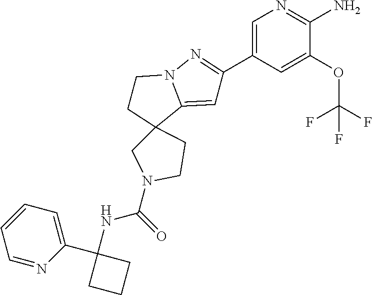 [SMILES: Nc1ncc(-c2cc3n(n2)CCC32CCN(C(=O)NC3(c4ccccn4)CCC3)C2)cc1OC(F)(F)F]

Example 332 was prepared in analogy to Example 31 using (rac)-5-[5′,6′-dihydrospiro[pyrrolidine-3,4′-pyrrolo[1,2-b]pyrazol]-2′-yl]-3-(trifluoromethoxy)pyridin-2-amine-hydrochloride salt (75.0 mg, 200 μmol) and 1-(pyridin-2-yl)cyclobutan-1-amine (35.5 mg, 239 μmol).

LC-MS (method 1): Rₜ=1.00 min; MS (ESIneg): m/z=512 [M−H]⁻

¹H-NMR (400 MHz, DMSO-d6) δ [ppm]: 1.222 (0.66), 1.240 (3.09), 1.255 (2.87), 1.270 (1.78), 1.855 (0.46), 1.862 (0.60), 1.878 (1.14), 1.882 (1.17), 1.889 (1.00), 1.905 (1.62), 1.921 (0.93), 1.927 (0.84), 1.943 (0.58), 1.957 (0.74), 1.964 (0.84), 1.980 (1.58), 1.997 (1.08), 2.004 (1.35), 2.023 (0.79), 2.031 (0.76), 2.047 (0.81), 2.064 (1.60), 2.078 (2.98), 2.096 (3.38), 2.114 (1.38), 2.126 (0.72), 2.337 (0.66), 2.377 (2.31), 2.383 (2.17), 2.389 (2.14), 2.396 (2.37), 2.518 (6.16), 2.523 (4.43), 2.534 (3.71), 2.550 (6.19), 2.553 (6.37), 2.569 (5.52), 2.582 (2.56), 2.594 (2.29), 2.598 (2.16), 2.623 (0.65), 2.679 (0.58), 3.431 (0.94), 3.452 (2.01), 3.478 (8.50), 3.565 (0.89), 3.582 (1.58), 3.596 (1.37), 3.605 (1.26), 3.622 (0.76), 4.178 (4.03), 4.195 (7.04), 4.213 (3.89), 6.427 (16.00), 6.518 (8.39), 6.802 (5.57), 7.178 (1.67), 7.192 (2.11), 7.196 (2.16), 7.209 (1.91), 7.378 (2.60), 7.397 (2.93), 7.671 (1.30), 7.688 (2.19), 7.708 (1.08), 7.750 (4.44), 7.754 (6.14), 7.758 (4.17), 8.330 (10.12), 8.335 (9.89), 8.524 (3.07), 8.526 (3.59), 8.528 (3.68), 8.530 (3.27), 8.536 (3.17), 8.538 (3.68), 8.541 (3.41), 8.542 (2.97).

### Example 333

### 2-[6-amino-5-(difluoromethoxy)pyridin-3-yl]-N-[(1S)-2-methoxy-1-phenylethyl]-6,7-dihydrospiro[pyrazolo[5,1-c][1,4]oxazine-4,3′-pyrrolidine]-1′-carboxamide (Mixture of Stereoisomers)

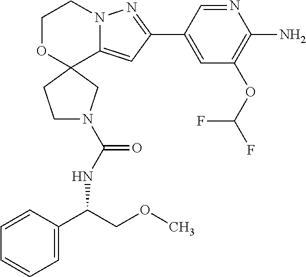 [SMILES: COC[C@@H](NC(=O)N1CCC2(C1)OCCn1nc(-c3cnc(N)c(OC(F)F)c3)cc12)c1ccccc1]

Example 333 was prepared in analogy to Example 31 using (rac)-3-(difluoromethoxy)-5-[6,7-dihydrospiro[pyrazolo[5,1-c][1,4]oxazine-4,3′-pyrrolidin]-2-yl]pyridin-2-amine-hydrochloride salt (75.0 mg, 201 μmol) and (1S)-2-methoxy-1-phenylethan-1-amine (36.4 mg, 241 μmol).

LC-MS (method 1): Rt=0.98 min; MS (ESIpos): m/z=515 [M+H]⁺

1H-NMR (400 MHz, DMSO-d6) δ [ppm]: 2.257 (0.41), 2.323 (0.97), 2.327 (1.39), 2.331 (1.10), 2.518 (7.21), 2.523 (5.04), 2.539 (2.31), 2.665 (0.72), 2.669 (1.00), 2.673 (0.72), 3.257 (16.00), 3.397 (0.59), 3.412 (0.41), 3.428 (0.51), 3.438 (0.62), 3.442 (0.61), 3.453 (1.08), 3.463 (1.15), 3.478 (0.67), 3.490 (0.87), 3.519 (0.56), 3.546 (0.98), 3.567 (1.31), 3.571 (1.02), 3.592 (1.00), 3.620 (0.44), 3.780 (0.51), 3.808 (0.87), 3.837 (0.43), 4.068 (0.57), 4.087 (0.67), 4.104 (1.05), 4.116 (1.52), 4.129 (2.24), 4.942 (0.61), 4.960 (0.62), 6.218 (3.10), 6.496 (0.93), 6.516 (0.88), 6.669 (3.67), 6.996 (0.88), 7.179 (1.74), 7.181 (1.79), 7.203 (0.44), 7.221 (1.24), 7.239 (0.97), 7.289 (1.03), 7.305 (2.21), 7.309 (2.03), 7.323 (1.31), 7.327 (1.36), 7.346 (2.95), 7.364 (2.01), 7.614 (1.83), 8.187 (1.51), 8.192 (2.67), 8.198 (1.67).

### Example 334

### 2′-[6-amino-5-(difluoromethoxy)pyridin-3-yl]-N-[(1R)-1-phenylethyl]-5′,6′-dihydrospiro[pyrrolidine-3,4′-pyrrolo[1,2-b]pyrazole]-1-carboxamide (Mixture of Stereoisomers)

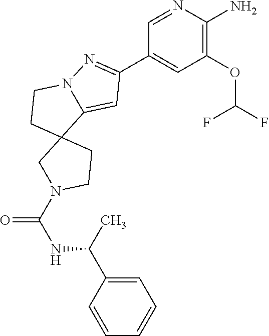 [SMILES: C[C@@H](NC(=O)N1CCC2(CCn3nc(-c4cnc(N)c(OC(F)F)c4)cc32)C1)c1ccccc1]

Example 334 was prepared in analogy to Example 31 using (rac)-3-(difluoromethoxy)-5-[5′,6′-dihydrospiro[pyrrolidine-3,4′-pyrrolo[1,2-b]pyrazol]-2′-yl]pyridin-2-amine-hydrochloride salt (130 mg, 330 μmol) and (1R)-1-phenylethan-1-amine (48.0 mg, 396 μmol).

1H-NMR (400 MHz, DMSO-d6) δ [ppm]: 1.354 (10.16), 1.361 (10.54), 1.372 (10.68), 1.379 (10.21), 2.056 (3.90), 2.066 (3.50), 2.073 (3.76), 2.323 (0.68), 2.327 (0.96), 2.331 (0.73), 2.523 (5.91), 2.529 (8.09), 2.546 (5.26), 2.665 (0.71), 2.669 (1.01), 2.673 (0.77), 3.411 (0.89), 3.428 (2.77), 3.454 (16.00), 3.485 (1.13), 3.514 (0.80), 3.532 (1.59), 3.555 (2.07), 3.570 (1.24), 4.158 (4.72), 4.175 (8.33), 4.193 (4.62), 4.824 (2.04), 4.842 (3.05), 4.861 (2.02), 5.759 (3.69), 6.167 (12.22), 6.346 (6.55), 6.361 (11.17), 6.459 (2.86), 6.464 (3.02), 6.480 (2.95), 6.485 (2.82), 6.999 (5.07), 7.157 (1.10), 7.175 (3.61), 7.183 (11.21), 7.190 (4.10), 7.205 (1.31), 7.208 (2.13), 7.256 (2.72), 7.275 (6.59), 7.294 (6.68), 7.298 (7.20), 7.317 (10.51), 7.339 (7.69), 7.360 (3.43), 7.367 (5.23), 7.617 (7.36), 8.188 (5.77), 8.193 (6.66), 8.195 (7.55), 8.200 (6.27).

### Example 335

### (rac)-2′-[6-amino-5-(difluoromethoxy)pyridin-3-yl]-N-[1-(pyridin-4-yl)cyclobutyl]-5′,6′-dihydrospiro[pyrrolidine-3,4′-pyrrolo[1,2-b]pyrazole]-1-carboxamide

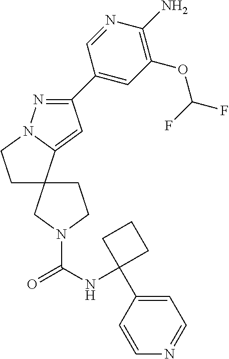 [SMILES: Nc1ncc(-c2cc3n(n2)CCC32CCN(C(=O)NC3(c4ccncc4)CCC3)C2)cc1OC(F)F]

Example 335 was prepared in analogy to Example 31 using (rac)-3-(difluoromethoxy)-5-[5′,6′-dihydrospiro[pyrrolidine-3,4′-pyrrolo[1,2-b]pyrazol]-2′-yl]pyridin-2-amine-hydrochloride salt (130 mg, 330 μmol) and 1-(pyridin-4-yl)cyclobutan-1-amine (58.6 mg, 396 μmol).

LC-MS (method 1): Rt=0.90 min; MS (ESIpos): m/z=496 [M+H]⁺

1H-NMR (400 MHz, DMSO-d6) δ [ppm]: 1.803 (0.74), 1.820 (1.40), 1.826 (1.60), 1.843 (1.60), 1.848 (1.95), 1.866 (1.20), 1.888 (0.46), 1.978 (0.69), 1.985 (0.89), 1.991 (0.89), 2.000 (1.69), 2.008 (1.32), 2.014 (1.89), 2.028 (2.06), 2.043 (2.49), 2.060 (3.32), 2.078 (3.43), 2.084 (9.07), 2.096 (1.49), 2.109 (0.80), 2.261 (0.63), 2.278 (0.77), 2.283 (0.66), 2.300 (0.54), 2.309 (0.63), 2.322 (1.46), 2.326 (2.23), 2.332 (2.00), 2.336 (1.09), 2.347 (1.57), 2.362 (1.77), 2.375 (3.18), 2.391 (3.46), 2.397 (3.06), 2.413 (3.29), 2.442 (2.81), 2.463 (2.52), 2.518 (9.59), 2.523 (5.92), 2.530 (5.90), 2.534 (5.72), 2.550 (3.72), 2.660 (0.54), 2.664 (1.14), 2.669 (1.63), 2.673 (1.20), 2.678 (0.54), 3.415 (2.20), 3.441 (7.44), 3.473 (0.94), 3.525 (0.86), 3.542 (1.57), 4.163 (4.26), 4.180 (8.19), 4.198 (4.18), 5.759 (6.07), 6.174 (12.39), 6.360 (16.00), 6.755 (1.35), 6.871 (7.16), 7.000 (5.12), 7.184 (10.65), 7.304 (2.78), 7.309 (1.72), 7.316 (1.77), 7.319 (2.78), 7.368 (4.55), 7.390 (12.62), 7.394 (7.84), 7.402 (7.58), 7.405 (12.94), 7.618 (6.58), 7.620 (6.41), 8.197 (11.65), 8.202 (10.79), 8.439 (2.98), 8.444 (1.69), 8.451 (1.95), 8.454 (3.23), 8.461 (13.94), 8.464 (8.47), 8.471 (8.19), 8.476 (13.42).

### Example 336

### (rac)-2′-[6-amino-5-(difluoromethoxy)pyridin-3-yl]-N-(1-phenylcyclobutyl)-5′,6′-dihydrospiro[pyrrolidine-3,4′-pyrrolo[1,2-b]pyrazole]-1-carboxamide

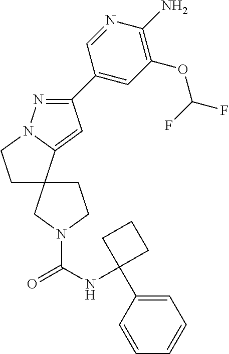 [SMILES: Nc1ncc(-c2cc3n(n2)CCC32CCN(C(=O)NC3(c4ccccc4)CCC3)C2)cc1OC(F)F]

Example 336 was prepared in analogy to Example 31 using (rac)-3-(difluoromethoxy)-5-[5′,6′-dihydrospiro[pyrrolidine-3,4′-pyrrolo[1,2-b]pyrazol]-2′-yl]pyridin-2-amine-hydrochloride salt (130 mg, 330 μmol) and 1-phenylcyclobutan-1-amine-hydrochloride salt (72.7 mg, 396 μmol).

LC-MS (method 1): Rt=1.09 min; MS (ESIpos): m/z=495 [M+H]⁺

1H-NMR (400 MHz, DMSO-d6) δ [ppm]: 1.232 (0.47), 1.764 (1.24), 1.769 (1.32), 1.786 (1.42), 1.791 (1.57), 1.808 (0.99), 1.830 (0.40), 1.964 (0.84), 1.980 (1.54), 2.002 (1.57), 2.007 (1.61), 2.029 (2.24), 2.043 (3.73), 2.062 (3.83), 2.080 (1.57), 2.092 (0.92), 2.356 (1.22), 2.385 (2.73), 2.402 (3.18), 2.423 (2.11), 2.449 (1.94), 2.470 (3.40), 2.518 (16.00), 2.522 (12.72), 2.537 (5.66), 2.888 (0.42), 3.376 (1.04), 3.396 (2.71), 3.422 (6.96), 3.433 (5.39), 3.459 (1.42), 3.502 (0.92), 3.520 (1.69), 3.535 (1.34), 3.561 (0.70), 4.156 (3.88), 4.174 (7.40), 4.191 (3.88), 5.759 (3.70), 6.174 (11.06), 6.329 (14.19), 6.697 (6.61), 6.997 (4.20), 7.134 (1.99), 7.136 (1.22), 7.152 (4.92), 7.170 (3.50), 7.173 (2.24), 7.182 (8.97), 7.261 (5.64), 7.280 (9.47), 7.299 (5.39), 7.366 (3.88), 7.417 (7.85), 7.419 (8.89), 7.438 (7.35), 7.441 (5.89), 7.610 (6.11), 7.612 (5.99), 8.184 (9.49), 8.189 (9.96).

### Example 337

### (rac)-2′-[6-amino-5-(difluoromethoxy)pyridin-3-yl]-N-[2-(pyridin-4-yl)propan-2-yl]-5′,6′-dihydrospiro[pyrrolidine-3,4′-pyrrolo[1,2-b]pyrazole]-1-carboxamide

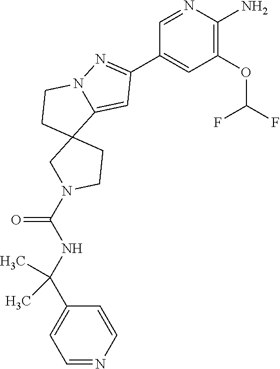 [SMILES: CC(C)(NC(=O)N1CCC2(CCn3nc(-c4cnc(N)c(OC(F)F)c4)cc32)C1)c1ccncc1]

Example 337 was prepared in analogy to Example 31 using (rac)-3-(difluoromethoxy)-5-[5′,6′-dihydrospiro[pyrrolidine-3,4′-pyrrolo[1,2-b]pyrazol]-2′-yl]pyridin-2-amine-hydrochloride salt (130 mg, 330 μmol) and 2-(pyridin-4-yl)propan-2-amine (53.9 mg, 396 μmol).

LC-MS (method 1): Rt=0.88 min; MS (ESIpos): m/z=484 [M+H]⁺

¹H-NMR (400 MHz, DMSO-d₆): δ [ppm]=1.54 (d, 6H), 2.00-2.14 (m, 2H), 2.53-2.58 (m, 3H), 3.39-3.50 (m, 3H), 3.52-3.61 (m, 1H), 4.19 (t, 2H), 6.20 (d, 3H), 6.38 (s, 1H), 6.98-7.39 (m, 1H), 7.29-7.34 (m, 1H), 7.60-7.65 (m, 1H), 8.21 (d, 1H), 8.40-8.45 (m, 2H).

### Example 338

### 2′-[6-amino-5-(difluoromethoxy)pyridin-3-yl]-N-[(1R)-1-(4-fluorophenyl)ethyl]-5′,6′-dihydrospiro[pyrrolidine-3,4′-pyrrolo[1,2-b]pyrazole]-1-carboxamide (Mixture of Stereoisomers)

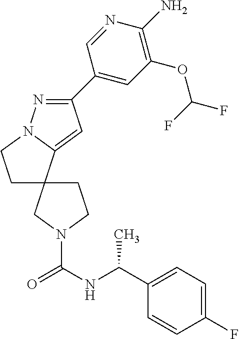 [SMILES: C[C@@H](NC(=O)N1CCC2(CCn3nc(-c4cnc(N)c(OC(F)F)c4)cc32)C1)c1ccc(F)cc1]

Example 338 was prepared in analogy to Example 31 using (rac)-3-(difluoromethoxy)-5-[5′,6′-dihydrospiro[pyrrolidine-3,4′-pyrrolo[1,2-b]pyrazol]-2′-yl]pyridin-2-amine-hydrochloride salt (130 mg, 330 μmol) and (1R)-1-(4-fluorophenyl)ethan-1-amine (55.1 mg, 396 μmol).

LC-MS (method 1): Rt=1.03 min; MS (ESIpos): m/z=487 [M+H]⁺

1H-NMR (400 MHz, DMSO-d6) δ [ppm]: 1.233 (1.08), 1.344 (11.91), 1.351 (12.63), 1.361 (12.63), 1.369 (12.09), 2.038 (2.65), 2.053 (4.81), 2.069 (5.01), 2.083 (2.17), 2.518 (6.64), 2.524 (9.48), 2.527 (9.04), 2.544 (5.87), 3.399 (0.80), 3.418 (2.85), 3.426 (2.73), 3.449 (14.96), 3.475 (1.94), 3.506 (0.94), 3.524 (1.78), 3.532 (1.80), 3.538 (1.58), 3.551 (2.47), 3.558 (1.30), 3.565 (1.66), 3.577 (1.08), 4.157 (5.49), 4.175 (10.29), 4.192 (5.33), 4.820 (2.49), 4.839 (3.71), 4.857 (2.47), 6.166 (15.92), 6.360 (10.33), 6.365 (16.00), 6.470 (3.29), 6.479 (3.41), 6.490 (3.33), 6.499 (3.11), 6.997 (5.97), 7.071 (4.15), 7.076 (1.56), 7.093 (10.11), 7.114 (10.69), 7.130 (1.60), 7.136 (5.23), 7.182 (12.35), 7.338 (4.37), 7.343 (1.98), 7.352 (5.27), 7.361 (8.30), 7.366 (8.42), 7.374 (7.98), 7.383 (4.71), 7.392 (1.64), 7.397 (3.69), 7.620 (9.40), 8.195 (8.32), 8.198 (11.01), 8.202 (8.86).

### Example 339

### (rac)-2′-[6-amino-5-(difluoromethoxy)pyridin-3-yl]-N-[2-(4-fluorophenyl)propan-2-yl]-5′,6′-dihydrospiro[pyrrolidine-3,4′-pyrrolo[1,2-b]pyrazole]-1-carboxamide

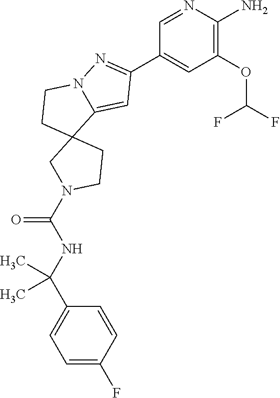 [SMILES: CC(C)(NC(=O)N1CCC2(CCn3nc(-c4cnc(N)c(OC(F)F)c4)cc32)C1)c1ccc(F)cc1]

Example 339 was prepared in analogy to Example 31 using (rac)-3-(difluoromethoxy)-5-[5′,6′-dihydrospiro[pyrrolidine-3,4′-pyrrolo[1,2-b]pyrazol]-2′-yl]pyridin-2-amine-hydrochloride salt (130 mg, 330 μmol) and 2-(4-fluorophenyl)propan-2-amine (60.6 mg, 396 μmol).

LC-MS (method 1): Rt=1.09 min; MS (ESIpos): m/z=501 [M+H]⁺

1H-NMR (400 MHz, DMSO-d6) δ [ppm]: 1.555 (16.00), 2.033 (0.88), 2.050 (1.54), 2.070 (1.66), 2.084 (3.71), 2.523 (3.12), 2.526 (3.43), 2.546 (1.99), 3.410 (0.86), 3.417 (1.04), 3.445 (4.48), 3.543 (0.87), 3.557 (0.70), 3.565 (0.64), 4.164 (2.09), 4.182 (3.83), 4.199 (2.07), 6.069 (4.13), 6.175 (6.04), 6.364 (8.90), 7.000 (2.11), 7.027 (2.66), 7.032 (1.04), 7.050 (5.65), 7.067 (1.01), 7.072 (3.11), 7.184 (4.31), 7.353 (2.84), 7.358 (1.44), 7.367 (4.54), 7.375 (3.15), 7.384 (1.19), 7.389 (2.60), 7.620 (3.25), 8.202 (4.68), 8.207 (4.76).

### Example 340

### 2′-[6-amino-5-(difluoromethoxy)pyridin-3-yl]-N-[(1R)-1-(5-fluoropyridin-3-yl)ethyl]-5′,6′-dihydrospiro[pyrrolidine-3,4′-pyrrolo[1,2-b]pyrazole]-1-carboxamide (Mixture of Stereoisomers)

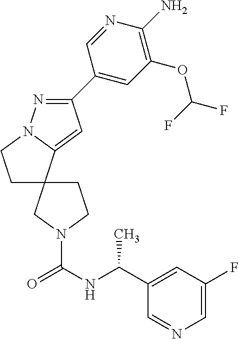 [SMILES: C[C@@H](NC(=O)N1CCC2(CCn3nc(-c4cnc(N)c(OC(F)F)c4)cc32)C1)c1cncc(F)c1]

Example 340 was prepared in analogy to Example 31 using (rac)-3-(difluoromethoxy)-5-[5′,6′-dihydrospiro[pyrrolidine-3,4′-pyrrolo[1,2-b]pyrazol]-2′-yl]pyridin-2-amine-hydrochloride salt (130 mg, 330 μmol) and (1R)-1-(5-fluoropyridin-3-yl)ethan-1-amine (55.5 mg, 396 μmol).

LC-MS (method 1): Rt=0.89 min; MS (ESIpos): m/z=488 [M+H]⁺

¹H-NMR (400 MHz, DMSO-d₆): δ [ppm]=1.41 (dd, 3H), 2.00-2.14 (m, 2H), 2.52-2.58 (m, 2H), 3.39-3.64 (m, 4H), 4.18 (t, 2H), 4.87-4.97 (m, 1H), 6.16 (s, 2H), 6.33-6.39 (m, 1H), 6.60 (dd, 1H), 6.98-7.39 (m, 1H), 7.62 (s, 1H), 7.68 (ddt, 1H), 8.16-8.22 (m, 1H), 8.38-8.48 (m, 2H).

### Example 341

### 2′-{6-amino-5-[(1R)-1-(pyridin-2-yl)ethoxy]pyridin-3-yl}-N-[(1R)-1-phenylethyl]-5′,6′-dihydrospiro[pyrrolidine-3,4′-pyrrolo[1,2-b]pyrazole]-1-carboxamide (Mixture of Stereoisomers)

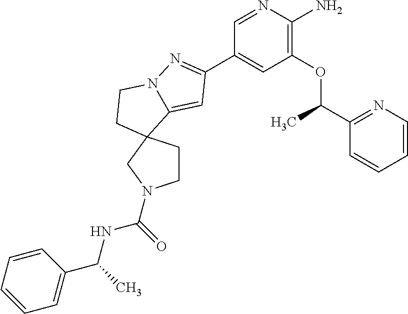 [SMILES: C[C@@H](NC(=O)N1CCC2(CCn3nc(-c4cnc(N)c(O[C@H](C)c5ccccn5)c4)cc32)C1)c1ccccc1]

Example 341 was prepared in analogy to Example 31 using 5-[5′,6′-dihydrospiro[pyrrolidine-3,4′-pyrrolo[1,2-b]pyrazol]-2′-yl]-3-[(1R)-1-(pyridin-2-yl)ethoxy]pyridin-2-amine-hydrochloride salt (50.0 mg, 121 μmol) and (1R)-1-phenylethan-1-amine (17.6 mg, 145 μmol).

LC-MS (method 1): Rₜ=0.99 min; MS (ESIpos): m/z=524 [M+H]⁺

¹H-NMR (400 MHz, DMSO-d6) δ [ppm]: 0.851 (0.63), 1.233 (2.25), 1.284 (0.95), 1.301 (0.85), 1.351 (8.50), 1.356 (8.28), 1.369 (8.85), 1.374 (8.05), 1.501 (0.57), 1.519 (0.64), 1.610 (15.95), 1.626 (16.00), 2.022 (2.91), 2.037 (3.75), 2.055 (1.62), 2.067 (0.81), 2.083 (0.43), 2.472 (3.83), 2.522 (3.96), 3.383 (0.95), 3.393 (1.36), 3.401 (1.68), 3.419 (6.31), 3.426 (12.08), 3.452 (1.43), 3.490 (0.62), 3.509 (1.30), 3.532 (1.58), 3.546 (0.98), 3.571 (0.43), 4.109 (3.48), 4.125 (6.34), 4.143 (3.45), 4.818 (1.10), 4.824 (1.27), 4.837 (1.63), 4.843 (1.75), 4.855 (1.23), 4.862 (1.16), 5.487 (1.03), 5.503 (3.73), 5.520 (3.74), 5.536 (1.05), 5.901 (8.53), 6.200 (5.92), 6.210 (9.28), 6.446 (2.23), 6.452 (2.20), 6.466 (2.23), 6.473 (2.02), 7.151 (0.81), 7.170 (2.86), 7.188 (3.86), 7.202 (4.87), 7.206 (7.79), 7.248 (2.18), 7.268 (5.80), 7.275 (4.31), 7.279 (3.86), 7.281 (4.20), 7.286 (6.34), 7.291 (4.53), 7.296 (6.63), 7.303 (3.40), 7.310 (6.17), 7.314 (5.96), 7.327 (3.02), 7.338 (5.34), 7.356 (2.63), 7.384 (0.45), 7.490 (4.17), 7.510 (4.72), 7.762 (1.58), 7.766 (2.26), 7.769 (1.58), 7.781 (2.65), 7.785 (3.79), 7.788 (2.52), 7.800 (1.36), 7.804 (1.81), 7.808 (1.24), 7.850 (4.47), 7.854 (4.74), 7.859 (5.04), 7.864 (4.65), 8.539 (3.39), 8.544 (2.27), 8.546 (2.35), 8.549 (3.36), 8.552 (3.36).

### Example 342

### 2′-{6-amino-5-[(1R)-1-(pyridin-2-yl)ethoxy]pyridin-3-yl}-N-[2-(pyridin-4-yl)propan-2-yl]-5′,6′-dihydrospiro[pyrrolidine-3,4′-pyrrolo[1,2-b]pyrazole]-1-carboxamide (Mixture of Stereoisomers)

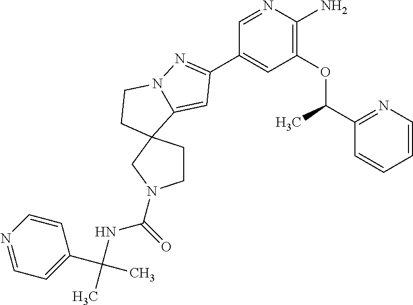 [SMILES: C[C@@H](Oc1cc(-c2cc3n(n2)CCC32CCN(C(=O)NC(C)(C)c3ccncc3)C2)cnc1N)c1ccccn1]

Example 342 was prepared in analogy to Example 31 using 5-[5′,6′-dihydrospiro[pyrrolidine-3,4′-pyrrolo[1,2-b]pyrazol]-2′-yl]-3-[(1R)-1-(pyridin-2-yl)ethoxy]pyridin-2-amine-hydrochloride salt (50.0 mg, 121 μmol) and 2-(pyridin-4-yl)propan-2-amine (19.8 mg, 145 μmol).

LC-MS (method 1): Rₜ=0.87 min; MS (ESIpos): m/z=539 [M+H]⁺

¹H-NMR (400 MHz, DMSO-d6) δ [ppm]: 1.020 (0.68), 1.036 (0.73), 1.448 (0.44), 1.516 (1.99), 1.527 (7.04), 1.534 (16.00), 1.616 (6.47), 1.632 (6.52), 2.005 (0.49), 2.029 (0.92), 2.045 (1.00), 2.053 (0.84), 2.074 (0.55), 2.518 (3.37), 2.522 (1.72), 3.399 (0.91), 3.424 (2.69), 3.529 (0.63), 4.119 (1.80), 4.137 (3.26), 4.154 (1.76), 5.491 (0.47), 5.507 (1.73), 5.523 (1.74), 5.539 (0.47), 5.914 (4.77), 6.207 (3.24), 6.231 (6.45), 7.211 (1.95), 7.214 (3.34), 7.219 (1.89), 7.271 (0.78), 7.274 (1.41), 7.276 (0.89), 7.283 (0.84), 7.286 (1.52), 7.289 (1.47), 7.292 (1.57), 7.295 (1.00), 7.302 (3.70), 7.307 (5.03), 7.310 (2.34), 7.314 (2.17), 7.317 (4.40), 7.322 (3.17), 7.495 (1.92), 7.497 (1.92), 7.514 (2.18), 7.517 (2.14), 7.766 (0.96), 7.770 (1.03), 7.785 (1.68), 7.789 (1.71), 7.805 (0.78), 7.808 (0.80), 7.872 (2.71), 7.875 (3.57), 7.879 (2.70), 8.412 (3.17), 8.416 (2.08), 8.420 (3.56), 8.423 (3.43), 8.427 (3.41), 8.430 (1.98), 8.435 (3.09), 8.541 (1.51), 8.543 (1.74), 8.545 (1.76), 8.548 (1.58), 8.553 (1.55), 8.555 (1.75), 8.558 (1.65), 8.560 (1.44).

### Example 343

### 2′-{6-amino-5-[(1S)-1-(pyridin-2-yl)ethoxy]pyridin-3-yl}-N-[(1R)-1-phenylethyl]-5′,6′-dihydrospiro[pyrrolidine-3,4′-pyrrolo[1,2-b]pyrazole]-1-carboxamide (Mixture of Stereoisomers)

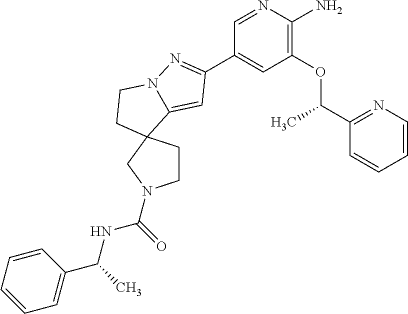 [SMILES: C[C@H](Oc1cc(-c2cc3n(n2)CCC32CCN(C(=O)N[C@H](C)c3ccccc3)C2)cnc1N)c1ccccn1]

Example 343 was prepared in analogy to Example 31 using 5-[5′,6′-dihydrospiro[pyrrolidine-3,4′-pyrrolo[1,2-b]pyrazol]-2′-yl]-3-[(1S)-1-(pyridin-2-yl)ethoxy]pyridin-2-amine-hydrochloride salt (65.0 mg, 157 μmol) and (1R)-1-phenylethan-1-amine (22.9 mg, 189 μmol).

LC-MS (method 1): Rₜ=0.99 min; MS (ESIpos): m/z=524 [M+H]⁺

¹H-NMR (400 MHz, DMSO-d6) δ [ppm]: 0.852 (0.57), 0.943 (0.42), 1.232 (2.20), 1.256 (0.57), 1.284 (1.07), 1.301 (1.05), 1.350 (8.42), 1.361 (9.11), 1.367 (9.37), 1.379 (8.14), 1.611 (15.80), 1.627 (16.00), 1.754 (0.76), 1.988 (0.52), 2.002 (1.29), 2.014 (2.48), 2.030 (3.55), 2.045 (2.84), 2.062 (1.24), 2.076 (0.61), 2.473 (3.58), 2.518 (2.11), 2.523 (1.43), 3.167 (1.88), 3.390 (1.50), 3.400 (1.63), 3.409 (2.64), 3.422 (10.44), 3.433 (7.01), 3.452 (1.39), 3.457 (1.17), 3.490 (0.72), 3.507 (1.35), 3.516 (1.28), 3.521 (1.14), 3.534 (1.69), 3.542 (0.93), 3.549 (1.19), 3.556 (0.77), 3.575 (0.47), 4.109 (3.93), 4.126 (7.26), 4.143 (3.81), 4.655 (0.92), 4.805 (0.49), 4.823 (1.99), 4.841 (2.91), 4.860 (1.97), 4.877 (0.48), 5.491 (1.02), 5.506 (3.63), 5.523 (3.60), 5.539 (1.03), 5.902 (10.00), 6.202 (6.84), 6.214 (10.81), 6.446 (2.35), 6.455 (2.35), 6.466 (2.31), 6.476 (2.11), 7.156 (0.50), 7.160 (0.96), 7.164 (1.00), 7.167 (1.12), 7.171 (1.14), 7.178 (2.54), 7.185 (2.79), 7.191 (1.76), 7.195 (2.13), 7.199 (2.36), 7.207 (8.57), 7.212 (7.93), 7.261 (2.95), 7.263 (2.49), 7.275 (7.06), 7.278 (7.62), 7.283 (5.01), 7.286 (2.93), 7.295 (9.05), 7.302 (2.42), 7.305 (2.39), 7.315 (6.12), 7.318 (5.75), 7.336 (7.98), 7.354 (2.85), 7.358 (2.00), 7.492 (3.60), 7.495 (3.63), 7.512 (4.05), 7.514 (4.09), 7.758 (1.43), 7.763 (2.47), 7.768 (1.63), 7.777 (2.41), 7.782 (4.18), 7.787 (2.59), 7.796 (1.24), 7.801 (1.97), 7.806 (1.26), 7.856 (5.92), 7.860 (7.99), 7.863 (6.19), 8.067 (2.23), 8.532 (1.72), 8.534 (2.08), 8.538 (3.44), 8.544 (3.49), 8.550 (3.37), 8.554 (2.12), 8.556 (1.81).

### Example 344

### 2′-{6-amino-5-[(1S)-1-(pyridin-2-yl)ethoxy]pyridin-3-yl}-N-[2-(pyridin-4-yl)propan-2-yl]-5′,6′-dihydrospiro[pyrrolidine-3,4′-pyrrolo[1,2-b]pyrazole]-1-carboxamide (Mixture of Stereoisomers)

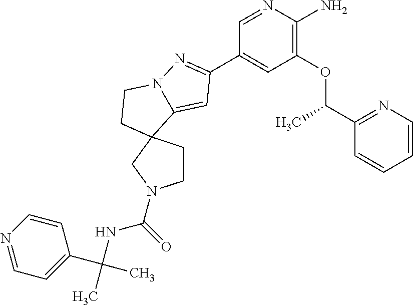 [SMILES: C[C@H](Oc1cc(-c2cc3n(n2)CCC32CCN(C(=O)NC(C)(C)c3ccncc3)C2)cnc1N)c1ccccn1]

Example 344 was prepared in analogy to Example 31 using 5-[5′,6′-dihydrospiro[pyrrolidine-3,4′-pyrrolo[1,2-b]pyrazol]-2′-yl]-3-[(1S)-1-(pyridin-2-yl)ethoxy]pyridin-2-amine-hydrochloride salt (65.0 mg, 157 μmol) and 2-(pyridin-4-yl)propan-2-amine (25.7 mg, 189 μmol).

LC-MS (method 1): Rₜ=0.87 min; MS (ESIpos): m/z=539 [M+H]⁺

¹H-NMR (400 MHz, DMSO-d6) δ [ppm]: 1.233 (0.64), 1.527 (7.26), 1.534 (16.00), 1.617 (6.59), 1.632 (6.59), 2.006 (0.55), 2.028 (0.97), 2.045 (1.02), 2.054 (0.83), 2.062 (0.53), 2.072 (0.46), 2.482 (1.79), 2.518 (2.14), 3.162 (11.10), 3.171 (11.24), 3.399 (0.94), 3.425 (2.72), 3.530 (0.64), 4.104 (1.82), 4.119 (3.23), 4.137 (3.63), 4.155 (1.84), 5.492 (0.48), 5.508 (1.76), 5.525 (1.76), 5.541 (0.48), 5.915 (4.86), 6.209 (3.28), 6.232 (6.55), 7.212 (1.99), 7.216 (3.35), 7.221 (1.95), 7.270 (0.74), 7.273 (1.30), 7.276 (0.81), 7.282 (0.81), 7.285 (1.43), 7.289 (1.45), 7.291 (1.54), 7.295 (0.92), 7.303 (4.17), 7.307 (5.04), 7.311 (2.24), 7.314 (2.18), 7.319 (4.34), 7.322 (3.11), 7.495 (1.95), 7.497 (1.92), 7.515 (2.23), 7.518 (2.15), 7.766 (0.98), 7.769 (1.00), 7.785 (1.70), 7.789 (1.71), 7.804 (0.79), 7.808 (0.78), 7.874 (2.78), 7.877 (3.70), 7.882 (2.66), 8.413 (3.06), 8.416 (1.99), 8.420 (3.46), 8.424 (3.30), 8.428 (3.26), 8.432 (1.90), 8.435 (2.92), 8.541 (1.53), 8.544 (1.76), 8.546 (1.83), 8.548 (1.56), 8.553 (1.55), 8.556 (1.78), 8.558 (1.69), 8.560 (1.41).

### Example 345

### 2′-{6-amino-5-[(trifluoromethyl)sulfanyl]pyridin-3-yl}-N-[(1R)-1-(3-fluorophenyl)ethyl]-5′,6′-dihydrospiro[pyrrolidine-3,4′-pyrrolo[1,2-b]pyrazole]-1-carboxamide (Mixture of Stereoisomers)

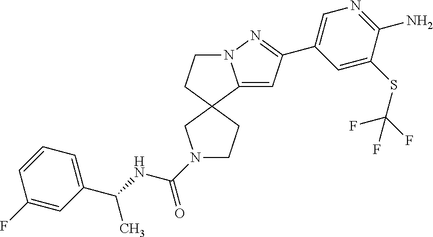 [SMILES: C[C@@H](NC(=O)N1CCC2(CCn3nc(-c4cnc(N)c(SC(F)(F)F)c4)cc32)C1)c1cccc(F)c1]

Example 345 was prepared in analogy to Example 31 using (rac)-5-[5′,6′-dihydrospiro[pyrrolidine-3,4′-pyrrolo[1,2-b]pyrazol]-2′-yl]-3-[(trifluoromethyl)sulfanyl]pyridin-2-amine-hydrochloride salt (20.0 mg, 51.0 μmol) and (1R)-1-(3-fluorophenyl)ethan-1-amine (8.52 mg, 61.2 μmol).

LC-MS (method 1): Rt=1.15 min; MS (ESIpos): m/z=521 [M+H]⁺

¹H-NMR (400 MHz, DMSO-d6) δ [ppm]: 1.021 (5.37), 1.026 (4.82), 1.039 (6.90), 1.057 (1.83), 1.231 (0.72), 1.282 (1.39), 1.299 (1.55), 1.344 (5.57), 1.351 (14.20), 1.357 (15.16), 1.369 (14.52), 1.375 (14.05), 1.496 (0.76), 1.513 (0.76), 2.050 (3.51), 2.068 (6.04), 2.074 (6.57), 2.084 (7.45), 2.518 (14.51), 2.536 (11.93), 2.554 (7.38), 3.113 (0.43), 3.131 (1.28), 3.148 (1.22), 3.283 (0.44), 3.353 (3.16), 3.426 (3.97), 3.452 (10.23), 3.458 (14.36), 3.471 (6.99), 3.497 (1.97), 3.513 (1.17), 3.530 (2.10), 3.554 (2.01), 3.571 (2.31), 3.585 (1.35), 3.596 (1.29), 3.612 (0.68), 4.167 (6.50), 4.184 (11.23), 4.202 (6.32), 4.263 (0.50), 4.280 (0.69), 4.297 (0.53), 4.833 (2.41), 4.850 (3.64), 4.867 (2.46), 5.454 (0.44), 5.768 (0.47), 6.396 (7.95), 6.414 (15.55), 6.506 (3.49), 6.517 (3.79), 6.526 (3.70), 6.537 (3.41), 6.743 (16.00), 6.972 (1.40), 6.978 (1.62), 6.992 (4.01), 7.014 (4.01), 7.028 (1.57), 7.033 (1.94), 7.037 (1.63), 7.102 (0.84), 7.134 (2.87), 7.140 (3.16), 7.148 (3.53), 7.168 (8.65), 7.190 (6.29), 7.289 (2.00), 7.303 (2.37), 7.309 (3.51), 7.318 (1.64), 7.324 (3.80), 7.331 (3.89), 7.347 (3.10), 7.366 (1.17), 7.529 (0.42), 8.036 (5.69), 8.041 (10.57), 8.047 (6.25), 8.512 (8.02), 8.517 (13.46), 8.522 (8.70).

### Example 346

### 2′-[6-amino-5-(trifluoromethyl)pyridin-3-yl]-N-[(3R)-2,3-dihydro-1-benzofuran-3-yl]-5′,6′-dihydrospiro[pyrrolidine-3,4′-pyrrolo[1,2-b]pyrazole]-1-carboxamide (Mixture of Stereoisomers)

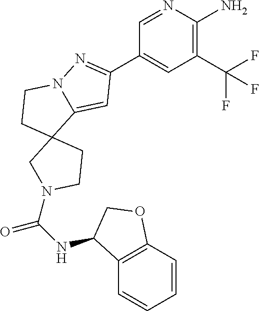 [SMILES: Nc1ncc(-c2cc3n(n2)CCC32CCN(C(=O)N[C@H]3COc4ccccc43)C2)cc1C(F)(F)F]

Example 346 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine-hydrochloride salt (75.0 mg, 208 μmol) and 1): (3R)-2,3-dihydro-1-benzofuran-3-amine-hydrochloride salt (42.9 mg, 250 μmol).

LC-MS (method 1): Rt=1.05 min; MS (ESIpos): m/z=485 [M+H]⁺

1H-NMR (400 MHz, DMSO-d6): δ [ppm]=2.00-2.11 (m, 2H), 2.52-2.56 (m, 2H), 3.39-3.61 (m, 4H), 4.17 (t, 2H), 4.24 (dt, 1H), 4.66 (td, 1H), 5.43-5.50 (m, 1H), 6.46 and 6.48 (2s, 1H), 6.54 (s, 2H), 6.76 (dd, 1H), 6.80 (d, 1H), 6.83-6.90 (m, 1H), 7.18 (tdd, 1H), 7.30-7.35 (m, 1H), 8.01 (s, 1H), 8.59 (d, 1H).

### Example 347

### 2′-{6-amino-5-[(1R)-1-(pyridin-3-yl)ethoxy]pyridin-3-yl}-N-[2-(pyridin-4-yl)propan-2-yl]-5′,6′-dihydrospiro[pyrrolidine-3,4′-pyrrolo[1,2-b]pyrazole]-1-carboxamide (Mixture of Stereoisomers)

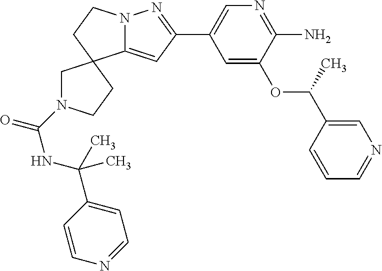 [SMILES: C[C@@H](Oc1cc(-c2cc3n(n2)CCC32CCN(C(=O)NC(C)(C)c3ccncc3)C2)cnc1N)c1cccnc1]

Example 347 was prepared in analogy to Example 31 using 5-[5′,6′-dihydrospiro[pyrrolidine-3,4′-pyrrolo[1,2-b]pyrazol]-2′-yl]-3-[(1R)-1-(pyridin-3-yl)ethoxy]pyridin-2-amine-hydrochloride salt (82.7 mg, 79% purity, 157 μmol) and 2-(pyridin-4-yl)propan-2-amine (25.7 mg, 189 μmol).

LC-MS (method 1): Rt=0.87 min; MS (ESIpos): m/z=539 [M+H]⁺

1H-NMR (400 MHz, DMSO-d6): 5 [ppm]=1.52 and 1.54 (2s, 6H), 1.60 (d, 3H), 1.98-2.12 (m, 2H), 3.38-3.48 (m, 3H), 3.51-3.59 (m, 1H), 4.15 (t, 2H), 5.67-5.74 (m, 1H), 5.91 (s, 2H), 6.21 and 6.22 (2s, 1H), 6.28 (s, 1H), 7.29-7.34 (m, 3H), 7.38 (dd, 1H), 7.88 (d, 1H), 7.92 (dt, 1H), 8.40-8.44 (m, 2H), 8.47 (dd, 1H), 8.71 (d, 1H).

### Example 348

### 2′-[6-amino-5-(trifluoromethyl)pyridin-3-yl]-N-[1-(1-methyl-1H-imidazol-5-yl)ethyl]-5′,6′-dihydrospiro[pyrrolidine-3,4′-pyrrolo[1,2-b]pyrazole]-1-carboxamide (Diastereomeric Mixture)

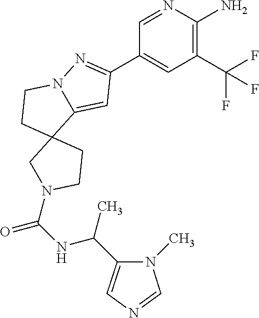 [SMILES: CC(NC(=O)N1CCC2(CCn3nc(-c4cnc(N)c(C(F)(F)F)c4)cc32)C1)c1cncn1C]

Example 348 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine-hydrochloride salt (70.0 mg, 195 μmol) and 1-(1-methyl-1H-imidazol-5-yl)ethan-1-amine-hydrochloride salt (46.3 mg, 233 μmol).

LC-MS (method 1): Rt=0.84 min; MS (ESIpos): m/z=475 [M+H]⁺

1H-NMR (400 MHz, DMSO-d6): δ [ppm]=1.41 and 1.43 (2d, 3H), 1.99-2.12 (m, 2H), 3.39-3.61 (m, 7H), 4.17 (t, 2H), 4.93 and 4.96 (2q, 1H), 6.34 an 6.36 (2br d, 1H), 6.44 and 6.45 (2s, 1H), 6.54 (s, 2H), 6.79 and 6.80 (2s, 1H), 7.49 and 7.50 (2br s, 1H), 8.01 (d, 1H), 8.58 (d, 1H).

### Example 349

### 2′-{6-amino-5-[(1R)-1-(pyridin-3-yl)ethoxy]pyridin-3-yl}-N-cyclobutyl-5′,6′-dihydrospiro[pyrrolidine-3,4′-pyrrolo[1,2-b]pyrazole]-1-carboxamide (Mixture of Stereoisomers)

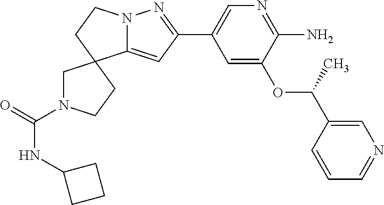 [SMILES: C[C@@H](Oc1cc(-c2cc3n(n2)CCC32CCN(C(=O)NC3CCC3)C2)cnc1N)c1cccnc1]

Example 349 was prepared in analogy to Example 31 using 5-[5′,6′-dihydrospiro[pyrrolidine-3,4′-pyrrolo[1,2-b]pyrazol]-2′-yl]-3-[(1R)-1-(pyridin-3-yl)ethoxy]pyridin-2-amine-hydrochloride salt (82.7 mg, 79% purity, 157 μmol) and cyclobutanamine (13.4 mg, 189 μmol).

LC-MS (method 1): Rt=0.87 min; MS (ESIpos): m/z=474 [M+H]⁺

1H-NMR (400 MHz, DMSO-d6): δ [ppm]=1.46-1.62 (m, 5H), 1.86-2.16 (m, 6H), 3.36-3.43 (m, 3H), 3.44-3.53 (m, 1H), 4.06-4.19 (m, 3H), 5.71 (q, 1H), 5.89 (s, 2H), 6.27 (2s, 1H), 6.31 (d, 1H), 7.32 (s, 1H), 7.37 (dd, 1H), 7.87 (t, 1H), 7.88-7.94 (m, 1H), 8.47 (dd, 1H), 8.70 (2s, 1H).

### Example 350

### 2′-{6-amino-5-[(1R)-1-(pyridin-3-yl)ethoxy]pyridin-3-yl}-N-[2-(1-methyl-1H-1,2,3-triazol-4-yl)propan-2-yl]-5′,6′-dihydrospiro[pyrrolidine-3,4′-pyrrolo[1,2-b]pyrazole]-1-carboxamide (Mixture of Stereoisomers)

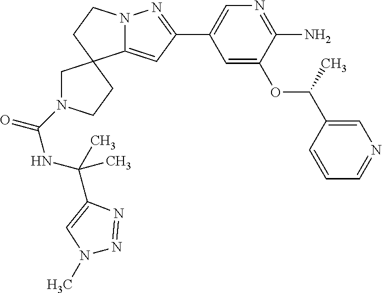 [SMILES: C[C@@H](Oc1cc(-c2cc3n(n2)CCC32CCN(C(=O)NC(C)(C)c3cn(C)nn3)C2)cnc1N)c1cccnc1]

Example 350 was prepared in analogy to Example 31 using 5-[5′,6′-dihydrospiro[pyrrolidine-3,4′-pyrrolo[1,2-b]pyrazol]-2′-yl]-3-[(1R)-1-(pyridin-3-yl)ethoxy]pyridin-2-amine-hydrochloride salt (82.7 mg, 79% purity, 157 μmol) and 2-(1-methyl-1H-1,2,3-triazol-4-yl)propan-2-amine-hydrochloride salt (40.2 mg, 189 μmol).

LC-MS (method 1): Rt=0.81 min; MS (ESIpos): m/z=543 [M+H]⁺

¹H-NMR (400 MHz, DMSO-d₆): δ [ppm]=1.56-1.66 (m, 9H), 1.96-2.10 (m, 2H), 3.37-3.40 (m, 3H), 3.46-3.57 (m, 1H), 3.97 (2s, 3H), 4.14 (br t, 2H), 5.67-5.74 (m, 1H), 5.84-5.94 (m, 3H), 6.28 (s, 1H), 7.32 (s, 1H), 7.37 (dd, 1H), 7.79 (d, 1H), 7.84-7.94 (m, 2H), 8.47 (dd, 1H), 8.71 (s, 1H).

### Example 351

### 2′-[6-amino-5-(trifluoromethyl)pyridin-3-yl]-N-[cyclopropyl(pyridin-2-yl)methyl]-5′,6′-dihydrospiro[pyrrolidine-3,4′-pyrrolo[1,2-b]pyrazole]-1-carboxamide (Diastereomeric Mixture)

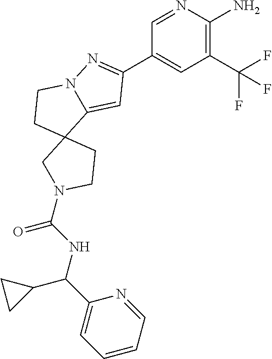 [SMILES: Nc1ncc(-c2cc3n(n2)CCC32CCN(C(=O)NC(c3ccccn3)C3CC3)C2)cc1C(F)(F)F]

Example 351 was prepared in analogy to Example 31 using 5-[5′,6′-dihydrospiro[pyrrolidine-3,4′-pyrrolo[1,2-b]pyrazol]-2′-yl]-3-(trifluoromethyl)pyridin-2-amine-hydrochloride salt (75.0 mg, 208 μmol) and (RS)-1-cyclopropyl-1-(pyridin-2-yl)methanamine (37.1 mg, 250 μmol).

LC-MS (method 1): Rt=1.00 min; MS (ESIpos): m/z=498 [M+H]⁺

1H-NMR (400 MHz, DMSO-d6): δ [ppm]=0.32-0.51 (m, 4H), 1.16-1.26 (m, 1H), 2.01-2.14 (m, 2H), 2.52-2.58 (m, 2H), 3.42-3.51 (m, 3H), 3.53-3.62 (m, 1H), 4.15-4.23 (m, 3H), 6.43-6.47 (m, 1H), 6.51-6.58 (m, 3H), 7.20-7.27 (m, 1H), 7.44 (dd, 1H), 7.73 (dtd, 1H), 8.00 (s, 1H), 8.46-8.52 (m, 1H), 8.58 (s, 1H)

### Example 352

### 2′-{6-amino-5-[(1S)-1-(pyridin-3-yl)ethoxy]pyridin-3-yl}-N-cyclobutyl-5′,6′-dihydrospiro[pyrrolidine-3,4′-pyrrolo[1,2-b]pyrazole]-1-carboxamide (Mixture of Stereoisomers)

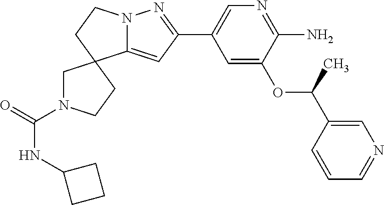 [SMILES: C[C@H](Oc1cc(-c2cc3n(n2)CCC32CCN(C(=O)NC3CCC3)C2)cnc1N)c1cccnc1]

Example 352 was prepared in analogy to Example 31 using 5-[5′,6′-dihydrospiro[pyrrolidine-3,4′-pyrrolo[1,2-b]pyrazol]-2′-yl]-3-[(1S)-1-(pyridin-3-yl)ethoxy]pyridin-2-amine-hydrochloride salt (75.0 mg, 182 μmol) and cyclobutanamine (15.5 mg, 218 μmol).

LC-MS (method 1): Rt=0.88 min; MS (ESIpos): m/z=474 [M+H]⁺

1H-NMR (400 MHz, DMSO-d6): δ [ppm]=1.47-1.62 (m, 5H), 1.85-2.15 (m, 6H), 3.36-3.43 (m, 3H), 3.44-3.52 (m, 1H), 4.05-4.21 (m, 3H), 5.71 (q, 1H), 5.89 (s, 2H), 6.27 (2s, 1H), 6.31 (d, 1H), 7.32 (s, 1H), 7.37 (dd, 1H), 7.87 (t, 1H), 7.91 (dt, 1H), 8.47 (dd, 1H), 8.69-8.72 (m, 1H).

### Example 353

### 2′-{6-amino-5-[(1S)-1-(pyridin-3-yl)ethoxy]pyridin-3-yl}-N-[2-(pyridin-4-yl)propan-2-yl]-5′,6′-dihydrospiro[pyrrolidine-3,4′-pyrrolo[1,2-b]pyrazole]-1-carboxamide (Mixture of Stereoisomers)

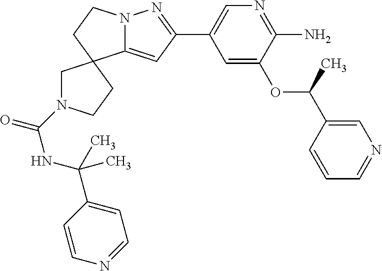 [SMILES: C[C@H](Oc1cc(-c2cc3n(n2)CCC32CCN(C(=O)NC(C)(C)c3ccncc3)C2)cnc1N)c1cccnc1]

Example 353 was prepared in analogy to Example 31 using 5-[5′,6′-dihydrospiro[pyrrolidine-3,4′-pyrrolo[1,2-b]pyrazol]-2′-yl]-3-[(1S)-1-(pyridin-3-yl)ethoxy]pyridin-2-amine-hydrochloride salt (75.0 mg, 182 μmol) and 2-(pyridin-4-yl)propan-2-amine (29.7 mg, 218 μmol).

LC-MS (method 1): Rt=0.54 min; MS (ESIpos): m/z=539 [M+H]⁺

1H-NMR (400 MHz, DMSO-d6): δ [ppm]=1.52 (s, 3H), 1.54 (s, 3H), 1.60 (d, 3H), 1.98-2.12 (m, 2H), 3.37-3.48 (m, 3H), 3.51-3.59 (m, 1H), 4.15 (t, 2H), 5.67-5.74 (m, 1H), 5.91 (s, 2H), 6.21 (d, 1H), 6.28 (s, 1H), 7.30-7.34 (m, 3H), 7.38 (dd, 1H), 7.88 (d, 1H), 7.91 (dt, 1H), 8.43 (ddd, 2H), 8.47 (dd, 1H), 8.71 (d, 1H).

### Example 354

### (rac)-2′-[6-amino-5-(trifluoromethyl)pyridin-3-yl]-N-[2-(2-cyanophenyl)propan-2-yl]-5′,6′-dihydrospiro[pyrrolidine-3,4′-pyrrolo[1,2-b]pyrazole]-1-carboxamide

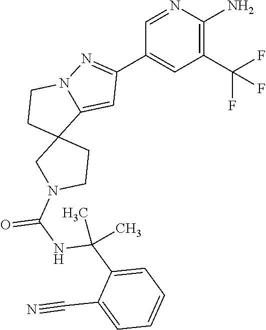 [SMILES: CC(C)(NC(=O)N1CCC2(CCn3nc(-c4cnc(N)c(C(F)(F)F)c4)cc32)C1)c1ccccc1C#N]

Example 354 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine-hydrochloride salt (75.0 mg, 208 μmol) and 2-(2-aminopropan-2-yl)benzonitrile (40.1 mg, 250 μmol).

LC-MS (method 1): Rt=1.34 min; MS (ESIpos): m/z=510 [M+H]⁺

1H-NMR (400 MHz, DMSO-d6): δ [ppm]=1.49 and 1.50 (2s, 6H), 2.04-2.17 (m, 2H), 2.54-2.65 (m, 2H), 3.49-3.69 (m, 2H), 3.75-3.92 (m, 2H), 4.15-4.27 (m, 2H), 6.50-6.55 (m, 3H), 7.41 and 7.47 (2ddd, 1H), 7.54-7.65 (m, 2H), 7.71 and 7.77 (2d, 1H), 8.02-8.05 (m, 1H), 8.61 (s, 1H), 9.94 and 9.97 (2s, 1H).

### Example 355

### [2′-{6-amino-5-[(1R)-1-(pyridin-3-yl)ethoxy]pyridin-3-yl}-5′,6′-dihydrospiro[pyrrolidine-3,4′-pyrrolo[1,2-b]pyrazol]-1-yl](1H-imidazol-1-yl)methanone (Mixture of Stereoisomers)

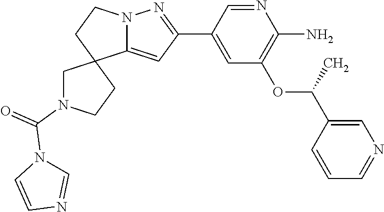 [SMILES: C[C@@H](Oc1cc(-c2cc3n(n2)CCC32CCN(C(=O)n3ccnc3)C2)cnc1N)c1cccnc1]

Example 355 was prepared in analogy to Example 31 using 5-[5′,6′-dihydrospiro[pyrrolidine-3,4′-pyrrolo[1,2-b]pyrazol]-2′-yl]-3-[(1R)-1-(pyridin-3-yl)ethoxy]pyridin-2-amine-hydrochloride salt (82.7 mg, 79% purity, 157 μmol) and 2-(1-methyl-1H-1,2,3-triazol-4-yl)propan-2-amine-hydrochloride salt (40.2 mg, 189 μmol).

LC-MS (method 1): Rt=0.79 min; MS (ESIpos): m/z=472 [M+H]⁺

1H-NMR (400 MHz, DMSO-d6): δ [ppm]=1.60 (d, 3H), 1.99-2.24 (m, 2H), 2.53-2.70 (m, 2H), 3.65-3.90 (m, 3H), 4.06-4.24 (m, 2H), 5.66-5.75 (m, 1H), 5.84-5.96 (m, 2H), 6.21-6.60 (m, 1H), 7.03 (br s, 1H), 7.32 (s, 1H), 7.37 (dd, 1H), 7.53-7.72 (m, 1H), 7.84-7.95 (m, 2H), 8.21 (br s, 1H), 8.44-8.50 (m, 1H), 8.71 (d, 1H)

### Example 356

### (rac)-2′-[6-amino-5-(trifluoromethyl)pyridin-3-yl]-N-(1-methylcyclobutyl)-5′,6′-dihydrospiro[pyrrolidine-3,4′-pyrrolo[1,2-b]pyrazole]-1-carboxamide

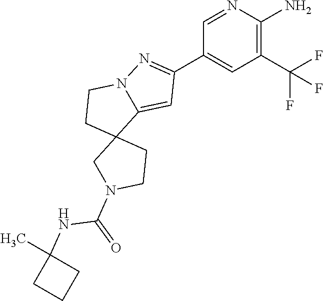 [SMILES: CC1(NC(=O)N2CCC3(CCn4nc(-c5cnc(N)c(C(F)(F)F)c5)cc43)C2)CCC1]

Example 356 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine-hydrochloride salt (120 mg, 334 μmol) and 1-methylcyclobutan-1-amine-hydrochloride salt (48.7 mg, 400 μmol).

LC-MS (method 1): Rt=1.04 min; MS (ESIpos): m/z=435 [M+H]⁺

¹H NMR (400 MHz, DMSO-d6) ppm: δ=8.59 (d, 1H), 8.01 (d, 1H), 6.54 (s, 2H), 6.47 (s, 1H), 5.97 (s, 1H), 5.76 (s, 1H), 4.18 (t, 2H), 3.46-3.55 (m, 1H), 3.35-3.43 (m, 3H), 2.52-2.55 (m, 2H), 2.24 (q, 2H), 1.99-2.10 (m, 2H), 1.85 (ddd, 2H), 1.73 (td, 2H), 1.38 (s, 3H)

### Example 357

### (rac)-2′-[6-amino-5-(trifluoromethyl)pyridin-3-yl]-N-(3-methyloxetan-3-yl)-5′,6′-dihydrospiro[pyrrolidine-3,4′-pyrrolo[1,2-b]pyrazole]-1-carboxamide

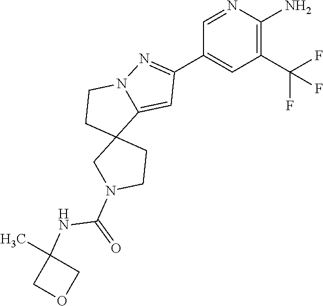 [SMILES: CC1(NC(=O)N2CCC3(CCn4nc(-c5cnc(N)c(C(F)(F)F)c5)cc43)C2)COC1]

Example 357 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine-hydrochloride salt (120 mg, 334 μmol) and 3-methyloxetan-3-amine (34.9 mg, 400 μmol).

LC-MS (method 1): Rt=0.85 min; MS (ESIpos): m/z=437 [M+H]⁺

¹H NMR (DMSO-d6) δ: 8.59 (d, 1H), 8.02 (d, 1H), 6.58 (s, 1H), 6.54 (s, 2H), 6.49 (s, 1H), 4.58 (dd, 2H), 4.24 (d, J=6.3 Hz, 2H), 4.19 (t, 2H), 3.47-3.56 (m, 1H), 3.37-3.45 (m, 3H), 2.52-2.57 (m, 2H), 2.00-2.13 (m, 2H), 1.52 (s, 3H)

### Example 358

### (rac)-2-[6-amino-5-(trifluoromethoxy)pyridin-3-yl]-N-[2-(3-fluoropyridin-2-yl)propan-2-yl]-6,7-dihydrospiro[pyrazolo[5,1-c][1,4]oxazine-4,3′-pyrrolidine]-1′-carboxamide

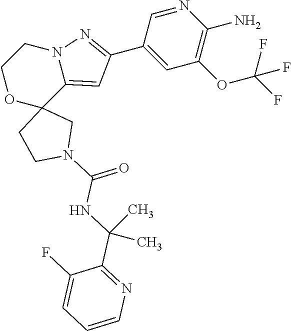 [SMILES: CC(C)(NC(=O)N1CCC2(C1)OCCn1nc(-c3cnc(N)c(OC(F)(F)F)c3)cc12)c1ncccc1F]

Example 358 was prepared in analogy to Example 31 using (rac)-5-[6,7-dihydrospiro[pyrazolo[5,1-c][1,4]oxazine-4,3′-pyrrolidin]-2-yl]-3-(trifluoromethoxy)pyridin-2-amine-hydrochloride salt (75.0 mg, 191 μmol) and 2-(3-fluoropyridin-2-yl)propan-2-amine (35.4 mg, 230 μmol).

LC-MS (method 1): Rt=1.04 min; MS (ESIpos): m/z=536 [M+H]⁺

1H-NMR (400 MHz, DMSO-d6) δ [ppm]: 1.013 (0.71), 1.030 (0.81), 1.049 (1.89), 1.067 (16.00), 1.084 (14.94), 1.623 (2.01), 1.631 (1.90), 2.522 (0.65), 2.706 (0.73), 2.724 (0.75), 3.219 (0.53), 3.235 (0.68), 3.251 (0.53), 4.118 (0.58), 4.130 (0.74), 6.468 (0.59), 6.556 (0.88), 6.715 (1.09), 7.744 (0.48), 8.250 (1.24), 8.318 (0.41), 8.322 (0.90), 8.327 (0.76).

### Example 359

### 2′-{6-amino-5-[(1S)-1-phenylethoxy]pyridin-3-yl}-N-[1-(pyridin-4-yl)cyclobutyl]-5′,6′-dihydrospiro[pyrrolidine-3,4′-pyrrolo[1,2-b]pyrazole]-1-carboxamide (Mixture of Stereoisomers)

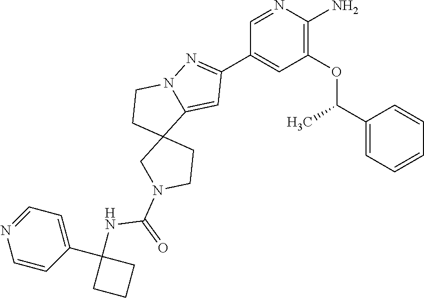 [SMILES: C[C@H](Oc1cc(-c2cc3n(n2)CCC32CCN(C(=O)NC3(c4ccncc4)CCC3)C2)cnc1N)c1ccccc1]

Example 359 was prepared in analogy to Example 31 using 5-[5′,6′-dihydrospiro[pyrrolidine-3,4′-pyrrolo[1,2-b]pyrazol]-2′-yl]-3-[(1S)-1-phenylethoxy]pyridin-2-amine-hydrochloride salt (40.0 mg, 97.1 μmol) and 1-(pyridin-4-yl)cyclobutan-1-amine (17.3 mg, 117 μmol).

LC-MS (method 1): Rₜ=1.05 min; MS (ESIpos): m/z=550 [M+H]⁺

¹H-NMR (400 MHz, DMSO-d6) δ [ppm]: 1.066 (0.77), 1.137 (4.54), 1.153 (4.61), 1.232 (0.41), 1.567 (11.65), 1.583 (11.48), 1.822 (0.97), 1.845 (1.13), 1.863 (0.76), 1.907 (0.59), 2.030 (2.72), 2.046 (2.49), 2.073 (2.10), 2.322 (1.06), 2.326 (1.44), 2.332 (1.20), 2.375 (2.13), 2.390 (2.19), 2.411 (2.22), 2.435 (2.25), 2.522 (3.85), 2.539 (6.22), 2.664 (0.91), 2.668 (1.25), 2.673 (0.87), 2.727 (13.23), 2.887 (16.00), 3.416 (7.40), 3.520 (1.46), 4.116 (2.88), 4.133 (5.40), 4.150 (2.83), 5.568 (1.52), 5.575 (1.55), 5.585 (1.61), 5.591 (1.53), 5.874 (4.88), 6.222 (6.87), 6.224 (6.21), 6.865 (3.68), 7.225 (1.44), 7.238 (6.15), 7.242 (8.15), 7.261 (2.46), 7.319 (3.85), 7.338 (7.06), 7.356 (3.74), 7.384 (3.82), 7.392 (4.76), 7.396 (4.94), 7.400 (4.75), 7.407 (4.07), 7.452 (5.52), 7.468 (3.67), 7.473 (4.58), 7.834 (3.67), 7.838 (6.09), 7.842 (3.75), 7.950 (2.15), 8.457 (3.95), 8.466 (5.07), 8.477 (3.03), 8.481 (3.62).

### Example 360

### 2′-{6-amino-5-[(1S)-1-phenylethoxy]pyridin-3-yl}-N-[2-(3-chloropyridin-4-yl)propan-2-yl]-5′,6′-dihydrospiro[pyrrolidine-3,4′-pyrrolo[1,2-b]pyrazole]-1-carboxamide (Mixture of Stereoisomers)

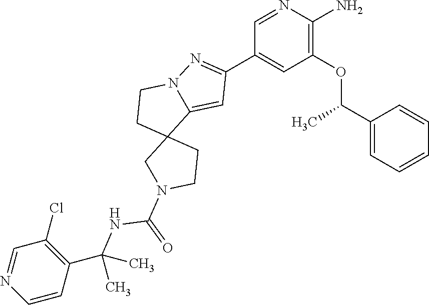 [SMILES: C[C@H](Oc1cc(-c2cc3n(n2)CCC32CCN(C(=O)NC(C)(C)c3ccncc3Cl)C2)cnc1N)c1ccccc1]

Example 360 was prepared in analogy to Example 31 using 5-[5′,6′-dihydrospiro[pyrrolidine-3,4′-pyrrolo[1,2-b]pyrazol]-2′-yl]-3-[(1S)-1-phenylethoxy]pyridin-2-amine-hydrochloride salt (40.0 mg, 97.1 μmol) and 2-(3-chloropyridin-4-yl)propan-2-amine (19.9 mg, 117 μmol).

LC-MS (method 1): Rₜ=1.10 min; MS (ESIpos): m/z=572 [M+H]⁺

¹H-NMR (400 MHz, DMSO-d6) δ [ppm]: 1.066 (0.54), 1.232 (0.55), 1.349 (0.44), 1.548 (0.83), 1.569 (4.52), 1.585 (4.45), 1.650 (14.14), 2.018 (0.83), 2.033 (0.87), 2.074 (1.33), 2.469 (1.90), 2.518 (3.79), 2.523 (2.65), 2.728 (3.66), 2.888 (4.60), 3.387 (6.75), 3.475 (1.97), 3.565 (1.04), 4.107 (1.35), 4.124 (2.62), 4.142 (1.32), 5.568 (0.75), 5.583 (0.75), 5.850 (3.14), 6.214 (4.57), 6.320 (1.70), 6.325 (1.56), 7.034 (16.00), 7.228 (2.76), 7.233 (2.50), 7.244 (1.85), 7.263 (1.33), 7.319 (1.94), 7.329 (0.66), 7.339 (3.46), 7.357 (1.70), 7.421 (1.63), 7.427 (1.42), 7.434 (1.39), 7.440 (1.47), 7.454 (2.87), 7.475 (2.09), 7.536 (0.51), 7.549 (0.67), 7.565 (0.42), 7.596 (0.44), 7.625 (0.45), 7.684 (6.45), 7.831 (1.82), 7.835 (2.72), 7.839 (1.73), 7.950 (0.57), 8.138 (0.75), 8.385 (1.63), 8.392 (1.77), 8.399 (1.66), 8.405 (1.58), 8.416 (3.43), 8.420 (3.27), 8.568 (0.40), 8.581 (0.40), 8.611 (0.88).

### Example 361

### 2′-{6-amino-5-[(1R)-1-phenylethoxy]pyridin-3-yl}-N-[1-(pyridin-4-yl)cyclobutyl]-5′,6′-dihydrospiro[pyrrolidine-3,4′-pyrrolo[1,2-b]pyrazole]-1-carboxamide (Mixture of Stereoisomers)

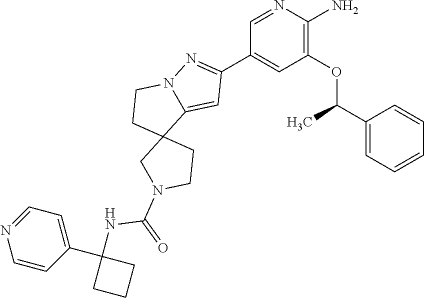 [SMILES: C[C@@H](Oc1cc(-c2cc3n(n2)CCC32CCN(C(=O)NC3(c4ccncc4)CCC3)C2)cnc1N)c1ccccc1]

Example 361 was prepared in analogy to Example 31 using 5-[5′,6′-dihydrospiro[pyrrolidine-3,4′-pyrrolo[1,2-b]pyrazol]-2′-yl]-3-[(1R)-1-phenylethoxy]pyridin-2-amine-hydrochloride salt (75.0 mg, 182 μmol) and 1-(pyridin-4-yl)cyclobutan-1-amine (32.4 mg, 218 μmol).

LC-MS (method 1): Rₜ=1.05 min; MS (ESIpos): m/z=550 [M+H]⁺

¹H-NMR (400 MHz, DMSO-d6) δ [ppm]: 1.067 (0.41), 1.552 (0.46), 1.568 (5.72), 1.584 (5.40), 1.753 (1.22), 1.824 (0.45), 1.846 (0.51), 1.995 (0.55), 2.003 (0.67), 2.018 (0.93), 2.031 (1.24), 2.047 (1.16), 2.376 (0.92), 2.391 (0.97), 2.414 (0.95), 2.437 (1.00), 2.463 (0.94), 2.523 (0.92), 2.727 (12.68), 2.729 (12.64), 2.887 (16.00), 3.388 (2.02), 3.418 (3.29), 3.504 (0.51), 3.521 (0.68), 4.117 (1.34), 4.135 (2.50), 4.152 (1.34), 5.572 (0.70), 5.578 (0.72), 5.587 (0.73), 5.594 (0.70), 5.888 (1.62), 6.224 (3.30), 6.227 (2.95), 6.869 (1.64), 7.226 (0.57), 7.243 (3.54), 7.263 (1.16), 7.279 (0.59), 7.319 (1.80), 7.339 (3.12), 7.357 (1.64), 7.386 (1.89), 7.390 (1.55), 7.393 (2.28), 7.398 (2.39), 7.402 (2.32), 7.405 (1.68), 7.409 (2.10), 7.454 (2.53), 7.470 (1.69), 7.474 (2.17), 7.837 (1.73), 7.841 (2.91), 7.845 (1.87), 7.951 (2.09), 8.459 (1.90), 8.463 (1.35), 8.468 (2.31), 8.471 (2.15), 8.474 (2.27), 8.478 (1.39), 8.483 (1.86).

### Example 362

### 2′-{6-amino-5-[(1R)-1-phenylethoxy]pyridin-3-yl}-N-[2-(3-chloropyridin-4-yl)propan-2-yl]-5′,6′-dihydrospiro[pyrrolidine-3,4′-pyrrolo[1,2-b]pyrazole]-1-carboxamide (Mixture of Stereoisomers)

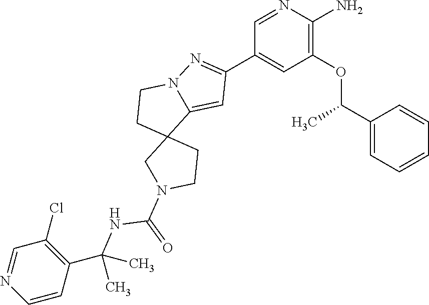 [SMILES: C[C@H](Oc1cc(-c2cc3n(n2)CCC32CCN(C(=O)NC(C)(C)c3ccncc3Cl)C2)cnc1N)c1ccccc1]

Example 362 was prepared in analogy to Example 31 using 5-[5′,6′-dihydrospiro[pyrrolidine-3,4′-pyrrolo[1,2-b]pyrazol]-2′-yl]-3-[(1R)-1-phenylethoxy]pyridin-2-amine-hydrochloride salt (75.0 mg, 182 μmol) and 2-(3-chloropyridin-4-yl)propan-2-amine (37.3 mg, 218 μmol).

LC-MS (method 1): Rₜ=1.10 min; MS (ESIpos): m/z=572 [M+H]⁺

¹H-NMR (400 MHz, DMSO-d6) δ [ppm]: 1.066 (1.67), 1.156 (1.90), 1.561 (0.64), 1.574 (3.10), 1.589 (3.04), 1.650 (7.29), 2.020 (0.59), 2.034 (0.60), 2.454 (0.57), 2.471 (1.16), 2.518 (1.16), 2.523 (0.77), 2.729 (13.52), 2.888 (16.00), 3.389 (2.10), 4.111 (0.87), 4.128 (1.76), 4.145 (0.87), 5.585 (0.46), 5.590 (0.47), 5.601 (0.48), 5.607 (0.46), 5.981 (0.65), 6.226 (2.71), 6.319 (1.07), 6.325 (1.07), 7.229 (0.45), 7.247 (1.33), 7.254 (1.73), 7.258 (1.63), 7.266 (0.98), 7.322 (1.41), 7.341 (2.41), 7.355 (0.54), 7.359 (1.16), 7.421 (1.00), 7.428 (1.11), 7.434 (0.91), 7.440 (0.93), 7.457 (1.90), 7.478 (1.43), 7.827 (1.24), 7.831 (1.88), 7.835 (1.18), 7.951 (2.04), 8.386 (1.13), 8.392 (1.27), 8.399 (1.16), 8.406 (1.12), 8.416 (2.30), 8.420 (2.32).

### Example 363

### (rac)-2-[6-amino-5-(trifluoromethoxy)pyridin-3-yl]-N-[2-(pyridin-2-yl)propan-2-yl]-6,7-dihydrospiro[pyrazolo[5,1-c][1,4]oxazine-4,3′-pyrrolidine]-1′-carboxamide

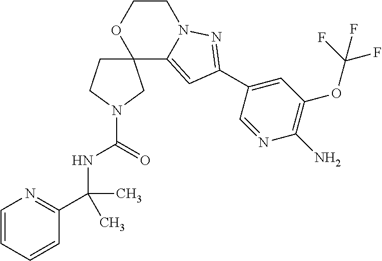 [SMILES: CC(C)(NC(=O)N1CCC2(C1)OCCn1nc(-c3cnc(N)c(OC(F)(F)F)c3)cc12)c1ccccn1]

Example 363 was prepared in analogy to Example 31 using (rac)-5-[6,7-dihydrospiro[pyrazolo[5,1-c][1,4]oxazine-4,3′-pyrrolidin]-2-yl]-3-(trifluoromethoxy)pyridin-2-amine (75.0 mg, 211 μmol) and 2-(pyridin-2-yl)propan-2-amine (34.5 mg, 253 μmol).

¹H-NMR (400 MHz, DMSO-d6) δ [ppm]: 1.137 (1.06), 1.233 (1.00), 1.256 (0.46), 1.578 (6.63), 1.584 (6.51), 2.116 (0.48), 2.323 (0.48), 2.327 (0.72), 2.332 (0.62), 2.345 (0.40), 2.518 (1.77), 2.523 (1.25), 2.540 (16.00), 2.669 (0.48), 3.408 (0.43), 3.499 (0.50), 3.528 (0.59), 3.620 (0.56), 3.796 (0.67), 3.824 (0.57), 4.109 (0.68), 4.130 (2.19), 4.140 (1.66), 6.425 (1.68), 6.557 (2.69), 6.729 (3.53), 7.171 (0.64), 7.174 (0.67), 7.183 (0.67), 7.186 (0.76), 7.190 (0.76), 7.192 (0.69), 7.202 (0.72), 7.204 (0.72), 7.440 (1.21), 7.459 (1.31), 7.698 (0.70), 7.702 (0.73), 7.716 (0.86), 7.721 (0.86), 7.736 (0.61), 7.740 (0.79), 7.746 (1.12), 7.750 (1.46), 7.754 (0.98), 8.329 (2.33), 8.334 (2.32), 8.449 (0.79), 8.451 (0.90), 8.453 (0.92), 8.455 (0.81), 8.461 (0.80), 8.463 (0.90), 8.465 (0.84), 8.467 (0.72).

### Example 364

### 2′-{6-amino-5-[(1R)-1-(pyridin-2-yl)ethoxy]pyridin-3-yl}-N-[2-(3-chloropyridin-4-yl)propan-2-yl]-5′,6′-dihydrospiro[pyrrolidine-3,4′-pyrrolo[1,2-b]pyrazole]-1-carboxamide (Mixture of Stereoisomers)

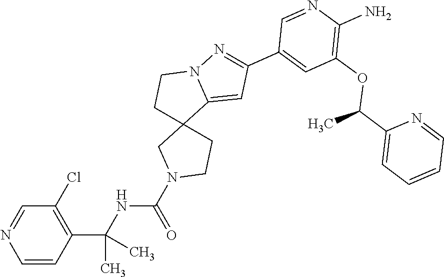 [SMILES: C[C@@H](Oc1cc(-c2cc3n(n2)CCC32CCN(C(=O)NC(C)(C)c3ccncc3Cl)C2)cnc1N)c1ccccn1]

Example 364 was prepared in analogy to Example 31 using 5-[5′,6′-dihydrospiro[pyrrolidine-3,4′-pyrrolo[1,2-b]pyrazol]-2′-yl]-3-[(1R)-1-(pyridin-2-yl)ethoxy]pyridin-2-amine-hydrochloride salt (120 mg, 291 μmol) and 2-(3-chloropyridin-4-yl)propan-2-amine (59.5 mg, 349 μmol).

LC-MS (method 1): Rₜ=0.96 min; MS (ESIpos): m/z=573 [M+H]⁺

¹H-NMR (400 MHz, DMSO-d6) δ [ppm]: 1.065 (4.29), 1.155 (2.81), 1.206 (0.93), 1.222 (1.17), 1.231 (0.69), 1.239 (1.24), 1.247 (3.20), 1.257 (2.27), 1.263 (3.31), 1.271 (3.05), 1.276 (1.34), 1.288 (2.87), 1.347 (0.46), 1.409 (1.34), 1.535 (2.51), 1.551 (3.02), 1.559 (8.01), 1.629 (5.56), 1.648 (16.00), 1.769 (7.60), 2.021 (0.97), 2.030 (1.04), 2.038 (0.97), 2.518 (3.67), 2.523 (2.51), 3.112 (0.42), 3.123 (0.42), 3.131 (0.43), 3.141 (0.41), 3.383 (4.69), 3.473 (0.77), 3.565 (0.63), 4.114 (1.47), 4.132 (2.90), 4.149 (1.50), 5.562 (1.09), 5.578 (1.09), 5.633 (0.45), 5.649 (0.45), 6.128 (0.68), 6.245 (3.74), 6.247 (4.04), 6.324 (3.10), 6.542 (1.18), 7.103 (0.96), 7.107 (1.00), 7.281 (2.48), 7.284 (2.43), 7.294 (1.27), 7.296 (1.28), 7.300 (1.23), 7.303 (1.14), 7.313 (1.10), 7.315 (1.07), 7.336 (1.06), 7.350 (1.03), 7.420 (1.86), 7.423 (1.93), 7.433 (1.95), 7.436 (1.98), 7.455 (1.56), 7.459 (1.00), 7.467 (0.96), 7.471 (1.49), 7.507 (1.72), 7.527 (3.31), 7.532 (1.80), 7.776 (1.11), 7.781 (1.15), 7.795 (1.77), 7.800 (1.82), 7.814 (0.90), 7.819 (0.90), 7.849 (2.17), 7.852 (3.04), 7.856 (2.28), 8.316 (1.49), 8.329 (1.39), 8.384 (2.36), 8.390 (2.28), 8.398 (2.17), 8.403 (2.02), 8.419 (4.33), 8.425 (2.73), 8.434 (3.93), 8.545 (2.85), 8.549 (2.42), 8.557 (2.37), 8.560 (2.71), 8.636 (1.00), 8.649 (0.95), 8.685 (1.82).

### Example 365

### 2′-{6-amino-5-[(1R)-1-(pyridin-3-yl)ethoxy]pyridin-3-yl}-N-[2-(3-chloropyridin-4-yl)propan-2-yl]-5′,6′-dihydrospiro[pyrrolidine-3,4′-pyrrolo[1,2-b]pyrazole]-1-carboxamide (Mixture of Stereoisomers)

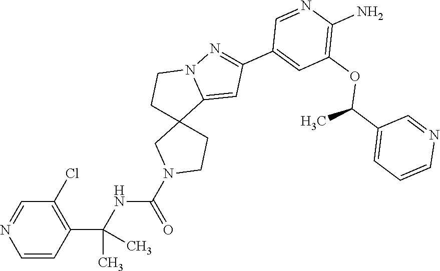 [SMILES: C[C@@H](Oc1cc(-c2cc3n(n2)CCC32CCN(C(=O)NC(C)(C)c3ccncc3Cl)C2)cnc1N)c1cccnc1]

Example 365 was prepared in analogy to Example 31 using 5-[5′,6′-dihydrospiro[pyrrolidine-3,4′-pyrrolo[1,2-b]pyrazol]-2′-yl]-3-[(1R)-1-(pyridin-3-yl)ethoxy]pyridin-2-amine-hydrochloride salt (210 mg, 509 μmol) and 2-(3-chloropyridin-4-yl)propan-2-amine (104 mg, 610 μmol).

LC-MS (method 1): Rₜ=0.91 min; MS (ESIpos): m/z=573 [M+H]⁺

¹H-NMR (400 MHz, DMSO-d6) δ [ppm]: 1.052 (0.59), 1.222 (0.42), 1.230 (0.91), 1.498 (5.44), 1.595 (6.96), 1.611 (7.01), 1.650 (16.00), 1.708 (4.96), 2.026 (1.15), 2.040 (1.21), 2.048 (0.99), 2.058 (0.61), 2.067 (0.50), 2.083 (0.64), 2.459 (1.09), 2.468 (1.13), 2.476 (2.31), 2.518 (1.70), 2.523 (1.10), 3.166 (13.81), 3.447 (1.48), 3.464 (1.14), 3.483 (1.19), 4.119 (1.83), 4.137 (3.72), 4.154 (1.77), 5.696 (1.01), 5.700 (1.03), 5.711 (1.04), 5.716 (1.00), 5.915 (4.77), 6.256 (4.78), 6.258 (5.16), 6.326 (2.25), 6.332 (2.28), 7.315 (3.27), 7.320 (3.26), 7.359 (1.50), 7.361 (1.52), 7.371 (1.54), 7.373 (1.55), 7.379 (1.61), 7.381 (1.60), 7.391 (1.62), 7.393 (1.59), 7.422 (1.84), 7.426 (1.88), 7.434 (1.92), 7.440 (1.85), 7.860 (2.92), 7.863 (3.43), 7.864 (3.36), 7.866 (2.87), 7.901 (1.05), 7.906 (1.68), 7.911 (1.07), 7.920 (0.99), 7.925 (1.59), 7.929 (0.99), 8.386 (2.96), 8.391 (3.00), 8.399 (2.68), 8.404 (2.61), 8.417 (5.15), 8.425 (5.10), 8.451 (0.56), 8.463 (2.89), 8.467 (2.77), 8.475 (2.69), 8.479 (2.49), 8.486 (0.75), 8.707 (2.94), 8.712 (2.92).

### Example 366

### 2′-{6-amino-5-[(1R)-1-(pyridin-4-yl)ethoxy]pyridin-3-yl}-N-[2-(3-chloropyridin-4-yl)propan-2-yl]-5′,6′-dihydrospiro[pyrrolidine-3,4′-pyrrolo[1,2-b]pyrazole]-1-carboxamide (Mixture of Stereoisomers)

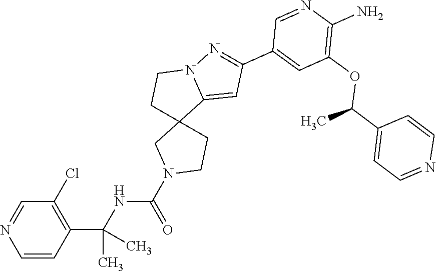 [SMILES: C[C@@H](Oc1cc(-c2cc3n(n2)CCC32CCN(C(=O)NC(C)(C)c3ccncc3Cl)C2)cnc1N)c1ccncc1]

Example 366 was prepared in analogy to Example 31 using 5-[5′,6′-dihydrospiro[pyrrolidine-3,4′-pyrrolo[1,2-b]pyrazol]-2′-yl]-3-[(1R)-1-(pyridin-4-yl)ethoxy]pyridin-2-amine-hydrochloride salt (250 mg, 605 μmol) and 2-(3-chloropyridin-4-yl)propan-2-amine (124 mg, 727 μmol).

LC-MS (method 1): Rₜ=0.90 min; MS (ESIpos): m/z=573 [M+H]⁺

¹H-NMR (400 MHz, DMSO-d6) δ [ppm]: 1.497 (0.44), 1.561 (3.39), 1.575 (8.11), 1.647 (8.44), 2.017 (0.62), 2.033 (0.63), 2.451 (0.57), 2.459 (0.54), 2.467 (1.11), 2.476 (1.07), 2.518 (0.63), 2.523 (0.46), 3.165 (16.00), 3.336 (0.73), 3.428 (1.04), 3.446 (0.61), 3.459 (0.51), 3.463 (0.51), 3.476 (0.58), 3.564 (2.02), 4.105 (0.97), 4.123 (1.96), 4.140 (0.93), 5.642 (0.59), 5.657 (0.59), 5.949 (2.47), 6.234 (3.94), 6.333 (1.25), 6.337 (1.22), 7.232 (1.53), 7.420 (0.97), 7.425 (0.97), 7.433 (1.02), 7.438 (0.93), 7.475 (2.71), 7.479 (1.62), 7.486 (1.66), 7.490 (2.74), 7.867 (1.41), 7.870 (2.03), 7.875 (1.43), 8.385 (1.51), 8.389 (1.44), 8.398 (1.46), 8.402 (1.36), 8.415 (3.48), 8.532 (3.71), 8.536 (2.00), 8.543 (2.04), 8.547 (3.39).

### Example 367

### 2′-(6-amino-5-cyanopyridin-3-yl)-N-[(1R)-1-(pyridin-4-yl)ethyl]-5′,6′-dihydrospiro[pyrrolidine-3,4′-pyrrolo[1,2-b]pyrazole]-1-carboxamide (Isomer 1)

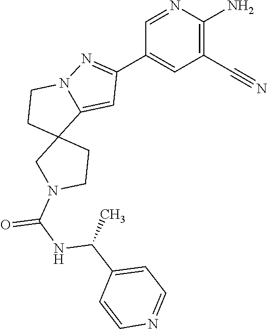 [SMILES: C[C@@H](NC(=O)N1CCC2(CCn3nc(-c4cnc(N)c(C#N)c4)cc32)C1)c1ccncc1]

Example 367 was prepared in analogy to Example 31 using (rac)-2-amino-5-[5′,6′-dihydrospiro[pyrrolidine-3,4′-pyrrolo[1,2-b]pyrazol]-2′-yl]pyridine-3-carbonitrile-hydrochloride salt (150 mg, 90% purity, 426 μmol) and (1R)-1-(pyridin-4-yl)ethan-1-amine (62.5 mg, 511 μmol).

The mixture of isomers was then separated by chiral HPLC to give 30.0 mg (98% purity, 16% yield) of the title compound.

Instrument: PrepCon Labomatic HPLC-3; Column: YMC Cellulose SB 10μ, 250×50; eluent A: methyl tert-butyl ether+0.1 vol % diethylamine; eluent B: acetonitrile+0.1 vol % diethylamine; isocratic: 50% A+50% B; flow: 100 mL/min; temperature: 25° C.; UV: 280 nm

Retention time: 7.4-8.9 min

LC-MS (method 1): Rt=0.76 min; MS (ESIpos): m/z=429 [M+H]⁺

¹H-NMR (400 MHz, DMSO-d₆): δ [ppm]=1.37 (d, 3H), 2.07 (br t, 2H), 2.52-2.58 (m, 2H), 3.41-3.60 (m, 4H), 4.19 (t, 2H), 4.81 (quin, 1H), 6.45 (s, 1H), 6.61 (d, 1H), 6.99 (s, 2H), 7.29-7.35 (m, 2H), 8.15 (d, 1H), 8.43-8.49 (m, 2H), 8.62 (d, 1H).

α²⁰_{D}: −96.4° (c=1, in MeOH)

### Example 368

### 2′-(6-amino-5-cyanopyridin-3-yl)-N-[(1R)-1-(pyridin-4-yl)ethyl]-5′,6′-dihydrospiro[pyrrolidine-3,4′-pyrrolo[1,2-b]pyrazole]-1-carboxamide (Isomer 2)

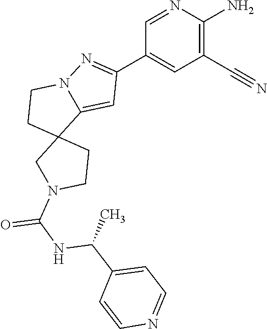 [SMILES: C[C@@H](NC(=O)N1CCC2(CCn3nc(-c4cnc(N)c(C#N)c4)cc32)C1)c1ccncc1]

The mixture of isomers was separated by chiral HPLC to give 35.0 mg (100% purity, 18% yield) of the title compound.

Instrument: PrepCon Labomatic HPLC-3; Column: YMC Cellulose SB 10μ, 250×50; eluent A: methyl tert-butyl ether+0.1 vol % diethylamine; eluent B: acetonitrile+0.1 vol % diethylamine; isocratic: 50% A+50% B; flow: 100 mL/min; temperature: 25° C.; UV: 280 nm

Retention time: 12.2-14.7 min

α²⁰_{D}: +61.3° (c=1, in MeOH)

### Example 369

### 2′-(6-amino-5-cyanopyridin-3-yl)-N-[1-(pyridin-4-yl)cyclobutyl]-5′,6′-dihydrospiro[pyrrolidine-3,4′-pyrrolo[1,2-b]pyrazole]-1-carboxamide (Enantiomer 1)

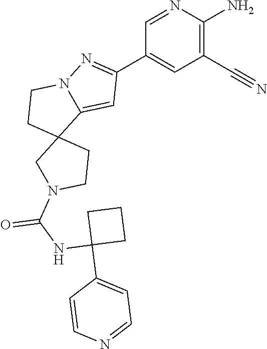 [SMILES: N#Cc1cc(-c2cc3n(n2)CCC32CCN(C(=O)NC3(c4ccncc4)CCC3)C2)cnc1N]

Example 369 was prepared in analogy to Example 31 using (rac)-2-amino-5-[5′,6′-dihydrospiro[pyrrolidine-3,4′-pyrrolo[1,2-b]pyrazol]-2′-yl]pyridine-3-carbonitrile-hydrochloride salt (150 mg, 90% purity, 426 μmol) and 1-(pyridin-4-yl)cyclobutan-1-amine (75.8 mg, 511 μmol). The racemate (rac)-2-amino-5-[5′,6′-dihydrospiro[pyrrolidine-3,4′-pyrrolo[1,2-b]pyrazol]-2′-yl]pyridine-3-carbonitrile-hydrochloride salt (150 mg, 90% purity, 426 μmol) was then separated by chiral HPLC to give 21.3 mg (95% purity, 10% yield) of the title compound.

Instrument: PrepCon Labomatic HPLC-3; Column: YMC Cellulose SB 10μ, 250×50; eluent A: methyl tert-butyl ether+0.1 vol % diethylamine; eluent B: acetonitrile+0.1 vol % diethylamine; isocratic: 10% A+90% B; flow: 100 mL/min; temperature: 25° C.; UV: 254 nm

Retention time: 9.0-10.7 min;

LC-MS (method 1): Rt=0.83 min; MS (ESIpos): m/z=455 [M+H]⁺

1H-NMR (400 MHz, CHLOROFORM-d): δ [ppm]=1.94 (br d, 1H), 2.07-2.20 (m, 2H), 2.22-2.32 (m, 1H), 2.44-2.53 (m, 2H), 2.54-2.74 (m, 4H), 3.48-3.59 (m, 3H), 3.65 (dq, 1H), 4.27 (t, 2H), 4.83 (s, 1H), 5.21 (s, 2H), 6.15 (s, 1H), 7.33-7.43 (m, 2H), 8.07 (d, 1H), 8.58 (br d, 2H), 8.62 (d, 1H).

α²⁰_{D}: −63.2° (c=1, in CHCl3)

### Example 370 (3R)-2′-[6-amino-5-(trifluoromethyl)pyridin-3-yl]-N-[(1S)-1-(pyridin-4-yl)ethyl]-5′,6′-dihydrospiro[pyrrolidine-3,4′-pyrrolo[1,2-b]pyrazole]-1-carboxamide

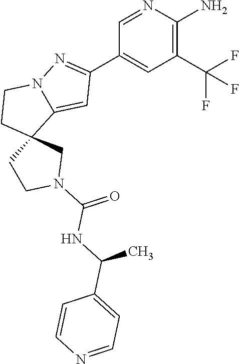 [SMILES: C[C@H](NC(=O)N1CC[C@@]2(CCn3nc(-c4cnc(N)c(C(F)(F)F)c4)cc32)C1)c1ccncc1]

Example 370 was prepared in analogy to Example 31 using 5-[(3R)-5′,6′-dihydrospiro[pyrrolidine-3,4′-pyrrolo[1,2-b]pyrazol]-2′-yl]-3-(trifluoromethyl)pyridin-2-amine-hydrochloride salt (60.0 mg, 84% purity, 140 μmol) and (1S)-1-(pyridin-4-yl)ethan-1-amine (20.5 mg, 168 μmol).

α²⁰_{D}: +60.6° (c=1, in CHCl3)

1H-NMR (400 MHz, CHLOROFORM-d): δ [ppm]=1.47-1.53 (m, 3H), 2.12 (ddd, 1H), 2.23-2.33 (m, 1H), 2.54-2.73 (m, 2H), 3.52-3.63 (m, 3H), 3.68 (br dd, 1H), 4.28 (t, 2H), 4.45 (br d, 1H), 4.94-5.08 (m, 3H), 6.19 (s, 1H), 7.17-7.38 (m, 6H), 8.10 (d, 1H), 8.48-8.63 (m, 3H).

### Example 371

### (3S)-2′-[6-amino-5-(trifluoromethyl)pyridin-3-yl]-N-[(1S)-1-(pyridin-4-yl)ethyl]-5′,6′-dihydrospiro[pyrrolidine-3,4′-pyrrolo[1,2-b]pyrazole]-1-carboxamide

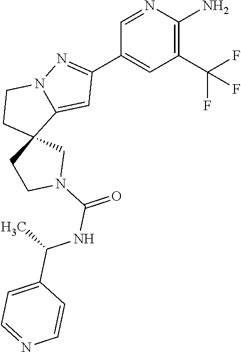 [SMILES: C[C@H](NC(=O)N1CC[C@]2(CCn3nc(-c4cnc(N)c(C(F)(F)F)c4)cc32)C1)c1ccncc1]

Example 371 was prepared in analogy to Example 31 using 5-[(3S)-5′,6′-dihydrospiro[pyrrolidine-3,4′-pyrrolo[1,2-b]pyrazol]-2′-yl]-3-(trifluoromethyl)pyridin-2-amine-hydrochloride salt (60.0 mg, 84% purity, 140 μmol) and (1S)-1-(pyridin-4-yl)ethan-1-amine (20.5 mg, 168 μmol).

α²⁰_{D}: −40.9° (c=1, in CHCl3)

1H-NMR (400 MHz, CHLOROFORM-d): δ [ppm]=1.51 (d, 3H), 2.06-2.19 (m, 1H), 2.22-2.35 (m, 1H), 2.54-2.75 (m, 2H), 3.52-3.64 (m, 3H), 3.64-3.73 (m, 1H), 4.28 (t, 2H), 4.46 (br d, 1H), 4.92-5.14 (m, 3H), 6.20 (s, 1H), 7.24 (d, 1H), 8.10 (d, 1H), 8.48-8.63 (m, 3H).

### Example 372

### 2′-(6-amino-5-cyanopyridin-3-yl)-N-[(1S)-1-(pyridin-4-yl)ethyl]-5′,6′-dihydrospiro[pyrrolidine-3,4′-pyrrolo[1,2-b]pyrazole]-1-carboxamide (Isomer 1)

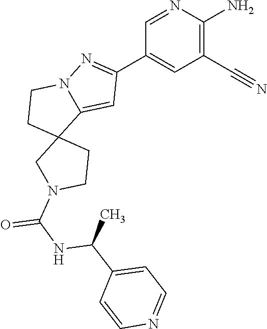 [SMILES: C[C@H](NC(=O)N1CCC2(CCn3nc(-c4cnc(N)c(C#N)c4)cc32)C1)c1ccncc1]

Example 372 was prepared in analogy to Example 31 using (rac)-2-amino-5-[5′,6′-dihydrospiro[pyrrolidine-3,4′-pyrrolo[1,2-b]pyrazol]-2′-yl]pyridine-3-carbonitrile-hydrochloride salt (113 mg, 90% purity, 320 μmol) and (1S)-1-(pyridin-4-yl)ethan-1-amine (43.0 mg, 352 μmol). The mixture of isomers was separated by chiral HPLC to give 2.6 mg (95% purity, 2% yield) of the title compound.

Instrument: PrepCon Labomatic HPLC-3; Column: YMC Cellulose SB 10μ, 250×50; eluent A: methyl tert-butyl ether+0.1 vol % diethylamine; eluent B: acetonitrile; isocratic: 70% A+30% B; flow: 100 mL/min; temperature: 25° C.; UV: 254 nm;

Retention time: 14.26-15.98 min;

α²⁰_{D}: −50.5° (c=1, in MeOH)

¹H-NMR (400 MHz, DMSO-d6) δ [ppm]: 0.848 (0.50), 1.170 (0.40), 1.229 (2.25), 1.360 (5.24), 1.378 (5.26), 2.053 (0.96), 2.071 (1.82), 2.088 (0.93), 2.518 (1.77), 2.523 (1.70), 2.526 (1.61), 2.543 (2.18), 2.561 (1.47), 3.322 (0.52), 3.329 (0.74), 3.336 (0.81), 3.340 (0.88), 3.412 (1.20), 3.419 (0.87), 3.424 (1.16), 3.429 (0.85), 3.454 (2.54), 3.468 (2.53), 3.486 (1.07), 3.493 (0.87), 3.502 (0.63), 3.515 (0.54), 3.532 (0.99), 3.548 (0.59), 3.558 (0.56), 4.166 (1.49), 4.184 (2.46), 4.201 (1.47), 4.791 (0.68), 4.810 (1.04), 4.828 (0.67), 5.748 (16.00), 6.449 (7.65), 6.610 (1.28), 6.630 (1.22), 6.984 (4.01), 7.327 (1.48), 8.144 (3.69), 8.149 (3.95), 8.469 (0.45), 8.611 (3.27), 8.617 (3.20).

### Example 373

### 2′-(6-amino-5-cyanopyridin-3-yl)-N-[(1S)-1-(pyridin-4-yl)ethyl]-5′,6′-dihydrospiro[pyrrolidine-3,4′-pyrrolo[1,2-b]pyrazole]-1-carboxamide (Isomer 2)

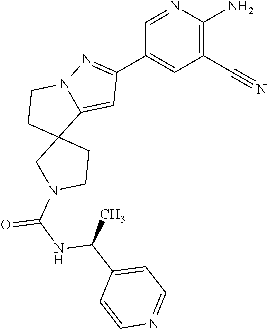 [SMILES: C[C@H](NC(=O)N1CCC2(CCn3nc(-c4cnc(N)c(C#N)c4)cc32)C1)c1ccncc1]

The mixture of isomers was separated by chiral HPLC to give 3.50 mg (95% purity, 2% yield) of the title compound.

Instrument: PrepCon Labomatic HPLC-3; Column: YMC Cellulose SB 10μ, 250×50; eluent A: methyl tert-butyl ether+0.1 vol % diethylamine; eluent B: acetonitrile; isocratic: 70% A+30% B; flow: 100 mL/min; temperature: 25° C.; UV: 254 nm;

Retention time: 20.07-24.33 min;

α²⁰_{D}: 67.9° (c=1, in MeOH)

### Example 374

### 2′-[6-amino-5-(trifluoromethyl)pyridin-3-yl]-N-[(1S)-1-(pyridin-4-yl)ethyl]-5′,6′-dihydrospiro[azetidine-3,4′-pyrrolo[1,2-b]pyrazole]-1-carboxamide

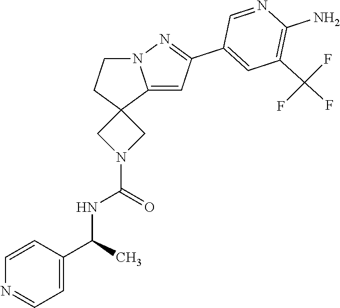 [SMILES: C[C@H](NC(=O)N1CC2(CCn3nc(-c4cnc(N)c(C(F)(F)F)c4)cc32)C1)c1ccncc1]

Example 374 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine-hydrochloride salt (100 mg, 57% purity, 165 μmol) and (1S)-1-(pyridin-4-yl)ethan-1-amine (30.2 mg, 247 μmol).

1H NMR (400 MHz, DMSO-d6) δ ppm 8.62 (d, J=1.5 Hz, 1H) 8.46-8.53 (m, 1H) 8.04 (d, J=2.3 Hz, 1H) 7.30-7.37 (m, 2H) 7.01 (d, J=8.1 Hz, 1H, NH) 6.74 (s, 1H) 6.55 (s, 2H, NH2) 4.79 (quin, J=7.4 Hz, 1H) 4.13 (t, J=7.0 Hz, 2H) 3.96-4.10 (m, 4H) 2.86 (t, J=6.8 Hz, 2H) 1.36 (d, J=7.1 Hz, 3H)

LC-MS (method 1): Rₜ=0.86 min; MS (ESIpos): m/z=459 [M+H]⁺

### Example 375

### 2′-[6-amino-5-(trifluoromethyl)pyridin-3-yl]-N-[(1R)-1-(pyridin-4-yl)ethyl]-5′,6′-dihydrospiro[azetidine-3,4′-pyrrolo[1,2-b]pyrazole]-1-carboxamide

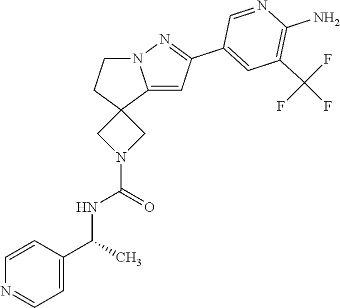 [SMILES: C[C@@H](NC(=O)N1CC2(CCn3nc(-c4cnc(N)c(C(F)(F)F)c4)cc32)C1)c1ccncc1]

Example 375 was prepared in analogy to Example 31 using 5-(5′,6′-dihydrospiro[azetidine-3,4′-pyrrolo[1,2-b]pyrazol]-2′-yl)-3-(trifluoromethyl)pyridin-2-amine-hydrochloride salt (100 mg, 57% purity, 165 μmol) and (1R)-1-(pyridin-4-yl)ethan-1-amine-hydrochloride salt (39.2 mg, 247 μmol).

1H NMR (400 MHz, DMSO-d6) δ ppm 8.62 (d, J=1.5 Hz, 1H) 8.48-8.53 (m, 2H) 8.04 (d, J=2.0 Hz, 1H) 7.31-7.36 (m, 2H) 7.21-7.27 (m, 1H) 7.01 (d, J=8.1 Hz, 1H, NH) 6.74 (s, 1H) 6.55 (s, 2H, NH2) 4.79 (m, 1H) 4.13 (t, J=6.8 Hz, 2H) 3.99-4.11 (m, 4H) 2.86 (t, J=6.8 Hz, 2H) 1.36 (d, J=7.1 Hz, 3H)

LC-MS (method 1): Rₜ=0.86 min; MS (ESIpos): m/z=458 [M+H]⁺

### Example 376

### (rac)-2′-[6-amino-5-(trifluoromethyl)pyridin-3-yl]-N-[1-(2-chlorophenyl)cyclobutyl]-5′,6′-dihydrospiro[pyrrolidine-3,4′-pyrrolo[1,2-b]pyrazole]-1-carboxamide

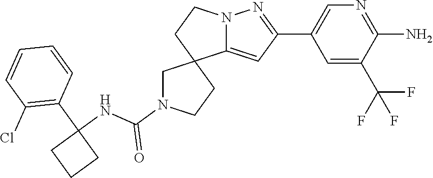 [SMILES: Nc1ncc(-c2cc3n(n2)CCC32CCN(C(=O)NC3(c4ccccc4Cl)CCC3)C2)cc1C(F)(F)F]

Example 376 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine-hydrochloride (75.0 mg, 208 μmol) and 1-(2-Chlorophenyl)cyclobutan-1-amine-hydrochloride (50.0 mg, 229 μmol).

1H-NMR (400 MHz, DMSO-d6) δ [ppm]: 1.649 (0.74), 1.659 (1.29), 1.670 (1.83), 1.682 (1.83), 1.686 (1.89), 1.697 (2.04), 1.708 (1.54), 1.719 (0.94), 1.730 (0.52), 2.004 (2.70), 2.017 (4.47), 2.030 (5.91), 2.047 (4.86), 2.069 (2.58), 2.074 (2.81), 2.095 (1.61), 2.116 (0.45), 2.322 (0.67), 2.326 (0.95), 2.332 (0.64), 2.434 (0.42), 2.452 (1.89), 2.467 (4.34), 2.476 (6.63), 2.518 (6.13), 2.522 (5.23), 2.539 (2.60), 2.548 (3.18), 2.569 (1.41), 2.597 (1.96), 2.608 (2.31), 2.621 (3.72), 2.632 (3.25), 2.639 (2.61), 2.644 (2.93), 2.655 (2.90), 2.664 (1.86), 2.668 (2.43), 2.678 (1.42), 3.355 (2.66), 3.365 (3.07), 3.382 (7.91), 3.393 (9.26), 3.420 (2.19), 3.451 (1.46), 3.469 (2.70), 3.484 (1.96), 3.493 (1.76), 3.509 (0.87), 4.143 (5.61), 4.161 (11.19), 4.178 (5.36), 6.344 (12.15), 6.520 (10.84), 6.554 (16.00), 7.155 (2.53), 7.159 (2.66), 7.173 (6.08), 7.178 (6.23), 7.192 (5.65), 7.197 (5.41), 7.224 (4.29), 7.228 (5.50), 7.243 (6.22), 7.247 (7.62), 7.261 (3.30), 7.265 (3.30), 7.273 (9.75), 7.276 (8.03), 7.291 (6.50), 7.295 (5.85), 7.528 (6.30), 7.532 (6.33), 7.547 (5.70), 7.551 (5.41), 7.973 (8.88), 7.978 (8.93), 8.544 (8.28), 8.548 (8.09).

### Example 377

### (rac)-2-[6-amino-5-(trifluoromethyl)pyridin-3-yl]-N-[2-(2-fluorophenyl)propan-2-yl]-6,7-dihydrospiro[pyrazolo[5,1-c][1,4]oxazine-4,3′-pyrrolidine]-1′-carboxamide

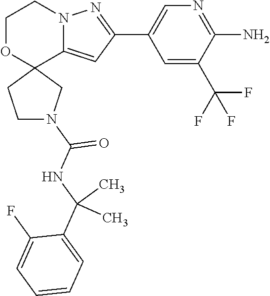 [SMILES: CC(C)(NC(=O)N1CCC2(C1)OCCn1nc(-c3cnc(N)c(C(F)(F)F)c3)cc12)c1ccccc1F]

Example 2 was prepared in analogy to Example 31 using (rac)-5-[6,7-dihydrospiro[pyrazolo[5,1-c][1,4]oxazine-4,3′-pyrrolidin]-2-yl]-3-(trifluoromethyl)pyridin-2-amine-hydrochloride and 2-(2-fluorophenyl)propan-2-amine-hydrochloride.

1H-NMR (400 MHz, DMSO-d6) δ [ppm]: 1.066 (0.63), 1.629 (15.95), 1.635 (16.00), 2.212 (0.78), 2.221 (0.87), 2.247 (1.23), 2.270 (0.66), 2.315 (1.04), 2.327 (1.43), 2.346 (0.72), 2.362 (0.61), 2.522 (1.29), 3.363 (0.72), 3.389 (1.23), 3.406 (1.24), 3.431 (0.57), 3.471 (1.84), 3.501 (2.15), 3.570 (0.94), 3.591 (1.66), 3.613 (0.75), 3.779 (1.95), 3.808 (1.66), 4.069 (0.59), 4.084 (1.25), 4.101 (1.78), 4.119 (2.96), 4.130 (5.04), 4.142 (5.33), 6.114 (5.49), 6.600 (6.89), 6.760 (10.59), 7.030 (1.27), 7.033 (1.42), 7.050 (1.64), 7.053 (1.85), 7.062 (1.32), 7.065 (1.58), 7.074 (1.64), 7.078 (1.68), 7.084 (2.16), 7.093 (3.41), 7.096 (2.79), 7.112 (2.30), 7.115 (1.95), 7.190 (0.85), 7.194 (1.02), 7.202 (1.01), 7.207 (1.42), 7.214 (1.35), 7.221 (1.32), 7.226 (1.32), 7.233 (0.70), 7.240 (0.61), 7.245 (0.55), 7.300 (1.39), 7.303 (1.38), 7.321 (2.22), 7.341 (1.22), 7.345 (1.07), 8.014 (4.23), 8.019 (4.31), 8.583 (4.00), 8.587 (3.98).

### Example 378

### (rac)-2-[6-amino-5-(trifluoromethyl)pyridin-3-yl]-N-(1-phenylcyclobutyl)-6,7-dihydrospiro[pyrazolo[5,1-c][1,4]oxazine-4,3′-pyrrolidine]-1′-carboxamide

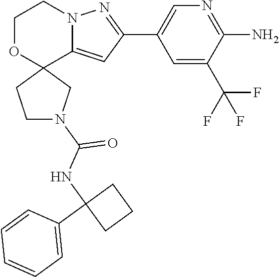 [SMILES: Nc1ncc(-c2cc3n(n2)CCOC32CCN(C(=O)NC3(c4ccccc4)CCC3)C2)cc1C(F)(F)F]

Example 3 was prepared in analogy to Example 31 using (rac)-5-[6,7-dihydrospiro[pyrazolo[5,1-c][1,4]oxazine-4,3′-pyrrolidin]-2-yl]-3-(trifluoromethyl)pyridin-2-amine and 1-phenylcyclobutan-1-amine-hydrochloride.

1H-NMR (400 MHz, DMSO-d6) δ [ppm]: 0.999 (0.75), 1.016 (0.86), 1.042 (0.43), 1.742 (0.89), 1.759 (1.68), 1.764 (1.79), 1.780 (1.79), 1.786 (2.00), 1.803 (1.25), 1.825 (0.50), 1.964 (0.86), 1.970 (1.07), 1.986 (1.90), 2.009 (1.75), 2.013 (1.68), 2.030 (1.04), 2.036 (1.00), 2.052 (0.64), 2.074 (0.64), 2.176 (0.61), 2.207 (1.36), 2.233 (1.90), 2.257 (1.00), 2.318 (2.04), 2.322 (2.86), 2.326 (3.94), 2.331 (3.15), 2.351 (1.72), 2.368 (2.29), 2.374 (2.43), 2.398 (3.65), 2.419 (2.90), 2.441 (3.54), 2.464 (4.94), 2.518 (8.88), 2.522 (5.69), 2.664 (1.54), 2.669 (2.08), 2.673 (1.54), 3.379 (2.47), 3.449 (2.40), 3.478 (2.72), 3.558 (1.61), 3.580 (2.76), 3.603 (1.29), 3.757 (3.29), 3.785 (2.83), 4.051 (1.11), 4.066 (2.04), 4.081 (2.79), 4.099 (3.22), 4.111 (3.54), 4.119 (5.87), 4.133 (10.49), 4.184 (0.79), 6.418 (1.22), 6.555 (0.97), 6.599 (12.78), 6.702 (8.20), 6.748 (16.00), 7.138 (1.54), 7.141 (2.76), 7.144 (1.83), 7.159 (6.80), 7.164 (2.68), 7.174 (2.68), 7.178 (4.65), 7.181 (2.58), 7.256 (0.61), 7.270 (7.80), 7.290 (12.42), 7.303 (2.54), 7.309 (7.19), 7.413 (10.99), 7.416 (12.74), 7.433 (9.91), 7.437 (8.09), 8.004 (7.59), 8.009 (7.55), 8.572 (7.55), 8.576 (7.41).

### Example 379

### (rac)-2′-[6-amino-5-(trifluoromethyl)pyridin-3-yl]-N-[2-(4-fluorophenyl)propan-2-yl]-5′,6′-dihydrospiro[pyrrolidine-3,4′-pyrrolo[1,2-b]pyrazole]-1-carboxamide

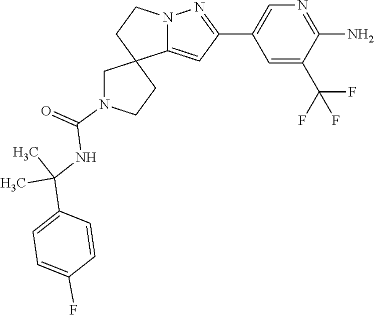 [SMILES: CC(C)(NC(=O)N1CCC2(CCn3nc(-c4cnc(N)c(C(F)(F)F)c4)cc32)C1)c1ccc(F)cc1]

Example 4 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine-hydrochloride and 2-(4-fluorophenyl)propan-2-amine.

1H-NMR (400 MHz, DMSO-d6) δ [ppm]: 1.547 (15.16), 1.556 (16.00), 2.024 (0.40), 2.038 (1.09), 2.052 (2.16), 2.069 (2.59), 2.073 (3.02), 2.086 (1.07), 2.099 (0.53), 2.518 (3.81), 2.534 (3.73), 2.539 (3.86), 2.553 (2.37), 3.394 (0.53), 3.413 (1.09), 3.420 (1.32), 3.449 (5.63), 3.476 (0.57), 3.532 (0.59), 3.549 (1.12), 3.564 (0.86), 3.575 (0.82), 3.590 (0.43), 4.175 (2.65), 4.193 (4.91), 4.210 (2.63), 6.071 (5.18), 6.456 (11.78), 6.558 (6.90), 7.020 (3.48), 7.025 (1.21), 7.042 (7.50), 7.059 (1.29), 7.064 (3.99), 7.072 (0.49), 7.351 (3.60), 7.357 (1.63), 7.365 (4.02), 7.374 (3.80), 7.382 (1.47), 7.388 (3.30), 8.008 (3.90), 8.013 (3.95), 8.148 (0.63), 8.591 (3.68), 8.596 (3.63).

### Example 380

### (rac)-2′-[6-amino-5-(trifluoromethyl)pyridin-3-yl]-N-[2-(2-chlorophenyl)propan-2-yl]-5′,6′-dihydrospiro[pyrrolidine-3,4′-pyrrolo[1,2-b]pyrazole]-1-carboxamide

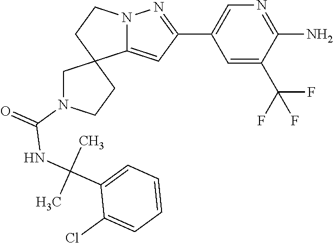 [SMILES: CC(C)(NC(=O)N1CCC2(CCn3nc(-c4cnc(N)c(C(F)(F)F)c4)cc32)C1)c1ccccc1Cl]

Example 5 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine-hydrochloride and 2-(2-chlorophenyl)propan-2-amine-hydrochloride.

¹H-NMR (400 MHz, DMSO-d6) δ [ppm]: 1.231 (0.74), 1.683 (16.00), 1.692 (15.08), 2.033 (1.15), 2.047 (2.21), 2.064 (2.62), 2.081 (1.09), 2.094 (0.51), 2.336 (0.60), 2.518 (11.25), 2.522 (6.20), 2.529 (2.70), 2.678 (0.65), 3.382 (0.71), 3.416 (8.52), 3.443 (0.79), 3.488 (0.68), 3.506 (1.27), 3.521 (0.92), 3.530 (0.82), 3.546 (0.42), 4.162 (2.58), 4.180 (5.48), 4.198 (2.51), 5.759 (8.09), 6.121 (5.58), 6.432 (11.74), 6.559 (7.21), 7.157 (1.11), 7.161 (1.12), 7.176 (2.60), 7.180 (2.73), 7.195 (2.50), 7.199 (2.38), 7.227 (1.68), 7.231 (2.24), 7.247 (2.35), 7.250 (2.99), 7.265 (1.51), 7.269 (1.51), 7.275 (4.51), 7.279 (3.76), 7.294 (3.07), 7.298 (2.67), 7.457 (2.72), 7.461 (2.81), 7.477 (2.35), 7.481 (2.26), 7.995 (4.07), 8.000 (4.11), 8.204 (0.64), 8.575 (3.81), 8.579 (3.77).

### Example 381

### 2-[6-amino-5-(trifluoromethyl)pyridin-3-yl]-N-[(1R)-1-(2-chloro-4-cyanophenyl)ethyl]-6,7-dihydrospiro[pyrazolo[5,1-c][1,4]oxazine-4,3′-pyrrolidine]-1′-carboxamide (Mixture of Stereoisomer)

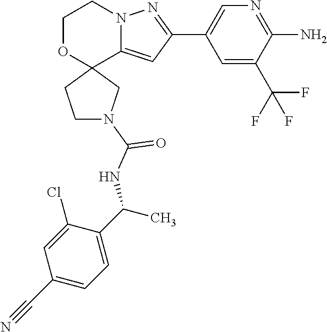 [SMILES: C[C@@H](NC(=O)N1CCC2(C1)OCCn1nc(-c3cnc(N)c(C(F)(F)F)c3)cc12)c1ccc(C#N)cc1Cl]

Example 6 was prepared in analogy to Example 31 using 5-[6,7-dihydrospiro[pyrazolo[5,1-c][1,4]oxazine-4,3′-pyrrolidin]-2-yl]-3-(trifluoromethyl)pyridin-2-amine-hydrochloride salt (75 mg, 200 μmol) and 4-[(1R)-1-aminoethyl]-3-chlorobenzonitrile (37 mg, 205 μmol).

1H-NMR (400 MHz, DMSO-d6) δ [ppm]: 0.829 (0.56), 1.052 (0.68), 1.329 (15.85), 1.347 (16.00), 2.073 (5.58), 2.202 (0.49), 2.226 (1.48), 2.254 (1.78), 2.278 (0.82), 2.322 (0.51), 2.327 (0.74), 2.332 (0.85), 2.354 (1.81), 2.518 (1.97), 2.522 (1.27), 2.669 (0.51), 3.387 (1.42), 3.404 (1.60), 3.430 (1.68), 3.449 (2.11), 3.478 (1.55), 3.514 (1.26), 3.544 (1.53), 3.559 (1.05), 3.583 (1.54), 3.606 (0.68), 3.644 (0.88), 3.666 (1.57), 3.689 (0.72), 3.773 (1.93), 3.802 (1.60), 3.850 (1.98), 3.877 (1.71), 4.052 (0.76), 4.066 (1.61), 4.082 (2.54), 4.100 (2.81), 4.112 (3.46), 4.123 (4.58), 4.132 (6.17), 4.138 (8.08), 4.144 (9.33), 5.108 (0.48), 5.120 (1.82), 5.126 (2.02), 5.138 (2.90), 5.144 (3.04), 5.156 (1.95), 5.162 (1.92), 5.172 (0.49), 6.600 (12.74), 6.760 (8.29), 6.766 (9.95), 6.784 (2.39), 6.803 (2.28), 6.822 (2.24), 6.841 (2.09), 7.661 (4.45), 7.681 (7.00), 7.700 (5.28), 7.819 (3.39), 7.823 (3.51), 7.832 (3.83), 7.836 (4.19), 7.839 (3.00), 7.843 (2.97), 7.851 (2.84), 7.856 (3.00), 7.988 (10.66), 7.991 (10.30), 8.008 (4.39), 8.014 (6.71), 8.021 (4.68), 8.136 (3.18), 8.576 (4.07), 8.581 (4.19), 8.587 (4.49), 8.592 (4.20).

### Example 382

### 2′-[6-amino-5-(trifluoromethyl)pyridin-3-yl]-N-[(1R)-1-(2-chloro-4-cyanophenyl)ethyl]-5′,6′-dihydrospiro[pyrrolidine-3,4′-pyrrolo[1,2-b]pyrazole]-1-carboxamide (Mixture of Stereoisomers)

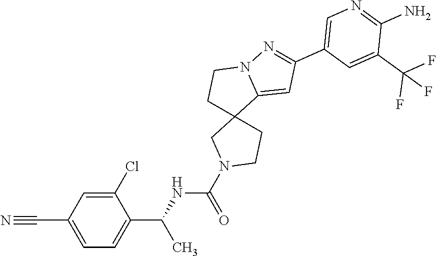 [SMILES: C[C@@H](NC(=O)N1CCC2(CCn3nc(-c4cnc(N)c(C(F)(F)F)c4)cc32)C1)c1ccc(C#N)cc1Cl]

Example 7 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine-hydrochloride and 4-[(1R)-1-aminoethyl]-3-chlorobenzonitrile.

1H-NMR (400 MHz, DMSO-d6) δ [ppm]: 1.323 (9.01), 1.330 (9.90), 1.341 (9.38), 1.347 (9.52), 2.074 (3.85), 2.083 (3.94), 2.518 (4.04), 2.523 (5.54), 2.539 (15.01), 2.558 (3.80), 3.382 (0.47), 3.429 (2.86), 3.453 (5.44), 3.483 (3.00), 3.493 (3.75), 3.518 (2.25), 3.530 (1.50), 3.546 (0.94), 3.556 (0.75), 3.574 (0.70), 3.594 (0.94), 4.172 (3.05), 4.177 (3.28), 4.190 (5.35), 4.194 (5.96), 4.207 (3.19), 4.212 (2.82), 5.111 (0.56), 5.128 (2.53), 5.146 (3.94), 5.164 (2.53), 5.181 (0.56), 6.472 (16.00), 6.544 (12.57), 6.773 (2.21), 6.789 (3.66), 6.807 (2.16), 7.642 (3.05), 7.663 (3.94), 7.705 (2.96), 7.725 (4.08), 7.775 (2.39), 7.778 (2.39), 7.795 (1.83), 7.799 (1.92), 7.808 (3.24), 7.812 (3.28), 7.828 (2.11), 7.832 (2.25), 7.980 (6.71), 7.984 (8.73), 7.989 (6.10), 8.014 (6.57), 8.133 (0.70), 8.590 (6.57), 8.595 (6.43).

### Example 383

### 2′-[6-amino-5-(trifluoromethyl)pyridin-3-yl]-N-[(1S)-1-(3-chloropyridin-4-yl)ethyl]-5′,6′-dihydrospiro[pyrrolidine-3,4′-pyrrolo[1,2-b]pyrazole]-1-carboxamide (Mixture of Stereoisomers)

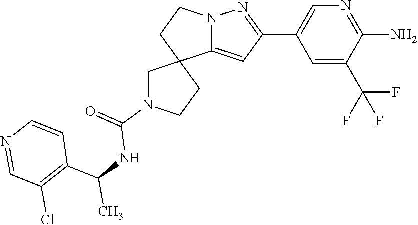 [SMILES: C[C@H](NC(=O)N1CCC2(CCn3nc(-c4cnc(N)c(C(F)(F)F)c4)cc32)C1)c1ccncc1Cl]

Example 8 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine-hydrochloride and (1S)-1-(3-chloropyridin-4-yl)ethan-1-amine-hydrochloride.

¹H-NMR (400 MHz, DMSO-d6) δ [ppm]: 1.232 (0.89), 1.332 (12.11), 1.336 (12.56), 1.350 (12.44), 1.353 (12.48), 1.902 (3.12), 2.085 (4.66), 2.323 (1.66), 2.327 (2.35), 2.332 (1.70), 2.518 (10.21), 2.523 (6.97), 2.530 (5.51), 2.548 (7.90), 2.566 (4.82), 2.660 (0.81), 2.665 (1.74), 2.669 (2.43), 2.673 (1.74), 3.420 (0.81), 3.438 (3.40), 3.464 (6.64), 3.491 (4.62), 3.520 (2.55), 3.539 (1.78), 3.555 (1.09), 3.565 (0.89), 3.583 (0.81), 3.603 (1.13), 3.627 (0.85), 4.179 (4.17), 4.195 (7.37), 4.211 (3.93), 5.036 (0.57), 5.054 (2.35), 5.072 (3.61), 5.088 (2.39), 5.106 (0.61), 6.469 (16.00), 6.552 (13.73), 6.762 (2.63), 6.779 (3.93), 6.795 (2.43), 7.454 (3.48), 7.466 (3.56), 7.532 (3.77), 7.544 (3.85), 8.014 (7.53), 8.019 (7.74), 8.375 (0.41), 8.435 (4.09), 8.448 (3.89), 8.477 (5.87), 8.490 (5.63), 8.525 (11.50), 8.528 (11.42), 8.588 (7.53), 8.593 (7.45).

### Example 384

### (rac)-2′-[6-amino-5-(trifluoromethyl)pyridin-3-yl]-N-[2-(3-fluorophenyl)propan-2-yl]-5′,6′-dihydrospiro[pyrrolidine-3,4′-pyrrolo[1,2-b]pyrazole]-1-carboxamide

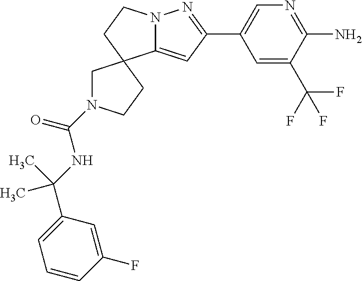 [SMILES: CC(C)(NC(=O)N1CCC2(CCn3nc(-c4cnc(N)c(C(F)(F)F)c4)cc32)C1)c1cccc(F)c1]

Example 9 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine-hydrochloride and 2-(3-fluorophenyl)propan-2-amine.

1H-NMR (400 MHz, DMSO-d6) δ [ppm]: 1.550 (14.22), 1.556 (16.00), 2.047 (1.03), 2.062 (1.97), 2.076 (2.35), 2.092 (1.00), 2.327 (0.78), 2.523 (4.68), 2.544 (3.82), 2.561 (2.43), 2.669 (0.78), 3.426 (1.55), 3.451 (3.61), 3.466 (3.18), 3.492 (0.78), 3.542 (0.54), 3.559 (1.01), 4.178 (2.35), 4.196 (4.13), 4.213 (2.35), 6.121 (4.44), 6.444 (8.64), 6.556 (6.03), 6.925 (0.79), 6.931 (0.92), 6.946 (1.50), 6.951 (1.89), 6.967 (0.88), 6.974 (0.93), 7.112 (1.64), 7.117 (1.33), 7.140 (1.63), 7.175 (1.73), 7.196 (2.52), 7.257 (1.22), 7.276 (2.11), 7.292 (1.90), 7.312 (0.76), 8.003 (3.38), 8.009 (3.60), 8.584 (3.25), 8.588 (3.36).

### Example 385

### (rac)-2-[6-amino-5-(trifluoromethyl)pyridin-3-yl]-N-[1-(pyridin-4-yl)cyclobutyl]-6,7-dihydrospiro[pyrazolo[5,1-c][1,4]oxazine-4,3′-pyrrolidine]-1′-carboxamide

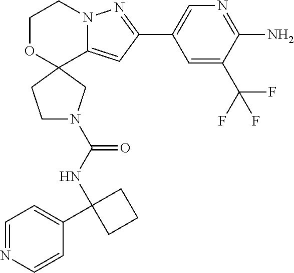 [SMILES: Nc1ncc(-c2cc3n(n2)CCOC32CCN(C(=O)NC3(c4ccncc4)CCC3)C2)cc1C(F)(F)F]

Example 10 was prepared in analogy to Example 31 using (rac)-5-[6,7-dihydrospiro[pyrazolo[5,1-c][1,4]oxazine-4,3′-pyrrolidin]-2-yl]-3-(trifluoromethyl)pyridin-2-amine-hydrochloride and 1-(pyridin-4-yl)cyclobutan-1-amine.

¹H-NMR (400 MHz, DMSO-d6) δ [ppm]: 1.009 (0.86), 1.026 (0.86), 1.231 (1.35), 1.822 (2.02), 1.843 (2.40), 1.861 (1.59), 2.007 (2.35), 2.030 (2.16), 2.074 (6.87), 2.221 (1.87), 2.245 (2.40), 2.269 (1.30), 2.327 (5.33), 2.344 (3.84), 2.387 (5.24), 2.411 (7.78), 2.430 (7.35), 2.451 (5.24), 2.669 (2.88), 3.471 (2.45), 3.498 (2.74), 3.602 (3.17), 3.770 (3.75), 3.797 (3.36), 4.062 (1.44), 4.078 (2.93), 4.093 (3.56), 4.129 (10.38), 4.141 (14.32), 6.604 (16.00), 6.765 (14.75), 6.884 (8.84), 7.039 (0.82), 7.386 (15.23), 7.390 (10.19), 7.402 (14.75), 7.638 (0.48), 8.010 (9.61), 8.014 (9.90), 8.140 (14.94), 8.205 (0.58), 8.476 (15.57), 8.480 (10.09), 8.491 (14.32), 8.577 (8.98).

### Example 386

### (rac)-2′-[6-amino-5-(trifluoromethyl)pyridin-3-yl]-N-[2-(3-chloropyridin-4-yl)propan-2-yl]-5′,6′-dihydrospiro[pyrrolidine-3,4′-pyrrolo[1,2-b]pyrazole]-1-carboxamide

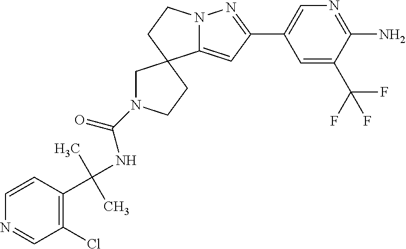 [SMILES: CC(C)(NC(=O)N1CCC2(CCn3nc(-c4cnc(N)c(C(F)(F)F)c4)cc32)C1)c1ccncc1Cl]

Example 11 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine-hydrochloride and 2-(3-chloropyridin-4-yl)propan-2-amine.

¹H-NMR (400 MHz, DMSO-d6) δ [ppm]: 1.164 (0.49), 1.232 (0.79), 1.247 (0.70), 1.257 (0.82), 1.332 (0.78), 1.560 (0.62), 1.654 (16.00), 2.036 (0.59), 2.051 (1.09), 2.067 (1.30), 2.084 (0.56), 2.322 (0.47), 2.327 (0.65), 2.332 (0.48), 2.518 (3.72), 2.522 (2.95), 2.537 (0.96), 2.664 (0.46), 2.669 (0.66), 2.673 (0.48), 2.728 (2.37), 2.888 (3.01), 3.417 (4.16), 3.440 (0.54), 3.505 (0.64), 3.521 (0.48), 3.528 (0.43), 4.165 (1.43), 4.183 (3.04), 4.200 (1.41), 5.758 (1.02), 6.332 (2.86), 6.443 (5.96), 6.558 (3.76), 7.427 (2.41), 7.440 (2.44), 8.001 (2.08), 8.006 (2.16), 8.147 (3.29), 8.389 (3.42), 8.402 (3.34), 8.408 (6.45), 8.578 (1.96), 8.581 (1.98).

### Example 387

### (rac)-2′-[6-amino-5-(trifluoromethyl)pyridin-3-yl]-N-(1-phenylcyclobutyl)-5′,6′-dihydrospiro[pyrrolidine-3,4′-pyrrolo[1,2-b]pyrazole]-1-carboxamide

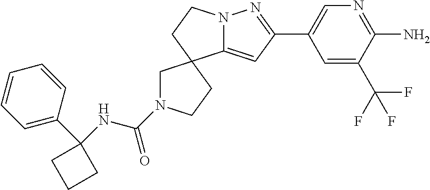 [SMILES: Nc1ncc(-c2cc3n(n2)CCC32CCN(C(=O)NC3(c4ccccc4)CCC3)C2)cc1C(F)(F)F]

Example 12 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine-hydrochloride and 1-phenylcyclobutan-1-amine-hydrochloride.

1H-NMR (400 MHz, DMSO-d6) δ [ppm]: 0.999 (0.66), 1.015 (0.69), 1.042 (0.42), 1.056 (1.13), 1.066 (0.98), 1.072 (1.13), 1.153 (3.41), 1.171 (6.70), 1.189 (3.35), 1.230 (2.11), 1.243 (2.86), 1.248 (1.82), 1.259 (3.21), 1.262 (3.13), 1.279 (2.49), 1.745 (0.76), 1.762 (1.46), 1.768 (1.56), 1.784 (1.62), 1.790 (1.80), 1.807 (1.14), 1.829 (0.44), 1.957 (0.79), 1.963 (0.94), 1.979 (1.86), 1.986 (13.20), 2.002 (1.85), 2.021 (1.54), 2.033 (2.34), 2.048 (3.61), 2.061 (4.05), 2.078 (1.98), 2.326 (0.65), 2.354 (1.36), 2.376 (2.75), 2.385 (3.45), 2.401 (4.11), 2.407 (3.48), 2.422 (2.50), 2.447 (2.01), 2.470 (3.84), 2.526 (7.40), 2.530 (6.83), 2.546 (4.36), 2.668 (0.61), 3.127 (0.43), 3.383 (0.98), 3.401 (3.15), 3.426 (8.82), 3.436 (6.98), 3.462 (1.72), 3.509 (1.57), 3.526 (2.71), 3.542 (2.34), 3.552 (2.35), 3.569 (1.92), 3.662 (3.47), 3.999 (1.02), 4.016 (2.82), 4.034 (2.80), 4.052 (1.00), 4.141 (0.47), 4.167 (4.66), 4.184 (8.94), 4.201 (4.69), 6.424 (16.00), 6.597 (1.82), 6.702 (7.56), 6.745 (0.79), 7.011 (0.86), 7.127 (2.25), 7.146 (5.62), 7.164 (3.82), 7.255 (6.46), 7.275 (10.79), 7.294 (6.17), 7.419 (9.98), 7.437 (8.42), 7.440 (6.54), 7.700 (0.55), 8.009 (6.99), 8.014 (7.10), 8.574 (6.54), 8.579 (6.46).

### Example 388

### (rac)-2-[6-amino-5-(trifluoromethyl)pyridin-3-yl]-N-[2-(pyridin-2-yl)propan-2-yl]-6,7-dihydrospiro[pyrazolo[5,1-c][1,4]oxazine-4,3′-pyrrolidine]-1′-carboxamide

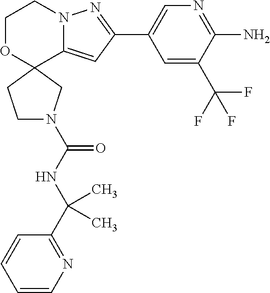 [SMILES: CC(C)(NC(=O)N1CCC2(C1)OCCn1nc(-c3cnc(N)c(C(F)(F)F)c3)cc12)c1ccccn1]

Example 13 was prepared in analogy to Example 31 using (rac)-5-[6,7-dihydrospiro[pyrazolo[5,1-c][1,4]oxazine-4,3′-pyrrolidin]-2-yl]-3-(trifluoromethyl)pyridin-2-amine-hydrochloride and 2-(pyridin-2-yl)propan-2-amine.

1H-NMR (400 MHz, DMSO-d6) δ [ppm]: 1.177 (0.43), 1.194 (0.45), 1.232 (0.58), 1.579 (16.00), 1.585 (15.80), 2.075 (1.24), 2.240 (0.63), 2.265 (0.86), 2.290 (0.45), 2.332 (1.59), 2.337 (1.21), 2.352 (0.88), 2.368 (0.48), 2.385 (0.43), 2.518 (5.38), 2.523 (3.58), 2.540 (0.66), 2.674 (1.09), 2.679 (0.48), 3.387 (0.68), 3.411 (0.96), 3.429 (0.91), 3.454 (0.45), 3.501 (1.19), 3.530 (1.41), 3.605 (0.73), 3.626 (1.31), 3.648 (0.61), 3.801 (1.59), 3.829 (1.34), 4.097 (0.86), 4.115 (1.56), 4.136 (5.15), 4.146 (3.89), 6.425 (4.06), 6.601 (5.70), 6.776 (9.44), 7.171 (1.62), 7.174 (1.72), 7.184 (1.64), 7.186 (1.89), 7.190 (1.89), 7.193 (1.74), 7.202 (1.84), 7.205 (1.84), 7.438 (1.74), 7.440 (3.08), 7.458 (2.07), 7.461 (3.33), 7.698 (1.79), 7.702 (1.74), 7.717 (2.07), 7.721 (2.12), 7.736 (1.41), 7.740 (1.31), 8.017 (3.41), 8.022 (3.46), 8.163 (2.22), 8.449 (1.84), 8.451 (2.15), 8.453 (2.25), 8.455 (1.92), 8.461 (1.92), 8.463 (2.22), 8.465 (2.07), 8.467 (1.77), 8.587 (3.18), 8.591 (3.18).

### Example 389

### (rac)-2-[6-amino-5-(trifluoromethyl)pyridin-3-yl]-N-[1-(2-fluorophenyl)cyclobutyl]-6,7-dihydrospiro[pyrazolo[5,1-c][1,4]oxazine-4,3′-pyrrolidine]-1′-carboxamide

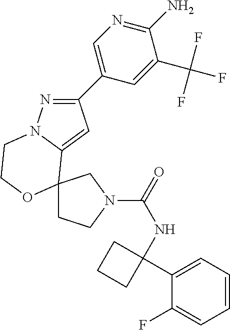 [SMILES: Nc1ncc(-c2cc3n(n2)CCOC32CCN(C(=O)NC3(c4ccccc4F)CCC3)C2)cc1C(F)(F)F]

Example 389 was prepared in analogy to Example 31 using (rac)-5-[6,7-dihydrospiro[pyrazolo[5,1-c][1,4]oxazine-4,3′-pyrrolidin]-2-yl]-3-(trifluoromethyl)pyridin-2-amine hydrochloride (100 mg, 266 μmol) and 1-(2-Fluorophenyl)cyclobutan-1-amine (52.8 mg, 319 μmol).

¹H-NMR (400 MHz, DMSO-d6) δ [ppm]: 0.917 (0.45), 0.932 (3.79), 0.934 (1.29), 0.948 (3.92), 0.952 (0.66), 1.401 (1.31), 1.425 (1.11), 1.707 (0.51), 1.714 (0.62), 1.719 (0.76), 1.727 (1.17), 1.733 (1.19), 1.741 (1.29), 1.748 (1.09), 1.755 (1.37), 1.762 (0.80), 1.768 (0.86), 1.775 (0.60), 2.014 (0.90), 2.033 (1.54), 2.055 (1.42), 2.060 (1.40), 2.074 (4.20), 2.080 (0.88), 2.156 (0.45), 2.187 (1.05), 2.213 (1.44), 2.238 (0.76), 2.292 (1.31), 2.308 (1.60), 2.323 (1.68), 2.327 (2.01), 2.332 (1.46), 2.336 (1.05), 2.405 (0.45), 2.423 (0.45), 2.454 (0.78), 2.518 (7.24), 2.523 (6.95), 2.665 (0.80), 2.669 (1.13), 2.673 (0.82), 3.370 (0.86), 3.417 (2.05), 3.445 (2.40), 3.520 (1.27), 3.542 (2.30), 3.565 (1.03), 3.732 (2.58), 3.761 (2.20), 4.030 (0.90), 4.045 (1.44), 4.061 (2.22), 4.080 (2.15), 4.092 (2.71), 4.102 (2.99), 4.122 (7.51), 6.595 (16.00), 6.729 (11.84), 7.041 (1.81), 7.044 (1.99), 7.061 (2.40), 7.064 (2.71), 7.072 (5.03), 7.091 (8.70), 7.110 (3.32), 7.113 (2.60), 7.197 (1.21), 7.201 (1.37), 7.209 (1.40), 7.214 (2.13), 7.221 (1.83), 7.228 (1.74), 7.233 (1.99), 7.239 (0.96), 7.248 (0.86), 7.252 (0.80), 7.404 (1.85), 7.409 (1.87), 7.425 (3.04), 7.444 (1.74), 7.995 (5.76), 8.000 (5.93), 8.560 (5.46), 8.564 (5.39).

### Example 390

### (rac)-2′-[6-amino-5-(trifluoromethyl)pyridin-3-yl]-N-[1-(2-fluorophenyl)cyclobutyl]-5′,6′-dihydrospiro[pyrrolidine-3,4′-pyrrolo[1,2-b]pyrazole]-1-carboxamide

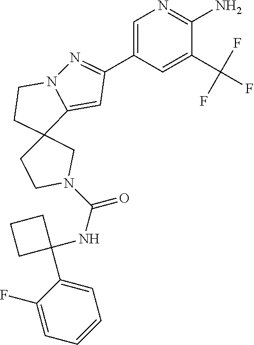 [SMILES: Nc1ncc(-c2cc3n(n2)CCC32CCN(C(=O)NC3(c4ccccc4F)CCC3)C2)cc1C(F)(F)F]

Example 390 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (50.0 mg, 139 μmol) and 1-(2-Fluorophenyl)cyclobutan-1-amine (27.6 mg, 167 μmol).

¹H-NMR (400 MHz, DMSO-d6) δ [ppm]: 0.949 (2.09), 0.965 (1.93), 1.704 (0.68), 1.712 (0.78), 1.717 (0.95), 1.725 (1.50), 1.731 (1.53), 1.739 (1.61), 1.745 (1.40), 1.753 (1.94), 1.766 (1.09), 1.774 (0.79), 1.787 (0.42), 2.009 (3.17), 2.026 (5.12), 2.036 (5.21), 2.049 (3.24), 2.054 (3.63), 2.068 (1.57), 2.084 (0.62), 2.094 (0.46), 2.323 (0.56), 2.327 (0.78), 2.332 (0.58), 2.457 (1.19), 2.518 (8.72), 2.524 (10.01), 2.547 (3.28), 2.576 (0.82), 2.665 (0.60), 2.669 (0.82), 2.673 (0.60), 3.368 (3.24), 3.397 (10.10), 3.403 (9.09), 3.429 (1.35), 3.470 (1.27), 3.488 (2.33), 3.496 (1.54), 3.502 (1.70), 3.514 (1.54), 3.529 (0.83), 4.151 (5.04), 4.169 (10.49), 4.186 (4.86), 6.389 (16.00), 6.549 (13.29), 6.582 (8.78), 7.025 (2.30), 7.027 (2.59), 7.045 (2.99), 7.048 (3.41), 7.054 (2.45), 7.057 (2.82), 7.064 (3.24), 7.067 (2.99), 7.077 (3.83), 7.083 (7.41), 7.086 (5.55), 7.102 (4.27), 7.105 (3.41), 7.182 (1.58), 7.186 (1.81), 7.195 (1.84), 7.200 (2.73), 7.206 (2.30), 7.214 (2.33), 7.219 (2.42), 7.225 (1.21), 7.233 (1.12), 7.238 (1.02), 7.407 (2.45), 7.412 (2.47), 7.428 (3.84), 7.431 (3.65), 7.448 (2.22), 7.452 (1.94), 7.984 (7.45), 7.989 (7.57), 8.556 (6.98), 8.561 (6.86).

### Example 391

### (rac)-2′-[6-amino-5-(trifluoromethyl)pyridin-3-yl]-N-[1-(3-fluorophenyl)cyclobutyl]-5′,6′-dihydrospiro[pyrrolidine-3,4′-pyrrolo[1,2-b]pyrazole]-1-carboxamide

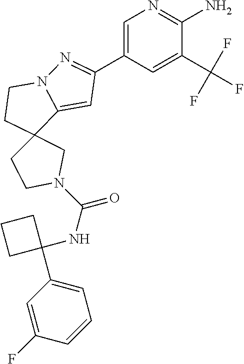 [SMILES: Nc1ncc(-c2cc3n(n2)CCC32CCN(C(=O)NC3(c4cccc(F)c4)CCC3)C2)cc1C(F)(F)F]

Example 391 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (50.0 mg, 139 μmol) and 1-(3-Fluorophenyl)cyclobutan-1-amine hydrochloride (33.6 mg, 167 μmol).

¹H-NMR (400 MHz, DMSO-d6) δ [ppm]: 0.945 (2.14), 0.961 (2.04), 1.753 (0.63), 1.760 (0.62), 1.776 (1.24), 1.781 (1.28), 1.798 (1.32), 1.803 (1.53), 1.820 (0.93), 1.959 (0.60), 1.966 (0.77), 1.981 (1.45), 1.997 (1.07), 2.004 (1.38), 2.009 (1.43), 2.024 (1.18), 2.032 (1.18), 2.041 (1.77), 2.055 (2.85), 2.068 (3.17), 2.084 (1.64), 2.097 (0.59), 2.323 (0.58), 2.327 (0.81), 2.332 (0.60), 2.348 (0.98), 2.372 (2.15), 2.380 (2.68), 2.388 (2.09), 2.396 (3.24), 2.402 (2.73), 2.418 (2.12), 2.430 (1.87), 2.453 (3.13), 2.460 (2.26), 2.470 (2.50), 2.476 (2.49), 2.518 (5.46), 2.523 (3.29), 2.532 (5.37), 2.536 (5.37), 2.551 (3.64), 2.665 (0.60), 2.669 (0.84), 2.674 (0.58), 3.389 (0.88), 3.406 (2.63), 3.431 (6.90), 3.442 (5.07), 3.468 (1.18), 3.516 (0.88), 3.533 (1.69), 3.548 (1.22), 3.558 (1.21), 3.574 (0.60), 4.169 (4.12), 4.187 (7.49), 4.204 (3.99), 6.421 (16.00), 6.551 (10.69), 6.763 (7.02), 6.949 (1.18), 6.952 (1.28), 6.955 (1.49), 6.958 (1.47), 6.971 (2.56), 6.975 (2.46), 6.978 (2.78), 6.991 (1.39), 6.993 (1.49), 6.998 (1.62), 7.000 (1.55), 7.163 (2.04), 7.169 (2.63), 7.173 (2.23), 7.190 (2.12), 7.195 (2.67), 7.200 (2.16), 7.259 (2.58), 7.279 (5.25), 7.298 (3.05), 7.313 (3.16), 7.317 (3.96), 7.333 (3.82), 7.337 (1.62), 7.352 (1.45), 7.994 (6.07), 7.999 (6.17), 8.570 (5.65), 8.575 (5.62).

### Example 392

### (rac)-2′-[6-amino-5-(trifluoromethyl)pyridin-3-yl]-N-[1-(pyridin-4-yl)cyclobutyl]-5′,6′-dihydrospiro[pyrrolidine-3,4′-pyrrolo[1,2-b]pyrazole]-1-carboxamide

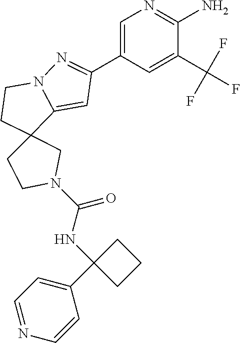 [SMILES: Nc1ncc(-c2cc3n(n2)CCC32CCN(C(=O)NC3(c4ccncc4)CCC3)C2)cc1C(F)(F)F]

Example 392 was prepared in analogy to Example 31 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (50.0 mg, 139 μmol) and 1-(Pyridin-4-yl)cyclobutan-1-amine hydrochloride (36.9 mg, 167 μmol).

¹H-NMR (400 MHz, DMSO-d6) δ [ppm]: 0.840 (0.44), 0.858 (0.44), 0.919 (0.61), 0.934 (4.85), 0.950 (4.81), 1.237 (0.44), 1.803 (0.78), 1.825 (1.59), 1.848 (2.00), 1.866 (1.22), 1.888 (0.54), 2.000 (1.76), 2.023 (1.80), 2.050 (2.81), 2.065 (3.93), 2.078 (4.07), 2.323 (1.46), 2.327 (2.07), 2.332 (1.49), 2.344 (1.36), 2.375 (3.56), 2.391 (4.34), 2.413 (4.00), 2.444 (3.76), 2.518 (10.68), 2.523 (9.05), 2.537 (6.64), 2.543 (6.54), 2.558 (4.00), 2.665 (1.49), 2.669 (2.03), 2.673 (1.46), 3.445 (10.37), 3.549 (2.00), 4.173 (4.92), 4.190 (9.39), 4.208 (4.75), 6.458 (16.00), 6.556 (13.08), 6.872 (8.03), 7.390 (12.75), 7.395 (8.24), 7.402 (8.41), 7.406 (12.61), 8.009 (7.36), 8.014 (7.32), 8.458 (14.58), 8.461 (8.71), 8.469 (8.61), 8.473 (13.69), 8.584 (6.98), 8.588 (6.78).

### Example 393

### (rac)-2-[6-amino-5-(trifluoromethyl)pyridin-3-yl]-N-[1-(3-fluorophenyl)cyclobutyl]-6,7-dihydrospiro[pyrazolo[5,1-c][1,4]oxazine-4,3′-pyrrolidine]-1′-carboxamide

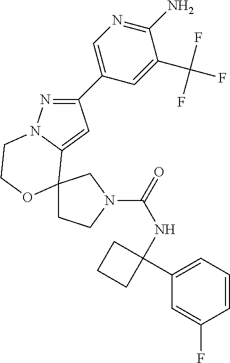 [SMILES: Nc1ncc(-c2cc3n(n2)CCOC32CCN(C(=O)NC3(c4cccc(F)c4)CCC3)C2)cc1C(F)(F)F]

Example 393 was prepared in analogy to Example 31 using (rac)-5-[6,7-dihydrospiro[pyrazolo[5,1-c][1,4]oxazine-4,3′-pyrrolidin]-2-yl]-3-(trifluoromethyl)pyridin-2-amine hydrochloride (100 mg, 266 μmol) and 1-(3-Fluorophenyl)cyclobutan-1-amine hydrochloride (64.4 mg, 319 μmol).

¹H-NMR (400 MHz, DMSO-d6) δ [ppm]: 0.917 (1.89), 0.931 (16.00), 0.934 (5.08), 0.948 (15.70), 0.952 (2.62), 1.004 (0.61), 1.021 (0.61), 1.401 (1.10), 1.425 (0.91), 1.753 (0.85), 1.759 (0.67), 1.770 (1.43), 1.776 (1.55), 1.787 (1.13), 1.793 (1.55), 1.798 (1.79), 1.809 (0.76), 1.815 (1.16), 1.836 (0.46), 1.966 (0.73), 1.971 (0.94), 1.988 (1.67), 1.993 (1.34), 2.004 (1.16), 2.010 (1.55), 2.015 (1.49), 2.026 (0.82), 2.031 (0.79), 2.038 (0.79), 2.182 (0.55), 2.214 (1.25), 2.240 (1.67), 2.264 (0.88), 2.322 (2.34), 2.327 (2.46), 2.332 (2.46), 2.336 (2.31), 2.347 (1.73), 2.358 (1.83), 2.364 (2.28), 2.370 (2.65), 2.380 (2.89), 2.387 (3.25), 2.396 (3.22), 2.404 (4.59), 2.410 (2.83), 2.422 (5.05), 2.440 (4.08), 2.446 (4.29), 2.463 (3.25), 2.471 (2.68), 2.478 (2.68), 2.518 (5.08), 2.523 (3.22), 2.665 (0.85), 2.669 (1.19), 2.673 (0.88), 2.940 (1.00), 2.957 (1.31), 2.973 (0.97), 3.370 (1.73), 3.389 (1.64), 3.413 (0.79), 3.456 (1.79), 3.485 (2.07), 3.562 (1.37), 3.584 (2.40), 3.608 (1.10), 3.762 (2.98), 3.791 (2.49), 4.056 (0.94), 4.070 (1.86), 4.086 (2.68), 4.112 (3.68), 4.121 (5.78), 4.135 (9.46), 6.598 (12.14), 6.753 (14.14), 6.769 (7.39), 6.965 (1.46), 6.967 (1.61), 6.971 (1.86), 6.974 (1.79), 6.987 (3.10), 6.993 (3.44), 7.007 (1.67), 7.009 (1.73), 7.014 (1.92), 7.015 (1.76), 7.164 (2.43), 7.169 (3.13), 7.174 (2.68), 7.191 (2.46), 7.195 (3.25), 7.201 (2.52), 7.253 (3.44), 7.273 (5.54), 7.311 (3.29), 7.326 (3.47), 7.331 (4.65), 7.347 (4.41), 7.350 (2.13), 7.366 (1.83), 8.004 (7.03), 8.009 (7.15), 8.543 (0.49), 8.572 (6.66), 8.576 (6.57).

### Example 394

### (3′R)-2-[6-amino-5-(trifluoromethoxy)pyridin-3-yl]-N-[2-(pyridin-2-yl)propan-2-yl]-6,7-dihydrospiro[pyrazolo[5,1-c][1,4]oxazine-4,3′-pyrrolidine]-1′-carboxamide

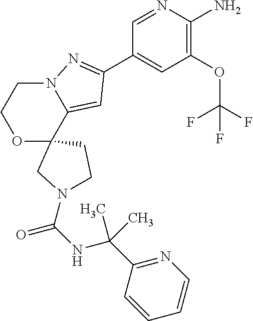 [SMILES: CC(C)(NC(=O)N1CC[C@]2(C1)OCCn1nc(-c3cnc(N)c(OC(F)(F)F)c3)cc12)c1ccccn1]

A solution of 2-(pyridin-2-yl)propan-2-amine (50.0 mg, 367 μmol), CDI (64.9 mg, 400 μmol) and DIPEA (230 μl, 1.3 mmol) in DMF (2 mL) was stirred at RT for 1 h. 5-[(3′R)-6,7-dihydrospiro[pyrazolo[5,1-c][1,4]oxazine-4,3′-pyrrolidin]-2-yl]-3-(trifluoromethoxy)pyridin-2-amine hydrogen chloride (218 mg, 60% purity, 334 μmol) in DMF (1.5 mL) was added and the mixture was stirred at RT overnight. The crude was directly purified by basic HPLC to give 14.0 mg (99% purity, 8% yield) of the title compound.

1H NMR (DMSO-d6, 400 MHz) δ 8.4-8.5 (m, 1H), 8.33 (d, 1H), 7.7-7.8 (m, 2H), 7.4-7.5 (m, 1H), 7.19 (ddd, 1H), 6.73 (s, 1H), 6.56 (s, 2H), 6.42 (s, 1H), 4.1-4.2 (m, 4H), 3.81 (d, 1H), 3.62 (br t, 1H), 3.51 (br d, 1H), 3.4-3.5 (m, 1H), 2.2-2.3 (m, 1H), 1.58 (d, 6H)

LC-MS (method 1): Rₜ=1.00 min; MS (ESIneg): m/z=516 [M−H]⁻

### Example 395

### (3′R)-2-[6-amino-5-(trifluoromethyl)pyridin-3-yl]-N-[2-(pyridin-3-yl)propan-2-yl]-6,7-dihydrospiro[pyrazolo[5,1-c][1,4]oxazine-4,3′-pyrrolidine]-1′-carboxamide

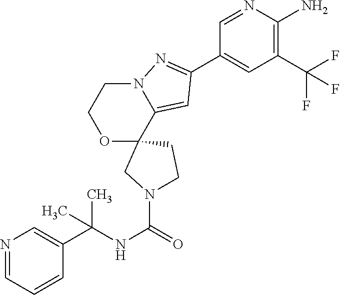 [SMILES: CC(C)(NC(=O)N1CC[C@]2(C1)OCCn1nc(-c3cnc(N)c(C(F)(F)F)c3)cc12)c1cccnc1]

A solution of 2-(pyridin-3-yl)propan-2-amine (51.0 mg, 375 μmol), CDI (66.3 mg, 409 μmol) and DIPEA (240 μl, 1.4 mmol) in DMF (2 mL) was stirred at RT for 1 h. 5-[(3′R)-6,7-dihydrospiro[pyrazolo[5,1-c][1,4]oxazine-4,3′-pyrrolidin]-2-yl]-3-(trifluoromethyl)pyridin-2-amine hydrogen chloride (160 mg, 80% purity, 341 μmol) in DMF (1.5 mL) was added and the mixture was stirred at RT overnight. The crude reaction mixture was sent to the HPLC-Lab for purification to give 18.0 mg (95% purity, 10% yield) of the title compound.

1H NMR (DMSO-d6, 400 MHz) δ 8.5-8.6 (m, 2H), 8.36 (dd, 1H), 8.02 (d, 1H), 7.7-7.7 (m, 1H), 7.29 (ddd, 1H), 6.77 (s, 1H), 6.6-6.6 (m, 2H), 6.21 (s, 1H), 4.0-4.2 (m, 5H), 3.80 (br d, 1H), 3.61 (br t, 1H), 3.50 (br d, 1H), 3.40 (br d, 1H), 2.3-2.4 (m, 1H), 2.2-2.3 (m, 1H), 1.58 (s, 6H)

LC-MS (method 1): Rₜ=0.94 min; MS (ESIpos): m/z=503 [M+H]⁺

### Example 396

### (3R)-2′-[6-amino-5-(trifluoromethyl)pyridin-3-yl]-N-[2-(pyridin-4-yl)propan-2-yl]-5′,6′-dihydrospiro[pyrrolidine-3,4′-pyrrolo[1,2-b]pyrazole]-1-carboxamide

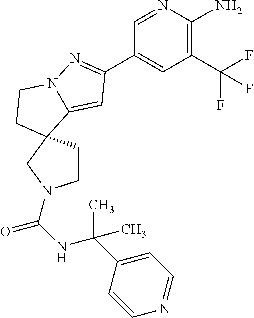 [SMILES: CC(C)(NC(=O)N1CC[C@@]2(CCn3nc(-c4cnc(N)c(C(F)(F)F)c4)cc32)C1)c1ccncc1]

A solution of 2-(pyridin-4-yl)propan-2-amine (51.0 mg, 374 μmol), CDI (66.2 mg, 408 μmol) and DIPEA (240 μl, 1.4 mmol) in DMF (2 mL) was stirred at RT for 1 h. 5-[(3R)-5′,6′-dihydrospiro[pyrrolidine-3,4′-pyrrolo[1,2-b]pyrazol]-2′-yl]-3-(trifluoromethyl)pyridin-2-amine hydrogen chloride (157 mg, 78% purity, 340 μmol) in DMF (1.5 mL) was added and the mixture was stirred at RT overnight. The crude reaction mixture was sent to the HPLC-Lab for purification to give 37.0 mg (90% purity, 20% yield) of the title compound.

1H NMR (DMSO-d6, 400 MHz) δ 8.60 (d, 1H), 8.4-8.5 (m, 2H), 8.02 (d, 1H), 7.4-7.5 (m, 2H), 6.56 (s, 2H), 6.48 (s, 1H), 6.29 (s, 1H), 4.20 (t, 2H), 3.5-3.6 (m, 1H), 3.4-3.5 (m, 3H), 2.5-2.6 (m, 2H), 2.0-2.2 (m, 2H), 1.55 (s, 6H)

LC-MS (method 1): Rₜ=0.94 min; MS (ESIpos): m/z=487 [M+H]⁺

### Example 397

### (3′R)-2-[6-amino-5-(trifluoromethyl)pyridin-3-yl]-N-[2-(pyridin-4-yl)propan-2-yl]-6,7-dihydrospiro[pyrazolo[5,1-c][1,4]oxazine-4,3′-pyrrolidine]-1′-carboxamide

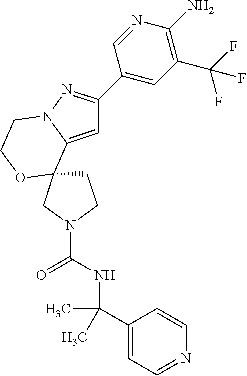 [SMILES: CC(C)(NC(=O)N1CC[C@]2(C1)OCCn1nc(-c3cnc(N)c(C(F)(F)F)c3)cc12)c1ccncc1]

A solution of 2-(pyridin-4-yl)propan-2-amine (51.0 mg, 375 μmol), CDI (66.3 mg, 409 μmol) and DIPEA (240 μl, 1.4 mmol) in DMF (2 mL) was stirred at RT for 1 h. 5-[(3′R)-6,7-dihydrospiro[pyrazolo[5,1-c][1,4]oxazine-4,3′-pyrrolidin]-2-yl]-3-(trifluoromethyl)pyridin-2-amine hydrogen chloride (160 mg, 80% purity, 341 μmol) in DMF (1.5 mL) was added and the mixture was stirred at RT overnight. The crude reaction mixture was sent to the HPLC-Lab for purification to give 71.0 mg (99% purity, 41% yield) of the title compound.

1H NMR (DMSO-d6, 400 MHz) δ 8.59 (d, 1H), 8.4-8.5 (m, 2H), 8.02 (d, 1H), 7.3-7.4 (m, 2H), 6.77 (s, 1H), 6.60 (s, 2H), 6.25 (s, 1H), 4.1-4.2 (m, 4H), 3.80 (br d, 1H), 3.6-3.7 (m, 1H), 3.50 (br d, 1H), 3.4-3.5 (m, 1H), 2.3-2.4 (m, 1H), 2.2-2.3 (m, 1H), 1.54 (s, 6H)

LC-MS (method 1): Rₜ=0.94 min; MS (ESIpos): m/z=503 [M+H]⁺

### Example 398

### (3R)-2′-[6-amino-5-(trifluoromethyl)pyridin-3-yl]-N-[2-(pyridin-3-yl)propan-2-yl]-5′,6′-dihydrospiro[pyrrolidine-3,4′-pyrrolo[1,2-b]pyrazole]-1-carboxamide

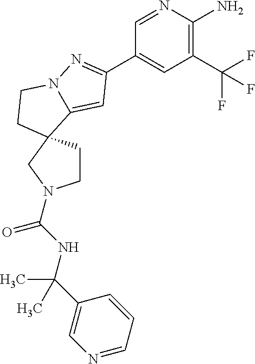 [SMILES: CC(C)(NC(=O)N1CC[C@@]2(CCn3nc(-c4cnc(N)c(C(F)(F)F)c4)cc32)C1)c1cccnc1]

A solution of 2-(pyridin-3-yl)propan-2-amine (51.0 mg, 374 μmol), CDI (66.2 mg, 408 μmol) and DIPEA (240 μl, 1.4 mmol) in DMF (2 mL) was stirred at RT for 1 h. 5-[(3R)-5′,6′-dihydrospiro[pyrrolidine-3,4′-pyrrolo[1,2-b]pyrazol]-2′-yl]-3-(trifluoromethyl)pyridin-2-amine hydrogen chloride (157 mg, 78% purity, 340 μmol) in DMF (1.5 mL) was added and the mixture was stirred at RT overnight. The crude reaction mixture was sent to the HPLC-Lab for purification to give 8.00 mg (99% purity, 5% yield) of the title compound.

1H NMR (DMSO-d6, 400 MHz) δ 8.6-8.6 (m, 2H), 8.35 (dd, 1H), 8.02 (d, 1H), 7.7-7.8 (m, 1H), 7.27 (ddd, 1H), 6.55 (s, 2H), 6.45 (s, 1H), 6.19 (s, 1H), 4.19 (t, 2H), 3.57 (td, 1H), 3.4-3.5 (m, 3H), 2.5-2.6 (m, 2H), 2.0-2.1 (m, 2H), 1.58 (d, 6H)

LC-MS (method 1): Rₜ=0.95 min; MS (ESIpos): m/z=487 [M+H]⁺

### Example 399

### (3R)-2′-[6-amino-5-(difluoromethoxy)pyridin-3-yl]-N-[(1R)-1-phenylethyl]-5′,6′-dihydrospiro[pyrrolidine-3,4′-pyrrolo[1,2-b]pyrazole]-1-carboxamide

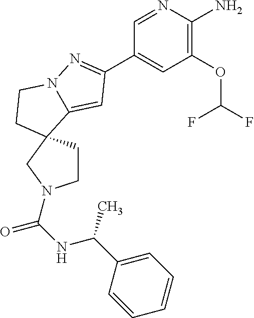 [SMILES: C[C@@H](NC(=O)N1CC[C@@]2(CCn3nc(-c4cnc(N)c(OC(F)F)c4)cc32)C1)c1ccccc1]

A solution of (1R)-1-phenylethan-1-amine (58.3 mg, 481 μmol), CDI (85.1 mg, 525 μmol) and DIPEA (300 μl, 1.7 mmol) in DMF (2 mL) was stirred at RT for 1 h. 3-(difluoromethoxy)-5-[(3R)-5′,6′-dihydrospiro[pyrrolidine-3,4′-pyrrolo[1,2-b]pyrazol]-2′-yl]pyridin-2-amine hydrogen chloride (170 mg, 92% purity, 437 μmol) in DMF (2 mL) was added and the mixture was stirred at RT overnight. The mixture was purified by basic HPLC to give 6.00 mg (95% purity, 3% yield) of the title compound.

1H NMR (DMSO-d6, 400 MHz) δ 8.20 (d, 1H), 7.6-7.6 (m, 1H), 7.37 (s, 0.25H), 7.3-7.3 (m, 2H), 7.2-7.3 (m, 2H), 7.1-7.2 (m, 1H), 7.18 (s, 0.5H), 7.00 (s, 0.25H), 6.47 (d, 1H), 6.36 (s, 1H), 6.17 (s, 2H), 4.84 (t, 1H), 4.17 (t, 2H), 3.5-3.6 (m, 1H), 3.4-3.5 (m, 3H), 2.5-2.6 (m, 2H), 2.0-2.1 (m, 2H), 1.37 (d, 3H)

LC-MS (method 1): Rₜ=1.01 min; MS (ESIpos): m/z=469 [M+H]⁺

### Example 400

### (3′R)-2-[6-amino-5-(difluoromethoxy)pyridin-3-yl]-N-[(1R)-1-phenylethyl]-6,7-dihydrospiro[pyrazolo[5,1-c][1,4]oxazine-4,3′-pyrrolidine]-1′-carboxamide

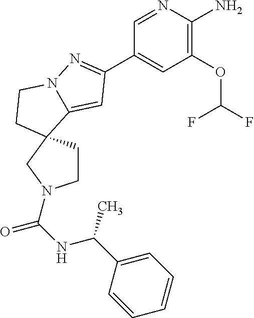 [SMILES: C[C@@H](NC(=O)N1CC[C@@]2(CCn3nc(-c4cnc(N)c(OC(F)F)c4)cc32)C1)c1ccccc1]

A solution of (1R)-1-phenylethan-1-amine (55.3 mg, 456 μmol), CDI (80.7 mg, 498 μmol) and DIPEA (290 μl, 1.7 mmol) in DMF (2 mL) was stirred at RT for 1 h. 3-(difluoromethoxy)-5-[(3′R)-6,7-dihydrospiro[pyrazolo[5,1-c][1,4]oxazine-4,3′-pyrrolidin]-2-yl]pyridin-2-amine hydrogen chloride (155 mg, 415 μmol) in DMF (2.5 mL) was added and the mixture was stirred at RT overnight. The crude was directly purified by basic HPLC to give 21.0 mg (99% purity, 10% yield) of the title compound.

1H NMR (DMSO-d6, 400 MHz) δ 8.20 (d, 1H), 7.6-7.6 (m, 1H), 7.36 (s, 0.25H), 7.3-7.4 (m, 2H), 7.3-7.3 (m, 2H), 7.2-7.2 (m, 1H), 7.18 (s, 0.5H), 7.00 (s, 0.25H), 6.67 (s, 1H), 6.5-6.5 (m, 1H), 6.22 (s, 2H), 4.84 (quin, 1H), 4.0-4.2 (m, 4H), 3.8-3.9 (m, 1H), 3.6-3.7 (m, 1H), 3.49 (d, 1H), 3.4-3.4 (m, 1H), 2.2-2.3 (m, 1H), 1.37 (d, 3H)

LC-MS (method 1): Rₜ=1.01 min; MS (ESIpos): m/z=486 [M+H]⁺

### Example 401

### (3R)-2′-[6-amino-5-(trifluoromethyl)pyridin-3-yl]-N-[2-(pyridin-2-yl)propan-2-yl]-5′,6′-dihydrospiro[pyrrolidine-3,4′-pyrrolo[1,2-b]pyrazole]-1-carboxamide

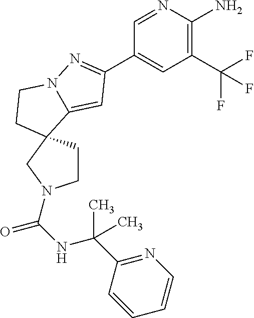 [SMILES: CC(C)(NC(=O)N1CC[C@@]2(CCn3nc(-c4cnc(N)c(C(F)(F)F)c4)cc32)C1)c1ccccn1]

A solution of 2-(pyridin-2-yl)propan-2-amine (42.8 mg, 314 μmol), CDI (55.5 mg, 343 μmol) and DIPEA (200 μl, 1.1 mmol) in DMF (2 mL) was stirred at RT for 1 h. 5-[(3R)-5′,6′-dihydrospiro[pyrrolidine-3,4′-pyrrolo[1,2-b]pyrazol]-2′-yl]-3-(trifluoromethyl)pyridin-2-amine hydrogen chloride (130 mg, 79% purity, 285 μmol) in DMF (1 mL) was added and the mixture was stirred at RT overnight. The crude was directly purified by basic HPLC to give 41.0 mg (99% purity, 29% yield) of the title compound.

1H NMR (DMSO-d6, 400 MHz) δ 8.60 (d, 1H), 8.4-8.5 (m, 1H), 8.02 (d, 1H), 7.70 (dt, 1H), 7.4-7.5 (m, 1H), 7.18 (ddd, 1H), 6.55 (s, 2H), 6.49 (s, 1H), 6.43 (s, 1H), 4.20 (t, 2H), 3.6-3.6 (m, 1H), 3.4-3.5 (m, 3H), 2.5-2.6 (m, 2H), 2.0-2.2 (m, 2H), 1.59 (s, 6H)

LC-MS (method 1): Rₜ=1.02 min; MS (ESIpos): m/z=487 [M+H]⁺

### Example 402

### 2′-[6-amino-5-(trifluoromethyl)pyridin-3-yl]-N-[(1R)-1-phenylethyl]-5′,6′-dihydrospiro[pyrrolidine-3,4′-pyrrolo[1,2-b]pyrazole]-1-carboxamide (Stereoisomer 1)

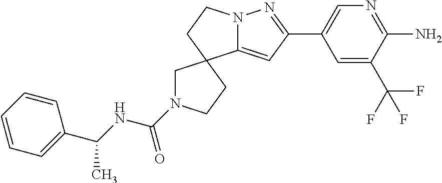 [SMILES: C[C@@H](NC(=O)N1CCC2(CCn3nc(-c4cnc(N)c(C(F)(F)F)c4)cc32)C1)c1ccccc1]

The mixture of isomers was separated by chiral HPLC to give 12.0 mg (98% purity, 12% yield) of the title compound.

Instrument: PrepCon Labomatic HPLC-3; Column: YMC Cellulose SB 10μ, 250×50; eluent A: methyl tert-butyl ether+0.1 vol % diethylamine; eluent B: acetonitrile+0.1 vol % diethylamine; isocratic: 60% A+40% B; flow: 80 mL/min; temperature: 25° C.; UV: 254 nm

Retention time: 7.2-8.1 min

Instrument: Waters Alliance 2695; Column: YMC Cellulose SB 3μ, 100×4.6; eluent A: methyl tert-butyl ether+0.1 vol % diethylamine; eluent B: acetonitrile; isocratic: 60% A+40% B; flow: 1.4 mL/min; temperature: 25° C.; UV: 254 nm

¹H NMR (400 MHz, DMSO-d₆) δ ppm 1.36 (d, 3H) 2.02-2.14 (m, 2H) 2.52-2.59 (m, 2H) 3.40-3.50 (m, 3H) 3.57 (dt, 1H) 4.19 (t, 2H) 4.84 (quin, 1H) 6.43-6.49 (m, 2H) 6.55 (s, 2H) 7.15-7.22 (m, 1H) 7.26-7.32 (m, 2H) 7.33-7.39 (m, 2H) 8.01 (d, 1H) 8.58 (d, 1H)

### Example 403

### 2′-[6-amino-5-(trifluoromethyl)pyridin-3-yl]-N-[(1R)-1-phenylethyl]-5′,6′-dihydrospiro[pyrrolidine-3,4′-pyrrolo[1,2-b]pyrazole]-1-carboxamide (Stereoisomer 2)

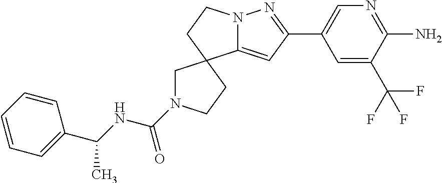 [SMILES: C[C@@H](NC(=O)N1CCC2(CCn3nc(-c4cnc(N)c(C(F)(F)F)c4)cc32)C1)c1ccccc1]

The mixture of isomers was separated by chiral HPLC to give 9.00 mg (95% purity, 9% yield) of the title compound.

Instrument: PrepCon Labomatic HPLC-3; Column: YMC Cellulose SB 10μ, 250×50; eluent A: methyl tert-butyl ether+0.1 vol % diethylamine; eluent B: acetonitrile+0.1 vol % diethylamine; isocratic: 60% A+40% B; flow: 80 mL/min; temperature: 25° C.; UV: 254 nm

Retention time: 8.3-9.3 min

Instrument: Waters Alliance 2695; Column: YMC Cellulose SB 3μ, 100×4.6; eluent A: methyl tert-butyl ether+0.1 vol % diethylamine; eluent B: acetonitrile; isocratic: 60% A+40% B; flow: 1.4 mL/min; temperature: 25° C.; UV: 254 nm

¹H-NMR (400 MHz, DMSO-d₆): δ [ppm]=1.37 (d, 3H), 2.01-2.11 (m, 2H), 2.52-2.57 (m, 2H), 3.39-3.50 (m, 3H), 3.50-3.60 (m, 1H), 4.18 (t, 2H), 4.84 (quin, 1H), 6.43-6.50 (m, 2H), 6.55 (s, 2H), 7.14-7.21 (m, 1H), 7.24-7.30 (m, 2H), 7.30-7.35 (m, 2H), 8.01 (d, 1H), 8.59 (d, 1H).

### Example 404

### (rac)-2′-[6-amino-5-(trifluoromethyl)pyridin-3-yl]-N-[2-(3,5-difluorophenyl)propan-2-yl]-5′,6′-dihydrospiro[pyrrolidine-3,4′-pyrrolo[1,2-b]pyrazole]-1-carboxamide

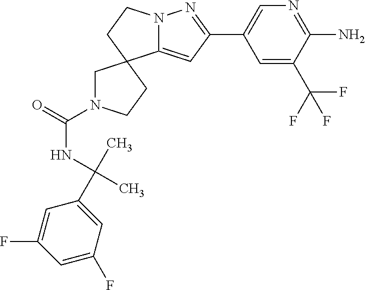 [SMILES: CC(C)(NC(=O)N1CCC2(CCn3nc(-c4cnc(N)c(C(F)(F)F)c4)cc32)C1)c1cc(F)cc(F)c1]

(rac)-5-[5′,6′-dihydrospiro[pyrrolidine-3,4′-pyrrolo[1,2-b]pyrazol]-2′-yl]-3-(trifluoromethyl)pyridin-2-amine hydrochloride (100 mg, 254 μmol) and N,N-diisopropylethylamine (130 μL) were stirred in the reaction mixture of 1,3-difluoro-5-(2-isocyanatopropan-2-yl)benzene (60.0 mg, 304 μmol) in 1,4-dioxane (2.6 mL) over the weekend at rt. NaOH (0.1 M) was added and the mixture was stirred for 5 min. The layers were separated and the aq. layer was extracted 4× with DCM. The combined organic layers were washed with NaOH (0.1 M), the aq. layer was extracted with DCM and the organic layer was evaporated. The residue was purified by flash chromatography to give 25.0 mg (90% purity, 17% yield) of the title compound.

LC-MS (method 1): Rt=1.19 min; MS (ESIpos): m/z=521 [M+H]⁺

¹H NMR (400 MHz, DMSO-d₆) δ ppm 1.54 (d, 6H) 1.99-2.13 (m, 2H) 2.54 (t, 2H) 3.41-3.52 (m, 3H) 3.53-3.64 (m, 1H) 4.20 (t, 2H) 6.17 (s, 1H) 6.44 (s, 1H) 6.55 (s, 2H) 6.94-7.05 (m, 3H) 8.00 (d, 1H) 8.58 (d, 1H)

### Example 405

### (rac)-1-{2′-[6-amino-5-(trifluoromethyl)pyridin-3-yl]-5′,6′-dihydrospiro[pyrrolidine-3,4′-pyrrolo[1,2-b]pyrazol]-1-yl}-2-(5-fluoropyridin-2-yl)-2-methylpropan-1-one

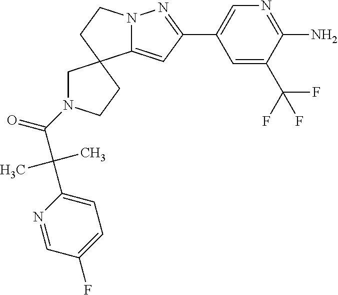 [SMILES: CC(C)(C(=O)N1CCC2(CCn3nc(-c4cnc(N)c(C(F)(F)F)c4)cc32)C1)c1ccc(F)cn1]

Example 405 was prepared in analogy to Example 404 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (110 mg, 277 μmol) and 5-fluoro-2-(2-isocyanatopropan-2-yl)pyridine (60.0 mg, 333 μmol).

LC-MS (Method 1): Rt=1.07 min; MS (ESIpos): m/z=489 [M+H]⁺

¹H NMR (400 MHz, DMSO-d₆) δ ppm 1.86 (br s, 1H) 1.92 (br s, 2H) 2.07-2.22 (m, 1H) 2.29-2.42 (m, 2H) 2.62-2.71 (m, 1H) 2.73-2.84 (m, 2H) 3.48-3.64 (m, 3H) 3.85-3.95 (m, 1H) 4.02-4.20 (m, 2H) 6.32 (s, 2H) 6.54 (br s, 3H) 7.43 (dd, 1H) 7.61 (td, 1H) 7.70-7.82 (m, 1H) 8.47 (d, 1H)

### Example 406

### (rac)-2′-[6-amino-5-(trifluoromethyl)pyridin-3-yl]-N-[2-(6-methylpyridin-3-yl)propan-2-yl]-5′,6′-dihydrospiro[pyrrolidine-3,4′-pyrrolo[1,2-b]pyrazole]-1-carboxamide

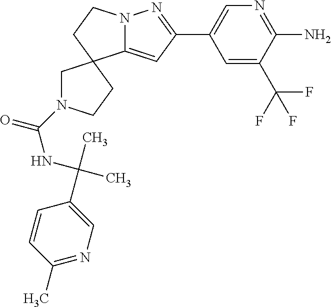 [SMILES: Cc1ccc(C(C)(C)NC(=O)N2CCC3(CCn4nc(-c5cnc(N)c(C(F)(F)F)c5)cc43)C2)cn1]

Example 406 was prepared in analogy to Example 404 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (112 mg, 284 μmol) and 5-(2-isocyanatopropan-2-yl)-2-methylpyridine (60.0 mg, 340 μmol).

LC-MS (Method 1): Rt=1.00 min; MS (ESIpos): m/z=500 [M+H]⁺

¹H NMR (400 MHz, DMSO-d₆) δ ppm 1.56 (d, 6H) 2.00-2.13 (m, 2H) 2.40 (s, 3H) 2.53 (br s, 2H) 3.41-3.50 (m, 3H) 3.55 (br s, 1H) 4.19 (t, 2H) 6.13 (s, 1H) 6.45 (s, 1H) 6.55 (s, 2H) 7.10 (d, 1H) 7.59 (dd, 1H) 8.02 (d, 1H) 8.43 (d, 1H) 8.60 (d, 1H)

### Example 407

### (rac)-2′-[6-amino-5-(trifluoromethyl)pyridin-3-yl]-N-(1-phenylcyclohexyl)-5′,6′-dihydrospiro[pyrrolidine-3,4′-pyrrolo[1,2-b]pyrazole]-1-carboxamide

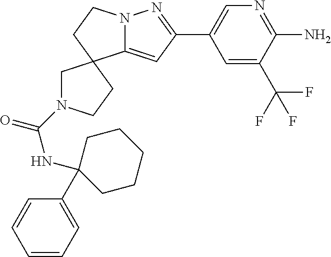 [SMILES: Nc1ncc(-c2cc3n(n2)CCC32CCN(C(=O)NC3(c4ccccc4)CCCCC3)C2)cc1C(F)(F)F]

Example 407 was prepared in analogy to Example 404 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (98.4 mg, 248 μmol) and (1-isocyanatocyclohexyl)benzene (60.0 mg, 298 μmol).

LC-MS (Method 1): Rt=1.27 min; MS (ESIpos): m/z=525 [M+H]⁺

¹H NMR (400 MHz, DMSO-d₆) δ ppm 1.23 (br s, 1H) 1.47-1.70 (m, 7H) 2.02-2.14 (m, 2H) 2.37 (br s, 2H) 2.52-2.60 (m, 2H) 3.43-3.55 (m, 3H) 3.56-3.65 (m, 1H) 4.20 (t, 2H) 5.78 (s, 1H) 6.42 (s, 1H) 6.56 (s, 2H) 7.10-7.16 (m, 1H) 7.25 (t, 2H) 7.38 (d, 2H) 7.99 (d, 1H) 8.58 (d, 1H)

### Example 408

### (rac)-2′-[6-amino-5-(trifluoromethyl)pyridin-3-yl]-N-[2-(5-fluoropyridin-2-yl)propan-2-yl]-5′,6′-dihydrospiro[pyrrolidine-3,4′-pyrrolo[1,2-b]pyrazole]-1-carboxamide

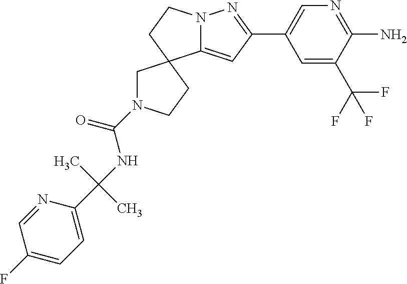 [SMILES: CC(C)(NC(=O)N1CCC2(CCn3nc(-c4cnc(N)c(C(F)(F)F)c4)cc32)C1)c1ccc(F)cn1]

Example 408 was prepared in analogy to Example 404 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (110 mg, 277 μmol) and 5-fluoro-2-(2-isocyanatopropan-2-yl)pyridine (60.0 mg, 333 μmol).

LC-MS (Method 1): Rt=1.06 min; MS (ESIpos): m/z=504 [M+H]⁺

¹H NMR (400 MHz, DMSO-d₆) δ ppm 1.57 (s, 6H) 2.01-2.14 (m, 2H) 2.53-2.58 (m, 2H) 3.41-3.49 (m, 3H) 3.50-3.63 (m, 1H) 4.20 (t, 2H) 6.30 (s, 1H) 6.49 (s, 1H) 6.55 (s, 2H) 7.49 (dd, 1H) 7.60 (td, 1H) 8.02 (d, 1H) 8.42 (d, 1H) 8.60 (d, 1H)

### Example 409

### 2′-[6-amino-5-(trifluoromethyl)pyridin-3-yl]-N-[(1R)-2,2,2-trifluoro-1-methoxy-1-phenylethyl]-5′,6′-dihydrospiro[pyrrolidine-3,4′-pyrrolo[1,2-b]pyrazole]-1-carboxamide (Mixture of Stereoisomers)

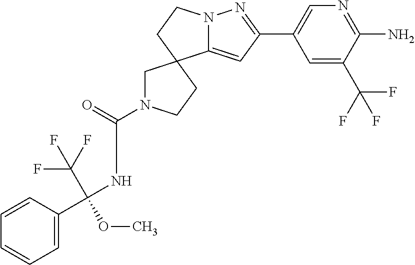 [SMILES: CO[C@](NC(=O)N1CCC2(CCn3nc(-c4cnc(N)c(C(F)(F)F)c4)cc32)C1)(c1ccccc1)C(F)(F)F]

Example 409 was prepared in analogy to Example 404 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (85.7 mg, 216 μmol) and [(1S)-2,2,2-trifluoro-1-isocyanato-1-methoxyethyl]benzene (60.0 mg, 260 μmol).

LC-MS (Method 1): Rt=1.18 min; MS (ESIpos): m/z=555 [M+H]⁺

¹H NMR (400 MHz, DMSO-d₆) δ ppm 1.21-1.30 (m, 1H) 2.09 (br s, 2H) 2.53-2.61 (m, 2H) 3.41-3.47 (m, 3H) 3.49 (br s, 1H) 3.57-3.76 (m, 2H) 4.22 (br t, 2H) 6.34-6.64 (m, 3H) 7.08 (s, 1H) 7.29-7.40 (m, 3H) 7.41-7.46 (m, 1H) 7.50 (br d, 1H) 8.01 (dd, 1H) 8.59 (dd, 1H)

### Example 410

### (rac)-2′-[6-amino-5-(trifluoromethyl)pyridin-3-yl]-N-[2-(pyrimidin-4-yl)propan-2-yl]-5′,6′-dihydrospiro[pyrrolidine-3,4′-pyrrolo[1,2-b]pyrazole]-1-carboxamide

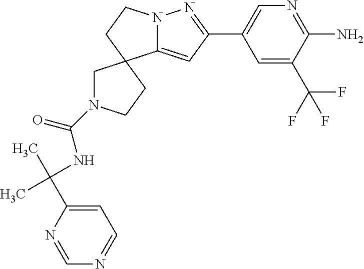 [SMILES: CC(C)(NC(=O)N1CCC2(CCn3nc(-c4cnc(N)c(C(F)(F)F)c4)cc32)C1)c1ccncn1]

Example 410 was prepared in analogy to Example 404 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (110 mg, 306 μmol) and 4-(2-isocyanatopropan-2-yl)pyrimidine (60.0 mg, 368 μmol).

LC-MS (Method 1): Rt=0.91 min; MS (ESIpos): m/z=487 [M+H]⁺

¹H NMR (400 MHz, DMSO-d₆) δ ppm 1.54 (s, 6H) 2.00-2.15 (m, 2H) 2.52-2.58 (m, 2H) 3.36-3.50 (m, 3H) 3.51-3.65 (m, 1H) 4.20 (t, 2H) 6.36 (s, 1H) 6.49 (s, 1H) 6.56 (s, 2H) 7.50 (dd, 1H) 8.02 (d, 1H) 8.60 (d, 1H) 8.64 (d, 1H) 9.05 (d, 1H)

### Example 411

### (rac)-2′-[6-amino-5-(trifluoromethyl)pyridin-3-yl]-N-[2-(pyrimidin-5-yl)propan-2-yl]-5′,6′-dihydrospiro[pyrrolidine-3,4′-pyrrolo[1,2-b]pyrazole]-1-carboxamide

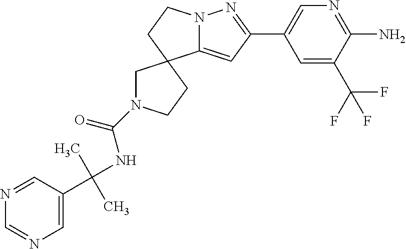 [SMILES: CC(C)(NC(=O)N1CCC2(CCn3nc(-c4cnc(N)c(C(F)(F)F)c4)cc32)C1)c1cncnc1]

Example 411 was prepared in analogy to Example 404 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (121 mg, 306 μmol) and 5-(2-isocyanatopropan-2-yl)pyrimidine (60.0 mg, 368 μmol).

LC-MS (Method 1): Rt=0.89 min; MS (ESIpos): m/z=487 [M+H]⁺

¹H NMR (400 MHz, DMSO-d₆) δ ppm 1.57-1.63 (m, 6H) 2.01-2.13 (m, 2H) 2.52-2.57 (m, 2H) 3.39-3.51 (m, 3H) 3.56 (br dd, 1H) 4.19 (t, 2H) 6.28 (s, 1H) 6.45 (s, 1H) 6.55 (s, 2H) 8.02 (d, 1H) 8.60 (d, 1H) 8.76 (s, 2H) 8.98 (s, 1H)

### Example 412

### (rac)-{2′-[6-amino-5-(trifluoromethyl)pyridin-3-yl]-5′,6′-dihydrospiro[pyrrolidine-3,4′-pyrrolo[1,2-b]pyrazol]-1-yl}(3-phenyloxetan-3-yl)methanone

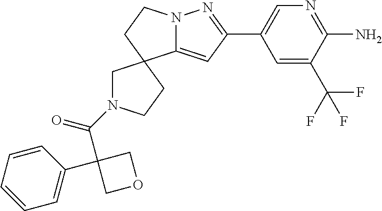 [SMILES: Nc1ncc(-c2cc3n(n2)CCC32CCN(C(=O)C3(c4ccccc4)COC3)C2)cc1C(F)(F)F]

Example 412 was prepared in analogy to Example 404 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (113 mg, 285 μmol) and 3-isocyanato-3-phenyloxetane (60.0 mg, 342 μmol).

LC-MS (Method 1): Rt=1.03 min; MS (ESIpos): m/z=484 [M+H]⁺

¹H NMR (400 MHz, DMSO-d₆) δ ppm 1.91-2.06 (m, 2H) 2.36-2.47 (m, 1H) 2.94-3.07 (m, 2H) 3.58-3.68 (m, 2H) 3.92-4.00 (m, 1H) 4.04-4.21 (m, 1H) 4.63-4.72 (m, 3H) 5.17 (dd, 1H) 6.50-6.60 (m, 2H) 7.23-7.30 (m, 1H) 7.35-7.41 (m, 2H) 7.46-7.53 (m, 3H) 7.55-7.60 (m, 1H) 7.93-7.99 (m, 1H) 8.52-8.57 (m, 1H)

### Example 413

### (rac)-1-{2′-[6-amino-5-(trifluoromethyl)pyridin-3-yl]-5′,6′-dihydrospiro[pyrrolidine-3,4′-pyrrolo[1,2-b]pyrazol]-1-yl}-2-(3,5-difluorophenyl)-2-methylpropan-1-one

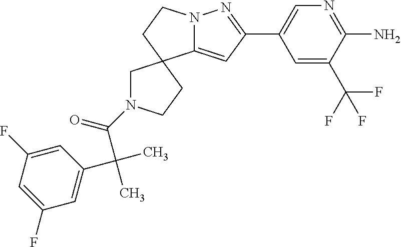 [SMILES: CC(C)(C(=O)N1CCC2(CCn3nc(-c4cnc(N)c(C(F)(F)F)c4)cc32)C1)c1cc(F)cc(F)c1]

Example 413 was prepared in analogy to Example 404 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (100 mg, 254 μmol) and 1,3-difluoro-5-(2-isocyanatopropan-2-yl)benzene (60.0 mg, 304 μmol).

LC-MS (Method 1): Rt=1.22 min; MS (ESIpos): m/z=506 [M+H]⁺

¹H NMR (400 MHz, DMSO-d₆) δ ppm 1.40 (s, 2H) 1.43-1.54 (m, 4H) 1.93 (br t, 2H) 2.15-2.27 (m, 1H) 2.36-2.44 (m, 1H) 2.84-2.99 (m, 1H) 3.06 (br d, 1H) 3.52-3.67 (m, 2H) 3.95 (dt, 1H) 4.02-4.23 (m, 2H) 6.49-6.62 (m, 2H) 6.91-7.09 (m, 3H) 7.96-8.04 (m, 1H) 8.57 (br s, 1H)

### Example 414

### (rac)-1-{2′-[6-amino-5-(trifluoromethyl)pyridin-3-yl]-5′,6′-dihydrospiro[pyrrolidine-3,4′-pyrrolo[1,2-b]pyrazol]-1-yl}-2-methyl-2-(6-methylpyridin-3-yl)propan-1-one

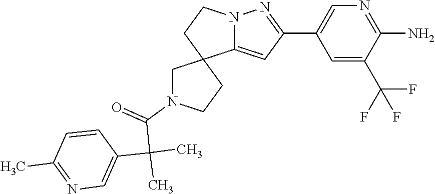 [SMILES: Cc1ccc(C(C)(C)C(=O)N2CCC3(CCn4nc(-c5cnc(N)c(C(F)(F)F)c5)cc43)C2)cn1]

Example 414 was prepared in analogy to Example 404 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (112 mg, 284 μmol) and 5-(2-isocyanatopropan-2-yl)-2-methylpyridine (60.0 mg, 340 μmol).

LC-MS (Method 1): Rt=1.01 min; MS (ESIpos): m/z=485 [M+H]⁺

¹H NMR (400 MHz, DMSO-d₆) δ ppm 1.40-1.54 (m, 6H) 1.81-2.02 (m, 2H) 2.05-2.15 (m, 1H) 2.29-2.44 (m, 3H) 2.52-2.55 (m, 1H) 2.83-3.02 (m, 2H) 3.51-3.65 (m, 2H) 3.81-3.90 (m, 1H) 4.01-4.20 (m, 1H) 6.26 (s, 1H) 6.49-6.61 (m, 2H) 7.10-7.30 (m, 1H) 7.50-7.65 (m, 1H) 7.99 (br s, 1H) 8.30-8.38 (m, 1H) 8.54-8.60 (m, 1H)

### Example 415

### (rac)-2′-[6-amino-5-(trifluoromethyl)pyridin-3-yl]-N-(3-phenyloxetan-3-yl)-5′,6′-dihydrospiro[pyrrolidine-3,4′-pyrrolo[1,2-b]pyrazole]-1-carboxamide

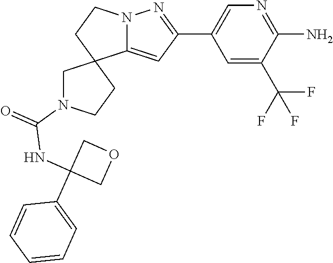 [SMILES: Nc1ncc(-c2cc3n(n2)CCC32CCN(C(=O)NC3(c4ccccc4)COC3)C2)cc1C(F)(F)F]

Example 415 was prepared in analogy to Example 404 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (113 mg, 285 μmol) and 3-isocyanato-3-phenyloxetane (60.0 mg, 342 μmol).

LC-MS (Method 1): Rt=0.97 min; MS (ESIpos): m/z=499 [M+H]⁺

¹H NMR (400 MHz, DMSO-d₆) δ ppm 2.11 (br d, 2H) 2.57 (t, 2H) 3.48 (s, 3H) 3.52-3.67 (m, 1H) 4.20 (t, 2H) 4.65 (dd, 2H) 4.90 (dd, 2H) 6.48 (s, 1H) 6.56 (s, 2H) 7.22-7.32 (m, 2H) 7.36 (t, 2H) 7.53 (d, 2H) 8.02 (d, 1H) 8.60 (d, 1H)

### Example 416

### (rac)-2′-[6-amino-5-(trifluoromethyl)pyridin-3-yl]-N-(propan-2-yl)-5′,6′-dihydrospiro[pyrrolidine-3,4′-pyrrolo[1,2-b]pyrazole]-1-carboxamide

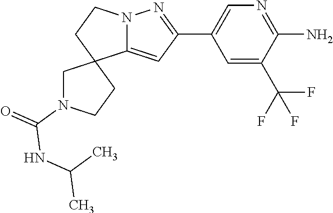 [SMILES: CC(C)NC(=O)N1CCC2(CCn3nc(-c4cnc(N)c(C(F)(F)F)c4)cc32)C1]

(rac)-5-[5′,6′-dihydrospiro[pyrrolidine-3,4′-pyrrolo[1,2-b]pyrazol]-2′-yl]-3-(trifluoromethyl)pyridin-2-amine hydrochloride (3.30 g, 9.17 mmol) was solubilised in dichloromethane (75 mL), N,N-diisopropylethylamine (4.8 mL, 28 mmol) and 2-isocyanatopropane (390 mg, 4.59 mmol) were added and the mixture was stirred for 1 h at rt under argon. The mixture was evaporated and the residue was purified by flash chromatography. The resulting crude was diluted with brine and water and extracted 3× with DCM. The organic layer was dried over a silicone filter and concentrated under reduced pressure to give 2.10 g (99% purity, 55% yield) of the title compound.

LC-MS (Method 1): Rt=0.96 min; MS (ESIpos): m/z=409 [M+H]⁺

¹H NMR (400 MHz, DMSO-d₆) δ ppm 1.06 (dd, 6H) 1.99-2.10 (m, 2H) 2.52-2.55 (m, 2H) 3.36-3.43 (m, 3H) 3.45-3.55 (m, 1H) 3.71-3.80 (m, 1H) 4.18 (t, 2H) 5.82 (d, 1H) 6.47 (s, 1H) 6.54 (s, 2H) 8.01 (d, 1H) 8.59 (d, 1H)

### Example 417

### (rac)-2′-[6-amino-5-(trifluoromethyl)pyridin-3-yl]-N-cyclobutyl-5′,6′-dihydrospiro[pyrrolidine-3,4′-pyrrolo[1,2-b]pyrazole]-1-carboxamide

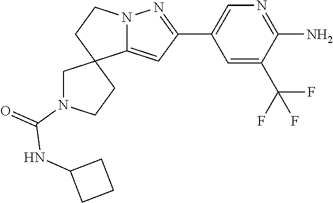 [SMILES: Nc1ncc(-c2cc3n(n2)CCC32CCN(C(=O)NC3CCC3)C2)cc1C(F)(F)F]

Example 417 was prepared in analogy to

Example 416 using (rac)-5-[5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and isocyanatocyclobutane (20.2 mg, 208 μmol).

LC-MS (Method 1): Rt=0.98 min; MS (ESIpos): m/z=421 [M+H]⁺

¹H NMR (400 MHz, DMSO-d₆) δ ppm 1.45-1.62 (m, 2H) 1.85-1.99 (m, 2H) 2.00-2.07 (m, 2H) 3.07 (br d, 2H) 3.40-3.61 (m, 7H) 4.17 (t, 2H) 6.34 (d, 1H) 6.45-6.50 (m, 1H) 6.53 (s, 2H) 8.02 (d, 1H) 8.59 (d, 1H)

### Example 418

### (3R)-2′-[6-amino-5-(trifluoromethyl)pyridin-3-yl]-N-ethyl-5′,6′-dihydrospiro[pyrrolidine-3,4′-pyrrolo[1,2-b]pyrazole]-1-carboxamide

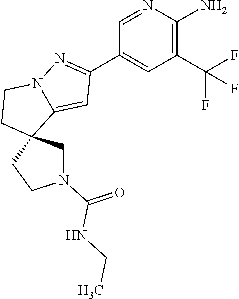 [SMILES: CCNC(=O)N1CC[C@@]2(CCn3nc(-c4cnc(N)c(C(F)(F)F)c4)cc32)C1]

Example 418 was prepared in analogy to

Example 416 using 5-[(3R)-5′,6′-dihydrospiro[pyrrolidine-3,4′-pyrrolo[1,2-b]pyrazol]-2′-yl]-3-(trifluoromethyl)pyridin-2-amine hydrochloride (54.0 mg, 150 μmol, stereoisomer 2) and isocyanatoethane (12 μL, 150 μmol)

¹H-NMR (400 MHz, DMSO-d₆): δ [ppm]=1.02 (t, 3H), 2.00-2.10 (m, 2H), 2.52-2.55 (m, 2H), 3.01-3.10 (m, 2H), 3.36-3.43 (m, 3H), 3.46-3.53 (m, 1H), 4.18 (t, 2H), 6.15 (t, 1H), 6.48 (s, 1H), 6.54 (s, 2H), 8.02 (d, 1H), 8.59 (d, 1H).

[α]²⁰_{D}: +51.8° (c=1.00, DMSO)

LC-MS (method 1): Rₜ=0.89 min; MS (ESIpos): m/z=395 [M+H]⁺

### Example 419

### (3S)-2′-[6-amino-5-(trifluoromethyl)pyridin-3-yl]-N-ethyl-5′,6′-dihydrospiro[pyrrolidine-3,4′-pyrrolo[1,2-b]pyrazole]-1-carboxamide

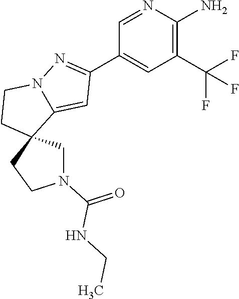 [SMILES: CCNC(=O)N1CC[C@]2(CCn3nc(-c4cnc(N)c(C(F)(F)F)c4)cc32)C1]

Example 419 was prepared in analogy to

Example 416 using 5-[(3S)-5′,6′-dihydrospiro[pyrrolidine-3,4′-pyrrolo[1,2-b]pyrazol]-2′-yl]-3-(trifluoromethyl)pyridin-2-amine hydrochloride (49.0 mg, 136 μmol) and isocyanatoethane (11 μL, 136 μmol).

¹H-NMR (400 MHz, DMSO-d₆): δ [ppm]=1.02 (t, 3H), 2.01-2.10 (m, 2H), 2.52-2.55 (m, 2H), 3.01-3.09 (m, 2H), 3.36-3.43 (m, 3H), 3.46-3.55 (m, 1H), 4.18 (t, 2H), 6.15 (t, 1H), 6.48 (s, 1H), 6.54 (s, 2H), 8.02 (d, 1H), 8.59 (d, 1H).

[α]²⁰_{D}: −46.6° (c=1.00, DMSO)

LC-MS (method 1): Rₜ=0.89 min; MS (ESIpos): m/z=396 [M+H]⁺

### Example 420

### (3R)-2′-[6-amino-5-(trifluoromethyl) pyridin-3-yl]-N-(propan-2-yl)-5′,6′-dihydrospiro[pyrrolidine-3,4′-pyrrolo[1,2-b]pyrazole]-1-carboxamide

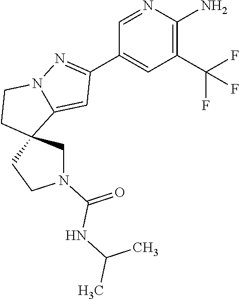 [SMILES: CC(C)NC(=O)N1CC[C@@]2(CCn3nc(-c4cnc(N)c(C(F)(F)F)c4)cc32)C1]

Example 420 was prepared in analogy to

Example 416 using 5-[(3R)-5′,6′-dihydrospiro[pyrrolidine-3,4′-pyrrolo[1,2-b]pyrazol]-2′-yl]-3-(trifluoromethyl)pyridin-2-amine hydrochloride (68.0 mg, 189 μmol) and 2-isocyanatopropane (13 mg, 0.19 mmol).

LC-MS (method 1): Rₜ=0.94 min; MS (ESIpos): m/z=409 [M+H]⁺

¹H-NMR (400 MHz, DMSO-d₆): δ [ppm]=1.06 (dd, 6H), 1.99-2.10 (m, 2H), 2.52-2.55 (m, 2H), 3.37-3.44 (m, 3H), 3.45-3.56 (m, 1H), 3.71-3.80 (m, 1H), 4.18 (t, 2H), 5.82 (d, 1H), 6.47 (s, 1H), 6.54 (s, 2H), 8.02 (d, 1H), 8.59 (d, 1H).

[α]²⁰_{D}: +47.3° (c=1.00, DMSO)

### Example 421

### (3S)-2′-[6-amino-5-(trifluoromethyl)pyridin-3-yl]-N-(propan-2-yl)-5′,6′-dihydrospiro[pyrrolidine-3,4′-pyrrolo[1,2-b]pyrazole]-1-carboxamide

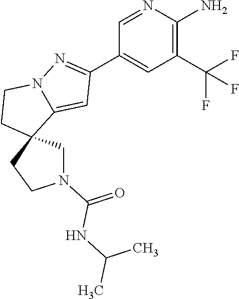 [SMILES: CC(C)NC(=O)N1CC[C@]2(CCn3nc(-c4cnc(N)c(C(F)(F)F)c4)cc32)C1]

Example 421 was prepared in analogy to

Example 416 using 5-[(3S)-5′,6′-dihydrospiro[pyrrolidine-3,4′-pyrrolo[1,2-b]pyrazol]-2′-yl]-3-(trifluoromethyl)pyridin-2-amine hydrochloride (77.0 mg, 214 μmol, stereoisomer 1) and 2-isocyanatopropane (15 mg, 0.21 mmol).

[α]²⁰_{D}: −46.4° (c=1.00, DMSO)

¹H-NMR (400 MHz, DMSO-d₆): δ [ppm]=1.06 (dd, 6H), 2.00-2.10 (m, 2H), 2.52-2.56 (m, 2H), 3.36-3.43 (m, 3H), 3.51 (dt, 1H), 3.71-3.80 (m, 1H), 4.18 (t, 2H), 5.82 (d, 1H), 6.47 (s, 1H), 6.54 (s, 2H), 8.02 (d, 1H), 8.59 (d, 1H).

LC-MS (method 1): Rₜ=0.94 min; MS (ESIpos): m/z=409 [M+H]⁺

### Example 422

### (rac)-2-[6-amino-5-(trifluoromethyl)pyridin-3-yl]-N-cyclobutyl-6,7-dihydrospiro[pyrazolo[5,1-c][1,4]oxazine-4,3′-pyrrolidine]-1′-carboxamide

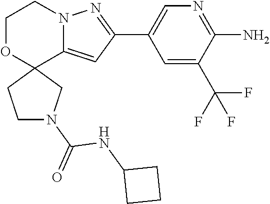 [SMILES: Nc1ncc(-c2cc3n(n2)CCOC32CCN(C(=O)NC3CCC3)C2)cc1C(F)(F)F]

Example 422 was prepared in analogy to

Example 416 using (rac)-5-[6,7-dihydrospiro[pyrazolo[5,1-c][1,4]oxazine-4,3′-pyrrolidin]-2-yl]-3-(trifluoromethyl)pyridin-2-amine (75.0 mg, 221 μmol) and isocyanatocyclobutane (21.5 mg, 221 μmol).

LC-MS (Method 1): Rt=0.95 min; MS (ESIpos): m/z=437 [M+H]⁺

¹H NMR (400 MHz, DMSO-d₆) δ ppm 1.46-1.64 (m, 2H) 1.85-2.02 (m, 2H) 2.06-2.14 (m, 2H) 2.16-2.28 (m, 1H) 2.29-2.39 (m, 1H) 3.35-3.49 (m, 2H) 3.55 (t, 1H) 3.73-3.81 (m, 1H) 4.02-4.18 (m, 5H) 6.32 (d, 1H) 6.60 (s, 2H) 6.74 (s, 1H) 8.01 (d, 1H) 8.58 (d, 1H)

### Example 423

### (rac)-2-[6-amino-5-(trifluoromethyl)pyridin-3-yl]-N-ethyl-6,7-dihydrospiro[pyrazolo[5,1-c][1,4]oxazine-4,3′-pyrrolidine]-1′-carboxamide

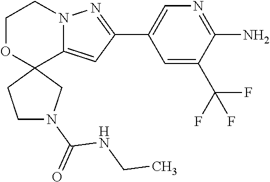 [SMILES: CCNC(=O)N1CCC2(C1)OCCn1nc(-c3cnc(N)c(C(F)(F)F)c3)cc12]

Example 423 was prepared in analogy to

Example 416 using (rac)-5-[6,7-dihydrospiro[pyrazolo[5,1-c][1,4]oxazine-4,3′-pyrrolidin]-2-yl]-3-(trifluoromethyl)pyridin-2-amine (75.0 mg, 221 μmol) and isocyanatoethane (17 μL, 220 μmol).

LC-MS (Method 1): Rt=0.87 min; MS (ESIpos): m/z=411 [M+H]⁺

¹H NMR (400 MHz, DMSO-d₆) δ ppm 1.02 (t, 3H) 2.22 (br d, 1H) 2.30-2.35 (m, 1H) 3.05 (dd, 2H) 3.10-3.17 (m, 1H) 3.35-3.46 (m, 2H) 3.76 (br d, 1H) 4.03-4.17 (m, 4H) 6.18 (t, 1H) 6.60 (s, 2H) 6.74 (s, 1H) 8.01 (d, 1H) 8.58 (d, 1H)

### Example 424

### (rac)-2-[6-amino-5-(trifluoromethyl)pyridin-3-yl]-N-(propan-2-yl)-6,7-dihydrospiro[pyrazolo[5,1-c][1,4]oxazine-4,3′-pyrrolidine]-1′-carboxamide

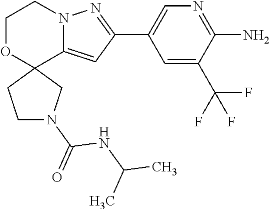 [SMILES: CC(C)NC(=O)N1CCC2(C1)OCCn1nc(-c3cnc(N)c(C(F)(F)F)c3)cc12]

Example 424 was prepared in analogy to

Example 416 using (rac)-5-[6,7-dihydrospiro[pyrazolo[5,1-c][1,4]oxazine-4,3′-pyrrolidin]-2-yl]-3-(trifluoromethyl)pyridin-2-amine (75.0 mg, 221 μmol) and 2-isocyanatopropane (18.8 mg, 221 μmol).

LC-MS (Method 1): Rt=0.93 min; MS (ESIpos): m/z=425 [M+H]⁺

¹H NMR (400 MHz, DMSO-d₆) δ ppm 1.06 (d, 6H) 1.26 (br s, 4H) 2.17-2.26 (m, 1H) 2.29-2.38 (m, 1H) 3.43 (d, 1H) 3.70-3.81 (m, 2H) 4.11-4.17 (m, 2H) 5.84 (d, 1H) 6.60 (s, 2H) 6.75 (s, 1H) 8.01 (d, 1H) 8.58 (d, 1H)

### Example 425

### 2′-[6-amino-5-(trifluoromethyl)pyridin-3-yl]-N-cyclobutyl-5′,6′-dihydrospiro[azetidine-3,4′-pyrrolo[1,2-b]pyrazole]-1-carboxamide

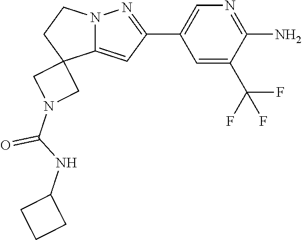 [SMILES: Nc1ncc(-c2cc3n(n2)CCC32CN(C(=O)NC3CCC3)C2)cc1C(F)(F)F]

Example 425 was prepared in analogy to

Example 416 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and isocyanatocyclobutane (23.5 mg, 242 μmol).

LC-MS (Method 1): Rt=0.93 min; MS (ESIpos): m/z=407 [M+H]⁺

¹H NMR (400 MHz, DMSO-d₆) δ ppm 1.46-1.63 (m, 2H) 1.83-1.98 (m, 2H) 2.04-2.17 (m, 2H) 2.83 (t, 2H) 3.97 (s, 4H) 4.04-4.16 (m, 3H) 6.55 (s, 2H) 6.67 (d, 1H) 6.72 (s, 1H) 8.04 (d, 1H) 8.61 (d, 1H)

### Example 426

### 2′-[6-amino-5-(trifluoromethyl)pyridin-3-yl]-N-ethyl-5′,6′-dihydrospiro[azetidine-3,4′-pyrrolo[1,2-b]pyrazole]-1-carboxamide

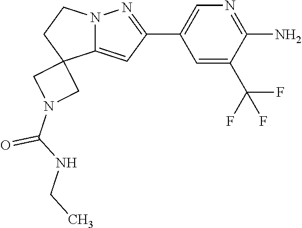 [SMILES: CCNC(=O)N1CC2(CCn3nc(-c4cnc(N)c(C(F)(F)F)c4)cc32)C1]

Example 426 was prepared in analogy to

Example 416 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and isocyanatoethane (19 μL, 240 μmol).

LC-MS (Method 1): Rt=0.83 min; MS (ESIpos): m/z=381 [M+H]⁺

¹H NMR (400 MHz, DMSO-d₆) δ ppm 1.01 (t, 3H) 2.84 (t, 2H) 2.98-3.08 (m, 2H) 3.97 (s, 4H) 4.12 (t, 2H) 6.47 (t, 1H) 6.55 (br s, 2H) 6.72 (s, 1H) 8.04 (d, 1H) 8.62 (d, 1H)

### Example 427

### 2′-[6-amino-5-(trifluoromethyl)pyridin-3-yl]-N-(propan-2-yl)-5′,6′-dihydrospiro[azetidine-3,4′-pyrrolo[1,2-b]pyrazole]-1-carboxamide

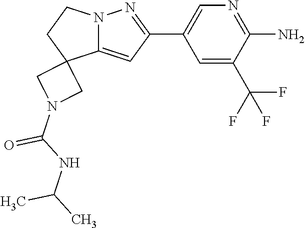 [SMILES: CC(C)NC(=O)N1CC2(CCn3nc(-c4cnc(N)c(C(F)(F)F)c4)cc32)C1]

Example 427 was prepared in analogy to

Example 416 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 2-isocyanatopropane (20.6 mg, 242 μmol).

LC-MS (Method 1): Rt=0.92 min; MS (ESIpos): m/z=395 [M+H]⁺

¹H NMR (400 MHz, DMSO-d₆) δ ppm 1.06 (d, 6H) 2.83 (t, 2H) 3.67-3.78 (m, 1H) 3.97 (s, 4H) 4.12 (t, 2H) 6.23 (d, 1H) 6.49-6.60 (m, 2H) 6.73 (s, 1H) 8.00-8.08 (m, 1H) 8.59-8.65 (m, 1H)

### Example 428

### (rac)-2′-(6-amino-5-cyanopyridin-3-yl)-N-[2-(3-chloropyridin-4-yl)propan-2-yl]-5′,6′-dihydrospiro[pyrrolidine-3,4′-pyrrolo[1,2-b]pyrazole]-1-carboxamide

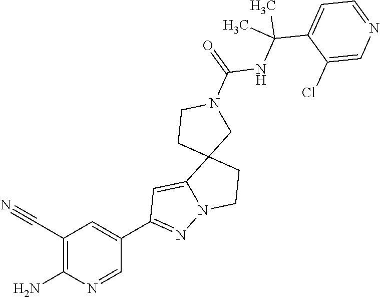 [SMILES: CC(C)(NC(=O)N1CCC2(CCn3nc(-c4cnc(N)c(C#N)c4)cc32)C1)c1ccncc1Cl]

Example 428 was prepared in analogy to Example 404 using (rac)-2-amino-5-[5′,6′-dihydrospiro[pyrrolidine-3,4′-pyrrolo[1,2-b]pyrazol]-2′-yl]pyridine-3-carbonitrile hydrochloride (107 mg, 339 μmol) and 3-chloro-4-(2-isocyanatopropan-2-yl)pyridine (80.0 mg, 407 μmol).

¹H NMR (400 MHz, DMSO-d₆) δ ppm 1.65 (s, 6H) 1.97-2.14 (m, 2H) 2.52-2.57 (m, 2H) 3.35-3.54 (m, 4H) 4.18 (t, 2H) 6.33 (s, 1H) 6.43 (s, 1H) 7.00 (s, 2H) 7.43 (d, 1H) 8.13 (d, 1H) 8.40 (d, 1H) 8.42 (s, 1H) 8.60 (d, 1H)

LC-MS (Method 1): Rₜ=0.90 min; MS (ESIpos): m/z=477 [M+H]⁺

### Example 429

### (rac)-2′-{6-amino-5-[(2-chlorophenyl)methoxy]pyridin-3-yl}-N-ethyl-5′,6′-dihydrospiro[pyrrolidine-3,4′-pyrrolo[1,2-b]pyrazole]-1-carboxamide

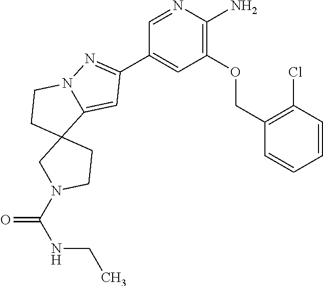 [SMILES: CCNC(=O)N1CCC2(CCn3nc(-c4cnc(N)c(OCc5ccccc5Cl)c4)cc32)C1]

Example 429 was prepared in analogy to

Example 416 using (rac)-3-[(2-chlorophenyl)methoxy]-5-[5′,6′-dihydrospiro[pyrrolidine-3,4′-pyrrolo[1,2-b]pyrazol]-2′-yl]pyridin-2-amine-hydrochloride salt (70.0 mg, 149 μmol) and isocyanatoethane (13 μL, 160 μmol).

LC-MS (Method 1): Rₜ=1.02 min; MS (ESIpos): m/z=467 [M+H]⁺

¹H NMR (400 MHz, DMSO-d₆) δ ppm 0.98-1.06 (m, 3H) 1.98-2.12 (m, 2H) 2.52-2.55 (m, 1H) 3.01-3.09 (m, 2H) 3.35-3.44 (m, 3H) 3.45-3.54 (m, 1H) 4.10-4.20 (m, 2H) 5.22 (s, 2H) 5.82 (s, 2H) 6.15 (t, 1H) 6.36 (s, 1H) 7.37-7.42 (m, 2H) 7.45 (d, 1H) 7.49-7.56 (m, 1H) 7.66-7.76 (m, 1H) 7.91-7.98 (m, 1H)

### Example 430

### (rac)-2′-{6-amino-5-[(2-chlorophenyl)methoxy]pyridin-3-yl}-N-(propan-2-yl)-5′,6′-dihydrospiro[pyrrolidine-3,4′-pyrrolo[1,2-b]pyrazole]-1-carboxamide

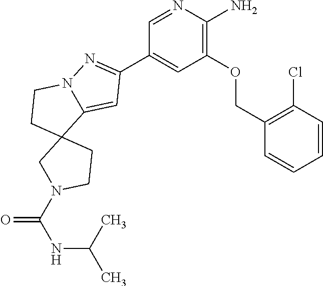 [SMILES: CC(C)NC(=O)N1CCC2(CCn3nc(-c4cnc(N)c(OCc5ccccc5Cl)c4)cc32)C1]

Example 430 was prepared in analogy to

Example 416 using (rac)-3-[(2-chlorophenyl)methoxy]-5-[5′,6′-dihydrospiro[pyrrolidine-3,4′-pyrrolo[1,2-b]pyrazol]-2′-yl]pyridin-2-amine-hydrochloride salt (68.0 mg, 145 μmol) and 2-isocyanatopropane (13.0 mg, 152 μmol).

LC-MS (Method 1): Rt=1.07 min; MS (ESIpos): m/z=481 [M+H]⁺

¹H NMR (400 MHz, DMSO-d₆) δ ppm 1.02-1.11 (m, 6H) 1.23 (s, 1H) 1.97-2.11 (m, 2H) 2.52-2.55 (m, 1H) 3.36-3.43 (m, 3H) 3.45-3.55 (m, 1H) 3.70-3.80 (m, 1H) 4.11-4.20 (m, 2H) 5.22 (s, 2H) 5.78-5.87 (m, 3H) 6.36 (s, 1H) 7.34-7.46 (m, 3H) 7.49-7.55 (m, 1H) 7.66-7.76 (m, 1H) 7.91-7.98 (m, 1H)

### Example 431

### (rac)-2′-{6-amino-5-[(2-chlorophenyl)methoxy]pyridin-3-yl}-N-cyclobutyl-5′,6′-dihydrospiro[pyrrolidine-3,4′-pyrrolo[1,2-b]pyrazole]-1-carboxamide

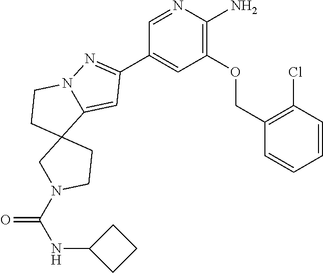 [SMILES: Nc1ncc(-c2cc3n(n2)CCC32CCN(C(=O)NC3CCC3)C2)cc1OCc1ccccc1Cl]

Example 431 was prepared in analogy to

Example 416 using (rac)-3-[(2-chlorophenyl)methoxy]-5-[5′,6′-dihydrospiro[pyrrolidine-3,4′-pyrrolo[1,2-b]pyrazol]-2′-yl]pyridin-2-amine-hydrochloride salt (66.0 mg, 141 μmol) and isocyanatocyclobutane (14.4 mg, 148 μmol).

LC-MS (Method 1): Rt=1.09 min; MS (ESIpos): m/z=493 [M+H]⁺

¹H NMR (400 MHz, DMSO-d₆) δ ppm 1.46-1.63 (m, 2H) 1.86-1.93 (m, 1H) 1.93-1.99 (m, 1H) 2.00-2.04 (m, 1H) 2.06 (br d, 1H) 2.08-2.10 (m, 1H) 2.10-2.15 (m, 1H) 2.52-2.54 (m, 1H) 3.36-3.39 (m, 1H) 3.39-3.41 (m, 2H) 3.42 (br s, 1H) 3.46-3.54 (m, 1H) 4.06-4.19 (m, 3H) 5.22 (s, 2H) 5.82 (s, 2H) 6.27-6.37 (m, 2H) 7.35-7.54 (m, 4H) 7.66-7.76 (m, 1H) 7.91-7.98 (m, 1H)

### Example 432

### (rac)-2′-{6-amino-5-[(2-fluorophenyl)methoxy]pyridin-3-yl}-N-ethyl-5′,6′-dihydrospiro[pyrrolidine-3,4′-pyrrolo[1,2-b]pyrazole]-1-carboxamide

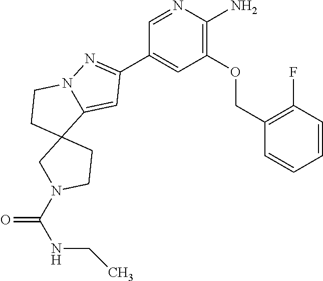 [SMILES: CCNC(=O)N1CCC2(CCn3nc(-c4cnc(N)c(OCc5ccccc5F)c4)cc32)C1]

Example 432 was prepared in analogy to

Example 416 using (rac)-5-[5′,6′-dihydrospiro[pyrrolidine-3,4′-pyrrolo[1,2-b]pyrazol]-2′-yl]-3-[(2-fluorophenyl)methoxy]pyridin-2-amine-hydrochloride salt (104 mg, 230 μmol) and isocyanatoethane (20 μL, 240 μmol).

LC-MS (Method 1): Rt=0.97 min; MS (ESIpos): m/z=451 [M+H]⁺

¹H NMR (400 MHz, DMSO-d₆) δ ppm 1.02 (t, 4H) 1.10-1.26 (m, 1H) 1.99-2.11 (m, 2H) 2.96-3.09 (m, 2H) 3.36-3.53 (m, 4H) 4.16 (t, 2H) 5.21 (s, 2H) 5.77 (s, 2H) 6.16 (t, 1H) 6.36 (s, 1H) 7.22-7.29 (m, 2H) 7.39-7.49 (m, 2H) 7.67 (td, 1H) 7.96 (d, 1H)

### Example 433

### (rac)-2′-{6-amino-5-[(2-fluorophenyl)methoxy]pyridin-3-yl}-N-(propan-2-yl)-5′,6′-dihydrospiro[pyrrolidine-3,4′-pyrrolo[1,2-b]pyrazole]-1-carboxamide

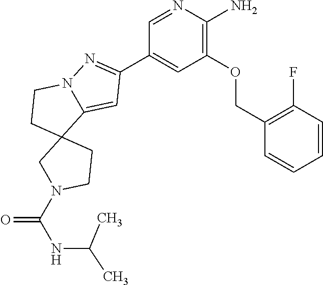 [SMILES: CC(C)NC(=O)N1CCC2(CCn3nc(-c4cnc(N)c(OCc5ccccc5F)c4)cc32)C1]

Example 433 was prepared in analogy to

Example 416 using (rac)-5-[5′,6′-dihydrospiro[pyrrolidine-3,4′-pyrrolo[1,2-b]pyrazol]-2′-yl]-3-[(2-fluorophenyl)methoxy]pyridin-2-amine-hydrochloride salt (104 mg, 230 μmol) and 2-isocyanatopropane (20.5 mg, 241 μmol).

LC-MS (Method 1): Rt=1.03 min; MS (ESIpos): m/z=465 [M+H]⁺

¹H NMR (400 MHz, DMSO-d₆) δ ppm 1.02-1.09 (m, 6H) 1.99-2.10 (m, 2H) 2.52-2.55 (m, 1H) 3.36-3.43 (m, 3H) 3.45-3.58 (m, 1H) 3.71-3.81 (m, 1H) 4.17 (t, 2H) 5.21 (s, 2H) 5.73-5.84 (m, 3H) 6.36 (s, 1H) 7.23-7.29 (m, 2H) 7.39-7.49 (m, 2H) 7.67 (td, 1H) 7.96 (d, 1H)

### Example 434

### (rac)-2′-{6-amino-5-[(2-fluorophenyl)methoxy]pyridin-3-yl}-N-cyclobutyl-5′,6′-dihydrospiro[pyrrolidine-3,4′-pyrrolo[1,2-b]pyrazole]-1-carboxamide

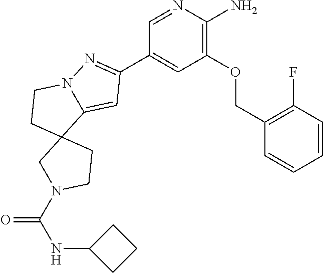 [SMILES: Nc1ncc(-c2cc3n(n2)CCC32CCN(C(=O)NC3CCC3)C2)cc1OCc1ccccc1F]

Example 434 was prepared in analogy to

Example 416 using (rac)-5-[5′,6′-dihydrospiro[pyrrolidine-3,4′-pyrrolo[1,2-b]pyrazol]-2′-yl]-3-[(2-fluorophenyl)methoxy]pyridin-2-amine-hydrochloride salt (104 mg, 230 μmol) and isocyanatocyclobutane (23.4 mg, 241 μmol).

LC-MS (Method 1): Rt=1.05 min; MS (ESIpos): m/z=477 [M+H]⁺

¹H NMR (400 MHz, DMSO-d₆) δ ppm 1.46-1.61 (m, 2H) 1.87-2.13 (m, 6H) 2.52-2.55 (m, 1H) 3.35-3.53 (m, 4H) 4.07-4.20 (m, 3H) 5.21 (s, 2H) 5.76 (s, 2H) 6.31 (d, 1H) 6.36 (s, 1H) 7.22-7.29 (m, 2H) 7.39-7.49 (m, 2H) 7.67 (td, 1H) 7.96 (d, 1H)

### Example 435

### (rac)-2′-{6-amino-5-[(2-fluorophenyl)methoxy]pyridin-3-yl}-N-benzyl-5′,6′-dihydrospiro[pyrrolidine-3,4′-pyrrolo[1,2-b]pyrazole]-1-carboxamide

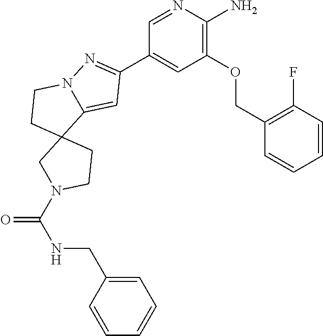 [SMILES: Nc1ncc(-c2cc3n(n2)CCC32CCN(C(=O)NCc3ccccc3)C2)cc1OCc1ccccc1F]

Example 435 was prepared in analogy to

Example 416 using (rac)-5-[5′,6′-dihydrospiro[pyrrolidine-3,4′-pyrrolo[1,2-b]pyrazol]-2′-yl]-3-[(2-fluorophenyl)methoxy]pyridin-2-amine-hydrochloride salt (104 mg, 230 μmol) and (isocyanatomethyl)benzene (32.1 mg, 241 μmol).

LC-MS (Method 1): Rt=1.09 min; MS (ESIpos): m/z=513 [M+H]⁺

¹H NMR (400 MHz, DMSO-d₆) δ ppm 2.04-2.14 (m, 2H) 3.46 (s, 3H) 3.53-3.61 (m, 1H) 4.17 (t, 2H) 4.26 (d, 3H) 5.21 (s, 2H) 5.79 (br s, 2H) 6.36 (s, 1H) 6.80 (t, 1H) 7.18-7.32 (m, 8H) 7.39-7.45 (m, 1H) 7.49 (s, 1H) 7.67 (t, 1H) 7.96 (d, 1H)

### Example 436

### (rac)-2′-{6-amino-5-[(3-fluorophenyl)methoxy]pyridin-3-yl}-N-ethyl-5′,6′-dihydrospiro[pyrrolidine-3,4′-pyrrolo[1,2-b]pyrazole]-1-carboxamide

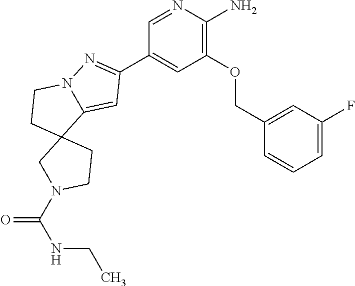 [SMILES: CCNC(=O)N1CCC2(CCn3nc(-c4cnc(N)c(OCc5cccc(F)c5)c4)cc32)C1]

Example 436 was prepared in analogy to

Example 416 using (rac)-5-[5′,6′-dihydrospiro[pyrrolidine-3,4′-pyrrolo[1,2-b]pyrazol]-2′-yl]-3-[(3-fluorophenyl)methoxy]pyridin-2-amine-hydrochloride salt (90.0 mg, 199 μmol) and isocyanatoethane (17 μL, 210 μmol).

LC-MS (Method 1): Rt=0.97 min; MS (ESIpos): m/z=451 [M+H]⁺

¹H NMR (400 MHz, DMSO-d₆) δ ppm 1.02 (t, 3H) 1.08-1.35 (m, 1H) 1.99-2.11 (m, 2H) 3.01-3.09 (m, 2H) 3.35-3.52 (m, 4H) 4.16 (t, 2H) 5.20 (s, 2H) 5.87 (s, 2H) 6.15 (t, 1H) 6.34 (s, 1H) 7.15 (t, 1H) 7.35-7.47 (m, 4H) 7.95 (d, 1H)

### Example 437

### (rac)-2′-{6-amino-5-[(3-fluorophenyl)methoxy]pyridin-3-yl}-N-(propan-2-yl)-5′,6′-dihydrospiro[pyrrolidine-3,4′-pyrrolo[1,2-b]pyrazole]-1-carboxamide

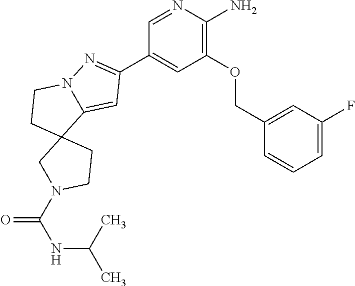 [SMILES: CC(C)NC(=O)N1CCC2(CCn3nc(-c4cnc(N)c(OCc5cccc(F)c5)c4)cc32)C1]

Example 437 was prepared in analogy to

Example 416 using (rac)-5-[5′,6′-dihydrospiro[pyrrolidine-3,4′-pyrrolo[1,2-b]pyrazol]-2′-yl]-3-[(3-fluorophenyl)methoxy]pyridin-2-amine-hydrochloride salt (86.0 mg, 190 μmol) and 2-isocyanatopropane (17.0 mg, 200 μmol).

LC-MS (Method 1): Rt=1.03 min; MS (ESIpos): m/z=465 [M+H]⁺

¹H NMR (400 MHz, DMSO-d₆) δ ppm 1.02-1.11 (m, 6H) 1.99-2.10 (m, 2H) 2.52-2.57 (m, 2H) 3.35-3.53 (m, 4H) 3.76 (dq, 1H) 4.16 (t, 2H) 5.20 (s, 2H) 5.80-5.92 (m, 3H) 6.34 (s, 1H) 7.15 (t, 1H) 7.35-7.47 (m, 4H) 7.94 (d, 1H)

### Example 438

### (rac)-2′-{6-amino-5-[(3-fluorophenyl)methoxy]pyridin-3-yl}-N-cyclobutyl-5′,6′-dihydrospiro[pyrrolidine-3,4′-pyrrolo[1,2-b]pyrazole]-1-carboxamide

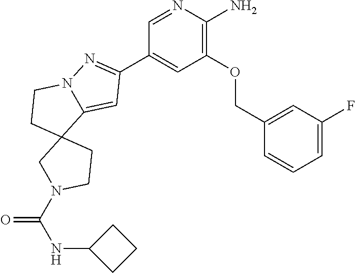 [SMILES: Nc1ncc(-c2cc3n(n2)CCC32CCN(C(=O)NC3CCC3)C2)cc1OCc1cccc(F)c1]

Example 438 was prepared in analogy to

Example 416 using (rac)-5-[5′,6′-dihydrospiro[pyrrolidine-3,4′-pyrrolo[1,2-b]pyrazol]-2′-yl]-3-[(3-fluorophenyl)methoxy]pyridin-2-amine-hydrochloride salt (86.0 mg, 190 μmol) and isocyanatocyclobutane (19.4 mg, 200 μmol).

LC-MS (Method 1): Rt=1.05 min; MS (ESIpos): m/z=477 [M+H]⁺

¹H NMR (400 MHz, DMSO-d₆) δ ppm 1.46-1.62 (m, 2H) 1.87-2.14 (m, 6H) 2.52-2.54 (m, 1H) 3.36-3.53 (m, 4H) 4.07-4.20 (m, 3H) 5.21 (s, 2H) 5.88 (s, 2H) 6.31 (d, 1H) 6.34 (s, 1H) 7.15 (t, 1H) 7.35-7.47 (m, 4H) 7.95 (d, 1H)

### Example 439

### (rac)-2′-{6-amino-5-[(3-fluorophenyl)methoxy]pyridin-3-yl}-N-benzyl-5′,6′-dihydrospiro[pyrrolidine-3,4′-pyrrolo[1,2-b]pyrazole]-1-carboxamide

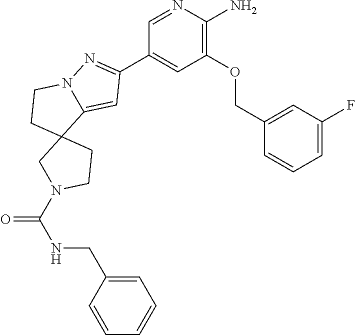 [SMILES: Nc1ncc(-c2cc3n(n2)CCC32CCN(C(=O)NCc3ccccc3)C2)cc1OCc1cccc(F)c1]

Example 439 was prepared in analogy to

Example 416 using (rac)-5-[5′,6′-dihydrospiro[pyrrolidine-3,4′-pyrrolo[1,2-b]pyrazol]-2′-yl]-3-[(3-fluorophenyl)methoxy]pyridin-2-amine-hydrochloride salt (86.0 mg, 190 μmol) and (isocyanatomethyl)benzene (26.6 mg, 200 μmol).

LC-MS (Method 1): Rt=1.10 min; MS (ESIpos): m/z=513 [M+H]⁺

¹H NMR (400 MHz, DMSO-d₆) δ ppm 2.03-2.14 (m, 2H) 2.53-2.56 (m, 2H) 3.38-3.59 (m, 4H) 4.17 (t, 2H) 4.26 (d, 2H) 5.21 (s, 2H) 5.89 (s, 2H) 6.34 (s, 1H) 6.80 (t, 1H) 7.12-7.24 (m, 2H) 7.27-7.33 (m, 4H) 7.35-7.39 (m, 1H) 7.40-7.47 (m, 3H) 7.94 (d, 1H)

### Example 440

### (rac)-2′-{6-amino-5-[(pyridin-2-yl)methoxy]pyridin-3-yl}-N-ethyl-5′,6′-dihydrospiro[pyrrolidine-3,4′-pyrrolo[1,2-b]pyrazole]-1-carboxamide

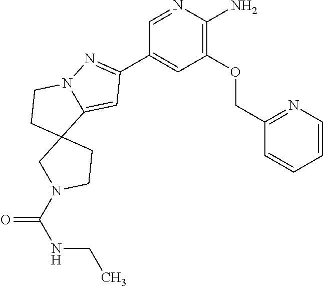 [SMILES: CCNC(=O)N1CCC2(CCn3nc(-c4cnc(N)c(OCc5ccccn5)c4)cc32)C1]

Example 440 was prepared in analogy to

Example 416 using (rac)-5-[5′,6′-dihydrospiro[pyrrolidine-3,4′-pyrrolo[1,2-b]pyrazol]-2′-yl]-3-[(pyridin-2-yl)methoxy]pyridin-2-amine-hydrochloride salt (106 mg, 225 μmol) and isocyanatoethane (19 μL, 240 μmol).

LC-MS (Method 1): Rt=0.79 min; MS (ESIpos): m/z=434 [M+H]⁺

¹H NMR (400 MHz, DMSO-d₆) δ ppm 1.02 (t, 3H) 1.99-2.10 (m, 2H) 2.52-2.56 (m, 2H) 3.01-3.09 (m, 2H) 3.35-3.52 (m, 4H) 4.12-4.22 (m, 2H) 5.24 (s, 2H) 5.90 (s, 2H) 6.15 (t, 1H) 6.34 (s, 1H) 7.35 (ddd, 1H) 7.41 (d, 1H) 7.67 (d, 1H) 7.84 (td, 1H) 7.95 (d, 1H) 8.58 (d, 1H)

### Example 441

### (rac)-2′-{6-amino-5-[(pyridin-2-yl)methoxy]pyridin-3-yl}-N-(propan-2-yl)-5′,6′-dihydrospiro[pyrrolidine-3,4′-pyrrolo[1,2-b]pyrazole]-1-carboxamide

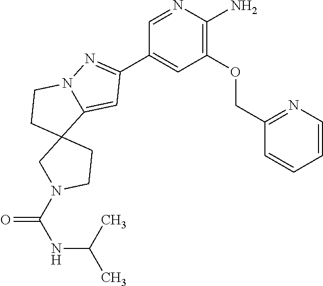 [SMILES: CC(C)NC(=O)N1CCC2(CCn3nc(-c4cnc(N)c(OCc5ccccn5)c4)cc32)C1]

Example 441 was prepared in analogy to

Example 416 using (rac)-5-[5′,6′-dihydrospiro[pyrrolidine-3,4′-pyrrolo[1,2-b]pyrazol]-2′-yl]-3-[(pyridin-2-yl)methoxy]pyridin-2-amine-hydrochloride salt (106 mg, 225 μmol) and 2-isocyanatopropane (20.1 mg, 236 μmol).

LC-MS (Method 1): Rt=0.85 min; MS (ESIpos): m/z=448 [M+H]⁺

¹H NMR (400 MHz, DMSO-d₆) δ ppm 1.06 (br d, 3H) 1.07 (br d, 3H) 1.98-2.09 (m, 2H) 2.52-2.56 (m, 2H) 3.35-3.43 (m, 3H) 3.45-3.57 (m, 1H) 3.70-3.81 (m, 1H) 4.15 (t, 2H) 5.24 (s, 2H) 5.82 (d, 1H) 5.90 (s, 2H) 6.34 (s, 1H) 7.32-7.43 (m, 2H) 7.67 (d, 1H) 7.84 (td, 1H) 7.95 (d, 1H) 8.58 (d, 1H)

### Example 442

### (rac)-2′-{6-amino-5-[(pyridin-2-yl)methoxy]pyridin-3-yl}-N-cyclobutyl-5′,6′-dihydrospiro[pyrrolidine-3,4′-pyrrolo[1,2-b]pyrazole]-1-carboxamide

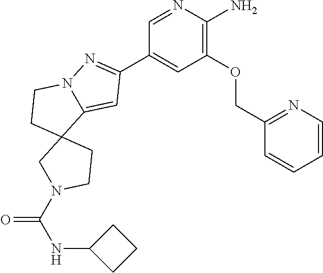 [SMILES: Nc1ncc(-c2cc3n(n2)CCC32CCN(C(=O)NC3CCC3)C2)cc1OCc1ccccn1]

Example 442 was prepared in analogy to

Example 416 using (rac)-5-[5′,6′-dihydrospiro[pyrrolidine-3,4′-pyrrolo[1,2-b]pyrazol]-2′-yl]-3-[(pyridin-2-yl)methoxy]pyridin-2-amine-hydrochloride salt (106 mg, 225 μmol) and isocyanatocyclobutane (22.9 mg, 236 μmol).

LC-MS (Method 1): Rt=0.87 min; MS (ESIpos): m/z=460 [M+H]⁺

¹H NMR (400 MHz, DMSO-d₆) δ ppm 1.46-1.62 (m, 2H) 1.87-2.13 (m, 6H) 2.52-2.56 (m, 2H) 3.35-3.52 (m, 4H) 4.06-4.22 (m, 3H) 5.24 (s, 2H) 5.90 (s, 2H) 6.31 (d, 1H) 6.34 (s, 1H) 7.32-7.44 (m, 2H) 7.67 (d, 1H) 7.84 (td, 1H) 7.95 (d, 1H) 8.58 (d, 1H)

### Example 443

### (rac)-2′-{6-amino-5-[(2-chloro-5-fluorophenyl)methoxy]pyridin-3-yl}-N-ethyl-5′,6′-dihydrospiro[pyrrolidine-3,4′-pyrrolo[1,2-b]pyrazole]-1-carboxamide

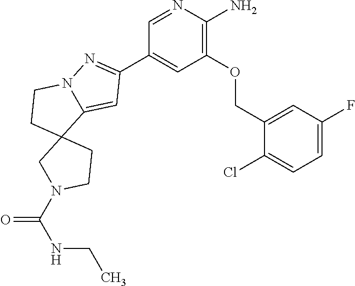 [SMILES: CCNC(=O)N1CCC2(CCn3nc(-c4cnc(N)c(OCc5cc(F)ccc5Cl)c4)cc32)C1]

Example 443 was prepared in analogy to

Example 416 using (rac)-3-[(2-chloro-5-fluorophenyl)methoxy]-5-[5′,6′-dihydrospiro[pyrrolidine-3,4′-pyrrolo[1,2-b]pyrazol]-2′-yl]pyridin-2-amine-hydrochloride salt (80.0 mg, 164 μmol) and isocyanatoethane (14 μL, 170 μmol).

LC-MS (Method 1): Rt=1.03 min; MS (ESIpos): m/z=485 [M+H]⁺

¹H NMR (400 MHz, DMSO-d₆) δ ppm 1.02 (t, 5H) 2.00-2.11 (m, 3H) 2.52-2.56 (m, 2H) 3.01-3.10 (m, 3H) 4.18 (t, 3H) 5.22 (s, 3H) 6.13-6.19 (m, 1H) 6.39 (s, 1H) 7.27 (td, 1H) 7.51 (d, 1H) 7.57 (dd, 1H) 7.72 (dd, 1H) 7.97 (d, 1H)

### Example 444

### (rac)-2′-{6-amino-5-[(2-chloro-5-fluorophenyl)methoxy]pyridin-3-yl}-N-(propan-2-yl)-5′,6′-dihydrospiro[pyrrolidine-3,4′-pyrrolo[1,2-b]pyrazole]-1-carboxamide

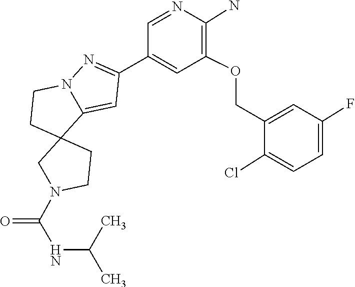

Example 444 was prepared in analogy to

Example 416 using (rac)-3-[(2-chloro-5-fluorophenyl)methoxy]-5-[5′,6′-dihydrospiro[pyrrolidine-3,4′-pyrrolo[1,2-b]pyrazol]-2′-yl]pyridin-2-amine-hydrochloride salt (80.0 mg, 164 μmol) and 2-isocyanatopropane (14.7 mg, 173 μmol).

LC-MS (Method 1): Rt=1.09 min; MS (ESIpos): m/z=499 [M+H]⁺

¹H NMR (400 MHz, DMSO-d₆) δ ppm 1.06 (br d, 3H) 1.07 (br d, 3H) 1.98-2.11 (m, 2H) 2.52-2.54 (m, 2H) 3.36-3.58 (m, 4H) 3.71-3.81 (m, 1H) 4.17 (t, 2H) 5.21 (s, 2H) 5.82 (d, 1H) 6.02 (br s, 2H) 6.37 (s, 1H) 7.27 (td, 1H) 7.47 (d, 1H) 7.57 (dd, 1H) 7.72 (dd, 1H) 7.97 (d, 1H)

### Example 445

### 2′-{6-amino-5-[(1R)-1-(pyridin-2-yl)ethoxy]pyridin-3-yl}-N-(propan-2-yl)-5′,6′-dihydrospiro[pyrrolidine-3,4′-pyrrolo[1,2-b]pyrazole]-1-carboxamide (Mixture of Stereoisomers)

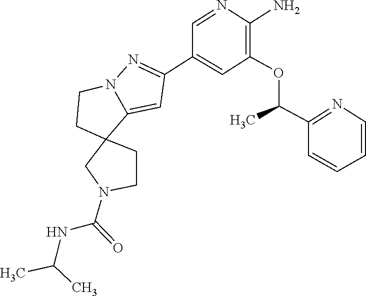 [SMILES: CC(C)NC(=O)N1CCC2(CCn3nc(-c4cnc(N)c(O[C@H](C)c5ccccn5)c4)cc32)C1]

Example 445 was prepared in analogy to

Example 416 using (rac)-5-[5′,6′-dihydrospiro[pyrrolidine-3,4′-pyrrolo[1,2-b]pyrazol]-2′-yl]-3-[(1R)-1-(pyridin-2-yl)ethoxy]pyridin-2-amine-hydrochloride salt (50.0 mg, 121 μmol, diastereomeric mixture) and 2-isocyanatopropane (12 μL, 120 μmol).

LC-MS (method 1): Rₜ=0.89 min; MS (ESIpos): m/z=463 [M+H]⁺

¹H-NMR (400 MHz, DMSO-d6) δ [ppm]: 0.995 (0.76), 1.012 (0.81), 1.022 (0.40), 1.034 (0.64), 1.047 (11.90), 1.050 (12.63), 1.057 (14.52), 1.059 (15.07), 1.064 (14.31), 1.066 (13.81), 1.074 (13.59), 1.609 (16.00), 1.625 (15.96), 1.984 (1.18), 1.997 (2.26), 2.003 (2.46), 2.015 (3.00), 2.021 (2.69), 2.033 (1.32), 2.039 (1.08), 2.052 (0.53), 2.460 (3.18), 2.479 (5.83), 2.518 (2.93), 2.522 (1.80), 3.368 (10.79), 3.372 (11.23), 3.397 (1.03), 3.451 (1.10), 3.469 (1.91), 3.477 (1.25), 3.483 (1.47), 3.491 (1.18), 3.495 (1.18), 3.509 (0.75), 3.725 (0.87), 3.730 (1.06), 3.746 (1.66), 3.762 (1.66), 3.778 (1.04), 3.783 (0.88), 4.103 (3.96), 4.120 (7.01), 4.138 (3.93), 5.490 (0.99), 5.506 (3.73), 5.522 (3.76), 5.538 (1.02), 5.798 (3.33), 5.817 (3.28), 5.892 (9.98), 6.213 (10.52), 6.216 (11.00), 7.200 (3.62), 7.206 (6.03), 7.211 (3.81), 7.269 (1.33), 7.272 (1.62), 7.275 (1.60), 7.277 (1.57), 7.282 (1.55), 7.284 (1.82), 7.288 (2.54), 7.290 (2.59), 7.293 (1.90), 7.296 (1.72), 7.300 (1.74), 7.303 (1.78), 7.305 (1.72), 7.308 (1.61), 7.490 (2.67), 7.492 (2.82), 7.494 (2.74), 7.509 (3.07), 7.512 (3.27), 7.514 (3.08), 7.763 (1.52), 7.766 (2.21), 7.770 (1.68), 7.782 (2.41), 7.785 (3.74), 7.789 (2.62), 7.801 (1.30), 7.805 (1.80), 7.809 (1.25), 7.864 (7.71), 7.868 (7.99), 8.537 (1.71), 8.541 (3.17), 8.544 (3.17), 8.546 (2.09), 8.549 (2.07), 8.551 (3.10), 8.554 (3.17), 8.558 (1.79).

### Example 446

### 2′-{6-amino-5-[(1R)-1-(pyridin-2-yl)ethoxy]pyridin-3-yl}-N-ethyl-5′,6′-dihydrospiro[pyrrolidine-3,4′-pyrrolo[1,2-b]pyrazole]-1-carboxamide (Mixture of Stereoisomers)

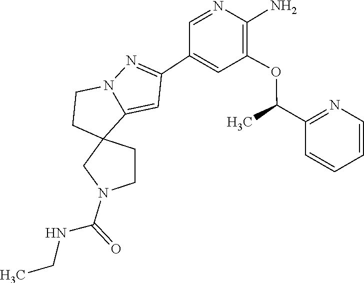 [SMILES: CCNC(=O)N1CCC2(CCn3nc(-c4cnc(N)c(O[C@H](C)c5ccccn5)c4)cc32)C1]

Example 446 was prepared in analogy to

Example 416 using (rac)-5-[5′,6′-dihydrospiro[pyrrolidine-3,4′-pyrrolo[1,2-b]pyrazol]-2′-yl]-3-[(1R)-1-(pyridin-2-yl)ethoxy]pyridin-2-amine-hydrochloride salt (50.0 mg, 121 μmol, diastereomeric mixture) and isocyanatoethane (9.9 μL, 120 μmol).

LC-MS (method 1): Rₜ=0.83 min; MS (ESIpos): m/z=449 [M+H]⁺

¹H-NMR (400 MHz, DMSO-d6) δ [ppm]: 0.997 (6.34), 1.000 (6.79), 1.015 (14.29), 1.018 (14.57), 1.033 (7.01), 1.035 (6.94), 1.137 (0.70), 1.154 (0.74), 1.589 (0.72), 1.609 (15.35), 1.626 (15.58), 2.009 (2.73), 2.021 (3.20), 2.026 (3.01), 2.038 (1.52), 2.459 (3.50), 2.478 (6.13), 3.015 (0.89), 3.018 (0.97), 3.032 (3.32), 3.036 (2.99), 3.050 (4.93), 3.064 (3.00), 3.068 (3.29), 3.082 (1.01), 3.086 (0.90), 3.343 (16.00), 3.365 (11.75), 3.398 (1.45), 3.442 (1.19), 3.460 (2.03), 3.467 (1.38), 3.474 (1.56), 3.481 (1.31), 3.499 (0.77), 4.102 (3.98), 4.120 (6.86), 4.137 (4.05), 5.495 (1.02), 5.512 (3.71), 5.528 (3.74), 5.544 (1.08), 5.913 (5.18), 6.131 (1.80), 6.145 (3.59), 6.159 (1.84), 6.215 (9.05), 6.218 (9.47), 7.209 (3.98), 7.214 (6.86), 7.218 (4.13), 7.269 (1.36), 7.273 (1.82), 7.276 (1.67), 7.281 (1.64), 7.288 (2.97), 7.295 (1.85), 7.299 (1.79), 7.304 (2.08), 7.307 (1.70), 7.491 (3.58), 7.494 (3.65), 7.512 (4.15), 7.514 (4.10), 7.765 (1.96), 7.767 (2.02), 7.769 (1.83), 7.787 (3.44), 7.804 (1.64), 7.806 (1.65), 7.868 (6.80), 7.871 (6.93), 8.539 (3.38), 8.542 (3.79), 8.544 (3.63), 8.552 (3.44), 8.554 (3.66), 8.556 (3.33).

### Example 447

### 2′-{6-amino-5-[(1S)-1-(pyridin-2-yl)ethoxy]pyridin-3-yl}-N-(propan-2-yl)-5′,6′-dihydrospiro[pyrrolidine-3,4′-pyrrolo[1,2-b]pyrazole]-1-carboxamide (Mixture of Stereoisomers)

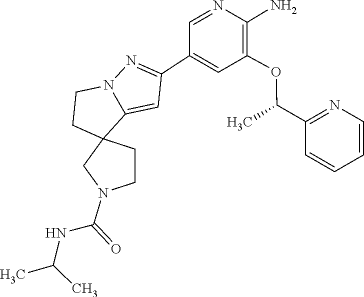 [SMILES: CC(C)NC(=O)N1CCC2(CCn3nc(-c4cnc(N)c(O[C@@H](C)c5ccccn5)c4)cc32)C1]

Example 447 was prepared in analogy to

Example 416 using (rac)-5-[5′,6′-dihydrospiro[pyrrolidine-3,4′-pyrrolo[1,2-b]pyrazol]-2′-yl]-3-[(1S)-1-(pyridin-2-yl)ethoxy]pyridin-2-amine-hydrochloride salt (65.0 mg, 157 μmol, diastereomeric mixture) and 2-isocyanatopropane (15 μL, 160 μmol).

LC-MS (method 1): Rₜ=0.89 min; MS (ESIpos): m/z=463 [M+H]⁺

¹H-NMR (400 MHz, DMSO-d6) δ [ppm]: 0.995 (0.99), 1.012 (1.06), 1.022 (0.44), 1.034 (0.71), 1.047 (12.32), 1.050 (12.96), 1.059 (15.65), 1.064 (14.76), 1.066 (13.86), 1.074 (13.98), 1.609 (16.00), 1.625 (15.88), 1.984 (1.26), 1.997 (2.40), 2.003 (2.59), 2.015 (3.11), 2.021 (2.82), 2.033 (1.36), 2.039 (1.12), 2.051 (0.55), 2.460 (3.28), 2.479 (6.16), 2.518 (3.10), 2.522 (1.92), 3.368 (11.29), 3.372 (11.61), 3.397 (1.12), 3.451 (1.16), 3.469 (1.97), 3.477 (1.31), 3.483 (1.52), 3.491 (1.22), 3.495 (1.24), 3.509 (0.76), 3.730 (1.09), 3.746 (1.72), 3.762 (1.69), 3.778 (1.05), 4.103 (4.08), 4.120 (7.21), 4.138 (3.98), 5.198 (0.45), 5.490 (1.04), 5.506 (3.77), 5.522 (3.80), 5.539 (1.06), 5.798 (3.44), 5.817 (3.37), 5.892 (10.26), 6.213 (10.18), 6.216 (10.73), 7.200 (3.72), 7.206 (6.17), 7.211 (3.91), 7.270 (1.32), 7.272 (1.61), 7.275 (1.64), 7.277 (1.56), 7.282 (1.55), 7.284 (1.84), 7.290 (2.64), 7.293 (1.93), 7.296 (1.72), 7.300 (1.69), 7.303 (1.81), 7.305 (1.77), 7.308 (1.60), 7.490 (2.74), 7.492 (2.92), 7.494 (2.81), 7.509 (3.13), 7.512 (3.36), 7.514 (3.16), 7.763 (1.51), 7.766 (2.28), 7.770 (1.66), 7.782 (2.53), 7.785 (3.80), 7.789 (2.67), 7.801 (1.29), 7.805 (1.83), 7.809 (1.26), 7.863 (7.74), 7.868 (7.96), 8.537 (1.77), 8.541 (3.31), 8.546 (2.17), 8.549 (2.17), 8.554 (3.30).

### Example 448

### 2′-{6-amino-5-[(1S)-1-(pyridin-2-yl)ethoxy]pyridin-3-yl}-N-ethyl-5′,6′-dihydrospiro[pyrrolidine-3,4′-pyrrolo[1,2-b]pyrazole]-1-carboxamide (Mixture of Stereoisomers)

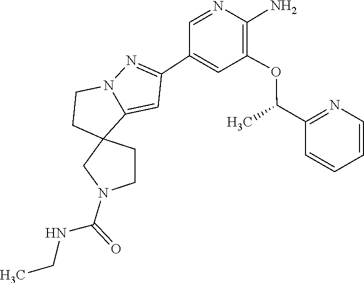 [SMILES: CCNC(=O)N1CCC2(CCn3nc(-c4cnc(N)c(O[C@@H](C)c5ccccn5)c4)cc32)C1]

Example 448 was prepared in analogy to

Example 416 using (rac)-5-[5′,6′-dihydrospiro[pyrrolidine-3,4′-pyrrolo[1,2-b]pyrazol]-2′-yl]-3-[(1S)-1-(pyridin-2-yl)ethoxy]pyridin-2-amine-hydrochloride salt (65.0 mg, 157 μmol, diastereomeric mixture) and isocyanatoethane (13 μL, 160 μmol).

LC-MS (method 1): Rₜ=0.83 min; MS (ESIpos): m/z=449 [M+H]⁺

¹H-NMR (400 MHz, DMSO-d6) δ [ppm]: 0.997 (6.32), 1.000 (6.65), 1.015 (14.84), 1.018 (14.39), 1.033 (6.91), 1.036 (6.63), 1.587 (0.71), 1.609 (15.81), 1.625 (16.00), 1.991 (1.11), 2.009 (2.33), 2.021 (2.79), 2.026 (2.63), 2.039 (1.26), 2.045 (1.02), 2.057 (0.45), 2.460 (3.23), 2.479 (5.77), 2.518 (2.12), 2.523 (1.36), 3.014 (0.79), 3.018 (0.87), 3.032 (3.12), 3.036 (2.73), 3.046 (2.94), 3.050 (4.69), 3.053 (2.95), 3.064 (2.70), 3.067 (3.05), 3.081 (0.83), 3.085 (0.75), 3.319 (0.50), 3.365 (9.75), 3.369 (10.19), 3.379 (2.75), 3.398 (1.01), 3.441 (1.06), 3.459 (1.81), 3.466 (1.13), 3.473 (1.35), 3.480 (1.08), 3.485 (1.10), 3.499 (0.66), 4.102 (3.87), 4.120 (6.55), 4.137 (3.79), 5.492 (0.98), 5.508 (3.70), 5.525 (3.67), 5.541 (1.01), 5.891 (9.60), 6.129 (1.69), 6.143 (3.42), 6.156 (1.67), 6.212 (10.16), 6.216 (10.34), 7.204 (3.60), 7.209 (6.39), 7.213 (3.71), 7.270 (1.38), 7.273 (1.76), 7.277 (1.59), 7.282 (1.49), 7.285 (1.96), 7.289 (2.70), 7.291 (2.10), 7.296 (1.59), 7.300 (1.60), 7.305 (1.90), 7.308 (1.55), 7.491 (3.60), 7.494 (3.60), 7.511 (4.04), 7.513 (3.95), 7.763 (1.61), 7.765 (1.88), 7.768 (1.84), 7.770 (1.72), 7.784 (3.09), 7.787 (3.08), 7.802 (1.34), 7.804 (1.53), 7.806 (1.44), 7.808 (1.32), 7.865 (6.13), 7.867 (6.82), 7.870 (6.80), 7.872 (6.21), 8.539 (3.17), 8.542 (3.68), 8.544 (3.40), 8.551 (3.18), 8.554 (3.55), 8.556 (3.12).

### Example 449

### 2′-(6-amino-5-{[1-(5-chloro-3-fluoropyridin-2-yl)ethyl]oxy}pyridin-3-yl)-N-ethyl-5′,6′-dihydrospiro[pyrrolidine-3,4′-pyrrolo[1,2-b]pyrazole]-1-carboxamide (Diastereomeric Mixture)

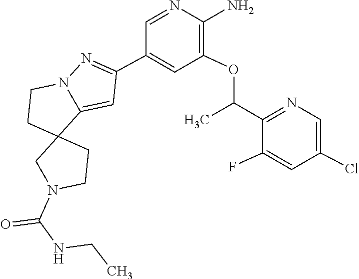 [SMILES: CCNC(=O)N1CCC2(CCn3nc(-c4cnc(N)c(OC(C)c5ncc(Cl)cc5F)c4)cc32)C1]

Example 449 was prepared in analogy to

Example 416 using 3-{[1-(5-chloro-3-fluoropyridin-2-yl)ethyl]oxy}-5-[5′,6′-dihydrospiro[pyrrolidine-3,4′-pyrrolo[1,2-b]pyrazol]-2′-yl]pyridin-2-amine-hydrochloride salt (82.0 mg, 163 μmol, diastereomeric mixture) and isocyanatoethane (14 μL, 172 μmol).

LC-MS (Method 1): Rt=0.96 min; MS (ESIpos): m/z=500 [M+H]⁺

¹H NMR (400 MHz, DMSO-d₆) δ ppm 1.02 (t, 3H) 1.17-1.26 (m, 2H) 1.74 (d, 3H) 2.05 (t, 2H) 2.52-2.56 (m, 3H) 2.99-3.11 (m, 2H) 3.42-3.51 (m, 2H) 4.19 (t, 2H) 6.04-6.20 (m, 2H) 6.44 (s, 1H) 7.64-7.76 (m, 1H) 7.80 (br dd, 1H) 7.87 (s, 1H) 8.13-8.21 (m, 1H) 8.57 (s, 1H)

### Example 450

### (rac)-2′-[6-amino-5-(benzyloxy)pyridin-3-yl]-N-cyclobutyl-5′,6′-dihydrospiro[pyrrolidine-3,4′-pyrrolo[1,2-b]pyrazole]-1-carboxamide

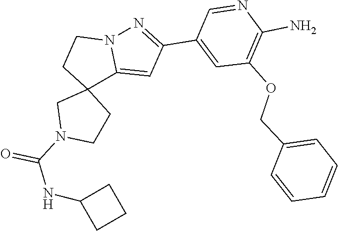 [SMILES: Nc1ncc(-c2cc3n(n2)CCC32CCN(C(=O)NC3CCC3)C2)cc1OCc1ccccc1]

Example 450 was prepared in analogy to

Example 416 using (rac)-3-(benzyloxy)-5-[5′,6′-dihydrospiro[pyrrolidine-3,4′-pyrrolo[1,2-b]pyrazol]-2′-yl]pyridin-2-amine-hydrochloride salt (150 mg, 50% purity, 173 μmol) and isocyanatocyclobutane (17.6 mg, 181 μmol).

¹H-NMR (400 MHz, DMSO-d6): δ [ppm]=1.45-1.62 (m, 2H), 1.86-2.15 (m, 6H), 3.36-3.44 (m, 3H), 3.46-3.54 (m, 1H), 4.06-4.19 (m, 3H), 5.18 (s, 2H), 5.80 (s, 2H), 6.31 (d, 1H), 6.34 (s, 1H), 7.30-7.35 (m, 1H), 7.37-7.42 (m, 2H), 7.43 (d, 1H), 7.50-7.54 (m, 2H), 7.94 (d, 1H).

LC-MS (Method 1): Rt=1.03 min; MS (ESIpos): m/z=459 [M+H]⁺

### Example 451

### 2′-{6-amino-5-[(pyridazin-3-yl)methoxy]pyridin-3-yl}-N-ethyl-5′,6′-dihydrospiro[pyrrolidine-3,4′-pyrrolo[1,2-b]pyrazole]-1-carboxamide (Stereoisomer 2)

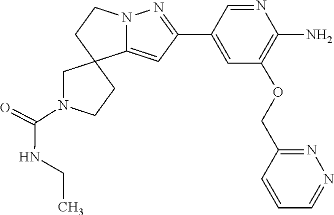 [SMILES: CCNC(=O)N1CCC2(CCn3nc(-c4cnc(N)c(OCc5cccnn5)c4)cc32)C1]

Example 451 was prepared in analogy to

Example 416 using 5-[5′,6′-dihydrospiro[pyrrolidine-3,4′-pyrrolo[1,2-b]pyrazol]-2′-yl]-3-[(pyridazin-3-yl)methoxy]pyridin-2-amine-hydrochloride salt (205 mg, 50% purity, 235 μmol, stereoisomer 2) and isocyanatoethane (20 μL, 250 μmol).

1H-NMR (400 MHz, DMSO-d6): δ [ppm]=1.02 (t, 3H), 1.99-2.11 (m, 2H), 3.01-3.09 (m, 2H), 3.36-3.44 (m, 3H), 3.46-3.53 (m, 1H), 4.16 (t, 2H), 5.47 (s, 2H), 5.98 (s, 2H), 6.15 (t, 1H), 6.37 (s, 1H), 7.50 (d, 1H), 7.77 (dd, 1H), 7.97 (d, 1H), 8.01 (dd, 1H), 9.22 (dd, 1H).

LC-MS (Method 1): Rt=0.68 min; MS (ESIpos): m/z=435 [M+H]⁺

### Example 452

### 2′-{6-amino-5-[(pyridazin-3-yl)methoxy]pyridin-3-yl}-N-ethyl-5′,6′-dihydrospiro[pyrrolidine-3,4′-pyrrolo[1,2-b]pyrazole]-1-carboxamide (Stereoisomer 1)

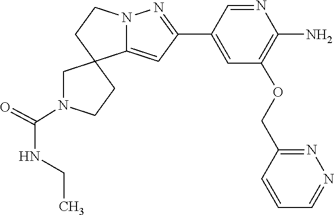 [SMILES: CCNC(=O)N1CCC2(CCn3nc(-c4cnc(N)c(OCc5cccnn5)c4)cc32)C1]

Example 452 was prepared in analogy to

Example 416 using 5-[5′,6′-dihydrospiro[pyrrolidine-3,4′-pyrrolo[1,2-b]pyrazol]-2′-yl]-3-[(pyridazin-3-yl)methoxy]pyridin-2-amine-hydrochloride salt (117 mg, 268 μmol, stereoisomer 1) and isocyanatoethane (22 μL, 280 μmol).

1H-NMR (400 MHz, DMSO-d6): δ [ppm]=1.02 (t, 3H), 1.99-2.11 (m, 2H), 3.02-3.09 (m, 2H), 3.36-3.43 (m, 3H), 3.46-3.53 (m, 1H), 4.16 (t, 2H), 5.47 (s, 2H), 5.98 (br s, 2H), 6.15 (t, 1H), 6.37 (s, 1H), 7.50 (d, 1H), 7.77 (dd, 1H), 7.97 (d, 1H), 8.01 (dd, 1H), 9.22 (dd, 1H).

LC-MS (Method 1): Rt=0.70 min; MS (ESIpos): m/z=435 [M+H]⁺

### Example 453

### (3S)-2′-{6-amino-5-[1-(pyrimidin-5-yl)ethoxy]pyridin-3-yl}-N-ethyl-5′,6′-dihydro-1H-spiro[pyrrolidine-3,4′-pyrrolo[1,2-b]pyrazole]-1-carboxamide (Isomer 1)

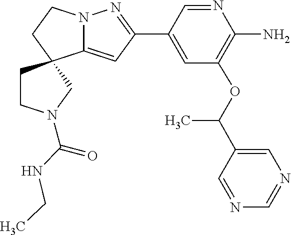 [SMILES: CCNC(=O)N1CC[C@]2(CCn3nc(-c4cnc(N)c(OC(C)c5cncnc5)c4)cc32)C1]

Example 453 was prepared in analogy to

Example 416 using 5-[(3S)-5′,6′-dihydrospiro[pyrrolidine-3,4′-pyrrolo[1,2-b]pyrazol]-2′-yl]-3-{[(1R)-1-(pyrimidin-5-yl)ethyl]oxy}pyridin-2-amine-hydrochloride salt (80.0 mg, 193 μmol, isomer 1) and isocyanatoethane (16 μL, 200 μmol).

¹H NMR (DMSO-d6) δ: 9.12 (s, 1H), 8.99 (s, 2H), 7.91 (d, 1H), 7.42 (d, 1H), 6.31 (s, 1H), 6.15 (t, 1H), 5.96 (s, 2H), 5.79 (q, 1H), 4.14 (t, 2H), 3.35-3.53 (m, 4H), 3.01-3.09 (m, 2H), 1.97-2.10 (m, 2H), 1.62 (d, 3H), 1.02 (t, 3H)

LC-MS (Method 1): Rt=0.73 min; MS (ESIpos): m/z=449 [M+H]⁺

### Example 454

### (3S)-2′-{6-amino-5-[1-(pyrimidin-5-yl)ethoxy]pyridin-3-yl}-N-ethyl-5′,6′-dihydro-1H-spiro[pyrrolidine-3,4′-pyrrolo[1,2-b]pyrazole]-1-carboxamide (Isomer 2)

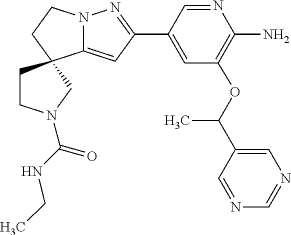 [SMILES: CCNC(=O)N1CC[C@]2(CCn3nc(-c4cnc(N)c(OC(C)c5cncnc5)c4)cc32)C1]

Example 454 was prepared in analogy to

Example 416 using 5-[(3S)-5′,6′-dihydrospiro[pyrrolidine-3,4′-pyrrolo[1,2-b]pyrazol]-2′-yl]-3-{[(1S)-1-(pyrimidin-5-yl)ethyl]oxy}pyridin-2-amine-hydrochloride salt (105 mg, 254 μmol, isomer 3) and isocyanatoethane (21 μL, 270 μmol).

[α]²⁰_{D}: +39.6° (c=1.00, DMSO)

¹H NMR (DMSO-d6) δ: 9.11 (s, 1H), 8.98 (s, 2H), 7.91 (d, 1H), 7.42 (d, 1H), 6.30 (s, 1H), 6.15 (t, 1H), 5.96 (s, 2H), 5.79 (q, 1H), 4.15 (t, 2H), 3.45-3.53 (m, 1H), 3.36-3.43 (m, 3H), 3.01-3.10 (m, 2H), 1.96-2.12 (m, 2H), 1.62 (d, 3H), 1.02 (t, 3H)

### Example 455

### (rac)-2′-(2-amino-1′-methyl-1′,2′,3′,6′-tetrahydro[3,4′-bipyridin]-5-yl)-N-ethyl-5′,6′-dihydrospiro[pyrrolidine-3,4′-pyrrolo[1,2-b]pyrazole]-1-carboxamide

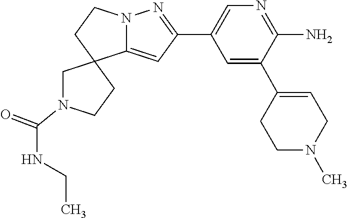 [SMILES: CCNC(=O)N1CCC2(CCn3nc(-c4cnc(N)c(C5=CCN(C)CC5)c4)cc32)C1]

Example 455 was prepared in analogy to

Example 416 using (rac)-5-[5′,6′-dihydrospiro[pyrrolidine-3,4′-pyrrolo[1,2-b]pyrazol]-2′-yl]-1′-methyl-1′,2′,3′,6′-tetrahydro[3,4′-bipyridin]-2-amine-hydrochloride salt (67.0 mg, 173 μmol) and isocyanatoethane (15 μL, 190 μmol).

1H-NMR (400 MHz, CHLOROFORM-d): δ [ppm]=1.17 (t, 3H), 2.03-2.14 (m, 1H), 2.19-2.30 (m, 1H), 2.42 (s, 3H), 2.46-2.52 (m, 2H), 2.57 (s, 1H), 2.61-2.71 (m, 3H), 3.09 (q, 2H), 3.31 (qd, 2H), 3.54 (dt, 3H), 3.60-3.68 (m, 1H), 4.16 (s, 1H), 4.25 (t, 2H), 4.64 (s, 2H), 5.87 (dt, 1H), 6.16 (s, 1H), 7.66 (d, 1H), 8.34 (d, 1H).

### Example 456

### 2′-{6-amino-5-[(1S)-1-(pyridin-2-yl)ethoxy]pyridin-3-yl}-N-ethyl-5′,6′-dihydrospiro[azetidine-3,4′-pyrrolo[1,2-b]pyrazole]-1-carboxamide

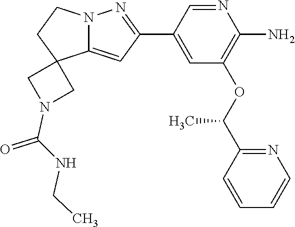 [SMILES: CCNC(=O)N1CC2(CCn3nc(-c4cnc(N)c(O[C@@H](C)c5ccccn5)c4)cc32)C1]

Example 456 was prepared in analogy to

Example 416 using 5-(5′,6′-dihydrospiro[azetidine-3,4′-pyrrolo[1,2-b]pyrazol]-2′-yl)-3-[(1S)-1-(pyridin-2-yl)ethoxy]pyridin-2-amine-hydrochloride salt (14.0 mg, 35.1 μmol) and isocyanatoethane (3.1 μL, 39 μmol).

¹H-NMR (400 MHz, CHLOROFORM-d): δ [ppm]=1.16 (t, 3H), 1.25 (s, 2H), 1.73 (d, 3H), 2.80-2.90 (m, 2H), 3.29 (qd, 2H), 4.06 (dd, 2H), 4.10 (s, 1H), 4.14-4.21 (m, 4H), 4.86 (s, 2H), 5.52 (q, 1H), 6.24 (s, 1H), 7.20 (ddd, 1H), 7.25 (d, 1H), 7.35-7.42 (m, 1H), 7.66 (td, 1H), 8.04 (d, 1H), 8.57-8.63 (m, 1H).

LC-MS (method 1): Rₜ=0.82 min; MS (ESIpos): m/z=434 [M+H]⁺

### Example 457

### 2′-{6-amino-5-[(1S)-1-(pyridin-2-yl)ethoxy]pyridin-3-yl}-N-(propan-2-yl)-5′,6′-dihydrospiro[azetidine-3,4′-pyrrolo[1,2-b]pyrazole]-1-carboxamide

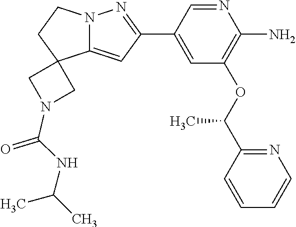 [SMILES: CC(C)NC(=O)N1CC2(CCn3nc(-c4cnc(N)c(O[C@@H](C)c5ccccn5)c4)cc32)C1]

Example 457 was prepared in analogy to

Example 416 using 5-(5′,6′-dihydrospiro[azetidine-3,4′-pyrrolo[1,2-b]pyrazol]-2′-yl)-3-[(1S)-1-(pyridin-2-yl)ethoxy]pyridin-2-amine-hydrochloride salt (14.0 mg, 35.1 μmol) and 2-isocyanatopropane (3.8 μL, 39 μmol).

¹H-NMR (400 MHz, CHLOROFORM-d): δ [ppm]=1.18 (d, 6H), 1.73 (d, 3H), 2.82-2.88 (m, 2H), 3.88-3.94 (m, 1H), 3.94-4.01 (m, 1H), 4.04 (dd, 2H), 4.13-4.21 (m, 4H), 4.86 (s, 2H), 5.52 (q, 1H), 6.24 (s, 1H), 7.20 (ddd, 1H), 7.25 (d, 1H), 7.35-7.41 (m, 1H), 7.66 (td, 1H), 8.05 (d, 1H), 8.58-8.63 (m, 1H).

LC-MS (method 1): Rₜ=0.89 min; MS (ESIpos): m/z=448 [M+H]⁺

### Example 458

### 2′-{6-amino-5-[(1S)-1-phenylethoxy]pyridin-3-yl}-N-ethyl-5′,6′-dihydrospiro[azetidine-3,4′-pyrrolo[1,2-b]pyrazole]-1-carboxamide

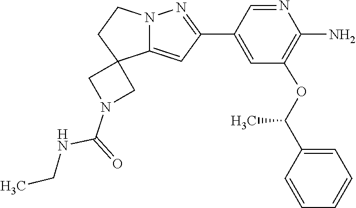 [SMILES: CCNC(=O)N1CC2(CCn3nc(-c4cnc(N)c(O[C@@H](C)c5ccccc5)c4)cc32)C1]

Example 458 was prepared in analogy to

Example 416 using 5-(5′,6′-dihydrospiro[azetidine-3,4′-pyrrolo[1,2-b]pyrazol]-2′-yl)-3-[(1S)-1-phenylethoxy]pyridin-2-amine-hydrochloride salt (98.0 mg, 70% purity, 172 μmol) and isocyanatoethane (13.5 mg, 190 μmol).

1H-NMR (400 MHz, DMSO-d6): δ [ppm]=1.01 (t, 3H), 1.57 (d, 3H), 2.79 (t, 2H), 2.98-3.09 (m, 2H), 3.91-3.98 (m, 4H), 4.06 (t, 2H), 5.60 (q, 1H), 5.85 (s, 2H), 6.43-6.47 (m, 1H), 6.49 (s, 1H), 7.21-7.26 (m, 1H), 7.27 (d, 1H), 7.34 (t, 2H), 7.43-7.51 (m, 2H), 7.88 (d, 1H).

LC-MS (method 1): Rₜ=0.97 min; MS (ESIpos): m/z=434 [M+H]⁺

### Example 459

### 2′-{6-amino-5-[(1R)-1-phenylethoxy]pyridin-3-yl}-N-ethyl-5′,6′-dihydrospiro[azetidine-3,4′-pyrrolo[1,2-b]pyrazole]-1-carboxamide

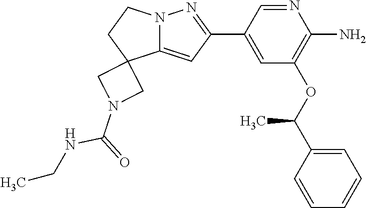 [SMILES: CCNC(=O)N1CC2(CCn3nc(-c4cnc(N)c(O[C@H](C)c5ccccc5)c4)cc32)C1]

Example 459 was prepared in analogy to

Example 416 using 5-(5′,6′-dihydrospiro[azetidine-3,4′-pyrrolo[1,2-b]pyrazol]-2′-yl)-3-[(1R)-1-phenylethoxy]pyridin-2-amine-hydrochloride salt (145 mg, 80% purity, 292 μmol) and isocyanatoethane (22.8 mg, 321 μmol).

1H-NMR (400 MHz, DMSO-d6): δ [ppm]=1.01 (t, 3H), 1.57 (d, 3H), 2.79 (br t, 2H), 2.97-3.08 (m, 2H), 3.89-3.99 (m, 4H), 4.06 (br t, 2H), 5.60 (q, 1H), 5.85 (br s, 2H), 6.45 (t, 1H), 6.49 (s, 1H), 7.19-7.29 (m, 2H), 7.34 (t, 2H), 7.47 (d, 2H), 7.88 (d, 1H).

LC-MS (method 1): Rₜ=0.97 min; MS (ESIpos): m/z=434 [M+H]⁺

### Example 460

### (rac)-2-[6-amino-5-(trifluoromethyl)pyridin-3-yl]-N-[2-(3-chloropyridin-4-yl)propan-2-yl]-6,7-dihydrospiro[pyrazolo[5,1-c][1,4]oxazine-4,3′-pyrrolidine]-1′-carboxamide

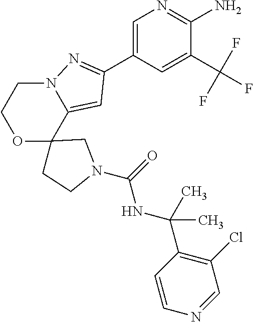 [SMILES: CC(C)(NC(=O)N1CCC2(C1)OCCn1nc(-c3cnc(N)c(C(F)(F)F)c3)cc12)c1ccncc1Cl]

To half of the reaction mixture of 3-chloro-4-(2-isocyanatopropan-2-yl)pyridine (80.0 mg, 407 μmol) was added N,N-diisopropylethylamine (180 μL, 1.0 mmol) and (rac)-5-[6,7-dihydrospiro[pyrazolo[5,1-c][1,4]oxazine-4,3′-pyrrolidin]-2-yl]-3-(trifluoromethyl)pyridin-2-amine hydrochloride (127 mg, 339 μmol). The mixture was stirred at rt overnight under nitrogen atmosphere and then 5 h at 60° C. Saturated potassium carbonate solution was added and stirred for 5 min. The layers were separated and the aqueous phase was extracted three times with dichloromethane (+10% methanol). The combined organic phases were concentrated to give 240 mg of a residue which was purified by flash chromatography to afford 82.0 mg (90% purity, 41% yield) of the title compound.

LC-MS (Method 1): Rt=1.02 min; MS (ESIpos): m/z=536 [M+H]⁺

¹H-NMR (400 MHz, DMSO-d6) δ [ppm]: 1.559 (2.11), 1.648 (16.00), 1.663 (15.19), 2.203 (0.92), 2.227 (1.33), 2.253 (0.72), 2.306 (1.07), 2.323 (1.94), 2.326 (1.92), 2.331 (1.42), 2.354 (0.67), 2.522 (4.29), 2.664 (0.79), 2.669 (1.09), 2.673 (0.81), 3.374 (1.27), 3.393 (1.18), 3.443 (1.85), 3.472 (2.07), 3.542 (0.89), 3.563 (1.53), 3.586 (0.78), 3.747 (1.81), 3.775 (1.55), 4.054 (0.67), 4.070 (1.33), 4.085 (1.61), 4.108 (2.40), 4.120 (4.56), 4.133 (6.73), 6.355 (5.82), 6.601 (7.80), 6.748 (11.16), 7.422 (4.88), 7.436 (5.08), 8.013 (4.62), 8.018 (4.67), 8.397 (7.15), 8.410 (6.34), 8.427 (13.25), 8.582 (4.38), 8.586 (4.30).

### Example 461

### (rac)-2′-[6-amino-5-(trifluoromethyl)pyridin-3-yl]-N-ethyl-5′,6′-dihydrospiro[pyrrolidine-3,4′-pyrrolo[1,2-b]pyrazole]-1-carboxamide

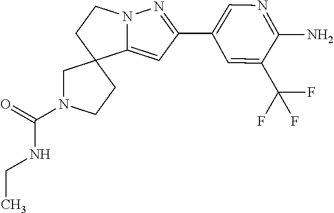 [SMILES: CCNC(=O)N1CCC2(CCn3nc(-c4cnc(N)c(C(F)(F)F)c4)cc32)C1]

(rac)-1-(ethylcarbamoyl)-5′,6′-dihydrospiro[pyrrolidine-3,4′-pyrrolo[1,2-b]pyrazol]-2′-yl trifluoromethanesulfonate (44.2 mg, 116 μmol) was solubilised in 1,4-dioxane (970 μL) under nitrogen, 5-(4,4,5,5-tetramethyl-1,3,2-dioxaborolan-2-yl)-3-(trifluoromethyl)pyridin-2-amine (50.0 mg, 174 μmol), K3PO4 (690 μL, 0.50 M, 350 μmol) and XPhos Pd G2 (45.5 mg, 57.9 μmol) were added. The mixture was stirred overnight at 100° C. It was diluted with ethyl acetate, washed with brine and the organic layer was dried over a silicone filter and concentrated under reduced pressure. The residue was purified by preparative HPLC to give 5.00 mg (95% purity, 10% yield) of the title compound.

LC-MS (Method 1): Rt=0.88 min; MS (ESIneg): m/z=393 [M−H]⁻

¹H NMR (400 MHz, DMSO-d₆) δ ppm 1.02 (t, 3H) 1.99-2.12 (m, 2H) 2.52-2.53 (m, 1H) 2.99-3.09 (m, 2H) 3.36-3.45 (m, 3H) 3.46-3.55 (m, 1H) 4.18 (t, 2H) 6.15 (t, 1H) 6.48 (s, 1H) 6.54 (s, 2H) 8.02 (d, 1H) 8.59 (d, 1H)

### Example 462

### (rac)-2-[6-amino-5-(trifluoromethyl)pyridin-3-yl]-N-ethyl-6,7-dihydro-5H-spiro[pyrazolo[1,5-a]pyridine-4,3′-pyrrolidine]-1′-carboxamide

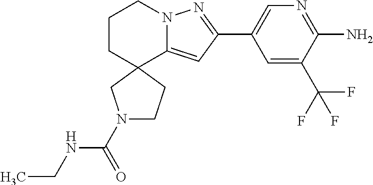 [SMILES: CCNC(=O)N1CCC2(CCCn3nc(-c4cnc(N)c(C(F)(F)F)c4)cc32)C1]

Example 462 was prepared in analogy to Example 461 using (rac)-1′-(ethylcarbamoyl)-6,7-dihydro-5H-spiro[pyrazolo[1,5-a]pyridine-4,3′-pyrrolidin]-2-yl trifluoromethanesulfonate (75.0 mg, 189 μmol) and 5-(4,4,5,5-tetramethyl-1,3,2-dioxaborolan-2-yl)-3-(trifluoromethyl)pyridin-2-amine (81.8 mg, 284 μmol).

LC-MS (Method 1): Rt=0.93 min; MS (ESIpos): m/z=409 [M+H]+

1H NMR (400 MHz, DMSO-d6) δ ppm 1.02 (t, 3H) 1.69-1.85 (m, 2H) 1.94-2.17 (m, 4H) 3.01-3.10 (m, 2H) 3.36-3.42 (m, 3H) 3.44-3.56 (m, 1H) 4.09 (t, 2H) 6.14 (t, 1H) 6.56 (d, 3H) 8.00 (d, 1H) 8.57 (s, 1H)

### Example 463

### (rac)-2′-(6-amino-5-cyanopyridin-3-yl)-N-ethyl-5′,6′-dihydrospiro[pyrrolidine-3,4′-pyrrolo[1,2-b]pyrazole]-1-carboxamide

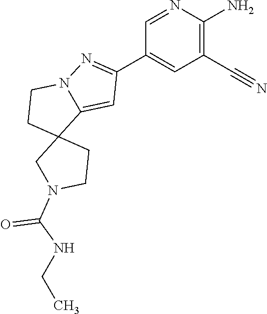 [SMILES: CCNC(=O)N1CCC2(CCn3nc(-c4cnc(N)c(C#N)c4)cc32)C1]

Example 463 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (78.0 mg, 204 μmol) and 2-amino-5-(4,4,5,5-tetramethyl-1,3,2-dioxaborolan-2-yl)pyridine-3-carbonitrile (100 mg, 408 μmol).

¹H NMR (400 MHz, DMSO-d₆) δ ppm 1.02 (t, 3H) 2.05 (t, 2H) 2.52-2.55 (m, 2H) 3.00-3.10 (m, 2H) 3.36-3.52 (m, 4H) 4.18 (t, 2H) 6.15 (t, 1H) 6.45 (s, 1H) 6.98 (s, 2H) 8.15 (d, 1H) 8.62 (d, 1H)

LC-MS (method 1): Rₜ=0.75 min; MS (ESIpos): m/z=352 [M+H]⁺

### Example 464

### (rac)-2′-[6-amino-5-(trifluoromethoxy)pyridin-3-yl]-N-ethyl-5′,6′-dihydrospiro[pyrrolidine-3,4′-pyrrolo[1,2-b]pyrazole]-1-carboxamide

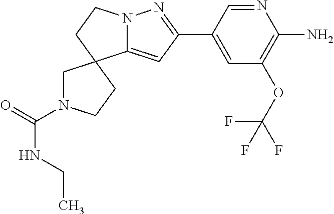 [SMILES: CCNC(=O)N1CCC2(CCn3nc(-c4cnc(N)c(OC(F)(F)F)c4)cc32)C1]

Example 464 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (75.0 mg, 196 μmol) and [6-amino-5-(trifluoromethoxy)pyridin-3-yl]boronic acid (87.1 mg, 75% purity, 294 μmol).

LC-MS (method 2): Rₜ=0.84 min; MS (ESIpos): m/z=412 [M+H]⁺

¹H-NMR (400 MHz, DMSO-d6) δ [ppm]: 1.000 (7.13), 1.018 (16.00), 1.036 (7.33), 1.156 (1.00), 1.232 (0.44), 2.028 (1.30), 2.034 (1.30), 2.042 (1.85), 2.052 (2.56), 2.069 (1.15), 2.082 (0.41), 2.323 (0.52), 2.327 (0.80), 2.332 (0.58), 2.518 (6.18), 2.523 (2.81), 2.533 (2.90), 2.539 (7.17), 2.665 (0.58), 2.669 (0.82), 2.673 (0.55), 3.017 (0.88), 3.035 (2.86), 3.049 (3.13), 3.053 (3.04), 3.067 (2.81), 3.084 (0.83), 3.357 (1.07), 3.375 (1.54), 3.383 (1.34), 3.397 (8.83), 3.419 (0.88), 3.464 (0.74), 3.482 (1.31), 3.488 (0.78), 3.496 (0.91), 3.507 (0.82), 3.521 (0.45), 4.155 (2.64), 4.173 (4.35), 4.190 (2.60), 5.759 (8.19), 6.138 (1.10), 6.152 (2.23), 6.165 (1.10), 6.422 (10.89), 6.494 (6.94), 7.754 (2.56), 7.758 (3.55), 7.762 (2.47), 8.138 (3.41), 8.331 (5.91), 8.336 (5.75).

### Example 465

### (rac)-2′-[6-amino-5-(difluoromethoxy)pyridin-3-yl]-N-ethyl-5′,6′-dihydrospiro[pyrrolidine-3,4′-pyrrolo[1,2-b]pyrazole]-1-carboxamide

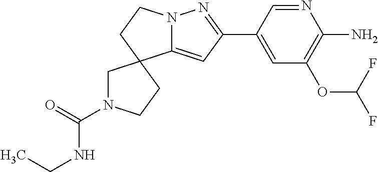 [SMILES: CCNC(=O)N1CCC2(CCn3nc(-c4cnc(N)c(OC(F)F)c4)cc32)C1]

Example 465 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (574 mg, 1.50 mmol) and [6-amino-5-(difluoromethoxy)pyridin-3-yl]boronic acid (352 mg, 1.73 mmol).

1H-NMR (400 MHz, DMSO-d6): δ [ppm]=1.02 (t, 3H), 1.97-2.14 (m, 2H), 3.00-3.10 (m, 2H), 3.36-3.44 (m, 3H), 3.45-3.53 (m, 1H), 4.17 (t, 2H), 6.12-6.19 (m, 3H), 6.37 (s, 1H), 7.18 (t, 1H), 7.59-7.66 (m, 1H), 8.20 (d, 1H).

LC-MS (method 1): Rₜ=0.82 min; MS (ESIpos): m/z=392 [M]⁺

### Example 466

### (rac)-2′-[6-amino-5-(trifluoroacetyl)pyridin-3-yl]-N-ethyl-5′,6′-dihydrospiro[pyrrolidine-3,4′-pyrrolo[1,2-b]pyrazole]-1-carboxamide

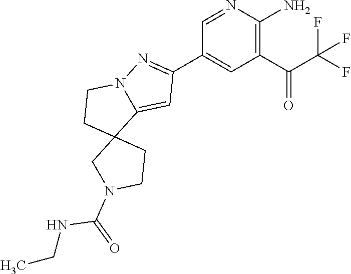 [SMILES: CCNC(=O)N1CCC2(CCn3nc(-c4cnc(N)c(C(=O)C(F)(F)F)c4)cc32)C1]

Example 466 was prepared in analogy to Example 461 using 1-[2-amino-5-(4,4,5,5-tetramethyl-1,3,2-dioxaborolan-2-yl)pyridin-3-yl]-2,2,2-trifluoroethan-1-one (43.0 mg, 136 μmol) and (rac)-1-(ethylcarbamoyl)-5′,6′-dihydrospiro[pyrrolidine-3,4′-pyrrolo[1,2-b]pyrazol]-2′-yl trifluoromethanesulfonate (62.4 mg, 163 μmol).

LC-MS (method 1): Rₜ=0.83 min; MS (ESIpos): m/z=423 [M+H]⁺

¹H-NMR (400 MHz, DMSO-d6) δ [ppm]: 1.002 (6.91), 1.020 (16.00), 1.038 (7.39), 1.227 (1.00), 1.906 (1.12), 2.024 (0.51), 2.041 (1.27), 2.056 (2.24), 2.074 (2.67), 2.093 (1.10), 2.105 (0.66), 2.529 (4.25), 2.548 (2.86), 3.020 (0.89), 3.038 (2.92), 3.052 (3.32), 3.056 (3.24), 3.070 (2.94), 3.088 (0.91), 3.159 (1.41), 3.171 (1.44), 3.363 (1.10), 3.381 (1.39), 3.388 (1.26), 3.400 (1.32), 3.407 (2.76), 3.416 (10.90), 3.474 (0.80), 3.493 (1.30), 3.500 (0.93), 3.506 (1.07), 3.513 (0.85), 3.518 (0.79), 3.532 (0.50), 4.195 (2.67), 4.213 (4.43), 4.230 (2.68), 5.757 (1.99), 6.152 (1.19), 6.166 (2.37), 6.180 (1.21), 6.491 (10.51), 8.149 (4.21), 8.302 (2.25), 8.308 (3.22), 8.313 (2.12), 8.822 (5.27), 8.828 (5.24).

### Example 467

### 2′-(6-amino-5-{[1-phenylethyl]sulfanyl}pyridin-3-yl)-N-ethyl-5′,6′-dihydrospiro[pyrrolidine-3,4′-pyrrolo[1,2-b]pyrazole]-1-carboxamide (Diastereomeric Mixture)

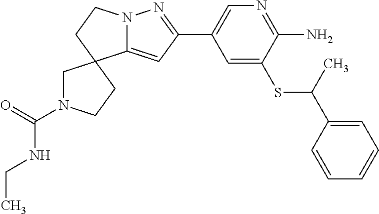 [SMILES: CCNC(=O)N1CCC2(CCn3nc(-c4cnc(N)c(SC(C)c5ccccc5)c4)cc32)C1]

Example 467 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (62.5 mg, 65% purity, 106 μmol) and (rac)-(6-amino-5-{[1-phenylethyl]sulfanyl}pyridin-3-yl)boronic acid (93.0 mg, 47% purity, 159 μmol).

¹H-NMR (400 MHz, DMSO-d6): δ [ppm]=1.03 (t, 3H), 1.52 (d, 3H), 2.00-2.09 (m, 2H), 3.02-3.11 (m, 2H), 3.36-3.43 (m, 3H), 3.45-3.52 (m, 1H), 4.15 (t, 2H), 4.44 (qd, 1H), 6.14-6.24 (m, 4H), 7.18-7.24 (m, 1H), 7.25-7.31 (m, 2H), 7.31-7.36 (m, 2H), 7.64 (d, 1H, diastereomer 1), 7.65 (d, 1H, diastereomer 2), 8.28 (d, 1H).

### Example 468

### (rac)-2′-{6-amino-5-[(cyclohexylmethyl)sulfanyl]pyridin-3-yl}-N-ethyl-5′,6′-dihydrospiro[pyrrolidine-3,4′-pyrrolo[1,2-b]pyrazole]-1-carboxamide

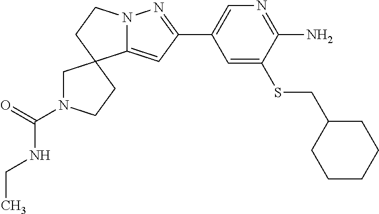 [SMILES: CCNC(=O)N1CCC2(CCn3nc(-c4cnc(N)c(SCC5CCCCC5)c4)cc32)C1]

Example 468 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (194 mg, 60% purity, 302 μmol) and {6-amino-5-[(cyclohexylmethyl)sulfanyl]pyridin-3-yl}boronic acid (214 mg, 45% purity, 362 μmol).

¹H-NMR (500 MHz, DMSO-d6): δ [ppm]=0.93-1.04 (m, 5H), 1.09-1.21 (m, 3H), 1.35-1.44 (m, 1H), 1.55-1.62 (m, 1H), 1.63-1.70 (m, 2H), 1.82 (br d, 2H), 2.00-2.10 (m, 2H), 2.73 (d, 2H), 3.02-3.08 (m, 2H), 3.36-3.42 (m, 3H), 3.47-3.53 (m, 1H), 4.17 (t, 2H), 6.08 (s, 2H), 6.15 (t, 1H), 6.37 (s, 1H), 7.83 (d, 1H), 8.26 (d, 1H).

### Example 469

### (rac)-2′-[6-amino-5-(benzylsulfanyl)pyridin-3-yl]-N-ethyl-5′,6′-dihydrospiro[pyrrolidine-3,4′-pyrrolo[1,2-b]pyrazole]-1-carboxamide

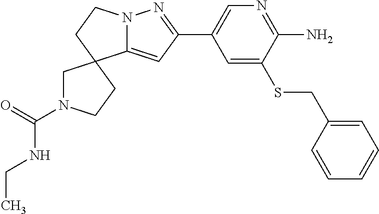 [SMILES: CCNC(=O)N1CCC2(CCn3nc(-c4cnc(N)c(SCc5ccccc5)c4)cc32)C1]

Example 469 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (83.5 mg, 92% purity, 201 μmol) and 3-(benzylsulfanyl)-5-(4,4,5,5-tetramethyl-1,3,2-dioxaborolan-2-yl)pyridin-2-amine (220 mg, 38% purity, 241 μmol).

¹H-NMR (400 MHz, DMSO-d6): δ [ppm]=1.02 (t, 3H), 1.98-2.10 (m, 2H), 3.01-3.10 (m, 2H), 3.36-3.43 (m, 3H), 3.45-3.53 (m, 1H), 4.07 (s, 2H), 4.15 (t, 2H), 6.11-6.18 (m, 3H), 6.25 (s, 1H), 7.18-7.24 (m, 1H), 7.25-7.28 (m, 4H), 7.68 (d, 1H), 8.26 (d, 1H).

### Example 470

### (rac)-2′-[6-amino-5-(2-hydroxypropan-2-yl)pyridin-3-yl]-N-ethyl-5′,6′-dihydrospiro[pyrrolidine-3,4′-pyrrolo[1,2-b]pyrazole]-1-carboxamide

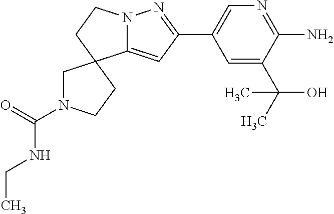 [SMILES: CCNC(=O)N1CCC2(CCn3nc(-c4cnc(N)c(C(C)(C)O)c4)cc32)C1]

Example 470 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (100 mg, 262 μmol) and 2-[2-amino-5-(4,4,5,5-tetramethyl-1,3,2-dioxaborolan-2-yl)pyridin-3-yl]propan-2-ol (115 mg, 76% purity, 314 μmol).

¹H-NMR (400 MHz, DMSO-d6): δ [ppm]=1.02 (t, 3H), 1.51 (s, 6H), 1.98-2.12 (m, 2H), 3.01-3.10 (m, 2H), 3.36-3.43 (m, 3H), 3.46-3.54 (m, 1H), 4.16 (t, 2H), 5.45 (s, 1H), 6.12-6.19 (m, 3H), 6.33 (s, 1H), 7.64 (d, 1H), 8.21 (d, 1H).

### Example 471

### 2′-(6-amino-5-{[1-cyclohexylethyl]sulfanyl}pyridin-3-yl)-N-ethyl-5′,6′-dihydrospiro[pyrrolidine-3,4′-pyrrolo[1,2-b]pyrazole]-1-carboxamide (Diastereomeric Mixture)

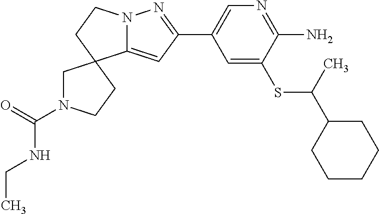 [SMILES: CCNC(=O)N1CCC2(CCn3nc(-c4cnc(N)c(SC(C)C5CCCCC5)c4)cc32)C1]

Example 471 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (70.3 mg, 92% purity, 169 μmol) and (rac)-(6-amino-5-{[1-cyclohexylethyl]sulfanyl}pyridin-3-yl)boronic acid (141 mg, 40% purity, 203 μmol).

¹H NMR (400 MHz, DMSO-d6) δ ppm 1.02 (t, 3H) 1.06-1.25 (m, 8H) 1.39-1.50 (m, 1H) 1.57-1.65 (m, 1H) 1.67-1.77 (m, 3H) 1.78-1.85 (m, 1H) 1.99-2.11 (m, 2H) 3.01-3.14 (m, 3H) 3.36-3.44 (m, 3H) 3.45-3.54 (m, 1H) 4.17 (br t, 2H) 6.11 (s, 2H) 6.15 (br t, 1H) 6.37 (s, 1H) 7.86 (d, 1H) 8.31 (d, 1H)

### Example 472

### 2′-{6-amino-5-[(1R)-1-(pyridin-3-yl)ethoxy]pyridin-3-yl}-N-ethyl-5′,6′-dihydrospiro[pyrrolidine-3,4′-pyrrolo[1,2-b]pyrazole]-1-carboxamide (Mixture of Stereoisomers)

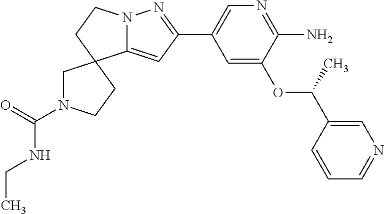 [SMILES: CCNC(=O)N1CCC2(CCn3nc(-c4cnc(N)c(O[C@H](C)c5cccnc5)c4)cc32)C1]

Example 472 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (95.3 mg, 92% purity, 229 μmol) and {6-amino-5-[(1R)-1-(pyridin-3-yl)ethoxy]pyridin-3-yl}boronic acid (168 mg, 39% purity, 252 μmol).

¹H-NMR (400 MHz, DMSO-d6): δ [ppm]=1.02 (2t, 3H), 1.60 (d, 3H), 1.97-2.08 (m, 2H), 3.00-3.10 (m, 2H), 3.36-3.43 (m, 3H), 3.44-3.53 (m, 1H), 4.13 (t, 2H), 5.71 (q, 1H), 5.89 (s, 2H), 6.15 (t, 1H), 6.27 (d, 1H), 7.32 (s, 1H), 7.37 (dd, 1H), 7.87 (t, 1H), 7.91 (dt, 1H), 8.47 (dd, 1H), 8.71 (d, 1H).

### Example 473

### 2′-{6-amino-5-[(1S)-1-(pyridin-3-yl)ethoxy]pyridin-3-yl}-N-ethyl-5′,6′-dihydrospiro[pyrrolidine-3,4′-pyrrolo[1,2-b]pyrazole]-1-carboxamide (Mixture of Stereoisomers)

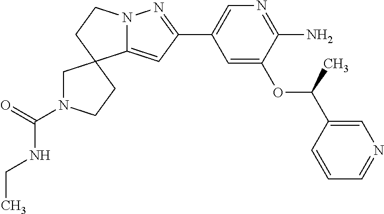 [SMILES: CCNC(=O)N1CCC2(CCn3nc(-c4cnc(N)c(O[C@@H](C)c5cccnc5)c4)cc32)C1]

Example 473 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (138 mg, 92% purity, 333 μmol) and {6-amino-5-[(1S)-1-(pyridin-3-yl)ethoxy]pyridin-3-yl}boronic acid (265 mg, 36% purity, 366 μmol).

¹H-NMR (400 MHz, DMSO-d6): δ [ppm]=1.02 (2t, 3H), 1.60 (d, 3H), 1.96-2.10 (m, 2H), 3.01-3.09 (m, 2H), 3.36-3.43 (m, 3H), 3.44-3.52 (m, 1H), 4.13 (t, 2H), 5.71 (q, 1H), 5.89 (s, 2H), 6.15 (t, 1H), 6.27 (d, 1H), 7.32 (s, 1H), 7.37 (dd, 1H), 7.87 (t, 1H), 7.91 (dt, 1H), 8.47 (dd, 1H), 8.71 (d, 1H).

### Example 474

### (rac)-2′-{6-amino-5-[(cyclopentylmethyl)sulfanyl]pyridin-3-yl}-N-ethyl-5′,6′-dihydrospiro[pyrrolidine-3,4′-pyrrolo[1,2-b]pyrazole]-1-carboxamide

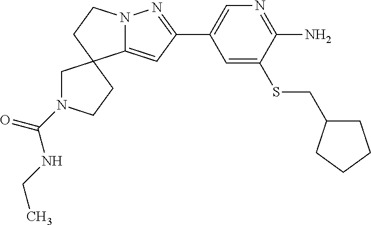 [SMILES: CCNC(=O)N1CCC2(CCn3nc(-c4cnc(N)c(SCC5CCCC5)c4)cc32)C1]

Example 474 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (93.3 mg, 92% purity, 224 μmol) and 3-[(cyclopentylmethyl)sulfanyl]-5-(4,4,5,5-tetramethyl-1,3,2-dioxaborolan-2-yl)pyridin-2-amine (168 mg, 54% purity, 269 μmol).

¹H-NMR (400 MHz, DMSO-d6): δ [ppm]=1.02 (t, 3H), 1.20-1.31 (m, 2H), 1.42-1.64 (m, 4H), 1.70-1.81 (m, 2H), 1.89-2.11 (m, 3H), 2.82 (d, 2H), 3.00-3.10 (m, 2H), 3.36-3.43 (m, 3H), 3.46-3.53 (m, 1H), 4.17 (t, 2H), 6.09 (s, 2H), 6.15 (t, 1H), 6.37 (s, 1H), 7.84 (d, 1H), 8.26 (d, 1H).

### Example 475

### (rac)-2′-(6-amino-5-nitropyridin-3-yl)-N-ethyl-5′,6′-dihydrospiro[pyrrolidine-3,4′-pyrrolo[1,2-b]pyrazole]-1-carboxamide

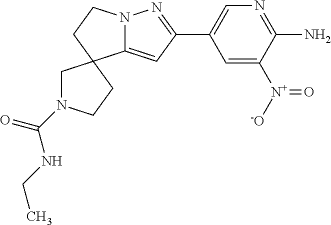

Example 475 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (96.2 mg, 251 μmol) and 3-nitro-5-(4,4,5,5-tetramethyl-1,3,2-dioxaborolan-2-yl)pyridin-2-amine (100 mg, 377 μmol).

¹H NMR (400 MHz, DMSO-d₆) δ ppm 1.02 (t, 3H) 2.03-2.12 (m, 2H) 2.53-2.57 (m, 2H) 3.02-3.09 (m, 2H) 3.37-3.54 (m, 4H) 4.21 (t, 2H) 6.16 (t, 1H) 6.56 (s, 1H) 8.00 (br s, 2H) 8.61 (d, 1H) 8.82 (d, 1H)

### Example 476

### (rac)-2′-[6-amino-5-(benzyloxy)pyridin-3-yl]-N-ethyl-5′,6′-dihydrospiro[pyrrolidine-3,4′-pyrrolo[1,2-b]pyrazole]-1-carboxamide

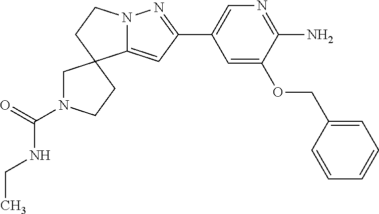 [SMILES: CCNC(=O)N1CCC2(CCn3nc(-c4cnc(N)c(OCc5ccccc5)c4)cc32)C1]

Example 476 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (75.0 mg, 196 μmol) and 3-(benzyloxy)-5-(4,4,5,5-tetramethyl-1,3,2-dioxaborolan-2-yl)pyridin-2-amine (96.0 mg, 294 μmol).

LC-MS (Method 1): Rt=0.97 min; MS (ESIpos): m/z=433 [M+H]⁺

¹H-NMR (400 MHz, DMSO-d6) δ [ppm]: 1.002 (7.23), 1.020 (16.00), 1.038 (7.32), 2.011 (0.42), 2.025 (1.36), 2.040 (2.16), 2.054 (2.82), 2.071 (1.18), 2.083 (0.52), 2.322 (0.66), 2.327 (0.88), 2.331 (0.66), 2.523 (3.16), 2.528 (3.28), 2.539 (1.48), 2.665 (0.67), 2.669 (0.91), 2.673 (0.69), 3.020 (1.00), 3.038 (3.09), 3.052 (3.45), 3.055 (3.36), 3.069 (2.97), 3.087 (0.87), 3.361 (0.69), 3.371 (0.61), 3.379 (1.62), 3.397 (10.81), 3.423 (1.08), 3.465 (0.81), 3.483 (1.41), 3.490 (0.87), 3.497 (1.02), 3.508 (0.85), 3.523 (0.48), 4.139 (2.80), 4.156 (4.68), 4.174 (2.70), 5.182 (11.01), 5.786 (7.12), 6.142 (1.27), 6.155 (2.52), 6.169 (1.24), 6.336 (10.81), 7.306 (0.90), 7.324 (2.97), 7.330 (0.96), 7.343 (2.37), 7.380 (3.87), 7.399 (6.57), 7.417 (3.21), 7.429 (4.75), 7.433 (4.69), 7.512 (5.71), 7.531 (4.35), 7.933 (5.97), 7.938 (5.83).

### Example 477

### (rac)-2′-(6-amino-5-methylpyridin-3-yl)-N-ethyl-5′,6′-dihydrospiro[pyrrolidine-3,4′-pyrrolo[1,2-b]pyrazole]-1-carboxamide

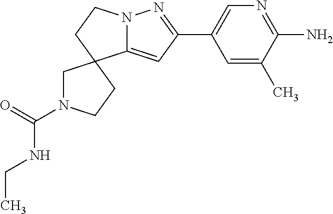 [SMILES: CCNC(=O)N1CCC2(CCn3nc(-c4cnc(N)c(C)c4)cc32)C1]

Example 477 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (75.0 mg, 196 μmol) and 3-methyl-5-(4,4,5,5-tetramethyl-1,3,2-dioxaborolan-2-yl)pyridin-2-amine (68.9 mg, 294 μmol).

LC-MS (Method 1): Rt=0.73 min; MS (ESIpos): m/z=341 [M+H]⁺

¹H-NMR (400 MHz, DMSO-d6) δ [ppm]: 1.000 (5.72), 1.018 (12.97), 1.035 (5.92), 2.005 (0.41), 2.022 (1.17), 2.036 (2.12), 2.062 (16.00), 2.331 (0.52), 2.518 (3.76), 2.522 (4.23), 2.539 (0.52), 2.673 (0.53), 3.017 (0.80), 3.034 (2.51), 3.048 (2.80), 3.052 (2.74), 3.066 (2.44), 3.084 (0.72), 3.365 (1.68), 3.372 (1.28), 3.391 (10.18), 3.408 (0.86), 3.461 (0.69), 3.480 (1.11), 3.487 (0.76), 3.493 (0.86), 3.500 (0.70), 3.506 (0.67), 3.519 (0.43), 4.131 (2.46), 4.149 (4.12), 4.166 (2.38), 5.755 (6.35), 6.136 (1.02), 6.149 (1.99), 6.163 (0.97), 6.283 (8.67), 7.589 (3.04), 7.592 (3.00), 8.172 (3.39), 8.177 (3.29).

### Example 478

### (rac)-2-(6-amino-5-nitropyridin-3-yl)-N-ethyl-6,7-dihydro-5H-spiro[pyrazolo[1,5-a]pyridine-4,3′-pyrrolidine]-1′-carboxamide

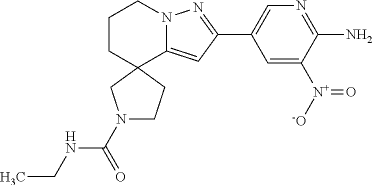

Example 478 was prepared in analogy to Example 461 using (rac)-1′-(ethylcarbamoyl)-6,7-dihydro-5H-spiro[pyrazolo[1,5-a]pyridine-4,3′-pyrrolidin]-2-yl trifluoromethanesulfonate (75.0 mg, 189 μmol) and 3-nitro-5-(4,4,5,5-tetramethyl-1,3,2-dioxaborolan-2-yl)pyridin-2-amine (75.2 mg, 284 μmol).

¹H NMR (400 MHz, DMSO-d₆) δ ppm 1.02 (t, 3H) 1.79 (br d, 2H) 1.95-2.08 (m, 3H) 2.08-2.18 (m, 1H) 3.02-3.09 (m, 2H) 3.38-3.43 (m, 3H) 3.49 (br dd, 1H) 4.11 (t, 2H) 6.14 (t, 1H) 6.65 (s, 1H) 8.01 (br s, 2H) 8.60 (d, 1H) 8.80 (d, 1H)

### Example 479

### methyl 2-amino-5-[(rac)-1-(ethylcarbamoyl)-5′,6′-dihydrospiro[pyrrolidine-3,4′-pyrrolo[1,2-b]pyrazol]-2′-yl]pyridine-3-carboxylate

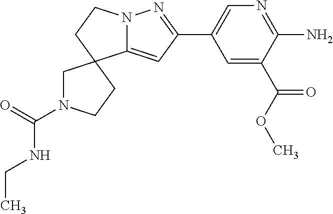 [SMILES: CCNC(=O)N1CCC2(CCn3nc(-c4cnc(N)c(C(=O)OC)c4)cc32)C1]

Example 479 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (75.0 mg, 196 μmol) and methyl 2-amino-5-(4,4,5,5-tetramethyl-1,3,2-dioxaborolan-2-yl)pyridine-3-carboxylate (81.8 mg, 294 μmol).

¹H NMR (400 MHz, DMSO-d₆) δ ppm 1.02 (t, 3H) 2.00-2.12 (m, 2H) 2.52 (br s, 2H) 3.05 (dd, 2H) 3.40 (s, 3H) 3.46-3.57 (m, 1H) 3.84 (s, 3H) 4.18 (t, 2H) 6.16 (t, 1H) 6.41 (s, 1H) 7.24 (s, 2H) 8.39 (d, 1H) 8.61 (d, 1H)

### Example 480

### (rac)-2′-(6-amino-5-phenylpyridin-3-yl)-N-ethyl-5′,6′-dihydrospiro[pyrrolidine-3,4′-pyrrolo[1,2-b]pyrazole]-1-carboxamide

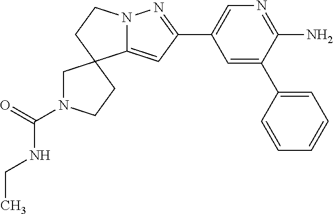 [SMILES: CCNC(=O)N1CCC2(CCn3nc(-c4cnc(N)c(-c5ccccc5)c4)cc32)C1]

Example 480 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (179 mg, 467 μmol) and (6-amino-5-phenylpyridin-3-yl)boronic acid (150 mg, 701 μmol).

¹H NMR (600 MHz, DMSO-d₆) δ ppm 1.02 (t, 3H) 1.99-2.11 (m, 2H) 2.52-2.56 (m, 2H) 3.02-3.09 (m, 2H) 3.36-3.44 (m, 3H) 3.50 (ddd, 1H) 4.14-4.20 (m, 2H) 5.67 (s, 2H) 6.14 (t, 1H) 6.39 (s, 1H) 7.27-7.43 (m, 1H) 7.46-7.52 (m, 4H) 7.66 (d, 1H) 8.36 (d, 1H)

### Example 481

### (rac)-2′-[6-amino-5-(methylsulfanyl)pyridin-3-yl]-N-ethyl-5′,6′-dihydrospiro[pyrrolidine-3,4′-pyrrolo[1,2-b]pyrazole]-1-carboxamide

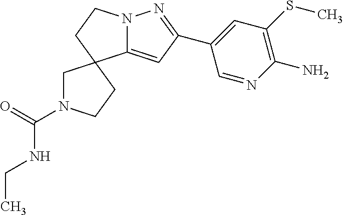 [SMILES: CCNC(=O)N1CCC2(CCn3nc(-c4cnc(N)c(SC)c4)cc32)C1]

Example 481 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (75.0 mg, 196 μmol) and [6-amino-5-(methylsulfanyl)pyridin-3-yl]boronic acid (54.1 mg, 294 μmol).

LC-MS (Method 1): Rt=0.81 min; MS (ESIpos): m/z=373 [M+H]⁺

1H-NMR (400 MHz, DMSO-d6) delta [ppm]: 0.798 (0.43), 0.822 (0.43), 0.905 (0.49), 1.001 (2.76), 1.019 (6.01), 1.035 (9.93), 1.053 (16.00), 1.066 (5.33), 1.070 (8.83), 1.261 (1.72), 1.751 (2.82), 1.864 (1.47), 2.055 (0.92), 2.344 (0.80), 2.411 (13.00), 2.518 (12.93), 2.523 (8.34), 2.564 (0.61), 3.036 (1.04), 3.050 (1.23), 3.067 (1.16), 3.378 (1.72), 3.384 (1.59), 3.392 (2.57), 3.399 (3.37), 3.404 (2.15), 3.417 (1.78), 3.422 (4.05), 3.435 (3.92), 3.439 (3.49), 3.452 (3.49), 3.457 (1.35), 3.469 (1.53), 3.938 (0.80), 4.149 (0.98), 4.168 (1.66), 4.185 (0.86), 4.343 (2.64), 4.355 (5.09), 4.368 (2.45), 4.451 (0.67), 6.001 (2.33), 6.150 (0.80), 6.180 (0.43), 6.380 (3.80), 7.754 (1.72), 7.760 (1.78), 8.220 (1.96), 8.226 (2.08)

### Example 482

### (rac)-2′-[6-amino-5-(4-methylphenyl)pyridin-3-yl]-N-ethyl-5′,6′-dihydrospiro[pyrrolidine-3,4′-pyrrolo[1,2-b]pyrazole]-1-carboxamide

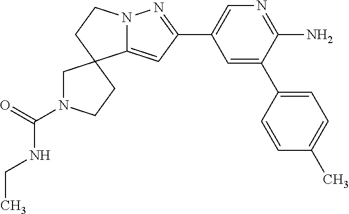 [SMILES: CCNC(=O)N1CCC2(CCn3nc(-c4cnc(N)c(-c5ccc(C)cc5)c4)cc32)C1]

Example 482 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (100 mg, 262 μmol) and 3-(4-methylphenyl)-5-(4,4,5,5-tetramethyl-1,3,2-dioxaborolan-2-yl)pyridin-2-amine (122 mg, 392 μmol).

LC-MS (Method 1): Rt=1.00 min; MS (ESIpos): m/z=417 [M+H]⁺

¹H-NMR (400 MHz, DMSO-d6) δ [ppm]: 0.851 (0.46), 0.995 (4.94), 1.013 (11.11), 1.031 (5.04), 1.232 (2.72), 1.900 (0.43), 2.027 (0.94), 2.040 (1.37), 2.054 (1.83), 2.072 (0.79), 2.084 (0.52), 2.331 (2.84), 2.336 (1.42), 2.358 (12.03), 2.518 (16.00), 2.522 (10.28), 2.539 (7.11), 2.669 (3.88), 2.673 (2.82), 3.012 (0.65), 3.030 (1.98), 3.044 (2.22), 3.048 (2.18), 3.062 (1.95), 3.080 (0.58), 3.367 (1.89), 3.395 (6.80), 3.465 (0.54), 3.483 (0.89), 3.497 (0.70), 4.144 (1.82), 4.162 (3.15), 4.179 (1.76), 5.636 (4.48), 6.134 (0.82), 6.148 (1.68), 6.162 (0.85), 6.382 (7.65), 7.276 (2.85), 7.296 (4.42), 7.361 (5.75), 7.381 (3.39), 7.622 (3.80), 7.627 (3.76), 8.334 (4.08), 8.340 (3.92).

### Example 483

### (rac)-2′-[6-amino-5-(2-methylphenyl)pyridin-3-yl]-N-ethyl-5′,6′-dihydrospiro[pyrrolidine-3,4′-pyrrolo[1,2-b]pyrazole]-1-carboxamide

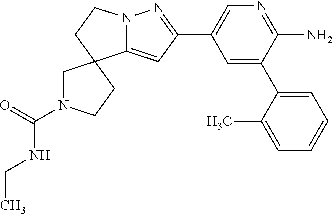 [SMILES: CCNC(=O)N1CCC2(CCn3nc(-c4cnc(N)c(-c5ccccc5C)c4)cc32)C1]

Example 483 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (150 mg, 392 μmol) and 3-(2-methylphenyl)-5-(4,4,5,5-tetramethyl-1,3,2-dioxaborolan-2-yl)pyridin-2-amine (200 mg, 645 μmol).

LC-MS (Method 1): Rt=0.98 min; MS (ESIpos): m/z=417 [M+H]⁺

¹H-NMR (400 MHz, DMSO-d6) δ [ppm]: 0.991 (0.87), 1.009 (1.93), 1.027 (0.91), 2.123 (3.06), 2.518 (0.64), 2.523 (0.72), 2.539 (16.00), 3.041 (0.45), 3.044 (0.44), 3.361 (0.47), 3.387 (1.33), 4.149 (0.66), 5.362 (0.91), 6.371 (1.46), 7.170 (0.42), 7.310 (0.50), 7.313 (0.55), 7.320 (0.57), 7.325 (0.64), 7.329 (0.54), 7.516 (0.73), 7.522 (0.74), 8.375 (0.78), 8.380 (0.77).

### Example 484

### (rac)-2′-[6-amino-5-(3-methylphenyl)pyridin-3-yl]-N-ethyl-5′,6′-dihydrospiro[pyrrolidine-3,4′-pyrrolo[1,2-b]pyrazole]-1-carboxamide

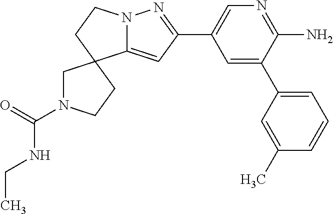 [SMILES: CCNC(=O)N1CCC2(CCn3nc(-c4cnc(N)c(-c5cccc(C)c5)c4)cc32)C1]

Example 484 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (121 mg, 316 μmol) and 3-(3-methylphenyl)-5-(4,4,5,5-tetramethyl-1,3,2-dioxaborolan-2-yl)pyridin-2-amine (147 mg, 474 μmol).

LC-MS (Method 1): Rt=1.00 min; MS (ESIpos): m/z=417 [M+H]⁺

¹H-NMR (400 MHz, DMSO-d6) δ [ppm]: 0.852 (0.44), 0.996 (5.69), 1.014 (12.60), 1.032 (5.77), 1.232 (1.46), 2.028 (1.15), 2.042 (1.69), 2.055 (2.22), 2.073 (0.97), 2.084 (0.45), 2.331 (2.22), 2.352 (2.76), 2.369 (16.00), 2.522 (9.23), 2.531 (3.83), 2.539 (5.86), 2.673 (2.17), 3.013 (0.79), 3.031 (2.40), 3.045 (2.72), 3.048 (2.64), 3.063 (2.39), 3.081 (0.70), 3.369 (2.30), 3.396 (8.46), 3.467 (0.70), 3.485 (1.15), 3.499 (0.87), 3.507 (0.76), 3.524 (0.43), 4.146 (2.25), 4.163 (3.85), 4.181 (2.14), 5.523 (0.57), 5.668 (5.62), 6.131 (1.01), 6.145 (2.04), 6.159 (1.00), 6.392 (8.45), 7.186 (1.98), 7.205 (2.25), 7.242 (0.59), 7.256 (1.55), 7.276 (2.47), 7.288 (3.70), 7.306 (0.45), 7.311 (0.48), 7.326 (0.48), 7.343 (2.62), 7.362 (3.43), 7.381 (1.32), 7.636 (4.42), 7.642 (4.56), 8.347 (4.76), 8.353 (4.63).

### Example 485

### (rac)-2′-(6-amino-5-methoxypyridin-3-yl)-N-ethyl-5′,6′-dihydrospiro[pyrrolidine-3,4′-pyrrolo[1,2-b]pyrazole]-1-carboxamide

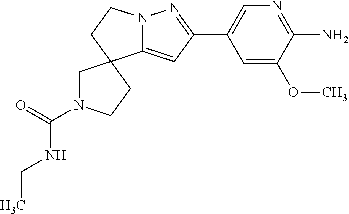 [SMILES: CCNC(=O)N1CCC2(CCn3nc(-c4cnc(N)c(OC)c4)cc32)C1]

Example 485 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (200 mg, 523 μmol) and 3-methoxy-5-(4,4,5,5-tetramethyl-1,3,2-dioxaborolan-2-yl)pyridin-2-amine (196 mg, 785 μmol).

LC-MS (Method 1): Rt=0.74 min; MS (ESIpos): m/z=357 [M+H]⁺

¹H-NMR (400 MHz, DMSO-d6) δ [ppm]: 1.002 (4.43), 1.019 (10.22), 1.037 (4.51), 1.066 (0.55), 2.028 (0.75), 2.042 (1.18), 2.057 (1.61), 2.075 (0.67), 2.332 (0.61), 2.523 (2.55), 2.532 (1.91), 2.669 (0.86), 2.673 (0.61), 3.019 (0.57), 3.036 (1.78), 3.050 (1.95), 3.054 (1.89), 3.068 (1.72), 3.086 (0.50), 3.360 (0.53), 3.373 (0.49), 3.378 (0.93), 3.386 (0.86), 3.399 (5.67), 3.422 (0.54), 3.467 (0.46), 3.485 (0.79), 3.492 (0.48), 3.499 (0.58), 3.507 (0.46), 3.511 (0.46), 3.566 (0.65), 3.817 (16.00), 4.144 (1.69), 4.162 (2.76), 4.179 (1.63), 5.755 (4.26), 5.758 (3.59), 6.138 (0.69), 6.152 (1.36), 6.166 (0.67), 6.349 (7.02), 7.315 (2.75), 7.319 (2.77), 7.910 (3.67), 7.915 (3.55).

### Example 486

### (rac)-2′-[6-amino-5-(3-fluorophenyl)pyridin-3-yl]-N-ethyl-5′,6′-dihydrospiro[pyrrolidine-3,4′-pyrrolo[1,2-b]pyrazole]-1-carboxamide

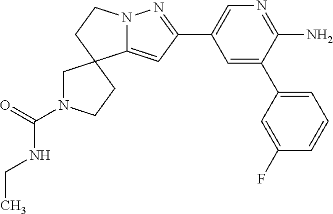 [SMILES: CCNC(=O)N1CCC2(CCn3nc(-c4cnc(N)c(-c5cccc(F)c5)c4)cc32)C1]

Example 486 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (75.0 mg, 196 μmol) and [6-amino-5-(3-fluorophenyl)pyridin-3-yl]boronic acid (508 mg, 2.19 mmol).

¹H NMR (400 MHz, DMSO-d₆) δ ppm 1.01 (t, 3H) 2.00-2.10 (m, 2H) 2.52-2.55 (m, 2H) 3.01-3.09 (m, 2H) 3.36-3.52 (m, 4H) 4.16 (t, 2H) 5.81 (s, 2H) 6.14 (t, 1H) 6.41 (s, 1H) 7.21 (t, 1H) 7.29-7.32 (m, 1H) 7.33 (s, 1H) 7.51 (td, 1H) 7.68 (d, 1H) 8.38 (d, 1H)

### Example 487

### (rac)-2′-(2-amino[3,3′-bipyridin]-5-yl)-N-ethyl-5′,6′-dihydrospiro[pyrrolidine-3,4′-pyrrolo[1,2-b]pyrazole]-1-carboxamide

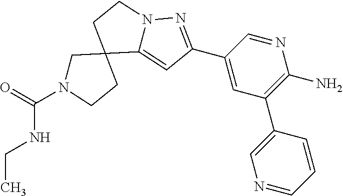 [SMILES: CCNC(=O)N1CCC2(CCn3nc(-c4cnc(N)c(-c5cccnc5)c4)cc32)C1]

Example 487 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (75.0 mg, 196 μmol) and 5-(4,4,5,5-tetramethyl-1,3,2-dioxaborolan-2-yl)[3,3′-bipyridin]-2-amine (87.4 mg, 294 μmol).

¹H NMR (400 MHz, DMSO-d₆) δ ppm 0.99-1.07 (m, 3H) 2.00-2.12 (m, 2H) 2.51-2.54 (m, 2H) 3.01-3.09 (m, 2H) 3.35-3.52 (m, 4H) 4.17 (t, 2H) 5.87 (s, 2H) 6.14 (t, 1H) 6.42 (s, 1H) 7.48 (ddd, 1H) 7.69 (d, 1H) 7.90 (dt, 1H) 8.40 (d, 1H) 8.58 (dd, 1H) 8.64-8.68 (m, 1H)

### Example 488

### (rac)-2′-[6-amino-5-(2-fluorophenyl)pyridin-3-yl]-N-ethyl-5′,6′-dihydrospiro[pyrrolidine-3,4′-pyrrolo[1,2-b]pyrazole]-1-carboxamide

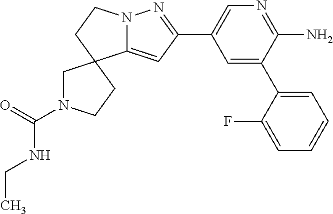 [SMILES: CCNC(=O)N1CCC2(CCn3nc(-c4cnc(N)c(-c5ccccc5F)c4)cc32)C1]

Example 488 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (75.0 mg, 196 μmol) and [6-amino-5-(2-fluorophenyl)pyridin-3-yl]boronic acid (195 mg, 840 μmol).

LC-MS (Method 1): Rt=0.92 min; MS (ESIpos): m/z=421 [M+H]⁺

¹H-NMR (400 MHz, DMSO-d6) δ [ppm]: 0.994 (6.69), 1.012 (16.00), 1.022 (0.73), 1.029 (7.08), 2.024 (1.24), 2.030 (1.25), 2.041 (1.75), 2.048 (2.41), 2.066 (1.08), 2.332 (0.69), 2.518 (4.52), 2.523 (3.10), 2.528 (2.80), 3.011 (0.82), 3.029 (2.59), 3.043 (2.94), 3.046 (2.77), 3.060 (2.58), 3.078 (0.75), 3.366 (1.73), 3.373 (1.16), 3.391 (8.39), 3.409 (0.84), 3.419 (0.69), 3.461 (0.66), 3.479 (1.20), 3.486 (0.73), 3.494 (0.85), 3.504 (0.76), 3.519 (0.44), 4.140 (2.44), 4.158 (4.06), 4.175 (2.35), 5.675 (6.04), 6.126 (1.09), 6.140 (2.19), 6.153 (1.09), 6.382 (10.76), 7.281 (1.24), 7.283 (1.93), 7.286 (1.50), 7.289 (1.44), 7.300 (2.70), 7.302 (3.19), 7.307 (2.08), 7.310 (2.24), 7.314 (1.61), 7.318 (2.36), 7.321 (2.07), 7.333 (1.64), 7.395 (1.21), 7.399 (1.69), 7.414 (2.10), 7.419 (2.34), 7.431 (1.32), 7.433 (1.45), 7.436 (1.34), 7.444 (1.16), 7.449 (1.50), 7.452 (1.18), 7.456 (1.02), 7.463 (0.95), 7.465 (1.12), 7.469 (1.28), 7.475 (0.59), 7.483 (0.60), 7.488 (0.52), 7.634 (3.90), 7.640 (3.93), 8.397 (5.77), 8.403 (5.66).

### Example 489

### 2′-{6-amino-5-[(1R)-1-(2,6-dichloro-3-fluorophenyl)ethoxy]pyridin-3-yl}-N-ethyl-5′,6′-dihydrospiro[pyrrolidine-3,4′-pyrrolo[1,2-b]pyrazole]-1-carboxamide (Diastereomeric Mixture)

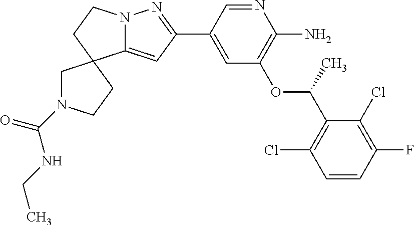 [SMILES: CCNC(=O)N1CCC2(CCn3nc(-c4cnc(N)c(O[C@H](C)c5c(Cl)ccc(F)c5Cl)c4)cc32)C1]

Example 489 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (75.0 mg, 196 μmol) and 3-[(1R)-1-(2,6-dichloro-3-fluorophenyl)ethoxy]-5-(4,4,5,5-tetramethyl-1,3,2-dioxaborolan-2-yl)pyridin-2-amine (126 mg, 294 μmol).

LC-MS (Method 1): Rt=1.10 min; MS (ESIpos): m/z=533 [M+H]⁺

¹H-NMR (400 MHz, DMSO-d6) δ [ppm]: 1.002 (0.64), 1.005 (0.67), 1.020 (1.39), 1.023 (1.43), 1.038 (0.67), 1.041 (0.67), 1.776 (1.58), 1.792 (1.59), 2.074 (0.61), 2.468 (0.47), 2.518 (0.73), 2.522 (0.50), 2.539 (16.00), 3.058 (0.41), 3.374 (1.41), 4.126 (0.66), 5.786 (0.96), 6.131 (1.20), 7.079 (0.65), 7.878 (0.88), 7.883 (0.87).

### Example 490

### (rac)-2-[6-amino-5-(2-fluorophenyl)pyridin-3-yl]-N-ethyl-6,7-dihydro-5H-spiro[pyrazolo[1,5-a]pyridine-4,3′-pyrrolidine]-1′-carboxamide

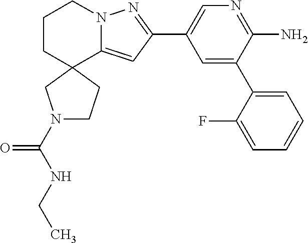 [SMILES: CCNC(=O)N1CCC2(CCCn3nc(-c4cnc(N)c(-c5ccccc5F)c4)cc32)C1]

Example 490 was prepared in analogy to Example 461 using (rac)-1′-(ethylcarbamoyl)-6,7-dihydro-5H-spiro[pyrazolo[1,5-a]pyridine-4,3′-pyrrolidin]-2-yl trifluoromethanesulfonate (100 mg, 252 μmol) and [6-amino-5-(2-fluorophenyl)pyridin-3-yl]boronic acid (197 mg, 849 μmol).

LC-MS (Method 1): Rt=0.95 min; MS (ESIpos): m/z=435 [M+H]⁺

¹H-NMR (400 MHz, DMSO-d6) δ [ppm]: 0.986 (7.18), 1.004 (16.00), 1.022 (7.74), 1.755 (2.43), 1.763 (2.59), 1.772 (2.68), 1.806 (0.52), 1.938 (0.77), 1.956 (1.27), 1.969 (2.00), 1.981 (2.22), 1.988 (2.21), 1.998 (2.06), 2.024 (1.45), 2.086 (0.84), 2.106 (1.60), 2.125 (1.04), 2.136 (1.20), 2.155 (0.62), 2.522 (5.98), 3.007 (1.00), 3.025 (3.22), 3.039 (3.87), 3.057 (3.29), 3.075 (1.00), 3.284 (0.43), 3.351 (2.97), 3.370 (10.34), 3.396 (1.60), 3.452 (0.84), 3.464 (1.05), 3.472 (1.29), 3.485 (1.18), 3.497 (0.76), 3.510 (0.54), 4.048 (2.57), 4.063 (4.99), 4.078 (2.36), 5.529 (0.79), 5.682 (7.55), 6.114 (1.43), 6.127 (2.76), 6.141 (1.42), 6.257 (0.49), 6.463 (9.25), 6.620 (0.46), 6.632 (0.51), 6.637 (0.51), 6.650 (0.50), 7.283 (2.65), 7.300 (4.29), 7.309 (3.09), 7.317 (3.11), 7.332 (2.04), 7.354 (0.41), 7.373 (0.59), 7.391 (1.68), 7.395 (1.85), 7.410 (2.52), 7.414 (2.78), 7.431 (2.10), 7.445 (1.42), 7.449 (1.90), 7.465 (1.43), 7.469 (1.48), 7.483 (0.69), 7.488 (0.57), 7.616 (4.78), 7.621 (4.80), 7.979 (0.48), 7.983 (0.48), 7.991 (0.47), 7.996 (0.42), 8.376 (5.87), 8.381 (5.71), 8.545 (1.49).

### Example 491

### (rac)-2′-[6-amino-5-(2-methoxyphenyl)pyridin-3-yl]-N-ethyl-5′,6′-dihydrospiro[pyrrolidine-3,4′-pyrrolo[1,2-b]pyrazole]-1-carboxamide

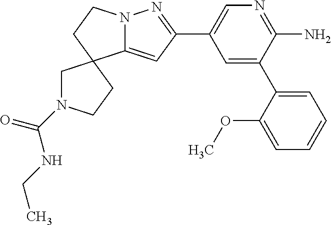 [SMILES: CCNC(=O)N1CCC2(CCn3nc(-c4cnc(N)c(-c5ccccc5OC)c4)cc32)C1]

Example 491 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (75.0 mg, 196 μmol) and [6-amino-5-(2-methoxyphenyl)pyridin-3-yl]boronic acid (337 mg, 1.38 mmol).

1H NMR (400 MHz, DMSO-d6) δ ppm 1.01 (t, 3H) 1.18-1.36 (m, 1H) 1.99-2.12 (m, 2H) 2.52-2.55 (m, 1H) 3.00-3.09 (m, 2H) 3.35-3.52 (m, 4H) 3.76 (s, 3H) 4.15 (t, 2H) 5.36 (s, 2H) 6.14 (t, 1H) 6.36 (s, 1H) 7.04 (td, 1H) 7.12 (d, 1H) 7.20 (dd, 1H) 7.40 (t, 1H) 7.56 (d, 1H) 8.34 (d, 1H)

### Example 492

### (rac)-2′-(2-amino[3,4′-bipyridin]-5-yl)-N-ethyl-5′,6′-dihydrospiro[pyrrolidine-3,4′-pyrrolo[1,2-b]pyrazole]-1-carboxamide

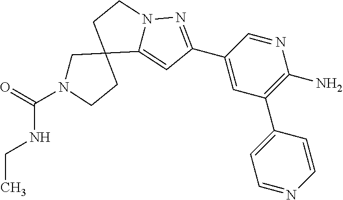 [SMILES: CCNC(=O)N1CCC2(CCn3nc(-c4cnc(N)c(-c5ccncc5)c4)cc32)C1]

Example 492 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (75.0 mg, 196 μmol) and 5-(4,4,5,5-tetramethyl-1,3,2-dioxaborolan-2-yl)[3,4′-bipyridin]-2-amine (87.4 mg, 294 μmol).

LC-MS (Method 1): Rt=0.73 min; MS (ESIpos): m/z=404 [M+H]⁺

¹H-NMR (400 MHz, DMSO-d6) δ [ppm]: 0.997 (6.76), 1.015 (16.00), 1.024 (1.17), 1.033 (7.26), 1.042 (1.17), 1.060 (0.48), 1.156 (0.54), 1.174 (1.10), 1.192 (0.55), 1.232 (0.42), 2.033 (1.52), 2.051 (2.80), 2.063 (1.31), 2.069 (1.26), 2.084 (1.02), 2.518 (6.35), 2.522 (5.95), 2.539 (10.09), 3.014 (0.86), 3.032 (2.71), 3.046 (3.09), 3.049 (2.92), 3.064 (2.71), 3.081 (0.88), 3.109 (0.47), 3.370 (2.60), 3.379 (2.53), 3.387 (2.38), 3.396 (7.43), 3.399 (7.10), 3.424 (1.35), 3.441 (0.48), 3.463 (0.86), 3.480 (1.48), 3.488 (0.84), 3.495 (0.99), 3.505 (1.02), 3.521 (0.51), 4.152 (2.48), 4.170 (4.03), 4.187 (2.39), 6.057 (2.99), 6.134 (1.17), 6.148 (2.26), 6.162 (1.14), 6.430 (10.09), 7.536 (6.49), 7.540 (4.15), 7.547 (4.17), 7.552 (6.58), 7.580 (1.12), 7.584 (0.70), 7.591 (0.70), 7.595 (1.11), 7.758 (4.64), 7.763 (4.79), 7.772 (0.74), 7.778 (0.66), 8.345 (0.86), 8.352 (0.80), 8.418 (5.98), 8.424 (5.77), 8.639 (7.26), 8.642 (4.64), 8.649 (4.34), 8.653 (6.90).

### Example 493

### (rac)-2′-{6-amino-5-[3-(methanesulfonyl)phenyl]pyridin-3-yl}-N-ethyl-5′,6′-dihydrospiro[pyrrolidine-3,4′-pyrrolo[1,2-b]pyrazole]-1-carboxamide

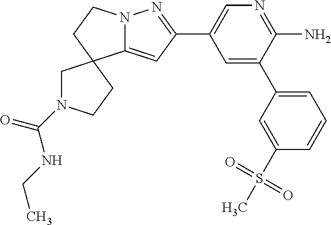 [SMILES: CCNC(=O)N1CCC2(CCn3nc(-c4cnc(N)c(-c5cccc(S(C)(=O)=O)c5)c4)cc32)C1]

Example 493 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (75.0 mg, 196 μmol) and 3-[3-(methanesulfonyl)phenyl]-5-(4,4,5,5-tetramethyl-1,3,2-dioxaborolan-2-yl)pyridin-2-amine (472 mg, 1.26 mmol).

LC-MS (Method 1): Rt=0.80 min; MS (ESIpos): m/z=481 [M+H]⁺

¹H-NMR (500 MHz, DMSO-d6) δ [ppm]: 1.006 (4.29), 1.020 (8.95), 1.034 (4.42), 2.038 (0.85), 2.050 (1.57), 2.064 (1.86), 2.079 (0.74), 2.089 (0.41), 2.525 (3.42), 2.543 (12.61), 3.026 (0.60), 3.040 (1.87), 3.052 (2.16), 3.054 (2.14), 3.066 (1.83), 3.080 (0.58), 3.281 (16.00), 3.369 (0.42), 3.383 (1.28), 3.388 (0.85), 3.404 (5.40), 3.417 (0.80), 3.428 (0.46), 3.475 (0.50), 3.491 (0.83), 3.496 (0.60), 3.502 (0.66), 3.507 (0.58), 4.159 (1.77), 4.174 (3.00), 4.187 (1.73), 5.916 (4.49), 6.129 (0.82), 6.141 (1.61), 6.152 (0.81), 6.408 (5.88), 7.730 (3.84), 7.734 (3.28), 7.744 (2.76), 7.760 (1.74), 7.840 (1.77), 7.856 (1.33), 7.913 (1.63), 7.929 (1.40), 7.989 (1.94), 7.992 (3.09), 8.413 (3.33), 8.417 (3.30).

### Example 494

### (rac)-2′-[6-amino-5-(phenylsulfanyl)pyridin-3-yl]-N-ethyl-5′,6′-dihydrospiro[pyrrolidine-3,4′-pyrrolo[1,2-b]pyrazole]-1-carboxamide

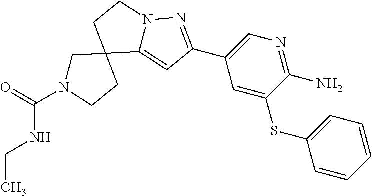 [SMILES: CCNC(=O)N1CCC2(CCn3nc(-c4cnc(N)c(Sc5ccccc5)c4)cc32)C1]

Example 494 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (600 mg, 1.57 mmol) and [6-amino-5-(phenylsulfanyl)pyridin-3-yl]boronic acid (1.65 g, 6.72 mmol).

LC-MS (Method 1): Rt=1.03 min; MS (ESIpos): m/z=435 [M+H]⁺

¹H-NMR (400 MHz, DMSO-d6) δ [ppm]: 0.995 (5.69), 1.004 (2.25), 1.013 (13.09), 1.031 (5.95), 2.044 (1.98), 2.518 (16.00), 2.523 (11.50), 3.030 (2.51), 3.044 (2.64), 3.062 (2.12), 3.365 (2.38), 3.390 (7.54), 3.478 (1.06), 4.136 (2.25), 4.153 (3.57), 4.170 (1.98), 5.194 (0.93), 5.758 (9.92), 6.145 (1.98), 6.158 (0.93), 6.264 (5.16), 6.397 (9.26), 7.158 (3.83), 7.176 (5.16), 7.179 (4.36), 7.203 (2.64), 7.222 (1.85), 7.295 (3.83), 7.314 (4.89), 7.332 (2.25), 7.969 (4.50), 7.974 (4.50), 8.440 (4.89), 8.446 (4.63).

### Example 495

### (rac)-2′-{6-amino-5-[4-(methanesulfonyl)phenyl]pyridin-3-yl}-N-ethyl-5′,6′-dihydrospiro[pyrrolidine-3,4′-pyrrolo[1,2-b]pyrazole]-1-carboxamide

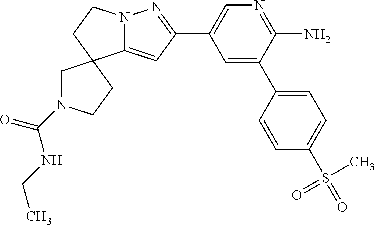 [SMILES: CCNC(=O)N1CCC2(CCn3nc(-c4cnc(N)c(-c5ccc(S(C)(=O)=O)cc5)c4)cc32)C1]

Example 495 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (150 mg, 392 μmol) and {6-amino-5-[4-(methanesulfonyl)phenyl]pyridin-3-yl}boronic acid (491 mg, 1.68 mmol).

LC-MS (Method 1): Rt=0.78 min; MS (ESIpos): m/z=481 [M+H]⁺

¹H-NMR (500 MHz, DMSO-d6) δ [ppm]: 1.005 (3.55), 1.013 (0.77), 1.019 (8.14), 1.027 (0.44), 1.034 (3.58), 1.069 (1.85), 2.042 (0.79), 2.056 (1.70), 2.070 (0.89), 2.518 (1.56), 2.522 (1.50), 2.525 (1.21), 2.536 (1.26), 2.543 (16.00), 2.550 (1.78), 2.564 (1.21), 2.642 (0.40), 3.040 (0.98), 3.051 (1.14), 3.065 (0.93), 3.170 (1.01), 3.381 (0.67), 3.400 (2.21), 3.411 (2.26), 3.422 (0.45), 3.429 (1.05), 3.442 (0.43), 3.458 (0.44), 3.471 (0.80), 3.477 (0.41), 3.484 (0.50), 3.491 (0.52), 4.184 (1.16), 4.198 (1.80), 4.212 (1.14), 6.142 (0.41), 6.153 (0.72), 6.553 (3.19), 7.792 (0.45), 7.796 (3.26), 7.800 (1.05), 7.809 (1.13), 7.813 (3.61), 7.817 (0.53), 8.063 (3.56), 8.067 (1.09), 8.076 (1.05), 8.080 (2.93), 8.096 (0.84), 8.313 (0.80), 8.401 (2.41), 8.405 (2.38).

### Example 496

### (rac)-2′-[5-(4-acetylphenyl)-6-aminopyridin-3-yl]-N-ethyl-5′,6′-dihydrospiro[pyrrolidine-3,4′-pyrrolo[1,2-b]pyrazole]-1-carboxamide

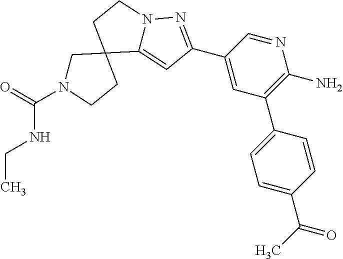 [SMILES: CCNC(=O)N1CCC2(CCn3nc(-c4cnc(N)c(-c5ccc(C(C)=O)cc5)c4)cc32)C1]

Example 496 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (200 mg, 523 μmol) and [5-(4-acetylphenyl)-6-aminopyridin-3-yl]boronic acid (574 mg, 2.24 mmol).

LC-MS (Method 1): Rt=0.88 min; MS (ESIpos): m/z=445 [M+H]⁺

¹H-NMR (400 MHz, DMSO-d6) δ [ppm]: 0.996 (1.01), 1.014 (2.20), 1.032 (1.01), 1.066 (0.49), 1.156 (1.43), 2.055 (0.38), 2.074 (0.35), 2.518 (1.72), 2.522 (1.04), 2.534 (0.50), 2.539 (0.41), 2.609 (16.00), 2.620 (4.67), 3.031 (0.40), 3.046 (0.48), 3.049 (0.45), 3.064 (0.40), 3.399 (1.39), 4.150 (0.37), 4.168 (0.61), 4.185 (0.35), 5.709 (2.12), 5.844 (0.88), 6.145 (0.35), 6.409 (1.48), 6.542 (0.46), 6.666 (1.38), 6.679 (1.47), 6.684 (1.43), 6.697 (1.48), 7.367 (1.30), 7.372 (1.34), 7.386 (1.27), 7.390 (1.21), 7.594 (3.23), 7.598 (1.13), 7.610 (1.27), 7.615 (3.41), 7.645 (0.97), 7.666 (1.03), 7.705 (0.75), 7.711 (0.77), 7.979 (1.35), 7.984 (1.42), 7.991 (1.41), 7.996 (1.30), 8.021 (3.60), 8.026 (1.20), 8.038 (2.19), 8.042 (3.38), 8.059 (0.93), 8.396 (0.82), 8.402 (0.80).

### Example 497

### (rac)-2′-{6-amino-5-[4-(methanesulfinyl)phenyl]pyridin-3-yl}-N-ethyl-5′,6′-dihydrospiro[pyrrolidine-3,4′-pyrrolo[1,2-b]pyrazole]-1-carboxamide

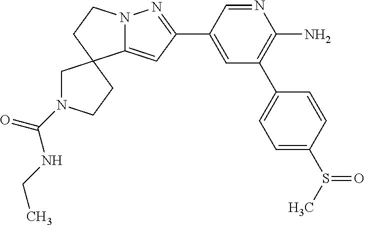 [SMILES: CCNC(=O)N1CCC2(CCn3nc(-c4cnc(N)c(-c5ccc(S(C)=O)cc5)c4)cc32)C1]

Example 497 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (200 mg, 523 μmol) and {6-amino-5-[4-(methanesulfinyl)phenyl]pyridin-3-yl}boronic acid (619 mg, 2.24 mmol).

LC-MS (Method 1): Rt=0.74 min; MS (ESIpos): m/z=465 [M+H]⁺

¹H-NMR (400 MHz, DMSO-d6) δ [ppm]: 0.996 (3.64), 1.014 (8.65), 1.032 (3.85), 2.034 (0.73), 2.052 (1.31), 2.071 (0.58), 2.074 (0.65), 2.518 (4.44), 2.523 (2.44), 2.535 (1.45), 2.539 (0.80), 2.787 (1.64), 2.793 (16.00), 3.013 (0.44), 3.031 (1.38), 3.045 (1.56), 3.048 (1.53), 3.063 (1.38), 3.373 (0.98), 3.382 (0.84), 3.398 (4.11), 3.417 (0.40), 3.482 (0.65), 3.489 (0.40), 3.497 (0.47), 3.508 (0.44), 4.149 (1.31), 4.166 (2.07), 4.183 (1.20), 5.821 (2.91), 6.131 (0.58), 6.145 (1.20), 6.159 (0.62), 6.408 (5.67), 7.682 (2.80), 7.686 (1.16), 7.695 (2.98), 7.703 (5.13), 7.762 (5.05), 7.767 (1.38), 7.778 (1.09), 7.784 (2.84), 8.390 (3.09), 8.396 (2.98).

### Example 498

### (rac)-2′-[6-amino-5-(3-methoxyphenyl)pyridin-3-yl]-N-ethyl-5′,6′-dihydrospiro[pyrrolidine-3,4′-pyrrolo[1,2-b]pyrazole]-1-carboxamide

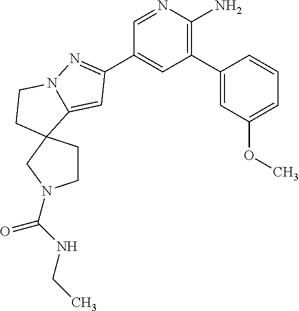 [SMILES: CCNC(=O)N1CCC2(CCn3nc(-c4cnc(N)c(-c5cccc(OC)c5)c4)cc32)C1]

Example 498 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (170 mg, 445 μmol) and [6-amino-5-(3-methoxyphenyl)pyridin-3-yl]boronic acid (163 mg, 667 μmol).

LC-MS (Method 1): Rt=0.90 min; MS (ESIpos): m/z=433 [M+H]⁺

¹H-NMR (400 MHz, DMSO-d6) δ [ppm]: 0.995 (3.30), 1.013 (7.93), 1.031 (3.49), 1.064 (0.85), 2.028 (0.57), 2.041 (0.80), 2.054 (1.13), 2.071 (0.47), 2.518 (2.45), 2.522 (1.46), 2.530 (1.23), 3.031 (1.23), 3.044 (1.42), 3.048 (1.32), 3.062 (1.23), 3.368 (1.32), 3.375 (0.80), 3.395 (4.06), 3.412 (0.47), 3.485 (0.57), 3.499 (0.42), 3.803 (16.00), 4.146 (1.13), 4.163 (1.94), 4.181 (1.09), 5.700 (2.78), 6.133 (0.52), 6.147 (1.04), 6.161 (0.52), 6.396 (5.10), 6.934 (0.66), 6.937 (0.80), 6.941 (0.80), 6.943 (0.90), 6.955 (0.76), 6.957 (0.80), 6.961 (0.99), 6.964 (0.94), 7.006 (1.13), 7.010 (1.70), 7.016 (1.37), 7.020 (1.09), 7.023 (1.23), 7.039 (0.99), 7.041 (1.13), 7.045 (0.76), 7.370 (1.27), 7.389 (1.70), 7.409 (0.99), 7.654 (2.55), 7.660 (2.60), 8.353 (3.07), 8.359 (3.02).

### Example 499

### 2′-{6-amino-5-[(1R)-1-(2,6-dichloro-3-fluorophenyl)ethoxy]pyridin-3-yl}-N-ethyl-5′,6′-dihydrospiro[azetidine-3,4′-pyrrolo[1,2-b]pyrazole]-1-carboxamide

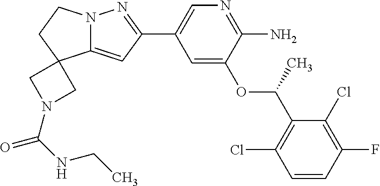 [SMILES: CCNC(=O)N1CC2(CCn3nc(-c4cnc(N)c(O[C@H](C)c5c(Cl)ccc(F)c5Cl)c4)cc32)C1]

Example 499 was prepared in analogy to Example 461 using 1-(ethylcarbamoyl)-5′,6′-dihydrospiro[azetidine-3,4′-pyrrolo[1,2-b]pyrazol]-2′-yl trifluoromethanesulfonate (75.0 mg, 204 μmol) and 3-[(1R)-1-(2,6-dichloro-3-fluorophenyl)ethoxy]-5-(4,4,5,5-tetramethyl-1,3,2-dioxaborolan-2-yl)pyridin-2-amine (104 mg, 244 μmol).

LC-MS (Method 1): Rt=1.10 min; MS (ESIpos): m/z=519 [M+H]⁺

¹H-NMR (400 MHz, DMSO-d6) δ [ppm]: 0.992 (7.03), 1.009 (15.84), 1.027 (7.26), 1.052 (0.70), 1.065 (16.00), 1.154 (0.92), 1.172 (1.67), 1.189 (0.92), 1.231 (2.19), 1.774 (10.41), 1.791 (10.31), 1.810 (1.52), 1.987 (3.00), 2.331 (0.72), 2.518 (4.09), 2.522 (2.56), 2.539 (1.78), 2.673 (0.72), 2.782 (2.43), 2.800 (4.11), 2.817 (2.63), 2.994 (0.97), 3.012 (3.03), 3.026 (3.35), 3.030 (3.31), 3.044 (2.96), 3.062 (0.90), 3.165 (4.09), 3.920 (1.00), 3.927 (1.07), 3.947 (10.63), 3.964 (1.27), 3.970 (1.00), 4.016 (0.78), 4.034 (0.78), 4.052 (2.82), 4.070 (4.18), 4.087 (2.57), 5.758 (1.78), 5.784 (5.97), 6.036 (0.65), 6.053 (2.47), 6.070 (2.47), 6.086 (0.67), 6.413 (11.47), 6.433 (1.41), 6.447 (2.89), 6.461 (1.39), 7.136 (4.30), 7.140 (4.32), 7.413 (2.06), 7.435 (4.26), 7.457 (2.94), 7.534 (2.33), 7.547 (2.43), 7.557 (1.81), 7.569 (1.69), 7.908 (5.66), 7.913 (5.62).

### Example 500

### (rac)-2′-{6-amino-5-[4-(cyclopropanesulfonyl)phenyl]pyridin-3-yl}-N-ethyl-5′,6′-dihydrospiro[pyrrolidine-3,4′-pyrrolo[1,2-b]pyrazole]-1-carboxamide

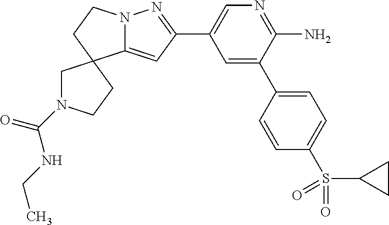 [SMILES: CCNC(=O)N1CCC2(CCn3nc(-c4cnc(N)c(-c5ccc(S(=O)(=O)C6CC6)cc5)c4)cc32)C1]

Example 500 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (100 mg, 262 μmol) and {6-amino-5-[4-(cyclopropanesulfonyl)phenyl]pyridin-3-yl}boronic acid (125 mg, 392 μmol).

LC-MS (Method 1): Rt=0.88 min; MS (ESIpos): m/z=507 [M+H]⁺

¹H-NMR (400 MHz, DMSO-d6) δ [ppm]: 0.996 (6.70), 1.014 (16.00), 1.032 (7.19), 1.044 (0.65), 1.060 (2.71), 1.066 (3.99), 1.074 (1.98), 1.080 (2.58), 1.085 (2.60), 1.097 (1.09), 1.105 (0.41), 1.112 (0.45), 1.118 (0.42), 1.131 (0.47), 1.143 (1.41), 1.152 (3.46), 1.156 (3.15), 1.163 (3.42), 1.169 (2.56), 1.182 (0.68), 2.031 (1.41), 2.050 (2.60), 2.062 (1.18), 2.068 (1.15), 2.518 (6.02), 2.537 (2.30), 2.860 (0.60), 2.872 (1.25), 2.879 (1.22), 2.884 (0.88), 2.892 (2.22), 2.898 (0.80), 2.903 (1.18), 2.911 (1.09), 2.923 (0.54), 3.013 (0.78), 3.031 (2.48), 3.045 (2.79), 3.048 (2.66), 3.063 (2.45), 3.081 (0.70), 3.362 (0.76), 3.370 (0.80), 3.377 (1.41), 3.395 (6.22), 3.398 (5.96), 3.423 (0.91), 3.463 (0.65), 3.480 (1.25), 3.488 (0.71), 3.495 (0.83), 3.505 (0.83), 3.520 (0.41), 4.150 (2.19), 4.168 (3.60), 4.185 (2.13), 5.794 (0.81), 5.933 (5.21), 6.130 (1.05), 6.143 (2.19), 6.157 (1.07), 6.420 (10.56), 6.671 (0.58), 6.683 (0.65), 6.689 (0.60), 6.701 (0.65), 7.390 (0.49), 7.395 (0.52), 7.409 (0.47), 7.414 (0.49), 7.708 (1.25), 7.713 (0.42), 7.726 (5.23), 7.732 (5.27), 7.763 (6.07), 7.767 (2.06), 7.779 (2.22), 7.784 (7.30), 7.932 (1.53), 7.938 (0.60), 7.948 (8.08), 7.953 (2.95), 7.964 (2.08), 7.969 (6.04), 7.991 (0.65), 7.996 (0.62), 8.003 (0.62), 8.008 (0.57), 8.414 (5.94), 8.420 (5.97).

### Example 501

### (rac)-2′-[6-amino-5-(4-sulfamoylphenyl)pyridin-3-yl]-N-ethyl-5′,6′-dihydrospiro[pyrrolidine-3,4′-pyrrolo[1,2-b]pyrazole]-1-carboxamide

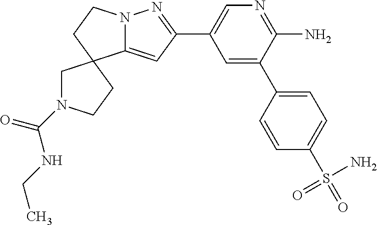 [SMILES: CCNC(=O)N1CCC2(CCn3nc(-c4cnc(N)c(-c5ccc(S(N)(=O)=O)cc5)c4)cc32)C1]

Example 501 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (100 mg, 262 μmol) and [6-amino-5-(4-sulfamoylphenyl)pyridin-3-yl]boronic acid (115 mg, 392 μmol).

LC-MS (Method 1): Rt=0.68 min; MS (ESIpos): m/z=482 [M+H]⁺

¹H-NMR (400 MHz, DMSO-d6) δ [ppm]: 0.997 (6.85), 1.015 (16.00), 1.033 (7.21), 2.034 (1.33), 2.053 (2.46), 2.062 (1.21), 2.071 (1.14), 2.074 (1.05), 2.083 (0.49), 2.518 (5.50), 2.535 (2.60), 3.014 (0.81), 3.032 (2.63), 3.046 (2.93), 3.050 (2.86), 3.064 (2.60), 3.081 (0.77), 3.356 (0.90), 3.375 (1.46), 3.382 (1.21), 3.400 (7.20), 3.418 (0.83), 3.425 (0.70), 3.466 (0.68), 3.483 (1.25), 3.490 (0.77), 3.497 (0.87), 3.508 (0.81), 3.523 (0.45), 4.150 (2.22), 4.168 (3.81), 4.185 (2.19), 5.813 (0.69), 5.859 (5.22), 6.131 (1.11), 6.145 (2.27), 6.159 (1.11), 6.411 (9.68), 7.411 (2.20), 7.679 (6.19), 7.683 (2.30), 7.697 (6.87), 7.700 (8.92), 7.720 (0.81), 7.742 (0.95), 7.879 (1.47), 7.884 (7.76), 7.889 (2.68), 7.901 (2.24), 7.905 (6.09), 8.301 (0.63), 8.307 (0.62), 8.397 (5.57), 8.402 (5.62).

### Example 502

### (rac)-2′-{6-amino-5-[4-(methylsulfamoyl)phenyl]pyridin-3-yl}-N-ethyl-5′,6′-dihydrospiro[pyrrolidine-3,4′-pyrrolo[1,2-b]pyrazole]-1-carboxamide

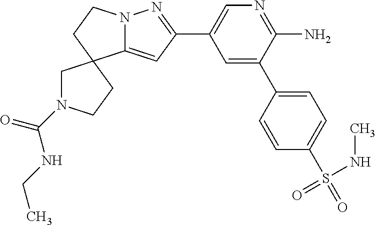 [SMILES: CCNC(=O)N1CCC2(CCn3nc(-c4cnc(N)c(-c5ccc(S(=O)(=O)NC)cc5)c4)cc32)C1]

Example 502 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (100 mg, 262 μmol) and {6-amino-5-[4-(methylsulfamoyl)phenyl]pyridin-3-yl}boronic acid (120 mg, 392 μmol).

LC-MS (Method 1): Rt=0.78 min; MS (ESIpos): m/z=496 [M+H]⁺

¹H-NMR (400 MHz, DMSO-d6) δ [ppm]: 0.996 (6.89), 1.014 (16.00), 1.032 (7.45), 1.137 (0.44), 1.232 (0.84), 1.256 (0.88), 1.296 (0.42), 2.031 (1.65), 2.050 (3.06), 2.061 (1.58), 2.067 (1.51), 2.458 (7.49), 2.468 (8.16), 2.536 (5.59), 2.539 (8.32), 3.013 (0.98), 3.031 (2.88), 3.045 (3.32), 3.049 (3.31), 3.063 (2.99), 3.081 (1.11), 3.359 (3.31), 3.369 (3.64), 3.376 (4.10), 3.398 (9.55), 3.419 (3.01), 3.462 (1.83), 3.480 (2.34), 3.488 (1.72), 3.495 (1.79), 3.506 (1.72), 3.521 (1.09), 4.148 (2.46), 4.166 (4.25), 4.183 (2.58), 5.901 (5.73), 6.133 (1.20), 6.146 (2.46), 6.159 (1.30), 6.414 (10.65), 7.524 (1.21), 7.535 (1.27), 7.711 (7.53), 7.717 (6.05), 7.730 (8.28), 7.830 (8.44), 7.852 (5.89), 8.403 (4.20), 8.408 (4.31).

### Example 503

### (rac)-2′-{6-amino-5-[4-(dimethylsulfamoyl)phenyl]pyridin-3-yl}-N-ethyl-5′,6′-dihydrospiro[pyrrolidine-3,4′-pyrrolo[1,2-b]pyrazole]-1-carboxamide

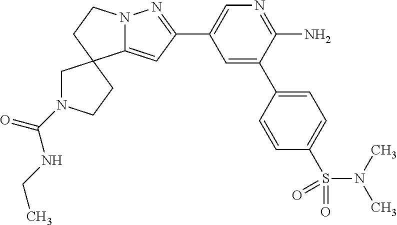 [SMILES: CCNC(=O)N1CCC2(CCn3nc(-c4cnc(N)c(-c5ccc(S(=O)(=O)N(C)C)cc5)c4)cc32)C1]

Example 503 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (100 mg, 262 μmol) and {6-amino-5-[4-(dimethylsulfamoyl)phenyl]pyridin-3-yl}boronic acid (126 mg, 392 μmol).

LC-MS (Method 1): Rt=0.91 min; MS (ESIpos): m/z=510 [M+H]⁺

¹H-NMR (400 MHz, DMSO-d6) δ [ppm]: 0.996 (1.12), 1.014 (2.51), 1.031 (1.14), 2.032 (0.25), 2.051 (0.44), 2.062 (0.22), 2.069 (0.20), 2.518 (1.10), 2.539 (16.00), 2.657 (9.92), 3.031 (0.44), 3.045 (0.50), 3.048 (0.49), 3.063 (0.44), 3.370 (0.17), 3.378 (0.25), 3.396 (1.23), 3.423 (0.17), 3.480 (0.23), 4.147 (0.40), 4.165 (0.67), 4.182 (0.39), 5.934 (0.95), 6.132 (0.19), 6.146 (0.39), 6.159 (0.19), 6.415 (1.61), 7.722 (0.80), 7.727 (0.83), 7.739 (0.69), 7.744 (0.30), 7.760 (1.46), 7.796 (1.57), 7.817 (0.70), 8.410 (0.92), 8.416 (0.89).

### Example 504

### (rac)-2′-[6-amino-5-(cyclohexylmethoxy)pyridin-3-yl]-N-ethyl-5′,6′-dihydrospiro[pyrrolidine-3,4′-pyrrolo[1,2-b]pyrazole]-1-carboxamide

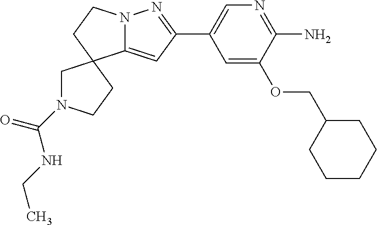 [SMILES: CCNC(=O)N1CCC2(CCn3nc(-c4cnc(N)c(OCC5CCCCC5)c4)cc32)C1]

Example 504 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (60.0 mg, 157 μmol) and [6-amino-5-(cyclohexylmethoxy)pyridin-3-yl]boronic acid (58.9 mg, 235 μmol).

LC-MS (Method 1): Rt=1.12 min; MS (ESIpos): m/z=439 [M+H]⁺

¹H-NMR (400 MHz, DMSO-d6) δ [ppm]: 1.000 (6.94), 1.018 (16.00), 1.036 (7.44), 1.056 (1.46), 1.085 (1.74), 1.114 (0.86), 1.170 (0.61), 1.200 (0.96), 1.222 (2.20), 1.250 (1.79), 1.281 (1.19), 1.643 (0.81), 1.671 (1.01), 1.707 (1.97), 1.738 (1.72), 1.771 (0.86), 1.781 (0.71), 1.845 (1.84), 1.874 (1.74), 2.024 (1.21), 2.038 (1.92), 2.053 (2.57), 2.071 (1.06), 2.083 (0.50), 2.518 (6.69), 2.523 (4.49), 2.527 (3.26), 3.017 (0.91), 3.035 (2.80), 3.050 (3.10), 3.053 (3.00), 3.067 (2.73), 3.084 (0.81), 3.357 (0.68), 3.376 (1.41), 3.395 (8.88), 3.421 (0.91), 3.466 (0.73), 3.484 (1.24), 3.498 (0.93), 3.524 (0.43), 3.795 (5.27), 3.811 (5.05), 4.137 (2.52), 4.155 (4.29), 4.172 (2.50), 5.705 (4.11), 6.138 (1.14), 6.152 (2.25), 6.165 (1.11), 6.347 (10.45), 7.282 (4.14), 7.287 (4.11), 7.898 (5.65), 7.902 (5.50).

### Example 505

### (rac)-2′-{6-amino-5-[(pyridin-3-yl)methoxy]pyridin-3-yl}-N-ethyl-5′,6′-dihydrospiro[pyrrolidine-3,4′-pyrrolo[1,2-b]pyrazole]-1-carboxamide

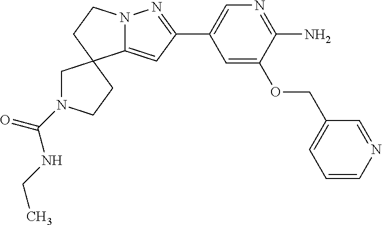 [SMILES: CCNC(=O)N1CCC2(CCn3nc(-c4cnc(N)c(OCc5cccnc5)c4)cc32)C1]

Example 505 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (100 mg, 262 μmol) and {6-amino-5-[(pyridin-3-yl)methoxy]pyridin-3-yl}boronic acid (96.1 mg, 392 μmol).

LC-MS (Method 1): Rt=0.77 min; MS (ESIpos): m/z=434 [M+H]⁺

¹H-NMR (400 MHz, DMSO-d6) δ [ppm]: 1.003 (7.09), 1.021 (16.00), 1.039 (7.25), 1.065 (3.57), 1.155 (5.11), 1.232 (0.58), 1.292 (0.70), 2.040 (1.84), 2.058 (3.50), 2.074 (2.57), 2.083 (0.79), 2.331 (0.96), 2.522 (4.52), 2.532 (4.59), 2.549 (2.80), 2.562 (1.07), 2.673 (0.98), 3.021 (0.96), 3.039 (2.92), 3.053 (3.27), 3.057 (3.20), 3.070 (2.85), 3.088 (0.93), 3.405 (9.66), 3.424 (3.55), 3.434 (2.12), 3.442 (1.87), 3.462 (1.49), 3.480 (2.19), 3.496 (1.40), 3.506 (1.38), 3.521 (0.75), 4.165 (2.59), 4.182 (4.15), 4.200 (2.50), 5.270 (0.77), 5.301 (9.19), 6.153 (1.19), 6.167 (2.36), 6.180 (1.21), 6.437 (8.42), 6.549 (0.58), 6.659 (0.49), 6.720 (0.42), 7.438 (1.80), 7.450 (2.01), 7.458 (1.98), 7.470 (1.94), 7.639 (3.31), 7.937 (5.41), 7.941 (5.32), 7.968 (1.35), 7.972 (2.17), 7.977 (1.45), 7.987 (1.31), 7.992 (2.01), 7.997 (1.28), 8.557 (2.38), 8.560 (2.52), 8.569 (2.43), 8.572 (2.40), 8.761 (3.29), 8.765 (3.29).

### Example 506

### (rac)-2′-{6-amino-5-[(4-fluorophenyl)methoxy]pyridin-3-yl}-N-ethyl-5′,6′-dihydrospiro[pyrrolidine-3,4′-pyrrolo[1,2-b]pyrazole]-1-carboxamide

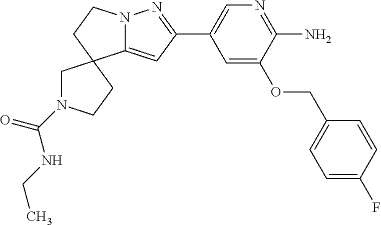 [SMILES: CCNC(=O)N1CCC2(CCn3nc(-c4cnc(N)c(OCc5ccc(F)cc5)c4)cc32)C1]

Example 506 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (100 mg, 262 μmol) and {6-amino-5-[(4-fluorophenyl)methoxy]pyridin-3-yl}boronic acid (103 mg, 392 μmol).

LC-MS (Method 1): Rt=0.98 min; MS (ESIpos): m/z=451 [M+H]⁺

¹H-NMR (400 MHz, DMSO-d6) δ [ppm]: 1.002 (7.09), 1.020 (16.00), 1.038 (7.33), 1.233 (2.30), 1.256 (1.07), 1.295 (0.48), 2.028 (1.35), 2.042 (2.10), 2.055 (2.77), 2.073 (1.23), 2.084 (0.75), 2.522 (8.24), 2.530 (4.36), 3.020 (0.91), 3.038 (2.97), 3.052 (3.37), 3.055 (3.25), 3.069 (2.93), 3.087 (0.87), 3.382 (2.06), 3.398 (10.22), 3.425 (1.23), 3.465 (0.83), 3.483 (1.43), 3.497 (1.03), 3.505 (0.95), 3.523 (0.51), 4.141 (2.69), 4.159 (4.51), 4.176 (2.61), 5.158 (9.54), 5.795 (6.46), 6.140 (1.27), 6.154 (2.46), 6.168 (1.23), 6.339 (10.14), 7.202 (3.52), 7.207 (1.31), 7.224 (7.01), 7.246 (3.96), 7.433 (4.44), 7.437 (4.44), 7.562 (3.29), 7.577 (3.72), 7.584 (3.41), 7.598 (2.85), 7.937 (4.08), 7.941 (3.92).

### Example 507

### 2′-(6-amino-5-{[1-phenylethyl]oxy}pyridin-3-yl)-N-ethyl-5′,6′-dihydrospiro[pyrrolidine-3,4′-pyrrolo[1,2-b]pyrazole]-1-carboxamide (Diastereomeric Mixture)

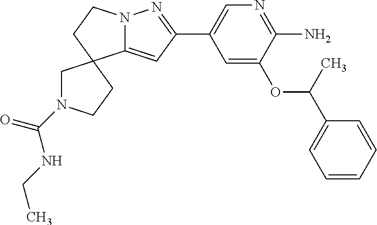 [SMILES: CCNC(=O)N1CCC2(CCn3nc(-c4cnc(N)c(OC(C)c5ccccc5)c4)cc32)C1]

Example 507 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (100 mg, 262 μmol) and (rac)-[6-amino-5-(1-phenylethoxy)pyridin-3-yl]boronic acid (101 mg, 392 μmol).

LC-MS (Method 1): Rₜ=1.00 min; MS (ESIpos): m/z=447 [M+H]⁺

¹H-NMR (400 MHz, DMSO-d6) δ [ppm]: 0.997 (6.32), 1.002 (6.79), 1.015 (14.27), 1.020 (14.84), 1.033 (6.57), 1.038 (6.68), 1.561 (16.00), 1.577 (16.00), 2.010 (2.60), 2.023 (3.25), 2.028 (3.18), 2.041 (1.48), 2.463 (3.83), 2.482 (7.95), 2.518 (6.14), 2.523 (4.05), 3.019 (0.94), 3.032 (3.29), 3.036 (2.93), 3.046 (3.14), 3.050 (4.95), 3.064 (2.82), 3.068 (3.25), 3.081 (0.90), 3.086 (0.83), 3.355 (2.31), 3.373 (11.16), 3.386 (2.35), 3.399 (1.12), 3.445 (1.19), 3.463 (2.13), 3.469 (1.30), 3.477 (1.52), 3.484 (1.26), 3.502 (0.76), 4.104 (4.26), 4.122 (7.19), 4.139 (4.15), 5.558 (0.98), 5.574 (3.47), 5.589 (3.43), 5.605 (1.01), 5.829 (10.73), 6.131 (1.73), 6.145 (3.40), 6.158 (1.66), 6.219 (10.33), 6.225 (9.64), 7.224 (1.59), 7.231 (4.59), 7.235 (7.55), 7.240 (6.25), 7.260 (2.89), 7.264 (2.89), 7.319 (4.44), 7.337 (7.77), 7.339 (7.80), 7.357 (4.26), 7.454 (8.70), 7.472 (6.72), 7.839 (5.45), 7.843 (8.81), 7.847 (5.67).

### Example 508

### 2-{6-amino-5-[(1R)-1-(2,6-dichloro-3-fluorophenyl)ethoxy]pyridin-3-yl}-N-ethyl-6,7-dihydrospiro[pyrazolo[5,1-c][1,4]oxazine-4,3′-pyrrolidine]-1′-carboxamide (Mixture of Stereoisomers)

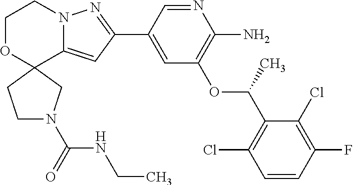 [SMILES: CCNC(=O)N1CCC2(C1)OCCn1nc(-c3cnc(N)c(O[C@H](C)c4c(Cl)ccc(F)c4Cl)c3)cc12]

Example 508 was prepared in analogy to Example 461 using (rac)-2-bromo-N-ethyl-6,7-dihydrospiro[pyrazolo[5,1-c][1,4]oxazine-4,3′-pyrrolidine]-1′-carboxamide (100 mg, 304 μmol) and 3-[(1R)-1-(2,6-dichloro-3-fluorophenyl)ethoxy]-5-(4,4,5,5-tetramethyl-1,3,2-dioxaborolan-2-yl)pyridin-2-amine (156 mg, 365 μmol).

LC-MS (Method 1): Rt=1.11 min; MS (ESIpos): m/z=549 [M+H]⁺

¹H-NMR (400 MHz, DMSO-d6) δ [ppm]: 0.998 (6.00), 1.002 (6.59), 1.016 (13.76), 1.020 (14.82), 1.034 (6.28), 1.038 (6.69), 1.245 (1.19), 1.254 (0.88), 1.261 (1.27), 1.268 (1.22), 1.273 (0.53), 1.284 (1.13), 1.780 (15.99), 1.798 (16.00), 2.074 (3.94), 2.148 (0.51), 2.171 (1.02), 2.180 (1.15), 2.197 (0.93), 2.205 (1.63), 2.229 (0.91), 2.278 (1.28), 2.294 (1.47), 2.311 (0.88), 2.327 (1.12), 2.518 (1.32), 2.523 (0.85), 2.539 (7.74), 2.669 (0.53), 3.016 (0.73), 3.020 (0.87), 3.034 (2.86), 3.038 (2.69), 3.048 (2.80), 3.052 (4.25), 3.056 (2.98), 3.065 (2.54), 3.070 (2.91), 3.083 (0.83), 3.088 (0.78), 3.226 (0.48), 3.249 (0.61), 3.373 (7.30), 3.403 (4.72), 3.494 (1.36), 3.515 (2.34), 3.539 (1.08), 3.719 (2.22), 3.747 (1.93), 4.010 (0.50), 4.024 (1.40), 4.047 (2.24), 4.059 (6.60), 4.065 (6.69), 4.072 (6.24), 4.093 (1.00), 4.100 (1.40), 4.232 (0.66), 5.915 (3.36), 6.033 (0.58), 6.038 (0.66), 6.050 (2.08), 6.055 (2.18), 6.067 (2.12), 6.072 (2.19), 6.083 (0.67), 6.089 (0.60), 6.169 (1.66), 6.183 (3.33), 6.196 (1.66), 6.433 (13.08), 6.440 (2.05), 7.130 (3.50), 7.135 (4.09), 7.138 (4.09), 7.142 (3.66), 7.412 (1.89), 7.416 (1.83), 7.434 (3.67), 7.438 (3.56), 7.456 (2.58), 7.460 (2.58), 7.535 (1.88), 7.541 (1.97), 7.547 (2.17), 7.554 (2.05), 7.557 (1.77), 7.563 (1.56), 7.570 (1.55), 7.576 (1.35), 7.876 (7.59), 7.880 (7.68).

### Example 509

### (rac)-2′-[6-amino-5-(propan-2-yl)pyridin-3-yl]-N-ethyl-5′,6′-dihydrospiro[pyrrolidine-3,4′-pyrrolo[1,2-b]pyrazole]-1-carboxamide

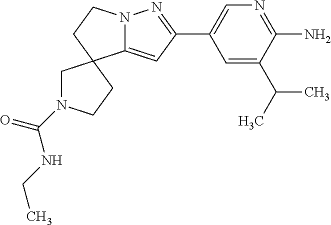 [SMILES: CCNC(=O)N1CCC2(CCn3nc(-c4cnc(N)c(C(C)C)c4)cc32)C1]

Example 509 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (75.0 mg, 196 μmol) and 3-(propan-2-yl)-5-(4,4,5,5-tetramethyl-1,3,2-dioxaborolan-2-yl)pyridin-2-amine (77.1 mg, 294 μmol).

LC-MS (Method 1): Rt=0.86 min; MS (ESIpos): m/z=369 [M+H]⁺

¹H-NMR (400 MHz, DMSO-d6) δ [ppm]: 1.001 (5.11), 1.019 (12.03), 1.037 (5.40), 1.065 (5.22), 1.133 (0.54), 1.150 (0.80), 1.163 (16.00), 1.179 (15.97), 2.024 (0.90), 2.038 (1.60), 2.055 (2.07), 2.073 (0.78), 2.085 (0.41), 2.523 (2.04), 2.529 (2.50), 2.907 (0.91), 2.923 (1.23), 2.940 (0.87), 3.018 (0.67), 3.036 (2.09), 3.050 (2.31), 3.053 (2.29), 3.068 (2.06), 3.086 (0.63), 3.374 (1.54), 3.381 (1.30), 3.397 (7.86), 3.417 (0.76), 3.468 (0.59), 3.486 (0.96), 3.492 (0.61), 3.500 (0.71), 3.507 (0.59), 4.146 (1.96), 4.163 (3.30), 4.181 (1.90), 6.046 (1.41), 6.145 (0.87), 6.159 (1.67), 6.173 (0.83), 6.344 (7.45), 7.679 (2.70), 7.685 (2.72), 8.161 (4.00), 8.166 (3.78).

### Example 510

### (rac)-2-[6-amino-5-(methylcarbamoyl)pyridin-3-yl]-N-ethyl-6,7-dihydrospiro[pyrazolo[5,1-c][1,4]oxazine-4,3′-pyrrolidine]-1′-carboxamide

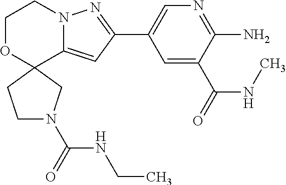 [SMILES: CCNC(=O)N1CCC2(C1)OCCn1nc(-c3cnc(N)c(C(=O)NC)c3)cc12]

Example 510 was prepared in analogy to Example 461 using (rac)-2-bromo-N-ethyl-6,7-dihydrospiro[pyrazolo[5,1-c][1,4]oxazine-4,3′-pyrrolidine]-1′-carboxamide (75.0 mg, 228 μmol) and 2-amino-N-methyl-5-(4,4,5,5-tetramethyl-1,3,2-dioxaborolan-2-yl)pyridine-3-carboxamide (75.8 mg, 273 μmol).

LC-MS (Method 1): Rt=0.69 min; MS (ESIpos): m/z=400 [M+H]⁺

¹H-NMR (400 MHz, DMSO-d6) δ [ppm]: 1.002 (7.02), 1.020 (16.00), 1.038 (7.46), 2.208 (0.95), 2.233 (1.25), 2.256 (0.72), 2.326 (1.63), 2.341 (1.32), 2.358 (0.85), 2.375 (0.71), 2.756 (9.25), 3.019 (0.90), 3.037 (2.89), 3.051 (3.46), 3.069 (2.91), 3.086 (0.92), 3.365 (2.07), 3.376 (1.44), 3.392 (0.88), 3.411 (3.03), 3.440 (3.42), 3.521 (1.06), 3.544 (1.88), 3.566 (0.86), 3.764 (2.10), 3.792 (1.76), 4.051 (0.70), 4.066 (1.31), 4.082 (1.47), 4.099 (1.95), 4.122 (4.28), 4.133 (5.69), 6.171 (1.33), 6.185 (2.65), 6.199 (1.32), 6.623 (10.23), 7.167 (5.09), 8.197 (4.67), 8.203 (4.87), 8.456 (5.89), 8.462 (5.69), 8.542 (1.52).

### Example 511

### (rac)-2′-[6-amino-5-(methylcarbamoyl)pyridin-3-yl]-N-ethyl-5′,6′-dihydrospiro[pyrrolidine-3,4′-pyrrolo[1,2-b]pyrazole]-1-carboxamide

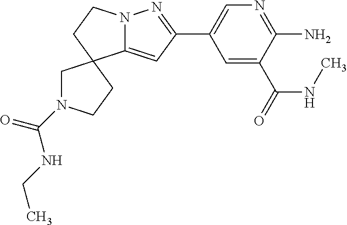 [SMILES: CCNC(=O)N1CCC2(CCn3nc(-c4cnc(N)c(C(=O)NC)c4)cc32)C1]

Example 511 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (75.0 mg, 196 μmol) and 2-amino-N-methyl-5-(4,4,5,5-tetramethyl-1,3,2-dioxaborolan-2-yl)pyridine-3-carboxamide (65.2 mg, 235 μmol).

LC-MS (Method 1): Rt=0.69 min; MS (ESIpos): m/z=384 [M+H]⁺

¹H-NMR (400 MHz, DMSO-d6) δ [ppm]: 0.988 (0.42), 1.005 (7.74), 1.022 (16.00), 1.041 (7.27), 2.050 (1.88), 2.068 (4.12), 2.085 (2.02), 2.518 (6.85), 2.523 (4.35), 2.539 (2.99), 2.557 (3.91), 2.574 (2.61), 2.781 (10.82), 2.793 (10.94), 2.801 (2.26), 2.812 (1.69), 3.042 (2.05), 3.055 (2.38), 3.072 (1.95), 3.320 (0.61), 3.411 (5.65), 3.418 (6.05), 3.443 (2.94), 3.452 (1.36), 3.470 (2.09), 3.487 (1.11), 3.495 (1.01), 3.512 (0.45), 3.931 (0.49), 4.151 (0.80), 4.188 (2.89), 4.206 (4.40), 4.223 (2.78), 6.178 (1.29), 6.465 (9.04), 6.691 (0.61), 6.962 (0.66), 7.090 (0.75), 7.217 (0.66), 8.447 (0.71), 8.454 (0.92), 8.482 (3.60), 8.488 (6.14), 8.499 (3.01), 8.834 (1.15), 8.846 (1.15).

### Example 512

### (rac)-2′-(6-amino-5-cyclopropylpyridin-3-yl)-N-ethyl-5′,6′-dihydrospiro[pyrrolidine-3,4′-pyrrolo[1,2-b]pyrazole]-1-carboxamide

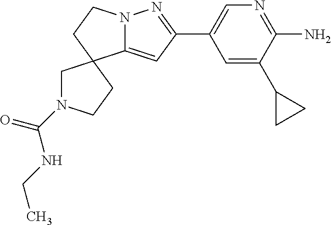 [SMILES: CCNC(=O)N1CCC2(CCn3nc(-c4cnc(N)c(C5CC5)c4)cc32)C1]

Example 512 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (75.0 mg, 196 μmol) and 3-cyclopropyl-5-(4,4,5,5-tetramethyl-1,3,2-dioxaborolan-2-yl)pyridin-2-amine (76.5 mg, 294 μmol).

LC-MS (Method 1): Rt=0.81 min; MS (ESIpos): m/z=367 [M+H]⁺

¹H-NMR (400 MHz, DMSO-d6) δ [ppm]: 0.570 (0.93), 0.581 (2.77), 0.585 (2.92), 0.594 (3.27), 0.599 (2.78), 0.609 (1.08), 0.889 (0.97), 0.899 (2.78), 0.904 (2.57), 0.910 (1.45), 0.914 (1.46), 0.920 (2.78), 0.924 (2.66), 0.935 (0.93), 1.000 (5.84), 1.018 (13.69), 1.036 (6.19), 1.691 (0.70), 1.698 (0.77), 1.711 (1.29), 1.724 (0.71), 1.732 (0.63), 2.024 (1.28), 2.043 (2.38), 2.054 (1.15), 2.061 (1.07), 2.527 (2.61), 2.539 (16.00), 3.017 (0.79), 3.035 (2.48), 3.049 (2.75), 3.053 (2.69), 3.067 (2.45), 3.084 (0.77), 3.362 (1.57), 3.376 (2.02), 3.392 (6.06), 3.401 (2.54), 3.418 (1.46), 3.461 (0.87), 3.479 (1.39), 3.487 (0.86), 3.494 (0.96), 3.504 (0.89), 3.520 (0.51), 4.139 (2.28), 4.157 (3.79), 4.174 (2.22), 6.137 (0.98), 6.151 (1.98), 6.165 (0.99), 6.335 (1.12), 6.371 (8.91), 7.509 (2.95), 7.514 (2.96), 8.134 (5.19), 8.165 (3.53), 8.170 (3.53).

### Example 513

### (rac)-2′-[6-amino-5-(methylcarbamoyl)pyridin-3-yl]-N-(1-phenylcyclobutyl)-5′,6′-dihydrospiro[pyrrolidine-3,4′-pyrrolo[1,2-b]pyrazole]-1-carboxamide

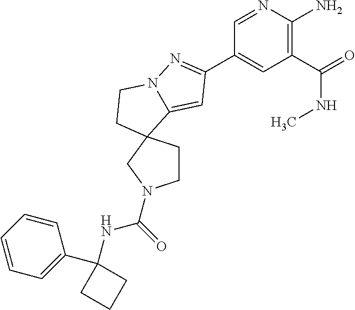 [SMILES: CNC(=O)c1cc(-c2cc3n(n2)CCC32CCN(C(=O)NC3(c4ccccc4)CCC3)C2)cnc1N]

Example 513 was prepared in analogy to Example 461 using (rac)-1-[(1-phenylcyclobutyl)carbamoyl]-5′,6′-dihydrospiro[pyrrolidine-3,4′-pyrrolo[1,2-b]pyrazol]-2′-yl trifluoromethanesulfonate (194 mg, 400 μmol) and 2-amino-N-methyl-5-(4,4,5,5-tetramethyl-1,3,2-dioxaborolan-2-yl)pyridine-3-carboxamide (127 mg, 460 μmol).

LC-MS (Method 1): Rt=0.98 min; MS (ESIpos): m/z=486 [M+H]⁺

¹H-NMR (400 MHz, DMSO-d6) δ [ppm]: 1.232 (0.21), 1.760 (0.23), 1.766 (0.24), 1.783 (0.25), 1.788 (0.27), 1.805 (0.17), 1.975 (0.26), 1.990 (0.21), 2.002 (0.24), 2.054 (0.48), 2.071 (0.95), 2.088 (0.50), 2.384 (0.41), 2.394 (0.50), 2.406 (0.53), 2.428 (0.29), 2.451 (0.35), 2.475 (0.72), 2.522 (1.02), 2.539 (16.00), 2.560 (1.05), 2.576 (0.68), 2.791 (2.99), 2.801 (3.02), 3.413 (0.19), 3.441 (1.26), 3.466 (0.53), 3.484 (0.26), 3.509 (0.42), 3.526 (0.26), 3.535 (0.24), 4.197 (0.68), 4.215 (1.27), 4.232 (0.82), 6.472 (2.40), 6.718 (1.08), 6.984 (0.41), 7.112 (0.48), 7.133 (0.34), 7.151 (0.86), 7.170 (0.57), 7.240 (0.51), 7.263 (0.97), 7.284 (1.64), 7.302 (0.95), 7.422 (1.58), 7.440 (1.26), 7.443 (1.01), 8.466 (1.16), 8.471 (1.29), 8.568 (1.05), 8.572 (0.99), 8.921 (0.42), 8.932 (0.43).

### Example 514

### (rac)-2′-[6-amino-5-(dimethylcarbamoyl)pyridin-3-yl]-N-(1-phenylcyclobutyl)-5′,6′-dihydrospiro[pyrrolidine-3,4′-pyrrolo[1,2-b]pyrazole]-1-carboxamide

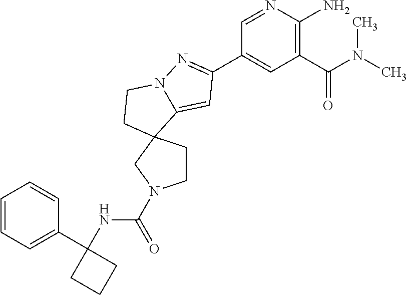 [SMILES: CN(C)C(=O)c1cc(-c2cc3n(n2)CCC32CCN(C(=O)NC3(c4ccccc4)CCC3)C2)cnc1N]

Example 514 was prepared in analogy to Example 461 using (rac)-1-[(1-phenylcyclobutyl)carbamoyl]-5′,6′-dihydrospiro[pyrrolidine-3,4′-pyrrolo[1,2-b]pyrazol]-2′-yl trifluoromethanesulfonate (169 mg, 348 μmol) and 2-amino-N,N-dimethyl-5-(4,4,5,5-tetramethyl-1,3,2-dioxaborolan-2-yl)pyridine-3-carboxamide (116 mg, 400 μmol).

LC-MS (Method 1): Rt=0.98 min; MS (ESIpos): m/z=500 [M+H]⁺

¹H-NMR (400 MHz, DMSO-d6) δ [ppm]: 1.746 (0.67), 1.763 (1.25), 1.768 (1.32), 1.773 (0.92), 1.779 (0.92), 1.784 (1.32), 1.790 (1.55), 1.807 (0.92), 1.956 (0.65), 1.963 (0.82), 1.979 (1.50), 1.994 (1.20), 2.002 (1.47), 2.006 (1.55), 2.017 (1.20), 2.029 (2.27), 2.043 (3.05), 2.056 (3.32), 2.074 (2.45), 2.086 (0.67), 2.356 (0.95), 2.383 (2.45), 2.394 (2.25), 2.399 (2.75), 2.415 (1.32), 2.421 (1.55), 2.447 (1.52), 2.469 (3.32), 2.475 (2.87), 2.518 (10.51), 2.522 (7.66), 2.539 (7.41), 2.941 (12.06), 3.375 (0.90), 3.391 (2.55), 3.400 (1.70), 3.415 (5.97), 3.432 (4.89), 3.457 (1.47), 3.504 (0.92), 3.521 (1.70), 3.529 (1.20), 3.537 (1.25), 3.546 (1.20), 4.150 (3.94), 4.167 (7.49), 4.185 (3.87), 6.058 (10.83), 6.326 (16.00), 6.694 (7.16), 7.126 (1.20), 7.129 (2.27), 7.133 (1.27), 7.143 (1.57), 7.148 (5.32), 7.152 (1.82), 7.163 (2.10), 7.166 (3.57), 7.169 (2.02), 7.257 (6.04), 7.261 (2.40), 7.277 (9.68), 7.290 (1.87), 7.295 (5.67), 7.416 (8.26), 7.419 (9.04), 7.432 (2.25), 7.437 (7.44), 7.440 (5.79), 7.659 (9.19), 7.665 (9.49), 8.135 (2.62), 8.383 (11.13), 8.388 (10.81).

### Example 515

### 2′-{6-amino-5-[(1R)-1-(pyridin-4-yl)ethoxy]pyridin-3-yl}-N-ethyl-5′,6′-dihydrospiro[pyrrolidine-3,4′-pyrrolo[1,2-b]pyrazole]-1-carboxamide (Mixture of Stereoisomers)

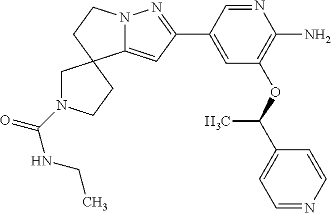 [SMILES: CCNC(=O)N1CCC2(CCn3nc(-c4cnc(N)c(O[C@H](C)c5ccncc5)c4)cc32)C1]

Example 515 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (59.0 mg, 154 μmol) and {6-amino-5-[(1R)-1-(pyridin-4-yl)ethoxy]pyridin-3-yl}boronic acid (60.0 mg, 232 μmol).

LC-MS (Method 1): Rt=0.79 min; MS (ESIpos): m/z=448 [M+H]⁺

¹H-NMR (400 MHz, DMSO-d6) δ [ppm]: 0.851 (1.66), 0.995 (6.95), 0.999 (7.34), 1.013 (15.45), 1.016 (15.94), 1.031 (7.28), 1.035 (8.61), 1.052 (3.42), 1.070 (2.15), 1.232 (8.77), 1.255 (1.99), 1.334 (1.49), 1.347 (1.27), 1.554 (14.73), 1.570 (14.68), 1.900 (1.99), 2.022 (3.53), 2.463 (4.47), 2.518 (16.00), 2.523 (9.82), 3.015 (1.21), 3.032 (3.64), 3.047 (5.35), 3.064 (3.86), 3.082 (1.21), 3.164 (2.32), 3.443 (2.81), 3.461 (3.37), 4.103 (3.97), 4.121 (6.46), 4.137 (3.86), 5.649 (3.03), 5.665 (3.03), 5.928 (9.49), 6.133 (1.77), 6.147 (3.64), 6.160 (2.15), 6.180 (3.09), 6.245 (10.21), 6.247 (9.38), 7.239 (6.29), 7.474 (8.83), 7.490 (9.27), 7.874 (5.08), 7.878 (7.89), 7.882 (4.86), 8.376 (1.99), 8.529 (7.34), 8.532 (8.06), 8.545 (7.94), 8.547 (6.34).

### Example 516

### 2′-{6-amino-5-[(1S)-1-(pyridin-4-yl)ethoxy]pyridin-3-yl}-N-ethyl-5′,6′-dihydrospiro[pyrrolidine-3,4′-pyrrolo[1,2-b]pyrazole]-1-carboxamide (Mixture of Stereoisomers)

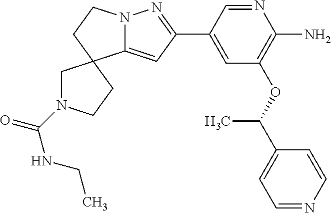 [SMILES: CCNC(=O)N1CCC2(CCn3nc(-c4cnc(N)c(O[C@@H](C)c5ccncc5)c4)cc32)C1]

Example 516 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (98.4 mg, 257 μmol) and {6-amino-5-[(1S)-1-(pyridin-4-yl)ethoxy]pyridin-3-yl}boronic acid (100 mg, 386 μmol).

LC-MS (Method 1): Rt=0.79 min; MS (ESIpos): m/z=448 [M+H]⁺

¹H-NMR (400 MHz, DMSO-d6) δ [ppm]: 0.996 (6.71), 0.999 (6.97), 1.014 (14.82), 1.017 (14.97), 1.031 (7.21), 1.035 (7.15), 1.066 (0.47), 1.495 (0.53), 1.554 (15.82), 1.570 (16.00), 2.022 (3.65), 2.028 (3.56), 2.463 (4.74), 3.014 (1.12), 3.031 (3.74), 3.047 (5.38), 3.064 (3.76), 3.079 (1.12), 3.372 (11.97), 3.443 (1.38), 3.461 (2.35), 3.476 (1.76), 3.500 (0.85), 4.103 (4.24), 4.121 (7.32), 4.138 (4.26), 5.633 (1.06), 5.649 (3.47), 5.665 (3.47), 5.681 (1.03), 5.927 (11.12), 6.129 (1.97), 6.143 (3.97), 6.157 (2.06), 6.245 (10.15), 6.247 (10.29), 7.240 (7.06), 7.475 (9.82), 7.490 (10.15), 7.874 (5.21), 7.878 (8.47), 7.882 (5.35), 8.530 (7.97), 8.532 (9.38), 8.544 (9.24), 8.547 (7.94).

### Example 517

### (rac)-2′-[6-amino-5-(1-methyl-1H-pyrazol-3-yl)pyridin-3-yl]-N-ethyl-5′,6′-dihydrospiro[pyrrolidine-3,4′-pyrrolo[1,2-b]pyrazole]-1-carboxamide

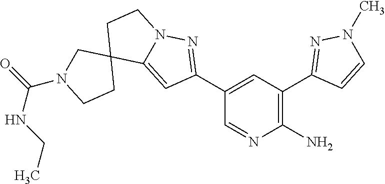 [SMILES: CCNC(=O)N1CCC2(CCn3nc(-c4cnc(N)c(-c5ccn(C)n5)c4)cc32)C1]

Example 517 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (117 mg, 306 μmol) and [6-amino-5-(1-methyl-1H-pyrazol-3-yl)pyridin-3-yl]boronic acid (100 mg, 459 μmol).

LC-MS (Method 1): Rt=0.81 min; MS (ESIpos): m/z=407 [M+H]⁺

¹H-NMR (400 MHz, DMSO-d6) δ [ppm]: 1.005 (4.62), 1.024 (10.37), 1.041 (4.85), 1.865 (5.43), 2.041 (0.96), 2.055 (1.42), 2.067 (1.89), 2.085 (0.84), 2.527 (3.03), 2.539 (14.06), 2.544 (2.36), 3.024 (0.68), 3.042 (2.06), 3.056 (2.34), 3.060 (2.27), 3.074 (2.05), 3.091 (0.67), 3.387 (3.67), 3.392 (3.75), 3.400 (3.29), 3.414 (8.12), 3.436 (1.72), 3.483 (1.00), 3.501 (1.31), 3.508 (0.92), 3.516 (0.98), 3.527 (0.85), 3.541 (0.56), 3.921 (16.00), 4.164 (1.87), 4.181 (3.15), 4.198 (1.80), 6.148 (0.85), 6.162 (1.70), 6.176 (0.86), 6.451 (7.12), 6.870 (3.91), 6.875 (3.99), 7.076 (2.26), 7.801 (3.47), 7.806 (3.42), 8.138 (3.43), 8.143 (3.62), 8.332 (4.09), 8.338 (3.93).

### Example 518

### (rac)-2′-[6-amino-5-(1,2-thiazol-5-yl)pyridin-3-yl]-N-ethyl-5′,6′-dihydrospiro[pyrrolidine-3,4′-pyrrolo[1,2-b]pyrazole]-1-carboxamide

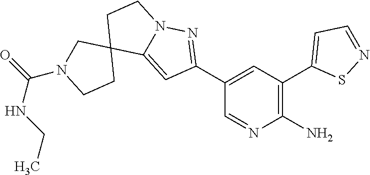 [SMILES: CCNC(=O)N1CCC2(CCn3nc(-c4cnc(N)c(-c5ccns5)c4)cc32)C1]

Example 518 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (138 mg, 362 μmol) and [6-amino-5-(1,2-thiazol-5-yl)pyridin-3-yl]boronic acid (120 mg, 543 μmol).

LC-MS (Method 1): Rt=0.81 min; MS (ESIpos): m/z=410 [M+H]⁺

¹H-NMR (400 MHz, DMSO-d6) δ [ppm]: 1.001 (7.05), 1.010 (0.94), 1.019 (16.00), 1.026 (1.12), 1.037 (7.12), 2.036 (1.28), 2.042 (1.29), 2.051 (1.89), 2.060 (2.53), 2.068 (1.31), 2.078 (1.17), 2.090 (0.47), 2.518 (1.68), 2.525 (3.83), 2.543 (2.73), 3.019 (0.87), 3.037 (2.78), 3.051 (3.16), 3.054 (3.07), 3.069 (2.85), 3.086 (0.89), 3.380 (1.25), 3.385 (1.75), 3.392 (1.57), 3.406 (8.70), 3.429 (1.08), 3.471 (0.84), 3.489 (1.34), 3.497 (0.81), 3.504 (1.08), 3.514 (0.85), 3.530 (0.49), 4.166 (2.54), 4.184 (4.11), 4.201 (2.45), 6.154 (7.57), 6.171 (1.41), 6.466 (10.91), 7.732 (7.30), 7.736 (7.64), 7.931 (5.66), 7.937 (5.71), 8.465 (6.41), 8.471 (6.27), 8.629 (7.51), 8.633 (7.18).

### Example 519

### (rac)-2′-[6-amino-5-(1,5-dimethyl-1H-pyrazol-4-yl)pyridin-3-yl]-N-ethyl-5′,6′-dihydrospiro[pyrrolidine-3,4′-pyrrolo[1,2-b]pyrazole]-1-carboxamide

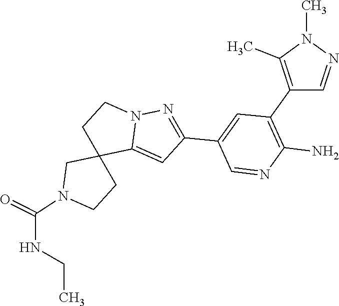 [SMILES: CCNC(=O)N1CCC2(CCn3nc(-c4cnc(N)c(-c5cnn(C)c5C)c4)cc32)C1]

Example 519 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (110 mg, 287 μmol) and [6-amino-5-(1,5-dimethyl-1H-pyrazol-4-yl)pyridin-3-yl]boronic acid (100 mg, 431 μmol).

LC-MS (Method 1): Rt=0.74 min; MS (ESIpos): m/z=421 [M+H]⁺

¹H-NMR (400 MHz, DMSO-d6) δ [ppm]: 0.998 (4.35), 1.016 (10.15), 1.033 (4.62), 2.024 (0.77), 2.036 (1.26), 2.054 (1.71), 2.072 (0.68), 2.171 (15.49), 2.186 (1.19), 2.518 (0.89), 2.524 (1.69), 3.015 (0.55), 3.033 (1.74), 3.047 (1.94), 3.051 (1.88), 3.065 (1.72), 3.083 (0.51), 3.365 (1.14), 3.372 (0.89), 3.396 (5.87), 3.466 (0.45), 3.484 (0.74), 3.491 (0.51), 3.498 (0.57), 3.505 (0.46), 3.509 (0.46), 3.774 (16.00), 4.141 (1.56), 4.159 (2.66), 4.176 (1.50), 5.582 (3.75), 6.134 (0.71), 6.148 (1.45), 6.162 (0.73), 6.344 (6.43), 7.419 (5.94), 7.531 (3.40), 7.537 (3.49), 8.295 (3.83), 8.300 (3.79).

### Example 520

### (rac)-2′-{6-amino-5-[(trifluoromethyl)sulfanyl]pyridin-3-yl}-N-ethyl-5′,6′-dihydrospiro[pyrrolidine-3,4′-pyrrolo[1,2-b]pyrazole]-1-carboxamide

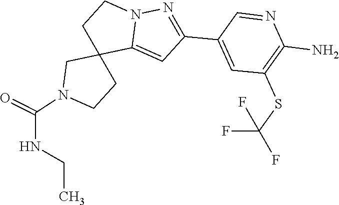 [SMILES: CCNC(=O)N1CCC2(CCn3nc(-c4cnc(N)c(SC(F)(F)F)c4)cc32)C1]

Example 520 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (161 mg, 420 μmol) and {6-amino-5-[(trifluoromethyl)sulfanyl]pyridin-3-yl}boronic acid (150 mg, 630 μmol).

LC-MS (method 1): Rₜ=0.95 min; MS (ESIneg): m/z=425 [M−H]⁻

¹H-NMR (400 MHz, DMSO-d6) δ [ppm]: 1.001 (6.98), 1.019 (16.00), 1.036 (7.52), 1.053 (1.26), 1.070 (0.63), 1.231 (1.75), 2.029 (1.42), 2.035 (1.44), 2.046 (2.04), 2.053 (2.70), 2.071 (1.21), 2.083 (0.44), 2.518 (5.64), 2.536 (2.85), 3.019 (0.91), 3.036 (2.84), 3.050 (3.21), 3.053 (3.10), 3.068 (2.81), 3.086 (0.82), 3.377 (1.88), 3.384 (1.62), 3.398 (10.06), 3.420 (0.98), 3.466 (0.82), 3.484 (1.39), 3.492 (0.84), 3.499 (0.98), 3.509 (0.86), 3.525 (0.48), 4.157 (2.74), 4.175 (4.50), 4.192 (2.63), 6.140 (1.19), 6.153 (2.34), 6.167 (1.16), 6.428 (10.94), 6.736 (6.68), 8.052 (4.00), 8.057 (4.07), 8.526 (6.03), 8.532 (5.80).

### Example 521

### 5-[1-methyl-5′,6′-dihydrospiro[pyrrolidine-3,4′-pyrrolo[1,2-b]pyrazol]-2′-yl]-3-(trifluoromethyl)pyridin-2-amine (Stereoisomer 1)

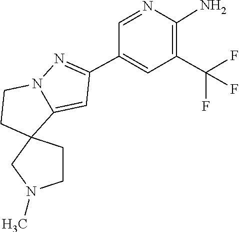 [SMILES: CN1CCC2(CCn3nc(-c4cnc(N)c(C(F)(F)F)c4)cc32)C1]

Example 521 was prepared in analogy to Example 461 using 1-methyl-5′,6′-dihydrospiro[pyrrolidine-3,4′-pyrrolo[1,2-b]pyrazol]-2′-yl trifluoromethanesulfonate (230 mg, 707 μmol, stereoisomer 1) and 5-(4,4,5,5-tetramethyl-1,3,2-dioxaborolan-2-yl)-3-(trifluoromethyl)pyridin-2-amine (306 mg, 1.06 mmol).

1H NMR (DMSO-d6) δ: 8.59 (d, 1H), 8.02 (d, 1H), 6.51 (s, 2H), 6.45 (s, 1H), 4.10 (m, 2H), 2.66-2.74 (m, 2H), 2.53 (br d, 4H), 1.97-2.08 (m, 2H)

[α]²⁰_{D}: −21.8° (c=1.00, DMSO)

LC-MS (method 1): Rₜ=0.97 min; MS (ESIpos): m/z=339 [M+H]⁺

### Example 522

### 5-[1-methyl-5′,6′-dihydrospiro[pyrrolidine-3,4′-pyrrolo[1,2-b]pyrazol]-2′-yl]-3-(trifluoromethyl)pyridin-2-amine (Stereoisomer 2)

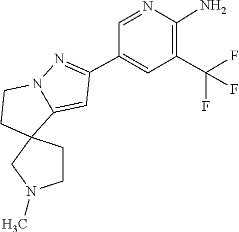 [SMILES: CN1CCC2(CCn3nc(-c4cnc(N)c(C(F)(F)F)c4)cc32)C1]

Example 522 was prepared in analogy to Example 461 using 1-methyl-5′,6′-dihydrospiro[pyrrolidine-3,4′-pyrrolo[1,2-b]pyrazol]-2′-yl trifluoromethanesulfonate (240 mg, 738 μmol, stereoisomer 2) and 5-(4,4,5,5-tetramethyl-1,3,2-dioxaborolan-2-yl)-3-(trifluoromethyl)pyridin-2-amine (319 mg, 1.11 mmol).

1H NMR (DMSO-d6) δ: 8.59 (d, 1H), 8.02 (d, 1H), 6.51 (s, 2H), 6.45 (s, 1H), 4.10 (m, 2H), 2.66-2.75 (m, 2H), 2.51-2.64 (m, 4H), 1.96-2.10 (m, 2H)

[α]²⁰_{D}: +21.6° (c=1.00, DMSO)

LC-MS (method 1): Rₜ=0.97 min; MS (ESIpos): m/z=339 [M+H]⁺

### Example 523

### (3R)-2′-{6-amino-5-[(1R)-1-(2-chlorophenyl)ethoxy]pyridin-3-yl}-N-ethyl-5′,6′-dihydrospiro[pyrrolidine-3,4′-pyrrolo[1,2-b]pyrazole]-1-carboxamide

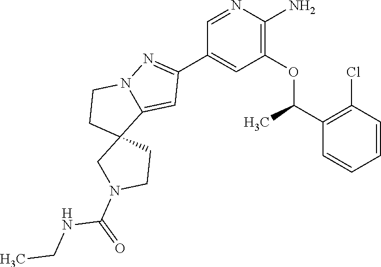 [SMILES: CCNC(=O)N1CC[C@@]2(CCn3nc(-c4cnc(N)c(O[C@H](C)c5ccccc5Cl)c4)cc32)C1]

Example 523 was prepared in analogy to Example 461 using 3-[(1R)-1-(2-chlorophenyl)ethoxy]-5-(4,4,5,5-tetramethyl-1,3,2-dioxaborolan-2-yl)pyridin-2-amine (85.0 mg, 227 μmol) and (3R)-1-(ethylcarbamoyl)-5′,6′-dihydrospiro[pyrrolidine-3,4′-pyrrolo[1,2-b]pyrazol]-2′-yl trifluoromethanesulfonate (91.1 mg, 238 μmol).

1H NMR (DMSO-d6, 400 MHz) δ 7.86 (d, 1H), 7.60 (dd, 1H), 7.47 (dd, 1H), 7.3-7.4 (m, 2H), 7.09 (d, 1H), 6.17 (s, 1H), 6.14 (t, 1H), 5.94 (s, 2H), 5.77 (q, 1H), 4.12 (t, 2H), 3.4-3.5 (m, 1H), 3.3-3.4 (m, 3H), 3.0-3.1 (m, 2H), 2.0-2.1 (m, 2H), 1.60 (d, 3H), 1.01 (t, 3H)

LC-MS (method 1): Rₜ=1.04 min; MS (ESIpos): m/z=482 [M+H]⁺

### Example 524

### 2′-{6-amino-5-[(1R)-1-(2-chlorophenyl)ethoxy]pyridin-3-yl}-N-ethyl-5′,6′-dihydrospiro[azetidine-3,4′-pyrrolo[1,2-b]pyrazole]-1-carboxamide

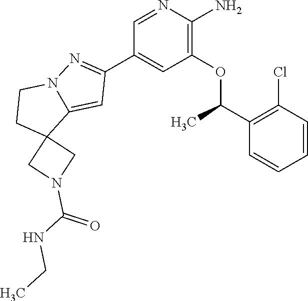 [SMILES: CCNC(=O)N1CC2(CCn3nc(-c4cnc(N)c(O[C@H](C)c5ccccc5Cl)c4)cc32)C1]

Example 524 was prepared in analogy to Example 461 using 3-[(1R)-1-(2-chlorophenyl)ethoxy]-5-(4,4,5,5-tetramethyl-1,3,2-dioxaborolan-2-yl)pyridin-2-amine (85.0 mg, 227 μmol) and 1-(ethylcarbamoyl)-5′,6′-dihydrospiro[azetidine-3,4′-pyrrolo[1,2-b]pyrazol]-2′-yl trifluoromethanesulfonate (87.7 mg, 238 μmol).

1H NMR (DMSO-d6, 400 MHz) δ 7.89 (d, 1H), 7.60 (dd, 1H), 7.46 (dd, 1H), 7.3-7.4 (m, 2H), 7.13 (d, 1H), 6.4-6.5 (m, 2H), 5.96 (s, 2H), 5.78 (q, 1H), 4.0-4.1 (m, 2H), 3.9-4.0 (m, 4H), 3.0-3.1 (m, 2H), 2.79 (t, 2H), 1.60 (d, 3H), 1.0-1.0 (m, 3H)

LC-MS (method 1): Rₜ=1.02 min; MS (ESIpos): m/z=468 [M+H]⁺

### Example 525

### 2′-{6-amino-5-[(1S)-1-(2-chlorophenyl)ethoxy]pyridin-3-yl}-N-ethyl-5′,6′-dihydrospiro[azetidine-3,4′-pyrrolo[1,2-b]pyrazole]-1-carboxamide

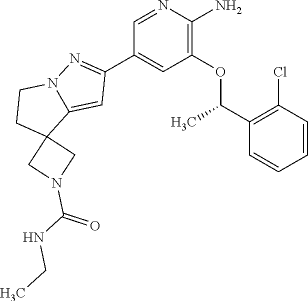 [SMILES: CCNC(=O)N1CC2(CCn3nc(-c4cnc(N)c(O[C@@H](C)c5ccccc5Cl)c4)cc32)C1]

Example 525 was prepared in analogy to Example 461 using 3-[(1S)-1-(2-chlorophenyl)ethoxy]-5-(4,4,5,5-tetramethyl-1,3,2-dioxaborolan-2-yl)pyridin-2-amine (50.0 mg, 133 μmol) and 1-(ethylcarbamoyl)-5′,6′-dihydrospiro[azetidine-3,4′-pyrrolo[1,2-b]pyrazol]-2′-yl trifluoromethanesulfonate (51.6 mg, 140 μmol).

1H NMR (DMSO-d6, 400 MHz) δ 7.89 (d, 1H), 7.60 (dd, 1H), 7.46 (dd, 1H), 7.3-7.4 (m, 2H), 7.13 (d, 1H), 6.4-6.5 (m, 2H), 5.94 (s, 2H), 5.7-5.8 (m, 1H), 4.07 (t, 2H), 3.9-4.0 (m, 4H), 3.02 (dd, 2H), 2.79 (t, 2H), 1.60 (d, 3H), 1.01 (t, 3H)

LC-MS (method 1): Rₜ=1.02 min; MS (ESIpos): m/z=468 [M+H]⁺

### Example 526

### (3′R)-2-{6-amino-5-[(1R)-1-(2,6-dichloro-3-fluorophenyl)ethoxy]pyridin-3-yl}-N-ethyl-6,7-dihydrospiro[pyrazolo[5,1-c][1,4]oxazine-4,3′-pyrrolidine]-1′-carboxamide

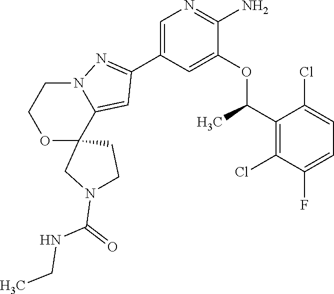 [SMILES: CCNC(=O)N1CC[C@]2(C1)OCCn1nc(-c3cnc(N)c(O[C@H](C)c4c(Cl)ccc(F)c4Cl)c3)cc12]

Example 526 was prepared in analogy to Example 461 using 3-[(1R)-1-(2,6-dichloro-3-fluorophenyl)ethoxy]-5-(4,4,5,5-tetramethyl-1,3,2-dioxaborolan-2-yl)pyridin-2-amine (140 mg, 328 μmol) and (3′R)-2-bromo-N-ethyl-6,7-dihydrospiro[pyrazolo[5,1-c][1,4]oxazine-4,3′-pyrrolidine]-1′-carboxamide (119 mg, 361 μmol).

1H NMR (DMSO-d6, 400 MHz) δ 7.88 (d, 1H), 7.56 (dd, 1H), 7.4-7.5 (m, 1H), 7.12 (d, 1H), 6.43 (s, 1H), 6.17 (t, 1H), 6.05 (q, 1H), 5.83 (s, 2H), 4.0-4.1 (m, 4H), 3.73 (d, 1H), 3.5-3.6 (m, 1H), 3.39 (d, 1H), 3.0-3.1 (m, 2H), 2.3-2.3 (m, 1H), 2.1-2.2 (m, 1H), 1.79 (d, 3H), 1.02 (t, 3H)

LC-MS (method 1): Rₜ=1.09 min; MS (ESIpos): m/z=549 [M+H]⁺

### Example 527

### (3R)-2′-{6-amino-5-[(1R)-1-(pyridin-2-yl)ethoxy]pyridin-3-yl}-N-ethyl-5′,6′-dihydrospiro[pyrrolidine-3,4′-pyrrolo[1,2-b]pyrazole]-1-carboxamide

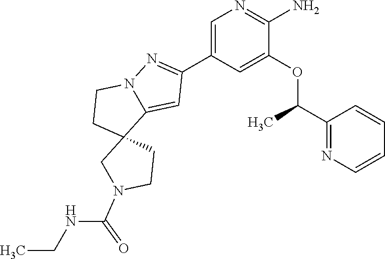 [SMILES: CCNC(=O)N1CC[C@@]2(CCn3nc(-c4cnc(N)c(O[C@H](C)c5ccccn5)c4)cc32)C1]

Example 527 was prepared in analogy to Example 461 using 3-[(1R)-1-(pyridin-2-yl)ethoxy]-5-(4,4,5,5-tetramethyl-1,3,2-dioxaborolan-2-yl)pyridin-2-amine (220 mg, 645 μmol) and (3R)-1-(ethylcarbamoyl)-5′,6′-dihydrospiro[pyrrolidine-3,4′-pyrrolo[1,2-b]pyrazol]-2′-yl trifluoromethanesulfonate (222 mg, 580 μmol).

1H NMR (DMSO-d6, 400 MHz) δ 8.7-8.7 (m, 1H), 8.47 (dd, 1H), 7.91 (td, 1H), 7.87 (d, 1H), 7.38 (ddd, 1H), 7.33 (d, 1H), 6.27 (s, 1H), 6.15 (t, 1H), 5.89 (s, 2H), 5.71 (q, 1H), 4.13 (t, 2H), 3.4-3.5 (m, 1H), 3.4-3.4 (m, 3H), 3.0-3.1 (m, 2H), 2.0-2.1 (m, 2H), 1.60 (d, 3H), 1.02 (t, 3H)

LC-MS (method 1): Rₜ=0.80 min; MS (ESIpos): m/z=449 [M+H]⁺

### Example 528

### 2′-{6-amino-5-[(1R)-1-(pyridin-3-yl)ethoxy]pyridin-3-yl}-N-ethyl-5′,6′-dihydrospiro[azetidine-3,4′-pyrrolo[1,2-b]pyrazole]-1-carboxamide

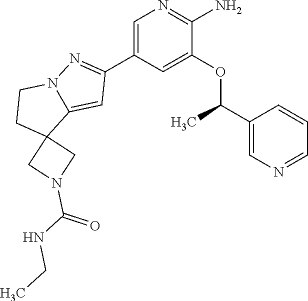 [SMILES: CCNC(=O)N1CC2(CCn3nc(-c4cnc(N)c(O[C@H](C)c5cccnc5)c4)cc32)C1]

Example 528 was prepared in analogy to Example 461 using 3-[(1R)-1-(pyridin-3-yl)ethoxy]-5-(4,4,5,5-tetramethyl-1,3,2-dioxaborolan-2-yl)pyridin-2-amine (40.0 mg, 117 μmol) and 1-(ethylcarbamoyl)-5′,6′-dihydrospiro[azetidine-3,4′-pyrrolo[1,2-b]pyrazol]-2′-yl trifluoromethanesulfonate (47.5 mg, 129 μmol).

1H NMR (DMSO-d6, 400 MHz) δ 8.4-8.6 (m, 1H), 7.90 (d, 1H), 7.79 (dt, 1H), 7.51 (d, 1H), 7.29 (ddd, 1H), 7.24 (d, 1H), 6.48 (s, 1H), 6.44 (t, 1H), 5.89 (s, 2H), 5.53 (q, 1H), 4.06 (t, 2H), 3.9-4.0 (m, 4H), 3.0-3.1 (m, 2H), 2.79 (t, 2H), 1.62 (d, 3H), 1.01 (t, 3H)

LC-MS (method 1): Rₜ=0.81 min; MS (ESIpos): m/z=435 [M+H]⁺

### Example 529

### (3R)-2′-{6-amino-5-[1-(pyrimidin-5-yl)ethoxy]pyridin-3-yl}-N-ethyl-5′,6′-dihydro-1H-spiro[pyrrolidine-3,4′-pyrrolo[1,2-b]pyrazole]-1-carboxamide (Isomer 1)

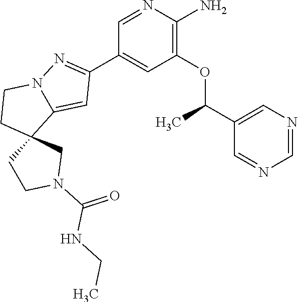 [SMILES: CCNC(=O)N1CC[C@@]2(CCn3nc(-c4cnc(N)c(O[C@H](C)c5cncnc5)c4)cc32)C1]

Example 529 was prepared in analogy to Example 461 using (3R)-1-(ethylcarbamoyl)-5′,6′-dihydrospiro[pyrrolidine-3,4′-pyrrolo[1,2-b]pyrazol]-2′-yl trifluoromethanesulfonate (98.8 mg, 259 μmol) and 3-{[1-(pyrimidin-5-yl)ethyl]oxy}-5-(4,4,5,5-tetramethyl-1,3,2-dioxaborolan-2-yl)pyridin-2-amine (115 mg, 336 μmol, stereoisomer 2).

1H-NMR (400 MHz, DMSO-d6): δ [ppm]=1.02 (t, 3H), 1.62 (d, 3H), 1.98-2.11 (m, 2H), 3.00-3.10 (m, 2H), 3.35-3.42 (m, 3H), 3.44-3.52 (m, 1H), 4.15 (t, 2H), 5.79 (q, 1H), 5.96 (s, 2H), 6.15 (t, 1H), 6.30 (s, 1H), 7.42 (d, 1H), 7.91 (d, 1H), 8.98 (s, 2H), 9.11 (s, 1H).

### Example 530

### ((3R)-2′-{6-amino-5-[1-(pyrimidin-5-yl)ethoxy]pyridin-3-yl}-N-ethyl-5′,6′-dihydro-1H-spiro[pyrrolidine-3,4′-pyrrolo[1,2-b]pyrazole]-1-carboxamide (Isomer 2)

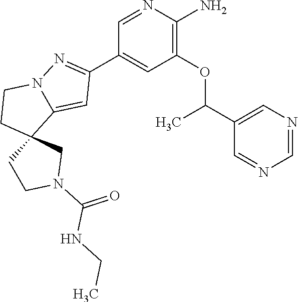 [SMILES: CCNC(=O)N1CC[C@@]2(CCn3nc(-c4cnc(N)c(OC(C)c5cncnc5)c4)cc32)C1]

Example 530 was prepared in analogy to Example 461 using (3R)-1-(ethylcarbamoyl)-5′,6′-dihydrospiro[pyrrolidine-3,4′-pyrrolo[1,2-b]pyrazol]-2′-yl trifluoromethanesulfonate (98.8 mg, 259 μmol) and 3-{[1-(pyrimidin-5-yl)ethyl]oxy}-5-(4,4,5,5-tetramethyl-1,3,2-dioxaborolan-2-yl)pyridin-2-amine (115 mg, 336 μmol, stereoisomer 2).

[α]²⁰_{D}: +125.9° (c=1.00, DMSO)

¹H-NMR (400 MHz, DMSO-d6) δ [ppm]: 0.999 (5.23), 1.017 (11.91), 1.035 (5.29), 1.066 (2.84), 1.613 (7.15), 1.629 (7.23), 2.018 (0.90), 2.032 (1.35), 2.044 (1.83), 2.062 (0.83), 2.518 (4.42), 2.523 (2.11), 3.017 (0.63), 3.034 (2.03), 3.048 (2.25), 3.052 (2.23), 3.066 (2.02), 3.084 (0.60), 3.358 (0.97), 3.383 (4.66), 3.388 (4.14), 3.402 (1.49), 3.413 (0.73), 3.420 (0.60), 3.456 (0.53), 3.474 (0.95), 3.482 (0.57), 3.488 (0.69), 3.499 (0.57), 3.938 (0.53), 4.127 (1.78), 4.145 (3.01), 4.162 (1.78), 5.766 (0.45), 5.782 (1.60), 5.798 (1.61), 5.813 (0.47), 5.962 (4.70), 6.135 (0.83), 6.149 (1.70), 6.163 (0.84), 6.307 (7.98), 7.418 (3.03), 7.423 (3.11), 7.909 (4.15), 7.913 (4.13), 8.986 (16.00), 9.115 (8.60).

### Example 531

### (rac)-2′-[6-amino-5-(difluoromethyl)pyridin-3-yl]-N-ethyl-5′,6′-dihydrospiro[pyrrolidine-3,4′-pyrrolo[1,2-b]pyrazole]-1-carboxamide

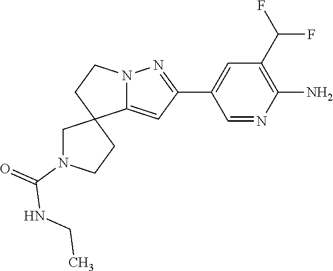 [SMILES: CCNC(=O)N1CCC2(CCn3nc(-c4cnc(N)c(C(F)F)c4)cc32)C1]

Example 531 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (102 mg, 266 μmol) and [6-amino-5-(difluoromethyl)pyridin-3-yl]boronic acid (100 mg, 75% purity, 399 μmol).

LC-MS (method 1): Rₜ=0.80 min; MS (ESIpos): m/z=377 [M+H]⁺

¹H-NMR (400 MHz, DMSO-d6) δ [ppm]: 1.000 (5.76), 1.018 (13.49), 1.036 (6.25), 1.232 (0.60), 2.032 (1.11), 2.045 (1.85), 2.060 (2.54), 2.078 (1.16), 2.090 (0.55), 2.518 (16.00), 2.536 (5.08), 2.673 (1.17), 3.018 (0.74), 3.035 (2.47), 3.050 (2.79), 3.053 (2.93), 3.067 (2.44), 3.085 (0.76), 3.372 (1.88), 3.379 (1.41), 3.400 (9.52), 3.413 (2.09), 3.468 (0.76), 3.485 (1.28), 3.500 (0.97), 3.507 (0.81), 3.525 (0.49), 4.155 (2.29), 4.173 (4.00), 4.190 (2.63), 6.138 (0.95), 6.152 (1.97), 6.165 (1.14), 6.338 (3.80), 6.400 (8.92), 6.433 (1.15), 6.912 (1.29), 7.048 (2.79), 7.184 (1.12), 7.612 (0.59), 7.966 (2.73), 8.358 (0.49), 8.365 (0.54), 8.474 (2.68), 8.477 (2.73), 8.479 (2.71), 8.653 (0.60), 8.658 (0.60), 9.927 (1.23).

### Example 532

### 2′-{6-amino-5-[(1S)-1-(2-chlorophenyl)ethoxy]pyridin-3-yl}-N-ethyl-5′,6′-dihydrospiro[pyrrolidine-3,4′-pyrrolo[1,2-b]pyrazole]-1-carboxamide (Mixture of Stereoisomers)

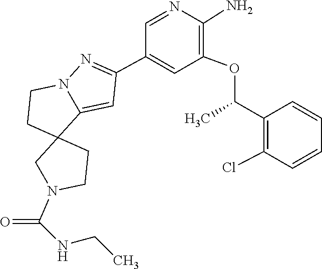 [SMILES: CCNC(=O)N1CCC2(CCn3nc(-c4cnc(N)c(O[C@@H](C)c5ccccc5Cl)c4)cc32)C1]

Example 532 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (40.8 mg, 107 μmol) and 3-[(1S)-1-(2-chlorophenyl)ethoxy]-5-(4,4,5,5-tetramethyl-1,3,2-dioxaborolan-2-yl)pyridin-2-amine (60.0 mg, 160 μmol).

1H-NMR (400 MHz, DMSO-d6): δ [ppm]=7.85 (dd, 1H), 7.59 (dt, 1H), 7.46 (ddd, 1H), 7.39-7.32 (m, 1H), 7.32-7.26 (m, 1H), 7.08 (t, 1H), 6.17 (d, 1H), 6.16-6.12 (m, 1H), 5.94 (s, 2H), 5.81-5.72 (m, 1H), 4.24 (dd, 1H), 4.12 (t, 2H), 3.54-3.42 (m, 1H), 3.37 (d, 2H), 3.10-2.98 (m, 2H), 2.10-1.95 (m, 2H), 1.60 (d, 3H), 1.46-1.31 (m, 2H), 1.02 (td, 3H)

LC-MS (method 1): Rₜ=1.08 min; MS (ESIneg): m/z=479 [M−H]⁻

### Example 533

### 2′-{6-amino-5-[(1R)-1-(2-chlorophenyl)ethoxy]pyridin-3-yl}-N-ethyl-5′,6′-dihydrospiro[pyrrolidine-3,4′-pyrrolo[1,2-b]pyrazole]-1-carboxamide (Mixture of Stereoisomers)

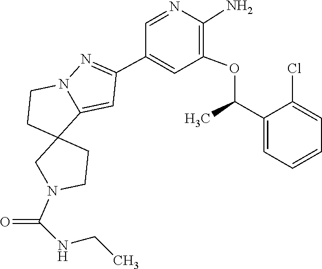 [SMILES: CCNC(=O)N1CCC2(CCn3nc(-c4cnc(N)c(O[C@H](C)c5ccccc5Cl)c4)cc32)C1]

Example 533 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (40.8 mg, 107 μmol) and 3-[(1R)-1-(2-chlorophenyl)ethoxy]-5-(4,4,5,5-tetramethyl-1,3,2-dioxaborolan-2-yl)pyridin-2-amine (60.0 mg, 160 μmol).

1H-NMR (400 MHz, DMSO-d6): δ [ppm]=7.85 (dd, 1H), 7.59 (dt, 1H), 7.46 (ddd, 1H), 7.39-7.32 (m, 1H), 7.32-7.26 (m, 1H), 7.08 (t, 1H), 6.17 (d, 1H), 6.16-6.12 (m, 1H), 5.94 (s, 2H), 5.81-5.72 (m, 1H), 4.24 (dd, 1H), 4.12 (t, 2H), 3.54-3.42 (m, 1H), 3.37 (d, 2H), 3.10-2.98 (m, 2H), 2.10-1.95 (m, 2H), 1.60 (d, 3H), 1.46-1.31 (m, 2H), 1.02 (td, 3H).

### Example 534

### (rac)-2′-{6-amino-5-[(3-methylphenyl)methoxy]pyridin-3-yl}-N-ethyl-5′,6′-dihydrospiro[pyrrolidine-3,4′-pyrrolo[1,2-b]pyrazole]-1-carboxamide

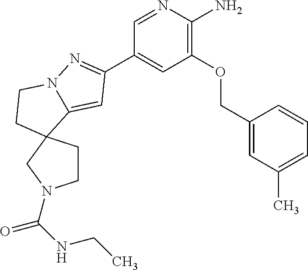 [SMILES: CCNC(=O)N1CCC2(CCn3nc(-c4cnc(N)c(OCc5cccc(C)c5)c4)cc32)C1]

Example 534 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (24.7 mg, 64.6 μmol) and {6-amino-5-[(3-methylphenyl)methoxy]pyridin-3-yl}boronic acid (25.0 mg, 96.9 μmol).

LC-MS (method 1): Rₜ=1.12 min; MS (ESIneg): m/z=445 [M−H]⁻

¹H-NMR (400 MHz, DMSO-d6) δ [ppm]: 1.002 (3.03), 1.020 (7.27), 1.038 (3.20), 1.232 (0.97), 1.528 (16.00), 2.026 (0.51), 2.040 (0.77), 2.053 (1.05), 2.071 (0.45), 2.326 (9.15), 2.518 (4.59), 2.523 (3.21), 2.528 (1.41), 2.540 (0.47), 2.665 (0.73), 2.669 (1.00), 2.673 (0.69), 3.038 (1.19), 3.051 (1.30), 3.055 (1.25), 3.069 (1.16), 3.164 (2.14), 3.397 (5.21), 3.424 (1.03), 3.465 (0.53), 3.483 (0.70), 3.497 (0.51), 3.505 (0.44), 4.139 (1.05), 4.157 (1.68), 4.174 (0.99), 5.133 (4.01), 5.778 (2.49), 6.152 (0.46), 6.166 (0.92), 6.179 (0.43), 6.335 (4.72), 7.128 (0.68), 7.144 (0.74), 7.255 (0.43), 7.274 (1.48), 7.291 (2.52), 7.338 (1.44), 7.418 (1.80), 7.422 (1.77), 7.931 (2.62), 7.936 (2.53).

### Example 535

### (rac)-2′-{6-amino-5-[(1-methyl-1H-pyrazol-5-yl)methoxy]pyridin-3-yl}-N-ethyl-5′,6′-dihydrospiro[pyrrolidine-3,4′-pyrrolo[1,2-b]pyrazole]-1-carboxamide

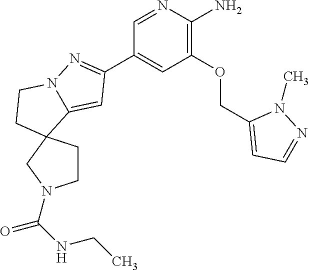 [SMILES: CCNC(=O)N1CCC2(CCn3nc(-c4cnc(N)c(OCc5ccnn5C)c4)cc32)C1]

Example 535 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (108 mg, 283 μmol) and 3-[(1-methyl-1H-pyrazol-5-yl)methoxy]-5-(4,4,5,5-tetramethyl-1,3,2-dioxaborolan-2-yl)pyridin-2-amine (200 mg, 70% purity, 424 μmol).

LC-MS (method 1): Rₜ=0.75 min; MS (ESIpos): m/z=437.5 [M+H]⁺

¹H-NMR (400 MHz, DMSO-d6) δ [ppm]: 1.003 (0.32), 1.021 (0.71), 1.039 (0.32), 1.232 (0.15), 1.313 (0.11), 1.328 (0.10), 2.051 (0.08), 2.064 (0.10), 2.331 (0.19), 2.337 (0.09), 2.518 (1.08), 2.523 (0.82), 2.540 (16.00), 2.673 (0.19), 2.678 (0.09), 3.039 (0.13), 3.053 (0.13), 3.057 (0.13), 3.070 (0.12), 3.407 (0.40), 3.825 (0.10), 3.870 (1.48), 3.882 (0.10), 4.153 (0.11), 4.171 (0.17), 4.188 (0.10), 5.227 (0.45), 5.787 (0.26), 6.159 (0.10), 6.364 (0.49), 6.426 (0.25), 6.431 (0.24), 7.376 (0.25), 7.381 (0.26), 7.507 (0.18), 7.512 (0.18), 7.955 (0.25), 7.959 (0.24).

### Example 536

### (rac)-2′-{6-amino-5-[(1-methyl-1H-pyrazol-4-yl)methoxy]pyridin-3-yl}-N-ethyl-5′,6′-dihydrospiro[pyrrolidine-3,4′-pyrrolo[1,2-b]pyrazole]-1-carboxamide

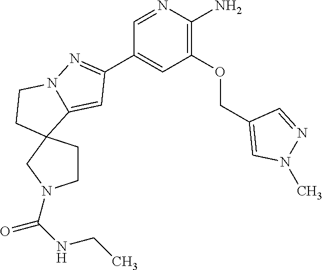 [SMILES: CCNC(=O)N1CCC2(CCn3nc(-c4cnc(N)c(OCc5cnn(C)c5)c4)cc32)C1]

Example 536 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (38.6 mg, 101 μmol) and 3-[(1-methyl-1H-pyrazol-4-yl)methoxy]-5-(4,4,5,5-tetramethyl-1,3,2-dioxaborolan-2-yl)pyridin-2-amine (50.0 mg, 151 μmol).

LC-MS (method 1): Rₜ=0.73 min; MS (ESIpos): m/z=437 [M+H]⁺

¹H-NMR (400 MHz, DMSO-d6) δ [ppm]: 1.003 (3.67), 1.021 (8.95), 1.039 (3.91), 1.065 (0.61), 1.154 (1.30), 1.172 (2.38), 1.190 (1.17), 1.231 (0.51), 1.600 (2.84), 1.987 (4.60), 2.031 (0.60), 2.045 (0.93), 2.060 (1.24), 2.078 (0.53), 2.518 (2.92), 2.523 (1.39), 2.534 (1.35), 2.540 (2.31), 2.994 (0.56), 3.021 (0.45), 3.039 (1.41), 3.053 (1.57), 3.057 (1.50), 3.070 (1.40), 3.088 (0.42), 3.165 (0.63), 3.404 (5.68), 3.428 (0.85), 3.472 (0.50), 3.490 (0.73), 3.497 (0.48), 3.504 (0.53), 3.512 (0.44), 3.516 (0.44), 3.821 (16.00), 4.017 (1.03), 4.035 (1.02), 4.148 (1.27), 4.165 (2.06), 4.182 (1.21), 5.004 (5.35), 5.673 (2.97), 6.152 (0.57), 6.166 (1.14), 6.179 (0.56), 6.363 (5.61), 7.440 (2.16), 7.445 (2.18), 7.531 (3.53), 7.533 (3.64), 7.825 (3.35), 7.925 (3.29), 7.929 (3.23).

### Example 537

### (rac)-2′-{6-amino-5-[(1,5-dimethyl-1H-pyrazol-3-yl)methoxy]pyridin-3-yl}-N-ethyl-5′,6′-dihydrospiro[pyrrolidine-3,4′-pyrrolo[1,2-b]pyrazole]-1-carboxamide

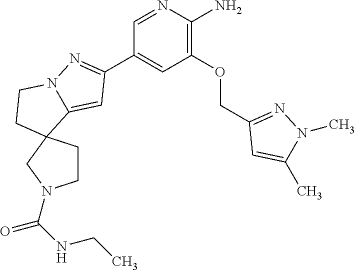 [SMILES: CCNC(=O)N1CCC2(CCn3nc(-c4cnc(N)c(OCc5cc(C)n(C)n5)c4)cc32)C1]

Example 537 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (68.1 mg, 178 μmol) and {6-amino-5-[(1,5-dimethyl-1H-pyrazol-3-yl)methoxy]pyridin-3-yl}boronic acid (70.0 mg, 267 μmol).

LC-MS (method 1): Rₜ=0.80 min; MS (ESIpos): m/z=451 [M+H]⁺

¹H-NMR (400 MHz, DMSO-d6) δ [ppm]: 1.003 (3.26), 1.021 (7.83), 1.038 (3.48), 2.033 (0.56), 2.040 (0.58), 2.049 (0.86), 2.058 (1.09), 2.065 (0.63), 2.076 (0.49), 2.227 (10.35), 2.518 (2.63), 2.523 (2.79), 2.539 (1.46), 3.020 (0.40), 3.038 (1.27), 3.052 (1.42), 3.056 (1.35), 3.070 (1.24), 3.390 (1.08), 3.403 (4.72), 3.415 (1.14), 3.433 (0.45), 3.483 (0.66), 3.497 (0.44), 3.693 (2.78), 3.696 (16.00), 4.154 (1.12), 4.172 (1.79), 4.189 (1.08), 5.000 (0.59), 5.024 (4.74), 5.999 (0.40), 6.156 (3.27), 6.175 (0.52), 6.366 (4.62), 7.541 (1.74), 7.546 (1.75), 7.911 (2.68), 7.915 (2.53).

### Example 538

### (rac)-2′-{6-amino-5-[(4-methylphenyl)methoxy]pyridin-3-yl}-N-ethyl-5′,6′-dihydrospiro[pyrrolidine-3,4′-pyrrolo[1,2-b]pyrazole]-1-carboxamide

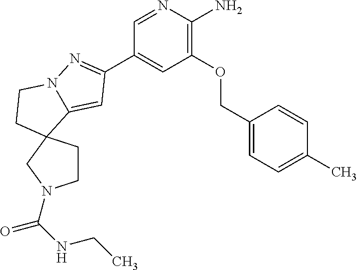 [SMILES: CCNC(=O)N1CCC2(CCn3nc(-c4cnc(N)c(OCc5ccc(C)cc5)c4)cc32)C1]

Example 538 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (49.4 mg, 129 μmol) and {6-amino-5-[(4-methylphenyl)methoxy]pyridin-3-yl}boronic acid (50.0 mg, 194 μmol).

LC-MS (method 1): Rₜ=1.01 min; MS (ESIpos): m/z=447.5 [M+H]⁺

¹H-NMR (400 MHz, DMSO-d6) δ [ppm]: 1.003 (6.34), 1.021 (14.07), 1.038 (6.44), 2.024 (1.24), 2.039 (1.96), 2.052 (2.52), 2.070 (1.09), 2.302 (16.00), 2.523 (2.50), 2.527 (2.91), 2.539 (8.33), 3.020 (0.86), 3.038 (2.69), 3.053 (3.06), 3.056 (3.01), 3.071 (2.63), 3.088 (0.79), 3.371 (1.28), 3.380 (1.87), 3.397 (9.63), 3.423 (1.08), 3.465 (0.76), 3.483 (1.31), 3.490 (0.84), 3.497 (0.96), 3.508 (0.79), 4.138 (2.46), 4.155 (4.12), 4.173 (2.36), 5.131 (8.71), 5.784 (3.44), 6.142 (1.15), 6.156 (2.29), 6.169 (1.14), 6.337 (8.95), 7.188 (4.44), 7.207 (5.38), 7.385 (5.91), 7.404 (4.91), 7.418 (4.21), 7.423 (4.31), 7.924 (5.19), 7.928 (5.13).

### Example 539

### 2′-{6-amino-5-[(1R)-1-(3-methylphenyl)ethoxy]pyridin-3-yl}-N-ethyl-5′,6′-dihydrospiro[pyrrolidine-3,4′-pyrrolo[1,2-b]pyrazole]-1-carboxamide (Mixture of Stereoisomers)

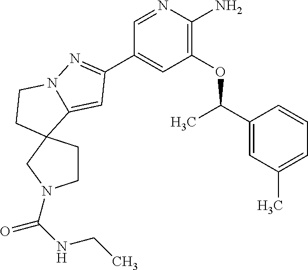 [SMILES: CCNC(=O)N1CCC2(CCn3nc(-c4cnc(N)c(O[C@H](C)c5cccc(C)c5)c4)cc32)C1]

Example 539 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (65.6 mg, 171 μmol) and {6-amino-5-[(1R)-1-(3-methylphenyl)ethoxy]pyridin-3-yl}boronic acid (70.0 mg, 257 μmol).

LC-MS (method 1): Rₜ=1.04 min; MS (ESIpos): m/z=461 [M+H]⁺

¹H-NMR (400 MHz, DMSO-d6) δ [ppm]: 0.997 (4.29), 1.001 (4.38), 1.015 (9.70), 1.019 (9.76), 1.033 (4.51), 1.037 (4.48), 1.066 (5.58), 1.233 (0.72), 1.545 (9.96), 1.561 (9.95), 2.028 (2.41), 2.073 (0.64), 2.283 (16.00), 2.465 (3.14), 2.518 (8.35), 2.523 (5.65), 2.539 (0.83), 3.018 (0.67), 3.032 (2.37), 3.050 (3.43), 3.067 (2.24), 3.081 (0.61), 3.294 (0.45), 3.304 (1.18), 3.372 (9.11), 3.444 (0.81), 3.463 (1.33), 3.476 (1.04), 3.502 (0.49), 3.941 (0.92), 4.105 (2.76), 4.123 (4.75), 4.140 (2.67), 5.500 (0.66), 5.515 (2.33), 5.531 (2.31), 5.547 (0.68), 5.817 (6.84), 6.131 (1.23), 6.145 (2.41), 6.159 (1.19), 6.217 (6.22), 6.224 (5.85), 7.048 (1.72), 7.064 (2.03), 7.198 (1.04), 7.217 (3.21), 7.234 (8.13), 7.255 (0.89), 7.284 (3.89), 7.840 (3.57), 7.844 (5.09), 7.848 (3.56).

### Example 540

### (rac)-2′-{6-amino-5-[(propan-2-yl)oxy]pyridin-3-yl}-N-ethyl-5′,6′-dihydrospiro[pyrrolidine-3,4′-pyrrolo[1,2-b]pyrazole]-1-carboxamide

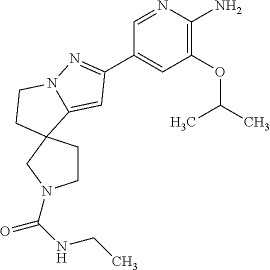 [SMILES: CCNC(=O)N1CCC2(CCn3nc(-c4cnc(N)c(OC(C)C)c4)cc32)C1]

Example 540 was prepared in analogy to Example 461 using (rac)-1-(ethylcarbamoyl)-5′,6′-dihydrospiro[pyrrolidine-3,4′-pyrrolo[1,2-b]pyrazol]-2′-yl trifluoromethanesulfonate (75.0 mg, 196 μmol) and 3-[(propan-2-yl)oxy]-5-(4,4,5,5-tetramethyl-1,3,2-dioxaborolan-2-yl)pyridin-2-amine (69.9 mg, 78% purity, 196 μmol).

¹H-NMR (400 MHz, DMSO-d6): δ [ppm]=1.02 (t, 3H), 1.29 (d, 6H), 1.99-2.11 (m, 2H), 2.52-2.55 (m, 2H), 3.00-3.10 (m, 2H), 3.40 (s, 3H), 3.45-3.55 (m, 1H), 4.16 (t, 2H), 4.61 (dt, 1H), 5.65 (s, 2H), 6.15 (t, 1H), 6.34 (s, 1H), 7.33 (d, 1H), 7.90 (d, 1H).

LC-MS (method 1): Rₜ=0.86 min; MS (ESIpos): m/z=385 [M+H]⁺

### Example 541

### (3R)-2′-{6-amino-5-[(1S)-1-(1-methyl-1H-pyrazol-3-yl)ethoxy]pyridin-3-yl}-N-ethyl-5′,6′-dihydrospiro[pyrrolidine-3,4′-pyrrolo[1,2-b]pyrazole]-1-carboxamide

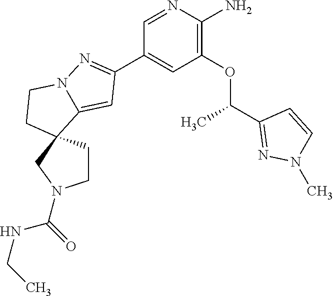 [SMILES: CCNC(=O)N1CC[C@@]2(CCn3nc(-c4cnc(N)c(O[C@@H](C)c5ccn(C)n5)c4)cc32)C1]

Example 541 was prepared in analogy to Example 461 using (3R)-1-(ethylcarbamoyl)-5′,6′-dihydrospiro[pyrrolidine-3,4′-pyrrolo[1,2-b]pyrazol]-2′-yl trifluoromethanesulfonate (65.7 mg, 172 μmol) and {6-amino-5-[(1S)-1-(1-methyl-1H-pyrazol-3-yl)ethoxy]pyridin-3-yl}boronic acid (225 mg, 20% purity, 172 μmol).

LC-MS (method 1): Rt=0.78 min; MS (ESIpos): m/z=452 [M+H]⁺

1H-NMR (400 MHz, CHLOROFORM-d): δ [ppm]=1.16 (t, 3H), 1.71 (d, 3H), 2.01-2.10 (m, 1H), 2.22 (dt, 1H), 2.48-2.57 (m, 1H), 2.58-2.68 (m, 1H), 3.30 (qd, 2H), 3.47-3.57 (m, 3H), 3.58-3.67 (m, 1H), 3.86 (s, 3H), 4.23 (t, 3H), 4.78 (s, 2H), 5.50 (d, 1H), 6.12 (s, 1H), 6.22 (d, 1H), 7.49 (d, 1H), 8.02 (d, 1H).

[α]²⁰_{D}: −39.29° (c=1.00, MeOH)

### Example 542

### (3R)-2′-{6-amino-5-[(1R)-1-(1-methyl-1H-pyrazol-3-yl)ethoxy]pyridin-3-yl}-N-ethyl-5′,6′-dihydrospiro[pyrrolidine-3,4′-pyrrolo[1,2-b]pyrazole]-1-carboxamide

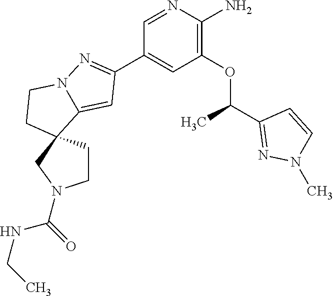 [SMILES: CCNC(=O)N1CC[C@@]2(CCn3nc(-c4cnc(N)c(O[C@H](C)c5ccn(C)n5)c4)cc32)C1]

Example 542 was prepared in analogy to Example 461 using (3R)-1-(ethylcarbamoyl)-5′,6′-dihydrospiro[pyrrolidine-3,4′-pyrrolo[1,2-b]pyrazol]-2′-yl trifluoromethanesulfonate (62.2 mg, 163 μmol) and {6-amino-5-[(1R)-1-(1-methyl-1H-pyrazol-3-yl)ethoxy]pyridin-3-yl}boronic acid (213 mg, 20% purity, 163 μmol).

LC-MS (method 1): Rt=0.78 min; MS (ESIpos): m/z=452 [M+H]⁺

1H-NMR (400 MHz, CHLOROFORM-d): δ [ppm]=1.16 (t, 3H), 1.71 (d, 3H), 2.01-2.10 (m, 1H), 2.22 (dt, 1H), 2.48-2.57 (m, 1H), 2.58-2.68 (m, 1H), 3.30 (qd, 2H), 3.47-3.57 (m, 3H), 3.58-3.67 (m, 1H), 3.86 (s, 3H), 4.23 (t, 3H), 4.78 (s, 2H), 5.50 (d, 1H), 6.12 (s, 1H), 6.22 (d, 1H), 7.49 (d, 1H), 8.02 (d, 1H).

[α]²⁰_{D}: +145.6° (c=1.00, MeOH)

### Example 543

### (3R)-2′-(6-amino-5-{[1-(3,5-difluoropyridin-2-yl)ethyl]oxy}pyridin-3-yl)-N-ethyl-5′,6′-dihydrospiro[pyrrolidine-3,4′-pyrrolo[1,2-b]pyrazole]-1-carboxamide (Stereoisomer 1)

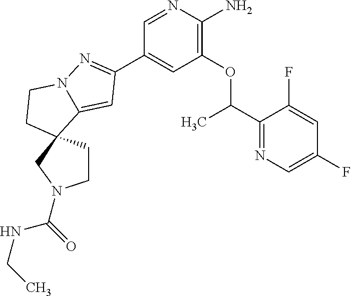 [SMILES: CCNC(=O)N1CC[C@@]2(CCn3nc(-c4cnc(N)c(OC(C)c5ncc(F)cc5F)c4)cc32)C1]

Example 543 was prepared in analogy to Example 461 using (3R)-1-(ethylcarbamoyl)-5′,6′-dihydrospiro[pyrrolidine-3,4′-pyrrolo[1,2-b]pyrazol]-2′-yl trifluoromethanesulfonate (75.0 mg, 196 μmol) and (6-amino-5-{[1-(3,5-difluoropyridin-2-yl)ethyl]oxy}pyridin-3-yl)boronic acid (223 mg, 35% purity, 265 μmol, stereoisomer 1).

1H-NMR (400 MHz, CHLOROFORM-d): δ [ppm]=1.17 (t, 3H), 1.76 (d, 3H), 2.03-2.12 (m, 1H), 2.23 (dt, 1H), 2.49-2.59 (m, 1H), 2.59-2.69 (m, 1H), 3.32 (qd, 2H), 3.47-3.59 (m, 3H), 3.59-3.68 (m, 1H), 4.12-4.21 (m, 1H), 4.25 (t, 2H), 4.80 (s, 2H), 5.78 (d, 1H), 6.11 (s, 1H), 7.15-7.23 (m, 1H), 7.43 (d, 1H), 8.03 (d, 1H), 8.34 (d, 1H).

[α]²⁰_{D}: −71.63° (c=1.00, MeOH)

LC-MS (method 1): Rₜ=0.90 min; MS (ESIneg): m/z=482 [M−H]⁻

### Example 544

### (3R)-2′-(6-amino-5-{[1-(3,5-difluoropyridin-2-yl)ethyl]oxy}pyridin-3-yl)-N-ethyl-5′,6′-dihydrospiro[pyrrolidine-3,4′-pyrrolo[1,2-b]pyrazole]-1-carboxamide (Stereoisomer 2)

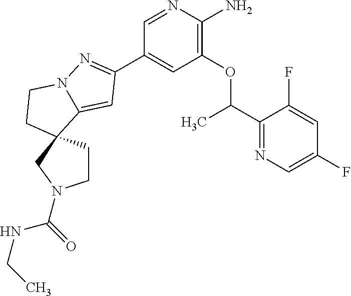 [SMILES: CCNC(=O)N1CC[C@@]2(CCn3nc(-c4cnc(N)c(OC(C)c5ncc(F)cc5F)c4)cc32)C1]

Example 544 was prepared in analogy to Example 461 using (3R)-1-(ethylcarbamoyl)-5′,6′-dihydrospiro[pyrrolidine-3,4′-pyrrolo[1,2-b]pyrazol]-2′-yl trifluoromethanesulfonate (75.0 mg, 196 μmol) and (6-amino-5-{[1-(3,5-difluoropyridin-2-yl)ethyl]oxy}pyridin-3-yl)boronic acid (231 mg, 35% purity, 275 μmol, stereoisomer 2).

LC-MS (method 1): Rt=0.89 min; MS (ESIpos): m/z=485 [M+H]⁺

1H-NMR (400 MHz, CHLOROFORM-d): δ [ppm]=1.17 (t, 3H), 1.76 (d, 3H), 2.08 (s, 1H), 2.19-2.29 (m, 1H), 2.50-2.59 (m, 1H), 2.59-2.69 (m, 1H), 3.31 (dd, 2H), 3.47-3.59 (m, 3H), 3.59-3.68 (m, 1H), 4.12-4.20 (m, 1H), 4.25 (t, 2H), 4.80 (s, 2H), 5.78 (d, 1H), 6.11 (s, 1H), 7.19 (br d, 1H), 7.44 (d, 1H), 8.02 (d, 1H), 8.34 (d, 1H).

[α]²⁰_{D}: +162.73° (c=1.00, MeOH)

### Example 545

### (3R)-2′-{6-amino-5-[(1R)-1-(2-fluorophenyl)ethoxy]pyridin-3-yl}-N-ethyl-5′,6′-dihydrospiro[pyrrolidine-3,4′-pyrrolo[1,2-b]pyrazole]-1-carboxamide

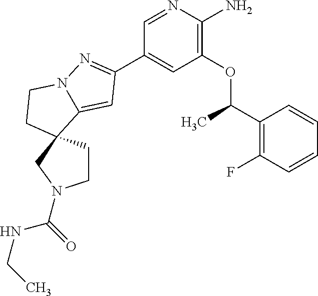 [SMILES: CCNC(=O)N1CC[C@@]2(CCn3nc(-c4cnc(N)c(O[C@H](C)c5ccccc5F)c4)cc32)C1]

Example 545 was prepared in analogy to Example 461 using (3R)-1-(ethylcarbamoyl)-5′,6′-dihydrospiro[pyrrolidine-3,4′-pyrrolo[1,2-b]pyrazol]-2′-yl trifluoromethanesulfonate (75.0 mg, 196 μmol) and 3-[(1R)-1-(2-fluorophenyl)ethoxy]-5-(4,4,5,5-tetramethyl-1,3,2-dioxaborolan-2-yl)pyridin-2-amine (229 mg, 43% purity, 275 μmol).

LC-MS (method 1): Rt=1.00 min; MS (ESIpos): m/z=466 [M+H]⁺

1H-NMR (400 MHz, CHLOROFORM-d): δ [ppm]=1.16 (t, 3H), 1.69 (d, 3H), 2.01-2.11 (m, 1H), 2.21 (s, 1H), 2.48-2.57 (m, 1H), 2.57-2.67 (m, 1H), 3.31 (dd, 2H), 3.46-3.57 (m, 3H), 3.58-3.67 (m, 1H), 4.15 (s, 1H), 4.23 (t, 2H), 4.77 (s, 2H), 5.76 (q, 1H), 6.07 (s, 1H), 7.01-7.16 (m, 2H), 7.21-7.25 (m, 1H), 7.27-7.29 (m, 1H), 7.41 (d, 1H), 8.01 (d, 1H).

[α]²⁰_{D}: +156.09° (c=1.00, MeOH)

### Example 546

### (3R)-2′-{6-amino-5-[(1S)-1-(2-fluorophenyl)ethoxy]pyridin-3-yl}-N-ethyl-5′,6′-dihydrospiro[pyrrolidine-3,4′-pyrrolo[1,2-b]pyrazole]-1-carboxamide

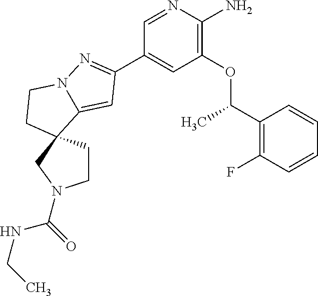 [SMILES: CCNC(=O)N1CC[C@@]2(CCn3nc(-c4cnc(N)c(O[C@@H](C)c5ccccc5F)c4)cc32)C1]

Example 546 was prepared in analogy to Example 461 using (3R)-1-(ethylcarbamoyl)-5′,6′-dihydrospiro[pyrrolidine-3,4′-pyrrolo[1,2-b]pyrazol]-2′-yl trifluoromethanesulfonate (75.0 mg, 196 μmol) and 3-[(1S)-1-(2-fluorophenyl)ethoxy]-5-(4,4,5,5-tetramethyl-1,3,2-dioxaborolan-2-yl)pyridin-2-amine (219 mg, 45% purity, 275 μmol).

LC-MS (method 1): Rt=1.00 min; MS (ESIpos): m/z=466 [M+H]⁺

1H-NMR (400 MHz, CHLOROFORM-d): δ [ppm]=1.17 (t, 3H), 1.69 (d, 3H), 2.00-2.10 (m, 1H), 2.17-2.28 (m, 1H), 2.54 (s, 1H), 2.60 (s, 1H), 3.26-3.37 (m, 2H), 3.46-3.57 (m, 3H), 3.58-3.67 (m, 1H), 4.12-4.19 (m, 1H), 4.23 (t, 2H), 4.77 (s, 2H), 5.76 (d, 1H), 6.07 (s, 1H), 7.11 (d, 2H), 7.21-7.25 (m, 1H), 7.27 (s, 1H), 7.41 (d, 1H), 8.01 (d, 1H).

[α]²⁰_{D}: −44.34° (c=1.00, MeOH)

### Example 547

### (rac)-2′-(5-acetyl-6-aminopyridin-3-yl)-N-(1-phenylcyclobutyl)-5′,6′-dihydrospiro[pyrrolidine-3,4′-pyrrolo[1,2-b]pyrazole]-1-carboxamide

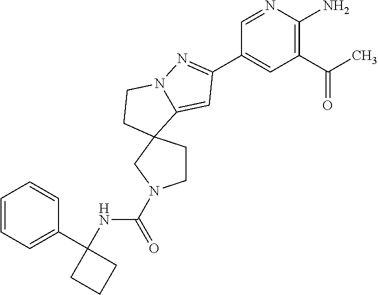 [SMILES: CC(=O)c1cc(-c2cc3n(n2)CCC32CCN(C(=O)NC3(c4ccccc4)CCC3)C2)cnc1N]

(rac)-1-[(1-phenylcyclobutyl)carbamoyl]-5′,6′-dihydrospiro[pyrrolidine-3,4′-pyrrolo[1,2-b]pyrazol]-2′-yl trifluoromethanesulfonate (194 mg, 400 μmol), (5-acetyl-6-aminopyridin-3-yl)boronic acid (82.8 mg, 460 μmol), XPhos Pd G2 (47.2 mg, 60.0 μmol) and potassium phosphate (2.4 mL, 0.50 M, 1.2 mmol) were solubilised in dioxane (4.0 mL) and the mixture was stirred for 1 h at 100° C. The mixture was diluted with water and extracted three times with ethyl acetate. The combined organic layers were dried and evaporated. The residue was purified by preparative HPLC to give 27.5 mg (97% purity, 14% yield) of the title compound.

1H-NMR (400 MHz, DMSO-d6) δ [ppm]: 1.036 (1.47), 1.053 (2.85), 1.071 (1.56), 1.764 (0.45), 1.769 (0.47), 1.786 (0.49), 1.791 (0.56), 1.982 (0.53), 1.997 (0.41), 2.005 (0.50), 2.009 (0.51), 2.026 (0.41), 2.032 (0.44), 2.045 (0.71), 2.059 (1.09), 2.072 (1.24), 2.089 (0.56), 2.359 (0.40), 2.382 (0.86), 2.390 (1.00), 2.397 (0.83), 2.405 (1.22), 2.411 (0.97), 2.427 (0.71), 2.452 (0.70), 2.475 (1.30), 2.518 (2.01), 2.523 (1.96), 2.537 (1.90), 2.542 (1.85), 2.558 (1.18), 2.596 (16.00), 3.160 (3.62), 3.172 (3.72), 3.418 (0.85), 3.423 (1.03), 3.447 (3.80), 3.535 (0.65), 3.551 (0.49), 3.560 (0.45), 4.101 (0.67), 4.114 (0.66), 4.181 (1.46), 4.198 (2.81), 4.215 (1.43), 6.463 (4.93), 6.704 (2.38), 7.134 (0.71), 7.137 (0.43), 7.152 (1.77), 7.167 (0.72), 7.170 (1.19), 7.173 (0.69), 7.262 (1.98), 7.282 (3.33), 7.296 (0.73), 7.301 (1.92), 7.421 (2.89), 7.423 (3.12), 7.441 (2.60), 7.445 (2.01), 8.381 (2.64), 8.387 (2.78), 8.597

### Example 548

### (3R)-2′-[6-amino-5-(trifluoromethoxy)pyridin-3-yl]-N-[2-(pyridin-2-yl)propan-2-yl]-5′,6′-dihydrospiro[pyrrolidine-3,4′-pyrrolo[1,2-b]pyrazole]-1-carboxamide

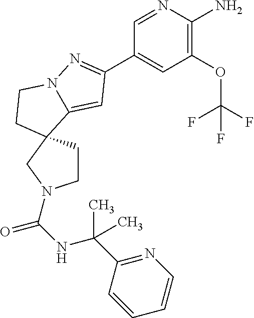 [SMILES: CC(C)(NC(=O)N1CC[C@@]2(CCn3nc(-c4cnc(N)c(OC(F)(F)F)c4)cc32)C1)c1ccccn1]

To a solution of 5-(4,4,5,5-tetramethyl-1,3,2-dioxaborolan-2-yl)-3-(trifluoromethoxy)pyridin-2-amine (116 mg, 382 μmol) in Dioxane (4 mL) under Argon, (3R)-1-{[2-(pyridin-2-yl)propan-2-yl]carbamoyl}-5′,6′-dihydrospiro[pyrrolidine-3,4′-pyrrolo[1,2-b]pyrazol]-2′-yl trifluoromethanesulfonate (190 mg, 401 μmol) and Potassium phosphate (2.3 ml, 0.50 M in water) were added. The mixture was degassed and XPhos Pd G2 (15.0 mg, 19.1 μmol) was added. The reaction mixture was stirred at 100° C. under Argon for 1 h. The mixture was diluted with water and then extracted with Ethyl acetate 2×. The combined organics were washed with Brine, dried over Sodium sulfate, filtered and concentrated. The crude material was purified by basic to give 41.0 mg (99% purity, 21% yield) of the title compound.

1H NMR (DMSO-d6, 400 MHz) δ 8.4-8.5 (m, 1H), 8.34 (d, 1H), 7.76 (t, 1H), 7.7-7.7 (m, 1H), 7.4-7.5 (m, 1H), 7.18 (ddd, 1H), 6.51 (s, 2H), 6.4-6.5 (m, 2H), 4.19 (t, 2H), 3.5-3.6 (m, 1H), 3.4-3.5 (m, 3H), 2.55 (t, 2H), 2.0-2.2 (m, 2H), 1.59 (s, 6H)

LC-MS (method 1): Rₜ=1.02 min; MS (ESIpos): m/z=503 [M+H]⁺

### Example 549

### (3′R)-2-[6-amino-5-(trifluoromethyl)pyridin-3-yl]-N-[2-(pyridin-2-yl)propan-2-yl]-6,7-dihydrospiro[pyrazolo[5,1-c][1,4]oxazine-4,3′-pyrrolidine]-1′-carboxamide

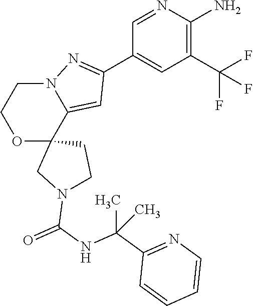 [SMILES: CC(C)(NC(=O)N1CC[C@]2(C1)OCCn1nc(-c3cnc(N)c(C(F)(F)F)c3)cc12)c1ccccn1]

To a solution of 5-(4,4,5,5-tetramethyl-1,3,2-dioxaborolan-2-yl)-3-(trifluoromethyl)pyridin-2-amine (104 mg, 362 μmol) in Dioxane (4 mL), (3′R)-2-bromo-N-[2-(pyridin-2-yl)propan-2-yl]-6,7-dihydrospiro[pyrazolo[5,1-c][1,4]oxazine-4,3′-pyrrolidine]-1′-carboxamide (190 mg, 452 μmol) and Potassium phosphate (2.2 ml, 0.50 M in water) were added. The mixture was degassed and XPhos Pd G2 (14.2 mg, 18.1 μmol) was added. The resultant mixture was stirred at 100° C. under Argon for 1 h. The mixture was diluted with water and then extracted with Ethyl acetate 2×. The combined organics were washed with Brine, dried over Sodium sulfate, filtered and concentrated in vacuo. The crude was purified by basic HPLC to give 49.0 mg (99% purity, 27% yield) of the title compound.

1H NMR (DMSO-d6, 400 MHz) δ 8.59 (d, 1H), 8.4-8.5 (m, 1H), 8.02 (d, 1H), 7.72 (dt, 1H), 7.4-7.5 (m, 1H), 7.19 (ddd, 1H), 6.78 (s, 1H), 6.60 (s, 2H), 6.43 (s, 1H), 4.1-4.2 (m, 4H), 3.82 (d, 1H), 3.63 (br t, 1H), 3.52 (br d, 1H), 3.4-3.5 (m, 1H), 2.2-2.4 (m, 2H), 1.58 (d, 6H)

LC-MS (method 1): Rₜ=1.01 min; MS (ESIpos): m/z=503 [M+H]⁺

### Example 550

### (3R)-2′-(6-amino-5-cyanopyridin-3-yl)-N-[2-(pyridin-2-yl)propan-2-yl]-5′,6′-dihydrospiro[pyrrolidine-3,4′-pyrrolo[1,2-b]pyrazole]-1-carboxamide

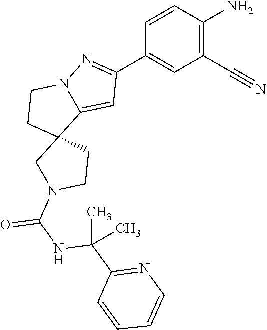 [SMILES: CC(C)(NC(=O)N1CC[C@@]2(CCn3nc(-c4ccc(N)c(C#N)c4)cc32)C1)c1ccccn1]

To a solution of 2-amino-5-(4,4,5,5-tetramethyl-1,3,2-dioxaborolan-2-yl)pyridine-3-carbonitrile (78.7 mg, 321 μmol) in Dioxane (3.5 mL), (3R)-1-{[2-(pyridin-2-yl)propan-2-yl]carbamoyl}-5′,6′-dihydrospiro[pyrrolidine-3,4′-pyrrolo[1,2-b]pyrazol]-2′-yl trifluoromethanesulfonate (190 mg, 401 μmol) and Potassium phosphate (1.9 ml, 0.50 M in water) were added. The mixture was degassed and XPhos Pd G2 (12.6 mg, 16.1 μmol) was added. The resultant mixture was stirred at 100° C. under Argon for 1 h. The mixture was diluted with water and then extracted with Ethyl acetate 2×. The combined organics were washed with Brine, dried over Sodium sulfate, filtered and concentrated in vacuo. The crude was purified by basic HPLC to give 45.0 mg (99% purity, 31% yield) of the title compound.

1H NMR (DMSO-d6, 400 MHz) δ 8.62 (d, 1H), 8.4-8.5 (m, 1H), 8.15 (d, 1H), 7.71 (dt, 1H), 7.47 (d, 1H), 7.19 (ddd, 1H), 7.00 (s, 2H), 6.47 (s, 1H), 6.43 (s, 1H), 4.20 (t, 2H), 3.5-3.6 (m, 1H), 3.4-3.5 (m, 3H), 2.56 (t, 2H), 2.0-2.1 (m, 2H), 1.59 (s, 6H)

LC-MS (method 1): Rₜ=0.89 min; MS (ESIpos): m/z=444 [M+H]⁺

### Example 551

### (3′R)-2-(6-amino-5-cyanopyridin-3-yl)-N-[2-(pyridin-2-yl)propan-2-yl]-6,7-dihydrospiro[pyrazolo[5,1-c][1,4]oxazine-4,3′-pyrrolidine]-1′-carboxamide

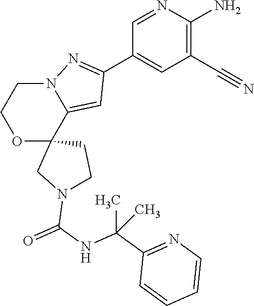 [SMILES: CC(C)(NC(=O)N1CC[C@]2(C1)OCCn1nc(-c3cnc(N)c(C#N)c3)cc12)c1ccccn1]

To a solution of 2-amino-5-(4,4,5,5-tetramethyl-1,3,2-dioxaborolan-2-yl)pyridine-3-carbonitrile (88.6 mg, 362 μmol) in Dioxane (4 mL), (3′R)-2-bromo-N-[2-(pyridin-2-yl)propan-2-yl]-6,7-dihydrospiro[pyrazolo[5,1-c][1,4]oxazine-4,3′-pyrrolidine]-1′-carboxamide (190 mg, 452 μmol) and Potassium phosphate (2.2 ml, 0.50 M in water) were added. The mixture was degassed and XPhos Pd G2 (14.2 mg, 18.1 μmol) was added. The resultant mixture was stirred at 100° C. under Argon for 1 h. The mixture was diluted with water and then extracted with Ethyl acetate 2×. The combined organics were washed with Brine, dried over Sodium sulfate, filtered and concentrated in vacuo. The crude was purified by acidic HPLC. The compound was basified by dissolved in Ethyl acetate and washed with std. NaHCO3-solution. The organics were dried over sodium sulfate, filtered and concentrated to give 57.0 mg (99% purity, 34% yield) of the title compound.

1H NMR (DMSO-d6, 400 MHz) δ 8.61 (d, 1H), 8.4-8.5 (m, 1H), 8.13 (d, 1H), 7.72 (dt, 1H), 7.4-7.5 (m, 1H), 7.19 (ddd, 1H), 7.05 (s, 2H), 6.73 (s, 1H), 6.43 (s, 1H), 4.1-4.2 (m, 4H), 3.81 (br d, 1H), 3.63 (br t, 1H), 3.4-3.5 (m, 2H), 2.3-2.4 (m, 1H), 2.1-2.3 (m, 1H), 1.58 (d, 6H)

LC-MS (method 1): Rₜ=0.88 min; MS (ESIpos): m/z=460 [M+H]⁺

### Example 552

### (rac)-2′-[6-amino-5-(phenylcarbamoyl)pyridin-3-yl]-N-ethyl-5′,6′-dihydrospiro[pyrrolidine-3,4′-pyrrolo[1,2-b]pyrazole]-1-carboxamide

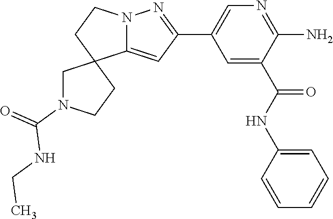 [SMILES: CCNC(=O)N1CCC2(CCn3nc(-c4cnc(N)c(C(=O)Nc5ccccc5)c4)cc32)C1]

2-amino-5-[(rac)-1-(ethylcarbamoyl)-5′,6′-dihydrospiro[pyrrolidine-3,4′-pyrrolo[1,2-b]pyrazol]-2′-yl]pyridine-3-carboxylic acid (50.0 mg, 135 μmol) was solubilised in THF (1000 μL), aniline (15.1 mg, 162 μmol), HATU (77.0 mg, 202 μmol) and triethylamine (47 μL, 340 μmol) were added and the mixture was stirred for 6 h at rt. aniline (15.1 mg, 162 μmol) and HATU (77.0 mg, 202 μmol) were added and the mixture was stirred overnight at rt. The mixture was diluted with water and extracted 3× with ethyl acetate. The combined organic layers were dried and evaporated. The residue was purified by preparative HPLC to give 35.0 mg (100% purity, 58% yield) of the title compound.

LC-MS (Method 1): Rt=0.95 min; MS (ESIpos): m/z=446 [M+H]⁺

¹H-NMR (400 MHz, DMSO-d6) δ [ppm]: 1.002 (7.06), 1.020 (16.00), 1.038 (7.15), 1.243 (0.85), 1.258 (0.88), 1.263 (0.87), 1.280 (0.72), 2.050 (1.34), 2.054 (1.37), 2.066 (2.16), 2.074 (2.80), 2.083 (1.28), 2.090 (1.20), 2.518 (1.18), 2.524 (2.63), 2.542 (3.84), 2.559 (2.55), 3.021 (0.88), 3.039 (2.76), 3.053 (3.04), 3.057 (2.99), 3.070 (2.70), 3.088 (0.82), 3.382 (1.64), 3.391 (1.42), 3.400 (1.84), 3.418 (8.20), 3.445 (1.21), 3.473 (0.83), 3.490 (1.49), 3.498 (0.81), 3.506 (0.96), 3.515 (0.89), 4.177 (2.48), 4.195 (3.89), 4.212 (2.37), 6.159 (1.15), 6.173 (2.27), 6.186 (1.10), 6.446 (11.17), 7.070 (3.42), 7.092 (1.22), 7.094 (1.77), 7.098 (1.08), 7.113 (3.27), 7.129 (1.20), 7.131 (1.90), 7.134 (1.15), 7.336 (3.83), 7.341 (1.38), 7.357 (5.04), 7.371 (1.20), 7.376 (3.52), 7.690 (4.59), 7.692 (5.23), 7.707 (1.45), 7.711 (4.84), 7.714 (3.75), 8.354 (4.30), 8.360 (4.59), 8.545 (6.17), 8.551 (5.58), 10.318 (4.08).

### Example 553

### (rac)-2′-[6-amino-5-(cyclohexylcarbamoyl)pyridin-3-yl]-N-ethyl-5′,6′-dihydrospiro[pyrrolidine-3,4′-pyrrolo[1,2-b]pyrazole]-1-carboxamide

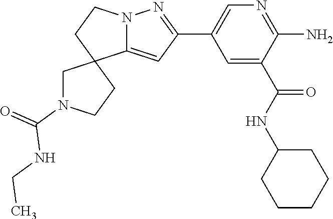 [SMILES: CCNC(=O)N1CCC2(CCn3nc(-c4cnc(N)c(C(=O)NC5CCCCC5)c4)cc32)C1]

2-amino-5-[(rac)-1-(ethylcarbamoyl)-5′,6′-dihydrospiro[pyrrolidine-3,4′-pyrrolo[1,2-b]pyrazol]-2′-yl]pyridine-3-carboxylic acid (75.0 mg, 202 μmol) was solubilised in THF (1.5 mL), cyclohexanamine (28 μL, 240 μmol), HATU (115 mg, 304 μmol) and triethylamine (71 μL, 510 μmol) were added and the mixture was stirred for 3 h at rt. cyclohexanamine (28 μL, 240 μmol) and N-methylpyrrolidone (250 μL, 2.6 mmol) were added and the mixture was stirred overnight at rt. cyclohexanamine (28 μL, 240 μmol), HATU (115 mg, 304 μmol) and triethylamine (71 μL, 510 μmol) were added and the mixture was stirred at 80° C. The mixture was diluted with water and extracted 3× with ethyl acetate. The combined organic layers were dried and evaporated. The residue was purified by preparative HPLC to give 2.00 mg (98% purity, 2% yield) of the title compound.

LC-MS (Method 1): Rt=0.98 min; MS (ESIpos): m/z=452 [M+H]⁺

¹H-NMR (400 MHz, DMSO-d6) δ [ppm]: 1.004 (7.08), 1.022 (16.00), 1.040 (7.13), 1.111 (0.55), 1.133 (0.65), 1.242 (0.40), 1.275 (1.25), 1.296 (2.69), 1.308 (3.19), 1.337 (1.50), 1.370 (0.40), 1.600 (0.90), 1.630 (0.80), 1.734 (1.50), 1.746 (1.84), 1.806 (2.04), 2.042 (1.20), 2.057 (1.89), 2.069 (2.44), 2.073 (2.59), 2.084 (3.34), 2.099 (0.45), 2.332 (0.60), 2.518 (4.49), 2.523 (2.99), 2.532 (4.24), 2.539 (2.64), 2.550 (2.74), 3.022 (0.85), 3.039 (2.84), 3.053 (3.09), 3.057 (3.04), 3.071 (2.79), 3.089 (0.80), 3.377 (0.85), 3.383 (1.10), 3.395 (1.50), 3.408 (6.48), 3.413 (6.38), 3.420 (2.54), 3.438 (1.35), 3.464 (0.75), 3.482 (1.40), 3.489 (0.80), 3.497 (0.95), 3.507 (0.80), 3.523 (0.45), 3.739 (0.65), 3.748 (0.70), 3.757 (0.70), 4.164 (2.49), 4.181 (3.99), 4.199 (2.44), 6.154 (1.15), 6.168 (2.29), 6.182 (1.15), 6.375 (11.51), 7.079 (4.88), 8.167 (3.99), 8.172 (4.24), 8.338 (1.89), 8.358 (1.84), 8.458 (5.43), 8.463 (5.33).

### Example 554

### (rac)-2′-[6-amino-5-(diethylcarbamoyl)pyridin-3-yl]-N-ethyl-5′,6′-dihydrospiro[pyrrolidine-3,4′-pyrrolo[1,2-b]pyrazole]-1-carboxamide

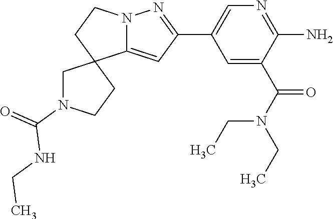 [SMILES: CCNC(=O)N1CCC2(CCn3nc(-c4cnc(N)c(C(=O)N(CC)CC)c4)cc32)C1]

2-amino-5-[(rac)-1-(ethylcarbamoyl)-5′,6′-dihydrospiro[pyrrolidine-3,4′-pyrrolo[1,2-b]pyrazol]-2′-yl]pyridine-3-carboxylic acid (100 mg, 270 μmol) was solubilised in THF (2.0 mL), N-ethylethanamine (34 μL, 320 μmol), HATU (154 mg, 405 μmol), N-methylpyrrolidone (330 μL, 3.5 mmol) and triethylamine (94 μL, 670 μmol) were added and the mixture was stirred for 3 h at 80° C. The mixture was diluted with water and extracted 3× with ethyl acetate. The combined organic layers were dried and evaporated. The residue was purified by preparative HPLC to give 2.80 mg (100% purity, 2% yield) of the title compound.

LC-MS (Method 1): Rt=0.80 min; MS (ESIpos): m/z=426 [M+H]⁺

¹H-NMR (600 MHz, DMSO-d6) δ [ppm]: 1.006 (7.73), 1.018 (16.00), 1.030 (7.69), 1.066 (1.15), 1.099 (3.29), 1.599 (0.81), 2.030 (1.43), 2.042 (1.75), 2.051 (1.33), 2.058 (2.29), 2.070 (1.20), 2.078 (0.83), 2.513 (4.64), 2.515 (4.73), 2.525 (3.16), 2.539 (0.74), 3.031 (1.09), 3.042 (3.46), 3.052 (3.57), 3.054 (3.73), 3.064 (3.01), 3.075 (0.97), 3.289 (0.87), 3.301 (1.06), 3.365 (2.59), 3.378 (2.71), 3.383 (2.42), 3.391 (2.00), 3.396 (3.71), 3.401 (11.35), 3.418 (0.84), 3.478 (0.95), 3.487 (1.16), 3.491 (1.41), 3.495 (1.12), 3.500 (1.22), 3.504 (1.06), 3.507 (1.00), 3.517 (0.73), 4.152 (3.22), 4.165 (5.58), 4.176 (3.22), 5.857 (1.20), 5.866 (7.02), 6.139 (1.24), 6.149 (2.38), 6.158 (1.25), 6.386 (10.60), 7.647 (4.93), 7.650 (5.20), 8.310 (6.02), 8.393 (5.42), 8.397 (5.44).

### Example 555

### (rac)-2′-(5-acetyl-6-aminopyridin-3-yl)-N-ethyl-5′,6′-dihydrospiro[pyrrolidine-3,4′-pyrrolo[1,2-b]pyrazole]-1-carboxamide

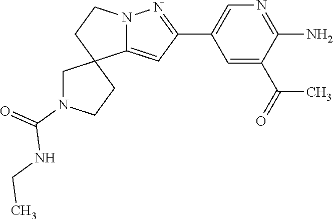 [SMILES: CCNC(=O)N1CCC2(CCn3nc(-c4cnc(N)c(C(C)=O)c4)cc32)C1]

(rac)-1-(ethylcarbamoyl)-5′,6′-dihydrospiro[pyrrolidine-3,4′-pyrrolo[1,2-b]pyrazol]-2′-yl trifluoromethanesulfonate (100 mg, 262 μmol), 5-acetyl-6-aminopyridin-3-yl)boronic acid (51 mg, 288 μmol), Na2CO3 (83.2 mg, 785 μmol) and Pd(dppf)2Cl2 (3.83 mg, 5.23 μmol) were stirred in a mixture of 1,2-dimethoxyethane (700 μl), ethanol (300 μl) and water (200 μl) under argon for 1 h at 120° C. The mixture was diluted with brine, extracted with ethyl acetate and the organic layer was dried over a silicone filter and concentrated under reduced pressure. The residue was purified by preparative HPLC to give 11.2 mg (97% purity, 12% yield) of the title compound.

LC-MS (Method 1): Rt=0.74 min; MS (ESIpos): m/z=369 [M+H]⁺

¹H-NMR (400 MHz, DMSO-d6) δ [ppm]: 1.004 (4.21), 1.022 (10.02), 1.040 (4.50), 2.048 (1.05), 2.066 (2.10), 2.083 (1.05), 2.523 (10.62), 2.541 (4.28), 2.560 (2.44), 2.605 (0.84), 2.619 (16.00), 3.041 (1.12), 3.054 (1.27), 3.071 (1.15), 3.388 (1.29), 3.414 (5.12), 3.417 (4.90), 3.430 (2.77), 3.447 (2.56), 4.182 (1.58), 4.200 (2.58), 4.217 (1.65), 6.167 (0.72), 6.525 (6.03), 6.618 (0.22), 7.856 (0.24), 8.470 (1.89), 8.476 (2.10), 8.616 (3.73), 8.622 (3.59).

### Example 556

### 5-{1-[1-(1H-imidazol-2-yl)ethyl]-5′,6′-dihydrospiro[pyrrolidine-3,4′-pyrrolo[1,2-b]pyrazol]-2′-yl}-3-(trifluoromethyl)pyridin-2-amine (Mixture of Stereoisomers)

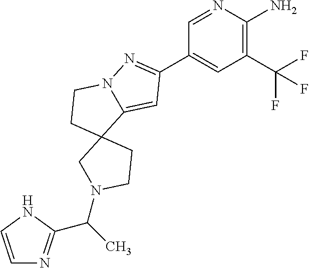 [SMILES: CC(c1ncc[nH]1)N1CCC2(CCn3nc(-c4cnc(N)c(C(F)(F)F)c4)cc32)C1]

5-[5′,6′-dihydrospiro[pyrrolidine-3,4′-pyrrolo[1,2-b]pyrazol]-2′-yl]-3-(trifluoromethyl)pyridin-2-amine-hydrochloride salt (100 mg, 84% purity, 233 μmol, stereoisomer 1), 1-(1H-imidazol-2-yl)ethan-1-one (38.6 mg, 350 μmol), titanium(IV) isopropoxide (210 μL, 700 μmol) and N,N-diisopropylethylamine (160 μL) were stirred in methanol (2.5 mL) for 2 h at 60° C. NaBH3CN (14.7 mg, 233 μmol) was added and the mixture was stirred at rt. The mixture was filtered and the filtrate was evaporated. The residue was purified by preparative HPLC to give 7.00 mg (90% purity, 6% yield) of the title compound.

1H NMR (DMSO-d6, 400 MHz) δ 10.5-13.1 (m, 1H), 8.5-8.7 (m, 1H), 8.0-8.0 (m, 1H), 7.0-7.1 (m, 2H), 6.5-6.6 (m, 2H), 6.3-6.4 (m, 1H), 4.0-4.1 (m, 2H), 3.8-3.9 (m, 1H), 2.8-2.9 (m, 1H), 2.7-2.8 (m, 1H), 2.6-2.7 (m, 2H), 2.5-2.6 (m, 1H), 1.9-2.0 (m, 2H), 1.4-1.4 (m, 3H)

LC-MS (method 1): Rₜ=0.87 min; MS (ESIpos): m/z=419 [M+H]⁺

### Example 557

### 5-{1-[1-(1H-pyrazol-3-yl)ethyl]-5′,6′-dihydrospiro[pyrrolidine-3,4′-pyrrolo[1,2-b]pyrazol]-2′-yl}-3-(trifluoromethyl)pyridin-2-amine (Mixture of Stereoisomers)

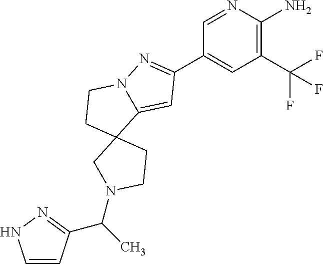 [SMILES: CC(c1cc[nH]n1)N1CCC2(CCn3nc(-c4cnc(N)c(C(F)(F)F)c4)cc32)C1]

Example 557 was prepared in analogy to Example 556 using 5-[5′,6′-dihydrospiro[pyrrolidine-3,4′-pyrrolo[1,2-b]pyrazol]-2′-yl]-3-(trifluoromethyl)pyridin-2-amine-hydrochloride salt (75.0 mg, 208 μmol, stereoisomer 1) and 1-(1H-pyrazol-3-yl)ethan-1-one-hydrochloride salt (45.8 mg, 313 μmol).

1H NMR (DMSO-d6, 400 MHz) δ 12.4-12.7 (m, 1H), 8.5-8.6 (m, 1H), 7.8-8.2 (m, 1H), 7.3-7.7 (m, 1H), 6.5-6.6 (m, 2H), 6.2-6.4 (m, 1H), 5.9-6.2 (m, 1H), 4.0-4.2 (m, 2H), 3.6-3.8 (m, 1H), 2.7-2.8 (m, 1H), 2.61 (br d, 3H), 2.4-2.5 (m, 1H), 1.8-2.1 (m, 2H), 1.35 (d, 3H)

[α]²⁰_{D}: −33.9° (c=1.00, DMSO)

LC-MS (method 1): Rₜ=0.96 min; MS (ESIpos): m/z=419 [M+H]⁺

### Example 558

### 5-{(3R)-1-[1-(1H-pyrazol-3-yl)ethyl]-5′,6′-dihydrospiro[pyrrolidine-3,4′-pyrrolo[1,2-b]pyrazol]-2′-yl}-3-(trifluoromethyl)pyridin-2-amine (Mixture of Stereoisomers)

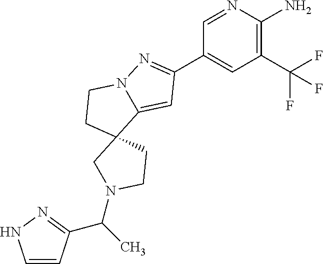 [SMILES: CC(c1cc[nH]n1)N1CC[C@@]2(CCn3nc(-c4cnc(N)c(C(F)(F)F)c4)cc32)C1]

Example 558 was prepared in analogy to Example 556 using 5-[(3R)-5′,6′-dihydrospiro[pyrrolidine-3,4′-pyrrolo[1,2-b]pyrazol]-2′-yl]-3-(trifluoromethyl)pyridin-2-amine-hydrochloride salt (100 mg, 278 μmol) and 1-(1H-pyrazol-3-yl)ethan-1-one-hydrochloride salt (61.1 mg, 417 μmol).

1H NMR (DMSO-d6) δ: 12.34-12.85 (m, 1H), 8.52-8.67 (m, 1H), 7.92-8.12 (m, 1H), 7.28-7.70 (m, 1H), 6.51 (s, 2H), 6.38 (d, 1H), 6.16 (s, 1H), 3.96-4.17 (m, 2H), 3.68 (br s, 1H), 2.69-2.77 (m, 1H), 2.57-2.66 (m, 2H), 2.41-2.49 (m, 2H), 1.87-2.02 (m, 2H), 1.35 (d, 3H)

[α]²⁰_{D}: +37.6° (c=1.00, DMSO)

LC-MS (method 1): Rₜ=0.94 min; MS (ESIpos): m/z=419 [M+H]⁺

### Example 559

### 5-{(3R)-1-[1-(1H-imidazol-2-yl)ethyl]-5′,6′-dihydrospiro[pyrrolidine-3,4′-pyrrolo[1,2-b]pyrazol]-2′-yl}-3-(trifluoromethyl)pyridin-2-amine (Mixture of Stereoisomers)

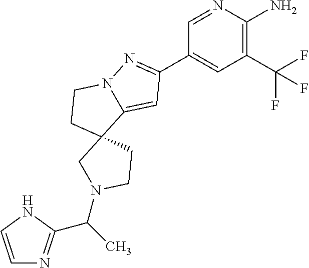 [SMILES: CC(c1ncc[nH]1)N1CC[C@@]2(CCn3nc(-c4cnc(N)c(C(F)(F)F)c4)cc32)C1]

Example 559 was prepared in analogy to Example 556 using 5-[(3R)-5′,6′-dihydrospiro[pyrrolidine-3,4′-pyrrolo[1,2-b]pyrazol]-2′-yl]-3-(trifluoromethyl)pyridin-2-amine-hydrochloride salt (100 mg, 278 μmol) and 1-(1H-imidazol-2-yl)ethan-1-one (45.9 mg, 417 μmol).

1H NMR (DMSO-d6) δ: 11.77 (br s, 1H), 8.58 (s, 1H), 8.00 (d, 1H), 7.02 (br s, 1H), 6.78 (br s, 1H), 6.52 (s, 2H), 6.34-6.43 (m, 1H), 4.05-4.13 (m, 2H), 3.67-3.76 (m, 1H), 2.66-2.89 (m, 2H), 2.54-2.64 (m, 2H), 2.40-2.49 (m, 2H), 1.88-2.02 (m, 2H), 1.38 (d, 3H)

[α]²⁰_{D}: +39.4° (c=1.00, DMSO)

LC-MS (method 1): Rₜ=0.87 min; MS (ESIpos): m/z=418 [M+H]⁺

### Example 560

### 5-{(3R)-1-[1-(1H-imidazol-5-yl)ethyl]-5′,6′-dihydrospiro[pyrrolidine-3,4′-pyrrolo[1,2-b]pyrazol]-2′-yl}-3-(trifluoromethyl)pyridin-2-amine (Mixture of Stereoisomers)

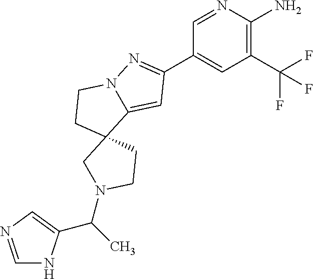 [SMILES: CC(c1cnc[nH]1)N1CC[C@@]2(CCn3nc(-c4cnc(N)c(C(F)(F)F)c4)cc32)C1]

Example 560 was prepared in analogy to Example 556 using 5-[(3R)-5′,6′-dihydrospiro[pyrrolidine-3,4′-pyrrolo[1,2-b]pyrazol]-2′-yl]-3-(trifluoromethyl)pyridin-2-amine-hydrochloride salt (100 mg, 278 μmol) and 1-(1H-imidazol-5-yl)ethan-1-one (45.9 mg, 417 μmol).

1H NMR (DMSO-d6) δ: 11.76-12.04 (m, 1H), 8.52-8.65 (m, 1H), 8.00 (d, 1H), 7.52 (s, 1H), 6.67-6.99 (m, 1H), 6.51 (br s, 2H), 6.31-6.44 (m, 1H), 4.07 (m, 2H), 3.55-3.69 (m, 1H), 2.55-2.92 (m, 4H), 1.88-2.03 (m, 2H), 1.34 (br m, 3H)

[α]²⁰_{D}: +47.3° (c=1.00, DMSO)

LC-MS (method 1): Rₜ=0.86 min; MS (ESIpos): m/z=419 [M+H]⁺

### Example 561

### 5-{(3R)-1-[1-(1-methyl-1H-pyrazol-3-yl)ethyl]-5′,6′-dihydrospiro[pyrrolidine-3,4′-pyrrolo[1,2-b]pyrazol]-2′-yl}-3-(trifluoromethyl)pyridin-2-amine (Mixture of Stereoisomers)

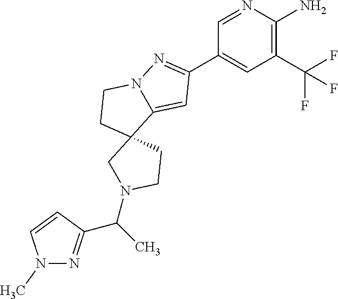 [SMILES: CC(c1ccn(C)n1)N1CC[C@@]2(CCn3nc(-c4cnc(N)c(C(F)(F)F)c4)cc32)C1]

Example 561 was prepared in analogy to Example 556 using 5-[(3R)-5′,6′-dihydrospiro[pyrrolidine-3,4′-pyrrolo[1,2-b]pyrazol]-2′-yl]-3-(trifluoromethyl)pyridin-2-amine-hydrochloride salt (100 mg, 278 μmol) and 1-(1-methyl-1H-pyrazol-3-yl)ethan-1-one (51.8 mg, 417 μmol).

1H NMR (DMSO-d6) δ: 8.58 (m, 1H), 8.01 (m, 1H), 7.56 (m, 1H), 6.51 (s, 2H), 6.39 (d, 1H), 6.14 (d, 1H), 4.02-4.13 (m, 2H), 3.76 (d, 3H), 3.58 (m, 1H), 2.68-2.76 (m, 1H), 2.53-2.68 (m, 3H), 2.40-2.48 (m, 1H), 1.89-2.03 (m, 2H), 1.25-1.40 (m, 3H)

[α]²⁰_{D}: +43.0° (c=1.00, DMSO)

LC-MS (method 1): Rₜ=1.00 min; MS (ESIpos): m/z=433 [M+H]⁺

### Example 562

### 5-{(3R)-1-[1-(1H-pyrazol-4-yl)ethyl]-5′,6′-dihydrospiro[pyrrolidine-3,4′-pyrrolo[1,2-b]pyrazol]-2′-yl}-3-(trifluoromethyl)pyridin-2-amine (Mixture of Stereoisomers)

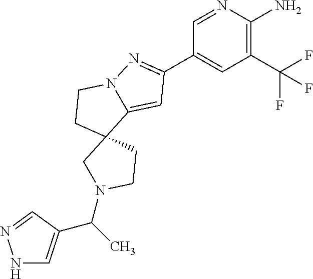 [SMILES: CC(c1cn[nH]c1)N1CC[C@@]2(CCn3nc(-c4cnc(N)c(C(F)(F)F)c4)cc32)C1]

Example 562 was prepared in analogy to Example 556 using 5-[(3R)-5′,6′-dihydrospiro[pyrrolidine-3,4′-pyrrolo[1,2-b]pyrazol]-2′-yl]-3-(trifluoromethyl)pyridin-2-amine-hydrochloride salt (100 mg, 278 μmol) and 1-(1H-pyrazol-4-yl)ethan-1-one (45.9 mg, 417 μmol).

1H NMR (DMSO-d6) δ: 12.61 (s, 1H), 8.47-8.68 (m, 1H), 8.01 (m, 1H), 7.51 (br s, 2H), 6.51 (s, 2H), 6.37 (d, 1H), 4.03-4.13 (m, 2H), 3.58 (qd, 1H), 2.61-2.72 (m, 2H), 2.56 (d, 1H), 2.39-2.48 (m, 1H), 1.87-2.03 (m, 2H), 1.33 (d, 3H)

[α]²⁰_{D}: +55.0° (c=1.00, DMSO)

LC-MS (method 1): Rₜ=0.92 min; MS (ESIpos): m/z=419 [M+H]⁺

### Example 563

### 5-{(3R)-1-[1-(4-methyl-1H-imidazol-2-yl)ethyl]-5′,6′-dihydrospiro[pyrrolidine-3,4′-pyrrolo[1,2-b]pyrazol]-2′-yl}-3-(trifluoromethyl)pyridin-2-amine (mixture of stereoisomers)

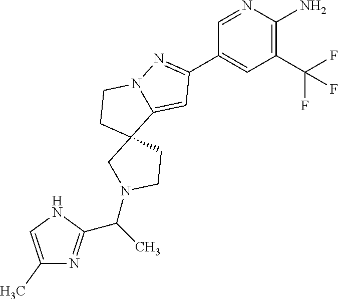 [SMILES: Cc1c[nH]c(C(C)N2CC[C@@]3(CCn4nc(-c5cnc(N)c(C(F)(F)F)c5)cc43)C2)n1]

Example 563 was prepared in analogy to Example 556 using 5-[(3R)-5′,6′-dihydrospiro[pyrrolidine-3,4′-pyrrolo[1,2-b]pyrazol]-2′-yl]-3-(trifluoromethyl)pyridin-2-amine-hydrochloride salt (125 mg, 347 μmol) and 1-(4-methyl-1H-imidazol-2-yl)ethanone (65 mg, 521 μmol).

1H NMR (DMSO-d6) δ: 11.32-11.57 (m, 1H), 8.58 (d, 1H), 7.89-8.05 (m, 1H), 6.59-6.77 (m, 0.6H), 6.52 (s, 2H), 6.40-6.47 (m, 0.4H), 6.32-6.39 (m, 1H), 4.03-4.15 (m, 2H), 3.56-3.68 (m, 1H), 2.81 (br d, 1H), 2.65-2.78 (m, 1H), 2.52-2.65 (m, 3H), 2.41-2.48 (m, 1H), 2.02-2.16 (br m, 3H), 1.88-2.01 (m, 2H), 1.35 (d, 3H)

[α]²⁰_{D}: +41.9° (c=1.00, DMSO)

LC-MS (method 1): Rₜ=0.91 min; MS (ESIpos): m/z=433 [M+H]⁺

### Example 564

### 5-{(3R)-1-[1-(3-methyl-1H-pyrazol-5-yl)ethyl]-5′,6′-dihydrospiro[pyrrolidine-3,4′-pyrrolo[1,2-b]pyrazol]-2′-yl}-3-(trifluoromethyl)pyridin-2-amine (mixture of stereoisomers)

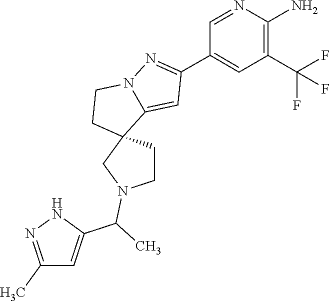 [SMILES: Cc1cc(C(C)N2CC[C@@]3(CCn4nc(-c5cnc(N)c(C(F)(F)F)c5)cc43)C2)[nH]n1]

Example 564 was prepared in analogy to Example 556 using 5-[(3R)-5′,6′-dihydrospiro[pyrrolidine-3,4′-pyrrolo[1,2-b]pyrazol]-2′-yl]-3-(trifluoromethyl)pyridin-2-amine-hydrochloride salt (125 mg, 347 μmol) and 1-(3-methyl-1H-pyrazol-5-yl)ethan-1-one (64.7 mg, 521 μmol).

1H NMR (DMSO-d6, 400 MHz) δ 12.0-12.3 (m, 1H), 8.58 (t, 1H), 7.9-8.1 (m, 1H), 6.52 (s, 2H), 6.3-6.4 (m, 1H), 5.90 (s, 1H), 4.0-4.1 (m, 2H), 3.5-3.7 (m, 1H), 2.7-2.8 (m, 1H), 2.5-2.7 (m, 3H), 2.16 (br s, 3H), 1.9-2.0 (m, 2H), 1.31 (br d, 3H)

[α]²⁰_{D}: +42.2° (c=1.00, DMSO)

LC-MS (method 1): Rₜ=0.99 min; MS (ESIpos): m/z=433 [M+H]⁺

### Example 565

### 5-{(3R)-1-[1-(4-methyl-1H-pyrazol-5-yl)ethyl]-5′,6′-dihydrospiro[pyrrolidine-3,4′-pyrrolo[1,2-b]pyrazol]-2′-yl}-3-(trifluoromethyl)pyridin-2-amine (mixture of stereoisomers)

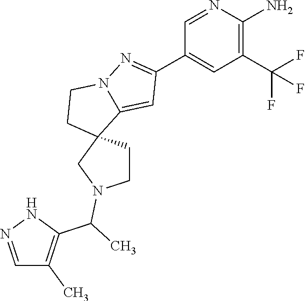 [SMILES: Cc1cn[nH]c1C(C)N1CC[C@@]2(CCn3nc(-c4cnc(N)c(C(F)(F)F)c4)cc32)C1]

Example 565 was prepared in analogy to Example 556 using 5-[(3R)-5′,6′-dihydrospiro[pyrrolidine-3,4′-pyrrolo[1,2-b]pyrazol]-2′-yl]-3-(trifluoromethyl)pyridin-2-amine-hydrochloride salt (125 mg, 347 μmol) and 1-(4-methyl-1H-pyrazol-5-yl)ethan-1-one (64.7 mg, 521 μmol).

1H NMR (DMSO-d6, 400 MHz) δ 12.1-12.5 (m, 1H), 8.57 (s, 1H), 8.00 (d, 1H), 7.1-7.5 (m, 1H), 6.52 (s, 2H), 6.41 (br s, 1H), 4.0-4.2 (m, 2H), 3.61 (quin, 1H), 2.73 (br d, 1H), 2.5-2.6 (m, 2H), 1.9-2.1 (m, 5H), 1.34 (br d, 3H)

[α]²⁰_{D}: +45.5° (c=1.00, DMSO)

LC-MS (method 1): Rₜ=1.05 min; MS (ESIpos): m/z=433 [M+H]⁺

### Example 566

### 5-[(3S)-1-(pyridin-3-ylmethyl)-5′,6′-dihydrospiro[pyrrolidine-3,4′-pyrrolo[1,2-b]pyrazol]-2′-yl]-3-(trifluoromethyl)pyridin-2-amine

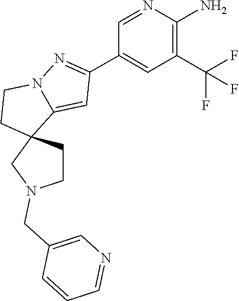 [SMILES: Nc1ncc(-c2cc3n(n2)CC[C@]32CCN(Cc3cccnc3)C2)cc1C(F)(F)F]

5-[(3S)-5′,6′-dihydrospiro[pyrrolidine-3,4′-pyrrolo[1,2-b]pyrazol]-2′-yl]-3-(trifluoromethyl)pyridin-2-amine-hydrochloride salt (100 mg, 84% purity, 233 μmol) and pyridine-3-carbaldehyde (33 μL, 350 μmol) were solubilised in THF (2.5 mL), acetic acid (20 μL) was added and the mixture was stirred for 30 min at rt. Na(CH3000)3BH was added and the mixture was stirred overnight at rt. It was diluted with water and extracted 2× with ethyl acetate. The combined organic layers were washed with brine, dried over Na₂SO₄ and concentrated under reduced pressure. The residue was purified by preparative HPLC to give 14.0 mg (99% purity, 14% yield) of the title compound.

1H NMR (DMSO-d6, 400 MHz) δ 8.6-8.6 (m, 1H), 8.5-8.6 (m, 1H), 8.4-8.5 (m, 1H), 7.9-8.1 (m, 1H), 7.6-7.8 (m, 1H), 7.3-7.4 (m, 1H), 6.5-6.6 (m, 2H), 6.48 (s, 1H), 4.0-4.2 (m, 2H), 3.6-3.8 (m, 2H), 2.5-2.8 (m, 6H), 2.0-2.1 (m, 2H)

[α]²⁰_{D}: −53.2° (c=1.00, DMSO)

LC-MS (method 1): Rₜ=1.01 min; MS (ESIpos): m/z=415 [M+H]⁺

### Example 567

### 5-[(3R)-1-(pyridin-3-ylmethyl)-5′,6′-dihydrospiro[pyrrolidine-3,4′-pyrrolo[1,2-b]pyrazol]-2′-yl]-3-(trifluoromethyl)pyridin-2-amine

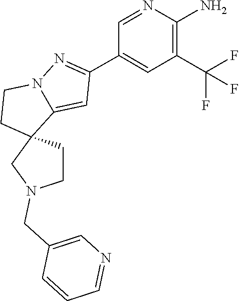 [SMILES: Nc1ncc(-c2cc3n(n2)CC[C@@]32CCN(Cc3cccnc3)C2)cc1C(F)(F)F]

Example 567 was prepared in analogy to Example 566 using 5-[(3R9-5′,6′-dihydrospiro[pyrrolidine-3,4′-pyrrolo[1,2-b]pyrazol]-2′-yl]-3-(trifluoromethyl)pyridin-2-amine-hydrochloride salt (100 mg, 82% purity, 228 μmol) and pyridine-3-carbaldehyde (32 μL, 340 μmol).

1H NMR (DMSO-d6, 400 MHz) δ 8.6-8.6 (m, 1H), 8.5-8.6 (m, 1H), 8.4-8.5 (m, 1H), 8.0-8.0 (m, 1H), 7.7-7.8 (m, 1H), 7.3-7.4 (m, 1H), 6.5-6.6 (m, 2H), 6.5-6.5 (m, 1H), 4.0-4.1 (m, 2H), 3.6-3.7 (m, 2H), 2.5-2.8 (m, 6H), 2.0-2.1 (m, 2H)

[α]²⁰_{D}: +50.4° (c=1.00, DMSO)

LC-MS (method 1): Rₜ=1.01 min; MS (ESIpos): m/z=415 [M+H]⁺

### Example 568

### 5-{(rac)-1-[(1H-pyrazol-3-yl)methyl]-5′,6′-dihydrospiro[pyrrolidine-3,4′-pyrrolo[1,2-b]pyrazol]-2′-yl}-3-(trifluoromethyl)pyridin-2-amine

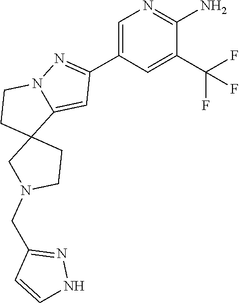 [SMILES: Nc1ncc(-c2cc3n(n2)CCC32CCN(Cc3cc[nH]n3)C2)cc1C(F)(F)F]

Example 568 was prepared in analogy to Example 566 using 5-[(rac)-5′,6′-dihydrospiro[pyrrolidine-3,4′-pyrrolo[1,2-b]pyrazol]-2′-yl]-3-(trifluoromethyl)pyridin-2-amine-hydrochloride salt (200 mg, 556 μmol) and 1H-pyrazole-3-carbaldehyde (80.1 mg, 834 μmol).

1H NMR (DMSO-d6, 400 MHz) δ 12.5-12.7 (m, 1H), 8.6-8.6 (m, 1H), 8.01 (d, 1H), 7.3-7.7 (m, 1H), 6.51 (s, 2H), 6.4-6.5 (m, 1H), 6.18 (br s, 1H), 4.1-4.1 (m, 2H), 3.6-3.7 (m, 2H), 2.7-2.8 (m, 1H), 2.6-2.7 (m, 4H), 2.0-2.1 (m, 2H)

LC-MS (method 1): Rₜ=0.90 min; MS (ESIpos): m/z=405 [M+H]⁺

### Example 569

### 5-[(3S)-1-(1H-imidazol-2-ylmethyl)-5′,6′-dihydrospiro[pyrrolidine-3,4′-pyrrolo[1,2-b]pyrazol]-2′-yl]-3-(trifluoromethyl)pyridin-2-amine

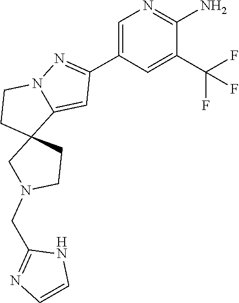 [SMILES: Nc1ncc(-c2cc3n(n2)CC[C@]32CCN(Cc3ncc[nH]3)C2)cc1C(F)(F)F]

Example 569 was prepared in analogy to Example 566 using 5-[(3S9-5′,6′-dihydrospiro[pyrrolidine-3,4′-pyrrolo[1,2-b]pyrazol]-2′-yl]-3-(trifluoromethyl)pyridin-2-amine-hydrochloride salt (100 mg, 84% purity, 233 μmol) and 1H-imidazole-2-carbaldehyde (33.7 mg, 350 μmol).

1H NMR (DMSO-d6, 400 MHz) δ 11.5-12.1 (m, 1H), 8.5-8.7 (m, 1H), 7.9-8.1 (m, 1H), 6.8-7.1 (m, 2H), 6.52 (s, 2H), 6.46 (s, 1H), 4.1-4.1 (m, 2H), 3.67 (s, 2H), 2.7-2.8 (m, 2H), 2.6-2.7 (m, 2H), 2.5-2.6 (m, 1H), 2.0-2.1 (m, 2H)

[α]²⁰_{D}: −45.5° (c=1.00, DMSO)

LC-MS (method 1): Rₜ=0.83 min; MS (ESIpos): m/z=404 [M+H]⁺

### Example 570

### 5-[(3R)-1-(1H-imidazol-2-ylmethyl)-5′,6′-dihydrospiro[pyrrolidine-3,4′-pyrrolo[1,2-b]pyrazol]-2′-yl]-3-(trifluoromethyl)pyridin-2-amine

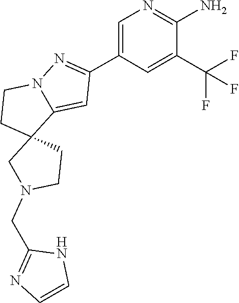 [SMILES: Nc1ncc(-c2cc3n(n2)CC[C@@]32CCN(Cc3ncc[nH]3)C2)cc1C(F)(F)F]

Example 570 was prepared in analogy to Example 566 using 5-[(3R)-5′,6′-dihydrospiro[pyrrolidine-3,4′-pyrrolo[1,2-b]pyrazol]-2′-yl]-3-(trifluoromethyl)pyridin-2-amine-hydrochloride salt (100 mg, 82% purity, 228 μmol) and 1H-imidazole-2-carbaldehyde (32.9 mg, 342 μmol).

1H NMR (DMSO-d6, 400 MHz) δ 9.6-11.8 (m, 1H), 8.5-8.7 (m, 1H), 7.9-8.1 (m, 1H), 6.8-7.0 (m, 2H), 6.5-6.6 (m, 2H), 6.4-6.5 (m, 1H), 4.0-4.1 (m, 2H), 3.6-3.7 (m, 2H), 2.7-2.8 (m, 2H), 2.6-2.7 (m, 2H), 2.5-2.6 (m, 1H), 1.9-2.1 (m, 2H)

[α]²⁰_{D}: +45.3° (c=1.00, DMSO)

LC-MS (method 1): Rₜ=0.83 min; MS (ESIpos): m/z=404 [M+H]⁺

### Example 571

### 5-{(rac)-1-[(4-methyl-1H-pyrazol-3-yl)methyl]-5′,6′-dihydrospiro[pyrrolidine-3,4′-pyrrolo[1,2-b]pyrazol]-2′-yl}-3-(trifluoromethyl)pyridin-2-amine

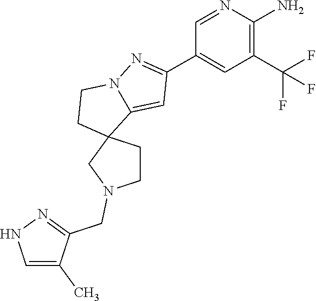 [SMILES: Cc1c[nH]nc1CN1CCC2(CCn3nc(-c4cnc(N)c(C(F)(F)F)c4)cc32)C1]

Example 571 was prepared in analogy to Example 566 using 5-[(rac)-5′,6′-dihydrospiro[pyrrolidine-3,4′-pyrrolo[1,2-b]pyrazol]-2′-yl]-3-(trifluoromethyl)pyridin-2-amine-hydrochloride salt (100 mg, 278 μmol) and 4-methyl-1H-pyrazole-3-carbaldehyde (45.9 mg, 417 μmol).

1H NMR (DMSO-d6) δ: 12.17-12.58 (m, 1H), 8.58 (d, 1H), 8.00 (d, 1H), 7.16-7.51 (m, 1H), 6.52 (s, 2H), 6.42 (s, 1H), 4.01-4.17 (m, 2H), 3.60 (s, 2H), 2.55-2.79 (m, 5H), 1.92-2.13 (m, 5H)

LC-MS (method 1): Rₜ=1.00 min; MS (ESIpos): m/z=419 [M+H]⁺

### Example 572

### 5-{(rac)-1-[(1H-pyrazol-4-yl)methyl]-5′,6′-dihydrospiro[pyrrolidine-3,4′-pyrrolo[1,2-b]pyrazol]-2′-yl}-3-(trifluoromethyl)pyridin-2-amine

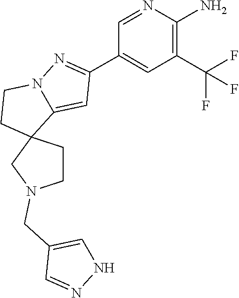 [SMILES: Nc1ncc(-c2cc3n(n2)CCC32CCN(Cc3cn[nH]c3)C2)cc1C(F)(F)F]

Example 572 was prepared in analogy to Example 566 using 5-[(rac)-5′,6′-dihydrospiro[pyrrolidine-3,4′-pyrrolo[1,2-b]pyrazol]-2′-yl]-3-(trifluoromethyl)pyridin-2-amine-hydrochloride salt (125 mg, 347 μmol) and 1H-pyrazole-4-carbaldehyde (50.1 mg, 521 μmol).

1H NMR (DMSO-d6) δ: 12.64 (br s, 1H), 8.59 (d, 1H), 8.02 (d, 1H), 7.33-7.74 (m, 2H), 6.51 (s, 2H), 6.44 (s, 1H), 4.09 (m, 2H), 3.53 (s, 2H), 2.67-2.78 (m, 2H), 2.53-2.66 (m, 3H), 1.96-2.07 (m, 2H)

LC-MS (method 1): Rₜ=0.88 min; MS (ESIpos): m/z=404 [M+H]⁺

### Example 573

### 5-{(rac)-1-[(1-methyl-1H-pyrazol-4-yl)methyl]-5′,6′-dihydrospiro[pyrrolidine-3,4′-pyrrolo[1,2-b]pyrazol]-2′-yl}-3-(trifluoromethyl)pyridin-2-amine

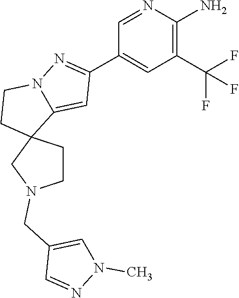 [SMILES: Cn1cc(CN2CCC3(CCn4nc(-c5cnc(N)c(C(F)(F)F)c5)cc43)C2)cn1]

Example 573 was prepared in analogy to Example 566 using 5-[(rac)-5′,6′-dihydrospiro[pyrrolidine-3,4′-pyrrolo[1,2-b]pyrazol]-2′-yl]-3-(trifluoromethyl)pyridin-2-amine-hydrochloride salt (125 mg, 347 μmol) and 1-methyl-1H-pyrazole-4-carbaldehyde (57.4 mg, 521 μmol).

1H NMR (DMSO-d6) δ: 8.59 (d, 1H), 8.02 (d, 1H), 7.58 (s, 1H), 7.33 (s, 1H), 6.51 (s, 2H), 6.43 (s, 1H), 4.08 (m, 2H), 3.78 (s, 3H), 3.48 (s, 2H), 2.67-2.77 (m, 2H), 2.53-2.64 (m, 3H), 1.94-2.07 (m, 2H)

LC-MS (method 1): Rₜ=0.94 min; MS (ESIpos): m/z=418 [M+H]⁺

### Example 574

### 5-{(rac)-1-[(1H-imidazol-4-yl)methyl]-5′,6′-dihydrospiro[pyrrolidine-3,4′-pyrrolo[1,2-b]pyrazol]-2′-yl}-3-(trifluoromethyl)pyridin-2-amine

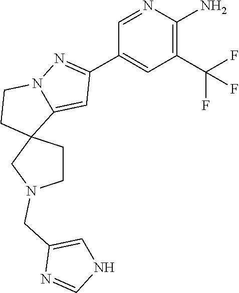 [SMILES: Nc1ncc(-c2cc3n(n2)CCC32CCN(Cc3c[nH]cn3)C2)cc1C(F)(F)F]

Example 574 was prepared in analogy to Example 566 using 5-[(rac)-5′,6′-dihydrospiro[pyrrolidine-3,4′-pyrrolo[1,2-b]pyrazol]-2′-yl]-3-(trifluoromethyl)pyridin-2-amine-hydrochloride salt (125 mg, 347 μmol) and 1H-imidazole-4-carbaldehyde (50.1 mg, 521 μmol).

1H NMR (DMSO-d6) δ: 8.58 (d, 1H), 8.01 (d, 1H), 7.62 (d, 1H), 7.00 (s, 1H), 6.53 (s, 2H), 6.46 (s, 1H), 4.10 (m, 2H), 3.73 (s, 2H), 2.78-2.98 (m, 4H), 2.55-2.64 (m, 1H), 1.99-2.09 (m, 2H)

LC-MS (method 1): Rₜ=0.83 min; MS (ESIneg): m/z=402 [M−H]⁻

### Example 575

### 5-{(rac)-1-[(1-methyl-1H-imidazol-4-yl)methyl]-5′,6′-dihydrospiro[pyrrolidine-3,4′-pyrrolo[1,2-b]pyrazol]-2′-yl}-3-(trifluoromethyl)pyridin-2-amine

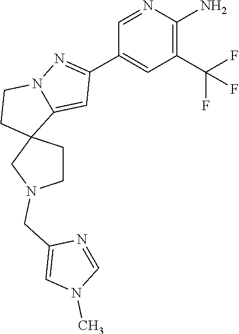 [SMILES: Cn1cnc(CN2CCC3(CCn4nc(-c5cnc(N)c(C(F)(F)F)c5)cc43)C2)c1]

Example 575 was prepared in analogy to Example 566 using 5-[(rac)-5′,6′-dihydrospiro[pyrrolidine-3,4′-pyrrolo[1,2-b]pyrazol]-2′-yl]-3-(trifluoromethyl)pyridin-2-amine-hydrochloride salt (125 mg, 347 μmol) and 1-methyl-1H-imidazole-4-carbaldehyde (57.4 mg, 521 μmol).

1H NMR (DMSO-d6) δ: 8.59 (d, 1H), 8.01 (d, 1H), 7.45 (d, 1H), 6.96 (d, 1H), 6.51 (s, 2H), 6.43 (s, 1H), 4.05-4.14 (m, 2H), 3.60 (s, 3H), 3.50 (s, 2H), 2.74-2.82 (m, 2H), 2.60-2.71 (m, 2H), 2.52-2.60 (m, 1H), 1.93-2.06 (m, 2H)

LC-MS (method 1): Rₜ=0.87 min; MS (ESIpos): m/z=419 [M+H]⁺

### Example 576

### 5-{(rac)-1-[(4-chloro-1H-pyrazol-5-yl)methyl]-5′,6′-dihydrospiro[pyrrolidine-3,4′-pyrrolo[1,2-b]pyrazol]-2′-yl}-3-(trifluoromethyl)pyridin-2-amine

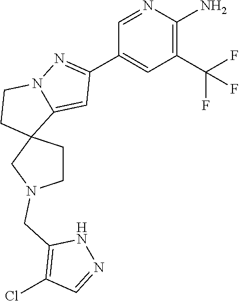 [SMILES: Nc1ncc(-c2cc3n(n2)CCC32CCN(Cc3[nH]ncc3Cl)C2)cc1C(F)(F)F]

Example 576 was prepared in analogy to Example 566 using 5-[(rac)-5′,6′-dihydrospiro[pyrrolidine-3,4′-pyrrolo[1,2-b]pyrazol]-2′-yl]-3-(trifluoromethyl)pyridin-2-amine-hydrochloride salt (125 mg, 347 μmol) and 4-chloro-1H-pyrazole-5-carbaldehyde (68.0 mg, 521 μmol).

1H NMR (DMSO-d6, 400 MHz) δ 12.9-13.2 (m, 1H), 8.58 (d, 1H), 8.00 (d, 1H), 7.5-8.0 (m, 1H), 6.52 (s, 2H), 6.4-6.5 (m, 1H), 4.09 (t, 2H), 3.6-3.8 (m, 2H), 2.7-2.8 (m, 2H), 2.6-2.7 (m, 3H), 2.0-2.1 (m, 2H)

LC-MS (method 1): Rₜ=1.04 min; MS (ESIpos): m/z=438 [M+H]⁺

### Example 577

### 3-[(2-fluorophenyl)methoxy]-5-{(rac)-1-[(1H-imidazol-2-yl)methyl]-5′,6′-dihydrospiro[pyrrolidine-3,4′-pyrrolo[1,2-b]pyrazol]-2′-yl}pyridin-2-amine

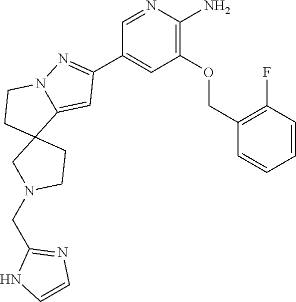 [SMILES: Nc1ncc(-c2cc3n(n2)CCC32CCN(Cc3ncc[nH]3)C2)cc1OCc1ccccc1F]

Example 577 was prepared in analogy to Example 566 using 5-[(rac)-5′,6′-dihydrospiro[pyrrolidine-3,4′-pyrrolo[1,2-b]pyrazol]-2′-yl]-3-[(2-fluorophenyl)methoxy]pyridin-2-amine-hydrochloride salt (104 mg, 230 μmol) and 1H-imidazole-2-carbaldehyde (24.3 mg, 253 μmol).

¹H-NMR (400 MHz, DMSO-d6) δ [ppm]: 1.157 (1.35), 1.232 (2.04), 1.974 (0.97), 1.988 (1.95), 2.007 (3.61), 2.024 (3.71), 2.043 (2.17), 2.326 (1.98), 2.563 (3.49), 2.580 (2.77), 2.596 (1.89), 2.613 (1.23), 2.642 (3.55), 2.665 (6.76), 2.698 (2.45), 2.717 (1.35), 2.741 (5.00), 2.765 (4.75), 2.787 (2.14), 2.809 (0.88), 3.674 (16.00), 4.067 (2.73), 4.080 (4.78), 4.095 (3.02), 5.208 (13.86), 5.749 (9.93), 6.355 (11.22), 6.823 (0.66), 7.231 (2.45), 7.241 (2.95), 7.249 (5.19), 7.266 (5.38), 7.286 (2.83), 7.397 (1.41), 7.411 (2.51), 7.431 (2.26), 7.446 (1.10), 7.479 (6.44), 7.653 (1.92), 7.671 (3.33), 7.690 (1.79), 7.961 (6.82), 7.965 (6.95), 11.854 (1.85).

LC-MS (Method 1): Rt=0.93 min; MS (ESIpos): m/z=460 [M+H]⁺

### Example 578

### 3-[(3-fluorophenyl)methoxy]-5-{(rac)-1-[(1H-imidazol-2-yl)methyl]-5′,6′-dihydrospiro[pyrrolidine-3,4′-pyrrolo[1,2-b]pyrazol]-2′-yl}pyridin-2-amine

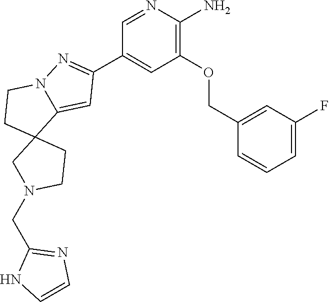 [SMILES: Nc1ncc(-c2cc3n(n2)CCC32CCN(Cc3ncc[nH]3)C2)cc1OCc1cccc(F)c1]

Example 578 was prepared in analogy to Example 566 using 5-[(rac)-5′,6′-dihydrospiro[pyrrolidine-3,4′-pyrrolo[1,2-b]pyrazol]-2′-yl]-3-[(3-fluorophenyl)methoxy]pyridin-2-amine-hydrochloride salt (86.0 mg, 190 μmol) and 1H-imidazole-2-carbaldehyde (20.1 mg, 209 μmol).

¹H-NMR (400 MHz, DMSO-d6) δ [ppm]: 1.166 (0.54), 1.201 (0.67), 1.232 (1.31), 1.954 (0.43), 1.973 (0.79), 1.985 (1.68), 2.004 (3.59), 2.020 (3.35), 2.039 (1.95), 2.051 (0.75), 2.071 (0.54), 2.327 (1.31), 2.331 (0.94), 2.449 (0.71), 2.466 (1.80), 2.544 (2.34), 2.560 (2.86), 2.578 (2.47), 2.592 (1.61), 2.610 (1.05), 2.639 (3.41), 2.661 (5.54), 2.669 (2.47), 2.674 (2.45), 2.678 (2.56), 2.697 (2.13), 2.717 (1.18), 2.737 (4.75), 2.751 (1.70), 2.761 (3.78), 2.786 (1.83), 2.807 (0.82), 3.505 (0.43), 3.674 (16.00), 4.054 (2.34), 4.060 (2.41), 4.072 (4.30), 4.089 (2.66), 4.094 (2.60), 5.204 (14.80), 5.855 (9.79), 6.334 (14.95), 6.920 (1.46), 7.126 (1.22), 7.131 (1.31), 7.153 (2.53), 7.169 (1.50), 7.174 (1.52), 7.351 (3.07), 7.370 (4.81), 7.412 (4.17), 7.427 (10.89), 7.431 (11.21), 7.447 (3.48), 7.467 (1.46), 7.946 (8.20), 7.950 (8.10), 11.855 (0.52).

LC-MS (Method 1): Rt=0.92 min; MS (ESIpos): m/z=460 [M+H]⁺

### Example 579

### 5-{(rac)-1-[(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine

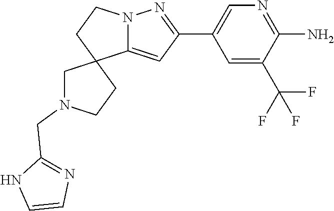 [SMILES: Nc1ncc(-c2cc3n(n2)CCC32CCN(Cc3ncc[nH]3)C2)cc1C(F)(F)F]

Example 579 was prepared in analogy to Example 566 using 5-[(rac)-5′,6′-dihydrospiro[pyrrolidine-3,4′-pyrrolo[1,2-b]pyrazol]-2′-yl]-3-(trifluoromethyl)pyridin-2-amine hydrochloride (75.0 mg, 208 μmol) and 1H-imidazole-2-carbaldehyde (15.0 mg, 156 μmol).

LC-MS (Method 1): Rt=0.84 min; MS (ESIneg): m/z=402 [M−H]⁻

¹H NMR (500 MHz, DMSO-d₆) δ ppm 2.00-2.11 (m, 2H) 2.52-2.54 (m, 3H) 2.57-2.63 (m, 1H) 2.72-2.89 (m, 3H) 3.80 (br s, 2H) 4.07-4.15 (m, 2H) 6.46 (s, 1H) 6.51 (s, 2H) 7.06 (br s, 2H) 8.00 (d, 1H) 8.58 (d, 1H)

### Example 580

### 5-{1-[(1H-imidazol-2-yl)methyl]-5′,6′-dihydrospiro[azetidine-3,4′-pyrrolo[1,2-b]pyrazol]-2′-yl}-3-(trifluoromethyl)pyridin-2-amine

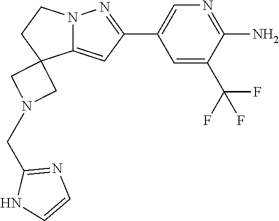

Example 580 was prepared in analogy to Example 566 using 5-(5′,6′-dihydrospiro[azetidine-3,4′-pyrrolo[1,2-b]pyrazol]-2′-yl)-3-(trifluoromethyl)pyridin-2-amine (75.0 mg, 242 μmol) and 1H-imidazole-2-carbaldehyde (17.5 mg, 182 μmol).

LC-MS (Method 1): Rt=0.81 min; MS (ESIpos): m/z=390 [M+H]⁺

¹H-NMR (400 MHz, DMSO-d6) δ [ppm]: 0.811 (0.28), 0.827 (0.28), 1.107 (3.18), 1.234 (0.23), 1.352 (0.23), 1.906 (0.51), 2.084 (1.31), 2.518 (16.00), 2.523 (11.13), 2.768 (0.51), 2.786 (0.89), 2.803 (0.61), 3.388 (2.34), 3.428 (1.45), 3.447 (0.84), 3.618 (2.29), 4.066 (0.56), 4.085 (0.89), 4.102 (0.56), 6.537 (1.36), 6.638 (2.25), 8.025 (0.70), 8.224 (0.37), 8.599 (0.75).

### Example 581

### 5-{(rac)-1′-[(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine

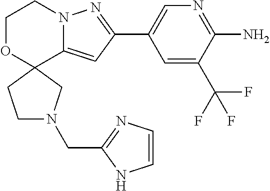 [SMILES: Nc1ncc(-c2cc3n(n2)CCOC32CCN(Cc3ncc[nH]3)C2)cc1C(F)(F)F]

Example 581 was prepared in analogy to Example 566 using 5-[(rac)-6,7-dihydrospiro[pyrazolo[5,1-c][1,4]oxazine-4,3′-pyrrolidin]-2-yl]-3-(trifluoromethyl)pyridin-2-amine (75.0 mg, 221 μmol) and 1H-imidazole-2-carbaldehyde (15.9 mg, 166 μmol).

LC-MS (Method 1): Rt=0.85 min; MS (ESIpos): m/z=420 [M+H]⁺

¹H-NMR (500 MHz, DMSO-d6) δ [ppm]: 1.001 (1.28), 1.015 (3.00), 1.029 (1.30), 1.909 (1.67), 2.140 (1.24), 2.152 (1.49), 2.167 (2.28), 2.181 (1.18), 2.206 (1.00), 2.219 (1.77), 2.231 (1.77), 2.245 (0.92), 2.373 (0.84), 2.387 (0.71), 2.518 (2.93), 2.522 (2.91), 2.526 (2.32), 2.783 (1.43), 2.799 (2.49), 2.813 (2.87), 2.827 (1.35), 2.853 (3.24), 2.874 (3.83), 3.023 (3.24), 3.044 (3.14), 3.054 (0.69), 3.058 (0.71), 3.751 (7.72), 3.754 (7.87), 3.997 (0.80), 4.010 (1.39), 4.022 (2.85), 4.035 (3.91), 4.044 (2.47), 4.060 (1.26), 4.069 (1.22), 4.077 (4.91), 4.086 (5.24), 4.099 (1.79), 4.169 (0.71), 4.184 (0.77), 6.427 (1.75), 6.564 (8.60), 6.647 (16.00), 7.017 (13.80), 8.035 (5.14), 8.039 (5.24), 8.314 (1.94), 8.612 (4.73), 8.616 (4.65), 9.647 (0.75).

### Example 582

### 2-amino-5-{1-[(1H-imidazol-2-yl)methyl]-5′,6′-dihydrospiro[azetidine-3,4′-pyrrolo[1,2-b]pyrazol]-2′-yl}pyridine-3-carbonitrile

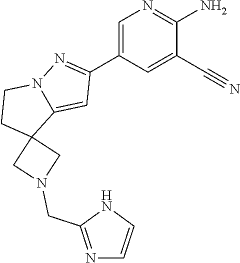 [SMILES: N#Cc1cc(-c2cc3n(n2)CCC32CN(Cc3ncc[nH]3)C2)cnc1N]

Example 582 was prepared in analogy to Example 566 using 2-amino-5-(5′,6′-dihydrospiro[azetidine-3,4′-pyrrolo[1,2-b]pyrazol]-2′-yl)pyridine-3-carbonitrile-hydrochloride salt (100 mg, 330 μmol) and 1H-imidazole-2-carbaldehyde (47.6 mg, 495 μmol).

1H NMR (400 MHz, DMSO-d6) δ 11.62-12.01 (m, 1H), 8.58-8.68 (m, 1H), 8.05-8.22 (m, 1H), 7.00-7.10 (m, 1H), 6.99 (s, 2H), 6.73-6.90 (m, 1H), 6.60 (s, 1H), 4.04-4.11 (m, 2H), 3.57-3.65 (m, 2H), 3.41-3.48 (m, 2H), 3.36-3.39 (m, 2H), 2.75-2.81 (m, 2H)

LC-MS (method 1): Rₜ=0.66 min; MS (ESIpos): m/z=347 [M+H]⁺

### Example 583

### 5-{(rac)-1-[(1-methyl-1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine

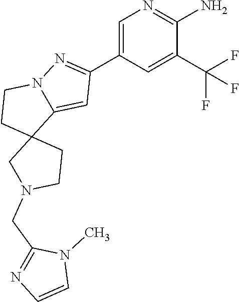 [SMILES: Cn1ccnc1CN1CCC2(CCn3nc(-c4cnc(N)c(C(F)(F)F)c4)cc32)C1]

Example 583 was prepared in analogy to Example 566 using 5-[(rac)-5′,6′-dihydrospiro[pyrrolidine-3,4′-pyrrolo[1,2-b]pyrazol]-2′-yl]-3-(trifluoromethyl)pyridin-2-amine-hydrochloride salt (100 mg, 278 μmol) and 1-methyl-1H-imidazole-2-carbaldehyde (45.9 mg, 417 μmol).

1H NMR (400 MHz, DMSO-d6) δ 8.53-8.62 (m, 1H), 7.90-8.05 (m, 1H), 6.98-7.12 (m, 1H), 6.67-6.80 (m, 1H), 6.48-6.60 (m, 2H), 6.33-6.47 (m, 1H), 4.04-4.16 (m, 2H), 3.69 (s, 5H), 2.51-2.75 (m, 6H), 1.92-2.11 (m, 2H)

LC-MS (method 1): Rₜ=0.94 min; MS (ESIpos): m/z=419 [M+H]⁺

### Example 584

### 5-{(rac)-1-[(1-methyl-1H-pyrazol-3-yl)methyl]-5′,6′-dihydrospiro[pyrrolidine-3,4′-pyrrolo[1,2-b]pyrazol]-2′-yl}-3-(trifluoromethyl)pyridin-2-amine

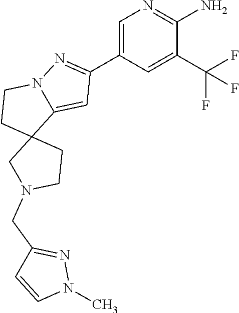 [SMILES: Cn1ccc(CN2CCC3(CCn4nc(-c5cnc(N)c(C(F)(F)F)c5)cc43)C2)n1]

Example 584 was prepared in analogy to Example 566 using 5-[(rac)-5′,6′-dihydrospiro[pyrrolidine-3,4′-pyrrolo[1,2-b]pyrazol]-2′-yl]-3-(trifluoromethyl)pyridin-2-amine-hydrochloride salt (100 mg, 278 μmol) and 1-methyl-1H-pyrazole-3-carbaldehyde (45.9 mg, 417 μmol).

1H NMR (400 MHz, DMSO-d6) δ 8.56-8.62 (m, 1H), 7.98-8.05 (m, 1H), 7.54-7.61 (m, 1H), 6.48-6.56 (m, 2H), 6.41-6.48 (m, 1H), 6.11-6.21 (m, 1H), 4.03-4.15 (m, 2H), 3.73-3.79 (m, 3H), 3.50-3.64 (m, 2H), 2.51-2.81 (m, 6H), 1.91-2.10 (m, 2H)

LC-MS (method 1): Rₜ=0.95 min; MS (ESIpos): m/z=419 [M+H]⁺

### Example 585

### 2-amino-5-{(rac)-1-[(1H-imidazol-2-yl)methyl]-5′,6′-dihydrospiro[pyrrolidine-3,4′-pyrrolo[1,2-b]pyrazol]-2′-yl}pyridine-3-carbonitrile

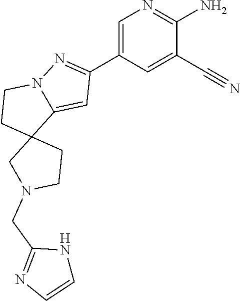 [SMILES: N#Cc1cc(-c2cc3n(n2)CCC32CCN(Cc3ncc[nH]3)C2)cnc1N]

Example 585 was prepared in analogy to Example 566 using 2-amino-5-[(rac)-5′,6′-dihydrospiro[pyrrolidine-3,4′-pyrrolo[1,2-b]pyrazol]-2′-yl]pyridine-3-carbonitrile-hydrochloride salt (98.0 mg, 309 μmol) and 1H-imidazole-2-carbaldehyde (44.6 mg, 464 μmol).

1H NMR (400 MHz, DMSO-d6) δ 11.34-11.97 (m, 1H), 8.45-8.82 (m, 1H), 7.76-8.30 (m, 1H), 7.00-7.08 (m, 1H), 6.94-7.00 (m, 2H), 6.74-6.86 (m, 1H), 6.44 (s, 1H), 4.02-4.18 (m, 2H), 3.67 (s, 2H), 2.54-2.81 (m, 5H), 1.94-2.07 (m, 2H)

LC-MS (method 1): Rₜ=0.70 min; MS (ESIpos): m/z=361 [M+H]⁺

### Example 586

### 5-{1-[(1-methyl-1H-imidazol-2-yl)methyl]-5′,6′-dihydrospiro[azetidine-3,4′-pyrrolo[1,2-b]pyrazol]-2′-yl}-3-(trifluoromethyl)pyridin-2-amine

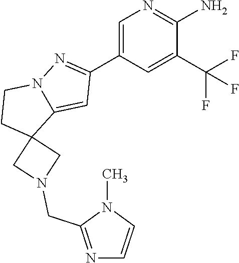 [SMILES: Cn1ccnc1CN1CC2(CCn3nc(-c4cnc(N)c(C(F)(F)F)c4)cc32)C1]

Example 586 was prepared in analogy to Example 566 using 5-(5′,6′-dihydrospiro[azetidine-3,4′-pyrrolo[1,2-b]pyrazol]-2′-yl)-3-(trifluoromethyl)pyridin-2-amine-hydrochloride salt (100 mg, 57% purity, 165 μmol) and 1-methyl-1H-imidazole-2-carbaldehyde (27.2 mg, 247 μmol).

1H NMR (400 MHz, DMSO-d6) δ 8.62 (d, 1H), 8.03 (d, 1H), 7.07 (d, 1H), 6.74 (d, 1H), 6.65 (s, 1H), 6.53 (s, 2H, NH2), 4.07 (t, 2H), 3.62-3.70 (m, 5H), 3.35-3.43 (m, 5H), 2.77 (t, 2H)

LC-MS (method 1): Rₜ=0.87 min; MS (ESIpos): m/z=405 [M+H]⁺

### Example 587

### 5-{(rac)-1-[(5-methyl-1H-pyrazol-3-yl)methyl]-5′,6′-dihydrospiro[pyrrolidine-3,4′-pyrrolo[1,2-b]pyrazol]-2′-yl}-3-(trifluoromethyl)pyridin-2-amine

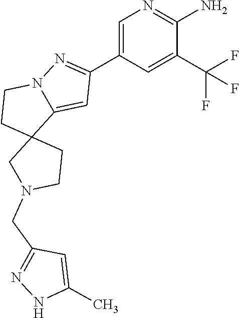 [SMILES: Cc1cc(CN2CCC3(CCn4nc(-c5cnc(N)c(C(F)(F)F)c5)cc43)C2)n[nH]1]

Example 587 was prepared in analogy to Example 566 using 5-[(rac)-5′,6′-dihydrospiro[pyrrolidine-3,4′-pyrrolo[1,2-b]pyrazol]-2′-yl]-3-(trifluoromethyl)pyridin-2-amine-hydrochloride salt (100 mg, 278 μmol) and 5-methyl-1H-pyrazole-3-carbaldehyde (45.9 mg, 417 μmol).

1H NMR (400 MHz, DMSO-d6) δ 11.79-12.56 (m, 1H), 8.25-8.82 (m, 1H), 7.77-8.18 (m, 1H), 6.48-6.56 (m, 2H), 6.42-6.46 (m, 1H), 5.86-5.94 (m, 1H), 4.03-4.16 (m, 2H), 3.51-3.64 (m, 2H), 2.51-2.80 (m, 6H), 2.11-2.22 (m, 3H), 1.94-2.08 (m, 2H)

### Example 588

### 5-{(rac)-1-[(2,4-dimethyl-1H-imidazol-5-yl)methyl]-5′,6′-dihydrospiro[pyrrolidine-3,4′-pyrrolo[1,2-b]pyrazol]-2′-yl}-3-(trifluoromethyl)pyridin-2-amine

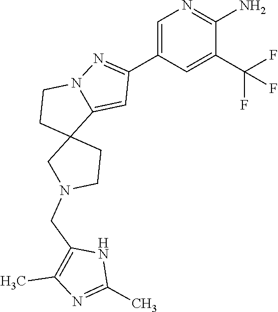 [SMILES: Cc1nc(C)c(CN2CCC3(CCn4nc(-c5cnc(N)c(C(F)(F)F)c5)cc43)C2)[nH]1]

Example 588 was prepared in analogy to Example 566 using 5-[(rac)-5′,6′-dihydrospiro[pyrrolidine-3,4′-pyrrolo[1,2-b]pyrazol]-2′-yl]-3-(trifluoromethyl)pyridin-2-amine-hydrochloride salt (100 mg, 278 μmol) and 2,4-dimethyl-1H-imidazole-5-carbaldehyde (51.8 mg, 417 μmol).

H-NMR-Data 1H NMR (400 MHz, DMSO-d6) δ 8.47-8.66 (m, 1H), 7.90-8.12 (m, 1H), 6.47-6.57 (m, 2H), 6.39-6.45 (m, 1H), 4.03-4.15 (m, 2H), 3.44-3.47 (m, 2H), 2.69-2.80 (m, 2H), 2.51-2.64 (m, 4H), 2.12-2.19 (m, 3H), 2.03-2.07 (m, 3H), 1.92-2.03 (m, 2H)

LC-MS (method 1): Rₜ=0.90 min; MS (ESIneg): m/z=430 [M−H]⁻

### Example 589

### 5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (mixture of stereoisomers)

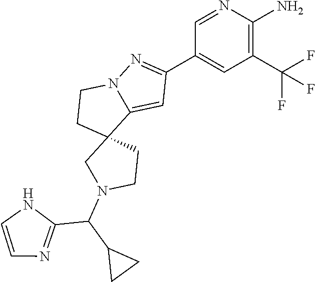 [SMILES: Nc1ncc(-c2cc3n(n2)CC[C@@]32CCN(C(c3ncc[nH]3)C3CC3)C2)cc1C(F)(F)F]

A solution of 5-[(3R)-5′,6′-dihydrospiro[pyrrolidine-3,4′-pyrrolo[1,2-b]pyrazol]-2′-yl]-3-(trifluoromethyl)pyridin-2-amine hydrogen chloride (125 mg, 79% purity, 274 μmol), cyclopropyl(1H-imidazol-2-yl)methanone (56.1 mg, 412 μmol), Titanium(IV) isopropoxide (240 μl, 820 μmol) and DIPEA (190 μl, 1.1 mmol) in MeOH (2.7 mL) was stirred at 60° C. for 2 h. To this sodium cyanoborohydride (43.1 mg, 686 μmol) was added and the mixture was stirred at RT overnight.

The mixture was filtered and the filtrate concentrated in vacuo. The crude was purified by basic HPLC to give 4.00 mg (90% purity, 3% yield) of the title compound.

H-NMR-Data—1H NMR (DMSO-d6, 400 MHz) δ 11.79 (br s, 1H), 8.59 (d, 1H), 8.00 (s, 1H), 7.0-7.1 (m, 1H), 6.75 (d, 1H), 6.52 (s, 2H), 6.44 (d, 1H), 4.0-4.2 (m, 2H), 2.9-3.0 (m, 1H), 2.7-2.8 (m, 1H), 2.7-2.7 (m, 1H), 2.6-2.6 (m, 2H), 1.9-2.1 (m, 2H), 1.23 (dt, 1H), 0.6-0.7 (m, 1H), 0.3-0.4 (m, 2H), 0.0-0.1 (m, 1H)

LC-MS (method 1): Rₜ=0.95 min; MS (ESIpos): m/z=445 [M+H]⁺

### Example 590

### 5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (Stereoisomer 1)

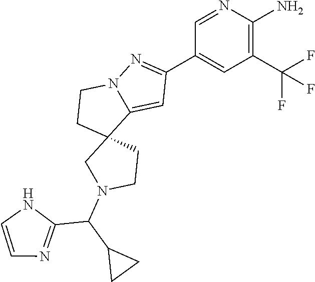 [SMILES: Nc1ncc(-c2cc3n(n2)CC[C@@]32CCN(C(c3ncc[nH]3)C3CC3)C2)cc1C(F)(F)F]

5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (296 mg, 667 μmol) was submitted to the HPLC-Lab for chiral separation. Enantiomer #1 was purified further by basic HPLC to give 62.0 mg (99% purity, 21% yield) of the title compound.

H-NMR-Data—1H NMR (DMSO-d6, 400 MHz) δ 11.79 (br s, 1H), 8.59 (d, 1H), 8.00 (d, 1H), 7.02 (br s, 1H), 6.75 (br s, 1H), 6.53 (s, 2H), 6.45 (s, 1H), 4.10 (t, 2H), 2.94 (d, 1H), 2.78 (dt, 1H), 2.66 (d, 1H), 2.5-2.6 (m, 3H), 1.9-2.1 (m, 2H), 1.2-1.3 (m, 1H), 0.6-0.7 (m, 1H), 0.3-0.4 (m, 2H), 0.0-0.1 (m, 1H)

[α]²⁰_{D}: +54.38° (c=1.00, DMSO)

LC-MS (method 1): Rₜ=0.96 min; MS (ESIpos): m/z=445 [M+H]⁺

### Example 591

### 5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (Stereoisomer 2)

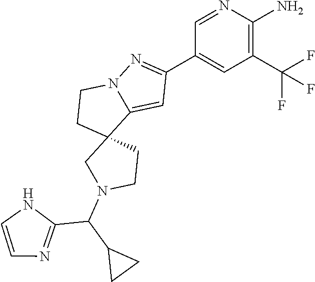 [SMILES: Nc1ncc(-c2cc3n(n2)CC[C@@]32CCN(C(c3ncc[nH]3)C3CC3)C2)cc1C(F)(F)F]

5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (296 mg, 667 μmol) was submitted to the HPLC-Lab for chiral separation. Enantiomer #2 was purified further by basic HPLC to give 54.0 mg (99% purity, 18% yield) of the title compound.

1H NMR (DMSO-d6, 400 MHz) δ 11.80 (br s, 1H), 8.59 (d, 1H), 8.00 (d, 1H), 7.03 (br s, 1H), 6.75 (br s, 1H), 6.52 (s, 2H), 6.44 (s, 1H), 4.0-4.2 (m, 2H), 2.7-2.8 (m, 2H), 2.5-2.6 (m, 3H), 1.9-2.0 (m, 2H), 1.1-1.3 (m, 1H), 0.5-0.7 (m, 1H), 0.2-0.4 (m, 2H), 0.08 (qd, 1H)

[α]²⁰_{D}: +49.51° (c=1.00, DMSO)

LC-MS (method 1): Rₜ=0.96 min; MS (ESIpos): m/z=445 [M+H]⁺

### Example 592

### 5-{(3R)-1-[(1-(1H-imidazol-2-yl)propyl]-5′,6′-dihydrospiro[pyrrolidine-3,4′-pyrrolo[1,2-b]pyrazol]-2′-yl}-3-(trifluoromethyl)pyridin-2-amine (Mixture of Stereoisomers)

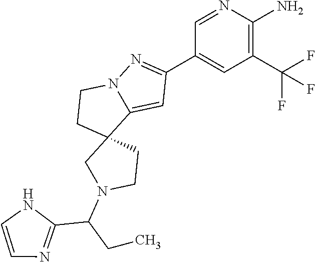 [SMILES: CCC(c1ncc[nH]1)N1CC[C@@]2(CCn3nc(-c4cnc(N)c(C(F)(F)F)c4)cc32)C1]

A solution of 5-[(3R)-5′,6′-dihydrospiro[pyrrolidine-3,4′-pyrrolo[1,2-b]pyrazol]-2′-yl]-3-(trifluoromethyl)pyridin-2-amine hydrogen chloride salt (125 mg, 79% purity, 274 μmol), 1-(1H-imidazol-2-yl)propan-1-one (51.1 mg, 412 μmol), Titanium(IV) isopropoxide (240 μl, 820 μmol) and DIPEA (190 μl, 1.1 mmol) in MeOH (2.7 mL) was stirred at 60° C. for 2 h. To this sodium cyanoborohydride (43.1 mg, 686 μmol) was added and the mixture was stirred at RT overnight. The resultant mixture was filtered and the filtrate concentrated in vacuo. The crude was purified by basic HPLC to give 33.0 mg (98% purity, 27% yield) of the title compound.

H-NMR-Data—1H NMR (DMSO-d6, 400 MHz) δ 11.78 (br s, 1H), 8.57 (t, 1H), 7.99 (t, 1H), 7.04 (dt, 1H), 6.8-6.9 (m, 1H), 6.52 (s, 2H), 6.31 (d, 1H), 4.0-4.1 (m, 2H), 3.52 (ddd, 1H), 2.7-2.8 (m, 1H), 2.5-2.7 (m, 3H), 2.4-2.5 (m, 1H), 1.7-2.0 (m, 4H), 0.73 (dt, 3H)

LC-MS (method 1): Rₜ=0.93 min; MS (ESIpos): m/z=433 [M+H]⁺

### Example 593

### 5-{(3R)-1-[1-(1H-imidazol-2-yl)propyl]-5′,6′-dihydrospiro[pyrrolidine-3,4′-pyrrolo[1,2-b]pyrazol]-2′-yl}-3-(trifluoromethyl)pyridin-2-amine (Stereoisomer 1)

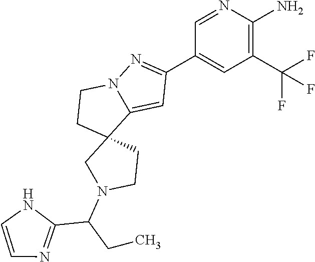 [SMILES: CCC(c1ncc[nH]1)N1CC[C@@]2(CCn3nc(-c4cnc(N)c(C(F)(F)F)c4)cc32)C1]

5-{(3R)-1-[1-(1H-imidazol-2-yl)propyl]-5′,6′-dihydrospiro[pyrrolidine-3,4′-pyrrolo[1,2-b]pyrazol]-2′-yl}-3-(trifluoromethyl)pyridin-2-amine (270 mg, 626 μmol) was submitted to the HPLC-Lab for chiral separation. Enantiomer #1 was purified further by basic HPLC to give 59.0 mg (95% purity, 21% yield) of the title compound.

H-NMR-Data—1H NMR (DMSO-d6, 400 MHz) δ 11.78 (br s, 1H), 8.5-8.7 (m, 1H), 7.99 (d, 1H), 7.04 (br s, 1H), 6.81 (br s, 1H), 6.52 (s, 2H), 6.34 (s, 1H), 4.08 (t, 2H), 3.52 (dd, 1H), 2.7-2.8 (m, 2H), 2.6-2.6 (m, 1H), 2.55 (d, 1H), 2.4-2.5 (m, 1H), 1.7-2.0 (m, 4H), 0.73 (t, 3H)

[α]²⁰_{D}: +35.53° (c=1.00, DMSO)

LC-MS (method 1): Rₜ=0.94 min; MS (ESIpos): m/z=433 [M+H]⁺

### Example 594

### 5-{(3R)-1-[1-(1H-imidazol-2-yl)propyl]-5′,6′-dihydrospiro[pyrrolidine-3,4′-pyrrolo[1,2-b]pyrazol]-2′-yl}-3-(trifluoromethyl)pyridin-2-amine (Stereoisomer 2)

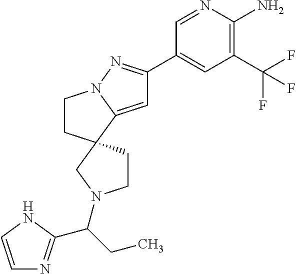 [SMILES: CCC(c1ncc[nH]1)N1CC[C@@]2(CCn3nc(-c4cnc(N)c(C(F)(F)F)c4)cc32)C1]

5-{(3R)-1-[1-(1H-imidazol-2-yl)propyl]-5′,6′-dihydrospiro[pyrrolidine-3,4′-pyrrolo[1,2-b]pyrazol]-2′-yl}-3-(trifluoromethyl)pyridin-2-amine (270 mg, 626 μmol) was submitted to the HPLC-Lab for chiral separation. Enantiomer #2 was purified further by basic HPLC to give 44.0 mg (95% purity, 15% yield) of the title compound.

H-NMR-Data—1H NMR (DMSO-d6, 400 MHz) δ 11.78 (br s, 1H), 8.57 (d, 1H), 7.99 (d, 1H), 7.04 (br s, 1H), 6.81 (br s, 1H), 6.52 (s, 2H), 6.29 (s, 1H), 4.0-4.2 (m, 2H), 3.53 (dd, 1H), 2.8-2.9 (m, 1H), 2.6-2.7 (m, 1H), 2.6-2.6 (m, 2H), 2.4-2.5 (m, 2H), 1.92 (t, 2H), 1.7-1.9 (m, 2H), 0.7-0.8 (m, 3H)

[α]²⁰_{D}: +53.59° (c=1.00, DMSO)

LC-MS (method 1): Rₜ=0.94 min; MS (ESIpos): m/z=433 [M+H]⁺

### Example 595

### 5-{(3R)-1-[1-(1-methyl-1H-imidazol-2-yl)ethyl]-5′,6′-dihydrospiro[pyrrolidine-3,4′-pyrrolo[1,2-b]pyrazol]-2′-yl}-3-(trifluoromethyl)pyridin-2-amine (Mixture of Stereoisomers)

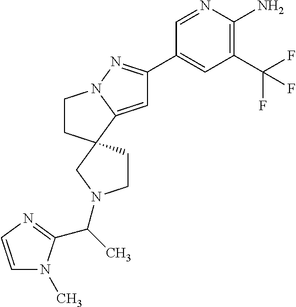 [SMILES: CC(c1nccn1C)N1CC[C@@]2(CCn3nc(-c4cnc(N)c(C(F)(F)F)c4)cc32)C1]

A mixture of 5-[(3R)-5′,6′-dihydrospiro[pyrrolidine-3,4′-pyrrolo[1,2-b]pyrazol]-2′-yl]-3-(trifluoromethyl)pyridin-2-amine hydrogen chloride (800 mg, 79% purity, 1.76 mmol), 1-methyl-1H-imidazole-2-carbaldehyde (290 mg, 2.63 mmol) and Na2SO4 in THF under Argon was stirred at RT overnight. The mixture was cooled to 0° C. and treated with methyl magnesium chloride (2.3 ml, 3.0 M, 7.0 mmol) and continued stirring at 0° C. for 1 h. The mixture was quenched with 2 mL of std. NH4CI-solution, the mixture was filtered and concentrated in vacuo. The crude was pre-purified by column chromatography with an isocratic gradient of Ethyl acetate followed by 0-20% methanol in DCM. The obtained product (186 mg) was further purified by basic HPLC to give 24.0 mg (99% purity, 3% yield) of the title compound.

H-NMR-Data—1H NMR (DMSO-d6, 400 MHz) δ 8.58 (t, 1H), 8.00 (t, 1H), 7.02 (dd, 1H), 6.73 (dd, 1H), 6.52 (s, 2H), 6.40 (d, 1H), 4.09 (t, 2H), 3.94 (qd, 1H), 3.73 (d, 3H), 2.79 (dt, 1H), 2.7-2.7 (m, 1H), 2.5-2.6 (m, 3H), 1.9-2.1 (m, 2H), 1.37 (dd, 3H)

LC-MS (method 1): Rₜ=0.99 min; MS (ESIpos): m/z=433 [M+H]⁺

### Example 596

### 3-(trifluoromethyl)-5-[(3R)-1-{[4-(trifluoromethyl)-1H-imidazol-2-yl]methyl}-5′,6′-dihydrospiro[pyrrolidine-3,4′-pyrrolo[1,2-b]pyrazol]-2′-yl]pyridin-2-amine

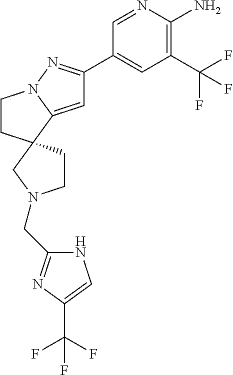 [SMILES: Nc1ncc(-c2cc3n(n2)CC[C@@]32CCN(Cc3nc(C(F)(F)F)c[nH]3)C2)cc1C(F)(F)F]

To a mixture of 5-[(3R)-5′,6′-dihydrospiro[pyrrolidine-3,4′-pyrrolo[1,2-b]pyrazol]-2′-yl]-3-(trifluoromethyl)pyridin-2-amine hydrogen chloride (125 mg, 79% purity, 274 μmol) and 4-(trifluoromethyl)-1H-imidazole-2-carbaldehyde (67.6 mg, 412 μmol) in THF (2.7 mL), acetic acid (24 μl) was added and stirred for 30 min. To this sodium triacetoxyborohydride (116 mg, 549 μmol) was added and the mixture was stirred at RT overnight. The mixture was diluted with water and then extracted with Ethyl acetate (2×). The combined organics were washed with brine, dried over sodium sulfate and concentrated in vacuo. The crude was purified by basic to give 7.00 mg (98% purity, 5% yield) of the title compound.

H-NMR-Data—1H NMR (DMSO-d6, 400 MHz) δ 12.59 (br s, 1H), 8.58 (d, 1H), 8.00 (d, 1H), 7.70 (s, 1H), 6.53 (s, 2H), 6.47 (s, 1H), 4.10 (t, 2H), 3.73 (s, 2H), 2.7-2.8 (m, 2H), 2.6-2.7 (m, 2H), 2.6-2.6 (m, 1H), 1.9-2.1 (m, 2H)

LC-MS (method 1): Rₜ=1.02 min; MS (ESIpos): m/z=472 [M+H]⁺

### Example 597

### 2-amino-5-[(rac)-1-(ethylcarbamoyl)-5′,6′-dihydrospiro[pyrrolidine-3,4′-pyrrolo[1,2-b]pyrazol]-2′-yl]pyridine-3-carboxylic acid

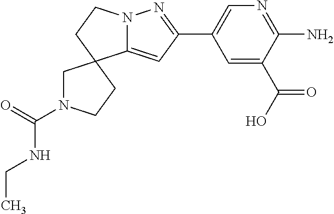 [SMILES: CCNC(=O)N1CCC2(CCn3nc(-c4cnc(N)c(C(=O)O)c4)cc32)C1]

LiOH (115 mg, 4.81 mmol) was stirred in water (8.3 mL) for 15 min at rt. methyl 2-amino-5-[(rac)-1-(ethylcarbamoyl)-5′,6′-dihydrospiro[pyrrolidine-3,4′-pyrrolo[1,2-b]pyrazol]-2′-yl]pyridine-3-carboxylate (100 mg, 260 μmol) was solubilised in methanol (8.3 mL) and added to the mixture. It was stirred overnight at rt. The mixture was evaporated and the residue was purified by preparative HPLC to give 18.0 mg (95% purity, 18% yield) of the title compound.

LC-MS (Method 1): Rt=0.34 min; MS (ESIpos): m/z=371 [M+H]⁺

¹H-NMR (500 MHz, DMSO-d6) δ [ppm]: 1.010 (1.78), 1.024 (3.54), 1.039 (1.54), 1.043 (0.88), 1.057 (1.66), 1.071 (0.83), 2.079 (0.38), 2.522 (0.78), 2.525 (0.75), 2.528 (0.69), 2.543 (16.00), 3.045 (0.63), 3.056 (0.71), 3.059 (0.66), 3.070 (0.61), 3.352 (1.07), 3.365 (1.10), 3.371 (0.95), 3.380 (0.81), 3.386 (0.94), 3.406 (2.58), 3.420 (0.48), 3.434 (0.70), 3.448 (0.64), 4.152 (0.58), 4.167 (1.00), 4.180 (0.56), 6.148 (0.48), 6.295 (1.39), 8.315 (0.34), 8.323 (0.72), 8.328 (0.86), 8.381 (0.42).

### Example 598

### 5-[(3S)-1-(pyridin-2-ylmethyl)-5′,6′-dihydrospiro[pyrrolidine-3,4′-pyrrolo[1,2-b]pyrazol]-2′-yl]-3-(trifluoromethyl)pyridin-2-amine

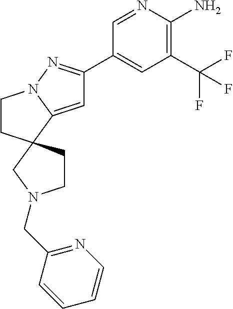 [SMILES: Nc1ncc(-c2cc3n(n2)CC[C@]32CCN(Cc3ccccn3)C2)cc1C(F)(F)F]

5-[(3S)-5′,6′-dihydrospiro[pyrrolidine-3,4′-pyrrolo[1,2-b]pyrazol]-2′-yl]-3-(trifluoromethyl)pyridin-2-amine-hydrochloride salt (100 mg, 84% purity, 233 μmol), 2-(chloromethyl)pyridine (35.7 mg, 280 μmol) and K2CO3 (129 mg, 934 μmol) were stirred in THF (2.5 mL) overnight at rt. KI (38.8 mg, 233 μmol) was added and the mixture was stirred for 2 h at 60° C. It was diluted with water and extracted 2× with ethyl acetate. The combined organic layers were washed with brine, dried over Na₂SO₄ and evaporated. The residue was purified by preparative HPLC to give 25.0 mg (99% purity, 26% yield) of the title compound.

1H NMR (DMSO-d6, 400 MHz) δ 8.60 (d, 1H), 8.5-8.5 (m, 1H), 8.02 (d, 1H), 7.77 (dt, 1H), 7.50 (d, 1H), 7.25 (ddd, 1H), 6.5-6.5 (m, 2H), 6.49 (s, 1H), 4.1-4.2 (m, 2H), 3.7-3.8 (m, 2H), 2.7-2.8 (m, 4H), 2.5-2.6 (m, 2H), 2.0-2.1 (m, 2H)

[α]²⁰_{D}: −62.2° (c=1.00, DMSO)

LC-MS (method 1): Rₜ=1.03 min; MS (ESIpos): m/z=415 [M+H]⁺

### Example 599

### 5-[(3R)-1-(pyridin-2-ylmethyl)-5′,6′-dihydrospiro[pyrrolidine-3,4′-pyrrolo[1,2-b]pyrazol]-2′-yl]-3-(trifluoromethyl)pyridin-2-amine

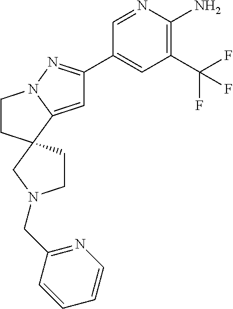 [SMILES: Nc1ncc(-c2cc3n(n2)CC[C@@]32CCN(Cc3ccccn3)C2)cc1C(F)(F)F]

Example 599 was prepared in analogy to Example 598 using 5-[(3R)-5′,6′-dihydrospiro[pyrrolidine-3,4′-pyrrolo[1,2-b]pyrazol]-2′-yl]-3-(trifluoromethyl)pyridin-2-amine-hydrochloride salt (100 mg, 82% purity, 228 μmol, stereoisomer 2) and 2-(chloromethyl)pyridine (34.9 mg, 273 μmol).

1H NMR (DMSO-d6, 400 MHz) δ 8.6-8.7 (m, 1H), 8.4-8.6 (m, 1H), 7.9-8.1 (m, 1H), 7.7-7.8 (m, 1H), 7.4-7.6 (m, 1H), 7.2-7.4 (m, 1H), 6.5-6.6 (m, 2H), 6.49 (s, 1H), 4.1-4.2 (m, 2H), 3.7-3.9 (m, 2H), 2.7-2.9 (m, 4H), 2.5-2.7 (m, 2H), 2.0-2.1 (m, 2H)

[α]²⁰_{D}: +65.3° (c=1.00, DMSO)

LC-MS (method 1): Rₜ=1.03 min; MS (ESIpos): m/z=415 [M+H]⁺

### Example 600

### 5-[(3S)-1-(pyridin-4-ylmethyl)-5′,6′-dihydrospiro[pyrrolidine-3,4′-pyrrolo[1,2-b]pyrazol]-2′-yl]-3-(trifluoromethyl)pyridin-2-amine

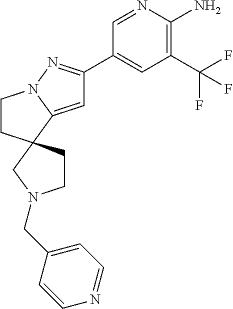 [SMILES: Nc1ncc(-c2cc3n(n2)CC[C@]32CCN(Cc3ccncc3)C2)cc1C(F)(F)F]

Example 600 was prepared in analogy to Example 598 using 5-[(3S)-5′,6′-dihydrospiro[pyrrolidine-3,4′-pyrrolo[1,2-b]pyrazol]-2′-yl]-3-(trifluoromethyl)pyridin-2-amine-hydrochloride salt (100 mg, 84% purity, 233 μmol) and 4-(chloromethyl)pyridine-hydrochloride salt (46.0 mg, 280 μmol).

1H NMR (DMSO-d6, 400 MHz) δ 8.6-8.7 (m, 1H), 8.4-8.6 (m, 2H), 7.8-8.2 (m, 1H), 7.3-7.5 (m, 2H), 6.5-6.6 (m, 2H), 6.5-6.5 (m, 1H), 4.0-4.2 (m, 2H), 3.6-3.8 (m, 2H), 2.5-2.8 (m, 6H), 2.0-2.2 (m, 2H)

[α]²⁰_{D}: −66.6° (c=1.00, DMSO)

LC-MS (method 1): Rₜ=1.02 min; MS (ESIpos): m/z=415 [M+H]⁺

### Example 601

### 5-[(3R)-1-(pyridin-4-ylmethyl)-5′,6′-dihydrospiro[pyrrolidine-3,4′-pyrrolo[1,2-b]pyrazol]-2′-yl]-3-(trifluoromethyl)pyridin-2-amine

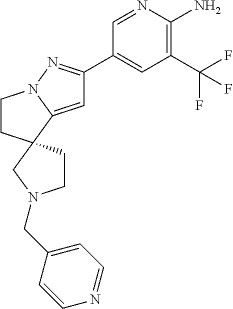 [SMILES: Nc1ncc(-c2cc3n(n2)CC[C@@]32CCN(Cc3ccncc3)C2)cc1C(F)(F)F]

Example 601 was prepared in analogy to Example 598 using 5-[(3R)-5′,6′-dihydrospiro[pyrrolidine-3,4′-pyrrolo[1,2-b]pyrazol]-2′-yl]-3-(trifluoromethyl)pyridin-2-amine-hydrochloride salt (100 mg, 82% purity, 228 μmol) and 4-(chloromethyl)pyridine-hydrochloride salt (44.9 mg, 273 μmol).

1H NMR (DMSO-d6, 400 MHz) δ 8.6-8.6 (m, 1H), 8.4-8.6 (m, 2H), 7.9-8.0 (m, 1H), 7.3-7.5 (m, 2H), 6.5-6.6 (m, 2H), 6.5-6.5 (m, 1H), 4.0-4.2 (m, 2H), 3.6-3.8 (m, 2H), 2.5-2.8 (m, 6H), 2.0-2.1 (m, 2H)

[α]²⁰_{D}: +67.4° (c=1.00, DMSO)

LC-MS (method 1): Rₜ=1.02 min; MS (ESIpos): m/z=415 [M+H]⁺

### Example 602

### 5-{(3R)-1-[3-(dimethylamino)propyl]-5′,6′-dihydrospiro[pyrrolidine-3,4′-pyrrolo[1,2-b]pyrazol]-2′-yl}-3-(trifluoromethyl)pyridin-2-amine

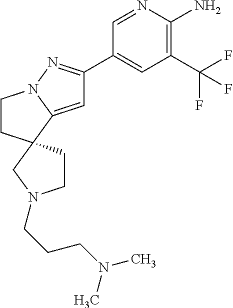 [SMILES: CN(C)CCCN1CC[C@@]2(CCn3nc(-c4cnc(N)c(C(F)(F)F)c4)cc32)C1]

5-[(3R)-5′,6′-dihydrospiro[pyrrolidine-3,4′-pyrrolo[1,2-b]pyrazol]-2′-yl]-3-(trifluoromethyl)pyridin-2-amine-hydrochloride salt (125 mg, 79% purity, 274 μmol), 3-chloro-N,N-dimethylpropan-1-amine-hydrochloride salt (52.1 mg, 329 μmol) and K2CO3 (152 mg, 1.10 mmol) were stirred in THF (3.0 mL) overnight at rt. NaI (82.2 mg, 548 μmol) was added and the mixture was stirred for 3 h at 70° C. It was diluted with water and extracted 2× with ethyl acetate. The combined organic layers were washed with brine, dried over Na2SO4 and evaporated. The residue was purified by preparative HPLC to give 7.00 mg (98% purity, 6% yield) of the title compound.

1H NMR (DMSO-d6, 400 MHz) δ 8.59 (d, 1H), 8.01 (d, 1H), 6.51 (s, 2H), 6.43 (s, 1H), 4.10 (t, 2H), 2.7-2.8 (m, 2H), 2.5-2.6 (m, 3H), 2.4-2.5 (m, 2H), 2.23 (t, 2H), 2.10 (s, 6H), 1.9-2.1 (m, 2H), 1.56 (quin, 2H)

LC-MS (method 1): Rₜ=1.05 min; MS (ESIpos): m/z=410 [M+H]⁺

### Example 603

### 5-{(3R)-1-[2-(dimethylamino)ethyl]-5′,6′-dihydrospiro[pyrrolidine-3,4′-pyrrolo[1,2-b]pyrazol]-2′-yl}-3-(trifluoromethyl)pyridin-2-amine

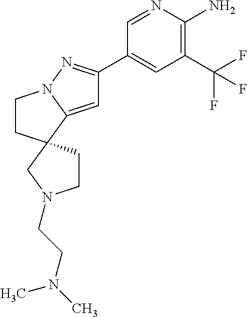 [SMILES: CN(C)CCN1CC[C@@]2(CCn3nc(-c4cnc(N)c(C(F)(F)F)c4)cc32)C1]

Example 603 was prepared in analogy to Example 602 using 5-[(3R)-5′,6′-dihydrospiro[pyrrolidine-3,4′-pyrrolo[1,2-b]pyrazol]-2′-yl]-3-(trifluoromethyl)pyridin-2-amine-hydrochloride salt (125 mg, 79% purity, 274 μmol) and 2-chloro-N,N-dimethylethan-1-amine-hydrochloride salt (47.4 mg, 329 μmol).

1H NMR (DMSO-d6, 400 MHz) δ 8.59 (d, 1H), 8.01 (d, 1H), 6.51 (s, 2H), 6.44 (s, 1H), 4.1-4.2 (m, 2H), 2.7-2.8 (m, 2H), 2.5-2.6 (m, 5H), 2.3-2.4 (m, 2H), 2.15 (s, 6H), 1.9-2.1 (m, 2H)

LC-MS (method 1): Rₜ=0.99 min; MS (ESIpos): m/z=396 [M+H]⁺

### Example 604

2-amino-1-{2′-[6-amino-5-(trifluoromethyl)pyridin-3-yl]-5′,6′-dihydrospiro[pyrrolidine-3,4′-pyrrolo[1,2-b]pyrazol]-1-yl}ethan-1-one (Stereoisomer 2)

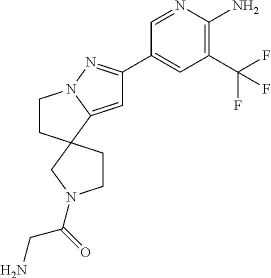 [SMILES: NCC(=O)N1CCC2(CCn3nc(-c4cnc(N)c(C(F)(F)F)c4)cc32)C1]

tert-butyl (2-{2′-[6-amino-5-(trifluoromethyl)pyridin-3-yl]-5′,6′-dihydrospiro[pyrrolidine-3,4′-pyrrolo[1,2-b]pyrazol]-1-yl}-2-oxoethyl)carbamate (60.0 mg, 125 μmol, stereoisomer 2) was solubilised in 1,4-dioxane (600 μL), HCl (620 μL, 4.0 M in 1,4-dioxane, 2.5 mmol) was added and the mixture was stirred overnight at rt. It was evaporated and the residue was purified by preparative HPLC to give 7.00 mg (98% purity, 14% yield) of the title compound.

1H NMR (DMSO-d6, 400 MHz) δ 8.5-8.6 (m, 1H), 7.9-8.2 (m, 1H), 6.5-6.6 (m, 2H), 6.5-6.5 (m, 1H), 4.1-4.3 (m, 2H), 3.51 (s, 4H), 3.2-3.3 (m, 2H), 2.5-2.6 (m, 2H), 2.1-2.2 (m, 1H), 2.0-2.1 (m, 1H)

[α]²⁰_{D}: +80.0° (c=1.00, DMSO)

LC-MS (method 1): Rₜ=0.75 min; MS (ESIpos): m/z=381 [M+H]⁺

### Example 605

### 2-amino-1-{2′-[6-amino-5-(trifluoromethyl)pyridin-3-yl]-5′,6′-dihydrospiro[pyrrolidine-3,4′-pyrrolo[1,2-b]pyrazol]-1-yl}ethan-1-one (Stereoisomer 1)

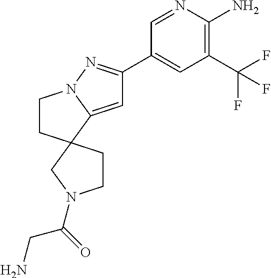 [SMILES: NCC(=O)N1CCC2(CCn3nc(-c4cnc(N)c(C(F)(F)F)c4)cc32)C1]

Example 605 was prepared in analogy to Example 604 using tert-butyl (2-{2′-[6-amino-5-(trifluoromethyl)pyridin-3-yl]-5′,6′-dihydrospiro[pyrrolidine-3,4′-pyrrolo[1,2-b]pyrazol]-1-yl}-2-oxoethyl)carbamate (96.0 mg, 200 μmol, stereoisomer 1).

1H NMR (DMSO-d6, 400 MHz) δ 8.5-8.7 (m, 1H), 7.9-8.1 (m, 1H), 6.5-6.6 (m, 2H), 6.5-6.5 (m, 1H), 4.1-4.3 (m, 2H), 3.5-3.7 (m, 4H), 3.2-3.3 (m, 2H), 2.5-2.6 (m, 2H), 2.1-2.2 (m, 1H), 2.0-2.1 (m, 1H)

[α]²⁰_{D}: −73.6° (c=1.00, DMSO)

LC-MS (method 1): Rₜ=0.75 min; MS (ESIpos): m/z=381 [M+H]⁺

### Example 606

### 2-amino-1-{2-[6-amino-5-(trifluoromethyl)pyridin-3-yl]-6,7-dihydrospiro[pyrazolo[5,1-c][1,4]oxazine-4,3′-pyrrolidin]-1′-yl}ethan-1-one (Stereoisomer 2)

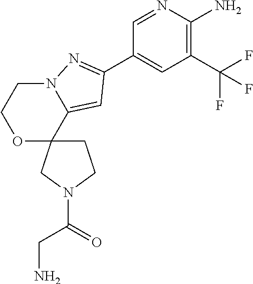 [SMILES: NCC(=O)N1CCC2(C1)OCCn1nc(-c3cnc(N)c(C(F)(F)F)c3)cc12]

Example 606 was prepared in analogy to Example 604 using tert-butyl (2-{2-[6-amino-5-(trifluoromethyl)pyridin-3-yl]-6,7-dihydrospiro[pyrazolo[5,1-c][1,4]oxazine-4,3′-pyrrolidin]-1′-yl}-2-oxoethyl)carbamate (163 mg, 328 μmol, stereoisomer 2).

1H NMR (DMSO-d6, 400 MHz) δ 8.5-8.7 (m, 1H), 8.0-8.1 (m, 1H), 6.7-6.8 (m, 1H), 6.5-6.7 (m, 2H), 4.14 (s, 4H), 3.9-3.9 (m, 1H), 3.7-3.8 (m, 1H), 3.5-3.6 (m, 1H), 3.4-3.5 (m, 1H), 3.1-3.3 (m, 2H), 2.2-2.5 (m, 2H)

[α]²⁰_{D}: +71.5° (c=1.00, DMSO)

LC-MS (method 1): Rₜ=0.75 min; MS (ESIpos): m/z=397 [M+H]⁺

### Example 607

### 2-amino-1-{2-[6-amino-5-(trifluoromethyl)pyridin-3-yl]-6,7-dihydrospiro[pyrazolo[5,1-c][1,4]oxazine-4,3′-pyrrolidin]-1′-yl}ethan-1-one (Stereoisomer 1)

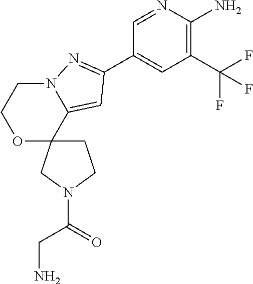 [SMILES: NCC(=O)N1CCC2(C1)OCCn1nc(-c3cnc(N)c(C(F)(F)F)c3)cc12]

Example 607 was prepared in analogy to Example 604 using tert-butyl (2-{2-[6-amino-5-(trifluoromethyl)pyridin-3-yl]-6,7-dihydrospiro[pyrazolo[5,1-c][1,4]oxazine-4,3′-pyrrolidin]-1′-yl}-2-oxoethyl)carbamate (163 mg, 328 μmol, stereoisomer 1).

1H NMR (DMSO-d6, 400 MHz) δ 8.5-8.6 (m, 1H), 8.0-8.0 (m, 1H), 6.7-6.8 (m, 1H), 6.5-6.7 (m, 2H), 4.14 (s, 4H), 3.8-4.0 (m, 1H), 3.6-3.8 (m, 1H), 3.5-3.6 (m, 1H), 3.4-3.5 (m, 1H), 3.1-3.3 (m, 2H), 2.2-2.5 (m, 2H)

[α]²⁰_{D}: −68.6° (c=1.00, DMSO)

LC-MS (method 1): Rₜ=0.75 min; MS (ESIpos): m/z=398 [M+H]⁺

### Example 608

### 2-amino-1-{2′-[6-amino-5-(trifluoromethyl)pyridin-3-yl]-5′,6′-dihydrospiro[azetidine-3,4′-pyrrolo[1,2-b]pyrazol]-1-yl}ethan-1-one

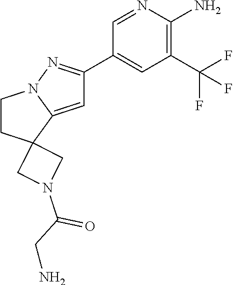 [SMILES: NCC(=O)N1CC2(CCn3nc(-c4cnc(N)c(C(F)(F)F)c4)cc32)C1]

Example 608 was prepared in analogy to Example 604 using tert-butyl (2-{2′-[6-amino-5-(trifluoromethyl)pyridin-3-yl]-5′,6′-dihydrospiro[azetidine-3,4′-pyrrolo[1,2-b]pyrazol]-1-yl}-2-oxoethyl)carbamate (70.0 mg, 150 μmol).

1H NMR (DMSO-d6) δ: 8.61 (d, 1H), 8.02 (d, 1H), 6.73 (s, 1H), 6.56 (s, 2H), 4.29-4.36 (m, 2H), 4.04-4.18 (m, 4H), 3.14 (s, 2H), 2.87 (m, 2H)

LC-MS (method 1): Rₜ=0.73 min; MS (ESIpos): m/z=368 [M+H]⁺

### Example 609

### (2S)-2-amino-1-{2′-[6-amino-5-(trifluoromethyl)pyridin-3-yl]-5′,6′-dihydrospiro[azetidine-3,4′-pyrrolo[1,2-b]pyrazol]-1-yl}propan-1-one

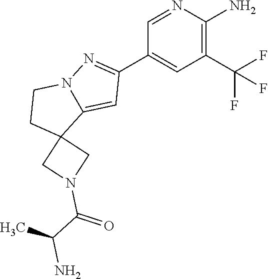 [SMILES: C[C@H](N)C(=O)N1CC2(CCn3nc(-c4cnc(N)c(C(F)(F)F)c4)cc32)C1]

Example 609 was prepared in analogy to Example 604 using tert-butyl [(2S)-1-{2′-[6-amino-5-(trifluoromethyl)pyridin-3-yl]-5′,6′-dihydrospiro[azetidine-3,4′-pyrrolo[1,2-b]pyrazol]-1-yl}-1-oxopropan-2-yl]carbamate (70.0 mg, 146 μmol).

1H NMR (DMSO-d6) δ: 8.61 (br s, 1H), 8.03 (m, 1H), 6.69-6.80 (m, 1H), 6.56 (s, 2H), 4.30-4.55 (m, 2H), 4.00-4.19 (m, 4H), 3.43 (br m, 1H), 2.86-2.95 (m, 2H), 1.11 (m, 3H)

LC-MS (method 1): Rₜ=0.76 min; MS (ESIpos): m/z=382 [M+H]⁺

### Example 610

### (2R)-2-amino-1-{2′-[6-amino-5-(trifluoromethyl)pyridin-3-yl]-5′,6′-dihydrospiro[azetidine-3,4′-pyrrolo[1,2-b]pyrazol]-1-yl}propan-1-one

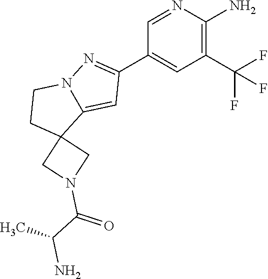 [SMILES: C[C@@H](N)C(=O)N1CC2(CCn3nc(-c4cnc(N)c(C(F)(F)F)c4)cc32)C1]

Example 610 was prepared in analogy to Example 604 using tert-butyl [(2R)-1-{2′-[6-amino-5-(trifluoromethyl)pyridin-3-yl]-5′,6′-dihydrospiro[azetidine-3,4′-pyrrolo[1,2-b]pyrazol]-1-yl}-1-oxopropan-2-yl]carbamate (70.0 mg, 146 μmol).

1H NMR (DMSO-d6) δ: 8.61 (br s, 1H), 7.97-8.08 (m, 1H), 6.74 (d, 1H), 6.56 (s, 2H), 4.47 (m, 1H), 4.36 (m, 1H), 3.99-4.20 (m, 4H), 3.38 (br m, 2H), 2.83-2.93 (m, 2H), 1.10 (m, 3H)

LC-MS (method 1): Rₜ=0.76 min; MS (ESIpos): m/z=382 [M+H]⁺

### Example 611

### (rac)-2′-[6-amino-5-(1-methylpiperidin-4-yl)pyridin-3-yl]-N-ethyl-5′,6′-dihydrospiro[pyrrolidine-3,4′-pyrrolo[1,2-b]pyrazole]-1-carboxamide

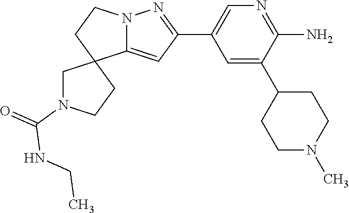

(rac)-2′-(2-amino-1′-methyl-1′,2′,3′,6′-tetrahydro[3,4′-bipyridin]-5-yl)-N-ethyl-5′,6′-dihydrospiro[pyrrolidine-3,4′-pyrrolo[1,2-b]pyrazole]-1-carboxamide (39.0 mg, 92.5 μmol) was solubilised in ethanol (2.0 mL), sparged with argon and Pd/C (4.92 mg, 10% purity, 4.63 μmol) was added. The flask was purged 3× with hydrogen and it was stirred over the weekend at rt. The mixture was filtered over Celite and the filtrate was evaporated. The residue was purified by preparative TLC to give 27.6 mg (93% purity, 66% yield) of the title compound.

1H-NMR (400 MHz, CHLOROFORM-d): δ [ppm]=1.17 (t, 3H), 1.90 (br s, 4H), 2.07 (br d, 3H), 2.23 (s, 1H), 2.33 (s, 4H), 2.57 (s, 1H), 2.63 (s, 1H), 3.01 (br d, 2H), 3.27-3.36 (m, 2H), 3.48-3.59 (m, 4H), 3.60-3.68 (m, 1H), 4.17 (br t, 1H), 4.26 (t, 2H), 4.49 (s, 2H), 6.13-6.16 (m, 1H), 7.76 (d, 1H), 8.33 (d, 1H).

LC-MS (method 1): Rₜ=0.80 min; MS (ESIpos): m/z=425 [M+H]⁺

### Example 612

### 5-{(rac)-1-[(5-methyl-1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine

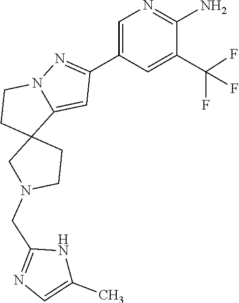 [SMILES: Cc1cnc(CN2CCC3(CCn4nc(-c5cnc(N)c(C(F)(F)F)c5)cc43)C2)[nH]1]

5-[(rac)-5′,6′-dihydrospiro[pyrrolidine-3,4′-pyrrolo[1,2-b]pyrazol]-2′-yl]-3-(trifluoromethyl)pyridin-2-amine-hydrochloride salt (100 mg, 278 μmol) and 2-(chloromethyl)-5-methyl-1H-imidazole-hydrochloride salt (55.7 mg, 334 μmol) were solubilized in THF (3.0 mL), K2CO3 (154 mg, 1.11 mmol) was added and the mixture was stirred for 1 h at rt. The mixture was diluted with water and extracted 2× with EtOAc. The combined organic layers were washed with brine, dried over Na₂SO₄ and evaporated. The residue was purified by preparative HPLC to give 16.0 mg (99% purity, 14% yield) of the title compound.

1H NMR (400 MHz, DMSO-d6) δ 10.96-12.10 (m, 1H), 8.47-8.66 (m, 1H), 7.93-8.05 (m, 1H), 6.56-6.62 (m, 1H), 6.50-6.56 (m, 2H), 6.38-6.49 (m, 1H), 4.02-4.16 (m, 2H), 3.55-3.66 (m, 2H), 2.72-2.81 (m, 2H), 2.65 (s, 2H), 2.54-2.62 (m, 1H), 2.06-2.11 (m, 3H), 1.95-2.05 (m, 2H)

LC-MS (method 1): Rₜ=0.88 min; MS (ESIneg): m/z=416 [M−H]⁻

### Example 613

### 2′-{6-amino-5-[(1R)-1-(2,6-dichloro-3-fluorophenyl)ethoxy]pyridin-3-yl}-N-ethyl-5′,6′-dihydrospiro[pyrrolidine-3,4′-pyrrolo[1,2-b]pyrazole]-1-carboxamide (Isomer 1)

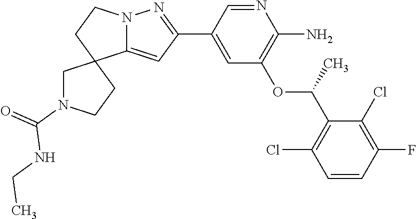 [SMILES: CCNC(=O)N1CCC2(CCn3nc(-c4cnc(N)c(O[C@H](C)c5c(Cl)ccc(F)c5Cl)c4)cc32)C1]

The mixture of isomers was separated by chiral HPLC to give 11.0 mg (98% purity, 3% yield) of the title compound.

Instrument: PrepCon Labomatic HPLC-4; Column: YMC Amylose SA 5μ, 250×30; eluent A: methyl tert-butyl ether+0.1 vol % diethylamine; eluent B: methanol; isocratic: 70% A+30% B; flow: 50 mL/min; temperature: 25° C.; UV: 254 nm;

Retention time: 3.5-5.6 min

Instrument: Thermo Fisher UltiMate 3000; Column: YMC Amylose SA 3μ, 100×4.6; eluent A: methyl tert-butyl ether+0.1 vol % diethylamine; eluent B: methanol; isocratic: 70% A+30% B; flow: 1.4 mL/min; temperature: 25° C.; UV: 254 nm

¹H-NMR (400 MHz, DMSO-d6) δ [ppm]: 1.002 (6.93), 1.020 (16.00), 1.038 (7.09), 1.232 (1.83), 1.775 (10.15), 1.792 (10.26), 2.009 (1.46), 2.028 (2.62), 2.040 (1.21), 2.046 (1.17), 2.469 (2.54), 2.518 (4.74), 2.522 (3.02), 3.022 (0.98), 3.039 (2.66), 3.053 (2.87), 3.057 (2.85), 3.071 (2.60), 3.089 (0.75), 3.359 (1.58), 3.375 (9.26), 3.384 (2.52), 3.402 (0.89), 3.437 (0.69), 3.454 (1.27), 3.462 (0.73), 3.469 (0.85), 3.479 (0.77), 4.108 (2.37), 4.126 (3.66), 4.143 (2.29), 5.785 (5.43), 6.037 (2.25), 6.053 (2.29), 6.130 (10.57), 6.138 (1.31), 6.152 (2.14), 6.166 (1.04), 7.074 (3.89), 7.079 (3.97), 7.419 (2.27), 7.442 (4.20), 7.463 (3.12), 7.546 (2.00), 7.559 (2.14), 7.569 (1.62), 7.581 (1.50), 7.879 (5.35), 7.883 (5.39).

### Example 614

### 2′-{6-amino-5-[(1R)-1-(2,6-dichloro-3-fluorophenyl)ethoxy]pyridin-3-yl}-N-ethyl-5′,6′-dihydrospiro[pyrrolidine-3,4′-pyrrolo[1,2-b]pyrazole]-1-carboxamide (Isomer 2)

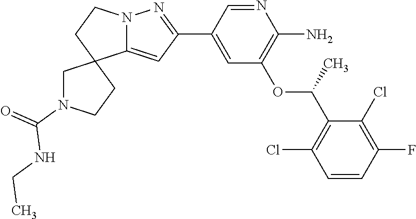 [SMILES: CCNC(=O)N1CCC2(CCn3nc(-c4cnc(N)c(O[C@H](C)c5c(Cl)ccc(F)c5Cl)c4)cc32)C1]

The mixture of isomers was separated by chiral HPLC to give 14.3 mg (98% purity, 3% yield) of the title compound.

Instrument: PrepCon Labomatic HPLC-4; Column: YMC Amylose SA 5μ, 250×30; eluent A: methyl tert-butyl ether+0.1 vol % diethylamine; eluent B: methanol; isocratic: 70% A+30% B; flow: 50 mL/min; temperature: 25° C.; UV: 254 nm;

Retention time: 14.0-17.0 min

Instrument: Thermo Fisher UltiMate 3000; Column: YMC Amylose SA 3μ, 100×4.6; eluent A: methyl tert-butyl ether+0.1 vol % diethylamine; eluent B: methanol; isocratic: 70% A+30% B; flow: 1.4 mL/min; temperature: 25° C.; UV: 254 nm

¹H-NMR (400 MHz, DMSO-d6) δ [ppm]: 0.752 (0.57), 0.834 (0.71), 0.851 (1.05), 0.864 (0.64), 1.006 (6.88), 1.024 (15.61), 1.041 (7.34), 1.102 (7.52), 1.120 (16.00), 1.138 (8.18), 1.161 (1.39), 1.232 (5.81), 1.256 (1.66), 1.278 (0.84), 1.295 (0.98), 1.332 (0.80), 1.348 (0.52), 1.776 (10.42), 1.793 (10.48), 2.007 (1.28), 2.020 (2.03), 2.036 (2.67), 2.054 (1.12), 2.066 (0.55), 2.467 (2.89), 2.518 (5.06), 2.522 (3.10), 2.794 (1.55), 2.812 (4.19), 2.830 (4.10), 2.848 (1.44), 3.027 (1.05), 3.045 (2.99), 3.060 (3.33), 3.063 (3.28), 3.077 (2.96), 3.095 (1.12), 3.333 (4.44), 3.351 (5.61), 3.358 (5.54), 3.394 (4.13), 3.447 (2.39), 3.465 (2.58), 3.479 (2.12), 3.486 (1.82), 3.504 (1.66), 4.109 (2.58), 4.127 (4.38), 4.144 (2.53), 5.785 (5.68), 6.022 (0.66), 6.039 (2.37), 6.056 (2.39), 6.072 (0.66), 6.131 (10.12), 6.139 (1.48), 6.153 (2.32), 6.166 (1.14), 7.079 (4.17), 7.083 (4.03), 7.412 (2.21), 7.434 (4.17), 7.456 (3.03), 7.535 (2.23), 7.547 (2.37), 7.558 (1.78), 7.570 (1.60), 7.879 (4.38), 7.882 (4.19), 8.332 (0.96).

### Example 615

### (3R)-2′-(6-amino-5-{[1-(6-methylpyridin-2-yl)ethyl]oxy}pyridin-3-yl)-N-ethyl-5′,6′-dihydrospiro[pyrrolidine-3,4′-pyrrolo[1,2-b]pyrazole]-1-carboxamide (Diastereomer 1)

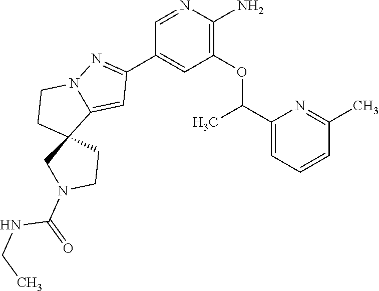 [SMILES: CCNC(=O)N1CC[C@@]2(CCn3nc(-c4cnc(N)c(OC(C)c5cccc(C)n5)c4)cc32)C1]

(3R)-1-(Ethylcarbamoyl)-5′,6′-dihydrospiro[pyrrolidine-3,4′-pyrrolo[1,2-b]pyrazol]-2′-yl trifluoromethanesulfonate (40.0 mg, 105 μmol), {6-amino-5-[1-(6-methylpyridin-2-yl)ethoxy]pyridin-3-yl}boronic acid (enantiomer 1) (Intermediate 432, 182 mg, 22% purity, 147 μmol) and potassium phosphate (630 μL, 0.50 M, 310 μmol) were combined in degassed 1,4-dioxane (1.7 mL) under argon. XPhos Pd G2 (4.12 mg, 5.24 μmol) was added and the mixture was stirred at 100° C. for 2 h. The reaction mixture was allowed to cool down, diluted with ethyl acetate and filtered. The filtrate was washed with water and brine, dried over sodium sulfate and concentrated under reduced pressure. The crude material was purified by flash chromatography (silica gel, dichloromethane/methanol (0-20%) gradient) followed by preparative TLC (gradient: dichloromethane/methanol 9:1) to give 19.8 mg (90% purity, 37% yield) of the title compound.

LC-MS (Method 1): Rₜ=0.88 min; MS (ESIpos): m/z=462 [M+H]⁺

¹H-NMR (400 MHz, CHLOROFORM-d): δ [ppm]=1.17 (t, 3H), 1.74 (d, 3H), 2.06 (ddd, 1H), 2.16-2.25 (m, 1H), 2.48-2.56 (m, 1H), 2.57 (s, 3H), 2.60 (br t, 1H), 3.27-3.35 (m, 2H), 3.48-3.56 (m, 3H), 3.57-3.65 (m, 1H), 4.16 (br t, 1H), 4.21 (t, 2H), 5.47 (d, 1H), 5.53-5.83 (m, 2H), 6.04 (s, 1H), 7.06 (d, 1H), 7.16 (d, 1H), 7.41 (d, 1H), 7.56 (t, 1H), 7.93 (d, 1H).

### Example 616

### (3R)-2′-(6-amino-5-{[1-(6-methylpyridin-2-yl)ethyl]oxy}pyridin-3-yl)-N-ethyl-5′,6′-dihydrospiro[pyrrolidine-3,4′-pyrrolo[1,2-b]pyrazole]-1-carboxamide (Diastereomer 2)

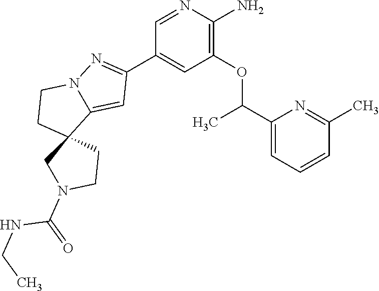 [SMILES: CCNC(=O)N1CC[C@@]2(CCn3nc(-c4cnc(N)c(OC(C)c5cccc(C)n5)c4)cc32)C1]

(3R)-1-(Ethylcarbamoyl)-5′,6′-dihydrospiro[pyrrolidine-3,4′-pyrrolo[1,2-b]pyrazol]-2′-yl trifluoromethanesulfonate (55.3 mg, 145 μmol), {6-amino-5-[1-(6-methylpyridin-2-yl)ethoxy]pyridin-3-yl}boronic acid (enantiomer 2) (Intermediate 433, 158 mg, 35% purity, 202 μmol) and potassium phosphate (870 μL, 0.50 M, 430 μmol) were combined in degassed 1,4-dioxane (2.4 mL) under argon. XPhos Pd G2 (5.69 mg, 7.23 μmol) was added and the mixture was stirred at 100° C. for 2 h. The reaction mixture was allowed to cool down, diluted with ethyl acetate and filtered. The filtrate was washed with water and brine. The organic phase was dried over sodium sulfate, filtered and concentrated under reduced pressure. The crude material was purified by flash column chromatography (silica gel, dichloromethane/methanol (0-20%) gradient) followed by preparative TLC (gradient: dichloromethane/methanol 9:1) to give 20.4 mg (95% purity, 29% yield) of the title compound.

LC-MS (Method 1): Rₜ=0.88 min; MS (ESIneg): m/z=460 [M−H]⁻

¹H-NMR (400 MHz, CHLOROFORM-d): δ [ppm]=1.17 (t, 3H), 1.73 (d, 3H), 2.06 (ddd, 1H), 2.17-2.25 (m, 1H), 2.48-2.66 (m, 5H), 3.27-3.35 (m, 2H), 3.47-3.55 (m, 3H), 3.57-3.65 (m, 1H), 4.16 (br t, 1H), 4.21 (t, H), 5.39-5.53 (m, 2H), 6.05 (s, 1H), 7.05 (d, 1H), 7.17 (d, 1H), 7.40 (d, 1H), 7.55 (t, 1H), 7.94 (d, 1H).

### Example 617

### (3R)-2′-(6-amino-5-{[1-(5-methylpyridin-2-yl)ethyl]oxy}pyridin-3-yl)-N-ethyl-5′,6′-dihydrospiro[pyrrolidine-3,4′-pyrrolo[1,2-b]pyrazole]-1-carboxamide (Diastereomer 1)

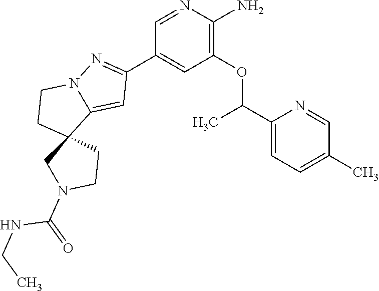 [SMILES: CCNC(=O)N1CC[C@@]2(CCn3nc(-c4cnc(N)c(OC(C)c5ccc(C)cn5)c4)cc32)C1]

The compound was prepared similarly to Example 615 using (3R)-1-(ethylcarbamoyl)-5′,6′-dihydrospiro[pyrrolidine-3,4′-pyrrolo[1,2-b]pyrazol]-2′-yl trifluoromethanesulfonate (40.4 mg, 106 μmol) and {6-amino-5-[1-(5-methylpyridin-2-yl)ethoxy]pyridin-3-yl}boronic acid (enantiomer 1) (Intermediate 434, 162 mg, 25% purity, 148 μmol) to give 12.9 mg (95% purity, 25% yield) of the title compound.

LC-MS (Method 1): Rₜ=0.88 min; MS (ESIpos): m/z=462 [M+H]⁺

¹H-NMR (400 MHz, DMSO-d6): δ [ppm]=1.02 (t, 3H), 1.59 (d, 3H), 2.01 (br d, 2H), 2.25 (s, 3H), 2.98-3.10 (m, 2H), 3.43-3.52 (m, 1H), 4.12 (t, 2H), 5.48 (d, 1H), 5.86 (s, 2H), 6.15 (s, 1H), 6.21 (s, 1H), 7.20 (d, 1H), 7.38 (d, 1H), 7.59 (dd, 1H), 7.85 (d, 1H), 8.34-8.39 (m, 1H).

### Example 618

(3R)-2′-(6-amino-5-{[1-(5-methylpyridin-2-yl)ethyl]oxy}pyridin-3-yl)-N-ethyl-5′,6′-dihydrospiro[pyrrolidine-3,4′-pyrrolo[1,2-b]pyrazole]-1-carboxamide (Diastereomer 2)

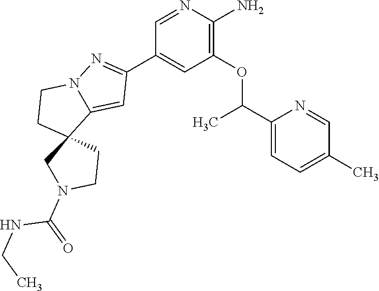 [SMILES: CCNC(=O)N1CC[C@@]2(CCn3nc(-c4cnc(N)c(OC(C)c5ccc(C)cn5)c4)cc32)C1]

The compound was prepared similarly to Example 615 using (3R)-1-(ethylcarbamoyl)-5′,6′-dihydrospiro[pyrrolidine-3,4′-pyrrolo[1,2-b]pyrazol]-2′-yl trifluoromethanesulfonate (44.7 mg, 117 μmol) and {6-amino-5-[1-(5-methylpyridin-2-yl)ethoxy]pyridin-3-yl}boronic acid (enantiomer 2) (Intermediate 435, 179 mg, 25% purity, 163 μmol) to give 13.6 mg (95% purity, 24% yield) of the title compound.

LC-MS (Method 1): Rₜ=0.88 min; MS (ESIpos): m/z=462 [M+H]⁺

¹H-NMR (400 MHz, CHLOROFORM-d): δ [ppm]=1.17 (t, 3H), 1.73 (d, 3H), 2.07 (ddd, 1H), 2.18-2.26 (m, 1H), 2.32 (s, 3H), 2.49-2.67 (m, 2H), 3.26-3.36 (m, 2H), 3.47-3.56 (m, 3H), 3.61 (td, 1H), 4.13-4.27 (m, 3H), 5.51 (q, 1H), 5.58-5.93 (m, 2H), 6.05 (s, 1H), 7.24 (s, 1H), 7.39-7.44 (m, 1H), 7.48 (dd, 1H), 7.88 (d, 1H), 8.41 (d, 1H).

### Example 619

### (3R)-2′-(6-amino-5-{[1-(4-methylpyridin-2-yl)ethyl]oxy}pyridin-3-yl)-N-ethyl-5′,6′-dihydrospiro[pyrrolidine-3,4′-pyrrolo[1,2-b]pyrazole]-1-carboxamide (Diastereomer 1)

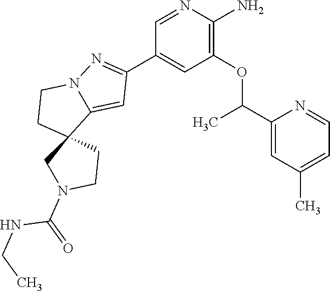 [SMILES: CCNC(=O)N1CC[C@@]2(CCn3nc(-c4cnc(N)c(OC(C)c5cc(C)ccn5)c4)cc32)C1]

(3R)-1-(Ethylcarbamoyl)-5′,6′-dihydrospiro[pyrrolidine-3,4′-pyrrolo[1,2-b]pyrazol]-2′-yl trifluoromethanesulfonate (28.5 mg, 74.6 μmol), {6-amino-5-[1-(4-methylpyridin-2-yl)ethoxy]pyridin-3-yl}boronic acid (enantiomer 1) (Intermediate 436, 143 mg, 20% purity, 105 μmol) and potassium phosphate (448 μL, 0.50 M, 224 μmol) were combined in degassed 1,4-dioxane (1.2 mL) under argon. XPhos Pd G2 (2.9 mg, 4 μmol) was added and the mixture was stirred at 100° C. for 2 h. The reaction mixture was allowed to cool down, diluted with ethyl acetate and filtered. The filtrated was washed with water and brine. The organic phase was dried over sodium sulfate, filtered and concentrated under reduced pressure. The crude material was purified by preparative TLC (gradient: dichloromethane/methanol 9:1) to give 7.2 mg (95% purity, 20% yield) of the title compound.

LC-MS (Method 1): Rₜ=0.87 min; MS (ESIpos): m/z=462 [M+H]⁺

¹H-NMR (400 MHz, CHLOROFORM-d): δ [ppm]=1.16 (t, 3H), 1.70 (d, 3H), 2.00-2.09 (m, 1H), 2.20 (br d, 1H), 2.31 (s, 3H), 2.45-2.55 (m, 1H), 2.56-2.66 (m, 1H), 3.25-3.37 (m, 2H), 3.46-3.55 (m, 3H), 3.56-3.65 (m, 1H), 4.13-4.26 (m, 3H), 4.87 (s, 2H), 5.47 (q, 1H), 6.03 (s, 1H), 7.00 (dd, 1H), 7.18 (s, 1H), 7.24 (d, 1H), 8.03 (d, 1H), 8.43 (d, 1H).

### Example 620

### (3R)-2′-(6-amino-5-{[1-(4-methylpyridin-2-yl)ethyl]oxy}pyridin-3-yl)-N-ethyl-5′,6′-dihydrospiro[pyrrolidine-3,4′-pyrrolo[1,2-b]pyrazole]-1-carboxamide (Diastereomer 2)

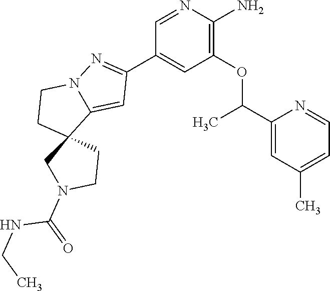 [SMILES: CCNC(=O)N1CC[C@@]2(CCn3nc(-c4cnc(N)c(OC(C)c5cc(C)ccn5)c4)cc32)C1]

(3R)-1-(Ethylcarbamoyl)-5′,6′-dihydrospiro[pyrrolidine-3,4′-pyrrolo[1,2-b]pyrazol]-2′-yl trifluoromethanesulfonate (28.7 mg, 75.1 μmol) and {6-amino-5-[1-(4-methylpyridin-2-yl)ethoxy]pyridin-3-yl}boronic acid (enantiomer 2) (Intermediate 437, 144 mg, 20% purity, 105 μmol) and potassium phosphate (450 μL, 0.50 M, 225 μmol) were combined in degassed 1,4-dioxane (1.2 mL) under argon. XPhos Pd G2 (2.9 mg, 4 μmol) was added and the mixture was stirred at 100° C. for 2 h. The reaction mixture was allowed to cool down, diluted with ethyl acetate and filtered. The filtrate was washed with water and brine. The organic phase was dried over sodium sulfate and concentrated under reduced pressure. The crude material was purified by flash column chromatography (silica gel, dichloromethane/methanol (0-15%) gradient) followed by preparative TLC (1 plate, 20×20 cm, gradient: dichloromethane/methanol 9:1) to give 6.7 mg (95% purity, 18% yield) of the title compound.

LC-MS (Method 1): Rₜ=0.87 min; MS (ESIpos): m/z=462 [M+H]⁺

¹H-NMR (400 MHz, CHLOROFORM-d): δ [ppm]=1.16 (t, 3H), 1.70 (d, 3H), 2.00-2.10 (m, 1H), 2.14-2.29 (m, 1H), 2.31 (s, 3H), 2.46-2.54 (m, 1H), 2.56-2.63 (m, 1H), 3.30 (qd, 2H), 3.45-3.56 (m, 3H), 3.56-3.66 (m, 1H), 4.12-4.26 (m, 3H), 4.87 (s, 2H), 5.47 (d, 1H), 6.04 (s, 1H), 7.00 (dd, 1H), 7.18 (s, 1H), 7.24-7.26 (m, 1H), 8.02 (d, 1H), 8.43 (d, 1H).

### Example 621

### (3R)-2′-(6-amino-5-{[1-(3-methylpyridin-2-yl)ethyl]oxy}pyridin-3-yl)-N-ethyl-5′,6′-dihydrospiro[pyrrolidine-3,4′-pyrrolo[1,2-b]pyrazole]-1-carboxamide (Diastereomer 1)

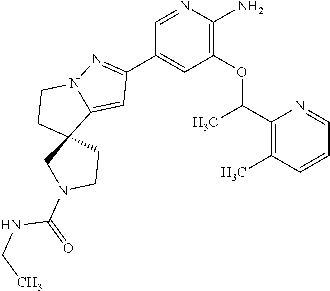 [SMILES: CCNC(=O)N1CC[C@@]2(CCn3nc(-c4cnc(N)c(OC(C)c5ncccc5C)c4)cc32)C1]

The compound was prepared similarly to Example 619 using (3R)-1-(ethylcarbamoyl)-5′,6′-dihydrospiro[pyrrolidine-3,4′-pyrrolo[1,2-b]pyrazol]-2′-yl trifluoromethanesulfonate (13.1 mg, 34.4 μmol) and {6-amino-5-[1-(3-methylpyridin-2-yl)ethoxy]pyridin-3-yl}boronic acid (enantiomer 1) (Intermediate 438, 87.6 mg, 15% purity, 48.1 μmol) to give 2.7 mg (95% purity, 16% yield) of the title compound.

LC-MS (Method 1): Rₜ=0.85 min; MS (ESIpos): m/z=462 [M+H]⁺

¹H-NMR (400 MHz, CHLOROFORM-d): δ [ppm]=1.17 (t, 3H), 1.76 (d, 3H), 2.01-2.12 (m, 1H), 2.17-2.26 (m, 1H), 2.39 (s, 3H), 2.48-2.57 (m, 1H), 2.58-2.68 (m, 1H), 3.26-3.36 (m, 2H), 3.50-3.57 (m, 3H), 3.58-3.67 (m, 1H), 4.13-4.20 (m, 1H), 4.23 (t, 2H), 4.87 (s, 2H), 5.67 (d, 1H), 6.05 (s, 1H), 7.14 (dd, 1H), 7.32 (d, 1H), 7.44 (br d, 1H), 8.01 (d, 1H), 8.46 (dd, 1H).

### Example 622

### (3R)-2′-(6-amino-5-{[1-(3-methylpyridin-2-yl)ethyl]oxy}pyridin-3-yl)-N-ethyl-5′,6′-dihydrospiro[pyrrolidine-3,4′-pyrrolo[1,2-b]pyrazole]-1-carboxamide (Diastereomer 2)

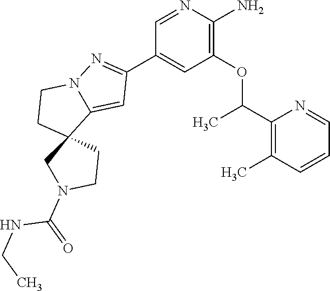 [SMILES: CCNC(=O)N1CC[C@@]2(CCn3nc(-c4cnc(N)c(OC(C)c5ncccc5C)c4)cc32)C1]

The compound was prepared similarly to Example 619 using (3R)-1-(ethylcarbamoyl)-5′,6′-dihydrospiro[pyrrolidine-3,4′-pyrrolo[1,2-b]pyrazol]-2′-yl trifluoromethanesulfonate (16.8 mg, 43.8 μmol) and {6-amino-5-[1-(3-methylpyridin-2-yl)ethoxy]pyridin-3-yl}boronic acid (enantiomer 2) (Intermediate 439, 83.8 mg, 20% purity, 61.4 μmol) to give 7.7 mg (95% purity, 36% yield) of the title compound.

LC-MS (Method 1): Rₜ=0.85 min; MS (ESIpos): m/z=462 [M+H]⁺

¹H-NMR (400 MHz, CHLOROFORM-d): δ [ppm]=1.17 (t, 3H), 1.76 (d, 3H), 2.07 (br d, 1H), 2.17-2.27 (m, 1H), 2.39 (s, 3H), 2.50-2.57 (m, 1H), 2.59-2.66 (m, 1H), 3.26-3.37 (m, 2H), 3.46-3.57 (m, 3H), 3.58-3.67 (m, 1H), 4.11-4.19 (m, 1H), 4.23 (t, 2H), 4.84 (s, 2H), 5.67 (d, 1H), 6.06 (s, 1H), 7.14 (dd, 1H), 7.34 (d, 1H), 7.44 (s, 1H), 8.01 (d, 1H), 8.47 (d, 1H).

### Example 623

### (3R)-2′-(6-amino-5-{[1-(1,4-dimethyl-1H-pyrazol-5-yl)ethyl]oxy}pyridin-3-yl)-N-ethyl-5′,6′-dihydrospiro[pyrrolidine-3,4′-pyrrolo[1,2-b]pyrazole]-1-carboxamide (Diastereomer 1)

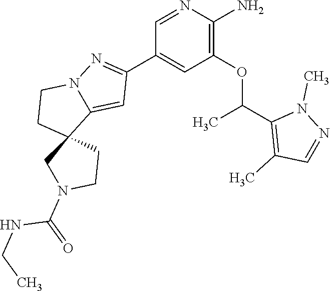 [SMILES: CCNC(=O)N1CC[C@@]2(CCn3nc(-c4cnc(N)c(OC(C)c5c(C)cnn5C)c4)cc32)C1]

(3R)-1-(Ethylcarbamoyl)-5′,6′-dihydrospiro[pyrrolidine-3,4′-pyrrolo[1,2-b]pyrazol]-2′-yl trifluoromethanesulfonate (51.5 mg, 135 μmol), 3-[1-(1,4-dimethyl-1H-pyrazol-5-yl)ethoxy]-5-(4,4,5,5-tetramethyl-1,3,2-dioxaborolan-2-yl)pyridin-2-amine (enantiomer 1) (Intermediate 440, 135 mg, 50% purity, 188 μmol) and potassium phosphate (807 μL, 0.50 M, 404 μmol) were combined in degassed 1,4-dioxane (2.2 mL) under argon. XPhos Pd G2 (5.2 mg, 7 μmol) was added and the mixture was stirred at 100° C. for 2 h. The reaction mixture was allowed to cool down, diluted with ethyl acetate and filtered. The filtrate was washed with water and brine. The organic phase was dried over sodium sulfate, filtered and concentrated under reduced pressure. The crude material was purified by flash column chromatography (silica gel, dichloromethane/methanol (0-20%) gradient) and TLC (gradient: dichloromethane/methanol 85:15) to give 20.3 mg (97% purity, 31% yield) of the title compound.

LC-MS (Method 1): Rₜ=0.84 min; MS (ESIpos): m/z=465 [M+H]⁺

¹H-NMR (400 MHz, DMSO-d6): δ [ppm]=1.02 (t, 3H), 1.66 (d, 3H), 1.97-2.07 (m, 2H), 2.08 (s, 3H), 3.00-3.11 (m, 2H), 3.43-3.52 (m, 1H), 3.85 (s, 3H), 4.14 (s, 2H), 5.71 (d, 1H), 5.85 (s, 2H), 6.12-6.18 (m, 1H), 6.19 (s, 1H), 7.11 (s, 1H), 7.14 (d, 1H), 7.87 (d, 1H).

[α]²⁰_{D}: −11.5° (c=1.00, MeOH)

### Example 624

### (3R)-2′-(6-amino-5-{[1-(1,4-dimethyl-1H-pyrazol-5-yl)ethyl]oxy}pyridin-3-yl)-N-ethyl-5′,6′-dihydrospiro[pyrrolidine-3,4′-pyrrolo[1,2-b]pyrazole]-1-carboxamide (Diastereomer 2)

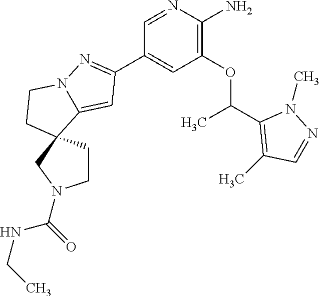 [SMILES: CCNC(=O)N1CC[C@@]2(CCn3nc(-c4cnc(N)c(OC(C)c5c(C)cnn5C)c4)cc32)C1]

The compound was prepared similarly to Example 623 using (3R)-1-(ethylcarbamoyl)-5′,6′-dihydrospiro[pyrrolidine-3,4′-pyrrolo[1,2-b]pyrazol]-2′-yl trifluoromethanesulfonate (41.9 mg, 110 μmol) and 3-[1-(1,4-dimethyl-1H-pyrazol-5-yl)ethoxy]-5-(4,4,5,5-tetramethyl-1,3,2-dioxaborolan-2-yl)pyridin-2-amine (enantiomer 2) (110 mg, 50% purity, 154 μmol) to give 22.2 mg (98% purity, 43% yield) of the title compound.

LC-MS (Method 1): Rₜ=0.84 min; MS (ESIpos): m/z=465 [M+H]⁺

¹H-NMR (400 MHz, DMSO-d6): δ [ppm]=1.02 (t, 3H), 1.66 (d, 3H), 1.98-2.06 (m, 2H), 2.08 (s, 3H), 2.99-3.10 (m, 2H), 3.37-3.42 (m, 3H), 3.43-3.53 (m, 1H), 3.86 (s, 3H), 4.14 (t, 2H), 5.69-5.75 (m, 1H), 5.85 (s, 2H), 6.12-6.18 (m, 1H), 6.19 (s, 1H), 7.12 (s, 1H), 7.14 (d, 1H), 7.87 (d, 1H).

[α]²⁰_{D}: +107.0° (c=1.00, MeOH)

### Example 625

### (3R)-2′-(6-amino-5-{[−1-(1,4-dimethyl-1H-pyrazol-5-yl)ethyl]oxy}pyridin-3-yl)-N-(propan-2-yl)-5′,6′-dihydrospiro[pyrrolidine-3,4′-pyrrolo[1,2-b]pyrazole]-1-carboxamide (Diastereomer 1)

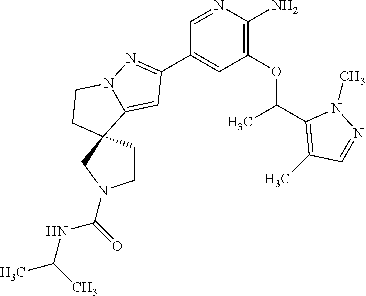 [SMILES: Cc1cnn(C)c1C(C)Oc1cc(-c2cc3n(n2)CC[C@@]32CCN(C(=O)NC(C)C)C2)cnc1N]

The compound was prepared similarly to Example 623 using (3R)-1-[(propan-2-yl)carbamoyl]-5′,6′-dihydrospiro[pyrrolidine-3,4′-pyrrolo[1,2-b]pyrazol]-2′-yl trifluoromethanesulfonate (34.3 mg, 86.4 μmol) and {6-amino-5-[1-(1,4-dimethyl-1H-pyrazol-5-yl)ethoxy]pyridin-3-yl}boronic acid (enantiomer 1) (Intermediate 441, 167 mg, 20% purity, 121 μmol) to give 12.9 mg (85% purity, 26% yield) of the title compound.

LC-MS (Method 1): Rₜ=0.88 min; MS (ESIpos): m/z=480 [M+H]⁺

¹H-NMR (400 MHz, DMSO-d6): δ [ppm]=1.06 (dd, 6H), 1.66 (d, 3H), 2.03 (dt, 2H), 2.08 (s, 3H), 3.36-3.42 (m, 3H), 3.44-3.53 (m, 1H), 3.70-3.81 (m, 1H), 3.86 (s, 3H), 4.10-4.16 (m, 2H), 5.71 (d, 1H), 5.80-5.90 (m, 3H), 6.19-6.20 (m, 1H), 7.11-7.13 (m, 1H), 7.13-7.16 (m, 1H), 7.87 (d, 1H).

### Example 626

### (3R)-2′-(6-amino-5-{[1-(1,4-dimethyl-1H-pyrazol-5-yl)ethyl]oxy}pyridin-3-yl)-N-(propan-2-yl)-5′,6′-dihydrospiro[pyrrolidine-3,4′-pyrrolo[1,2-b]pyrazole]-1-carboxamide (Diastereomer 2)

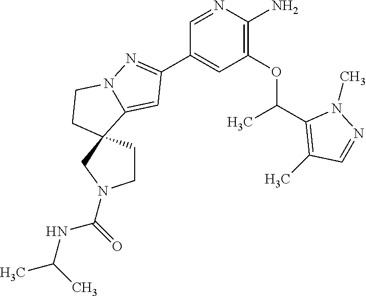 [SMILES: Cc1cnn(C)c1C(C)Oc1cc(-c2cc3n(n2)CC[C@@]32CCN(C(=O)NC(C)C)C2)cnc1N]

(3R)-1-[(Propan-2-yl)carbamoyl]-5′,6′-dihydrospiro[pyrrolidine-3,4′-pyrrolo[1,2-b]pyrazol]-2′-yl trifluoromethanesulfonate (29.7 mg, 75.0 μmol), {6-amino-5-[1-(1,4-dimethyl-1H-pyrazol-5-yl)ethoxy]pyridin-3-yl}boronic acid (enantiomer 2) (Intermediate 443, 116 mg, 25% purity, 105 μmol) and potassium phosphate (450 μL, 0.50 M, 225 μmol) were combined in degassed 1,4-dioxane (1.2 mL) under argon. XPhos Pd G2 (2.9 mg, 4 μmol) was added and the mixture was stirred at 100° C. for 2 h. The reaction mixture was allowed to cool down, diluted with ethyl acetate and filtered. The filtrate was washed with water and brine. The organic phase was dried over sodium sulfate, filtered and concentrated under reduced pressure. The crude material was purified by flash column chromatography (silica gel, dichloromethane/methanol (0-20%) gradient) and preparative TLC (gradient: dichloromethane/methanol 85:15) to give 20.2 mg (90% purity, 50% yield) of the title compound.

LC-MS (Method 1): Rₜ=0.89 min; MS (ESIpos): m/z=480 [M+H]⁺

¹H-NMR (400 MHz, DMSO-d6): δ [ppm]=1.06 (dd, 6H), 1.66 (d, 3H), 2.03 (dt, 2H), 2.08 (s, 3H), 3.36-3.42 (m, 3H), 3.44-3.53 (m, 1H), 3.70-3.81 (m, 1H), 3.86 (s, 3H), 4.10-4.16 (m, 2H), 5.71 (d, 1H), 5.80-5.90 (m, 3H), 6.19-6.20 (m, 1H), 7.11-7.13 (m, 1H), 7.13-7.16 (m, 1H), 7.87 (d, 1H).

### Example 627

### (3R)-2′-{6-amino-5-[(1S)-1-(2,6-difluorophenyl)ethoxy]pyridin-3-yl}-N-ethyl-5′,6′-dihydrospiro[pyrrolidine-3,4′-pyrrolo[1,2-b]pyrazole]-1-carboxamide

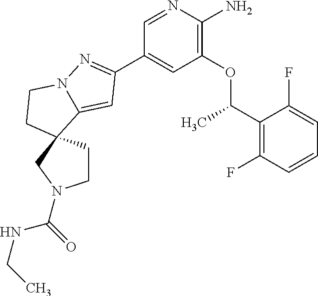 [SMILES: CCNC(=O)N1CC[C@@]2(CCn3nc(-c4cnc(N)c(O[C@@H](C)c5c(F)cccc5F)c4)cc32)C1]

(3R)-1-(Ethylcarbamoyl)-5′,6′-dihydrospiro[pyrrolidine-3,4′-pyrrolo[1,2-b]pyrazol]-2′-yl trifluoromethanesulfonate (50.0 mg, 131 μmol), {6-amino-5-[(1S)-1-(2,6-difluorophenyl)ethoxy]pyridin-3-yl}boronic acid (Intermediate 444, 179 mg, 30% purity, 183 μmol) and potassium phosphate (780 μL, 0.50 M, 390 μmol) were combined in degassed 1,4-dioxane (2.2 mL) under argon. XPhos Pd G2 (5.14 mg, 6.54 μmol) was added and the mixture was stirred at 100° C. for 2 h. The reaction mixture was allowed to cool down, diluted with ethyl acetate and filtered. The filtrate was washed with water and brine. The organic phase was dried over sodium sulfate, filtered and concentrated under reduced pressure. The crude material was purified by flash column chromatography (silica gel, dichloromethane/methanol (0-20%) gradient) followed by preparative TLC (gradient: dichloromethane/methanol 85:15) to give 38.6 mg (98% purity, 60% yield) of the title compound.

LC-MS (Method 1): Rₜ=1.01 min; MS (ESIneg): m/z=481 [M−H]⁻

¹H-NMR (400 MHz, DMSO-d6) δ [ppm]: 1.005 (6.76), 1.022 (15.75), 1.040 (6.99), 1.064 (2.82), 1.230 (0.46), 1.739 (8.10), 1.755 (7.96), 2.014 (1.17), 2.028 (1.96), 2.044 (2.52), 2.061 (0.97), 2.074 (0.48), 2.332 (1.07), 2.336 (0.47), 2.518 (6.40), 2.523 (3.70), 2.678 (0.47), 3.026 (0.83), 3.043 (2.65), 3.057 (2.93), 3.061 (2.83), 3.075 (2.58), 3.093 (0.75), 3.309 (1.22), 3.316 (1.55), 3.384 (13.46), 3.401 (1.75), 3.452 (0.82), 3.471 (1.25), 3.478 (0.83), 3.485 (0.97), 3.492 (0.76), 3.510 (0.44), 4.127 (2.42), 4.145 (4.06), 4.162 (2.32), 5.660 (5.50), 5.754 (16.00), 5.790 (0.57), 5.806 (1.90), 5.823 (1.90), 5.839 (0.55), 6.149 (1.08), 6.162 (2.12), 6.176 (1.08), 6.195 (10.62), 7.077 (2.57), 7.098 (5.40), 7.119 (3.05), 7.299 (3.98), 7.303 (3.98), 7.355 (0.51), 7.371 (1.14), 7.376 (0.94), 7.392 (1.92), 7.409 (0.95), 7.413 (1.04), 7.430 (0.43), 7.884 (5.51), 7.888 (5.42).

### Example 628

(3R)-2′-{6-amino-5-[(1R)-1-(5-fluoropyridin-2-yl)ethoxy]pyridin-3-yl}-N-ethyl-5′,6′-dihydrospiro[pyrrolidine-3,4′-pyrrolo[1,2-b]pyrazole]-1-carboxamide

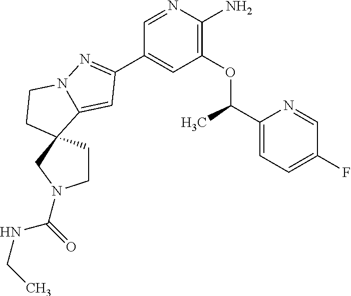 [SMILES: CCNC(=O)N1CC[C@@]2(CCn3nc(-c4cnc(N)c(O[C@H](C)c5ccc(F)cn5)c4)cc32)C1]

The compound was prepared similarly to Example 627 using (3R)-1-(ethylcarbamoyl)-5′,6′-dihydrospiro[pyrrolidine-3,4′-pyrrolo[1,2-b]pyrazol]-2′-yl trifluoromethanesulfonate (50.0 mg, 131 μmol) and {6-amino-5-[(1R)-1-(5-fluoropyridin-2-yl)ethoxy]pyridin-3-yl}boronic acid (Intermediate 445, 154 mg, 33% purity, 183 μmol) to give 27.0 mg (98% purity, 43% yield) of the title compound.

LC-MS (Method 1): Rₜ=0.88 min; MS (ESIpos): m/z=467 [M+H]⁺

¹H-NMR (400 MHz, DMSO-d6) δ [ppm]: 0.996 (7.01), 1.015 (15.96), 1.033 (7.37), 1.232 (0.43), 1.597 (10.31), 1.613 (10.22), 2.004 (1.27), 2.017 (2.02), 2.033 (2.68), 2.051 (1.08), 2.063 (0.52), 2.466 (2.50), 2.518 (5.65), 2.523 (3.45), 3.013 (0.92), 3.031 (2.92), 3.045 (3.21), 3.049 (3.17), 3.063 (2.83), 3.081 (0.84), 3.359 (3.50), 3.370 (9.80), 3.385 (2.46), 3.397 (0.83), 3.403 (0.94), 3.446 (0.77), 3.464 (1.32), 3.471 (0.85), 3.478 (1.00), 3.490 (0.80), 3.504 (0.49), 4.109 (2.68), 4.127 (4.75), 4.144 (2.57), 5.542 (0.67), 5.558 (2.42), 5.575 (2.40), 5.590 (0.66), 5.758 (16.00), 5.893 (6.63), 6.132 (1.18), 6.145 (2.38), 6.159 (1.16), 6.240 (10.86), 7.233 (4.45), 7.237 (4.29), 7.601 (1.72), 7.612 (1.80), 7.623 (2.54), 7.634 (2.40), 7.715 (1.53), 7.721 (1.70), 7.736 (2.58), 7.744 (2.76), 7.758 (1.11), 7.765 (1.14), 7.877 (6.16), 7.881 (6.03), 8.548 (4.34), 8.556 (4.26).

[α]²⁰_{D}: +121.1° (c=1.00, MeOH)

### Example 629

### (3R)-2′-{6-amino-5-[(1R)-1-(3-fluoropyridin-2-yl)ethoxy]pyridin-3-yl}-N-ethyl-5′,6′-dihydrospiro[pyrrolidine-3,4′-pyrrolo[1,2-b]pyrazole]-1-carboxamide

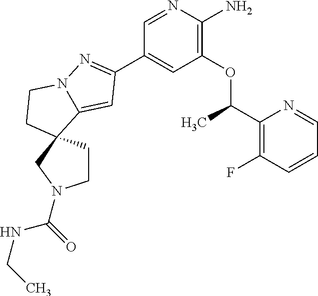 [SMILES: CCNC(=O)N1CC[C@@]2(CCn3nc(-c4cnc(N)c(O[C@H](C)c5ncccc5F)c4)cc32)C1]

The compound was prepared similarly to Example 627 using (3R)-1-(ethylcarbamoyl)-5′,6′-dihydrospiro[pyrrolidine-3,4′-pyrrolo[1,2-b]pyrazol]-2′-yl trifluoromethanesulfonate (50.0 mg, 131 μmol) and {6-amino-5-[(1R)-1-(3-fluoropyridin-2-yl)ethoxy]pyridin-3-yl}boronic acid (Intermediate 446, 145 mg, 35% purity, 183 μmol) to give 24.9 mg (98% purity, 40% yield) of the title compound.

LC-MS (Method 1): Rₜ=0.86 min; MS (ESIneg): m/z=464 [M−H]⁻

¹H-NMR (400 MHz, DMSO-d6) δ [ppm]: 1.002 (6.79), 1.020 (16.00), 1.038 (7.24), 1.232 (0.41), 1.494 (1.19), 1.684 (9.66), 1.700 (9.54), 2.013 (1.18), 2.026 (1.93), 2.042 (2.56), 2.060 (1.03), 2.072 (0.50), 2.332 (1.88), 2.336 (0.82), 2.518 (11.36), 2.523 (7.55), 2.673 (1.91), 2.678 (0.80), 3.020 (0.84), 3.038 (2.79), 3.052 (3.02), 3.056 (3.01), 3.070 (2.71), 3.088 (0.80), 3.366 (1.59), 3.382 (10.59), 3.391 (2.57), 3.409 (0.80), 3.449 (0.70), 3.468 (1.23), 3.475 (0.78), 3.482 (0.90), 3.493 (0.74), 3.508 (0.42), 4.124 (2.54), 4.142 (4.18), 4.159 (2.45), 5.680 (6.07), 5.743 (0.69), 5.759 (15.67), 5.775 (2.31), 5.791 (0.66), 6.141 (1.12), 6.155 (2.24), 6.169 (1.10), 6.212 (11.44), 7.308 (4.24), 7.312 (4.25), 7.435 (1.30), 7.445 (2.11), 7.456 (2.51), 7.467 (2.55), 7.478 (1.56), 7.714 (1.67), 7.717 (1.68), 7.735 (1.58), 7.739 (1.73), 7.740 (1.92), 7.744 (1.75), 7.761 (1.46), 7.765 (1.37), 7.884 (6.03), 7.888 (5.99), 8.436 (1.65), 8.440 (2.68), 8.444 (1.75), 8.447 (1.72), 8.451 (2.65), 8.455 (1.56).

### Example 630

### (3R)-2′-{6-amino-5-[(1R)-1-(2,6-difluorophenyl)ethoxy]pyridin-3-yl}-N-ethyl-5′,6′-dihydrospiro[pyrrolidine-3,4′-pyrrolo[1,2-b]pyrazole]-1-carboxamide

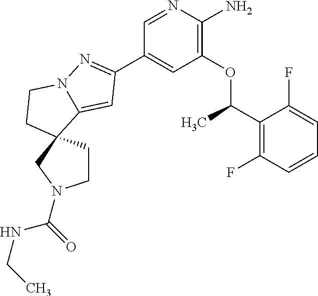 [SMILES: CCNC(=O)N1CC[C@@]2(CCn3nc(-c4cnc(N)c(O[C@H](C)c5c(F)cccc5F)c4)cc32)C1]

The compound was prepared similarly to Example 627 using (3R)-1-(ethylcarbamoyl)-5′,6′-dihydrospiro[pyrrolidine-3,4′-pyrrolo[1,2-b]pyrazol]-2′-yl trifluoromethanesulfonate (50.0 mg, 131 μmol) and {6-amino-5-[(1R)-1-(2,6-difluorophenyl)ethoxy]pyridin-3-yl}boronic acid (Intermediate 447, 146 mg, 37% purity, 183 μmol) to give 44.0 mg (98% purity, 68% yield) of the title compound.

LC-MS (Method 1): Rₜ=1.01 min; MS (ESIneg): m/z=481 [M−H]⁻

¹H-NMR (400 MHz, DMSO-d6) δ [ppm]: 1.003 (6.77), 1.021 (15.24), 1.038 (6.90), 1.066 (3.42), 1.233 (0.40), 1.739 (8.79), 1.755 (8.78), 2.016 (1.35), 2.030 (2.09), 2.043 (2.66), 2.061 (1.13), 2.074 (0.51), 2.332 (1.66), 2.522 (6.39), 3.022 (0.91), 3.039 (2.88), 3.054 (3.24), 3.057 (3.20), 3.072 (2.78), 3.089 (0.82), 3.295 (0.82), 3.304 (1.42), 3.363 (2.15), 3.366 (2.42), 3.384 (11.46), 3.410 (0.94), 3.448 (0.76), 3.466 (1.34), 3.473 (0.85), 3.480 (0.97), 3.488 (0.82), 3.506 (0.47), 4.128 (2.66), 4.145 (4.28), 4.162 (2.54), 5.663 (6.20), 5.758 (16.00), 5.787 (0.66), 5.805 (2.10), 5.821 (2.10), 5.837 (0.63), 6.145 (1.22), 6.159 (2.36), 6.172 (1.21), 6.195 (10.10), 7.083 (2.79), 7.105 (5.73), 7.126 (3.18), 7.298 (4.36), 7.302 (4.33), 7.362 (0.54), 7.378 (1.28), 7.382 (1.06), 7.399 (2.03), 7.415 (1.02), 7.420 (1.02), 7.436 (0.45), 7.887 (5.80), 7.891 (5.69).

### Example 631

### (3R)-2′-{6-amino-5-[1-(2-cyanophenyl)ethoxy]pyridin-3-yl}-N-ethyl-5′,6′-dihydro-1H-spiro[pyrrolidine-3,4′-pyrrolo[1,2-b]pyrazole]-1-carboxamide (Diastereomer 1 and Diastereomer 2)

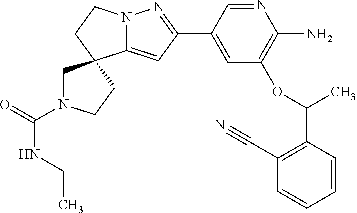 [SMILES: CCNC(=O)N1CC[C@@]2(CCn3nc(-c4cnc(N)c(OC(C)c5ccccc5C#N)c4)cc32)C1]

(3R)-1-(Ethylcarbamoyl)-5′,6′-dihydrospiro[pyrrolidine-3,4′-pyrrolo[1,2-b]pyrazol]-2′-yl trifluoromethanesulfonate (50.0 mg, 131 μmol), 2-(1-{[2-amino-5-(4,4,5,5-tetramethyl-1,3,2-dioxaborolan-2-yl)pyridin-3-yl]oxy}ethyl)benzonitrile (Intermediate 448, 167 mg, 40% purity, 183 μmol) and potassium phosphate (780 μL, 0.50 M, 390 μmol) were suspended in degassed 1,4-dioxane under argon. XPhos Pd G2 (5.14 mg, 6.54 μmol) was added and the mixture was stirred at 100° C. for 2 h. The reaction mixture was allowed to cool down, diluted with ethyl acetate and filtered. The filtrate was washed with water and brine. The organic phase was dried over sodium sulfate, filtered and concentrated under reduced pressure. The crude product was purified by flash column chromatography (silica gel, dichloromethane/methanol (0-10%) gradient) to give 24.7 mg (96% purity, 38% yield) of the title compound.

LC-MS (Method 1): Rₜ=0.94 min; MS (ESIpos): m/z=472 [M+H]⁺

¹H-NMR (400 MHz, CHLOROFORM-d) δ [ppm]: 0.887 (0.45), 1.156 (5.69), 1.167 (6.06), 1.175 (12.99), 1.185 (12.43), 1.192 (6.08), 1.203 (5.98), 1.261 (3.60), 1.292 (0.48), 1.762 (16.00), 1.779 (15.93), 2.040 (0.53), 2.055 (0.90), 2.070 (1.27), 2.085 (1.26), 2.101 (0.80), 2.204 (0.68), 2.220 (1.40), 2.237 (1.04), 2.252 (0.98), 2.269 (0.54), 2.503 (0.48), 2.518 (1.04), 2.521 (0.96), 2.536 (1.49), 2.551 (2.11), 2.568 (1.02), 2.593 (1.01), 2.610 (2.26), 2.625 (1.64), 2.640 (1.10), 2.643 (1.07), 2.658 (0.54), 3.283 (0.78), 3.296 (1.23), 3.301 (2.56), 3.308 (1.30), 3.314 (3.26), 3.319 (2.64), 3.326 (2.96), 3.330 (3.19), 3.332 (3.27), 3.337 (1.16), 3.344 (2.51), 3.349 (1.11), 3.362 (0.77), 3.482 (1.07), 3.490 (1.47), 3.507 (2.95), 3.514 (3.62), 3.532 (1.94), 3.540 (3.75), 3.551 (3.41), 3.565 (1.46), 3.575 (1.32), 3.603 (0.58), 3.609 (0.67), 3.616 (0.78), 3.622 (1.30), 3.628 (1.13), 3.635 (0.99), 3.641 (1.04), 3.646 (0.96), 3.660 (0.44), 4.179 (1.52), 4.191 (1.49), 4.205 (0.72), 4.218 (4.80), 4.236 (8.51), 4.253 (4.42), 4.882 (6.11), 5.751 (0.76), 5.767 (2.32), 5.782 (2.29), 5.799 (0.72), 6.098 (9.47), 6.104 (9.43), 7.233 (2.83), 7.238 (2.81), 7.254 (2.84), 7.259 (2.99), 7.377 (1.53), 7.383 (1.62), 7.394 (1.87), 7.397 (2.14), 7.399 (2.27), 7.402 (2.03), 7.413 (1.94), 7.418 (2.14), 7.529 (0.42), 7.560 (0.52), 7.565 (1.32), 7.580 (5.39), 7.582 (6.16), 7.585 (8.30), 7.598 (3.10), 7.601 (3.20), 7.604 (0.89), 7.618 (0.83), 7.622 (0.88), 7.691 (3.28), 7.693 (3.41), 7.695 (1.65), 7.712 (2.89), 8.038 (3.83), 8.042 (3.87), 8.053 (3.92), 8.058 (3.84).

### Example 632

### (3R)-2′-{6-amino-5-[1-(2-cyanophenyl)ethoxy]pyridin-3-yl}-N-ethyl-5′,6′-dihydro-1H-spiro[pyrrolidine-3,4′-pyrrolo[1,2-b]pyrazole]-1-carboxamide (Diastereomer 1)

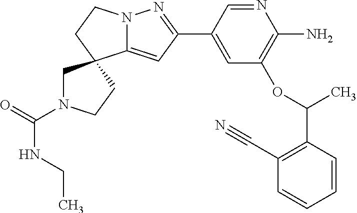 [SMILES: CCNC(=O)N1CC[C@@]2(CCn3nc(-c4cnc(N)c(OC(C)c5ccccc5C#N)c4)cc32)C1]

The title compound from Example 631 was separated into diastereomers by preparative chiral HPLC to give 6.43 mg (93% purity, 30% yield) of the title compound (Diastereomer 1, Rₜ=14.5-17.2 min)

Preparative Chiral HPLC Method:

Instrument: PrepCon Labomatic HPLC-1; Column: YMC Cellulose SC 5p, 250×30; eluent A: hexane+0.1 vol % diethylamine; eluent B: ethanol; isocratic: 40% A+60% B; flow: 40 mL/min; temperature: 25° C.; UV: 280 nm

Analytical Chiral HPLC Method:

Instrument: Thermo Fisher UltiMate 3000; Column: YMC Cellulose SC 3μ, 100×4.6; eluent A: hexane+0.1 vol % diethylamine; eluent B: ethanol; isocratic: 40% A+60% B; flow: 1.4 mL/min; temperature: 25° C.; UV: 280 nm

Analytical chiral HPLC: Rₜ=3.90 min.

[α]²⁰_{D}+40.9° (c=1.00, MeOH)

LC-MS (Method 1): Rₜ=0.94 min; MS (ESIpos): m/z=472 [M+H]⁺

¹H-NMR (400 MHz, DMSO-d6) δ [ppm]: 1.002 (4.30), 1.019 (10.04), 1.037 (4.62), 1.084 (0.51), 1.232 (0.91), 1.259 (0.86), 1.353 (3.52), 1.656 (5.92), 1.672 (5.99), 2.005 (0.84), 2.019 (1.13), 2.029 (1.58), 2.048 (0.68), 2.181 (0.47), 2.518 (16.00), 2.523 (10.97), 3.018 (0.55), 3.037 (1.75), 3.051 (1.94), 3.054 (1.89), 3.069 (1.71), 3.086 (0.52), 3.355 (1.21), 3.363 (0.92), 3.379 (5.31), 3.400 (0.56), 3.449 (0.43), 3.467 (0.75), 3.481 (0.58), 3.492 (0.47), 4.119 (1.56), 4.137 (2.54), 4.154 (1.49), 5.770 (1.40), 5.786 (1.39), 5.921 (3.76), 6.135 (0.75), 6.149 (1.44), 6.163 (0.69), 6.235 (6.83), 7.247 (2.61), 7.251 (2.55), 7.478 (0.90), 7.481 (0.89), 7.497 (1.83), 7.500 (1.77), 7.516 (1.16), 7.519 (1.10), 7.709 (0.76), 7.712 (0.78), 7.728 (1.55), 7.731 (1.60), 7.747 (1.15), 7.750 (1.12), 7.784 (1.98), 7.801 (1.11), 7.865 (1.74), 7.868 (1.77), 7.885 (1.65), 7.887 (1.64), 7.896 (3.58), 7.901 (3.42).

### Example 633

### (3R)-2′-{6-amino-5-[1-(2-cyanophenyl)ethoxy]pyridin-3-yl}-N-ethyl-5′,6′-dihydro-1H-spiro[pyrrolidine-3,4′-pyrrolo[1,2-b]pyrazole]-1-carboxamide (Diastereomer 2)

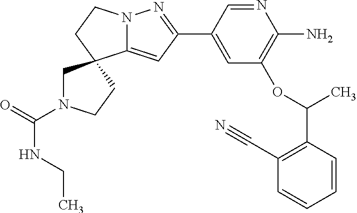 [SMILES: CCNC(=O)N1CC[C@@]2(CCn3nc(-c4cnc(N)c(OC(C)c5ccccc5C#N)c4)cc32)C1]

For the preparation of the diastereomeric mixture of the title compound see Example 631. The diastereomers were separated by preparative chiral HPLC (method see Example 632) to give 5.00 mg (94% purity) of the title compound (diastereomer 2, Rₜ=22.74-26.16 min).

Analytical chiral HPLC (method see Example 632): Rₜ=6.11 min.

[α]²⁰_{D}: +45.3° (c=1.00, MeOH)

LC-MS (Method 1): Rₜ=0.94 min; MS (ESIpos): m/z=472 [M+H]⁺

¹H-NMR (400 MHz, DMSO-d6) δ [ppm]: 0.795 (0.62), 0.814 (0.44), 0.862 (0.41), 0.997 (5.14), 1.006 (1.02), 1.015 (12.06), 1.033 (5.58), 1.084 (1.14), 1.233 (0.69), 1.259 (2.00), 1.353 (2.36), 1.655 (7.19), 1.671 (7.19), 2.009 (1.02), 2.016 (1.02), 2.023 (1.39), 2.034 (1.87), 2.051 (0.82), 2.331 (2.54), 2.336 (1.14), 2.518 (16.00), 2.523 (10.86), 2.673 (2.53), 2.678 (1.16), 3.013 (0.63), 3.031 (2.08), 3.046 (2.28), 3.049 (2.25), 3.063 (2.03), 3.081 (0.61), 3.362 (1.58), 3.372 (5.38), 3.387 (1.67), 3.402 (0.74), 3.447 (0.57), 3.465 (0.97), 3.479 (0.71), 3.490 (0.60), 4.118 (1.89), 4.136 (3.36), 4.153 (1.82), 5.756 (0.46), 5.772 (1.68), 5.788 (1.70), 5.804 (0.48), 5.920 (4.60), 6.129 (0.84), 6.143 (1.71), 6.157 (0.84), 6.241 (8.40), 7.253 (3.13), 7.256 (3.11), 7.481 (1.09), 7.484 (1.10), 7.500 (2.17), 7.503 (2.16), 7.519 (1.33), 7.522 (1.32), 7.710 (0.84), 7.714 (0.93), 7.730 (1.83), 7.733 (1.93), 7.749 (1.38), 7.752 (1.39), 7.787 (2.43), 7.804 (1.32), 7.866 (2.05), 7.868 (2.14), 7.885 (1.99), 7.888 (1.98), 7.898 (4.53), 7.902 (4.43), 10.851 (0.77).

### Example 634

### (3′R)-2-[6-amino-5-(trifluoromethyl)pyridin-3-yl]-N-[1-(1-ethyl-1H-pyrazol-3-yl)ethyl]-6,7-dihydro-1′H-spiro[pyrazolo[5,1-c][1,4]oxazine-4,3′-pyrrolidine]-1′-carboxamide (diastereomer 1 and diastereomer 2)

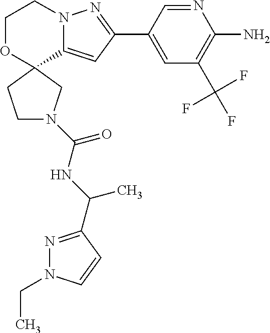 [SMILES: CCn1ccc(C(C)NC(=O)N2CC[C@]3(C2)OCCn2nc(-c4cnc(N)c(C(F)(F)F)c4)cc23)n1]

1-(1-Ethyl-1H-pyrazol-3-yl)ethan-1-amine (20.9 mg, 150 μmol) was dissolved in DMF (1.5 mL). CDI (22.4 mg, 138 μmol) and N,N-diisopropylethylamine (87 μL, 500 μmol) were added and the mixture was stirred for 1 h at rt. A solution of 5-[(3′R)-6,7-dihydrospiro[pyrazolo[5,1-c][1,4]oxazine-4,3′-pyrrolidin]-2-yl]-3-(trifluoromethyl)pyridin-2-amine-hydrogen chloride (47.1 mg, 125 μmol) in DMF was added dropwise. The reaction mixture was stirred for 1 h at 60° C. Molsieve 4 Å was added and the mixture was stirred for 1 h at rt. CDI (8.1 mg, 50 μmol) was added to the mixture and stirred for 1 h at 60° C. The reaction mixture was allowed to cool down, diluted with water and extracted 3 times with ethyl acetate. The combined organic layers were dried over a hydrophobic filter and concentrated to give a crude product. The residue was purified by preparative HPLC to give 49.5 mg (90% purity, 70% yield) of the title compound.

LC-MS (Method 1): Rₜ=0.97 min; MS (ESIpos): m/z=505 [M+H]⁺

¹H-NMR (400 MHz, CHLOROFORM-d) δ [ppm]: 0.006 (0.96), 0.960 (0.49), 1.122 (1.17), 1.132 (1.22), 1.139 (1.20), 1.150 (1.13), 1.183 (0.66), 1.201 (1.35), 1.219 (0.69), 1.260 (0.67), 1.324 (0.44), 1.341 (0.42), 1.440 (0.66), 1.450 (7.77), 1.455 (7.58), 1.468 (15.99), 1.473 (16.00), 1.486 (7.63), 1.492 (7.66), 1.499 (1.43), 1.516 (1.88), 1.524 (11.00), 1.540 (12.63), 2.269 (0.46), 2.294 (1.02), 2.301 (0.93), 2.327 (1.62), 2.350 (0.84), 2.400 (1.31), 2.416 (1.43), 2.433 (0.88), 2.450 (0.74), 3.149 (0.50), 3.168 (0.47), 3.591 (2.82), 3.594 (2.96), 3.603 (0.80), 3.610 (1.55), 3.619 (3.73), 3.623 (3.52), 3.634 (1.58), 3.642 (0.93), 3.652 (0.75), 3.658 (0.77), 3.677 (1.12), 3.696 (1.67), 3.717 (0.61), 3.953 (1.72), 3.982 (1.46), 4.101 (2.26), 4.107 (1.99), 4.119 (6.39), 4.124 (5.89), 4.138 (7.40), 4.143 (7.51), 4.148 (3.99), 4.156 (5.09), 4.161 (5.96), 4.169 (3.07), 4.226 (3.58), 4.237 (4.18), 5.009 (1.25), 5.030 (8.46), 5.044 (2.43), 5.054 (2.74), 5.070 (1.83), 5.075 (1.88), 5.087 (1.03), 5.093 (1.09), 6.114 (0.44), 6.120 (0.45), 6.140 (5.79), 6.145 (5.84), 6.283 (9.23), 6.288 (10.12), 7.305 (0.56), 7.310 (0.72), 7.316 (4.49), 7.319 (4.68), 7.321 (4.80), 7.324 (4.27), 8.105 (5.29), 8.110 (4.62), 8.588 (4.31), 8.593 (4.27).

### Example 635

### (3′R)-2-[6-amino-5-(trifluoromethyl)pyridin-3-yl]-N-[(1-methyl-1H-pyrazol-5-yl)methyl]-6,7-dihydrospiro[pyrazolo[5,1-c][1,4]oxazine-4,3′-pyrrolidine]-1′-carboxamide

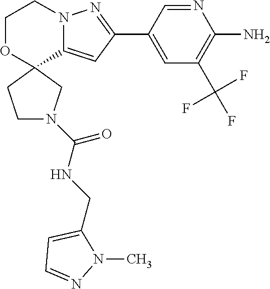 [SMILES: Cn1nccc1CNC(=O)N1CC[C@]2(C1)OCCn1nc(-c3cnc(N)c(C(F)(F)F)c3)cc12]

1-(1-Methyl-1H-pyrazol-5-yl)methanamine (16.7 mg, 150 μmol) was dissolved in DMF (1.5 mL). CDI (22.4 mg, 138 μmol) and N,N-diisopropylethylamine (87 μL, 500 μmol) were added and the mixture was stirred for 1 h at rt. A solution of 5-[(3′R)-6,7-dihydrospiro[pyrazolo[5,1-c][1,4]oxazine-4,3′-pyrrolidin]-2-yl]-3-(trifluoromethyl)pyridin-2-amine-hydrogen chloride (1/1) (47.1 mg, 125 μmol) in DMF was added dropwise. The reaction mixture was stirred over night at 60° C. Molsieve 4 Å was added and the mixture was stirred for 1 h at rt. CDI (8.1 mg, 50 μmol) was added to the mixture and stirred for 1 h at 60° C. The reaction mixture was allowed to cool down, diluted with water and extracted 3 times with ethyl acetate. The combined organic layers were dried over a hydrophobic filter and concentrated to give a crude product. The residue was purified by preparative HPLC and preparative TLC to give 16.8 mg (100% purity, 28% yield) of the title compound.

LC-MS (Method 1): Rₜ=0.81 min; MS (ESIpos): m/z=477 [M+H]⁺

¹H-NMR (400 MHz, CHLOROFORM-d) δ [ppm]: 1.260 (0.48), 1.615 (0.56), 2.339 (0.50), 2.429 (0.44), 2.445 (0.47), 3.569 (1.44), 3.585 (0.52), 3.598 (1.87), 3.611 (0.57), 3.627 (0.61), 3.641 (0.50), 3.902 (16.00), 3.955 (0.48), 3.984 (0.41), 4.133 (0.56), 4.141 (0.62), 4.146 (1.09), 4.152 (1.36), 4.162 (1.32), 4.230 (1.70), 4.242 (1.56), 4.252 (0.63), 4.257 (0.63), 4.400 (0.55), 4.502 (1.32), 4.514 (1.13), 4.521 (1.35), 4.535 (1.06), 5.046 (1.83), 5.308 (6.01), 6.185 (1.89), 6.190 (1.97), 6.285 (5.87), 7.414 (1.90), 7.419 (1.89), 8.101 (1.40), 8.106 (1.42), 8.584 (1.36), 8.588 (1.32).

### Example 636

### (3′R)-2-[6-amino-5-(trifluoromethyl)pyridin-3-yl]-N-[(1-methyl-1H-pyrazol-4-yl)methyl]-6,7-dihydrospiro[pyrazolo[5,1-c][1,4]oxazine-4,3′-pyrrolidine]-1′-carboxamide

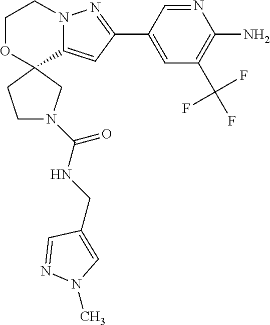 [SMILES: Cn1cc(CNC(=O)N2CC[C@]3(C2)OCCn2nc(-c4cnc(N)c(C(F)(F)F)c4)cc23)cn1]

1-(1-Methyl-1H-pyrazol-4-yl)methanamine (16.7 mg, 150 μmol) was dissolved in DMF (1.5 mL). CDI (22.4 mg, 138 μmol) and N,N-diisopropylethylamine (87 μL, 500 μmol) were added and the mixture was stirred for 1 h at rt. A solution of 5-[(3′R)-6,7-dihydrospiro[pyrazolo[5,1-c][1,4]oxazine-4,3′-pyrrolidin]-2-yl]-3-(trifluoromethyl)pyridin-2-amine-hydrogen chloride (47.1 mg, 125 μmol) in DMF was added dropwise. The reaction mixture was stirred for 1 h at 60° C. Molsieve 4 Å was added and the mixture was stirred for 1 h at rt. CDI (8.1 mg, 50 μmol) was added to the mixture and stirred for 1 h at 60° C. The reaction mixture was allowed to cool down, diluted with water and extracted 3 times with ethyl acetate. The combined organic layers were dried over a hydrophobic filter and concentrated to give a crude product. The residue was purified by preparative HPLC and preparative TLC to give 16.0 mg (100% purity, 27% yield) of the title compound.

LC-MS (Method 1): Rₜ=0.80 min; MS (ESIpos): m/z=477 [M+H]⁺

¹H-NMR (400 MHz, CHLOROFORM-d) δ [ppm]: 1.260 (0.56), 2.296 (0.42), 2.319 (0.58), 2.403 (0.48), 2.418 (0.52), 3.554 (1.77), 3.561 (0.63), 3.582 (2.05), 3.603 (0.84), 3.621 (0.57), 3.880 (16.00), 3.935 (0.61), 3.963 (0.53), 4.127 (0.63), 4.133 (0.70), 4.140 (1.20), 4.145 (1.59), 4.155 (1.63), 4.223 (1.89), 4.235 (1.54), 4.245 (0.70), 4.250 (0.71), 4.288 (1.38), 4.293 (1.44), 4.305 (2.11), 4.371 (0.51), 4.384 (0.70), 5.038 (2.26), 5.308 (5.12), 6.274 (5.86), 7.394 (2.95), 7.447 (2.94), 8.101 (1.55), 8.106 (1.58), 8.583 (1.49), 8.588 (1.45).

### Example 637

### (3′R)-2-[6-amino-5-(trifluoromethyl)pyridin-3-yl]-N-[1-(1-methyl-1H-pyrazol-5-yl)ethyl]-6,7-dihydro-1′H-spiro[pyrazolo[5,1-c][1,4]oxazine-4,3′-pyrrolidine]-1′-carboxamide (diastereomer 1 and diastereomer 2)

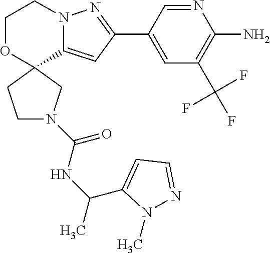 [SMILES: CC(NC(=O)N1CC[C@]2(C1)OCCn1nc(-c3cnc(N)c(C(F)(F)F)c3)cc12)c1ccnn1C]

1-(1-Methyl-1H-pyrazol-5-yl)ethan-1-amine (18.8 mg, 150 μmol) was dissolved in DMF (1.5 mL). CDI (22.4 mg, 138 μmol) and N,N-diisopropylethylamine (87 μL, 500 μmol) were added and the mixture was stirred for 1 h at rt. A solution of 5-[(3′R)-6,7-dihydrospiro[pyrazolo[5,1-c][1,4]oxazine-4,3′-pyrrolidin]-2-yl]-3-(trifluoromethyl)pyridin-2-amine-hydrogen chloride (47.1 mg, 125 μmol) in DMF was added dropwise. The reaction mixture was stirred for 1 h at 60° C. Molsieve 4 Å was added and the mixture was stirred for 1 h at rt. CDI (8.1 mg, 50 μmol) was added to the mixture and stirred for 1 h at 60° C. The reaction mixture was allowed to cool down, diluted with water and extracted 3 times with ethyl acetate. The combined organic layers were dried over a hydrophobic filter and concentrated to give a crude product. The residue was purified by preparative HPLC and preparative TLC again to give 16.3 mg (90% purity, 24% yield) of the title compound.

LC-MS (Method 1): Rₜ=0.88 min; MS (ESIpos): m/z=491 [M+H]⁺

¹H-NMR (400 MHz, CHLOROFORM-d) δ [ppm]: 1.261 (1.29), 1.558 (7.55), 1.575 (7.65), 2.296 (0.49), 2.328 (0.73), 2.416 (0.55), 2.421 (0.54), 2.432 (0.63), 3.539 (0.46), 3.552 (1.33), 3.558 (1.66), 3.580 (1.98), 3.587 (2.04), 3.603 (0.88), 3.622 (0.46), 3.903 (16.00), 3.906 (15.73), 3.920 (0.74), 3.951 (0.57), 4.135 (0.82), 4.147 (1.89), 4.160 (1.98), 4.228 (2.53), 4.240 (3.10), 4.250 (1.65), 4.263 (0.85), 5.038 (3.41), 5.228 (0.78), 5.245 (0.85), 5.248 (0.86), 5.266 (0.74), 5.309 (15.30), 6.190 (3.01), 6.195 (2.95), 6.278 (4.70), 6.289 (5.03), 6.345 (1.19), 7.412 (2.59), 7.416 (2.63), 8.098 (2.18), 8.104 (2.38), 8.581 (2.15), 8.586 (2.11).

### Example 638

### (3′R)-2-[6-amino-5-(trifluoromethyl)pyridin-3-yl]-N-[1-(1-methyl-1H-pyrazol-5-yl)ethyl]-6,7-dihydro-1′H-spiro[pyrazolo[5,1-c][1,4]oxazine-4,3′-pyrrolidine]-1′-carboxamide (diastereomer 1)

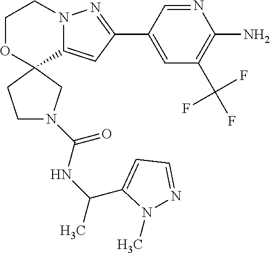 [SMILES: CC(NC(=O)N1CC[C@]2(C1)OCCn1nc(-c3cnc(N)c(C(F)(F)F)c3)cc12)c1ccnn1C]

The title compound from Example 637 was separated into diastereomers by preparative chiral HPLC to give 1.43 mg (97% purity) of the title compound (diastereomer 1, Rₜ=7.62-9.57 min)

Preparative Chiral HPLC Method: NPB

Instrument: PrepCon Labomatic HPLC-1; Column: YMC Cellulose SB 5μ, 250×30; eluent A: hexane+0.1 vol % diethylamine; eluent B: ethanol; isocratic: 80% A+20% B; flow: 90 mL/min; temperature: 25° C.; UV: 254 nm

Analytical Chiral HPLC Method: NPB

Instrument: Thermo Fisher UltiMate 3000; Column: YMC Cellulose SB 3μ, 100×4.6; eluent A: hexane+0.1 vol % diethylamine; eluent B: ethanol; isocratic: 80% A+20% B; flow: 1.4 mL/min; temperature: 25° C.; UV: 254 nm

Analytical chiral HPLC: Rₜ=3.78 min.

[α]²⁰_{D}: +94.5° (c=1.00, MeOH)

LC-MS (Method 1): Rₜ=0.89 min; MS (ESIpos): m/z=491 [M+H]⁺

¹H-NMR (400 MHz, CHLOROFORM-d) δ [ppm]: 1.225 (0.52), 1.264 (1.32), 1.277 (0.45), 1.406 (1.02), 1.440 (6.10), 1.558 (4.58), 1.575 (4.81), 2.280 (0.68), 2.325 (0.47), 2.328 (0.46), 2.416 (0.44), 2.433 (0.47), 3.539 (0.45), 3.552 (1.48), 3.565 (0.42), 3.580 (1.75), 3.622 (0.46), 3.906 (16.00), 3.924 (0.48), 4.133 (0.60), 4.137 (0.61), 4.147 (1.36), 4.160 (1.63), 4.226 (1.73), 4.236 (1.46), 4.241 (2.16), 4.249 (0.78), 4.254 (0.77), 4.263 (0.71), 5.043 (2.02), 5.228 (0.50), 5.245 (0.55), 5.249 (0.53), 5.266 (0.47), 6.191 (1.79), 6.195 (1.81), 6.278 (5.19), 7.411 (1.94), 7.416 (1.92), 8.099 (1.33), 8.104 (1.35), 8.581 (1.28), 8.585 (1.24).

### Example 639

### (3′R)-2-[6-amino-5-(trifluoromethyl)pyridin-3-yl]-N-[1-(1-methyl-1H-pyrazol-5-yl)ethyl]-6,7-dihydro-1′H-spiro[pyrazolo[5,1-c][1,4]oxazine-4,3′-pyrrolidine]-1′-carboxamide (Diastereomer 2)

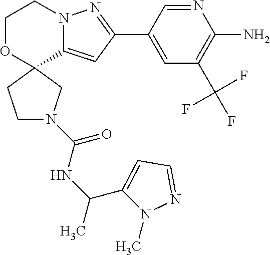 [SMILES: CC(NC(=O)N1CC[C@]2(C1)OCCn1nc(-c3cnc(N)c(C(F)(F)F)c3)cc12)c1ccnn1C]

For the preparation of the diastereomeric title compound see Example 637. The diastereomers were separated by preparative chiral HPLC (method see Example 638) to give 2.13 mg (98% purity) of the title compound (diastereomer 2, Rₜ=11.01-13.52 min). Analytical chiral HPLC (method see Example 638): Rₜ=5.75 min.

[α]²⁰_{D}: +73.4° (c=1.00, MeOH)

LC-MS (Method 1): Rₜ=0.88 min; MS (ESIpos): m/z=491 [M+H]⁺

¹H-NMR (400 MHz, CHLOROFORM-d) δ [ppm]: 1.224 (0.45), 1.261 (1.02), 1.406 (1.01), 1.439 (2.20), 1.558 (4.67), 1.575 (4.89), 2.297 (0.42), 2.330 (0.57), 2.435 (0.43), 3.558 (1.61), 3.578 (0.82), 3.587 (2.29), 3.603 (0.81), 3.903 (16.00), 3.919 (0.49), 4.135 (0.56), 4.149 (1.08), 4.153 (1.40), 4.164 (1.27), 4.229 (2.38), 4.241 (1.74), 4.244 (1.46), 4.251 (1.37), 5.048 (2.19), 5.226 (0.52), 5.243 (0.57), 5.248 (0.56), 5.265 (0.49), 6.190 (1.89), 6.194 (1.90), 6.289 (5.32), 7.413 (2.00), 7.418 (1.98), 8.100 (1.43), 8.105 (1.44), 8.580 (1.38), 8.584 (1.34).

### Example 640

### (3′R)-2-[6-amino-5-(trifluoromethyl)pyridin-3-yl]-N-[1-(1,3-dimethyl-1H-pyrazol-4-yl)ethyl]-6,7-dihydro-1′H-spiro[pyrazolo[5,1-c][1,4]oxazine-4,3′-pyrrolidine]-1′-carboxamide (Diastereomer 1 and Diastereomer 2)

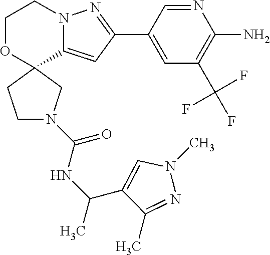 [SMILES: Cc1nn(C)cc1C(C)NC(=O)N1CC[C@]2(C1)OCCn1nc(-c3cnc(N)c(C(F)(F)F)c3)cc12]

1-(1,3-Dimethyl-1H-pyrazol-4-yl)ethan-1-amine (20.9 mg, 150 μmol) was dissolved in DMF (1.5 mL). CDI (22.4 mg, 138 μmol) and N,N-diisopropylethylamine (87 μL, 500 μmol) were added and the mixture was stirred for 1 h at rt. A solution of 5-[(3′R)-6,7-dihydrospiro[pyrazolo[5,1-c][1,4]oxazine-4,3′-pyrrolidin]-2-yl]-3-(trifluoromethyl)pyridin-2-amine-hydrogen chloride (47.1 mg, 125 μmol) in DMF was added dropwise. The reaction mixture was stirred for 1 h at 60° C. Molsieve 4 Å was added and the mixture was stirred for 1 h at rt. CDI (8.1 mg, 50 μmol) was added to the mixture and stirred for 1 h at 60° C. The reaction mixture was allowed to cool down, diluted with water and extracted 3 times with ethyl acetate. The combined organic layers were dried over a hydrophobic filter and concentrated to give a crude product. The residue was purified by preparative HPLC and preparative TLC to give 21.7 mg (90% purity, 31% yield) of the title compound.

LC-MS (Method 1): Rₜ=0.88 min; MS (ESIpos): m/z=505 [M+H]⁺

¹H-NMR (400 MHz, CHLOROFORM-d) δ [ppm]: 0.052 (0.46), 1.306 (1.22), 1.537 (7.05), 1.554 (7.05), 2.313 (12.88), 2.316 (12.17), 2.338 (0.59), 2.348 (0.47), 2.370 (0.82), 2.391 (0.49), 2.440 (0.69), 2.455 (0.76), 2.473 (0.50), 2.489 (0.41), 3.573 (0.60), 3.581 (1.45), 3.602 (1.82), 3.609 (2.40), 3.631 (2.21), 3.658 (0.54), 3.856 (14.40), 3.955 (0.81), 3.968 (0.67), 3.983 (0.58), 3.995 (0.56), 4.178 (1.13), 4.183 (1.07), 4.194 (2.82), 4.205 (3.22), 4.216 (1.21), 4.229 (1.10), 4.235 (1.13), 4.268 (2.67), 4.281 (2.51), 4.289 (1.13), 4.295 (0.94), 5.001 (1.10), 5.018 (1.54), 5.036 (1.05), 5.078 (3.97), 5.354 (16.00), 6.317 (5.33), 6.332 (5.66), 6.390 (1.01), 7.270 (3.87), 8.144 (2.53), 8.150 (2.72), 8.628 (2.48), 8.633 (2.42).

### Example 641

### (3′R)-2-[6-amino-5-(trifluoromethyl)pyridin-3-yl]-N-[1-(1,3-dimethyl-1H-pyrazol-4-yl)ethyl]-6,7-dihydro-1′H-spiro[pyrazolo[5,1-c][1,4]oxazine-4,3′-pyrrolidine]-1′-carboxamide (Diastereomer 1)

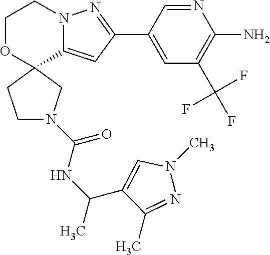 [SMILES: Cc1nn(C)cc1C(C)NC(=O)N1CC[C@]2(C1)OCCn1nc(-c3cnc(N)c(C(F)(F)F)c3)cc12]

The title compound from Example 640 was separated into diastereomers by preparative chiral HPLC to give 3.29 mg (99% purity) of the title compound (diastereomer 1, Rₜ=8.48-10.61 min)

Preparative Chiral HPLC Method: NPB

Instrument: PrepCon Labomatic HPLC-1; Column: YMC Cellulose SB 5μ, 250×30; eluent A: hexane+0.1 vol % diethylamine; eluent B: ethanol; isocratic: 80% A+20% B; flow: 90 mL/min; temperature: 25° C.; UV: 280 nm

Analytical Chiral HPLC Method: NPB

Instrument: Thermo Fisher UltiMate 3000; Column: YMC Cellulose SB 3μ, 100×4.6; eluent A: hexane+0.1 vol % diethylamine; eluent B: ethanol; isocratic: 80% A+20% B; flow: 1.4 mL/min; temperature: 25° C.; UV: 280 nm

Analytical chiral HPLC: Rₜ=4.11 min.

[α]²⁰_{D}: +55.4° (c=1.00, MeOH)

LC-MS (Method 1): Rₜ=0.89 min; MS (ESIpos): m/z=505 [M+H]⁺

¹H-NMR (400 MHz, CHLOROFORM-d) δ [ppm]: 1.268 (0.55), 1.305 (1.20), 1.449 (1.20), 1.483 (4.65), 1.536 (6.54), 1.553 (6.51), 2.311 (16.00), 2.323 (0.74), 2.330 (0.55), 2.341 (0.57), 2.356 (0.41), 2.363 (0.77), 2.389 (0.50), 2.437 (0.64), 2.454 (0.70), 2.470 (0.44), 2.669 (0.41), 3.579 (2.01), 3.588 (0.79), 3.597 (0.69), 3.608 (2.25), 3.638 (0.53), 3.656 (0.76), 3.853 (15.59), 3.965 (0.83), 3.994 (0.73), 4.176 (0.92), 4.180 (0.95), 4.190 (2.38), 4.202 (2.47), 4.216 (1.20), 4.234 (1.20), 4.265 (2.33), 4.274 (1.88), 4.279 (2.09), 4.287 (0.87), 4.292 (0.82), 4.998 (0.89), 5.015 (1.29), 5.033 (0.86), 5.067 (0.53), 5.082 (3.18), 6.316 (6.88), 7.270 (4.18), 8.143 (2.02), 8.147 (2.05), 8.625 (1.95), 8.629 (1.90).

### Example 642

### (3′R)-2-[6-amino-5-(trifluoromethyl)pyridin-3-yl]-N-[1-(1,3-dimethyl-1H-pyrazol-4-yl)ethyl]-6,7-dihydro-1′H-spiro[pyrazolo[5,1-c][1,4]oxazine-4,3′-pyrrolidine]-1′-carboxamide (Diastereomer 2)

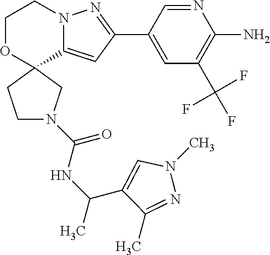 [SMILES: Cc1nn(C)cc1C(C)NC(=O)N1CC[C@]2(C1)OCCn1nc(-c3cnc(N)c(C(F)(F)F)c3)cc12]

For the preparation of the diastereomeric mixture of the title compound see Example 640. The diastereomers were separated by preparative chiral HPLC (method see Example 641) to give 2.62 mg (100% purity) of the title compound (enantiomer 2, Rₜ=10.61-13.77 min).

Analytical chiral HPLC (method see Example 641): Rₜ=5.05 min.

[α]²⁰_{D}: +58.7° (c=1.00, MeOH)

LC-MS (Method 1): Rₜ=0.88 min; MS (ESIpos): m/z=505 [M+H]⁺

¹H-NMR (400 MHz, CHLOROFORM-d) δ [ppm]: 0.936 (0.71), 1.197 (0.52), 1.221 (0.47), 1.271 (1.18), 1.309 (1.05), 1.452 (2.14), 1.487 (2.19), 1.538 (6.50), 1.555 (6.35), 1.613 (0.45), 1.632 (0.62), 2.318 (15.92), 2.340 (0.67), 2.349 (0.56), 2.364 (0.40), 2.373 (0.91), 2.397 (0.51), 2.454 (0.64), 3.603 (2.42), 3.611 (1.40), 3.622 (1.07), 3.632 (3.18), 3.860 (16.00), 3.956 (0.78), 3.985 (0.67), 4.179 (0.83), 4.185 (0.95), 4.192 (1.59), 4.196 (2.37), 4.207 (2.46), 4.232 (1.21), 4.270 (2.18), 4.280 (1.96), 4.283 (1.84), 4.291 (0.90), 4.298 (0.79), 5.003 (0.86), 5.021 (1.24), 5.038 (0.83), 5.088 (3.14), 6.333 (6.92), 7.270 (4.16), 8.148 (2.05), 8.152 (2.05), 8.630 (1.99), 8.633 (1.93).

### Example 643

### (3R)-2′-{6-amino-5-[(1R)-1-(3-cyanophenyl)ethoxy]pyridin-3-yl}-N-ethyl-5′,6′-dihydrospiro[pyrrolidine-3,4′-pyrrolo[1,2-b]pyrazole]-1-carboxamide

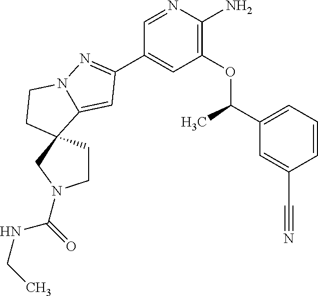 [SMILES: CCNC(=O)N1CC[C@@]2(CCn3nc(-c4cnc(N)c(O[C@H](C)c5cccc(C#N)c5)c4)cc32)C1]

(3R)-1-(Ethylcarbamoyl)-5′,6′-dihydrospiro[pyrrolidine-3,4′-pyrrolo[1,2-b]pyrazol]-2′-yl trifluoromethanesulfonate (40.0 mg, 105 μmol), {6-amino-5-[(1R)-1-(3-cyanophenyl)ethoxy]pyridin-3-yl}boronic acid (Intermediate 449, 109 mg, 38% purity, 146 μmol) and potassium phosphate (630 μL, 0.50 M, 310 μmol) were combined in degassed 1,4-dioxane (1.7 mL) under argon. XPhos Pd G2 (4.12 mg, 5.23 μmol) was added and the mixture was stirred for 2 h at 100° C. The reaction mixture was allowed to cool down, diluted with ethyl acetate and filtered. The filtrate was washed with water and brine, dried over sodium sulfate and concentrated under reduced pressure. The crude product was purified by preparative TLC (dichlormethan/methanol 9:1 gradient) to give 29.3 mg (98% purity, 58% yield) of the title compound.

LC-MS (Method 1): Rₜ=0.92 min; MS (ESIpos): m/z=473 [M+H]⁺

¹H-NMR (400 MHz, CHLOROFORM-d) δ [ppm]: 1.152 (7.47), 1.170 (16.00), 1.188 (7.65), 1.249 (1.06), 1.259 (0.41), 1.681 (12.44), 1.697 (13.15), 2.044 (0.54), 2.061 (0.79), 2.075 (1.27), 2.089 (1.14), 2.107 (0.78), 2.194 (0.80), 2.212 (1.44), 2.225 (0.82), 2.230 (1.02), 2.243 (0.96), 2.261 (0.57), 2.508 (0.54), 2.526 (1.18), 2.541 (1.47), 2.559 (1.87), 2.576 (1.11), 2.600 (1.11), 2.635 (1.40), 2.649 (1.30), 2.667 (0.60), 3.280 (1.08), 3.294 (1.46), 3.298 (3.38), 3.312 (3.67), 3.316 (3.48), 3.330 (3.30), 3.333 (1.42), 3.348 (1.02), 3.479 (1.47), 3.504 (3.74), 3.522 (1.23), 3.529 (1.27), 3.538 (4.45), 3.546 (2.03), 3.562 (2.33), 3.599 (0.77), 3.613 (1.02), 3.618 (1.17), 3.632 (0.96), 3.637 (0.82), 3.642 (0.65), 3.656 (0.44), 4.190 (0.97), 4.204 (1.80), 4.216 (4.31), 4.234 (6.25), 4.251 (3.22), 4.796 (5.90), 5.308 (10.54), 5.467 (0.68), 5.483 (2.20), 5.499 (2.18), 5.515 (0.65), 6.073 (11.41), 6.085 (0.42), 7.210 (4.26), 7.214 (4.29), 7.459 (1.58), 7.478 (4.07), 7.498 (2.82), 7.573 (1.82), 7.576 (3.20), 7.580 (2.16), 7.592 (1.42), 7.595 (2.23), 7.599 (1.43), 7.623 (1.63), 7.626 (2.55), 7.646 (2.01), 7.701 (4.27), 8.006 (5.31), 8.011 (5.44).

### Example 644

### (3R)-2′-{6-amino-5-[(1R)-1-(4-cyanophenyl)ethoxy]pyridin-3-yl}-N-ethyl-5′,6′-dihydrospiro[pyrrolidine-3,4′-pyrrolo[1,2-b]pyrazole]-1-carboxamide

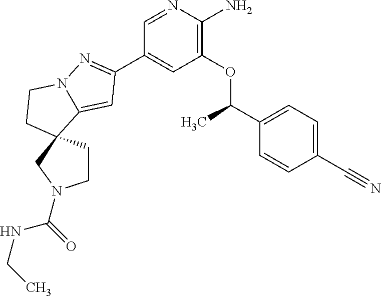 [SMILES: CCNC(=O)N1CC[C@@]2(CCn3nc(-c4cnc(N)c(O[C@H](C)c5ccc(C#N)cc5)c4)cc32)C1]

The compound was prepared similarly to Example 643 using (3R)-1-(ethylcarbamoyl)-5′,6′-dihydrospiro[pyrrolidine-3,4′-pyrrolo[1,2-b]pyrazol]-2′-yl trifluoromethanesulfonate (40.0 mg, 105 μmol) and {6-amino-5-[(1R)-1-(4-cyanophenyl)ethoxy]pyridin-3-yl}boronic acid (Intermediate 450, 92.1 mg, 45% purity, 146 μmol) to give 30.7 mg (98% purity, 61% yield) of the title compound.

LC-MS (Method 1): Rₜ=0.91 min; MS (ESIpos): m/z=473 [M+H]⁺

¹H-NMR (400 MHz, CHLOROFORM-d) δ [ppm]: 0.006 (0.47), 1.151 (6.99), 1.169 (16.00), 1.188 (7.05), 1.250 (0.91), 1.656 (2.29), 1.679 (11.12), 1.695 (10.34), 2.039 (0.48), 2.054 (0.67), 2.057 (0.66), 2.071 (1.15), 2.084 (0.99), 2.088 (0.89), 2.102 (0.70), 2.189 (0.72), 2.208 (1.31), 2.220 (0.68), 2.226 (0.88), 2.239 (0.87), 2.257 (0.50), 2.505 (0.48), 2.523 (0.92), 2.538 (1.30), 2.554 (1.32), 2.557 (1.50), 2.572 (0.99), 2.594 (0.98), 2.630 (1.21), 2.644 (1.02), 2.662 (0.51), 3.280 (1.03), 3.294 (1.34), 3.297 (3.08), 3.311 (3.44), 3.316 (3.28), 3.330 (3.11), 3.333 (1.19), 3.347 (0.97), 3.474 (1.21), 3.499 (3.04), 3.527 (4.33), 3.546 (1.72), 3.551 (1.75), 3.564 (0.70), 3.592 (0.68), 3.606 (0.87), 3.611 (0.99), 3.625 (0.82), 3.635 (0.51), 4.155 (0.82), 4.169 (1.44), 4.183 (0.78), 4.205 (2.65), 4.224 (4.73), 4.242 (2.23), 4.791 (5.14), 5.308 (13.37), 5.477 (0.59), 5.493 (1.94), 5.509 (1.91), 5.526 (0.57), 6.054 (10.60), 7.170 (3.91), 7.174 (3.90), 7.493 (4.89), 7.514 (6.33), 7.646 (1.38), 7.650 (7.20), 7.654 (2.26), 7.666 (2.04), 7.671 (5.50), 7.675 (0.92), 8.003 (5.46), 8.007 (5.34).

### Example 645

### (3R)-2′-{6-amino-5-[(1R)-1-(3,5-difluoropyridin-4-yl)ethoxy]pyridin-3-yl}-N-ethyl-5′,6′-dihydrospiro[pyrrolidine-3,4′-pyrrolo[1,2-b]pyrazole]-1-carboxamide

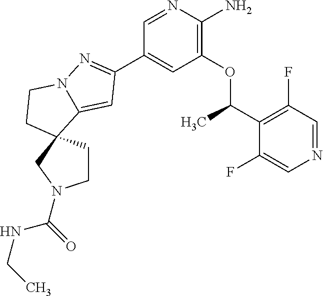 [SMILES: CCNC(=O)N1CC[C@@]2(CCn3nc(-c4cnc(N)c(O[C@H](C)c5c(F)cncc5F)c4)cc32)C1]

The compound was prepared similarly to Example 643 using (3R)-1-(ethylcarbamoyl)-5′,6′-dihydrospiro[pyrrolidine-3,4′-pyrrolo[1,2-b]pyrazol]-2′-yl trifluoromethanesulfonate (40.0 mg, 105 μmol) and {6-amino-5-[(1R)-1-(3,5-difluoropyridin-4-yl)ethoxy]pyridin-3-yl}boronic acid (Intermediate 451, 100 mg, 43% purity, 146 μmol) to give 31.7 mg (98% purity, 61% yield) of the title compound.

[α]²⁰_{D}: +198.9° (c=1.00, MeOH)

LC-MS (Method 1): Rₜ=0.86 min; MS (ESIpos): m/z=485 [M+H]⁺

¹H-NMR (400 MHz, CHLOROFORM-d) δ [ppm]: 0.007 (0.52), 1.158 (6.50), 1.176 (14.25), 1.194 (6.57), 1.250 (0.69), 1.814 (9.06), 1.830 (8.92), 2.060 (0.50), 2.074 (0.67), 2.078 (0.68), 2.092 (1.30), 2.104 (0.98), 2.123 (0.67), 2.220 (0.72), 2.239 (1.39), 2.251 (0.69), 2.256 (0.89), 2.269 (0.96), 2.288 (0.50), 2.526 (0.48), 2.543 (1.12), 2.558 (1.30), 2.576 (1.82), 2.593 (0.98), 2.634 (2.04), 2.651 (1.22), 2.667 (1.14), 2.684 (0.51), 3.288 (1.00), 3.302 (1.39), 3.305 (2.96), 3.319 (3.33), 3.324 (3.18), 3.338 (2.98), 3.342 (1.21), 3.356 (0.91), 3.504 (1.27), 3.516 (0.70), 3.529 (3.22), 3.539 (1.13), 3.558 (5.11), 3.576 (0.88), 3.582 (1.54), 3.616 (0.70), 3.629 (0.87), 3.635 (1.00), 3.649 (0.85), 3.659 (0.54), 4.160 (0.85), 4.173 (1.47), 4.186 (0.77), 4.245 (3.19), 4.262 (5.96), 4.280 (3.01), 4.785 (5.11), 5.309 (13.07), 5.874 (0.64), 5.890 (2.04), 5.906 (2.01), 5.923 (0.61), 6.122 (9.74), 7.401 (3.80), 7.405 (3.82), 8.040 (4.83), 8.044 (4.64), 8.346 (16.00).

### Example 646

### (3R)-2′-[6-amino-5-(1-phenylpropoxy)pyridin-3-yl]-N-ethyl-5′,6′-dihydro-1H-spiro[pyrrolidine-3,4′-pyrrolo[1,2-b]pyrazole]-1-carboxamide (Diastereomer 1 and Diastereomer 2)

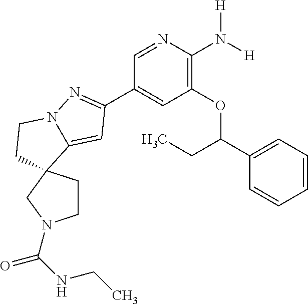 [SMILES: CCNC(=O)N1CC[C@@]2(CCn3nc(-c4cnc(N)c(OC(CC)c5ccccc5)c4)cc32)C1]

(3R)-1-(Ethylcarbamoyl)-5′,6′-dihydrospiro[pyrrolidine-3,4′-pyrrolo[1,2-b]pyrazol]-2′-yl trifluoromethanesulfonate (235 mg, 615 μmol), [6-amino-5-(1-phenylpropoxy)pyridin-3-yl]boronic acid (Intermediate 452, 551 mg, 33% purity, 677 μmol) and potassium phosphate (3.7 mL, 0.50 M, 1.8 mmol) were suspended in degassed 1,4-dioxane (6.9 mL) under nitrogen. XPhos Pd G2 (24.2 mg, 30.8 μmol) was added and the mixture was stirred over night at 100° C. The reaction mixture was cooled to rt, diluted with ethyl acetate, filtered over celite and washed with water and brine. The organic layer was dried over a hydrophobic filter and concentrated to give a residue. The crude product was purified by flash chromatography (silica gel, dichloromethane/methanol 9:1 gradient) and preparative TLC to give 9.20 mg (90% purity, 3% yield) of the title compound.

LC-MS (Method 1): Rₜ=1.05 min; MS (ESIpos): m/z=462 [M+H]⁺

¹H-NMR (400 MHz, CHLOROFORM-d) δ [ppm]: −0.069 (0.59), 0.765 (0.94), 0.812 (1.30), 0.828 (0.64), 0.885 (0.55), 0.911 (6.80), 0.930 (16.00), 0.948 (7.37), 1.077 (5.09), 1.081 (5.46), 1.095 (10.33), 1.099 (10.96), 1.113 (5.35), 1.118 (5.53), 1.164 (0.77), 1.184 (6.45), 1.216 (1.85), 1.265 (1.36), 1.268 (1.88), 1.354 (1.01), 1.440 (0.52), 1.457 (0.58), 1.609 (0.91), 1.626 (1.85), 1.642 (1.50), 1.833 (0.75), 1.848 (0.98), 1.868 (1.63), 1.883 (1.34), 1.886 (1.37), 1.901 (1.16), 1.919 (0.40), 1.956 (1.86), 1.974 (2.99), 1.991 (2.49), 2.008 (1.60), 2.026 (1.06), 2.098 (0.54), 2.117 (1.10), 2.129 (1.39), 2.148 (1.30), 2.160 (0.78), 2.321 (3.45), 2.331 (0.76), 2.386 (0.62), 2.413 (0.42), 2.430 (0.77), 2.450 (1.08), 2.463 (1.79), 2.480 (0.83), 2.485 (0.77), 2.505 (0.76), 2.509 (0.82), 2.526 (1.78), 2.540 (1.17), 2.544 (1.01), 2.554 (0.79), 2.559 (0.72), 2.573 (0.40), 2.577 (0.44), 3.205 (0.71), 3.210 (0.80), 3.219 (1.00), 3.224 (2.69), 3.228 (2.39), 3.237 (2.53), 3.241 (4.04), 3.246 (2.48), 3.255 (2.25), 3.260 (2.78), 3.273 (0.76), 3.278 (0.70), 3.401 (0.96), 3.420 (2.74), 3.426 (2.53), 3.439 (3.58), 3.455 (3.47), 3.479 (1.75), 3.514 (0.81), 3.534 (1.13), 3.547 (1.01), 3.571 (0.46), 4.073 (1.42), 4.130 (3.78), 4.148 (6.01), 4.165 (3.45), 4.737 (4.61), 5.117 (1.21), 5.131 (2.35), 5.149 (1.13), 5.233 (0.67), 5.947 (7.19), 5.959 (7.77), 6.929 (1.05), 7.123 (2.73), 7.130 (3.48), 7.134 (2.85), 7.166 (0.93), 7.170 (1.07), 7.241 (1.77), 7.261 (6.41), 7.278 (14.00), 7.294 (1.59), 7.299 (0.83), 7.451 (1.03), 7.603 (0.44), 7.608 (0.47), 7.887 (3.86), 7.891 (5.00), 7.894 (3.69), 8.029 (0.57), 8.192 (0.76).

### Example 647

### (3R)-2′-[6-amino-5-(1-phenylpropoxy)pyridin-3-yl]-N-ethyl-5′,6′-dihydro-1H-spiro[pyrrolidine-3,4′-pyrrolo[1,2-b]pyrazole]-1-carboxamide (Diastereomer 1)

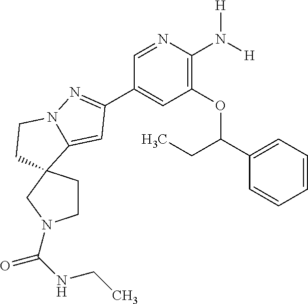 [SMILES: CCNC(=O)N1CC[C@@]2(CCn3nc(-c4cnc(N)c(OC(CC)c5ccccc5)c4)cc32)C1]

The title compound from Example 646 was separated into diastereomers by preparative chiral HPLC to give 4.00 mg (90% purity, 43% yield) of the title compound (diastereomer 1, Rₜ=11.9-13.4 min)

Preparative Chiral HPLC Method: MTBE

Instrument: PrepCon Labomatic HPLC-2; Column: YMC Cellulose SB 5μ, 250×30; eluent A: methyl tert-butyl ether+0.1 vol % diethylamine; eluent B: acetonitrile; isocratic: 50% A+50% B; flow: 50 mL/min; temperature: 25° C.; UV: 254 nm

Analytical Chiral HPLC Method: MTBE

Instrument: Thermo Fisher UltiMate 3000; Column: YMC Cellulose SB 3μ, 100×4.6; eluent A: methyl tert-butyl ether+0.1 vol % diethylamine; eluent B: acetonitrile; isocratic: 50% A+50% B; flow: 1.4 mL/min; temperature: 25° C.; UV: 254 nm

Analytical chiral HPLC: Rₜ=4.81 min.

LC-MS (Method 1): Rₜ=1.05 min; MS (ESIpos): m/z=462 [M+H]⁺

¹H-NMR (400 MHz, CHLOROFORM-d) δ [ppm]: 0.759 (1.09), 0.793 (1.09), 0.810 (1.72), 0.826 (0.87), 0.912 (5.34), 0.930 (12.27), 0.949 (5.62), 1.076 (7.20), 1.093 (16.00), 1.112 (7.54), 1.185 (7.41), 1.206 (2.06), 1.220 (1.82), 1.224 (1.43), 1.231 (1.42), 1.236 (1.60), 1.261 (0.80), 1.347 (0.55), 1.360 (0.53), 1.833 (0.66), 1.848 (0.83), 1.868 (1.35), 1.883 (1.14), 1.886 (1.12), 1.901 (1.01), 1.942 (5.28), 1.955 (1.58), 1.974 (2.16), 1.984 (1.51), 1.991 (1.75), 1.997 (1.24), 2.008 (1.37), 2.015 (0.94), 2.027 (0.71), 2.107 (0.83), 2.127 (1.44), 2.139 (0.74), 2.145 (0.92), 2.157 (1.05), 2.176 (0.56), 2.416 (0.51), 2.434 (0.92), 2.449 (1.30), 2.468 (1.57), 2.483 (1.03), 2.503 (0.99), 2.520 (1.70), 2.539 (1.27), 2.553 (1.02), 2.571 (0.56), 3.201 (0.99), 3.215 (1.36), 3.219 (3.13), 3.233 (3.46), 3.237 (3.31), 3.250 (3.07), 3.269 (0.95), 3.391 (1.03), 3.415 (3.47), 3.436 (4.58), 3.458 (2.04), 3.476 (0.70), 3.510 (0.69), 3.529 (1.01), 3.543 (0.85), 3.638 (5.36), 4.081 (0.85), 4.094 (1.46), 4.107 (0.85), 4.127 (2.73), 4.144 (4.85), 4.162 (2.33), 4.809 (2.67), 5.110 (1.46), 5.128 (2.03), 5.142 (1.37), 5.947 (10.52), 6.931 (0.47), 7.123 (3.85), 7.127 (3.86), 7.172 (0.82), 7.203 (2.51), 7.209 (1.33), 7.243 (1.45), 7.263 (5.60), 7.274 (6.56), 7.280 (14.82), 7.292 (1.43), 7.454 (0.53), 7.867 (2.33).

### Example 648

### (3R)-2′-[6-amino-5-(1-phenylpropoxy)pyridin-3-yl]-N-ethyl-5′,6′-dihydro-1H-spiro[pyrrolidine-3,4′-pyrrolo[1,2-b]pyrazole]-1-carboxamide (Diastereomer 2)

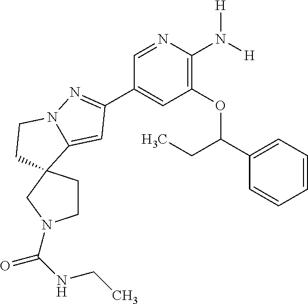 [SMILES: CCNC(=O)N1CC[C@@]2(CCn3nc(-c4cnc(N)c(OC(CC)c5ccccc5)c4)cc32)C1]

For the preparation of the diastereomeric mixture of the title compound see Example 646. The diastereomers were separated by preparative chiral HPLC (method see Example 647) to give 2.90 mg (90% purity, 65% yield) of the title compound (diastereomer 2, Rₜ=22.8-25.3 min).

Analytical chiral HPLC (method see Example 647): Rₜ=7.07 min

LC-MS (Method 1): Rₜ=1.05 min; MS (ESIpos): m/z=462 [M+H]⁺

¹H-NMR (400 MHz, CHLOROFORM-d) δ [ppm]: 0.018 (0.47), 0.740 (1.06), 0.759 (1.48), 0.765 (1.46), 0.772 (1.41), 0.783 (1.44), 0.788 (1.39), 0.811 (1.87), 0.826 (1.03), 0.852 (0.43), 0.869 (0.45), 0.910 (5.00), 0.928 (11.55), 0.947 (5.45), 1.081 (7.10), 1.099 (16.00), 1.117 (7.36), 1.185 (8.23), 1.220 (1.43), 1.225 (1.56), 1.231 (1.71), 1.349 (0.56), 1.361 (0.69), 1.387 (0.80), 1.398 (0.54), 1.403 (0.88), 1.416 (0.83), 1.435 (0.52), 1.532 (0.74), 1.834 (0.64), 1.848 (0.80), 1.869 (1.27), 1.883 (1.10), 1.887 (1.10), 1.902 (0.96), 1.940 (0.85), 1.957 (1.74), 1.975 (2.14), 1.984 (1.18), 1.993 (1.84), 2.002 (0.85), 2.010 (1.17), 2.028 (0.65), 2.093 (0.69), 2.112 (1.41), 2.124 (0.67), 2.131 (0.86), 2.143 (0.95), 2.162 (0.50), 2.411 (0.47), 2.427 (0.91), 2.443 (1.21), 2.460 (2.05), 2.477 (0.95), 2.506 (0.92), 2.524 (2.08), 2.541 (1.15), 2.557 (0.90), 2.574 (0.51), 3.186 (1.41), 3.208 (0.98), 3.222 (1.32), 3.226 (3.00), 3.240 (3.33), 3.245 (3.16), 3.258 (2.97), 3.262 (1.23), 3.276 (0.94), 3.400 (1.10), 3.412 (0.64), 3.425 (3.12), 3.449 (3.59), 3.473 (1.62), 3.507 (0.64), 3.521 (0.82), 3.527 (0.95), 3.540 (0.83), 3.638 (0.66), 4.073 (0.62), 4.092 (1.24), 4.126 (2.92), 4.143 (5.12), 4.161 (2.68), 5.119 (1.37), 5.136 (1.96), 5.151 (1.32), 5.951 (6.24), 6.929 (0.43), 7.147 (3.10), 7.162 (0.58), 7.168 (0.76), 7.176 (0.84), 7.184 (2.11), 7.199 (2.49), 7.205 (1.34), 7.239 (1.30), 7.245 (0.82), 7.255 (1.21), 7.259 (5.59), 7.270 (6.17), 7.275 (14.51), 7.290 (1.18), 7.451 (0.45), 7.863 (1.45).

### Example 649

### (3R)-2′-{6-amino-5-[(1S)-1-(3-cyanophenyl)ethoxy]pyridin-3-yl}-N-ethyl-5′,6′-dihydrospiro[pyrrolidine-3,4′-pyrrolo[1,2-b]pyrazole]-1-carboxamide

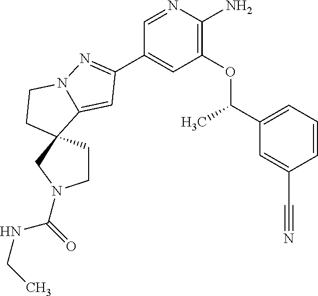 [SMILES: CCNC(=O)N1CC[C@@]2(CCn3nc(-c4cnc(N)c(O[C@@H](C)c5cccc(C#N)c5)c4)cc32)C1]

The compound was prepared similarly to Example 643 using (3R)-1-(ethylcarbamoyl)-5′,6′-dihydrospiro[pyrrolidine-3,4′-pyrrolo[1,2-b]pyrazol]-2′-yl trifluoromethanesulfonate (40.0 mg, 105 μmol) and {6-amino-5-[(1S)-1-(3-cyanophenyl)ethoxy]pyridin-3-yl}boronic acid (Intermediate 453, 101 mg, 41% purity, 146 μmol) to give 29.3 mg (98% purity, 58% yield) of the title compound.

LC-MS (Method 1): Rₜ=0.92 min; MS (ESIpos): m/z=473 [M+H]⁺

¹H-NMR (400 MHz, CHLOROFORM-d) δ [ppm]: 0.007 (0.49), 1.157 (7.09), 1.175 (16.00), 1.193 (7.29), 1.250 (1.18), 1.260 (0.41), 1.679 (10.94), 1.695 (11.08), 2.035 (0.48), 2.049 (0.65), 2.053 (0.68), 2.066 (1.16), 2.080 (0.98), 2.084 (0.89), 2.099 (0.71), 2.189 (0.70), 2.208 (1.34), 2.220 (0.68), 2.225 (0.91), 2.238 (0.89), 2.256 (0.52), 2.507 (0.51), 2.524 (1.14), 2.539 (1.29), 2.557 (2.09), 2.574 (1.00), 2.601 (1.03), 2.636 (1.27), 2.651 (1.19), 2.669 (0.56), 3.285 (1.01), 3.299 (1.37), 3.303 (3.09), 3.316 (3.46), 3.321 (3.28), 3.335 (3.19), 3.338 (1.20), 3.352 (0.97), 3.491 (1.29), 3.500 (0.64), 3.515 (3.35), 3.524 (1.13), 3.546 (4.16), 3.560 (0.82), 3.570 (1.60), 3.593 (0.69), 3.608 (0.87), 3.613 (1.01), 3.627 (0.85), 3.632 (0.71), 3.637 (0.58), 4.172 (0.80), 4.186 (1.45), 4.199 (0.83), 4.216 (3.47), 4.234 (6.68), 4.252 (3.21), 4.789 (5.06), 5.309 (10.53), 5.472 (0.61), 5.488 (1.99), 5.503 (1.96), 5.520 (0.59), 6.074 (0.45), 6.085 (11.76), 7.226 (3.86), 7.230 (3.90), 7.459 (1.44), 7.479 (3.75), 7.499 (2.59), 7.570 (1.57), 7.574 (2.94), 7.577 (2.02), 7.590 (1.24), 7.593 (2.01), 7.597 (1.32), 7.624 (1.43), 7.628 (2.32), 7.631 (1.49), 7.644 (1.12), 7.648 (1.81), 7.694 (2.33), 7.699 (3.86), 7.999 (5.20), 8.003 (5.23).

### Example 650

### (3R)-2′-{6-amino-5-[(1S)-1-(4-cyanophenyl)ethoxy]pyridin-3-yl}-N-ethyl-5′,6′-dihydrospiro[pyrrolidine-3,4′-pyrrolo[1,2-b]pyrazole]-1-carboxamide

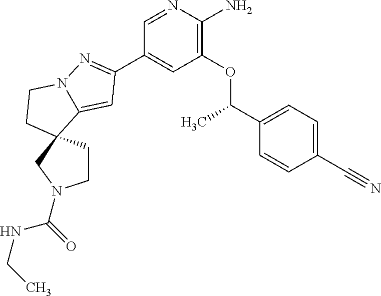 [SMILES: CCNC(=O)N1CC[C@@]2(CCn3nc(-c4cnc(N)c(O[C@@H](C)c5ccc(C#N)cc5)c4)cc32)C1]

The compound was prepared similarly to Example 643 using (3R)-1-(ethylcarbamoyl)-5′,6′-dihydrospiro[pyrrolidine-3,4′-pyrrolo[1,2-b]pyrazol]-2′-yl trifluoromethanesulfonate (40.0 mg, 105 μmol) and {6-amino-5-[(1S)-1-(4-cyanophenyl)ethoxy]pyridin-3-yl}boronic acid (Intermediate 454, 104 mg, 40% purity, 146 μmol) to give 33.5 mg (95% purity, 65% yield) of the title compound.

LC-MS (Method 1): Rₜ=0.91 min; MS (ESIpos): m/z=473 [M+H]⁺

¹H-NMR (400 MHz, CHLOROFORM-d) δ [ppm]: 0.007 (0.63), 1.156 (7.16), 1.174 (15.89), 1.192 (7.24), 1.250 (0.91), 1.678 (10.97), 1.695 (10.76), 2.030 (0.48), 2.044 (0.67), 2.048 (0.67), 2.061 (1.18), 2.075 (1.00), 2.079 (0.89), 2.094 (0.68), 2.182 (0.78), 2.201 (1.33), 2.213 (0.71), 2.218 (0.89), 2.231 (0.88), 2.250 (0.50), 2.502 (0.52), 2.519 (1.08), 2.535 (1.31), 2.552 (2.20), 2.569 (0.99), 2.597 (1.01), 2.633 (1.25), 2.648 (1.05), 2.665 (0.52), 3.285 (1.03), 3.299 (1.48), 3.303 (3.20), 3.317 (3.43), 3.321 (3.23), 3.335 (3.25), 3.339 (1.15), 3.352 (0.91), 3.488 (1.28), 3.499 (0.77), 3.513 (3.26), 3.523 (1.15), 3.541 (5.10), 3.559 (0.85), 3.565 (1.56), 3.587 (0.70), 3.602 (0.89), 3.607 (1.02), 3.621 (0.83), 3.631 (0.55), 4.156 (0.86), 4.170 (1.50), 4.183 (0.80), 4.205 (3.40), 4.223 (6.31), 4.240 (3.16), 4.789 (5.31), 5.309 (16.00), 5.485 (0.61), 5.502 (1.97), 5.517 (1.95), 5.533 (0.56), 6.054 (0.45), 6.081 (10.96), 7.191 (3.89), 7.195 (3.90), 7.494 (5.07), 7.510 (2.16), 7.514 (6.79), 7.647 (7.30), 7.651 (2.53), 7.663 (2.02), 7.668 (5.56), 7.671 (1.11), 7.996 (5.13), 8.001 (4.98).

### Example 651

### (3R)-2′-{6-amino-5-[(1S)-1-(3,5-difluoropyridin-4-yl)ethoxy]pyridin-3-yl}-N-ethyl-5′,6′-dihydrospiro[pyrrolidine-3,4′-pyrrolo[1,2-b]pyrazole]-1-carboxamide

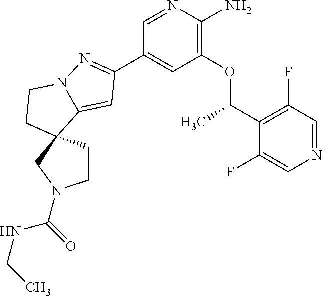 [SMILES: CCNC(=O)N1CC[C@@]2(CCn3nc(-c4cnc(N)c(O[C@@H](C)c5c(F)cncc5F)c4)cc32)C1]

The compound was prepared similarly to Example 643 using (3R)-1-(ethylcarbamoyl)-5′,6′-dihydrospiro[pyrrolidine-3,4′-pyrrolo[1,2-b]pyrazol]-2′-yl trifluoromethanesulfonate (40.0 mg, 105 μmol) and {6-amino-5-[(1S)-1-(3,5-difluoropyridin-4-yl)ethoxy]pyridin-3-yl}boronic acid (Intermediate 455, 105 mg, 41% purity, 146 μmol) to give 31.7 mg (98% purity, 61% yield) of the title compound.

[α]²⁰_{D}: −89.6° (c=1.00, MeOH)

LC-MS (Method 1): Rₜ=0.86 min; MS (ESIpos): m/z=485 [M+H]⁺

¹H-NMR (400 MHz, CHLOROFORM-d) δ [ppm]: 0.007 (0.42), 1.163 (6.51), 1.181 (14.06), 1.199 (6.70), 1.250 (0.72), 1.814 (9.26), 1.830 (9.25), 2.056 (0.50), 2.069 (0.68), 2.073 (0.70), 2.087 (1.27), 2.101 (1.01), 2.118 (0.72), 2.215 (0.73), 2.234 (1.46), 2.246 (0.71), 2.252 (0.93), 2.265 (1.02), 2.283 (0.53), 2.525 (0.48), 2.543 (1.11), 2.558 (1.32), 2.576 (1.83), 2.592 (1.02), 2.634 (2.01), 2.652 (1.27), 2.667 (1.19), 2.684 (0.54), 3.294 (0.96), 3.307 (1.39), 3.311 (2.99), 3.325 (3.35), 3.330 (3.19), 3.343 (2.98), 3.361 (0.94), 3.509 (1.48), 3.533 (3.70), 3.555 (1.81), 3.565 (3.87), 3.589 (1.60), 3.615 (0.71), 3.628 (0.88), 3.634 (1.04), 3.647 (0.90), 3.659 (0.59), 3.671 (0.43), 4.165 (0.85), 4.179 (1.50), 4.192 (0.81), 4.244 (3.24), 4.262 (5.97), 4.280 (3.08), 4.787 (5.26), 5.310 (13.56), 5.874 (0.66), 5.890 (2.12), 5.907 (2.14), 5.923 (0.64), 6.128 (10.25), 7.407 (3.90), 7.411 (3.90), 8.039 (4.91), 8.043 (4.82), 8.345 (16.00).

### Example 652

### (3′R)-2-[6-amino-5-(trifluoromethyl)pyridin-3-yl]-N-[1-(1,3,4-trimethyl-1H-pyrazol-5-yl)ethyl]-6,7-dihydro-1′H-spiro[pyrazolo[5,1-c][1,4]oxazine-4,3′-pyrrolidine]-1′-carboxamide (Diastereomer 1 and Diastereomer 2)

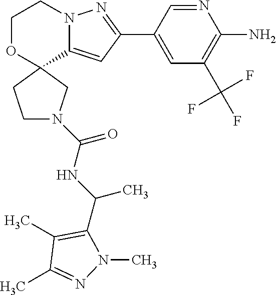 [SMILES: Cc1nn(C)c(C(C)NC(=O)N2CC[C@]3(C2)OCCn2nc(-c4cnc(N)c(C(F)(F)F)c4)cc23)c1C]

1-(1,3,4-Trimethyl-1H-pyrazol-5-yl)ethan-1-amine (47.0 mg, 307 μmol) was dissolved in DMF (2.5 mL). CDI (45.6 mg, 281 μmol) and N,N-diisopropylethylamine (180 μL, 1.0 mmol) were added and the mixture was stirred for 1 h at rt. A solution of 5-[(3′R)-6,7-dihydrospiro[pyrazolo[5,1-c][1,4]oxazine-4,3′-pyrrolidin]-2-yl]-3-(trifluoromethyl)pyridin-2-amine-hydrogen chloride (1/1) (120 mg, 80% purity, 255 μmol) in DMF was added dropwise to the reaction mixture and stirred for 1 h at 60° C. The reaction mixture was allowed to cool down, diluted with water and extracted 3 times with ethyl acetate. The combined organic layers were dried over a hydrophobic filter and concentrated. The crude product was purified by flash chromatography (silica gel, dichloromethane/methanol (0-15%) gradient) to give 125 mg (95% purity, 89% yield) of the title compound.

LC-MS (Method 1): Rₜ=0.92 min; MS (ESIpos): m/z=519 [M+H]⁺

¹H-NMR (400 MHz, DMSO-d6) δ [ppm]: 1.326 (4.32), 1.333 (4.39), 1.345 (4.35), 1.350 (4.11), 2.069 (0.71), 2.120 (13.15), 2.135 (0.76), 2.165 (0.82), 2.191 (15.74), 2.216 (0.95), 2.228 (0.75), 2.240 (0.60), 2.283 (0.69), 2.294 (0.74), 2.309 (0.84), 2.326 (1.00), 2.522 (1.76), 2.669 (0.55), 2.673 (0.40), 3.159 (3.08), 3.172 (3.28), 3.384 (1.72), 3.413 (1.28), 3.435 (0.86), 3.464 (0.97), 3.504 (0.50), 3.527 (0.81), 3.563 (16.00), 3.582 (0.90), 3.606 (0.43), 3.615 (0.62), 3.732 (0.78), 3.760 (0.72), 3.801 (0.76), 3.830 (0.65), 4.063 (1.00), 4.095 (1.74), 4.100 (1.81), 4.124 (3.71), 4.681 (0.75), 4.686 (0.79), 4.698 (1.10), 4.703 (1.13), 4.715 (0.76), 4.720 (0.73), 5.758 (10.38), 6.084 (0.90), 6.101 (0.84), 6.130 (0.99), 6.147 (0.88), 6.595 (4.71), 6.724 (3.72), 6.740 (3.12), 7.012 (3.67), 7.416 (0.63), 7.421 (0.66), 7.639 (2.38), 8.006 (2.64), 8.574 (2.59).

### Example 653

### (3′R)-2-[6-amino-5-(trifluoromethyl)pyridin-3-yl]-N-[1-(1,3,4-trimethyl-1H-pyrazol-5-yl)ethyl]-6,7-dihydro-1′H-spiro[pyrazolo[5,1-c][1,4]oxazine-4,3′-pyrrolidine]-1′-carboxamide (Diastereomer 1)

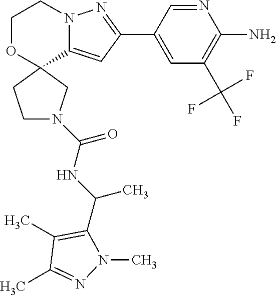 [SMILES: Cc1nn(C)c(C(C)NC(=O)N2CC[C@]3(C2)OCCn2nc(-c4cnc(N)c(C(F)(F)F)c4)cc23)c1C]

The title compound from Example 652 was separated into diastereomers by preparative chiral HPLC to give 41.0 mg (100% purity, 31% yield) of the title compound (diastereomer 1, Rₜ=8.0-10.7 min)

Preparative Chiral HPLC Method: MTBE

Instrument: PrepCon Labomatic HPLC-3; Column: Chiralpak IF 5μ, 250×30; eluent A: methyl tert-butyl ether+0.1 vol % diethylamine; eluent B: ethanol+0.1 vol % diethylamine; isocratic: 80% A+20% B; flow: 40 mL/min; temperature: 25° C.; UV: 254 nm

Analytical Chiral HPLC Method: MTBE

Instrument: Thermo Fisher UltiMate 3000; Column: Chiralpak IF 3μ, 100×4.6; eluent A: methyl tert-butyl ether+0.1 vol % diethylamine; eluent B: ethanol; isocratic: 80% A+20% B; flow: 1.4 mL/min; temperature: 25° C.; UV: 254 nm

Analytical chiral HPLC: Rₜ=2.99 min.

[α]²⁰_{D}: +37.3° (c=1.00, MeOH)

LC-MS (Method 1): Rₜ=0.92 min; MS (ESIpos): m/z=519 [M+H]⁺

¹H-NMR (400 MHz, DMSO-d6) δ [ppm]: 0.798 (1.10), 0.803 (0.47), 0.814 (1.12), 0.821 (1.13), 0.840 (0.56), 0.886 (0.56), 0.904 (1.26), 0.922 (0.65), 1.137 (0.58), 1.183 (0.95), 1.324 (1.17), 1.333 (5.01), 1.342 (1.44), 1.350 (5.07), 2.074 (0.71), 2.113 (3.31), 2.118 (13.56), 2.185 (4.16), 2.190 (15.38), 2.210 (0.64), 2.293 (0.49), 2.308 (0.57), 2.322 (0.65), 2.326 (0.82), 2.331 (0.56), 2.518 (1.85), 2.522 (1.12), 2.669 (0.52), 3.294 (0.41), 3.313 (0.86), 3.363 (0.67), 3.384 (1.44), 3.413 (1.48), 3.562 (16.00), 3.582 (0.93), 3.800 (0.99), 3.829 (0.86), 4.047 (0.63), 4.064 (0.81), 4.083 (0.97), 4.095 (1.08), 4.104 (1.08), 4.123 (2.47), 4.685 (0.86), 4.703 (1.24), 4.720 (0.83), 6.083 (1.24), 6.100 (1.16), 6.235 (0.57), 6.239 (0.58), 6.596 (3.23), 6.724 (5.93), 7.417 (0.53), 7.421 (0.52), 8.005 (1.95), 8.010 (1.97), 8.574 (1.82), 8.578 (1.79).

### Example 654

### (3′R)-2-[6-amino-5-(trifluoromethyl)pyridin-3-yl]-N-[1-(1,3,4-trimethyl-1H-pyrazol-5-yl)ethyl]-6,7-dihydro-1′H-spiro[pyrazolo[5,1-c][1,4]oxazine-4,3′-pyrrolidine]-1′-carboxamide (Diastereomer 2)

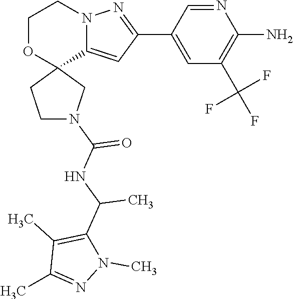 [SMILES: Cc1nn(C)c(C(C)NC(=O)N2CC[C@]3(C2)OCCn2nc(-c4cnc(N)c(C(F)(F)F)c4)cc23)c1C]

For the preparation of the diastereomeric mixture of the title compound see Example 652. The diastereomers were separated by preparative chiral HPLC (method see Example 653) to give 55.0 mg (100% purity, 41% yield) of the title compound (diastereomer 2, Rₜ=12.1-15.1 min).

Analytical chiral HPLC (method see Example 653): Rₜ=5.28 min

[α]²⁰_{D}: +62.5° (c=1.00, MeOH)

LC-MS (Method 1): Rₜ=0.92 min; MS (ESIpos): m/z=519 [M+H]⁺

¹H-NMR (400 MHz, DMSO-d6) δ [ppm]: 1.326 (5.46), 1.344 (5.53), 2.073 (0.55), 2.120 (14.81), 2.186 (3.81), 2.191 (16.00), 2.217 (0.75), 2.240 (0.46), 2.294 (0.59), 2.311 (0.68), 2.322 (0.68), 2.326 (0.90), 2.331 (0.66), 2.522 (1.60), 2.668 (0.51), 3.375 (1.03), 3.384 (0.71), 3.401 (0.45), 3.435 (1.26), 3.464 (1.49), 3.505 (0.68), 3.526 (1.08), 3.564 (14.89), 3.731 (1.19), 3.758 (0.96), 4.026 (0.42), 4.041 (0.67), 4.057 (0.94), 4.094 (1.26), 4.104 (1.24), 4.124 (2.87), 4.680 (0.93), 4.697 (1.36), 4.715 (0.92), 6.130 (1.52), 6.147 (1.43), 6.246 (0.41), 6.251 (0.43), 6.594 (3.82), 6.740 (5.02), 7.416 (0.44), 7.421 (0.43), 8.002 (2.25), 8.007 (2.28), 8.568 (2.13), 8.573 (2.11).

### Example 655

### (3′R)-2-[6-amino-5-(trifluoromethyl)pyridin-3-yl]-N-[1-(1,5-dimethyl-1H-pyrazol-4-yl)ethyl]-6,7-dihydro-1′H-spiro[pyrazolo[5,1-c][1,4]oxazine-4,3′-pyrrolidine]-1′-carboxamide (Diastereomer 1 and Diastereomer 2)

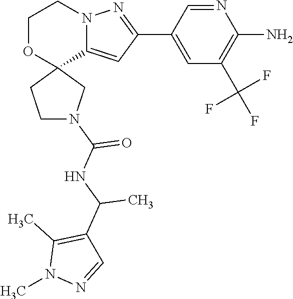 [SMILES: Cc1c(C(C)NC(=O)N2CC[C@]3(C2)OCCn2nc(-c4cnc(N)c(C(F)(F)F)c4)cc23)cnn1C]

1-(1,5-Dimethyl-1H-pyrazol-4-yl)ethan-1-amine (20.9 mg, 150 μmol) was dissolved in DMF (1.5 mL). CDI (22.4 mg, 138 μmol) and N,N-diisopropylethylamine (87 μL, 500 μmol) were added and the mixture was stirred for 1 h at rt. A solution of 5-[(3′R)-6,7-dihydrospiro[pyrazolo[5,1-c][1,4]oxazine-4,3′-pyrrolidin]-2-yl]-3-(trifluoromethyl)pyridin-2-amine-hydrogen chloride (47.1 mg, 125 μmol) in DMF was added dropwise to the reaction mixture and stirred for 1 h at 60° C. The reaction mixture was allowed to cool down, diluted with water and extracted 3 times with ethyl acetate. The combined organic layers were dried over a hydrophobic filter and concentrated. The crude product was purified by preparative HPLC and preparative TLC to give 14.5 mg (90% purity, 21% yield) of the title compound.

LC-MS (Method 1): Rₜ=0.89 min; MS (ESIpos): m/z=505 [M+H]⁺

¹H-NMR (400 MHz, CHLOROFORM-d) δ [ppm]: 0.782 (0.64), 0.792 (0.67), 0.809 (1.17), 0.826 (0.65), 0.835 (0.47), 0.883 (0.47), 1.120 (0.55), 1.138 (1.00), 1.147 (0.95), 1.157 (1.40), 1.183 (8.92), 1.214 (1.17), 1.263 (0.60), 1.394 (0.44), 1.428 (10.80), 1.444 (10.80), 1.500 (0.40), 1.506 (0.56), 1.523 (0.60), 2.179 (0.57), 2.188 (0.48), 2.204 (12.49), 2.210 (7.48), 2.221 (0.77), 2.235 (1.18), 2.244 (0.84), 2.260 (0.58), 2.269 (0.57), 2.328 (1.21), 2.343 (0.88), 2.362 (0.74), 3.442 (0.60), 3.462 (1.92), 3.467 (1.68), 3.477 (2.35), 3.490 (2.34), 3.497 (1.67), 3.506 (2.66), 3.524 (0.88), 3.540 (0.99), 3.596 (0.46), 3.695 (13.50), 3.698 (7.98), 3.788 (0.81), 3.800 (16.00), 3.823 (0.92), 3.850 (1.34), 3.873 (0.82), 4.064 (2.83), 4.069 (3.08), 4.076 (3.53), 4.085 (2.27), 4.142 (3.01), 4.148 (3.64), 4.156 (4.63), 4.195 (0.84), 4.203 (0.73), 4.215 (0.68), 4.223 (0.61), 4.860 (0.68), 4.867 (0.44), 4.878 (0.98), 4.885 (0.56), 4.895 (0.68), 4.947 (0.77), 4.966 (1.43), 4.982 (6.90), 6.193 (5.25), 6.195 (3.30), 6.202 (4.08), 6.204 (3.68), 7.273 (3.48), 7.308 (3.68), 7.375 (2.59), 7.897 (1.68), 7.901 (1.69), 7.918 (1.67), 7.922 (1.59), 8.027 (4.09), 8.407 (2.37), 8.411 (2.29), 8.504 (3.72).

### Example 656

### (3′R)-2-[6-amino-5-(trifluoromethyl)pyridin-3-yl]-N-[1-(1-methyl-1H-pyrazol-4-yl)ethyl]-6,7-dihydro-1′H-spiro[pyrazolo[5,1-c][1,4]oxazine-4,3′-pyrrolidine]-1′-carboxamide (Diastereomer 1 and Diastereomer 2)

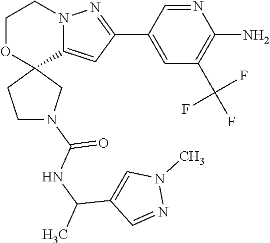 [SMILES: CC(NC(=O)N1CC[C@]2(C1)OCCn1nc(-c3cnc(N)c(C(F)(F)F)c3)cc12)c1cnn(C)c1]

The compound was prepared similarly to Example 655 using 5-[(3′R)-6,7-dihydrospiro[pyrazolo[5,1-c][1,4]oxazine-4,3′-pyrrolidin]-2-yl]-3-(trifluoromethyl)pyridin-2-amine-hydrogen chloride (47.1 mg, 125 μmol) and 1-(1-methyl-1H-pyrazol-4-yl)ethan-1-amine (18.8 mg, 150 μmol) to give 5.00 mg (90% purity, 7% yield) of the title compound.

LC-MS (Method 1): Rₜ=0.86 min; MS (ESIpos): m/z=491 [M+H]⁺

¹H-NMR (400 MHz, CHLOROFORM-d) δ [ppm]: 1.253 (2.54), 1.495 (5.91), 2.273 (2.48), 2.278 (1.77), 2.289 (0.59), 2.304 (0.57), 2.312 (0.76), 2.337 (0.55), 2.396 (0.68), 2.411 (0.68), 2.430 (0.44), 3.531 (0.55), 3.547 (2.15), 3.559 (0.86), 3.568 (0.89), 3.575 (2.43), 3.594 (0.77), 3.616 (0.69), 3.763 (2.63), 3.767 (1.49), 3.867 (16.00), 3.918 (0.87), 3.945 (0.66), 4.126 (0.98), 4.132 (1.24), 4.143 (2.31), 4.153 (2.16), 4.216 (2.38), 4.224 (2.28), 4.229 (2.30), 4.238 (1.16), 4.243 (1.00), 4.288 (0.64), 4.296 (0.68), 4.307 (0.70), 4.315 (0.65), 5.015 (0.70), 5.033 (1.00), 5.051 (0.69), 5.093 (3.98), 6.262 (1.07), 6.265 (0.75), 6.271 (4.08), 6.274 (3.76), 6.340 (0.44), 7.270 (4.85), 7.376 (0.78), 7.443 (2.93), 8.094 (2.39), 8.571 (2.26), 8.574 (2.09).

### Example 657

### (3R)-2′-{6-amino-5-[(1R)-1-(3-fluoropyridin-2-yl)ethoxy]pyridin-3-yl}-N-(propan-2-yl)-5′,6′-dihydrospiro[pyrrolidine-3,4′-pyrrolo[1,2-b]pyrazole]-1-carboxamide

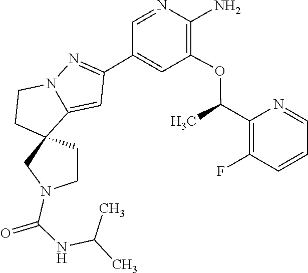 [SMILES: CC(C)NC(=O)N1CC[C@@]2(CCn3nc(-c4cnc(N)c(O[C@H](C)c5ncccc5F)c4)cc32)C1]

5-[(3R)-5′,6′-Dihydrospiro[pyrrolidine-3,4′-pyrrolo[1,2-b]pyrazol]-2′-yl]-3-[(1R)-1-(3-fluoropyridin-2-yl)ethoxy]pyridin-2-amine-hydrochloride (Intermediate 517, 70.0 mg, 35% purity, 56.9 μmol) was suspended in dichloromethane (0.9 mL). N,N-Diisopropylethylamine (99 μL, 570 μmol) was added, cooled to 0° C., isopropyl isocyanate (6.7 μL, 68 μmol) was added and the mixture was allowed to warm up to rt over 2 h. The reaction mixture was diluted with water and brine, extracted 2 times with dichloromethane. The combined organic layers were dried over sodium sulfate, filtered and concentrated. The residue was purified by preparative TLC (gradient: dichloromethane/methanol 9:1) to give 22.8 mg (98% purity, 82% yield) of the title compound.

LC-MS (Method 1): Rₜ=0.92 min; MS (ESIpos): m/z=481 [M+H]⁺

¹H-NMR (400 MHz, CHLOROFORM-d) δ [ppm]: 1.179 (11.95), 1.186 (12.83), 1.195 (13.52), 1.202 (11.04), 1.260 (0.49), 1.783 (12.69), 1.799 (12.59), 2.044 (0.58), 2.058 (0.77), 2.062 (0.79), 2.075 (1.48), 2.089 (1.23), 2.092 (1.45), 2.107 (0.83), 2.207 (0.89), 2.226 (1.81), 2.238 (0.83), 2.244 (1.10), 2.256 (1.27), 2.275 (0.66), 2.515 (0.61), 2.533 (1.40), 2.548 (1.60), 2.565 (2.11), 2.582 (1.24), 2.642 (1.61), 2.656 (1.54), 2.674 (0.69), 3.482 (1.43), 3.497 (0.87), 3.507 (3.58), 3.514 (1.83), 3.521 (1.26), 3.537 (5.74), 3.561 (2.13), 3.604 (0.82), 3.617 (0.99), 3.624 (1.16), 3.636 (1.01), 3.647 (0.68), 3.661 (0.50), 3.979 (0.86), 3.998 (3.72), 4.012 (2.08), 4.027 (1.46), 4.045 (0.70), 4.236 (4.15), 4.254 (7.39), 4.271 (3.82), 4.862 (6.21), 5.310 (14.29), 5.794 (0.81), 5.811 (2.61), 5.826 (2.57), 5.843 (0.77), 6.106 (16.00), 7.006 (0.52), 7.247 (1.65), 7.258 (2.47), 7.278 (3.32), 7.290 (2.19), 7.378 (2.22), 7.381 (2.16), 7.399 (1.85), 7.402 (3.42), 7.406 (2.38), 7.424 (1.57), 7.427 (1.66), 7.449 (4.89), 7.454 (4.86), 7.528 (0.51), 8.029 (6.68), 8.034 (6.55), 8.041 (0.41), 8.429 (1.88), 8.433 (3.39), 8.436 (2.02), 8.441 (2.01), 8.445 (3.23), 8.448 (1.84).

### Example 658

(3R)-2′-{6-amino-5-[(1R)-1-(3-fluoropyridin-2-yl)ethoxy]pyridin-3-yl}-N-cyclobutyl-5′,6′-dihydrospiro[pyrrolidine-3,4′-pyrrolo[1,2-b]pyrazole]-1-carboxamide

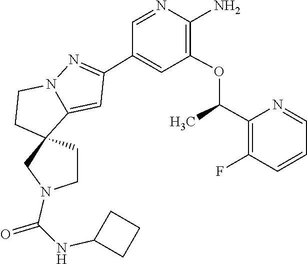 [SMILES: C[C@@H](Oc1cc(-c2cc3n(n2)CC[C@@]32CCN(C(=O)NC3CCC3)C2)cnc1N)c1ncccc1F]

5-[(3R)-5′,6′-Dihydrospiro[pyrrolidine-3,4′-pyrrolo[1,2-b]pyrazol]-2′-yl]-3-[(1R)-1-(3-fluoropyridin-2-yl)ethoxy]pyridin-2-amine-hydrochloride (Intermediate 517, 70.0 mg, 35% purity, 56.9 μmol) was suspended in dichloromethane (0.9 mL). N,N-Diisopropylethylamine (99 μL, 570 μmol) was added, cooled to 0° C., cyclobutyl isocyanate (6.5 μL, 68 μmol) was added and the mixture was allowed to warm up to rt over 2 h. The reaction mixture was diluted with water and brine, extracted 2 times with dichloromethane. The combined organic layers were dried over sodium sulfate, filtered and concentrated. The residue was purified by preparative TLC (gradient: dichloromethane/methanol 9:1) to give 23.7 mg (98% purity, 83% yield) of the title compound.

LC-MS (Method 1): Rₜ=0.94 min; MS (ESIpos): m/z=493 [M+H]⁺

¹H-NMR (400 MHz, CHLOROFORM-d) δ [ppm]: 1.260 (0.49), 1.657 (2.96), 1.666 (2.37), 1.683 (2.47), 1.688 (2.08), 1.693 (1.32), 1.698 (1.63), 1.710 (2.54), 1.721 (0.97), 1.724 (0.89), 1.783 (10.63), 1.799 (11.00), 1.816 (1.14), 1.829 (1.20), 1.838 (1.15), 1.855 (0.82), 2.042 (0.48), 2.056 (0.66), 2.060 (0.65), 2.073 (1.18), 2.087 (0.98), 2.092 (1.08), 2.105 (0.68), 2.203 (0.71), 2.222 (1.42), 2.234 (0.67), 2.240 (0.87), 2.253 (0.99), 2.272 (0.52), 2.340 (0.59), 2.349 (0.75), 2.358 (1.25), 2.368 (1.46), 2.377 (1.43), 2.382 (1.25), 2.386 (1.31), 2.393 (1.01), 2.510 (0.48), 2.527 (1.08), 2.543 (1.27), 2.560 (1.64), 2.577 (1.00), 2.600 (1.04), 2.636 (1.29), 2.650 (1.23), 2.668 (0.56), 3.485 (1.07), 3.510 (2.81), 3.519 (1.31), 3.526 (1.08), 3.535 (4.22), 3.544 (1.77), 3.560 (1.91), 3.605 (0.65), 3.618 (0.79), 3.624 (0.94), 3.637 (0.81), 3.648 (0.54), 4.233 (3.17), 4.251 (5.66), 4.268 (2.95), 4.313 (0.42), 4.334 (1.57), 4.345 (3.17), 4.360 (1.01), 4.380 (0.46), 4.862 (5.13), 5.310 (16.00), 5.794 (0.66), 5.810 (2.12), 5.826 (2.11), 5.843 (0.64), 6.104 (11.79), 7.006 (0.43), 7.247 (1.33), 7.258 (2.03), 7.279 (2.74), 7.290 (1.72), 7.378 (1.75), 7.381 (1.87), 7.399 (1.44), 7.402 (2.66), 7.405 (1.85), 7.423 (1.39), 7.426 (1.32), 7.448 (3.95), 7.452 (4.00), 7.528 (0.44), 8.029 (5.33), 8.033 (5.23), 8.429 (1.54), 8.432 (2.70), 8.436 (1.63), 8.440 (1.63), 8.444 (2.68), 8.447 (1.47).

### Example 659

### (3R)-2′-{6-amino-5-[(1R)-1-(3,5-difluoropyridin-4-yl)ethoxy]pyridin-3-yl}-N-(propan-2-yl)-5′,6′-dihydrospiro[pyrrolidine-3,4′-pyrrolo[1,2-b]pyrazole]-1-carboxamide

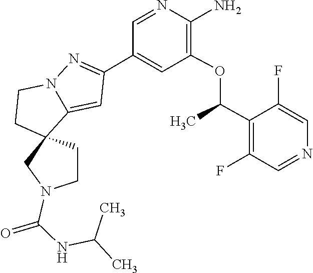 [SMILES: CC(C)NC(=O)N1CC[C@@]2(CCn3nc(-c4cnc(N)c(O[C@H](C)c5c(F)cncc5F)c4)cc32)C1]

3-[(1R)-1-(3,5-Difluoropyridin-4-yl)ethoxy]-5-[(3R)-5′,6′-dihydrospiro[pyrrolidine-3,4′-pyrrolo[1,2-b]pyrazol]-2′-yl]pyridin-2-amine-hydrochloride (Intermediate 518, 70.0 mg, 40% purity, 62.4 μmol) was suspended in dichloromethane (1.1 mL). N,N-Diisopropylethylamine (110 μL, 620 μmol) was added, cooled to 0° C. and isopropyl isocyanate (7.4 μL, 75 μmol) was added. The mixture was allowed to warm up to rt over 2 h. The reaction mixture was diluted with water and brine, extracted 2 times with dichloromethane. The combined organic layers were dried over sodium sulfate, filtered and concentrated. The residue was purified by preparative TLC (gradient: dichloromethane/methanol 9:1) to give 21.6 mg (98% purity, 68% yield) of the title compound.

LC-MS (Method 1): Rₜ=0.94 min; MS (ESIpos): m/z=499 [M+H]⁺

¹H-NMR (400 MHz, CHLOROFORM-d) δ [ppm]: 1.177 (8.84), 1.186 (9.61), 1.192 (10.12), 1.201 (8.14), 1.261 (0.51), 1.814 (8.57), 1.831 (8.54), 2.055 (0.44), 2.068 (0.61), 2.073 (0.61), 2.086 (1.12), 2.099 (1.20), 2.117 (0.63), 2.218 (0.65), 2.237 (1.35), 2.250 (0.64), 2.256 (0.82), 2.269 (0.93), 2.287 (0.48), 2.526 (0.47), 2.543 (1.09), 2.559 (1.17), 2.576 (1.76), 2.593 (0.94), 2.637 (1.95), 2.655 (1.19), 2.669 (1.13), 2.687 (0.50), 3.491 (1.13), 3.503 (0.61), 3.516 (2.81), 3.527 (0.95), 3.545 (4.74), 3.563 (0.80), 3.570 (1.47), 3.610 (0.59), 3.622 (0.75), 3.628 (0.86), 3.642 (0.77), 3.652 (0.51), 3.979 (0.66), 3.997 (2.81), 4.011 (1.55), 4.027 (1.09), 4.045 (0.52), 4.245 (3.13), 4.263 (5.91), 4.281 (2.88), 4.787 (4.79), 5.310 (7.35), 5.874 (0.60), 5.890 (1.94), 5.907 (1.93), 5.923 (0.57), 6.123 (10.85), 7.006 (0.52), 7.401 (3.54), 7.405 (3.58), 7.528 (0.51), 8.041 (4.89), 8.044 (4.79), 8.346 (16.00).

### Example 660

### (3R)-2′-{6-amino-5-[(1R)-1-(3,5-difluoropyridin-4-yl)ethoxy]pyridin-3-yl}-N-cyclobutyl-5′,6′-dihydrospiro[pyrrolidine-3,4′-pyrrolo[1,2-b]pyrazole]-1-carboxamide

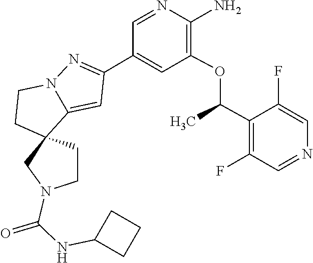 [SMILES: C[C@@H](Oc1cc(-c2cc3n(n2)CC[C@@]32CCN(C(=O)NC3CCC3)C2)cnc1N)c1c(F)cncc1F]

3-[(1R)-1-(3,5-Difluoropyridin-4-yl)ethoxy]-5-[(3R)-5′,6′-dihydrospiro[pyrrolidine-3,4′-pyrrolo[1,2-b]pyrazol]-2′-yl]pyridin-2-amine-hydrochloride (Intermediate 518, 70.0 mg, 40% purity, 62.4 μmol) was suspended in dichloromethane (1.1 mL). N,N-Diisopropylethylamine (110 μL, 620 μmol) was added, cooled to 0° C., cyclobutyl isocyanate (7.1 μL, 75 μmol) was added and the mixture was allowed to warm up to rt over 2 h. The reaction mixture was diluted with water and brine, extracted 2 times with dichloromethane. The combined organic layers were dried over sodium sulfate, filtered and concentrated. The residue was purified by preparative TLC (gradient: dichloromethane/methanol 9:1) to give 24.0 mg (98% purity, 74% yield) of the title compound.

LC-MS (Method 1): Rₜ=0.97 min; MS (ESIpos): m/z=511 [M+H]⁺

¹H-NMR (400 MHz, CHLOROFORM-d) δ [ppm]: 1.261 (0.42), 1.640 (1.03), 1.657 (1.22), 1.666 (1.54), 1.683 (2.03), 1.688 (1.77), 1.693 (1.11), 1.698 (1.46), 1.710 (2.28), 1.720 (0.89), 1.723 (0.79), 1.795 (0.83), 1.814 (9.67), 1.831 (9.58), 1.851 (0.81), 2.053 (0.43), 2.066 (0.59), 2.071 (0.61), 2.084 (1.11), 2.097 (1.01), 2.115 (0.62), 2.214 (0.64), 2.233 (1.29), 2.246 (0.61), 2.251 (0.80), 2.264 (0.92), 2.283 (0.48), 2.339 (0.54), 2.347 (0.73), 2.356 (1.19), 2.366 (1.41), 2.376 (1.31), 2.380 (1.26), 2.384 (1.21), 2.521 (0.46), 2.539 (1.03), 2.554 (1.17), 2.571 (1.65), 2.588 (0.92), 2.630 (1.87), 2.648 (1.16), 2.663 (1.14), 2.681 (0.51), 3.494 (1.01), 3.518 (2.70), 3.525 (1.33), 3.532 (1.00), 3.544 (4.07), 3.549 (1.81), 3.568 (1.89), 3.610 (0.60), 3.624 (0.75), 3.630 (0.87), 3.643 (0.76), 3.654 (0.50), 4.243 (3.05), 4.261 (5.63), 4.278 (2.80), 4.333 (1.53), 4.344 (2.98), 4.360 (0.94), 4.379 (0.45), 4.783 (4.95), 5.310 (7.83), 5.874 (0.60), 5.890 (1.95), 5.907 (1.95), 5.923 (0.59), 6.121 (10.39), 7.006 (0.42), 7.400 (3.58), 7.404 (3.64), 7.528 (0.43), 8.040 (4.86), 8.044 (4.91), 8.346 (16.00).

### Example 661

### (3R)-2′-{6-amino-5-[(1R)-1-phenylethoxy]pyridin-3-yl}-N-ethyl-5′,6′-dihydrospiro[pyrrolidine-3,4′-pyrrolo[1,2-b]pyrazole]-1-carboxamide

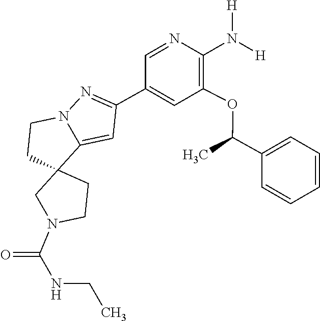 [SMILES: CCNC(=O)N1CC[C@@]2(CCn3nc(-c4cnc(N)c(O[C@H](C)c5ccccc5)c4)cc32)C1]

(3R)-1-(Ethylcarbamoyl)-5′,6′-dihydrospiro[pyrrolidine-3,4′-pyrrolo[1,2-b]pyrazol]-2′-yl trifluoromethanesulfonate (117 mg, 305 μmol), {6-amino-5-[(1R)-1-phenylethoxy]pyridin-3-yl}boronic acid (Intermediate 456, 397 mg, 22% purity, 335 μmol) and potassium phosphate (1.8 mL, 0.50 M, 910 μmol) were suspended in degassed 1,4-dioxane (3.4 mL) under nitrogen. XPhos Pd G2 (12.0 mg, 15.2 μmol) was added and the mixture was stirred for 2 h at 100° C. The reaction mixture was allowed to cool down, diluted with ethyl acetate and filtered. The filtrate was washed with water and brine. The organic phase was dried over a hydrophobic filter and concentrated. The crude product was purified by flash column chromatography (silica gel, dichloromethane/methanol 9:1 gradient) to give 20.1 mg (90% purity, 13% yield) of the title compound.

LC-MS (Method 1): Rₜ=0.98 min; MS (ESIpos): m/z=448 [M+H]⁺

¹H-NMR (400 MHz, CHLOROFORM-d) δ [ppm]: 1.078 (7.35), 1.096 (16.00), 1.114 (7.54), 1.173 (0.42), 1.183 (0.96), 1.191 (0.66), 1.204 (0.78), 1.601 (12.99), 1.617 (12.74), 1.959 (0.58), 1.973 (0.79), 1.978 (1.31), 1.990 (1.40), 2.003 (1.13), 2.008 (1.03), 2.022 (0.83), 2.115 (0.82), 2.134 (1.57), 2.147 (0.79), 2.153 (1.04), 2.165 (1.10), 2.184 (0.60), 2.424 (0.56), 2.442 (1.04), 2.457 (1.47), 2.475 (1.70), 2.492 (1.12), 2.513 (1.13), 2.529 (1.83), 2.548 (1.41), 2.563 (1.14), 2.580 (0.58), 3.207 (1.08), 3.220 (1.48), 3.224 (3.35), 3.238 (3.67), 3.243 (3.55), 3.256 (3.29), 3.260 (1.46), 3.274 (1.07), 3.403 (1.26), 3.427 (3.56), 3.441 (1.51), 3.451 (4.70), 3.465 (1.95), 3.476 (1.73), 3.483 (0.89), 3.519 (0.81), 3.533 (0.98), 3.538 (1.14), 3.552 (1.03), 3.562 (0.68), 3.576 (0.50), 4.075 (0.94), 4.089 (1.62), 4.102 (0.91), 4.138 (2.95), 4.156 (5.23), 4.173 (2.60), 4.764 (3.48), 5.232 (12.88), 5.366 (0.72), 5.382 (2.34), 5.398 (2.35), 5.414 (0.72), 5.980 (10.63), 7.182 (0.86), 7.187 (1.66), 7.200 (5.69), 7.210 (1.33), 7.217 (1.39), 7.222 (2.37), 7.225 (1.49), 7.261 (2.49), 7.277 (2.36), 7.281 (6.08), 7.294 (1.29), 7.299 (4.81), 7.309 (5.63), 7.313 (6.53), 7.330 (2.69), 7.334 (1.92), 7.906 (4.50), 7.909 (4.42).

### Example 662

### (3R)-2′-{6-amino-5-[1-(1-methyl-1H-pyrazol-5-yl)ethoxy]pyridin-3-yl}-N-isopropyl-5′,6′-dihydro-1H-spiro[pyrrolidine-3,4′-pyrrolo[1,2-b]pyrazole]-1-carboxamide (Diastereomer 1)

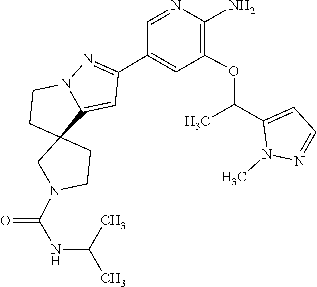 [SMILES: CC(C)NC(=O)N1CC[C@@]2(CCn3nc(-c4cnc(N)c(OC(C)c5ccnn5C)c4)cc32)C1]

5-[(3R)-5′,6′-Dihydrospiro[pyrrolidine-3,4′-pyrrolo[1,2-b]pyrazol]-2′-yl]-3-[1-(1-methyl-1H-pyrazol-5-yl)ethoxy]pyridin-2-amine-hydrochloride (Intermediate 519, 60.0 mg, 80% purity, 115 μmol) was suspended in dichloromethane (2.0 mL). N,N-Diisopropylethylamine (200 μL, 1.2 mmol) was added and cooled to 0° C. Isopropyl isocyanate (14 μL, 140 μmol) was added and the mixture was allowed to warm up to rt over 2 h. The reaction mixture was concentrated and purified by column chromatography (silica gel, dichloromethane/methanol (0-20%) gradient). The combined fractions were diluted with water and brine and extracted 2 times with ethyl acetate. The organic phase was dried over sodium sulfate, filtered and concentrated to give 41.8 mg (78% yield) of the title compound as a diastereomeric mixture.

The diastereomers were separated by preparative chiral HPLC to give 14.2 mg (95% purity, 32% yield) of the title compound (diastereomer 1, Rₜ=6.4-7.0 min)

Preparative Chiral HPLC Method:

Instrument: PrepCon Labomatic HPLC-2; Column: YMC Cellulose SB 5μ, 250×30; eluent A: methyl tert-butyl ether+0.1 vol % diethylamine; eluent B: ethanol+0.1 vol % diethylamine; isocratic: 80% A+20% B; flow: 40 mL/min; temperature: 25° C.; UV: 280 nm

Analytical Chiral HPLC Method:

Instrument: Thermo Fisher UltiMate 3000; Column: YMC Cellulose SB 3μ, 100×4.6; eluent A: methyl tert-butyl ether+0.1 vol % diethylamine; eluent B: ethanol; isocratic: 80% A+20% B; flow: 1.4 mL/min; temperature: 25° C.; UV: 280 nm

Analytical chiral HPLC: Rₜ=3.26 min.

[α]²⁰_{D}: +82.6° (c=1.00, MeOH)

LC-MS (Method 1): Rₜ=0.84 min; MS (ESIpos): m/z=466 [M+H]⁺

¹H-NMR (400 MHz, CHLOROFORM-d) δ [ppm]: 1.180 (3.98), 1.188 (4.46), 1.195 (4.79), 1.204 (3.76), 1.263 (1.54), 1.759 (5.65), 1.775 (5.57), 2.092 (0.54), 2.105 (0.43), 2.241 (0.60), 2.271 (0.41), 2.553 (0.51), 2.568 (0.56), 2.585 (0.78), 2.602 (0.44), 2.630 (0.45), 2.647 (0.87), 2.665 (0.55), 2.680 (0.54), 3.499 (0.55), 3.523 (1.44), 3.532 (0.47), 3.554 (1.80), 3.578 (0.68), 3.632 (0.41), 3.870 (16.00), 4.001 (1.24), 4.013 (0.80), 4.028 (0.55), 4.251 (1.44), 4.268 (2.61), 4.286 (1.34), 4.822 (0.44), 5.310 (6.55), 5.568 (0.93), 5.583 (0.92), 6.162 (4.78), 6.337 (1.97), 6.342 (1.98), 7.448 (2.29), 7.453 (3.56), 7.457 (1.70), 8.035 (1.19), 8.039 (1.17).

### Example 663

### (3R)-2′-{6-amino-5-[1-(1-methyl-1H-pyrazol-5-yl)ethoxy]pyridin-3-yl}-N-isopropyl-5′,6′-dihydro-1H-spiro[pyrrolidine-3,4′-pyrrolo[1,2-b]pyrazole]-1-carboxamide (Diastereomer 2)

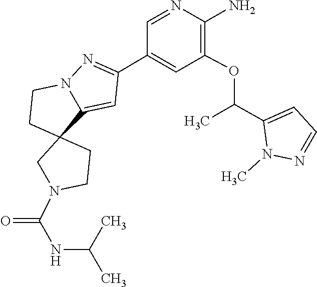 [SMILES: CC(C)NC(=O)N1CC[C@@]2(CCn3nc(-c4cnc(N)c(OC(C)c5ccnn5C)c4)cc32)C1]

For the preparation of the diastereomeric mixture of the title compound see Example 662. The diastereomers were separated by preparative chiral HPLC (method see Example 662) to give 11.4 mg (95% purity, 26% yield) of the title compound (diastereomer 2, Rₜ=7.2-7.8 min).

Analytical chiral HPLC (method see Example 662): Rₜ=3.71 min.

[α]²⁰_{D}: +0.8° (c=1.00, MeOH)

¹H-NMR (400 MHz, CHLOROFORM-d) δ [ppm]: 1.182 (4.30), 1.188 (4.94), 1.197 (5.21), 1.204 (4.04), 1.263 (1.02), 1.757 (5.65), 1.773 (5.79), 2.092 (0.63), 2.106 (0.48), 2.240 (0.68), 2.258 (0.44), 2.271 (0.46), 2.552 (0.54), 2.567 (0.62), 2.584 (0.92), 2.601 (0.47), 2.628 (0.47), 2.646 (1.02), 2.663 (0.60), 2.679 (0.59), 3.501 (0.68), 3.525 (1.75), 3.547 (0.81), 3.559 (1.79), 3.584 (0.76), 3.623 (0.40), 3.629 (0.48), 3.643 (0.41), 3.868 (16.00), 4.002 (1.39), 4.014 (0.92), 4.029 (0.58), 4.250 (1.54), 4.267 (2.98), 4.285 (1.47), 4.735 (0.82), 5.310 (4.74), 5.568 (1.05), 5.583 (1.06), 6.166 (4.58), 6.337 (2.22), 6.342 (2.21), 7.446 (2.47), 7.451 (3.26), 7.457 (1.89), 8.043 (1.51), 8.047 (1.48).

### Example 664

### (3R)-2′-{6-amino-5-[1-(1-methyl-1H-pyrazol-5-yl)ethoxy]pyridin-3-yl}-N-cyclobutyl-5′,6′-dihydro-1H-spiro[pyrrolidine-3,4′-pyrrolo[1,2-b]pyrazole]-1-carboxamide (Diastereomer 1)

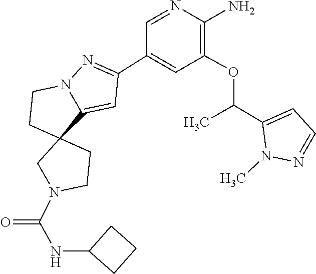 [SMILES: CC(Oc1cc(-c2cc3n(n2)CC[C@@]32CCN(C(=O)NC3CCC3)C2)cnc1N)c1ccnn1C]

5-[(3R)-5′,6′-Dihydrospiro[pyrrolidine-3,4′-pyrrolo[1,2-b]pyrazol]-2′-yl]-3-[1-(1-methyl-1H-pyrazol-5-yl)ethoxy]pyridin-2-amine-hydrochloride (Intermediate 519, 60.0 mg, 80% purity, 115 μmol) was suspended in dichloromethane (2.0 mL). N,N-Diisopropylethylamine (200 μL, 1.2 mmol) was added and cooled to 0° C. Cyclobutyl isocyanate (13 μL, 140 μmol) was added and the mixture was allowed to warm up to rt over 2 h. The reaction mixture was concentrated. The residue was purified by column chromatography (silica gel, dichloromethane/methanol (0-10%) gradient). The combined fractions were diluted with water and brine and extracted 2 times with ethyl acetate. The organic phase was dried over sodium sulfate, filtered and concentrated to give 39.8 mg (72% yield) of the title compound as a diastereomeric mixture.

The diastereomers were separated by preparative chiral HPLC to give 15.0 mg (95% purity, 36% yield) of the title compound (diastereomer 1, Rₜ=9.6-11.1 min)

Preparative Chiral HPLC Method:

Instrument: PrepCon Labomatic HPLC-2; Column: YMC Cellulose SB 5μ, 250×30; eluent A: methyl tert-butyl ether+0.1 vol % diethylamine; eluent B: ethanol+0.1 vol % diethylamine; isocratic: 80% A+20% B; flow: 40 mL/min; temperature: 25° C.; UV: 280 nm

Analytical Chiral HPLC Method:

Instrument: Thermo Fisher UltiMate 3000; Column: YMC Cellulose SB 3μ, 100×4.6; eluent A: methyl tert-butyl ether+0.1 vol % diethylamine; eluent B: ethanol; isocratic: 80% A+20% B; flow: 1.4 mL/min; temperature: 25° C.; UV: 280 nm

Analytical chiral HPLC: Rₜ=4.36 min.

[α]²⁰_{D}: +94.7° (c=1.00, MeOH)

LC-MS (Method 1): Rₜ=0.87 min; MS (ESIpos): m/z=478 [M+H]⁺

¹H-NMR (400 MHz, CHLOROFORM-d) δ [ppm]: 1.263 (1.08), 1.643 (0.94), 1.660 (1.12), 1.669 (1.19), 1.686 (1.31), 1.691 (1.15), 1.696 (0.76), 1.701 (0.92), 1.712 (1.30), 1.723 (0.56), 1.757 (5.69), 1.773 (5.61), 1.798 (0.42), 1.808 (0.49), 1.816 (0.51), 1.827 (0.57), 1.837 (0.55), 2.089 (0.55), 2.102 (0.47), 2.236 (0.63), 2.254 (0.42), 2.267 (0.43), 2.358 (0.57), 2.368 (0.70), 2.378 (0.63), 2.383 (0.64), 2.386 (0.64), 2.394 (0.49), 2.548 (0.51), 2.563 (0.58), 2.580 (0.78), 2.597 (0.44), 2.640 (0.86), 2.658 (0.60), 2.673 (0.54), 3.500 (0.55), 3.525 (1.37), 3.538 (0.50), 3.552 (2.03), 3.577 (0.81), 3.633 (0.47), 3.868 (16.00), 4.248 (1.48), 4.266 (2.64), 4.283 (1.34), 4.333 (0.59), 4.348 (1.52), 4.362 (0.58), 4.727 (1.32), 5.310 (6.60), 5.565 (1.03), 5.581 (1.00), 6.161 (4.86), 6.337 (2.11), 6.342 (2.13), 7.447 (3.89), 7.452 (3.46), 8.048 (1.28), 8.051 (1.26).

### Example 665

### (3R)-2′-{6-amino-5-[1-(1-methyl-1H-pyrazol-5-yl)ethoxy]pyridin-3-yl}-N-cyclobutyl-5′,6′-dihydro-1H-spiro[pyrrolidine-3,4′-pyrrolo[1,2-b]pyrazole]-1-carboxamide (Diastereomer 2)

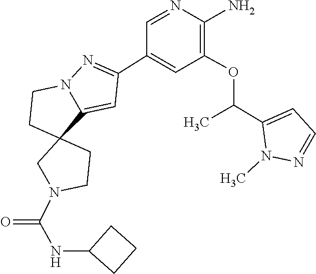 [SMILES: CC(Oc1cc(-c2cc3n(n2)CC[C@@]32CCN(C(=O)NC3CCC3)C2)cnc1N)c1ccnn1C]

For the preparation of the diastereomeric mixture of the title compound see Example 664. The diastereomers were separated by preparative chiral HPLC (method see Example 664) to give 15.6 mg (95% purity, 37% yield) of the title compound (diastereomer 2, Rₜ=11.3-13.2 min).

Analytical chiral HPLC (method see Example 664): Rₜ=5.06 min.

[α]²⁰_{D}: +6.9° (c=1.00, MeOH)

¹H-NMR (400 MHz, CHLOROFORM-d) δ [ppm]: 1.263 (1.08), 1.644 (0.65), 1.660 (0.97), 1.670 (1.12), 1.687 (1.26), 1.691 (1.10), 1.696 (0.75), 1.701 (0.88), 1.714 (1.22), 1.723 (0.55), 1.727 (0.49), 1.757 (5.25), 1.773 (5.24), 1.810 (0.45), 1.818 (0.45), 1.829 (0.52), 1.839 (0.49), 2.090 (0.50), 2.104 (0.42), 2.236 (0.55), 2.359 (0.49), 2.370 (0.64), 2.378 (0.57), 2.383 (0.57), 2.387 (0.56), 2.396 (0.43), 2.547 (0.48), 2.562 (0.53), 2.580 (0.75), 2.596 (0.40), 2.639 (0.84), 2.658 (0.52), 2.672 (0.52), 3.503 (0.50), 3.528 (1.46), 3.557 (1.71), 3.582 (0.63), 3.868 (16.00), 4.247 (1.35), 4.264 (2.52), 4.282 (1.26), 4.335 (0.51), 4.348 (1.33), 4.362 (0.53), 4.732 (1.05), 5.310 (5.94), 5.567 (0.90), 5.583 (0.89), 6.164 (4.54), 6.338 (1.91), 6.342 (1.91), 7.446 (2.37), 7.450 (3.42), 8.043 (1.49), 8.046 (1.48).

### Example 666

### (3R)-2′-{6-amino-5-[(1R)-1-(2-fluorophenyl)ethoxy]pyridin-3-yl}-N-(propan-2-yl)-5′,6′-dihydrospiro[pyrrolidine-3,4′-pyrrolo[1,2-b]pyrazole]-1-carboxamide

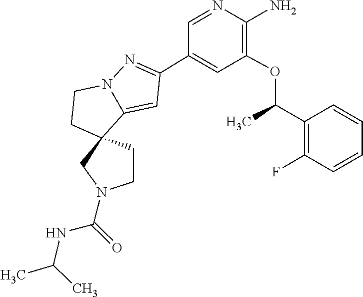 [SMILES: CC(C)NC(=O)N1CC[C@@]2(CCn3nc(-c4cnc(N)c(O[C@H](C)c5ccccc5F)c4)cc32)C1]

(3R)-1-[(Propan-2-yl)carbamoyl]-5′,6′-dihydrospiro[pyrrolidine-3,4′-pyrrolo[1,2-b]pyrazol]-2′-yl trifluoromethanesulfonate (75.0 mg, 189 μmol), {6-amino-5-[(1R)-1-(2-fluorophenyl)ethoxy]pyridin-3-yl}boronic acid (Intermediate 458, 293 mg, 25% purity, 265 μmol) and potassium phosphate (1.1 mL, 0.50 M, 570 μmol) were dissolved in degassed 1,4-dioxane (3.1 mL) under argon. XPhos Pd G2 (7.44 mg, 9.46 μmol) was added and the mixture was stirred for 2 h at 100° C. The reaction mixture was allowed to cool down, diluted with ethyl acetate and filtered. The filtrate was washed with water and brine. The organic phase was dried over sodium sulfate, filtered and concentrated. The crude product was purified by preparative TLC (gradient: dichloromethane/methanol 9:1) to give 46.0 mg (95% purity, 48% yield) of the title compound.

LC-MS (Method 1): Rₜ=1.06 min; MS (ESIpos): m/z=480 [M+H]⁺

¹H-NMR (400 MHz, CHLOROFORM-d) δ [ppm]: 1.164 (3.29), 1.172 (3.53), 1.180 (3.73), 1.187 (3.05), 1.241 (2.00), 1.684 (3.57), 1.700 (3.51), 2.054 (0.40), 2.207 (0.51), 2.525 (0.44), 2.542 (0.58), 2.599 (0.66), 2.632 (0.42), 3.484 (0.96), 3.495 (0.44), 3.512 (1.45), 3.519 (0.63), 3.537 (0.69), 3.982 (0.85), 3.996 (0.56), 4.211 (1.13), 4.229 (1.99), 4.246 (1.01), 4.778 (1.65), 5.299 (5.19), 5.754 (0.68), 5.770 (0.67), 6.063 (4.26), 7.040 (0.41), 7.058 (0.49), 7.060 (0.55), 7.063 (0.46), 7.066 (0.45), 7.084 (0.45), 7.086 (0.51), 7.113 (0.89), 7.116 (0.80), 7.132 (0.57), 7.135 (0.50), 7.242 (0.51), 7.260 (16.00), 7.267 (1.68), 7.408 (0.65), 7.412 (0.64), 8.007 (1.92), 8.011 (1.92).

### Example 667

### 2′-[6-amino-5-(trifluoromethyl)pyridin-3-yl]-N-[1-(1-methyl-1H-pyrazol-5-yl)ethyl]-5′,6′-dihydro-1H-spiro[azetidine-3,4′-pyrrolo[1,2-b]pyrazole]-1-carboxamide (Enantiomer 1 and Enantiomer 2)

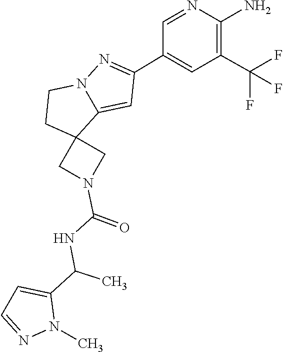 [SMILES: CC(NC(=O)N1CC2(CCn3nc(-c4cnc(N)c(C(F)(F)F)c4)cc32)C1)c1ccnn1C]

1-(1-Methyl-1H-pyrazol-5-yl)ethan-1-amine (25.0 mg, 200 μmol), CDI (35.3 mg, 218 μmol) and N,N-diisopropylethylamine (130 μL, 730 μmol) were dissolved in DMF and stirred for 1 h. A solution of 5-(5′,6′-dihydrospiro[azetidine-3,4′-pyrrolo[1,2-b]pyrazol]-2′-yl)-3-(trifluoromethyl)pyridin-2-amine-hydrogen chloride (62.8 mg, 182 μmol) in DMF was added dropwise. The mixture was stirred over night at rt. The reaction mixture was diluted with water and lyophilised. The crude product was purified by column chromatography (silica gel, dichloromethane/methanol (0-15%) gradient) to give 63.1 mg (94% purity, 71% yield) of the title compound.

LC-MS (Method 1): Rₜ=0.88 min; MS (ESIpos): m/z=461 [M+H]⁺

¹H-NMR (400 MHz, DMSO-d6) δ [ppm]: 1.137 (0.80), 1.223 (0.64), 1.242 (2.59), 1.260 (2.59), 1.390 (4.34), 1.408 (4.38), 2.332 (0.49), 2.518 (3.76), 2.522 (2.36), 2.819 (1.09), 2.838 (1.80), 2.854 (1.21), 3.159 (7.53), 3.171 (7.78), 3.670 (0.52), 3.696 (0.59), 3.720 (0.52), 3.748 (0.54), 3.771 (16.00), 3.972 (0.90), 3.993 (2.50), 4.001 (4.58), 4.010 (2.38), 4.031 (0.84), 4.086 (0.59), 4.100 (2.44), 4.118 (2.46), 4.135 (1.29), 4.948 (0.59), 4.969 (0.71), 4.986 (0.58), 6.182 (2.37), 6.186 (2.39), 6.544 (3.03), 6.717 (5.88), 6.868 (1.39), 6.889 (1.32), 7.070 (1.84), 7.289 (2.63), 7.293 (2.58), 7.759 (0.73), 8.034 (1.72), 8.040 (1.78), 8.614 (1.64), 8.619 (1.63).

### Example 668

### 2′-[6-amino-5-(trifluoromethyl)pyridin-3-yl]-N-[1-(1-methyl-1H-pyrazol-5-yl)ethyl]-5′,6′-dihydro-1H-spiro[azetidine-3,4′-pyrrolo[1,2-b]pyrazole]-1-carboxamide (Enantiomer 1)

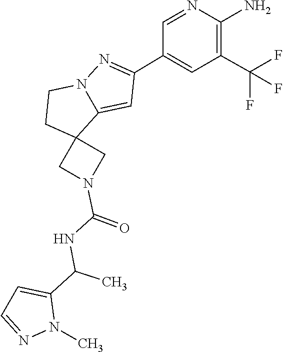 [SMILES: CC(NC(=O)N1CC2(CCn3nc(-c4cnc(N)c(C(F)(F)F)c4)cc32)C1)c1ccnn1C]

The title compound from Example 667 was separated into enantiomers by preparative chiral HPLC to give 8.20 mg (100% purity) of the title compound (enantiomer 1, Rₜ=8.4-10.4 min).

Preparative Chiral HPLC Method:

Instrument: Sepiatec Prep SFC100; Column: Chiralpak IA 5μ, 250×30; eluent A: CO2; eluent B: methanol+0.2% aqueous ammonia (32%); isocratic: 20% B; flow: 100 mL/min; temperature: 40° C.; BPR: 150 bar UV: 254 nm

Analytical Chiral HPLC Method:

Instrument: Agilent 1260, Aurora SFC-Modul; Column: Chiralpak IA 3μ, 100×4.6; eluent A: CO2; eluent B: methanol+0.2% aqueous ammonia (32%); isocratic: 20% B; flow: 4 mL/min; temperature: 37.5° C.; BPR: 100 bar; UV: 254 nm

Analytical chiral HPLC: Rₜ=2.49 min.

¹H-NMR (400 MHz, DMSO-d6) δ [ppm]: 1.390 (4.02), 1.407 (4.02), 2.518 (1.76), 2.523 (1.23), 2.819 (1.01), 2.836 (1.61), 2.854 (1.09), 2.994 (0.67), 3.720 (0.56), 3.771 (16.00), 3.972 (0.83), 3.993 (2.35), 4.001 (4.23), 4.011 (2.11), 4.032 (0.75), 4.100 (1.14), 4.118 (1.80), 4.135 (1.07), 4.948 (0.53), 4.967 (0.63), 4.986 (0.52), 6.182 (2.13), 6.186 (2.15), 6.544 (2.70), 6.717 (5.67), 6.871 (1.27), 6.892 (1.24), 7.289 (2.38), 7.294 (2.31), 8.034 (1.58), 8.040 (1.58), 8.614 (1.41), 8.618 (1.37).

### Example 669

### 2′-[6-amino-5-(trifluoromethyl)pyridin-3-yl]-N-[1-(1-methyl-1H-pyrazol-5-yl)ethyl]-5′,6′-dihydro-1H-spiro[azetidine-3,4′-pyrrolo[1,2-b]pyrazole]-1-carboxamide (Enantiomer 2)

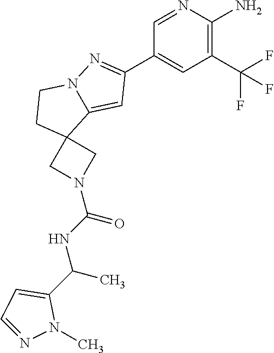 [SMILES: CC(NC(=O)N1CC2(CCn3nc(-c4cnc(N)c(C(F)(F)F)c4)cc32)C1)c1ccnn1C]

For the preparation of the racemate of the title compound see Example 667. Separation of enantiomers by preparative chiral HPLC (method see Example 668) to give 9.10 mg (100% purity) of the title compound (enantiomer 2, Rₜ=13.3-16.3 min).

Analytical chiral HPLC (method see Example 668): Rₜ=3.91 min.

¹H-NMR (400 MHz, DMSO-d6) δ [ppm]: 1.391 (3.73), 1.408 (3.77), 2.084 (0.56), 2.518 (1.80), 2.523 (1.28), 2.820 (0.94), 2.837 (1.49), 2.855 (1.03), 2.994 (0.96), 3.671 (0.43), 3.771 (16.00), 3.972 (0.78), 3.993 (2.16), 4.001 (3.88), 4.011 (1.96), 4.032 (0.72), 4.101 (1.06), 4.119 (1.69), 4.136 (1.02), 4.948 (0.50), 4.968 (0.58), 4.986 (0.48), 6.183 (2.04), 6.186 (2.07), 6.546 (2.50), 6.718 (5.36), 6.868 (1.20), 6.890 (1.15), 7.289 (2.24), 7.294 (2.21), 8.035 (1.47), 8.040 (1.48), 8.615 (1.36), 8.619 (1.34).

### Example 670

### 2′-[6-amino-5-(trifluoromethyl)pyridin-3-yl]-N-[1-(1,3-dimethyl-1H-pyrazol-4-yl)ethyl]-5′,6′-dihydro-1H-spiro[azetidine-3,4′-pyrrolo[1,2-b]pyrazole]-1-carboxamide (Enantiomer 1 and Enantiomer 2)

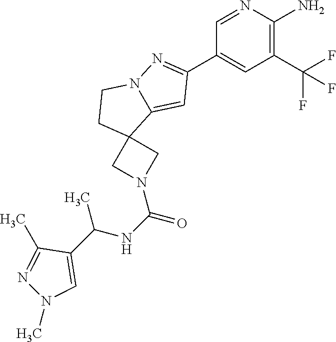 [SMILES: Cc1nn(C)cc1C(C)NC(=O)N1CC2(CCn3nc(-c4cnc(N)c(C(F)(F)F)c4)cc32)C1]

1-(1,3-Dimethyl-1H-pyrazol-4-yl)ethan-1-amine (33.2 mg, 239 μmol), CDI (42.2 mg, 260 μmol) and N,N-diisopropylethylamine (150 μL, 870 μmol) were dissolved in DMF and stirred for 1 h. A solution of 5-(5′,6′-dihydrospiro[azetidine-3,4′-pyrrolo[1,2-b]pyrazol]-2′-yl)-3-(trifluoromethyl)pyridin-2-amine-hydrogen chloride (75.0 mg, 217 μmol) in DMF was added dropwise. The mixture was stirred over night at rt. The reaction mixture was diluted with water and lyophilised. The crude product was purified by column chromatography (silica gel, dichloromethane/methanol (0-15%) gradient) to give 112 mg (90% purity, 98% yield) of the title compound.

LC-MS (Method 1): Rₜ=0.87 min; MS (ESIpos): m/z=475 [M+H]⁺

¹H-NMR (400 MHz, DMSO-d6) δ [ppm]: 1.237 (1.93), 1.241 (1.37), 1.256 (11.78), 1.273 (9.37), 1.307 (1.86), 1.324 (1.82), 2.039 (1.61), 2.067 (1.68), 2.089 (5.25), 2.518 (1.16), 2.523 (0.74), 2.810 (0.49), 2.827 (0.82), 2.845 (0.53), 3.098 (0.93), 3.117 (0.92), 3.164 (16.00), 3.336 (0.71), 3.571 (0.65), 3.604 (0.63), 3.674 (1.72), 3.682 (1.77), 3.695 (5.28), 3.941 (0.44), 3.962 (0.99), 3.972 (1.32), 3.975 (1.40), 3.983 (1.02), 4.003 (0.46), 4.100 (0.69), 4.117 (1.01), 4.135 (0.67), 6.534 (0.87), 6.542 (1.37), 6.554 (0.73), 6.703 (2.30), 7.052 (3.98), 7.055 (4.04), 7.411 (0.49), 7.416 (0.49), 7.449 (1.51), 7.728 (1.56), 8.035 (0.74), 8.040 (0.76), 8.615 (0.71), 8.619 (0.71).

### Example 671

### 2′-[6-amino-5-(trifluoromethyl)pyridin-3-yl]-N-[1-(1,3-dimethyl-1H-pyrazol-4-yl)ethyl]-5′,6′-dihydro-1H-spiro[azetidine-3,4′-pyrrolo[1,2-b]pyrazole]-1-carboxamide (Enantiomer 1)

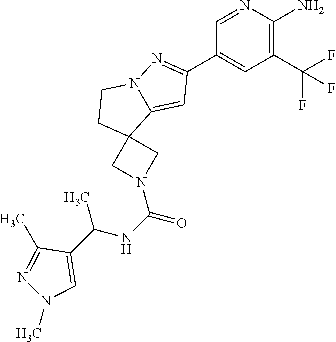 [SMILES: Cc1nn(C)cc1C(C)NC(=O)N1CC2(CCn3nc(-c4cnc(N)c(C(F)(F)F)c4)cc32)C1]

The title compound from Example 670 was separated into enantiomers by preparative chiral HPLC to give 24.6 mg (100% purity) of the title compound (enantiomer 1, Rₜ=8.0-10.0 min)

Preparative Chiral HPLC Method:

Instrument: Sepiatec Prep SFC100; Column: Reprosil Chiral NR 8μ, 250×30; eluent A: CO2; eluent B: methanol+0.2% aqueous ammonia (32%); isocratic: 30% B; flow: 100 mL/min; temperature: 40° C.; BPR: 150 bar UV: 254 nm

Analytical Chiral HPLC Method:

Instrument: Agilent 1260, Aurora SFC-Modul; Column: Reprosil Chiral NR 5μ, 100×4.6; eluent A: CO2; eluent B: methanol+0.2% aqueous ammonia (32%); isocratic: 30% B; flow: 4 mL/min; temperature: 37.5° C.; BPR: 100 bar; UV: 254 nm

Analytical chiral HPLC: Rₜ=2.67 min.

¹H-NMR (400 MHz, DMSO-d6) δ [ppm]: 1.250 (0.75), 1.269 (2.12), 1.287 (1.42), 1.307 (5.14), 1.324 (5.05), 2.066 (0.48), 2.088 (16.00), 2.366 (0.68), 2.518 (2.59), 2.523 (2.15), 2.709 (0.72), 2.810 (1.49), 2.827 (2.34), 2.844 (1.56), 2.994 (2.23), 3.109 (0.62), 3.127 (0.63), 3.146 (0.41), 3.385 (0.58), 3.519 (0.57), 3.563 (0.53), 3.580 (0.65), 3.597 (0.70), 3.613 (0.61), 3.629 (0.47), 3.681 (0.91), 3.941 (1.39), 3.951 (0.78), 3.961 (2.90), 3.971 (3.79), 3.975 (3.95), 3.982 (2.85), 3.996 (0.76), 4.003 (1.24), 4.099 (1.60), 4.117 (2.43), 4.134 (1.52), 4.686 (0.76), 4.706 (0.90), 4.724 (0.74), 6.543 (3.92), 6.559 (1.88), 6.702 (7.34), 7.450 (4.40), 7.556 (4.69), 8.035 (2.13), 8.040 (2.14), 8.614 (1.98), 8.618 (1.95), 8.804 (2.56).

### Example 672

2′-[6-amino-5-(trifluoromethyl)pyridin-3-yl]-N-[1-(1,3-dimethyl-1H-pyrazol-4-yl)ethyl]-5′,6′-dihydro-1H-spiro[azetidine-3,4′-pyrrolo[1,2-b]pyrazole]-1-carboxamide (Enantiomer 2)

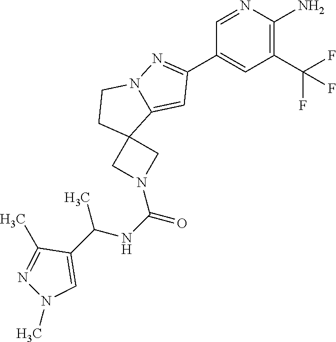 [SMILES: Cc1nn(C)cc1C(C)NC(=O)N1CC2(CCn3nc(-c4cnc(N)c(C(F)(F)F)c4)cc32)C1]

For the preparation of the racemate of the title compound see Example 670. The enantiomers were separated by preparative chiral HPLC (method see Example 671) to give 22.2 mg (100% purity) of the title compound (enantiomer 2, Rₜ=11.1-14.1 min).

Analytical chiral HPLC (method see Example 671): Rₜ=3.69 min.

¹H-NMR (400 MHz, DMSO-d6) δ [ppm]: 1.244 (1.28), 1.261 (1.31), 1.308 (5.12), 1.325 (5.12), 2.067 (3.38), 2.084 (3.88), 2.090 (16.00), 2.518 (1.54), 2.523 (1.10), 2.810 (1.38), 2.828 (2.24), 2.845 (1.46), 3.683 (3.53), 3.696 (15.65), 3.942 (1.24), 3.951 (0.64), 3.963 (2.78), 3.971 (3.60), 3.975 (3.79), 3.983 (2.78), 3.996 (0.65), 4.004 (1.11), 4.100 (1.54), 4.118 (2.35), 4.135 (1.47), 4.688 (0.74), 4.708 (0.89), 4.726 (0.72), 6.528 (1.96), 6.546 (4.08), 6.704 (7.65), 7.415 (0.89), 7.447 (4.42), 8.036 (2.14), 8.041 (2.18), 8.616 (1.92), 8.620 (1.91).

### Example 673

### (3S)-2′-[6-amino-5-(trifluoromethyl)pyridin-3-yl]-N-[1-(1-methyl-1H-pyrazol-5-yl)ethyl]-5′,6′-dihydro-1H-spiro[pyrrolidine-3,4′-pyrrolo[1,2-b]pyrazole]-1-carboxamide (Diastereomer 1 and Diastereomer 2)

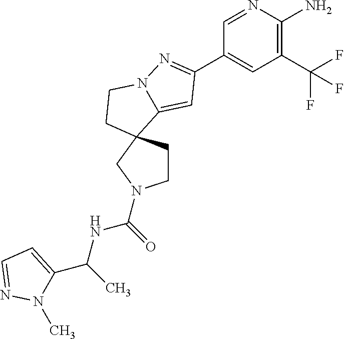 [SMILES: CC(NC(=O)N1CC[C@]2(CCn3nc(-c4cnc(N)c(C(F)(F)F)c4)cc32)C1)c1ccnn1C]

1-(1-Methyl-1H-pyrazol-5-yl)ethan-1-amine (25.0 mg, 200 μmol), CDI (35.3 mg, 218 μmol) and N,N-diisopropylethylamine (130 μL, 730 μmol) were dissolved in DMF and stirred for 1 h. A solution of 5-[(3S)-5′,6′-dihydrospiro[pyrrolidine-3,4′-pyrrolo[1,2-b]pyrazol]-2′-yl]-3-(trifluoromethyl)pyridin-2-amine-hydrogen chloride (65.3 mg, 182 μmol) in DMF was added dropwise. The mixture was stirred over night at rt and concentrated. The residue was purified by preparative HPLC to give 25.0 mg (94% purity, 27% yield) of the title compound.

LC-MS (Method 1): Rₜ=0.90 min; MS (ESIpos): m/z=475 [M+H]⁺

¹H-NMR (400 MHz, DMSO-d6) δ [ppm]: 1.137 (0.54), 1.388 (4.64), 1.397 (5.02), 1.405 (4.99), 1.415 (4.59), 1.751 (0.86), 2.057 (2.35), 2.084 (3.58), 2.115 (0.41), 2.323 (0.87), 2.327 (1.18), 2.331 (0.88), 2.518 (8.85), 2.523 (7.23), 2.665 (0.88), 2.669 (1.20), 2.673 (0.88), 3.397 (1.65), 3.423 (4.12), 3.436 (3.00), 3.442 (3.06), 3.450 (1.25), 3.468 (1.24), 3.488 (0.42), 3.507 (0.78), 3.521 (0.70), 3.534 (0.95), 3.550 (0.61), 3.561 (0.52), 3.751 (15.77), 3.764 (16.00), 4.158 (2.52), 4.175 (4.69), 4.193 (2.41), 4.986 (1.19), 5.005 (1.58), 5.023 (1.20), 5.758 (4.29), 6.157 (2.23), 6.162 (2.29), 6.185 (2.26), 6.190 (2.32), 6.442 (3.40), 6.447 (5.96), 6.454 (1.55), 6.468 (1.49), 6.475 (1.47), 6.489 (1.30), 6.546 (6.54), 7.252 (2.42), 7.256 (2.44), 7.268 (2.60), 7.273 (2.57), 8.002 (3.55), 8.006 (3.58), 8.576 (3.52), 8.580 (3.47).

### Example 674

### (3S)-2′-[6-amino-5-(trifluoromethyl)pyridin-3-yl]-N-[1-(1,3-dimethyl-1H-pyrazol-4-yl)ethyl]-5′,6′-dihydro-1H-spiro[pyrrolidine-3,4′-pyrrolo[1,2-b]pyrazole]-1-carboxamide (Diastereomer 1 and Diastereomer 2)

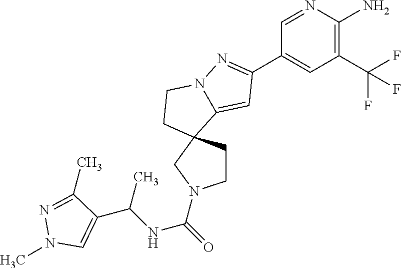 [SMILES: Cc1nn(C)cc1C(C)NC(=O)N1CC[C@]2(CCn3nc(-c4cnc(N)c(C(F)(F)F)c4)cc32)C1]

1-(1,3-Dimethyl-1H-pyrazol-4-yl)ethan-1-amine (31.9 mg, 229 μmol), CDI (40.6 mg, 250 μmol) and N,N-diisopropylethylamine (150 μL, 830 μmol) were dissolved in DMF and stirred for 1 h. A solution of 5-[(3S)-5′,6′-dihydrospiro[pyrrolidine-3,4′-pyrrolo[1,2-b]pyrazol]-2′-yl]-3-(trifluoromethyl)pyridin-2-amine-hydrogen chloride (75.0 mg, 208 μmol) in DMF was added dropwise. The mixture was stirred over night at rt and concentrated. The residue was purified by preparative HPLC to give 18.8 mg (96% purity, 18% yield) of the title compound.

LC-MS (Method 1): Rₜ=0.89 min; MS (ESIpos): m/z=489 [M+H]⁺

¹H-NMR (400 MHz, DMSO-d6) δ [ppm]: 1.137 (0.69), 1.305 (2.69), 1.315 (2.91), 1.322 (2.97), 1.332 (2.60), 1.750 (0.52), 2.048 (1.41), 2.062 (8.20), 2.083 (8.34), 2.331 (1.17), 2.336 (0.53), 2.518 (8.71), 2.523 (5.83), 2.673 (1.15), 2.678 (0.53), 3.369 (0.98), 3.373 (0.92), 3.394 (1.76), 3.409 (0.58), 3.416 (0.44), 3.430 (1.98), 3.455 (0.99), 3.482 (0.43), 3.506 (0.40), 3.523 (0.47), 3.672 (7.53), 3.689 (7.86), 4.158 (1.38), 4.175 (2.72), 4.193 (1.34), 4.734 (0.54), 4.751 (0.81), 4.767 (0.53), 5.759 (16.00), 6.083 (0.83), 6.090 (0.83), 6.104 (0.83), 6.110 (0.78), 6.442 (2.88), 6.444 (4.28), 6.544 (3.48), 7.427 (2.05), 7.454 (2.16), 8.003 (1.87), 8.577 (1.96).

### Example 675

### (3R)-2′-{6-amino-5-[(1R)-1-(2,6-difluorophenyl)ethoxy]pyridin-3-yl}-N-(propan-2-yl)-5′,6′-dihydrospiro[pyrrolidine-3,4′-pyrrolo[1,2-b]pyrazole]-1-carboxamide

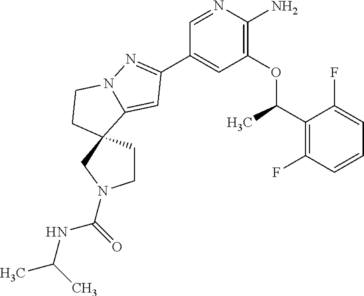 [SMILES: CC(C)NC(=O)N1CC[C@@]2(CCn3nc(-c4cnc(N)c(O[C@H](C)c5c(F)cccc5F)c4)cc32)C1]

(3R)-1-[(Propan-2-yl)carbamoyl]-5′,6′-dihydrospiro[pyrrolidine-3,4′-pyrrolo[1,2-b]pyrazol]-2′-yl trifluoromethanesulfonate (47.4 mg, 120 μmol), {6-amino-5-[(1R)-1-(2,6-difluorophenyl)ethoxy]pyridin-3-yl}boronic acid (Intermediate 447, 154 mg, 32% purity, 168 μmol) and potassium phosphate (720 μL, 0.50 M, 360 μmol) were dissolved in degassed 1,4-dioxane (2.0 mL) under argon. XPhos Pd G2 (4.71 mg, 5.99 μmol) was added and the mixture was stirred for 2 h at 100° C. XPhos Pd G2 (10 mg, 12.7 μmol) was added again and stirred for 3 h at 100° C. The reaction mixture was concentrated. The residue was filtered over silica gel with dichloromethane/methanol 80:20. The filtrate was concentrated.

The residue was dissolved in 1,4-dioxane. XPhos Pd G2 (4.71 mg, 5.99 μmol), potassium phosphate (720 μL, 0.50 M, 360 μmol) and (3R)-1-[(propan-2-yl)carbamoyl]-5′,6′-dihydrospiro[pyrrolidine-3,4′-pyrrolo[1,2-b]pyrazol]-2′-yl trifluoromethanesulfonate (20 mg, 50.6 μmol) were added and the mixture was stirred for 2 h at 100° C. The reaction mixture was allowed to cool down, diluted with ethy acetate and filtered. The filtrate was washed with water and brine, dried over sodium sulfate, filtered and concentrated. The crude product was purified by preparative TLC (gradient: dichloromethane/methanol 9:1) to give 10.5 mg (95% purity, 17% yield) of the title compound.

LC-MS (Method 1): Rₜ=1.01 min; MS (ESIpos): m/z=498 [M+H]⁺

¹H-NMR (400 MHz, CHLOROFORM-d) δ [ppm]: 0.888 (0.47), 1.181 (14.12), 1.188 (15.39), 1.197 (16.00), 1.203 (12.91), 1.251 (5.07), 1.261 (1.95), 1.801 (13.12), 1.818 (12.98), 2.050 (0.72), 2.063 (0.96), 2.068 (0.98), 2.081 (1.80), 2.095 (1.59), 2.112 (1.02), 2.216 (1.03), 2.235 (2.10), 2.247 (1.00), 2.254 (1.28), 2.266 (1.48), 2.285 (0.81), 2.520 (0.76), 2.537 (1.67), 2.553 (1.83), 2.570 (2.92), 2.587 (1.46), 2.630 (3.20), 2.648 (1.80), 2.663 (1.68), 2.681 (0.75), 3.489 (1.84), 3.501 (1.02), 3.513 (4.31), 3.525 (1.61), 3.546 (5.99), 3.561 (1.20), 3.571 (2.35), 3.609 (0.98), 3.622 (1.15), 3.629 (1.37), 3.642 (1.21), 3.653 (0.79), 3.666 (0.59), 3.981 (1.09), 4.000 (4.27), 4.015 (2.43), 4.030 (1.71), 4.048 (0.84), 4.245 (4.71), 4.263 (9.28), 4.281 (4.52), 4.806 (5.82), 5.310 (6.09), 5.868 (0.86), 5.884 (2.73), 5.901 (2.68), 5.917 (0.84), 6.112 (15.20), 6.855 (0.58), 6.865 (3.94), 6.877 (0.89), 6.885 (8.38), 6.906 (4.48), 6.915 (0.70), 7.006 (0.74), 7.198 (0.88), 7.214 (1.86), 7.219 (1.50), 7.230 (1.20), 7.235 (3.17), 7.240 (1.08), 7.250 (1.66), 7.256 (1.85), 7.429 (5.47), 7.433 (5.43), 7.528 (0.72), 8.025 (6.98), 8.029 (6.97).

### Example 676

### (3R)-2′-(6-amino-5-{methyl[(1R)-1-phenylethyl]amino}pyridin-3-yl)-N-ethyl-5′,6′-dihydrospiro[pyrrolidine-3,4′-pyrrolo[1,2-b]pyrazole]-1-carboxamide

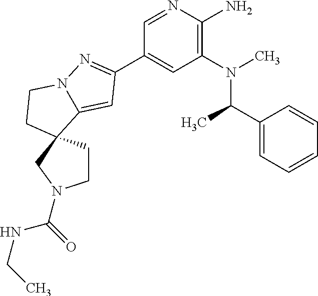 [SMILES: CCNC(=O)N1CC[C@@]2(CCn3nc(-c4cnc(N)c(N(C)[C@H](C)c5ccccc5)c4)cc32)C1]

(3R)-1-(Ethylcarbamoyl)-5′,6′-dihydrospiro[pyrrolidine-3,4′-pyrrolo[1,2-b]pyrazol]-2′-yl trifluoromethanesulfonate (44.2 mg, 116 μmol), (6-amino-5-{methyl[(1R)-1-phenylethyl]amino}pyridin-3-yl)boronic acid (Intermediate 459, 258 mg, 17% purity, 162 μmol) and potassium phosphate (690 μL, 0.50 M, 350 μmol) were dissolved in degassed 1,4-dioxane (1.9 mL) under argon. XPhos Pd G2 (4.55 mg, 5.78 μmol) was added and the mixture was stirred for 2 h at 100° C. The reaction mixture was allowed to cool down, diluted with ethy acetate and filtered. The filtrate was washed with water and brine, dried over sodium sulfate, filtered and concentrated. The residue was purified by flash column chromatography (silica gel, dichloromethane/methanol (0-20%) gradient) to give 37.0 mg (98% purity, 68% yield) of the title compound.

LC-MS (Method 1): Rₜ=1.05 min; MS (ESIpos): m/z=461 [M+H]⁺

¹H-NMR (400 MHz, CHLOROFORM-d) δ [ppm]: 1.164 (5.54), 1.182 (12.13), 1.200 (5.69), 1.341 (7.63), 1.358 (7.66), 2.082 (0.52), 2.096 (0.89), 2.109 (0.77), 2.127 (0.53), 2.222 (0.54), 2.241 (1.01), 2.253 (0.54), 2.258 (0.69), 2.270 (0.69), 2.289 (0.40), 2.480 (16.00), 2.551 (0.89), 2.566 (1.01), 2.584 (1.38), 2.600 (0.77), 2.626 (0.82), 2.643 (1.57), 2.662 (0.96), 2.676 (0.95), 2.694 (0.42), 3.295 (0.76), 3.308 (1.01), 3.312 (2.49), 3.326 (2.61), 3.330 (2.48), 3.344 (2.50), 3.349 (0.94), 3.363 (0.74), 3.510 (1.01), 3.534 (2.25), 3.545 (0.87), 3.552 (0.75), 3.570 (3.78), 3.587 (0.66), 3.594 (1.32), 3.626 (0.53), 3.640 (0.67), 3.646 (0.77), 3.659 (0.64), 3.665 (0.53), 3.670 (0.44), 4.165 (0.62), 4.178 (1.11), 4.191 (0.59), 4.259 (2.52), 4.277 (4.55), 4.294 (2.47), 4.305 (1.68), 4.321 (1.57), 4.338 (0.46), 5.003 (3.58), 5.310 (14.30), 6.175 (8.80), 7.245 (0.40), 7.250 (0.67), 7.255 (0.53), 7.278 (0.93), 7.284 (1.39), 7.288 (0.87), 7.322 (1.29), 7.338 (1.22), 7.343 (3.83), 7.360 (7.49), 7.364 (5.01), 7.381 (1.18), 7.386 (0.75), 7.655 (3.20), 7.660 (3.21), 8.222 (3.97), 8.227 (3.86).

### Example 677

### 2′-[6-amino-5-(trifluoromethyl)pyridin-3-yl]-N-[1-(1,3,5-trimethyl-1H-pyrazol-4-yl)ethyl]-5′,6′-dihydro-1H-spiro[azetidine-3,4′-pyrrolo[1,2-b]pyrazole]-1-carboxamide (Diastereomer 1 and Diastereomer 2)

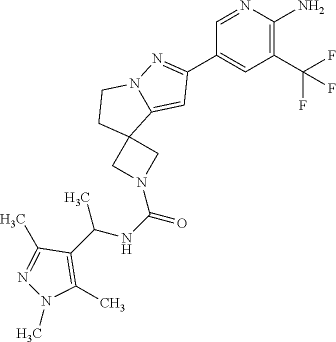 [SMILES: Cc1nn(C)c(C)c1C(C)NC(=O)N1CC2(CCn3nc(-c4cnc(N)c(C(F)(F)F)c4)cc32)C1]

1-(1,3,5-Trimethyl-1H-pyrazol-4-yl)ethan-1-amine (36.6 mg, 239 μmol), CDI (42.2 mg, 260 μmol) and N,N-diisopropylethylamine (150 μL, 870 μmol) were dissolved in DMF and stirred for 1 h at rt. A solution of 5-(5′,6′-dihydrospiro[azetidine-3,4′-pyrrolo[1,2-b]pyrazol]-2′-yl)-3-(trifluoromethyl)pyridin-2-amine-hydrogen chloride (75.0 mg, 217 μmol) in DMF was added dropwise. The mixture was stirred over night at rt. The reaction mixture was diluted with water and lyophilised. The residue was purified by flash column chromatography (silica gel, dichloromethane/methanol (0-15%) gradient) to give 101 mg (95% purity, 91% yield) of the title compound.

LC-MS (Method 1): Rₜ=0.90 min; MS (ESIpos): m/z=489 [M+H]⁺

¹H-NMR (400 MHz, DMSO-d6) δ [ppm]: 1.066 (1.93), 1.216 (0.44), 1.226 (1.15), 1.234 (0.89), 1.245 (3.78), 1.249 (4.94), 1.265 (5.04), 1.328 (4.66), 1.346 (4.76), 2.071 (0.93), 2.095 (0.87), 2.126 (13.70), 2.153 (0.96), 2.198 (14.30), 2.518 (4.47), 2.523 (3.07), 2.801 (1.17), 2.819 (1.90), 2.836 (1.26), 3.119 (0.83), 3.138 (0.90), 3.165 (16.00), 3.233 (1.06), 3.549 (1.42), 3.573 (13.30), 3.589 (0.70), 3.606 (0.73), 3.622 (0.56), 3.930 (1.12), 3.941 (0.81), 3.950 (2.15), 3.961 (2.36), 3.970 (2.62), 3.976 (2.33), 3.991 (0.91), 3.996 (1.15), 4.097 (1.57), 4.114 (2.30), 4.131 (1.50), 4.643 (0.85), 4.660 (1.32), 4.678 (0.85), 6.540 (3.03), 6.574 (1.43), 6.592 (1.44), 6.688 (6.25), 7.121 (8.76), 7.122 (9.02), 7.870 (3.28), 8.029 (1.76), 8.035 (1.81), 8.608 (1.68), 8.613 (1.66).

### Example 678

### 2′-[6-amino-5-(trifluoromethyl)pyridin-3-yl]-N-[1-(1,3,5-trimethyl-1H-pyrazol-4-yl)ethyl]-5′,6′-dihydro-1H-spiro[azetidine-3,4′-pyrrolo[1,2-b]pyrazole]-1-carboxamide (Diastereomer 1)

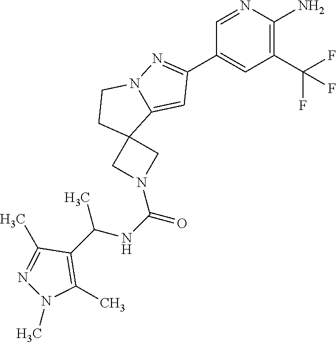 [SMILES: Cc1nn(C)c(C)c1C(C)NC(=O)N1CC2(CCn3nc(-c4cnc(N)c(C(F)(F)F)c4)cc32)C1]

The title compound from Example 677 was separated into diastereomers by preparative chiral HPLC to give 35.0 mg (96% purity) of the title compound (diastereomer 1, Rₜ=6.5-9.1 min)

Preparative Chiral HPLC Method:

Instrument: PrepCon Labomatic HPLC-2; Column: YMC Amylose SA 5μ, 250×30; eluent A: hexane+0.1 vol % diethylamine; eluent B: ethanol+0.1 vol % diethylamine; isocratic: 70% A+30% B; flow: 50 mL/min; temperature: 25° C.; UV: 254 nm

Analytical Chiral HPLC Method:

Instrument: Thermo Fisher UltiMate 3000; Column: YMC Amylose SA 3μ, 100×4.6; eluent A: hexane+0.1 vol % diethylamine; eluent B: ethanol; isocratic: 70% A+30% B; flow: 1.4 mL/min; temperature: 35° C.; UV: 254 nm

Analytical chiral HPLC: Rₜ=2.67 min.

LC-MS (Method 1): Rₜ=0.90 min; MS (ESIpos): m/z=489 [M+H]⁺

¹H-NMR (400 MHz, DMSO-d6) δ [ppm]: 0.795 (0.60), 1.006 (0.48), 1.034 (0.51), 1.066 (0.96), 1.084 (1.17), 1.259 (1.87), 1.328 (5.52), 1.346 (5.62), 2.074 (0.46), 2.127 (15.22), 2.144 (0.47), 2.198 (16.00), 2.518 (3.49), 2.523 (2.40), 2.802 (1.37), 2.819 (2.21), 2.837 (1.45), 3.574 (14.85), 3.930 (1.30), 3.941 (1.00), 3.950 (2.52), 3.961 (2.72), 3.971 (3.09), 3.976 (2.69), 3.991 (0.95), 3.997 (1.19), 4.097 (1.50), 4.115 (2.33), 4.132 (1.47), 4.643 (1.00), 4.660 (1.56), 4.678 (0.99), 6.543 (3.63), 6.574 (1.70), 6.592 (1.60), 6.690 (7.55), 8.030 (2.10), 8.035 (2.13), 8.609 (1.97), 8.613 (1.92), 10.852 (0.77).

### Example 679

### 2′-[6-amino-5-(trifluoromethyl)pyridin-3-yl]-N-[1-(1,3,5-trimethyl-1H-pyrazol-4-yl)ethyl]-5′,6′-dihydro-1H-spiro[azetidine-3,4′-pyrrolo[1,2-b]pyrazole]-1-carboxamide (Diastereomer 2)

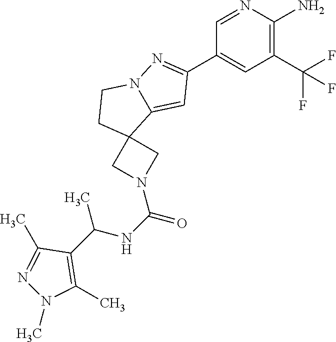 [SMILES: Cc1nn(C)c(C)c1C(C)NC(=O)N1CC2(CCn3nc(-c4cnc(N)c(C(F)(F)F)c4)cc32)C1]

For the preparation of the diastereomeric mixture of the title compound see Example 677. The diastereomers were separated by preparative chiral HPLC (method see Example 678) to give 38.0 mg (100% purity) of the title compound (diastereomer 2, Rₜ=12.3-17.3 min). Analytical chiral HPLC (method see Example 678): Rₜ=5.63 min.

LC-MS (Method 1): Rₜ=0.90 min; MS (ESIpos): m/z=489 [M+H]⁺

¹H-NMR (400 MHz, DMSO-d6) δ [ppm]: 0.795 (0.74), 0.798 (0.62), 0.814 (0.76), 0.819 (0.41), 0.821 (0.65), 0.862 (0.42), 0.904 (0.72), 1.006 (0.58), 1.034 (0.62), 1.084 (1.37), 1.259 (2.02), 1.328 (5.48), 1.346 (5.51), 2.127 (15.19), 2.199 (16.00), 2.518 (2.77), 2.523 (1.90), 2.802 (1.34), 2.819 (2.17), 2.837 (1.43), 3.574 (14.71), 3.930 (1.27), 3.941 (0.89), 3.950 (2.46), 3.961 (2.66), 3.971 (3.04), 3.976 (2.63), 3.991 (0.93), 3.997 (1.17), 4.097 (1.47), 4.115 (2.26), 4.132 (1.41), 4.643 (0.99), 4.660 (1.53), 4.678 (0.97), 6.543 (3.48), 6.574 (1.67), 6.592 (1.58), 6.690 (7.69), 8.030 (2.06), 8.035 (2.09), 8.609 (1.91), 8.613 (1.89), 10.852 (0.84).

### Example 680

### (3R)-2′-{6-amino-5-[(1R)-1-(4-methylpyridin-2-yl)ethoxy]pyridin-3-yl}-N-(propan-2-yl)-5′,6′-dihydrospiro[pyrrolidine-3,4′-pyrrolo[1,2-b]pyrazole]-1-carboxamide

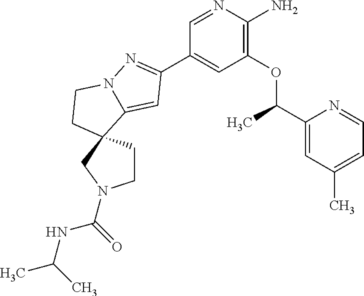 [SMILES: Cc1ccnc([C@@H](C)Oc2cc(-c3cc4n(n3)CC[C@@]43CCN(C(=O)NC(C)C)C3)cnc2N)c1]

(3R)-1-[(Propan-2-yl)carbamoyl]-5′,6′-dihydrospiro[pyrrolidine-3,4′-pyrrolo[1,2-b]pyrazol]-2′-yl trifluoromethanesulfonate (65.4 mg, 165 μmol), {6-amino-5-[(1R)-1-(4-methylpyridin-2-yl)ethoxy]pyridin-3-yl}boronic acid (Intermediate 460, 186 mg, 34% purity, 231 μmol) and potassium phosphate (990 μL, 0.50 M, 500 μmol) were dissolved in degassed 1,4-dioxane (2.7 mL) under argon. XPhos Pd G2 (6.50 mg, 8.26 μmol) was added and the mixture was stirred for 2 h at 100° C. The reaction mixture was allowed to cool down, diluted with ethy acetate and filtered. The filtrate was washed with water and brine, dried over sodium sulfate, filtered and concentrated. The residue was purified by flash column chromatography (silica gel, dichloromethane/methanol (0-20%) gradient) followed by preparative TLC (gradient: dichloromethane/methanol 9:1) to give 28.0 mg (95% purity, 34% yield) of the title compound.

LC-MS (Method 1): Rₜ=0.93 min; MS (ESIpos): m/z=477 [M+H]⁺

¹H-NMR (400 MHz, CHLOROFORM-d) δ [ppm]: 1.249 (7.43), 1.257 (8.83), 1.265 (9.73), 1.272 (7.10), 1.781 (8.80), 1.798 (8.90), 1.993 (0.72), 2.095 (0.55), 2.112 (0.72), 2.125 (1.18), 2.138 (0.97), 2.156 (0.70), 2.253 (0.67), 2.272 (1.38), 2.284 (0.68), 2.291 (0.85), 2.303 (1.00), 2.322 (0.51), 2.395 (16.00), 2.581 (0.84), 2.596 (1.09), 2.631 (0.85), 2.654 (0.86), 2.672 (1.56), 2.689 (1.13), 2.704 (0.94), 2.722 (0.45), 3.535 (0.90), 3.560 (2.75), 3.570 (1.61), 3.582 (3.93), 3.594 (1.56), 3.606 (1.41), 3.660 (0.61), 3.673 (0.78), 3.680 (0.93), 3.693 (0.82), 3.704 (0.54), 4.067 (2.49), 4.077 (2.02), 4.092 (1.05), 4.110 (0.51), 4.276 (2.62), 4.293 (4.73), 4.310 (2.49), 4.960 (4.79), 5.382 (5.54), 5.537 (0.57), 5.553 (1.85), 5.569 (1.83), 5.585 (0.55), 6.119 (0.94), 6.131 (7.38), 7.083 (1.95), 7.095 (1.92), 7.270 (3.38), 7.334 (0.56), 8.101 (3.50), 8.105 (3.63), 8.116 (0.54), 8.121 (0.47), 8.509 (2.63), 8.521 (2.58).

### Example 681

### (3R)-2′-{6-amino-5-[(1R)-1-(4-methylpyridin-2-yl)ethoxy]pyridin-3-yl}-N-cyclobutyl-5′,6′-dihydrospiro[pyrrolidine-3,4′-pyrrolo[1,2-b]pyrazole]-1-carboxamide

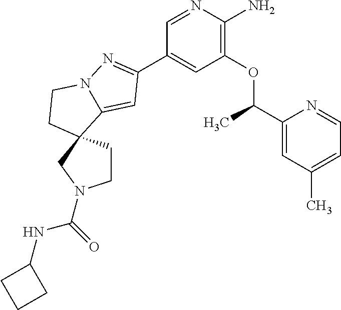 [SMILES: Cc1ccnc([C@@H](C)Oc2cc(-c3cc4n(n3)CC[C@@]43CCN(C(=O)NC4CCC4)C3)cnc2N)c1]

(3R)-1-(Cyclobutylcarbamoyl)-5′,6′-dihydrospiro[pyrrolidine-3,4′-pyrrolo[1,2-b]pyrazol]-2′-yl trifluoromethanesulfonate (67.4 mg, 165 μmol), {6-amino-5-[(1R)-1-(4-methylpyridin-2-yl)ethoxy]pyridin-3-yl}boronic acid (Intermediate 460, 186 mg, 34% purity, 231 μmol) and potassium phosphate (990 μL, 0.50 M, 500 μmol) were dissolved in degassed 1,4-dioxane (2.7 mL) under argon. XPhos Pd G2 (6.50 mg, 8.26 μmol) was added and the mixture was stirred for 2 h at 100° C. The reaction mixture was allowed to cool down, diluted with ethyl acetate and filtered. The filtrate was washed with water and brine, dried over sodium sulfate, filtered and concentrated. The residue was purified by flash column chromatography (silica gel, dichloromethane/methanol (0-20%) gradient) followed by preparative TLC (gradient: dichloromethane/methanol 9:1) to give 37.0 mg (95% purity, 44% yield) of the title compound.

LC-MS (Method 1): Rₜ=0.96 min; MS (ESIpos): m/z=489 [M+H]⁺

¹H-NMR (400 MHz, CHLOROFORM-d) δ [ppm]: 1.728 (0.75), 1.737 (1.09), 1.742 (0.61), 1.754 (1.83), 1.760 (1.50), 1.770 (1.40), 1.782 (10.80), 1.799 (9.48), 1.873 (0.77), 1.884 (0.93), 1.896 (1.03), 1.901 (1.15), 1.907 (1.13), 1.914 (1.08), 1.918 (1.00), 1.928 (0.92), 1.940 (0.75), 1.957 (0.49), 2.092 (0.42), 2.106 (0.56), 2.111 (0.58), 2.123 (1.03), 2.137 (0.83), 2.141 (0.76), 2.155 (0.61), 2.249 (0.59), 2.268 (1.25), 2.280 (0.64), 2.287 (0.76), 2.299 (0.90), 2.318 (0.46), 2.395 (16.00), 2.410 (0.59), 2.427 (1.12), 2.438 (1.40), 2.447 (1.24), 2.453 (1.22), 2.456 (1.23), 2.465 (0.99), 2.576 (0.79), 2.591 (1.05), 2.625 (0.83), 2.648 (0.84), 2.665 (1.43), 2.683 (1.07), 2.698 (0.93), 2.716 (0.45), 3.539 (0.74), 3.563 (2.41), 3.582 (4.16), 3.594 (0.89), 3.600 (1.36), 3.607 (1.25), 3.618 (0.60), 3.661 (0.56), 3.675 (0.71), 3.681 (0.83), 3.694 (0.73), 3.705 (0.47), 4.273 (2.47), 4.291 (4.43), 4.308 (2.34), 4.320 (0.48), 4.400 (0.82), 4.418 (2.68), 4.428 (1.52), 4.949 (4.40), 5.383 (13.07), 5.536 (0.53), 5.553 (1.80), 5.568 (1.80), 5.585 (0.52), 6.115 (0.97), 6.128 (8.45), 6.397 (0.68), 7.081 (1.56), 7.083 (1.68), 7.093 (1.61), 7.095 (1.64), 7.270 (3.03), 7.331 (0.40), 8.107 (3.68), 8.111 (3.63), 8.121 (0.49), 8.126 (0.46), 8.510 (2.45), 8.523 (2.40).

### Example 682

### (3R)-2′-{6-amino-5-[(1R)-1-(6-methylpyridin-2-yl)ethoxy]pyridin-3-yl}-N-(propan-2-yl)-5′,6′-dihydrospiro[pyrrolidine-3,4′-pyrrolo[1,2-b]pyrazole]-1-carboxamide

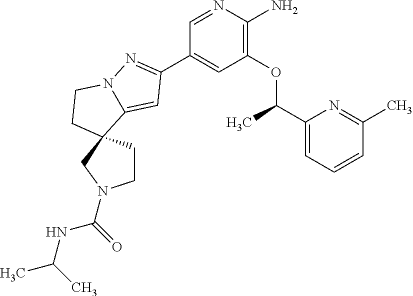 [SMILES: Cc1cccc([C@@H](C)Oc2cc(-c3cc4n(n3)CC[C@@]43CCN(C(=O)NC(C)C)C3)cnc2N)n1]

(3R)-1-[(Propan-2-yl)carbamoyl]-5′,6′-dihydrospiro[pyrrolidine-3,4′-pyrrolo[1,2-b]pyrazol]-2′-yl trifluoromethanesulfonate (37.6 mg, 94.9 μmol), {6-amino-5-[(1R)-1-(6-methylpyridin-2-yl)ethoxy]pyridin-3-yl}boronic acid (Intermediate 461, 110 mg, 33% purity, 133 μmol) and potassium phosphate (570 μL, 0.50 M, 280 μmol) were dissolved in degassed 1,4-dioxane (1.6 mL) under argon. XPhos Pd G2 (3.73 mg, 4.75 μmol) was added and the mixture was stirred for 2 h at 100° C. The reaction mixture was allowed to cool down, diluted with ethyl acetate and filtered. The filtrate was washed with water and brine, dried over sodium sulfate, filtered and concentrated. The residue was purified by preparative TLC (gradient: dichloromethane/methanol 9:1) to give 21.1 mg (98% purity, 46% yield) of the title compound.

LC-MS (Method 1): Rₜ=0.95 min; MS (ESIpos): m/z=477 [M+H]⁺

¹H-NMR (400 MHz, CHLOROFORM-d) δ [ppm]: 1.173 (7.23), 1.182 (9.11), 1.188 (9.64), 1.197 (7.09), 1.248 (0.58), 1.258 (0.40), 1.712 (7.78), 1.728 (7.95), 1.843 (0.84), 2.022 (0.46), 2.040 (0.67), 2.053 (1.10), 2.067 (0.96), 2.084 (0.67), 2.172 (0.62), 2.191 (1.29), 2.209 (0.83), 2.222 (0.91), 2.241 (0.46), 2.491 (0.40), 2.508 (0.91), 2.524 (1.12), 2.541 (1.44), 2.558 (1.12), 2.574 (16.00), 2.600 (1.58), 2.633 (0.88), 2.651 (0.44), 3.462 (0.86), 3.487 (3.02), 3.504 (4.21), 3.525 (1.78), 3.542 (0.69), 3.583 (0.62), 3.602 (0.97), 3.615 (0.85), 3.991 (2.69), 4.001 (1.92), 4.017 (1.09), 4.034 (0.55), 4.199 (2.30), 4.217 (4.07), 4.235 (2.16), 4.882 (4.87), 5.307 (3.35), 5.458 (0.58), 5.474 (1.82), 5.490 (1.81), 5.507 (0.57), 6.057 (6.45), 7.034 (2.21), 7.053 (2.37), 7.176 (2.09), 7.195 (2.32), 7.317 (3.33), 7.321 (3.41), 7.523 (1.68), 7.542 (2.96), 7.561 (1.38), 8.026 (3.42), 8.030 (3.59).

### Example 683

### (3R)-2′-{6-amino-5-[(1R)-1-(6-methylpyridin-2-yl)ethoxy]pyridin-3-yl}-N-cyclobutyl-5′,6′-dihydrospiro[pyrrolidine-3,4′-pyrrolo[1,2-b]pyrazole]-1-carboxamide

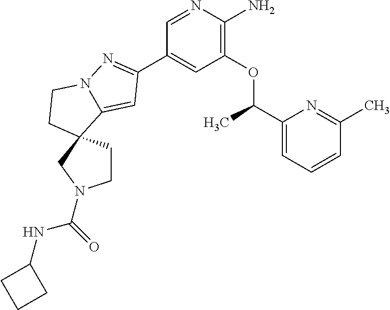 [SMILES: Cc1cccc([C@@H](C)Oc2cc(-c3cc4n(n3)CC[C@@]43CCN(C(=O)NC4CCC4)C3)cnc2N)n1]

(3R)-1-(Cyclobutylcarbamoyl)-5′,6′-dihydrospiro[pyrrolidine-3,4′-pyrrolo[1,2-b]pyrazol]-2′-yl trifluoromethanesulfonate (38.8 mg, 94.9 μmol), {6-amino-5-[(1R)-1-(6-methylpyridin-2-yl)ethoxy]pyridin-3-yl}boronic acid (Intermediate 461, 110 mg, 33% purity, 133 μmol) and potassium phosphate (570 μL, 0.50 M, 280 μmol) were dissolved in degassed 1,4-dioxane (1.6 mL) under argon. XPhos Pd G2 (3.73 mg, 4.75 μmol) was added and the mixture was stirred for 2 h at 100° C. The reaction mixture was allowed to cool down, diluted with ethyl acetate and filtered. The filtrate was washed with water and brine, dried over sodium sulfate, filtered and concentrated. The residue was purified by preparative TLC (gradient: dichloromethane/methanol 9:1) to give 24.7 mg (98% purity, 52% yield) of the title compound.

LC-MS (Method 1): Rₜ=0.97 min; MS (ESIpos): m/z=489 [M+H]⁺

¹H-NMR (400 MHz, CHLOROFORM-d) δ [ppm]: 1.651 (0.74), 1.660 (1.07), 1.677 (1.80), 1.684 (1.53), 1.693 (1.45), 1.705 (2.58), 1.712 (8.13), 1.727 (7.74), 1.808 (1.08), 1.823 (1.41), 1.836 (1.41), 2.019 (0.41), 2.036 (0.60), 2.050 (1.01), 2.064 (0.86), 2.081 (0.61), 2.167 (0.56), 2.186 (1.20), 2.198 (0.60), 2.204 (0.77), 2.217 (0.83), 2.235 (0.43), 2.351 (1.13), 2.361 (1.44), 2.370 (1.33), 2.375 (1.31), 2.387 (1.03), 2.501 (0.73), 2.517 (1.04), 2.535 (1.26), 2.551 (0.86), 2.573 (16.00), 2.592 (1.47), 2.643 (0.43), 3.463 (0.73), 3.488 (2.80), 3.504 (4.08), 3.528 (2.11), 3.545 (0.63), 3.582 (0.57), 3.596 (0.75), 3.601 (0.87), 3.615 (0.75), 4.195 (2.04), 4.214 (3.59), 4.231 (1.89), 4.324 (0.94), 4.340 (2.85), 4.882 (4.47), 5.306 (5.05), 5.458 (0.54), 5.473 (1.74), 5.490 (1.73), 5.506 (0.52), 6.054 (6.61), 7.034 (2.08), 7.053 (2.23), 7.176 (1.96), 7.195 (2.17), 7.315 (3.11), 7.319 (3.16), 7.522 (1.67), 7.541 (2.93), 7.561 (1.37), 8.026 (3.32), 8.031 (3.32).

### Example 684

### (3S)-2′-[6-amino-5-(trifluoromethyl)pyridin-3-yl]-N-[2-(3-fluoropyridin-2-yl) propan-2-yl]-5′,6′-dihydrospiro[pyrrolidine-3,4′-pyrrolo[1,2-b]pyrazole]-1-carboxamide

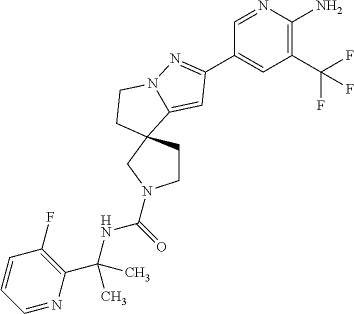 [SMILES: CC(C)(NC(=O)N1CC[C@]2(CCn3nc(-c4cnc(N)c(C(F)(F)F)c4)cc32)C1)c1ncccc1F]

2-(3-Fluoropyridin-2-yl)propan-2-amine (35.4 mg, 229 μmol), CDI (40.6 mg, 250 μmol) and N,N-diisopropylethylamine (150 μL, 830 μmol) were dissolved in DMF and stirred for 1 h at rt. A solution of 5-[(3S)-5′,6′-dihydrospiro[pyrrolidine-3,4′-pyrrolo[1,2-b]pyrazol]-2′-yl]-3-(trifluoromethyl)pyridin-2-amine-hydrogen chloride (75.0 mg, 208 μmol) in DMF was added dropwise. The mixture was stirred over night at rt, 2 h at 60° C., 2 h at 80° C. and 3 h at 100° C. The reaction mixture was allowed to cool down, filtered, and the concentrated filtrate was purified by preparative HPLC to give 23.0 mg (95% purity, 21% yield) of the title compound.

LC-MS (Method 1): Rₜ=1.07 min; MS (ESIpos): m/z=504 [M+H]⁺

¹H-NMR (400 MHz, CHLOROFORM-d) δ [ppm]: 1.696 (0.48), 1.712 (0.45), 1.854 (8.29), 1.858 (8.90), 1.863 (8.69), 1.867 (8.24), 2.106 (0.42), 2.110 (0.43), 2.123 (0.76), 2.137 (0.63), 2.155 (0.44), 2.247 (0.44), 2.266 (0.90), 2.278 (0.44), 2.285 (0.56), 2.297 (0.63), 2.581 (0.71), 2.597 (0.82), 2.631 (0.64), 2.678 (0.66), 2.696 (1.50), 2.713 (0.87), 2.729 (0.72), 2.746 (0.41), 3.580 (1.06), 3.605 (2.32), 3.629 (0.95), 3.641 (2.98), 3.654 (1.34), 3.666 (1.42), 3.672 (0.66), 3.722 (0.51), 3.736 (0.63), 3.742 (0.74), 3.747 (0.52), 3.755 (0.64), 3.760 (0.53), 3.767 (0.43), 4.272 (2.28), 4.290 (4.08), 4.307 (2.12), 4.994 (3.32), 6.236 (6.62), 6.985 (2.21), 7.235 (0.75), 7.243 (0.92), 7.246 (0.92), 7.255 (2.03), 7.260 (16.00), 7.266 (1.46), 7.276 (1.03), 7.384 (0.99), 7.387 (1.07), 7.404 (0.84), 7.407 (0.85), 7.413 (1.06), 7.416 (1.07), 7.433 (0.81), 7.436 (0.76), 8.108 (2.10), 8.112 (2.13), 8.307 (0.96), 8.310 (1.62), 8.314 (1.10), 8.318 (1.07), 8.322 (1.59), 8.326 (0.97), 8.602 (2.05), 8.606 (2.00).

### Example 685

### (3S)-2′-[6-amino-5-(trifluoromethyl)pyridin-3-yl]-N-[1-(1,3,5-trimethyl-1H-pyrazol-4-yl)ethyl]-5′,6′-dihydro-1H-spiro[pyrrolidine-3,4′-pyrrolo[1,2-b]pyrazole]-1-carboxamide (Diastereomer 1 and Diastereomer 2)

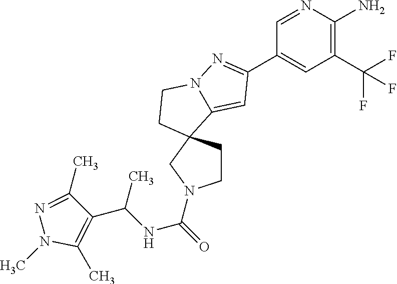 [SMILES: Cc1nn(C)c(C)c1C(C)NC(=O)N1CC[C@]2(CCn3nc(-c4cnc(N)c(C(F)(F)F)c4)cc32)C1]

1-(1,3,5-Trimethyl-1H-pyrazol-4-yl)ethan-1-amine (35.1 mg, 229 μmol), CDI ((40.6 mg, 250 μmol) and N,N-diisopropylethylamine (150 μL, 830 μmol) were dissolved in DMF and stirred for 1 h at rt. A solution of 5-[(3S)-5′,6′-dihydrospiro[pyrrolidine-3,4′-pyrrolo[1,2-b]pyrazol]-2′-yl]-3-(trifluoromethyl)pyridin-2-amine-hydrogen chloride (75.0 mg, 208 μmol) in DMF was added and the mixture was stirred over night at rt. The reaction mixture was filtered and the concentrated filtrate was purified by preparative HPLC followed by preparative TLC to give 6.10 mg (100% purity, 6% yield) of the title compound.

LC-MS (Method 1): Rₜ=0.92 min; MS (ESIpos): m/z=503 [M+H]⁺

¹H-NMR (400 MHz, CHLOROFORM-d) δ [ppm]: 0.075 (0.43), 0.868 (0.51), 0.886 (0.99), 0.901 (0.42), 1.240 (0.80), 1.259 (4.57), 1.290 (0.78), 1.316 (0.64), 1.489 (6.10), 1.496 (6.19), 1.506 (6.00), 1.513 (6.10), 1.991 (0.80), 2.029 (2.58), 2.062 (0.51), 2.076 (0.65), 2.080 (0.68), 2.093 (1.23), 2.097 (1.04), 2.107 (1.06), 2.111 (0.96), 2.125 (0.71), 2.179 (0.70), 2.207 (0.65), 2.214 (0.54), 2.227 (1.22), 2.233 (0.96), 2.240 (0.75), 2.245 (1.08), 2.259 (1.02), 2.267 (16.00), 2.274 (15.84), 2.293 (13.44), 2.303 (13.01), 2.547 (0.62), 2.556 (0.64), 2.563 (0.69), 2.572 (0.75), 2.580 (0.97), 2.589 (0.95), 2.597 (0.58), 2.628 (0.62), 2.639 (1.05), 2.645 (1.18), 2.658 (0.79), 2.663 (0.72), 2.673 (0.66), 2.678 (0.70), 3.478 (0.62), 3.501 (1.56), 3.525 (2.50), 3.532 (2.52), 3.540 (1.04), 3.546 (1.96), 3.557 (1.12), 3.571 (1.03), 3.609 (0.65), 3.623 (0.69), 3.630 (0.72), 3.643 (0.61), 3.654 (0.40), 3.679 (1.11), 3.687 (14.03), 3.696 (13.34), 4.252 (3.31), 4.270 (6.08), 4.287 (3.15), 4.368 (0.90), 4.375 (0.98), 4.383 (1.04), 4.391 (0.92), 4.894 (1.30), 4.911 (1.93), 4.929 (1.22), 5.024 (4.07), 6.186 (5.44), 6.189 (7.03), 8.098 (2.97), 8.103 (3.04), 8.587 (2.70), 8.592 (2.68).

### Example 686

### (3R)-2′-[6-amino-5-(trifluoromethyl)pyridin-3-yl]-N-[1-(1,3,5-trimethyl-1H-pyrazol-4-yl)ethyl]-5′,6′-dihydro-1H-spiro[pyrrolidine-3,4′-pyrrolo[1,2-b]pyrazole]-1-carboxamide (Diastereomer 1 and Diastereomer 2)

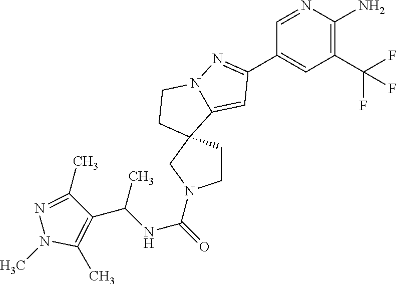 [SMILES: Cc1nn(C)c(C)c1C(C)NC(=O)N1CC[C@@]2(CCn3nc(-c4cnc(N)c(C(F)(F)F)c4)cc32)C1]

1-(1,3,5-Trimethyl-1H-pyrazol-4-yl)ethan-1-amine (37.5 mg, 245 μmol), CDI (43.3 mg, 267 μmol) and N,N-diisopropylethylamine (150 μL, 830 μmol) were dissolved in DMF and stirred for 1 h at rt. A solution of 5-[(3R)-5′,6′-dihydrospiro[pyrrolidine-3,4′-pyrrolo[1,2-b]pyrazol]-2′-yl]-3-(trifluoromethyl)pyridin-2-amine-hydrogen chloride (100 mg, 80% purity, 222 μmol) in DMF was added dropwise and the mixture was stirred over night at rt. The reaction mixture was diluted with water and extracted 3 times with ethyl acetate. The combined organic layers were dried over a hydrophobic filter and concentrated. The residue was purified by column chromatography (silica gel, dichloromethane/methanol (0-20%) gradient) to give 111 mg (90% purity, 89% yield) of the title compound.

LC-MS (Method 1): Rₜ=0.94 min; MS (ESIpos): m/z=503 [M+H]⁺

¹H-NMR (400 MHz, DMSO-d6) δ [ppm]: 1.217 (1.35), 1.234 (1.72), 1.244 (1.50), 1.262 (1.39), 1.322 (4.88), 1.330 (4.98), 1.339 (4.99), 1.348 (4.45), 1.475 (2.94), 1.494 (2.92), 1.999 (0.43), 2.015 (1.36), 2.032 (1.81), 2.040 (1.96), 2.057 (0.95), 2.071 (3.78), 2.096 (3.70), 2.106 (11.61), 2.115 (2.01), 2.126 (13.74), 2.150 (7.92), 2.153 (4.47), 2.178 (12.15), 2.186 (2.29), 2.194 (12.55), 2.229 (8.11), 2.331 (1.21), 2.336 (0.60), 2.518 (8.38), 2.523 (5.93), 2.673 (1.19), 2.678 (0.56), 3.160 (4.59), 3.171 (4.71), 3.371 (2.92), 3.383 (3.04), 3.397 (3.00), 3.419 (2.47), 3.429 (2.05), 3.444 (1.12), 3.455 (1.16), 3.466 (0.68), 3.481 (0.77), 3.497 (0.73), 3.517 (0.80), 3.532 (0.65), 3.551 (13.50), 3.558 (3.20), 3.564 (12.41), 3.571 (4.26), 3.578 (7.64), 4.101 (0.81), 4.115 (1.02), 4.132 (0.66), 4.155 (2.17), 4.172 (4.00), 4.190 (2.06), 4.679 (1.41), 4.696 (1.98), 4.714 (1.37), 4.826 (0.46), 4.844 (0.57), 4.861 (0.46), 5.903 (0.42), 5.922 (0.45), 5.943 (0.53), 6.073 (1.61), 6.086 (1.45), 6.436 (3.08), 6.445 (5.39), 6.542 (4.93), 6.995 (1.39), 6.999 (1.72), 7.001 (1.64), 7.013 (15.85), 7.015 (16.00), 7.402 (0.41), 7.407 (0.42), 7.643 (8.17), 7.708 (1.04), 7.712 (1.68), 7.715 (1.04), 8.004 (2.90), 8.242 (1.06), 8.244 (1.55), 8.247 (1.00), 8.575 (3.13), 8.592 (0.55).

### Example 687

### (3R)-2′-[6-amino-5-(trifluoromethyl)pyridin-3-yl]-N-[1-(1,3,5-trimethyl-1H-pyrazol-4-yl)ethyl]-5′,6′-dihydro-1H-spiro[pyrrolidine-3,4′-pyrrolo[1,2-b]pyrazole]-1-carboxamide (Diastereomer 1)

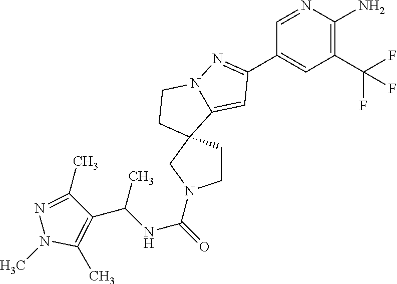 [SMILES: Cc1nn(C)c(C)c1C(C)NC(=O)N1CC[C@@]2(CCn3nc(-c4cnc(N)c(C(F)(F)F)c4)cc32)C1]

The title compound from Example 686 was separated into diastereomers by preparative chiral HPLC to give 36.0 mg (97% purity) of the title compound (diastereomer 1, Rₜ=3.5-4.5 min)

Preparative Chiral HPLC Method:

Instrument: Sepiatec Prep SFC100; Column: YMC Amylose SA 5μ, 250×30; eluent A: CO2; eluent B: 2-propanol+0.4 vol % diethylamin; isocratic: 30% B; flow: 100 mL/min; temperature: 40° C.; BPR: 150 bar; UV: 280 nm

Analytical Chiral HPLC Method:

Instrument: Agilent 1260, Aurora SFC-Modul; Column: YMC Amylose SA 3μ, 100×4.6; eluent A: CO2; eluent B: 2-propanol+0.4 vol % diethylamin; isocratic: 30% B; flow: 4 mL/min; temperature: 37.5° C.; BPR: 100 bar; UV: 280 nm

Analytical chiral HPLC: Rₜ=1.37 min.

LC-MS (Method 1): Rₜ=0.94 min; MS (ESIpos): m/z=503 [M+H]⁺

¹H-NMR (400 MHz, DMSO-d6) δ [ppm]: 0.846 (0.80), 0.853 (1.26), 0.859 (1.82), 0.861 (1.90), 0.864 (1.78), 0.871 (2.25), 0.887 (2.32), 0.906 (2.09), 0.925 (0.67), 1.277 (1.70), 1.286 (1.38), 1.330 (5.84), 1.348 (5.84), 1.368 (0.69), 1.387 (0.48), 1.409 (0.46), 2.016 (0.79), 2.031 (1.11), 2.039 (1.35), 2.057 (0.63), 2.106 (14.81), 2.178 (16.00), 2.518 (3.45), 2.523 (2.50), 2.994 (0.55), 3.358 (1.57), 3.363 (1.00), 3.371 (0.72), 3.383 (2.52), 3.407 (0.40), 3.429 (1.96), 3.455 (1.12), 3.518 (0.76), 3.526 (0.52), 3.532 (0.61), 3.552 (14.68), 3.669 (0.42), 3.695 (0.48), 3.702 (0.45), 4.155 (1.72), 4.165 (1.04), 4.172 (2.69), 4.190 (1.57), 4.248 (0.49), 4.260 (0.47), 4.678 (0.92), 4.696 (1.31), 4.714 (0.90), 6.073 (1.38), 6.090 (1.31), 6.445 (7.27), 6.540 (3.60), 7.832 (0.48), 7.852 (0.53), 8.004 (2.09), 8.009 (2.09), 8.213 (0.44), 8.233 (0.43), 8.255 (0.63), 8.258 (0.52), 8.577 (1.91), 8.581 (1.89).

### Example 688

### (3R)-2′-[6-amino-5-(trifluoromethyl)pyridin-3-yl]-N-[1-(1,3,5-trimethyl-1H-pyrazol-4-yl)ethyl]-5′,6′-dihydro-1H-spiro[pyrrolidine-3,4′-pyrrolo[1,2-b]pyrazole]-1-carboxamide (Diastereomer 2)

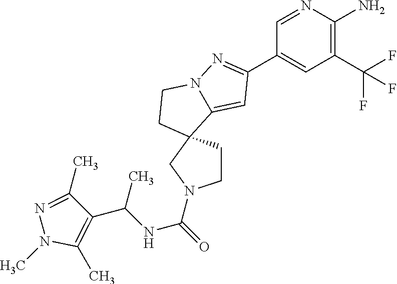 [SMILES: Cc1nn(C)c(C)c1C(C)NC(=O)N1CC[C@@]2(CCn3nc(-c4cnc(N)c(C(F)(F)F)c4)cc32)C1]

For the preparation of the diastereomeric mixture of the title compound see Example 686. The diastereomers were separated by preparative chiral HPLC (method see Example 687) to give 36.0 mg (95% purity) of the title compound (diastereomer 2, Rₜ=4.85-6.0 min).

Analytical chiral HPLC (method see Example 687): Rₜ=2.32 min.

LC-MS (Method 1): Rₜ=0.94 min; MS (ESIpos): m/z=503 [M+H]⁺

¹H-NMR (400 MHz, DMSO-d6) δ [ppm]: 0.845 (1.20), 0.852 (1.91), 0.858 (2.74), 0.861 (2.85), 0.863 (2.67), 0.871 (3.31), 0.887 (3.51), 0.905 (3.11), 0.924 (1.01), 1.231 (0.55), 1.277 (2.52), 1.286 (2.07), 1.322 (6.01), 1.340 (6.07), 1.368 (0.96), 1.387 (0.70), 1.409 (0.67), 1.427 (0.44), 1.460 (0.44), 2.016 (0.68), 2.033 (1.04), 2.041 (1.35), 2.059 (0.60), 2.126 (14.44), 2.195 (16.00), 2.518 (2.73), 2.523 (2.29), 2.994 (0.68), 3.371 (0.92), 3.380 (0.43), 3.397 (2.62), 3.420 (2.55), 3.445 (1.04), 3.464 (0.43), 3.482 (0.73), 3.490 (0.44), 3.497 (0.53), 3.508 (0.42), 3.564 (14.44), 3.645 (0.50), 3.670 (0.64), 3.695 (0.69), 3.702 (0.63), 4.155 (1.84), 4.164 (1.04), 4.173 (3.02), 4.184 (1.09), 4.191 (1.72), 4.248 (0.73), 4.259 (0.69), 4.679 (0.92), 4.697 (1.26), 4.714 (0.90), 6.069 (1.33), 6.085 (1.28), 6.436 (3.86), 6.544 (3.52), 7.831 (0.74), 7.851 (0.80), 8.000 (2.01), 8.006 (2.01), 8.208 (0.61), 8.213 (0.67), 8.228 (0.45), 8.232 (0.65), 8.255 (0.94), 8.258 (0.78), 8.571 (1.84), 8.574 (1.79).

### Example 689

### (2′-{6-amino-5-[(1R)-1-phenylethoxy]pyridin-3-yl}-5′,6′-dihydrospiro[azetidine-3,4′-pyrrolo[1,2-b]pyrazol]-1-yl)(cyclopropyl)methanone

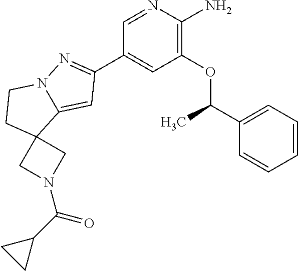 [SMILES: C[C@@H](Oc1cc(-c2cc3n(n2)CCC32CN(C(=O)C3CC3)C2)cnc1N)c1ccccc1]

5-(5′,6′-Dihydrospiro[azetidine-3,4′-pyrrolo[1,2-b]pyrazol]-2′-yl)-3-[(1R)-1-phenylethoxy]pyridin-2-amine-hydrogen chloride (Intermediate 520, 150 mg, 50% purity, 188 μmol) and cyclopropanecarboxylic acid (16.2 mg, 188 μmol) were dissolved in DMF (0.62 mL). N,N-Diisopropylethylamine (200 μL, 1.1 mmol) and 2,4,6-tripropyl-1,3,5,2,4,6-trioxatriphosphinane-2,4,6-trioxide (380 μL, 50% purity, 570 μmol) were added and the mixture was stirred for 1 h at rt. The reaction mixture was diluted with water and extracted with ethyl acetate. The combined organic layers were concentrated. The crude product was purified by preparative TLC and HPTLC (gradient: dichloromethane/methanol) to give 3.20 mg (90% purity, 4% yield) of the title compound.

LC-MS (Method 1): Rₜ=1.03 min; MS (ESIpos): m/z=430 [M+H]⁺

¹H-NMR (400 MHz, CHLOROFORM-d) δ [ppm]: 0.729 (1.28), 0.746 (1.36), 0.810 (0.45), 0.937 (0.45), 0.954 (1.15), 0.961 (1.18), 0.972 (0.54), 1.173 (10.06), 1.183 (2.68), 1.205 (0.51), 1.215 (0.42), 1.340 (0.40), 1.349 (0.49), 1.359 (0.70), 1.370 (0.43), 1.604 (6.35), 1.620 (6.41), 2.814 (1.48), 2.831 (2.02), 2.849 (1.57), 4.084 (0.48), 4.109 (0.76), 4.122 (1.21), 4.130 (0.57), 4.138 (2.09), 4.157 (1.14), 4.194 (0.54), 4.208 (0.55), 4.218 (0.41), 4.326 (0.53), 4.346 (0.72), 4.422 (0.52), 4.436 (0.58), 4.795 (2.34), 5.232 (3.61), 5.376 (1.13), 5.392 (1.12), 6.205 (1.13), 6.218 (1.12), 7.187 (2.03), 7.193 (16.00), 7.205 (1.15), 7.211 (0.49), 7.217 (0.55), 7.222 (1.06), 7.226 (0.64), 7.264 (1.05), 7.269 (0.57), 7.280 (1.00), 7.284 (2.78), 7.297 (0.55), 7.302 (2.38), 7.309 (2.64), 7.313 (3.12), 7.330 (1.09), 7.335 (0.73), 7.934 (2.56), 7.938 (2.54).

### Example 690

### 2′-[6-amino-5-(trifluoromethyl)pyridin-3-yl]-N-[1-(1-ethyl-1H-pyrazol-3-yl)ethyl]-5′,6′-dihydro-1H-spiro[azetidine-3,4′-pyrrolo[1,2-b]pyrazole]-1-carboxamide (Enantiomer 1)

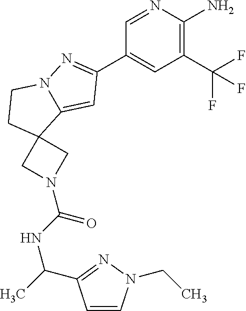 [SMILES: CCn1ccc(C(C)NC(=O)N2CC3(CCn4nc(-c5cnc(N)c(C(F)(F)F)c5)cc43)C2)n1]

1-(1-Ethyl-1H-pyrazol-3-yl)ethan-1-amine (67 μL, 30% purity, 160 μmol, CDI (28.1 mg, 174 μmol) and N,N-diisopropylethylamine (100 μL, 580 μmol) were dissolved in DMF and stirred for 1 h at rt. A solution of 5-(5′,6′-dihydrospiro[azetidine-3,4′-pyrrolo[1,2-b]pyrazol]-2′-yl)-3-(trifluoromethyl)pyridin-2-amine-hydrogen chloride (50.0 mg, 145 μmol) in DMF was added dropwise. The reaction mixture was stirred over night at rt. CDI (11.7 mg, 72 μmol) and N,N-diisopropylethylamine (12.7 μL, 72.5 μmol) were added again and the mixture was stirred for 2 h at rt. The reaction mixture was diluted with water and extracted 3 times with ethyl acetate. The combined organic layers were washed with brine, dried over a hydrophobic filter and concentrated. The residue was purified by column chromatography (silica gel, dichloromethane/methanol (0-15%) gradient) and preparative TLC (gradient: dichloromethane/methanol (9:1)) to give 16.1 mg (60% purity, 14% yield) of the title compound.

The enantiomers were separated by preparative chiral HPLC to give 2.00 mg (90% purity, 11% yield) of the title compound (enantiomer 1, Rₜ=7.8-8.8 min)

Preparative Chiral HPLC Method:

Instrument: PrepCon Labomatic HPLC-4; Column: YMC Cellulose SC 5p, 250×30; eluent A: methyl tert-butyl ether+0.1 vol % diethylamin; eluent B: ethanol; isocratic: 80% A+20% B; flow: 65 mL/min; temperature: 25° C.; UV: 280 nm

Analytical Chiral HPLC Method:

Instrument: Thermo Fisher UltiMate 3000; Column: YMC Cellulose SC 3μ, 100×4.6; eluent A: methyl tert-butyl ether+0.1 vol % diethylamin; eluent B: ethanol; isocratic: 80% A+20% B; flow: 1.4 mL/min; temperature: 25° C.; UV: 280 nm

Analytical chiral HPLC: Rₜ=4.39 min.

LC-MS (Method 1): Rₜ=0.92 min; MS (ESIpos): m/z=476 [M+H]⁺

¹H-NMR (400 MHz, CHLOROFORM-d) δ [ppm]: 0.759 (0.30), 0.774 (0.33), 0.783 (0.39), 0.793 (0.39), 0.800 (0.35), 0.811 (0.61), 0.827 (0.31), 1.124 (0.23), 1.141 (0.37), 1.159 (0.41), 1.185 (2.82), 1.215 (0.55), 1.231 (0.37), 1.264 (0.28), 1.288 (0.23), 1.306 (0.30), 1.324 (0.20), 1.354 (0.19), 1.358 (0.24), 1.368 (0.20), 1.379 (1.51), 1.387 (0.31), 1.397 (3.16), 1.404 (0.25), 1.416 (1.59), 1.444 (1.79), 1.461 (1.81), 2.825 (0.42), 2.842 (0.60), 2.860 (0.44), 3.424 (16.00), 4.031 (0.36), 4.049 (1.12), 4.053 (0.51), 4.058 (0.48), 4.067 (1.11), 4.073 (0.62), 4.078 (0.59), 4.085 (0.37), 4.138 (0.50), 4.156 (0.69), 4.165 (0.74), 4.169 (0.73), 4.173 (0.56), 4.184 (0.52), 4.188 (0.49), 4.861 (0.20), 4.879 (0.28), 4.950 (0.26), 4.967 (0.53), 4.974 (0.64), 4.984 (0.34), 6.068 (0.72), 6.074 (0.73), 6.355 (1.99), 7.251 (0.74), 7.256 (0.74), 8.044 (0.42), 8.048 (0.42), 8.531 (0.39), 8.535 (0.39).

### Example 691

### 2′-[6-amino-5-(trifluoromethyl)pyridin-3-yl]-N-[1-(1-ethyl-1H-pyrazol-3-yl)ethyl]-5′,6′-dihydro-1H-spiro[azetidine-3,4′-pyrrolo[1,2-b]pyrazole]-1-carboxamide (Enantiomer 2)

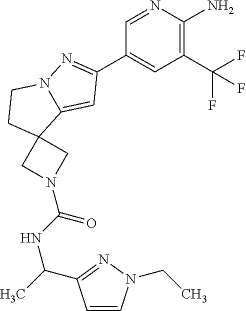 [SMILES: CCn1ccc(C(C)NC(=O)N2CC3(CCn4nc(-c5cnc(N)c(C(F)(F)F)c5)cc43)C2)n1]

For the preparation of the racemic mixture of the title compound see Example 690. The enantiomers were separated by preparative chiral HPLC (method see Example 690) to give 2.00 mg (90% purity, 11% yield) of the title compound (enantiomer 2, Rₜ=9.7-10.8 min).

Analytical chiral HPLC (method see Example 690): Rₜ=5.71 min.

LC-MS (Method 1): Rₜ=0.92 min; MS (ESpos): m/z=476 [M+H]⁺

¹H-NMR (400 MHz, CHLOROFORM-d) δ [ppm]: 0.720 (0.40), 0.836 (1.30), 0.852 (1.37), 0.860 (1.64), 0.870 (1.75), 0.877 (1.52), 0.887 (2.73), 0.903 (1.33), 0.929 (0.59), 0.946 (0.56), 1.081 (0.56), 1.098 (0.64), 1.106 (0.64), 1.218 (1.43), 1.262 (11.97), 1.292 (2.40), 1.308 (1.59), 1.340 (1.23), 1.369 (0.97), 1.387 (1.36), 1.405 (0.87), 1.431 (0.89), 1.435 (1.11), 1.455 (5.04), 1.474 (10.57), 1.492 (5.50), 1.521 (6.46), 1.538 (6.61), 1.610 (0.65), 1.649 (0.66), 1.665 (0.63), 2.356 (0.43), 2.902 (1.65), 2.919 (2.34), 2.937 (1.71), 3.501 (16.00), 4.108 (1.35), 4.126 (4.27), 4.130 (1.97), 4.135 (1.81), 4.144 (4.30), 4.150 (2.44), 4.155 (2.28), 4.162 (1.39), 4.215 (2.01), 4.224 (0.59), 4.233 (2.79), 4.241 (3.02), 4.245 (2.80), 4.250 (2.32), 4.260 (2.07), 4.264 (1.95), 4.943 (0.77), 4.961 (1.11), 5.026 (0.92), 5.043 (1.11), 5.061 (0.80), 5.087 (1.82), 6.146 (2.70), 6.152 (2.66), 6.431 (7.13), 6.445 (0.85), 7.328 (2.76), 7.333 (2.68), 7.528 (0.41), 8.124 (1.81), 8.129 (1.97), 8.603 (1.49), 8.608 (1.58).

### Example 692

### (3S)-2′-[6-amino-5-(trifluoromethyl)pyridin-3-yl]-N-[1-(1-ethyl-1H-pyrazol-3-yl)ethyl]-5′,6′-dihydro-1H-spiro[pyrrolidine-3,4′-pyrrolo[1,2-b]pyrazole]-1-carboxamide (Diastereomer 1)

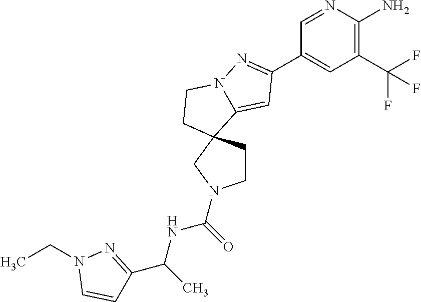 [SMILES: CCn1ccc(C(C)NC(=O)N2CC[C@]3(CCn4nc(-c5cnc(N)c(C(F)(F)F)c5)cc43)C2)n1]

1-(1-Ethyl-1H-pyrazol-3-yl)ethan-1-amine (64 μL, 30% purity, 150 μmol), CDI (27.0 mg, 167 μmol) and N,N-diisopropylethylamine (97 μL, 560 μmol) were dissolved in DMF and stirred for 1 h at rt. A solution of 5-[(3S)-5′,6′-dihydrospiro[pyrrolidine-3,4′-pyrrolo[1,2-b]pyrazol]-2′-yl]-3-(trifluoromethyl)pyridin-2-amine-hydrogen chloride (50.0 mg, 139 μmol) in DMF was added dropwise. The mixture was stirred over night at rt. CDI (11.2 mg, 69 μmol) was added again and the mixture was stirred for 2 h at rt. The reaction mixture was diluted with water and extracted 3 times with ethyl acetate. The combined organic layers were washed with brine, dried over a hydrophobic filter and concentrated. The residue was purified by column chromatography (silica gel, dichloromethane/methanol (0-15%) gradient) to give 43.0 mg (73% purity, 46% yield) of the title compound as a diastereomeric mixture.

The diastereomers were separated by preparative chiral HPLC to give 4.00 mg (95% purity, 9% yield) of the title compound (diastereomer 1, Rₜ=8.0-9.0 min)

Preparative Chiral HPLC Method:

Instrument: PrepCon Labomatic HPLC-4; Column: YMC Amylose SA 5μ, 250×30; eluent A: methyl tert-butyl ether+0.1 vol % diethylamin; eluent B: ethanol; isocratic: 80% A+20% B; flow: 50 mL/min; temperature: 25° C.; UV: 280 nm

Analytical Chiral HPLC Method:

Instrument: Thermo Fisher UltiMate 3000; Column: YMC Amylose SA 3μ, 100×4.6; eluent A: methyl tert-butyl ether+0.1 vol % diethylamin; eluent B: ethanol; isocratic: 80% A+20% B; flow: 1.4 mL/min; temperature: 25° C.; UV: 280 nm

Analytical chiral HPLC: Rₜ=3.65 min.

LC-MS (Method 1): Rₜ=0.95 min; MS (ESIpos): m/z=490 [M+H]⁺

¹H-NMR (400 MHz, CHLOROFORM-d) δ [ppm]: 0.759 (0.53), 0.774 (0.56), 0.783 (0.61), 0.793 (0.62), 0.800 (0.57), 0.811 (0.96), 0.827 (0.51), 1.185 (3.79), 1.206 (1.32), 1.213 (1.42), 1.231 (1.07), 1.354 (0.43), 1.363 (0.56), 1.380 (7.88), 1.397 (16.00), 1.416 (8.28), 1.448 (7.18), 1.464 (7.40), 1.942 (0.62), 1.998 (0.48), 2.015 (0.69), 2.029 (1.06), 2.043 (0.91), 2.061 (0.63), 2.144 (0.66), 2.163 (1.24), 2.175 (0.66), 2.181 (0.87), 2.193 (0.86), 2.212 (0.54), 2.470 (0.51), 2.487 (1.18), 2.502 (1.17), 2.520 (1.67), 2.537 (0.95), 2.578 (0.93), 2.596 (1.81), 2.629 (1.23), 2.647 (0.55), 3.474 (1.09), 3.481 (0.62), 3.498 (2.97), 3.524 (1.50), 3.535 (3.31), 3.559 (1.55), 3.586 (0.59), 3.606 (0.84), 3.620 (0.71), 4.031 (2.24), 4.050 (6.78), 4.068 (6.70), 4.087 (2.15), 4.183 (3.04), 4.201 (5.71), 4.218 (2.97), 4.935 (4.18), 4.963 (0.75), 4.984 (3.03), 4.999 (1.76), 5.016 (0.87), 6.067 (5.05), 6.072 (5.08), 6.136 (9.98), 7.250 (4.49), 7.255 (4.54), 8.028 (2.81), 8.033 (2.80), 8.520 (2.72), 8.524 (2.71).

### Example 693

### (3S)-2′-[6-amino-5-(trifluoromethyl)pyridin-3-yl]-N-[1-(1-ethyl-1H-pyrazol-3-yl)ethyl]-5′,6′-dihydro-1H-spiro[pyrrolidine-3,4′-pyrrolo[1,2-b]pyrazole]-1-carboxamide (Diastereomer 2)

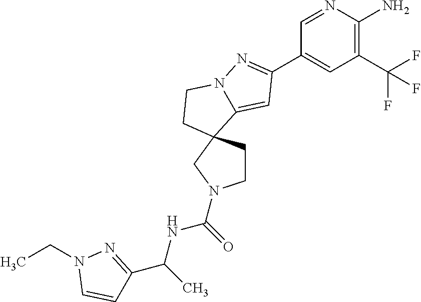 [SMILES: CCn1ccc(C(C)NC(=O)N2CC[C@]3(CCn4nc(-c5cnc(N)c(C(F)(F)F)c5)cc43)C2)n1]

For the preparation of the diastereomeric mixture of the title compound see Example 692. The diastereomer were separated by chiral HPLC (method see Example 692) to give 4.00 mg (95% purity, 9% yield) of the title compound (diastereomer 2, Rₜ=11.3-12.5 min).

Analytical chiral HPLC (method see Example 692): Rₜ=5.02 min.

LC-MS (Method 1): Rₜ=0.95 min; MS (ESIpos): m/z=490 [M+H]⁺

¹H-NMR (400 MHz, CHLOROFORM-d) δ [ppm]: 0.759 (0.65), 0.775 (0.68), 0.784 (0.76), 0.793 (0.80), 0.801 (0.73), 0.811 (1.23), 0.827 (0.63), 1.186 (4.70), 1.206 (1.51), 1.225 (1.21), 1.354 (0.62), 1.364 (1.00), 1.368 (7.73), 1.387 (16.00), 1.405 (7.51), 1.456 (5.51), 1.472 (5.61), 1.609 (0.48), 1.943 (0.58), 1.993 (0.45), 2.012 (0.71), 2.025 (1.04), 2.039 (0.86), 2.057 (0.60), 2.147 (0.61), 2.166 (1.14), 2.178 (0.60), 2.184 (0.75), 2.197 (0.79), 2.216 (0.44), 2.467 (0.44), 2.485 (0.97), 2.500 (1.02), 2.518 (1.58), 2.535 (0.81), 2.570 (0.89), 2.587 (1.82), 2.638 (0.48), 3.476 (0.91), 3.501 (2.64), 3.522 (3.22), 3.532 (1.40), 3.547 (1.29), 3.588 (0.58), 3.602 (0.74), 3.607 (0.85), 3.621 (0.72), 3.632 (0.48), 4.020 (2.08), 4.038 (6.29), 4.057 (6.23), 4.075 (1.98), 4.184 (2.71), 4.201 (5.35), 4.219 (2.60), 4.934 (3.97), 4.970 (1.03), 4.982 (3.34), 4.993 (1.91), 5.010 (0.70), 6.058 (4.62), 6.064 (4.81), 6.134 (9.60), 7.238 (4.36), 7.244 (4.47), 8.031 (2.65), 8.035 (2.70), 8.522 (2.55), 8.526 (2.53).

### Example 694

### 5-{(3′R)-1′-[cyclopropyl(1H-1,2,4-triazol-5-yl)methyl]-6,7-dihydrospiro[pyrazolo[5,1-c][1,4]oxazine-4,3′-pyrrolidin]-2-yl}-3-(trifluoromethyl)pyridin-2-amine (Diastereomer 1 and Diastereomer 2)

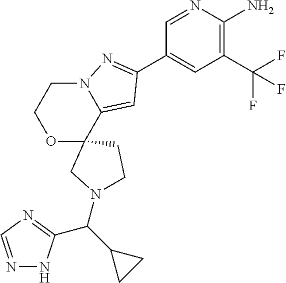 [SMILES: Nc1ncc(-c2cc3n(n2)CCO[C@@]32CCN(C(c3ncn[nH]3)C3CC3)C2)cc1C(F)(F)F]

5-[(3R)-6,7-Dihydrospiro[pyrazolo[5,1-c][1,4]oxazine-4,3-pyrrolidin]-2-yl]-3-(trifluoromethyl)pyridin-2-amine hydrogen chloride (1/1) (150 mg, 95% purity, 379 μmol) and cyclopropyl(1H-1,2,4-triazol-5-yl)methanone (78.0 mg, 569 μmol) were dissolved in methanol (4 mL). N,N-Diisopropylethylamine (260 μL, 1.5 mmol) and titanium(IV) isopropoxide (340 μL, 1.1 mmol) were added and the mixture was stirred for 1.5 h at 60° C. To the cold reaction mixture was added sodium cyanoborohydride (71.5 mg, 1.14 mmol) and the mixture was stirred over night at 60° C. The reaction mixture was allowed to cool down, diluted with water (0.5 mL) and stirred for 15 min. The suspension was filtered and the filter cake was washed with methanol. The filtrate was concentrated. The crude product was purified by preparative HPLC to give 45.7 mg (100% purity, 26% yield) of the title compound.

LC-MS (Method 2): Rₜ=0.67 min; MS (ESIpos): m/z=461 [M+H]⁺

¹H-NMR (400 MHz, DMSO-d₆) 5 [ppm]: 0.087 (0.93), 0.355 (1.35), 0.428 (0.85), 0.679 (0.87), 1.256 (0.92), 2.133 (1.21), 2.178 (1.10), 2.318 (1.07), 2.323 (2.40), 2.327 (3.49), 2.331 (2.50), 2.336 (1.15), 2.518 (16.00), 2.523 (10.65), 2.659 (2.26), 2.665 (3.69), 2.669 (4.76), 2.673 (3.63), 2.739 (1.49), 2.765 (1.61), 2.844 (2.22), 2.864 (2.45), 2.887 (1.44), 2.987 (0.94), 3.190 (0.76), 3.217 (0.93), 4.020 (2.65), 4.065 (4.28), 6.577 (7.11), 6.644 (1.61), 6.663 (1.04), 7.832 (1.28), 8.026 (3.98), 8.031 (6.41), 8.139 (7.05), 8.444 (0.71), 8.610 (4.13), 13.742 (0.58), 13.894 (1.38).

### Example 695

### 5-{(3′R)-1′-[cyclopropyl(1H-1,2,4-triazol-5-yl)methyl]-6,7-dihydrospiro[pyrazolo[5,1-c][1,4]oxazine-4,3′-pyrrolidin]-2-yl}-3-(trifluoromethyl)pyridin-2-amine (Diastereomer 1)

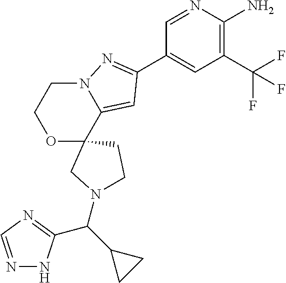 [SMILES: Nc1ncc(-c2cc3n(n2)CCO[C@@]32CCN(C(c3ncn[nH]3)C3CC3)C2)cc1C(F)(F)F]

The title compound from Example 694 was separated into diastereomers by preparative chiral HPLC to give 17.9 mg (99% purity, 40% yield) of the title compound (diastereomer 1 Rₜ=11.8-13.5 min).

Preparative Chiral HPLC Method:

Instrument: Sepiatec: Prep SFC100; Column: Chiral Art Cellulose SB 5μ, 250×50; eluent A: CO2; eluent B: 2-propanol+0.4 vol % diethylamine; isocratic: 15% B; flow: 100 mL/min; temperature: 40° C.; BPR: 150 bar; UV: 267 nm

Analytical Chiral HPLC Method:

Instrument: Waters Acquity UPC2 QDA; Column: Chiral Art Cellulose SB 3μ, 100×4.6; eluent A: CO2; eluent B: 2-propanol+0.4 vol % diethylamine; isocratic: 15% B; flow: 4 mL/min; temperature: 40° C.; BPR: 1800 psi; UV: 254 nm

Analytical chiral HPLC: Rₜ=3.00 min

[α]²⁰_{D}: +102.1° (c=1.00, methanol)

LC-MS (Method 1): Rₜ=0.81 min; MS (ESIpos): m/z=461 [M+H]⁺

¹H NMR (400 MHz, DMSO-d₆) δ ppm 0.01-0.12 (m, 1H), 0.30-0.47 (m, 2H), 0.59-0.72 (m, 1H), 1.20-1.34 (m, 1H), 2.04-2.23 (m, 2H), 2.61-2.69 (m, 1H), 2.83-3.03 (m, 4H), 3.92-4.13 (m, 4H), 6.52-6.70 (m, 3H), 7.74-7.97 (m, 1H), 8.03 (d, 1H), 8.61 (d, 1H), 13.65-13.97 (m, 1H).

### Example 696

### 5-{(3′R)-1′-[cyclopropyl(1H-1,2,4-triazol-5-yl)methyl]-6,7-dihydrospiro[pyrazolo[5,1-c][1,4]oxazine-4,3′-pyrrolidin]-2-yl}-3-(trifluoromethyl)pyridin-2-amine (Diastereomer 2)

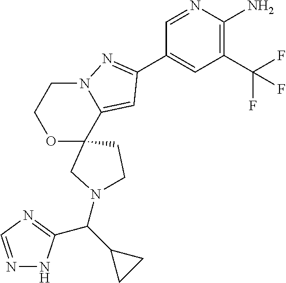 [SMILES: Nc1ncc(-c2cc3n(n2)CCO[C@@]32CCN(C(c3ncn[nH]3)C3CC3)C2)cc1C(F)(F)F]

For the preparation of the diastereomeric mixture of the title compound see Example 694. The diastereomers were separated by preparative chiral HPLC (method see Example 695) to give 8.50 mg (99% purity, 19% yield) of the title compound (diastereomer 2 Rₜ=14.2-16.6 min).

Analytical chiral HPLC (method see Example 695): Rₜ=3.74 min.

[α]²⁰_{D}: +37.3° (c=1.00, methanol)

LC-MS (Method 1): Rₜ=0.83 min; MS (ESIpos): m/z=461 [M+H]⁺

¹H NMR (400 MHz, DMSO-d6) δ ppm 0.01-0.12 (m, 1H), 0.30-0.47 (m, 2H), 0.59-0.72 (m, 1H), 1.20-1.34 (m, 1H), 2.04-2.23 (m, 2H), 2.61-2.69 (m, 1H), 2.83-3.03 (m, 4H), 3.92-4.13 (m, 4H), 6.52-6.70 (m, 3H), 7.74-7.97 (m, 1H), 8.03 (d, 1H), 8.61 (d, 1H), 13.65-13.97 (m, 1H).

### Example 697

### 5-{(3R)-1-[1-(1H-1,2,4-triazol-5-yl)propyl]-5′,6′-dihydrospiro[pyrrolidine-3,4′-pyrrolo[1,2-b]pyrazol]-2′-yl}-3-(trifluoromethyl)pyridin-2-amine (Diastereomer 1 and Diastereomer 2)

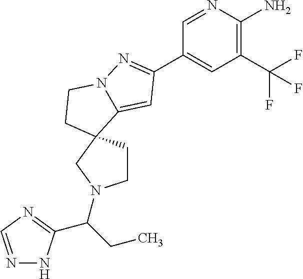 [SMILES: CCC(c1ncn[nH]1)N1CC[C@@]2(CCn3nc(-c4cnc(N)c(C(F)(F)F)c4)cc32)C1]

5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (50.0 mg, 95% purity, 132 μmol) and 1-(1H-1,2,4-triazol-5-yl)propan-1-one (24.8 mg, 198 μmol) were dissolved in methanol (1.4 mL). N,N-Diisopropylethylamine (92 μL, 530 μmol) and titanium(IV) isopropoxide (120 μL, 400 μmol) were added and the mixture was stirred for 3 h at 60° C. To the cold reaction mixture was added sodium cyanoborohydride (24.9 mg, 396 μmol) and the mixture was stirred for 2.5 h at 60° C. The reaction mixture was allowed to cool down, diluted with water (0.5 mL) and stirred for 15 min. The suspension was filtered and the filter cake was washed with methanol. The filtrate was concentrated. The crude product was purified by preparative HPLC to give 18.0 mg (96% purity, 30% yield) of the title compound.

LC-MS (Method 1): Rₜ=0.81 min; MS (ESIpos): m/z=433 [M+H]⁺

¹H-NMR (400 MHz, DMSO-d6) δ [ppm]: 0.755 (4.12), 0.759 (3.97), 0.773 (9.47), 0.777 (8.34), 0.792 (4.58), 0.932 (0.85), 0.948 (0.80), 1.794 (0.44), 1.812 (0.80), 1.827 (1.86), 1.847 (2.72), 1.866 (2.74), 1.877 (2.25), 1.893 (3.47), 1.909 (4.50), 1.927 (2.24), 2.318 (1.12), 2.323 (2.39), 2.327 (3.31), 2.331 (2.39), 2.336 (1.10), 2.378 (0.46), 2.396 (1.04), 2.411 (1.26), 2.428 (2.01), 2.439 (1.77), 2.446 (1.46), 2.458 (2.22), 2.518 (16.00), 2.523 (10.61), 2.605 (2.18), 2.627 (2.68), 2.634 (1.57), 2.660 (2.81), 2.665 (4.41), 2.673 (8.28), 2.699 (0.70), 2.766 (0.63), 2.787 (1.27), 2.802 (1.21), 2.813 (2.63), 2.835 (2.03), 2.849 (0.59), 2.865 (0.96), 2.888 (0.94), 2.905 (0.47), 3.165 (0.81), 3.681 (1.62), 3.699 (2.65), 3.718 (1.48), 4.057 (1.74), 4.066 (2.82), 4.075 (3.51), 4.084 (5.04), 4.101 (2.31), 6.291 (6.60), 6.329 (8.70), 6.513 (9.71), 7.992 (5.36), 8.104 (2.51), 8.552 (1.29), 8.565 (5.05).

### Example 698

### 5-{(3R)-1-[1-(1H-1,2,4-triazol-5-yl)propyl]-5′,6′-dihydrospiro[pyrrolidine-3,4′-pyrrolo[1,2-b]pyrazol]-2′-yl}-3-(trifluoromethyl)pyridin-2-amine (Diastereomer 1)

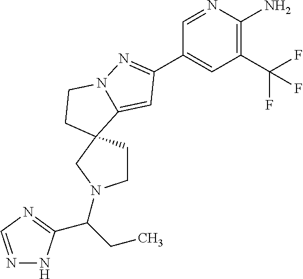 [SMILES: CCC(c1ncn[nH]1)N1CC[C@@]2(CCn3nc(-c4cnc(N)c(C(F)(F)F)c4)cc32)C1]

The title compound from Example 697 was separated into diastereomers by preparative chiral HPLC to give 17.7 mg (98% purity, 30% yield) of the title compound (diastereomer 1 Rₜ=9.44-12.39 min).

Preparative Chiral HPLC Method:

Instrument: PrepCon Labomatic HPLC-1; Column: YMC Amylose SA 10μ, 250×50; eluent A: hexane+0.1 vol % diethylamine; eluent B: ethanol; isocratic: 20% B; flow: 140 mL/min; temperature: 25° C.; UV: 280 nm

Analytical Chiral HPLC Method:

Instrument: Thermo Fisher UltiMate 3000; Column: YMC Amylose SA 3μ, 100×4.6; eluent A: hexane+0.1 vol % diethylamine; eluent B: ethanol; isocratic: 20% B; flow: 1.4 mL/min; temperature: 25° C.; UV: 280 nm

Analytical chiral HPLC: Rₜ=2.01 min

LC-MS (Method 2): Rₜ=0.65 min; MS (ESIpos): m/z=433 [M+H]⁺

¹H-NMR (400 MHz, DMSO-d6) δ [ppm]: 0.756 (7.34), 0.774 (16.00), 0.793 (7.99), 0.814 (1.27), 0.821 (0.98), 0.836 (0.75), 0.862 (0.70), 0.886 (0.43), 0.904 (0.65), 1.005 (0.87), 1.034 (0.76), 1.071 (0.44), 1.084 (1.82), 1.204 (0.47), 1.232 (1.90), 1.259 (2.61), 1.352 (14.18), 1.392 (0.41), 1.741 (0.55), 1.757 (1.55), 1.774 (0.74), 1.832 (2.40), 1.850 (3.99), 1.868 (4.16), 1.884 (3.74), 1.903 (4.35), 1.917 (3.75), 2.181 (2.03), 2.322 (2.72), 2.326 (3.56), 2.331 (2.63), 2.404 (1.66), 2.419 (2.30), 2.435 (3.38), 2.454 (3.46), 2.522 (15.61), 2.602 (2.59), 2.624 (2.95), 2.664 (4.79), 2.669 (5.18), 2.673 (4.26), 2.764 (1.36), 2.786 (2.66), 2.810 (4.96), 2.833 (3.47), 3.565 (0.97), 3.582 (0.55), 3.599 (1.20), 3.615 (0.53), 3.690 (1.66), 3.707 (2.74), 3.725 (1.61), 4.070 (5.22), 4.087 (10.43), 4.104 (5.00), 6.338 (8.74), 6.512 (13.19), 6.652 (0.57), 6.867 (1.26), 7.992 (7.78), 7.997 (7.72), 8.567 (7.27), 8.570 (7.11), 13.808 (2.04).

### Example 699

### 5-{(3R)-1-[1-(1H-1,2,4-triazol-5-yl)propyl]-5′,6′-dihydrospiro[pyrrolidine-3,4′-pyrrolo[1,2-b]pyrazol]-2′-yl}-3-(trifluoromethyl)pyridin-2-amine (Diastereomer 2)

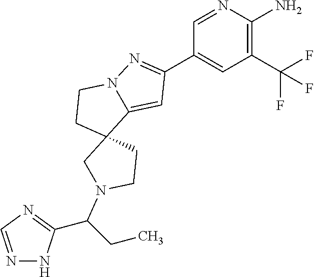 [SMILES: CCC(c1ncn[nH]1)N1CC[C@@]2(CCn3nc(-c4cnc(N)c(C(F)(F)F)c4)cc32)C1]

For the preparation of the diastereomeric mixture of the title compound see Example 697. The diastereomers were separated by preparative chiral HPLC (method see Example 698) to give 19.9 mg (92% purity, 32% yield) of the title compound (diastereomer 2 Rₜ=12.77-16.93 min).

Analytical chiral HPLC (method see Example 698): Rₜ=2.54 min.

LC-MS (Method 2): Rₜ=0.81 min; MS (ESIpos): m/z=433 [M+H]⁺

¹H-NMR (400 MHz, DMSO-d6) δ [ppm]: 0.760 (6.42), 0.778 (14.04), 0.797 (7.87), 0.814 (2.30), 0.821 (1.97), 0.840 (1.17), 0.862 (0.90), 0.886 (0.88), 0.904 (1.61), 0.922 (0.81), 1.005 (1.08), 1.034 (1.02), 1.053 (0.54), 1.071 (0.85), 1.083 (2.20), 1.159 (0.58), 1.204 (0.52), 1.233 (1.66), 1.259 (3.16), 1.352 (1.61), 1.391 (0.43), 1.741 (0.44), 1.757 (1.33), 1.774 (0.69), 1.798 (0.70), 1.816 (1.13), 1.831 (1.81), 1.851 (2.62), 1.872 (2.95), 1.903 (3.68), 1.916 (5.43), 1.933 (3.11), 2.322 (2.35), 2.326 (3.12), 2.331 (2.31), 2.394 (0.88), 2.413 (1.74), 2.428 (2.18), 2.446 (3.60), 2.522 (12.52), 2.612 (1.22), 2.631 (2.64), 2.654 (3.45), 2.669 (16.00), 2.850 (0.95), 2.867 (2.00), 2.888 (1.83), 2.905 (0.76), 3.565 (0.83), 3.582 (0.47), 3.599 (0.99), 3.615 (0.44), 3.704 (2.10), 4.061 (3.87), 4.078 (7.53), 4.096 (3.92), 6.306 (6.63), 6.513 (11.50), 7.988 (6.67), 7.993 (6.79), 8.561 (6.18), 8.565 (6.18), 13.811 (1.62).

### Example 700

### 5-{(3R)-1-[cyclopropyl(1H-1,2,4-triazol-5-yl)methyl]-5′,6′-dihydrospiro[pyrrolidine-3,4′-pyrrolo[1,2-b]pyrazol]-2′-yl}-3-(trifluoromethyl)pyridin-2-amine (Diastereomer 1 and Diastereomer 2)

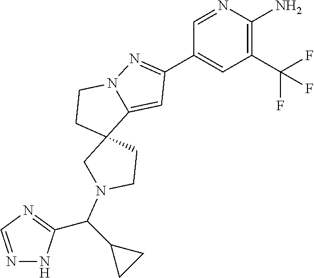 [SMILES: Nc1ncc(-c2cc3n(n2)CC[C@@]32CCN(C(c3ncn[nH]3)C3CC3)C2)cc1C(F)(F)F]

5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (150 mg, 95% purity, 396 μmol) and cyclopropyl(1H-1,2,4-triazol-5-yl)methanone (81.5 mg, 594 μmol) were dissolved in methanol (4.2 mL). N,N-Diisopropylethylamine (280 μL, 1.6 mmol) and titanium(IV) isopropoxide (350 μL, 1.2 mmol) were added and the mixture was stirred for 1.5 h at 60° C. To the cold reaction mixture was added sodium cyanoborohydride (74.7 mg, 1.19 mmol) and the mixture was stirred for 16 h at 60° C. The reaction mixture was allowed to cool down, diluted with water (0.5 mL) and stirred for 15 min. The suspension was filtered and the filter cake was washed with methanol. The filtrate was concentrated. The crude product was purified by preparative HPLC to give 44.0 mg (100% purity, 25% yield) of the title compound.

LC-MS (Method 1): Rₜ=0.83 min; MS (ESIpos): m/z=445 [M+H]⁺

¹H-NMR (400 MHz, DMSO-d6) δ [ppm]: 0.090 (0.47), 0.310 (0.62), 0.531 (0.44), 0.676 (0.61), 1.232 (0.62), 2.074 (1.27), 2.327 (4.25), 2.331 (3.10), 2.336 (1.48), 2.518 (16.00), 2.523 (11.04), 2.577 (0.63), 2.669 (4.43), 2.673 (3.19), 2.678 (1.53), 4.122 (1.50), 6.454 (0.77), 6.542 (3.86), 6.578 (1.75), 7.993 (3.06), 8.132 (0.58), 8.579 (3.24).

### Example 701

### 5-{(3′R)-1′-[1-(1H-1,2,4-triazol-5-yl)propyl]-6,7-dihydrospiro[pyrazolo[5,1-c][1,4]oxazine-4,3′-pyrrolidin]-2-yl}-3-(trifluoromethyl)pyridin-2-amine (Diastereomer 1 and Diastereomer 2)

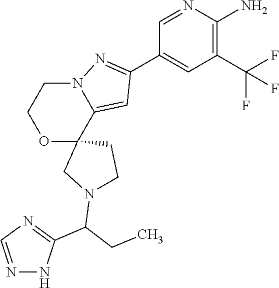 [SMILES: CCC(c1ncn[nH]1)N1CC[C@]2(C1)OCCn1nc(-c3cnc(N)c(C(F)(F)F)c3)cc12]

5-[(3′R)-6,7-Dihydrospiro[pyrazolo[5,1-c][1,4]oxazine-4,3′-pyrrolidin]-2-yl]-3-(trifluoromethyl)pyridin-2-amine hydrogen chloride (1/1) (150 mg, 85% purity, 339 μmol) and 1-(1H-1,2,4-triazol-5-yl)propan-1-one (63.7 mg, 509 μmol) were dissolved in methanol (3.6 mL). N,N-Diisopropylethylamine (240 μL, 1.4 mmol) and titanium(IV) isopropoxide (300 μL, 1.0 mmol) were added and the mixture was stirred for 3 h at 60° C. To the cold reaction mixture was added sodium cyanoborohydride (64.0 mg, 1.02 mmol) and the mixture was stirred for 2 h at 60° C. The reaction mixture was allowed to cool down, diluted with water (0.5 mL) and stirred for 15 min. The suspension was filtered and the filter cake was washed with methanol. The filtrate was concentrated. The crude product was purified by preparative HPLC to give a first batch of 21.0 mg (86% purity, 12% yield) and a second batch of 49.0 mg (91% purity, 29% yield) of the title compound.

LC-MS (Method 1): Rₜ=0.77 min; MS (ESIpos): m/z=449 [M+H]⁺

¹H-NMR (400 MHz, DMSO-d6) δ [ppm]: 0.705 (0.56), 0.726 (3.99), 0.731 (3.57), 0.745 (9.23), 0.750 (7.53), 0.763 (4.44), 0.768 (3.43), 0.917 (0.94), 0.932 (8.14), 0.934 (2.40), 0.948 (8.00), 0.952 (1.24), 1.788 (0.42), 1.805 (0.61), 1.821 (1.42), 1.840 (2.33), 1.858 (2.50), 1.875 (1.84), 1.893 (0.71), 2.003 (0.67), 2.020 (1.27), 2.036 (1.53), 2.052 (1.88), 2.074 (1.29), 2.081 (1.44), 2.099 (1.63), 2.113 (1.05), 2.131 (0.60), 2.318 (1.35), 2.323 (2.89), 2.327 (4.12), 2.331 (2.96), 2.336 (1.35), 2.386 (0.42), 2.404 (1.02), 2.421 (1.06), 2.440 (0.64), 2.518 (16.00), 2.523 (11.08), 2.619 (0.55), 2.638 (1.52), 2.659 (3.27), 2.665 (3.72), 2.669 (4.69), 2.673 (3.82), 2.678 (2.23), 2.784 (2.36), 2.809 (3.38), 2.818 (2.06), 2.830 (1.21), 2.840 (1.25), 2.865 (1.05), 2.891 (1.92), 2.924 (2.39), 2.941 (0.80), 2.950 (1.20), 2.957 (0.93), 2.974 (0.62), 3.052 (2.33), 3.078 (1.99), 3.165 (1.74), 3.640 (0.94), 3.654 (1.85), 3.668 (1.79), 3.674 (1.84), 3.689 (1.16), 3.962 (2.80), 3.974 (4.15), 3.987 (2.90), 4.042 (3.84), 4.051 (4.92), 6.436 (0.54), 6.471 (4.52), 6.504 (7.36), 6.568 (8.65), 7.620 (0.46), 8.018 (5.46), 8.023 (5.63), 8.083 (3.54), 8.589 (4.96).

### Example 702

### 5-{(3′R)-1′-[1-(1H-1,2,4-triazol-5-yl)propyl]-6,7-dihydrospiro[pyrazolo[5,1-c][1,4]oxazine-4,3′-pyrrolidin]-2-yl}-3-(trifluoromethyl)pyridin-2-amine (Diastereomer 1)

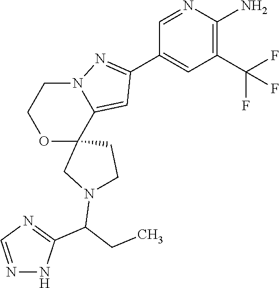 [SMILES: CCC(c1ncn[nH]1)N1CC[C@]2(C1)OCCn1nc(-c3cnc(N)c(C(F)(F)F)c3)cc12]

The title compound from Example 701 was separated into diastereomers by preparative chiral HPLC to give 39.0 mg (99% purity, 61% yield) of the title compound (diastereomer 1 Rₜ=14.0-15.9 min).

Preparative Chiral HPLC Method:

Instrument: PrepCon Labomatic HPLC-3; Column: YMC Amylose SB 5μ, 250×50; eluent A: hexane+0.1 vol % diethylamine; eluent B: ethanol; isocratic: 10% B; flow: 50 mL/min; temperature: 25° C.; UV: 280 nm

Analytical Chiral HPLC Method:

Instrument: Thermo Fisher UltiMate 3000; Column: YMC Amylose SB 3μ, 100×4.6; eluent A: hexane+0.1 vol % diethylamine; eluent B: ethanol; isocratic: 10% B; flow: 1.4 mL/min; temperature: 25° C.; UV: 280 nm

Analytical chiral HPLC: Rₜ=5.83 min

LC-MS (Method 1): Rₜ=0.77 min; MS (ESIpos): m/z=449 [M+H]⁺

¹H NMR (400 MHz, DMSO-d₆) δ ppm 0.75 (t, 3H), 1.78-1.96 (m, 2H), 1.99-2.18 (m, 2H), 2.61-2.69 (m, 1H), 2.75-2.86 (m, 2H), 3.07 (br d, 1H), 3.64-3.73 (m, 1H), 3.93-4.01 (m, 2H), 4.02-4.09 (m, 2H), 6.47-6.61 (m, 3H), 7.88 and 8.45 (2 br s, 1H), 8.02 (d, 1H), 8.59 (d, 1H), 13.83 (br s, 1H).

### Example 703

5-{(3′R)-1′-[1-(1H-1,2,4-Triazol-5-yl)propyl]-6,7-dihydrospiro[pyrazolo[5,1-c][1,4]oxazine-4,3′-pyrrolidin]-2-yl}-3-(trifluoromethyl)pyridin-2-amine (Diastereomer 2)

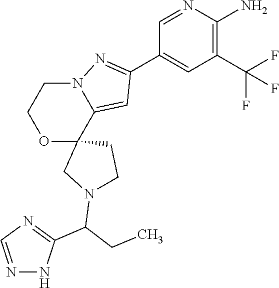 [SMILES: CCC(c1ncn[nH]1)N1CC[C@]2(C1)OCCn1nc(-c3cnc(N)c(C(F)(F)F)c3)cc12]

For the preparation of the diastereomeric mixture of the title compound see Example 701. The diastereomers were separated by preparative chiral HPLC (method see Example 702) to give 36.0 mg (98% purity, 56% yield) of the title compound (diastereomer 2 Rₜ=16.9-19.3 min).

Analytical chiral HPLC (method see Example 702): Rₜ=7.16 min.

LC-MS (Method 1): Rₜ=0.77 min; MS (ESIpos): m/z=449 [M+H]⁺

¹H NMR (400 MHz, DMSO-d₆) δ ppm 0.75 (br s, 3H), 1.78-1.95 (m, 2H), 1.96-2.20 (m, 2H), 2.62-2.71 (m, 1H), 2.78-2.95 (m, 3H), 3.59-3.73 (m, 1H), 3.90-4.10 (m, 4H), 6.45-6.62 (m, 3H), 7.89 and 8.47 (2s, 1H), 8.02 (d, 1H), 8.59 (s, 1H), 13.80 and 13.87 (2s, 1H).

### Example 704

### 5-{(3′R)-1′-[2,2-difluoro-1-(1H-imidazol-2-yl)ethyl]-6,7-dihydrospiro[pyrazolo[5,1-c][1,4]oxazine-4,3′-pyrrolidin]-2-yl}-3-(trifluoromethyl)pyridin-2-amine (Diastereomer 2)

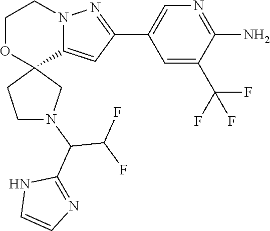 [SMILES: Nc1ncc(-c2cc3n(n2)CCO[C@@]32CCN(C(c3ncc[nH]3)C(F)F)C2)cc1C(F)(F)F]

5-[(3′R)-6,7-Dihydrospiro[pyrazolo[5,1-c][1,4]oxazine-4,3′-pyrrolidin]-2-yl]-3-(trifluoromethyl)pyridin-2-amine hydrogen chloride (1/1) (400 mg, 1.06 mmol) and 2,2-difluoro-1-(1H-Imidazol-2-yl)ethan-1-one (233 mg, 1.60 mmol) were dissolved in methanol (12 mL). N,N-Diisopropylethylamine (740 μL, 4.3 mmol) and titanium(IV) isopropoxide (940 μL, 3.2 mmol) were added and the mixture was stirred for 3 h at 60° C. To the cold reaction mixture was added sodium cyanoborohydride (201 mg, 3.19 mmol) and the mixture was stirred for 16 h at 60° C. Titanium(IV) isopropoxide (626 μL, 2.1 mmol) and sodium cyanoborohydride (134 mg, 2.1 mmol) were added again and the mixture was stirred for 22 h at 60° C. The reaction mixture was allowed to cool down, diluted with water and stirred for 15 min. The suspension was filtered and the filter cake was washed with methanol. The filtrate was concentrated. The residue was purified by column chromatography (silica gel NH2, hexane/ethyl acetate, 0-100% gradient). The combined fractions were purified by preparative HPLC to give 2.8 mg of the title compound as a mixture of diastereomers. The diastereomers were separated by preparative chiral HPLC to give 3.90 mg (80% purity, 1% yield) of the title compound (diastereomer 2 Rₜ=13.8-15.6 min).

Preparative Chiral HPLC Method:

Instrument: PrepCon Labomatic HPLC-3; Column: YMC Amylose SA 5μ, 250×50; eluent A: hexane+0.1 vol % diethylamine; eluent B: ethanol; isocratic: 20% B; flow: 50 mL/min; temperature: 25° C.; UV: 280 nm

Analytical Chiral HPLC Method:

Instrument: Thermo Fisher UltiMate 3000; Column: YMC Cellulose SA 3μ, 100×4.6; eluent A: hexane+0.1 vol % diethylamine; eluent B: ethanol; isocratic: 50% B; flow: 1.4 mL/min; temperature: 25° C.; UV: 280 nm

Analytical chiral HPLC: Rₜ=3.84 min

LC-MS (Method 1): Rₜ=0.93 min; MS (ESIpos): m/z=470 [M+H]⁺

¹H NMR (400 MHz, DMSO-d₆) δ ppm: 12.14 (br s, 1H), 8.59 (d, 1H), 8.01 (d, 1H), 7.16 (s, 1H), 6.92 (s, 1H), 6.58 (s, 2H), 6.48 (s, 1H), 6.41 (dt, 1H), 3.93-4.19 (m, 5H), 2.89-3.06 (m, 3H), 2.74 (td, 1H), 2.12-2.21 (m, 1H), 2.02-2.10 (m, 1H).

### Example 705

### 5-{(3′R)-1′-[2,2-difluoro-1-(1H-imidazol-2-yl)ethyl]-6,7-dihydrospiro[pyrazolo[5,1-c][1,4]oxazine-4,3′-pyrrolidin]-2-yl}-3-(trifluoromethyl)pyridin-2-amine (Diastereomer 1)

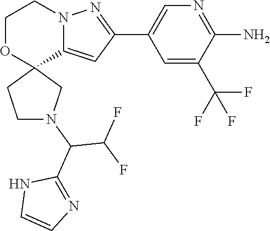 [SMILES: Nc1ncc(-c2cc3n(n2)CCO[C@@]32CCN(C(c3ncc[nH]3)C(F)F)C2)cc1C(F)(F)F]

For the preparation of the diastereomeric mixture of the title compound see Example 704. The diastereomers were separated by preparative chiral HPLC (method see Example 704) to give 8.10 mg (80% purity, 1% yield) of the title compound (diastereomer 1 Rₜ=7.9-9.1 min).

Analytical chiral HPLC (method see Example 704): Rₜ=2.21 min

LC-MS (Method 1): Rₜ=0.93 min; MS (ESIpos): m/z=470 [M+H]⁺

¹H NMR (400 MHz, DMSO-d₆) δ ppm: 12.13 (br s, 1H), 8.59 (d, 1H), 8.01 (d, 1H), 7.14-7.16 (m, 1H), 6.92 (t, 1H), 6.58 (s, 2H), 6.54 (s, 1H), 6.40 (dt, 1H), 4.03-4.16 (m, 3H), 3.91-4.02 (m, 2H), 3.19 (d, 1H), 2.94 (td, 1H), 2.86 (d, 1H), 2.71-2.79 (m, 1H), 2.02-2.20 (m, 2H).

### Example 706

### 5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}-3-(difluoromethyl)pyridin-2-amine (Diastereomer 1 and Diastereomer 2)

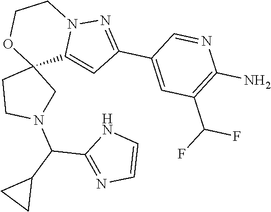 [SMILES: Nc1ncc(-c2cc3n(n2)CCO[C@@]32CCN(C(c3ncc[nH]3)C3CC3)C2)cc1C(F)F]

3-(Difluoromethyl)-5-[(3′R)-6,7-dihydrospiro[pyrazolo[5,1-c][1,4]oxazine-4,3′-pyrrolidin]-2-yl]pyridin-2-amine (130 mg, 405 μmol, Intermediate 523) and cyclopropyl(1H-imidazol-2-yl)methanone (66.1 mg, 485 μmol, CAS 952200-96-3) were dissolved in methanol (1.4 mL). N,N-Diisopropylethylamine (350 μL, 2.0 mmol) and titanium(IV) isopropoxide (360 μL, 1.2 mmol) were added and the mixture was stirred for 16 h at 60° C. The suspension was cooled to room temperature and sodium cyanoborohydride (76.3 mg, 1.21 mmol) was added and the mixture was stirred over the weekend at 60° C. The reaction mixture was allowed to cool down, diluted with water (1 mL) and stirred for 30 min. The suspension was diluted with methanol and filtered over celite. The filtrate was concentrated. The residue was dissolved in dichloromethane/methanol (small amount of methanol) and washed twice with saturated sodium hydrogen carbonate solution. The aqueous phase was extracted with dichloromethane/methanol. The combined organic layers were dried over an hydrophic filter and concentrated under reduced pressure. The crude product was purified by column chromatography (silica gel, dichloromethane/methanol (0-10%) gradient) to give 57.0 mg (94% purity, 30% yield) of the title compound.

LC-MS (Method 1): Rₜ=0.86 min; MS (ESIpos): m/z=442 [M+H]⁺

¹H NMR (400 MHz, CHLOROFORM-d) δ ppm: 8.52-8.56 (m, 1H), 7.92-7.96 (m, 1H), 7.00 (2s, 2H), 6.62 and 6.61 (2t, 1H), 6.29 and 6.28 (2s, 1H), 4.97 (br d, 2H), 4.15-4.22 (m, 2H), 4.04-4.14 (m, 2H), 3.46 (d, 1H, one diastereomer), 3.25-3.32 (m, 1H, one diastereomer), 2.92-3.04 (m, 1H), 2.65-2.90 (m, 3H), 2.20-2.43 (m, 2H), 1.04-1.17 (m, 1H), 0.80-0.91 (m, 1H), 0.68-0.77 (m, 1H), 0.42-0.52 (m, 2H), 0.33-0.42 (m, 1H)

### Example 707

### 5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}-3-(difluoromethyl)pyridin-2-amine (Diastereomer 1)

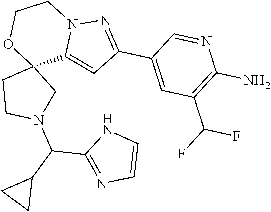 [SMILES: Nc1ncc(-c2cc3n(n2)CCO[C@@]32CCN(C(c3ncc[nH]3)C3CC3)C2)cc1C(F)F]

The title compound from Example 706 was separated into diastereomers by preparative chiral HPLC to give 14.1 mg (98% purity) of the title compound (diastereomer 1 Rₜ=6.0-7.2 min).

Preparative Chiral HPLC Method:

Instrument: Sepiatec: Prep SFC100; Column: Chiral Art Amylose SA 5μ, 250×50; eluent A: CO2; eluent B: 2-propanol+0.4 vol % diethylamine; isocratic: 30% B; flow: 100 mL/min; temperature: 40° C.; BPR: 150 bar UV: 267 nm

Analytical Chiral HPLC Method:

Instrument: Waters Acquity UPC2 QDA; Column: Chiral Art Amylose SA 3μ, 100×4.6; eluent A: CO2; eluent B: 2-propanol+0.4 vol % diethylamine; isocratic: 30% B; flow: 4 mL/min; temperature: 40° C.; BPR: 1800 psi; UV: 254 nm

Analytical chiral HPLC: Rₜ=1.56 min

LC-MS (Method 1): Rₜ=0.86 min; MS (ESIpos): m/z=442 [M+H]⁺

### Example 708

### 5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}-3-(difluoromethyl)pyridin-2-amine (Diastereomer 2)

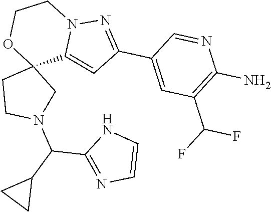 [SMILES: Nc1ncc(-c2cc3n(n2)CCO[C@@]32CCN(C(c3ncc[nH]3)C3CC3)C2)cc1C(F)F]

For the preparation of the diastereomeric mixture of the title compound see Example 706. The diastereomers were separated by preparative chiral HPLC (method see Example 707) to give 13.5 mg (99% purity) of the title compound (diastereomer 2 Rₜ=11.1-13.0 min).

Analytical chiral HPLC (method see Example 707): Rₜ=2.98 min.

LC-MS (Method 1): Rₜ=0.86 min; MS (ESIpos): m/z=442 [M+H]⁺

### Example 709

### 2-amino-5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}nicotinonitrile (Diastereomer 1 and Diastereomer 2)

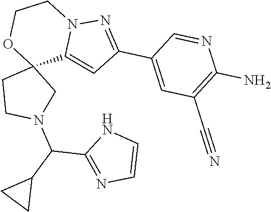 [SMILES: N#Cc1cc(-c2cc3n(n2)CCO[C@@]32CCN(C(c3ncc[nH]3)C3CC3)C2)cnc1N]

The compound was prepared similarly to Example 706 using 2-amino-5-[(3′R)-6,7-dihydrospiro[pyrazolo[5,1-c][1,4]oxazine-4,3′-pyrrolidin]-2-yl]pyridine-3-carbonitrile (100 mg, 337 μmol, Intermediate 524) and cyclopropyl(1H-imidazol-2-yl)methanone (55.1 mg, 405 μmol) to give 35.0 mg (91% purity, 23% yield) of the title compound.

LC-MS (Method 1): Rₜ=0.81 min; MS (ESIpos): m/z=417 [M+H]⁺

¹H NMR (400 MHz, CHLOROFORM-d) δ ppm: 8.65 and 8.63 (2d, 1H), 8.10 and 8.09 (2d, 1H), 7.00 (2s, 2H), 6.27 6.26 (2s, 1H), 5.24 (s, 2H), 4.14-4.22 (m, 2H), 4.06-4.14 (m, 2H), 3.46 (d, 1H, one diastereomer), 3.25-3.32 (m, 1H, one diastereomer), 2.91-3.03 (m, 1H), 2.65-2.90 (m, 3H), 2.20-2.43 (m, 2H), 1.19-1.37 (m, 1H), 1.05-1.18 (m, 1H), 0.78-0.91 (m, 1H), 0.68-0.77 (m, 1H), 0.42-0.52 (m, 2H), 0.33-0.41 (m, 1H).

### Example 710

### 2-amino-5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}nicotinonitrile (Diastereomer 1)

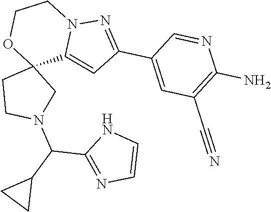 [SMILES: N#Cc1cc(-c2cc3n(n2)CCO[C@@]32CCN(C(c3ncc[nH]3)C3CC3)C2)cnc1N]

The title compound from Example 709 was separated into diastereomers by preparative chiral HPLC to give 8.70 mg (99% purity) of the title compound (diastereomer 1 Rₜ=6.0-7.5 min).

Preparative Chiral HPLC Method:

Instrument: Sepiatec: Prep SFC100; Column: Chiral Art Cellulose SA 10μ, 480×30 mm; eluent A: CO2; eluent B: 2-propanol+0.4 vol % diethylamine; isocratic: 30% B; flow: 100 mL/min; temperature: 40° C.; BPR: 150 bar; UV: 272 nm

Analytical Chiral HPLC Method:

Instrument: Waters Acquity UPC2 QDA; Column: Chiral Art Cellulose SA 3μ, 100×4.6; eluent A: CO2; eluent B: 2-propanol+0.4 vol % diethylamine; isocratic: 30% B; flow: 4 mL/min; temperature: 40° C.; BPR: 1800 psi UV: 254 nm

Analytical chiral HPLC: Rₜ=1.58 min

LC-MS (Method 1): Rₜ=0.81 min; MS (ESIpos): m/z=417 [M+H]⁺

¹H NMR (CHLOROFORM-d, 400 MHz): δ (ppm) 9.47 (br s, 1H), 8.63 (d, 1H), 8.09 (d, 1H), 6.95-7.06 (m, 2H), 6.26 (s, 1H), 5.23 (s, 2H), 4.02-4.20 (m, 4H), 3.25-3.32 (m, 1H), 2.91-3.04 (m, 2H), 2.81 (d, 1H), 2.66-2.74 (m, 1H), 2.38 (dt, 1H), 2.21-2.29 (m, 1H), 1.09-1.18 (m, 1H), 0.69-0.78 (m, 1H), 0.43-0.52 (m, 2H), 0.33-0.41 (m, 1H).

### Example 711

### 2-amino-5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}nicotinonitrile (Diastereomer 2)

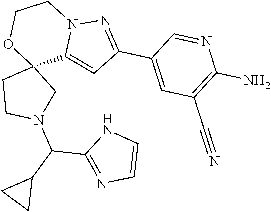 [SMILES: N#Cc1cc(-c2cc3n(n2)CCO[C@@]32CCN(C(c3ncc[nH]3)C3CC3)C2)cnc1N]

For the preparation of the diastereomeric mixture of the title compound see Example 709. The diastereomers were separated by preparative chiral HPLC (method see Example 710) to give 10.7 mg (99% purity) of the title compound (diastereomer 2 Rₜ=13.0-17.0 min).

Analytical chiral HPLC (method see Example 710): Rₜ=3.57 min

LC-MS (Method 1): Rₜ=0.81 min; MS (ESIpos): m/z=417 [M+H]⁺

¹H NMR (CHLOROFORM-d, 400 MHz): δ (ppm) 9.57 (br s, 1H), 8.65 (d, 1H), 8.10 (d, 1H), 7.00 (br s, 2H), 6.27 (s, 1H), 5.26 (s, 2H), 4.17-4.21 (m, 2H), 4.09-4.14 (m, 2H), 3.46 (d, 1H), 2.86 (t, 2H), 2.79 (dd, 2H), 2.30-2.39 (m, 1H), 2.19-2.28 (m, 1H), 1.07-1.17 (m, 1H), 0.68-0.75 (m, 1H), 0.42-0.51 (m, 2H), 0.34-0.41 (m, 1H).

### Example 712

### 5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}-3-(difluoromethoxy)pyridin-2-amine (Diastereomer 1 and Diastereomer 2)

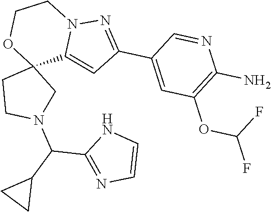 [SMILES: Nc1ncc(-c2cc3n(n2)CCO[C@@]32CCN(C(c3ncc[nH]3)C3CC3)C2)cc1OC(F)F]

The compound was prepared similarly to Example 706 using 3-(difluoromethoxy)-5-[(3′R)-6,7-dihydrospiro[pyrazolo[5,1-c][1,4]oxazine-4,3′-pyrrolidin]-2-yl]pyridin-2-amine (100 mg, 296 μmol, Intermediate 525) and cyclopropyl(1H-imidazol-2-yl)methanone (48.4 mg, 356 μmol) to give 39.0 mg (96% purity, 28% yield) of the title compound.

LC-MS (Method 1): Rₜ=0.87 min; MS (ESIpos): m/z=458 [M+H]⁺

¹H NMR (400 MHz, CHLOROFORM-d): 6 ppm 0.009 (16.00), 0.018 (0.63), 0.317 (0.46), 0.389 (0.42), 0.396 (0.58), 0.407 (0.82), 0.424 (0.69), 0.435 (0.47), 0.654 (0.69), 0.672 (0.67), 0.686 (0.49), 0.769 (1.22), 0.783 (1.28), 0.793 (1.25), 0.803 (1.19), 0.821 (1.97), 0.828 (1.08), 1.042 (0.77), 1.194 (15.25), 1.219 (1.82), 1.226 (2.26), 1.277 (1.89), 1.312 (0.89), 1.363 (1.73), 1.556 (2.75), 1.988 (0.83), 2.181 (0.40), 2.637 (0.69), 2.655 (0.66), 2.750 (0.49), 2.775 (0.43), 3.381 (0.58), 3.409 (0.53), 4.014 (0.53), 4.028 (0.62), 4.041 (0.65), 4.054 (1.13), 4.064 (0.81), 4.095 (0.58), 4.107 (0.89), 4.118 (0.94), 4.128 (0.86), 4.134 (0.79), 4.727 (1.59), 5.242 (3.02), 6.202 (1.09), 6.214 (1.78), 6.312 (0.56), 6.317 (0.77), 6.495 (1.08), 6.500 (1.54), 6.678 (0.55), 6.683 (0.67), 6.936 (2.32), 7.631 (0.64), 7.646 (0.87), 8.228 (0.97), 8.233 (1.01), 8.245 (1.43), 8.250 (1.33).

### Example 713

### 5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}-3-(difluoromethoxy)pyridin-2-amine (Diastereomer 1)

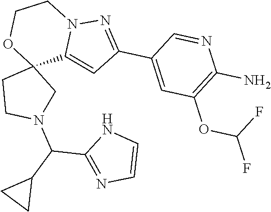 [SMILES: Nc1ncc(-c2cc3n(n2)CCO[C@@]32CCN(C(c3ncc[nH]3)C3CC3)C2)cc1OC(F)F]

The compound from Example 712 was separated into diastereomers by preparative chiral HPLC to give 8.80 mg (99% purity) of the title compound (diastereomer 1 Rₜ=4.5-7.5 min).

Preparative Chiral HPLC Method:

Instrument: Sepiatec: Prep SFC100; Column: Chiralpak ID 5μ, 250×50; eluent A: CO2; eluent B: 2-propanol+0.4 vol % diethylamine; isocratic: 40% B; flow: 100 mL/min; temperature: 40° C.; BPR: 150 bar; UV: 267 nm

Analytical Chiral HPLC Method:

Instrument: Waters Acquity UPC2 QDA; Column: Chiralpak ID 5μ, 100×4.6; eluent A: CO2; eluent B: 2-propanol+0.4 vol % diethylamine; isocratic: 40% B; flow: 4 mL/min; temperature: 40° C.; BPR: 100 bar; UV: 267 nm

Analytical chiral HPLC Rₜ=1.40 min

LC-MS (Method 1): Rₜ=0.87 min; MS (ESIpos): m/z=458 [M+H]⁺

¹H-NMR (400 MHz, CHLOROFORM-d) δ [ppm]: 0.277 (1.23), 0.293 (2.46), 0.304 (3.18), 0.322 (2.11), 0.338 (0.58), 0.395 (5.42), 0.409 (6.29), 0.424 (4.69), 0.429 (3.26), 0.444 (1.08), 0.628 (0.63), 0.644 (1.86), 0.657 (2.81), 0.673 (2.56), 0.689 (1.18), 0.794 (0.60), 0.811 (1.30), 0.829 (0.70), 1.049 (1.58), 1.066 (2.06), 1.101 (1.66), 1.117 (1.20), 1.136 (2.53), 1.151 (2.76), 1.188 (5.79), 1.215 (1.48), 1.601 (5.42), 1.958 (0.43), 2.111 (1.61), 2.134 (0.40), 2.156 (1.43), 2.171 (1.76), 2.177 (1.78), 2.191 (3.39), 2.205 (2.68), 2.211 (2.63), 2.225 (2.18), 2.277 (1.78), 2.295 (3.44), 2.312 (2.98), 2.329 (2.01), 2.347 (1.15), 2.549 (11.64), 2.567 (0.83), 2.602 (1.33), 2.642 (2.61), 2.663 (1.20), 2.738 (3.69), 2.761 (3.51), 2.866 (2.31), 2.893 (5.42), 2.925 (5.77), 2.953 (2.33), 3.191 (1.55), 3.211 (2.53), 3.229 (2.33), 3.248 (1.25), 3.960 (0.63), 3.975 (1.08), 3.990 (2.83), 4.004 (7.37), 4.018 (7.87), 4.030 (4.59), 4.047 (1.23), 4.059 (1.73), 4.085 (7.55), 4.097 (12.11), 4.110 (4.36), 4.713 (12.89), 6.193 (16.00), 6.302 (7.22), 6.485 (13.84), 6.669 (6.72), 6.917 (5.09), 6.929 (3.96), 6.952 (5.24), 7.451 (1.58), 7.622 (9.71), 8.219 (8.15), 8.223 (8.03), 9.379 (1.23).

### Example 714

### 5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}-3-(difluoromethoxy)pyridin-2-amine (Diastereomer 2)

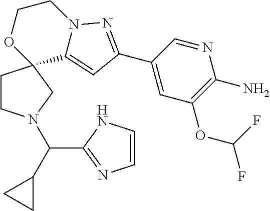 [SMILES: Nc1ncc(-c2cc3n(n2)CCO[C@@]32CCN(C(c3ncc[nH]3)C3CC3)C2)cc1OC(F)F]

The diastereomers were separated by preparative chiral HPLC (method see Example 713) to give 9.70 mg (99% purity) of the title compound (diastereomer 2 Rₜ=9.3-13.3 min).

Analytical chiral HPLC (method see Example 713): Rₜ=2.82 min

LC-MS (Method 1): Rₜ=0.87 min; MS (ESIpos): m/z=458 [M+H]⁺

¹H-NMR (400 MHz, CHLOROFORM-d) δ [ppm]: 0.304 (2.39), 0.317 (2.86), 0.335 (1.89), 0.360 (1.43), 0.379 (4.87), 0.395 (6.24), 0.409 (3.73), 0.624 (1.35), 0.638 (2.79), 0.657 (2.73), 0.671 (1.30), 0.811 (0.68), 0.828 (0.44), 1.006 (0.54), 1.037 (2.00), 1.049 (2.03), 1.059 (1.98), 1.068 (1.35), 1.093 (0.54), 1.104 (0.90), 1.120 (0.90), 1.138 (3.08), 1.153 (3.16), 1.189 (4.56), 1.736 (3.10), 2.112 (1.50), 2.143 (0.88), 2.160 (1.81), 2.177 (2.76), 2.195 (3.48), 2.212 (2.02), 2.230 (1.74), 2.247 (3.54), 2.264 (2.55), 2.281 (1.70), 2.298 (0.86), 2.550 (1.60), 2.568 (0.76), 2.698 (4.94), 2.725 (6.23), 2.732 (5.13), 2.756 (4.64), 2.771 (5.05), 2.788 (8.31), 2.806 (3.83), 3.371 (4.73), 3.397 (4.25), 4.028 (3.83), 4.040 (8.80), 4.051 (7.15), 4.077 (0.85), 4.108 (6.68), 4.118 (7.88), 4.122 (7.62), 4.135 (3.15), 4.769 (11.95), 6.195 (16.00), 6.312 (5.68), 6.494 (11.56), 6.678 (5.38), 6.920 (6.52), 6.936 (6.55), 7.453 (0.48), 7.636 (9.39), 8.235 (5.80), 9.556 (1.43).

### Example 715

### 5-{1-[1-(1H-imidazol-2-yl)propyl]-5′,6′-dihydrospiro[azetidine-3,4′-pyrrolo[1,2-b]pyrazol]-2′-yl}-3-(trifluoromethyl)pyridin-2-amine (Enantiomer 1 and Enantiomer 2)

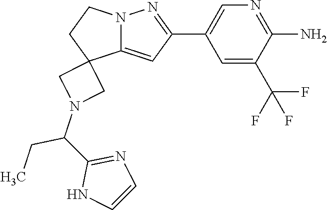 [SMILES: CCC(c1ncc[nH]1)N1CC2(CCn3nc(-c4cnc(N)c(C(F)(F)F)c4)cc32)C1]

5-(5′,6′-Dihydrospiro[azetidine-3,4′-pyrrolo[1,2-b]pyrazol]-2′-yl)-3-(trifluoromethyl)pyridin-2-amine (210 mg, 679 μmol, Intermediate 526) and 1-(1H-imidazol-2-yl)propan-1-one hydrogen chloride (1/1) (109 mg, 679 μmol) were dissolved in methanol (2.3 mL). N,N-Diisopropylethylamine (590 μL, 3.4 mmol) and titanium(IV) isopropoxide (600 μL, 2.0 mmol; 1) were added and the mixture was stirred for 16 h at 60° C. The suspension was allowed to cool down, sodium cyanoborohydride (128 mg, 2.04 mmol) was added and the mixture was stirred for 3 h at 60° C. The reaction mixture was allowed to cool down, diluted with water (1 mL) and stirred for 30 min. The suspension was diluted with methanol and filtered over celite. The filtrate was concentrated. The crude product was purified by column chromatography (silica gel, dichloromethane/methanol (0-10%) gradient). The combined fractions were dissolved in dichloromethane/methanol and washed two times with saturated sodium hydrogen carbonate solution. The organic phase was dried over a hydrophobic filter and concentrated to give 70.0 mg (100% purity, 25% yield) of the title compound.

LC-MS (Method 1): Rₜ=0.90 min; MS (ESIpos): m/z=418 [M+H]⁺

¹H-NMR (400 MHz, DMSO-d6) δ [ppm]: 0.671 (4.42), 0.690 (10.55), 0.708 (4.73), 1.233 (1.09), 1.572 (0.54), 1.586 (1.03), 1.609 (1.29), 1.627 (1.26), 1.634 (1.21), 1.641 (1.04), 1.653 (1.14), 1.668 (0.49), 2.336 (0.77), 2.518 (9.22), 2.523 (6.63), 2.678 (0.80), 2.728 (13.50), 2.752 (2.19), 2.767 (3.44), 2.772 (3.46), 2.787 (2.44), 2.889 (16.00), 3.189 (1.88), 3.206 (2.29), 3.233 (2.60), 3.250 (2.85), 3.316 (3.76), 3.345 (2.22), 3.356 (1.71), 3.368 (1.48), 3.478 (2.26), 3.495 (1.99), 4.059 (2.80), 4.077 (5.30), 4.095 (2.76), 5.759 (6.17), 6.537 (6.92), 6.584 (13.63), 6.807 (1.09), 7.036 (1.08), 7.951 (2.00), 8.007 (4.00), 8.012 (4.03), 8.588 (3.69), 8.593 (3.67), 11.789 (1.78).

### Example 716

### 5-{1-[1-(1H-imidazol-2-yl)propyl]-5′,6′-dihydrospiro[azetidine-3,4′-pyrrolo[1,2-b]pyrazol]-2′-yl}-3-(trifluoromethyl)pyridin-2-amine (Enantiomer 1)

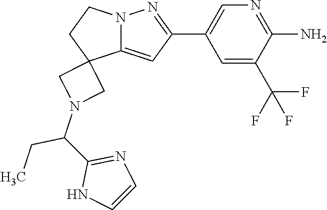 [SMILES: CCC(c1ncc[nH]1)N1CC2(CCn3nc(-c4cnc(N)c(C(F)(F)F)c4)cc32)C1]

The title compound from Example 715 was separated into enantiomers by preparative chiral HPLC to give 17.0 mg (100% purity) of the title compound (enantiomer 1 Rₜ=4.6-6.4 min).

Preparative Chiral HPLC Method:

Instrument: Sepiatec: Prep SFC100; Column: Chiral Art Cellulose-SC 5p, 250×50; eluent A: CO2; eluent B: 2-propanol+0.4 vol % diethylamine; isocratic: 30% B; flow: 100 mL/min; temperature: 40° C.; BPR: 150 bar; UV: 271 nm

Analytical Chiral HPLC Method:

Instrument: Waters Acquity UPC2 QDA; Column: Chiral Art Cellulose-SC 3μ, 100×4.6; eluent A: CO2; eluent B: 2-propanol+0.4 vol % diethylamine; isocratic: 30% B; flow: 4 mL/min; temperature: 40° C.; BPR: 1800 psi; UV: 280 nm

Analytical chiral HPLC: Rₜ=1.14 min

[α]²⁰_{D}: +34.7° (c=1.00, methanol)

LC-MS (Method 1): Rₜ=0.90 min; MS (ESIpos): m/z=418 [M+H]⁺

¹H-NMR (400 MHz, CHLOROFORM-d) δ [ppm]: 0.737 (3.78), 0.756 (8.55), 0.774 (4.16), 1.137 (0.46), 1.153 (0.47), 1.189 (0.98), 1.396 (0.46), 1.460 (0.49), 1.479 (0.64), 1.482 (0.62), 1.495 (0.76), 1.516 (0.80), 1.535 (0.63), 1.693 (0.70), 1.702 (0.76), 1.711 (0.76), 1.721 (0.82), 1.726 (0.71), 1.736 (0.65), 1.745 (0.59), 1.755 (0.53), 2.769 (1.48), 2.773 (1.48), 2.785 (2.78), 2.791 (2.72), 2.807 (1.73), 3.275 (1.65), 3.294 (1.94), 3.361 (1.78), 3.379 (2.57), 3.442 (2.11), 3.460 (1.51), 3.472 (2.50), 3.478 (1.70), 3.489 (2.82), 3.501 (1.42), 3.510 (1.20), 4.091 (2.91), 4.108 (5.27), 4.125 (2.74), 4.961 (4.06), 6.332 (8.12), 6.957 (16.00), 8.047 (2.68), 8.052 (2.74), 8.550 (2.62), 8.554 (2.60).

### Example 717

### 5-{1-[1-(1H-imidazol-2-yl)propyl]-5′,6′-dihydrospiro[azetidine-3,4′-pyrrolo[1,2-b]pyrazol]-2′-yl}-3-(trifluoromethyl)pyridin-2-amine (Enantiomer 2)

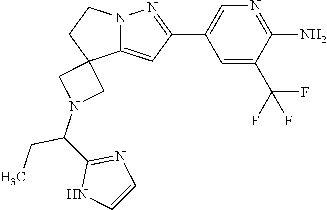 [SMILES: CCC(c1ncc[nH]1)N1CC2(CCn3nc(-c4cnc(N)c(C(F)(F)F)c4)cc32)C1]

For the preparation of the enantiomers of the title compound see Example 715. The enantiomers were separated by preparative chiral HPLC (method see Example 716) to give 14.0 mg (100% purity) of the title compound (enantiomer 2 Rₜ=11.0-14.0 min).

Analytical chiral HPLC (method see Example 716): Rₜ=2.88 min

[α]²⁰_{D}: −34.4° (c=1.00, methanol)

LC-MS (Method 1): Rₜ=0.90 min; MS (ESIpos): m/z=418 [M+H]⁺

¹H-NMR (400 MHz, CHLOROFORM-d) δ [ppm]: 0.737 (4.55), 0.756 (10.26), 0.774 (4.99), 1.137 (0.81), 1.153 (0.86), 1.189 (1.40), 1.396 (0.44), 1.454 (0.55), 1.472 (0.74), 1.488 (0.87), 1.510 (0.94), 1.529 (0.76), 1.673 (0.59), 1.682 (0.58), 1.691 (0.99), 1.701 (1.02), 1.710 (1.00), 1.719 (1.03), 1.725 (0.91), 1.734 (0.80), 1.743 (0.71), 1.754 (0.65), 1.942 (0.76), 2.769 (1.80), 2.773 (1.78), 2.786 (3.32), 2.792 (3.15), 2.807 (2.08), 3.271 (1.85), 3.289 (2.15), 3.357 (1.92), 3.374 (2.82), 3.435 (2.31), 3.453 (1.70), 3.465 (3.03), 3.483 (2.61), 3.491 (1.63), 3.501 (1.30), 4.091 (3.56), 4.109 (6.29), 4.127 (3.26), 4.951 (4.72), 6.332 (8.90), 6.955 (16.00), 8.049 (3.16), 8.053 (3.21), 8.552 (3.07), 8.556 (3.04).

### Example 718

### 5-{(3′R)-1′-[cyclobutyl(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine (Diastereomer 1 and Diastereomer 2)

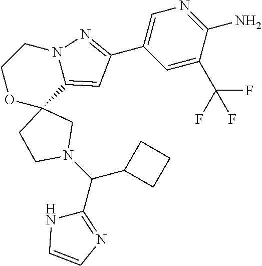 [SMILES: Nc1ncc(-c2cc3n(n2)CCO[C@@]32CCN(C(c3ncc[nH]3)C3CCC3)C2)cc1C(F)(F)F]

5-[(3′R)-6,7-Dihydrospiro[pyrazolo[5,1-c][1,4]oxazine-4,3′-pyrrolidin]-2-yl]-3-(trifluoromethyl)pyridin-2-amine hydrogen chloride (1/1) (107 mg, 284 μmol) and cyclobutyl(1H-imidazol-2-yl)methanone (64.0 mg, 426 μmol, Intermediate 563) were dissolved in methanol (6.1 mL). N,N-Diisopropylethylamine (200 μL, 1.1 mmol) and titanium(IV) isopropoxide (250 μL, 850 μmol) were added and the mixture was stirred for 3 h at 60° C. Sodium cyanoborohydride (53.6 mg, 852 μmol) was added and the mixture was stirred over night at 60° C. The cold reaction mixture was filtered. The filtrate was diluted with ethyl acetate. The organic phase was washed with a saturated sodium bicarbonate solution, water and brine, dried and concentrated. The crude product was purified by column chromatography (silica gel NH2, hexane/ethyl acetate (0-100%) gradient). The combined fractions were purified by preparative HPLC to give 40.0 mg (97% purity, 29% yield) of the title compound.

LC-MS (Method 1): Rₜ=1.00 min; MS (ESIpos): m/z=475 [M+H]⁺

¹H-NMR (400 MHz, DMSO-d6) δ [ppm]: 0.851 (0.51), 1.231 (1.39), 1.465 (0.44), 1.487 (1.47), 1.512 (2.62), 1.536 (2.52), 1.559 (1.07), 1.593 (1.66), 1.604 (1.75), 1.613 (1.85), 1.621 (1.81), 1.657 (1.90), 1.678 (1.99), 1.700 (0.90), 1.742 (1.06), 1.764 (1.71), 1.790 (1.55), 1.812 (0.89), 1.839 (1.19), 1.861 (2.08), 1.886 (2.64), 1.909 (2.01), 1.931 (0.60), 1.976 (0.49), 1.987 (0.85), 1.993 (1.06), 2.011 (1.71), 2.026 (2.66), 2.045 (3.61), 2.064 (3.97), 2.083 (4.20), 2.097 (2.92), 2.112 (2.47), 2.130 (1.40), 2.331 (2.18), 2.336 (1.00), 2.518 (12.26), 2.523 (8.55), 2.576 (2.87), 2.595 (1.58), 2.617 (0.55), 2.673 (2.33), 2.678 (1.22), 2.687 (3.70), 2.713 (4.10), 2.737 (1.04), 2.758 (3.68), 2.769 (2.75), 2.783 (3.85), 2.808 (0.63), 2.820 (1.06), 2.842 (2.24), 2.851 (3.69), 2.863 (2.51), 2.877 (2.58), 2.884 (1.89), 2.905 (0.73), 2.937 (3.90), 2.962 (3.25), 3.535 (6.06), 3.559 (5.62), 3.920 (0.41), 3.936 (1.01), 3.949 (2.50), 3.961 (3.94), 3.976 (5.23), 3.989 (4.17), 4.046 (6.68), 4.054 (5.92), 5.759 (5.17), 6.437 (10.76), 6.483 (16.00), 6.576 (12.83), 6.796 (1.37), 7.014 (1.21), 8.005 (8.64), 8.010 (8.66), 8.585 (8.08), 8.589 (7.92), 11.785 (3.61).

### Example 719

### 5-{(3′R)-1′-[cyclobutyl(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine (Diastereomer 1)

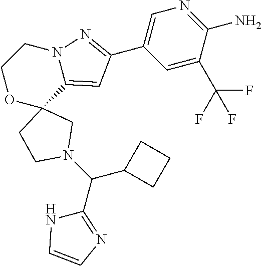 [SMILES: Nc1ncc(-c2cc3n(n2)CCO[C@@]32CCN(C(c3ncc[nH]3)C3CCC3)C2)cc1C(F)(F)F]

The title compound from Example 718 was separated into diastereomers by preparative chiral HPLC (diastereomer 1 Rₜ=7.4-8.8 min).

Preparative Chiral HPLC Method:

Instrument: Sepiatec: Prep SFC100; Column: Chiral Art Aylose-SA 5μ, 250×50; eluent A: CO2; eluent B: methanol+0.2 vol % aqueous ammonia (32%); isocratic: 15% B; flow: 100 mL/min; temperature: 40° C.; BPR: 150 bar; UV: 267 nm

Analytical Chiral HPLC Method:

Instrument: Waters Acquity UPC2 QDA; Column: Chiral Art Amylose-SA 3μ, 100×4.6; eluent A: CO2; eluent B: methanol+0.2 vol % aqueous ammonia (32%); isocratic: 20% B; flow: 4 mL/min; temperature: 40° C.; BPR: 100 bar; UV: 267 nm

Analytical chiral HPLC: Rₜ=1.29 min The isolated fraction was purified by preparative HPLC once more to give 6.30 mg (95% purity) of the title compound.

LC-MS (Method 1): Rₜ=1.00 min; MS (ESIpos): m/z=474 [M+H]⁺

¹H-NMR (400 MHz, METHANOL-d4) 5 [ppm]: 0.097 (0.60), 0.881 (0.62), 0.899 (1.26), 0.916 (0.59), 1.286 (3.78), 1.331 (1.54), 1.396 (0.46), 1.587 (0.41), 1.613 (1.13), 1.637 (2.94), 1.658 (5.37), 1.679 (3.44), 1.721 (1.55), 1.745 (1.67), 1.766 (0.80), 1.821 (0.44), 1.844 (1.30), 1.866 (2.21), 1.890 (2.01), 1.911 (1.09), 1.935 (1.27), 1.958 (1.88), 1.984 (2.54), 2.007 (1.96), 2.030 (0.91), 2.147 (0.54), 2.166 (1.50), 2.181 (2.87), 2.200 (6.82), 2.218 (5.84), 2.234 (3.11), 2.249 (0.98), 2.268 (0.50), 2.655 (1.27), 2.672 (2.80), 2.694 (3.21), 2.713 (1.28), 2.774 (5.21), 2.800 (6.23), 2.827 (1.46), 2.841 (2.19), 2.848 (2.41), 2.862 (3.48), 2.883 (3.27), 2.906 (2.26), 2.927 (1.16), 3.014 (5.68), 3.040 (4.63), 3.130 (0.45), 3.478 (0.50), 3.574 (6.22), 3.599 (5.75), 4.062 (1.56), 4.071 (2.80), 4.082 (5.22), 4.092 (7.97), 4.099 (4.22), 4.107 (8.70), 4.111 (8.22), 4.855 (1.01), 5.491 (5.00), 6.466 (16.00), 6.979 (9.72), 8.124 (6.45), 8.129 (6.55), 8.538 (6.95), 8.542 (6.30).

### Example 720

### 5-{(3′R)-1′-[cyclobutyl(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine (Diastereomer 2)

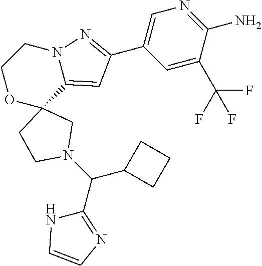 [SMILES: Nc1ncc(-c2cc3n(n2)CCO[C@@]32CCN(C(c3ncc[nH]3)C3CCC3)C2)cc1C(F)(F)F]

For the preparation of the diastereomeric mixture of the title compound see Example 718. The diastereomers were separated by preparative chiral HPLC (method see Example 719) (diastereomer 2 Rₜ=11.0-14.0 min).

Analytical chiral HPLC (method see Example 719): Rₜ=2.88 min The crude fraction was purified by preparative HPLC once more to give 2.60 mg (95% purity) of the title compound.

LC-MS (Method 1): Rₜ=1.00 min; MS (ESIpos): m/z=474 [M+H]⁺

¹H-NMR (400 MHz, METHANOL-d4) 5 [ppm]: 0.886 (1.34), 0.903 (2.00), 1.029 (0.48), 1.291 (7.58), 1.334 (2.41), 1.401 (0.79), 1.594 (1.59), 1.620 (2.97), 1.644 (3.21), 1.667 (2.78), 1.677 (2.34), 1.686 (2.04), 1.705 (2.14), 1.730 (2.24), 1.757 (2.13), 1.778 (1.02), 1.830 (0.47), 1.854 (1.41), 1.875 (2.36), 1.900 (2.16), 1.921 (1.18), 1.946 (1.36), 1.967 (2.07), 1.993 (2.76), 2.016 (2.22), 2.040 (1.06), 2.117 (0.83), 2.136 (1.62), 2.151 (2.28), 2.169 (3.40), 2.188 (2.88), 2.210 (3.44), 2.223 (4.13), 2.242 (3.55), 2.256 (1.57), 2.275 (0.88), 2.647 (1.22), 2.660 (2.02), 2.668 (2.55), 2.682 (2.65), 2.702 (1.39), 2.829 (1.59), 2.855 (5.59), 2.870 (3.29), 2.881 (6.78), 2.918 (2.41), 2.940 (2.41), 2.962 (1.40), 2.974 (6.97), 3.000 (4.64), 3.133 (0.72), 3.482 (0.69), 3.596 (6.32), 3.620 (5.87), 4.017 (0.97), 4.032 (1.95), 4.048 (4.02), 4.055 (3.47), 4.064 (5.02), 4.075 (4.00), 4.100 (11.85), 4.110 (6.67), 4.614 (0.58), 5.345 (0.41), 5.495 (2.86), 6.451 (16.00), 7.005 (12.49), 8.128 (6.99), 8.132 (7.06), 8.545 (7.08).

### Example 721

5-{(3′R)-1′-[1-(1H-imidazol-2-yl)-2-methylpropyl]-6,7-dihydrospiro[pyrazolo[5,1-c][1,4]oxazine-4,3′-pyrrolidin]-2-yl}-3-(trifluoromethyl)pyridin-2-amine (Diastereomer 1 and Diastereomer 2)

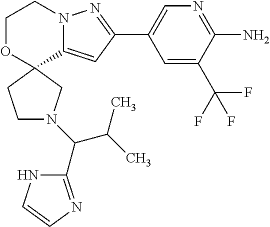 [SMILES: CC(C)C(c1ncc[nH]1)N1CC[C@]2(C1)OCCn1nc(-c3cnc(N)c(C(F)(F)F)c3)cc12]

5-[(3′R)-6,7-Dihydrospiro[pyrazolo[5,1-c][1,4]oxazine-4,3′-pyrrolidin]-2-yl]-3-(trifluoromethyl)pyridin-2-amine hydrogen chloride (1/1) (277 mg, 738 μmol) and 1-(1H-imidazol-2-yl)-2-methylpropan-1-one (153 mg, 1.11 mmol, Intermediate 542) were dissolved in methanol (8.0 mL). N,N-Diisopropylethylamine (510 μL, 3.0 mmol) and titanium(IV) isopropoxide (650 μL, 2.2 mmol) were added and the mixture was stirred for 3 h at 60° C. To the cold reaction mixture was added sodium cyanoborohydride (139 mg, 2.21 mmol) and the mixture was stirred for 16 h at 60° C. Titanium(IV) isopropoxide (433 μL, 1.46 mmol) and sodium cyanoborohydride (92 mg, 1.47 mmol) were added again and stirred for 16 h at 60° C. The reaction mixture was allowed to cool down, diluted with water and stirred for 15 min. The suspension was filtered and the filter cake was washed with methanol. The filtrate was concentrated. The crude product was purified by column chromatography (silica gel NH2, hexane/ethyl acetate (0-100%) and ethyl acetate/ethanol (0-40%) gradient). The combined fractions were diluted with ethyl acetate. The organic phase was washed 3 times with a sodium bicarbonate solution and concentrated. The residue was purified by preparative HPLC to give 15.0 mg (95% purity, 4% yield) of the title compound.

LC-MS (Method 1): Rₜ=0.99 min; MS (ESIpos): m/z=462 [M+H]⁺

¹H-NMR (400 MHz, DMSO-d6) δ [ppm]: −0.045 (0.59), 0.694 (4.97), 0.701 (4.09), 0.711 (5.39), 0.717 (3.85), 0.929 (4.53), 0.945 (6.32), 0.960 (3.31), 1.232 (1.00), 1.988 (0.43), 2.005 (0.61), 2.022 (0.80), 2.037 (0.74), 2.055 (1.45), 2.074 (1.79), 2.084 (16.00), 2.097 (0.70), 2.114 (0.48), 2.131 (0.58), 2.149 (0.77), 2.169 (0.87), 2.186 (0.72), 2.518 (4.67), 2.523 (2.85), 2.577 (0.88), 2.598 (0.47), 2.608 (0.48), 2.622 (0.48), 2.750 (0.57), 2.772 (0.49), 2.789 (0.60), 2.801 (2.08), 2.825 (2.50), 2.934 (1.12), 2.960 (0.84), 2.991 (1.55), 3.017 (1.23), 3.159 (0.78), 3.171 (0.79), 3.353 (2.39), 3.364 (1.51), 3.375 (1.78), 3.975 (2.68), 3.987 (2.29), 4.046 (2.49), 4.059 (3.29), 4.070 (1.50), 5.759 (1.14), 6.375 (3.84), 6.427 (5.43), 6.576 (4.99), 6.846 (1.37), 7.037 (1.02), 7.061 (0.72), 8.002 (3.32), 8.007 (2.20), 8.579 (3.00), 11.740 (1.32).

### Example 722

### 5-{(3′R)-1′-[1-(1H-imidazol-2-yl)-2-methylpropyl]-6,7-dihydrospiro[pyrazolo[5,1-c][1,4]oxazine-4,3′-pyrrolidin]-2-yl}-3-(trifluoromethyl)pyridin-2-amine (Diastereomer 1)

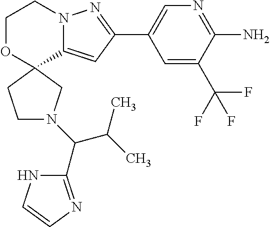 [SMILES: CC(C)C(c1ncc[nH]1)N1CC[C@]2(C1)OCCn1nc(-c3cnc(N)c(C(F)(F)F)c3)cc12]

The compound 5-{(3′R)-1′-[1-(1H-imidazol-2-yl)-2-methylpropyl]-6,7-dihydrospiro[pyrazolo[5,1-c][1,4]oxazine-4,3′-pyrrolidin]-2-yl}-3-(trifluoromethyl)pyridin-2-amine (diastereomer 1 and diastereomer 2, Example 721) was separated into diastereomers by preparative chiral HPLC to give 30.0 mg (95% purity, 8% yield) of the title compound (diastereomer 1 Rₜ=7.4-13.0 min).

Preparative Chiral HPLC Method:

Instrument: Sepiatec: Prep SFC100; Column: Chiral Art Amylose SA 5μ, 250×50; eluent A: CO2; eluent B: 2-propanol+0.4 vol % diethylamine; isocratic: 20% B; flow: 100 mL/min; temperature: 40° C.; BPR: 150 bar; UV: 267 nm

Analytical Chiral HPLC Method:

Instrument: Waters Acquity UPC2 QDA; Column: Chiral Art Amylose SA 3μ, 100×4.6; eluent A: CO2; eluent B: 2-propanol+0.4 vol % diethylamine; isocratic: 20% B; flow: 4 mL/min; temperature: 40° C.; BPR: 1800 psi; UV: 254 nm

Analytical chiral HPLC Rₜ=2.87 min

LC-MS (Method 1): Rₜ=0.99 min; MS (ESIpos): m/z=462 [M+H]⁺

¹H-NMR (400 MHz, DMSO-d6) δ [ppm]: 0.695 (2.60), 0.701 (2.15), 0.712 (2.77), 0.718 (2.00), 0.733 (4.24), 0.749 (4.19), 0.847 (4.51), 0.863 (4.68), 0.929 (2.39), 0.945 (3.20), 0.960 (1.70), 1.154 (4.00), 1.173 (7.77), 1.190 (4.11), 1.232 (0.67), 1.944 (0.42), 1.961 (0.67), 1.978 (0.77), 1.988 (16.00), 1.994 (0.60), 2.023 (0.42), 2.055 (0.73), 2.065 (0.56), 2.083 (0.59), 2.148 (0.44), 2.170 (0.46), 2.295 (0.44), 2.518 (2.15), 2.523 (1.46), 2.578 (0.40), 2.801 (1.06), 2.826 (1.26), 2.935 (0.56), 2.961 (0.43), 2.992 (0.78), 3.017 (0.61), 3.354 (1.10), 3.365 (0.74), 3.375 (0.92), 3.976 (1.31), 3.988 (1.13), 4.000 (1.34), 4.017 (3.60), 4.035 (3.70), 4.046 (1.33), 4.053 (2.16), 4.059 (1.72), 4.071 (0.82), 4.124 (0.43), 4.130 (0.44), 4.267 (0.80), 4.279 (0.88), 4.283 (0.86), 4.295 (0.77), 5.325 (1.90), 5.337 (1.86), 6.375 (2.14), 6.427 (3.29), 6.574 (2.51), 6.601 (0.42), 6.744 (0.60), 6.774 (0.66), 6.845 (0.85), 7.037 (0.64), 7.063 (0.43), 7.997 (0.82), 8.002 (1.70), 8.007 (1.26), 8.580 (1.68), 8.585 (1.24), 11.736 (0.73).

### Example 723

### 5-{(3′R)-1′-[1-(1H-imidazol-2-yl)-2-methylpropyl]-6,7-dihydrospiro[pyrazolo[5,1-c][1,4]oxazine-4,3′-pyrrolidin]-2-yl}-3-(trifluoromethyl)pyridin-2-amine (Diastereomer 2)

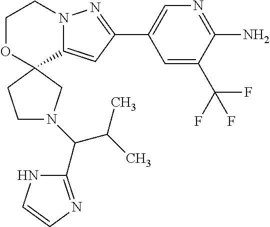 [SMILES: CC(C)C(c1ncc[nH]1)N1CC[C@]2(C1)OCCn1nc(-c3cnc(N)c(C(F)(F)F)c3)cc12]

The diastereomers were separated by preparative chiral HPLC (method see Example 722) to give 54.4 mg (95% purity, 15% yield) of the title compound (diastereomer 2 Rₜ=14.0-18.4 min).

Analytical chiral HPLC (method see Example 722): Rₜ=5.94 min

LC-MS (Method 1): Rₜ=0.99 min; MS (ESIpos): m/z=462 [M+H]⁺

¹H-NMR (400 MHz, DMSO-d6) δ [ppm]: 0.695 (2.60), 0.701 (2.15), 0.712 (2.77), 0.718 (2.00), 0.733 (4.24), 0.749 (4.19), 0.847 (4.51), 0.863 (4.68), 0.929 (2.39), 0.945 (3.20), 0.960 (1.70), 1.154 (4.00), 1.173 (7.77), 1.190 (4.11), 1.232 (0.67), 1.944 (0.42), 1.961 (0.67), 1.978 (0.77), 1.988 (16.00), 1.994 (0.60), 2.023 (0.42), 2.055 (0.73), 2.065 (0.56), 2.083 (0.59), 2.148 (0.44), 2.170 (0.46), 2.295 (0.44), 2.518 (2.15), 2.523 (1.46), 2.578 (0.40), 2.801 (1.06), 2.826 (1.26), 2.935 (0.56), 2.961 (0.43), 2.992 (0.78), 3.017 (0.61), 3.354 (1.10), 3.365 (0.74), 3.375 (0.92), 3.976 (1.31), 3.988 (1.13), 4.000 (1.34), 4.017 (3.60), 4.035 (3.70), 4.046 (1.33), 4.053 (2.16), 4.059 (1.72), 4.071 (0.82), 4.124 (0.43), 4.130 (0.44), 4.267 (0.80), 4.279 (0.88), 4.283 (0.86), 4.295 (0.77), 5.325 (1.90), 5.337 (1.86), 6.375 (2.14), 6.427 (3.29), 6.574 (2.51), 6.601 (0.42), 6.744 (0.60), 6.774 (0.66), 6.845 (0.85), 7.037 (0.64), 7.063 (0.43), 7.997 (0.82), 8.002 (1.70), 8.007 (1.26), 8.580 (1.68), 8.585 (1.24), 11.736 (0.73).

### Example 724

### 5-{(3R)-1-[cyclobutyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (Diastereomer 1 and Diastereomer 2)

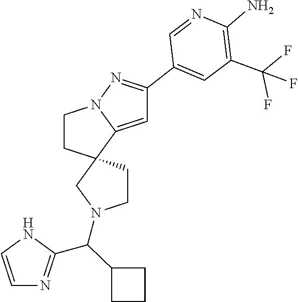 [SMILES: Nc1ncc(-c2cc3n(n2)CC[C@@]32CCN(C(c3ncc[nH]3)C3CCC3)C2)cc1C(F)(F)F]

5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (232 mg, 644 μmol) and cyclobutyl(1H-imidazol-2-yl)methanone (145 mg, 965 μmol, Intermediate 563) were dissolved in methanol (14 mL). N,N-Diisopropylethylamine (450 μL, 2.6 mmol) and titanium(IV) isopropoxide (570 μL, 1.9 mmol) were added and the mixture was stirred for 3 h at 60° C. The suspension was cooled to room temperature and sodium cyanoborohydride (121 mg, 1.93 mmol) was added and the mixture was stirred over night at 60° C. The cold reaction mixture was filtered. The filtrate was diluted with ethyl acetate, washed with saturated sodium bicarbonate solution, water and brine, dried and concentrated. The residue was purified by column chromatography (silica gel NH2, hexane/ethyl acetate (0-100%) and ethyl acetate/ethanol (0-100%) gradient). The combined fractions were purified by preparative HPLC to give 81.7 mg (90% purity, 25% yield) of the title compound.

LC-MS (Method 1): Rₜ=1.03 min; MS (ESIpos): m/z=458 [M+H]⁺

¹H-NMR (400 MHz, DMSO-d6) δ [ppm]: 1.154 (4.41), 1.172 (9.57), 1.190 (4.79), 1.232 (0.61), 1.475 (0.91), 1.499 (1.43), 1.523 (1.35), 1.546 (0.48), 1.628 (1.01), 1.636 (1.02), 1.647 (1.19), 1.655 (1.27), 1.675 (1.28), 1.695 (1.10), 1.723 (0.56), 1.748 (0.59), 1.756 (0.58), 1.777 (0.97), 1.799 (0.87), 1.853 (1.41), 1.876 (2.44), 1.893 (3.61), 1.912 (2.13), 1.936 (0.55), 1.987 (16.00), 2.106 (0.91), 2.379 (0.78), 2.394 (0.85), 2.401 (0.84), 2.411 (1.42), 2.421 (1.13), 2.428 (1.31), 2.437 (0.79), 2.443 (1.08), 2.461 (1.68), 2.477 (2.47), 2.518 (5.77), 2.523 (4.42), 2.563 (1.93), 2.580 (2.00), 2.602 (3.84), 2.608 (3.42), 2.631 (0.65), 2.702 (0.51), 2.723 (1.04), 2.743 (2.56), 2.766 (1.84), 2.784 (0.46), 2.800 (0.85), 2.823 (0.90), 2.838 (0.92), 2.857 (1.27), 2.879 (1.21), 2.901 (0.61), 3.159 (0.76), 3.172 (0.69), 3.557 (2.44), 3.582 (4.50), 3.608 (2.08), 3.999 (1.13), 4.017 (3.36), 4.035 (3.95), 4.053 (2.86), 4.057 (2.74), 4.062 (1.78), 4.074 (4.37), 4.092 (1.98), 5.758 (3.60), 6.240 (8.26), 6.294 (9.81), 6.515 (8.55), 6.803 (0.74), 7.019 (0.73), 7.980 (4.74), 8.559 (4.53), 11.734 (2.21).

### Example 725

### 5-{(3R)-1-[cyclobutyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (Diastereomer 1)

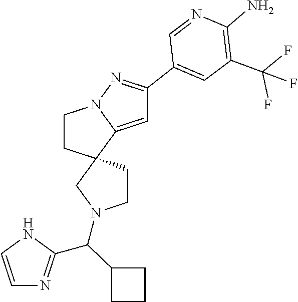 [SMILES: Nc1ncc(-c2cc3n(n2)CC[C@@]32CCN(C(c3ncc[nH]3)C3CCC3)C2)cc1C(F)(F)F]

The title compound from Example 724 was separated into diastereomers by preparative chiral HPLC (diastereomer 1 Rₜ=4.0-5.3 min).

Preparative Chiral HPLC Method:

Instrument: Sepiatec: Prep SFC100; Column: Chiralpak 5μ, 250×50; eluent A: CO2; eluent B: methanol+0.2 vol % aqueous ammonia (32%); isocratic: 30% B; flow: 100 mL/min; temperature: 40° C.; BPR: 150 bar; UV: 271 nm

Analytical Chiral HPLC Method:

Instrument: Waters Acquity UPC2 QDA; Column: Chiralpak IG 3μ, 100×4.6; eluent A: CO2; eluent B: methanol+0.2 vol % aqueous ammonia (32%); isocratic: 30% B; flow: 4 mL/min; temperature: 40° C.; BPR: 100 bar; UV: 280 nm

Analytical chiral HPLC: Rₜ=1.21 min

The isolated fraction was purified by preparative HPLC to give 8.10 mg (95% purity) of the title compound.

LC-MS (Method 1): Rₜ=1.04 min; MS (ESIpos): m/z=458 [M+H]⁺

¹H-NMR (400 MHz, DMSO-d6) δ [ppm]: 0.852 (0.51), 1.154 (0.93), 1.172 (1.81), 1.190 (0.88), 1.232 (1.68), 1.473 (1.03), 1.499 (1.50), 1.522 (1.42), 1.545 (0.52), 1.627 (1.14), 1.636 (1.12), 1.647 (1.42), 1.655 (1.37), 1.667 (1.34), 1.675 (1.44), 1.696 (1.26), 1.718 (0.64), 1.724 (0.56), 1.748 (0.96), 1.770 (1.50), 1.792 (1.21), 1.819 (0.93), 1.836 (1.08), 1.853 (2.52), 1.873 (2.71), 1.884 (2.07), 1.900 (2.76), 1.920 (1.67), 1.931 (0.74), 1.936 (0.68), 1.951 (0.53), 1.988 (3.43), 2.084 (7.92), 2.101 (0.99), 2.109 (0.89), 2.318 (0.70), 2.361 (0.60), 2.379 (1.49), 2.394 (1.51), 2.411 (2.38), 2.428 (1.18), 2.460 (1.70), 2.478 (4.93), 2.518 (8.80), 2.523 (7.21), 2.565 (1.76), 2.582 (1.79), 2.601 (0.78), 2.703 (0.88), 2.724 (1.92), 2.744 (4.67), 2.766 (3.25), 2.834 (0.79), 2.856 (1.29), 2.878 (1.32), 2.899 (0.71), 3.558 (4.38), 3.583 (4.04), 4.017 (0.75), 4.035 (0.79), 4.058 (3.42), 4.075 (7.01), 4.092 (3.33), 5.759 (4.25), 6.294 (16.00), 6.515 (8.65), 6.811 (0.60), 7.012 (0.63), 7.979 (4.92), 7.984 (4.93), 8.559 (4.55), 8.563 (4.44), 11.735 (1.80).

### Example 726

### 5-{(3R)-1-[cyclobutyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (Diastereomer 2)

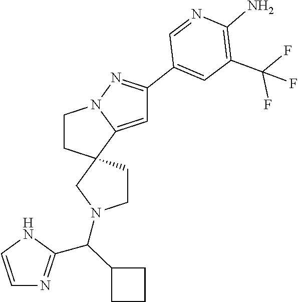 [SMILES: Nc1ncc(-c2cc3n(n2)CC[C@@]32CCN(C(c3ncc[nH]3)C3CCC3)C2)cc1C(F)(F)F]

For the preparation of the diastereomeric mixture of the title compound see Example 724. The diastereomers were separated by preparative chiral HPLC (method see Example 725) (diastereomer 2 Rₜ=7.3-9.7 min).

Analytical chiral HPLC (method see Example 725: Rₜ=2.81 min

The isolated fractions were purified by preparative HPLC again to give 8.60 mg (95% purity) of the title compound.

LC-MS (Method 1): Rₜ=1.04 min; MS (ESIpos): m/z=458 [M+H]⁺

¹H-NMR (400 MHz, DMSO-d6) δ [ppm]: 0.852 (0.49), 1.154 (3.13), 1.172 (6.02), 1.190 (2.89), 1.232 (1.56), 1.475 (0.95), 1.501 (1.43), 1.524 (1.31), 1.547 (0.48), 1.622 (0.65), 1.629 (1.08), 1.638 (1.01), 1.650 (1.21), 1.658 (1.17), 1.679 (1.31), 1.702 (1.16), 1.724 (0.56), 1.756 (0.86), 1.779 (1.38), 1.801 (1.12), 1.827 (0.66), 1.845 (0.81), 1.877 (2.56), 1.893 (5.91), 1.912 (3.53), 1.935 (0.41), 1.988 (11.46), 2.088 (0.45), 2.106 (1.04), 2.113 (0.96), 2.125 (0.91), 2.133 (0.84), 2.318 (0.69), 2.389 (0.71), 2.402 (1.43), 2.421 (2.11), 2.428 (1.57), 2.437 (1.69), 2.444 (2.27), 2.447 (2.03), 2.462 (2.21), 2.518 (8.62), 2.523 (6.27), 2.563 (2.03), 2.580 (2.14), 2.585 (2.24), 2.602 (6.56), 2.608 (6.34), 2.631 (1.02), 2.660 (0.77), 2.785 (0.76), 2.801 (1.57), 2.818 (1.28), 2.824 (1.60), 2.839 (1.26), 2.859 (1.21), 2.883 (1.23), 2.903 (0.67), 3.584 (3.96), 3.609 (3.62), 4.000 (0.88), 4.017 (2.65), 4.029 (0.64), 4.035 (3.76), 4.053 (3.47), 4.063 (2.44), 4.068 (2.36), 4.083 (1.64), 4.090 (0.55), 4.095 (0.49), 5.759 (2.14), 6.241 (16.00), 6.516 (8.11), 7.976 (4.63), 7.981 (4.65), 8.555 (4.27), 8.559 (4.21), 11.749 (1.19).

### Example 727

### 5-{(3R)-1-[1-(1H-imidazol-2-yl)-2,2-dimethylpropyl]-5′,6′-dihydrospiro[pyrrolidine-3,4′-pyrrolo[1,2-b]pyrazol]-2′-yl}-3-(trifluoromethyl)pyridin-2-amine (Diastereomer 1 and Diastereomer 2)

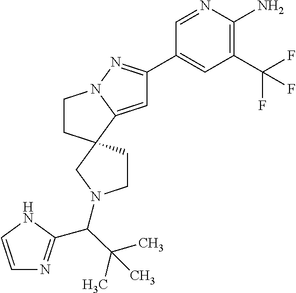 [SMILES: CC(C)(C)C(c1ncc[nH]1)N1CC[C@@]2(CCn3nc(-c4cnc(N)c(C(F)(F)F)c4)cc32)C1]

5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (50.0 mg, 139 μmol) and 2-(1-chloro-2,2-dimethylpropyl)-1H-imidazol-3-ium chloride (87.2 mg, 417 μmol, Intermediate 543) were dissolved in THF (2 mL) and water (0.2 mL) and the mixture was stirred for 2 h at 0° C. The reaction mixture was diluted with ethyl acetate and washed twice with a saturated sodium bicarbonate solution and water. The organic layer was washed with brine, dried and concentrated. The residue was purified by column chromatography (silica gel NH2, hexane/ethyl acetate (0-100%) and ethyl acetate/ethanol (0-100%) gradient). The combined fractions were purified by preparative HPLC to give 7.70 mg (95% purity, 11% yield) of the title compound.

LC-MS (Method 1): Rₜ=1.12 min; MS (ESIpos): m/z=460 [M+H]⁺

¹H-NMR (400 MHz, DMSO-d6) δ [ppm]: 0.990 (16.00), 0.994 (15.51), 1.232 (0.72), 1.817 (0.68), 1.834 (1.09), 1.852 (0.71), 2.254 (0.63), 2.273 (0.67), 2.332 (0.92), 2.435 (0.48), 2.518 (4.55), 2.523 (3.42), 2.756 (0.52), 2.779 (0.79), 2.786 (0.49), 2.803 (0.77), 2.813 (0.54), 2.825 (0.92), 2.835 (0.48), 2.858 (0.82), 2.881 (0.51), 2.957 (0.67), 2.980 (0.53), 3.029 (0.42), 3.035 (0.40), 3.520 (1.56), 3.539 (1.48), 4.045 (0.66), 4.063 (1.00), 4.080 (1.41), 4.097 (0.64), 6.012 (2.62), 6.295 (3.23), 6.511 (3.13), 7.035 (0.54), 7.953 (0.90), 7.958 (0.91), 7.976 (0.93), 7.981 (0.92), 8.526 (0.83), 8.530 (0.82), 8.548 (0.85), 8.552 (0.82).

### Example 728

### 5-{1-[cyclopropyl(1H-imidazol-2-yl)methyl]-5′,6′-dihydrospiro[azetidine-3,4′-pyrrolo[1,2-b]pyrazol]-2′-yl}-3-(trifluoromethyl)pyridin-2-amine (Enantiomer 1 and Enantiomer 2)

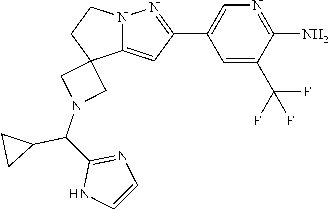 [SMILES: Nc1ncc(-c2cc3n(n2)CCC32CN(C(c3ncc[nH]3)C3CC3)C2)cc1C(F)(F)F]

5-(5′,6′-Dihydrospiro[azetidine-3,4′-pyrrolo[1,2-b]pyrazol]-2′-yl)-3-(trifluoromethyl)pyridin-2-amine (210 mg, 679 μmol, Intermediate 526) and cyclopropyl(1H-imidazol-2-yl)methanone (92.4 mg, 679 μmol) were dissolved in methanol (6.3 mL). N,N-Diisopropylethylamine (470 μL, 2.7 mmol) and titanium(IV) isopropoxide (600 μL, 2.0 mmol) were added and the mixture was stirred for 16 h at 60° C. The suspension was cooled to room temperature and sodium cyanoborohydride (128 mg, 2.04 mmol) was added and the mixture was stirred for 3 h at 60° C. The mixture was diluted with water (1 mL) and stirred for 30 min. The suspension was diluted with methanol and filtered over celite. The filtrate was concentrated under reduced pressure. The residue was purified by column chromatography (silica gel, dichloromethane/methanol (0-10%) gradient). The combined fractions were dissolved in dichloromethane/methanol and washed twice with a saturated sodium bicarbonate solution. The organic layer was dried over a hydrophobic filter and concentrated to give 120 mg (100% purity, 41% yield) of the title compound.

LC-MS (Method 1): Rₜ=0.92 min; MS (ESIpos): m/z=430 [M+H]⁺

¹H-NMR (400 MHz, DMSO-d6) δ [ppm]: 0.034 (0.59), 0.046 (0.60), 0.058 (0.46), 0.260 (0.43), 0.272 (0.57), 0.283 (0.49), 0.385 (0.49), 0.397 (0.66), 0.409 (0.60), 0.420 (0.40), 0.541 (0.42), 0.553 (0.54), 1.040 (0.53), 1.050 (0.44), 1.062 (0.52), 1.155 (3.67), 1.173 (8.21), 1.190 (4.30), 1.233 (0.77), 1.988 (16.00), 2.332 (1.00), 2.336 (0.43), 2.518 (4.84), 2.523 (3.46), 2.673 (1.01), 2.678 (0.43), 2.727 (1.68), 2.749 (1.65), 2.780 (0.81), 2.784 (0.75), 2.796 (1.45), 2.803 (1.45), 2.815 (0.77), 2.820 (0.84), 3.231 (0.83), 3.249 (1.10), 3.320 (2.17), 3.349 (1.48), 3.534 (1.02), 3.551 (0.88), 4.000 (1.12), 4.017 (3.39), 4.035 (3.33), 4.053 (1.08), 4.069 (1.31), 4.086 (2.83), 4.104 (1.28), 5.759 (3.77), 6.537 (3.37), 6.631 (7.48), 6.765 (0.92), 7.020 (0.86), 8.007 (1.89), 8.013 (1.91), 8.591 (1.79), 8.595 (1.75), 11.819 (0.87).

### Example 729

### 5-{1-[cyclopropyl(1H-imidazol-2-yl)methyl]-5′,6′-dihydrospiro[azetidine-3,4′-pyrrolo[1,2-b]pyrazol]-2′-yl}-3-(trifluoromethyl)pyridin-2-amine (Enantiomer 1)

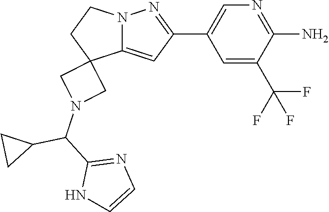 [SMILES: Nc1ncc(-c2cc3n(n2)CCC32CN(C(c3ncc[nH]3)C3CC3)C2)cc1C(F)(F)F]

The title compound from Example 728 was separated into enantiomers by preparative chiral HPLC to give 47.1 mg (96% purity) of the title compound (enantiomer 1 Rₜ=5.2-6.9 min).

Preparative Chiral HPLC Method:

Instrument: Sepiatec: Prep SFC100; Column: Chiral Art Cellulose-SB 5μ, 250×50; eluent A: CO2; eluent B: 2-propanol+0.4 vol % diethylamine; isocratic: 20% B; flow: 100 mL/min; temperature: 40° C.; BPR: 150 bar; UV: 271 nm

Analytical Chiral HPLC Method:

Instrument: Waters Acquity UPC2 QDA; Column: Chiral Art Cellulose-SB 3μ, 100×4.6; eluent A: CO2; eluent B: 2-propanol+0.4 vol % diethylamine; isocratic: 20% B; flow: 4 mL/min; temperature: 40° C.; BPR: 100 bar; UV: 280 nm

Analytical chiral HPLC: Rₜ=1.50 min (ee value: =100%)

[α]²⁰_{D}: +13.5° (c=1.00, methanol)

LC-MS (Method 1): Rₜ=0.91 min; MS (ESIpos): m/z=430 [M+H]⁺

¹H NMR (400 MHz, DMSO-d₆) δ ppm: 0.04 (dd, 1H) 0.21-0.33 (m, 1H) 0.36-0.45 (m, 1H) 0.50-0.60 (m, 1H) 1.03 (d, 2H) 2.74 (d, 1H) 2.80 (td, 2H) 3.24 (d, 1H) 3.34-3.36 (m, 1H) 3.54 (d, 1H) 4.09 (t, 2H) 6.54 (s, 2H) 6.63 (s, 1H) 6.77 (br s, 1H) 7.02 (br s, 1H) 8.01 (d, 1H) 8.59 (d, 1H) 11.82 (br s, 1H).

### Example 730

### 5-{1-[cyclopropyl(1H-imidazol-2-yl)methyl]-5′,6′-dihydrospiro[azetidine-3,4′-pyrrolo[1,2-b]pyrazol]-2′-yl}-3-(trifluoromethyl)pyridin-2-amine (Enantiomer 2)

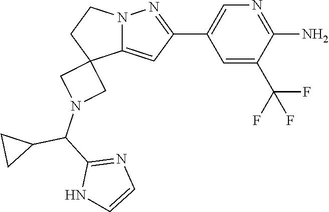 [SMILES: Nc1ncc(-c2cc3n(n2)CCC32CN(C(c3ncc[nH]3)C3CC3)C2)cc1C(F)(F)F]

For the preparation of the racemic mixture of the title compound see Example 728. The enantiomers were separated by preparative chiral HPLC (method see Example 729) to give 41.9 mg (92% purity) of the title compound (enantiomer 2 Rₜ=8.7-10.7 min).

Analytical chiral HPLC (method see Example 729): Rₜ=2.50 min (ee value: =99%)

[α]²⁰_{D}: −13.9° (c=1.00, methanol)

LC-MS (Method 1): Rₜ=0.91 min; MS (ESIpos): m/z=430 [M+H]⁺

¹H NMR (400 MHz, DMSO-d6) δ ppm: 0.04 (dq, 1H) 0.27 (tt, 1H) 0.36-0.44 (m, 1H) 0.51-0.60 (m, 1H) 1.00-1.10 (m, 1H) 1.03 (d, 1H) 2.74 (d, 1H) 2.80 (td, 2H) 3.24 (d, 1H) 3.35 (br s, 1H) 3.54 (d, 1H) 4.09 (t, 2H) 6.54 (s, 2H) 6.63 (s, 1H) 6.76 (s, 1H) 7.02 (s, 1H) 8.01 (d, 1H) 8.59 (d, 1H) 11.82 (br s, 1H).

### Example 731

### 5-{(3R)-1-[2,2-difluoro-1-(1H-imidazol-2-yl)ethyl]-5′,6′-dihydrospiro[pyrrolidine-3,4′-pyrrolo[1,2-b]pyrazol]-2′-yl}-3-(trifluoromethyl)pyridin-2-amine (Diastereomer 1 and Diastereomer 2)

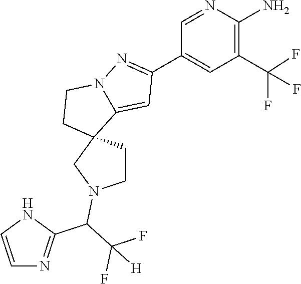 [SMILES: Nc1ncc(-c2cc3n(n2)CC[C@@]32CCN(C(c3ncc[nH]3)C(F)F)C2)cc1C(F)(F)F]

5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (410 mg, 1.14 mmol) and 2,2-difluoro-1-(1H-imidazol-2-yl)ethan-1-one (250 mg, 1.71 mmol) were dissolved in methanol (10 mL). N,N-Diisopropylethylamine (790 μL, 4.6 mmol) and titanium(IV) isopropoxide (1.0 mL, 3.4 mmol) were added and the mixture was stirred for 3 h at 60° C. Sodium cyanoborohydride (215 mg, 3.42 mmol) was added and the mixture was stirred over night at 60° C. Titanium(IV) isopropoxide (0.33 mL, 1.1 mmol) and sodium cyanoborohydride (71 mg, 1.14 mmol) were added and the mixture was stirred over night at 60° C. The suspension was filtered. The filtrate was diluted with ethyl acetate and extracted with a saturated sodium bicarbonate solution and water. The organic layer was washed with brine, dried and concentrated. The residue was purified by column chromatography (silica gel NH2, hexane/ethyl acetate (0-100%) and ethyl acetate/ethanol (0-100%) gradient). The combined fractions were purified by preparative HPLC to give 6.20 mg (95% purity, 1% yield) of the title compound.

LC-MS (Method 1): Rₜ=0.94 min; MS (ESIpos): m/z=454 [M+H]⁺

¹H-NMR (400 MHz, DMSO-d6) δ [ppm]: 0.852 (0.70), 1.233 (2.05), 1.924 (2.96), 1.941 (3.92), 1.959 (1.84), 1.986 (0.51), 2.005 (0.43), 2.336 (1.32), 2.402 (0.55), 2.416 (1.02), 2.435 (1.69), 2.452 (1.90), 2.465 (2.20), 2.518 (16.00), 2.522 (10.63), 2.660 (1.35), 2.690 (2.03), 2.701 (0.52), 2.713 (2.55), 2.729 (1.62), 2.740 (1.82), 2.752 (3.44), 2.775 (3.07), 2.799 (1.08), 2.902 (0.52), 2.930 (2.35), 2.953 (2.00), 2.974 (0.93), 2.997 (0.88), 4.072 (2.32), 4.087 (3.47), 4.107 (2.39), 4.126 (1.15), 4.143 (1.28), 4.157 (1.06), 4.169 (0.88), 4.183 (0.44), 5.759 (7.07), 6.257 (1.09), 6.271 (1.40), 6.276 (8.15), 6.313 (8.89), 6.395 (1.72), 6.408 (1.82), 6.523 (9.10), 6.547 (1.21), 6.905 (2.30), 6.908 (3.95), 6.912 (2.34), 6.923 (2.41), 6.927 (4.20), 6.930 (2.45), 7.149 (4.37), 7.153 (5.59), 7.157 (4.24), 7.983 (5.15), 8.556 (2.57), 8.562 (4.49), 12.103 (2.17).

### Example 732

### 5-{(3R)-1-[2,2-difluoro-1-(1H-imidazol-2-yl)ethyl]-5′,6′-dihydrospiro[pyrrolidine-3,4′-pyrrolo[1,2-b]pyrazol]-2′-yl}-3-(trifluoromethyl)pyridin-2-amine (Diastereomer 1)

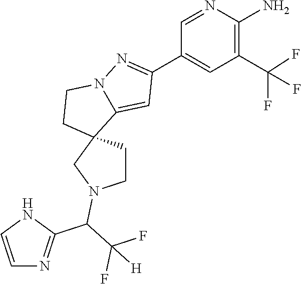 [SMILES: Nc1ncc(-c2cc3n(n2)CC[C@@]32CCN(C(c3ncc[nH]3)C(F)F)C2)cc1C(F)(F)F]

The title compound from Example 731 was separated into diastereomers by preparative chiral HPLC (diastereomer 1 Rₜ=3.6-5.0 min).

Preparative Chiral HPLC Method:

Instrument: Sepiatec: Prep SFC100; Column: Chiralpak IG 5μ, 250×50; eluent A: CO2; eluent B: methanol+0.2 vol % aqueous ammonia (32%); isocratic: 30% B; flow: 100 mL/min; temperature: 40° C.; BPR: 150 bar; UV: 271 nm

Analytical Chiral HPLC Method:

Instrument: Waters Acquity UPC2 QDA; Column: Chiralpak IG 3μ, 100×4.6; eluent A: CO2; eluent B: methanol+0.2 vol % aqueous ammonia (32%); isocratic: 30% B; flow: 4 mL/min; temperature: 40° C.; BPR: 100 bar; UV: 280 nm

Analytical chiral HPLC: Rₜ=1.04 min

The isolated fraction was purified by preparative HPLC to give 9.50 mg (95% purity) of the title compound.

LC-MS (Method 1): Rₜ=0.95 min; MS (ESIpos): m/z=454 [M+H]⁺

¹H-NMR (400 MHz, DMSO-d6) δ [ppm]: 0.852 (0.43), 1.154 (3.84), 1.172 (7.98), 1.190 (3.89), 1.233 (1.28), 1.399 (0.90), 1.418 (0.96), 1.893 (0.44), 1.909 (1.68), 1.917 (1.76), 1.926 (2.78), 1.935 (2.79), 1.942 (2.92), 1.952 (1.77), 1.968 (0.53), 1.988 (13.43), 2.318 (0.83), 2.385 (0.54), 2.404 (1.00), 2.418 (1.76), 2.437 (2.43), 2.452 (2.40), 2.468 (3.65), 2.518 (10.04), 2.523 (6.77), 2.692 (3.44), 2.715 (4.05), 2.739 (1.45), 2.758 (1.81), 2.777 (0.76), 2.900 (0.88), 2.919 (1.99), 2.930 (4.14), 2.953 (3.30), 3.999 (0.99), 4.017 (2.92), 4.035 (2.86), 4.042 (0.44), 4.053 (1.02), 4.068 (2.12), 4.074 (2.09), 4.087 (3.64), 4.089 (3.56), 4.103 (2.39), 4.108 (2.56), 4.120 (1.35), 4.137 (1.20), 4.151 (1.10), 4.165 (0.97), 4.178 (0.73), 5.332 (0.62), 6.263 (0.89), 6.276 (0.93), 6.317 (16.00), 6.401 (1.55), 6.414 (1.63), 6.527 (8.24), 6.553 (1.05), 7.058 (1.55), 7.983 (4.67), 7.988 (4.71), 8.564 (4.35), 8.568 (4.29).

### Example 733

### 5-{(3R)-1-[2,2-difluoro-1-(1H-imidazol-2-yl)ethyl]-5′,6′-dihydrospiro[pyrrolidine-3,4′-pyrrolo[1,2-b]pyrazol]-2′-yl}-3-(trifluoromethyl)pyridin-2-amine (Diastereomer 2)

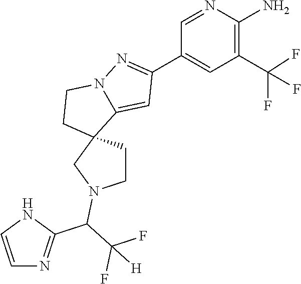 [SMILES: Nc1ncc(-c2cc3n(n2)CC[C@@]32CCN(C(c3ncc[nH]3)C(F)F)C2)cc1C(F)(F)F]

For the preparation of the diastereomeric mixture of the title compound see Example 731. The diastereomers were separated by preparative chiral HPLC (method see Example 732) (diastereomer 2 Rₜ=8.5-11.4 min).

Analytical chiral HPLC (method see Example 732): Rₜ=3.11 min

The isolated fraction was purified by preparative HPLC to give 6.80 mg (95% purity) of the title compound.

LC-MS (Method 1): Rₜ=0.95 min; MS (ESIpos): m/z=454 [M+H]⁺

¹H-NMR (400 MHz, DMSO-d6) δ [ppm]: 0.852 (0.51), 1.232 (1.68), 1.924 (2.36), 1.941 (5.03), 1.959 (2.81), 2.337 (0.73), 2.405 (0.56), 2.425 (0.90), 2.437 (1.50), 2.453 (1.87), 2.456 (2.13), 2.518 (9.70), 2.523 (6.63), 2.678 (0.75), 2.701 (0.72), 2.719 (1.95), 2.729 (2.39), 2.741 (2.12), 2.752 (4.84), 2.776 (4.73), 2.799 (1.92), 2.959 (0.76), 2.975 (1.56), 2.997 (1.47), 3.015 (0.59), 4.051 (0.50), 4.054 (0.53), 4.061 (1.73), 4.073 (1.93), 4.077 (2.52), 4.080 (2.60), 4.087 (2.41), 4.092 (2.36), 4.096 (2.00), 4.107 (1.79), 4.113 (1.14), 4.119 (0.72), 4.126 (1.03), 4.139 (0.95), 4.144 (1.15), 4.152 (0.92), 4.158 (1.18), 4.170 (0.84), 4.184 (0.70), 5.759 (6.26), 6.257 (0.95), 6.276 (16.00), 6.395 (1.43), 6.409 (1.52), 6.525 (7.78), 6.543 (0.75), 6.548 (1.03), 6.906 (4.07), 6.909 (6.88), 6.913 (3.94), 7.149 (4.11), 7.152 (4.68), 7.154 (4.68), 7.157 (3.84), 7.978 (4.41), 7.983 (4.45), 8.557 (4.10), 8.560 (3.97), 12.108 (2.04).

### Example 734

### 5-{(3S)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (Diastereomer 1 and Diastereomer 2)

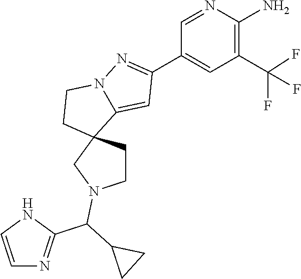 [SMILES: Nc1ncc(-c2cc3n(n2)CC[C@]32CCN(C(c3ncc[nH]3)C3CC3)C2)cc1C(F)(F)F]

5-[(3S)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (468 mg, 85% purity, 1.11 mmol) and cyclopropyl(1H-imidazol-2-yl)methanone (226 mg, 1.66 mmol) were dissolved in methanol (12 mL). N,N-Diisopropylethylamine (770 μL, 4.4 mmol) and titanium(IV) isopropoxide (980 μL, 3.3 mmol) were added and the mixture was stirred for 3 h at 60° C. The reaction mixture was allowed to reach rt, sodium cyanoborohydride (208 mg, 3.32 mmol) was added and the mixture was stirred over night at 60° C. The reaction mixture was allowed to cool down, diluted with water and stirred for 15 min. The suspension was filtered over celite and the filter cake was washed with methanol. The filtrate was concentrated. The residue was purified by column chromatography (silica gel, hexane/ethyl acetate (0-100%) and dichloromethane/methanol (0-10%) gradient). The combined fractions were purified by preparative HPLC to give 23.0 mg (99% purity, 5% yield) of the title compound.

LC-MS (Method 1): Rₜ=0.95 min; MS (ESIpos): m/z=445 [M+H]⁺

¹H NMR (400 MHz, DMSO-d₆) δ ppm: 0.04-0.10 (m, 1H) 0.29-0.40 (m, 2H) 0.57-0.68 (m, 1H) 1.18-1.29 (m, 1H) 1.92-2.03 (m, 2H) 2.42-2.47 (m, 1H) 2.52-2.71 (m, 4H) 2.74-2.80 (m, 1H) 2.90-2.97 (m, 1H) 4.04-4.13 (m, 2H) 6.44 (d, 1H) 6.52 (s, 2H) 6.75 (s, 1H) 7.03 (br d, 1H) 8.00 (s, 1H) 8.59 (d, 1H) 11.78 (br s, 1H).

### Example 735

### 5-{(3S)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (Diastereomer 1)

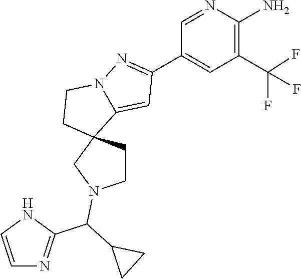 [SMILES: Nc1ncc(-c2cc3n(n2)CC[C@]32CCN(C(c3ncc[nH]3)C3CC3)C2)cc1C(F)(F)F]

The title compound from Example 734 was separated into diastereomers by preparative chiral HPLC (diastereomer 1 Rₜ=3.7-4.5 min).

Preparative Chiral HPLC Method:

Instrument: Sepiatec: Prep SFC100; Column: Chiral Art Amylose-SA 5μ, 250×50; eluent A: CO2; eluent B: 2-propanol+0.4 vol % diethylamine; isocratic: 30% B; flow: 100 mL/min; temperature: 40° C.; BPR: 150 bar; UV: 271 nm

Analytical Chiral HPLC Method:

Instrument: Waters Acquity UPC2 QDA; Column: Chiral Art Amylose-SA 3μ, 100×4.6; eluent A: CO2; eluent B: 2-propanol+0.4 vol % diethylamine; isocratic: 30% B; flow: 4 mL/min; temperature: 40° C.; BPR: 100 bar; UV: 280 nm

Analytical chiral HPLC: Rₜ=1.31 min

The crude fraction was purified by preparative HPLC to give 67.0 mg (95% purity, 32% yield) of the title compound.

[α]²⁰_{D}: −45.9° (c=1.00, DMSO)

LC-MS (Method 1): Rₜ=0.95 min; MS (ESIpos): m/z=445 [M+H]⁺

¹H-NMR (400 MHz, DMSO-d6) δ [ppm]: 0.056 (0.73), 0.069 (1.26), 0.083 (1.43), 0.095 (0.79), 0.301 (0.66), 0.309 (1.18), 0.321 (2.44), 0.330 (2.66), 0.342 (2.17), 0.354 (0.92), 0.605 (0.93), 0.615 (0.87), 0.620 (0.89), 0.627 (1.22), 0.631 (1.10), 0.634 (0.99), 0.646 (0.63), 1.027 (11.52), 1.042 (12.24), 1.206 (0.50), 1.213 (0.63), 1.217 (0.63), 1.226 (1.07), 1.236 (0.93), 1.249 (0.98), 1.256 (0.56), 1.261 (0.55), 1.268 (0.46), 1.929 (0.57), 1.942 (1.15), 1.959 (2.03), 1.976 (1.72), 1.991 (1.27), 1.996 (1.42), 2.011 (1.21), 2.022 (0.57), 2.028 (0.53), 2.043 (0.42), 2.327 (0.40), 2.444 (0.61), 2.461 (1.36), 2.476 (1.52), 2.523 (1.66), 2.568 (2.20), 2.583 (3.13), 2.601 (1.92), 2.616 (1.41), 2.634 (0.67), 2.654 (3.77), 2.665 (0.51), 2.669 (0.69), 2.677 (3.72), 2.752 (0.75), 2.773 (1.56), 2.789 (1.28), 2.795 (0.87), 2.811 (0.54), 2.931 (3.14), 2.954 (2.77), 4.085 (2.64), 4.102 (5.03), 4.120 (2.60), 6.435 (0.53), 6.445 (16.00), 6.523 (7.39), 6.752 (4.89), 7.019 (4.28), 8.000 (4.21), 8.005 (4.26), 8.584 (3.93), 8.588 (3.87), 11.794 (1.79).

### Example 736

### 5-{(3S)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (Diastereomer 2)

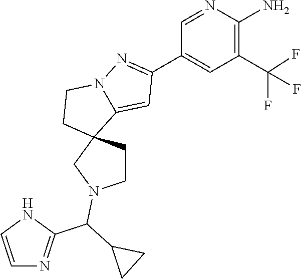 [SMILES: Nc1ncc(-c2cc3n(n2)CC[C@]32CCN(C(c3ncc[nH]3)C3CC3)C2)cc1C(F)(F)F]

For the preparation of the diastereomeric mixture of the title compound see Example 734. The diastereomers were separated by preparative chiral HPLC (method see Example 735) (diastereomer 2 Rₜ=4.7-5.6 min).

Analytical chiral HPLC (method see Example 735): Rₜ=1.93 min

The crude fraction was purified by preparative HPLC to give 52.0 mg (95% purity, 25% yield) of the title compound.

[α]²⁰_{D}: −34.0° (c=1.00, DMSO)

LC-MS (Method 1): Rₜ=0.95 min; MS (ESIpos): m/z=445 [M+H]⁺

¹H-NMR (400 MHz, DMSO-d6) δ [ppm]: 0.058 (0.84), 0.070 (1.27), 0.083 (1.39), 0.094 (1.07), 0.106 (0.47), 0.298 (0.61), 0.308 (0.99), 0.319 (1.27), 0.330 (1.43), 0.341 (0.97), 0.347 (0.97), 0.351 (1.03), 0.358 (1.22), 0.369 (1.48), 0.381 (1.34), 0.392 (0.88), 0.596 (0.48), 0.606 (0.74), 0.610 (0.66), 0.618 (1.07), 0.621 (1.00), 0.629 (1.18), 0.641 (0.97), 0.652 (0.60), 0.984 (0.56), 0.990 (0.47), 1.000 (0.54), 1.027 (7.26), 1.042 (7.81), 1.086 (0.45), 1.196 (0.57), 1.207 (0.69), 1.216 (1.17), 1.228 (1.17), 1.239 (1.14), 1.249 (0.62), 1.259 (0.52), 1.949 (0.45), 1.959 (1.52), 1.962 (1.34), 1.972 (1.51), 1.981 (2.90), 1.986 (2.18), 1.991 (2.09), 1.999 (1.63), 2.005 (1.72), 2.327 (0.44), 2.444 (0.52), 2.460 (0.73), 2.463 (0.96), 2.476 (1.73), 2.518 (2.12), 2.523 (1.59), 2.564 (2.31), 2.572 (5.32), 2.593 (1.90), 2.611 (0.69), 2.669 (0.52), 2.673 (0.43), 2.687 (3.99), 2.711 (3.84), 2.746 (3.71), 2.770 (2.98), 2.908 (0.73), 2.922 (1.13), 2.927 (1.21), 2.945 (1.28), 2.964 (0.61), 4.017 (0.46), 4.033 (0.61), 4.037 (0.73), 4.045 (1.52), 4.052 (0.80), 4.060 (1.97), 4.065 (2.82), 4.080 (2.93), 4.086 (1.89), 4.093 (0.84), 4.100 (1.52), 4.107 (0.78), 4.112 (0.61), 4.127 (0.44), 6.435 (16.00), 6.445 (0.42), 6.522 (7.92), 6.754 (4.31), 7.033 (3.64), 7.998 (4.50), 8.003 (4.58), 8.585 (4.18), 8.589 (4.16), 11.800 (1.85).

### Example 737

### 5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine (Diastereomer 1 and Diastereomer 2)

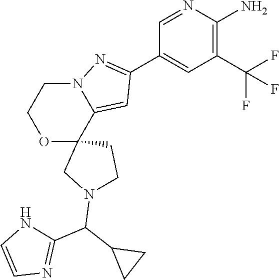 [SMILES: Nc1ncc(-c2cc3n(n2)CCO[C@@]32CCN(C(c3ncc[nH]3)C3CC3)C2)cc1C(F)(F)F]

5-[(3′R)-6,7-Dihydrospiro[pyrazolo[5,1-c][1,4]oxazine-4,3′-pyrrolidin]-2-yl]-3-(trifluoromethyl)pyridin-2-amine hydrogen chloride (1/1) (546 mg, 70% purity, 1.02 mmol) and cyclopropyl(1H-imidazol-2-yl)methanone (208 mg, 1.53 mmol) were dissolved in methanol (11 mL). N,N-Diisopropylethylamine (710 μL, 4.1 mmol) and titanium(IV) isopropoxide (900 μL, 3.1 mmol) were added and the mixture was stirred for 3 h at 60° C. To the cold reaction mixture was added sodium cyanoborohydride (192 mg, 3.05 mmol) and the mixture was stirred over night at 60° C. The reaction mixture was allowed to cool down, diluted with water and stirred for 15 min. The suspension was filtered over celite and the filter cake was washed with methanol. The filtrate was concentrated. The residue was purified by column chromatography (silica gel, hexane/ethyl acetate (0-100%) and dichloromethane/methanol (0-10%) gradient). 50 mg of the combined fractions were purified by preparative HPLC to give 31.0 mg (99% purity, 7% yield) of the title compound.

LC-MS (Method 1): Rₜ=0.93 min; MS (ESIpos): m/z=461 [M+H]⁺

¹H NMR (400 MHz, DMSO-d₆) δ ppm: 11.84-11.91 (m, 1H), 8.62 (d, 1H), 8.03 (d, 1H), 6.99-7.05 (m, 1H), 6.75 (d, 1H), 6.62-6.64 (m, 1H), 6.58 (s, 2H), 3.93-4.10 (m, 4H), 2.91-3.22 (m, 1H), 2.76-2.86 (m, 1H), 2.55-2.70 (m, 3H), 2.06-2.22 (m, 2H), 1.18-1.29 (m, 1H), 0.60-0.69 (m, 1H), 0.28-0.41 (m, 2H), 0.02-0.11 (m, 1H).

### Example 738

### 5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine (Diastereomer 1)

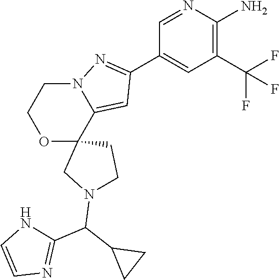 [SMILES: Nc1ncc(-c2cc3n(n2)CCO[C@@]32CCN(C(c3ncc[nH]3)C3CC3)C2)cc1C(F)(F)F]

The title compound from Example 737 was separated into diastereomers by preparative chiral HPLC (diastereomer 1 Rₜ=3.6-4.8 min).

Preparative Chiral HPLC Method:

Instrument: Sepiatec: Prep SFC100; Column: Chiral Art Amylose-SA 5μ, 250×50; eluent A: CO2; eluent B: 2-propanol+0.4 vol % diethylamine; isocratic: 30% B; flow: 100 mL/min; temperature: 40° C.; BPR: 150 bar; UV: 267 nm

Analytical Chiral HPLC Method:

Instrument: Waters Acquity UPC2 QDA; Column: Chiral Art Amylose-SA 3μ, 100×4.6; eluent A: CO2; eluent B: 2-propanol+0.4 vol % diethylamine; isocratic: 30% B; flow: 4 mL/min; temperature: 40° C.; BPR: 100 bar; UV: 254 nm

Analytical chiral HPLC: Rₜ=1.20 min

The crude fraction was purified by preparative HPLC to give 43.0 mg (95% purity, 20% yield) of the title compound.

[α]²⁰_{D}: +38.7° (c=1.00, DMSO)

LC-MS (Method 1): Rₜ=0.93 min; MS (ESIpos): m/z=461 [M+H]⁺

¹H-NMR (400 MHz, DMSO-d6) δ [ppm]: 0.041 (0.88), 0.054 (1.30), 0.066 (1.40), 0.078 (1.08), 0.090 (0.44), 0.301 (0.62), 0.312 (1.01), 0.323 (1.25), 0.333 (1.46), 0.345 (1.40), 0.355 (1.60), 0.368 (1.49), 0.380 (1.34), 0.391 (0.86), 0.616 (0.49), 0.625 (0.76), 0.637 (1.11), 0.648 (1.17), 0.660 (0.97), 0.670 (0.58), 0.984 (0.90), 0.990 (0.57), 1.000 (0.81), 1.026 (12.85), 1.041 (11.94), 1.086 (0.49), 1.137 (0.91), 1.214 (0.57), 1.227 (0.78), 1.235 (1.20), 1.246 (1.05), 1.258 (1.08), 1.278 (0.48), 1.900 (0.40), 1.959 (1.10), 2.068 (0.44), 2.074 (0.46), 2.083 (0.74), 2.087 (0.77), 2.102 (1.42), 2.116 (1.67), 2.136 (1.00), 2.148 (0.88), 2.166 (1.65), 2.181 (1.46), 2.198 (0.78), 2.215 (0.45), 2.327 (0.52), 2.518 (1.53), 2.523 (1.18), 2.558 (0.58), 2.578 (1.29), 2.596 (1.36), 2.615 (0.57), 2.670 (4.28), 2.693 (3.71), 2.774 (2.56), 2.800 (3.23), 2.922 (3.83), 2.934 (0.92), 2.948 (3.85), 2.973 (1.29), 2.990 (0.58), 3.272 (0.43), 3.387 (0.90), 3.754 (0.45), 3.769 (0.59), 3.785 (0.43), 3.941 (0.67), 3.958 (1.12), 3.973 (2.29), 3.979 (1.69), 3.992 (2.76), 4.004 (1.84), 4.025 (0.95), 4.029 (0.88), 4.037 (0.74), 4.051 (3.41), 4.064 (4.25), 4.078 (1.66), 6.578 (7.69), 6.627 (16.00), 8.030 (4.66), 8.035 (4.74), 8.616 (4.34), 8.619 (4.30).

### Example 739

### 5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine (Diastereomer 2)

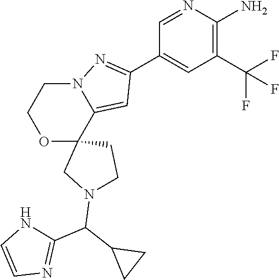 [SMILES: Nc1ncc(-c2cc3n(n2)CCO[C@@]32CCN(C(c3ncc[nH]3)C3CC3)C2)cc1C(F)(F)F]

For the preparation of the diastereomeric mixture of the title compound see Example 737. The diastereomers were separated by preparative chiral HPLC (method see Example 738) (diastereomer 2 Rₜ=5.9-7.6 min).

Analytical chiral HPLC (method see Example 738): Rₜ=2.37 min

The crude fraction was purified by preparative HPLC to give 77.0 mg (95% purity, 37% yield) of the title compound.

[α]²⁰_{D}: +46.3° (c=1.00, DMSO)

LC-MS (Method 1): Rₜ=0.93 min; MS (ESIpos): m/z=461 [M+H]⁺

¹H-NMR (400 MHz, DMSO-d6) δ [ppm]: 0.039 (0.42), 0.050 (0.88), 0.062 (1.35), 0.075 (1.51), 0.085 (1.17), 0.096 (0.48), 0.298 (0.68), 0.308 (1.10), 0.319 (1.36), 0.329 (1.89), 0.343 (1.54), 0.348 (2.29), 0.356 (1.73), 0.368 (1.46), 0.379 (0.94), 0.612 (0.52), 0.621 (0.84), 0.633 (1.24), 0.641 (1.26), 0.652 (1.02), 0.656 (1.08), 0.667 (0.63), 0.677 (0.42), 0.984 (0.68), 0.990 (0.46), 1.000 (0.76), 1.026 (2.30), 1.041 (2.10), 1.086 (0.43), 1.137 (0.52), 1.199 (0.57), 1.210 (0.72), 1.221 (1.25), 1.231 (1.17), 1.242 (1.15), 1.254 (0.62), 1.263 (0.55), 1.959 (0.92), 2.064 (0.46), 2.074 (1.49), 2.083 (0.92), 2.097 (1.42), 2.115 (1.95), 2.133 (1.12), 2.142 (0.96), 2.159 (1.76), 2.175 (1.60), 2.191 (0.79), 2.208 (0.48), 2.327 (0.52), 2.518 (2.04), 2.523 (1.42), 2.566 (0.65), 2.583 (1.56), 2.605 (1.61), 2.623 (0.71), 2.653 (7.61), 2.665 (0.80), 2.669 (1.20), 2.677 (5.75), 2.794 (0.78), 2.814 (1.46), 2.831 (1.38), 2.850 (0.61), 3.183 (3.29), 3.209 (2.96), 3.387 (0.65), 4.004 (1.39), 4.021 (3.94), 4.031 (4.50), 4.063 (4.40), 4.072 (3.68), 4.077 (3.67), 4.090 (1.53), 6.578 (8.18), 6.621 (0.58), 6.630 (16.00), 6.850 (0.59), 6.869 (0.53), 8.028 (4.92), 8.033 (5.00), 8.614 (4.63), 8.618 (4.56).

### Example 740

### 2-amino-5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}nicotinonitrile (Diastereomer 1 and Diastereomer 2)

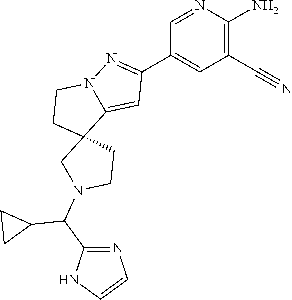 [SMILES: N#Cc1cc(-c2cc3n(n2)CC[C@@]32CCN(C(c3ncc[nH]3)C3CC3)C2)cnc1N]

2-Amino-5-[(3R)-5′,6′-dihydrospiro[pyrrolidine-3,4′-pyrrolo[1,2-b]pyrazol]-2′-yl]pyridine-3-carbonitrile hydrogen chloride (1/1) (200 mg, 631 μmol, Intermediate 528), cyclopropyl(1H-imidazol-2-yl)methanone (129 mg, 947 μmol), N,N-diisopropylethylamine (440 μL, 2.5 mmol) and titanium(IV) isopropoxide (560 μL, 1.9 mmol) were dissolved in methanol (6 mL) and stirred for 4 h at 60° C. The reaction mixture was allowed to cool down, sodium cyanoborohydride (99.2 mg, 1.58 mmol) was added and the mixture was stirred over night at 60° C. The reaction mixture was allowed to cool down, diluted with water and stirred for 10 min. The suspension was filtered over celite and the filter cake was washed with methanol. The filtrate was extracted twice with ethyl acetate. The combined organic layers were dried over a hydrophobic filter and concentrated. The residue was purified by column chromatography (silica gel, dichloromethane/methanol (0-10%) to give 167 mg (98% purity, 65% yield) of the title compound.

LC-MS (Method 1): Rₜ=0.82 min; MS (ESIpos): m/z=401 [M+H]⁺

¹H-NMR (400 MHz, DMSO-d6) δ [ppm]: 0.239 (1.32), 0.248 (1.31), 0.546 (0.72), 1.080 (0.82), 1.146 (1.08), 1.177 (5.77), 1.195 (5.68), 1.263 (16.00), 1.321 (0.43), 1.894 (0.91), 2.239 (0.80), 2.495 (1.44), 2.582 (1.40), 2.697 (0.57), 2.830 (0.71), 2.852 (0.68), 3.997 (1.20), 4.013 (2.22), 4.030 (1.19), 4.742 (0.51), 4.759 (0.69), 4.777 (0.47), 6.348 (2.66), 6.886 (4.94), 6.905 (2.43), 6.924 (1.35), 7.256 (0.86), 7.274 (1.55), 7.290 (0.77), 7.475 (0.81), 7.494 (0.80), 7.588 (0.82), 7.603 (1.46), 7.619 (0.80), 8.028 (2.23), 8.033 (2.31), 8.519 (2.27), 8.524 (2.26), 11.715 (0.44).

### Example 741

2-amino-5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}nicotinonitrile (Diastereomer 1)

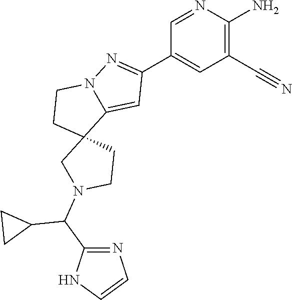 [SMILES: N#Cc1cc(-c2cc3n(n2)CC[C@@]32CCN(C(c3ncc[nH]3)C3CC3)C2)cnc1N]

The title compound from Example 740 was separated into diastereomers by preparative chiral HPLC to give 97.0 mg (90% purity) of the title compound (diastereomer 1 Rₜ=5.0-6.0 min).

Preparative Chiral HPLC Method:

Instrument: Sepiatec: Prep SFC360; Column: Chiralpak IG 5μ, 250×50; eluent A: CO2; eluent B: ethanol+0.2 vol % aqueous ammonia (32%); isocratic: 30% B; flow: 300 mL/min; temperature: 40° C.; BPR: 120 bar; UV: 276 nm

Analytical Chiral HPLC Method:

Instrument: Waters Acquity UPC2 QDA; Column: Chiralpak IG 3μ, 100×4.6; eluent A: CO2; eluent B: ethanol+0.2 vol % aqueous ammonia (32%); isocratic: 30% B; flow: 4 mL/min; temperature: 37.5° C.; BPR: 100 bar; UV: 254 nm

Analytical chiral HPLC: Rₜ=1.71 min (ee value: =98.2%)

[α]²⁰_{D}: +42.7° (c=1.00, methanol)

LC-MS (Method 1): Rₜ=0.82 min; MS (ESIpos): m/z=401 [M+H]⁺

¹H NMR (400 MHz, DMSO-d₆) δ ppm: 11.80 (br s, 1H), 8.61 (d, 1H), 8.12 (d, 1H), 6.94-7.02 (m, 4H), 6.43 (s, 1H), 4.04-4.15 (m, 2H), 2.88-2.98 (m, 1H), 2.74-2.84 (m, 1H), 2.65-2.71 (m, 1H), 2.55-2.64 (m, 2H), 1.89-2.05 (m, 2H), 1.19-1.25 (m, 1H), 0.57-0.67 (m, 1H), 0.28-0.38 (m, 2H), 0.04-0.13 (m, 1H).

### Example 742

### 2-amino-5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}nicotinonitrile (Diastereomer 2)

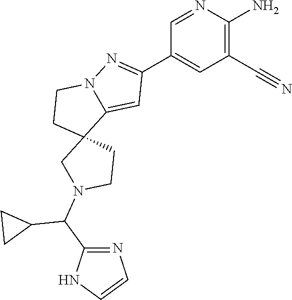 [SMILES: N#Cc1cc(-c2cc3n(n2)CC[C@@]32CCN(C(c3ncc[nH]3)C3CC3)C2)cnc1N]

For the preparation of the diastereomeric mixture of the title compound see Example 740 The diastereomers were separated by preparative chiral HPLC (method see Example 741) to give 25.0 mg (95% purity) of the title compound (diastereomer 2 Rₜ=6.8-7.77 min).

Analytical chiral HPLC (method see Example 741): Rₜ=2.33 min. (ee value: =92.2%)

[α]²⁰_{D}: +39.6° (c=1.00, methanol)

LC-MS (Method 1): Rₜ=0.82 min; MS (ESIpos): m/z=401 [M+H]⁺

¹H NMR (400 MHz, DMSO-d₆) δ ppm: 11.81 (br s, 1H), 8.61 (d, 1H), 8.11 (d, 1H), 6.97 (s, 2H), 6.43 (s, 1H), 4.01-4.14 (m, 2H), 2.87-2.99 (m, 1H), 2.65-2.80 (m, 2H), 2.53-2.63 (m, 2H), 1.93-2.03 (m, 2H), 1.17-1.28 (m, 1H), 0.59-0.68 (m, 1H), 0.27-0.42 (m, 2H), 0.04-0.13 (m, 1H).

### Example 743

### 5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(difluoromethoxy)pyridin-2-amine (Diastereomer 1 and Diastereomer 2)

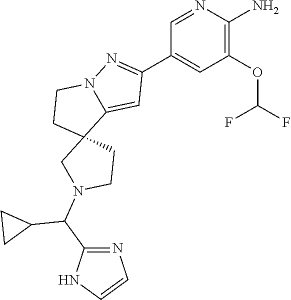 [SMILES: Nc1ncc(-c2cc3n(n2)CC[C@@]32CCN(C(c3ncc[nH]3)C3CC3)C2)cc1OC(F)F]

The compound was prepared similarly to Example 740 using 3-(difluoromethoxy)-5-[(3R)-5′,6′-dihydrospiro[pyrrolidine-3,4′-pyrrolo[1,2-b]pyrazol]-2′-yl]pyridin-2-amine hydrogen chloride (1/1) (200 mg, 559 μmol, Intermediate 529) and cyclopropyl(1H-imidazol-2-yl)methanone (114 mg, 838 μmol) to give 164 mg (100% purity, 66% yield) of the title compound.

LC-MS (Method 1): Rₜ=0.88 min; MS (ESIpos): m/z=442 [M+H]⁺

¹H-NMR (400 MHz, DMSO-d6) δ [ppm]: 0.094 (1.08), 0.107 (1.06), 0.118 (0.72), 0.310 (0.45), 0.334 (0.79), 0.349 (1.35), 0.365 (1.49), 0.377 (0.91), 0.389 (0.72), 0.402 (0.66), 0.413 (0.50), 0.621 (0.51), 0.653 (0.86), 0.667 (0.59), 1.235 (0.94), 1.254 (0.80), 1.955 (0.55), 1.974 (0.99), 1.994 (1.44), 2.002 (1.39), 2.017 (0.97), 2.322 (0.57), 2.326 (0.80), 2.332 (0.57), 2.447 (0.60), 2.465 (1.30), 2.518 (2.84), 2.522 (1.81), 2.568 (1.13), 2.589 (1.61), 2.605 (1.44), 2.620 (1.63), 2.660 (0.47), 2.664 (0.71), 2.668 (0.86), 2.673 (0.62), 2.772 (1.10), 2.793 (1.05), 2.966 (0.98), 2.989 (0.84), 3.165 (16.00), 4.041 (0.66), 4.060 (1.36), 4.081 (2.18), 4.098 (3.14), 4.116 (1.77), 6.143 (6.49), 6.349 (5.99), 6.356 (7.28), 6.971 (4.07), 6.981 (3.67), 6.992 (2.19), 6.994 (2.11), 7.176 (3.61), 7.179 (3.71), 7.361 (1.53), 7.363 (1.65), 7.606 (3.63), 7.608 (3.58), 8.201 (6.94), 8.205 (6.51).

### Example 744

### 5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(difluoromethoxy)pyridin-2-amine (Diastereomer 1)

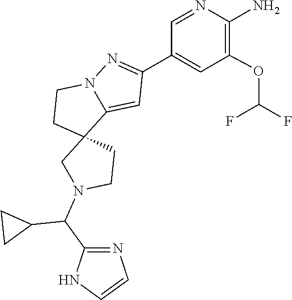 [SMILES: Nc1ncc(-c2cc3n(n2)CC[C@@]32CCN(C(c3ncc[nH]3)C3CC3)C2)cc1OC(F)F]

The title compound from Example 743 was separated into diastereomers by preparative chiral HPLC to give 93.0 mg (90% purity) of the title compound (diastereomer 1 Rₜ=4.3-5.9 min).

Preparative Chiral HPLC Method:

Instrument: Sepiatec: Prep SFC360; Column: Chiralpak IG 5μ, 250×50; eluent A: CO2; eluent B: ethanol+0.2 vol % aqueous ammonia (32%); isocratic: 30% B; flow: 100 mL/min; temperature: 40° C.; BPR: 120 bar; UV: 254 nm

Analytical Chiral HPLC Method:

Instrument: Waters Acquity UPC2 QDA; Column: Chiralpak IG 3μ, 100×4.6; eluent A: CO2; eluent B: ethanol+0.2 vol % aqueous ammonia (32%); isocratic: 30% B; flow: 4 mL/min; temperature: 37.5° C.; BPR: 100 bar; UV: 254 nm

Analytical chiral HPLC: Rₜ=1.43 min

[α]²⁰_{D}: +37.8° (c=1.00, methanol)

LC-MS (Method 1): Rₜ=0.94 min; MS (ESIpos): m/z=442 [M+H]⁺

¹H NMR (400 MHz, DMSO-d₆) δ ppm: 11.79 (br s, 1H), 8.20 (d, 1H), 7.60-7.62 (m, 1H), 7.18 (t, 1H), 6.96-7.02 (m, 2H), 6.35 (s, 1H), 6.14 (s, 2H), 4.09 (t, 2H), 2.95 (d, 1H), 2.73-2.80 (m, 1H), 2.53-2.69 (m, 4H), 1.89-2.04 (m, 2H), 1.20-1.26 (m, 1H), 0.59-0.66 (m, 1H), 0.27-0.37 (m, 2H), 0.03-0.12 (m, 1H).

### Example 745

### 5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(difluoromethoxy)pyridin-2-amine (Diastereomer 2)

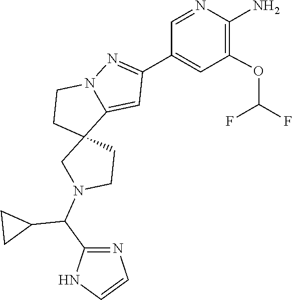 [SMILES: Nc1ncc(-c2cc3n(n2)CC[C@@]32CCN(C(c3ncc[nH]3)C3CC3)C2)cc1OC(F)F]

For the preparation of the diastereomeric mixture of the title compound see Example 743. The diastereomers were separated by preparative chiral HPLC (method see Example 744) to give 53.0 mg (95% purity) of the title compound (diastereomer 2 Rₜ=9.8-12.9 min).

Analytical chiral HPLC (method see Example 744): Rₜ=3.45 min.

[α]²⁰_{D}: +41.0° (c=1.00, methanol)

LC-MS (Method 1): Rₜ=0.94 min; MS (ESIpos): m/z=442 [M+H]⁺

¹H NMR (400 MHz, DMSO-d₆) δ ppm: 11.81 (br s, 1H), 8.20 (d, 1H), 7.61 (s, 1H), 7.18 (t, 1H), 6.69-7.08 (m, 2H), 6.34 (s, 1H), 6.14 (s, 2H), 3.99-4.13 (m, 2H), 2.90-2.98 (m, 1H), 2.68-2.78 (m, 2H), 2.54-2.63 (m, 3H), 1.91-2.05 (m, 2H), 1.18-1.26 (m, 1H), 0.58-0.68 (m, 1H), 0.27-0.43 (m, 2H), 0.03-0.13 (m, 1H).

### Example 746

### 2-amino-5-{(3R)-1-[1-(1H-imidazol-2-yl)propyl]-5′,6′-dihydrospiro[pyrrolidine-3,4′-pyrrolo[1,2-b]pyrazol]-2′-yl}nicotinonitrile (Diastereomer 1 and Diastereomer 2)

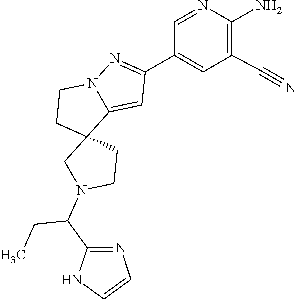 [SMILES: CCC(c1ncc[nH]1)N1CC[C@@]2(CCn3nc(-c4cnc(N)c(C#N)c4)cc32)C1]

The compound was prepared similarly to Example 740 using 2-amino-5-[(3R)-5′,6′-dihydrospiro[pyrrolidine-3,4′-pyrrolo[1,2-b]pyrazol]-2′-yl]pyridine-3-carbonitrile hydrogen chloride (1/1) (200 mg, 631 μmol, Intermediate 528) and 1-(1H-imidazol-2-yl)propan-1-one (118 mg, 947 μmol) to give 192 mg (100% purity, 78% yield) of the title compound.

LC-MS (Method 1): Rₜ=0.79 min; MS (ESIpos): m/z=389 [M+H]⁺

¹H-NMR (400 MHz, DMSO-d6) δ [ppm]: 0.715 (1.07), 0.720 (1.13), 0.734 (2.52), 0.739 (2.45), 0.752 (1.20), 0.757 (1.13), 0.826 (0.64), 1.832 (0.41), 1.876 (0.41), 1.889 (0.42), 1.895 (0.40), 1.907 (0.54), 1.917 (0.88), 1.934 (1.31), 1.951 (0.63), 2.447 (0.47), 2.456 (0.44), 2.473 (0.62), 2.518 (1.20), 2.522 (0.79), 2.626 (0.53), 2.647 (0.64), 2.664 (0.43), 2.668 (0.44), 2.679 (0.40), 2.817 (0.47), 3.165 (16.00), 3.604 (0.42), 4.070 (1.05), 4.078 (0.68), 4.088 (1.39), 4.097 (0.53), 4.105 (0.68), 6.327 (2.48), 6.352 (2.69), 6.973 (3.02), 7.032 (2.86), 7.039 (3.14), 8.103 (1.43), 8.109 (2.66), 8.115 (1.51), 8.589 (1.54), 8.594 (2.19), 8.599 (1.54).

### Example 747

### 2-amino-5-{(3R)-1-[1-(1H-imidazol-2-yl)propyl]-5′,6′-dihydrospiro[pyrrolidine-3,4′-pyrrolo[1,2-b]pyrazol]-2′-yl}nicotinonitrile (Diastereomer 1)

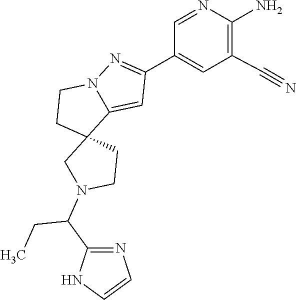 [SMILES: CCC(c1ncc[nH]1)N1CC[C@@]2(CCn3nc(-c4cnc(N)c(C#N)c4)cc32)C1]

The title compound from Example 746 was separated into diastereomers by preparative chiral HPLC to give 62.9 mg (97% purity) of the title compound (diastereomer 1 Rₜ=9.5-10.7 min).

Preparative Chiral HPLC Method:

Instrument: Sepiatec: Prep SFC100; Column: Chiral Art Cellulose-SB 5μ, 250×50; eluent A: CO2; eluent B: 2-propanol+0.4 vol % diethylamine; isocratic: 20% B; flow: 100 mL/min; temperature: 40° C.; BPR: 150 bar; UV: 276 nm

Analytical Chiral HPLC Method:

Instrument: Waters Acquity UPC2 QDA; Column: Chiral Art Cellulose-SB 3μ, 100×4.6; eluent A: CO2; eluent B: 2-propanol+0.4 vol % diethylamine; isocratic: 20% B; flow: 4 mL/min; temperature: 40° C.; BPR: 100 bar; UV: 280 nm

Analytical chiral HPLC: Rₜ=2.95 min

[α]²⁰_{D}: +52.1° (c=1.00, methanol)

LC-MS (Method 1): Rₜ=0.81 min; MS (ESIpos): m/z=389 [M+H]⁺

¹H NMR (400 MHz, DMSO-d₆) δ ppm: 11.77 (br s, 1H), 8.59 (d, 1H), 8.11 (d, 1H), 6.71-7.10 (m, 4H), 6.31 (s, 1H), 4.01-4.13 (m, 2H), 3.52 (dd, 1H), 2.79-2.90 (m, 1H), 2.63-2.69 (m, 1H), 2.55-2.62 (m, 2H), 2.38-2.46 (m, 1H), 1.73-1.95 (m, 4H), 0.74 (t, 3H).

### Example 748

### 2-amino-5-{(3R)-1-[1-(1H-imidazol-2-yl)propyl]-5′,6′-dihydrospiro[pyrrolidine-3,4′-pyrrolo[1,2-b]pyrazol]-2′-yl}nicotinonitrile (Diastereomer 2)

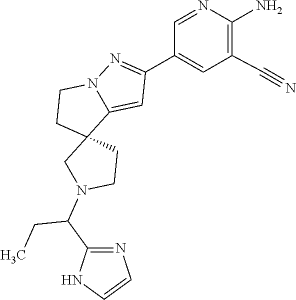 [SMILES: CCC(c1ncc[nH]1)N1CC[C@@]2(CCn3nc(-c4cnc(N)c(C#N)c4)cc32)C1]

For the preparation of the diastereomeric mixture of the title compound see Example 746. The diastereomers were separated by preparative chiral HPLC (method see Example 747) to give 62.9 mg (90% purity) of the title compound (diastereomer 2 Rₜ=11.6-13.1 min).

Analytical chiral HPLC (method see Example 747): Rₜ=3.58 min.

[α]²⁰_{D}: +69.9° (c=1.00, methanol)

LC-MS (Method 1): Rₜ=0.81 min; MS (ESIpos): m/z=389 [M+H]⁺

¹H NMR (400 MHz, DMSO-d₆) δ ppm: 11.77 (br s, 1H), 8.60 (d, 1H), 8.11 (d, 1H), 6.74-7.10 (m, 4H), 6.34 (s, 1H), 4.08 (br t, 2H), 3.52 (br dd, 1H), 2.74-2.83 (m, 2H), 2.54-2.64 (m, 2H), 2.37-2.46 (m, 1H), 1.73-1.98 (m, 4H), 0.73 (br t, 3H).

### Example 749

### 5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethoxy)pyridin-2-amine (Diastereomer 1 and Diastereomer 2)

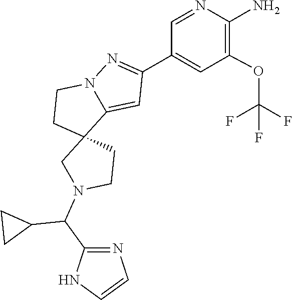 [SMILES: Nc1ncc(-c2cc3n(n2)CC[C@@]32CCN(C(c3ncc[nH]3)C3CC3)C2)cc1OC(F)(F)F]

The compound was prepared similarly to Example 740 using 5-[(3R)-5′,6′-dihydrospiro[pyrrolidine-3,4′-pyrrolo[1,2-b]pyrazol]-2′-yl]-3-(trifluoromethoxy)pyridin-2-amine hydrogen chloride (1/1) (200 mg, 532 μmol, Intermediate 527) and 1 cyclopropyl(1H-imidazol-2-yl)methanone (109 mg, 798 μmol) to give 161 mg (99% purity, 65% yield) of the title compound.

LC-MS (Method 1): Rₜ=0.95 min; MS (ESIneg): m/z=458 [M−H]⁻

¹H-NMR (400 MHz, DMSO-d6) δ [ppm]: −0.131 (0.61), 0.093 (1.22), 0.105 (1.25), 0.119 (1.36), 0.312 (0.73), 0.344 (1.02), 0.361 (1.69), 0.377 (1.85), 0.391 (0.99), 0.404 (0.90), 0.417 (0.82), 0.429 (0.69), 0.440 (0.44), 0.533 (0.62), 0.631 (0.64), 0.651 (1.07), 0.665 (1.13), 0.677 (0.76), 1.027 (0.78), 1.042 (0.78), 1.217 (1.25), 1.237 (4.49), 1.254 (4.44), 1.950 (0.41), 1.962 (0.72), 1.981 (1.35), 2.000 (1.99), 2.009 (1.72), 2.041 (0.44), 2.323 (0.69), 2.327 (0.98), 2.332 (0.70), 2.450 (0.68), 2.468 (1.58), 2.518 (4.10), 2.523 (2.46), 2.572 (1.12), 2.591 (1.64), 2.609 (1.75), 2.624 (1.80), 2.642 (1.62), 2.660 (1.12), 2.665 (1.34), 2.669 (1.48), 2.673 (1.08), 2.785 (1.36), 2.804 (1.43), 2.978 (1.19), 2.999 (1.03), 3.141 (0.95), 3.166 (16.00), 4.048 (0.86), 4.067 (1.68), 4.087 (2.69), 4.105 (3.48), 4.123 (1.82), 6.394 (6.48), 6.402 (7.86), 6.487 (8.51), 7.008 (5.93), 7.020 (5.12), 7.039 (0.41), 7.729 (2.17), 7.733 (3.80), 7.737 (3.83), 8.328 (7.14), 8.333 (6.94).

### Example 750

5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethoxy)pyridin-2-amine (Diastereomer 1)

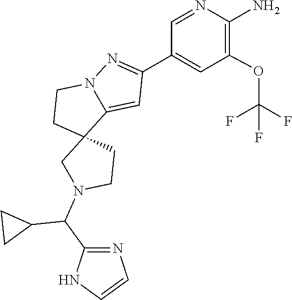 [SMILES: Nc1ncc(-c2cc3n(n2)CC[C@@]32CCN(C(c3ncc[nH]3)C3CC3)C2)cc1OC(F)(F)F]

The title compound from Example 749 was separated into diastereomers by preparative chiral HPLC to give 56.5 mg (93% purity) of the title compound (diastereomer 1 Rₜ=3.45-5.29 min).

Preparative Chiral HPLC Method:

Instrument: Sepiatec: Prep SFC100; Column: Chiralpak IG 5μ, 250×50; eluent A: CO2; eluent B: methanol+0.2 vol % aqueous ammonia (32%); isocratic: 30% B; flow: 100 mL/min; temperature: 40° C.; BPR: 150 bar; UV: 280 nm

Analytical Chiral HPLC Method:

Instrument: Waters Acquity UPC2 QDA; Column: Chiralpak IG 3μ, 100×4.6; eluent A: CO2; eluent B: methanol+0.2 vol % aqueous ammonia (32%); isocratic: 30% B; flow: 4 mL/min; temperature: 37.5° C.; BPR: 100 bar; UV: 280 nm

Analytical chiral HPLC: Rₜ=0.93 min (ee value: =98%)

[α]²⁰_{D}: +75.7° (c=1.00, methanol)

LC-MS (Method 1): Rₜ=0.97 min; MS (ESIneg): m/z=458 [M−H]⁻

¹H NMR (400 MHz, DMSO-d₆) δ ppm: 11.79 (br s, 1H), 8.33 (d, 1H), 7.74 (t, 1H), 6.94-7.08 (m, 1H), 6.75 (br s, 1H), 6.48 (s, 2H), 6.40 (s, 1H), 2.74-2.81 (m, 1H), 2.52-2.69 (m, 4H), 2.42-2.48 (m, 1H), 1.89-2.06 (m, 2H), 1.17-1.29 (m, 1H), 0.57-0.66 (m, 1H), 0.27-0.38 (m, 2H), 0.03-0.12 (m, 1H).

### Example 751

### 5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethoxy)pyridin-2-amine (Diastereomer 2)

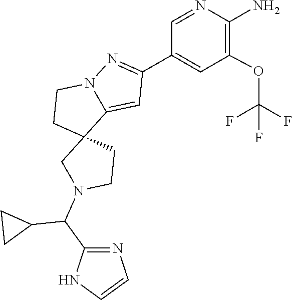 [SMILES: Nc1ncc(-c2cc3n(n2)CC[C@@]32CCN(C(c3ncc[nH]3)C3CC3)C2)cc1OC(F)(F)F]

For the preparation of the diastereomeric mixture of the title compound see Example 749 The diastereomers were separated by preparative chiral HPLC (method see Example 750) to give 47.4 mg (94% purity) of the title compound (diastereomer 2 Rₜ=5.80-7.50 min).

Analytical chiral HPLC (method see Example 750): Rₜ=1.99 min.

[α]²⁰_{D}: +40.5° (c=1.00, methanol)

LC-MS (Method 1): Rₜ=0.97 min; MS (ESIneg): m/z=458 [M−H]⁻

¹H NMR (400 MHz, DMSO-d₆) δ ppm: 11.79 (br s, 1H), 8.33 (d, 1H), 7.72-7.75 (m, 1H), 7.03 (br s, 1H), 6.75 (br s, 1H), 6.48 (s, 2H), 6.39 (s, 1H), 4.00-4.13 (m, 2H), 2.89-2.97 (m, 1H), 2.75 (d, 1H), 2.70 (d, 1H), 2.53-2.62 (m, 3H), 2.43-2.48 (m, 1H), 1.92-2.05 (m, 2H), 1.17-1.28 (m, 1H), 0.58-0.67 (m, 1H), 0.27-0.43 (m, 2H), 0.07 (dq, 1H).

### Example 752

### 3-(difluoromethoxy)-5-{(3R)-1-[1-(1H-imidazol-2-yl)propyl]-5′,6′-dihydrospiro[pyrrolidine-3,4′-pyrrolo[1,2-b]pyrazol]-2′-yl}pyridin-2-amine (Diastereomer 1 and Diastereomer 2)

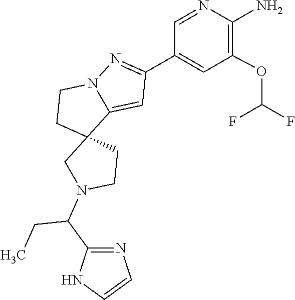 [SMILES: CCC(c1ncc[nH]1)N1CC[C@@]2(CCn3nc(-c4cnc(N)c(OC(F)F)c4)cc32)C1]

The compound was prepared similarly to Example 740 using 3-(difluoromethoxy)-5-[(3R)-5′,6′-dihydrospiro[pyrrolidine-3,4′-pyrrolo[1,2-b]pyrazol]-2′-yl]pyridin-2-amine hydrogen chloride (1/1) (413 mg, 1.15 mmol, Intermediate 529) and 1-(1H-imidazol-2-yl)propan-1-one (215 mg, 1.73 mmol) to give 405 mg (97% purity, 79% yield) of the title compound.

LC-MS (Method 1): Rₜ=0.85 min; MS (ESIpos): m/z=430 [M+H]⁺

¹H-NMR (400 MHz, DMSO-d₆) 5 [ppm]: 8.18 (t, 1H), 7.58-7.61 (s, 1H), 7.18 (t, 1H), 7.06 (d, 2H), 6.22 and 6.26 (2s, 1H), 6.14 (s, 2H), 4.02-4.11 (m, 2H), 3.58-3.67 (m, 1H), 2.59-2.94 (m, 4H), 2.39-2.47 (m, 1H), 1.75-2.00 (m, 4H), 0.70-0.77 (m, 3H).

### Example 753

### 3-(difluoromethoxy)-5-{(3R)-1-[1-(1H-imidazol-2-yl)propyl]-5′,6′-dihydrospiro[pyrrolidine-3,4′-pyrrolo[1,2-b]pyrazol]-2′-yl}pyridin-2-amine (Diastereomer 1)

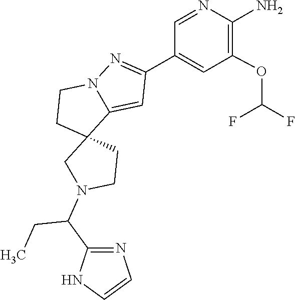 [SMILES: CCC(c1ncc[nH]1)N1CC[C@@]2(CCn3nc(-c4cnc(N)c(OC(F)F)c4)cc32)C1]

The title compound from Example 752 was separated into diastereomers by preparative chiral HPLC to give 134 mg (96% purity) of the title compound (diastereomer 1 Rₜ=9.70-11.90 min).

Preparative Chiral HPLC Method:

Instrument: Sepiatec: Prep SFC100; Column: Chiral Art Amylose-SA 5μ, 250×50; eluent A: CO2; eluent B: methanol+0.2 vol % aqueous ammonia (32%); isocratic: 10% B; flow: 100 mL/min; temperature: 40° C.; BPR: 150 bar; UV: 280 nm

Analytical Chiral HPLC Method:

Instrument: Waters Acquity UPC2 QDA; Column: Chiral Art Amylose-SA 3μ, 100×4.6; eluent A: CO2; eluent B: methanol+0.2 vol % aqueous ammonia (32%); isocratic: 20% B; flow: 4 mL/min; temperature: 37.5° C.; BPR: 100 bar; UV: 280 nm

Analytical chiral HPLC: Rₜ=1.01 min

[α]²⁰_{D}: +58.5° (c=1.00, methanol)

LC-MS (Method 1): Rₜ=0.87 min; MS (ESIpos): m/z=430 [M+H]⁺

¹H NMR (DMSO-d₆, 400 MHz): δ (ppm) 11.78 (br s, 1H), 8.19 (d, 1H), 7.59-7.61 (m, 1H), 7.18 (t, 1H), 7.04 (br s, 1H), 6.82 (br s, 1H), 6.24 (s, 1H), 6.13 (s, 2H), 4.04-4.11 (m, 2H), 3.52 (dd, 1H), 2.73-2.83 (m, 2H), 2.53-2.64 (m, 4H), 2.36-2.47 (m, 1H), 1.73-1.99 (m, 4H), 0.73 (t, 3H).

### Example 754

### 3-(difluoromethoxy)-5-{(3R)-1-[1-(1H-imidazol-2-yl)propyl]-5′,6′-dihydrospiro[pyrrolidine-3,4′-pyrrolo[1,2-b]pyrazol]-2′-yl}pyridin-2-amine (Diastereomer 2)

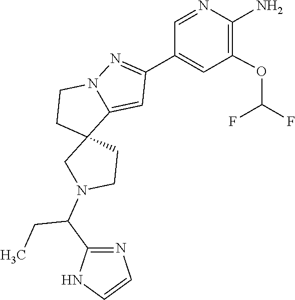 [SMILES: CCC(c1ncc[nH]1)N1CC[C@@]2(CCn3nc(-c4cnc(N)c(OC(F)F)c4)cc32)C1]

For the preparation of the diastereomeric mixture of the title compound see Example 752. The diastereomers were separated by preparative chiral HPLC (method see Example 753) to give 152 mg (99% purity) of the title compound (diastereomer 2 Rₜ=12.80-15.90 min).

Analytical chiral HPLC (method see Example 753): Rₜ=1.44 min.

[α]²⁰_{D}: +40.0° (c=1.00, methanol)

LC-MS (Method 1): Rₜ=0.86 min; MS (ESIpos): m/z=430 [M+H]⁺

¹H NMR (400 MHz, DMSO-d₆) δ ppm: 12.35 (br s, 1H), 8.18 (d, 1H), 7.58-7.61 (m, 1H), 7.18 (t, 1H), 7.06 (s, 2H), 6.22 (s, 1H), 6.14 (s, 2H), 4.01-4.12 (m, 2H), 3.59-3.67 (m, 1H), 2.87-2.95 (m, 1H), 2.60-2.75 (m, 3H), 2.40-2.46 (m, 1H), 1.76-1.98 (m, 4H), 0.74 (t, 3H).

### Example 755

### 5-{(3R)-1-[1-(1H-imidazol-2-yl)propyl]-5′,6′-dihydrospiro[pyrrolidine-3,4′-pyrrolo[1,2-b]pyrazol]-2′-yl}-3-(trifluoromethoxy)pyridin-2-amine (Diastereomer 1 and Diastereomer 2)

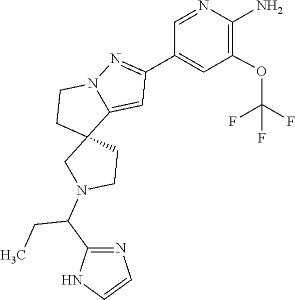 [SMILES: CCC(c1ncc[nH]1)N1CC[C@@]2(CCn3nc(-c4cnc(N)c(OC(F)(F)F)c4)cc32)C1]

The compound was prepared similarly to Example 740 using 5-[(3R)-5′,6′-dihydrospiro[pyrrolidine-3,4′-pyrrolo[1,2-b]pyrazol]-2′-yl]-3-(trifluoromethoxy)pyridin-2-amine hydrogen chloride (1/1) (200 mg, 532 μmol, Intermediate 527) and 1-(1H-imidazol-2-yl)propan-1-one (99.1 mg, 798 μmol) to give 171 mg (100% purity, 72% yield) of the title compound.

LC-MS (Method 1): Rₜ=0.93 min; MS (ESIpos): m/z=448 [M+H]⁺

¹H-NMR (400 MHz, DMSO-d6) δ [ppm]: −0.131 (0.51), 0.090 (0.51), 0.311 (0.52), 0.532 (0.51), 0.714 (4.06), 0.721 (4.36), 0.733 (9.65), 0.739 (9.23), 0.751 (4.58), 0.758 (4.07), 0.807 (2.09), 0.825 (4.92), 0.843 (2.21), 1.027 (2.44), 1.042 (2.40), 1.234 (1.88), 1.253 (1.54), 1.670 (0.41), 1.686 (0.58), 1.704 (0.77), 1.722 (0.52), 1.739 (0.49), 1.753 (0.59), 1.757 (0.54), 1.772 (1.03), 1.791 (1.09), 1.806 (1.42), 1.827 (1.65), 1.848 (1.73), 1.869 (1.80), 1.883 (1.82), 1.888 (1.68), 1.900 (1.70), 1.907 (1.61), 1.917 (3.55), 1.933 (4.89), 1.952 (2.63), 2.323 (0.89), 2.327 (1.28), 2.332 (0.86), 2.392 (0.43), 2.400 (0.44), 2.409 (1.04), 2.424 (1.28), 2.432 (1.30), 2.442 (1.86), 2.451 (1.62), 2.458 (1.35), 2.470 (1.96), 2.518 (4.81), 2.523 (3.34), 2.578 (1.11), 2.602 (1.80), 2.619 (2.12), 2.641 (2.66), 2.660 (1.11), 2.665 (1.45), 2.669 (1.63), 2.674 (1.24), 2.685 (1.42), 2.709 (0.75), 2.766 (0.44), 2.787 (0.87), 2.803 (0.80), 2.825 (1.56), 2.848 (1.14), 2.878 (0.85), 2.900 (0.75), 3.166 (16.00), 3.337 (1.07), 3.577 (1.55), 3.596 (1.48), 4.047 (1.43), 4.065 (3.90), 4.072 (2.32), 4.082 (5.30), 4.091 (1.81), 4.099 (2.40), 4.537 (0.49), 5.759 (0.85), 6.255 (10.66), 6.295 (10.65), 6.479 (11.68), 6.994 (5.16), 7.006 (10.71), 7.012 (9.94), 7.718 (2.58), 7.723 (4.34), 7.727 (4.28), 8.307 (5.79), 8.312 (8.80), 8.317 (5.84).

### Example 756

### 5-{(3R)-1-[1-(1H-imidazol-2-yl)propyl]-5′,6′-dihydrospiro[pyrrolidine-3,4′-pyrrolo[1,2-b]pyrazol]-2′-yl}-3-(trifluoromethoxy)pyridin-2-amine (Diastereomer 1)

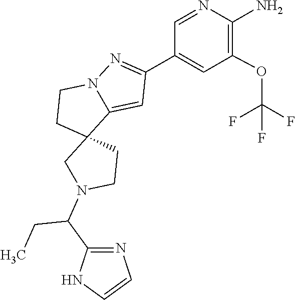 [SMILES: CCC(c1ncc[nH]1)N1CC[C@@]2(CCn3nc(-c4cnc(N)c(OC(F)(F)F)c4)cc32)C1]

The title compound from Example 755 was separated into diastereomers by preparative chiral HPLC to give 56.4 mg (100% purity) of the title compound (diastereomer 1 Rₜ=3.30-4.30 min).

Preparative Chiral HPLC Method:

Instrument: Sepiatec: Prep SFC100; Column: Chiralpak IG 5μ, 250×50; eluent A: CO2; eluent B: methanol+0.2 vol % aqueous ammonia (32%); isocratic: 30% B; flow: 100 mL/min; temperature: 40° C.; BPR: 150 bar; UV: 280 nm

Analytical Chiral HPLC Method:

Instrument: Waters Acquity UPC2 QDA; Column: Chiralpak IG 3μ, 100×4.6; eluent A: CO2; eluent B: methanol+0.2 vol % aqueous ammonia (32%); isocratic: 30% B; flow: 4 mL/min; temperature: 37.5° C.; BPR: 100 bar; UV: 280 nm

Analytical chiral HPLC: Rₜ=0.84 min

[α]²⁰_{D}: +61.4° (c=1.00, methanol)

LC-MS (Method 1): Rₜ=0.93 min; MS (ESIpos): m/z=448 [M+H]⁺

¹H NMR (400 MHz, DMSO-d₆) δ ppm: 11.77 (br s, 1H), 8.32 (d, 1H), 7.72-7.74 (m, 1H), 7.03 (br s, 1H), 6.81 (br s, 1H), 6.47 (s, 2H), 6.29 (s, 1H), 4.08 (t, 2H), 3.52 (dd, 1H), 2.73-2.82 (m, 2H), 2.53-2.64 (m, 2H), 2.37-2.47 (m, 1H), 1.73-1.98 (m, 4H), 0.73 (t, 3H).

### Example 757

### 5-{(3R)-1-[1-(1H-imidazol-2-yl)propyl]-5′,6′-dihydrospiro[pyrrolidine-3,4′-pyrrolo[1,2-b]pyrazol]-2′-yl}-3-(trifluoromethoxy)pyridin-2-amine (Diastereomer 2)

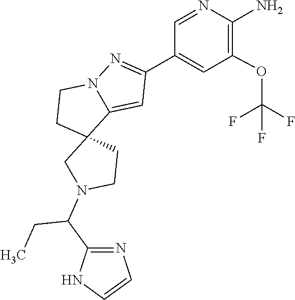 [SMILES: CCC(c1ncc[nH]1)N1CC[C@@]2(CCn3nc(-c4cnc(N)c(OC(F)(F)F)c4)cc32)C1]

For the preparation of the diastereomeric mixture of the title compound see Example 755 The diastereomers were separated by preparative chiral HPLC (method see Example 756) to give 54.7 mg (100% purity) of the title compound (diastereomer 2 Rₜ=5.02-7.00 min).

Analytical chiral HPLC (method see Example 756): Rₜ=1.37 min.

[α]²⁰_{D}: +39.2° (c=1.00, methanol)

LC-MS (Method 1): Rₜ=0.93 min; MS (ESIpos): m/z=448 [M+H]⁺

¹H NMR (400 MHz, DMSO-d₆) δ ppm: 11.78 (br s, 1H), 8.31 (d, 1H), 7.70-7.74 (m, 1H), 7.04 (br s, 1H), 6.81 (br s, 1H), 6.47 (s, 2H), 6.24 (s, 1H), 4.00-4.12 (m, 2H), 3.53 (dd, 1H), 2.81-2.89 (m, 1H), 2.64-2.69 (m, 1H), 2.55-2.63 (m, 2H), 2.38-2.47 (m, 1H), 1.72-1.96 (m, 4H), 0.74 (t, 3H).

### Example 758

### 5-{(3R)-1-[1-(1H-imidazol-2-yl)-2-methylpropyl]-5′,6′-dihydrospiro[pyrrolidine-3,4′-pyrrolo[1,2-b]pyrazol]-2′-yl}-3-(trifluoromethyl)pyridin-2-amine (Diastereomer 1 and Diastereomer 2)

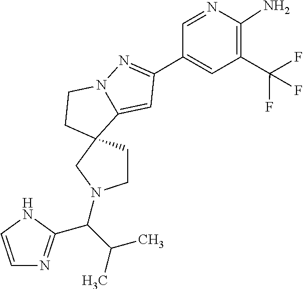 [SMILES: CC(C)C(c1ncc[nH]1)N1CC[C@@]2(CCn3nc(-c4cnc(N)c(C(F)(F)F)c4)cc32)C1]

5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (618 mg, 57% purity, 979 μmol) and 1-(1H-imidazol-2-yl)-2-methylpropan-1-one (203 mg, 1.47 mmol) were dissolved in methanol (10 mL). N,N-Diisopropylethylamine (680 μL, 3.9 mmol) and titanium(IV) isopropoxide (870 μL, 2.9 mmol) were added and the mixture was stirred for 3 h at 60° C. The reaction mixture was allowed to cool down, sodium cyanoborohydride (185 mg, 2.94 mmol) was added and stirred over night at 60° C. The reaction mixture was allowed to cool down, treated with water (2 mL) and stirred for 15 min. The suspension was filtered over celite and the filter cake was washed with methanol. The filtrate was concentrated. The crude product was purified by column chromatography (silica gel, hexane/ethyl acetate (0-100%) and dichloromethane/methanol (0-10%) gradient). 24 mg of the combined fractions were purified by preparative HPLC to give 8.00 mg (95% purity, 2% yield) of the title compound.

LC-MS (Method 1): Rₜ=1.03 min; MS (ESIpos): m/z=447 [M+H]⁺

¹H-NMR (400 MHz, DMSO-d6) δ [ppm]: 0.673 (11.60), 0.684 (12.64), 0.689 (13.35), 0.700 (11.15), 0.962 (10.62), 0.976 (15.21), 0.977 (15.31), 0.991 (11.44), 1.839 (0.57), 1.851 (1.45), 1.868 (3.68), 1.886 (5.84), 1.903 (3.13), 1.914 (1.46), 1.925 (0.61), 1.945 (0.42), 2.074 (1.76), 2.127 (0.56), 2.144 (1.50), 2.160 (2.12), 2.166 (1.81), 2.177 (1.74), 2.182 (2.17), 2.199 (1.48), 2.216 (0.53), 2.323 (1.00), 2.327 (1.39), 2.332 (0.99), 2.363 (0.50), 2.380 (1.23), 2.394 (1.41), 2.412 (2.55), 2.422 (1.62), 2.430 (1.43), 2.441 (2.13), 2.449 (1.49), 2.458 (1.71), 2.466 (3.53), 2.518 (6.70), 2.523 (4.31), 2.561 (0.86), 2.580 (4.90), 2.602 (6.58), 2.621 (2.34), 2.632 (2.09), 2.642 (1.00), 2.655 (5.09), 2.669 (6.50), 2.692 (1.70), 2.779 (0.87), 2.793 (4.47), 2.799 (3.01), 2.815 (5.90), 2.837 (1.92), 2.854 (0.67), 3.349 (8.28), 3.368 (5.06), 3.372 (5.12), 4.064 (4.76), 4.081 (9.92), 4.099 (4.84), 6.221 (16.00), 6.271 (15.66), 6.515 (13.98), 6.866 (0.88), 7.026 (0.88), 7.981 (8.47), 8.558 (8.13), 11.719 (1.97).

### Example 759

### 5-{(3R)-1-[1-(1H-imidazol-2-yl)-2-methylpropyl]-5′,6′-dihydrospiro[pyrrolidine-3,4′-pyrrolo[1,2-b]pyrazol]-2′-yl}-3-(trifluoromethyl)pyridin-2-amine (Diastereomer 1)

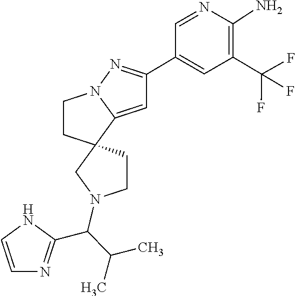 [SMILES: CC(C)C(c1ncc[nH]1)N1CC[C@@]2(CCn3nc(-c4cnc(N)c(C(F)(F)F)c4)cc32)C1]

The title compound from Example 758 was separated into diastereomers by preparative chiral HPLC (diastereomer 1 Rₜ=5.71-7.08 min).

Preparative Chiral HPLC Method:

Instrument: PrepCon Labomatic HPLC-2; Column: Chiralpak IG 5μ, 250×50; eluent A: hexane+0.1 vol % diethylamine; eluent B: ethanol; isocratic: 20% B; flow: 70 mL/min; temperature: 25° C.; UV: 280 nm

Analytical Chiral HPLC Method:

Instrument: Thermo Fisher UltiMate 3000; Column: Chiralpak IG 3μ, 100×4.6; eluent A: hexane+0.1 vol % diethylamine; eluent B: ethanol; isocratic: 20% B; flow: 1.4 mL/min; temperature: 25° C.; UV: 280 nm

Analytical chiral HPLC: Rₜ=3.19 min

The crude fraction was purified by preparative HPLC to give 13.0 mg (99% purity, 16% yield) of the title compound.

[α]²⁰_{D}: +36.6° (c=1.00, DMSO)

LC-MS (Method 1): Rₜ=1.03 min; MS (ESIpos): m/z=447 [M+H]⁺

¹H-NMR (400 MHz, DMSO-d6) δ [ppm]: 0.683 (6.44), 0.700 (6.54), 0.962 (6.22), 0.979 (6.40), 1.851 (0.75), 1.865 (0.86), 1.870 (0.87), 1.878 (0.80), 1.885 (0.77), 1.893 (0.89), 1.898 (0.95), 1.914 (0.72), 2.142 (0.50), 2.159 (0.69), 2.165 (0.57), 2.175 (0.55), 2.182 (0.71), 2.198 (0.48), 2.380 (0.78), 2.394 (0.79), 2.412 (1.37), 2.429 (0.66), 2.449 (0.70), 2.465 (1.68), 2.518 (1.73), 2.523 (1.19), 2.579 (2.01), 2.602 (2.79), 2.622 (0.99), 2.642 (0.44), 2.779 (0.52), 2.793 (2.62), 2.799 (1.32), 2.815 (2.45), 3.334 (16.00), 3.344 (3.17), 3.367 (2.38), 4.064 (1.86), 4.081 (3.82), 4.099 (1.80), 6.271 (9.09), 6.515 (4.53), 6.851 (1.26), 7.038 (1.21), 7.980 (2.60), 7.985 (2.61), 8.558 (2.39), 8.562 (2.36), 11.723 (1.02).

### Example 760

### 5-{(3R)-1-[1-(1H-imidazol-2-yl)-2-methylpropyl]-5′,6′-dihydrospiro[pyrrolidine-3,4′-pyrrolo[1,2-b]pyrazol]-2′-yl}-3-(trifluoromethyl)pyridin-2-amine (Diastereomer 2)

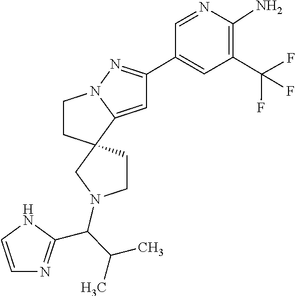 [SMILES: CC(C)C(c1ncc[nH]1)N1CC[C@@]2(CCn3nc(-c4cnc(N)c(C(F)(F)F)c4)cc32)C1]

For the preparation of the diastereomeric mixture of the title compound see Example 758. The diastereomers were separated by preparative chiral HPLC (method see Example 759) (diastereomer 2 Rₜ=10.03-13.90 min).

Analytical chiral HPLC (method see Example 759): Rₜ=6.44 min

The crude fraction was purified by preparative HPLC to give 14.0 mg (99% purity, 17% yield) of the title compound.

[α]²⁰_{D}: +27.8° (c=1.00, DMSO)

LC-MS (Method 1): Rₜ=1.03 min; MS (ESIpos): m/z=447 [M+H]⁺

¹H-NMR (400 MHz, DMSO-d6) δ [ppm]: 0.672 (4.91), 0.689 (5.00), 0.975 (4.85), 0.992 (4.97), 1.867 (1.06), 1.886 (2.06), 1.903 (1.14), 2.161 (0.52), 2.166 (0.44), 2.177 (0.43), 2.183 (0.54), 2.408 (0.46), 2.421 (0.62), 2.440 (0.87), 2.457 (0.64), 2.468 (0.76), 2.518 (1.77), 2.523 (1.12), 2.579 (0.74), 2.601 (0.89), 2.632 (0.77), 2.655 (2.24), 2.669 (2.64), 2.692 (0.74), 2.815 (0.83), 2.832 (0.51), 2.838 (0.65), 3.334 (16.00), 3.348 (2.09), 3.371 (1.86), 4.061 (0.90), 4.066 (0.87), 4.079 (1.72), 4.096 (0.88), 4.100 (0.89), 6.221 (7.58), 6.518 (3.50), 6.843 (0.84), 7.053 (0.81), 7.976 (2.02), 7.981 (2.03), 8.553 (1.87), 8.557 (1.82), 11.707 (0.83).

### Example 761

### 5-{(3S)-1-[1-(1H-imidazol-2-yl)propyl]-5′,6′-dihydrospiro[pyrrolidine-3,4′-pyrrolo[1,2-b]pyrazol]-2′-yl}-3-(trifluoromethyl)pyridin-2-amine (Diastereomer 1 and Diastereomer 2)

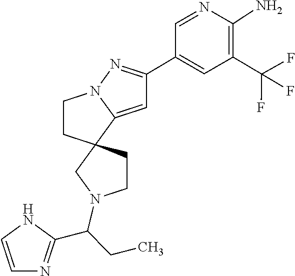 [SMILES: CCC(c1ncc[nH]1)N1CC[C@]2(CCn3nc(-c4cnc(N)c(C(F)(F)F)c4)cc32)C1]

5-[(3S)-5′,6′-Dihydrospiro[pyrrolidine-3,4-pyrrolo[1,2-b]pyrazol]-2′-yl]-3-(trifluoromethyl)pyridin-2-amine hydrogen chloride (1/1) (400 mg, 85% purity, 945 μmol) and 1-(1H-imidazol-2-yl)propan-1-one (176 mg, 1.42 mmol) were dissolved in methanol (10 mL). N,N-Diisopropylethylamine (660 μL, 3.8 mmol) and titanium(IV) isopropoxide (840 μL, 2.8 mmol) were added and the mixture was stirred for 3 h at 60° C. To the cold reaction mixture was added sodium cyanoborohydride (178 mg, 2.84 mmol) and stirred over night at 60° C. The cold reaction mixture was treated with water (2 mL) and stirred for 15 min. The suspension was filtered over celite and the filter cake was washed with methanol. The filtrate was concentrated. The crude product was purified by column chromatography (silica gel, hexane/ethyl acetate (0-100%) and dichloromethane/methanol (0-5%) gradient). 46 mg of the combined fractions were purified by preparative HPLC to give 23.0 mg (99% purity, 6% yield) of the title compound.

LC-MS (Method 1): Rₜ=0.93 min; MS (ESIpos): m/z=433 [M+H]⁺

¹H-NMR (400 MHz, DMSO-d6) δ [ppm]: 0.712 (5.21), 0.719 (5.18), 0.731 (12.53), 0.737 (11.24), 0.749 (5.81), 0.756 (4.97), 1.758 (0.56), 1.776 (1.00), 1.791 (1.67), 1.809 (2.26), 1.828 (2.36), 1.847 (2.49), 1.862 (2.08), 1.865 (2.13), 1.885 (1.89), 1.906 (3.91), 1.922 (5.72), 1.941 (3.32), 1.973 (0.42), 2.318 (0.47), 2.323 (1.04), 2.327 (1.53), 2.331 (1.02), 2.337 (0.45), 2.388 (0.49), 2.397 (0.51), 2.406 (1.20), 2.420 (1.43), 2.438 (2.14), 2.449 (1.79), 2.454 (1.47), 2.466 (2.19), 2.518 (6.47), 2.523 (4.94), 2.563 (4.19), 2.581 (4.20), 2.594 (1.90), 2.605 (5.62), 2.629 (0.90), 2.654 (3.72), 2.665 (1.40), 2.669 (1.84), 2.677 (2.60), 2.744 (0.80), 2.765 (1.64), 2.780 (1.40), 2.787 (1.15), 2.796 (3.52), 2.819 (2.94), 2.833 (0.76), 2.848 (1.19), 2.871 (1.22), 2.888 (0.54), 3.497 (1.72), 3.512 (2.54), 3.521 (2.88), 3.533 (2.11), 3.544 (1.47), 4.039 (0.52), 4.047 (1.48), 4.065 (4.62), 4.073 (2.72), 4.082 (6.57), 4.093 (2.11), 4.100 (2.99), 6.293 (13.25), 6.336 (16.00), 6.518 (13.52), 6.810 (5.89), 7.041 (3.85), 7.985 (4.26), 7.990 (7.70), 7.994 (4.68), 8.564 (3.76), 8.570 (6.77), 11.776 (3.19).

### Example 762

### 5-{(3S)-1-[1-(1H-imidazol-2-yl)propyl]-5′,6′-dihydrospiro[pyrrolidine-3,4′-pyrrolo[1,2-b]pyrazol]-2′-yl}-3-(trifluoromethyl)pyridin-2-amine (Diastereomer 1)

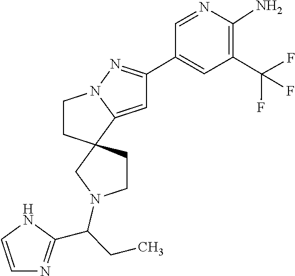 [SMILES: CCC(c1ncc[nH]1)N1CC[C@]2(CCn3nc(-c4cnc(N)c(C(F)(F)F)c4)cc32)C1]

The title compound from Example 761 was separated into diastereomers by preparative chiral HPLC (diastereomer 1 Rₜ=4.60-5.75 min).

Preparative Chiral HPLC Method:

Instrument: Sepiatec: Prep SFC100; Column: Chiral Art Amylos-SA 5μ, 250×50; eluent A: CO2; eluent B: 2-propanol+0.4 vol % diethylamine; isocratic: 20% B; flow: 100 mL/min; temperature: 40° C.; BPR: 150 bar; UV: 265 nm

Analytical Chiral HPLC Method:

Instrument: Waters Acquity UPC2 QDA; Column: Chiral Art Amylose-SA 3μ, 100×4.6; eluent A: CO2; eluent B: 2-propanol+0.4 vol % diethylamine; isocratic: 20% B; flow: 4 mL/min; temperature: 40° C.; BPR: 1800 psi; UV: 254 nm

Analytical chiral HPLC: Rₜ=2.75 min

The crude fraction was purified by preparative HPLC to give 25.0 mg (99% purity, 17% yield) of the title compound.

[α]²⁰_{D}: −34.9° (c=1.00, DMSO)

LC-MS (Method 1): Rₜ=0.93 min; MS (ESIpos): m/z=433 [M+H]⁺

¹H-NMR (400 MHz, DMSO-d6) δ [ppm]: 0.713 (5.73), 0.731 (13.41), 0.749 (6.14), 1.758 (0.52), 1.777 (0.77), 1.792 (1.38), 1.813 (1.78), 1.829 (1.90), 1.844 (1.77), 1.862 (1.97), 1.886 (1.82), 1.906 (3.15), 1.922 (3.02), 1.941 (1.64), 1.953 (0.63), 1.973 (0.45), 2.388 (0.64), 2.406 (1.44), 2.420 (1.72), 2.438 (2.46), 2.455 (1.61), 2.477 (2.78), 2.518 (5.71), 2.523 (4.47), 2.564 (4.36), 2.593 (1.80), 2.611 (1.99), 2.630 (0.92), 2.745 (0.96), 2.766 (2.06), 2.782 (1.83), 2.788 (1.58), 2.797 (4.33), 2.820 (3.46), 3.499 (2.01), 3.513 (2.56), 3.521 (2.18), 3.535 (1.92), 4.065 (3.44), 4.083 (6.48), 4.100 (3.34), 6.336 (16.00), 6.516 (9.43), 6.927 (0.61), 7.989 (5.26), 7.995 (5.34), 8.570 (4.95), 8.574 (4.91), 11.782 (0.68).

### Example 763

### 5-{(3S)-1-[1-(1H-imidazol-2-yl)propyl]-5′,6′-dihydrospiro[pyrrolidine-3,4′-pyrrolo[1,2-b]pyrazol]-2′-yl}-3-(trifluoromethyl)pyridin-2-amine (Diastereomer 2)

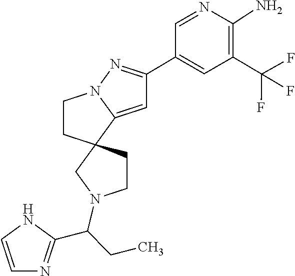 [SMILES: CCC(c1ncc[nH]1)N1CC[C@]2(CCn3nc(-c4cnc(N)c(C(F)(F)F)c4)cc32)C1]

For the preparation of the diastereomeric mixture of the title compound see Example 761. The diastereomers were separated by preparative chiral HPLC (method see Example 762) (diastereomer 2 Rₜ=6.28-7.60 min).

Analytical chiral HPLC (method see Example 762): Rₜ=4.53 min

The crude fraction was purified by preparative HPLC to give 22.0 mg (99% purity, 15% yield) of the title compound.

[α]²⁰_{D}: −55.8° (c=1.00, DMSO)

LC-MS (Method 1): Rₜ=0.93 min; MS (ESIpos): m/z=433 [M+H]⁺

¹H-NMR (400 MHz, DMSO-d6) δ [ppm]: 0.719 (5.13), 0.738 (12.15), 0.755 (5.42), 1.752 (0.57), 1.770 (0.68), 1.786 (1.19), 1.808 (1.36), 1.827 (1.43), 1.831 (1.27), 1.846 (1.54), 1.865 (1.65), 1.884 (0.91), 1.904 (2.10), 1.922 (4.52), 1.941 (2.59), 2.322 (0.72), 2.326 (1.03), 2.331 (0.73), 2.397 (0.48), 2.416 (0.89), 2.429 (1.54), 2.449 (1.95), 2.466 (2.54), 2.518 (5.09), 2.522 (3.29), 2.562 (0.90), 2.581 (4.75), 2.604 (6.16), 2.622 (0.81), 2.654 (4.48), 2.664 (1.08), 2.668 (1.32), 2.677 (2.96), 2.833 (0.80), 2.848 (1.46), 2.871 (1.50), 2.888 (0.65), 3.508 (1.79), 3.521 (2.30), 3.530 (1.93), 3.544 (1.71), 4.038 (0.57), 4.047 (1.73), 4.062 (2.67), 4.066 (2.67), 4.073 (2.45), 4.078 (2.48), 4.092 (1.66), 4.100 (0.55), 4.104 (0.51), 6.292 (16.00), 6.516 (8.16), 6.808 (3.04), 7.045 (2.76), 7.984 (4.57), 7.989 (4.60), 8.563 (4.28), 8.567 (4.13), 11.778 (1.85).

### Example 764

### 5-{(3′R)-1′-[1-(1H-imidazol-2-yl)propyl]-6,7-dihydrospiro[pyrazolo[5,1-c][1,4]oxazine-4,3′-pyrrolidin]-2-yl}-3-(trifluoromethyl)pyridin-2-amine (Diastereomer 1 and Diastereomer 2)

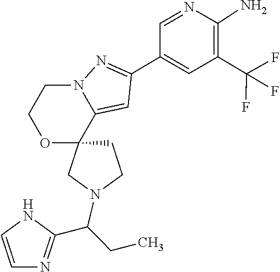 [SMILES: CCC(c1ncc[nH]1)N1CC[C@]2(C1)OCCn1nc(-c3cnc(N)c(C(F)(F)F)c3)cc12]

The compound was prepared similarly to Example 761 using 5-[(3′R)-6,7-dihydrospiro[pyrazolo[5,1-c][1,4]oxazine-4,3′-pyrrolidin]-2-yl]-3-(trifluoromethyl)pyridin-2-amine hydrogen chloride (1/1) (475 mg, 70% purity, 885 μmol) and 1-(1H-imidazol-2-yl)propan-1-one (165 mg, 1.33 mmol) to give 144 mg (36% yield). 34 mg were purified by HPLC to yield 18.0 mg (99% purity, 5% yield) of the title compound.

LC-MS (Method 1): Rₜ=0.90 min; MS (ESIpos): m/z=449 [M+H]⁺

¹H-NMR (400 MHz, DMSO-d6) δ [ppm]: 0.683 (5.64), 0.691 (4.40), 0.702 (13.52), 0.710 (9.13), 0.720 (6.32), 0.728 (3.95), 1.748 (0.58), 1.767 (0.91), 1.781 (1.38), 1.790 (1.12), 1.800 (1.80), 1.823 (2.00), 1.836 (1.82), 1.850 (2.13), 1.868 (2.35), 1.887 (1.14), 1.902 (0.66), 2.029 (0.73), 2.038 (1.21), 2.043 (1.10), 2.052 (1.47), 2.061 (1.55), 2.071 (2.43), 2.090 (2.03), 2.103 (1.90), 2.121 (2.17), 2.134 (1.32), 2.152 (0.76), 2.518 (5.58), 2.523 (3.87), 2.575 (2.07), 2.597 (2.31), 2.696 (3.46), 2.721 (3.82), 2.773 (2.11), 2.791 (1.72), 2.799 (3.16), 2.811 (1.36), 2.830 (1.32), 2.852 (1.02), 2.879 (2.80), 2.905 (1.86), 3.031 (3.44), 3.057 (2.99), 3.472 (2.33), 3.485 (2.82), 3.495 (2.40), 3.509 (2.15), 3.951 (0.83), 3.965 (2.17), 3.978 (3.65), 3.990 (5.14), 4.004 (3.84), 4.048 (5.00), 4.057 (5.78), 4.062 (5.22), 6.483 (10.69), 6.523 (16.00), 6.575 (12.21), 6.801 (5.59), 6.804 (4.95), 6.808 (3.99), 7.034 (4.22), 7.052 (2.72), 8.018 (7.05), 8.593 (3.28), 8.599 (6.96), 8.603 (4.65), 11.833 (3.53).

### Example 765

### 5-{(3′R)-1′-[1-(1H-imidazol-2-yl)propyl]-6,7-dihydrospiro[pyrazolo[5,1-c][1,4]oxazine-4,3′-pyrrolidin]-2-yl}-3-(trifluoromethyl)pyridin-2-amine (Diastereomer 1)

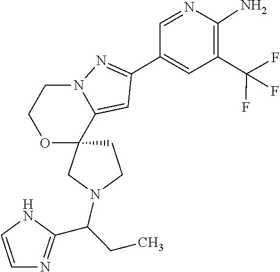 [SMILES: CCC(c1ncc[nH]1)N1CC[C@]2(C1)OCCn1nc(-c3cnc(N)c(C(F)(F)F)c3)cc12]

The title compound from Example 764 was separated into diastereomers by preparative chiral HPLC (diastereomer 1 Rₜ=3.03-3.79 min).

Preparative Chiral HPLC Method:

Instrument: Sepiatec: Prep SFC100; Column: Chiral Art Amylos-SA 5μ, 250×50; eluent A: CO2; eluent B: 2-propanol+0.4 vol % diethylamine; isocratic: 30% B; flow: 100 mL/min; temperature: 40° C.; BPR: 150 bar; UV: 265 nm

Analytical Chiral HPLC Method:

Instrument: Waters Acquity UPC2 QDA; Column: Chiral Art Amylose-SA 3μ, 100×4.6; eluent A: CO2; eluent B: 2-propanol+0.4 vol % diethylamine; isocratic: 30% B; flow: 4 mL/min; temperature: 40° C.; BPR: 1800 psi; UV: 254 nm

Analytical chiral HPLC: Rₜ=0.86 min

The crude fraction was purified by preparative HPLC to give 20.0 mg (99% purity, 18% yield) of the title compound.

[α]²⁰_{D}: +63.2° (c=1.00, DMSO)

LC-MS (Method 1): Rₜ=0.90 min; MS (ESIpos): m/z=449 [M+H]⁺

¹H-NMR (400 MHz, DMSO-d6) δ [ppm]: 0.691 (5.04), 0.710 (12.02), 0.728 (5.31), 1.767 (0.41), 1.786 (0.60), 1.791 (0.52), 1.801 (1.18), 1.819 (1.32), 1.824 (1.42), 1.835 (1.32), 1.843 (1.34), 1.848 (1.45), 1.853 (1.25), 1.867 (1.54), 1.886 (0.71), 2.011 (0.45), 2.030 (0.88), 2.043 (1.22), 2.061 (1.70), 2.074 (0.55), 2.080 (0.96), 2.100 (0.82), 2.118 (1.43), 2.133 (1.47), 2.148 (0.71), 2.166 (0.46), 2.518 (2.93), 2.523 (2.02), 2.569 (0.68), 2.589 (1.36), 2.604 (1.39), 2.624 (0.67), 2.773 (2.51), 2.799 (3.57), 2.812 (0.88), 2.830 (1.77), 2.852 (1.50), 2.870 (0.80), 2.880 (4.15), 2.905 (2.78), 3.469 (1.76), 3.483 (2.27), 3.493 (1.80), 3.506 (1.70), 3.933 (0.44), 3.951 (1.15), 3.965 (2.94), 3.978 (3.30), 3.990 (2.01), 4.012 (0.55), 4.019 (0.63), 4.043 (3.09), 4.054 (5.05), 4.068 (1.79), 6.483 (16.00), 6.574 (7.66), 6.810 (1.70), 7.052 (1.63), 8.015 (4.50), 8.020 (4.60), 8.593 (4.21), 8.597 (4.21), 11.837 (1.43).

### Example 766

### 5-{(3′R)-1′-[1-(1H-imidazol-2-yl)propyl]-6,7-dihydrospiro[pyrazolo[5,1-c][1,4]oxazine-4,3′-pyrrolidin]-2-yl}-3-(trifluoromethyl)pyridin-2-amine (Diastereomer 2)

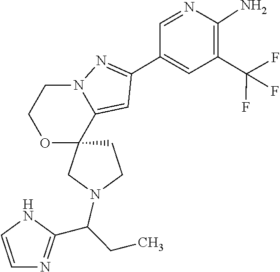 [SMILES: CCC(c1ncc[nH]1)N1CC[C@]2(C1)OCCn1nc(-c3cnc(N)c(C(F)(F)F)c3)cc12]

For the preparation of the diastereomeric mixture of the title compound see Example 764. The diastereomers were separated by preparative chiral HPLC (method see Example 765) (diastereomer 2 Rₜ=4.43-5.62 min).

Analytical chiral HPLC (method see Example 765): Rₜ=1.70 min

The crude fraction was purified by preparative HPLC (basic method) to give 30.0 mg (99% purity, 27% yield) of the title compound.

[α]²⁰_{D}: +43.9° (c=1.00, DMSO)

LC-MS (Method 1): Rₜ=0.90 min; MS (ESIpos): m/z=449 [M+H]⁺

¹H-NMR (400 MHz, DMSO-d6) δ [ppm]: 0.684 (5.19), 0.702 (12.43), 0.720 (5.46), 1.748 (0.50), 1.767 (0.67), 1.772 (0.62), 1.781 (1.10), 1.789 (0.75), 1.799 (1.05), 1.805 (1.17), 1.823 (1.07), 1.837 (1.03), 1.850 (1.24), 1.855 (1.08), 1.869 (1.48), 1.888 (0.76), 1.902 (0.44), 2.038 (0.94), 2.052 (1.19), 2.071 (2.18), 2.090 (1.88), 2.107 (1.53), 2.121 (1.48), 2.137 (0.60), 2.154 (0.40), 2.518 (2.75), 2.523 (2.08), 2.575 (1.75), 2.597 (1.88), 2.615 (0.67), 2.696 (3.37), 2.722 (3.75), 2.770 (0.78), 2.784 (1.11), 2.789 (1.39), 2.805 (1.32), 2.825 (0.58), 3.031 (3.39), 3.056 (2.92), 3.473 (1.80), 3.487 (2.21), 3.497 (1.87), 3.510 (1.72), 3.991 (3.81), 4.004 (3.60), 4.048 (3.72), 4.058 (3.55), 4.062 (4.12), 4.074 (1.69), 6.523 (16.00), 6.571 (7.70), 6.801 (3.81), 7.034 (3.30), 8.018 (4.67), 8.023 (4.68), 8.599 (4.36), 8.603 (4.27), 11.832 (2.07).

### Example 767

### {(3R)-2′-[6-amino-5-(trifluoromethyl)pyridin-3-yl]-5′,6′-dihydrospiro[pyrrolidine-3,4′-pyrrolo[1,2-b]pyrazol]-1-yl}(1H-imidazol-2-yl)methanone

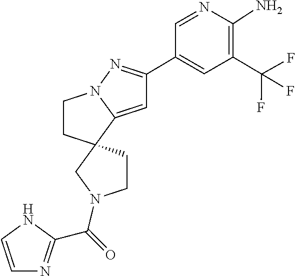 [SMILES: Nc1ncc(-c2cc3n(n2)CC[C@@]32CCN(C(=O)c3ncc[nH]3)C2)cc1C(F)(F)F]

1H-Imidazole-2-carboxylic acid (128 mg, 1.14 mmol) was dissolved in DMF (6 mL). N,N-Diisopropylethylamine (500 μL, 2.8 mmol) and HATU (650 mg, 1.71 mmol) were added and the mixture was stirred for 30 min at room temperature. 5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (250 mg, 82% purity, 570 μmol) was added and stirred over the weekend at room temperature. 1H-Imidazole-2-carboxylic acid (128 mg, 1.14 mmol) and HATU (433 mg, 1.14 mmol) were added again and the mixture was stirred for 5 h at 60° C. The reaction mixture was allowed to cool down, diluted with water and brine. The aqueous phase was extracted with ethyl acetate. The combined organic layers were dried over sodium sulfate, filtered and concentrated. The residue was purified by column chromatography (silica gel, dichloromethane/methanol (0-5%) gradient) followed by preparative HPLC to give 28.0 mg (98% purity, 12% yield) of the title compound.

LC-MS (Method 1): Rₜ=0.87 min; MS (ESIpos): m/z=419 [M+H]⁺

¹H-NMR (400 MHz, DMSO-d6) δ [ppm]: 2.074 (3.84), 2.097 (1.21), 2.116 (2.35), 2.133 (1.15), 2.181 (1.22), 2.198 (2.60), 2.216 (1.23), 2.318 (0.99), 2.323 (2.26), 2.327 (3.17), 2.331 (2.23), 2.337 (0.99), 2.518 (12.18), 2.523 (8.68), 2.567 (0.82), 2.582 (1.34), 2.600 (3.68), 2.617 (4.12), 2.633 (2.76), 2.649 (1.31), 2.660 (1.25), 2.665 (2.77), 2.669 (3.39), 2.673 (2.24), 2.678 (1.05), 3.705 (0.97), 3.735 (5.04), 3.754 (0.61), 3.799 (0.58), 3.816 (1.24), 3.830 (0.84), 3.846 (0.75), 4.189 (2.49), 4.207 (6.64), 4.224 (3.42), 4.238 (3.75), 4.260 (3.64), 4.277 (0.77), 4.290 (1.74), 4.294 (1.81), 4.313 (1.67), 4.329 (0.68), 6.525 (16.00), 6.530 (9.87), 6.539 (10.40), 7.046 (4.49), 7.048 (4.52), 7.132 (3.20), 7.253 (3.86), 7.282 (2.76), 8.021 (3.36), 8.027 (4.71), 8.033 (2.45), 8.589 (3.07), 8.595 (3.77), 8.602 (2.18), 12.965 (1.24).

### Example 768

### {(3R)-2′-[6-amino-5-(trifluoromethyl)pyridin-3-yl]-5′,6′-dihydrospiro[pyrrolidine-3,4′-pyrrolo[1,2-b]pyrazol]-1-yl}(1-methyl-1H-imidazol-2-yl)methanone

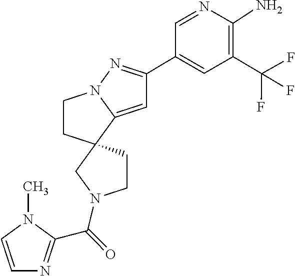 [SMILES: Cn1ccnc1C(=O)N1CC[C@@]2(CCn3nc(-c4cnc(N)c(C(F)(F)F)c4)cc32)C1]

1-Methyl-1H-imidazole-2-carboxylic acid (65.7 mg, 521 μmol) was dissolved in DMF (3.5 mL). N,N-Diisopropylethylamine (240 μL, 1.4 mmol) and HATU (264 mg, 695 μmol) were added and the mixture was stirred for 30 min at room temperature. 5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (125 mg, 347 μmol) was added and stirred for 1 h at room temperature. The reaction mixture was diluted with water and extracted twice with ethyl acetate. The combined organic layers were dried over sodium sulfate, filtered and concentrated. The residue was purified by preparative HPLC to give 21.0 mg (99% purity, 14% yield) of the title compound.

LC-MS (Method 1): Rₜ=0.89 min; MS (ESIpos): m/z=433 [M+H]⁺

¹H NMR (400 MHz, DMSO-d₆) δ ppm: 2.08-2.19 (m, 2H), 2.52-2.65 (m, 2H), 3.65-3.84 (m, 2H), 3.87 (s, 3H), 4.01-4.27 (m, 4H), 6.52-6.55 (m, 3H) 6.96 and 7.04 (2d, 1H) 7.31 and 7.35 (2d, 1H), 8.01-8.04 (m, 1H), 8.58-8.61 (m, 1H).

### Example 769

### 3-[(1R)-1-(2,6-dichloro-3-fluorophenyl)ethoxy]-5-{(3R)-1-[(1H-imidazol-2-yl)methyl]-5′,6′-dihydrospiro[pyrrolidine-3,4′-pyrrolo[1,2-b]pyrazol]-2′-yl}pyridin-2-amine

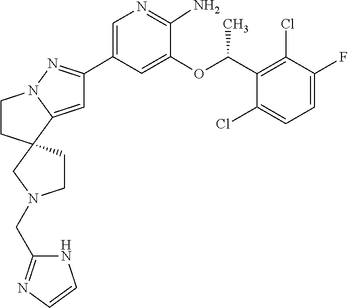 [SMILES: C[C@@H](Oc1cc(-c2cc3n(n2)CC[C@@]32CCN(Cc3ncc[nH]3)C2)cnc1N)c1c(Cl)ccc(F)c1Cl]

3-[(1R)-1-(2,6-Dichloro-3-fluorophenyl)ethoxy]-5-[(3R)-5′,6′-dihydrospiro[pyrrolidine-3,4′-pyrrolo[1,2-b]pyrazol]-2′-yl]pyridin-2-amine hydrogen chloride (1/1) (270 mg, 541 μmol) and 1H-imidazole-2-carbaldehyde (78.0 mg, 812 μmol) were dissolved in THF (5.5 mL). Acetic acid (46 μL) was added and the mixture was stirred for 30 min at room temperature. Sodium triacetoxyborohydride (229 mg, 1.08 mmol) was added and stirred over night at room temperature. The reaction mixture was diluted with water and extracted with ethyl acetate. The combined organic layers were washed with brine, dried over sodium sulfate, filtered and concentrated. The residue was purified by preparative HPLC. The fraction was dissolved in dichloromethane and basified with 1M sodium hydroxide solution. The organic phase was concentrated to give 5.50 mg (95% purity, 2% yield) of the title compound.

LC-MS (Method 1): Rₜ=1.06 min; MS (ESIpos): m/z=542 [M+H]⁺

¹H NMR (400 MHz, DMSO-d₆) δ ppm: 8.16 (s, 1H), 7.88 (d, 1H), 7.55 (dd, 1H), 7.40-7.47 (m, 1H), 7.12 (d, 1H), 6.91 (br s, 2H), 6.17 (s, 1H), 6.05 (q, 1H), 3.98-4.09 (m, 2H), 3.67 (s, 2H), 2.53-2.82 (m, 5H), 2.41-2.47 (m, 1H), 1.92-2.04 (m, 2H), 1.78 (d, 3H).

### Example 770

### 5-{(3R)-1-[1-(1H-imidazol-2-yl)ethyl]-5′,6′-dihydrospiro[pyrrolidine-3,4′-pyrrolo[1,2-b]pyrazol]-2′-yl}-3-(trifluoromethyl)pyridin-2-amine (Diastereomer 1)

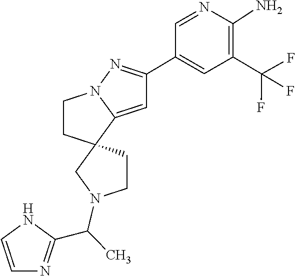 [SMILES: CC(c1ncc[nH]1)N1CC[C@@]2(CCn3nc(-c4cnc(N)c(C(F)(F)F)c4)cc32)C1]

The diastereomeric mixture of compound 5-{(3R)-1-[1-(1H-imidazol-2-yl)ethyl]-5′,6′-dihydrospiro[pyrrolidine-3,4′-pyrrolo[1,2-b]pyrazol]-2′-yl}-3-(trifluoromethyl)pyridin-2-amine (35.0 mg, 83.8 μmol) was separated into diastereomers by preparative chiral HPLC to give 14.0 mg (90% purity, 36% yield) of the title compound (diastereomer 1 Rₜ=7.1-8.9 min).

Preparative Chiral HPLC Method:

Instrument: PrepCon Labomatic HPLC-1; Column: Chiralpak IG 5μ, 250×50; eluent A: hexane+0.1 vol % diethylamine; eluent B: ethanol; isocratic: 30% B; flow: 50 mL/min; temperature: 25° C.; UV: 254 nm

Analytical Chiral HPLC Method:

Instrument: Thermo Fisher UltiMate 3000; Column: Chiralpak IG 3μ, 100×4.6; eluent A: hexane+0.1 vol % diethylamine; eluent B: ethanol; isocratic: 30% B; flow: 1.4 mL/min; temperature: 25° C.; UV: 254 nm

Analytical chiral HPLC: Rₜ=2.65 min

LC-MS (Method 1): Rₜ=0.87 min; MS (ESIpos): m/z=419 [M+H]⁺

¹H-NMR (400 MHz, DMSO-d6) δ [ppm]: 0.776 (0.41), 0.794 (0.99), 0.813 (0.61), 0.819 (0.43), 0.836 (0.51), 0.861 (0.54), 1.005 (0.89), 1.034 (0.76), 1.084 (1.64), 1.204 (0.50), 1.231 (1.42), 1.259 (3.24), 1.367 (11.57), 1.384 (11.92), 1.907 (0.71), 1.919 (1.33), 1.939 (2.35), 1.955 (2.46), 1.960 (2.01), 1.976 (1.48), 1.987 (0.55), 1.993 (0.57), 2.007 (0.43), 2.414 (0.69), 2.432 (1.32), 2.446 (1.55), 2.465 (2.10), 2.481 (1.94), 2.518 (2.07), 2.523 (1.78), 2.530 (4.98), 2.546 (1.92), 2.554 (4.06), 2.560 (1.78), 2.577 (1.26), 2.591 (1.10), 2.598 (1.41), 2.614 (1.68), 2.633 (0.81), 2.717 (0.92), 2.737 (1.80), 2.754 (1.45), 2.760 (1.06), 2.776 (0.68), 2.794 (3.74), 2.817 (3.19), 3.675 (0.85), 3.692 (3.36), 3.709 (3.31), 3.726 (0.82), 4.071 (2.64), 4.088 (5.06), 4.107 (2.60), 6.399 (16.00), 6.519 (8.07), 6.900 (0.88), 7.996 (4.50), 8.002 (4.57), 8.579 (4.23), 8.583 (4.12), 10.856 (0.45), 11.771 (0.49).

### Example 771

### 5-{(3R)-1-[1-(1H-imidazol-2-yl)ethyl]-5′,6′-dihydrospiro[pyrrolidine-3,4′-pyrrolo[1,2-b]pyrazol]-2′-yl}-3-(trifluoromethyl)pyridin-2-amine (Diastereomer 2)

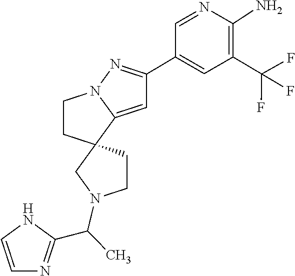 [SMILES: CC(c1ncc[nH]1)N1CC[C@@]2(CCn3nc(-c4cnc(N)c(C(F)(F)F)c4)cc32)C1]

The diastereomeric mixture of compound 5-{(3R)-1-[1-(1H-imidazol-2-yl)ethyl]-5′,6′-dihydrospiro[pyrrolidine-3,4′-pyrrolo[1,2-b]pyrazol]-2′-yl}-3-(trifluoromethyl)pyridin-2-amine (35.0 mg, 83.8 μmol) was separated into diastereomers by preparative chiral HPLC (method see Example 770) to give 3.00 mg (90% purity, 8% yield) of the title compound (diastereomer 2 Rₜ=14.4-18.9 min).

Analytical chiral HPLC (method see Example 770): Rₜ=4.38 min

LC-MS (Method 1): Rₜ=0.87 min; MS (ESIpos): m/z=419 [M+H]⁺

¹H-NMR (400 MHz, DMSO-d6) δ [ppm]: 0.776 (1.31), 0.795 (2.80), 0.813 (1.82), 0.819 (1.28), 0.836 (1.39), 0.843 (1.08), 0.855 (0.76), 0.862 (1.56), 0.871 (0.56), 0.879 (0.69), 0.886 (0.42), 0.904 (0.55), 0.916 (0.51), 0.933 (0.99), 0.951 (0.53), 0.973 (0.60), 1.005 (2.64), 1.012 (0.49), 1.028 (0.52), 1.034 (2.02), 1.049 (1.10), 1.084 (4.80), 1.100 (0.69), 1.142 (0.46), 1.173 (0.46), 1.204 (2.16), 1.232 (2.65), 1.259 (7.90), 1.270 (0.87), 1.278 (0.64), 1.286 (0.82), 1.295 (0.54), 1.303 (0.63), 1.316 (0.45), 1.352 (2.26), 1.366 (11.94), 1.383 (12.22), 1.405 (0.94), 1.421 (0.88), 1.425 (0.86), 1.440 (0.66), 1.444 (0.65), 1.489 (0.40), 1.527 (0.67), 1.543 (0.58), 1.572 (0.41), 1.592 (0.44), 1.609 (0.43), 1.758 (0.43), 1.774 (0.42), 1.788 (0.48), 1.906 (0.60), 1.922 (0.55), 1.936 (1.64), 1.943 (1.77), 1.953 (3.39), 1.963 (2.43), 1.972 (1.96), 1.977 (2.00), 2.323 (0.74), 2.327 (0.98), 2.332 (0.77), 2.418 (0.78), 2.437 (1.26), 2.450 (1.90), 2.466 (2.39), 2.470 (2.68), 2.518 (3.84), 2.523 (2.24), 2.530 (1.65), 2.543 (0.84), 2.548 (0.70), 2.564 (1.12), 2.576 (3.04), 2.584 (2.12), 2.599 (4.99), 2.625 (0.88), 2.652 (4.70), 2.665 (0.93), 2.669 (1.32), 2.675 (3.27), 2.824 (0.86), 2.843 (1.37), 2.861 (1.45), 2.879 (0.70), 3.691 (0.90), 3.708 (3.42), 3.725 (3.37), 3.742 (0.84), 4.021 (0.43), 4.040 (0.67), 4.048 (1.75), 4.063 (3.28), 4.067 (2.69), 4.077 (2.53), 4.082 (3.28), 4.097 (1.80), 4.104 (0.98), 4.123 (0.68), 5.758 (0.41), 6.370 (16.00), 6.435 (0.53), 6.518 (8.58), 6.793 (0.46), 6.997 (0.43), 7.994 (4.52), 7.999 (4.75), 8.575 (4.32), 8.579 (4.40), 10.855 (0.98), 11.780 (0.65).

### Example 772

### (3R)-2′-[6-amino-5-(trifluoromethyl)pyridin-3-yl]-N-[1-(3-chloropyridin-4-yl)cyclobutyl]-5′,6′-dihydrospiro[pyrrolidine-3,4′-pyrrolo[1,2-b]pyrazole]-1-carboxamide

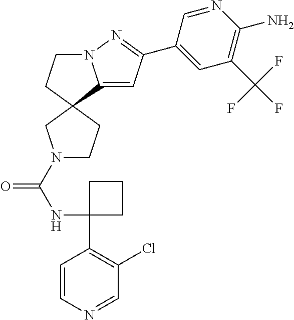 [SMILES: Nc1ncc(-c2cc3n(n2)CC[C@@]32CCN(C(=O)NC3(c4ccncc4Cl)CCC3)C2)cc1C(F)(F)F]

1-(3-Chloropyridin-4-yl)cyclobutan-1-amine (24.4 mg, 133 μmol), CDI (19.8 mg, 122 μmol) and N,N-diisopropylethylamine (120 μL, 670 μmol) were dissolved in DMF (1.5 mL) and stirred for 1 h at room temperature. A solution of 5-[(3R)-5′,6′-dihydrospiro[pyrrolidine-3,4′-pyrrolo[1,2-b]pyrazol]-2′-yl]-3-(trifluoromethyl)pyridin-2-amine hydrogen chloride (1/1) (50.0 mg, 80% purity, 111 μmol) in DMF was added and the mixture was stirred over night at 60° C. The reaction mixture was allowed to cool down, concentrated, treated with toluene and evaporated again. The residue was purified by preparative HPLC to give 2.80 mg (92% purity, 4% yield) of the title compound.

LC-MS (Method 1): Rₜ=1.07 min; MS (ESIpos): m/z=532 [M+H]⁺

¹H-NMR (400 MHz, CHLOROFORM-d) δ [ppm]: 0.812 (0.64), 0.989 (0.77), 1.005 (0.91), 1.110 (0.47), 1.185 (1.29), 1.752 (0.70), 1.762 (1.00), 1.774 (1.55), 1.779 (1.15), 1.790 (1.41), 1.797 (1.25), 1.802 (1.79), 1.814 (1.19), 1.824 (0.88), 1.837 (0.45), 1.978 (0.77), 1.997 (1.17), 2.010 (1.86), 2.025 (1.65), 2.041 (1.10), 2.088 (0.45), 2.109 (1.30), 2.130 (2.99), 2.150 (3.92), 2.157 (2.67), 2.170 (1.89), 2.179 (2.51), 2.199 (1.20), 2.450 (0.80), 2.467 (1.84), 2.483 (2.59), 2.500 (4.33), 2.516 (4.07), 2.526 (4.12), 2.531 (4.23), 2.536 (4.55), 2.549 (9.25), 2.557 (2.73), 2.567 (3.23), 2.581 (2.75), 2.598 (3.79), 2.632 (2.24), 2.645 (1.71), 2.665 (0.73), 2.975 (0.41), 3.035 (0.48), 3.374 (3.32), 3.398 (6.50), 3.425 (2.57), 3.431 (2.14), 3.443 (9.12), 3.449 (4.20), 3.467 (5.40), 3.516 (1.49), 3.529 (1.78), 3.536 (2.18), 3.549 (1.84), 3.560 (1.27), 3.574 (0.97), 4.172 (6.15), 4.189 (11.35), 4.207 (5.63), 4.929 (9.76), 5.150 (5.21), 6.086 (14.39), 6.116 (1.12), 6.929 (0.69), 7.451 (0.79), 7.465 (7.47), 7.477 (7.59), 8.021 (6.01), 8.026 (6.16), 8.044 (0.45), 8.404 (10.35), 8.417 (10.03), 8.427 (16.00), 8.510 (5.84), 8.514 (5.82).

### Example 773

### (3R)-2′-[6-amino-5-(trifluoromethyl)pyridin-3-yl]-N-[2-(3-chloropyridin-4-yl)propan-2-yl]-5′,6′-dihydrospiro[pyrrolidine-3,4′-pyrrolo[1,2-b]pyrazole]-1-carboxamide

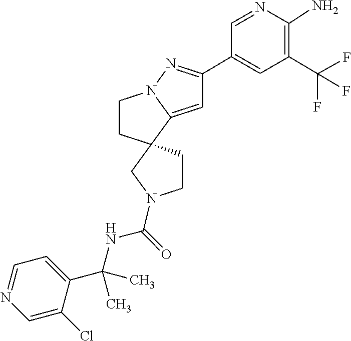 [SMILES: CC(C)(NC(=O)N1CC[C@@]2(CCn3nc(-c4cnc(N)c(C(F)(F)F)c4)cc32)C1)c1ccncc1Cl]

2-(3-Chloropyridin-4-yl)propan-2-amine (41.7 mg, 245 μmol), CDI (43.3 mg, 267 μmol) and N,N-diisopropylethylamine (150 μL, 890 μmol) were dissolved in DMF (1 mL) and stirred for 1 h at rt. A solution of 5-[(3R)-5′,6′-dihydrospiro[pyrrolidine-3,4′-pyrrolo[1,2-b]pyrazol]-2′-yl]-3-(trifluoromethyl)pyridin-2-amine hydrogen chloride (1/1) (100 mg, 80% purity, 222 μmol) in DMF was added dropwise and the mixture was stirred over night at rt. The reaction mixture was poured into water and extracted three times with ethyl acetate. The combined organic layers were dried over a hydrophobic filter and concentrated. The residue was purified by column chromatography (silica gel, dichloromethane/methanol (0-8%) gradient). The crude fraction was purified by preparative HPLC. The residue was dissolved in ethyl acetate and basified with a sodium bicarbonate solution. The aqueous phase was extracted three times with ethyl acetate. The combined organic layers were dried over a hydrophobic filter and concentrated to give 18.8 mg (90% purity, 15% yield) of the title compound.

LC-MS (Method 1): Rₜ=1.03 min; MS (ESIpos): m/z=520 [M+H]⁺

¹H NMR (400 MHz, CHLOROFORM-d) δ ppm: 0.009 (9.69), 1.192 (1.39), 1.567 (16.00), 1.620 (10.62), 1.738 (10.60), 1.742 (10.49), 2.046 (0.60), 2.060 (0.55), 2.166 (0.45), 2.185 (0.74), 2.202 (0.48), 2.214 (0.46), 2.492 (0.52), 2.507 (0.71), 2.526 (0.86), 2.542 (0.59), 2.564 (0.63), 2.581 (0.98), 2.584 (0.87), 2.600 (0.86), 2.633 (0.47), 3.415 (1.03), 3.440 (2.23), 3.476 (2.81), 3.488 (0.47), 3.495 (1.04), 3.500 (1.30), 3.512 (0.48), 3.541 (0.50), 3.556 (0.60), 3.561 (0.73), 3.566 (0.45), 3.576 (0.58), 3.580 (0.46), 4.192 (1.36), 4.211 (2.64), 4.227 (1.30), 4.229 (1.37), 4.753 (0.55), 4.843 (1.82), 4.944 (2.75), 6.150 (7.43), 6.176 (1.04), 7.218 (0.94), 7.232 (0.92), 7.349 (2.36), 7.361 (2.37), 7.432 (0.85), 7.445 (0.89), 8.045 (1.82), 8.050 (1.76), 8.276 (1.32), 8.290 (1.35), 8.364 (1.30), 8.380 (3.99), 8.393 (4.41), 8.432 (5.63), 8.459 (1.64), 8.539 (1.96), 8.542 (1.89).

### Example 774

### (3R)-2′-[6-amino-5-(trifluoromethyl)pyridin-3-yl]-N-[1-(pyridin-4-yl)cyclobutyl]-5′,6′-dihydrospiro[pyrrolidine-3,4′-pyrrolo[1,2-b]pyrazole]-1-carboxamide

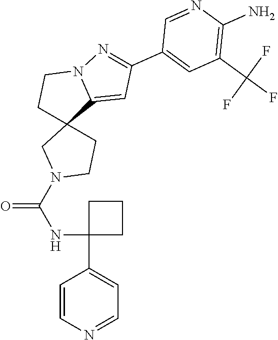 [SMILES: Nc1ncc(-c2cc3n(n2)CC[C@@]32CCN(C(=O)NC3(c4ccncc4)CCC3)C2)cc1C(F)(F)F]

1-(Pyridin-4-yl)cyclobutan-1-amine (19.8 mg, 133 μmol), CDI (19.8 mg, 122 μmol) and N,N-diisopropylethylamine (120 μL, 670 μmol) were dissolved in DMF (1.5 mL) and stirred for 1 h at rt. A solution of 5-[(3R)-5′,6′-dihydrospiro[pyrrolidine-3,4′-pyrrolo[1,2-b]pyrazol]-2′-yl]-3-(trifluoromethyl)pyridin-2-amine hydrogen chloride (1/1) (50.0 mg, 80% purity, 111 μmol) in DMF was added and the mixture was stirred for 2 h at 60° C. and 7 h at 100° C. The reaction mixture was allowed to cool down, concentrated, treated with toluene and evaporated again. The residue was purified by preparative HPLC followed by preparative TLC (dichloromethane/methanol 9:1) to give 5.80 mg (96% purity, 10% yield) of the title compound.

LC-MS (Method 1): Rₜ=0.97 min; MS (ESIpos): m/z=498 [M+H]⁺

¹H-NMR (400 MHz, DMSO-d6) δ [ppm]: 0.835 (0.42), 0.852 (1.06), 0.868 (1.36), 0.887 (1.07), 0.906 (1.98), 0.924 (0.87), 1.233 (2.60), 1.279 (0.65), 1.290 (0.70), 1.353 (0.46), 1.411 (0.41), 1.986 (0.50), 2.006 (0.56), 2.024 (0.51), 2.065 (0.73), 2.078 (0.78), 2.327 (4.22), 2.331 (2.97), 2.337 (1.34), 2.375 (0.70), 2.390 (0.83), 2.413 (0.84), 2.518 (16.00), 2.523 (11.51), 2.669 (4.28), 2.673 (2.94), 2.678 (1.32), 3.445 (2.02), 4.174 (1.01), 4.191 (1.92), 4.208 (0.99), 4.228 (0.50), 4.234 (0.54), 4.242 (0.51), 4.249 (0.46), 5.760 (2.65), 6.459 (3.66), 6.558 (2.65), 6.871 (1.62), 7.391 (2.90), 7.395 (1.81), 7.402 (1.77), 7.406 (2.99), 8.009 (1.47), 8.014 (1.51), 8.089 (3.39), 8.458 (3.55), 8.462 (2.01), 8.469 (1.86), 8.473 (3.35), 8.584 (1.37), 8.588 (1.38).

### Example 775

### (3R)-2′-[6-amino-5-(trifluoromethyl)pyridin-3-yl]-N-[1-(pyridin-3-yl)cyclobutyl]-5′,6′-dihydrospiro[pyrrolidine-3,4′-pyrrolo[1,2-b]pyrazole]-1-carboxamide

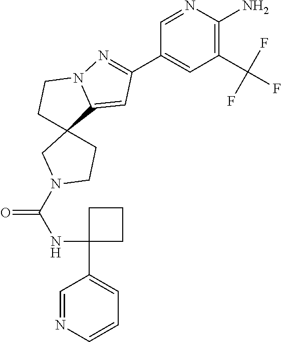 [SMILES: Nc1ncc(-c2cc3n(n2)CC[C@@]32CCN(C(=O)NC3(c4cccnc4)CCC3)C2)cc1C(F)(F)F]

1-(Pyridin-3-yl)cyclobutan-1-amine hydrogen chloride (1/1) (24.6 mg, 133 μmol), CDI (19.8 mg, 122 μmol) and N,N-diisopropylethylamine (120 μL, 670 μmol) were dissolved in DMF (1.5 mL) and stirred for 2 h at rt. 5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (50.0 mg, 80% purity, 111 μmol) was added and the mixture was stirred for 1 h at 60° C. CDI (9.92 mg, 61.1 μmol) and N,N-diisopropylethylamine (58 μL, 330 μmol) were added and the mixture was stirred for 3 h at 60° C. CDI (19.8 mg, 122 μmol) and 1-(pyridin-3-yl)cyclobutan-1-amine hydrogen chloride (1/1) (24.6 mg, 133 μmol) were added again and the mixture was stirred for 2 h at 60° C. The reaction mixture was concentrated, treated with toluene and evaporated again. The residue was purified by preparative HPLC to give 3.50 mg (96% purity, 6% yield) of the title compound.

LC-MS (Method 1): Rₜ=0.96 min; MS (ESIpos): m/z=498 [M+H]⁺

¹H-NMR (400 MHz, CHLOROFORM-d) δ [ppm]: 1.814 (0.43), 1.831 (0.78), 1.837 (0.68), 1.843 (0.58), 1.849 (0.52), 1.854 (0.70), 1.860 (0.96), 1.877 (0.55), 1.882 (0.54), 1.990 (0.40), 2.008 (0.61), 2.022 (1.17), 2.037 (1.18), 2.045 (0.70), 2.052 (0.99), 2.060 (1.00), 2.066 (0.62), 2.075 (0.75), 2.082 (0.68), 2.088 (0.75), 2.097 (0.48), 2.103 (0.43), 2.111 (0.40), 2.143 (0.59), 2.161 (1.17), 2.174 (0.60), 2.180 (0.75), 2.192 (0.83), 2.211 (0.44), 2.431 (0.74), 2.452 (1.26), 2.462 (1.77), 2.470 (1.09), 2.479 (2.67), 2.484 (1.66), 2.495 (1.58), 2.502 (1.30), 2.512 (1.70), 2.529 (0.96), 2.545 (1.20), 2.548 (1.19), 2.554 (1.24), 2.562 (2.37), 2.568 (1.66), 2.576 (2.14), 2.579 (2.46), 2.591 (1.60), 2.595 (2.15), 3.409 (1.30), 3.434 (3.15), 3.444 (1.29), 3.451 (1.03), 3.463 (4.57), 3.469 (2.11), 3.488 (2.29), 3.537 (0.66), 3.550 (0.82), 3.557 (0.99), 3.561 (0.71), 3.570 (0.85), 3.575 (0.71), 3.581 (0.58), 3.595 (0.43), 4.177 (2.92), 4.194 (5.30), 4.212 (2.69), 4.779 (2.82), 4.949 (4.62), 6.102 (8.82), 7.187 (1.60), 7.195 (16.00), 7.205 (1.77), 7.207 (1.71), 7.217 (1.68), 7.219 (1.63), 7.771 (1.34), 7.775 (1.66), 7.781 (1.41), 7.790 (1.27), 7.795 (1.55), 7.796 (1.56), 7.800 (1.25), 8.025 (2.89), 8.029 (2.94), 8.398 (2.36), 8.401 (2.47), 8.410 (2.37), 8.413 (2.29), 8.515 (2.78), 8.519 (2.76), 8.617 (2.99), 8.621 (2.95).

### Example 776

### (3R)-2′-[6-amino-5-(trifluoromethyl) pyridin-3-yl]-N-[(1S)-1-(3-chloropyridin-4-yl)ethyl]-5′,6′-dihydrospiro[pyrrolidine-3,4′-pyrrolo[1,2-b]pyrazole]-1-carboxamide

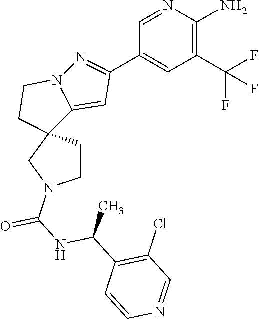 [SMILES: C[C@H](NC(=O)N1CC[C@@]2(CCn3nc(-c4cnc(N)c(C(F)(F)F)c4)cc32)C1)c1ccncc1Cl]

(1S)-1-(3-Chloropyridin-4-yl)ethan-1-amine hydrogen chloride (1/1) (34.5 mg, 179 μmol), CDI (31.6 mg, 195 μmol) and N,N-diisopropylethylamine (110 μL, 650 μmol) were dissolved in DMF (1 mL) and stirred for 1 h at rt. A solution of 5-[(3R)-5′,6′-dihydrospiro[pyrrolidine-3,4′-pyrrolo[1,2-b]pyrazol]-2′-yl]-3-(trifluoromethyl)pyridin-2-amine hydrogen chloride (1/1) (75.0 mg, 78% purity, 163 μmol) in DMF (1 mL) was added and the mixture was stirred over night at rt. The reaction mixture was purified by preparative HPLC to give 33.0 mg (95% purity, 38% yield) of the title compound.

LC-MS (Method 1): Rₜ=0.99 min; MS (ESIpos): m/z=507 [M+H]⁺

¹H-NMR (400 MHz, DMSO-d6) δ [ppm]: 0.934 (1.63), 0.951 (1.63), 1.336 (9.87), 1.354 (9.96), 2.067 (1.40), 2.085 (2.44), 2.518 (3.49), 2.523 (2.80), 2.565 (2.64), 3.437 (1.55), 3.462 (3.32), 3.498 (2.89), 3.522 (1.98), 3.539 (1.57), 3.555 (0.97), 3.565 (0.77), 4.175 (2.75), 4.193 (4.49), 4.210 (2.63), 5.052 (1.65), 5.069 (2.52), 5.088 (1.60), 6.470 (16.00), 6.553 (7.14), 6.778 (2.38), 6.796 (2.28), 7.454 (3.57), 7.467 (3.56), 8.014 (4.21), 8.019 (4.20), 8.436 (4.22), 8.448 (4.00), 8.525 (11.64), 8.589 (3.92), 8.593 (3.77).

### Example 777

### 2′-(6-amino-5-cyanopyridin-3-yl)-N-[1-(pyridin-4-yl)cyclobutyl]-5′,6′-dihydrospiro[pyrrolidine-3,4′-pyrrolo[1,2-b]pyrazole]-1-carboxamide (Enantiomer 2)

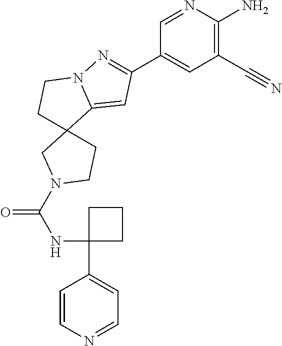 [SMILES: N#Cc1cc(-c2cc3n(n2)CCC32CCN(C(=O)NC3(c4ccncc4)CCC3)C2)cnc1N]

1-(Pyridin-4-yl)cyclobutan-1-amine (75.8 mg, 511 μmol), CDI (76.0 mg, 469 μmol) and N,N-diisopropylethylamine (370 μL, 2.1 mmol were dissolved in DMF (6.4 mL) and stirred for 2 h at rt. (Rac)-2-Amino-5-[5′,6′-dihydrospiro[pyrrolidine-3,4′-pyrrolo[1,2-b]pyrazol]-2′-yl]pyridine-3-carbonitrile hydrochloride (150 mg, 90% purity, 426 μmol) was added and the mixture was stirred for 2 h at 60° C. The reaction mixture was allowed to cool down and concentrated. The residue was treated with toluene and evaporated again. The crude product was purified by column chromatography (silica gel, dichloromethane/methanol (0-10%) gradient) to give 161 mg of the title compound as a racemic mixture.

The racemic mixture was separated into enantiomers by preparative chiral HPLC to give 17.5 mg (95% purity, 9% yield) of the title compound (enantiomer 2 Rₜ=16.1-19.3 min).

Preparative Chiral HPLC Method:

Instrument: PrepCon Labomatic HPLC-3; Column: YMC Cellulose SB 10μ, 250×50; eluent A: methyl tert-butyl ether+0.1 vol % diethylamine; eluent B: acetonitrile+0.1 vol % diethylamine; isocratic: 90% B; flow: 100 mL/min; temperature: 25° C.; UV: 254 nm

Analytical Chiral HPLC Method:

Instrument: Thermo Fisher UltiMate 3000; Column: YMC Cellulose SB 3μ, 100×4.6; eluent A: methyl tert-butyl ether+0.1 vol % diethylamine; eluent B: acetonitrile; isocratic: 90% B; flow: 1.4 mL/min; temperature: 25° C.; UV: 254 nm

Analytical chiral HPLC: Rₜ=4.29 min

[α]²⁰_{D}: +64.0° (c=1.00, CHCl₃)

LC-MS (Method 1): Rₜ=0.83 min; MS (ESIpos): m/z=455 [M+H]⁺

¹H NMR (CHLOROFORM-d, 400 MHz): δ (ppm) 8.62 (d, 1H), 8.58 (br d, 2H), 8.07 (d, 1H), 7.35-7.39 (m, 2H), 6.15 (s, 1H), 5.22 (s, 2H), 4.83 (s, 1H), 4.27 (t, 2H), 3.61-3.69 (m, 1H), 3.49-3.59 (m, 3H), 2.54-2.70 (m, 4H), 2.44-2.53 (m, 2H), 2.27 (dt, 1H), 2.07-2.20 (m, 2H), 1.88-2.00 (m, 1H).

### Example 778

### (3R)-2′-[6-amino-5-(trifluoromethyl)pyridin-3-yl]-N-[1-(3-methylpyridin-4-yl)ethyl]-5′,6′-dihydro-1H-spiro[pyrrolidine-3,4′-pyrrolo[1,2-b]pyrazole]-1-carboxamide (Diastereomer 1 and Diastereomer 2)

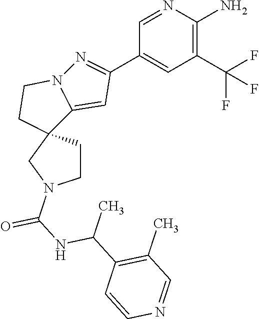 [SMILES: Cc1cnccc1C(C)NC(=O)N1CC[C@@]2(CCn3nc(-c4cnc(N)c(C(F)(F)F)c4)cc32)C1]

1-(3-Methylpyridin-4-yl)ethan-1-amine dihydrogen chloride (1/2) (63.9 mg, 306 μmol), CDI (54.1 mg, 334 μmol) and N,N-diisopropylethylamine (240 μL, 1.4 mmol) were dissolved in DMF (3 mL) and stirred for 1 h at rt. 5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (125 mg, 80% purity, 278 μmol) was added and stirred for 3 h at 60° C. The reaction mixture was allowed to cool down and purified by preparative HPLC to give 45.0 mg (99% purity, 33% yield) of the title compound.

LC-MS (Method 1): Rₜ=0.91 min; MS (ESIpos): m/z=487 [M+H]⁺

¹H-NMR (400 MHz, DMSO-d6) δ [ppm]: 1.295 (4.27), 1.300 (4.43), 1.312 (4.40), 1.318 (4.29), 2.049 (1.00), 2.066 (1.70), 2.075 (4.17), 2.306 (16.00), 2.318 (0.60), 2.323 (1.04), 2.327 (1.38), 2.332 (0.93), 2.337 (0.41), 2.518 (5.83), 2.523 (4.42), 2.660 (0.41), 2.665 (0.96), 2.669 (1.32), 2.674 (0.92), 2.679 (0.40), 3.418 (1.20), 3.444 (3.50), 3.456 (2.87), 3.481 (0.82), 3.518 (0.58), 3.545 (0.45), 3.565 (0.51), 4.169 (1.53), 4.187 (2.87), 4.200 (1.43), 4.913 (1.04), 4.931 (1.64), 4.949 (1.05), 6.444 (2.52), 6.451 (6.30), 6.549 (4.55), 6.618 (1.02), 6.632 (1.25), 6.636 (1.22), 0.650 (0.94), 7.324 (1.35), 7.336 (1.37), 7.378 (1.39), 7.391 (1.41), 8.007 (2.97), 8.012 (2.99), 8.271 (3.11), 8.273 (2.70), 8.277 (3.15), 8.304 (1.43), 8.317 (1.36), 8.341 (1.65), 8.354 (1.55), 8.578 (2.60), 8.580 (2.61), 8.583 (2.57).

### Example 779

### (3R)-2′-[6-amino-5-(trifluoromethyl)pyridin-3-yl]-N-[cyclopropyl(phenyl)methyl]-5′,6′-dihydrospiro[pyrrolidine-3,4′-pyrrolo[1,2-b]pyrazole]-1-carboxamide (Diastereomer 1)

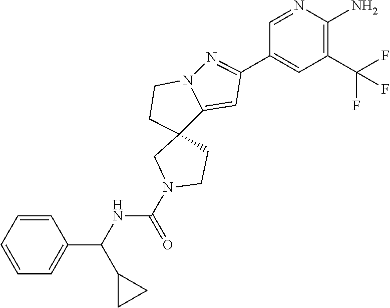 [SMILES: Nc1ncc(-c2cc3n(n2)CC[C@@]32CCN(C(=O)NC(c3ccccc3)C3CC3)C2)cc1C(F)(F)F]

1-Cyclopropyl-1-phenylmethanamine (98.2 mg, 667 μmol), CDI (108 mg, 667 μmol) and N,N-diisopropylethylamine (387 μL, 2.2 mmol) were dissolved in DMF (10 mL) and stirred for 1 h at room temperature. 5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (200 mg, 556 μmol) was added and the mixture was stirred for 2 h at room temperature. The reaction mixture was concentrated and diluted with water. The aqueous phase was extracted with dichloromethane. The combined organic layers were concentrated. The residue was purified by column chromatography (silica gel, ethyl acetate/methanol (0-10%) gradient) to give 222 mg (80% yield) of the title compound as a mixture of diastereomers.

The mixture was separated into diastereomers by preparative chiral HPLC to give 110 mg (95% purity, 34% yield) of the title compound (diastereomer 1 Rₜ=5.2-6.2 min).

Preparative Chiral HPLC Method:

Instrument: PrepCon Labomatic HPLC-2; Column: YMC Cellulose SB 5μ, 250×50; eluent A: methyl tert-butyl ether+0.1 vol % diethylamine; eluent B: acetonitrile+0.1 vol % diethylamine; isocratic: 20% B; flow: 50 mL/min; temperature: 25° C.; UV: 254 nm

Analytical Chiral HPLC Method:

Instrument: Thermo Fisher UltiMate 3000; Column: YMC Cellulose SB 3μ, 100×4.6; eluent A: methyl tert-butyl ether+0.1 vol % diethylamine; eluent B: acetonitrile; isocratic: 20% B; flow: 1.4 mL/min; temperature: 25° C.; UV: 254 nm

Analytical chiral HPLC: Rₜ=2.26 min

[α]²⁰_{D}: +86.6° (c=1.00, DMSO)

LC-MS (Method 1): Rₜ=1.12 min; MS (ESIpos): m/z=497 [M+H]⁺

¹H NMR (DMSO-d₆, 400 MHz): δ (ppm) 8.57 (d, 1H), 8.00 (d, 1H), 7.39-7.44 (m, 2H), 7.27-7.33 (m, 2H), 7.17-7.23 (m, 1H), 6.65 (d, 1H), 6.55 (s, 2H), 6.44 (s, 1H), 4.19 (t, 2H), 4.06 (t, 1H), 3.54-3.62 (m, 1H), 3.40-3.50 (m, 3H), 2.52-2.56 (m, 2H), 2.01-2.13 (m, 2H), 1.14-1.27 (m, 1H), 0.42-0.52 (m, 2H), 0.27-0.39 (m, 2H).

### Example 780

### (3R)-2′-[6-amino-5-(trifluoromethyl)pyridin-3-yl]-N-[cyclopropyl(phenyl)methyl]-5′,6′-dihydrospiro[pyrrolidine-3,4′-pyrrolo[1,2-b]pyrazole]-1-carboxamide (Diastereomer 2)

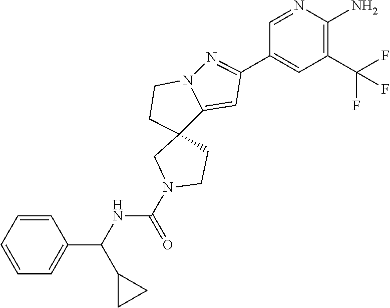 [SMILES: Nc1ncc(-c2cc3n(n2)CC[C@@]32CCN(C(=O)NC(c3ccccc3)C3CC3)C2)cc1C(F)(F)F]

For the preparation of the diastereomeric mixture of the title compound see Example 779. The diastereomers were separated by preparative chiral HPLC (method see Example 779) to give 115 mg (95% purity, 35% yield) of the title compound (diastereomer 2 Rₜ=7.9-9.9 min).

Analytical chiral HPLC (method see Example 779): Rₜ=3.81 min.

[α]²⁰_{D}: +28.5° (c=1.00, DMSO)

LC-MS (Method 1): Rₜ=1.12 min; MS (ESIpos): m/z=497 [M+H]⁺

¹H NMR (DMSO-d₆, 400 MHz): δ (ppm) 8.58 (d, 1H), 8.00 (d, 1H), 7.36-7.41 (m, 2H), 7.25-7.30 (m, 2H), 7.16-7.21 (m, 1H), 6.65 (d, 1H), 6.55 (s, 2H), 6.43 (s, 1H), 4.19 (t, 2H), 4.06 (t, 1H), 3.52-3.59 (m, 1H), 3.41-3.51 (m, 3H), 2.52-2.57 (m, 2H), 2.01-2.12 (m, 2H), 1.16-1.26 (m, 1H), 0.42-0.54 (m, 2H), 0.28-0.40 (m, 2H).

### Example 781

### (3R)-2′-[6-amino-5-(trifluoromethyl)pyridin-3-yl]-N-[1-(pyridin-2-yl)cyclobutyl]-5′,6′-dihydrospiro[pyrrolidine-3,4′-pyrrolo[1,2-b]pyrazole]-1-carboxamide

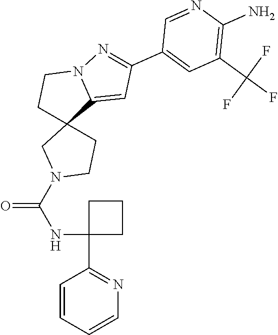 [SMILES: Nc1ncc(-c2cc3n(n2)CC[C@@]32CCN(C(=O)NC3(c4ccccn4)CCC3)C2)cc1C(F)(F)F]

1-(Pyridin-2-yl)cyclobutan-1-amine (19.8 mg, 133 μmol), CDI (19.8 mg, 122 μmol) and N,N-diisopropylethylamine (120 μL, 670 μmol) were dissolved in DMF (1.5 mL) and stirred for 1 h at rt. A solution of 5-[(3R)-5′,6′-dihydrospiro[pyrrolidine-3,4′-pyrrolo[1,2-b]pyrazol]-2′-yl]-3-(trifluoromethyl)pyridin-2-amine hydrogen chloride (1/1) (50.0 mg, 80% purity, 111 μmol) in DMF was added and the mixture was stirred over night at 60° C. The reaction mixture was allowed to cool down, concentrated, treated with toluene and evaporated again. The residue was purified by preparative HPLC followed by preparative TLC (dichloromethane/methanol 9:1 gradient) to give 12.4 mg (100% purity, 22% yield) of the title compound.

LC-MS (Method 1): Rₜ=1.02 min; MS (ESIpos): m/z=498 [M+H]⁺

¹H-NMR (400 MHz, DMSO-d6) δ [ppm]: 0.852 (1.07), 0.868 (1.92), 0.886 (1.59), 0.906 (2.94), 0.924 (1.34), 1.232 (2.87), 1.279 (0.80), 1.297 (0.97), 1.353 (0.64), 1.392 (0.57), 1.412 (0.60), 1.431 (0.44), 1.862 (0.48), 1.878 (0.89), 1.888 (0.80), 1.905 (1.30), 1.921 (0.77), 1.927 (0.67), 1.944 (0.42), 1.962 (0.69), 1.978 (1.23), 2.001 (1.01), 2.017 (0.62), 2.069 (1.25), 2.084 (2.20), 2.099 (2.55), 2.116 (1.09), 2.129 (0.54), 2.327 (4.09), 2.331 (2.93), 2.337 (1.44), 2.373 (1.89), 2.394 (2.00), 2.409 (1.21), 2.518 (16.00), 2.523 (11.09), 2.561 (5.22), 2.577 (4.59), 2.589 (2.06), 2.594 (2.03), 2.617 (0.81), 2.669 (4.12), 2.673 (2.82), 2.678 (1.25), 3.483 (6.89), 3.569 (0.65), 3.587 (1.18), 3.627 (0.46), 4.184 (3.08), 4.202 (5.29), 4.219 (3.01), 4.228 (0.99), 4.234 (0.87), 4.242 (0.81), 4.248 (0.76), 5.760 (15.19), 6.476 (12.13), 6.558 (7.76), 6.792 (5.11), 7.158 (2.22), 7.161 (2.49), 7.170 (2.24), 7.173 (2.65), 7.177 (2.62), 7.179 (2.43), 7.189 (2.53), 7.191 (2.47), 7.360 (3.66), 7.379 (4.00), 7.645 (2.30), 7.649 (2.49), 7.663 (2.92), 7.668 (2.84), 7.683 (1.76), 7.688 (1.85), 8.011 (4.43), 8.016 (4.44), 8.089 (5.24), 8.519 (2.64), 8.521 (2.76), 8.523 (3.05), 8.526 (2.58), 8.531 (2.65), 8.533 (2.85), 8.535 (2.76), 8.538 (2.22), 8.590 (4.13), 8.594 (4.02).

### Example 782

### (3R)-2′-[6-amino-5-(trifluoromethyl)pyridin-3-yl]-N-[2-methyl-1-(pyridin-4-yl)propyl]-5′,6′-dihydro-1H-spiro[pyrrolidine-3,4′-pyrrolo[1,2-b]pyrazole]-1-carboxamide (Diastereomer 1 and Diastereomer 2)

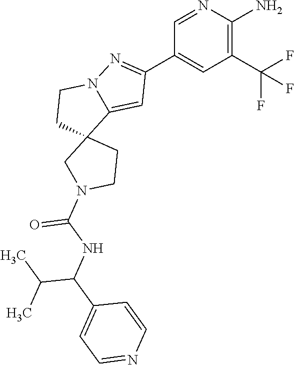 [SMILES: CC(C)C(NC(=O)N1CC[C@@]2(CCn3nc(-c4cnc(N)c(C(F)(F)F)c4)cc32)C1)c1ccncc1]

2-Methyl-1-(pyridin-4-yl)propan-1-amine (37.6 mg, 250 μmol) was dissolved in DMF (5 mL). CDI (40.6 mg, 250 μmol) and N,N-diisopropylethylamine (120 μL, 830 μmol) were added and stirred for 1 h at rt. 5-[(3R)-5′,6′-Ddihydrospiro[pyrrolidine-3,4′-pyrrolo[1,2-b]pyrazol]-2′-yl]-3-(trifluoromethyl)pyridin-2-amine hydrogen chloride (1/1) (75.0 mg, 208 μmol) was added and the mixture was stirred over night at rt. The reaction mixture was diluted with water and extracted three times with dichloromethane. The organic phase was dried over a hydrophobic filter and concentrated. The residue was purified by preparative HPLC to give 50.0 mg (100% purity, 48% yield) of the title compound.

LC-MS (Method 1): Rₜ=0.94 min; MS (ESIpos): m/z=500 [M+H]⁺

¹H-NMR (400 MHz, DMSO-d6) δ [ppm]: 0.663 (0.97), 0.679 (10.34), 0.684 (10.23), 0.696 (10.38), 0.701 (10.08), 0.733 (0.76), 0.750 (0.69), 0.936 (9.00), 0.953 (9.40), 0.969 (8.96), 0.986 (9.18), 1.978 (0.82), 1.986 (0.97), 1.994 (1.32), 2.003 (1.45), 2.010 (1.67), 2.016 (1.56), 2.026 (1.85), 2.041 (2.68), 2.055 (3.57), 2.067 (4.25), 2.085 (1.92), 2.097 (0.80), 2.332 (2.74), 2.336 (1.16), 2.518 (16.00), 2.523 (13.66), 2.673 (2.78), 2.678 (1.21), 3.382 (0.50), 3.405 (2.42), 3.415 (2.05), 3.430 (3.66), 3.440 (3.35), 3.450 (1.25), 3.459 (1.08), 3.477 (4.92), 3.491 (4.16), 3.502 (2.46), 3.511 (1.73), 3.516 (2.56), 3.566 (0.63), 3.585 (1.16), 3.592 (0.76), 3.599 (0.84), 3.608 (0.87), 3.625 (0.47), 4.166 (4.67), 4.183 (8.82), 4.200 (4.54), 4.334 (1.39), 4.350 (1.76), 4.356 (2.88), 4.372 (2.92), 4.378 (1.73), 4.394 (1.36), 4.485 (0.40), 6.425 (15.08), 6.430 (7.39), 6.447 (3.27), 6.471 (1.96), 6.548 (11.81), 7.315 (6.36), 7.319 (3.76), 7.326 (3.92), 7.330 (6.78), 7.338 (6.62), 7.341 (3.92), 7.349 (3.89), 7.353 (6.53), 7.986 (3.64), 7.991 (3.98), 7.997 (3.79), 8.002 (3.57), 8.451 (7.85), 8.454 (4.30), 8.461 (4.34), 8.466 (8.69), 8.468 (9.72), 8.471 (4.64), 8.478 (4.20), 8.482 (7.40), 8.563 (6.37), 8.569 (6.18).

### Example 783

### (3R)-2′-[6-amino-5-(trifluoromethyl)pyridin-3-yl]-N-[2-methyl-1-(pyridin-4-yl)propyl]-5′,6′-dihydro-1H-spiro[pyrrolidine-3,4′-pyrrolo[1,2-b]pyrazole]-1-carboxamide (Diastereomer 1)

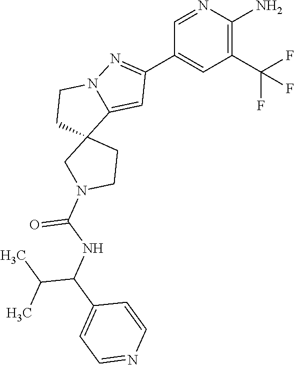 [SMILES: CC(C)C(NC(=O)N1CC[C@@]2(CCn3nc(-c4cnc(N)c(C(F)(F)F)c4)cc32)C1)c1ccncc1]

The title compound from Example 782 was separated into diastereomers by preparative chiral HPLC to give 20.8 mg (97% purity, 19% yield) of the title compound (diastereomer 1 Rₜ=10.84-12.96 min).

Preparative Chiral HPLC Method:

Instrument: PrepCon Labomatic HPLC-3; Column: YMC Cellulose SC 5p, 250×50; eluent A: hexane+0.1 vol % diethylamine; eluent B: ethanol; isocratic: 50% B; flow: 60 mL/min; temperature: 25° C.; UV: 280 nm

Analytical Chiral HPLC Method:

Instrument: Thermo Fisher UltiMate 3000; Column: YMC Cellulose SC 3μ, 100×4.6; eluent A: hexane+0.1 vol % diethylamine; eluent B: ethanol; isocratic: 50% B; flow: 1.4 mL/min; temperature: 25° C.; UV: 280 nm

Analytical chiral HPLC: Rₜ=2.11 min

[α]²⁰_{D}: +91.0° (c=1.00, methanol)

LC-MS (Method 1): Rₜ=0.96 min; MS (ESIpos): m/z=500 [M+H]⁺

¹H-NMR (400 MHz, DMSO-d6) δ [ppm]: 0.679 (14.84), 0.696 (15.36), 0.795 (0.77), 0.798 (0.60), 0.814 (0.71), 0.822 (0.63), 0.836 (0.51), 0.851 (0.45), 0.862 (0.49), 0.905 (0.63), 0.936 (15.52), 0.953 (16.00), 1.006 (0.58), 1.035 (0.45), 1.084 (1.24), 1.204 (0.44), 1.232 (1.12), 1.259 (1.72), 1.352 (2.20), 1.961 (0.53), 1.978 (1.26), 1.994 (1.79), 1.999 (1.55), 2.011 (1.64), 2.016 (1.96), 2.033 (1.86), 2.040 (2.17), 2.055 (3.25), 2.067 (4.04), 2.085 (1.89), 2.097 (0.76), 2.114 (0.43), 2.327 (3.07), 2.332 (2.20), 2.336 (0.99), 2.518 (14.31), 2.523 (12.39), 2.562 (0.60), 2.673 (2.23), 2.678 (0.97), 3.382 (0.91), 3.401 (2.05), 3.415 (3.37), 3.426 (2.70), 3.440 (5.45), 3.477 (5.73), 3.502 (2.95), 3.566 (1.07), 3.584 (2.11), 3.592 (1.42), 3.599 (1.59), 3.610 (1.52), 3.625 (0.83), 4.166 (4.46), 4.184 (8.56), 4.201 (4.27), 4.350 (2.46), 4.372 (4.80), 4.394 (2.39), 6.430 (11.96), 6.447 (3.93), 6.549 (11.21), 7.338 (10.80), 7.341 (6.74), 7.349 (6.90), 7.353 (11.04), 7.997 (6.27), 8.002 (6.31), 8.468 (12.78), 8.471 (7.23), 8.478 (6.80), 8.483 (11.52), 8.565 (5.80), 8.569 (5.71), 10.851 (0.62).

### Example 784

### (3R)-2′-[6-amino-5-(trifluoromethyl)pyridin-3-yl]-N-[2-methyl-1-(pyridin-4-yl)propyl]-5′,6′-dihydro-1H-spiro[pyrrolidine-3,4′-pyrrolo[1,2-b]pyrazole]-1-carboxamide (Diastereomer 2)

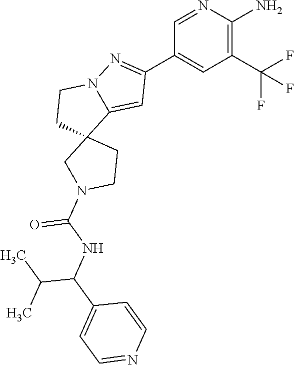 [SMILES: CC(C)C(NC(=O)N1CC[C@@]2(CCn3nc(-c4cnc(N)c(C(F)(F)F)c4)cc32)C1)c1ccncc1]

For the preparation of the diastereomeric mixture of the title compound see Example 782. The diastereomers were separated by preparative chiral HPLC (method see Example 783) to give 12.6 mg (100% purity, 12% yield) of the title compound (diastereomer 2 Rₜ=19.29-22.77 min).

Analytical chiral HPLC (method see Example 783): Rₜ=3.79 min.

[α]²⁰_{D}: +65.9° (c=1.00, methanol)

LC-MS (Method 1): Rₜ=0.96 min; MS (ESIpos): m/z=500 [M+H]⁺

¹H NMR (DMSO-d₆, 400 MHz): δ (ppm) 8.56 (d, 1H), 8.44-8.47 (m, 2H), 7.99 (d, 1H), 7.30-7.34 (m, 2H), 6.53 (s, 2H), 6.48 (d, 1H), 6.42 (s, 1H), 4.35 (t, 1H), 4.18 (t, 2H), 3.44-3.57 (m, 6H), 1.96-2.12 (m, 3H), 0.97 (d, 3H), 0.69 (d, 3H).

### Example 785

### (3R)-2′-[6-amino-5-(trifluoromethyl)pyridin-3-yl]-N-[1-(3-fluoropyridin-4-yl)ethyl]-5′,6′-dihydro-1H-spiro[pyrrolidine-3,4′-pyrrolo[1,2-b]pyrazole]-1-carboxamide (Diastereomer 1 and Diastereomer 2)

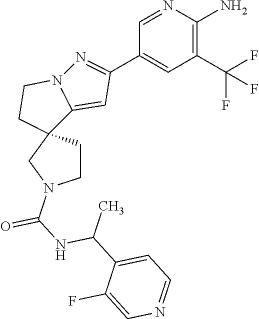 [SMILES: CC(NC(=O)N1CC[C@@]2(CCn3nc(-c4cnc(N)c(C(F)(F)F)c4)cc32)C1)c1ccncc1F]

1-(3-Fluoropyridin-4-yl)ethan-1-amine (76.9 mg, 548 μmol), CDI (97.0 mg, 598 μmol) and N,N-diisopropylethylamine (350 μL, 2.0 mmol) were dissolved in DMF (5 mL) and stirred for 1 h at rt. A solution of 5-[(3R)-5′,6′-dihydrospiro[pyrrolidine-3,4′-pyrrolo[1,2-b]pyrazol]-2′-yl]-3-(trifluoromethyl)pyridin-2-amine hydrogen chloride (1/1) (230 mg, 78% purity, 499 μmol) in DMF (1 mL) was added and the mixture was stirred over night at rt. The reaction mixture was purified by preparative HPLC to give 94.0 mg (95% purity, 37% yield) of the title compound.

LC-MS (Method 1): Rₜ=0.94 min; MS (ESIpos): m/z=491 [M+H]⁺

¹H-NMR (400 MHz, DMSO-d6) δ [ppm]: 0.932 (1.01), 0.948 (1.00), 1.361 (7.89), 1.366 (8.12), 1.378 (8.09), 1.383 (7.90), 2.075 (4.08), 2.083 (3.23), 2.337 (1.31), 2.518 (16.00), 2.523 (12.23), 2.565 (3.29), 2.679 (1.23), 3.438 (2.37), 3.464 (5.76), 3.483 (4.25), 3.509 (1.50), 3.542 (1.10), 3.567 (0.64), 3.592 (0.84), 4.177 (3.01), 4.193 (5.36), 4.209 (2.79), 5.052 (1.23), 5.057 (1.34), 5.070 (1.93), 5.076 (1.94), 5.088 (1.31), 5.093 (1.25), 6.474 (14.36), 6.550 (9.40), 6.686 (1.88), 6.698 (2.06), 6.704 (2.09), 6.717 (1.67), 7.414 (1.27), 7.426 (1.53), 7.443 (1.26), 7.484 (1.39), 7.497 (1.60), 7.513 (1.38), 8.014 (5.71), 8.019 (5.67), 8.346 (2.12), 8.359 (2.05), 8.381 (2.56), 8.384 (2.47), 8.396 (2.38), 8.451 (4.61), 8.456 (8.49), 8.461 (4.31), 8.588 (5.29), 8.593 (5.07).

### Example 786

### (3R)-2′-[6-amino-5-(trifluoromethyl)pyridin-3-yl]-N-[1-(3-fluoropyridin-4-yl)ethyl]-5′,6′-dihydro-1H-spiro[pyrrolidine-3,4′-pyrrolo[1,2-b]pyrazole]-1-carboxamide (Diastereomer 1)

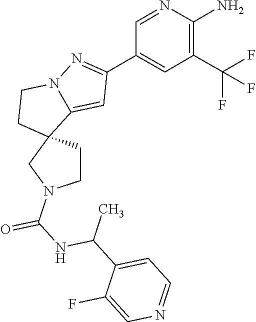 [SMILES: CC(NC(=O)N1CC[C@@]2(CCn3nc(-c4cnc(N)c(C(F)(F)F)c4)cc32)C1)c1ccncc1F]

The title compound from Example 785 was separated into diastereomers by preparative chiral HPLC (diastereomer 1 Rₜ=8.8-12.0 min).

Preparative Chiral HPLC Method:

Instrument: Sepiatec: Prep SFC100; Column: Chiral Art Cellulose-SC 5μ, 250×50; eluent A: CO2; eluent B: 2-propanol+0.4 vol % diethylamine; isocratic: 35% B; flow: 100 mL/min; temperature: 40° C.; BPR: 150 bar; UV: 267 nm

Analytical Chiral HPLC Method:

Instrument: Waters Acquity UPC2 QDA; Column: Chiral Art Cellulose-SB 3μ, 100×4.6; eluent A: CO2; eluent B: 2-propanol+0.4 vol % diethylamine; isocratic: 35% B; flow: 4 mL/min; temperature: 37.5° C.; BPR: 100 bar; UV: 254 nm

Analytical chiral HPLC: Rₜ=3.04 min

The crude fraction was purified by preparative HPLC to give 32.0 mg (99% purity, 35% yield) of the title compound.

[α]²⁰_{D}: +20.1° (c=1.00, DMSO)

LC-MS (Method 1): Rₜ=0.94 min; MS (ESIpos): m/z=490 [M+H]⁺

¹H-NMR (400 MHz, DMSO-d6) δ [ppm]: 1.366 (9.23), 1.384 (9.31), 2.064 (1.34), 2.077 (2.07), 2.083 (2.42), 2.100 (1.16), 2.518 (1.87), 2.523 (1.77), 2.565 (2.66), 3.438 (1.51), 3.464 (4.01), 3.488 (3.26), 3.513 (1.47), 3.525 (0.91), 3.542 (1.50), 3.551 (0.78), 3.558 (0.95), 3.568 (0.80), 4.173 (2.76), 4.192 (4.43), 4.209 (2.66), 5.053 (1.48), 5.071 (2.26), 5.090 (1.45), 6.474 (16.00), 6.550 (7.06), 6.699 (2.32), 6.718 (2.22), 7.414 (1.75), 7.427 (2.11), 7.431 (2.08), 7.444 (1.73), 8.015 (4.11), 8.020 (4.12), 8.346 (2.92), 8.358 (2.83), 8.451 (5.97), 8.456 (5.88), 8.590 (3.84), 8.594 (3.77).

### Example 787

### (3R)-2′-[6-amino-5-(trifluoromethyl)pyridin-3-yl]-N-[1-(3-fluoropyridin-4-yl)ethyl]-5′,6′-dihydro-1H-spiro[pyrrolidine-3,4′-pyrrolo[1,2-b]pyrazole]-1-carboxamide (Diastereomer 2)

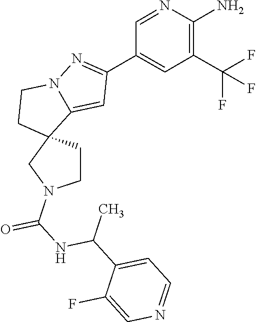 [SMILES: CC(NC(=O)N1CC[C@@]2(CCn3nc(-c4cnc(N)c(C(F)(F)F)c4)cc32)C1)c1ccncc1F]

For the preparation of the diastereomeric mixture of the title compound see Example 785. The diastereomers were separated by preparative chiral HPLC (method see Example 786) (diastereomer 2 Rₜ=16.5-22.0 min).

Analytical chiral HPLC (method see Example 786): Rₜ=5.75 min

The crude fraction was purified by preparative HPLC to give 36.0 mg (99% purity, 40% yield) of the title compound.

[α]²⁰_{D}: +91.4° (c=1.00, DMSO)

LC-MS (Method 1): Rₜ=0.94 min; MS (ESIpos): m/z=490 [M+H]⁺

¹H-NMR (400 MHz, DMSO-d6) δ [ppm]: 1.361 (15.91), 1.379 (16.00), 2.081 (3.36), 2.088 (3.84), 2.106 (1.83), 2.119 (0.62), 2.332 (0.54), 2.518 (3.21), 2.523 (2.64), 2.567 (4.59), 3.422 (0.91), 3.440 (3.32), 3.466 (7.65), 3.483 (6.90), 3.509 (1.88), 3.577 (0.99), 3.594 (1.94), 3.602 (1.32), 3.610 (1.41), 3.619 (1.46), 3.635 (0.71), 4.177 (4.93), 4.195 (8.34), 4.213 (4.74), 5.042 (0.59), 5.059 (2.59), 5.077 (3.97), 5.095 (2.56), 5.113 (0.57), 6.473 (11.38), 6.552 (12.20), 6.687 (4.11), 6.706 (3.95), 7.485 (3.25), 7.497 (3.82), 7.501 (3.81), 7.514 (3.26), 8.016 (6.78), 8.021 (6.85), 8.381 (6.04), 8.384 (5.92), 8.394 (5.73), 8.396 (5.67), 8.456 (10.28), 8.461 (10.20), 8.589 (6.31), 8.593 (6.27).

### Example 788

### (3R)-2′-[6-amino-5-(trifluoromethyl)pyridin-3-yl]-N-[(1R)-1-(3-chloropyridin-4-yl)ethyl]-5′,6′-dihydrospiro[pyrrolidine-3,4′-pyrrolo[1,2-b]pyrazole]-1-carboxamide

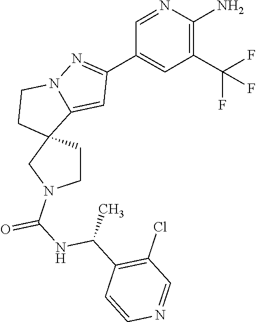 [SMILES: C[C@@H](NC(=O)N1CC[C@@]2(CCn3nc(-c4cnc(N)c(C(F)(F)F)c4)cc32)C1)c1ccncc1Cl]

(1R)-1-(3-Chloropyridin-4-yl)ethan-1-amine (28.0 mg, 179 μmol), CDI (31.6 mg, 195 μmol) and N,N-diisopropylethylamine (110 μL, 650 μmol) were dissolved in DMF (1 mL) and stirred for 1 h at rt. A solution of 5-[(3R)-5′,6′-dihydrospiro[pyrrolidine-3,4′-pyrrolo[1,2-b]pyrazol]-2′-yl]-3-(trifluoromethyl)pyridin-2-amine hydrogen chloride (1/1) (75.0 mg, 78% purity, 163 μmol) in DMF (1 mL) was added and the mixture was stirred over night at rt. The reaction mixture was purified by preparative HPLC to give 24.0 mg (99% purity, 29% yield) of the title compound.

LC-MS (Method 1): Rₜ=0.99 min; MS (ESIpos): m/z=507 [M+H]⁺

¹H-NMR (400 MHz, DMSO-d6) δ [ppm]: 1.332 (14.52), 1.349 (14.56), 2.085 (3.01), 2.092 (3.23), 2.327 (3.34), 2.331 (2.33), 2.337 (1.03), 2.518 (13.69), 2.523 (9.87), 2.568 (3.53), 2.673 (2.31), 2.678 (1.04), 3.420 (0.76), 3.440 (2.84), 3.465 (5.43), 3.490 (4.05), 3.516 (1.61), 3.585 (0.77), 3.603 (1.48), 3.628 (1.16), 4.179 (4.16), 4.197 (7.28), 4.214 (3.99), 5.037 (0.53), 5.055 (2.39), 5.073 (3.73), 5.090 (2.36), 5.108 (0.50), 6.472 (9.38), 6.554 (10.26), 6.762 (3.60), 6.780 (3.44), 7.531 (5.66), 7.543 (5.80), 8.015 (5.71), 8.020 (5.76), 8.478 (9.40), 8.490 (8.96), 8.529 (16.00), 8.588 (5.35), 8.592 (5.20).

### Example 789

### (3R)-2′-[6-amino-5-(trifluoromethyl)pyridin-3-yl]-N-[1-(2-methylpyridin-4-yl)ethyl]-5′,6′-dihydro-1H-spiro[pyrrolidine-3,4′-pyrrolo[1,2-b]pyrazole]-1-carboxamide (Diastereomer 1 and Diastereomer 2)

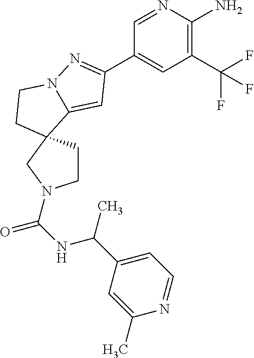 [SMILES: Cc1cc(C(C)NC(=O)N2CC[C@@]3(CCn4nc(-c5cnc(N)c(C(F)(F)F)c5)cc43)C2)ccn1]

1-(2-Methylpyridin-4-yl)ethan-1-amine (24.4 mg, 179 μmol), CDI (31.6 mg, 195 μmol) and N,N-diisopropylethylamine (110 μL, 650 μmol) were dissolved in DMF (1 mL) and stirred for 1 h at rt. A solution of 5-[(3R)-5′,6′-dihydrospiro[pyrrolidine-3,4′-pyrrolo[1,2-b]pyrazol]-2′-yl]-3-(trifluoromethyl)pyridin-2-amine hydrogen chloride (1/1) (75.0 mg, 78% purity, 163 μmol) in DMF (1 mL) was added and the mixture was stirred over night at rt. The reaction mixture was purified by preparative HPLC to give 40.0 mg (95% purity, 48% yield) of the title compound.

LC-MS (Method 1): Rₜ=0.93 min; MS (ESIpos): m/z=487 [M+H]⁺

¹H-NMR (400 MHz, DMSO-d6) δ [ppm]: 1.204 (0.53), 1.221 (0.50), 1.335 (6.35), 1.340 (6.38), 1.353 (6.63), 1.358 (6.27), 1.385 (0.82), 1.401 (0.77), 2.059 (1.44), 2.076 (2.66), 2.084 (2.11), 2.281 (2.26), 2.318 (0.96), 2.407 (14.08), 2.427 (1.12), 2.435 (16.00), 2.456 (2.10), 2.463 (0.80), 2.468 (0.96), 2.518 (13.53), 2.522 (9.75), 2.564 (3.04), 2.604 (0.76), 2.660 (1.01), 3.344 (1.40), 3.361 (0.59), 3.436 (1.64), 3.444 (0.87), 3.461 (4.62), 3.469 (3.46), 3.480 (3.06), 3.495 (1.00), 3.506 (0.84), 3.522 (0.46), 3.539 (0.92), 3.556 (0.62), 3.564 (0.83), 3.580 (0.95), 3.596 (0.54), 3.604 (0.54), 4.175 (2.68), 4.192 (4.88), 4.209 (2.58), 4.755 (1.05), 4.773 (1.55), 4.789 (1.04), 6.469 (5.16), 6.472 (9.51), 6.547 (8.50), 6.566 (1.76), 7.106 (1.34), 7.119 (1.38), 7.141 (1.45), 7.154 (3.91), 7.190 (2.68), 8.013 (4.04), 8.018 (4.12), 8.306 (2.30), 8.319 (2.25), 8.330 (2.50), 8.343 (2.37), 8.588 (4.10).

### Example 790

### (3R)-2′-[6-amino-5-(trifluoromethyl)pyridin-3-yl]-N-[1-(3-methoxypyridin-4-yl)ethyl]-5′,6′-dihydro-1H-spiro[pyrrolidine-3,4′-pyrrolo[1,2-b]pyrazole]-1-carboxamide (Diastereomer 1 and Diastereomer 2)

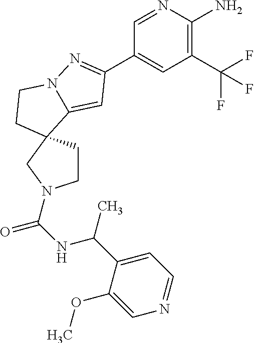 [SMILES: COc1cnccc1C(C)NC(=O)N1CC[C@@]2(CCn3nc(-c4cnc(N)c(C(F)(F)F)c4)cc32)C1]

The compound was prepared similarly to Example 789 using 5-[(3R)-5′,6′-dihydrospiro[pyrrolidine-3,4′-pyrrolo[1,2-b]pyrazol]-2′-yl]-3-(trifluoromethyl)pyridin-2-amine hydrogen chloride (1/1) (75.0 mg, 78% purity, 163 μmol) and 1-(3-methoxypyridin-4-yl)ethan-1-amine (27.2 mg, 179 μmol) to give 48.0 mg (95% purity, 56% yield) of the title compound.

LC-MS (Method 1): Rₜ=0.94 min; MS (ESIpos): m/z=503 [M+H]⁺

¹H-NMR (400 MHz, DMSO-d6) δ [ppm]: 1.272 (4.99), 1.277 (5.08), 1.289 (5.15), 1.293 (5.00), 2.066 (1.07), 2.074 (1.97), 2.083 (1.83), 2.093 (1.48), 2.518 (2.01), 2.523 (1.63), 2.567 (2.09), 3.444 (1.48), 3.469 (3.56), 3.486 (2.13), 3.495 (1.68), 3.511 (0.79), 3.521 (0.59), 3.547 (0.64), 3.562 (0.41), 3.601 (0.49), 3.915 (15.70), 3.919 (16.00), 4.179 (1.73), 4.195 (3.07), 4.211 (1.60), 5.062 (0.66), 5.071 (0.73), 5.081 (0.97), 5.088 (1.01), 5.099 (0.70), 5.107 (0.67), 6.469 (7.11), 6.473 (3.62), 6.519 (1.23), 6.530 (1.42), 6.539 (1.82), 6.551 (6.67), 7.253 (1.59), 7.265 (1.63), 7.330 (1.82), 7.342 (1.83), 8.015 (3.40), 8.021 (3.44), 8.119 (2.00), 8.131 (1.87), 8.165 (2.87), 8.177 (2.65), 8.262 (4.76), 8.267 (4.96), 8.589 (2.96), 8.592 (2.88).

### Example 791

### (3R)-2′-[6-amino-5-(trifluoromethyl)pyridin-3-yl]-N-[1-(pyridin-4-yl)cyclopropyl]-5′,6′-dihydrospiro[pyrrolidine-3,4′-pyrrolo[1,2-b]pyrazole]-1-carboxamide

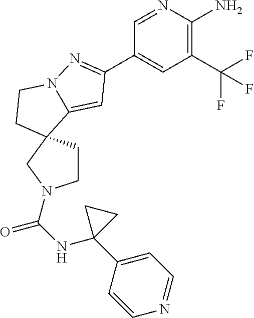 [SMILES: Nc1ncc(-c2cc3n(n2)CC[C@@]32CCN(C(=O)NC3(c4ccncc4)CC3)C2)cc1C(F)(F)F]

The compound was prepared similarly to Example 789 using 5-[(3R)-5′,6′-dihydrospiro[pyrrolidine-3,4′-pyrrolo[1,2-b]pyrazol]-2′-yl]-3-(trifluoromethyl)pyridin-2-amine hydrogen chloride (1/1) (105 mg, 78% purity, 228 μmol) and 1-(pyridin-4-yl)cyclopropan-1-amine (33.6 mg, 250 μmol) to give 58.0 mg (99% purity, 52% yield) of the title compound.

LC-MS (Method 1): Rₜ=0.89 min; MS (ESIpos): m/z=485 [M+H]⁺

¹H-NMR (400 MHz, DMSO-d6) δ [ppm]: 1.179 (0.44), 1.196 (0.51), 1.238 (0.59), 1.265 (6.93), 1.279 (6.64), 1.306 (0.45), 2.092 (1.92), 2.104 (2.18), 2.331 (2.79), 2.337 (1.21), 2.518 (16.00), 2.523 (11.14), 2.563 (4.07), 2.581 (2.80), 2.673 (2.78), 2.678 (1.23), 3.431 (0.61), 3.449 (1.54), 3.468 (10.24), 3.492 (0.83), 3.556 (0.76), 3.573 (1.40), 3.580 (0.89), 3.588 (1.01), 3.599 (0.91), 3.613 (0.52), 4.183 (2.92), 4.201 (4.63), 4.218 (2.76), 6.504 (10.71), 6.559 (7.26), 7.068 (8.07), 7.072 (4.99), 7.080 (4.96), 7.084 (8.10), 7.129 (4.10), 8.025 (4.16), 8.030 (4.20), 8.377 (9.16), 8.381 (5.27), 8.389 (4.99), 8.392 (8.65), 8.603 (3.96), 8.606 (3.84).

### Example 792

### (3R)-2′-{6-amino-5-[(1R)-1-(2-methoxyphenyl)ethoxy]pyridin-3-yl}-N-ethyl-5′,6′-dihydrospiro[pyrrolidine-3,4′-pyrrolo[1,2-b]pyrazole]-1-carboxamide

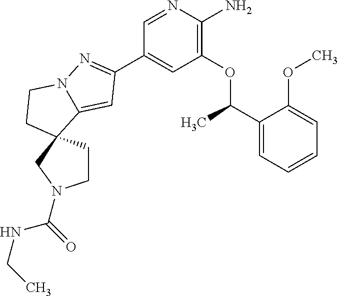 [SMILES: CCNC(=O)N1CC[C@@]2(CCn3nc(-c4cnc(N)c(O[C@H](C)c5ccccc5OC)c4)cc32)C1]

(3R)-1-(Ethylcarbamoyl)-5′,6′-dihydrospiro[pyrrolidine-3,4′-pyrrolo[1,2-b]pyrazol]-2′-yl trifluoromethanesulfonate (81.0 mg, 95% purity, 201 μmol), 3-[(1R)-1-(2-methoxyphenyl)ethoxy]-5-(4,4,5,5-tetramethyl-1,3,2-dioxaborolan-2-yl)pyridin-2-amine (142 mg, 63% purity, 242 μmol) and potassium phosphate (1.2 mL, 0.50 M, 600 μmol) were dissolved in 1,4-dioxane (20 mL) under nitrogen. XPhos Pd G2 (15.8 mg, 20.1 μmol) was added and the mixture was stirred for 3 h at 100° C. The cold reaction mixture was diluted with ethyl acetate and filtered over celite. The organic phase was washed with water and brine, dried over a hydrophobic filter and concentrated. The residue was purified twice by preparative HPLC to give 4.80 mg (100% purity, 5% yield) of the title compound.

LC-MS (Method 2): Rₜ=0.87 min; MS (ESIpos): m/z=477 [M+H]⁺

¹H NMR (DMSO-d₆, 400 MHz): δ (ppm) 7.82 (d, 1H), 7.39 (dd, 1H), 7.21-7.27 (m, 1H), 7.13 (d, 1H), 7.03 (d, 1H), 6.92 (td, 1H), 6.15 (t, 1H), 6.11 (s, 1H), 5.81 (s, 2H), 5.73 (q, 1H), 4.08-4.15 (m, 2H), 3.91 (s, 3H), 3.42-3.49 (m, 1H), 3.35-3.41 (m, 3H), 3.01-3.09 (m, 2H), 1.96-2.08 (m, 2H), 1.55 (d, 3H), 1.02 (t, 3H).

### Example 793

### 3-(1-{(3R)-2′-[6-amino-5-(trifluoromethyl)pyridin-3-yl]-5′,6′-dihydro-1H-spiro[pyrrolidine-3,4′-pyrrolo[1,2-b]pyrazol]-1-yl}propyl)benzonitrile (Diastereomer 1 and Diastereomer 2)

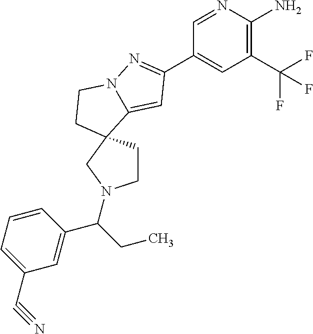 [SMILES: CCC(c1cccc(C#N)c1)N1CC[C@@]2(CCn3nc(-c4cnc(N)c(C(F)(F)F)c4)cc32)C1]

5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (100 mg, 95% purity, 264 μmol) and 3-propanoylbenzonitrile (63.1 mg, 396 μmol) were dissolved in methanol (2.8 mL). N,N-Diisopropylethylamine (180 μL, 1.1 mmol), titanium(IV) isopropoxide (230 μL, 790 μmol) and sodium cyanoborohydride (49.8 mg, 792 μmol) were added and the mixture was stirred over night at 60° C. The reaction mixture was allowed to cool down, diluted with water (0.5 mL) and stirred for 15 min. The suspension was filtered and the filter cake was washed with methanol. The filtrate was concentrated. The crude product was purified by preparative HPLC to give 54.0 mg (100% purity, 44% yield) of the title compound.

LC-MS (Method 1): Rₜ=1.48 min; MS (ESIpos): m/z=467 [M+H]⁺

¹H-NMR (400 MHz, DMSO-d6) δ [ppm]: 0.619 (6.70), 0.637 (15.32), 0.656 (7.05), 0.931 (0.91), 0.948 (0.92), 1.027 (0.45), 1.042 (0.47), 1.632 (0.61), 1.657 (1.00), 1.665 (1.24), 1.684 (1.04), 1.698 (0.68), 1.706 (0.65), 1.858 (0.70), 1.870 (1.05), 1.887 (1.29), 1.903 (1.17), 1.922 (0.86), 1.941 (0.64), 1.960 (1.16), 1.980 (2.26), 1.998 (3.31), 2.010 (2.00), 2.017 (1.94), 2.036 (0.62), 2.337 (0.97), 2.458 (1.79), 2.518 (16.00), 2.523 (14.89), 2.566 (6.47), 2.586 (2.50), 2.600 (1.09), 2.618 (0.51), 2.692 (1.48), 2.714 (3.48), 2.736 (2.37), 2.788 (0.45), 2.808 (0.83), 2.826 (0.87), 3.159 (5.56), 3.172 (5.91), 3.297 (2.84), 3.308 (3.97), 4.037 (0.50), 4.045 (1.03), 4.064 (2.17), 4.078 (3.68), 4.097 (5.99), 4.110 (2.85), 4.123 (0.88), 6.424 (9.10), 6.449 (11.15), 6.518 (10.08), 7.529 (1.65), 7.548 (4.43), 7.566 (3.90), 7.584 (1.76), 7.668 (3.55), 7.672 (3.72), 7.688 (2.75), 7.692 (2.70), 7.703 (2.01), 7.707 (2.90), 7.710 (1.90), 7.726 (3.76), 7.743 (4.68), 7.746 (4.86), 7.765 (4.28), 7.998 (3.01), 8.007 (4.40), 8.012 (3.68), 8.582 (2.80), 8.592 (3.69), 8.597 (3.36).

### Example 794

### 3-(1-{(3R)-2′-[6-amino-5-(trifluoromethyl)pyridin-3-yl]-5′,6′-dihydro-1H-spiro[pyrrolidine-3,4′-pyrrolo[1,2-b]pyrazol]-1-yl}propyl)benzonitrile (Diastereomer 1)

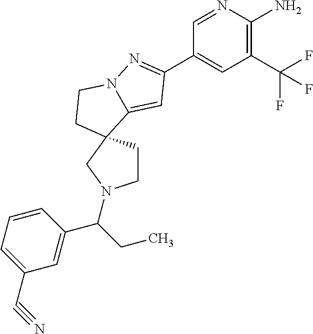 [SMILES: CCC(c1cccc(C#N)c1)N1CC[C@@]2(CCn3nc(-c4cnc(N)c(C(F)(F)F)c4)cc32)C1]

The title compound from Example 793 was separated into diastereomers by preparative chiral HPLC to give 16.9 mg (99% purity, 14% yield) of the title compound (diastereomer 1 Rₜ=8.4-10.14 min).

Preparative Chiral HPLC Method:

Instrument: PrepCon Labomatic HPLC-1; Column: YMC Amylose SA 10μ, 250×50; eluent A: hexane+0.1 vol % diethylamine; eluent B: ethanol; isocratic: 20% B; flow: 140 mL/min; temperature: 25° C.; UV: 280 nm

Analytical Chiral HPLC Method:

Instrument: Thermo Fisher UltiMate 3000; Column: YMC Amylose SA 3μ, 100×4.6; eluent A: hexane+0.1 vol % diethylamine; eluent B: ethanol; isocratic: 20% B; flow: 1.4 mL/min; temperature: 25° C.; UV: 280 nm

Analytical chiral HPLC: Rₜ=2.08 min

[α]²⁰_{D}: +78.2° (c=1.00, methanol)

LC-MS (Method 1): Rₜ=1.33 min; MS (ESIpos): m/z=467 [M+H]⁺

¹H-NMR (400 MHz, DMSO-d₆): δ [ppm]=0.64 (t, 3H), 1.66 (br dd, 1H), 1.84-1.96 (m, 1H), 1.97-2.05 (m, 2H), 2.54-2.60 (m, 4H), 2.82 (q, 1H), 4.01-4.14 (m, 2H), 6.42 (s, 1H), 6.52 (s, 2H), 7.52-7.60 (m, 1H), 7.68 (d, 1H), 7.71-7.76 (m, 2H), 8.00 (d, 1H), 8.58 (d, 1H).

### Example 795

### 3-(1-{(3R)-2′-[6-amino-5-(trifluoromethyl)pyridin-3-yl]-5′,6′-dihydro-1H-spiro[pyrrolidine-3,4′-pyrrolo[1,2-b]pyrazol]-1-yl}propyl)benzonitrile (Diastereomer 2)

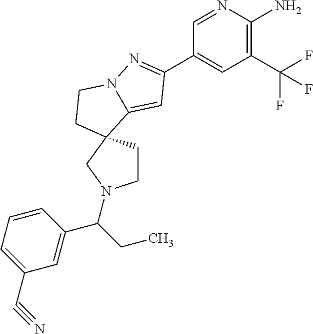 [SMILES: CCC(c1cccc(C#N)c1)N1CC[C@@]2(CCn3nc(-c4cnc(N)c(C(F)(F)F)c4)cc32)C1]

For the preparation of the diastereomeric mixture of the title compound see Example 793. The diastereomers were separated by preparative chiral HPLC (method see Example 794) to give 19.2 mg (100% purity, 16% yield) of the title compound (diastereomer 2 Rₜ=10.14-13.52 min).

Analytical chiral HPLC (method see Example 794): Rₜ=2.46 min.

[α]²⁰_{D}: +98.4° (c=1.00, methanol)

LC-MS (Method 1): Rₜ=1.34 min; MS (ESIpos): m/z=467 [M+H]⁺

¹H-NMR (400 MHz, DMSO-d6) δ [ppm]: 0.620 (3.07), 0.638 (7.08), 0.656 (3.48), 0.815 (0.44), 0.822 (0.41), 1.084 (0.54), 1.233 (1.68), 1.259 (0.94), 1.352 (0.85), 1.632 (0.43), 1.651 (0.59), 1.667 (0.70), 1.688 (0.76), 1.707 (0.53), 1.858 (0.50), 1.869 (0.60), 1.877 (0.63), 1.887 (0.72), 1.903 (0.57), 1.921 (0.46), 1.927 (0.45), 1.943 (0.47), 1.960 (0.94), 1.980 (1.10), 1.995 (1.12), 2.011 (1.18), 2.025 (0.98), 2.037 (0.47), 2.323 (1.11), 2.327 (1.55), 2.332 (1.18), 2.458 (0.74), 2.522 (16.00), 2.568 (4.68), 2.585 (3.04), 2.600 (2.01), 2.618 (1.21), 2.665 (1.56), 2.669 (2.23), 2.673 (1.95), 2.693 (1.44), 2.715 (3.05), 2.737 (2.09), 3.159 (2.27), 3.172 (2.41), 3.298 (1.20), 3.309 (1.66), 4.080 (1.75), 4.095 (3.77), 4.108 (1.46), 4.114 (1.87), 6.449 (7.92), 6.519 (5.41), 7.529 (1.28), 7.549 (3.10), 7.568 (2.10), 7.668 (2.15), 7.688 (1.67), 7.707 (2.30), 7.727 (1.92), 7.766 (3.55), 8.008 (2.93), 8.013 (3.04), 8.593 (2.79), 8.597 (2.82).

### Example 796

### (3S)-2′-(6-amino-5-cyanopyridin-3-yl)-N-[2-(3-chloropyridin-4-yl)propan-2-yl]-5′,6′-dihydrospiro[pyrrolidine-3,4′-pyrrolo[1,2-b]pyrazole]-1-carboxamide (Enantiomer 1)

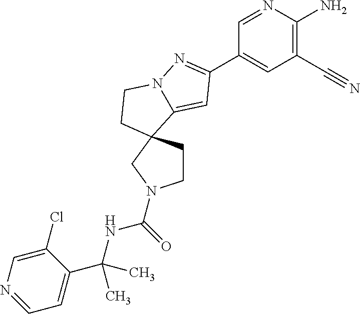 [SMILES: CC(C)(NC(=O)N1CC[C@]2(CCn3nc(-c4cnc(N)c(C#N)c4)cc32)C1)c1ccncc1Cl]

The racemic mixture of 2′-(6-amino-5-cyanopyridin-3-yl)-N-[2-(3-chloropyridin-4-yl)propan-2-yl]-5′,6′-dihydrospiro[pyrrolidine-3,4′-pyrrolo[1,2-b]pyrazole]-1-carboxamide was separated into enantiomers by preparative chiral HPLC to give 23.0 mg (95% purity, 33% yield) of the title compound (enantiomer 1 Rₜ=8.14-9.29 min).

Preparative Chiral HPLC Method:

Instrument: PrepCon Labomatic HPLC-2; Column: YMC Cellulose SC 5μ, 250×50; eluent A: methyl tert-butyl ether+0.1 vol % diethylamine; eluent B: ethanol; isocratic: 20% B; flow: 40 mL/min; temperature: 25° C.; UV: 254 nm

Analytical Chiral HPLC Method:

Instrument: Thermo Fisher UltiMate 3000; Column: YMC Cellulose SC 3μ, 100×4.6; eluent A: methyl tert-butyl ether+0.1 vol % diethylamine; eluent B: ethanol; isocratic: 20% B; flow: 1.4 mL/min; temperature: 25° C.; UV: 254 nm

Analytical chiral HPLC: Rₜ=3.89 min

[α]²⁰_{D}: −104.2° (c=1.00, DMSO)

LC-MS (Method 1): Rₜ=0.90 min; MS (ESIpos): m/z=477 [M+H]⁺

¹H-NMR (400 MHz, DMSO-d6) δ [ppm]: 0.796 (0.47), 0.813 (0.48), 0.820 (0.49), 0.903 (0.49), 1.230 (0.57), 1.653 (16.00), 2.049 (1.15), 2.057 (1.36), 2.074 (1.04), 2.475 (0.58), 2.518 (2.40), 2.523 (1.42), 3.382 (0.71), 3.408 (2.08), 3.421 (2.61), 3.446 (0.66), 3.496 (0.67), 3.504 (0.49), 3.511 (0.51), 3.521 (0.44), 4.162 (1.52), 4.180 (3.17), 4.198 (1.45), 6.331 (2.93), 6.431 (5.69), 7.004 (4.16), 7.426 (2.46), 7.439 (2.45), 8.127 (3.32), 8.133 (3.39), 8.390 (3.48), 8.403 (3.15), 8.419 (6.38), 8.601 (3.72), 8.607 (3.73).

### Example 797

### (3R)-2′-(6-amino-5-cyanopyridin-3-yl)-N-[2-(3-chloropyridin-4-yl)propan-2-yl]-5′,6′-dihydrospiro[pyrrolidine-3,4′-pyrrolo[1,2-b]pyrazole]-1-carboxamide (Enantiomer 2)

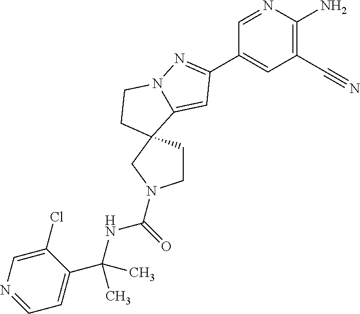 [SMILES: CC(C)(NC(=O)N1CC[C@@]2(CCn3nc(-c4cnc(N)c(C#N)c4)cc32)C1)c1ccncc1Cl]

The racemic mixture of 2′-(6-amino-5-cyanopyridin-3-yl)-N-[2-(3-chloropyridin-4-yl)propan-2-yl]-5′,6′-dihydrospiro[pyrrolidine-3,4′-pyrrolo[1,2-b]pyrazole]-1-carboxamide was separated into enantiomers by preparative chiral HPLC (method see Example 796) to give 20.0 mg (95% purity, 29% yield) of the title compound (enantiomer 2 Rₜ=9.29-11.84 min).

Analytical chiral HPLC (method see Example 796): Rₜ=5.43 min.

[α]²⁰_{D}: +131.5° (c=1.00, DMSO)

LC-MS (Method 1): Rₜ=0.90 min; MS (ESIpos): m/z=477 [M+H]⁺

¹H NMR (DMSO-d₆, 400 MHz): δ (ppm) 8.60 (d, 1H), 8.42 (s, 1H), 8.40 (d, 1H), 8.13 (d, 1H), 7.43 (d, 1H), 7.00 (s, 2H), 6.43 (s, 1H), 6.33 (s, 1H), 4.18 (t, 2H), 3.47-3.54 (m, 1H), 3.37-3.46 (m, 3H), 2.51-2.55 (m, 1H), 1.99-2.11 (m, 2H), 1.65 (s, 6H).

### Example 798

### (3R)-2′-{6-amino-5-[(1R)-1-(3-chlorophenyl)ethoxy]pyridin-3-yl}-N-(propan-2-yl)-5′,6′-dihydrospiro[pyrrolidine-3,4′-pyrrolo[1,2-b]pyrazole]-1-carboxamide

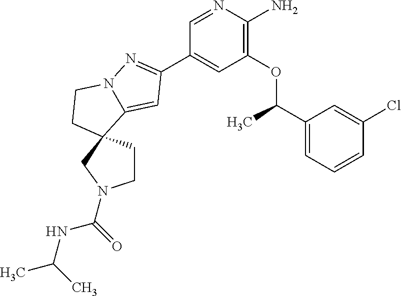 [SMILES: CC(C)NC(=O)N1CC[C@@]2(CCn3nc(-c4cnc(N)c(O[C@H](C)c5cccc(Cl)c5)c4)cc32)C1]

(3R)-1-[(Propan-2-yl)carbamoyl]-5′,6′-dihydrospiro[pyrrolidine-3,4′-pyrrolo[1,2-b]pyrazol]-2′-yl trifluoromethanesulfonate (78.6 mg, 79% purity, 157 μmol), 3-[(1R)-1-(3-chlorophenyl)ethoxy]-5-(4,4,5,5-tetramethyl-1,3,2-dioxaborolan-2-yl)pyridin-2-amine (80.0 mg, 88% purity, 188 μmol) and potassium phosphate (940 μL, 0.50 M, 470 μmol) were dissolved in 1,4-dioxane (12 mL) under nitrogen. XPhos Pd G2 (12.3 mg, 15.7 μmol) was added and the mixture was stirred for 3 h at 100° C. The cold reaction mixture was diluted with ethyl acetate and filtered over celite. The organic phase was washed with water and brine, dried over a hydrophobic filter and concentrated. The residue was purified by preparative HPLC to give 4.20 mg (94% purity, 5% yield) of the title compound.

LC-MS (Method 2): Rₜ=0.94 min; MS (ESIpos): m/z=495 [M+H]⁺

¹H NMR (DMSO-d₆, 400 MHz): δ (ppm) 7.86 (d, 1H), 7.58 (t, 1H), 7.43-7.47 (m, 1H), 7.38 (t, 1H), 7.29-7.33 (m, 1H), 7.27 (d, 1H), 6.25 (s, 1H), 5.91 (s, 2H), 5.81 (d, 1H), 5.64 (q, 1H), 4.13 (t, 2H), 3.70-3.80 (m, 1H), 3.45-3.52 (m, 1H), 3.37-3.42 (m, 3H), 1.96-2.08 (m, 2H), 1.56 (d, 3H), 1.07 (d, 3H), 1.06 (d, 3H).

### Example 799

### (3R)-2′-{6-amino-5-[1-(2,6-dichlorophenyl)ethoxy]pyridin-3-yl}-N-ethyl-5′,6′-dihydro-1H-spiro[pyrrolidine-3,4′-pyrrolo[1,2-b]pyrazole]-1-carboxamide (Diastereomer 1 and Diastereomer 2)

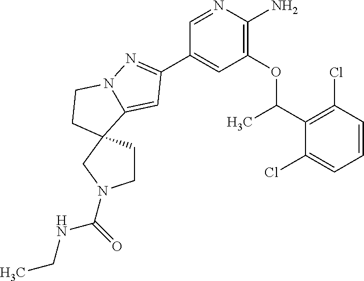 [SMILES: CCNC(=O)N1CC[C@@]2(CCn3nc(-c4cnc(N)c(OC(C)c5c(Cl)cccc5Cl)c4)cc32)C1]

To a solution of (rac)-3-{[1-(2,6-dichlorophenyl)ethyl]oxy}-5-(4,4,5,5-tetramethyl-1,3,2-dioxaborolan-2-yl)pyridin-2-amine (380 mg, 929 μmol) in 1,4-dioxane were added (3R)-1-(ethylcarbamoyl)-5′,6′-dihydrospiro[pyrrolidine-3,4′-pyrrolo[1,2-b]pyrazol]-2′-yl trifluoromethanesulfonate (391 mg, 1.02 mmol) and potassium phosphate (5.6 mL, 0.50 M, 2.8 mmol). The mixture was degassed with argon, XPhos Pd G2 (36.5 mg, 46.4 μmol) was added and stirred for 2 h at 100° C. The reaction mixture was allowed to cool down, diluted with water and extracted twice with ethyl acetate. The combined organic layers were washed with brine, dried over sodium sulfate, filtered and concentrated. The residue was purified by flash column chromatography (silica gel, hexane/ethyl acetate (0-100%) and dichloromethane/methanol (0-10%) gradient). The combined fractions were purified by preparative HPLC to give 38.0 mg (99% purity, 8% yield) of the title compound.

LC-MS (Method 1): Rₜ=1.10 min; MS (ESIpos): m/z=516 [M+H]⁺

¹H NMR (400 MHz, DMSO-d₆) δ ppm: 1.007 (2.09), 1.011 (2.53), 1.025 (4.78), 1.028 (5.62), 1.042 (2.41), 1.046 (2.66), 1.767 (5.50), 1.783 (5.60), 2.007 (0.85), 2.025 (1.46), 2.036 (1.25), 2.054 (0.48), 2.327 (0.40), 2.469 (1.65), 2.523 (3.36), 2.669 (0.41), 3.046 (0.97), 3.053 (1.10), 3.060 (1.14), 3.067 (1.39), 3.070 (1.26), 3.077 (1.08), 3.084 (1.10), 3.095 (0.40), 3.336 (16.00), 3.370 (4.41), 3.378 (2.82), 3.404 (0.70), 3.449 (0.66), 3.466 (0.64), 3.474 (0.56), 3.489 (0.42), 4.110 (1.49), 4.127 (2.51), 4.144 (1.52), 5.753 (3.61), 6.046 (1.35), 6.063 (1.39), 6.079 (0.46), 6.094 (3.82), 6.097 (3.89), 6.148 (0.60), 6.162 (1.17), 6.175 (0.64), 7.065 (2.48), 7.069 (2.63), 7.294 (0.77), 7.307 (0.76), 7.315 (1.30), 7.327 (1.22), 7.335 (1.32), 7.348 (1.10), 7.461 (2.91), 7.470 (2.63), 7.481 (2.19), 7.490 (1.87), 7.867 (3.09), 7.871 (3.35).

### Example 800

### (3R)-2′-{6-amino-5-[1-(2,6-dichlorophenyl)ethoxy]pyridin-3-yl}-N-ethyl-5′,6′-dihydro-1H-spiro[pyrrolidine-3,4′-pyrrolo[1,2-b]pyrazole]-1-carboxamide (Diastereomer 1)

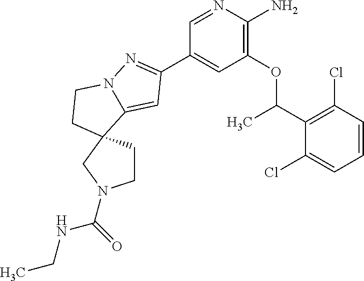 [SMILES: CCNC(=O)N1CC[C@@]2(CCn3nc(-c4cnc(N)c(OC(C)c5c(Cl)cccc5Cl)c4)cc32)C1]

The title compound from Example 799 was separated into diastereomers by preparative chiral HPLC to give 18.0 mg (95% purity, 45% yield) of the title compound (diastereomer 1 Rₜ=6.4-10.2 min).

Preparative Chiral HPLC Method:

Instrument: PrepCon Labomatic HPLC-3; Column: Chiralpak IF 5μ, 250×50; eluent A: methyl tert-butyl ether+0.1 vol % diethylamine; eluent B: ethanol+0.1 vol % diethylamine; isocratic: 40% B; flow:40 mL/min; temperature: 25° C.; UV: 280 nm

Analytical Chiral HPLC Method:

Instrument: Thermo Fisher UltiMate 3000; Column: Chiralpak IF 3μ, 100×4.6; eluent A: methyl tert-butyl ether+0.1 vol % diethylamine; eluent B: ethanol; isocratic: 40% B; flow: 1.4 mL/min; temperature: 25° C.; UV: 280 nm

Analytical chiral HPLC: Rₜ=2.29 min

[α]²⁰_{D}: +49.9° (c=1.00, DMSO)

LC-MS (Method 1): Rₜ=1.08 min; MS (ESIpos): m/z=515 [M+H]⁺

¹H NMR (DMSO-d₆, 400 MHz): δ (ppm) 7.87 (d, 1H), 7.48 (d, 2H), 7.30-7.35 (m, 1H), 7.07 (d, 1H), 6.16 (t, 1H), 6.10 (s, 1H), 6.05 (q, 1H), 5.75 (s, 2H), 4.13 (t, 2H), 3.42-3.49 (m, 1H), 3.35-3.41 (m, 3H), 3.02-3.10 (m, 2H), 1.98-2.06 (m, 2H), 1.77 (d, 3H), 1.02 (t, 3H).

### Example 801

### (3R)-2′-{6-amino-5-[1-(2,6-dichlorophenyl)ethoxy]pyridin-3-yl}-N-ethyl-5′,6′-dihydro-1H-spiro[pyrrolidine-3,4′-pyrrolo[1,2-b]pyrazole]-1-carboxamide (Diastereomer 2)

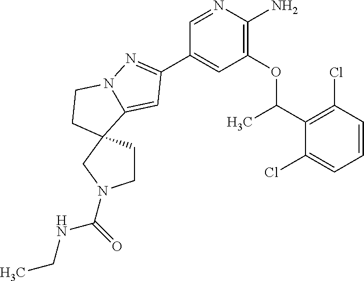 [SMILES: CCNC(=O)N1CC[C@@]2(CCn3nc(-c4cnc(N)c(OC(C)c5c(Cl)cccc5Cl)c4)cc32)C1]

For the preparation of the diastereomeric mixture of the title compound see Example 799. The diastereomers were separated by preparative chiral HPLC (method see Example 800) to give 20.0 mg (95% purity, 50% yield) of the title compound (diastereomer 2 Rₜ=14.1-19.9 min).

Analytical chiral HPLC (method see Example 800): Rₜ=5.23 min.

[α]²⁰_{D}: +5.6° (c=1.00, DMSO)

LC-MS (Method 1): Rₜ=1.08 min; MS (ESIpos): m/z=515 [M+H]⁺

¹H NMR (DMSO-d₆, 400 MHz): δ (ppm) 7.87 (d, 1H), 7.47 (d, 2H), 7.28-7.35 (m, 1H), 7.07 (d, 1H), 6.16 (t, 1H), 6.09 (s, 1H), 6.06 (q, 1H), 5.75 (s, 2H), 4.13 (t, 2H), 3.44-3.52 (m, 1H), 3.35-3.40 (m, 3H), 3.03-3.11 (m, 2H), 1.97-2.09 (m, 2H), 1.78 (d, 3H), 1.03 (t, 3H).

### Example 802

### 5-{(3R)-1-[1-(1H-pyrrol-2-yl)propyl]-5′,6′-dihydrospiro[pyrrolidine-3,4′-pyrrolo[1,2-b]pyrazol]-2′-yl}-3-(trifluoromethyl)pyridin-2-amine (Diastereomer 1 and Diastereomer 2)

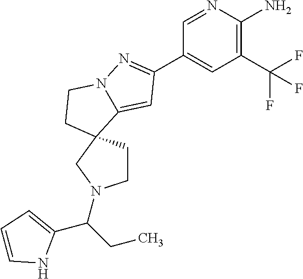 [SMILES: CCC(c1ccc[nH]1)N1CC[C@@]2(CCn3nc(-c4cnc(N)c(C(F)(F)F)c4)cc32)C1]

5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (100 mg, 85% purity, 236 μmol) and 1-(1H-pyrrol-2-yl)propan-1-one (43.6 mg, 354 μmol) were dissolved in methanol (2.5 mL). N,N-Diisopropylethylamine (160 μL, 950 μmol), titanium(IV) isopropoxide (210 μL, 710 μmol μmol) and sodium cyanoborohydride (44.5 mg, 709 μmol) were added and the mixture was stirred for 3 h at 60° C. The reaction mixture was allowed to reach rt, diluted with water (0.5 mL) and stirred for 15 min. The suspension was filtered and the filter cake was washed with methanol. The filtrate was concentrated. The crude product was purified by preparative HPLC to give 4.50 mg (96% purity, 4% yield) of the title compound.

LC-MS (Method 1): Rₜ=1.23 min; MS (ESIneg): m/z=429 [M−H]⁻

¹H-NMR (400 MHz, DMSO-d6) δ [ppm]: 0.736 (6.82), 0.751 (13.00), 0.755 (13.38), 0.769 (7.03), 0.861 (0.47), 0.951 (5.14), 1.035 (2.42), 1.052 (4.41), 1.070 (2.31), 1.233 (0.70), 1.674 (2.09), 1.691 (2.08), 1.709 (1.39), 1.805 (2.24), 1.824 (2.25), 1.838 (1.75), 1.857 (1.26), 1.907 (4.62), 1.926 (6.80), 1.944 (3.67), 2.327 (4.75), 2.421 (4.24), 2.565 (9.85), 2.580 (8.03), 2.603 (3.04), 2.669 (6.60), 2.688 (2.20), 2.702 (3.72), 2.725 (2.78), 2.778 (1.06), 2.796 (1.84), 2.815 (1.71), 2.969 (0.44), 3.159 (3.28), 3.171 (3.45), 3.421 (2.06), 3.435 (1.97), 3.439 (1.92), 3.452 (1.62), 3.469 (0.73), 3.566 (1.28), 4.033 (1.68), 4.054 (4.79), 4.074 (6.62), 4.089 (3.86), 4.113 (1.56), 4.348 (0.81), 4.361 (1.50), 4.374 (0.72), 5.758 (0.40), 5.846 (5.24), 5.929 (6.16), 6.237 (8.92), 6.298 (8.32), 6.512 (16.00), 6.637 (5.51), 7.988 (7.77), 8.567 (5.86), 10.581 (3.30).

### Example 803

### (3R)-2′-[6-amino-5-(trifluoromethyl)pyridin-3-yl]-N-[1-(2-methoxypyridin-4-yl)ethyl]-5′,6′-dihydro-1H-spiro[pyrrolidine-3,4′-pyrrolo[1,2-b]pyrazole]-1-carboxamide (Diastereomer 1 and Diastereomer 2)

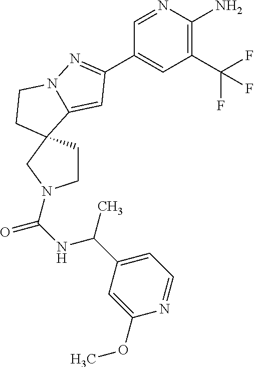 [SMILES: COc1cc(C(C)NC(=O)N2CC[C@@]3(CCn4nc(-c5cnc(N)c(C(F)(F)F)c5)cc43)C2)ccn1]

1-(2-Methoxypyridin-4-yl)ethan-1-amine (27.2 mg, 179 μmol), CDI (31.6 mg, 195 μmol) and N,N-diisopropylethylamine (110 μL, 650 μmol) were dissolved in DMF (1 mL) and stirred for 1 h at rt. A solution of 5-[(3R)-5′,6′-dihydrospiro[pyrrolidine-3,4′-pyrrolo[1,2-b]pyrazol]-2′-yl]-3-(trifluoromethyl)pyridin-2-amine hydrogen chloride (1/1) (75.0 mg, 78% purity, 163 μmol) in DMF (1 mL) was added and the mixture was stirred over night at rt. The reaction mixture was purified by preparative HPLC to give 40.0 mg (95% purity, 47% yield) of the title compound.

LC-MS (Method 1): Rₜ=0.99 min; MS (ESIpos): m/z=503 [M+H]⁺

¹H-NMR (400 MHz, DMSO-d6) δ [ppm]: 0.934 (0.84), 0.950 (0.82), 1.331 (4.51), 1.335 (4.22), 1.349 (4.58), 1.352 (4.17), 2.061 (0.89), 2.076 (1.67), 2.086 (1.31), 2.518 (1.89), 2.523 (1.62), 2.564 (1.96), 3.431 (1.09), 3.457 (2.55), 3.463 (3.21), 3.474 (1.17), 3.482 (1.25), 3.490 (0.56), 3.508 (0.48), 3.537 (0.54), 3.563 (0.51), 3.582 (0.57), 3.796 (10.93), 3.821 (16.00), 4.174 (1.86), 4.192 (3.18), 4.209 (1.79), 4.750 (0.87), 4.768 (1.31), 4.787 (0.86), 6.453 (2.75), 6.462 (5.27), 6.547 (4.76), 6.573 (0.93), 6.705 (1.65), 6.707 (1.80), 6.739 (2.06), 6.741 (2.23), 6.924 (0.98), 6.927 (0.97), 6.937 (1.02), 6.941 (1.00), 6.953 (1.18), 6.956 (1.16), 6.966 (1.23), 6.969 (1.17), 8.012 (2.73), 8.018 (2.82), 8.034 (1.52), 8.047 (1.51), 8.054 (2.02), 8.067 (1.90), 8.587 (2.71).

### Example 804

### (3R)-2′-[6-amino-5-(trifluoromethyl)pyridin-3-yl]-N-[1-(1-methyl-1H-pyrazol-5-yl)ethyl]-5′,6′-dihydrospiro[pyrrolidine-3,4′-pyrrolo[1,2-b]pyrazole]-1-carboxamide (Diastereomer 1)

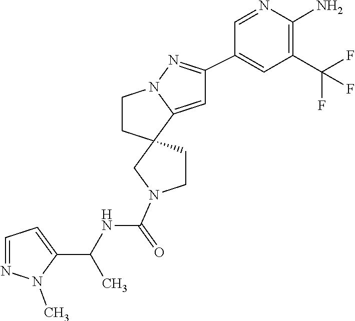 [SMILES: CC(NC(=O)N1CC[C@@]2(CCn3nc(-c4cnc(N)c(C(F)(F)F)c4)cc32)C1)c1ccnn1C]

1-(1-Methyl-1H-pyrazol-5-yl)ethanamine (41 mg, 330 μmol), CDI (58 mg, 360 μmol) and N,N-diisopropylethylamine (209 μL, 1.2 mmol) were dissolved in DMF (1 mL) and stirred for 1 h at room temperature. A solution of 5-[(3R)-5′,6′-dihydrospiro[pyrrolidine-3,4′-pyrrolo[1,2-b]pyrazol]-2′-yl]-3-(trifluoromethyl)pyridin-2-amine hydrogen chloride (1/1) (135 mg, 300 μmol) in DMF was added dropwise and the mixture was stirred over night at room temperature. The reaction mixture was poured into water and extracted with ethyl acetate. The combined organic layers were dried over a hydrophobic filter and concentrated. The residue was purified by flash column chromatography (silica gel, dichloromethane/methanol (0-8%) gradient to give 144 mg (98% purity, 99% yield) of the title compound as a mixture of diastereomers.

The mixture was separated into diastereomers by preparative chiral HPLC to give 36.1 mg (90% purity) of the title compound (diastereomer 1 Rₜ=4-5.4 min).

Preparative Chiral HPLC Method:

Instrument: Sepiatec: Prep SFC100; Column: Chiral Art Cellulose-SB 5μ, 250×50; eluent A: CO2; eluent B: 2-propanol; isocratic: 30% B; flow: 100 mL/min; temperature: 40° C.; BPR: 150 bar UV: 271 nm

Analytical Chiral HPLC Method:

Instrument: Waters Acquity UPC2 QDA; Column: Chiral Art Cellulose-SB 3μ, 100×4.6; eluent A: CO2; eluent B: 2-propanol+0.4 vol % diethylamine; isocratic: 30% B; flow: 4 mL/min; temperature: 40° C.; BPR: 100 bar; UV: 254 nm

Analytical chiral HPLC: Rₜ=1.07 min

[α]²⁰_{D}: +63.2° (c=1.00, methanol)

LC-MS (Method 1): Rₜ=0.91 min; MS (ESIpos): m/z=475 [M+H]⁺

¹H NMR (DMSO-d₆, 400 MHz): δ (ppm) 8.56-8.60 (m, 1H), 8.00 (d, 1H), 7.25 (d, 1H), 6.55 (s, 2H), 6.48 (d, 1H), 6.45 (s, 1H), 6.16 (d, 1H), 5.00 (quin, 1H), 4.18 (t, 2H), 3.75 (s, 3H), 3.55 (dt, 1H), 3.36-3.47 (m, 3H), 2.00-2.11 (m, 2H), 1.41 (d, 3H).

### Example 805

### (3R)-2′-[6-amino-5-(trifluoromethyl)pyridin-3-yl]-N-[1-(1-methyl-1H-pyrazol-5-yl)ethyl]-5′,6′-dihydrospiro[pyrrolidine-3,4′-pyrrolo[1,2-b]pyrazole]-1-carboxamide (Diastereomer 2)

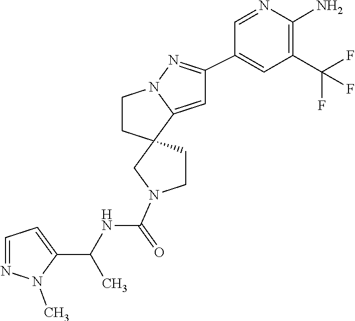 [SMILES: CC(NC(=O)N1CC[C@@]2(CCn3nc(-c4cnc(N)c(C(F)(F)F)c4)cc32)C1)c1ccnn1C]

For the preparation of the diastereomeric mixture of the title compound see Example 804. The diastereomers were separated by preparative chiral HPLC (method see Example 804) to give 35.8 mg (97% purity) of the title compound (diastereomer 2 Rₜ=8.5-10.5 min).

Analytical chiral HPLC (method see Example 804): Rₜ=2.43 min.

[α]²⁰_{D}: +76.8° (c=1.00, methanol)

LC-MS (Method 1): Rₜ=0.91 min; MS (ESIpos): m/z=475 [M+H]⁺

¹H NMR (DMSO-d₆, 400 MHz): δ (ppm) 8.58 (d, 1H), 8.00 (d, 1H), 7.27 (d, 1H), 6.55 (s, 2H), 6.46 (d, 1H), 6.44 (s, 1H), 6.19 (d, 1H), 4.95-5.05 (m, 1H), 4.18 (t, 2H), 3.77 (s, 3H), 3.48-3.56 (m, 1H), 3.39-3.48 (m, 3H), 2.00-2.11 (m, 2H), 1.40 (d, 3H).

### Example 806

### 3-(1-{(3R)-2′-[6-amino-5-(trifluoromethyl)pyridin-3-yl]-5′,6′-dihydro-1H-spiro[pyrrolidine-3,4′-pyrrolo[1,2-b]pyrazol]-1-yl}propyl)benzamide (Diastereomer 1 and Diastereomer 2)

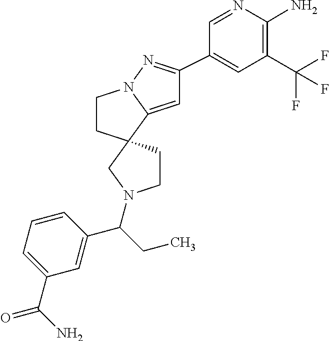 [SMILES: CCC(c1cccc(C(N)=O)c1)N1CC[C@@]2(CCn3nc(-c4cnc(N)c(C(F)(F)F)c4)cc32)C1]

5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (150 mg, 95% purity, 396 μmol) and 3-propanoylbenzamide (105 mg, 594 μmol) were dissolved in methanol (4.2 mL). N,N-Diisopropylethylamine (280 μL, 1.6 mmol), titanium(IV) isopropoxide (350 μL, 1.2 mmol) and sodium cyanoborohydride 74.7 mg, 1.19 mmol) were added and the mixture was stirred for 16 h at 60° C. The reaction mixture was allowed to reach rt, diluted with water (0.5 mL) and stirred for 15 min. The suspension was filtered and the filter cake was washed with methanol. The filtrate was concentrated. The crude product was purified by preparative HPLC to give 98.0 mg (100% purity, 51% yield) of the title compound.

LC-MS (Method 1): Rₜ=1.14 min; MS (ESIpos): m/z=486 [M+H]⁺

¹H-NMR (400 MHz, DMSO-d6) δ [ppm]: 0.628 (7.13), 0.646 (16.00), 0.664 (7.52), 0.930 (1.91), 0.947 (1.93), 1.640 (0.75), 1.658 (1.25), 1.674 (1.46), 1.691 (1.49), 1.710 (0.86), 1.870 (0.78), 1.888 (1.25), 1.899 (1.53), 1.916 (1.64), 1.932 (1.51), 1.949 (1.71), 1.976 (2.68), 1.992 (4.42), 2.011 (2.93), 2.043 (0.43), 2.323 (1.42), 2.327 (1.97), 2.331 (1.46), 2.451 (1.17), 2.518 (15.08), 2.522 (9.11), 2.558 (6.59), 2.565 (7.32), 2.584 (2.48), 2.599 (1.43), 2.616 (0.72), 2.659 (1.28), 2.665 (1.90), 2.669 (2.70), 2.673 (2.53), 2.678 (2.23), 2.693 (1.34), 2.715 (0.57), 2.740 (2.89), 2.763 (2.38), 2.812 (0.58), 2.827 (1.07), 2.849 (1.08), 2.867 (0.46), 3.159 (0.97), 3.172 (0.99), 3.241 (2.21), 3.247 (2.02), 3.253 (2.08), 3.262 (2.11), 4.025 (0.67), 4.034 (1.10), 4.052 (1.82), 4.076 (3.78), 4.092 (5.38), 4.111 (2.78), 6.418 (9.19), 6.439 (11.09), 6.514 (10.89), 7.342 (3.32), 7.373 (1.89), 7.392 (4.41), 7.407 (3.89), 7.411 (3.52), 7.426 (2.51), 7.455 (5.68), 7.474 (3.20), 7.737 (2.76), 7.748 (2.62), 7.752 (2.88), 7.756 (2.75), 7.766 (2.19), 7.794 (3.70), 7.814 (4.44), 7.968 (4.23), 7.998 (3.71), 8.007 (4.82), 8.013 (4.34), 8.577 (3.28), 8.580 (3.42), 8.588 (4.11), 8.592 (3.89).

### Example 807

### 3-(1-{(3R)-2′-[6-amino-5-(trifluoromethyl)pyridin-3-yl]-5′,6′-dihydrospiro[pyrrolidine-3,4′-pyrrolo[1,2-b]pyrazol]-1-yl}propyl)benzamide (Diastereomer 1)

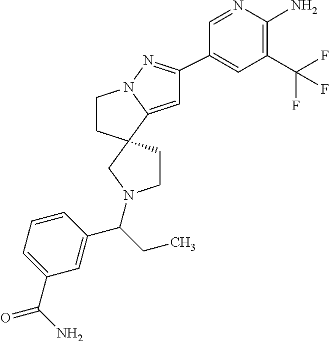 [SMILES: CCC(c1cccc(C(N)=O)c1)N1CC[C@@]2(CCn3nc(-c4cnc(N)c(C(F)(F)F)c4)cc32)C1]

The compound 3-(1-{(3R)-2′-[6-amino-5-(trifluoromethyl)pyridin-3-yl]-5′,6′-dihydrospiro[pyrrolidine-3,4′-pyrrolo[1,2-b]pyrazol]-1-yl}propyl)benzamide (diastereomer 1 and diastereomer 2, Example 806) was separated into diastereomers by preparative chiral HPLC to give 45.0 mg (98% purity, 23% yield) of the title compound (diastereomer 1 Rₜ=4.1-4.8 min).

Preparative Chiral HPLC Method:

Instrument: PrepCon Labomatic HPLC-3; Column: YMC Amylose SA 5μ, 250×50; eluent A: methyl tert-butyl ether+0.1 vol % diethylamine; eluent B: methanol; gradient; flow: 50 mL/min; temperature: 25° C.; UV: 280 nm

Analytical Chiral HPLC Method:

Instrument: Thermo Fisher UltiMate 3000; Column: YMC Amylose SA 3μ, 100×4.6; eluent A: methyl tert-butyl ether+0.1 vol % diethylamine; eluent B: methanol; gradient; flow: 1.4 mL/min; temperature: 25° C.; UV: 280 nm

Analytical chiral HPLC Rₜ=2.73 min

[α]²²_{D}: +70.4° (c=1.00, methanol)

LC-MS (Method 1): Rₜ=1.11 min; MS (ESIpos): m/z=485 [M+H]⁺

¹H NMR (400 MHz, DMSO-d₆) δ ppm 0.65 (t, 3H), 1.61-1.74 (m, 1H), 1.86-2.03 (m, 3H), 2.53-2.60 (m, 3H), 2.80-2.87 (m, 1H), 3.25 (dd, 1H), 4.00-4.13 (m, 2H), 6.42 (s, 1H), 6.51 (s, 2H), 7.35 (s, 1H), 7.38-7.43 (m, 1H), 7.45-7.48 (m, 1H), 7.76 (dt, 1H), 7.79 (s, 1H), 7.97 (s, 1H), 8.00 (d, 1H), 8.58 (d, 1H).

### Example 808

### 3-(1-{(3R)-2′-[6-amino-5-(trifluoromethyl)pyridin-3-yl]-5′,6′-dihydrospiro[pyrrolidine-3,4′-pyrrolo[1,2-b]pyrazol]-1-yl}propyl)benzamide (Diastereomer 2)

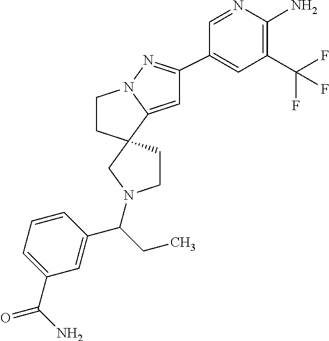 [SMILES: CCC(c1cccc(C(N)=O)c1)N1CC[C@@]2(CCn3nc(-c4cnc(N)c(C(F)(F)F)c4)cc32)C1]

The diastereomers were separated by preparative chiral HPLC (method see Example 807) to give 47.0 mg (96% purity, 24% yield) of the title compound (diastereomer 2 Rₜ=5.2-6.0 min).

Analytical chiral HPLC (method see Example 807): Rₜ=3.87 min.

[α]²²_{D}: +72.2° (c=1.00, methanol)

LC-MS (Method 1): Rₜ=1.12 min; MS (ESIpos): m/z=485 [M+H]⁺

¹H NMR (400 MHz, DMSO-d₆) δ ppm 0.65 (t, 3H), 1.62-1.74 (m, 1H), 1.85-2.05 (m, 3H), 2.53-2.62 (m, 2H), 2.65-2.72 (m, 1H), 2.75 (d, 1H), 3.25 (dd, 1H), 4.09 (t, 2H), 6.44 (s, 1H), 6.51 (s, 2H), 7.34 (s, 1H), 7.37-7.42 (m, 1H), 7.45-7.48 (m, 1H), 7.75 (dt, 1H), 7.81 (s, 1H), 7.96 (s, 1H), 8.01 (d, 1H), 8.59 (d, 1H).

### Example 809

### (3R)-2′-{6-amino-5-[(1R)-1-(1,3-thiazol-2-yl)ethoxy]pyridin-3-yl}-N-cyclobutyl-5′,6′-dihydrospiro[pyrrolidine-3,4′-pyrrolo[1,2-b]pyrazole]-1-carboxamide

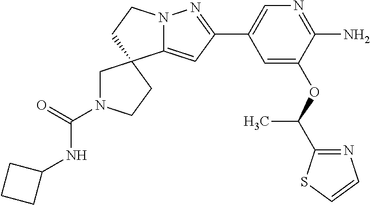 [SMILES: C[C@@H](Oc1cc(-c2cc3n(n2)CC[C@@]32CCN(C(=O)NC3CCC3)C2)cnc1N)c1nccs1]

5-(4,4,5,5-Tetramethyl-1,3,2-dioxaborolan-2-yl)-3-[(1R)-1-(1,3-thiazol-2-yl)ethoxy]pyridin-2-amine (264 mg, 49% purity, 374 μmol) was dissolved in 1,4-dioxane (2.1 mL). (3R)-1-(cyclobutylcarbamoyl)-5′,6′-dihydrospiro[pyrrolidine-3,4′-pyrrolo[1,2-b]pyrazol]-2′-yl trifluoromethanesulfonate (127 mg, 312 μmol), potassium phosphate (1.9 mL, 0.50 M, 940 μmol) and XPhos Pd G2 (24.5 mg, 31.2 μmol) were added and the mixture was stirred for 1 h at 100° C. The cold reaction mixture was diluted with dichloromethane. The organic phase was washed with water and brine, dried over a hydrophobic filter and concentrated. The residue was purified by preparative HPLC to give 54.0 mg (98% purity, 35% yield) of the title compound.

LC-MS (Method 1): Rₜ=0.91 min; MS (ESIpos): m/z=480 [M+H]⁺

¹H-NMR (400 MHz, DMSO-d6) δ [ppm]: 1.484 (0.72), 1.504 (1.44), 1.511 (1.18), 1.523 (1.24), 1.530 (2.75), 1.537 (1.24), 1.551 (2.39), 1.575 (1.78), 1.627 (0.49), 1.643 (0.48), 1.703 (15.59), 1.718 (15.89), 1.731 (0.90), 1.888 (1.26), 1.894 (1.21), 1.912 (2.26), 1.916 (2.16), 1.935 (2.22), 1.961 (1.30), 1.989 (0.64), 2.005 (1.68), 2.019 (2.62), 2.032 (3.36), 2.050 (1.55), 2.061 (1.52), 2.068 (1.48), 2.080 (2.62), 2.087 (3.09), 2.095 (2.21), 2.099 (2.06), 2.106 (2.56), 2.114 (1.87), 2.126 (0.87), 2.133 (0.66), 2.337 (0.51), 2.476 (4.46), 2.518 (7.31), 2.523 (5.10), 3.354 (1.53), 3.380 (13.87), 3.397 (2.81), 3.416 (1.07), 3.452 (1.04), 3.471 (1.90), 3.478 (1.10), 3.485 (1.33), 3.496 (1.09), 3.511 (0.62), 4.081 (0.98), 4.101 (1.86), 4.123 (5.33), 4.140 (6.64), 4.157 (3.52), 5.539 (0.46), 5.837 (8.98), 5.866 (1.22), 5.882 (4.08), 5.898 (4.07), 5.914 (1.08), 6.283 (16.00), 6.297 (3.39), 6.316 (3.17), 7.409 (6.10), 7.414 (6.01), 7.710 (9.93), 7.719 (10.91), 7.784 (0.61), 7.791 (12.47), 7.799 (9.10), 7.937 (8.84), 7.941 (8.54).

### Example 810

### (3R)-2′-{6-amino-5-[(1R)-1-(3-chlorophenyl)ethoxy]pyridin-3-yl}-N-ethyl-5′,6′-dihydrospiro[pyrrolidine-3,4′-pyrrolo[1,2-b]pyrazole]-1-carboxamide

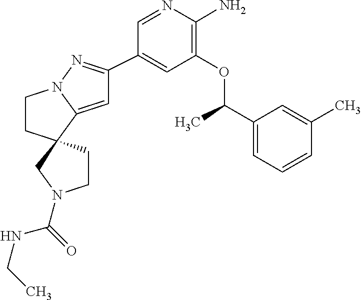 [SMILES: CCNC(=O)N1CC[C@@]2(CCn3nc(-c4cnc(N)c(O[C@H](C)c5cccc(C)c5)c4)cc32)C1]

(3R)-1-(Ethylcarbamoyl)-5′,6′-dihydrospiro[pyrrolidine-3,4′-pyrrolo[1,2-b]pyrazol]-2′-yl trifluoromethanesulfonate (66.8 mg, 95% purity, 166 μmol), 3-[(1R)-1-(3-chlorophenyl)ethoxy]-5-(4,4,5,5-tetramethyl-1,3,2-dioxaborolan-2-yl)pyridin-2-amine (91.0 mg, 82% purity, 199 μmol) and potassium phosphate (1000 μL, 0.50 M, 500 μmol) were dissolved in 1,4-dioxane (10 mL) under argon. XPhos Pd G2 (13.1 mg, 16.6 μmol) was added and the mixture was stirred for 4 h at 100° C. The cold reaction mixture was diluted with ethyl acetate and filtered over celite. The organic phase was washed with water and brine, dried over a hydrophobic filter and concentrated. The residue was purified twice by preparative HPLC to give 9.70 mg (94% purity, 11% yield) of the title compound.

[α]²⁰_{D}: +139.3° (c=1.00, methanol)

LC-MS (Method 2): Rₜ=0.88 min; MS (ESIpos): m/z=481 [M+H]⁺

¹H NMR (DMSO-d₆, 400 MHz): δ (ppm) 7.87 (d, 1H), 7.58 (t, 1H), 7.43-7.47 (m, 1H), 7.37 (t, 1H), 7.29-7.33 (m, 1H), 7.27 (d, 1H), 6.25 (s, 1H), 6.14 (t, 1H), 5.91 (s, 2H), 5.64 (q, 1H), 4.13 (t, 2H), 3.44-3.52 (m, 1H), 3.36-3.42 (m, 3H), 3.01-3.09 (m, 2H), 1.97-2.09 (m, 2H), 1.56 (d, 3H), 1.01 (t, 3H).

### Example 811

### (3R)-2′-[6-amino-5-(trifluoromethyl)pyridin-3-yl]-N-[1-(2-chloro-3-fluoropyridin-4-yl)ethyl]-5′,6′-dihydro-1H-spiro[pyrrolidine-3,4′-pyrrolo[1,2-b]pyrazole]-1-carboxamide (Diastereomer 1 and Diastereomer 2)

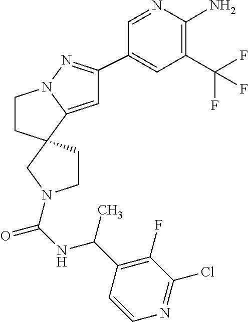 [SMILES: CC(NC(=O)N1CC[C@@]2(CCn3nc(-c4cnc(N)c(C(F)(F)F)c4)cc32)C1)c1ccnc(Cl)c1F]

1-(2-Chloro-3-fluoropyridin-4-yl)ethan-1-amine hydrogen chloride (1/1) (64.5 mg, 306 μmol) was dissolved in DMF (3 mL). CDI (54.1 mg, 334 μmol) and N,N-diisopropylethylamine (190 μL, 1.1 mmol) were added and stirred for 1 h at rt. 5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (125 mg, 80% purity, 278 μmol) was added and the mixture was stirred for 3 h at 60° C. The reaction mixture was allowed to cool down and purified by preparative HPLC followed by preparative TLC to give 8.00 mg (95% purity, 5% yield) of the title compound.

LC-MS (Method 1): Rₜ=1.03 min; MS (ESIpos): m/z=524 [M+H]⁺

¹H-NMR (400 MHz, DMSO-d6) δ [ppm]: 1.227 (1.15), 1.375 (9.30), 1.379 (9.22), 1.392 (9.56), 1.397 (8.93), 2.080 (3.69), 2.518 (2.07), 2.525 (3.83), 2.560 (3.73), 3.410 (0.66), 3.428 (2.88), 3.454 (5.77), 3.481 (5.12), 3.507 (2.59), 3.531 (1.32), 3.546 (0.86), 3.556 (0.72), 3.572 (0.80), 3.589 (1.02), 3.604 (0.76), 3.614 (0.77), 4.174 (3.51), 4.191 (6.19), 4.206 (3.23), 5.028 (0.44), 5.045 (1.75), 5.063 (2.61), 5.078 (1.70), 5.081 (1.69), 5.097 (0.42), 6.470 (16.00), 6.551 (11.59), 6.766 (2.24), 6.778 (2.40), 6.784 (2.52), 6.796 (1.93), 7.460 (1.47), 7.472 (2.83), 7.485 (1.51), 7.528 (1.67), 7.541 (3.21), 7.554 (1.70), 8.018 (6.08), 8.196 (3.69), 8.208 (3.52), 8.227 (5.69), 8.239 (5.46), 8.590 (6.45), 8.594 (6.29).

### Example 812

### (3R)-2′-[6-amino-5-(trifluoromethyl)pyridin-3-yl]-N-{[3-(methylsulfanyl)pyridin-4-yl]methyl}-5′,6′-dihydrospiro[pyrrolidine-3,4′-pyrrolo[1,2-b]pyrazole]-1-carboxamide

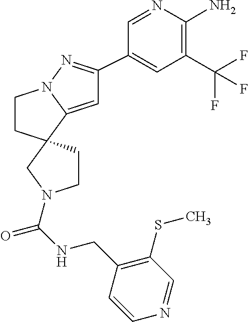 [SMILES: CSc1cnccc1CNC(=O)N1CC[C@@]2(CCn3nc(-c4cnc(N)c(C(F)(F)F)c4)cc32)C1]

1-[3-(Methylsulfanyl)pyridin-4-yl]methanamine (27.6 mg, 179 μmol) was dissolved in DMF (1 mL). CDI (31.6 mg, 195 μmol) and N,N-diisopropylethylamine (110 μL, 650 μmol) were added and stirred for 1 h at rt. A solution of 5-[(3R)-5′,6′-dihydrospiro[pyrrolidine-3,4′-pyrrolo[1,2-b]pyrazol]-2′-yl]-3-(trifluoromethyl)pyridin-2-amine hydrogen chloride (1/1) (75.0 mg, 78% purity, 163 μmol) in DMF (1 mL) was added and the mixture was stirred over night at rt. The reaction mixture was allowed to cool down and purified by preparative HPLC to give 23.0 mg (99% purity, 28% yield) of the title compound.

LC-MS (Method 1): Rₜ=0.94 min; MS (ESIpos): m/z=505 [M+H]⁺

¹H-NMR (400 MHz, DMSO-d6) δ [ppm]: 2.101 (0.55), 2.119 (0.96), 2.518 (1.39), 2.523 (0.99), 2.568 (16.00), 2.591 (1.15), 3.488 (0.74), 3.500 (4.13), 3.513 (0.86), 3.596 (0.61), 4.186 (1.17), 4.204 (1.91), 4.217 (2.23), 4.230 (1.93), 6.509 (4.55), 6.554 (2.84), 6.863 (0.44), 6.877 (0.88), 6.892 (0.42), 7.211 (1.28), 7.223 (1.29), 8.028 (1.65), 8.033 (1.64), 8.328 (2.50), 8.341 (2.40), 8.417 (3.92), 8.603 (1.53), 8.607 (1.51).

### Example 813

### (3R)-2′-{6-amino-5-[(1R)-1-(1,3-thiazol-2-yl)ethoxy]pyridin-3-yl}-N-ethyl-5′,6′-dihydrospiro[pyrrolidine-3,4′-pyrrolo[1,2-b]pyrazole]-1-carboxamide

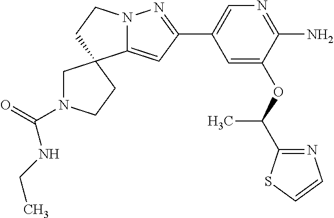 [SMILES: CCNC(=O)N1CC[C@@]2(CCn3nc(-c4cnc(N)c(O[C@H](C)c5nccs5)c4)cc32)C1]

5-(4,4,5,5-Tetramethyl-1,3,2-dioxaborolan-2-yl)-3-[(1R)-1-(1,3-thiazol-2-yl)ethoxy]pyridin-2-amine (247 mg, 46% purity, 327 μmol) was dissolved in 1,4-dioxane (1.8 mL) under argon. (3R)-1-(Ethylcarbamoyl)-5′,6′-dihydrospiro[pyrrolidine-3,4′-pyrrolo[1,2-b]pyrazol]-2′-yl trifluoromethanesulfonate (104 mg, 273 μmol), potassium phosphate (1.6 mL, 0.50 M, 820 μmol) and XPhos Pd G2 (10.7 mg, 13.6 μmol) were added and the mixture was stirred for 2 h at 100° C. The reaction mixture was allowed to cool down, diluted with dichloromethane and washed with water and brine. The organic phase was dried over a hydrophobic filter and concentrated. The crude product was purified by preparative HPLC to give 20.0 mg (99% purity, 16% yield) of the title compound.

LC-MS (Method 2): Rₜ=0.64 min; MS (ESIpos): m/z=454 [M+H]⁺

¹H-NMR (400 MHz, DMSO-d6): δ [ppm]=1.02 (t, 3H), 1.71 (d, 3H), 1.97-2.10 (m, 2H), 3.01-3.09 (m, 2H), 3.35-3.42 (m, 3H), 3.44-3.51 (m, 1H), 4.14 (t, 2H), 5.84 (s, 2H), 5.89 (q, 1H), 6.15 (t, 1H), 6.28 (s, 1H), 7.41 (d, 1H), 7.71 (d, 1H), 7.79 (d, 1H), 7.94 (d, 1H).

### Example 814

### (3R)-2′-[6-amino-5-(trifluoromethyl)pyridin-3-yl]-N-[1-(1,3-dimethyl-1H-pyrazol-4-yl)ethyl]-5′,6′-dihydrospiro[pyrrolidine-3,4′-pyrrolo[1,2-b]pyrazole]-1-carboxamide (Diastereomer 1)

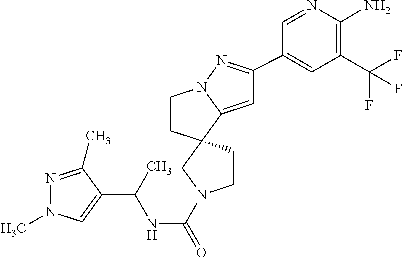 [SMILES: Cc1nn(C)cc1C(C)NC(=O)N1CC[C@@]2(CCn3nc(-c4cnc(N)c(C(F)(F)F)c4)cc32)C1]

1-(1,3-Dimethyl-1H-pyrazol-4-yl)ethanamine (46 mg, 330 μmol), CDI (58 mg, 360 μmol) and N,N-diisopropylethylamine (209 μL, 1.2 mmol) were dissolved in DMF (1 mL) and stirred for 1 h at rt. A solution of 5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (135 mg, 300 μmol) in DMF was added dropwise and the mixture was stirred over night at rt. The reaction mixture was poured into water and extracted with ethyl acetate. The combined organic layers were dried over a hydrophobic filter and concentrated. The residue was purified by flash column chromatography (silica gel, dichloromethane/methanol (0-8%) gradient to give 143 mg (97% yield) of the title compound as a mixture of diastereomers.

The mixture was separated into diastereomers by preparative chiral HPLC to give 36.6 mg (100% purity) of the title compound (diastereomer 1 Rₜ=8.4-10.3 min).

Preparative Chiral HPLC Method:

Instrument: Sepiatec: Prep SFC100; Column: Chiral Art Cellulose-SC 5μ, 250×50; eluent A: CO2; eluent B: methanol; isocratic: 30% B; flow: 100 mL/min; temperature: 40° C.; BPR: 150 bar UV: 271 nm

Analytical Chiral HPLC Method:

Instrument: Waters Acquity UPC2 QDA; Column: Chiral Art Cellulose-SC 3μ, 100×4.6; eluent A: CO2; eluent B: methanol+0.2 vol % aqueous ammonia (32%); isocratic: 30% B; flow: 4 mL/min; temperature: 40° C.; BPR: 100 bar; UV: 280 nm

Analytical chiral HPLC: Rₜ=2.24 min (ee value: >99.9%)

[α]²⁰_{D}: +58.8° (c=1.00, methanol)

LC-MS (Method 1): Rₜ=0.90 min; MS (ESIpos): m/z=489 [M+H]⁺

¹H NMR (DMSO-d₆, 400 MHz): δ (ppm) 8.58 (d, 1H), 8.00 (d, 1H), 7.45 (s, 1H), 6.55 (s, 2H), 6.44 (s, 1H), 6.09 (d, 1H), 4.70-4.79 (m, 1H), 4.18 (t, 2H), 3.69 (s, 3H), 3.36-3.53 (m, 4H), 1.98-2.11 (m, 5H), 1.31 (d, 3H).

### Example 815

### (3R)-2′-[6-amino-5-(trifluoromethyl)pyridin-3-yl]-N-[1-(1,3-dimethyl-1H-pyrazol-4-yl)ethyl]-5′,6′-dihydrospiro[pyrrolidine-3,4′-pyrrolo[1,2-b]pyrazole]-1-carboxamide (Diastereomer 2)

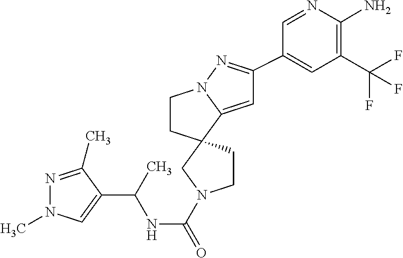 [SMILES: Cc1nn(C)cc1C(C)NC(=O)N1CC[C@@]2(CCn3nc(-c4cnc(N)c(C(F)(F)F)c4)cc32)C1]

For the preparation of the diastereomeric mixture of the title compound see Example 814. The diastereomers were separated by preparative chiral HPLC (method see Example 814) to give 36.8 mg (100% purity) of the title compound (diastereomer 2 Rₜ=14.3-18.0 min).

Analytical chiral HPLC (method see Example 814): Rₜ=3.90 min (ee value: >99.9%)

[α]²⁰_{D}: +67.4° (c=1.00, methanol)

LC-MS (Method 1): Rₜ=0.89 min; MS (ESIpos): m/z=489 [M+H]⁺

¹H NMR (DMSO-d₆, 400 MHz): δ (ppm) 8.58 (d, 1H), 8.01 (d, 1H), 7.43 (s, 1H), 6.54 (s, 2H), 6.44 (s, 1H), 6.10 (d, 1H), 4.71-4.80 (m, 1H), 4.17 (t, 2H), 3.67 (s, 3H), 3.54 (dt, 1H), 3.36-3.47 (m, 3H), 1.99-2.10 (m, 5H), 1.32 (d, 3H).

### Example 816

### (3R)-2′-{6-amino-5-[(1R)-1-(3-fluorophenyl)ethoxy]pyridin-3-yl}-N-(propan-2-yl)-5′,6′-dihydrospiro[pyrrolidine-3,4′-pyrrolo[1,2-b]pyrazole]-1-carboxamide

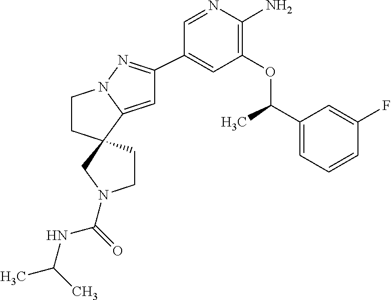 [SMILES: CC(C)NC(=O)N1CC[C@@]2(CCn3nc(-c4cnc(N)c(O[C@H](C)c5cccc(F)c5)c4)cc32)C1]

(3R)-1-[(Propan-2-yl)carbamoyl]-5′,6′-dihydrospiro[pyrrolidine-3,4′-pyrrolo[1,2-b]pyrazol]-2′-yl trifluoromethanesulfonate (69.0 mg, 79% purity, 137 μmol), 3-[(1R)-1-(3-fluorophenyl)ethoxy]-5-(4,4,5,5-tetramethyl-1,3,2-dioxaborolan-2-yl)pyridin-2-amine (104 mg, 71% purity, 206 μmol) and potassium phosphate (820 μL, 0.50 M, 410 μmol]) were dissolved in 1,4-dioxane (10 mL) under argon. XPhos Pd G2 (10.8 mg, 13.7 μmol) was added and the mixture was stirred for 2 h at 100° C. The cold reaction mixture was diluted with ethyl acetate and filtered over celite. The organic phase was washed with water and brine, dried over a hydrophobic filter and concentrated. The residue was purified by preparative HPLC to give 22.0 mg (98% purity, 33% yield) of the title compound.

[α]²⁰_{D}: +128.5° (c=1.00, methanol)

LC-MS (Method 2): Rₜ=0.89 min; MS (ESIpos): m/z=479 [M+H]⁺

¹H NMR (DMSO-d₆, 400 MHz): δ (ppm) 7.86 (d, 1H), 7.29-7.42 (m, 3H), 7.27 (d, 1H), 7.05-7.10 (m, 1H), 6.25 (s, 1H), 5.90 (s, 2H), 5.81 (d, 1H), 5.64 (q, 1H), 4.13 (t, 2H), 3.69-3.82 (m, 1H), 3.45-3.52 (m, 1H), 3.36-3.42 (m, 3H), 1.96-2.08 (m, 2H), 1.56 (d, 3H), 1.07 (d, 3H), 1.05 (d, 3H).

### Example 817

### (3R)-2′-[6-amino-5-(trifluoromethyl)pyridin-3-yl]-N-[1-(pyridin-2-yl)cyclopropyl]-5′,6′-dihydrospiro[pyrrolidine-3,4′-pyrrolo[1,2-b]pyrazole]-1-carboxamide

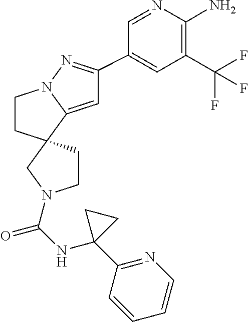 [SMILES: Nc1ncc(-c2cc3n(n2)CC[C@@]32CCN(C(=O)NC3(c4ccccn4)CC3)C2)cc1C(F)(F)F]

1-(Pyridin-2-yl)cyclopropan-1-amine (24.0 mg, 179 μmol), CDI (31.6 mg, 195 μmol) and N,N-diisopropylethylamine (110 μL, 650 μmol) were dissolved in DMF (2 mL) and stirred for 1 h at rt. A solution of 5-[(3R)-5′,6′-dihydrospiro[pyrrolidine-3,4′-pyrrolo[1,2-b]pyrazol]-2′-yl]-3-(trifluoromethyl)pyridin-2-amine hydrogen chloride (1/1) (75.0 mg, 78% purity, 163 μmol) in DMF was added and the mixture was stirred over night at rt. The reaction mixture was purified by preparative HPLC to give 32.0 mg (99% purity, 40% yield) of the title compound.

LC-MS (Method 1): Rₜ=0.94 min; MS (ESIpos): m/z=485 [M+H]⁺

¹H-NMR (400 MHz, DMSO-d6) δ [ppm]: 1.136 (2.60), 1.145 (6.72), 1.154 (6.84), 1.162 (3.19), 1.416 (3.22), 1.425 (7.62), 1.433 (6.41), 1.443 (2.89), 2.074 (0.94), 2.084 (1.63), 2.097 (2.58), 2.111 (3.01), 2.129 (1.33), 2.331 (2.04), 2.337 (0.89), 2.518 (11.95), 2.523 (8.37), 2.570 (5.59), 2.588 (3.84), 2.673 (2.00), 2.678 (0.88), 3.447 (0.76), 3.465 (2.01), 3.486 (13.95), 3.509 (1.09), 3.573 (0.98), 3.591 (1.88), 3.599 (1.21), 3.606 (1.35), 3.617 (1.18), 3.631 (0.68), 4.188 (3.93), 4.205 (6.14), 4.222 (3.76), 6.513 (16.00), 6.558 (9.86), 7.087 (3.18), 7.090 (3.01), 7.100 (3.10), 7.102 (3.42), 7.106 (3.32), 7.109 (3.14), 7.118 (3.65), 7.121 (3.86), 7.130 (5.74), 7.371 (2.93), 7.374 (5.06), 7.376 (2.91), 7.391 (3.30), 7.393 (5.19), 7.636 (2.83), 7.641 (3.12), 7.655 (3.75), 7.660 (3.63), 7.674 (2.24), 7.679 (2.30), 8.025 (5.64), 8.030 (5.71), 8.389 (3.44), 8.392 (3.88), 8.394 (3.72), 8.396 (3.49), 8.401 (3.54), 8.404 (3.94), 8.406 (3.48), 8.408 (3.15), 8.604 (5.32), 8.608 (5.22).

### Example 818

### 5-{(3R)-1-[1-(pyrimidin-5-yl)propyl]-5′,6′-dihydrospiro[pyrrolidine-3,4′-pyrrolo[1,2-b]pyrazol]-2′-yl}-3-(trifluoromethyl)pyridin-2-amine (Diastereomer 1 and Diastereomer 2)

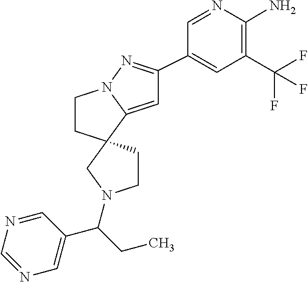 [SMILES: CCC(c1cncnc1)N1CC[C@@]2(CCn3nc(-c4cnc(N)c(C(F)(F)F)c4)cc32)C1]

5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1:1) (100 mg, 264 μmol) and 1-(pyrimidin-5-yl)propan-1-one (54 mg, 396 μmol) were dissolved in methanol (2.8 mL). N,N-Diisopropylethylamine (184 μL, 1.1 mmol), titanium(IV) isopropoxide (234 μL, 792 μmol) were added and stirred for 3 h at 60° C. Sodium cyanoborohydride (49.8 mg, 792 μmol) was added at rt and the mixture was stirred 3 h at 60° C. The reaction mixture was allowed to reach rt, diluted with water (0.5 mL) and stirred for 15 min. The suspension was filtered and the filter cake was washed with methanol. The filtrate was concentrated. The crude product was purified by preparative HPLC to give 67.0 mg (100% purity, 38% yield) of the title compound.

LC-MS (Method 1): Rₜ=1.06 min; MS (ESIpos): m/z=444 [M+H]⁺

¹H-NMR (400 MHz, DMSO-d₆): δ [ppm]=0.65-0.72 (m, 3H), 1.67-1.80 (m, 1H), 1.87-2.07 (m, 3H), 2.53-2.62 (m, 4H), 2.66-2.86 (m, 2H), 3.35-3.39 (m, 1H), 4.04-4.14 (m, 2H), 6.45 and 6.46 (2s, 1H), 6.52 (2br s, 2H), 7.99-8.03 (m, 1H), 8.58-8.61 (m, 1H), 8.74-8.78 (m, 2H), 9.09 and 9.11 (2s, 1H).

### Example 819

### 5-{(3R)-1-[1-(pyrimidin-5-yl)propyl]-5′,6′-dihydrospiro[pyrrolidine-3,4′-pyrrolo[1,2-b]pyrazol]-2′-yl}-3-(trifluoromethyl)pyridin-2-amine (Diastereomer 1)

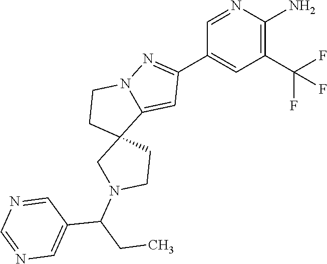 [SMILES: CCC(c1cncnc1)N1CC[C@@]2(CCn3nc(-c4cnc(N)c(C(F)(F)F)c4)cc32)C1]

The title compound from Example 818 was separated into diastereomers by preparative chiral HPLC to give 18.0 mg (95% purity, 45% yield) of the title compound (diastereomer 1 Rₜ=14.3-16.3 min).

Preparative Chiral HPLC Method:

Instrument: PrepCon Labomatic HPLC-2; Column: YMC Cellulose SC 5μ, 250×50; eluent A: hexane+0.1 vol % diethylamine; eluent B: ethanol; isocratic: 50% B; flow: 60 mL/min; temperature: 25° C.; UV: 280 nm

Analytical Chiral HPLC Method:

Instrument: Thermo Fisher UltiMate 3000; Column: YMC Cellulose SC 3μ, 100×4.6; eluent A: hexane+0.1 vol % diethylamine; eluent B: ethanol; isocratic: 50% B; flow: 1.4 mL/min; temperature: 25° C.; UV: 280 nm

Analytical chiral HPLC: Rₜ=1.96 min

[α]²⁰_{D}: +115.9° (c=1.00, methanol)

LC-MS (Method 1): Rₜ=1.06 min; MS (ESIpos): m/z=444 [M+H]⁺

¹H-NMR (400 MHz, DMSO-d₆): δ [ppm]=0.69 (t, 3H), 1.68-1.81 (m, 1H), 1.87-2.07 (m, 3H), 2.54-2.61 (m, 3H), 2.70 (d, 1H), 2.76 (td, 1H), 3.35-3.39 (m, 1H), 4.06-4.13 (m, 2H), 6.46 (s, 1H), 6.52 (s, 2H), 8.01 (d, 1H), 8.60 (d, 1H), 8.76 (s, 2H), 9.09 (s, 1H).

### Example 820

### 5-{(3R)-1-[1-(pyrimidin-5-yl)propyl]-5′,6′-dihydrospiro[pyrrolidine-3,4′-pyrrolo[1,2-b]pyrazol]-2′-yl}-3-(trifluoromethyl)pyridin-2-amine (Diastereomer 2)

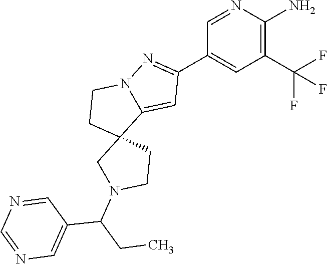 [SMILES: CCC(c1cncnc1)N1CC[C@@]2(CCn3nc(-c4cnc(N)c(C(F)(F)F)c4)cc32)C1]

For the preparation of the diastereomeric mixture of the title compound see Example 818. The diastereomers were separated by preparative chiral HPLC (method see Example 819) to give 31.0 mg (100% purity, 18% yield) of the title compound (diastereomer 2 Rₜ=18.4-20.5 min).

Analytical chiral HPLC (method see Example 819): Rₜ=2.48 min.

[α]²⁰_{D}: +62.9° (c=1.00, methanol)

LC-MS (Method 1): Rₜ=1.06 min; MS (ESIpos): m/z=444 [M+H]⁺

¹H-NMR (400 MHz, DMSO-d₆): δ [ppm]=0.68 (t, 3H), 1.67-1.79 (m, 1H), 1.88-2.07 (m, 3H), 2.54-2.62 (m, 4H), 2.78-2.85 (m, 1H), 3.35-3.40 (m, 1H), 4.02-4.14 (m, 2H), 6.45 (s, 1H), 6.52 (s, 2H), 8.01 (d, 1H), 8.59 (d, 1H), 8.77 (s, 2H), 9.11 (s, 1H).

### Example 821

### (3R)-2′-{6-amino-5-[(1R)-1-(1,3-thiazol-2-yl)ethoxy]pyridin-3-yl}-N-(propan-2-yl)-5′,6′-dihydrospiro[pyrrolidine-3,4′-pyrrolo[1,2-b]pyrazole]-1-carboxamide

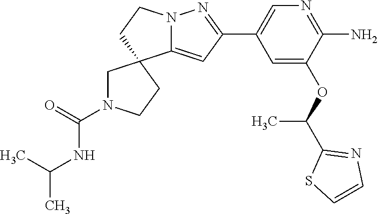 [SMILES: CC(C)NC(=O)N1CC[C@@]2(CCn3nc(-c4cnc(N)c(O[C@H](C)c5nccs5)c4)cc32)C1]

5-(4,4,5,5-Tetramethyl-1,3,2-dioxaborolan-2-yl)-3-[(1R)-1-(1,3-thiazol-2-yl)ethoxy]pyridin-2-amine (249 mg, 45% purity, 323 μmol) was dissolved in 1,4-dioxane (1.8 mL) under argon. (3R)-1-[(Propan-2-yl)carbamoyl]-5′,6′-dihydrospiro[pyrrolidine-3,4′-pyrrolo[1,2-b]pyrazol]-2′-yl trifluoromethanesulfonate (107 mg, 269 μmol), potassium phosphate (1.6 mL, 0.50 M, 810 μmol) and XPhos Pd G2 (21.2 mg, 26.9 μmol) were added and the mixture was stirred for 1 h at 100° C. The cold reaction mixture was diluted with dichloromethane. The organic phase was washed with water and brine, dried over a hydrophobic filter and concentrated. The residue was purified by preparative HPLC to give 34.3 mg (97% purity, 26% yield) of the title compound.

LC-MS (Method 2): Rₜ=0.73 min; MS (ESIpos): m/z=468 [M+H]⁺

¹H-NMR (400 MHz, DMSO-d6) δ [ppm]: 1.049 (14.46), 1.058 (16.00), 1.065 (15.63), 1.074 (15.05), 1.703 (13.88), 1.719 (13.91), 2.005 (1.53), 2.019 (2.45), 2.035 (3.21), 2.052 (1.29), 2.064 (0.60), 2.336 (0.91), 2.518 (12.63), 2.523 (8.48), 2.678 (0.85), 3.351 (1.21), 3.370 (2.16), 3.382 (12.65), 3.395 (2.74), 3.413 (0.97), 3.459 (0.92), 3.478 (1.60), 3.485 (0.99), 3.492 (1.16), 3.504 (0.98), 3.518 (0.56), 3.566 (1.02), 3.710 (0.42), 3.727 (1.16), 3.743 (1.70), 3.762 (1.71), 3.779 (1.13), 3.795 (0.40), 4.124 (3.21), 4.142 (5.63), 4.159 (3.09), 5.802 (3.02), 5.822 (3.13), 5.845 (6.56), 5.863 (1.44), 5.879 (3.57), 5.895 (3.54), 5.911 (0.98), 6.280 (12.74), 7.407 (5.33), 7.411 (5.14), 7.711 (8.17), 7.719 (9.24), 7.791 (9.70), 7.799 (8.43), 7.932 (7.24), 7.936 (7.10).

### Example 822

### (3R)-2′-[6-amino-5-(trifluoromethyl)pyridin-3-yl]-N-[(3-methylpyridin-4-yl)methyl]-5′,6′-dihydrospiro[pyrrolidine-3,4′-pyrrolo[1,2-b]pyrazole]-1-carboxamide

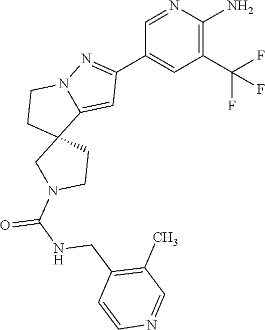 [SMILES: Cc1cnccc1CNC(=O)N1CC[C@@]2(CCn3nc(-c4cnc(N)c(C(F)(F)F)c4)cc32)C1]

1-(3-Methylpyridin-4-yl)methanamine hydrogen chloride (1/2) (36.7 mg, 95% purity, 179 μmol), CDI (31.6 mg, 195 μmol) and N,N-diisopropylethylamine (140 μL, 810 μmol) were dissolved in DMF (1 mL) and stirred for 1 h at rt. A solution of 5-[(3R)-5′,6′-dihydrospiro[pyrrolidine-3,4′-pyrrolo[1,2-b]pyrazol]-2′-yl]-3-(trifluoromethyl)pyridin-2-amine hydrogen chloride (1/1) (75.0 mg, 78% purity, 163 μmol) in DMF (1 mL) was added and the mixture was stirred over night at rt. The reaction mixture was purified by preparative HPLC to give 43.0 mg (95% purity, 53% yield) of the title compound.

LC-MS (Method 1): Rₜ=0.87 min; MS (ESIpos): m/z=473 [M+H]⁺

¹H-NMR (400 MHz, DMSO-d6) δ [ppm]: 0.937 (0.73), 0.953 (0.70), 2.093 (1.08), 2.111 (1.85), 2.120 (0.97), 2.128 (0.89), 2.255 (16.00), 2.518 (1.55), 2.523 (1.15), 2.565 (3.08), 2.582 (2.11), 3.457 (0.41), 3.475 (1.15), 3.489 (8.22), 3.499 (1.80), 3.518 (0.55), 3.566 (0.55), 3.584 (1.14), 3.592 (0.61), 3.599 (0.72), 3.610 (0.72), 4.181 (2.19), 4.200 (3.43), 4.217 (5.48), 4.232 (3.51), 6.494 (9.08), 6.551 (5.33), 6.801 (0.81), 6.816 (1.69), 6.831 (0.79), 7.190 (2.38), 7.202 (2.42), 8.024 (3.06), 8.029 (3.07), 8.293 (5.25), 8.321 (2.86), 8.333 (2.76), 8.597 (2.83), 8.602 (2.77).

### Example 823

### 5-{(3R)-1-[1-(6-methoxypyridin-2-yl)ethyl]-5′,6′-dihydrospiro[pyrrolidine-3,4′-pyrrolo[1,2-b]pyrazol]-2′-yl}-3-(trifluoromethyl)pyridin-2-amine (Diastereomer 1)

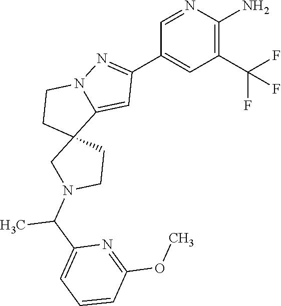 [SMILES: COc1cccc(C(C)N2CC[C@@]3(CCn4nc(-c5cnc(N)c(C(F)(F)F)c5)cc43)C2)n1]

5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (250 mg, 695 μmol) and 1-(6-methoxypyridin-2-yl)ethan-1-one (158 mg, 1.04 mmol) were dissolved in methanol (2.5 mL). N,N-Diisopropylethylamine (610 μL, 3.5 mmol), titanium(IV) isopropoxide (620 μL, 2.1 mmol) were added and stirred for 16 h at 60° C. The reaction mixture was allowed to cool down, sodium cyanoborohydride (131 mg, 2.08 mmol) was added and the mixture was stirred for 2 h at 60° C. The cold reaction mixture was allowed to cool down, diluted with water and stirred for 30 min. The suspension was diluted with methanol and filtered over celite. The filtrate was concentrated. The residue was diluted with dichloromethane/methanol (small amount of methanol) and washed with brine. The aqueous phase was extracted with dichloromethane/methanol. The combined organic layers were dried over sodium sulfate, filtered and concentrated. The crude product was purified by column chromatography (silica gel, dichloromethane/methanol (0-5%) gradient) followed by preparative TLC (dichloromethane/methanol 9:1 gradient) and preparative HPLC to give 49 mg of the diastereomeric mixture which was separated into diastereomers by preparative chiral HPLC to give 8.00 mg (75% purity, 2% yield) of the title compound.

LC-MS (Method 1): Rₜ=1.29 min; MS (ESIpos): m/z=460 [M+H]⁺

¹H-NMR (400 MHz, CHLOROFORM-d) δ [ppm]: 1.447 (2.20), 1.463 (2.11), 2.089 (0.46), 2.159 (0.48), 2.168 (0.52), 2.179 (0.68), 2.580 (0.59), 2.596 (0.53), 2.600 (0.61), 2.675 (0.44), 2.689 (0.48), 2.732 (0.42), 2.753 (0.44), 2.795 (0.91), 3.920 (16.00), 3.937 (3.67), 4.132 (0.66), 4.147 (0.72), 4.151 (0.76), 4.168 (0.77), 4.170 (0.86), 4.178 (0.41), 4.184 (0.85), 4.191 (0.79), 4.197 (0.65), 4.204 (0.73), 4.211 (0.41), 5.014 (2.37), 6.153 (1.16), 6.611 (1.12), 6.631 (1.15), 6.982 (1.29), 7.000 (1.39), 7.548 (1.26), 7.568 (1.44), 7.587 (0.89), 8.107 (1.46), 8.112 (1.50), 8.606 (1.39), 8.610 (1.38).

### Example 824

### 5-{(3R)-1-[1-(6-methoxypyridin-2-yl)ethyl]-5′,6′-dihydrospiro[pyrrolidine-3,4′-pyrrolo[1,2-b]pyrazol]-2′-yl}-3-(trifluoromethyl)pyridin-2-amine (Diastereomer 2)

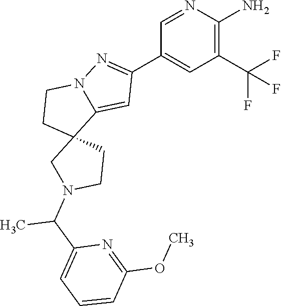 [SMILES: COc1cccc(C(C)N2CC[C@@]3(CCn4nc(-c5cnc(N)c(C(F)(F)F)c5)cc43)C2)n1]

For the preparation of the diastereomeric mixture of the title compound see Example 823. The diastereomers were separated by preparative HPLC to give 4.9 mg (98% purity, 2% yield) of the title compound.

LC-MS (Method 1): Rₜ=1.30 min; MS (ESIpos): m/z=459 [M+H]⁺

¹H-NMR (400 MHz, DMSO-d₆): δ [ppm]=0.68 (t, 3H), 1.67-1.79 (m, 1H), 1.88-2.07 (m, 3H), 2.54-2.62 (m, 4H), 2.78-2.85 (m, 1H), 3.35-3.40 (m, 1H), 4.02-4.14 (m, 2H), 6.45 (s, 1H), 6.52 (s, 2H), 8.01 (d, 1H), 8.59 (d, 1H), 8.77 (s, 2H), 9.11 (s, 1H).

### Example 825

### (3R)-2′-(6-amino-5-{1-[3-(3-hydroxy-3-methylbut-1-yn-1-yl)phenyl]ethoxy}pyridin-3-yl)-N-ethyl-5′,6′-dihydro-1H-spiro[pyrrolidine-3,4′-pyrrolo[1,2-b]pyrazole]-1-carboxamide (Diastereomer 1 and Diastereomer 2)

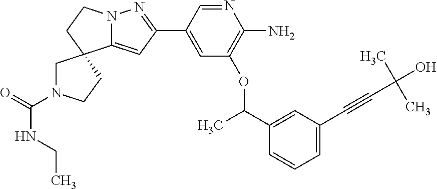 [SMILES: CCNC(=O)N1CC[C@@]2(CCn3nc(-c4cnc(N)c(OC(C)c5cccc(C#CC(C)(C)O)c5)c4)cc32)C1]

(3R)-1-(Ethylcarbamoyl)-5′,6′-dihydrospiro[pyrrolidine-3,4′-pyrrolo[1,2-b]pyrazol]-2′-yl trifluoromethanesulfonate (121 mg, 317 μmol), 4-[3-(1-{[2-amino-5-(4,4,5,5-tetramethyl-1,3,2-dioxaborolan-2-yl)pyridin-3-yl]oxy}ethyl)phenyl]-2-methylbut-3-yn-2-ol (174 mg, 412 μmol), potassium phosphate (1.9 mL) and XPhos Pd G2 (12.5 mg, 15.8 μmol) were combined in degassed 1,4-dioxane (5.2 mL). The mixture was stirred for 80 min at 100° C. The cold reaction mixture was diluted with dichloromethane and water. The phases were separated and the aqueous phase was extracted with dichloromethane/ethanol 9:1. The combined organic layers were concentrated. The residue was purified by flash column chromatography (silica gel, dichloromethane/methanol (0-10%) gradient). The combined fractions were purified by preparative HPLC to give 33.0 mg (90% purity, 18% yield) of the title compound.

[α]²⁰_{D}: +43.3° (c=1.00, methanol)

LC-MS (Method 1): Rₜ=1.00 min; MS (ESIpos): m/z=529 [M+H]⁺

¹H-NMR (400 MHz, DMSO-d₆): δ [ppm]=1.01 and 1.02 (2t, 3H), 1.44 (s, 6H), 1.52-1.59 (m, 3H), 1.95-2.09 (m, 2H), 3.00-3.09 (m, 2H), 3.35-3.42 (m, 3H), 3.43-3.52 (m, 1H), 4.12 (t, 2H), 5.47 (s, 1H), 5.52-5.64 (m, 1H), 5.79-5.92 (m, 2H), 6.14 (t, 1H), 6.20-6.25 (m, 1H), 7.20-7.57 (m, 5H), 7.84-7.86 (m, 1H).

### Example 826

### (3R)-2′-{6-amino-5-[(1R)-1-(pyridin-3-yl)ethoxy]pyridin-3-yl}-N-cyclobutyl-5′,6′-dihydrospiro[pyrrolidine-3,4′-pyrrolo[1,2-b]pyrazole]-1-carboxamide

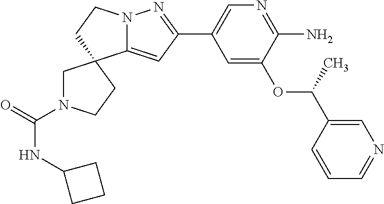 [SMILES: C[C@@H](Oc1cc(-c2cc3n(n2)CC[C@@]32CCN(C(=O)NC3CCC3)C2)cnc1N)c1cccnc1]

Cyclobutanamine (21.2 mg, 299 μmol) was dissolved in DMF (3.6 mL). CDI (44.4 mg, 274 μmol) and N,N-diisopropylethylamine (140 μL, 1000 μmol) was added and stirred for 1 h at rt. 5-[(3R)-5′,6′-Dihydrospiro[pyrrolidine-3,4′-pyrrolo[1,2-b]pyrazol]-2′-yl]-3-[(1R)-1-(pyridin-3-yl)ethoxy]pyridin-2-amine hydrogen chloride (1/1) (150 mg, 69% purity, 249 μmol) was added and the mixture was stirred for 1 h at 60° C. The reaction mixture was allowed to cool down and purified by preparative HPLC to give 54.7 mg (100% purity, 46% yield) of the title compound.

LC-MS (Method 1): Rₜ=0.85 min; MS (ESIpos): m/z=474 [M+H]⁺

¹H-NMR (400 MHz, DMSO-d₆): δ [ppm]=1.47-1.62 (m, 5H), 1.85-1.97 (m, 2H), 1.98-2.14 (m, 4H), 3.36-3.43 (m, 3H), 3.44-3.52 (m, 1H), 4.05-4.17 (m, 3H), 5.71 (q, 1H), 5.89 (s, 2H), 6.28 (s, 1H), 6.31 (d, 1H), 7.33 (d, 1H), 7.37 (dd, 1H), 7.87 (d, 1H), 7.91 (dt, 1H), 8.47 (dd, 1H), 8.71 (d, 1H).

### Example 827

### (3R)-2′-{6-amino-5-[1-(1,3-oxazol-2-yl)ethoxy]pyridin-3-yl}-N-ethyl-5′,6′-dihydro-1H-spiro[pyrrolidine-3,4′-pyrrolo[1,2-b]pyrazole]-1-carboxamide (Diastereomer 1 and Diastereomer 2)

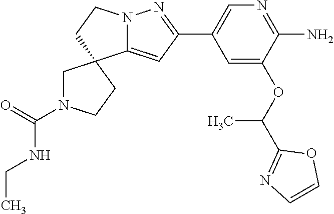 [SMILES: CCNC(=O)N1CC[C@@]2(CCn3nc(-c4cnc(N)c(OC(C)c5ncco5)c4)cc32)C1]

3-{[1-(1,3-Oxazol-2-yl)ethyl]oxy}-5-(4,4,5,5-tetramethyl-1,3,2-dioxaborolan-2-yl)pyridin-2-amine (178 mg, 48% purity, 258 μmol) was dissolved in 1,4-dioxane under nitrogen. (3R)-1-(Ethylcarbamoyl)-5′,6′-dihydrospiro[pyrrolidine-3,4′-pyrrolo[1,2-b]pyrazol]-2′-yl trifluoromethanesulfonate (82.2 mg, 215 μmol), potassium phosphate (1.3 mL, 0.50 M, 640 μmol) and XPhos Pd G2 (8.46 mg, 10.7 μmol) were added and the mixture was stirred for 1 h at 100° C. The cold reaction mixture was diluted with dichloromethane. The organic phase was washed with water and brine, dried over a hydrophobic filter and concentrated. The residue was purified by preparative HPLC to give 19.5 mg (100% purity, 21% yield) of the title compound.

LC-MS (Method 2): Rₜ=0.76 min; MS (ESIpos): m/z=438 [M+H]⁺

¹H-NMR (400 MHz, DMSO-d₆): δ [ppm]=1.02 (t, 3H), 1.70 (d, 3H), 1.99-2.11 (m, 2H), 3.01-3.09 (m, 2H), 3.36-3.43 (m, 3H), 3.45-3.52 (m, 1H), 4.16 (t, 2H), 5.67 (q, 1H), 5.81 (br s, 2H), 6.15 (t, 1H), 6.28 (s, 1H), 7.22-7.24 (m, 1H), 7.46 (d, 1H), 7.94 (d, 1H), 8.13 (d, 1H).

### Example 828

### (3R)-2′-{6-amino-5-[(1R)-1-(3-fluorophenyl)ethoxy]pyridin-3-yl}-N-ethyl-5′,6′-dihydrospiro[pyrrolidine-3,4′-pyrrolo[1,2-b]pyrazole]-1-carboxamide

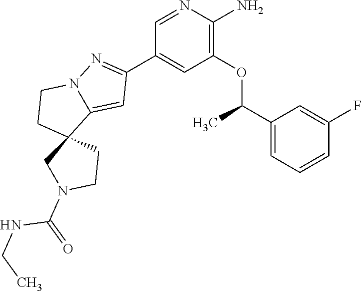 [SMILES: CCNC(=O)N1CC[C@@]2(CCn3nc(-c4cnc(N)c(O[C@H](C)c5cccc(F)c5)c4)cc32)C1]

(3R)-1-(Ethylcarbamoyl)-5′,6′-dihydrospiro[pyrrolidine-3,4′-pyrrolo[1,2-b]pyrazol]-2′-yl trifluoromethanesulfonate (101 mg, 95% purity, 250 μmol), 3-[(1R)-1-(3-fluorophenyl)ethoxy]-5-(4,4,5,5-tetramethyl-1,3,2-dioxaborolan-2-yl)pyridin-2-amine (170 mg, 79% purity, 375 μmol) and potassium phosphate (1.5 mL, 0.50 M, 750 μmol) were dissolved in 1,4-dioxane (15 mL) under argon. XPhos Pd G2 (19.7 mg, 25.0 μmol) was added and the mixture was stirred for 1 h at 100° C. The cold reaction mixture was diluted with ethyl acetate and filtered over celite. The organic phase was washed with water and brine, dried over a hydrophobic filter and concentrated. The residue was purified by preparative HPLC to give 24.1 mg (100% purity, 21% yield) of the title compound.

[α]²⁰_{D}: +121.6° (c=1.00, methanol)

LC-MS (Method 2): Rₜ=0.85 min; MS (ESIpos): m/z=465 [M+H]⁺

¹H NMR (DMSO-d₆, 400 MHz): δ (ppm) 7.86 (d, 1H), 7.30-7.43 (m, 4H), 7.05-7.12 (m, 1H), 6.29 (s, 2H), 6.15 (t, 1H), 5.70 (q, 1H), 4.14 (t, Hz, 2H), 3.43-3.51 (m, 1H), 3.36-3.42 (m, 3H), 3.00-3.10 (m, 2H), 1.97-2.09 (m, 2H), 1.57 (d, 3H), 1.01 (t, 3H).

### Example 829

### (3R)-2′-[6-amino-5-(6,7-dihydro-5H-cyclopenta[b]pyridin-7-yloxy)pyridin-3-yl]-N-ethyl-5′,6′-dihydro-1H-spiro[pyrrolidine-3,4′-pyrrolo[1,2-b]pyrazole]-1-carboxamide (Diastereomer 2)

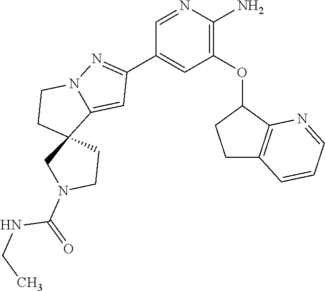 [SMILES: CCNC(=O)N1CC[C@@]2(CCn3nc(-c4cnc(N)c(OC5CCc6cccnc65)c4)cc32)C1]

(3R)-1-(Ethylcarbamoyl)-5′,6′-dihydrospiro[pyrrolidine-3,4′-pyrrolo[1,2-b]pyrazol]-2′-yl trifluoromethanesulfonate (126 mg, 329 μmol), {6-amino-5-[(6,7-dihydro-5H-cyclopenta[b]pyridin-7-yl)oxy]pyridin-3-yl}boronic acid (enantiomer 2) (223 mg, 40% purity, 329 μmol) and potassium phosphate (2.0 mL, 0.50 M, 990 μmol) were dissolved in 1,4-dioxane (5.4 mL) under argon. XPhos Pd G2 (12.9 mg, 16.5 μmol) was added and the mixture was stirred for 2 h at 100° C. The cold reaction mixture was diluted with ethyl acetate and washed with brine. The organic phase was dried over sodium sulfate, filtered and concentrated. The residue was purified by preparative HPLC followed by preparative TLC (dichloromethane/methanol 9:1 gradient) to give 22.2 mg (98% purity, 14% yield) of the title compound.

[α]²⁰_{D}: −13.7° (c=1.00, methanol)

LC-MS (Method 1): Rₜ=0.86 min; MS (ESIneg): m/z=458 [M−H]⁻

¹H-NMR (400 MHz, CHLOROFORM-d) δ [ppm]: 0.065 (0.72), 1.148 (5.63), 1.166 (12.88), 1.184 (5.83), 1.812 (1.06), 2.018 (0.54), 2.068 (0.56), 2.072 (0.56), 2.086 (0.95), 2.099 (0.77), 2.103 (0.72), 2.118 (0.56), 2.219 (0.56), 2.238 (1.07), 2.250 (0.55), 2.256 (0.71), 2.269 (0.76), 2.281 (0.46), 2.287 (0.50), 2.292 (0.51), 2.294 (0.54), 2.303 (0.56), 2.305 (0.51), 2.316 (0.92), 2.327 (0.96), 2.338 (0.64), 2.340 (0.66), 2.349 (0.64), 2.351 (0.58), 2.362 (0.50), 2.540 (0.93), 2.556 (1.04), 2.573 (1.56), 2.590 (0.80), 2.615 (0.82), 2.628 (0.59), 2.633 (1.77), 2.641 (0.66), 2.649 (1.34), 2.659 (0.70), 2.663 (1.18), 2.666 (1.45), 2.676 (0.58), 2.680 (1.09), 2.684 (0.96), 2.694 (0.51), 2.698 (0.52), 2.701 (0.47), 2.715 (0.44), 2.898 (0.44), 2.912 (0.45), 2.920 (0.41), 2.940 (0.67), 2.953 (0.67), 2.961 (0.60), 2.974 (0.52), 3.122 (0.56), 3.135 (0.60), 3.143 (0.59), 3.157 (0.58), 3.176 (0.42), 3.185 (0.41), 3.277 (0.79), 3.291 (1.01), 3.295 (2.53), 3.309 (2.70), 3.313 (2.65), 3.327 (2.60), 3.331 (0.99), 3.345 (0.80), 3.500 (1.07), 3.524 (2.26), 3.532 (1.04), 3.539 (0.76), 3.557 (1.40), 3.564 (2.96), 3.575 (0.66), 3.588 (1.37), 3.619 (0.54), 3.633 (0.68), 3.639 (0.78), 3.653 (0.66), 3.663 (0.46), 4.173 (0.63), 4.187 (1.16), 4.200 (0.61), 4.250 (2.65), 4.268 (5.09), 4.285 (2.49), 4.986 (3.97), 5.297 (13.38), 5.658 (1.22), 5.669 (1.31), 5.676 (1.34), 5.687 (1.21), 6.207 (9.16), 7.214 (1.58), 7.226 (1.63), 7.233 (1.75), 7.245 (1.81), 7.260 (16.00), 7.628 (1.49), 7.631 (1.54), 7.648 (1.39), 7.650 (1.39), 7.693 (3.24), 7.697 (3.34), 8.093 (4.53), 8.098 (4.32), 8.518 (1.48), 8.520 (1.50), 8.522 (1.56), 8.531 (1.52), 8.534 (1.47).

### Example 830

### 5-{(3R)-1-[cyclopropyl(5-fluoropyridin-3-yl)methyl]-5′,6′-dihydrospiro[pyrrolidine-3,4′-pyrrolo[1,2-b]pyrazol]-2′-yl}-3-(trifluoromethyl)pyridin-2-amine (Diastereomer 1 and Diastereomer 2)

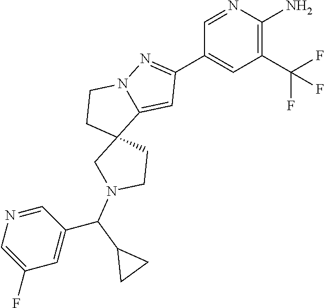 [SMILES: Nc1ncc(-c2cc3n(n2)CC[C@@]32CCN(C(c3cncc(F)c3)C3CC3)C2)cc1C(F)(F)F]

5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (150 mg, 95% purity, 396 μmol) and cyclopropyl(5-fluoropyridin-3-yl)methanone (98.1 mg, 594 μmol) were dissolved in methanol (4.2 mL). N,N-Diisopropylethylamine (280 μL, 1.6 mmol), titanium(IV) isopropoxide (350 μL, 1.2 mmol) were added and stirred for 1 h at 60° C. Sodium cyanoborohydride (74.7 mg, 1.19 mmol) was added and the mixture was stirred for 30 h at 60° C. and for 20 h at 80° C. The cooled down reaction mixture was diluted with water (0.5 mL) and stirred for 15 min. The suspension was filtered and the filter cake was washed with methanol. The filtrate was concentrated. The crude product was purified by preparative HPLC to give 81.0 mg (100% purity, 43% yield) of the title compound.

LC-MS (Method 1): Rₜ=1.23 min; MS (ESIpos): m/z=473 [M+H]⁺

¹H-NMR (400 MHz, DMSO-d6) δ [ppm]: 0.088 (0.65), 0.100 (0.99), 0.110 (1.13), 0.122 (0.91), 0.134 (0.66), 0.285 (0.92), 0.297 (1.15), 0.307 (0.97), 0.318 (0.69), 0.455 (0.63), 0.469 (0.84), 0.481 (0.92), 0.493 (0.90), 0.505 (0.75), 0.517 (0.56), 0.713 (1.04), 0.931 (1.32), 0.948 (1.32), 1.087 (0.74), 1.096 (0.90), 1.106 (0.88), 1.118 (0.81), 1.757 (0.73), 1.984 (0.44), 1.997 (0.77), 2.016 (1.18), 2.035 (1.51), 2.054 (1.36), 2.059 (1.37), 2.074 (0.87), 2.085 (0.43), 2.318 (0.91), 2.322 (1.96), 2.327 (2.78), 2.331 (2.05), 2.336 (0.97), 2.518 (16.00), 2.523 (9.98), 2.564 (2.56), 2.584 (2.71), 2.605 (2.58), 2.619 (2.53), 2.638 (1.79), 2.652 (0.84), 2.659 (1.29), 2.665 (2.20), 2.669 (3.19), 2.673 (2.21), 2.678 (1.00), 2.728 (1.43), 2.751 (1.09), 2.774 (0.47), 2.795 (0.94), 2.810 (0.82), 2.870 (1.85), 2.893 (1.73), 2.907 (0.57), 2.924 (0.53), 3.159 (0.40), 3.171 (0.42), 3.599 (0.60), 4.056 (0.62), 4.072 (1.20), 4.091 (2.22), 4.110 (2.69), 4.129 (1.32), 5.758 (0.84), 6.495 (5.64), 6.514 (9.83), 6.525 (5.05), 7.724 (1.40), 7.731 (1.77), 7.735 (1.57), 7.749 (1.44), 7.754 (1.67), 7.756 (1.68), 7.760 (1.51), 8.014 (1.89), 8.019 (3.97), 8.025 (2.63), 8.446 (7.60), 8.450 (5.14), 8.453 (5.36), 8.464 (2.59), 8.471 (2.41), 8.606 (3.68).

### Example 831

### (3R)-2′-{6-amino-5-[(1R)-1-(pyrimidin-2-yl)ethoxy]pyridin-3-yl}-N-(propan-2-yl)-5′,6′-dihydrospiro[pyrrolidine-3,4′-pyrrolo[1,2-b]pyrazole]-1-carboxamide

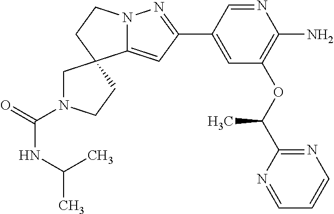 [SMILES: CC(C)NC(=O)N1CC[C@@]2(CCn3nc(-c4cnc(N)c(O[C@H](C)c5ncccn5)c4)cc32)C1]

(3R)-1-[(Propan-2-yl)carbamoyl]-5′,6′-dihydrospiro[pyrrolidine-3,4′-pyrrolo[1,2-b]pyrazol]-2′-yl trifluoromethanesulfonate (121 mg, 306 μmol), 3-[(1R)-1-(pyrimidin-2-yl)ethoxy]-5-(4,4,5,5-tetramethyl-1,3,2-dioxaborolan-2-yl)pyridin-2-amine (115 mg, 336 μmol), potassium phosphate (1.8 mL) and XPhos Pd G2 (12.0 mg, 15.3 μmol) were combined in degassed 1,4-dioxane (5.1 mL). The mixture was stirred for 80 min at 100° C. The cold reaction mixture was diluted with dichloromethane and water. The phases were separated and the aqueous phase was extracted with dichloromethane/ethanol 9:1. The combined organic layers were dried and concentrated. The residue was purified by flash column chromatography (silica gel NH2, dichloromethane/methanol (0-1%) gradient) to give 83.0 mg (90% purity, 53% yield) of the title compound.

LC-MS (Method 1): Rₜ=0.81 min; MS (ESIpos): m/z=463 [M+H]⁺

¹H-NMR (400 MHz, DMSO-d₆): δ [ppm]=1.04-1.08 (m, 6H), 1.68 (d, 3H), 1.96-2.08 (m, 2H), 2.44-2.48 (m, 2H), 3.35-3.41 (m, 3H), 3.44-3.52 (m, 1H), 3.70-3.81 (m, 1H), 4.09-4.15 (m, 2H), 5.51 (q, 1H), 5.78 (s, 2H), 5.81 (d, 1H), 6.19 (s, 1H), 7.20 (d, 1H), 7.44 (t, 1H), 7.87 (d, 1H), 8.83 (d, 2H).

### Example 832

### (3R)-2′-{6-amino-5-[(1R)-1-(4-fluorophenyl)ethoxy]pyridin-3-yl}-N-ethyl-5′,6′-dihydrospiro[pyrrolidine-3,4′-pyrrolo[1,2-b]pyrazole]-1-carboxamide

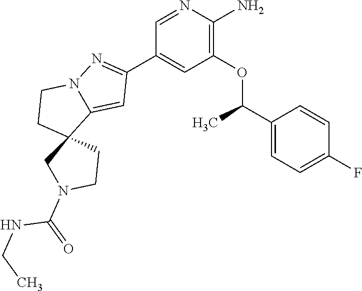 [SMILES: CCNC(=O)N1CC[C@@]2(CCn3nc(-c4cnc(N)c(O[C@H](C)c5ccc(F)cc5)c4)cc32)C1]

(3R)-1-(Ethylcarbamoyl)-5′,6′-dihydrospiro[pyrrolidine-3,4′-pyrrolo[1,2-b]pyrazol]-2′-yl trifluoromethanesulfonate (99.5 mg, 95% purity, 247 μmol), 3-[(1R)-1-(4-fluorophenyl)ethoxy]-5-(4,4,5,5-tetramethyl-1,3,2-dioxaborolan-2-yl)pyridin-2-amine (160 mg, 83% purity, 371 μmol) and potassium phosphate (1.5 mL, 0.50 M, 740 μmol) were dissolved in 1,4-dioxane (15 mL) under argon. XPhos Pd G2 (19.4 mg, 24.7 μmol) was added and the mixture was stirred for 3 h at 100° C. The reaction mixture was allowed to cool down and diluted with ethyl acetate (50 mL). It was filtered over celite and washed with ethyl acetate. The organic phase was washed with water and brine, dried over a hydrophobic filter and concentrated. The residue was purified by preparative HPLC to obtain 37.7 mg (100% purity, 33% yield) of the title compound.

[α]²⁰_{D}: +143.0° (c=1.00, methanol)

LC-MS (Method 1): Rₜ=0.98 min; MS (ESIpos): m/z=466 [M+H]⁺

¹H-NMR (400 MHz, DMSO-d6) δ [ppm]: 0.998 (6.62), 1.007 (0.85), 1.016 (16.00), 1.024 (0.81), 1.033 (6.99), 1.550 (8.05), 1.566 (8.07), 2.009 (1.07), 2.016 (1.09), 2.023 (1.55), 2.034 (2.13), 2.052 (0.96), 2.472 (1.87), 2.518 (2.27), 2.522 (1.51), 3.015 (0.77), 3.032 (2.48), 3.046 (2.69), 3.050 (2.69), 3.065 (2.43), 3.083 (0.73), 3.373 (6.79), 3.377 (6.27), 3.394 (2.14), 3.403 (1.08), 3.411 (0.90), 3.449 (0.72), 3.466 (1.19), 3.473 (0.72), 3.481 (0.84), 3.492 (0.70), 3.506 (0.40), 4.114 (2.11), 4.131 (3.66), 4.149 (2.16), 5.617 (0.46), 5.633 (1.65), 5.649 (1.66), 5.664 (0.46), 6.022 (1.04), 6.135 (0.99), 6.149 (1.95), 6.163 (0.97), 6.180 (0.43), 6.263 (9.07), 7.148 (3.02), 7.153 (0.98), 7.165 (1.22), 7.170 (6.01), 7.176 (1.18), 7.187 (1.02), 7.193 (3.27), 7.293 (3.32), 7.298 (3.37), 7.512 (2.90), 7.517 (1.22), 7.526 (3.12), 7.533 (2.96), 7.542 (1.06), 7.547 (2.64), 7.849 (4.55), 7.853 (4.48), 8.133 (0.56).

### Example 833

(3R)-2′-{6-amino-5-[(1R)-1-(4-fluorophenyl)ethoxy]pyridin-3-yl}-N-(propan-2-yl)-5′,6′-dihydrospiro[pyrrolidine-3,4′-pyrrolo[1,2-b]pyrazole]-1-carboxamide

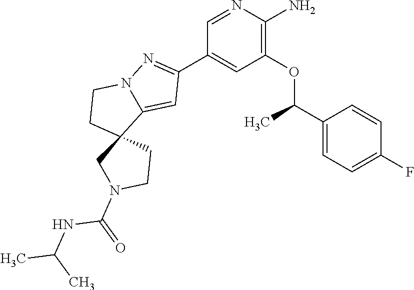 [SMILES: CC(C)NC(=O)N1CC[C@@]2(CCn3nc(-c4cnc(N)c(O[C@H](C)c5ccc(F)cc5)c4)cc32)C1]

(3R)-1-[(Propan-2-yl)carbamoyl]-5′,6′-dihydrospiro[pyrrolidine-3,4′-pyrrolo[1,2-b]pyrazol]-2′-yl trifluoromethanesulfonate (119 mg, 79% purity, 237 μmol), 3-[(1R)-1-(4-fluorophenyl)ethoxy]-5-(4,4,5,5-tetramethyl-1,3,2-dioxaborolan-2-yl)pyridin-2-amine (157 mg, 81% purity, 355 μmol) and potassium phosphate (1.4 mL, 0.50 M, 710 μmol) were dissolved in 1,4-dioxane (15 mL) under argon. XPhos Pd G2 (18.6 mg, 23.7 μmol) was added and the mixture was stirred for 2 h at 100° C. The reaction mixture was allowed to cool down and diluted with ethyl acetate (50 mL). It was filtered over celite and washed with ethyl acetate. The organic phase was washed with water and brine, dried over a hydrophobic filter and concentrated. The residue was purified by preparative HPLC affording 60.0 mg (100% purity, 53% yield) of the title compound.

[α]²⁰_{D}: +137.1° (c=1.00, methanol)

LC-MS (Method 1): Rₜ=1.07 min; MS (ESIpos): m/z=479 [M+H]⁺

¹H NMR (DMSO-d₆, 400 MHz): δ (ppm) 7.85 (d, 1H), 7.50-7.55 (m, 2H), 7.26 (d, 1H), 7.13-7.20 (m, 2H), 6.25 (s, 1H), 5.85 (s, 2H), 5.81 (d, 1H), 5.61 (q, 1H), 4.13 (t, 2H), 3.71-3.80 (m, 1H), 3.45-3.53 (m, 1H), 3.36-3.42 (m, 3H), 1.96-2.08 (m, 2H), 1.55 (d, 3H), 1.07 (d, 3H), 1.06 (d, 3H).

### Example 834

### 5-{(3R)-1-[1-(1,3-thiazol-2-yl)propyl]-5′,6′-dihydrospiro[pyrrolidine-3,4′-pyrrolo[1,2-b]pyrazol]-2′-yl}-3-(trifluoromethyl)pyridin-2-amine (Diastereomer 1 and Diastereomer 2)

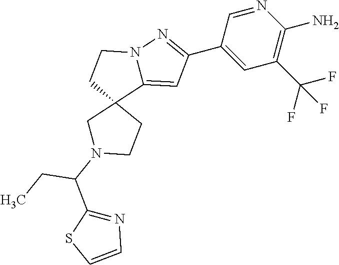 [SMILES: CCC(c1nccs1)N1CC[C@@]2(CCn3nc(-c4cnc(N)c(C(F)(F)F)c4)cc32)C1]

5-[(3R)-5′,6′-Dihydrospiro[pyrrolidine-3,4′-pyrrolo[1,2-b]pyrazol]-2′-yl]-3-(trifluoromethyl)pyridin-2-amine (200 mg, 619 μmol) and 1-(1,3-thiazol-2-yl)propan-1-one (87.3 mg, 619 μmol, CAS 43039-98-1) were dissolved in methanol (2.1 mL). N,N-Diisopropylethylamine (430 μL, 2.5 mmol) and titanium(IV) isopropoxide (550 μL, 1.9 mmol) were added and the mixture was stirred for 16 h at 60° C. The suspension was allowed to cool down, sodium cyanoborohydride (117 mg, 1.86 mmol) was added and stirred for 2 h at 60° C. 1-(1,3-Thiazol-2-yl)propan-1-one (43.6 mg, 309 μmol) and sodium cyanoborohydride (58 mg, 0.93 mmol) were added again and the mixture was stirred for 1 h at 60° C. The reaction mixture was allowed to cool down, diluted with water (1 mL) and stirred for 30 min. The suspension was filtered over celite and the filter cake was washed with methanol. The filtrate was concentrated. The residue was dissolved in dichloromethane/methanol (small amount of methanol) and twice with a saturated sodium bicarbonate solution. The aqueous phase was extracted with dichloromethane/methanol. The combined organic layers were dried over a hydrophobic filter and concentrated. The residue was purified twice by flash column chromatography (silica gel, dichloromethane/methanol (0-5%) gradient) to give 93.0 mg (100% purity, 34% yield) of the title compound.

LC-MS (Method 1): Rₜ=1.22 min; MS (ESIpos): m/z=449 [M+H]⁺

¹H-NMR (400 MHz, DMSO-d6) δ [ppm]: 0.787 (7.38), 0.806 (16.00), 0.824 (7.98), 0.851 (0.94), 1.173 (0.52), 1.233 (4.40), 1.254 (2.18), 1.720 (0.96), 1.738 (1.62), 1.755 (1.89), 1.772 (1.97), 1.791 (1.18), 1.931 (1.14), 1.947 (2.22), 1.973 (4.54), 1.979 (4.92), 1.990 (6.26), 2.008 (3.32), 2.048 (0.42), 2.327 (3.71), 2.522 (11.27), 2.560 (1.86), 2.575 (1.32), 2.592 (0.56), 2.669 (4.01), 2.674 (3.84), 2.699 (3.54), 2.711 (2.86), 2.734 (4.46), 2.754 (4.83), 2.776 (4.02), 2.798 (0.97), 2.814 (3.85), 2.837 (3.54), 2.843 (1.95), 2.858 (1.44), 2.881 (0.54), 2.918 (0.72), 2.935 (1.38), 2.957 (1.24), 2.974 (0.53), 3.835 (1.67), 3.849 (2.34), 3.857 (3.31), 3.866 (2.13), 3.879 (1.41), 4.068 (1.53), 4.077 (1.93), 4.086 (5.49), 4.103 (7.40), 4.120 (3.18), 5.760 (2.04), 6.368 (8.87), 6.399 (10.28), 6.521 (14.32), 7.673 (4.54), 7.681 (6.01), 7.689 (3.75), 7.697 (5.07), 7.735 (7.33), 7.742 (5.63), 7.998 (6.68), 8.004 (4.39), 8.573 (6.00).

### Example 835

### 5-{(3R)-1-[1-(1,3-thiazol-2-yl)propyl]-5′,6′-dihydrospiro[pyrrolidine-3,4′-pyrrolo[1,2-b]pyrazol]-2′-yl}-3-(trifluoromethyl)pyridin-2-amine (Diastereomer 1)

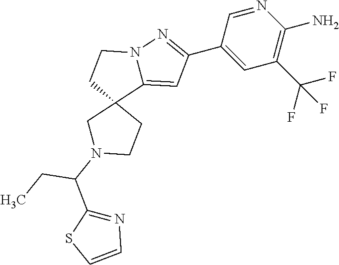 [SMILES: CCC(c1nccs1)N1CC[C@@]2(CCn3nc(-c4cnc(N)c(C(F)(F)F)c4)cc32)C1]

The title compound from Example 834 was separated into diastereomers by preparative chiral HPLC to give 28.0 mg (99% purity) of the title compound (diastereomer 1 Rₜ=6.4-7.2 min).

Preparative Chiral HPLC Method:

Instrument: PrepCon Labomatic HPLC-3; Column: YMC Amylose SB 5μ, 250×50; eluent A: hexane+0.1 vol % diethylamine; eluent B: ethanol; isocratic: 20% B; flow: 50 mL/min; temperature: 25° C.; UV: 280 nm

Analytical Chiral HPLC Method:

Instrument: Thermo Fisher UltiMate 3000; Column: YMC Amylose SB 3μ, 100×4.6; eluent A: hexane+0.1 vol % diethylamine; eluent B: ethanol; isocratic: 20% B; flow: 1.4 mL/min; temperature: 25° C.; UV: 280 nm

Analytical chiral HPLC: Rₜ=2.41 min

LC-MS (Method 1): Rₜ=1.22 min; MS (ESIpos): m/z=449 [M+H]⁺

¹H-NMR (400 MHz, CHLOROFORM-d) δ [ppm]: 0.772 (0.68), 0.783 (0.81), 0.798 (7.26), 0.816 (16.00), 0.835 (7.36), 1.185 (6.58), 1.215 (1.20), 1.231 (1.07), 1.746 (0.76), 1.764 (0.95), 1.767 (0.90), 1.779 (1.20), 1.786 (0.97), 1.798 (1.17), 1.801 (1.21), 1.820 (0.96), 1.920 (0.82), 1.941 (1.50), 1.946 (1.41), 1.952 (1.65), 1.959 (1.86), 1.969 (1.90), 1.973 (1.90), 1.978 (1.61), 1.989 (1.52), 1.998 (0.94), 2.012 (0.68), 2.043 (1.23), 2.056 (1.49), 2.065 (1.54), 2.077 (1.92), 2.088 (0.98), 2.096 (0.90), 2.109 (0.83), 2.438 (0.81), 2.454 (0.97), 2.459 (1.05), 2.471 (1.59), 2.486 (1.62), 2.491 (1.62), 2.506 (1.35), 2.558 (1.27), 2.573 (1.58), 2.578 (1.53), 2.592 (1.80), 2.706 (2.06), 2.729 (4.44), 2.740 (1.03), 2.762 (5.88), 2.784 (3.20), 2.801 (0.92), 2.922 (0.93), 2.934 (1.09), 2.944 (1.57), 2.956 (1.49), 2.965 (0.81), 2.978 (0.67), 3.795 (1.57), 3.807 (1.76), 3.816 (1.68), 3.830 (1.49), 4.033 (0.75), 4.048 (0.92), 4.053 (0.98), 4.060 (1.99), 4.068 (0.97), 4.075 (2.17), 4.080 (2.32), 4.092 (2.30), 4.095 (2.21), 4.106 (2.28), 4.113 (2.12), 4.120 (0.98), 4.127 (1.93), 4.133 (0.93), 4.140 (0.88), 4.154 (0.71), 4.917 (5.55), 5.233 (0.67), 6.077 (11.66), 7.252 (4.61), 7.260 (4.78), 7.658 (6.77), 7.666 (6.41), 8.029 (4.07), 8.033 (4.06), 8.521 (3.87), 8.525 (3.76).

### Example 836

### 5-{(3R)-1-[1-(1,3-thiazol-2-yl)propyl]-5′,6′-dihydrospiro[pyrrolidine-3,4′-pyrrolo[1,2-b]pyrazol]-2′-yl}-3-(trifluoromethyl)pyridin-2-amine (Diastereomer 2)

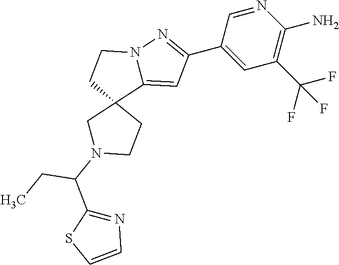 [SMILES: CCC(c1nccs1)N1CC[C@@]2(CCn3nc(-c4cnc(N)c(C(F)(F)F)c4)cc32)C1]

For the preparation of the diastereomeric mixture of the title compound see Example 834 The diastereomers were separated by preparative chiral HPLC (method see Example 835) to give 26.0 mg (99% purity) of the title compound (diastereomer 2 Rₜ=7.8-8.9 min).

Analytical chiral HPLC (method see Example 835): Rₜ=2.99 min.

LC-MS (Method 1): Rₜ=1.22 min; MS (ESIpos): m/z=449 [M+H]⁺

¹H-NMR (400 MHz, CHLOROFORM-d) δ [ppm]: 0.798 (6.93), 0.817 (16.00), 0.835 (7.38), 1.185 (2.68), 1.231 (0.64), 1.330 (0.48), 1.755 (0.77), 1.773 (0.92), 1.776 (0.90), 1.788 (1.18), 1.794 (0.95), 1.807 (1.16), 1.810 (1.27), 1.829 (1.03), 1.889 (0.78), 1.903 (0.93), 1.910 (1.01), 1.922 (1.82), 1.935 (1.84), 1.938 (1.64), 1.942 (1.67), 1.957 (2.12), 1.971 (1.41), 1.986 (0.89), 1.991 (0.88), 2.004 (0.70), 2.060 (1.22), 2.076 (1.50), 2.081 (1.60), 2.092 (1.10), 2.096 (1.53), 2.107 (1.13), 2.113 (1.08), 2.129 (0.97), 2.430 (0.84), 2.445 (0.98), 2.450 (1.12), 2.463 (1.72), 2.478 (1.61), 2.483 (1.75), 2.498 (1.40), 2.554 (1.39), 2.570 (1.79), 2.574 (1.62), 2.589 (1.79), 2.602 (1.15), 2.726 (0.88), 2.744 (2.54), 2.767 (6.54), 2.784 (6.65), 2.806 (1.75), 2.892 (1.04), 2.906 (1.16), 2.914 (1.77), 2.928 (1.67), 2.936 (0.97), 2.950 (0.75), 3.786 (1.67), 3.800 (1.86), 3.808 (1.79), 3.821 (1.63), 4.053 (0.55), 4.068 (0.71), 4.073 (0.61), 4.080 (2.29), 4.088 (0.79), 4.095 (4.29), 4.100 (2.92), 4.111 (2.91), 4.116 (4.08), 4.123 (0.79), 4.131 (2.19), 4.139 (0.61), 4.143 (0.68), 4.158 (0.52), 4.909 (5.42), 5.233 (0.46), 6.123 (14.12), 6.929 (0.50), 7.235 (4.60), 7.243 (4.79), 7.451 (0.50), 7.653 (7.28), 7.661 (6.95), 8.038 (3.90), 8.042 (3.95), 8.534 (3.79), 8.537 (3.72).

### Example 837

### (3R)-2′-{6-amino-5-[(1R)-1-(4-chlorophenyl)ethoxy]pyridin-3-yl}-N-ethyl-5′,6′-dihydrospiro[pyrrolidine-3,4′-pyrrolo[1,2-b]pyrazole]-1-carboxamide

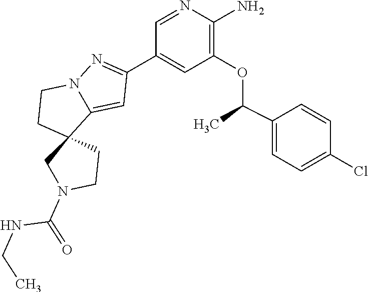 [SMILES: CCNC(=O)N1CC[C@@]2(CCn3nc(-c4cnc(N)c(O[C@H](C)c5ccc(Cl)cc5)c4)cc32)C1]

(3R)-1-(Ethylcarbamoyl)-5′,6′-dihydrospiro[pyrrolidine-3,4′-pyrrolo[1,2-b]pyrazol]-2′-yl trifluoromethanesulfonate (66.4 mg, 95% purity, 165 μmol), 3-[(1R)-1-(4-chlorophenyl)ethoxy]-5-(4,4,5,5-tetramethyl-1,3,2-dioxaborolan-2-yl)pyridin-2-amine (122 mg, 76% purity, 247 μmol) and potassium phosphate (990 μL, 0.50 M, 740 μmol) were dissolved in 1,4-dioxane (10 mL) under argon. XPhos Pd G2 (13.0 mg, 16.5 μmol) was added and the mixture was stirred for 4 h at 100° C. The reaction mixture was allowed to cool down and diluted with ethyl acetate (50 mL). It was filtered over celite and washed with ethyl acetate. The organic phase was washed with water and brine, dried over a hydrophobic filter and concentrated. The residue was purified by preparative HPLC to obtain 6.8 mg (95% purity, 8% yield) of the title compound.

[α]²⁰_{D}: +189.0° (c=1.00, methanol)

LC-MS (Method 1): Rₜ=1.04 min; MS (ESIpos): m/z=481 [M+H]⁺

¹H NMR (DMSO-d₆, 400 MHz): δ (ppm) 7.86 (d, 1H), 7.48-7.52 (m, 2H), 7.38-7.42 (m, 2H), 7.24 (d, 1H), 6.25 (s, 1H), 6.15 (t, 1H), 5.86 (s, 2H), 5.62 (q, 1H), 4.13 (t, 2H), 3.44-3.52 (m, 1H), 3.36-3.42 (m, 3H), 3.00-3.09 (m, 2H), 1.96-2.07 (m, 2H), 1.55 (d, 3H), 1.01 (t, 3H).

### Example 838

### 5-{(3R)-1-[1-(dimethylamino)propan-2-yl]-5′,6′-dihydrospiro[pyrrolidine-3,4′-pyrrolo[1,2-b]pyrazol]-2′-yl}-3-(trifluoromethyl)pyridin-2-amine (Diastereomer 1 and Diastereomer 2)

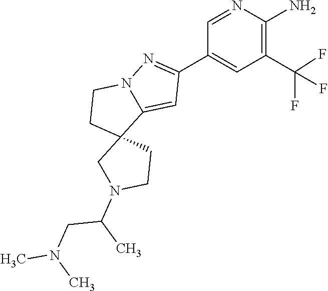 [SMILES: CC(CN(C)C)N1CC[C@@]2(CCn3nc(-c4cnc(N)c(C(F)(F)F)c4)cc32)C1]

5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (250 mg, 79% purity, 549 μmol) and 2-chloro-N,N-dimethylpropan-1-amine hydrogen chloride (1/1) (104 mg, 659 μmol) were dissolved in THF (6 mL). Potassium carbonate (303 mg, 2.20 mmol) and sodium iodide (85 mg, 549 μmol) were added and the mixture was stirred for 48 h at 70° C. The reaction mixture was allowed to cool down, diluted with water and extracted with ethyl acetate. The combined organic layers were washed with brine, dried over sodium sulfate, filtered and concentrated. The residue was purified by preparative HPLC to give 41.0 mg (95% purity, 17% yield) of the title compound.

[α]²⁰_{D}: +36.8° (c=1.00, DMSO)

LC-MS (Method 1): Rₜ=1.15 min; MS (ESIpos): m/z=410 [M+H]⁺

¹H NMR (DMSO-d₆, 400 MHz): δ (ppm) 8.58 (d, 1H), 8.01 (d, 1H), 6.52 (s, 2H), 6.42 and 6.43 (2s, 1H), 4.10 (t, 2H), 2.53-2.85 (m, 6H), 2.36-2.48 (m, 3H), 2.16 (2s, 6H), 1.93-2.08 (m, 2H), 0.94 and 0.95 (2d, 3H).

### Example 839

(3R)-2′-{6-amino-5-[(1R)-1-(1,2-thiazol-5-yl)ethoxy]pyridin-3-yl}-N-ethyl-5′,6′-dihydrospiro[pyrrolidine-3,4′-pyrrolo[1,2-b]pyrazole]-1-carboxamide

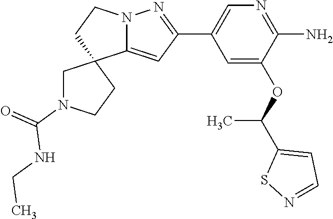 [SMILES: CCNC(=O)N1CC[C@@]2(CCn3nc(-c4cnc(N)c(O[C@H](C)c5ccns5)c4)cc32)C1]

5-(4,4,5,5-Tetramethyl-1,3,2-dioxaborolan-2-yl)-3-[(1R)-1-(1,2-thiazol-5-yl)ethoxy]pyridin-2-amine (150 mg, 62% purity, 268 μmol) was dissolved in 1,4-dioxane (1.5 mL) under argon. (3R)-1-(Ethylcarbamoyl)-5′,6′-dihydrospiro[pyrrolidine-3,4′-pyrrolo[1,2-b]pyrazol]-2′-yl trifluoromethanesulfonate (85.3 mg, 223 μmol), potassium phosphate (1.3 mL, 0.50 M, 670 μmol) and XPhos Pd G2 (8.78 mg, 11.2 μmol) were added and the mixture was stirred for 1 h at 100° C. The cold reaction mixture was diluted with dichloromethane and washed with water and brine. The organic phase was dried over a hydrophobic filter and concentrated. The crude product was purified by preparative HPLC to give 18.7 mg (98% purity, 18% yield) of the title compound.

[α]²⁰_{D}: +101.0° (c=1.00, methanol)

LC-MS (Method 1): Rₜ=0.81 min; MS (ESIpos): m/z=454 [M+H]⁺

¹H NMR (DMSO-d₆, 400 MHz): δ (ppm) 8.49 (d, 1H), 7.94 (d, 1H), 7.46 (d, 1H), 7.43 (d, 1H), 6.30 (s, 1H), 6.08-6.17 (m, 2H), 5.84 (s, 2H), 4.14 (t, 2H), 3.44-3.51 (m, 1H), 3.35-3.42 (m, 3H), 3.01-3.09 (m, 2H), 1.98-2.10 (m, 2H), 1.69 (d, 3H), 1.02 (t, 3H).

### Example 840

### 6-({(3R)-2′-[6-amino-5-(trifluoromethyl)pyridin-3-yl]-5′,6′-dihydrospiro[pyrrolidine-3,4′-pyrrolo[1,2-b]pyrazol]-1-yl}methyl)pyridin-2(1H)-one

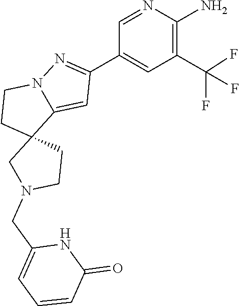 [SMILES: Nc1ncc(-c2cc3n(n2)CC[C@@]32CCN(Cc3cccc(=O)[nH]3)C2)cc1C(F)(F)F]

5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (60.0 mg, 167 μmol), 6-oxo-1,6-dihydropyridine-2-carbaldehyde (20.5 mg, 167 μmol), sodium triacetoxyborohydride (177 mg, 834 μmol) and trifluoroacetic acid (58 μL, 750 μmol) were dissolved in DMF (2.8 mL) and stirred for 12 h at 50° C. The reaction mixture was poured into a sodium bicarbonate solution and extracted with ethyl acetate. The combined organic layers were dried over sodium sulfate, filtered and concentrated. The residue was purified by preparative TLC (dichloromethane/methanol 9:1 gradient) to give 16.1 mg (95% purity, 21% yield) of the title compound.

LC-MS (Method 1): Rₜ=0.93 min; MS (ESIpos): m/z=431 [M+H]⁺

¹H-NMR (400 MHz, CHLOROFORM-d) δ [ppm]: 2.083 (0.65), 2.102 (1.02), 2.116 (1.18), 2.120 (0.99), 2.133 (1.48), 2.153 (1.08), 2.243 (1.09), 2.256 (1.32), 2.264 (1.44), 2.277 (1.86), 2.289 (1.00), 2.297 (0.96), 2.310 (0.85), 2.599 (0.66), 2.631 (1.43), 2.647 (1.54), 2.651 (1.64), 2.667 (1.34), 2.696 (1.42), 2.709 (2.55), 2.715 (1.77), 2.729 (3.58), 2.741 (1.21), 2.748 (2.46), 2.752 (1.44), 2.761 (0.92), 2.769 (1.02), 2.782 (3.22), 2.805 (4.77), 2.887 (4.29), 2.893 (2.69), 2.911 (2.85), 2.940 (1.00), 2.952 (1.18), 2.960 (1.41), 2.967 (3.16), 2.972 (1.33), 2.983 (0.93), 2.995 (0.75), 3.539 (0.46), 3.608 (10.27), 4.153 (0.69), 4.169 (0.84), 4.173 (0.85), 4.181 (1.93), 4.189 (0.84), 4.197 (2.17), 4.200 (2.36), 4.210 (1.94), 4.216 (1.96), 4.223 (2.22), 4.230 (2.02), 4.237 (0.92), 4.243 (1.86), 4.251 (0.82), 4.257 (0.80), 4.271 (0.65), 5.014 (5.18), 5.310 (5.59), 6.036 (2.75), 6.038 (2.72), 6.052 (2.87), 6.055 (2.85), 6.241 (16.00), 6.458 (2.89), 6.480 (3.04), 7.006 (0.62), 7.324 (3.83), 7.340 (3.59), 7.347 (3.40), 7.364 (3.39), 7.528 (0.65), 8.117 (3.59), 8.121 (3.58), 8.622 (3.47), 8.626 (3.34), 9.630 (0.69).

### Example 841

### (3R)-2′-{6-amino-5-[(1R)-1-(pyridin-3-yl)ethoxy]pyridin-3-yl}-N-(propan-2-yl)-5′,6′-dihydrospiro[pyrrolidine-3,4′-pyrrolo[1,2-b]pyrazole]-1-carboxamide

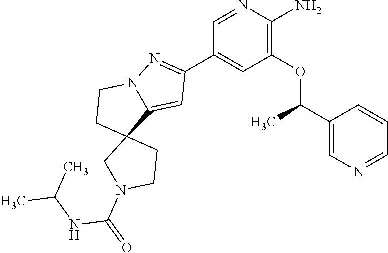 [SMILES: CC(C)NC(=O)N1CC[C@@]2(CCn3nc(-c4cnc(N)c(O[C@H](C)c5cccnc5)c4)cc32)C1]

5-[(3R)-5′,6′-Dihydrospiro[pyrrolidine-3,4′-pyrrolo[1,2-b]pyrazol]-2′-yl]-3-[(1R)-1-(pyridin-3-yl)ethoxy]pyridin-2-amine hydrogen chloride (1/1) (36.0 mg, 87.2 μmol) was suspended in dichloromethane (270 μL). N,N-Diisopropylethylamine (150 μL, 870 μmol) was added and the mixture was cooled to 0° C. and 2-isocyanatopropane (10 μL, 100 μmol) was added. The mixture was allowed to warm up to rt and stirred over night. The reaction mixture was concentrated. The residue was purified by preparative TLC (dichloromethane/methanol 9:1 gradient) to give 7.70 mg (96% purity, 18% yield) of the title compound.

LC-MS (Method 1): Rₜ=0.85 min; MS (ESIpos): m/z=462 [M+H]⁺

¹H-NMR (400 MHz, CHLOROFORM-d) δ [ppm]: 1.087 (0.67), 1.093 (3.16), 1.103 (3.91), 1.109 (3.97), 1.118 (3.06), 1.184 (2.24), 1.633 (4.40), 1.649 (4.45), 1.990 (0.44), 2.142 (0.52), 2.464 (0.44), 2.482 (0.57), 2.539 (0.63), 2.558 (0.47), 2.572 (0.42), 3.415 (1.20), 3.436 (1.60), 3.457 (0.68), 3.460 (0.66), 3.914 (1.00), 3.926 (0.66), 3.941 (0.43), 4.141 (1.10), 4.159 (1.91), 4.177 (1.02), 4.706 (1.80), 5.233 (0.66), 5.452 (0.72), 5.468 (0.72), 6.013 (4.14), 7.194 (16.00), 7.206 (1.94), 7.210 (1.48), 7.217 (0.67), 7.218 (0.66), 7.224 (0.63), 7.226 (0.63), 7.236 (0.61), 7.238 (0.62), 7.629 (0.41), 7.634 (0.70), 7.639 (0.44), 7.654 (0.65), 7.922 (2.00), 7.927 (1.98), 8.466 (0.97), 8.470 (1.01), 8.478 (0.99), 8.482 (0.95), 8.603 (1.10), 8.608 (1.09).

### Example 842

### 2′-[6-amino-5-(6,7-dihydro-5H-cyclopenta[b]pyridin-7-yloxy)pyridin-3-yl]-N-ethyl-5′,6′-dihydro-1H-spiro[pyrrolidine-3,4′-pyrrolo[1,2-b]pyrazole]-1-carboxamide (4 Isomers)

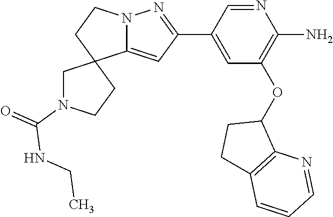 [SMILES: CCNC(=O)N1CCC2(CCn3nc(-c4cnc(N)c(OC5CCc6cccnc65)c4)cc32)C1]

3-[(6,7-Dihydro-5H-cyclopenta[b]pyridin-7-yl)oxy]-5-[5′,6′-dihydrospiro(pyrrolidine-3,4′-pyrrolo[1,2-b]pyrazol)-2′-yl]pyridin-2-amine dihydrogen chloride (1/2) (89.0 mg, 193 μmol) was suspended in dichloromethane (3.1 mL). N,N-Diisopropylethylamine (200 μL, 1.2 mmol) and isocyanatoethane (16 μL, 200 μmol) were added and stirred at rt. The reaction mixture was diluted with water and stirred for 5 min. The layers were separated and the aqueous phase was extracted with dichloromethane/methanol 9:1. The combined organic layers were concentrated. The residue was purified by flash column chromatography (silica gel, dichloromethane/methanol (0-5%) gradient) to give 52.0 mg (90% purity, 53% yield) of the title compound.

LC-MS (Method 1): Rₜ=0.85 min; MS (ESIpos): m/z=460 [M+H]⁺

¹H NMR (DMSO-d₆, 400 MHz): δ (ppm) 8.43-8.47 (m, 1H), 7.97 (d, 1H), 7.78 (dd, 1H), 7.75 (d, 1H), 7.32 (dd, 1H), 6.35 (s, 1H), 6.16 (t, 1H), 5.69-5.77 (m, 3H), 4.18 (t, 2H), 3.46-3.54 (m, 1H), 3.36-3.44 (m, 3H), 3.01-3.18 (m, 3H), 2.91 (ddd, 1H), 2.55-2.63 (m, 1H), 2.20 (qd, 1H), 2.00-2.13 (m, 2H), 1.02 (t, 3H).

### Example 843

### (3R)-2′-{6-amino-5-[1-(1,3-oxazol-4-yl)ethoxy]pyridin-3-yl}-N-isopropyl-5′,6′-dihydro-1H-spiro[pyrrolidine-3,4′-pyrrolo[1,2-b]pyrazole]-1-carboxamide (Diastereomer 1 and Diastereomer 2)

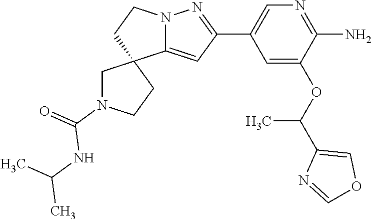 [SMILES: CC(C)NC(=O)N1CC[C@@]2(CCn3nc(-c4cnc(N)c(OC(C)c5cocn5)c4)cc32)C1]

3-{[1-(1,3-Oxazol-4-yl)ethyl]oxy}-5-(4,4,5,5-tetramethyl-1,3,2-dioxaborolan-2-yl)pyridin-2-amine (275 mg, 36% purity, 296 μmol) was dissolved in 1,4-dioxane (1.7 mL) under argon. (3R)-1-[(Propan-2-yl)carbamoyl]-5′,6′-dihydrospiro[pyrrolidine-3,4′-pyrrolo[1,2-b]pyrazol]-2′-yl trifluoromethanesulfonate (97.9 mg, 247 μmol), potassium phosphate (1.5 mL, 0.50 M, 740 μmol) and XPhos Pd G2 (19.4 mg, 24.7 μmol) were added and the mixture was stirred for 1 h at 100° C. The cold reaction mixture was diluted with dichloromethane, washed with water and brine. The organic phase was dried over a hydrophobic filter and concentrated. The residue was purified by preparative HPLC to give 4.00 mg (93% purity, 3% yield) of the title compound.

LC-MS (Method 1): Rₜ=0.82 min; MS (ESIpos): m/z=452 [M+H]⁺

1H-NMR (400 MHz, DMSO-d6): δ [ppm]=1.06 (d, 3H), 1.07 (d, 3H), 1.59 (d, 3H), 1.98-2.10 (m, 2H), 3.36-3.43 (m, 3H), 3.46-3.54 (m, 1H), 3.71-3.80 (m, 1H), 4.16 (t, 2H), 5.49 (q, 1H), 5.75 (s, 2H), 5.82 (d, 1H), 6.33 (s, 1H), 7.44 (d, 1H), 7.94 (d, 1H), 8.17 (d, 1H), 8.38 (s, 1H).

### Example 844

### 5-{(3R)-1-[(1,3-thiazol-2-yl)methyl]-5′,6′-dihydrospiro[pyrrolidine-3,4′-pyrrolo[1,2-b]pyrazol]-2′-yl}-3-(trifluoromethyl)pyridin-2-amine

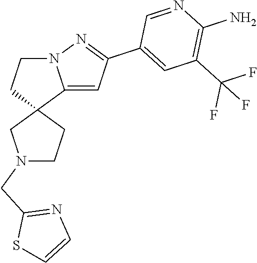 [SMILES: Nc1ncc(-c2cc3n(n2)CC[C@@]32CCN(Cc3nccs3)C2)cc1C(F)(F)F]

5-[(3R)-5′,6′-Dihydrospiro[pyrrolidine-3,4′-pyrrolo[1,2-b]pyrazol]-2′-yl]-3-(trifluoromethyl)pyridin-2-amine (100 mg, 309 μmol) and 1,3-thiazole-2-carbaldehyde (35.0 mg, 309 μmol, CAS 10200-59-6) were dissolved in methanol (1.1 mL). N,N-Diisopropylethylamine (220 μL, 1.2 mmol) and titanium(IV) isopropoxide (270 μL, 930 μmol) were added and the mixture was stirred for 16 h at 60° C. To the cold reaction mixture was added sodium cyanoborohydride (58.3 mg, 928 μmol) and stirred for 2 h at 60° C. The reaction mixture was allowed to cool down, diluted with water (1 mL) and stirred for 30 min. The suspension was diluted with methanol, filtered over celite and the filter cake was washed with methanol. The filtrate was concentrated. The residue was dissolved in dichloromethane/methanol (small amount of methanol) and washed twice with a saturated sodium bicarbonate solution. The aqueous phase was extracted with dichloromethane/methanol. The combined organic layers were dried over a hydrophobic filter and concentrated. The residue was purified by flash column chromatography (silica gel, dichloromethane/methanol (0-4%) gradient) to give 65.0 mg (97% purity, 48% yield) of the title compound.

LC-MS (Method 1): Rₜ=1.09 min; MS (ESIpos): m/z=421 [M+H]⁺

¹H-NMR (400 MHz, DMSO-d6) δ [ppm]: 1.238 (1.41), 1.253 (1.06), 1.269 (0.59), 1.988 (0.46), 2.039 (0.48), 2.055 (1.50), 2.071 (2.75), 2.084 (3.51), 2.089 (2.90), 2.104 (1.67), 2.116 (0.46), 2.121 (0.50), 2.318 (0.49), 2.323 (1.06), 2.327 (1.55), 2.332 (1.13), 2.336 (0.47), 2.518 (5.84), 2.523 (4.12), 2.562 (2.03), 2.578 (1.32), 2.592 (1.29), 2.608 (1.98), 2.611 (1.75), 2.626 (1.80), 2.641 (1.06), 2.660 (1.00), 2.665 (1.19), 2.669 (1.60), 2.673 (1.15), 2.765 (3.21), 2.788 (4.83), 2.818 (0.68), 2.841 (1.63), 2.855 (6.12), 2.878 (4.02), 2.896 (1.14), 2.911 (1.57), 2.916 (1.62), 2.931 (1.35), 2.954 (0.60), 4.002 (0.66), 4.039 (10.88), 4.043 (11.08), 4.081 (0.69), 4.090 (0.48), 4.100 (2.21), 4.105 (2.07), 4.118 (3.85), 4.124 (2.66), 4.135 (2.07), 4.140 (2.14), 4.152 (0.42), 5.759 (14.68), 6.482 (16.00), 6.525 (7.87), 7.656 (8.72), 7.664 (11.85), 7.711 (11.15), 7.719 (8.33), 8.013 (4.44), 8.018 (4.45), 8.589 (4.12), 8.594 (3.99).

### Example 845

### 6-({(3′R)-2-[6-amino-5-(trifluoromethyl)pyridin-3-yl]-6,7-dihydrospiro[pyrazolo[5,1-c][1,4]oxazine-4,3′-pyrrolidin]-1′-yl}methyl)pyridin-2(1H)-one

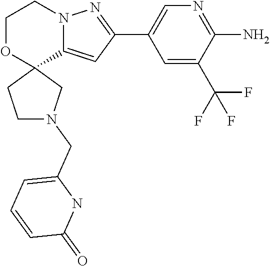

5-[(3′R)-6,7-dihydrospiro[pyrazolo[5,1-c][1,4]oxazine-4,3′-pyrrolidin]-2-yl]-3-(trifluoromethyl)pyridin-2-amine hydrogen chloride (1/1) (50.0 mg, 133 μmol), 6-oxo-1,6-dihydropyridine-2-carbaldehyde (16.4 mg, 133 μmol, CAS 358751-77-6), sodium triacetoxyborohydride (141 mg, 665 μmol) and trifluoroacetic acid (46 μL, 600 μmol) were dissolved in DMF (2.2 mL) and stirred for 12 h at 50° C. The reaction mixture was poured into a sodium bicarbonate solution and extracted with ethyl acetate. The combined organic layers were dried over sodium sulfate, filtered and concentrated. The residue was purified by preparative TLC (dichloromethane/methanol 9:1) to give 15.4 mg (98% purity, 25% yield) of the title compound.

LC-MS (Method 1): Rₜ=0.89 min; MS (ESIpos): m/z=448 [M+H]⁺

¹H-NMR (400 MHz, CHLOROFORM-d) δ [ppm]: 1.261 (0.44), 2.226 (0.78), 2.246 (1.72), 2.260 (1.43), 2.265 (1.17), 2.279 (2.61), 2.299 (1.32), 2.377 (0.95), 2.389 (1.24), 2.394 (1.33), 2.408 (1.31), 2.423 (0.82), 2.428 (0.82), 2.441 (0.68), 2.841 (0.80), 2.854 (0.96), 2.864 (1.66), 2.877 (1.76), 2.884 (1.39), 2.897 (1.16), 2.926 (1.28), 2.944 (2.73), 2.963 (1.45), 2.967 (2.03), 2.985 (0.68), 3.041 (2.65), 3.067 (7.36), 3.091 (5.29), 3.118 (1.74), 3.646 (9.23), 4.070 (0.57), 4.075 (0.56), 4.085 (0.90), 4.089 (1.58), 4.101 (2.18), 4.104 (2.93), 4.108 (1.87), 4.114 (2.32), 4.121 (4.19), 4.133 (2.59), 4.151 (0.63), 4.157 (0.85), 4.161 (1.07), 4.166 (0.43), 4.186 (3.37), 4.191 (2.83), 4.198 (4.44), 4.202 (3.95), 4.210 (1.86), 4.216 (1.82), 5.073 (6.00), 5.310 (12.49), 6.043 (3.07), 6.045 (3.17), 6.060 (3.23), 6.062 (3.14), 6.307 (16.00), 6.455 (3.35), 6.476 (3.54), 7.006 (0.52), 7.321 (4.17), 7.338 (4.14), 7.344 (4.01), 7.361 (3.60), 7.528 (0.55), 8.104 (4.16), 8.108 (4.17), 8.626 (4.13), 8.629 (4.02), 9.838 (0.78).

### Example 846

### (3S)-2′-[6-amino-5-(trifluoromethyl)pyridin-3-yl]-N-[cyclopropyl(phenyl)methyl]-5′,6′-dihydrospiro[pyrrolidine-3,4′-pyrrolo[1,2-b]pyrazole]-1-carboxamide (Diastereomer 1)

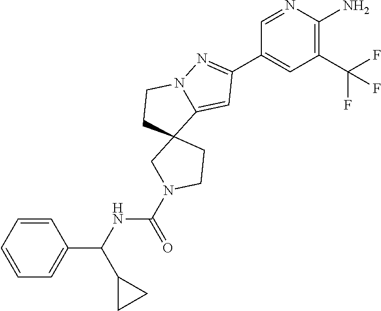 [SMILES: Nc1ncc(-c2cc3n(n2)CC[C@]32CCN(C(=O)NC(c3ccccc3)C3CC3)C2)cc1C(F)(F)F]

1-Cyclopropyl-1-phenylmethanamine (147 mg, 1 mmol) was dissolved in DMF (15 mL). CDI (162 mg, 1 mmol) and N,N-diisopropylethylamine (581 μL, 3.3 mmol) were added and stirred for 1 h at rt. 5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (300 mg, 834 μmol) was added and the mixture was stirred for 2 h at rt. The reaction mixture was concentrated and diluted with water. The aqueous phase was extracted with dichloromethane. The combined organic layers were concentrated. The residue was purified by column chromatography (silica gel, ethyl acetate/methanol (0-10%) gradient) to give 342 mg of the title compound as a mixture of diastereomer.

The mixture was separated into diastereomers by preparative chiral HPLC to give 132 mg (95% purity, 40% yield) of the title compound (diastereomer 1 Rₜ=8.4-11.0 min).

Preparative Chiral HPLC Method:

Instrument: PrepCon Labomatic HPLC-2; Column: YMC Amylose SA 10μ, 250×50; eluent A: methyl tert-butyl ether+0.1 vol % diethylamine; eluent B: acetonitrile+0.1 vol % diethylamine; isocratic: 50% B; flow: 100 mL/min; temperature: 25° C.; UV: 254 nm

Analytical Chiral HPLC Method:

Instrument: Thermo Fisher UltiMate 3000; Column: YMC Cellulose SA 3μ, 100×4.6; eluent A: methyl tert-butyl ether+0.1 vol % diethylamine; eluent B: acetonitrile; isocratic: 50% B; flow: 1.4 mL/min; temperature: 25° C.; UV: 254 nm

Analytical chiral HPLC: Rₜ=2.38 min

[α]²⁰_{D}: −87.7° (c=1.00, DMSO)

LC-MS (Method 1): Rₜ=1.12 min; MS (ESIpos): m/z=497 [M+H]⁺

¹H NMR (DMSO-d₆, 400 MHz): δ (ppm) 8.57 (d, 1H), 8.00 (d, 1H), 7.39-7.44 (m, 2H), 7.30 (t, 2H), 7.17-7.23 (m, 1H), 6.65 (d, 1H), 6.55 (s, 2H), 6.44 (s, 1H), 4.19 (t, 2H), 4.06 (t, 1H), 3.54-3.62 (m, 1H), 3.40-3.50 (m, 3H), 2.52-2.57 (m, 2H), 2.02-2.13 (m, 2H), 1.14-1.25 (m, 1H), 0.42-0.52 (m, 2H), 0.27-0.39 (m, 2H).

### Example 847

### (3S)-2′-[6-amino-5-(trifluoromethyl)pyridin-3-yl]-N-[cyclopropyl(phenyl)methyl]-5′,6′-dihydrospiro[pyrrolidine-3,4′-pyrrolo[1,2-b]pyrazole]-1-carboxamide (Diastereomer 2)

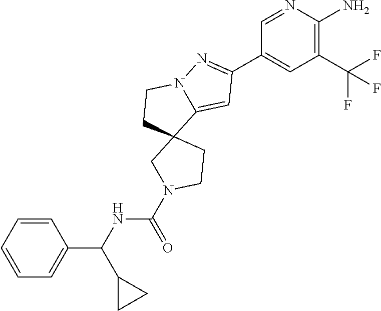 [SMILES: Nc1ncc(-c2cc3n(n2)CC[C@]32CCN(C(=O)NC(c3ccccc3)C3CC3)C2)cc1C(F)(F)F]

For the preparation of the diastereomeric mixture of the title compound see Example 846. The diastereomers were separated by preparative chiral HPLC (method see Example 846) to give 135 mg (95% purity, 41% yield) of the title compound (diastereomer 2 Rₜ=14.4-19.2 min).

Analytical chiral HPLC (method see Example 846): Rₜ=4.25 min.

[α]²⁰_{D}: −26.7° (c=1.00, DMSO)

¹H NMR (DMSO-d₆, 400 MHz): δ (ppm) 8.58 (d, 1H), 8.00 (d, 1H), 7.39 (d, 2H), 7.27 (t, 2H), 7.15-7.21 (m, 1H), 6.65 (d, 1H), 6.55 (s, 2H), 6.43 (s, 1H), 4.19 (t, 2H), 4.06 (s, 1H), 3.52-3.59 (m, 1H), 3.41-3.51 (m, 3H), 2.52-2.57 (m, 2H), 2.01-2.12 (m, 2H), 1.15-1.26 (m, 1H), 0.42-0.54 (m, 2H), 0.28-0.40 (m, 2H).

LC-MS (Method 1): Rₜ=1.12 min; MS (ESIpos): m/z=497 [M+H]⁺

### Example 848

### (3R)-2′-[6-amino-5-(6,7-dihydro-5H-cyclopenta[b]pyridin-7-yloxy)pyridin-3-yl]-N-ethyl-5′,6′-dihydro-1H-spiro[pyrrolidine-3,4′-pyrrolo[1,2-b]pyrazole]-1-carboxamide (Diastereomer 1)

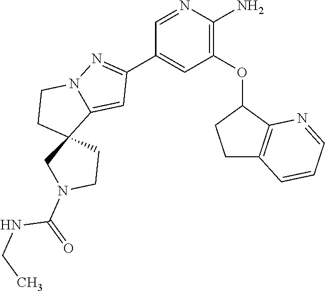 [SMILES: CCNC(=O)N1CC[C@@]2(CCn3nc(-c4cnc(N)c(OC5CCc6cccnc65)c4)cc32)C1]

(3R)-1-(Ethylcarbamoyl)-5′,6′-dihydrospiro[pyrrolidine-3,4′-pyrrolo[1,2-b]pyrazol]-2′-yl trifluoromethanesulfonate (120 mg, 313 μmol), {6-amino-5-[(6,7-dihydro-5H-cyclopenta[b]pyridin-7-yl)oxy]pyridin-3-yl}boronic acid (enantiomer 1) (212 mg, 40% purity, 313 μmol) and potassium phosphate (1.9 mL, 0.50 M, 940 μmol) were dissolved in degassed 1,4-dioxane (5.1 mL) under argon. XPhos Pd G2 (12.3 mg, 15.6 μmol) was added and the mixture was stirred for 2 h at 100° C. The reaction mixture was allowed to cool down, diluted with ethyl acetate and filtered. The filtrate was washed with water and brine. The organic phase was dried over sodium sulfate, filtered and concentrated. The residue was purified by preparative HPLC followed by preparative TLC (dichloromethane/methanol 9:1 gradient) to give 22.2 mg (98% purity, 15% yield) of the title compound.

[α]²⁰_{D}: +140.2° (c=1.00, methanol)

LC-MS (Method 1): Rₜ=0.86 min; MS (ESIpos): m/z=461 [M+H]⁺

¹H-NMR (400 MHz, CHLOROFORM-d) δ [ppm]: 1.149 (5.84), 1.168 (13.30), 1.185 (6.05), 1.839 (1.44), 2.018 (0.96), 2.055 (0.43), 2.069 (0.65), 2.086 (1.01), 2.100 (0.84), 2.119 (0.60), 2.219 (0.60), 2.238 (1.16), 2.250 (0.60), 2.256 (0.76), 2.269 (0.82), 2.283 (0.54), 2.287 (0.53), 2.296 (0.53), 2.305 (0.59), 2.318 (0.99), 2.329 (1.02), 2.342 (0.69), 2.350 (0.66), 2.353 (0.61), 2.364 (0.51), 2.523 (0.42), 2.540 (0.96), 2.556 (1.12), 2.573 (1.56), 2.590 (0.84), 2.614 (0.88), 2.631 (1.80), 2.641 (0.71), 2.649 (1.65), 2.659 (0.77), 2.663 (1.64), 2.676 (0.62), 2.680 (1.28), 2.694 (0.52), 2.697 (0.53), 2.701 (0.50), 2.715 (0.45), 2.898 (0.49), 2.912 (0.48), 2.919 (0.43), 2.939 (0.72), 2.952 (0.70), 2.960 (0.64), 2.974 (0.55), 3.122 (0.61), 3.136 (0.65), 3.144 (0.63), 3.158 (0.63), 3.176 (0.44), 3.185 (0.43), 3.279 (0.85), 3.293 (1.10), 3.297 (2.63), 3.311 (2.91), 3.315 (2.75), 3.328 (2.62), 3.333 (1.03), 3.347 (0.84), 3.484 (1.34), 3.504 (1.19), 3.528 (3.19), 3.553 (1.33), 3.568 (3.32), 3.593 (1.45), 3.616 (0.58), 3.630 (0.71), 3.635 (0.83), 3.649 (0.71), 3.660 (0.49), 4.176 (0.69), 4.190 (1.24), 4.202 (0.65), 4.249 (2.77), 4.267 (5.08), 4.285 (2.59), 4.995 (4.14), 5.297 (12.20), 5.660 (1.31), 5.671 (1.40), 5.677 (1.42), 5.688 (1.27), 6.207 (9.43), 7.214 (1.70), 7.226 (1.77), 7.233 (1.89), 7.245 (1.92), 7.260 (16.00), 7.628 (1.63), 7.632 (1.64), 7.647 (1.51), 7.651 (1.48), 7.698 (3.44), 7.703 (3.47), 8.085 (4.45), 8.090 (4.39), 8.518 (1.63), 8.520 (1.62), 8.522 (1.65), 8.530 (1.64), 8.532 (1.58), 8.534 (1.56).

### Example 849

### 2′-{6-amino-5-[(1R)-1-(pyridin-2-yl)ethoxy]pyridin-3-yl}-N-ethyl-5′,6′-dihydrospiro[azetidine-3,4′-pyrrolo[1,2-b]pyrazole]-1-carboxamide

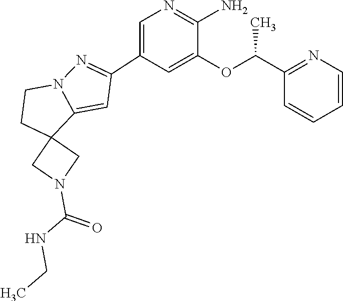 [SMILES: CCNC(=O)N1CC2(CCn3nc(-c4cnc(N)c(O[C@H](C)c5ccccn5)c4)cc32)C1]

3-[(1R)-1-(Pyridin-2-yl)ethoxy]-5-(4,4,5,5-tetramethyl-1,3,2-dioxaborolan-2-yl)pyridin-2-amine (109 mg, 319 μmol), 1-(ethylcarbamoyl)-5′,6′-dihydrospiro[azetidine-3,4′-pyrrolo[1,2-b]pyrazol]-2′-yl trifluoromethanesulfonate (129 mg, 351 μmol) and potassium phosphate (1.9 mL, 0.50 M, 960 μmol) were dissolved in degassed 1,4-dioxane. XPhos Pd G2 (12.6 mg, 16.0 μmol) was added and the mixture was stirred for 2 h at 100° C. The reaction mixture was allowed to cool down, diluted with water and extracted with ethyl acetate. The combined organic layers were washed with brine, dried over sodium sulfate, filtered and concentrated. The residue was purified by preparative HPLC to give 34.0 mg (95% purity, 23% yield) of the title compound.

LC-MS (Method 1): Rₜ=0.80 min; MS (ESIpos): m/z=435 [M+H]⁺

¹H NMR (DMSO-d₆, 400 MHz): δ (ppm) 8.53-8.56 (m, 1H), 7.90 (d, 1H), 7.79 (td, 1H), 7.51 (d, 1H), 7.29 (ddd, 1H), 7.24 (d, 1H), 6.48 (s, 1H), 6.44 (t, 1H), 5.89 (s, 2H), 5.53 (q, 1H), 4.03-4.10 (m, 2H), 3.91-3.98 (m, 4H), 2.98-3.06 (m, 2H), 2.79 (t, 2H), 1.62 (d, 3H), 1.01 (t, 3H).

### Example 850

### 1-{(3R)-2′-[6-amino-5-(trifluoromethyl)pyridin-3-yl]-5′,6′-dihydrospiro[pyrrolidine-3,4′-pyrrolo[1,2-b]pyrazol]-1-yl}-2-(methylamino)ethan-1-one

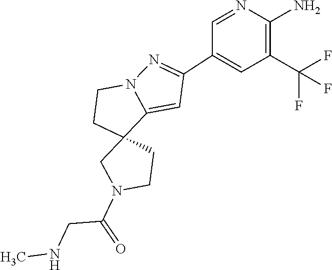 [SMILES: CNCC(=O)N1CC[C@@]2(CCn3nc(-c4cnc(N)c(C(F)(F)F)c4)cc32)C1]

Tert-butyl (2-{(3R)-2′-[6-amino-5-(trifluoromethyl)pyridin-3-yl]-5′,6′-dihydrospiro[pyrrolidine-3,4′-pyrrolo[1,2-b]pyrazol]-1-yl}-2-oxoethyl)methylcarbamate (43.5 mg, 88.0 μmol, Intermediate 550) was dissolved in dichloromethane. Hydrochloric acid in 1,4-dioxane (220 μL, 4.0 M, 880 μmol) was added and the mixture was stirred for 1 h at room temperature. The reaction mixture was diluted with water, basified with aqueous sodium hydroxide (30%) and extracted with dichloromethane. The combined organic layers were dried over a hydrophobic filter and concentrated to give 34.0 mg (99% purity, 97% yield) of the title compound.

LC-MS (Method 1): Rₜ=0.82 min; MS (ESIpos): m/z=395 [M+H]⁺

¹H-NMR (400 MHz, DMSO-d6) δ [ppm]: 1.844 (0.49), 2.024 (0.53), 2.040 (0.82), 2.052 (1.08), 2.069 (0.48), 2.110 (0.85), 2.127 (1.83), 2.144 (0.90), 2.264 (10.89), 2.307 (11.71), 2.322 (0.52), 2.327 (0.60), 2.332 (0.44), 2.522 (2.11), 2.565 (2.36), 2.580 (1.13), 2.596 (0.40), 2.665 (0.41), 2.669 (0.56), 2.673 (0.42), 3.143 (0.44), 3.183 (1.79), 3.205 (1.80), 3.245 (0.52), 3.250 (0.70), 3.291 (1.74), 3.330 (16.00), 3.368 (0.66), 3.484 (0.52), 3.509 (3.03), 3.540 (1.00), 3.588 (0.67), 3.606 (1.87), 3.632 (1.23), 3.658 (0.80), 3.675 (0.49), 3.684 (0.45), 4.166 (0.77), 4.176 (1.73), 4.183 (1.67), 4.194 (3.02), 4.211 (1.42), 6.504 (4.09), 6.521 (4.46), 6.542 (5.22), 8.008 (1.57), 8.013 (1.74), 8.019 (1.60), 8.025 (1.52), 8.591 (2.81), 8.595 (2.77).

### Example 851

### (2R)-1-{(3R)-2′-[6-amino-5-(trifluoromethyl)pyridin-3-yl]-5′,6′-dihydrospiro[pyrrolidine-3,4′-pyrrolo[1,2-b]pyrazol]-1-yl}-2-(methylamino)propan-1-one

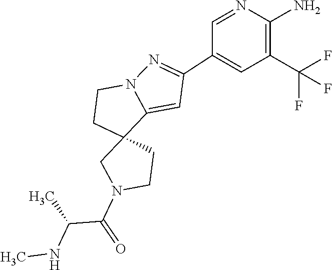 [SMILES: CN[C@H](C)C(=O)N1CC[C@@]2(CCn3nc(-c4cnc(N)c(C(F)(F)F)c4)cc32)C1]

Tert-butyl [(2R)-1-{(3R)-2′-[6-amino-5-(trifluoromethyl)pyridin-3-yl]-5′,6′-dihydrospiro[pyrrolidine-3,4′-pyrrolo[1,2-b]pyrazol]-1-yl}-1-oxopropan-2-yl]methylcarbamate (29.3 mg, 57.6 μmol, Intermediate 551) was dissolved in dichloromethane. Hydrochloric acid in 1,4-dioxane (140 μL, 4.0 M, 580 μmol) was added and the mixture was stirred for 1 h at room temperature. The reaction mixture was diluted with water, basified with aqueous sodium hydroxide (30%) and extracted with dichloromethane. The combined organic layers were dried over a hydrophobic filter and concentrated to give 23.0 mg (95% purity, 93% yield) of the title compound.

LC-MS (Method 1): Rₜ=0.84 min; MS (ESIpos): m/z=409 [M+H]⁺

¹H-NMR (400 MHz, DMSO-d6) δ [ppm]: 1.056 (1.25), 1.072 (1.30), 1.119 (1.15), 1.135 (1.19), 2.133 (0.71), 2.140 (0.75), 2.157 (0.47), 2.176 (0.73), 2.189 (0.53), 2.518 (1.80), 2.523 (1.26), 2.560 (0.43), 3.479 (0.44), 3.566 (16.00), 3.578 (0.41), 3.647 (0.49), 3.714 (0.51), 4.186 (0.54), 4.199 (0.41), 4.205 (0.62), 6.483 (1.31), 6.489 (1.49), 6.545 (1.32), 8.005 (0.77), 8.011 (0.78), 8.575 (0.41), 8.582 (0.49).

### Example 852

### (5R)-5-({(3R)-2′-[6-amino-5-(trifluoromethyl)pyridin-3-yl]-5′,6′-dihydrospiro[pyrrolidine-3,4′-pyrrolo[1,2-b]pyrazol]-1-yl}methyl)pyrrolidin-2-one

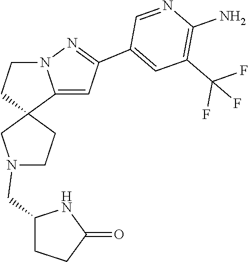 [SMILES: Nc1ncc(-c2cc3n(n2)CC[C@@]32CCN(C[C@H]3CCC(=O)N3)C2)cc1C(F)(F)F]

5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (250 mg, 695 μmol), (5R)-5-(bromomethyl)pyrrolidin-2-one (136 mg, 764 μmol, CAS 98612-60-3) and cesium carbonate (249 mg, 764 μmol) were suspended in DMF (10 mL). The reaction mixture was stirred over night at room temperature, for 1 h at 50° C. and for 1 h at 80° C. The suspension was diluted with ethyl acetate and filtered. The organic phase was washed with water and brine, dried and concentrated. The residue was purified by column chromatography (silica gel, dichloromethane/methanol (0-9%) gradient) to give 57.0 mg (99% purity, 19% yield) of the title compound.

LC-MS (Method 1): Rₜ=0.92 min; MS (ESIpos): m/z=421 [M+H]⁺

¹H-NMR (400 MHz, DMSO-d6) δ [ppm]: 0.852 (0.48), 1.154 (1.22), 1.172 (2.41), 1.190 (1.16), 1.232 (1.56), 1.712 (1.52), 1.722 (1.14), 1.736 (1.39), 1.749 (0.75), 1.773 (0.43), 1.968 (0.66), 1.980 (1.54), 1.988 (5.26), 1.998 (3.07), 2.014 (2.64), 2.033 (1.56), 2.051 (0.55), 2.069 (1.13), 2.085 (1.80), 2.102 (1.21), 2.112 (6.78), 2.121 (2.01), 2.130 (3.07), 2.135 (2.62), 2.139 (3.17), 2.155 (0.61), 2.164 (1.72), 2.188 (0.75), 2.318 (1.16), 2.323 (2.49), 2.327 (3.48), 2.331 (2.47), 2.336 (1.12), 2.397 (1.58), 2.413 (1.73), 2.427 (2.51), 2.443 (2.66), 2.518 (14.67), 2.523 (10.09), 2.561 (1.85), 2.574 (1.31), 2.590 (1.99), 2.608 (1.86), 2.621 (4.37), 2.643 (5.48), 2.665 (3.96), 2.669 (4.51), 2.673 (2.76), 2.678 (1.41), 2.684 (0.93), 2.765 (3.90), 2.787 (3.34), 2.806 (1.40), 2.812 (1.63), 2.828 (1.52), 2.848 (0.64), 3.620 (1.20), 3.634 (1.75), 3.650 (1.24), 4.018 (0.95), 4.035 (0.96), 4.091 (2.40), 4.110 (4.57), 4.129 (2.33), 5.760 (6.66), 6.487 (16.00), 6.521 (8.00), 7.634 (4.28), 8.007 (4.55), 8.012 (4.55), 8.585 (4.21), 8.590 (4.16).

### Example 853

### 5-{(3R)-1-[(1,3,4-thiadiazol-2-yl)methyl]-5′,6′-dihydrospiro[pyrrolidine-3,4′-pyrrolo[1,2-b]pyrazol]-2′-yl}-3-(trifluoromethyl)pyridin-2-amine

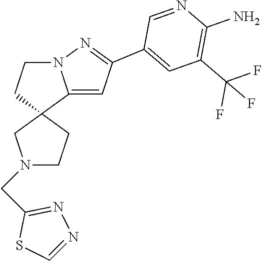 [SMILES: Nc1ncc(-c2cc3n(n2)CC[C@@]32CCN(Cc3nncs3)C2)cc1C(F)(F)F]

5-[(3R)-5′,6′-Dihydrospiro[pyrrolidine-3,4′-pyrrolo[1,2-b]pyrazol]-2′-yl]-3-(trifluoromethyl)pyridin-2-amine (100 mg, 309 μmol) and 1,3,4-thiadiazole-2-carbaldehyde (35.3 mg, 309 μmol, CAS 144876-24-4) were dissolved in methanol (1.1 mL). N,N-Diisopropylethylamine (220 μL, 1.2 mmol), titanium(IV) isopropoxide (270 μL, 930 μmol) were added and stirred for 16 h at 60° C. To the cold reaction mixture was added sodium cyanoborohydride (58.3 mg, 928 μmol) and the mixture was stirred for 2 h at 60° C. The reaction mixture was allowed to cool down, diluted with water (1 mL) and stirred for 30 min. The suspension was diluted with methanol and filtered over celite. The filtrate was concentrated. The residue was diluted with dichloromethane/methanol (small amount of methanol) and washed with a saturated sodium bicarbonate solution. The aqueous phase was extracted with dichloromethane/methanol. The combined organic layers were dried over a hydrophobic filter and concentrated. The crude product was purified by column chromatography (silica gel, dichloromethane/methanol (0-4%) gradient). The combined fractions were washed with a saturated sodium bicarbonate solution. The organic phase was dried over a hydrophobic filter and concentrated to give 18.0 mg (100% purity, 14% yield) of the title compound.

LC-MS (Method 1): Rₜ=0.97 min; MS (ESIpos): m/z=422 [M+H]⁺

¹H-NMR (400 MHz, DMSO-d6) δ [ppm]: 0.852 (0.49), 1.232 (2.39), 2.039 (0.40), 2.052 (1.40), 2.071 (2.25), 2.080 (2.12), 2.084 (2.24), 2.099 (1.56), 2.115 (0.41), 2.331 (1.73), 2.336 (0.76), 2.518 (9.61), 2.523 (6.77), 2.570 (1.65), 2.573 (1.63), 2.589 (2.04), 2.607 (1.70), 2.622 (0.95), 2.639 (0.58), 2.673 (1.75), 2.678 (0.81), 2.754 (2.95), 2.776 (4.69), 2.820 (0.66), 2.832 (5.04), 2.842 (1.69), 2.855 (3.79), 2.872 (1.40), 2.878 (1.34), 2.888 (1.79), 2.891 (1.64), 2.907 (1.30), 2.930 (0.48), 4.096 (2.10), 4.101 (1.95), 4.115 (3.90), 4.131 (1.99), 4.135 (2.05), 4.156 (0.65), 4.193 (10.25), 4.196 (10.48), 4.234 (0.57), 5.760 (7.06), 6.475 (16.00), 6.528 (7.29), 8.013 (4.08), 8.018 (4.16), 8.591 (3.77), 8.595 (3.73), 9.566 (15.56).

### Example 854

### (5S)-5-({(3R)-2′-[6-amino-5-(trifluoromethyl)pyridin-3-yl]-5′,6′-dihydrospiro[pyrrolidine-3,4′-pyrrolo[1,2-b]pyrazol]-1-yl}methyl)pyrrolidin-2-one

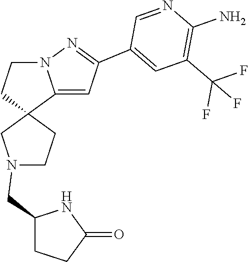 [SMILES: Nc1ncc(-c2cc3n(n2)CC[C@@]32CCN(C[C@@H]3CCC(=O)N3)C2)cc1C(F)(F)F]

5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (250 mg, 695 μmol), (5S)-5-(bromomethyl)pyrrolidin-2-one (136 mg, 764 μmol, CAS 72479-05-1) and cesium carbonate (249 mg, 764 μmol) were suspended in DMF (10 mL). The reaction mixture was stirred over night at room temperature, for 1 h at 50° C. and for 1 h at 80° C. The suspension was diluted with ethyl acetate and filtered. The organic phase was washed with water and brine, dried and concentrated. The residue was purified by column chromatography (silica gel, dichloromethane/methanol (0-9%) gradient) to give 26.0 mg (98% purity, 9% yield) of the title compound.

LC-MS (Method 1): Rₜ=0.92 min; MS (ESIpos): m/z=421 [M+H]⁺

¹H-NMR (400 MHz, DMSO-d6) δ [ppm]: 0.852 (0.56), 1.154 (0.64), 1.172 (1.31), 1.190 (0.67), 1.232 (1.89), 1.705 (1.33), 1.722 (1.01), 1.735 (1.07), 1.759 (0.42), 1.944 (0.45), 1.964 (0.79), 1.975 (1.38), 1.988 (3.24), 1.992 (1.80), 2.010 (1.93), 2.024 (1.64), 2.030 (1.61), 2.044 (1.54), 2.061 (0.99), 2.067 (1.31), 2.081 (2.59), 2.111 (3.92), 2.122 (1.69), 2.130 (2.31), 2.137 (1.81), 2.144 (2.39), 2.169 (2.08), 2.193 (0.86), 2.323 (2.23), 2.327 (3.08), 2.331 (2.21), 2.373 (1.53), 2.388 (1.69), 2.402 (2.22), 2.418 (2.19), 2.449 (1.45), 2.518 (16.00), 2.523 (9.42), 2.565 (1.72), 2.584 (3.17), 2.606 (3.79), 2.619 (1.17), 2.632 (1.47), 2.638 (1.41), 2.651 (1.85), 2.665 (3.94), 2.669 (4.20), 2.673 (2.62), 2.685 (2.16), 2.703 (1.68), 2.723 (0.87), 2.758 (3.98), 2.780 (3.40), 2.808 (0.56), 3.619 (1.02), 3.633 (1.61), 3.649 (1.21), 4.018 (0.50), 4.035 (0.47), 4.053 (0.54), 4.080 (1.69), 4.096 (3.10), 4.108 (2.30), 4.114 (2.98), 4.127 (1.70), 4.140 (0.54), 5.760 (8.46), 6.494 (15.33), 6.521 (8.03), 7.654 (4.26), 8.006 (4.62), 8.012 (4.61), 8.589 (4.35), 8.592 (4.25).

### Example 855

### 5-{(3′R)-1′-[1-(6-methoxypyridin-2-yl)ethyl]-6,7-dihydrospiro[pyrazolo[5,1-c][1,4]oxazine-4,3′-pyrrolidin]-2-yl}-3-(trifluoromethyl)pyridin-2-amine (Diastereomer 1 and Diastereomer 2)

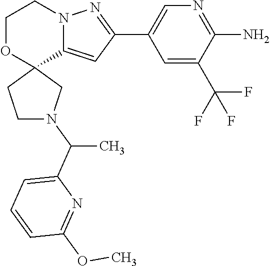 [SMILES: COc1cccc(C(C)N2CC[C@]3(C2)OCCn2nc(-c4cnc(N)c(C(F)(F)F)c4)cc23)n1]

5-[(3′R)-6,7-Dihydrospiro[pyrazolo[5,1-c][1,4]oxazine-4,3′-pyrrolidin]-2-yl]-3-(trifluoromethyl)pyridin-2-amine hydrogen chloride (1/1) (250 mg, 665 μmol) and 1-(6-methoxypyridin-2-yl)ethan-1-one (151 mg, 998 μmol, CAS 21190-93-2) were dissolved in methanol (2.5 mL). N,N-Diisopropylethylamine (580 μL, 3.3 mmol), titanium(IV) isopropoxide (590 μL, 2.0 mmol) were added and stirred for 16 h at 60° C. To the cold reaction mixture was added sodium cyanoborohydride (125 mg, 2.00 mmol) and the mixture was stirred for 2 h at 60° C. The reaction mixture was allowed to cool down, diluted with water and stirred for 30 min. The suspension was diluted with methanol and filtered over celite. The filtrate was concentrated. The residue was diluted with dichloromethane/methanol (small amount of methanol) and washed with brine. The aqueous phase was extracted with dichloromethane/methanol. The combined organic layers were dried over sodium sulfate, filtered and concentrated. The crude product was purified by column chromatography (silica gel, dichloromethane/methanol (0-5%) gradient) followed by preparative HPLC (basic HT) to give 29.2 mg (98% purity, 9% yield) of the title compound.

LC-MS (Method 1): Rₜ=1.24 min; MS (ESIpos): m/z=476 [M+H]⁺

¹H-NMR (400 MHz, CHLOROFORM-d) δ [ppm]: 1.453 (4.99), 1.459 (4.59), 1.470 (5.13), 1.476 (4.41), 2.183 (5.10), 2.210 (0.45), 2.224 (0.63), 2.227 (0.60), 2.237 (0.53), 2.243 (0.81), 2.261 (1.20), 2.281 (0.61), 2.297 (0.54), 2.303 (0.54), 2.315 (0.55), 2.348 (0.43), 2.366 (0.58), 2.382 (0.47), 2.710 (0.52), 2.725 (0.52), 2.845 (0.67), 2.867 (0.83), 2.896 (1.11), 2.923 (1.35), 2.941 (1.20), 2.967 (1.71), 2.976 (0.43), 2.989 (0.45), 2.996 (0.52), 3.009 (0.49), 3.071 (1.52), 3.096 (1.92), 3.121 (1.29), 3.138 (0.44), 3.556 (1.05), 3.570 (1.31), 3.573 (1.22), 3.586 (1.15), 3.927 (14.08), 3.931 (16.00), 4.076 (0.75), 4.079 (0.77), 4.089 (1.82), 4.093 (1.97), 4.097 (1.81), 4.101 (2.27), 4.108 (1.65), 4.161 (1.62), 4.165 (2.08), 4.175 (2.34), 4.178 (2.98), 4.190 (1.20), 5.005 (3.79), 6.290 (4.20), 6.325 (5.03), 6.589 (1.59), 6.604 (1.55), 6.608 (1.70), 6.625 (1.40), 6.981 (1.45), 7.000 (2.65), 7.007 (0.47), 7.019 (1.30), 7.516 (1.21), 7.529 (0.44), 7.534 (1.38), 7.536 (1.47), 7.543 (1.15), 7.554 (1.14), 7.561 (1.22), 7.563 (1.29), 7.582 (0.94), 8.112 (2.57), 8.609 (2.52).

### Example 856

### 5-{(3R)-1-[cyclopropyl(6-methoxypyridin-2-yl)methyl]-5′,6′-dihydrospiro[pyrrolidine-3,4′-pyrrolo[1,2-b]pyrazol]-2′-yl}-3-(trifluoromethyl)pyridin-2-amine (Diastereomer 1 and Diastereomer 2)

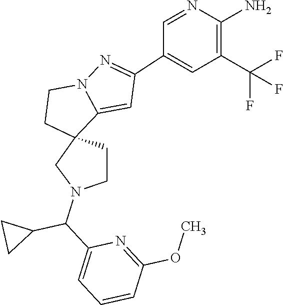 [SMILES: COc1cccc(C(C2CC2)N2CC[C@@]3(CCn4nc(-c5cnc(N)c(C(F)(F)F)c5)cc43)C2)n1]

5-[(3R)-5′,6′-Dihydrospiro[pyrrolidine-3,4′-pyrrolo[1,2-b]pyrazol]-2′-yl]-3-(trifluoromethyl)pyridin-2-amine (70.0 mg, 217 μmol) and cyclopropyl(6-methoxypyridin-2-yl)methanone (82.2 mg, 70% purity, 325 μmol, Intermediate 554) were dissolved in methanol (780 μL). N,N-Diisopropylethylamine (150 μL, 870 μmol), titanium(IV) isopropoxide (190 μL, 650 μmol) were added and stirred for 16 h at 60° C. To the cold reaction mixture was added sodium cyanoborohydride (40.8 mg, 650 μmol) and the mixture was stirred for 4 h at 60° C. The reaction mixture was allowed to cool down, diluted with water and stirred for 30 min. The suspension was diluted with methanol and filtered over celite. The filtrate was concentrated. The residue was dissolved with dichloromethane/methanol (small amount of methanol) and washed with brine. The aqueous phase was extracted with dichloromethane/methanol. The combined organic layers were dried over sodium sulfate, filtered and concentrated. The crude product was purified by column chromatography (silica gel, dichloromethane/methanol (0-5%) gradient) to give 18.4 mg (95% purity, 17% yield) of the title compound.

LC-MS (Method 1): Rₜ=1.39 min; MS (ESIpos): m/z=486 [M+H]⁺

¹H-NMR (400 MHz, CHLOROFORM-d) δ [ppm]: 0.309 (0.88), 0.320 (1.45), 0.333 (1.50), 0.342 (1.87), 0.355 (1.88), 0.366 (0.93), 0.376 (0.85), 0.388 (0.46), 0.414 (0.82), 0.426 (1.12), 0.438 (1.27), 0.450 (1.09), 0.462 (0.75), 0.474 (0.41), 0.658 (0.71), 0.672 (0.99), 0.690 (0.91), 1.128 (0.47), 1.139 (0.79), 1.150 (0.82), 1.161 (1.07), 1.172 (0.77), 1.183 (0.75), 1.195 (0.40), 1.262 (0.99), 1.997 (0.49), 2.010 (0.77), 2.025 (0.68), 2.029 (0.65), 2.044 (0.62), 2.056 (0.57), 2.072 (0.46), 2.090 (0.70), 2.110 (0.43), 2.163 (0.70), 2.171 (0.64), 2.179 (1.09), 2.192 (0.97), 2.211 (0.78), 2.224 (0.42), 2.232 (0.45), 2.460 (1.46), 2.469 (1.86), 2.484 (1.47), 2.493 (1.74), 2.539 (0.40), 2.558 (0.62), 2.571 (1.05), 2.586 (1.14), 2.603 (0.83), 2.662 (0.51), 2.675 (0.65), 2.681 (0.69), 2.693 (1.39), 2.710 (2.27), 2.725 (1.81), 2.733 (2.34), 2.759 (2.55), 2.783 (2.25), 2.840 (1.47), 2.864 (1.08), 2.918 (0.51), 2.932 (0.58), 2.940 (0.85), 2.954 (0.80), 2.977 (2.11), 3.000 (1.54), 3.138 (0.58), 3.150 (0.57), 3.914 (13.80), 3.923 (16.00), 4.119 (0.61), 4.140 (0.94), 4.155 (0.85), 4.167 (1.41), 4.183 (2.40), 4.203 (2.26), 4.218 (1.13), 4.230 (0.57), 4.989 (4.23), 5.310 (4.11), 6.203 (3.77), 6.244 (4.77), 6.599 (1.93), 6.610 (1.58), 6.619 (2.09), 6.630 (1.55), 7.013 (1.52), 7.029 (2.72), 7.045 (2.03), 7.529 (0.41), 7.535 (1.33), 7.554 (2.84), 7.574 (2.60), 7.592 (0.92), 8.110 (1.80), 8.114 (1.86), 8.126 (2.20), 8.131 (2.14), 8.608 (1.77), 8.625 (2.12).

### Example 857

### 5-{(3′R)-1′-[cyclopropyl(6-methoxypyridin-2-yl)methyl]-6,7-dihydrospiro[pyrazolo[5,1-c][1,4]oxazine-4,3′-pyrrolidin]-2-yl}-3-(trifluoromethyl)pyridin-2-amine (Diastereomer 1 and Diastereomer 2)

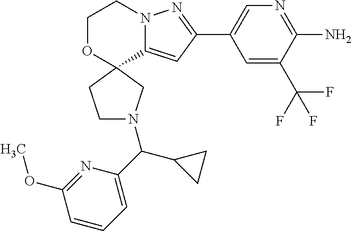 [SMILES: COc1cccc(C(C2CC2)N2CC[C@]3(C2)OCCn2nc(-c4cnc(N)c(C(F)(F)F)c4)cc23)n1]

The compound was prepared similarly to Example 856 using 5-[(3′R)-6,7-dihydrospiro[pyrazolo[5,1-c][1,4]oxazine-4,3′-pyrrolidin]-2-yl]-3-(trifluoromethyl)pyridin-2-amine (71.5 mg, 211 μmol, Intermediate 537) and cyclopropyl(6-methoxypyridin-2-yl)methanone (80.0 mg, 70% purity, 316 μmol, Intermediate 554) to give 30.5 mg (95% purity, 27% yield) of the title compound.

LC-MS (Method 1): Rₜ=1.33 min; MS (ESIpos): m/z=502 [M+H]⁺

¹H-NMR (400 MHz, CHLOROFORM-d) δ [ppm]: 0.293 (0.44), 0.306 (0.65), 0.317 (0.76), 0.330 (0.86), 0.343 (0.65), 0.352 (0.54), 0.365 (0.72), 0.375 (0.52), 0.386 (0.42), 0.451 (0.62), 0.463 (0.75), 0.474 (0.67), 0.487 (0.46), 0.708 (0.58), 1.193 (0.45), 1.213 (0.53), 1.228 (0.44), 2.243 (0.61), 2.258 (0.68), 2.277 (0.91), 2.295 (0.57), 2.308 (0.52), 2.322 (0.48), 2.485 (1.33), 2.508 (1.26), 2.820 (0.45), 2.841 (0.51), 2.893 (0.80), 2.919 (0.94), 3.031 (1.01), 3.045 (0.95), 3.073 (0.53), 3.189 (1.02), 3.216 (0.92), 3.921 (16.00), 4.079 (0.45), 4.092 (1.41), 4.098 (1.47), 4.103 (2.62), 4.115 (1.96), 4.143 (0.49), 4.165 (1.99), 4.181 (2.55), 4.189 (1.00), 4.194 (1.13), 5.016 (3.26), 5.310 (5.59), 6.347 (2.37), 6.357 (3.51), 6.595 (1.17), 6.614 (1.97), 6.634 (0.85), 7.038 (1.23), 7.057 (1.45), 7.078 (0.92), 7.526 (1.06), 7.546 (1.28), 7.560 (0.81), 7.565 (1.00), 7.580 (0.88), 7.599 (0.65), 8.117 (2.38), 8.121 (2.41), 8.614 (2.18), 8.619 (2.22).

### Example 858

### 5-{(3R)-1-[1-(3-fluoropyridin-2-yl)propyl]-5′,6′-dihydrospiro[pyrrolidine-3,4′-pyrrolo[1,2-b]pyrazol]-2′-yl}-3-(trifluoromethyl)pyridin-2-amine (Diastereomer 1 and Diastereomer 2)

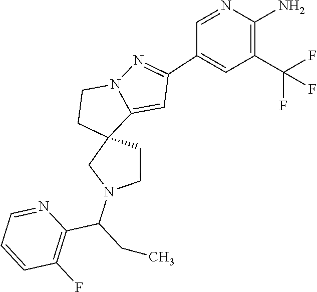 [SMILES: CCC(c1ncccc1F)N1CC[C@@]2(CCn3nc(-c4cnc(N)c(C(F)(F)F)c4)cc32)C1]

5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (150 mg, 95% purity, 396 μmol) and 1-(3-fluoropyridin-2-yl)propan-1-one (91.0 mg, 594 μmol) were dissolved in methanol (4.2 mL). N,N-Diisopropylethylamine (280 μL, 1.6 mmol), titanium(IV) isopropoxide (350 μL, 1.2 mmol) were added and stirred for 1 h at 60° C. To the cold reaction mixture was added sodium cyanoborohydride (74.7 mg, 1.19 mmol) and the mixture was stirred for 16 h at 60° C. The reaction mixture was allowed to cool down, diluted with water (0.5 mL) and stirred for 15 min. The suspension was filtered and the filter cake was washed with methanol. The filtrate was concentrated. The residue was purified by preparative HPLC to give 91.4 mg (100% purity, 50% yield) of the title compound.

LC-MS (Method 1): Rₜ=1.26 min; MS (ESIpos): m/z=461 [M+H]⁺

¹H-NMR (400 MHz, DMSO-d₆): δ [ppm]=0.75 (t, 3H), 1.82-2.03 (m, 4H), 2.37-2.48 (m, 1H), 2.54-3.00 (m, 4H), 3.93-4.00 (m, 1H), 4.07 (q, 2H), 6.22 and 6.31 (2s, 1H), 6.51 (s, 2H), 7.36-7.42 (m, 1H), 7.66-7.73 (m, 1H), 7.96-8.00 (m, 1H), 8.43-8.49 (m, 1H), 8.53-8.57 (m, 1H).

### Example 859

### 3-(difluoromethoxy)-5-{(3′R)-1′-[1-(1H-imidazol-2-yl)propyl]-6,7-dihydrospiro[pyrazolo[5,1-c][1,4]oxazine-4,3′-pyrrolidin]-2-yl}pyridin-2-amine (Diastereomer 1 and Diastereomer 2)

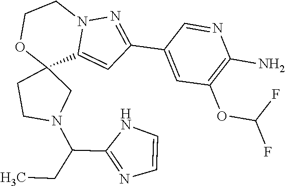 [SMILES: CCC(c1ncc[nH]1)N1CC[C@]2(C1)OCCn1nc(-c3cnc(N)c(OC(F)F)c3)cc12]

3-(Difluoromethoxy)-5-[(3′R)-6,7-dihydrospiro[pyrazolo[5,1-c][1,4]oxazine-4,3′-pyrrolidin]-2-yl]pyridin-2-amine (100 mg, 296 μmol, Intermediate 525) and 1-(1H-imidazol-2-yl)propan-1-one hydrogen chloride (1/1) (71.4 mg, 445 μmol, CAS 1314916-37-4) were dissolved in methanol (1 mL). N,N-Diisopropylethylamine (310 μL, 1.8 mmol), titanium(IV) isopropoxide (260 μL, 890 μmol) were added and the mixture was stirred for 20 h at 60° C. Sodium cyanoborohydride (55.9 mg, 889 μmol) was added and the mixture was stirred for 5 h at 60° C. The reaction mixture was allowed to cool down, diluted with water (1 mL) and stirred for 30 min. The suspension was diluted with methanol and filtered over celite. The filtrate was concentrated. The residue was diluted with dichloromethane/methanol (small amount of methanol) and washed with a saturated sodium bicarbonate solution. The aqueous phase was extracted with dichloromethane/methanol. The combined organic layers were dried over a hydrophobic filter and concentrated. The crude product was purified by column chromatography (silica gel, dichloromethane/methanol (0-7%) gradient) to give 78.0 mg (99% purity, 58% yield) of the title compound.

LC-MS (Method 1): Rₜ=0.83 min; MS (ESIpos): m/z=446 [M+H]⁺

¹H-NMR (400 MHz, CHLOROFORM-d) δ [ppm]: 0.740 (3.25), 0.759 (7.57), 0.777 (4.15), 0.794 (1.21), 0.811 (1.92), 0.820 (1.04), 1.035 (0.63), 1.072 (0.44), 1.087 (0.50), 1.185 (16.00), 1.210 (1.65), 1.215 (1.79), 1.264 (0.85), 1.267 (0.91), 1.614 (0.98), 1.727 (0.44), 1.745 (0.43), 1.890 (0.44), 1.903 (0.52), 1.922 (0.46), 1.978 (0.74), 2.150 (0.43), 2.165 (0.68), 2.184 (0.73), 2.201 (0.48), 2.231 (0.47), 2.248 (0.41), 2.736 (0.52), 2.764 (0.81), 2.783 (0.58), 2.801 (0.44), 2.861 (0.53), 2.877 (0.67), 3.150 (0.85), 3.177 (0.75), 3.542 (0.44), 3.997 (0.78), 4.009 (1.33), 4.021 (1.28), 4.027 (1.19), 4.034 (0.85), 4.039 (0.91), 4.080 (0.94), 4.092 (1.48), 4.096 (1.32), 4.108 (1.51), 4.113 (1.00), 4.120 (0.53), 4.125 (0.50), 4.728 (2.42), 5.233 (3.62), 6.170 (2.10), 6.184 (3.36), 6.302 (0.79), 6.306 (1.12), 6.485 (1.54), 6.489 (2.24), 6.669 (0.73), 6.672 (0.99), 6.935 (6.17), 6.945 (4.20), 7.620 (0.84), 7.630 (1.26), 8.213 (1.38), 8.218 (1.39), 8.223 (2.07), 8.227 (2.02).

### Example 860

### 3-(difluoromethoxy)-5-{(3′R)-1′-[1-(1H-imidazol-2-yl)propyl]-6,7-dihydrospiro[pyrazolo[5,1-c][1,4]oxazine-4,3′-pyrrolidin]-2-yl}pyridin-2-amine (Diastereomer 1)

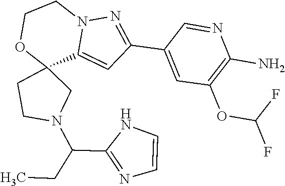 [SMILES: CCC(c1ncc[nH]1)N1CC[C@]2(C1)OCCn1nc(-c3cnc(N)c(OC(F)F)c3)cc12]

The compound 3-(difluoromethoxy)-5-{(3′R)-1′-[1-(1H-imidazol-2-yl)propyl]-6,7-dihydrospiro[pyrazolo[5,1-c][1,4]oxazine-4,3′-pyrrolidin]-2-yl}pyridin-2-amine (diastereomer 1 and diastereomer 2, Example 859) was separated into diastereomers by preparative chiral HPLC to give 19.5 mg (99% purity) of the title compound (diastereomer 1 Rₜ=4.0-5.1 min).

Preparative Chiral HPLC Method:

Instrument: Sepiatec: Prep SFC100; Column: Chiral Art Amylose SA 5μ, 250×50; eluent A: CO2; eluent B: 2-propanol+0.4 vol % diethylamine; isocratic: 30% B; flow: 100 mL/min; temperature: 40° C.; BPR: 150 bar; UV: 266 nm

Analytical Chiral HPLC Method:

Instrument: Waters Acquity UPC2 QDA; Column: Chiral Art Amylose SA 3μ, 100×4.6; eluent A: CO2; eluent B: 2-propanol+0.4 vol % diethylamine; isocratic: 30% B; flow: 4 mL/min; temperature: 37.5° C.; BPR: 100 bar; UV: 220 nm

Analytical chiral HPLC Rₜ=1.05 min

[α]²²_{D}: +57.1° (c=1.00, methanol)

LC-MS (Method 1): Rₜ=0.83 min; MS (ESIpos): m/z=446 [M+H]⁺

¹H-NMR (400 MHz, CHLOROFORM-d) δ [ppm]: −0.125 (1.54), 0.014 (14.96), 0.033 (16.00), 0.042 (3.78), 0.049 (0.84), 0.170 (1.54), 0.764 (5.47), 0.782 (12.42), 0.801 (5.71), 1.141 (0.20), 1.160 (2.83), 1.175 (2.73), 1.212 (2.53), 1.668 (1.79), 1.723 (0.89), 1.741 (0.94), 1.746 (0.89), 1.757 (1.09), 1.764 (0.94), 1.775 (0.99), 1.780 (1.09), 1.798 (0.84), 1.817 (0.30), 1.896 (0.30), 1.914 (0.84), 1.926 (0.99), 1.933 (0.94), 1.945 (1.04), 1.948 (0.89), 1.959 (0.75), 1.967 (0.75), 1.978 (0.65), 1.997 (0.20), 2.129 (0.65), 2.135 (0.94), 2.151 (0.60), 2.166 (0.75), 2.171 (0.84), 2.185 (1.49), 2.200 (1.34), 2.206 (1.24), 2.221 (1.09), 2.277 (0.80), 2.296 (1.29), 2.312 (1.44), 2.328 (0.80), 2.346 (0.55), 2.573 (3.53), 2.584 (0.75), 2.591 (0.60), 2.600 (0.89), 2.627 (0.94), 2.643 (0.65), 2.853 (1.34), 2.880 (4.57), 2.897 (4.17), 2.923 (1.09), 3.053 (0.80), 3.073 (1.29), 3.091 (1.14), 3.111 (0.65), 3.369 (0.20), 3.524 (1.44), 3.536 (1.59), 3.548 (1.54), 3.558 (1.39), 3.978 (0.25), 3.992 (0.45), 4.003 (0.70), 4.007 (1.34), 4.021 (3.63), 4.033 (5.12), 4.045 (2.34), 4.063 (0.55), 4.074 (0.75), 4.094 (0.65), 4.103 (4.02), 4.115 (6.16), 4.129 (1.94), 4.747 (5.81), 6.191 (12.47), 6.325 (3.83), 6.508 (7.20), 6.692 (3.43), 6.954 (0.75), 6.968 (8.99), 7.476 (0.40), 7.640 (3.78), 7.641 (4.02), 7.644 (3.88), 8.237 (6.31), 8.241 (6.16), 9.363 (0.55).

### Example 861

### 3-(difluoromethoxy)-5-{(3′R)-1′-[1-(1H-imidazol-2-yl)propyl]-6,7-dihydrospiro[pyrazolo[5,1-c][1,4]oxazine-4,3′-pyrrolidin]-2-yl}pyridin-2-amine (Diastereomer 2)

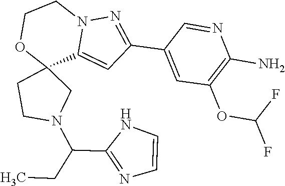 [SMILES: CCC(c1ncc[nH]1)N1CC[C@]2(C1)OCCn1nc(-c3cnc(N)c(OC(F)F)c3)cc12]

For the preparation of the diastereomeric mixture of the title compound see Example 859. The diastereomers were separated by preparative chiral HPLC (method see Example 860) to give 28.2 mg (99% purity) of the title compound (diastereomer 2 Rₜ=7.3-9.5 min).

Analytical chiral HPLC (for method see Example 860): Rₜ=1.97 min

[α]²²_{D}: +58.3° (c=1.00, methanol)

LC-MS (Method 1): Rₜ=0.83 min; MS (ESIpos): m/z=446 [M+H]⁺

¹H-NMR (400 MHz, CHLOROFORM-d) δ [ppm]: −0.108 (0.54), 0.016 (0.74), 0.032 (5.11), 0.050 (5.44), 0.058 (1.24), 0.065 (0.21), 0.187 (0.54), 0.782 (1.44), 0.801 (3.37), 0.819 (1.52), 1.229 (0.16), 1.623 (1.52), 1.724 (0.16), 1.743 (0.21), 1.747 (0.20), 1.759 (0.26), 1.777 (0.25), 1.781 (0.26), 1.799 (0.20), 1.909 (0.23), 1.921 (0.25), 1.928 (0.25), 1.940 (0.28), 1.943 (0.25), 1.955 (0.20), 1.962 (0.20), 1.973 (0.16), 2.187 (0.23), 2.201 (0.34), 2.221 (0.46), 2.238 (0.31), 2.251 (0.23), 2.268 (0.43), 2.284 (0.36), 2.301 (0.20), 2.589 (16.00), 2.770 (0.85), 2.797 (1.21), 2.816 (0.84), 2.832 (0.57), 2.851 (0.29), 3.184 (0.77), 3.210 (0.67), 3.574 (0.38), 3.586 (0.43), 3.597 (0.41), 3.609 (0.38), 4.047 (0.38), 4.054 (0.44), 4.059 (0.74), 4.066 (0.87), 4.073 (0.69), 4.078 (0.70), 4.136 (0.84), 4.148 (1.11), 4.153 (0.79), 4.160 (0.43), 4.165 (0.41), 4.766 (1.52), 6.221 (3.49), 6.346 (0.92), 6.530 (1.88), 6.713 (0.92), 6.970 (1.05), 6.980 (1.05), 7.669 (1.00), 7.671 (1.05), 7.674 (1.00), 8.264 (1.60), 8.269 (1.67), 9.353 (0.18).

### Example 862

### 2-amino-5-{(3′R)-1′-[1-(1H-imidazol-2-yl)propyl]-6,7-dihydrospiro[pyrazolo[5,1-c][1,4]oxazine-4,3′-pyrrolidin]-2-yl}pyridine-3-carbonitrile (Diastereomer 1 and Diastereomer 2)

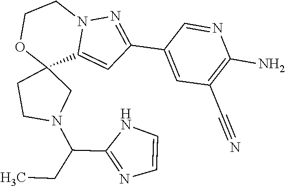 [SMILES: CCC(c1ncc[nH]1)N1CC[C@]2(C1)OCCn1nc(-c3cnc(N)c(C#N)c3)cc12]

The compound was prepared similarly to Example 859 using 2-amino-5-[(3′R)-6,7-dihydrospiro[pyrazolo[5,1-c][1,4]oxazine-4,3′-pyrrolidin]-2-yl]pyridine-3-carbonitrile (100 mg, 337 μmol, Intermediate 524) and 1-(1H-imidazol-2-yl)propan-1-one hydrogen chloride (1/1) (81.3 mg, 506 μmol, CAS 1314916-37-4) to give 88.0 mg (99% purity, 64% yield) of the title compound.

LC-MS (Method 1): Rₜ=0.77 min; MS (ESIpos): m/z=405 [M+H]⁺

¹H-NMR (400 MHz, CHLOROFORM-d) δ [ppm]: 0.740 (6.19), 0.759 (14.18), 0.777 (6.87), 0.811 (0.87), 1.184 (3.86), 1.209 (1.03), 1.267 (0.52), 1.353 (0.48), 1.697 (0.54), 1.732 (1.06), 1.741 (0.92), 1.750 (1.10), 1.766 (0.82), 1.783 (0.59), 1.876 (0.88), 1.887 (0.99), 1.895 (1.17), 1.907 (1.34), 1.928 (1.18), 1.940 (1.00), 1.959 (0.61), 1.978 (1.29), 2.120 (0.81), 2.141 (1.16), 2.155 (1.92), 2.175 (1.89), 2.191 (1.38), 2.221 (0.75), 2.237 (1.22), 2.254 (1.17), 2.275 (1.02), 2.295 (0.89), 2.312 (0.55), 2.329 (0.42), 2.571 (0.41), 2.592 (0.73), 2.729 (1.83), 2.756 (2.10), 2.771 (1.00), 2.789 (1.70), 2.807 (1.33), 2.833 (1.07), 2.861 (1.84), 2.881 (1.94), 2.908 (0.64), 3.040 (0.41), 3.059 (0.69), 3.078 (0.62), 3.160 (2.11), 3.186 (1.83), 3.517 (0.68), 3.528 (0.76), 3.540 (0.77), 3.552 (1.47), 3.564 (1.07), 3.576 (1.02), 3.587 (0.89), 3.989 (0.71), 4.002 (1.86), 4.014 (3.04), 4.027 (3.10), 4.033 (2.80), 4.039 (1.80), 4.045 (2.81), 4.062 (0.57), 4.080 (1.94), 4.093 (3.65), 4.108 (3.50), 4.120 (1.29), 4.125 (1.21), 5.188 (6.38), 5.233 (13.90), 6.177 (5.05), 6.189 (7.52), 6.929 (0.54), 6.944 (16.00), 6.952 (11.33), 8.012 (2.76), 8.018 (3.12), 8.021 (4.22), 8.027 (4.08), 8.554 (2.91), 8.560 (3.10), 8.564 (4.45), 8.570 (4.20).

### Example 863

### 2-amino-5-{(3′R)-1′-[1-(1H-imidazol-2-yl)propyl]-6,7-dihydrospiro[pyrazolo[5,1-c][1,4]oxazine-4,3′-pyrrolidin]-2-yl}pyridine-3-carbonitrile (Diastereomer 1)

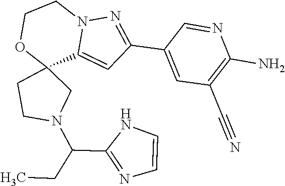 [SMILES: CCC(c1ncc[nH]1)N1CC[C@]2(C1)OCCn1nc(-c3cnc(N)c(C#N)c3)cc12]

The compound 2-amino-5-{(3′R)-1′-[1-(1H-imidazol-2-yl)propyl]-6,7-dihydrospiro[pyrazolo[5,1-c][1,4]oxazine-4,3′-pyrrolidin]-2-yl}pyridine-3-carbonitrile (diastereomer 1 and diastereomer 2, Example 862) was separated into diastereomers by preparative chiral HPLC to give 22.2 mg (98% purity) of the title compound (diastereomer 1 Rₜ=5.45-6.4 min).

Preparative Chiral HPLC Method:

Instrument: sepiatec: Prep SFC100; Column: Chiralpak IG 5μ, 250×50; eluent A: CO2; eluent B: methanol+0.2% aqueous ammonia (32%); isocratic: 40% B; flow: 100 mL/min; temperature: 40° C.; BPR: 150 bar UV: 210 nm

Analytical Chiral HPLC Method:

Instrument: Waters Acquity UPC2 QDA; Column: Chiralpak IG 3μ, 100×4.6; eluent A: CO2; eluent B: methanol+0.2% aqueous ammonia (32%); isocratic: 40% B; flow: 4 mL/min; temperature: 40° C.; BPR: 100 bar UV: 220 nm

Analytical chiral HPLC Rₜ=1.54 min

[α]²²_{D}: +64.9° (c=1.00, methanol)

LC-MS (Method 1): Rₜ=0.77 min; MS (ESIpos): m/z=405 [M+H]⁺

1H-NMR (400 MHz, CHLOROFORM-d) δ [ppm]: −0.124 (0.81), 0.015 (8.66), 0.033 (9.39), 0.042 (1.96), 0.050 (0.50), 0.170 (0.83), 0.763 (6.99), 0.782 (16.00), 0.801 (7.30), 1.213 (0.98), 1.731 (0.98), 1.750 (1.29), 1.755 (1.31), 1.765 (1.62), 1.784 (1.67), 1.788 (1.76), 1.807 (1.45), 1.899 (0.48), 1.918 (1.22), 1.929 (1.36), 1.936 (1.31), 1.947 (1.48), 1.951 (1.26), 1.963 (1.05), 1.970 (0.98), 1.981 (0.83), 2.144 (0.79), 2.159 (0.98), 2.165 (1.17), 2.178 (1.96), 2.193 (1.69), 2.199 (1.65), 2.214 (1.36), 2.282 (1.00), 2.299 (1.62), 2.315 (1.79), 2.332 (1.03), 2.351 (0.72), 2.584 (0.86), 2.600 (1.10), 2.643 (0.74), 2.850 (1.17), 2.877 (3.31), 2.901 (4.32), 2.927 (1.45), 3.057 (1.00), 3.076 (1.65), 3.096 (1.45), 3.115 (0.79), 3.529 (1.50), 3.539 (1.69), 3.551 (1.65), 3.563 (1.48), 3.994 (0.43), 3.997 (0.45), 4.013 (1.62), 4.025 (4.41), 4.037 (5.79), 4.044 (1.55), 4.049 (2.89), 4.067 (0.52), 4.073 (0.67), 4.078 (0.86), 4.104 (4.10), 4.116 (7.23), 4.127 (2.31), 4.130 (2.34), 5.231 (7.77), 6.198 (11.66), 6.955 (0.57), 6.973 (12.35), 8.035 (7.96), 8.041 (8.11), 8.577 (8.49), 8.584 (8.35), 9.453 (0.45).

### Example 864

### 2-amino-5-{(3′R)-1′-[1-(1H-imidazol-2-yl)propyl]-6,7-dihydrospiro[pyrazolo[5,1-c][1,4]oxazine-4,3′-pyrrolidin]-2-yl}pyridine-3-carbonitrile (Diastereomer 2)

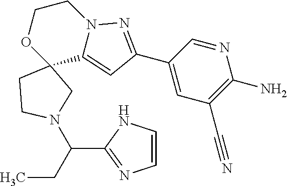 [SMILES: CCC(c1ncc[nH]1)N1CC[C@]2(C1)OCCn1nc(-c3cnc(N)c(C#N)c3)cc12]

For the preparation of the diastereomeric mixture of the title compound see Example 862. The diastereomers were separated by preparative chiral HPLC (method see Example 863) to give 29.1 mg (99% purity) of the title compound (diastereomer 2 Rₜ=7.14-8.20 min).

Analytical chiral HPLC (method see Example 863): Rₜ=2.16 min

[α]²²_{D}: +75.0° (c=1.00, methanol)

LC-MS (Method 1): Rₜ=0.77 min; MS (ESIpos): m/z=405 [M+H]⁺

¹H-NMR (400 MHz, CHLOROFORM-d) δ [ppm]: 0.320 (0.62), 0.330 (16.00), 0.339 (0.53), 1.060 (0.97), 1.079 (2.26), 1.097 (1.03), 2.056 (0.20), 2.064 (0.17), 2.074 (0.20), 2.080 (0.22), 2.098 (0.20), 2.161 (0.32), 2.195 (0.20), 2.202 (0.27), 2.213 (0.26), 2.221 (0.24), 2.232 (0.25), 2.235 (0.22), 2.247 (0.17), 2.463 (0.18), 2.477 (0.25), 2.497 (0.30), 2.514 (0.20), 2.561 (0.31), 2.578 (0.26), 2.595 (0.16), 3.036 (0.64), 3.062 (0.75), 3.086 (0.27), 3.097 (0.34), 3.103 (0.51), 3.112 (0.34), 3.117 (0.37), 3.132 (0.22), 3.498 (0.55), 3.525 (0.49), 3.863 (0.28), 3.874 (0.32), 3.886 (0.30), 3.898 (0.27), 4.343 (0.26), 4.355 (0.54), 4.361 (0.65), 4.368 (0.42), 4.373 (0.62), 4.425 (0.58), 4.437 (0.76), 4.442 (0.58), 4.449 (0.29), 4.454 (0.28), 5.626 (1.24), 6.512 (2.41), 7.525 (3.13), 8.347 (1.16), 8.353 (1.18), 8.891 (1.25), 8.896 (1.21).

### Example 865

### 3-(difluoromethyl)-5-{(3′R)-1′-[1-(1H-imidazol-2-yl)propyl]-6,7-dihydrospiro[pyrazolo[5,1-c][1,4]oxazine-4,3′-pyrrolidin]-2-yl}pyridin-2-amine (Diastereomer 1 and Diastereomer 2)

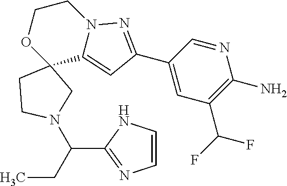 [SMILES: CCC(c1ncc[nH]1)N1CC[C@]2(C1)OCCn1nc(-c3cnc(N)c(C(F)F)c3)cc12]

The compound was prepared similarly to Example 859 using 3-(difluoromethyl)-5-[(3′R)-6,7-dihydrospiro[pyrazolo[5,1-c][1,4]oxazine-4,3′-pyrrolidin]-2-yl]pyridin-2-amine (130 mg, 405 μmol, Intermediate 523) and 1-(1H-imidazol-2-yl)propan-1-one hydrogen chloride (1/1) (97.5 mg, 607 μmol, CAS 1314916-37-4) to give 65.0 mg (95% purity, 36% yield) of the title compound.

LC-MS (Method 1): Rₜ=0.81 min; MS (ESIpos): m/z=430 [M+H]⁺

¹H-NMR (400 MHz, CHLOROFORM-d) 6 [ppm]: 0.009 (16.00), 0.018 (0.68), 0.752 (4.86), 0.770 (11.02), 0.789 (5.26), 0.820 (0.61), 1.193 (1.63), 1.206 (1.10), 1.354 (2.74), 1.388 (2.40), 1.407 (4.08), 1.426 (2.01), 1.721 (0.43), 1.740 (0.62), 1.755 (0.85), 1.774 (0.87), 1.795 (0.67), 1.809 (0.40), 1.894 (0.70), 1.905 (0.83), 1.912 (0.98), 1.923 (1.11), 1.946 (0.94), 1.957 (0.81), 1.965 (0.61), 1.975 (0.45), 2.150 (0.52), 2.166 (0.83), 2.184 (1.18), 2.194 (0.79), 2.202 (1.40), 2.215 (0.69), 2.222 (0.94), 2.237 (0.76), 2.254 (1.13), 2.270 (1.10), 2.289 (0.96), 2.309 (0.75), 2.325 (0.47), 2.633 (0.61), 2.647 (0.59), 2.770 (2.04), 2.797 (2.85), 2.814 (1.46), 2.832 (1.17), 2.852 (0.91), 2.867 (0.80), 2.889 (0.43), 2.912 (2.61), 2.954 (0.69), 2.972 (1.70), 2.991 (1.66), 3.009 (0.61), 3.086 (0.57), 3.106 (0.50), 3.183 (1.80), 3.210 (1.58), 3.291 (0.88), 3.486 (0.43), 3.503 (0.94), 3.519 (1.22), 3.536 (0.91), 3.553 (0.43), 3.565 (0.53), 3.576 (0.58), 3.588 (0.63), 3.598 (1.26), 3.609 (1.08), 3.620 (1.00), 3.631 (0.89), 3.999 (0.57), 4.012 (1.33), 4.024 (2.41), 4.036 (2.61), 4.042 (2.44), 4.049 (1.77), 4.054 (1.93), 4.084 (0.62), 4.094 (1.53), 4.107 (2.63), 4.111 (2.80), 4.122 (3.13), 4.127 (2.17), 4.134 (1.21), 4.139 (1.16), 4.929 (4.21), 5.242 (0.85), 6.213 (3.86), 6.219 (7.09), 6.422 (1.45), 6.560 (2.89), 6.698 (1.39), 6.938 (0.46), 6.963 (13.56), 6.970 (7.48), 7.878 (2.77), 8.470 (2.66).

### Example 866

### 3-(difluoromethyl)-5-{(3′R)-1′-[1-(1H-imidazol-2-yl)propyl]-6,7-dihydrospiro[pyrazolo[5,1-c][1,4]oxazine-4,3′-pyrrolidin]-2-yl}pyridin-2-amine (Diastereomer 1)

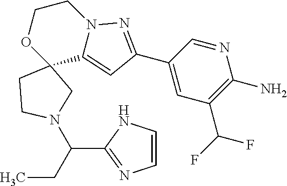 [SMILES: CCC(c1ncc[nH]1)N1CC[C@]2(C1)OCCn1nc(-c3cnc(N)c(C(F)F)c3)cc12]

The compound 3-(difluoromethyl)-5-{(3′R)-1′-[1-(1H-imidazol-2-yl)propyl]-6,7-dihydrospiro[pyrazolo[5,1-c][1,4]oxazine-4,3′-pyrrolidin]-2-yl}pyridin-2-amine (diastereomer 1 and diastereomer 2, Example 865) was separated into diastereomers by preparative chiral HPLC to give 11.0 mg (90% purity) of the title compound (diastereomer 1 Rₜ=4.3-5.8 min).

Preparative Chiral HPLC Method:

Instrument: Sepiatec: Prep SFC100; Column: Chiral Art Amylose SA 5μ, 250×50; eluent A: CO2; eluent B: 2-propanol+0.4 vol % diethylamine; isocratic: 30% B; flow: 100 mL/min; temperature: 40° C.; BPR: 150 bar; UV: 254 nm

Analytical Chiral HPLC Method:

Instrument: Waters Acquity UPC2 QDA; Column: Chiral Art Amylose SA 3μ, 100×4.6; eluent A: CO2; eluent B: 2-propanol+0.4 vol % diethylamine; isocratic: 30% B; flow: 4 mL/min; temperature: 40° C.; BPR: 1800 psi; UV: 254 nm

Analytical chiral HPLC Rₜ=1.12 min

[α]²²_{D}: +59.4° (c=1.00, methanol)

LC-MS (Method 1): Rₜ=0.81 min; MS (ESIpos): m/z=430 [M+H]⁺

¹H-NMR (400 MHz, CHLOROFORM-d) δ [ppm]: −0.125 (1.33), 0.014 (12.69), 0.033 (13.82), 0.042 (3.03), 0.170 (1.33), 0.764 (6.02), 0.782 (13.98), 0.801 (6.46), 1.198 (0.48), 1.212 (0.40), 1.709 (0.77), 1.727 (1.17), 1.745 (1.33), 1.750 (1.29), 1.761 (1.49), 1.779 (1.41), 1.784 (1.49), 1.803 (1.21), 1.821 (0.61), 1.899 (0.44), 1.917 (1.05), 1.928 (1.17), 1.936 (1.13), 1.947 (1.25), 1.963 (0.93), 1.970 (0.89), 1.981 (0.77), 2.155 (0.65), 2.170 (0.85), 2.175 (0.97), 2.190 (1.70), 2.204 (1.45), 2.210 (1.41), 2.225 (1.25), 2.283 (0.93), 2.301 (1.49), 2.317 (1.62), 2.334 (0.97), 2.351 (0.69), 2.586 (0.77), 2.645 (0.73), 2.856 (1.33), 2.883 (4.16), 2.902 (4.28), 2.929 (1.29), 3.057 (0.85), 3.076 (1.45), 3.096 (1.33), 3.115 (0.73), 3.300 (0.53), 3.303 (0.53), 3.526 (1.49), 3.537 (1.66), 3.549 (1.58), 3.560 (1.45), 3.997 (0.53), 4.013 (1.54), 4.026 (4.08), 4.038 (5.29), 4.051 (2.59), 4.068 (0.89), 4.079 (1.05), 4.089 (0.40), 4.099 (0.97), 4.108 (4.32), 4.120 (6.71), 4.131 (2.30), 4.932 (6.42), 6.210 (11.96), 6.230 (0.44), 6.430 (2.55), 6.567 (5.17), 6.605 (0.73), 6.705 (2.38), 6.954 (0.61), 6.971 (16.00), 7.476 (0.40), 7.886 (3.88), 8.472 (3.68), 8.474 (3.80), 8.477 (3.72).

### Example 867

### 3-(difluoromethyl)-5-{(3′R)-1′-[1-(1H-imidazol-2-yl)propyl]-6,7-dihydrospiro[pyrazolo[5,1-c][1,4]oxazine-4,3′-pyrrolidin]-2-yl}pyridin-2-amine (Diastereomer 2)

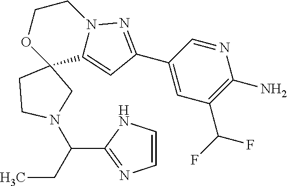 [SMILES: CCC(c1ncc[nH]1)N1CC[C@]2(C1)OCCn1nc(-c3cnc(N)c(C(F)F)c3)cc12]

For the preparation of the diastereomeric mixture of the title compound see Example 865. The diastereomers were separated by preparative chiral HPLC (method see Example 866) to give 21.0 mg (95% purity) of the title compound (diastereomer 2 Rₜ=7.6-9.9 min).

Analytical chiral HPLC (method see Example 866): Rₜ=2.08 min

[α]²²_{D}: +147.9° (c=1.00, methanol)

LC-MS (Method 1): Rₜ=0.81 min; MS (ESIpos): m/z=430 [M+H]⁺

¹H-NMR (400 MHz, CHLOROFORM-d) δ [ppm]: −0.111 (1.04), 0.014 (1.63), 0.029 (11.24), 0.047 (9.21), 0.056 (2.00), 0.184 (1.01), 0.776 (6.99), 0.795 (16.00), 0.813 (7.44), 1.728 (2.20), 1.747 (3.15), 1.751 (3.10), 1.762 (3.10), 1.781 (2.20), 1.785 (2.17), 1.803 (1.49), 1.821 (0.56), 1.891 (0.42), 1.909 (1.15), 1.921 (1.32), 1.928 (1.27), 1.940 (1.49), 1.943 (1.24), 1.955 (1.01), 1.962 (0.93), 1.973 (0.82), 2.166 (0.68), 2.186 (1.15), 2.201 (1.83), 2.220 (2.23), 2.237 (1.55), 2.253 (1.21), 2.270 (2.25), 2.286 (1.89), 2.304 (1.04), 2.320 (0.59), 2.766 (4.45), 2.792 (6.08), 2.814 (4.48), 2.832 (3.07), 2.849 (1.58), 3.192 (4.00), 3.219 (3.55), 3.571 (2.11), 3.583 (2.34), 3.594 (2.23), 3.605 (1.97), 4.033 (0.48), 4.047 (1.97), 4.060 (3.83), 4.068 (4.42), 4.073 (3.41), 4.079 (3.38), 4.108 (0.73), 4.137 (4.31), 4.149 (5.94), 4.153 (3.86), 4.160 (2.17), 4.165 (2.06), 4.987 (7.58), 6.233 (15.38), 6.451 (2.99), 6.588 (6.00), 6.726 (2.85), 6.974 (10.90), 7.907 (4.73), 8.500 (4.68), 8.502 (4.73), 8.505 (4.39), 9.532 (0.65).

### Example 868

### (3R)-2′-(6-amino-5-{[1-(1-propyl-1H-pyrazol-5-yl)ethyl]oxy}pyridin-3-yl)-N-ethyl-5′,6′-dihydrospiro[pyrrolidine-3,4′-pyrrolo[1,2-b]pyrazole]-1-carboxamide (Diastereomer 1 and Diastereomer 2)

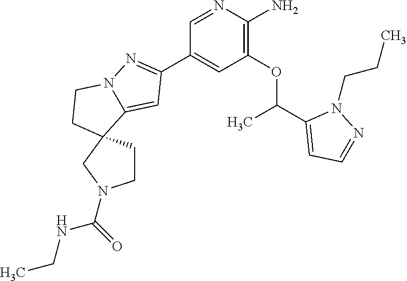 [SMILES: CCCn1nccc1C(C)Oc1cc(-c2cc3n(n2)CC[C@@]32CCN(C(=O)NCC)C2)cnc1N]

3-{[1-(1-Propyl-1H-pyrazol-5-yl)ethyl]oxy}-5-(4,4,5,5-tetramethyl-1,3,2-dioxaborolan-2-yl)pyridin-2-amine (91.0 mg, 244 μmol), (3R)-1-(ethylcarbamoyl)-5′,6′-dihydrospiro[pyrrolidine-3,4′-pyrrolo[1,2-b]pyrazol]-2′-yl trifluoromethanesulfonate (140 mg, 367 μmol) and potassium phosphate (1.5 mL, 0.50 M, 730 μmol) were dissolved in degassed 1,4-dioxane. XPhos Pd G2 (9.60 mg, 12.2 μmol) was added and the mixture was stirred for 2 h at 100° C. The cold reaction mixture was diluted with water and extracted with ethyl acetate. The combined organic layers were washed with brine, dried over sodium sulfate, filtered and concentrated. The residue was purified by preparative HPLC followed by preparative TLC (dichloromethane/methanol 9:1 gradient) to give 5.00 mg (98% purity, 4% yield) of the title compound.

LC-MS (Method 1): Rₜ=0.88 min; MS (ESIpos): m/z=480 [M+H]⁺

¹H NMR (DMSO-d₆, 400 MHz): δ (ppm) 7.92 (d, 1H), 7.41 (d, 1H), 7.37 (d, 1H), 6.36 (d, 1H), 6.28 (d, 1H), 6.16 (t, 1H), 5.81 (q, 1H), 5.75 (s, 2H), 4.15 (t, 2H), 4.03-4.08 (m, 2H), 3.45-3.52 (m, 1H), 3.35-3.43 (m, 3H), 3.01-3.09 (m, 2H), 1.98-2.10 (m, 2H), 1.73 (sxt, 2H), 1.62 (d, 3H), 1.02 (t, 3H), 0.80 (2t, 3H).

### Example 869

### (3R)-2′-(6-amino-5-{[1-(1,3-thiazol-4-yl)ethyl]oxy}pyridin-3-yl)-N-ethyl-5′,6′-dihydrospiro[pyrrolidine-3,4′-pyrrolo[1,2-b]pyrazole]-1-carboxamide (Diastereomer 1 and Diastereomer 2)

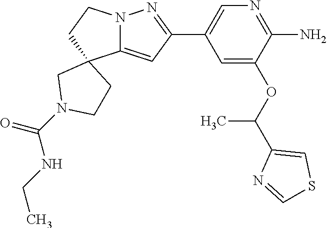 [SMILES: CCNC(=O)N1CC[C@@]2(CCn3nc(-c4cnc(N)c(OC(C)c5cscn5)c4)cc32)C1]

5-(4,4,5,5-Tetramethyl-1,3,2-dioxaborolan-2-yl)-3-{[1-(1,3-thiazol-4-yl)ethyl]oxy}pyridin-2-amine (285 mg, 36% purity, 295 μmol) was dissolved in 1,4-dioxane (1.7 mL) under argon. (3R)-1-(Ethylcarbamoyl)-5′,6′-dihydrospiro[pyrrolidine-3,4′-pyrrolo[1,2-b]pyrazol]-2′-yl trifluoromethanesulfonate (94.1 mg, 246 μmol), potassium phosphate (1.5 mL, 0.50 M, 740 μmol) and XPhos Pd G2 (9.69 mg, 12.3 μmol) were added and the mixture was stirred for 1 h at 100° C. The reaction mixture was allowed to cool down, diluted with dichloromethane, washed with water and brine. The organic phase was dried over a hydrophobic filter and concentrated. The residue was purified by preparative HPLC to give 15.3 mg (95% purity, 13% yield) of the title compound.

LC-MS (Method 1): Rₜ=0.82 min; MS (ESIpos): m/z=454 [M+H]⁺

¹H-NMR (400 MHz, DMSO-d₆): δ [ppm]=1.02 (t, 3H), 1.65 (d, 3H), 1.97-2.10 (m, 2H), 3.00-3.10 (m, 2H), 3.35-3.42 (m, 3H), 3.45-3.52 (m, 1H), 4.14 (t, 2H), 5.70 (q, 1H), 5.80 (s, 2H), 6.15 (t, 1H), 6.30 (d, 1H), 7.38 (s, 1H), 7.73 (d, 1H), 7.92 (d, 1H), 9.10 (d, 1H).

### Example 870

### (3R)-2′-(6-amino-5-{[1-(pyrimidin-4-yl)ethyl]oxy}pyridin-3-yl)-N-(propan-2-yl)-5′,6′-dihydrospiro[pyrrolidine-3,4′-pyrrolo[1,2-b]pyrazole]-1-carboxamide (Diastereomer 1 and Diastereomer 2)

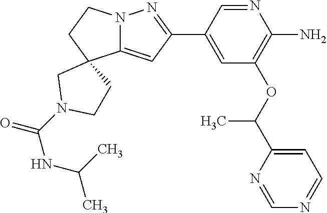 [SMILES: CC(C)NC(=O)N1CC[C@@]2(CCn3nc(-c4cnc(N)c(OC(C)c5ccncn5)c4)cc32)C1]

(3R)-1-[(Propan-2-yl)carbamoyl]-5′,6′-dihydrospiro[pyrrolidine-3,4′-pyrrolo[1,2-b]pyrazol]-2′-yl trifluoromethanesulfonate (146 mg, 369 μmol), 3-{[1-(pyrimidin-4-yl)ethyl]oxy}-5-(4,4,5,5-tetramethyl-1,3,2-dioxaborolan-2-yl)pyridin-2-amine (139 mg, 406 μmol), potassium phosphate (2.2 mL, 0.50M, 1.10 mmol) and Xphos Pd G2 (14.5 mg, 18.5 μmol) were combined in degassed 1,4-dioxane (6.1 mL) and the mixture was stirred for 80 min at 100° C. The reaction mixture was allowed to cooled down and diluted with water and dichloromethane. The layers were separated and the aqueous phase was extracted with dichloromethane/methanol 9:1. The combined organic layers were dried and concentrated. The residue was purified by column chromatography (silica gel, dichloromethane/methanol (0-10%) gradient) to give 106 mg (90% purity, 56% yield) of the title compound.

LC-MS (Method 1): Rₜ=0.80 min; MS (ESIpos): m/z=463 [M+H]⁺

¹H NMR (DMSO-d₆, 400 MHz): δ (ppm) 9.18 (t, 1H), 8.81 (dd, 1H), 7.91 (d, 1H), 7.67 (dt, 1H), 7.24 (t, 1H), 6.26 (s, 1H), 5.97 (s, 2H), 5.81 (d, 1H), 5.55 (q, 1H), 4.12 (t, 2H), 3.69-3.82 (m, 1H), 3.44-3.52 (m, 1H), 3.35-3.41 (m, 3H), 1.95-2.08 (m, 2H), 1.62 (d, 3H), 1.06 and 1.07 (2d, 6H).

### Example 871

### (3R)-2′-(6-amino-5-{[1-(2,4-dimethylpyrimidin-5-yl)ethyl]oxy}pyridin-3-yl)-N-(propan-2-yl)-5′,6′-dihydrospiro[pyrrolidine-3,4′-pyrrolo[1,2-b]pyrazole]-1-carboxamide (Diastereomer 1)

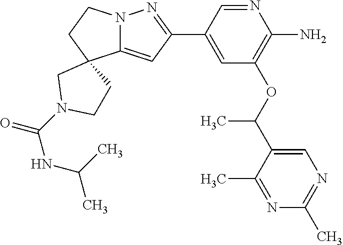 [SMILES: Cc1ncc(C(C)Oc2cc(-c3cc4n(n3)CC[C@@]43CCN(C(=O)NC(C)C)C3)cnc2N)c(C)n1]

The compound was prepared similarly to Example 870 using (3R)-1-[(propan-2-yl)carbamoyl]-5′,6′-dihydrospiro[pyrrolidine-3,4′-pyrrolo[1,2-b]pyrazol]-2′-yl trifluoromethanesulfonate (88.6 mg, 223 μmol) and 3-{[1-(2,4-dimethylpyrimidin-5-yl)ethyl]oxy}-5-(4,4,5,5-tetramethyl-1,3,2-dioxaborolan-2-yl)pyridin-2-amine (enantiomer 1) (91.0 mg, 246 μmol) to give 65.0 mg (90% purity, 53% yield) of the title compound.

[α]²⁰_{D}: −14.3° (c=1.00, methanol)

LC-MS (Method 1): Rₜ=0.84 min; MS (ESIpos): m/z=491 [M+H]⁺

¹H-NMR (500 MHz, DMSO-d₆): δ [ppm]=1.05-1.08 (m, 6H), 1.60 (d, 3H), 1.97-2.09 (m, 2H), 2.51 (br s, 3H), 2.54 (s, 3H), 3.34-3.41 (m, 3H), 3.46-3.52 (m, 1H), 3.71-3.79 (m, 1H), 4.11-4.16 (m, 2H), 5.77-5.83 (m, 2H), 5.93 (s, 2H), 6.27 (s, 1H), 7.28 (d, 1H), 7.88 (d, 1H), 8.72 (s, 1H).

### Example 872

### (3R)-2′-(6-amino-5-{[1-(2,4-dimethylpyrimidin-5-yl)ethyl]oxy}pyridin-3-yl)-N-(propan-2-yl)-5′,6′-dihydrospiro[pyrrolidine-3,4′-pyrrolo[1,2-b]pyrazole]-1-carboxamide (Diastereomer 2)

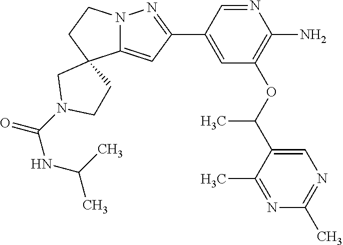 [SMILES: Cc1ncc(C(C)Oc2cc(-c3cc4n(n3)CC[C@@]43CCN(C(=O)NC(C)C)C3)cnc2N)c(C)n1]

The compound was prepared similarly to Example 870 using (3R)-1-[(propan-2-yl)carbamoyl]-5′,6′-dihydrospiro[pyrrolidine-3,4′-pyrrolo[1,2-b]pyrazol]-2′-yl trifluoromethanesulfonate (88.6 mg, 223 μmol) and 3-{[1-(2,4-dimethylpyrimidin-5-yl)ethyl]oxy}-5-(4,4,5,5-tetramethyl-1,3,2-dioxaborolan-2-yl)pyridin-2-amine (enantiomer 2) (91.0 mg, 246 μmol) to give 67.0 mg (90% purity, 55% yield) of the title compound.

[α]²⁰_{D}: +106.3° (c=1.00, methanol)

LC-MS (Method 1): Rₜ=0.84 min; MS (ESIpos): m/z=491 [M+H]⁺

¹H-NMR (500 MHz, DMSO-d₆): δ [ppm]=1.04-1.08 (m, 6H), 1.60 (d, 3H), 1.98-2.09 (m, 2H), 2.51 (s, 3H), 2.54 (s, 3H), 3.35-3.42 (m, 3H), 3.49 (dq, 1H), 3.71-3.80 (m, 1H), 4.14 (t, 2H), 5.77-5.83 (m, 2H), 5.93 (s, 2H), 6.27 (s, 1H), 7.28 (d, 1H), 7.88 (d, 1H), 8.73 (s, 1H).

### Example 873

### (3R)-2′-{6-amino-5-[(1R)-1-(5-methyl-1,3,4-oxadiazol-2-yl)ethoxy]pyridin-3-yl}-N-(propan-2-yl)-5′,6′-dihydrospiro[pyrrolidine-3,4′-pyrrolo[1,2-b]pyrazole]-1-carboxamide

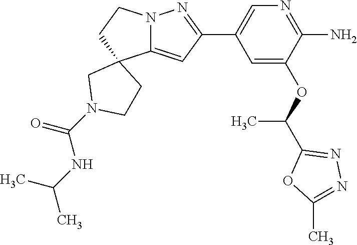 [SMILES: Cc1nnc([C@@H](C)Oc2cc(-c3cc4n(n3)CC[C@@]43CCN(C(=O)NC(C)C)C3)cnc2N)o1]

3-[(1R)-1-(5-Methyl-1,3,4-oxadiazol-2-yl)ethoxy]-5-(4,4,5,5-tetramethyl-1,3,2-dioxaborolan-2-yl)pyridin-2-amine (172 mg, 33% purity, 161 μmol) was dissolved in 1,4-dioxane (910 μL) under argon. (3R)-1-[(Propan-2-yl)carbamoyl]-5′,6′-dihydrospiro[pyrrolidine-3,4′-pyrrolo[1,2-b]pyrazol]-2′-yl trifluoromethanesulfonate (53.3 mg, 135 μmol), potassium phosphate (810 μL, 0.50 M, 400 μmol) and XPhos Pd G2 (10.6 mg, 13.5 μmol) were added and the mixture was stirred for 1 h at 100° C. The reaction mixture was allowed to cool down, diluted with dichloromethane, washed with water and brine. The organic phase was dried over a hydrophobic filter and concentrated. The residue was purified by preparative HPLC to give 15.4 mg (100% purity, 25% yield) of the title compound.

[α]²⁰_{D}: +198.7° (c=1.00, methanol)

LC-MS (Method 1): Rₜ=0.80 min; MS (ESIpos): m/z=467 [M+H]⁺

¹H-NMR (400 MHz, DMSO-d₆): δ [ppm]=1.06 (d, 3H), 1.97 (d, 3H), 1.71 (d, 3H), 1.98-2.11 (m, 2H), 3.36-3.43 (m, 3H), 3.46-3.54 (m, 1H), 3.71-3.80 (m, 1H), 4.16 (t, 2H), 5.79-5.85 (m, 4H), 6.31 (s, 1H), 7.51 (d, 1H), 7.97 (d, 1H).

### Example 874

### (3R)-2′-{6-amino-5-[(1R)-1-(1-methyl-1H-1,2,3-triazol-5-yl)ethoxy]pyridin-3-yl}-N-(propan-2-yl)-5′,6′-dihydrospiro[pyrrolidine-3,4′-pyrrolo[1,2-b]pyrazole]-1-carboxamide

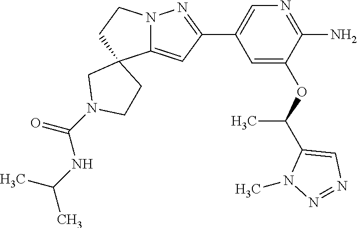 [SMILES: CC(C)NC(=O)N1CC[C@@]2(CCn3nc(-c4cnc(N)c(O[C@H](C)c5cnnn5C)c4)cc32)C1]

The compound was prepared similarly to Example 873 using (3R)-1-[(propan-2-yl)carbamoyl]-5′,6′-dihydrospiro[pyrrolidine-3,4′-pyrrolo[1,2-b]pyrazol]-2′-yl trifluoromethanesulfonate (104 mg, 98% purity, 257 μmol) and 3-[(1R)-1-(1-methyl-1H-1,2,3-triazol-5-yl)ethoxy]-5-(4,4,5,5-tetramethyl-1,3,2-dioxaborolan-2-yl)pyridin-2-amine (222 mg, 40% purity, 257 μmol) to give 47.5 mg (96% purity, 38% yield) of the title compound.

[α]²⁰_{D}: +82.7° (c=1.00, methanol)

LC-MS (Method 1): Rₜ=0.77 min; MS (ESIpos): m/z=466 [M+H]⁺

¹H-NMR (400 MHz, DMSO-d₆): δ [ppm]=1.06 (d, 3H), 1.07 (d, 3H), 1.65 (d, 3H), 1.98-2.10 (m, 2H), 3.35-3.44 (m, 3H), 3.46-3.54 (m, 1H), 3.69-3.82 (m, 1H), 4.02 (s, 3H), 4.16 (t, 2H), 5.82 (d, 1H), 5.86 (s, 2H), 5.92 (q, 1H), 6.32 (s, 1H), 7.46 (d, 1H), 7.86 (s, 1H), 7.95 (d, 1H).

### Example 875

### (3R)-2′-(6-amino-5-{[1-(1-methyl-1H-1,2,3-triazol-4-yl)ethyl]oxy}pyridin-3-yl)-N-(propan-2-yl)-5′,6′-dihydrospiro[pyrrolidine-3,4′-pyrrolo[1,2-b]pyrazole]-1-carboxamide (Diastereomer 1 and Diastereomer 2)

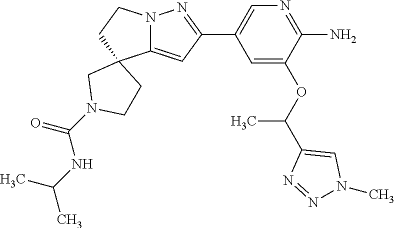 [SMILES: CC(C)NC(=O)N1CC[C@@]2(CCn3nc(-c4cnc(N)c(OC(C)c5cn(C)nn5)c4)cc32)C1]

3-{[1-(1-Methyl-1H-1,2,3-triazol-4-yl)ethyl]oxy}-5-(4,4,5,5-tetramethyl-1,3,2-dioxaborolan-2-yl)pyridin-2-amine (701 mg, 24% purity, 487 μmol) was dissolved in 1,4-dioxane (3.3 mL) under argon. (3R)-1-[(Propan-2-yl)carbamoyl]-5′,6′-dihydrospiro[pyrrolidine-3,4′-pyrrolo[1,2-b]pyrazol]-2′-yl trifluoromethanesulfonate (197 mg, 98% purity, 487 μmol), potassium phosphate (2.9 mL, 0.50 M, 1.5 mmol) and XPhos Pd G2 (19.2 mg, 24.4 μmol) were added and the mixture was stirred for 4 h at 100° C. The reaction mixture was allowed to cool down, diluted with ethyl acetate and washed with water. The organic phase was dried over a hydrophobic filter and concentrated. The residue was purified twice by preparative HPLC to give 26.6 mg (90% purity, 11% yield) of the title compound.

[α]²⁰_{D}: +48.3° (c=1.00, methanol)

LC-MS (Method 1): Rₜ=0.80 min; MS (ESIpos): m/z=466 [M+H]⁺

¹H-NMR (400 MHz, DMSO-d6) δ [ppm]: 1.053 (2.55), 1.060 (3.47), 1.069 (2.82), 1.077 (3.40), 1.233 (0.48), 1.622 (0.72), 1.629 (3.13), 1.644 (3.14), 1.664 (0.48), 2.030 (0.55), 2.043 (0.56), 2.048 (0.56), 2.336 (0.58), 2.518 (16.00), 2.523 (11.61), 2.673 (1.31), 2.678 (0.65), 3.376 (0.72), 3.395 (2.12), 3.399 (2.42), 3.566 (1.51), 3.986 (0.93), 4.007 (8.45), 4.014 (1.14), 4.019 (0.53), 4.140 (0.71), 4.158 (1.31), 4.175 (0.75), 5.654 (0.72), 5.670 (0.74), 5.727 (1.73), 5.808 (0.55), 5.828 (0.55), 6.311 (2.28), 7.456 (1.19), 7.460 (1.27), 7.905 (1.04), 7.906 (1.25), 7.909 (1.27), 7.911 (1.20), 8.116 (2.52).

### Example 876

### (5S)-5-{[(3R)-2′-{6-amino-5-[(1R)-1-phenylethoxy]pyridin-3-yl}-5′,6′-dihydrospiro[pyrrolidine-3,4′-pyrrolo[1,2-b]pyrazol]-1-yl]methyl}pyrrolidin-2-one

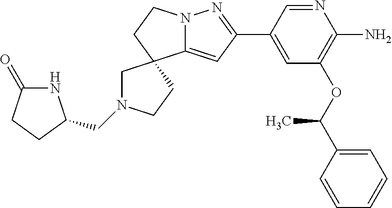 [SMILES: C[C@@H](Oc1cc(-c2cc3n(n2)CC[C@@]32CCN(C[C@@H]3CCC(=O)N3)C2)cnc1N)c1ccccc1]

5-[(3R)-5′,6′-Dihydrospiro[pyrrolidine-3,4′-pyrrolo[1,2-b]pyrazol]-2′-yl]-3-[(1R)-1-phenylethoxy]pyridin-2-amine (20.0 mg, 100% purity, 53.3 μmol) and (5S)-5-(bromomethyl)pyrrolidin-2-one (11.4 mg, 63.9 μmol) were dissolved in THF (1.2 mL) under argon. Potassium carbonate (29.4 mg, 213 μmol) was added and the mixture was stirred for 2 h at rt. Sodium iodide (7.98 mg, 53.3 μmol) was added and the mixture was stirred over night at 70° C. The reaction mixture was allowed to cool down, concentrated and purified by preparative HPLC to give 4.10 mg (95% purity, 15% yield) of the title compound.

LC-MS (Method 1): Rₜ=1.00 min; MS (ESIpos): m/z=474 [M+H]⁺

¹H-NMR (400 MHz, DMSO-d6) δ [ppm]: 1.233 (0.48), 1.561 (11.74), 1.577 (11.65), 1.686 (0.62), 1.703 (1.54), 1.720 (0.90), 1.730 (0.87), 1.734 (0.89), 1.914 (0.42), 1.935 (0.67), 1.946 (1.16), 1.963 (1.47), 1.983 (1.58), 1.999 (1.33), 2.005 (1.41), 2.019 (1.40), 2.030 (0.75), 2.037 (0.63), 2.052 (0.78), 2.061 (1.12), 2.075 (2.48), 2.088 (1.66), 2.092 (1.47), 2.097 (2.02), 2.104 (3.49), 2.111 (2.48), 2.122 (2.34), 2.132 (1.54), 2.140 (2.02), 2.165 (1.90), 2.178 (0.57), 2.190 (0.78), 2.318 (1.04), 2.323 (2.32), 2.327 (3.25), 2.331 (2.32), 2.336 (1.05), 2.365 (1.46), 2.381 (1.62), 2.395 (2.21), 2.411 (2.46), 2.428 (1.37), 2.440 (1.60), 2.460 (2.57), 2.518 (16.00), 2.523 (10.43), 2.565 (3.85), 2.579 (1.20), 2.593 (1.47), 2.598 (1.41), 2.611 (1.84), 2.624 (1.31), 2.630 (1.46), 2.652 (1.88), 2.660 (1.57), 2.665 (3.15), 2.669 (4.92), 2.673 (3.58), 2.690 (0.88), 2.728 (3.54), 2.750 (3.59), 2.765 (1.27), 2.772 (1.62), 2.787 (1.51), 2.808 (0.60), 3.601 (0.95), 3.616 (1.53), 3.632 (1.21), 3.995 (0.41), 4.014 (0.66), 4.021 (1.56), 4.038 (3.29), 4.052 (2.19), 4.058 (2.78), 4.072 (1.64), 4.085 (0.58), 4.098 (0.40), 5.556 (0.73), 5.572 (2.57), 5.588 (2.63), 5.604 (0.78), 5.814 (7.69), 6.148 (0.46), 6.244 (15.09), 7.226 (1.71), 7.234 (5.55), 7.238 (6.18), 7.244 (4.26), 7.249 (1.41), 7.259 (1.68), 7.262 (2.77), 7.265 (1.54), 7.319 (4.19), 7.338 (7.85), 7.352 (1.42), 7.357 (3.83), 7.454 (6.63), 7.472 (5.27), 7.476 (3.92), 7.641 (4.43), 7.845 (7.25), 7.850 (7.09), 8.553 (1.22).

### Example 877

### (5R)-5-{[(3R)-2′-{6-amino-5-[(1R)-1-phenylethoxy]pyridin-3-yl}-5′,6′-dihydrospiro[pyrrolidine-3,4′-pyrrolo[1,2-b]pyrazol]-1-yl]methyl}pyrrolidin-2-one

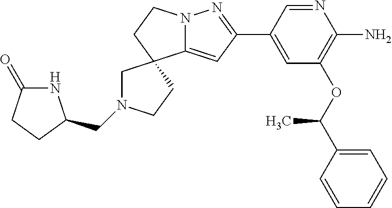 [SMILES: C[C@@H](Oc1cc(-c2cc3n(n2)CC[C@@]32CCN(C[C@H]3CCC(=O)N3)C2)cnc1N)c1ccccc1]

5-[(3R)-5′,6′-Dihydrospiro[pyrrolidine-3,4′-pyrrolo[1,2-b]pyrazol]-2′-yl]-3-[(1R)-1-phenylethoxy]pyridin-2-amine (20.0 mg, 100% purity, 53.3 μmol) and (5R)-5-(bromomethyl)pyrrolidin-2-one (11.4 mg, 63.9 μmol) were dissolved in THF (1.2 mL). Potassium carbonate (29.4 mg, 213 μmol) and sodium iodide (7.98 mg, 53.3 μmol) were added and the mixture was stirred over night at 70° C. The reaction mixture was allowed to cool down and concentrated. The residue was purified by preparative HPLC to give 10 mg (95% purity, 38% yield) of the title compound.

LC-MS (Method 1): Rₜ=1.00 min; MS (ESIpos): m/z=474 [M+H]⁺

¹H-NMR (400 MHz, DMSO-d6) δ [ppm]: 1.232 (0.48), 1.346 (0.42), 1.562 (12.64), 1.578 (12.56), 1.706 (0.99), 1.712 (1.21), 1.720 (1.17), 1.734 (1.32), 1.748 (0.74), 1.938 (0.61), 1.950 (1.32), 1.970 (1.97), 1.988 (2.07), 1.995 (1.70), 2.010 (1.45), 2.021 (0.57), 2.027 (0.58), 2.041 (0.51), 2.067 (1.05), 2.083 (2.33), 2.099 (1.18), 2.110 (5.78), 2.126 (2.49), 2.131 (2.75), 2.137 (2.61), 2.151 (0.57), 2.162 (1.97), 2.185 (0.76), 2.387 (1.50), 2.402 (1.69), 2.417 (2.98), 2.433 (3.25), 2.450 (2.12), 2.469 (4.15), 2.518 (15.29), 2.523 (10.38), 2.572 (3.74), 2.580 (1.63), 2.594 (4.39), 2.620 (1.96), 2.636 (1.71), 2.659 (1.58), 2.740 (3.79), 2.763 (3.06), 2.784 (0.90), 2.805 (1.61), 2.821 (1.52), 2.841 (0.65), 3.608 (1.16), 3.622 (1.75), 3.638 (1.26), 4.033 (1.99), 4.039 (1.93), 4.051 (3.54), 4.067 (1.91), 4.072 (1.97), 5.557 (0.77), 5.573 (2.64), 5.589 (2.69), 5.604 (0.77), 5.813 (7.96), 6.238 (16.00), 7.226 (1.95), 7.232 (5.61), 7.236 (5.75), 7.244 (4.24), 7.249 (1.45), 7.259 (1.69), 7.262 (3.06), 7.265 (1.69), 7.318 (4.40), 7.338 (8.24), 7.351 (1.48), 7.356 (4.00), 7.454 (6.88), 7.472 (5.47), 7.476 (4.00), 7.620 (4.57), 7.842 (8.34), 7.847 (8.18), 8.552 (1.64).

### Example 878

### (3R)-2′-(6-amino-5-{1-[4-(methylsulfonyl)phenyl]ethoxy}pyridin-3-yl)-N-isopropyl-5′,6′-dihydro-1H-spiro[pyrrolidine-3,4′-pyrrolo[1,2-b]pyrazole]-1-carboxamide (Diastereomer 1 and Diastereomer 2)

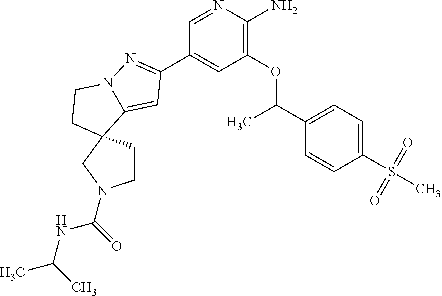 [SMILES: CC(C)NC(=O)N1CC[C@@]2(CCn3nc(-c4cnc(N)c(OC(C)c5ccc(S(C)(=O)=O)cc5)c4)cc32)C1]

3-({1-[4-(Methanesulfonyl)phenyl]ethyl}oxy)-5-(4,4,5,5-tetramethyl-1,3,2-dioxaborolan-2-yl)pyridin-2-amine (111 mg, 265 μmol) was dissolved in degassed 1,4-dioxane (3 mL) under argon. (3R)-1-[(Propan-2-yl)carbamoyl]-5′,6′-dihydrospiro[pyrrolidine-3,4′-pyrrolo[1,2-b]pyrazol]-2′-yl trifluoromethanesulfonate (110 mg, 279 μmol), potassium phosphate (1.6 mL, 0.50 M, 800 μmol) and XPhos Pd G2 (10.4 mg, 13.3 μmol) were added and the mixture was stirred for 1 h at 100° C. The cooled down reaction mixture was diluted with water and extracted with ethyl acetate. The combined organic layers were washed with brine, dried over sodium sulfate, filtered and concentrated. The residue was purified by preparative HPLC to give 5.00 mg (95% purity, 3% yield) of the title compound.

LC-MS (Method 1): Rₜ=0.89 min; MS (ESIpos): m/z=540 [M+H]⁺

¹H-NMR (400 MHz, DMSO-d6) δ [ppm]: 1.045 (5.15), 1.052 (5.98), 1.056 (6.81), 1.060 (8.36), 1.068 (6.09), 1.072 (6.32), 1.076 (5.39), 1.572 (6.44), 1.588 (6.43), 1.606 (0.41), 1.987 (0.70), 1.998 (1.00), 2.016 (1.51), 2.023 (1.32), 2.035 (0.65), 2.041 (0.52), 2.464 (1.72), 2.518 (1.71), 2.523 (1.17), 3.198 (16.00), 3.371 (5.80), 3.379 (5.59), 3.398 (0.99), 3.457 (0.54), 3.473 (0.93), 3.486 (0.68), 3.498 (0.67), 3.725 (0.51), 3.730 (0.55), 3.745 (0.84), 3.761 (0.86), 3.777 (0.53), 3.782 (0.46), 4.106 (1.85), 4.124 (3.25), 4.141 (1.76), 5.748 (0.45), 5.765 (1.48), 5.781 (1.49), 5.800 (1.36), 5.805 (1.17), 5.819 (1.13), 5.825 (1.06), 5.940 (4.51), 6.257 (5.13), 6.260 (4.83), 7.275 (3.19), 7.279 (3.20), 7.754 (3.46), 7.774 (4.40), 7.864 (2.86), 7.866 (3.48), 7.868 (3.23), 7.871 (2.88), 7.895 (4.03), 7.898 (4.50), 7.916 (3.22), 7.919 (2.99).

### Example 879

### (3R)-2′-{6-amino-5-[(1R)-1-(2-methyl-1,3-thiazol-4-yl)ethoxy]pyridin-3-yl}-N-(propan-2-yl)-5′,6′-dihydrospiro[pyrrolidine-3,4′-pyrrolo[1,2-b]pyrazole]-1-carboxamide

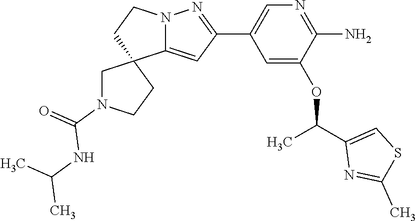 [SMILES: Cc1nc([C@@H](C)Oc2cc(-c3cc4n(n3)CC[C@@]43CCN(C(=O)NC(C)C)C3)cnc2N)cs1]

The compound was prepared similarly to Example 878 using (3R)-1-[(propan-2-yl)carbamoyl]-5′,6′-dihydrospiro[pyrrolidine-3,4′-pyrrolo[1,2-b]pyrazol]-2′-yl trifluoromethanesulfonate (83.3 mg, 98% purity, 206 μmol) and 3-[(1R)-1-(2-methyl-1,3-thiazol-4-yl)ethoxy]-5-(4,4,5,5-tetramethyl-1,3,2-dioxaborolan-2-yl)pyridin-2-amine (186 mg, 40% purity, 206 μmol) to give 26.9 mg (96% purity, 26% yield) of the title compound.

[α]²⁰_{D}: +119.17° (c=1.00, methanol)

LC-MS (Method 1): Rₜ=0.90 min; MS (ESIneg): m/z=480 [M−H]⁻

¹H-NMR (400 MHz, DMSO-d6) δ [ppm]: 1.052 (6.31), 1.060 (6.73), 1.068 (6.70), 1.076 (6.30), 1.605 (5.51), 1.621 (5.52), 1.893 (3.12), 2.012 (0.68), 2.026 (1.08), 2.039 (1.41), 2.057 (0.60), 2.323 (0.73), 2.327 (1.00), 2.332 (0.75), 2.518 (7.20), 2.523 (4.32), 2.647 (16.00), 2.660 (0.55), 2.665 (0.84), 2.669 (1.08), 2.673 (0.77), 3.375 (1.22), 3.392 (5.95), 3.419 (0.51), 3.468 (0.43), 3.486 (0.71), 3.494 (0.47), 3.501 (0.53), 3.512 (0.45), 3.730 (0.50), 3.746 (0.73), 3.765 (0.75), 3.782 (0.49), 4.130 (1.37), 4.147 (2.49), 4.164 (1.33), 5.568 (1.24), 5.584 (1.23), 5.784 (3.50), 5.806 (1.40), 5.825 (1.26), 6.297 (5.37), 7.375 (2.27), 7.380 (2.27), 7.467 (4.54), 7.915 (3.05), 7.919 (3.00).

### Example 880

### (3R)-2′-[6-amino-5-({1-[3-(methanesulfonyl)phenyl]ethyl}oxy)pyridin-3-yl]-N-(propan-2-yl)-5′,6′-dihydrospiro[pyrrolidine-3,4′-pyrrolo[1,2-b]pyrazole]-1-carboxamide (Diastereomer 1 and Diastereomer 2)

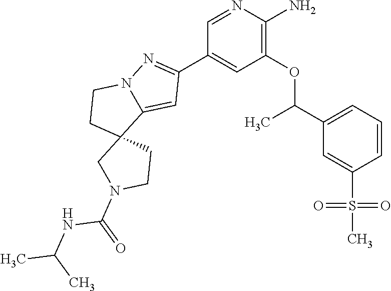 [SMILES: CC(C)NC(=O)N1CC[C@@]2(CCn3nc(-c4cnc(N)c(OC(C)c5cccc(S(C)(=O)=O)c5)c4)cc32)C1]

The compound was prepared similarly to Example 878 using 3-({1-[3-(methanesulfonyl)phenyl]ethyl}oxy)-5-(4,4,5,5-tetramethyl-1,3,2-dioxaborolan-2-yl)pyridin-2-amine (555 mg, 1.33 mmol) and (3R)-1-[(propan-2-yl)carbamoyl]-5′,6′-dihydrospiro[pyrrolidine-3,4′-pyrrolo[1,2-b]pyrazol]-2′-yl trifluoromethanesulfonate (552 mg, 1.39 mmol) to give 136 mg (98% purity, 19% yield) of the title compound.

LC-MS (Method 1): Rₜ=0.90 min; MS (ESIpos): m/z=540 [M+H]⁺

¹H-NMR (400 MHz, DMSO-d6) δ [ppm]: 1.047 (6.20), 1.052 (7.25), 1.060 (9.84), 1.069 (7.43), 1.075 (8.10), 1.595 (7.51), 1.610 (7.48), 2.008 (1.74), 2.017 (2.28), 2.035 (1.03), 2.465 (2.09), 2.518 (2.12), 2.523 (1.36), 3.210 (16.00), 3.212 (15.12), 3.371 (6.46), 3.376 (5.52), 3.388 (1.98), 3.403 (1.19), 3.457 (0.69), 3.474 (1.18), 3.488 (0.86), 3.499 (0.79), 3.514 (0.43), 3.730 (0.70), 3.747 (1.11), 3.763 (1.08), 3.779 (0.64), 4.098 (2.04), 4.115 (3.81), 4.133 (2.03), 5.738 (0.49), 5.754 (1.69), 5.770 (1.72), 5.786 (0.68), 5.803 (1.56), 5.822 (1.47), 5.919 (4.88), 6.254 (5.21), 6.259 (4.75), 7.296 (3.42), 7.300 (3.51), 7.619 (1.59), 7.638 (3.51), 7.657 (2.16), 7.814 (2.22), 7.818 (1.91), 7.835 (1.94), 7.838 (1.94), 7.844 (2.44), 7.864 (6.10), 7.868 (5.36), 8.094 (2.66), 8.100 (2.51).

### Example 881

### (3R)-2′-(6-amino-5-{[1-(4-methyl-1,3-thiazol-2-yl)ethyl]oxy}pyridin-3-yl)-N-ethyl-5′,6′-dihydrospiro[pyrrolidine-3,4′-pyrrolo[1,2-b]pyrazole]-1-carboxamide (Diastereomer 1 and Diastereomer 2)

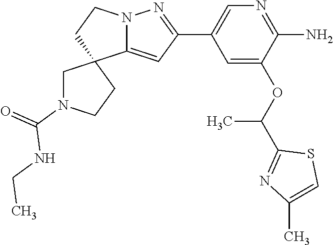 [SMILES: CCNC(=O)N1CC[C@@]2(CCn3nc(-c4cnc(N)c(OC(C)c5nc(C)cs5)c4)cc32)C1]

3-{[1-(4-Methyl-1,3-thiazol-2-yl)ethyl]oxy}-5-(4,4,5,5-tetramethyl-1,3,2-dioxaborolan-2-yl)pyridin-2-amine (198 mg, 44% purity, 241 μmol) was dissolved in 1,4-dioxane (1.4 mL) under argon. (3R)-1-(Ethylcarbamoyl)-5′,6′-dihydrospiro[pyrrolidine-3,4′-pyrrolo[1,2-b]pyrazol]-2′-yl trifluoromethanesulfonate (76.8 mg, 201 μmol), potassium phosphate (1.2 mL, 0.50 M, 600 μmol) and XPhos Pd G2 (15.8 mg, 20.1 μmol) were added and the mixture was stirred for 1 h at 100° C. The reaction mixture was allowed to cool down, diluted with dichloromethane and washed with water and brine. The organic phase was dried over a hydrophobic filter and concentrated. The residue was purified by preparative HPLC to give 29.0 mg (93% purity, 29% yield) of the title compound.

LC-MS (Method 1): Rₜ=0.89 min; MS (ESIpos): m/z=468 [M+H]⁺

¹H-NMR (400 MHz, DMSO-d6) δ [ppm]: 0.999 (6.45), 1.017 (15.13), 1.035 (6.76), 1.677 (9.47), 1.693 (9.38), 2.030 (1.64), 2.037 (2.04), 2.055 (0.92), 2.332 (1.18), 2.337 (0.73), 2.347 (10.27), 2.350 (16.00), 2.353 (9.88), 2.518 (7.89), 2.523 (4.83), 2.673 (1.05), 2.679 (0.48), 3.016 (0.78), 3.034 (2.48), 3.048 (2.80), 3.051 (2.70), 3.065 (2.48), 3.083 (0.72), 3.357 (0.83), 3.383 (8.50), 3.399 (1.81), 3.418 (0.69), 3.447 (0.65), 3.465 (1.20), 3.473 (0.67), 3.480 (0.82), 3.490 (0.70), 4.125 (2.32), 4.143 (3.75), 4.160 (2.23), 5.793 (0.77), 5.808 (3.07), 5.821 (6.16), 5.840 (0.91), 6.133 (1.03), 6.147 (2.02), 6.160 (1.00), 6.275 (10.12), 7.233 (3.87), 7.235 (4.63), 7.237 (3.72), 7.405 (3.59), 7.938 (4.78), 7.943 (4.59).

### Example 882

### (3R)-2′-(6-amino-5-{[1-(4,5-dimethyl-1,3-thiazol-2-yl)ethyl]oxy}pyridin-3-yl)-N-ethyl-5′,6′-dihydrospiro[pyrrolidine-3,4′-pyrrolo[1,2-b]pyrazole]-1-carboxamide (Diastereomer 1 and Diastereomer 2)

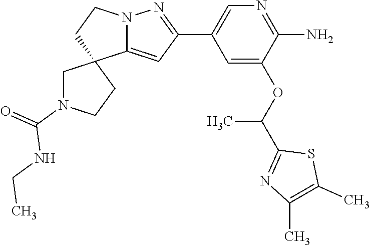 [SMILES: CCNC(=O)N1CC[C@@]2(CCn3nc(-c4cnc(N)c(OC(C)c5nc(C)c(C)s5)c4)cc32)C1]

3-{[1-(4,5-Dimethyl-1,3-thiazol-2-yl)ethyl]oxy}-5-(4,4,5,5-tetramethyl-1,3,2-dioxaborolan-2-yl)pyridin-2-amine (350 mg, 39% purity, 364 μmol) was dissolved in 1,4-dioxane (2.1 mL) under argon. (3R)-1-(Ethylcarbamoyl)-5′,6′-dihydrospiro[pyrrolidine-3,4′-pyrrolo[1,2-b]pyrazol]-2′-yl trifluoromethanesulfonate (116 mg, 303 μmol), potassium phosphate (1.8 mL, 0.50 M, 910 μmol) and XPhos Pd G2 (23.8 mg, 30.3 μmol) were added and the mixture was stirred for 1.5 h at 100° C. The reaction mixture was allowed to cool down, diluted with dichloromethane and washed with water and brine. The organic phase was dried over a hydrophobic filter and concentrated. The residue was purified by preparative HPLC to give 85.0 mg (100% purity, 58% yield) of the title compound.

LC-MS (Method 2): Rₜ=0.79 min; MS (ESIpos): m/z=482 [M+H]⁺

¹H-NMR (400 MHz, DMSO-d6) δ [ppm]: 1.000 (6.67), 1.017 (16.00), 1.035 (7.20), 1.651 (9.58), 1.667 (9.62), 2.030 (1.56), 2.037 (1.88), 2.055 (0.86), 2.226 (9.47), 2.228 (12.46), 2.231 (9.57), 2.278 (14.06), 2.332 (0.69), 2.518 (5.58), 2.523 (2.79), 2.673 (0.69), 3.017 (0.77), 3.034 (2.46), 3.048 (2.70), 3.052 (2.64), 3.066 (2.42), 3.084 (0.72), 3.358 (0.84), 3.384 (8.33), 3.401 (1.78), 3.419 (0.71), 3.448 (0.68), 3.466 (1.23), 3.473 (0.67), 3.480 (0.83), 3.491 (0.71), 4.126 (2.32), 4.144 (3.68), 4.161 (2.25), 5.704 (0.57), 5.720 (2.10), 5.736 (2.11), 5.752 (0.60), 5.797 (5.39), 6.134 (1.02), 6.148 (2.08), 6.162 (1.02), 6.268 (11.18), 7.396 (3.76), 7.925 (5.27), 7.930 (5.20), 8.138 (0.71).

### Example 883

### (3R)-2′-(6-amino-5-{[1-(5-methyl-1,3-thiazol-2-yl)ethyl]oxy}pyridin-3-yl)-N-ethyl-5′,6′-dihydrospiro[pyrrolidine-3,4′-pyrrolo[1,2-b]pyrazole]-1-carboxamide (Diastereomer 1 and Diastereomer 2)

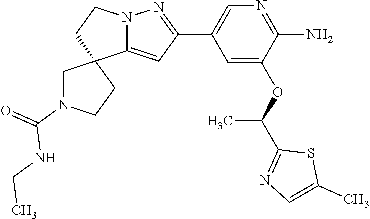 [SMILES: CCNC(=O)N1CC[C@@]2(CCn3nc(-c4cnc(N)c(O[C@H](C)c5ncc(C)s5)c4)cc32)C1]

The compound was prepared similarly to Example 882 using (3R)-1-(ethylcarbamoyl)-5′,6′-dihydrospiro[pyrrolidine-3,4′-pyrrolo[1,2-b]pyrazol]-2′-yl trifluoromethanesulfonate (111 mg, 290 μmol) and 3-{[1-(5-methyl-1,3-thiazol-2-yl)ethyl]oxy}-5-(4,4,5,5-tetramethyl-1,3,2-dioxaborolan-2-yl)pyridin-2-amine (331 mg, 38% purity, 348 μmol) to give 37.0 mg (100% purity, 27% yield) of the title compound.

LC-MS (Method 1): Rₜ=0.89 min; MS (ESIpos): m/z=468 [M+H]⁺

¹H-NMR (400 MHz, DMSO-d6) δ [ppm]: 0.998 (5.08), 1.016 (11.57), 1.018 (10.49), 1.034 (5.22), 1.036 (4.88), 1.672 (10.46), 1.688 (10.59), 2.017 (1.09), 2.028 (1.63), 2.035 (1.70), 2.043 (1.79), 2.054 (0.78), 2.061 (0.65), 2.077 (0.45), 2.392 (16.00), 2.480 (3.43), 2.518 (4.63), 2.523 (2.83), 3.016 (0.64), 3.033 (2.16), 3.050 (2.84), 3.065 (2.03), 3.067 (2.03), 3.083 (0.63), 3.351 (0.87), 3.370 (1.48), 3.382 (5.45), 3.385 (6.25), 3.394 (2.12), 3.413 (0.77), 3.450 (0.69), 3.468 (1.18), 3.475 (0.71), 3.483 (0.86), 3.494 (0.69), 3.509 (0.41), 4.126 (2.40), 4.144 (3.97), 4.161 (2.36), 5.755 (0.69), 5.772 (2.53), 5.787 (2.70), 5.812 (5.77), 6.134 (1.07), 6.148 (2.21), 6.162 (1.08), 6.275 (7.83), 7.401 (3.96), 7.438 (3.91), 7.440 (4.53), 7.443 (4.25), 7.923 (4.37), 7.927 (3.98), 8.133 (0.73).

### Example 884

### (3R)-2′-{6-amino-5-[(1R)-1-(pyridin-4-yl)ethoxy]pyridin-3-yl}-N-(propan-2-yl)-5′,6′-dihydrospiro[pyrrolidine-3,4′-pyrrolo[1,2-b]pyrazole]-1-carboxamide

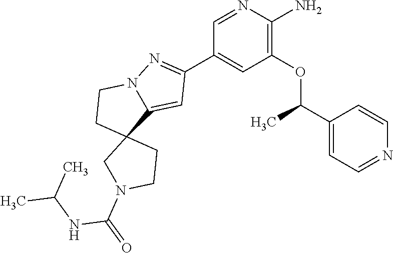 [SMILES: CC(C)NC(=O)N1CC[C@@]2(CCn3nc(-c4cnc(N)c(O[C@H](C)c5ccncc5)c4)cc32)C1]

5-[(3R)-5′,6′-Dihydrospiro[pyrrolidine-3,4′-pyrrolo[1,2-b]pyrazol]-2′-yl]-3-[(1R)-1-(pyridin-4-yl)ethoxy]pyridin-2-amine hydrogen chloride (1/1) (40.0 mg, 58% purity, 56.2 μmol, Intermediate 532) was suspended in dichloromethane (0.5 mL). N,N-Diisopropylethylamine (98 μL, 560 μmol) was added and the mixture was cooled to 0° C. 2-Isocyanatopropane (6.6 μL, 67 μmol) was added and the mixture was allowed to warm up to rt and stirred overnight. The reaction mixture was concentrated. The residue was purified by preparative TLC (dichloromethane/methanol 9:1 gradient) to give 10.0 mg (93% purity, 36% yield) of the title compound.

LC-MS (Method 1): Rₜ=0.86 min; MS (ESIpos): m/z=462 [M+H]⁺

¹H-NMR (400 MHz, DMSO-d6) δ [ppm]: 0.834 (0.41), 0.852 (1.02), 0.868 (1.31), 0.886 (1.07), 0.906 (1.88), 0.924 (0.92), 1.045 (14.05), 1.057 (16.00), 1.062 (15.93), 1.073 (14.43), 1.233 (4.79), 1.256 (2.61), 1.332 (0.42), 1.353 (0.49), 1.393 (0.43), 1.410 (0.43), 1.555 (12.06), 1.571 (11.99), 1.907 (0.60), 1.995 (1.56), 2.008 (2.34), 2.022 (3.12), 2.040 (1.32), 2.052 (0.63), 2.112 (0.59), 2.466 (4.45), 2.518 (12.12), 2.523 (8.41), 2.678 (0.97), 3.371 (12.06), 3.388 (2.45), 3.406 (0.96), 3.454 (0.88), 3.471 (1.55), 3.479 (1.01), 3.486 (1.14), 3.493 (0.93), 3.511 (0.55), 3.725 (1.12), 3.742 (1.64), 3.761 (1.68), 3.777 (1.12), 3.940 (0.46), 4.104 (3.04), 4.122 (5.48), 4.139 (2.94), 4.228 (0.44), 4.234 (0.48), 4.242 (0.46), 4.248 (0.42), 5.632 (0.73), 5.647 (2.51), 5.663 (2.52), 5.679 (0.71), 5.760 (11.88), 5.800 (2.74), 5.819 (2.68), 5.929 (7.41), 6.247 (12.76), 7.236 (5.10), 7.240 (4.94), 7.476 (7.88), 7.479 (4.69), 7.487 (4.91), 7.491 (7.94), 7.875 (7.51), 7.879 (7.28), 8.089 (3.12), 8.533 (10.08), 8.537 (5.64), 8.544 (5.67), 8.548 (9.01).

### Example 885

### (3R)-2′-{6-amino-5-[(1R)-1-(pyridin-4-yl)ethoxy]pyridin-3-yl}-N-cyclobutyl-5′,6′-dihydrospiro[pyrrolidine-3,4′-pyrrolo[1,2-b]pyrazole]-1-carboxamide

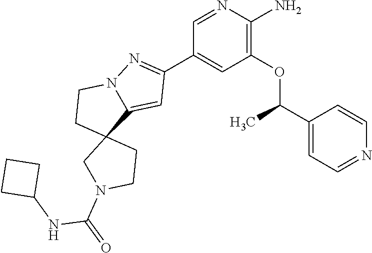 [SMILES: C[C@@H](Oc1cc(-c2cc3n(n2)CC[C@@]32CCN(C(=O)NC3CCC3)C2)cnc1N)c1ccncc1]

The compound was prepared similarly to Example 884 using 5-[(3R)-5′,6′-dihydrospiro[pyrrolidine-3,4′-pyrrolo[1,2-b]pyrazol]-2′-yl]-3-[(1R)-1-(pyridin-4-yl)ethoxy]pyridin-2-amine hydrogen chloride (1/1) (40.0 mg, 58% purity, 56.2 μmol, Intermediate 532) and isocyanatocyclobutane (6.55 mg, 67.4 μmol) to give 7.70 mg (100% purity, 29% yield) of the title compound.

LC-MS (Method 1): Rₜ=0.87 min; MS (ESIpos): m/z=474 [M+H]⁺

¹H-NMR (400 MHz, DMSO-d6) δ [ppm]: 0.833 (0.65), 0.851 (1.80), 0.868 (3.50), 0.887 (2.94), 0.906 (5.32), 0.924 (2.45), 1.232 (4.06), 1.256 (1.11), 1.279 (1.47), 1.289 (1.65), 1.296 (1.76), 1.317 (1.34), 1.339 (0.88), 1.353 (1.09), 1.374 (0.84), 1.392 (1.08), 1.411 (1.20), 1.431 (0.83), 1.450 (0.40), 1.484 (0.78), 1.503 (1.43), 1.510 (1.20), 1.522 (1.31), 1.529 (2.73), 1.553 (14.67), 1.570 (13.48), 1.684 (0.49), 1.700 (0.57), 1.715 (0.43), 1.885 (1.20), 1.912 (2.13), 1.933 (2.20), 1.960 (1.32), 1.995 (1.71), 2.009 (2.47), 2.020 (3.15), 2.038 (1.41), 2.051 (0.82), 2.069 (1.31), 2.078 (2.06), 2.085 (2.63), 2.095 (2.09), 2.104 (2.33), 2.112 (1.84), 2.123 (0.85), 2.318 (1.24), 2.463 (4.76), 2.518 (16.00), 2.523 (11.07), 3.367 (9.94), 3.383 (1.54), 3.391 (2.67), 3.409 (1.01), 3.446 (0.95), 3.464 (1.74), 3.472 (1.08), 3.479 (1.26), 3.490 (1.05), 3.504 (1.06), 4.079 (0.91), 4.102 (4.48), 4.120 (6.98), 4.137 (3.68), 4.228 (1.34), 4.234 (1.43), 4.242 (1.43), 4.248 (1.32), 5.635 (0.76), 5.651 (2.67), 5.667 (2.70), 5.682 (0.77), 5.760 (14.90), 5.927 (8.11), 6.251 (14.59), 6.293 (2.86), 6.312 (2.84), 7.240 (5.32), 7.243 (5.40), 7.476 (8.40), 7.479 (5.10), 7.487 (5.24), 7.491 (8.72), 7.879 (7.75), 7.883 (7.62), 8.089 (9.47), 8.533 (10.46), 8.537 (6.00), 8.544 (6.00), 8.548 (10.36).

### Example 886

### 6-(1-{(3′R)-2-[6-amino-5-(trifluoromethyl)pyridin-3-yl]-6,7-dihydrospiro[pyrazolo[5,1-c][1,4]oxazine-4,3′-pyrrolidin]-1′-yl}ethyl)pyridin-2-ol (Diastereomer 1 and Diastereomer 2)

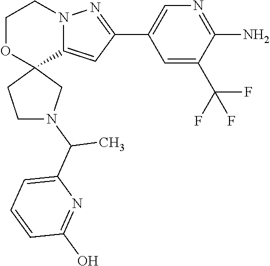 [SMILES: CC(c1cccc(O)n1)N1CC[C@]2(C1)OCCn1nc(-c3cnc(N)c(C(F)(F)F)c3)cc12]

5-{(3′R)-1′-[1-(6-Methoxypyridin-2-yl)ethyl]-6,7-dihydrospiro[pyrazolo[5,1-c][1,4]oxazine-4,3′-pyrrolidin]-2-yl}-3-(trifluoromethyl)pyridin-2-amine (27.3 mg, 57.5 μmol, Example 855) was dissolved in acetonitrile (0.4 mL). Sodium iodide (43.1 mg, 288 μmol) was added and chlorotrimethylsilane (37 μL, 290 μmol) was added dropwise. The mixture was stirred over night at room temperature. Sodium iodide (43.1 mg, 288 μmol) and chlorotrimethylsilane (37 μL, 290 μmol) were added again and stirred 2 h at 50° C. and 3 h at 60° C. The reaction mixture was concentrated. The residue was diluted with ethyl acetate and washed with brine. The organic phase was dried over sodium sulfate, filtered and concentrated. The crude product was purified by preparative TLC (dichloromethane/methanol 9:1 gradient) to give 17.1 mg (95% purity, 61% yield) of the title compound.

LC-MS (Method 1): Rₜ=0.93 min; MS (ESIpos): m/z=462 [M+H]⁺

¹H-NMR (400 MHz, CHLOROFORM-d) δ [ppm]: 1.260 (1.03), 1.406 (3.48), 1.417 (5.65), 1.422 (4.32), 1.434 (5.10), 1.512 (0.45), 1.517 (0.49), 1.535 (0.55), 1.634 (1.40), 2.101 (0.60), 2.224 (0.80), 2.243 (0.87), 2.258 (1.31), 2.277 (1.14), 2.296 (0.41), 2.356 (0.60), 2.372 (0.85), 2.387 (0.79), 2.403 (0.55), 2.806 (0.40), 2.831 (0.45), 2.844 (0.44), 2.854 (0.55), 2.867 (0.56), 2.913 (0.47), 2.931 (1.02), 2.950 (1.89), 2.976 (1.92), 3.004 (0.85), 3.016 (1.56), 3.096 (1.15), 3.123 (0.82), 3.319 (0.64), 3.455 (0.79), 3.471 (1.29), 3.487 (0.93), 4.078 (0.56), 4.093 (1.14), 4.096 (1.14), 4.110 (2.30), 4.119 (2.29), 4.130 (1.44), 4.152 (0.45), 4.159 (0.47), 4.176 (1.48), 4.179 (1.47), 4.187 (3.50), 4.198 (3.39), 4.209 (1.01), 5.057 (3.91), 5.310 (16.00), 6.073 (1.40), 6.089 (1.57), 6.339 (3.29), 6.349 (1.99), 6.448 (1.44), 6.464 (1.22), 6.470 (1.70), 6.486 (1.18), 7.007 (0.45), 7.332 (1.33), 7.338 (0.76), 7.349 (1.40), 7.355 (1.88), 7.361 (0.83), 7.372 (1.28), 7.378 (0.67), 7.529 (0.47), 8.121 (1.89), 8.127 (3.02), 8.133 (1.72), 8.629 (1.73), 8.635 (2.08).

### Example 887

### 6-(1-{(3′R)-2-[6-amino-5-(trifluoromethyl)pyridin-3-yl]-6,7-dihydrospiro[pyrazolo[5,1-c][1,4]oxazine-4,3′-pyrrolidin]-1′-yl}ethyl)pyridin-2-ol (Diastereomer 1)

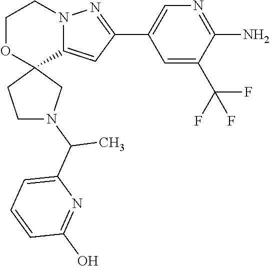 [SMILES: CC(c1cccc(O)n1)N1CC[C@]2(C1)OCCn1nc(-c3cnc(N)c(C(F)(F)F)c3)cc12]

The compound 6-(1-{(3′R)-2-[6-amino-5-(trifluoromethyl)pyridin-3-yl]-6,7-dihydrospiro[pyrazolo[5,1-c][1,4]oxazine-4,3′-pyrrolidin]-1′-yl}ethyl)pyridin-2-ol (diastereomer 1 and diastereomer 2, Example 886) was separated into diastereomers by preparative chiral HPLC to give 3.50 mg (90% purity, 21% yield) of the title compound (diastereomer 1 Rₜ=11.25-13.73 min).

Preparative Chiral HPLC Method:

Instrument: PrepCon Labomatic HPLC-2; Column: YMC Cellulose SB 5μ, 250×50; eluent A: hexane+0.1 vol % diethylamine; eluent B: 2-propanol; isocratic: 30% B; flow: 50 mL/min; temperature: 25° C.; UV: 280 nm

Analytical Chiral HPLC Method:

Instrument: Thermo Fisher UltiMate 3000; Column: YMC Cellulose SB 3μ, 100×4.6; eluent A: hexane+0.1 vol % diethylamine; eluent B: 2-propanol; isocratic: 30% B; flow: 1.4 mL/min; temperature: 25° C.; UV: 280 nm

Analytical chiral HPLC Rₜ=2.21 min

[α]²⁰_{D}: +32.6° (c=1.00, methanol)

LC-MS (Method 1): Rₜ=0.93 min; MS (ESIpos): m/z=462 [M+H]⁺

1H-NMR (400 MHz, CHLOROFORM-d) δ [ppm]: 0.764 (0.62), 0.776 (0.74), 0.783 (1.10), 0.795 (2.65), 0.800 (1.24), 0.810 (2.88), 0.814 (4.85), 0.828 (2.56), 0.833 (2.53), 0.838 (1.26), 0.845 (2.12), 0.856 (1.86), 0.859 (0.74), 0.864 (0.92), 0.874 (0.96), 0.883 (0.47), 0.923 (0.41), 0.971 (0.48), 1.066 (0.74), 1.076 (3.31), 1.095 (0.78), 1.100 (3.26), 1.114 (0.52), 1.116 (0.53), 1.120 (0.45), 1.126 (0.63), 1.129 (0.67), 1.135 (1.93), 1.142 (1.69), 1.151 (8.86), 1.168 (1.93), 1.184 (10.20), 1.215 (1.83), 1.230 (1.37), 1.253 (1.32), 1.264 (1.10), 1.284 (0.73), 1.294 (2.05), 1.306 (0.63), 1.313 (0.91), 1.328 (13.22), 1.333 (12.88), 1.345 (12.75), 1.361 (0.64), 1.383 (0.54), 1.403 (0.84), 1.416 (0.79), 1.422 (0.82), 1.435 (0.76), 1.445 (0.84), 1.451 (0.67), 1.455 (0.88), 1.465 (0.59), 1.470 (0.60), 1.473 (0.96), 1.492 (1.90), 1.502 (0.69), 1.511 (1.65), 1.520 (0.88), 1.531 (1.76), 1.541 (1.28), 1.545 (1.00), 1.552 (0.99), 1.557 (1.06), 1.563 (0.89), 1.569 (0.87), 1.574 (0.92), 1.582 (0.89), 1.589 (0.81), 1.611 (1.01), 1.623 (0.77), 1.630 (0.86), 1.639 (1.12), 1.649 (1.06), 1.656 (1.24), 1.668 (1.14), 1.676 (0.93), 1.699 (0.67), 1.778 (0.63), 1.794 (0.60), 1.814 (0.70), 1.818 (0.64), 1.822 (0.60), 1.825 (0.60), 1.832 (0.73), 1.848 (0.75), 1.863 (0.60), 1.878 (0.45), 2.111 (0.78), 2.130 (1.59), 2.145 (1.30), 2.150 (1.07), 2.164 (2.35), 2.183 (1.17), 2.275 (1.08), 2.292 (1.26), 2.306 (1.20), 2.322 (0.78), 2.338 (0.62), 2.484 (0.45), 2.728 (0.74), 2.740 (0.88), 2.750 (1.49), 2.763 (1.57), 2.770 (1.19), 2.783 (0.95), 2.825 (1.04), 2.843 (2.33), 2.858 (3.99), 2.865 (1.79), 2.884 (5.40), 3.011 (3.42), 3.037 (2.46), 3.349 (0.80), 3.366 (2.67), 3.382 (2.59), 3.399 (0.75), 3.984 (0.45), 3.995 (0.44), 3.999 (0.50), 4.014 (1.47), 4.026 (3.42), 4.038 (4.98), 4.049 (2.74), 4.068 (0.52), 4.079 (0.74), 4.099 (0.80), 4.107 (4.27), 4.120 (6.44), 4.131 (1.94), 4.134 (1.91), 5.001 (6.15), 5.979 (3.33), 5.995 (3.45), 6.245 (16.00), 6.363 (3.82), 6.365 (3.76), 6.386 (4.01), 6.388 (3.70), 7.248 (3.85), 7.264 (3.99), 7.271 (3.76), 7.287 (3.72), 8.044 (3.90), 8.048 (3.98), 8.545 (3.71), 8.549 (3.68).

### Example 888

### 6-(1-{(3′R)-2-[6-amino-5-(trifluoromethyl)pyridin-3-yl]-6,7-dihydrospiro[pyrazolo[5,1-c][1,4]oxazine-4,3′-pyrrolidin]-1′-yl}ethyl)pyridin-2-ol (Diastereomer 2)

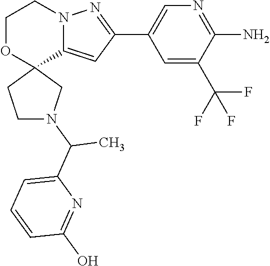 [SMILES: CC(c1cccc(O)n1)N1CC[C@]2(C1)OCCn1nc(-c3cnc(N)c(C(F)(F)F)c3)cc12]

For the preparation of the diastereomeric mixture of the title compound see Example 886. The diastereomers were separated by preparative chiral HPLC (method see Example 887) to give 3.30 mg (90% purity, 20% yield) of the title compound (diastereomer 2 Rₜ=13.73-17.01 min).

Analytical chiral HPLC (method see Example 887): Rₜ=2.52 min

[α]²⁰_{D}: +74.6° (c=1.00, methanol)

LC-MS (Method 1): Rₜ=0.93 min; MS (ESIpos): m/z=462 [M+H]⁺

¹H-NMR (400 MHz, CHLOROFORM-d) δ [ppm]: 0.764 (0.80), 0.783 (1.57), 0.794 (2.99), 0.800 (1.70), 0.811 (4.09), 0.813 (4.79), 0.826 (2.48), 0.828 (2.91), 0.832 (2.53), 0.838 (1.21), 0.845 (2.16), 0.857 (1.76), 0.861 (0.92), 0.864 (0.93), 0.875 (0.99), 0.883 (0.57), 0.971 (0.43), 0.997 (0.41), 1.050 (0.52), 1.066 (1.17), 1.075 (2.98), 1.095 (1.26), 1.099 (2.99), 1.114 (0.79), 1.126 (1.04), 1.129 (0.99), 1.135 (4.21), 1.142 (2.90), 1.150 (8.00), 1.157 (1.89), 1.166 (2.44), 1.185 (15.60), 1.255 (1.52), 1.294 (3.44), 1.323 (14.32), 1.331 (10.59), 1.340 (14.80), 1.363 (2.42), 1.385 (0.60), 1.404 (0.90), 1.409 (0.64), 1.422 (0.90), 1.435 (0.80), 1.443 (0.91), 1.454 (0.88), 1.464 (0.70), 1.472 (0.97), 1.491 (1.76), 1.510 (1.60), 1.530 (2.18), 1.541 (1.58), 1.558 (1.33), 1.574 (1.22), 1.581 (1.17), 1.610 (1.35), 1.638 (1.27), 1.649 (1.25), 1.656 (1.39), 1.668 (1.31), 1.675 (1.14), 1.767 (0.81), 1.782 (0.71), 1.809 (0.74), 1.827 (0.73), 1.843 (0.72), 1.858 (0.60), 1.936 (0.57), 2.085 (0.41), 2.131 (0.81), 2.150 (1.66), 2.166 (1.51), 2.169 (1.19), 2.185 (2.67), 2.204 (1.59), 2.243 (0.41), 2.266 (1.23), 2.278 (1.48), 2.283 (1.59), 2.296 (1.48), 2.312 (0.93), 2.317 (0.89), 2.329 (0.74), 2.680 (0.85), 2.692 (1.03), 2.702 (1.57), 2.714 (1.65), 2.722 (1.23), 2.735 (0.98), 2.826 (1.18), 2.844 (2.63), 2.863 (1.51), 2.867 (2.08), 2.885 (0.88), 2.894 (1.96), 2.921 (7.32), 2.935 (5.90), 2.961 (1.39), 3.320 (0.90), 3.337 (2.93), 3.354 (2.86), 3.370 (0.85), 3.574 (0.47), 3.967 (0.52), 3.978 (0.85), 3.981 (0.78), 3.992 (1.07), 3.997 (1.58), 4.011 (2.86), 4.021 (2.56), 4.031 (4.11), 4.043 (2.43), 4.061 (0.87), 4.073 (1.09), 4.080 (0.43), 4.094 (0.99), 4.100 (3.70), 4.112 (5.43), 4.124 (1.99), 4.127 (1.98), 5.006 (6.65), 5.980 (3.64), 5.996 (3.76), 6.255 (16.00), 6.375 (3.96), 6.377 (4.07), 6.398 (4.41), 6.401 (4.04), 7.252 (4.41), 7.268 (4.25), 7.275 (4.24), 7.292 (3.85), 8.049 (4.26), 8.054 (4.34), 8.555 (4.05), 8.558 (4.05).

### Example 889

### 6-({(3R)-2′-[6-amino-5-(trifluoromethyl)pyridin-3-yl]-5′,6′-dihydrospiro[pyrrolidine-3,4′-pyrrolo[1,2-b]pyrazol]-1-yl}(cyclopropyl)methyl)pyridin-2-ol (Diastereomer 1 and Diastereomer 2)

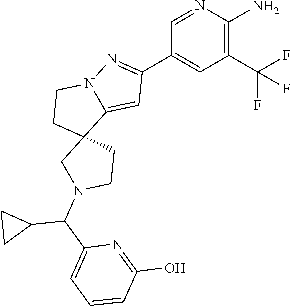 [SMILES: Nc1ncc(-c2cc3n(n2)CC[C@@]32CCN(C(c3cccc(O)n3)C3CC3)C2)cc1C(F)(F)F]

5-{(3R)-1-[Cyclopropyl(6-methoxypyridin-2-yl)methyl]-5′,6′-dihydrospiro[pyrrolidine-3,4′-pyrrolo[1,2-b]pyrazol]-2′-yl}-3-(trifluoromethyl)pyridin-2-amine (17.6 mg, 36.3 μmol, Example 856) was dissolved in acetonitrile (1.2 mL). Sodium iodide (27.2 mg, 182 μmol) was added and chlorotrimethylsilane (23 μL, 180 μmol) was added dropwise. The mixture was stirred for 2 h at 60° C. The reaction mixture was concentrated and diluted with ethyl acetate. The organic phase was washed with brine, dried over sodium sulfate, filtered and concentrated. The residue was purified by preparative TLC (dichloromethane/methanol 9:1 gradient) to give 4.80 mg (95% purity, 27% yield) of the title compound.

LC-MS (Method 1): Rₜ=1.05 min; MS (ESIpos): m/z=472 [M+H]⁺

¹H-NMR (400 MHz, CHLOROFORM-d) δ [ppm]: 0.181 (1.96), 0.192 (2.52), 0.204 (2.62), 0.218 (1.90), 0.441 (0.59), 0.453 (1.39), 0.467 (2.08), 0.479 (2.05), 0.489 (2.64), 0.501 (2.97), 0.512 (4.29), 0.525 (3.35), 0.534 (2.58), 0.546 (1.92), 0.557 (1.07), 0.570 (0.48), 0.750 (0.71), 0.761 (0.89), 0.769 (1.35), 0.785 (1.82), 0.792 (1.82), 0.796 (1.73), 0.807 (1.87), 0.817 (1.51), 0.823 (1.26), 0.838 (1.08), 0.858 (0.55), 0.885 (0.67), 0.974 (0.41), 0.986 (0.79), 0.994 (1.04), 1.006 (1.86), 1.018 (1.78), 1.029 (2.19), 1.038 (1.65), 1.049 (1.50), 1.061 (0.82), 1.259 (3.41), 1.393 (12.23), 2.026 (0.79), 2.046 (1.36), 2.059 (2.01), 2.078 (2.86), 2.096 (2.15), 2.109 (1.58), 2.129 (1.03), 2.202 (1.41), 2.207 (1.38), 2.214 (1.82), 2.224 (2.29), 2.236 (2.71), 2.247 (1.53), 2.257 (1.60), 2.278 (4.87), 2.288 (3.30), 2.301 (4.59), 2.311 (3.04), 2.584 (1.04), 2.601 (1.90), 2.633 (4.65), 2.654 (3.54), 2.660 (2.56), 2.675 (2.46), 2.681 (2.01), 2.694 (2.11), 2.708 (4.66), 2.721 (1.93), 2.733 (6.79), 2.753 (3.08), 2.760 (3.93), 2.772 (1.29), 2.785 (1.45), 2.803 (1.39), 2.824 (1.39), 2.844 (0.82), 2.857 (3.20), 2.880 (2.61), 2.897 (1.79), 2.909 (1.62), 2.919 (1.06), 2.930 (0.83), 3.019 (0.74), 3.031 (0.92), 3.040 (1.23), 3.055 (1.37), 3.074 (0.60), 3.156 (3.40), 3.179 (3.07), 4.126 (0.59), 4.145 (0.83), 4.153 (1.73), 4.161 (0.88), 4.169 (2.77), 4.172 (2.44), 4.181 (1.97), 4.189 (2.88), 4.197 (3.23), 4.201 (2.46), 4.214 (4.61), 4.223 (2.92), 4.236 (3.10), 4.243 (2.67), 4.250 (1.18), 4.257 (2.15), 4.263 (0.95), 4.271 (0.86), 4.284 (0.72), 5.032 (12.87), 5.308 (6.27), 5.993 (3.08), 6.002 (4.11), 6.009 (3.42), 6.017 (3.99), 6.244 (16.00), 6.463 (6.12), 6.486 (6.40), 7.321 (2.50), 7.333 (4.06), 7.337 (2.90), 7.343 (2.73), 7.350 (4.02), 7.356 (4.04), 7.361 (2.65), 7.373 (3.49), 8.124 (8.23), 8.129 (5.54), 8.627 (8.26), 8.631 (8.23), 9.658 (1.42).

### Example 890

### 6-({(3′R)-2-[6-amino-5-(trifluoromethyl)pyridin-3-yl]-6,7-dihydrospiro[pyrazolo[5,1-c][1,4]oxazine-4,3′-pyrrolidin]-1′-yl}(cyclopropyl)methyl)pyridin-2-ol (Diastereomer 1 and Diastereomer 2)

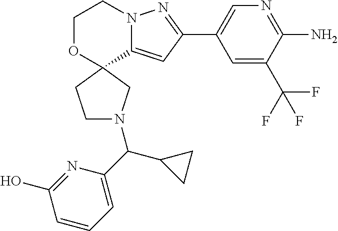 [SMILES: Nc1ncc(-c2cc3n(n2)CCO[C@@]32CCN(C(c3cccc(O)n3)C3CC3)C2)cc1C(F)(F)F]

The compound was prepared similarly to Example 878 using 5-{(3′R)-1′-[cyclopropyl(6-methoxypyridin-2-yl)methyl]-6,7-dihydrospiro[pyrazolo[5,1-c][1,4]oxazine-4,3′-pyrrolidin]-2-yl}-3-(trifluoromethyl)pyridin-2-amine (26.4 mg, 52.7 μmol, Example 857) to give 5.20 mg (95% purity, 19% yield) of the title compound.

LC-MS (Method 1): Rₜ=1.00 min; MS (ESIpos): m/z=488 [M+H]⁺

¹H-NMR (400 MHz, CHLOROFORM-d) 6 [ppm]: 0.204 (3.40), 0.215 (2.80), 0.226 (1.90), 0.493 (2.69), 0.504 (5.68), 0.517 (6.66), 0.528 (6.42), 0.563 (0.91), 0.786 (1.72), 0.804 (3.48), 0.826 (2.62), 0.848 (0.86), 0.885 (0.68), 1.057 (2.65), 1.068 (2.29), 1.079 (2.10), 1.225 (0.50), 1.258 (3.45), 1.783 (6.13), 2.190 (1.00), 2.208 (2.18), 2.225 (2.99), 2.242 (3.56), 2.260 (2.92), 2.279 (0.89), 2.344 (9.28), 2.366 (8.80), 2.377 (3.47), 2.395 (2.10), 2.411 (1.13), 2.824 (1.14), 2.851 (1.19), 2.873 (2.15), 2.893 (7.04), 2.907 (1.58), 2.919 (6.09), 2.945 (1.60), 2.963 (3.40), 2.985 (3.17), 3.003 (1.76), 3.064 (1.25), 3.090 (2.80), 3.118 (1.95), 3.145 (0.84), 3.281 (3.84), 3.308 (3.35), 4.060 (0.75), 4.077 (1.64), 4.095 (4.42), 4.108 (8.66), 4.115 (7.54), 4.126 (5.04), 4.148 (1.33), 4.177 (5.72), 4.188 (9.81), 4.196 (10.09), 5.080 (13.23), 5.308 (16.00), 6.010 (5.32), 6.026 (5.91), 6.317 (10.96), 6.350 (3.44), 6.452 (5.16), 6.475 (5.80), 6.489 (2.52), 7.319 (4.54), 7.336 (4.86), 7.342 (5.67), 7.359 (4.30), 8.122 (9.81), 8.632 (6.83), 8.636 (6.97), 8.648 (3.74), 9.892 (1.04).

### Example 891

### 5-{(3R)-1-[cyclopropyl(1H-1,2,4-triazol-5-yl)methyl]-5′,6′-dihydrospiro[pyrrolidine-3,4′-pyrrolo[1,2-b]pyrazol]-2′-yl}-3-(trifluoromethyl)pyridin-2-amine (Diastereomer 1)

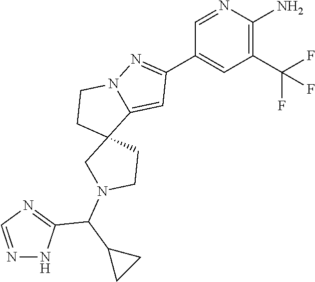 [SMILES: Nc1ncc(-c2cc3n(n2)CC[C@@]32CCN(C(c3ncn[nH]3)C3CC3)C2)cc1C(F)(F)F]

The compound 5-{(3R)-1-[cyclopropyl(1H-1,2,4-triazol-5-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2) was separated into diastereomers by preparative chiral HPLC to give 24.0 mg (99% purity, 13% yield) of the title compound (diastereomer 1 Rₜ=4.1-4.8 min).

Preparative Chiral HPLC Method:

Instrument: PrepCon Labomatic HPLC-3; Column: YMC Amylose SA 5μ, 250×50; eluent A: hexane+0.1 vol % diethylamine; eluent B: ethanol; isocratic: 20% B; flow: 50 mL/min; temperature: 25° C.; UV: 280 nm

Analytical Chiral HPLC Method:

Instrument: Thermo Fisher UltiMate 3000; Column: YMC Amylose SA 3μ, 100×4.6; eluent A: hexane+0.1 vol % diethylamine; eluent B: ethanol; isocratic: 20% B; flow: 1.4 mL/min; temperature: 25° C.; UV: 280 nm

Analytical chiral HPLC Rₜ=3.56 min

[α]²²_{D}: +61.4° (c=1.00, methanol)

LC-MS (Method 1): Rₜ=0.83 min; MS (ESIpos): m/z=445 [M+H]⁺

¹H NMR (DMSO-d₆) δ: 13.79 (br s, 1H), 8.58 (d, 1H), 8.00 (d, 1H), 6.52 (s, 2H), 6.43 (s, 1H), 4.11 (t, 2H), 2.99 (br d, 1H), 2.86 (d, 1H), 2.75-2.82 (m, 1H), 2.55-2.69 (m, 3H), 1.90-2.06 (m, 2H), 1.21-1.32 (m, 1H), 0.59-0.69 (m, 1H), 0.30-0.43 (m, 2H), 0.04-0.13 (m, 1H).

### Example 892

### 5-{(3R)-1-[cyclopropyl(1H-1,2,4-triazol-5-yl)methyl]-5′,6′-dihydrospiro[pyrrolidine-3,4′-pyrrolo[1,2-b]pyrazol]-2′-yl}-3-(trifluoromethyl)pyridin-2-amine (Diastereomer 2)

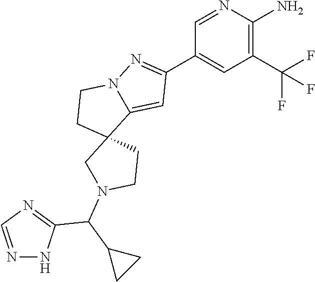 [SMILES: Nc1ncc(-c2cc3n(n2)CC[C@@]32CCN(C(c3ncn[nH]3)C3CC3)C2)cc1C(F)(F)F]

The diastereomers were separated by preparative chiral HPLC (method see Example 891) to give 29.0 mg (100% purity, 16% yield) of the title compound (diastereomer 2 Rₜ=5.2-6.0 min).

Analytical chiral HPLC (method see Example 891): Rₜ=5.10 min

[α]²²_{D}: +41.0° (c=1.00, methanol)

LC-MS (Method 1): Rₜ=0.84 min; MS (ESIpos): m/z=445 [M+H]⁺

¹H NMR (DMSO-d₆) δ: 13.81 (br s, 1H), 8.58 (d, 1H), 8.00 (d, 1H), 6.52 (s, 2H), 6.43 (s, 1H), 4.01-4.14 (m, 2H), 2.91-3.00 (m, 1H), 2.88 (d, 1H), 2.78 (d, 1H), 2.53-2.68 (m, 3H), 1.93-2.04 (m, 2H), 1.21-1.32 (m, 1H), 0.60-0.69 (m, 1H), 0.30-0.45 (m, 2H), 0.05-0.13 (m, 1H).

### Example 893

### 5-{(3R)-1-[(3,3-difluorocyclobutyl)(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (Diastereomer 1 and Diastereomer 2)

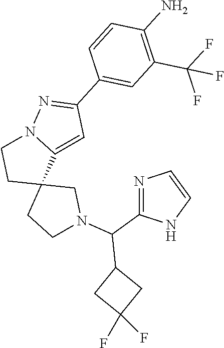 [SMILES: Nc1ccc(-c2cc3n(n2)CC[C@@]32CCN(C(c3ncc[nH]3)C3CC(F)(F)C3)C2)cc1C(F)(F)F]

5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (129 mg, 358 μmol) and (3,3-difluorocyclobutyl)(1H-imidazol-2-yl)methanone (100 mg, 537 μmol, Intermediate 570) were provided in methanol (3 mL). N,N-Diisopropylethylamine (250 μL, 1.4 mmol) and titanium(IV) isopropoxide (320 μL, 1.1 mmol) were added and the mixture was stirred for 2 h at 60° C. Sodium cyanoborohydride (67.5 mg, 1.07 mmol) was added and the mixture was stirred for 6 days at 60° C. The reaction mixture was allowed to cool down, diluted with ethyl acetate and extracted with a saturated aqueous sodium bicarbonate solution and brine. The organic phase was dried over a hydrophobic filter and concentrated. The residue was purified by column chromatography (silica gel, dichloromethane/ethanol gradient). The combined fractions were purified by preparative HPLC to give 60.8 mg (32% yield) of the title compound.

LC-MS (Method 1): Rₜ=1.02 min; MS (ESIpos): m/z=494 [M+H]⁺

¹H-NMR (400 MHz, DMSO-d6) δ [ppm]: 1.850 (0.63), 1.862 (1.43), 1.878 (3.64), 1.896 (5.86), 1.912 (3.49), 1.926 (1.58), 1.937 (0.63), 1.957 (0.48), 2.043 (0.48), 2.094 (1.27), 2.126 (1.11), 2.318 (0.63), 2.323 (1.43), 2.327 (2.06), 2.332 (1.43), 2.336 (0.63), 2.357 (0.95), 2.375 (2.38), 2.389 (2.85), 2.406 (4.12), 2.418 (3.80), 2.435 (3.01), 2.449 (3.96), 2.466 (4.75), 2.518 (6.81), 2.523 (4.91), 2.589 (3.33), 2.611 (4.12), 2.629 (2.38), 2.636 (3.33), 2.652 (6.81), 2.664 (6.81), 2.669 (3.64), 2.674 (3.01), 2.686 (1.74), 2.744 (3.33), 2.769 (6.50), 2.779 (3.49), 2.792 (4.28), 2.812 (1.27), 2.829 (1.90), 2.846 (1.11), 2.852 (1.58), 2.868 (0.63), 3.301 (0.95), 3.378 (1.58), 3.385 (1.11), 3.684 (2.22), 3.708 (4.44), 3.732 (2.06), 4.050 (2.22), 4.061 (4.12), 4.069 (4.75), 4.078 (7.29), 4.096 (3.33), 6.216 (13.47), 6.276 (16.00), 6.514 (15.37), 6.861 (8.87), 7.083 (4.91), 7.087 (5.54), 7.977 (4.75), 7.982 (8.87), 7.987 (5.23), 8.557 (4.44), 8.563 (7.92), 11.915 (4.12).

### Example 894

### 5-{(3R)-1-[(3,3-difluorocyclobutyl)(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (Diastereomer 1)

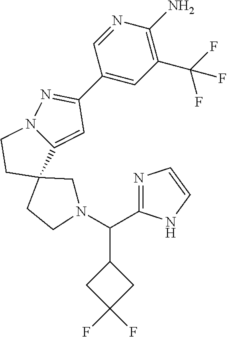 [SMILES: Nc1ncc(-c2cc3n(n2)CC[C@@]32CCN(C(c3ncc[nH]3)C3CC(F)(F)C3)C2)cc1C(F)(F)F]

The diastereomeric mixture of the title compound from Example 893 was separated by preparative chiral HPLC to give 38.5 mg of the title compound (diastereomer 1, Rₜ=4.0-5.2 min).

Preparative Chiral HPLC Method:

Instrument: Sepiatec: Prep SFC100; Column: Chiralpak IG 5μ, 250×50; eluent A: CO2; eluent B: methanol+0.2 vol % diethylamine; isocratic: 20% B; flow: 100 mL/min; temperature: 40° C.; BPR: 150 bar; UV: 271 nm

Analytical Chiral HPLC Method:

Instrument: Waters Acquity UPC2 QDA; Column: Chiralpak IG 3μ, 100×4.6; eluent A: CO2; eluent B: methanol+0.2 vol % diethylamine; isocratic: 20% B; flow: 4 mL/min; temperature: 40° C.; BPR: 100 bar; UV: 280 nm

Analytical chiral HPLC: Rₜ=1.38 min

[α]²²_{D}: +58.2° (c=1.00, methanol)

LC-MS (Method 7): Rₜ=0.80 min; MS (ESIpos): m/z=494 [M+H]⁺

¹H-NMR (400 MHz, DMSO-d6) δ [ppm]: 1.137 (1.27), 1.233 (0.96), 1.352 (0.51), 1.850 (0.57), 1.862 (1.34), 1.877 (1.59), 1.891 (1.47), 1.907 (1.66), 1.910 (1.66), 1.926 (1.34), 1.938 (0.64), 1.958 (0.45), 2.085 (1.02), 2.116 (1.21), 2.318 (1.15), 2.323 (2.61), 2.327 (3.82), 2.332 (2.68), 2.337 (1.15), 2.357 (0.70), 2.375 (1.66), 2.389 (1.98), 2.407 (2.93), 2.424 (1.85), 2.439 (1.08), 2.452 (2.10), 2.468 (4.14), 2.518 (12.69), 2.523 (8.92), 2.589 (3.06), 2.612 (3.63), 2.640 (1.47), 2.660 (2.42), 2.665 (3.31), 2.669 (4.27), 2.673 (3.31), 2.745 (2.29), 2.771 (4.72), 2.780 (2.42), 2.794 (3.19), 3.684 (2.04), 3.708 (2.10), 4.062 (3.25), 4.079 (6.57), 4.097 (3.25), 6.278 (16.00), 6.515 (8.54), 6.863 (3.89), 7.083 (3.44), 7.982 (4.91), 7.987 (4.97), 8.564 (4.53), 8.568 (4.46), 11.907 (2.23).

### Example 895

### 5-{(3R)-1-[(3,3-difluorocyclobutyl)(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (Diastereomer 2)

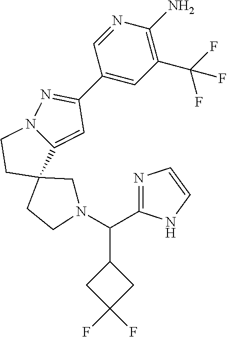 [SMILES: Nc1ncc(-c2cc3n(n2)CC[C@@]32CCN(C(c3ncc[nH]3)C3CC(F)(F)C3)C2)cc1C(F)(F)F]

For the preparation of the diastereomeric mixture of the title compound see Example 893. The diastereomers were separated by preparative chiral HPLC (method see Example 894) to give 22.4 mg of the title compound (diastereomer 2: Rₜ=8.0-9.8 min).

Analytical chiral HPLC (method see Example 894): Rₜ=3.69 min

[α]²²_{D}: +40.6° (c=1.00, methanol)

LC-MS (Method 7): Rₜ=0.80 min; MS (ESIpos): m/z=494 [M+H]⁺

¹H-NMR (400 MHz, DMSO-d6) δ [ppm]: 1.137 (1.41), 1.232 (1.46), 1.880 (2.42), 1.898 (4.88), 1.915 (2.64), 2.084 (0.87), 2.095 (0.68), 2.116 (1.09), 2.318 (0.77), 2.323 (1.82), 2.327 (2.74), 2.332 (1.91), 2.336 (0.87), 2.369 (0.68), 2.387 (1.32), 2.401 (2.01), 2.419 (2.78), 2.432 (2.37), 2.447 (2.92), 2.465 (2.78), 2.518 (8.57), 2.523 (6.34), 2.618 (0.73), 2.630 (1.78), 2.637 (2.05), 2.653 (5.15), 2.660 (4.15), 2.665 (6.29), 2.673 (3.05), 2.678 (1.87), 2.689 (1.55), 2.770 (1.46), 2.815 (1.19), 2.831 (1.91), 2.853 (1.50), 2.871 (0.59), 3.712 (2.19), 3.736 (1.96), 4.052 (2.23), 4.055 (2.10), 4.070 (4.47), 4.090 (2.23), 6.218 (16.00), 6.517 (8.57), 7.059 (0.41), 7.976 (4.88), 7.981 (4.97), 8.558 (4.51), 8.562 (4.42), 11.945 (0.77).

### Example 896

### 5-{1′-[(3,3-difluorocyclobutyl)(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine (Isomer 1)

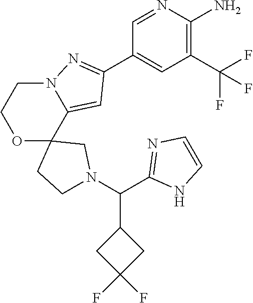 [SMILES: Nc1ncc(-c2cc3n(n2)CCOC32CCN(C(c3ncc[nH]3)C3CC(F)(F)C3)C2)cc1C(F)(F)F]

5-[6,7-Dihydrospiro[pyrazolo[5,1-c][1,4]oxazine-4,3′-pyrrolidin]-2-yl]-3-(trifluoromethyl)pyridin-2-amine hydrogen chloride (1/1) (135 mg, 358 μmol) and (3,3-difluorocyclobutyl)(1H-imidazol-2-yl)methanone (100 mg, 537 μmol, Intermediate 570) were provided in methanol (3 mL). N,N-Diisopropylethylamine (250 μL, 1.4 mmol) and titanium(IV) isopropoxide (320 μL, 1.1 mmol) were added and the mixture was stirred for 2 h at 60° C. Sodium cyanoborohydride (67.5 mg, 1.07 mmol) was added and the mixture was stirred over the weekend at 60° C. The reaction mixture was allowed to cool down, diluted with ethyl acetate and washed with saturated sodium bicarbonate solution and brine. The organic phase was dried over a hydrophobic filter and concentrated. The residue was purified by column chromatography (silica gel, dichloromethane/ethyl acetate gradient) to give 58.0 mg (29% yield) of the title compound as a mixture of isomers.

The isomers were separated by preparative chiral HPLC to give 9.5 mg of the title compound (isomer 1: Rₜ=5.0-6.9 min).

Preparative Chiral HPLC Method:

Instrument: Sepiatec: Prep SFC100; Column: Chiral Art Amylose SA 5μ, 250×30; eluent A: CO2; eluent B: ethanol+0.2 vol % aqueous ammonia (32%); isocratic: 20% B; flow: 100 mL/min; temperature: 40° C.; BPR: 150 bar; UV: 267 nm

Analytical Chiral HPLC Method:

Instrument: Waters Acquity UPC2 QDA; Column: Chiral Art Amylose SA 3μ, 100×4.6; eluent A: CO2; eluent B: ethanol+0.2 vol % aqueous ammonia (32%); isocratic: 20% B; flow: 4 mL/min; temperature: 40° C.; BPR: 1800 psi; UV: 254 nm

Analytical chiral HPLC Rₜ=1.59 min

[α]²⁰_{D}: +55.4° (c=1.00, methanol)

LC-MS (Method 2): Rₜ=0.81 min; MS (ESIpos): m/z=510 [M+H]⁺

¹H-NMR (400 MHz, DMSO-d6) δ [ppm]: 0.798 (2.35), 0.803 (1.04), 0.815 (2.61), 0.822 (2.78), 0.841 (1.74), 0.852 (0.96), 0.860 (0.87), 0.877 (0.78), 0.886 (1.57), 0.896 (0.52), 0.905 (3.30), 0.923 (1.65), 0.984 (0.70), 1.000 (0.61), 1.043 (0.52), 1.053 (0.52), 1.068 (0.78), 1.109 (0.43), 1.137 (2.96), 1.223 (1.30), 1.232 (2.00), 1.333 (0.43), 1.352 (0.78), 1.779 (0.52), 1.872 (0.52), 1.907 (2.17), 2.023 (1.48), 2.036 (1.65), 2.055 (3.13), 2.075 (3.39), 2.085 (3.04), 2.093 (2.61), 2.116 (2.87), 2.174 (0.70), 2.211 (0.43), 2.318 (1.74), 2.323 (3.91), 2.327 (5.48), 2.332 (3.83), 2.337 (1.91), 2.359 (1.22), 2.375 (1.57), 2.389 (1.48), 2.394 (1.65), 2.413 (1.57), 2.518 (16.00), 2.523 (11.65), 2.631 (1.91), 2.651 (2.09), 2.660 (2.61), 2.665 (4.70), 2.669 (6.26), 2.673 (4.43), 2.679 (2.26), 2.739 (2.61), 2.775 (3.22), 2.801 (3.91), 2.964 (2.96), 2.989 (2.35), 3.674 (2.26), 3.697 (2.00), 3.970 (6.43), 3.983 (5.22), 4.041 (5.57), 4.054 (6.52), 4.066 (3.04), 4.559 (0.52), 6.461 (12.26), 6.575 (12.61), 6.855 (2.52), 7.079 (2.61), 8.006 (7.39), 8.011 (7.13), 8.589 (6.78), 8.593 (6.35), 11.950 (2.09).

### Example 897

### 5-{1′-[(3,3-difluorocyclobutyl)(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine (Isomer 2)

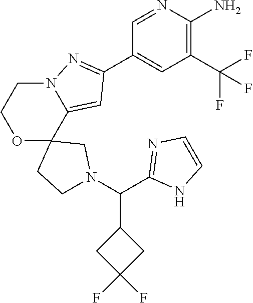 [SMILES: Nc1ncc(-c2cc3n(n2)CCOC32CCN(C(c3ncc[nH]3)C3CC(F)(F)C3)C2)cc1C(F)(F)F]

For the preparation of the isomeric mixture of the title compound see Example 896. The isomers were separated by preparative chiral HPLC (method see Example 896) to give 6.7 mg of the title compound (isomer 2: Rₜ=7.5-9.4 min).

Analytical chiral HPLC (method see Example 896): Rₜ=2.56 min

[α]²⁰_{D}: +40.2° (c=1.00, methanol)

LC-MS (Method 2): Rₜ=0.81 min; MS (ESIpos): m/z=510 [M+H]⁺

¹H-NMR (400 MHz, DMSO-d6) δ [ppm]: 0.798 (2.52), 0.803 (1.22), 0.815 (2.87), 0.822 (3.04), 0.841 (2.00), 0.852 (1.65), 0.860 (1.30), 0.877 (1.22), 0.886 (1.83), 0.896 (0.70), 0.905 (3.48), 0.923 (1.74), 0.950 (0.52), 0.984 (0.96), 1.000 (0.70), 1.029 (0.61), 1.053 (0.78), 1.068 (1.04), 1.113 (0.61), 1.125 (0.78), 1.137 (6.43), 1.223 (1.74), 1.233 (3.04), 1.333 (0.70), 1.352 (0.78), 1.776 (0.43), 1.884 (0.52), 1.907 (3.13), 1.995 (1.48), 2.011 (1.83), 2.028 (2.43), 2.047 (1.48), 2.085 (3.65), 2.088 (2.87), 2.104 (2.87), 2.116 (4.78), 2.141 (1.65), 2.174 (0.96), 2.191 (0.78), 2.211 (0.52), 2.231 (0.43), 2.318 (1.74), 2.323 (3.83), 2.327 (5.39), 2.332 (3.83), 2.337 (1.91), 2.374 (1.22), 2.389 (1.57), 2.394 (1.57), 2.407 (1.65), 2.413 (1.65), 2.518 (16.00), 2.523 (11.83), 2.660 (2.52), 2.665 (5.13), 2.669 (6.96), 2.673 (5.22), 2.679 (2.78), 2.686 (2.00), 2.706 (1.22), 2.754 (3.22), 2.772 (4.61), 2.789 (3.22), 2.835 (2.61), 2.860 (3.65), 2.918 (3.65), 2.944 (2.26), 2.994 (1.30), 3.681 (2.43), 3.704 (2.09), 3.967 (5.13), 3.977 (4.26), 4.039 (5.22), 4.051 (6.87), 4.063 (3.13), 4.559 (1.13), 6.397 (13.04), 6.461 (1.13), 6.578 (12.17), 6.864 (2.96), 7.099 (2.87), 8.001 (6.96), 8.006 (7.30), 8.585 (6.35), 8.589 (6.43), 11.951 (2.26).

### Example 898

### 5-{1′-[(3,3-difluorocyclobutyl)(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine (Isomer 3)

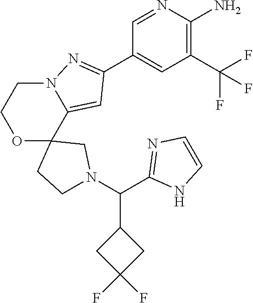 [SMILES: Nc1ncc(-c2cc3n(n2)CCOC32CCN(C(c3ncc[nH]3)C3CC(F)(F)C3)C2)cc1C(F)(F)F]

For the preparation of the isomeric mixture of the title compound see Example 896. The isomers were separated by preparative chiral HPLC (method see Example 896) to give 10.7 mg of the title compound (isomer 3: Rₜ=10.1-13.7 min).

Analytical chiral HPLC (method see Example 896): Rₜ=3.81 min

[α]²⁰_{D}: −71.1° (c=1.00, methanol)

LC-MS (Method 2): Rₜ=0.81 min; MS (ESIpos): m/z=510 [M+H]⁺

¹H-NMR (400 MHz, DMSO-d6) δ [ppm]: 0.798 (4.24), 0.803 (1.86), 0.815 (4.69), 0.822 (4.95), 0.841 (3.01), 0.852 (1.77), 0.860 (1.68), 0.877 (1.50), 0.886 (2.92), 0.896 (0.80), 0.905 (5.83), 0.923 (2.83), 0.950 (0.53), 0.984 (1.06), 1.000 (0.88), 1.018 (0.53), 1.029 (0.80), 1.043 (0.80), 1.053 (0.88), 1.068 (1.15), 1.109 (0.62), 1.125 (1.06), 1.137 (3.27), 1.161 (0.62), 1.195 (0.71), 1.223 (1.86), 1.233 (2.21), 1.256 (1.15), 1.334 (0.71), 1.352 (0.88), 1.393 (0.62), 1.412 (0.53), 1.780 (0.62), 1.871 (0.62), 1.907 (3.01), 2.023 (1.68), 2.036 (1.68), 2.054 (3.09), 2.075 (3.36), 2.084 (3.27), 2.093 (2.74), 2.116 (2.74), 2.130 (1.59), 2.142 (1.50), 2.171 (0.97), 2.191 (0.62), 2.210 (0.71), 2.231 (0.62), 2.318 (1.68), 2.323 (3.80), 2.327 (5.48), 2.332 (3.80), 2.337 (1.94), 2.359 (1.41), 2.374 (1.77), 2.389 (1.77), 2.394 (1.94), 2.407 (1.77), 2.413 (1.86), 2.518 (16.00), 2.523 (12.02), 2.631 (1.94), 2.650 (2.03), 2.660 (2.56), 2.665 (4.60), 2.669 (6.28), 2.673 (4.51), 2.679 (2.30), 2.739 (2.56), 2.775 (3.09), 2.801 (3.89), 2.964 (3.01), 2.988 (2.39), 3.674 (2.12), 3.697 (1.94), 3.970 (6.28), 3.983 (5.13), 4.041 (5.39), 4.054 (6.36), 4.559 (0.53), 6.461 (12.73), 6.574 (12.02), 6.855 (2.83), 7.080 (2.92), 8.005 (7.16), 8.010 (6.98), 8.589 (6.45), 8.593 (6.10), 11.948 (2.03).

### Example 899

### 5-{1′-[(3,3-difluorocyclobutyl)(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine (Isomer 4)

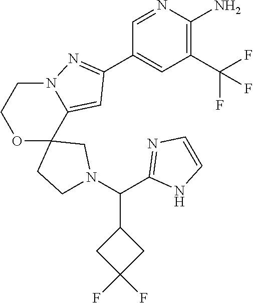 [SMILES: Nc1ncc(-c2cc3n(n2)CCOC32CCN(C(c3ncc[nH]3)C3CC(F)(F)C3)C2)cc1C(F)(F)F]

For the preparation of the isomeric mixture of the title compound see Example 896. The isomers were separated by preparative chiral HPLC (method see Example 896) to give 8.4 mg of the title compound (isomer 4: Rₜ=17.8-23.5 min).

Analytical chiral HPLC (method see Example 896): Rₜ=6.54 min

[α]²⁰_{D}: −43.5° (c=1.00, methanol)

LC-MS (Method 2): Rₜ=0.81 min; MS (ESIpos): m/z=510 [M+H]⁺

¹H-NMR (400 MHz, DMSO-d6) δ [ppm]: 0.724 (0.73), 0.742 (0.45), 0.798 (1.36), 0.804 (0.91), 0.815 (1.64), 0.822 (1.73), 0.834 (1.45), 0.841 (1.91), 0.845 (1.64), 0.853 (3.00), 0.861 (2.82), 0.872 (1.45), 0.877 (2.18), 0.886 (1.18), 0.896 (0.91), 0.905 (1.91), 0.923 (1.18), 0.929 (0.91), 0.950 (0.64), 0.984 (1.09), 1.000 (0.91), 1.030 (1.55), 1.043 (1.09), 1.053 (1.09), 1.068 (1.18), 1.126 (1.55), 1.138 (2.27), 1.195 (1.09), 1.223 (2.64), 1.233 (2.73), 1.281 (1.09), 1.300 (0.91), 1.334 (1.27), 1.352 (1.18), 1.428 (0.55), 1.446 (0.55), 1.776 (0.55), 1.844 (0.45), 1.884 (0.73), 1.907 (3.09), 1.997 (1.36), 2.011 (1.55), 2.028 (2.00), 2.046 (1.09), 2.088 (2.09), 2.104 (2.27), 2.116 (2.18), 2.174 (1.09), 2.332 (4.00), 2.337 (2.00), 2.518 (16.00), 2.523 (11.64), 2.669 (6.64), 2.673 (4.82), 2.679 (2.55), 2.772 (3.09), 2.789 (2.27), 2.836 (1.64), 2.860 (2.36), 2.919 (2.36), 2.943 (1.36), 3.682 (1.45), 3.704 (1.36), 3.967 (3.45), 3.977 (2.82), 4.038 (3.64), 4.050 (4.73), 4.130 (0.45), 6.397 (8.82), 6.578 (8.36), 6.668 (0.45), 6.864 (1.73), 7.098 (1.82), 8.001 (4.82), 8.006 (4.82), 8.585 (4.55), 11.951 (1.27).

### Example 900

### 5-{(3R)-1-[1-(1H-pyrazol-3-yl)propyl]-5′,6′-dihydrospiro[pyrrolidine-3,4′-pyrrolo[1,2-b]pyrazol]-2′-yl}-3-(trifluoromethyl)pyridin-2-amine (Diastereomer 1 and Diastereomer 2)

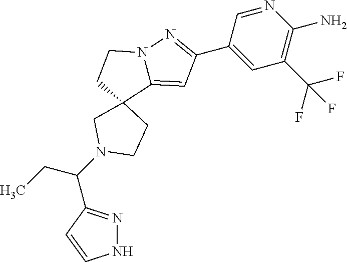 [SMILES: CCC(c1cc[nH]n1)N1CC[C@@]2(CCn3nc(-c4cnc(N)c(C(F)(F)F)c4)cc32)C1]

5-[(3R)-5′,6′-Dihydrospiro[pyrrolidine-3,4′-pyrrolo[1,2-b]pyrazol]-2′-yl]-3-(trifluoromethyl)pyridin-2-amine (166 mg, 514 μmol) and 1-(1H-pyrazol-3-yl)propan-1-one (58.0 mg, 467 μmol, Intermediate 573) were dissolved in methanol (1.6 mL). N,N-Diisopropylethylamine (330 μL, 1.9 mmol) and titanium(IV) isopropoxide (410 μL, 1.4 mmol) were added and the mixture was stirred for 16 h at 60° C. The reaction mixture was allowed to cool down, sodium cyanoborohydride (88.1 mg, 1.40 mmol) was added and stirred for 2 h at 60° C. The reaction mixture was allowed to cool down, diluted with water (1 mL) and stirred for 30 min. The suspension was diluted with MeOH and filtered over celite. The filtrate was concentrated. The residue was dissolved in dichloromethane/methanol (small amount of methanol) and washed twice with saturated sodium bicarbonate solution. The aqueous phase was extracted with dichloromethane/methanol. The combined organic layers were dried over a hydrophobic filter and concentrated. The residue was purified by flash column chromatography (silica gel, dichloromethane/methanol (0-8%) gradient) to give 118.5 mg (100% purity, 53% yield) of the title compound.

LC-MS (Method 1): Rₜ=1.02 min; MS (ESIpos): m/z=432 [M+H]⁺

### Example 901

### 5-{(3R)-1-[(1H-imidazol-2-yl)(1-methylcyclopropyl)methyl]-5′,6′-dihydrospiro[pyrrolidine-3,4′-pyrrolo[1,2-b]pyrazol]-2′-yl}-3-(trifluoromethyl)pyridin-2-amine (Diastereomer 1 and Diastereomer 2)

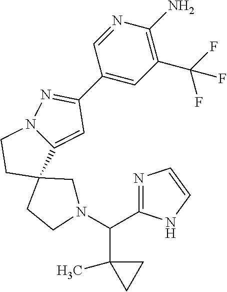 [SMILES: CC1(C(c2ncc[nH]2)N2CC[C@@]3(CCn4nc(-c5cnc(N)c(C(F)(F)F)c5)cc43)C2)CC1]

5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (258 mg, 718 μmol) and (1H-imidazol-2-yl)(1-methylcyclopropyl)methanone (162 mg, 1.08 mmol, Intermediate 569) were provided in methanol (6 mL). N,N-Diisopropylethylamine (500 μL, 2.8 mmol) and titanium(IV) isopropoxide (640 μL, 2.2 mmol) were added and the mixture was stirred for 2 h at 60° C. Sodium cyanoborohydride (135 mg, 2.15 mmol) was added and the mixture was stirred for 7 days at 60° C. 5-[(3R)-5′,6′-dihydrospiro[pyrrolidine-3,4′-pyrrolo[1,2-b]pyrazol]-2′-yl]-3-(trifluoromethyl)pyridin-2-amine hydrogen chloride (1/1) (50 mg, 139 μmol) was added and the reaction mixture was stirred for 3 days at 60° C. The reaction mixture was allowed to cool down, diluted with ethyl acetate and extracted with saturated aqueous sodium bicarbonate solution and brine. The organic phase was dried over a hydrophobic filter and concentrated. The residue was purified by preparative HPLC to give 121 mg (33% yield) of the title compound.

LC-MS (Method 1): Rₜ=1.05 min; MS (ESIpos): m/z=458 [M+H]⁺

¹H-NMR (400 MHz, DMSO-d6) δ [ppm]: −0.035 (0.99), 0.058 (1.44), 0.068 (2.15), 0.077 (2.09), 0.089 (1.51), 0.375 (1.00), 0.388 (1.72), 0.396 (2.22), 0.419 (2.96), 0.441 (2.34), 0.452 (2.17), 0.464 (1.31), 0.485 (0.92), 0.492 (0.97), 0.506 (1.12), 0.543 (0.85), 0.552 (0.87), 0.566 (1.29), 0.578 (0.81), 1.022 (0.40), 1.040 (1.17), 1.055 (16.00), 1.095 (15.14), 1.938 (0.41), 1.957 (0.88), 1.971 (1.73), 1.988 (3.05), 2.003 (3.08), 2.021 (1.69), 2.033 (1.31), 2.046 (1.09), 2.323 (0.87), 2.327 (1.21), 2.331 (0.91), 2.458 (2.71), 2.522 (5.52), 2.589 (6.31), 2.608 (7.77), 2.617 (4.53), 2.636 (3.23), 2.647 (2.39), 2.655 (1.73), 2.664 (2.43), 2.669 (2.39), 2.673 (1.87), 2.679 (1.63), 2.688 (0.77), 2.696 (0.62), 2.824 (2.58), 2.847 (2.28), 2.951 (2.15), 2.974 (2.04), 3.410 (1.05), 4.073 (1.22), 4.088 (2.55), 4.107 (2.83), 4.115 (2.87), 4.122 (2.09), 4.132 (4.51), 4.150 (2.31), 6.449 (0.68), 6.507 (9.97), 6.527 (12.83), 6.590 (9.53), 6.777 (4.61), 6.999 (2.69), 7.040 (2.85), 7.999 (4.76), 8.005 (5.68), 8.013 (3.61), 8.488 (1.24), 8.493 (1.20), 8.594 (6.10), 11.585 (1.81), 11.612 (1.73).

### Example 902

### 5-{(3R)-1-[(1H-imidazol-2-yl)(1-methylcyclopropyl)methyl]-5′,6′-dihydrospiro[pyrrolidine-3,4′-pyrrolo[1,2-b]pyrazol]-2′-yl}-3-(trifluoromethyl)pyridin-2-amine (Diastereomer 1)

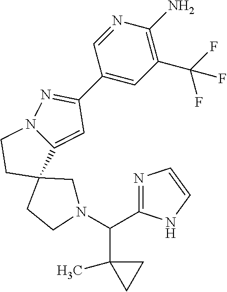 [SMILES: CC1(C(c2ncc[nH]2)N2CC[C@@]3(CCn4nc(-c5cnc(N)c(C(F)(F)F)c5)cc43)C2)CC1]

The diastereomeric mixture of the title compound from Example 901 was separated by preparative chiral HPLC to give 25.0 mg of the title compound (diastereomer 1: Rₜ=3.6-4.6 min).

Preparative Chiral HPLC Method:

Instrument: Sepiatec: Prep SFC100; Column: Chiral Art Amylose SA, 250×30; eluent A: CO2; eluent B: methanol+0.2 vol % aqueous ammonia (32%); isocratic: 20% B; flow: 100 mL/min; temperature: 40° C.; BPR: 150 bar; UV: 271 nm

Analytical Chiral HPLC Method:

Instrument: Waters Acquity UPC2 QDA; Column: Chiral Art Amylose SA 3μ, 100×4.6; eluent A: CO2; eluent B: methanol+0.2 vol % aqueous ammonia (32%); isocratic: 20% B; flow: 4 mL/min; temperature: 40° C.; BPR: 100 bar; UV: 280 nm

Analytical chiral HPLC Rₜ=0.91 min

[α]²²_{D}: +103.6° (c=1.00, methanol)

¹H-NMR (400 MHz, DMSO-d6) δ [ppm]: 0.058 (0.53), 0.065 (0.86), 0.079 (1.36), 0.090 (0.79), 0.100 (0.64), 0.374 (0.49), 0.383 (0.76), 0.387 (0.86), 0.395 (1.32), 0.409 (0.94), 0.415 (1.25), 0.420 (1.14), 0.426 (0.74), 0.440 (1.10), 0.451 (1.06), 0.463 (0.69), 0.484 (0.85), 0.492 (0.88), 0.507 (1.05), 0.519 (0.56), 1.097 (16.00), 1.137 (0.68), 1.956 (0.51), 1.969 (0.82), 1.985 (1.05), 2.004 (0.78), 2.011 (0.74), 2.026 (0.89), 2.033 (0.92), 2.047 (0.92), 2.057 (0.47), 2.323 (0.58), 2.327 (0.82), 2.331 (0.58), 2.458 (2.32), 2.518 (3.96), 2.523 (3.10), 2.560 (0.87), 2.590 (5.61), 2.603 (0.99), 2.609 (1.14), 2.624 (1.06), 2.632 (1.19), 2.648 (1.71), 2.665 (1.83), 2.669 (1.19), 2.673 (0.83), 2.680 (1.21), 2.696 (0.48), 2.953 (2.11), 2.976 (1.89), 4.117 (2.18), 4.134 (4.23), 4.151 (2.09), 6.507 (10.93), 6.527 (5.66), 6.776 (1.07), 6.999 (1.04), 8.007 (3.17), 8.013 (3.24), 8.594 (2.96), 8.598 (2.90), 11.610 (0.86).

### Example 903

### 5-{(3R)-1-[(1H-imidazol-2-yl)(1-methylcyclopropyl)methyl]-5′,6′-dihydrospiro[pyrrolidine-3,4′-pyrrolo[1,2-b]pyrazol]-2′-yl}-3-(trifluoromethyl)pyridin-2-amine (Diastereomer 2)

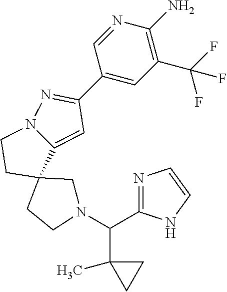 [SMILES: CC1(C(c2ncc[nH]2)N2CC[C@@]3(CCn4nc(-c5cnc(N)c(C(F)(F)F)c5)cc43)C2)CC1]

For the preparation of the diastereomeric mixture of the title compound see Example 901. The diastereomers were separated by preparative chiral HPLC (method see Example 902) to give 22.4 mg of the title compound (diastereomer 2 Rₜ=6.2-8.1 min).

Analytical chiral HPLC (method see Example 902): Rₜ=2.15 min

[α]²²_{D}: −9.6° (c=1.00, methanol)

¹H-NMR (400 MHz, DMSO-d6) δ [ppm]: 0.058 (0.53), 0.065 (0.86), 0.079 (1.36), 0.090 (0.79), 0.100 (0.64), 0.374 (0.49), 0.383 (0.76), 0.387 (0.86), 0.395 (1.32), 0.409 (0.94), 0.415 (1.25), 0.420 (1.14), 0.426 (0.74), 0.440 (1.10), 0.451 (1.06), 0.463 (0.69), 0.484 (0.85), 0.492 (0.88), 0.507 (1.05), 0.519 (0.56), 1.097 (16.00), 1.137 (0.68), 1.956 (0.51), 1.969 (0.82), 1.985 (1.05), 2.004 (0.78), 2.011 (0.74), 2.026 (0.89), 2.033 (0.92), 2.047 (0.92), 2.057 (0.47), 2.323 (0.58), 2.327 (0.82), 2.331 (0.58), 2.458 (2.32), 2.518 (3.96), 2.523 (3.10), 2.560 (0.87), 2.590 (5.61), 2.603 (0.99), 2.609 (1.14), 2.624 (1.06), 2.632 (1.19), 2.648 (1.71), 2.665 (1.83), 2.669 (1.19), 2.673 (0.83), 2.680 (1.21), 2.696 (0.48), 2.953 (2.11), 2.976 (1.89), 4.117 (2.18), 4.134 (4.23), 4.151 (2.09), 6.507 (10.93), 6.527 (5.66), 6.776 (1.07), 6.999 (1.04), 8.007 (3.17), 8.013 (3.24), 8.594 (2.96), 8.598 (2.90), 11.610 (0.86).

### Example 904

### 5-{(3R)-1-[cyclobutyl(4H-1,2,4-triazol-3-yl)methyl]-5′,6′-dihydrospiro[pyrrolidine-3,4′-pyrrolo[1,2-b]pyrazol]-2′-yl}-3-(trifluoromethyl)pyridin-2-amine (Diastereomer 1 and Diastereomer 2)

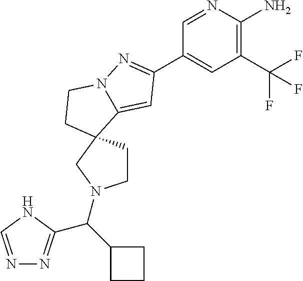 [SMILES: Nc1ncc(-c2cc3n(n2)CC[C@@]32CCN(C(c3nnc[nH]3)C3CCC3)C2)cc1C(F)(F)F]

5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (63.5 mg, 176 μmol) and cyclobutyl(4H-1,2,4-triazol-3-yl)methanone (40.0 mg, 265 μmol, Intermediate 558) were dissolved in methanol (3.8 mL). N,N-Diisopropylethylamine (120 μL, 710 μmol) and titanium(IV) isopropoxide (160 μL, 530 μmol) were added and the mixture was stirred for 3 h at 60° C. The reaction mixture was allowed to cool down, sodium cyanoborohydride (33.3 mg, 529 μmol) was added and the mixture was stirred over night at 60° C. The reaction mixture was allowed to cool down and the precipitate was filtered off. The filtrate was diluted with ethyl acetate and extracted with a saturated sodium bicarbonate solution and water. The organic phase was washed with brine, dried and concentrated. The residue was purified by column chromatography (silica gel NH2, hexane/ethyl acetatel (0-100%) gradient). The combined fractions were purified by preparative HPLC to give 36.0 mg (95% purity, 42% yield) of the title compound.

LC-MS (Method 1): Rₜ=0.92 min; MS (ESIpos): m/z=459 [M+H]⁺

¹H-NMR (400 MHz, DMSO-d6) δ [ppm]: 1.222 (0.98), 1.240 (1.90), 1.258 (0.98), 1.299 (1.05), 1.541 (0.59), 1.564 (0.98), 1.587 (0.79), 1.769 (1.05), 1.798 (0.92), 1.820 (0.52), 1.850 (0.52), 1.870 (0.85), 1.893 (0.92), 1.953 (1.90), 1.974 (2.03), 1.995 (1.05), 2.055 (3.08), 2.189 (0.59), 2.386 (1.31), 2.390 (2.82), 2.395 (4.07), 2.399 (2.82), 2.404 (1.31), 2.586 (16.00), 2.590 (11.08), 2.608 (2.16), 2.659 (1.18), 2.680 (1.25), 2.706 (1.90), 2.727 (1.77), 2.732 (3.28), 2.737 (4.39), 2.741 (3.08), 2.746 (1.51), 2.840 (0.92), 2.948 (0.72), 3.816 (0.85), 3.840 (0.85), 4.085 (0.66), 4.102 (0.66), 4.120 (1.31), 4.136 (2.49), 4.148 (2.62), 4.166 (1.25), 5.827 (5.97), 6.338 (0.79), 6.370 (1.05), 6.583 (5.77), 8.048 (3.41), 8.054 (3.48), 8.585 (0.85), 8.623 (3.15), 13.863 (1.64).

### Example 905

### 5-{(3R)-1-[cyclobutyl(4H-1,2,4-triazol-3-yl)methyl]-5′,6′-dihydrospiro[pyrrolidine-3,4′-pyrrolo[1,2-b]pyrazol]-2′-yl}-3-(trifluoromethyl)pyridin-2-amine (Diastereomer 1)

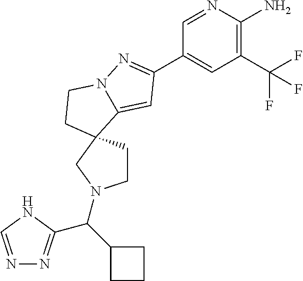 [SMILES: Nc1ncc(-c2cc3n(n2)CC[C@@]32CCN(C(c3nnc[nH]3)C3CCC3)C2)cc1C(F)(F)F]

The title compound from Example 904 was separated into diastereomers by preparative chiral HPLC to give 2.60 mg (90% purity) of the title compound (diastereomer 1 Rₜ=8.5-10.5 min).

Preparative Chiral HPLC Method:

Instrument: Sepiatec: Prep SFC100; Column: Chiralpak IG 5μ 250×50; eluent A: CO2; eluent B: methanol+0.2 vol % aqueous ammonia (32%); isocratic: 25% B; flow: 100 mL/min; temperature: 40° C.; BPR: 150 bar; UV: 280 nm

Analytical Chiral HPLC Method:

Instrument: Agilent: 1260, Aurora SFC-Modul; Column: Chiralpak IG 3μ, 100×4.6; eluent A: CO2; eluent B: methanol+0.2 vol % aqueous ammonia (32%); isocratic: 25% B; flow: 4 mL/min; temperature: 37.5° C.; BPR: 100 bar; UV: 280 nm

Analytical chiral HPLC Rₜ=2.70 min

LC-MS (Method 1): Rₜ=0.92 min; MS (ESIpos): m/z=459 [M+H]⁺

¹H-NMR (400 MHz, METHANOL-d4) 5 [ppm]: −0.028 (1.85), 0.119 (3.76), 0.904 (1.21), 0.920 (1.62), 0.937 (0.89), 1.241 (1.70), 1.259 (3.32), 1.270 (11.60), 1.277 (2.22), 1.285 (1.08), 1.308 (5.96), 1.359 (0.96), 1.389 (0.66), 1.397 (0.73), 1.408 (0.54), 1.419 (0.64), 1.438 (0.73), 1.473 (0.66), 1.577 (0.56), 1.600 (1.58), 1.626 (2.58), 1.649 (2.49), 1.672 (1.04), 1.721 (1.23), 1.729 (1.73), 1.737 (1.70), 1.749 (1.73), 1.757 (1.95), 1.765 (1.54), 1.785 (2.00), 1.812 (1.97), 1.834 (0.96), 1.896 (1.33), 1.918 (2.12), 1.941 (1.85), 1.963 (1.81), 1.977 (1.97), 1.995 (3.89), 2.023 (3.16), 2.032 (6.19), 2.046 (1.97), 2.069 (0.75), 2.084 (1.21), 2.104 (1.79), 2.119 (1.66), 2.131 (1.18), 2.152 (0.83), 2.199 (5.07), 2.217 (1.25), 2.228 (1.64), 2.236 (1.62), 2.245 (1.50), 2.254 (1.37), 2.264 (0.96), 2.283 (0.56), 2.521 (0.91), 2.539 (1.70), 2.554 (2.12), 2.571 (2.39), 2.589 (1.45), 2.647 (3.26), 2.661 (2.60), 2.678 (5.96), 2.694 (2.43), 2.718 (2.72), 2.736 (2.99), 2.878 (2.12), 3.002 (1.21), 3.021 (1.18), 3.797 (4.86), 3.821 (4.57), 4.110 (1.27), 4.128 (1.60), 4.141 (4.47), 4.159 (8.13), 4.176 (4.03), 5.513 (6.84), 6.324 (16.00), 8.111 (6.86), 8.116 (7.02), 8.530 (5.67).

### Example 906

### 5-{(3R)-1-[cyclobutyl(4H-1,2,4-triazol-3-yl)methyl]-5′,6′-dihydrospiro[pyrrolidine-3,4′-pyrrolo[1,2-b]pyrazol]-2′-yl}-3-(trifluoromethyl)pyridin-2-amine (Diastereomer 2)

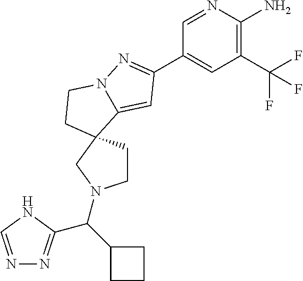 [SMILES: Nc1ncc(-c2cc3n(n2)CC[C@@]32CCN(C(c3nnc[nH]3)C3CCC3)C2)cc1C(F)(F)F]

For the preparation of the diastereomeric mixture of the title compound see Example 904. The diastereomers were separated by preparative chiral HPLC (method see Example 905) to give 3.30 mg (90% purity) of the title compound (diastereomer 2 Rₜ=16.3-19.3 min).

Analytical chiral HPLC (method see Example 905): Rₜ=4.48 min

LC-MS (Method 1): Rₜ=0.92 min; MS (ESIpos): m/z=459 [M+H]⁺

¹H-NMR (400 MHz, METHANOL-d4) 5 [ppm]: 0.101 (3.27), 0.884 (1.24), 0.901 (1.78), 1.120 (0.57), 1.136 (0.57), 1.223 (2.05), 1.241 (4.31), 1.251 (8.03), 1.259 (2.70), 1.267 (1.65), 1.289 (6.55), 1.378 (1.00), 1.400 (0.67), 1.441 (1.11), 1.562 (0.59), 1.585 (1.63), 1.611 (2.65), 1.634 (2.50), 1.658 (1.07), 1.707 (1.87), 1.716 (1.76), 1.727 (1.85), 1.736 (1.74), 1.770 (1.85), 1.797 (2.11), 1.819 (1.04), 1.859 (0.57), 1.882 (1.39), 1.904 (2.29), 1.928 (1.98), 1.937 (1.28), 1.951 (1.28), 1.968 (1.37), 1.988 (2.48), 2.014 (10.36), 2.029 (3.61), 2.037 (3.77), 2.059 (2.70), 2.073 (2.11), 2.106 (0.65), 2.180 (3.13), 2.230 (1.76), 2.238 (1.72), 2.248 (1.65), 2.510 (0.92), 2.529 (1.41), 2.543 (2.39), 2.562 (2.57), 2.577 (1.91), 2.595 (1.54), 2.612 (2.33), 2.629 (3.57), 2.644 (1.44), 2.660 (14.69), 2.722 (2.85), 2.743 (2.07), 2.827 (1.63), 2.904 (1.48), 2.986 (1.35), 3.012 (1.44), 3.789 (3.63), 3.814 (3.40), 4.074 (0.85), 4.091 (2.29), 4.103 (3.00), 4.110 (2.81), 4.117 (5.16), 4.122 (4.40), 4.131 (4.25), 4.136 (5.11), 4.151 (2.85), 4.178 (0.55), 5.495 (4.31), 6.298 (16.00), 8.046 (0.55), 8.094 (7.32), 8.098 (7.62), 8.516 (5.40).

### Example 907

### 5-{(3′R)-1′-[cyclohexyl(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine (Diastereomer 1 and Diastereomer 2)

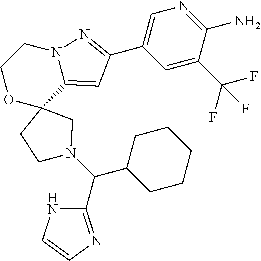 [SMILES: Nc1ncc(-c2cc3n(n2)CCO[C@@]32CCN(C(c3ncc[nH]3)C3CCCCC3)C2)cc1C(F)(F)F]

5-[(3′R)-6,7-Dihydrospiro[pyrazolo[5,1-c][1,4]oxazine-4,3′-pyrrolidin]-2-yl]-3-(trifluoromethyl)pyridin-2-amine hydrogen chloride (1/1) (78.1 mg, 90% purity, 187 μmol) and cyclohexyl(1H-imidazol-2-yl)methanone (50.0 mg, 281 μmol, Intermediate 557) were dissolved 5 in methanol (3.2 mL). N,N-Diisopropylethylamine (130 μL, 751 μmol) and titanium(IV) isopropoxide (170 μL, 560 μmol) were added and the mixture was stirred for 3 h at 60° C. The reaction mixture was allowed to cool down, sodium cyanoborohydride (35 mg, 561 μmol) was added and the mixture was stirred over night at 60° C. The reaction mixture was allowed to cool down and the precipitate was filtered off. The filtrate was diluted with ethyl acetate and extracted with a saturated sodium bicarbonate solution and water. The organic phase was washed with brine, dried and concentrated. The residue was purified by column chromatography (silica gel NH2, hexane/ethyl acetatel (0-100%) and ethyl acetate/ethanol (0-100%) gradient). The combined fractions were purified by preparative HPLC to give 10.0 mg (95% purity, 10% yield) of the title compound.

LC-MS (Method 1): Rₜ=1.11 min; MS (ESIpos): m/z=503 [M+H]⁺

¹H-NMR (400 MHz, DMSO-d6) δ [ppm]: 0.822 (0.99), 0.918 (0.63), 0.935 (0.99), 0.965 (0.99), 0.992 (0.49), 1.162 (1.27), 1.184 (1.48), 1.221 (1.27), 1.239 (1.55), 1.299 (2.54), 1.420 (1.20), 1.633 (2.04), 1.659 (1.83), 1.719 (0.70), 1.751 (0.99), 1.920 (0.78), 1.945 (0.92), 2.014 (0.85), 2.032 (0.85), 2.055 (2.04), 2.062 (1.34), 2.091 (1.27), 2.110 (2.26), 2.130 (1.90), 2.146 (0.78), 2.152 (1.76), 2.394 (4.44), 2.398 (3.10), 2.404 (1.41), 2.585 (16.00), 2.590 (10.85), 2.632 (0.92), 2.654 (1.13), 2.676 (0.70), 2.692 (0.49), 2.736 (4.37), 2.740 (2.96), 2.745 (1.27), 2.823 (0.78), 2.856 (2.26), 2.870 (1.97), 2.881 (2.47), 2.895 (1.90), 3.009 (1.41), 3.034 (1.06), 3.083 (1.90), 3.108 (1.62), 3.518 (2.82), 3.541 (2.82), 4.035 (3.88), 4.047 (3.17), 4.108 (3.31), 4.121 (4.09), 4.133 (1.83), 5.827 (10.22), 6.407 (5.43), 6.461 (7.82), 6.644 (6.70), 6.909 (2.26), 7.096 (1.83), 7.119 (1.34), 8.060 (3.95), 8.064 (3.95), 8.552 (0.49), 8.640 (4.02), 11.803 (1.69).

### Example 908

### 5-{(3R)-1-[cyclohexyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (Diastereomer 1 and Diastereomer 2)

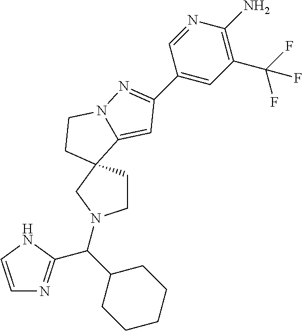 [SMILES: Nc1ncc(-c2cc3n(n2)CC[C@@]32CCN(C(c3ncc[nH]3)C3CCCCC3)C2)cc1C(F)(F)F]

5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (67.3 mg, 187 μmol) and cyclohexyl(1H-imidazol-2-yl)methanone (50.0 mg, 281 μmol, Intermediate 557) were dissolved in methanol (4 mL). N,N-Diisopropylethylamine (130 μL, 750 μmol) and titanium(IV) isopropoxide (170 μL, 560 μmol) were added and the mixture was stirred for 3 h at 60° C. The reaction mixture was allowed to cool down, sodium cyanoborohydride (35 mg, 561 μmol) was added and the mixture was stirred over night at 60° C. The reaction mixture was allowed to cool down and the precipitate was filtered off. The filtrate was diluted with ethyl acetate and extracted with a saturated sodium bicarbonate solution and water. The organic phase was washed with brine, dried and concentrated. The residue was purified by column chromatography (silica gel NH2, hexane/ethyl acetatel (0-100%) and ethyl acetate/ethanol (0-100%) gradient). The combined fractions were purified by preparative HPLC to give 11.0 mg (90% purity, 11% yield) of the title compound.

LC-MS (Method 1): Rₜ=1.15 min; MS (ESIpos): m/z=487 [M+H]⁺

¹H-NMR (400 MHz, DMSO-d6) δ [ppm]: −0.020 (1.80), 0.765 (0.69), 0.793 (0.83), 0.822 (0.42), 0.899 (0.48), 0.942 (0.69), 0.975 (0.76), 1.001 (0.48), 1.110 (0.62), 1.138 (1.32), 1.163 (1.39), 1.202 (0.76), 1.220 (0.83), 1.239 (0.83), 1.280 (2.08), 1.356 (0.97), 1.388 (0.83), 1.620 (1.45), 1.641 (1.45), 1.742 (0.76), 1.897 (1.66), 1.915 (2.63), 1.929 (1.87), 1.945 (1.32), 2.036 (0.83), 2.053 (0.83), 2.084 (0.76), 2.384 (1.52), 2.408 (0.69), 2.423 (0.83), 2.440 (1.32), 2.456 (1.04), 2.470 (1.32), 2.488 (1.73), 2.497 (2.35), 2.566 (16.00), 2.571 (10.87), 2.621 (1.18), 2.643 (1.59), 2.655 (0.83), 2.682 (1.45), 2.726 (1.32), 2.750 (1.52), 2.772 (0.83), 2.867 (1.94), 2.889 (1.39), 3.487 (0.42), 3.507 (1.80), 3.531 (1.73), 4.106 (2.08), 4.124 (4.09), 4.141 (2.08), 5.808 (11.01), 6.228 (5.75), 6.290 (5.68), 6.564 (5.33), 6.890 (1.39), 7.076 (1.04), 7.229 (0.42), 8.021 (3.39), 8.026 (3.39), 8.481 (0.42), 8.597 (3.12), 8.602 (3.12), 11.757 (0.83).

### Example 909

### 5-{(3R)-1-[1-(4-chloro-1H-imidazol-2-yl)propyl]-5′,6′-dihydrospiro[pyrrolidine-3,4′-pyrrolo[1,2-b]pyrazol]-2′-yl}-3-(trifluoromethyl)pyridin-2-amine (Diastereomer 1 and Diastereomer 2)

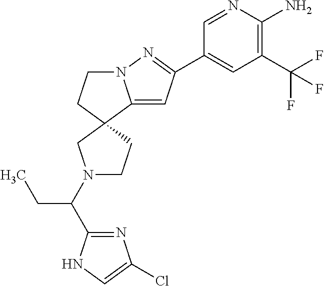 [SMILES: CCC(c1nc(Cl)c[nH]1)N1CC[C@@]2(CCn3nc(-c4cnc(N)c(C(F)(F)F)c4)cc32)C1]

(3R)-2′-[6-Amino-5-(trifluoromethyl)pyridin-3-yl]-5′,6′-dihydrospiro[pyrrolidin-1-ium-3,4′-pyrrolo[1,2-b]pyrazole] chloride (51.6 mg, 144 μmol) and 4-chloro-2-propanoyl-1H-imidazol-1-ium chloride (60.0 mg, 70% purity, 215 μmol, Intermediate 566) were dissolved in methanol (0.9 mL). N,N-Diisopropylethylamine (130 μL, 720 μmol) and titanium(IV) isopropoxide (130 μL, 430 μmol) were added and the mixture was stirred for 16 h at 60° C. The reaction mixture was cooled to room temperature and sodium cyanoborohydride (27.1 mg, 431 μmol) was added and stirred for 2 h at 60° C. The reaction mixture was allowed to cool down, diluted with water and stirred for 30 min. The suspension was diluted with MeOH and filtered over celite. The filtrate was concentrated. The residue was dissolved in ethyl acetate and washed with a saturated sodium bicarbonate solution and brine. The organic phase was dried over sodium sulfate, filtered and concentrated. The residue was purified by column chromatography (silica gel, dichloromethane/methanol (0-15%) gradient) to give 46.3 mg (95% purity, 66% yield) of the title compound.

LC-MS (Method 1): Rₜ=1.04 min; MS (ESIpos): m/z=466 [M+H]⁺

¹H-NMR (400 MHz, CHLOROFORM-d) δ [ppm]: 0.826 (2.33), 0.836 (2.63), 0.844 (5.41), 0.855 (5.46), 0.863 (2.71), 0.874 (2.49), 1.407 (0.27), 1.446 (0.34), 1.465 (0.45), 1.484 (0.21), 1.729 (0.30), 1.747 (0.45), 1.762 (0.70), 1.774 (0.58), 1.781 (0.73), 1.796 (0.59), 1.814 (0.47), 1.833 (0.27), 1.912 (0.67), 1.928 (0.95), 1.942 (0.96), 1.947 (0.97), 1.961 (1.01), 1.976 (0.73), 1.980 (0.69), 1.992 (0.66), 2.004 (0.68), 2.010 (0.64), 2.022 (0.92), 2.037 (0.96), 2.042 (0.89), 2.058 (0.96), 2.074 (0.51), 2.123 (0.47), 2.138 (0.58), 2.144 (0.60), 2.158 (0.67), 2.165 (0.54), 2.170 (0.49), 2.181 (0.72), 2.186 (0.66), 2.201 (0.57), 2.213 (0.41), 2.219 (0.39), 2.234 (0.33), 2.541 (0.35), 2.558 (0.57), 2.574 (1.09), 2.591 (1.45), 2.600 (0.81), 2.635 (1.35), 2.641 (0.91), 2.649 (1.39), 2.655 (1.11), 2.673 (1.50), 2.688 (0.37), 2.696 (0.37), 2.713 (1.29), 2.736 (2.13), 2.755 (0.86), 2.772 (0.60), 2.799 (1.23), 2.822 (0.73), 2.879 (0.34), 2.909 (1.43), 2.922 (0.82), 2.931 (1.30), 2.944 (0.61), 2.966 (0.26), 3.521 (0.79), 3.527 (0.88), 3.532 (0.85), 3.538 (0.88), 3.543 (0.82), 3.549 (0.78), 4.126 (0.21), 4.133 (0.25), 4.141 (0.28), 4.146 (0.32), 4.153 (1.00), 4.160 (1.02), 4.169 (1.77), 4.173 (1.29), 4.177 (1.18), 4.180 (1.39), 4.185 (1.61), 4.188 (1.67), 4.197 (1.80), 4.204 (1.47), 4.210 (0.55), 4.217 (0.92), 4.224 (0.33), 4.231 (0.43), 4.244 (0.22), 5.043 (3.95), 5.084 (0.19), 5.308 (16.00), 6.165 (5.12), 6.187 (0.18), 6.200 (4.98), 6.217 (0.18), 6.886 (2.98), 6.923 (2.97), 8.105 (1.72), 8.110 (2.89), 8.116 (1.64), 8.598 (1.58), 8.604 (2.08), 8.611 (1.51), 9.317 (0.25), 9.414 (0.22).

### Example 910

### 5-{(3R)-1-[(3,3-difluorocyclobutyl)(4H-1,2,4-triazol-3-yl)methyl]-5′,6′-dihydrospiro[pyrrolidine-3,4′-pyrrolo[1,2-b]pyrazol]-2′-yl}-3-(trifluoromethyl)pyridin-2-amine (Diastereomer 1 and Diastereomer 2)

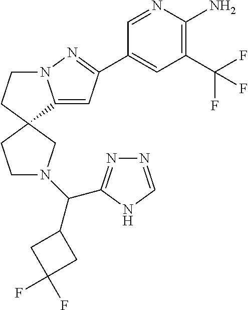 [SMILES: Nc1ncc(-c2cc3n(n2)CC[C@@]32CCN(C(c3nnc[nH]3)C3CC(F)(F)C3)C2)cc1C(F)(F)F]

5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (216 mg, 601 μmol) and (3,3-difluorocyclobutyl)(1H-1,2,4-triazol-5-yl)methanone (135 mg, 721 μmol, Intermediate 565) were provided in methanol (5 mL). N,N-Diisopropylethylamine (420 μL, 2.4 mmol) and titanium(IV) isopropoxide (530 μL, 1.8 mmol) were added and the mixture was stirred for 2 h at 60° C. Sodium cyanoborohydride (113 mg, 1.80 mmol) was added and the mixture was stirred for 3 days at 60° C. The reaction mixture was allowed to cool down, diluted with ethyl acetate and washed with saturated aqueous sodium bicarbonate solution and brine. The organic phase was dried over a hydrophobic filter and concentrated. The residue was purified several times by column chromatography (silica gel, dichloromethane/ethyl acetate/ethanol and ethyl acetate/ethanol gradient) to give 57.0 mg (19% yield) of the title compound.

LC-MS (Method 2): Rₜ=0.76 min; MS (ESIpos): m/z=495 [M+H]⁺

¹H-NMR (400 MHz, DMSO-d6) δ [ppm]: 0.798 (0.92), 0.802 (1.05), 0.814 (0.98), 0.821 (1.05), 0.840 (0.46), 0.886 (0.52), 0.904 (1.18), 0.923 (0.52), 1.035 (6.23), 1.052 (16.00), 1.070 (8.26), 1.089 (0.92), 1.160 (1.18), 1.232 (0.92), 1.353 (0.59), 1.842 (0.46), 1.863 (1.11), 1.881 (2.03), 1.898 (1.84), 1.912 (1.64), 2.121 (0.72), 2.147 (0.72), 2.318 (1.11), 2.323 (2.43), 2.327 (3.48), 2.331 (2.43), 2.337 (1.11), 2.364 (0.46), 2.378 (0.92), 2.395 (1.57), 2.409 (1.90), 2.437 (2.36), 2.454 (2.69), 2.518 (12.59), 2.523 (8.52), 2.615 (1.25), 2.636 (1.97), 2.654 (1.84), 2.659 (1.84), 2.665 (3.08), 2.669 (4.20), 2.673 (3.41), 2.678 (2.62), 2.703 (2.36), 2.725 (1.97), 2.746 (2.82), 2.768 (2.95), 2.786 (3.41), 2.807 (2.03), 2.820 (1.05), 2.843 (0.85), 2.864 (0.98), 2.882 (0.59), 3.364 (0.66), 3.404 (1.05), 3.417 (1.05), 3.422 (3.61), 3.435 (3.74), 3.440 (3.15), 3.452 (3.34), 3.457 (1.11), 3.469 (1.11), 3.817 (0.98), 3.839 (1.11), 3.901 (0.52), 3.920 (0.92), 3.944 (0.59), 4.063 (1.97), 4.079 (3.87), 4.089 (2.49), 4.344 (2.49), 4.357 (4.79), 4.370 (2.36), 5.759 (3.48), 6.245 (4.20), 6.257 (2.75), 6.298 (4.13), 6.308 (2.69), 6.509 (5.64), 6.520 (4.33), 7.938 (2.56), 7.988 (4.66), 8.522 (3.67), 8.527 (3.67), 8.562 (3.54), 13.920 (2.56), 13.947 (1.38).

### Example 911

### 5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}-3-(trifluoromethyl)pyrazin-2-amine (Diastereomer 1 and Diastereomer 2)

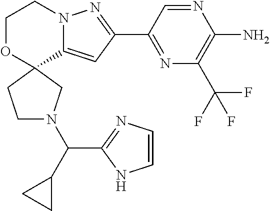 [SMILES: Nc1ncc(-c2cc3n(n2)CCO[C@@]32CCN(C(c3ncc[nH]3)C3CC3)C2)nc1C(F)(F)F]

5-[(3′R)-6,7-Dihydrospiro[pyrazolo[5,1-c][1,4]oxazine-4,3′-pyrrolidin]-2-yl]-3-(trifluoromethyl)pyrazin-2-amine (100 mg, 90% purity, 264 μmol) and cyclopropyl(1H-imidazol-2-yl)methanone (54.0 mg, 397 μmol) were dissolved in methanol (4.6 mL). N,N-Diisopropylethylamine (184 μL, 1.06 mmol) and titanium(IV) isopropoxide (230 μL, 790 μmol) were added and the mixture was stirred for 3 h at 60° C. The reaction mixture was allowed to cool down, sodium cyanoborohydride (49.8 mg, 0.7 mmol) was added and the mixture was stirred for 20 h at 60° C. Sodium cyanoborohydride (24.9 mg, 0.35 mmol) was added and stirred for 20 h at 60° C. A second batch of sodium cyanoborohydride (24.9 mg, 0.35 mmol) was added and stirred for 20 h at 60° C. Sodium cyanoborohydride (24.9 mg, 0.35 mmol) was added and stirred for 3 days at 60° C. The reaction mixture was allowed to cool down, diluted with water and stirred for 15 min. The precipitate was filtered off and washed with methanol. The filtrate was concentrated. The residue was purified by preparative HPLC to give 10.0 mg (90% purity, 7% yield) of the title compound.

LC-MS (Method 1): Rₜ=0.92 min; MS (ESIpos): m/z=462 [M+H]⁺

¹H-NMR (400 MHz, DMSO-d6) δ [ppm]: −0.134 (0.19), 0.251 (2.22), 0.281 (1.72), 0.293 (2.17), 0.304 (2.46), 0.316 (2.32), 0.327 (1.53), 0.412 (0.54), 0.432 (0.54), 0.559 (2.13), 0.781 (0.79), 0.933 (0.83), 0.944 (0.93), 0.949 (1.12), 0.959 (1.22), 0.980 (0.43), 1.069 (0.37), 1.162 (4.84), 1.283 (0.35), 1.684 (2.98), 1.834 (0.31), 1.918 (0.66), 1.939 (1.10), 1.951 (0.81), 1.973 (1.32), 1.993 (0.91), 2.025 (1.30), 2.040 (1.97), 2.056 (2.61), 2.075 (2.26), 2.105 (2.92), 2.118 (2.61), 2.227 (3.08), 2.259 (1.39), 2.263 (1.18), 2.287 (0.60), 2.471 (3.71), 2.601 (1.78), 2.617 (3.19), 2.626 (3.50), 2.639 (4.59), 2.649 (3.48), 2.665 (2.28), 2.699 (1.01), 2.714 (2.22), 2.740 (3.19), 2.777 (1.06), 2.802 (0.64), 2.845 (1.37), 2.864 (2.48), 2.896 (2.69), 2.927 (2.94), 2.939 (2.26), 3.097 (3.08), 3.109 (2.03), 3.133 (2.77), 3.159 (2.48), 3.496 (0.62), 3.611 (0.46), 3.640 (0.31), 3.757 (0.31), 3.893 (0.77), 3.910 (1.22), 3.924 (2.28), 3.944 (2.92), 3.957 (3.42), 3.972 (5.44), 3.982 (5.40), 3.990 (5.28), 4.002 (3.27), 4.042 (7.49), 4.054 (6.75), 4.065 (5.40), 4.073 (3.85), 4.107 (1.22), 5.746 (0.15), 6.038 (0.41), 6.043 (0.37), 6.095 (0.21), 6.448 (2.03), 6.460 (14.14), 6.525 (6.46), 6.601 (0.48), 6.634 (1.06), 6.655 (0.97), 6.684 (1.03), 6.696 (0.95), 6.890 (14.76), 7.009 (0.81), 7.247 (0.68), 7.318 (0.60), 8.119 (0.41), 8.142 (0.35), 8.548 (0.27), 8.674 (16.00), 11.816 (0.58).

### Example 912

### 5-{(3R)-1-[(1H-imidazol-2-yl)(1-methylcyclobutyl)methyl]-5′,6′-dihydrospiro[pyrrolidine-3,4′-pyrrolo[1,2-b]pyrazol]-2′-yl}-3-(trifluoromethyl)pyridin-2-amine (Diastereomer 1 and Diastereomer 2)

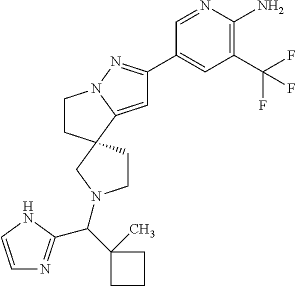 [SMILES: CC1(C(c2ncc[nH]2)N2CC[C@@]3(CCn4nc(-c5cnc(N)c(C(F)(F)F)c5)cc43)C2)CCC1]

5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (49.7 mg, 138 μmol) and (1H-imidazol-2-yl)(1-methylcyclobutyl)methanone (34.0 mg, 207 μmol, Intermediate 556) were dissolved in methanol (3 mL). N,N-Diisopropylethylamine (96 μL, 550 μmol) and titanium(IV) isopropoxide (120 μL, 410 μmol) were added and the mixture was stirred for 3 h at 60° C. The reaction mixture was allowed to cool down, sodium cyanoborohydride (26.0 mg, 414 μmol) was added and the mixture was stirred over night at 60° C. Titanium(IV) isopropoxide (120 μL, 410 μmol) and sodium cyanoborohydride (26.0 mg, 414 μmol) were added again and stirred over the weekend at 60° C. The reaction mixture was allowed to cool down and the precipitate was filtered off. The filtrate was diluted with ethyl acetate and washed with a saturated sodium bicarbonate solution and water. The organic phase was washed with brine, dried and concentrated. The residue was purified by column chromatography (silica gel NH2, hexane/ethyl acetatel (0-100%) and ethyl acetate/ethanol (0-100%) gradient). The combined fractions were purified by preparative HPLC to give 1.00 mg (90% purity, 1% yield) of the title compound.

LC-MS (Method 1): Rₜ=1.11 min; MS (ESIpos): m/z=472 [M+H]⁺

¹H-NMR (400 MHz, METHANOL-d4) 5 [ppm]: 0.119 (4.98), 0.902 (2.90), 0.920 (3.16), 0.937 (1.71), 1.008 (0.57), 1.059 (0.83), 1.151 (1.88), 1.241 (1.45), 1.260 (2.42), 1.277 (2.11), 1.308 (12.30), 1.327 (4.67), 1.335 (3.93), 1.363 (15.86), 1.393 (16.00), 1.408 (2.19), 1.419 (1.94), 1.425 (1.96), 1.474 (2.08), 1.541 (0.94), 1.614 (0.91), 1.650 (0.85), 1.760 (1.65), 1.786 (1.82), 1.809 (1.00), 1.831 (0.94), 1.859 (1.57), 1.927 (0.54), 1.965 (0.97), 1.984 (3.10), 2.010 (1.94), 2.032 (3.96), 2.046 (2.05), 2.062 (2.33), 2.079 (1.77), 2.097 (1.62), 2.112 (1.71), 2.119 (2.22), 2.132 (2.65), 2.147 (2.93), 2.163 (2.28), 2.175 (1.74), 2.199 (2.05), 2.226 (2.33), 2.254 (1.59), 2.274 (0.97), 2.292 (0.60), 2.479 (2.14), 2.501 (2.39), 2.561 (2.14), 2.583 (3.56), 2.601 (2.05), 2.608 (1.54), 2.615 (2.70), 2.622 (2.73), 2.630 (3.44), 2.634 (3.30), 2.645 (3.33), 2.659 (1.91), 2.666 (2.25), 2.678 (2.82), 2.704 (0.57), 2.716 (1.08), 2.722 (1.14), 2.731 (1.51), 2.736 (1.82), 2.747 (3.27), 2.757 (1.48), 2.769 (3.42), 2.773 (3.36), 2.796 (2.19), 3.545 (4.93), 3.553 (4.87), 4.111 (0.85), 4.118 (0.54), 4.124 (1.17), 4.128 (1.00), 4.139 (1.31), 4.144 (1.68), 4.150 (1.45), 4.156 (1.85), 4.165 (2.31), 4.171 (2.96), 4.174 (2.45), 4.178 (2.45), 4.182 (2.08), 4.190 (1.79), 4.198 (1.77), 5.513 (7.60), 6.465 (8.88), 6.583 (8.85), 7.004 (12.67), 7.021 (11.64), 8.140 (3.47), 8.144 (5.49), 8.563 (3.02), 8.567 (2.96), 8.576 (2.68), 8.580 (2.56).

### Example 913

### (3R)-2′-{5-amino-6-[(1R)-1-phenylethoxy]pyrazin-2-yl}-N-ethyl-5′,6′-dihydrospiro[pyrrolidine-3,4′-pyrrolo[1,2-b]pyrazole]-1-carboxamide

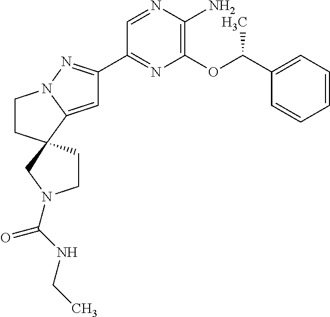 [SMILES: CCNC(=O)N1CC[C@@]2(CCn3nc(-c4cnc(N)c(O[C@H](C)c5ccccc5)n4)cc32)C1]

(3R)-1-(Ethylcarbamoyl)-5′,6′-dihydrospiro[pyrrolidine-3,4′-pyrrolo[1,2-b]pyrazol]-2′-yl trifluoromethanesulfonate (43.0 mg, 112 μmol), 3-[(1R)-1-phenylethoxy]-5-(4,4,5,5-tetramethyl-1,3,2-dioxaborolan-2-yl)pyrazin-2-amine (42.2 mg, 124 μmol) and potassium phosphate (670 μL, 0.50 M, 340 μmol) were dissolved in degassed 1,4-dioxane (1.7 mL) under argon. XPhos Pd G2 (4.42 mg, 5.62 μmol) was added and the mixture was stirred for 2 h at 100° C. The reaction mixture was allowed to cool down, diluted with water and extracted with ethyl acetate. The combined organic layers were washed with brine, dried over sodium sulfate, filtered and concentrated. The residue was purified by preparative HPLC to give 14.0 mg (95% purity, 26% yield) of the title compound.

LC-MS (Method 1): Rₜ=0.98 min; MS (ESIpos): m/z=448 [M+H]⁺

¹H-NMR (400 MHz, DMSO-d6) δ [ppm]: 1.026 (7.20), 1.044 (16.00), 1.062 (7.35), 1.232 (0.65), 1.600 (10.90), 1.617 (10.97), 2.007 (0.50), 2.020 (1.21), 2.036 (2.39), 2.055 (2.56), 2.074 (1.19), 2.086 (0.62), 2.529 (2.74), 3.051 (0.92), 3.069 (2.90), 3.083 (3.37), 3.086 (3.34), 3.100 (2.85), 3.118 (0.85), 3.373 (2.35), 3.384 (1.70), 3.398 (4.37), 3.410 (2.15), 3.427 (0.94), 3.445 (4.05), 3.470 (3.43), 3.487 (1.08), 3.494 (0.86), 3.512 (0.50), 4.136 (2.58), 4.153 (5.19), 4.171 (2.59), 6.166 (0.83), 6.183 (3.03), 6.199 (3.56), 6.207 (2.64), 6.215 (1.53), 6.221 (1.44), 6.234 (11.93), 6.244 (0.97), 6.344 (6.99), 7.231 (1.30), 7.249 (3.21), 7.264 (1.36), 7.268 (2.19), 7.333 (3.94), 7.352 (6.57), 7.367 (1.23), 7.371 (3.19), 7.508 (5.99), 7.525 (5.01), 7.528 (3.83), 7.904 (13.64).

### Example 914

### 5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyrazin-2-amine (Diastereomer 1 and Diastereomer 2)

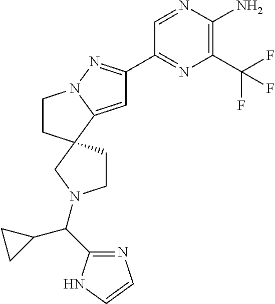 [SMILES: Nc1ncc(-c2cc3n(n2)CC[C@@]32CCN(C(c3ncc[nH]3)C3CC3)C2)nc1C(F)(F)F]

5-[(3R)-5′,6′-Dihydrospiro[pyrrolidine-3,4′-pyrrolo[1,2-b]pyrazol]-2′-yl]-3-(trifluoromethyl)pyrazin-2-amine (64.0 mg, 197 μmol, Intermediate 535) and cyclopropyl(1H-imidazol-2-yl)methanone (40.3 mg, 296 μmol) were dissolved in methanol (3.4 mL). N,N-Diisopropylethylamine (140 μL, 0.79 mmol) and titanium(IV) isopropoxide (170 μL, 590 μmol) were added and stirred for 3 h at 60° C. To the reaction mixture allowed to cool down, sodium cyanoborohydride (1.2 mL, 0.5 M, 0.59 mmol) was added and stirred for 16 h at 60° C. The reaction mixture was allowed to cool down, diluted with water and stirred for 15 min. The suspension was filtered and washed with methanol. The filtrate was concentrated. The residue was purified by preparative HPLC to give 13.0 mg (92% purity, 14% yield) of the title compound.

LC-MS (Method 1): Rₜ=0.94 min; MS (ESIpos): m/z=446 [M+H]⁺

¹H-NMR (400 MHz, DMSO-d6) δ [ppm]: −0.023 (0.66), 0.063 (1.78), 0.075 (2.39), 0.087 (1.67), 0.100 (0.57), 0.279 (0.66), 0.302 (1.94), 0.315 (2.53), 0.321 (3.19), 0.338 (4.03), 0.350 (2.45), 0.362 (2.03), 0.374 (1.41), 0.384 (0.80), 0.597 (1.11), 0.618 (1.85), 0.628 (2.00), 0.640 (1.43), 0.851 (0.70), 1.188 (0.55), 1.201 (1.09), 1.212 (1.46), 1.224 (2.81), 1.232 (4.39), 1.263 (1.37), 1.276 (0.73), 1.751 (0.40), 1.904 (0.59), 1.911 (0.53), 1.930 (0.88), 1.944 (1.36), 1.957 (1.85), 1.968 (1.57), 1.986 (2.09), 2.001 (2.14), 2.014 (2.40), 2.020 (2.52), 2.034 (2.57), 2.046 (1.27), 2.052 (1.20), 2.066 (0.98), 2.291 (0.66), 2.318 (0.77), 2.323 (1.60), 2.327 (2.24), 2.331 (1.64), 2.337 (0.77), 2.469 (1.88), 2.518 (11.30), 2.523 (6.59), 2.565 (5.74), 2.576 (4.89), 2.585 (3.48), 2.607 (3.16), 2.624 (2.10), 2.644 (4.05), 2.668 (4.97), 2.675 (4.16), 2.699 (3.20), 2.748 (0.77), 2.771 (4.16), 2.786 (1.56), 2.794 (2.96), 2.807 (0.68), 2.895 (0.97), 2.909 (0.95), 2.916 (1.17), 2.931 (1.27), 2.942 (3.36), 2.965 (2.76), 3.159 (0.67), 3.171 (0.68), 4.077 (0.63), 4.085 (1.33), 4.104 (2.82), 4.119 (2.87), 4.127 (3.84), 4.144 (6.63), 4.162 (3.12), 6.386 (14.87), 6.393 (16.00), 6.437 (0.74), 6.749 (3.24), 6.898 (11.83), 7.012 (2.88), 8.716 (10.78), 11.817 (2.79).

### Example 915

### 5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyrazin-2-amine (Diastereomer 1)

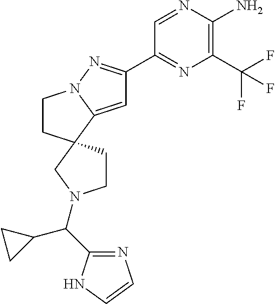 [SMILES: Nc1ncc(-c2cc3n(n2)CC[C@@]32CCN(C(c3ncc[nH]3)C3CC3)C2)nc1C(F)(F)F]

The compound 5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyrazin-2-amine (diastereomer 1 and diastereomer 2, Example 914) was separated into diastereomers by preparative chiral HPLC to give 27.0 mg (92% purity, 18% yield) of the title compound (diastereomer 1 Rₜ=6.1-7.3 min).

Preparative Chiral HPLC Method:

Instrument: Sepiatec: Prep SFC100; Column: Chiral Art Cellulose SB 5μ, 250×50; eluent A: CO2; eluent B: 2-propanol+0.4 vol % diethylamine; isocratic: 20% B; flow: 100 mL/min; temperature: 40° C.; BPR: 150 bar UV: 272 nm

Analytical Chiral HPLC Method:

Instrument: Waters Acquity UPC2 QDA; Column: Chiral Art Cellulose SB 3μ, 100×4.6; eluent A: CO2; eluent B: 2-propanol+0.4 vol % diethylamine; isocratic: 30% B; flow: 4 mL/min; temperature: 40° C.; BPR: 1800 psi UV: 280 nm

Analytical chiral HPLC Rₜ=1.19 min

LC-MS (Method 1): Rₜ=0.94 min; MS (ESIpos): m/z=445 [M+H]⁺

¹H-NMR (400 MHz, DMSO-d6) δ [ppm]: 0.011 (0.59), 0.023 (1.08), 0.037 (1.23), 0.044 (0.80), 0.049 (0.69), 0.255 (0.50), 0.265 (0.82), 0.277 (1.16), 0.282 (2.10), 0.300 (2.54), 0.313 (1.12), 0.324 (0.69), 0.556 (0.66), 0.574 (0.98), 0.581 (0.83), 0.587 (0.93), 0.602 (0.52), 0.946 (0.89), 0.962 (0.98), 0.989 (14.51), 1.004 (15.14), 1.163 (0.47), 1.174 (0.62), 1.186 (1.15), 1.194 (1.66), 1.207 (1.21), 1.218 (0.65), 1.225 (0.62), 1.892 (0.59), 1.905 (0.98), 1.921 (1.22), 1.939 (0.76), 1.961 (0.72), 1.976 (0.96), 1.982 (1.10), 1.996 (1.00), 2.008 (0.59), 2.013 (0.53), 2.028 (0.42), 2.285 (0.56), 2.289 (0.82), 2.294 (0.59), 2.431 (0.77), 2.481 (4.26), 2.485 (2.37), 2.498 (1.19), 2.516 (3.57), 2.569 (1.84), 2.586 (1.26), 2.601 (1.61), 2.607 (3.29), 2.619 (0.73), 2.622 (0.67), 2.631 (3.70), 2.710 (0.60), 2.732 (1.29), 2.748 (1.09), 2.754 (0.72), 2.769 (0.47), 2.904 (2.55), 2.927 (2.24), 4.089 (2.48), 4.106 (5.09), 4.124 (2.43), 4.310 (0.57), 4.320 (0.57), 6.349 (0.44), 6.356 (16.00), 6.711 (1.25), 6.861 (5.93), 6.971 (1.21), 8.678 (6.01), 8.680 (5.84), 11.779 (1.00).

### Example 916

### 5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyrazin-2-amine (Diastereomer 2)

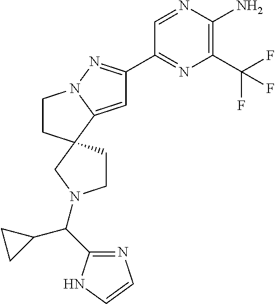 [SMILES: Nc1ncc(-c2cc3n(n2)CC[C@@]32CCN(C(c3ncc[nH]3)C3CC3)C2)nc1C(F)(F)F]

The diastereomers were separated by preparative chiral HPLC (method see Example 915) to give 22.0 mg (92% purity, 15% yield) of the title compound (diastereomer 2 Rₜ=7.6-10.0 min).

Analytical chiral HPLC (method see Example 915): Rₜ=1.92 min

LC-MS (Method 1): Rₜ=0.94 min; MS (ESIpos): m/z=445 [M+H]⁺

¹H-NMR (400 MHz, DMSO-d6) δ [ppm]: 0.011 (0.81), 0.023 (1.22), 0.035 (1.35), 0.047 (1.05), 0.059 (0.45), 0.253 (0.60), 0.262 (0.96), 0.274 (1.20), 0.284 (1.41), 0.296 (1.29), 0.307 (1.41), 0.320 (1.44), 0.333 (1.29), 0.343 (0.84), 0.554 (0.47), 0.564 (0.69), 0.576 (1.05), 0.586 (1.14), 0.598 (0.94), 0.609 (0.56), 0.809 (0.39), 0.822 (0.43), 0.942 (1.37), 0.958 (1.37), 0.985 (11.86), 1.001 (12.55), 1.010 (0.67), 1.028 (0.43), 1.160 (0.58), 1.171 (0.79), 1.181 (1.42), 1.191 (2.19), 1.203 (1.69), 1.221 (0.84), 1.913 (0.75), 1.918 (0.69), 1.927 (1.16), 1.944 (1.87), 1.961 (1.63), 1.973 (1.35), 1.979 (1.41), 1.992 (1.42), 2.004 (0.56), 2.011 (0.52), 2.024 (0.41), 2.280 (0.75), 2.286 (1.05), 2.290 (0.77), 2.426 (0.96), 2.476 (5.70), 2.481 (3.54), 2.500 (3.15), 2.510 (1.57), 2.524 (4.95), 2.561 (2.06), 2.576 (0.82), 2.618 (0.54), 2.623 (0.97), 2.627 (1.52), 2.635 (4.01), 2.659 (3.48), 2.730 (3.35), 2.754 (2.75), 2.854 (0.67), 2.867 (0.96), 2.874 (1.24), 2.890 (1.22), 2.909 (0.58), 4.032 (0.54), 4.035 (0.64), 4.044 (1.42), 4.051 (0.75), 4.063 (3.04), 4.077 (2.68), 4.089 (0.84), 4.097 (1.42), 4.104 (0.81), 4.110 (0.60), 4.124 (0.43), 4.305 (0.82), 4.315 (0.81), 6.344 (16.00), 6.352 (0.81), 6.709 (1.48), 6.853 (7.19), 6.974 (1.42), 8.671 (6.99), 11.777 (1.22).

### Example 917

### 5-{(3R)-1-[1-(1H-pyrazol-3-yl)propyl]-5′,6′-dihydrospiro[pyrrolidine-3,4′-pyrrolo[1,2-b]pyrazol]-2′-yl}-3-(trifluoromethyl)pyridin-2-amine (Diastereomer 1)

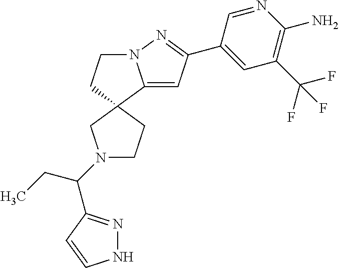 [SMILES: CCC(c1cc[nH]n1)N1CC[C@@]2(CCn3nc(-c4cnc(N)c(C(F)(F)F)c4)cc32)C1]

The compound 5-{(3R)-1-[1-(1H-pyrazol-3-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2, Example 900) was separated into diastereomers by preparative chiral HPLC to give 37.0 mg (99% purity) of the title compound (diastereomer 1 Rₜ=18.15-23.58 min).

Preparative Chiral HPLC Method:

Instrument: PrepCon Labomatic HPLC-2; Column: YMC Cellulose SB 5μ, 250×50; eluent A: hexane+0.1 vol % diethylamine; eluent B: ethanol; isocratic: 30% B; flow: 90 mL/min; temperature: 25° C.; UV: 280 nm

Analytical Chiral HPLC Method:

Instrument: Thermo Fisher UltiMate 3000; Column: YMC Cellulose SA 3μ, 100×4.6; eluent A: hexane+0.1 vol % diethylamine; eluent B: ethanol; isocratic: 30% B; flow: 1.4 mL/min; temperature: 25° C.; UV: 280 nm

Analytical chiral HPLC Rₜ=2.71 min

LC-MS (Method 1): Rₜ=1.02 min; MS (ESIpos): m/z=432 [M+H]⁺

¹H-NMR (400 MHz, CHLOROFORM-d) δ [ppm]: −0.125 (0.75), 0.014 (7.26), 0.033 (7.24), 0.042 (1.47), 0.170 (0.73), 0.776 (7.06), 0.784 (1.52), 0.794 (16.00), 0.813 (7.59), 0.820 (1.30), 0.825 (0.68), 0.832 (0.70), 0.839 (1.41), 0.850 (0.68), 0.854 (0.66), 0.858 (0.81), 0.864 (0.44), 0.871 (0.62), 0.881 (0.59), 1.101 (0.97), 1.125 (0.90), 1.167 (0.70), 1.175 (2.13), 1.182 (0.46), 1.192 (0.46), 1.210 (1.80), 1.318 (0.97), 1.356 (3.32), 1.517 (0.48), 1.535 (0.42), 1.679 (0.79), 1.697 (0.92), 1.701 (0.92), 1.713 (1.17), 1.731 (1.14), 1.734 (1.19), 1.753 (0.95), 1.845 (1.01), 1.857 (1.19), 1.863 (1.14), 1.876 (1.34), 1.890 (1.01), 1.898 (1.39), 1.914 (1.21), 1.918 (1.14), 1.931 (1.50), 1.946 (1.47), 1.951 (1.34), 1.966 (1.17), 2.041 (0.57), 2.061 (1.21), 2.075 (1.47), 2.081 (1.63), 2.096 (1.65), 2.107 (1.14), 2.113 (1.10), 2.128 (0.97), 2.456 (0.88), 2.472 (1.01), 2.476 (1.19), 2.488 (1.80), 2.504 (1.83), 2.508 (1.96), 2.524 (1.58), 2.568 (1.36), 2.582 (1.78), 2.586 (1.63), 2.601 (1.87), 2.634 (0.86), 2.653 (0.70), 2.675 (1.54), 2.686 (2.60), 2.709 (4.56), 2.739 (3.39), 2.763 (1.80), 2.788 (1.54), 2.804 (1.39), 2.826 (0.62), 3.474 (1.45), 3.486 (1.63), 3.496 (1.61), 3.508 (1.39), 4.070 (0.57), 4.085 (0.79), 4.089 (0.68), 4.097 (2.33), 4.105 (0.90), 4.113 (4.80), 4.116 (3.26), 4.127 (3.04), 4.132 (4.53), 4.140 (0.95), 4.148 (2.29), 4.155 (0.68), 4.160 (0.79), 4.175 (0.57), 4.970 (6.40), 6.110 (10.41), 6.162 (5.37), 6.167 (5.35), 7.491 (6.29), 7.496 (6.16), 8.057 (4.42), 8.061 (4.38), 8.553 (4.20), 8.557 (4.20).

### Example 918

### 5-{(3R)-1-[1-(1H-pyrazol-3-yl)propyl]-5′,6′-dihydrospiro[pyrrolidine-3,4′-pyrrolo[1,2-b]pyrazol]-2′-yl}-3-(trifluoromethyl)pyridin-2-amine (Diastereomer 2)

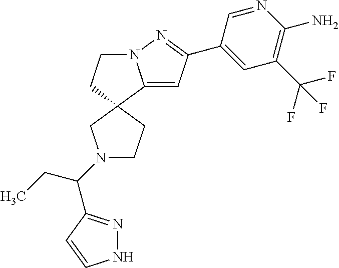 [SMILES: CCC(c1cc[nH]n1)N1CC[C@@]2(CCn3nc(-c4cnc(N)c(C(F)(F)F)c4)cc32)C1]

For the preparation of the diastereomeric mixture of the title compound see Example 900. The diastereomers were separated by preparative chiral HPLC (method see Example 917 Example 915

### 5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyrazin-2-amine (Diastereomer 1)

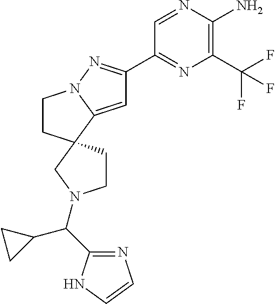 [SMILES: Nc1ncc(-c2cc3n(n2)CC[C@@]32CCN(C(c3ncc[nH]3)C3CC3)C2)nc1C(F)(F)F]

The compound 5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyrazin-2-amine (diastereomer 1 and diastereomer 2, Example 914) was separated into diastereomers by preparative chiral HPLC to give 27.0 mg (92% purity, 18% yield) of the title compound (diastereomer 1 Rt=6.1-7.3 min).

Preparative Chiral HPLC Method:

Instrument: Sepiatec: Prep SFC100; Column: Chiral Art Cellulose SB 5μ, 250×50; eluent A: CO2; eluent B: 2-propanol+0.4 vol % diethylamine; isocratic: 20% B; flow: 100 mL/min; temperature: 40° C.; BPR: 150 bar UV: 272 nm

Analytical Chiral HPLC Method:

Instrument: Waters Acquity UPC2 QDA; Column: Chiral Art Cellulose SB 3μ, 100×4.6; eluent A: CO2; eluent B: 2-propanol+0.4 vol % diethylamine; isocratic: 30% B; flow: 4 mL/min; temperature: 40° C.; BPR: 1800 psi UV: 280 nm

Analytical chiral HPLC Rt=1.19 min

LC-MS (Method 1): Rt=0.94 min; MS (ESIpos): m/z=445 [M+H]⁺

1H-NMR (400 MHz, DMSO-d6) δ [ppm]: 0.011 (0.59), 0.023 (1.08), 0.037 (1.23), 0.044 (0.80), 0.049 (0.69), 0.255 (0.50), 0.265 (0.82), 0.277 (1.16), 0.282 (2.10), 0.300 (2.54), 0.313 (1.12), 0.324 (0.69), 0.556 (0.66), 0.574 (0.98), 0.581 (0.83), 0.587 (0.93), 0.602 (0.52), 0.946 (0.89), 0.962 (0.98), 0.989 (14.51), 1.004 (15.14), 1.163 (0.47), 1.174 (0.62), 1.186 (1.15), 1.194 (1.66), 1.207 (1.21), 1.218 (0.65), 1.225 (0.62), 1.892 (0.59), 1.905 (0.98), 1.921 (1.22), 1.939 (0.76), 1.961 (0.72), 1.976 (0.96), 1.982 (1.10), 1.996 (1.00), 2.008 (0.59), 2.013 (0.53), 2.028 (0.42), 2.285 (0.56), 2.289 (0.82), 2.294 (0.59), 2.431 (0.77), 2.481 (4.26), 2.485 (2.37), 2.498 (1.19), 2.516 (3.57), 2.569 (1.84), 2.586 (1.26), 2.601 (1.61), 2.607 (3.29), 2.619 (0.73), 2.622 (0.67), 2.631 (3.70), 2.710 (0.60), 2.732 (1.29), 2.748 (1.09), 2.754 (0.72), 2.769 (0.47), 2.904 (2.55), 2.927 (2.24), 4.089 (2.48), 4.106 (5.09), 4.124 (2.43), 4.310 (0.57), 4.320 (0.57), 6.349 (0.44), 6.356 (16.00), 6.711 (1.25), 6.861 (5.93), 6.971 (1.21), 8.678 (6.01), 8.680 (5.84), 11.779 (1.00).

### Example 916

### 5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyrazin-2-amine (Diastereomer 2)

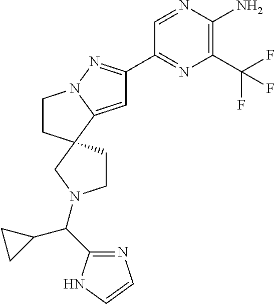 [SMILES: Nc1ncc(-c2cc3n(n2)CC[C@@]32CCN(C(c3ncc[nH]3)C3CC3)C2)nc1C(F)(F)F]

The diastereomers were separated by preparative chiral HPLC (method see Example 915) to give 22.0 mg (92% purity, 15% yield) of the title compound (diastereomer 2 Rt=7.6-10.0 min).

Analytical chiral HPLC (method see Example 915): Rt=1.92 min

LC-MS (Method 1): Rt=0.94 min; MS (ESIpos): m/z=445 [M+H]⁺

1H-NMR (400 MHz, DMSO-d6) δ [ppm]: 0.011 (0.81), 0.023 (1.22), 0.035 (1.35), 0.047 (1.05), 0.059 (0.45), 0.253 (0.60), 0.262 (0.96), 0.274 (1.20), 0.284 (1.41), 0.296 (1.29), 0.307 (1.41), 0.320 (1.44), 0.333 (1.29), 0.343 (0.84), 0.554 (0.47), 0.564 (0.69), 0.576 (1.05), 0.586 (1.14), 0.598 (0.94), 0.609 (0.56), 0.809 (0.39), 0.822 (0.43), 0.942 (1.37), 0.958 (1.37), 0.985 (11.86), 1.001 (12.55), 1.010 (0.67), 1.028 (0.43), 1.160 (0.58), 1.171 (0.79), 1.181 (1.42), 1.191 (2.19), 1.203 (1.69), 1.221 (0.84), 1.913 (0.75), 1.918 (0.69), 1.927 (1.16), 1.944 (1.87), 1.961 (1.63), 1.973 (1.35), 1.979 (1.41), 1.992 (1.42), 2.004 (0.56), 2.011 (0.52), 2.024 (0.41), 2.280 (0.75), 2.286 (1.05), 2.290 (0.77), 2.426 (0.96), 2.476 (5.70), 2.481 (3.54), 2.500 (3.15), 2.510 (1.57), 2.524 (4.95), 2.561 (2.06), 2.576 (0.82), 2.618 (0.54), 2.623 (0.97), 2.627 (1.52), 2.635 (4.01), 2.659 (3.48), 2.730 (3.35), 2.754 (2.75), 2.854 (0.67), 2.867 (0.96), 2.874 (1.24), 2.890 (1.22), 2.909 (0.58), 4.032 (0.54), 4.035 (0.64), 4.044 (1.42), 4.051 (0.75), 4.063 (3.04), 4.077 (2.68), 4.089 (0.84), 4.097 (1.42), 4.104 (0.81), 4.110 (0.60), 4.124 (0.43), 4.305 (0.82), 4.315 (0.81), 6.344 (16.00), 6.352 (0.81), 6.709 (1.48), 6.853 (7.19), 6.974 (1.42), 8.671 (6.99), 11.777 (1.22).

Example 917) to give 40.0 mg (99% purity) of the title compound (diastereomer 2 Rₜ=25.51-31.68 min).

Analytical chiral HPLC (method see Example 917): Rₜ=4.06 min

LC-MS (Method 1): Rₜ=1.02 min; MS (ESIpos): m/z=432 [M+H]⁺

¹H-NMR (400 MHz, CHLOROFORM-d) δ [ppm]: −0.125 (1.19), 0.015 (10.90), 0.033 (11.49), 0.042 (2.47), 0.170 (1.15), 0.777 (6.95), 0.796 (16.00), 0.814 (7.57), 0.825 (0.56), 0.832 (0.59), 0.839 (1.15), 0.850 (0.56), 0.854 (0.53), 0.858 (0.66), 0.871 (0.53), 0.882 (0.53), 1.101 (0.82), 1.125 (0.72), 1.167 (0.63), 1.175 (1.78), 1.210 (1.09), 1.318 (1.02), 1.356 (3.36), 1.516 (0.43), 1.679 (0.79), 1.697 (0.92), 1.702 (0.89), 1.713 (1.09), 1.720 (0.89), 1.731 (1.05), 1.735 (1.12), 1.754 (0.89), 1.849 (0.40), 1.867 (0.99), 1.880 (1.09), 1.885 (1.09), 1.898 (1.25), 1.913 (0.79), 1.918 (0.79), 1.931 (0.72), 1.937 (0.89), 1.956 (1.09), 1.969 (1.25), 1.987 (1.65), 2.006 (1.09), 2.047 (0.69), 2.062 (1.22), 2.075 (1.45), 2.083 (1.58), 2.095 (2.07), 2.106 (1.02), 2.115 (0.95), 2.127 (0.89), 2.458 (0.76), 2.473 (0.89), 2.478 (0.99), 2.490 (1.65), 2.505 (1.74), 2.510 (1.84), 2.526 (1.48), 2.553 (1.38), 2.567 (1.68), 2.573 (1.61), 2.586 (1.91), 2.599 (0.95), 2.641 (2.30), 2.663 (5.07), 2.674 (4.31), 2.698 (0.95), 2.896 (0.69), 2.909 (0.92), 2.917 (1.25), 2.929 (1.19), 2.939 (0.76), 2.951 (0.63), 3.463 (1.25), 3.475 (1.42), 3.486 (1.35), 3.498 (1.22), 4.044 (0.76), 4.059 (0.89), 4.063 (0.99), 4.071 (2.11), 4.079 (0.99), 4.086 (2.24), 4.091 (2.40), 4.101 (2.17), 4.106 (2.07), 4.115 (2.37), 4.122 (2.21), 4.129 (1.05), 4.135 (2.04), 4.142 (0.99), 4.149 (0.92), 4.163 (0.79), 4.972 (5.73), 6.069 (8.59), 6.161 (5.20), 6.166 (5.20), 7.497 (6.06), 7.502 (5.93), 8.049 (4.12), 8.054 (4.18), 8.543 (4.02), 8.547 (3.95).

### Example 919

### 5-{(3R)-1-[cyclopropyl(1H-1,2,3-triazol-5-yl)methyl]-5′,6′-dihydrospiro[pyrrolidine-3,4′-pyrrolo[1,2-b]pyrazol]-2′-yl}-3-(trifluoromethyl)pyridin-2-amine (Diastereomer 1 and Diastereomer 2)

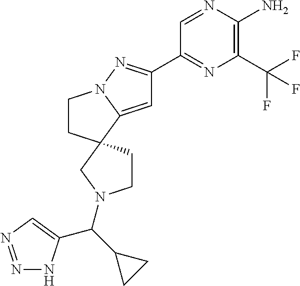 [SMILES: Nc1ncc(-c2cc3n(n2)CC[C@@]32CCN(C(c3cnn[nH]3)C3CC3)C2)nc1C(F)(F)F]

5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (414 mg, 93% purity, 1.07 mmol) and cyclopropyl(1H-1,2,3-triazol-5-yl)methanone (220 mg, 1.61 mmol) were dissolved in methanol (11 mL). N,N-Diisopropylethylamine (930 μL, 5.4 mmol) and titanium(IV) isopropoxide (950 μL, 3.2 mmol) were added and stirred over night at 60° C. The reaction mixture was allowed to cool down, sodium cyanoborohydride (168 mg, 2.68 mmol) was added and stirred for 2 h at 60° C., for 2 h at 80° C. and overnight at room temperature. The reaction mixture was diluted with water and stirred for 10 min. The suspension was filtered and the residue was washed with methanol. The filtrate was concentrated. The aqueous residue was extracted with ethyl acetate. The combined organic layers were washed with brine, dried and concentrated. The crude product was purified by preparative HPLC to give 176 mg (95% purity, 35% yield) of the title compound.

LC-MS (Method 1): Rₜ=0.73 min; MS (ESIpos): m/z=445 [M+H]⁺

¹H-NMR (400 MHz, DMSO-d6) δ [ppm]: −0.099 (0.64), 0.291 (2.28), 0.303 (2.81), 0.322 (2.42), 0.339 (1.64), 0.351 (1.81), 0.362 (1.64), 0.373 (1.42), 0.598 (2.10), 0.801 (0.53), 1.163 (2.13), 1.181 (2.95), 1.892 (0.71), 1.907 (1.56), 1.932 (3.31), 1.941 (4.94), 1.963 (3.02), 2.272 (1.42), 2.277 (2.03), 2.281 (1.49), 2.398 (1.32), 2.415 (2.77), 2.467 (10.63), 2.472 (7.18), 2.489 (2.38), 2.516 (2.52), 2.567 (3.09), 2.596 (3.38), 2.619 (5.65), 2.648 (3.56), 2.671 (1.67), 2.729 (1.35), 2.762 (1.78), 2.787 (2.60), 2.825 (2.49), 2.847 (1.96), 2.878 (1.32), 4.003 (2.03), 4.021 (3.59), 4.032 (5.23), 4.049 (7.40), 4.067 (2.99), 6.367 (3.88), 6.386 (6.36), 6.416 (0.43), 6.465 (16.00), 7.702 (0.71), 7.956 (6.04), 7.961 (9.64), 7.967 (4.98), 8.542 (9.14).

### Example 920

### 5-{(3R)-1-[cyclopropyl(1H-1,2,4-triazol-5-yl)methyl]-5′,6′-dihydrospiro[pyrrolidine-3,4′-pyrrolo[1,2-b]pyrazol]-2′-yl}-3-[(1R)-1-(2,6-difluorophenyl)ethoxy]pyridin-2-amine (Diastereomer 1 and Diastereomer 2)

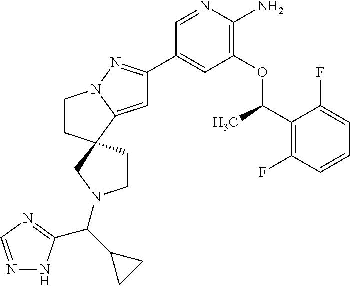 [SMILES: C[C@@H](Oc1cc(-c2cc3n(n2)CC[C@@]32CCN(C(c3ncn[nH]3)C3CC3)C2)cnc1N)c1c(F)cccc1F]

3-[(1R)-1-(2,6-Difluorophenyl)ethoxy]-5-[(3R)-5′,6′-dihydrospiro[pyrrolidine-3,4′-pyrrolo[1,2-b]pyrazol]-2′-yl]pyridin-2-amine hydrogen chloride (1/1) (120 mg, 268 μmol) and cyclopropyl(1H-1,2,4-triazol-5-yl)methanone (55.1 mg, 402 μmol) were dissolved in methanol (2.8 mL). N,N-Diisopropylethylamine (140 μL, 800 μmol) and titanium(IV) isopropoxide (240 μL, 800 μmol) were added and stirred for 1.5 h at 60° C. The reaction mixture was allowed to cool down, sodium cyanoborohydride (50.5 mg, 804 μmol) was added and stirred for 16 h at 60° C. The reaction mixture was allowed to cool down, water (0.5 mL) was added and stirred for 15 min. The suspension was filtered and the filter cake was washed with methanol. The filtrate was concentrated. The residue was purified by preparative HPLC to give 50.0 mg (94% purity, 33% yield) of the title compound.

LC-MS (Method 1): Rₜ=0.94 min; MS (ESIpos): m/z=533 [M+H]⁺

¹H NMR (DMSO-d₆, 400 MHz): δ (ppm) 8.04 (s, 1H), 7.89 (d, 1H), 7.35-7.44 (m, 1H), 7.31-7.34 (m, 1H), 7.06-7.15 (m, 2H), 6.17-6.21 (m, 1H), 5.79-5.86 (m, 1H), 5.64 (s, 2H), 3.98-4.10 (m, 2H), 2.92-3.00 (m, 1H), 2.83-2.90 (m, 1H), 2.74-2.81 (m, 1H), 2.54-2.66 (m, 3H), 2.39-2.46 (m, 1H), 1.87-2.03 (m, 2H), 1.75 (d, 3H), 1.21-1.32 (m, 1H), 0.58-0.68 (m, 1H), 0.29-0.43 (m, 2H), 0.04-0.13 (m, 1H).

### Example 921

### 5-{(3R)-1-[(1-methylcyclobutyl)(4H-1,2,4-triazol-3-yl)methyl]-5′,6′-dihydrospiro[pyrrolidine-3,4′-pyrrolo[1,2-b]pyrazol]-2′-yl}-3-(trifluoromethyl)pyridin-2-amine (Diastereomer 1 and Diastereomer 2)

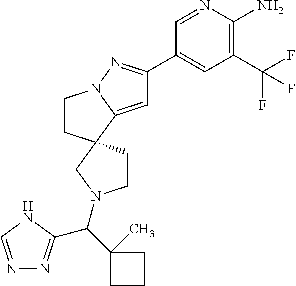 [SMILES: CC1(C(c2nnc[nH]2)N2CC[C@@]3(CCn4nc(-c5cnc(N)c(C(F)(F)F)c5)cc43)C2)CCC1]

5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (97.3 mg, 270 μmol) and (1-methylcyclobutyl)(4H-1,2,4-triazol-3-yl)methanone (67.0 mg, 406 μmol, Intermediate 572) were dissolved in methanol (5.8 mL). N,N-Diisopropylethylamine (190 μL, 1.1 mmol) and titanium(IV) isopropoxide (240 μL, 810 μmol) were added and stirred for 3 h at 60° C. The reaction mixture was allowed to cool down, sodium cyanoborohydride (51.0 mg, 811 μmol) was added and the mixture was stirred over the weekend at 60° C. The reaction mixture was allowed to cool down, filtered and the filtrate was diluted with ethyl acetate. The organic phase was washed with saturated sodium bicarbonate solution, water and brine, dried and concentrated. The residue was purified by column chromatography (silica gel NH2, hexane/ethyl acetate (0-100%) and ethyl acetate/ethanol (0-100%) gradient) followed by preparative HPLC to give 22.6 mg (90% purity, 16% yield) of the title compound.

LC-MS (Method 1): Rₜ=1.05 min; MS (ESIpos): m/z=473 [M+H]⁺

¹H-NMR (400 MHz, DMSO-d6) δ [ppm]: 1.195 (0.66), 1.223 (3.99), 1.241 (8.28), 1.258 (4.30), 1.300 (0.61), 1.401 (3.51), 1.429 (4.30), 1.671 (0.48), 1.693 (0.57), 1.789 (0.53), 1.919 (0.44), 1.942 (0.83), 1.963 (0.88), 1.980 (1.01), 1.999 (1.49), 2.018 (1.10), 2.040 (0.66), 2.056 (16.00), 2.064 (0.61), 2.092 (0.88), 2.117 (1.23), 2.142 (1.18), 2.166 (0.66), 2.357 (0.92), 2.386 (0.83), 2.391 (1.84), 2.395 (2.63), 2.400 (1.84), 2.405 (0.79), 2.511 (1.49), 2.586 (10.26), 2.591 (7.98), 2.604 (1.36), 2.620 (1.88), 2.636 (1.23), 2.653 (0.88), 2.672 (0.57), 2.728 (1.18), 2.733 (2.19), 2.737 (2.98), 2.742 (2.41), 2.747 (2.24), 2.769 (1.01), 2.852 (0.75), 2.875 (0.66), 3.669 (2.06), 3.692 (2.28), 4.068 (1.10), 4.085 (3.38), 4.103 (3.33), 4.121 (1.27), 4.132 (0.66), 4.143 (0.88), 4.151 (0.96), 4.158 (1.40), 4.167 (0.92), 4.175 (1.71), 4.193 (0.83), 4.822 (0.48), 6.507 (3.33), 6.521 (0.66), 6.535 (0.88), 6.591 (4.08), 7.447 (0.39), 7.463 (0.44), 7.486 (0.66), 8.066 (2.41), 8.646 (2.32).

### Example 922

### 5-{(3R)-1-[1-(4-chloro-1H-imidazol-2-yl)propyl]-5′,6′-dihydrospiro[pyrrolidine-3,4′-pyrrolo[1,2-b]pyrazol]-2′-yl}-3-(trifluoromethyl)pyridin-2-amine (Diastereomer 1)

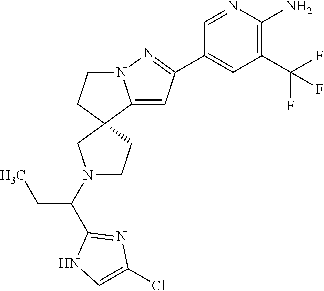 [SMILES: CCC(c1nc(Cl)c[nH]1)N1CC[C@@]2(CCn3nc(-c4cnc(N)c(C(F)(F)F)c4)cc32)C1]

The compound 5-{(3R)-1-[1-(4-chloro-1H-imidazol-2-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2, Example 909) was separated into diastereomers by preparative chiral HPLC to give 15.0 mg (98% purity, 37% yield) of the title compound (diastereomer 1 Rₜ=13.8-16.8 min).

Preparative Chiral HPLC Method:

Instrument: Sepiatec: Prep SFC100; Column: Chiral Art Amylose SA 5μ, 250×50; eluent A: CO2; eluent B: methanol+0.2 vol % aqueous ammonia (32%); isocratic: 10% B; flow: 100 mL/min; temperature: 40° C.; BPR: 150 bar; UV: 267 nm

Analytical Chiral HPLC Method:

Instrument: Waters acquity UPC2 QDA; Column: Chiral Art Amylose SA 3μ, 100×4.6; eluent A: CO2; eluent B: methanol+0.2 vol % aqueous ammonia (32%); isocratic: 10% B; flow: 4 mL/min; temperature: 40° C.; BPR: 1800 psi; UV: 280 nm

Analytical chiral HPLC Rₜ=3.96 min

Specific Rotation: 150.13

LC-MS (Method 1): Rₜ=1.04 min; MS (ESIpos): m/z=466 [M+H]⁺

¹H-NMR (400 MHz, CHLOROFORM-d) δ [ppm]: 0.830 (7.06), 0.848 (16.00), 0.867 (7.58), 1.047 (0.41), 1.265 (0.85), 1.426 (0.57), 1.631 (11.10), 1.727 (0.75), 1.746 (1.07), 1.763 (1.20), 1.781 (1.25), 1.799 (0.81), 1.931 (1.07), 1.941 (1.24), 1.949 (1.26), 1.960 (1.41), 1.976 (1.05), 1.983 (1.00), 1.994 (1.50), 2.009 (1.10), 2.014 (1.22), 2.027 (1.62), 2.041 (1.41), 2.047 (1.36), 2.061 (1.12), 2.173 (1.10), 2.188 (1.62), 2.194 (1.52), 2.209 (1.57), 2.221 (1.11), 2.226 (1.04), 2.242 (0.85), 2.548 (0.70), 2.568 (1.01), 2.580 (1.79), 2.596 (2.04), 2.600 (2.17), 2.628 (1.46), 2.642 (1.87), 2.647 (1.82), 2.661 (2.00), 2.674 (0.90), 2.679 (0.89), 2.696 (1.19), 2.711 (3.17), 2.734 (5.20), 2.756 (0.91), 2.797 (3.38), 2.820 (2.04), 2.905 (0.96), 2.927 (1.63), 2.941 (1.52), 2.964 (0.69), 3.516 (1.61), 3.526 (1.82), 3.537 (1.77), 3.548 (1.53), 4.138 (0.64), 4.157 (0.96), 4.166 (2.17), 4.174 (1.00), 4.185 (3.35), 4.202 (4.14), 4.209 (2.48), 4.216 (1.13), 4.223 (2.18), 4.230 (0.86), 4.236 (0.79), 4.250 (0.62), 5.011 (7.75), 5.310 (11.88), 6.207 (11.33), 6.887 (6.56), 7.007 (0.41), 7.529 (0.41), 8.116 (5.03), 8.120 (5.09), 8.612 (4.84), 8.616 (4.78), 9.193 (0.95).

### Example 923

### 5-{(3R)-1-[1-(4-chloro-1H-imidazol-2-yl)propyl]-5′,6′-dihydrospiro[pyrrolidine-3,4′-pyrrolo[1,2-b]pyrazol]-2′-yl}-3-(trifluoromethyl)pyridin-2-amine (Diastereomer 2)

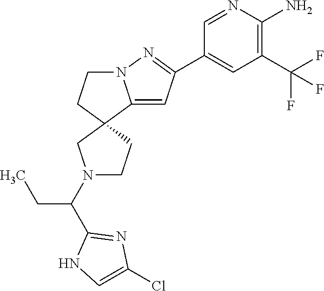 [SMILES: CCC(c1nc(Cl)c[nH]1)N1CC[C@@]2(CCn3nc(-c4cnc(N)c(C(F)(F)F)c4)cc32)C1]

For the preparation of the diastereomeric mixture of the title compound see Example 909. The diastereomers were separated by preparative chiral HPLC (method see Example 922) to give 13.7 mg (98% purity, 34% yield) of the title compound (diastereomer 2 Rₜ=17.1-20.0 min).

Analytical chiral HPLC (method see Example 922): Rₜ=4.97 min

Specific Rotation: 36.99

LC-MS (Method 1): Rₜ=1.04 min; MS (ESIpos): m/z=466 [M+H]⁺

¹H-NMR (400 MHz, CHLOROFORM-d) δ [ppm]: 0.841 (7.15), 0.859 (16.00), 0.877 (7.65), 1.265 (0.80), 1.634 (12.31), 1.718 (0.42), 1.736 (0.78), 1.754 (1.09), 1.770 (1.29), 1.791 (1.34), 1.810 (0.92), 1.932 (1.13), 1.943 (1.29), 1.950 (1.31), 1.962 (1.48), 1.978 (1.06), 1.984 (0.99), 1.997 (0.84), 2.010 (0.83), 2.031 (1.10), 2.043 (1.59), 2.059 (1.72), 2.079 (1.25), 2.130 (1.19), 2.144 (1.58), 2.150 (1.65), 2.164 (1.81), 2.176 (1.03), 2.182 (1.06), 2.197 (0.82), 2.546 (0.57), 2.561 (0.78), 2.565 (0.83), 2.578 (2.20), 2.593 (2.38), 2.597 (2.69), 2.640 (2.79), 2.647 (3.55), 2.670 (3.98), 2.734 (0.88), 2.755 (1.78), 2.772 (1.80), 2.793 (0.97), 2.874 (1.02), 2.895 (2.15), 2.905 (4.76), 2.929 (3.71), 3.519 (1.80), 3.530 (2.04), 3.541 (1.94), 3.552 (1.72), 4.132 (0.55), 4.147 (0.78), 4.151 (0.80), 4.159 (2.39), 4.166 (1.09), 4.174 (4.83), 4.178 (3.62), 4.194 (4.76), 4.202 (1.11), 4.210 (2.37), 4.218 (0.83), 4.221 (0.84), 4.237 (0.55), 5.008 (7.89), 5.310 (12.43), 6.171 (11.84), 6.923 (7.18), 7.007 (0.41), 7.529 (0.43), 8.107 (5.26), 8.112 (5.33), 8.604 (5.00), 8.607 (4.94), 9.256 (1.04).

### Example 924

### 5-{(3R)-1-[cyclopropyl(1H-1,2,4-triazol-5-yl)methyl]-5′,6′-dihydrospiro[pyrrolidine-3,4′-pyrrolo[1,2-b]pyrazol]-2′-yl}-3-[(1R)-1-(3-fluoropyridin-2-yl)ethoxy]pyridin-2-amine (Diastereomer 1 and Diastereomer 2)

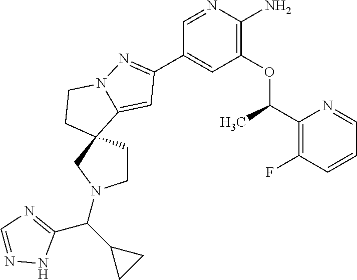 [SMILES: C[C@@H](Oc1cc(-c2cc3n(n2)CC[C@@]32CCN(C(c3ncn[nH]3)C3CC3)C2)cnc1N)c1ncccc1F]

5-[(3R)-5′,6′-Dihydrospiro[pyrrolidine-3,4′-pyrrolo[1,2-b]pyrazol]-2′-yl]-3-[(1R)-1-(3-fluoropyridin-2-yl)ethoxy]pyridin-2-amine hydrogen chloride (1/1) (76.0 mg, 176 μmol) and cyclopropyl(1H-1,2,4-triazol-5-yl)methanone (36.3 mg, 265 μmol) were dissolved in methanol (1.9 mL). N,N-Diisopropylethylamine (120 μL, 710 μmol) and titanium(IV) isopropoxide (160 μL, 530 μmol) were added and stirred for 1.5 h at 60° C. The reaction mixture was allowed to cool down, sodium cyanoborohydride (33.3 mg, 529 μmol) was added and the mixture was stirred for 16 h at 60° C. The reaction mixture was allowed to cool down, diluted with water (0.5 mL) and stirred for 15 min. The suspension was filtered and washed with methanol. The filtrate was concentrated. The residue was purified by preparative HPLC to give 43.1 mg (99% purity, 47% yield) of the title compound.

LC-MS (Method 1): Rₜ=0.80 min; MS (ESIpos): m/z=516 [M+H]⁺

¹H NMR (DMSO-d₆, 400 MHz): δ (ppm) 13.80 (br s, 1H), 8.44 (dt, 1H), 7.89 (d, 1H), 7.74 (ddd, 1H), 7.42-7.49 (m, 1H), 7.32 (t, 1H), 6.20 (s, 1H), 5.74-5.81 (m, 1H), 5.66 (s, 2H), 3.98-4.10 (m, 2H), 2.92-3.00 (m, 1H), 2.86 (t, 1H), 2.73-2.81 (m, 1H), 2.53-2.69 (m, 3H), 2.40-2.47 (m, 1H), 1.88-2.04 (m, 2H), 1.69 (d, 3H), 1.21-1.32 (m, 1H), 0.60-0.69 (m, 1H), 0.31-0.45 (m, 2H), 0.04-0.13 (m, 1H).

### Example 925

### 5-{(3R)-1-[(3,3-difluorocyclobutyl)(4H-1,2,4-triazol-3-yl)methyl]-5′,6′-dihydrospiro[pyrrolidine-3,4′-pyrrolo[1,2-b]pyrazol]-2′-yl}-3-(trifluoromethyl)pyridin-2-amine (Diastereomer 1)

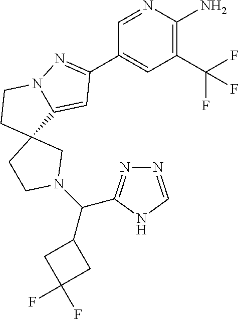 [SMILES: Nc1ncc(-c2cc3n(n2)CC[C@@]32CCN(C(c3nnc[nH]3)C3CC(F)(F)C3)C2)cc1C(F)(F)F]

The compound 5-{(3R)-1-[(3,3-difluorocyclobutyl)(4H-1,2,4-triazol-3-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2, Example 910) was separated into diastereomers by preparative chiral HPLC to give 20.0 mg (6% yield) of the title compound (diastereomer 1: Rₜ=5.2-6.5 min).

Preparative Chiral HPLC Method:

Instrument: Sepiatec: Prep SFC100; Column: Chiralpak IG 5μ, 250×50; eluent A: CO2; eluent B: methanol+0.2 vol % aqueous ammonia (32%); isocratic: 20% B; flow: 100 mL/min; temperature: 40° C.; BPR: 150 bar; UV: 271 nm

Analytical Chiral HPLC Method:

Instrument: Waters acquity UPC2 QDA; Column: Chiralpak IG 3μ, 100×4.6; eluent A: CO2; eluent B: methanol+0.2 vol % aqueous ammonia (32%); isocratic: 20% B; flow: 4 mL/min; temperature: 40° C.; BPR: 1800 psi; UV: 280 nm

Analytical chiral HPLC Rₜ=1.83 min

¹H-NMR (400 MHz, DMSO-d6) δ [ppm]: 0.898 (0.71), 0.916 (1.55), 0.935 (0.78), 1.232 (0.78), 1.276 (2.46), 1.294 (2.46), 1.363 (6.28), 1.885 (1.49), 1.904 (1.62), 1.921 (1.10), 2.074 (2.46), 2.105 (0.71), 2.144 (0.78), 2.318 (1.23), 2.323 (2.79), 2.327 (3.95), 2.331 (2.85), 2.336 (1.23), 2.356 (0.45), 2.374 (0.97), 2.390 (1.36), 2.406 (2.07), 2.425 (1.55), 2.450 (2.20), 2.518 (16.00), 2.523 (11.34), 2.660 (2.33), 2.665 (3.95), 2.669 (5.31), 2.673 (4.34), 2.678 (2.85), 2.724 (2.14), 2.745 (2.14), 2.777 (4.73), 2.799 (3.43), 3.857 (0.97), 4.066 (3.43), 4.084 (7.00), 4.101 (3.30), 5.582 (0.45), 5.600 (0.39), 6.302 (13.41), 6.513 (8.55), 7.264 (0.65), 7.284 (0.52), 7.360 (0.45), 7.507 (0.45), 7.988 (5.25), 7.993 (5.31), 8.564 (4.92), 8.567 (4.86), 13.929 (1.55).

### Example 926

### 5-{(3R)-1-[(3,3-difluorocyclobutyl)(4H-1,2,4-triazol-3-yl)methyl]-5′,6′-dihydrospiro[pyrrolidine-3,4′-pyrrolo[1,2-b]pyrazol]-2′-yl}-3-(trifluoromethyl)pyridin-2-amine (Diastereomer 2)

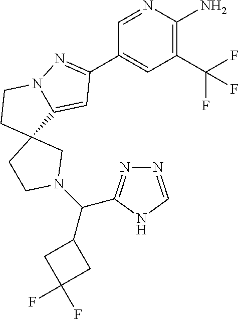 [SMILES: Nc1ncc(-c2cc3n(n2)CC[C@@]32CCN(C(c3nnc[nH]3)C3CC(F)(F)C3)C2)cc1C(F)(F)F]

The diastereomers were separated by preparative chiral HPLC (method see Example 925) to give 17.0 mg (6% yield) of the title compound (diastereomer 2 Rₜ=9.0-10.6 min).

Analytical chiral HPLC (method see Example 925): Rₜ=3.19 min

LC-MS (Method 1): Rₜ=0.89 min; MS (ESIpos): m/z=495 [M+H]⁺

¹H-NMR (400 MHz, DMSO-d6) δ [ppm]: 1.233 (0.67), 1.877 (1.00), 1.894 (2.07), 1.912 (1.13), 2.336 (1.20), 2.385 (0.47), 2.400 (0.73), 2.418 (1.47), 2.435 (1.27), 2.449 (1.93), 2.518 (16.00), 2.523 (11.47), 2.624 (1.33), 2.646 (1.73), 2.678 (1.53), 2.687 (0.73), 2.709 (0.80), 2.728 (1.67), 2.750 (1.27), 2.791 (0.80), 2.838 (0.53), 2.855 (0.87), 2.877 (0.73), 3.863 (0.47), 4.061 (1.60), 4.079 (3.33), 4.096 (1.53), 6.251 (6.47), 6.514 (4.00), 7.983 (2.33), 7.988 (2.40), 8.556 (2.20), 8.560 (2.20).

### Example 927

### 5-{(3R)-1-[cyclopentyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (Diastereomer 1 and Diastereomer 2)

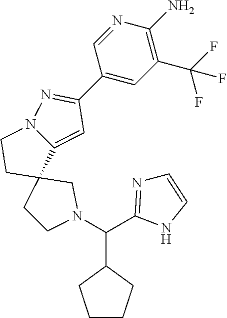 [SMILES: Nc1ncc(-c2cc3n(n2)CC[C@@]32CCN(C(c3ncc[nH]3)C3CCCC3)C2)cc1C(F)(F)F]

5-[(3R)-5′,6′-Dihydrospiro[pyrrolidine-3,4′-pyrrolo[1,2-b]pyrazol]-2′-yl]-3-(trifluoromethyl)pyridin-2-amine hydrogen chloride (1/1) (80.0 mg, 222 μmol) and cyclopentyl(1H-imidazol-2-yl)methanone (54.8 mg, 334 μmol, Intermediate 571) were provided in methanol (1.9 mL). N,N-Diisopropylethylamine (160 μL, 890 μmol) and titanium(IV) isopropoxide (200 μL, 670 μmol) were added and stirred for 2 h at 60° C. Sodium cyanoborohydride (41.9 mg, 667 μmol) was added and the mixture was stirred for 3 days at 60° C. 5-[(3R)-5′,6′-dihydrospiro[pyrrolidine-3,4′-pyrrolo[1,2-b]pyrazol]-2′-yl]-3-(trifluoromethyl)pyridin-2-amine hydrogen chloride (1/1) (40.0 mg, 111 μmol) was added and the reaction mixture was stirred for 7 days at 60° C. The reaction mixture was allowed to cool down, diluted with ethyl acetate and washed with a saturated aqueous sodium bicarbonate solution and brine. The organic phase was dried over a hydrophobic filter and concentrated. The residue was purified by column chromatography (silica gel, dichloromethane/ethanol gradient) to give 53.5 mg (46% yield) of the title compound.

LC-MS (Method): Rₜ=1.08 min; MS (ESIpos): m/z=472 [M+H]⁺

¹H-NMR (400 MHz, DMSO-d6) δ [ppm]: 0.798 (0.81), 0.803 (0.43), 0.815 (0.85), 0.822 (0.89), 0.840 (0.50), 0.886 (0.43), 0.904 (0.89), 0.922 (0.58), 0.968 (0.46), 1.035 (8.85), 1.053 (16.00), 1.070 (9.20), 1.137 (2.13), 1.232 (1.39), 1.368 (0.46), 1.386 (0.58), 1.404 (0.66), 1.422 (0.85), 1.437 (0.93), 1.455 (1.08), 1.472 (1.12), 1.504 (1.55), 1.832 (0.58), 1.846 (1.12), 1.865 (1.51), 1.877 (0.85), 1.907 (0.43), 2.115 (0.89), 2.323 (0.77), 2.327 (1.08), 2.332 (0.81), 2.336 (0.43), 2.371 (0.43), 2.388 (0.77), 2.394 (0.54), 2.406 (0.50), 2.412 (0.73), 2.431 (0.58), 2.441 (0.73), 2.449 (0.70), 2.458 (0.73), 2.466 (0.85), 2.518 (4.52), 2.523 (3.25), 2.620 (0.46), 2.639 (1.24), 2.660 (1.86), 2.669 (1.62), 2.673 (1.00), 2.679 (0.54), 2.685 (0.46), 2.731 (0.93), 2.753 (0.62), 2.790 (0.97), 2.812 (1.00), 2.831 (0.54), 2.850 (0.50), 3.404 (1.28), 3.417 (1.35), 3.422 (3.75), 3.435 (3.86), 3.440 (3.40), 3.452 (3.48), 3.457 (1.35), 3.469 (1.24), 3.483 (1.66), 3.509 (1.51), 4.057 (1.20), 4.074 (2.43), 4.091 (1.16), 4.347 (2.36), 4.359 (4.56), 4.372 (2.20), 6.148 (3.21), 6.220 (3.63), 6.511 (3.79), 6.844 (0.54), 7.036 (0.50), 7.975 (2.20), 8.549 (2.01), 11.759 (0.62).

### Example 928

### 5-{(3′R)-1′-[cyclopropyl(1H-1,2,3-triazol-5-yl)methyl]-6,7-dihydrospiro[pyrazolo[5,1-c][1,4]oxazine-4,3′-pyrrolidin]-2-yl}-3-(trifluoromethyl)pyridin-2-amine (Diastereomer 1 and Diastereomer 2)

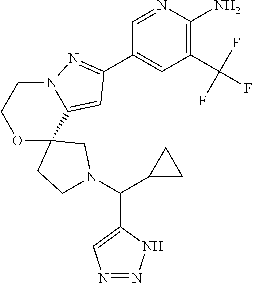 [SMILES: Nc1ncc(-c2cc3n(n2)CCO[C@@]32CCN(C(c3cnn[nH]3)C3CC3)C2)cc1C(F)(F)F]

5-[(3′R)-6,7-Dihydrospiro[pyrazolo[5,1-c][1,4]oxazine-4,3′-pyrrolidin]-2-yl]-3-(trifluoromethyl)pyridin-2-amine hydrogen chloride (1/1) (462 mg, 93% purity, 1.14 mmol) and cyclopropyl(1H-1,2,3-triazol-5-yl)methanone (235 mg, 1.71 mmol) were dissolved in methanol (12 mL). N,N-Diisopropylethylamine (990 μL, 5.7 mmol) and titanium(IV) isopropoxide (1.0 mL, 3.4 mmol) were added and stirred for 2 h at 60° C. The reaction mixture was allowed to cool down, sodium cyanoborohydride (179 mg, 2.86 mmol) was added and stirred over night at 60° C. The reaction mixture was allowed to cool down, diluted with water and stirred for 10 min. The suspension was filtered and the residue was washed with methanol. The filtrate was concentrated. The aqueous residue was extracted with ethyl acetate. The combined organic layers were washed with brine, dried and concentrated. The crude product was purified by preparative HPLC to give 129 mg (95% purity, 23% yield) of the title compound.

LC-MS (Method 1): Rₜ=0.76 min; MS (ESIpos): m/z=461 [M+H]⁺

¹H-NMR (400 MHz, DMSO-d6) δ [ppm]: 0.011 (2.23), 0.311 (2.28), 0.373 (2.23), 0.624 (2.08), 0.816 (0.63), 1.174 (2.33), 1.196 (3.54), 2.039 (1.26), 2.049 (1.60), 2.064 (2.23), 2.081 (2.91), 2.099 (1.75), 2.137 (2.42), 2.153 (1.89), 2.288 (2.23), 2.292 (3.01), 2.297 (2.23), 2.483 (12.90), 2.488 (8.34), 2.505 (0.87), 2.630 (4.90), 2.634 (5.43), 2.639 (3.88), 2.650 (2.38), 2.667 (0.92), 2.714 (2.62), 2.740 (2.81), 2.774 (3.10), 2.796 (5.33), 2.818 (2.86), 2.872 (5.62), 2.902 (1.55), 2.924 (1.21), 3.087 (2.86), 3.112 (2.47), 3.598 (0.48), 3.913 (0.68), 3.931 (1.31), 3.947 (2.86), 3.960 (4.17), 3.972 (4.02), 3.982 (5.19), 3.993 (6.21), 4.015 (4.70), 4.025 (8.48), 4.033 (6.79), 6.537 (16.00), 6.592 (8.73), 7.696 (0.63), 8.001 (9.55), 8.006 (9.79), 8.580 (9.41), 8.584 (9.41).

### Example 929

5-{(3R)-1-[cyclopropyl(1-methyl-1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (Diastereomer 2)

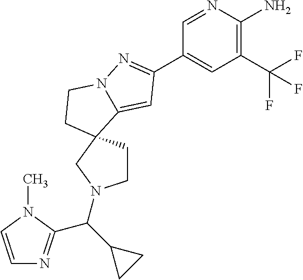 [SMILES: Cn1ccnc1C(C1CC1)N1CC[C@@]2(CCn3nc(-c4cnc(N)c(C(F)(F)F)c4)cc32)C1]

5-{(3R)-1-[Cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (stereoisomer 2) (50.0 mg, 113 μmol, Example 591) was dissolved in DMF (910 μL) under argon. Potassium carbonate (18.7 mg, 135 μmol) and methyliodide (8.4 μL, 140 μmol) were added and the reaction mixture was stirred for 4 h at rt. Potassium carbonate (7.79 mg, 56.4 μmol) and methyliodide (3.5 μL, 56 μmol) were added and the reaction mixture was stirred for 1 h at rt. Water was added and the aqueous phase was extracted three times with dichloromethane. The combined organic layers were washed with brine and dried through a hydrophobic filter. It was concentrated and purified by HPLC to yield 6.50 mg (98% purity, 12% yield) of the title compound.

[α]²⁰_{D}: +32.6° (c=1.00, methanol)

LC-MS (Method 1): Rₜ=1.04 min; MS (ESIpos): m/z=458 [M+H]⁺

¹H NMR (400 MHz, DMSO-d₆, 22° C.) δ ppm 0.04 (dq, 1H), 0.29-0.45 (m, 2H), 0.62-0.71 (m, 1H), 1.25-1.36 (m, 1H), 1.95-2.08 (m, 2H), 2.54-2.60 (m, 2H), 2.81 (dd, 2H), 2.88-2.96 (m, 1H), 3.80 (s, 3H), 4.03-4.14 (m, 2H), 6.41 (s, 1H), 6.52 (s, 2H), 6.71 (d, 1H), 7.03 (d, 1H), 8.00 (d, 1H), 8.58 (d, 1H).

In analogy to the procedures described above, the following examples were prepared using the appropriate intermediates as starting materials.

TABLE 2
Example 930-973

| | Structure | |
|---|---|---|
| | IUPAC-Name | Yield |
| Example | NMR | Analytics |
| Example 930 | 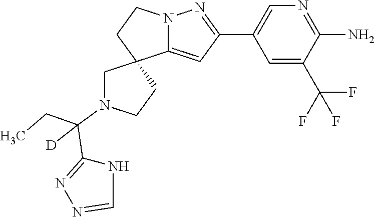 [SMILES: [2H]C(CC)(c1nnc[nH]1)N1CC[C@@]2(CCn3nc(-c4cnc(N)c(C(F)(F)F)c4)cc32)C1] | (90% purity, 54% yield) LC-MS (Method 1): Rₜ = 0.80 min; MS (ESlpos): m/z = 434 [M + H]⁺ |
| | 5-{(3R)-1-[1-(4H-1,2,4-triazol-3-yl)(1-²H)propyl]-5′,6′- | |
| | dihydrospiro[pyrrolidine-3,4′-pyrrolo[1,2-b]pyrazol]-2′-yl}-3- | |
| | (trifluoromethyl)pyridin-2-amine (diastereomer 1 and diastereomer 2) | |
| | ¹H-NMR (400 MHz, DMSO-d6) δ [ppm]: 0.758 (3.19), 0.775 (6.69), | |
| | 0.793 (3.84), 0.814 (1.20), 0.821 (1.19), 0.840 (0.56), 0.886 (0.48), | |
| | 0.904 (0.99), 0.922 (0.55), 1.035 (8.42), 1.052 (16.00), 1.070 (8.79), | |
| | 1.089 (0.45), 1.159 (0.56), 1.196 (1.60), 1.212 (1.69), 1.241 (2.27), | |
| | 1.258 (3.09), 1.274 (1.56), 1.871 (1.57), 1.907 (2.64), 1.928 (2.50), | |
| | 2.323 (1.22), 2.327 (1.74), 2.331 (1.25), 2.412 (0.70), 2.450 (1.08), | |
| | 2.518 (10.69), 2.523 (7.52), 2.665 (2.81), 2.669 (3.09), 2.673 (2.30), | |
| | 2.799 (0.87), 3.404 (1.00), 3.417 (1.12), 3.422 (2.72), 3.435 (2.81), | |
| | 3.439 (2.70), 3.452 (2.76), 3.457 (1.01), 3.469 (0.94), 4.065 (1.62), | |
| | 4.073 (2.02), 4.082 (3.40), 4.090 (3.71), 4.099 (2.06), 4.108 (1.79), | |
| | 4.345 (1.51), 4.358 (2.97), 4.370 (1.46), 6.317 (1.19), 6.343 (1.41), | |
| | 6.519 (7.87), 7.890 (1.08), 7.992 (4.78), 8.514 (0.47), 8.565 (4.20), | |
| | 13.830 (1.04). | |
| Example 931 | 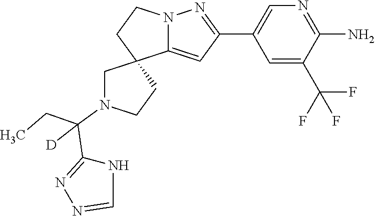 [SMILES: [2H]C(CC)(c1nnc[nH]1)N1CC[C@@]2(CCn3nc(-c4cnc(N)c(C(F)(F)F)c4)cc32)C1] | (98% purity, 9% yield) Rₜ = 4.68 min; Instrument: Thermo Fisher UltiMate 3000; Column: YMC Amylose SA 3μ, 100 × 4.6; eluent A: hexane + 0.1 vol % |
| | 5-{(3R)-1-[1-(4H-1,2,4-triazol-3-yl)(1-²H)propyl]-5′,6′- | diethylamine; |
| | dihydrospiro[pyrrolidine-3,4′-pyrrolo[1,2-b]pyrazol]-2′-yl}-3- | eluent B: |
| | (trifluoromethyl)pyridin-2-amine (diastereomer 2) | ethanol; |
| | Instrument: PrepCon Labomatic HPLC-3; Column: YMC Amylose | isocratic: |
| | SA 5μ, 250 × 30; eluent A: hexane + 0.1 vol %; diethylamine; eluent | 80% A + 20% B; |
| | B: ethanol ; isocratic: 80% A + 20% B; flow: 80 mL/min; temperature: | flow: 1.4 |
| | 25° C.; UV: 280 nm; | mL/min; |
| | ¹H-NMR (400 MHz, DMSO-d6) δ [ppm]: 0.762 (6.65), 0.780 (16.00), | temperature: |
| | 0.798 (7.06), 1.021 (0.54), 1.039 (1.03), 1.057 (0.57), 1.801 (0.80), | 25° C.; UV: |
| | 1.819 (1.25), 1.834 (2.65), 1.853 (2.66), 1.859 (3.08), 1.877 (2.98), | 280 nm; |
| | 1.896 (3.89), 1.913 (6.34), 1.931 (3.37), 2.075 (1.13), 2.322 (1.31), | |
| | 2.327 (1.90), 2.332 (1.37), 2.394 (0.65), 2.411 (1.40), 2.425 (1.79), | |
| | 2.444 (2.89), 2.462 (2.96), 2.518 (7.28), 2.523 (5.16), 2.614 (1.01), | |
| | 2.632 (2.70), 2.654 (2.98), 2.672 (14.54), 2.850 (1.06), 2.866 (2.06), | |
| | 2.889 (2.13), 2.906 (0.86), 4.061 (3.55), 4.078 (7.24), 4.097 (3.61), | |
| | 6.301 (14.83), 6.513 (10.41), 6.564 (0.58), 7.988 (5.93), 7.993 | |
| | (6.09), 8.133 (0.70), 8.561 (5.57), 8.565 (5.46), 13.763 (0.21), | |
| | 13.771 (0.22), 13.776 (0.22), 13.779 (0.23), 13.787 (0.24), 13.790 | |
| | (0.24), 13.797 (0.24), 13.804 (0.25), 13.816 (0.24), 13.822 (0.24), | |
| | 13.832 (0.22). | |
| Example 932 | 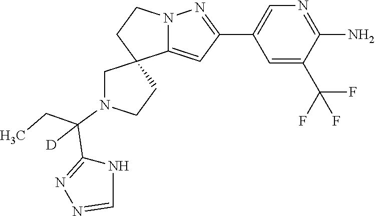 [SMILES: [2H]C(CC)(c1nnc[nH]1)N1CC[C@@]2(CCn3nc(-c4cnc(N)c(C(F)(F)F)c4)cc32)C1] | (100% purity, 16 % yield) Rₜ = 0.83 min; Instrument: Thermo Fisher UltiMate 3000; Column: YMC Amylose SA 3μ, 100 × 4.6; eluent A: hexane + 0.1 vol % |
| | 5-{(3R)-1-[1-(4H-1,2,4-triazol-3-yl)(1-²H)propyl]-5′,6′- | diethylamine; |
| | dihydrospiro[pyrrolidine-3,4′-pyrrolo[1,2-b]pyrazol]-2′-yl}-3- | eluent B: |
| | (trifluoromethyl)pyridin-2-amine (diastereomer 1) | ethanol; |
| | Instrument: PrepCon Labomatic HPLC-3; Column: YMC Amylose | isocratic: |
| | SA 5μ, 250 × 30; eluent A: hexane + 0.1 vol %; diethylamine; eluent | 80% A + 20% B; |
| | B: ethanol ; isocratic: 80% A + 20% B; flow: 80 mL/min; temperature: | flow: 1.4 |
| | 25° C.; UV: 280 nm; | mL/min; |
| | ¹H-NMR (400 MHz, DMSO-d6) δ [ppm]: 0.758 (6.95), 0.777 (16.00), | temperature: |
| | 0.795 (7.29), 1.231 (0.42), 1.801 (0.64), 1.819 (1.25), 1.835 (3.13), | 25° C.; UV: |
| | 1.853 (3.99), 1.870 (3.19), 1.896 (3.17), 1.914 (3.07), 1.932 (1.61), | 280 nm; |
| | 2.318 (0.74), 2.322 (1.61), 2.327 (2.36), 2.332 (1.68), 2.336 (0.73), | |
| | 2.383 (0.53), 2.401 (1.30), 2.416 (1.59), 2.433 (2.55), 2.450 (1.46), | |
| | 2.518 (9.93), 2.523 (7.24), 2.606 (2.40), 2.628 (2.84), 2.644 (0.97), | |
| | 2.665 (3.45), 2.669 (3.82), 2.673 (2.60), 2.679 (2.03), 2.683 (2.03), | |
| | 2.703 (0.83), 2.768 (1.07), 2.789 (2.21), 2.809 (5.27), 2.832 (3.53), | |
| | 4.070 (4.69), 4.087 (9.79), 4.104 (4.56), 6.333 (11.23), 6.513 | |
| | (11.34), 6.547 (0.63), 7.992 (6.88), 7.997 (6.90), 8.566 (6.22), 8.570 | |
| | (6.04), 13.807 (1.76). | |
| Example 933 | 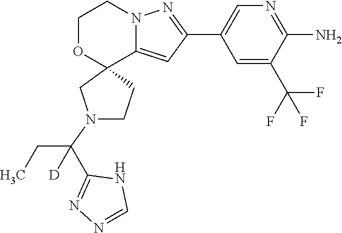 [SMILES: [2H]C(CC)(c1nnc[nH]1)N1CC[C@]2(C1)OCCn1nc(-c3cnc(N)c(C(F)(F)F)c3)cc12] | (98% purity, 2% yield) LC-MS (Method 1): Rₜ = 0.82 min; MS (ESlpos): m/z = 450 [M + H]⁺ |
| | 5-{(3′R)-1′-[1-(4H-1,2,4-triazol-3-yl)(1-²H)propyl]-6,7- | |
| | dihydrospiro[pyrazolo[5,1-c][1,4]oxazine-4,3′-pyrrolidin]-2-yl}-3- | |
| | (trifluoromethyl)pyridin-2-amine (diastereomer 1 and diastereomer 2) | |
| | ¹H-NMR (400 MHz, DMSO-d6) δ [ppm]: 0.729 (4.93), 0.733 (4.28), | |
| | 0.747 (11.56), 0.751 (9.12), 0.765 (5.51), 1.232 (0.77), 1.752 (0.60), | |
| | 1.798 (0.49), 1.814 (0.93), 1.831 (2.17), 1.849 (2.85), 1.860 (1.88), | |
| | 1.866 (2.06), 1.878 (1.56), 1.905 (0.61), 2.017 (0.57), 2.030 (1.03), | |
| | 2.049 (1.34), 2.062 (1.71), 2.074 (0.98), 2.081 (1.36), 2.095 (1.46), | |
| | 2.110 (1.52), 2.125 (1.06), 2.142 (0.59), 2.318 (0.83), 2.323 (1.87), | |
| | 2.327 (2.61), 2.332 (1.80), 2.337 (0.83), 2.518 (10.04), 2.523 (6.88), | |
| | 2.621 (0.56), 2.640 (1.71), 2.660 (2.99), 2.665 (3.08), 2.669 (3.38), | |
| | 2.674 (2.83), 2.679 (1.81), 2.692 (0.49), 2.781 (2.13), 2.807 (3.09), | |
| | 2.820 (2.11), 2.830 (1.54), 2.840 (1.43), 2.871 (0.76), 2.897 (1.86), | |
| | 2.914 (2.71), 2.940 (1.08), 3.056 (2.91), 3.082 (2.61), 3.963 (3.24), | |
| | 3.973 (4.73), 3.986 (3.32), 4.044 (4.34), 4.053 (5.44), 6.509 (3.44), | |
| | 6.529 (5.94), 6.568 (10.11), 8.019 (6.37), 8.024 (6.39), 8.204 | |
| | (16.00), 8.592 (5.49), 8.595 (5.25). | |
| Example 934 | 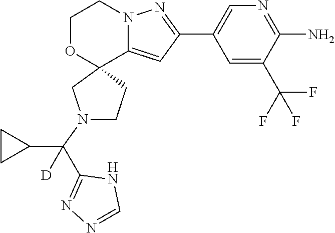 [SMILES: [2H]C(c1nnc[nH]1)(C1CC1)N1CC[C@]2(C1)OCCn1nc(-c3cnc(N)c(C(F)(F)F)c3)cc12] | (90% purity, 19% yield) LC-MS (Method 1): Rₜ = 0.89 min; MS (ESlpos): m/z = 461 [M + H]⁺ |
| | 5-{(3′R)-1′-[cyclopropyl(1H-imidazol-2-yl)(²H)methyl]-6,7- | |
| | dihydrospiro[pyrazolo[5,1-c][1,4]oxazine-4,3′-pyrrolidin]-2-yl}-3- | |
| | (trifluoromethyl)pyridin-2-amine (diatereomer 1 and diastereomer 2) | |
| | ¹H-NMR (400 MHz, DMSO-d6) δ [ppm]: −0.048 (1.37), 0.053 (1.15), | |
| | 0.063 (1.37), 0.076 (1.39), 0.310 (1.09), 0.321 (1.34), 0.331 (1.57), | |
| | 0.335 (1.53), 0.350 (1.82), 0.357 (1.78), 0.370 (1.64), 0.381 (1.21), | |
| | 0.393 (0.65), 0.639 (1.28), 0.798 (0.67), 0.802 (0.41), 0.814 (0.72), | |
| | 0.821 (0.74), 0.886 (0.41), 0.904 (0.79), 1.035 (2.02), 1.052 (4.31), | |
| | 1.070 (2.45), 1.196 (0.40), 1.208 (0.87), 1.229 (2.07), 1.243 (1.71), | |
| | 2.081 (0.78), 2.097 (1.17), 2.114 (1.66), 2.131 (1.04), 2.157 (1.35), | |
| | 2.173 (1.19), 2.180 (0.93), 2.190 (0.70), 2.336 (0.82), 2.518 (16.00), | |
| | 2.523 (11.36), 2.568 (0.81), 2.586 (1.59), 2.607 (1.39), 2.624 (0.59), | |
| | 2.669 (2.70), 2.673 (1.97), 2.678 (1.28), 2.684 (2.21), 2.780 (1.18), | |
| | 2.794 (0.59), 2.807 (2.09), 2.829 (0.88), 2.923 (1.78), 2.949 (1.79), | |
| | 2.974 (0.63), 3.184 (1.89), 3.210 (1.70), 3.422 (0.90), 3.435 (0.92), | |
| | 3.440 (0.88), 3.452 (0.89), 3.959 (0.56), 3.973 (1.12), 3.992 (1.44), | |
| | 4.004 (1.67), 4.020 (2.62), 4.031 (3.03), 4.051 (1.98), 4.063 (4.48), | |
| | 4.072 (2.97), 4.345 (0.57), 4.358 (1.09), 4.370 (0.55), 6.579 (7.90), | |
| | 6.622 (6.55), 6.628 (9.01), 6.794 (0.40), 8.027 (4.38), 8.032 (4.46), | |
| | 8.613 (4.52), 8.618 (4.43), 11.889 (1.35). | |
| Example 935 | 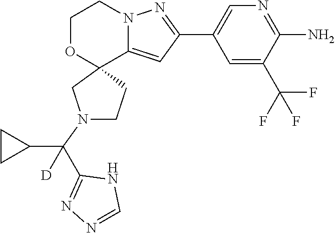 [SMILES: [2H]C(c1nnc[nH]1)(C1CC1)N1CC[C@]2(C1)OCCn1nc(-c3cnc(N)c(C(F)(F)F)c3)cc12] | (98% purity, 18% yield) Rₜ = 1.72 min; Instrument: Thermo Fisher UltiMate 3000; Column: YMC Amylose SA 3μ, 100 × 4.6; eluent A: hexane + 0.1 vol % diethylamine; |
| | 5-{(3'R)-1′-[cyclopropyl(1H-imidazol-2-yl)(²H)methyl]-6,7- | eluent B: 2- |
| | dihydrospiro[pyrazolo[5,1-c][1,4]oxazine-4,3′-pyrrolidin]-2-yl}-3- | propanol; |
| | (trifluoromethyl)pyridin-2-amine (diastereomer 1) | isocratic: |
| | Instrument: PrepCon Labomatic HPLC-3; Column: YMC Amylose | 60% B + 40% B; |
| | SA 5μ, 250 × 30; eluent A: hexane + 0.1 vol %; diethylamine; eluent | flow: 1.4 |
| | B: 2-propanol; isocratic: 60% A + 40% B; flow: 60 mL/min; | mL/min; |
| | temperature: 25° C.; UV: 280 nm; | temperature: |
| | ¹H-NMR (400 MHz, DMSO-d6) δ [ppm]: 0.030 (0.51), 0.041 (1.13), | 25° C.; UV: |
| | 0.053 (1.69), 0.065 (1.85), 0.077 (1.44), 0.089 (0.63), 0.288 (0.42), | 280 nm; |
| | 0.302 (0.80), 0.312 (1.31), 0.323 (1.61), 0.334 (1.88), 0.345 (1.73), | |
| | 0.356 (1.98), 0.369 (2.00), 0.381 (1.81), 0.392 (1.18), 0.406 (0.43), | |
| | 0.614 (0.62), 0.623 (0.95), 0.635 (1.42), 0.645 (1.55), 0.658 (1.28), | |
| | 0.669 (0.80), 0.680 (0.50), 1.210 (0.79), 1.223 (1.69), 1.231 (2.74), | |
| | 1.243 (2.63), 1.255 (1.49), 1.263 (1.35), 1.276 (0.61), 2.066 (0.54), | |
| | 2.085 (0.96), 2.100 (1.77), 2.115 (1.95), 2.133 (1.26), 2.145 (1.09), | |
| | 2.163 (2.11), 2.180 (1.84), 2.196 (1.02), 2.213 (0.55), 2.518 (9.00), | |
| | 2.523 (5.99), 2.562 (1.00), 2.582 (1.81), 2.601 (1.88), 2.620 (0.84), | |
| | 2.779 (3.21), 2.805 (4.14), 2.922 (4.88), 2.933 (1.29), 2.949 (5.16), | |
| | 2.972 (1.70), 2.989 (0.77), 3.941 (0.82), 3.958 (1.42), 3.973 (2.90), | |
| | 3.979 (2.23), 3.991 (3.49), 4.004 (2.32), 4.025 (1.14), 4.028 (1.06), | |
| | 4.036 (0.93), 4.050 (4.22), 4.063 (5.43), 6.578 (9.50), 6.622 (16.00), | |
| | 6.765 (0.77), 7.032 (0.79), 8.028 (5.65), 8.033 (5.78), 8.613 (5.34), | |
| | 8.618 (5.29), 11.870 (1.84). | |
| | [α]²⁰_{D}: +53.0° (c = 1.00, methanol | |
| Example 936 | 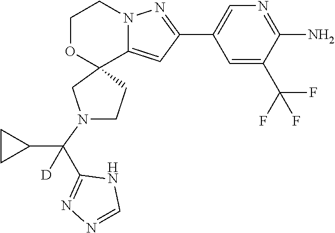 [SMILES: [2H]C(c1nnc[nH]1)(C1CC1)N1CC[C@]2(C1)OCCn1nc(-c3cnc(N)c(C(F)(F)F)c3)cc12] | (99% purity, 20% yield) Rₜ = 3.11 min; Instrument: Thermo Fisher UltiMate 3000; Column: YMC Amylose SA 3μ, 100 × 4.6; eluent A: hexane + 0.1 vol % diethylamine; |
| | 5-{(3'R)-1′-[cyclopropyl(1H-imidazol-2-yl)(²H)methyl]-6,7- | eluent B: 2- |
| | dihydrospiro[pyrazolo[5,1-c][1,4]oxazine-4,3′-pyrrolidin]-2-yl}-3- | propanol; |
| | (trifluoromethyl)pyridin-2-amine (diastereomer 2) | isocratic: |
| | Instrument: PrepCon Labomatic HPLC-3; Column: YMC Amylose | 60% B + 40% B; |
| | SA 5μ, 250 × 30; eluent A: hexane + 0.1 vol %; diethylamine; eluent | flow: 1.4 |
| | B: 2-propanol; isocratic: 60% A + 40% B; flow: 60 mL/min; | mL/min; |
| | temperature: 25° C.; UV: 280 nm; | temperature: |
| | ¹H-NMR (400 MHz, DMSO-d6) δ [ppm]: −0.049 (1.28), 0.038 (0.48), | 25° C.; UV: |
| | 0.049 (1.06), 0.062 (1.65), 0.074 (1.82), 0.084 (1.41), 0.096 (0.61), | 280 nm; |
| | 0.285 (0.41), 0.298 (0.80), 0.309 (1.28), 0.320 (1.60), 0.330 (2.18), | |
| | 0.335 (1.58), 0.349 (2.70), 0.357 (2.08), 0.369 (1.77), 0.380 (1.16), | |
| | 0.393 (0.43), 0.609 (0.59), 0.619 (0.96), 0.631 (1.46), 0.639 (1.51), | |
| | 0.653 (1.28), 0.664 (0.75), 0.674 (0.51), 0.794 (0.65), 0.844 (0.41), | |
| | 0.861 (0.51), 1.006 (0.56), 1.034 (0.57), 1.083 (1.30), 1.196 (0.76), | |
| | 1.208 (1.41), 1.216 (1.60), 1.229 (3.48), 1.248 (1.52), 1.259 (2.36), | |
| | 2.061 (0.56), 2.074 (1.40), 2.081 (1.01), 2.095 (1.65), 2.112 (2.07), | |
| | 2.130 (1.33), 2.140 (1.16), 2.157 (2.06), 2.173 (1.82), 2.189 (0.94), | |
| | 2.206 (0.55), 2.323 (1.07), 2.327 (1.49), 2.331 (1.09), 2.518 (6.17), | |
| | 2.523 (4.08), 2.568 (0.85), 2.586 (1.88), 2.608 (1.96), 2.625 (0.81), | |
| | 2.658 (4.09), 2.665 (1.59), 2.669 (1.79), 2.673 (1.52), 2.684 (4.13), | |
| | 2.793 (0.94), 2.813 (1.75), 2.831 (1.64), 2.850 (0.69), 3.183 (3.78), | |
| | 3.209 (3.37), 4.020 (4.62), 4.031 (5.14), 4.063 (5.08), 4.072 (4.28), | |
| | 4.076 (4.21), 6.578 (9.35), 6.627 (16.00), 6.792 (0.46), 6.927 (0.42), | |
| | 8.026 (5.58), 8.031 (5.60), 8.613 (5.21), 8.617 (5.16), 10.856 (0.66), | |
| | 11.891 (1.30). | |
| Example | 5-{(3'R)-1′-[1-(1H-imidazol-2-yl)(1-²H)propyl]-6,7- | (90% purity, |
| 937 | dihydrospiro[pyrazolo[5,1-c][1,4]oxazine-4,3'-pyrrolidin]-2-yl}-3- | 43% yield) |
| | (trifluoromethyl)pyridin-2-amine (diastereomer 1 and diastereomer 2) | LC-MS: |
| | ¹H-NMR (400 MHz, DMSO-d6) δ [ppm]: 0.692 (3.62), 0.699 (2.85), | (Method 1): |
| | 0.711 (8.35), 0.717 (5.54), 0.729 (4.06), 0.736 (2.52), 0.806 (1.80), | Rₜ = 0.92 min; |
| | 0.825 (3.99), 0.843 (1.90), 1.034 (0.75), 1.052 (1.64), 1.070 (0.82), | MS (ESlpos): |
| | 1.205 (2.72), 1.220 (3.31), 1.236 (5.67), 1.246 (12.80), 1.253 | m/z = 449 |
| | (12.37), 1.262 (16.00), 1.284 (9.41), 1.750 (0.56), 1.769 (0.70), | [M + H]⁺ |
| | 1.784 (0.76), 1.803 (0.99), 1.820 (1.26), 1.838 (1.33), 1.858 (1.17), | |
| | 1.876 (1.62), 1.894 (1.58), 1.909 (1.15), 2.053 (1.04), 2.067 (1.32), | |
| | 2.086 (1.57), 2.145 (1.22), 2.322 (1.82), 2.326 (2.39), 2.331 (1.69), | |
| | 2.518 (14.99), 2.522 (10.25), 2.664 (2.48), 2.668 (2.99), 2.673 | |
| | (2.24), 2.751 (0.89), 2.777 (1.01), 2.849 (1.04), 3.113 (1.89), 3.601 | |
| | (1.55), 3.980 (2.51), 3.992 (3.20), 4.006 (2.28), 4.061 (3.77), 6.507 | |
| | (3.76), 6.540 (7.04), 6.580 (7.25), 7.065 (4.84), 7.084 (3.14), 8.014 | |
| | (4.34), 8.595 (4.35), 8.688 (0.41), 8.692 (0.41), 8.694 (0.40), 8.699 | |
| | (0.37), 12.580 (0.15), 12.589 (0.15), 12.608 (0.14), 12.618 (0.14), | |
| | 12.658 (0.14), 12.672 (0.14), 12.677 (0.14), 12.693 (0.14), 12.696 | |
| | (0.14), 12.703 (0.14). | |
| Example 938 | 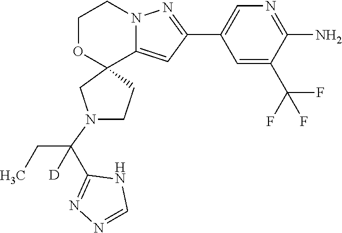 [SMILES: [2H]C(CC)(c1nnc[nH]1)N1CC[C@]2(C1)OCCn1nc(-c3cnc(N)c(C(F)(F)F)c3)cc12] | (97% purity, 16% yield) Rₜ = 1.76 min; Instrument: Thermo Fisher UltiMate 3000; Column: YMC Amylose SA 3μ, 100 × 4.6; eluent A: hexane + 0.1 vol % diethylamine; |
| | 5-{(3'R)-1′-[1-(1H-imidazol-2-yl)(1-²H)propyl]-6,7- | eluent B: 2- |
| | dihydrospiro[pyrazolo[5, 1-c][1,4]oxazine-4,3′-pyrrolidin]-2-yl}-3- | propanol; |
| | (trifluoromethyl)pyridin-2-amine (diastereomer 1) | isocratic: |
| | Instrument: PrepCon Labomatic HPLC-3; Column: YMC Amylose | 70% B + 30% B; |
| | SA 5μ, 250 × 30; eluent A: hexane + 0.1 vol %; diethylamine; eluent | flow: 1.4 |
| | B: 2-propanol; isocratic: 70% A + 30% B; flow: ) mL/min; | mL/min; |
| | temperature: 25° C.; UV: 280 nm; | temperature: |
| | ¹H-NMR (400 MHz, DMSO-d6) δ [ppm]: 0.700 (6.73), 0.719 (16.00), | 25° C.; UV: |
| | 0.736 (7.01), 0.775 (0.96), 0.794 (2.21), 0.813 (1.18), 0.818 (0.98), | 280 nm; |
| | 0.836 (1.03), 0.843 (0.89), 0.861 (1.36), 0.871 (0.45), 0.880 (0.58), | |
| | 1.006 (1.82), 1.026 (2.82), 1.033 (1.63), 1.042 (2.87), 1.083 (4.39), | |
| | 1.099 (0.58), 1.142 (4.57), 1.161 (9.36), 1.179 (4.59), 1.232 (1.69), | |
| | 1.258 (5.55), 1.352 (1.22), 1.391 (0.56), 1.405 (0.46), 1.421 (0.59), | |
| | 1.439 (0.48), 1.443 (0.48), 1.788 (0.57), 1.807 (1.01), 1.826 (1.22), | |
| | 1.841 (2.03), 1.859 (2.16), 1.880 (2.35), 1.899 (2.16), 1.914 (1.29), | |
| | 1.931 (0.90), 2.021 (0.62), 2.039 (1.28), 2.054 (1.43), 2.072 (2.11), | |
| | 2.090 (1.08), 2.130 (0.98), 2.148 (1.63), 2.163 (1.63), 2.180 (1.07), | |
| | 2.195 (0.64), 2.318 (0.76), 2.323 (1.57), 2.327 (2.23), 2.331 (1.61), | |
| | 2.336 (0.75), 2.518 (8.70), 2.523 (5.80), 2.660 (1.75), 2.665 (2.67), | |
| | 2.669 (3.28), 2.673 (2.63), 2.888 (2.60), 2.927 (2.38), 2.994 (1.33), | |
| | 3.942 (0.57), 3.957 (1.59), 3.971 (3.86), 3.982 (4.89), 3.992 (2.93), | |
| | 4.011 (0.69), 4.022 (0.81), 4.049 (4.64), 4.061 (6.77), 4.073 (2.64), | |
| | 6.510 (13.98), 6.584 (10.00), 6.621 (0.46), 7.094 (7.95), 8.012 | |
| | (5.91), 8.017 (6.18), 8.591 (5.53), 8.595 (5.67), 10.854 (0.56). | |
| Example 939 | 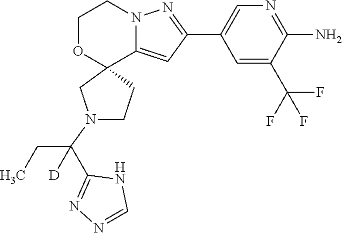 [SMILES: [2H]C(CC)(c1nnc[nH]1)N1CC[C@]2(C1)OCCn1nc(-c3cnc(N)c(C(F)(F)F)c3)cc12] | (99% purity, 23% yield) Rt = 3.54 min; Instrument: Thermo Fisher UltiMate 3000; Column: YMC Amylose SA 3μ, 100 × 4.6; eluent A: hexane + 0.1 vol % diethylamine; |
| | 5-{(3'R)-1′-[1-(1H-imidazol-2-yl)(1-²H)propyl]-6,7- | eluent B: 2- |
| | dihydrospiro[pyrazolo[5,1-c][1,4]oxazine-4,3′-pyrrolidin]-2-yl}-3- | propanol; |
| | (trifluoromethyl)pyridin-2-amine (diastereomer 2) | isocratic: |
| | Instrument: PrepCon Labomatic HPLC-3; Column: YMC Amylose | 70% B + 30% B; |
| | SA 5μ, 250 × 30; eluent A: hexane + 0.1 vol %; diethylamine; eluent | flow: 1.4 |
| | B: 2-propanol; isocratic: 70% A + 30% B; flow: 70 mL/min; | mL/min; |
| | temperature: 25° C.; UV: 280 nm; | temperature: |
| | ¹H-NMR (400 MHz, DMSO-d6) δ [ppm]: 0.686 (6.11), 0.705 (14.76), | 25° C.; UV: |
| | 0.723 (6.54), 0.775 (1.01), 0.794 (2.41), 0.813 (1.19), 0.818 (0.93), | 280 nm; |
| | 0.836 (1.11), 0.843 (0.92), 0.861 (1.46), 0.880 (0.64), 0.984 (0.37), | |
| | 1.006 (2.03), 1.026 (11.91), 1.034 (2.11), 1.042 (11.76), 1.083 | |
| | (4.64), 1.100 (0.59), 1.136 (0.38), 1.154 (0.66), 1.172 (0.39), 1.232 | |
| | (0.98), 1.259 (6.24), 1.352 (0.59), 1.391 (0.60), 1.421 (0.67), 1.440 | |
| | (0.52), 1.527 (0.39), 1.609 (0.36), 1.758 (1.26), 1.776 (1.53), 1.791 | |
| | (2.46), 1.810 (2.15), 1.840 (2.15), 1.859 (2.28), 1.873 (1.48), 1.892 | |
| | (1.08), 2.019 (0.49), 2.038 (1.13), 2.052 (1.43), 2.070 (2.63), 2.074 | |
| | (2.40), 2.089 (2.18), 2.107 (1.85), 2.121 (1.76), 2.137 (0.74), 2.518 | |
| | (7.08), 2.523 (4.64), 2.564 (0.82), 2.582 (2.00), 2.605 (2.08), 2.622 | |
| | (0.77), 2.707 (3.54), 2.733 (3.92), 2.775 (0.89), 2.795 (1.62), 2.810 | |
| | (1.52), 2.829 (0.70), 3.033 (3.61), 3.059 (3.11), 3.764 (0.34), 3.990 | |
| | (4.56), 4.003 (4.21), 4.049 (4.31), 4.062 (4.96), 4.348 (0.81), 4.358 | |
| | (0.82), 6.519 (16.00), 6.574 (9.13), 6.937 (2.74), 8.016 (5.44), 8.021 | |
| | (5.45), 8.598 (5.09), 8.601 (4.99), 10.518 (0.65), 10.638 (0.60), | |
| | 10.854 (1.71), 11.902 (0.28), 11.910 (0.30), 11.915 (0.30), 11.919 | |
| | (0.31), 11.921 (0.31), 11.925 (0.32), 11.929 (0.31), 11.950 (0.29). | |
| Example 940 | 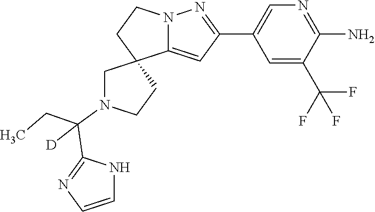 [SMILES: [2H]C(CC)(c1ncc[nH]1)N1CC[C@@]2(CCn3nc(-c4cnc(N)c(C(F)(F)F)c4)cc32)C1] | (90% purity, 58% yield) LC-MS: (Method 1): Rₜ = 0.93 min; MS (ESlpos): m/z = 433 [M + H]⁺ |
| | 5-{(3R)-1-[1-(1H-imidazol-2-yl)(1-²H)propyl]-5′,6′- | |
| | dihydrospiro[pyrrolidine-3,4′-pyrrolo[1,2-b]pyrazol]-2′-yl}-3- | |
| | (trifluoromethyl)pyridin-2-amine (diastereomer 1 and diastereomer 2) | |
| | ¹H-NMR (400 MHz, DMSO-d6) δ [ppm]: 0.716 (3.72), 0.723 (3.87), | |
| | 0.735 (8.79), 0.741 (8.25), 0.753 (4.34), 0.759 (3.80), 0.810 (1.51), | |
| | 0.829 (3.74), 0.847 (1.73), 1.196 (0.74), 1.204 (1.82), 1.213 (0.76), | |
| | 1.220 (2.06), 1.235 (4.16), 1.247 (7.75), 1.253 (11.09), 1.263 | |
| | (11.25), 1.283 (5.93), 1.715 (0.50), 1.733 (0.44), 1.762 (0.52), 1.781 | |
| | (0.54), 1.815 (0.46), 1.832 (0.66), 1.848 (0.95), 1.866 (1.37), 1.884 | |
| | (1.32), 1.907 (2.51), 1.928 (2.20), 1.943 (3.44), 1.960 (4.31), 1.976 | |
| | (2.28), 2.323 (2.01), 2.327 (2.73), 2.331 (1.96), 2.416 (0.51), 2.428 | |
| | (1.09), 2.448 (1.37), 2.461 (2.03), 2.518 (16.00), 2.523 (12.56), | |
| | 2.660 (1.32), 2.665 (2.48), 2.669 (3.32), 2.673 (2.56), 2.678 (1.57), | |
| | 2.716 (1.28), 2.959 (0.87), 3.114 (1.74), 3.133 (1.83), 3.164 (8.77), | |
| | 3.585 (1.33), 3.602 (1.55), 3.618 (1.26), 4.072 (1.56), 4.082 (3.66), | |
| | 4.098 (5.20), 4.115 (2.52), 6.321 (3.67), 6.359 (4.35), 6.532 (9.52), | |
| | 7.149 (5.91), 7.181 (2.26), 7.986 (5.30), 8.135 (0.70), 8.567 (5.23). | |
| Example 941 | 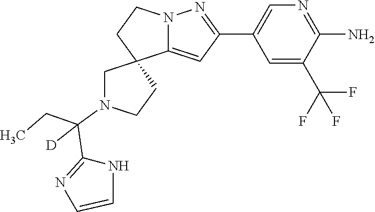 [SMILES: [2H]C(CC)(c1ncc[nH]1)N1CC[C@@]2(CCn3nc(-c4cnc(N)c(C(F)(F)F)c4)cc32)C1] | (100% purity, 30% yield) Rₜ = 3.29 min; Instrument: Waters Acquity UPC2 QDA; Column: Chiral Art Amylose-SA 3μ 100 × 4.6 mm; eluent A: |
| | 5-{(3R)-1-[1-(1H-imidazol-2-yl)(1-²H)propyl]-5′,6′- | CO2; eluent |
| | dihydrospiro[pyrrolidine-3,4′-pyrrolo[1,2-b]pyrazol]-2′-yl}-3- | B: methanol + |
| | (trifluoromethyl)pyridin-2-amine (diastereomer 1) | 0.2 vol % |
| | Instrument: Sepiatec: Prep SFC100; Column: Chiral Art Amylose-SA | aqueous |
| | 5μ 250 × 30 mm; eluent A: CO2; eluent B: methanol + 0.2 vol % | ammonia |
| | aqueous ammonia (32%); isocratic: 10% B; gradient: no; flow: 100 | (32%); |
| | mL/min; temperature: 40° C.; BPR: 150 bar; UV: 267 nm. | isocratic: |
| | ¹H-NMR (400 MHz, DMSO-d6) δ [ppm]: 0.715 (5.89), 0.733 (14.25), | 10% B; |
| | 0.752 (6.31), 0.817 (0.63), 0.868 (0.41), 1.765 (0.88), 1.783 (1.22), | gradient: no; |
| | 1.798 (2.31), 1.816 (2.21), 1.829 (2.39), 1.848 (2.44), 1.862 (1.40), | flow: 4 |
| | 1.866 (1.40), 1.882 (2.06), 1.902 (3.02), 1.918 (2.82), 1.938 (1.55), | mL/min; |
| | 1.949 (0.56), 1.953 (0.60), 1.969 (0.43), 2.385 (0.59), 2.402 (1.38), | temperature: |
| | 2.417 (1.55), 2.435 (2.30), 2.452 (1.48), 2.472 (2.26), 2.518 (6.47), | 40.0° C.; BPR: |
| | 2.523 (4.71), 2.569 (4.24), 2.597 (1.59), 2.615 (1.82), 2.634 (0.83), | 1800 psi; UV: |
| | 2.747 (0.93), 2.768 (1.96), 2.784 (1.73), 2.796 (4.34), 2.819 (3.46), | 280 nm |
| | 3.298 (0.76), 4.064 (3.20), 4.081 (6.16), 4.099 (3.15), 6.328 (16.00), | |
| | 6.335 (0.88), 6.514 (8.98), 6.814 (6.81), 7.034 (3.88), 7.038 (5.19), | |
| | 7.041 (4.03), 7.989 (4.99), 7.994 (5.08), 8.568 (4.67), 8.573 (4.55), | |
| | 11.776 (2.48). | |
| | [α]²⁰_{D}: +69.3° (c = 1.00, methanol) | |
| Example 942 | 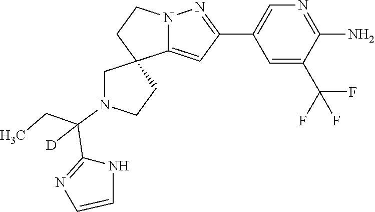 [SMILES: [2H]C(CC)(c1ncc[nH]1)N1CC[C@@]2(CCn3nc(-c4cnc(N)c(C(F)(F)F)c4)cc32)C1] | (99% purity, 30% yield) Rₜ = 4.90 min; Instrument: Waters Acquity UPC2 QDA; Column: Chiral Art Amylose-SA 3μ 100 × 4.6 mm; eluent A: |
| | 5-{(3R)-1-[1-(1H-imidazol-2-yl)(1-²H)propyl]-5',6'- | CO2; eluent |
| | dihydrospiro[pyrrolidine-3,4'-pyrrolo[1,2-b]pyrazol]-2'-yl}-3- | B: methanol + |
| | (trifluoromethyl)pyridin-2-amine (diastereomer 2) | 0.2 vol % |
| | Instrument: Sepiatec: Prep SFC100; Column: Chiral Art Amylose-SA | aqueous |
| | 5μ 250 × 30 mm; eluent A: CO2; eluent B: methanol + 0.2 vol % | ammonia |
| | aqueous ammonia (32%); isocratic: 10% B; gradient: no; flow: 100 | (32%); |
| | mL/min; temperature: 40° C.; BPR: 150 bar; UV: 267 nm. | isocratic: |
| | ¹H-NMR (400 MHz, DMSO-d6) δ [ppm]: 0.722 (5.35), 0.740 (13.18), | 10% B; |
| | 0.758 (5.71), 1.759 (0.86), 1.777 (1.13), 1.793 (2.06), 1.811 (2.05), | gradient: no; |
| | 1.832 (2.15), 1.851 (2.08), 1.866 (1.22), 1.884 (1.05), 1.903 (2.24), | flow: 4 |
| | 1.918 (4.54), 1.937 (2.62), 2.318 (0.41), 2.322 (0.92), 2.326 (1.29), | mL/min; |
| | 2.332 (0.91), 2.394 (0.46), 2.414 (0.85), 2.427 (1.41), 2.446 (1.90), | temperature: |
| | 2.463 (2.38), 2.518 (5.54), 2.522 (3.54), 2.566 (0.79), 2.587 (4.00), | 40.0° C.; BPR: |
| | 2.610 (5.00), 2.625 (0.85), 2.655 (4.65), 2.664 (1.30), 2.669 (1.53), | 1800 psi; UV: |
| | 2.673 (1.52), 2.678 (3.02), 2.832 (0.80), 2.848 (1.46), 2.865 (1.12), | 280 nm |
| | 2.871 (1.50), 2.888 (0.66), 4.039 (0.54), 4.047 (1.71), 4.058 (1.99), | |
| | 4.062 (2.58), 4.066 (2.67), 4.073 (2.43), 4.078 (2.40), 4.081 (2.03), | |
| | 4.092 (1.71), 4.100 (0.55), 4.104 (0.52), 6.284 (16.00), 6.292 (0.76), | |
| | 6.515 (8.08), 6.811 (1.54), 7.045 (1.51), 7.984 (4.58), 7.989 (4.66), | |
| | 8.562 (4.20), 8.567 (4.16), 11.780 (1.32). | |
| | [α]²⁰_{D}: +52.1° (c = 1.00, methanol) | |
| Example 943 | 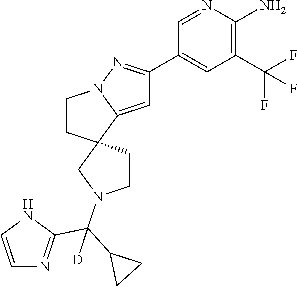 [SMILES: [2H]C(c1ncc[nH]1)(C1CC1)N1CC[C@@]2(CCn3nc(-c4cnc(N)c(C(F)(F)F)c4)cc32)C1] | (98% purity, 49% yield) LC-MS Method 1): Rₜ = 0.92 min; MS (ESlpos): m/z = 445 [M + H]⁺ |
| | 5-{(3R)-1-[cyclopropyl(1H-imidazol-2-yl)(²H)methyl]-5′,6′- | |
| | dihydrospiro[pyrrolidine-3,4′-pyrrolo[1,2-b]pyrazol]-2′-yl}-3- | |
| | (trifluoromethyl)pyridin-2-amine (diastereomer 1 and diastereomer 2) | |
| | ¹H-NMR (400 MHz, DMSO-d6) δ [ppm]: 0.074 (0.71), 0.086 (1.45), | |
| | 0.097 (2.48), 0.110 (2.64), 0.120 (1.78), 0.310 (0.62), 0.334 (1.95), | |
| | 0.350 (3.36), 0.366 (3.92), 0.378 (2.12), 0.393 (1.58), 0.405 (1.54), | |
| | 0.417 (1.39), 0.429 (0.87), 0.620 (1.22), 0.651 (2.28), 0.666 (1.41), | |
| | 1.168 (0.44), 1.187 (1.17), 1.194 (3.71), 1.204 (1.32), 1.210 (4.21), | |
| | 1.240 (4.86), 1.246 (4.42), 1.253 (4.17), 1.907 (2.41), 1.926 (0.41), | |
| | 1.945 (0.74), 1.958 (1.38), 1.978 (2.82), 1.996 (4.14), 2.002 (3.56), | |
| | 2.016 (2.55), 2.035 (0.86), 2.055 (0.46), 2.318 (1.15), 2.323 (2.48), | |
| | 2.327 (3.49), 2.331 (2.51), 2.337 (1.11), 2.452 (1.48), 2.470 (3.80), | |
| | 2.518 (16.00), 2.523 (10.42), 2.558 (1.87), 2.572 (3.19), 2.593 | |
| | (3.95), 2.610 (3.71), 2.625 (3.84), 2.641 (3.09), 2.659 (2.09), 2.665 | |
| | (3.28), 2.669 (4.10), 2.673 (2.85), 2.784 (1.94), 2.805 (2.27), 2.968 | |
| | (2.70), 2.992 (2.15), 3.166 (7.64), 4.026 (0.46), 4.046 (0.75), 4.053 | |
| | (1.55), 4.072 (3.07), 4.091 (5.61), 4.108 (7.22), 4.125 (3.55), 6.437 | |
| | (13.10), 6.446 (15.46), 6.528 (15.01), 6.973 (7.54), 6.984 (6.93), | |
| | 7.999 (7.85), 8.136 (1.05), 8.583 (7.98), 8.587 (7.99). | |
| Example 944 | 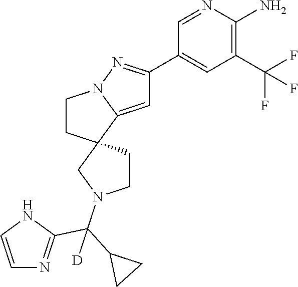 [SMILES: [2H]C(c1ncc[nH]1)(C1CC1)N1CC[C@@]2(CCn3nc(-c4cnc(N)c(C(F)(F)F)c4)cc32)C1] | (100% purity, 22% yield) Rₜ = 1.19 min; Instrument: Waters Acquity UPC2 QDA; Column: Chiralpak IG 3μ 100 × 4.6 mm; eluent A: CO2; eluent B: methanol + 0.2 vol % aqueous ammonia (32%); |
| | 5-{(3R)-1-[cyclopropyl(1H-imidazol-2-yl)(²H)methyl]-5′,6′- | isocratic: |
| | dihydrospiro[pyrrolidine-3,′-pyrrolo[1,2-b]pyrazol]-2′-yl}-3- | 30% B; |
| | (trifluoromethyl)pyridin-2-amine (diastereomer 1) | gradient: no; |
| | Instrument: Sepiatec: Prep SFC100; Column: Chiralpak IG 5μ | flow: 4 |
| | 250 × 30 mm; eluent A: CO2; eluent B: methanol + 0.2 vol % aqueous | mL/min; |
| | ammonia (32%); isocratic: 30% B; gradient: no; flow: 100 mL/min; | temperature: |
| | temperature: 40° C.; BPR: 150 bar; UV: 271 nm. | 40.0° C.; BPR: |
| | ¹H-NMR (400 MHz, DMSO-d6) δ [ppm]: −0.065 (0.40), 0.109 (0.88), | 100 bar; UV: |
| | 0.122 (1.29), 0.134 (1.39), 0.145 (1.08), 0.157 (0.46), 0.353 (1.01), | 280 nm |
| | 0.365 (1.29), 0.376 (1.23), 0.387 (0.79), 0.398 (0.61), 0.411 (0.59), | |
| | 0.422 (1.18), 0.434 (1.45), 0.445 (1.35), 0.457 (0.91), 0.639 (0.47), | |
| | 0.649 (0.77), 0.660 (1.02), 0.672 (1.29), 0.681 (0.94), 1.191 (0.53), | |
| | 1.208 (0.56), 1.218 (0.57), 1.231 (1.33), 1.239 (1.33), 1.251 (1.87), | |
| | 1.263 (1.04), 1.271 (0.94), 1.283 (0.40), 1.906 (0.66), 1.994 (1.89), | |
| | 2.009 (4.40), 2.028 (2.56), 2.318 (0.76), 2.322 (1.80), 2.326 (2.47), | |
| | 2.332 (1.78), 2.336 (0.79), 2.461 (1.07), 2.518 (9.71), 2.522 (6.51), | |
| | 2.561 (1.68), 2.566 (1.58), 2.580 (1.68), 2.593 (0.95), 2.597 (0.79), | |
| | 2.613 (0.64), 2.660 (2.58), 2.664 (3.58), 2.669 (4.28), 2.673 (3.40), | |
| | 2.678 (2.44), 2.684 (2.45), 2.811 (2.11), 2.834 (1.68), 2.973 (0.78), | |
| | 2.989 (1.38), 3.010 (1.34), 3.028 (0.79), 4.030 (0.51), 4.046 (0.71), | |
| | 4.050 (0.76), 4.057 (1.77), 4.073 (3.48), 4.093 (3.26), 4.108 (1.75), | |
| | 4.115 (0.78), 4.120 (0.68), 4.135 (0.47), 6.442 (16.00), 6.525 (8.42), | |
| | 7.060 (6.79), 7.993 (4.84), 7.998 (4.95), 8.133 (0.59), 8.580 (4.45), | |
| | 8.584 (4.45). | |
| | [α]²⁰_{D}: +33.5° (c = 1.00, methanol) | |
| Example 945 | 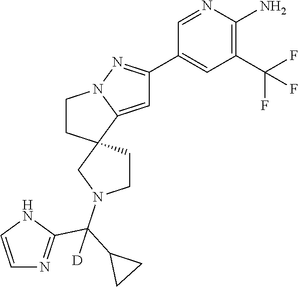 [SMILES: [2H]C(c1ncc[nH]1)(C1CC1)N1CC[C@@]2(CCn3nc(-c4cnc(N)c(C(F)(F)F)c4)cc32)C1] | (99% purity, 22% yield) Rₜ = 2.45 min; Instrument: Waters Acquity UPC2 QDA; Column: Chiralpak IG 3μ 100 × 4.6 mm; eluent A: CO2; eluent B: methanol + 0.2 vol % aqueous ammonia (32%); |
| | 5-{(3R)-1-[cyclopropyl(1H-imidazol-2-yl)(²H)methyl]-5′,6′- | isocratic: |
| | dihydrospiro[pyrrolidine-3,4′-pyrrolo[1,2-b]pyrazol]-2′-yl}-3- | 30% B; |
| | (trifluoromethyl)pyridin-2-amine (diastereomer 2) | gradient: no; |
| | Instrument: Sepiatec: Prep SFC100; Column: Chiralpak IG 5μ | flow: 4 |
| | 250 × 30 mm; eluent A: CO2; eluent B: methanol + 0.2 vol % aqueous | mL/min; |
| | ammonia (32%); isocratic: 30% B; gradient: no; flow: 100 mL/min; | temperature: |
| | temperature: 40° C.; BPR: 150 bar; UV: 271 nm. | 40.0° C.; BPR: |
| | ¹H-NMR (400 MHz, DMSO-d6) δ [ppm]: 0.056 (0.86), 0.069 (1.46), | 100 bar; UV: |
| | 0.083 (1.62), 0.089 (0.84), 0.095 (0.88), 0.301 (0.74), 0.310 (1.34), | 280 nm |
| | 0.321 (2.78), 0.332 (3.01), 0.342 (2.45), 0.354 (1.05), 0.602 (1.03), | |
| | 0.612 (0.97), 0.617 (0.98), 0.624 (1.34), 0.629 (1.19), 0.632 (1.11), | |
| | 0.644 (0.69), 1.202 (0.55), 1.215 (1.05), 1.221 (1.24), 1.227 (0.94), | |
| | 1.235 (1.94), 1.241 (0.91), 1.247 (1.09), 1.254 (0.96), 1.266 (0.44), | |
| | 1.927 (0.63), 1.940 (1.28), 1.956 (1.56), 1.974 (1.87), 1.988 (1.39), | |
| | 1.993 (1.57), 2.008 (1.34), 2.020 (0.63), 2.025 (0.59), 2.040 (0.46), | |
| | 2.442 (0.70), 2.460 (1.73), 2.477 (2.29), 2.518 (7.01), 2.522 (4.27), | |
| | 2.566 (2.05), 2.571 (1.62), 2.583 (2.52), 2.599 (1.87), 2.609 (0.98), | |
| | 2.614 (1.60), 2.632 (0.67), 2.753 (0.82), 2.774 (1.70), 2.790 (1.41), | |
| | 2.796 (0.94), 2.812 (0.58), 2.931 (3.48), 2.954 (3.12), 4.085 (2.89), | |
| | 4.102 (5.49), 4.119 (2.83), 6.440 (16.00), 6.521 (8.09), 6.753 (1.40), | |
| | 7.017 (1.35), 7.999 (4.60), 8.004 (4.60), 8.583 (4.25), 8.587 (4.20), | |
| | 11.784 (1.26). | |
| | [α]²⁰_{D}: +63.3° (c = 1.00, methanol) | |
| Example 946 | 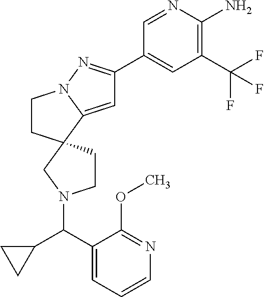 [SMILES: COc1ncccc1C(C1CC1)N1CC[C@@]2(CCn3nc(-c4cnc(N)c(C(F)(F)F)c4)cc32)C1] | (99% purity, 10% yield) LC-MS (Method 1): Rₜ = 1.34 min; MS (ESlpos): m/z = 485 [M + H]⁺ |
| | 5-{(3R)-1-[cyclopropyl(2-methoxypyridin-3-yl)methyl]-5′,6′- | |
| | dihydrospiro[pyrrolidine-3,4′-pyrrolo[1,2-b]pyrazol]-2′-yl}-3- | |
| | (trifluoromethyl)pyridin-2-amine (diastereomer 1 and diastereomer 2) | |
| | ¹H-NMR (400 MHz, DMSO-d6) δ [ppm]: 0.110 (0.89), 0.121 (1.15), | |
| | 0.132 (1.17), 0.146 (0.85), 0.156 (0.52), 0.226 (1.08), 0.236 (1.11), | |
| | 0.336 (0.51), 0.347 (0.63), 0.359 (0.63), 0.379 (0.76), 0.391 (0.89), | |
| | 0.403 (0.80), 0.414 (0.55), 0.621 (1.13), 1.057 (1.07), 1.067 (1.00), | |
| | 1.078 (1.02), 1.964 (0.66), 1.980 (0.74), 2.000 (1.25), 2.013 (2.31), | |
| | 2.033 (1.84), 2.073 (6.05), 2.323 (1.90), 2.327 (2.52), 2.331 (1.92), | |
| | 2.560 (1.56), 2.569 (2.09), 2.576 (2.24), 2.591 (3.02), 2.599 (2.88), | |
| | 2.606 (2.57), 2.629 (1.47), 2.673 (3.85), 2.696 (1.39), 2.738 (0.69), | |
| | 2.753 (0.61), 2.852 (1.26), 2.875 (1.51), 2.898 (2.97), 2.920 (2.48), | |
| | 3.159 (1.24), 3.172 (1.24), 3.826 (16.00), 3.841 (11.69), 4.031 | |
| | (0.46), 4.039 (0.82), 4.059 (1.61), 4.075 (1.74), 4.085 (1.66), 4.101 | |
| | (2.46), 4.112 (0.89), 4.120 (1.21), 6.473 (6.38), 6.478 (4.88), 6.513 | |
| | (5.26), 6.985 (1.00), 6.998 (1.09), 7.004 (1.10), 7.016 (2.28), 7.029 | |
| | (1.51), 7.035 (1.48), 7.048 (1.42), 7.874 (1.09), 7.879 (1.24), 7.889 | |
| | (1.74), 7.893 (2.58), 7.906 (1.49), 7.911 (1.47), 8.017 (4.14), 8.028 | |
| | (1.71), 8.033 (1.50), 8.044 (2.61), 8.048 (2.09), 8.056 (1.85), 8.060 | |
| | (1.61), 8.601 (3.83). | |
| Example 947 | 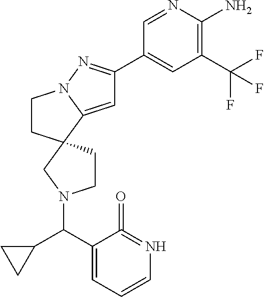 | (100% purity, 50% yield) LC-MS (Method 1): Rₜ = 0.98 min; MS (ESlpos): m/z = 471 [M + H]⁺ |
| | 3-[{(3R)-2′-[6-amino-5-(trifluoromethyl)pyridin-3-yl]-5′,6′-dihydro-1H- | |
| | spiro[pyrrolidine-3,4′-pyrrolo[1,2-b]pyrazol]-1- | |
| | yl}(cyclopropyl)methyl]pyridin-2(1H)-one (diastereomer 1 and | |
| | diastereomer 2) | |
| | ¹H NMR (400 MHz, DMSO-d6, 22° C.) δ ppm 0.17-0.38 (m, 3 H), | |
| | 0.51-0.60 (m, 1 H), 0.91-1.02 (m, 1 H), 1.92-2.08 (m, 2 H), 2.54- | |
| | 2.76 (m, 4 H), 2.83-2.94 (m, 2 H), 4.04-4.14 (m, 2 H), 6.17-6.25 | |
| | (m, 1 H), 6.47 and 6.48 (2s, 1 H), 6.51 and 6.52 (2s, 2 H), 7.20- | |
| | 7.26 (m, 1 H), 7.58 (2t, 1 H), 8.02 (s, 1 H), 8.60 (d, 1 H), 11.49 (br s, | |
| | 1 H). | |
| Example 948 | 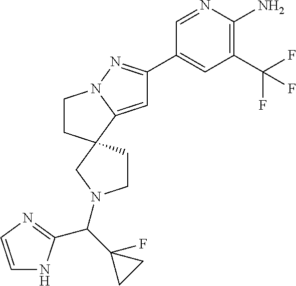 [SMILES: Nc1ncc(-c2cc3n(n2)CC[C@@]32CCN(C(c3ncc[nH]3)C3(F)CC3)C2)cc1C(F)(F)F] | (95% purity, 33% yield) LC-MS (Method 1): Rₜ = 0.94 min; MS (ESlpos): m/z = 462 [M + H]⁺ |
| | 5-{(3R)-1-[(1-fluorocyclopropyl)(1H-imidazol-2-yl)methyl]-5′,6′- | |
| | dihydrospiro[pyrrolidine-3,4′-pyrrolo[1,2-b]pyrazol]-2′-yl}-3- | |
| | (trifluoromethyl)pyridin-2-amine (diastereomer 1 and diastereomer 2) | |
| | ¹H-NMR (400 MHz, DMSO-d6) δ [ppm]: 0.741 (2.51), 0.765 (4.93), | |
| | 0.777 (2.77), 0.788 (3.21), 0.802 (1.62), 0.833 (1.38), 0.865 (1.56), | |
| | 0.883 (1.23), 0.915 (1.42), 1.072 (1.40), 1.098 (1.34), 1.118 (1.52), | |
| | 1.231 (1.78), 2.014 (4.32), 2.030 (4.86), 2.075 (0.75), 2.294 (0.99), | |
| | 2.322 (1.88), 2.327 (2.62), 2.332 (1.93), 2.518 (14.39), 2.522 (9.81), | |
| | 2.583 (1.38), 2.599 (2.64), 2.616 (2.88), 2.631 (2.60), 2.642 (1.31), | |
| | 2.648 (1.45), 2.664 (2.58), 2.669 (3.16), 2.678 (4.47), 2.691 (3.54), | |
| | 2.701 (5.50), 2.713 (4.47), 2.722 (3.41), 2.743 (2.40), 2.761 (0.73), | |
| | 2.818 (3.32), 2.841 (3.00), 2.864 (1.70), 2.879 (1.45), 2.951 (3.64), | |
| | 2.973 (3.45), 3.449 (3.60), 3.467 (3.34), 3.513 (3.67), 3.530 (3.25), | |
| | 4.039 (0.48), 4.066 (1.61), 4.085 (3.40), 4.099 (5.87), 4.117 (6.43), | |
| | 4.133 (3.11), 6.454 (0.78), 6.482 (13.72), 6.494 (15.14), 6.526 | |
| | (16.00), 6.809 (5.85), 7.038 (3.15), 7.056 (2.76), 8.002 (8.30), 8.591 | |
| | (8.82), 8.595 (8.41), 11.777 (4.24). | |
| Example 949 | 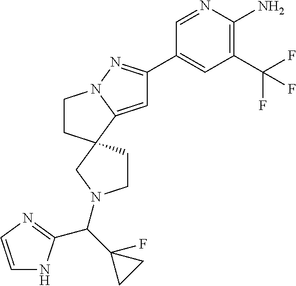 [SMILES: Nc1ncc(-c2cc3n(n2)CC[C@@]32CCN(C(c3ncc[nH]3)C3(F)CC3)C2)cc1C(F)(F)F] | (95% purity, 31% yield) Rₜ = 0.81 min; Instrument: Waters Acquity UPC2 QDA; Column: Chiral Art Amylose-SA 3μ 100 × 4.6 mm; eluent A: CO2; eluent B: 2-propanol + 0.4 vol % diethylamine; isocratic: 30% B; |
| | 5-{(3R)-1-[(1-fluorocyclopropyl)(1H-imidazol-2-yl)methyl]-5′,6′- | gradient: no; |
| | dihydrospiro[pyrrolidine-3,4′-pyrrolo[1,2-b]pyrazol]-2′-yl}-3- | flow: 4 |
| | (trifluoromethyl)pyridin-2-amine (diastereomer 1) | mL/min; |
| | Instrument: Sepiatec: Prep SFC100; Column: Chiral Art Amylose-SA | temperature: |
| | 5μ 250 × 30 mm; eluent A: CO2; eluent B: 2-propanol + 0.4 vol % | 40.0° C.; BPR: |
| | diethylamine; isocratic: 30% B; gradient: no; flow: 100 mL/min; | 1800 psi; UV: |
| | temperature: 40° C.; BPR: 150 bar; UV: 271 nm | 280 nm. |
| | ¹H NMR (400 MHz, DMSO-d6, 22° C.) δ ppm 0.71-0.93 (m, 3 H), | |
| | 1.06-1.17 (m, 1 H), 1.95-2.08 (m, 2 H), 2.59-2.77 (m, 3 H), 2.83 | |
| | (d, 1 H), 2.93-3.01 (m, 1 H), 3.49 (d, 1 H), 4.03-4.15 (m, 2 H), | |
| | 6.48 (s, 1 H), 6.53 (s, 2 H), 6.81 (s, 1 H), 7.06 (s, 1 H), 8.00 (d, 1 H), | |
| | 8.59 (d, 1 H), 11.77 (br s, 1 H). | |
| | [α]²⁰_{D}: +29.8° (c = 1.00, methanol) | |
| Example 950 | 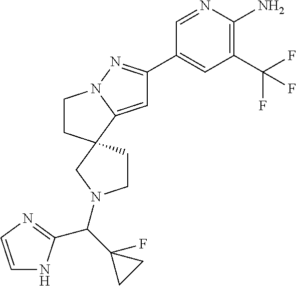 [SMILES: Nc1ncc(-c2cc3n(n2)CC[C@@]32CCN(C(c3ncc[nH]3)C3(F)CC3)C2)cc1C(F)(F)F] | (95% purity, 32% yield) Rₜ = 1.40 min; Instrument: Waters Acquity UPC2 QDA; Column: Chiral Art Amylose-SA 3μ 100 × 4.6 mm; eluent A: CO2; eluent B: 2-propanol + 0.4 vol % diethylamine; isocratic: |
| | 5-{(3R)-1-[(1-fluorocyclopropyl)(1H-imidazol-2-yl)methyl]-5′,6′- | 30% B; |
| | dihydrospiro[pyrrolidine-3,4′-pyrrolo[1,2-b]pyrazol]-2′-yl}-3- | gradient: no; |
| | (trifluoromethyl)pyridin-2-amine (diastereomer 2) | flow: 4 |
| | Instrument: Sepiatec: Prep SFC100; Column: Chiral Art Amylose-SA | mL/min; |
| | 5μ 250 × 30 mm; eluent A: CO2; eluent B: 2-propanol + 0.4 vol % | temperature: |
| | diethylamine; isocratic: 30% B; gradient: no; flow: 100 mL/min; | 40.0° C.; BPR: |
| | temperature: 40° C.; BPR: 150 bar; UV: 271 nm | 1800 psi; UV: |
| | ¹H NMR (400 MHz, DMSO-d₆, 22º C.) δ ppm 0.68-0.81 (m, 2 H), | 280 nm. |
| | 0.83-0.95 (m, 1 H), 1.04-1.17 (m, 1 H), 1.96-2.10 (m, 2 H), 2.57- | |
| | 2.76 (m, 3 H), 2.83-2.91 (m, 1 H), 2.96 (d, 1 H), 3.47 (d, 1 H), 4.12 | |
| | (t, 2 H), 6.49 (s, 1 H), 6.53 (s, 2 H), 6.81 (s, 1 H), 7.04 (d, 1 H), 8.00 | |
| | (d, 1 H), 8.59 (d, 1 H), 11.76 (br s, 1 H). | |
| | [α]²⁰_{D}: +70.9° (c = 1.00, methanol) | |
| Example 951 | 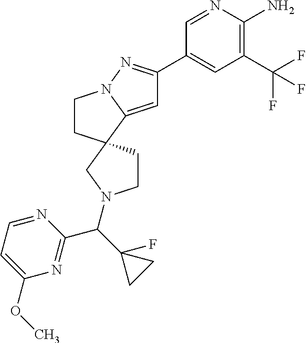 [SMILES: COc1ccnc(C(N2CC[C@@]3(CCn4nc(-c5cnc(N)c(C(F)(F)F)c5)cc43)C2)C2(F)CC2)n1] | (95% purity, 3% yield) LC-MS (Method 1): Rₜ = 1.16 min; MS (ESlpos): m/z = 486 [M + H]⁺ |
| | 5-{(3R)-1-[cyclopropyl(4-methoxypyrimidin-2-yl)methyl]-5′,6′- | |
| | dihydrospiro[pyrrolidine-3,4′-pyrrolo[1,2-b]pyrazol]-2′-yl}-3- | |
| | (trifluoromethyl)pyridin-2-amine (diastereomer 1 and diastereomer 2) | |
| | ¹H-NMR (400 MHz, DMSO-d6) δ [ppm]: 0.090 (0.55), 0.102 (0.84), | |
| | 0.114 (0.89), 0.125 (0.69), 0.309 (0.67), 0.321 (0.81), 0.331 (0.73), | |
| | 0.342 (0.50), 0.407 (0.43), 0.418 (0.59), 0.431 (0.68), 0.442 (0.70), | |
| | 0.455 (0.61), 0.468 (0.44), 0.625 (0.40), 0.636 (0.60), 0.647 (0.78), | |
| | 0.658 (0.74), 0.670 (0.53), 1.230 (0.42), 1.316 (0.46), 1.328 (0.70), | |
| | 1.339 (0.64), 1.349 (0.68), 1.359 (0.42), 1.945 (0.52), 1.959 (0.56), | |
| | 1.979 (0.85), 1.986 (0.76), 1.998 (1.37), 2.004 (1.25), 2.018 (0.93), | |
| | 2.322 (0.58), 2.326 (0.80), 2.332 (0.56), 2.465 (0.90), 2.518 (4.67), | |
| | 2.522 (3.40), 2.558 (0.79), 2.574 (1.12), 2.591 (0.77), 2.606 (0.69), | |
| | 2.635 (1.41), 2.647 (0.82), 2.658 (1.77), 2.664 (1.37), 2.669 (1.93), | |
| | 2.673 (1.47), 2.689 (2.58), 2.694 (1.62), 2.705 (1.75), 2.712 (2.19), | |
| | 2.717 (1.64), 2.729 (1.44), 2.793 (1.32), 2.815 (0.90), 2.917 (0.68), | |
| | 2.933 (0.60), 3.022 (1.32), 3.045 (1.15), 3.128 (0.43), 3.912 (14.09), | |
| | 3.915 (16.00), 4.052 (0.55), 4.071 (0.95), 4.078 (0.90), 4.086 (1.80), | |
| | 4.104 (2.48), 4.121 (1.20), 6.384 (4.81), 6.406 (5.81), 6.516 (5.37), | |
| | 6.805 (5.82), 6.819 (6.21), 7.984 (1.43), 7.989 (1.66), 7.994 (1.91), | |
| | 7.999 (1.83), 8.461 (2.85), 8.469 (3.77), 8.475 (2.70), 8.484 (3.35), | |
| | 8.564 (1.39), 8.570 (2.34), 8.576 (1.70). | |
| Example 952 | 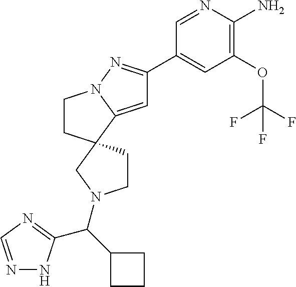 [SMILES: Nc1ncc(-c2cc3n(n2)CC[C@@]32CCN(C(c3ncn[nH]3)C3CCC3)C2)cc1OC(F)(F)F] | (95% purity, 48% yield) LC-MS (Method 1): Rₜ = 0.92 min; MS (ESlpos): m/z = 475 [M + H]⁺ |
| | 5-{(3R)-1-[cyclobutyl(1H-1,2,4-triazol-5-yl)methyl]-5′,6′- | |
| | dihydrospiro[pyrrolidine-3,4′-pyrrolo[1,2-b]pyrazol]-2′-yl}-3- | |
| | (trifluoromethoxy)pyridin-2-amine (diastereomer 1 and diastereomer 2) | |
| | ¹H-NMR (400 MHz, DMSO-d6) δ [ppm]: 1.233 (0.90), 1.474 (1.44), | |
| | 1.496 (2.41), 1.519 (1.96), 1.541 (0.64), 1.693 (2.66), 1.702 (2.67), | |
| | 1.730 (2.25), 1.753 (1.35), 1.781 (1.41), 1.803 (2.16), 1.824 (2.18), | |
| | 1.885 (4.97), 1.906 (4.96), 1.927 (2.61), 2.119 (1.49), 2.322 (1.99), | |
| | 2.326 (2.70), 2.332 (1.92), 2.411 (2.23), 2.518 (10.33), 2.523 (7.02), | |
| | 2.591 (2.66), 2.611 (2.82), 2.637 (5.00), 2.660 (1.67), 2.664 (2.51), | |
| | 2.669 (3.12), 2.673 (2.25), 2.761 (2.20), 2.884 (1.62), 3.748 (2.06), | |
| | 3.773 (2.17), 4.045 (2.70), 4.061 (6.15), 4.073 (6.80), 4.090 (3.07), | |
| | 6.213 (1.56), 6.249 (2.30), 6.470 (16.00), 7.719 (8.21), 7.723 (8.04), | |
| | 7.874 (0.77), 8.297 (10.16), 8.301 (7.15), 8.430 (0.45), 13.785 | |
| | (4.96). | |
| Example 953 | 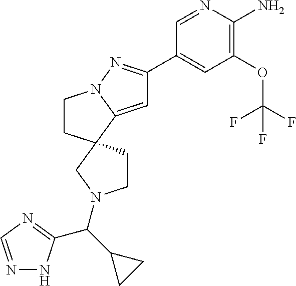 [SMILES: Nc1ncc(-c2cc3n(n2)CC[C@@]32CCN(C(c3ncn[nH]3)C3CC3)C2)cc1OC(F)(F)F] | (95% purity, 33% yield) LC-MS (Method 1): Rₜ = 0.84 min; MS (ESlpos): m/z = 461 [M + H]⁺ |
| | 5-{(3R)-1-[cyclopropyl(1H-1,2,4-triazol-5-yl)methyl]-5′,6′- | |
| | dihydrospiro[pyrrolidine-3,4′-pyrrolo[1,2-b]pyrazol]-2′-yl}-3- | |
| | (trifluoromethoxy)pyridin-2-amine (diastereomer 1 and diastereomer 2) | |
| | ¹H-NMR (400 MHz, DMSO-d6) δ [ppm]: 0.112 (1.51), 0.377 (2.07), | |
| | 0.659 (1.42), 0.851 (0.51), 1.014 (0.47), 1.232 (2.78), 1.993 (3.16), | |
| | 2.322 (2.21), 2.326 (3.05), 2.332 (2.20), 2.518 (16.00), 2.522 | |
| | (10.20), 2.575 (3.14), 2.599 (3.19), 2.622 (2.93), 2.644 (3.26), 2.660 | |
| | (3.06), 2.664 (3.66), 2.669 (4.25), 2.673 (3.42), 2.770 (2.53), 2.794 | |
| | (2.22), 2.851 (1.88), 2.875 (2.73), 2.900 (1.69), 2.947 (1.63), 2.969 | |
| | (1.33), 4.085 (4.37), 4.102 (5.14), 4.119 (2.35), 6.336 (1.04), 6.404 | |
| | (3.18), 6.418 (2.83), 6.480 (12.07), 6.540 (1.10), 7.738 (8.05), 7.839 | |
| | (3.64), 8.326 (6.91), 8.446 (0.97), 13.738 (0.78), 13.827 (2.67). | |
| Example 954 | 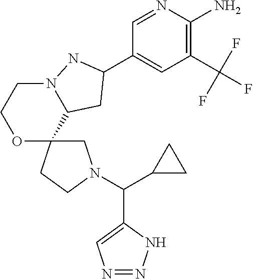 | (95% purity, 44% yield) Rₜ = 2.70 min Instrument: Agilent: 1260, Aurora SFC- Modul; Column: Chiralpak IG 3μ 100 × 4.6 mm; eluent A: CO2; eluent B: 2-propanol + 0.4 vol % diethylamine; isocratic: |
| | 5-{(3′R)-1′-[cyclopropyl(1H-1,2,3-triazol-5-yl)methyl]-6,7- | 25% B; |
| | dihydrospiro[pyrazolo[5,1-c][1,4]oxazine-4,3′-pyrrolidin]-2-yl}-3- | gradient: no; |
| | (trifluoromethyl)pyridin-2-amine (diastereomer 1) | flow: 4 |
| | Instrument: Sepiatec: Prep SFC100; Column: Chiralpak IG 5μ | mL/min; |
| | 250 × 30 mm; eluent A: CO2; eluent B: 2-propanol + 0.4 vol % | temperature: |
| | diethylamine; isocratic: 25% B; gradient: no; flow: 100 mL/min; | 37.5° C.; BPR: |
| | temperature: 40° C.; BPR: 150 bar; UV: 280 nm | 100 bar; UV: |
| | ¹H-NMR (400 MHz, DMSO-d6) δ [ppm]: 0.036 (1.02), 0.048 (1.10), | 280 nm |
| | 0.060 (0.83), 0.327 (0.79), 0.339 (1.01), 0.350 (1.02), 0.372 (0.75), | |
| | 0.386 (0.86), 0.398 (1.02), 0.409 (0.98), 0.659 (0.90), 0.851 (0.32), | |
| | 1.026 (16.00), 1.042 (15.72), 1.137 (0.95), 1.187 (0.92), 1.198 | |
| | (0.90), 1.210 (0.94), 1.231 (1.21), 2.063 (0.33), 2.084 (2.38), 2.097 | |
| | (0.97), 2.115 (1.80), 2.133 (0.77), 2.165 (1.12), 2.180 (1.02), 2.197 | |
| | (0.56), 2.323 (0.92), 2.327 (1.32), 2.331 (0.95), 2.518 (7.72), 2.523 | |
| | (5.01), 2.645 (0.54), 2.664 (2.13), 2.669 (2.11), 2.673 (1.40), 2.684 | |
| | (1.47), 2.703 (0.58), 2.748 (2.45), 2.774 (2.65), 2.808 (3.20), 2.831 | |
| | (3.42), 3.121 (2.53), 3.147 (2.19), 3.749 (0.43), 3.759 (0.43), 3.764 | |
| | (0.58), 3.774 (0.55), 3.779 (0.44), 3.790 (0.41), 4.017 (3.21), 4.028 | |
| | (3.57), 4.060 (3.26), 4.068 (2.94), 4.346 (1.70), 4.356 (1.66), 6.573 | |
| | (6.28), 6.628 (8.61), 7.767 (0.39), 8.035 (3.81), 8.040 (3.86), 8.615 | |
| | (3.57), 8.619 (3.53), 14.664 (0.10), 14.676 (0.10), 14.687 (0.11), | |
| | 14.693 (0.11), 14.696 (0.11), 14.704 (0.11), 14.712 (0.12), 14.716 | |
| | (0.12), 14.719 (0.11), 14.726 (0.11), 14.733 (0.12), 14.741 (0.11), | |
| | 14.746 (0.11), 14.755 (0.11), 14.762 (0.10), 14.771 (0.10). | |
| | [α]²⁰_{D}: +78.8° (c = 1.00, methanol) | |
| Example 955 | 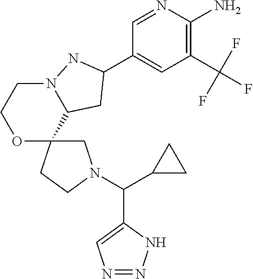 | (95% purity, 26% yield) Rₜ = 3.61 min Instrument: Agilent: 1260, Aurora SFC- Modul; Column: Chiralpak IG 3μ 100 × 4.6 mm; eluent A: CO2; eluent B: 2-propanol + 0.4 vol % diethylamine; isocratic: |
| | 5-{(3′R)-1′-[cyclopropyl(1H-1,2,3-triazol-5-yl)methyl]-6,7- | 25% B; |
| | dihydrospiro[pyrazolo[5,1-c][1,4]oxazine-4,3′-pyrrolidin]-2-yl}-3- | gradient: no; |
| | (trifluoromethyl)pyridin-2-amine (diastereomer 2) | flow: 4 |
| | Instrument: Sepiatec: Prep SFC100; Column: Chiralpak IG 5μ | mL/min; |
| | 250 × 30 mm; eluent A: CO2; eluent B: 2-propanol + 0.4 vol % | temperature: |
| | diethylamine; isocratic: 25% B; gradient: no; flow: 100 ml/min; | 37.5° C.; BPR: |
| | temperature: 40° C.; BPR: 150 bar; UV: 280 nm | 100 bar; UV: |
| | ¹H-NMR (400 MHz, DMSO-d6) δ [ppm]: 0.022 (1.19), 0.032 (1.28), | 280 nm |
| | 0.044 (1.00), 0.332 (0.93), 0.345 (1.18), 0.356 (1.17), 0.396 (1.03), | |
| | 0.407 (1.23), 0.419 (1.16), 0.665 (1.07), 0.851 (0.44), 1.026 (16.00), | |
| | 1.042 (15.74), 1.137 (1.60), 1.231 (2.02), 1.352 (0.32), 1.959 (0.32), | |
| | 2.084 (1.15), 2.100 (1.17), 2.116 (2.03), 2.135 (0.80), 2.156 (0.77), | |
| | 2.173 (1.45), 2.189 (1.36), 2.205 (0.75), 2.323 (1.08), 2.327 (1.52), | |
| | 2.331 (1.11), 2.518 (8.75), 2.523 (5.63), 2.631 (0.65), 2.650 (1.33), | |
| | 2.665 (2.29), 2.669 (2.84), 2.673 (1.99), 2.687 (0.73), 2.830 (3.13), | |
| | 2.853 (2.92), 2.876 (0.63), 2.907 (6.43), 2.938 (1.60), 2.959 (1.26), | |
| | 2.976 (0.57), 3.632 (0.30), 3.749 (0.47), 3.759 (0.45), 3.764 (0.61), | |
| | 3.775 (0.58), 3.779 (0.46), 3.790 (0.45), 3.949 (0.57), 3.966 (1.08), | |
| | 3.981 (2.48), 3.995 (3.39), 4.006 (1.94), 4.027 (1.24), 4.049 (3.19), | |
| | 4.061 (3.82), 4.346 (1.83), 4.356 (1.83), 6.572 (7.24), 6.621 (5.87), | |
| | 7.766 (0.46), 8.037 (4.36), 8.042 (4.41), 8.615 (4.08), 8.619 (4.04), | |
| | 14.707 (0.13), 14.716 (0.13), 14.719 (0.13), 14.722 (0.13), 14.728 | |
| | (0.13), 14.735 (0.13), 14.742 (0.13), 14.748 (0.14), 14.756 (0.13), | |
| | 14.759 (0.13), 14.769 (0.13), 14.771 (0.13). | |
| | [α]²⁰_{D}: +66.8° (c = 1.00, methanol) | |
| Example 956 | 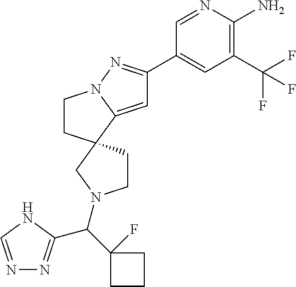 [SMILES: Nc1ncc(-c2cc3n(n2)CC[C@@]32CCN(C(c3nnc[nH]3)C3(F)CCC3)C2)cc1C(F)(F)F] | (90% purity, 1% yield) LC-MS (Method 8): Rₜ = 0.94 min; MS (ESlpos): m/z = 477 [M + H]⁺ |
| | 5-{(3R)-1-[(1-fluorocyclobutyl)(4H-1,2,4-triazol-3-yl)methyl]-5′,6′- | |
| | dihydrospiro[pyrrolidine-3,4′-pyrrolo[1,2-b]pyrazol]-2′-yl}-3- | |
| | (trifluoromethyl)pyridin-2-amine (diastereomer 1 and diastereomer 2) | |
| | ¹H-NMR (400 MHz, METHANOL-d4) δ [ppm]: 0.100 (0.64), 0.885 | |
| | (0.87), 0.903 (1.81), 0.924 (1.23), 0.979 (0.79), 1.221 (1.25), 1.288 | |
| | (5.86), 1.334 (1.94), 1.401 (0.75), 1.541 (0.81), 1.562 (1.55), 1.571 | |
| | (1.50), 1.583 (1.82), 1.593 (1.68), 1.600 (1.40), 1.892 (2.04), 1.900 | |
| | (1.62), 1.908 (1.60), 1.924 (1.47), 1.936 (1.92), 1.952 (1.35), 1.959 | |
| | (1.18), 1.970 (1.81), 1.985 (2.03), 1.990 (1.92), 2.000 (2.15), 2.004 | |
| | (2.15), 2.021 (2.57), 2.031 (2.32), 2.036 (4.97), 2.045 (2.36), 2.052 | |
| | (2.32), 2.066 (2.86), 2.077 (1.28), 2.083 (2.03), 2.088 (1.80), 2.098 | |
| | (1.71), 2.104 (1.60), 2.114 (1.53), 2.126 (2.20), 2.135 (2.12), 2.148 | |
| | (3.37), 2.157 (6.66), 2.174 (2.58), 2.199 (2.14), 2.221 (1.06), 2.254 | |
| | (0.44), 2.323 (0.84), 2.354 (1.37), 2.378 (1.58), 2.401 (1.24), 2.432 | |
| | (0.96), 2.455 (0.51), 2.506 (1.04), 2.525 (2.22), 2.542 (2.93), 2.546 | |
| | (2.45), 2.558 (4.42), 2.574 (3.97), 2.578 (3.15), 2.594 (2.33), 2.610 | |
| | (1.63), 2.621 (2.08), 2.626 (2.17), 2.629 (1.90), 2.635 (2.45), 2.641 | |
| | (2.55), 2.655 (2.57), 2.660 (7.57), 2.668 (1.69), 2.673 (1.40), 2.677 | |
| | (1.06), 2.688 (1.29), 2.701 (0.53), 2.799 (0.42), 2.822 (0.40), 2.855 | |
| | (0.63), 2.871 (2.09), 2.893 (7.00), 2.911 (10.09), 2.939 (2.14), 2.952 | |
| | (2.36), 2.960 (2.91), 2.974 (2.63), 2.982 (1.41), 2.996 (1.00), 3.129 | |
| | (0.59), 3.133 (0.85), 3.137 (0.66), 3.141 (0.47), 3.330 (0.94), 3.334 | |
| | (0.69), 3.478 (0.50), 3.482 (0.88), 3.487 (0.50), 3.762 (0.42), 3.787 | |
| | (0.45), 4.091 (0.54), 4.106 (0.81), 4.110 (0.75), 4.117 (2.75), 4.133 | |
| | (4.91), 4.136 (4.33), 4.146 (5.61), 4.151 (6.47), 4.166 (3.88), 4.169 | |
| | (2.41), 4.178 (1.02), 4.204 (3.13), 4.211 (2.57), 4.275 (3.03), 4.284 | |
| | (2.40), 4.844 (0.68), 5.344 (0.48), 6.249 (16.00), 6.292 (13.22), | |
| | 6.302 (0.89), 8.090 (6.09), 8.094 (8.44), 8.096 (7.44), 8.259 (1.32), | |
| | 8.509 (7.89), 8.511 (7.17), 8.551 (1.29). | |
| Example 957 | 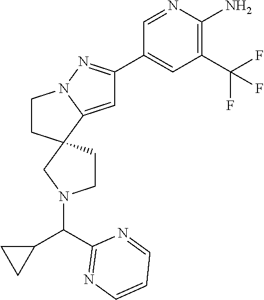 [SMILES: Nc1ncc(-c2cc3n(n2)CC[C@@]32CCN(C(c3ncccn3)C3CC3)C2)cc1C(F)(F)F] | (98% purity, 9% yield) LC-MS (Method 1): Rₜ = 1.02 min; MS (ESlpos): m/z = 456 [M + H]⁺ |
| | 5-{(3R)-1-[cyclopropyl(pyrimidin-2-yl)methyl]-5′,6′- | |
| | dihydrospiro[pyrrolidine-3,4′-pyrrolo[1,2-b]pyrazol]-2′-yl}-3- | |
| | (trifluoromethyl)pyridin-2-amine (diastereomer 1 and diastereomer 2) | |
| | ¹H-NMR (400 MHz, DMSO-d6) δ [ppm]: 0.027 (0.55), 0.037 (0.67), | |
| | 0.049 (0.65), 0.279 (0.56), 0.290 (0.71), 0.301 (0.62), 0.311 (0.45), | |
| | 0.426 (0.46), 0.440 (0.44), 0.450 (0.44), 0.463 (0.40), 0.474 (0.44), | |
| | 0.488 (0.41), 0.651 (0.44), 0.671 (0.58), 0.678 (0.57), 1.203 (0.51), | |
| | 1.220 (0.51), 1.352 (0.56), 1.361 (0.55), 1.372 (0.47), 1.942 (0.46), | |
| | 1.968 (0.70), 1.984 (1.46), 2.001 (1.09), 2.318 (0.85), 2.467 (1.37), | |
| | 2.518 (16.00), 2.523 (10.19), 2.579 (1.32), 2.589 (1.38), 2.601 | |
| | (2.54), 2.622 (1.98), 2.729 (1.38), 2.753 (1.69), 2.759 (1.74), 2.773 | |
| | (0.59), 2.783 (1.33), 2.796 (1.29), 2.819 (0.91), 3.051 (0.42), 3.074 | |
| | (0.45), 3.114 (0.99), 3.137 (0.91), 4.034 (0.45), 4.051 (1.03), 4.069 | |
| | (0.93), 4.084 (0.57), 4.092 (1.17), 4.109 (2.01), 4.127 (0.98), 6.395 | |
| | (4.34), 6.418 (4.63), 6.516 (4.15), 7.386 (1.36), 7.389 (1.39), 7.398 | |
| | (2.76), 7.402 (2.88), 7.410 (1.43), 7.414 (1.53), 7.995 (1.25), 8.001 | |
| | (2.08), 8.007 (1.46), 8.577 (2.26), 8.779 (5.22), 8.785 (5.89), 8.791 | |
| | (5.51), 8.797 (5.17). | |
| Example 958 | 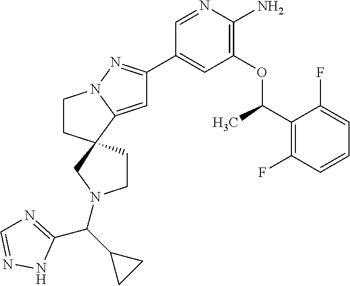 [SMILES: C[C@@H](Oc1cc(-c2cc3n(n2)CC[C@@]32CCN(C(c3ncn[nH]3)C3CC3)C2)cnc1N)c1c(F)cccc1F] | (99% purity, 33% yield) Rₜ = 2.91 min; Instrument: Agilent: 1260, Aurora SFC- Modul; Column: Chiralpak IG 3μ 100 × 4.6 mm; eluent A: CO2; eluent B: methanol + 0.2 vol % aqueous ammonia (32%); |
| | 5-{(3R)-1-[cyclopropyl(1H-1,2,4-triazol-5-yl)methyl]-5′,6′- | isocratic: |
| | dihydrospiro[pyrrolidine-3,4′-pyrrolo[1,2-b]pyrazol]-2′-yl}-3-[(1R)-1- | 40% B; |
| | (2,6-difluorophenyl)ethoxy]pyridin-2-amine (diastereomer 1) | gradient: no; |
| | Instrument: Sepiatec: Prep SFC100; Column: Chiralpak IG 5μ | flow: 4 |
| | 250 × 30 mm; eluent A: CO2; eluent B: methanol + 0.2 vol % aqueous | ml/min; |
| | ammonia (32%); isocratic: 40% B; gradient: no; flow: 100 mL/min; | temperature: |
| | temperature: 40° C.; BPR: 150 bar; UV: 280 nm | 37.5° C.; BPR: |
| | ¹H NMR (400 MHz, DMSO-d6, 22° C.) 0 ppm -0.01-0.15 (m, 1 H) | 100 bar; UV: |
| | 0.28-0.45 (m, 2 H) 0.56-0.71 (m, 1 H) 1.18-1.35 (m, 1 H) 1.75 (d, | 280 nm |
| | 3 H) 1.86-2.07 (m, 2 H) 2.56-2.66 (m, 3 H) 2.73-2.81 (m, 1 H) | |
| | 2.86 (br d, 1 H) 2.93-3.04 (m, 1 H) 4.02-4.14 (m, 2 H) 5.64 (br s, 2 | |
| | H) 5.82 (q, 1 H) 6.11-6.25 (m, 1 H) 7.05-7.15 (m, 2 H) 7.33 (s, 1 | |
| | H) 7.35-7.44 (m, 1 H) 7.80-8.47 (m, 2 H) 13.69-13.89 (m, 1 H). | |
| | [α]²⁰_{D}: +224.2° (c = 1.00, methanol) | |
| Example 959 | 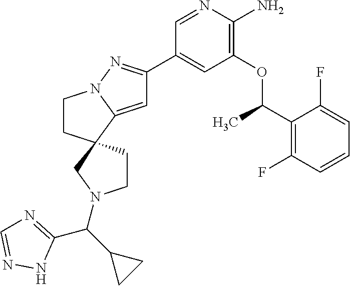 [SMILES: C[C@@H](Oc1cc(-c2cc3n(n2)CC[C@@]32CCN(C(c3ncn[nH]3)C3CC3)C2)cnc1N)c1c(F)cccc1F] | (99% purity, 33% yield) Rₜ = 4.72 min; Instrument: Agilent: 1260, Aurora SFC- Modul; Column: Chiralpak IG 100 × 4.6 mm; eluent A: CO2; eluent B: methanol + 0.2 vol % aqueous ammonia (32%); isocratic: |
| | 5-{(3R)-1-[cyclopropyl(1H-1,2,4-triazol-5-yl)methyl]-5′,6′- | 40% B; |
| | dihydrospiro[pyrrolidine-3,4′-pyrrolo[1,2-b]pyrazol]-2′-yl}-3-[(1R)- 1- | gradient: no; |
| | (2,6-difluorophenyl)ethoxy]pyridin-2-amine (diastereomer 2) | flow: 4 |
| | Instrument: Sepiatec: Prep SFC100; Column: Chiralpak IG 5μ | ml/min; |
| | 250 × 30 mm; eluent A: CO2; eluent B: methanol + 0.2 vol % aqueous | temperature: |
| | ammonia (32%); isocratic: 40% B; gradient: no; flow: 100 mL/min; | 37.5° C.; BPR: |
| | temperature: 40° C.; BPR: 150 bar; UV: 280 nm | 100 bar; UV: |
| | ¹H NMR (400 MHz, DMSO-d6, 22° C.) δ ppm 0.01-0.15 (m, 1 H) | 280 nm |
| | 0.27-0.49 (m, 2 H) 0.57-0.72 (m, 1 H) 1.16-1.37 (m, 1 H) 1.75 (d, | |
| | 3 H) 1.89-2.04 (m, 2 H) 2.62 (br d, 2 H) 2.76 (br d, 1 H) 2.84-3.04 | |
| | (m, 2 H) 3.98-4.12 (m, 2 H) 5.64 (s, 2 H) 5.82 (q, 1 H) 6.10-6.28 | |
| | (m, 1 H) 7.05-7.15 (m, 2 H) 7.32 (s, 1 H) 7.39 (tt, 1 H) 7.82-8.48 | |
| | (m, 2 H) 13.64-13.89 (m, 1 H). | |
| | [α]²⁰_{D}: +221.7° (c = 1.00, methanol) | |
| Example 960 | 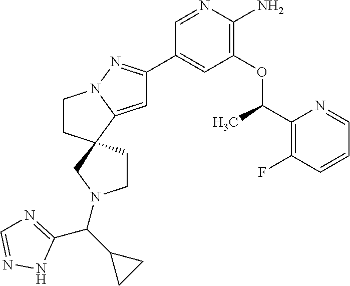 [SMILES: C[C@@H](Oc1cc(-c2cc3n(n2)CC[C@@]32CCN(C(c3ncn[nH]3)C3CC3)C2)cnc1N)c1ncccc1F] | (100% purity, 41% yield) Rₜ = 3.93 min; Instrument: Agilent: 1260, Aurora SFC- Modul; Column: Chiralpak IG 3μ 100 × 4.6 mm; eluent A: CO2; eluent B: methanol + 0.2 vol % aqueous ammonia (32%); |
| | 5-{(3R)-1-[cyclopropyl(1H-1,2,4-triazol-5-yl)methyl]-5′,6′- | isocratic: |
| | dihydrospiro[pyrrolidine-3,4′-pyrrolo[1,2-b]pyrazol]-2′-yl}-3-[(1R)-1-(3- | 40% B; |
| | fluoropyridin-2-yl)ethoxy]pyridin-2-amine (diastereomer 1) | gradient: no; |
| | Instrument: Sepiatec: Prep SFC100; Column: Chiralpak IG 5μ | flow: 4 |
| | 250 × 30 mm; eluent A: CO2; eluent B: methanol + 0.2 vol % aqueous | mL/min; |
| | ammonia (32%); isocratic: 40% B; gradient: no; flow: 100 mL/min; | temperature: |
| | temperature: 40° C.; BPR: 150 bar; UV: 280 nm | 37.5° C.; BPR: |
| | ¹H NMR (400 MHz, DMSO-d6, 22° C.) δ ppm 0.01-0.14 (m, 1 H) | 100 bar; UV: |
| | 0.27-0.45 (m, 2 H) 0.54-0.70 (m, 1 H) 1.19-1.34 (m, 1 H) 1.69 (d, | 280 nm |
| | 3 H) 1.82-2.06 (m, 2 H) 2.55-2.66 (m, 3 H) 2.73-2.81 (m, 1 H) | |
| | 2.86 (br d, 1 H) 2.82-2.89 (m, 1 H) 2.92-3.03 (m, 1 H) 4.03-4.11 | |
| | (m, 2 H) 5.66 (br s, 2 H) 5.78 (q, 1 H) 6.13-6.26 (m, 1 H) 7.32 (d, | |
| | Hz, 1 H) 7.45 (dt, 1 H) 7.74 (ddd, 1 H) 7.84 (s, 1 H) 7.89 (s, 1 H) | |
| | 8.44 (dt, 1 H) 13.70-13.86 (m, 1 H). | |
| | [α]²⁰_{D}: +156.2° (c = 1.00, methanol) | |
| Example 961 | 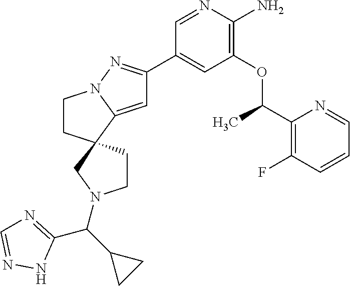 [SMILES: C[C@@H](Oc1cc(-c2cc3n(n2)CC[C@@]32CCN(C(c3ncn[nH]3)C3CC3)C2)cnc1N)c1ncccc1F] | (99% purity, 33% yield) Rₜ = 7.54 min; Instrument: Agilent: 1260, Aurora SFC- Modul; Column: Chiralpak IG 3μ 100 × 4.6 mm; eluent A: CO2; eluent B: methanol + 0.2 vol % aqueous ammonia (32%); |
| | 5-{(3R)-1-[cyclopropyl(1H-1,2,4-triazol-5-yl)methyl]-5′,6′- | isocratic: |
| | dihydrospiro[pyrrolidine-3,4′-pyrrolo[1,2-b]pyrazol]-2′-yl}-3-[(1R)-1-(3- | 40% B; |
| | fluoropyridin-2-yl)ethoxy]pyridin-2-amine (diastereomer 2) | gradient: no; |
| | Instrument: Sepiatec: Prep SFC100; Column: Chiralpak IG 5μ | flow: 4 |
| | 250 × 30 mm; eluent A: CO2; eluent B: methanol + 0.2 vol % aqueous | mL/min; |
| | ammonia (32%); isocratic: 40% B; gradient: no; flow: 100 mL/min; | temperature: |
| | temperature: 40° C.; BPR: 150 bar; UV: 280 nm | 37.5° C.; BPR: |
| | ¹H NMR (400 MHz, DMSO-d₆, 22° C.) δ ppm 0.01-0.14 (m, 1 H) | 100 bar; UV: |
| | 0.29-0.48 (m, 2 H) 0.57-0.71 (m, 1 H) 1.16-1.37 (m, 1 H) 1.69 (d, | 280 nm |
| | 3 H) 1.89-2.05 (m, 2 H) 2.62 (br d, 2 H) 2.75 (br d, 1 H) 2.84-3.03 | |
| | (m, 2 H) 3.97-4.10 (m, 2 H) 5.66 (s, 2 H) 5.77 (q, 1 H) 6.11-6.27 | |
| | (m, 1 H) 7.32 (s, 1 H) 7.45 (dt, 1 H) 7.74 (ddd, 1 H) 7.84 (br s, 1 H) | |
| | 7.89 (s, 1 H) 8.42-8.47 (m, 1 H) 13.70-13.87 (m, 1 H). | |
| | [α]²⁰_{D}: +198.2° (c = 1.00, methanol) | |
| Example 962 | 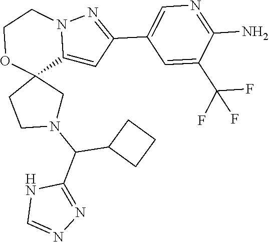 [SMILES: Nc1ncc(-c2cc3n(n2)CCO[C@@]32CCN(C(c3nnc[nH]3)C3CCC3)C2)cc1C(F)(F)F] | 10.5 mg (93% purity) Rₜ = 3.95 min; Instrument: Waters Acquity UPC2 QDA; Column: Chiral Art Amylose-SA 3μ 100 × 4.6 mm; eluent A: CO2; eluent B: 2-propanol |
| | 5-{(3′R)-1′-[cyclobutyl(4H-1,2,4-triazol-3-yl)methyl]-6,7- | + 0.4 vol % |
| | dihydrospiro[pyrazolo[5, 1-c][1,4]oxazine-4,3′-pyrrolidin]-2-yl}-3- | diethylamine; |
| | (trifluoromethyl)pyridin-2-amine (diastereomer 1) | isocratic: |
| | Instrument: Sepiatec: Prep SFC100; Column: Chiral Art Amylose-SA | 20% B; |
| | 5μ 250 × 30 mm; eluent A: CO2; eluent B: 2-propanol + 0.4 vol % | gradient: no; |
| | diethylamine; isocratic: 20% B; gradient: no; flow: 100 mL/min; | flow: 4 |
| | temperature: 40° C.; BPR: 150 bar; UV: 267 nm | mL/min; |
| | ¹H-NMR (400 MHz, DMSO-d6) δ [ppm]: 0.852 (0.69), 1.233 (1.93), | temperature: |
| | 1.483 (0.90), 1.508 (1.33), 1.531 (1.21), 1.691 (1.31), 1.777 (0.81), | 40.0° C.; BPR: |
| | 1.798 (1.27), 1.820 (1.06), 1.876 (1.10), 1.902 (1.38), 1.924 (1.06), | 1800 psi; UV: |
| | 1.990 (1.19), 2.005 (1.20), 2.021 (1.43), 2.081 (1.33), 2.095 (1.58), | 280 nm |
| | 2.114 (1.50), 2.323 (2.87), 2.327 (4.11), 2.332 (2.90), 2.518 (16.00), | |
| | 2.523 (11.75), 2.632 (1.30), 2.646 (1.34), 2.665 (3.49), 2.669 (4.45), | |
| | 2.673 (2.94), 2.787 (1.66), 2.808 (1.42), 2.841 (1.35), 2.867 (2.99), | |
| | 2.897 (4.75), 2.921 (2.68), 3.504 (1.51), 3.719 (2.85), 3.744 (2.65), | |
| | 3.944 (3.43), 3.958 (2.99), 4.032 (3.22), 4.045 (4.21), 6.479 (5.15), | |
| | 6.571 (7.14), 8.010 (4.32), 8.015 (4.37), 8.580 (3.89), 8.585 (3.88), | |
| | 13.782 (0.30), 13.789 (0.34), 13.798 (0.38), 13.804 (0.40), 13.809 | |
| | (0.40), 13.814 (0.39), 13.816 (0.40), 13.818 (0.37), 13.824 (0.34), | |
| | 13.828 (0.30), 13.832 (0.29). | |
| Example 963 | 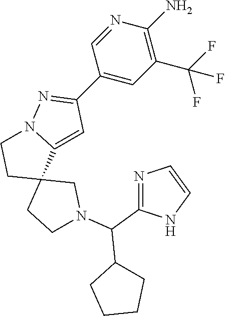 [SMILES: Nc1ncc(-c2cc3n(n2)CC[C@@]32CCN(C(c3ncc[nH]3)C3CCCC3)C2)cc1C(F)(F)F] | (95% purity, 8% yield) Rₜ = 1.22 min; Instrument: Agilent: 1260, Aurora SFC- Modul; Column: Chiralpak IG 3μ 100 × 4.6 mm; eluent A: CO2; eluent B: methanol + 0.2 vol % aqueous ammonia (32%); isocratic: |
| | 5-{(3R)-1-[cyclopentyl(1H-imidazol-2-yl)methyl]-5′,6′- | 30% B; |
| | dihydrospiro[pyrrolidine-3,4′-pyrrolo[1,2-b]pyrazol]-2′-yl}-3- | gradient: no; |
| | (trifluoromethyl)pyridin-2-amine (diastereomer 1) | flow: 4 |
| | Instrument: Sepiatec: Prep SFC100; Column: Chiralpak IG 5μ | mL/min; |
| | 250 × 30 mm; eluent A: CO2; eluent B: methanol + 0.2 vol % aqueous | temperature: |
| | ammonia (32%); isocratic: 30% B; gradient: no; flow: 100 mL/min; | 37.5° C.; BPR: |
| | temperature: 40° C.; BPR: 150 bar; UV: 280 nm | 100 bar; UV: |
| | ¹H-NMR (400 MHz, DMSO-d6) δ [ppm]: −0.073 (0.44), 0.798 (0.75), | 280 nm |
| | 0.814 (0.81), 0.821 (0.82), 0.840 (0.44), 0.904 (0.91), 0.922 (0.69), | |
| | 0.956 (0.90), 0.970 (1.07), 0.990 (0.72), 1.137 (6.62), 1.232 (1.03), | |
| | 1.369 (0.66), 1.387 (1.11), 1.404 (1.45), 1.423 (2.07), 1.433 (2.38), | |
| | 1.454 (2.25), 1.466 (2.51), 1.486 (2.15), 1.504 (2.93), 1.522 (2.02), | |
| | 1.541 (1.07), 1.561 (0.66), 1.780 (1.30), 1.799 (1.25), 1.815 (1.04), | |
| | 1.832 (1.48), 1.846 (1.62), 1.851 (1.64), 1.857 (1.48), 1.865 (1.38), | |
| | 1.877 (1.73), 1.894 (1.27), 1.907 (0.80), 1.925 (0.41), 2.084 (4.07), | |
| | 2.115 (2.92), 2.318 (0.99), 2.323 (2.20), 2.327 (3.17), 2.332 (2.26), | |
| | 2.337 (1.31), 2.356 (1.37), 2.371 (1.47), 2.388 (2.44), 2.406 (1.26), | |
| | 2.425 (1.19), 2.441 (2.55), 2.458 (2.18), 2.518 (11.59), 2.523 (9.08), | |
| | 2.635 (2.83), 2.658 (3.94), 2.665 (3.82), 2.669 (4.69), 2.673 (2.82), | |
| | 2.679 (1.51), 2.685 (1.67), 2.705 (0.68), 2.790 (3.57), 2.813 (3.72), | |
| | 2.830 (1.48), 2.852 (0.54), 3.485 (3.38), 3.511 (3.17), 4.055 (3.27), | |
| | 4.073 (6.74), 4.090 (3.21), 4.562 (1.18), 6.221 (16.00), 6.510 (8.43), | |
| | 6.977 (0.53), 7.975 (4.79), 7.980 (4.84), 8.549 (4.45), 8.553 (4.30), | |
| | 11.786 (0.56). | |
| | [α]²⁰_{D}: +51.5° (c = 1.00, methanol) | |
| Example 964 | 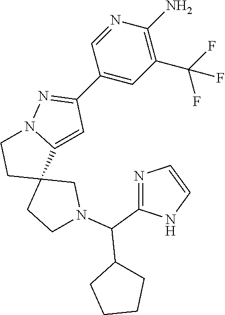 [SMILES: Nc1ncc(-c2cc3n(n2)CC[C@@]32CCN(C(c3ncc[nH]3)C3CCCC3)C2)cc1C(F)(F)F] | (95% purity, 6% yield) Rₜ = 2.40 min; Instrument: Agilent: 1260, Aurora SFC- Modul; Column: Chiralpak IG 3μ 100 × 4.6 mm; eluent A: CO2; eluent B: methanol + 0.2 vol % aqueous ammonia (32%); isocratic: |
| | 5-{(3R)-1-[cyclopentyl(1H-imidazol-2-yl)methyl]-5′,6′- | 30% B; |
| | dihydrospiro[pyrrolidine-3,4′-pyrrolo[1,2-b]pyrazol]-2′-yl}-3- | gradient: no; |
| | (trifluoromethyl)pyridin-2-amine (diastereomer 2) | flow: 4 |
| | Instrument: Sepiatec: Prep SFC100; Column: Chiralpak IG 5μ | mL/min; |
| | 250 × 30 mm; eluent A: CO2; eluent B: methanol + 0.2 vol % aqueous | temperature: |
| | ammonia (32%); isocratic: 30% B.; gradient: no; flow: 100 mL/min; | 37.5° C.; BPR: |
| | temperature: 40° C.; BPR: 150 bar; UV: 280 nm | 100 bar; UV: |
| | ¹H-NMR (400 MHz, DMSO-d6) δ [ppm]: 0.798 (1.26), 0.803 (0.58), | 280 nm |
| | 0.815 (1.39), 0.822 (1.44), 0.840 (0.66), 0.886 (0.77), 0.905 (1.56), | |
| | 0.922 (0.97), 0.932 (0.66), 0.949 (0.88), 0.962 (0.98), 0.980 (0.80), | |
| | 1.071 (0.51), 1.137 (1.32), 1.159 (0.42), 1.232 (0.70), 1.352 (0.75), | |
| | 1.368 (1.04), 1.385 (1.03), 1.398 (0.91), 1.420 (1.20), 1.441 (1.70), | |
| | 1.456 (1.91), 1.471 (1.83), 1.478 (1.68), 1.505 (3.03), 1.520 (2.87), | |
| | 1.820 (0.56), 1.847 (2.95), 1.865 (4.64), 1.882 (2.54), 2.116 (0.52), | |
| | 2.318 (0.79), 2.323 (1.73), 2.327 (2.50), 2.332 (1.78), 2.336 (0.76), | |
| | 2.363 (0.47), 2.381 (0.98), 2.394 (1.48), 2.413 (2.35), 2.432 (1.87), | |
| | 2.449 (2.22), 2.466 (2.52), 2.518 (9.69), 2.523 (7.59), 2.601 (0.61), | |
| | 2.620 (1.56), 2.639 (3.94), 2.661 (4.59), 2.669 (3.03), 2.673 (1.95), | |
| | 2.678 (0.84), 2.732 (3.55), 2.755 (2.36), 2.833 (0.70), 2.850 (1.61), | |
| | 2.866 (1.02), 2.872 (1.25), 2.888 (0.56), 3.485 (3.53), 3.511 (3.28), | |
| | 4.059 (2.44), 4.076 (5.13), 4.094 (2.53), 6.148 (16.00), 6.512 (7.62), | |
| | 6.540 (0.44), 6.563 (0.53), 6.852 (0.49), 7.030 (0.50), 7.970 (4.34), | |
| | 7.976 (4.39), 8.544 (3.99), 8.548 (3.89), 11.762 (1.02). | |
| | [α]²⁰_{D}: +42.4° (c = 1.00, methanol) | |
| Example 965 | 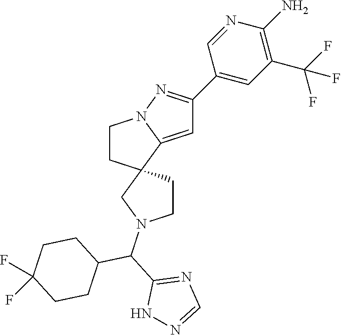 [SMILES: Nc1ncc(-c2cc3n(n2)CC[C@@]32CCN(C(c3ncn[nH]3)C3CCC(F)(F)CC3)C2)cc1C(F)(F)F] | (95% purity, 39% yield) LC-MS (Method 1): R^{t} = 1.01 min; MS (ESlpos): m/z = 524 [M + H]⁺ |
| | 5-{(3R)-1-[(4,4-difluorocyclohexyl)(1H-1,2,4-triazol-5-yl)methyl]-5′,6′- | |
| | dihydrospiro[pyrrolidine-3,4′-pyrrolo[1,2-b]pyrazol]-2′-yl}-3- | |
| | (trifluoromethyl)pyridin-2-amine (diastereomer 1 and diastereomer 2) | |
| | ¹H-NMR (400 MHz, DMSO-d6) δ [ppm]: −0.052 (0.89), 0.852 (0.47), | |
| | 1.044 (0.47), 1.232 (1.71), 1.352 (0.61), 1.787 (0.49), 1.898 (1.64), | |
| | 1.987 (0.51), 2.006 (0.74), 2.024 (0.78), 2.084 (1.04), 2.104 (1.01), | |
| | 2.289 (0.68), 2.318 (0.52), 2.322 (1.16), 2.327 (1.67), 2.332 (1.23), | |
| | 2.336 (0.55), 2.440 (0.77), 2.518 (6.33), 2.523 (4.54), 2.629 (0.70), | |
| | 2.660 (1.08), 2.665 (1.72), 2.669 (2.25), 2.673 (1.80), 2.678 (1.05), | |
| | 2.701 (1.29), 2.839 (1.52), 2.861 (1.46), 3.159 (16.00), 3.172 | |
| | (14.20), 3.642 (0.55), 3.700 (0.44), 4.069 (1.43), 4.082 (3.39), 4.095 | |
| | (4.93), 4.108 (4.60), 4.121 (1.32), 6.207 (0.43), 6.259 (0.52), 6.281 | |
| | (0.47), 6.299 (0.46), 6.452 (0.42), 6.514 (4.46), 7.939 (0.60), 7.983 | |
| | (3.34), 7.988 (3.43), 8.513 (0.62), 8.559 (2.68), 13.849 (1.39). | |
| Example 966 | 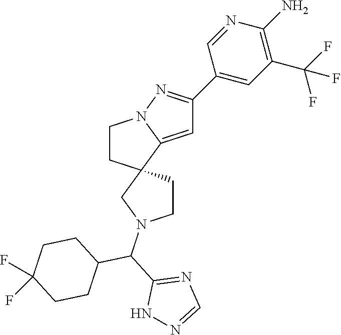 [SMILES: Nc1ncc(-c2cc3n(n2)CC[C@@]32CCN(C(c3ncn[nH]3)C3CCC(F)(F)CC3)C2)cc1C(F)(F)F] | (90% purity, 35% yield) Rₜ = 2.14 min; Instrument: Agilent: 1260, Aurora SFC- Modul; Column: Chiralpak IG 3μ 100 × 4.6 mm; eluent A: CO2; eluent B: methanol + 0.2 vol % aqueous ammonia (32%); isocratic: 20% B; gradient: no; |
| | 5-{(3R)-1-[(4,4-difluorocyclohexyl)(1H-1,2,4-triazol-5-yl)methyl]-5′,6′- | flow: 4 |
| | dihydrospiro[pyrrolidine-3,4′-pyrrolo[1,2-b]pyrazol]-2′-yl}-3- | mL/min; |
| | (trifluoromethyl)pyridin-2-amine (diastereomer 1) | temperature: |
| | Instrument: Sepiatec: Prep SFC100; Column: Chiralpak IG 5μ | 37.5° C.; BPR: |
| | 250 × 30 mm; eluent A: CO2; eluent B: methanol + 0.2 vol % aqueous | 100 bar; UV: |
| | ammonia (32%); isocratic: 20% B; gradient: no; flow: 100 mL/min; | 280 nm |
| | temperature: 40° C.; BPR: 150 bar; UV: 280 nm | |
| | ¹H-NMR (400 MHz, DMSO-d6) δ [ppm]: 1.137 (0.53), 1.232 (0.75), | |
| | 1.358 (0.64), 1.786 (0.53), 1.892 (1.16), 1.906 (1.15), 2.005 (0.50), | |
| | 2.084 (16.00), 2.101 (0.84), 2.115 (0.73), 2.322 (0.86), 2.327 (1.21), | |
| | 2.332 (0.91), 2.336 (0.44), 2.387 (0.46), 2.406 (0.45), 2.422 (0.54), | |
| | 2.436 (0.56), 2.451 (0.56), 2.518 (4.38), 2.523 (3.16), 2.604 (0.50), | |
| | 2.630 (0.55), 2.651 (0.76), 2.660 (0.88), 2.664 (1.36), 2.669 (1.71), | |
| | 2.673 (1.28), 2.678 (0.78), 2.839 (2.40), 2.861 (2.04), 3.628 (0.43), | |
| | 3.651 (0.42), 3.699 (0.44), 4.079 (1.48), 4.091 (1.49), 6.277 (1.04), | |
| | 6.305 (1.10), 6.513 (3.08), 7.943 (0.87), 7.982 (2.88), 7.987 (2.88), | |
| | 8.518 (0.95), 8.556 (2.19), 13.841 (0.91), 13.853 (0.89). | |
| Example 967 | 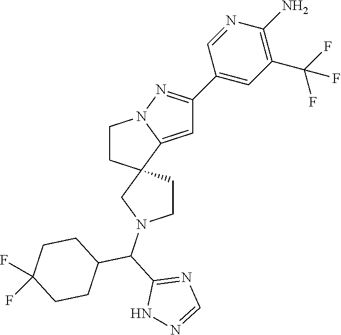 [SMILES: Nc1ncc(-c2cc3n(n2)CC[C@@]32CCN(C(c3ncn[nH]3)C3CCC(F)(F)CC3)C2)cc1C(F)(F)F] | (90% purity, 34% yield) Rₜ = 3.56 min; Instrument: Agilent: 1260, Aurora SFC- Modul; Column: Chiralpak IG 3μ 100 × 4.6 mm; eluent A: CO2; eluent B: methanol + 0.2 vol % aqueous ammonia (32%); isocratic: 20% B; gradient: no; |
| | 5-{(3R)-1-[(4,4-difluorocyclohexyl)(1H-1,2,4-triazol-5-yl)methyl]-5′,6′- | flow: 4 |
| | dihydrospiro[pyrrolidine-3,4′-pyrrolo[1,2-b]pyrazol]-2′-yl}-3- | mL/min; |
| | (trifluoromethyl)pyridin-2-amine (diastereomer 2) | temperature: |
| | Instrument: Sepiatec: Prep SFC100; Column: Chiralpak IG 5μ | 37.5° C.; BPR: |
| | 250 × 30 mm; eluent A: CO2; eluent B: methanol + 0.2 vol % aqueous | 100 bar; UV: |
| | ammonia (32%); isocratic: 20% B; gradient: no; flow: 100 mL/min; | 280 nm |
| | temperature: 40° C.; BPR: 150 bar; UV: 280 nm | |
| | ¹H-NMR (400 MHz, DMSO-d6) δ [ppm]: 1.028 (1.62), 1.043 (2.00), | |
| | 1.232 (2.82), 1.352 (2.35), 1.665 (1.04), 1.780 (1.36), 1.898 (6.37), | |
| | 2.055 (2.97), 2.084 (11.83), 2.109 (3.53), 2.289 (2.38), 2.318 (1.25), | |
| | 2.322 (2.71), 2.327 (3.89), 2.332 (2.82), 2.336 (1.19), 2.439 (3.32), | |
| | 2.518 (15.73), 2.523 (11.23), 2.629 (2.55), 2.660 (2.88), 2.665 | |
| | (4.24), 2.669 (5.66), 2.673 (4.62), 2.678 (2.84), 2.702 (7.42), 2.877 | |
| | (2.55), 3.616 (1.24), 3.641 (1.40), 3.700 (1.52), 4.068 (5.83), 4.085 | |
| | (11.47), 4.102 (5.79), 6.209 (2.55), 6.257 (2.77), 6.452 (1.50), 6.515 | |
| | (16.00), 7.935 (2.34), 7.984 (13.59), 7.989 (13.67), 8.014 (0.48), | |
| | 8.511 (2.20), 8.558 (9.43), 8.594 (0.48), 13.853 (5.72). | |
| Example 968 | 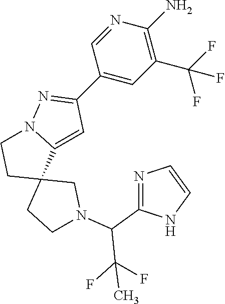 [SMILES: CC(F)(F)C(c1ncc[nH]1)N1CC[C@@]2(CCn3nc(-c4cnc(N)c(C(F)(F)F)c4)cc32)C1] | (99% purity, 1% yield) LC-MS (Method 1): Rₜ = 0.96 min; MS (ESlpos): m/z = 468 [M + H]⁺ |
| | 5-{(3R)-1-[2,2-difluoro-1-(1H-imidazol-2-yl)propyl]-5′,6′- | |
| | dihydrospiro[pyrrolidine-3,4′-pyrrolo[1,2-b]pyrazol]-2′-yl}-3- | |
| | (trifluoromethyl)pyridin-2-amine (diastereomer 1 and diastereomer 2) | |
| | ¹H-NMR (400 MHz, DMSO-d6) δ [ppm]: 1.232 (0.78), 1.352 (0.65), | |
| | 1.752 (5.79), 1.800 (11.16), 1.848 (5.38), 1.878 (0.62), 1.895 (1.50), | |
| | 1.912 (4.58), 1.929 (7.11), 1.947 (3.54), 1.961 (0.76), 2.318 (1.23), | |
| | 2.337 (1.18), 2.392 (0.44), 2.406 (1.63), 2.416 (1.83), 2.424 (3.70), | |
| | 2.433 (3.39), 2.442 (2.74), 2.450 (2.35), 2.455 (2.02), 2.518 (15.86), | |
| | 2.523 (10.67), 2.567 (0.65), 2.678 (1.27), 2.714 (3.15), 2.723 (2.46), | |
| | 2.737 (4.78), 2.745 (6.89), 2.764 (6.58), 2.787 (2.23), 2.862 (0.83), | |
| | 2.882 (1.65), 2.905 (4.32), 2.920 (2.27), 2.928 (3.14), 2.943 (1.40), | |
| | 2.959 (0.54), 4.074 (3.79), 4.089 (7.00), 4.108 (3.62), 4.141 (1.62), | |
| | 4.160 (1.69), 4.168 (1.80), 4.174 (2.01), 4.186 (1.60), 4.193 (1.87), | |
| | 4.201 (1.59), 4.220 (1.22), 6.244 (13.87), 6.344 (16.00), 6.524 | |
| | (13.97), 6.575 (0.67), 6.907 (3.68), 6.910 (6.86), 6.913 (7.32), 6.916 | |
| | (7.41), 6.919 (4.08), 7.111 (3.60), 7.118 (6.81), 7.125 (3.72), 7.978 | |
| | (4.18), 7.983 (4.68), 7.987 (4.96), 7.993 (4.58), 8.555 (3.90), 8.561 | |
| | (4.04), 8.566 (4.42), 8.571 (4.17), 11.969 (2.16), 11.991 (2.23). | |
| Example 969 | 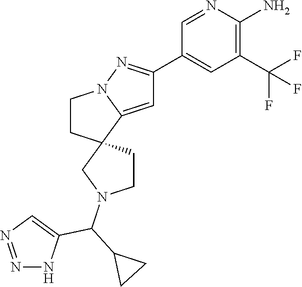 [SMILES: Nc1ncc(-c2cc3n(n2)CC[C@@]32CCN(C(c3cnn[nH]3)C3CC3)C2)cc1C(F)(F)F] | (95% purity, 31% yield) Rₜ = 2.49 min; Instrument: Thermo Fisher UltiMate 3000; Column: YMC Amylose SA 3μ, 100 × 4.6; eluent A: hexane + 0.1 vol % diethylamine; eluent B: ethanol; isocratic: |
| | 5-{(3R)-1-[cyclopropyl(1H-1,2,3-triazol-5-yl)methyl]-5′,6′- | 70% A + 30% B; |
| | dihydrospiro[pyrrolidine-3,4′-pyrrolo[1,2-b]pyrazol]-2′-yl}-3- | flow: 1.4 |
| | (trifluoromethyl)pyridin-2-amine (diastereomer 1) | mL/min; |
| | Instrument: PrepCon Labomatic HPLC-3; Column: YMC Amylose | temperature: |
| | SA 5μ, 250 × 50; eluent A: hexane + 0.1 vol % diethylamine; eluent B: | 25° C.; UV: |
| | ethanol; isocratic: 70% A + 30% B; flow: 60 mL/min; temperature: | 280 nm; |
| | 25° C.; UV: 280 nm; | |
| | ¹H NMR (DMSO-d6, 400 MHz): δ (ppm) 8.59 (d, 1H), 8.01 (d, 1H), | |
| | 7.77 (br s, 1H), 6.51 (s, 2H), 6.42 (s, 1H), 4.02-4.14 (m, 2H), 2.90- | |
| | 2.98 (m, 1H), 2.85 (d, 1H), 2.53-2.73 (m, 4H), 1.93-2.04 (m, 2H), | |
| | 1.14-1.25 (m, 1H), 0.61-0.69 (m, 1H), 0.29-0.45 (m, 2H), 0.04 | |
| | (dq, 1H). | |
| | [α]²⁰_{D}: +43.7° (c = 1.00, methanol) | |
| Example 970 | 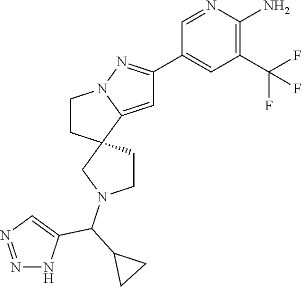 [SMILES: Nc1ncc(-c2cc3n(n2)CC[C@@]32CCN(C(c3cnn[nH]3)C3CC3)C2)cc1C(F)(F)F] | (95% purity, 37% yield) Rₜ = 3.69 min; Instrument: Thermo Fisher UltiMate 3000; Column: YMC Amylose SA 3μ, 100 × 4.6; eluent A: hexane + 0.1 vol % diethylamine; eluent B: ethanol; isocratic: |
| | 5-{(3R)-1-[cyclopropyl(1H-1,2,3-triazol-5-yl)methyl]-5′,6′- | 70% A + 30% B; |
| | dihydrospiro[pyrrolidine-3,4′-pyrrolo[1,2-b]pyrazol]-2′-yl}-3- | flow: 1.4 |
| | (trifluoromethyl)pyridin-2-amine (diastereomer 2) | mL/min; |
| | Instrument: PrepCon Labomatic HPLC-3; Column: YMC Amylose | temperature: |
| | SA 5μ, 250 × 50; eluent A: hexane + 0.1 vol % diethylamine; eluent B: | 25° C.; UV: |
| | ethanol; isocratic: 70% A + 30% B; flow: 60 mL/min; temperature: | 280 nm; |
| | 25° C.; UV: 280 nm; | |
| | ¹H NMR (DMSO-d6, 400 MHz): δ (ppm) 8.59 (d, 1H), 8.01 (d, 1H), | |
| | 7.77 (br s, 1H), 6.51 (s, 2H), 6.42 (s, 1H), 4.02-4.14 (m, 2H), 2.90- | |
| | 2.98 (m, 1H), 2.85 (d, 1H), 2.53-2.73 (m, 4H), 1.93-2.04 (m, 2H), | |
| | 1.14-1.25 (m, 1H), 0.61-0.69 (m, 1H), 0.29-0.45 (m, 2H), 0.04 | |
| | (dq, 1H) | |
| | [α]²⁰_{D}: +43.7° (c = 1.00, methanol) | |
| Example 971 | 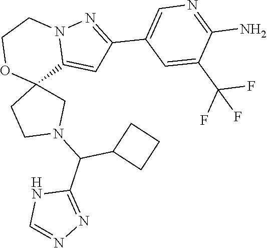 [SMILES: Nc1ncc(-c2cc3n(n2)CCO[C@@]32CCN(C(c3nnc[nH]3)C3CCC3)C2)cc1C(F)(F)F] | (92% purity, 24% yield) LC-MS (Method 1): Rₜ = 0.89 min; MS (ESlpos): m/z = 475 [M + H]⁺ |
| | 5-{(3′R)-1′-[cyclobutyl(4H-1,2,4-triazol-3-yl)methyl]-6,7- | |
| | dihydrospiro[pyrazolo[5,1-c][1,4]oxazine-4,3′-pyrrolidin]-2-yl}-3- | |
| | (trifluoromethyl)pyridin-2-amine (diastereomer 1 and diastereomer 2) | |
| | ¹H-NMR (400 MHz, DMSO-d6) δ [ppm]: 1.154 (4.01), 1.171 (7.72), | |
| | 1.190 (3.90), 1.231 (0.52), 1.502 (1.08), 1.688 (1.28), 1.798 (0.90), | |
| | 1.879 (0.99), 1.902 (1.33), 1.924 (0.83), 1.987 (16.00), 2.084 (1.47), | |
| | 2.327 (1.17), 2.518 (5.03), 2.523 (3.46), 2.629 (1.06), 2.665 (1.13), | |
| | 2.669 (1.38), 2.807 (1.37), 2.818 (1.27), 2.898 (2.21), 3.003 (0.61), | |
| | 3.734 (1.01), 3.959 (3.24), 3.999 (1.33), 4.017 (3.74), 4.034 (6.13), | |
| | 4.052 (4.04), 5.758 (5.87), 6.513 (0.98), 6.570 (6.43), 7.867 (0.65), | |
| | 8.011 (4.80), 8.444 (0.35), 8.456 (0.39), 8.581 (3.31), 13.825 (0.95). | |
| Example 972 | 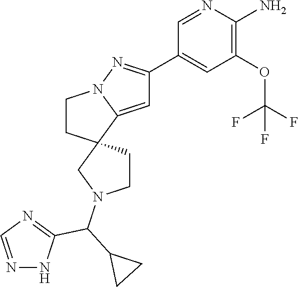 [SMILES: Nc1ncc(-c2cc3n(n2)CC[C@@]32CCN(C(c3ncn[nH]3)C3CC3)C2)cc1OC(F)(F)F] | (95% purity, 44% yield) Rₜ = 3.16 min; Instrument: Thermo Fisher UltiMate 3000; Column: YMC Amylose SA 3μ, 100 × 4.6; eluent A: hexane + 0.1 vol % diethylamine; eluent B: ethanol; isocratic: |
| | 5-{(3R)-1-[cyclopropyl(1H-1,2,4-triazol-5-yl)methyl]-5′,6′- | 80% A + 20% B; |
| | dihydrospiro[pyrrolidine-3,4′-pyrrolo[1,2-b]pyrazol]-2′-yl}-3- | flow: 1.4 |
| | (trifluoromethoxy)pyridin-2-amine (diastereomer 1) | mL/min; |
| | Instrument: PrepCon Labomatic HPLC-3; Column: YMC Amylose | temperature: |
| | SA 5μ, 250 × 30; eluent A: hexane + 0.1 vol % diethylamine; eluent B: | 25° C.; UV: |
| | ethanol; isocratic: 80% A + 20% B; flow: 60 mL/min; temperature: | 280 nm; |
| | 25° C.; UV: 280 nm; | |
| | ¹H NMR (400 MHz, DMSO-d6, 22° C.) δ ppm 0.04-0.13 (m, 1 H) | |
| | 0.29-0.43 (m, 2 H) 0.59-0.68 (m, 1 H) 1.21-1.32 (m, 1 H) 1.89- | |
| | 2.05 (m, 2 H) 2.54-2.68 (m, 3 H) 2.74-2.82 (m, 1 H) 2.85 (d, 1 H) | |
| | 2.98 (br d, 1 H) 4.10 (t, 2 H) 6.38 (s, 1 H) 6.48 (s, 2 H) 7.71-7.76 | |
| | (m, 1 H) 7.79-7.99 (m, 1 H) 8.33 (d, 1 H) 13.79 (br s, 1 H). | |
| | [α]²⁰_{D}: +61.6° (c = 1.00, methanol) | |
| Example 973 | 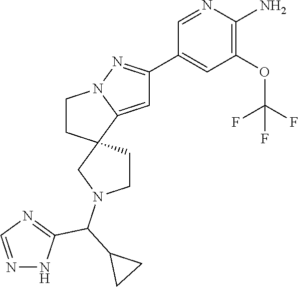 [SMILES: Nc1ncc(-c2cc3n(n2)CC[C@@]32CCN(C(c3ncn[nH]3)C3CC3)C2)cc1OC(F)(F)F] | (95% purity, 46% yield) Rₜ = 4.32 min; Instrument: Thermo Fisher UltiMate 3000; Column: YMC Amylose SA 3μ, 100 × 4.6; eluent A: hexane + 0.1 vol % diethylamine; eluent B: ethanol; isocratic: |
| | 5-{(3R)-1-[cyclopropyl(1H-1,2,4-triazol-5-yl)methyl]-5′,6′- | 80% A + 20% B; |
| | dihydrospiro[pyrrolidine-3,4′-pyrrolo[1,2-b]pyrazol]-2′-yl}-3- | flow: 1.4 |
| | (trifluoromethoxy)pyridin-2-amine (diastereomer 2) | mL/min; |
| | Instrument: PrepCon Labomatic HPLC-3; Column: YMC Amylose | temperature: |
| | SA 5μ, 250 × 30; eluent A: hexane + 0.1 vol % diethylamine; eluent B: | 25° C.; UV: |
| | ethanol; isocratic: 80% A + 20% B; flow: 60 mL/min; temperature: | 280 nm; |
| | 25° C.; UV: 280 nm; | |
| | ¹H NMR (400 MHz, DMSO-d6, 22° C.) δ ppm 0.01-0.15 (m, 1 H) | |
| | 0.28-0.48 (m, 2 H) 0.56-0.72 (m, 1 H) 1.19-1.29 (m, 1 H) 1.93- | |
| | 2.04 (m, 2 H) 2.54-2.68 (m, 3 H) 2.78 (br d, 1 H) 2.84-3.06 (m, 2 | |
| | H) 4.00-4.14 (m, 2 H) 6.30-6.43 (m, 1 H) 6.48 (br s, 2 H) 7.74 (t, 1 | |
| | H) 7.84 (s, 1 H, tautomer 1) 8.33 (br s, 1 H) 8.45 (br s, 1 H tautomer | |
| | 2) 13.70-13.87 (m, 1 H). | |
| | [α]²⁰_{D}: +41.6° (c = 1.00, methanol) | |

### Experimental Section—Biological Assays

Biological In Vitro Assays

The in vitro activity of the compounds of the present invention can be demonstrated in the following assays:

The example testing experiments described herein serve to illustrate the present invention and the invention is not limited to the examples given.

Biological Evaluation

In order that this invention may be better understood, the following examples are set forth. These examples are for the purpose of illustration only, and are not to be construed as limiting the scope of the invention in any manner. All publications mentioned herein are incorporated by reference in their entirety.

Demonstration of the activity of the compounds of the present invention may be accomplished through in vitro and in vivo assays that are well known in the art. For example, to demonstrate the efficacy of a pharmaceutical agent to inhibit and be selective against something the following assays may be used.

Binding Competition Assay

The ability of the compounds of the present invention to inhibit the binding of an Alexa647-labelled ATP-competitive kinase inhibitor to a Glutathione-S-transferase- (GST-) fusion protein was quantified employing the TR-FRET-based binding competition assay as described in the following paragraphs. A recombinant fusion protein of N-terminal GST and full-length human, expressed by baculovirus infected SF9 insect cells and purified by Glutathione Sepharose affinity chromatography, was used as GST-fusion protein. Tracer 222 from Invitrogen (catalogue no. PR9198A) was used as Alexa647-labelled ATP-competitive kinase inhibitor.

For the assay 50 nl of a 100 fold concentrated solution of the test compound in DMSO was pipetted into either a black low volume 384 well microtiter plate or a black 1536 well microtiter plate (both Greiner Bio-One, Frickenhausen, Germany), 3 μL solution of Tracer 222 (25 nM=>final concentration in 5 μL assay volume is 15 nM) in aqueous assay buffer [25 mM Tris/HCl pH 7.5, 10 mM MgCl₂, 5 mM β-glycerolphosphate, 2.5 mM dithiothreitol, 0.5 mM ethylene glycol-bis(2-aminoethylether)-N,N,N′,N′-tetraacetic acid [EGTA], 0.5 mM sodium ortho-vanadate, 0.01% (w/v) bovine serum albumin [BSA], 0.005% (w/v) Pluronic F-127 (Sigma)] were added. Then the binding competition was started by the addition of 2 μL of a solution of the GST-fusion protein (2.5 nM=>final conc. in the 5 μL assay volume is 1 nM) and of Anti-GST-Tb (1.25 nM=>final conc. in the 5 μL assay volume is 0.5 nM), a Lumi4®-Tb Cryptate-conjugated anti-GST-antibody from Cisbio Bioassays (France), in assay buffer.

The resulting mixture was incubated 30 min at 22° C. to allow the formation of a complex between the Tracer 222, the fusion protein and Anti-GST-Tb. Subsequently the amount of this complex was evaluated by measurement of the resonance energy transfer from the Tb-cryptate to the Tracer 222. Therefore, the fluorescence emissions at 620 nm and 665 nm after excitation at 350 nm were measured in a TR-FRET reader, e.g. a Pherastar (BMG Labtechnologies, Offenburg, Germany) or a Viewlux (Perkin-Elmer). The ratio of the emissions at 665 nm and at 622 nm was taken as the measure for the amount of the complex. The data were normalised (assay reaction without inhibitor=0% inhibition, all other assay components but GST-fusion protein=100% inhibition). Usually the test compounds were tested on the same microtiter plate in 11 different concentrations in the range of 20 μM to 0.07 nM (20 μM, 5.7 μM, 1.6 μM, 0.47 μM, 0.13 μM, 38 nM, 11 nM, 3.1 nM, 0.9 nM, 0.25 nM and 0.07 nM, the dilution series prepared separately before the assay on the level of the 100 fold concentrated solutions in DMSO by serial dilutions, exact concentrations may vary depending pipettors used) in duplicate values for each concentration and IC50 values were calculated using Genedata Screener™ software.

TABLE 1
Measured IC₅₀ values of compounds
regarding inhibition of MAP4K1 (HPK1):

| Example | MAP4K1 IC₅₀ |
|---|---|
| 1 | 2.50E−09 |
| 2 | 3.05E−09 |
| 3 | 3.09E−09 |
| 4 | 3.83E−09 |
| 5 | 3.99E−09 |
| 6 | 4.31E−09 |
| 7 | 4.81E−09 |
| 8 | 5.12E−09 |
| 9 | 5.27E−09 |
| 10 | 5.37E−09 |
| 11 | 5.39E−09 |
| 12 | 5.40E−09 |
| 13 | 5.68E−09 |
| 14 | 3.92E−09 |
| 15 | 2.11E−09 |
| 16 | 3.13E−09 |
| 17 | 3.24E−09 |
| 18 | 3.69E−09 |
| 19 | 4.04E−09 |
| 20 | 4.10E−09 |
| 21 | 4.24E−09 |
| 22 | 4.52E−09 |
| 23 | 4.55E−09 |
| 24 | 4.61E−09 |
| 25 | 4.66E−09 |
| 26 | 4.96E−09 |
| 27 | 5.05E−09 |
| 28 | 5.07E−09 |
| 29 | 5.10E−09 |
| 30 | 5.53E−09 |
| 31 | 4.44E−09 |
| 32 | 5.75E−09 |
| 33 | 6.11E−09 |
| 34 | 6.39E−09 |
| 35 | 6.45E−09 |
| 36 | 6.62E−09 |
| 37 | 6.83E−09 |
| 38 | 7.03E−09 |
| 39 | 7.61E−09 |
| 40 | 8.84E−09 |
| 41 | 9.14E−09 |
| 42 | 9.52E−09 |
| 43 | 1.02E−08 |
| 44 | 1.14E−08 |
| 45 | 1.15E−08 |
| 46 | 1.18E−08 |
| 47 | 1.18E−08 |
| 48 | 1.25E−08 |
| 49 | 1.31E−08 |
| 50 | 1.44E−08 |
| 51 | 1.47E−08 |
| 52 | 1.57E−08 |
| 53 | 1.76E−08 |
| 54 | 1.94E−08 |
| 55 | 2.13E−08 |
| 56 | 2.14E−08 |
| 57 | 2.24E−08 |
| 58 | 2.28E−08 |
| 59 | 2.38E−08 |
| 60 | 2.47E−08 |
| 61 | 2.93E−08 |
| 62 | 2.99E−08 |
| 63 | 3.16E−08 |
| 64 | 3.65E−08 |
| 65 | 3.81E−08 |
| 66 | 4.53E−08 |
| 67 | 4.63E−08 |
| 68 | 4.83E−08 |
| 69 | 5.74E−08 |
| 70 | 5.89E−08 |
| 71 | 6.21E−08 |
| 72 | 6.37E−08 |
| 73 | 9.50E−08 |
| 74 | 9.77E−08 |
| 75 | 1.02E−07 |
| 76 | 1.12E−07 |
| 77 | 1.19E−07 |
| 78 | 1.20E−07 |
| 79 | 1.48E−07 |
| 80 | 1.59E−07 |
| 81 | 1.99E−07 |
| 82 | 2.09E−07 |
| 83 | 2.91E−07 |
| 84 | 5.17E−06 |
| 85 | 5.74E−09 |
| 86 | 6.61E−09 |
| 87 | 7.46E−09 |
| 88 | 1.01E−08 |
| 89 | 1.17E−08 |
| 90 | 4.13E−08 |
| 91 | 4.87E−08 |
| 92 | 1.72E−07 |
| 93 | 2.52E−07 |
| 94 | 7.42E−08 |
| 95 | 2.59E−08 |
| 96 | 4.48E−08 |
| 97 | 6.24E−08 |
| 98 | 7.63E−08 |
| 99 | 5.17E−08 |
| 100 | 1.17E−08 |
| 101 | 1.98E−08 |
| 102 | 3.47E−08 |
| 103 | 1.22E−08 |
| 104 | 5.17E−08 |
| 105 | 2.83E−07 |
| 106 | 9.32E−08 |
| 107 | 1.34E−07 |
| 108 | 1.09E−07 |
| 109 | 6.69E−08 |
| 110 | 6.91E−08 |
| 111 | 5.95E−09 |
| 112 | 6.72E−09 |
| 113 | 1.53E−07 |
| 114 | 1.75E−07 |
| 115 | 1.17E−07 |
| 116 | 8.26E−08 |
| 117 | 1.13E−08 |
| 118 | 1.72E−08 |
| 119 | 1.83E−08 |
| 120 | 6.72E−08 |
| 121 | 7.94E−09 |
| 122 | 2.16E−08 |
| 123 | 5.70E−08 |
| 124 | 1.11E−07 |
| 125 | 4.07E−08 |
| 126 | 6.32E−08 |
| 127 | 2.67E−08 |
| 128 | 7.02E−08 |
| 129 | 3.60E−08 |
| 130 | 4.95E−08 |
| 131 | 9.66E−08 |
| 132 | 3.90E−08 |
| 133 | 3.51E−08 |
| 134 | 2.60E−08 |
| 135 | 1.05E−07 |
| 136 | 5.93E−09 |
| 137 | 2.25E−08 |
| 138 | 2.00E−08 |
| 139 | 4.83E−07 |
| 140 | 1.54E−07 |
| 141 | 2.34E−07 |
| 142 | 2.66E−08 |
| 143 | 1.76E−08 |
| 144 | 7.65E−08 |
| 145 | 3.02E−08 |
| 146 | 2.79E−08 |
| 147 | 1.65E−08 |
| 148 | 2.56E−08 |
| 149 | 1.14E−07 |
| 150 | 1.33E−07 |
| 151 | 6.59E−09 |
| 152 | 3.26E−09 |
| 153 | 1.75E−08 |
| 154 | 1.14E−08 |
| 155 | 5.96E−09 |
| 156 | 7.27E−09 |
| 157 | 1.02E−08 |
| 158 | 1.07E−08 |
| 159 | 6.24E−09 |
| 160 | 6.83E−09 |
| 161 | 2.87E−08 |
| 162 | 4.03E−08 |
| 163 | 3.08E−08 |
| 164 | 6.92E−08 |
| 165 | 9.02E−09 |
| 166 | 6.25E−09 |
| 167 | 1.10E−08 |
| 168 | 2.50E−08 |
| 169 | 1.29E−07 |
| 170 | 7.72E−08 |
| 171 | 4.36E−08 |
| 172 | 7.76E−08 |
| 173 | 1.94E−07 |
| 174 | 8.42E−08 |
| 175 | 3.78E−08 |
| 176 | 3.57E−08 |
| 177 | 3.79E−08 |
| 178 | 8.09E−08 |
| 179 | 8.61E−09 |
| 180 | 2.73E−08 |
| 181 | 3.57E−08 |
| 182 | 2.09E−08 |
| 183 | 7.21E−08 |
| 184 | 4.08E−08 |
| 185 | 2.30E−08 |
| 186 | 3.72E−08 |
| 187 | 5.66E−08 |
| 188 | 7.45E−08 |
| 189 | 2.19E−08 |
| 190 | 3.09E−08 |
| 191 | 3.78E−08 |
| 192 | 1.46E−08 |
| 193 | 3.25E−08 |
| 194 | 3.12E−09 |
| 195 | 2.03E−07 |
| 196 | 1.12E−07 |
| 197 | 1.14E−07 |
| 198 | 2.83E−08 |
| 199 | 2.25E−08 |
| 200 | 9.32E−08 |
| 201 | 1.01E−08 |
| 202 | 2.73E−08 |
| 203 | 2.43E−08 |
| 204 | 1.37E−08 |
| 205 | 1.50E−08 |
| 206 | 3.13E−09 |
| 207 | 1.28E−08 |
| 208 | 1.15E−08 |
| 209 | 2.11E−09 |
| 210 | 4.24E−09 |
| 211 | 5.07E−09 |
| 212 | 1.12E−08 |
| 213 | 1.22E−08 |
| 214 | 2.39E−09 |
| 215 | 1.42E−09 |
| 216 | 6.96E−07 |
| 217 | 1.02E−07 |
| 218 | 4.70E−08 |
| 219 | 5.90E−08 |
| 220 | 1.25E−07 |
| 221 | 3.90E−08 |
| 222 | 3.82E−08 |
| 223 | 5.01E−08 |
| 224 | 1.33E−08 |
| 225 | 1.08E−08 |
| 226 | 7.13E−09 |
| 227 | 2.25E−08 |
| 228 | 2.75E−08 |
| 229 | 4.10E−09 |
| 230 | 4.52E−09 |
| 231 | 3.69E−09 |
| 232 | 4.96E−09 |
| 233 | 5.05E−09 |
| 234 | 2.62E−07 |
| 235 | 9.03E−09 |
| 236 | 4.51E−08 |
| 237 | 4.05E−08 |
| 238 | 3.58E−08 |
| 239 | 3.38E−08 |
| 240 | 1.25E−08 |
| 241 | 1.79E−08 |
| 242 | 1.71E−08 |
| 243 | 8.97E−09 |
| 244 | 1.30E−08 |
| 245 | 6.11E−08 |
| 246 | 6.95E−09 |
| 247 | 2.18E−08 |
| 248 | 1.50E−08 |
| 249 | 5.08E−09 |
| 250 | 5.00E−08 |
| 251 | 5.06E−09 |
| 252 | 7.82E−09 |
| 253 | 1.21E−08 |
| 254 | 1.32E−08 |
| 255 | 1.19E−08 |
| 256 | 1.69E−08 |
| 257 | 1.38E−08 |
| 258 | 1.14E−08 |
| 259 | 1.28E−08 |
| 260 | 7.77E−09 |
| 261 | 7.33E−09 |
| 262 | 5.60E−09 |
| 263 | 3.69E−09 |
| 264 | 1.27E−08 |
| 265 | 4.91E−09 |
| 266 | 4.23E−08 |
| 267 | 3.68E−08 |
| 268 | 3.16E−09 |
| 269 | 4.31E−08 |
| 270 | 2.44E−08 |
| 271 | 6.13E−09 |
| 272 | 5.16E−08 |
| 273 | 6.97E−08 |
| 274 | 1.96E−08 |
| 275 | 1.81E−08 |
| 276 | 4.94E−09 |
| 277 | 5.86E−09 |
| 278 | 2.91E−08 |
| 279 | 6.56E−09 |
| 280 | 6.21E−09 |
| 281 | 4.26E−08 |
| 282 | 4.48E−08 |
| 283 | 1.01E−08 |
| 284 | 2.97E−08 |
| 285 | 6.74E−08 |
| 286 | 2.14E−08 |
| 287 | 1.07E−08 |
| 288 | 1.45E−08 |
| 289 | 1.13E−08 |
| 290 | 6.71E−09 |
| 291 | 5.80E−09 |
| 292 | 3.67E−08 |
| 293 | 4.64E−08 |
| 294 | 7.64E−08 |
| 295 | 3.75E−08 |
| 296 | 1.58E−08 |
| 297 | 4.18E−09 |
| 298 | 2.46E−08 |
| 299 | 2.06E−09 |
| 300 | 4.31E−08 |
| 301 | 1.50E−08 |
| 302 | 3.71E−09 |
| 303 | 2.92E−09 |
| 304 | 3.50E−09 |
| 305 | 3.54E−09 |
| 306 | 1.03E−08 |
| 307 | 1.82E−07 |
| 308 | 4.54E−09 |
| 309 | 5.53E−09 |
| 310 | 7.30E−08 |
| 311 | 8.02E−08 |
| 312 | 4.71E−08 |
| 313 | 9.63E−09 |
| 314 | 1.07E−08 |
| 315 | 2.24E−07 |
| 316 | 3.12E−08 |
| 317 | 7.78E−08 |
| 318 | 2.33E−08 |
| 319 | 4.44E−08 |
| 320 | 8.63E−09 |
| 321 | 5.34E−09 |
| 322 | 9.54E−08 |
| 323 | 4.67E−08 |
| 324 | 6.13E−08 |
| 325 | 3.99E−08 |
| 326 | 1.81E−08 |
| 327 | 1.08E−07 |
| 328 | 9.89E−09 |
| 329 | 1.78E−08 |
| 330 | 1.03E−07 |
| 331 | 1.91E−08 |
| 332 | 1.80E−08 |
| 333 | 4.27E−08 |
| 334 | 1.20E−08 |
| 335 | 6.34E−09 |
| 336 | 6.30E−09 |
| 337 | 7.26E−09 |
| 338 | 9.61E−09 |
| 339 | 6.03E−09 |
| 340 | 1.87E−08 |
| 341 | 1.34E−08 |
| 342 | 7.69E−09 |
| 343 | 1.97E−07 |
| 344 | 1.39E−07 |
| 345 | 5.01E−09 |
| 346 | 3.98E−08 |
| 347 | 9.42E−09 |
| 348 | 8.80E−08 |
| 349 | 2.89E−08 |
| 350 | 3.27E−08 |
| 351 | 2.69E−08 |
| 352 | 1.55E−07 |
| 353 | 3.34E−08 |
| 354 | 3.45E−07 |
| 355 | 3.27E−07 |
| 356 | 4.07E−08 |
| 357 | 2.13E−07 |
| 358 | 1.73E−08 |
| 359 | 2.58E−08 |
| 360 | 2.04E−08 |
| 361 | 6.22E−09 |
| 362 | 5.36E−09 |
| 363 | 1.44E−08 |
| 364 | 2.14E−08 |
| 365 | 8.91E−09 |
| 366 | 1.41E−08 |
| 367 | 3.66E−07 |
| 368 | 1.23E−08 |
| 369 | 5.00E−07 |
| 370 | 1.78E−08 |
| 371 | 1.81E−07 |
| 372 | 4.83E−07 |
| 373 | 5.47E−08 |
| 374 | 4.65E−08 |
| 375 | 5.00E−08 |
| 376 | 2.50E−09 |
| 377 | 3.05E−09 |
| 378 | 3.09E−09 |
| 379 | 3.83E−09 |
| 380 | 3.99E−09 |
| 381 | 4.31E−09 |
| 382 | 4.81E−09 |
| 383 | 5.12E−09 |
| 384 | 5.27E−09 |
| 385 | 5.37E−09 |
| 386 | 5.39E−09 |
| 387 | 5.40E−09 |
| 388 | 5.68E−09 |
| 389 | 4.04E−09 |
| 390 | 4.55E−09 |
| 391 | 4.61E−09 |
| 392 | 5.10E−09 |
| 393 | 5.53E−09 |
| 394 | 8.67E−09 |
| 395 | 3.31E−09 |
| 396 | 4.24E−09 |
| 397 | 3.22E−09 |
| 398 | |
| 399 | 9.63E−09 |
| 400 | 9.89E−09 |
| 401 | 5.98E−09 |
| 402 | 1.33E−07 |
| 403 | 5.88E−09 |
| 404 | 6.07E−09 |
| 405 | 5.49E−07 |
| 406 | 7.11E−09 |
| 407 | 8.12E−09 |
| 408 | 8.95E−09 |
| 409 | 1.37E−08 |
| 410 | 1.44E−08 |
| 411 | 1.45E−08 |
| 412 | 2.04E−07 |
| 413 | 2.84E−07 |
| 414 | 6.57E−07 |
| 415 | 3.37E−08 |
| 416 | 3.99E−08 |
| 417 | 6.70E−08 |
| 418 | 4.38E−08 |
| 419 | 1.34E−07 |
| 420 | 3.29E−08 |
| 421 | 1.59E−07 |
| 422 | 3.29E−08 |
| 423 | 1.50E−07 |
| 424 | 5.28E−08 |
| 425 | 1.55E−07 |
| 426 | 4.10E−07 |
| 427 | 2.35E−07 |
| 428 | 4.66E−09 |
| 429 | 3.08E−08 |
| 430 | 2.04E−08 |
| 431 | 1.68E−08 |
| 432 | 4.04E−08 |
| 433 | 2.81E−08 |
| 434 | 2.15E−08 |
| 435 | 2.27E−08 |
| 436 | 2.61E−08 |
| 437 | 1.48E−08 |
| 438 | 1.18E−08 |
| 439 | 1.40E−08 |
| 440 | 1.76E−07 |
| 441 | 1.01E−07 |
| 442 | 7.49E−08 |
| 443 | 2.90E−08 |
| 444 | 2.07E−08 |
| 445 | 4.21E−08 |
| 446 | 6.27E−08 |
| 447 | 1.01E−06 |
| 448 | 1.57E−06 |
| 449 | 9.02E−07 |
| 450 | 1.36E−08 |
| 451 | 2.10E−07 |
| 452 | 5.99E−07 |
| 453 | 1.34E−06 |
| 454 | 6.28E−07 |
| 455 | 2.78E−06 |
| 456 | 8.00E−07 |
| 457 | 5.34E−07 |
| 458 | 1.78E−06 |
| 459 | 3.32E−07 |
| 460 | 2.47E−09 |
| 461 | 1.11E−07 |
| 462 | 1.08E−07 |
| 463 | 1.11E−07 |
| 464 | 8.80E−08 |
| 465 | 2.00E−07 |
| 466 | 1.01E−06 |
| 467 | 1.29E−07 |
| 468 | 1.59E−08 |
| 469 | 1.11E−07 |
| 470 | 7.65E−06 |
| 471 | 4.10E−08 |
| 472 | 8.82E−08 |
| 473 | 1.59E−07 |
| 474 | 2.16E−08 |
| 475 | 8.67E−08 |
| 476 | 4.48E−08 |
| 477 | 5.74E−07 |
| 478 | 2.13E−07 |
| 479 | 9.43E−08 |
| 480 | 6.09E−08 |
| 481 | 1.46E−07 |
| 482 | 1.68E−07 |
| 483 | 1.02E−06 |
| 484 | 1.01E−07 |
| 485 | 3.19E−07 |
| 486 | 4.76E−08 |
| 487 | 3.05E−07 |
| 488 | 1.05E−07 |
| 489 | 9.35E−09 |
| 490 | 1.41E−07 |
| 491 | 3.81E−07 |
| 492 | 7.89E−08 |
| 493 | 5.82E−07 |
| 494 | 8.85E−08 |
| 495 | 6.87E−08 |
| 496 | 4.93E−08 |
| 497 | 1.51E−08 |
| 498 | 5.27E−08 |
| 499 | 3.41E−08 |
| 500 | 4.79E−08 |
| 501 | 5.91E−08 |
| 502 | 3.75E−08 |
| 503 | 1.64E−07 |
| 504 | 2.34E−08 |
| 505 | 2.84E−08 |
| 506 | 7.71E−08 |
| 507 | 7.45E−08 |
| 508 | 1.08E−08 |
| 509 | 2.86E−07 |
| 510 | 2.04E−07 |
| 511 | 3.91E−07 |
| 512 | 1.81E−07 |
| 513 | 1.26E−08 |
| 514 | 5.75E−07 |
| 515 | 4.00E−08 |
| 516 | 2.64E−07 |
| 517 | 2.25E−07 |
| 518 | 2.71E−08 |
| 519 | 1.81E−07 |
| 520 | 9.60E−08 |
| 521 | 5.58E−07 |
| 522 | 3.35E−07 |
| 523 | 2.05E−08 |
| 524 | 5.67E−08 |
| 525 | 1.47E−06 |
| 526 | 6.93E−09 |
| 527 | 8.30E−08 |
| 528 | 1.34E−06 |
| 529 | 7.32E−07 |
| 530 | 2.25E−07 |
| 531 | 1.14E−07 |
| 532 | 5.60E−07 |
| 533 | ! 2.00E−5 |
| 534 | 2.56E−07 |
| 535 | 9.51E−08 |
| 536 | 1.86E−07 |
| 537 | 4.39E−07 |
| 538 | 1.21E−07 |
| 539 | 8.24E−08 |
| 540 | 1.83E−07 |
| 541 | 2.12E−06 |
| 542 | 7.78E−08 |
| 543 | 1.28E−06 |
| 544 | 2.57E−07 |
| 545 | 2.92E−08 |
| 546 | 2.42E−07 |
| 547 | 3.92E−09 |
| 548 | 1.08E−08 |
| 549 | 5.72E−09 |
| 550 | 1.02E−08 |
| 551 | 1.31E−08 |
| 552 | 4.96E−08 |
| 553 | 8.51E−08 |
| 554 | 4.13E−06 |
| 555 | 5.16E−08 |
| 556 | 4.52E−07 |
| 557 | 3.15E−07 |
| 558 | 5.90E−08 |
| 559 | 1.70E−08 |
| 560 | 1.29E−07 |
| 561 | 5.58E−08 |
| 562 | 9.84E−08 |
| 563 | 3.69E−08 |
| 564 | 7.61E−08 |
| 565 | 5.15E−08 |
| 566 | 2.86E−07 |
| 567 | 4.53E−08 |
| 568 | 1.35E−07 |
| 569 | 3.48E−07 |
| 570 | 1.29E−08 |
| 571 | 8.02E−08 |
| 572 | 1.42E−07 |
| 573 | 2.08E−07 |
| 574 | 2.00E−07 |
| 575 | 2.39E−07 |
| 576 | 4.85E−08 |
| 577 | 1.28E−08 |
| 578 | 9.97E−09 |
| 579 | 1.34E−07 |
| 580 | 1.40E−07 |
| 581 | 6.28E−08 |
| 582 | 1.87E−07 |
| 583 | 1.36E−07 |
| 584 | 2.12E−07 |
| 585 | 5.69E−08 |
| 586 | 3.93E−07 |
| 587 | 1.92E−07 |
| 588 | 1.72E−07 |
| 589 | 6.49E−09 |
| 590 | 4.09E−08 |
| 591 | 3.12E−09 |
| 592 | 7.02E−09 |
| 593 | 3.26E−08 |
| 594 | 3.97E−09 |
| 595 | 1.17E−07 |
| 596 | 6.53E−08 |
| 597 | 1.89E−06 |
| 598 | 2.66E−07 |
| 599 | 7.10E−08 |
| 600 | 1.99E−07 |
| 601 | 1.60E−07 |
| 602 | 1.29E−06 |
| 603 | 6.97E−07 |
| 604 | 5.82E−07 |
| 605 | 1.33E−07 |
| 606 | 6.09E−07 |
| 607 | 1.14E−07 |
| 608 | 3.16E−07 |
| 609 | 7.44E−07 |
| 610 | 3.30E−07 |
| 611 | 8.17E−06 |
| 612 | 5.61E−08 |
| 613 | 9.29E−09 |
| 614 | 9.29E−09 |
| 615 | 1.20E−06 |
| 616 | 1.05E−07 |
| 617 | 3.19E−06 |
| 618 | 2.88E−07 |
| 619 | 2.76E−06 |
| 620 | 6.91E−08 |
| 621 | 3.23E−06 |
| 622 | 2.41E−07 |
| 623 | 1.49E−06 |
| 624 | 1.04E−06 |
| 625 | 1.14E−06 |
| 626 | 5.25E−07 |
| 627 | 2.17E−07 |
| 628 | 4.29E−07 |
| 629 | 2.76E−08 |
| 630 | 1.04E−08 |
| 631 | 4.87E−08 |
| 632 | 6.86E−07 |
| 633 | 2.08E−08 |
| 634 | 1.81E−07 |
| 635 | 7.78E−08 |
| 636 | 1.35E−07 |
| 637 | 1.95E−08 |
| 638 | 3.42E−08 |
| 639 | 6.37E−08 |
| 640 | 2.10E−08 |
| 641 | 4.49E−08 |
| 642 | 2.73E−08 |
| 643 | 4.43E−08 |
| 644 | 5.77E−07 |
| 645 | 9.73E−07 |
| 646 | 9.39E−08 |
| 647 | 1.19E−07 |
| 648 | 1.23E−06 |
| 649 | 2.74E−07 |
| 650 | 9.87E−07 |
| 651 | 1.14E−06 |
| 652 | 5.29E−09 |
| 653 | 1.41E−08 |
| 654 | 6.35E−09 |
| 655 | 2.35E−08 |
| 656 | 1.66E−08 |
| 657 | 2.04E−08 |
| 658 | 1.17E−08 |
| 659 | 8.79E−08 |
| 660 | 5.32E−08 |
| 661 | 4.75E−08 |
| 662 | 5.02E−07 |
| 663 | 3.47E−07 |
| 664 | 3.19E−07 |
| 665 | 2.93E−07 |
| 666 | 2.25E−08 |
| 667 | 1.18E−07 |
| 668 | 1.14E−07 |
| 669 | 1.48E−07 |
| 670 | 2.28E−07 |
| 671 | 4.00E−07 |
| 672 | 1.10E−07 |
| 673 | 2.24E−07 |
| 674 | 3.56E−07 |
| 675 | 2.98E−08 |
| 676 | 1.62E−06 |
| 677 | 1.12E−07 |
| 678 | 2.63E−07 |
| 679 | 4.89E−08 |
| 680 | 9.11E−08 |
| 681 | 7.15E−08 |
| 682 | 1.47E−07 |
| 683 | 9.91E−08 |
| 684 | 1.77E−07 |
| 685 | 3.67E−07 |
| 686 | 1.01E−08 |
| 687 | 1.08E−08 |
| 688 | 5.65E−09 |
| 689 | 5.28E−07 |
| 690 | 7.39E−07 |
| 691 | 2.65E−07 |
| 692 | 5.49E−07 |
| 693 | 5.54E−07 |
| 694 | 4.62E−08 |
| 695 | 5.89E−07 |
| 696 | 2.72E−08 |
| 697 | 2.80E−08 |
| 698 | 7.67E−08 |
| 699 | 1.52E−08 |
| 700 | 1.37E−08 |
| 701 | 8.89E−08 |
| 702 | 3.37E−07 |
| 703 | 5.59E−08 |
| 704 | 3.90E−08 |
| 705 | 1.96E−07 |
| 706 | 3.05E−08 |
| 707 | 1.46E−08 |
| 708 | 3.88E−07 |
| 709 | 4.84E−08 |
| 710 | 2.81E−08 |
| 711 | 6.11E−07 |
| 712 | 3.59E−08 |
| 713 | 1.63E−08 |
| 714 | 3.09E−07 |
| 715 | 4.64E−08 |
| 716 | 5.21E−08 |
| 717 | 8.19E−07 |
| 718 | 1.61E−08 |
| 719 | 1.13E−07 |
| 720 | 1.38E−08 |
| 721 | 3.65E−08 |
| 722 | 3.07E−08 |
| 723 | 8.36E−08 |
| 724 | 6.87E−09 |
| 725 | 5.32E−08 |
| 726 | 6.50E−09 |
| 727 | 2.83E−08 |
| 728 | 7.72E−08 |
| 729 | 6.49E−08 |
| 730 | 2.46E−07 |
| 731 | 2.13E−08 |
| 732 | 3.87E−08 |
| 733 | 1.64E−08 |
| 734 | 1.10E−07 |
| 735 | 3.76E−07 |
| 736 | 3.44E−07 |
| 737 | 1.37E−08 |
| 738 | 7.16E−09 |
| 739 | 2.16E−07 |
| 740 | 9.29E−09 |
| 741 | 6.97E−08 |
| 742 | 4.80E−09 |
| 743 | 9.45E−09 |
| 744 | 1.02E−07 |
| 745 | 5.28E−09 |
| 746 | 1.00E−08 |
| 747 | 5.18E−09 |
| 748 | 3.23E−08 |
| 749 | 8.32E−09 |
| 750 | 4.27E−08 |
| 751 | 3.90E−09 |
| 752 | 1.07E−08 |
| 753 | 3.41E−08 |
| 754 | 7.55E−09 |
| 755 | 1.11E−08 |
| 756 | 3.41E−08 |
| 757 | 4.18E−09 |
| 758 | 1.08E−08 |
| 759 | 2.54E−08 |
| 760 | 7.34E−09 |
| 761 | 1.78E−07 |
| 762 | 2.69E−07 |
| 763 | 3.01E−07 |
| 764 | 1.74E−08 |
| 765 | 7.04E−09 |
| 766 | 1.09E−07 |
| 767 | 8.52E−07 |
| 768 | 6.41E−07 |
| 769 | 3.11E−09 |
| 770 | 4.92E−08 |
| 771 | 1.27E−08 |
| 772 | 9.52E−10 |
| 773 | 2.23E−09 |
| 774 | 2.93E−09 |
| 775 | 3.43E−09 |
| 776 | 3.97E−09 |
| 777 | 3.61E−09 |
| 778 | 3.97E−09 |
| 779 | 5.23E−09 |
| 780 | 5.61E−09 |
| 781 | 5.95E−09 |
| 782 | 9.54E−09 |
| 783 | 5.06E−08 |
| 784 | 6.02E−09 |
| 785 | 7.63E−09 |
| 786 | 9.54E−09 |
| 787 | 1.53E−08 |
| 788 | 7.75E−09 |
| 789 | 7.75E−09 |
| 790 | 8.25E−09 |
| 791 | 8.31E−09 |
| 792 | 8.35E−09 |
| 793 | 9.84E−09 |
| 794 | 3.08E−08 |
| 795 | 1.13E−08 |
| 796 | 1.46E−07 |
| 797 | 6.74E−09 |
| 798 | 1.19E−08 |
| 799 | 2.13E−08 |
| 800 | 1.20E−08 |
| 801 | 4.41E−07 |
| 802 | 1.21E−08 |
| 803 | 1.28E−08 |
| 804 | 1.45E−08 |
| 805 | 1.66E−08 |
| 806 | 1.56E−08 |
| 807 | 2.96E−08 |
| 808 | 1.50E−08 |
| 809 | 1.63E−08 |
| 810 | 2.78E−08 |
| 811 | 1.83E−08 |
| 812 | 1.85E−08 |
| 813 | 2.43E−08 |
| 814 | 3.19E−08 |
| 815 | 2.50E−08 |
| 816 | 2.59E−08 |
| 817 | 2.67E−08 |
| 818 | 2.69E−08 |
| 819 | 4.49E−08 |
| 820 | 4.29E−08 |
| 821 | 3.19E−08 |
| 822 | 3.20E−08 |
| 823 | 1.10E−07 |
| 824 | 3.43E−08 |
| 825 | 3.99E−08 |
| 826 | 4.70E−08 |
| 827 | 4.97E−08 |
| 828 | 5.19E−08 |
| 829 | 5.46E−08 |
| 830 | 5.47E−08 |
| 831 | 5.52E−08 |
| 832 | 2.66E−07 |
| 833 | 1.51E−07 |
| 834 | 6.31E−08 |
| 835 | 1.07E−07 |
| 836 | 4.49E−08 |
| 837 | 2.85E−07 |
| 838 | 6.96E−08 |
| 839 | 6.99E−08 |
| 840 | 7.32E−08 |
| 841 | 7.39E−08 |
| 842 | 7.64E−08 |
| 843 | 7.92E−08 |
| 844 | 9.00E−08 |
| 845 | 9.32E−08 |
| 846 | 1.75E−07 |
| 847 | 1.41E−07 |
| 848 | 2.32E−06 |
| 849 | 2.79E−07 |
| 850 | 4.75E−07 |
| 851 | 6.22E−07 |
| 852 | 2.54E−07 |
| 853 | 1.47E−07 |
| 854 | 1.60E−07 |
| 855 | 1.68E−07 |
| 856 | 1.10E−07 |
| 857 | 1.22E−07 |
| 858 | 6.95E−08 |
| 859 | 5.07E−08 |
| 860 | 1.15E−08 |
| 861 | 1.77E−07 |
| 862 | 4.63E−08 |
| 863 | 2.88E−08 |
| 864 | 4.03E−07 |
| 865 | 6.42E−08 |
| 866 | 4.15E−08 |
| 867 | 8.14E−07 |
| 868 | 3.58E−07 |
| 869 | 1.23E−07 |
| 870 | 4.97E−07 |
| 871 | 6.94E−07 |
| 872 | 5.37E−07 |
| 873 | 1.39E−07 |
| 874 | 2.72E−07 |
| 875 | 1.80E−07 |
| 876 | 1.22E−07 |
| 877 | 1.90E−07 |
| 878 | |
| 879 | 1.43E−07 |
| 880 | 2.03E−07 |
| 881 | 1.69E−07 |
| 882 | 1.05E−07 |
| 883 | 1.08E−07 |
| 884 | 1.99E−07 |
| 885 | 1.08E−07 |
| 886 | 1.69E−07 |
| 887 | 5.82E−07 |
| 888 | 1.01E−06 |
| 889 | 5.00E−08 |
| 890 | 1.11E−07 |
| 891 | 1.11E−07 |
| 892 | 7.03E−09 |
| 893 | 6.36E−09 |
| 894 | 5.72E−08 |
| 895 | 4.50E−09 |
| 896 | 1.19E−07 |
| 897 | 1.13E−08 |
| 898 | 6.57E−07 |
| 899 | 5.36E−07 |
| 900 | 7.77E−08 |
| 901 | 2.22E−08 |
| 902 | 1.05E−07 |
| 903 | 6.57E−09 |
| 904 | 2.40E−08 |
| 905 | 1.21E−07 |
| 906 | 1.35E−08 |
| 907 | 1.33E−08 |
| 908 | 9.75E−09 |
| 909 | 2.34E−08 |
| 910 | 3.03E−08 |
| 911 | 1.92E−07 |
| 912 | 1.86E−08 |
| 913 | 4.00E−07 |
| 914 | 1.03E−07 |
| 915 | 4.84E−07 |
| 916 | 3.51E−08 |
| 917 | 3.19E−08 |
| 918 | 4.17E−08 |
| 919 | 8.25E−08 |
| 920 | 5.97E−09 |
| 921 | 2.78E−08 |
| 922 | 3.91E−08 |
| 923 | 8.19E−09 |
| 924 | 1.32E−08 |
| 925 | 2.04E−07 |
| 926 | 1.21E−08 |
| 927 | 1.03E−08 |
| 928 | 2.81E−07 |
| 929 | 7.97E−08 |
| 930 | 3.00E−08 |
| 931 | 1.34E−08 |
| 932 | 5.52E−08 |
| 933 | 7.65E−08 |
| 934 | 1.67E−08 |
| 934 | 1.67E−08 |
| 935 | 6.96E−09 |
| 936 | 1.87E−07 |
| 937 | 3.10E−08 |
| 938 | 8.49E−09 |
| 939 | 1.41E−07 |
| 940 | 2.66E−08 |
| 941 | 4.19E−08 |
| 942 | 4.92E−09 |
| 943 | 6.65E−09 |
| 944 | 2.91E−09 |
| 945 | 4.44E−08 |
| 946 | 3.62E−08 |
| 947 | 6.09E−08 |
| 948 | 2.12E−08 |
| 949 | 9.70E−09 |
| 950 | 6.43E−08 |
| 951 | 1.61E−07 |
| 952 | 2.18E−08 |
| 953 | 3.05E−08 |
| 954 | 4.81E−07 |
| 955 | 2.09E−07 |
| 956 | 9.01E−08 |
| 957 | 2.10E−07 |
| 958 | 4.34E−08 |
| 959 | 1.90E−09 |
| 960 | 1.41E−07 |
| 961 | 4.12E−09 |
| 962 | 2.70E−08 |
| 963 | 4.05E−08 |
| 964 | 7.12E−09 |
| 965 | 2.46E−08 |
| 966 | 1.12E−08 |
| 967 | 1.42E−08 |
| 968 | 5.51E−08 |
| 969 | 2.52E−07 |
| 970 | 1.49E−07 |
| 971 | 7.48E−08 |
| 972 | 1.58E−07 |
| 973 | 1.05E−08 |

1) Compound of formula (I) 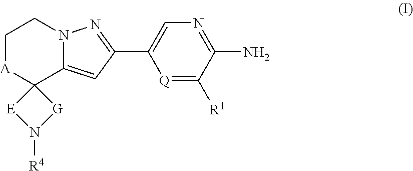
wherein
A is selected from a single bond, —CH₂— or —O—;
E is —(CH₂)ₙ— with n=1 or 2;
G is —(CH₂)— and with the condition, that the cyclic substructure comprising E-N-G is a 4 to 5 membered heterocycloalkyl;
Q is selected from N or C—R²;
R¹ is selected from
—NO₂; —CN;
C₁₋₄-alkyl optionally substituted by —OH or 1 to 3-F;
cyclopropyl;
4 to 6 membered heterocycloalkyl or heterocycloalkenyl, both with 1-2 nitrogen atoms, but always linked via a carbon atom to the phenyl group of compounds of formula (I) and both optionally substituted by C₁₋₄-alkyl;
phenyl optionally substituted by halogen, —CN, C₁₋₄-alkyl, C₁₋₄-haloalkyl, C₃₋₆-cycloalkyl, O—C₁₋₄-alkyl, O—C₃₋₆-cycloalky, C₁₋₃-alkinyl, —C(O)—R_{d}, SR_{d}, SOR_{d}, SO₂R_{d}, SONR_{d}, SON(H)R_{d}, —SO₂—NH₂, —SO₂—N(H)—R_{d} or —SO₂—N(R_{d})₂, wherein R_{d} is selected from methyl, ethyl, cyclopropyl, isopropyl and tert·butyl;
5 to 6 membered heteroaryl with 1, 2 or 3 heteroatoms selected from N, O or S and optionally substituted by 1 or 2 of the following substituents: halogen, —CN, C₁₋₄-alkyl, C₁₋₄-haloalkyl, C₃₋₆-cycloalkyl, O—C₁₋₄-alkyl, O—C₃₋₆-cycloalkyl, C₁₋₃-alkinyl, SR_{d}, SOR_{d}, SO₂R_{d}, SONR_{d} or SON(H)R_{d} wherein R_{d} is selected from methyl, ethyl, cyclopropyl, isopropyl and tert·butyl;
—O—CH₃; —O—CH(CH₃)₂; —O—CHF₂; —O—CF₃; 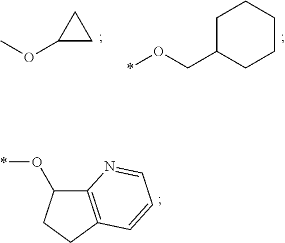
—O—CHRₐ-phenyl optionally substituted by one, two or three of the following substituents: halogen, —CN, C₁₋₄-alkyl optionally substituted by one or more —F, O—C₁₋₄-alkyl, C₁₋₆-alkinyl-OH or —SO₂—CH₃ and with Rₐ selected from —H, —F or —CH₃, —CF₃, —CH₂—CH₃, —CH(CH₃)₂ or -cyclopropyl;
—O—CHRₐ-6 membered heteroaryl optionally substituted one, two or three of the following substituents: halogen, —CN, C₁₋₄-alkyl optionally substituted by one or more —F or —SO₂—CH₃ and with Rₐ selected from —H, —F or —CH₃, —CF₃, —CH₂—CH₃, —CH(CH₃)₂ or -cyclopropyl;
—COORₐ with Rₐ selected from —H or —CH₃;
C(═O)-methyl, optionally substituted by 1 to 3-F;
—C(═O)—NR_{b}R_{c} wherein R_{b} is selected from —H, —CH₃ or —CH₂—CH₃ and wherein R_{c} is selected from —H, —CH₃, —CH₂—CH₃, cyclohexyl, phenyl or 5 to 6 membered heteroaryl, all optionally substituted by one or two halogen, —CN, —SO₂—CH₃ or C₁₋₄-alkyl, optionally substituted one or more times with halogen;
—S—CR_{f}R_{g}Rₕ, wherein R_{f} is selected from —H or —F, wherein R_{g} is selected from —H, —F or —CH₃ and wherein Rₕ is selected from H, F, —CH₃ or C₅₋₆-cycloalkyl or phenyl; 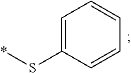 [SMILES: *Sc1ccccc1]
R² is selected from —H;
R⁴ is selected from
C₁₋₆-alkyl, C₃₋₆-cycloalkyl or C₁₋₃-alkyl-C₃₋₆-cycloalkyl, all optionally substituted one or independently more times by
—F, —Cl, —Br, —CN;
—OH, —ORᵢ wherein Rᵢ is selected from C₁₋₄-alkyl or C₃₋₆-Cycloalkyl, both optionally substituted one or more times by halogen,
—CF₃, —CHF₂, —CH₂F, or —CH₂—CF₃;
—NRⱼRₖ wherein Rⱼ and Rₖ are independently selected from —H and C₁-C₄-alkyl,
—CH₂—O—CH₃ or
C₃₋₄ cycloalkyl or 4-6 membered heterocycloalkyl, both optionally substituted by (═O), or optionally substituted one or more times by F or CH₃;
C₆₋₈-spiro-alkyl and C₅₋₇-bicyclo-alkyl
phenyl or 5 to 6 membered heteroaryl, both optionally substituted one or more times by
halogen, —CN,
—CF₃, —CHF₂, —CH₂F,
C₁₋₄-alkyl optionally substituted with one or more halogen;
C₃₋₆-cycloalkyl;
O—C₁₋₄-alkyl,
O—C₃₋₆-cycloalky,
C₁₋₃-alkinyl,
NRⱼRₖ wherein Rⱼ and Rₖ are independently selected from —H and C₁-C₄-alkyl
SR_{d}, SOR_{d}, SO₂R_{d}, SONR_{d} or SON(H)R_{d} wherein R_{d} is selected from methyl, ethyl, cyclopropyl, isopropyl and tert·butyl; or
phenyl is optionally substituted one or more times by —C(O)NRⱼRₖ wherein Rⱼ and Rₖ are independently selected from —H and C₁-C₄-alkyl or
5 to 6 membered heteroaryl is optionally substituted one or more times by —OH;
C(═O)—Rₗ in which Rₗ is selected from
C₁₋₄-alkyl, C₃₋₆-cycloalkyl or C₃₋₆-heterocycloalkyl, both optionally substituted one or more times by
—F, —Cl, —Br, —CN;
—OH, —ORᵢ with Rᵢ selected from C₁₋₄-alkyl or C₃₋₆Cycloalkyl), —CF₃, —CHF₂, —CH₂F;
—NRⱼRₖ wherein Rⱼ and Rₖ are independently selected from —H and C₁-C₄-alkyl
phenyl or 5 to 6 membered heteroaryl, both optionally substituted one or more times by
halogen, —CN,
—CF₃, —CHF₂, —CH₂F,
C₁₋₄-alkyl optionally substituted with one or more halogen;
C₃₋₆-cycloalkyl;
O—C₁₋₄-alkyl,
O—C₃₋₆-cycloalky,
C₁₋₃-alkinyl,
—NRⱼRₖ wherein Rⱼ and Rₖ are independently selected from —H and C₁-C₄-alkyl
SR_{d}, SOR_{d}, SO₂R_{d}, SONR_{d} or SON(H)R_{d} wherein R_{d} is selected from methyl, ethyl, cyclopropyl, isopropyl and tert·butyl;
phenyl or 5 to 6 membered heteroaryl, both optionally substituted one or more times by
halogen, —CN,
—CF₃, —CHF₂, —CH₂F,
C₁₋₄-alkyl optionally substituted with one or more halogen;
C₃₋₆-cycloalkyl;
O—C₁₋₄-alkyl,
O—C₃₋₆-cycloalky,
C₁₋₃-alkinyl,
—NRⱼRₖ wherein Rⱼ and Rₖ are independently selected from —H and C₁-C₄-alkyl
SR_{d}, SOR_{d}, SO₂R_{d}, SONR_{d} or SON(H)R_{d} wherein R_{d} is selected from methyl, ethyl, cyclopropyl, isopropyl and tert·butyl;
C(═O)—NRₒRₚ wherein Rₒ and Rₚ are independently selected from
H
C₁₋₄-alkyl, C₃₋₆-cycloalkyl or C₁₋₃-alkyl-C₃₋₆-cycloalkyl, both optionally substituted one or more times by
—F, —Cl, —Br, —CN;
—OH, —ORᵢ with Rᵢ selected from C₁₋₄-alkyl or C₃₋₆-cycloalkyl, —CF₃, —CHF₂, —CH₂F;
—NRⱼRₖ wherein Rⱼ and Rₖ are independently selected from —H and C₁-C₄-alkyl
phenyl or 5 to 6 membered heteroaryl, both optionally substituted one or more times by
halogen, —CN,
—CF₃, —CHF₂, —CH₂F,
C₁₋₄-alkyl optionally substituted with one or more halogen;
C₃₋₆-cycloalkyl;
O—C₁₋₄-alkyl,
O—C₃₋₆-cycloalky,
C₁₋₃-alkinyl,
—NRⱼRₖ wherein Rⱼ and Rₖ are independently selected from —H and C₁-C₄-alkyl
SR_{d}, SOR_{d}, SO₂R_{d}, SONR_{d} or SON(H)R_{d} wherein R_{d} is selected from methyl, ethyl, cyclopropyl, isopropyl and tert·butyl;
phenyl or 5 to 6 membered heteroaryl, both optionally substituted one or more times by
halogen, —CN,
—CF₃, —CHF₂, —CH₂F,
C₁₋₄-alkyl optionally substituted with one or more halogen;
C₃₋₆-cycloalkyl;
O—C₁₋₄-alkyl,
O—C₃₋₆-cycloalky,
C₁₋₃-alkinyl,
—NRⱼRₖ wherein Rⱼ and Rₖ are independently selected from —H and C₁-C₄-alkyl
SR_{d}, SOR_{d}, SO₂R_{d}, SONR_{d} or SON(H)R_{d} wherein R_{d} is selected from methyl, ethyl, cyclopropyl, isopropyl and tert·butyl;
or a stereoisomer, a tautomer, an N-oxide, a hydrate, a solvate, or a salt thereof, or a mixture of same.

2) Compound of formula (Ia) according to claim 1 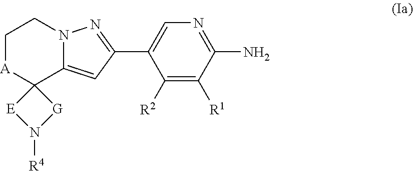
wherein
A is selected from a single bond, —CH₂— or —O—;
E is —(CH₂)ₙ— with n=1 or 2;
G is —(CH₂)— and with the condition, that the cyclic substructure comprising E-N-G is a 4 to 5 membered heterocycloalkyl;
R¹ is selected from
—NO₂; —CN;
C₁₋₄-alkyl optionally substituted by —OH or 1 to 3-F;
cyclopropyl;
4 to 6 membered heterocycloalkyl or heterocycloalkenyl, both with 1-2 nitrogen atoms, but always linked via a carbon atom to the phenyl group of compounds of formula (I) and both optionally substituted by C₁₋₄-alkyl;
phenyl optionally substituted by halogen, —CN, C₁₋₄-alkyl, C₁₋₄-haloalkyl, C₃₋₆-cycloalkyl, O—C₁₋₄-alkyl, O—C₃₋₆-cycloalky, C₁₋₃-alkinyl, —C(O)—R_{d}, SR_{d}, SOR_{d}, SO₂R_{d}, SONR_{d}, SON(H)R_{d}, —SO₂—NH₂, —SO₂—N(H)—R_{d} or —SO₂—N(R_{d})₂, wherein R_{d} is selected from methyl, ethyl, cyclopropyl, isopropyl and tert·butyl;
5 to 6 membered heteroaryl with 1 or 2 heteroatoms selected from N or S and optionally substituted by halogen, —CN, C₁₋₄-alkyl, C₁₋₄-haloalkyl, C₃₋₆-cycloalkyl, O—C₁₋₄-alkyl, O—C₃₋₆-cycloalkyl, C₁₋₃-alkinyl, SR_{d}, SOR_{d}, SO₂R_{d}, SONR_{d} or SON(H)R_{d} wherein R_{d} is selected from methyl, ethyl, cyclopropyl, isopropyl and tert·butyl;
—O—CH₃; —O—CH(CH₃)₂; —O—CHF₂; —O—CF₃; 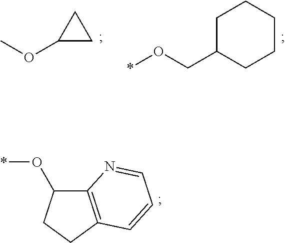
—O—CHRₐ-phenyl optionally substituted by one or two of the following substituents: halogen, —CN, C₁₋₄-alkyl optionally substituted by one or more —F or —SO₂—CH₃ and with Rₐ selected from —H, —F or —CH₃, —CF₃, —CH₂—CH₃, —CH(CH₃)₂ or -cyclopropyl;
—O—CHRₐ-6 membered heteroaryl optionally substituted one or two of the following substituents: halogen, —CN, C₁₋₄-alkyl optionally substituted by one or more —F or —SO₂—CH₃ and with Rₐ selected from —H, —F or —CH₃, —CF₃, —CH₂—CH₃, —CH(CH₃)₂ or -cyclopropyl;
—COORₐ with Rₐ selected from —H or —CH₃;
C(═O)-methyl, optionally substituted by 1 to 3-F;
—C(═O)—NR_{b}R_{c} wherein R_{b} is selected from —H, —CH₃ or —CH₂—CH₃ and wherein R_{c} is selected from —H, —CH₃, —CH₂—CH₃, cyclohexyl, phenyl or 5 to 6 membered heteroaryl, all optionally substituted by one or two halogen, —CN, —SO₂—CH₃ or C₁₋₄-alkyl, optionally substituted one or more times with halogen;
—S—CR_{f}R_{g}Rₕ, wherein R_{f} is selected from —H or —F, wherein R_{g} is selected from —H, —F or —CH₃ and wherein Rₕ is selected from H, F, —CH₃ or C₅₋₆-cycloalkyl or phenyl; 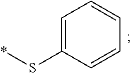 [SMILES: *Sc1ccccc1]
R² is selected from —H, or
R⁴ is selected from
C₁₋₅-alkyl or C₃₋₆-cycloalkyl, both optionally substituted one or independently more times by
—F, —Cl, —Br, —CN;
—OH, —ORᵢ wherein Rᵢ is selected from C₁₋₄-alkyl or C₃₋₆Cycloalkyl),
—CF₃, —CHF₂, —CH₂F, or —CH₂—CF₃;
—NRⱼRₖ wherein Rⱼ and Rₖ are independently selected from —H and C₁-C₄-alkyl,
—CH₂—O—CH₃ or
C₃₋₄ cycloalkyl or 4 membered heterocycloalkyl, both optionally substituted by —F or —CH₃;
phenyl or 5 to 6 membered heteroaryl, both optionally substituted one or more times by
halogen, —CN,
—CF₃, —CHF₂, —CH₂F,
C₁₋₄-alkyl optionally substituted with one or more halogen;
C₃₋₆-cycloalkyl;
O—C₁₋₄-alkyl,
O—C₃₋₆-cycloalky,
C₁₋₃-alkinyl,
—NRⱼRₖ wherein Rⱼ and Rₖ are independently selected from —H and C₁-C₄-alkyl
SR_{d}, SOR_{d}, SO₂R_{d}, SONR_{d} or SON(H)R_{d} wherein R_{d} is selected from methyl, ethyl, cyclopropyl, isopropyl and tert·butyl;
C(═O)—Rₗ in which Rₗ is selected from
C₁₋₄-alkyl, C₃₋₆-cycloalkyl or C₃₋₆-heterocycloalkyl, both optionally substituted one or more times by
—F, —Cl, —Br, —CN;
—OH, —ORᵢ with Rᵢ selected from C₁₋₄-alkyl or C₃₋₆Cycloalkyl), —CF₃, —CHF₂, —CH₂F;
—NRⱼRₖ wherein Rⱼ and Rₖ are independently selected from —H and C₁-C₄-alkyl
phenyl or 5 to 6 membered heteroaryl, both optionally substituted one or more times by
halogen, —CN,
—CF₃, —CHF₂, —CH₂F,
C₁₋₄-alkyl optionally substituted with one or more halogen;
C₃₋₆-cycloalkyl;
O—C₁₋₄-alkyl,
O—C₃₋₆-cycloalky,
C₁₋₃-alkinyl,
—NRⱼRₖ wherein Rⱼ and Rₖ are independently selected from —H and C₁-C₄-alkyl
SR_{d}, SOR_{d}, SO₂R_{d}, SONR_{d} or SON(H)R_{d} wherein R_{d} is selected from methyl, ethyl, cyclopropyl, isopropyl and tert·butyl;
phenyl or 5 to 6 membered heteroaryl, both optionally substituted one or more times by
halogen, —CN,
—CF₃, —CHF₂, —CH₂F,
C₁₋₄-alkyl optionally substituted with one or more halogen;
C₃₋₆-cycloalkyl;
O—C₁₋₄-alkyl,
O—C₃₋₆-cycloalky,
C₁₋₃-alkinyl,
—NRⱼRₖ wherein Rⱼ and Rₖ are independently selected from —H and C₁-C₄-alkyl
SR_{d}, SOR_{d}, SO₂R_{d}, SONR_{d} or SON(H)R_{d} wherein R_{d} is selected from methyl, ethyl, cyclopropyl, isopropyl and tert·butyl;
C(═O)—NRₒRₚ wherein Rₒ and Rₚ are independently selected from
H
C₁₋₄-alkyl or C₃₋₆-cycloalkyl, both optionally substituted one or more times by
—F, —Cl, —Br, —CN;
—OH, —ORᵢ with Rᵢ selected from C₁₋₄-alkyl or C₃₋₆-cycloalkyl, —CF₃, —CHF₂, —CH₂F;
—NRⱼRₖ wherein Rⱼ and Rₖ are independently selected from —H and C₁-C₄-alkyl
phenyl or 5 to 6 membered heteroaryl, both optionally substituted one or more times by
halogen, —CN,
—CF₃, —CHF₂, —CH₂F,
C₁₋₄-alkyl optionally substituted with one or more halogen;
C₃₋₆-cycloalkyl;
O—C₁₋₄-alkyl,
O—C₃₋₆-cycloalky,
C₁₋₃-alkinyl,
—NRⱼRₖ wherein Rⱼ and Rₖ are independently selected from —H and C₁-C₄-alkyl
SR_{d}, SOR_{d}, SO₂R_{d}, SONR_{d} or SON(H)R_{d} wherein R_{d} is selected from methyl, ethyl, cyclopropyl, isopropyl and tert·butyl;
phenyl or 5 to 6 membered heteroaryl, both optionally substituted one or more times by
halogen, —CN,
—CF₃, —CHF₂, —CH₂F,
C₁₋₄-alkyl optionally substituted with one or more halogen;
C₃₋₆-cycloalkyl;
O—C₁₋₄-alkyl,
O—C₃₋₆-cycloalky,
C₁₋₃-alkinyl,
—NRⱼRₖ wherein Rⱼ and Rₖ are independently selected from —H and C₁-C₄-alkyl
SR_{d}, SOR_{d}, SO₂R_{d}, SONR_{d} or SON(H)R_{d} wherein R_{d} is selected from methyl, ethyl, cyclopropyl, isopropyl and tert·butyl;
or a stereoisomer, a tautomer, an N-oxide, a hydrate, a solvate, or a salt thereof, or a mixture of same.

3) Compounds of formula (I) according to claim 1, wherein A is selected from a single bond or —O— or a stereoisomer, a tautomer, an N-oxide, a hydrate, a solvate, or a salt thereof, or a mixture of same.

4) Compounds of formula (I) according to claim 1, wherein E is —CH₂—CH₂— or a stereoisomer, a tautomer, an N-oxide, a hydrate, a solvate, or a salt thereof, or a mixture of same.

5) Compounds of formula (I) according to claim 1, wherein Q is C—R² or a stereoisomer, a tautomer, an N-oxide, a hydrate, a solvate, or a salt thereof, or a mixture of same.

6) Compounds of formula (I) according to claim 1, wherein Q is N or a stereoisomer, a tautomer, an N-oxide, a hydrate, a solvate, or a salt thereof, or a mixture of same.

7) Compounds of formula (I) according to claim 1, wherein R₄ is 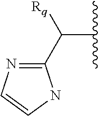
and R_{q} is selected from H, —CH₃, —CH₂—CH₃, —CH(CH₃)₂, —CH₂—O—CH₃, —CF₃, —CHF₂, —CH₂F, —CH₂—CF₃, cyclopropyl or cyclobutyl or a stereoisomer, a tautomer, an N-oxide, a hydrate, a solvate, or a salt thereof, or a mixture of same.

8) Compounds of formula (I) according to claim 1, wherein R₄ is selected from C(═O)—NRₒRₚ wherein Rₒ and Rₚ are independently selected from
H
C₁₋₄-alkyl or C₃₋₆-cycloalkyl, both optionally substituted one or more times by
—F, —Cl, —Br, —CN;
—OH, —ORᵢ with Rᵢ selected from C₁₋₄-alkyl or C₃₋₆-cycloalkyl, —CF₃, —CHF₂, —CH₂F;
—NRⱼRₖ wherein Rⱼ and Rₖ are independently selected from —H and C₁-C₄-alkyl
phenyl or 5 to 6 membered heteroaryl, both optionally substituted one or more times by
halogen, —CN,
—CF₃, —CHF₂, —CH₂F,
C₁₋₄-alkyl optionally substituted with one or more halogen;
C₃₋₆-cycloalkyl;
O—C₁₋₄-alkyl,
O—C₃₋₆-cycloalky,
C₁₋₃-alkinyl,
—NRⱼRₖ wherein Rⱼ and Rₖ are independently selected from —H and C₁-C₄-alkyl
SR_{d}, SOR_{d}, SO₂R_{d}, SONR_{d} or SON(H)R_{d} wherein R_{d} is selected from methyl, ethyl, cyclopropyl, isopropyl and tert·butyl;
or a stereoisomer, a tautomer, an N-oxide, a hydrate, a solvate, or a salt thereof, or a mixture of same.

9) Compound according to claim 1, which is selected from:
(rac)-2′-[6-amino-5-(trifluoromethyl)pyridin-3-yl]-N-[1-(2-chlorophenyl)cyclobutyl]-5′,6′-dihydrospiro[pyrrolidine-3,4′-pyrrolo[1,2-b]pyrazole]-1-carboxamide
(rac)-2-[6-amino-5-(trifluoromethyl)pyridin-3-yl]-N-[2-(2-fluorophenyl)propan-2-yl]-6,7-dihydrospiro[pyrazolo[5,1-c][1,4]oxazine-4,3′-pyrrolidine]-1′-carboxamide
(rac)-2-[6-amino-5-(trifluoromethyl)pyridin-3-yl]-N-(1-phenylcyclobutyl)-6,7-dihydrospiro[pyrazolo[5,1-c][1,4]oxazine-4,3′-pyrrolidine]-1′-carboxamide
(rac)-2′-[6-amino-5-(trifluoromethyl)pyridin-3-yl]-N-[2-(4-fluorophenyl)propan-2-yl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[2-(2-chlorophenyl)propan-2-yl]-5′,6′-dihydrospiro[pyrrolidine-3,4′-pyrrolo[1,2-b]pyrazole]-1-carboxamide
(rac)-2-[6-amino-5-(trifluoromethyl)pyridin-3-yl]-N-[(1R)-1-(2-chloro-4-cyanophenyl)ethyl]-6,7-dihydrospiro[pyrazolo[5,1-c][1,4]oxazine-4,3′-pyrrolidine]-1′-carboxamide
(rac)-2′-[6-amino-5-(trifluoromethyl)pyridin-3-yl]-N-[(1R)-1-(2-chloro-4-cyanophenyl)ethyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[(1S)-1-(3-chloropyridin-4-yl)ethyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[2-(3-fluorophenyl)propan-2-yl]-5′,6′-dihydrospiro[pyrrolidine-3,4′-pyrrolo[1,2-b]pyrazole]-1-carboxamide
(rac)-2-[6-amino-5-(trifluoromethyl)pyridin-3-yl]-N-[1-(pyridin-4-yl)cyclobutyl]-6,7-dihydrospiro[pyrazolo[5,1-c][1,4]oxazine-4,3′-pyrrolidine]-1′-carboxamide
(rac)-2′-[6-amino-5-(trifluoromethyl)pyridin-3-yl]-N-[2-(3-chloropyridin-4-yl)propan-2-yl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-(1-phenylcyclobutyl)-5′,6′-dihydrospiro[pyrrolidine-3,4′-pyrrolo[1,2-b]pyrazole]-1-carboxamide
(rac)-2-[6-amino-5-(trifluoromethyl)pyridin-3-yl]-N-[2-(pyridin-2-yl)propan-2-yl]-6,7-dihydrospiro[pyrazolo[5,1-c][1,4]oxazine-4,3′-pyrrolidine]-1′-carboxamide
(rac)-2′-(5-acetyl-6-aminopyridin-3-yl)-N-(1-phenylcyclobutyl)-5′,6′-dihydrospiro[pyrrolidine-3,4′-pyrrolo[1,2-b]pyrazole]-1-carboxamide
(rac)-2-(6-amino-5-cyanopyridin-3-yl)-N-[1-(2-chlorophenyl)cyclobutyl]-6,7-dihydrospiro[pyrazolo[5,1-c][1,4]oxazine-4,3′-pyrrolidine]-1′-carboxamide
(rac)-2′-(6-amino-5-cyanopyridin-3-yl)-N-[1-(2-chlorophenyl)cyclobutyl]-5′,6′-dihydrospiro[pyrrolidine-3,4′-pyrrolo[1,2-b]pyrazole]-1-carboxamide
(rac)-2-[6-amino-5-(trifluoromethyl)pyridin-3-yl]-N-[2-(3-chloropyridin-4-yl)propan-2-yl]-6,7-dihydrospiro[pyrazolo[5,1-c][1,4]oxazine-4,3′-pyrrolidine]-1′-carboxamide
(rac)-2-(6-amino-5-cyanopyridin-3-yl)-N-[1-(pyridin-4-yl)cyclobutyl]-6,7-dihydrospiro[pyrazolo[5,1-c][1,4]oxazine-4,3′-pyrrolidine]-1′-carboxamide
(rac)-2-[6-amino-5-(trifluoromethyl)pyridin-3-yl]-N-[1-(2-fluorophenyl)cyclobutyl]-6,7-dihydrospiro[pyrazolo[5,1-c][1,4]oxazine-4,3′-pyrrolidine]-1′-carboxamide
(rac)-2′-(6-amino-5-cyanopyridin-3-yl)-N-(1-phenylcyclobutyl)-5′,6′-dihydrospiro[pyrrolidine-3,4′-pyrrolo[1,2-b]pyrazole]-1-carboxamide
(rac)-2-(6-amino-5-cyanopyridin-3-yl)-N-[1-(4-fluorophenyl)cyclobutyl]-6,7-dihydrospiro[pyrazolo[5,1-c][1,4]oxazine-4,3′-pyrrolidine]-1′-carboxamide
(rac)-2′-(6-amino-5-cyanopyridin-3-yl)-N-[1-(4-fluorophenyl)cyclobutyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[1-(2-fluorophenyl)cyclobutyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[1-(3-fluorophenyl)cyclobutyl]-5′,6′-dihydrospiro[pyrrolidine-3,4′-pyrrolo[1,2-b]pyrazole]-1-carboxamide
(rac)-2′-(6-amino-5-cyanopyridin-3-yl)-N-[2-(3-chloropyridin-4-yl)propan-2-yl]-5′,6′-dihydrospiro[pyrrolidine-3,4′-pyrrolo[1,2-b]pyrazole]-1-carboxamide
(rac)-2′-(6-amino-5-cyanopyridin-3-yl)-N-[1-(pyridin-4-yl)cyclobutyl]-5′,6′-dihydrospiro[pyrrolidine-3,4′-pyrrolo[1,2-b]pyrazole]-1-carboxamide
(rac)-2-(6-amino-5-cyanopyridin-3-yl)-N-(1-phenylcyclobutyl)-6,7-dihydrospiro[pyrazolo[5,1-c][1,4]oxazine-4,3′-pyrrolidine]-1′-carboxamide
(rac)-2-(6-amino-5-cyanopyridin-3-yl)-N-[2-(4-fluorophenyl)propan-2-yl]-6,7-dihydrospiro[pyrazolo[5,1-c][1,4]oxazine-4,3′-pyrrolidine]-1′-carboxamide
(rac)-2′-[6-amino-5-(trifluoromethyl)pyridin-3-yl]-N-[1-(pyridin-4-yl)cyclobutyl]-5′,6′-dihydrospiro[pyrrolidine-3,4′-pyrrolo[1,2-b]pyrazole]-1-carboxamide
(rac)-2-[6-amino-5-(trifluoromethyl)pyridin-3-yl]-N-[1-(3-fluorophenyl)cyclobutyl]-6,7-dihydrospiro[pyrazolo[5,1-c][1,4]oxazine-4,3′-pyrrolidine]-1′-carboxamide
2′-[6-amino-5-(trifluoromethyl)pyridin-3-yl]-N-{(1R)-1-[3-(trifluoromethyl)phenyl]ethyl}-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[2-(3-fluoropyridin-2-yl)propan-2-yl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[2-(pyridin-4-yl)propan-2-yl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[2-(2-fluorophenyl)propan-2-yl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1R)-1-(3-fluorophenyl)ethyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[2-(pyridin-3-yl)propan-2-yl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[1-(pyridin-3-yl)cyclobutyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1R)-1-(4-fluorophenyl)ethyl]-5′,6′-dihydrospiro[pyrrolidine-3,4′-pyrrolo[1,2-b]pyrazole]-1-carboxamide
2′-[6-amino-5-(trifluoromethyl) pyridin-3-yl]-N-[(1R)-1-phenylpropyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-{(1S)-1-[3-(trifluoromethyl)phenyl]ethyl}-5′,6′-dihydrospiro[pyrrolidine-3,4′-pyrrolo[1,2-b]pyrazole]-1-carboxamide
2′-[6-amino-5-(trifluoromethyl) pyridin-3-yl]-N-[(1R)-1-phenylethyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[2-(pyridin-2-yl)propan-2-yl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1R)-1-(2-cyanophenyl)ethyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[1-(1,3,5-trimethyl-1H-pyrazol-4-yl)ethyl]-5′,6′-dihydrospiro[pyrrolidine-3,4′-pyrrolo[1,2-b]pyrazole]-1-carboxamide (diastereomeric mixture)
2′-[6-amino-5-(trifluoromethyl)pyridin-3-yl]-N-[(1R)-2,3-dihydro-1H-inden-1-yl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1R)-1-(2-cyanopyridin-4-yl)ethyl]-5′,6′-dihydrospiro[pyrrolidine-3,4′-pyrrolo[1,2-b]pyrazole]-1-carboxamide
2′-[6-amino-5-(trifluoromethyl) pyridin-3-yl]-N-[1-(1-ethyl-3,5-dimethyl-1H-pyrazol-4-yl)ethyl]-5′,6′-dihydrospiro[pyrrolidine-3,4′-pyrrolo[1,2-b]pyrazole]-1-carboxamide (diastereometric mixture)
(rac)-2′-[6-amino-5-(trifluoromethyl)pyridin-3-yl]-N-[1-(pyridin-2-yl)cyclobutyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[1-(1,3,4-trimethyl-1H-pyrazol-5-yl)ethyl]-5′,6′-dihydrospiro[pyrrolidine-3,4′-pyrrolo[1,2-b]pyrazole]-1-carboxamide (diastereometric mixture)
2′-[6-amino-5-(trifluoromethyl)pyridin-3-yl]-N-[(1R)-1-(4-cyanopyridin-2-yl)ethyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1R)-1-(pyridin-3-yl)ethyl]-5′,6′-dihydrospiro[pyrrolidine-3,4′-pyrrolo[1,2-b]pyrazole]-1-carboxamide
(rac)-2′-[6-amino-5-(trifluoromethyl) pyridin-3-yl]-N-[2-(1-methyl-1H-1,2,3-triazol-4-yl)propan-2-yl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-{[3-(trifluoromethyl)phenyl]methyl}-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[1-(1-phenyl-1H-pyrazol-4-yl)ethyl]-5′,6′-dihydrospiro[pyrrolidine-3,4′-pyrrolo[1,2-b]pyrazole]-1-carboxamide (diastereometric mixture)
2′-[6-amino-5-(trifluoromethyl)pyridin-3-yl]-N-[(3S)-2,3-dihydro-1-benzofuran-3-yl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1R)-1-(6-cyanopyridin-3-yl)ethyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-{(1S)-1-[1-methyl-3-(trifluoromethyl)-1H-pyrazol-4-yl]ethyl}-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1R)-1-(pyridin-2-yl)ethyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-benzyl-5′,6′-dihydrospiro[pyrrolidine-3,4′-pyrrolo[1,2-b]pyrazole]-1-carboxamide
2′-[6-amino-5-(trifluoromethyl) pyridin-3-yl]-N-[(1S)-1-phenylethyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1S)-1-(1,2-oxazol-3-yl)ethyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1S)-1-(1,5-dimethyl-1H-pyrazol-4-yl)ethyl]-5′,6′-dihydrospiro[pyrrolidine-3,4′-pyrrolo[1,2-b]pyrazole]-1-carboxamide (diastereomeric mixture)
(rac)-2′-[6-amino-5-(trifluoromethyl)pyridin-3-yl]-N-[2-(oxan-4-yl)propan-2-yl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1R)-1-(pyridin-4-yl)ethyl]-5′,6′-dihydrospiro[pyrrolidine-3,4′-pyrrolo[1,2-b]pyrazole]-1-carboxamide
2′-[6-amino-5-(trifluoromethyl) pyridin-3-yl]-N-[(1S)-1-(1-ethyl-5-methyl-1H-pyrazol-4-yl)ethyl]-5′,6′-dihydrospiro[pyrrolidine-3,4′-pyrrolo[1,2-b]pyrazole]-1-carboxamide (diastereomeric mixture)
(rac)-2′-[6-amino-5-(trifluoromethyl)pyridin-3-yl]-N-[(pyridin-3-yl)methyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1S)-2-methoxy-1-phenylethyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-{[3-(trifluoromethyl)bicyclo[1.1.1]pentan-1-yl]methyl}-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[(pyridin-4-yl)methyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1R)-1-(3-cyanopyridin-2-yl)ethyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1S)-1-(1-methyl-1H-benzimidazol-2-yl)ethyl]-5′,6′-dihydrospiro[pyrrolidine-3,4′-pyrrolo[1,2-b]pyrazole]-1-carboxamide (diastereomeric mixture)
2′-[6-amino-5-(trifluoromethyl)pyridin-3-yl]-N-[1-(4-fluorophenyl)-2-methoxyethyl]-5′,6′-dihydrospiro[pyrrolidine-3,4′-pyrrolo[1,2-b]pyrazole]-1-carboxamide (mixture of isomers)
(rac)-2′-[6-amino-5-(trifluoromethyl)pyridin-3-yl]-N-[(pyridin-2-yl)methyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1S)-1-(1-ethyl-1H-imidazol-2-yl)ethyl]-5′,6′-dihydrospiro[pyrrolidine-3,4′-pyrrolo[1,2-b]pyrazole]-1-carboxamide (diastereomeric mixture)
2′-[6-amino-5-(trifluoromethyl)pyridin-3-yl]-N-[(1S)-1-(1-methyl-1H-imidazol-2-yl)ethyl]-5′,6′-dihydrospiro[pyrrolidine-3,4′-pyrrolo[1,2-b]pyrazole]-1-carboxamide (diastereomeric mixture)
(rac)-2′-[6-amino-5-(trifluoromethyl)pyridin-3-yl]-N-[(pyrazin-2-yl)methyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1R)-2-methoxy-1-(pyridin-3-yl)ethyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[(pyridazin-4-yl)methyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[(1-methylpiperidin-4-yl)methyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[3-(methylsulfamoyl)pyridin-2-yl]-5′,6′-dihydrospiro[pyrrolidine-3,4′-pyrrolo[1,2-b]pyrazole]-1-carboxamide
(rac)-2′-[6-amino-5-(trifluoromethyl) pyridin-3-yl]-N-[(1-methyl-2-oxabicyclo[2.1.1]hexan-4-yl)methyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[(1-methyl-2-oxopiperidin-4-yl)methyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-(2-cyclopropylpropan-2-yl)-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1R)-1-(4-cyanophenyl)ethyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[1-methyl-2,3-dihydro-1H-inden-1-yl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[cyclopropyl(phenyl)methyl]-5′,6′-dihydrospiro[pyrrolidine-3,4′-pyrrolo[1,2-b]pyrazole]-1-carboxamide (mixture of isomers)
2′-[6-amino-5-(trifluoromethyl)pyridin-3-yl]-N-[−2-methyl-1-phenylpropyl]-5′,6′-dihydrospiro[pyrrolidine-3,4′-pyrrolo[1,2-b]pyrazole]-1-carboxamide (mixture of isomers)
2′-[6-amino-5-(trifluoromethyl) pyridin-3-yl]-N-[(1S)-1-(pyridin-4-yl)ethyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[2,2,2-trifluoro-1-phenylethyl]-5′,6′-dihydrospiro[pyrrolidine-3,4′-pyrrolo[1,2-b]pyrazole]-1-carboxamide (mixture of isomers)
2′-[6-amino-5-(trifluoromethyl) pyridin-3-yl]-N-[1-(pyrazin-2-yl)ethyl]-5′,6′-dihydrospiro[pyrrolidine-3,4′-pyrrolo[1,2-b]pyrazole]-1-carboxamide (mixture of isomers)
2′-[6-amino-5-(trifluoromethyl)pyridin-3-yl]-N-[(1R)-2-methoxy-1-phenylethyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[(pyridazin-3-yl)methyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[1-(2-cyanophenyl)cyclobutyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1S)-2,3-dihydro-1H-inden-1-yl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1S)-1-(4-fluorophenyl)ethyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1R)-1-(2-cyanophenyl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(1R)-1-(6-cyanopyridin-3-yl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[1-(pyridin-2-yl)cyclobutyl]-5′,6′-dihydrospiro[azetidin-3,4′-pyrrolo[1,2-b]pyrazole]-1-carboxamide
2′-[6-amino-5-(trifluoromethyl)pyridin-3-yl]-N-[(pyridin-4-yl)methyl]-5′,6′-dihydrospiro[azetidine-3,4′-pyrrolo[1,2-b]pyrazole]-1-carboxamide
(3′RS)-2-[6-amino-5-(trifluoromethyl)pyridin-3-yl]-N-[(1R)-1-(4-cyanophenyl)ethyl]-6,7-dihydrospiro[pyrazolo[5,1-c][1,4]oxazine-4,3′-pyrrolidine]-1′-carboxamide
2′-[6-amino-5-(trifluoromethyl)pyridin-3-yl]-N-[(1R)-1-(3-cyanopyridin-2-yl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(1R)-1-(4-cyanophenyl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(1R)-1-(2-cyanopyridin-4-yl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(1R)-1-(2-chloro-4-cyanophenyl)ethyl]-5′,6′-dihydrospiro[azetidine-3,4′-pyrrolo[1,2-b]pyrazole]-1-carboxamide
2′-[6-amino-5-(trifluoromethyl) pyridin-3-yl]-N-[2-(2-fluorophenyl) propan-2-yl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[1-(1-ethyl-3,5-dimethyl-1H-pyrazol-4-yl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[2-(pyridin-2-yl)propan-2-yl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[1-(1-methyl-1H-pyrazol-4-yl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[1-(1,3,4-trimethyl-1H-pyrazol-5-yl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-{1-[3-(difluoromethyl)-1-methyl-1H-pyrazol-4-yl]ethyl}-5′,6′-dihydrospiro[azetidine-3,4′-pyrrolo[1,2-b]pyrazole]-1-carboxamide
(rac)-2-[6-amino-5-(trifluoromethyl)pyridin-3-yl]-N-[2-(pyridin-4-yl)propan-2-yl]-6,7-dihydrospiro[pyrazolo[5,1-c][1,4]oxazine-4,3′-pyrrolidine]-1′-carboxamide
(rac)-2-[6-amino-5-(trifluoromethyl)pyridin-3-yl]-N-[1-(pyridin-3-yl)cyclobutyl]-6,7-dihydrospiro[pyrazolo[5,1-c][1,4]oxazine-4,3′-pyrrolidine]-1′-carboxamide
2′-[6-amino-5-(trifluoromethyl)pyridin-3-yl]-N-[1-(1-ethyl-5-methyl-1H-pyrazol-4-yl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[1-(1,5-dimethyl-1H-pyrazol-4-yl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[1-(1,3,5-trimethyl-1H-pyrazol-4-yl)ethyl]-5′,6′-dihydrospiro[azetidine-3,4′-pyrrolo[1,2-b]pyrazole]-1-carboxamide
2′-[6-amino-5-(trifluoromethyl) pyridin-3-yl]-N-[(1R)-1-(pyridin-3-yl)ethyl]-5′,6′-dihydrospiro[azetidine-3,4′-pyrrolo[1,2-b]pyrazole]-1-carboxamide
2-[6-amino-5-(trifluoromethyl)pyridin-3-yl]-N-[(1R)-1-(2-cyanopyridin-4-yl)ethyl]-6,7-dihydrospiro[pyrazolo[5,1-c][1,4]oxazine-4,3′-pyrrolidine]-1′-carboxamide
2-[6-amino-5-(trifluoromethyl)pyridin-3-yl]-N-[(1R)-1-(3-cyanopyridin-2-yl)ethyl]-6,7-dihydrospiro[pyrazolo[5,1-c][1,4]oxazine-4,3′-pyrrolidine]-1′-carboxamide
2-[6-amino-5-(trifluoromethyl)pyridin-3-yl]-N-[(1R)-1-(6-cyanopyridin-3-yl)ethyl]-6,7-dihydrospiro[pyrazolo[5,1-c][1,4]oxazine-4,3′-pyrrolidine]-1′-carboxamide
2′-[6-amino-5-(trifluoromethyl)pyridin-3-yl]-N-[(pyridin-2-yl)methyl]-5′,6′-dihydrospiro[azetidine-3,4′-pyrrolo[1,2-b]pyrazole]-1-carboxamide
(rac)-2-[6-amino-5-(trifluoromethyl)pyridin-3-yl]-N-[(1R)-1-(2-cyanophenyl)ethyl]-6,7-dihydrospiro[pyrazolo[5,1-c][1,4]oxazine-4,3′-pyrrolidine]-1′-carboxamide
2′-[6-amino-5-(trifluoromethyl)pyridin-3-yl]-N-[(1R)-1-(4-fluorophenyl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[2-(4-fluorophenyl)propan-2-yl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[2-(pyridin-3-yl)propan-2-yl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(2-fluorophenyl)methyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[1-(pyridin-3-yl)cyclobutyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(4-fluorophenyl)methyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[2-(pyridin-4-yl)propan-2-yl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[2-(3-fluorophenyl)propan-2-yl]-5′,6′-dihydrospiro[azetidine-3,4′-pyrrolo[1,2-b]pyrazole]-1-carboxamide
2′-[6-amino-5-(trifluoromethyl) pyridin-3-yl]-N-[(1S)-1-(3-chloropyridin-4-yl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(1-methyl-2-oxabicyclo[2.1.1]hexan-4-yl)methyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-{[3-(trifluoromethyl)bicyclo[1.1.1]pentan-1-yl]methyl}-5′,6′-dihydrospiro[azetidine-3,4′-pyrrolo[1,2-b]pyrazole]-1-carboxamide
2′-[6-amino-5-(trifluoromethyl)pyridin-3-yl]-N-[2-(2-chlorophenyl)propan-2-yl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(1R)-1-(3-fluorophenyl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[2-(3-fluoropyridin-2-yl)propan-2-yl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[1-(2-chlorophenyl)cyclobutyl]-5′,6′-dihydrospiro[azetidine-3,4′-pyrrolo[1,2-b]pyrazole]-1-carboxamide
2′-[6-amino-5-(trifluoromethyl) pyridin-3-yl]-N-[1-(3-fluorophenyl)cyclobutyl]-5′,6′-dihydrospiro[azetidine-3,4′-pyrrolo[1,2-b]pyrazole]-1-carboxamide
2′-[6-amino-5-(trifluoromethyl) pyridin-3-yl]-N-[(1R)-1-phenylpropyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(pyrimidin-4-yl)methyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(3-fluoropyridin-2-yl)methyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(pyrimidin-5-yl)methyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(3-chloropyridin-4-yl)methyl]-5′,6′-dihydrospiro[azetidine-3,4′-pyrrolo[1,2-b]pyrazole]-1-carboxamide
2′-[6-amino-5-(trifluoromethyl) pyridin-3-yl]-N-[(3-fluorophenyl)methyl]-5′,6′-dihydrospiro[azetidine-3,4′-pyrrolo[1,2-b]pyrazole]-1-carboxamide
2′-[6-amino-5-(trifluoromethyl) pyridin-3-yl]-N-[(pyridin-3-yl)methyl]-5′,6′-dihydrospiro[azetidine-3,4′-pyrrolo[1,2-b]pyrazole]-1-carboxamide
(rac)-2-[6-amino-5-(trifluoromethyl)pyridin-3-yl]-N-benzyl-6,7-dihydrospiro[pyrazolo[5,1-c][1,4]oxazine-4,3′-pyrrolidine]-1′-carboxamide
(rac)-2-[6-amino-5-(trifluoromethyl)pyridin-3-yl]-N-[(1R)-1-(2-methylpyrimidin-5-yl)ethyl]-6,7-dihydrospiro[pyrazolo[5,1-c][1,4]oxazine-4,3′-pyrrolidine]-1′-carboxamide
(rac)-2-[6-amino-5-(trifluoromethyl)pyridin-3-yl]-N-[(1R)-1-(pyridin-3-yl)ethyl]-6,7-dihydrospiro[pyrazolo[5,1-c][1,4]oxazine-4,3′-pyrrolidine]-1′-carboxamide
2′-[6-amino-5-(trifluoromethyl) pyridin-3-yl]-N-benzyl-5′,6′-dihydrospiro[azetidine-3,4′-pyrrolo[1,2-b]pyrazole]-1-carboxamide
2-[6-amino-5-(trifluoromethyl)pyridin-3-yl]-N-[2-(2-fluorophenyl)propan-2-yl]-6,7-dihydrospiro[pyrazolo[5,1-c][1,4]oxazine-4,3′-pyrrolidine]-1′-carboxamide (enantiomer 1)
2-[6-amino-5-(trifluoromethyl)pyridin-3-yl]-N-[2-(3-chloropyridin-4-yl)propan-2-yl]-6,7-dihydrospiro[pyrazolo[5,1-c][1,4]oxazine-4,3′-pyrrolidine]-1′-carboxamide (enantiomer 1)
2-[6-amino-5-(trifluoromethyl)pyridin-3-yl]-N-[2-(2-fluorophenyl)propan-2-yl]-6,7-dihydrospiro[pyrazolo[5,1-c][1,4]oxazine-4,3′-pyrrolidine]-1′-carboxamide (enantiomer 2)
2-[6-amino-5-(trifluoromethyl)pyridin-3-yl]-N-[2-(3-chloropyridin-4-yl)propan-2-yl]-6,7-dihydrospiro[pyrazolo[5,1-c][1,4]oxazine-4,3′-pyrrolidine]-1′-carboxamide (enantiomer 2)
2-[6-amino-5-(trifluoromethyl)pyridin-3-yl]-N-[(1R)-1-(4-cyanopyridin-2-yl)ethyl]-6,7-dihydrospiro[pyrazolo[5,1-c][1,4]oxazine-4,3′-pyrrolidine]-1′-carboxamide
(rac)-2-[6-amino-5-(trifluoromethyl) pyridin-3-yl]-N-[2-(1-methyl-1H-1,2,3-triazol-5-yl)propan-2-yl]-6,7-dihydrospiro[pyrazolo[5,1-c][1,4]oxazine-4,3′-pyrrolidine]-1′-carboxamide
(rac)-2-[6-amino-5-(trifluoromethyl)pyridin-3-yl]-N-[2-(pyridin-3-yl)propan-2-yl]-6,7-dihydrospiro[pyrazolo[5,1-c][1,4]oxazine-4,3′-pyrrolidine]-1′-carboxamide
2-[6-amino-5-(trifluoromethyl)pyridin-3-yl]-N-[(1R)-1-(4-fluorophenyl)ethyl]-6,7-dihydrospiro[pyrazolo[5,1-c][1,4]oxazine-4,3′-pyrrolidine]-1′-carboxamide
2-[6-amino-5-(trifluoromethyl)pyridin-3-yl]-N-[(1R)-2,3-dihydro-1H-inden-1-yl]-6,7-dihydrospiro[pyrazolo[5,1-c][1,4]oxazine-4,3′-pyrrolidine]-1′-carboxamide
2-[6-amino-5-(trifluoromethyl)pyridin-3-yl]-N-[(1R)-1-(5-fluoropyridin-3-yl)ethyl]-6,7-dihydrospiro[pyrazolo[5,1-c][1,4]oxazine-4,3′-pyrrolidine]-1′-carboxamide
2-[6-amino-5-(trifluoromethyl)pyridin-3-yl]-N-[(1R)-1-(3-fluorophenyl)ethyl]-6,7-dihydrospiro[pyrazolo[5,1-c][1,4]oxazine-4,3′-pyrrolidine]-1′-carboxamide
2-[6-amino-5-(trifluoromethyl)pyridin-3-yl]-N-[(1R)-1-phenylpropyl]-6,7-dihydrospiro[pyrazolo[5,1-c][1,4]oxazine-4,3′-pyrrolidine]-1′-carboxamide
(3′RS)-2-[6-amino-5-(trifluoromethyl)pyridin-3-yl]-N-[(3S)-2,3-dihydro-1-benzofuran-3-yl]-6,7-dihydrospiro[pyrazolo[5,1-c][1,4]oxazine-4,3′-pyrrolidine]-1′-carboxamide
(rac)-2-[6-amino-5-(trifluoromethyl)pyridin-3-yl]-N-[(bicyclo[1.1.1]pentan-1-yl)methyl]-6,7-dihydrospiro[pyrazolo[5,1-c][1,4]oxazine-4,3′-pyrrolidine]-1′-carboxamide
2-[6-amino-5-(trifluoromethyl)pyridin-3-yl]-N-[(1S)-2-methoxy-1-phenylethyl]-6,7-dihydrospiro[pyrazolo[5,1-c][1,4]oxazine-4,3′-pyrrolidine]-1′-carboxamide
2′-[6-amino-5-(trifluoromethyl)pyridin-3-yl]-N-[(1R)-1-(1,2-oxazol-3-yl)ethyl]-5′,6′-dihydrospiro[azetidine-3,4′-pyrrolo[1,2-b]pyrazole]-1-carboxamide
2-[6-amino-5-(trifluoromethyl)pyridin-3-yl]-N-[(1R)-1-(2-fluorophenyl)ethyl]-6,7-dihydrospiro[pyrazolo[5,1-c][1,4]oxazine-4,3′-pyrrolidine]-1′-carboxamide
(rac)-2-[6-amino-5-(trifluoromethyl)pyridin-3-yl]-N-[2-(3-fluorophenyl)propan-2-yl]-6,7-dihydrospiro[pyrazolo[5,1-c][1,4]oxazine-4,3′-pyrrolidine]-1′-carboxamide
2′-[6-amino-5-(trifluoromethyl)pyridin-3-yl]-N-[(1R)-1-(1-phenyl-1H-pyrazol-4-yl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(1S)-2-methoxy-1-phenylethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(3-fluorobicyclo[1.1.1]pentan-1-yl)methyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(bicyclo[1.1.1]pentan-1-yl)methyl]-5′,6′-dihydrospiro[azetidine-3,4′-pyrrolo[1,2-b]pyrazole]-1-carboxamide
2′-[6-amino-5-(trifluoromethyl) pyridin-3-yl]-N-[(1S)-1-(pyridin-3-yl)ethyl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[(1R)-1-(2-methylpyrimidin-5-yl)ethyl]-5′,6′-dihydrospiro[azetidine-3,4′-pyrrolo[1,2-b]pyrazole]-1-carboxamide
2′-[6-amino-5-(trifluoromethyl) pyridin-3-yl]-N-[2-(1-methyl-1H-1,2,3-triazol-4-yl)propan-2-yl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[2-(3-chloropyridin-4-yl)propan-2-yl]-5′,6′-dihydrospiro[azetidine-3,4′-pyrrolo[1,2-b]pyrazole]-1-carboxamide
2′-[6-amino-5-(trifluoromethyl)pyridin-3-yl]-N-[1-(pyridin-4-yl)cyclobutyl]-5′,6′-dihydrospiro[azetidine-3,4′-pyrrolo[1,2-b]pyrazole]-1-carboxamide
2′-[6-amino-5-(trifluoromethyl) pyridin-3-yl]-N-[(1R)-1-phenylethyl]-5′,6′-dihydrospiro[azetidine-3,4′-pyrrolo[1,2-b]pyrazole]-1-carboxamide
2-[6-amino-5-(trifluoromethyl)pyridin-3-yl]-N-[(1S)-1-(pyridin-3-yl)ethyl]-6,7-dihydrospiro[pyrazolo[5,1-c][1,4]oxazine-4,3′-pyrrolidine]-1′-carboxamide
(rac)-2-[6-amino-5-(trifluoromethyl)pyridin-3-yl]-N-[(3-fluorobicyclo[1.1.1]pentan-1-yl)methyl]-6,7-dihydrospiro[pyrazolo[5,1-c][1,4]oxazine-4,3′-pyrrolidine]-1′-carboxamide
2-[6-amino-5-(trifluoromethyl)pyridin-3-yl]-N-[(1S)-1-(pyridin-4-yl)ethyl]-6,7-dihydrospiro[pyrazolo[5,1-c][1,4]oxazine-4,3′-pyrrolidine]-1′-carboxamide
2′-[6-amino-5-(trifluoromethyl) pyridin-3-yl]-N-(1-phenylcyclobutyl)-5′,6′-dihydrospiro[azetidine-3,4′-pyrrolo[1,2-b]pyrazole]-1-carboxamide
2′-(6-amino-5-cyanopyridin-3-yl)-N-[1-(pyridin-4-yl)cyclobutyl]-5′,6′-dihydrospiro[azetidine-3,4′-pyrrolo[1,2-b]pyrazole]-1-carboxamide
2′-(6-amino-5-cyanopyridin-3-yl)-N-[1-(3-fluorophenyl)cyclobutyl]-5′,6′-dihydrospiro[azetidine-3,4′-pyrrolo[1,2-b]pyrazole]-1-carboxamide
2′-(6-amino-5-cyanopyridin-3-yl)-N-[2-(3-chloropyridin-4-yl)propan-2-yl]-5′,6′-dihydrospiro[azetidine-3,4′-pyrrolo[1,2-b]pyrazole]-1-carboxamide
2′-(6-amino-5-cyanopyridin-3-yl)-N-[(1R)-1-(5-fluoropyridin-3-yl)ethyl]-5′,6′-dihydrospiro[azetidine-3,4′-pyrrolo[1,2-b]pyrazole]-1-carboxamide
2′-(6-amino-5-cyanopyridin-3-yl)-N-[(1R)-1-(4-fluorophenyl)ethyl]-5′,6′-dihydrospiro[azetidine-3,4′-pyrrolo[1,2-b]pyrazole]-1-carboxamide
2′-(6-amino-5-cyanopyridin-3-yl)-N-[2-(3-fluorophenyl)propan-2-yl]-5′,6′-dihydrospiro[azetidine-3,4′-pyrrolo[1,2-b]pyrazole]-1-carboxamide
2′-(6-amino-5-cyanopyridin-3-yl)-N-[(1S)-1-(3-chloropyridin-4-yl)ethyl]-5′,6′-dihydrospiro[azetidine-3,4′-pyrrolo[1,2-b]pyrazole]-1-carboxamide
2′-(6-amino-5-cyanopyridin-3-yl)-N-[(1-methyl-2-oxabicyclo[2.1.1]hexan-4-yl)methyl]-5′,6′-dihydrospiro[azetidine-3,4′-pyrrolo[1,2-b]pyrazole]-1-carboxamide
2′-(6-amino-5-cyanopyridin-3-yl)-N-[(1R)-1-(4-cyanopyridin-2-yl)ethyl]-5′,6′-dihydrospiro[azetidine-3,4′-pyrrolo[1,2-b]pyrazole]-1-carboxamide
2′-(6-amino-5-cyanopyridin-3-yl)-N-[(1R)-1-(2-cyanopyridin-4-yl)ethyl]-5′,6′-dihydrospiro[azetidine-3,4′-pyrrolo[1,2-b]pyrazole]-1-carboxamide
2′-(6-amino-5-cyanopyridin-3-yl)-N-[(1R)-1-(2-chloro-4-cyanophenyl)ethyl]-5′,6′-dihydrospiro[azetidine-3,4′-pyrrolo[1,2-b]pyrazole]-1-carboxamide
2′-(6-amino-5-cyanopyridin-3-yl)-N-[1-(2-fluorophenyl)cyclobutyl]-5′,6′-dihydrospiro[azetidine-3,4′-pyrrolo[1,2-b]pyrazole]-1-carboxamide
2′-(6-amino-5-cyanopyridin-3-yl)-N-[2-(4-fluorophenyl)propan-2-yl]-5′,6′-dihydrospiro[azetidine-3,4′-pyrrolo[1,2-b]pyrazole]-1-carboxamide
2′-(6-amino-5-cyanopyridin-3-yl)-N-[1-(2-chlorophenyl)cyclobutyl]-5′,6′-dihydrospiro[azetidine-3,4′-pyrrolo[1,2-b]pyrazole]-1-carboxamide
2′-(6-amino-5-cyanopyridin-3-yl)-N-[2-(1-methyl-1H-1,2,3-triazol-4-yl)propan-2-yl]-5′,6′-dihydrospiro[azetidine-3,4′-pyrrolo[1,2-b]pyrazole]-1-carboxamide
2′-(6-amino-5-cyanopyridin-3-yl)-N-[2-(pyridin-2-yl)propan-2-yl]-5′,6′-dihydrospiro[azetidine-3,4′-pyrrolo[1,2-b]pyrazole]-1-carboxamide
2′-(6-amino-5-cyanopyridin-3-yl)-N-[2-(pyridin-4-yl)propan-2-yl]-5′,6′-dihydrospiro[azetidine-3,4′-pyrrolo[1,2-b]pyrazole]-1-carboxamide
2′-(6-amino-5-cyanopyridin-3-yl)-N-[(1R)-1-phenylpropyl]-5′,6′-dihydrospiro[azetidine-3,4′-pyrrolo[1,2-b]pyrazole]-1-carboxamide
2′-(6-amino-5-cyanopyridin-3-yl)-N-[(1R)-1-(3-fluorophenyl)ethyl]-5′,6′-dihydrospiro[azetidine-3,4′-pyrrolo[1,2-b]pyrazole]-1-carboxamide
2′-(6-amino-5-cyanopyridin-3-yl)-N-[(1R)-1-(2-cyanophenyl)ethyl]-5′,6′-dihydrospiro[azetidine-3,4′-pyrrolo[1,2-b]pyrazole]-1-carboxamide
(rac)-2′-(6-amino-5-cyanopyridin-3-yl)-N-[2-(pyridin-4-yl)propan-2-yl]-5′,6′-dihydrospiro[pyrrolidine-3,4′-pyrrolo[1,2-b]pyrazole]-1-carboxamide
2′-(6-amino-5-cyanopyridin-3-yl)-N-[(1R)-1-(5-fluoropyridin-3-yl)ethyl]-5′,6′-dihydrospiro[pyrrolidine-3,4′-pyrrolo[1,2-b]pyrazole]-1-carboxamide
(rac)-2′-(6-amino-5-cyanopyridin-3-yl)-N-[2-(1-methyl-1H-1,2,3-triazol-4-yl)propan-2-yl]-5′,6′-dihydrospiro[pyrrolidine-3,4′-pyrrolo[1,2-b]pyrazole]-1-carboxamide
(rac)-2-(6-amino-5-cyanopyridin-3-yl)-N-[1-(pyridin-2-yl)cyclobutyl]-6,7-dihydrospiro[pyrazolo[5,1-c][1,4]oxazine-4,3′-pyrrolidine]-1′-carboxamide
(rac)-2′-(6-amino-5-cyanopyridin-3-yl)-N-[1-(pyridin-2-yl)cyclobutyl]-5′,6′-dihydrospiro[pyrrolidine-3,4′-pyrrolo[1,2-b]pyrazole]-1-carboxamide
(rac)-2′-(6-amino-5-cyanopyridin-3-yl)-N-[1-(2-chlorophenyl)cyclobutyl]-5′,6′-dihydrospiro[pyrrolidine-3,4′-pyrrolo[1,2-b]pyrazole]-1-carboxamide
2-(6-amino-5-cyanopyridin-3-yl)-N-[(1R)-1-phenylethyl]-6,7-dihydrospiro[pyrazolo[5,1-c][1,4]oxazine-4,3′-pyrrolidine]-1′-carboxamide
2′-(6-amino-5-cyanopyridin-3-yl)-N-[(1R)-1-phenylethyl]-5′,6′-dihydrospiro[pyrrolidine-3,4′-pyrrolo[1,2-b]pyrazole]-1-carboxamide
(rac)-2-(6-amino-5-cyanopyridin-3-yl)-N-[1-(2-chlorophenyl)cyclobutyl]-6,7-dihydrospiro[pyrazolo[5,1-c][1,4]oxazine-4,3′-pyrrolidine]-1′-carboxamide
(rac)-2-(6-amino-5-cyanopyridin-3-yl)-N-[1-(4-fluorophenyl)cyclobutyl]-6,7-dihydrospiro[pyrazolo[5,1-c][1,4]oxazine-4,3′-pyrrolidine]-1′-carboxamide
(rac)-2-(6-amino-5-cyanopyridin-3-yl)-N-[2-(4-fluorophenyl)propan-2-yl]-6,7-dihydrospiro[pyrazolo[5,1-c][1,4]oxazine-4,3′-pyrrolidine]-1′-carboxamide
(rac)-2-(6-amino-5-cyanopyridin-3-yl)-N-[2-(pyridin-2-yl)propan-2-yl]-6,7-dihydrospiro[pyrazolo[5,1-c][1,4]oxazine-4,3′-pyrrolidine]-1′-carboxamide
(rac)-2′-(6-amino-5-cyanopyridin-3-yl)-N-[2-(pyridin-2-yl)propan-2-yl]-5′,6′-dihydrospiro[pyrrolidine-3,4′-pyrrolo[1,2-b]pyrazole]-1-carboxamide
2-(6-amino-5-cyanopyridin-3-yl)-N-[1-(pyridin-4-yl)cyclobutyl]-6,7-dihydrospiro[pyrazolo[5,1-c][1,4]oxazine-4,3′-pyrrolidine]-1′-carboxamide (enantiomer 2)
2-(6-amino-5-cyanopyridin-3-yl)-N-[1-(2-chlorophenyl)cyclobutyl]-6,7-dihydrospiro[pyrazolo[5,1-c][1,4]oxazine-4,3′-pyrrolidine]-1′-carboxamide (enantiomer 2)
2-(6-amino-5-cyanopyridin-3-yl)-N-[2-(pyridin-2-yl)propan-2-yl]-6,7-dihydrospiro[pyrazolo[5,1-c][1,4]oxazine-4,3′-pyrrolidine]-1′-carboxamide (enantiomer 1)
2-(6-amino-5-cyanopyridin-3-yl)-N-[1-(2-chlorophenyl)cyclobutyl]-6,7-dihydrospiro[pyrazolo[5,1-c][1,4]oxazine-4,3′-pyrrolidine]-1′-carboxamide (enantiomer 1)
2′-(6-amino-5-cyanopyridin-3-yl)-N-[1-(pyridin-3-yl)cyclobutyl]-5′,6′-dihydrospiro[azetidine-3,4′-pyrrolo[1,2-b]pyrazole]-1-carboxamide
2′-(6-amino-5-cyanopyridin-3-yl)-N-[1-(pyridin-2-yl)cyclobutyl]-5′,6′-dihydrospiro[azetidine-3,4′-pyrrolo[1,2-b]pyrazole]-1-carboxamide
2-(6-amino-5-cyanopyridin-3-yl)-N-[1-(pyridin-4-yl)cyclobutyl]-6,7-dihydrospiro[pyrazolo[5,1-c][1,4]oxazine-4,3′-pyrrolidine]-1′-carboxamide (enantiomer 1)
2′-(6-amino-5-cyanopyridin-3-yl)-N-(1-phenylcyclobutyl)-5′,6′-dihydrospiro[azetidine-3,4′-pyrrolo[1,2-b]pyrazole]-1-carboxamide
2′-(6-amino-5-cyanopyridin-3-yl)-N-[(3S)-2,3-dihydro-1-benzofuran-3-yl]-5′,6′-dihydrospiro[pyrrolidine-3,4′-pyrrolo[1,2-b]pyrazole]-1-carboxamide
2-(6-amino-5-cyanopyridin-3-yl)-N-[(3S)-2,3-dihydro-1-benzofuran-3-yl]-6,7-dihydrospiro[pyrazolo[5,1-c][1,4]oxazine-4,3′-pyrrolidine]-1′-carboxamide
2-(6-amino-5-cyanopyridin-3-yl)-N-[(1R)-1-(2-fluorophenyl)ethyl]-6,7-dihydrospiro[pyrazolo[5,1-c][1,4]oxazine-4,3′-pyrrolidine]-1′-carboxamide
2′-(6-amino-5-cyanopyridin-3-yl)-N-[(1R)-1-(2-fluorophenyl)ethyl]-5′,6′-dihydrospiro[pyrrolidine-3,4′-pyrrolo[1,2-b]pyrazole]-1-carboxamide
(rac)-2′-(6-amino-5-cyanopyridin-3-yl)-N-[2-(4-fluorophenyl)propan-2-yl]-5′,6′-dihydrospiro[pyrrolidin-3,4′-pyrrolo[1,2-b]pyrazole]-1-carboxamide
(rac)-2-(6-amino-5-cyanopyridin-3-yl)-N-[2-(1-methyl-1H-1,2,3-triazol-4-yl)propan-2-yl]-6,7-dihydrospiro[pyrazolo[5,1-c][1,4]oxazine-4,3′-pyrrolidine]-1′-carboxamide
(rac)-2-(6-amino-5-cyanopyridin-3-yl)-N-[(1R)-1-(5-fluoropyridin-3-yl)ethyl]-6,7-dihydrospiro[pyrazolo[5,1-c][1,4]oxazine-4,3′-pyrrolidine]-1′-carboxamide
(rac)-2′-(6-amino-5-cyanopyridin-3-yl)-N-(1-phenylcyclobutyl)-5′,6′-dihydrospiro[pyrrolidine-3,4′-pyrrolo[1,2-b]pyrazole]-1-carboxamide
(rac)-2′-(6-amino-5-cyanopyridin-3-yl)-N-[1-(4-fluorophenyl)cyclobutyl]-5′,6′-dihydrospiro[pyrrolidine-3,4′-pyrrolo[1,2-b]pyrazole]-1-carboxamide
(rac)-2-(6-amino-5-cyanopyridin-3-yl)-N-[1-(pyridin-4-yl)cyclobutyl]-6,7-dihydrospiro[pyrazolo[5,1-c][1,4]oxazine-4,3′-pyrrolidine]-1′-carboxamide
(rac)-2′-(6-amino-5-cyanopyridin-3-yl)-N-[1-(pyridin-4-yl)cyclobutyl]-5′,6′-dihydrospiro[pyrrolidine-3,4′-pyrrolo[1,2-b]pyrazole]-1-carboxamide
(rac)-2-(6-amino-5-cyanopyridin-3-yl)-N-(1-phenylcyclobutyl)-6,7-dihydrospiro[pyrazolo[5,1-c][1,4]oxazine-4,3′-pyrrolidine]-1′-carboxamide
2′-(6-amino-5-cyanopyridin-3-yl)-N-[(1R)-1-(1,3,4-trimethyl-1H-pyrazol-5-yl)ethyl]-5′,6′-dihydrospiro[azetidine-3,4′-pyrrolo[1,2-b]pyrazole]-1-carboxamide
(rac)-2-(6-amino-5-cyanopyridin-3-yl)-N-[2-(pyridin-4-yl)propan-2-yl]-6,7-dihydrospiro[pyrazolo[5,1-c][1,4]oxazine-4,3′-pyrrolidine]-1′-carboxamide
2′-[6-amino-5-(difluoromethoxy)pyridin-3-yl]-N-[1-(pyridin-3-yl)cyclobutyl]-5′,6′-dihydrospiro[azetidine-3,4′-pyrrolo[1,2-b]pyrazole]-1-carboxamide
(rac)-2′-[6-amino-5-(trifluoromethoxy)pyridin-3-yl]-N-[2-(1-methyl-1H-1,2,3-triazol-4-yl)propan-2-yl]-5′,6′-dihydrospiro[pyrrolidine-3,4′-pyrrolo[1,2-b]pyrazole]-1-carboxamide
2′-[6-amino-5-(trifluoromethoxy)pyridin-3-yl]-N-[(1R)-1-(2-methylpyrimidin-5-yl)ethyl]-5′,6′-dihydrospiro[pyrrolidine-3,4′-pyrrolo[1,2-b]pyrazole]-1-carboxamide
2′-[6-amino-5-(trifluoromethoxy)pyridin-3-yl]-N-[(1R)-1-(pyridin-2-yl)ethyl]-5′,6′-dihydrospiro[pyrrolidine-3,4′-pyrrolo[1,2-b]pyrazole]-1-carboxamide
2′-[6-amino-5-(trifluoromethoxy)pyridin-3-yl]-N-[(1R)-1-phenylethyl]-5′,6′-dihydrospiro[pyrrolidine-3,4′-pyrrolo[1,2-b]pyrazole]-1-carboxamide (mixture of stereoisomer) 2′-[6-amino-5-(trifluoromethoxy)pyridin-3-yl]-N-[(1R)-1-(pyridin-3-yl)ethyl]-5′,6′-dihydrospiro[pyrrolidine-3,4′-pyrrolo[1,2-b]pyrazole]-1-carboxamide
2′-[6-amino-5-(trifluoromethoxy)pyridin-3-yl]-N-[(1S)-1-(pyridin-4-yl)ethyl]-5′,6′-dihydrospiro[pyrrolidine-3,4′-pyrrolo[1,2-b]pyrazole]-1-carboxamide
(rac)-2′-[6-amino-5-(trifluoromethoxy)pyridin-3-yl]-N-[2-(4-fluorophenyl)propan-2-yl]-5′,6′-dihydrospiro[pyrrolidine-3,4′-pyrrolo[1,2-b]pyrazole]-1-carboxamide
2′-[6-amino-5-(trifluoromethoxy)pyridin-3-yl]-N-[(1R)-1-(4-fluorophenyl)ethyl]-5′,6′-dihydrospiro[pyrrolidine-3,4′-pyrrolo[1,2-b]pyrazole]-1-carboxamide
(rac)-2-[6-amino-5-(trifluoromethoxy)pyridin-3-yl]-N-[2-(pyridin-4-yl)propan-2-yl]-6,7-dihydrospiro[pyrazolo[5,1-c][1,4]oxazine-4,3′-pyrrolidine]-1′-carboxamide
(rac)-2-[6-amino-5-(trifluoromethoxy)pyridin-3-yl]-N-[1-(pyridin-4-yl)cyclobutyl]-6,7-dihydrospiro[pyrazolo[5,1-c][1,4]oxazine-4,3′-pyrrolidine]-1′-carboxamide
2′-[6-amino-5-(difluoromethoxy)pyridin-3-yl]-N-(1-phenylcyclobutyl)-5′,6′-dihydrospiro[azetidine-3,4′-pyrrolo[1,2-b]pyrazole]-1-carboxamide
(rac)-2′-[6-amino-5-(trifluoromethoxy)pyridin-3-yl]-N-[2-(pyridin-2-yl)propan-2-yl]-5′,6′-dihydrospiro[pyrrolidine-3,4′-pyrrolo[1,2-b]pyrazole]-1-carboxamide
(rac)-2′-[6-amino-5-(trifluoromethoxy)pyridin-3-yl]-N-[2-(3-chloropyridin-4-yl)propan-2-yl]-5′,6′-dihydrospiro[pyrrolidine-3,4′-pyrrolo[1,2-b]pyrazole]-1-carboxamide
2′-[6-amino-5-(difluoromethoxy)pyridin-3-yl]-N-[2-(2-fluorophenyl)propan-2-yl]-5′,6′-dihydrospiro[azetidine-3,4′-pyrrolo[1,2-b]pyrazole]-1-carboxamide
(rac)-2-[6-amino-5-(difluoromethoxy)pyridin-3-yl]-N-[2-(3-fluorophenyl)propan-2-yl]-6,7-dihydrospiro[pyrazolo[5,1-c][1,4]oxazine-4,3′-pyrrolidine]-1′-carboxamide
2-[6-amino-5-(difluoromethoxy)pyridin-3-yl]-N-[(1S)-1-(3-chloropyridin-4-yl)ethyl]-6,7-dihydrospiro[pyrazolo[5,1-c][1,4]oxazine-4,3′-pyrrolidine]-1′-carboxamide
2-[6-amino-5-(trifluoromethoxy)pyridin-3-yl]-N-[1-(1,3,5-trimethyl-1H-pyrazol-4-yl)ethyl]-6,7-dihydrospiro[pyrazolo[5,1-c][1,4]oxazine-4,3′-pyrrolidine]-1′-carboxamide
2-[6-amino-5-(trifluoromethoxy)pyridin-3-yl]-N-[1-(1-ethyl-3,5-dimethyl-1H-pyrazol-4-yl)ethyl]-6,7-dihydrospiro[pyrazolo[5,1-c][1,4]oxazine-4,3′-pyrrolidine]-1′-carboxamide
2-[6-amino-5-(trifluoromethoxy)pyridin-3-yl]-N-[(1R)-1-(2-cyanophenyl)ethyl]-6,7-dihydrospiro[pyrazolo[5,1-c][1,4]oxazine-4,3′-pyrrolidine]-1′-carboxamide
(rac)-2′-[6-amino-5-(difluoromethoxy)pyridin-3-yl]-N-[2-(pyridin-2-yl)propan-2-yl]-5′,6′-dihydrospiro[pyrrolidine-3,4′-pyrrolo[1,2-b]pyrazole]-1-carboxamide
2-[6-amino-5-(trifluoromethoxy)pyridin-3-yl]-N-[(1R)-1-(4-fluorophenyl)ethyl]-6,7-dihydrospiro[pyrazolo[5,1-c][1,4]oxazine-4,3′-pyrrolidine]-1′-carboxamide
2-[6-amino-5-(trifluoromethoxy)pyridin-3-yl]-N-[(1R)-1-phenylpropyl]-6,7-dihydrospiro[pyrazolo[5,1-c][1,4]oxazine-4,3′-pyrrolidine]-1′-carboxamide
2-[6-amino-5-(trifluoromethoxy)pyridin-3-yl]-N-[(1R)-1-phenylethyl]-6,7-dihydrospiro[pyrazolo[5,1-c][1,4]oxazine-4,3′-pyrrolidine]-1′-carboxamide
(rac)-2-[6-amino-5-(trifluoromethoxy)pyridin-3-yl]-N-[1-(pyridin-3-yl)cyclobutyl]-6,7-dihydrospiro[pyrazolo[5,1-c][1,4]oxazine-4,3′-pyrrolidine]-1′-carboxamide
2-[6-amino-5-(difluoromethoxy)pyridin-3-yl]-N-[(1R)-1-(2-chloro-4-cyanophenyl)ethyl]-6,7-dihydrospiro[pyrazolo[5,1-c][1,4]oxazine-4,3′-pyrrolidine]-1′-carboxamide
(rac)-2-[6-amino-5-(difluoromethoxy)pyridin-3-yl]-N-[2-(4-fluorophenyl)propan-2-yl]-6,7-dihydrospiro[pyrazolo[5,1-c][1,4]oxazine-4,3′-pyrrolidine]-1′-carboxamide
(rac)-2-[6-amino-5-(difluoromethoxy)pyridin-3-yl]-N-[1-(2-fluorophenyl)cyclobutyl]-6,7-dihydrospiro[pyrazolo[5,1-c][1,4]oxazine-4,3′-pyrrolidine]-1′-carboxamide
(rac)-2′-[6-amino-5-(difluoromethoxy)pyridin-3-yl]-N-[2-(pyridin-3-yl)propan-2-yl]-5′,6′-dihydrospiro[pyrrolidine-3,4′-pyrrolo[1,2-b]pyrazole]-1-carboxamide
2′-[6-amino-5-(difluoromethoxy)pyridin-3-yl]-N-[1-(2-chlorophenyl)cyclobutyl]-5′,6′-dihydrospiro[azetidine-3,4′-pyrrolo[1,2-b]pyrazole]-1-carboxamide
2′-[6-amino-5-(trifluoromethoxy)pyridin-3-yl]-N-[(1R)-1-(4-cyanophenyl)ethyl]-5′,6′-dihydrospiro[azetidine-3,4′-pyrrolo[1,2-b]pyrazole]-1-carboxamide
2′-[6-amino-5-(difluoromethoxy)pyridin-3-yl]-N-[(1R)-1-(1,3,4-trimethyl-1H-pyrazol-5-yl)ethyl]-5′,6′-dihydrospiro[azetidine-3,4′-pyrrolo[1,2-b]pyrazole]-1-carboxamide
(rac)-2′-[6-amino-5-(trifluoromethoxy)pyridin-3-yl]-N-[1-(2-chlorophenyl)cyclobutyl]-5′,6′-dihydrospiro[pyrrolidine-3,4′-pyrrolo[1,2-b]pyrazole]-1-carboxamide
2′-[6-amino-5-(difluoromethoxy)pyridin-3-yl]-N-[(1R)-1-(5-fluoropyridin-3-yl)ethyl]-5′,6′-dihydrospiro[azetidine-3,4′-pyrrolo[1,2-b]pyrazole]-1-carboxamide
2′-[6-amino-5-(trifluoromethoxy)pyridin-3-yl]-N-[(1R)-1-(4-fluorophenyl)ethyl]-5′,6′-dihydrospiro[azetidine-3,4′-pyrrolo[1,2-b]pyrazole]-1-carboxamide
2′-[6-amino-5-(trifluoromethoxy)pyridin-3-yl]-N-[(1R)-1-(2-chloro-4-cyanophenyl)ethyl]-5′,6′-dihydrospiro[pyrrolidine-3,4′-pyrrolo[1,2-b]pyrazole]-1-carboxamide
2′-[6-amino-5-(difluoromethoxy)pyridin-3-yl]-N-[(1R)-1-(2-cyanophenyl)ethyl]-5′,6′-dihydrospiro[azetidine-3,4′-pyrrolo[1,2-b]pyrazole]-1-carboxamide
2′-[6-amino-5-(difluoromethoxy)pyridin-3-yl]-N-[2-(pyridin-4-yl)propan-2-yl]-5′,6′-dihydrospiro[azetidine-3,4′-pyrrolo[1,2-b]pyrazole]-1-carboxamide
2′-[6-amino-5-(difluoromethoxy)pyridin-3-yl]-N-[(1R)-1-(4-fluorophenyl)ethyl]-5′,6′-dihydrospiro[azetidine-3,4′-pyrrolo[1,2-b]pyrazole]-1-carboxamide
2′-[6-amino-5-(difluoromethoxy)pyridin-3-yl]-N-[(1R)-1-(2-cyanopyridin-4-yl)ethyl]-5′,6′-dihydrospiro[azetidine-3,4′-pyrrolo[1,2-b]pyrazole]-1-carboxamide
(rac)-2′-[6-amino-5-(trifluoromethoxy)pyridin-3-yl]-N-[1-(2-fluorophenyl)cyclobutyl]-5′,6′-dihydrospiro[pyrrolidine-3,4′-pyrrolo[1,2-b]pyrazole]-1-carboxamide
(rac)-2-[6-amino-5-(trifluoromethoxy)pyridin-3-yl]-N-[2-(2-chlorophenyl)propan-2-yl]-6,7-dihydrospiro[pyrazolo[5,1-c][1,4]oxazine-4,3′-pyrrolidine]-1′-carboxamide
(rac)-2-[6-amino-5-(trifluoromethoxy)pyridin-3-yl]-N-[2-(4-fluorophenyl)propan-2-yl]-6,7-dihydrospiro[pyrazolo[5,1-c][1,4]oxazine-4,3′-pyrrolidine]-1′-carboxamide
(rac)-2′-[6-amino-5-(trifluoromethoxy)pyridin-3-yl]-N-[2-(3-fluorophenyl)propan-2-yl]-5′,6′-dihydrospiro[pyrrolidine-3,4′-pyrrolo[1,2-b]pyrazole]-1-carboxamide
2′-[6-amino-5-(trifluoromethoxy)pyridin-3-yl]-N-[(1S)-1-(3-chloropyridin-4-yl)ethyl]-5′,6′-dihydrospiro[pyrrolidine-3,4′-pyrrolo[1,2-b]pyrazole]-1-carboxamide
2′-[6-amino-5-(difluoromethoxy)pyridin-3-yl]-N-[2-(4-fluorophenyl)propan-2-yl]-5′,6′-dihydrospiro[azetidine-3,4′-pyrrolo[1,2-b]pyrazole]-1-carboxamide
2′-[6-amino-5-(difluoromethoxy)pyridin-3-yl]-N-[2-(2-chlorophenyl)propan-2-yl]-5′,6′-dihydrospiro[azetidine-3,4′-pyrrolo[1,2-b]pyrazole]-1-carboxamide
2-[6-amino-5-(trifluoromethoxy)pyridin-3-yl]-N-[(1R)-1-(3-fluorophenyl)ethyl]-6,7-dihydrospiro[pyrazolo[5,1-c][1,4]oxazine-4,3′-pyrrolidine]-1′-carboxamide
2′-[6-amino-5-(trifluoromethoxy)pyridin-3-yl]-N-(1-phenylcyclobutyl)-5′,6′-dihydrospiro[azetidine-3,4′-pyrrolo[1,2-b]pyrazole]-1-carboxamide
2′-[6-amino-5-(trifluoromethoxy)pyridin-3-yl]-N-benzyl-5′,6′-dihydrospiro[azetidine-3,4′-pyrrolo[1,2-b]pyrazole]-1-carboxamide
2′-[6-amino-5-(trifluoromethoxy)pyridin-3-yl]-N-[(1R)-1-(4-cyanopyridin-2-yl)ethyl]-5′,6′-dihydrospiro[azetidine-3,4′-pyrrolo[1,2-b]pyrazole]-1-carboxamide
2′-[6-amino-5-(trifluoromethoxy)pyridin-3-yl]-N-[(1R)-1-(2-cyanopyridin-4-yl)ethyl]-5′,6′-dihydrospiro[azetidine-3,4′-pyrrolo[1,2-b]pyrazole]-1-carboxamide
2′-[6-amino-5-(trifluoromethoxy)pyridin-3-yl]-N-[(1R)-1-(5-fluoropyridin-3-yl)ethyl]-5′,6′-dihydrospiro[pyrrolidine-3,4′-pyrrolo[1,2-b]pyrazole]-1-carboxamide
(rac)-2′-[6-amino-5-(trifluoromethoxy)pyridin-3-yl]-N-[2-(pyridin-4-yl)propan-2-yl]-5′,6′-dihydrospiro[pyrrolidine-3,4′-pyrrolo[1,2-b]pyrazole]-1-carboxamide
(rac)-2′-[6-amino-5-(trifluoromethoxy)pyridin-3-yl]-N-(1-phenylcyclobutyl)-5′,6′-dihydrospiro[pyrrolidine-3,4′-pyrrolo[1,2-b]pyrazole]-1-carboxamide
(rac)-2′-[6-amino-5-(trifluoromethoxy)pyridin-3-yl]-N-[1-(pyridin-4-yl)cyclobutyl]-5′,6′-dihydrospiro[pyrrolidine-3,4′-pyrrolo[1,2-b]pyrazole]-1-carboxamide
2′-[6-amino-5-(difluoromethoxy)pyridin-3-yl]-N-[2-(3-fluorophenyl)propan-2-yl]-5′,6′-dihydrospiro[azetidine-3,4′-pyrrolo[1,2-b]pyrazole]-1-carboxamide
2′-[6-amino-5-(difluoromethoxy)pyridin-3-yl]-N-[(1S)-1-(3-chloropyridin-4-yl)ethyl]-5′,6′-dihydrospiro[azetidine-3,4′-pyrrolo[1,2-b]pyrazole]-1-carboxamide
2′-[6-amino-5-(difluoromethoxy)pyridin-3-yl]-N-[2-(3-chloropyridin-4-yl)propan-2-yl]-5′,6′-dihydrospiro[azetidine-3,4′-pyrrolo[1,2-b]pyrazole]-1-carboxamide
2′-[6-amino-5-(difluoromethoxy)pyridin-3-yl]-N-[(1R)-1-(2-chloro-4-cyanophenyl)ethyl]-5′,6′-dihydrospiro[azetidine-3,4′-pyrrolo[1,2-b]pyrazole]-1-carboxamide
2′-[6-amino-5-(difluoromethoxy)pyridin-3-yl]-N-[1-(2-fluorophenyl)cyclobutyl]-5′,6′-dihydrospiro[azetidine-3,4′-pyrrolo[1,2-b]pyrazole]-1-carboxamide
(rac)-2′-[6-amino-5-(trifluoromethoxy)pyridin-3-yl]-N-[2-(2-chlorophenyl)propan-2-yl]-5′,6′-dihydrospiro[pyrrolidine-3,4′-pyrrolo[1,2-b]pyrazole]-1-carboxamide
2-[6-amino-5-(trifluoromethoxy)pyridin-3-yl]-N-[(1R)-1-(4-cyanopyridin-2-yl)ethyl]-6,7-dihydrospiro[pyrazolo[5,1-c][1,4]oxazine-4,3′-pyrrolidine]-1′-carboxamide
(rac)-2-[6-amino-5-(difluoromethoxy)pyridin-3-yl]-N-[1-(2-chlorophenyl)cyclobutyl]-6,7-dihydrospiro[pyrazolo[5,1-c][1,4]oxazine-4,3′-pyrrolidine]-1′-carboxamide
(rac)-2-[6-amino-5-(trifluoromethoxy)pyridin-3-yl]-N-[1-(pyridin-2-yl)cyclobutyl]-6,7-dihydrospiro[pyrazolo[5,1-c][1,4]oxazine-4,3′-pyrrolidine]-1′-carboxamide
2-[6-amino-5-(trifluoromethoxy)pyridin-3-yl]-N-[(1R)-1-(2-cyanopyridin-4-yl)ethyl]-6,7-dihydrospiro[pyrazolo[5,1-c][1,4]oxazine-4,3′-pyrrolidine]-1′-carboxamide
(rac)-2-[6-amino-5-(difluoromethoxy)pyridin-3-yl]-N-[2-(2-chlorophenyl)propan-2-yl]-6,7-dihydrospiro[pyrazolo[5,1-c][1,4]oxazine-4,3′-pyrrolidine]-1′-carboxamide
(rac)-2-[6-amino-5-(trifluoromethoxy)pyridin-3-yl]-N-[1-(2-chlorophenyl)cyclobutyl]-6,7-dihydrospiro[pyrazolo[5,1-c][1,4]oxazine-4,3′-pyrrolidine]-1′-carboxamide
(rac)-2-[6-amino-5-(trifluoromethoxy)pyridin-3-yl]-N-[1-(2-fluorophenyl)cyclobutyl]-6,7-dihydrospiro[pyrazolo[5,1-c][1,4]oxazine-4,3′-pyrrolidine]-1′-carboxamide
(rac)-2-[6-amino-5-(trifluoromethoxy)pyridin-3-yl]-N-[2-(3-chloropyridin-4-yl)propan-2-yl]-6,7-dihydrospiro[pyrazolo[5,1-c][1,4]oxazine-4,3′-pyrrolidine]-1′-carboxamide
(rac)-2′-[6-amino-5-(trifluoromethoxy)pyridin-3-yl]-N-[1-(pyridin-4-yl)cyclobutyl]-5′,6′-dihydro-1H-spiro[pyrrolidine-3,4′-pyrrolo[1,2-b]pyrazole]-1-carboxamide acetate (1:1)
(rac)-2-[6-amino-5-(trifluoromethoxy)pyridin-3-yl]-N-[(1-methyl-2-oxabicyclo[2.1.1]hexan-4-yl)methyl]-6,7-dihydrospiro[pyrazolo[5,1-c][1,4]oxazine-4,3′-pyrrolidine]-1′-carboxamide
(rac)-2-[6-amino-5-(trifluoromethoxy)pyridin-3-yl]-N-[2-(2-fluorophenyl)propan-2-yl]-6,7-dihydrospiro[pyrazolo[5,1-c][1,4]oxazine-4,3′-pyrrolidine]-1′-carboxamide
2-[6-amino-5-(trifluoromethoxy)pyridin-3-yl]-N-[(1R)-1-(2-chloro-4-cyanophenyl)ethyl]-6,7-dihydrospiro[pyrazolo[5,1-c][1,4]oxazine-4,3′-pyrrolidine]-1′-carboxamide
2′-[6-amino-5-(difluoromethoxy)pyridin-3-yl]-N-[2-(pyridin-2-yl)propan-2-yl]-5′,6′-dihydrospiro[azetidine-3,4′-pyrrolo[1,2-b]pyrazole]-1-carboxamide
2′-[6-amino-5-(difluoromethoxy)pyridin-3-yl]-N-[2-(1-methyl-1H-1,2,3-triazol-4-yl)propan-2-yl]-5′,6′-dihydrospiro[azetidine-3,4′-pyrrolo[1,2-b]pyrazole]-1-carboxamide
2-[6-amino-5-(trifluoromethoxy)pyridin-3-yl]-N-[(1S)-2-methoxy-1-phenylethyl]-6,7-dihydrospiro[pyrazolo[5,1-c][1,4]oxazine-4,3′-pyrrolidine]-1′-carboxamide
2-[6-amino-5-(trifluoromethoxy)pyridin-3-yl]-N-[(1S)-1-(3-chloropyridin-4-yl)ethyl]-6,7-dihydrospiro[pyrazolo[5,1-c][1,4]oxazine-4,3′-pyrrolidine]-1′-carboxamide
(rac)-2-[6-amino-5-(trifluoromethoxy)pyridin-3-yl]-N-[2-(3-fluorophenyl)propan-2-yl]-6,7-dihydrospiro[pyrazolo[5,1-c][1,4]oxazine-4,3′-pyrrolidine]-1′-carboxamide
(rac)-2′-[6-amino-5-(trifluoromethoxy)pyridin-3-yl]-N-[(1-methyl-2-oxabicyclo[2.1.1]hexan-4-yl)methyl]-5′,6′-dihydrospiro[pyrrolidine-3,4′-pyrrolo[1,2-b]pyrazole]-1-carboxamide
2′-[6-amino-5-(difluoromethoxy)pyridin-3-yl]-N-[(1R)-1-phenylpropyl]-5′,6′-dihydrospiro[azetidine-3,4′-pyrrolo[1,2-b]pyrazole]-1-carboxamide
2′-[6-amino-5-(trifluoromethoxy)pyridin-3-yl]-N-[(1S)-2-methoxy-1-phenylethyl]-5′,6′-dihydrospiro[pyrrolidine-3,4′-pyrrolo[1,2-b]pyrazole]-1-carboxamide
2′-[6-amino-5-(difluoromethoxy)pyridin-3-yl]-N-[(1R)-1-(3-fluorophenyl)ethyl]-5′,6′-dihydrospiro[azetidine-3,4′-pyrrolo[1,2-b]pyrazole]-1-carboxamide
2′-[6-amino-5-(trifluoromethoxy)pyridin-3-yl]-N-[(1S)-1-(pyridin-3-yl)ethyl]-5′,6′-dihydrospiro[pyrrolidine-3,4′-pyrrolo[1,2-b]pyrazole]-1-carboxamide
(rac)-2′-[6-amino-5-(trifluoromethoxy)pyridin-3-yl]-N-[1-(pyridin-3-yl)cyclobutyl]-5′,6′-dihydrospiro[pyrrolidine-3,4′-pyrrolo[1,2-b]pyrazole]-1-carboxamide
(rac)-2′-[6-amino-5-(trifluoromethoxy)pyridin-3-yl]-N-[1-(3-fluorophenyl)cyclobutyl]-5′,6′-dihydrospiro[pyrrolidine-3,4′-pyrrolo[1,2-b]pyrazole]-1-carboxamide
2′-[6-amino-5-(difluoromethoxy)pyridin-3-yl]-N-[1-(propan-2-yl)cyclobutyl]-5′,6′-dihydrospiro[azetidine-3,4′-pyrrolo[1,2-b]pyrazole]-1-carboxamide
2′-[6-amino-5-(difluoromethoxy)pyridin-3-yl]-N-[1-(pyridin-4-yl)cyclobutyl]-5′,6′-dihydrospiro[azetidine-3,4′-pyrrolo[1,2-b]pyrazole]-1-carboxamide
2′-[6-amino-5-(difluoromethoxy)pyridin-3-yl]-N-(bicyclo[2.2.1]heptan-1-yl)-5′,6′-dihydrospiro[azetidine-3,4′-pyrrolo[1,2-b]pyrazole]-1-carboxamide
2′-[6-amino-5-(difluoromethoxy)pyridin-3-yl]-N-(1-benzylcyclobutyl)-5′,6′-dihydrospiro[azetidine-3,4-pyrrolo[1,2-b]pyrazole]-1-carboxamide
2′-[6-amino-5-(difluoromethoxy)pyridin-3-yl]-N-[1-(4-chlorophenyl)cyclobutyl]-5′,6′-dihydrospiro[azetidine-3,4′-pyrrolo[1,2-b]pyrazole]-1-carboxamide
2′-[6-amino-5-(difluoromethoxy)pyridin-3-yl]-N-(1-methylcyclobutyl)-5′,6′-dihydrospiro[azetidine-3,4′-pyrrolo[1,2-b]pyrazole]-1-carboxamide
(rac)-2-[6-amino-5-(trifluoromethoxy)pyridin-3-yl]-N-[1-(1,3,4-trimethyl-1H-pyrazol-5-yl)ethyl]-6,7-dihydrospiro[pyrazolo[5,1-c][1,4]oxazine-4,3′-pyrrolidine]-1′-carboxamide
2-[6-amino-5-(trifluoromethoxy)pyridin-3-yl]-N-[(1R)-2,3-dihydro-1H-inden-1-yl]-6,7-dihydrospiro[pyrazolo[5,1-c][1,4]oxazine-4,3′-pyrrolidine]-1′-carboxamide
2′-[6-amino-5-(difluoromethoxy)pyridin-3-yl]-N-[1-(pyridin-2-yl)cyclobutyl]-5′,6′-dihydrospiro[azetidine-3,4′-pyrrolo[1,2-b]pyrazole]-1-carboxamide
2′-[6-amino-5-(difluoromethoxy)pyridin-3-yl]-N-[1-(3-fluorophenyl)cyclobutyl]-5′,6′-dihydrospiro[azetidine-3,4′-pyrrolo[1,2-b]pyrazole]-1-carboxamide
(rac)-2′-[6-amino-5-(trifluoromethoxy)pyridin-3-yl]-N-[1-(pyridin-2-yl)cyclobutyl]-5′,6′-dihydrospiro[pyrrolidine-3,4′-pyrrolo[1,2-b]pyrazole]-1-carboxamide
2-[6-amino-5-(difluoromethoxy)pyridin-3-yl]-N-[(1S)-2-methoxy-1-phenylethyl]-6,7-dihydrospiro[pyrazolo[5,1-c][1,4]oxazine-4,3′-pyrrolidine]-1′-carboxamide
2′-[6-amino-5-(difluoromethoxy)pyridin-3-yl]-N-[(1R)-1-phenylethyl]-5′,6′-dihydrospiro[pyrrolidine-3,4′-pyrrolo[1,2-b]pyrazole]-1-carboxamide
(rac)-2′-[6-amino-5-(difluoromethoxy)pyridin-3-yl]-N-[1-(pyridin-4-yl)cyclobutyl]-5′,6′-dihydrospiro[pyrrolidine-3,4′-pyrrolo[1,2-b]pyrazole]-1-carboxamide
(rac)-2′-[6-amino-5-(difluoromethoxy)pyridin-3-yl]-N-(1-phenylcyclobutyl)-5′,6′-dihydrospiro[pyrrolidine-3,4′-pyrrolo[1,2-b]pyrazole]-1-carboxamide
(rac)-2′-[6-amino-5-(difluoromethoxy)pyridin-3-yl]-N-[2-(pyridin-4-yl)propan-2-yl]-5′,6′-dihydrospiro[pyrrolidine-3,4′-pyrrolo[1,2-b]pyrazole]-1-carboxamide
2′-[6-amino-5-(difluoromethoxy)pyridin-3-yl]-N-[(1R)-1-(4-fluorophenyl)ethyl]-5′,6′-dihydrospiro[pyrrolidine-3,4′-pyrrolo[1,2-b]pyrazole]-1-carboxamide
(rac)-2′-[6-amino-5-(difluoromethoxy)pyridin-3-yl]-N-[2-(4-fluorophenyl)propan-2-yl]-5′,6′-dihydrospiro[pyrrolidine-3,4′-pyrrolo[1,2-b]pyrazole]-1-carboxamide
2′-[6-amino-5-(difluoromethoxy)pyridin-3-yl]-N-[(1R)-1-(5-fluoropyridin-3-yl)ethyl]-5′,6′-dihydrospiro[pyrrolidine-3,4′-pyrrolo[1,2-b]pyrazole]-1-carboxamide
2′-{6-amino-5-[(1R)-1-(pyridin-2-yl)ethoxy]pyridin-3-yl}-N-[(1R)-1-phenylethyl]-5′,6′-dihydrospiro[pyrrolidine-3,4′-pyrrolo[1,2-b]pyrazole]-1-carboxamide
2′-{6-amino-5-[(1R)-1-(pyridin-2-yl)ethoxy]pyridin-3-yl}-N-[2-(pyridin-4-yl)propan-2-yl]-5′,6′-dihydrospiro[pyrrolidine-3,4′-pyrrolo[1,2-b]pyrazole]-1-carboxamide
2′-{6-amino-5-[(1S)-1-(pyridin-2-yl)ethoxy]pyridin-3-yl}-N-[(1R)-1-phenylethyl]-5′,6′-dihydrospiro[pyrrolidine-3,4′-pyrrolo[1,2-b]pyrazole]-1-carboxamide
2′-{6-amino-5-[(1S)-1-(pyridin-2-yl)ethoxy]pyridin-3-yl}-N-[2-(pyridin-4-yl)propan-2-yl]-5′,6′-dihydrospiro[pyrrolidine-3,4′-pyrrolo[1,2-b]pyrazole]-1-carboxamide
2′-{6-amino-5-[(trifluoromethyl)sulfanyl]pyridin-3-yl}-N-[(1R)-1-(3-fluorophenyl)ethyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(3R)-2,3-dihydro-1-benzofuran-3-yl]-5′,6′-dihydrospiro[pyrrolidine-3,4′-pyrrolo[1,2-b]pyrazole]-1-carboxamide
2′-{6-amino-5-[(1R)-1-(pyridin-3-yl)ethoxy]pyridin-3-yl}-N-[2-(pyridin-4-yl)propan-2-yl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[1-(1-methyl-1H-imidazol-5-yl)ethyl]-5′,6′-dihydrospiro[pyrrolidine-3,4′-pyrrolo[1,2-b]pyrazole]-1-carboxamide (diastereomeric mixture) 2′-{6-amino-5-[(1R)-1-(pyridin-3-yl)ethoxy]pyridin-3-yl}-N-cyclobutyl-5′,6′-dihydrospiro[pyrrolidine-3,4′-pyrrolo[1,2-b]pyrazole]-1-carboxamide
2′-{6-amino-5-[(1R)-1-(pyridin-3-yl)ethoxy]pyridin-3-yl}-N-[2-(1-methyl-1H-1,2,3-triazol-4-yl)propan-2-yl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[cyclopropyl(pyridin-2-yl)methyl]-5′,6′-dihydrospiro[pyrrolidine-3,4′-pyrrolo[1,2-b]pyrazole]-1-carboxamide (diastereomeric mixture)
2′-{6-amino-5-[(1S)-1-(pyridin-3-yl)ethoxy]pyridin-3-yl}-N-cyclobutyl-5′,6′-dihydrospiro[pyrrolidine-3,4′-pyrrolo[1,2-b]pyrazole]-1-carboxamide
2′-{6-amino-5-[(1S)-1-(pyridin-3-yl)ethoxy]pyridin-3-yl}-N-[2-(pyridin-4-yl)propan-2-yl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[2-(2-cyanophenyl)propan-2-yl]-5′,6′-dihydrospiro[pyrrolidine-3,4′-pyrrolo[1,2-b]pyrazole]-1-carboxamide
[2′-{6-amino-5-[(1R)-1-(pyridin-3-yl)ethoxy]pyridin-3-yl}-5′,6′-dihydrospiro[pyrrolidine-3,4′-pyrrolo[1,2-b]pyrazol]-1-yl](1H-imidazol-1-yl)methanone
(rac)-2′-[6-amino-5-(trifluoromethyl) pyridin-3-yl]-N-(1-methylcyclobutyl)-5′,6′-dihydrospiro[pyrrolidine 3,4′-pyrrolo[1,2-b]pyrazole]-1-carboxamide
(rac)-2′-[6-amino-5-(trifluoromethyl)pyridin-3-yl]-N-(3-methyloxetan-3-yl)-5′,6′-dihydrospiro[pyrrolidine-3,4′-pyrrolo[1,2-b]pyrazole]-1-carboxamide
(rac)-2-[6-amino-5-(trifluoromethoxy)pyridin-3-yl]-N-[2-(3-fluoropyridin-2-yl)propan-2-yl]-6,7-dihydrospiro[pyrazolo[5,1-c][1,4]oxazine-4,3′-pyrrolidine]-1′-carboxamide
2′-{6-amino-5-[(1S)-1-phenylethoxy]pyridin-3-yl}-N-[1-(pyridin-4-yl)cyclobutyl]-5′,6′-dihydrospiro[pyrrolidine-3,4′-pyrrolo[1,2-b]pyrazole]-1-carboxamide
2′-{6-amino-5-[(1S)-1-phenylethoxy]pyridin-3-yl}-N-[2-(3-chloropyridin-4-yl)propan-2-yl]-5′,6′-dihydrospiro[pyrrolidine-3,4′-pyrrolo[1,2-b]pyrazole]-1-carboxamide
2′-{6-amino-5-[(1R)-1-phenylethoxy]pyridin-3-yl}-N-[1-(pyridin-4-yl)cyclobutyl]-5′,6′-dihydrospiro[pyrrolidine-3,4′-pyrrolo[1,2-b]pyrazole]-1-carboxamide
2′-{6-amino-5-[(1R)-1-phenylethoxy]pyridin-3-yl}-N-[2-(3-chloropyridin-4-yl)propan-2-yl]-5′,6′-dihydrospiro[pyrrolidine-3,4′-pyrrolo[1,2-b]pyrazole]-1-carboxamide
(rac)-2-[6-amino-5-(trifluoromethoxy)pyridin-3-yl]-N-[2-(pyridin-2-yl)propan-2-yl]-6,7-dihydrospiro[pyrazolo[5,1-c][1,4]oxazine-4,3′-pyrrolidine]-1′-carboxamide
2′-{6-amino-5-[(1R)-1-(pyridin-2-yl)ethoxy]pyridin-3-yl}-N-[2-(3-chloropyridin-4-yl)propan-2-yl]-5′,6′-dihydrospiro[pyrrolidine-3,4′-pyrrolo[1,2-b]pyrazole]-1-carboxamide
2′-{6-amino-5-[(1R)-1-(pyridin-3-yl)ethoxy]pyridin-3-yl}-N-[2-(3-chloropyridin-4-yl)propan-2-yl]-5′,6′-dihydrospiro[pyrrolidine-3,4′-pyrrolo[1,2-b]pyrazole]-1-carboxamide
2′-{6-amino-5-[(1R)-1-(pyridin-4-yl)ethoxy]pyridin-3-yl}-N-[2-(3-chloropyridin-4-yl)propan-2-yl]-5′,6′-dihydrospiro[pyrrolidine-3,4′-pyrrolo[1,2-b]pyrazole]-1-carboxamide
2′-(6-amino-5-cyanopyridin-3-yl)-N-[(1R)-1-(pyridin-4-yl)ethyl]-5′,6′-dihydrospiro[pyrrolidine-3,4′-pyrrolo[1,2-b]pyrazole]-1-carboxamide (isomer 1)
2′-(6-amino-5-cyanopyridin-3-yl)-N-[(1R)-1-(pyridin-4-yl)ethyl]-5′,6′-dihydrospiro[pyrrolidine-3,4′-pyrrolo[1,2-b]pyrazole]-1-carboxamide (isomer 2)
2′-(6-amino-5-cyanopyridin-3-yl)-N-[1-(pyridin-4-yl)cyclobutyl]-5′,6′-dihydrospiro[pyrrolidine-3,4′-pyrrolo[1,2-b]pyrazole]-1-carboxamide (enantiomer 1)
(3R)-2′-[6-amino-5-(trifluoromethyl)pyridin-3-yl]-N-[(1S)-1-(pyridin-4-yl)ethyl]-5′,6′-dihydrospiro[pyrrolidine-3,4′-pyrrolo[1,2-b]pyrazole]-1-carboxamide
(3S)-2′-[6-amino-5-(trifluoromethyl)pyridin-3-yl]-N-[(1S)-1-(pyridin-4-yl)ethyl]-5′,6′-dihydrospiro[pyrrolidine-3,4′-pyrrolo[1,2-b]pyrazole]-1-carboxamide
2′-(6-amino-5-cyanopyridin-3-yl)-N-[(1S)-1-(pyridin-4-yl)ethyl]-5′,6′-dihydrospiro[pyrrolidine-3,4′-pyrrolo[1,2-b]pyrazole]-1-carboxamide (isomer 1)
2′-(6-amino-5-cyanopyridin-3-yl)-N-[(1S)-1-(pyridin-4-yl)ethyl]-5′,6′-dihydrospiro[pyrrolidine-3,4′-pyrrolo[1,2-b]pyrazole]-1-carboxamide (isomer 2)
2′-[6-amino-5-(trifluoromethyl)pyridin-3-yl]-N-[(1S)-1-(pyridin-4-yl)ethyl]-5′,6′-dihydrospiro[azetidine-3,4′-pyrrolo[1,2-b]pyrazole]-1-carboxamide
2′-[6-amino-5-(trifluoromethyl) pyridin-3-yl]-N-[(1R)-1-(pyridin-4-yl)ethyl]-5′,6′-dihydrospiro[azetidine-3,4′-pyrrolo[1,2-b]pyrazole]-1-carboxamide
(rac)-2′-[6-amino-5-(trifluoromethyl) pyridin-3-yl]-N-[1-(2-chlorophenyl)cyclobutyl]-5′,6′-dihydrospiro[pyrrolidine-3,4′-pyrrolo[1,2-b]pyrazole]-1-carboxamide
(rac)-2-[6-amino-5-(trifluoromethyl)pyridin-3-yl]-N-[2-(2-fluorophenyl)propan-2-yl]-6,7-dihydrospiro[pyrazolo[5,1-c][1,4]oxazine-4,3′-pyrrolidine]-1′-carboxamide
(rac)-2-[6-amino-5-(trifluoromethyl)pyridin-3-yl]-N-(1-phenylcyclobutyl)-6,7-dihydrospiro[pyrazolo[5,1-c][1,4]oxazine-4,3′-pyrrolidine]-1′-carboxamide
(rac)-2′-[6-amino-5-(trifluoromethyl)pyridin-3-yl]-N-[2-(4-fluorophenyl)propan-2-yl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[2-(2-chlorophenyl)propan-2-yl]-5′,6′-dihydrospiro[pyrrolidine-3,4′-pyrrolo[1,2-b]pyrazole]-1-carboxamide
2-[6-amino-5-(trifluoromethyl)pyridin-3-yl]-N-[(1R)-1-(2-chloro-4-cyanophenyl)ethyl]-6,7-dihydrospiro[pyrazolo[5,1-c][1,4]oxazine-4,3′-pyrrolidine]-1′-carboxamide (mixture of stereoisomer)
2′-[6-amino-5-(trifluoromethyl)pyridin-3-yl]-N-[(1R)-1-(2-chloro-4-cyanophenyl)ethyl]-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[(1S)-1-(3-chloropyridin-4-yl)ethyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[2-(3-fluorophenyl)propan-2-yl]-5′,6′-dihydrospiro[pyrrolidine-3,4′-pyrrolo[1,2-b]pyrazole]-1-carboxamide
(rac)-2-[6-amino-5-(trifluoromethyl)pyridin-3-yl]-N-[1-(pyridin-4-yl)cyclobutyl]-6,7-dihydrospiro[pyrazolo[5,1-c][1,4]oxazine-4,3′-pyrrolidine]-1′-carboxamide
(rac)-2′-[6-amino-5-(trifluoromethyl)pyridin-3-yl]-N-[2-(3-chloropyridin-4-yl)propan-2-yl]-5′,6′-dihydrospiro[pyrrolidine-3,4′-pyrrolo[1,2-b]pyrazole]-1-carboxamide
(rac)-2′-[6-amino-5-(trifluoromethyl) pyridin-3-yl]-N-(1-phenylcyclobutyl)-5′,6′-dihydrospiro[pyrrolidine-3,4′-pyrrolo[1,2-b]pyrazole]-1-carboxamide
(rac)-2-[6-amino-5-(trifluoromethyl)pyridin-3-yl]-N-[2-(pyridin-2-yl)propan-2-yl]-6,7-dihydrospiro[pyrazolo[5,1-c][1,4]oxazine-4,3′-pyrrolidine]-1′-carboxamide
(rac)-2-[6-amino-5-(trifluoromethyl)pyridin-3-yl]-N-[1-(2-fluorophenyl)cyclobutyl]-6,7-dihydrospiro[pyrazolo[5,1-c][1,4]oxazine-4,3′-pyrrolidine]-1′-carboxamide
(rac)-2′-[6-amino-5-(trifluoromethyl)pyridin-3-yl]-N-[1-(2-fluorophenyl)cyclobutyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[1-(3-fluorophenyl)cyclobutyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[1-(pyridin-4-yl)cyclobutyl]-5′,6′-dihydrospiro[pyrrolidine-3,4′-pyrrolo[1,2-b]pyrazole]-1-carboxamide
(rac)-2-[6-amino-5-(trifluoromethyl)pyridin-3-yl]-N-[1-(3-fluorophenyl)cyclobutyl]-6,7-dihydrospiro[pyrazolo[5,1-c][1,4]oxazine-4,3′-pyrrolidine]-1′-carboxamide
(3′R)-2-[6-amino-5-(trifluoromethoxy)pyridin-3-yl]-N-[2-(pyridin-2-yl)propan-2-yl]-6,7-dihydrospiro[pyrazolo[5,1-c][1,4]oxazine-4,3′-pyrrolidine]-1′-carboxamide
(3′R)-2-[6-amino-5-(trifluoromethyl)pyridin-3-yl]-N-[2-(pyridin-3-yl)propan-2-yl]-6,7-dihydrospiro[pyrazolo[5,1-c][1,4]oxazine-4,3′-pyrrolidine]-1′-carboxamide
(3R)-2′-[6-amino-5-(trifluoromethyl)pyridin-3-yl]-N-[2-(pyridin-4-yl)propan-2-yl]-5′,6′-dihydrospiro[pyrrolidine-3,4′-pyrrolo[1,2-b]pyrazole]-1-carboxamide
(3′R)-2-[6-amino-5-(trifluoromethyl)pyridin-3-yl]-N-[2-(pyridin-4-yl)propan-2-yl]-6,7-dihydrospiro[pyrazolo[5,1-c][1,4]oxazine-4,3′-pyrrolidine]-1′-carboxamide
(3R)-2′-[6-amino-5-(trifluoromethyl)pyridin-3-yl]-N-[2-(pyridin-3-yl)propan-2-yl]-5′,6′-dihydrospiro[pyrrolidine-3,4′-pyrrolo[1,2-b]pyrazole]-1-carboxamide
(3R)-2′-[6-amino-5-(difluoromethoxy)pyridin-3-yl]-N-[(1R)-1-phenylethyl]-5′,6′-dihydrospiro[pyrrolidine-3,4′-pyrrolo[1,2-b]pyrazole]-1-carboxamide
(3′R)-2-[6-amino-5-(difluoromethoxy)pyridin-3-yl]-N-[(1R)-1-phenylethyl]-6,7-dihydrospiro[pyrazolo[5,1-c][1,4]oxazine-4,3′-pyrrolidine]-1′-carboxamide
(3R)-2′-[6-amino-5-(trifluoromethyl)pyridin-3-yl]-N-[2-(pyridin-2-yl)propan-2-yl]-5′,6′-dihydrospiro[pyrrolidine-3,4′-pyrrolo[1,2-b]pyrazole]-1-carboxamide
2′-[6-amino-5-(trifluoromethyl) pyridin-3-yl]-N-[(1R)-1-phenylethyl]-5′,6′-dihydrospiro[pyrrolidine-3,4′-pyrrolo[1,2-b]pyrazole]-1-carboxamide (stereoisomer 1)
2′-[6-amino-5-(trifluoromethyl)pyridin-3-yl]-N-[(1R)-1-phenylethyl]-5′,6′-dihydrospiro[pyrrolidine-3,4′-pyrrolo[1,2-b]pyrazole]-1-carboxamide (stereoisomer 2)
(rac)-2′-[6-amino-5-(trifluoromethyl)pyridin-3-yl]-N-[2-(3,5-difluorophenyl)propan-2-yl]-5′,6′-dihydrospiro[pyrrolidine-3,4′-pyrrolo[1,2-b]pyrazole]-1-carboxamide
(rac)-1-{2′-[6-amino-5-(trifluoromethyl) pyridin-3-yl]-5′,6′-dihydrospiro[pyrrolidine-3,4′-pyrrolo[1,2-b]pyrazol]-1-yl}-2-(5-fluoropyridin-2-yl)-2-methylpropan-1-one
(rac)-2′-[6-amino-5-(trifluoromethyl)pyridin-3-yl]-N-[2-(6-methylpyridin-3-yl)propan-2-yl]-5′,6′-dihydrospiro[pyrrolidine-3,4′-pyrrolo[1,2-b]pyrazole]-1-carboxamide
(rac)-2′-[6-amino-5-(trifluoromethyl) pyridin-3-yl]-N-(1-phenylcyclohexyl)-5′,6′-dihydrospiro[pyrrolidine 3,4′-pyrrolo[1,2-b]pyrazole]-1-carboxamide
(rac)-2′-[6-amino-5-(trifluoromethyl)pyridin-3-yl]-N-[2-(5-fluoropyridin-2-yl)propan-2-yl]-5′,6′-dihydrospiro[pyrrolidine-3,4′-pyrrolo[1,2-b]pyrazole]-1-carboxamide
2′-[6-amino-5-(trifluoromethyl) pyridin-3-yl]-N-[(1R)-2,2,2-trifluoro-1-methoxy-1-phenylethyl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[2-(pyrimidin-4-yl)propan-2-yl]-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-[2-(pyrimidin-5-yl)propan-2-yl]-5′,6′-dihydrospiro[pyrrolidine-3,4′-pyrrolo[1,2-b]pyrazole]-1-carboxamide (rac)-{2′-[6-amino-5-(trifluoromethyl)pyridin-3-yl]-5′,6′-dihydrospiro[pyrrolidine-3,4′-pyrrolo[1,2-b]pyrazol]-1-yl}(3-phenyloxetan-3-yl)methanone
(rac)-1-{2′-[6-amino-5-(trifluoromethyl) pyridin-3-yl]-5′,6′-dihydrospiro[pyrrolidine-3,4′-pyrrolo[1,2-b]pyrazol]-1-yl}-2-(3,5-difluorophenyl)-2-methylpropan-1-one
(rac)-1-{2′-[6-amino-5-(trifluoromethyl) pyridin-3-yl]-5′,6′-dihydrospiro[pyrrolidine-3,4′-pyrrolo[1,2-b]pyrazol]-1-yl}-2-methyl-2-(6-methylpyridin-3-yl)propan-1-one
(rac)-2′-[6-amino-5-(trifluoromethyl)pyridin-3-yl]-N-(3-phenyloxetan-3-yl)-5′,6′-dihydrospiro[pyrrolidine-3,4′-pyrrolo[1,2-b]pyrazole]-1-carboxamide
(rac)-2′-[6-amino-5-(trifluoromethyl)pyridin-3-yl]-N-(propan-2-yl)-5′,6′-dihydrospiro[pyrrolidine-3,4′-pyrrolo[1,2-b]pyrazole]-1-carboxamide
(rac)-2′-[6-amino-5-(trifluoromethyl) pyridin-3-yl]-N-cyclobutyl-5′,6′-dihydrospiro[pyrrolidine-3,4′-pyrrolo[1,2-b]pyrazole]-1-carboxamide
(3R)-2′-[6-amino-5-(trifluoromethyl)pyridin-3-yl]-N-ethyl-5′,6′-dihydrospiro[pyrrolidine-3,4′-pyrrolo[1,2-b]pyrazole]-1-carboxamide
(3S)-2′-[6-amino-5-(trifluoromethyl)pyridin-3-yl]-N-ethyl-5′,6′-dihydrospiro[pyrrolidine-3,4′-pyrrolo[1,2-b]pyrazole]-1-carboxamide
(3R)-2′-[6-amino-5-(trifluoromethyl)pyridin-3-yl]-N-(propan-2-yl)-5′,6′-dihydrospiro[pyrrolidine-3,4′-pyrrolo[1,2-b]pyrazole]-1-carboxamide
(3S)-2′-[6-amino-5-(trifluoromethyl)pyridin-3-yl]-N-(propan-2-yl)-5′,6′-dihydrospiro[pyrrolidine-3,4′-pyrrolo[1,2-b]pyrazole]-1-carboxamide
(rac)-2-[6-amino-5-(trifluoromethyl)pyridin-3-yl]-N-cyclobutyl-6,7-dihydrospiro[pyrazolo[5,1-c][1,4]oxazine-4,3′-pyrrolidine]-1′-carboxamide
(rac)-2-[6-amino-5-(trifluoromethyl)pyridin-3-yl]-N-ethyl-6,7-dihydrospiro[pyrazolo[5,1-c][1,4]oxazine-4,3′-pyrrolidine]-1′-carboxamide
(rac)-2-[6-amino-5-(trifluoromethyl)pyridin-3-yl]-N-(propan-2-yl)-6,7-dihydrospiro[pyrazolo[5,1-c][1,4]oxazine-4,3′-pyrrolidine]-1′-carboxamide
2′-[6-amino-5-(trifluoromethyl)pyridin-3-yl]-N-cyclobutyl-5′,6′-dihydrospiro[azetidine-3,4′-pyrrolo[1,2-b]pyrazole]-1-carboxamide
2′-[6-amino-5-(trifluoromethyl)pyridin-3-yl]-N-ethyl-5′,6′-dihydrospiro[azetidine-3,4′-pyrrolo[1,2-b]pyrazole]-1-carboxamide
2′-[6-amino-5-(trifluoromethyl) pyridin-3-yl]-N-(propan-2-yl)-5′,6′-dihydrospiro[azetidine-3,4′-pyrrolo[1,2-b]pyrazole]-1-carboxamide
(rac)-2′-(6-amino-5-cyanopyridin-3-yl)-N-[2-(3-chloropyridin-4-yl)propan-2-yl]-5′,6′-dihydrospiro[pyrrolidine-3,4′-pyrrolo[1,2-b]pyrazole]-1-carboxamide
(rac)-2′-{6-amino-5-[(2-chlorophenyl)methoxy]pyridin-3-yl}-N-ethyl-5′,6′-dihydrospiro[pyrrolidine-3,4′-pyrrolo[1,2-b]pyrazole]-1-carboxamide
(rac)-2′-{6-amino-5-[(2-chlorophenyl)methoxy]pyridin-3-yl}-N-(propan-2-yl)-5′,6′-dihydrospiro[pyrrolidine-3,4′-pyrrolo[1,2-b]pyrazole]-1-carboxamide
(rac)-2′-{6-amino-5-[(2-chlorophenyl)methoxy]pyridin-3-yl}-N-cyclobutyl-5′,6′-dihydrospiro[pyrrolidine-3,4′-pyrrolo[1,2-b]pyrazole]-1-carboxamide
(rac)-2′-{6-amino-5-[(2-fluorophenyl)methoxy]pyridin-3-yl}-N-ethyl-5′,6′-dihydrospiro[pyrrolidine-3,4′-pyrrolo[1,2-b]pyrazole]-1-carboxamide
(rac)-2′-{6-amino-5-[(2-fluorophenyl)methoxy]pyridin-3-yl}-N-(propan-2-yl)-5′,6′-dihydrospiro[pyrrolidine-3,4′-pyrrolo[1,2-b]pyrazole]-1-carboxamide
(rac)-2′-{6-amino-5-[(2-fluorophenyl)methoxy]pyridin-3-yl}-N-cyclobutyl-5′,6′-dihydrospiro[pyrrolidine-3,4′-pyrrolo[1,2-b]pyrazole]-1-carboxamide
(rac)-2′-{6-amino-5-[(2-fluorophenyl)methoxy]pyridin-3-yl}-N-benzyl-5′,6′-dihydrospiro[pyrrolidine-3,4′-pyrrolo[1,2-b]pyrazole]-1-carboxamide
(rac)-2′-{6-amino-5-[(3-fluorophenyl)methoxy]pyridin-3-yl}-N-ethyl-5′,6′-dihydrospiro[pyrrolidine-3,4′-pyrrolo[1,2-b]pyrazole]-1-carboxamide
(rac)-2′-{6-amino-5-[(3-fluorophenyl)methoxy]pyridin-3-yl}-N-(propan-2-yl)-5′,6′-dihydrospiro[pyrrolidine-3,4′-pyrrolo[1,2-b]pyrazole]-1-carboxamide
(rac)-2′-{6-amino-5-[(3-fluorophenyl)methoxy]pyridin-3-yl}-N-cyclobutyl-5′,6′-dihydrospiro[pyrrolidine-3,4′-pyrrolo[1,2-b]pyrazole]-1-carboxamide
(rac)-2′-{6-amino-5-[(3-fluorophenyl)methoxy]pyridin-3-yl}-N-benzyl-5′,6′-dihydrospiro[pyrrolidine-3,4′-pyrrolo[1,2-b]pyrazole]-1-carboxamide
(rac)-2′-{6-amino-5-[(pyridin-2-yl)methoxy]pyridin-3-yl}-N-ethyl-5′,6′-dihydrospiro[pyrrolidine-3,4′-pyrrolo[1,2-b]pyrazole]-1-carboxamide
(rac)-2′-{6-amino-5-[(pyridin-2-yl)methoxy]pyridin-3-yl}-N-(propan-2-yl)-5′,6′-dihydrospiro[pyrrolidin 3,4′-pyrrolo[1,2-b]pyrazole]-1-carboxamide
(rac)-2′-{6-amino-5-[(pyridin-2-yl)methoxy]pyridin-3-yl}-N-cyclobutyl-5′,6′-dihydrospiro[pyrrolidine-3,4′-pyrrolo[1,2-b]pyrazole]-1-carboxamide
(rac)-2′-{6-amino-5-[(2-chloro-5-fluorophenyl)methoxy]pyridin-3-yl}-N-ethyl-5′,6′-dihydrospiro[pyrrolidine-3,4′-pyrrolo[1,2-b]pyrazole]-1-carboxamide
(rac)-2′-{6-amino-5-[(2-chloro-5-fluorophenyl)methoxy]pyridin-3-yl}-N-(propan-2-yl)-5′,6′-dihydrospiro[pyrrolidine-3,4′-pyrrolo[1,2-b]pyrazole]-1-carboxamide
2′-{6-amino-5-[(1R)-1-(pyridin-2-yl)ethoxy]pyridin-3-yl}-N-(propan-2-yl)-5′,6′-dihydrospiro[pyrrolidin-3,4′-pyrrolo[1,2-b]pyrazole]-1-carboxamide
2′-{6-amino-5-[(1R)-1-(pyridin-2-yl)ethoxy]pyridin-3-yl}-N-ethyl-5′,6′-dihydrospiro[pyrrolidine-3,4′-pyrrolo[1,2-b]pyrazole]-1-carboxamide
2′-{6-amino-5-[(1S)-1-(pyridin-2-yl)ethoxy]pyridin-3-yl}-N-(propan-2-yl)-5′,6′-dihydrospiro[pyrrolidine-3,4′-pyrrolo[1,2-b]pyrazole]-1-carboxamide
2′-{6-amino-5-[(1S)-1-(pyridin-2-yl)ethoxy]pyridin-3-yl}-N-ethyl-5′,6′-dihydrospiro[pyrrolidine-3,4′-pyrrolo[1,2-b]pyrazole]-1-carboxamide
2′-(6-amino-5-{[1-(5-chloro-3-fluoropyridin-2-yl)ethyl]oxy}pyridin-3-yl)-N-ethyl-5′,6′-dihydrospiro[pyrrolidine-3,4′-pyrrolo[1,2-b]pyrazole]-1-carboxamide (diastereomeric mixture)
(rac)-2′-[6-amino-5-(benzyloxy)pyridin-3-yl]-N-cyclobutyl-5′,6′-dihydrospiro[pyrrolidine-3,4′-pyrrolo[1,2-b]pyrazole]-1-carboxamide
2′-{6-amino-5-[(pyridazin-3-yl)methoxy]pyridin-3-yl}-N-ethyl-5′,6′-dihydrospiro[pyrrolidine-3,4′-pyrrolo[1,2-b]pyrazole]-1-carboxamide (stereoisomer 2)
2′-{6-amino-5-[(pyridazin-3-yl)methoxy]pyridin-3-yl}-N-ethyl-5′,6′-dihydrospiro[pyrrolidine-3,4′-pyrrolo[1,2-b]pyrazole]-1-carboxamide (stereoisomer 1)
(3S)-2′-{6-amino-5-[1-(pyrimidin-5-yl)ethoxy]pyridin-3-yl}-N-ethyl-5′,6′-dihydro-1H-spiro[pyrrolidine-3,4′-pyrrolo[1,2-b]pyrazole]-1-carboxamide (isomer 1)
(3S)-2′-{6-amino-5-[1-(pyrimidin-5-yl)ethoxy]pyridin-3-yl}-N-ethyl-5′,6′-dihydro-1H-spiro[pyrrolidine-3,4′-pyrrolo[1,2-b]pyrazole]-1-carboxamide (isomer 2)
(rac)-2′-(2-amino-1′-methyl-1′,2′,3′,6′-tetrahydro[3,4′-bipyridin]-5-yl)-N-ethyl-5′,6′-dihydrospiro[pyrrolidine-3,4′-pyrrolo[1,2-b]pyrazole]-1-carboxamide
2′-{6-amino-5-[(1S)-1-(pyridin-2-yl)ethoxy]pyridin-3-yl}-N-ethyl-5′,6′-dihydrospiro[azetidine-3,4′-pyrrolo[1,2-b]pyrazole]-1-carboxamide
2′-{6-amino-5-[(1S)-1-(pyridin-2-yl)ethoxy]pyridin-3-yl}-N-(propan-2-yl)-5′,6′-dihydrospiro[azetidine-3,4′-pyrrolo[1,2-b]pyrazole]-1-carboxamide
2′-{6-amino-5-[(1S)-1-phenylethoxy]pyridin-3-yl}-N-ethyl-5′,6′-dihydrospiro[azetidine-3,4′-pyrrolo[1,2-b]pyrazole]-1-carboxamide
2′-{6-amino-5-[(1R)-1-phenylethoxy]pyridin-3-yl}-N-ethyl-5′,6′-dihydrospiro[azetidine-3,4′-pyrrolo[1,2-b]pyrazole]-1-carboxamide
(rac)-2-[6-amino-5-(trifluoromethyl)pyridin-3-yl]-N-[2-(3-chloropyridin-4-yl)propan-2-yl]-6,7-dihydrospiro[pyrazolo[5,1-c][1,4]oxazine-4,3′-pyrrolidine]-1′-carboxamide
(rac)-2′-[6-amino-5-(trifluoromethyl)pyridin-3-yl]-N-ethyl-5′,6′-dihydrospiro[pyrrolidine-3,4′-pyrrolo[1,2-b]pyrazole]-1-carboxamide
(rac)-2-[6-amino-5-(trifluoromethyl)pyridin-3-yl]-N-ethyl-6,7-dihydro-5H-spiro[pyrazolo[1,5-a]pyridine-4,3′-pyrrolidine]-1′-carboxamide
(rac)-2′-(6-amino-5-cyanopyridin-3-yl)-N-ethyl-5′,6′-dihydrospiro[pyrrolidine-3,4′-pyrrolo[1,2-b]pyrazole]-1-carboxamide
(rac)-2′-[6-amino-5-(trifluoromethoxy)pyridin-3-yl]-N-ethyl-5′,6′-dihydrospiro[pyrrolidine-3,4′-pyrrolo[1,2-b]pyrazole]-1-carboxamide
(rac)-2′-[6-amino-5-(difluoromethoxy)pyridin-3-yl]-N-ethyl-5′,6′-dihydrospiro[pyrrolidine-3,4′-pyrrolo[1,2-b]pyrazole]-1-carboxamide
(rac)-2′-[6-amino-5-(trifluoroacetyl)pyridin-3-yl]-N-ethyl-5′,6′-dihydrospiro[pyrrolidine-3,4′-pyrrolo[1,2-b]pyrazole]-1-carboxamide
2′-(6-amino-5-{[1-phenylethyl]sulfanyl}pyridin-3-yl)-N-ethyl-5′,6′-dihydrospiro[pyrrolidine-3,4′-pyrrolo[1,2-b]pyrazole]-1-carboxamide (diastereomeric mixture)
(rac)-2′-{6-amino-5-[(cyclohexylmethyl)sulfanyl]pyridin-3-yl}-N-ethyl-5′,6′-dihydrospiro[pyrrolidine-3,4′-pyrrolo[1,2-b]pyrazole]-1-carboxamide
(rac)-2′-[6-amino-5-(benzylsulfanyl)pyridin-3-yl]-N-ethyl-5′,6′-dihydrospiro[pyrrolidine-3,4′-pyrrolo[1,2-b]pyrazole]-1-carboxamide
(rac)-2′-[6-amino-5-(2-hydroxypropan-2-yl)pyridin-3-yl]-N-ethyl-5′,6′-dihydrospiro[pyrrolidine-3,4′-pyrrolo[1,2-b]pyrazole]-1-carboxamide
2′-(6-amino-5-{[1-cyclohexylethyl]sulfanyl}pyridin-3-yl)-N-ethyl-5′,6′-dihydrospiro[pyrrolidine-3,4′-pyrrolo[1,2-b]pyrazole]-1-carboxamide (diastereomeric mixture)
2′-{6-amino-5-[(1R)-1-(pyridin-3-yl)ethoxy]pyridin-3-yl}-N-ethyl-5′,6′-dihydrospiro[pyrrolidine-3,4′-pyrrolo[1,2-b]pyrazole]-1-carboxamide
2′-{6-amino-5-[(1S)-1-(pyridin-3-yl)ethoxy]pyridin-3-yl}-N-ethyl-5′,6′-dihydrospiro[pyrrolidine-3,4′-pyrrolo[1,2-b]pyrazole]-1-carboxamide
(rac)-2′-{6-amino-5-[(cyclopentylmethyl)sulfanyl]pyridin-3-yl}-N-ethyl-5′,6′-dihydrospiro[pyrrolidine-3,4′-pyrrolo[1,2-b]pyrazole]-1-carboxamide
(rac)-2′-(6-amino-5-nitropyridin-3-yl)-N-ethyl-5′,6′-dihydrospiro[pyrrolidine-3,4′-pyrrolo[1,2-b]pyrazole]-1-carboxamide
(rac)-2′-[6-amino-5-(benzyloxy)pyridin-3-yl]-N-ethyl-5′,6′-dihydrospiro[pyrrolidine-3,4′-pyrrolo[1,2-b]pyrazole]-1-carboxamide
(rac)-2′-(6-amino-5-methylpyridin-3-yl)-N-ethyl-5′,6′-dihydrospiro[pyrrolidine-3,4′-pyrrolo[1,2-b]pyrazole]-1-carboxamide
(rac)-2-(6-amino-5-nitropyridin-3-yl)-N-ethyl-6,7-dihydro-5H-spiro[pyrazolo[1,5-a]pyridine-4,3′-pyrrolidine]-1′-carboxamide
methyl 2-amino-5-[(rac)-1-(ethylcarbamoyl)-5′,6′-dihydrospiro[pyrrolidine-3,4′-pyrrolo[1,2-b]pyrazol]-2′-yl]pyridine-3-carboxylate
(rac)-2′-(6-amino-5-phenylpyridin-3-yl)-N-ethyl-5′,6′-dihydrospiro[pyrrolidine-3,4′-pyrrolo[1,2-b]pyrazole]-1-carboxamide
(rac)-2′-[6-amino-5-(methylsulfanyl)pyridin-3-yl]-N-ethyl-5′,6′-dihydrospiro[pyrrolidine-3,4′-pyrrolo[1,2-b]pyrazole]-1-carboxamide
(rac)-2′-[6-amino-5-(4-methylphenyl)pyridin-3-yl]-N-ethyl-5′,6′-dihydrospiro[pyrrolidine-3,4′-pyrrolo[1,2-b]pyrazole]-1-carboxamide
(rac)-2′-[6-amino-5-(2-methylphenyl)pyridin-3-yl]-N-ethyl-5′,6′-dihydrospiro[pyrrolidine-3,4′-pyrrolo[1,2-b]pyrazole]-1-carboxamide
(rac)-2′-[6-amino-5-(3-methyl phenyl)pyridin-3-yl]-N-ethyl-5′,6′-dihydrospiro[pyrrolidine-3,4′-pyrrolo[1,2-b]pyrazole]-1-carboxamide
(rac)-2′-(6-amino-5-methoxypyridin-3-yl)-N-ethyl-5′,6′-dihydrospiro[pyrrolidine-3,4′-pyrrolo[1,2-b]pyrazole]-1-carboxamide
(rac)-2′-[6-amino-5-(3-fluorophenyl)pyridin-3-yl]-N-ethyl-5′,6′-dihydrospiro[pyrrolidine-3,4′-pyrrolo[1,2-b]pyrazole]-1-carboxamide
(rac)-2′-(2-amino[3,3′-bipyridin]-5-yl)-N-ethyl-5′,6′-dihydrospiro[pyrrolidine-3,4′-pyrrolo[1,2-b]pyrazole]-1-carboxamide
(rac)-2′-[6-amino-5-(2-fluorophenyl)pyridin-3-yl]-N-ethyl-5′,6′-dihydrospiro[pyrrolidine-3,4′-pyrrolo[1,2-b]pyrazole]-1-carboxamide
2′-{6-amino-5-[(1R)-1-(2,6-dichloro-3-fluorophenyl)ethoxy]pyridin-3-yl}-N-ethyl-5′,6′-dihydrospiro[pyrrolidine-3,4′-pyrrolo[1,2-b]pyrazole]-1-carboxamide (diastereomeric mixture)
(rac)-2-[6-amino-5-(2-fluorophenyl)pyridin-3-yl]-N-ethyl-6,7-dihydro-5H-spiro[pyrazolo[1,5-a]pyridine-4,3′-pyrrolidine]-1′-carboxamide
(rac)-2′-[6-amino-5-(2-methoxyphenyl)pyridin-3-yl]-N-ethyl-5′,6′-dihydrospiro[pyrrolidine-3,4′-pyrrolo[1,2-b]pyrazole]-1-carboxamide
(rac)-2′-(2-amino[3,4′-bipyridin]-5-yl)-N-ethyl-5′,6′-dihydrospiro[pyrrolidine-3,4′-pyrrolo[1,2-b]pyrazole]-1-carboxamide
(rac)-2′-{6-amino-5-[3-(methanesulfonyl)phenyl]pyridin-3-yl}-N-ethyl-5′,6′-dihydrospiro[pyrrolidine-3,4′-pyrrolo[1,2-b]pyrazole]-1-carboxamide
(rac)-2′-[6-amino-5-(phenylsulfanyl)pyridin-3-yl]-N-ethyl-5′,6′-dihydrospiro[pyrrolidine-3,4′-pyrrolo[1,2-b]pyrazole]-1-carboxamide
(rac)-2′-{6-amino-5-[4-(methanesulfonyl)phenyl]pyridin-3-yl}-N-ethyl-5′,6′-dihydrospiro[pyrrolidine-3,4′-pyrrolo[1,2-b]pyrazole]-1-carboxamide
(rac)-2′-[5-(4-acetylphenyl)-6-aminopyridin-3-yl]-N-ethyl-5′,6′-dihydrospiro[pyrrolidine-3,4′-pyrrolo[1 b]pyrazole]-1-carboxamide
(rac)-2′-{6-amino-5-[4-(methanesulfinyl)phenyl]pyridin-3-yl}-N-ethyl-5′,6′-dihydrospiro[pyrrolidine-3,4′-pyrrolo[1,2-b]pyrazole]-1-carboxamide
(rac)-2′-[6-amino-5-(3-methoxyphenyl)pyridin-3-yl]-N-ethyl-5′,6′-dihydrospiro[pyrrolidine-3,4′-pyrrolo[1,2-b]pyrazole]-1-carboxamide
2′-{6-amino-5-[(1R)-1-(2,6-dichloro-3-fluorophenyl)ethoxy]pyridin-3-yl}-N-ethyl-5′,6′-dihydrospiro[azetidine-3,4′-pyrrolo[1,2-b]pyrazole]-1-carboxamide
(rac)-2′-{6-amino-5-[4-(cyclopropanesulfonyl)phenyl]pyridin-3-yl}-N-ethyl-5′,6′-dihydrospiro[pyrrolidine-3,4′-pyrrolo[1,2-b]pyrazole]-1-carboxamide
(rac)-2′-[6-amino-5-(4-sulfamoylphenyl)pyridin-3-yl]-N-ethyl-5′,6′-dihydrospiro[pyrrolidine-3,4′-pyrrolo[1,2-b]pyrazole]-1-carboxamide
(rac)-2′-{6-amino-5-[4-(methylsulfamoyl)phenyl]pyridin-3-yl}-N-ethyl-5′,6′-dihydrospiro[pyrrolidine-3,4′-pyrrolo[1,2-b]pyrazole]-1-carboxamide
(rac)-2′-{6-amino-5-[4-(dimethyl sulfamoyl)phenyl]pyridin-3-yl}-N-ethyl-5′,6′-dihydrospiro[pyrrolidine-3,4′-pyrrolo[1,2-b]pyrazole]-1-carboxamide
(rac)-2′-[6-amino-5-(cyclohexylmethoxy)pyridin-3-yl]-N-ethyl-5′,6′-dihydrospiro[pyrrolidine-3,4′-pyrrolo[1,2-b]pyrazole]-1-carboxamide
(rac)-2′-{6-amino-5-[(pyridin-3-yl)methoxy]pyridin-3-yl}-N-ethyl-5′,6′-dihydrospiro[pyrrolidine-3,4′-pyrrolo[1,2-b]pyrazole]-1-carboxamide
(rac)-2′-{6-amino-5-[(4-fluorophenyl)methoxy]pyridin-3-yl}-N-ethyl-5′,6′-dihydrospiro[pyrrolidine-3,4′-pyrrolo[1,2-b]pyrazole]-1-carboxamide
2′-(6-amino-5-{[1-phenylethyl]oxy}pyridin-3-yl)-N-ethyl-5′,6′-dihydrospiro[pyrrolidine-3,4′-pyrrolo[1,2-b]pyrazole]-1-carboxamide (diastereomeric mixture)
2-{6-amino-5-[(1R)-1-(2,6-dichloro-3-fluorophenyl)ethoxy]pyridin-3-yl}-N-ethyl-6,7-dihydrospiro[pyrazolo[5,1-c][1,4]oxazine-4,3′-pyrrolidine]-1′-carboxamide
(rac)-2′-[6-amino-5-(propan-2-yl)pyridin-3-yl]-N-ethyl-5′,6′-dihydrospiro[pyrrolidine-3,4′-pyrrolo[1,2-b]pyrazole]-1-carboxamide
(rac)-2-[6-amino-5-(methylcarbamoyl)pyridin-3-yl]-N-ethyl-6,7-dihydrospiro[pyrazolo[5,1-c][1,4]oxazine-4,3′-pyrrolidine]-1′-carboxamide
(rac)-2′-[6-amino-5-(methylcarbamoyl)pyridin-3-yl]-N-ethyl-5′,6′-dihydrospiro[pyrrolidine-3,4′-pyrrolo[1,2-b]pyrazole]-1-carboxamide
(rac)-2′-(6-amino-5-cyclopropylpyridin-3-yl)-N-ethyl-5′,6′-dihydrospiro[pyrrolidine-3,4′-pyrrolo[1,2-b]pyrazole]-1-carboxamide
(rac)-2′-[6-amino-5-(methylcarbamoyl)pyridin-3-yl]-N-(1-phenylcyclobutyl)-5′,6′-dihydrospiro[pyrrolidine-3,4′-pyrrolo[1,2-b]pyrazole]-1-carboxamide
(rac)-2′-[6-amino-5-(dimethylcarbamoyl)pyridin-3-yl]-N-(1-phenylcyclobutyl)-5′,6′-dihydrospiro[pyrrolidine-3,4′-pyrrolo[1,2-b]pyrazole]-1-carboxamide
2′-{6-amino-5-[(1R)-1-(pyridin-4-yl)ethoxy]pyridin-3-yl}-N-ethyl-5′,6′-dihydrospiro[pyrrolidine-3,4′-pyrrolo[1,2-b]pyrazole]-1-carboxamide
2′-{6-amino-5-[(1S)-1-(pyridin-4-yl)ethoxy]pyridin-3-yl}-N-ethyl-5′,6′-dihydrospiro[pyrrolidine-3,4′-pyrrolo[1,2-b]pyrazole]-1-carboxamide
(rac)-2′-[6-amino-5-(1-methyl-1H-pyrazol-3-yl)pyridin-3-yl]-N-ethyl-5′,6′-dihydrospiro[pyrrolidine-3,4′-pyrrolo[1,2-b]pyrazole]-1-carboxamide
(rac)-2′-[6-amino-5-(1,2-thiazol-5-yl)pyridin-3-yl]-N-ethyl-5′,6′-dihydrospiro[pyrrolidine-3,4′-pyrrolo[1,2-b]pyrazole]-1-carboxamide
(rac)-2′-[6-amino-5-(1,5-dimethyl-1H-pyrazol-4-yl)pyridin-3-yl]-N-ethyl-5′,6′-dihydrospiro[pyrrolidine-3,4′-pyrrolo[1,2-b]pyrazole]-1-carboxamide
(rac)-2′-{6-amino-5-[(trifluoromethyl)sulfanyl]pyridin-3-yl}-N-ethyl-5′,6′-dihydrospiro[pyrrolidine-3,4′-pyrrolo[1,2-b]pyrazole]-1-carboxamide
5-[1-methyl-5′,6′-dihydrospiro[pyrrolidine-3,4′-pyrrolo[1,2-b]pyrazol]-2′-yl]-3-(trifluoromethyl)pyridin-2-amine (stereoisomer 1)
5-[1-methyl-5′,6′-dihydrospiro[pyrrolidine-3,4′-pyrrolo[1,2-b]pyrazol]-2′-yl]-3-(trifluoromethyl) pyridin-2-amine (stereoisomer 2)
(3R)-2′-{6-amino-5-[(1R)-1-(2-chlorophenyl)ethoxy]pyridin-3-yl}-N-ethyl-5′,6′-dihydrospiro[pyrrolidine-3,4′-pyrrolo[1,2-b]pyrazole]-1-carboxamide
2′-{6-amino-5-[(1R)-1-(2-chlorophenyl)ethoxy]pyridin-3-yl}-N-ethyl-5′,6′-dihydrospiro[azetidine-3,4′-pyrrolo[1,2-b]pyrazole]-1-carboxamide
2′-{6-amino-5-[(1S)-1-(2-chlorophenyl)ethoxy]pyridin-3-yl}-N-ethyl-5′,6′-dihydrospiro[azetidine-3,4′-pyrrolo[1,2-b]pyrazole]-1-carboxamide
(3′R)-2-{6-amino-5-[(1R)-1-(2,6-dichloro-3-fluorophenyl)ethoxy]pyridin-3-yl}-N-ethyl-6,7-dihydrospiro[pyrazolo[5,1-c][1,4]oxazine-4,3′-pyrrolidine]-1′-carboxamide
(3R)-2′-{6-amino-5-[(1R)-1-(pyridin-2-yl)ethoxy]pyridin-3-yl}-N-ethyl-5′,6′-dihydrospiro[pyrrolidine-3,4′-pyrrolo[1,2-b]pyrazole]-1-carboxamide
2′-{6-amino-5-[(1R)-1-(pyridin-3-yl)ethoxy]pyridin-3-yl}-N-ethyl-5′,6′-dihydrospiro[azetidine-3,4′-pyrrolo[1,2-b]pyrazole]-1-carboxamide
(3R)-2′-{6-amino-5-[1-(pyrimidin-5-yl)ethoxy]pyridin-3-yl}-N-ethyl-5′,6′-dihydro-1H-spiro[pyrrolidine-3,4′-pyrrolo[1,2-b]pyrazole]-1-carboxamide (isomer 1)
((3R)-2′-{6-amino-5-[1-(pyrimidin-5-yl)ethoxy]pyridin-3-yl}-N-ethyl-5′,6′-dihydro-1H-spiro[pyrrolidine-3,4′-pyrrolo[1,2-b]pyrazole]-1-carboxamide (isomer 2)
(rac)-2′-[6-amino-5-(difluoromethyl)pyridin-3-yl]-N-ethyl-5′,6′-dihydrospiro[pyrrolidine-3,4′-pyrrolo[1,2-b]pyrazole]-1-carboxamide
2′-{6-amino-5-[(1S)-1-(2-chlorophenyl)ethoxy]pyridin-3-yl}-N-ethyl-5′,6′-dihydrospiro[pyrrolidine-3,4′-pyrrolo[1,2-b]pyrazole]-1-carboxamide
2′-{6-amino-5-[(1R)-1-(2-chlorophenyl)ethoxy]pyridin-3-yl}-N-ethyl-5′,6′-dihydrospiro[pyrrolidine-3,4′-pyrrolo[1,2-b]pyrazole]-1-carboxamide
(rac)-2′-{6-amino-5-[(3-methylphenyl)methoxy]pyridin-3-yl}-N-ethyl-5′,6′-dihydrospiro[pyrrolidine-3,4′-pyrrolo[1,2-b]pyrazole]-1-carboxamide
(rac)-2′-{6-amino-5-[(1-methyl-1H-pyrazol-5-yl)methoxy]pyridin-3-yl}-N-ethyl-5′,6′-dihydrospiro[pyrrolidine-3,4′-pyrrolo[1,2-b]pyrazole]-1-carboxamide
(rac)-2′-{6-amino-5-[(1-methyl-1H-pyrazol-4-yl)methoxy]pyridin-3-yl}-N-ethyl-5′,6′-dihydrospiro[pyrrolidine-3,4′-pyrrolo[1,2-b]pyrazole]-1-carboxamide
(rac)-2′-{6-amino-5-[(1,5-dimethyl-1H-pyrazol-3-yl)methoxy]pyridin-3-yl}-N-ethyl-5′,6′-dihydrospiro[pyrrolidine-3,4′-pyrrolo[1,2-b]pyrazole]-1-carboxamide
(rac)-2′-{6-amino-5-[(4-methyl phenyl)methoxy]pyridin-3-yl}-N-ethyl-5′,6′-dihydrospiro[pyrrolidine-3,4′-pyrrolo[1,2-b]pyrazole]-1-carboxamide
2′-{6-amino-5-[(1R)-1-(3-methylphenyl)ethoxy]pyridin-3-yl}-N-ethyl-5′,6′-dihydrospiro[pyrrolidine-3,4′-pyrrolo[1,2-b]pyrazole]-1-carboxamide
(rac)-2′-{6-amino-5-[(propan-2-yl)oxy]pyridin-3-yl}-N-ethyl-5′,6′-dihydrospiro[pyrrolidine-3,4′-pyrrolo[1,2-b]pyrazole]-1-carboxamide
(3R)-2′-{6-amino-5-[(1S)-1-(1-methyl-1H-pyrazol-3-yl)ethoxy]pyridin-3-yl}-N-ethyl-5′,6′-dihydrospiro[pyrrolidine-3,4′-pyrrolo[1,2-b]pyrazole]-1-carboxamide
(3R)-2′-{6-amino-5-[(1R)-1-(1-methyl-1H-pyrazol-3-yl)ethoxy]pyridin-3-yl}-N-ethyl-5′,6′-dihydrospiro[pyrrolidine-3,4′-pyrrolo[1,2-b]pyrazole]-1-carboxamide
(3R)-2′-(6-amino-5-{[1-(3,5-difluoropyridin-2-yl)ethyl]oxy}pyridin-3-yl)-N-ethyl-5′,6′-dihydrospiro[pyrrolidine-3,4′-pyrrolo[1,2-b]pyrazole]-1-carboxamide (stereoisomer 1)
(3R)-2′-(6-amino-5-{[1-(3,5-difluoropyridin-2-yl)ethyl]oxy}pyridin-3-yl)-N-ethyl-5′,6′-dihydrospiro[pyrrolidine-3,4′-pyrrolo[1,2-b]pyrazole]-1-carboxamide (stereoisomer 2)
(3R)-2′-{6-amino-5-[(1R)-1-(2-fluorophenyl)ethoxy]pyridin-3-yl}-N-ethyl-5′,6′-dihydrospiro[pyrrolidine 3,4′-pyrrolo[1,2-b]pyrazole]-1-carboxamide
(3R)-2′-{6-amino-5-[(1S)-1-(2-fluorophenyl)ethoxy]pyridin-3-yl}-N-ethyl-5′,6′-dihydrospiro[pyrrolidine-3,4′-pyrrolo[1,2-b]pyrazole]-1-carboxamide
(rac)-2′-(5-acetyl-6-aminopyridin-3-yl)-N-(1-phenylcyclobutyl)-5′,6′-dihydrospiro[pyrrolidine-3,4′-pyrrolo[1,2-b]pyrazole]-1-carboxamide
(3R)-2′-[6-amino-5-(trifluoromethoxy)pyridin-3-yl]-N-[2-(pyridin-2-yl)propan-2-yl]-5′,6′-dihydrospiro[pyrrolidine-3,4′-pyrrolo[1,2-b]pyrazole]-1-carboxamide
(3′R)-2-[6-amino-5-(trifluoromethyl)pyridin-3-yl]-N-[2-(pyridin-2-yl)propan-2-yl]-6,7-dihydrospiro[pyrazolo[5,1-c][1,4]oxazine-4,3′-pyrrolidine]-1′-carboxamide
(3R)-2′-(6-amino-5-cyanopyridin-3-yl)-N-[2-(pyridin-2-yl)propan-2-yl]-5′,6′-dihydrospiro[pyrrolidine-3,4′-pyrrolo[1,2-b]pyrazole]-1-carboxamide
(3′R)-2-(6-amino-5-cyanopyridin-3-yl)-N-[2-(pyridin-2-yl)propan-2-yl]-6,7-dihydrospiro[pyrazolo[5,1-c][1,4]oxazine-4,3′-pyrrolidine]-1′-carboxamide
(rac)-2′-[6-amino-5-(phenylcarbamoyl)pyridin-3-yl]-N-ethyl-5′,6′-dihydrospiro[pyrrolidine-3,4′-pyrrolo[1,2-b]pyrazole]-1-carboxamide
(rac)-2′-[6-amino-5-(cyclohexylcarbamoyl)pyridin-3-yl]-N-ethyl-5′,6′-dihydrospiro[pyrrolidine-3,4′-pyrrolo[1,2-b]pyrazole]-1-carboxamide
(rac)-2′-[6-amino-5-(diethylcarbamoyl)pyridin-3-yl]-N-ethyl-5′,6′-dihydrospiro[pyrrolidine-3,4′-pyrrolo[1,2-b]pyrazole]-1-carboxamide
(rac)-2′-(5-acetyl-6-aminopyridin-3-yl)-N-ethyl-5′,6′-dihydrospiro[pyrrolidine-3,4′-pyrrolo[1,2-b]pyrazole]-1-carboxamide
5-{1-[1-(1H-imidazol-2-yl)ethyl]-5′,6′-dihydrospiro[pyrrolidine-3,4′-pyrrolo[1,2-b]pyrazol]-2′-yl}-3-(trifluoromethyl)pyridin-2-amine
5-{1-[1-(1H-pyrazol-3-yl)ethyl]-5′,6′-dihydrospiro[pyrrolidine-3,4′-pyrrolo[1,2-b]pyrazol]-2′-yl}-3-(trifluoromethyl)pyridin-2-amine
5-{(3R)-1-[1-(1H-pyrazol-3-yl)ethyl]-5′,6′-dihydrospiro[pyrrolidine-3,4′-pyrrolo[1,2-b]pyrazol]-2′-yl}-3-(trifluoromethyl)pyridin-2-amine
5-{(3R)-1-[1-(1H-imidazol-2-yl)ethyl]-5′,6′-dihydrospiro[pyrrolidine-3,4′-pyrrolo[1,2-b]pyrazol]-2′-yl}-3-(trifluoromethyl)pyridin-2-amine
5-{(3R)-1-[1-(1H-imidazol-5-yl)ethyl]-5′,6′-dihydrospiro[pyrrolidine-3,4′-pyrrolo[1,2-b]pyrazol]-2′-yl}-3-(trifluoromethyl)pyridin-2-amine
5-{(3R)-1-[1-(1-methyl-1H-pyrazol-3-yl)ethyl]-5′,6′-dihydrospiro[pyrrolidine-3,4′-pyrrolo[1,2-b]pyrazol]-2′-yl}-3-(trifluoromethyl)pyridin in-2-amine
5-{(3R)-1-[1-(1H-pyrazol-4-yl)ethyl]-5′,6′-dihydrospiro[pyrrolidine-3,4′-pyrrolo[1,2-b]pyrazol]-2′-yl}-3-(trifluoromethyl)pyridin-2-amine
5-{(3R)-1-[1-(4-methyl-1H-imidazol-2-yl)ethyl]-5′,6′-dihydrospiro[pyrrolidine-3,4′-pyrrolo[1,2-b]pyrazol]-2′-yl}-3-(trifluoromethyl)pyridin-2-amine
5-{(3R)-1-[1-(3-methyl-1H-pyrazol-5′,6′-dihydrospiro[pyrrolidine-3,4′-pyrrolo[1,2-b]pyrazol]-]3-2′-yl}-3-(trifluoromethyl)pyridin-2-amine
5-{(3R)-1-[1-(4-methyl-1H-pyrazol-5-yl)ethyl]-5′,6′-dihydrospiro[pyrrolidine-3,4′-pyrrolo[1,2-b]pyrazol]-2′-yl}(trifluoromethyl)pyridin-2-amine
5-[(3S)-1-(pyridin-3-ylmethyl)-5′,6′-dihydrospiro[pyrrolidine-3,4′-pyrrolo[1,2-b]pyrazol]-2′-yl]-3-(trifluoromethyl)pyridin-2-amine
5-[(3R)-1-(pyridin-3-ylmethyl)-5′,6′-dihydrospiro[pyrrolidine-3,4′-pyrrolo[1,2-b]pyrazol]-2′-yl]-3-(trifluoromethyl)pyridin-2-amine
5-{(rac)-1-[(1H-pyrazol-3-yl)methyl]-5′,6′-dihydrospiro[pyrrolidine-3,4′-pyrrolo[1,2-b]pyrazol]-2′-yl}-3-(trifluoromethyl)pyridin-2-amine
5-[(3S)-1-(1H-imidazol-2-ylmethyl)-5′,6′-dihydrospiro[pyrrolidine-3,4′-pyrrolo[1,2-b]pyrazol]-2′-yl]-3-(trifluoromethyl)pyridin-2-amine
5-[(3R)-1-(1H-imidazol-2-ylmethyl)-5′,6′-dihydrospiro[pyrrolidine-3,4′-pyrrolo[1,2-b]pyrazol]-2′-yl]-3-(trifluoromethyl)pyridin-2-amine
5-{(rac)-1-[(4-methyl-1H-pyrazol-3-yl)]-5′,6′-dihydrospiro[pyrrolidine-3,4′-pyrrolo[1,2-b]pyrazol]-2′-yl}-3-(trifluoromethyl)pyridin-2-amine
5-{(rac)-1-[(1H-pyrazol-4-yl)methyl]-5′,6′-dihydrospiro[pyrrolidine-3,4′-pyrrolo[1,2-b]pyrazol]-2′-yl}-3-(trifluoromethyl)pyridin-2-amine
5-{(rac)-1-[(1-methyl-1H-pyrazol-4-yl)methyl]-5′,6′-dihydrospiro[pyrrolidine-3,4′-pyrrolo[1,2-b]pyrazol]-2′-yl}-3-(trifluoromethyl)pyridin in-2-amine
5-{(rac)-1-[(1H-imidazol-4-yl)methyl]3-5′,6′-dihydrospiro[pyrrolidine-3,4′-pyrrolo[1,2-b]pyrazol]-2′-yl}-3-(trifluoromethyl)pyridin-2-amine
5-{(rac)-1-[(1-methyl-1H-imidazol-4-yl)methyl]-5′,6′-dihydrospiro[pyrrolidine-3,4′-pyrrolo[1,2-b]pyrazol]-2′-yl}-3-(trifluoromethyl)pyridin-2-amine
5-{(rac)-1-[(4-chloro-1H-pyrazol-5-yl)methyl]-5′,6′-dihydrospiro[pyrrolidine-3,4′-pyrrolo[1,2-b]pyrazol]-2′-yl}-3-(trifluoromethyl)pyridin-2-amine
3-[(2-fluorophenyl)methoxy]-5-{(rac)-1-[(1H-imidazol-2-yl)methyl]-5′,6′-dihydrospiro[pyrrolidine-3,4′-pyrrolo[1,2-b]pyrazol]-2′-yl}pyridin-2-amine
3-[(3-fluorophenyl)methoxy]-5-{(rac)-1-[(1H-imidazol-2-yl)methyl]-5′,6′-dihydrospiro[pyrrolidine-3,4′-pyrrolo[1,2-b]pyrazol]-2′-yl}pyridin-2-amine
5-{(rac)-1-[(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine
5-{1-[(1H-imidazol-2-yl)methyl]-5′,6′-dihydrospiro[azetidine-3,4′-pyrrolo[1,2-b]pyrazol]-2′-yl}-3-(trifluoromethyl)pyridin-2-amine
5-{(rac)-1′-[(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine
2-amino-5-{1-[(1H-imidazol-2-yl)methyl]-5′,6′-dihydrospiro[azetidine-3,4′-pyrrolo[1,2-b]pyrazol]-2′-yl}pyridine-3-carbonitrile
5-{(rac)-1-[(1-methyl-1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine
5-{(rac)-1-[(1-methyl-1H-pyrazol-3-yl)methyl]-5′,6′-dihydrospiro[pyrrolidine-3,4′-pyrrolo[1,2-b]pyrazol]-2′-yl}-3-(trifluoromethyl)pyridin-2-amine
2-amino-5-{(rac)-1-[(1H-imidazol-2-yl)methyl]-5′,6′-dihydrospiro[pyrrolidine-3,4′-pyrrolo[1,2-b]pyrazol]-2′-yl}pyridine-3-carbonitrile
5-{1-[(1-methyl-1H-imidazol-2-yl)methyl]-5′,6′-dihydrospiro[azetidine-3,4′-pyrrolo[1,2-b]pyrazol]-2′-yl}-3-(trifluoromethyl)pyridin-2-amine
5-{(rac)-1-[(5-methyl-1H-pyrazol-3-yl)methyl]-5′,6′-dihydrospiro[pyrrolidine-3,4′-pyrrolo[1,2-b]pyrazol]-2′-yl}-3-(trifluoromethyl)pyridin-2-amine
5-{(rac)-1-[(2,4-dimethyl-1H-imidazol-5-yl)methyl]-5′,6′-dihydrospiro[pyrrolidine-3,4′-pyrrolo[1,2-b]pyrazol]-2′-yl}-3-(trifluoromethyl)pyridin-2-amine
5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine
5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (stereoisomer 1)
5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (stereoisomer 2)
5-{(3R)-1-[(1-(1H-imidazol-2-yl)propyl]-5′,6′-dihydrospiro[pyrrolidine-3,4′-pyrrolo[1,2-b]pyrazol]-2′-yl}-3-(trifluoromethyl)pyridin-2-amine
5-{(3R)-1-[1-(1H-imidazol-2-yl)propyl]-5′,6′-dihydrospiro[pyrrolidine-3,4′-pyrrolo[1,2-b]pyrazol]-2′-yl}-3-(trifluoromethyl)pyridin-2-amine (stereoisomer 1)
5-{(3R)-1-[1-(1H-imidazol-2-yl)propyl]-5′,6′-dihydrospiro[pyrrolidine-3,4′-pyrrolo[1,2-b]pyrazol]-2′-yl}-3-(trifluoromethyl)pyridin-2-amine (stereoisomer 2)
5-{(3R)-1-[1-(1-methyl-1H-imidazol-2-yl)ethyl]-5′,6′-dihydrospiro[pyrrolidine-3,4′-pyrrolo[1,2-b]pyrazol]-2′-yl}-3-(trifluoromethyl)pyridin-2-amine
3-(trifluoromethyl)-5-[(3R)-1-{[4-(trifluoromethyl)-1H-imidazol-2-yl]methyl}-5′,6′-dihydrospiro[pyrrolidine-3,4′-pyrrolo[1,2-b]pyrazol]-2′-yl]pyridin-2-amine
2-amino-5-[(rac)-1-(ethylcarbamoyl)-5′,6′-dihydrospiro[pyrrolidine-3,4′-pyrrolo[1,2-b]pyrazol]-2′-yl]pyridine-3-carboxylic acid
5-[(3S)-1-(pyridin-2-ylmethyl)-5′,6′-dihydrospiro[pyrrolidine-3,4′-pyrrolo[1,2-b]pyrazol]-2′-yl]-3-(trifluoromethyl)pyridin-2-amine
5-[(3R)-1-(pyridin-2-ylmethyl)-5′,6′-dihydrospiro[pyrrolidine-3,4′-pyrrolo[1,2-b]pyrazol]-2′-yl]-3-(trifluoromethyl)pyridin-2-amine
5-[(3S)-1-(pyridin-4-ylmethyl)-5′,6′-dihydrospiro[pyrrolidine-3,4′-pyrrolo[1,2-b]pyrazol]-2′-yl]-3-(trifluoromethyl)pyridin-2-amine
5-[(3R)-1-(pyridin-4-ylmethyl)-5′,6′-dihydrospiro[pyrrolidine-3,4′-pyrrolo[1,2-b]pyrazol]-2′-yl]-3-(trifluoromethyl)pyridin-2-amine
5-{(3R)-1-[3-(dimethyl amino)propyl]-5′,6′-dihydrospiro[pyrrolidine-3,4′-pyrrolo[1,2-b]pyrazol]-2′-yl}-3-(trifluoromethyl)pyridin-2-amine
5-{(3R)-1-[2-(dimethylamino)ethyl]-5′,6′-dihydrospiro[pyrrolidine-3,4′-pyrrolo[1,2-b]pyrazol]-2′-yl}-3-(trifluoromethyl)pyridin-2-amine
2-amino-1-{2′-[6-amino-5-(trifluoromethyl)pyridin-3-yl]-5′,6′-dihydrospiro[pyrrolidine-3,4′-pyrrolo[1,2-b]pyrazol]-1-yl}ethan-1-one (stereoisomer 2)
2-amino-1-{2′-[6-amino-5-(trifluoromethyl)pyridin-3-yl]-5′,6′-dihydrospiro[pyrrolidine-3,4′-pyrrolo[1,2-b]pyrazol]-1-yl}ethan-1-one (stereoisomer 1)
2-amino-1-{2-[6-amino-5-(trifluoromethyl)pyridin-3-yl]-6,7-dihydrospiro[pyrazolo[5,1-c][1,4]oxazine-4,3′-pyrrolidin]-1′-yl}ethan-1-one (stereoisomer 2)
2-amino-1-{2-[6-amino-5-(trifluoromethyl) pyridin-3-yl]-6,7-dihydrospiro[pyrazolo[5,1-c][1,4]oxazine-4,3′-pyrrolidin]-1′-yl}ethan-1-one (stereoisomer 1)
2-amino-1-{2′-[6-amino-5-(trifluoromethyl)pyridin-3-yl]-5′,6′-dihydrospiro[azetidine-3,4′-pyrrolo[1,2-b]pyrazol]-1-yl}ethan-1-one
(2S)-2-amino-1-{2′-[6-amino-5-(trifluoromethyl)pyridin-3-yl]-5′,6′-dihydrospiro[azetidine-3,4′-pyrrolo[1,2-b]pyrazol]-1-yl}propan-1-one
(2R)-2-amino-1-{2′-[6-amino-5-(trifluoromethyl)pyridin-3-yl]-5′,6′-dihydrospiro[azetidine-3,4′-pyrrolo[1,2-b]pyrazol]-1-yl}propan-1-one
(rac)-2′-[6-amino-5-(1-methylpiperidin-4-yl)pyridin-3-yl]-N-ethyl-5′,6′-dihydrospiro[pyrrolidine-3,4′-pyrrolo[1,2-b]pyrazole]-1-carboxamide
5-{(rac)-1-[(5-methyl-1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine
2′-{6-amino-5-[(1R)-1-(2,6-dichloro-3-fluorophenyl)ethoxy]pyridin-3-yl}-N-ethyl-5′,6′-dihydrospiro[pyrrolidine-3,4′-pyrrolo[1,2-b]pyrazole]-1-carboxamide (isomer 1)
2′-{6-amino-5-[(1R)-1-(2,6-dichloro-3-fluorophenyl)ethoxy]pyridin-3-yl}-N-ethyl-5′,6′-dihydrospiro[pyrrolidine-3,4′-pyrrolo[1,2-b]pyrazole]-1-carboxamide (isomer 2)
5-{(3′R)-1′-[(1S)-1-(1H-imidazol-2-yl)propyl]-6,7-dihydrospiro[pyrazolo[5,1-c][1,4]oxazine-4,3′-pyrrolidin]-2-yl}-3-(trifluoromethyl)pyridin-2-amine
5-{(3′R)-1′-[(1R)-1-(1H-imidazol-2-yl)propyl]-6,7-dihydrospiro[pyrazolo[5,1-c][1,4]oxazine-4,3′-pyrrolidin]-2-yl}-3-(trifluoromethyl)pyridin-2-amine
5-{(3′R)-1′-[(S)-cyclopropyl(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine
5-{(3′R)-1′-[(R)-cyclopropyl(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine
5-{(3R)-1-[(S)-cyclopropyl(1H-imidazol-2-yl)methyl]-5′,6′-dihydrospiro[pyrrolidine-3,4′-pyrrolo[1,2-b]pyrazol]-2′-yl}-3-(trifluoromethoxy)pyridin-2-amine
5-{(3R)-1-[(R)-cyclopropyl(1H-imidazol-2-yl)methyl]-5′,6′-dihydrospiro[pyrrolidine-3,4′-pyrrolo[1,2-b]pyrazol]-2′-yl}-3-(trifluoromethoxy)pyridin-2-amine
5-{(3R)-1-[(1S)-1-(1H-imidazol-2-yl)propyl]-5′,6′-dihydrospiro[pyrrolidine-3,4′-pyrrolo[1,2-b]pyrazol]-2′-yl}-3-(trifluoromethoxy)pyridin-2-amine
5-{(3R)-1-[(1R)-1-(1H-imidazol-2-yl)propyl]-5′,6′-dihydrospiro[pyrrolidine-3,4′-pyrrolo[1,2-b]pyrazol]-2′-yl}-3-(trifluoromethoxy)pyridin-2-amine
5-{(3R)-1-[(R)-cyclopropyl(1H-imidazol-2-yl)methyl]-5′,6′-dihydrospiro[pyrrolidine-3,4′-pyrrolo[1,2-b]pyrazol]-2′-yl}-3-(difluoromethoxy)pyridin-2-amine
5-{(3R)-1-[(S)-cyclopropyl(1H-imidazol-2-yl)methyl]-5′,6′-dihydrospiro[pyrrolidine-3,4′-pyrrolo[1,2-b]pyrazol]-2′-yl}-3-(difluoromethoxy)pyridin-2-amine
3-(difluoromethoxy)-5-{(3R)-1-[(1R)-1-(1H-imidazol-2-yl)propyl]-5′,6′-dihydrospiro[pyrrolidine-3,4′-pyrrolo[1,2-b]pyrazol]-2′-yl}pyridin-2-amine
3-(difluoromethoxy)-5-{(3R)-1-[(1S)-1-(1H-imidazol-2-yl)propyl]-5′,6′-dihydrospiro[pyrrolidine-3,4′-pyrrolo[1,2-b]pyrazol]-2′-yl}pyridin-2-amine
2-amino-5-{(3R)-1-[(R)-cyclopropyl(1H-imidazol-2-yl)methyl]-5′,6′-dihydrospiro[pyrrolidine-3,4′-pyrrolo[1,2-b]pyrazol]-2′-yl}nicotinonitrile
2-amino-5-{(3R)-1-[(S)-cyclopropyl(1H-imidazol-2-yl)methyl]-5′,6′-dihydrospiro[pyrrolidine-3,4′-pyrrolo[1,2-b]pyrazol]-2′-yl}nicotinonitrile
2-amino-5-{(3R)-1-[(1R)-1-(1H-imidazol-2-yl)propyl]-5′,6′-dihydrospiro[pyrrolidine-3,4′-pyrrolo[1,2-b]pyrazol]-2′-yl}nicotinonitrile
2-amino-5-{(3R)-1-[(1S)-1-(1H-imidazol-2-yl)propyl]-5′,6′-dihydrospiro[pyrrolidine-3,4′-pyrrolo[1,2-b]pyrazol]-2′-yl}nicotinonitrile
(3R)-2′-(6-amino-5-{[1-(6-methylpyridin-2-yl)ethyl]oxy}pyridin-3-yl)-N-ethyl-5′,6′-dihydrospiro[pyrrolidine-3,4′-pyrrolo[1,2-b]pyrazole]-1-carboxamide (diastereomer 1)
(3R)-2′-(6-amino-5-{[1-(6-methylpyridin-2-yl)ethyl]oxy}pyridin-3-yl)-N-ethyl-5′,6′-dihydrospiro[pyrrolidine-3,4′-pyrrolo[1,2-b]pyrazole]-1-carboxamide (diastereomer 2)
(3R)-2′-(6-amino-5-{[1-(5-methylpyridin-2-yl)ethyl]oxy}pyridin-3-yl)-N-ethyl-5′,6′-dihydrospiro[pyrrolidine-3,4′-pyrrolo[1,2-b]pyrazole]-1-carboxamide (diastereomer 1)
(3R)-2′-(6-amino-5-{[1-(5-methylpyridin-2-yl)ethyl]oxy}pyridin-3-yl)-N-ethyl-5′,6′-dihydrospiro[pyrrolidine-3,4′-pyrrolo[1,2-b]pyrazole]-1-carboxamide (diastereomer 2)
(3R)-2′-(6-amino-5-{[1-(4-methylpyridin-2-yl)ethyl]oxy}pyridin-3-yl)-N-ethyl-5′,6′-dihydrospiro[pyrrolidine-3,4′-pyrrolo[1,2-b]pyrazole]-1-carboxamide (diastereomer 1)
(3R)-2′-(6-amino-5-{[1-(4-methylpyridin-2-yl)ethyl]oxy}pyridin-3-yl)-N-ethyl-5′,6′-dihydrospiro[pyrrolidine-3,4′-pyrrolo[1,2-b]pyrazole]-1-carboxamide (diastereomer 2)
(3R)-2′-(6-amino-5-{[1-(3-methylpyridin-2-yl)ethyl]oxy}pyridin-3-yl)-N-ethyl-5′,6′-dihydrospiro[pyrrolidine-3,4′-pyrrolo[1,2-b]pyrazole]-1-carboxamide (diastereomer 1)
(3R)-2′-(6-amino-5-{[1-(3-methyl-pyridin-2-yl)ethyl]oxy}pyridin-3-yl)-N-ethyl-5′,6′-dihydrospiro[pyrrolidine-3,4′-pyrrolo[1,2-b]pyrazole]-1-carboxamide (diastereomer 2)
(3R)-2′-(6-amino-5-{[1-(1,4-dimethyl-1H-pyrazol-5-yl)ethyl]oxy}pyridin-3-yl)-N-ethyl-5′,6′-dihydrospiro[pyrrolidine-3,4′-pyrrolo[1,2-b]pyrazole]-1-carboxamide (diastereomer 1)
(3R)-2′-(6-amino-5-{[1-(1,4-dimethyl-1H-pyrazol-5-yl)ethyl]oxy}pyridin-3-yl)-N-ethyl-5′,6′-dihydrospiro[pyrrolidine-3,4′-pyrrolo[1,2-b]pyrazole]-1-carboxamide (diastereomer 2)
(3R)-2′-(6-amino-5-{[−1-(1,4-dimethyl-1H-pyrazol-5-yl)ethyl]oxy}pyridin-3-yl)-N-(propan-2-yl)-5′,6′-dihydrospiro[pyrrolidine-3,4′-pyrrolo[1,2-b]pyrazole]-1-carboxamide (diastereomer 1)
(3R)-2′-(6-amino-5-{[1-(1,4-dimethyl-1H-pyrazol-5-yl)ethyl]oxy}pyridin-3-yl)-N-ethyl-5′,6′-dihydrospiro[pyrrolidine-3,4′-pyrrolo[1,2-b]pyrazole]-1-carboxamide (diastereomer 2)
(3R)-2′-{6-amino-5-[(1S)-1-(2,6-difluorophenyl)ethoxy]pyridin-3-yl}-N-ethyl-5′,6′-dihydrospiro[pyrrolidine-3,4′-pyrrolo[1,2-b]pyrazole]-1-carboxamide
(3R)-2′-{6-amino-5-[(1R)-1-(5-fluoropyridin-2-yl)ethoxy]pyridin-3-yl}-N-ethyl-5′,6′-dihydrospiro[pyrrolidine-3,4′-pyrrolo[1,2-b]pyrazole]-1-carboxamide
(3R)-2′-{6-amino-5-[(1R)-1-(3-fluoropyridin-2-yl)ethoxy]pyridin-3-yl}-N-ethyl-5′,6′-dihydrospiro[pyrrolidine-3,4′-pyrrolo[1,2-b]pyrazole]-1-carboxamide
(3R)-2′-{6-amino-5-[(1R)-1-(2,6-difluorophenyl)ethoxy]pyridin-3-yl}-N-ethyl-5′,6′-dihydrospiro[pyrrolidine-3,4′-pyrrolo[1,2-b]pyrazole]-1-carboxamide
(3R)-2′-{6-amino-5-[1-(2-cyanophenyl)ethoxy]pyridin-3-yl}-N-ethyl-5′,6′-dihydro-1H-spiro[pyrrolidine-3,4′-pyrrolo[1,2-b]pyrazole]-1-carboxamide (diastereomer 1 and diastereomer 2)
(3R)-2′-{6-amino-5-[1-(2-cyanophenyl)ethoxy]pyridin-3-yl}-N-ethyl-5′,6′-dihydro-1H-spiro[pyrrolidine-3,4′-pyrrolo[1,2-b]pyrazole]-1-carboxamide (diastereomer 1)
(3R)-2′-{6-amino-5-[1-(2-cyanophenyl)ethoxy]pyridin-3-yl}-N-ethyl-5′,6′-dihydro-1H-spiro[pyrrolidine-3,4′-pyrrolo[1,2-b]pyrazole]-1-carboxamide (diastereomer 2)
(3′R)-2-[6-amino-5-(trifluoromethyl) pyridin-3-yl]-N-[1-(1-ethyl-1H-pyrazol-3-yl)ethyl]-6,7-dihydro-1′H-spiro[pyrazolo[5,1-c][1,4]oxazine-4,3′-pyrrolidine]-1′-carboxamide (diastereomer 1 and diastereomer 2)
(3′R)-2-[6-amino-5-(trifluoromethyl)pyridin-3-yl]-N-[(1-methyl-1H-pyrazol-5-yl)methyl]-6,7-dihydrospiro[pyrazolo[5,1-c][1,4]oxazine-4,3′-pyrrolidine]-1′-carboxamide
(3′R)-2-[6-amino-5-(trifluoromethyl)pyridin-3-yl]-N-[(1-methyl-1H-pyrazol-4-yl)methyl]-6,7-dihydrospiro[pyrazolo[5,1-c][1,4]oxazine-4,3′-pyrrolidine]-1′-carboxamide
(3′R)-2-[6-amino-5-(trifluoromethyl) pyridin-3-yl]-N-[1-(1-methyl-1H-pyrazol-5-yl)ethyl]-6,7-dihydro-1′H-spiro[pyrazolo[5,1-c][1,4]oxazine-4,3′-pyrrolidine]-1′-carboxamide (diastereomer 1 and diastereomer 2)
(3′R)-2-[6-amino-5-(trifluoromethyl)pyridin-3-yl]-N-[e-(1-methyl-1H-pyrazol-5-yl)ethyl]-6,7-dihydro-1′H-spiro[pyrazolo[5,1-c][1,4]oxazine-4,3′-pyrrolidine]-1′-carboxamide (diastereomer 1)
(3′R)-2-[6-amino-5-(trifluoromethyl)pyridin-3-yl]-N-[1-(1-methyl-1H-pyrazol-5-yl)ethyl]-6,7-dihydro-1′H-spiro[pyrazolo[5,1-c][1,4]oxazine-4,3′-pyrrolidine]-1′-carboxamide (diastereomer 2)
(3′R)-2-[6-amino-5-(trifluoromethyl) pyridin-3-yl]-N-[1-(1,3-dimethyl-1H-pyrazol-4-yl)ethyl]-6,7-dihydro
1′H-spiro[pyrazolo[5,1-c][1,4]oxazine-4,3′-pyrrolidine]-1′-carboxamide (diastereomer 1 and diastereomer 2)
(3′R)-2-[6-amino-5-(trifluoromethyl)pyridin-3-yl]-N-[1-(1,3-dimethyl-1H-pyrazol-4-yl)ethyl]-6,7-dihydro 1′H-spiro[pyrazolo[5,1-c][1,4]oxazine-4,3′-pyrrolidine]-1′-carboxamide (diastereomer 1)
(3′R)-2-[6-amino-5-(trifluoromethyl)pyridin-3-yl]-N-[1-(1,3-dimethyl-1H-pyrazol-4-yl)ethyl]-6,7-dihydro 1′H-spiro[pyrazolo[5,1-c][1,4]oxazine-4,3′-pyrrolidine]-1′-carboxamide (diastereomer 2)
(3R)-2′-{6-amino-5-[(1R)-1-(3-cyanophenyl)ethoxy]pyridin-3-yl}-N-ethyl-5′,6′-dihydrospiro[pyrrolidine-3,4′-pyrrolo[1,2-b]pyrazole]-1-carboxamide
(3R)-2′-{6-amino-5-[(1R)-1-(4-cyanophenyl)ethoxy]pyridin-3-yl}-N-ethyl-5′,6′-dihydrospiro[pyrrolidine-3,4′-pyrrolo[1,2-b]pyrazole]-1-carboxamide
(3R)-2′-{6-amino-5-[(1R)-1-(3,5-difluoropyridin-4-yl)ethoxy]pyridin-3-yl}-N-ethyl-5′,6′-dihydrospiro[pyrrolidine-3,4′-pyrrolo[1,2-b]pyrazole]-1-carboxamide
(3R)-2′-[6-amino-5-(1-phenylpropoxy)pyridin-3-yl]-N-ethyl-5′,6′-dihydro-1H-spiro[pyrrolidine-3,4′-pyrrolo[1,2-b]pyrazole]-1-carboxamide (diastereomer 1 and diastereomer 2)
(3R)-2′-[6-amino-5-(1-phenylpropoxy)pyridin-3-yl]-N-ethyl-5′,6′-dihydro-1H-spiro[pyrrolidine-3,4′-pyrrolo[1,2-b]pyrazole]-1-carboxamide (diastereomer 1)
(3R)-2′-[6-amino-5-(1-phenylpropoxy)pyridin-3-yl]-N-ethyl-5′,6′-dihydro-1H-spiro[pyrrolidine-3,4′-pyrrolo[1,2-b]pyrazole]-1-carboxamide (diastereomer 2)
(3R)-2′-{6-amino-5-[(1S)-1-(3-cyanophenyl)ethoxy]pyridin-3-yl}-N-ethyl-5′,6′-dihydrospiro[pyrrolidine-3,4′-pyrrolo[1,2-b]pyrazole]-1-carboxamide
(3R)-2′-{6-amino-5-[(1S)-1-(4-cyanophenyl)ethoxy]pyridin-3-yl}-N-ethyl-5′,6′-dihydrospiro[pyrrolidine-3,4′-pyrrolo[1,2-b]pyrazole]-1-carboxamide
(3R)-2′-{6-amino-5-[(1S)-1-(3,5-difluoropyridin-4-yl)ethoxy]pyridin-3-yl}-N-ethyl-5′,6′-dihydrospiro[pyrrolidine-3,4′-pyrrolo[1,2-b]pyrazole]-1-carboxamide
(3′R)-2-[6-amino-5-(trifluoromethyl)pyridin-3-yl]-N-[1-(1,3,4-trimethyl-1H-pyrazol-5-yl)ethyl]-6,7-dihydro-1′H-spiro[pyrazolo[5,1-c][1,4]oxazine-4,3′-pyrrolidine]-1′-carboxamide (diastereomer 1 and diastereomer 2)
(3′R)-2-[6-amino-5-(trifluoromethyl)pyridin-3-yl]-N-[1-(1,3,4-trimethyl-1H-pyrazol-5-yl)ethyl]-6,7-dihydro-1′H-spiro[pyrazolo[5,1-c][1,4]oxazine-4,3′-pyrrolidine]-1′-carboxamide (diastereomer 1)
(3′R)-2-[6-amino-5-(trifluoromethyl)pyridin-3-yl]-N-[1-(1,3,4-trimethyl-1H-pyrazol-5-yl)ethyl]-6,7-dihydro-1′H-spiro[pyrazolo[5,1-c][1,4]oxazine-4,3′-pyrrolidine]-1′-carboxamide (diastereomer 2)
(3′R)-2-[6-amino-5-(trifluoromethyl) pyridin-3-yl]-N-[1-(1,5-dimethyl-1H-pyrazol-4-yl)ethyl]-6,7-dihydro-1′H-spiro[pyrazolo[5,1-c][1,4]oxazine-4,3′-pyrrolidine]-1′-carboxamide (diastereomer 1 and diastereomer 2)
(3′R)-2-[6-amino-5-(trifluoromethyl) pyridin-3-yl]-N-[1-(1-methyl-1H-pyrazol-4-yl)ethyl]-6,7-dihydro-1′H-spiro[pyrazolo[5,1-c][1,4]oxazine-4,3′-pyrrolidine]-1′-carboxamide (diastereomer 1 and diastereomer 2)
(3R)-2′-{6-amino-5-[(1R)-1-(3-fluoropyridin-2-yl)ethoxy]pyridin-3-yl}-N-(propan-2-yl)-5′,6′-dihydrospiro[pyrrolidine-3,4′-pyrrolo[1,2-b]pyrazole]-1-carboxamide
(3R)-2′-{6-amino-5-[(1R)-1-(3-fluoropyridin-2-yl)ethoxy]pyridin-3-yl}-N-cyclobutyl-5′,6′-dihydrospiro[pyrrolidine-3,4′-pyrrolo[1,2-b]pyrazole]-1-carboxamide
(3R)-2′-{6-amino-5-[(1R)-1-(3,5-difluoropyridin-4-yl)ethoxy]pyridin-3-yl}-N-(propan-2-yl)-5′,6′-dihydrospiro[pyrrolidine-3,4′-pyrrolo[1,2-b]pyrazole]-1-carboxamide
(3R)-2′-{6-amino-5-[(1R)-1-(3,5-difluoropyridin-4-yl)ethoxy]pyridin-3-yl}-N-cyclobutyl-5′,6′-dihydrospiro[pyrrolidine-3,4′-pyrrolo[1,2-b]pyrazole]-1-carboxamide
(3R)-2′-{6-amino-5-[(1R)-1-phenylethoxy]pyridin-3-yl}-N-ethyl-5′,6′-dihydrospiro[pyrrolidine-3,4′-pyrrolo[1,2-b]pyrazole]-1-carboxamide
(3R)-2′-{6-amino-5-[1-(1-methyl-1H-pyrazol-5-yl)ethoxy]pyridin-3-yl}-N-isopropyl-5′,6′-dihydro-1H-spiro[pyrrolidine-3,4′-pyrrolo[1,2-b]pyrazole]-1-carboxamide (diastereomer 1)
(3R)-2′-{6-amino-5-[1-(1-methyl-1H-pyrazol-5-yl)ethoxy]pyridin-3-yl}-N-isopropyl-5′,6′-dihydro-1H-spiro[pyrrolidine-3,4′-pyrrolo[1,2-b]pyrazole]-1-carboxamide (diastereomer 2)
(3R)-2′-{6-amino-5-[1-(1-methyl-1H-pyrazol-5-yl)ethoxy]pyridin-3-yl}-N-cyclobutyl-5′,6′-dihydro-1H-spiro[pyrrolidine-3,4′-pyrrolo[1,2-b]pyrazole]-1-carboxamide (diastereomer 1)
(3R)-2′-{6-amino-5-[1-(1-methyl-1H-pyrazol-5-yl)ethoxy]pyridin-3-yl}-N-cyclobutyl-5′,6′-dihydro-1H-spiro[pyrrolidine-3,4′-pyrrolo[1,2-b]pyrazole]-1-carboxamide (diastereomer 2)
(3R)-2′-{6-amino-5-[(1R)-1-(2-fluorophenyl)ethoxy]pyridin-3-yl}-N-(propan-2-yl)-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[1-(1-methyl-1H-pyrazol-5-yl)ethyl]-5′,6′-dihydro-1H-spiro[azetidine-3,4′-pyrrolo[1,2-b]pyrazole]-1-carboxamide (enantiomer 1 and enantiomer 2)
2′-[6-amino-5-(trifluoromethyl)pyridin-3-yl]-N-[1-(1-methyl-1H-pyrazol-5-yl)ethyl]-5′,6′-dihydro-1H-spiro[azetidine-3,4′-pyrrolo[1,2-b]pyrazole]-1-carboxamide (enantiomer 1)
2′-[6-amino-5-(trifluoromethyl)pyridin-3-yl]-N-[1-(1-methyl-1H-pyrazol-5-yl)ethyl]-5′,6′-dihydro-1H-spiro[azetidine-3,4′-pyrrolo[1,2-b]pyrazole]-1-carboxamide (enantiomer 2)
2′-[6-amino-5-(trifluoromethyl)pyridin-3-yl]-N-[1-(1,3-dimethyl-1H-pyrazol-4-yl)ethyl]-5′,6′-dihydro-1H-spiro[azetidine-3,4′-pyrrolo[1,2-b]pyrazole]-1-carboxamide (enantiomer 1 and enantiomer 2)
2′-[6-amino-5-(trifluoromethyl) pyridin-3-yl]-N-[1-(1,3-dimethyl-1H-pyrazol-4-yl)ethyl]-5′,6′-dihydro-1H-spiro[azetidine-3,4′-pyrrolo[1,2-b]pyrazole]-1-carboxamide (enantiomer 1)
2′-[6-amino-5-(trifluoromethyl) pyridin-3-yl]-N-[1-(1,3-dimethyl-1H-pyrazol-4-yl)ethyl]-5′,6′-dihydro-1H-spiro[azetidine-3,4′-pyrrolo[1,2-b]pyrazole]-1-carboxamide (enantiomer 2)
(3S)-2′-[6-amino-5-(trifluoromethyl)pyridin-3-yl]-N-[1-(1-methyl-1H-pyrazol-5-yl)ethyl]-5′,6′-dihydro-1H-spiro[pyrrolidine-3,4′-pyrrolo[1,2-b]pyrazole]-1-carboxamide (diastereomer 1 and diastereomer 2)
(3S)-2′-[6-amino-5-(trifluoromethyl)pyridin-3-yl]-N-[1-(1,3-dimethyl-1H-pyrazol-4-yl)ethyl]-5′,6′-dihydro-1H-spiro[pyrrolidine-3,4′-pyrrolo[1,2-b]pyrazole]-1-carboxamide (diastereomer 1 and diastereomer 2)
(3R)-2′-{6-amino-5-[(1R)-1-(2,6-difluorophenyl)ethoxy]pyridin-3-yl}-N-(propan-2-yl)-5′,6′-dihydrospiro[pyrrolidine-3,4′-pyrrolo[1,2-b]pyrazole]-1-carboxamide
(3R)-2′-(6-amino-5-{methyl[(1R)-1-phenylethyl]amino}pyridin-3-yl)-N-ethyl-5′,6′-dihydrospiro[pyrrolidine-3,4′-pyrrolo[1,2-b]pyrazole]-1-carboxamide
2′-[6-amino-5-(trifluoromethyl)pyridin-3-yl]-N-[1-(1,3,5-trimethyl-1H-pyrazol-4-yl)ethyl]-5′,6′-dihydro-1H-spiro[azetidine-3,4′-pyrrolo[1,2-b]pyrazole]-1-carboxamide (diastereomer 1 and diastereomer 2)
2′-[6-amino-5-(trifluoromethyl) pyridin-3-yl]-N-[1-(1,3,5-trimethyl-1H-pyrazol-4-yl)ethyl]-5′,6′-dihydro-1H-spiro[azetidine-3,4′-pyrrolo[1,2-b]pyrazole]-1-carboxamide (diastereomer 1)
2′-[6-amino-5-(trifluoromethyl)pyridin-3-yl]-N-[1-(1,3,5-trimethyl-1H-pyrazol-4-yl)ethyl]-5′,6′-dihydro-1H-spiro[azetidine-3,4′-pyrrolo[1,2-b]pyrazole]-1-carboxamide (diastereomer 2)
(3R)-2′-{6-amino-5-[(1R)-1-(4-methylpyridin-2-yl)ethoxy]pyridin-3-yl}-N-(propan-2-yl)-5′,6′-dihydrospiro[pyrrolidine-3,4′-pyrrolo[1,2-b]pyrazole]-1-carboxamide
(3R)-2′-{6-amino-5-[(1R)-1-(4-methylpyridin-2-yl)ethoxy]pyridin-3-yl}-N-cyclobutyl-5′,6′-dihydrospiro[pyrrolidine-3,4′-pyrrolo[1,2-b]pyrazole]-1-carboxamide
(3R)-2′-{6-amino-5-[(1R)-1-(6-methylpyridin-2-yl)ethoxy]pyridin-3-yl}-N-(propan-2-yl)-5′,6′-dihydrospiro[pyrrolidine-3,4′-pyrrolo[1,2-b]pyrazole]-1-carboxamide
(3R)-2′-{6-amino-5-[(1R)-1-(6-methylpyridin-2-yl)ethoxy]pyridin-3-yl}-N-cyclobutyl-5′,6′-dihydrospiro[pyrrolidine-3,4′-pyrrolo[1,2-b]pyrazole]-1-carboxamide
(3S)-2′-[6-amino-5-(trifluoromethyl) pyridin-3-yl]-N-[2-(3-fluoropyridin-2-yl)propan-2-yl]-5′,6′-dihydrospiro[pyrrolidine-3,4′-pyrrolo[1,2-b]pyrazole]-1-carboxamide
(3S)-2′-[6-amino-5-(trifluoromethyl)pyridin-3-yl]-N-[1-(1,3,5-trimethyl-1H-pyrazol-4-yl)ethyl]-5′,6′-dihydro-1H-spiro[pyrrolidine-3,4′-pyrrolo[1,2-b]pyrazole]-1-carboxamide (diastereomer 1 and diastereomer 2)
(3R)-2′-[6-amino-5-(trifluoromethyl) pyridin-3-yl]-N-[1-(1,3,5-trimethyl-1H-pyrazol-4-yl)ethyl]-5′,6′-dihydro-1H-spiro[pyrrolidine-3,4′-pyrrolo[1,2-b]pyrazole]-1-carboxamide (diastereomer 1 and diastereomer 2)
(3R)-2′-[6-amino-5-(trifluoromethyl)pyridin-3-yl]-N-[1-(1,3,5-trimethyl-1H-pyrazol-4-yl)ethyl]-5′,6′-dihydro-1H-spiro[pyrrolidine-3,4′-pyrrolo[1,2-b]pyrazole]-1-carboxamide (diastereomer 1)
(3R)-2′-[6-amino-5-(trifluoromethyl) pyridin-3-yl]-N-[1-(1,3,5-trimethyl-1H-pyrazol-4-yl)ethyl]-5′,6′-dihydro-1H-spiro[pyrrolidine-3,4′-pyrrolo[1,2-b]pyrazole]-1-carboxamide (diastereomer 2)
(2′-{6-amino-5-[(1R)-1-phenylethoxy]pyridin-3-yl}-5′,6′-dihydrospiro[azetidine-3,4′-pyrrolo[1,2-b]pyrazol]-1-yl)(cyclopropyl)methanone
2′-[6-amino-5-(trifluoromethyl)pyridin-3-yl]-N-[1-(1-ethyl-1H-pyrazol-3-yl)ethyl]-5′,6′-dihydro-1H-spiro[azetidine-3,4′-pyrrolo[1,2-b]pyrazole]-1-carboxamide (enantiomer 1)
2′-[6-amino-5-(trifluoromethyl)pyridin-3-yl]-N-[1-(1-ethyl-1H-pyrazol-3-yl)ethyl]-5′,6′-dihydro-1H-spiro[azetidine-3,4′-pyrrolo[1,2-b]pyrazole]-1-carboxamide (enantiomer 2)
(3S)-2′-[6-amino-5-(trifluoromethyl)pyridin-3-yl]-N-[1-(1-ethyl-1H-pyrazol-3-yl)ethyl]-5′,6′-dihydro-1H-spiro[pyrrolidine-3,4′-pyrrolo[1,2-b]pyrazole]-1-carboxamide (diastereomer 1)
(3S)-2′-[6-amino-5-(trifluoromethyl)pyridin-3-yl]-N-[1-(1-ethyl-1H-pyrazol-3-yl)ethyl]-5′,6′-dihydro-1H-spiro[pyrrolidine-3,4′-pyrrolo[1,2-b]pyrazole]-1-carboxamide (diastereomer 2)
5-{(3′R)-1′-[cyclopropyl(1H-1,2,4-triazol-5-yl)methyl]-6,7-dihydrospiro[pyrazolo[5,1-c][1,4]oxazine-4,3′-pyrrolidin]-2-yl}-3-(trifluoromethyl)pyridin-2-amine (diastereomer 1 and diastereomer 2)
5-{(3′R)-1′-[cyclopropyl(1H-1,2,4-triazol-5-yl)methyl]-6,7-dihydrospiro[pyrazolo[5,1-c][1,4]oxazine-4,3′-pyrrolidin]-2-yl}-3-(trifluoromethyl)pyridin-2-amine (diastereomer 1)
5-{(3′R)-1′-[cyclopropyl(1H-1,2,4-triazol-5-yl)methyl]-6,7-dihydrospiro[pyrazolo[5,1-c][1,4]oxazine-4,3′-pyrrolidin]-2-yl}-3-(trifluoromethyl)pyridin-2-amine (diastereomer 2)
5-{(3R)-1-[1-(1H-1,2,4-triazol-5-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[1-(1H-1,2,4-triazol-5-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[1-(1H-1,2,4-triazol-5-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3R)-1-[cyclopropyl(1H-1,2,4-triazol-5-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3′R)-1′-[1-(1H-1,2,4-triazol-5-yl)propyl]-6,7-dihydrospiro[pyrazolo[5,1-c][1,4]oxazine-4,3′-pyrrolidin]-2-yl}-3-(trifluoromethyl)pyridin-2-amine (diastereomer 1 and diastereomer 2)
5-{(3′R)-1′-[1-(1H-1,2,4-triazol-5-yl)propyl]-6,7-dihydrospiro[pyrazolo[5,1-c][1,4]oxazine-4,3′-pyrrolidin]-2-yl}-3-(trifluoromethyl)pyridin-2-amine (diastereomer 1)
5-{(3′R)-1′-[1-(1H-1,2,4-Triazol-5-yl)propyl]-6,7-dihydrospiro[pyrazolo[5,1-c][1,4]oxazine-4,3′-pyrrolidin]-2-yl}-3-(trifluoromethyl)pyridin-2-amine (diastereomer 2)
5-{(3′R)-1′-[2,2-difluoro-1-(1H-imidazol-2-yl)ethyl]-6,7-dihydrospiro[pyrazolo[5,1-c][1,4]oxazine-4,3′-pyrrolidin]-2-yl}-3-(trifluoromethyl)pyridin-2-amine (diastereomer 2)
5-{(3′R)-1′-[2,2-difluoro-1-(1H-imidazol-2-yl)ethyl]-6,7-dihydrospiro[pyrazolo[5,1-c][1,4]oxazine-4,3′-pyrrolidin]-2-yl}-3-(trifluoromethyl)pyridin-2-amine (diastereomer 1)
5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}-3-(difluoromethyl)pyridin-2-amine (diastereomer 1 and diastereomer 2)
5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}-3-(difluoromethyl)pyridin-2-amine (diastereomer 1)
5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}-3-(difluoromethyl)pyridin-2-amine (diastereomer 2)
2-amino-5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}nicotinonitrile (diastereomer 1 and diastereomer 2)
2-amino-5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}nicotinonitrile (diastereomer 1)
2-amino-5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}nicotinonitrile (diastereomer 2)
5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}-3-(difluoromethoxy)pyridin-2-amine (diastereomer 1 and diastereomer 2)
5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}-3-(difluoromethoxy)pyridin-2-amine (diastereomer 1)
5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}-3-(difluoromethoxy)pyridin-2-amine (diastereomer 2)
5-{1-[1-(1H-imidazol-2-yl)propyl]-5′,6′-dihydrospiro[azetidine-3,4′-pyrrolo[1,2-b]pyrazol]-2′-yl}-3-(trifluoromethyl)pyridin-2-amine (enantiomer 1 and enantiomer 2)
5-{1-[1-(1H-imidazol-2-yl)propyl]-5′,6′-dihydrospiro[azetidine-3,4′-pyrrolo[1,2-b]pyrazol]-2′-yl}-3-(trifluoromethyl)pyridin-2-amine (enantiomer 1)
5-{1-[1-(1H-imidazol-2-yl)propyl]-5′,6′-dihydrospiro[azetidine-3,4′-pyrrolo[1,2-b]pyrazol]-2′-yl}-3-(trifluoromethyl)pyridin-2-amine (enantiomer 2)
5-{(3′R)-1′-[cyclobutyl(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine (diastereomer 1 and diastereomer 2)
5-{(3′R)-1′-[cyclobutyl(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine (diastereomer 1)
5-{(3′R)-1′-[cyclobutyl(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine (diastereomer 2)
5-{(3′R)-1′-[1-(1H-imidazol-2-yl)-2-methylpropyl]-6,7-dihydrospiro[pyrazolo[5,1-c][1,4]oxazine-4,3′-pyrrolidin]-2-yl}-3-(trifluoromethyl)pyridin-2-amine (diastereomer 1 and diastereomer 2)
5-{(3′R)-1′-[1-(1H-imidazol-2-yl)-2-methylpropyl]-6,7-dihydrospiro[pyrazolo[5,1-c][1,4]oxazine-4,3′-pyrrolidin]-2-yl}-3-(trifluoromethyl)pyridin-2-amine (diastereomer 1)
5-{(3′R)-1′-[1-(1H-imidazol-2-yl)-2-methylpropyl]-6,7-dihydrospiro[pyrazolo[5,1-c][1,4]oxazine-4,3′-pyrrolidin]-2-yl}-3-(trifluoromethyl)pyridin-2-amine (diastereomer 2)
5-{(3R)-1-[cyclobutyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[cyclobutyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[cyclobutyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3R)-1-[1-(1H-imidazol-2-yl)-2,2-dimethylpropyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{1-[cyclopropyl(1H-imidazol-2-yl)methyl]-5′,6′-dihydrospiro[azetidine-3,4′-pyrrolo[1,2-b]pyrazol]-2′-yl}-3-(trifluoromethyl)pyridin-2-amine (enantiomer 1 and enantiomer 2)
5-{1-[cyclopropyl(1H-imidazol-2-yl)methyl]-5′,6′-dihydrospiro[azetidine-3,4′-pyrrolo[1,2-b]pyrazol]-2′-yl}-3-(trifluoromethyl)pyridin-2-amine (enantiomer 1)
5-{1-[cyclopropyl(1H-imidazol-2-yl)methyl]-5′,6′-dihydrospiro[azetidine-3,4′-pyrrolo[1,2-b]pyrazol]-2′-yl}-3-(trifluoromethyl) pyridin-2-amine (enantiomer 2)
5-{(3R)-1-[2,2-difluoro-1-(1H-imidazol-2-yl)ethyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[2,2-difluoro-1-(1H-imidazol-2-yl)ethyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[2,2-difluoro-1-(1H-imidazol-2-yl)ethyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3S)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3S)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3S)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine (diastereomer 1 and diastereomer 2)
5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine (diastereomer 1)
5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine (diastereomer 2)
2-amino-5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}nicotinonitrile (diastereomer 1 and diastereomer 2)
2-amino-5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}nicotinonitrile (diastereomer 1)
2-amino-5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}nicotinonitrile (diastereomer 2)
5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(difluoromethoxy)pyridin-2-amine (diastereomer 1 and diastereomer 2)
5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(difluoromethoxy)pyridin-2-amine (diastereomer 1)
5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(difluoromethoxy)pyridin-2-amine (diastereomer 2)
2-amino-5-{(3R)-1-[1-(1H-imidazol-2-yl)propyl]-5′,6′-dihydrospiro[pyrrolidine-3,4′-pyrrolo[1,2-b]pyrazol]-2′-yl}nicotinonitrile (diastereomer 1 and diastereomer 2)
2-amino-5-{(3R)-1-[1-(1H-imidazol-2-yl)propyl]-5′,6′-dihydrospiro[pyrrolidine-3,4′-pyrrolo[1,2-b]pyrazol]-2′-yl}nicotinonitrile (diastereomer 1)
2-amino-5-{(3R)-1-[1-(1H-imidazol-2-yl)propyl]-5′,6′-dihydrospiro[pyrrolidine-3,4′-pyrrolo[1,2-b]pyrazol]-2′-yl}nicotinonitrile (diastereomer 2)
5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethoxy)pyridin-2-amine (diastereomer 1 and diastereomer 2)
5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethoxy)pyridin-2-amine (diastereomer 1)
5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethoxy)pyridin-2-amine (diastereomer 2)
3-(difluoromethoxy)-5-{(3R)-1-[1-(1H-imidazol-2-yl)propyl]-5′,6′-dihydrospiro[pyrrolidine-3,4′-pyrrolo[1,2-b]pyrazol]-2′-yl}pyridin-2-amine (diastereomer 1 and diastereomer 2)
3-(difluoromethoxy)-5-{(3R)-1-[1-(1H-imidazol-2-yl)propyl]-5′,6′-dihydrospiro[pyrrolidine-3,4′-pyrrolo[1,2-b]pyrazol]-2′-yl}pyridin-2-amine (diastereomer 1)
3-(difluoromethoxy)-5-{(3R)-1-[1-(1H-imidazol-2-yl)propyl]-5′,6′-dihydrospiro[pyrrolidine-3,4′-pyrrolo[1,2-b]pyrazol]-2′-yl}pyridin-2-amine (diastereomer 2)
5-{(3R)-1-[1-(1H-imidazol-2-yl)propyl]-5′,6′-dihydrospiro[pyrrolidine-3,4′-pyrrolo[1,2-b]pyrazol]-2′-yl}-3-(trifluoromethoxy)pyridin-2-amine (diastereomer 1 and diastereomer 2)
5-{(3R)-1-[1-(1H-imidazol-2-yl)propyl]-5′,6′-dihydrospiro[pyrrolidine-3,4′-pyrrolo[1,2-b]pyrazol]-2′-yl}-3-(trifluoromethoxy)pyridin-2-amine (diastereomer 1)
5-{(3R)-1-[1-(1H-imidazol-2-yl)propyl]-5′,6′-dihydrospiro[pyrrolidine-3,4′-pyrrolo[1,2-b]pyrazol]-2′-yl}-3-(trifluoromethoxy)pyridin-2-amine (diastereomer 2)
5-{(3R)-1-[1-(1H-imidazol-2-yl)-2-methylpropyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[1-(1H-imidazol-2-yl)-2-methylpropyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[1-(1H-imidazol-2-yl)-2-methylpropyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3S)-1-[1-(1H-imidazol-2-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3S)-1-[1-(1H-imidazol-2-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3S)-1-[1-(1H-imidazol-2-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3′R)-1′-[1-(1H-imidazol-2-yl)propyl]-6,7-dihydrospiro[pyrazolo[5,1-c][1,4]oxazine-4,3′-pyrrolidin]-2-yl}-3-(trifluoromethyl)pyridin-2-amine (diastereomer 1 and diastereomer 2)
5-{(3′R)-1′-[1-(1H-imidazol-2-yl)propyl]-6,7-dihydrospiro[pyrazolo[5,1-c][1,4]oxazine-4,3′-pyrrolidin]-2-yl}-3-(trifluoromethyl)pyridin-2-amine (diastereomer 1)
5-{(3′R)-1′-[1-(1H-imidazol-2-yl)propyl]-6,7-dihydrospiro[pyrazolo[5,1-c][1,4]oxazine-4,3′-pyrrolidin]-2-yl}-3-(trifluoromethyl) pyridin-2-amine (diastereomer 2)
{(3R)-2′-[6-amino-5-(trifluoromethyl)pyridin-3-yl]-5′,6′-dihydrospiro[pyrrolidine-3,4′-pyrrolo[1,2-b]pyrazol]-1-yl}(1H-imidazol-2-yl)methanone
{(3R)-2′-[6-amino-5-(trifluoromethyl)pyridin-3-yl]-5′,6′-dihydrospiro[pyrrolidine-3,4′-pyrrolo[1,2-b]pyrazol]-1-yl}(1-methyl-1H-imidazol-2-yl)methanone
3-[(1R)-1-(2,6-dichloro-3-fluorophenyl)ethoxy]-5-{(3R)-1-[(1H-imidazol-2-yl)methyl]-5′,6′-dihydrospiro[pyrrolidine-3,4′-pyrrolo[1,2-b]pyrazol]-2′-yl}pyridin-2-amine
5-{(3R)-1-[1-(1H-imidazol-2-yl)ethyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[1-(1H-imidazol-2-yl)ethyl]-5′,6′-dihydrospiro[pyrrolidine-3,4′-pyrrolo[1,2-b]pyrazol]-2′-yl}-3-(trifluoromethyl)pyridin-2-amine (diastereomer 2)
(3R)-2′-[6-amino-5-(trifluoromethyl)pyridin-3-yl]-N-[1-(3-chloropyridin-4-yl)cyclobutyl]-5′,6′-dihydrospiro[pyrrolidine-3,4′-pyrrolo[1,2-b]pyrazole]-1-carboxamide
(3R)-2′-[6-amino-5-(trifluoromethyl)pyridin-3-yl]-N-[2-(3-chloropyridin-4-yl)propan-2-yl]-5′,6′-dihydrospiro[pyrrolidine-3,4′-pyrrolo[1,2-b]pyrazole]-1-carboxamide
(3R)-2′-[6-amino-5-(trifluoromethyl)pyridin-3-yl]-N-[1-(pyridin-4-yl)cyclobutyl]-5′,6′-dihydrospiro[pyrrolidine-3,4′-pyrrolo[1,2-b]pyrazole]-1-carboxamide
(3R)-2′-[6-amino-5-(trifluoromethyl)pyridin-3-yl]-N-[1-(pyridin-3-yl)cyclobutyl]-5′,6′-dihydrospiro[pyrrolidine-3,4′-pyrrolo[1,2-b]pyrazole]-1-carboxamide
(3R)-2′-[6-amino-5-(trifluoromethyl)pyridin-3-yl]-N-[(1S)-1-(3-chloropyridin-4-yl)ethyl]-5′,6′-dihydrospiro[pyrrolidine-3,4′-pyrrolo[1,2-b]pyrazole]-1-carboxamide
2′-(6-amino-5-cyanopyridin-3-yl)-N-[1-(pyridin-4-yl)cyclobutyl]-5′,6′-dihydrospiro[pyrrolidine-3,4′-pyrrolo[1,2-b]pyrazole]-1-carboxamide (enantiomer 2)
(3R)-2′-[6-amino-5-(trifluoromethyl)pyridin-3-yl]-N-[1-(3-methylpyridin-4-yl)ethyl]-5′,6′-dihydro-1H-spiro[pyrrolidine-3,4′-pyrrolo[1,2-b]pyrazole]-1-carboxamide (diastereomer 1 and diastereomer 2)
(3R)-2′-[6-amino-5-(trifluoromethyl)pyridin-3-yl]-N-[cyclopropyl(phenyl)methyl]-5′,6′-dihydrospiro[pyrrolidine-3,4′-pyrrolo[1,2-b]pyrazole]-1-carboxamide (diastereomer 1)
(3R)-2′-[6-amino-5-(trifluoromethyl)pyridin-3-yl]-N-[cyclopropyl(phenyl)methyl]-5′,6′-dihydrospiro[pyrrolidine-3,4′-pyrrolo[1,2-b]pyrazole]-1-carboxamide (diastereomer 2)
(3R)-2′-[6-amino-5-(trifluoromethyl)pyridin-3-yl]-N-[1-(pyridin-2-yl)cyclobutyl]-5′,6′-dihydrospiro[pyrrolidine-3,4′-pyrrolo[1,2-b]pyrazole]-1-carboxamide
(3R)-2′-[6-amino-5-(trifluoromethyl) pyridin-3-yl]-N-[2-methyl-1-(pyridin-4-yl)propyl]-5′,6′-dihydro-1H-spiro[pyrrolidine-3,4′-pyrrolo[1,2-b]pyrazole]-1-carboxamide (diastereomer 1 and diastereomer 2)
(3R)-2′-[6-amino-5-(trifluoromethyl)pyridin-3-yl]-N-[2-methyl-1-(pyridin-4-yl)propyl]-5′,6′-dihydro-1H spiro[pyrrolidine-3,4′-pyrrolo[1,2-b]pyrazole]-1-carboxamide (diastereomer 1)
(3R)-2′-[6-amino-5-(trifluoromethyl)pyridin-3-yl]-N-[2-methyl-1-(pyridin-4-yl)propyl]-5′,6′-dihydro-1H-spiro[pyrrolidine-3,4′-pyrrolo[1,2-b]pyrazole]-1-carboxamide (diastereomer 2)
(3R)-2′-[6-amino-5-(trifluoromethyl)pyridin-3-yl]-N-[1-(3-fluoropyridin-4-yl)ethyl]-5′,6′-dihydro-1H-spiro[pyrrolidine-3,4′-pyrrolo[1,2-b]pyrazole]-1-carboxamide (diastereomer 1 and diastereomer 2)
(3R)-2′-[6-amino-5-(trifluoromethyl)pyridin-3-yl]-N-[1-(3-fluoropyridin-4-yl)ethyl]-5′,6′-dihydro-1H-spiro[pyrrolidine-3,4′-pyrrolo[1,2-b]pyrazole]-1-carboxamide (diastereomer 1)
(3R)-2′-[6-amino-5-(trifluoromethyl)pyridin-3-yl]-N-[1-(3-fluoropyridin-4-yl)ethyl]-5′,6′-dihydro-1H-spiro[pyrrolidine-3,4′-pyrrolo[1,2-b]pyrazole]-1-carboxamide (diastereomer 2)
(3R)-2′-[6-amino-5-(trifluoromethyl)pyridin-3-yl]-N-[(1R)-1-(3-chloropyridin-4-yl)ethyl]-5′,6′-dihydrospiro[pyrrolidine-3,4′-pyrrolo[1,2-b]pyrazole]-1-carboxamide
(3R)-2′-[6-amino-5-(trifluoromethyl) pyridin-3-yl]-N-[1-(2-methylpyridin-4-yl)ethyl]-5′,6′-dihydro-1H-spiro[pyrrolidine-3,4′-pyrrolo[1,2-b]pyrazole]-1-carboxamide (diastereomer 1 and diastereomer 2)
(3R)-2′-[6-amino-5-(trifluoromethyl) pyridin-3-yl]-N-[1-(3-methoxypyridin-4-yl)ethyl]-5′,6′-dihydro-1H-spiro[pyrrolidine-3,4′-pyrrolo[1,2-b]pyrazole]-1-carboxamide (diastereomer 1 and diastereomer 2)
(3R)-2′-[6-amino-5-(trifluoromethyl)pyridin-3-yl]-N-[1-(pyridin-4-yl)cyclopropyl]-5′,6′-dihydrospiro[pyrrolidine-3,4′-pyrrolo[1,2-b]pyrazole]-1-carboxamide
(3R)-2′-{6-amino-5-[(1R)-1-(2-methoxyphenyl)ethoxy]pyridin-3-yl}-N-ethyl-5′,6′-dihydrospiro[pyrrolidine-3,4′-pyrrolo[1,2-b]pyrazole]-1-carboxamide
3-(1-{(3R)-2′-[6-amino-5-(trifluoromethyl)pyridin-3-yl]-5′,6′-dihydro-1H-spiro[pyrrolidine-3,4′-pyrrolo[1,2-b]pyrazol]-1-yl}propyl)benzonitrile (diastereomer 1 and diastereomer 2)
3-(1-{(3R)-2′-[6-amino-5-(trifluoromethyl)pyridin-3-yl]-5′,6′-dihydro-1H-spiro[pyrrolidine-3,4′-pyrrolo[1,2-b]pyrazol]-1-yl}propyl)benzonitrile (diastereomer 1)
3-(1-{(3R)-2′-[6-amino-5-(trifluoromethyl)pyridin-3-yl]-5′,6′-dihydro-1H-spiro[pyrrolidine-3,4′-pyrrolo[1,2-b]pyrazol]-1-yl}propyl)benzonitrile (diastereomer 2)
(3S)-2′-(6-amino-5-cyanopyridin-3-yl)-N-[2-(3-chloropyridin-4-yl)propan-2-yl]-5′,6′-dihydrospiro[pyrrolidine-3,4′-pyrrolo[1,2-b]pyrazole]-1-carboxamide (enantiomer 1)
(3R)-2′-(6-amino-5-cyanopyridin-3-yl)-N-[2-(3-chloropyridin-4-yl)propan-2-yl]-5′,6′-dihydrospiro[pyrrolidine-3,4′-pyrrolo[1,2-b]pyrazole]-1-carboxamide (enantiomer 2)
(3R)-2′-{6-amino-5-[(1R)-1-(3-chlorophenyl)ethoxy]pyridin-3-yl}-N-(propan-2-yl)-5′,6′-dihydrospiro[pyrrolidine-3,4′-pyrrolo[1,2-b]pyrazole]-1-carboxamide
(3R)-2′-{6-amino-5-[1-(2,6-dichlorophenyl)ethoxy]pyridin-3-yl}-N-ethyl-5′,6′-dihydro-1H-spiro[pyrrolidine-3,4′-pyrrolo[1,2-b]pyrazole]-1-carboxamide (diastereomer 1 and diastereomer 2)
(3R)-2′-{6-amino-5-[1-(2,6-dichlorophenyl)ethoxy]pyridin-3-yl}-N-ethyl-5′,6′-dihydro-1H-spiro[pyrrolidine-3,4′-pyrrolo[1,2-b]pyrazole]-1-carboxamide (diastereomer 1)
(3R)-2′-{6-amino-5-[1-(2,6-dichlorophenyl)ethoxy]pyridin-3-yl}-N-ethyl-5′,6′-dihydro-1H-spiro[pyrrolidine-3,4′-pyrrolo[1,2-b]pyrazole]-1-carboxamide (diastereomer 2)
5-{(3R)-1-[1-(1H-pyrrol-2-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
(3R)-2′-[6-amino-5-(trifluoromethyl)pyridin-3-yl]-N-[1-(2-methoxypyridin-4-yl)ethyl]-5′,6′-dihydro-1H-spiro[pyrrolidine-3,4′-pyrrolo[1,2-b]pyrazole]-1-carboxamide (diastereomer 1 and diastereomer 2)
(3R)-2′-[6-amino-5-(trifluoromethyl)pyridin-3-yl]-N-[1-(1-methyl-1H-pyrazol-5-yl)ethyl]-5′,6′-dihydrospiro[pyrrolidine-3,4′-pyrrolo[1,2-b]pyrazole]-1-carboxamide (diastereomer 1)
(3R)-2′-[6-amino-5-(trifluoromethyl)pyridin-3-yl]-N-[1-(1-methyl-1H-pyrazol-5-yl)ethyl]-5′,6′-dihydrospiro[pyrrolidine-3,4′-pyrrolo[1,2-b]pyrazole]-1-carboxamide (diastereomer 2)
3-(1-{(3R)-2′-[6-amino-5-(trifluoromethyl)pyridin-3-yl]-5′,6′-dihydro-1H-spiro[pyrrolidine-3,4′-pyrrolo[1,2-b]pyrazol]-1-yl}propyl)benzamide (diastereomer 1 and diastereomer 2)
3-(1-{(3R)-2′-[6-amino-5-(trifluoromethyl) pyridin-3-yl]-5′,6′-dihydrospiro[pyrrolidine-3,4′-pyrrolo[1,2-b]pyrazol]-1-yl}propyl)benzamide (diastereomer 1)
3-(1-{(3R)-2′-[6-amino-5-(trifluoromethyl)pyridin-3-yl]-5′,6′-dihydrospiro[pyrrolidine-3,4′-pyrrolo[1,2-b]pyrazol]-1-yl}propyl)benzamide (diastereomer 2)
(3R)-2′-{6-amino-5-[(1R)-1-(1,3-thiazol-2-yl)ethoxy]pyridin-3-yl}-N-cyclobutyl-5′,6′-dihydrospiro[pyrrolidine-3,4′-pyrrolo[1,2-b]pyrazole]-1-carboxamide
(3R)-2′-{6-amino-5-[(1R)-1-(3-chlorophenyl)ethoxy]pyridin-3-yl}-N-ethyl-5′,6′-dihydrospiro[pyrrolidine-3,4′-pyrrolo[1,2-b]pyrazole]-1-carboxamide
(3R)-2′-[6-amino-5-(trifluoromethyl)pyridin-3-yl]-N-[1-(2-chloro-3-fluoropyridin-4-yl)ethyl]-5′,6′-dihydro-1H-spiro[pyrrolidine-3,4′-pyrrolo[1,2-b]pyrazole]-1-carboxamide (diastereomer 1 and diastereomer 2)
(3R)-2′-[6-amino-5-(trifluoromethyl)pyridin-3-yl]-N-{[3-(methylsulfanyl)pyridin-4-yl]methyl}-5′,6′-dihydrospiro[pyrrolidine-3,4′-pyrrolo[1,2-b]pyrazole]-1-carboxamide
(3R)-2′-{6-amino-5-[(1R)-1-(1,3-thiazol-2-yl)ethoxy]pyridin-3-yl}-N-ethyl-5′,6′-dihydrospiro[pyrrolidine-3,4′-pyrrolo[1,2-b]pyrazole]-1-carboxamide
(3R)-2′-[6-amino-5-(trifluoromethyl)pyridin-3-yl]-N-[1-(1,3-dimethyl-1H-pyrazol-4-yl)ethyl]-5′,6′-dihydrospiro[pyrrolidine-3,4′-pyrrolo[1,2-b]pyrazole]-1-carboxamide (diastereomer 1)
(3R)-2′-[6-amino-5-(trifluoromethyl)pyridin-3-yl]-N-[1-(1,3-dimethyl-1H-pyrazol-4-yl)ethyl]-5′,6′-dihydrospiro[pyrrolidine-3,4′-pyrrolo[1,2-b]pyrazole]-1-carboxamide (diastereomer 2)
(3R)-2′-{6-amino-5-[(1R)-1-(3-fluorophenyl)ethoxy]pyridin-3-yl}-N-(propan-2-yl)-5′,6′-dihydrospiro[pyrrolidine-3,4′-pyrrolo[1,2-b]pyrazole]-1-carboxamide
(3R)-2′-[6-amino-5-(trifluoromethyl)pyridin-3-yl]-N-[1-(pyridin-2-yl)cyclopropyl]-5′,6′-dihydrospiro[pyrrolidine-3,4′-pyrrolo[1,2-b]pyrazole]-1-carboxamide
5-{(3R)-1-[1-(pyrimidin-5-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[1-(pyrimidin-5-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[1-(pyrimidin-5-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 2)
(3R)-2′-{6-amino-5-[(1R)-1-(1,3-thiazol-2-yl)ethoxy]pyridin-3-yl}-N-(propan-2-yl)-5′,6′-dihydrospiro[pyrrolidine-3,4′-pyrrolo[1,2-b]pyrazole]-1-carboxamide
(3R)-2′-[6-amino-5-(trifluoromethyl)pyridin-3-yl]-N-[(3-methylpyridin-4-yl)methyl]-5′,6′-dihydrospiro[pyrrolidine-3,4′-pyrrolo[1,2-b]pyrazole]-1-carboxamide
5-{(3R)-1-[1-(6-methoxypyridin-2-yl)ethyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[1-(6-methoxypyridin-2-yl)ethyl]-5′,6′-dihydrospiro[pyrrolidine-3,4′-pyrrolo[1,2-b]pyrazol]-2′-yl}-3-(trifluoromethyl)pyridin-2-amine (diastereomer 2)
(3R)-2′-(6-amino-5-{1-[3-(3-hydroxy-3-methyl but-1-yn-1-yl)phenyl]ethoxy}pyridin-3-yl)-N-ethyl-5′,6′-dihydro-1H-spiro[pyrrolidine-3,4′-pyrrolo[1,2-b]pyrazole]-1-carboxamide (diastereomer 1 and diastereomer 2)
(3R)-2′-{6-amino-5-[(1R)-1-(pyridin-3-yl)ethoxy]pyridin-3-yl}-N-cyclobutyl-5′,6′-dihydrospiro[pyrrolidine-3,4′-pyrrolo[1,2-b]pyrazole]-1-carboxamide
(3R)-2′-{6-amino-5-[1-(1,3-oxazol-2-yl)ethoxy]pyridin-3-yl}-N-ethyl-5′,6′-dihydro-1H-spiro[pyrrolidine-3,4′-pyrrolo[1,2-b]pyrazole]-1-carboxamide (diastereomer 1 and diastereomer 2)
(3R)-2′-{6-amino-5-[(1R)-1-(3-fluorophenyl)ethoxy]pyridin-3-yl}-N-ethyl-5′,6′-dihydrospiro[pyrrolidine-3,4′-pyrrolo[1,2-b]pyrazole]-1-carboxamide
(3R)-2′-[6-amino-5-(6,7-dihydro-5H-cyclopenta[b]pyridin-7-yloxy)pyridin-3-yl]-N-ethyl-5′,6′-dihydro-1H-spiro[pyrrolidine-3,4′-pyrrolo[1,2-b]pyrazole]-1-carboxamide (diastereomer 2)
5-{(3R)-1-[cyclopropyl(5-fluoropyridin-3-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
(3R)-2′-{6-amino-5-[(1R)-1-(pyrimidin-2-yl)ethoxy]pyridin-3-yl}-N-(propan-2-yl)-5′,6′-dihydrospiro[pyrrolidine-3,4′-pyrrolo[1,2-b]pyrazole]-1-carboxamide
(3R)-2′-{6-amino-5-[(1R)-1-(4-fluorophenyl)ethoxy]pyridin-3-yl}-N-ethyl-5′,6′-dihydrospiro[pyrrolidine-3,4′-pyrrolo[1,2-b]pyrazole]-1-carboxamide
(3R)-2′-{6-amino-5-[(1R)-1-(4-fluorophenyl)ethoxy]pyridin-3-yl}-N-(propan-2-yl)-5′,6′-dihydrospiro[pyrrolidine-3,4′-pyrrolo[1,2-b]pyrazole]-1-carboxamide
5-{(3R)-1-[1-(1,3-thiazol-2-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[1-(1,3-thiazol-2-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[1-(1,3-thiazol-2-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 2)
(3R)-2′-{6-amino-5-[(1R)-1-(4-chlorophenyl)ethoxy]pyridin-3-yl}-N-ethyl-5′,6′-dihydrospiro[pyrrolidine-3,4′-pyrrolo[1,2-b]pyrazole]-1-carboxamide
5-{(3R)-1-[1-(dimethylamino)propan-2-yl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
(3R)-2′-{6-amino-5-[(1R)-1-(1,2-thiazol-5-yl)ethoxy]pyridin-3-yl}-N-ethyl-5′,6′-dihydrospiro[pyrrolidine-3,4′-pyrrolo[1,2-b]pyrazole]-1-carboxamide
6-({(3R)-2′-[6-amino-5-(trifluoromethyl)pyridin-3-yl]-5′,6′-dihydrospiro[pyrrolidine-3,4′-pyrrolo[1,2-b]pyrazol]-1-yl}methyl)pyridin-2(1H)-one
(3R)-2′-{6-amino-5-[(1R)-1-(pyridin-3-yl)ethoxy]pyridin-3-yl}-N-(propan-2-yl)-5′,6′-dihydrospiro[pyrrolidine-3,4′-pyrrolo[1,2-b]pyrazole]-1-carboxamide
2′-[6-amino-5-(6,7-dihydro-5H-cyclopenta[b]pyridin-7-yloxy)pyridin-3-yl]-N-ethyl-5′,6′-dihydro-1H-spiro[pyrrolidine-3,4′-pyrrolo[1,2-b]pyrazole]-1-carboxamide (4 isomers)
(3R)-2′-{6-amino-5-[1-(1,3-oxazol-4-yl)ethoxy]pyridin-3-yl}-N-isopropyl-5′,6′-dihydro-1H-spiro[pyrrolidine-3,4′-pyrrolo[1,2-b]pyrazole]-1-carboxamide (diastereomer 1 and diastereomer 2)
5-{(3R)-1-[(1,3-thiazol-2-yl)methyl]-5′,6′-dihydrospiro[pyrrolidine-3,4′-pyrrolo[1,2-b]pyrazol]-2′-yl}-3-(trifluoromethyl)pyridin-2-amine
6-({(3′R)-2-[6-amino-5-(trifluoromethyl)pyridin-3-yl]-6,7-dihydrospiro[pyrazolo[5,1-c][1,4]oxazine-4,3′-pyrrolidin]-1′-yl}methyl)pyridin-2(1H)-one
(3S)-2′-[6-amino-5-(trifluoromethyl)pyridin-3-yl]-N-[cyclopropyl(phenyl)methyl]-5′,6′-dihydrospiro[pyrrolidine-3,4′-pyrrolo[1,2-b]pyrazole]-1-carboxamide (diastereomer 1)
(3S)-2′-[6-amino-5-(trifluoromethyl)pyridin-3-yl]-N-[cyclopropyl(phenyl)methyl]-5′,6′-dihydrospiro[pyrrolidine-3,4′-pyrrolo[1,2-b]pyrazole]-1-carboxamide (diastereomer 2)
(3R)-2′-[6-amino-5-(6,7-dihydro-5H-cyclopenta[b]pyridin-7-yloxy)pyridin-3-yl]-N-ethyl-5′,6′-dihydro-1H-spiro[pyrrolidine-3,4′-pyrrolo[1,2-b]pyrazole]-1-carboxamide (diastereomer 1)
2′-{6-amino-5-[(1R)-1-(pyridin-2-yl)ethoxy]pyridin-3-yl}-N-ethyl-5′,6′-dihydrospiro[azetidine-3,4′-pyrrolo[1,2-b]pyrazole]-1-carboxamide
1-{(3R)-2′-[6-amino-5-(trifluoromethyl)pyridin-3-yl]-5′,6′-dihydrospiro[pyrrolidine-3,4′-pyrrolo[1,2-b]pyrazol]-1-yl}-2-(methylamino)ethan-1-one
(2R)-1-{(3R)-2′-[6-amino-5-(trifluoromethyl) pyridin-3-yl]-5′,6′-dihydrospiro[pyrrolidine-3,4′-pyrrolo[1,2-b]pyrazol]-1-yl}-2-(methylamino)propan-1-one
(5R)-5-({(3R)-2′-[6-amino-5-(trifluoromethyl)pyridin-3-yl]-5′,6′-dihydrospiro[pyrrolidine-3,4′-pyrrolo[1,2-b]pyrazol]-1-yl}methyl)pyrrolidin-2-one
5-{(3R)-1-[(1,3,4-thiadiazol-2-yl)methyl]-5′,6′-dihydrospiro[pyrrolidine-3,4′-pyrrolo[1,2-b]pyrazol]-2′-yl}-3-(trifluoromethyl)pyridin-2-amine
(5S)-5-({(3R)-2′-[6-amino-5-(trifluoromethyl)pyridin-3-yl]-5′,6′-dihydrospiro[pyrrolidine-3,4′-pyrrolo[1,2-b]pyrazol]-1-yl}methyl)pyrrolidin-2-one
5-{(3′R)-1′-[1-(6-methoxypyridin-2-yl)ethyl]-6,7-dihydrospiro[pyrazolo[5,1-c][1,4]oxazine-4,3′-pyrrolidin]-2-yl}-3-(trifluoromethyl)pyridin-2-amine (diastereomer 1 and diastereomer 2)
5-{(3R)-1-[cyclopropyl(6-methoxypyridin-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3′R)-1′-[cyclopropyl(6-methoxypyridin-2-yl)methyl]-6,7-dihydrospiro[pyrazolo[5,1-c][1,4]oxazine-4,3′-pyrrolidin]-2-yl}-3-(trifluoromethyl)pyridin-2-amine (diastereomer 1 and diastereomer 2)
5-{(3R)-1-[1-(3-fluoropyridin-2-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
3-(difluoromethoxy)-5-{(3′R)-1′-[1-(1H-imidazol-2-yl)propyl]-6,7-dihydrospiro[pyrazolo[5,1-c][1,4]oxazine-4,3′-pyrrolidin]-2-yl}pyridin-2-amine (diastereomer 1 and diastereomer 2)
3-(difluoromethoxy)-5-{(3′R)-1′-[1-(1H-imidazol-2-yl)propyl]-6,7-dihydrospiro[pyrazolo[5,1-c][1,4]oxazine-4,3′-pyrrolidin]-2-yl}pyridin-2-amine (diastereomer 1)
3-(difluoromethoxy)-5-{(3′R)-1′-[1-(1H-imidazol-2-yl)propyl]-6,7-dihydrospiro[pyrazolo[5,1-c][1,4]oxazine-4,3′-pyrrolidin]-2-yl}pyridin-2-amine (diastereomer 2)
2-amino-5-{(3′R)-1′-[1-(1H-imidazol-2-yl)propyl]-6,7-dihydrospiro[pyrazolo[5,1-c][1,4]oxazine-4,3′-pyrrolidin]-2-yl}pyridine-3-carbonitrile (diastereomer 1 and diastereomer 2)
2-amino-5-{(3′R)-1′-[1-(1H-imidazol-2-yl)propyl]-6,7-dihydrospiro[pyrazolo[5,1-c][1,4]oxazine-4,3′-pyrrolidin]-2-yl}pyridine-3-carbonitrile (diastereomer 1)
2-amino-5-{(3′R)-1′-[1-(1H-imidazol-2-yl)propyl]-6,7-dihydrospiro[pyrazolo[5,1-c][1,4]oxazine-4,3′-pyrrolidin]-2-yl}pyridine-3-carbonitrile (diastereomer 2)
3-(difluoromethyl)-5-{(3′R)-1′-[1-(1H-imidazol-2-yl)propyl]-6,7-dihydrospiro[pyrazolo[5,1-c][1,4]oxazine-4,3′-pyrrolidin]-2-yl}pyridin-2-amine (diastereomer 1 and diastereomer 2)
3-(difluoromethyl)-5-{(3′R)-1′-[1-(1H-imidazol-2-yl)propyl]-6,7-dihydrospiro[pyrazolo[5,1-c][1,4]oxazine-4,3′-pyrrolidin]-2-yl}pyridin-2-amine (diastereomer 1)
3-(difluoromethyl)-5-{(3′R)-1′-[1-(1H-imidazol-2-yl)propyl]-6,7-dihydrospiro[pyrazolo[5,1-c][1,4]oxazine-4,3′-pyrrolidin]-2-yl}pyridin-2-amine (diastereomer 2)
(3R)-2′-(6-amino-5-{[1-(1-propyl-1H-pyrazol-5-yl)ethyl]oxy}pyridin-3-yl)-N-ethyl-5′,6′-dihydrospiro[pyrrolidine-3,4′-pyrrolo[1,2-b]pyrazole]-1-carboxamide (diastereomer 1 and diastereomer 2)
(3R)-2′-(6-amino-5-{[1-(1,3-thiazol-4-yl)ethyl]oxy}pyridin-3-yl)-N-ethyl-5′,6′-dihydrospiro[pyrrolidine-3,4′-pyrrolo[1,2-b]pyrazole]-1-carboxamide (diastereomer 1 and diastereomer 2)
(3R)-2′-(6-amino-5-{[1-(pyrimidin-4-yl)ethyl]oxy}pyridin-3-yl)-N-(propan-2-yl)-5′,6′-dihydrospiro[pyrrolidine-3,4′-pyrrolo[1,2-b]pyrazole]-1-carboxamide (diastereomer 1 and diastereomer 2)
(3R)-2′-(6-amino-5-{[1-(2,4-dimethylpyrimidin-5-yl)ethyl]oxy}pyridin-3-yl)-N-(propan-2-yl)-5′,6′-dihydrospiro[pyrrolidine-3,4′-pyrrolo[1,2-b]pyrazole]-1-carboxamide (diastereomer 1)
(3R)-2′-(6-amino-5-{[1-(2,4-dimethylpyrimidin-5-yl)ethyl]oxy}pyridin-3-yl)-N-(propan-2-yl)-5′,6′-dihydrospiro[pyrrolidine-3,4′-pyrrolo[1,2-b]pyrazole]-1-carboxamide (diastereomer 2)
(3R)-2′-{6-amino-5-[(1R)-1-(5-methyl-1,3,4-oxadiazol-2-yl)ethoxy]pyridin-3-yl}-N-(propan-2-yl)-5′,6′-dihydrospiro[pyrrolidine-3,4′-pyrrolo[1,2-b]pyrazole]-1-carboxamide
(3R)-2′-{6-amino-5-[(1R)-1-(1-methyl-1H-1,2,3-triazol-5-yl)ethoxy]pyridin-3-yl}-N-(propan-2-yl)-5′,6′-dihydrospiro[pyrrolidine-3,4′-pyrrolo[1,2-b]pyrazole]-1-carboxamide
(3R)-2′-(6-amino-5-{[1-(1-methyl-1H-1,2,3-triazol-4-yl)ethyl]oxy}pyridin-3-yl)-N-(propan-2-yl)-5′,6′-dihydrospiro[pyrrolidine-3,4′-pyrrolo[1,2-b]pyrazole]-1-carboxamide (diastereomer 1 and diastereomer 2)
(5S)-5-{[(3R)-2′-{6-amino-5-[(1R)-1-phenylethoxy]pyridin-3-yl}-5′,6′-dihydrospiro[pyrrolidine-3,4′-pyrrolo[1,2-b]pyrazol]-1-yl]methyl}pyrrolidin-2-one
(5R)-5-{[(3R)-2′-{6-amino-5-[(1R)-1-phenylethoxy]pyridin-3-yl}-5′,6′-dihydrospiro[pyrrolidine-3,4′-pyrrolo[1,2-b]pyrazol]-1-yl]methyl}pyrrolidin-2-one
(3R)-2′-(6-amino-5-{1-[4-(methylsulfonyl)phenyl]ethoxy}pyridin-3-yl)-N-isopropyl-5′,6′-dihydro-1H-spiro[pyrrolidine-3,4′-pyrrolo[1,2-b]pyrazole]-1-carboxamide (diastereomer 1 and diastereomer 2)
(3R)-2′-{6-amino-5-[(1R)-1-(2-methyl-1,3-thiazol-4-yl)ethoxy]pyridin-3-yl}-N-(propan-2-yl)-5′,6′-dihydrospiro[pyrrolidine-3,4′-pyrrolo[1,2-b]pyrazole]-1-carboxamide
(3R)-2′-[6-amino-5-({1-[3-(methanesulfonyl)phenyl]ethyl}oxy)pyridin-3-yl]-N-(propan-2-yl)-5′,6′-dihydrospiro[pyrrolidine-3,4′-pyrrolo[1,2-b]pyrazole]-1-carboxamide (diastereomer 1 and diastereomer 2)
(3R)-2′-(6-amino-5-{[1-(4-methyl-1,3-thiazol-2-yl)ethyl]oxy}pyridin-3-yl)-N-ethyl-5′,6′-dihydrospiro[pyrrolidine-3,4′-pyrrolo[1,2-b]pyrazole]-1-carboxamide (diastereomer 1 and diastereomer 2)
(3R)-2′-(6-amino-5-{[1-(4,5-dimethyl-1,3-thiazol-2-yl)ethyl]oxy}pyridin-3-yl)-N-ethyl-5′,6′-dihydrospiro[pyrrolidine-3,4′-pyrrolo[1,2-b]pyrazole]-1-carboxamide (diastereomer 1 and diastereomer 2)
(3R)-2′-(6-amino-5-{[1-(5-methyl-1,3-thiazol-2-yl)ethyl]oxy}pyridin-3-yl)-N-ethyl-5′,6′-dihydrospiro[pyrrolidine-3,4′-pyrrolo[1,2-b]pyrazole]-1-carboxamide (diastereomer 1 and diastereomer 2)
(3R)-2′-{6-amino-5-[(1R)-1-(pyridin-4-yl)ethoxy]pyridin-3-yl}-N-(propan-2-yl)-5′,6′-dihydrospiro[pyrrolidine-3,4′-pyrrolo[1,2-b]pyrazole]-1-carboxamide
(3R)-2′-{6-amino-5-[(1R)-1-(pyridin-4-yl)ethoxy]pyridin-3-yl}-N-cyclobutyl-5′,6′-dihydrospiro[pyrrolidine-3,4′-pyrrolo[1,2-b]pyrazole]-1-carboxamide
6-(1-{(3′R)-2-[6-amino-5-(trifluoromethyl)pyridin-3-yl]-6,7-dihydrospiro[pyrazolo[5,1-c][1,4]oxazine-4,3′-pyrrolidin]-1′-yl}ethyl)pyridin-2-ol (diastereomer 1 and diastereomer 2)
6-(1-{(3′R)-2-[6-amino-5-(trifluoromethyl)pyridin-3-yl]-6,7-dihydrospiro[pyrazolo[5,1-c][1,4]oxazine-4,3′-pyrrolidin]-1′-yl}ethyl)pyridin-2-ol (diastereomer 1)
6-(1-{(3′R)-2-[6-amino-5-(trifluoromethyl)pyridin-3-yl]-6,7-dihydrospiro[pyrazolo[5,1-c][1,4]oxazine-4,3′-pyrrolidin]-1′-yl}ethyl)pyridin-2-ol (diastereomer 2)
6-({(3R)-2′-[6-amino-5-(trifluoromethyl)pyridin-3-yl]-5′,6′-dihydrospiro[pyrrolidine-3,4′-pyrrolo[1,2-b]pyrazol]-1-yl}(cyclopropyl)methyl)pyridin-2-oI (diastereomer 1 and diastereomer 2)
6-({(3′R)-2-[6-amino-5-(trifluoromethyl)pyridin-3-yl]-6,7-dihydrospiro[pyrazolo[5,1-c][1,4]oxazine-4,3′-pyrrolidin]-1′-yl}(cyclopropyl)methyl)pyridin-2-ol (diastereomer 1 and diastereomer 2)
5-{(3R)-1-[cyclopropyl(1H-1,2,4-triazol-5-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[cyclopropyl(1H-1,2,4-triazol-5-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3R)-1-[(3,3-difluorocyclobutyl)(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[(3,3-difluorocyclobutyl)(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[(3,3-difluorocyclobutyl)(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{1′-[(3,3-difluorocyclobutyl)(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine (isomer 1)
5-{1′-[(3,3-difluorocyclobutyl)(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine (isomer 2)
5-{1′-[(3,3-difluorocyclobutyl)(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine (isomer 3)
5-{1′-[(3,3-difluorocyclobutyl)(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine (isomer 4)
5-{(3R)-1-[1-(1H-pyrazol-3-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[(1H-imidazol-2-yl)(1-methylcyclopropyl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[(1H-imidazol-2-yl)(1-methylcyclopropyl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[(1H-imidazol-2-yl)(1-methylcyclopropyl)methyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3R)-1-[cyclobutyl(4H-1,2,4-triazol-3-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[cyclobutyl(4H-1,2,4-triazol-3-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[cyclobutyl(4H-1,2,4-triazol-3-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3′R)-1′-[cyclohexyl(1H-imidazol-2-yl)methyl]-6,7-dihydrospiro[pyrazolo[5,1-c][1,4]oxazine-4,3′-pyrrolidin]-2-yl}-3-(trifluoromethyl)pyridin-2-amine (diastereomer 1 and diastereomer 2)
5-{(3R)-1-[cyclohexyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[1-(4-chloro-1H-imidazol-2-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[(3,3-difluorocyclobutyl)(4H-1,2,4-triazol-3-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3′R)-1′-[cyclopropyl(1H-imidazol-2-yl)methyl]-6,7-dihydrospiro[pyrazolo[5,1-c][1,4]oxazine-4,3′-pyrrolidin]-2-yl}-3-(trifluoromethyl)pyrazin-2-amine (diastereomer 1 and diastereomer 2)
5-{(3R)-1-[(1H-imidazol-2-yl)(1-methylcyclobutyl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[1-(1H-pyrazol-3-yl)propyl]-5′,6′-dihydrospiro[pyrrolidine-3,4′-pyrrolo[1,2-b]pyrazol]-2′-yl}-3,4′-(trifluoromethyl)pyridin-2-amine (diastereomer 1)
5-{(3R)-1-[1-(1H-pyrazol-3-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3R)-1-[cyclopropyl(1H-1,2,3-triazol-5-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[cyclopropyl(1H-1,2,4-triazol-5-yl)methyl]-5′,6′-dihydrospiro[pyrrolidine-3,4′-pyrrolo[1,2-b]pyrazol]-2′-yl}-3-[(1R)-1-(2,6-difluorophenyl)ethoxy]pyridin-2-amine (diastereomer 1 and diastereomer 2)
5-{(3R)-1-[(1-methylcyclobutyl)(4H-1,2,4-triazol-3-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[1-(4-chloro-1H-imidazol-2-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[1-(4-chloro-1H-imidazol-2-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3R)-1-[cyclopropyl(1H-1,2,4-triazol-5-yl)methyl]-5′,6′-dihydrospiro[pyrrolidine-3,4′-pyrrolo[1,2-b]pyrazol]-2′-yl}-3-[(1R)-1-(3-fluoropyridin-2-yl)ethoxy]pyridin-2-amine (diastereomer 1 and diastereomer 2)
5-{(3R)-1-[(3,3-difluorocyclobutyl)(4H-1,2,4-triazol-3-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[(3,3-difluorocyclobutyl)(4H-1,2,4-triazol-3-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3R)-1-[cyclopentyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3′R)-1′-[cyclopropyl(1H-1,2,3-triazol-5-yl)methyl]-6,7-dihydrospiro[pyrazolo[5,1-c][1,4]oxazine-4,3′-pyrrolidin]-2-yl}-3-(trifluoromethyl)pyridin-2-amine (diastereomer 1 and diastereomer 2)
5-{(3R)-1-[cyclopropyl(1-methyl-1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3R)-1-[1-(4H-1,2,4-triazol-3-yl)(1-²H)propyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[1-(4H-1,2,4-triazol-3-yl)(1-²H)propyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3R)-1-[1-(4H-1,2,4-triazol-3-yl)(1-²H)propyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3′R)-1′-[1-(4H-1,2,4-triazol-3-yl)(1-²H)propyl]-6,7-dihydrospiro[pyrazolo[5,1-c][1,4]oxazine-4,3′-pyrrolidin]-2-yl}-3-(trifluoromethyl)pyridin-2-amine (diastereomer 1 and diastereomer 2)
5-{(3′R)-1′-[cyclopropyl(1H-imidazol-2-yl)(²H)methyl]-6,7-dihydrospiro[pyrazolo[5,1-c][1,4]oxazine-4,3′-pyrrolidin]-2-yl}-3-(trifluoromethyl)pyridin-2-amine (diastereomer 1 and diastereomer 2)
5-{(3′R)-1′-[cyclopropyl(1H-imidazol-2-yl)(²H)methyl]-6,7-dihydrospiro[pyrazolo[5,1-c][1,4]oxazine-4,3′-pyrrolidin]-2-yl}-3-(trifluoromethyl)pyridin-2-amine (diastereomer 1)
5-{(3′R)-1′-[cyclopropyl(1H-imidazol-2-yl)(²H)methyl]-6,7-dihydrospiro[pyrazolo[5,1-c][1,4]oxazine-4,3′-pyrrolidin]-2-yl}-3-(trifluoromethyl)pyridin-2-amine (diastereomer 2)
5-{(3′R)-1′-[1-(1H-imidazol-2-yl)(1-²H)propyl]-6,7-dihydrospiro[pyrazolo[5,1-c][1,4]oxazine-4,3′-pyrrolidin]-2-yl}-3-(trifluoromethyl)pyridin-2-amine (diastereomer 1 and diastereomer 2)
5-{(3′R)-1′-[1-(1H-imidazol-2-yl)(1-²H)propyl]-6,7-dihydrospiro[pyrazolo[5,1-c][1,4]oxazine-4,3′-pyrrolidin]-2-yl}-3-(trifluoromethyl)pyridin-2-amine (diastereomer 1)
5-{(3′R)-1′-[1-(1H-imidazol-2-yl)(1-²H)propyl]-6,7-dihydrospiro[pyrazolo[5,1-c][1,4]oxazine-4,3′-pyrrolidin]-2-yl}-3-(trifluoromethyl)pyridin-2-amine (diastereomer 2)
5-{(3R)-1-[1-(1H-imidazol-2-yl)(1-2H)propyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[1-(1H-imidazol-2-yl)(1-²H)propyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[1-(1H-imidazol-2-yl)(1-2H)propyl]-5′,6′-dihydrospiro[pyrrolidine-3,4′-pyrrolo[1,2-b]pyrazol]-2′-yl}-3-(trifluoromethyl) pyridin-2-amine (diastereomer 2)
5-{(3R)-1-[cyclopropyl(1H-imidazol-2-yl)(2H)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[cyclopropyl(1H-imidazol-2-yl)(²H)methyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[cyclopropyl(1H-imidazol-2-yl)(²H)methyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3R)-1-[cyclopropyl(2-methoxypyridin-3-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
3-[{(3R)-2′-[6-amino-5-(trifluoromethyl)pyridin-3-yl]-5′,6′-dihydro-1H-spiro[pyrrolidine-3,4′-pyrrolo[1,2-b]pyrazol]-1-yl}(cyclopropyl)methyl]pyridin-2(1H)-one (diastereomer 1 and diastereomer 2)
5-{(3R)-1-[(1-fluorocyclopropyl)(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[(1-fluorocyclopropyl)(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[(1-fluorocyclopropyl)(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3R)-1-[cyclopropyl(4-methoxypyrimidin-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[cyclobutyl(1H-1,2,4-triazol-5-yl)methyl]-5′,6′-dihydrospiro[pyrrolidine-3,4′-pyrrolo[1,2-b]pyrazol]-2′-yl}-3-(trifluoromethoxy)pyridin-2-amine (diastereomer 1 and diastereomer 2)
5-{(3R)-1-[cyclopropyl(1H-1,2,4-triazol-5-yl)methyl]-5′,6′-dihydrospiro[pyrrolidine-3,4′-pyrrolo[1,2-b]pyrazol]-2′-yl}-3-(trifluoromethoxy)pyridin-2-amine (diastereomer 1 and diastereomer 2)
5-{(3′R)-1′-[cyclopropyl(1H-1,2,3-triazol-5-yl)methyl]-6,7-dihydrospiro[pyrazolo[5,1-c][1,4]oxazine-4,3′-pyrrolidin]-2-yl}-3-(trifluoromethyl)pyridin-2-amine (diastereomer 1)
5-{(3′R)-1′-[cyclopropyl(1H-1,2,3-triazol-5-yl)methyl]-6,7-dihydrospiro[pyrazolo[5,1-c][1,4]oxazine-4,3′-pyrrolidin]-2-yl}-3-(trifluoromethyl)pyridin-2-amine (diastereomer 2)
5-{(3R)-1-[(1-fluorocyclobutyl)(4H-1,2,4-triazol-3-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[cyclopropyl(pyrimidin-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[cyclopropyl(1H-1,2,4-triazol-5-yl)methyl]-5′,6′-dihydrospiro[pyrrolidine-3,4′-pyrrolo[1,2-b]pyrazol]-2′-yl}-3-[(1R)-1-(2,6-difluorophenyl)ethoxy]pyridin-2-amine (diastereomer 1)
5-{(3R)-1-[cyclopropyl(1H-1,2,4-triazol-5-yl)methyl]-5′,6′-dihydrospiro[pyrrolidine-3,4′-pyrrolo[1,2-b]pyrazol]-2′-yl}-3-[(1R)-1-(2,6-difluorophenyl)ethoxy]pyridin-2-amine (diastereomer 2)
5-{(3R)-1-[cyclopropyl(1H-1,2,4-triazol-5-yl)methyl]-5′,6′-dihydrospiro[pyrrolidine-3,4′-pyrrolo[1,2-b]pyrazol]-2′-yl}-3-[(1R)-1-(3-fluoropyridin-2-yl)ethoxy]pyridin-2-amine (diastereomer 1)
5-{(3R)-1-[cyclopropyl(1H-1,2,4-triazol-5-yl)methyl]-5′,6′-dihydrospiro[pyrrolidine-3,4′-pyrrolo[1,2-b]pyrazol]-2′-yl}-3-[(1R)-1-(3-fluoropyridin-2-yl)ethoxy]pyridin-2-amine (diastereomer 2)
5-{(3′R)-1′-[cyclobutyl(4H-1,2,4-triazol-3-yl)methyl]-6,7-dihydrospiro[pyrazolo[5,1-c][1,4]oxazine-4,3′-pyrrolidin]-2-yl}-3-(trifluoromethyl)pyridin-2-amine (diastereomer 1)
5-{(3R)-1-[cyclopentyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[cyclopentyl(1H-imidazol-2-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3R)-1-[(4,4-difluorocyclohexyl)(1H-1,2,4-triazol-5-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[(4,4-difluorocyclohexyl)(1H-1,2,4-triazol-5-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[(4,4-difluorocyclohexyl)(1H-1,2,4-triazol-5-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3R)-1-[2,2-difluoro-1-(1H-imidazol-2-yl)propyl]-5′,6′-dihydrospiro[pyrrolidine-3,4′-pyrrolo[1,2-b]pyrazol]-2′-yl}-3-(trifluoromethyl)pyridin-2-amine (diastereomer 1 and diastereomer 2)
5-{(3R)-1-[cyclopropyl(1H-1,2,3-triazol-5-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 1)
5-{(3R)-1-[cyclopropyl(1H-1,2,3-triazol-5-yl)methyl]-5′,6′-dihydrospiro[pyrrolidine-3,4′-pyrrolo[1,2-b]pyrazol]-2′-yl}-3-(trifluoromethyl)pyridin-2-amine (diastereomer 2)
5-{(3′R)-1′-[cyclobutyl(4H-1,2,4-triazol-3-yl)methyl]-6,7-dihydrospiro[pyrazolo[5,1-c][1,4]oxazine-4,3′-pyrrolidin]-2-yl}-3-(trifluoromethyl)pyridin-2-amine (diastereomer 1 and diastereomer 2)
5-{(3R)-1-[cyclopropyl(1H-1,2,4-triazol-5-yl)methyl]-5′,6′-dihydrospiro[pyrrolidine-3,4′-pyrrolo[1,2-b]pyrazol]-2′-yl}-3-(trifluoromethoxy)pyridin-2-amine (diastereomer 1)
5-{(3R)-1-[cyclopropyl(1H-1,2,4-triazol-5-yl)methyl]-5′,6′-dihydrospiro[pyrrolidine-3,4′-pyrrolo[1,2-b]pyrazol]-2′-yl}-3-(trifluoromethoxy)pyridin-2-amine (diastereomer 2)
(3R)-2′-{5-amino-6-[(1R)-1-phenylethoxy]pyrazin-2-yl}-N-ethyl-5′,6′-dihydrospiro[pyrrolidine-3,4′-pyrrolo[1,2-b]pyrazole]-1-carboxamide
5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyrazin-2-amine (diastereomer 1 and diastereomer 2)
5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyrazin-2-amine (diastereomer 1)
5-{(3R)-1-[cyclopropyl(1H-imidazol-2-yl)methyl]-5′,6′-dihydrospiro[pyrrolidine-3,4′-pyrrolo[1,2-b]pyrazol]-2′-yl}-3-(trifluoromethyl)pyrazin-2-amine (diastereomer 2)
or a stereoisomer, a tautomer, an N-oxide, a hydrate, a solvate, or a salt thereof, or a mixture of same.

10. A compound of general formula (I) according to any one of claims 1, 2, 3, 4, 5, 6, 7, 8 or 9 for the use as a medicament.

11. A compound of general formula (I) according to any one of claims 1, 2, 3, 4, 5, 6, 7, 8 or 9 for use in the treatment or prophylaxis of a disease.

12. A pharmaceutical composition comprising a compound of general formula (I) according to any one of claims 1, 2, 3, 4, 5, 6, 7, 8 or 9 and one or more pharmaceutically acceptable excipients.

13. A pharmaceutical combination comprising:
one or more first active ingredients, in particular compounds of general formula (I) according to any one of claims 1, 2, 3, 4, 5, 6, 7, 8 or 9 and
one or more pharmaceutical active anti cancer compounds or
one or more pharmaceutical active immune checkpoint inhibitors.

14. A pharmaceutical combination according to claim 10, characterized in that the pharmaceutical active immune checkpoint inhibitor is an antibody.

15. Use of a compound of general formula (I) according to any one of claims 1, 2, 3, 4, 5, 6, 7, 8 or 9 for the treatment or prophylaxis of a disease.

16. Use of a compound of general formula (I) according to any one of claims 1, 2, 3, 4, 5, 6, 7, 8 or 9 for the preparation of a medicament for the treatment or prophylaxis of a disease.

17. Use according to claim 15 or 16, wherein the disease is cancer or conditions with dysregulated immune responses or other disorders associated with aberrant MAP4K1 signaling, such as liquid and solid tumours.

18. Use according to claim 15 or 16, wherein the diseases, respectively the disorders are benign hyperplasias, atherosclerotic disorders, sepsis, autoimmune disorders, vascular disorders, viral infections, neurodegenerative disorders, in inflammatory disorders, and male fertility control.
